(19) United States
(12) Patent Application Publication
(10) Pub. No.: US 2025/0179017 A1
(43) Pub. Date: Jun. 5, 2025

(54) CARBOXAMIDE COMPOUNDS AS PGE2 RECEPTOR ANTAGONISTS

(71) Applicant: GUANGDONG NEWOPP BIOPHARMACEUTICALS CO., LTD., Guangzhou (CN)

(72) Inventors: Yongshuai CHAI, Guangzhou (CN); Guorui YAO, La Jolla, CA (US); Nanxin LI, San Diego, CA (US)

(21) Appl. No.: 18/841,312

(22) PCT Filed: Feb. 22, 2023
(86) PCT No.: PCT/CN2023/077578

§ 371 Date: Aug. 23, 2024

Related U.S. Application Data
(60) Provisional application No. 63/475,462, filed on Nov. 10, 2022.
(60) Provisional application No. 63/473,258, filed on May 16, 2022.
(60) Provisional application No. 63/372,204, filed on Feb. 24, 2022.

(51) Int. Cl. C07D 209/08; A61K 31/404; A61K 31/416; C07D 231/56

(52) U.S. Cl. CPC C07D 209/08; A61K 31/404; A61K 31/416; C07D 231/56

(57) ABSTRACT

This disclosure relates to compounds of Formula I as PGE2 receptor antagonists or a pharmaceutically acceptable salt thereof.

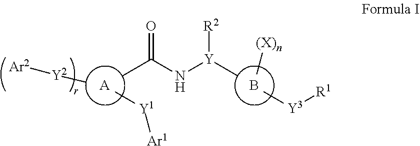

### TECHNICAL FIELD

The present invention is directed to a series of novel carboxamides as PGE2 receptor antagonists useful for the treatment of PGE2 mediated diseases or conditions, such as for pain, inflammation, cancer immunotherapy and neurodegenerative diseases. Pharmaceutical compositions and methods of use are also included.

### BACKGROUND

This invention relates to carboxamides, or their pharmaceutically acceptable salts, pharmaceutical acceptable prodrugs, pharmaceutical compositions containing them, and their medical uses. The compounds of this invention have activity as prostaglandin E₂ (PGE2) receptor antagonists, and are useful in the treatment or alleviation of cancer, pain, inflammation and other inflammation-associated disorders, such as arthritis/rheumatoid arthritis, skin inflammation, vascular inflammation, Alzheimer's disease (AD), Parkinson's diseases (PD), amyotrophic lateral sclerosis (ALS), brain injury, neuropathic pain, hypertension, ischemic or hemorrhagic injury, kidney disease/transplant rejection, atherosclerosis, ischemic heart disease, acne vulgaris, asthma, chronic prostatitis, glomerulonephritis, hypersensitivities, pelvic inflammatory disease, sarcoidosis, vasculitis, interstitial cystitis, preterm delivery, autoimmune diseases, neuroinflammation after a seizure, endometriosis and the like.

Prostaglandins are mediators of pain, fever and other symptoms associated with inflammation. Especially PGE2 is the predominant eicosanoid detected in inflammation conditions. In addition, PGE2 has also been implicated as an important constituent in the immunosuppressive environment created by many solid tumors (Whiteside, Expert Opinion in Biological Therapy, 2010, 1019-1035). PGE2 receptor 2 (EP2) and receptor 4 (EP4) have been shown to be expressed by tumor cells in several cancers, including colon, prostate and breast cancer. Both genetic knockout of EP2/EP4 and blockage of PGE2/EP2 and PGE2/EP4 signaling pathways with EP2 and EP4 antagonists have shown to be a promising therapeutic intervention for several forms of cancer, such as colon cancer, malignant glioma and neuroblastoma. Recent research has revealed that hepatic stellate cells increase the numbers of Th17 cells and regulatory T cells via the PGE2/EP2 pathway (Li at al., J. transl. Med., 2017, 75). Therefore, EP2/EP4 antagonists may improve or synergize with the anti-tumor effect of checkpoint inhibitors, such as anti-CTLA4, anti-PD-L1 or anti-PD-1 antibodies.

This invention relates to a series of novel amide derivatives as PGE2 receptor antagonists, particularly EP2/EP4 antagonists, and methods for treating PGE2 mediated diseases, in particular for cancer, pain, inflammation and neurodegenerative diseases, along with certain pharmaceutical compositions thereof.

### DESCRIPTION

The invention encompasses a genus of compounds of Formula I as PGE2 receptor antagonists or a pharmaceutically acceptable salt thereof,

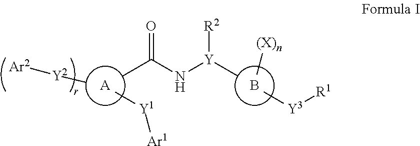

wherein:
R¹ represents —CO₂H, —C(O)NHS(O)₂R⁷, —NHC(O)NHSO₂R⁷, -1H-tetrazolyl, —P(O)(OH)₂, -1,2,4-oxadiazol-5(4H)-one or -tetrazol-5(4H)-one;
Y is N, CR³ or

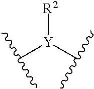

is a bond;
R², R³ are independently selected from the group consisting of: halogen, hydrogen, C₁₋₆alkyl, C₃₋₆cyclolkyl,
C₁₋₆ fluorocycloalkyl, C₁₋₆ fluoroalkyl, or R², R³, together with the carbon atom to which they are both attached to, complete a three-to six-membered carbocyclic ring which is optionally substituted with R^{c} or a three- to six-membered ring which contains one or two heteroatom(s) such as S, O or NR, wherein R is selected from the group consisting of: hydrogen, C₁₋₆alkyl, C₃₋₆cyclolkyl, C₃₋₆fluorocycloalkyl, C₁-₆fluoroalkyl, C₀₋₆alkylene-aryl, C₀₋₆alkylene-heteroaryl, C(O) C₁₋₆alkyl, C(O)aryl, S(O)₂C₁₋₆alkyl, S(O)₂C₃₋₆cycloalkyl, S(O)₂aryl;
X is absent or is independently halogen, —CN, C₁₋₆alkyl, OC₁₋₆alkyl, C₁₋₆haloalkyl C₃₋₆cycloalkyl or OC₁₋₆ haloalkyl;

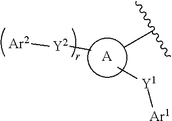

is selected from the following structures:

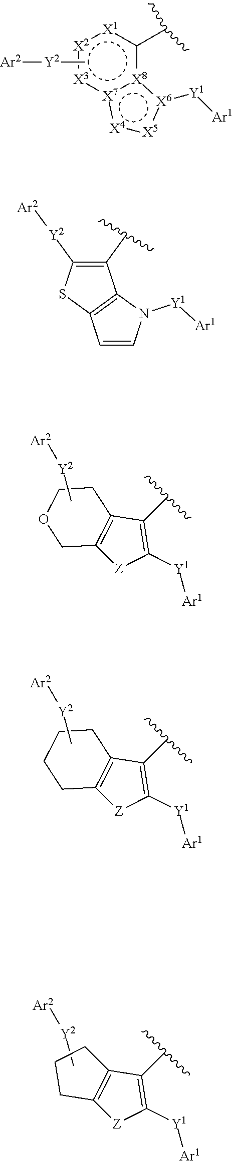

Z is O, S or NR^{B}; X is N or CR⁴;
X², X³ are independently N, CR⁴ or a carbon atom which is connected with —Y²—Ar² with a proviso that at least one of X² or X³ is a carbon atom which is connected with —Y²—Ar²;
X⁴ is O, S, N, NR or CRS;
X⁵ is O, S, N, CR⁶ or —C(O)—;
X⁶, X⁷ and X⁸ are independently N or C;
Y² is —C(O)—, —(CR^{a}R^{1a})ₙ—, —CH═CH, —C≡C—, —O—, —S—, S(O)₂—, —S(O)₂NR^{b}—, —NRS(O)₂—, —C(O)NR—, —NR^{b}C(O)—, NRbC(O)NRW—, NRS(O)₂NR^{b}—, —NR^{b}— or Y² is absent and Ar² is directly connected X² or X³ Y¹ is —O—, —S—, —NR^{b}—, —C(O)—, or —CR^{a}R^{1a}_; Y³ is —(CR^{a}₂)ₘ—;
n and m are independently 0, 1, 2 or 3; r is 1 or 2;
each R and R^{1a} are independently hydrogen, halogen, C₁₋₆alkyl or C₁₋₆haloalkyl, or two R^{a} and R^{1a}, together with the carbon atom to which they are attached to form a 3- to 6-menbered cycloalkyl or heterocyclyl;
R⁴, R⁵, and R⁶ are each independently hydrogen, alkyl, halogen, —OR′, —NR⁸R⁹, —SR⁸, —S(O)₂NR⁸R⁹, —S(O)₂R¹¹, —CN, C₃₋₆cyclolkyl, —C₀₋₆alkylene-aryl, —C₀₋₆alkylene-heteroaryl or haloalkyl;
Ar¹ and Ar² are independently selected from the group consisting of —OR⁸, —SR⁸, NR⁸R⁹, C₁₋₁₀alkyl, C₁₋₁₀haloalkyl, —C₀₋₆alkylene-C₃₋₆cycloalkyl, —C₀₋₆alkylene-aryl, —C₀₋₆alkylene-heteroaryl and —C₀₋₆alkylene-heterocyclyl, or a fused analog of C₃₋₆cycloalkyl; wherein Ar and Ar² are independently substituted with one to three R^{e} groups;
Two Ar² on the adjacent carbon atoms together with carbon atoms can form a three- to six-membered carbocyclic ring, four- to eight-membered bicyclic ring, C₅ to C₁₀ aromatic ring which is optionally substituted with one or more R^{c}, C₂ to C₉₀ heteroaromatic ring which is optionally substituted with one or more R^{c} or a three- to six-membered carbocyclic ring, four- to eight-membered bicyclic ring or five- to eight-membered spirocyclic ring which contains one, two or three heteroatom(s) such as S, O or NR^{b};
each R^{c} is independently halogen, hydrogen, C₁₋₆alkyl, C₃₋₆cycloalkyl, C₁₆fluorocycloalkyl, C₁₋₆fluoroalkyl, —C₂-C₉alkynyl, —C₀₋₆alkylene-heterocyclyl, —C₀₋₆alkylene-aryl, —C₀₋₆alkylene-heteroaryl, SFs, —C₀₋₆alkylene-OR⁶, —C₀₋₆alkylene-P(O)Me₂, —C₀₋₆alkylene-NR⁸R⁹, —C₀₋₆alkylene-C(O)NR⁸R⁹, —C₀₋₆alkylene-NR⁸(O)NR⁸R⁹, —C₀₋₆alkylene-SR^{B}, —C₀₋₆alkylene-S(O)₂NR⁸R⁹, —C₀₋₆alkylene-S(O)₂R¹° or —C₀₋₆alkylene-CN, wherein C₁₋₆ alkyl, C₂₋₉ alkenyl, C₂₋₉ alkynyl, —C₀₋₆alkylene-cycloalkyl, —C₀₋₆alkylene-heterocyclyl, —C₀.₆alkylene-aryl, and —C₀₋₆alkylene-heteroaryl R^{c}optionally substituted with one, two, or three R; R is independently alkyl, haloalkyl, cycloalkyl, cycloalkylalkyl, heterocyclyl, heterocyclylalkyl, halogen, —OR′, —NRR⁹, —CN, —C(O)R¹⁰, —C(O)NRR⁹, —NRBC(O)R¹⁰, —NR⁸C(O)OR⁹, —NR¹⁰ C(O)NR⁸R⁹, —OC(O)NR⁸R⁹, —S(O)₂R¹°, —S(O)R¹⁰, —SR, —S(O)₂NRR⁹, —S(O)NR⁸R⁹, —NRBS(O)R¹⁰, —NRBS(O)₂R¹⁰, or —NR¹⁰ S(O)₂NRR⁹; wherein alkyl is optionally substituted with —OR′ or —NR′R⁹ and wherein cycloalkyl and heterocyclyl are optionally substituted with one, two, or three groups selected from halogen, alkyl, and haloalkyl;
R⁷ is selected from C₁₋₆alkyl, C₁₋₆haloalkyl, C₃₋₆cycloalkyl, C₃₋₆cyclohaloalkyl, aryl and heteroaryl; each R⁸ and each R⁹ are independently hydrogen, alkyl, haloalkyl, cycloalkyl, heterocyclyl, aryl, or heteroaryl, wherein cycloalkyl, heterocyclyl, aryl, and heteroaryl are optionally substituted with one, two, or three groups selected from halogen, alkyl, and haloalkyl; or^{R} and R⁹, together with the atom or atoms to which they are attached, form a heterocyclyl optionally substituted with one, two, or three groups selected from halogen, alkyl, and haloalkyl;
each R¹⁰ is independently alkyl, haloalkyl, cycloalkyl, heterocyclyl, aryl, or heteroaryl, wherein cycloalkyl, heterocyclyl, aryl, and heteroaryl are optionally substituted with one, two, or three groups selected from halogen, alkyl, and haloalkyl;

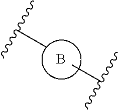

is selected from the following structures:

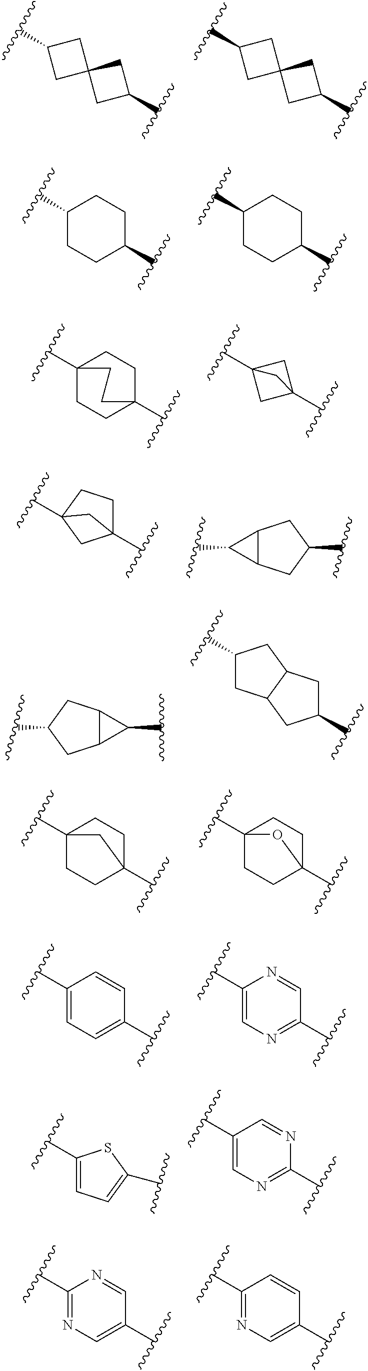

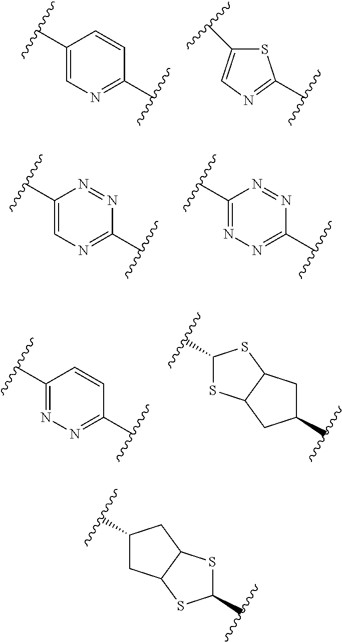

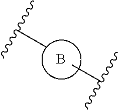

is optionally substituted with one or more W.

In one embodiment,

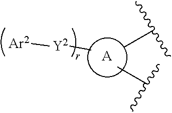

is selected from the following structures:

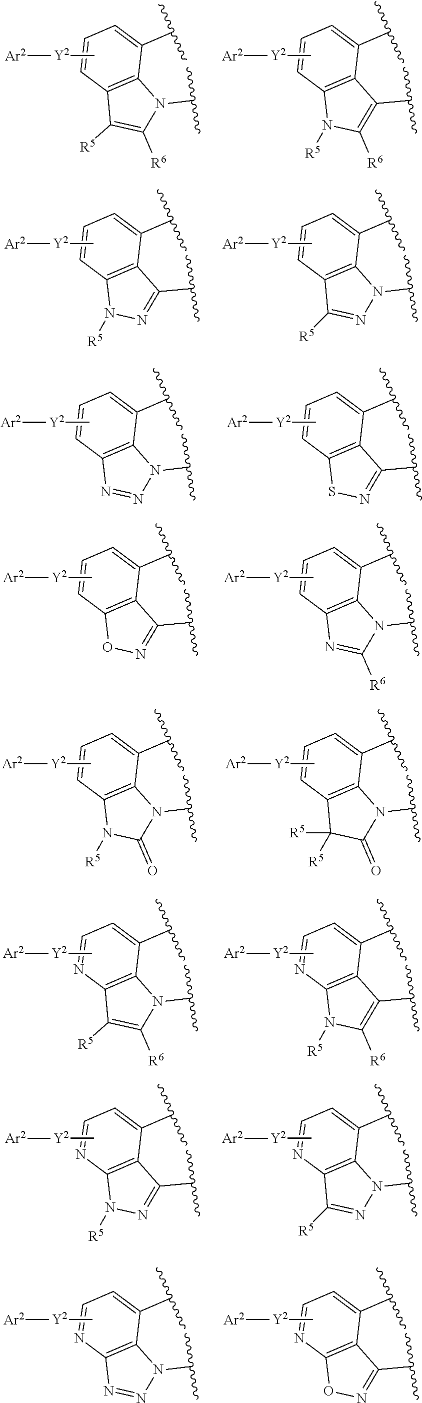

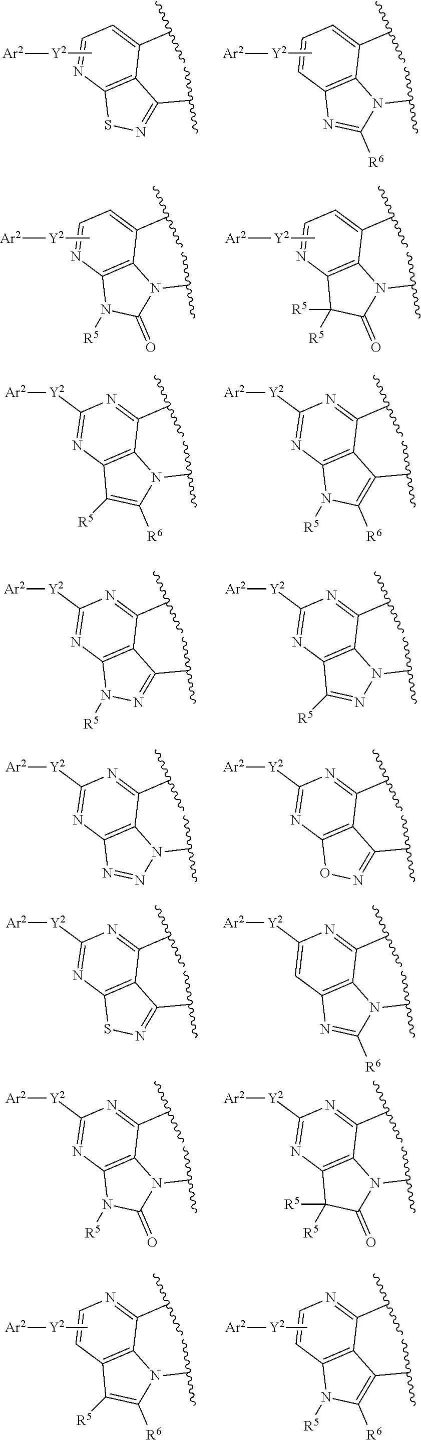

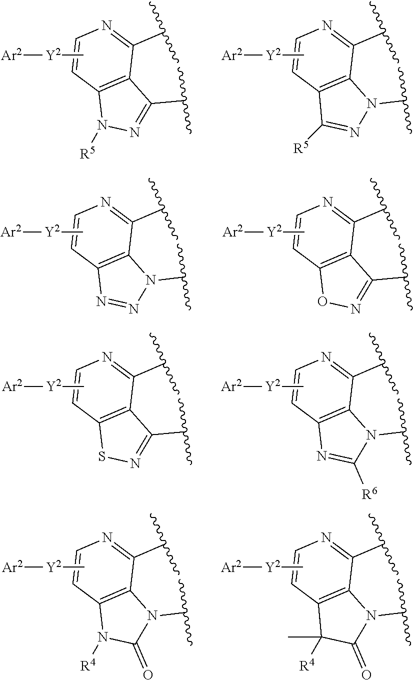

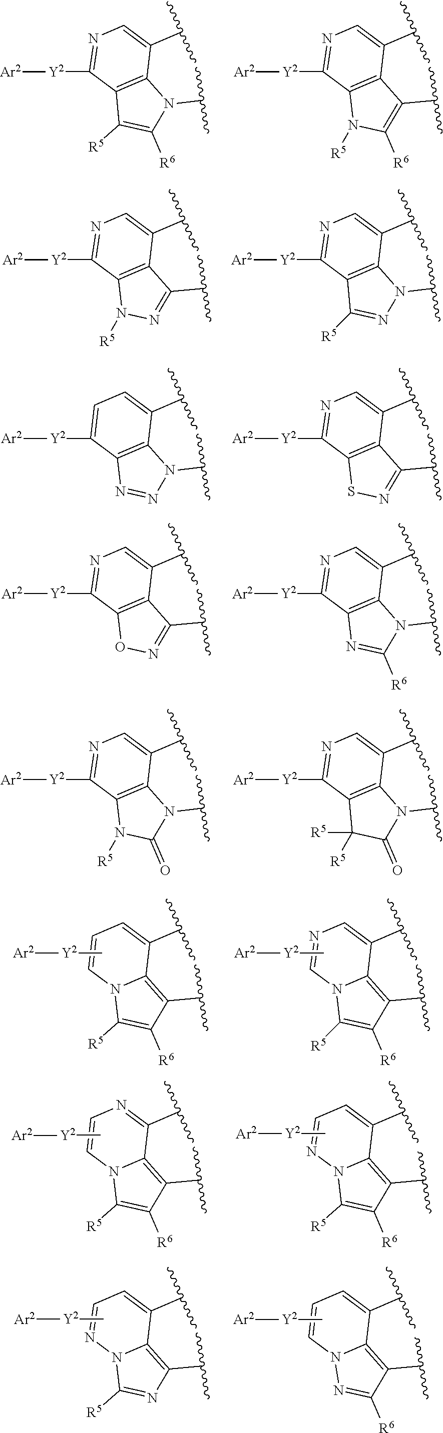

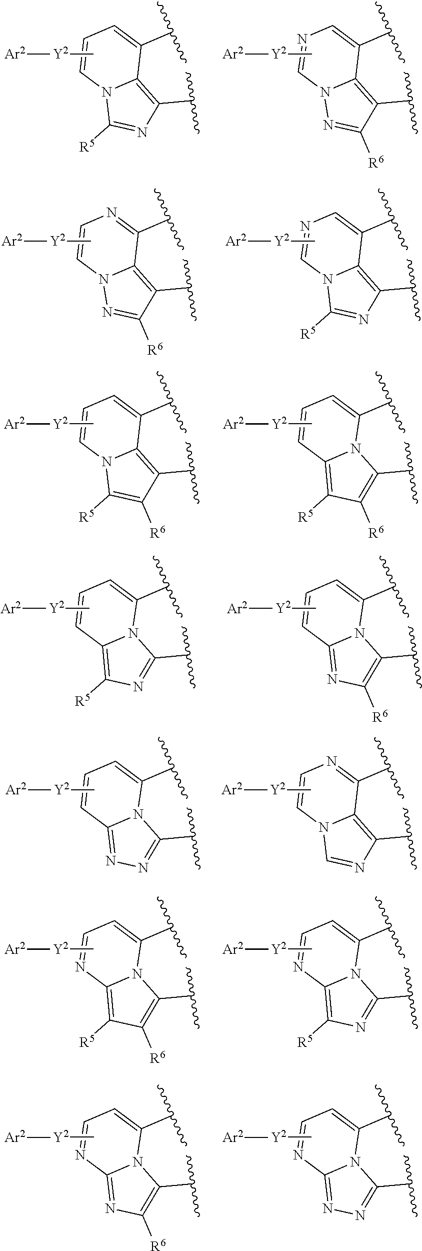

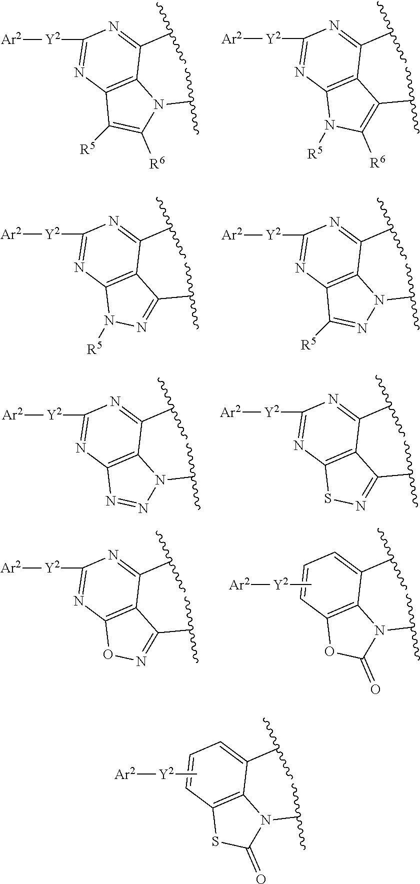

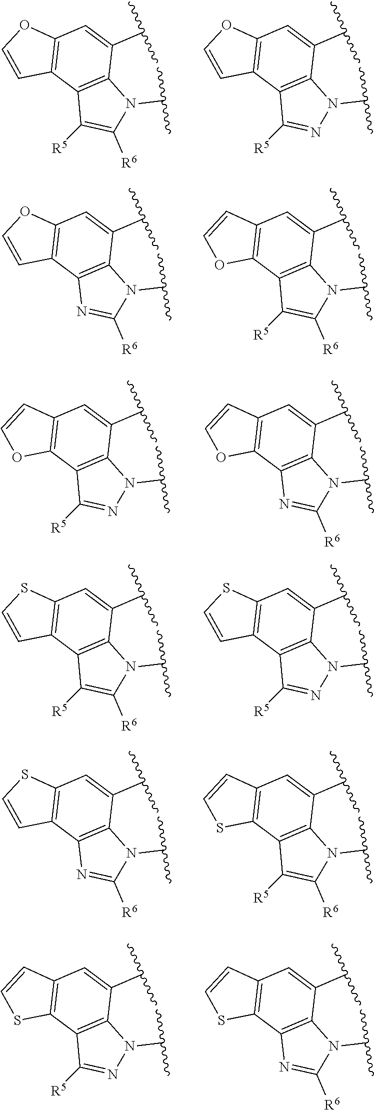

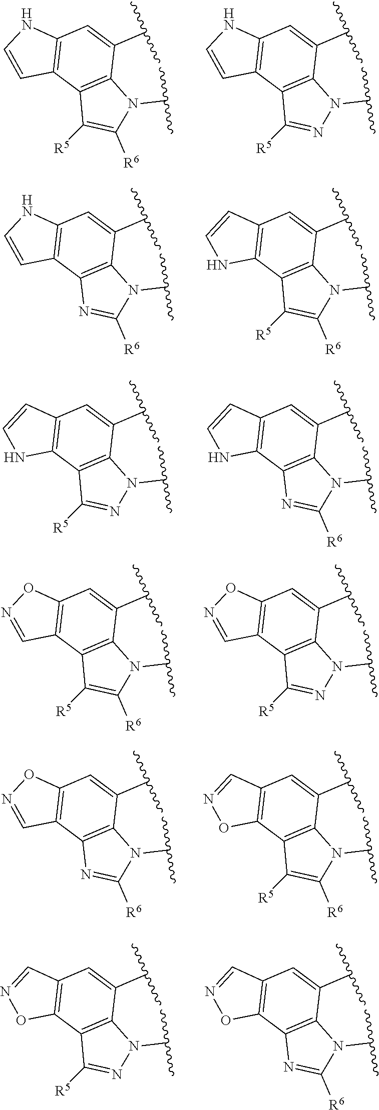

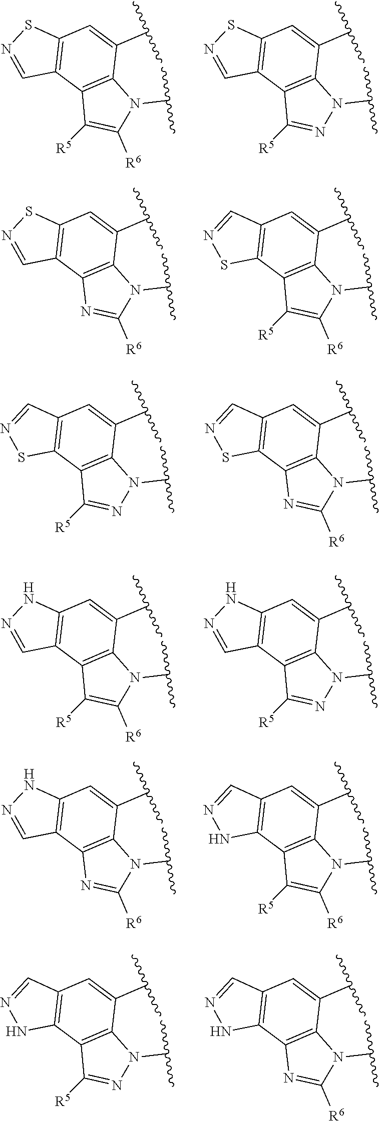

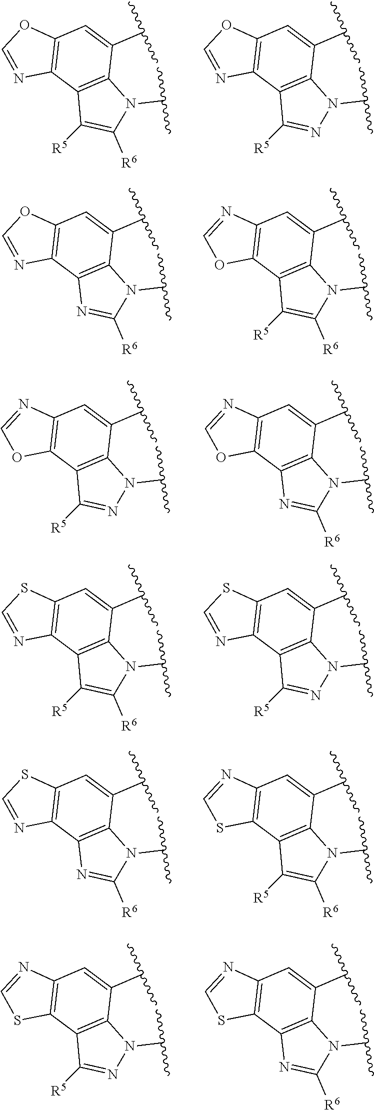

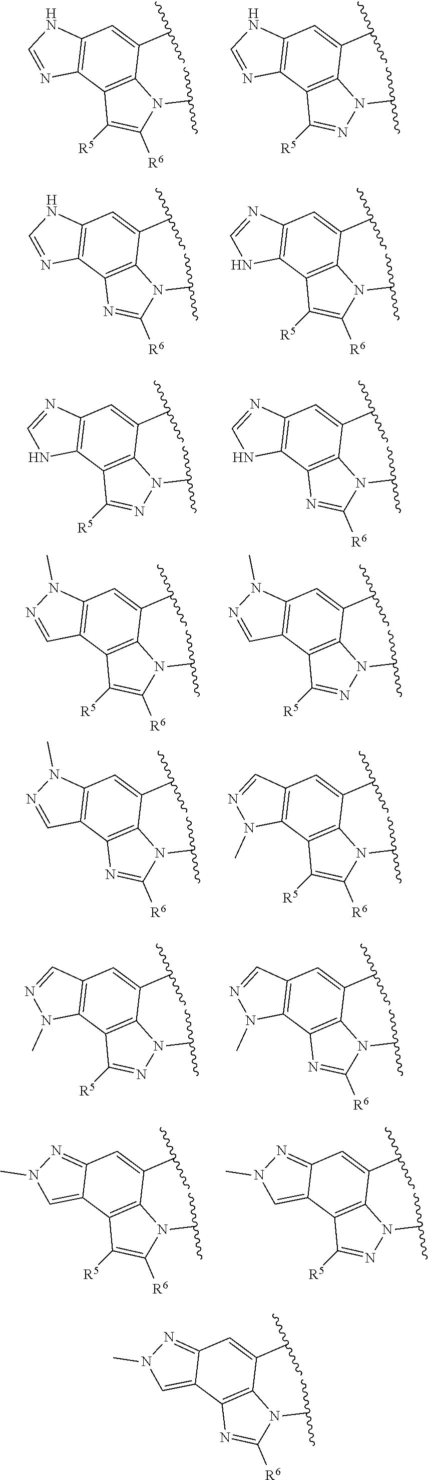

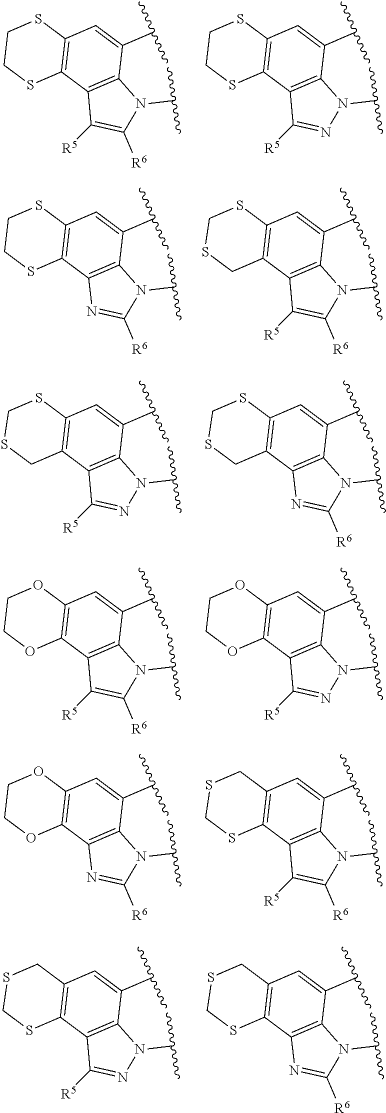

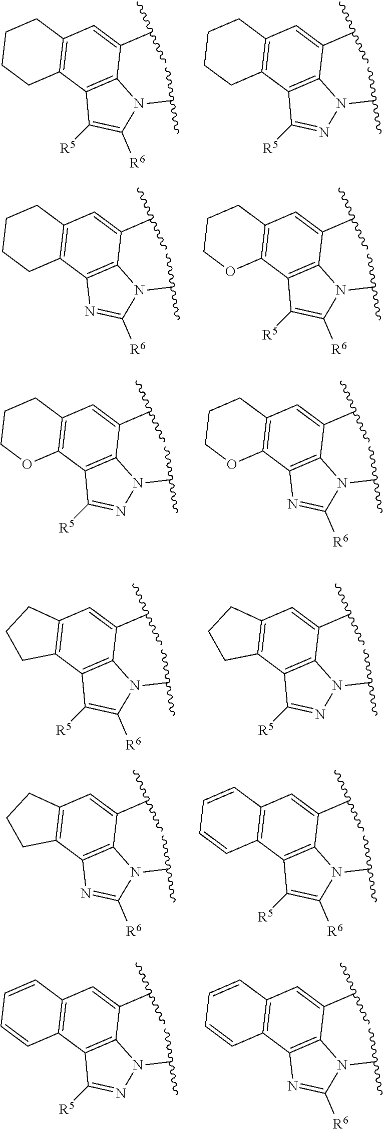

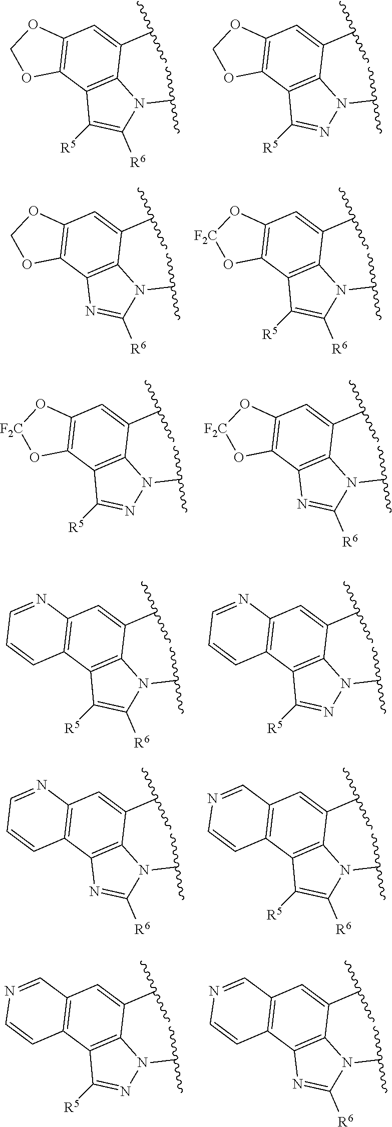

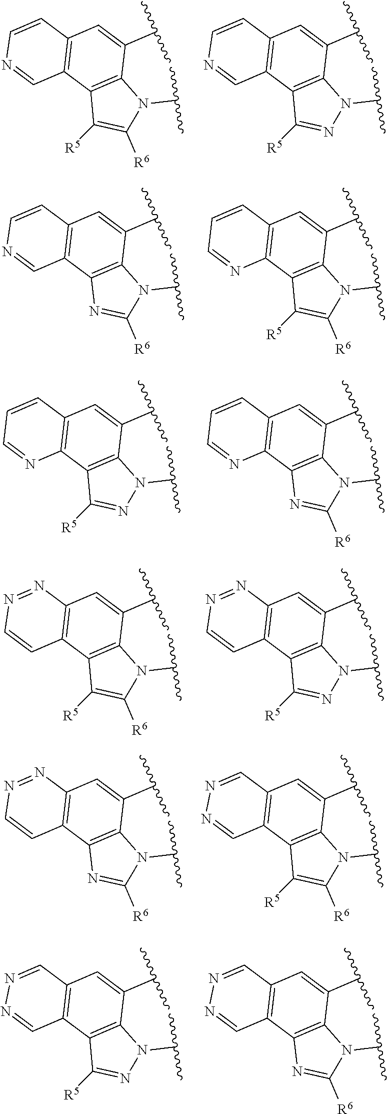

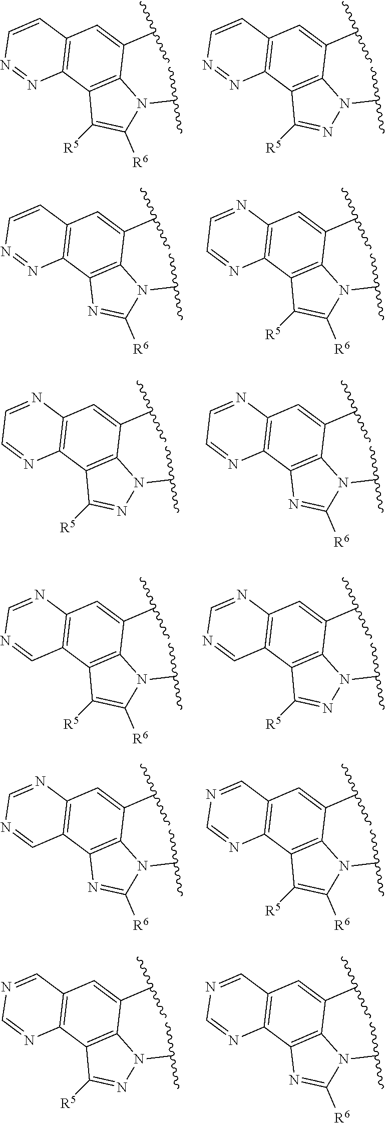

In one embodiment, provided herein are compounds of Formula II or a pharmaceutically acceptable salt thereof:

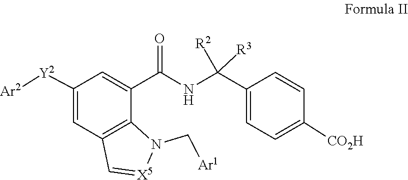

wherein X⁵ is N or CR⁶; R², R³, R⁶, Y², Ar¹ and Ar² are defined as in Formula I.

In an embodiment, the invention further provides a compound of Formula III or a pharmaceutically acceptable salt thereof:

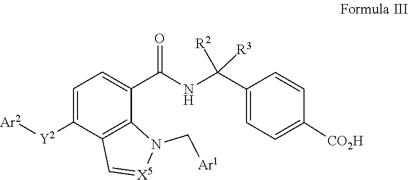

wherein X⁵ is N or CR⁶; R², R³, R⁶, Y², Ar¹ and Ar² are defined as in Formula I.

In an embodiment, the invention further provides a compound of Formula IV or a pharmaceutically acceptable salt thereof:

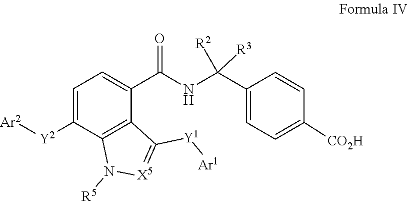

wherein: X⁵ is N or CR⁶; R², R³, R, R⁶, Y¹, Y², Ar¹ and Ar² are defined as in Formula I.

In one embodiment, provided herein are compounds of Formula V or a pharmaceutically acceptable salt thereof:

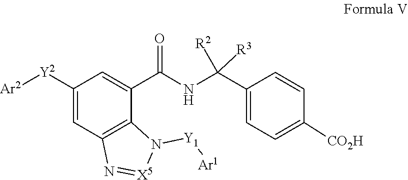

wherein X⁵ is N or CR⁶; R², R³, R⁶, Y¹, Y², Ar¹ and Ar² are defined as in Formula I.

In an embodiment, the invention further provides a compound of Formula VI or a pharmaceutically acceptable salt thereof:

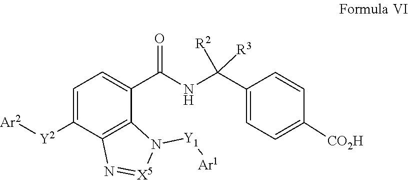

wherein X⁵ is N or CR⁶; R², R³, R⁶, Y¹, Y², Ar¹ and Ar² are defined as in Formula I.

In an embodiment, the invention further provides a compound of Formula VII or a pharmaceutically acceptable salt thereof:

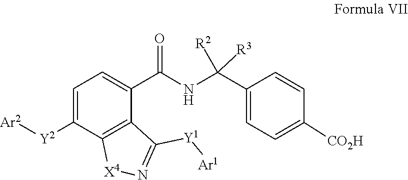

wherein: X⁴ is O or S; R², R³, Y¹, Y², Ar¹ and Ar² are defined as in Formula I.

In one embodiment, provided herein are compounds of Formula VIII or a pharmaceutically acceptable salt thereof:

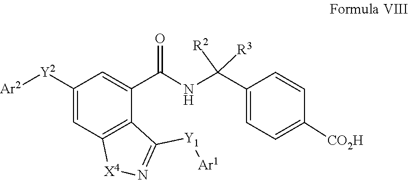

wherein X⁴ is O or S; R², R³, Y¹, Y², Ar¹ and Ar² are defined as in Formula I.

In one embodiment, provided herein are compounds of Formula IX or a pharmaceutically acceptable salt thereof:

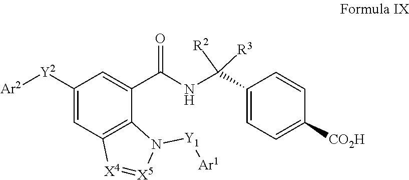

Wherein X⁴, X⁵ is independently N or CR⁶; R², R³, R⁶, Y¹, Y², Ar¹ and Ar² are defined as in Formula I.

In an embodiment, the invention further provides a compound of Formula X or a pharmaceutically acceptable salt thereof:

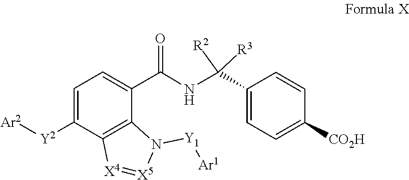

Wherein X⁴, X⁵ is independently N or CR⁶; R², R³, R⁶, Y¹, Y², Ar¹ and Ar² are defined as in Formula I.

In one embodiment, provided herein are compounds of Formula XI or a pharmaceutically acceptable salt thereof:

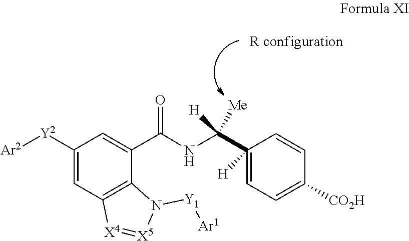

Wherein X⁴, X⁵ is independently N or CR⁶; R⁶, Y¹, Y², Ar¹ and Ar² are defined as in Formula I.

In an embodiment, the invention further provides a compound of Formula XII or a pharmaceutically acceptable salt thereof:

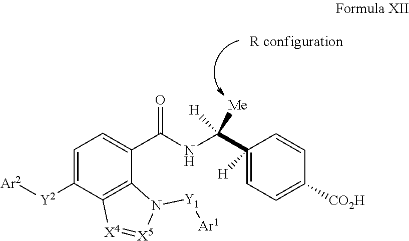

wherein X⁴, X⁵ is independently N or CR⁶; R⁶, Y¹, Y², Ar¹ and Ar² are defined as in Formula I.

In an embodiment, the invention further provides a compound of Formula XIII or a pharmaceutically acceptable salt thereof:

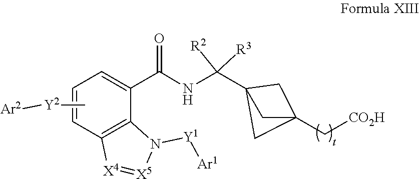

wherein X⁴, X⁵ is independently N or CR⁶; R², R³, R⁶, Y¹, Y², Ar¹ and Ar² are defined as in Formula I. t is 0, 1 or 2.

In an embodiment, the invention further provides a compound of Formula XIV or a pharmaceutically acceptable salt thereof:

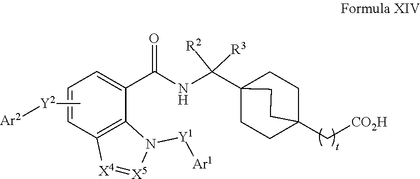

wherein X⁴, X⁵ is independently N or CR⁶; R², R³, R⁶, Y¹, Y², Ar¹ and Ar² are defined as in Formula I. t is 0, 1 or 2.

In an embodiment, the invention further provides a compound of Formula XV or a pharmaceutically acceptable salt thereof:

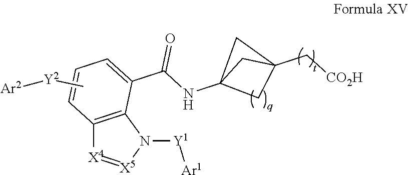

wherein X⁴, X⁵ is independently N or CR⁶; R⁶, Y¹, Y², Ar¹ and Ar² are defined as in Formula I. t and q are independently 0, 1 or 2.

In an embodiment, the invention further provides a compound of Formula XVI or a pharmaceutically acceptable salt thereof

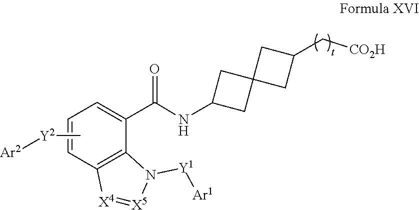

wherein X⁴, X⁵ is independently N or CR⁶; R⁶, Y¹, Y², Ar¹ and Ar² are defined as in Formula I. t is 0, 1 or 2.

In an embodiment, the invention further provides a compound of Formula XVII or a pharmaceutically acceptable salt thereof

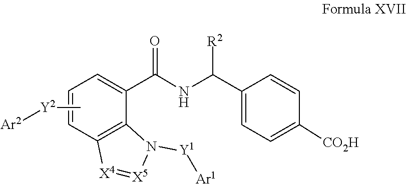

wherein X⁴, X⁵ is independently N or CR; R², R, Y¹, Y², Ar¹ and Ar² are defined as in Formula I.

In some embodiments, a compound of Formula I to XVII is selected from the group consisting of the compounds below or a pharmaceutically acceptable salt thereof:

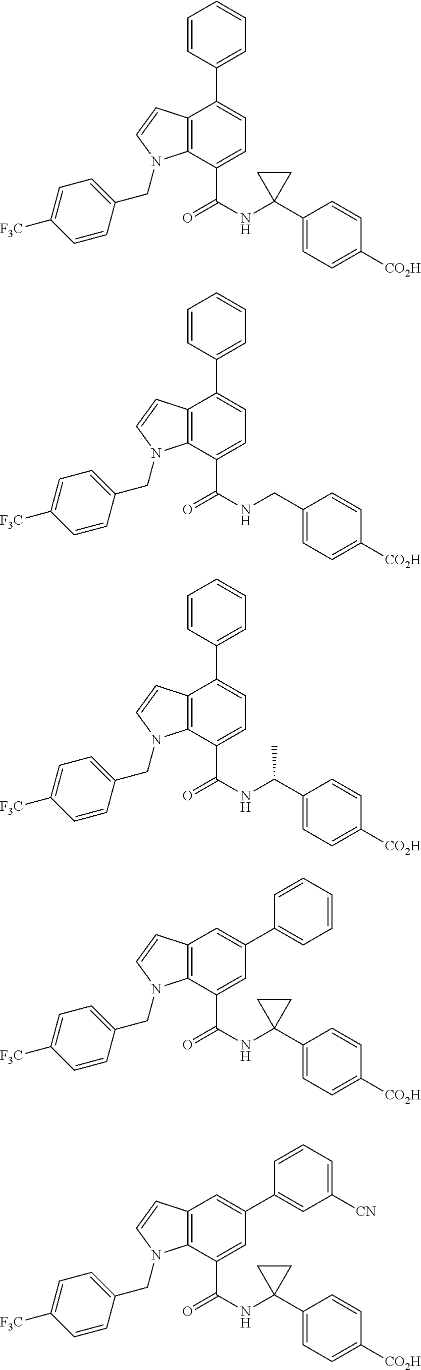

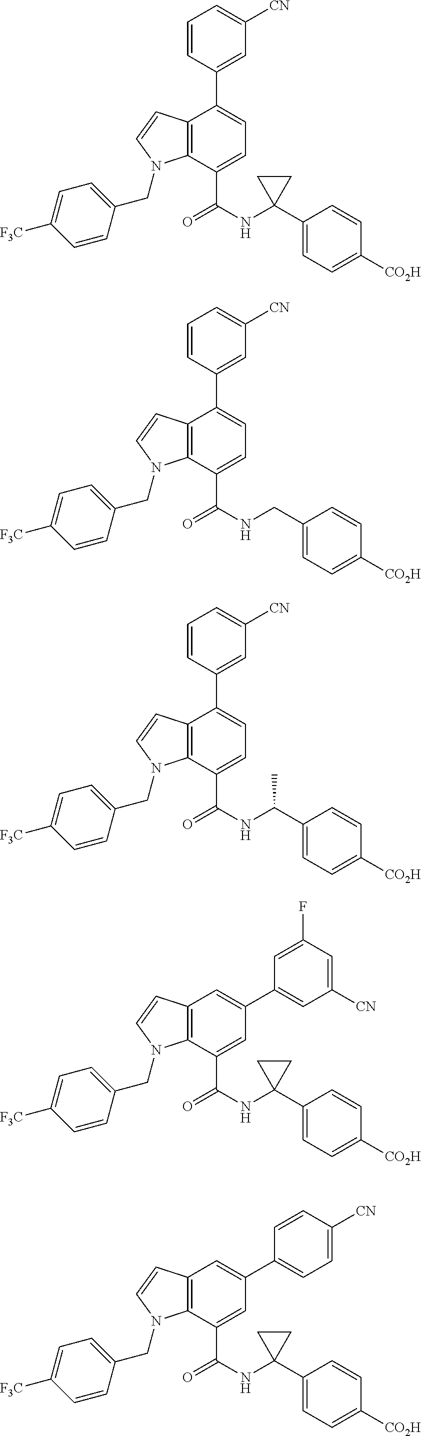

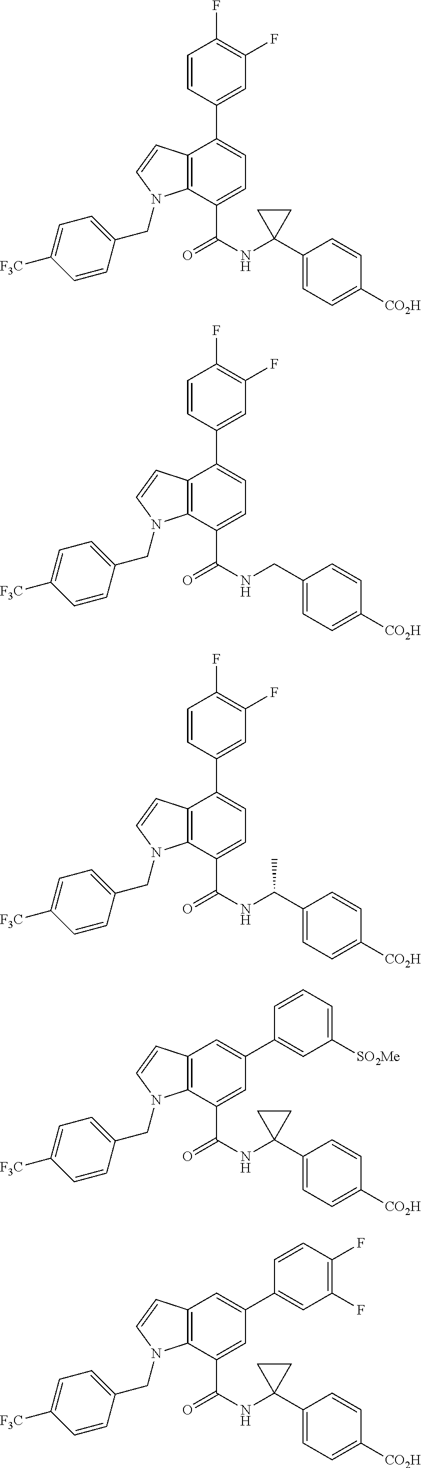

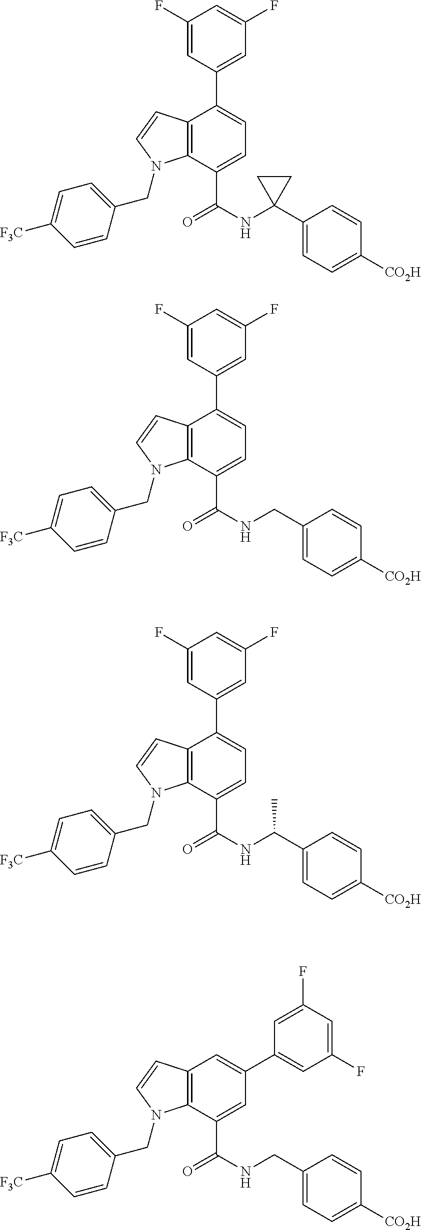 [SMILES: O=C(O)c1ccc(C2(NC(=O)c3ccc(-c4cc(F)cc(F)c4)c4ccn(Cc5ccc(C(F)(F)F)cc5)c34)CC2)cc1 ; O=C(O)c1ccc(CNC(=O)c2ccc(-c3cc(F)cc(F)c3)c3ccn(Cc4ccc(C(F)(F)F)cc4)c23)cc1 ; C[C@@H](NC(=O)c1ccc(-c2cc(F)cc(F)c2)c2ccn(Cc3ccc(C(F)(F)F)cc3)c12)c1ccc(C(=O)O)cc1 ; O=C(O)c1ccc(CNC(=O)c2cc(-c3cc(F)cc(F)c3)cc3ccn(Cc4ccc(C(F)(F)F)cc4)c23)cc1]

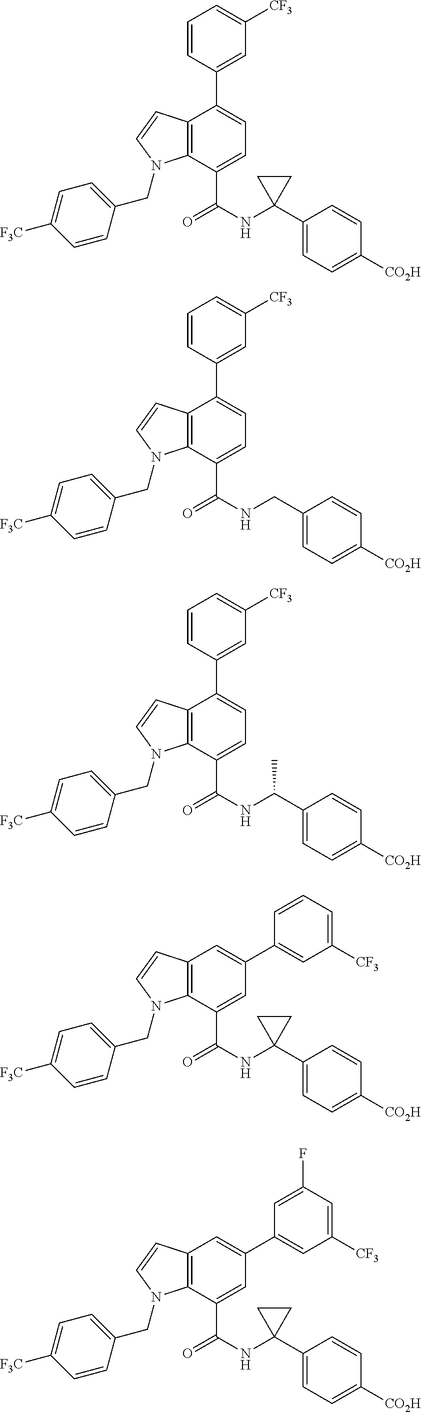

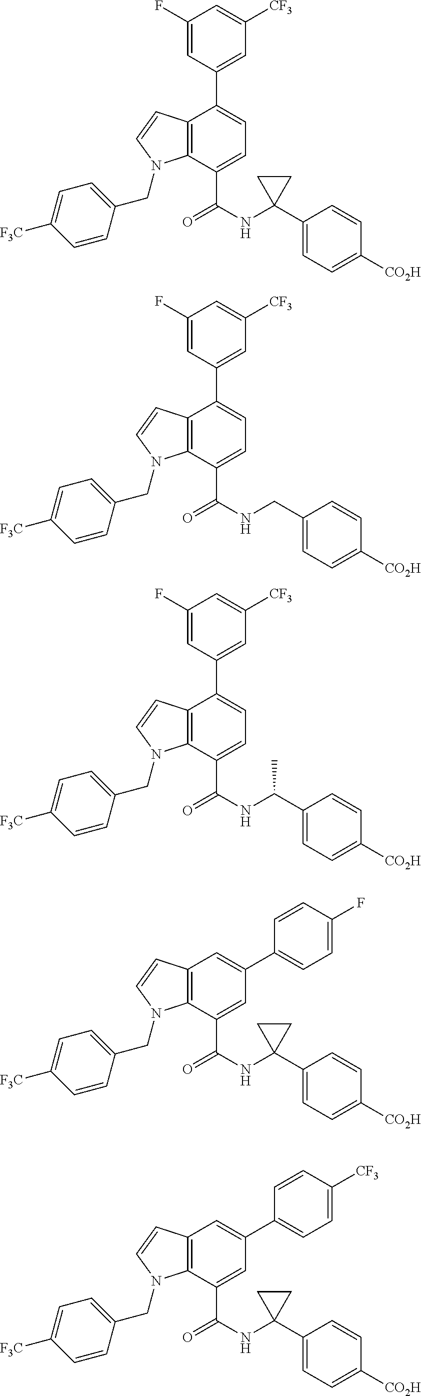 [SMILES: O=C(O)c1ccc(C2(NC(=O)c3ccc(-c4cc(F)cc(C(F)(F)F)c4)c4ccn(Cc5ccc(C(F)(F)F)cc5)c34)CC2)cc1 ; O=C(O)c1ccc(CNC(=O)c2ccc(-c3cc(F)cc(C(F)(F)F)c3)c3ccn(Cc4ccc(C(F)(F)F)cc4)c23)cc1 ; C[C@@H](NC(=O)c1ccc(-c2cc(F)cc(C(F)(F)F)c2)c2ccn(Cc3ccc(C(F)(F)F)cc3)c12)c1ccc(C(=O)O)cc1 ; O=C(O)c1ccc(C2(NC(=O)c3cc(-c4ccc(F)cc4)cc4ccn(Cc5ccc(C(F)(F)F)cc5)c34)CC2)cc1 ; O=C(O)c1ccc(C2(NC(=O)c3cc(-c4ccc(C(F)(F)F)cc4)cc4ccn(Cc5ccc(C(F)(F)F)cc5)c34)CC2)cc1]

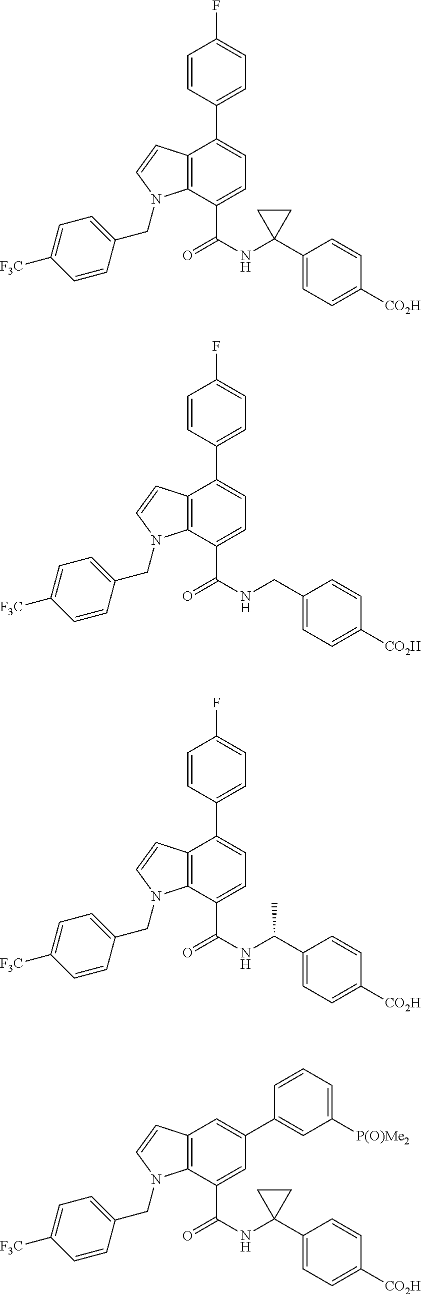

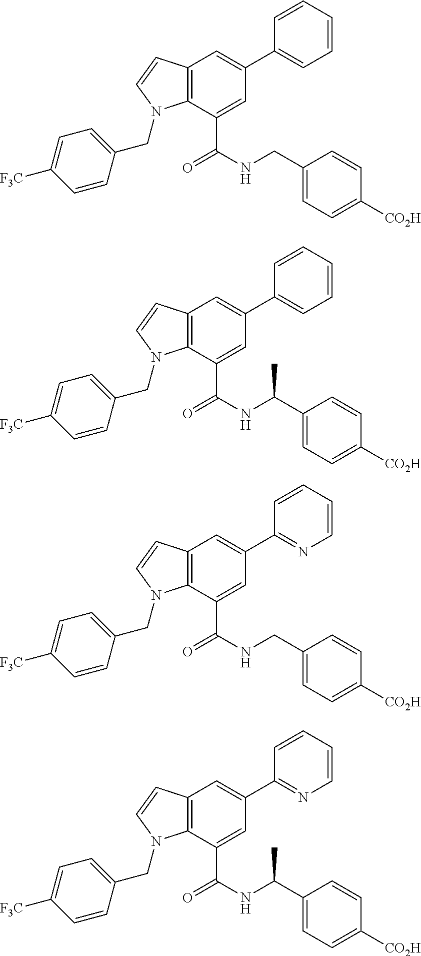

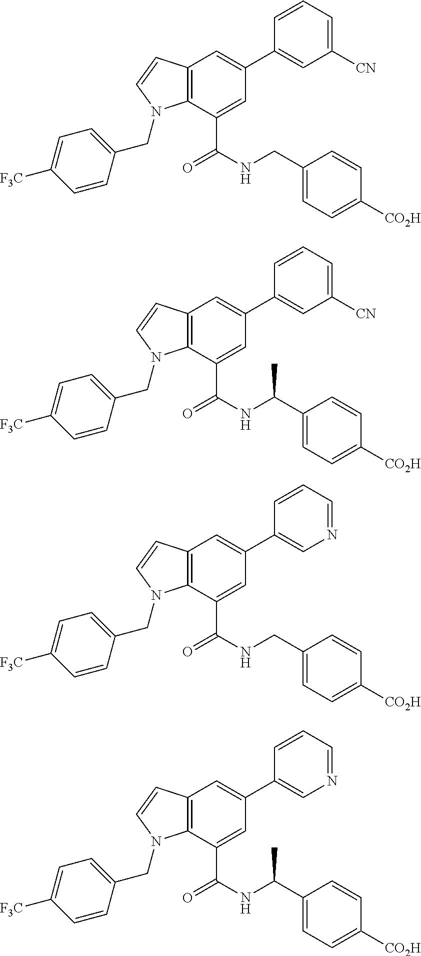

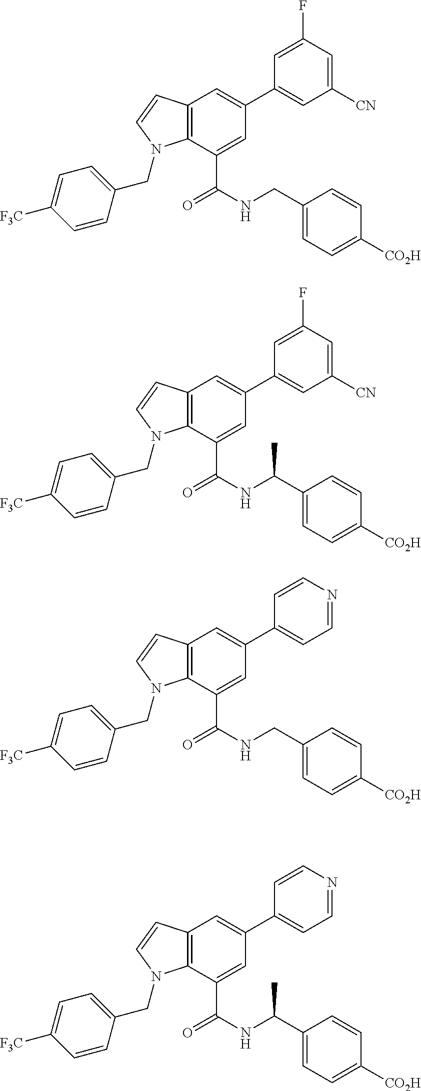

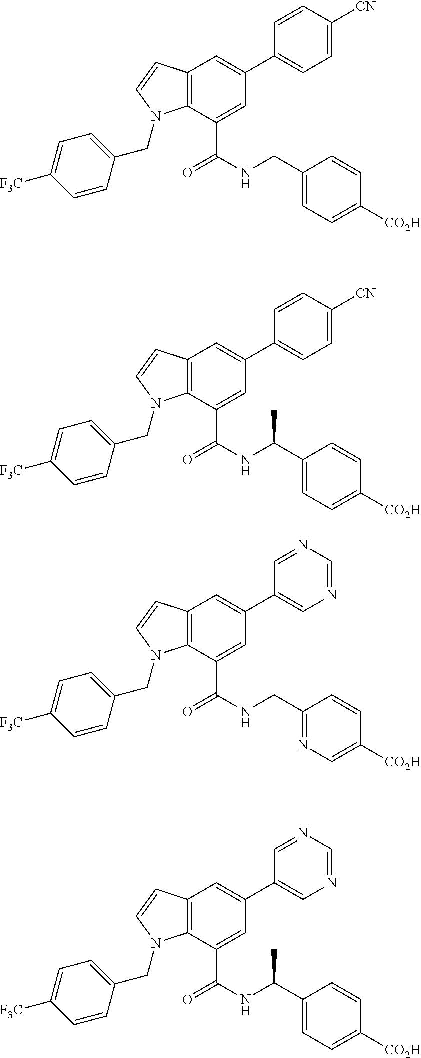

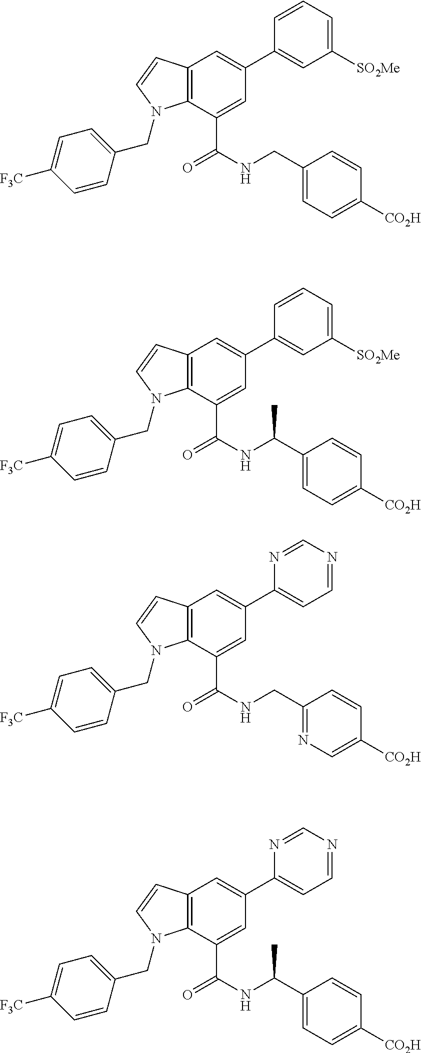

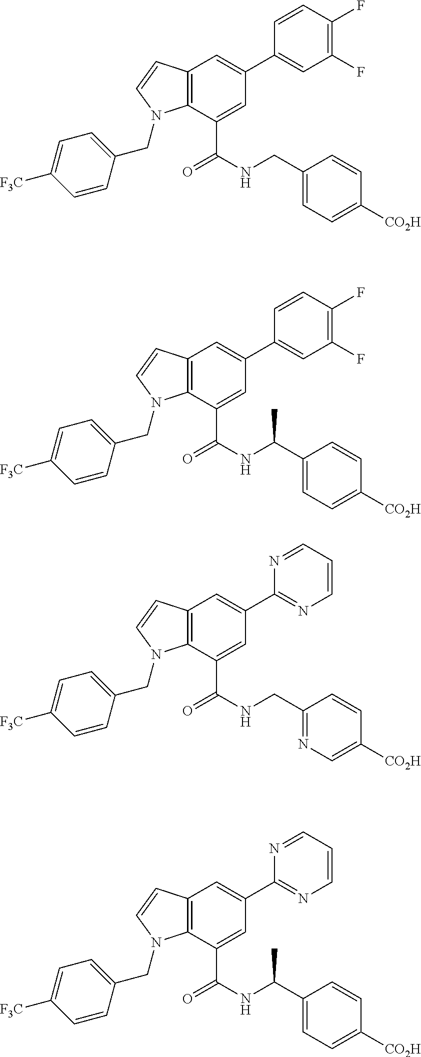

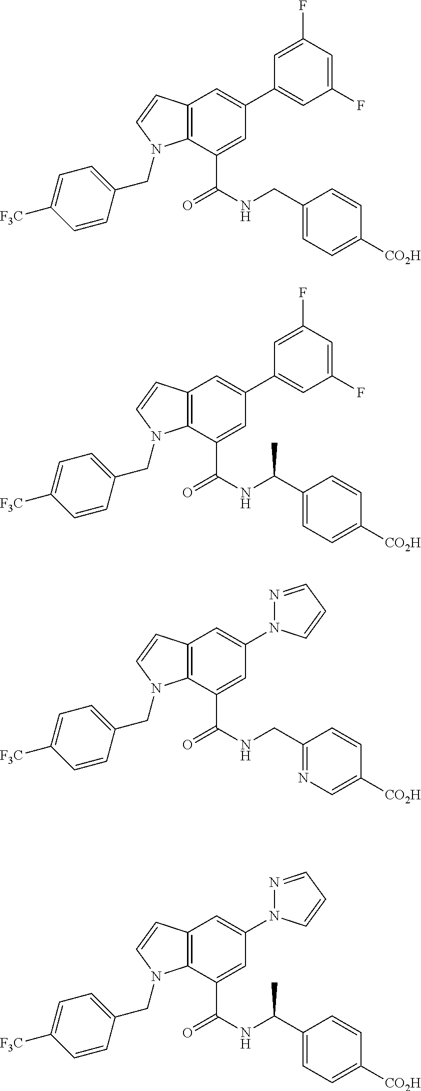 [SMILES: O=C(O)c1ccc(CNC(=O)c2cc(-c3cc(F)cc(F)c3)cc3ccn(Cc4ccc(C(F)(F)F)cc4)c23)cc1 ; C[C@H](NC(=O)c1cc(-c2cc(F)cc(F)c2)cc2ccn(Cc3ccc(C(F)(F)F)cc3)c12)c1ccc(C(=O)O)cc1 ; O=C(O)c1ccc(CNC(=O)c2cc(-n3cccn3)cc3ccn(Cc4ccc(C(F)(F)F)cc4)c23)nc1 ; C[C@H](NC(=O)c1cc(-n2cccn2)cc2ccn(Cc3ccc(C(F)(F)F)cc3)c12)c1ccc(C(=O)O)cc1]

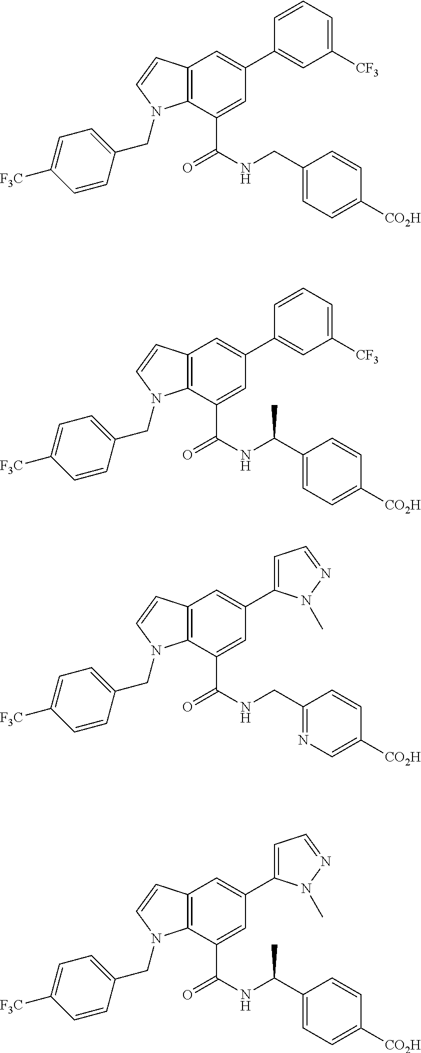 [SMILES: O=C(O)c1ccc(CNC(=O)c2cc(-c3cccc(C(F)(F)F)c3)cc3ccn(Cc4ccc(C(F)(F)F)cc4)c23)cc1 ; C[C@H](NC(=O)c1cc(-c2cccc(C(F)(F)F)c2)cc2ccn(Cc3ccc(C(F)(F)F)cc3)c12)c1ccc(C(=O)O)cc1 ; Cn1nccc1-c1cc(C(=O)NCc2ccc(C(=O)O)cn2)c2c(ccn2Cc2ccc(C(F)(F)F)cc2)c1 ; C[C@H](NC(=O)c1cc(-c2ccnn2C)cc2ccn(Cc3ccc(C(F)(F)F)cc3)c12)c1ccc(C(=O)O)cc1]

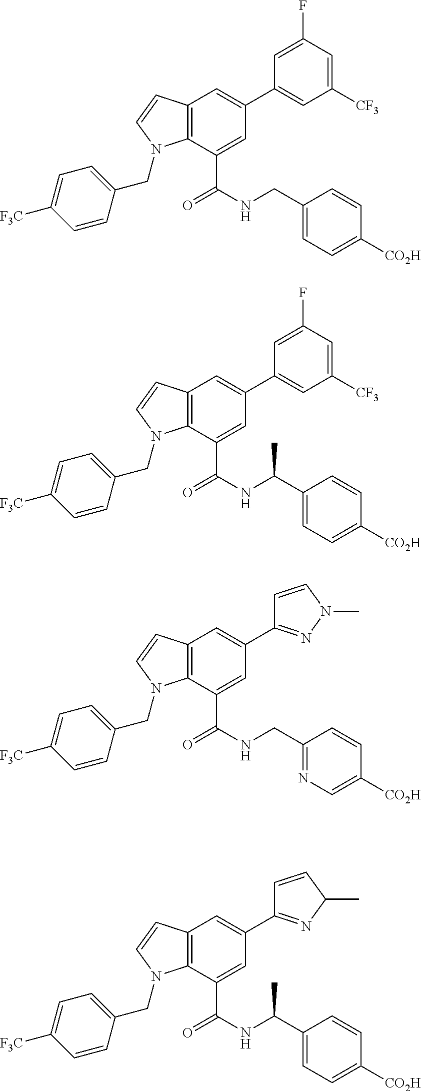 [SMILES: O=C(O)c1ccc(CNC(=O)c2cc(-c3cc(F)cc(C(F)(F)F)c3)cc3ccn(Cc4ccc(C(F)(F)F)cc4)c23)cc1 ; C[C@H](NC(=O)c1cc(-c2cc(F)cc(C(F)(F)F)c2)cc2ccn(Cc3ccc(C(F)(F)F)cc3)c12)c1ccc(C(=O)O)cc1 ; Cn1ccc(-c2cc(C(=O)NCc3ccc(C(=O)O)cn3)c3c(ccn3Cc3ccc(C(F)(F)F)cc3)c2)n1 ; CC1C=CC(c2cc(C(=O)N[C@@H](C)c3ccc(C(=O)O)cc3)c3c(ccn3Cc3ccc(C(F)(F)F)cc3)c2)=N1]

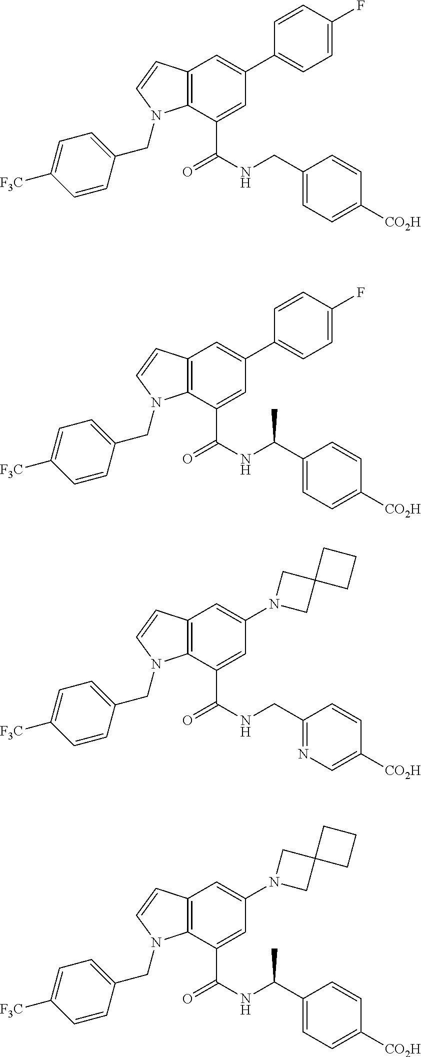

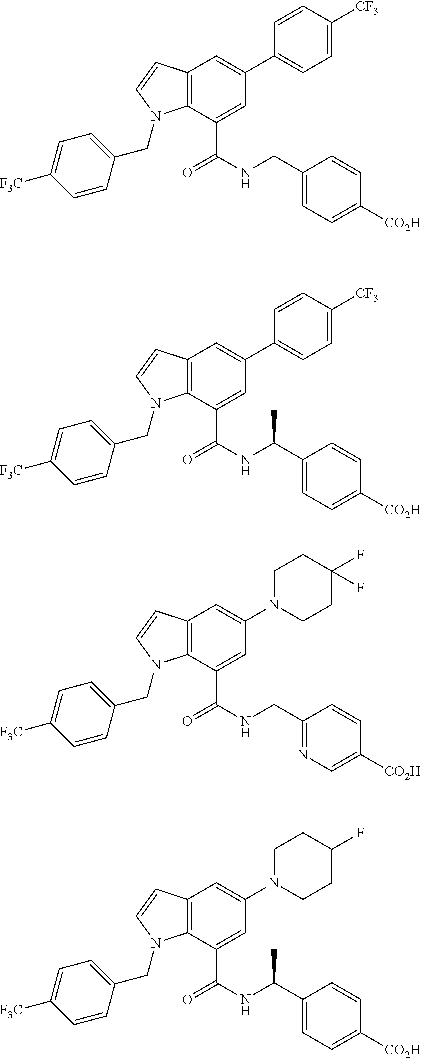 [SMILES: O=C(O)c1ccc(CNC(=O)c2cc(-c3ccc(C(F)(F)F)cc3)cc3ccn(Cc4ccc(C(F)(F)F)cc4)c23)cc1 ; C[C@H](NC(=O)c1cc(-c2ccc(C(F)(F)F)cc2)cc2ccn(Cc3ccc(C(F)(F)F)cc3)c12)c1ccc(C(=O)O)cc1 ; O=C(O)c1ccc(CNC(=O)c2cc(N3CCC(F)(F)CC3)cc3ccn(Cc4ccc(C(F)(F)F)cc4)c23)nc1 ; C[C@H](NC(=O)c1cc(N2CCC(F)CC2)cc2ccn(Cc3ccc(C(F)(F)F)cc3)c12)c1ccc(C(=O)O)cc1]

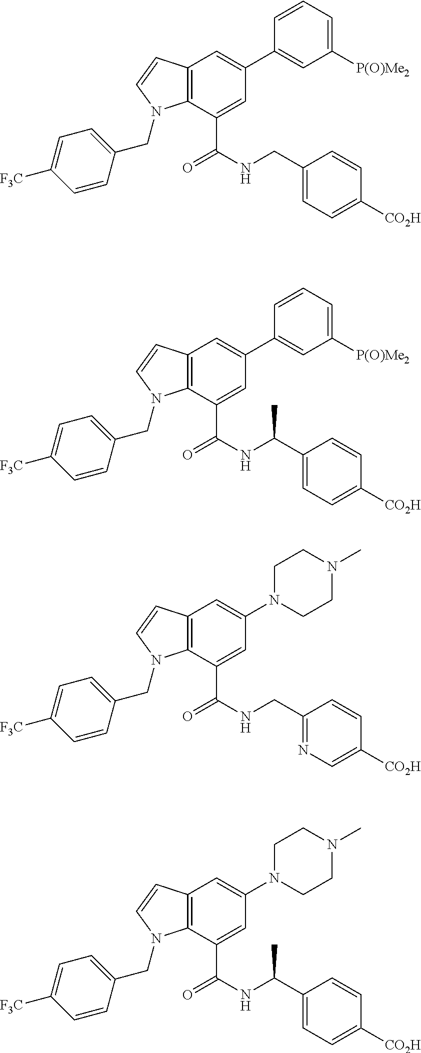

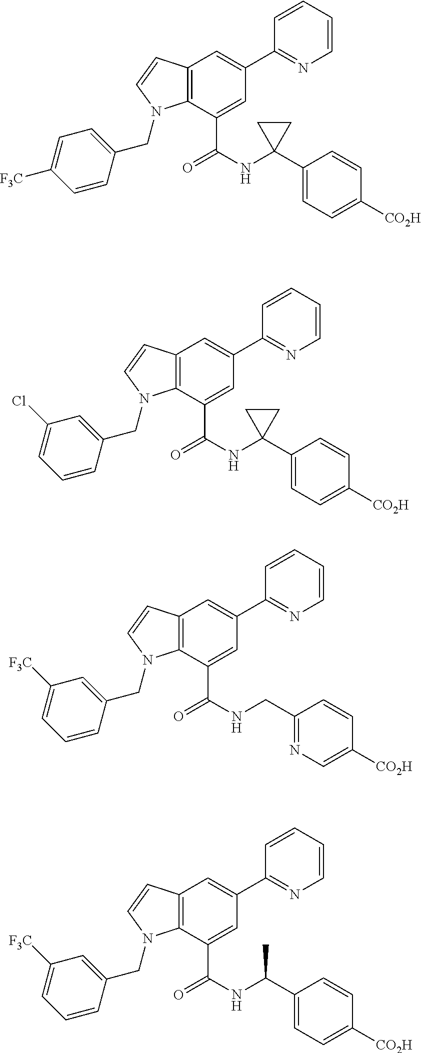 [SMILES: O=C(O)c1ccc(C2(NC(=O)c3cc(-c4ccccn4)cc4ccn(Cc5ccc(C(F)(F)F)cc5)c34)CC2)cc1 ; O=C(O)c1ccc(C2(NC(=O)c3cc(-c4ccccn4)cc4ccn(Cc5cccc(Cl)c5)c34)CC2)cc1 ; O=C(O)c1ccc(CNC(=O)c2cc(-c3ccccn3)cc3ccn(Cc4cccc(C(F)(F)F)c4)c23)nc1 ; C[C@H](NC(=O)c1cc(-c2ccccn2)cc2ccn(Cc3cccc(C(F)(F)F)c3)c12)c1ccc(C(=O)O)cc1]

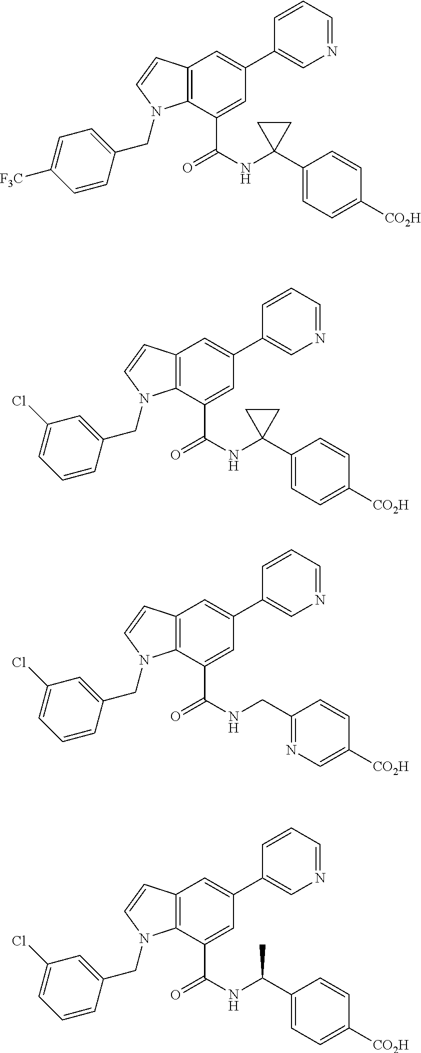 [SMILES: O=C(O)c1ccc(C2(NC(=O)c3cc(-c4cccnc4)cc4ccn(Cc5ccc(C(F)(F)F)cc5)c34)CC2)cc1 ; O=C(O)c1ccc(C2(NC(=O)c3cc(-c4cccnc4)cc4ccn(Cc5cccc(Cl)c5)c34)CC2)cc1 ; O=C(O)c1ccc(CNC(=O)c2cc(-c3cccnc3)cc3ccn(Cc4cccc(Cl)c4)c23)nc1 ; C[C@H](NC(=O)c1cc(-c2cccnc2)cc2ccn(Cc3cccc(Cl)c3)c12)c1ccc(C(=O)O)cc1]

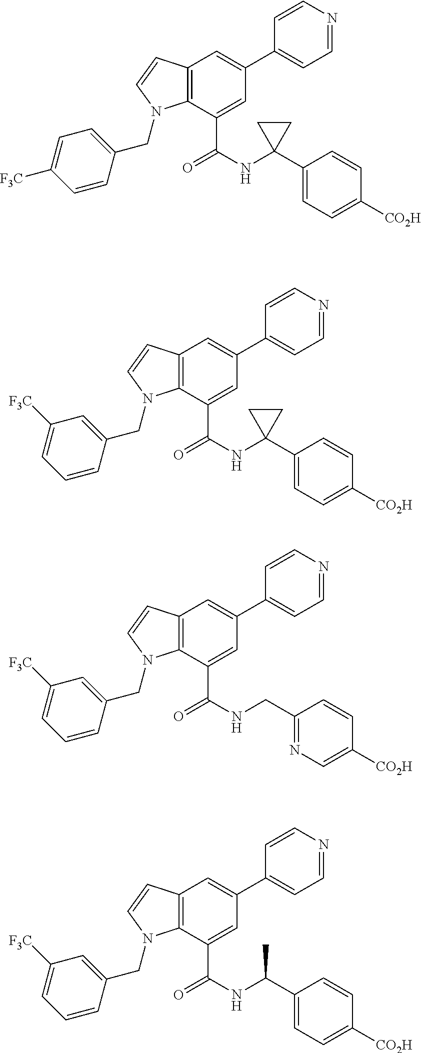 [SMILES: O=C(O)c1ccc(C2(NC(=O)c3cc(-c4ccncc4)cc4ccn(Cc5ccc(C(F)(F)F)cc5)c34)CC2)cc1 ; O=C(O)c1ccc(C2(NC(=O)c3cc(-c4ccncc4)cc4ccn(Cc5cccc(C(F)(F)F)c5)c34)CC2)cc1 ; O=C(O)c1ccc(CNC(=O)c2cc(-c3ccncc3)cc3ccn(Cc4cccc(C(F)(F)F)c4)c23)nc1 ; C[C@H](NC(=O)c1cc(-c2ccncc2)cc2ccn(Cc3cccc(C(F)(F)F)c3)c12)c1ccc(C(=O)O)cc1]

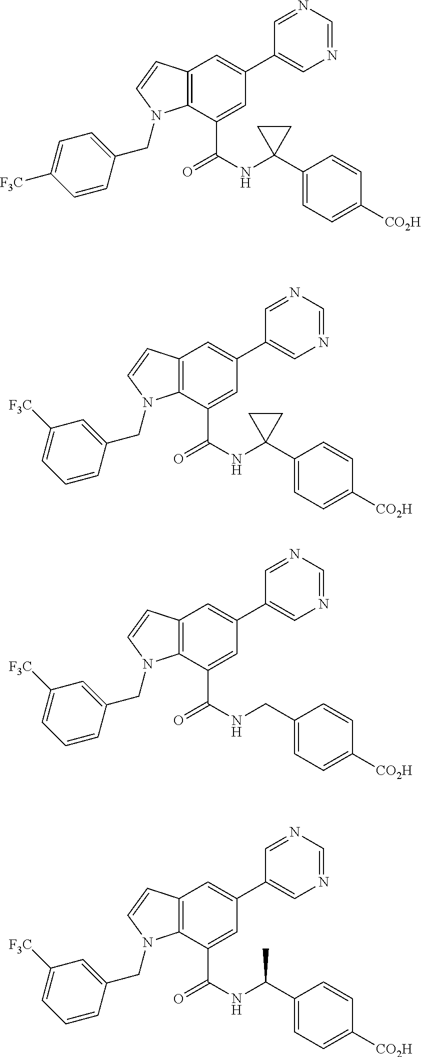 [SMILES: O=C(O)c1ccc(C2(NC(=O)c3cc(-c4cncnc4)cc4ccn(Cc5ccc(C(F)(F)F)cc5)c34)CC2)cc1 ; O=C(O)c1ccc(C2(NC(=O)c3cc(-c4cncnc4)cc4ccn(Cc5cccc(C(F)(F)F)c5)c34)CC2)cc1 ; O=C(O)c1ccc(CNC(=O)c2cc(-c3cncnc3)cc3ccn(Cc4cccc(C(F)(F)F)c4)c23)cc1 ; C[C@H](NC(=O)c1cc(-c2cncnc2)cc2ccn(Cc3cccc(C(F)(F)F)c3)c12)c1ccc(C(=O)O)cc1]

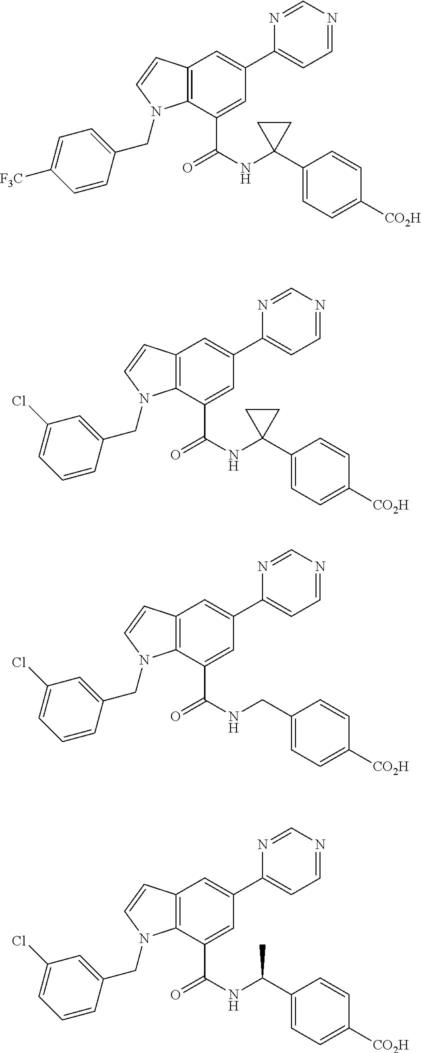 [SMILES: O=C(O)c1ccc(C2(NC(=O)c3cc(-c4ccncn4)cc4ccn(Cc5ccc(C(F)(F)F)cc5)c34)CC2)cc1 ; O=C(O)c1ccc(C2(NC(=O)c3cc(-c4ccncn4)cc4ccn(Cc5cccc(Cl)c5)c34)CC2)cc1 ; O=C(O)c1ccc(CNC(=O)c2cc(-c3ccncn3)cc3ccn(Cc4cccc(Cl)c4)c23)cc1 ; C[C@H](NC(=O)c1cc(-c2ccncn2)cc2ccn(Cc3cccc(Cl)c3)c12)c1ccc(C(=O)O)cc1]

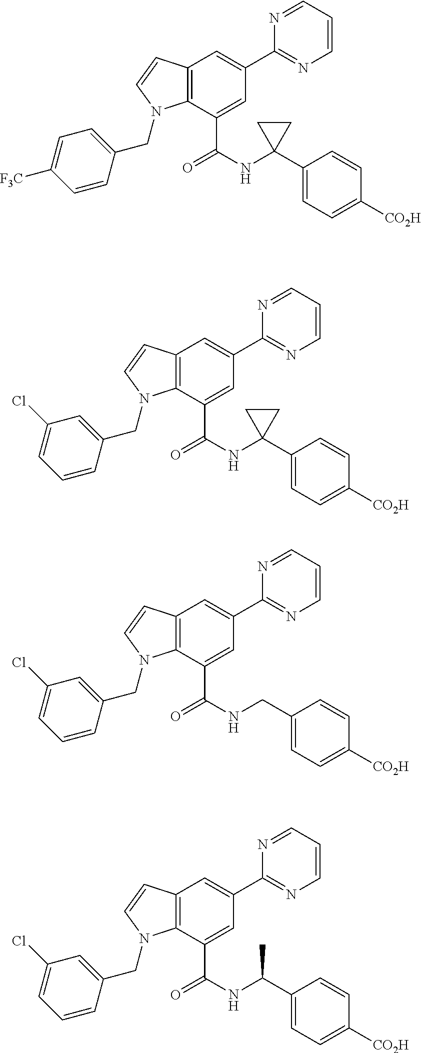 [SMILES: O=C(O)c1ccc(C2(NC(=O)c3cc(-c4ncccn4)cc4ccn(Cc5ccc(C(F)(F)F)cc5)c34)CC2)cc1 ; O=C(O)c1ccc(C2(NC(=O)c3cc(-c4ncccn4)cc4ccn(Cc5cccc(Cl)c5)c34)CC2)cc1 ; O=C(O)c1ccc(CNC(=O)c2cc(-c3ncccn3)cc3ccn(Cc4cccc(Cl)c4)c23)cc1 ; C[C@H](NC(=O)c1cc(-c2ncccn2)cc2ccn(Cc3cccc(Cl)c3)c12)c1ccc(C(=O)O)cc1]

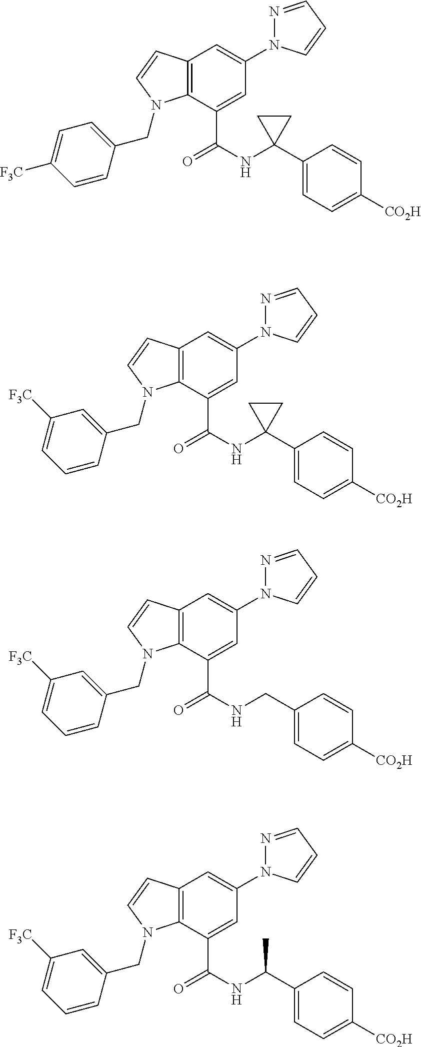 [SMILES: O=C(O)c1ccc(C2(NC(=O)c3cc(-n4cccn4)cc4ccn(Cc5ccc(C(F)(F)F)cc5)c34)CC2)cc1 ; O=C(O)c1ccc(C2(NC(=O)c3cc(-n4cccn4)cc4ccn(Cc5cccc(C(F)(F)F)c5)c34)CC2)cc1 ; O=C(O)c1ccc(CNC(=O)c2cc(-n3cccn3)cc3ccn(Cc4cccc(C(F)(F)F)c4)c23)cc1 ; C[C@H](NC(=O)c1cc(-n2cccn2)cc2ccn(Cc3cccc(C(F)(F)F)c3)c12)c1ccc(C(=O)O)cc1]

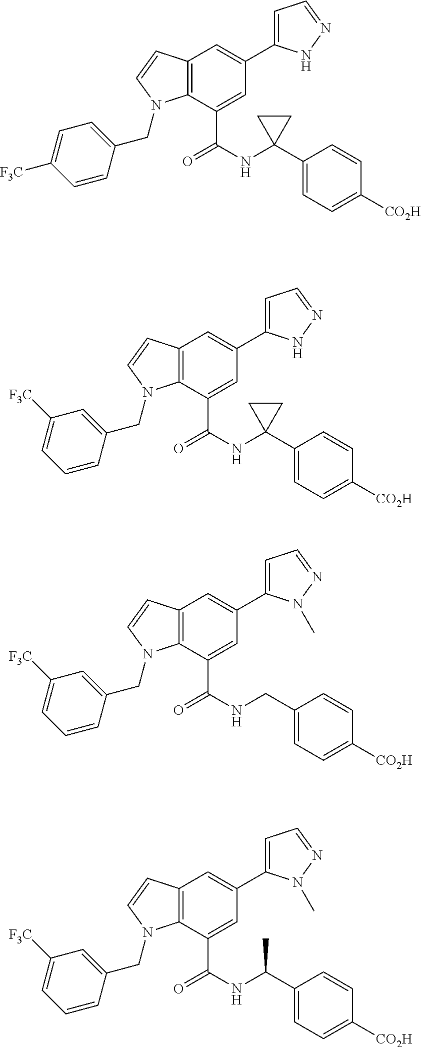 [SMILES: O=C(O)c1ccc(C2(NC(=O)c3cc(-c4ccn[nH]4)cc4ccn(Cc5ccc(C(F)(F)F)cc5)c34)CC2)cc1 ; O=C(O)c1ccc(C2(NC(=O)c3cc(-c4ccn[nH]4)cc4ccn(Cc5cccc(C(F)(F)F)c5)c34)CC2)cc1 ; Cn1nccc1-c1cc(C(=O)NCc2ccc(C(=O)O)cc2)c2c(ccn2Cc2cccc(C(F)(F)F)c2)c1 ; C[C@H](NC(=O)c1cc(-c2ccnn2C)cc2ccn(Cc3cccc(C(F)(F)F)c3)c12)c1ccc(C(=O)O)cc1]

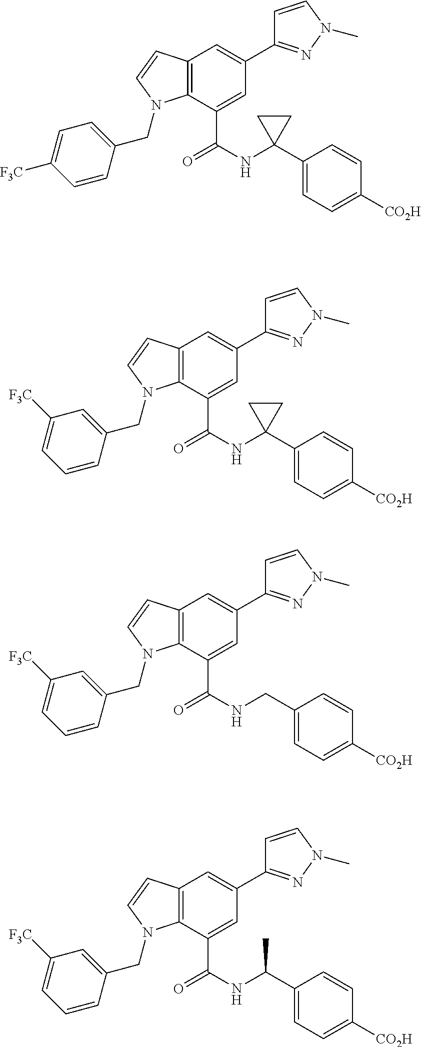 [SMILES: Cn1ccc(-c2cc(C(=O)NC3(c4ccc(C(=O)O)cc4)CC3)c3c(ccn3Cc3ccc(C(F)(F)F)cc3)c2)n1 ; Cn1ccc(-c2cc(C(=O)NC3(c4ccc(C(=O)O)cc4)CC3)c3c(ccn3Cc3cccc(C(F)(F)F)c3)c2)n1 ; Cn1ccc(-c2cc(C(=O)NCc3ccc(C(=O)O)cc3)c3c(ccn3Cc3cccc(C(F)(F)F)c3)c2)n1 ; C[C@H](NC(=O)c1cc(-c2ccn(C)n2)cc2ccn(Cc3cccc(C(F)(F)F)c3)c12)c1ccc(C(=O)O)cc1]

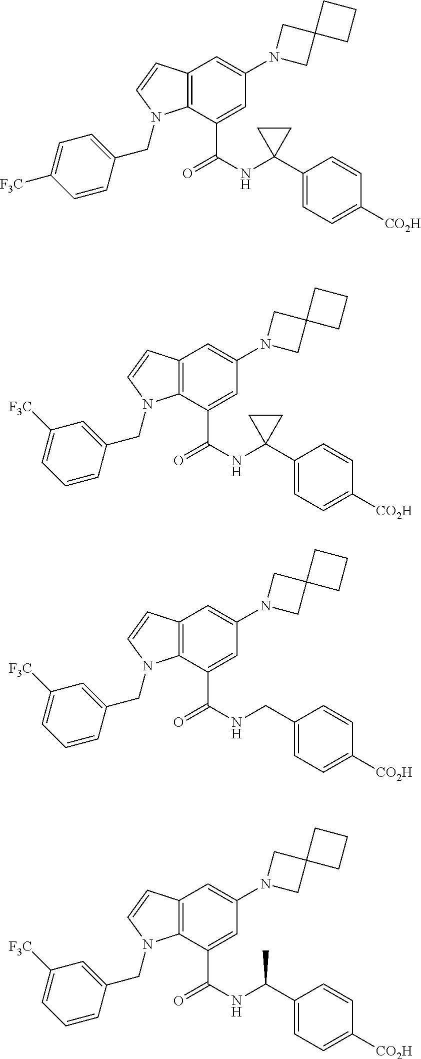

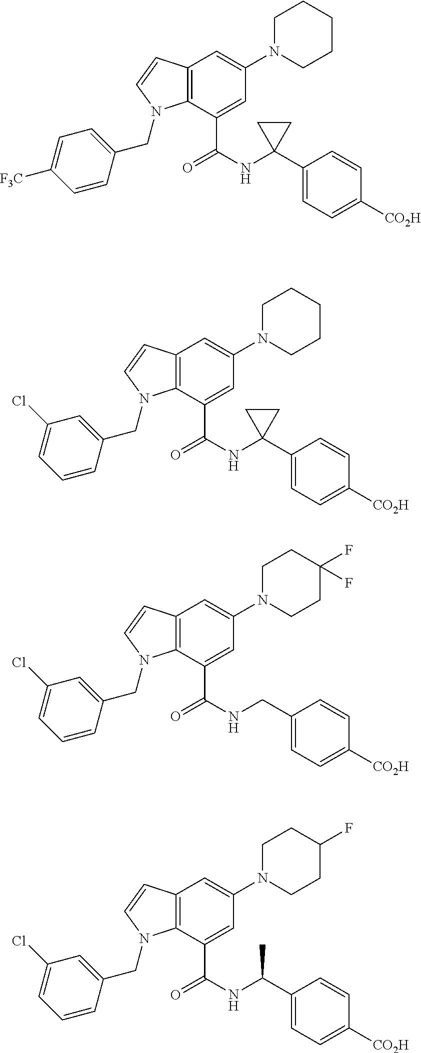 [SMILES: O=C(O)c1ccc(C2(NC(=O)c3cc(N4CCCCC4)cc4ccn(Cc5ccc(C(F)(F)F)cc5)c34)CC2)cc1 ; O=C(O)c1ccc(C2(NC(=O)c3cc(N4CCCCC4)cc4ccn(Cc5cccc(Cl)c5)c34)CC2)cc1 ; O=C(O)c1ccc(CNC(=O)c2cc(N3CCC(F)(F)CC3)cc3ccn(Cc4cccc(Cl)c4)c23)cc1 ; C[C@H](NC(=O)c1cc(N2CCC(F)CC2)cc2ccn(Cc3cccc(Cl)c3)c12)c1ccc(C(=O)O)cc1]

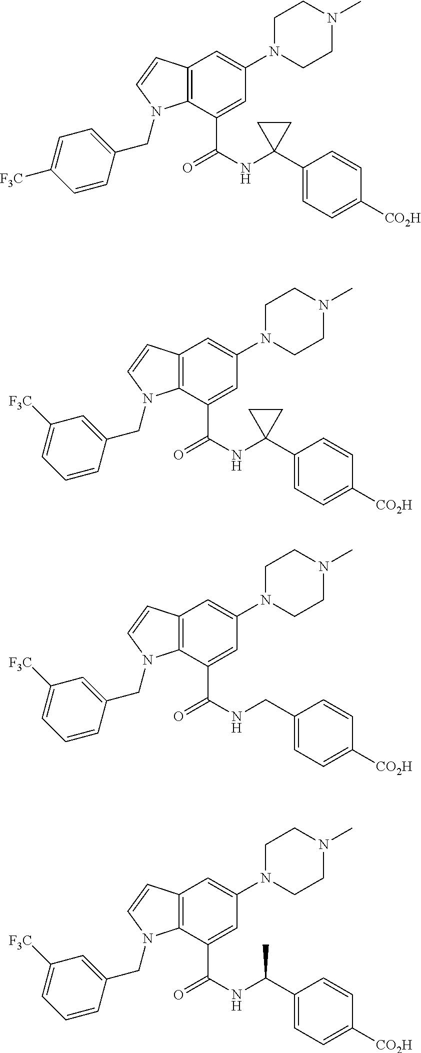 [SMILES: CN1CCN(c2cc(C(=O)NC3(c4ccc(C(=O)O)cc4)CC3)c3c(ccn3Cc3ccc(C(F)(F)F)cc3)c2)CC1 ; CN1CCN(c2cc(C(=O)NC3(c4ccc(C(=O)O)cc4)CC3)c3c(ccn3Cc3cccc(C(F)(F)F)c3)c2)CC1 ; CN1CCN(c2cc(C(=O)NCc3ccc(C(=O)O)cc3)c3c(ccn3Cc3cccc(C(F)(F)F)c3)c2)CC1 ; C[C@H](NC(=O)c1cc(N2CCN(C)CC2)cc2ccn(Cc3cccc(C(F)(F)F)c3)c12)c1ccc(C(=O)O)cc1]

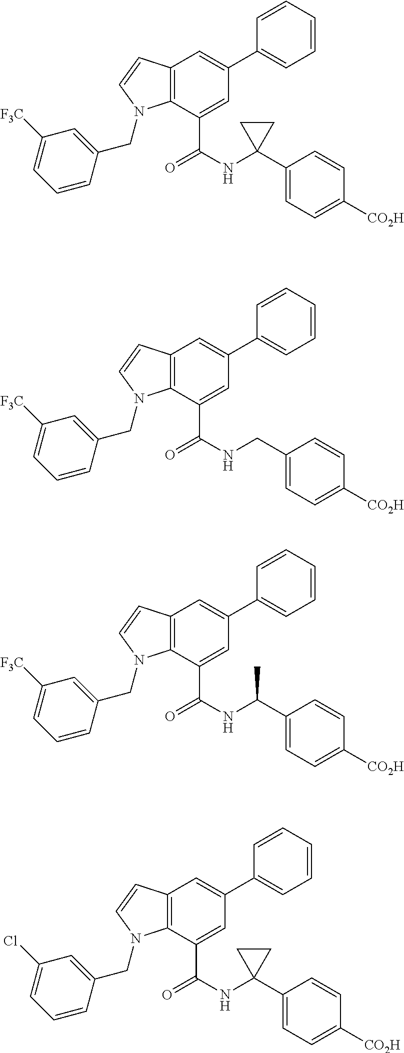 [SMILES: O=C(O)c1ccc(C2(NC(=O)c3cc(-c4ccccc4)cc4ccn(Cc5cccc(C(F)(F)F)c5)c34)CC2)cc1 ; O=C(O)c1ccc(CNC(=O)c2cc(-c3ccccc3)cc3ccn(Cc4cccc(C(F)(F)F)c4)c23)cc1 ; C[C@H](NC(=O)c1cc(-c2ccccc2)cc2ccn(Cc3cccc(C(F)(F)F)c3)c12)c1ccc(C(=O)O)cc1 ; O=C(O)c1ccc(C2(NC(=O)c3cc(-c4ccccc4)cc4ccn(Cc5cccc(Cl)c5)c34)CC2)cc1]

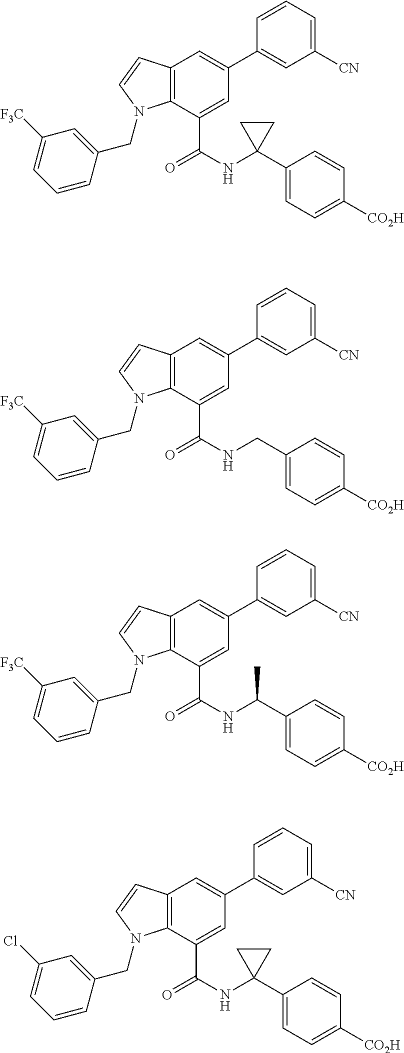 [SMILES: N#Cc1cccc(-c2cc(C(=O)NC3(c4ccc(C(=O)O)cc4)CC3)c3c(ccn3Cc3cccc(C(F)(F)F)c3)c2)c1 ; N#Cc1cccc(-c2cc(C(=O)NCc3ccc(C(=O)O)cc3)c3c(ccn3Cc3cccc(C(F)(F)F)c3)c2)c1 ; C[C@H](NC(=O)c1cc(-c2cccc(C#N)c2)cc2ccn(Cc3cccc(C(F)(F)F)c3)c12)c1ccc(C(=O)O)cc1 ; N#Cc1cccc(-c2cc(C(=O)NC3(c4ccc(C(=O)O)cc4)CC3)c3c(ccn3Cc3cccc(Cl)c3)c2)c1]

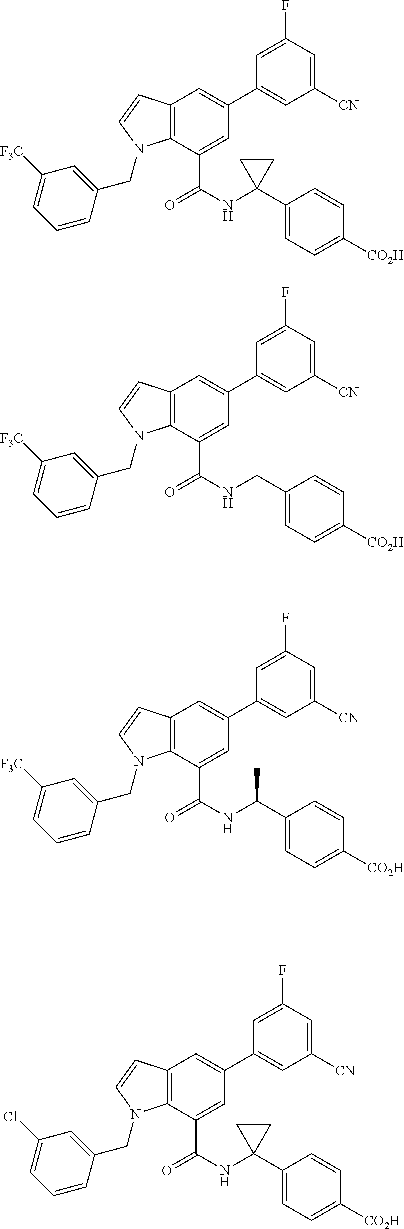 [SMILES: N#Cc1cc(F)cc(-c2cc(C(=O)NC3(c4ccc(C(=O)O)cc4)CC3)c3c(ccn3Cc3cccc(C(F)(F)F)c3)c2)c1 ; N#Cc1cc(F)cc(-c2cc(C(=O)NCc3ccc(C(=O)O)cc3)c3c(ccn3Cc3cccc(C(F)(F)F)c3)c2)c1 ; C[C@H](NC(=O)c1cc(-c2cc(F)cc(C#N)c2)cc2ccn(Cc3cccc(C(F)(F)F)c3)c12)c1ccc(C(=O)O)cc1 ; N#Cc1cc(F)cc(-c2cc(C(=O)NC3(c4ccc(C(=O)O)cc4)CC3)c3c(ccn3Cc3cccc(Cl)c3)c2)c1]

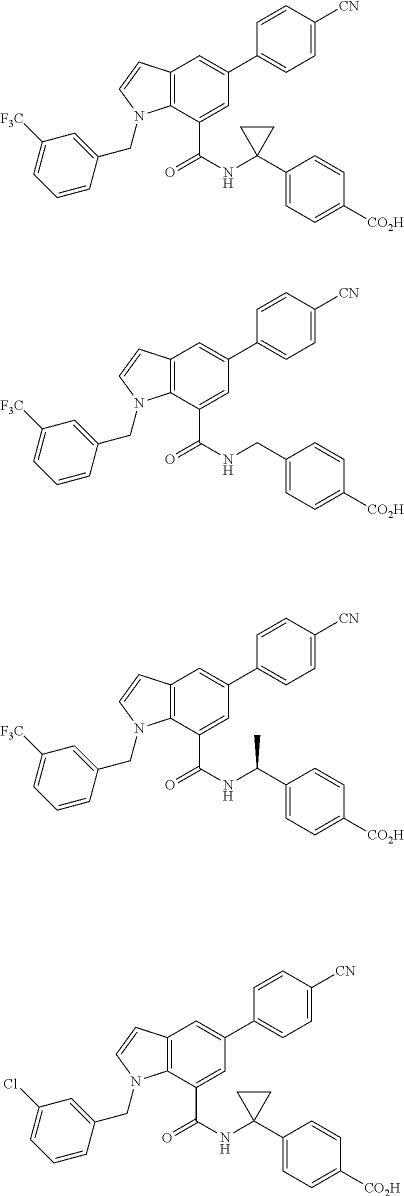 [SMILES: N#Cc1ccc(-c2cc(C(=O)NC3(c4ccc(C(=O)O)cc4)CC3)c3c(ccn3Cc3cccc(C(F)(F)F)c3)c2)cc1 ; N#Cc1ccc(-c2cc(C(=O)NCc3ccc(C(=O)O)cc3)c3c(ccn3Cc3cccc(C(F)(F)F)c3)c2)cc1 ; C[C@H](NC(=O)c1cc(-c2ccc(C#N)cc2)cc2ccn(Cc3cccc(C(F)(F)F)c3)c12)c1ccc(C(=O)O)cc1 ; N#Cc1ccc(-c2cc(C(=O)NC3(c4ccc(C(=O)O)cc4)CC3)c3c(ccn3Cc3cccc(Cl)c3)c2)cc1]

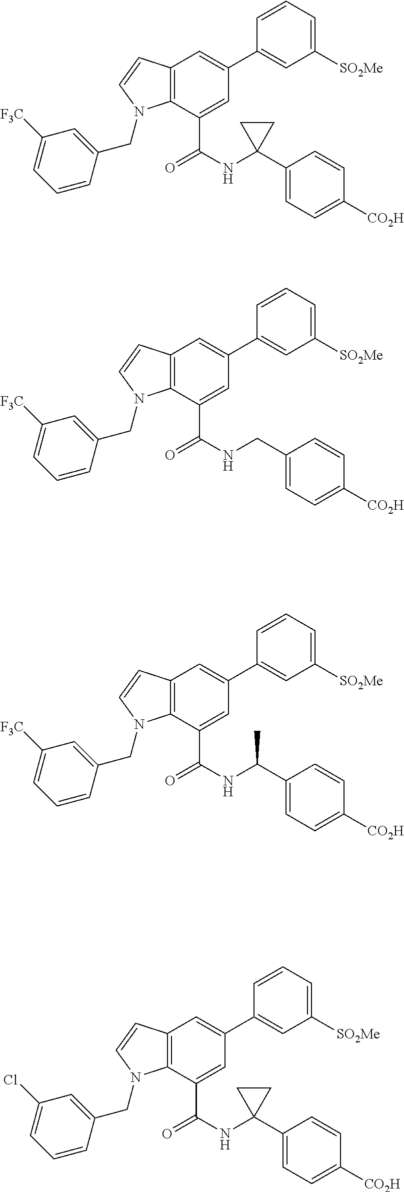 [SMILES: CS(=O)(=O)c1cccc(-c2cc(C(=O)NC3(c4ccc(C(=O)O)cc4)CC3)c3c(ccn3Cc3cccc(C(F)(F)F)c3)c2)c1 ; CS(=O)(=O)c1cccc(-c2cc(C(=O)NCc3ccc(C(=O)O)cc3)c3c(ccn3Cc3cccc(C(F)(F)F)c3)c2)c1 ; C[C@H](NC(=O)c1cc(-c2cccc(S(C)(=O)=O)c2)cc2ccn(Cc3cccc(C(F)(F)F)c3)c12)c1ccc(C(=O)O)cc1 ; CS(=O)(=O)c1cccc(-c2cc(C(=O)NC3(c4ccc(C(=O)O)cc4)CC3)c3c(ccn3Cc3cccc(Cl)c3)c2)c1]

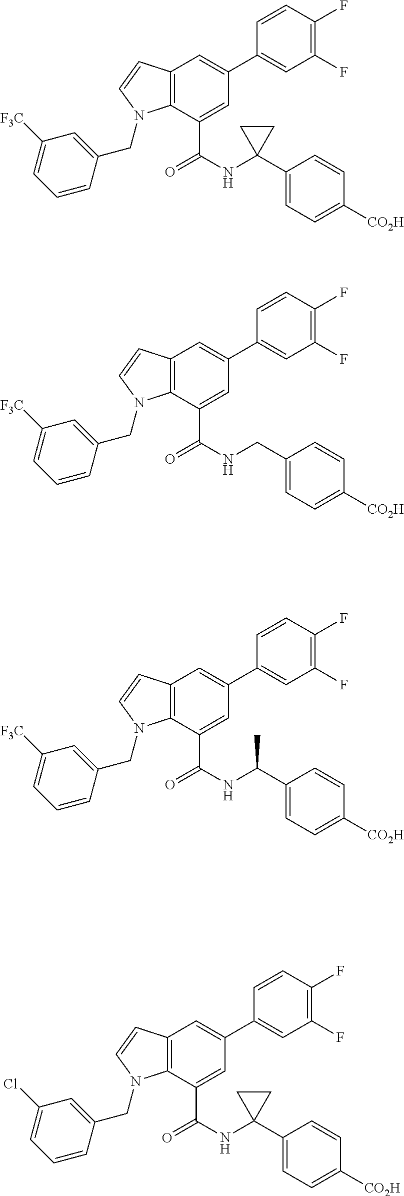 [SMILES: O=C(O)c1ccc(C2(NC(=O)c3cc(-c4ccc(F)c(F)c4)cc4ccn(Cc5cccc(C(F)(F)F)c5)c34)CC2)cc1 ; O=C(O)c1ccc(CNC(=O)c2cc(-c3ccc(F)c(F)c3)cc3ccn(Cc4cccc(C(F)(F)F)c4)c23)cc1 ; C[C@H](NC(=O)c1cc(-c2ccc(F)c(F)c2)cc2ccn(Cc3cccc(C(F)(F)F)c3)c12)c1ccc(C(=O)O)cc1 ; O=C(O)c1ccc(C2(NC(=O)c3cc(-c4ccc(F)c(F)c4)cc4ccn(Cc5cccc(Cl)c5)c34)CC2)cc1]

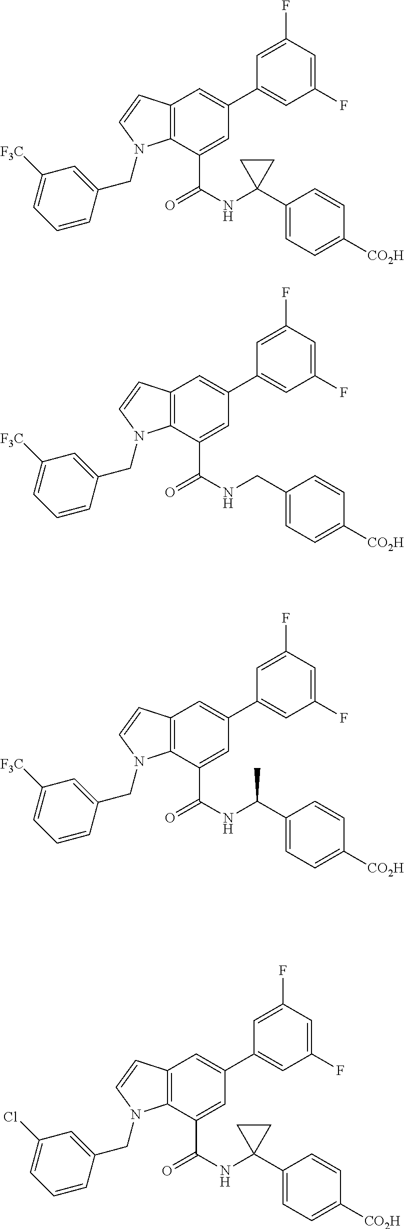 [SMILES: O=C(O)c1ccc(C2(NC(=O)c3cc(-c4cc(F)cc(F)c4)cc4ccn(Cc5cccc(C(F)(F)F)c5)c34)CC2)cc1 ; O=C(O)c1ccc(CNC(=O)c2cc(-c3cc(F)cc(F)c3)cc3ccn(Cc4cccc(C(F)(F)F)c4)c23)cc1 ; C[C@H](NC(=O)c1cc(-c2cc(F)cc(F)c2)cc2ccn(Cc3cccc(C(F)(F)F)c3)c12)c1ccc(C(=O)O)cc1 ; O=C(O)c1ccc(C2(NC(=O)c3cc(-c4cc(F)cc(F)c4)cc4ccn(Cc5cccc(Cl)c5)c34)CC2)cc1]

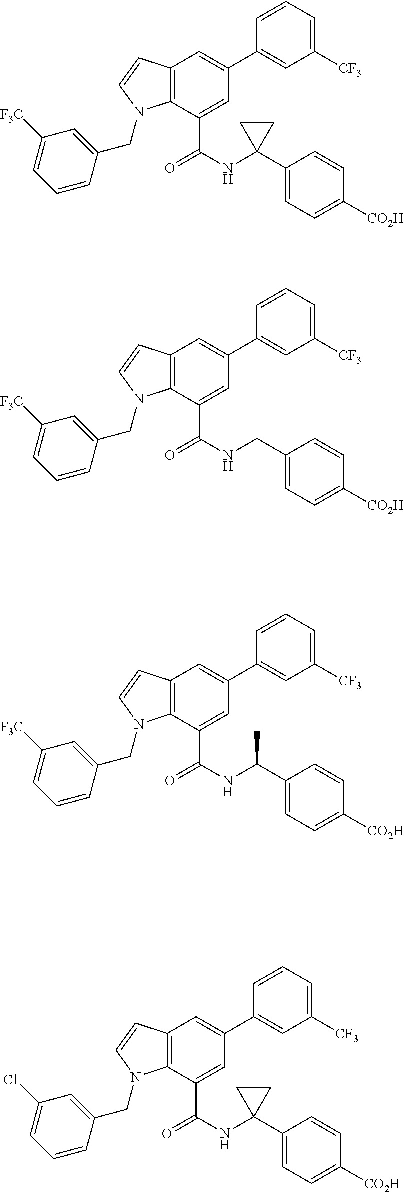 [SMILES: O=C(O)c1ccc(C2(NC(=O)c3cc(-c4cccc(C(F)(F)F)c4)cc4ccn(Cc5cccc(C(F)(F)F)c5)c34)CC2)cc1 ; O=C(O)c1ccc(CNC(=O)c2cc(-c3cccc(C(F)(F)F)c3)cc3ccn(Cc4cccc(C(F)(F)F)c4)c23)cc1 ; C[C@H](NC(=O)c1cc(-c2cccc(C(F)(F)F)c2)cc2ccn(Cc3cccc(C(F)(F)F)c3)c12)c1ccc(C(=O)O)cc1 ; O=C(O)c1ccc(C2(NC(=O)c3cc(-c4cccc(C(F)(F)F)c4)cc4ccn(Cc5cccc(Cl)c5)c34)CC2)cc1]

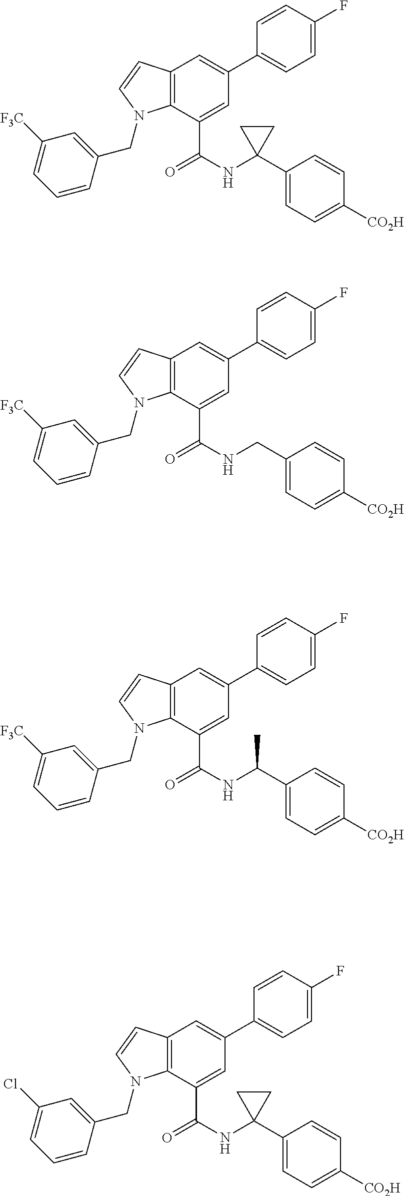 [SMILES: O=C(O)c1ccc(C2(NC(=O)c3cc(-c4ccc(F)cc4)cc4ccn(Cc5cccc(C(F)(F)F)c5)c34)CC2)cc1 ; O=C(O)c1ccc(CNC(=O)c2cc(-c3ccc(F)cc3)cc3ccn(Cc4cccc(C(F)(F)F)c4)c23)cc1 ; C[C@H](NC(=O)c1cc(-c2ccc(F)cc2)cc2ccn(Cc3cccc(C(F)(F)F)c3)c12)c1ccc(C(=O)O)cc1 ; O=C(O)c1ccc(C2(NC(=O)c3cc(-c4ccc(F)cc4)cc4ccn(Cc5cccc(Cl)c5)c34)CC2)cc1]

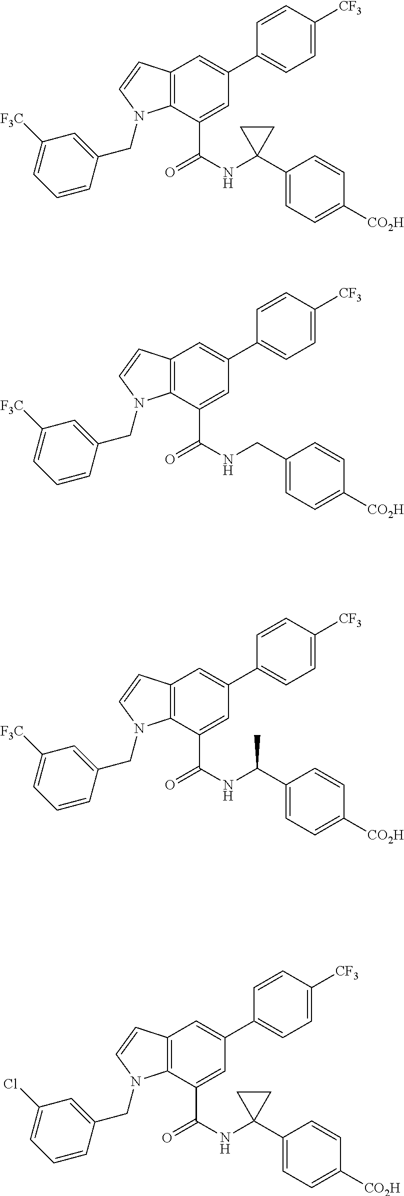 [SMILES: O=C(O)c1ccc(C2(NC(=O)c3cc(-c4ccc(C(F)(F)F)cc4)cc4ccn(Cc5cccc(C(F)(F)F)c5)c34)CC2)cc1 ; O=C(O)c1ccc(CNC(=O)c2cc(-c3ccc(C(F)(F)F)cc3)cc3ccn(Cc4cccc(C(F)(F)F)c4)c23)cc1 ; C[C@H](NC(=O)c1cc(-c2ccc(C(F)(F)F)cc2)cc2ccn(Cc3cccc(C(F)(F)F)c3)c12)c1ccc(C(=O)O)cc1 ; O=C(O)c1ccc(C2(NC(=O)c3cc(-c4ccc(C(F)(F)F)cc4)cc4ccn(Cc5cccc(Cl)c5)c34)CC2)cc1]

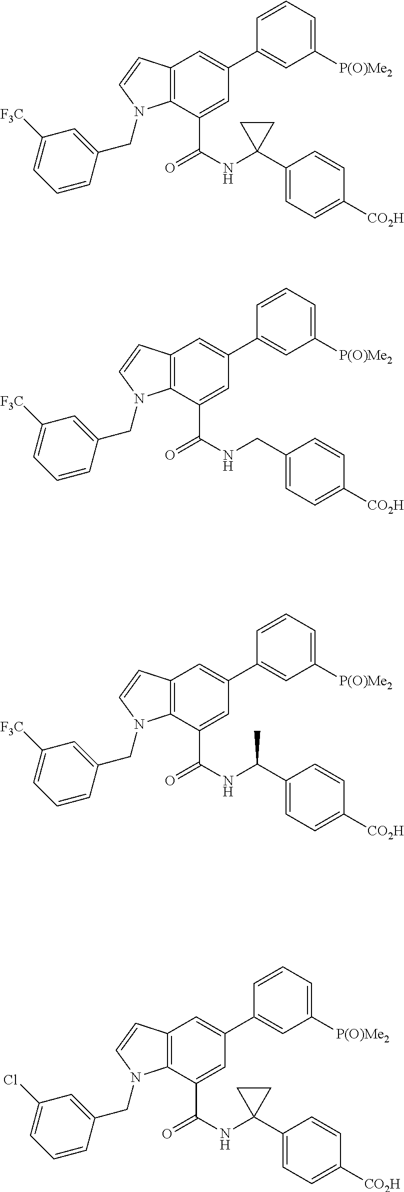

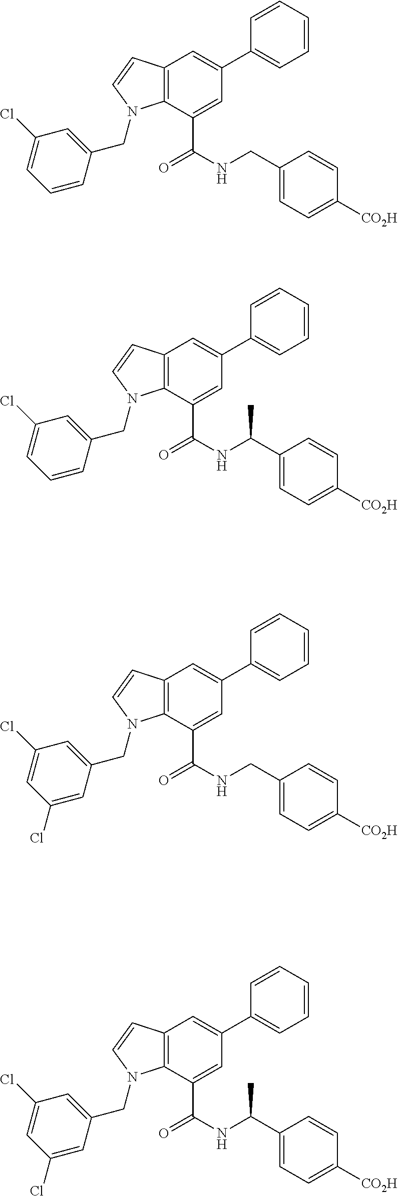 [SMILES: O=C(O)c1ccc(CNC(=O)c2cc(-c3ccccc3)cc3ccn(Cc4cccc(Cl)c4)c23)cc1 ; C[C@H](NC(=O)c1cc(-c2ccccc2)cc2ccn(Cc3cccc(Cl)c3)c12)c1ccc(C(=O)O)cc1 ; O=C(O)c1ccc(CNC(=O)c2cc(-c3ccccc3)cc3ccn(Cc4cc(Cl)cc(Cl)c4)c23)cc1 ; C[C@H](NC(=O)c1cc(-c2ccccc2)cc2ccn(Cc3cc(Cl)cc(Cl)c3)c12)c1ccc(C(=O)O)cc1]

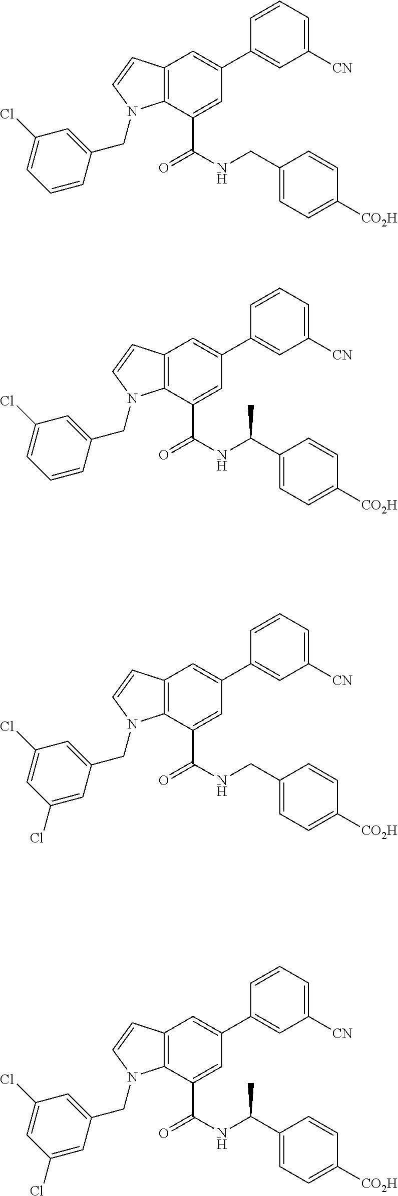 [SMILES: N#Cc1cccc(-c2cc(C(=O)NCc3ccc(C(=O)O)cc3)c3c(ccn3Cc3cccc(Cl)c3)c2)c1 ; C[C@H](NC(=O)c1cc(-c2cccc(C#N)c2)cc2ccn(Cc3cccc(Cl)c3)c12)c1ccc(C(=O)O)cc1 ; N#Cc1cccc(-c2cc(C(=O)NCc3ccc(C(=O)O)cc3)c3c(ccn3Cc3cc(Cl)cc(Cl)c3)c2)c1 ; C[C@H](NC(=O)c1cc(-c2cccc(C#N)c2)cc2ccn(Cc3cc(Cl)cc(Cl)c3)c12)c1ccc(C(=O)O)cc1]

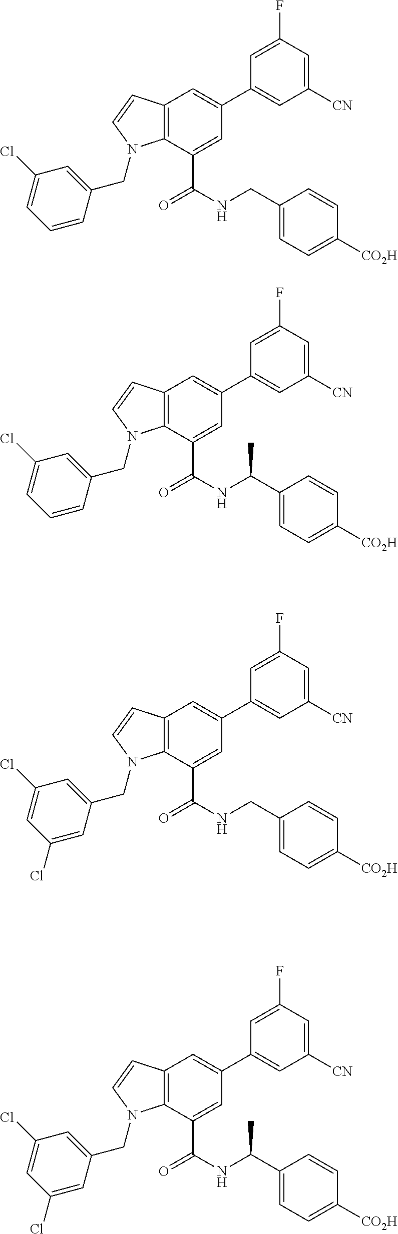 [SMILES: N#Cc1cc(F)cc(-c2cc(C(=O)NCc3ccc(C(=O)O)cc3)c3c(ccn3Cc3cccc(Cl)c3)c2)c1 ; C[C@H](NC(=O)c1cc(-c2cc(F)cc(C#N)c2)cc2ccn(Cc3cccc(Cl)c3)c12)c1ccc(C(=O)O)cc1 ; N#Cc1cc(F)cc(-c2cc(C(=O)NCc3ccc(C(=O)O)cc3)c3c(ccn3Cc3cc(Cl)cc(Cl)c3)c2)c1 ; C[C@H](NC(=O)c1cc(-c2cc(F)cc(C#N)c2)cc2ccn(Cc3cc(Cl)cc(Cl)c3)c12)c1ccc(C(=O)O)cc1]

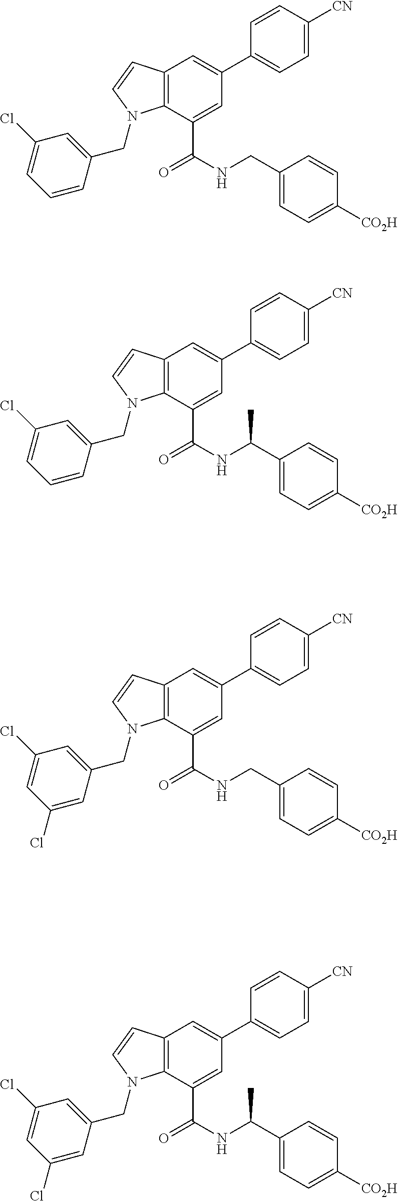 [SMILES: N#Cc1ccc(-c2cc(C(=O)NCc3ccc(C(=O)O)cc3)c3c(ccn3Cc3cccc(Cl)c3)c2)cc1 ; C[C@H](NC(=O)c1cc(-c2ccc(C#N)cc2)cc2ccn(Cc3cccc(Cl)c3)c12)c1ccc(C(=O)O)cc1 ; N#Cc1ccc(-c2cc(C(=O)NCc3ccc(C(=O)O)cc3)c3c(ccn3Cc3cc(Cl)cc(Cl)c3)c2)cc1 ; C[C@H](NC(=O)c1cc(-c2ccc(C#N)cc2)cc2ccn(Cc3cc(Cl)cc(Cl)c3)c12)c1ccc(C(=O)O)cc1]

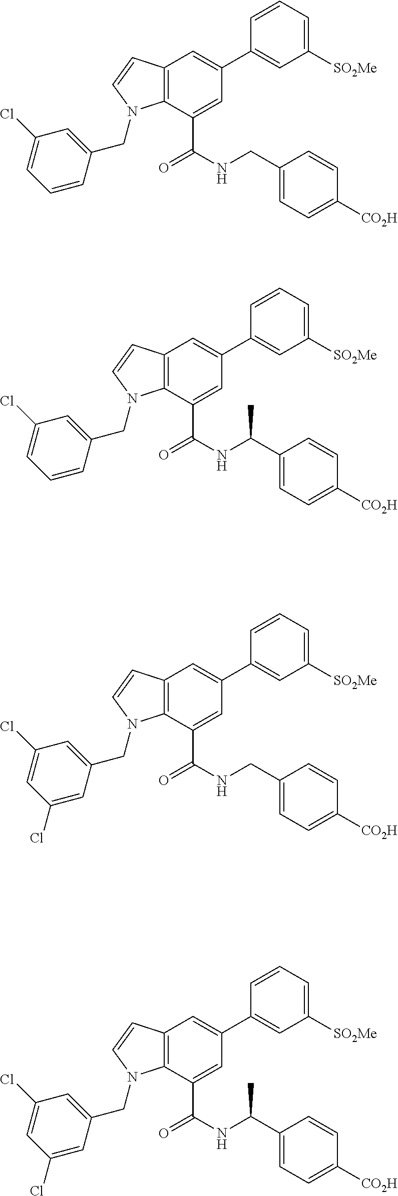 [SMILES: CS(=O)(=O)c1cccc(-c2cc(C(=O)NCc3ccc(C(=O)O)cc3)c3c(ccn3Cc3cccc(Cl)c3)c2)c1 ; C[C@H](NC(=O)c1cc(-c2cccc(S(C)(=O)=O)c2)cc2ccn(Cc3cccc(Cl)c3)c12)c1ccc(C(=O)O)cc1 ; CS(=O)(=O)c1cccc(-c2cc(C(=O)NCc3ccc(C(=O)O)cc3)c3c(ccn3Cc3cc(Cl)cc(Cl)c3)c2)c1 ; C[C@H](NC(=O)c1cc(-c2cccc(S(C)(=O)=O)c2)cc2ccn(Cc3cc(Cl)cc(Cl)c3)c12)c1ccc(C(=O)O)cc1]

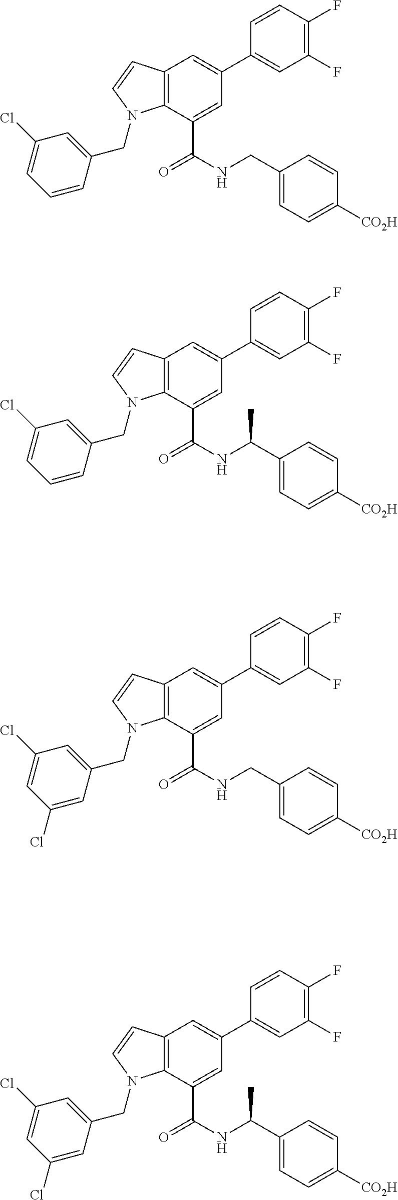 [SMILES: O=C(O)c1ccc(CNC(=O)c2cc(-c3ccc(F)c(F)c3)cc3ccn(Cc4cccc(Cl)c4)c23)cc1 ; C[C@H](NC(=O)c1cc(-c2ccc(F)c(F)c2)cc2ccn(Cc3cccc(Cl)c3)c12)c1ccc(C(=O)O)cc1 ; O=C(O)c1ccc(CNC(=O)c2cc(-c3ccc(F)c(F)c3)cc3ccn(Cc4cc(Cl)cc(Cl)c4)c23)cc1 ; C[C@H](NC(=O)c1cc(-c2ccc(F)c(F)c2)cc2ccn(Cc3cc(Cl)cc(Cl)c3)c12)c1ccc(C(=O)O)cc1]

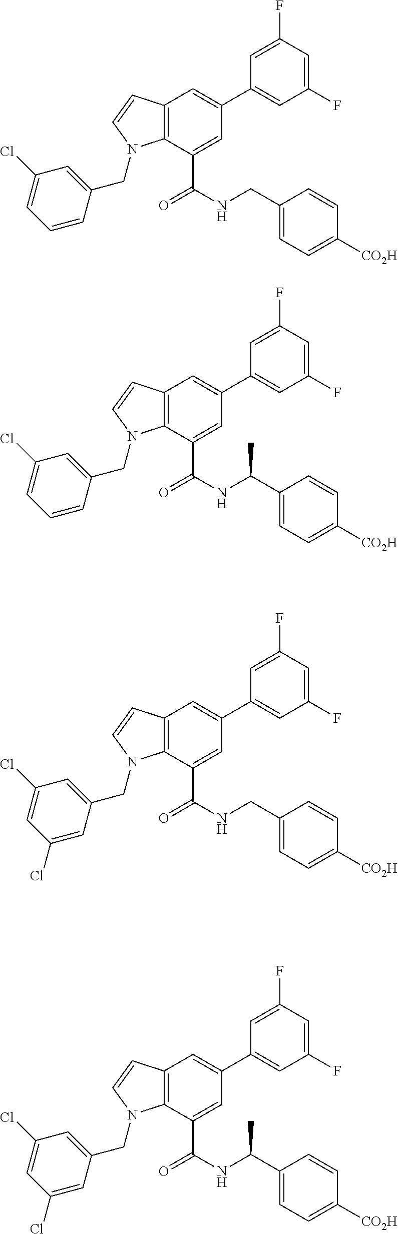 [SMILES: O=C(O)c1ccc(CNC(=O)c2cc(-c3cc(F)cc(F)c3)cc3ccn(Cc4cccc(Cl)c4)c23)cc1 ; C[C@H](NC(=O)c1cc(-c2cc(F)cc(F)c2)cc2ccn(Cc3cccc(Cl)c3)c12)c1ccc(C(=O)O)cc1 ; O=C(O)c1ccc(CNC(=O)c2cc(-c3cc(F)cc(F)c3)cc3ccn(Cc4cc(Cl)cc(Cl)c4)c23)cc1 ; C[C@H](NC(=O)c1cc(-c2cc(F)cc(F)c2)cc2ccn(Cc3cc(Cl)cc(Cl)c3)c12)c1ccc(C(=O)O)cc1]

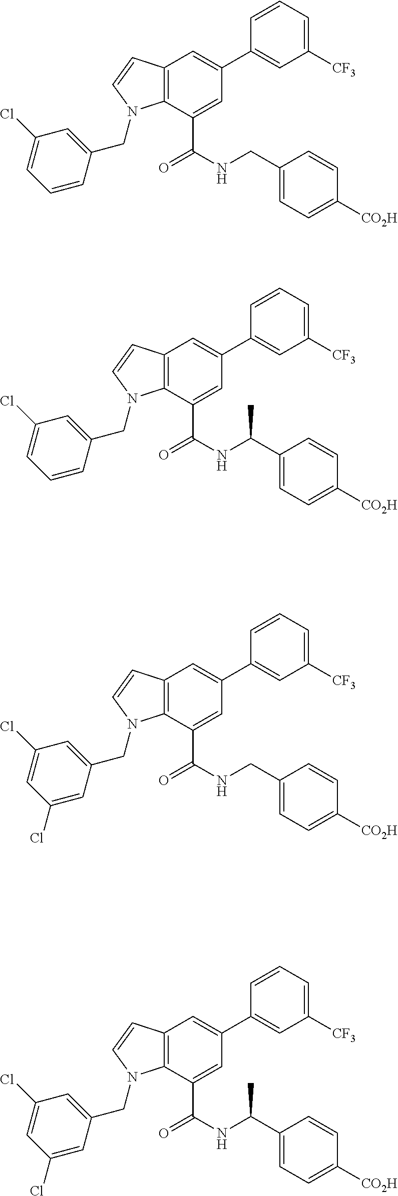 [SMILES: O=C(O)c1ccc(CNC(=O)c2cc(-c3cccc(C(F)(F)F)c3)cc3ccn(Cc4cccc(Cl)c4)c23)cc1 ; C[C@H](NC(=O)c1cc(-c2cccc(C(F)(F)F)c2)cc2ccn(Cc3cccc(Cl)c3)c12)c1ccc(C(=O)O)cc1 ; O=C(O)c1ccc(CNC(=O)c2cc(-c3cccc(C(F)(F)F)c3)cc3ccn(Cc4cc(Cl)cc(Cl)c4)c23)cc1 ; C[C@H](NC(=O)c1cc(-c2cccc(C(F)(F)F)c2)cc2ccn(Cc3cc(Cl)cc(Cl)c3)c12)c1ccc(C(=O)O)cc1]

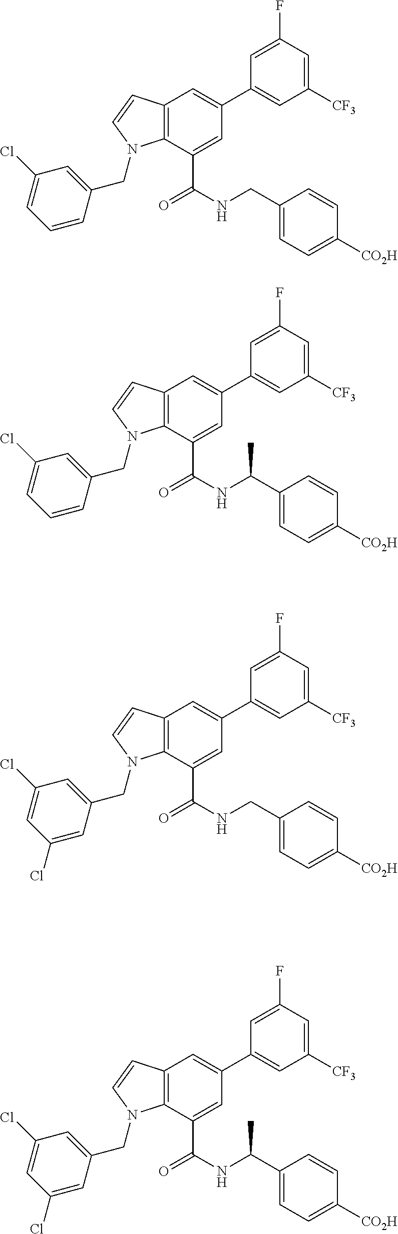 [SMILES: O=C(O)c1ccc(CNC(=O)c2cc(-c3cc(F)cc(C(F)(F)F)c3)cc3ccn(Cc4cccc(Cl)c4)c23)cc1 ; C[C@H](NC(=O)c1cc(-c2cc(F)cc(C(F)(F)F)c2)cc2ccn(Cc3cccc(Cl)c3)c12)c1ccc(C(=O)O)cc1 ; O=C(O)c1ccc(CNC(=O)c2cc(-c3cc(F)cc(C(F)(F)F)c3)cc3ccn(Cc4cc(Cl)cc(Cl)c4)c23)cc1 ; C[C@H](NC(=O)c1cc(-c2cc(F)cc(C(F)(F)F)c2)cc2ccn(Cc3cc(Cl)cc(Cl)c3)c12)c1ccc(C(=O)O)cc1]

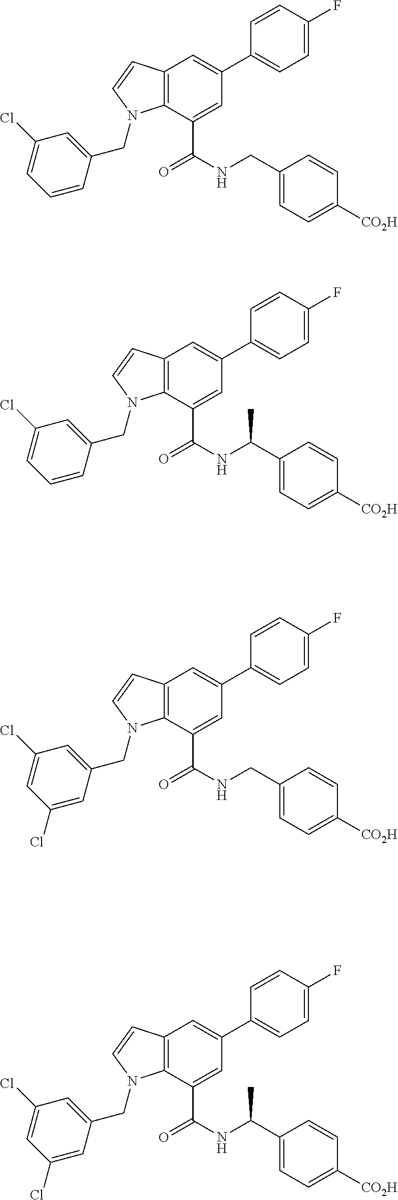 [SMILES: O=C(O)c1ccc(CNC(=O)c2cc(-c3ccc(F)cc3)cc3ccn(Cc4cccc(Cl)c4)c23)cc1 ; C[C@H](NC(=O)c1cc(-c2ccc(F)cc2)cc2ccn(Cc3cccc(Cl)c3)c12)c1ccc(C(=O)O)cc1 ; O=C(O)c1ccc(CNC(=O)c2cc(-c3ccc(F)cc3)cc3ccn(Cc4cc(Cl)cc(Cl)c4)c23)cc1 ; C[C@H](NC(=O)c1cc(-c2ccc(F)cc2)cc2ccn(Cc3cc(Cl)cc(Cl)c3)c12)c1ccc(C(=O)O)cc1]

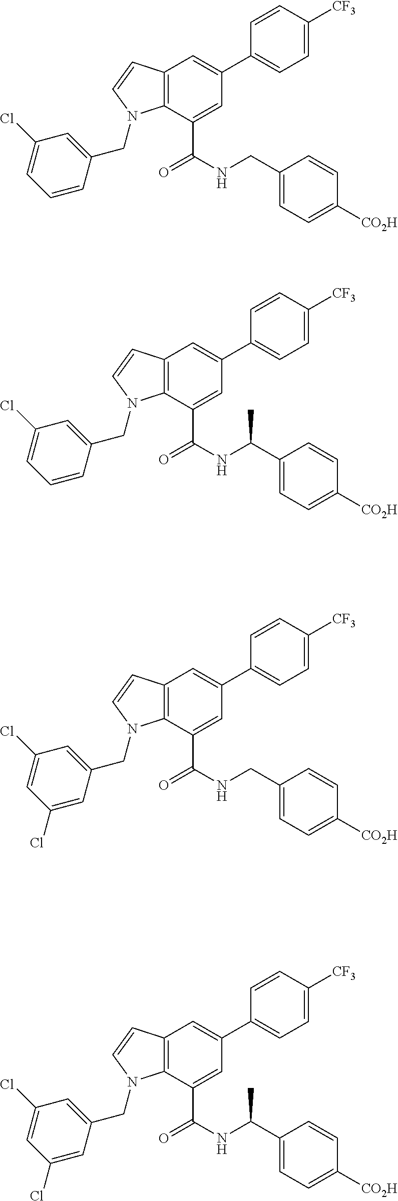 [SMILES: O=C(O)c1ccc(CNC(=O)c2cc(-c3ccc(C(F)(F)F)cc3)cc3ccn(Cc4cccc(Cl)c4)c23)cc1 ; C[C@H](NC(=O)c1cc(-c2ccc(C(F)(F)F)cc2)cc2ccn(Cc3cccc(Cl)c3)c12)c1ccc(C(=O)O)cc1 ; O=C(O)c1ccc(CNC(=O)c2cc(-c3ccc(C(F)(F)F)cc3)cc3ccn(Cc4cc(Cl)cc(Cl)c4)c23)cc1 ; C[C@H](NC(=O)c1cc(-c2ccc(C(F)(F)F)cc2)cc2ccn(Cc3cc(Cl)cc(Cl)c3)c12)c1ccc(C(=O)O)cc1]

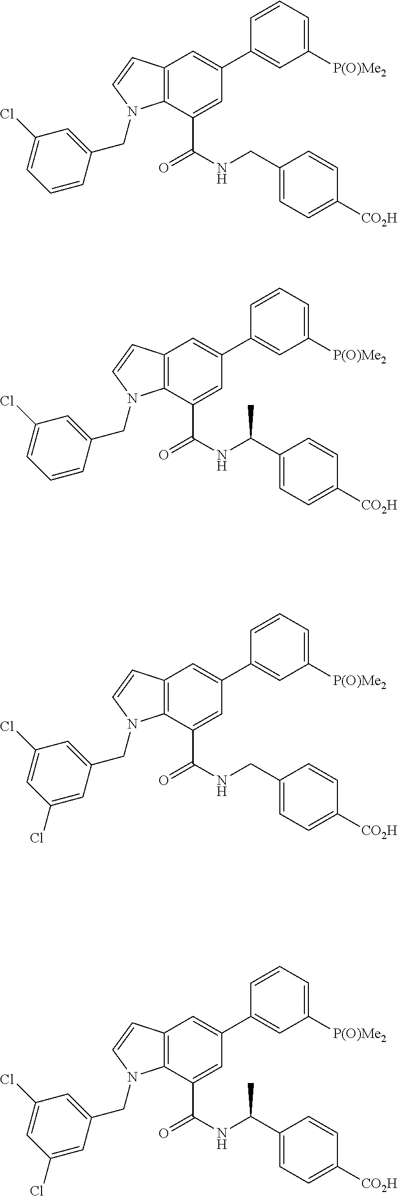

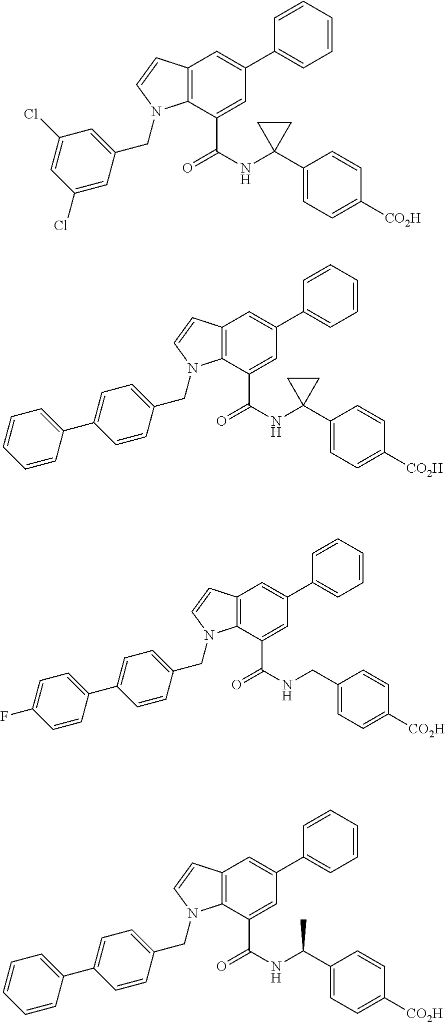 [SMILES: O=C(O)c1ccc(C2(NC(=O)c3cc(-c4ccccc4)cc4ccn(Cc5cc(Cl)cc(Cl)c5)c34)CC2)cc1 ; O=C(O)c1ccc(C2(NC(=O)c3cc(-c4ccccc4)cc4ccn(Cc5ccc(-c6ccccc6)cc5)c34)CC2)cc1 ; O=C(O)c1ccc(CNC(=O)c2cc(-c3ccccc3)cc3ccn(Cc4ccc(-c5ccc(F)cc5)cc4)c23)cc1 ; C[C@H](NC(=O)c1cc(-c2ccccc2)cc2ccn(Cc3ccc(-c4ccccc4)cc3)c12)c1ccc(C(=O)O)cc1]

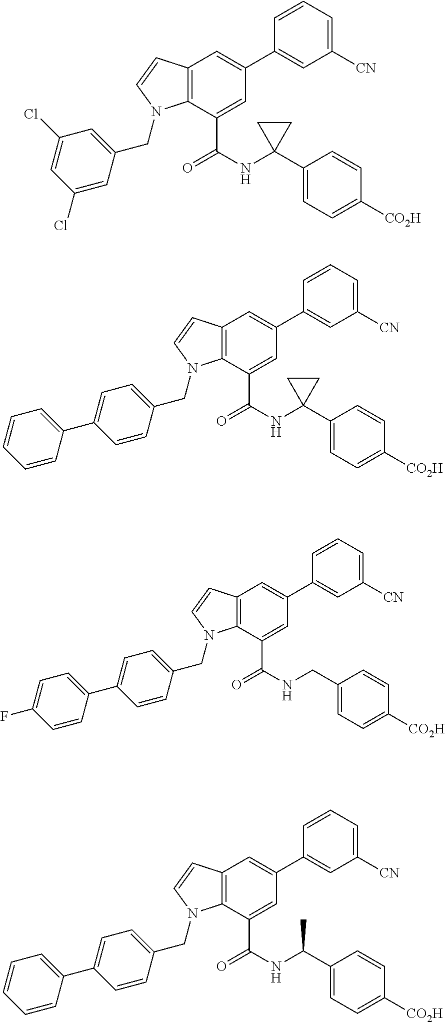 [SMILES: N#Cc1cccc(-c2cc(C(=O)NC3(c4ccc(C(=O)O)cc4)CC3)c3c(ccn3Cc3cc(Cl)cc(Cl)c3)c2)c1 ; N#Cc1cccc(-c2cc(C(=O)NC3(c4ccc(C(=O)O)cc4)CC3)c3c(ccn3Cc3ccc(-c4ccccc4)cc3)c2)c1 ; N#Cc1cccc(-c2cc(C(=O)NCc3ccc(C(=O)O)cc3)c3c(ccn3Cc3ccc(-c4ccc(F)cc4)cc3)c2)c1 ; C[C@H](NC(=O)c1cc(-c2cccc(C#N)c2)cc2ccn(Cc3ccc(-c4ccccc4)cc3)c12)c1ccc(C(=O)O)cc1]

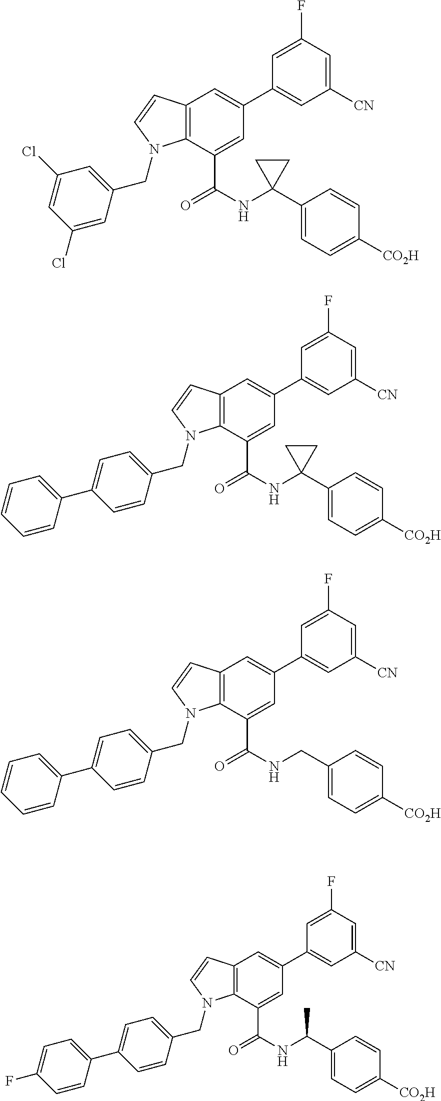 [SMILES: N#Cc1cc(F)cc(-c2cc(C(=O)NC3(c4ccc(C(=O)O)cc4)CC3)c3c(ccn3Cc3cc(Cl)cc(Cl)c3)c2)c1 ; N#Cc1cc(F)cc(-c2cc(C(=O)NC3(c4ccc(C(=O)O)cc4)CC3)c3c(ccn3Cc3ccc(-c4ccccc4)cc3)c2)c1 ; N#Cc1cc(F)cc(-c2cc(C(=O)NCc3ccc(C(=O)O)cc3)c3c(ccn3Cc3ccc(-c4ccccc4)cc3)c2)c1 ; C[C@H](NC(=O)c1cc(-c2cc(F)cc(C#N)c2)cc2ccn(Cc3ccc(-c4ccc(F)cc4)cc3)c12)c1ccc(C(=O)O)cc1]

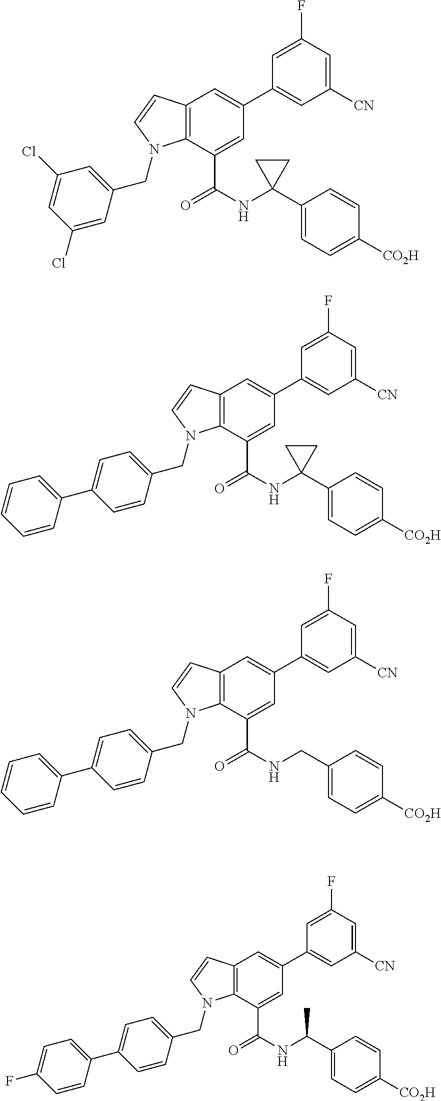 [SMILES: N#Cc1cc(F)cc(-c2cc(C(=O)NC3(c4ccc(C(=O)O)cc4)CC3)c3c(ccn3Cc3cc(Cl)cc(Cl)c3)c2)c1 ; N#Cc1cc(F)cc(-c2cc(C(=O)NC3(c4ccc(C(=O)O)cc4)CC3)c3c(ccn3Cc3ccc(-c4ccccc4)cc3)c2)c1 ; N#Cc1cc(F)cc(-c2cc(C(=O)NCc3ccc(C(=O)O)cc3)c3c(ccn3Cc3ccc(-c4ccccc4)cc3)c2)c1 ; C[C@H](NC(=O)c1cc(-c2cc(F)cc(C#N)c2)cc2ccn(Cc3ccc(-c4ccc(F)cc4)cc3)c12)c1ccc(C(=O)O)cc1]

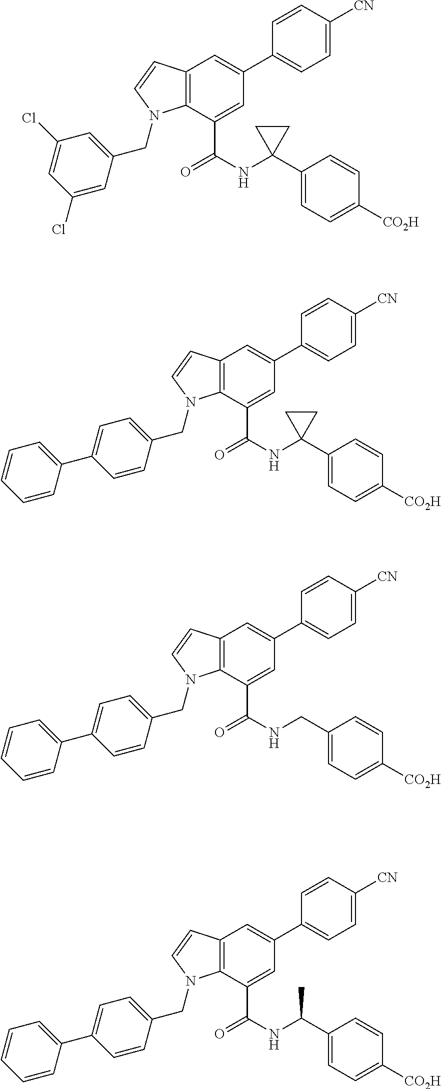 [SMILES: N#Cc1ccc(-c2cc(C(=O)NC3(c4ccc(C(=O)O)cc4)CC3)c3c(ccn3Cc3cc(Cl)cc(Cl)c3)c2)cc1 ; N#Cc1ccc(-c2cc(C(=O)NC3(c4ccc(C(=O)O)cc4)CC3)c3c(ccn3Cc3ccc(-c4ccccc4)cc3)c2)cc1 ; N#Cc1ccc(-c2cc(C(=O)NCc3ccc(C(=O)O)cc3)c3c(ccn3Cc3ccc(-c4ccccc4)cc3)c2)cc1 ; C[C@H](NC(=O)c1cc(-c2ccc(C#N)cc2)cc2ccn(Cc3ccc(-c4ccccc4)cc3)c12)c1ccc(C(=O)O)cc1]

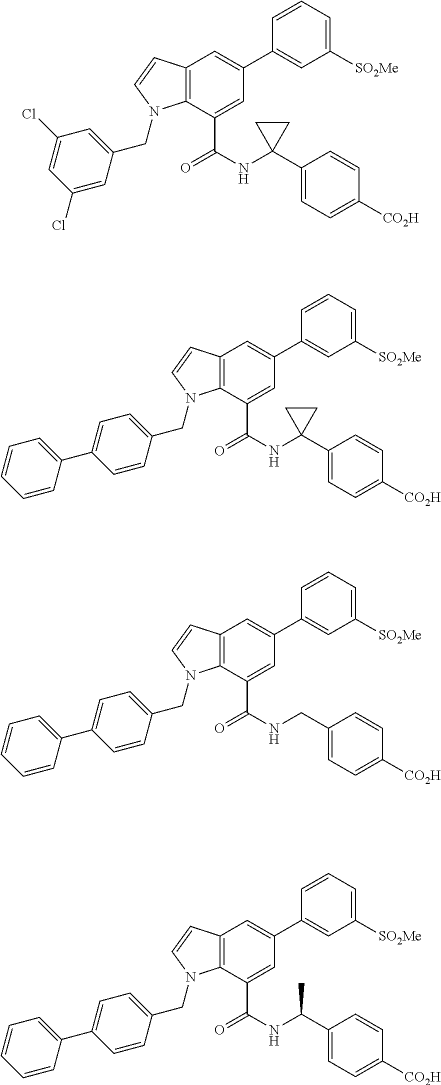 [SMILES: CS(=O)(=O)c1cccc(-c2cc(C(=O)NC3(c4ccc(C(=O)O)cc4)CC3)c3c(ccn3Cc3cc(Cl)cc(Cl)c3)c2)c1 ; CS(=O)(=O)c1cccc(-c2cc(C(=O)NC3(c4ccc(C(=O)O)cc4)CC3)c3c(ccn3Cc3ccc(-c4ccccc4)cc3)c2)c1 ; CS(=O)(=O)c1cccc(-c2cc(C(=O)NCc3ccc(C(=O)O)cc3)c3c(ccn3Cc3ccc(-c4ccccc4)cc3)c2)c1 ; C[C@H](NC(=O)c1cc(-c2cccc(S(C)(=O)=O)c2)cc2ccn(Cc3ccc(-c4ccccc4)cc3)c12)c1ccc(C(=O)O)cc1]

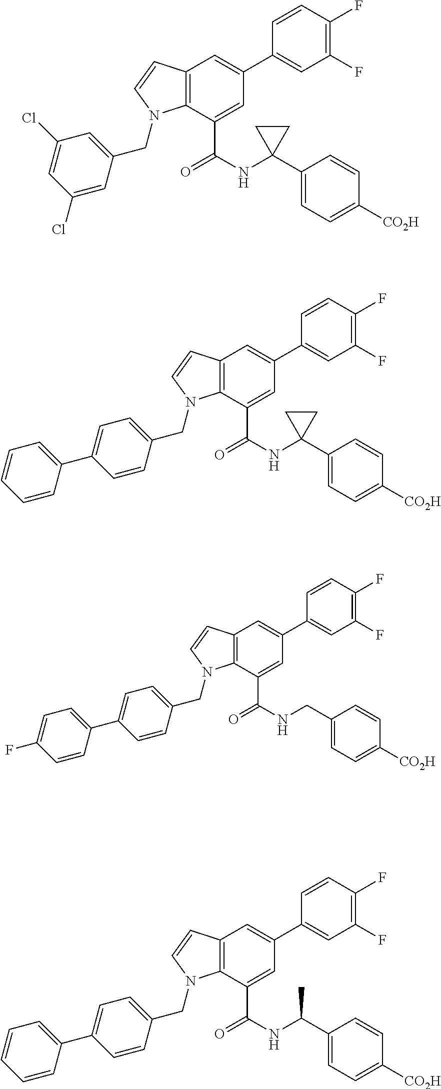 [SMILES: O=C(O)c1ccc(C2(NC(=O)c3cc(-c4ccc(F)c(F)c4)cc4ccn(Cc5cc(Cl)cc(Cl)c5)c34)CC2)cc1 ; O=C(O)c1ccc(C2(NC(=O)c3cc(-c4ccc(F)c(F)c4)cc4ccn(Cc5ccc(-c6ccccc6)cc5)c34)CC2)cc1 ; O=C(O)c1ccc(CNC(=O)c2cc(-c3ccc(F)c(F)c3)cc3ccn(Cc4ccc(-c5ccc(F)cc5)cc4)c23)cc1 ; C[C@H](NC(=O)c1cc(-c2ccc(F)c(F)c2)cc2ccn(Cc3ccc(-c4ccccc4)cc3)c12)c1ccc(C(=O)O)cc1]

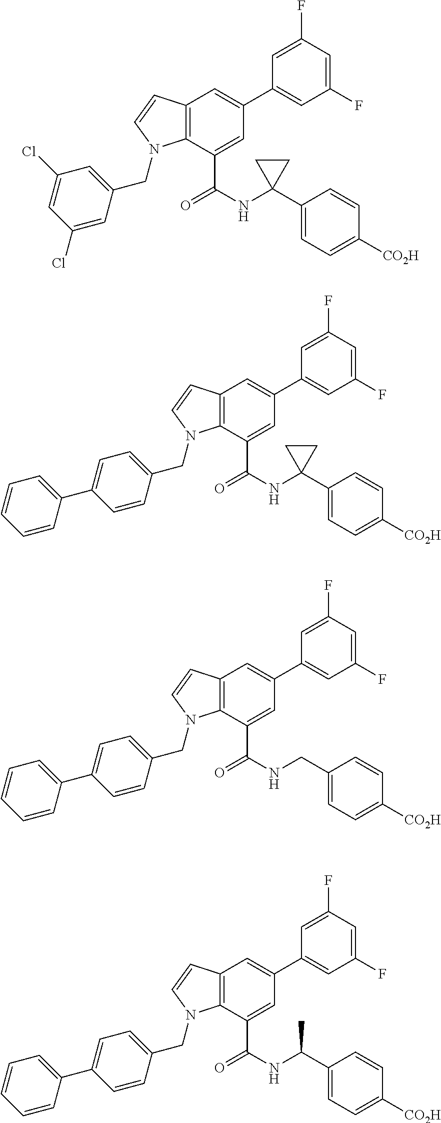 [SMILES: O=C(O)c1ccc(C2(NC(=O)c3cc(-c4cc(F)cc(F)c4)cc4ccn(Cc5cc(Cl)cc(Cl)c5)c34)CC2)cc1 ; O=C(O)c1ccc(C2(NC(=O)c3cc(-c4cc(F)cc(F)c4)cc4ccn(Cc5ccc(-c6ccccc6)cc5)c34)CC2)cc1 ; O=C(O)c1ccc(CNC(=O)c2cc(-c3cc(F)cc(F)c3)cc3ccn(Cc4ccc(-c5ccccc5)cc4)c23)cc1 ; C[C@H](NC(=O)c1cc(-c2cc(F)cc(F)c2)cc2ccn(Cc3ccc(-c4ccccc4)cc3)c12)c1ccc(C(=O)O)cc1]

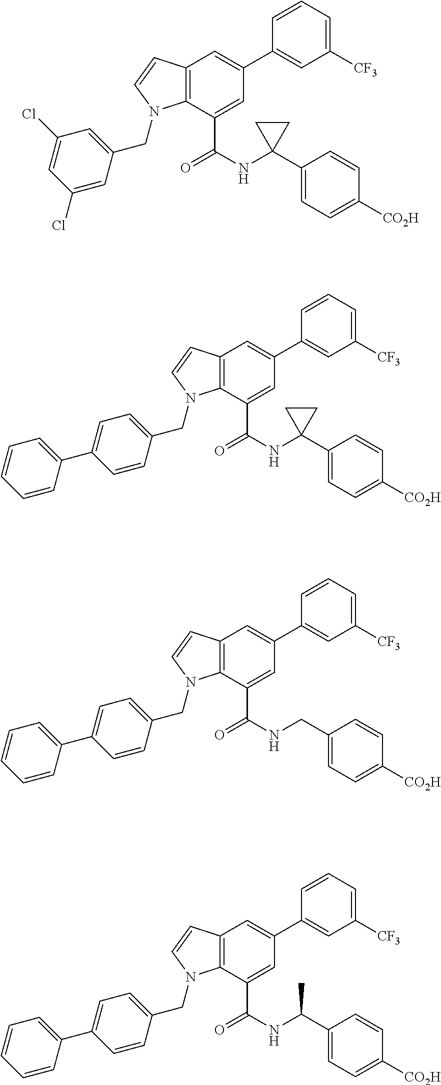 [SMILES: O=C(O)c1ccc(C2(NC(=O)c3cc(-c4cccc(C(F)(F)F)c4)cc4ccn(Cc5cc(Cl)cc(Cl)c5)c34)CC2)cc1 ; O=C(O)c1ccc(C2(NC(=O)c3cc(-c4cccc(C(F)(F)F)c4)cc4ccn(Cc5ccc(-c6ccccc6)cc5)c34)CC2)cc1 ; O=C(O)c1ccc(CNC(=O)c2cc(-c3cccc(C(F)(F)F)c3)cc3ccn(Cc4ccc(-c5ccccc5)cc4)c23)cc1 ; C[C@H](NC(=O)c1cc(-c2cccc(C(F)(F)F)c2)cc2ccn(Cc3ccc(-c4ccccc4)cc3)c12)c1ccc(C(=O)O)cc1]

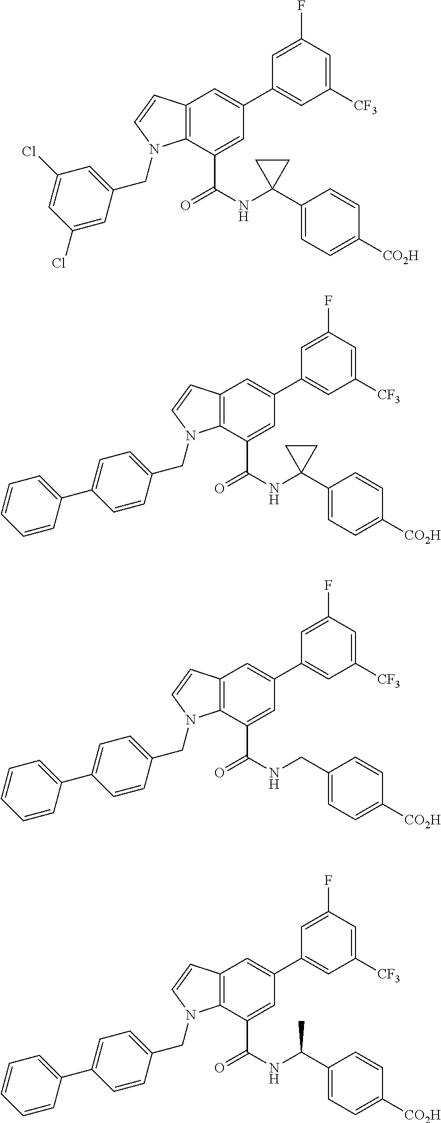 [SMILES: O=C(O)c1ccc(C2(NC(=O)c3cc(-c4cc(F)cc(C(F)(F)F)c4)cc4ccn(Cc5cc(Cl)cc(Cl)c5)c34)CC2)cc1 ; O=C(O)c1ccc(C2(NC(=O)c3cc(-c4cc(F)cc(C(F)(F)F)c4)cc4ccn(Cc5ccc(-c6ccccc6)cc5)c34)CC2)cc1 ; O=C(O)c1ccc(CNC(=O)c2cc(-c3cc(F)cc(C(F)(F)F)c3)cc3ccn(Cc4ccc(-c5ccccc5)cc4)c23)cc1 ; C[C@H](NC(=O)c1cc(-c2cc(F)cc(C(F)(F)F)c2)cc2ccn(Cc3ccc(-c4ccccc4)cc3)c12)c1ccc(C(=O)O)cc1]

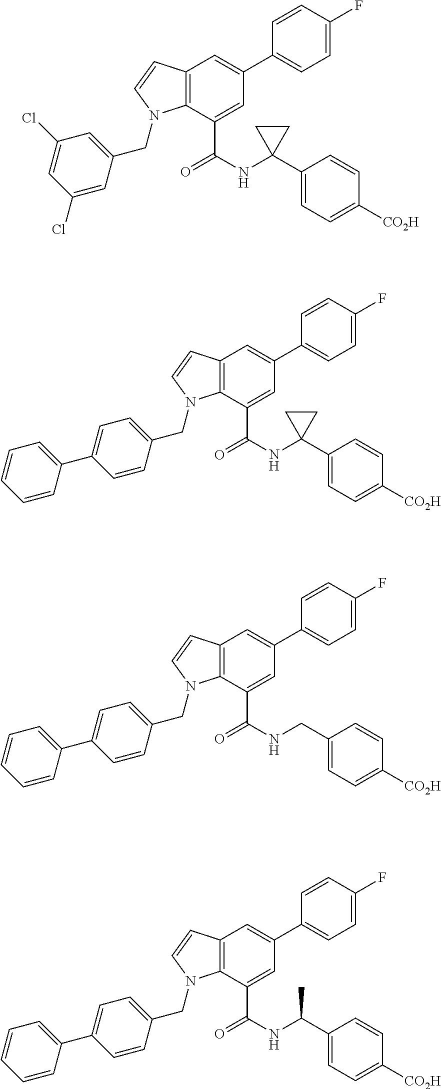 [SMILES: O=C(O)c1ccc(C2(NC(=O)c3cc(-c4ccc(F)cc4)cc4ccn(Cc5cc(Cl)cc(Cl)c5)c34)CC2)cc1 ; O=C(O)c1ccc(C2(NC(=O)c3cc(-c4ccc(F)cc4)cc4ccn(Cc5ccc(-c6ccccc6)cc5)c34)CC2)cc1 ; O=C(O)c1ccc(CNC(=O)c2cc(-c3ccc(F)cc3)cc3ccn(Cc4ccc(-c5ccccc5)cc4)c23)cc1 ; C[C@H](NC(=O)c1cc(-c2ccc(F)cc2)cc2ccn(Cc3ccc(-c4ccccc4)cc3)c12)c1ccc(C(=O)O)cc1]

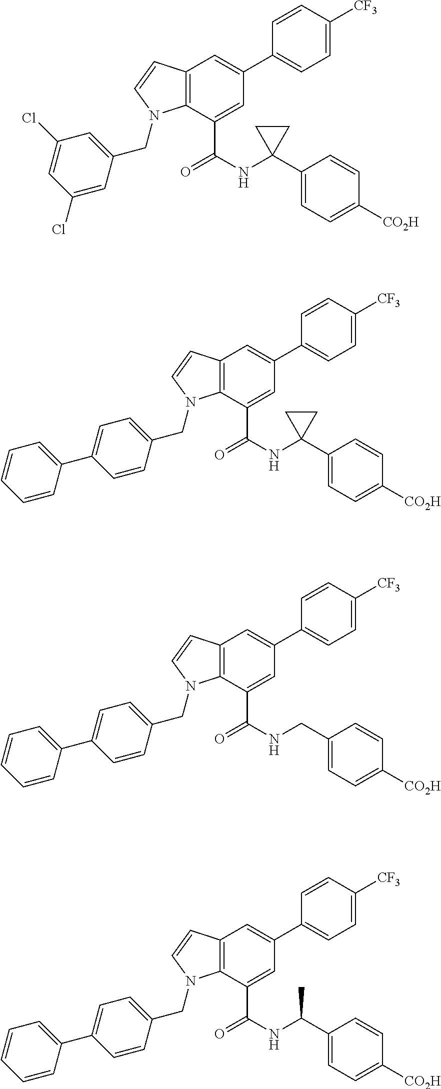 [SMILES: O=C(O)c1ccc(C2(NC(=O)c3cc(-c4ccc(C(F)(F)F)cc4)cc4ccn(Cc5cc(Cl)cc(Cl)c5)c34)CC2)cc1 ; O=C(O)c1ccc(C2(NC(=O)c3cc(-c4ccc(C(F)(F)F)cc4)cc4ccn(Cc5ccc(-c6ccccc6)cc5)c34)CC2)cc1 ; O=C(O)c1ccc(CNC(=O)c2cc(-c3ccc(C(F)(F)F)cc3)cc3ccn(Cc4ccc(-c5ccccc5)cc4)c23)cc1 ; C[C@H](NC(=O)c1cc(-c2ccc(C(F)(F)F)cc2)cc2ccn(Cc3ccc(-c4ccccc4)cc3)c12)c1ccc(C(=O)O)cc1]

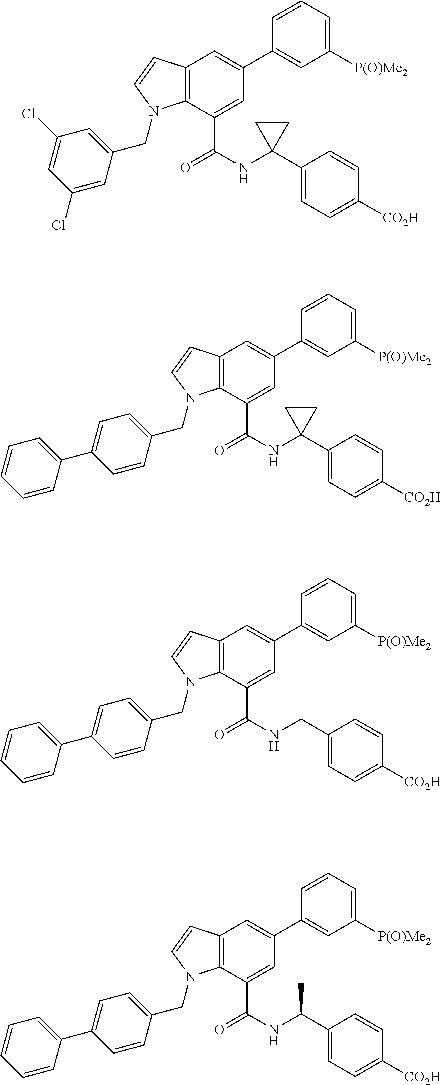

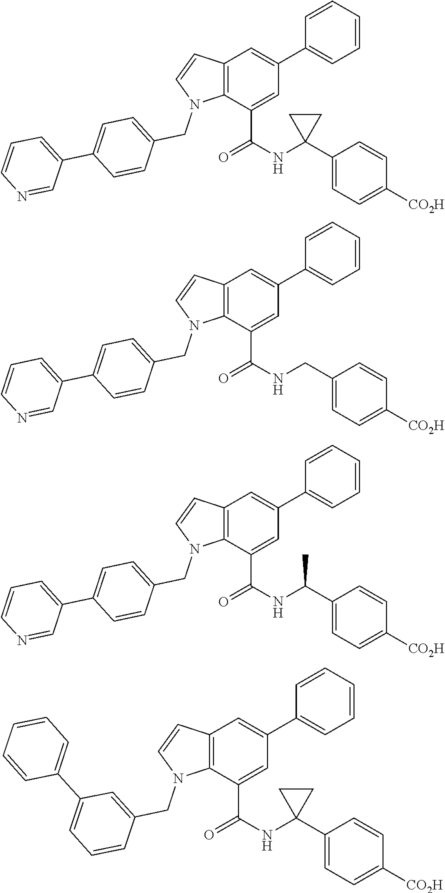

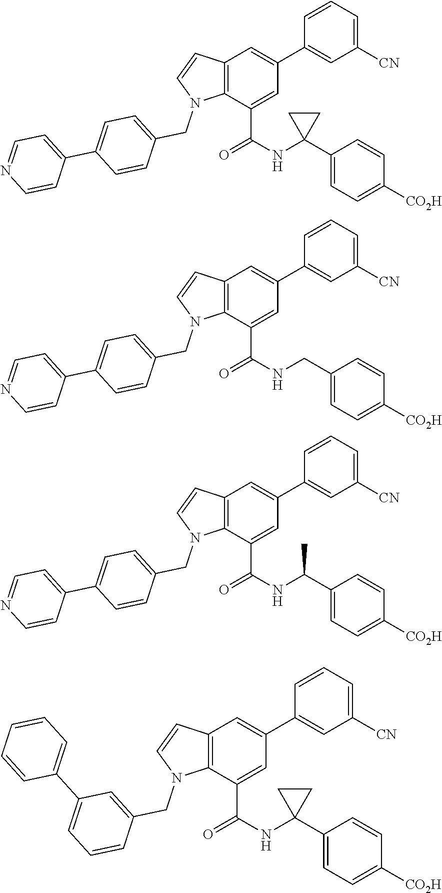

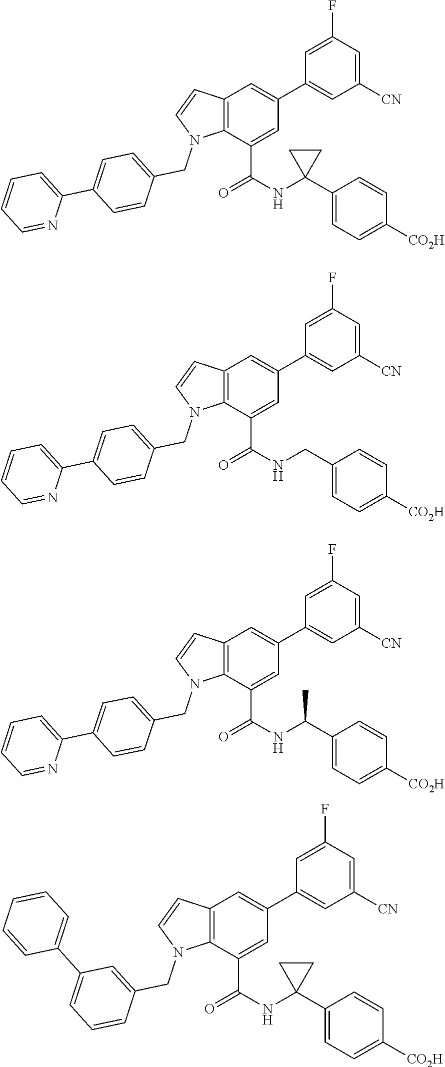

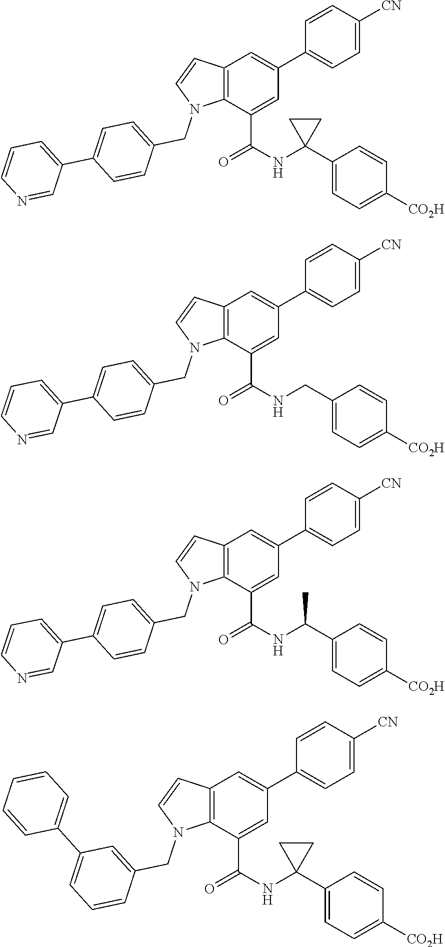

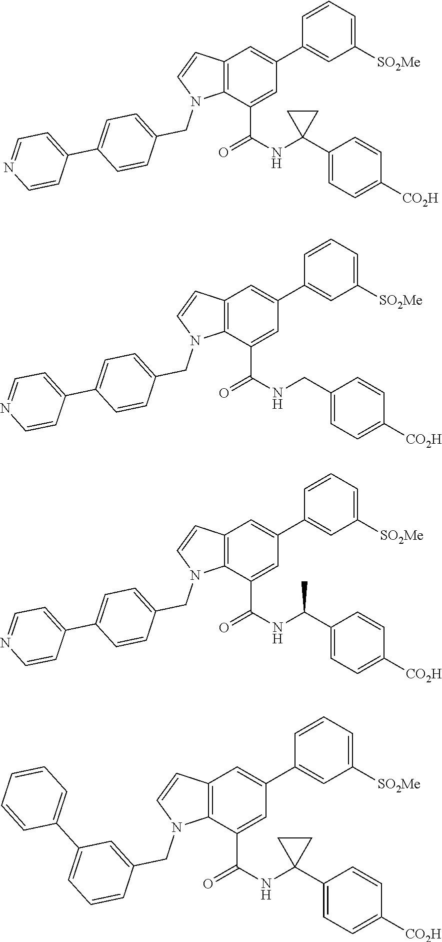 [SMILES: CS(=O)(=O)c1cccc(-c2cc(C(=O)NC3(c4ccc(C(=O)O)cc4)CC3)c3c(ccn3Cc3ccc(-c4ccncc4)cc3)c2)c1 ; CS(=O)(=O)c1cccc(-c2cc(C(=O)NCc3ccc(C(=O)O)cc3)c3c(ccn3Cc3ccc(-c4ccncc4)cc3)c2)c1 ; C[C@H](NC(=O)c1cc(-c2cccc(S(C)(=O)=O)c2)cc2ccn(Cc3ccc(-c4ccncc4)cc3)c12)c1ccc(C(=O)O)cc1 ; CS(=O)(=O)c1cccc(-c2cc(C(=O)NC3(c4ccc(C(=O)O)cc4)CC3)c3c(ccn3Cc3cccc(-c4ccccc4)c3)c2)c1]

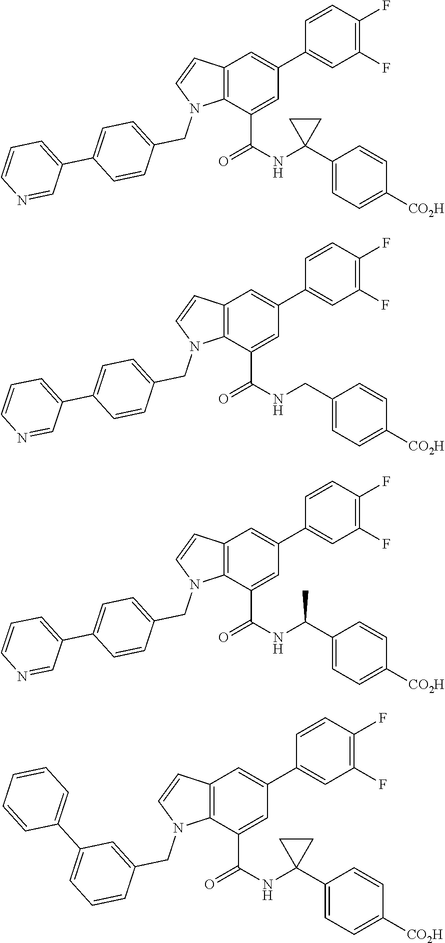

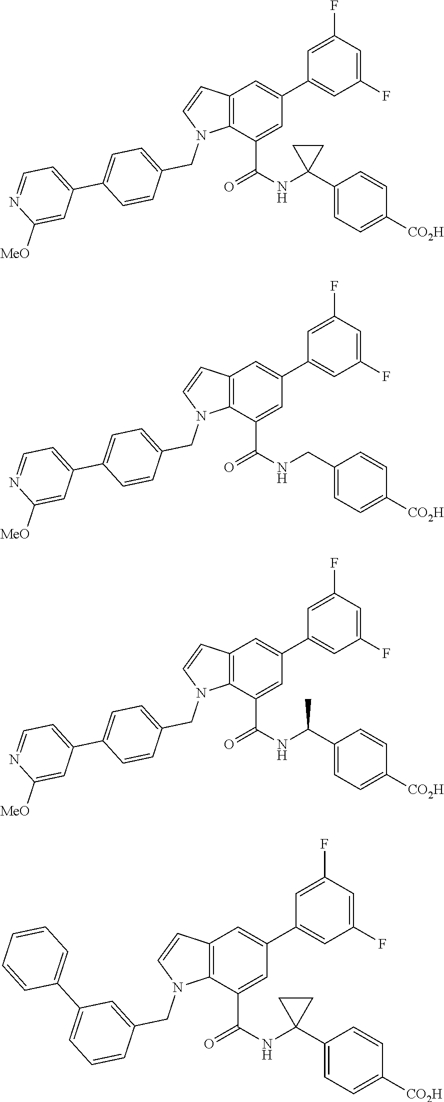 [SMILES: COc1cc(-c2ccc(Cn3ccc4cc(-c5cc(F)cc(F)c5)cc(C(=O)NC5(c6ccc(C(=O)O)cc6)CC5)c43)cc2)ccn1 ; COc1cc(-c2ccc(Cn3ccc4cc(-c5cc(F)cc(F)c5)cc(C(=O)NCc5ccc(C(=O)O)cc5)c43)cc2)ccn1 ; COc1cc(-c2ccc(Cn3ccc4cc(-c5cc(F)cc(F)c5)cc(C(=O)N[C@@H](C)c5ccc(C(=O)O)cc5)c43)cc2)ccn1 ; O=C(O)c1ccc(C2(NC(=O)c3cc(-c4cc(F)cc(F)c4)cc4ccn(Cc5cccc(-c6ccccc6)c5)c34)CC2)cc1]

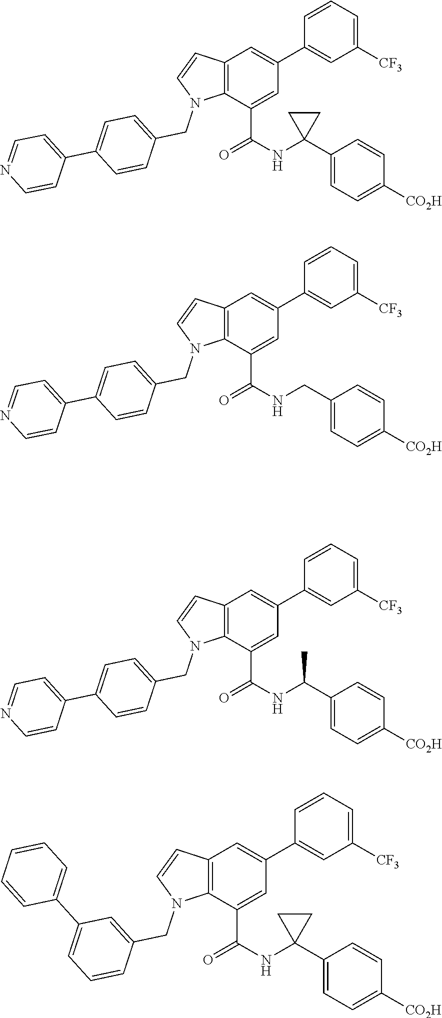 [SMILES: O=C(O)c1ccc(C2(NC(=O)c3cc(-c4cccc(C(F)(F)F)c4)cc4ccn(Cc5ccc(-c6ccncc6)cc5)c34)CC2)cc1 ; O=C(O)c1ccc(CNC(=O)c2cc(-c3cccc(C(F)(F)F)c3)cc3ccn(Cc4ccc(-c5ccncc5)cc4)c23)cc1 ; C[C@H](NC(=O)c1cc(-c2cccc(C(F)(F)F)c2)cc2ccn(Cc3ccc(-c4ccncc4)cc3)c12)c1ccc(C(=O)O)cc1 ; O=C(O)c1ccc(C2(NC(=O)c3cc(-c4cccc(C(F)(F)F)c4)cc4ccn(Cc5cccc(-c6ccccc6)c5)c34)CC2)cc1]

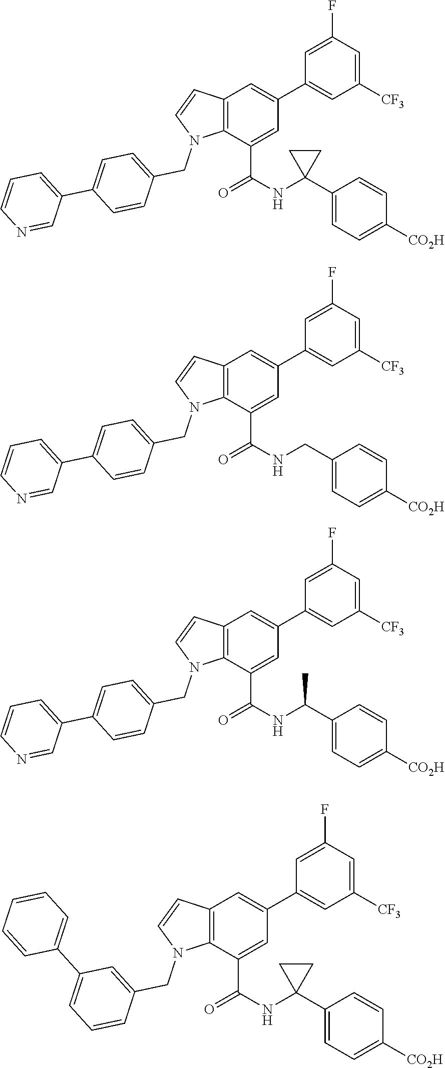

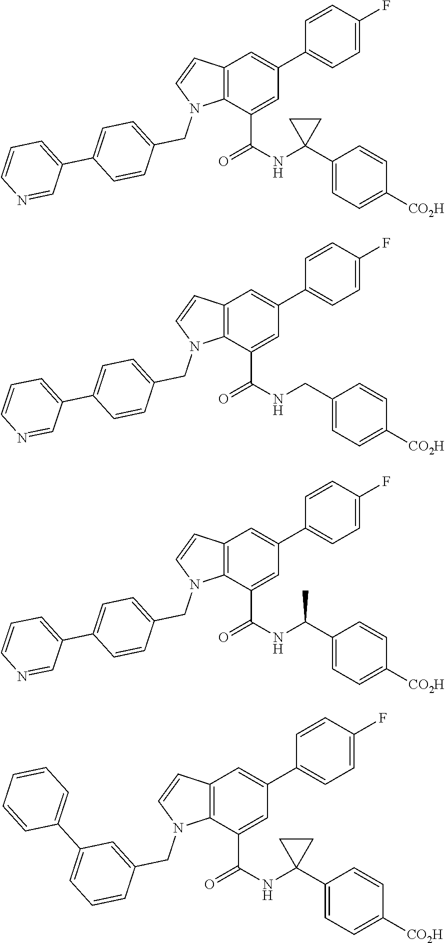

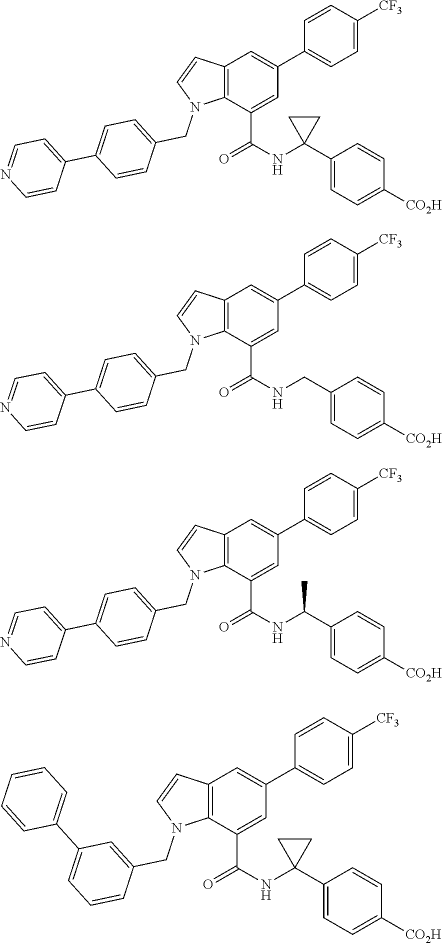 [SMILES: O=C(O)c1ccc(C2(NC(=O)c3cc(-c4ccc(C(F)(F)F)cc4)cc4ccn(Cc5ccc(-c6ccncc6)cc5)c34)CC2)cc1 ; O=C(O)c1ccc(CNC(=O)c2cc(-c3ccc(C(F)(F)F)cc3)cc3ccn(Cc4ccc(-c5ccncc5)cc4)c23)cc1 ; C[C@H](NC(=O)c1cc(-c2ccc(C(F)(F)F)cc2)cc2ccn(Cc3ccc(-c4ccncc4)cc3)c12)c1ccc(C(=O)O)cc1 ; O=C(O)c1ccc(C2(NC(=O)c3cc(-c4ccc(C(F)(F)F)cc4)cc4ccn(Cc5cccc(-c6ccccc6)c5)c34)CC2)cc1]

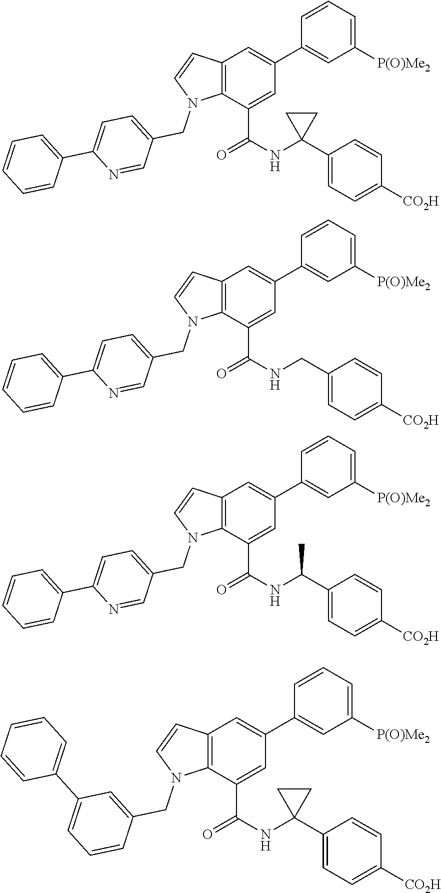

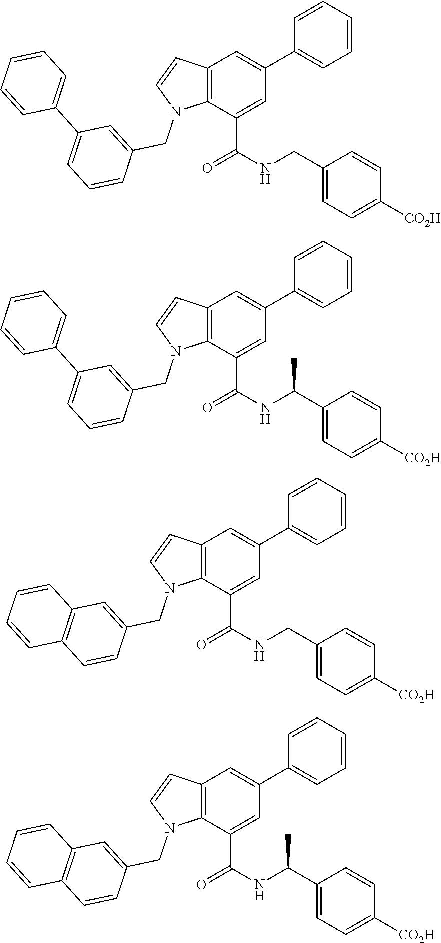

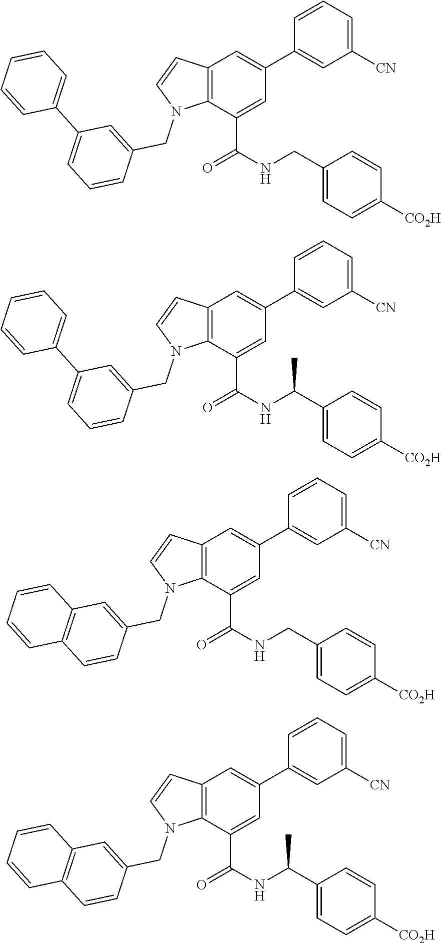

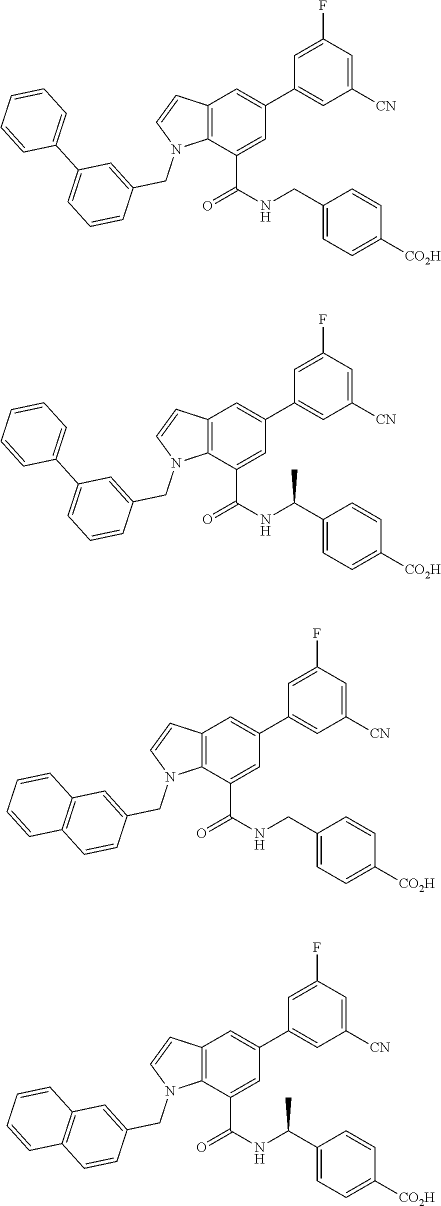 [SMILES: N#Cc1cc(F)cc(-c2cc(C(=O)NCc3ccc(C(=O)O)cc3)c3c(ccn3Cc3cccc(-c4ccccc4)c3)c2)c1 ; C[C@H](NC(=O)c1cc(-c2cc(F)cc(C#N)c2)cc2ccn(Cc3cccc(-c4ccccc4)c3)c12)c1ccc(C(=O)O)cc1 ; N#Cc1cc(F)cc(-c2cc(C(=O)NCc3ccc(C(=O)O)cc3)c3c(ccn3Cc3ccc4ccccc4c3)c2)c1 ; C[C@H](NC(=O)c1cc(-c2cc(F)cc(C#N)c2)cc2ccn(Cc3ccc4ccccc4c3)c12)c1ccc(C(=O)O)cc1]

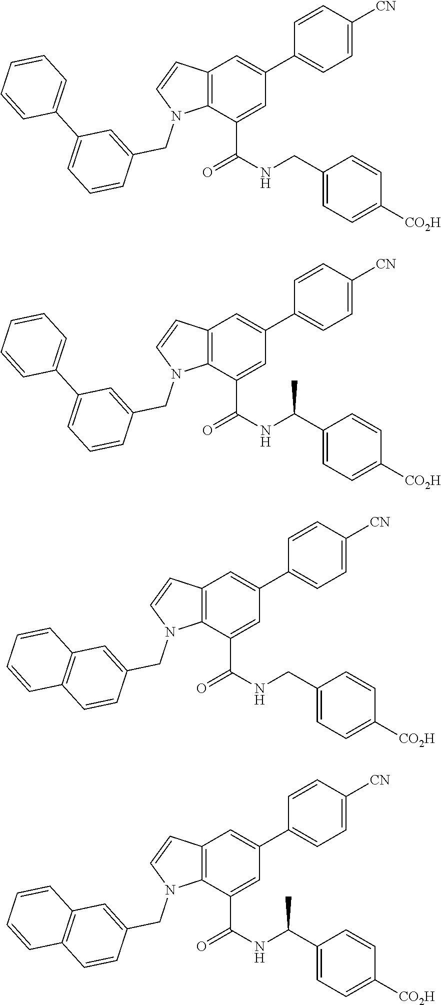 [SMILES: N#Cc1ccc(-c2cc(C(=O)NCc3ccc(C(=O)O)cc3)c3c(ccn3Cc3cccc(-c4ccccc4)c3)c2)cc1 ; C[C@H](NC(=O)c1cc(-c2ccc(C#N)cc2)cc2ccn(Cc3cccc(-c4ccccc4)c3)c12)c1ccc(C(=O)O)cc1 ; N#Cc1ccc(-c2cc(C(=O)NCc3ccc(C(=O)O)cc3)c3c(ccn3Cc3ccc4ccccc4c3)c2)cc1 ; C[C@H](NC(=O)c1cc(-c2ccc(C#N)cc2)cc2ccn(Cc3ccc4ccccc4c3)c12)c1ccc(C(=O)O)cc1]

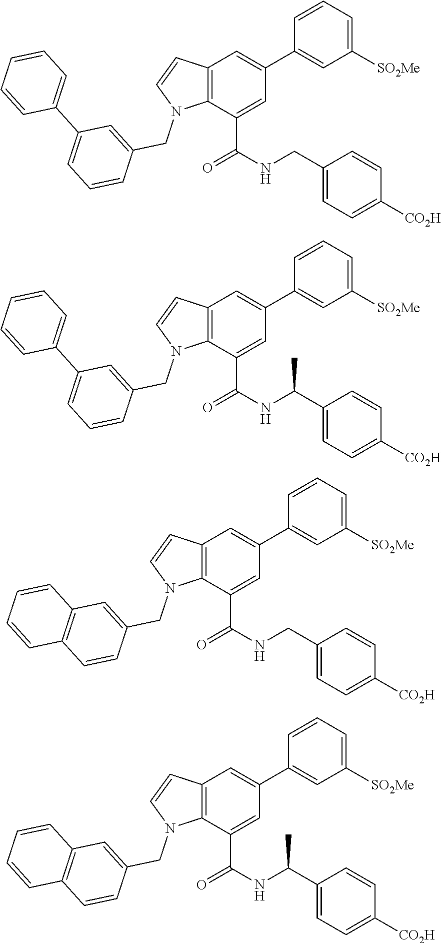

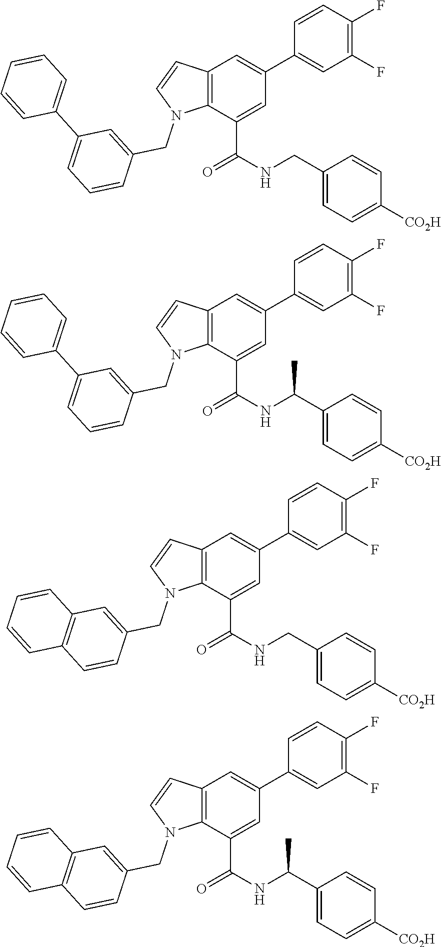

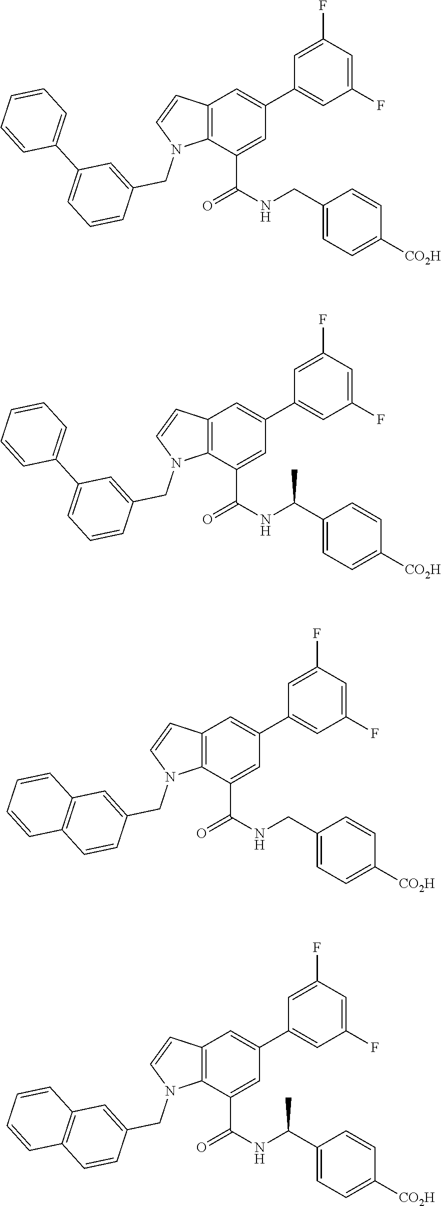 [SMILES: O=C(O)c1ccc(CNC(=O)c2cc(-c3cc(F)cc(F)c3)cc3ccn(Cc4cccc(-c5ccccc5)c4)c23)cc1 ; C[C@H](NC(=O)c1cc(-c2cc(F)cc(F)c2)cc2ccn(Cc3cccc(-c4ccccc4)c3)c12)c1ccc(C(=O)O)cc1 ; O=C(O)c1ccc(CNC(=O)c2cc(-c3cc(F)cc(F)c3)cc3ccn(Cc4ccc5ccccc5c4)c23)cc1 ; C[C@H](NC(=O)c1cc(-c2cc(F)cc(F)c2)cc2ccn(Cc3ccc4ccccc4c3)c12)c1ccc(C(=O)O)cc1]

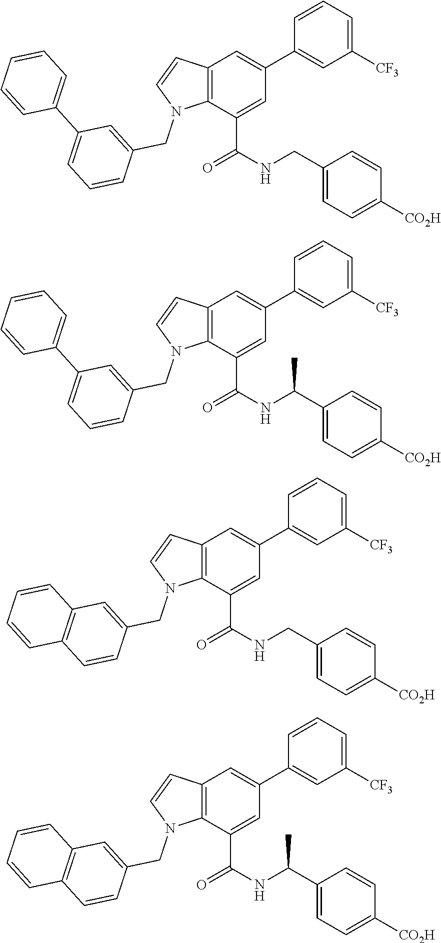

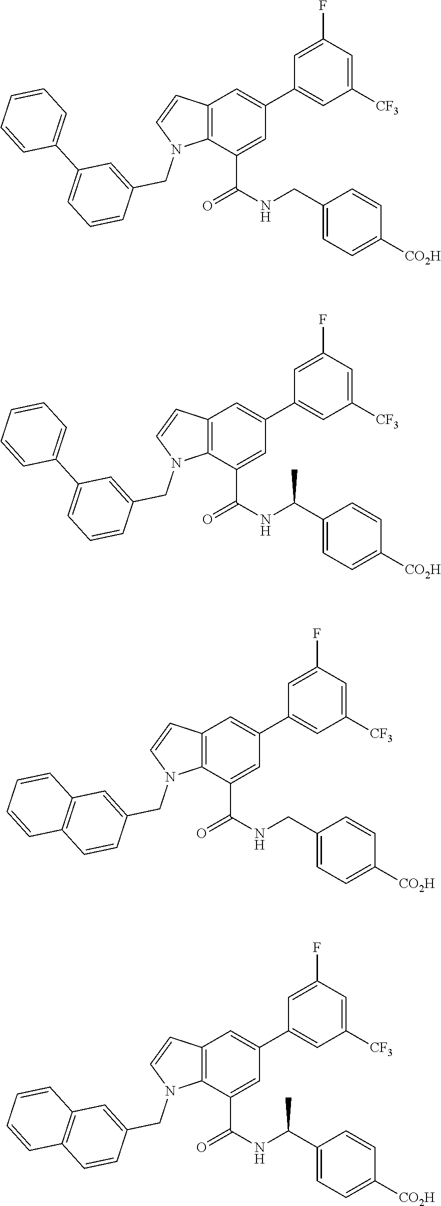 [SMILES: O=C(O)c1ccc(CNC(=O)c2cc(-c3cc(F)cc(C(F)(F)F)c3)cc3ccn(Cc4cccc(-c5ccccc5)c4)c23)cc1 ; C[C@H](NC(=O)c1cc(-c2cc(F)cc(C(F)(F)F)c2)cc2ccn(Cc3cccc(-c4ccccc4)c3)c12)c1ccc(C(=O)O)cc1 ; O=C(O)c1ccc(CNC(=O)c2cc(-c3cc(F)cc(C(F)(F)F)c3)cc3ccn(Cc4ccc5ccccc5c4)c23)cc1 ; C[C@H](NC(=O)c1cc(-c2cc(F)cc(C(F)(F)F)c2)cc2ccn(Cc3ccc4ccccc4c3)c12)c1ccc(C(=O)O)cc1]

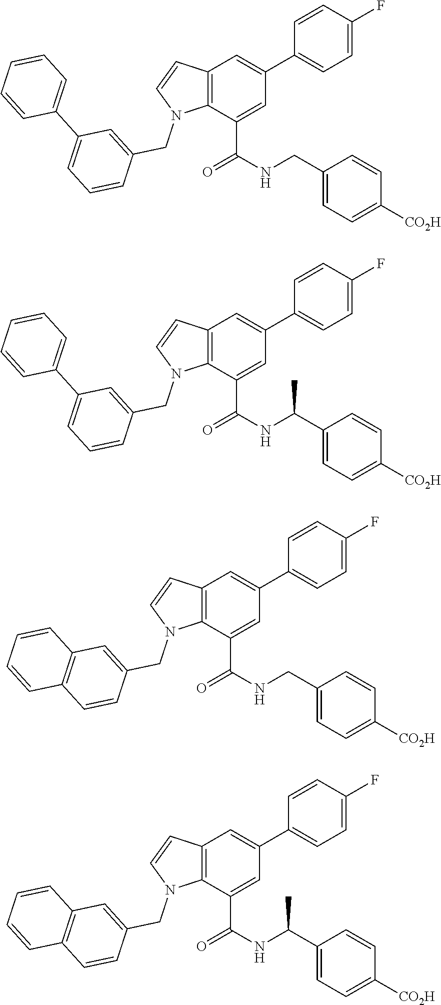 [SMILES: O=C(O)c1ccc(CNC(=O)c2cc(-c3ccc(F)cc3)cc3ccn(Cc4cccc(-c5ccccc5)c4)c23)cc1 ; C[C@H](NC(=O)c1cc(-c2ccc(F)cc2)cc2ccn(Cc3cccc(-c4ccccc4)c3)c12)c1ccc(C(=O)O)cc1 ; O=C(O)c1ccc(CNC(=O)c2cc(-c3ccc(F)cc3)cc3ccn(Cc4ccc5ccccc5c4)c23)cc1 ; C[C@H](NC(=O)c1cc(-c2ccc(F)cc2)cc2ccn(Cc3ccc4ccccc4c3)c12)c1ccc(C(=O)O)cc1]

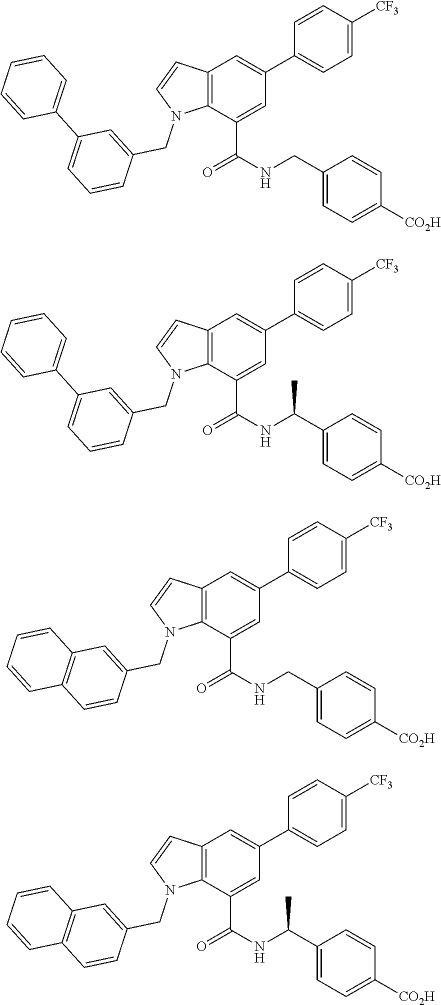 [SMILES: O=C(O)c1ccc(CNC(=O)c2cc(-c3ccc(C(F)(F)F)cc3)cc3ccn(Cc4cccc(-c5ccccc5)c4)c23)cc1 ; C[C@H](NC(=O)c1cc(-c2ccc(C(F)(F)F)cc2)cc2ccn(Cc3cccc(-c4ccccc4)c3)c12)c1ccc(C(=O)O)cc1 ; O=C(O)c1ccc(CNC(=O)c2cc(-c3ccc(C(F)(F)F)cc3)cc3ccn(Cc4ccc5ccccc5c4)c23)cc1 ; C[C@H](NC(=O)c1cc(-c2ccc(C(F)(F)F)cc2)cc2ccn(Cc3ccc4ccccc4c3)c12)c1ccc(C(=O)O)cc1]

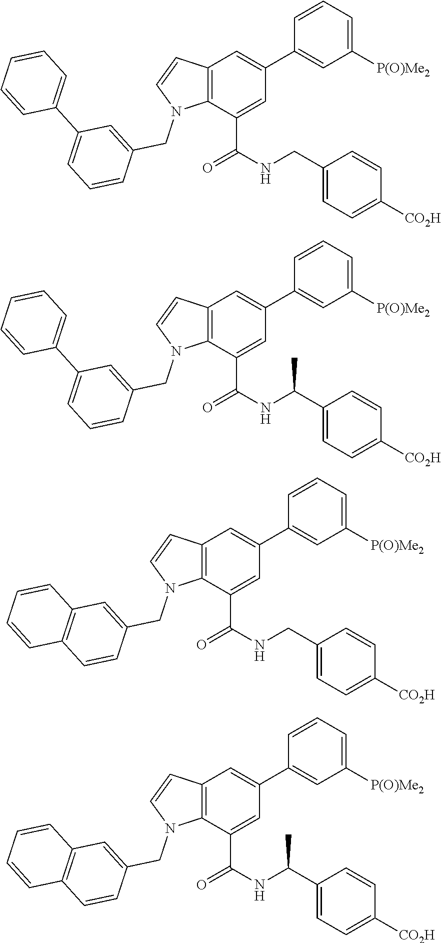

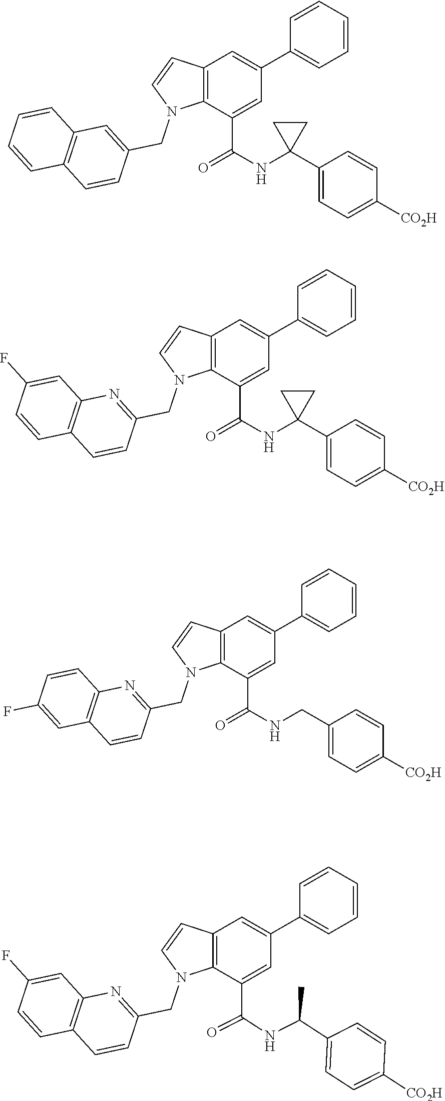 [SMILES: O=C(O)c1ccc(C2(NC(=O)c3cc(-c4ccccc4)cc4ccn(Cc5ccc6ccccc6c5)c34)CC2)cc1 ; O=C(O)c1ccc(C2(NC(=O)c3cc(-c4ccccc4)cc4ccn(Cc5ccc6ccc(F)cc6n5)c34)CC2)cc1 ; O=C(O)c1ccc(CNC(=O)c2cc(-c3ccccc3)cc3ccn(Cc4ccc5cc(F)ccc5n4)c23)cc1 ; C[C@H](NC(=O)c1cc(-c2ccccc2)cc2ccn(Cc3ccc4ccc(F)cc4n3)c12)c1ccc(C(=O)O)cc1]

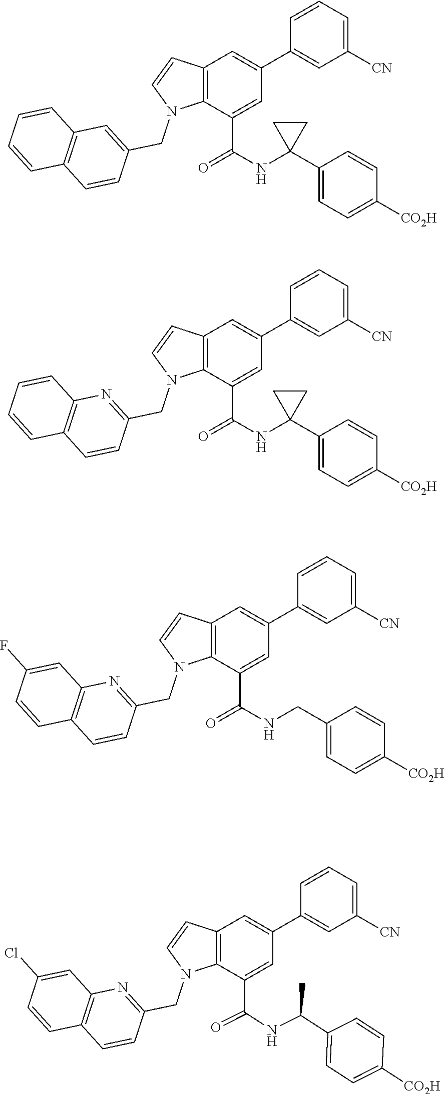 [SMILES: N#Cc1cccc(-c2cc(C(=O)NC3(c4ccc(C(=O)O)cc4)CC3)c3c(ccn3Cc3ccc4ccccc4c3)c2)c1 ; N#Cc1cccc(-c2cc(C(=O)NC3(c4ccc(C(=O)O)cc4)CC3)c3c(ccn3Cc3ccc4ccccc4n3)c2)c1 ; N#Cc1cccc(-c2cc(C(=O)NCc3ccc(C(=O)O)cc3)c3c(ccn3Cc3ccc4ccc(F)cc4n3)c2)c1 ; C[C@H](NC(=O)c1cc(-c2cccc(C#N)c2)cc2ccn(Cc3ccc4ccc(Cl)cc4n3)c12)c1ccc(C(=O)O)cc1]

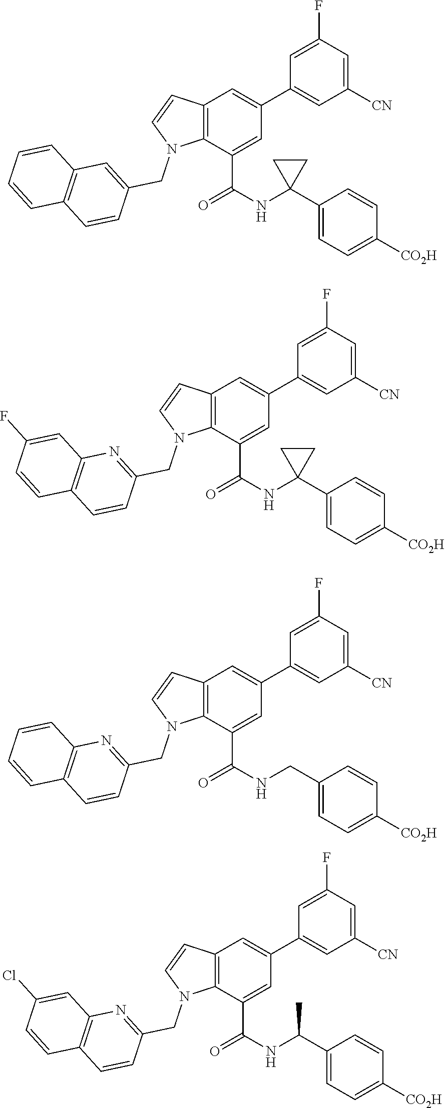

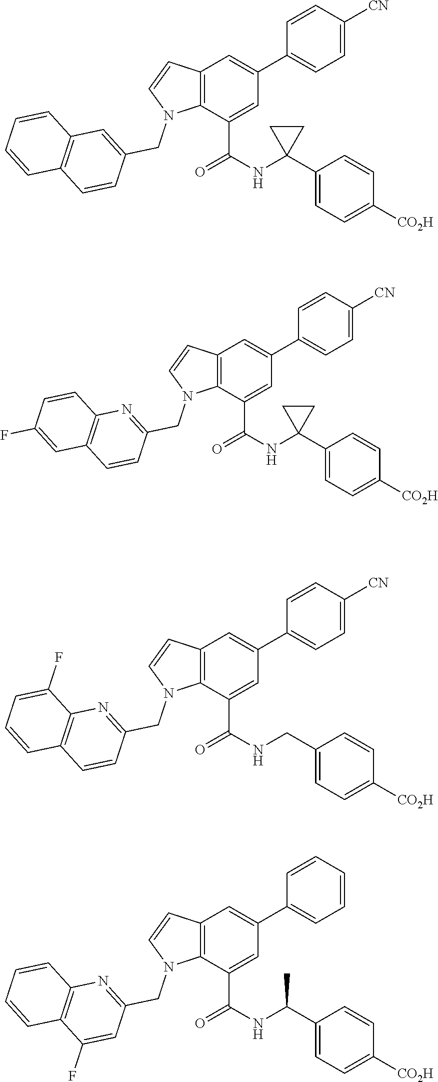 [SMILES: N#Cc1ccc(-c2cc(C(=O)NC3(c4ccc(C(=O)O)cc4)CC3)c3c(ccn3Cc3ccc4ccccc4c3)c2)cc1 ; N#Cc1ccc(-c2cc(C(=O)NC3(c4ccc(C(=O)O)cc4)CC3)c3c(ccn3Cc3ccc4cc(F)ccc4n3)c2)cc1 ; N#Cc1ccc(-c2cc(C(=O)NCc3ccc(C(=O)O)cc3)c3c(ccn3Cc3ccc4cccc(F)c4n3)c2)cc1 ; C[C@H](NC(=O)c1cc(-c2ccccc2)cc2ccn(Cc3cc(F)c4ccccc4n3)c12)c1ccc(C(=O)O)cc1]

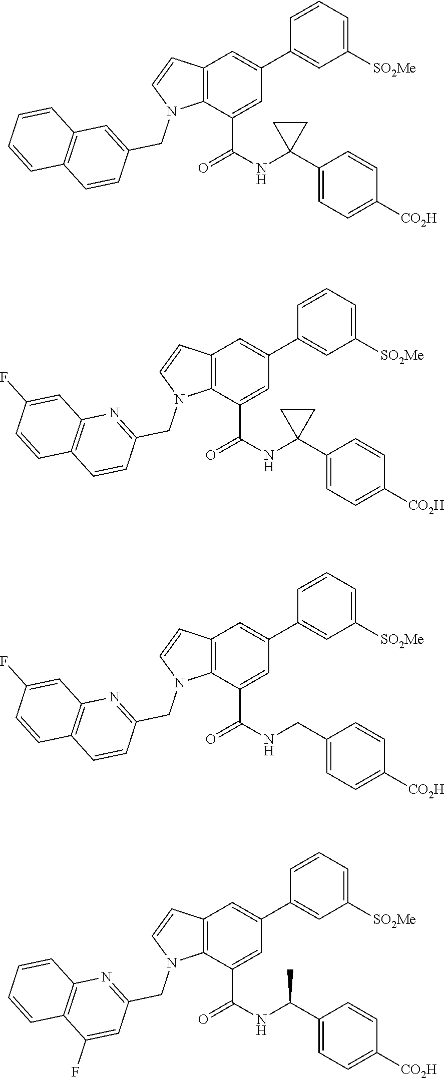 [SMILES: CS(=O)(=O)c1cccc(-c2cc(C(=O)NC3(c4ccc(C(=O)O)cc4)CC3)c3c(ccn3Cc3ccc4ccccc4c3)c2)c1 ; CS(=O)(=O)c1cccc(-c2cc(C(=O)NC3(c4ccc(C(=O)O)cc4)CC3)c3c(ccn3Cc3ccc4ccc(F)cc4n3)c2)c1 ; CS(=O)(=O)c1cccc(-c2cc(C(=O)NCc3ccc(C(=O)O)cc3)c3c(ccn3Cc3ccc4ccc(F)cc4n3)c2)c1 ; C[C@H](NC(=O)c1cc(-c2cccc(S(C)(=O)=O)c2)cc2ccn(Cc3cc(F)c4ccccc4n3)c12)c1ccc(C(=O)O)cc1]

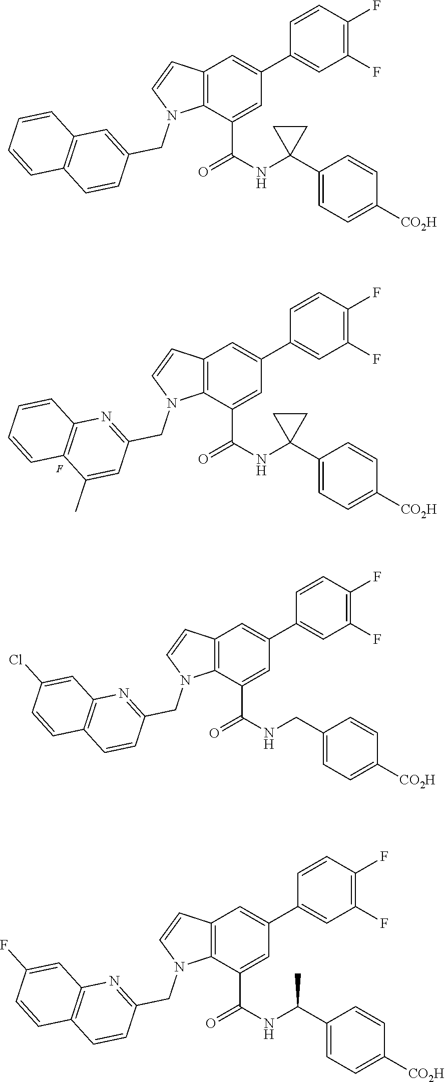

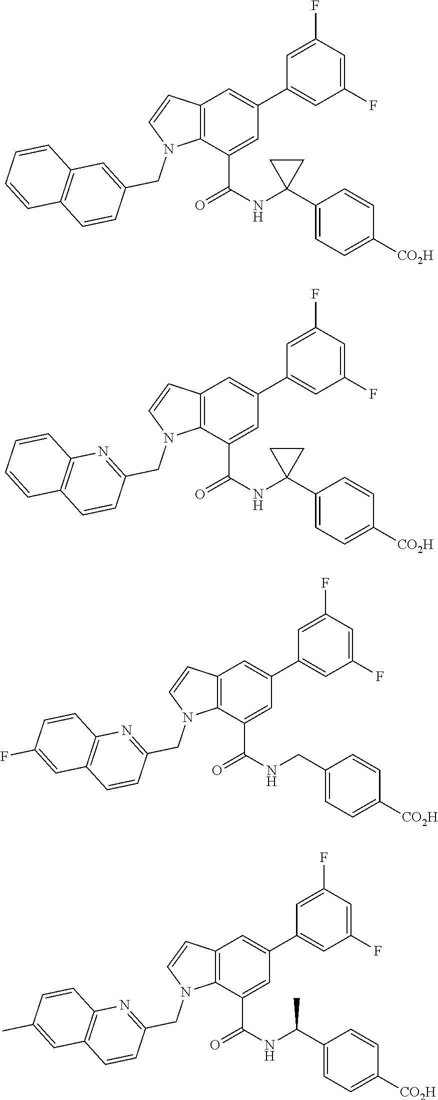

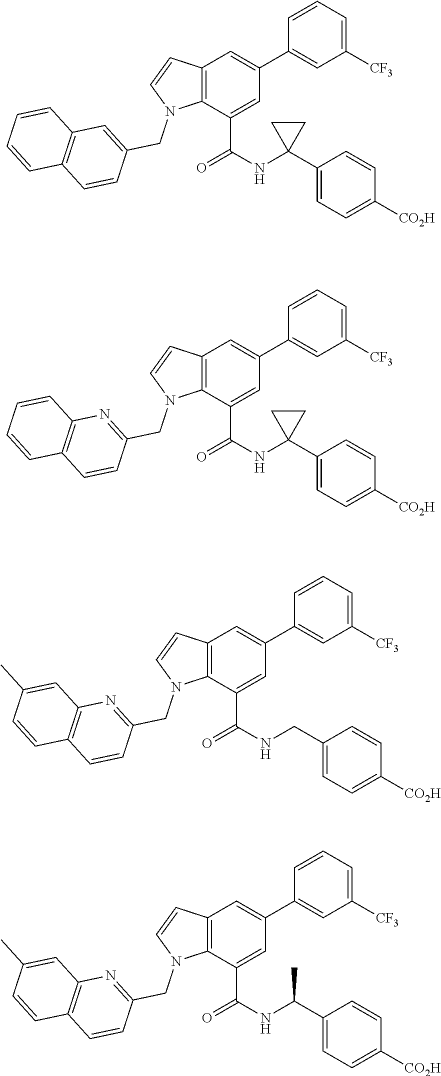 [SMILES: O=C(O)c1ccc(C2(NC(=O)c3cc(-c4cccc(C(F)(F)F)c4)cc4ccn(Cc5ccc6ccccc6c5)c34)CC2)cc1 ; O=C(O)c1ccc(C2(NC(=O)c3cc(-c4cccc(C(F)(F)F)c4)cc4ccn(Cc5ccc6ccccc6n5)c34)CC2)cc1 ; Cc1ccc2ccc(Cn3ccc4cc(-c5cccc(C(F)(F)F)c5)cc(C(=O)NCc5ccc(C(=O)O)cc5)c43)nc2c1 ; Cc1ccc2ccc(Cn3ccc4cc(-c5cccc(C(F)(F)F)c5)cc(C(=O)N[C@@H](C)c5ccc(C(=O)O)cc5)c43)nc2c1]

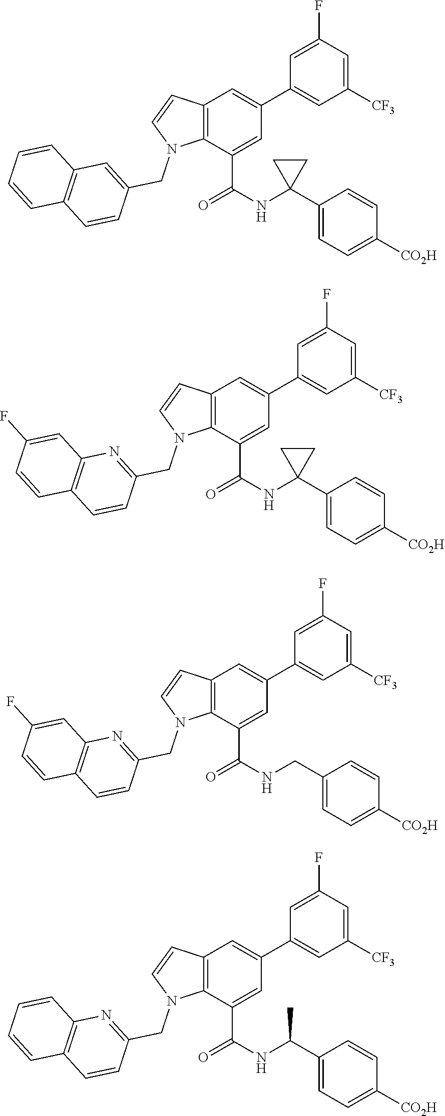

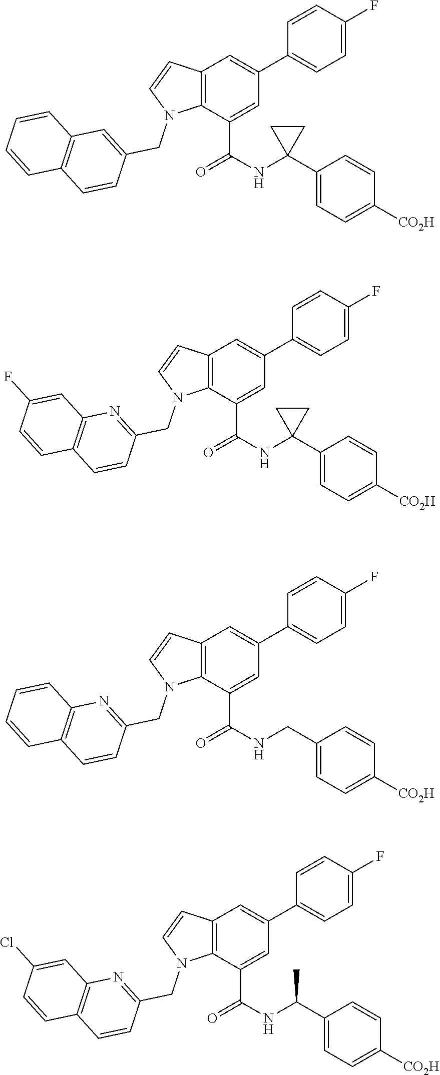 [SMILES: O=C(O)c1ccc(C2(NC(=O)c3cc(-c4ccc(F)cc4)cc4ccn(Cc5ccc6ccccc6c5)c34)CC2)cc1 ; O=C(O)c1ccc(C2(NC(=O)c3cc(-c4ccc(F)cc4)cc4ccn(Cc5ccc6ccc(F)cc6n5)c34)CC2)cc1 ; O=C(O)c1ccc(CNC(=O)c2cc(-c3ccc(F)cc3)cc3ccn(Cc4ccc5ccccc5n4)c23)cc1 ; C[C@H](NC(=O)c1cc(-c2ccc(F)cc2)cc2ccn(Cc3ccc4ccc(Cl)cc4n3)c12)c1ccc(C(=O)O)cc1]

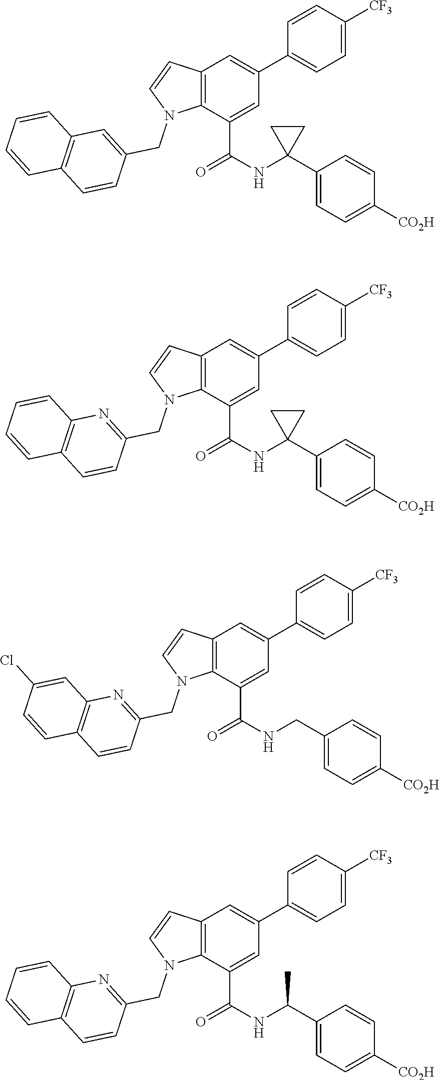 [SMILES: O=C(O)c1ccc(C2(NC(=O)c3cc(-c4ccc(C(F)(F)F)cc4)cc4ccn(Cc5ccc6ccccc6c5)c34)CC2)cc1 ; O=C(O)c1ccc(C2(NC(=O)c3cc(-c4ccc(C(F)(F)F)cc4)cc4ccn(Cc5ccc6ccccc6n5)c34)CC2)cc1 ; O=C(O)c1ccc(CNC(=O)c2cc(-c3ccc(C(F)(F)F)cc3)cc3ccn(Cc4ccc5ccc(Cl)cc5n4)c23)cc1 ; C[C@H](NC(=O)c1cc(-c2ccc(C(F)(F)F)cc2)cc2ccn(Cc3ccc4ccccc4n3)c12)c1ccc(C(=O)O)cc1]

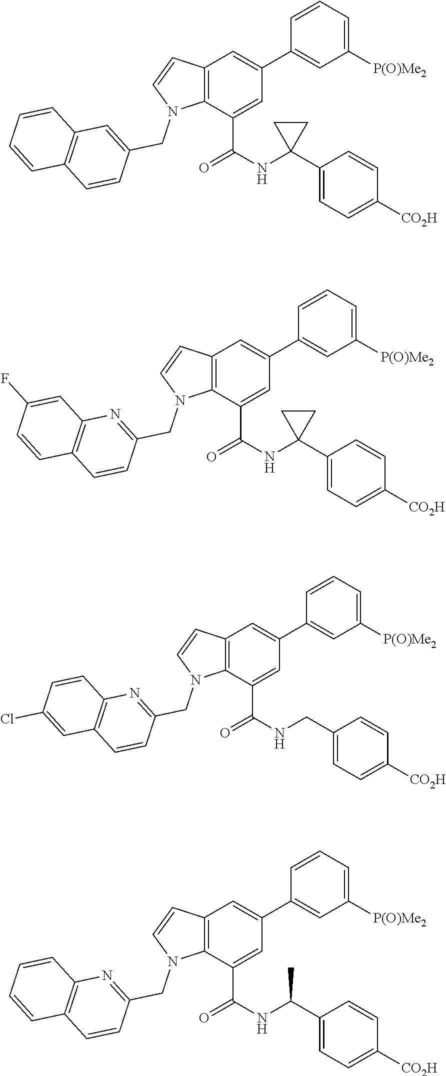

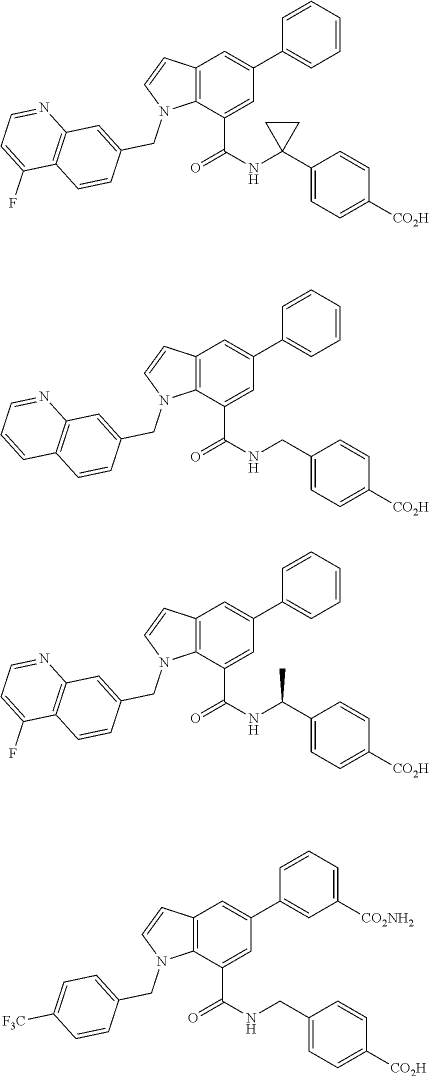

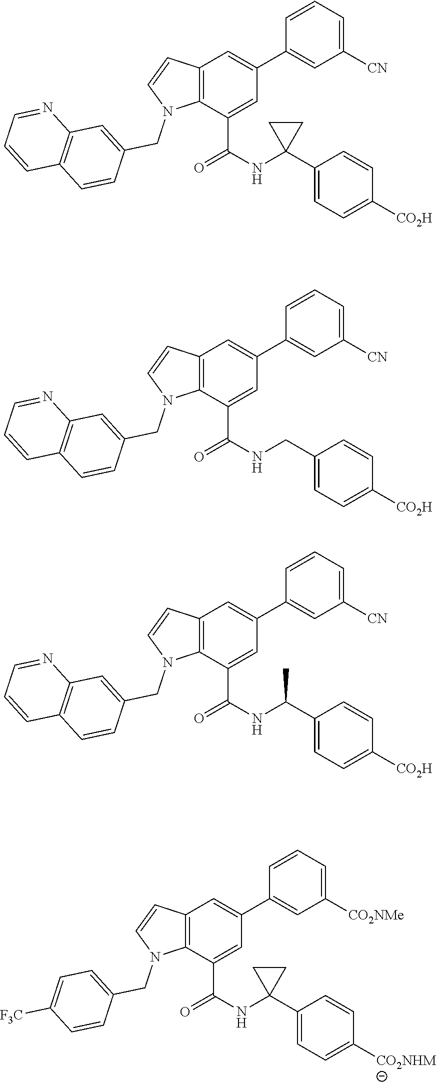

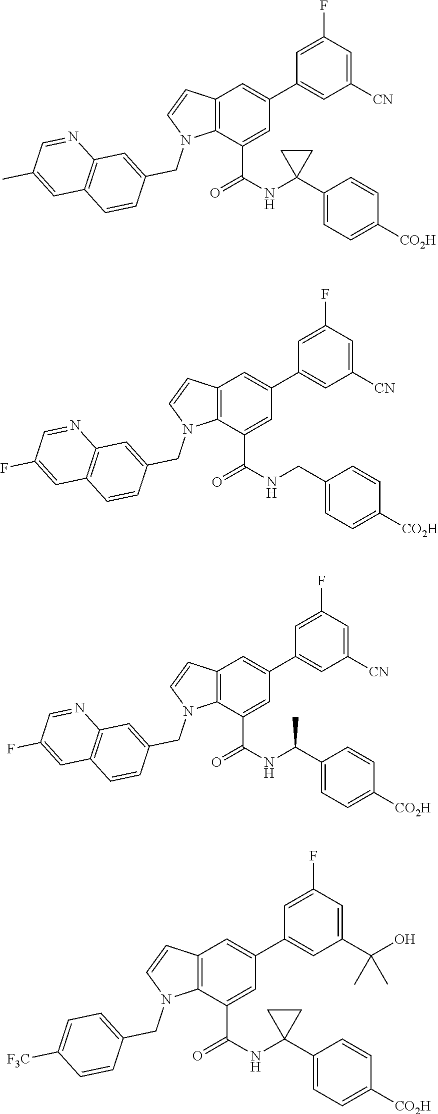 [SMILES: Cc1cnc2cc(Cn3ccc4cc(-c5cc(F)cc(C#N)c5)cc(C(=O)NC5(c6ccc(C(=O)O)cc6)CC5)c43)ccc2c1 ; N#Cc1cc(F)cc(-c2cc(C(=O)NCc3ccc(C(=O)O)cc3)c3c(ccn3Cc3ccc4cc(F)cnc4c3)c2)c1 ; C[C@H](NC(=O)c1cc(-c2cc(F)cc(C#N)c2)cc2ccn(Cc3ccc4cc(F)cnc4c3)c12)c1ccc(C(=O)O)cc1 ; CC(C)(O)c1cc(F)cc(-c2cc(C(=O)NC3(c4ccc(C(=O)O)cc4)CC3)c3c(ccn3Cc3ccc(C(F)(F)F)cc3)c2)c1]

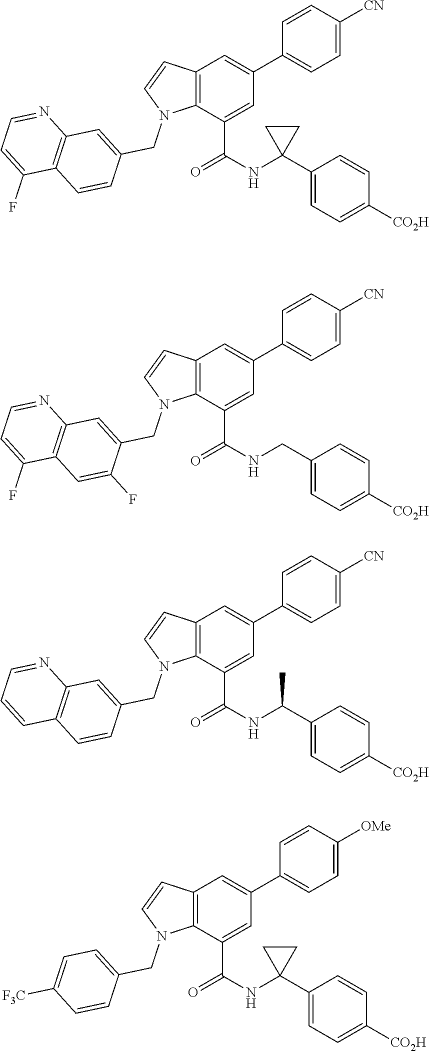 [SMILES: N#Cc1ccc(-c2cc(C(=O)NC3(c4ccc(C(=O)O)cc4)CC3)c3c(ccn3Cc3ccc4c(F)ccnc4c3)c2)cc1 ; N#Cc1ccc(-c2cc(C(=O)NCc3ccc(C(=O)O)cc3)c3c(ccn3Cc3cc4nccc(F)c4cc3F)c2)cc1 ; C[C@H](NC(=O)c1cc(-c2ccc(C#N)cc2)cc2ccn(Cc3ccc4cccnc4c3)c12)c1ccc(C(=O)O)cc1 ; COc1ccc(-c2cc(C(=O)NC3(c4ccc(C(=O)O)cc4)CC3)c3c(ccn3Cc3ccc(C(F)(F)F)cc3)c2)cc1]

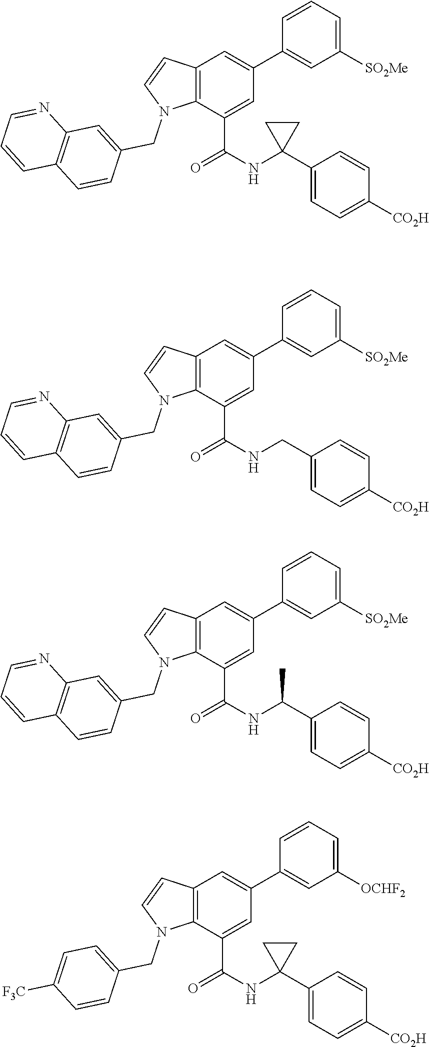 [SMILES: CS(=O)(=O)c1cccc(-c2cc(C(=O)NC3(c4ccc(C(=O)O)cc4)CC3)c3c(ccn3Cc3ccc4cccnc4c3)c2)c1 ; CS(=O)(=O)c1cccc(-c2cc(C(=O)NCc3ccc(C(=O)O)cc3)c3c(ccn3Cc3ccc4cccnc4c3)c2)c1 ; C[C@H](NC(=O)c1cc(-c2cccc(S(C)(=O)=O)c2)cc2ccn(Cc3ccc4cccnc4c3)c12)c1ccc(C(=O)O)cc1 ; O=C(O)c1ccc(C2(NC(=O)c3cc(-c4cccc(OC(F)F)c4)cc4ccn(Cc5ccc(C(F)(F)F)cc5)c34)CC2)cc1]

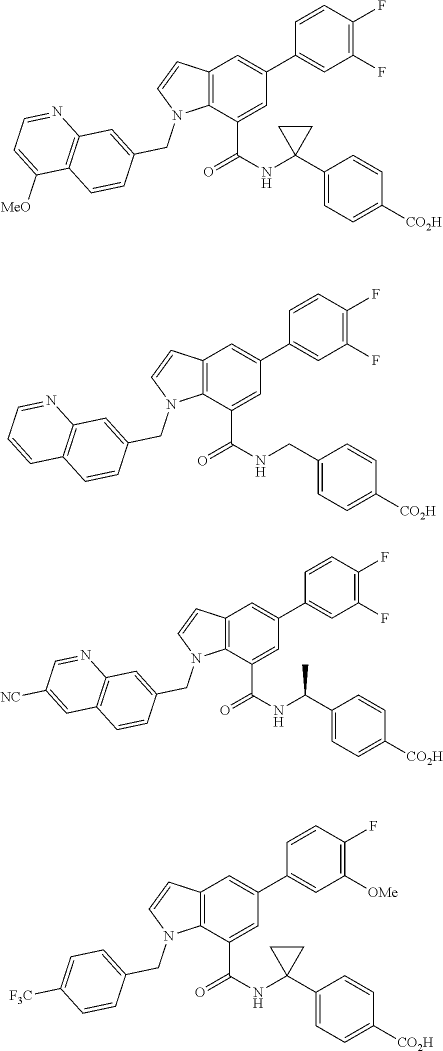 [SMILES: COc1ccnc2cc(Cn3ccc4cc(-c5ccc(F)c(F)c5)cc(C(=O)NC5(c6ccc(C(=O)O)cc6)CC5)c43)ccc12 ; O=C(O)c1ccc(CNC(=O)c2cc(-c3ccc(F)c(F)c3)cc3ccn(Cc4ccc5cccnc5c4)c23)cc1 ; C[C@H](NC(=O)c1cc(-c2ccc(F)c(F)c2)cc2ccn(Cc3ccc4cc(C#N)cnc4c3)c12)c1ccc(C(=O)O)cc1 ; COc1cc(-c2cc(C(=O)NC3(c4ccc(C(=O)O)cc4)CC3)c3c(ccn3Cc3ccc(C(F)(F)F)cc3)c2)ccc1F]

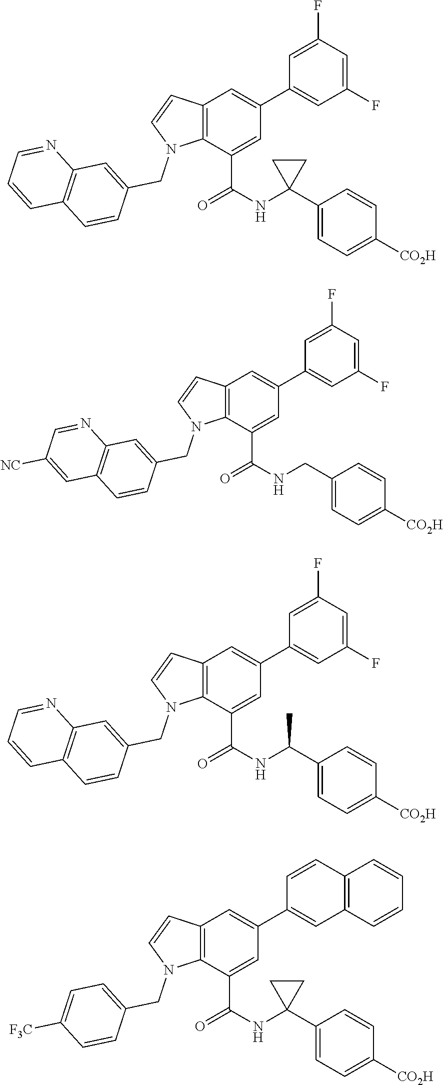

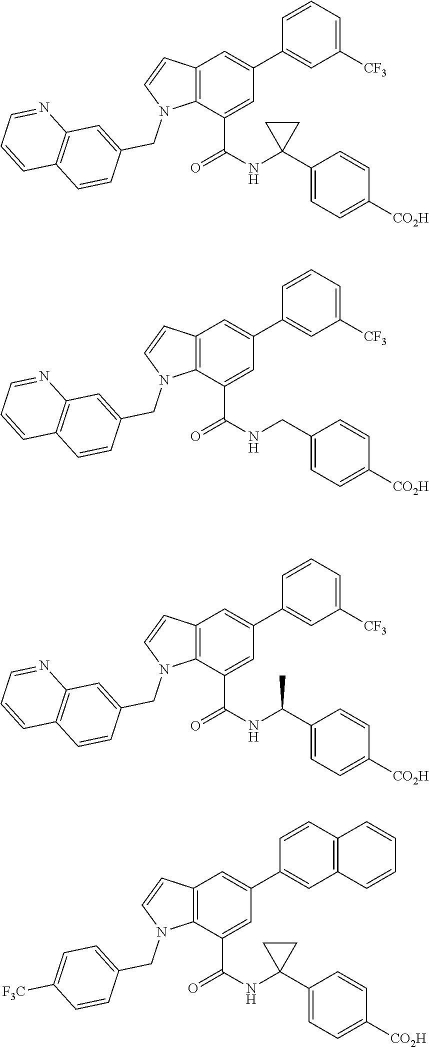 [SMILES: O=C(O)c1ccc(C2(NC(=O)c3cc(-c4cccc(C(F)(F)F)c4)cc4ccn(Cc5ccc6cccnc6c5)c34)CC2)cc1 ; O=C(O)c1ccc(CNC(=O)c2cc(-c3cccc(C(F)(F)F)c3)cc3ccn(Cc4ccc5cccnc5c4)c23)cc1 ; C[C@H](NC(=O)c1cc(-c2cccc(C(F)(F)F)c2)cc2ccn(Cc3ccc4cccnc4c3)c12)c1ccc(C(=O)O)cc1 ; O=C(O)c1ccc(C2(NC(=O)c3cc(-c4ccc5ccccc5c4)cc4ccn(Cc5ccc(C(F)(F)F)cc5)c34)CC2)cc1]

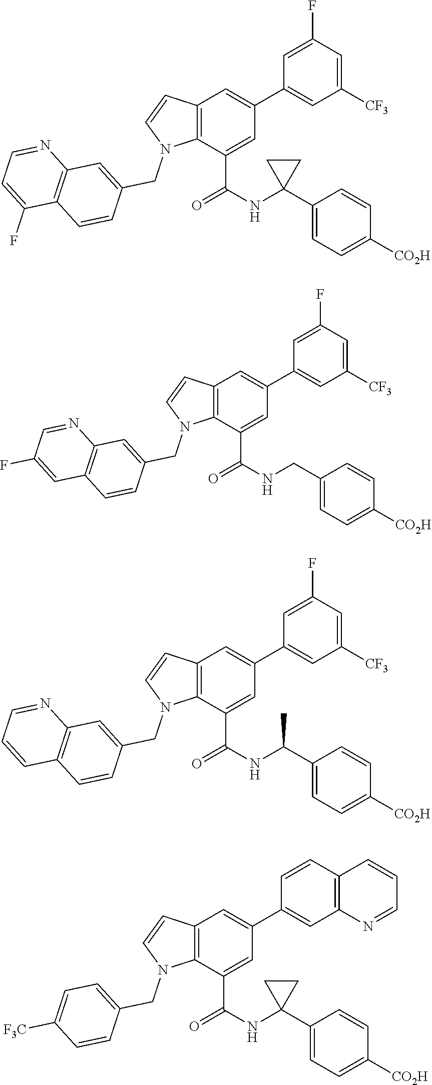

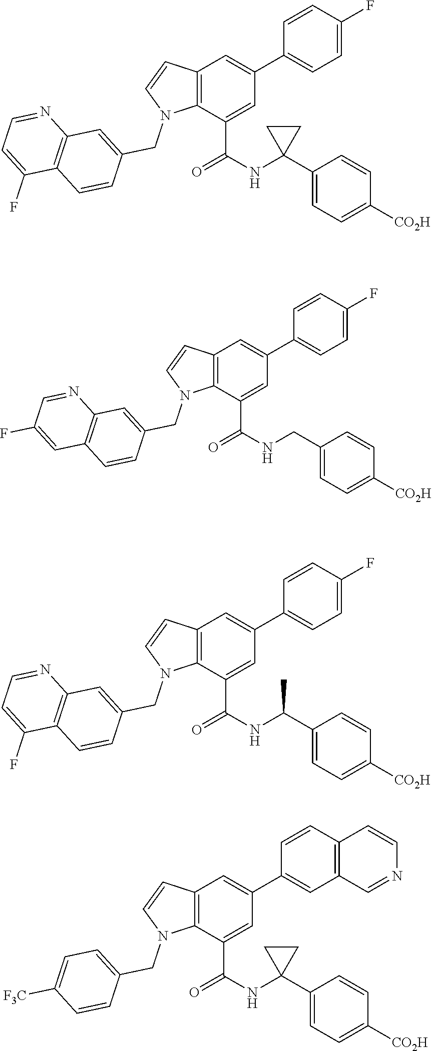 [SMILES: O=C(O)c1ccc(C2(NC(=O)c3cc(-c4ccc(F)cc4)cc4ccn(Cc5ccc6c(F)ccnc6c5)c34)CC2)cc1 ; O=C(O)c1ccc(CNC(=O)c2cc(-c3ccc(F)cc3)cc3ccn(Cc4ccc5cc(F)cnc5c4)c23)cc1 ; C[C@H](NC(=O)c1cc(-c2ccc(F)cc2)cc2ccn(Cc3ccc4c(F)ccnc4c3)c12)c1ccc(C(=O)O)cc1 ; O=C(O)c1ccc(C2(NC(=O)c3cc(-c4ccc5ccncc5c4)cc4ccn(Cc5ccc(C(F)(F)F)cc5)c34)CC2)cc1]

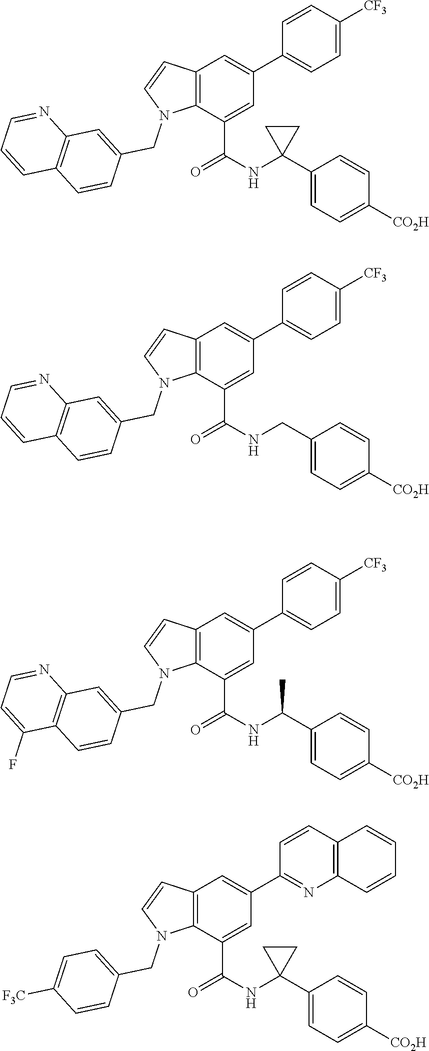 [SMILES: O=C(O)c1ccc(C2(NC(=O)c3cc(-c4ccc(C(F)(F)F)cc4)cc4ccn(Cc5ccc6cccnc6c5)c34)CC2)cc1 ; O=C(O)c1ccc(CNC(=O)c2cc(-c3ccc(C(F)(F)F)cc3)cc3ccn(Cc4ccc5cccnc5c4)c23)cc1 ; C[C@H](NC(=O)c1cc(-c2ccc(C(F)(F)F)cc2)cc2ccn(Cc3ccc4c(F)ccnc4c3)c12)c1ccc(C(=O)O)cc1 ; O=C(O)c1ccc(C2(NC(=O)c3cc(-c4ccc5ccccc5n4)cc4ccn(Cc5ccc(C(F)(F)F)cc5)c34)CC2)cc1]

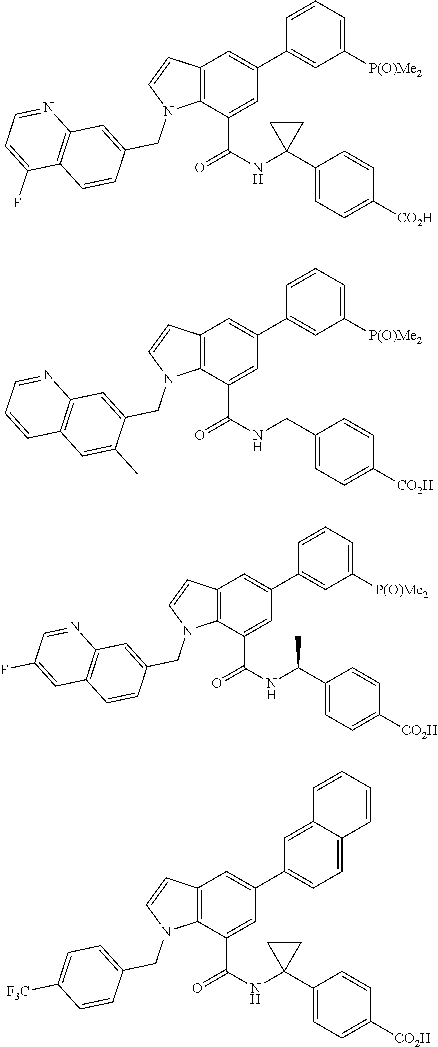

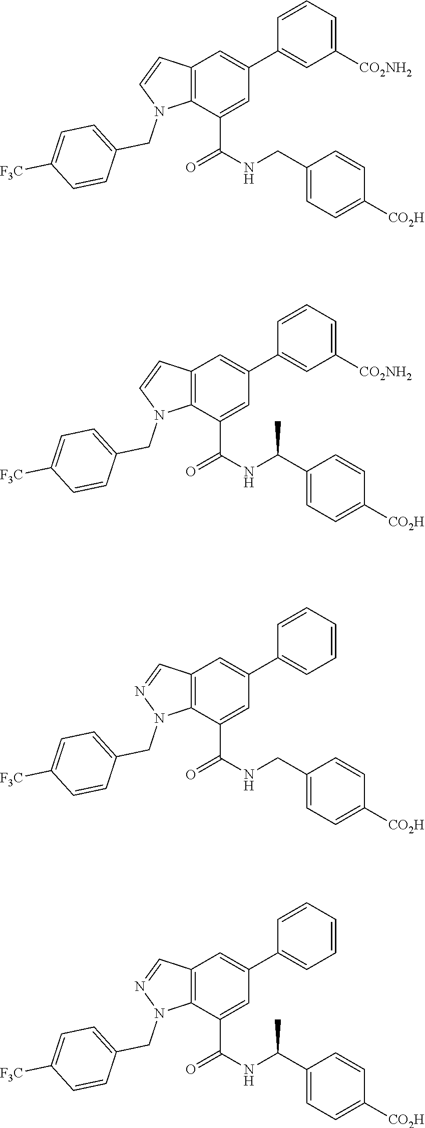

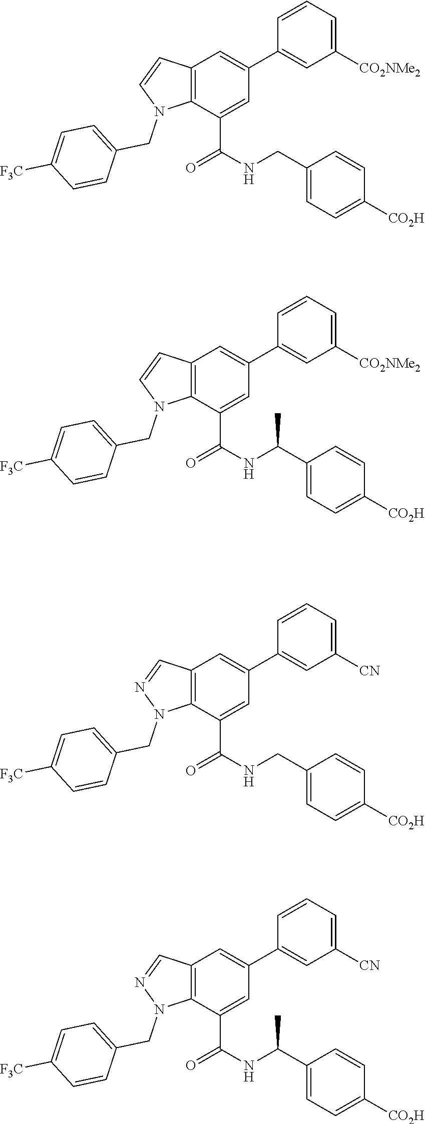

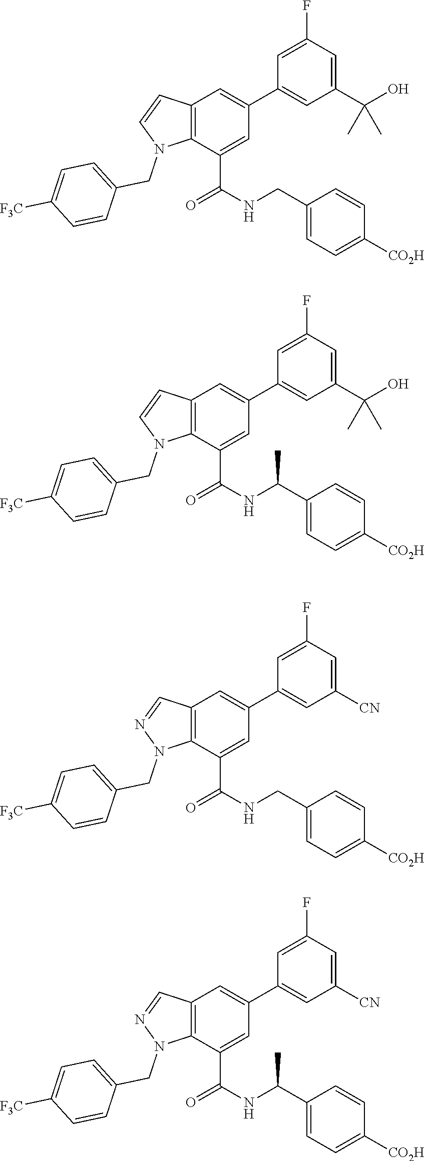 [SMILES: CC(C)(O)c1cc(F)cc(-c2cc(C(=O)NCc3ccc(C(=O)O)cc3)c3c(ccn3Cc3ccc(C(F)(F)F)cc3)c2)c1 ; C[C@H](NC(=O)c1cc(-c2cc(F)cc(C(C)(C)O)c2)cc2ccn(Cc3ccc(C(F)(F)F)cc3)c12)c1ccc(C(=O)O)cc1 ; N#Cc1cc(F)cc(-c2cc(C(=O)NCc3ccc(C(=O)O)cc3)c3c(cnn3Cc3ccc(C(F)(F)F)cc3)c2)c1 ; C[C@H](NC(=O)c1cc(-c2cc(F)cc(C#N)c2)cc2cnn(Cc3ccc(C(F)(F)F)cc3)c12)c1ccc(C(=O)O)cc1]

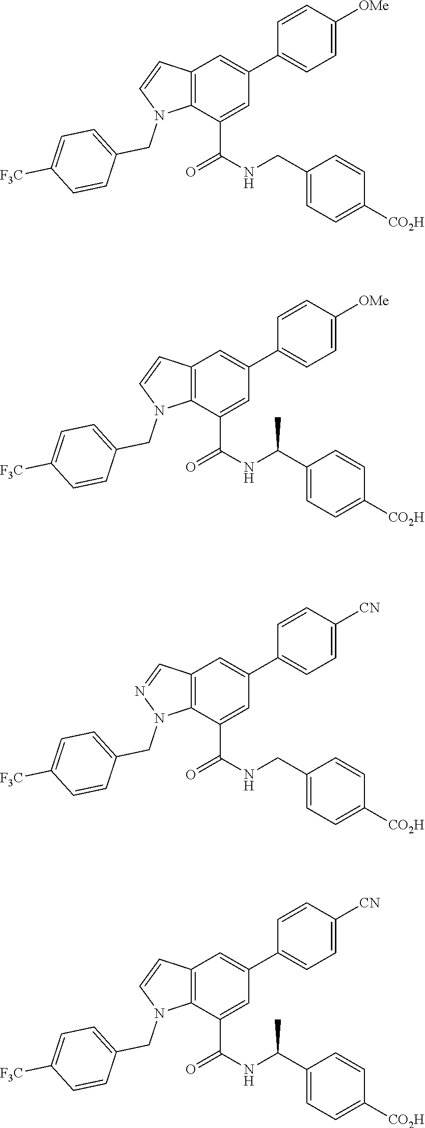 [SMILES: COc1ccc(-c2cc(C(=O)NCc3ccc(C(=O)O)cc3)c3c(ccn3Cc3ccc(C(F)(F)F)cc3)c2)cc1 ; COc1ccc(-c2cc(C(=O)N[C@@H](C)c3ccc(C(=O)O)cc3)c3c(ccn3Cc3ccc(C(F)(F)F)cc3)c2)cc1 ; N#Cc1ccc(-c2cc(C(=O)NCc3ccc(C(=O)O)cc3)c3c(cnn3Cc3ccc(C(F)(F)F)cc3)c2)cc1 ; C[C@H](NC(=O)c1cc(-c2ccc(C#N)cc2)cc2ccn(Cc3ccc(C(F)(F)F)cc3)c12)c1ccc(C(=O)O)cc1]

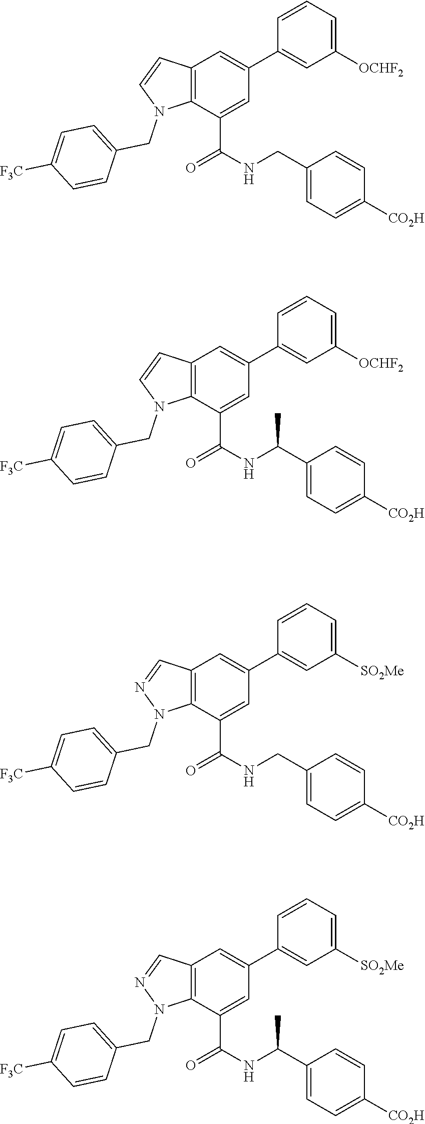 [SMILES: O=C(O)c1ccc(CNC(=O)c2cc(-c3cccc(OC(F)F)c3)cc3ccn(Cc4ccc(C(F)(F)F)cc4)c23)cc1 ; C[C@H](NC(=O)c1cc(-c2cccc(OC(F)F)c2)cc2ccn(Cc3ccc(C(F)(F)F)cc3)c12)c1ccc(C(=O)O)cc1 ; CS(=O)(=O)c1cccc(-c2cc(C(=O)NCc3ccc(C(=O)O)cc3)c3c(cnn3Cc3ccc(C(F)(F)F)cc3)c2)c1 ; C[C@H](NC(=O)c1cc(-c2cccc(S(C)(=O)=O)c2)cc2cnn(Cc3ccc(C(F)(F)F)cc3)c12)c1ccc(C(=O)O)cc1]

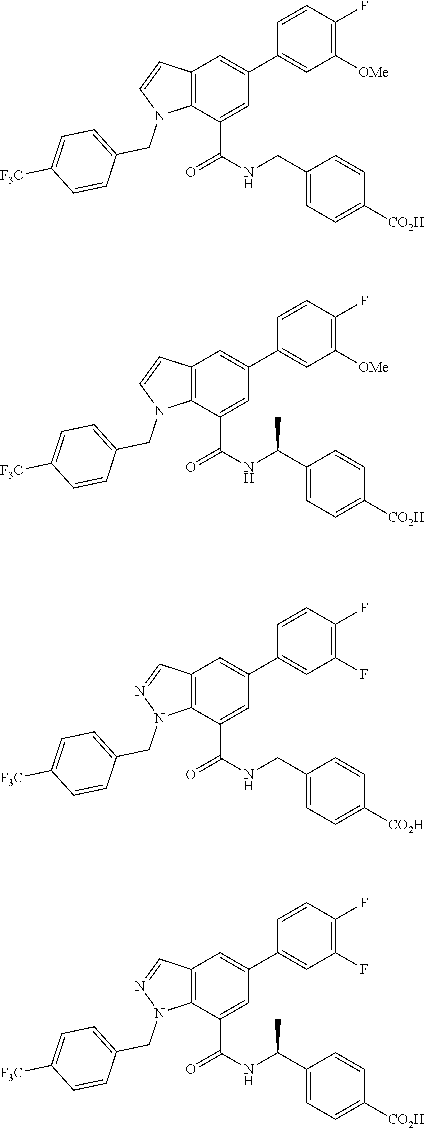 [SMILES: COc1cc(-c2cc(C(=O)NCc3ccc(C(=O)O)cc3)c3c(ccn3Cc3ccc(C(F)(F)F)cc3)c2)ccc1F ; COc1cc(-c2cc(C(=O)N[C@@H](C)c3ccc(C(=O)O)cc3)c3c(ccn3Cc3ccc(C(F)(F)F)cc3)c2)ccc1F ; O=C(O)c1ccc(CNC(=O)c2cc(-c3ccc(F)c(F)c3)cc3cnn(Cc4ccc(C(F)(F)F)cc4)c23)cc1 ; C[C@H](NC(=O)c1cc(-c2ccc(F)c(F)c2)cc2cnn(Cc3ccc(C(F)(F)F)cc3)c12)c1ccc(C(=O)O)cc1]

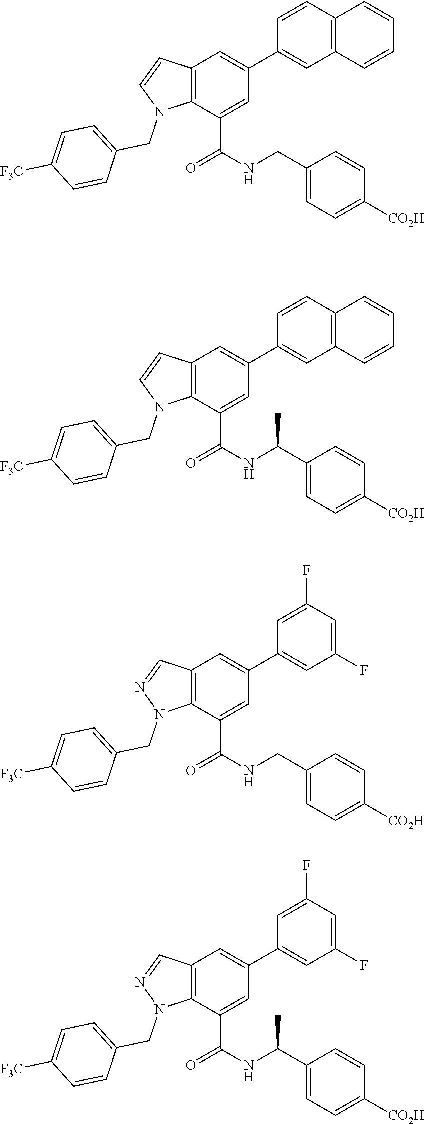 [SMILES: O=C(O)c1ccc(CNC(=O)c2cc(-c3ccc4ccccc4c3)cc3ccn(Cc4ccc(C(F)(F)F)cc4)c23)cc1 ; C[C@H](NC(=O)c1cc(-c2ccc3ccccc3c2)cc2ccn(Cc3ccc(C(F)(F)F)cc3)c12)c1ccc(C(=O)O)cc1 ; O=C(O)c1ccc(CNC(=O)c2cc(-c3cc(F)cc(F)c3)cc3cnn(Cc4ccc(C(F)(F)F)cc4)c23)cc1 ; C[C@H](NC(=O)c1cc(-c2cc(F)cc(F)c2)cc2cnn(Cc3ccc(C(F)(F)F)cc3)c12)c1ccc(C(=O)O)cc1]

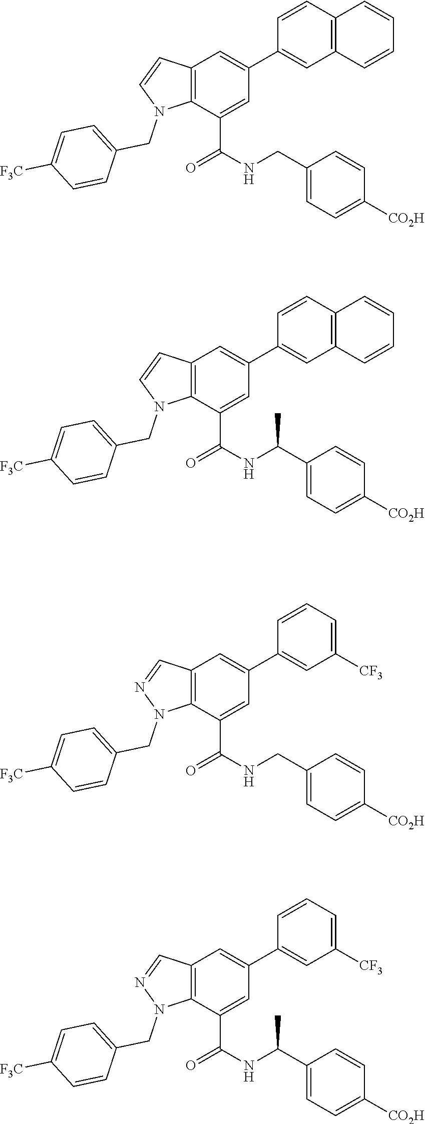 [SMILES: O=C(O)c1ccc(CNC(=O)c2cc(-c3ccc4ccccc4c3)cc3ccn(Cc4ccc(C(F)(F)F)cc4)c23)cc1 ; C[C@H](NC(=O)c1cc(-c2ccc3ccccc3c2)cc2ccn(Cc3ccc(C(F)(F)F)cc3)c12)c1ccc(C(=O)O)cc1 ; O=C(O)c1ccc(CNC(=O)c2cc(-c3cccc(C(F)(F)F)c3)cc3cnn(Cc4ccc(C(F)(F)F)cc4)c23)cc1 ; C[C@H](NC(=O)c1cc(-c2cccc(C(F)(F)F)c2)cc2cnn(Cc3ccc(C(F)(F)F)cc3)c12)c1ccc(C(=O)O)cc1]

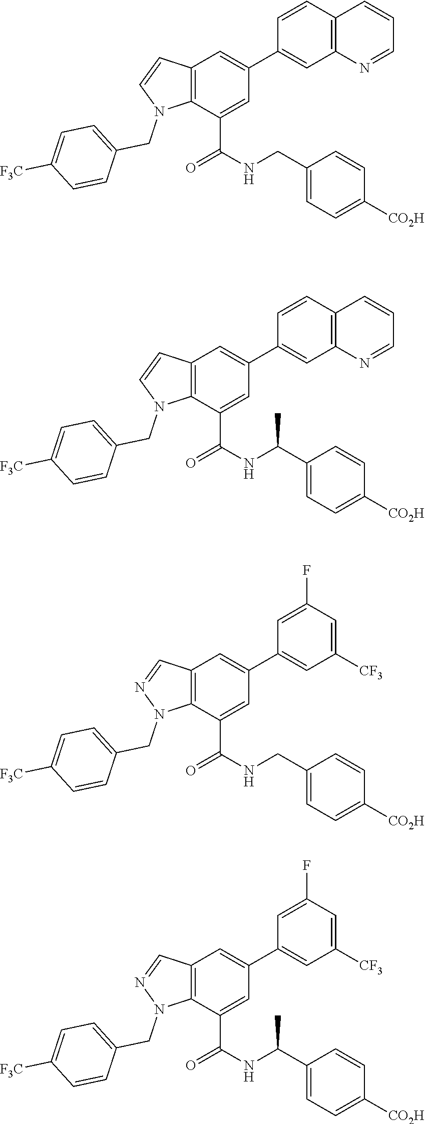 [SMILES: O=C(O)c1ccc(CNC(=O)c2cc(-c3ccc4cccnc4c3)cc3ccn(Cc4ccc(C(F)(F)F)cc4)c23)cc1 ; C[C@H](NC(=O)c1cc(-c2ccc3cccnc3c2)cc2ccn(Cc3ccc(C(F)(F)F)cc3)c12)c1ccc(C(=O)O)cc1 ; O=C(O)c1ccc(CNC(=O)c2cc(-c3cc(F)cc(C(F)(F)F)c3)cc3cnn(Cc4ccc(C(F)(F)F)cc4)c23)cc1 ; C[C@H](NC(=O)c1cc(-c2cc(F)cc(C(F)(F)F)c2)cc2cnn(Cc3ccc(C(F)(F)F)cc3)c12)c1ccc(C(=O)O)cc1]

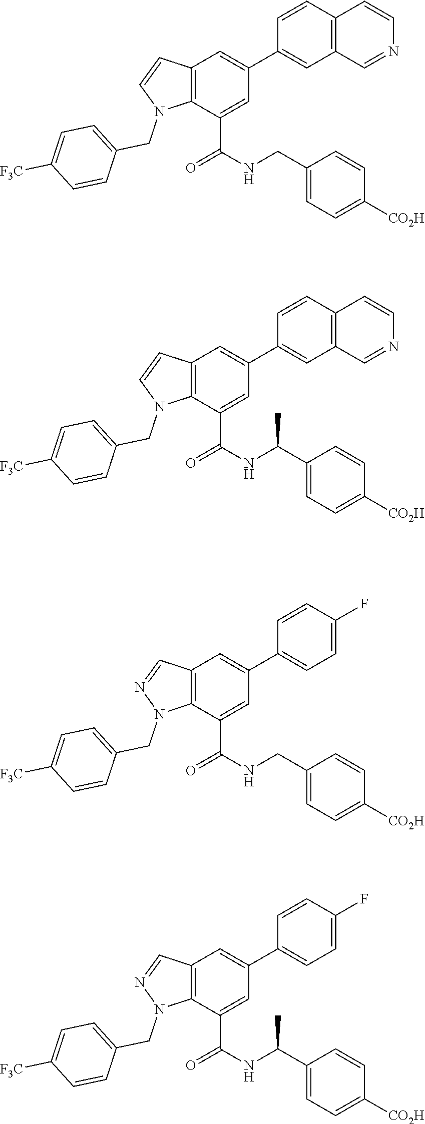 [SMILES: O=C(O)c1ccc(CNC(=O)c2cc(-c3ccc4ccncc4c3)cc3ccn(Cc4ccc(C(F)(F)F)cc4)c23)cc1 ; C[C@H](NC(=O)c1cc(-c2ccc3ccncc3c2)cc2ccn(Cc3ccc(C(F)(F)F)cc3)c12)c1ccc(C(=O)O)cc1 ; O=C(O)c1ccc(CNC(=O)c2cc(-c3ccc(F)cc3)cc3cnn(Cc4ccc(C(F)(F)F)cc4)c23)cc1 ; C[C@H](NC(=O)c1cc(-c2ccc(F)cc2)cc2cnn(Cc3ccc(C(F)(F)F)cc3)c12)c1ccc(C(=O)O)cc1]

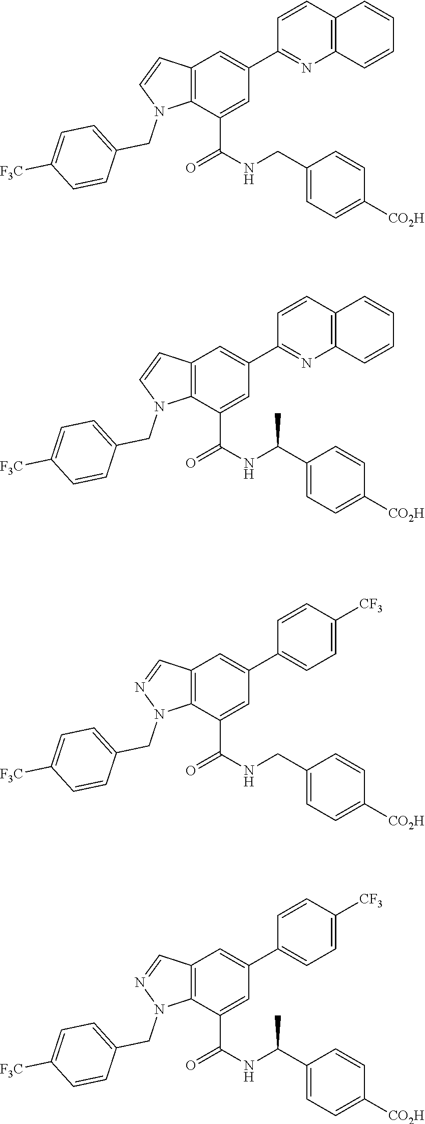 [SMILES: O=C(O)c1ccc(CNC(=O)c2cc(-c3ccc4ccccc4n3)cc3ccn(Cc4ccc(C(F)(F)F)cc4)c23)cc1 ; C[C@H](NC(=O)c1cc(-c2ccc3ccccc3n2)cc2ccn(Cc3ccc(C(F)(F)F)cc3)c12)c1ccc(C(=O)O)cc1 ; O=C(O)c1ccc(CNC(=O)c2cc(-c3ccc(C(F)(F)F)cc3)cc3cnn(Cc4ccc(C(F)(F)F)cc4)c23)cc1 ; C[C@H](NC(=O)c1cc(-c2ccc(C(F)(F)F)cc2)cc2cnn(Cc3ccc(C(F)(F)F)cc3)c12)c1ccc(C(=O)O)cc1]

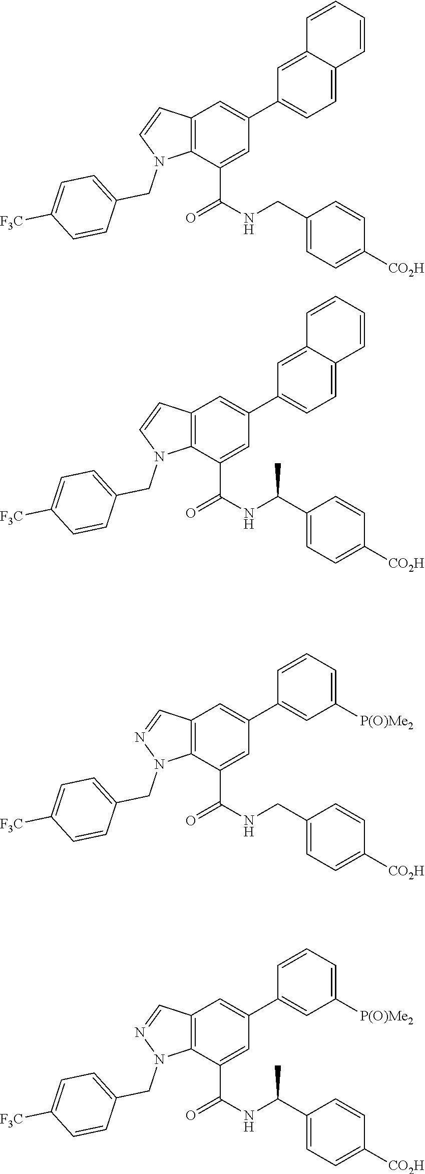

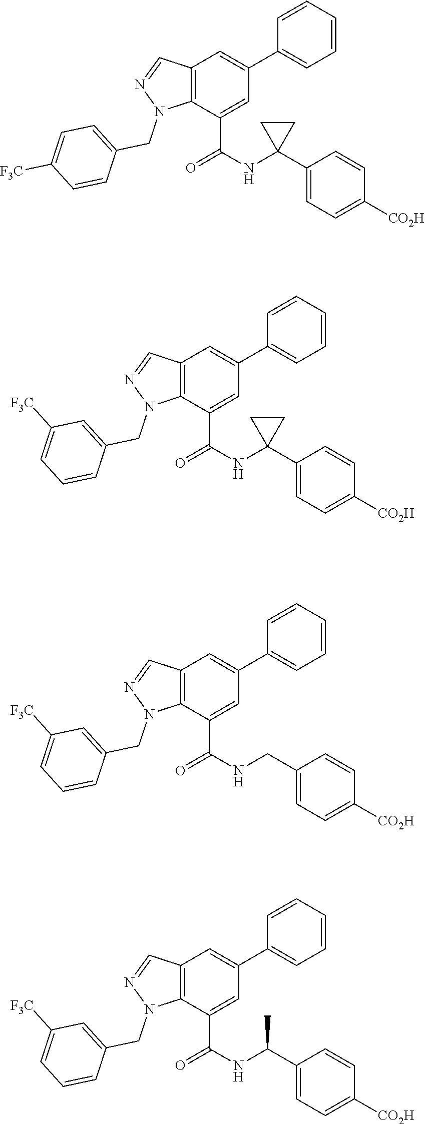 [SMILES: O=C(O)c1ccc(C2(NC(=O)c3cc(-c4ccccc4)cc4cnn(Cc5ccc(C(F)(F)F)cc5)c34)CC2)cc1 ; O=C(O)c1ccc(C2(NC(=O)c3cc(-c4ccccc4)cc4cnn(Cc5cccc(C(F)(F)F)c5)c34)CC2)cc1 ; O=C(O)c1ccc(CNC(=O)c2cc(-c3ccccc3)cc3cnn(Cc4cccc(C(F)(F)F)c4)c23)cc1 ; C[C@H](NC(=O)c1cc(-c2ccccc2)cc2cnn(Cc3cccc(C(F)(F)F)c3)c12)c1ccc(C(=O)O)cc1]

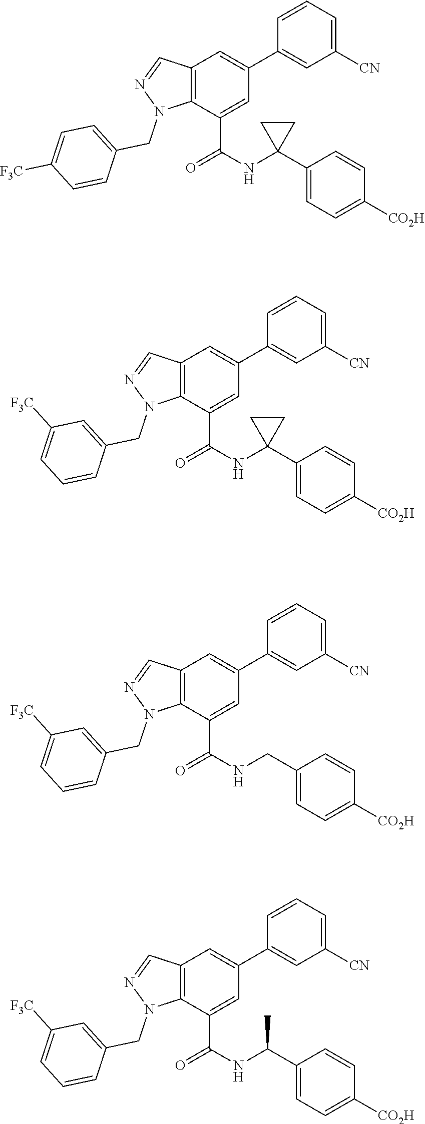 [SMILES: N#Cc1cccc(-c2cc(C(=O)NC3(c4ccc(C(=O)O)cc4)CC3)c3c(cnn3Cc3ccc(C(F)(F)F)cc3)c2)c1 ; N#Cc1cccc(-c2cc(C(=O)NC3(c4ccc(C(=O)O)cc4)CC3)c3c(cnn3Cc3cccc(C(F)(F)F)c3)c2)c1 ; N#Cc1cccc(-c2cc(C(=O)NCc3ccc(C(=O)O)cc3)c3c(cnn3Cc3cccc(C(F)(F)F)c3)c2)c1 ; C[C@H](NC(=O)c1cc(-c2cccc(C#N)c2)cc2cnn(Cc3cccc(C(F)(F)F)c3)c12)c1ccc(C(=O)O)cc1]

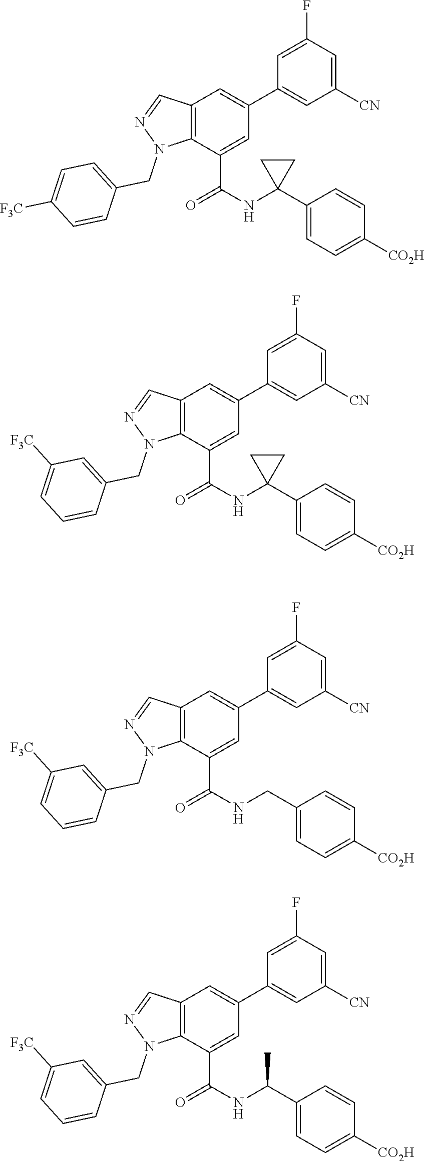 [SMILES: N#Cc1cc(F)cc(-c2cc(C(=O)NC3(c4ccc(C(=O)O)cc4)CC3)c3c(cnn3Cc3ccc(C(F)(F)F)cc3)c2)c1 ; N#Cc1cc(F)cc(-c2cc(C(=O)NC3(c4ccc(C(=O)O)cc4)CC3)c3c(cnn3Cc3cccc(C(F)(F)F)c3)c2)c1 ; N#Cc1cc(F)cc(-c2cc(C(=O)NCc3ccc(C(=O)O)cc3)c3c(cnn3Cc3cccc(C(F)(F)F)c3)c2)c1 ; C[C@H](NC(=O)c1cc(-c2cc(F)cc(C#N)c2)cc2cnn(Cc3cccc(C(F)(F)F)c3)c12)c1ccc(C(=O)O)cc1]

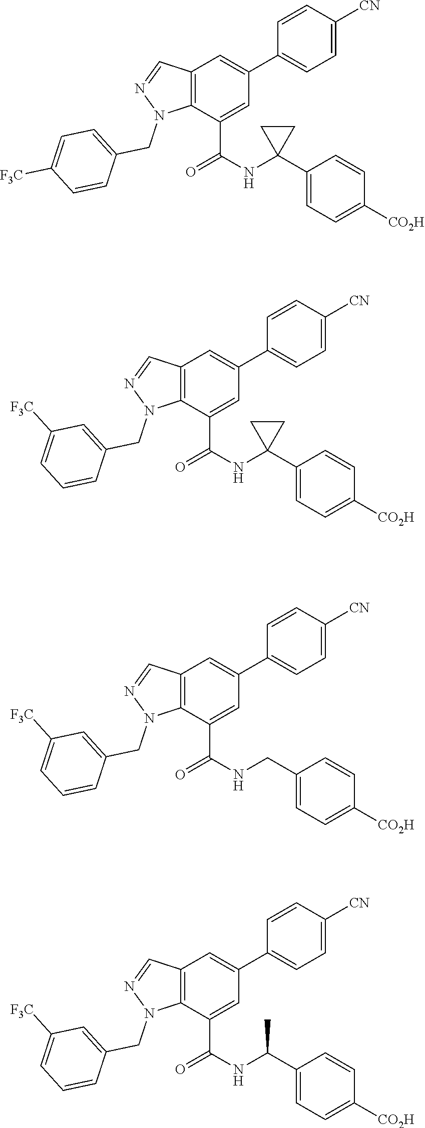 [SMILES: N#Cc1ccc(-c2cc(C(=O)NC3(c4ccc(C(=O)O)cc4)CC3)c3c(cnn3Cc3ccc(C(F)(F)F)cc3)c2)cc1 ; N#Cc1ccc(-c2cc(C(=O)NC3(c4ccc(C(=O)O)cc4)CC3)c3c(cnn3Cc3cccc(C(F)(F)F)c3)c2)cc1 ; N#Cc1ccc(-c2cc(C(=O)NCc3ccc(C(=O)O)cc3)c3c(cnn3Cc3cccc(C(F)(F)F)c3)c2)cc1 ; C[C@H](NC(=O)c1cc(-c2ccc(C#N)cc2)cc2cnn(Cc3cccc(C(F)(F)F)c3)c12)c1ccc(C(=O)O)cc1]

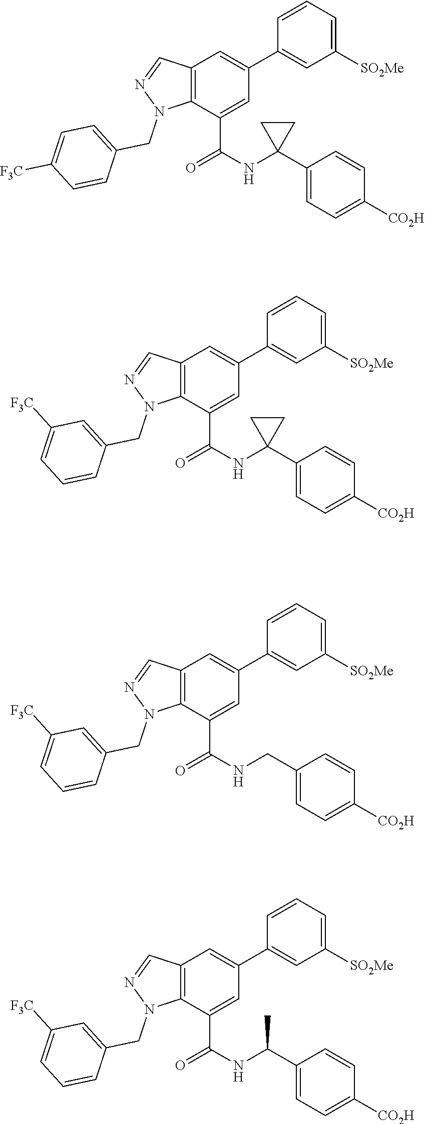 [SMILES: CS(=O)(=O)c1cccc(-c2cc(C(=O)NC3(c4ccc(C(=O)O)cc4)CC3)c3c(cnn3Cc3ccc(C(F)(F)F)cc3)c2)c1 ; CS(=O)(=O)c1cccc(-c2cc(C(=O)NC3(c4ccc(C(=O)O)cc4)CC3)c3c(cnn3Cc3cccc(C(F)(F)F)c3)c2)c1 ; CS(=O)(=O)c1cccc(-c2cc(C(=O)NCc3ccc(C(=O)O)cc3)c3c(cnn3Cc3cccc(C(F)(F)F)c3)c2)c1 ; C[C@H](NC(=O)c1cc(-c2cccc(S(C)(=O)=O)c2)cc2cnn(Cc3cccc(C(F)(F)F)c3)c12)c1ccc(C(=O)O)cc1]

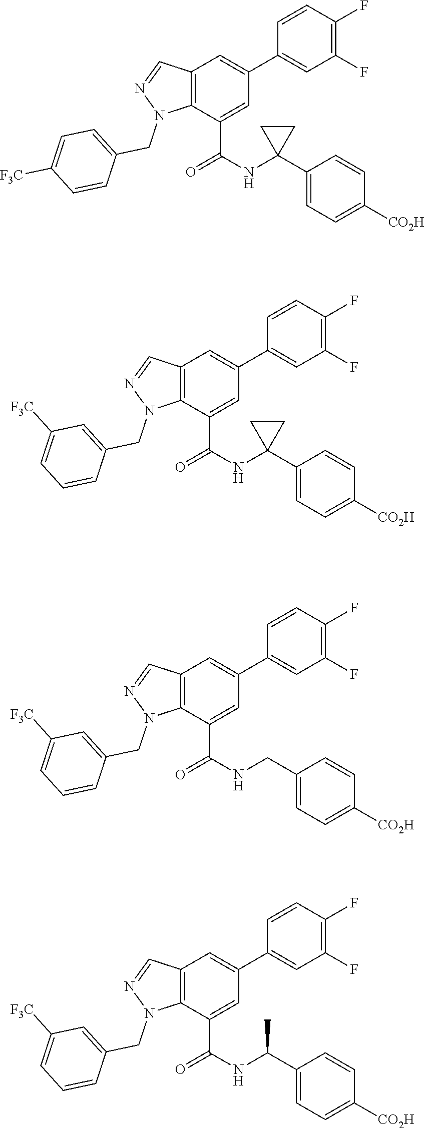 [SMILES: O=C(O)c1ccc(C2(NC(=O)c3cc(-c4ccc(F)c(F)c4)cc4cnn(Cc5ccc(C(F)(F)F)cc5)c34)CC2)cc1 ; O=C(O)c1ccc(C2(NC(=O)c3cc(-c4ccc(F)c(F)c4)cc4cnn(Cc5cccc(C(F)(F)F)c5)c34)CC2)cc1 ; O=C(O)c1ccc(CNC(=O)c2cc(-c3ccc(F)c(F)c3)cc3cnn(Cc4cccc(C(F)(F)F)c4)c23)cc1 ; C[C@H](NC(=O)c1cc(-c2ccc(F)c(F)c2)cc2cnn(Cc3cccc(C(F)(F)F)c3)c12)c1ccc(C(=O)O)cc1]

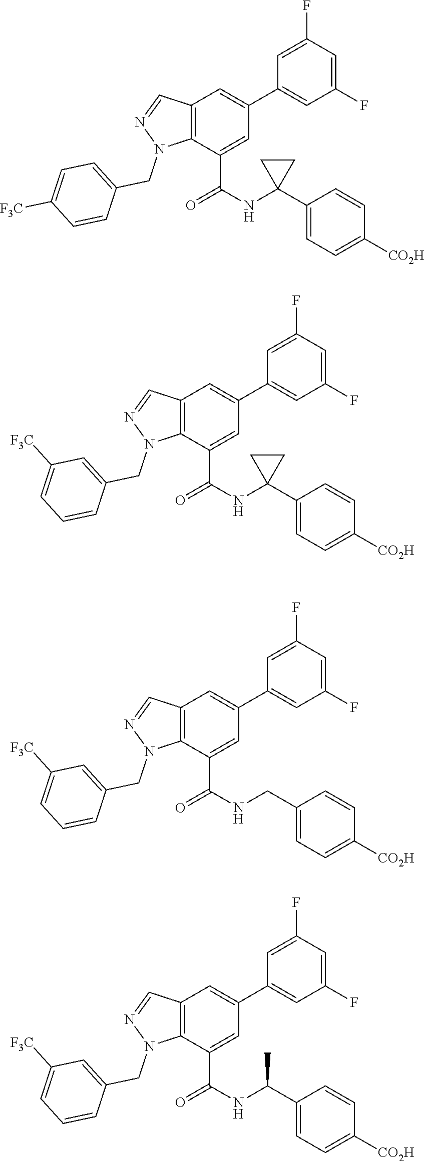 [SMILES: O=C(O)c1ccc(C2(NC(=O)c3cc(-c4cc(F)cc(F)c4)cc4cnn(Cc5ccc(C(F)(F)F)cc5)c34)CC2)cc1 ; O=C(O)c1ccc(C2(NC(=O)c3cc(-c4cc(F)cc(F)c4)cc4cnn(Cc5cccc(C(F)(F)F)c5)c34)CC2)cc1 ; O=C(O)c1ccc(CNC(=O)c2cc(-c3cc(F)cc(F)c3)cc3cnn(Cc4cccc(C(F)(F)F)c4)c23)cc1 ; C[C@H](NC(=O)c1cc(-c2cc(F)cc(F)c2)cc2cnn(Cc3cccc(C(F)(F)F)c3)c12)c1ccc(C(=O)O)cc1]

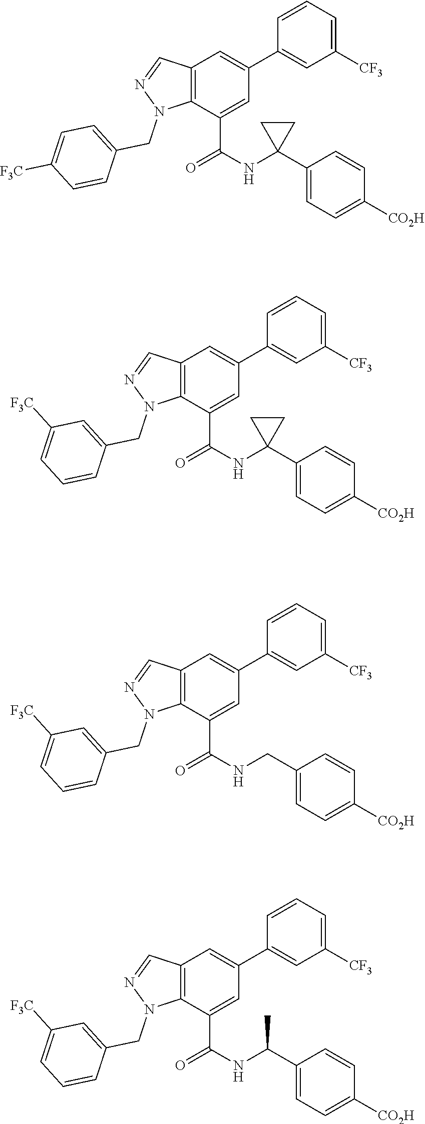 [SMILES: O=C(O)c1ccc(C2(NC(=O)c3cc(-c4cccc(C(F)(F)F)c4)cc4cnn(Cc5ccc(C(F)(F)F)cc5)c34)CC2)cc1 ; O=C(O)c1ccc(C2(NC(=O)c3cc(-c4cccc(C(F)(F)F)c4)cc4cnn(Cc5cccc(C(F)(F)F)c5)c34)CC2)cc1 ; O=C(O)c1ccc(CNC(=O)c2cc(-c3cccc(C(F)(F)F)c3)cc3cnn(Cc4cccc(C(F)(F)F)c4)c23)cc1 ; C[C@H](NC(=O)c1cc(-c2cccc(C(F)(F)F)c2)cc2cnn(Cc3cccc(C(F)(F)F)c3)c12)c1ccc(C(=O)O)cc1]

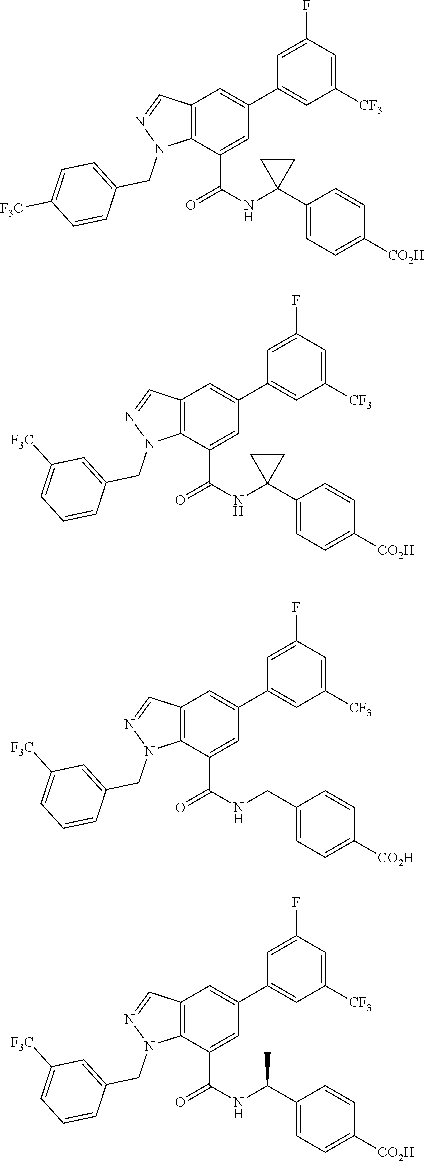 [SMILES: O=C(O)c1ccc(C2(NC(=O)c3cc(-c4cc(F)cc(C(F)(F)F)c4)cc4cnn(Cc5ccc(C(F)(F)F)cc5)c34)CC2)cc1 ; O=C(O)c1ccc(C2(NC(=O)c3cc(-c4cc(F)cc(C(F)(F)F)c4)cc4cnn(Cc5cccc(C(F)(F)F)c5)c34)CC2)cc1 ; O=C(O)c1ccc(CNC(=O)c2cc(-c3cc(F)cc(C(F)(F)F)c3)cc3cnn(Cc4cccc(C(F)(F)F)c4)c23)cc1 ; C[C@H](NC(=O)c1cc(-c2cc(F)cc(C(F)(F)F)c2)cc2cnn(Cc3cccc(C(F)(F)F)c3)c12)c1ccc(C(=O)O)cc1]

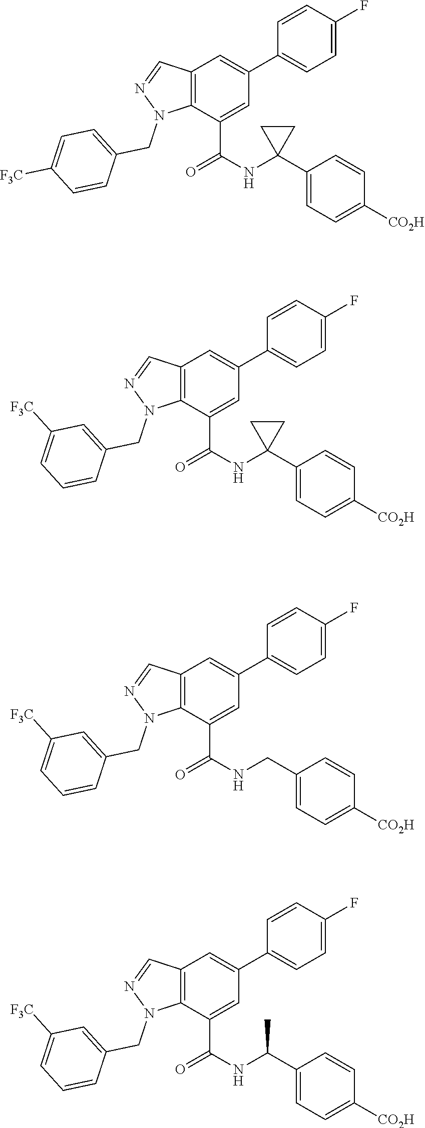 [SMILES: O=C(O)c1ccc(C2(NC(=O)c3cc(-c4ccc(F)cc4)cc4cnn(Cc5ccc(C(F)(F)F)cc5)c34)CC2)cc1 ; O=C(O)c1ccc(C2(NC(=O)c3cc(-c4ccc(F)cc4)cc4cnn(Cc5cccc(C(F)(F)F)c5)c34)CC2)cc1 ; O=C(O)c1ccc(CNC(=O)c2cc(-c3ccc(F)cc3)cc3cnn(Cc4cccc(C(F)(F)F)c4)c23)cc1 ; C[C@H](NC(=O)c1cc(-c2ccc(F)cc2)cc2cnn(Cc3cccc(C(F)(F)F)c3)c12)c1ccc(C(=O)O)cc1]

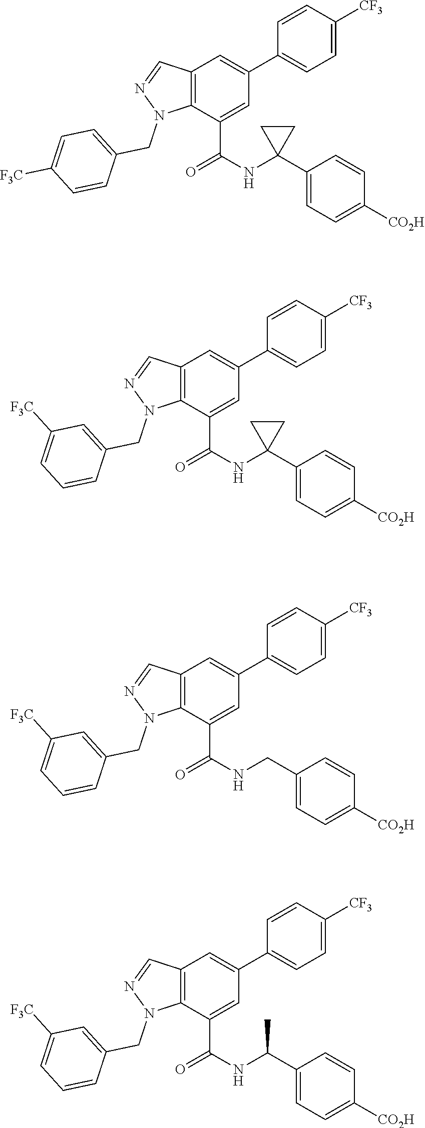 [SMILES: O=C(O)c1ccc(C2(NC(=O)c3cc(-c4ccc(C(F)(F)F)cc4)cc4cnn(Cc5ccc(C(F)(F)F)cc5)c34)CC2)cc1 ; O=C(O)c1ccc(C2(NC(=O)c3cc(-c4ccc(C(F)(F)F)cc4)cc4cnn(Cc5cccc(C(F)(F)F)c5)c34)CC2)cc1 ; O=C(O)c1ccc(CNC(=O)c2cc(-c3ccc(C(F)(F)F)cc3)cc3cnn(Cc4cccc(C(F)(F)F)c4)c23)cc1 ; C[C@H](NC(=O)c1cc(-c2ccc(C(F)(F)F)cc2)cc2cnn(Cc3cccc(C(F)(F)F)c3)c12)c1ccc(C(=O)O)cc1]

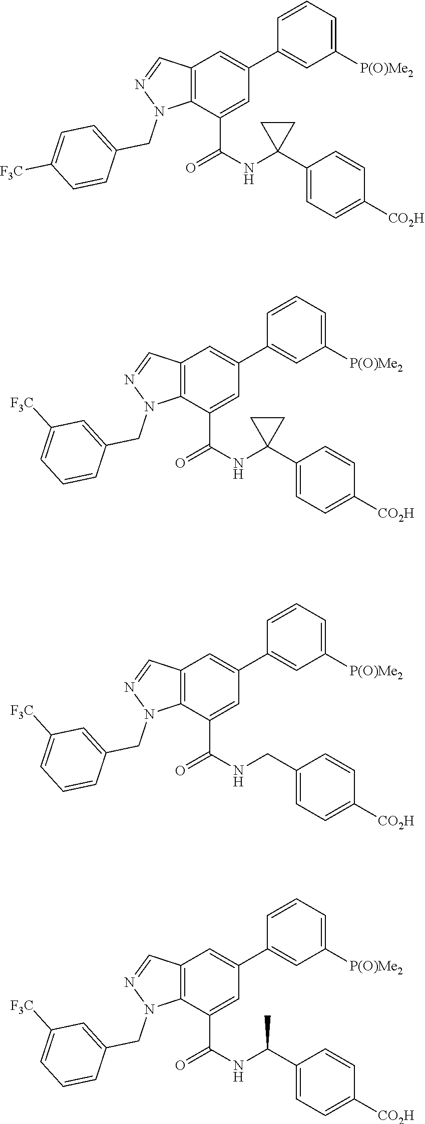

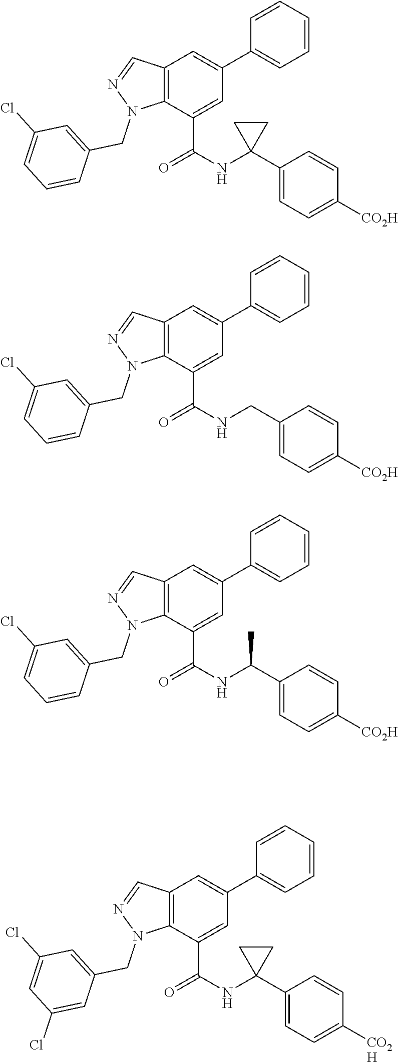 [SMILES: O=C(O)c1ccc(C2(NC(=O)c3cc(-c4ccccc4)cc4cnn(Cc5cccc(Cl)c5)c34)CC2)cc1 ; O=C(O)c1ccc(CNC(=O)c2cc(-c3ccccc3)cc3cnn(Cc4cccc(Cl)c4)c23)cc1 ; C[C@H](NC(=O)c1cc(-c2ccccc2)cc2cnn(Cc3cccc(Cl)c3)c12)c1ccc(C(=O)O)cc1 ; O=C(O)c1ccc(C2(NC(=O)c3cc(-c4ccccc4)cc4cnn(Cc5cc(Cl)cc(Cl)c5)c34)CC2)cc1]

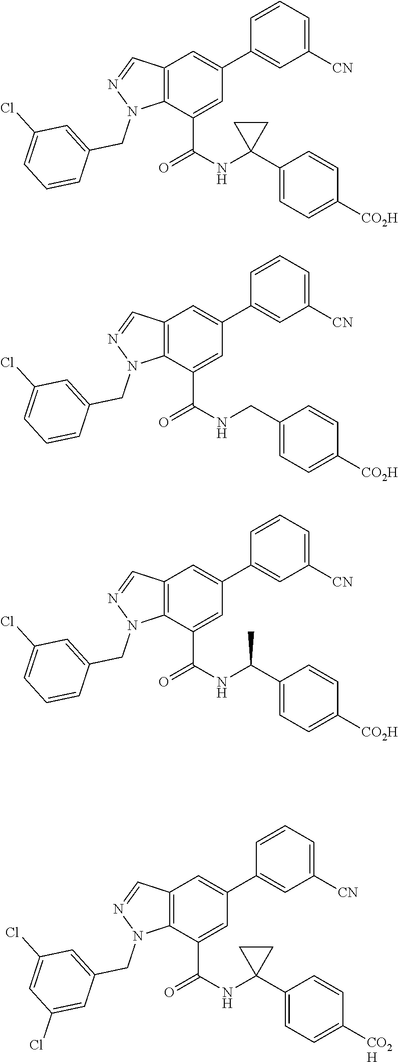 [SMILES: N#Cc1cccc(-c2cc(C(=O)NC3(c4ccc(C(=O)O)cc4)CC3)c3c(cnn3Cc3cccc(Cl)c3)c2)c1 ; N#Cc1cccc(-c2cc(C(=O)NCc3ccc(C(=O)O)cc3)c3c(cnn3Cc3cccc(Cl)c3)c2)c1 ; C[C@H](NC(=O)c1cc(-c2cccc(C#N)c2)cc2cnn(Cc3cccc(Cl)c3)c12)c1ccc(C(=O)O)cc1 ; N#Cc1cccc(-c2cc(C(=O)NC3(c4ccc(C(=O)O)cc4)CC3)c3c(cnn3Cc3cc(Cl)cc(Cl)c3)c2)c1]

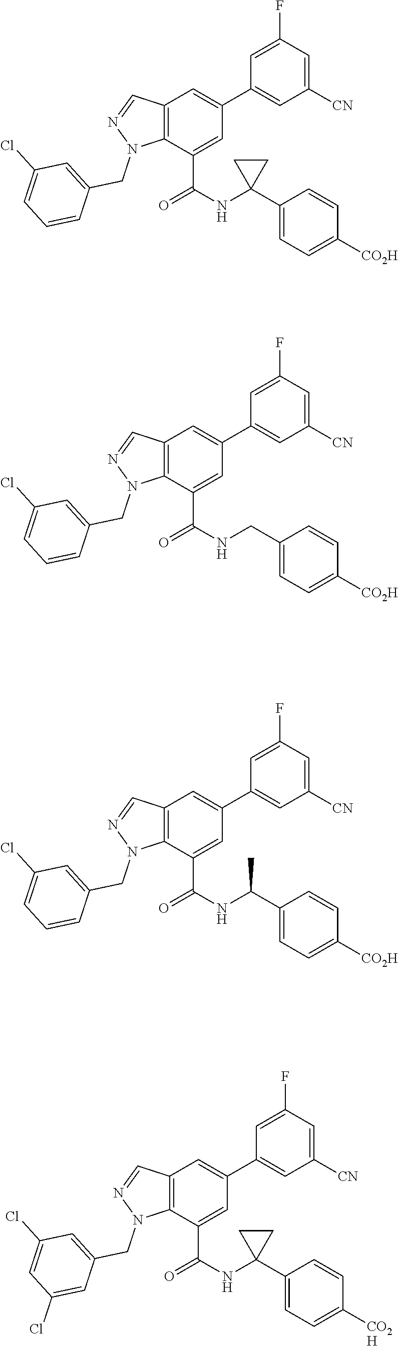 [SMILES: N#Cc1cc(F)cc(-c2cc(C(=O)NC3(c4ccc(C(=O)O)cc4)CC3)c3c(cnn3Cc3cccc(Cl)c3)c2)c1 ; N#Cc1cc(F)cc(-c2cc(C(=O)NCc3ccc(C(=O)O)cc3)c3c(cnn3Cc3cccc(Cl)c3)c2)c1 ; C[C@H](NC(=O)c1cc(-c2cc(F)cc(C#N)c2)cc2cnn(Cc3cccc(Cl)c3)c12)c1ccc(C(=O)O)cc1 ; N#Cc1cc(F)cc(-c2cc(C(=O)NC3(c4ccc(C(=O)O)cc4)CC3)c3c(cnn3Cc3cc(Cl)cc(Cl)c3)c2)c1]

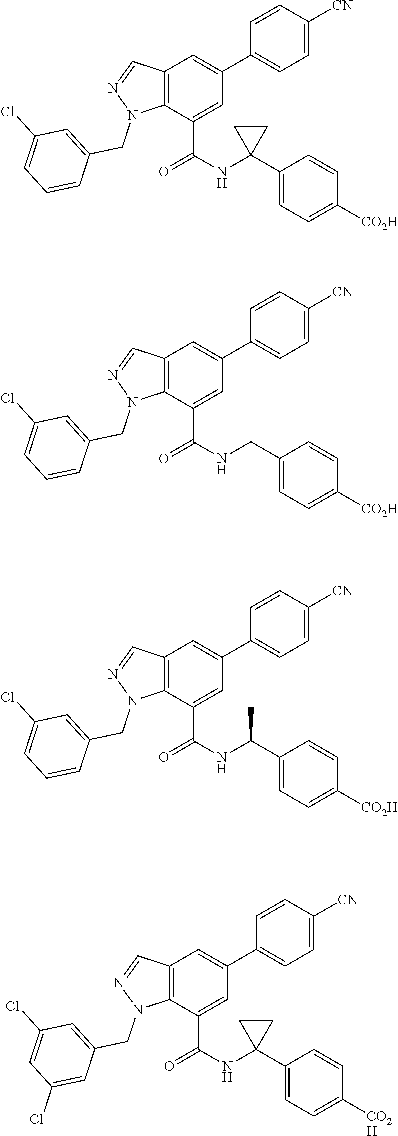 [SMILES: N#Cc1ccc(-c2cc(C(=O)NC3(c4ccc(C(=O)O)cc4)CC3)c3c(cnn3Cc3cccc(Cl)c3)c2)cc1 ; N#Cc1ccc(-c2cc(C(=O)NCc3ccc(C(=O)O)cc3)c3c(cnn3Cc3cccc(Cl)c3)c2)cc1 ; C[C@H](NC(=O)c1cc(-c2ccc(C#N)cc2)cc2cnn(Cc3cccc(Cl)c3)c12)c1ccc(C(=O)O)cc1 ; N#Cc1ccc(-c2cc(C(=O)NC3(c4ccc(C(=O)O)cc4)CC3)c3c(cnn3Cc3cc(Cl)cc(Cl)c3)c2)cc1]

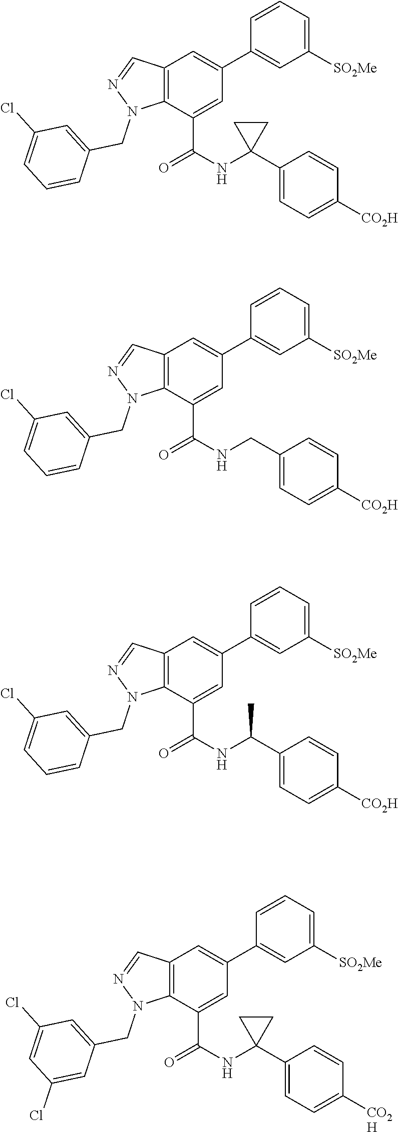 [SMILES: CS(=O)(=O)c1cccc(-c2cc(C(=O)NC3(c4ccc(C(=O)O)cc4)CC3)c3c(cnn3Cc3cccc(Cl)c3)c2)c1 ; CS(=O)(=O)c1cccc(-c2cc(C(=O)NCc3ccc(C(=O)O)cc3)c3c(cnn3Cc3cccc(Cl)c3)c2)c1 ; C[C@H](NC(=O)c1cc(-c2cccc(S(C)(=O)=O)c2)cc2cnn(Cc3cccc(Cl)c3)c12)c1ccc(C(=O)O)cc1 ; CS(=O)(=O)c1cccc(-c2cc(C(=O)NC3(c4ccc(C(=O)O)cc4)CC3)c3c(cnn3Cc3cc(Cl)cc(Cl)c3)c2)c1]

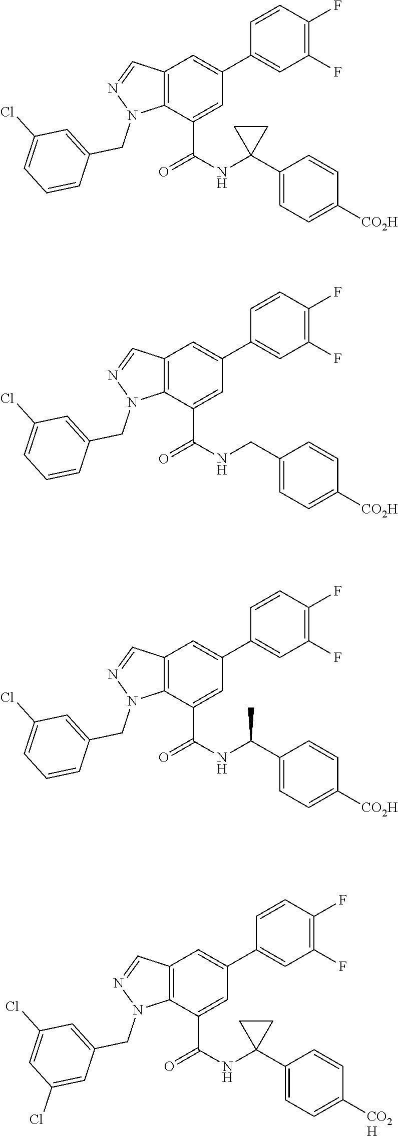 [SMILES: O=C(O)c1ccc(C2(NC(=O)c3cc(-c4ccc(F)c(F)c4)cc4cnn(Cc5cccc(Cl)c5)c34)CC2)cc1 ; O=C(O)c1ccc(CNC(=O)c2cc(-c3ccc(F)c(F)c3)cc3cnn(Cc4cccc(Cl)c4)c23)cc1 ; C[C@H](NC(=O)c1cc(-c2ccc(F)c(F)c2)cc2cnn(Cc3cccc(Cl)c3)c12)c1ccc(C(=O)O)cc1 ; O=C(O)c1ccc(C2(NC(=O)c3cc(-c4ccc(F)c(F)c4)cc4cnn(Cc5cc(Cl)cc(Cl)c5)c34)CC2)cc1]

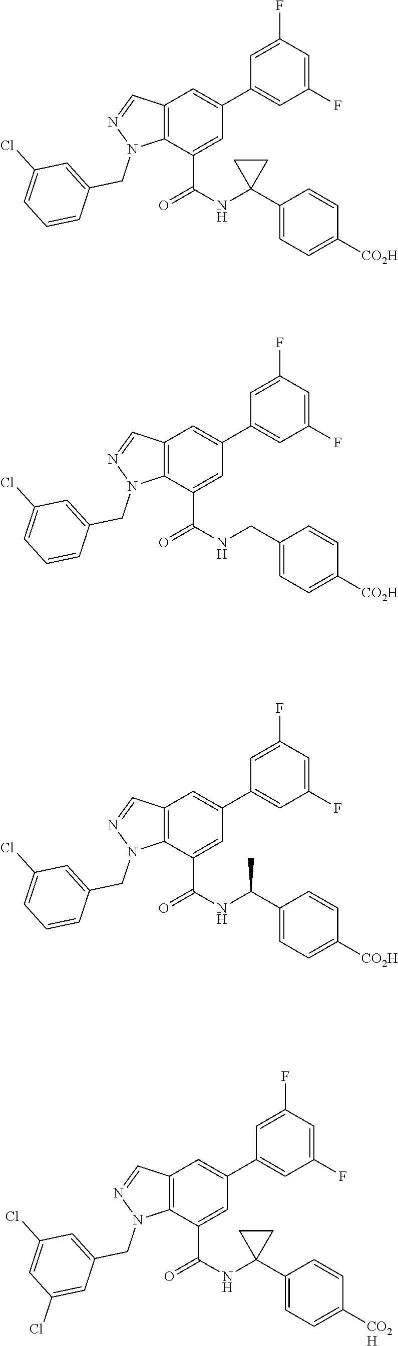 [SMILES: O=C(O)c1ccc(C2(NC(=O)c3cc(-c4cc(F)cc(F)c4)cc4cnn(Cc5cccc(Cl)c5)c34)CC2)cc1 ; O=C(O)c1ccc(CNC(=O)c2cc(-c3cc(F)cc(F)c3)cc3cnn(Cc4cccc(Cl)c4)c23)cc1 ; C[C@H](NC(=O)c1cc(-c2cc(F)cc(F)c2)cc2cnn(Cc3cccc(Cl)c3)c12)c1ccc(C(=O)O)cc1 ; O=C(O)c1ccc(C2(NC(=O)c3cc(-c4cc(F)cc(F)c4)cc4cnn(Cc5cc(Cl)cc(Cl)c5)c34)CC2)cc1]

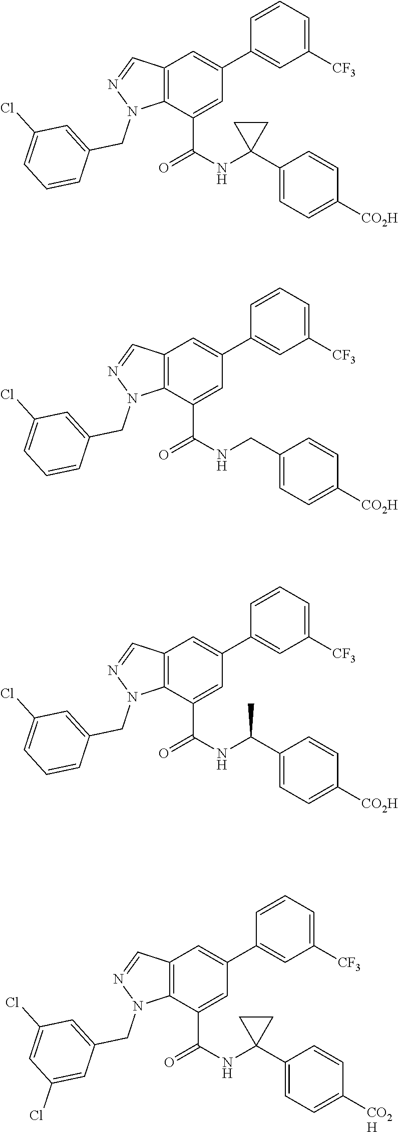 [SMILES: O=C(O)c1ccc(C2(NC(=O)c3cc(-c4cccc(C(F)(F)F)c4)cc4cnn(Cc5cccc(Cl)c5)c34)CC2)cc1 ; O=C(O)c1ccc(CNC(=O)c2cc(-c3cccc(C(F)(F)F)c3)cc3cnn(Cc4cccc(Cl)c4)c23)cc1 ; C[C@H](NC(=O)c1cc(-c2cccc(C(F)(F)F)c2)cc2cnn(Cc3cccc(Cl)c3)c12)c1ccc(C(=O)O)cc1 ; O=C(O)c1ccc(C2(NC(=O)c3cc(-c4cccc(C(F)(F)F)c4)cc4cnn(Cc5cc(Cl)cc(Cl)c5)c34)CC2)cc1]

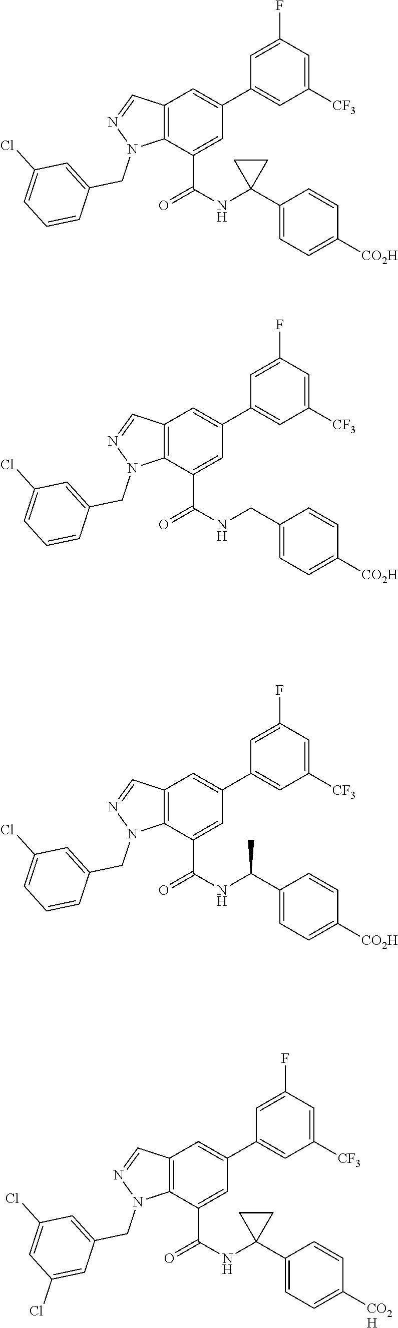 [SMILES: O=C(O)c1ccc(C2(NC(=O)c3cc(-c4cc(F)cc(C(F)(F)F)c4)cc4cnn(Cc5cccc(Cl)c5)c34)CC2)cc1 ; O=C(O)c1ccc(CNC(=O)c2cc(-c3cc(F)cc(C(F)(F)F)c3)cc3cnn(Cc4cccc(Cl)c4)c23)cc1 ; C[C@H](NC(=O)c1cc(-c2cc(F)cc(C(F)(F)F)c2)cc2cnn(Cc3cccc(Cl)c3)c12)c1ccc(C(=O)O)cc1 ; O=C(O)c1ccc(C2(NC(=O)c3cc(-c4cc(F)cc(C(F)(F)F)c4)cc4cnn(Cc5cc(Cl)cc(Cl)c5)c34)CC2)cc1]

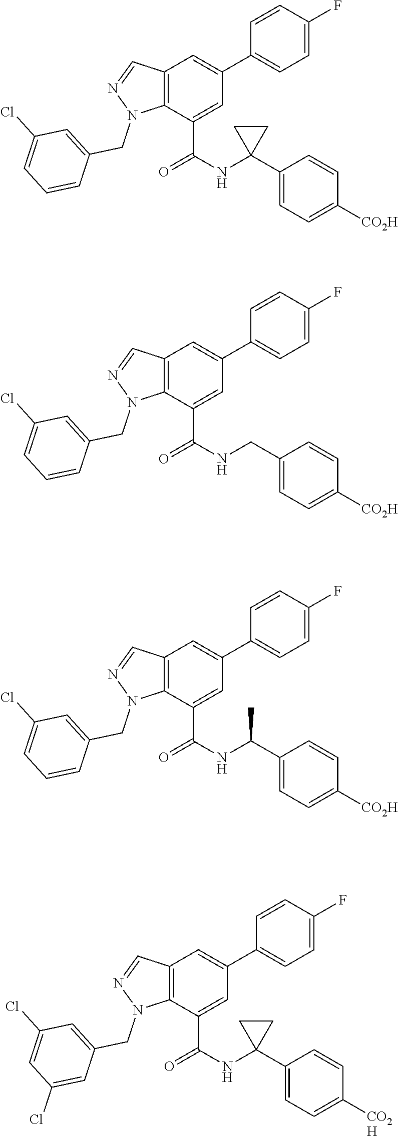 [SMILES: O=C(O)c1ccc(C2(NC(=O)c3cc(-c4ccc(F)cc4)cc4cnn(Cc5cccc(Cl)c5)c34)CC2)cc1 ; O=C(O)c1ccc(CNC(=O)c2cc(-c3ccc(F)cc3)cc3cnn(Cc4cccc(Cl)c4)c23)cc1 ; C[C@H](NC(=O)c1cc(-c2ccc(F)cc2)cc2cnn(Cc3cccc(Cl)c3)c12)c1ccc(C(=O)O)cc1 ; O=C(O)c1ccc(C2(NC(=O)c3cc(-c4ccc(F)cc4)cc4cnn(Cc5cc(Cl)cc(Cl)c5)c34)CC2)cc1]

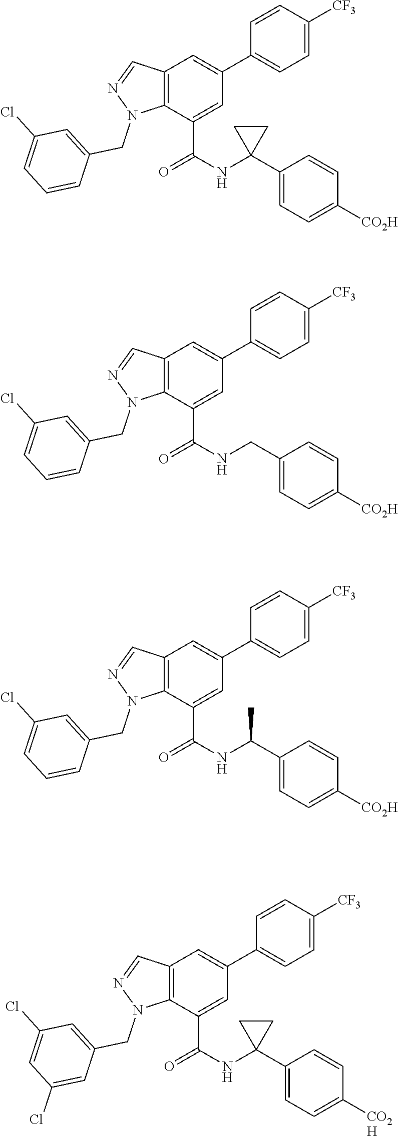 [SMILES: O=C(O)c1ccc(C2(NC(=O)c3cc(-c4ccc(C(F)(F)F)cc4)cc4cnn(Cc5cccc(Cl)c5)c34)CC2)cc1 ; O=C(O)c1ccc(CNC(=O)c2cc(-c3ccc(C(F)(F)F)cc3)cc3cnn(Cc4cccc(Cl)c4)c23)cc1 ; C[C@H](NC(=O)c1cc(-c2ccc(C(F)(F)F)cc2)cc2cnn(Cc3cccc(Cl)c3)c12)c1ccc(C(=O)O)cc1 ; O=C(O)c1ccc(C2(NC(=O)c3cc(-c4ccc(C(F)(F)F)cc4)cc4cnn(Cc5cc(Cl)cc(Cl)c5)c34)CC2)cc1]

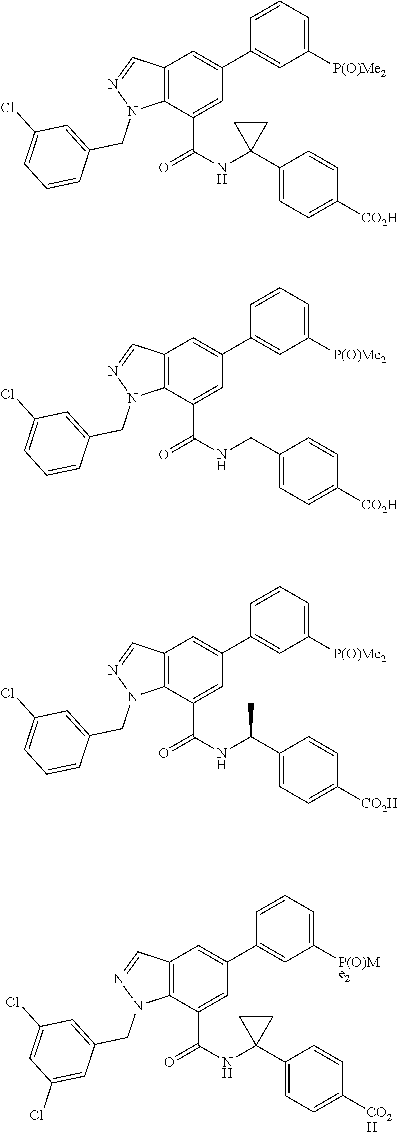

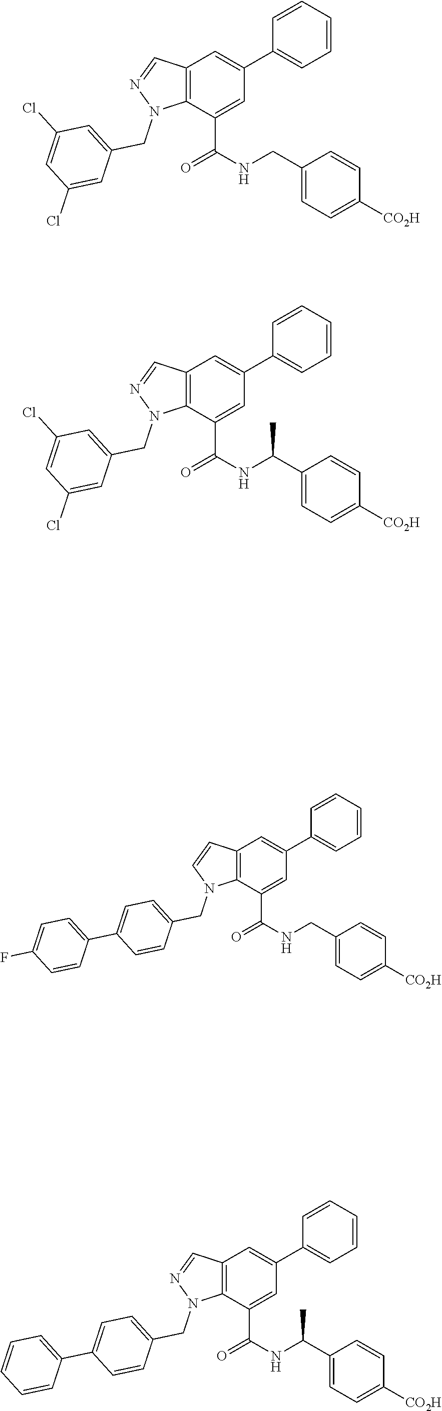 [SMILES: O=C(O)c1ccc(CNC(=O)c2cc(-c3ccccc3)cc3cnn(Cc4cc(Cl)cc(Cl)c4)c23)cc1 ; C[C@H](NC(=O)c1cc(-c2ccccc2)cc2cnn(Cc3cc(Cl)cc(Cl)c3)c12)c1ccc(C(=O)O)cc1 ; O=C(O)c1ccc(CNC(=O)c2cc(-c3ccccc3)cc3ccn(Cc4ccc(-c5ccc(F)cc5)cc4)c23)cc1 ; C[C@H](NC(=O)c1cc(-c2ccccc2)cc2cnn(Cc3ccc(-c4ccccc4)cc3)c12)c1ccc(C(=O)O)cc1]

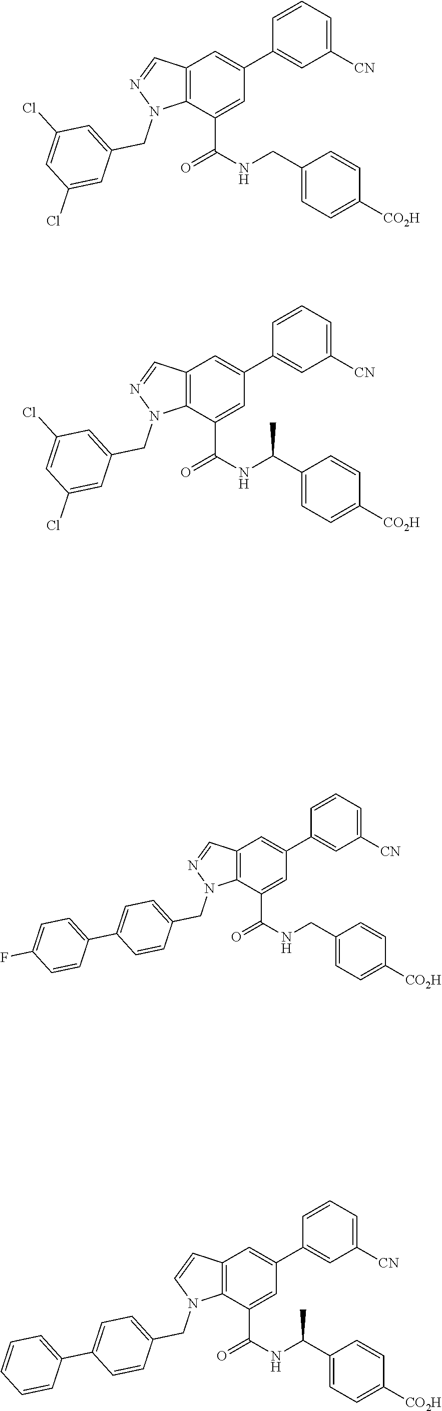 [SMILES: N#Cc1cccc(-c2cc(C(=O)NCc3ccc(C(=O)O)cc3)c3c(cnn3Cc3cc(Cl)cc(Cl)c3)c2)c1 ; C[C@H](NC(=O)c1cc(-c2cccc(C#N)c2)cc2cnn(Cc3cc(Cl)cc(Cl)c3)c12)c1ccc(C(=O)O)cc1 ; N#Cc1cccc(-c2cc(C(=O)NCc3ccc(C(=O)O)cc3)c3c(cnn3Cc3ccc(-c4ccc(F)cc4)cc3)c2)c1 ; C[C@H](NC(=O)c1cc(-c2cccc(C#N)c2)cc2ccn(Cc3ccc(-c4ccccc4)cc3)c12)c1ccc(C(=O)O)cc1]

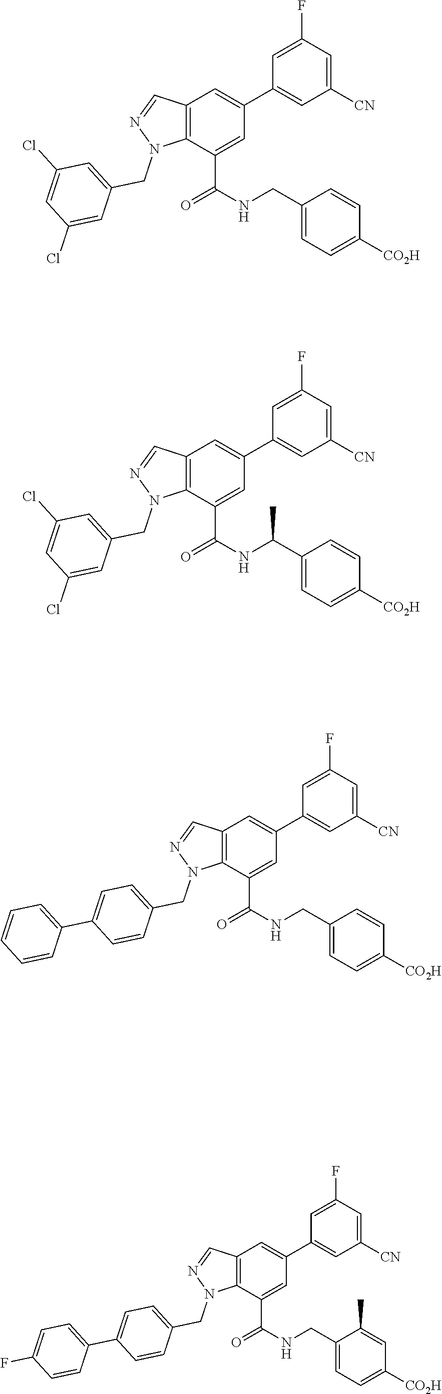

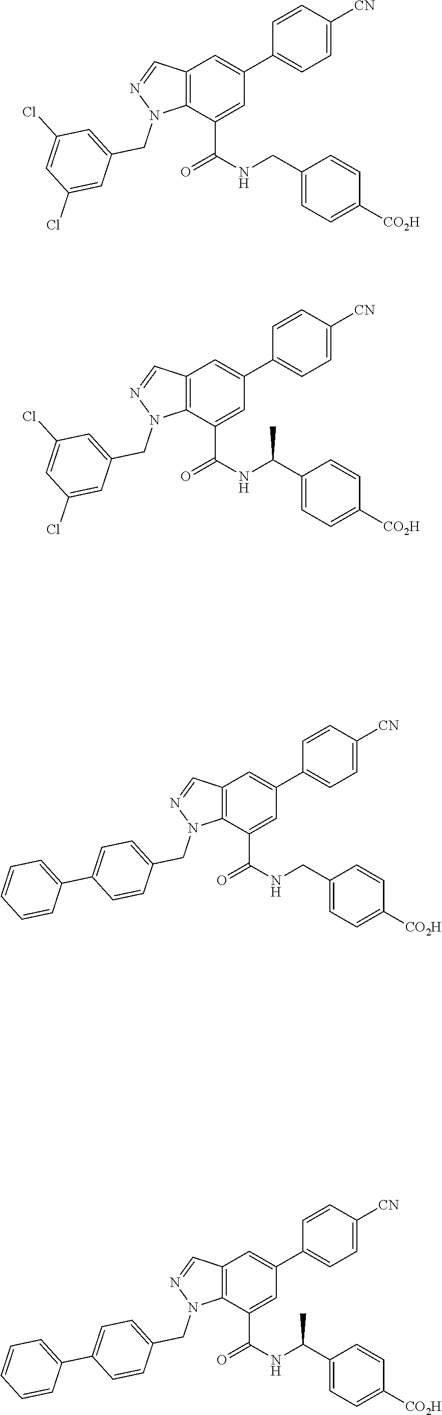 [SMILES: N#Cc1ccc(-c2cc(C(=O)NCc3ccc(C(=O)O)cc3)c3c(cnn3Cc3cc(Cl)cc(Cl)c3)c2)cc1 ; C[C@H](NC(=O)c1cc(-c2ccc(C#N)cc2)cc2cnn(Cc3cc(Cl)cc(Cl)c3)c12)c1ccc(C(=O)O)cc1 ; N#Cc1ccc(-c2cc(C(=O)NCc3ccc(C(=O)O)cc3)c3c(cnn3Cc3ccc(-c4ccccc4)cc3)c2)cc1 ; C[C@H](NC(=O)c1cc(-c2ccc(C#N)cc2)cc2cnn(Cc3ccc(-c4ccccc4)cc3)c12)c1ccc(C(=O)O)cc1]

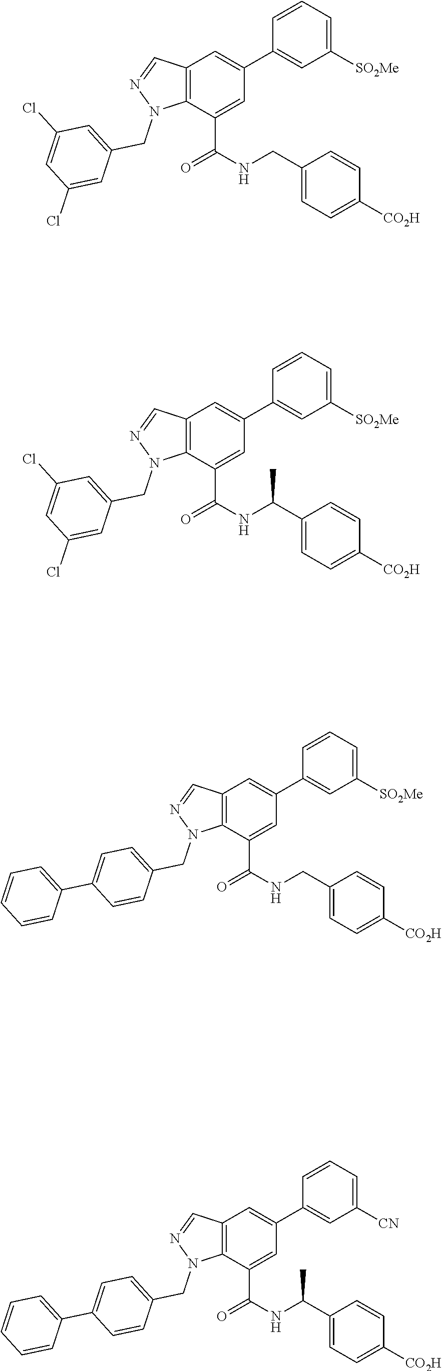 [SMILES: CS(=O)(=O)c1cccc(-c2cc(C(=O)NCc3ccc(C(=O)O)cc3)c3c(cnn3Cc3cc(Cl)cc(Cl)c3)c2)c1 ; C[C@H](NC(=O)c1cc(-c2cccc(S(C)(=O)=O)c2)cc2cnn(Cc3cc(Cl)cc(Cl)c3)c12)c1ccc(C(=O)O)cc1 ; CS(=O)(=O)c1cccc(-c2cc(C(=O)NCc3ccc(C(=O)O)cc3)c3c(cnn3Cc3ccc(-c4ccccc4)cc3)c2)c1 ; C[C@H](NC(=O)c1cc(-c2cccc(C#N)c2)cc2cnn(Cc3ccc(-c4ccccc4)cc3)c12)c1ccc(C(=O)O)cc1]

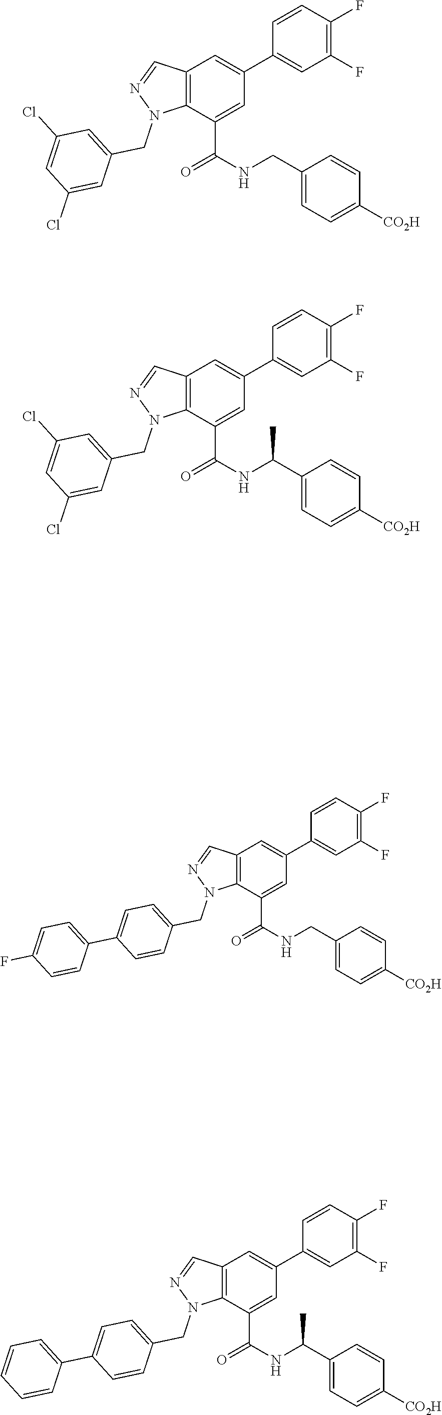 [SMILES: O=C(O)c1ccc(CNC(=O)c2cc(-c3ccc(F)c(F)c3)cc3cnn(Cc4cc(Cl)cc(Cl)c4)c23)cc1 ; C[C@H](NC(=O)c1cc(-c2ccc(F)c(F)c2)cc2cnn(Cc3cc(Cl)cc(Cl)c3)c12)c1ccc(C(=O)O)cc1 ; O=C(O)c1ccc(CNC(=O)c2cc(-c3ccc(F)c(F)c3)cc3cnn(Cc4ccc(-c5ccc(F)cc5)cc4)c23)cc1 ; C[C@H](NC(=O)c1cc(-c2ccc(F)c(F)c2)cc2cnn(Cc3ccc(-c4ccccc4)cc3)c12)c1ccc(C(=O)O)cc1]

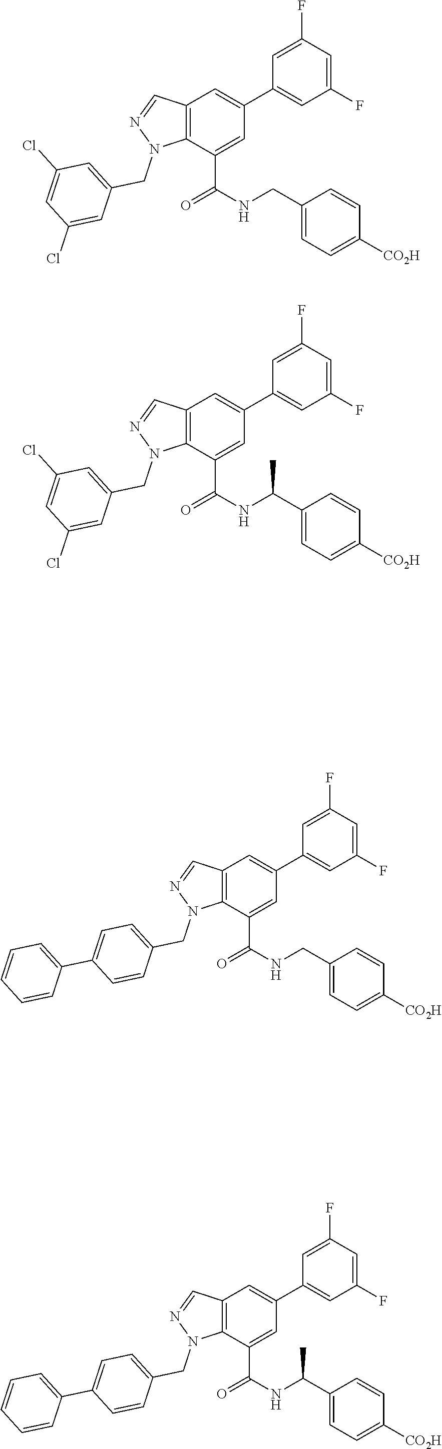 [SMILES: O=C(O)c1ccc(CNC(=O)c2cc(-c3cc(F)cc(F)c3)cc3cnn(Cc4cc(Cl)cc(Cl)c4)c23)cc1 ; C[C@H](NC(=O)c1cc(-c2cc(F)cc(F)c2)cc2cnn(Cc3cc(Cl)cc(Cl)c3)c12)c1ccc(C(=O)O)cc1 ; O=C(O)c1ccc(CNC(=O)c2cc(-c3cc(F)cc(F)c3)cc3cnn(Cc4ccc(-c5ccccc5)cc4)c23)cc1 ; C[C@H](NC(=O)c1cc(-c2cc(F)cc(F)c2)cc2cnn(Cc3ccc(-c4ccccc4)cc3)c12)c1ccc(C(=O)O)cc1]

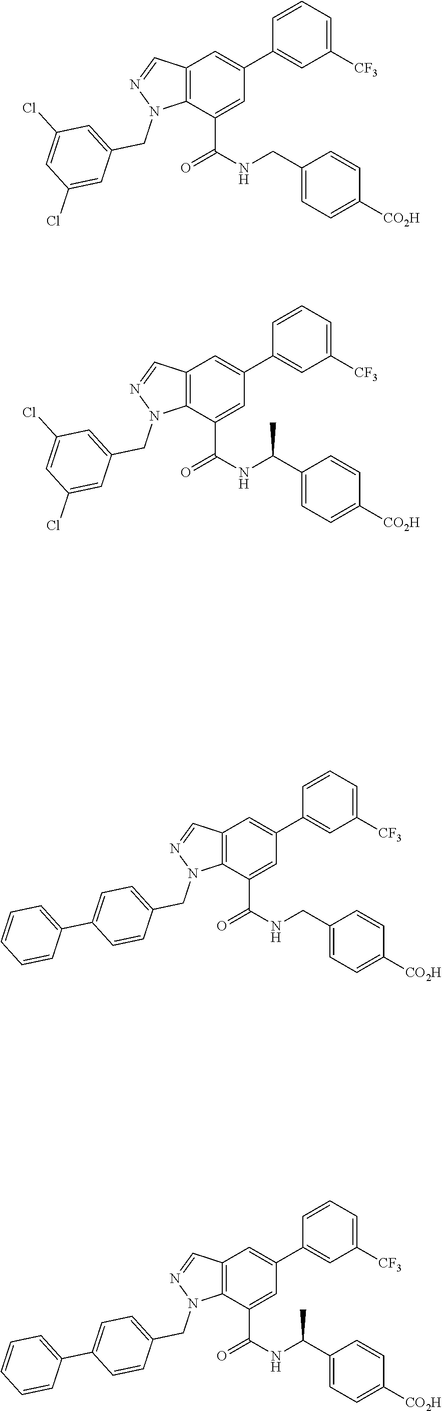 [SMILES: O=C(O)c1ccc(CNC(=O)c2cc(-c3cccc(C(F)(F)F)c3)cc3cnn(Cc4cc(Cl)cc(Cl)c4)c23)cc1 ; C[C@H](NC(=O)c1cc(-c2cccc(C(F)(F)F)c2)cc2cnn(Cc3cc(Cl)cc(Cl)c3)c12)c1ccc(C(=O)O)cc1 ; O=C(O)c1ccc(CNC(=O)c2cc(-c3cccc(C(F)(F)F)c3)cc3cnn(Cc4ccc(-c5ccccc5)cc4)c23)cc1 ; C[C@H](NC(=O)c1cc(-c2cccc(C(F)(F)F)c2)cc2cnn(Cc3ccc(-c4ccccc4)cc3)c12)c1ccc(C(=O)O)cc1]

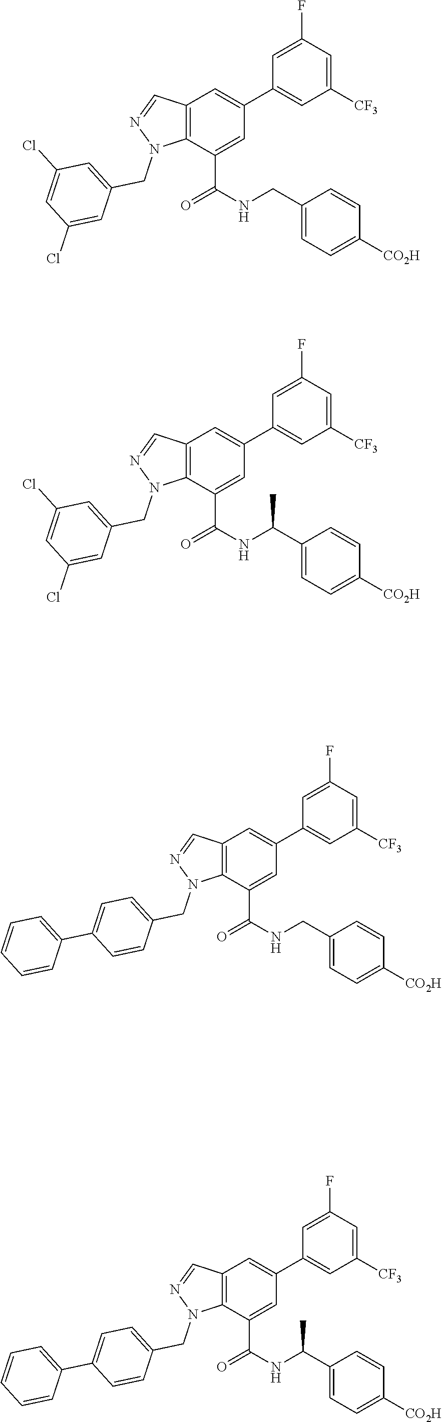 [SMILES: O=C(O)c1ccc(CNC(=O)c2cc(-c3cc(F)cc(C(F)(F)F)c3)cc3cnn(Cc4cc(Cl)cc(Cl)c4)c23)cc1 ; C[C@H](NC(=O)c1cc(-c2cc(F)cc(C(F)(F)F)c2)cc2cnn(Cc3cc(Cl)cc(Cl)c3)c12)c1ccc(C(=O)O)cc1 ; O=C(O)c1ccc(CNC(=O)c2cc(-c3cc(F)cc(C(F)(F)F)c3)cc3cnn(Cc4ccc(-c5ccccc5)cc4)c23)cc1 ; C[C@H](NC(=O)c1cc(-c2cc(F)cc(C(F)(F)F)c2)cc2cnn(Cc3ccc(-c4ccccc4)cc3)c12)c1ccc(C(=O)O)cc1]

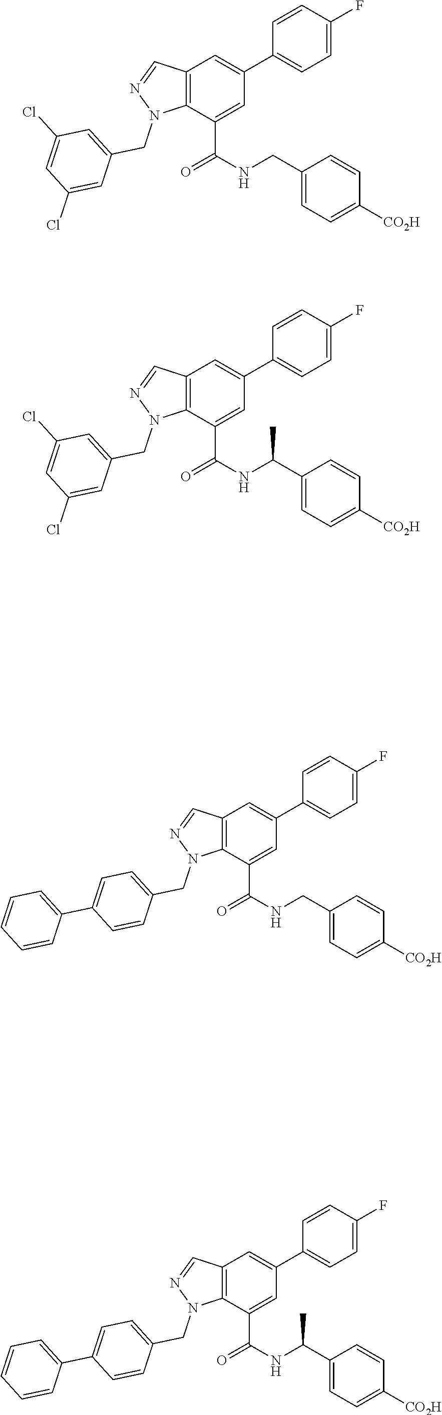 [SMILES: O=C(O)c1ccc(CNC(=O)c2cc(-c3ccc(F)cc3)cc3cnn(Cc4cc(Cl)cc(Cl)c4)c23)cc1 ; C[C@H](NC(=O)c1cc(-c2ccc(F)cc2)cc2cnn(Cc3cc(Cl)cc(Cl)c3)c12)c1ccc(C(=O)O)cc1 ; O=C(O)c1ccc(CNC(=O)c2cc(-c3ccc(F)cc3)cc3cnn(Cc4ccc(-c5ccccc5)cc4)c23)cc1 ; C[C@H](NC(=O)c1cc(-c2ccc(F)cc2)cc2cnn(Cc3ccc(-c4ccccc4)cc3)c12)c1ccc(C(=O)O)cc1]

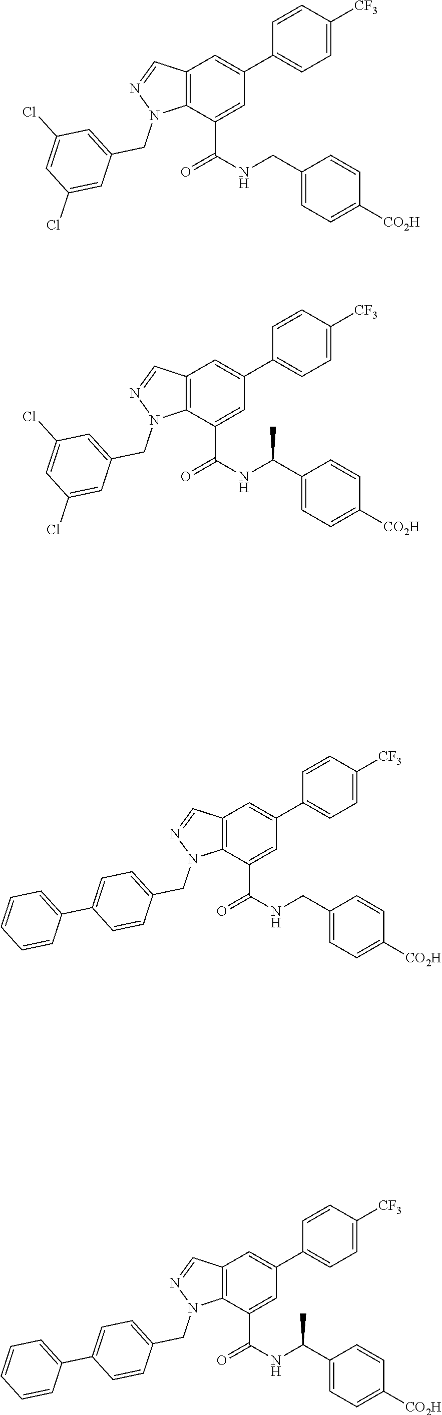 [SMILES: O=C(O)c1ccc(CNC(=O)c2cc(-c3ccc(C(F)(F)F)cc3)cc3cnn(Cc4cc(Cl)cc(Cl)c4)c23)cc1 ; C[C@H](NC(=O)c1cc(-c2ccc(C(F)(F)F)cc2)cc2cnn(Cc3cc(Cl)cc(Cl)c3)c12)c1ccc(C(=O)O)cc1 ; O=C(O)c1ccc(CNC(=O)c2cc(-c3ccc(C(F)(F)F)cc3)cc3cnn(Cc4ccc(-c5ccccc5)cc4)c23)cc1 ; C[C@H](NC(=O)c1cc(-c2ccc(C(F)(F)F)cc2)cc2cnn(Cc3ccc(-c4ccccc4)cc3)c12)c1ccc(C(=O)O)cc1]

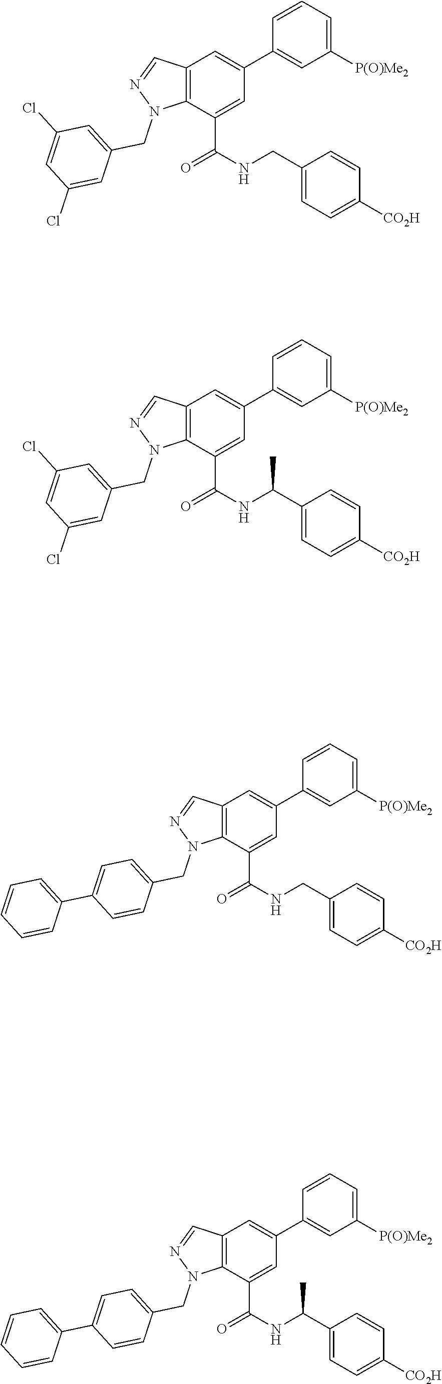

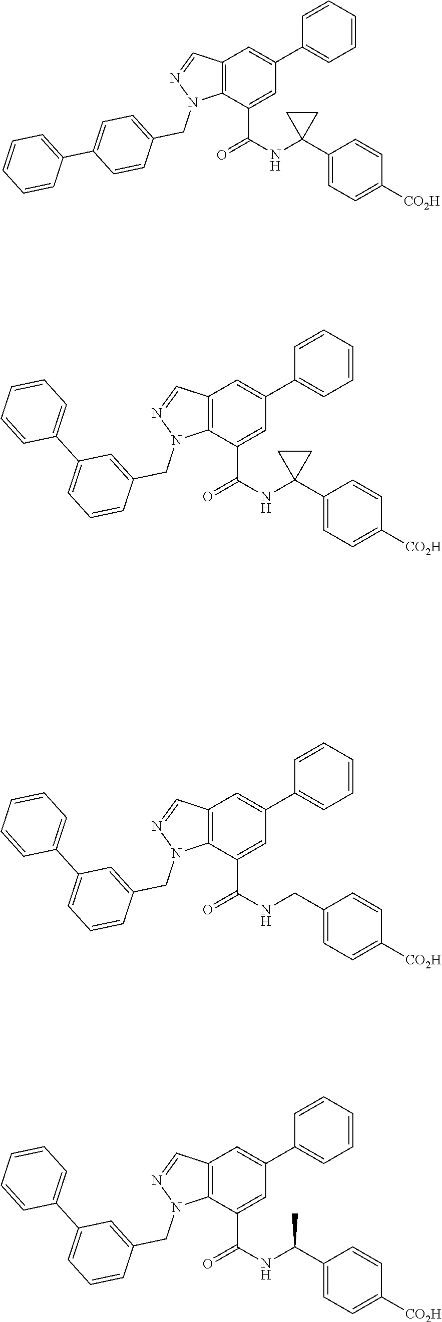 [SMILES: O=C(O)c1ccc(C2(NC(=O)c3cc(-c4ccccc4)cc4cnn(Cc5ccc(-c6ccccc6)cc5)c34)CC2)cc1 ; O=C(O)c1ccc(C2(NC(=O)c3cc(-c4ccccc4)cc4cnn(Cc5cccc(-c6ccccc6)c5)c34)CC2)cc1 ; O=C(O)c1ccc(CNC(=O)c2cc(-c3ccccc3)cc3cnn(Cc4cccc(-c5ccccc5)c4)c23)cc1 ; C[C@H](NC(=O)c1cc(-c2ccccc2)cc2cnn(Cc3cccc(-c4ccccc4)c3)c12)c1ccc(C(=O)O)cc1]

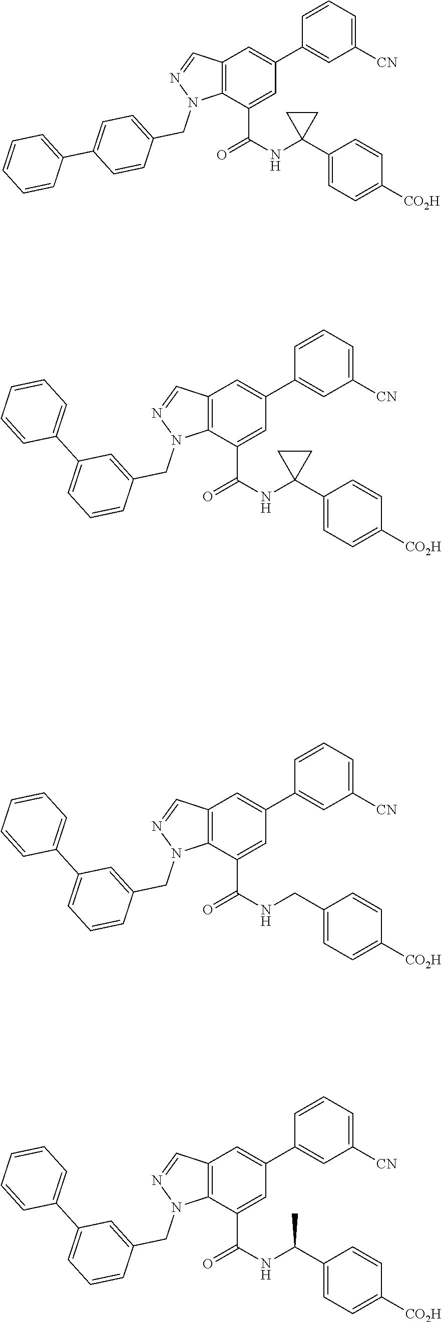 [SMILES: N#Cc1cccc(-c2cc(C(=O)NC3(c4ccc(C(=O)O)cc4)CC3)c3c(cnn3Cc3ccc(-c4ccccc4)cc3)c2)c1 ; N#Cc1cccc(-c2cc(C(=O)NC3(c4ccc(C(=O)O)cc4)CC3)c3c(cnn3Cc3cccc(-c4ccccc4)c3)c2)c1 ; N#Cc1cccc(-c2cc(C(=O)NCc3ccc(C(=O)O)cc3)c3c(cnn3Cc3cccc(-c4ccccc4)c3)c2)c1 ; C[C@H](NC(=O)c1cc(-c2cccc(C#N)c2)cc2cnn(Cc3cccc(-c4ccccc4)c3)c12)c1ccc(C(=O)O)cc1]

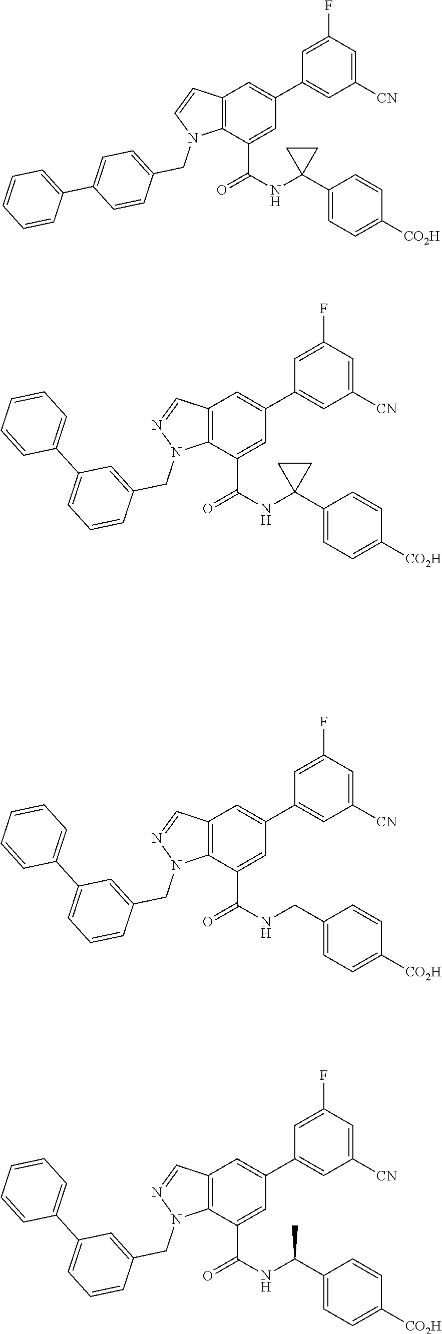 [SMILES: N#Cc1cc(F)cc(-c2cc(C(=O)NC3(c4ccc(C(=O)O)cc4)CC3)c3c(ccn3Cc3ccc(-c4ccccc4)cc3)c2)c1 ; N#Cc1cc(F)cc(-c2cc(C(=O)NC3(c4ccc(C(=O)O)cc4)CC3)c3c(cnn3Cc3cccc(-c4ccccc4)c3)c2)c1 ; N#Cc1cc(F)cc(-c2cc(C(=O)NCc3ccc(C(=O)O)cc3)c3c(cnn3Cc3cccc(-c4ccccc4)c3)c2)c1 ; C[C@H](NC(=O)c1cc(-c2cc(F)cc(C#N)c2)cc2cnn(Cc3cccc(-c4ccccc4)c3)c12)c1ccc(C(=O)O)cc1]

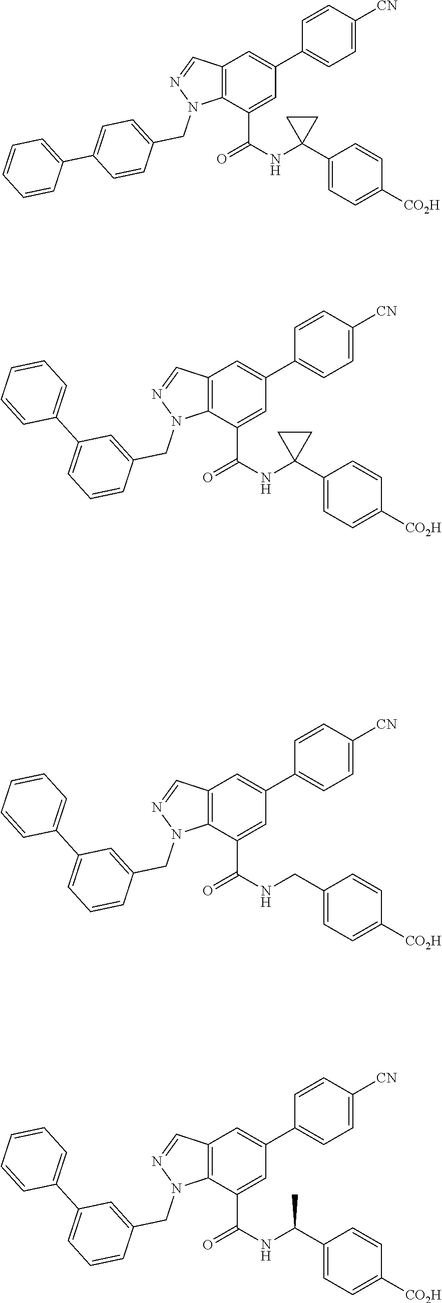 [SMILES: N#Cc1ccc(-c2cc(C(=O)NC3(c4ccc(C(=O)O)cc4)CC3)c3c(cnn3Cc3ccc(-c4ccccc4)cc3)c2)cc1 ; N#Cc1ccc(-c2cc(C(=O)NC3(c4ccc(C(=O)O)cc4)CC3)c3c(cnn3Cc3cccc(-c4ccccc4)c3)c2)cc1 ; N#Cc1ccc(-c2cc(C(=O)NCc3ccc(C(=O)O)cc3)c3c(cnn3Cc3cccc(-c4ccccc4)c3)c2)cc1 ; C[C@H](NC(=O)c1cc(-c2ccc(C#N)cc2)cc2cnn(Cc3cccc(-c4ccccc4)c3)c12)c1ccc(C(=O)O)cc1]

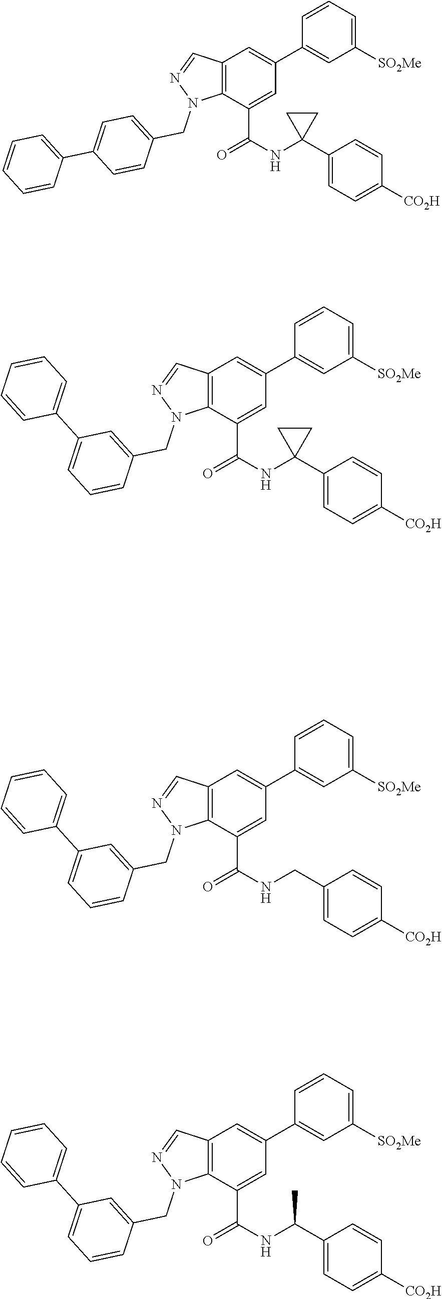 [SMILES: CS(=O)(=O)c1cccc(-c2cc(C(=O)NC3(c4ccc(C(=O)O)cc4)CC3)c3c(cnn3Cc3ccc(-c4ccccc4)cc3)c2)c1 ; CS(=O)(=O)c1cccc(-c2cc(C(=O)NC3(c4ccc(C(=O)O)cc4)CC3)c3c(cnn3Cc3cccc(-c4ccccc4)c3)c2)c1 ; CS(=O)(=O)c1cccc(-c2cc(C(=O)NCc3ccc(C(=O)O)cc3)c3c(cnn3Cc3cccc(-c4ccccc4)c3)c2)c1 ; C[C@H](NC(=O)c1cc(-c2cccc(S(C)(=O)=O)c2)cc2cnn(Cc3cccc(-c4ccccc4)c3)c12)c1ccc(C(=O)O)cc1]

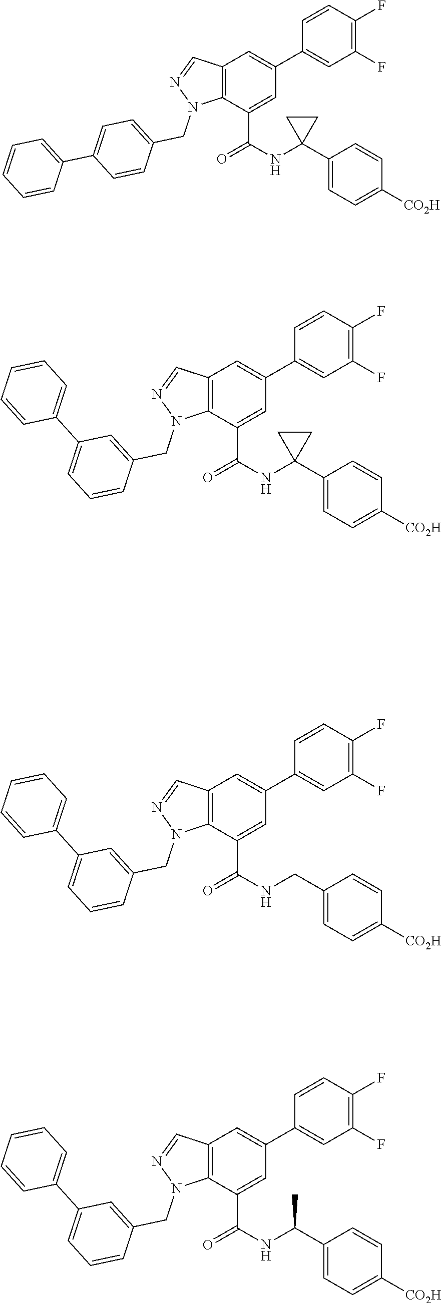 [SMILES: O=C(O)c1ccc(C2(NC(=O)c3cc(-c4ccc(F)c(F)c4)cc4cnn(Cc5ccc(-c6ccccc6)cc5)c34)CC2)cc1 ; O=C(O)c1ccc(C2(NC(=O)c3cc(-c4ccc(F)c(F)c4)cc4cnn(Cc5cccc(-c6ccccc6)c5)c34)CC2)cc1 ; O=C(O)c1ccc(CNC(=O)c2cc(-c3ccc(F)c(F)c3)cc3cnn(Cc4cccc(-c5ccccc5)c4)c23)cc1 ; C[C@H](NC(=O)c1cc(-c2ccc(F)c(F)c2)cc2cnn(Cc3cccc(-c4ccccc4)c3)c12)c1ccc(C(=O)O)cc1]

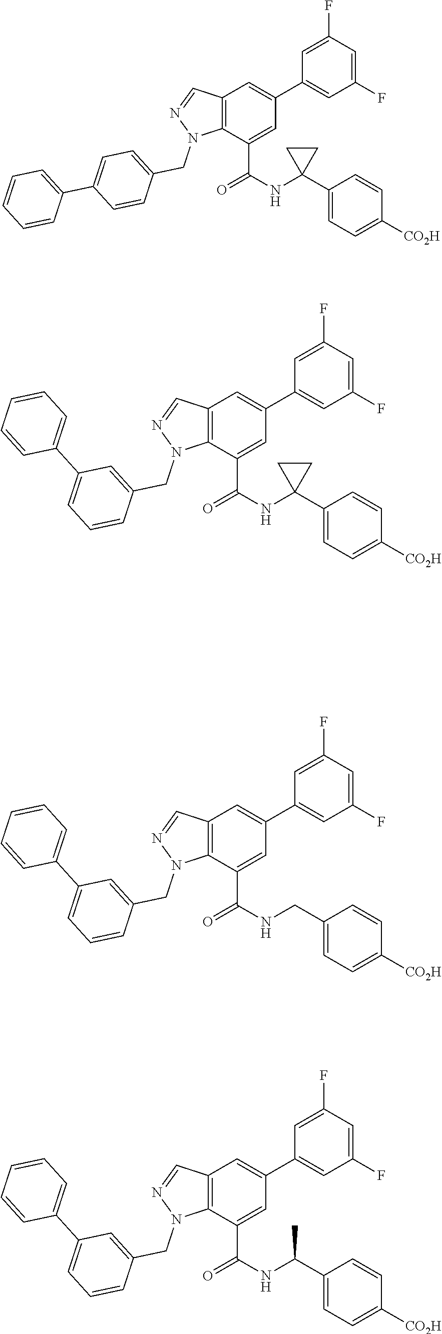 [SMILES: O=C(O)c1ccc(C2(NC(=O)c3cc(-c4cc(F)cc(F)c4)cc4cnn(Cc5ccc(-c6ccccc6)cc5)c34)CC2)cc1 ; O=C(O)c1ccc(C2(NC(=O)c3cc(-c4cc(F)cc(F)c4)cc4cnn(Cc5cccc(-c6ccccc6)c5)c34)CC2)cc1 ; O=C(O)c1ccc(CNC(=O)c2cc(-c3cc(F)cc(F)c3)cc3cnn(Cc4cccc(-c5ccccc5)c4)c23)cc1 ; C[C@H](NC(=O)c1cc(-c2cc(F)cc(F)c2)cc2cnn(Cc3cccc(-c4ccccc4)c3)c12)c1ccc(C(=O)O)cc1]

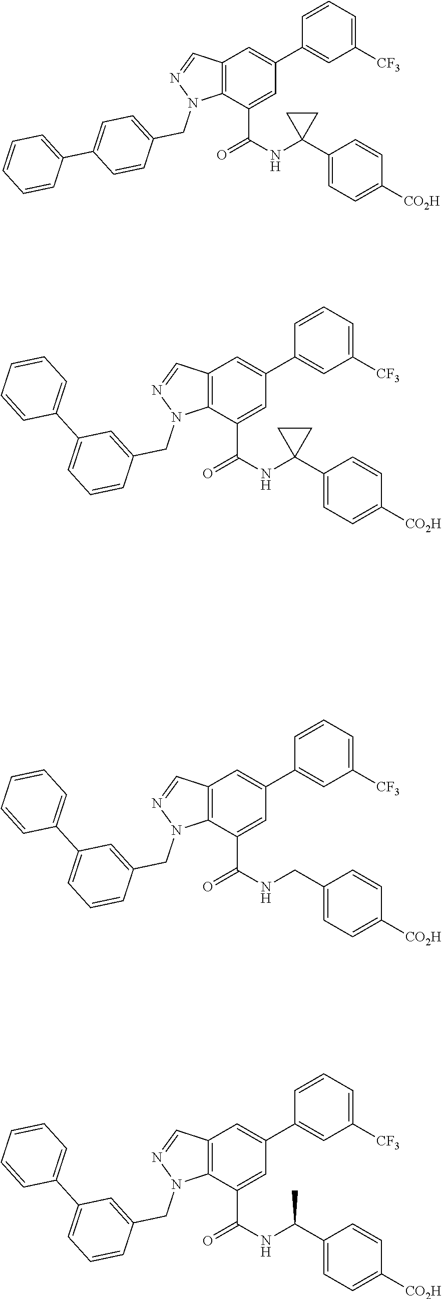 [SMILES: O=C(O)c1ccc(C2(NC(=O)c3cc(-c4cccc(C(F)(F)F)c4)cc4cnn(Cc5ccc(-c6ccccc6)cc5)c34)CC2)cc1 ; O=C(O)c1ccc(C2(NC(=O)c3cc(-c4cccc(C(F)(F)F)c4)cc4cnn(Cc5cccc(-c6ccccc6)c5)c34)CC2)cc1 ; O=C(O)c1ccc(CNC(=O)c2cc(-c3cccc(C(F)(F)F)c3)cc3cnn(Cc4cccc(-c5ccccc5)c4)c23)cc1 ; C[C@H](NC(=O)c1cc(-c2cccc(C(F)(F)F)c2)cc2cnn(Cc3cccc(-c4ccccc4)c3)c12)c1ccc(C(=O)O)cc1]

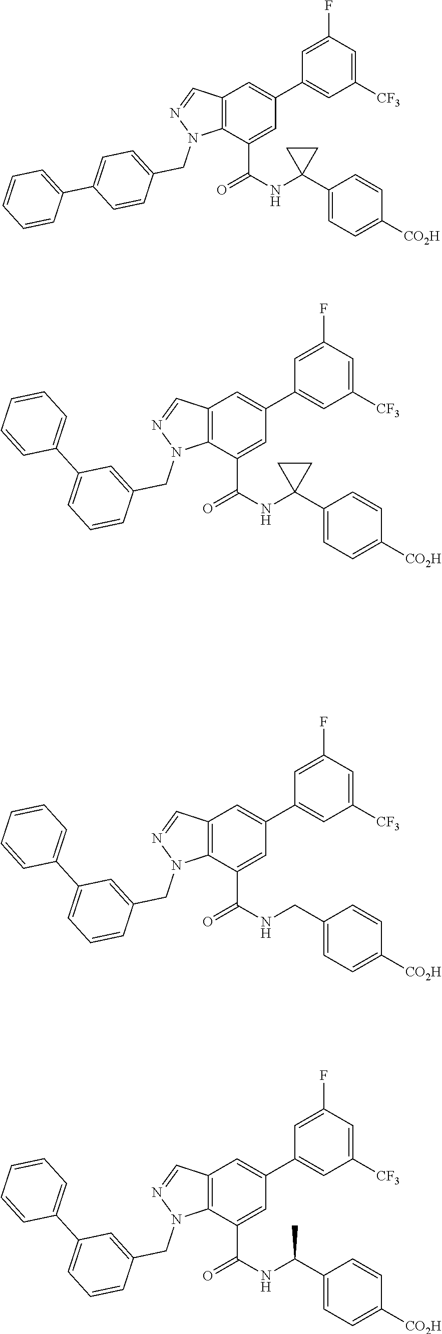 [SMILES: O=C(O)c1ccc(C2(NC(=O)c3cc(-c4cc(F)cc(C(F)(F)F)c4)cc4cnn(Cc5ccc(-c6ccccc6)cc5)c34)CC2)cc1 ; O=C(O)c1ccc(C2(NC(=O)c3cc(-c4cc(F)cc(C(F)(F)F)c4)cc4cnn(Cc5cccc(-c6ccccc6)c5)c34)CC2)cc1 ; O=C(O)c1ccc(CNC(=O)c2cc(-c3cc(F)cc(C(F)(F)F)c3)cc3cnn(Cc4cccc(-c5ccccc5)c4)c23)cc1 ; C[C@H](NC(=O)c1cc(-c2cc(F)cc(C(F)(F)F)c2)cc2cnn(Cc3cccc(-c4ccccc4)c3)c12)c1ccc(C(=O)O)cc1]

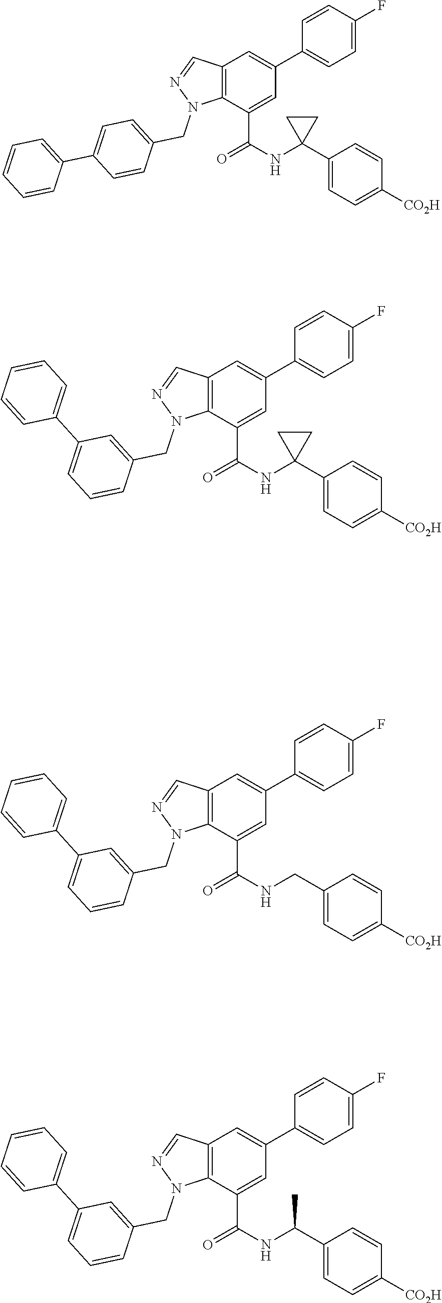 [SMILES: O=C(O)c1ccc(C2(NC(=O)c3cc(-c4ccc(F)cc4)cc4cnn(Cc5ccc(-c6ccccc6)cc5)c34)CC2)cc1 ; O=C(O)c1ccc(C2(NC(=O)c3cc(-c4ccc(F)cc4)cc4cnn(Cc5cccc(-c6ccccc6)c5)c34)CC2)cc1 ; O=C(O)c1ccc(CNC(=O)c2cc(-c3ccc(F)cc3)cc3cnn(Cc4cccc(-c5ccccc5)c4)c23)cc1 ; C[C@H](NC(=O)c1cc(-c2ccc(F)cc2)cc2cnn(Cc3cccc(-c4ccccc4)c3)c12)c1ccc(C(=O)O)cc1]

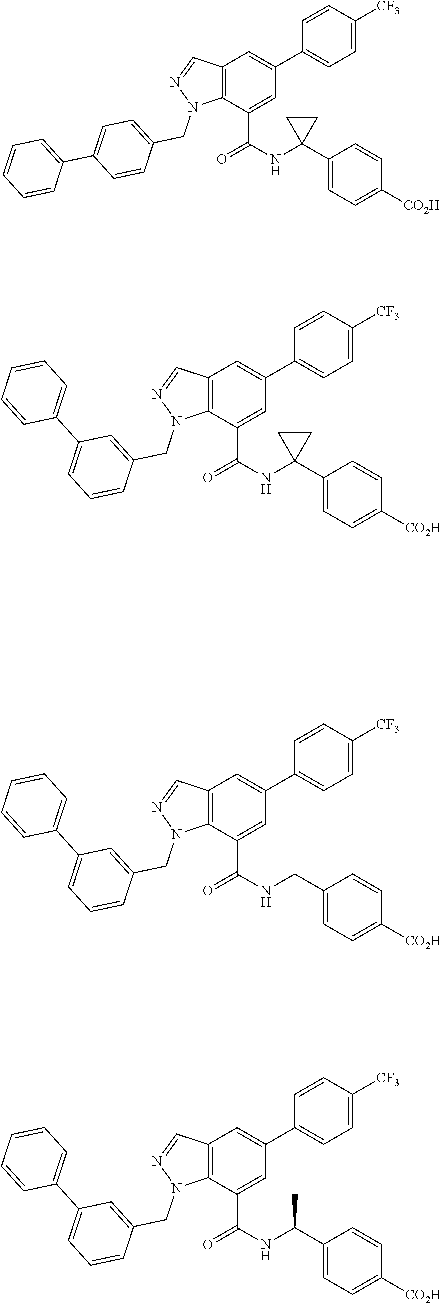 [SMILES: O=C(O)c1ccc(C2(NC(=O)c3cc(-c4ccc(C(F)(F)F)cc4)cc4cnn(Cc5ccc(-c6ccccc6)cc5)c34)CC2)cc1 ; O=C(O)c1ccc(C2(NC(=O)c3cc(-c4ccc(C(F)(F)F)cc4)cc4cnn(Cc5cccc(-c6ccccc6)c5)c34)CC2)cc1 ; O=C(O)c1ccc(CNC(=O)c2cc(-c3ccc(C(F)(F)F)cc3)cc3cnn(Cc4cccc(-c5ccccc5)c4)c23)cc1 ; C[C@H](NC(=O)c1cc(-c2ccc(C(F)(F)F)cc2)cc2cnn(Cc3cccc(-c4ccccc4)c3)c12)c1ccc(C(=O)O)cc1]

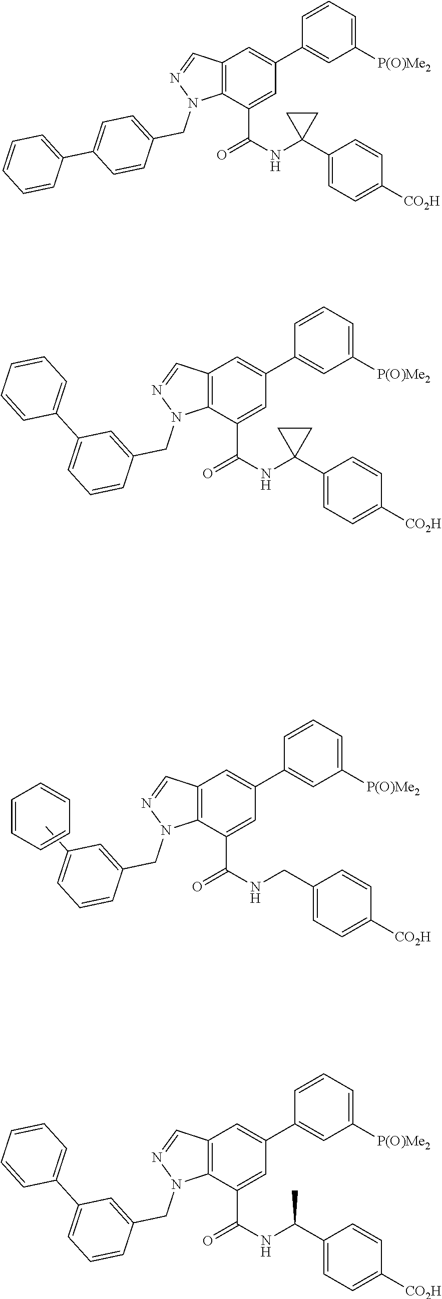

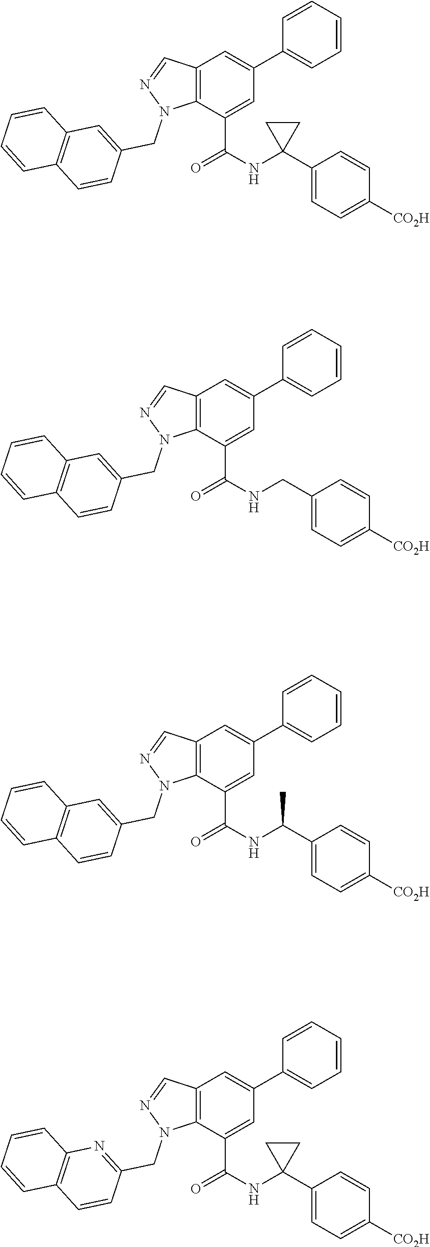 [SMILES: O=C(O)c1ccc(C2(NC(=O)c3cc(-c4ccccc4)cc4cnn(Cc5ccc6ccccc6c5)c34)CC2)cc1 ; O=C(O)c1ccc(CNC(=O)c2cc(-c3ccccc3)cc3cnn(Cc4ccc5ccccc5c4)c23)cc1 ; C[C@H](NC(=O)c1cc(-c2ccccc2)cc2cnn(Cc3ccc4ccccc4c3)c12)c1ccc(C(=O)O)cc1 ; O=C(O)c1ccc(C2(NC(=O)c3cc(-c4ccccc4)cc4cnn(Cc5ccc6ccccc6n5)c34)CC2)cc1]

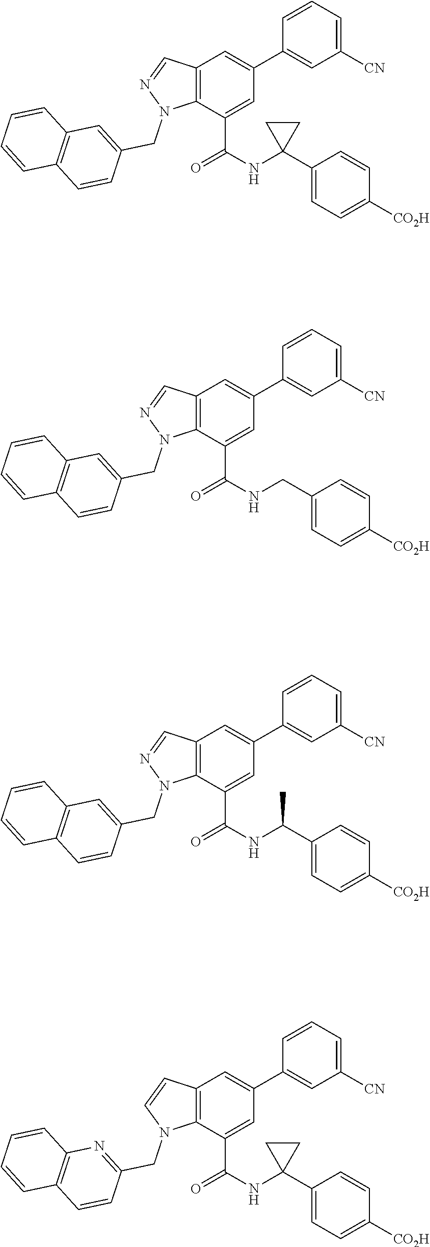 [SMILES: N#Cc1cccc(-c2cc(C(=O)NC3(c4ccc(C(=O)O)cc4)CC3)c3c(cnn3Cc3ccc4ccccc4c3)c2)c1 ; N#Cc1cccc(-c2cc(C(=O)NCc3ccc(C(=O)O)cc3)c3c(cnn3Cc3ccc4ccccc4c3)c2)c1 ; C[C@H](NC(=O)c1cc(-c2cccc(C#N)c2)cc2cnn(Cc3ccc4ccccc4c3)c12)c1ccc(C(=O)O)cc1 ; N#Cc1cccc(-c2cc(C(=O)NC3(c4ccc(C(=O)O)cc4)CC3)c3c(ccn3Cc3ccc4ccccc4n3)c2)c1]

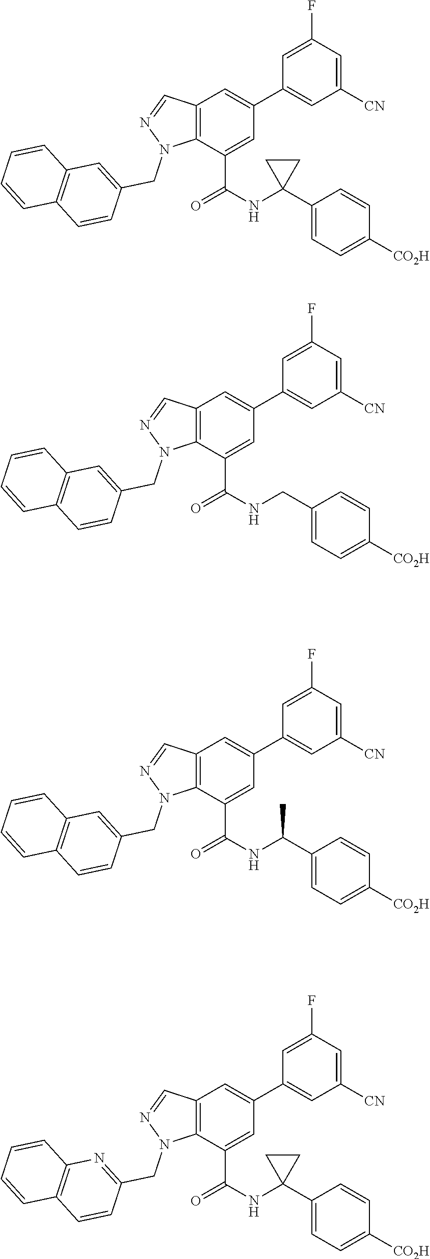 [SMILES: N#Cc1cc(F)cc(-c2cc(C(=O)NC3(c4ccc(C(=O)O)cc4)CC3)c3c(cnn3Cc3ccc4ccccc4c3)c2)c1 ; N#Cc1cc(F)cc(-c2cc(C(=O)NCc3ccc(C(=O)O)cc3)c3c(cnn3Cc3ccc4ccccc4c3)c2)c1 ; C[C@H](NC(=O)c1cc(-c2cc(F)cc(C#N)c2)cc2cnn(Cc3ccc4ccccc4c3)c12)c1ccc(C(=O)O)cc1 ; N#Cc1cc(F)cc(-c2cc(C(=O)NC3(c4ccc(C(=O)O)cc4)CC3)c3c(cnn3Cc3ccc4ccccc4n3)c2)c1]

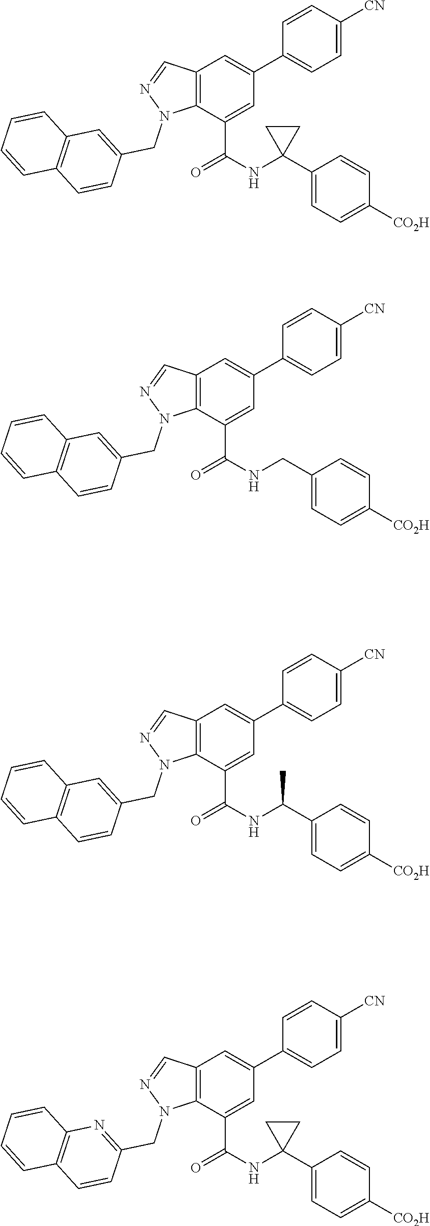 [SMILES: N#Cc1ccc(-c2cc(C(=O)NC3(c4ccc(C(=O)O)cc4)CC3)c3c(cnn3Cc3ccc4ccccc4c3)c2)cc1 ; N#Cc1ccc(-c2cc(C(=O)NCc3ccc(C(=O)O)cc3)c3c(cnn3Cc3ccc4ccccc4c3)c2)cc1 ; C[C@H](NC(=O)c1cc(-c2ccc(C#N)cc2)cc2cnn(Cc3ccc4ccccc4c3)c12)c1ccc(C(=O)O)cc1 ; N#Cc1ccc(-c2cc(C(=O)NC3(c4ccc(C(=O)O)cc4)CC3)c3c(cnn3Cc3ccc4ccccc4n3)c2)cc1]

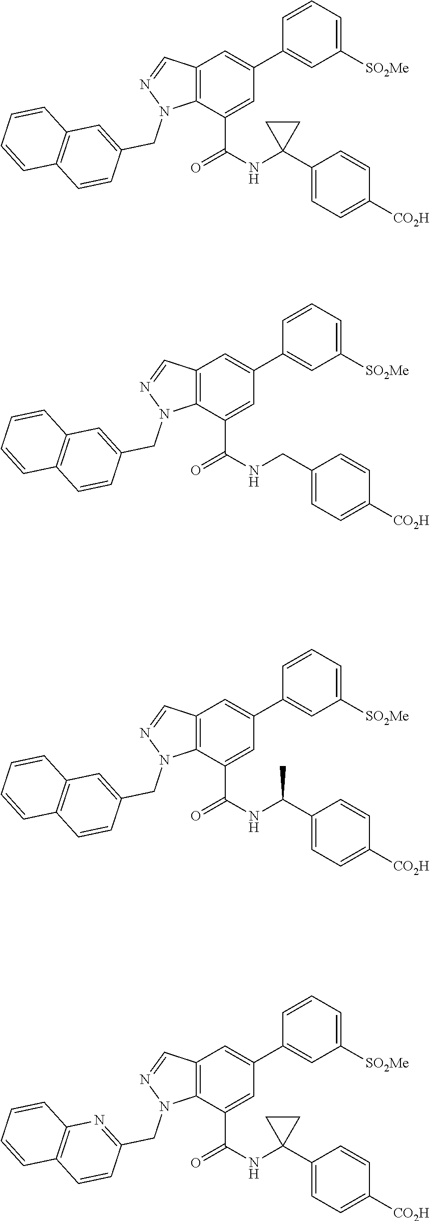 [SMILES: CS(=O)(=O)c1cccc(-c2cc(C(=O)NC3(c4ccc(C(=O)O)cc4)CC3)c3c(cnn3Cc3ccc4ccccc4c3)c2)c1 ; CS(=O)(=O)c1cccc(-c2cc(C(=O)NCc3ccc(C(=O)O)cc3)c3c(cnn3Cc3ccc4ccccc4c3)c2)c1 ; C[C@H](NC(=O)c1cc(-c2cccc(S(C)(=O)=O)c2)cc2cnn(Cc3ccc4ccccc4c3)c12)c1ccc(C(=O)O)cc1 ; CS(=O)(=O)c1cccc(-c2cc(C(=O)NC3(c4ccc(C(=O)O)cc4)CC3)c3c(cnn3Cc3ccc4ccccc4n3)c2)c1]

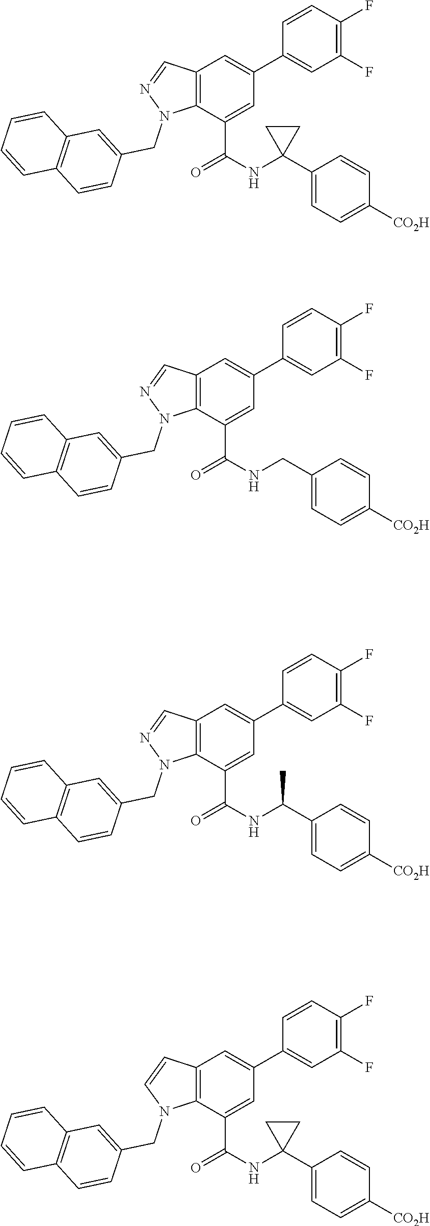 [SMILES: O=C(O)c1ccc(C2(NC(=O)c3cc(-c4ccc(F)c(F)c4)cc4cnn(Cc5ccc6ccccc6c5)c34)CC2)cc1 ; O=C(O)c1ccc(CNC(=O)c2cc(-c3ccc(F)c(F)c3)cc3cnn(Cc4ccc5ccccc5c4)c23)cc1 ; C[C@H](NC(=O)c1cc(-c2ccc(F)c(F)c2)cc2cnn(Cc3ccc4ccccc4c3)c12)c1ccc(C(=O)O)cc1 ; O=C(O)c1ccc(C2(NC(=O)c3cc(-c4ccc(F)c(F)c4)cc4ccn(Cc5ccc6ccccc6c5)c34)CC2)cc1]

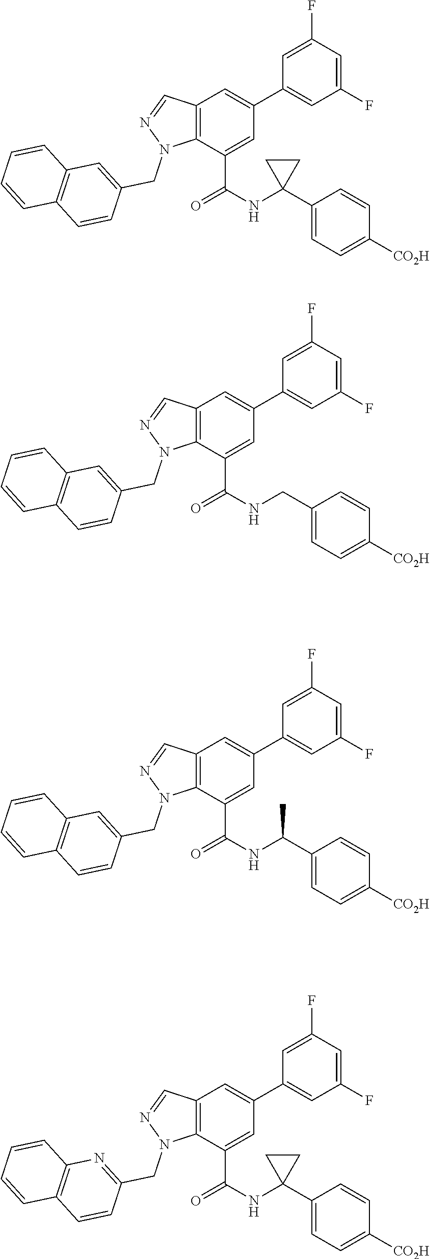 [SMILES: O=C(O)c1ccc(C2(NC(=O)c3cc(-c4cc(F)cc(F)c4)cc4cnn(Cc5ccc6ccccc6c5)c34)CC2)cc1 ; O=C(O)c1ccc(CNC(=O)c2cc(-c3cc(F)cc(F)c3)cc3cnn(Cc4ccc5ccccc5c4)c23)cc1 ; C[C@H](NC(=O)c1cc(-c2cc(F)cc(F)c2)cc2cnn(Cc3ccc4ccccc4c3)c12)c1ccc(C(=O)O)cc1 ; O=C(O)c1ccc(C2(NC(=O)c3cc(-c4cc(F)cc(F)c4)cc4cnn(Cc5ccc6ccccc6n5)c34)CC2)cc1]

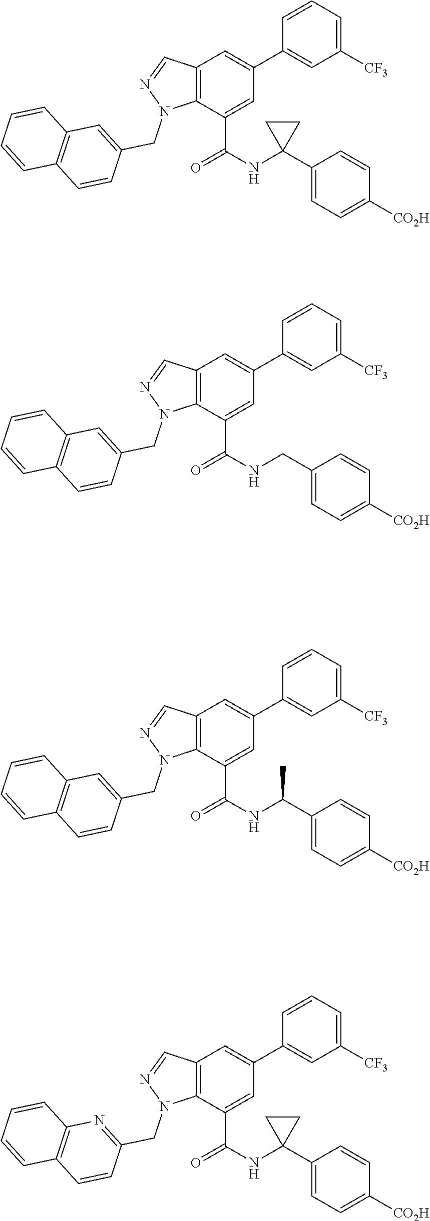 [SMILES: O=C(O)c1ccc(C2(NC(=O)c3cc(-c4cccc(C(F)(F)F)c4)cc4cnn(Cc5ccc6ccccc6c5)c34)CC2)cc1 ; O=C(O)c1ccc(CNC(=O)c2cc(-c3cccc(C(F)(F)F)c3)cc3cnn(Cc4ccc5ccccc5c4)c23)cc1 ; C[C@H](NC(=O)c1cc(-c2cccc(C(F)(F)F)c2)cc2cnn(Cc3ccc4ccccc4c3)c12)c1ccc(C(=O)O)cc1 ; O=C(O)c1ccc(C2(NC(=O)c3cc(-c4cccc(C(F)(F)F)c4)cc4cnn(Cc5ccc6ccccc6n5)c34)CC2)cc1]

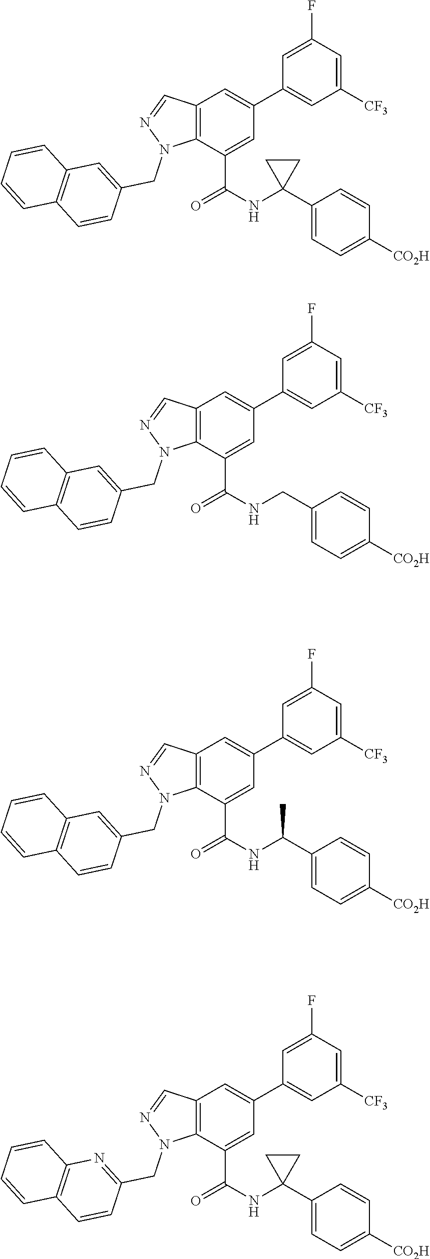 [SMILES: O=C(O)c1ccc(C2(NC(=O)c3cc(-c4cc(F)cc(C(F)(F)F)c4)cc4cnn(Cc5ccc6ccccc6c5)c34)CC2)cc1 ; O=C(O)c1ccc(CNC(=O)c2cc(-c3cc(F)cc(C(F)(F)F)c3)cc3cnn(Cc4ccc5ccccc5c4)c23)cc1 ; C[C@H](NC(=O)c1cc(-c2cc(F)cc(C(F)(F)F)c2)cc2cnn(Cc3ccc4ccccc4c3)c12)c1ccc(C(=O)O)cc1 ; O=C(O)c1ccc(C2(NC(=O)c3cc(-c4cc(F)cc(C(F)(F)F)c4)cc4cnn(Cc5ccc6ccccc6n5)c34)CC2)cc1]

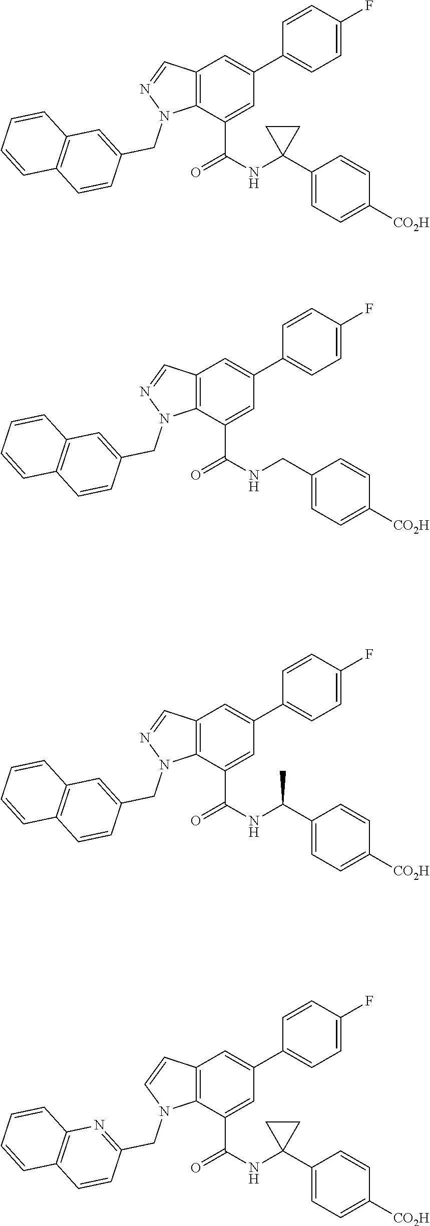 [SMILES: O=C(O)c1ccc(C2(NC(=O)c3cc(-c4ccc(F)cc4)cc4cnn(Cc5ccc6ccccc6c5)c34)CC2)cc1 ; O=C(O)c1ccc(CNC(=O)c2cc(-c3ccc(F)cc3)cc3cnn(Cc4ccc5ccccc5c4)c23)cc1 ; C[C@H](NC(=O)c1cc(-c2ccc(F)cc2)cc2cnn(Cc3ccc4ccccc4c3)c12)c1ccc(C(=O)O)cc1 ; O=C(O)c1ccc(C2(NC(=O)c3cc(-c4ccc(F)cc4)cc4ccn(Cc5ccc6ccccc6n5)c34)CC2)cc1]

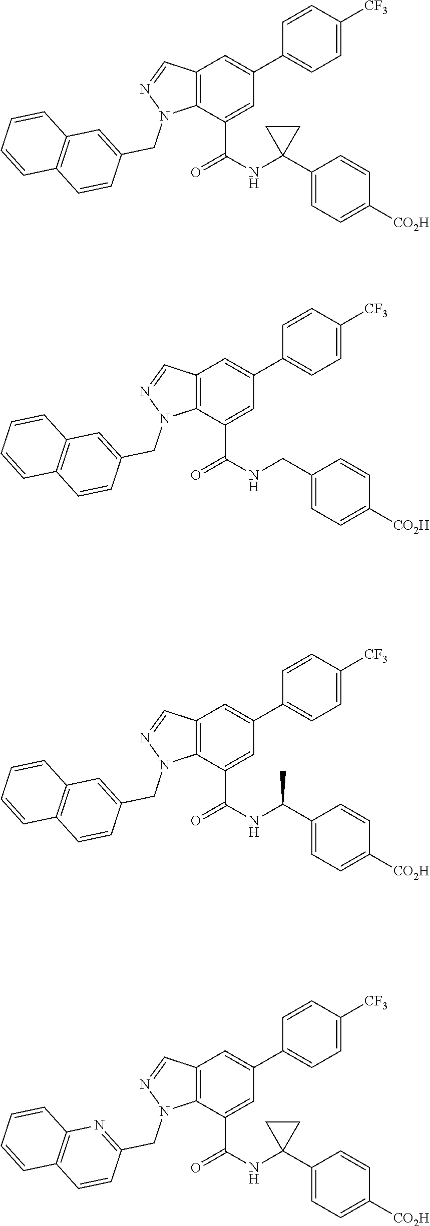 [SMILES: O=C(O)c1ccc(C2(NC(=O)c3cc(-c4ccc(C(F)(F)F)cc4)cc4cnn(Cc5ccc6ccccc6c5)c34)CC2)cc1 ; O=C(O)c1ccc(CNC(=O)c2cc(-c3ccc(C(F)(F)F)cc3)cc3cnn(Cc4ccc5ccccc5c4)c23)cc1 ; C[C@H](NC(=O)c1cc(-c2ccc(C(F)(F)F)cc2)cc2cnn(Cc3ccc4ccccc4c3)c12)c1ccc(C(=O)O)cc1 ; O=C(O)c1ccc(C2(NC(=O)c3cc(-c4ccc(C(F)(F)F)cc4)cc4cnn(Cc5ccc6ccccc6n5)c34)CC2)cc1]

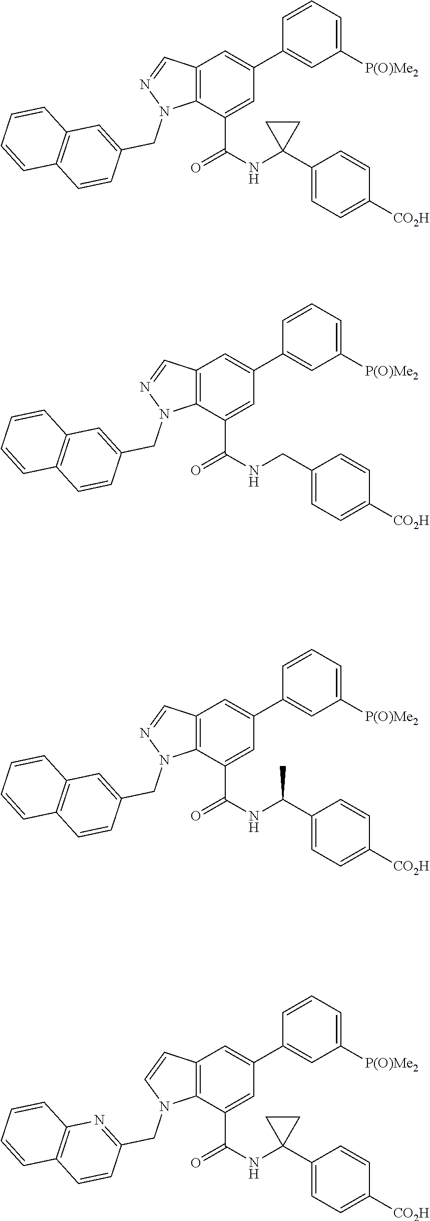

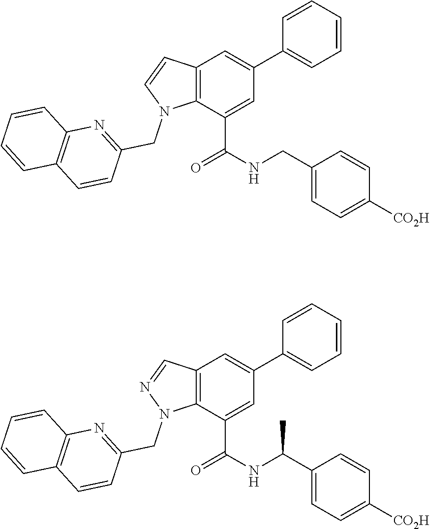 [SMILES: O=C(O)c1ccc(CNC(=O)c2cc(-c3ccccc3)cc3ccn(Cc4ccc5ccccc5n4)c23)cc1 ; C[C@H](NC(=O)c1cc(-c2ccccc2)cc2cnn(Cc3ccc4ccccc4n3)c12)c1ccc(C(=O)O)cc1]

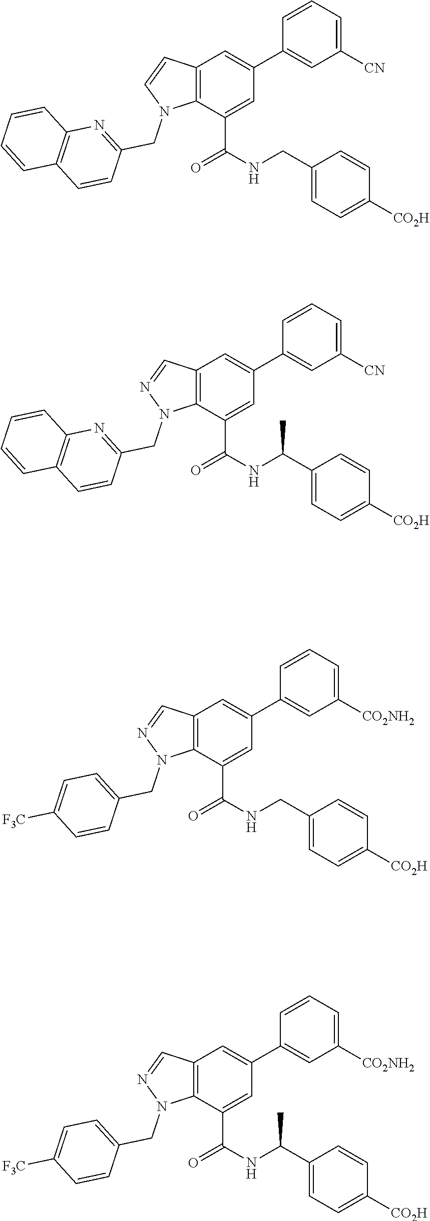

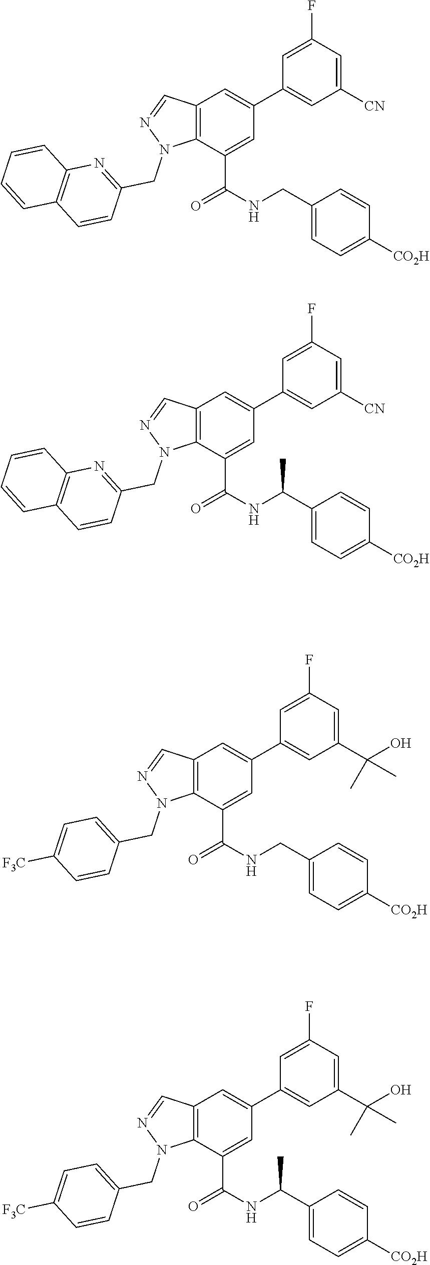 [SMILES: N#Cc1cc(F)cc(-c2cc(C(=O)NCc3ccc(C(=O)O)cc3)c3c(cnn3Cc3ccc4ccccc4n3)c2)c1 ; C[C@H](NC(=O)c1cc(-c2cc(F)cc(C#N)c2)cc2cnn(Cc3ccc4ccccc4n3)c12)c1ccc(C(=O)O)cc1 ; CC(C)(O)c1cc(F)cc(-c2cc(C(=O)NCc3ccc(C(=O)O)cc3)c3c(cnn3Cc3ccc(C(F)(F)F)cc3)c2)c1 ; C[C@H](NC(=O)c1cc(-c2cc(F)cc(C(C)(C)O)c2)cc2cnn(Cc3ccc(C(F)(F)F)cc3)c12)c1ccc(C(=O)O)cc1]

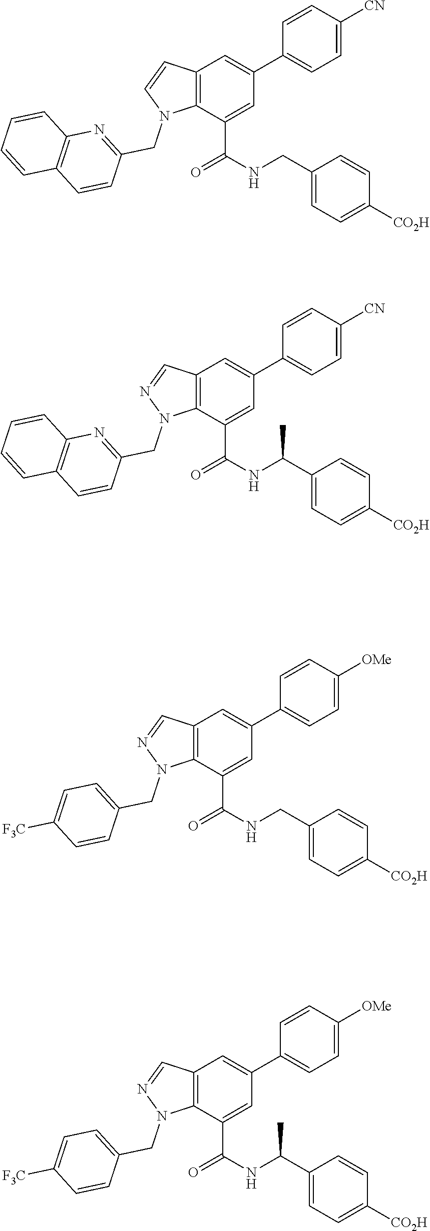 [SMILES: N#Cc1ccc(-c2cc(C(=O)NCc3ccc(C(=O)O)cc3)c3c(ccn3Cc3ccc4ccccc4n3)c2)cc1 ; C[C@H](NC(=O)c1cc(-c2ccc(C#N)cc2)cc2cnn(Cc3ccc4ccccc4n3)c12)c1ccc(C(=O)O)cc1 ; COc1ccc(-c2cc(C(=O)NCc3ccc(C(=O)O)cc3)c3c(cnn3Cc3ccc(C(F)(F)F)cc3)c2)cc1 ; COc1ccc(-c2cc(C(=O)N[C@@H](C)c3ccc(C(=O)O)cc3)c3c(cnn3Cc3ccc(C(F)(F)F)cc3)c2)cc1]

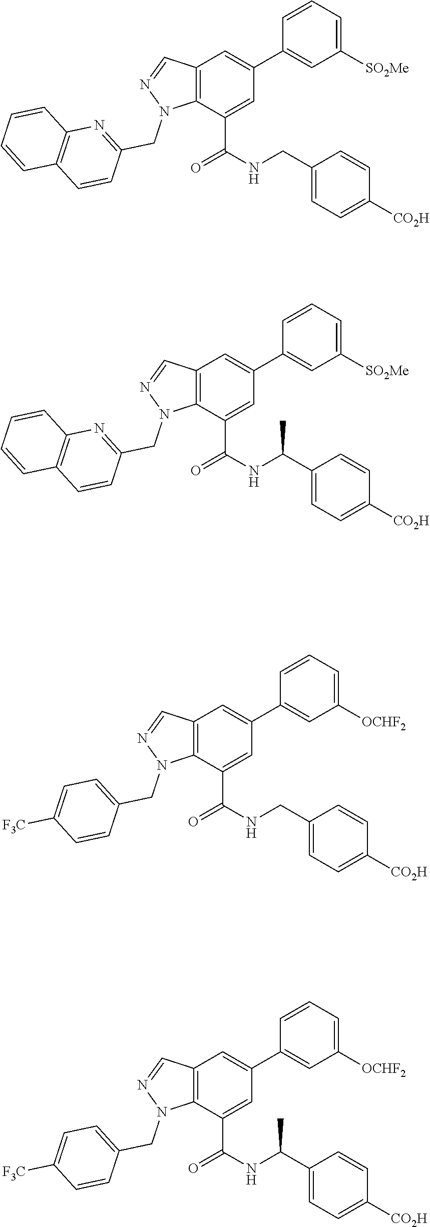 [SMILES: CS(=O)(=O)c1cccc(-c2cc(C(=O)NCc3ccc(C(=O)O)cc3)c3c(cnn3Cc3ccc4ccccc4n3)c2)c1 ; C[C@H](NC(=O)c1cc(-c2cccc(S(C)(=O)=O)c2)cc2cnn(Cc3ccc4ccccc4n3)c12)c1ccc(C(=O)O)cc1 ; O=C(O)c1ccc(CNC(=O)c2cc(-c3cccc(OC(F)F)c3)cc3cnn(Cc4ccc(C(F)(F)F)cc4)c23)cc1 ; C[C@H](NC(=O)c1cc(-c2cccc(OC(F)F)c2)cc2cnn(Cc3ccc(C(F)(F)F)cc3)c12)c1ccc(C(=O)O)cc1]

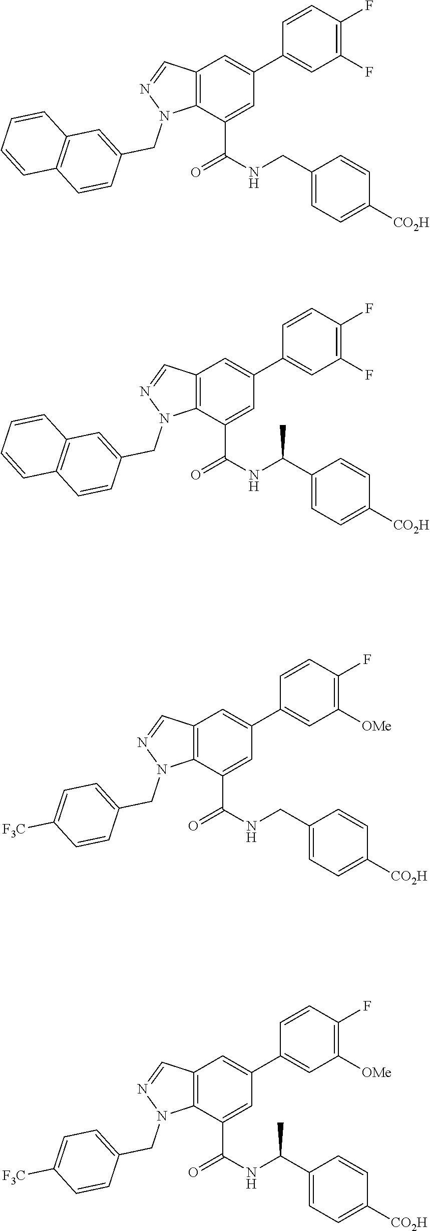 [SMILES: O=C(O)c1ccc(CNC(=O)c2cc(-c3ccc(F)c(F)c3)cc3cnn(Cc4ccc5ccccc5c4)c23)cc1 ; C[C@H](NC(=O)c1cc(-c2ccc(F)c(F)c2)cc2cnn(Cc3ccc4ccccc4c3)c12)c1ccc(C(=O)O)cc1 ; COc1cc(-c2cc(C(=O)NCc3ccc(C(=O)O)cc3)c3c(cnn3Cc3ccc(C(F)(F)F)cc3)c2)ccc1F ; COc1cc(-c2cc(C(=O)N[C@@H](C)c3ccc(C(=O)O)cc3)c3c(cnn3Cc3ccc(C(F)(F)F)cc3)c2)ccc1F]

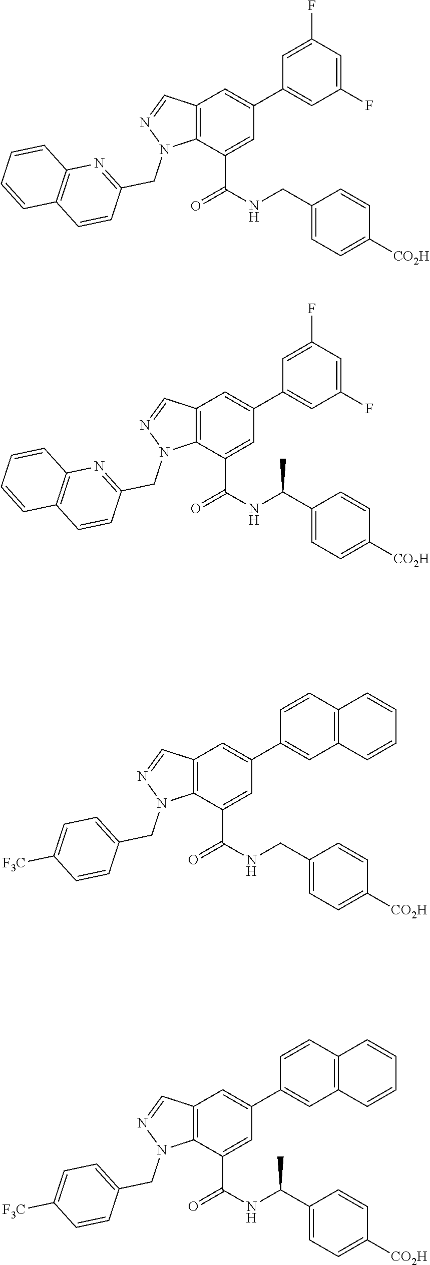 [SMILES: O=C(O)c1ccc(CNC(=O)c2cc(-c3cc(F)cc(F)c3)cc3cnn(Cc4ccc5ccccc5n4)c23)cc1 ; C[C@H](NC(=O)c1cc(-c2cc(F)cc(F)c2)cc2cnn(Cc3ccc4ccccc4n3)c12)c1ccc(C(=O)O)cc1 ; O=C(O)c1ccc(CNC(=O)c2cc(-c3ccc4ccccc4c3)cc3cnn(Cc4ccc(C(F)(F)F)cc4)c23)cc1 ; C[C@H](NC(=O)c1cc(-c2ccc3ccccc3c2)cc2cnn(Cc3ccc(C(F)(F)F)cc3)c12)c1ccc(C(=O)O)cc1]

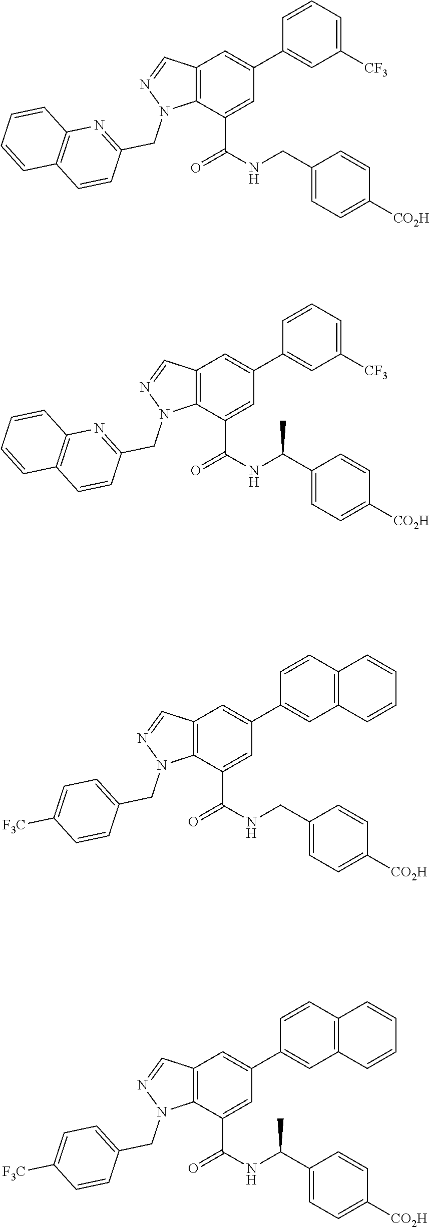 [SMILES: O=C(O)c1ccc(CNC(=O)c2cc(-c3cccc(C(F)(F)F)c3)cc3cnn(Cc4ccc5ccccc5n4)c23)cc1 ; C[C@H](NC(=O)c1cc(-c2cccc(C(F)(F)F)c2)cc2cnn(Cc3ccc4ccccc4n3)c12)c1ccc(C(=O)O)cc1 ; O=C(O)c1ccc(CNC(=O)c2cc(-c3ccc4ccccc4c3)cc3cnn(Cc4ccc(C(F)(F)F)cc4)c23)cc1 ; C[C@H](NC(=O)c1cc(-c2ccc3ccccc3c2)cc2cnn(Cc3ccc(C(F)(F)F)cc3)c12)c1ccc(C(=O)O)cc1]

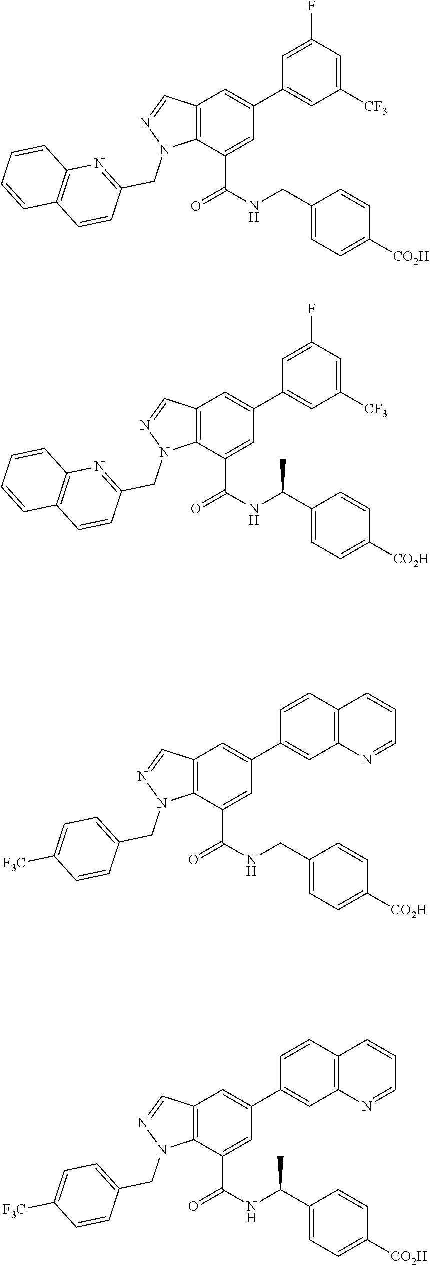 [SMILES: O=C(O)c1ccc(CNC(=O)c2cc(-c3cc(F)cc(C(F)(F)F)c3)cc3cnn(Cc4ccc5ccccc5n4)c23)cc1 ; C[C@H](NC(=O)c1cc(-c2cc(F)cc(C(F)(F)F)c2)cc2cnn(Cc3ccc4ccccc4n3)c12)c1ccc(C(=O)O)cc1 ; O=C(O)c1ccc(CNC(=O)c2cc(-c3ccc4cccnc4c3)cc3cnn(Cc4ccc(C(F)(F)F)cc4)c23)cc1 ; C[C@H](NC(=O)c1cc(-c2ccc3cccnc3c2)cc2cnn(Cc3ccc(C(F)(F)F)cc3)c12)c1ccc(C(=O)O)cc1]

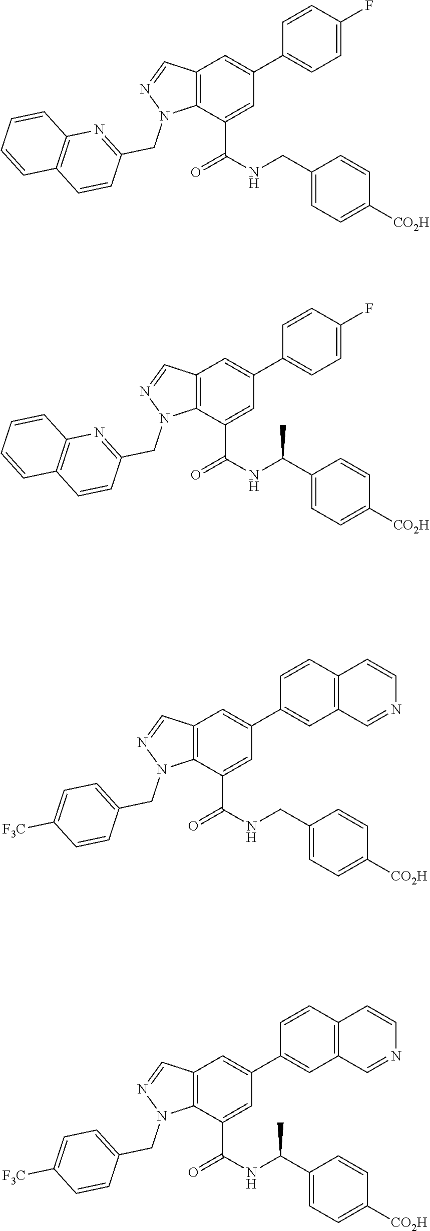 [SMILES: O=C(O)c1ccc(CNC(=O)c2cc(-c3ccc(F)cc3)cc3cnn(Cc4ccc5ccccc5n4)c23)cc1 ; C[C@H](NC(=O)c1cc(-c2ccc(F)cc2)cc2cnn(Cc3ccc4ccccc4n3)c12)c1ccc(C(=O)O)cc1 ; O=C(O)c1ccc(CNC(=O)c2cc(-c3ccc4ccncc4c3)cc3cnn(Cc4ccc(C(F)(F)F)cc4)c23)cc1 ; C[C@H](NC(=O)c1cc(-c2ccc3ccncc3c2)cc2cnn(Cc3ccc(C(F)(F)F)cc3)c12)c1ccc(C(=O)O)cc1]

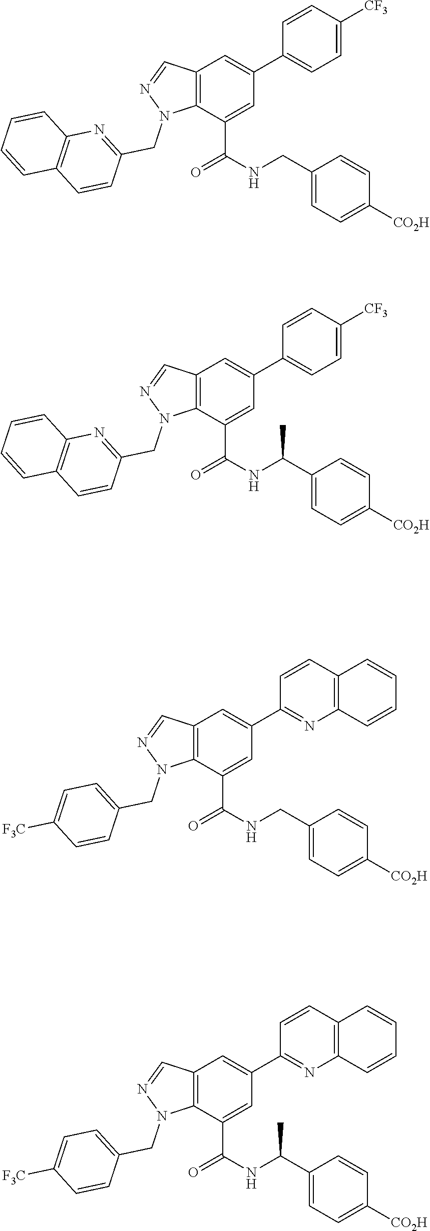 [SMILES: O=C(O)c1ccc(CNC(=O)c2cc(-c3ccc(C(F)(F)F)cc3)cc3cnn(Cc4ccc5ccccc5n4)c23)cc1 ; C[C@H](NC(=O)c1cc(-c2ccc(C(F)(F)F)cc2)cc2cnn(Cc3ccc4ccccc4n3)c12)c1ccc(C(=O)O)cc1 ; O=C(O)c1ccc(CNC(=O)c2cc(-c3ccc4ccccc4n3)cc3cnn(Cc4ccc(C(F)(F)F)cc4)c23)cc1 ; C[C@H](NC(=O)c1cc(-c2ccc3ccccc3n2)cc2cnn(Cc3ccc(C(F)(F)F)cc3)c12)c1ccc(C(=O)O)cc1]

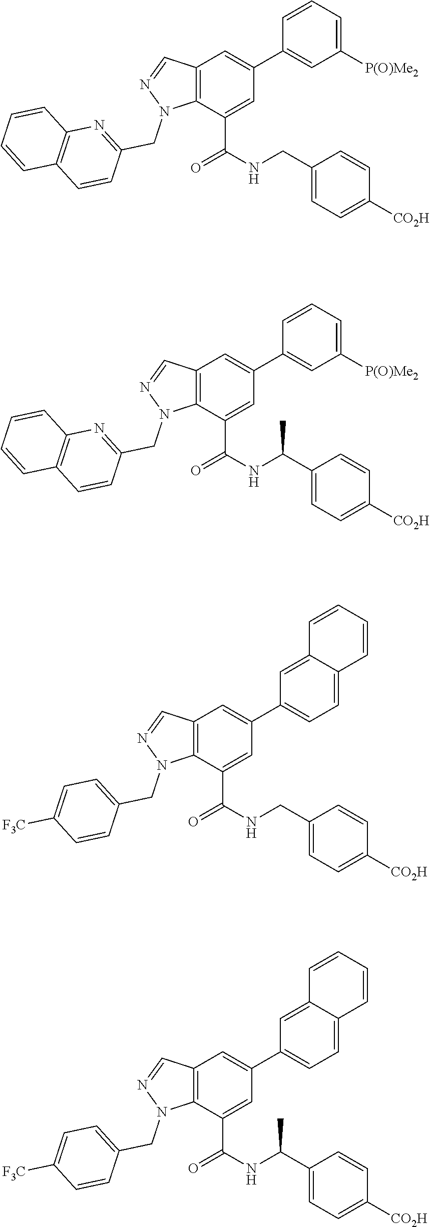

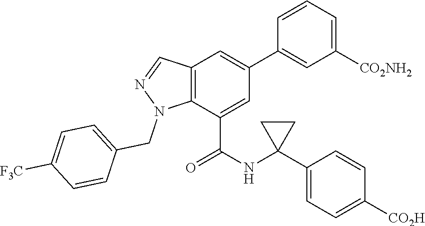

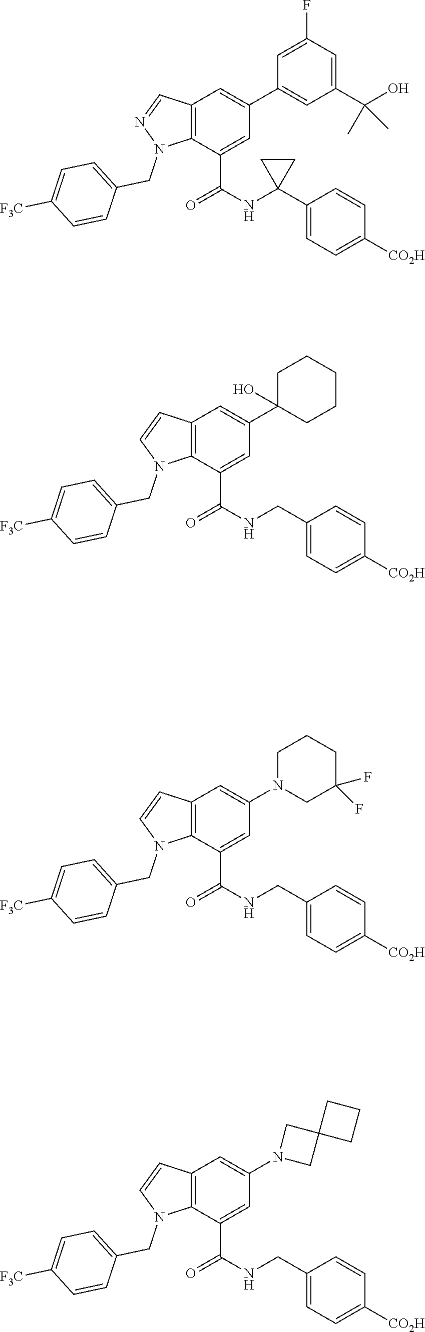 [SMILES: CC(C)(O)c1cc(F)cc(-c2cc(C(=O)NC3(c4ccc(C(=O)O)cc4)CC3)c3c(cnn3Cc3ccc(C(F)(F)F)cc3)c2)c1 ; O=C(O)c1ccc(CNC(=O)c2cc(C3(O)CCCCC3)cc3ccn(Cc4ccc(C(F)(F)F)cc4)c23)cc1 ; O=C(O)c1ccc(CNC(=O)c2cc(N3CCCC(F)(F)C3)cc3ccn(Cc4ccc(C(F)(F)F)cc4)c23)cc1 ; O=C(O)c1ccc(CNC(=O)c2cc(N3CC4(CCC4)C3)cc3ccn(Cc4ccc(C(F)(F)F)cc4)c23)cc1]

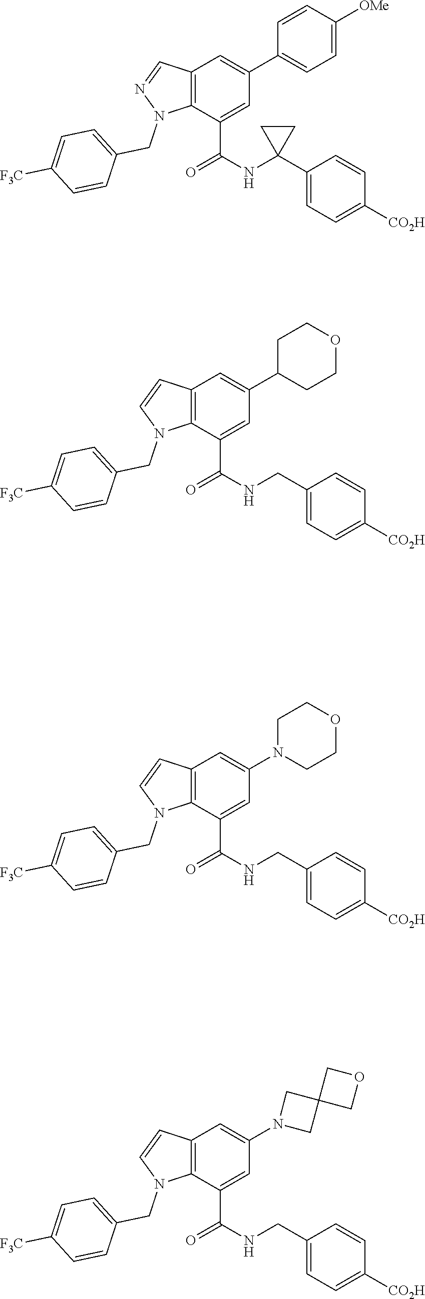 [SMILES: COc1ccc(-c2cc(C(=O)NC3(c4ccc(C(=O)O)cc4)CC3)c3c(cnn3Cc3ccc(C(F)(F)F)cc3)c2)cc1 ; O=C(O)c1ccc(CNC(=O)c2cc(C3CCOCC3)cc3ccn(Cc4ccc(C(F)(F)F)cc4)c23)cc1 ; O=C(O)c1ccc(CNC(=O)c2cc(N3CCOCC3)cc3ccn(Cc4ccc(C(F)(F)F)cc4)c23)cc1 ; O=C(O)c1ccc(CNC(=O)c2cc(N3CC4(COC4)C3)cc3ccn(Cc4ccc(C(F)(F)F)cc4)c23)cc1]

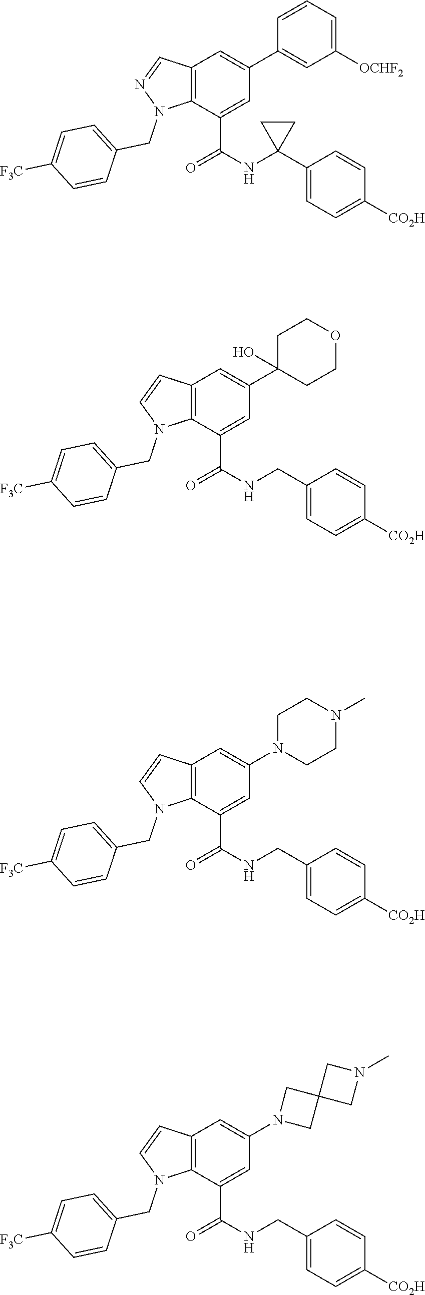 [SMILES: O=C(O)c1ccc(C2(NC(=O)c3cc(-c4cccc(OC(F)F)c4)cc4cnn(Cc5ccc(C(F)(F)F)cc5)c34)CC2)cc1 ; O=C(O)c1ccc(CNC(=O)c2cc(C3(O)CCOCC3)cc3ccn(Cc4ccc(C(F)(F)F)cc4)c23)cc1 ; CN1CCN(c2cc(C(=O)NCc3ccc(C(=O)O)cc3)c3c(ccn3Cc3ccc(C(F)(F)F)cc3)c2)CC1 ; CN1CC2(C1)CN(c1cc(C(=O)NCc3ccc(C(=O)O)cc3)c3c(ccn3Cc3ccc(C(F)(F)F)cc3)c1)C2]

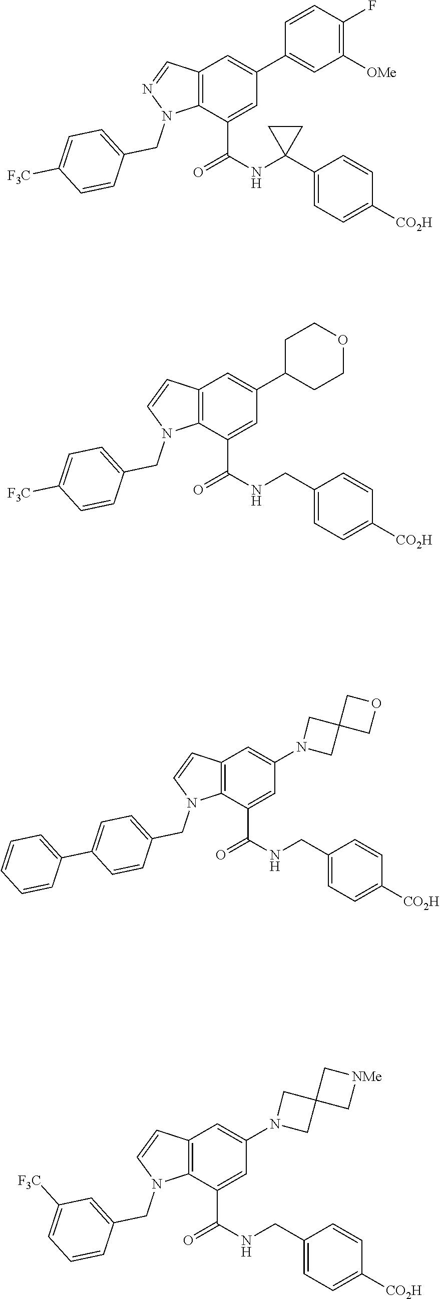 [SMILES: COc1cc(-c2cc(C(=O)NC3(c4ccc(C(=O)O)cc4)CC3)c3c(cnn3Cc3ccc(C(F)(F)F)cc3)c2)ccc1F ; O=C(O)c1ccc(CNC(=O)c2cc(C3CCOCC3)cc3ccn(Cc4ccc(C(F)(F)F)cc4)c23)cc1 ; O=C(O)c1ccc(CNC(=O)c2cc(N3CC4(COC4)C3)cc3ccn(Cc4ccc(-c5ccccc5)cc4)c23)cc1 ; CN1CC2(C1)CN(c1cc(C(=O)NCc3ccc(C(=O)O)cc3)c3c(ccn3Cc3cccc(C(F)(F)F)c3)c1)C2]

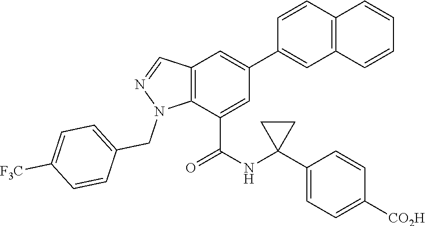 [SMILES: O=C(O)c1ccc(C2(NC(=O)c3cc(-c4ccc5ccccc5c4)cc4cnn(Cc5ccc(C(F)(F)F)cc5)c34)CC2)cc1]

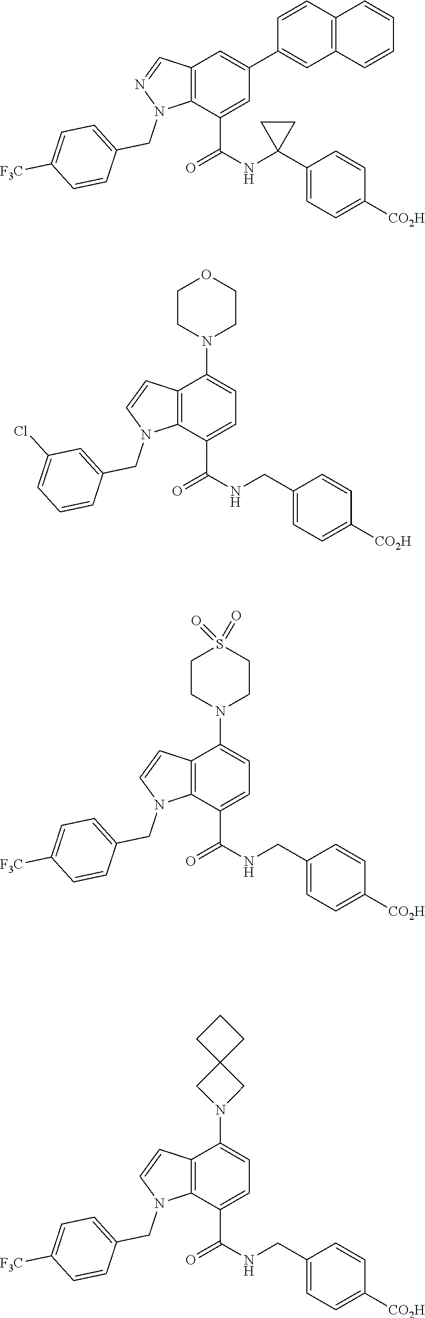 [SMILES: O=C(O)c1ccc(C2(NC(=O)c3cc(-c4ccc5ccccc5c4)cc4cnn(Cc5ccc(C(F)(F)F)cc5)c34)CC2)cc1 ; O=C(O)c1ccc(CNC(=O)c2ccc(N3CCOCC3)c3ccn(Cc4cccc(Cl)c4)c23)cc1 ; O=C(O)c1ccc(CNC(=O)c2ccc(N3CCS(=O)(=O)CC3)c3ccn(Cc4ccc(C(F)(F)F)cc4)c23)cc1 ; O=C(O)c1ccc(CNC(=O)c2ccc(N3CC4(CCC4)C3)c3ccn(Cc4ccc(C(F)(F)F)cc4)c23)cc1]

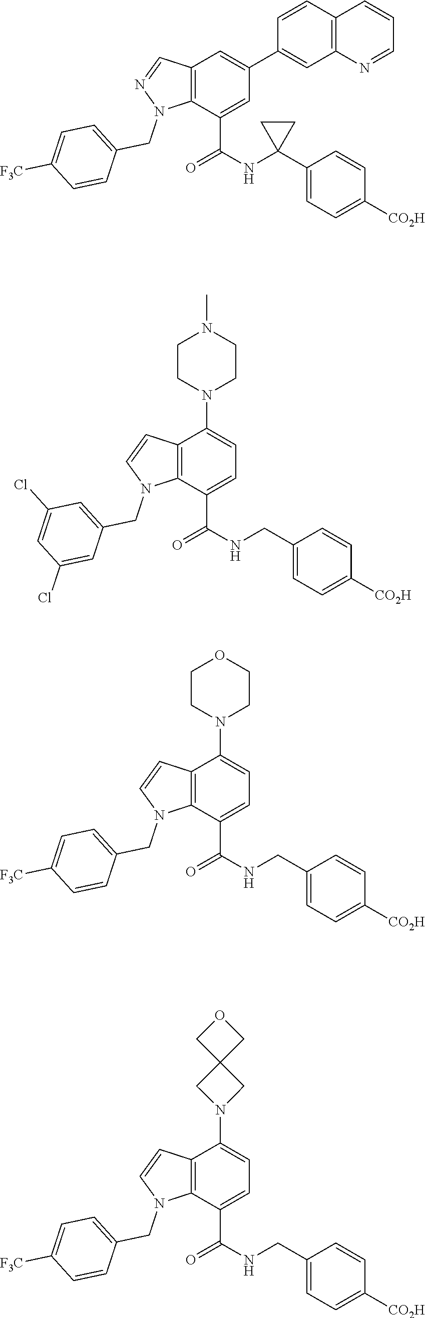 [SMILES: O=C(O)c1ccc(C2(NC(=O)c3cc(-c4ccc5cccnc5c4)cc4cnn(Cc5ccc(C(F)(F)F)cc5)c34)CC2)cc1 ; CN1CCN(c2ccc(C(=O)NCc3ccc(C(=O)O)cc3)c3c2ccn3Cc2cc(Cl)cc(Cl)c2)CC1 ; O=C(O)c1ccc(CNC(=O)c2ccc(N3CCOCC3)c3ccn(Cc4ccc(C(F)(F)F)cc4)c23)cc1 ; O=C(O)c1ccc(CNC(=O)c2ccc(N3CC4(COC4)C3)c3ccn(Cc4ccc(C(F)(F)F)cc4)c23)cc1]

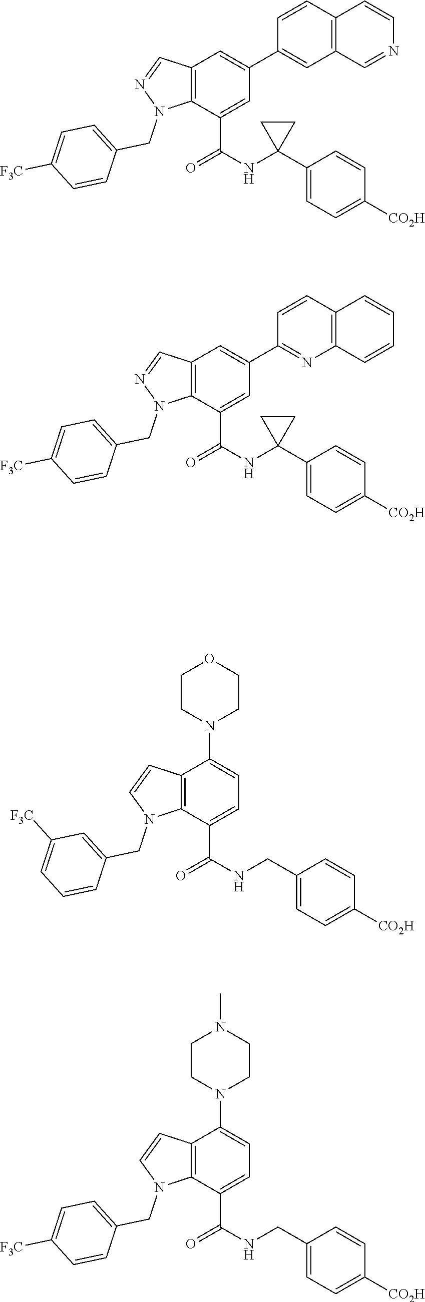 [SMILES: O=C(O)c1ccc(C2(NC(=O)c3cc(-c4ccc5ccncc5c4)cc4cnn(Cc5ccc(C(F)(F)F)cc5)c34)CC2)cc1 ; O=C(O)c1ccc(C2(NC(=O)c3cc(-c4ccc5ccccc5n4)cc4cnn(Cc5ccc(C(F)(F)F)cc5)c34)CC2)cc1 ; O=C(O)c1ccc(CNC(=O)c2ccc(N3CCOCC3)c3ccn(Cc4cccc(C(F)(F)F)c4)c23)cc1 ; CN1CCN(c2ccc(C(=O)NCc3ccc(C(=O)O)cc3)c3c2ccn3Cc2ccc(C(F)(F)F)cc2)CC1]

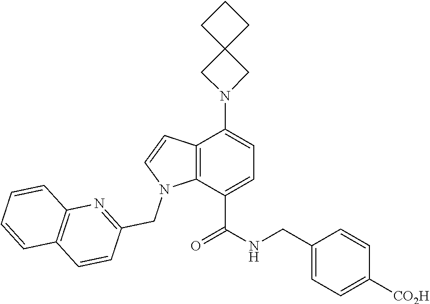 [SMILES: O=C(O)c1ccc(CNC(=O)c2ccc(N3CC4(CCC4)C3)c3ccn(Cc4ccc5ccccc5n4)c23)cc1]

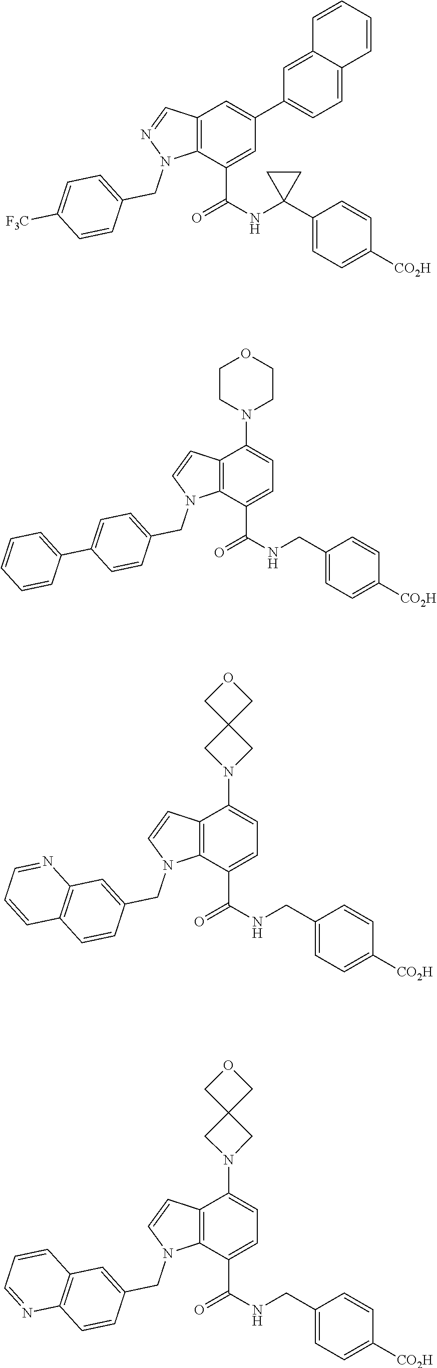 [SMILES: O=C(O)c1ccc(C2(NC(=O)c3cc(-c4ccc5ccccc5c4)cc4cnn(Cc5ccc(C(F)(F)F)cc5)c34)CC2)cc1 ; O=C(O)c1ccc(CNC(=O)c2ccc(N3CCOCC3)c3ccn(Cc4ccc(-c5ccccc5)cc4)c23)cc1 ; O=C(O)c1ccc(CNC(=O)c2ccc(N3CC4(COC4)C3)c3ccn(Cc4ccc5cccnc5c4)c23)cc1 ; O=C(O)c1ccc(CNC(=O)c2ccc(N3CC4(COC4)C3)c3ccn(Cc4ccc5ncccc5c4)c23)cc1]

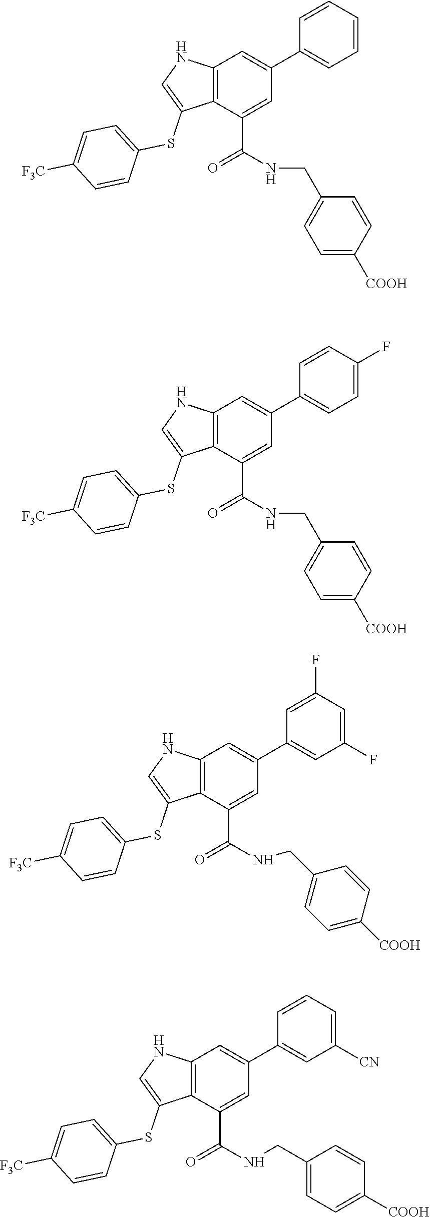 [SMILES: O=C(O)c1ccc(CNC(=O)c2cc(-c3ccccc3)cc3[nH]cc(Sc4ccc(C(F)(F)F)cc4)c23)cc1 ; O=C(O)c1ccc(CNC(=O)c2cc(-c3ccc(F)cc3)cc3[nH]cc(Sc4ccc(C(F)(F)F)cc4)c23)cc1 ; O=C(O)c1ccc(CNC(=O)c2cc(-c3cc(F)cc(F)c3)cc3[nH]cc(Sc4ccc(C(F)(F)F)cc4)c23)cc1 ; N#Cc1cccc(-c2cc(C(=O)NCc3ccc(C(=O)O)cc3)c3c(Sc4ccc(C(F)(F)F)cc4)c[nH]c3c2)c1]

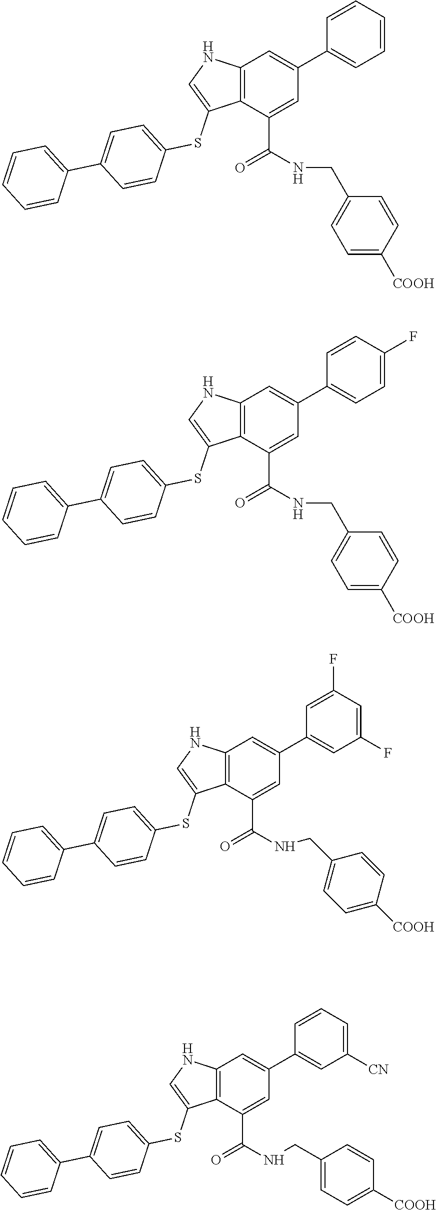 [SMILES: O=C(O)c1ccc(CNC(=O)c2cc(-c3ccccc3)cc3[nH]cc(Sc4ccc(-c5ccccc5)cc4)c23)cc1 ; O=C(O)c1ccc(CNC(=O)c2cc(-c3ccc(F)cc3)cc3[nH]cc(Sc4ccc(-c5ccccc5)cc4)c23)cc1 ; O=C(O)c1ccc(CNC(=O)c2cc(-c3cc(F)cc(F)c3)cc3[nH]cc(Sc4ccc(-c5ccccc5)cc4)c23)cc1 ; N#Cc1cccc(-c2cc(C(=O)NCc3ccc(C(=O)O)cc3)c3c(Sc4ccc(-c5ccccc5)cc4)c[nH]c3c2)c1]

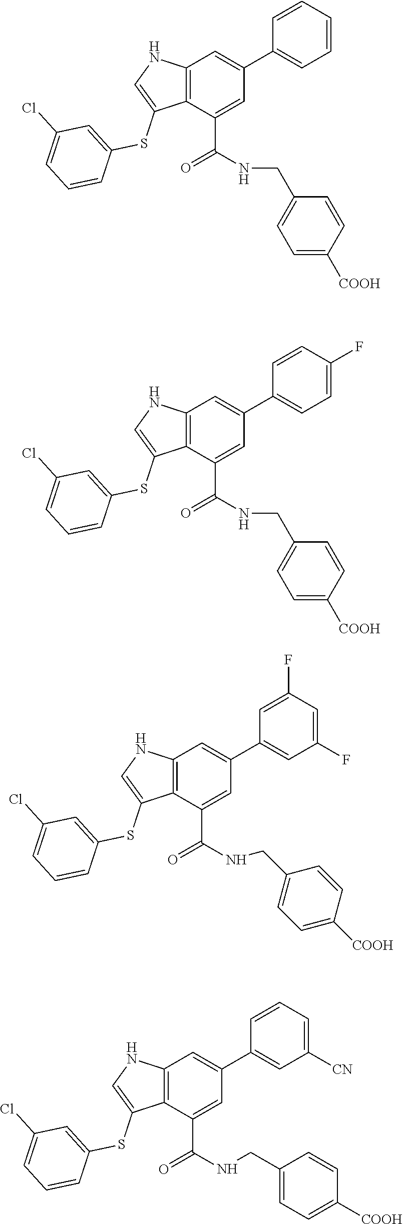 [SMILES: O=C(O)c1ccc(CNC(=O)c2cc(-c3ccccc3)cc3[nH]cc(Sc4cccc(Cl)c4)c23)cc1 ; O=C(O)c1ccc(CNC(=O)c2cc(-c3ccc(F)cc3)cc3[nH]cc(Sc4cccc(Cl)c4)c23)cc1 ; O=C(O)c1ccc(CNC(=O)c2cc(-c3cc(F)cc(F)c3)cc3[nH]cc(Sc4cccc(Cl)c4)c23)cc1 ; N#Cc1cccc(-c2cc(C(=O)NCc3ccc(C(=O)O)cc3)c3c(Sc4cccc(Cl)c4)c[nH]c3c2)c1]

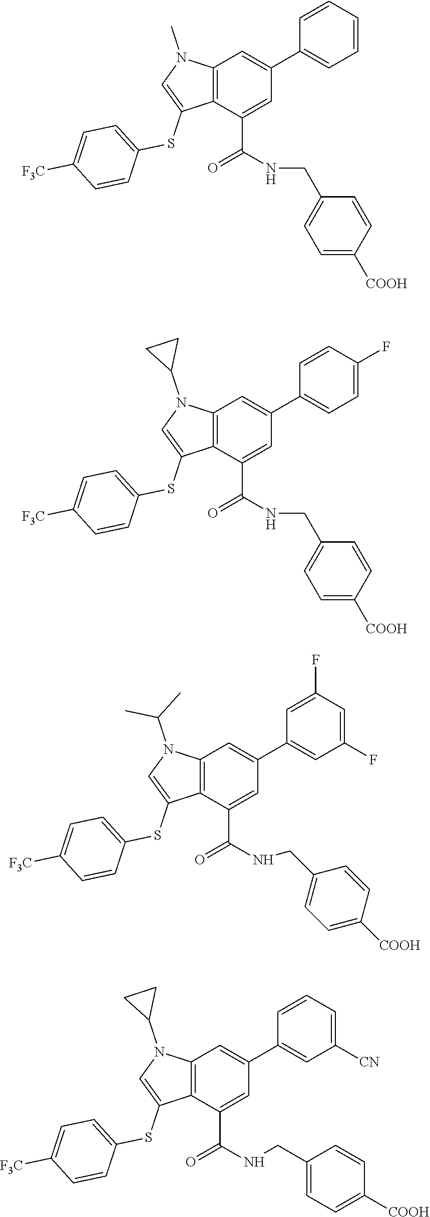 [SMILES: Cn1cc(Sc2ccc(C(F)(F)F)cc2)c2c(C(=O)NCc3ccc(C(=O)O)cc3)cc(-c3ccccc3)cc21 ; O=C(O)c1ccc(CNC(=O)c2cc(-c3ccc(F)cc3)cc3c2c(Sc2ccc(C(F)(F)F)cc2)cn3C2CC2)cc1 ; CC(C)n1cc(Sc2ccc(C(F)(F)F)cc2)c2c(C(=O)NCc3ccc(C(=O)O)cc3)cc(-c3cc(F)cc(F)c3)cc21 ; N#Cc1cccc(-c2cc(C(=O)NCc3ccc(C(=O)O)cc3)c3c(Sc4ccc(C(F)(F)F)cc4)cn(C4CC4)c3c2)c1]

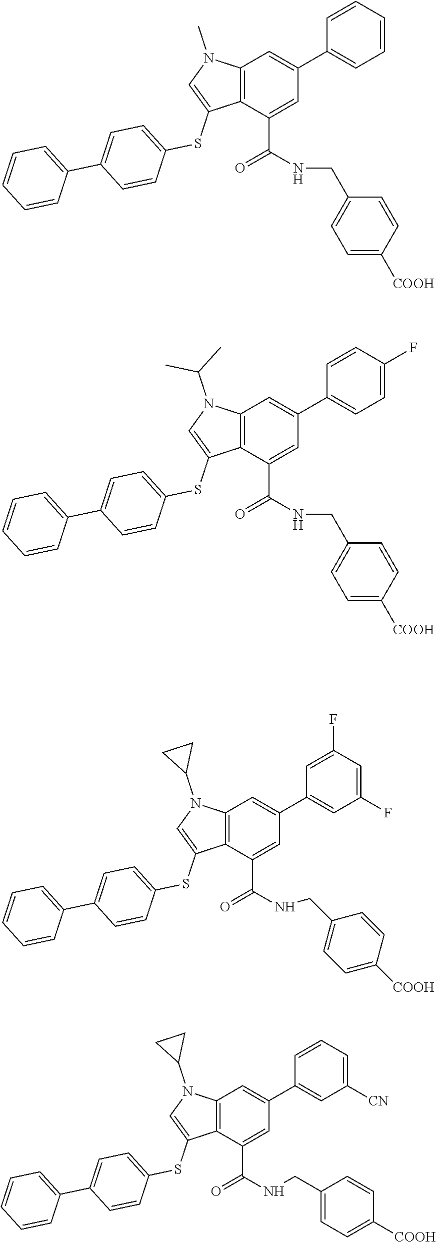 [SMILES: Cn1cc(Sc2ccc(-c3ccccc3)cc2)c2c(C(=O)NCc3ccc(C(=O)O)cc3)cc(-c3ccccc3)cc21 ; CC(C)n1cc(Sc2ccc(-c3ccccc3)cc2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Sc2ccc(-c4ccccc4)cc2)cn3C2CC2)cc1 ; N#Cc1cccc(-c2cc(C(=O)NCc3ccc(C(=O)O)cc3)c3c(Sc4ccc(-c5ccccc5)cc4)cn(C4CC4)c3c2)c1]

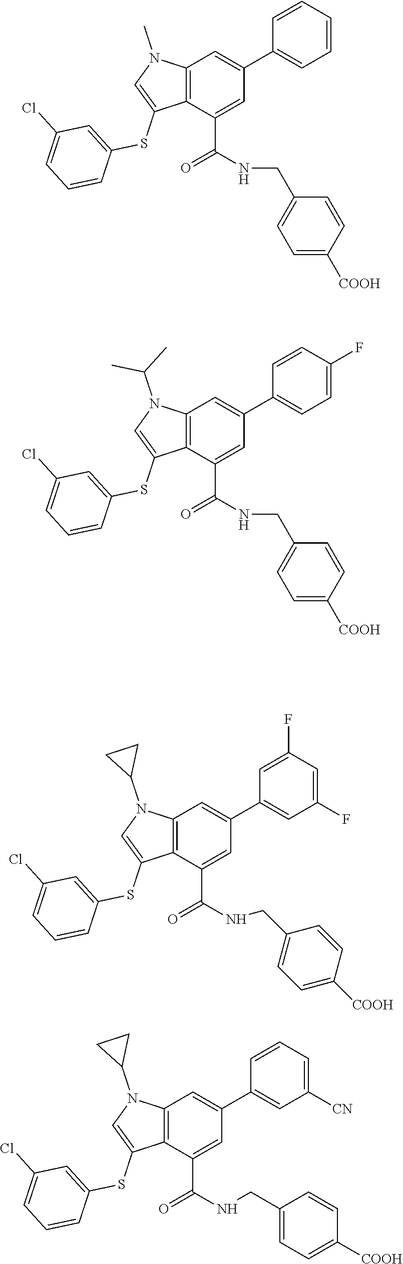 [SMILES: Cn1cc(Sc2cccc(Cl)c2)c2c(C(=O)NCc3ccc(C(=O)O)cc3)cc(-c3ccccc3)cc21 ; CC(C)n1cc(Sc2cccc(Cl)c2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Sc2cccc(Cl)c2)cn3C2CC2)cc1 ; N#Cc1cccc(-c2cc(C(=O)NCc3ccc(C(=O)O)cc3)c3c(Sc4cccc(Cl)c4)cn(C4CC4)c3c2)c1]

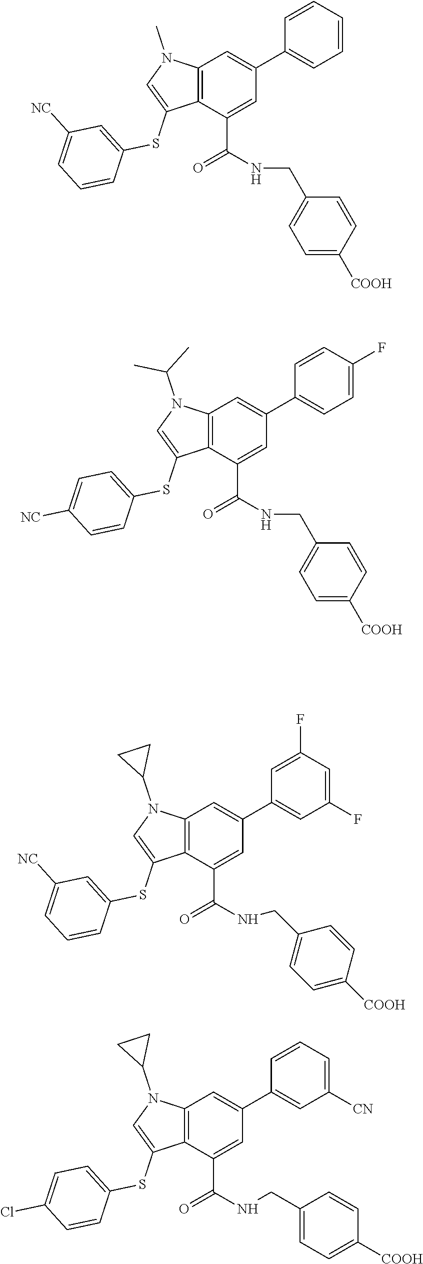 [SMILES: Cn1cc(Sc2cccc(C#N)c2)c2c(C(=O)NCc3ccc(C(=O)O)cc3)cc(-c3ccccc3)cc21 ; CC(C)n1cc(Sc2ccc(C#N)cc2)c2c(C(=O)NCc3ccc(C(=O)O)cc3)cc(-c3ccc(F)cc3)cc21 ; N#Cc1cccc(Sc2cn(C3CC3)c3cc(-c4cc(F)cc(F)c4)cc(C(=O)NCc4ccc(C(=O)O)cc4)c23)c1 ; N#Cc1cccc(-c2cc(C(=O)NCc3ccc(C(=O)O)cc3)c3c(Sc4ccc(Cl)cc4)cn(C4CC4)c3c2)c1]

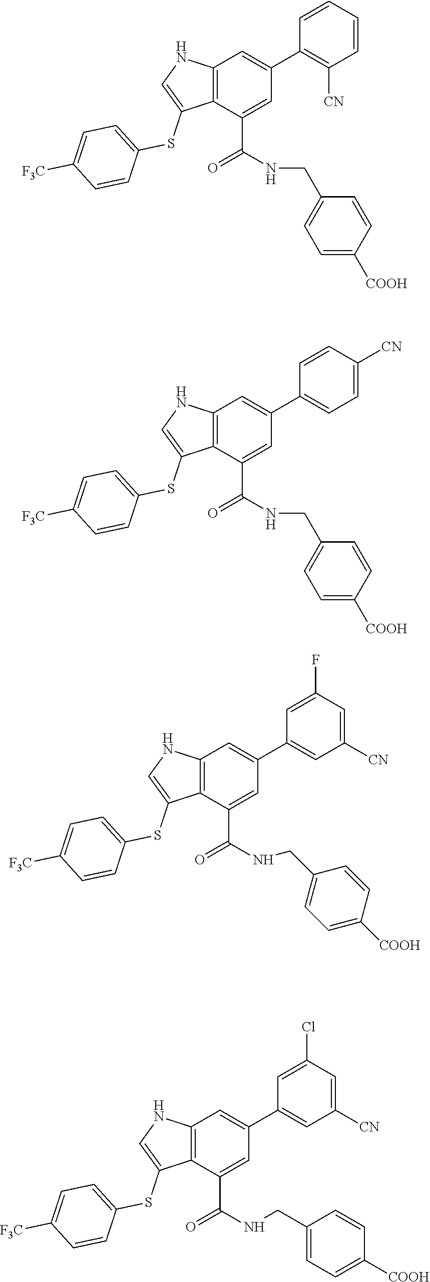 [SMILES: N#Cc1ccccc1-c1cc(C(=O)NCc2ccc(C(=O)O)cc2)c2c(Sc3ccc(C(F)(F)F)cc3)c[nH]c2c1 ; N#Cc1ccc(-c2cc(C(=O)NCc3ccc(C(=O)O)cc3)c3c(Sc4ccc(C(F)(F)F)cc4)c[nH]c3c2)cc1 ; N#Cc1cc(F)cc(-c2cc(C(=O)NCc3ccc(C(=O)O)cc3)c3c(Sc4ccc(C(F)(F)F)cc4)c[nH]c3c2)c1 ; N#Cc1cc(Cl)cc(-c2cc(C(=O)NCc3ccc(C(=O)O)cc3)c3c(Sc4ccc(C(F)(F)F)cc4)c[nH]c3c2)c1]

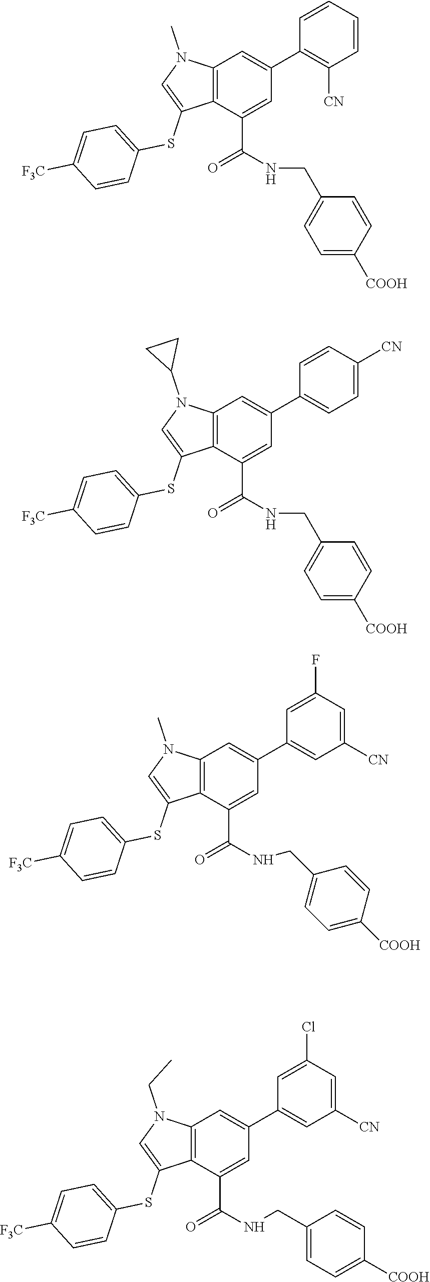 [SMILES: Cn1cc(Sc2ccc(C(F)(F)F)cc2)c2c(C(=O)NCc3ccc(C(=O)O)cc3)cc(-c3ccccc3C#N)cc21 ; N#Cc1ccc(-c2cc(C(=O)NCc3ccc(C(=O)O)cc3)c3c(Sc4ccc(C(F)(F)F)cc4)cn(C4CC4)c3c2)cc1 ; Cn1cc(Sc2ccc(C(F)(F)F)cc2)c2c(C(=O)NCc3ccc(C(=O)O)cc3)cc(-c3cc(F)cc(C#N)c3)cc21 ; CCn1cc(Sc2ccc(C(F)(F)F)cc2)c2c(C(=O)NCc3ccc(C(=O)O)cc3)cc(-c3cc(Cl)cc(C#N)c3)cc21]

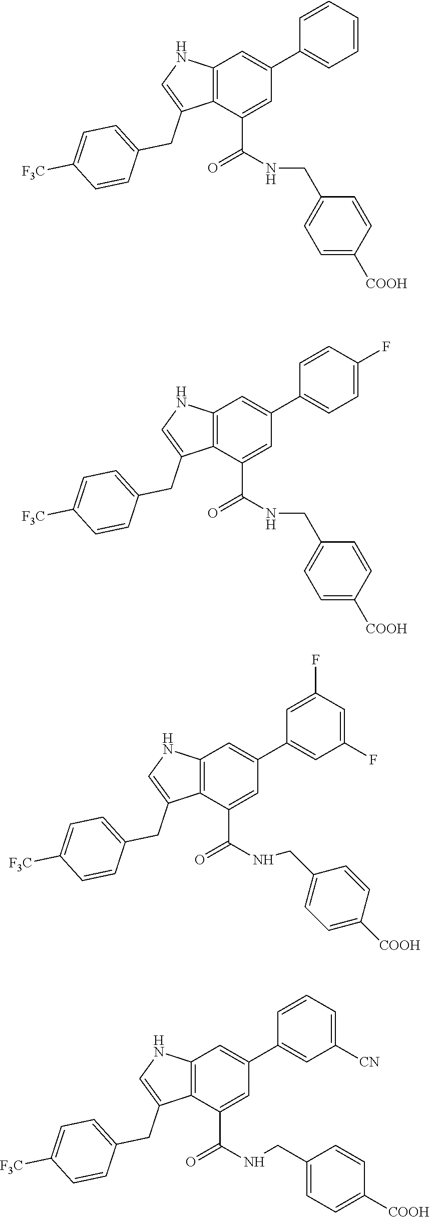 [SMILES: O=C(O)c1ccc(CNC(=O)c2cc(-c3ccccc3)cc3[nH]cc(Cc4ccc(C(F)(F)F)cc4)c23)cc1 ; O=C(O)c1ccc(CNC(=O)c2cc(-c3ccc(F)cc3)cc3[nH]cc(Cc4ccc(C(F)(F)F)cc4)c23)cc1 ; O=C(O)c1ccc(CNC(=O)c2cc(-c3cc(F)cc(F)c3)cc3[nH]cc(Cc4ccc(C(F)(F)F)cc4)c23)cc1 ; N#Cc1cccc(-c2cc(C(=O)NCc3ccc(C(=O)O)cc3)c3c(Cc4ccc(C(F)(F)F)cc4)c[nH]c3c2)c1]

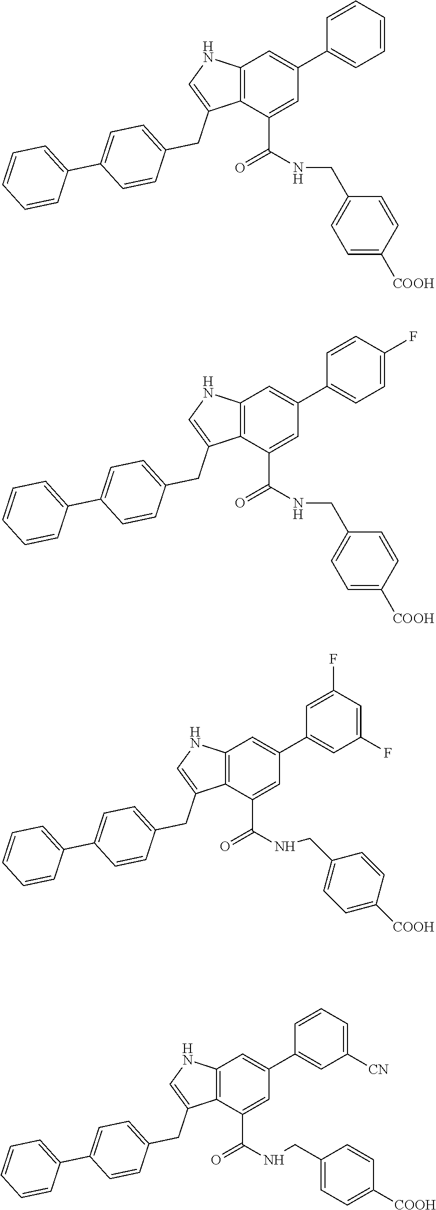 [SMILES: O=C(O)c1ccc(CNC(=O)c2cc(-c3ccccc3)cc3[nH]cc(Cc4ccc(-c5ccccc5)cc4)c23)cc1 ; O=C(O)c1ccc(CNC(=O)c2cc(-c3ccc(F)cc3)cc3[nH]cc(Cc4ccc(-c5ccccc5)cc4)c23)cc1 ; O=C(O)c1ccc(CNC(=O)c2cc(-c3cc(F)cc(F)c3)cc3[nH]cc(Cc4ccc(-c5ccccc5)cc4)c23)cc1 ; N#Cc1cccc(-c2cc(C(=O)NCc3ccc(C(=O)O)cc3)c3c(Cc4ccc(-c5ccccc5)cc4)c[nH]c3c2)c1]

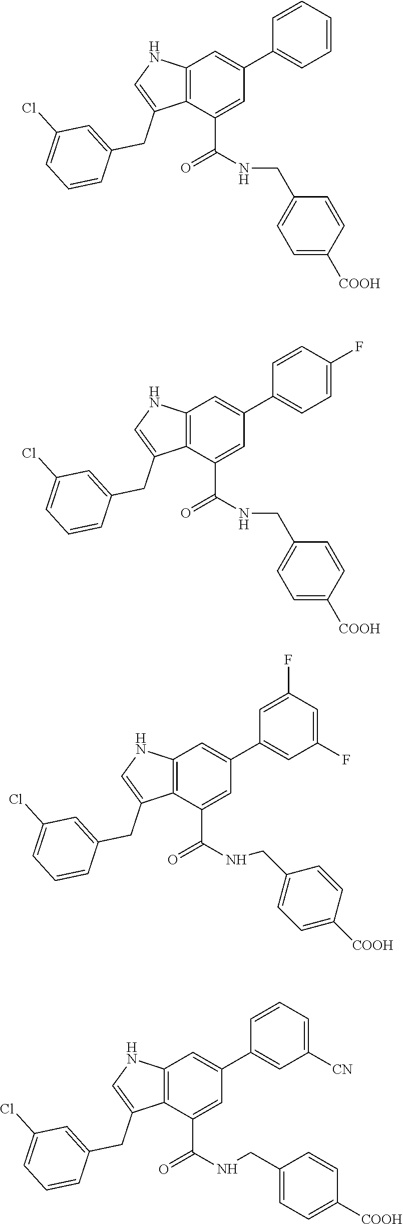 [SMILES: O=C(O)c1ccc(CNC(=O)c2cc(-c3ccccc3)cc3[nH]cc(Cc4cccc(Cl)c4)c23)cc1 ; O=C(O)c1ccc(CNC(=O)c2cc(-c3ccc(F)cc3)cc3[nH]cc(Cc4cccc(Cl)c4)c23)cc1 ; O=C(O)c1ccc(CNC(=O)c2cc(-c3cc(F)cc(F)c3)cc3[nH]cc(Cc4cccc(Cl)c4)c23)cc1 ; N#Cc1cccc(-c2cc(C(=O)NCc3ccc(C(=O)O)cc3)c3c(Cc4cccc(Cl)c4)c[nH]c3c2)c1]

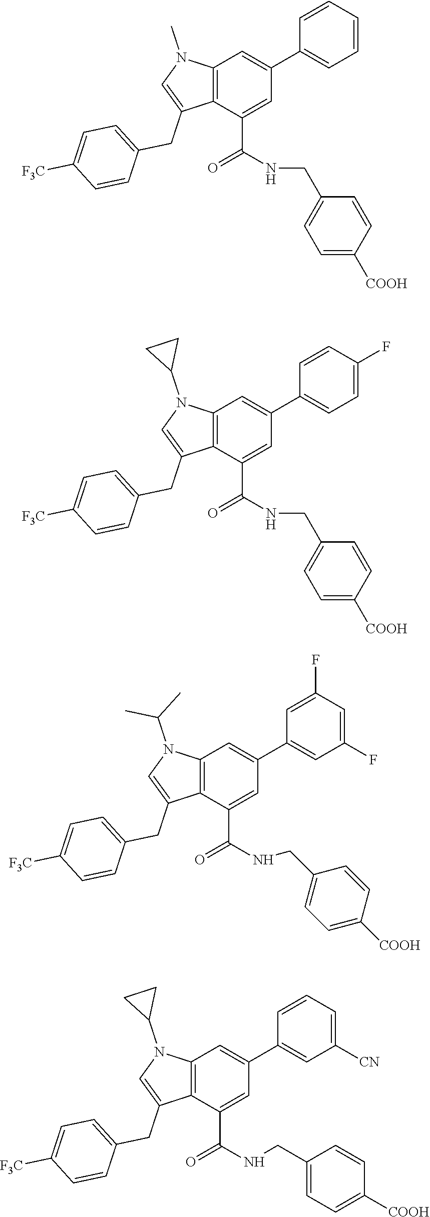 [SMILES: Cn1cc(Cc2ccc(C(F)(F)F)cc2)c2c(C(=O)NCc3ccc(C(=O)O)cc3)cc(-c3ccccc3)cc21 ; O=C(O)c1ccc(CNC(=O)c2cc(-c3ccc(F)cc3)cc3c2c(Cc2ccc(C(F)(F)F)cc2)cn3C2CC2)cc1 ; CC(C)n1cc(Cc2ccc(C(F)(F)F)cc2)c2c(C(=O)NCc3ccc(C(=O)O)cc3)cc(-c3cc(F)cc(F)c3)cc21 ; N#Cc1cccc(-c2cc(C(=O)NCc3ccc(C(=O)O)cc3)c3c(Cc4ccc(C(F)(F)F)cc4)cn(C4CC4)c3c2)c1]

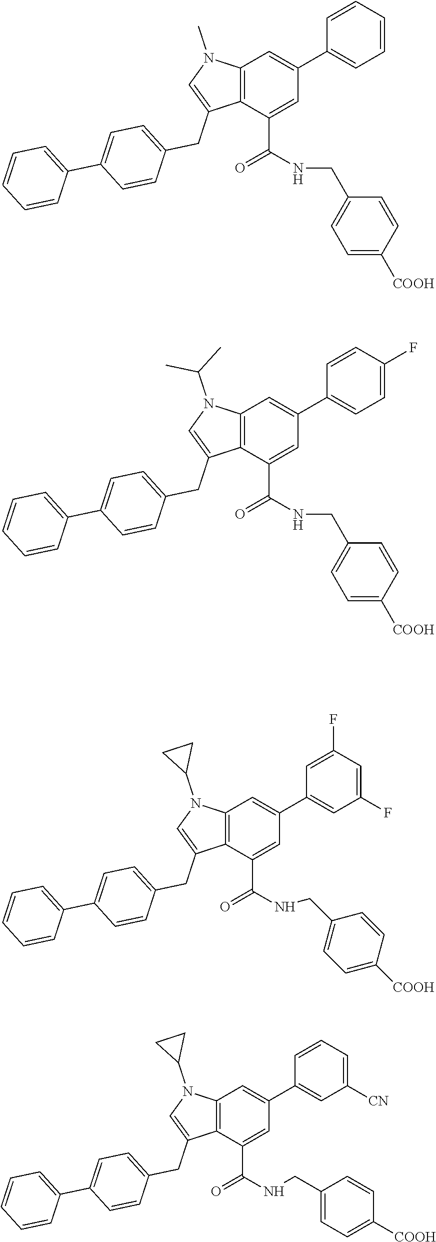 [SMILES: Cn1cc(Cc2ccc(-c3ccccc3)cc2)c2c(C(=O)NCc3ccc(C(=O)O)cc3)cc(-c3ccccc3)cc21 ; CC(C)n1cc(Cc2ccc(-c3ccccc3)cc2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Cc2ccc(-c4ccccc4)cc2)cn3C2CC2)cc1 ; N#Cc1cccc(-c2cc(C(=O)NCc3ccc(C(=O)O)cc3)c3c(Cc4ccc(-c5ccccc5)cc4)cn(C4CC4)c3c2)c1]

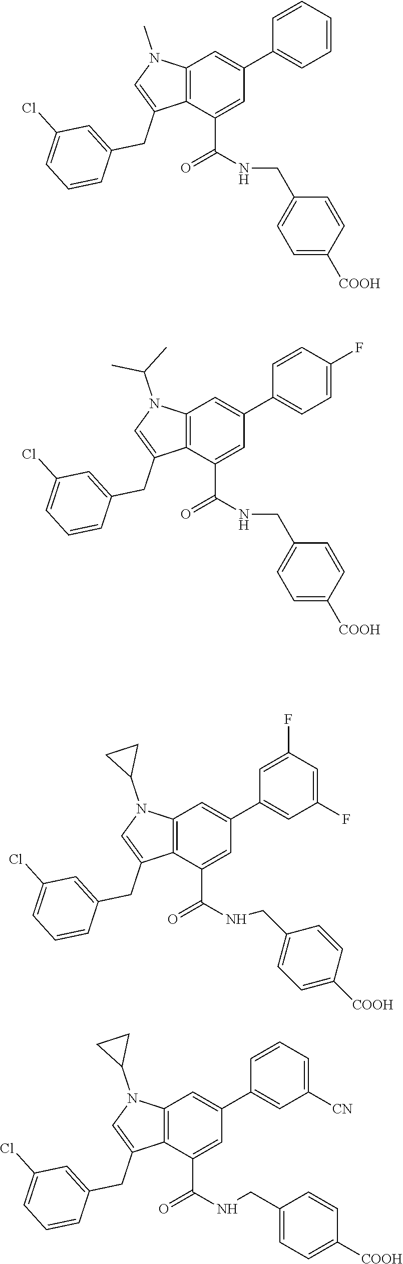 [SMILES: Cn1cc(Cc2cccc(Cl)c2)c2c(C(=O)NCc3ccc(C(=O)O)cc3)cc(-c3ccccc3)cc21 ; CC(C)n1cc(Cc2cccc(Cl)c2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Cc2cccc(Cl)c2)cn3C2CC2)cc1 ; N#Cc1cccc(-c2cc(C(=O)NCc3ccc(C(=O)O)cc3)c3c(Cc4cccc(Cl)c4)cn(C4CC4)c3c2)c1]

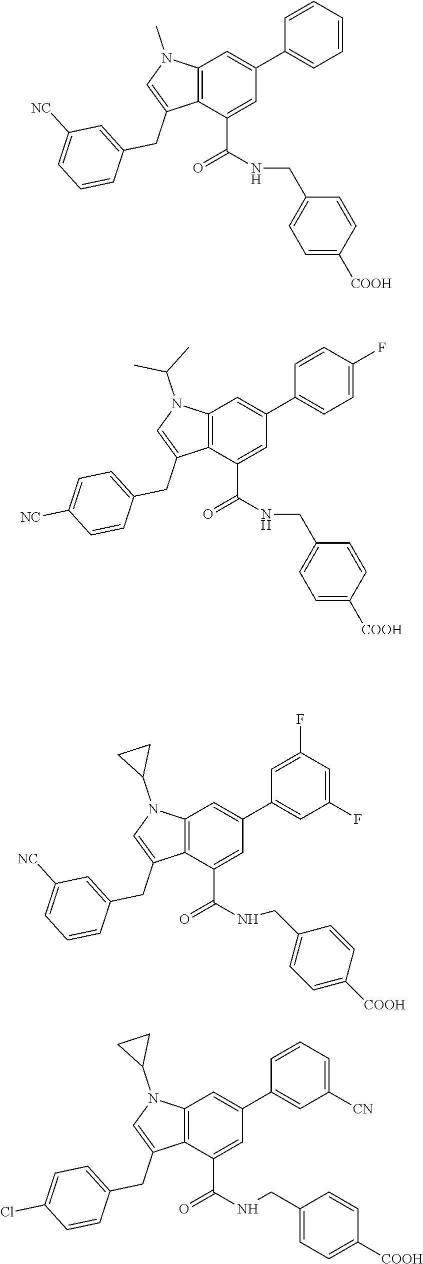 [SMILES: Cn1cc(Cc2cccc(C#N)c2)c2c(C(=O)NCc3ccc(C(=O)O)cc3)cc(-c3ccccc3)cc21 ; CC(C)n1cc(Cc2ccc(C#N)cc2)c2c(C(=O)NCc3ccc(C(=O)O)cc3)cc(-c3ccc(F)cc3)cc21 ; N#Cc1cccc(Cc2cn(C3CC3)c3cc(-c4cc(F)cc(F)c4)cc(C(=O)NCc4ccc(C(=O)O)cc4)c23)c1 ; N#Cc1cccc(-c2cc(C(=O)NCc3ccc(C(=O)O)cc3)c3c(Cc4ccc(Cl)cc4)cn(C4CC4)c3c2)c1]

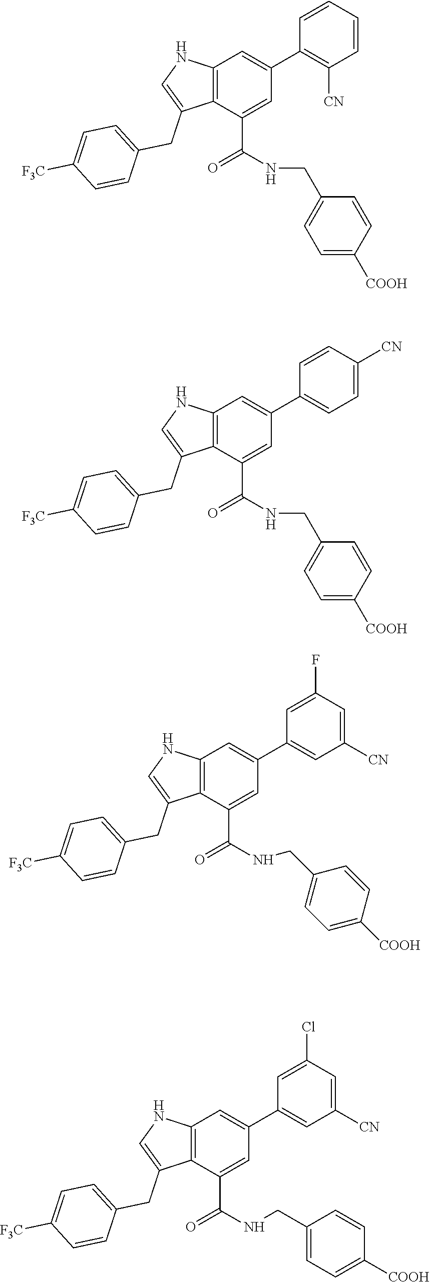 [SMILES: N#Cc1ccccc1-c1cc(C(=O)NCc2ccc(C(=O)O)cc2)c2c(Cc3ccc(C(F)(F)F)cc3)c[nH]c2c1 ; N#Cc1ccc(-c2cc(C(=O)NCc3ccc(C(=O)O)cc3)c3c(Cc4ccc(C(F)(F)F)cc4)c[nH]c3c2)cc1 ; N#Cc1cc(F)cc(-c2cc(C(=O)NCc3ccc(C(=O)O)cc3)c3c(Cc4ccc(C(F)(F)F)cc4)c[nH]c3c2)c1 ; N#Cc1cc(Cl)cc(-c2cc(C(=O)NCc3ccc(C(=O)O)cc3)c3c(Cc4ccc(C(F)(F)F)cc4)c[nH]c3c2)c1]

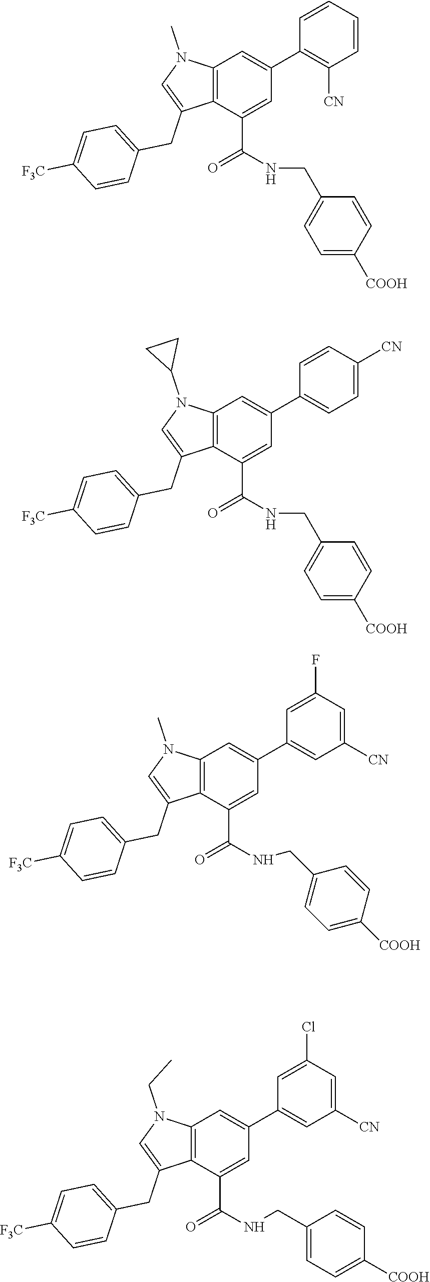 [SMILES: Cn1cc(Cc2ccc(C(F)(F)F)cc2)c2c(C(=O)NCc3ccc(C(=O)O)cc3)cc(-c3ccccc3C#N)cc21 ; N#Cc1ccc(-c2cc(C(=O)NCc3ccc(C(=O)O)cc3)c3c(Cc4ccc(C(F)(F)F)cc4)cn(C4CC4)c3c2)cc1 ; Cn1cc(Cc2ccc(C(F)(F)F)cc2)c2c(C(=O)NCc3ccc(C(=O)O)cc3)cc(-c3cc(F)cc(C#N)c3)cc21 ; CCn1cc(Cc2ccc(C(F)(F)F)cc2)c2c(C(=O)NCc3ccc(C(=O)O)cc3)cc(-c3cc(Cl)cc(C#N)c3)cc21]

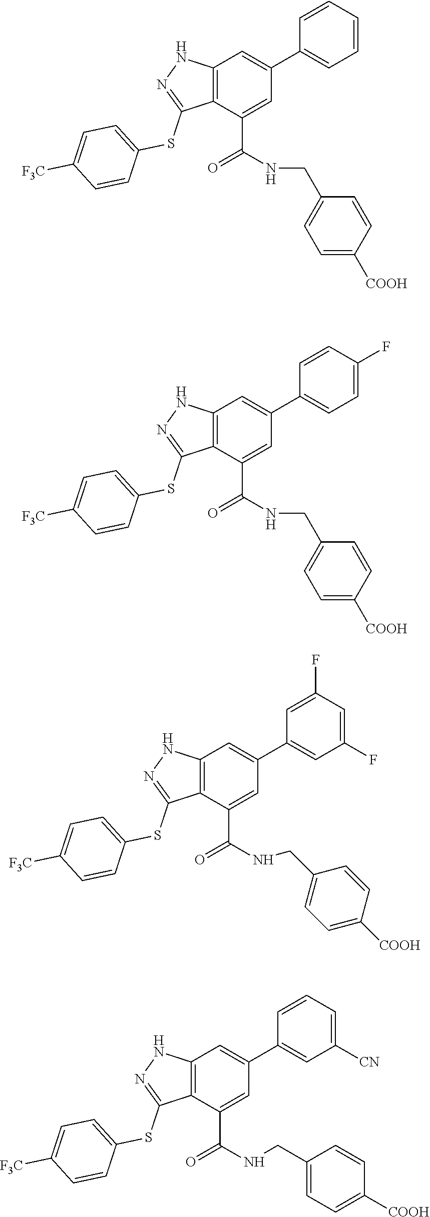 [SMILES: O=C(O)c1ccc(CNC(=O)c2cc(-c3ccccc3)cc3[nH]nc(Sc4ccc(C(F)(F)F)cc4)c23)cc1 ; O=C(O)c1ccc(CNC(=O)c2cc(-c3ccc(F)cc3)cc3[nH]nc(Sc4ccc(C(F)(F)F)cc4)c23)cc1 ; O=C(O)c1ccc(CNC(=O)c2cc(-c3cc(F)cc(F)c3)cc3[nH]nc(Sc4ccc(C(F)(F)F)cc4)c23)cc1 ; N#Cc1cccc(-c2cc(C(=O)NCc3ccc(C(=O)O)cc3)c3c(Sc4ccc(C(F)(F)F)cc4)n[nH]c3c2)c1]

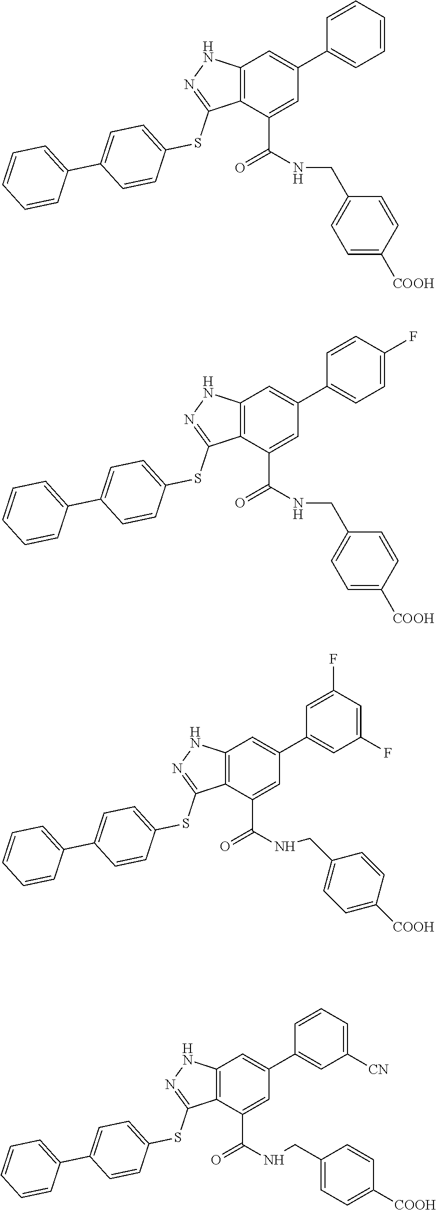 [SMILES: O=C(O)c1ccc(CNC(=O)c2cc(-c3ccccc3)cc3[nH]nc(Sc4ccc(-c5ccccc5)cc4)c23)cc1 ; O=C(O)c1ccc(CNC(=O)c2cc(-c3ccc(F)cc3)cc3[nH]nc(Sc4ccc(-c5ccccc5)cc4)c23)cc1 ; O=C(O)c1ccc(CNC(=O)c2cc(-c3cc(F)cc(F)c3)cc3[nH]nc(Sc4ccc(-c5ccccc5)cc4)c23)cc1 ; N#Cc1cccc(-c2cc(C(=O)NCc3ccc(C(=O)O)cc3)c3c(Sc4ccc(-c5ccccc5)cc4)n[nH]c3c2)c1]

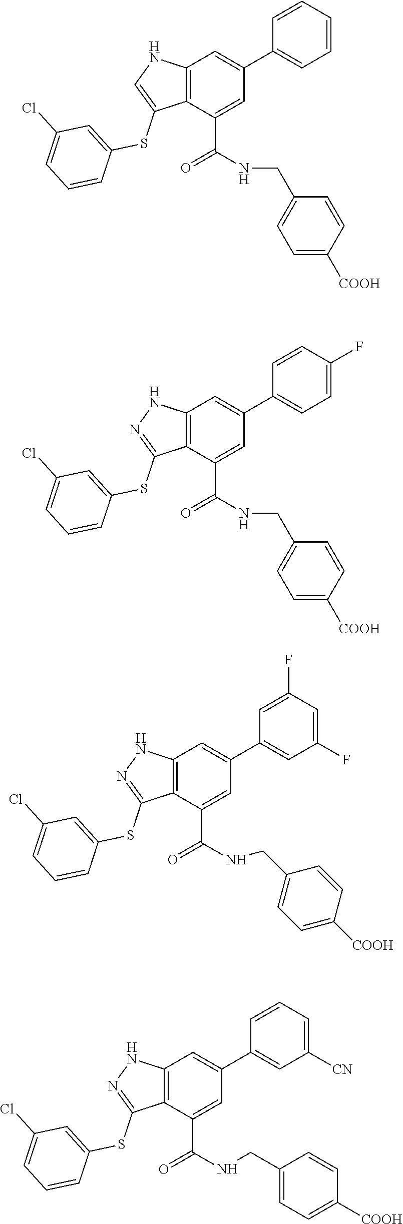 [SMILES: O=C(O)c1ccc(CNC(=O)c2cc(-c3ccccc3)cc3[nH]cc(Sc4cccc(Cl)c4)c23)cc1 ; O=C(O)c1ccc(CNC(=O)c2cc(-c3ccc(F)cc3)cc3[nH]nc(Sc4cccc(Cl)c4)c23)cc1 ; O=C(O)c1ccc(CNC(=O)c2cc(-c3cc(F)cc(F)c3)cc3[nH]nc(Sc4cccc(Cl)c4)c23)cc1 ; N#Cc1cccc(-c2cc(C(=O)NCc3ccc(C(=O)O)cc3)c3c(Sc4cccc(Cl)c4)n[nH]c3c2)c1]

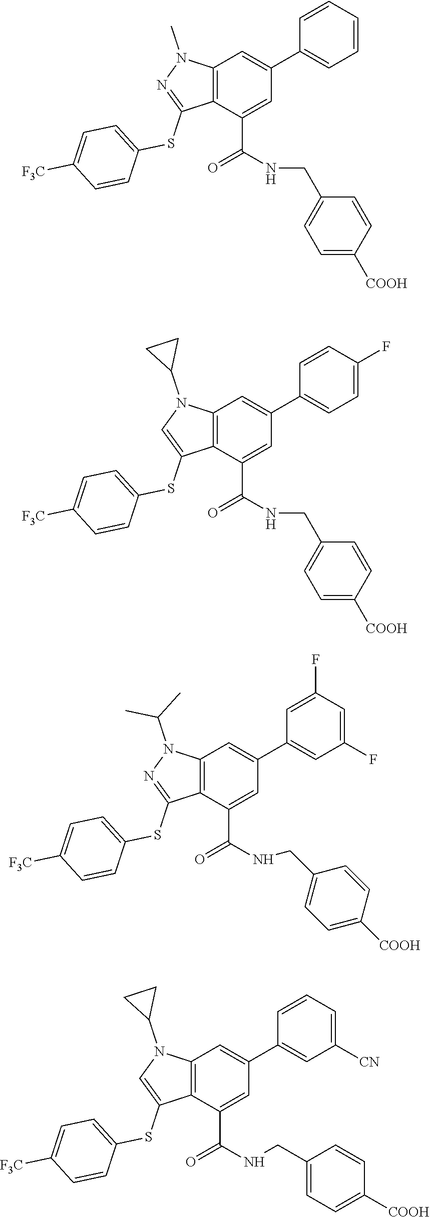 [SMILES: Cn1nc(Sc2ccc(C(F)(F)F)cc2)c2c(C(=O)NCc3ccc(C(=O)O)cc3)cc(-c3ccccc3)cc21 ; O=C(O)c1ccc(CNC(=O)c2cc(-c3ccc(F)cc3)cc3c2c(Sc2ccc(C(F)(F)F)cc2)cn3C2CC2)cc1 ; CC(C)n1nc(Sc2ccc(C(F)(F)F)cc2)c2c(C(=O)NCc3ccc(C(=O)O)cc3)cc(-c3cc(F)cc(F)c3)cc21 ; N#Cc1cccc(-c2cc(C(=O)NCc3ccc(C(=O)O)cc3)c3c(Sc4ccc(C(F)(F)F)cc4)cn(C4CC4)c3c2)c1]

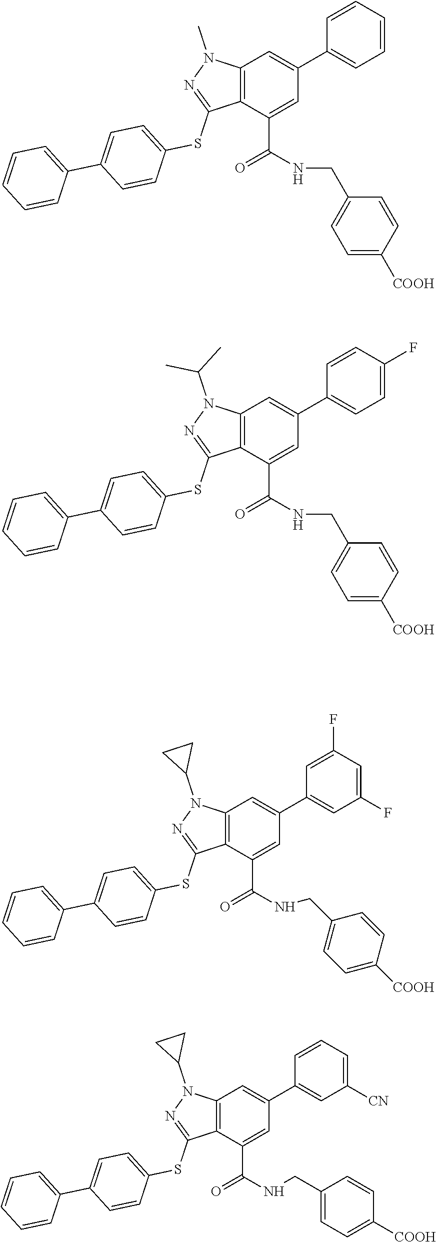 [SMILES: Cn1nc(Sc2ccc(-c3ccccc3)cc2)c2c(C(=O)NCc3ccc(C(=O)O)cc3)cc(-c3ccccc3)cc21 ; CC(C)n1nc(Sc2ccc(-c3ccccc3)cc2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Sc2ccc(-c4ccccc4)cc2)nn3C2CC2)cc1 ; N#Cc1cccc(-c2cc(C(=O)NCc3ccc(C(=O)O)cc3)c3c(Sc4ccc(-c5ccccc5)cc4)nn(C4CC4)c3c2)c1]

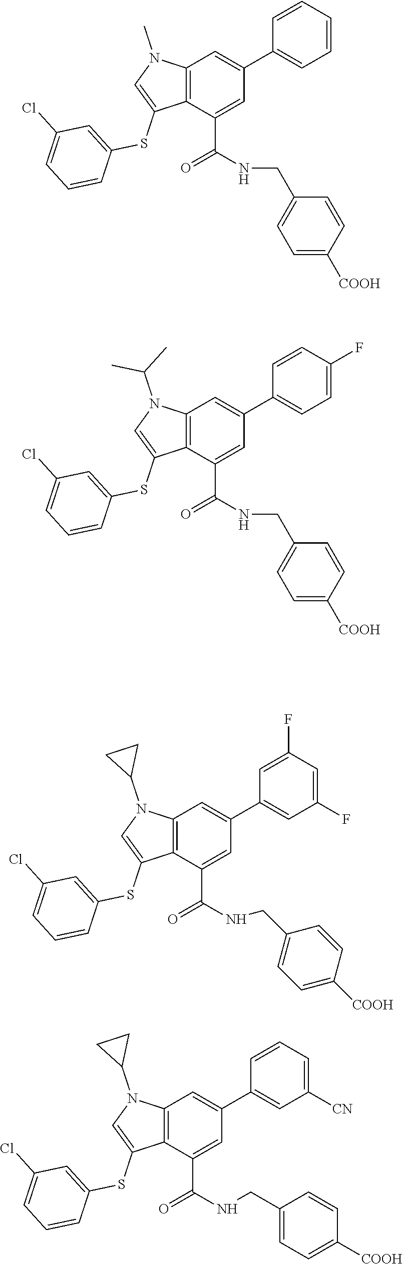 [SMILES: Cn1cc(Sc2cccc(Cl)c2)c2c(C(=O)NCc3ccc(C(=O)O)cc3)cc(-c3ccccc3)cc21 ; CC(C)n1cc(Sc2cccc(Cl)c2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Sc2cccc(Cl)c2)cn3C2CC2)cc1 ; N#Cc1cccc(-c2cc(C(=O)NCc3ccc(C(=O)O)cc3)c3c(Sc4cccc(Cl)c4)cn(C4CC4)c3c2)c1]

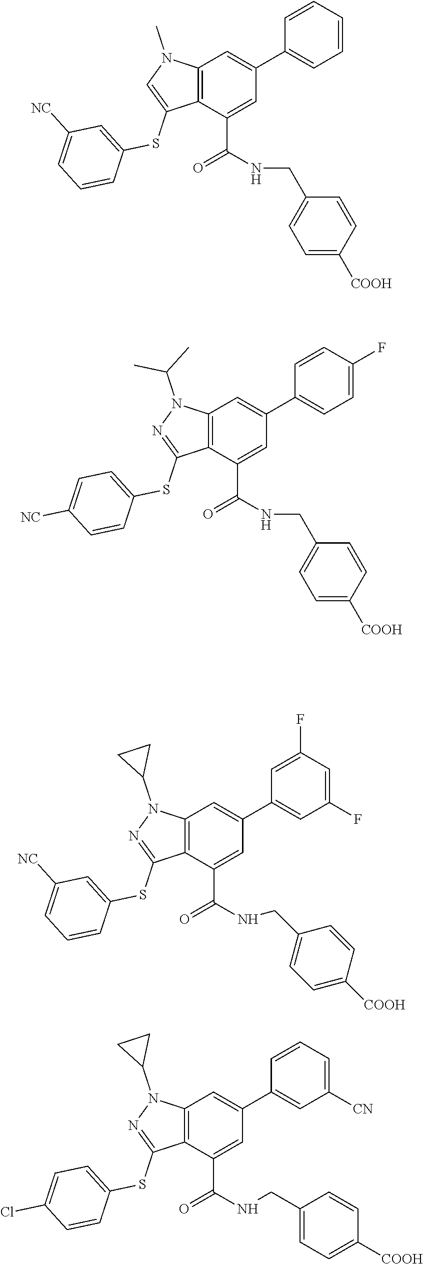 [SMILES: Cn1cc(Sc2cccc(C#N)c2)c2c(C(=O)NCc3ccc(C(=O)O)cc3)cc(-c3ccccc3)cc21 ; CC(C)n1nc(Sc2ccc(C#N)cc2)c2c(C(=O)NCc3ccc(C(=O)O)cc3)cc(-c3ccc(F)cc3)cc21 ; N#Cc1cccc(Sc2nn(C3CC3)c3cc(-c4cc(F)cc(F)c4)cc(C(=O)NCc4ccc(C(=O)O)cc4)c23)c1 ; N#Cc1cccc(-c2cc(C(=O)NCc3ccc(C(=O)O)cc3)c3c(Sc4ccc(Cl)cc4)nn(C4CC4)c3c2)c1]

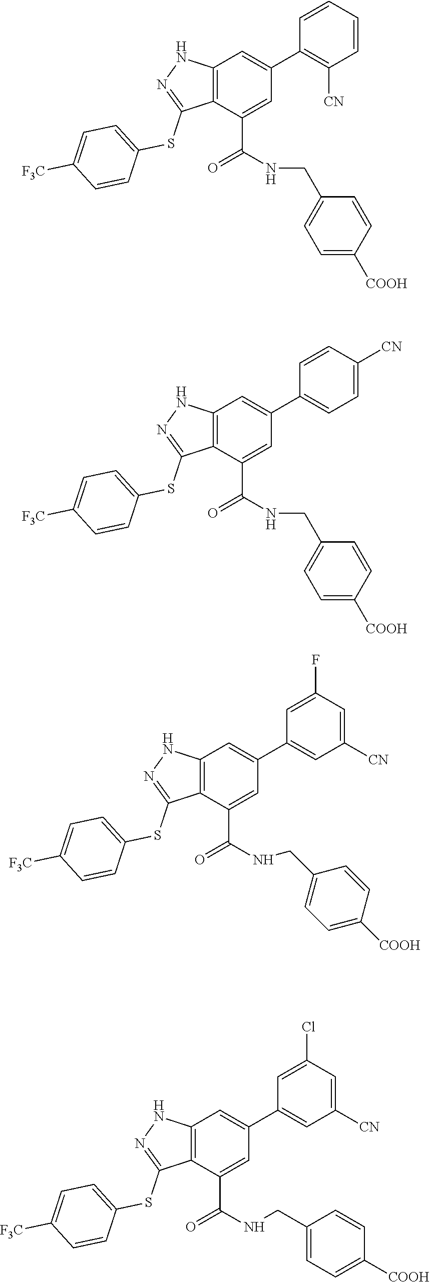 [SMILES: N#Cc1ccccc1-c1cc(C(=O)NCc2ccc(C(=O)O)cc2)c2c(Sc3ccc(C(F)(F)F)cc3)n[nH]c2c1 ; N#Cc1ccc(-c2cc(C(=O)NCc3ccc(C(=O)O)cc3)c3c(Sc4ccc(C(F)(F)F)cc4)n[nH]c3c2)cc1 ; N#Cc1cc(F)cc(-c2cc(C(=O)NCc3ccc(C(=O)O)cc3)c3c(Sc4ccc(C(F)(F)F)cc4)n[nH]c3c2)c1 ; N#Cc1cc(Cl)cc(-c2cc(C(=O)NCc3ccc(C(=O)O)cc3)c3c(Sc4ccc(C(F)(F)F)cc4)n[nH]c3c2)c1]

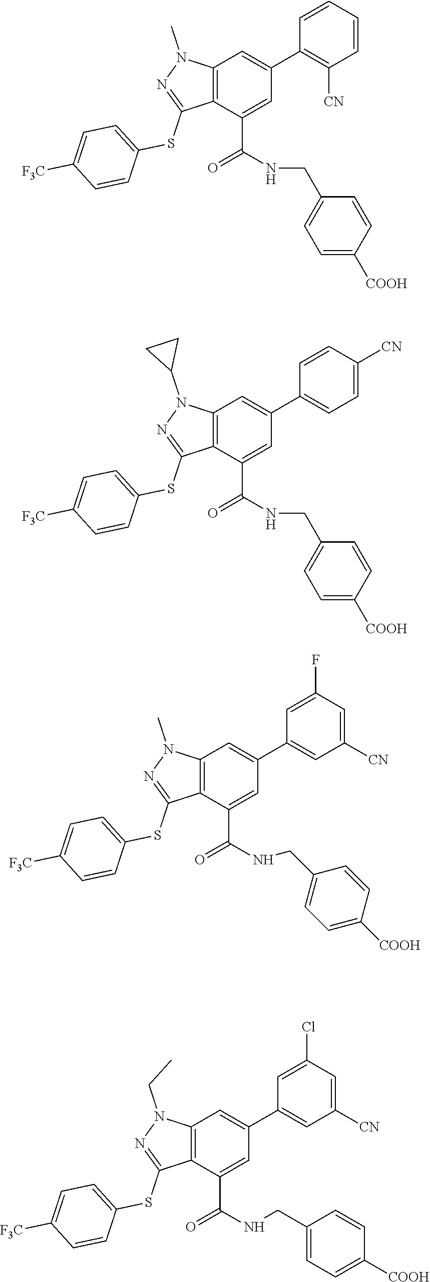 [SMILES: Cn1nc(Sc2ccc(C(F)(F)F)cc2)c2c(C(=O)NCc3ccc(C(=O)O)cc3)cc(-c3ccccc3C#N)cc21 ; N#Cc1ccc(-c2cc(C(=O)NCc3ccc(C(=O)O)cc3)c3c(Sc4ccc(C(F)(F)F)cc4)nn(C4CC4)c3c2)cc1 ; Cn1nc(Sc2ccc(C(F)(F)F)cc2)c2c(C(=O)NCc3ccc(C(=O)O)cc3)cc(-c3cc(F)cc(C#N)c3)cc21 ; CCn1nc(Sc2ccc(C(F)(F)F)cc2)c2c(C(=O)NCc3ccc(C(=O)O)cc3)cc(-c3cc(Cl)cc(C#N)c3)cc21]

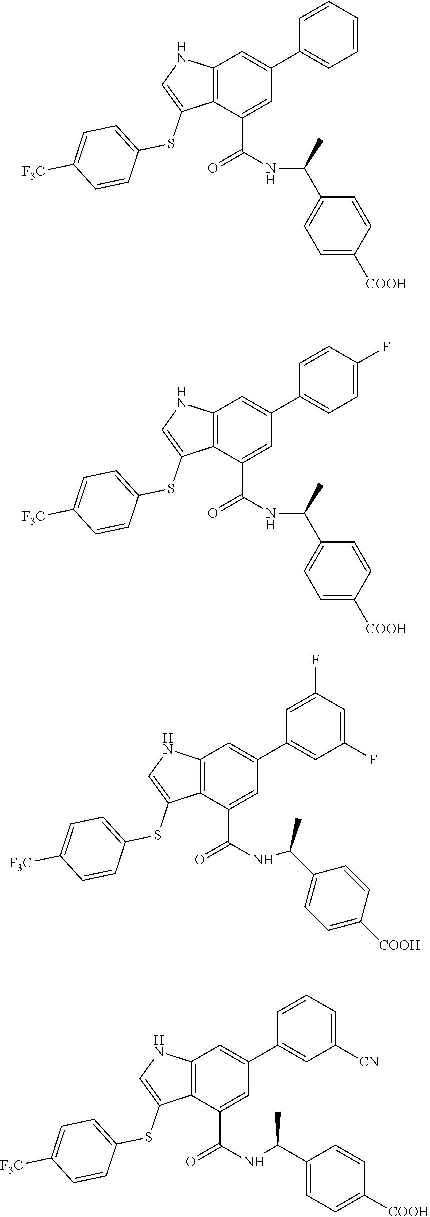 [SMILES: C[C@H](NC(=O)c1cc(-c2ccccc2)cc2[nH]cc(Sc3ccc(C(F)(F)F)cc3)c12)c1ccc(C(=O)O)cc1 ; C[C@H](NC(=O)c1cc(-c2ccc(F)cc2)cc2[nH]cc(Sc3ccc(C(F)(F)F)cc3)c12)c1ccc(C(=O)O)cc1 ; C[C@H](NC(=O)c1cc(-c2cc(F)cc(F)c2)cc2[nH]cc(Sc3ccc(C(F)(F)F)cc3)c12)c1ccc(C(=O)O)cc1 ; C[C@H](NC(=O)c1cc(-c2cccc(C#N)c2)cc2[nH]cc(Sc3ccc(C(F)(F)F)cc3)c12)c1ccc(C(=O)O)cc1]

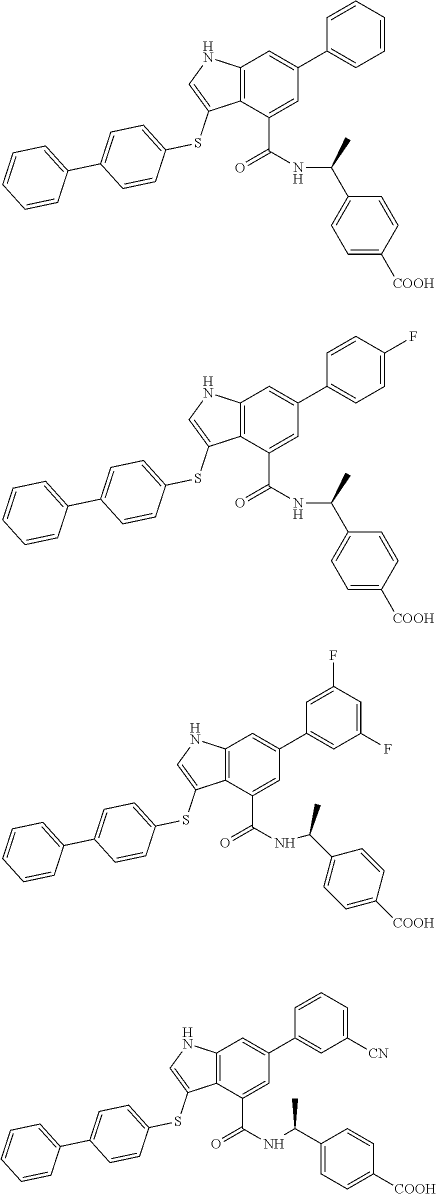 [SMILES: C[C@H](NC(=O)c1cc(-c2ccccc2)cc2[nH]cc(Sc3ccc(-c4ccccc4)cc3)c12)c1ccc(C(=O)O)cc1 ; C[C@H](NC(=O)c1cc(-c2ccc(F)cc2)cc2[nH]cc(Sc3ccc(-c4ccccc4)cc3)c12)c1ccc(C(=O)O)cc1 ; C[C@H](NC(=O)c1cc(-c2cc(F)cc(F)c2)cc2[nH]cc(Sc3ccc(-c4ccccc4)cc3)c12)c1ccc(C(=O)O)cc1 ; C[C@H](NC(=O)c1cc(-c2cccc(C#N)c2)cc2[nH]cc(Sc3ccc(-c4ccccc4)cc3)c12)c1ccc(C(=O)O)cc1]

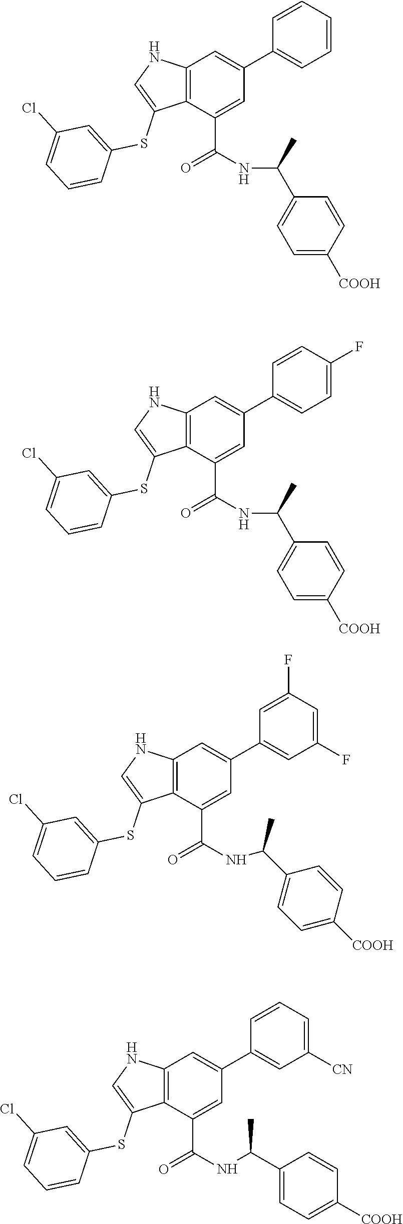 [SMILES: C[C@H](NC(=O)c1cc(-c2ccccc2)cc2[nH]cc(Sc3cccc(Cl)c3)c12)c1ccc(C(=O)O)cc1 ; C[C@H](NC(=O)c1cc(-c2ccc(F)cc2)cc2[nH]cc(Sc3cccc(Cl)c3)c12)c1ccc(C(=O)O)cc1 ; C[C@H](NC(=O)c1cc(-c2cc(F)cc(F)c2)cc2[nH]cc(Sc3cccc(Cl)c3)c12)c1ccc(C(=O)O)cc1 ; C[C@H](NC(=O)c1cc(-c2cccc(C#N)c2)cc2[nH]cc(Sc3cccc(Cl)c3)c12)c1ccc(C(=O)O)cc1]

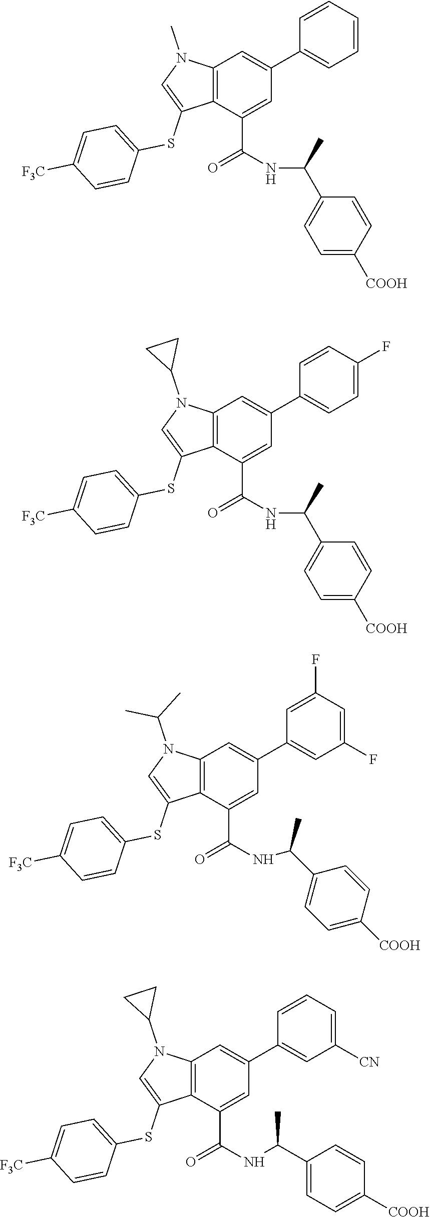 [SMILES: C[C@H](NC(=O)c1cc(-c2ccccc2)cc2c1c(Sc1ccc(C(F)(F)F)cc1)cn2C)c1ccc(C(=O)O)cc1 ; C[C@H](NC(=O)c1cc(-c2ccc(F)cc2)cc2c1c(Sc1ccc(C(F)(F)F)cc1)cn2C1CC1)c1ccc(C(=O)O)cc1 ; CC(C)n1cc(Sc2ccc(C(F)(F)F)cc2)c2c(C(=O)N[C@@H](C)c3ccc(C(=O)O)cc3)cc(-c3cc(F)cc(F)c3)cc21 ; C[C@H](NC(=O)c1cc(-c2cccc(C#N)c2)cc2c1c(Sc1ccc(C(F)(F)F)cc1)cn2C1CC1)c1ccc(C(=O)O)cc1]

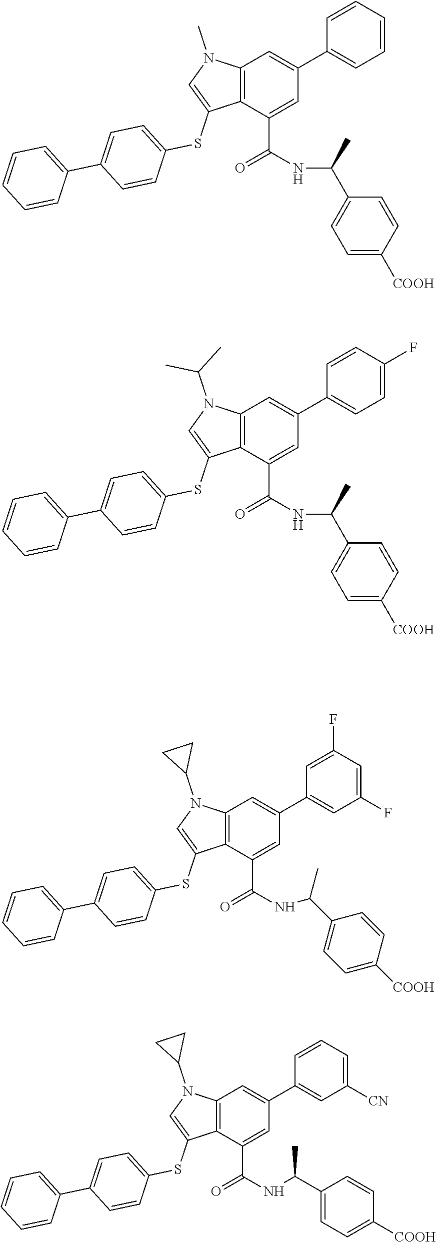 [SMILES: C[C@H](NC(=O)c1cc(-c2ccccc2)cc2c1c(Sc1ccc(-c3ccccc3)cc1)cn2C)c1ccc(C(=O)O)cc1 ; CC(C)n1cc(Sc2ccc(-c3ccccc3)cc2)c2c(C(=O)N[C@@H](C)c3ccc(C(=O)O)cc3)cc(-c3ccc(F)cc3)cc21 ; CC(NC(=O)c1cc(-c2cc(F)cc(F)c2)cc2c1c(Sc1ccc(-c3ccccc3)cc1)cn2C1CC1)c1ccc(C(=O)O)cc1 ; C[C@H](NC(=O)c1cc(-c2cccc(C#N)c2)cc2c1c(Sc1ccc(-c3ccccc3)cc1)cn2C1CC1)c1ccc(C(=O)O)cc1]

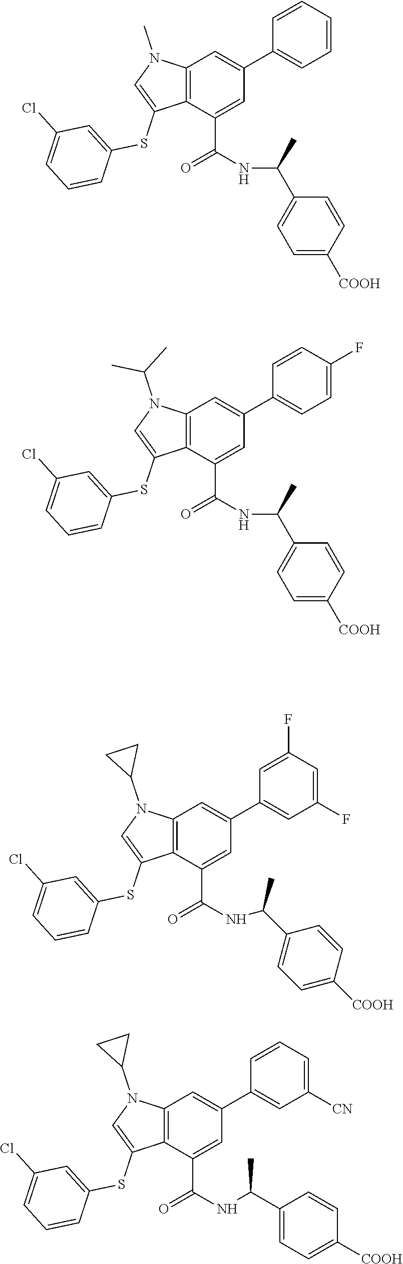 [SMILES: C[C@H](NC(=O)c1cc(-c2ccccc2)cc2c1c(Sc1cccc(Cl)c1)cn2C)c1ccc(C(=O)O)cc1 ; CC(C)n1cc(Sc2cccc(Cl)c2)c2c(C(=O)N[C@@H](C)c3ccc(C(=O)O)cc3)cc(-c3ccc(F)cc3)cc21 ; C[C@H](NC(=O)c1cc(-c2cc(F)cc(F)c2)cc2c1c(Sc1cccc(Cl)c1)cn2C1CC1)c1ccc(C(=O)O)cc1 ; C[C@H](NC(=O)c1cc(-c2cccc(C#N)c2)cc2c1c(Sc1cccc(Cl)c1)cn2C1CC1)c1ccc(C(=O)O)cc1]

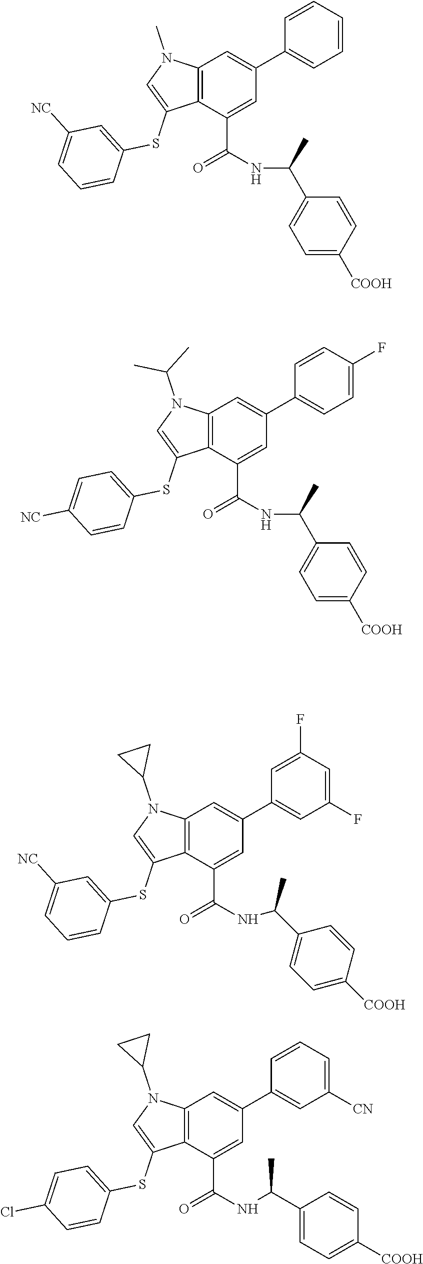 [SMILES: C[C@H](NC(=O)c1cc(-c2ccccc2)cc2c1c(Sc1cccc(C#N)c1)cn2C)c1ccc(C(=O)O)cc1 ; CC(C)n1cc(Sc2ccc(C#N)cc2)c2c(C(=O)N[C@@H](C)c3ccc(C(=O)O)cc3)cc(-c3ccc(F)cc3)cc21 ; C[C@H](NC(=O)c1cc(-c2cc(F)cc(F)c2)cc2c1c(Sc1cccc(C#N)c1)cn2C1CC1)c1ccc(C(=O)O)cc1 ; C[C@H](NC(=O)c1cc(-c2cccc(C#N)c2)cc2c1c(Sc1ccc(Cl)cc1)cn2C1CC1)c1ccc(C(=O)O)cc1]

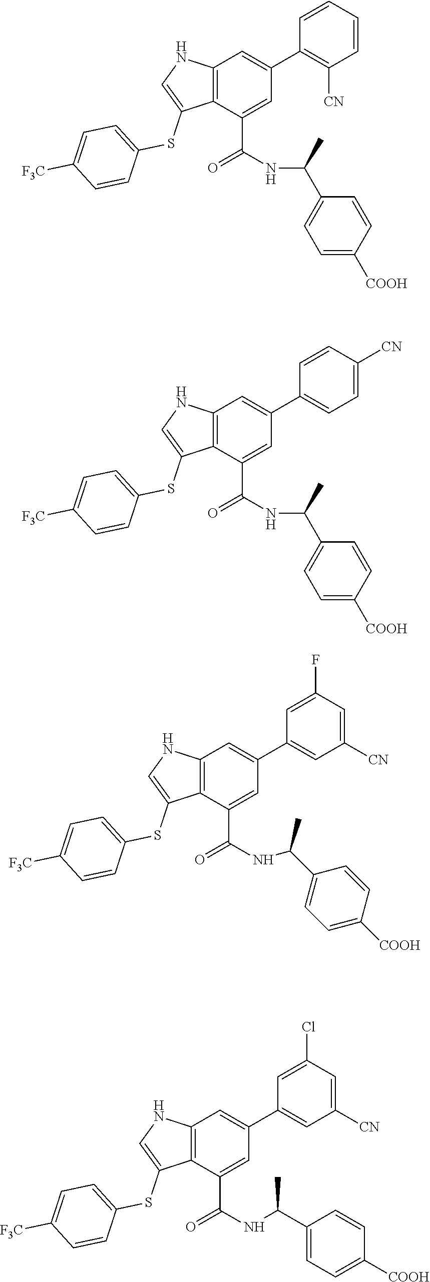 [SMILES: C[C@H](NC(=O)c1cc(-c2ccccc2C#N)cc2[nH]cc(Sc3ccc(C(F)(F)F)cc3)c12)c1ccc(C(=O)O)cc1 ; C[C@H](NC(=O)c1cc(-c2ccc(C#N)cc2)cc2[nH]cc(Sc3ccc(C(F)(F)F)cc3)c12)c1ccc(C(=O)O)cc1 ; C[C@H](NC(=O)c1cc(-c2cc(F)cc(C#N)c2)cc2[nH]cc(Sc3ccc(C(F)(F)F)cc3)c12)c1ccc(C(=O)O)cc1 ; C[C@H](NC(=O)c1cc(-c2cc(Cl)cc(C#N)c2)cc2[nH]cc(Sc3ccc(C(F)(F)F)cc3)c12)c1ccc(C(=O)O)cc1]

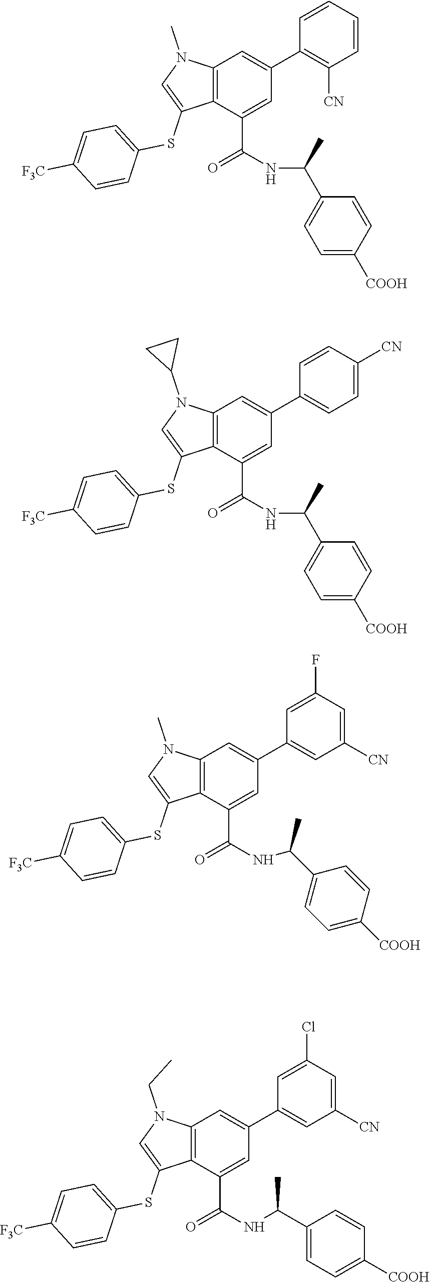 [SMILES: C[C@H](NC(=O)c1cc(-c2ccccc2C#N)cc2c1c(Sc1ccc(C(F)(F)F)cc1)cn2C)c1ccc(C(=O)O)cc1 ; C[C@H](NC(=O)c1cc(-c2ccc(C#N)cc2)cc2c1c(Sc1ccc(C(F)(F)F)cc1)cn2C1CC1)c1ccc(C(=O)O)cc1 ; C[C@H](NC(=O)c1cc(-c2cc(F)cc(C#N)c2)cc2c1c(Sc1ccc(C(F)(F)F)cc1)cn2C)c1ccc(C(=O)O)cc1 ; CCn1cc(Sc2ccc(C(F)(F)F)cc2)c2c(C(=O)N[C@@H](C)c3ccc(C(=O)O)cc3)cc(-c3cc(Cl)cc(C#N)c3)cc21]

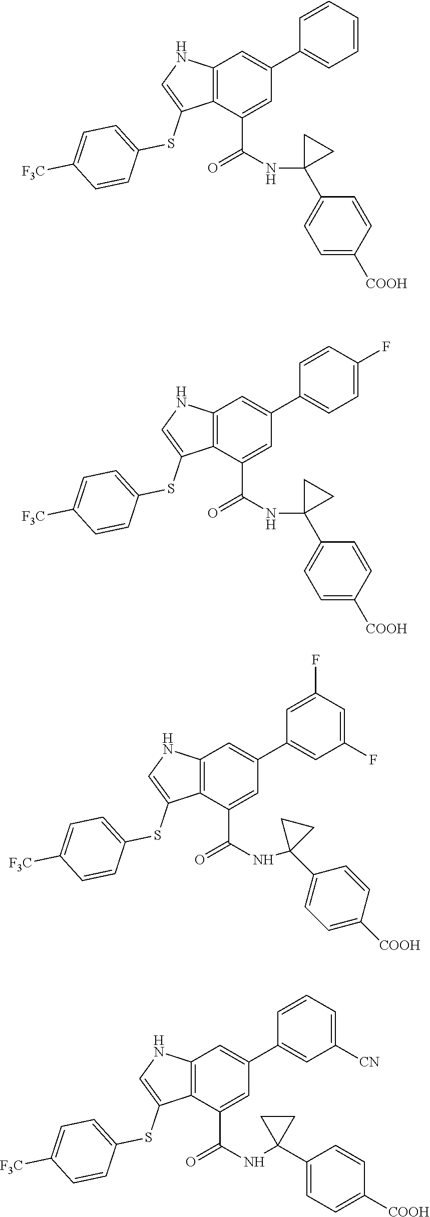 [SMILES: O=C(O)c1ccc(C2(NC(=O)c3cc(-c4ccccc4)cc4[nH]cc(Sc5ccc(C(F)(F)F)cc5)c34)CC2)cc1 ; O=C(O)c1ccc(C2(NC(=O)c3cc(-c4ccc(F)cc4)cc4[nH]cc(Sc5ccc(C(F)(F)F)cc5)c34)CC2)cc1 ; O=C(O)c1ccc(C2(NC(=O)c3cc(-c4cc(F)cc(F)c4)cc4[nH]cc(Sc5ccc(C(F)(F)F)cc5)c34)CC2)cc1 ; N#Cc1cccc(-c2cc(C(=O)NC3(c4ccc(C(=O)O)cc4)CC3)c3c(Sc4ccc(C(F)(F)F)cc4)c[nH]c3c2)c1]

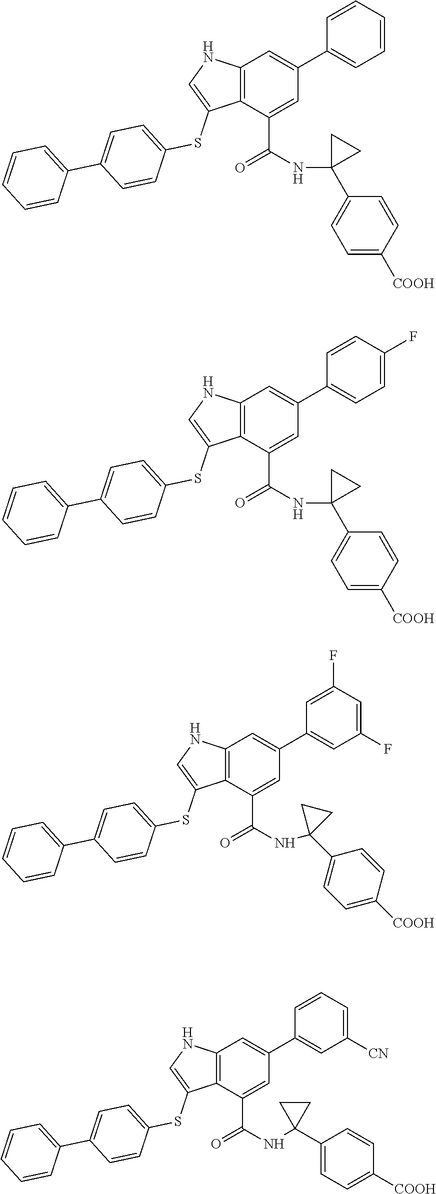 [SMILES: O=C(O)c1ccc(C2(NC(=O)c3cc(-c4ccccc4)cc4[nH]cc(Sc5ccc(-c6ccccc6)cc5)c34)CC2)cc1 ; O=C(O)c1ccc(C2(NC(=O)c3cc(-c4ccc(F)cc4)cc4[nH]cc(Sc5ccc(-c6ccccc6)cc5)c34)CC2)cc1 ; O=C(O)c1ccc(C2(NC(=O)c3cc(-c4cc(F)cc(F)c4)cc4[nH]cc(Sc5ccc(-c6ccccc6)cc5)c34)CC2)cc1 ; N#Cc1cccc(-c2cc(C(=O)NC3(c4ccc(C(=O)O)cc4)CC3)c3c(Sc4ccc(-c5ccccc5)cc4)c[nH]c3c2)c1]

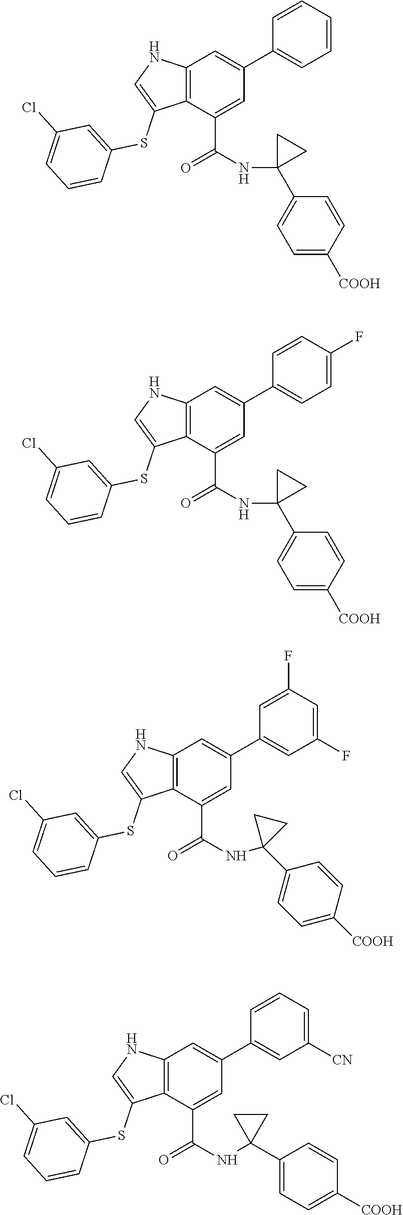 [SMILES: O=C(O)c1ccc(C2(NC(=O)c3cc(-c4ccccc4)cc4[nH]cc(Sc5cccc(Cl)c5)c34)CC2)cc1 ; O=C(O)c1ccc(C2(NC(=O)c3cc(-c4ccc(F)cc4)cc4[nH]cc(Sc5cccc(Cl)c5)c34)CC2)cc1 ; O=C(O)c1ccc(C2(NC(=O)c3cc(-c4cc(F)cc(F)c4)cc4[nH]cc(Sc5cccc(Cl)c5)c34)CC2)cc1 ; N#Cc1cccc(-c2cc(C(=O)NC3(c4ccc(C(=O)O)cc4)CC3)c3c(Sc4cccc(Cl)c4)c[nH]c3c2)c1]

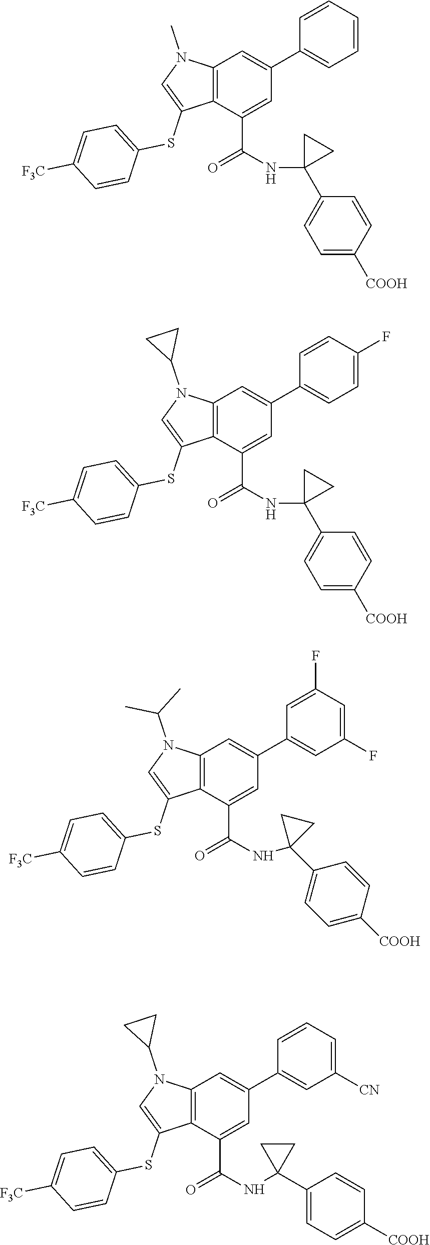 [SMILES: Cn1cc(Sc2ccc(C(F)(F)F)cc2)c2c(C(=O)NC3(c4ccc(C(=O)O)cc4)CC3)cc(-c3ccccc3)cc21 ; O=C(O)c1ccc(C2(NC(=O)c3cc(-c4ccc(F)cc4)cc4c3c(Sc3ccc(C(F)(F)F)cc3)cn4C3CC3)CC2)cc1 ; CC(C)n1cc(Sc2ccc(C(F)(F)F)cc2)c2c(C(=O)NC3(c4ccc(C(=O)O)cc4)CC3)cc(-c3cc(F)cc(F)c3)cc21 ; N#Cc1cccc(-c2cc(C(=O)NC3(c4ccc(C(=O)O)cc4)CC3)c3c(Sc4ccc(C(F)(F)F)cc4)cn(C4CC4)c3c2)c1]

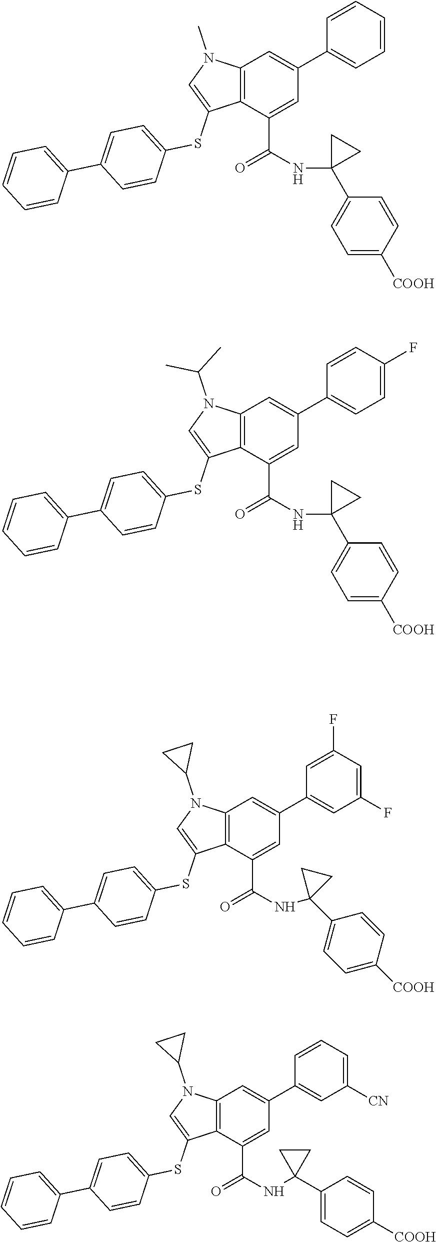 [SMILES: Cn1cc(Sc2ccc(-c3ccccc3)cc2)c2c(C(=O)NC3(c4ccc(C(=O)O)cc4)CC3)cc(-c3ccccc3)cc21 ; CC(C)n1cc(Sc2ccc(-c3ccccc3)cc2)c2c(C(=O)NC3(c4ccc(C(=O)O)cc4)CC3)cc(-c3ccc(F)cc3)cc21 ; O=C(O)c1ccc(C2(NC(=O)c3cc(-c4cc(F)cc(F)c4)cc4c3c(Sc3ccc(-c5ccccc5)cc3)cn4C3CC3)CC2)cc1 ; N#Cc1cccc(-c2cc(C(=O)NC3(c4ccc(C(=O)O)cc4)CC3)c3c(Sc4ccc(-c5ccccc5)cc4)cn(C4CC4)c3c2)c1]

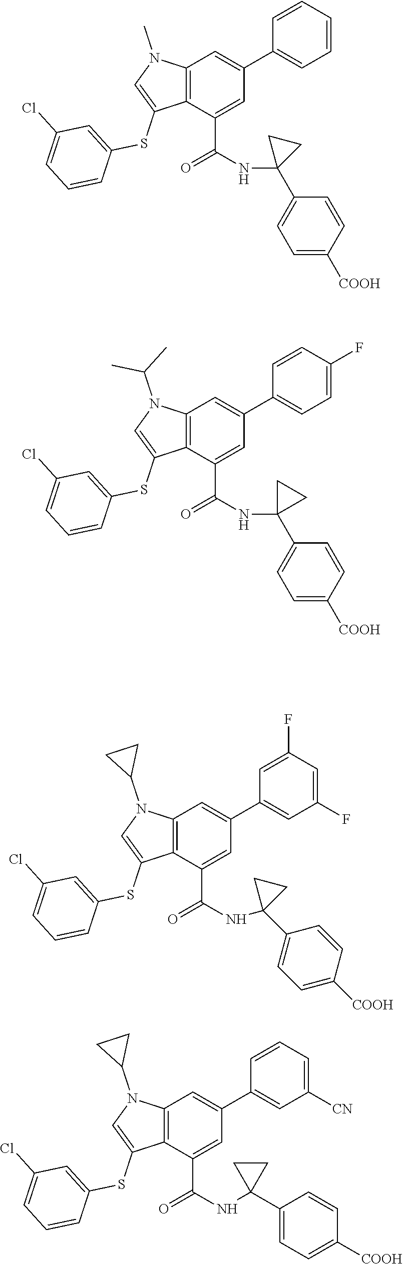 [SMILES: Cn1cc(Sc2cccc(Cl)c2)c2c(C(=O)NC3(c4ccc(C(=O)O)cc4)CC3)cc(-c3ccccc3)cc21 ; CC(C)n1cc(Sc2cccc(Cl)c2)c2c(C(=O)NC3(c4ccc(C(=O)O)cc4)CC3)cc(-c3ccc(F)cc3)cc21 ; O=C(O)c1ccc(C2(NC(=O)c3cc(-c4cc(F)cc(F)c4)cc4c3c(Sc3cccc(Cl)c3)cn4C3CC3)CC2)cc1 ; N#Cc1cccc(-c2cc(C(=O)NC3(c4ccc(C(=O)O)cc4)CC3)c3c(Sc4cccc(Cl)c4)cn(C4CC4)c3c2)c1]

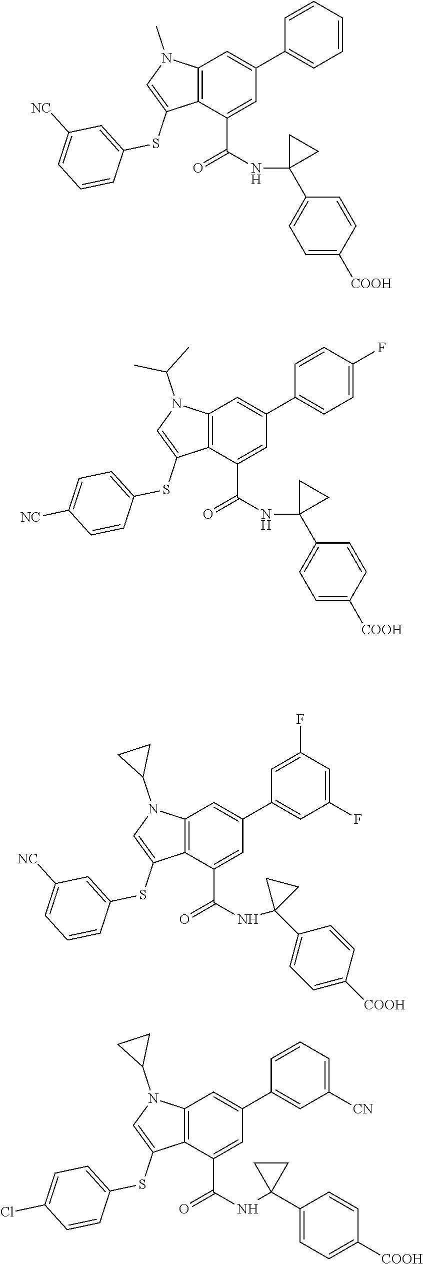 [SMILES: Cn1cc(Sc2cccc(C#N)c2)c2c(C(=O)NC3(c4ccc(C(=O)O)cc4)CC3)cc(-c3ccccc3)cc21 ; CC(C)n1cc(Sc2ccc(C#N)cc2)c2c(C(=O)NC3(c4ccc(C(=O)O)cc4)CC3)cc(-c3ccc(F)cc3)cc21 ; N#Cc1cccc(Sc2cn(C3CC3)c3cc(-c4cc(F)cc(F)c4)cc(C(=O)NC4(c5ccc(C(=O)O)cc5)CC4)c23)c1 ; N#Cc1cccc(-c2cc(C(=O)NC3(c4ccc(C(=O)O)cc4)CC3)c3c(Sc4ccc(Cl)cc4)cn(C4CC4)c3c2)c1]

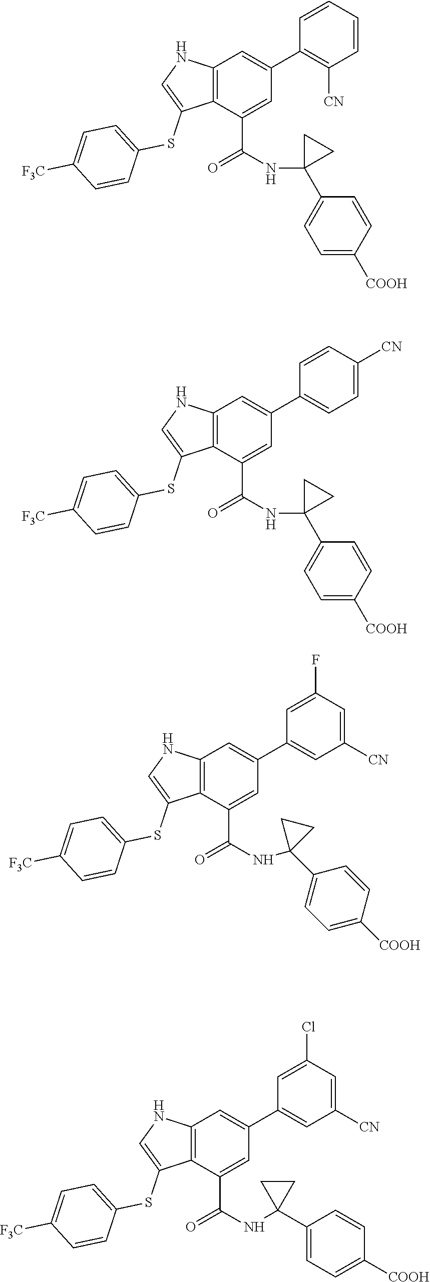 [SMILES: N#Cc1ccccc1-c1cc(C(=O)NC2(c3ccc(C(=O)O)cc3)CC2)c2c(Sc3ccc(C(F)(F)F)cc3)c[nH]c2c1 ; N#Cc1ccc(-c2cc(C(=O)NC3(c4ccc(C(=O)O)cc4)CC3)c3c(Sc4ccc(C(F)(F)F)cc4)c[nH]c3c2)cc1 ; N#Cc1cc(F)cc(-c2cc(C(=O)NC3(c4ccc(C(=O)O)cc4)CC3)c3c(Sc4ccc(C(F)(F)F)cc4)c[nH]c3c2)c1 ; N#Cc1cc(Cl)cc(-c2cc(C(=O)NC3(c4ccc(C(=O)O)cc4)CC3)c3c(Sc4ccc(C(F)(F)F)cc4)c[nH]c3c2)c1]

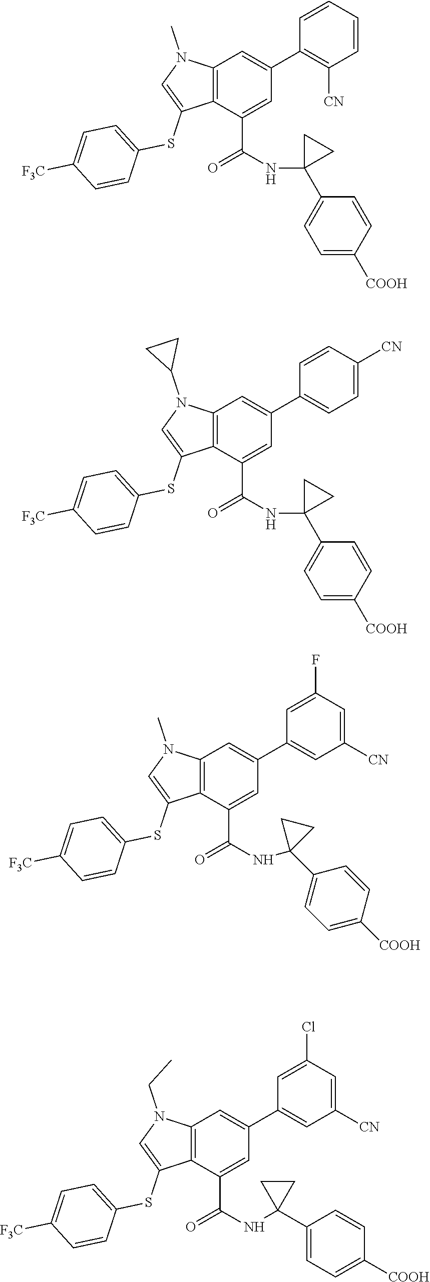 [SMILES: Cn1cc(Sc2ccc(C(F)(F)F)cc2)c2c(C(=O)NC3(c4ccc(C(=O)O)cc4)CC3)cc(-c3ccccc3C#N)cc21 ; N#Cc1ccc(-c2cc(C(=O)NC3(c4ccc(C(=O)O)cc4)CC3)c3c(Sc4ccc(C(F)(F)F)cc4)cn(C4CC4)c3c2)cc1 ; Cn1cc(Sc2ccc(C(F)(F)F)cc2)c2c(C(=O)NC3(c4ccc(C(=O)O)cc4)CC3)cc(-c3cc(F)cc(C#N)c3)cc21 ; CCn1cc(Sc2ccc(C(F)(F)F)cc2)c2c(C(=O)NC3(c4ccc(C(=O)O)cc4)CC3)cc(-c3cc(Cl)cc(C#N)c3)cc21]

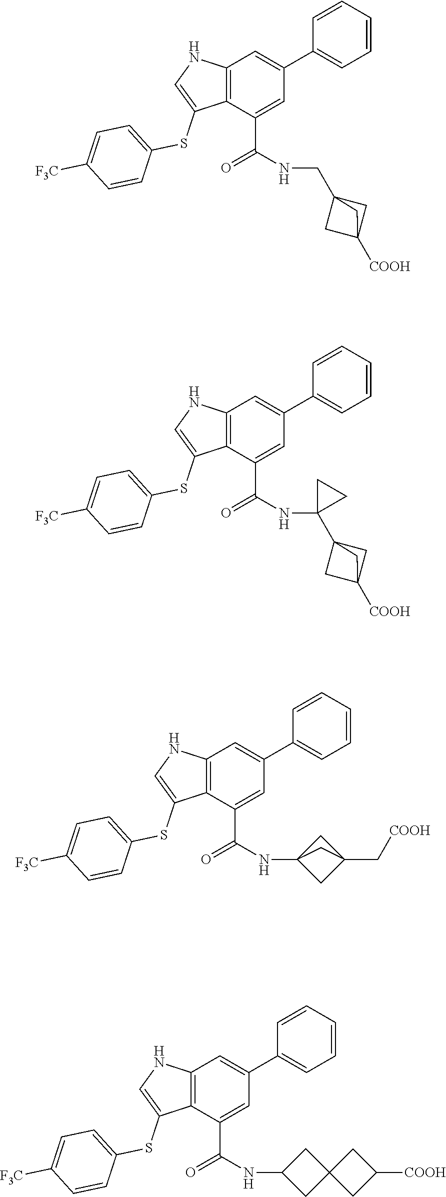 [SMILES: O=C(NCC12CC(C(=O)O)(C1)C2)c1cc(-c2ccccc2)cc2[nH]cc(Sc3ccc(C(F)(F)F)cc3)c12 ; O=C(NC1(C23CC(C(=O)O)(C2)C3)CC1)c1cc(-c2ccccc2)cc2[nH]cc(Sc3ccc(C(F)(F)F)cc3)c12 ; O=C(O)CC12CC(NC(=O)c3cc(-c4ccccc4)cc4[nH]cc(Sc5ccc(C(F)(F)F)cc5)c34)(C1)C2 ; O=C(NC1CC2(C1)CC(C(=O)O)C2)c1cc(-c2ccccc2)cc2[nH]cc(Sc3ccc(C(F)(F)F)cc3)c12]

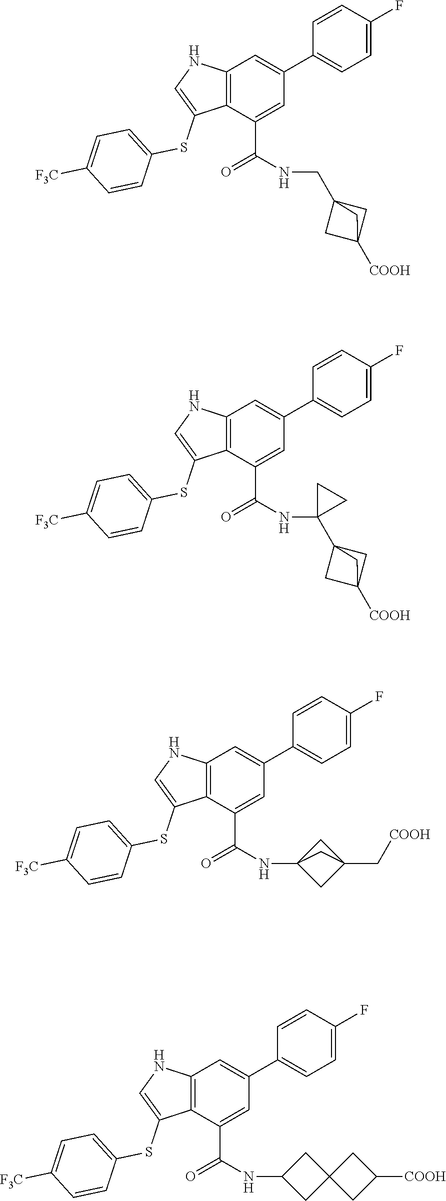 [SMILES: O=C(NCC12CC(C(=O)O)(C1)C2)c1cc(-c2ccc(F)cc2)cc2[nH]cc(Sc3ccc(C(F)(F)F)cc3)c12 ; O=C(NC1(C23CC(C(=O)O)(C2)C3)CC1)c1cc(-c2ccc(F)cc2)cc2[nH]cc(Sc3ccc(C(F)(F)F)cc3)c12 ; O=C(O)CC12CC(NC(=O)c3cc(-c4ccc(F)cc4)cc4[nH]cc(Sc5ccc(C(F)(F)F)cc5)c34)(C1)C2 ; O=C(NC1CC2(C1)CC(C(=O)O)C2)c1cc(-c2ccc(F)cc2)cc2[nH]cc(Sc3ccc(C(F)(F)F)cc3)c12]

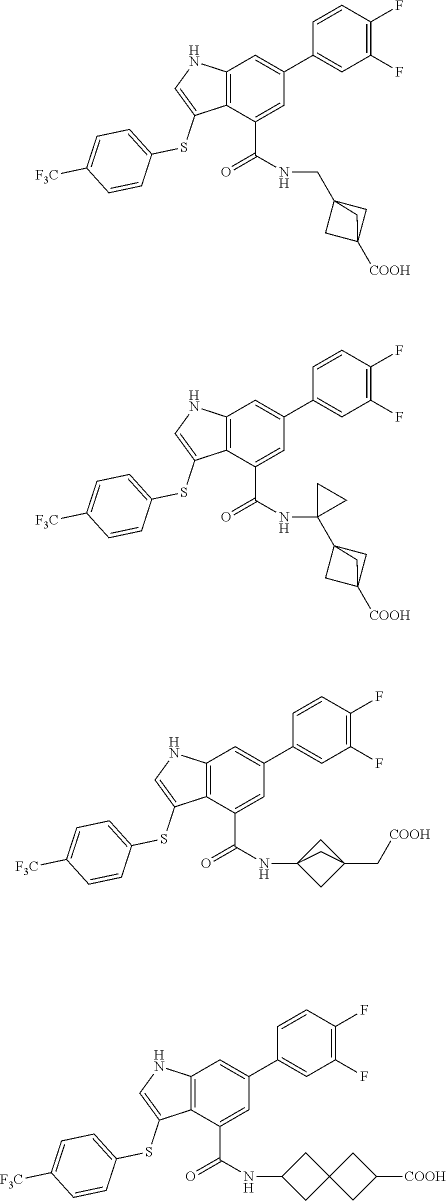 [SMILES: O=C(NCC12CC(C(=O)O)(C1)C2)c1cc(-c2ccc(F)c(F)c2)cc2[nH]cc(Sc3ccc(C(F)(F)F)cc3)c12 ; O=C(NC1(C23CC(C(=O)O)(C2)C3)CC1)c1cc(-c2ccc(F)c(F)c2)cc2[nH]cc(Sc3ccc(C(F)(F)F)cc3)c12 ; O=C(O)CC12CC(NC(=O)c3cc(-c4ccc(F)c(F)c4)cc4[nH]cc(Sc5ccc(C(F)(F)F)cc5)c34)(C1)C2 ; O=C(NC1CC2(C1)CC(C(=O)O)C2)c1cc(-c2ccc(F)c(F)c2)cc2[nH]cc(Sc3ccc(C(F)(F)F)cc3)c12]

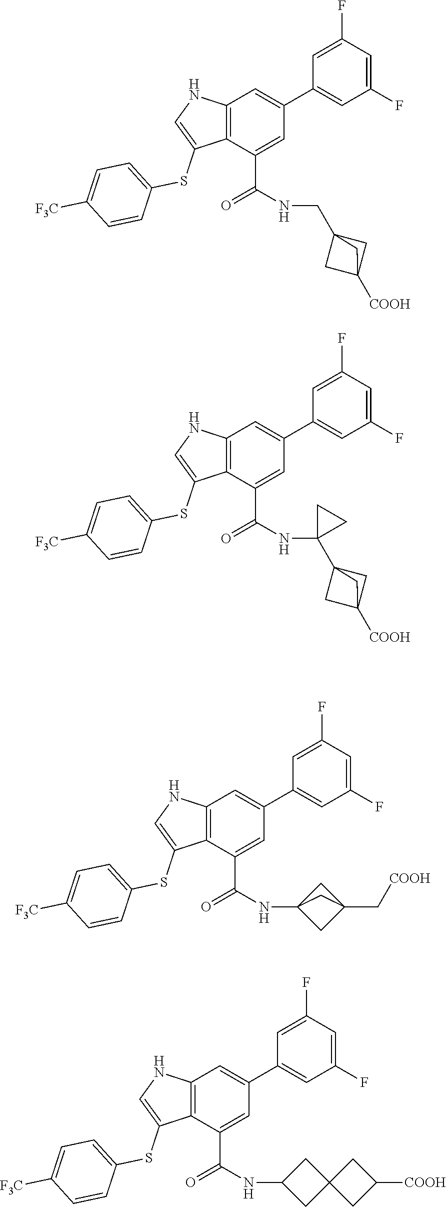 [SMILES: O=C(NCC12CC(C(=O)O)(C1)C2)c1cc(-c2cc(F)cc(F)c2)cc2[nH]cc(Sc3ccc(C(F)(F)F)cc3)c12 ; O=C(NC1(C23CC(C(=O)O)(C2)C3)CC1)c1cc(-c2cc(F)cc(F)c2)cc2[nH]cc(Sc3ccc(C(F)(F)F)cc3)c12 ; O=C(O)CC12CC(NC(=O)c3cc(-c4cc(F)cc(F)c4)cc4[nH]cc(Sc5ccc(C(F)(F)F)cc5)c34)(C1)C2 ; O=C(NC1CC2(C1)CC(C(=O)O)C2)c1cc(-c2cc(F)cc(F)c2)cc2[nH]cc(Sc3ccc(C(F)(F)F)cc3)c12]

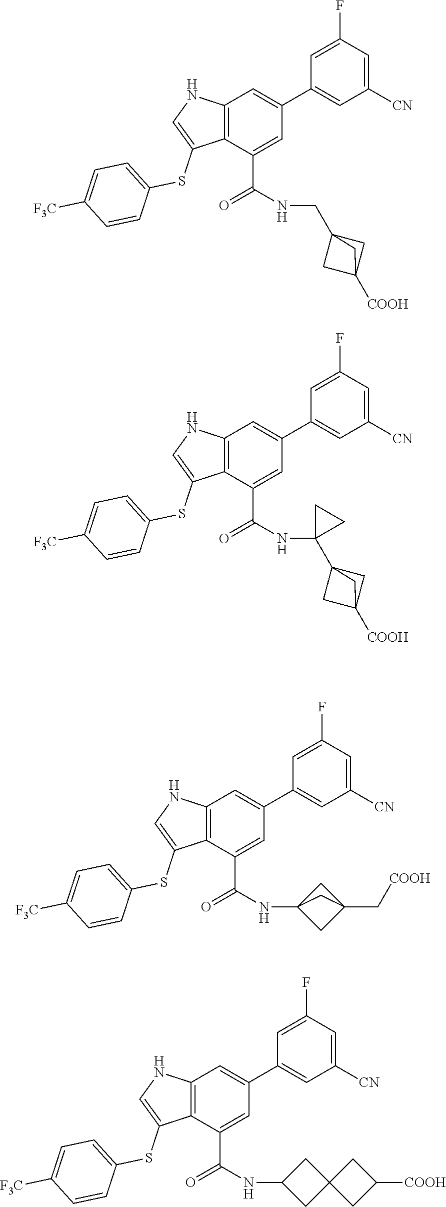 [SMILES: N#Cc1cc(F)cc(-c2cc(C(=O)NCC34CC(C(=O)O)(C3)C4)c3c(Sc4ccc(C(F)(F)F)cc4)c[nH]c3c2)c1 ; N#Cc1cc(F)cc(-c2cc(C(=O)NC3(C45CC(C(=O)O)(C4)C5)CC3)c3c(Sc4ccc(C(F)(F)F)cc4)c[nH]c3c2)c1 ; N#Cc1cc(F)cc(-c2cc(C(=O)NC34CC(CC(=O)O)(C3)C4)c3c(Sc4ccc(C(F)(F)F)cc4)c[nH]c3c2)c1 ; N#Cc1cc(F)cc(-c2cc(C(=O)NC3CC4(C3)CC(C(=O)O)C4)c3c(Sc4ccc(C(F)(F)F)cc4)c[nH]c3c2)c1]

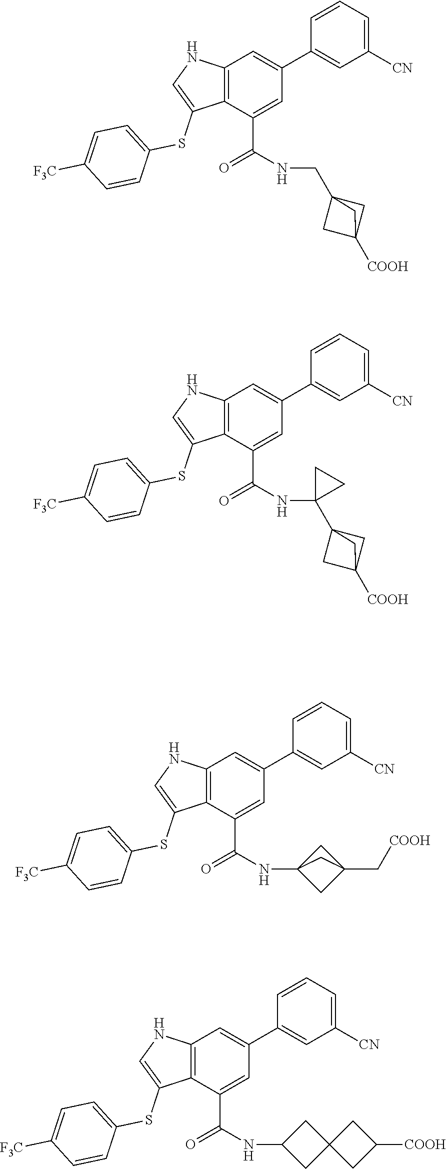 [SMILES: N#Cc1cccc(-c2cc(C(=O)NCC34CC(C(=O)O)(C3)C4)c3c(Sc4ccc(C(F)(F)F)cc4)c[nH]c3c2)c1 ; N#Cc1cccc(-c2cc(C(=O)NC3(C45CC(C(=O)O)(C4)C5)CC3)c3c(Sc4ccc(C(F)(F)F)cc4)c[nH]c3c2)c1 ; N#Cc1cccc(-c2cc(C(=O)NC34CC(CC(=O)O)(C3)C4)c3c(Sc4ccc(C(F)(F)F)cc4)c[nH]c3c2)c1 ; N#Cc1cccc(-c2cc(C(=O)NC3CC4(C3)CC(C(=O)O)C4)c3c(Sc4ccc(C(F)(F)F)cc4)c[nH]c3c2)c1]

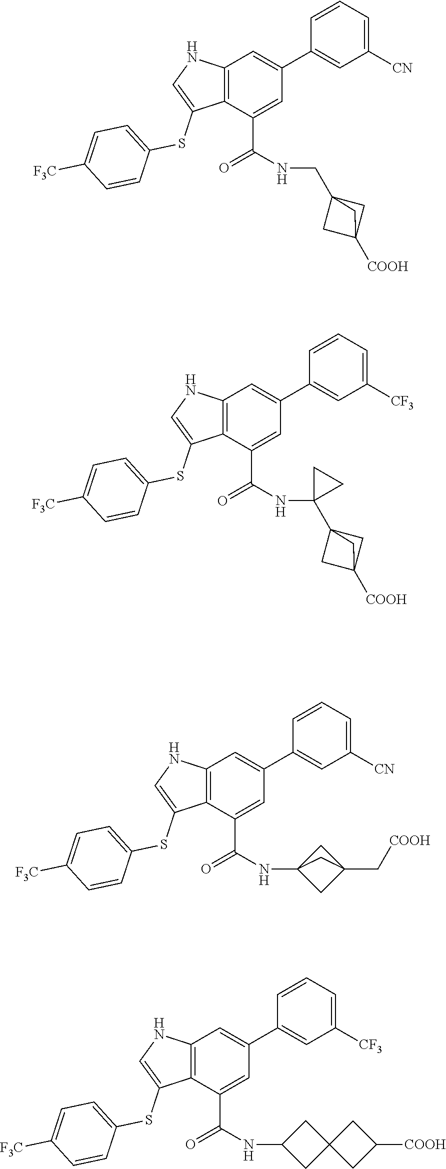 [SMILES: N#Cc1cccc(-c2cc(C(=O)NCC34CC(C(=O)O)(C3)C4)c3c(Sc4ccc(C(F)(F)F)cc4)c[nH]c3c2)c1 ; O=C(NC1(C23CC(C(=O)O)(C2)C3)CC1)c1cc(-c2cccc(C(F)(F)F)c2)cc2[nH]cc(Sc3ccc(C(F)(F)F)cc3)c12 ; N#Cc1cccc(-c2cc(C(=O)NC34CC(CC(=O)O)(C3)C4)c3c(Sc4ccc(C(F)(F)F)cc4)c[nH]c3c2)c1 ; O=C(NC1CC2(C1)CC(C(=O)O)C2)c1cc(-c2cccc(C(F)(F)F)c2)cc2[nH]cc(Sc3ccc(C(F)(F)F)cc3)c12]

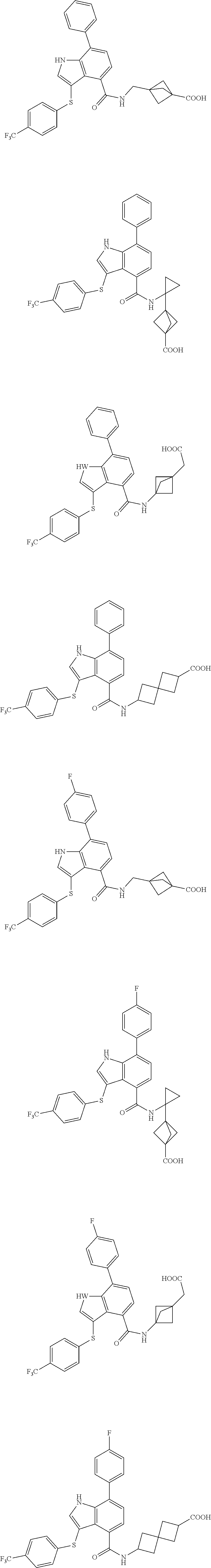

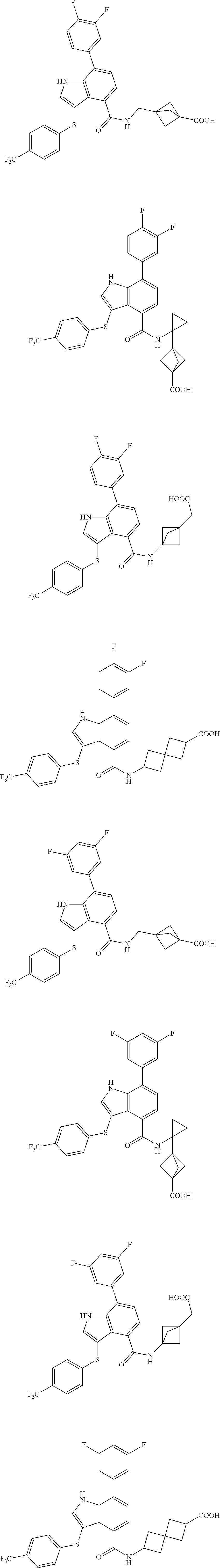

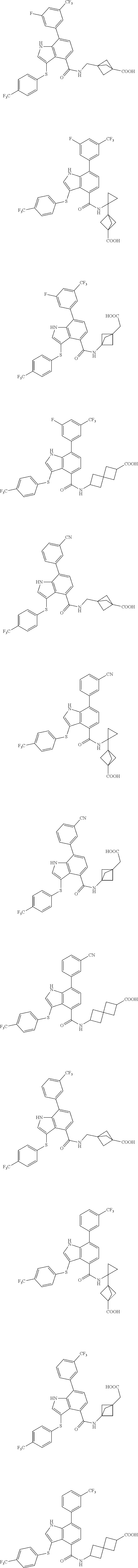

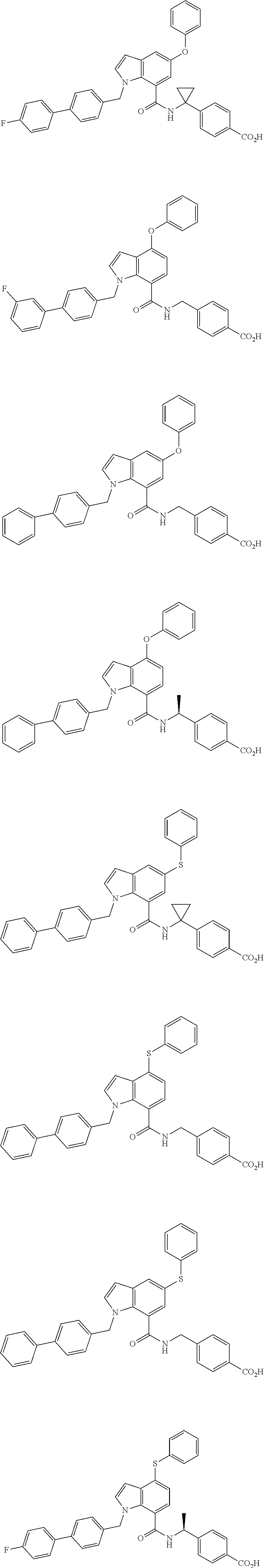

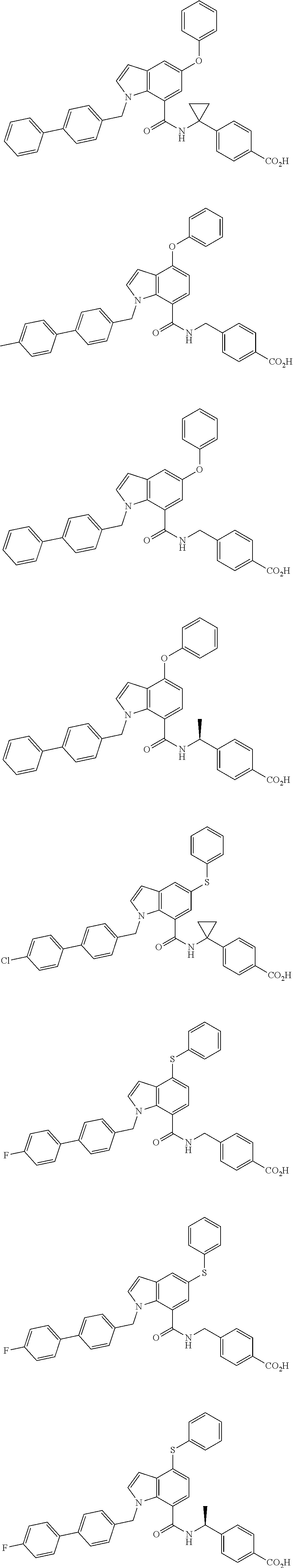

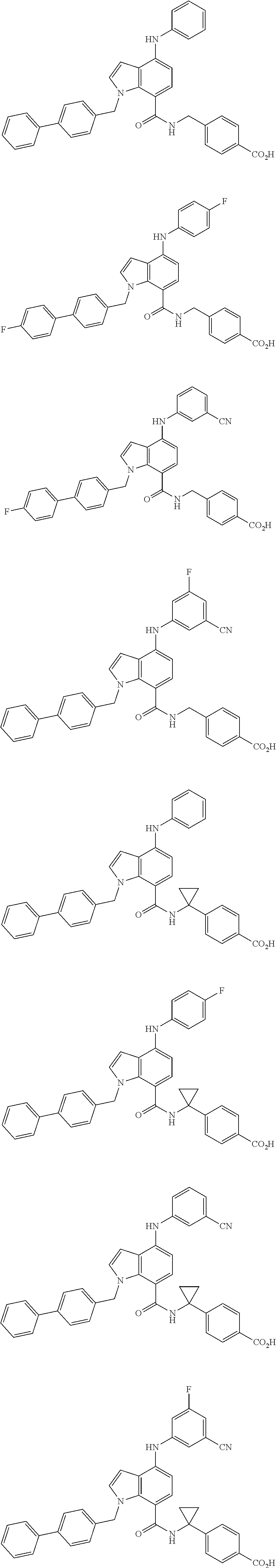

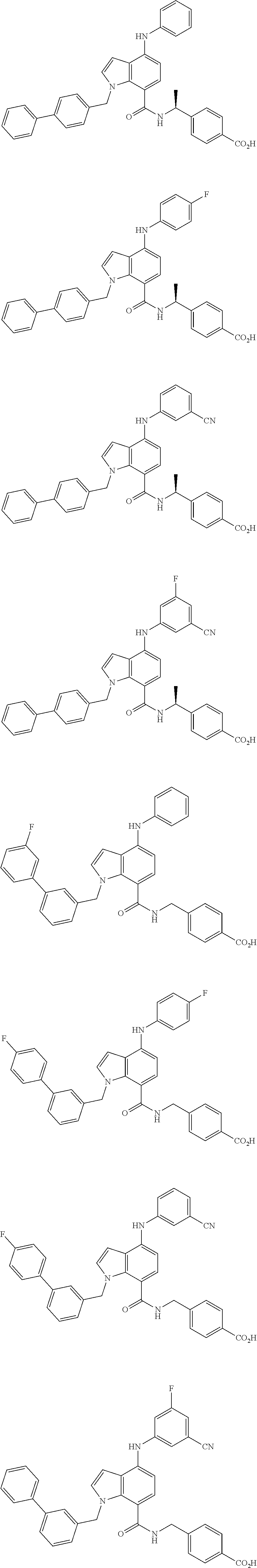

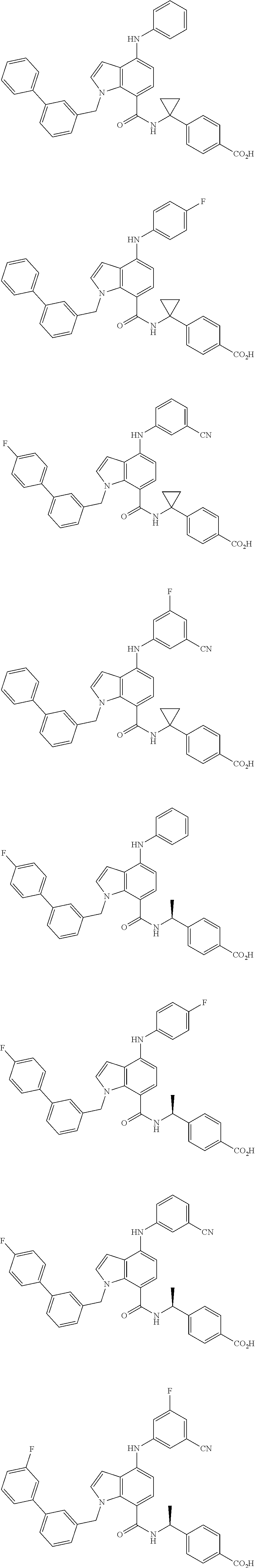

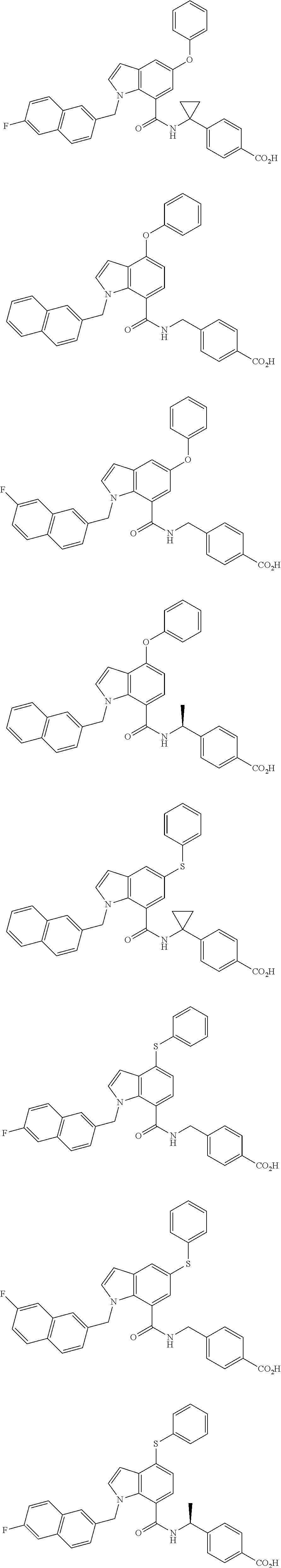

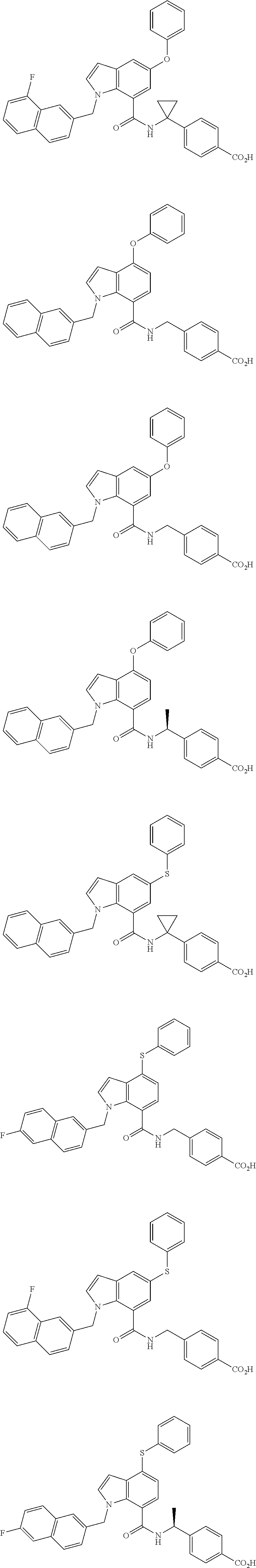

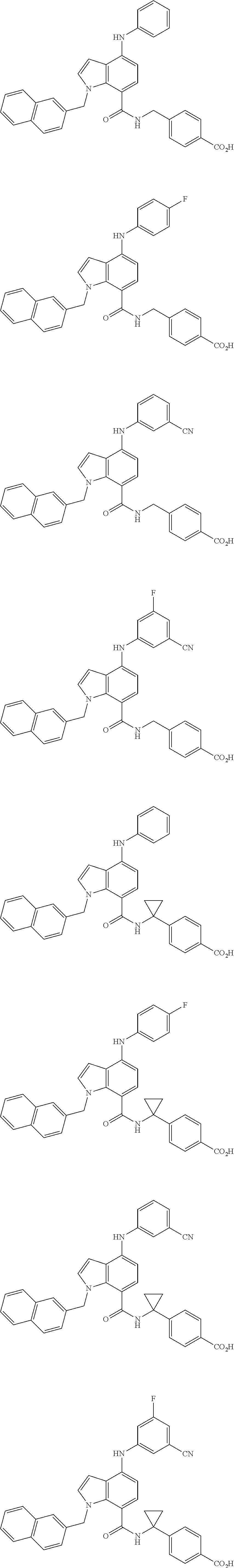

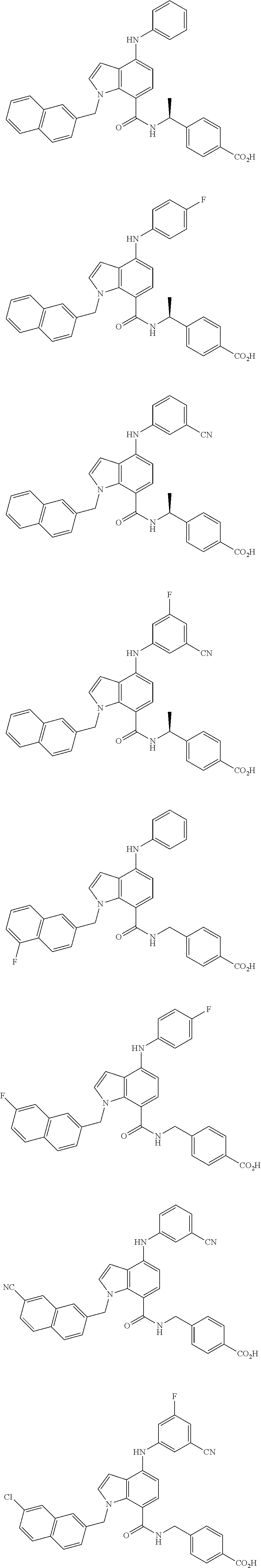

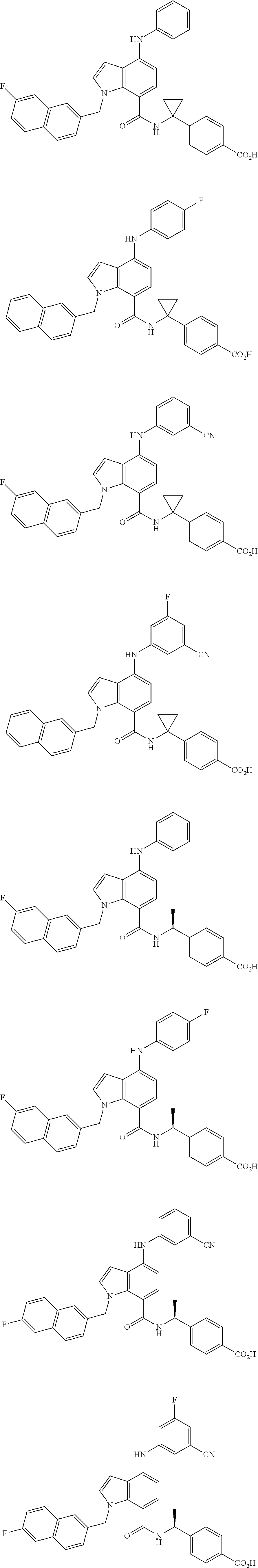

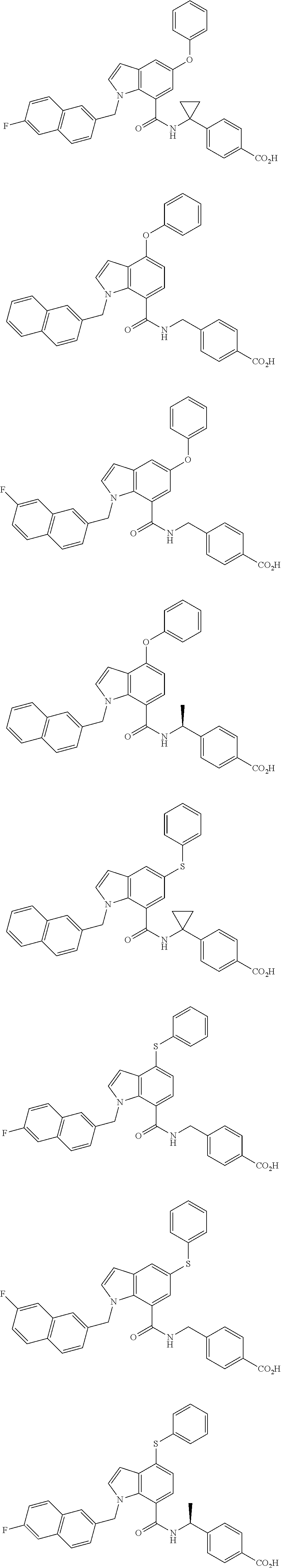

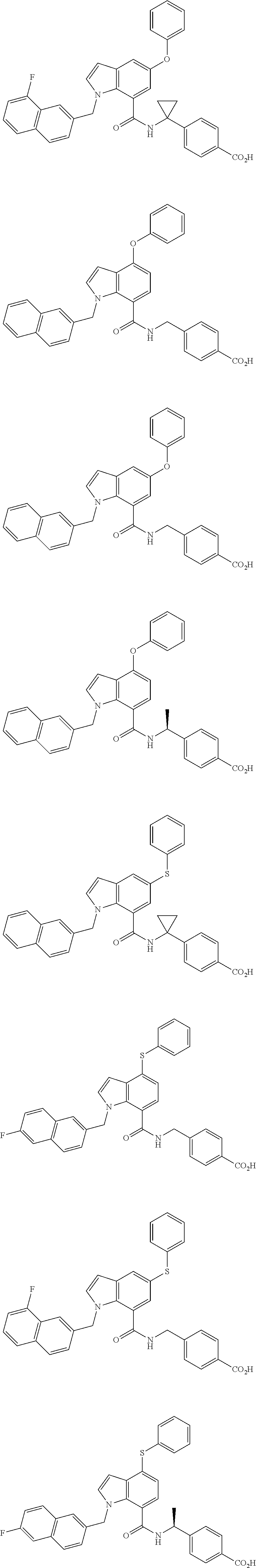

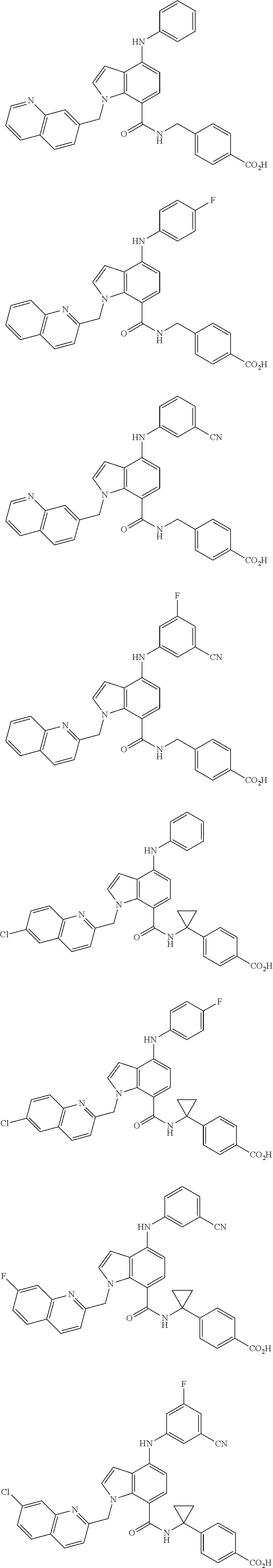

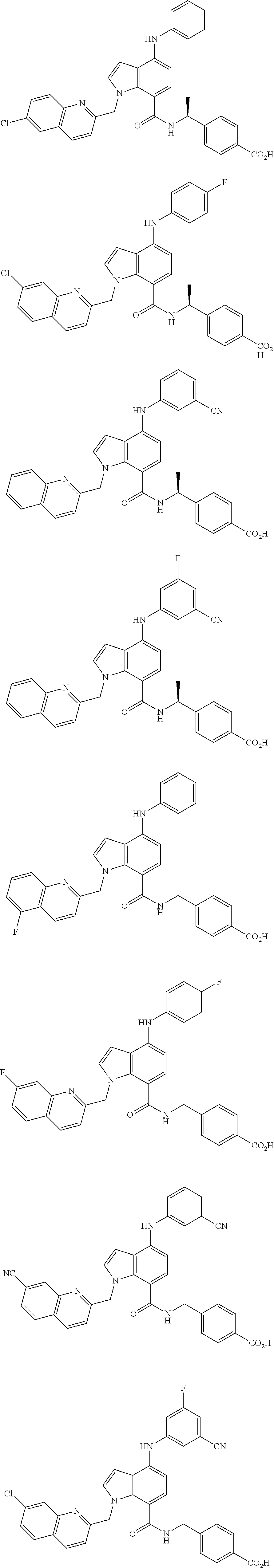

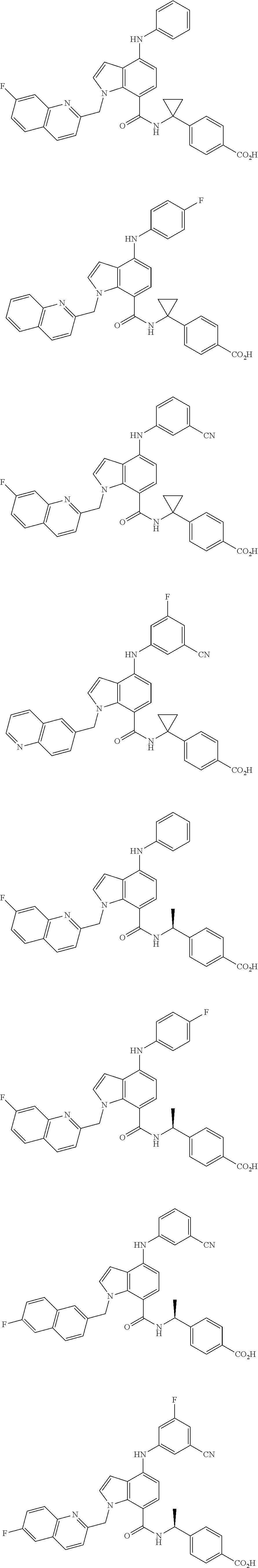

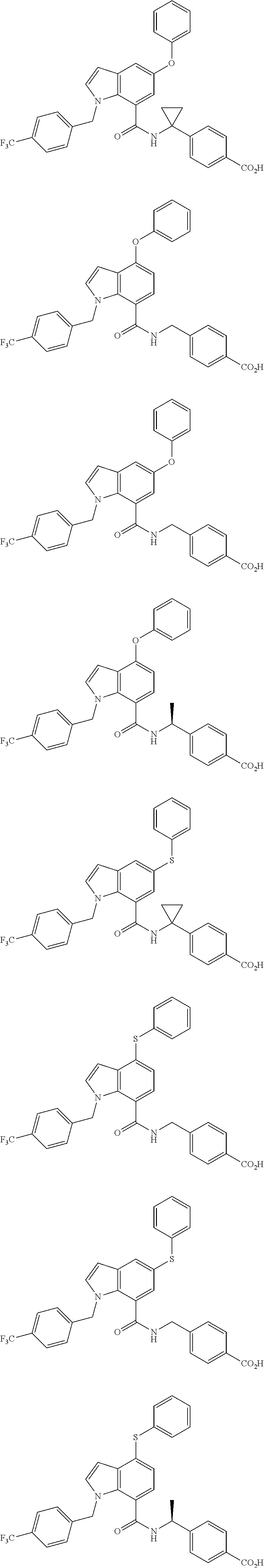

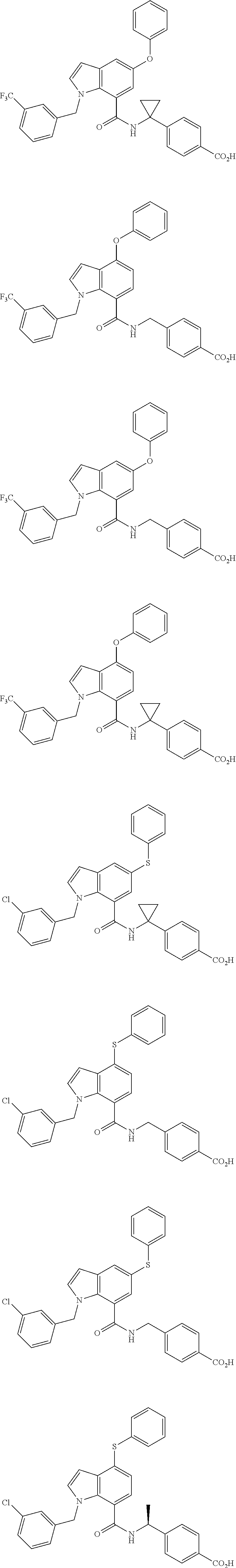

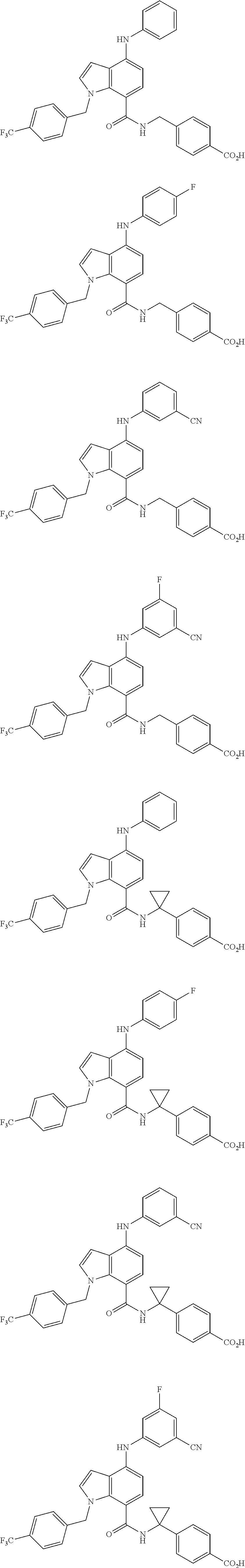

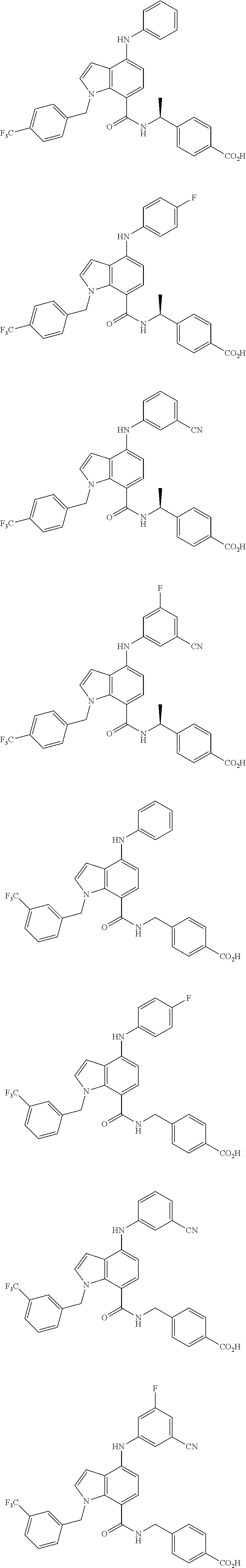

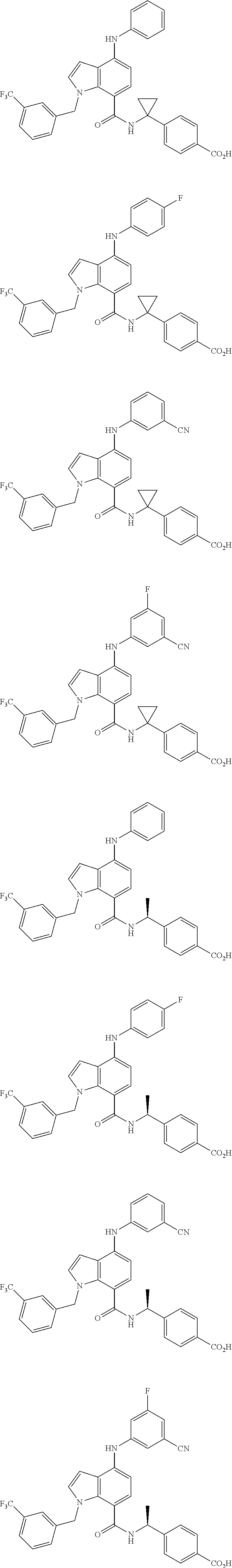

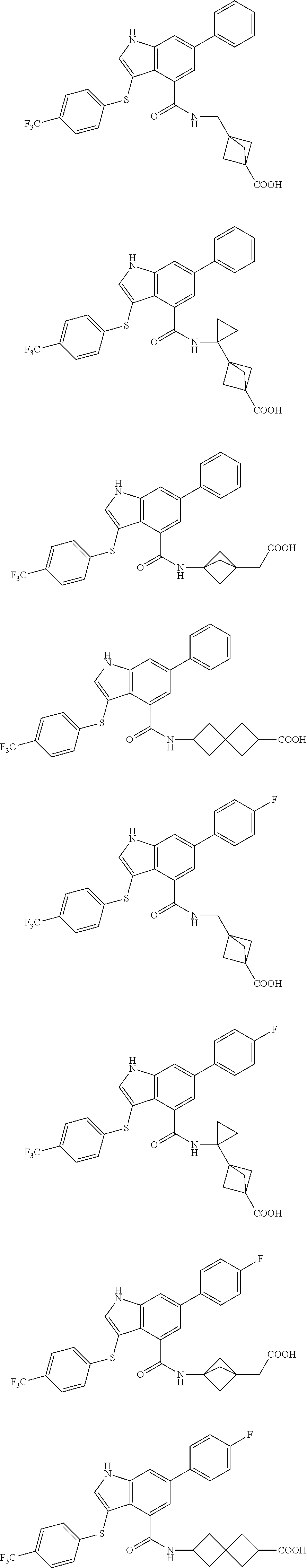

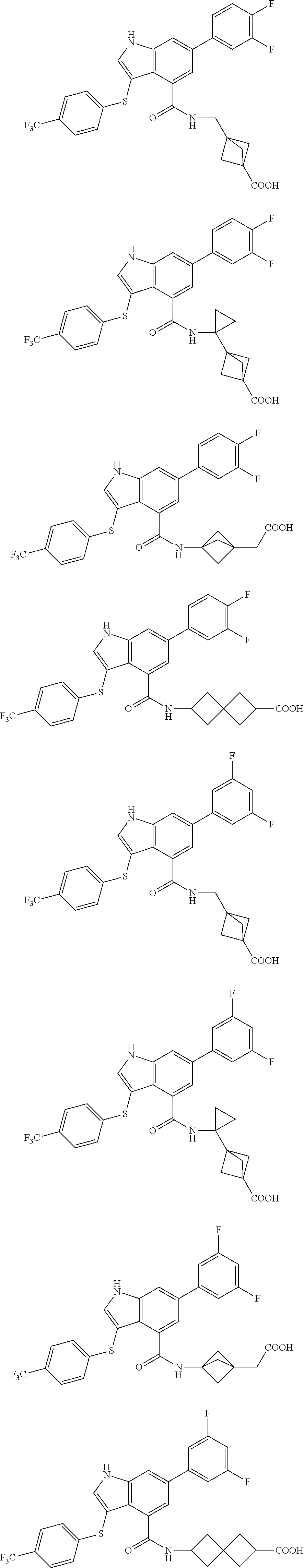

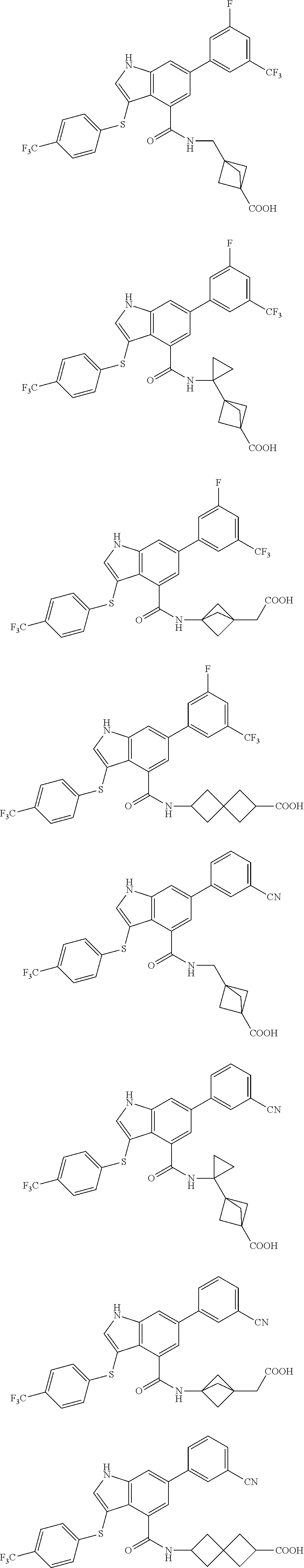

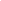

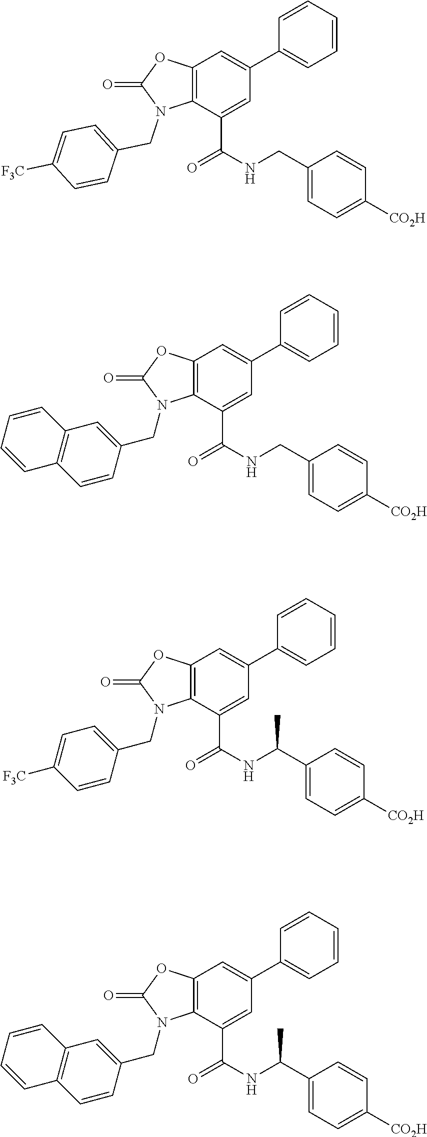 [SMILES: O=C(O)c1ccc(CNC(=O)c2cc(-c3ccccc3)cc3oc(=O)n(Cc4ccc(C(F)(F)F)cc4)c23)cc1 ; O=C(O)c1ccc(CNC(=O)c2cc(-c3ccccc3)cc3oc(=O)n(Cc4ccc5ccccc5c4)c23)cc1 ; C[C@H](NC(=O)c1cc(-c2ccccc2)cc2oc(=O)n(Cc3ccc(C(F)(F)F)cc3)c12)c1ccc(C(=O)O)cc1 ; C[C@H](NC(=O)c1cc(-c2ccccc2)cc2oc(=O)n(Cc3ccc4ccccc4c3)c12)c1ccc(C(=O)O)cc1]

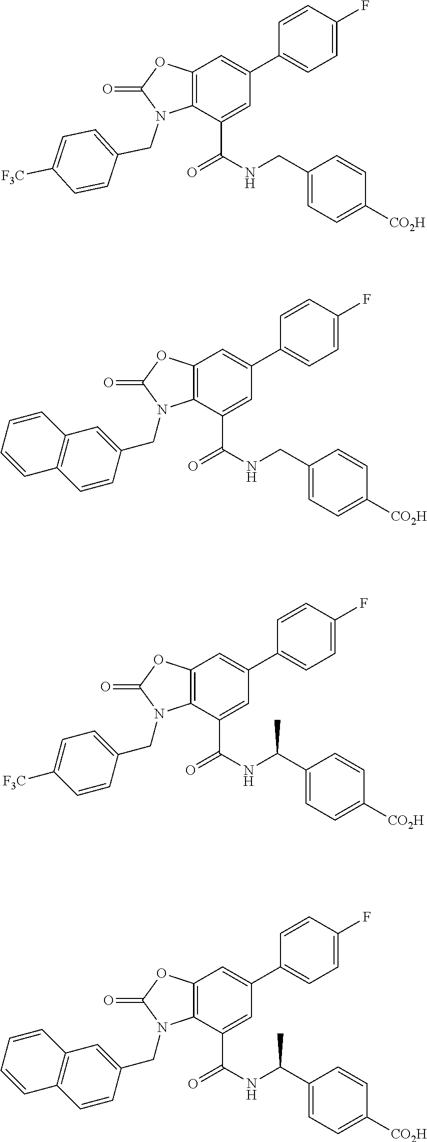 [SMILES: O=C(O)c1ccc(CNC(=O)c2cc(-c3ccc(F)cc3)cc3oc(=O)n(Cc4ccc(C(F)(F)F)cc4)c23)cc1 ; O=C(O)c1ccc(CNC(=O)c2cc(-c3ccc(F)cc3)cc3oc(=O)n(Cc4ccc5ccccc5c4)c23)cc1 ; C[C@H](NC(=O)c1cc(-c2ccc(F)cc2)cc2oc(=O)n(Cc3ccc(C(F)(F)F)cc3)c12)c1ccc(C(=O)O)cc1 ; C[C@H](NC(=O)c1cc(-c2ccc(F)cc2)cc2oc(=O)n(Cc3ccc4ccccc4c3)c12)c1ccc(C(=O)O)cc1]

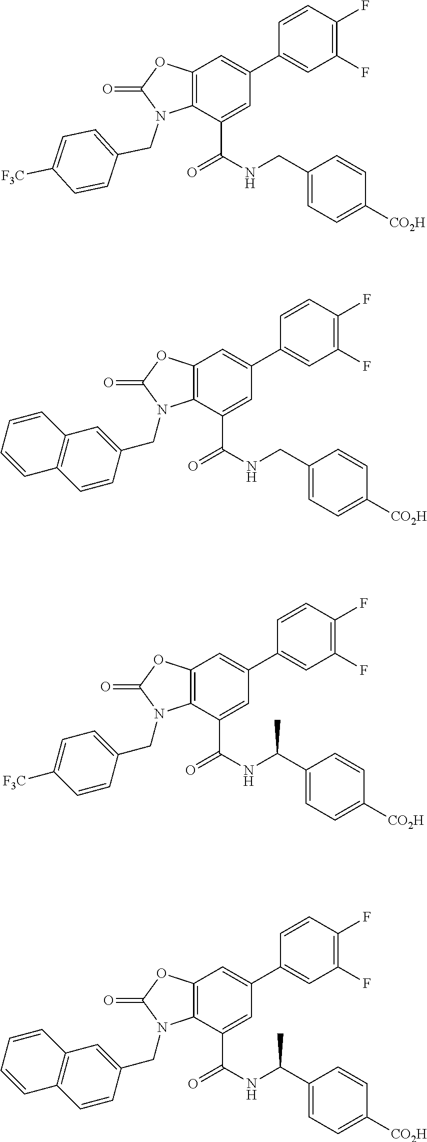 [SMILES: O=C(O)c1ccc(CNC(=O)c2cc(-c3ccc(F)c(F)c3)cc3oc(=O)n(Cc4ccc(C(F)(F)F)cc4)c23)cc1 ; O=C(O)c1ccc(CNC(=O)c2cc(-c3ccc(F)c(F)c3)cc3oc(=O)n(Cc4ccc5ccccc5c4)c23)cc1 ; C[C@H](NC(=O)c1cc(-c2ccc(F)c(F)c2)cc2oc(=O)n(Cc3ccc(C(F)(F)F)cc3)c12)c1ccc(C(=O)O)cc1 ; C[C@H](NC(=O)c1cc(-c2ccc(F)c(F)c2)cc2oc(=O)n(Cc3ccc4ccccc4c3)c12)c1ccc(C(=O)O)cc1]

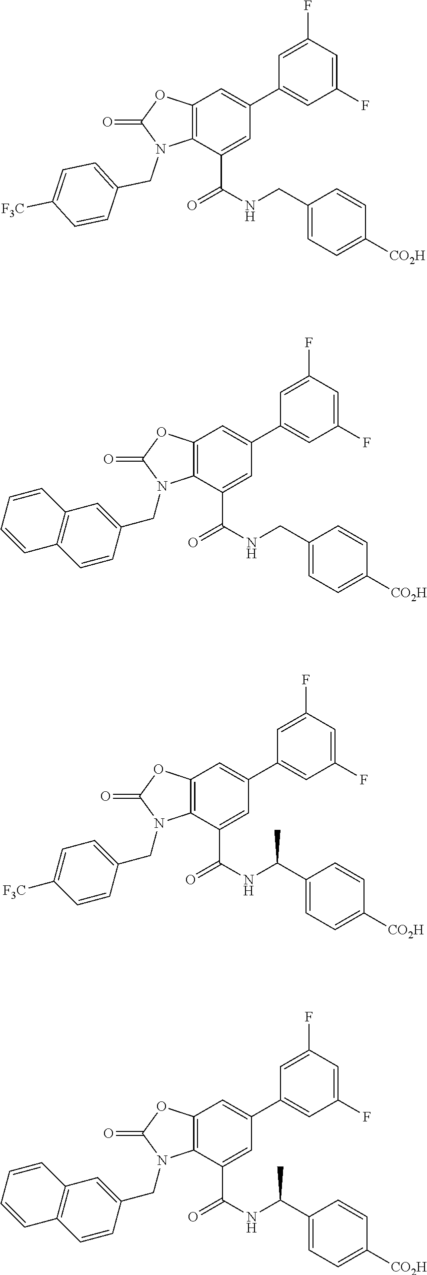 [SMILES: O=C(O)c1ccc(CNC(=O)c2cc(-c3cc(F)cc(F)c3)cc3oc(=O)n(Cc4ccc(C(F)(F)F)cc4)c23)cc1 ; O=C(O)c1ccc(CNC(=O)c2cc(-c3cc(F)cc(F)c3)cc3oc(=O)n(Cc4ccc5ccccc5c4)c23)cc1 ; C[C@H](NC(=O)c1cc(-c2cc(F)cc(F)c2)cc2oc(=O)n(Cc3ccc(C(F)(F)F)cc3)c12)c1ccc(C(=O)O)cc1 ; C[C@H](NC(=O)c1cc(-c2cc(F)cc(F)c2)cc2oc(=O)n(Cc3ccc4ccccc4c3)c12)c1ccc(C(=O)O)cc1]

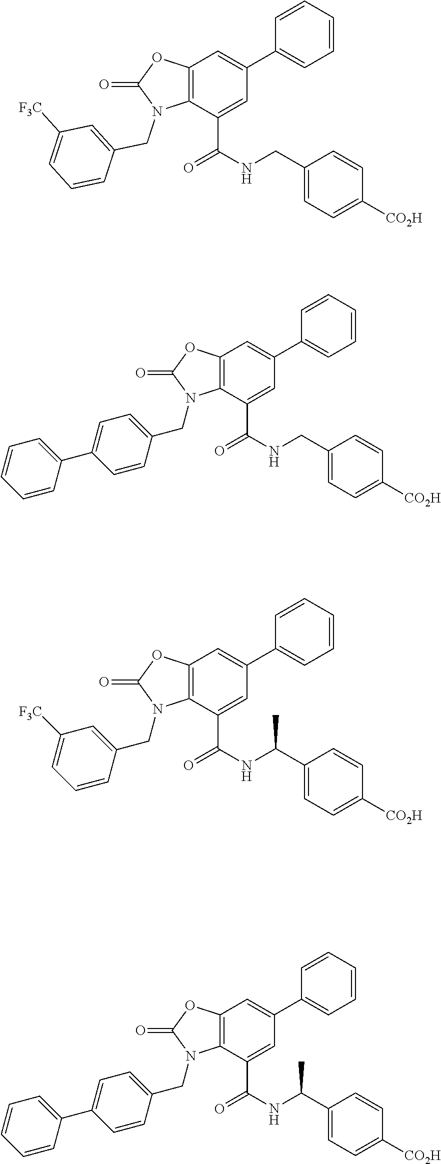 [SMILES: O=C(O)c1ccc(CNC(=O)c2cc(-c3ccccc3)cc3oc(=O)n(Cc4cccc(C(F)(F)F)c4)c23)cc1 ; O=C(O)c1ccc(CNC(=O)c2cc(-c3ccccc3)cc3oc(=O)n(Cc4ccc(-c5ccccc5)cc4)c23)cc1 ; C[C@H](NC(=O)c1cc(-c2ccccc2)cc2oc(=O)n(Cc3cccc(C(F)(F)F)c3)c12)c1ccc(C(=O)O)cc1 ; C[C@H](NC(=O)c1cc(-c2ccccc2)cc2oc(=O)n(Cc3ccc(-c4ccccc4)cc3)c12)c1ccc(C(=O)O)cc1]

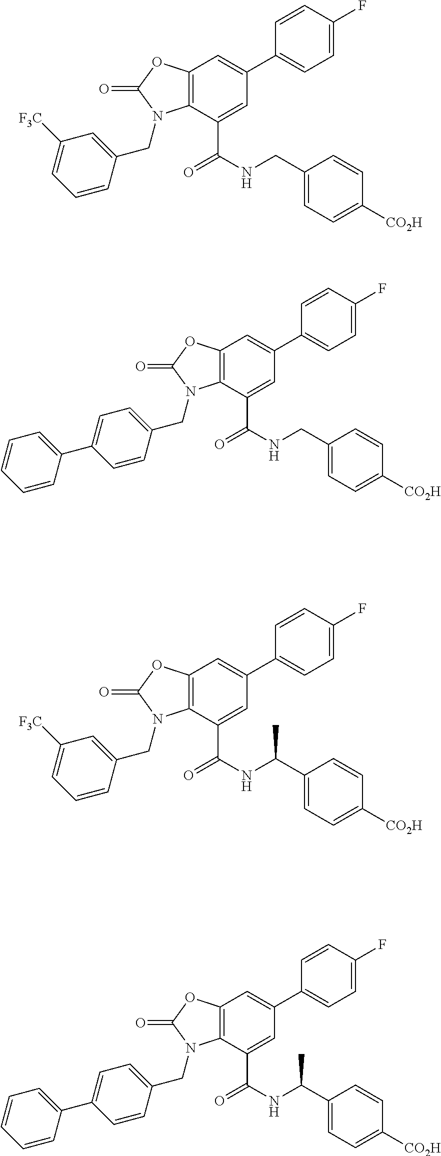 [SMILES: O=C(O)c1ccc(CNC(=O)c2cc(-c3ccc(F)cc3)cc3oc(=O)n(Cc4cccc(C(F)(F)F)c4)c23)cc1 ; O=C(O)c1ccc(CNC(=O)c2cc(-c3ccc(F)cc3)cc3oc(=O)n(Cc4ccc(-c5ccccc5)cc4)c23)cc1 ; C[C@H](NC(=O)c1cc(-c2ccc(F)cc2)cc2oc(=O)n(Cc3cccc(C(F)(F)F)c3)c12)c1ccc(C(=O)O)cc1 ; C[C@H](NC(=O)c1cc(-c2ccc(F)cc2)cc2oc(=O)n(Cc3ccc(-c4ccccc4)cc3)c12)c1ccc(C(=O)O)cc1]

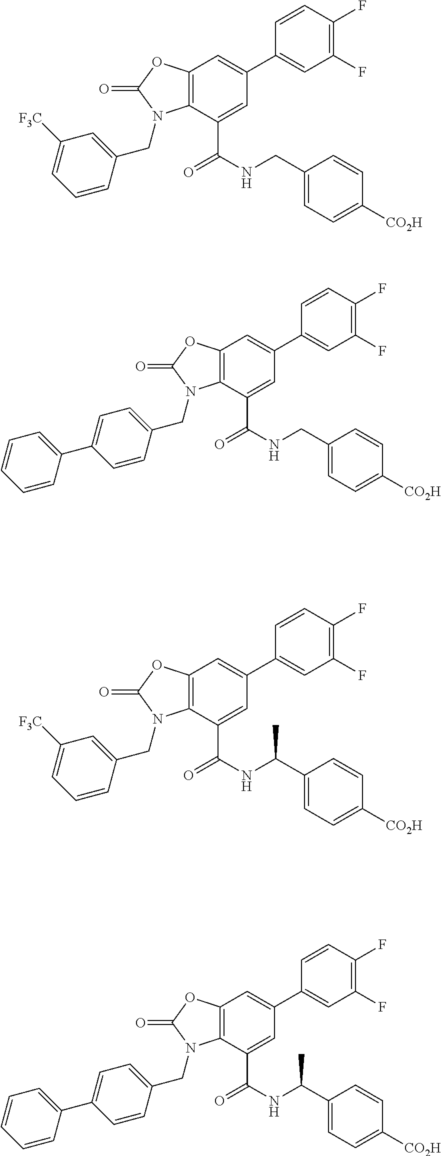 [SMILES: O=C(O)c1ccc(CNC(=O)c2cc(-c3ccc(F)c(F)c3)cc3oc(=O)n(Cc4cccc(C(F)(F)F)c4)c23)cc1 ; O=C(O)c1ccc(CNC(=O)c2cc(-c3ccc(F)c(F)c3)cc3oc(=O)n(Cc4ccc(-c5ccccc5)cc4)c23)cc1 ; C[C@H](NC(=O)c1cc(-c2ccc(F)c(F)c2)cc2oc(=O)n(Cc3cccc(C(F)(F)F)c3)c12)c1ccc(C(=O)O)cc1 ; C[C@H](NC(=O)c1cc(-c2ccc(F)c(F)c2)cc2oc(=O)n(Cc3ccc(-c4ccccc4)cc3)c12)c1ccc(C(=O)O)cc1]

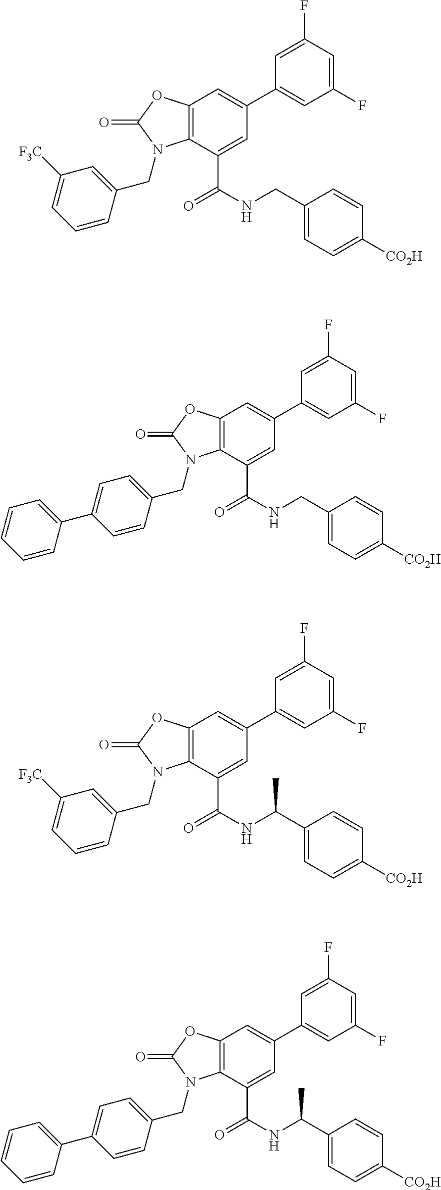 [SMILES: O=C(O)c1ccc(CNC(=O)c2cc(-c3cc(F)cc(F)c3)cc3oc(=O)n(Cc4cccc(C(F)(F)F)c4)c23)cc1 ; O=C(O)c1ccc(CNC(=O)c2cc(-c3cc(F)cc(F)c3)cc3oc(=O)n(Cc4ccc(-c5ccccc5)cc4)c23)cc1 ; C[C@H](NC(=O)c1cc(-c2cc(F)cc(F)c2)cc2oc(=O)n(Cc3cccc(C(F)(F)F)c3)c12)c1ccc(C(=O)O)cc1 ; C[C@H](NC(=O)c1cc(-c2cc(F)cc(F)c2)cc2oc(=O)n(Cc3ccc(-c4ccccc4)cc3)c12)c1ccc(C(=O)O)cc1]

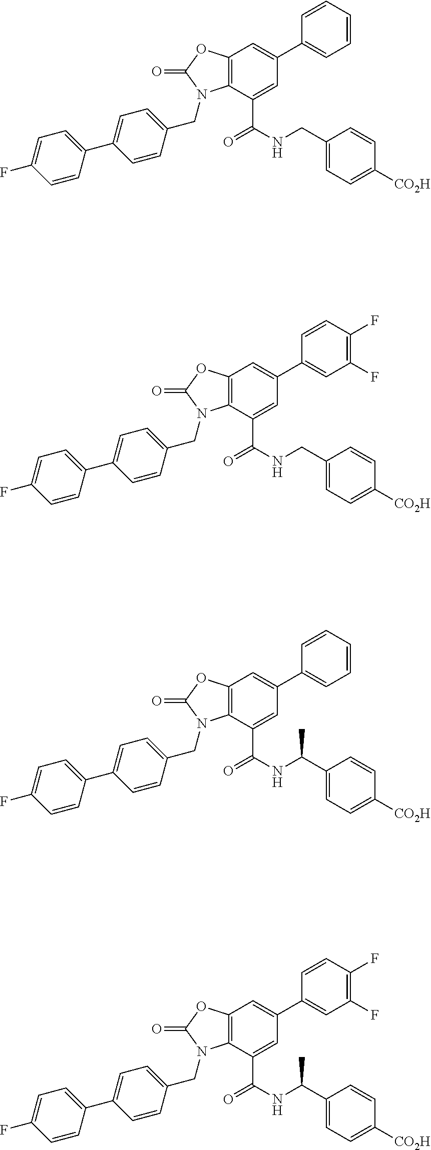 [SMILES: O=C(O)c1ccc(CNC(=O)c2cc(-c3ccccc3)cc3oc(=O)n(Cc4ccc(-c5ccc(F)cc5)cc4)c23)cc1 ; O=C(O)c1ccc(CNC(=O)c2cc(-c3ccc(F)c(F)c3)cc3oc(=O)n(Cc4ccc(-c5ccc(F)cc5)cc4)c23)cc1 ; C[C@H](NC(=O)c1cc(-c2ccccc2)cc2oc(=O)n(Cc3ccc(-c4ccc(F)cc4)cc3)c12)c1ccc(C(=O)O)cc1 ; C[C@H](NC(=O)c1cc(-c2ccc(F)c(F)c2)cc2oc(=O)n(Cc3ccc(-c4ccc(F)cc4)cc3)c12)c1ccc(C(=O)O)cc1]

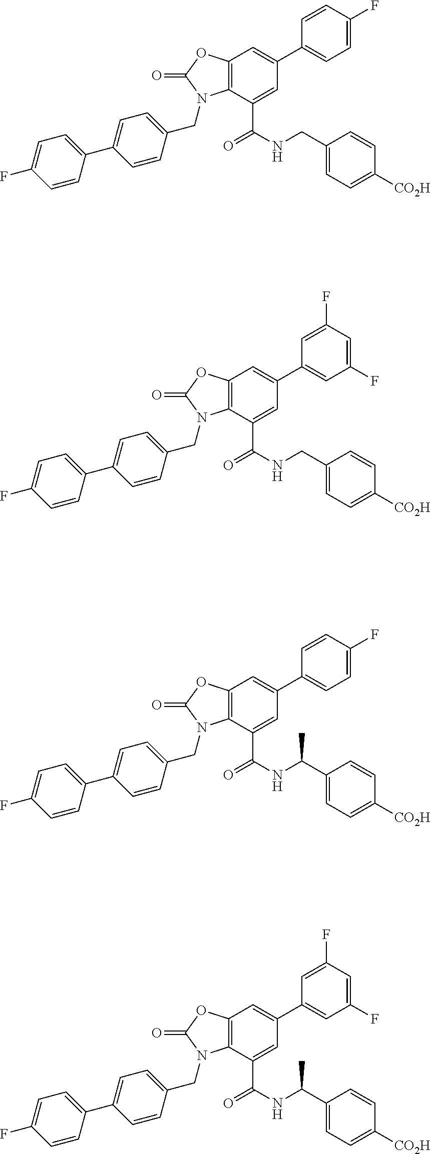 [SMILES: O=C(O)c1ccc(CNC(=O)c2cc(-c3ccc(F)cc3)cc3oc(=O)n(Cc4ccc(-c5ccc(F)cc5)cc4)c23)cc1 ; O=C(O)c1ccc(CNC(=O)c2cc(-c3cc(F)cc(F)c3)cc3oc(=O)n(Cc4ccc(-c5ccc(F)cc5)cc4)c23)cc1 ; C[C@H](NC(=O)c1cc(-c2ccc(F)cc2)cc2oc(=O)n(Cc3ccc(-c4ccc(F)cc4)cc3)c12)c1ccc(C(=O)O)cc1 ; C[C@H](NC(=O)c1cc(-c2cc(F)cc(F)c2)cc2oc(=O)n(Cc3ccc(-c4ccc(F)cc4)cc3)c12)c1ccc(C(=O)O)cc1]

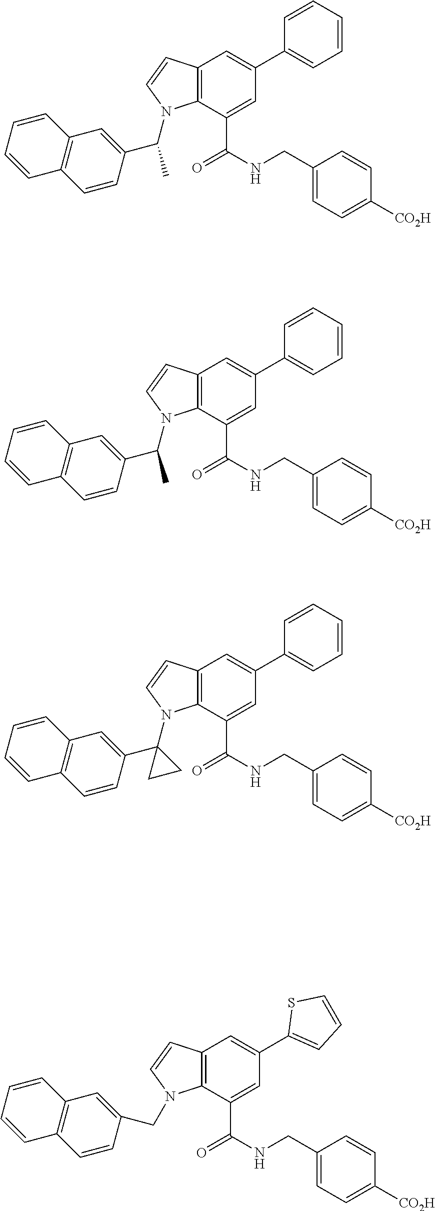 [SMILES: C[C@H](c1ccc2ccccc2c1)n1ccc2cc(-c3ccccc3)cc(C(=O)NCc3ccc(C(=O)O)cc3)c21 ; C[C@@H](c1ccc2ccccc2c1)n1ccc2cc(-c3ccccc3)cc(C(=O)NCc3ccc(C(=O)O)cc3)c21 ; O=C(O)c1ccc(CNC(=O)c2cc(-c3ccccc3)cc3ccn(C4(c5ccc6ccccc6c5)CC4)c23)cc1 ; O=C(O)c1ccc(CNC(=O)c2cc(-c3cccs3)cc3ccn(Cc4ccc5ccccc5c4)c23)cc1]

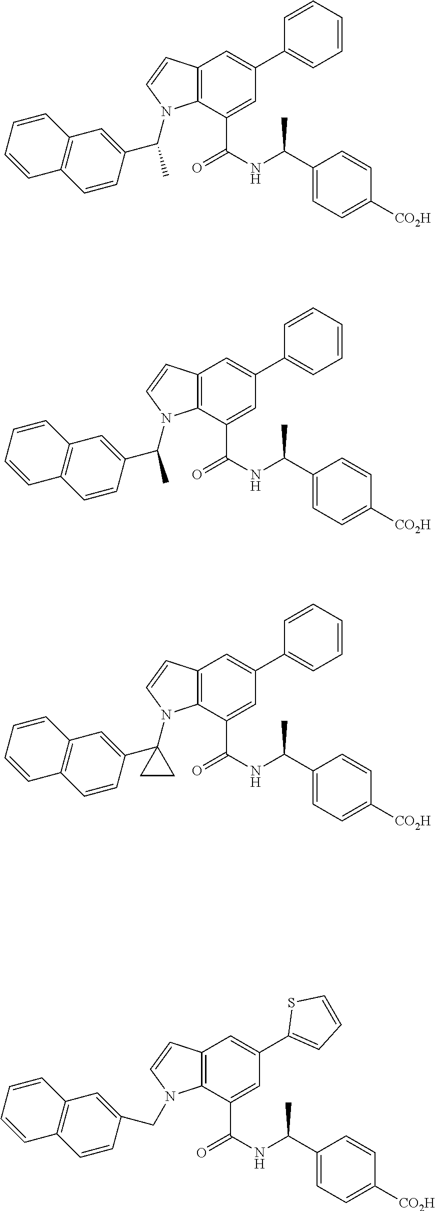 [SMILES: C[C@H](NC(=O)c1cc(-c2ccccc2)cc2ccn([C@H](C)c3ccc4ccccc4c3)c12)c1ccc(C(=O)O)cc1 ; C[C@H](NC(=O)c1cc(-c2ccccc2)cc2ccn([C@@H](C)c3ccc4ccccc4c3)c12)c1ccc(C(=O)O)cc1 ; C[C@H](NC(=O)c1cc(-c2ccccc2)cc2ccn(C3(c4ccc5ccccc5c4)CC3)c12)c1ccc(C(=O)O)cc1 ; C[C@H](NC(=O)c1cc(-c2cccs2)cc2ccn(Cc3ccc4ccccc4c3)c12)c1ccc(C(=O)O)cc1]

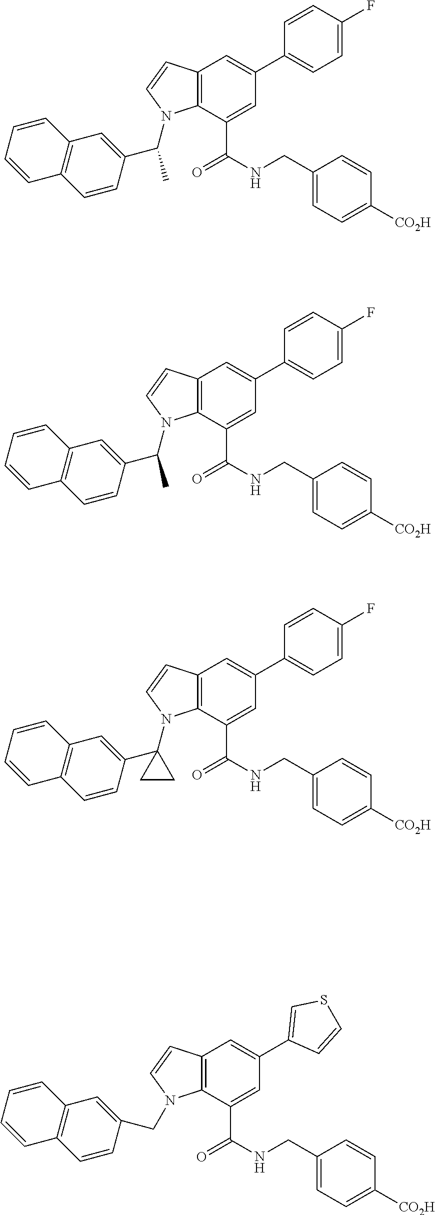 [SMILES: C[C@H](c1ccc2ccccc2c1)n1ccc2cc(-c3ccc(F)cc3)cc(C(=O)NCc3ccc(C(=O)O)cc3)c21 ; C[C@@H](c1ccc2ccccc2c1)n1ccc2cc(-c3ccc(F)cc3)cc(C(=O)NCc3ccc(C(=O)O)cc3)c21 ; O=C(O)c1ccc(CNC(=O)c2cc(-c3ccc(F)cc3)cc3ccn(C4(c5ccc6ccccc6c5)CC4)c23)cc1 ; O=C(O)c1ccc(CNC(=O)c2cc(-c3ccsc3)cc3ccn(Cc4ccc5ccccc5c4)c23)cc1]

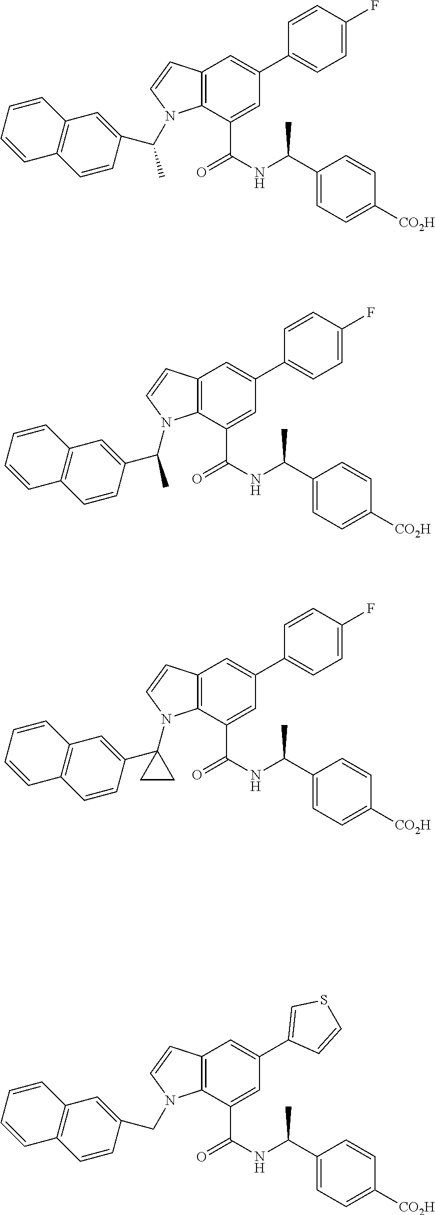 [SMILES: C[C@H](NC(=O)c1cc(-c2ccc(F)cc2)cc2ccn([C@H](C)c3ccc4ccccc4c3)c12)c1ccc(C(=O)O)cc1 ; C[C@H](NC(=O)c1cc(-c2ccc(F)cc2)cc2ccn([C@@H](C)c3ccc4ccccc4c3)c12)c1ccc(C(=O)O)cc1 ; C[C@H](NC(=O)c1cc(-c2ccc(F)cc2)cc2ccn(C3(c4ccc5ccccc5c4)CC3)c12)c1ccc(C(=O)O)cc1 ; C[C@H](NC(=O)c1cc(-c2ccsc2)cc2ccn(Cc3ccc4ccccc4c3)c12)c1ccc(C(=O)O)cc1]

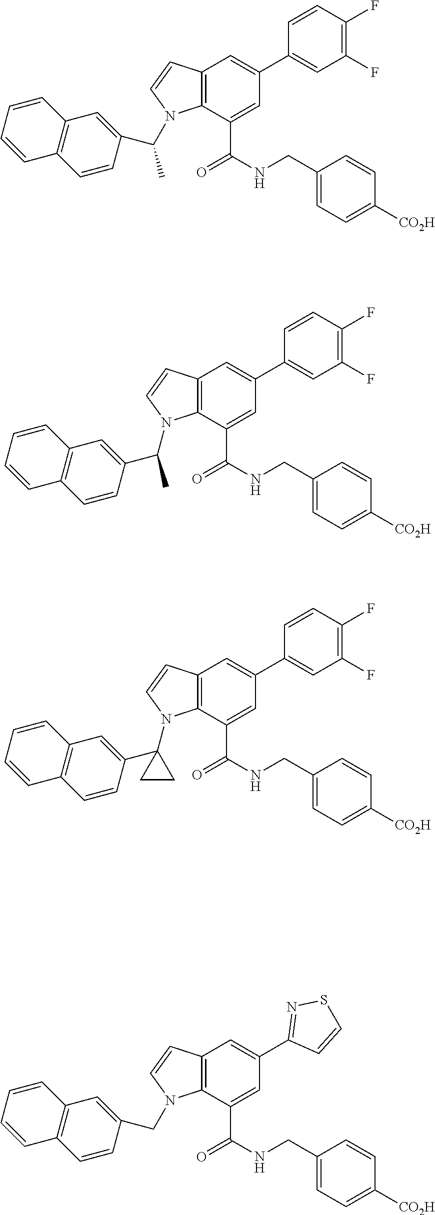 [SMILES: C[C@H](c1ccc2ccccc2c1)n1ccc2cc(-c3ccc(F)c(F)c3)cc(C(=O)NCc3ccc(C(=O)O)cc3)c21 ; C[C@@H](c1ccc2ccccc2c1)n1ccc2cc(-c3ccc(F)c(F)c3)cc(C(=O)NCc3ccc(C(=O)O)cc3)c21 ; O=C(O)c1ccc(CNC(=O)c2cc(-c3ccc(F)c(F)c3)cc3ccn(C4(c5ccc6ccccc6c5)CC4)c23)cc1 ; O=C(O)c1ccc(CNC(=O)c2cc(-c3ccsn3)cc3ccn(Cc4ccc5ccccc5c4)c23)cc1]

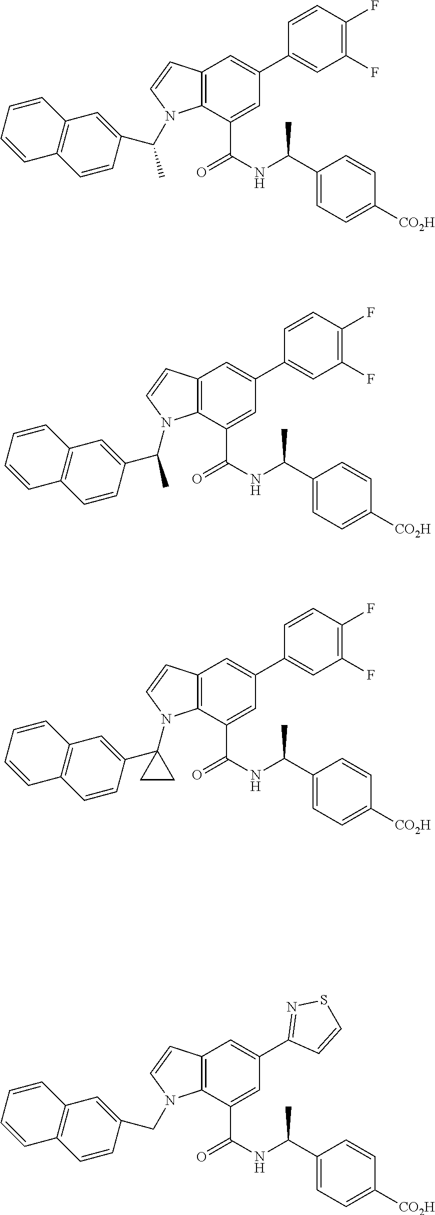 [SMILES: C[C@H](NC(=O)c1cc(-c2ccc(F)c(F)c2)cc2ccn([C@H](C)c3ccc4ccccc4c3)c12)c1ccc(C(=O)O)cc1 ; C[C@H](NC(=O)c1cc(-c2ccc(F)c(F)c2)cc2ccn([C@@H](C)c3ccc4ccccc4c3)c12)c1ccc(C(=O)O)cc1 ; C[C@H](NC(=O)c1cc(-c2ccc(F)c(F)c2)cc2ccn(C3(c4ccc5ccccc5c4)CC3)c12)c1ccc(C(=O)O)cc1 ; C[C@H](NC(=O)c1cc(-c2ccsn2)cc2ccn(Cc3ccc4ccccc4c3)c12)c1ccc(C(=O)O)cc1]

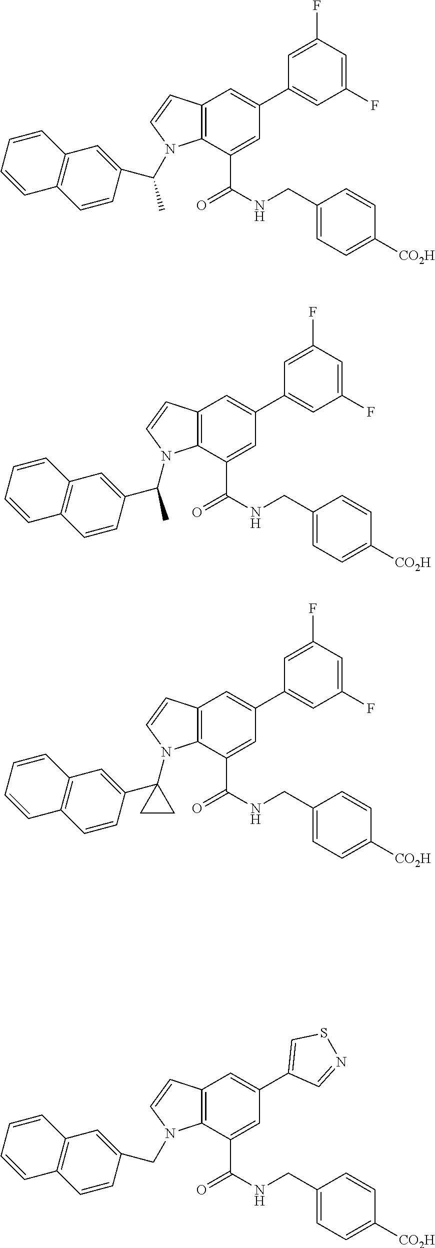 [SMILES: C[C@H](c1ccc2ccccc2c1)n1ccc2cc(-c3cc(F)cc(F)c3)cc(C(=O)NCc3ccc(C(=O)O)cc3)c21 ; C[C@@H](c1ccc2ccccc2c1)n1ccc2cc(-c3cc(F)cc(F)c3)cc(C(=O)NCc3ccc(C(=O)O)cc3)c21 ; O=C(O)c1ccc(CNC(=O)c2cc(-c3cc(F)cc(F)c3)cc3ccn(C4(c5ccc6ccccc6c5)CC4)c23)cc1 ; O=C(O)c1ccc(CNC(=O)c2cc(-c3cnsc3)cc3ccn(Cc4ccc5ccccc5c4)c23)cc1]

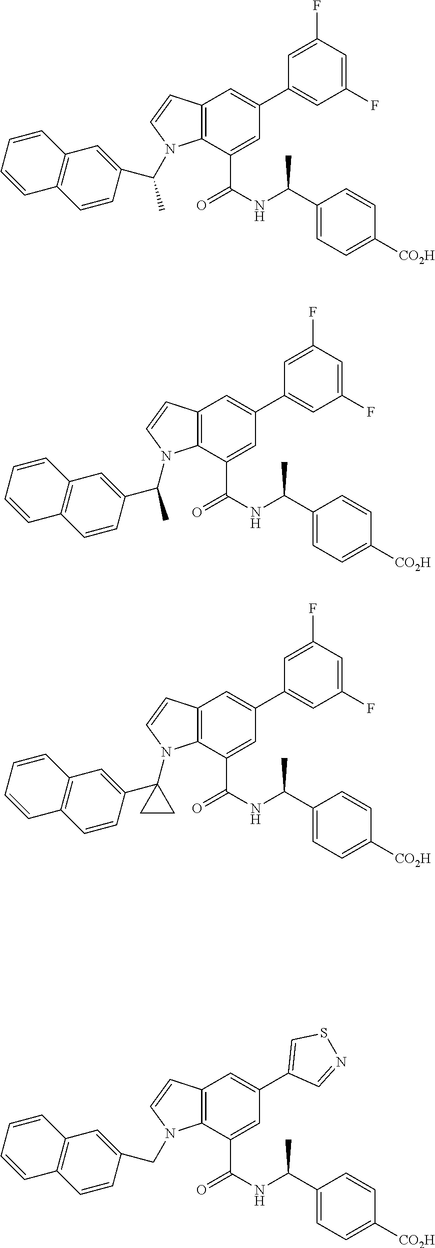 [SMILES: C[C@H](NC(=O)c1cc(-c2cc(F)cc(F)c2)cc2ccn([C@H](C)c3ccc4ccccc4c3)c12)c1ccc(C(=O)O)cc1 ; C[C@H](NC(=O)c1cc(-c2cc(F)cc(F)c2)cc2ccn([C@@H](C)c3ccc4ccccc4c3)c12)c1ccc(C(=O)O)cc1 ; C[C@H](NC(=O)c1cc(-c2cc(F)cc(F)c2)cc2ccn(C3(c4ccc5ccccc5c4)CC3)c12)c1ccc(C(=O)O)cc1 ; C[C@H](NC(=O)c1cc(-c2cnsc2)cc2ccn(Cc3ccc4ccccc4c3)c12)c1ccc(C(=O)O)cc1]

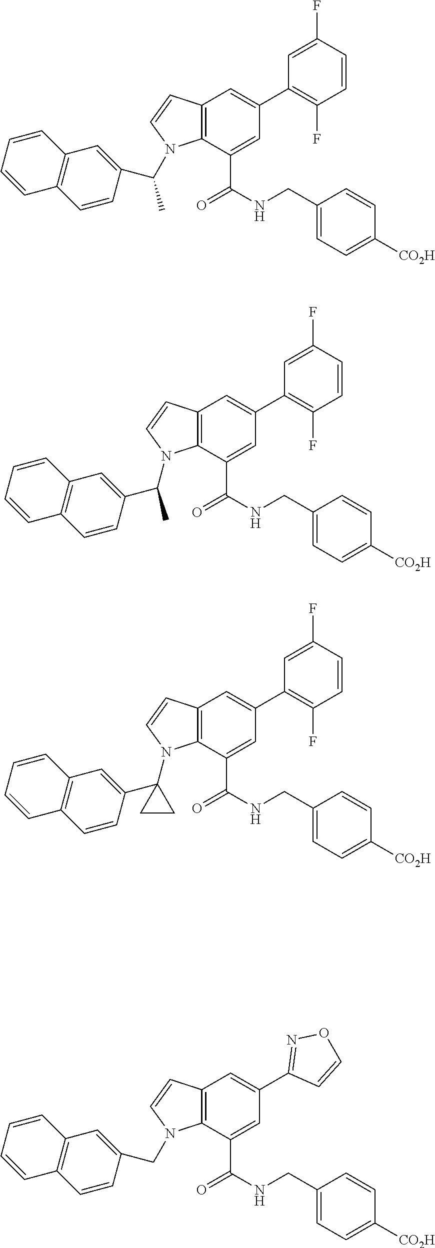 [SMILES: C[C@H](c1ccc2ccccc2c1)n1ccc2cc(-c3cc(F)ccc3F)cc(C(=O)NCc3ccc(C(=O)O)cc3)c21 ; C[C@@H](c1ccc2ccccc2c1)n1ccc2cc(-c3cc(F)ccc3F)cc(C(=O)NCc3ccc(C(=O)O)cc3)c21 ; O=C(O)c1ccc(CNC(=O)c2cc(-c3cc(F)ccc3F)cc3ccn(C4(c5ccc6ccccc6c5)CC4)c23)cc1 ; O=C(O)c1ccc(CNC(=O)c2cc(-c3ccon3)cc3ccn(Cc4ccc5ccccc5c4)c23)cc1]

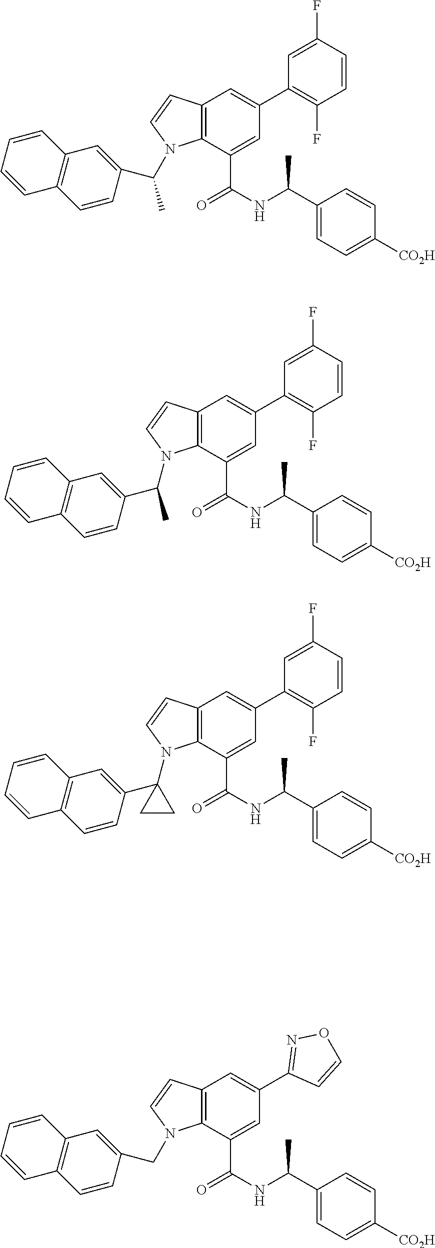 [SMILES: C[C@H](NC(=O)c1cc(-c2cc(F)ccc2F)cc2ccn([C@H](C)c3ccc4ccccc4c3)c12)c1ccc(C(=O)O)cc1 ; C[C@H](NC(=O)c1cc(-c2cc(F)ccc2F)cc2ccn([C@@H](C)c3ccc4ccccc4c3)c12)c1ccc(C(=O)O)cc1 ; C[C@H](NC(=O)c1cc(-c2cc(F)ccc2F)cc2ccn(C3(c4ccc5ccccc5c4)CC3)c12)c1ccc(C(=O)O)cc1 ; C[C@H](NC(=O)c1cc(-c2ccon2)cc2ccn(Cc3ccc4ccccc4c3)c12)c1ccc(C(=O)O)cc1]

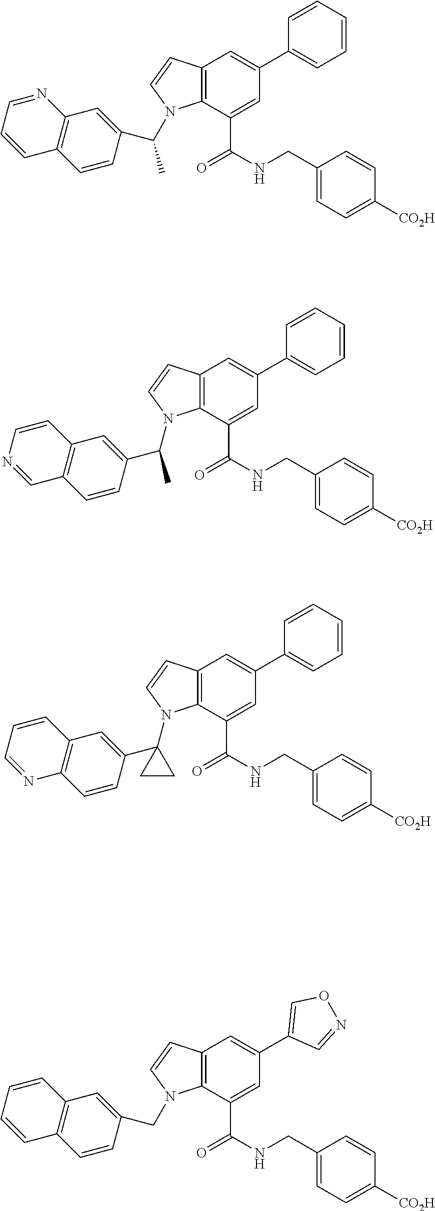 [SMILES: C[C@H](c1ccc2cccnc2c1)n1ccc2cc(-c3ccccc3)cc(C(=O)NCc3ccc(C(=O)O)cc3)c21 ; C[C@@H](c1ccc2cnccc2c1)n1ccc2cc(-c3ccccc3)cc(C(=O)NCc3ccc(C(=O)O)cc3)c21 ; O=C(O)c1ccc(CNC(=O)c2cc(-c3ccccc3)cc3ccn(C4(c5ccc6ncccc6c5)CC4)c23)cc1 ; O=C(O)c1ccc(CNC(=O)c2cc(-c3cnoc3)cc3ccn(Cc4ccc5ccccc5c4)c23)cc1]

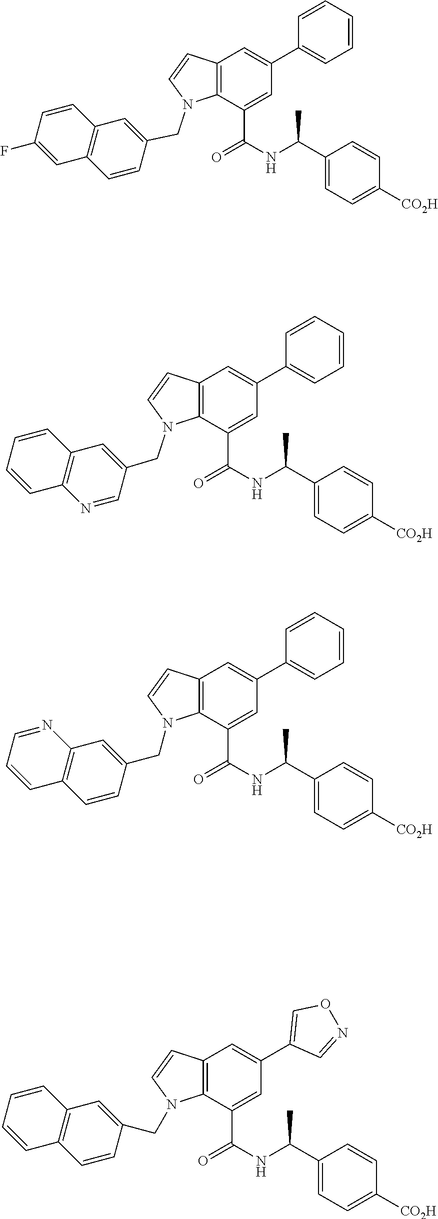 [SMILES: C[C@H](NC(=O)c1cc(-c2ccccc2)cc2ccn(Cc3ccc4cc(F)ccc4c3)c12)c1ccc(C(=O)O)cc1 ; C[C@H](NC(=O)c1cc(-c2ccccc2)cc2ccn(Cc3cnc4ccccc4c3)c12)c1ccc(C(=O)O)cc1 ; C[C@H](NC(=O)c1cc(-c2ccccc2)cc2ccn(Cc3ccc4cccnc4c3)c12)c1ccc(C(=O)O)cc1 ; C[C@H](NC(=O)c1cc(-c2cnoc2)cc2ccn(Cc3ccc4ccccc4c3)c12)c1ccc(C(=O)O)cc1]

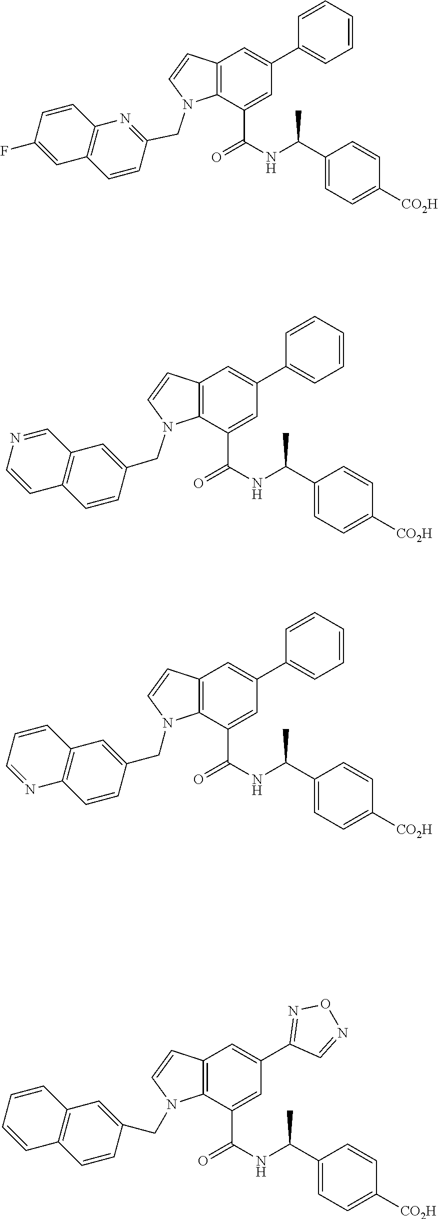 [SMILES: C[C@H](NC(=O)c1cc(-c2ccccc2)cc2ccn(Cc3ccc4cc(F)ccc4n3)c12)c1ccc(C(=O)O)cc1 ; C[C@H](NC(=O)c1cc(-c2ccccc2)cc2ccn(Cc3ccc4ccncc4c3)c12)c1ccc(C(=O)O)cc1 ; C[C@H](NC(=O)c1cc(-c2ccccc2)cc2ccn(Cc3ccc4ncccc4c3)c12)c1ccc(C(=O)O)cc1 ; C[C@H](NC(=O)c1cc(-c2cnon2)cc2ccn(Cc3ccc4ccccc4c3)c12)c1ccc(C(=O)O)cc1]

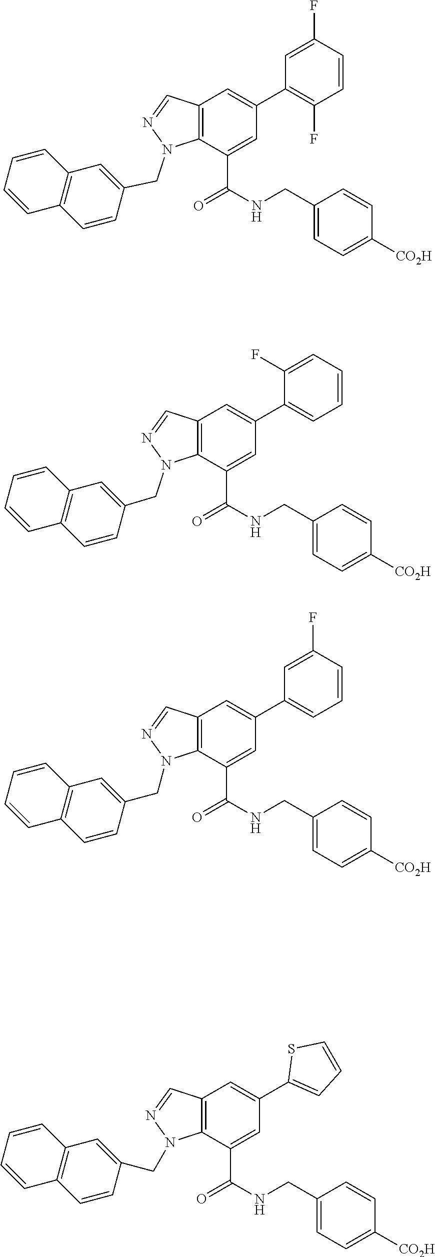 [SMILES: O=C(O)c1ccc(CNC(=O)c2cc(-c3cc(F)ccc3F)cc3cnn(Cc4ccc5ccccc5c4)c23)cc1 ; O=C(O)c1ccc(CNC(=O)c2cc(-c3ccccc3F)cc3cnn(Cc4ccc5ccccc5c4)c23)cc1 ; O=C(O)c1ccc(CNC(=O)c2cc(-c3cccc(F)c3)cc3cnn(Cc4ccc5ccccc5c4)c23)cc1 ; O=C(O)c1ccc(CNC(=O)c2cc(-c3cccs3)cc3cnn(Cc4ccc5ccccc5c4)c23)cc1]

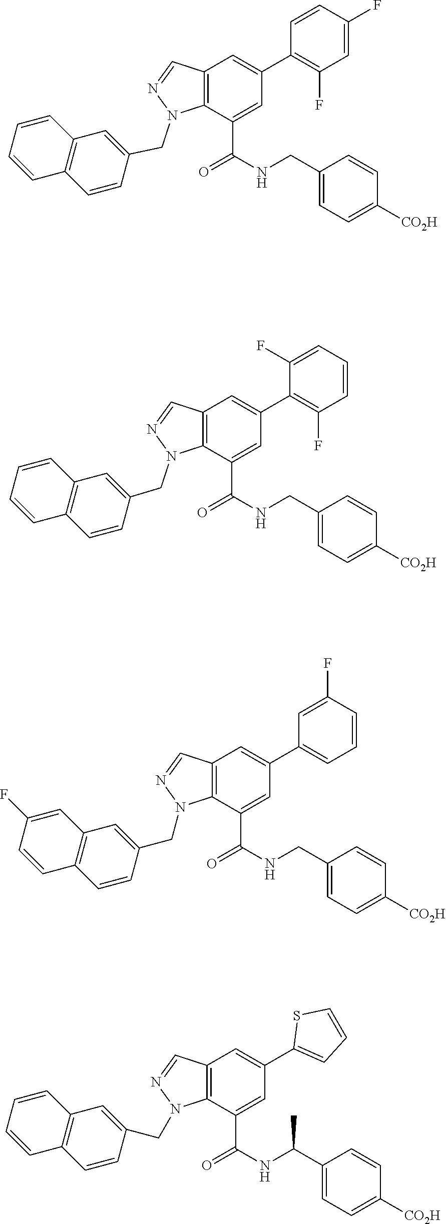 [SMILES: O=C(O)c1ccc(CNC(=O)c2cc(-c3ccc(F)cc3F)cc3cnn(Cc4ccc5ccccc5c4)c23)cc1 ; O=C(O)c1ccc(CNC(=O)c2cc(-c3c(F)cccc3F)cc3cnn(Cc4ccc5ccccc5c4)c23)cc1 ; O=C(O)c1ccc(CNC(=O)c2cc(-c3cccc(F)c3)cc3cnn(Cc4ccc5ccc(F)cc5c4)c23)cc1 ; C[C@H](NC(=O)c1cc(-c2cccs2)cc2cnn(Cc3ccc4ccccc4c3)c12)c1ccc(C(=O)O)cc1]

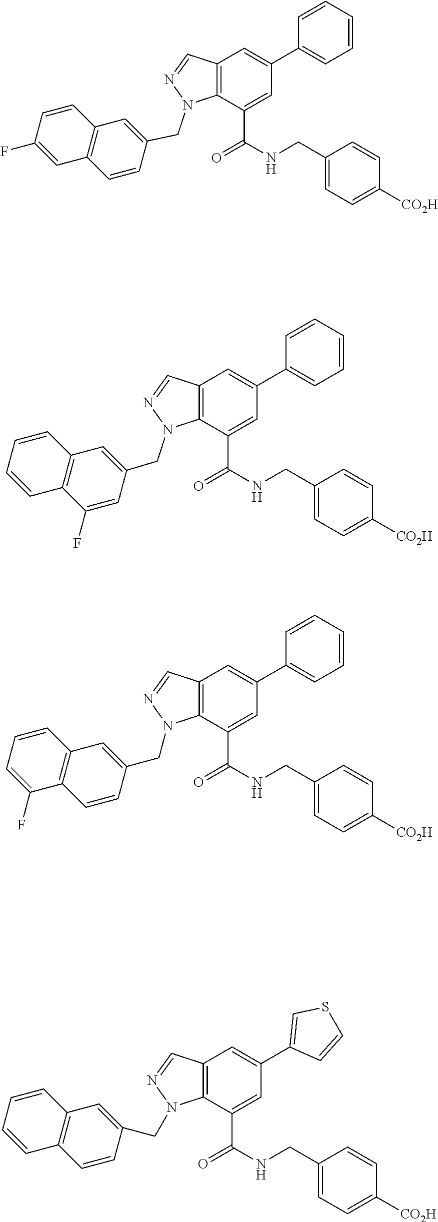 [SMILES: O=C(O)c1ccc(CNC(=O)c2cc(-c3ccccc3)cc3cnn(Cc4ccc5cc(F)ccc5c4)c23)cc1 ; O=C(O)c1ccc(CNC(=O)c2cc(-c3ccccc3)cc3cnn(Cc4cc(F)c5ccccc5c4)c23)cc1 ; O=C(O)c1ccc(CNC(=O)c2cc(-c3ccccc3)cc3cnn(Cc4ccc5c(F)cccc5c4)c23)cc1 ; O=C(O)c1ccc(CNC(=O)c2cc(-c3ccsc3)cc3cnn(Cc4ccc5ccccc5c4)c23)cc1]

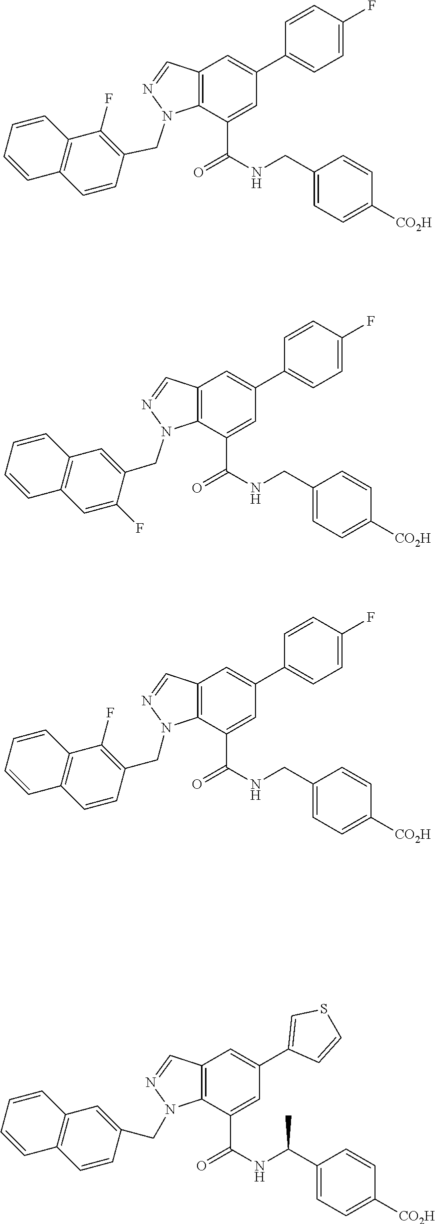 [SMILES: O=C(O)c1ccc(CNC(=O)c2cc(-c3ccc(F)cc3)cc3cnn(Cc4ccc5ccccc5c4F)c23)cc1 ; O=C(O)c1ccc(CNC(=O)c2cc(-c3ccc(F)cc3)cc3cnn(Cc4cc5ccccc5cc4F)c23)cc1 ; O=C(O)c1ccc(CNC(=O)c2cc(-c3ccc(F)cc3)cc3cnn(Cc4ccc5ccccc5c4F)c23)cc1 ; C[C@H](NC(=O)c1cc(-c2ccsc2)cc2cnn(Cc3ccc4ccccc4c3)c12)c1ccc(C(=O)O)cc1]

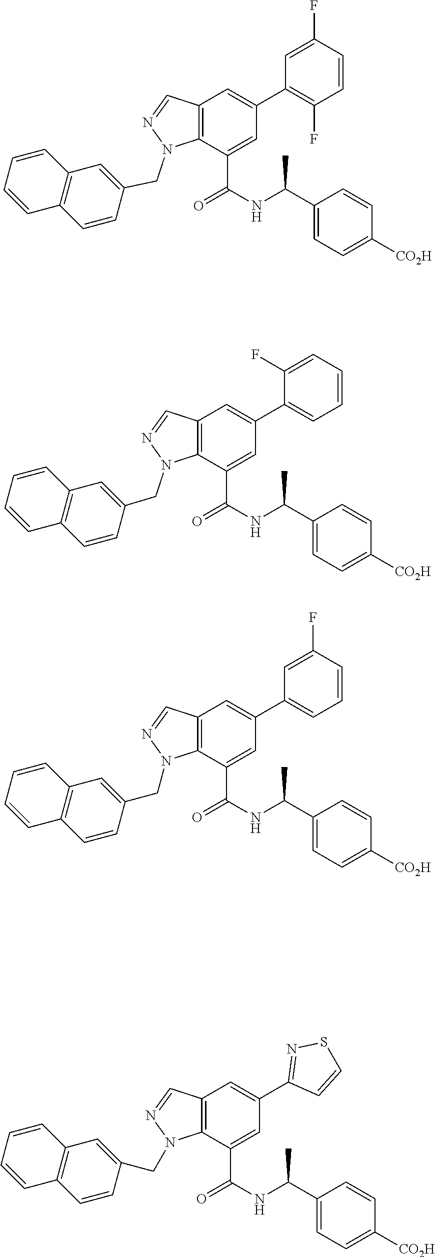 [SMILES: C[C@H](NC(=O)c1cc(-c2cc(F)ccc2F)cc2cnn(Cc3ccc4ccccc4c3)c12)c1ccc(C(=O)O)cc1 ; C[C@H](NC(=O)c1cc(-c2ccccc2F)cc2cnn(Cc3ccc4ccccc4c3)c12)c1ccc(C(=O)O)cc1 ; C[C@H](NC(=O)c1cc(-c2cccc(F)c2)cc2cnn(Cc3ccc4ccccc4c3)c12)c1ccc(C(=O)O)cc1 ; C[C@H](NC(=O)c1cc(-c2ccsn2)cc2cnn(Cc3ccc4ccccc4c3)c12)c1ccc(C(=O)O)cc1]

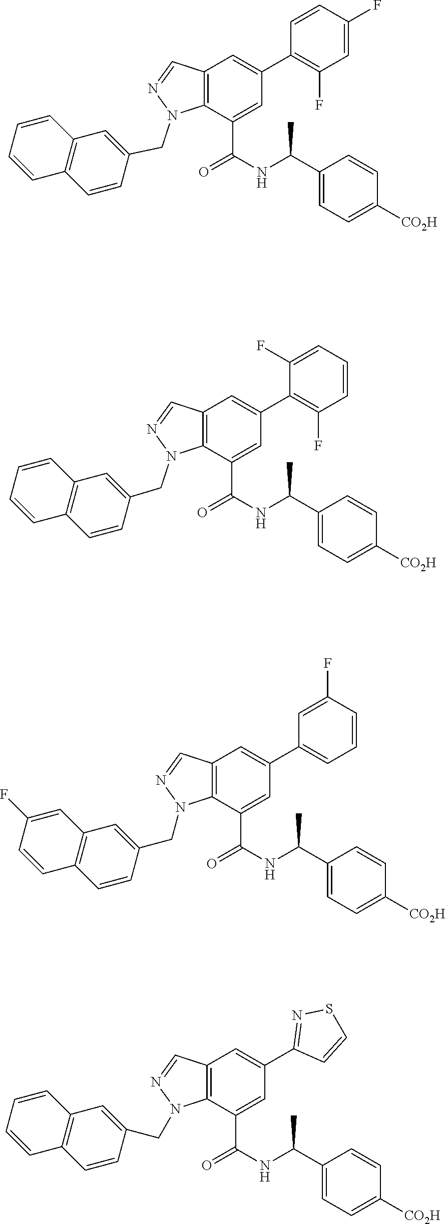 [SMILES: C[C@H](NC(=O)c1cc(-c2ccc(F)cc2F)cc2cnn(Cc3ccc4ccccc4c3)c12)c1ccc(C(=O)O)cc1 ; C[C@H](NC(=O)c1cc(-c2c(F)cccc2F)cc2cnn(Cc3ccc4ccccc4c3)c12)c1ccc(C(=O)O)cc1 ; C[C@H](NC(=O)c1cc(-c2cccc(F)c2)cc2cnn(Cc3ccc4ccc(F)cc4c3)c12)c1ccc(C(=O)O)cc1 ; C[C@H](NC(=O)c1cc(-c2ccsn2)cc2cnn(Cc3ccc4ccccc4c3)c12)c1ccc(C(=O)O)cc1]

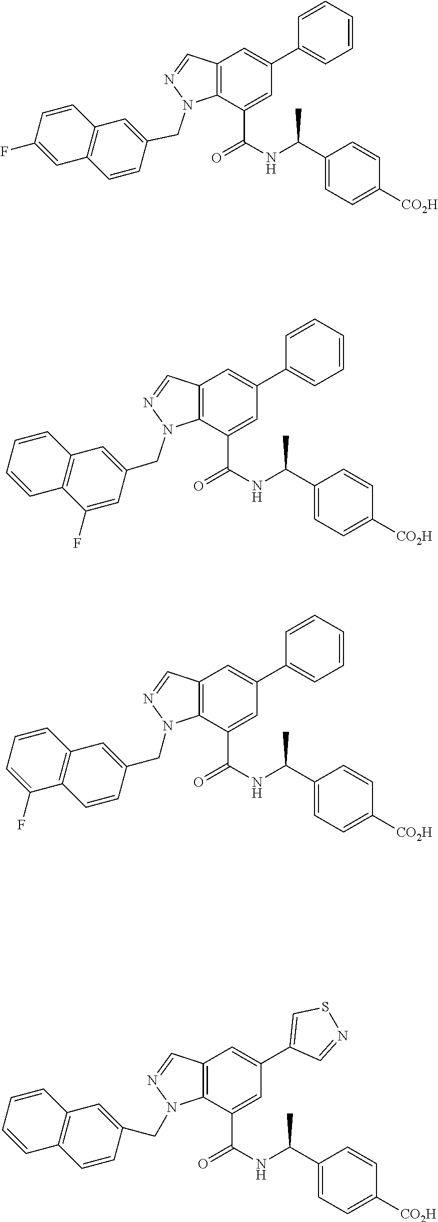 [SMILES: C[C@H](NC(=O)c1cc(-c2ccccc2)cc2cnn(Cc3ccc4cc(F)ccc4c3)c12)c1ccc(C(=O)O)cc1 ; C[C@H](NC(=O)c1cc(-c2ccccc2)cc2cnn(Cc3cc(F)c4ccccc4c3)c12)c1ccc(C(=O)O)cc1 ; C[C@H](NC(=O)c1cc(-c2ccccc2)cc2cnn(Cc3ccc4c(F)cccc4c3)c12)c1ccc(C(=O)O)cc1 ; C[C@H](NC(=O)c1cc(-c2cnsc2)cc2cnn(Cc3ccc4ccccc4c3)c12)c1ccc(C(=O)O)cc1]

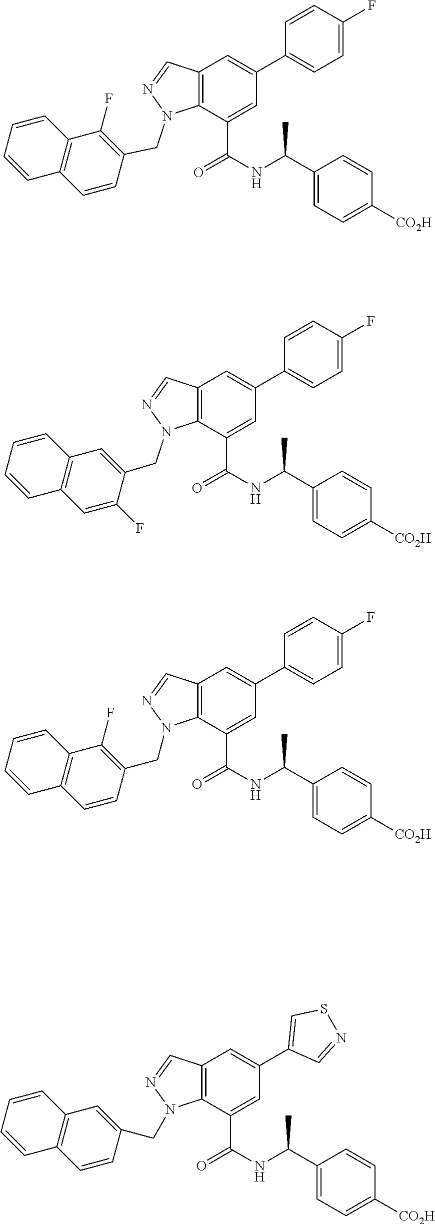 [SMILES: C[C@H](NC(=O)c1cc(-c2ccc(F)cc2)cc2cnn(Cc3ccc4ccccc4c3F)c12)c1ccc(C(=O)O)cc1 ; C[C@H](NC(=O)c1cc(-c2ccc(F)cc2)cc2cnn(Cc3cc4ccccc4cc3F)c12)c1ccc(C(=O)O)cc1 ; C[C@H](NC(=O)c1cc(-c2ccc(F)cc2)cc2cnn(Cc3ccc4ccccc4c3F)c12)c1ccc(C(=O)O)cc1 ; C[C@H](NC(=O)c1cc(-c2cnsc2)cc2cnn(Cc3ccc4ccccc4c3)c12)c1ccc(C(=O)O)cc1]

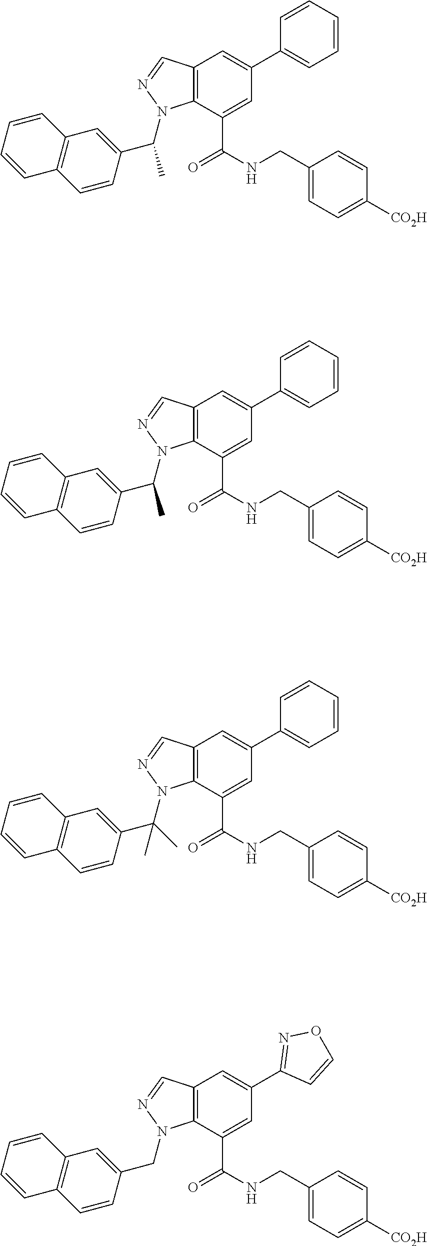 [SMILES: C[C@H](c1ccc2ccccc2c1)n1ncc2cc(-c3ccccc3)cc(C(=O)NCc3ccc(C(=O)O)cc3)c21 ; C[C@@H](c1ccc2ccccc2c1)n1ncc2cc(-c3ccccc3)cc(C(=O)NCc3ccc(C(=O)O)cc3)c21 ; CC(C)(c1ccc2ccccc2c1)n1ncc2cc(-c3ccccc3)cc(C(=O)NCc3ccc(C(=O)O)cc3)c21 ; O=C(O)c1ccc(CNC(=O)c2cc(-c3ccon3)cc3cnn(Cc4ccc5ccccc5c4)c23)cc1]

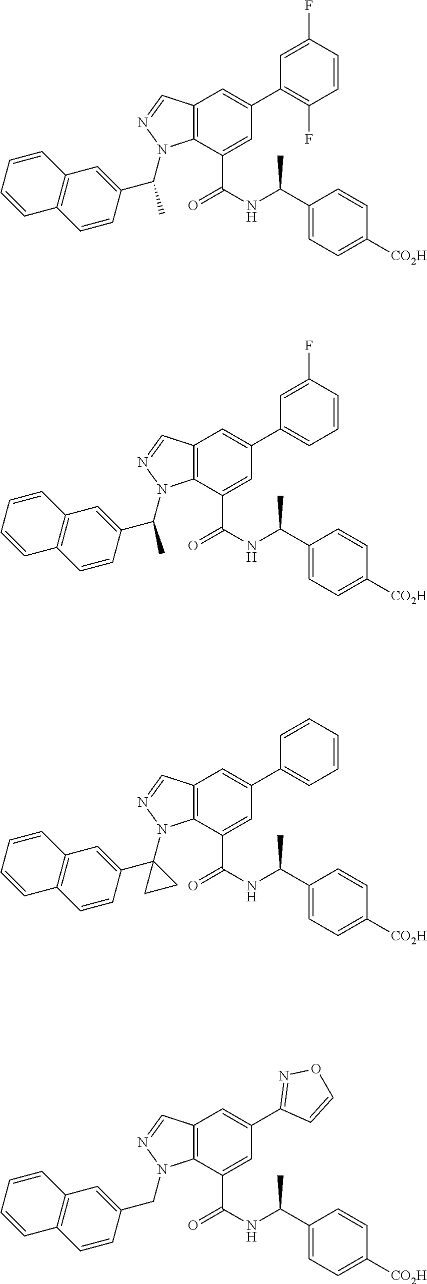 [SMILES: C[C@H](NC(=O)c1cc(-c2cc(F)ccc2F)cc2cnn([C@H](C)c3ccc4ccccc4c3)c12)c1ccc(C(=O)O)cc1 ; C[C@H](NC(=O)c1cc(-c2cccc(F)c2)cc2cnn([C@@H](C)c3ccc4ccccc4c3)c12)c1ccc(C(=O)O)cc1 ; C[C@H](NC(=O)c1cc(-c2ccccc2)cc2cnn(C3(c4ccc5ccccc5c4)CC3)c12)c1ccc(C(=O)O)cc1 ; C[C@H](NC(=O)c1cc(-c2ccon2)cc2cnn(Cc3ccc4ccccc4c3)c12)c1ccc(C(=O)O)cc1]

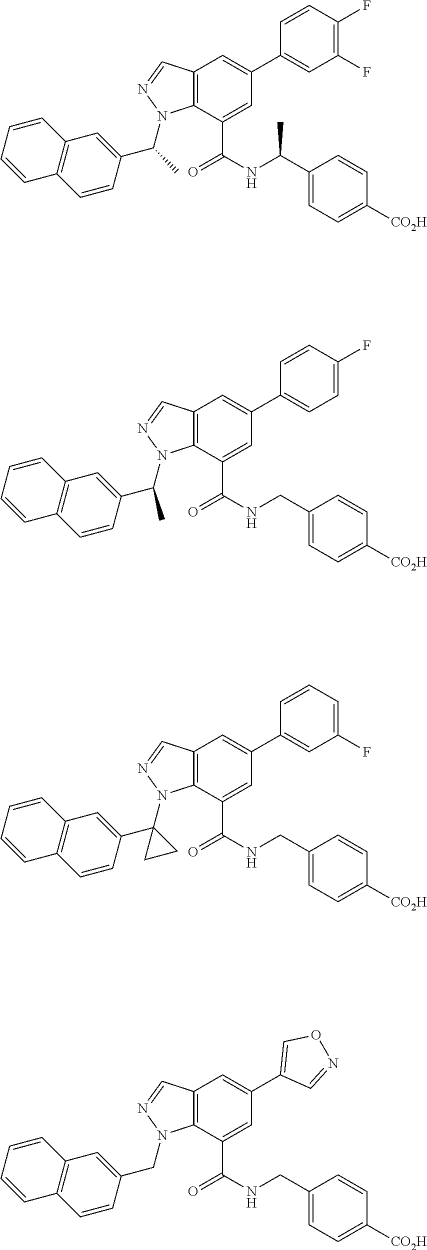 [SMILES: C[C@H](NC(=O)c1cc(-c2ccc(F)c(F)c2)cc2cnn([C@H](C)c3ccc4ccccc4c3)c12)c1ccc(C(=O)O)cc1 ; C[C@@H](c1ccc2ccccc2c1)n1ncc2cc(-c3ccc(F)cc3)cc(C(=O)NCc3ccc(C(=O)O)cc3)c21 ; O=C(O)c1ccc(CNC(=O)c2cc(-c3cccc(F)c3)cc3cnn(C4(c5ccc6ccccc6c5)CC4)c23)cc1 ; O=C(O)c1ccc(CNC(=O)c2cc(-c3cnoc3)cc3cnn(Cc4ccc5ccccc5c4)c23)cc1]

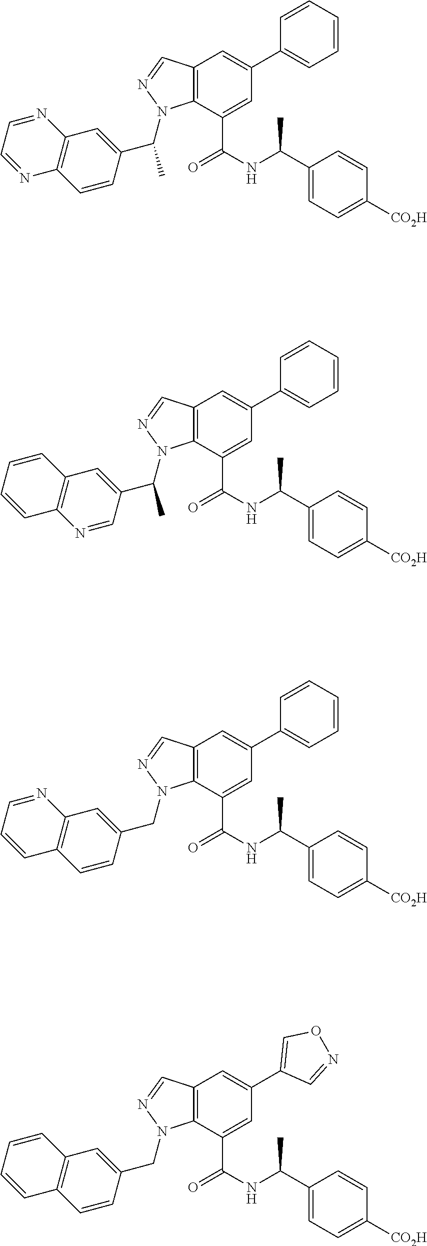 [SMILES: C[C@H](NC(=O)c1cc(-c2ccccc2)cc2cnn([C@H](C)c3ccc4nccnc4c3)c12)c1ccc(C(=O)O)cc1 ; C[C@H](NC(=O)c1cc(-c2ccccc2)cc2cnn([C@@H](C)c3cnc4ccccc4c3)c12)c1ccc(C(=O)O)cc1 ; C[C@H](NC(=O)c1cc(-c2ccccc2)cc2cnn(Cc3ccc4cccnc4c3)c12)c1ccc(C(=O)O)cc1 ; C[C@H](NC(=O)c1cc(-c2cnoc2)cc2cnn(Cc3ccc4ccccc4c3)c12)c1ccc(C(=O)O)cc1]

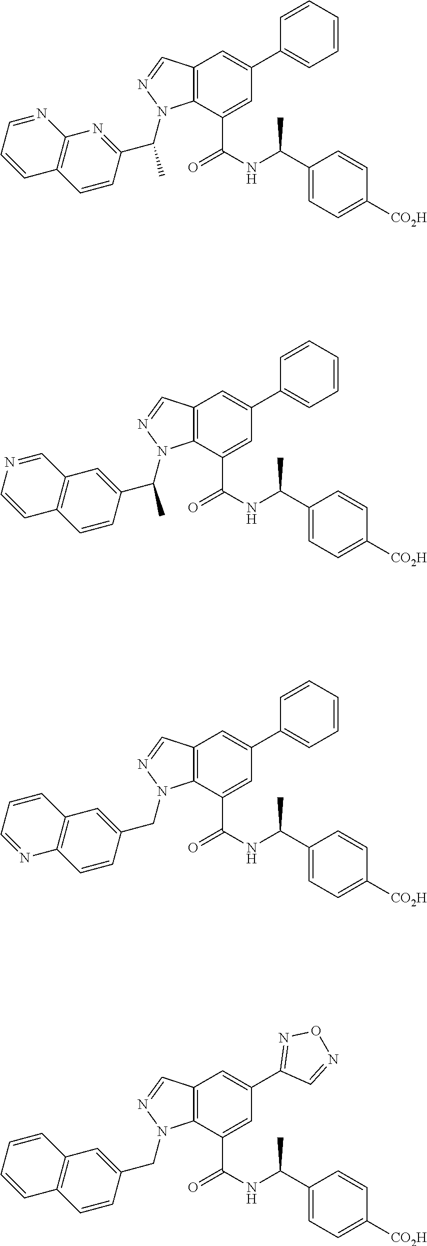 [SMILES: C[C@H](NC(=O)c1cc(-c2ccccc2)cc2cnn([C@H](C)c3ccc4cccnc4n3)c12)c1ccc(C(=O)O)cc1 ; C[C@H](NC(=O)c1cc(-c2ccccc2)cc2cnn([C@@H](C)c3ccc4ccncc4c3)c12)c1ccc(C(=O)O)cc1 ; C[C@H](NC(=O)c1cc(-c2ccccc2)cc2cnn(Cc3ccc4ncccc4c3)c12)c1ccc(C(=O)O)cc1 ; C[C@H](NC(=O)c1cc(-c2cnon2)cc2cnn(Cc3ccc4ccccc4c3)c12)c1ccc(C(=O)O)cc1]

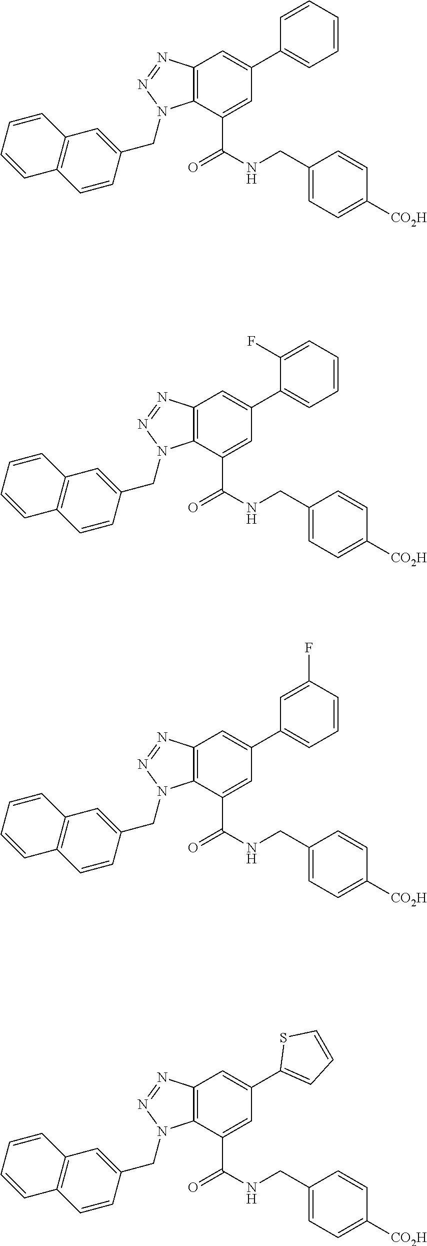 [SMILES: O=C(O)c1ccc(CNC(=O)c2cc(-c3ccccc3)cc3nnn(Cc4ccc5ccccc5c4)c23)cc1 ; O=C(O)c1ccc(CNC(=O)c2cc(-c3ccccc3F)cc3nnn(Cc4ccc5ccccc5c4)c23)cc1 ; O=C(O)c1ccc(CNC(=O)c2cc(-c3cccc(F)c3)cc3nnn(Cc4ccc5ccccc5c4)c23)cc1 ; O=C(O)c1ccc(CNC(=O)c2cc(-c3cccs3)cc3nnn(Cc4ccc5ccccc5c4)c23)cc1]

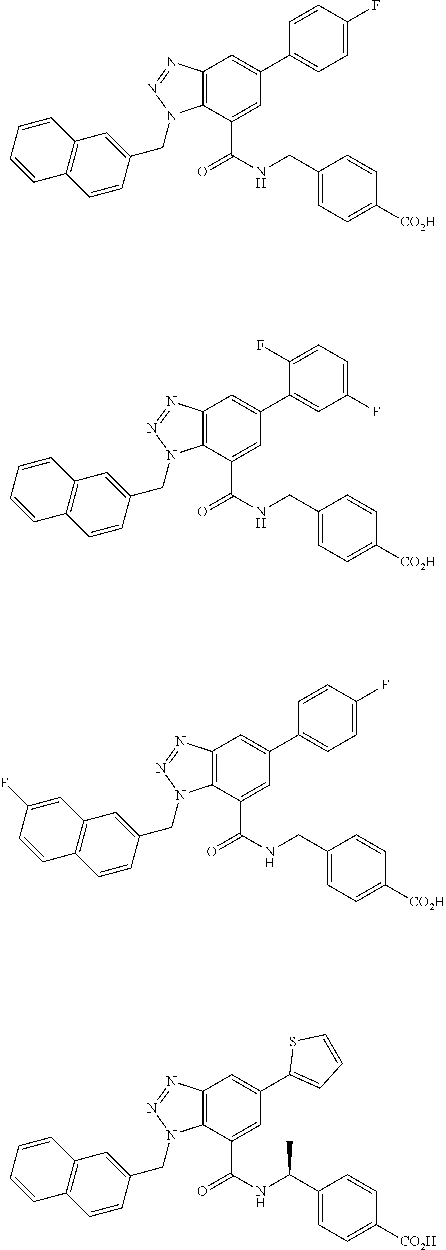 [SMILES: O=C(O)c1ccc(CNC(=O)c2cc(-c3ccc(F)cc3)cc3nnn(Cc4ccc5ccccc5c4)c23)cc1 ; O=C(O)c1ccc(CNC(=O)c2cc(-c3cc(F)ccc3F)cc3nnn(Cc4ccc5ccccc5c4)c23)cc1 ; O=C(O)c1ccc(CNC(=O)c2cc(-c3ccc(F)cc3)cc3nnn(Cc4ccc5ccc(F)cc5c4)c23)cc1 ; C[C@H](NC(=O)c1cc(-c2cccs2)cc2nnn(Cc3ccc4ccccc4c3)c12)c1ccc(C(=O)O)cc1]

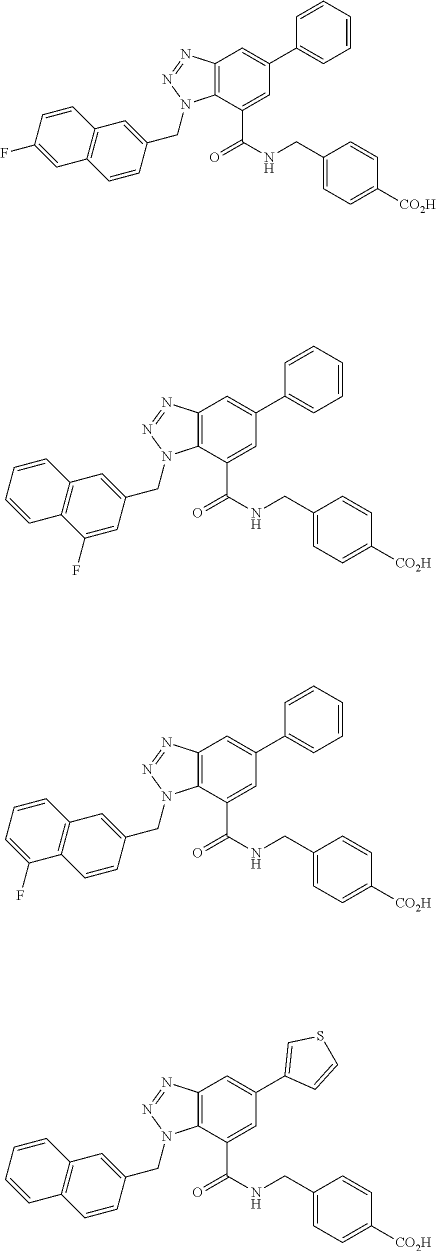 [SMILES: O=C(O)c1ccc(CNC(=O)c2cc(-c3ccccc3)cc3nnn(Cc4ccc5cc(F)ccc5c4)c23)cc1 ; O=C(O)c1ccc(CNC(=O)c2cc(-c3ccccc3)cc3nnn(Cc4cc(F)c5ccccc5c4)c23)cc1 ; O=C(O)c1ccc(CNC(=O)c2cc(-c3ccccc3)cc3nnn(Cc4ccc5c(F)cccc5c4)c23)cc1 ; O=C(O)c1ccc(CNC(=O)c2cc(-c3ccsc3)cc3nnn(Cc4ccc5ccccc5c4)c23)cc1]

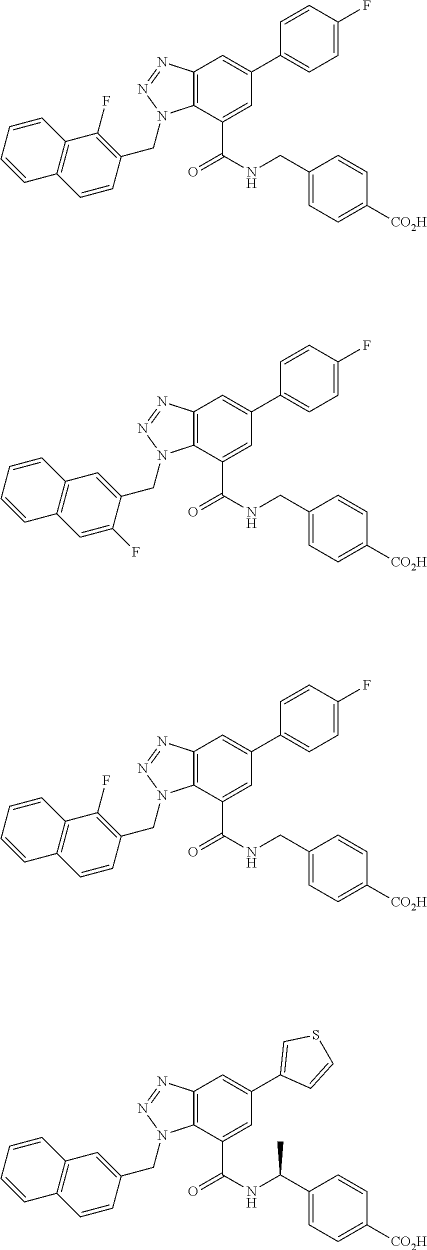 [SMILES: O=C(O)c1ccc(CNC(=O)c2cc(-c3ccc(F)cc3)cc3nnn(Cc4ccc5ccccc5c4F)c23)cc1 ; O=C(O)c1ccc(CNC(=O)c2cc(-c3ccc(F)cc3)cc3nnn(Cc4cc5ccccc5cc4F)c23)cc1 ; O=C(O)c1ccc(CNC(=O)c2cc(-c3ccc(F)cc3)cc3nnn(Cc4ccc5ccccc5c4F)c23)cc1 ; C[C@H](NC(=O)c1cc(-c2ccsc2)cc2nnn(Cc3ccc4ccccc4c3)c12)c1ccc(C(=O)O)cc1]

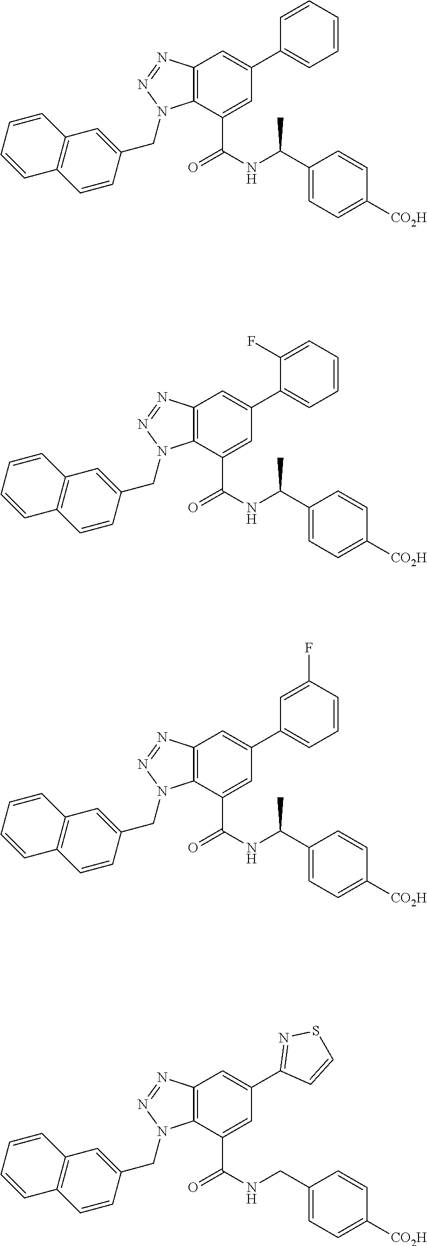 [SMILES: C[C@H](NC(=O)c1cc(-c2ccccc2)cc2nnn(Cc3ccc4ccccc4c3)c12)c1ccc(C(=O)O)cc1 ; C[C@H](NC(=O)c1cc(-c2ccccc2F)cc2nnn(Cc3ccc4ccccc4c3)c12)c1ccc(C(=O)O)cc1 ; C[C@H](NC(=O)c1cc(-c2cccc(F)c2)cc2nnn(Cc3ccc4ccccc4c3)c12)c1ccc(C(=O)O)cc1 ; O=C(O)c1ccc(CNC(=O)c2cc(-c3ccsn3)cc3nnn(Cc4ccc5ccccc5c4)c23)cc1]

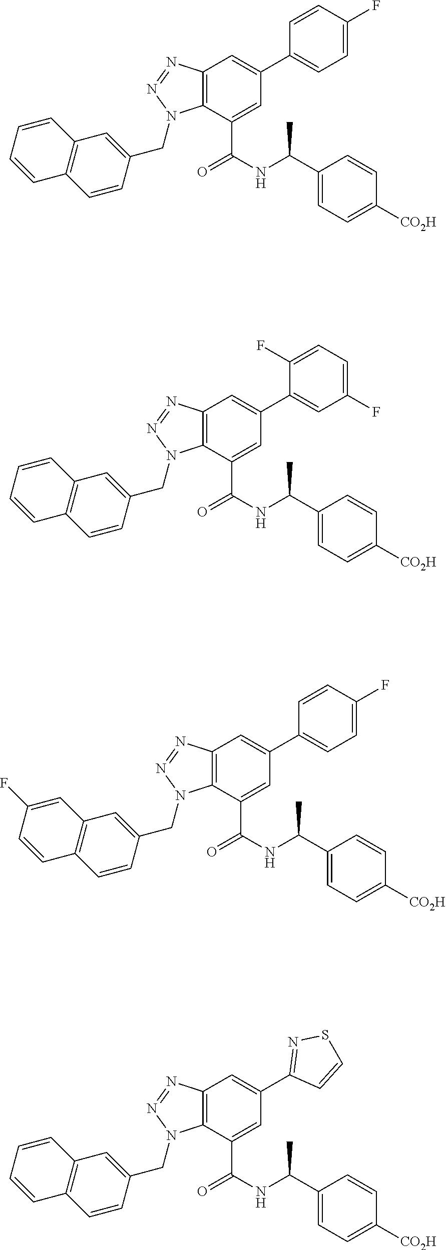 [SMILES: C[C@H](NC(=O)c1cc(-c2ccc(F)cc2)cc2nnn(Cc3ccc4ccccc4c3)c12)c1ccc(C(=O)O)cc1 ; C[C@H](NC(=O)c1cc(-c2cc(F)ccc2F)cc2nnn(Cc3ccc4ccccc4c3)c12)c1ccc(C(=O)O)cc1 ; C[C@H](NC(=O)c1cc(-c2ccc(F)cc2)cc2nnn(Cc3ccc4ccc(F)cc4c3)c12)c1ccc(C(=O)O)cc1 ; C[C@H](NC(=O)c1cc(-c2ccsn2)cc2nnn(Cc3ccc4ccccc4c3)c12)c1ccc(C(=O)O)cc1]

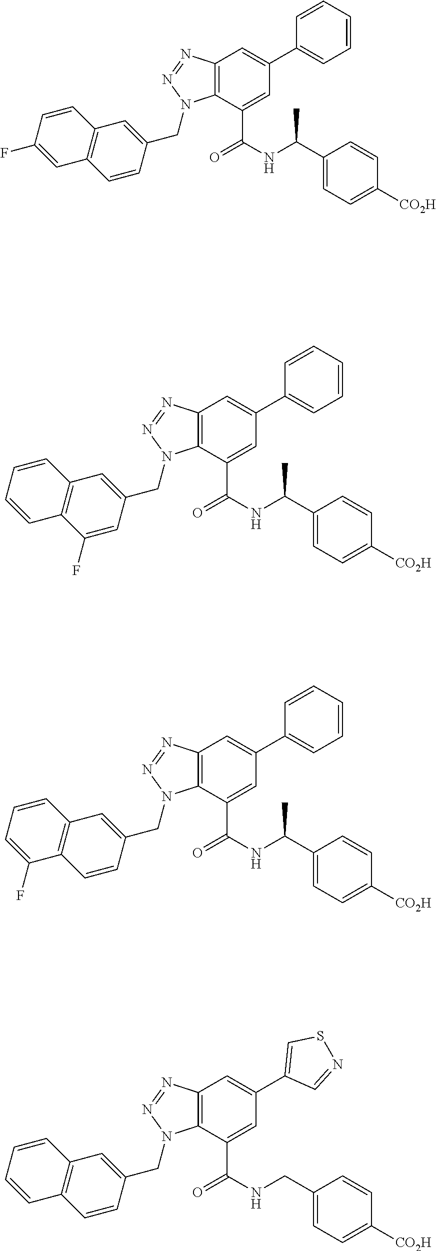 [SMILES: C[C@H](NC(=O)c1cc(-c2ccccc2)cc2nnn(Cc3ccc4cc(F)ccc4c3)c12)c1ccc(C(=O)O)cc1 ; C[C@H](NC(=O)c1cc(-c2ccccc2)cc2nnn(Cc3cc(F)c4ccccc4c3)c12)c1ccc(C(=O)O)cc1 ; C[C@H](NC(=O)c1cc(-c2ccccc2)cc2nnn(Cc3ccc4c(F)cccc4c3)c12)c1ccc(C(=O)O)cc1 ; O=C(O)c1ccc(CNC(=O)c2cc(-c3cnsc3)cc3nnn(Cc4ccc5ccccc5c4)c23)cc1]

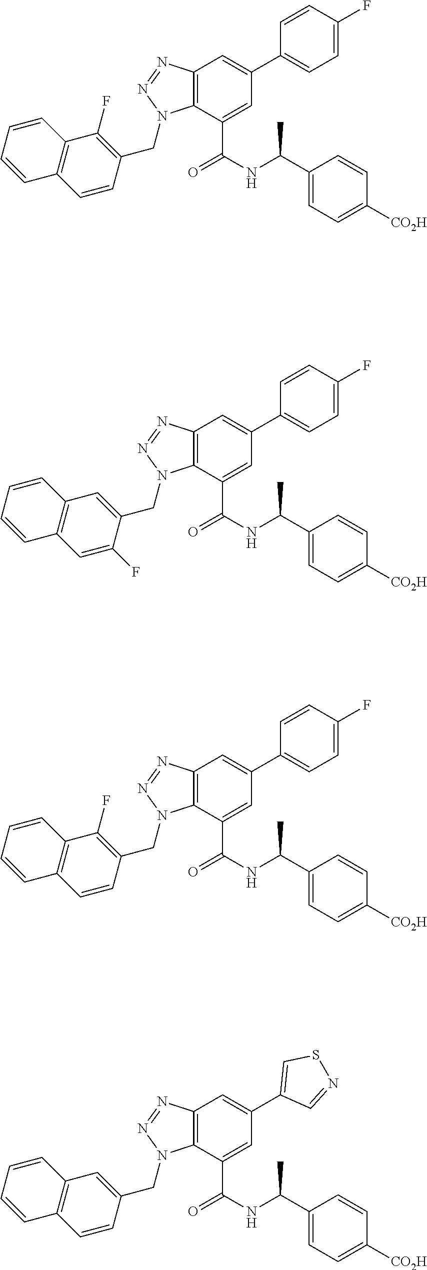 [SMILES: C[C@H](NC(=O)c1cc(-c2ccc(F)cc2)cc2nnn(Cc3ccc4ccccc4c3F)c12)c1ccc(C(=O)O)cc1 ; C[C@H](NC(=O)c1cc(-c2ccc(F)cc2)cc2nnn(Cc3cc4ccccc4cc3F)c12)c1ccc(C(=O)O)cc1 ; C[C@H](NC(=O)c1cc(-c2ccc(F)cc2)cc2nnn(Cc3ccc4ccccc4c3F)c12)c1ccc(C(=O)O)cc1 ; C[C@H](NC(=O)c1cc(-c2cnsc2)cc2nnn(Cc3ccc4ccccc4c3)c12)c1ccc(C(=O)O)cc1]

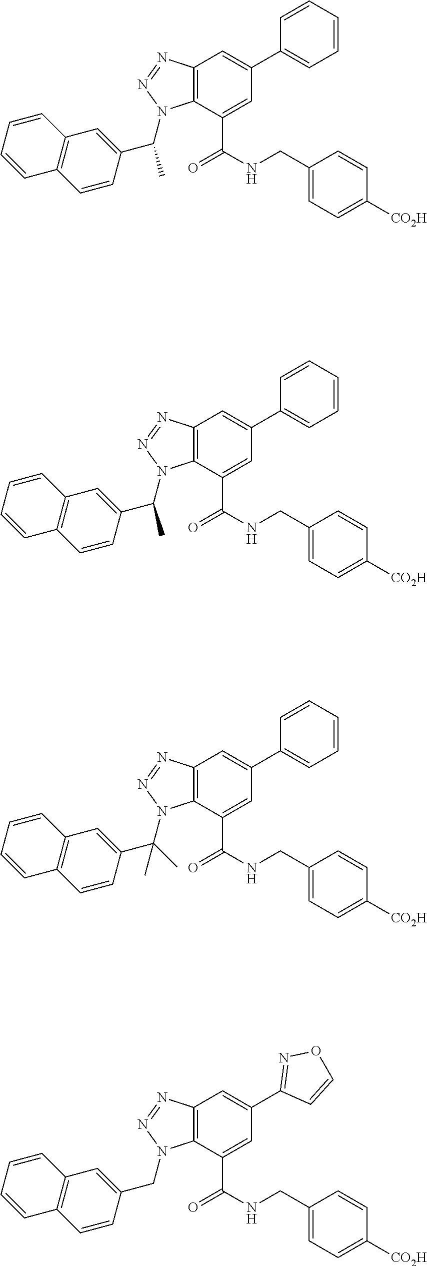 [SMILES: C[C@H](c1ccc2ccccc2c1)n1nnc2cc(-c3ccccc3)cc(C(=O)NCc3ccc(C(=O)O)cc3)c21 ; C[C@@H](c1ccc2ccccc2c1)n1nnc2cc(-c3ccccc3)cc(C(=O)NCc3ccc(C(=O)O)cc3)c21 ; CC(C)(c1ccc2ccccc2c1)n1nnc2cc(-c3ccccc3)cc(C(=O)NCc3ccc(C(=O)O)cc3)c21 ; O=C(O)c1ccc(CNC(=O)c2cc(-c3ccon3)cc3nnn(Cc4ccc5ccccc5c4)c23)cc1]

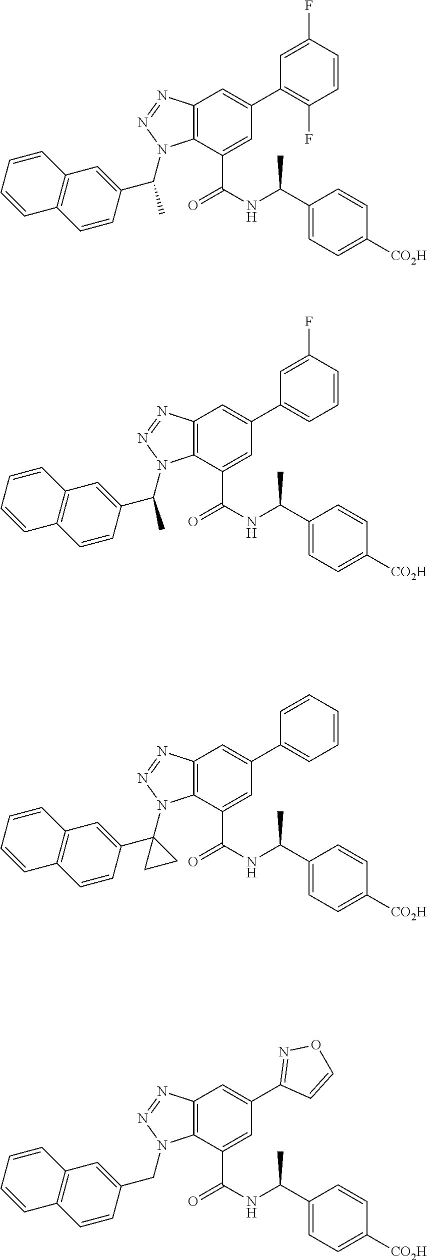 [SMILES: C[C@H](NC(=O)c1cc(-c2cc(F)ccc2F)cc2nnn([C@H](C)c3ccc4ccccc4c3)c12)c1ccc(C(=O)O)cc1 ; C[C@H](NC(=O)c1cc(-c2cccc(F)c2)cc2nnn([C@@H](C)c3ccc4ccccc4c3)c12)c1ccc(C(=O)O)cc1 ; C[C@H](NC(=O)c1cc(-c2ccccc2)cc2nnn(C3(c4ccc5ccccc5c4)CC3)c12)c1ccc(C(=O)O)cc1 ; C[C@H](NC(=O)c1cc(-c2ccon2)cc2nnn(Cc3ccc4ccccc4c3)c12)c1ccc(C(=O)O)cc1]

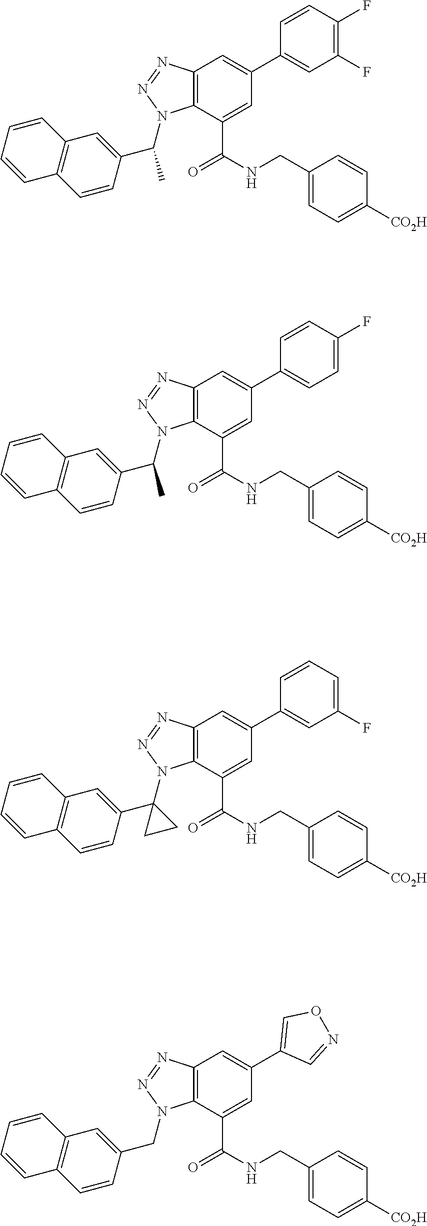 [SMILES: C[C@H](c1ccc2ccccc2c1)n1nnc2cc(-c3ccc(F)c(F)c3)cc(C(=O)NCc3ccc(C(=O)O)cc3)c21 ; C[C@@H](c1ccc2ccccc2c1)n1nnc2cc(-c3ccc(F)cc3)cc(C(=O)NCc3ccc(C(=O)O)cc3)c21 ; O=C(O)c1ccc(CNC(=O)c2cc(-c3cccc(F)c3)cc3nnn(C4(c5ccc6ccccc6c5)CC4)c23)cc1 ; O=C(O)c1ccc(CNC(=O)c2cc(-c3cnoc3)cc3nnn(Cc4ccc5ccccc5c4)c23)cc1]

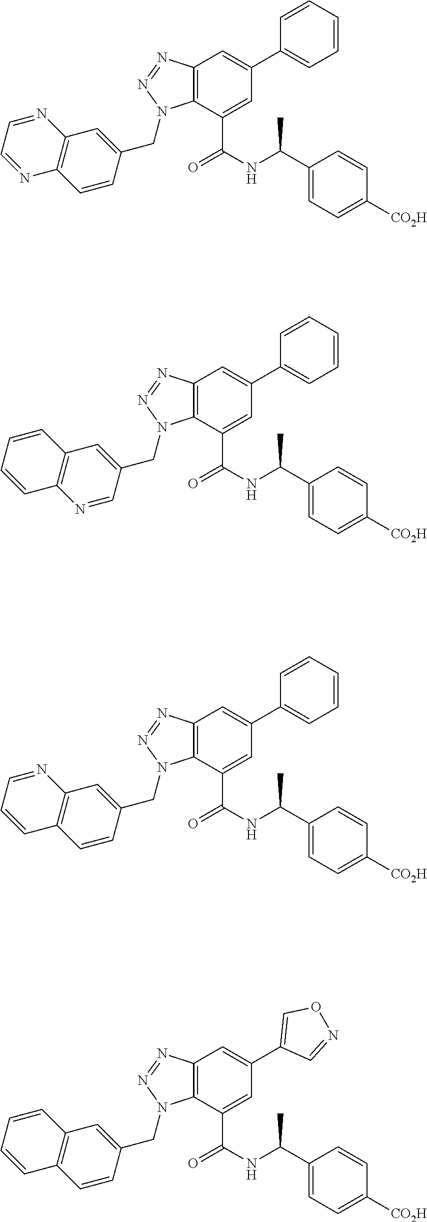 [SMILES: C[C@H](NC(=O)c1cc(-c2ccccc2)cc2nnn(Cc3ccc4nccnc4c3)c12)c1ccc(C(=O)O)cc1 ; C[C@H](NC(=O)c1cc(-c2ccccc2)cc2nnn(Cc3cnc4ccccc4c3)c12)c1ccc(C(=O)O)cc1 ; C[C@H](NC(=O)c1cc(-c2ccccc2)cc2nnn(Cc3ccc4cccnc4c3)c12)c1ccc(C(=O)O)cc1 ; C[C@H](NC(=O)c1cc(-c2cnoc2)cc2nnn(Cc3ccc4ccccc4c3)c12)c1ccc(C(=O)O)cc1]

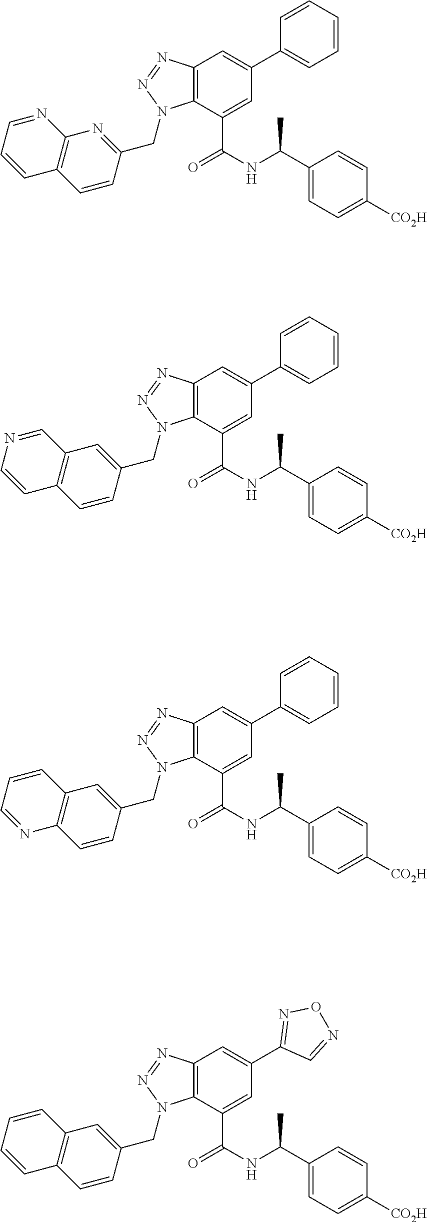 [SMILES: C[C@H](NC(=O)c1cc(-c2ccccc2)cc2nnn(Cc3ccc4cccnc4n3)c12)c1ccc(C(=O)O)cc1 ; C[C@H](NC(=O)c1cc(-c2ccccc2)cc2nnn(Cc3ccc4ccncc4c3)c12)c1ccc(C(=O)O)cc1 ; C[C@H](NC(=O)c1cc(-c2ccccc2)cc2nnn(Cc3ccc4ncccc4c3)c12)c1ccc(C(=O)O)cc1 ; C[C@H](NC(=O)c1cc(-c2cnon2)cc2nnn(Cc3ccc4ccccc4c3)c12)c1ccc(C(=O)O)cc1]

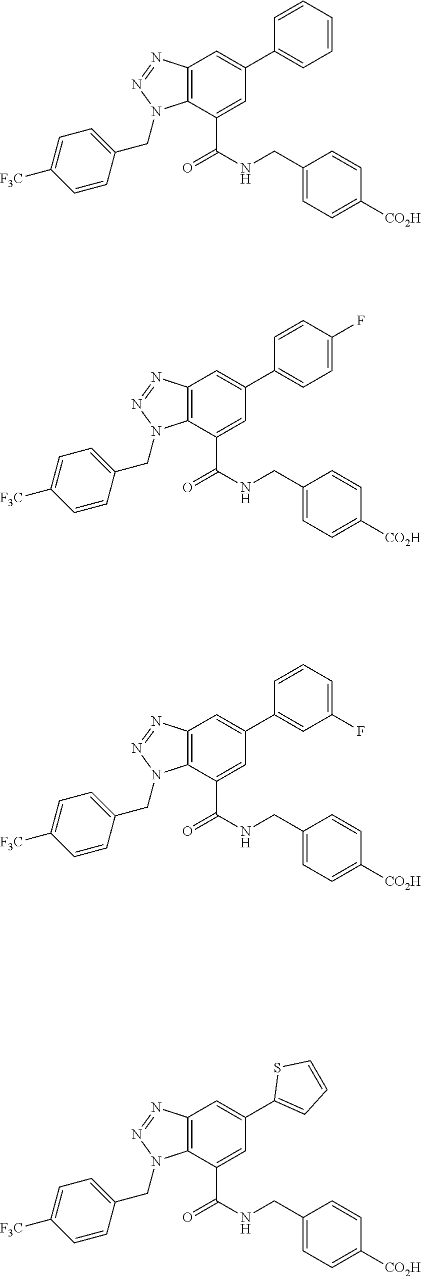 [SMILES: O=C(O)c1ccc(CNC(=O)c2cc(-c3ccccc3)cc3nnn(Cc4ccc(C(F)(F)F)cc4)c23)cc1 ; O=C(O)c1ccc(CNC(=O)c2cc(-c3ccc(F)cc3)cc3nnn(Cc4ccc(C(F)(F)F)cc4)c23)cc1 ; O=C(O)c1ccc(CNC(=O)c2cc(-c3cccc(F)c3)cc3nnn(Cc4ccc(C(F)(F)F)cc4)c23)cc1 ; O=C(O)c1ccc(CNC(=O)c2cc(-c3cccs3)cc3nnn(Cc4ccc(C(F)(F)F)cc4)c23)cc1]

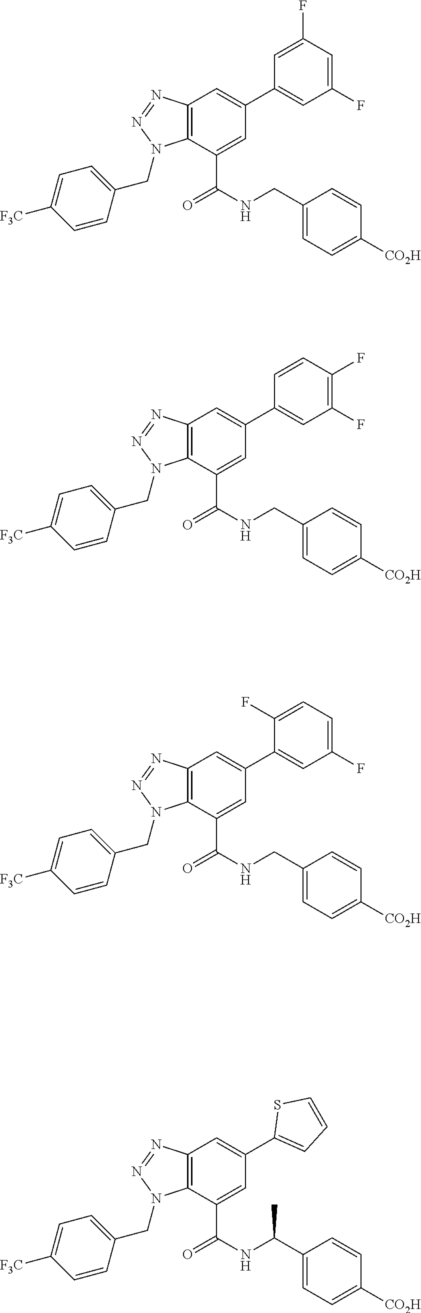 [SMILES: O=C(O)c1ccc(CNC(=O)c2cc(-c3cc(F)cc(F)c3)cc3nnn(Cc4ccc(C(F)(F)F)cc4)c23)cc1 ; O=C(O)c1ccc(CNC(=O)c2cc(-c3ccc(F)c(F)c3)cc3nnn(Cc4ccc(C(F)(F)F)cc4)c23)cc1 ; O=C(O)c1ccc(CNC(=O)c2cc(-c3cc(F)ccc3F)cc3nnn(Cc4ccc(C(F)(F)F)cc4)c23)cc1 ; C[C@H](NC(=O)c1cc(-c2cccs2)cc2nnn(Cc3ccc(C(F)(F)F)cc3)c12)c1ccc(C(=O)O)cc1]

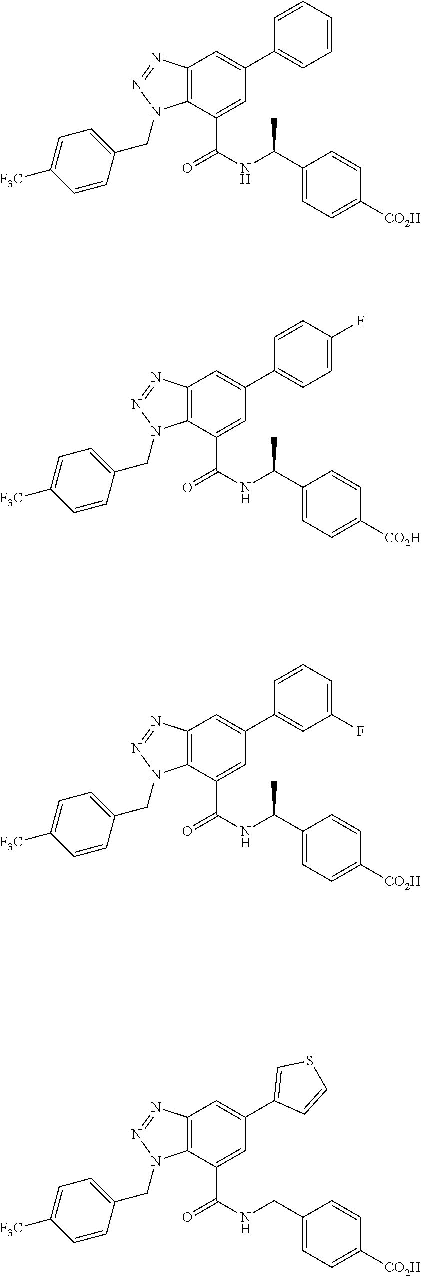 [SMILES: C[C@H](NC(=O)c1cc(-c2ccccc2)cc2nnn(Cc3ccc(C(F)(F)F)cc3)c12)c1ccc(C(=O)O)cc1 ; C[C@H](NC(=O)c1cc(-c2ccc(F)cc2)cc2nnn(Cc3ccc(C(F)(F)F)cc3)c12)c1ccc(C(=O)O)cc1 ; C[C@H](NC(=O)c1cc(-c2cccc(F)c2)cc2nnn(Cc3ccc(C(F)(F)F)cc3)c12)c1ccc(C(=O)O)cc1 ; O=C(O)c1ccc(CNC(=O)c2cc(-c3ccsc3)cc3nnn(Cc4ccc(C(F)(F)F)cc4)c23)cc1]

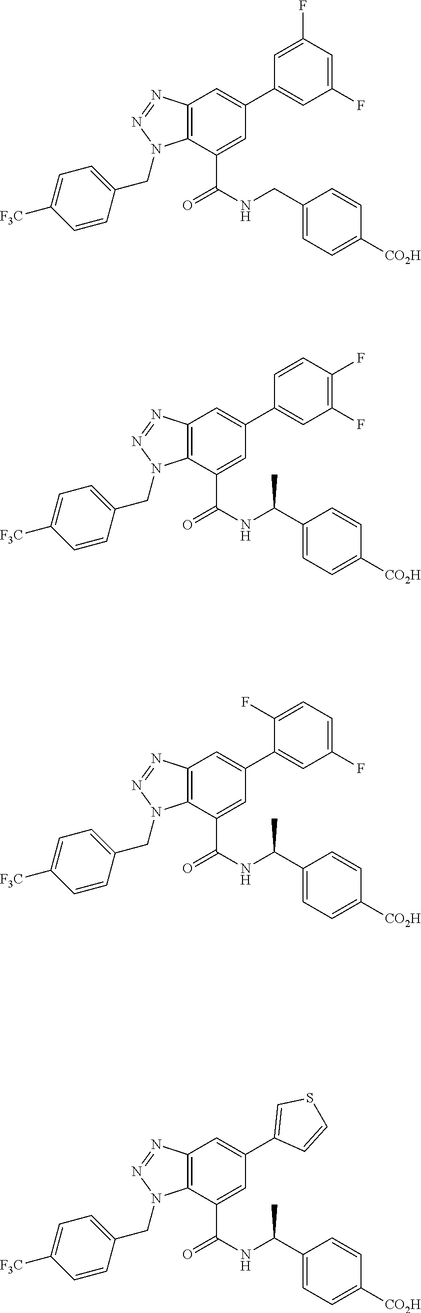 [SMILES: O=C(O)c1ccc(CNC(=O)c2cc(-c3cc(F)cc(F)c3)cc3nnn(Cc4ccc(C(F)(F)F)cc4)c23)cc1 ; C[C@H](NC(=O)c1cc(-c2ccc(F)c(F)c2)cc2nnn(Cc3ccc(C(F)(F)F)cc3)c12)c1ccc(C(=O)O)cc1 ; C[C@H](NC(=O)c1cc(-c2cc(F)ccc2F)cc2nnn(Cc3ccc(C(F)(F)F)cc3)c12)c1ccc(C(=O)O)cc1 ; C[C@H](NC(=O)c1cc(-c2ccsc2)cc2nnn(Cc3ccc(C(F)(F)F)cc3)c12)c1ccc(C(=O)O)cc1]

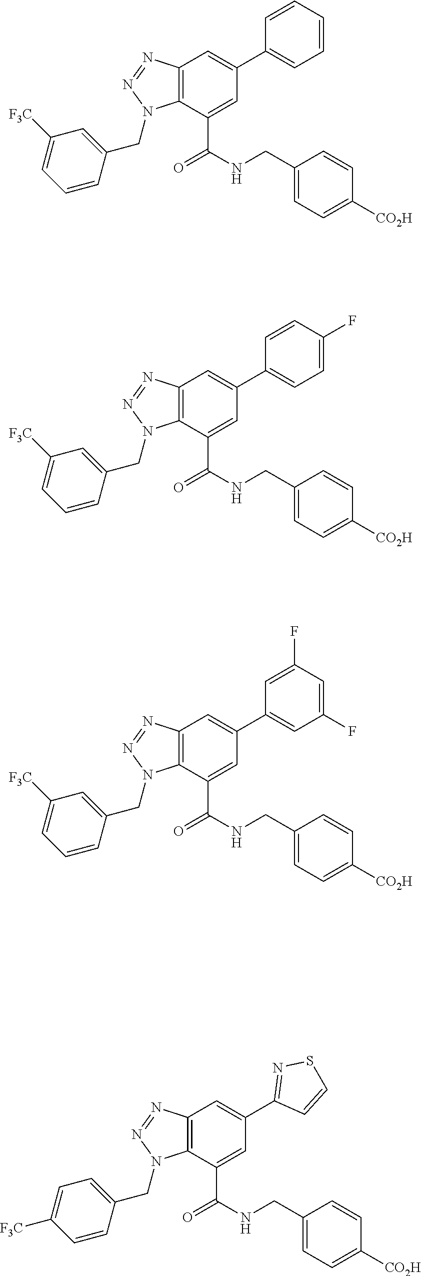 [SMILES: O=C(O)c1ccc(CNC(=O)c2cc(-c3ccccc3)cc3nnn(Cc4cccc(C(F)(F)F)c4)c23)cc1 ; O=C(O)c1ccc(CNC(=O)c2cc(-c3ccc(F)cc3)cc3nnn(Cc4cccc(C(F)(F)F)c4)c23)cc1 ; O=C(O)c1ccc(CNC(=O)c2cc(-c3cc(F)cc(F)c3)cc3nnn(Cc4cccc(C(F)(F)F)c4)c23)cc1 ; O=C(O)c1ccc(CNC(=O)c2cc(-c3ccsn3)cc3nnn(Cc4ccc(C(F)(F)F)cc4)c23)cc1]

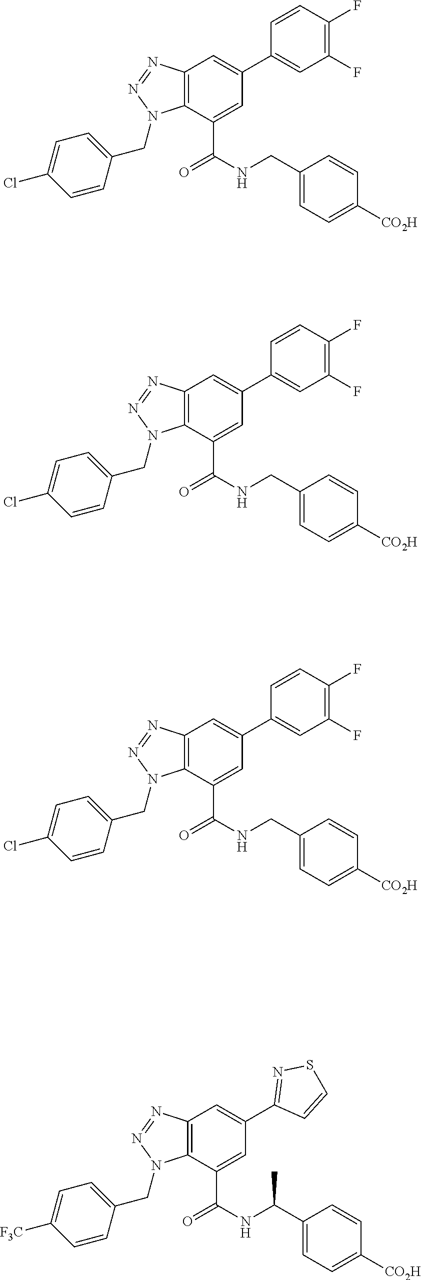 [SMILES: O=C(O)c1ccc(CNC(=O)c2cc(-c3ccc(F)c(F)c3)cc3nnn(Cc4ccc(Cl)cc4)c23)cc1 ; O=C(O)c1ccc(CNC(=O)c2cc(-c3ccc(F)c(F)c3)cc3nnn(Cc4ccc(Cl)cc4)c23)cc1 ; O=C(O)c1ccc(CNC(=O)c2cc(-c3ccc(F)c(F)c3)cc3nnn(Cc4ccc(Cl)cc4)c23)cc1 ; C[C@H](NC(=O)c1cc(-c2ccsn2)cc2nnn(Cc3ccc(C(F)(F)F)cc3)c12)c1ccc(C(=O)O)cc1]

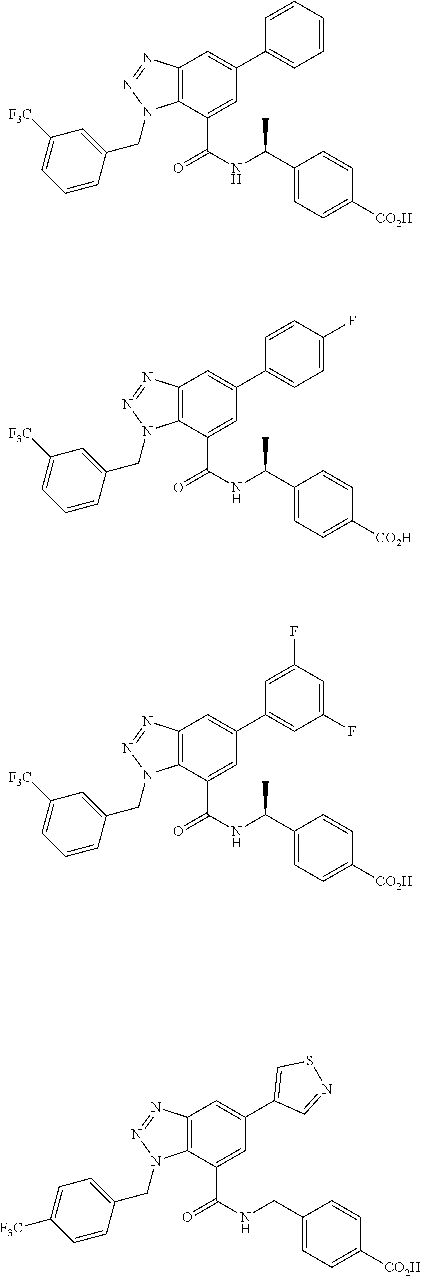 [SMILES: C[C@H](NC(=O)c1cc(-c2ccccc2)cc2nnn(Cc3cccc(C(F)(F)F)c3)c12)c1ccc(C(=O)O)cc1 ; C[C@H](NC(=O)c1cc(-c2ccc(F)cc2)cc2nnn(Cc3cccc(C(F)(F)F)c3)c12)c1ccc(C(=O)O)cc1 ; C[C@H](NC(=O)c1cc(-c2cc(F)cc(F)c2)cc2nnn(Cc3cccc(C(F)(F)F)c3)c12)c1ccc(C(=O)O)cc1 ; O=C(O)c1ccc(CNC(=O)c2cc(-c3cnsc3)cc3nnn(Cc4ccc(C(F)(F)F)cc4)c23)cc1]

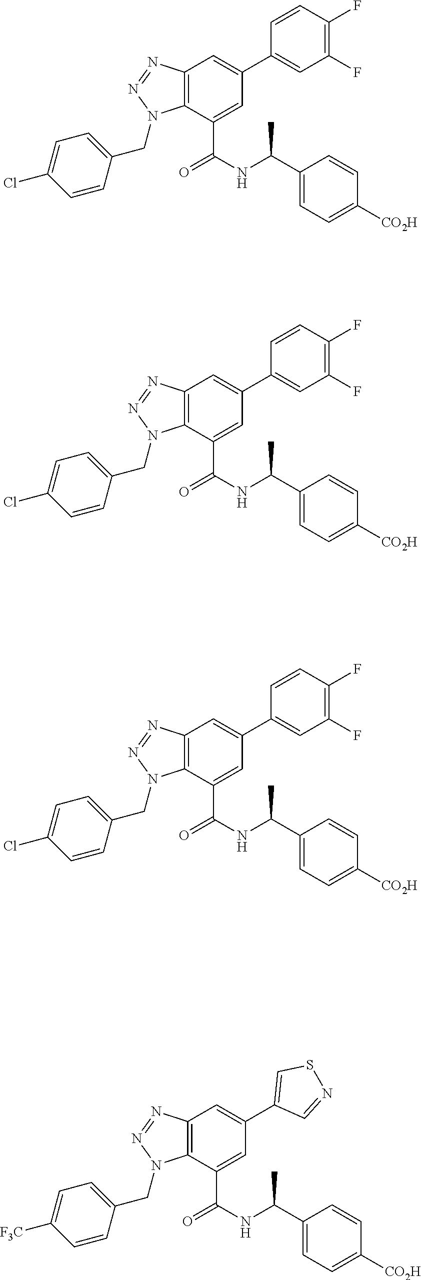 [SMILES: C[C@H](NC(=O)c1cc(-c2ccc(F)c(F)c2)cc2nnn(Cc3ccc(Cl)cc3)c12)c1ccc(C(=O)O)cc1 ; C[C@H](NC(=O)c1cc(-c2ccc(F)c(F)c2)cc2nnn(Cc3ccc(Cl)cc3)c12)c1ccc(C(=O)O)cc1 ; C[C@H](NC(=O)c1cc(-c2ccc(F)c(F)c2)cc2nnn(Cc3ccc(Cl)cc3)c12)c1ccc(C(=O)O)cc1 ; C[C@H](NC(=O)c1cc(-c2cnsc2)cc2nnn(Cc3ccc(C(F)(F)F)cc3)c12)c1ccc(C(=O)O)cc1]

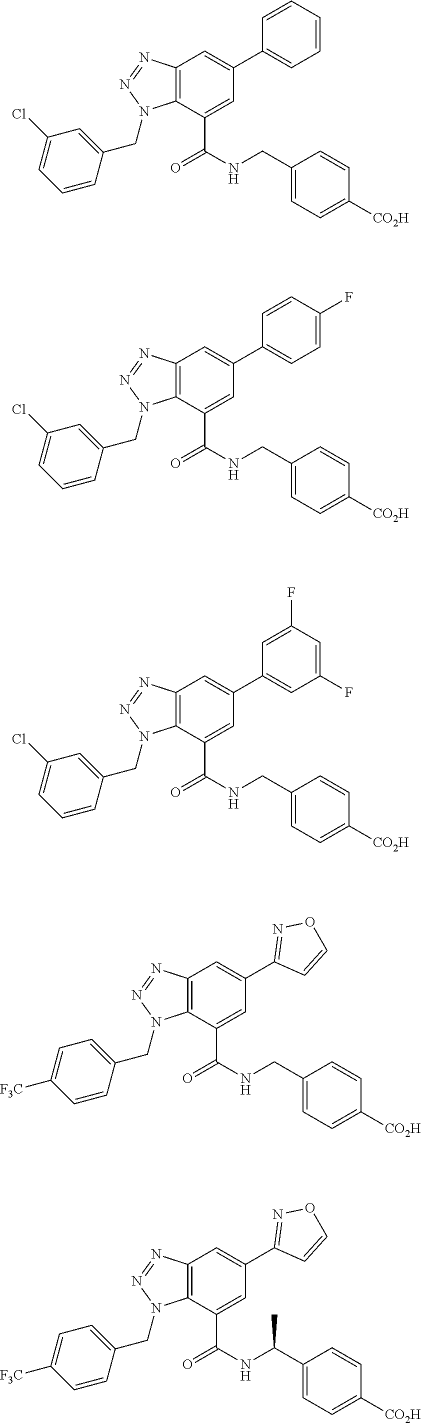 [SMILES: O=C(O)c1ccc(CNC(=O)c2cc(-c3ccccc3)cc3nnn(Cc4cccc(Cl)c4)c23)cc1 ; O=C(O)c1ccc(CNC(=O)c2cc(-c3ccc(F)cc3)cc3nnn(Cc4cccc(Cl)c4)c23)cc1 ; O=C(O)c1ccc(CNC(=O)c2cc(-c3cc(F)cc(F)c3)cc3nnn(Cc4cccc(Cl)c4)c23)cc1 ; O=C(O)c1ccc(CNC(=O)c2cc(-c3ccon3)cc3nnn(Cc4ccc(C(F)(F)F)cc4)c23)cc1 ; C[C@H](NC(=O)c1cc(-c2ccon2)cc2nnn(Cc3ccc(C(F)(F)F)cc3)c12)c1ccc(C(=O)O)cc1]

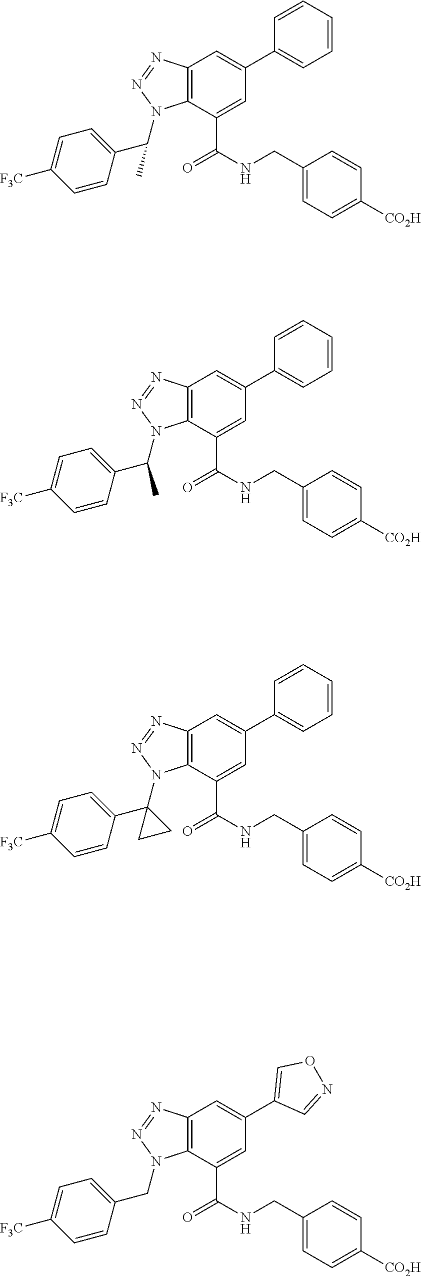 [SMILES: C[C@H](c1ccc(C(F)(F)F)cc1)n1nnc2cc(-c3ccccc3)cc(C(=O)NCc3ccc(C(=O)O)cc3)c21 ; C[C@@H](c1ccc(C(F)(F)F)cc1)n1nnc2cc(-c3ccccc3)cc(C(=O)NCc3ccc(C(=O)O)cc3)c21 ; O=C(O)c1ccc(CNC(=O)c2cc(-c3ccccc3)cc3nnn(C4(c5ccc(C(F)(F)F)cc5)CC4)c23)cc1 ; O=C(O)c1ccc(CNC(=O)c2cc(-c3cnoc3)cc3nnn(Cc4ccc(C(F)(F)F)cc4)c23)cc1]

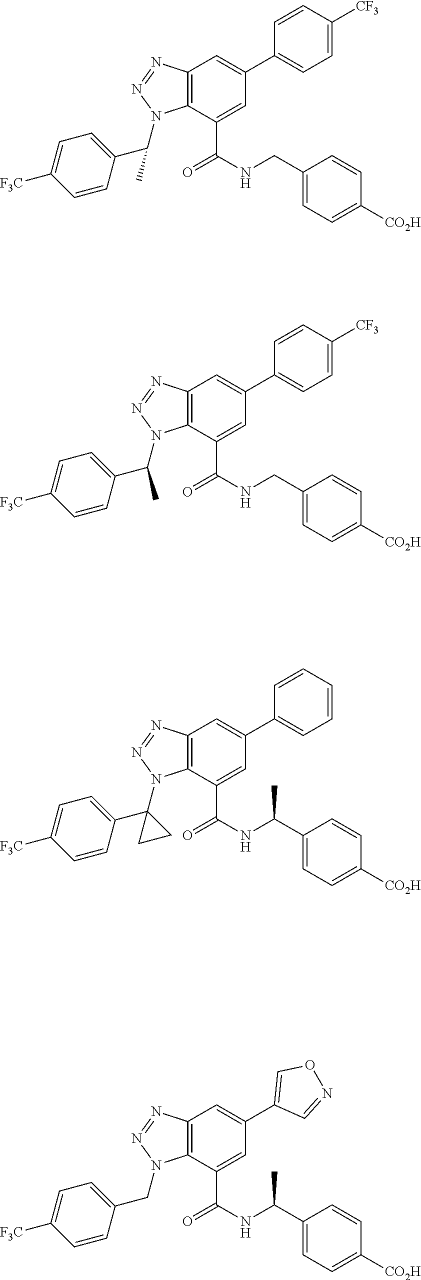 [SMILES: C[C@H](c1ccc(C(F)(F)F)cc1)n1nnc2cc(-c3ccc(C(F)(F)F)cc3)cc(C(=O)NCc3ccc(C(=O)O)cc3)c21 ; C[C@@H](c1ccc(C(F)(F)F)cc1)n1nnc2cc(-c3ccc(C(F)(F)F)cc3)cc(C(=O)NCc3ccc(C(=O)O)cc3)c21 ; C[C@H](NC(=O)c1cc(-c2ccccc2)cc2nnn(C3(c4ccc(C(F)(F)F)cc4)CC3)c12)c1ccc(C(=O)O)cc1 ; C[C@H](NC(=O)c1cc(-c2cnoc2)cc2nnn(Cc3ccc(C(F)(F)F)cc3)c12)c1ccc(C(=O)O)cc1]

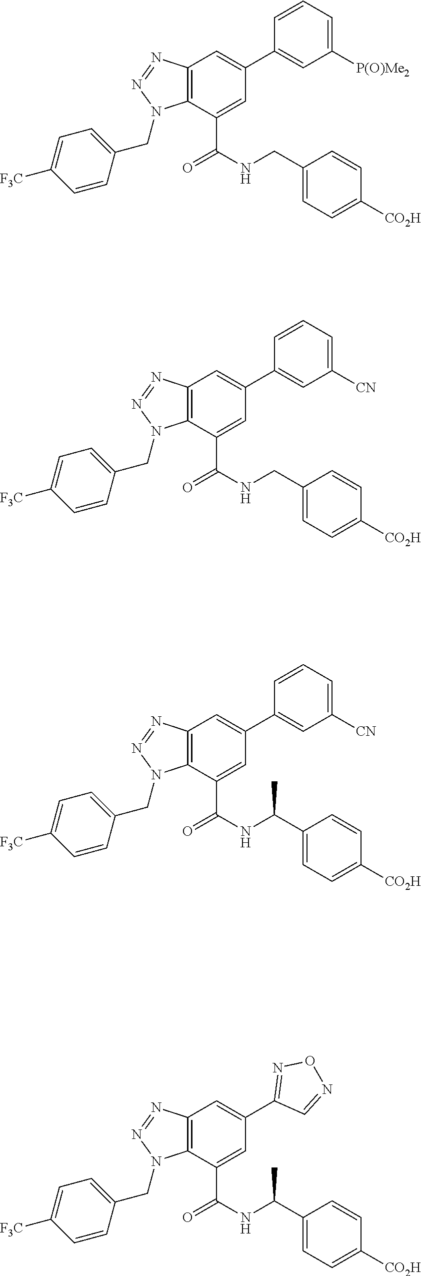

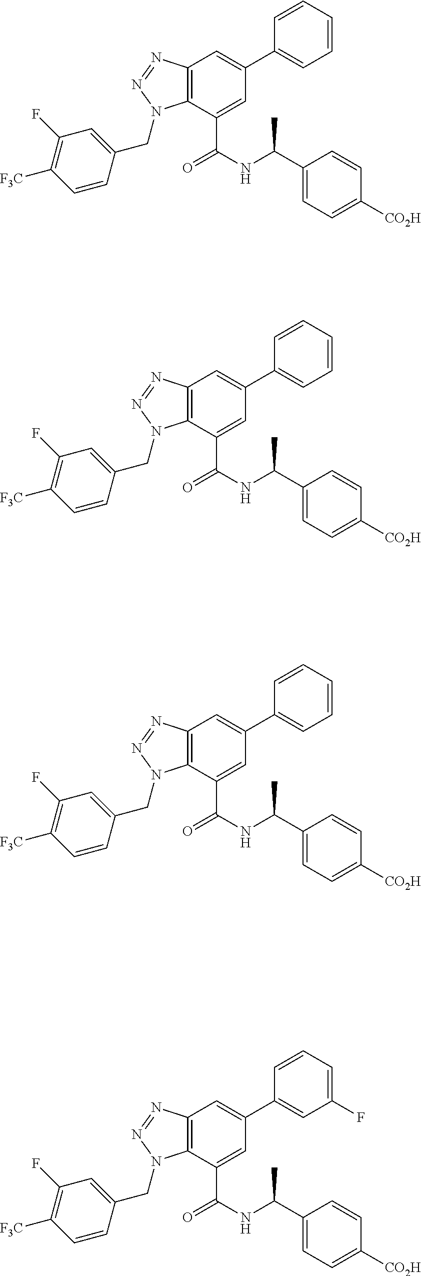 [SMILES: C[C@H](NC(=O)c1cc(-c2ccccc2)cc2nnn(Cc3ccc(C(F)(F)F)c(F)c3)c12)c1ccc(C(=O)O)cc1 ; C[C@H](NC(=O)c1cc(-c2ccccc2)cc2nnn(Cc3ccc(C(F)(F)F)c(F)c3)c12)c1ccc(C(=O)O)cc1 ; C[C@H](NC(=O)c1cc(-c2ccccc2)cc2nnn(Cc3ccc(C(F)(F)F)c(F)c3)c12)c1ccc(C(=O)O)cc1 ; C[C@H](NC(=O)c1cc(-c2cccc(F)c2)cc2nnn(Cc3ccc(C(F)(F)F)c(F)c3)c12)c1ccc(C(=O)O)cc1]

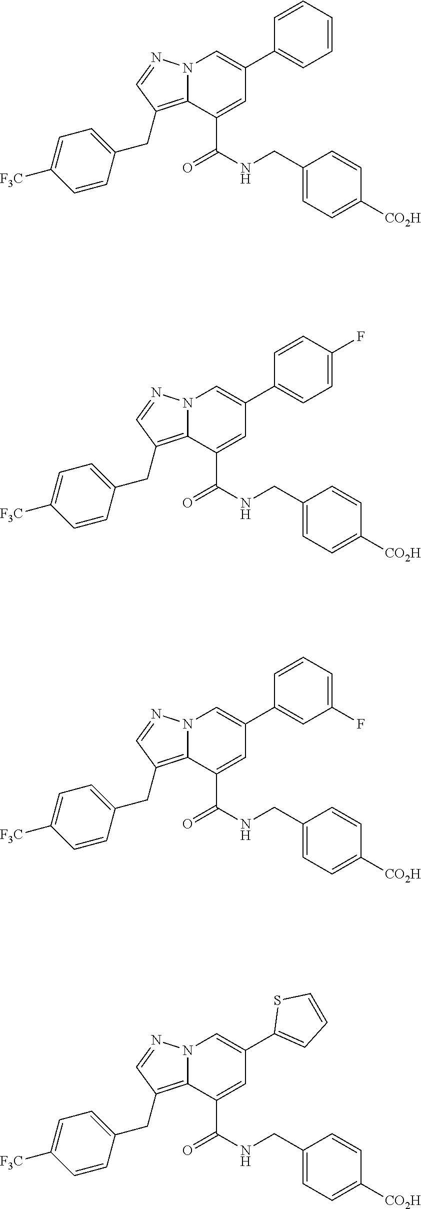 [SMILES: O=C(O)c1ccc(CNC(=O)c2cc(-c3ccccc3)cn3ncc(Cc4ccc(C(F)(F)F)cc4)c23)cc1 ; O=C(O)c1ccc(CNC(=O)c2cc(-c3ccc(F)cc3)cn3ncc(Cc4ccc(C(F)(F)F)cc4)c23)cc1 ; O=C(O)c1ccc(CNC(=O)c2cc(-c3cccc(F)c3)cn3ncc(Cc4ccc(C(F)(F)F)cc4)c23)cc1 ; O=C(O)c1ccc(CNC(=O)c2cc(-c3cccs3)cn3ncc(Cc4ccc(C(F)(F)F)cc4)c23)cc1]

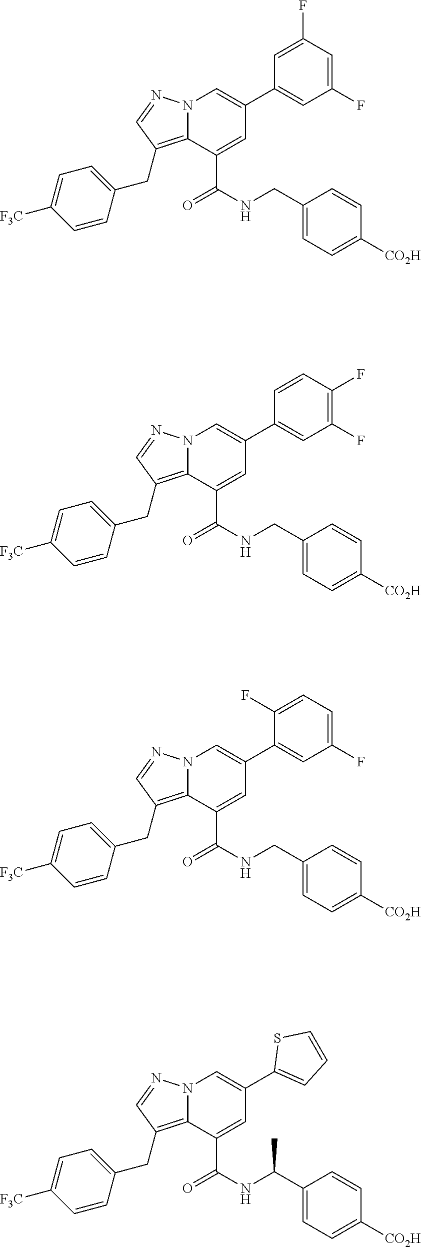 [SMILES: O=C(O)c1ccc(CNC(=O)c2cc(-c3cc(F)cc(F)c3)cn3ncc(Cc4ccc(C(F)(F)F)cc4)c23)cc1 ; O=C(O)c1ccc(CNC(=O)c2cc(-c3ccc(F)c(F)c3)cn3ncc(Cc4ccc(C(F)(F)F)cc4)c23)cc1 ; O=C(O)c1ccc(CNC(=O)c2cc(-c3cc(F)ccc3F)cn3ncc(Cc4ccc(C(F)(F)F)cc4)c23)cc1 ; C[C@H](NC(=O)c1cc(-c2cccs2)cn2ncc(Cc3ccc(C(F)(F)F)cc3)c12)c1ccc(C(=O)O)cc1]

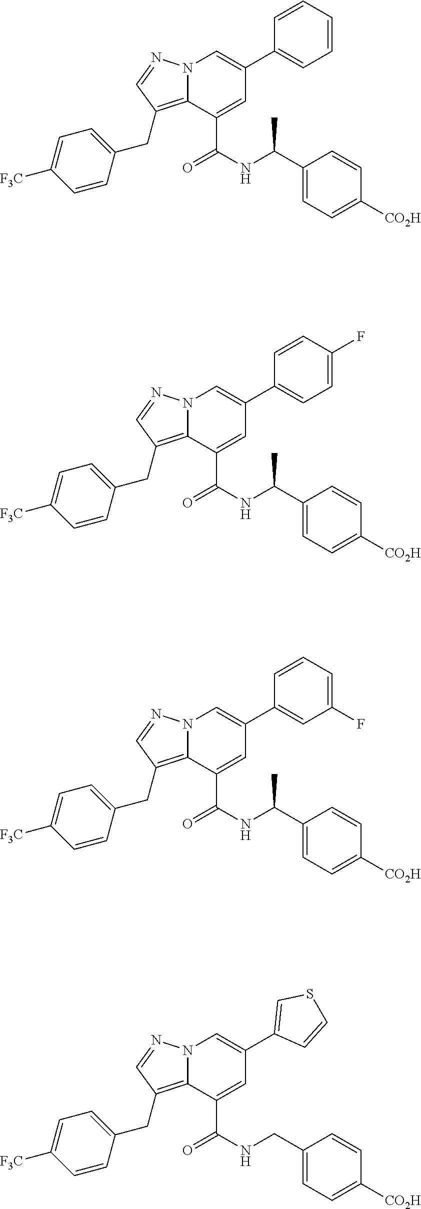 [SMILES: C[C@H](NC(=O)c1cc(-c2ccccc2)cn2ncc(Cc3ccc(C(F)(F)F)cc3)c12)c1ccc(C(=O)O)cc1 ; C[C@H](NC(=O)c1cc(-c2ccc(F)cc2)cn2ncc(Cc3ccc(C(F)(F)F)cc3)c12)c1ccc(C(=O)O)cc1 ; C[C@H](NC(=O)c1cc(-c2cccc(F)c2)cn2ncc(Cc3ccc(C(F)(F)F)cc3)c12)c1ccc(C(=O)O)cc1 ; O=C(O)c1ccc(CNC(=O)c2cc(-c3ccsc3)cn3ncc(Cc4ccc(C(F)(F)F)cc4)c23)cc1]

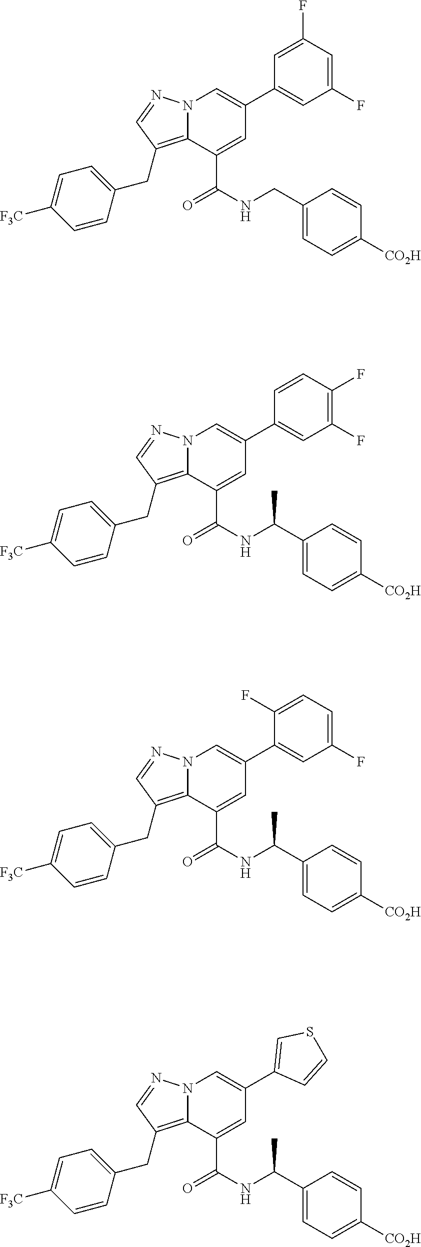 [SMILES: O=C(O)c1ccc(CNC(=O)c2cc(-c3cc(F)cc(F)c3)cn3ncc(Cc4ccc(C(F)(F)F)cc4)c23)cc1 ; C[C@H](NC(=O)c1cc(-c2ccc(F)c(F)c2)cn2ncc(Cc3ccc(C(F)(F)F)cc3)c12)c1ccc(C(=O)O)cc1 ; C[C@H](NC(=O)c1cc(-c2cc(F)ccc2F)cn2ncc(Cc3ccc(C(F)(F)F)cc3)c12)c1ccc(C(=O)O)cc1 ; C[C@H](NC(=O)c1cc(-c2ccsc2)cn2ncc(Cc3ccc(C(F)(F)F)cc3)c12)c1ccc(C(=O)O)cc1]

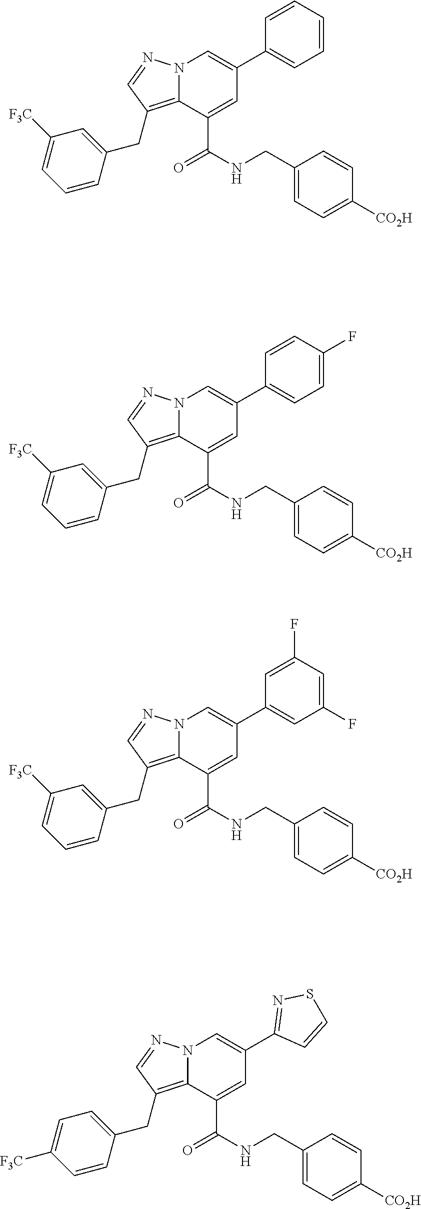 [SMILES: O=C(O)c1ccc(CNC(=O)c2cc(-c3ccccc3)cn3ncc(Cc4cccc(C(F)(F)F)c4)c23)cc1 ; O=C(O)c1ccc(CNC(=O)c2cc(-c3ccc(F)cc3)cn3ncc(Cc4cccc(C(F)(F)F)c4)c23)cc1 ; O=C(O)c1ccc(CNC(=O)c2cc(-c3cc(F)cc(F)c3)cn3ncc(Cc4cccc(C(F)(F)F)c4)c23)cc1 ; O=C(O)c1ccc(CNC(=O)c2cc(-c3ccsn3)cn3ncc(Cc4ccc(C(F)(F)F)cc4)c23)cc1]

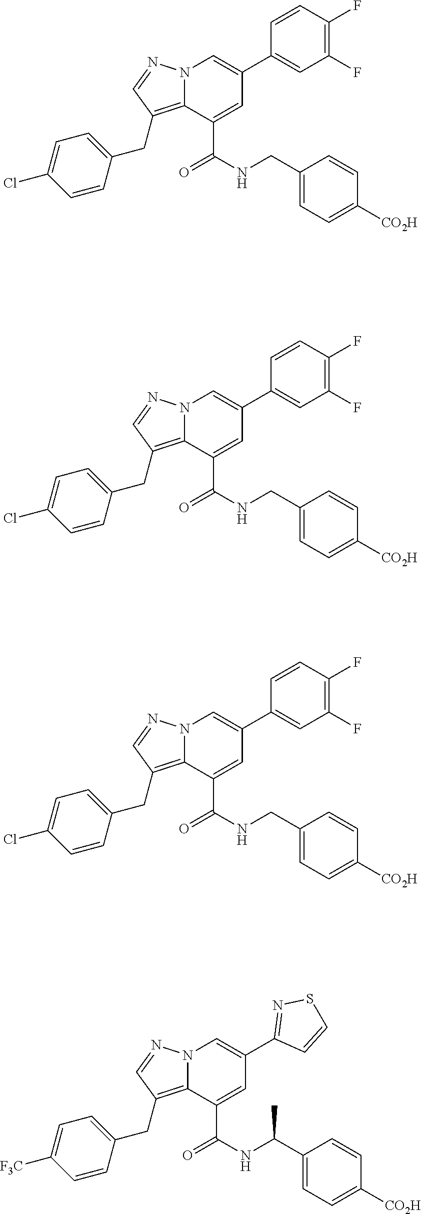 [SMILES: O=C(O)c1ccc(CNC(=O)c2cc(-c3ccc(F)c(F)c3)cn3ncc(Cc4ccc(Cl)cc4)c23)cc1 ; O=C(O)c1ccc(CNC(=O)c2cc(-c3ccc(F)c(F)c3)cn3ncc(Cc4ccc(Cl)cc4)c23)cc1 ; O=C(O)c1ccc(CNC(=O)c2cc(-c3ccc(F)c(F)c3)cn3ncc(Cc4ccc(Cl)cc4)c23)cc1 ; C[C@H](NC(=O)c1cc(-c2ccsn2)cn2ncc(Cc3ccc(C(F)(F)F)cc3)c12)c1ccc(C(=O)O)cc1]

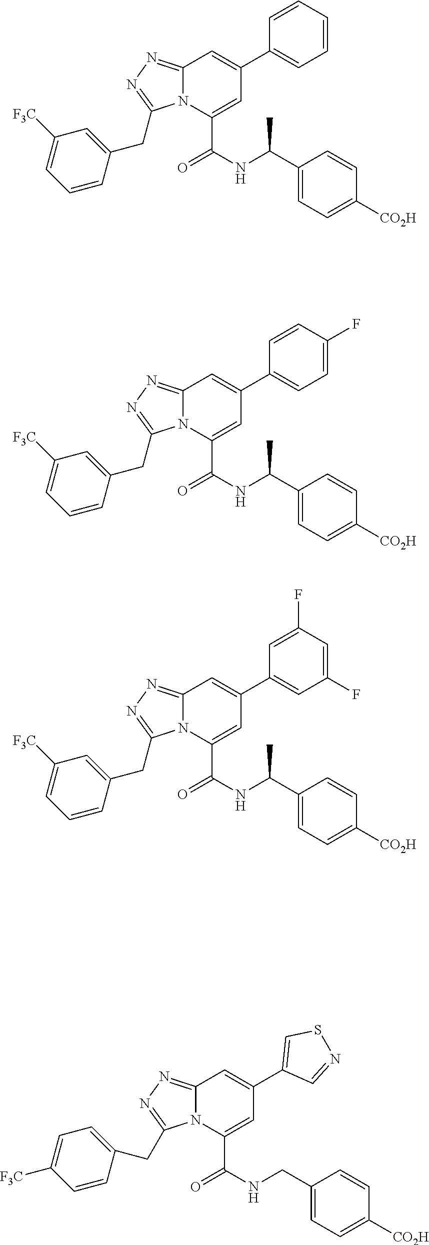 [SMILES: C[C@H](NC(=O)c1cc(-c2ccccc2)cc2nnc(Cc3cccc(C(F)(F)F)c3)n12)c1ccc(C(=O)O)cc1 ; C[C@H](NC(=O)c1cc(-c2ccc(F)cc2)cc2nnc(Cc3cccc(C(F)(F)F)c3)n12)c1ccc(C(=O)O)cc1 ; C[C@H](NC(=O)c1cc(-c2cc(F)cc(F)c2)cc2nnc(Cc3cccc(C(F)(F)F)c3)n12)c1ccc(C(=O)O)cc1 ; O=C(O)c1ccc(CNC(=O)c2cc(-c3cnsc3)cc3nnc(Cc4ccc(C(F)(F)F)cc4)n23)cc1]

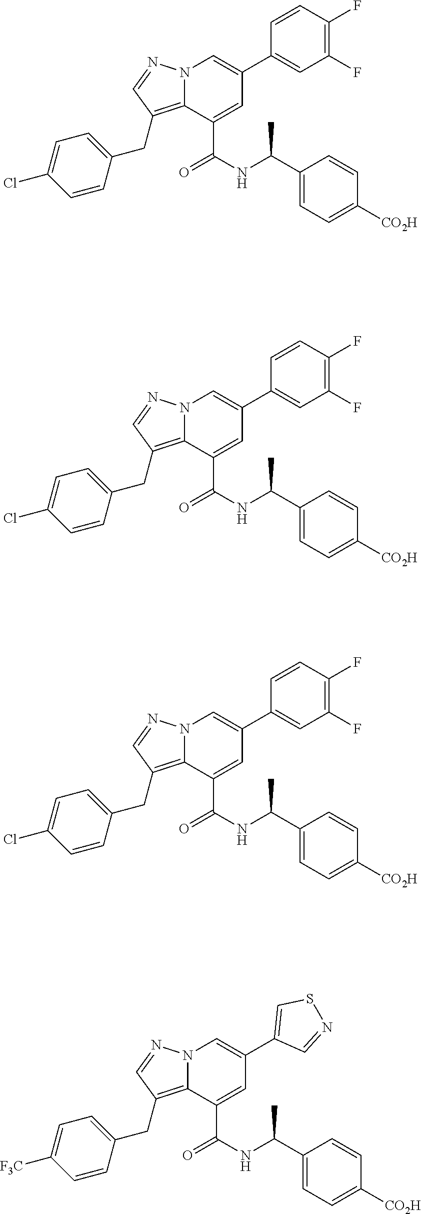 [SMILES: C[C@H](NC(=O)c1cc(-c2ccc(F)c(F)c2)cn2ncc(Cc3ccc(Cl)cc3)c12)c1ccc(C(=O)O)cc1 ; C[C@H](NC(=O)c1cc(-c2ccc(F)c(F)c2)cn2ncc(Cc3ccc(Cl)cc3)c12)c1ccc(C(=O)O)cc1 ; C[C@H](NC(=O)c1cc(-c2ccc(F)c(F)c2)cn2ncc(Cc3ccc(Cl)cc3)c12)c1ccc(C(=O)O)cc1 ; C[C@H](NC(=O)c1cc(-c2cnsc2)cn2ncc(Cc3ccc(C(F)(F)F)cc3)c12)c1ccc(C(=O)O)cc1]

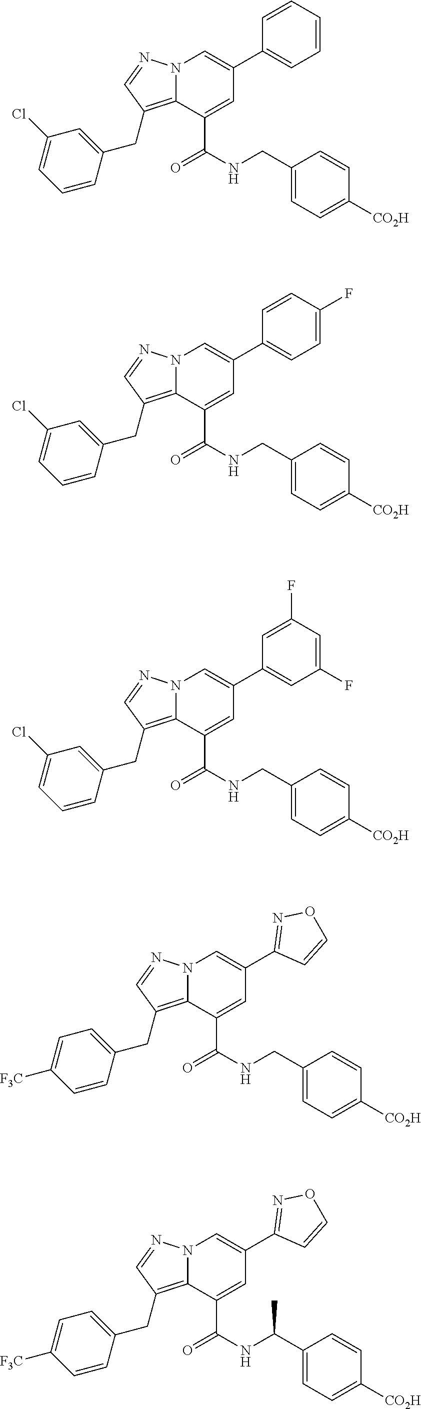 [SMILES: O=C(O)c1ccc(CNC(=O)c2cc(-c3ccccc3)cn3ncc(Cc4cccc(Cl)c4)c23)cc1 ; O=C(O)c1ccc(CNC(=O)c2cc(-c3ccc(F)cc3)cn3ncc(Cc4cccc(Cl)c4)c23)cc1 ; O=C(O)c1ccc(CNC(=O)c2cc(-c3cc(F)cc(F)c3)cn3ncc(Cc4cccc(Cl)c4)c23)cc1 ; O=C(O)c1ccc(CNC(=O)c2cc(-c3ccon3)cn3ncc(Cc4ccc(C(F)(F)F)cc4)c23)cc1 ; C[C@H](NC(=O)c1cc(-c2ccon2)cn2ncc(Cc3ccc(C(F)(F)F)cc3)c12)c1ccc(C(=O)O)cc1]

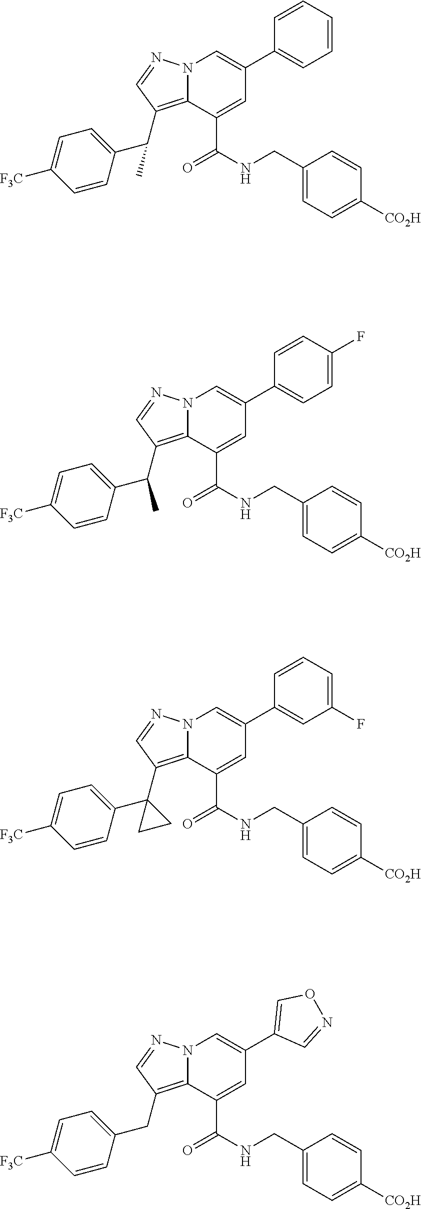 [SMILES: C[C@H](c1ccc(C(F)(F)F)cc1)c1cnn2cc(-c3ccccc3)cc(C(=O)NCc3ccc(C(=O)O)cc3)c12 ; C[C@@H](c1ccc(C(F)(F)F)cc1)c1cnn2cc(-c3ccc(F)cc3)cc(C(=O)NCc3ccc(C(=O)O)cc3)c12 ; O=C(O)c1ccc(CNC(=O)c2cc(-c3cccc(F)c3)cn3ncc(C4(c5ccc(C(F)(F)F)cc5)CC4)c23)cc1 ; O=C(O)c1ccc(CNC(=O)c2cc(-c3cnoc3)cn3ncc(Cc4ccc(C(F)(F)F)cc4)c23)cc1]

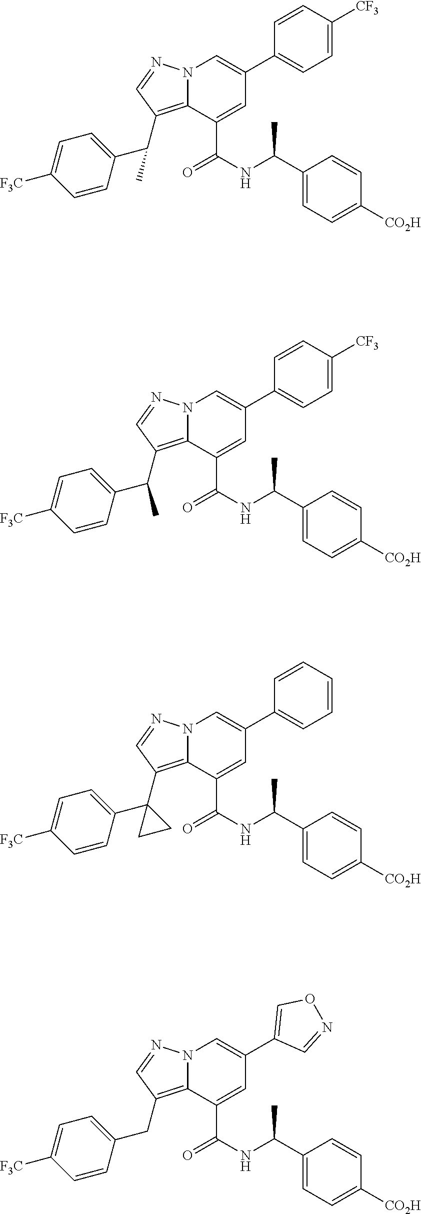 [SMILES: C[C@H](c1ccc(C(F)(F)F)cc1)c1cnn2cc(-c3ccc(C(F)(F)F)cc3)cc(C(=O)N[C@@H](C)c3ccc(C(=O)O)cc3)c12 ; C[C@@H](c1ccc(C(F)(F)F)cc1)c1cnn2cc(-c3ccc(C(F)(F)F)cc3)cc(C(=O)N[C@@H](C)c3ccc(C(=O)O)cc3)c12 ; C[C@H](NC(=O)c1cc(-c2ccccc2)cn2ncc(C3(c4ccc(C(F)(F)F)cc4)CC3)c12)c1ccc(C(=O)O)cc1 ; C[C@H](NC(=O)c1cc(-c2cnoc2)cn2ncc(Cc3ccc(C(F)(F)F)cc3)c12)c1ccc(C(=O)O)cc1]

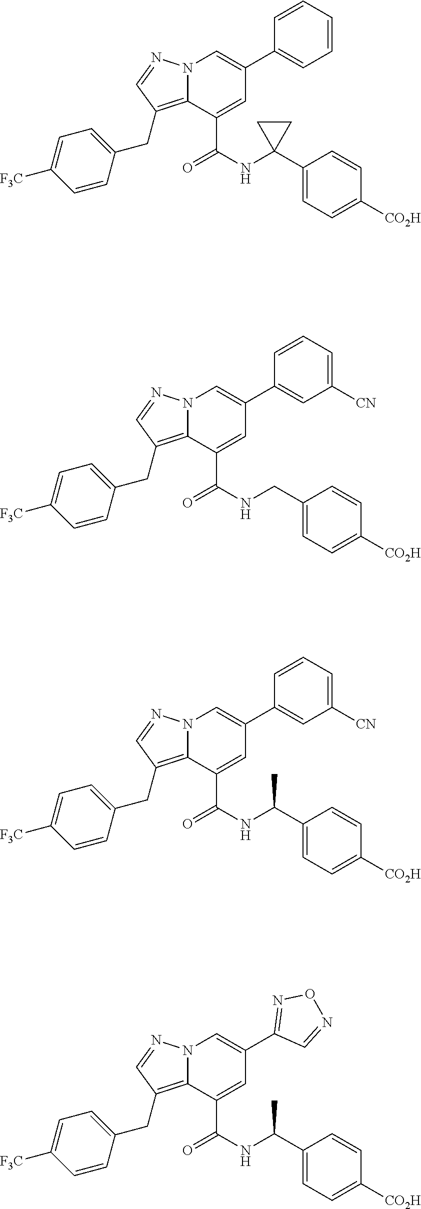 [SMILES: O=C(O)c1ccc(C2(NC(=O)c3cc(-c4ccccc4)cn4ncc(Cc5ccc(C(F)(F)F)cc5)c34)CC2)cc1 ; N#Cc1cccc(-c2cc(C(=O)NCc3ccc(C(=O)O)cc3)c3c(Cc4ccc(C(F)(F)F)cc4)cnn3c2)c1 ; C[C@H](NC(=O)c1cc(-c2cccc(C#N)c2)cn2ncc(Cc3ccc(C(F)(F)F)cc3)c12)c1ccc(C(=O)O)cc1 ; C[C@H](NC(=O)c1cc(-c2cnon2)cn2ncc(Cc3ccc(C(F)(F)F)cc3)c12)c1ccc(C(=O)O)cc1]

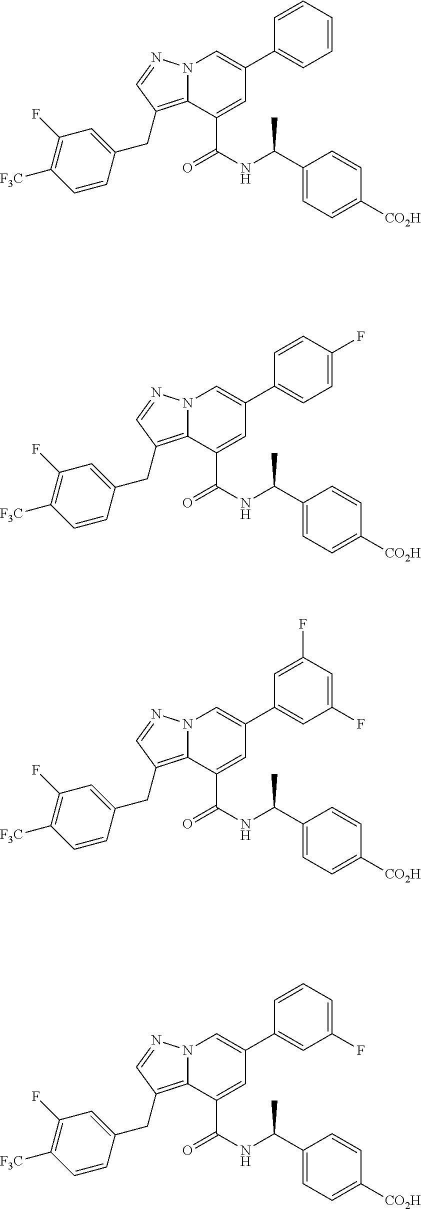 [SMILES: C[C@H](NC(=O)c1cc(-c2ccccc2)cn2ncc(Cc3ccc(C(F)(F)F)c(F)c3)c12)c1ccc(C(=O)O)cc1 ; C[C@H](NC(=O)c1cc(-c2ccc(F)cc2)cn2ncc(Cc3ccc(C(F)(F)F)c(F)c3)c12)c1ccc(C(=O)O)cc1 ; C[C@H](NC(=O)c1cc(-c2cc(F)cc(F)c2)cn2ncc(Cc3ccc(C(F)(F)F)c(F)c3)c12)c1ccc(C(=O)O)cc1 ; C[C@H](NC(=O)c1cc(-c2cccc(F)c2)cn2ncc(Cc3ccc(C(F)(F)F)c(F)c3)c12)c1ccc(C(=O)O)cc1]

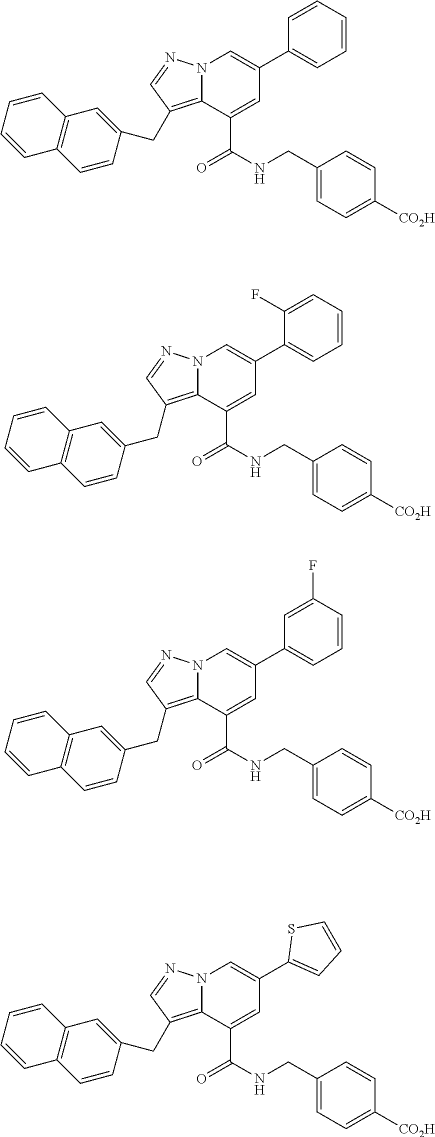 [SMILES: O=C(O)c1ccc(CNC(=O)c2cc(-c3ccccc3)cn3ncc(Cc4ccc5ccccc5c4)c23)cc1 ; O=C(O)c1ccc(CNC(=O)c2cc(-c3ccccc3F)cn3ncc(Cc4ccc5ccccc5c4)c23)cc1 ; O=C(O)c1ccc(CNC(=O)c2cc(-c3cccc(F)c3)cn3ncc(Cc4ccc5ccccc5c4)c23)cc1 ; O=C(O)c1ccc(CNC(=O)c2cc(-c3cccs3)cn3ncc(Cc4ccc5ccccc5c4)c23)cc1]

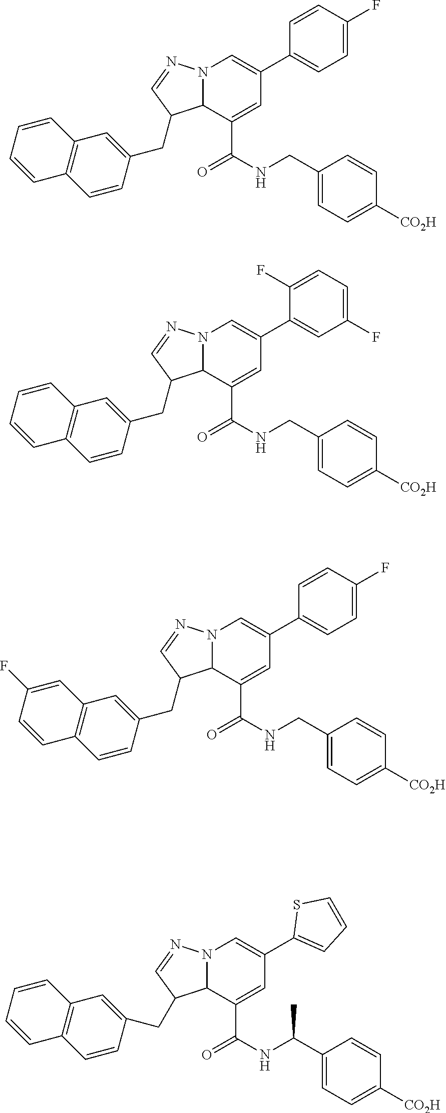 [SMILES: O=C(NCc1ccc(C(=O)O)cc1)C1=CC(c2ccc(F)cc2)=CN2N=CC(Cc3ccc4ccccc4c3)C12 ; O=C(NCc1ccc(C(=O)O)cc1)C1=CC(c2cc(F)ccc2F)=CN2N=CC(Cc3ccc4ccccc4c3)C12 ; O=C(NCc1ccc(C(=O)O)cc1)C1=CC(c2ccc(F)cc2)=CN2N=CC(Cc3ccc4ccc(F)cc4c3)C12 ; C[C@H](NC(=O)C1=CC(c2cccs2)=CN2N=CC(Cc3ccc4ccccc4c3)C12)c1ccc(C(=O)O)cc1]

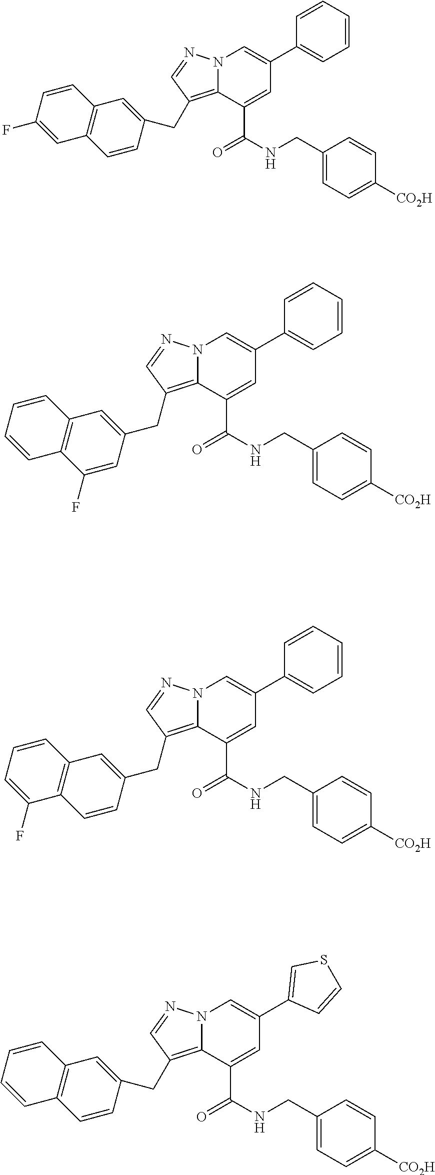 [SMILES: O=C(O)c1ccc(CNC(=O)c2cc(-c3ccccc3)cn3ncc(Cc4ccc5cc(F)ccc5c4)c23)cc1 ; O=C(O)c1ccc(CNC(=O)c2cc(-c3ccccc3)cn3ncc(Cc4cc(F)c5ccccc5c4)c23)cc1 ; O=C(O)c1ccc(CNC(=O)c2cc(-c3ccccc3)cn3ncc(Cc4ccc5c(F)cccc5c4)c23)cc1 ; O=C(O)c1ccc(CNC(=O)c2cc(-c3ccsc3)cn3ncc(Cc4ccc5ccccc5c4)c23)cc1]

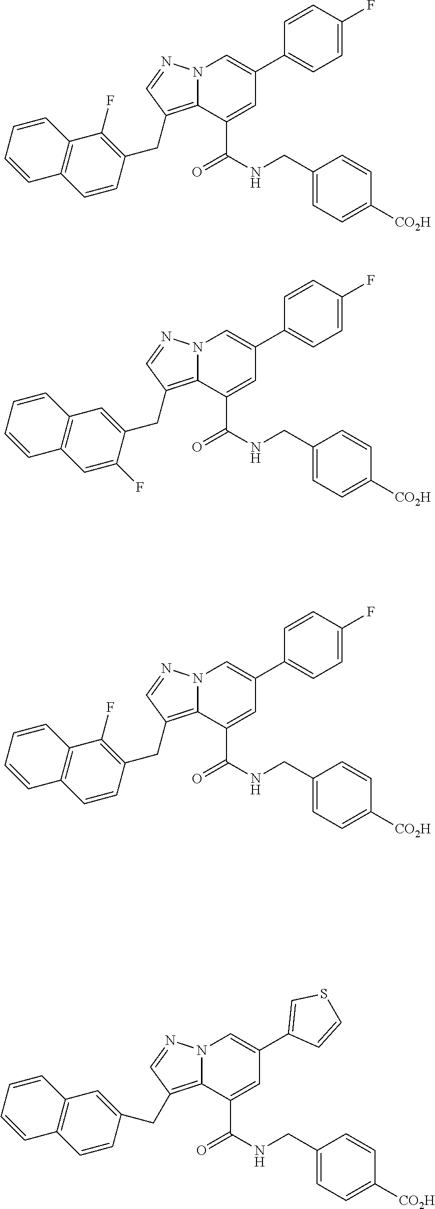 [SMILES: O=C(O)c1ccc(CNC(=O)c2cc(-c3ccc(F)cc3)cn3ncc(Cc4ccc5ccccc5c4F)c23)cc1 ; O=C(O)c1ccc(CNC(=O)c2cc(-c3ccc(F)cc3)cn3ncc(Cc4cc5ccccc5cc4F)c23)cc1 ; O=C(O)c1ccc(CNC(=O)c2cc(-c3ccc(F)cc3)cn3ncc(Cc4ccc5ccccc5c4F)c23)cc1 ; O=C(O)c1ccc(CNC(=O)c2cc(-c3ccsc3)cn3ncc(Cc4ccc5ccccc5c4)c23)cc1]

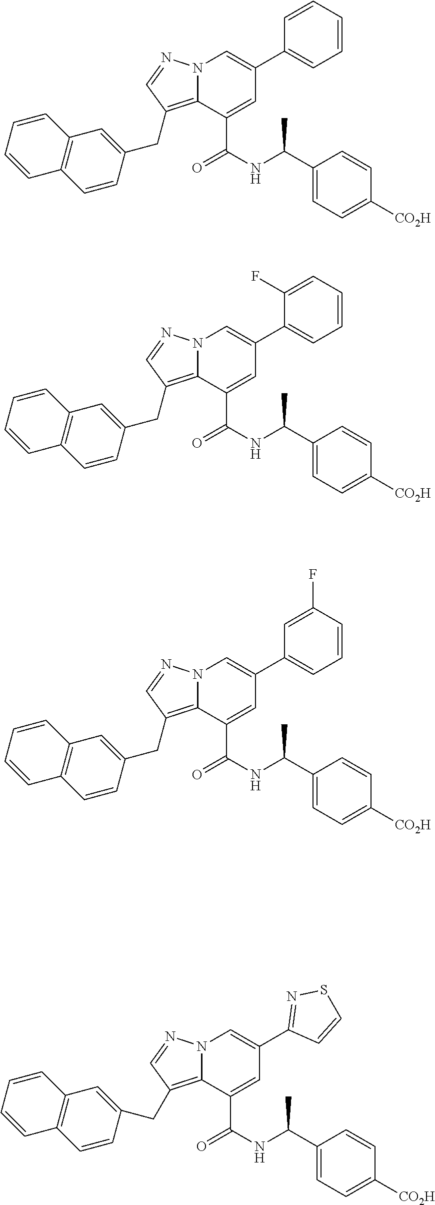 [SMILES: C[C@H](NC(=O)c1cc(-c2ccccc2)cn2ncc(Cc3ccc4ccccc4c3)c12)c1ccc(C(=O)O)cc1 ; C[C@H](NC(=O)c1cc(-c2ccccc2F)cn2ncc(Cc3ccc4ccccc4c3)c12)c1ccc(C(=O)O)cc1 ; C[C@H](NC(=O)c1cc(-c2cccc(F)c2)cn2ncc(Cc3ccc4ccccc4c3)c12)c1ccc(C(=O)O)cc1 ; C[C@H](NC(=O)c1cc(-c2ccsn2)cn2ncc(Cc3ccc4ccccc4c3)c12)c1ccc(C(=O)O)cc1]

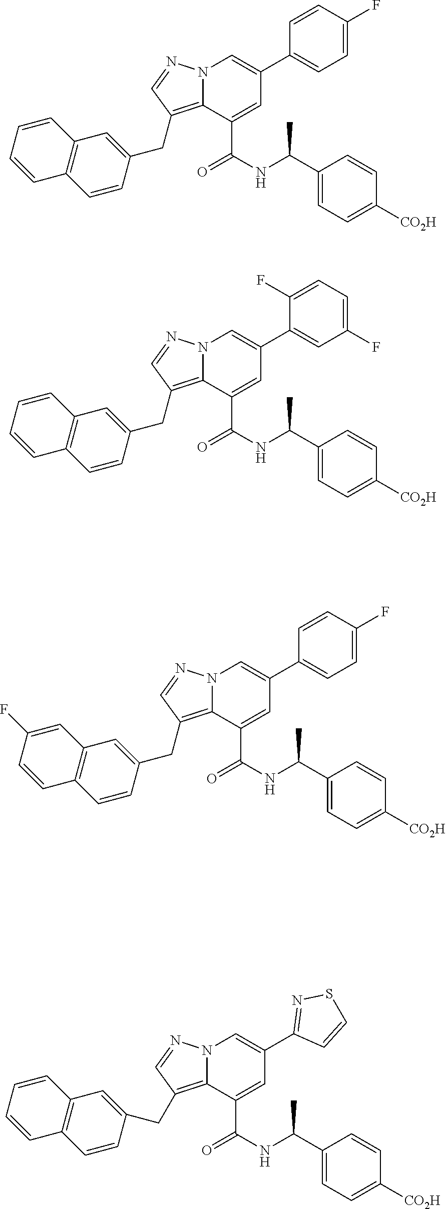 [SMILES: C[C@H](NC(=O)c1cc(-c2ccc(F)cc2)cn2ncc(Cc3ccc4ccccc4c3)c12)c1ccc(C(=O)O)cc1 ; C[C@H](NC(=O)c1cc(-c2cc(F)ccc2F)cn2ncc(Cc3ccc4ccccc4c3)c12)c1ccc(C(=O)O)cc1 ; C[C@H](NC(=O)c1cc(-c2ccc(F)cc2)cn2ncc(Cc3ccc4ccc(F)cc4c3)c12)c1ccc(C(=O)O)cc1 ; C[C@H](NC(=O)c1cc(-c2ccsn2)cn2ncc(Cc3ccc4ccccc4c3)c12)c1ccc(C(=O)O)cc1]

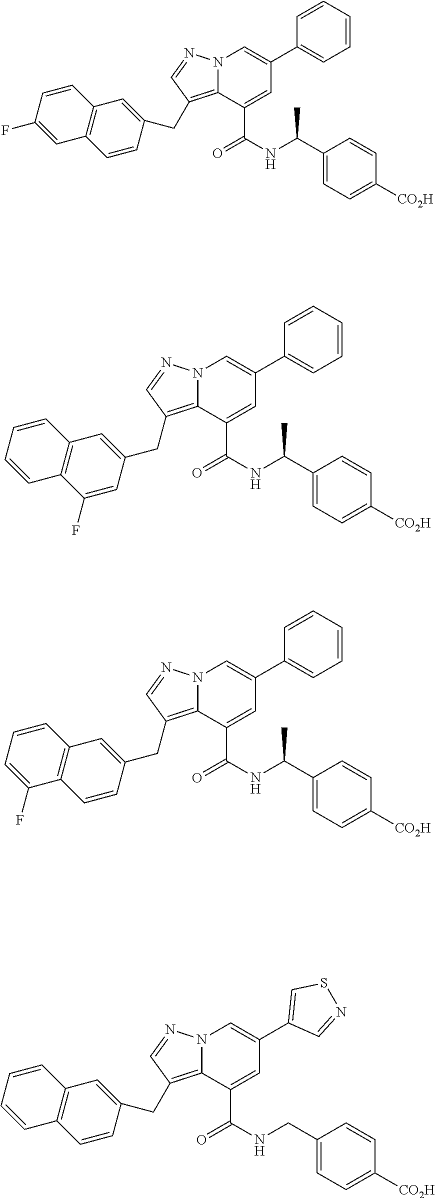 [SMILES: C[C@H](NC(=O)c1cc(-c2ccccc2)cn2ncc(Cc3ccc4cc(F)ccc4c3)c12)c1ccc(C(=O)O)cc1 ; C[C@H](NC(=O)c1cc(-c2ccccc2)cn2ncc(Cc3cc(F)c4ccccc4c3)c12)c1ccc(C(=O)O)cc1 ; C[C@H](NC(=O)c1cc(-c2ccccc2)cn2ncc(Cc3ccc4c(F)cccc4c3)c12)c1ccc(C(=O)O)cc1 ; O=C(O)c1ccc(CNC(=O)c2cc(-c3cnsc3)cn3ncc(Cc4ccc5ccccc5c4)c23)cc1]

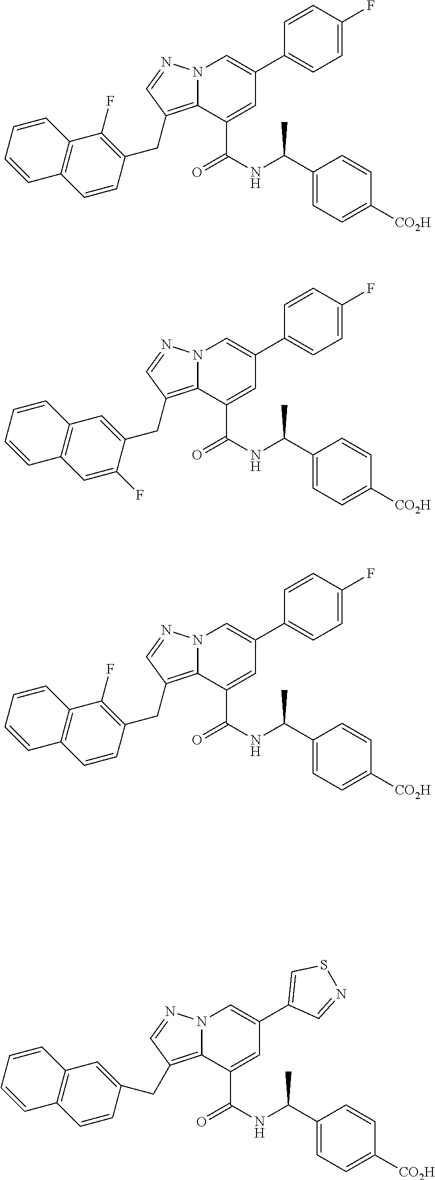 [SMILES: C[C@H](NC(=O)c1cc(-c2ccc(F)cc2)cn2ncc(Cc3ccc4ccccc4c3F)c12)c1ccc(C(=O)O)cc1 ; C[C@H](NC(=O)c1cc(-c2ccc(F)cc2)cn2ncc(Cc3cc4ccccc4cc3F)c12)c1ccc(C(=O)O)cc1 ; C[C@H](NC(=O)c1cc(-c2ccc(F)cc2)cn2ncc(Cc3ccc4ccccc4c3F)c12)c1ccc(C(=O)O)cc1 ; C[C@H](NC(=O)c1cc(-c2cnsc2)cn2ncc(Cc3ccc4ccccc4c3)c12)c1ccc(C(=O)O)cc1]

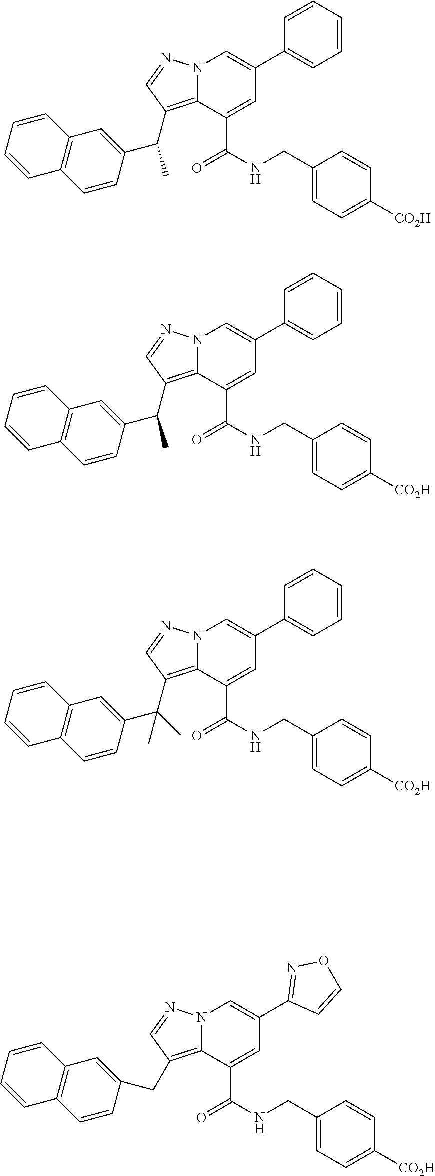 [SMILES: C[C@H](c1ccc2ccccc2c1)c1cnn2cc(-c3ccccc3)cc(C(=O)NCc3ccc(C(=O)O)cc3)c12 ; C[C@@H](c1ccc2ccccc2c1)c1cnn2cc(-c3ccccc3)cc(C(=O)NCc3ccc(C(=O)O)cc3)c12 ; CC(C)(c1ccc2ccccc2c1)c1cnn2cc(-c3ccccc3)cc(C(=O)NCc3ccc(C(=O)O)cc3)c12 ; O=C(O)c1ccc(CNC(=O)c2cc(-c3ccon3)cn3ncc(Cc4ccc5ccccc5c4)c23)cc1]

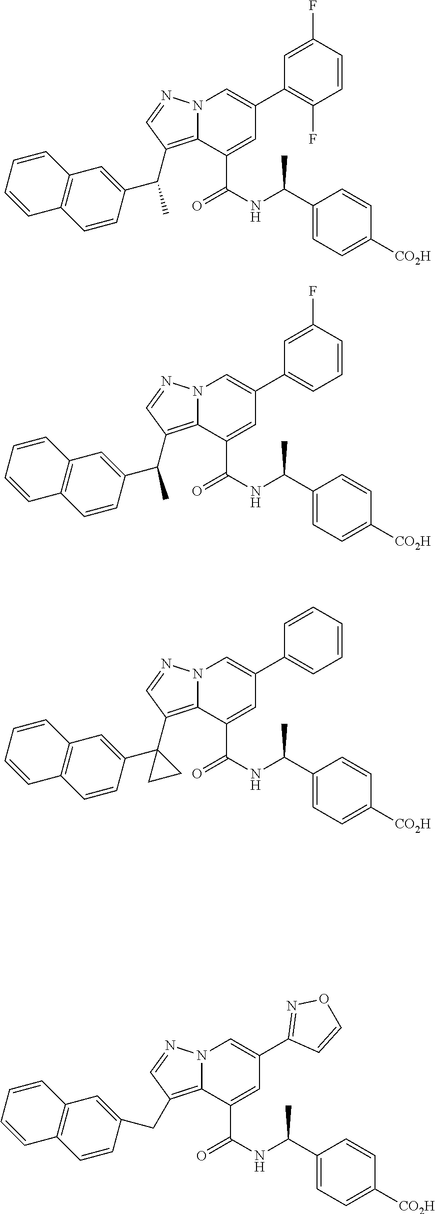

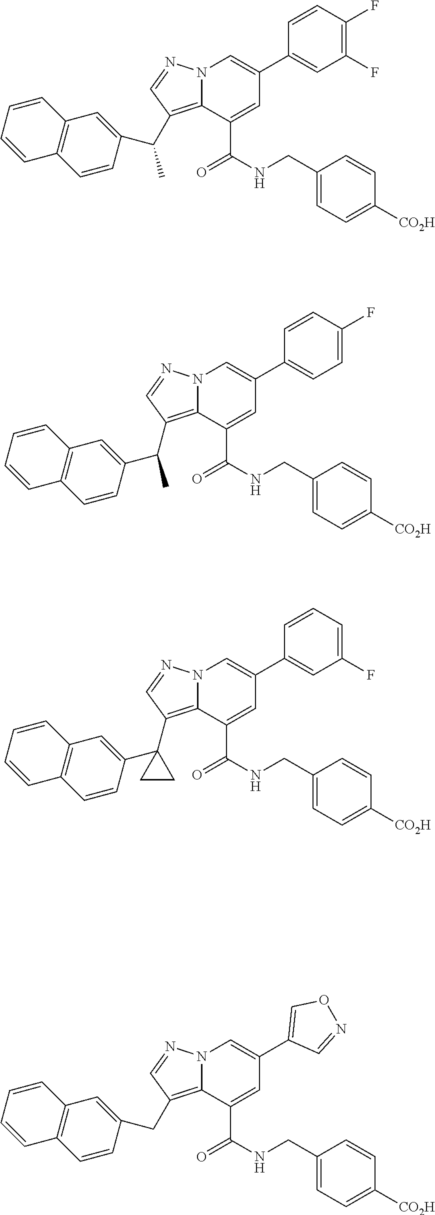 [SMILES: C[C@H](c1ccc2ccccc2c1)c1cnn2cc(-c3ccc(F)c(F)c3)cc(C(=O)NCc3ccc(C(=O)O)cc3)c12 ; C[C@@H](c1ccc2ccccc2c1)c1cnn2cc(-c3ccc(F)cc3)cc(C(=O)NCc3ccc(C(=O)O)cc3)c12 ; O=C(O)c1ccc(CNC(=O)c2cc(-c3cccc(F)c3)cn3ncc(C4(c5ccc6ccccc6c5)CC4)c23)cc1 ; O=C(O)c1ccc(CNC(=O)c2cc(-c3cnoc3)cn3ncc(Cc4ccc5ccccc5c4)c23)cc1]

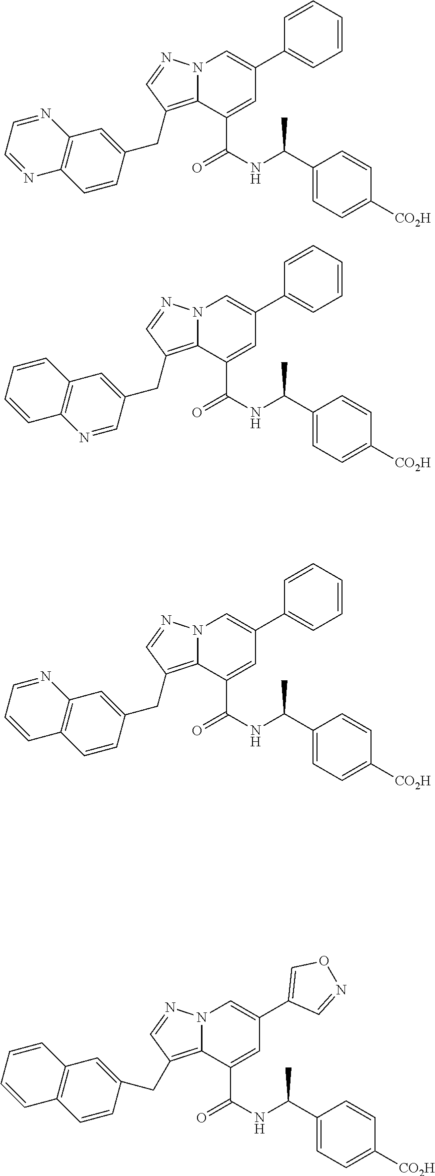

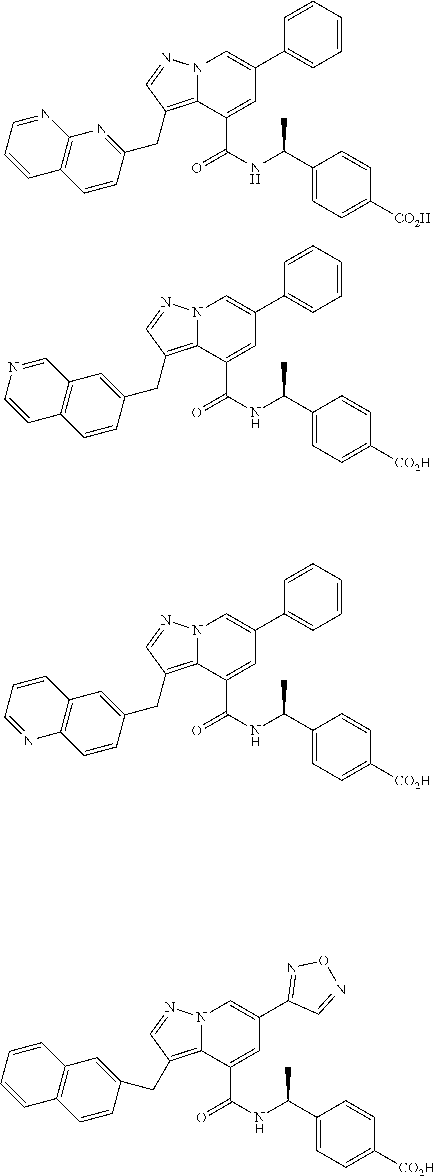

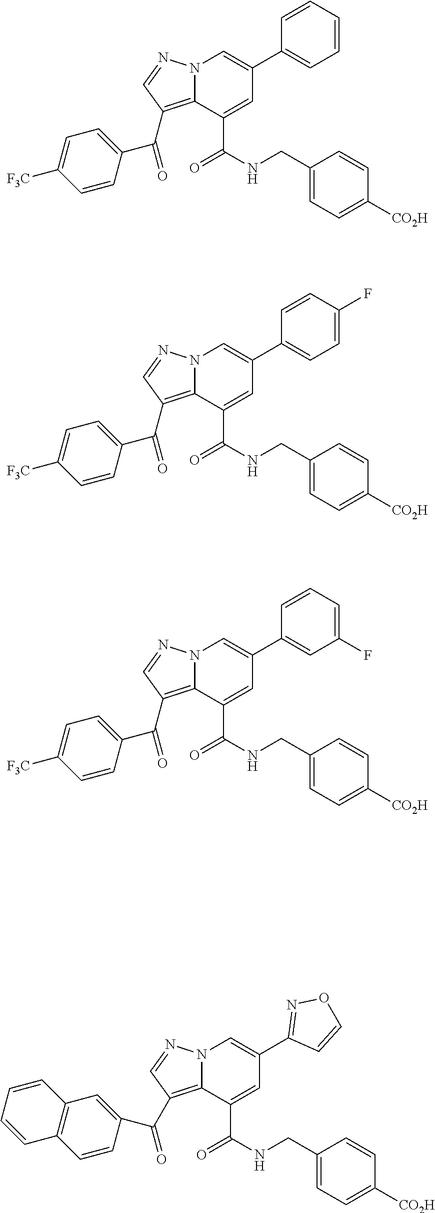 [SMILES: O=C(O)c1ccc(CNC(=O)c2cc(-c3ccccc3)cn3ncc(C(=O)c4ccc(C(F)(F)F)cc4)c23)cc1 ; O=C(O)c1ccc(CNC(=O)c2cc(-c3ccc(F)cc3)cn3ncc(C(=O)c4ccc(C(F)(F)F)cc4)c23)cc1 ; O=C(O)c1ccc(CNC(=O)c2cc(-c3cccc(F)c3)cn3ncc(C(=O)c4ccc(C(F)(F)F)cc4)c23)cc1 ; O=C(O)c1ccc(CNC(=O)c2cc(-c3ccon3)cn3ncc(C(=O)c4ccc5ccccc5c4)c23)cc1]

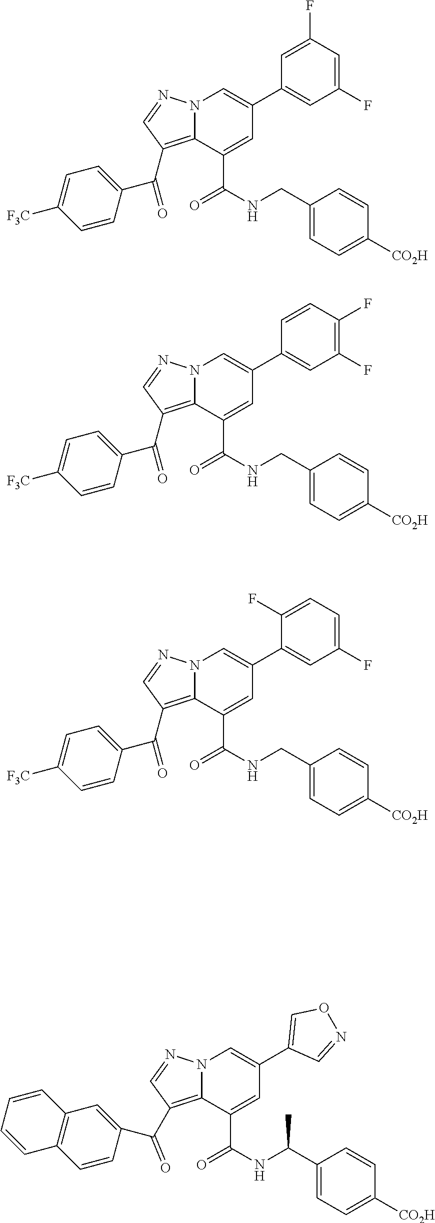 [SMILES: O=C(O)c1ccc(CNC(=O)c2cc(-c3cc(F)cc(F)c3)cn3ncc(C(=O)c4ccc(C(F)(F)F)cc4)c23)cc1 ; O=C(O)c1ccc(CNC(=O)c2cc(-c3ccc(F)c(F)c3)cn3ncc(C(=O)c4ccc(C(F)(F)F)cc4)c23)cc1 ; O=C(O)c1ccc(CNC(=O)c2cc(-c3cc(F)ccc3F)cn3ncc(C(=O)c4ccc(C(F)(F)F)cc4)c23)cc1 ; C[C@H](NC(=O)c1cc(-c2cnoc2)cn2ncc(C(=O)c3ccc4ccccc4c3)c12)c1ccc(C(=O)O)cc1]

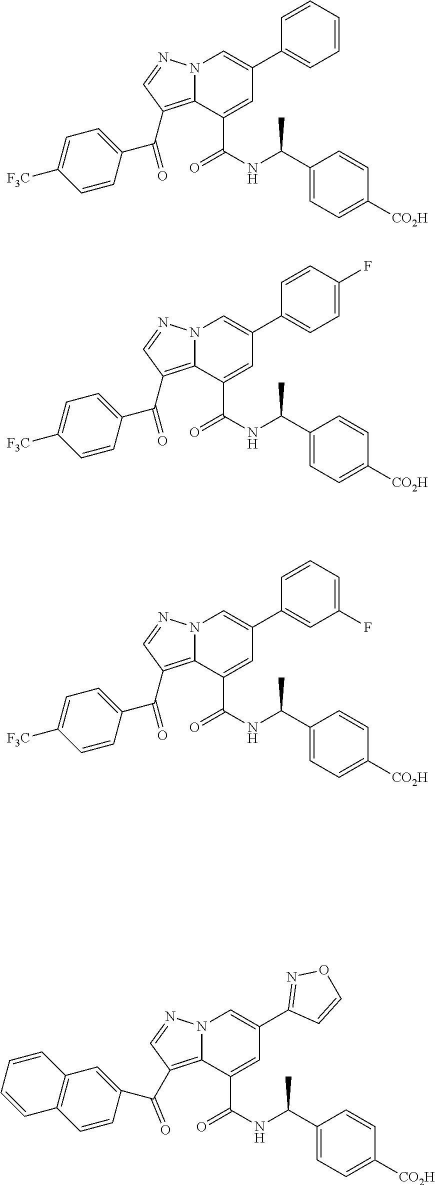 [SMILES: C[C@H](NC(=O)c1cc(-c2ccccc2)cn2ncc(C(=O)c3ccc(C(F)(F)F)cc3)c12)c1ccc(C(=O)O)cc1 ; C[C@H](NC(=O)c1cc(-c2ccc(F)cc2)cn2ncc(C(=O)c3ccc(C(F)(F)F)cc3)c12)c1ccc(C(=O)O)cc1 ; C[C@H](NC(=O)c1cc(-c2cccc(F)c2)cn2ncc(C(=O)c3ccc(C(F)(F)F)cc3)c12)c1ccc(C(=O)O)cc1 ; C[C@H](NC(=O)c1cc(-c2ccon2)cn2ncc(C(=O)c3ccc4ccccc4c3)c12)c1ccc(C(=O)O)cc1]

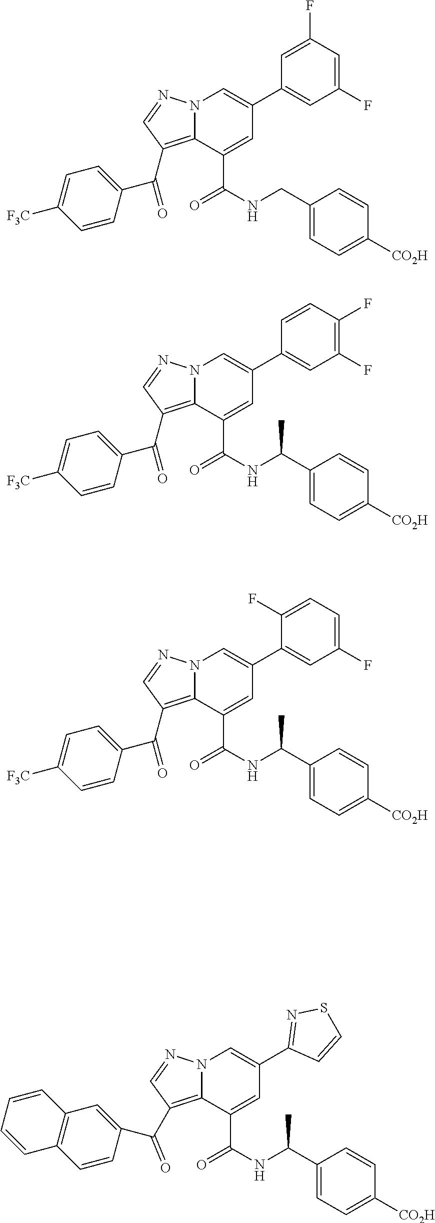 [SMILES: O=C(O)c1ccc(CNC(=O)c2cc(-c3cc(F)cc(F)c3)cn3ncc(C(=O)c4ccc(C(F)(F)F)cc4)c23)cc1 ; C[C@H](NC(=O)c1cc(-c2ccc(F)c(F)c2)cn2ncc(C(=O)c3ccc(C(F)(F)F)cc3)c12)c1ccc(C(=O)O)cc1 ; C[C@H](NC(=O)c1cc(-c2cc(F)ccc2F)cn2ncc(C(=O)c3ccc(C(F)(F)F)cc3)c12)c1ccc(C(=O)O)cc1 ; C[C@H](NC(=O)c1cc(-c2ccsn2)cn2ncc(C(=O)c3ccc4ccccc4c3)c12)c1ccc(C(=O)O)cc1]

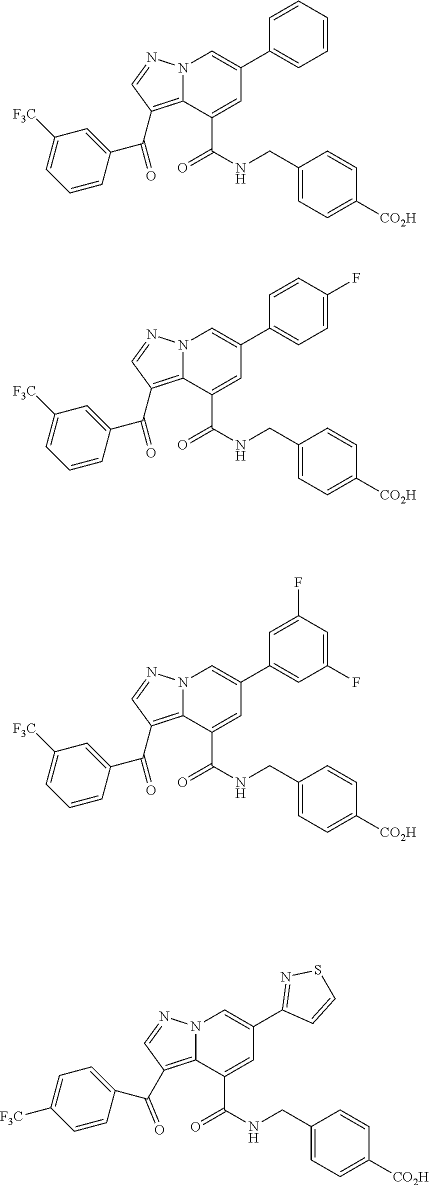 [SMILES: O=C(O)c1ccc(CNC(=O)c2cc(-c3ccccc3)cn3ncc(C(=O)c4cccc(C(F)(F)F)c4)c23)cc1 ; O=C(O)c1ccc(CNC(=O)c2cc(-c3ccc(F)cc3)cn3ncc(C(=O)c4cccc(C(F)(F)F)c4)c23)cc1 ; O=C(O)c1ccc(CNC(=O)c2cc(-c3cc(F)cc(F)c3)cn3ncc(C(=O)c4cccc(C(F)(F)F)c4)c23)cc1 ; O=C(O)c1ccc(CNC(=O)c2cc(-c3ccsn3)cn3ncc(C(=O)c4ccc(C(F)(F)F)cc4)c23)cc1]

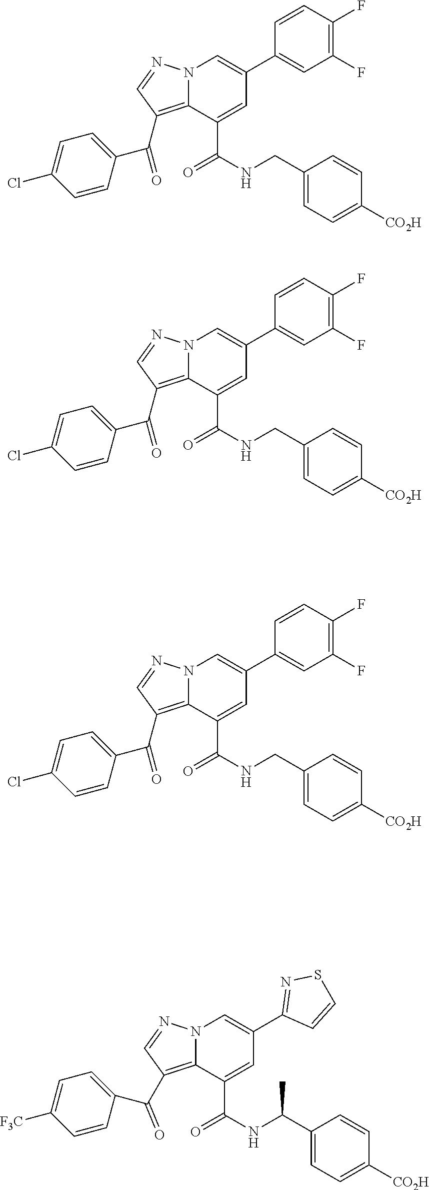 [SMILES: O=C(O)c1ccc(CNC(=O)c2cc(-c3ccc(F)c(F)c3)cn3ncc(C(=O)c4ccc(Cl)cc4)c23)cc1 ; O=C(O)c1ccc(CNC(=O)c2cc(-c3ccc(F)c(F)c3)cn3ncc(C(=O)c4ccc(Cl)cc4)c23)cc1 ; O=C(O)c1ccc(CNC(=O)c2cc(-c3ccc(F)c(F)c3)cn3ncc(C(=O)c4ccc(Cl)cc4)c23)cc1 ; C[C@H](NC(=O)c1cc(-c2ccsn2)cn2ncc(C(=O)c3ccc(C(F)(F)F)cc3)c12)c1ccc(C(=O)O)cc1]

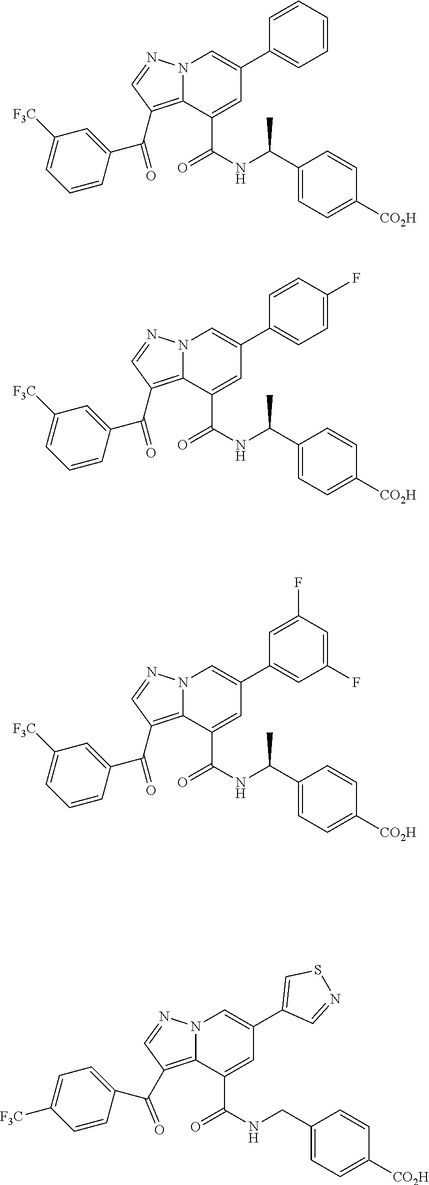 [SMILES: C[C@H](NC(=O)c1cc(-c2ccccc2)cn2ncc(C(=O)c3cccc(C(F)(F)F)c3)c12)c1ccc(C(=O)O)cc1 ; C[C@H](NC(=O)c1cc(-c2ccc(F)cc2)cn2ncc(C(=O)c3cccc(C(F)(F)F)c3)c12)c1ccc(C(=O)O)cc1 ; C[C@H](NC(=O)c1cc(-c2cc(F)cc(F)c2)cn2ncc(C(=O)c3cccc(C(F)(F)F)c3)c12)c1ccc(C(=O)O)cc1 ; O=C(O)c1ccc(CNC(=O)c2cc(-c3cnsc3)cn3ncc(C(=O)c4ccc(C(F)(F)F)cc4)c23)cc1]

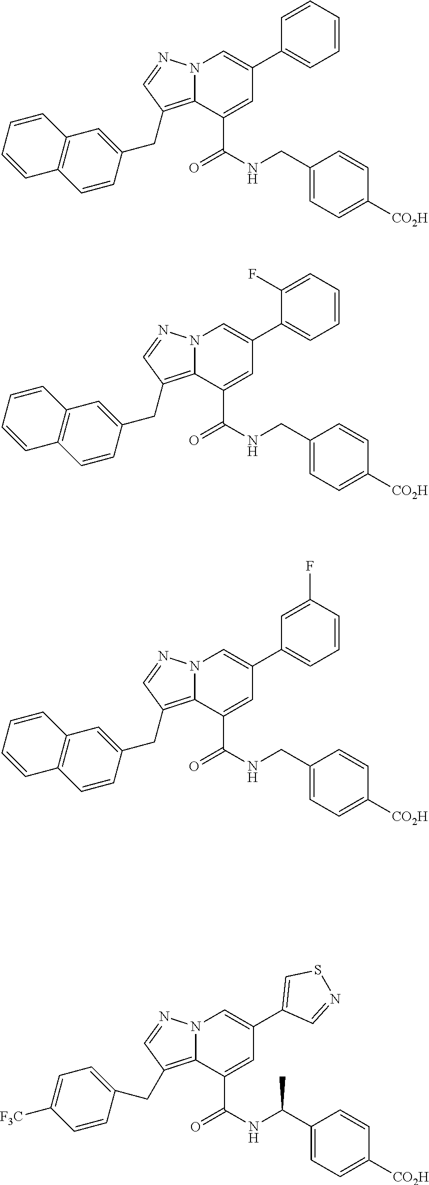 [SMILES: O=C(O)c1ccc(CNC(=O)c2cc(-c3ccccc3)cn3ncc(Cc4ccc5ccccc5c4)c23)cc1 ; O=C(O)c1ccc(CNC(=O)c2cc(-c3ccccc3F)cn3ncc(Cc4ccc5ccccc5c4)c23)cc1 ; O=C(O)c1ccc(CNC(=O)c2cc(-c3cccc(F)c3)cn3ncc(Cc4ccc5ccccc5c4)c23)cc1 ; C[C@H](NC(=O)c1cc(-c2cnsc2)cn2ncc(Cc3ccc(C(F)(F)F)cc3)c12)c1ccc(C(=O)O)cc1]

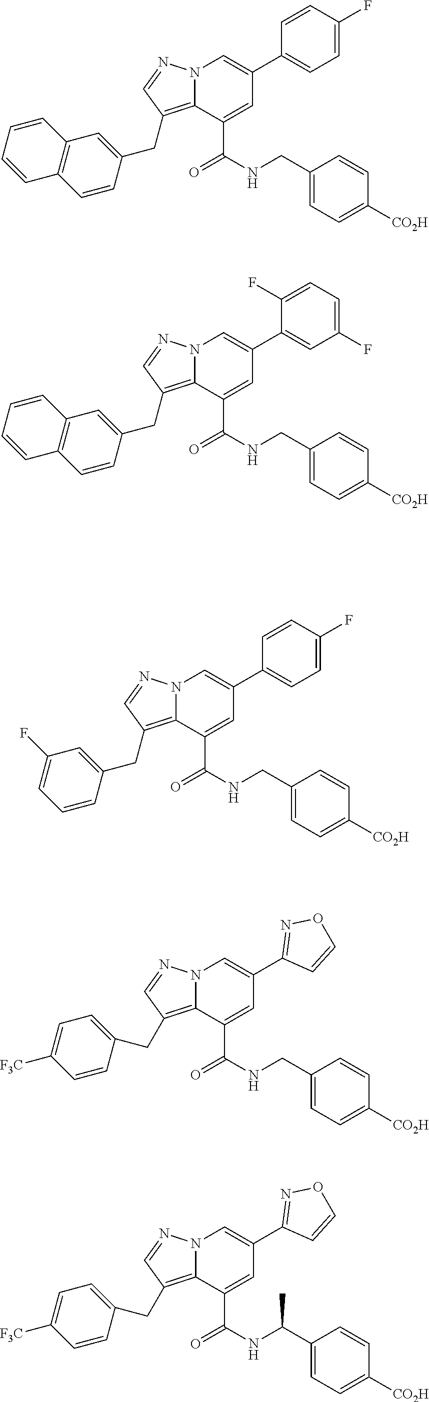 [SMILES: O=C(O)c1ccc(CNC(=O)c2cc(-c3ccc(F)cc3)cn3ncc(Cc4ccc5ccccc5c4)c23)cc1 ; O=C(O)c1ccc(CNC(=O)c2cc(-c3cc(F)ccc3F)cn3ncc(Cc4ccc5ccccc5c4)c23)cc1 ; O=C(O)c1ccc(CNC(=O)c2cc(-c3ccc(F)cc3)cn3ncc(Cc4cccc(F)c4)c23)cc1 ; O=C(O)c1ccc(CNC(=O)c2cc(-c3ccon3)cn3ncc(Cc4ccc(C(F)(F)F)cc4)c23)cc1 ; C[C@H](NC(=O)c1cc(-c2ccon2)cn2ncc(Cc3ccc(C(F)(F)F)cc3)c12)c1ccc(C(=O)O)cc1]

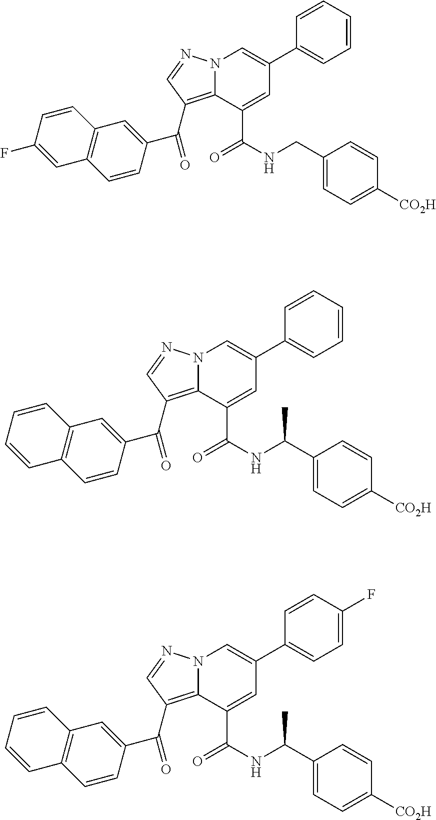 [SMILES: O=C(O)c1ccc(CNC(=O)c2cc(-c3ccccc3)cn3ncc(C(=O)c4ccc5cc(F)ccc5c4)c23)cc1 ; C[C@H](NC(=O)c1cc(-c2ccccc2)cn2ncc(C(=O)c3ccc4ccccc4c3)c12)c1ccc(C(=O)O)cc1 ; C[C@H](NC(=O)c1cc(-c2ccc(F)cc2)cn2ncc(C(=O)c3ccc4ccccc4c3)c12)c1ccc(C(=O)O)cc1]

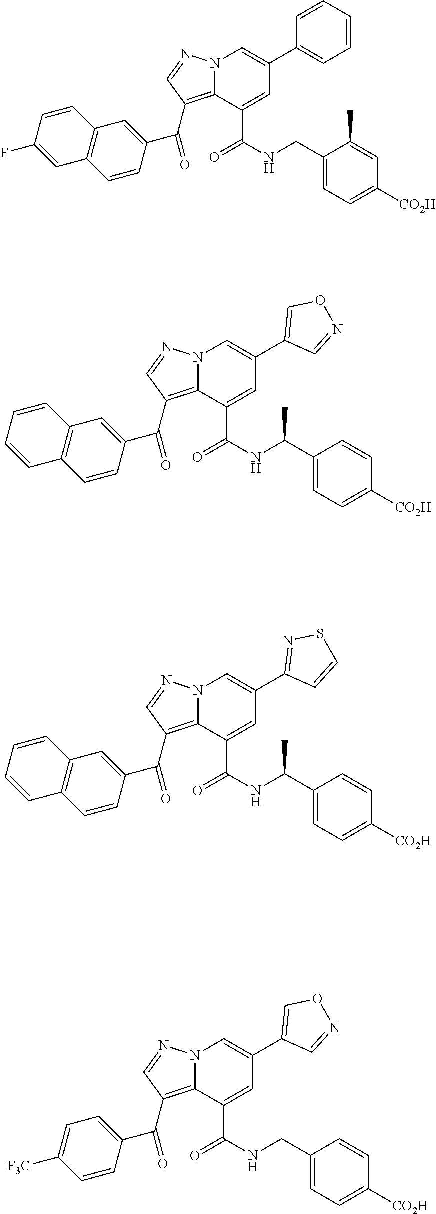

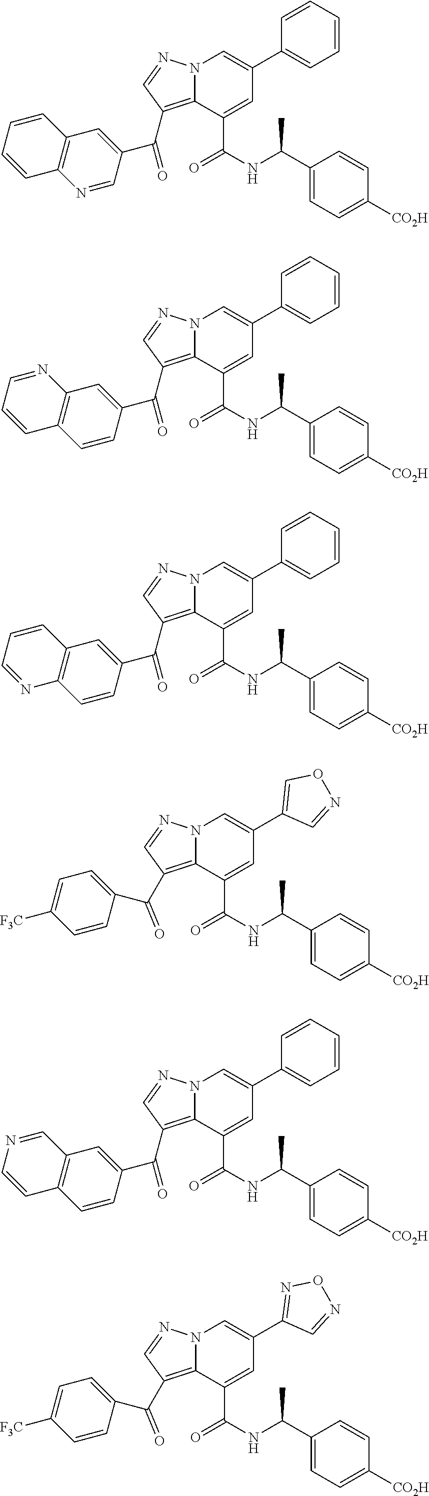 [SMILES: C[C@H](NC(=O)c1cc(-c2ccccc2)cn2ncc(C(=O)c3cnc4ccccc4c3)c12)c1ccc(C(=O)O)cc1 ; C[C@H](NC(=O)c1cc(-c2ccccc2)cn2ncc(C(=O)c3ccc4cccnc4c3)c12)c1ccc(C(=O)O)cc1 ; C[C@H](NC(=O)c1cc(-c2ccccc2)cn2ncc(C(=O)c3ccc4ncccc4c3)c12)c1ccc(C(=O)O)cc1 ; C[C@H](NC(=O)c1cc(-c2cnoc2)cn2ncc(C(=O)c3ccc(C(F)(F)F)cc3)c12)c1ccc(C(=O)O)cc1 ; C[C@H](NC(=O)c1cc(-c2ccccc2)cn2ncc(C(=O)c3ccc4ccncc4c3)c12)c1ccc(C(=O)O)cc1 ; C[C@H](NC(=O)c1cc(-c2cnon2)cn2ncc(C(=O)c3ccc(C(F)(F)F)cc3)c12)c1ccc(C(=O)O)cc1]

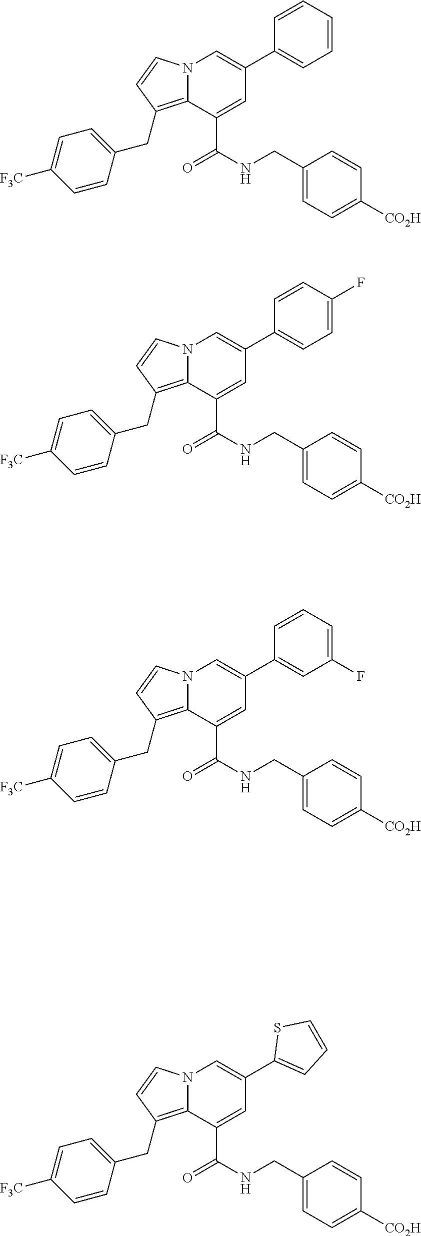 [SMILES: O=C(O)c1ccc(CNC(=O)c2cc(-c3ccccc3)cn3ccc(Cc4ccc(C(F)(F)F)cc4)c23)cc1 ; O=C(O)c1ccc(CNC(=O)c2cc(-c3ccc(F)cc3)cn3ccc(Cc4ccc(C(F)(F)F)cc4)c23)cc1 ; O=C(O)c1ccc(CNC(=O)c2cc(-c3cccc(F)c3)cn3ccc(Cc4ccc(C(F)(F)F)cc4)c23)cc1 ; O=C(O)c1ccc(CNC(=O)c2cc(-c3cccs3)cn3ccc(Cc4ccc(C(F)(F)F)cc4)c23)cc1]

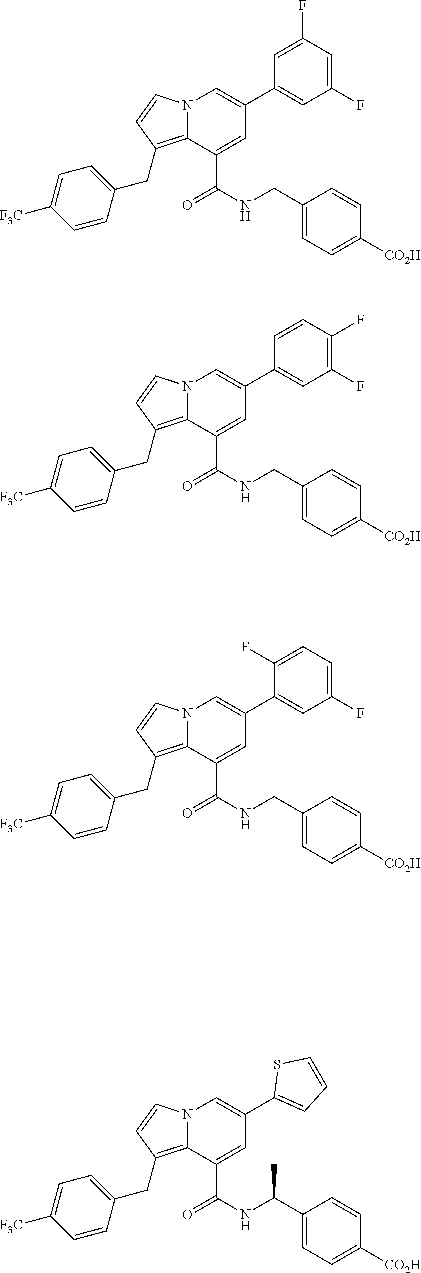 [SMILES: O=C(O)c1ccc(CNC(=O)c2cc(-c3cc(F)cc(F)c3)cn3ccc(Cc4ccc(C(F)(F)F)cc4)c23)cc1 ; O=C(O)c1ccc(CNC(=O)c2cc(-c3ccc(F)c(F)c3)cn3ccc(Cc4ccc(C(F)(F)F)cc4)c23)cc1 ; O=C(O)c1ccc(CNC(=O)c2cc(-c3cc(F)ccc3F)cn3ccc(Cc4ccc(C(F)(F)F)cc4)c23)cc1 ; C[C@H](NC(=O)c1cc(-c2cccs2)cn2ccc(Cc3ccc(C(F)(F)F)cc3)c12)c1ccc(C(=O)O)cc1]

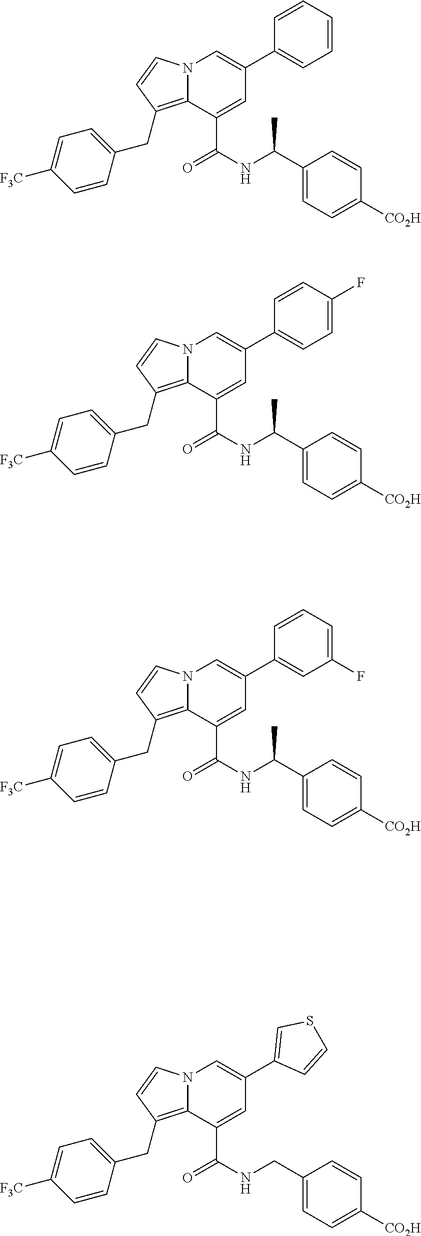 [SMILES: C[C@H](NC(=O)c1cc(-c2ccccc2)cn2ccc(Cc3ccc(C(F)(F)F)cc3)c12)c1ccc(C(=O)O)cc1 ; C[C@H](NC(=O)c1cc(-c2ccc(F)cc2)cn2ccc(Cc3ccc(C(F)(F)F)cc3)c12)c1ccc(C(=O)O)cc1 ; C[C@H](NC(=O)c1cc(-c2cccc(F)c2)cn2ccc(Cc3ccc(C(F)(F)F)cc3)c12)c1ccc(C(=O)O)cc1 ; O=C(O)c1ccc(CNC(=O)c2cc(-c3ccsc3)cn3ccc(Cc4ccc(C(F)(F)F)cc4)c23)cc1]

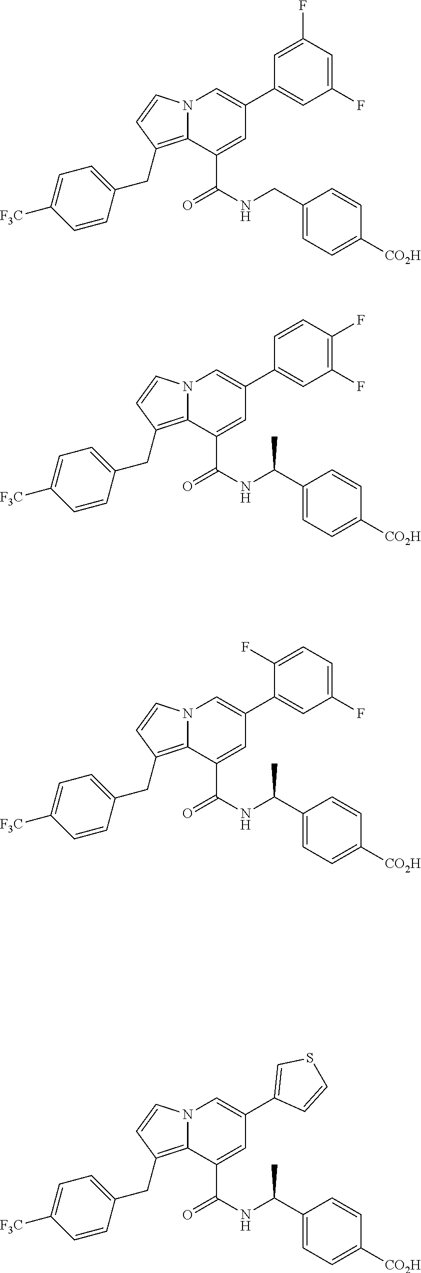 [SMILES: O=C(O)c1ccc(CNC(=O)c2cc(-c3cc(F)cc(F)c3)cn3ccc(Cc4ccc(C(F)(F)F)cc4)c23)cc1 ; C[C@H](NC(=O)c1cc(-c2ccc(F)c(F)c2)cn2ccc(Cc3ccc(C(F)(F)F)cc3)c12)c1ccc(C(=O)O)cc1 ; C[C@H](NC(=O)c1cc(-c2cc(F)ccc2F)cn2ccc(Cc3ccc(C(F)(F)F)cc3)c12)c1ccc(C(=O)O)cc1 ; C[C@H](NC(=O)c1cc(-c2ccsc2)cn2ccc(Cc3ccc(C(F)(F)F)cc3)c12)c1ccc(C(=O)O)cc1]

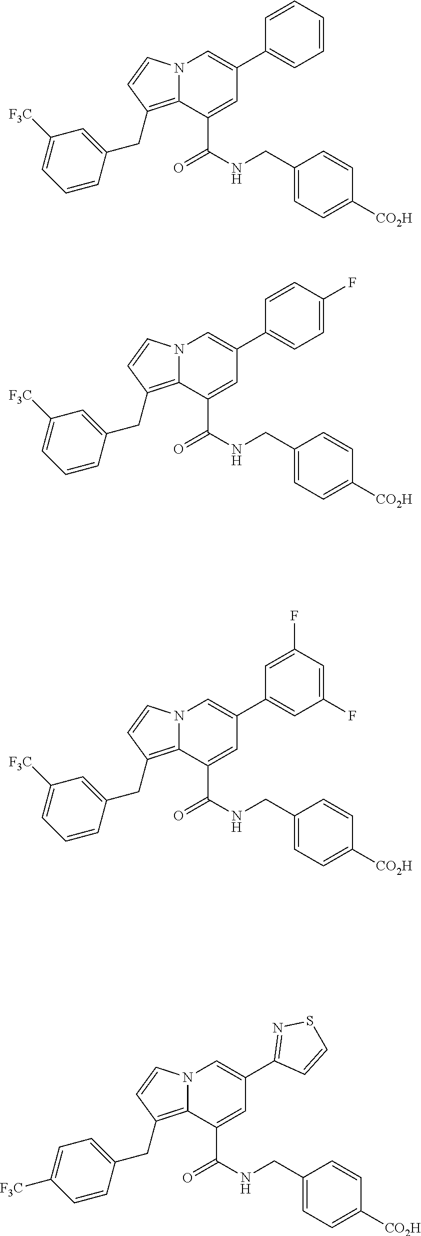 [SMILES: O=C(O)c1ccc(CNC(=O)c2cc(-c3ccccc3)cn3ccc(Cc4cccc(C(F)(F)F)c4)c23)cc1 ; O=C(O)c1ccc(CNC(=O)c2cc(-c3ccc(F)cc3)cn3ccc(Cc4cccc(C(F)(F)F)c4)c23)cc1 ; O=C(O)c1ccc(CNC(=O)c2cc(-c3cc(F)cc(F)c3)cn3ccc(Cc4cccc(C(F)(F)F)c4)c23)cc1 ; O=C(O)c1ccc(CNC(=O)c2cc(-c3ccsn3)cn3ccc(Cc4ccc(C(F)(F)F)cc4)c23)cc1]

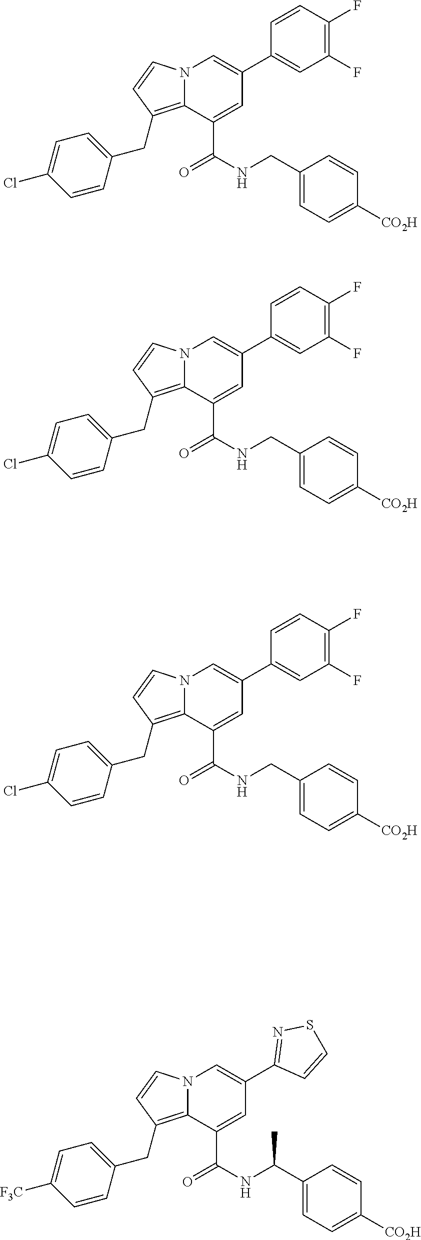 [SMILES: O=C(O)c1ccc(CNC(=O)c2cc(-c3ccc(F)c(F)c3)cn3ccc(Cc4ccc(Cl)cc4)c23)cc1 ; O=C(O)c1ccc(CNC(=O)c2cc(-c3ccc(F)c(F)c3)cn3ccc(Cc4ccc(Cl)cc4)c23)cc1 ; O=C(O)c1ccc(CNC(=O)c2cc(-c3ccc(F)c(F)c3)cn3ccc(Cc4ccc(Cl)cc4)c23)cc1 ; C[C@H](NC(=O)c1cc(-c2ccsn2)cn2ccc(Cc3ccc(C(F)(F)F)cc3)c12)c1ccc(C(=O)O)cc1]

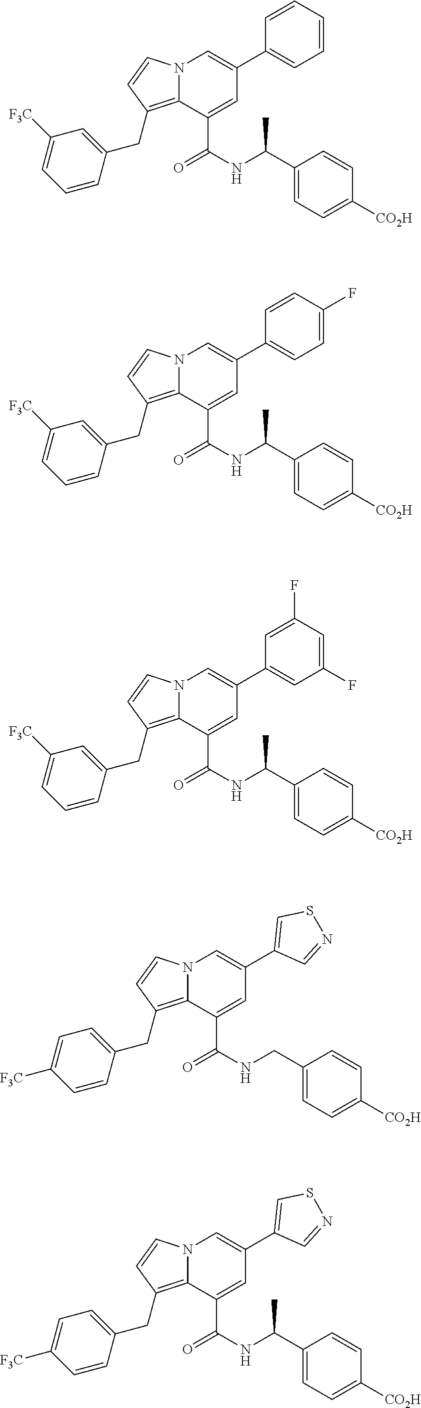 [SMILES: C[C@H](NC(=O)c1cc(-c2ccccc2)cn2ccc(Cc3cccc(C(F)(F)F)c3)c12)c1ccc(C(=O)O)cc1 ; C[C@H](NC(=O)c1cc(-c2ccc(F)cc2)cn2ccc(Cc3cccc(C(F)(F)F)c3)c12)c1ccc(C(=O)O)cc1 ; C[C@H](NC(=O)c1cc(-c2cc(F)cc(F)c2)cn2ccc(Cc3cccc(C(F)(F)F)c3)c12)c1ccc(C(=O)O)cc1 ; O=C(O)c1ccc(CNC(=O)c2cc(-c3cnsc3)cn3ccc(Cc4ccc(C(F)(F)F)cc4)c23)cc1 ; C[C@H](NC(=O)c1cc(-c2cnsc2)cn2ccc(Cc3ccc(C(F)(F)F)cc3)c12)c1ccc(C(=O)O)cc1]

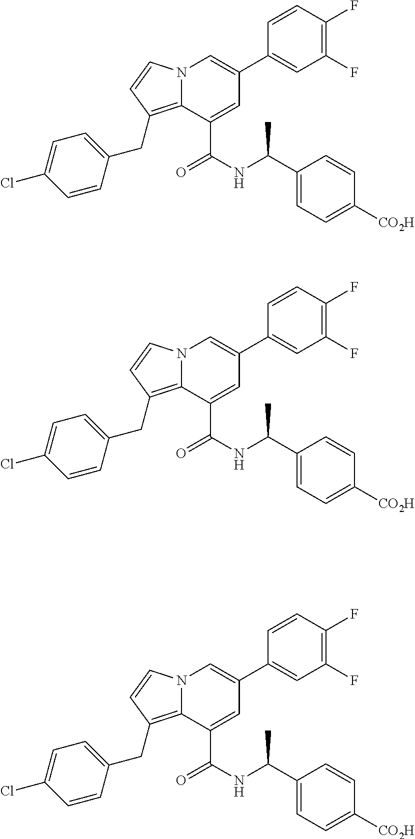 [SMILES: C[C@H](NC(=O)c1cc(-c2ccc(F)c(F)c2)cn2ccc(Cc3ccc(Cl)cc3)c12)c1ccc(C(=O)O)cc1 ; C[C@H](NC(=O)c1cc(-c2ccc(F)c(F)c2)cn2ccc(Cc3ccc(Cl)cc3)c12)c1ccc(C(=O)O)cc1 ; C[C@H](NC(=O)c1cc(-c2ccc(F)c(F)c2)cn2ccc(Cc3ccc(Cl)cc3)c12)c1ccc(C(=O)O)cc1]

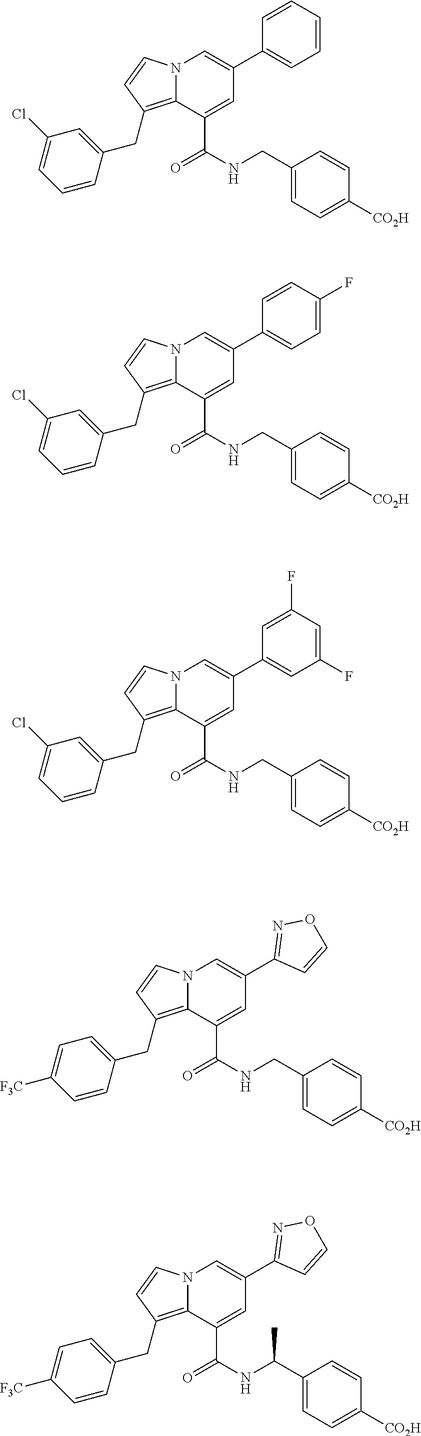 [SMILES: O=C(O)c1ccc(CNC(=O)c2cc(-c3ccccc3)cn3ccc(Cc4cccc(Cl)c4)c23)cc1 ; O=C(O)c1ccc(CNC(=O)c2cc(-c3ccc(F)cc3)cn3ccc(Cc4cccc(Cl)c4)c23)cc1 ; O=C(O)c1ccc(CNC(=O)c2cc(-c3cc(F)cc(F)c3)cn3ccc(Cc4cccc(Cl)c4)c23)cc1 ; O=C(O)c1ccc(CNC(=O)c2cc(-c3ccon3)cn3ccc(Cc4ccc(C(F)(F)F)cc4)c23)cc1 ; C[C@H](NC(=O)c1cc(-c2ccon2)cn2ccc(Cc3ccc(C(F)(F)F)cc3)c12)c1ccc(C(=O)O)cc1]

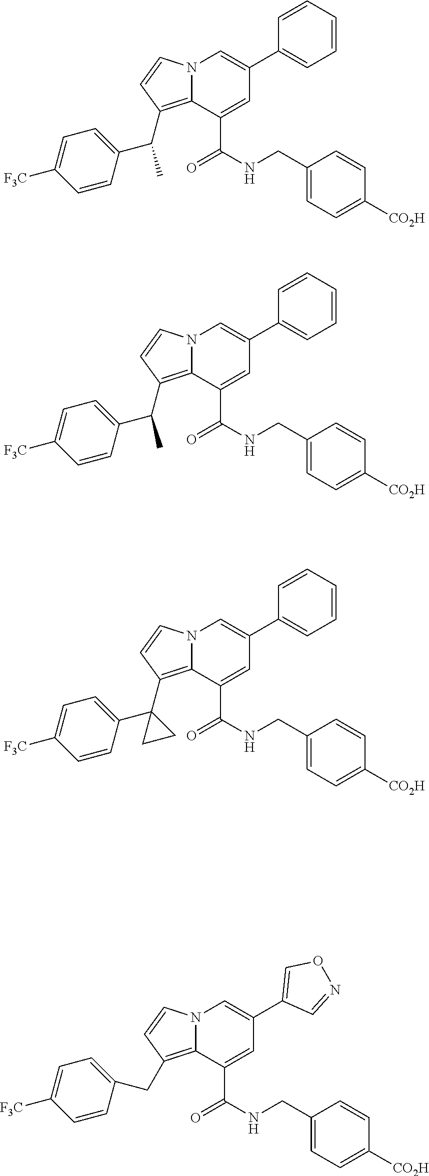 [SMILES: C[C@H](c1ccc(C(F)(F)F)cc1)c1ccn2cc(-c3ccccc3)cc(C(=O)NCc3ccc(C(=O)O)cc3)c12 ; C[C@@H](c1ccc(C(F)(F)F)cc1)c1ccn2cc(-c3ccccc3)cc(C(=O)NCc3ccc(C(=O)O)cc3)c12 ; O=C(O)c1ccc(CNC(=O)c2cc(-c3ccccc3)cn3ccc(C4(c5ccc(C(F)(F)F)cc5)CC4)c23)cc1 ; O=C(O)c1ccc(CNC(=O)c2cc(-c3cnoc3)cn3ccc(Cc4ccc(C(F)(F)F)cc4)c23)cc1]

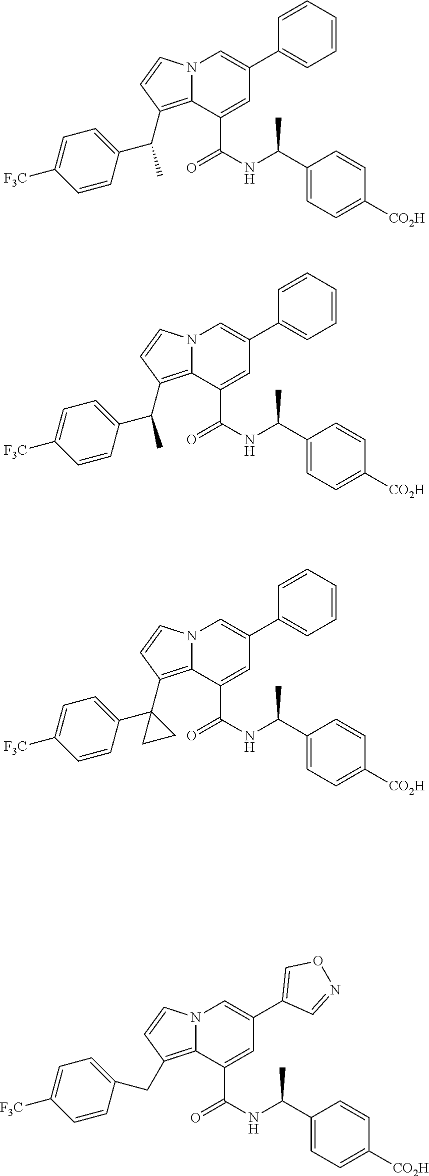 [SMILES: C[C@H](c1ccc(C(F)(F)F)cc1)c1ccn2cc(-c3ccccc3)cc(C(=O)N[C@@H](C)c3ccc(C(=O)O)cc3)c12 ; C[C@@H](c1ccc(C(F)(F)F)cc1)c1ccn2cc(-c3ccccc3)cc(C(=O)N[C@@H](C)c3ccc(C(=O)O)cc3)c12 ; C[C@H](NC(=O)c1cc(-c2ccccc2)cn2ccc(C3(c4ccc(C(F)(F)F)cc4)CC3)c12)c1ccc(C(=O)O)cc1 ; C[C@H](NC(=O)c1cc(-c2cnoc2)cn2ccc(Cc3ccc(C(F)(F)F)cc3)c12)c1ccc(C(=O)O)cc1]

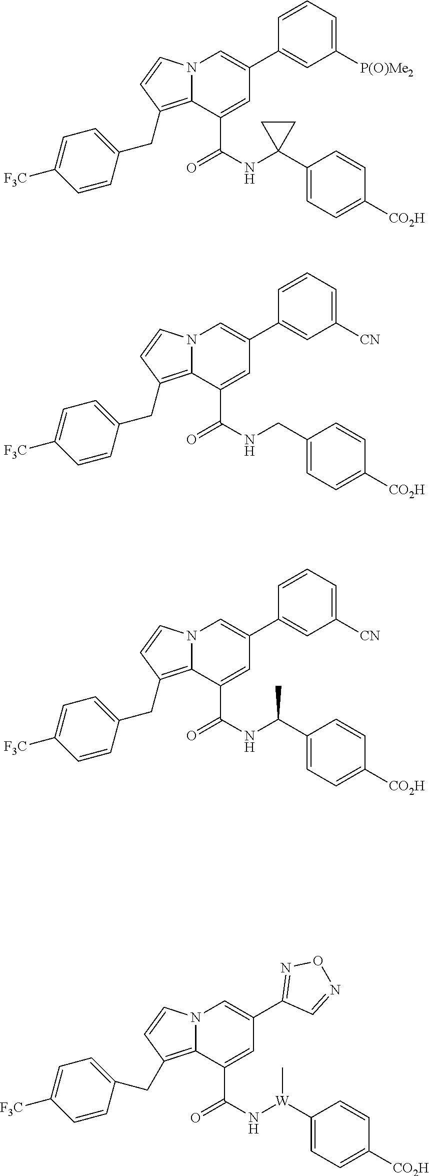

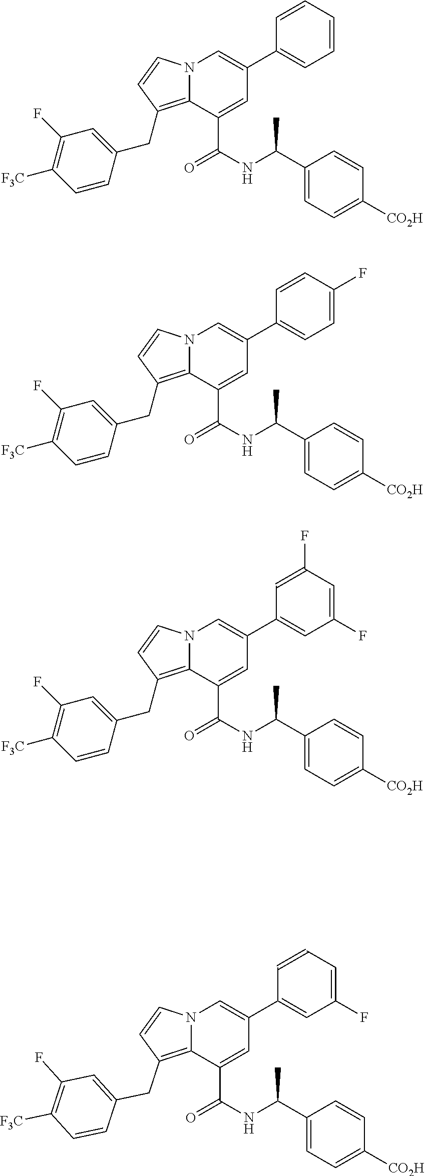 [SMILES: C[C@H](NC(=O)c1cc(-c2ccccc2)cn2ccc(Cc3ccc(C(F)(F)F)c(F)c3)c12)c1ccc(C(=O)O)cc1 ; C[C@H](NC(=O)c1cc(-c2ccc(F)cc2)cn2ccc(Cc3ccc(C(F)(F)F)c(F)c3)c12)c1ccc(C(=O)O)cc1 ; C[C@H](NC(=O)c1cc(-c2cc(F)cc(F)c2)cn2ccc(Cc3ccc(C(F)(F)F)c(F)c3)c12)c1ccc(C(=O)O)cc1 ; C[C@H](NC(=O)c1cc(-c2cccc(F)c2)cn2ccc(Cc3ccc(C(F)(F)F)c(F)c3)c12)c1ccc(C(=O)O)cc1]

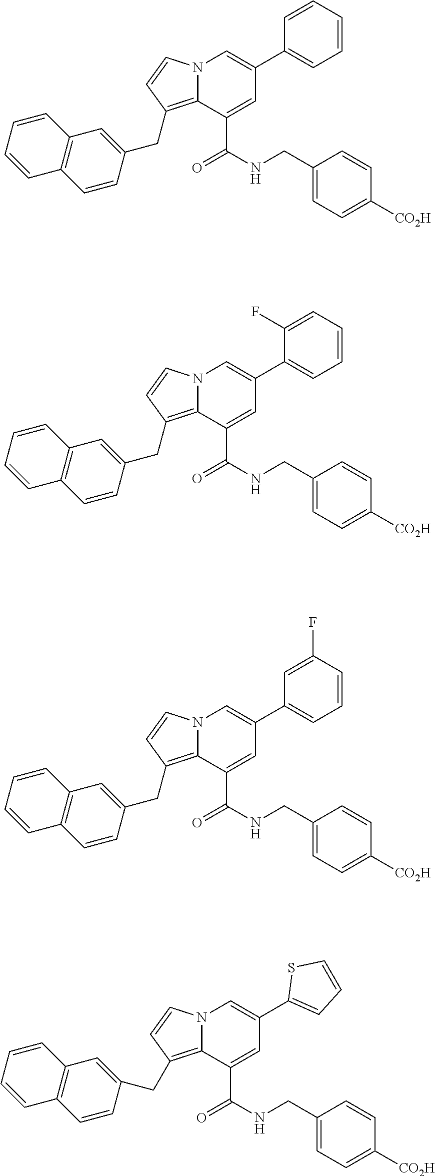 [SMILES: O=C(O)c1ccc(CNC(=O)c2cc(-c3ccccc3)cn3ccc(Cc4ccc5ccccc5c4)c23)cc1 ; O=C(O)c1ccc(CNC(=O)c2cc(-c3ccccc3F)cn3ccc(Cc4ccc5ccccc5c4)c23)cc1 ; O=C(O)c1ccc(CNC(=O)c2cc(-c3cccc(F)c3)cn3ccc(Cc4ccc5ccccc5c4)c23)cc1 ; O=C(O)c1ccc(CNC(=O)c2cc(-c3cccs3)cn3ccc(Cc4ccc5ccccc5c4)c23)cc1]

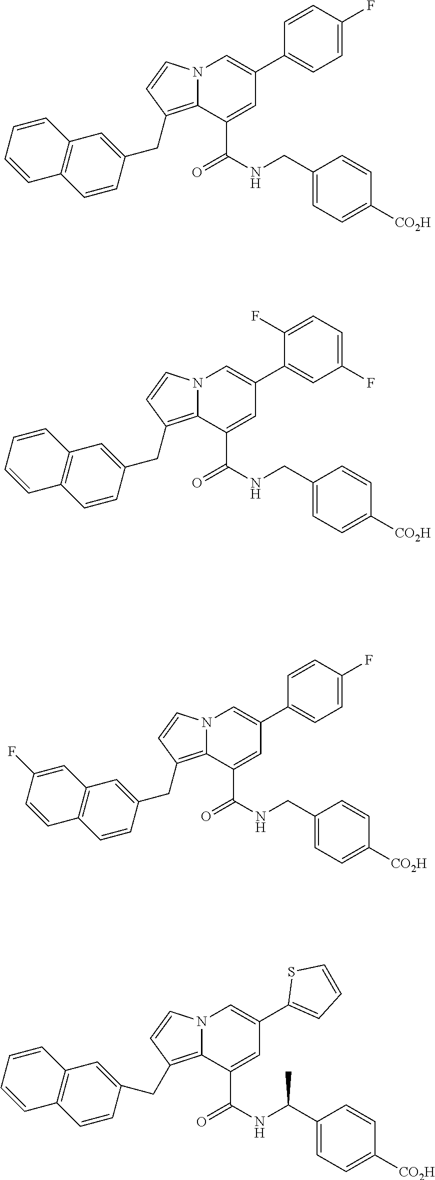 [SMILES: O=C(O)c1ccc(CNC(=O)c2cc(-c3ccc(F)cc3)cn3ccc(Cc4ccc5ccccc5c4)c23)cc1 ; O=C(O)c1ccc(CNC(=O)c2cc(-c3cc(F)ccc3F)cn3ccc(Cc4ccc5ccccc5c4)c23)cc1 ; O=C(O)c1ccc(CNC(=O)c2cc(-c3ccc(F)cc3)cn3ccc(Cc4ccc5ccc(F)cc5c4)c23)cc1 ; C[C@H](NC(=O)c1cc(-c2cccs2)cn2ccc(Cc3ccc4ccccc4c3)c12)c1ccc(C(=O)O)cc1]

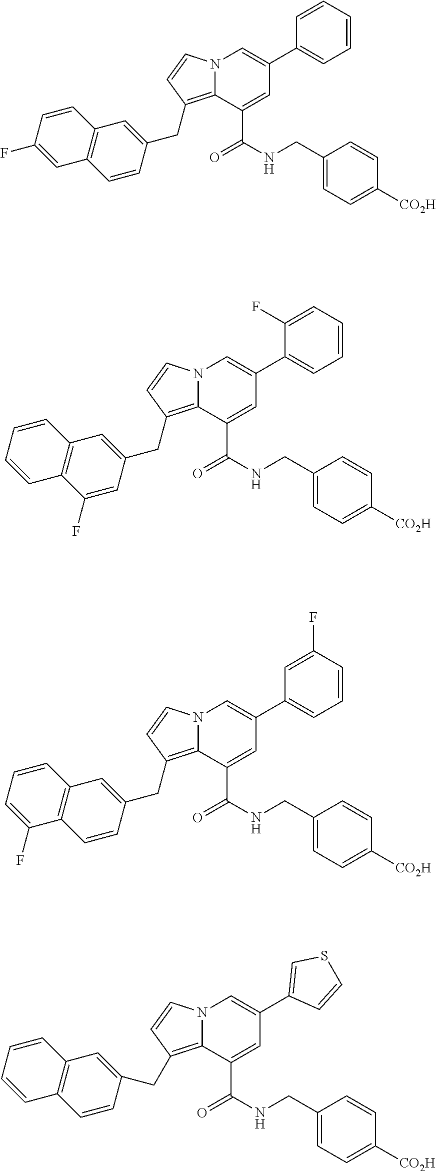 [SMILES: O=C(O)c1ccc(CNC(=O)c2cc(-c3ccccc3)cn3ccc(Cc4ccc5cc(F)ccc5c4)c23)cc1 ; O=C(O)c1ccc(CNC(=O)c2cc(-c3ccccc3F)cn3ccc(Cc4cc(F)c5ccccc5c4)c23)cc1 ; O=C(O)c1ccc(CNC(=O)c2cc(-c3cccc(F)c3)cn3ccc(Cc4ccc5c(F)cccc5c4)c23)cc1 ; O=C(O)c1ccc(CNC(=O)c2cc(-c3ccsc3)cn3ccc(Cc4ccc5ccccc5c4)c23)cc1]

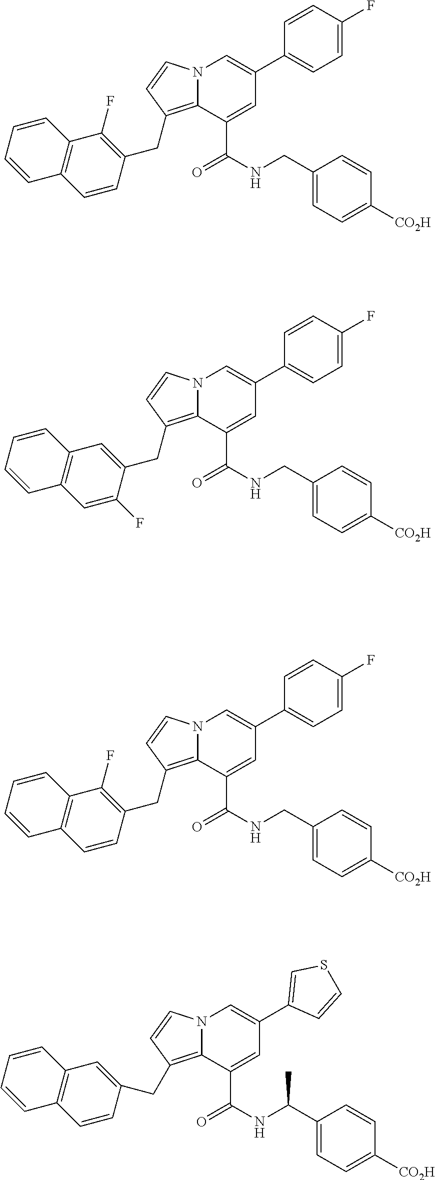 [SMILES: O=C(O)c1ccc(CNC(=O)c2cc(-c3ccc(F)cc3)cn3ccc(Cc4ccc5ccccc5c4F)c23)cc1 ; O=C(O)c1ccc(CNC(=O)c2cc(-c3ccc(F)cc3)cn3ccc(Cc4cc5ccccc5cc4F)c23)cc1 ; O=C(O)c1ccc(CNC(=O)c2cc(-c3ccc(F)cc3)cn3ccc(Cc4ccc5ccccc5c4F)c23)cc1 ; C[C@H](NC(=O)c1cc(-c2ccsc2)cn2ccc(Cc3ccc4ccccc4c3)c12)c1ccc(C(=O)O)cc1]

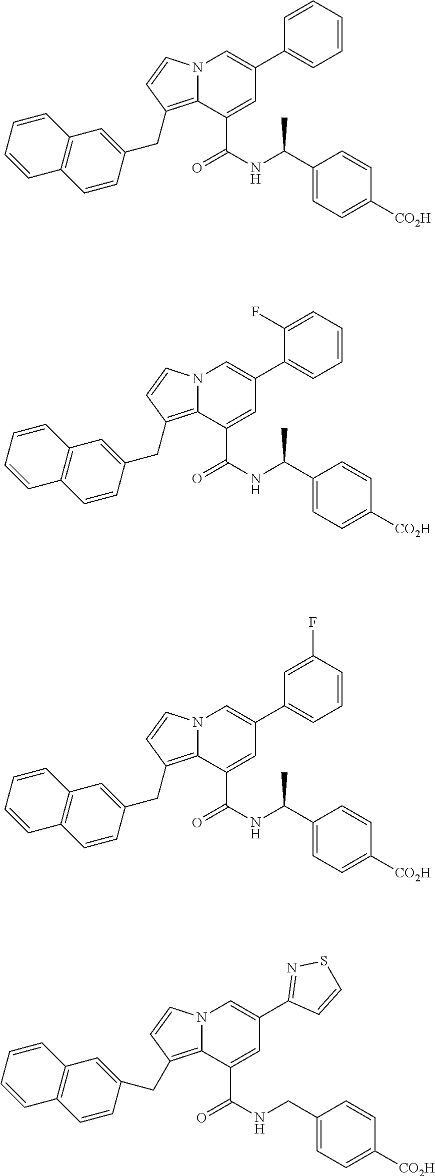 [SMILES: C[C@H](NC(=O)c1cc(-c2ccccc2)cn2ccc(Cc3ccc4ccccc4c3)c12)c1ccc(C(=O)O)cc1 ; C[C@H](NC(=O)c1cc(-c2ccccc2F)cn2ccc(Cc3ccc4ccccc4c3)c12)c1ccc(C(=O)O)cc1 ; C[C@H](NC(=O)c1cc(-c2cccc(F)c2)cn2ccc(Cc3ccc4ccccc4c3)c12)c1ccc(C(=O)O)cc1 ; O=C(O)c1ccc(CNC(=O)c2cc(-c3ccsn3)cn3ccc(Cc4ccc5ccccc5c4)c23)cc1]

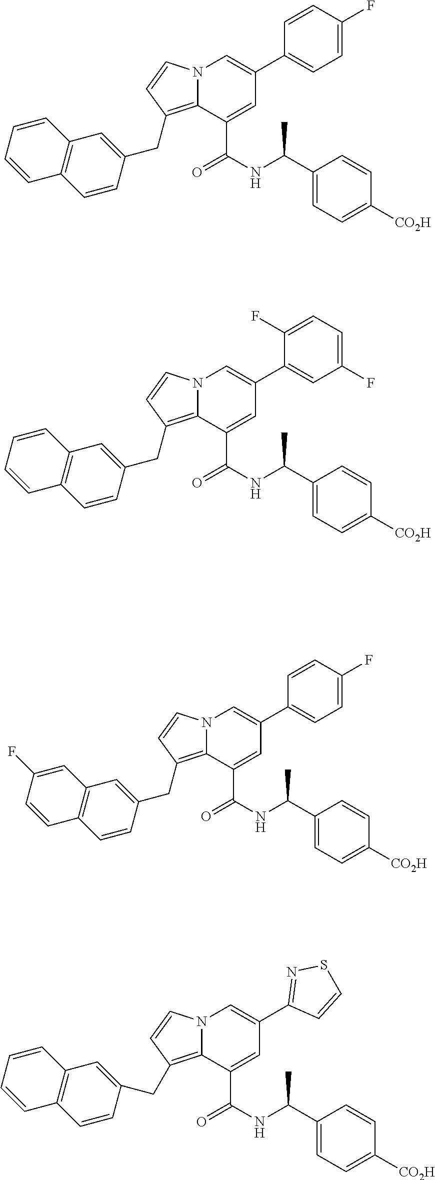 [SMILES: C[C@H](NC(=O)c1cc(-c2ccc(F)cc2)cn2ccc(Cc3ccc4ccccc4c3)c12)c1ccc(C(=O)O)cc1 ; C[C@H](NC(=O)c1cc(-c2cc(F)ccc2F)cn2ccc(Cc3ccc4ccccc4c3)c12)c1ccc(C(=O)O)cc1 ; C[C@H](NC(=O)c1cc(-c2ccc(F)cc2)cn2ccc(Cc3ccc4ccc(F)cc4c3)c12)c1ccc(C(=O)O)cc1 ; C[C@H](NC(=O)c1cc(-c2ccsn2)cn2ccc(Cc3ccc4ccccc4c3)c12)c1ccc(C(=O)O)cc1]

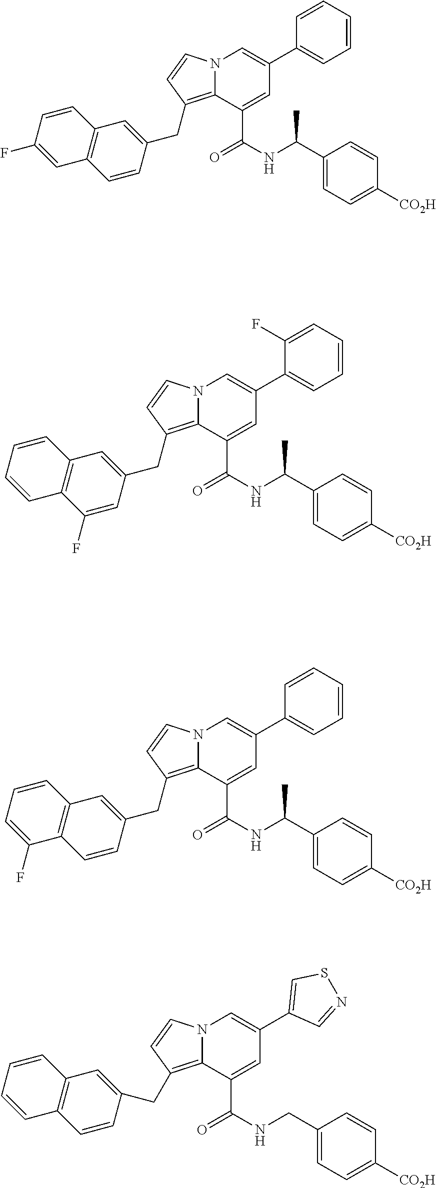 [SMILES: C[C@H](NC(=O)c1cc(-c2ccccc2)cn2ccc(Cc3ccc4cc(F)ccc4c3)c12)c1ccc(C(=O)O)cc1 ; C[C@H](NC(=O)c1cc(-c2ccccc2F)cn2ccc(Cc3cc(F)c4ccccc4c3)c12)c1ccc(C(=O)O)cc1 ; C[C@H](NC(=O)c1cc(-c2ccccc2)cn2ccc(Cc3ccc4c(F)cccc4c3)c12)c1ccc(C(=O)O)cc1 ; O=C(O)c1ccc(CNC(=O)c2cc(-c3cnsc3)cn3ccc(Cc4ccc5ccccc5c4)c23)cc1]

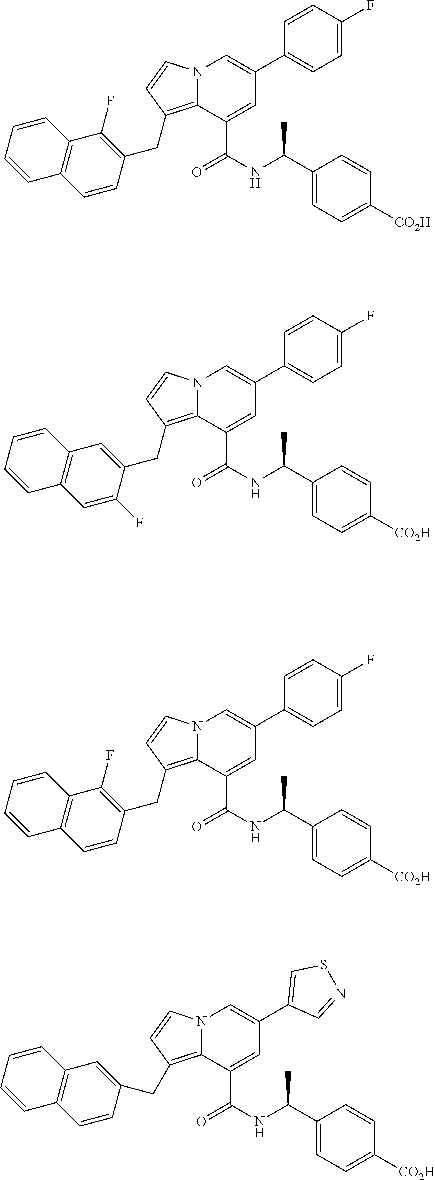 [SMILES: C[C@H](NC(=O)c1cc(-c2ccc(F)cc2)cn2ccc(Cc3ccc4ccccc4c3F)c12)c1ccc(C(=O)O)cc1 ; C[C@H](NC(=O)c1cc(-c2ccc(F)cc2)cn2ccc(Cc3cc4ccccc4cc3F)c12)c1ccc(C(=O)O)cc1 ; C[C@H](NC(=O)c1cc(-c2ccc(F)cc2)cn2ccc(Cc3ccc4ccccc4c3F)c12)c1ccc(C(=O)O)cc1 ; C[C@H](NC(=O)c1cc(-c2cnsc2)cn2ccc(Cc3ccc4ccccc4c3)c12)c1ccc(C(=O)O)cc1]

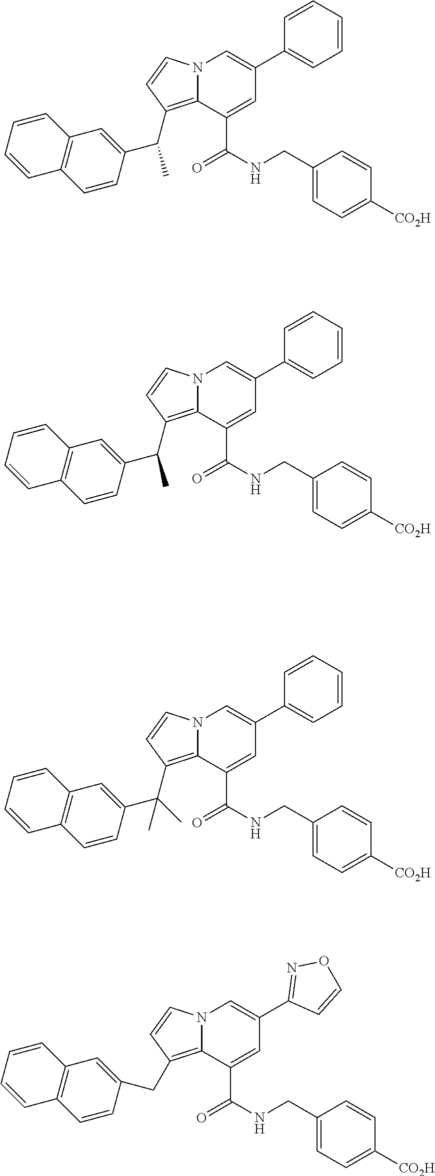 [SMILES: C[C@H](c1ccc2ccccc2c1)c1ccn2cc(-c3ccccc3)cc(C(=O)NCc3ccc(C(=O)O)cc3)c12 ; C[C@@H](c1ccc2ccccc2c1)c1ccn2cc(-c3ccccc3)cc(C(=O)NCc3ccc(C(=O)O)cc3)c12 ; CC(C)(c1ccc2ccccc2c1)c1ccn2cc(-c3ccccc3)cc(C(=O)NCc3ccc(C(=O)O)cc3)c12 ; O=C(O)c1ccc(CNC(=O)c2cc(-c3ccon3)cn3ccc(Cc4ccc5ccccc5c4)c23)cc1]

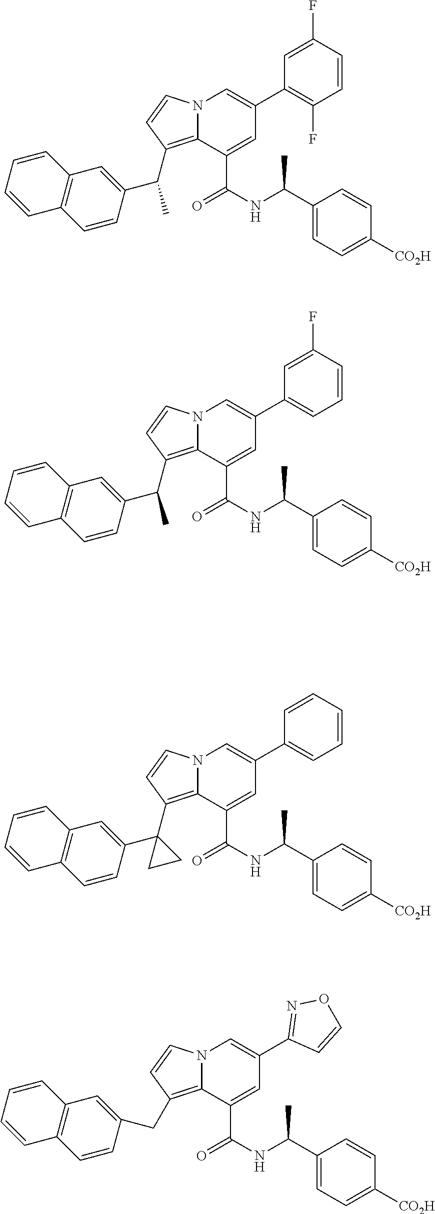 [SMILES: C[C@H](c1ccc2ccccc2c1)c1ccn2cc(-c3cc(F)ccc3F)cc(C(=O)N[C@@H](C)c3ccc(C(=O)O)cc3)c12 ; C[C@@H](c1ccc2ccccc2c1)c1ccn2cc(-c3cccc(F)c3)cc(C(=O)N[C@@H](C)c3ccc(C(=O)O)cc3)c12 ; C[C@H](NC(=O)c1cc(-c2ccccc2)cn2ccc(C3(c4ccc5ccccc5c4)CC3)c12)c1ccc(C(=O)O)cc1 ; C[C@H](NC(=O)c1cc(-c2ccon2)cn2ccc(Cc3ccc4ccccc4c3)c12)c1ccc(C(=O)O)cc1]

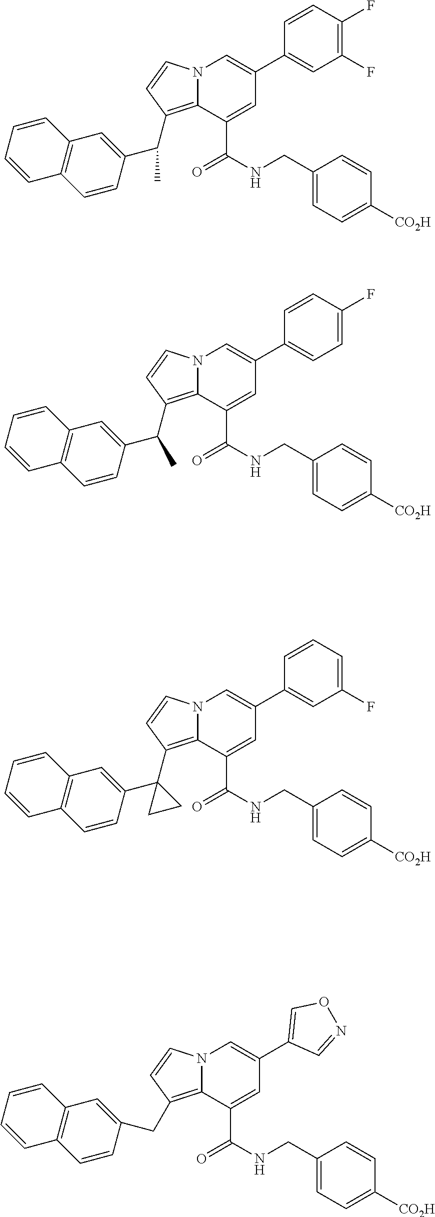 [SMILES: C[C@H](c1ccc2ccccc2c1)c1ccn2cc(-c3ccc(F)c(F)c3)cc(C(=O)NCc3ccc(C(=O)O)cc3)c12 ; C[C@@H](c1ccc2ccccc2c1)c1ccn2cc(-c3ccc(F)cc3)cc(C(=O)NCc3ccc(C(=O)O)cc3)c12 ; O=C(O)c1ccc(CNC(=O)c2cc(-c3cccc(F)c3)cn3ccc(C4(c5ccc6ccccc6c5)CC4)c23)cc1 ; O=C(O)c1ccc(CNC(=O)c2cc(-c3cnoc3)cn3ccc(Cc4ccc5ccccc5c4)c23)cc1]

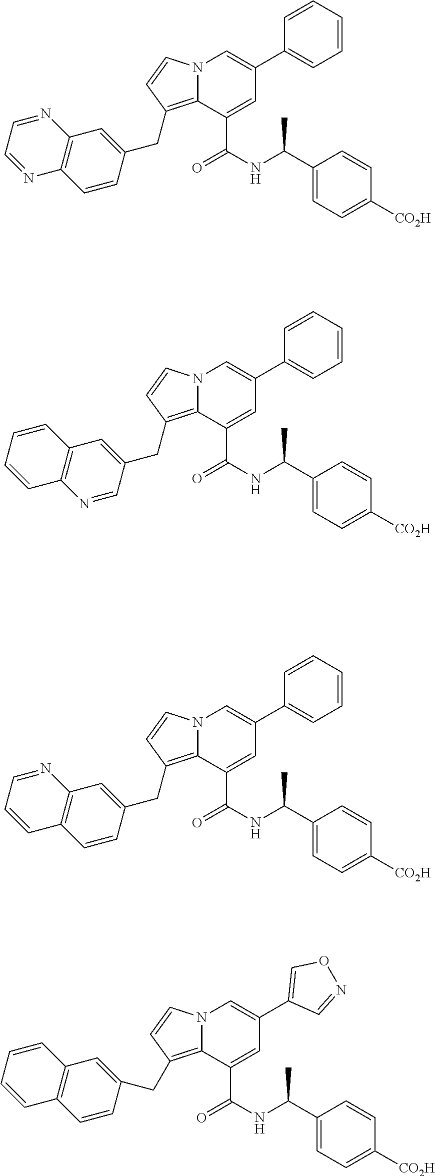 [SMILES: C[C@H](NC(=O)c1cc(-c2ccccc2)cn2ccc(Cc3ccc4nccnc4c3)c12)c1ccc(C(=O)O)cc1 ; C[C@H](NC(=O)c1cc(-c2ccccc2)cn2ccc(Cc3cnc4ccccc4c3)c12)c1ccc(C(=O)O)cc1 ; C[C@H](NC(=O)c1cc(-c2ccccc2)cn2ccc(Cc3ccc4cccnc4c3)c12)c1ccc(C(=O)O)cc1 ; C[C@H](NC(=O)c1cc(-c2cnoc2)cn2ccc(Cc3ccc4ccccc4c3)c12)c1ccc(C(=O)O)cc1]

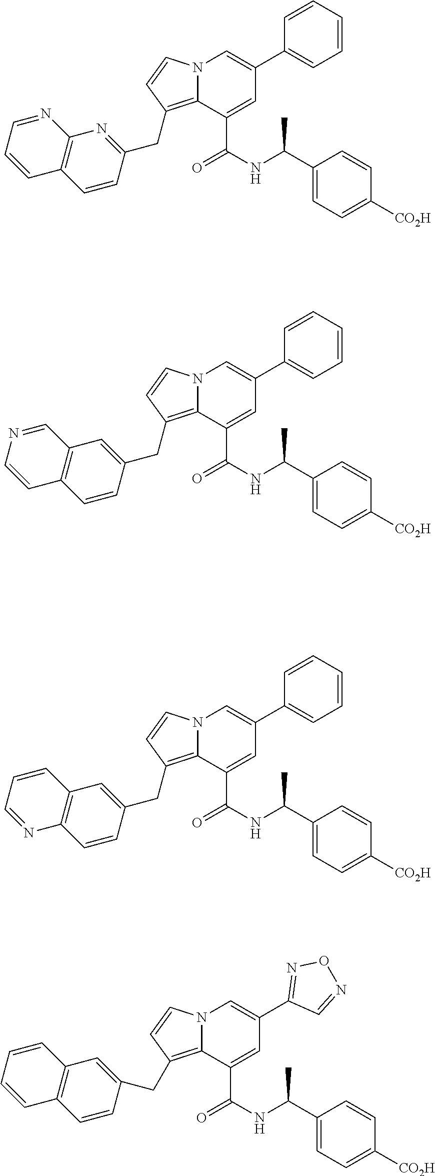 [SMILES: C[C@H](NC(=O)c1cc(-c2ccccc2)cn2ccc(Cc3ccc4cccnc4n3)c12)c1ccc(C(=O)O)cc1 ; C[C@H](NC(=O)c1cc(-c2ccccc2)cn2ccc(Cc3ccc4ccncc4c3)c12)c1ccc(C(=O)O)cc1 ; C[C@H](NC(=O)c1cc(-c2ccccc2)cn2ccc(Cc3ccc4ncccc4c3)c12)c1ccc(C(=O)O)cc1 ; C[C@H](NC(=O)c1cc(-c2cnon2)cn2ccc(Cc3ccc4ccccc4c3)c12)c1ccc(C(=O)O)cc1]

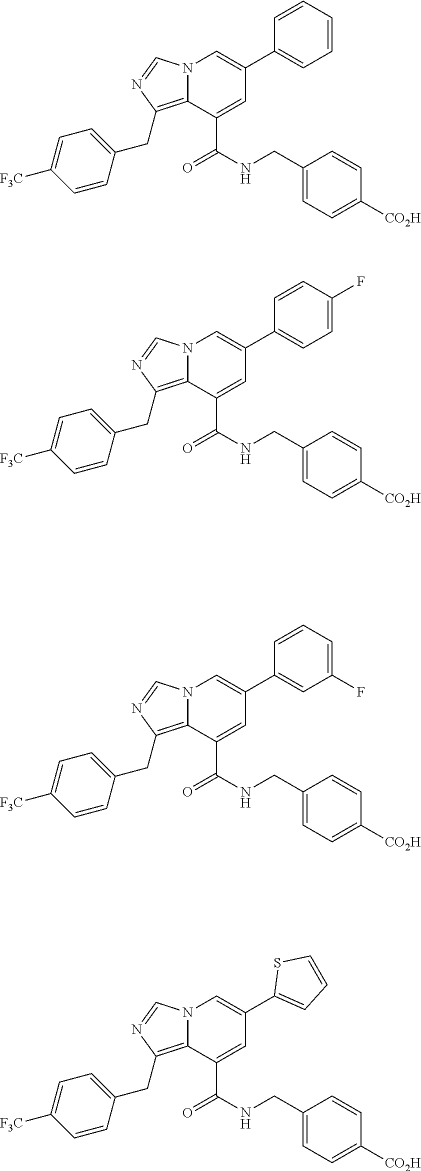 [SMILES: O=C(O)c1ccc(CNC(=O)c2cc(-c3ccccc3)cn3cnc(Cc4ccc(C(F)(F)F)cc4)c23)cc1 ; O=C(O)c1ccc(CNC(=O)c2cc(-c3ccc(F)cc3)cn3cnc(Cc4ccc(C(F)(F)F)cc4)c23)cc1 ; O=C(O)c1ccc(CNC(=O)c2cc(-c3cccc(F)c3)cn3cnc(Cc4ccc(C(F)(F)F)cc4)c23)cc1 ; O=C(O)c1ccc(CNC(=O)c2cc(-c3cccs3)cn3cnc(Cc4ccc(C(F)(F)F)cc4)c23)cc1]

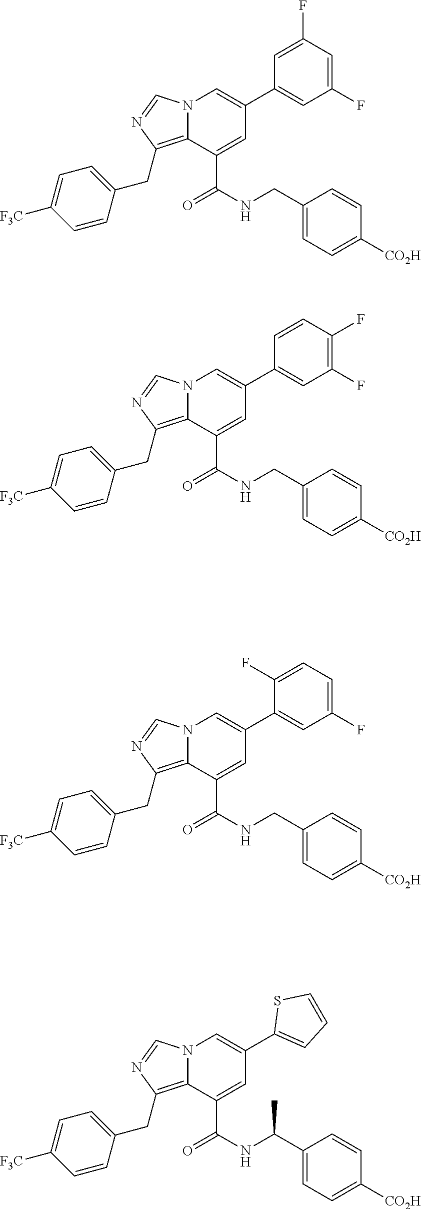 [SMILES: O=C(O)c1ccc(CNC(=O)c2cc(-c3cc(F)cc(F)c3)cn3cnc(Cc4ccc(C(F)(F)F)cc4)c23)cc1 ; O=C(O)c1ccc(CNC(=O)c2cc(-c3ccc(F)c(F)c3)cn3cnc(Cc4ccc(C(F)(F)F)cc4)c23)cc1 ; O=C(O)c1ccc(CNC(=O)c2cc(-c3cc(F)ccc3F)cn3cnc(Cc4ccc(C(F)(F)F)cc4)c23)cc1 ; C[C@H](NC(=O)c1cc(-c2cccs2)cn2cnc(Cc3ccc(C(F)(F)F)cc3)c12)c1ccc(C(=O)O)cc1]

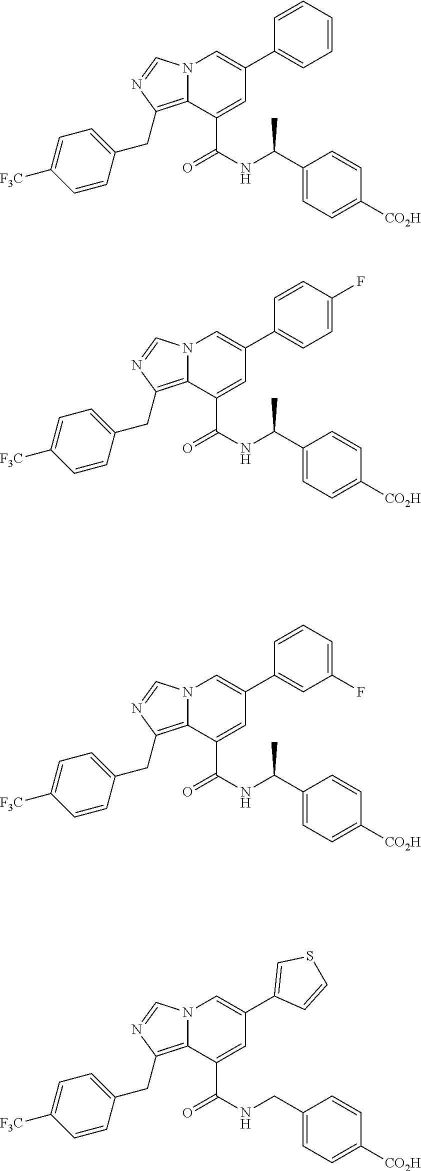 [SMILES: C[C@H](NC(=O)c1cc(-c2ccccc2)cn2cnc(Cc3ccc(C(F)(F)F)cc3)c12)c1ccc(C(=O)O)cc1 ; C[C@H](NC(=O)c1cc(-c2ccc(F)cc2)cn2cnc(Cc3ccc(C(F)(F)F)cc3)c12)c1ccc(C(=O)O)cc1 ; C[C@H](NC(=O)c1cc(-c2cccc(F)c2)cn2cnc(Cc3ccc(C(F)(F)F)cc3)c12)c1ccc(C(=O)O)cc1 ; O=C(O)c1ccc(CNC(=O)c2cc(-c3ccsc3)cn3cnc(Cc4ccc(C(F)(F)F)cc4)c23)cc1]

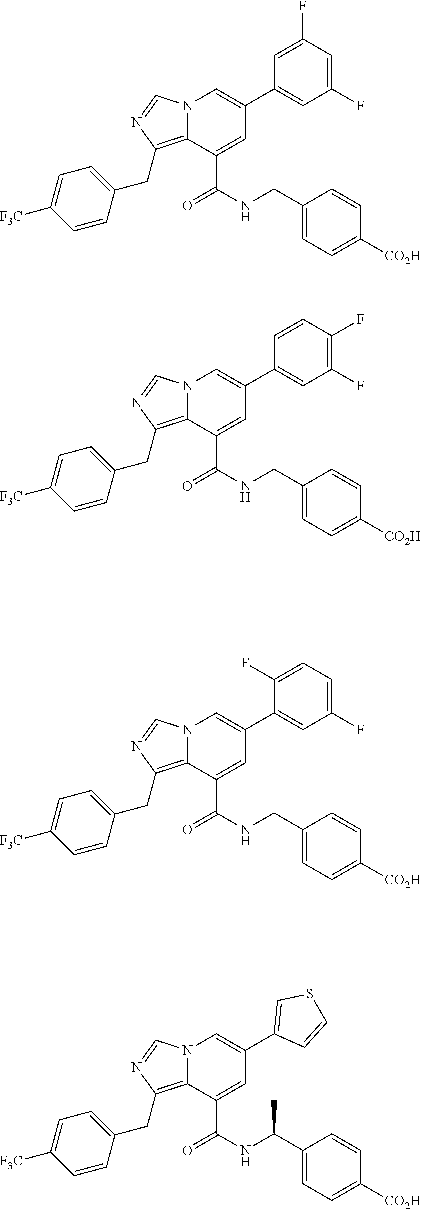 [SMILES: O=C(O)c1ccc(CNC(=O)c2cc(-c3cc(F)cc(F)c3)cn3cnc(Cc4ccc(C(F)(F)F)cc4)c23)cc1 ; O=C(O)c1ccc(CNC(=O)c2cc(-c3ccc(F)c(F)c3)cn3cnc(Cc4ccc(C(F)(F)F)cc4)c23)cc1 ; O=C(O)c1ccc(CNC(=O)c2cc(-c3cc(F)ccc3F)cn3cnc(Cc4ccc(C(F)(F)F)cc4)c23)cc1 ; C[C@H](NC(=O)c1cc(-c2ccsc2)cn2cnc(Cc3ccc(C(F)(F)F)cc3)c12)c1ccc(C(=O)O)cc1]

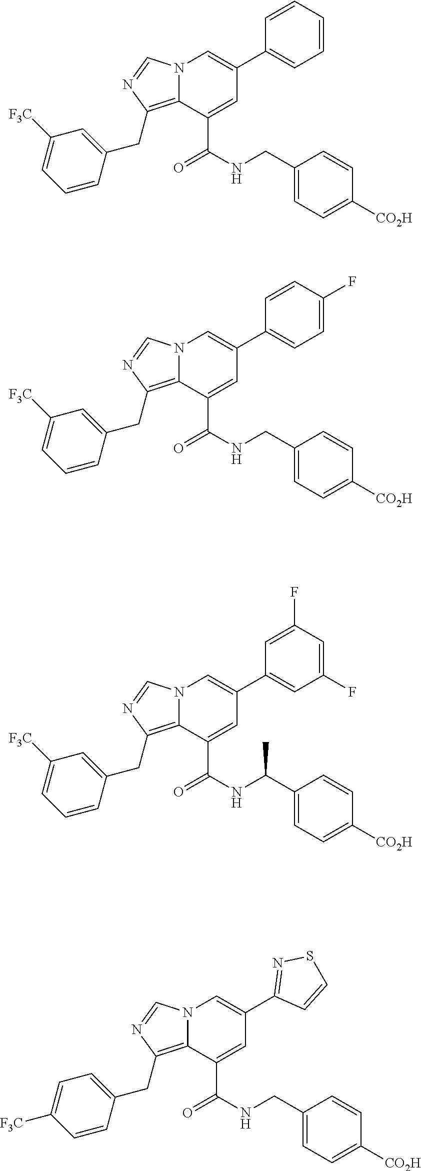 [SMILES: O=C(O)c1ccc(CNC(=O)c2cc(-c3ccccc3)cn3cnc(Cc4cccc(C(F)(F)F)c4)c23)cc1 ; O=C(O)c1ccc(CNC(=O)c2cc(-c3ccc(F)cc3)cn3cnc(Cc4cccc(C(F)(F)F)c4)c23)cc1 ; C[C@H](NC(=O)c1cc(-c2cc(F)cc(F)c2)cn2cnc(Cc3cccc(C(F)(F)F)c3)c12)c1ccc(C(=O)O)cc1 ; O=C(O)c1ccc(CNC(=O)c2cc(-c3ccsn3)cn3cnc(Cc4ccc(C(F)(F)F)cc4)c23)cc1]

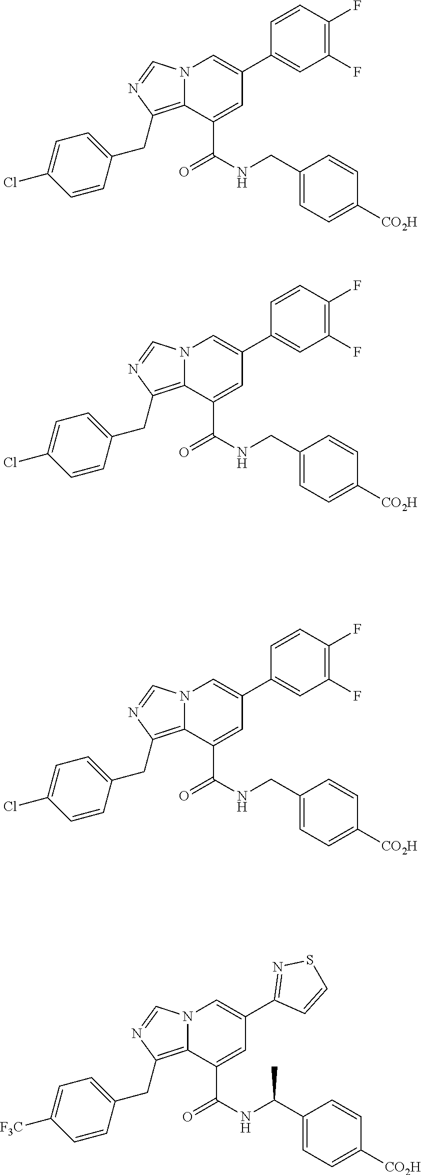 [SMILES: O=C(O)c1ccc(CNC(=O)c2cc(-c3ccc(F)c(F)c3)cn3cnc(Cc4ccc(Cl)cc4)c23)cc1 ; O=C(O)c1ccc(CNC(=O)c2cc(-c3ccc(F)c(F)c3)cn3cnc(Cc4ccc(Cl)cc4)c23)cc1 ; O=C(O)c1ccc(CNC(=O)c2cc(-c3ccc(F)c(F)c3)cn3cnc(Cc4ccc(Cl)cc4)c23)cc1 ; C[C@H](NC(=O)c1cc(-c2ccsn2)cn2cnc(Cc3ccc(C(F)(F)F)cc3)c12)c1ccc(C(=O)O)cc1]

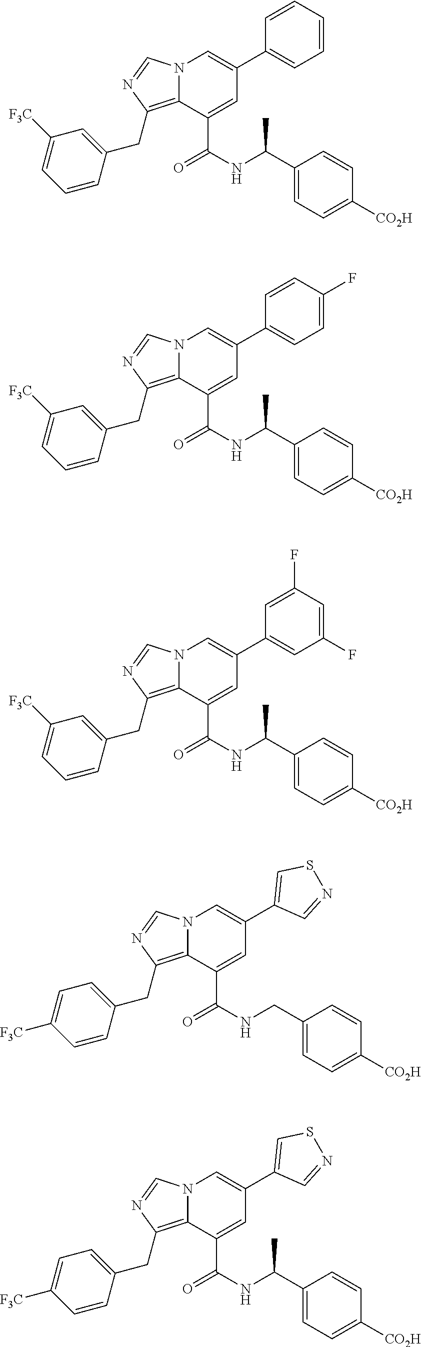 [SMILES: C[C@H](NC(=O)c1cc(-c2ccccc2)cn2cnc(Cc3cccc(C(F)(F)F)c3)c12)c1ccc(C(=O)O)cc1 ; C[C@H](NC(=O)c1cc(-c2ccc(F)cc2)cn2cnc(Cc3cccc(C(F)(F)F)c3)c12)c1ccc(C(=O)O)cc1 ; C[C@H](NC(=O)c1cc(-c2cc(F)cc(F)c2)cn2cnc(Cc3cccc(C(F)(F)F)c3)c12)c1ccc(C(=O)O)cc1 ; O=C(O)c1ccc(CNC(=O)c2cc(-c3cnsc3)cn3cnc(Cc4ccc(C(F)(F)F)cc4)c23)cc1 ; C[C@H](NC(=O)c1cc(-c2cnsc2)cn2cnc(Cc3ccc(C(F)(F)F)cc3)c12)c1ccc(C(=O)O)cc1]

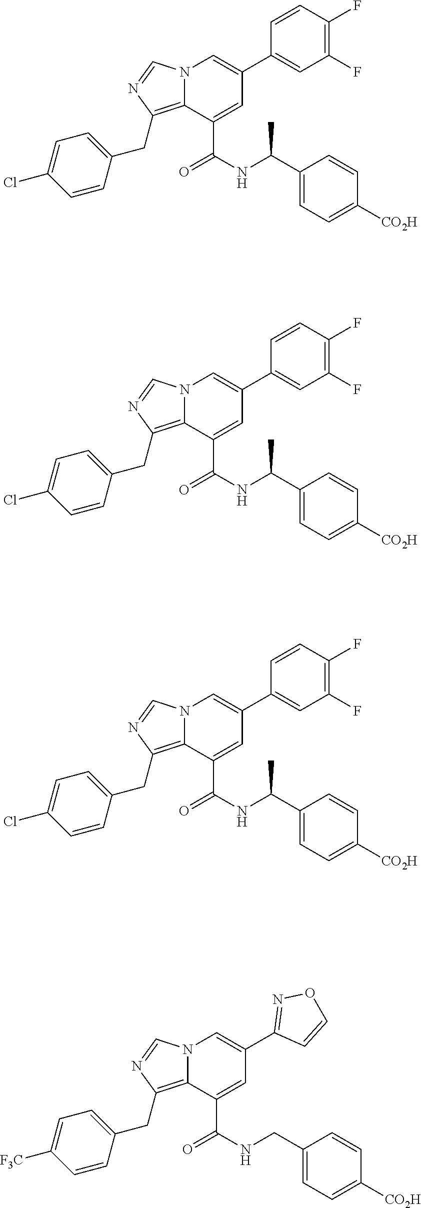 [SMILES: C[C@H](NC(=O)c1cc(-c2ccc(F)c(F)c2)cn2cnc(Cc3ccc(Cl)cc3)c12)c1ccc(C(=O)O)cc1 ; C[C@H](NC(=O)c1cc(-c2ccc(F)c(F)c2)cn2cnc(Cc3ccc(Cl)cc3)c12)c1ccc(C(=O)O)cc1 ; C[C@H](NC(=O)c1cc(-c2ccc(F)c(F)c2)cn2cnc(Cc3ccc(Cl)cc3)c12)c1ccc(C(=O)O)cc1 ; O=C(O)c1ccc(CNC(=O)c2cc(-c3ccon3)cn3cnc(Cc4ccc(C(F)(F)F)cc4)c23)cc1]

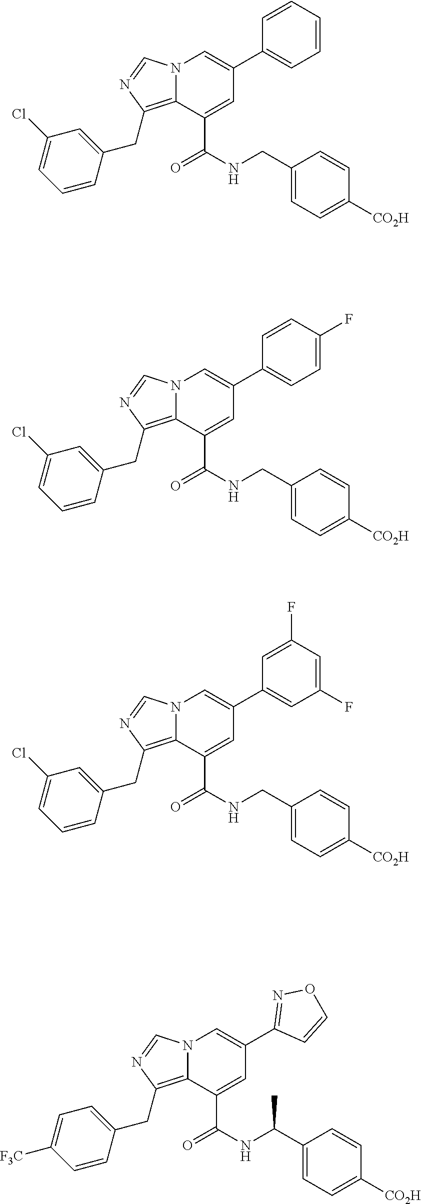 [SMILES: O=C(O)c1ccc(CNC(=O)c2cc(-c3ccccc3)cn3cnc(Cc4cccc(Cl)c4)c23)cc1 ; O=C(O)c1ccc(CNC(=O)c2cc(-c3ccc(F)cc3)cn3cnc(Cc4cccc(Cl)c4)c23)cc1 ; O=C(O)c1ccc(CNC(=O)c2cc(-c3cc(F)cc(F)c3)cn3cnc(Cc4cccc(Cl)c4)c23)cc1 ; C[C@H](NC(=O)c1cc(-c2ccon2)cn2cnc(Cc3ccc(C(F)(F)F)cc3)c12)c1ccc(C(=O)O)cc1]

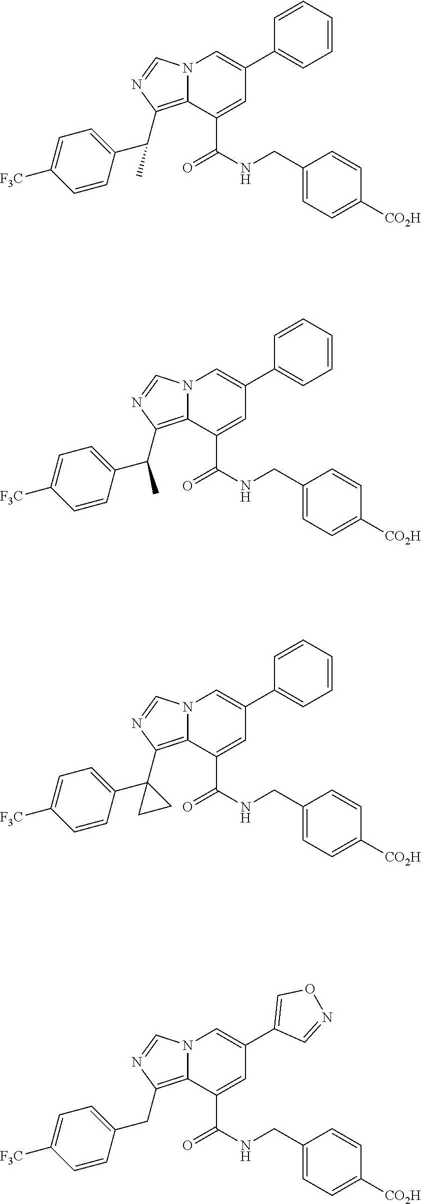 [SMILES: C[C@H](c1ccc(C(F)(F)F)cc1)c1ncn2cc(-c3ccccc3)cc(C(=O)NCc3ccc(C(=O)O)cc3)c12 ; C[C@@H](c1ccc(C(F)(F)F)cc1)c1ncn2cc(-c3ccccc3)cc(C(=O)NCc3ccc(C(=O)O)cc3)c12 ; O=C(O)c1ccc(CNC(=O)c2cc(-c3ccccc3)cn3cnc(C4(c5ccc(C(F)(F)F)cc5)CC4)c23)cc1 ; O=C(O)c1ccc(CNC(=O)c2cc(-c3cnoc3)cn3cnc(Cc4ccc(C(F)(F)F)cc4)c23)cc1]

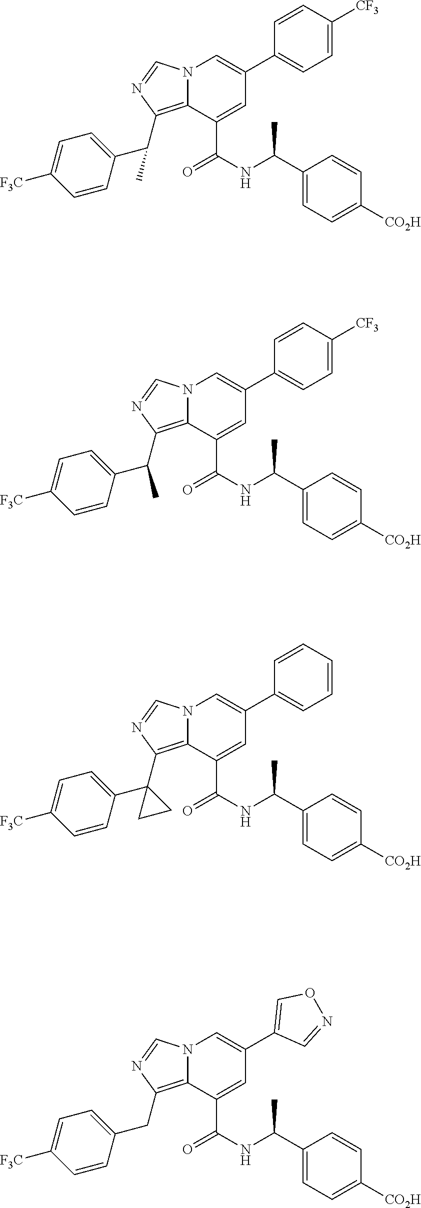 [SMILES: C[C@H](c1ccc(C(F)(F)F)cc1)c1ncn2cc(-c3ccc(C(F)(F)F)cc3)cc(C(=O)N[C@@H](C)c3ccc(C(=O)O)cc3)c12 ; C[C@@H](c1ccc(C(F)(F)F)cc1)c1ncn2cc(-c3ccc(C(F)(F)F)cc3)cc(C(=O)N[C@@H](C)c3ccc(C(=O)O)cc3)c12 ; C[C@H](NC(=O)c1cc(-c2ccccc2)cn2cnc(C3(c4ccc(C(F)(F)F)cc4)CC3)c12)c1ccc(C(=O)O)cc1 ; C[C@H](NC(=O)c1cc(-c2cnoc2)cn2cnc(Cc3ccc(C(F)(F)F)cc3)c12)c1ccc(C(=O)O)cc1]

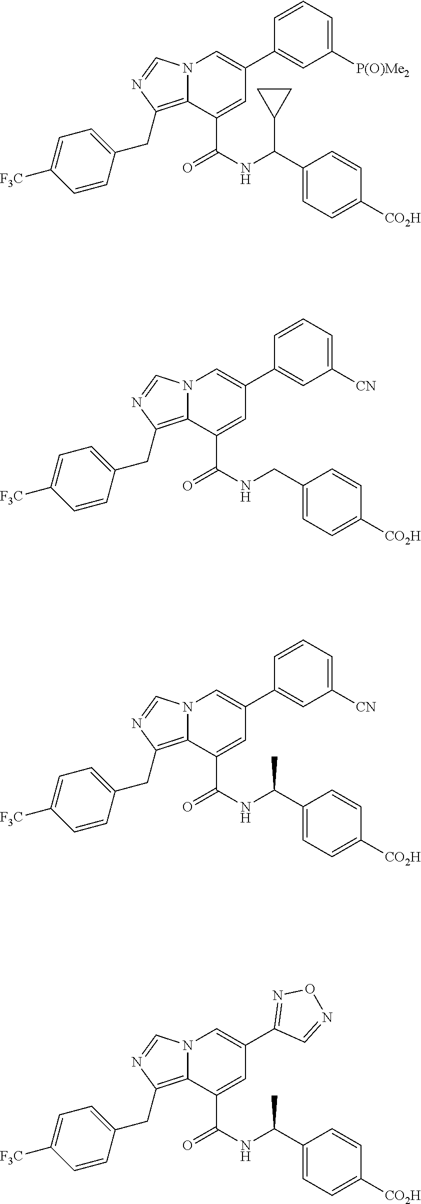

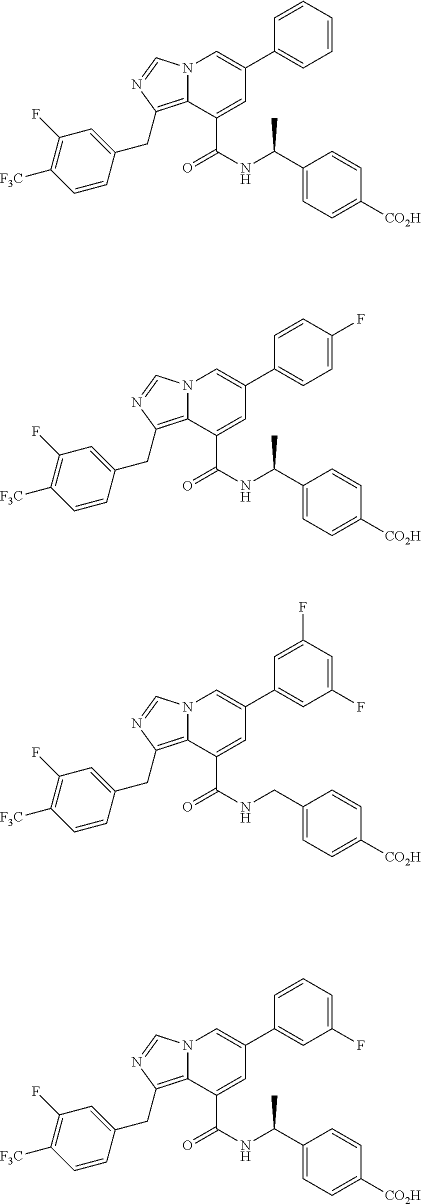 [SMILES: C[C@H](NC(=O)c1cc(-c2ccccc2)cn2cnc(Cc3ccc(C(F)(F)F)c(F)c3)c12)c1ccc(C(=O)O)cc1 ; C[C@H](NC(=O)c1cc(-c2ccc(F)cc2)cn2cnc(Cc3ccc(C(F)(F)F)c(F)c3)c12)c1ccc(C(=O)O)cc1 ; O=C(O)c1ccc(CNC(=O)c2cc(-c3cc(F)cc(F)c3)cn3cnc(Cc4ccc(C(F)(F)F)c(F)c4)c23)cc1 ; C[C@H](NC(=O)c1cc(-c2cccc(F)c2)cn2cnc(Cc3ccc(C(F)(F)F)c(F)c3)c12)c1ccc(C(=O)O)cc1]

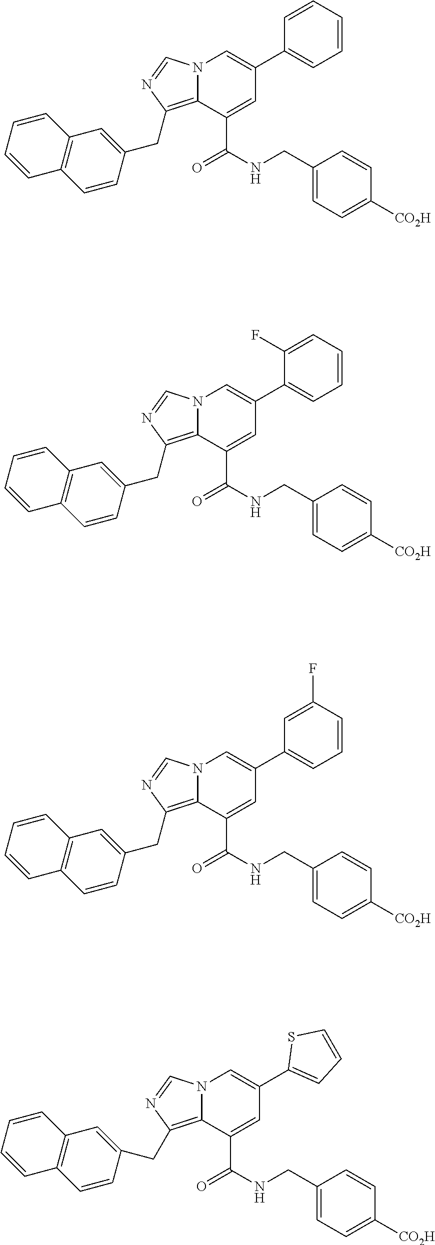 [SMILES: O=C(O)c1ccc(CNC(=O)c2cc(-c3ccccc3)cn3cnc(Cc4ccc5ccccc5c4)c23)cc1 ; O=C(O)c1ccc(CNC(=O)c2cc(-c3ccccc3F)cn3cnc(Cc4ccc5ccccc5c4)c23)cc1 ; O=C(O)c1ccc(CNC(=O)c2cc(-c3cccc(F)c3)cn3cnc(Cc4ccc5ccccc5c4)c23)cc1 ; O=C(O)c1ccc(CNC(=O)c2cc(-c3cccs3)cn3cnc(Cc4ccc5ccccc5c4)c23)cc1]

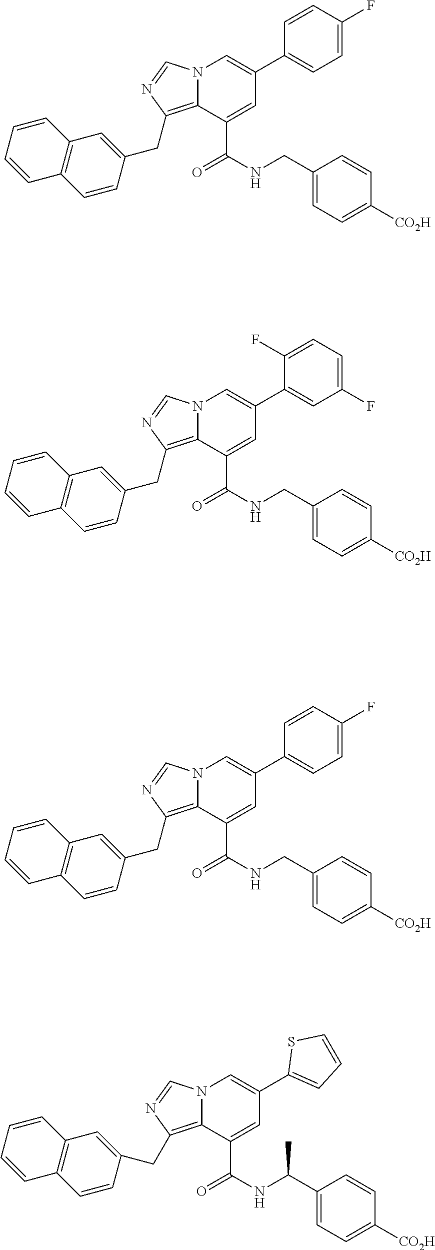 [SMILES: O=C(O)c1ccc(CNC(=O)c2cc(-c3ccc(F)cc3)cn3cnc(Cc4ccc5ccccc5c4)c23)cc1 ; O=C(O)c1ccc(CNC(=O)c2cc(-c3cc(F)ccc3F)cn3cnc(Cc4ccc5ccccc5c4)c23)cc1 ; O=C(O)c1ccc(CNC(=O)c2cc(-c3ccc(F)cc3)cn3cnc(Cc4ccc5ccccc5c4)c23)cc1 ; C[C@H](NC(=O)c1cc(-c2cccs2)cn2cnc(Cc3ccc4ccccc4c3)c12)c1ccc(C(=O)O)cc1]

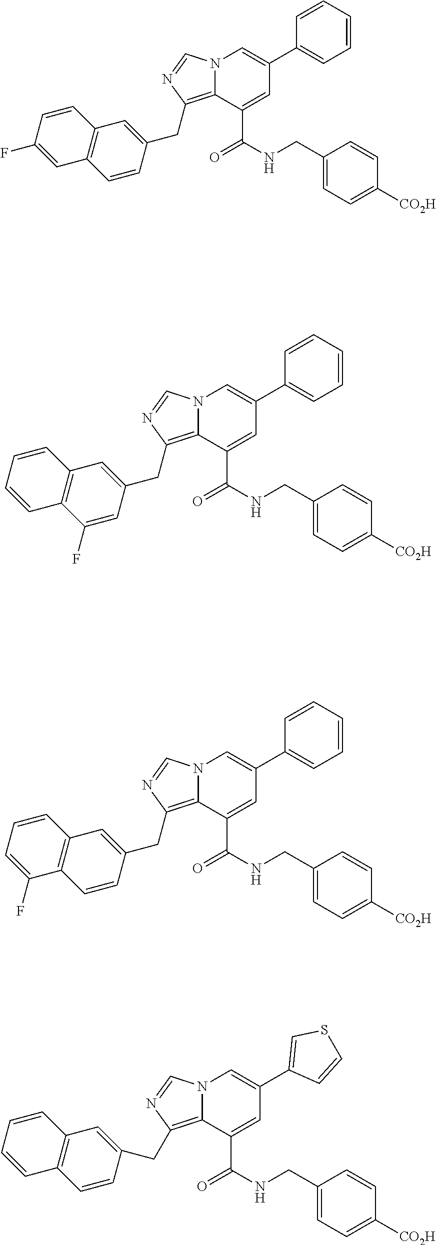 [SMILES: O=C(O)c1ccc(CNC(=O)c2cc(-c3ccccc3)cn3cnc(Cc4ccc5cc(F)ccc5c4)c23)cc1 ; O=C(O)c1ccc(CNC(=O)c2cc(-c3ccccc3)cn3cnc(Cc4cc(F)c5ccccc5c4)c23)cc1 ; O=C(O)c1ccc(CNC(=O)c2cc(-c3ccccc3)cn3cnc(Cc4ccc5c(F)cccc5c4)c23)cc1 ; O=C(O)c1ccc(CNC(=O)c2cc(-c3ccsc3)cn3cnc(Cc4ccc5ccccc5c4)c23)cc1]

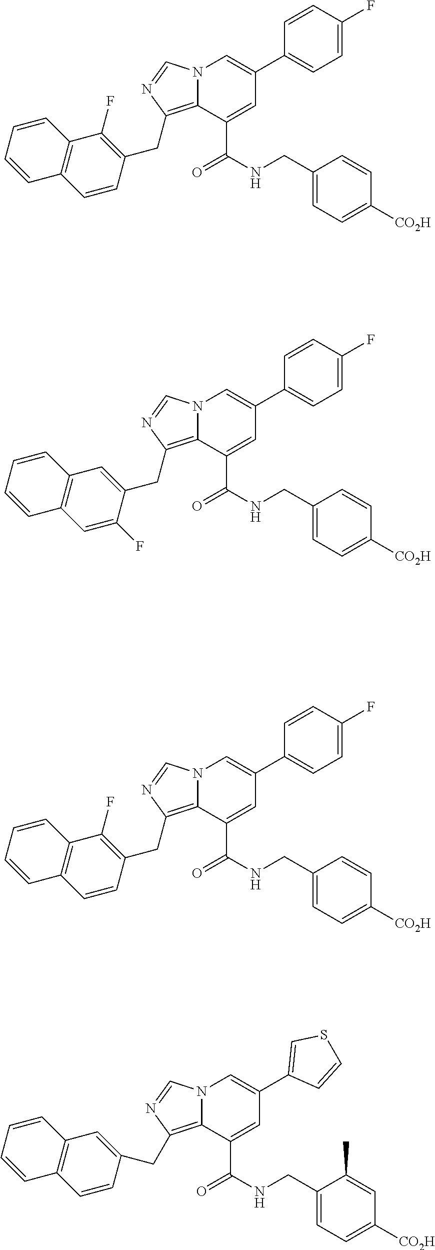

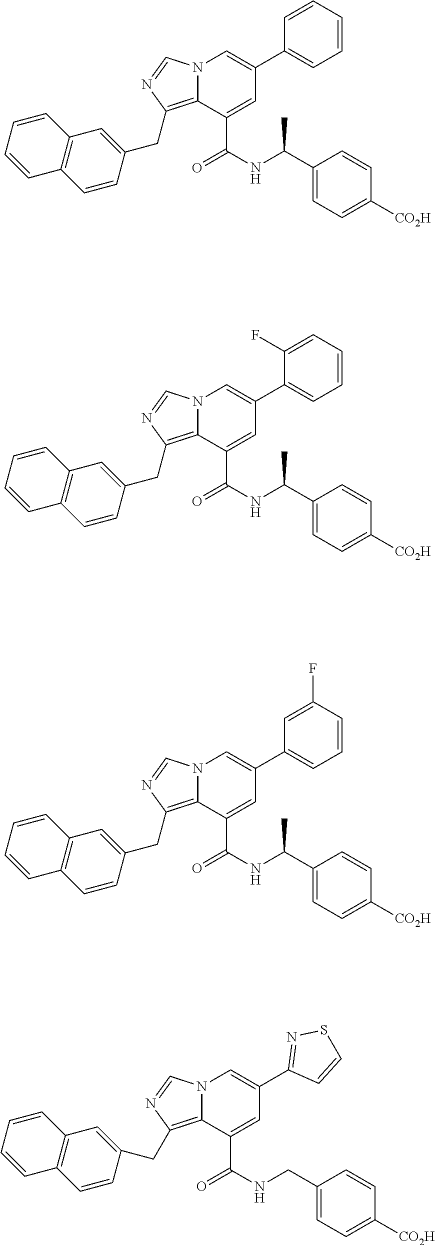 [SMILES: C[C@H](NC(=O)c1cc(-c2ccccc2)cn2cnc(Cc3ccc4ccccc4c3)c12)c1ccc(C(=O)O)cc1 ; C[C@H](NC(=O)c1cc(-c2ccccc2F)cn2cnc(Cc3ccc4ccccc4c3)c12)c1ccc(C(=O)O)cc1 ; C[C@H](NC(=O)c1cc(-c2cccc(F)c2)cn2cnc(Cc3ccc4ccccc4c3)c12)c1ccc(C(=O)O)cc1 ; O=C(O)c1ccc(CNC(=O)c2cc(-c3ccsn3)cn3cnc(Cc4ccc5ccccc5c4)c23)cc1]

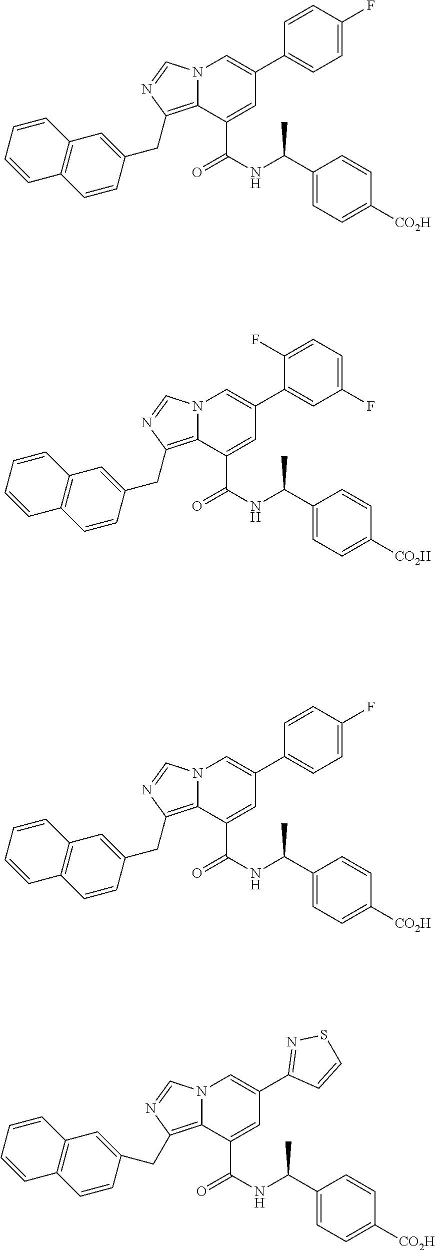 [SMILES: C[C@H](NC(=O)c1cc(-c2ccc(F)cc2)cn2cnc(Cc3ccc4ccccc4c3)c12)c1ccc(C(=O)O)cc1 ; C[C@H](NC(=O)c1cc(-c2cc(F)ccc2F)cn2cnc(Cc3ccc4ccccc4c3)c12)c1ccc(C(=O)O)cc1 ; C[C@H](NC(=O)c1cc(-c2ccc(F)cc2)cn2cnc(Cc3ccc4ccccc4c3)c12)c1ccc(C(=O)O)cc1 ; C[C@H](NC(=O)c1cc(-c2ccsn2)cn2cnc(Cc3ccc4ccccc4c3)c12)c1ccc(C(=O)O)cc1]

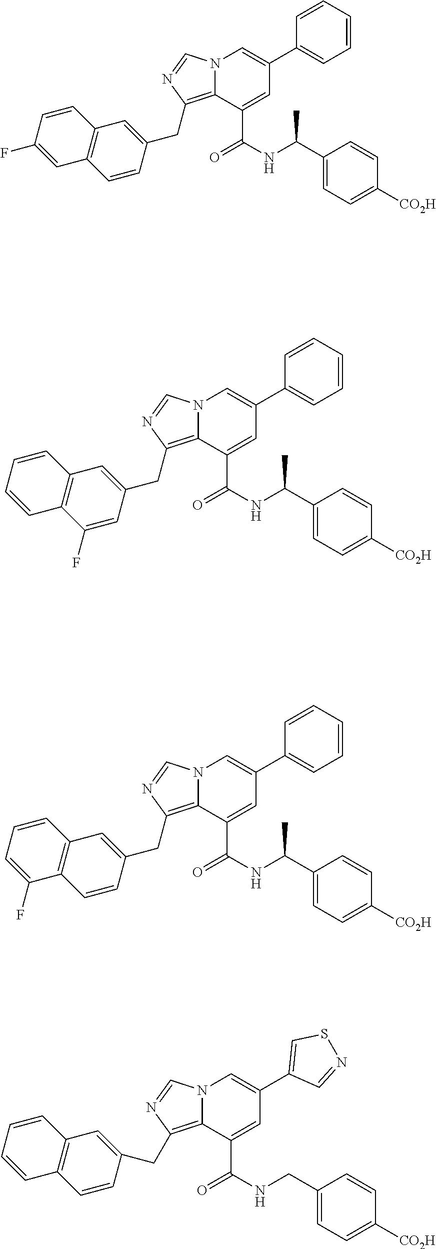 [SMILES: C[C@H](NC(=O)c1cc(-c2ccccc2)cn2cnc(Cc3ccc4cc(F)ccc4c3)c12)c1ccc(C(=O)O)cc1 ; C[C@H](NC(=O)c1cc(-c2ccccc2)cn2cnc(Cc3cc(F)c4ccccc4c3)c12)c1ccc(C(=O)O)cc1 ; C[C@H](NC(=O)c1cc(-c2ccccc2)cn2cnc(Cc3ccc4c(F)cccc4c3)c12)c1ccc(C(=O)O)cc1 ; O=C(O)c1ccc(CNC(=O)c2cc(-c3cnsc3)cn3cnc(Cc4ccc5ccccc5c4)c23)cc1]

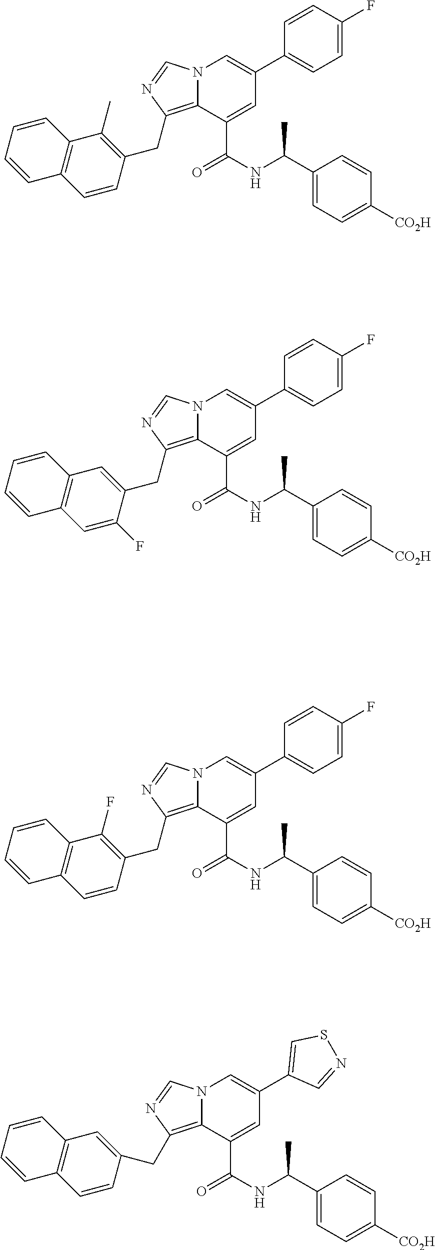 [SMILES: Cc1c(Cc2ncn3cc(-c4ccc(F)cc4)cc(C(=O)N[C@@H](C)c4ccc(C(=O)O)cc4)c23)ccc2ccccc12 ; C[C@H](NC(=O)c1cc(-c2ccc(F)cc2)cn2cnc(Cc3cc4ccccc4cc3F)c12)c1ccc(C(=O)O)cc1 ; C[C@H](NC(=O)c1cc(-c2ccc(F)cc2)cn2cnc(Cc3ccc4ccccc4c3F)c12)c1ccc(C(=O)O)cc1 ; C[C@H](NC(=O)c1cc(-c2cnsc2)cn2cnc(Cc3ccc4ccccc4c3)c12)c1ccc(C(=O)O)cc1]

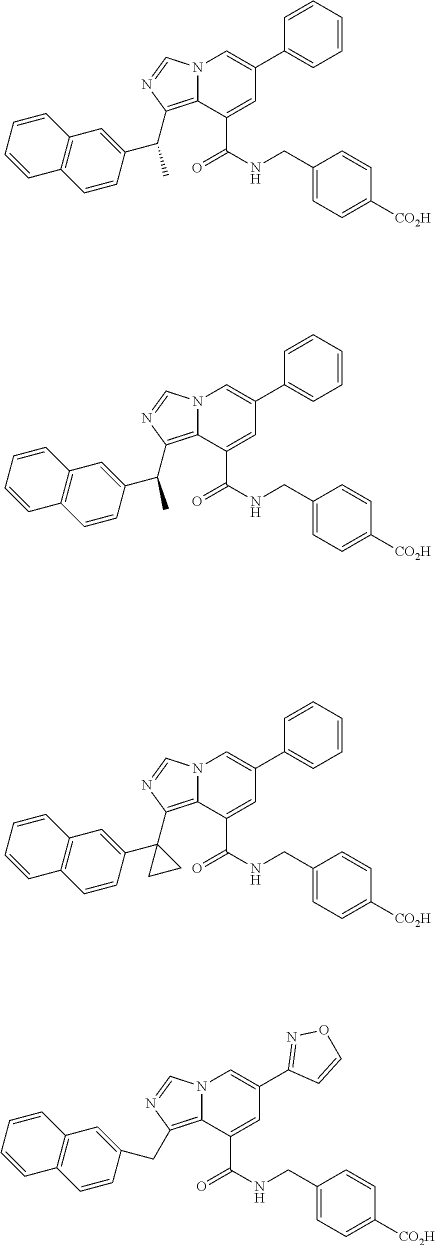 [SMILES: C[C@H](c1ccc2ccccc2c1)c1ncn2cc(-c3ccccc3)cc(C(=O)NCc3ccc(C(=O)O)cc3)c12 ; C[C@@H](c1ccc2ccccc2c1)c1ncn2cc(-c3ccccc3)cc(C(=O)NCc3ccc(C(=O)O)cc3)c12 ; O=C(O)c1ccc(CNC(=O)c2cc(-c3ccccc3)cn3cnc(C4(c5ccc6ccccc6c5)CC4)c23)cc1 ; O=C(O)c1ccc(CNC(=O)c2cc(-c3ccon3)cn3cnc(Cc4ccc5ccccc5c4)c23)cc1]

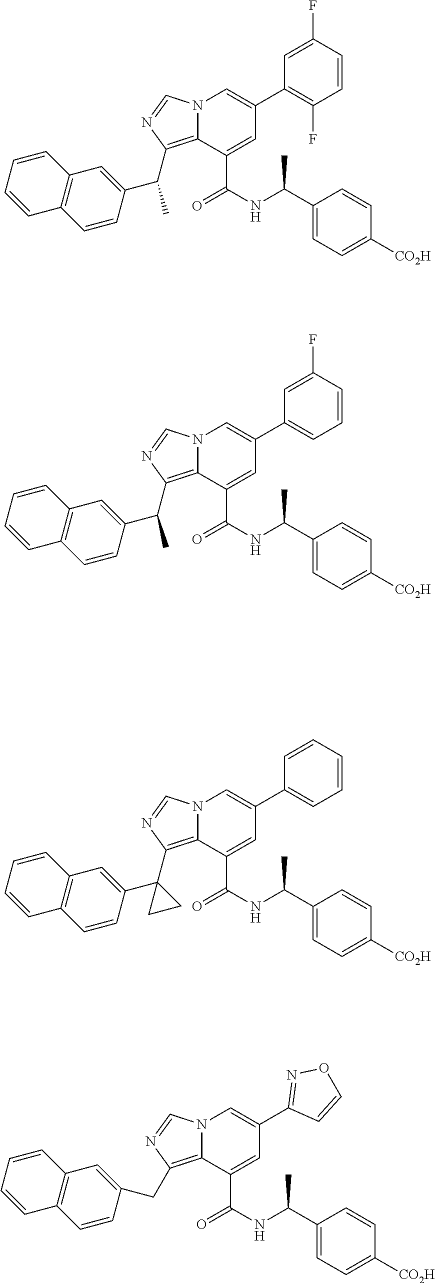 [SMILES: C[C@H](c1ccc2ccccc2c1)c1ncn2cc(-c3cc(F)ccc3F)cc(C(=O)N[C@@H](C)c3ccc(C(=O)O)cc3)c12 ; C[C@@H](c1ccc2ccccc2c1)c1ncn2cc(-c3cccc(F)c3)cc(C(=O)N[C@@H](C)c3ccc(C(=O)O)cc3)c12 ; C[C@H](NC(=O)c1cc(-c2ccccc2)cn2cnc(C3(c4ccc5ccccc5c4)CC3)c12)c1ccc(C(=O)O)cc1 ; C[C@H](NC(=O)c1cc(-c2ccon2)cn2cnc(Cc3ccc4ccccc4c3)c12)c1ccc(C(=O)O)cc1]

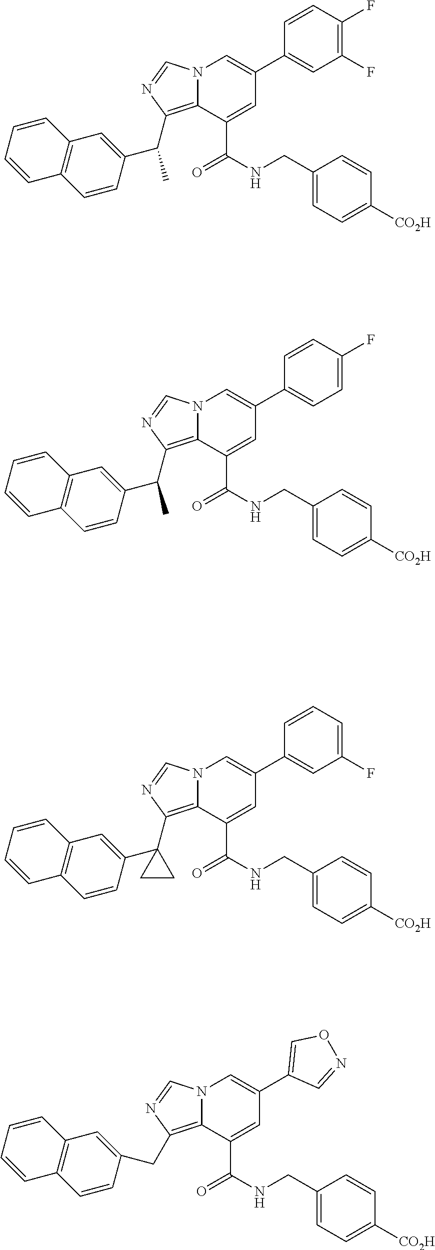 [SMILES: C[C@H](c1ccc2ccccc2c1)c1ncn2cc(-c3ccc(F)c(F)c3)cc(C(=O)NCc3ccc(C(=O)O)cc3)c12 ; C[C@@H](c1ccc2ccccc2c1)c1ncn2cc(-c3ccc(F)cc3)cc(C(=O)NCc3ccc(C(=O)O)cc3)c12 ; O=C(O)c1ccc(CNC(=O)c2cc(-c3cccc(F)c3)cn3cnc(C4(c5ccc6ccccc6c5)CC4)c23)cc1 ; O=C(O)c1ccc(CNC(=O)c2cc(-c3cnoc3)cn3cnc(Cc4ccc5ccccc5c4)c23)cc1]

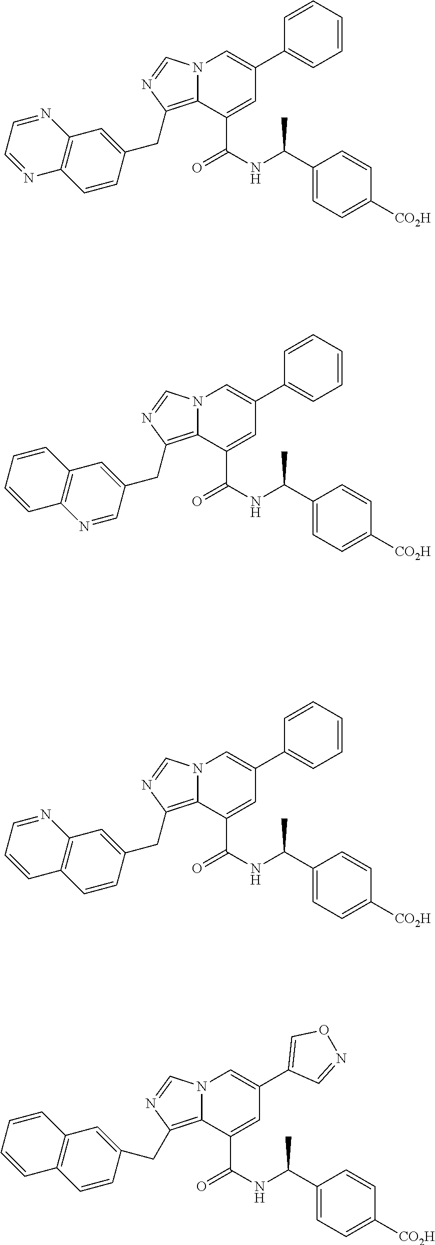 [SMILES: C[C@H](NC(=O)c1cc(-c2ccccc2)cn2cnc(Cc3ccc4nccnc4c3)c12)c1ccc(C(=O)O)cc1 ; C[C@H](NC(=O)c1cc(-c2ccccc2)cn2cnc(Cc3cnc4ccccc4c3)c12)c1ccc(C(=O)O)cc1 ; C[C@H](NC(=O)c1cc(-c2ccccc2)cn2cnc(Cc3ccc4cccnc4c3)c12)c1ccc(C(=O)O)cc1 ; C[C@H](NC(=O)c1cc(-c2cnoc2)cn2cnc(Cc3ccc4ccccc4c3)c12)c1ccc(C(=O)O)cc1]

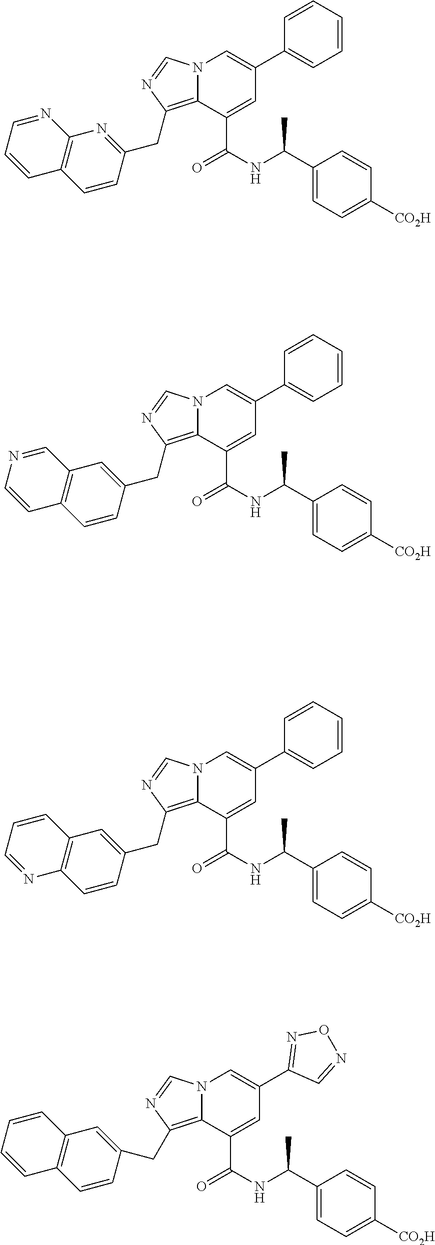 [SMILES: C[C@H](NC(=O)c1cc(-c2ccccc2)cn2cnc(Cc3ccc4cccnc4n3)c12)c1ccc(C(=O)O)cc1 ; C[C@H](NC(=O)c1cc(-c2ccccc2)cn2cnc(Cc3ccc4ccncc4c3)c12)c1ccc(C(=O)O)cc1 ; C[C@H](NC(=O)c1cc(-c2ccccc2)cn2cnc(Cc3ccc4ncccc4c3)c12)c1ccc(C(=O)O)cc1 ; C[C@H](NC(=O)c1cc(-c2cnon2)cn2cnc(Cc3ccc4ccccc4c3)c12)c1ccc(C(=O)O)cc1]

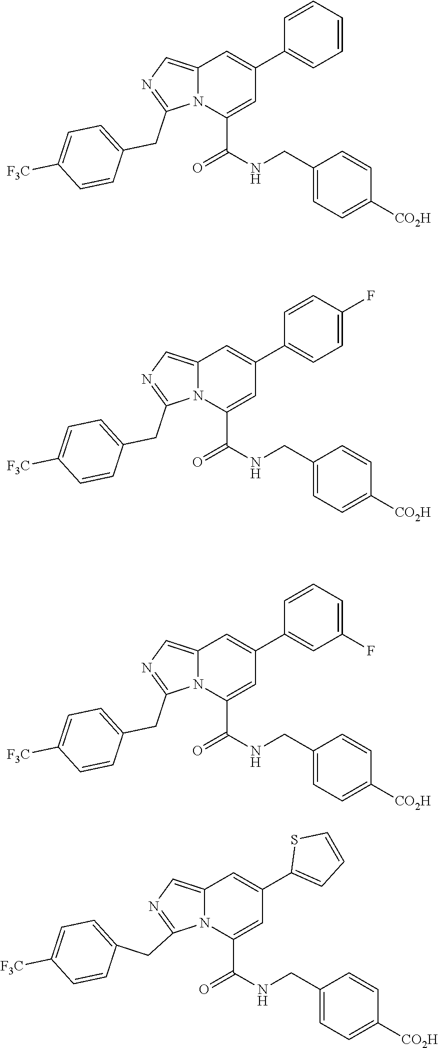

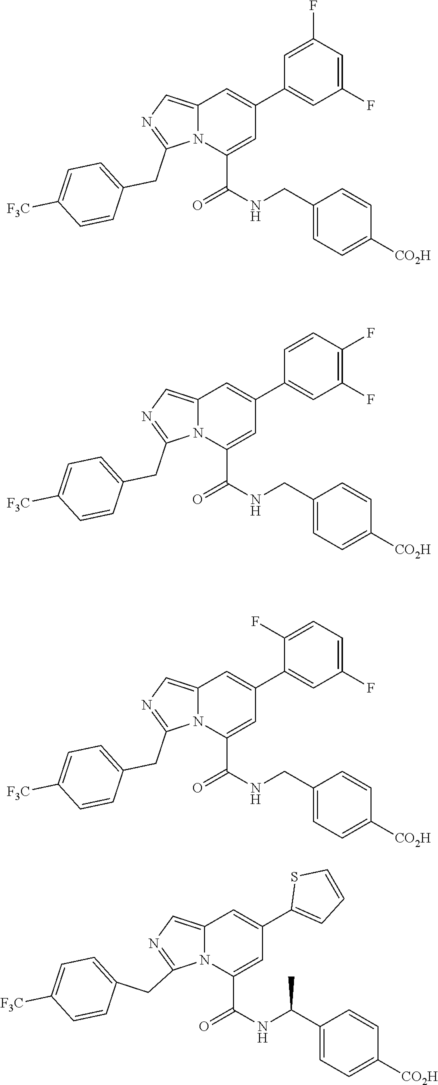

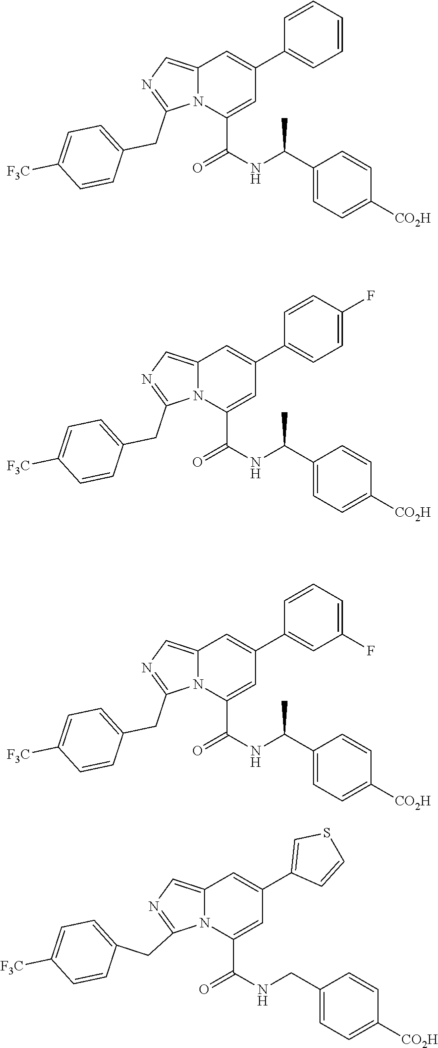

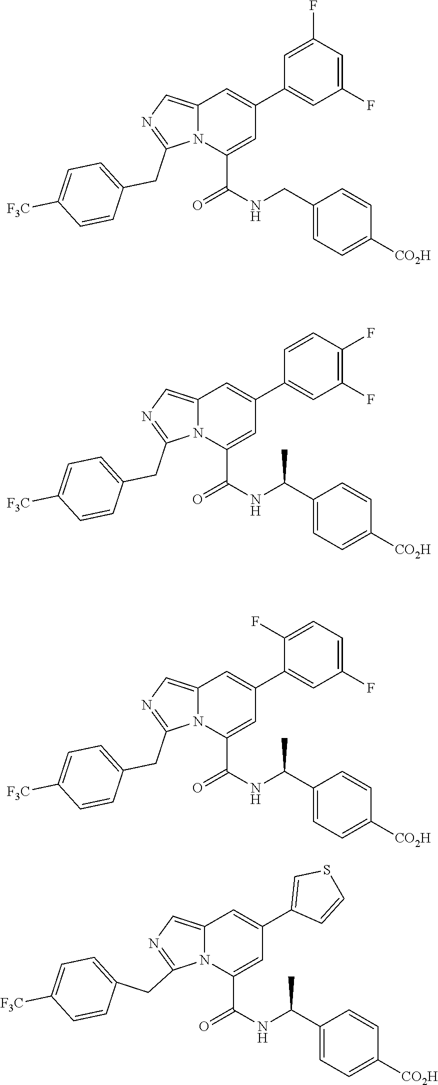

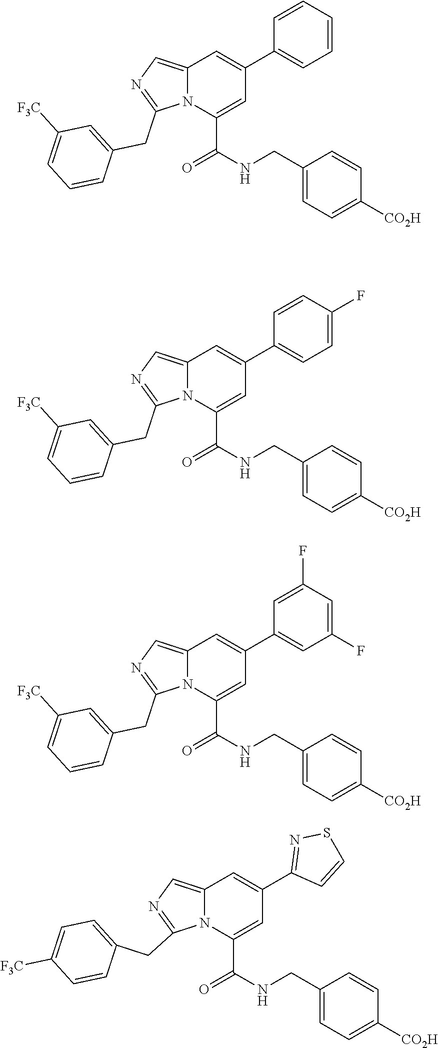

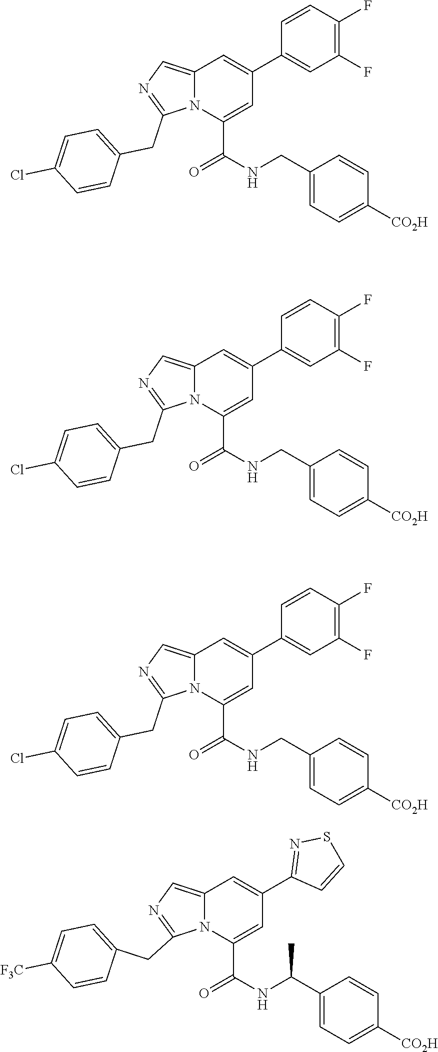

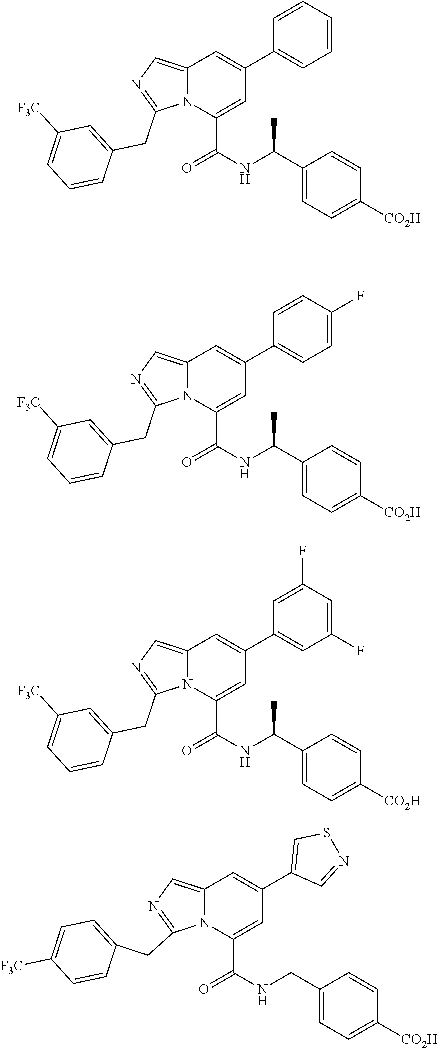

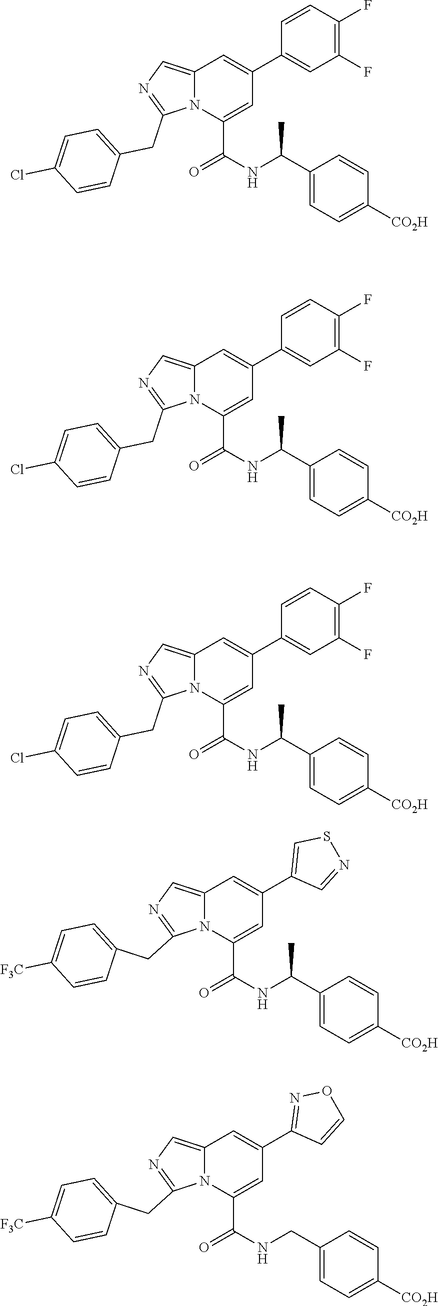

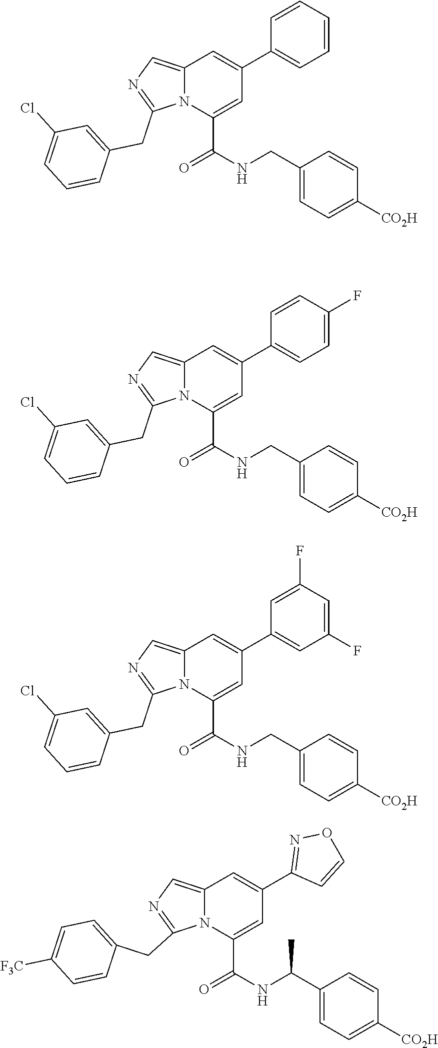

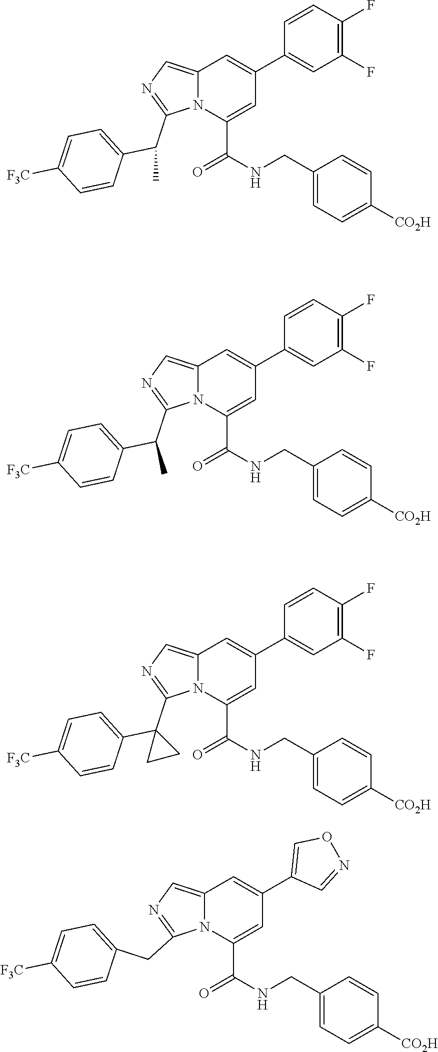

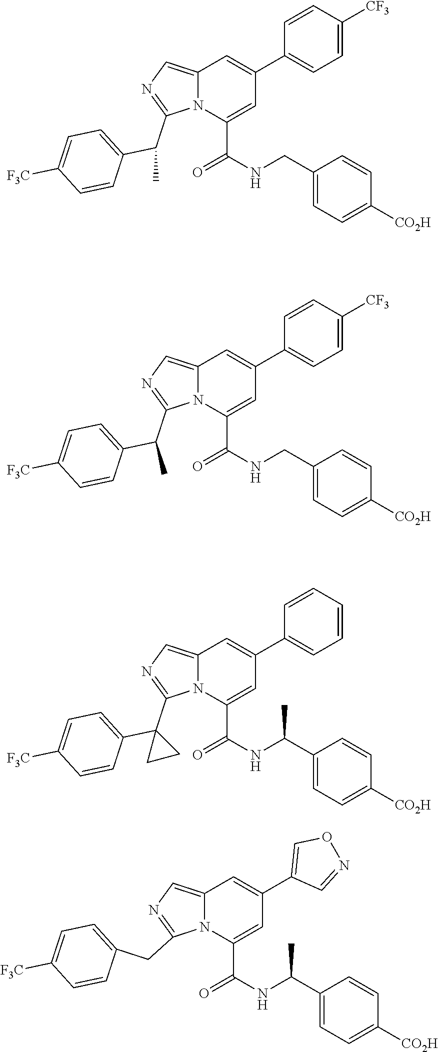

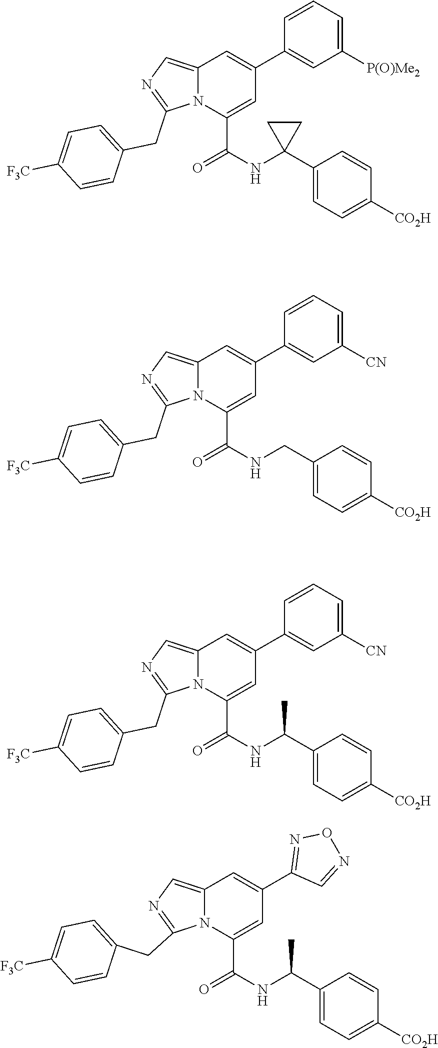

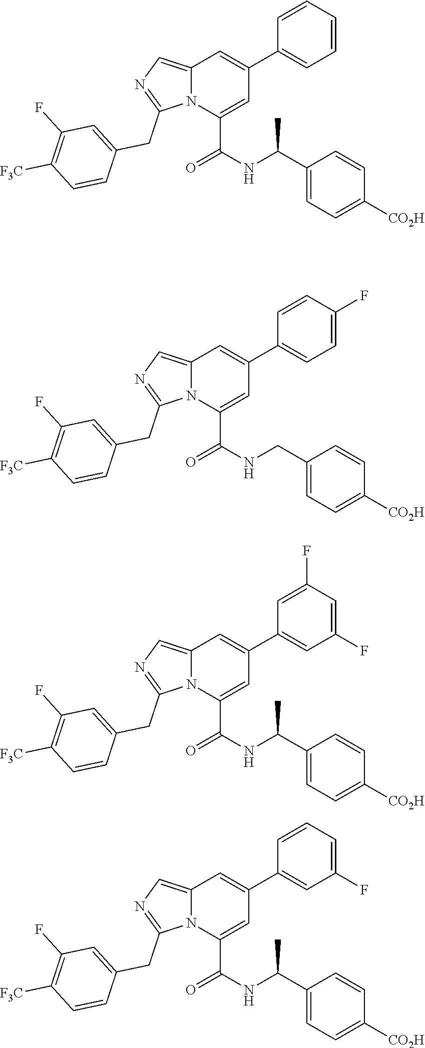

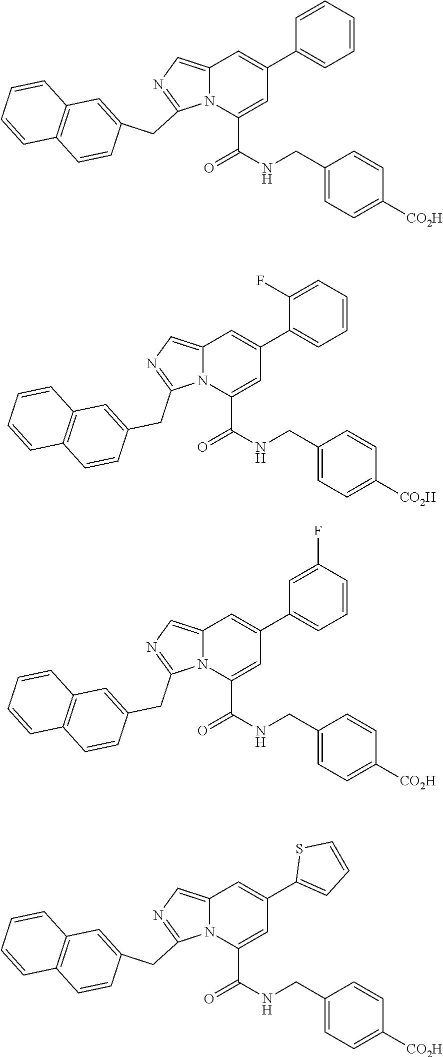

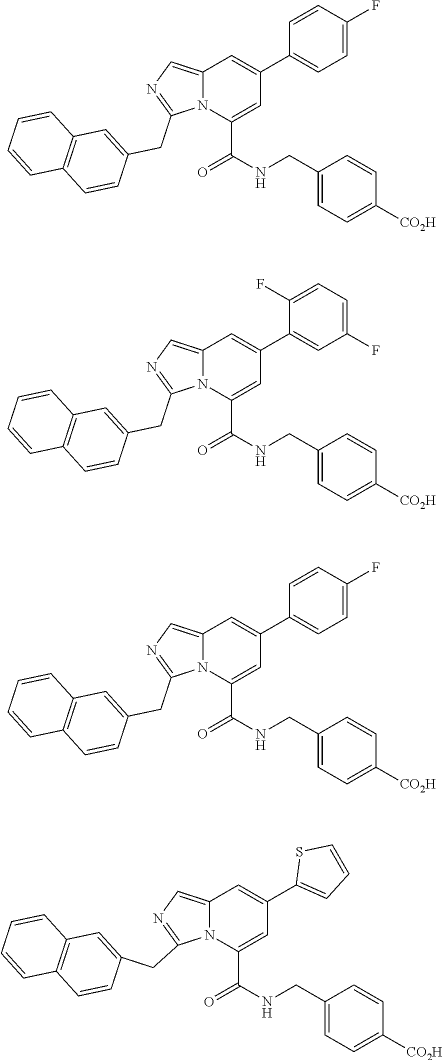 [SMILES: O=C(O)c1ccc(CNC(=O)c2cc(-c3ccc(F)cc3)cc3cnc(Cc4ccc5ccccc5c4)n23)cc1 ; O=C(O)c1ccc(CNC(=O)c2cc(-c3cc(F)ccc3F)cc3cnc(Cc4ccc5ccccc5c4)n23)cc1 ; O=C(O)c1ccc(CNC(=O)c2cc(-c3ccc(F)cc3)cc3cnc(Cc4ccc5ccccc5c4)n23)cc1 ; O=C(O)c1ccc(CNC(=O)c2cc(-c3cccs3)cc3cnc(Cc4ccc5ccccc5c4)n23)cc1]

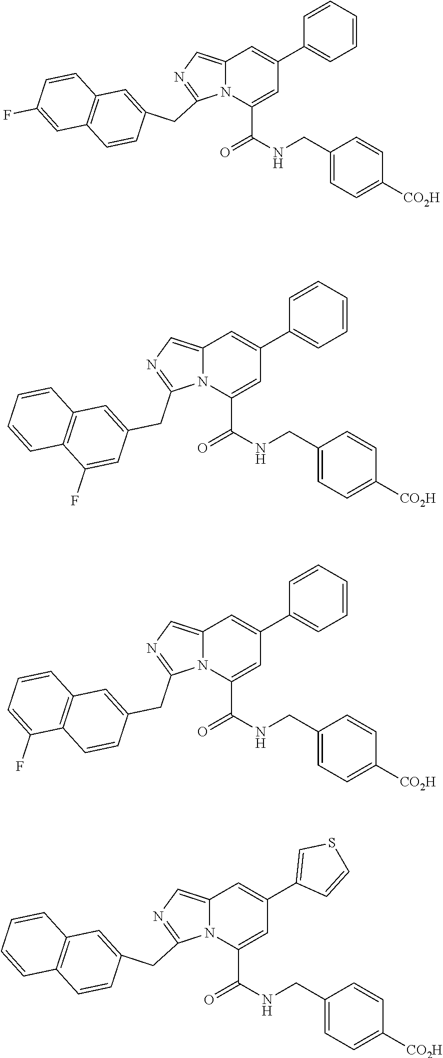 [SMILES: O=C(O)c1ccc(CNC(=O)c2cc(-c3ccccc3)cc3cnc(Cc4ccc5cc(F)ccc5c4)n23)cc1 ; O=C(O)c1ccc(CNC(=O)c2cc(-c3ccccc3)cc3cnc(Cc4cc(F)c5ccccc5c4)n23)cc1 ; O=C(O)c1ccc(CNC(=O)c2cc(-c3ccccc3)cc3cnc(Cc4ccc5c(F)cccc5c4)n23)cc1 ; O=C(O)c1ccc(CNC(=O)c2cc(-c3ccsc3)cc3cnc(Cc4ccc5ccccc5c4)n23)cc1]

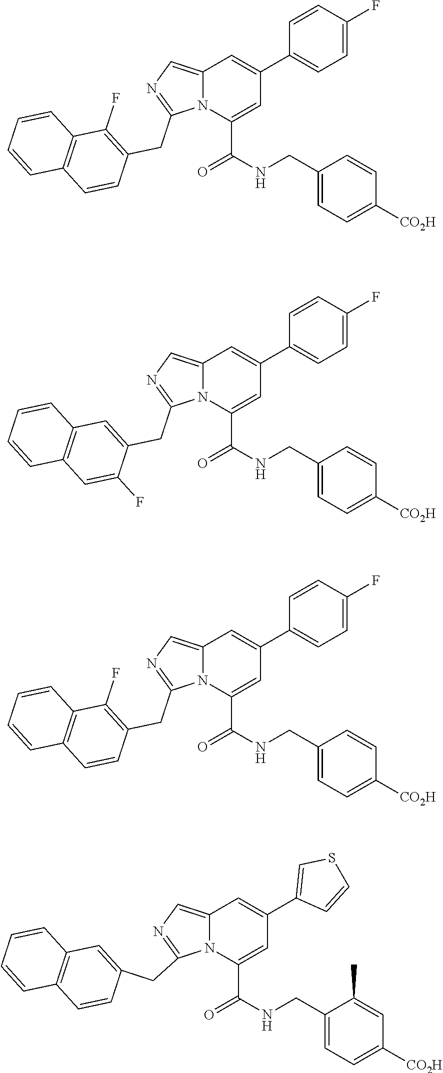

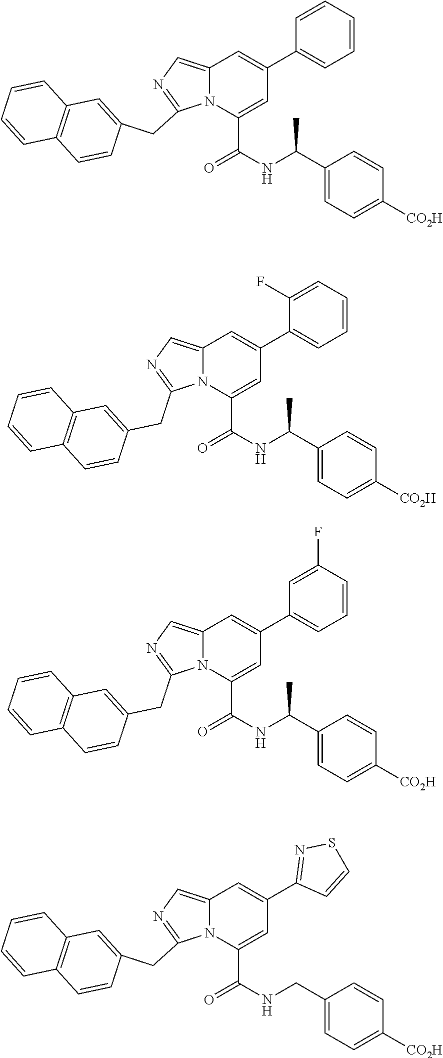

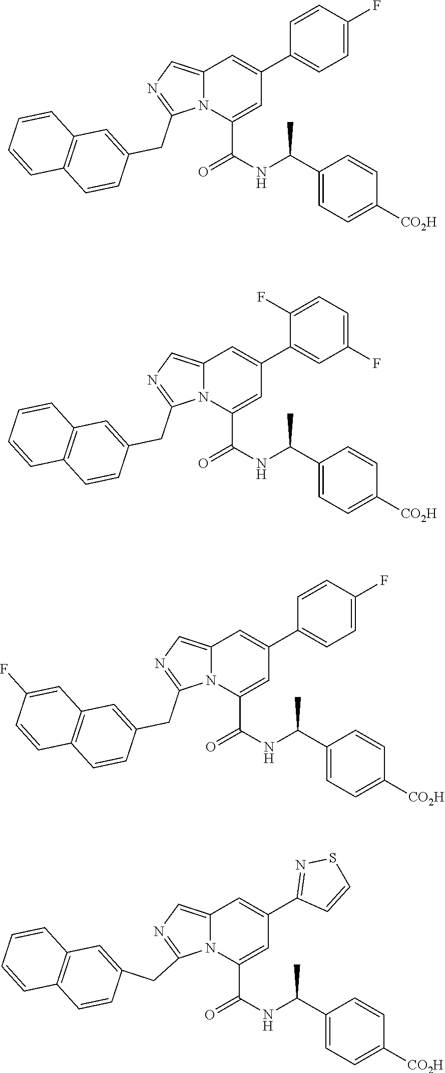 [SMILES: C[C@H](NC(=O)c1cc(-c2ccc(F)cc2)cc2cnc(Cc3ccc4ccccc4c3)n12)c1ccc(C(=O)O)cc1 ; C[C@H](NC(=O)c1cc(-c2cc(F)ccc2F)cc2cnc(Cc3ccc4ccccc4c3)n12)c1ccc(C(=O)O)cc1 ; C[C@H](NC(=O)c1cc(-c2ccc(F)cc2)cc2cnc(Cc3ccc4ccc(F)cc4c3)n12)c1ccc(C(=O)O)cc1 ; C[C@H](NC(=O)c1cc(-c2ccsn2)cc2cnc(Cc3ccc4ccccc4c3)n12)c1ccc(C(=O)O)cc1]

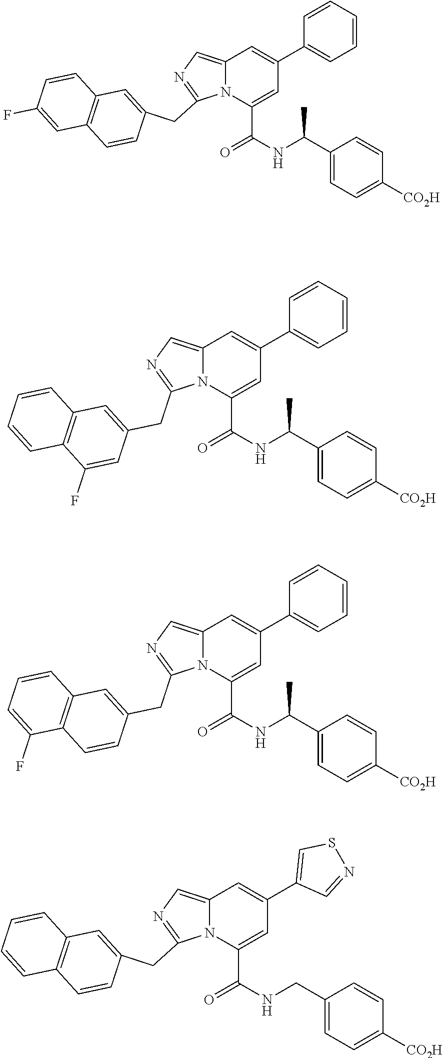 [SMILES: C[C@H](NC(=O)c1cc(-c2ccccc2)cc2cnc(Cc3ccc4cc(F)ccc4c3)n12)c1ccc(C(=O)O)cc1 ; C[C@H](NC(=O)c1cc(-c2ccccc2)cc2cnc(Cc3cc(F)c4ccccc4c3)n12)c1ccc(C(=O)O)cc1 ; C[C@H](NC(=O)c1cc(-c2ccccc2)cc2cnc(Cc3ccc4c(F)cccc4c3)n12)c1ccc(C(=O)O)cc1 ; O=C(O)c1ccc(CNC(=O)c2cc(-c3cnsc3)cc3cnc(Cc4ccc5ccccc5c4)n23)cc1]

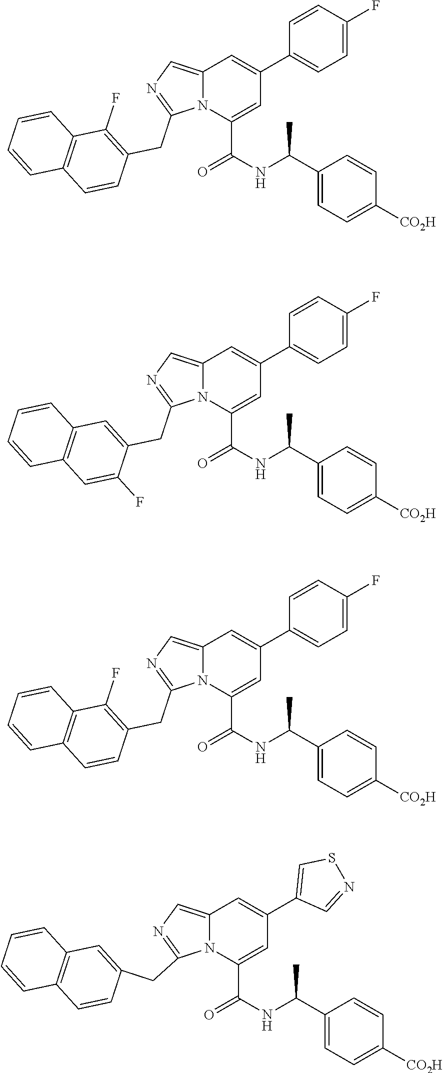 [SMILES: C[C@H](NC(=O)c1cc(-c2ccc(F)cc2)cc2cnc(Cc3ccc4ccccc4c3F)n12)c1ccc(C(=O)O)cc1 ; C[C@H](NC(=O)c1cc(-c2ccc(F)cc2)cc2cnc(Cc3cc4ccccc4cc3F)n12)c1ccc(C(=O)O)cc1 ; C[C@H](NC(=O)c1cc(-c2ccc(F)cc2)cc2cnc(Cc3ccc4ccccc4c3F)n12)c1ccc(C(=O)O)cc1 ; C[C@H](NC(=O)c1cc(-c2cnsc2)cc2cnc(Cc3ccc4ccccc4c3)n12)c1ccc(C(=O)O)cc1]

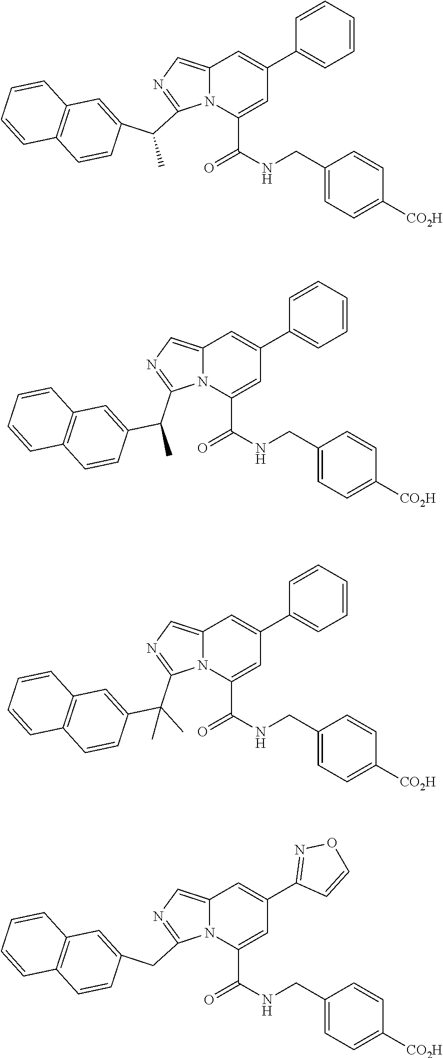 [SMILES: C[C@H](c1ccc2ccccc2c1)c1ncc2cc(-c3ccccc3)cc(C(=O)NCc3ccc(C(=O)O)cc3)n12 ; C[C@@H](c1ccc2ccccc2c1)c1ncc2cc(-c3ccccc3)cc(C(=O)NCc3ccc(C(=O)O)cc3)n12 ; CC(C)(c1ccc2ccccc2c1)c1ncc2cc(-c3ccccc3)cc(C(=O)NCc3ccc(C(=O)O)cc3)n12 ; O=C(O)c1ccc(CNC(=O)c2cc(-c3ccon3)cc3cnc(Cc4ccc5ccccc5c4)n23)cc1]

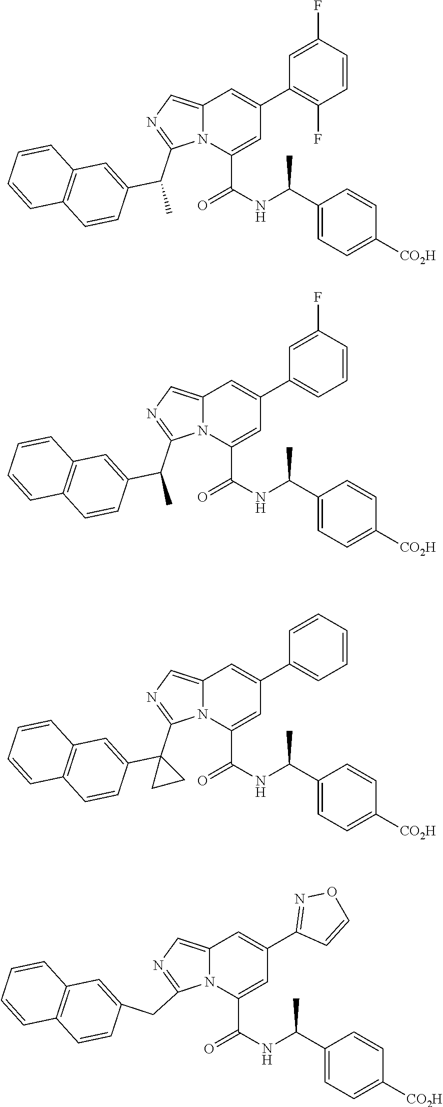 [SMILES: C[C@H](c1ccc2ccccc2c1)c1ncc2cc(-c3cc(F)ccc3F)cc(C(=O)N[C@@H](C)c3ccc(C(=O)O)cc3)n12 ; C[C@@H](c1ccc2ccccc2c1)c1ncc2cc(-c3cccc(F)c3)cc(C(=O)N[C@@H](C)c3ccc(C(=O)O)cc3)n12 ; C[C@H](NC(=O)c1cc(-c2ccccc2)cc2cnc(C3(c4ccc5ccccc5c4)CC3)n12)c1ccc(C(=O)O)cc1 ; C[C@H](NC(=O)c1cc(-c2ccon2)cc2cnc(Cc3ccc4ccccc4c3)n12)c1ccc(C(=O)O)cc1]

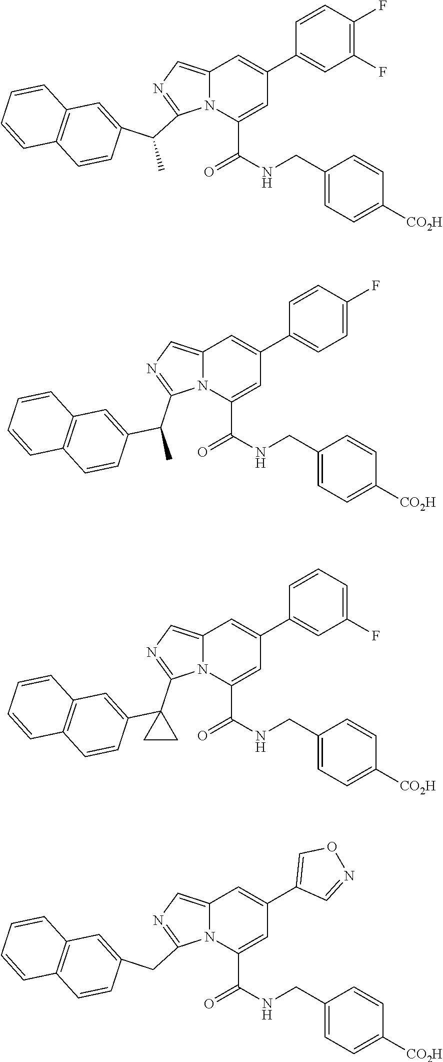 [SMILES: C[C@H](c1ccc2ccccc2c1)c1ncc2cc(-c3ccc(F)c(F)c3)cc(C(=O)NCc3ccc(C(=O)O)cc3)n12 ; C[C@@H](c1ccc2ccccc2c1)c1ncc2cc(-c3ccc(F)cc3)cc(C(=O)NCc3ccc(C(=O)O)cc3)n12 ; O=C(O)c1ccc(CNC(=O)c2cc(-c3cccc(F)c3)cc3cnc(C4(c5ccc6ccccc6c5)CC4)n23)cc1 ; O=C(O)c1ccc(CNC(=O)c2cc(-c3cnoc3)cc3cnc(Cc4ccc5ccccc5c4)n23)cc1]

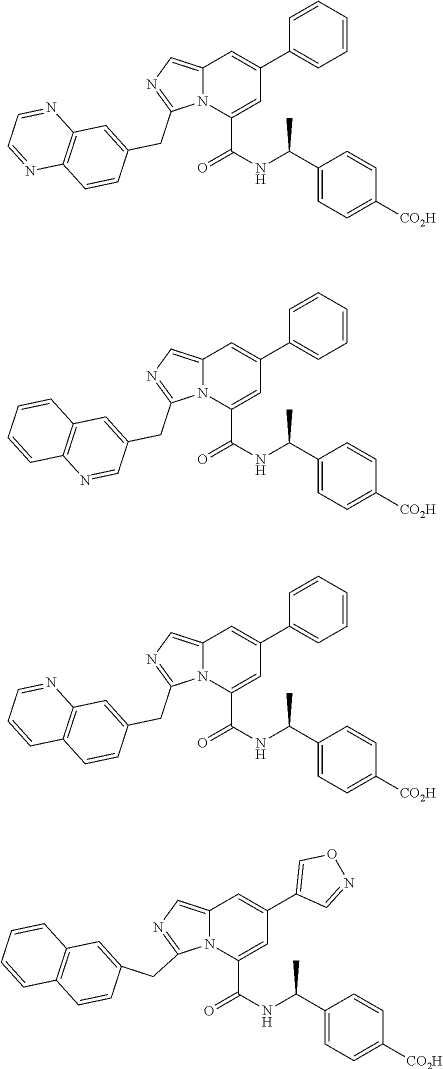 [SMILES: C[C@H](NC(=O)c1cc(-c2ccccc2)cc2cnc(Cc3ccc4nccnc4c3)n12)c1ccc(C(=O)O)cc1 ; C[C@H](NC(=O)c1cc(-c2ccccc2)cc2cnc(Cc3cnc4ccccc4c3)n12)c1ccc(C(=O)O)cc1 ; C[C@H](NC(=O)c1cc(-c2ccccc2)cc2cnc(Cc3ccc4cccnc4c3)n12)c1ccc(C(=O)O)cc1 ; C[C@H](NC(=O)c1cc(-c2cnoc2)cc2cnc(Cc3ccc4ccccc4c3)n12)c1ccc(C(=O)O)cc1]

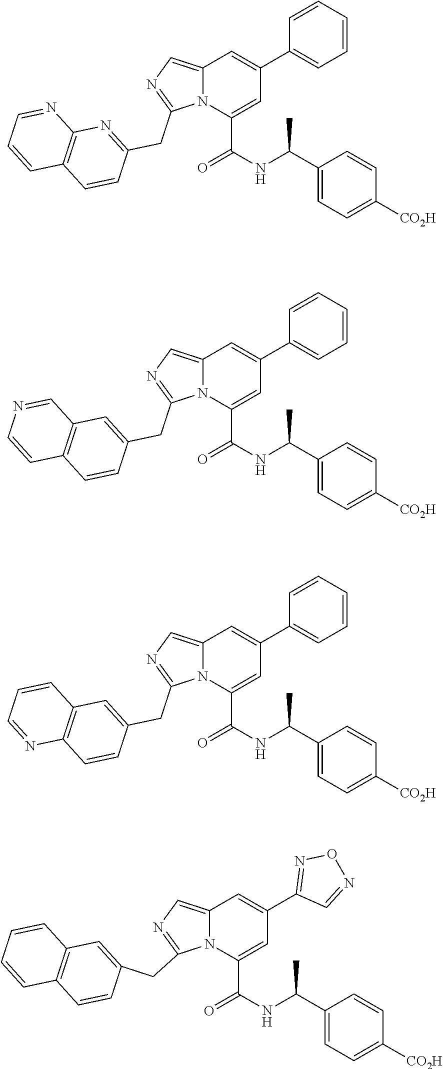 [SMILES: C[C@H](NC(=O)c1cc(-c2ccccc2)cc2cnc(Cc3ccc4cccnc4n3)n12)c1ccc(C(=O)O)cc1 ; C[C@H](NC(=O)c1cc(-c2ccccc2)cc2cnc(Cc3ccc4ccncc4c3)n12)c1ccc(C(=O)O)cc1 ; C[C@H](NC(=O)c1cc(-c2ccccc2)cc2cnc(Cc3ccc4ncccc4c3)n12)c1ccc(C(=O)O)cc1 ; C[C@H](NC(=O)c1cc(-c2cnon2)cc2cnc(Cc3ccc4ccccc4c3)n12)c1ccc(C(=O)O)cc1]

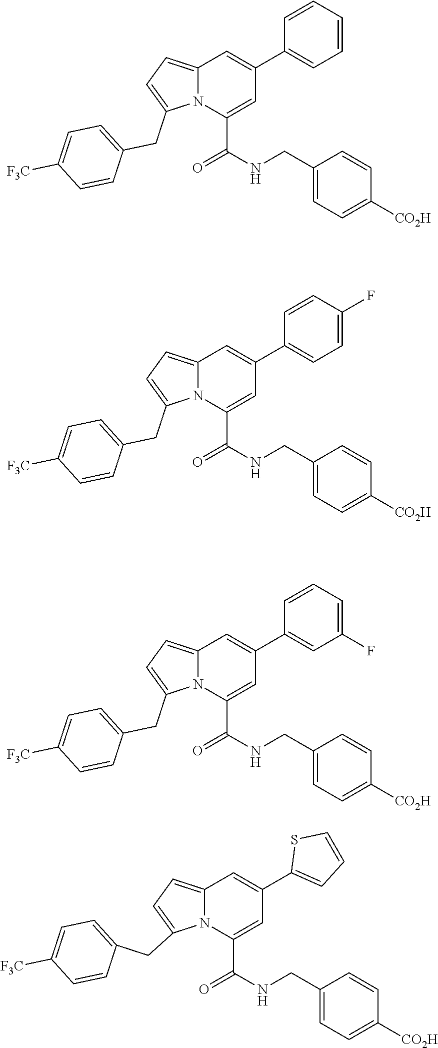

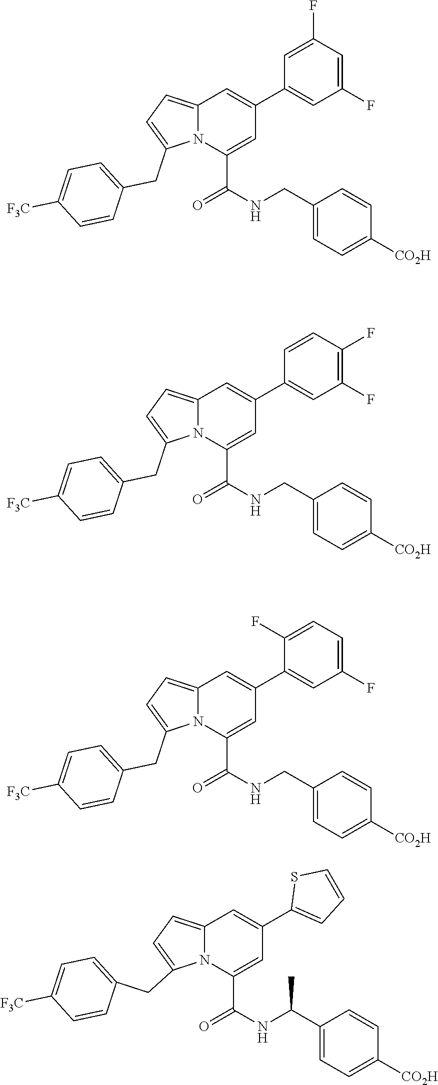

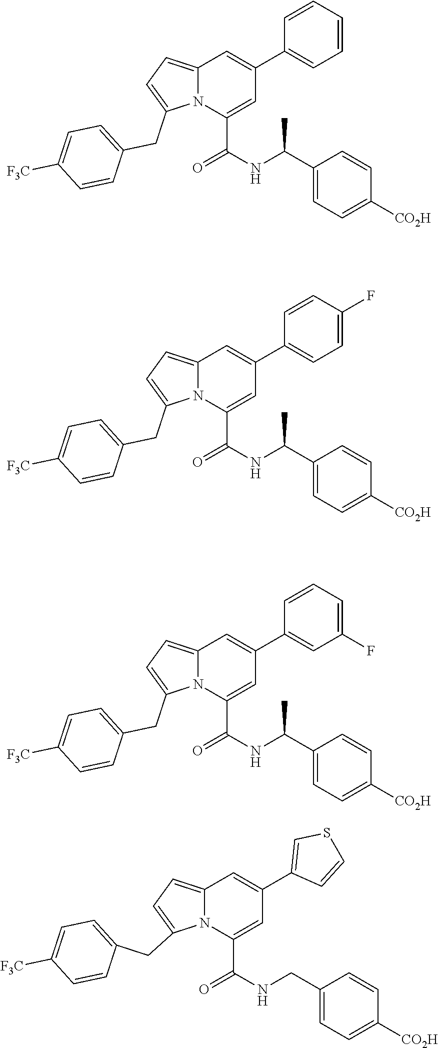

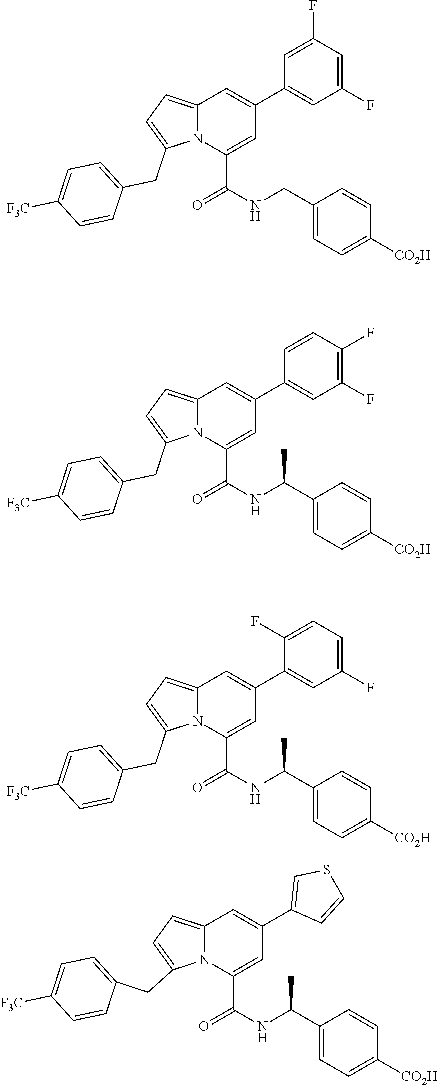

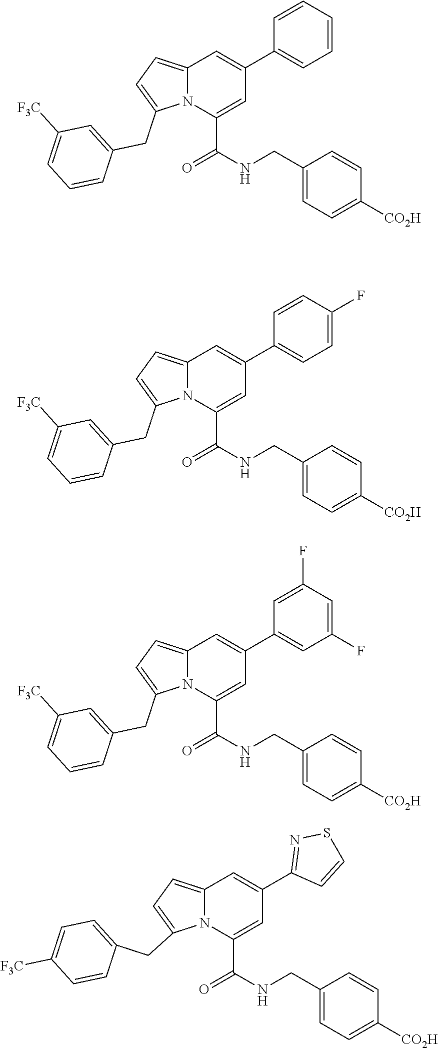

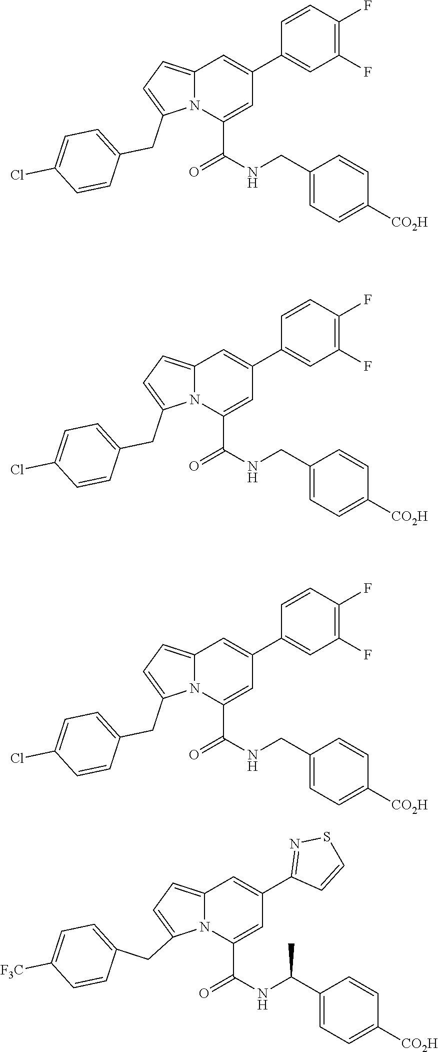

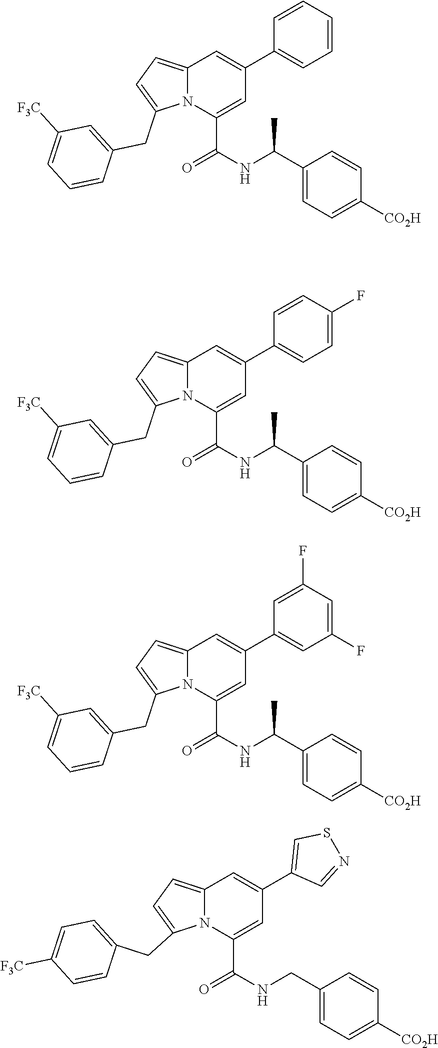

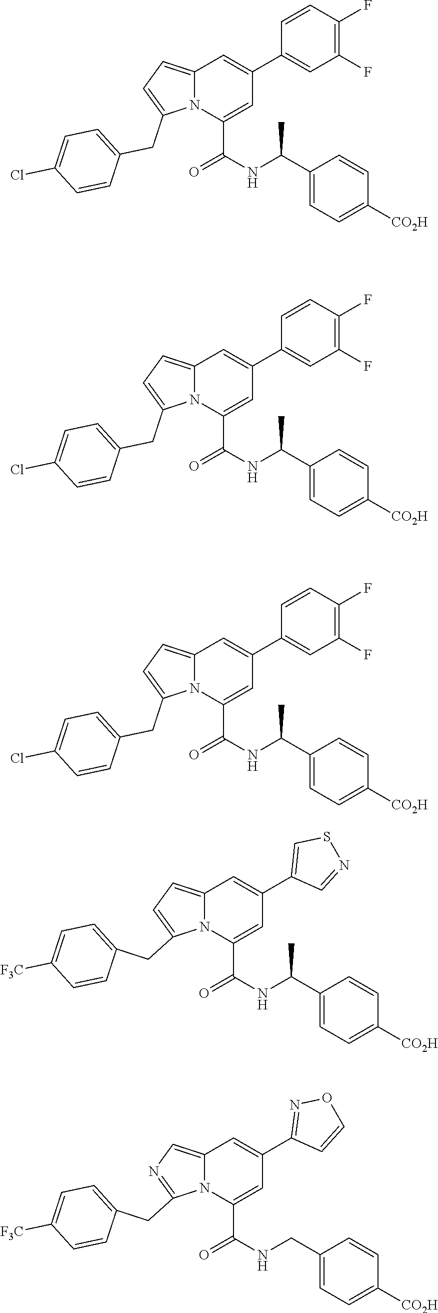

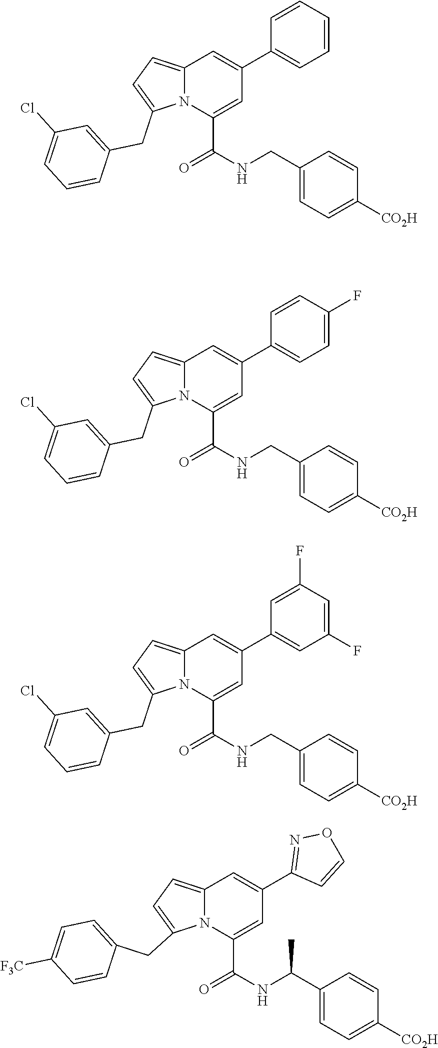

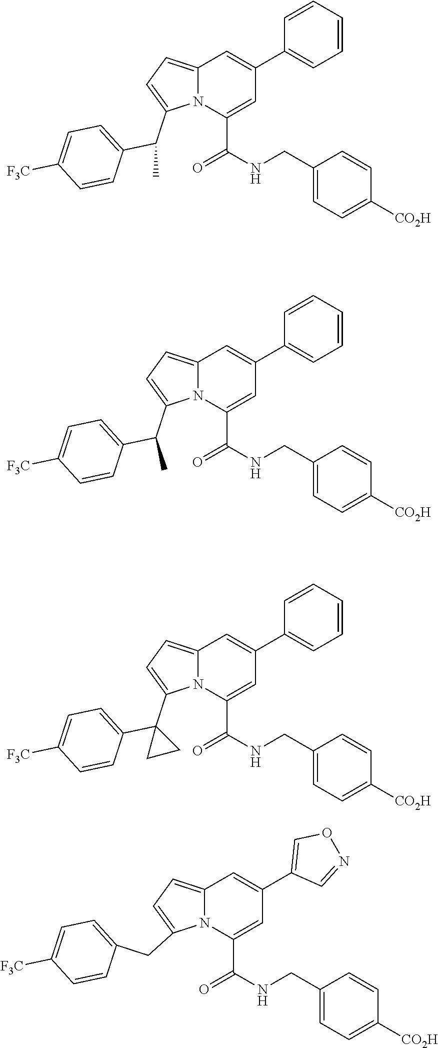

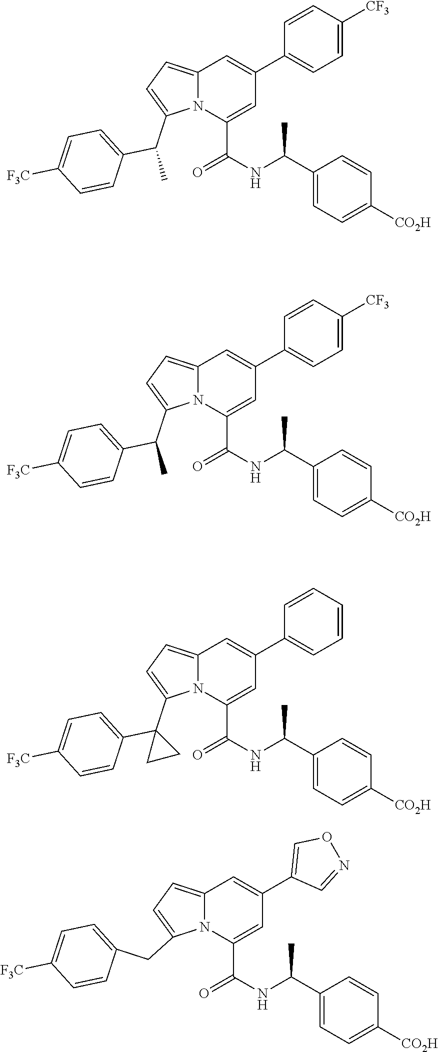

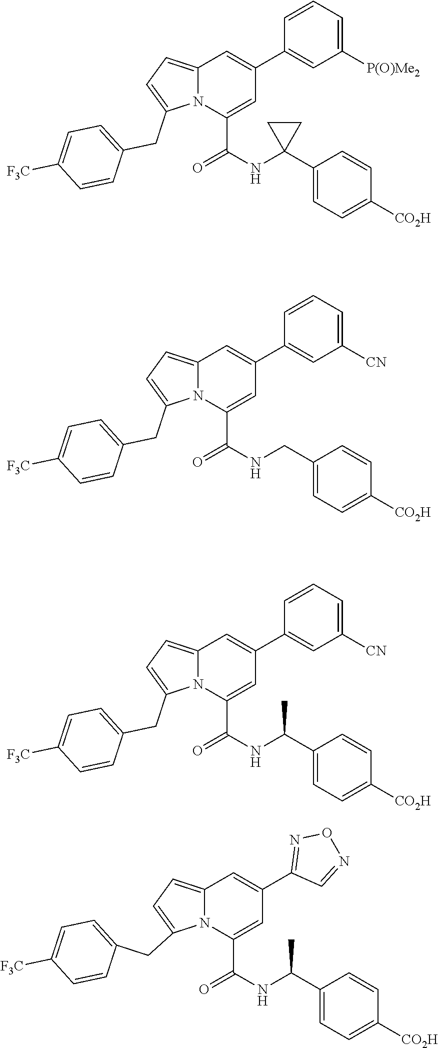

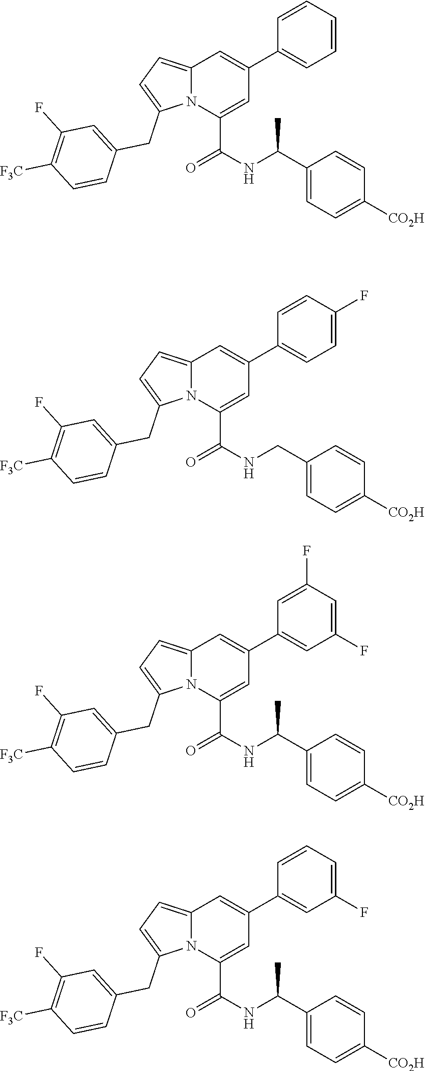 [SMILES: C[C@H](NC(=O)c1cc(-c2ccccc2)cc2ccc(Cc3ccc(C(F)(F)F)c(F)c3)n12)c1ccc(C(=O)O)cc1 ; O=C(O)c1ccc(CNC(=O)c2cc(-c3ccc(F)cc3)cc3ccc(Cc4ccc(C(F)(F)F)c(F)c4)n23)cc1 ; C[C@H](NC(=O)c1cc(-c2cc(F)cc(F)c2)cc2ccc(Cc3ccc(C(F)(F)F)c(F)c3)n12)c1ccc(C(=O)O)cc1 ; C[C@H](NC(=O)c1cc(-c2cccc(F)c2)cc2ccc(Cc3ccc(C(F)(F)F)c(F)c3)n12)c1ccc(C(=O)O)cc1]

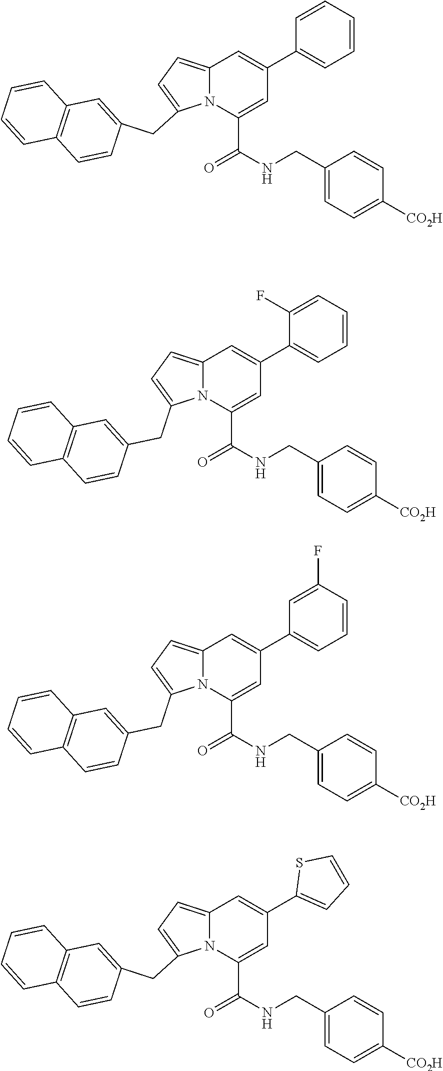 [SMILES: O=C(O)c1ccc(CNC(=O)c2cc(-c3ccccc3)cc3ccc(Cc4ccc5ccccc5c4)n23)cc1 ; O=C(O)c1ccc(CNC(=O)c2cc(-c3ccccc3F)cc3ccc(Cc4ccc5ccccc5c4)n23)cc1 ; O=C(O)c1ccc(CNC(=O)c2cc(-c3cccc(F)c3)cc3ccc(Cc4ccc5ccccc5c4)n23)cc1 ; O=C(O)c1ccc(CNC(=O)c2cc(-c3cccs3)cc3ccc(Cc4ccc5ccccc5c4)n23)cc1]

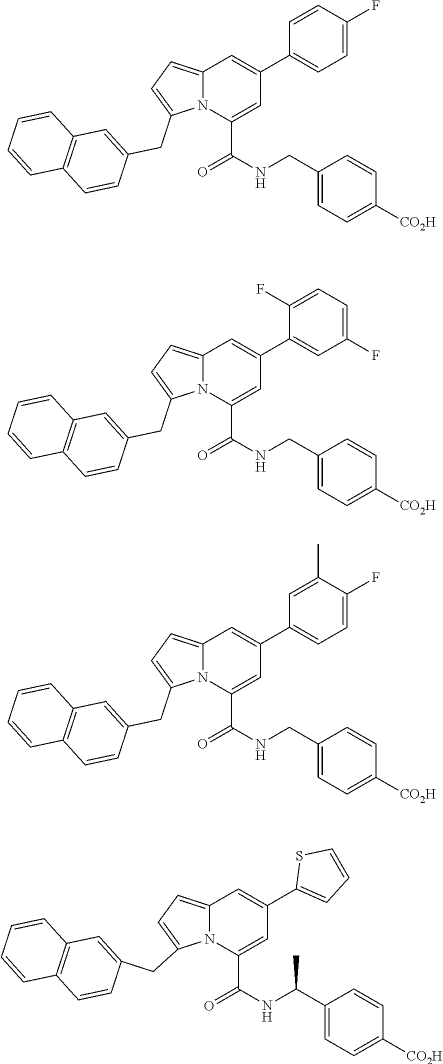 [SMILES: O=C(O)c1ccc(CNC(=O)c2cc(-c3ccc(F)cc3)cc3ccc(Cc4ccc5ccccc5c4)n23)cc1 ; O=C(O)c1ccc(CNC(=O)c2cc(-c3cc(F)ccc3F)cc3ccc(Cc4ccc5ccccc5c4)n23)cc1 ; Cc1cc(-c2cc(C(=O)NCc3ccc(C(=O)O)cc3)n3c(Cc4ccc5ccccc5c4)ccc3c2)ccc1F ; C[C@H](NC(=O)c1cc(-c2cccs2)cc2ccc(Cc3ccc4ccccc4c3)n12)c1ccc(C(=O)O)cc1]

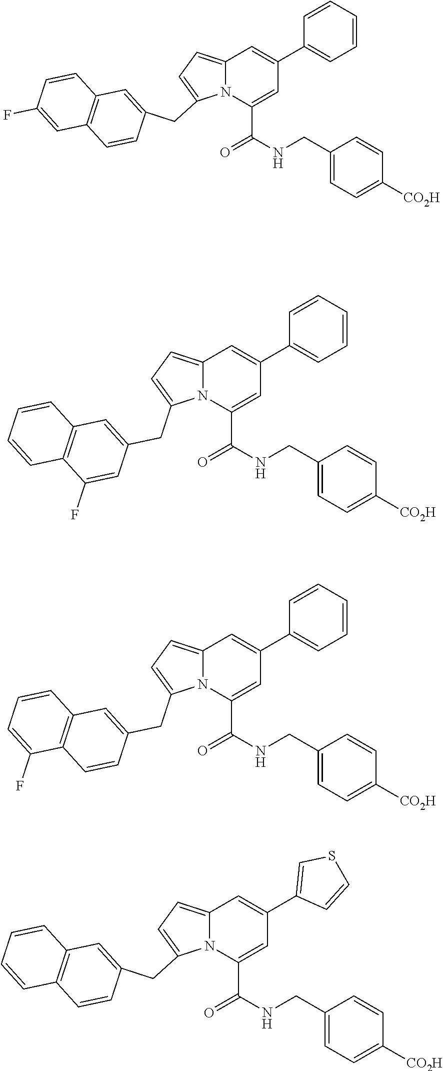 [SMILES: O=C(O)c1ccc(CNC(=O)c2cc(-c3ccccc3)cc3ccc(Cc4ccc5cc(F)ccc5c4)n23)cc1 ; O=C(O)c1ccc(CNC(=O)c2cc(-c3ccccc3)cc3ccc(Cc4cc(F)c5ccccc5c4)n23)cc1 ; O=C(O)c1ccc(CNC(=O)c2cc(-c3ccccc3)cc3ccc(Cc4ccc5c(F)cccc5c4)n23)cc1 ; O=C(O)c1ccc(CNC(=O)c2cc(-c3ccsc3)cc3ccc(Cc4ccc5ccccc5c4)n23)cc1]

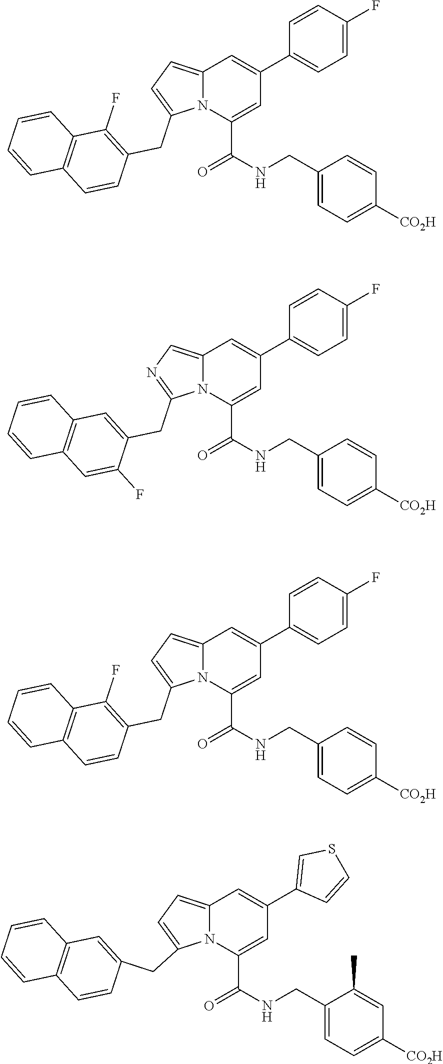

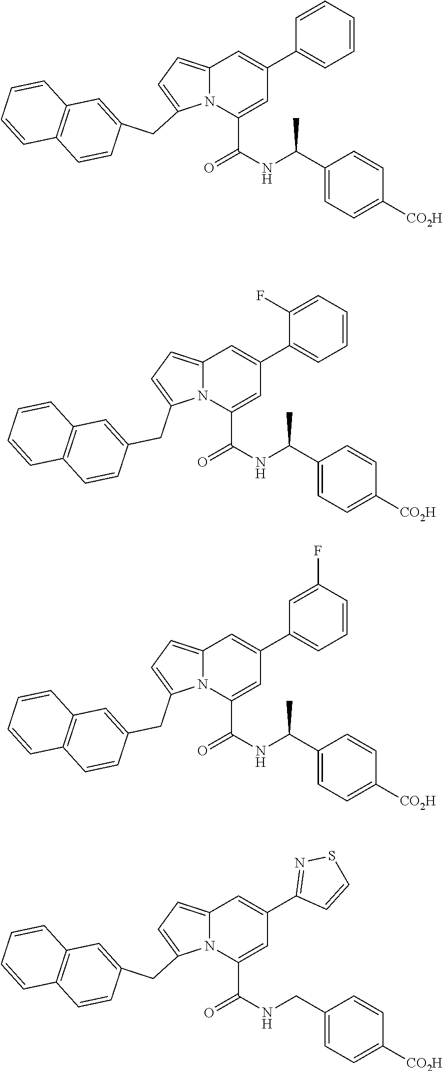 [SMILES: C[C@H](NC(=O)c1cc(-c2ccccc2)cc2ccc(Cc3ccc4ccccc4c3)n12)c1ccc(C(=O)O)cc1 ; C[C@H](NC(=O)c1cc(-c2ccccc2F)cc2ccc(Cc3ccc4ccccc4c3)n12)c1ccc(C(=O)O)cc1 ; C[C@H](NC(=O)c1cc(-c2cccc(F)c2)cc2ccc(Cc3ccc4ccccc4c3)n12)c1ccc(C(=O)O)cc1 ; O=C(O)c1ccc(CNC(=O)c2cc(-c3ccsn3)cc3ccc(Cc4ccc5ccccc5c4)n23)cc1]

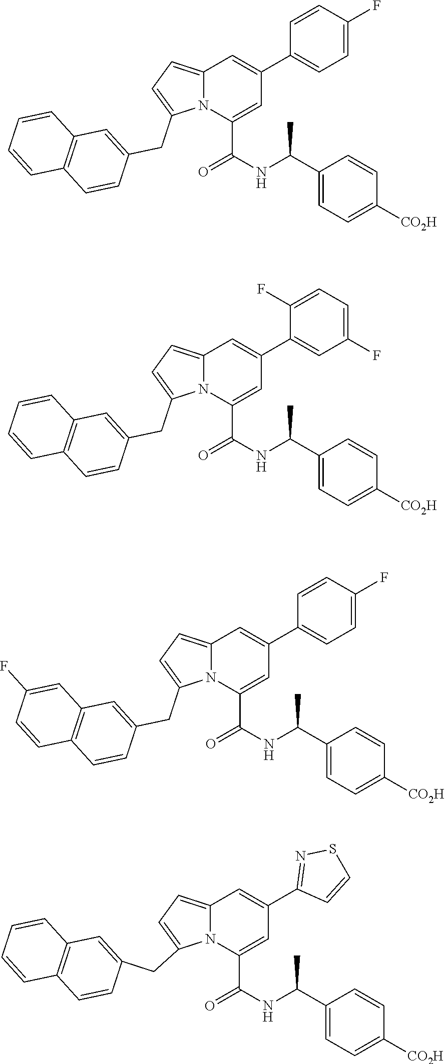 [SMILES: C[C@H](NC(=O)c1cc(-c2ccc(F)cc2)cc2ccc(Cc3ccc4ccccc4c3)n12)c1ccc(C(=O)O)cc1 ; C[C@H](NC(=O)c1cc(-c2cc(F)ccc2F)cc2ccc(Cc3ccc4ccccc4c3)n12)c1ccc(C(=O)O)cc1 ; C[C@H](NC(=O)c1cc(-c2ccc(F)cc2)cc2ccc(Cc3ccc4ccc(F)cc4c3)n12)c1ccc(C(=O)O)cc1 ; C[C@H](NC(=O)c1cc(-c2ccsn2)cc2ccc(Cc3ccc4ccccc4c3)n12)c1ccc(C(=O)O)cc1]

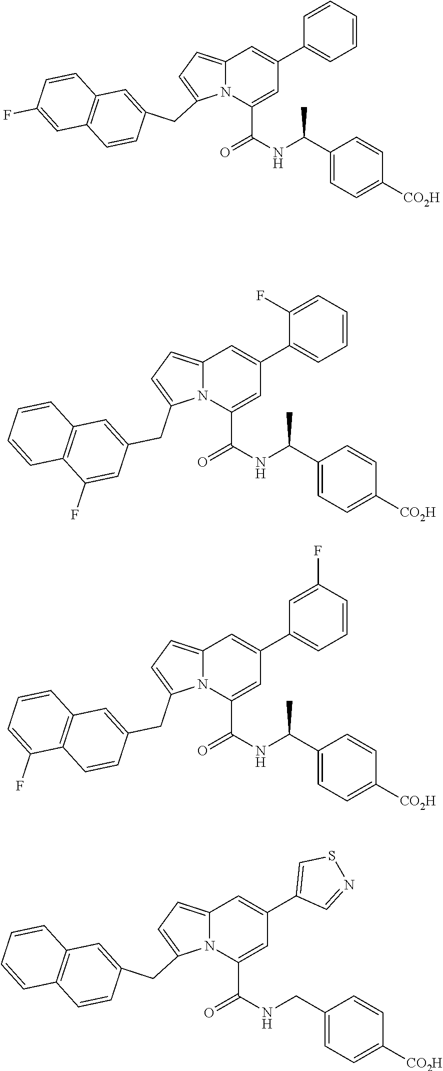 [SMILES: C[C@H](NC(=O)c1cc(-c2ccccc2)cc2ccc(Cc3ccc4cc(F)ccc4c3)n12)c1ccc(C(=O)O)cc1 ; C[C@H](NC(=O)c1cc(-c2ccccc2F)cc2ccc(Cc3cc(F)c4ccccc4c3)n12)c1ccc(C(=O)O)cc1 ; C[C@H](NC(=O)c1cc(-c2cccc(F)c2)cc2ccc(Cc3ccc4c(F)cccc4c3)n12)c1ccc(C(=O)O)cc1 ; O=C(O)c1ccc(CNC(=O)c2cc(-c3cnsc3)cc3ccc(Cc4ccc5ccccc5c4)n23)cc1]

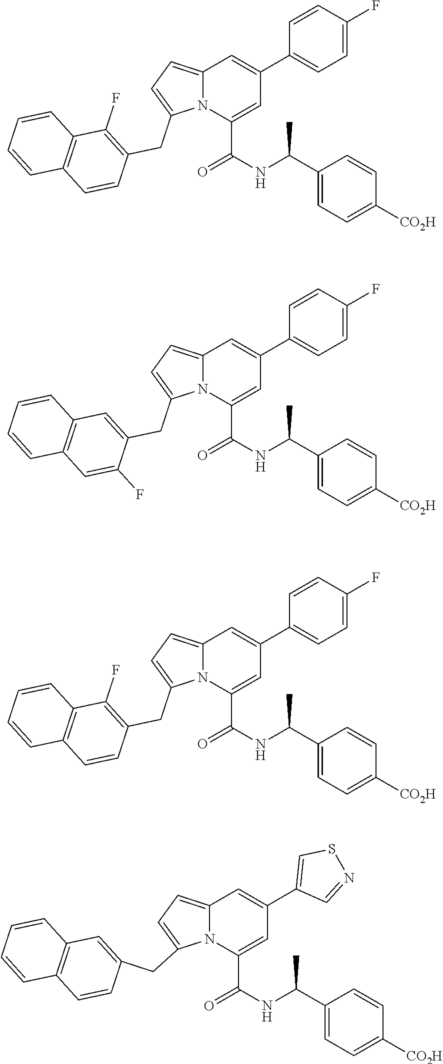 [SMILES: C[C@H](NC(=O)c1cc(-c2ccc(F)cc2)cc2ccc(Cc3ccc4ccccc4c3F)n12)c1ccc(C(=O)O)cc1 ; C[C@H](NC(=O)c1cc(-c2ccc(F)cc2)cc2ccc(Cc3cc4ccccc4cc3F)n12)c1ccc(C(=O)O)cc1 ; C[C@H](NC(=O)c1cc(-c2ccc(F)cc2)cc2ccc(Cc3ccc4ccccc4c3F)n12)c1ccc(C(=O)O)cc1 ; C[C@H](NC(=O)c1cc(-c2cnsc2)cc2ccc(Cc3ccc4ccccc4c3)n12)c1ccc(C(=O)O)cc1]

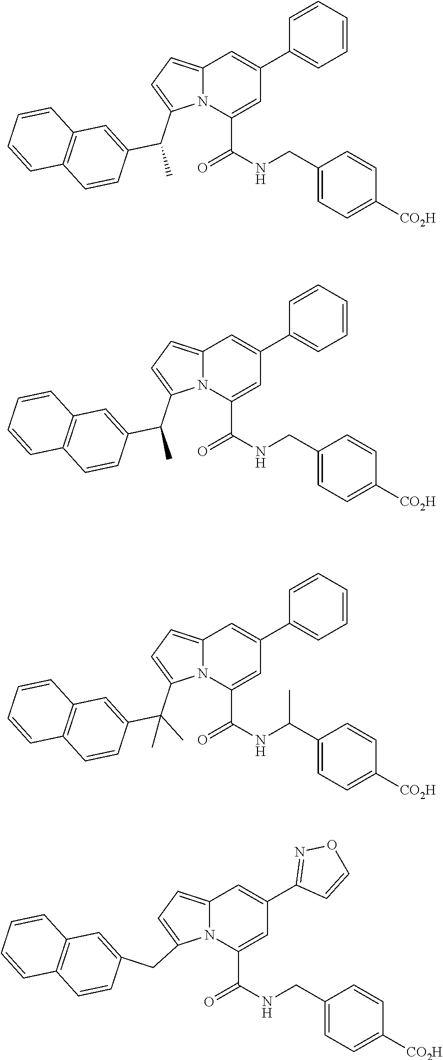 [SMILES: C[C@H](c1ccc2ccccc2c1)c1ccc2cc(-c3ccccc3)cc(C(=O)NCc3ccc(C(=O)O)cc3)n12 ; C[C@@H](c1ccc2ccccc2c1)c1ccc2cc(-c3ccccc3)cc(C(=O)NCc3ccc(C(=O)O)cc3)n12 ; CC(NC(=O)c1cc(-c2ccccc2)cc2ccc(C(C)(C)c3ccc4ccccc4c3)n12)c1ccc(C(=O)O)cc1 ; O=C(O)c1ccc(CNC(=O)c2cc(-c3ccon3)cc3ccc(Cc4ccc5ccccc5c4)n23)cc1]

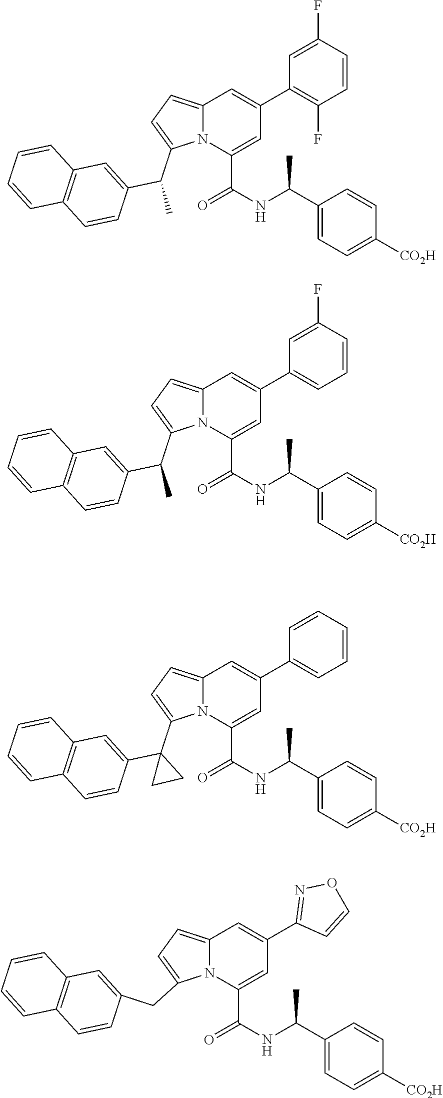

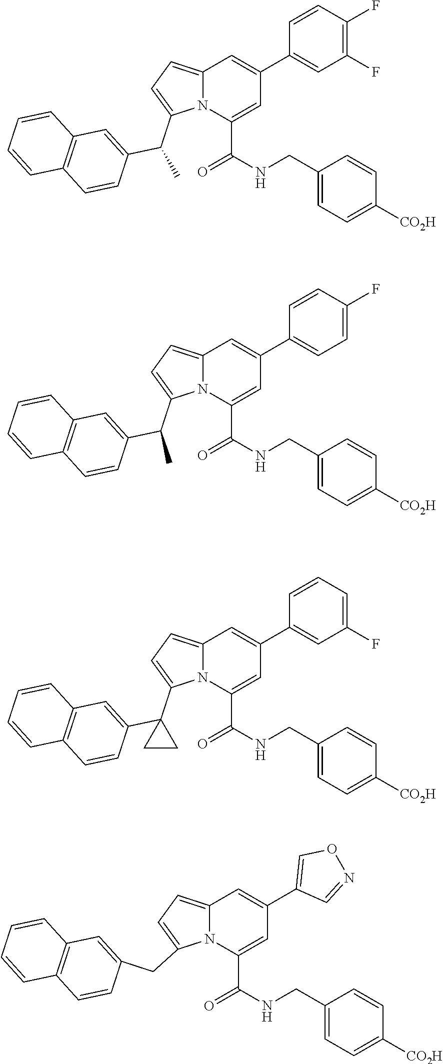 [SMILES: C[C@H](c1ccc2ccccc2c1)c1ccc2cc(-c3ccc(F)c(F)c3)cc(C(=O)NCc3ccc(C(=O)O)cc3)n12 ; C[C@@H](c1ccc2ccccc2c1)c1ccc2cc(-c3ccc(F)cc3)cc(C(=O)NCc3ccc(C(=O)O)cc3)n12 ; O=C(O)c1ccc(CNC(=O)c2cc(-c3cccc(F)c3)cc3ccc(C4(c5ccc6ccccc6c5)CC4)n23)cc1 ; O=C(O)c1ccc(CNC(=O)c2cc(-c3cnoc3)cc3ccc(Cc4ccc5ccccc5c4)n23)cc1]

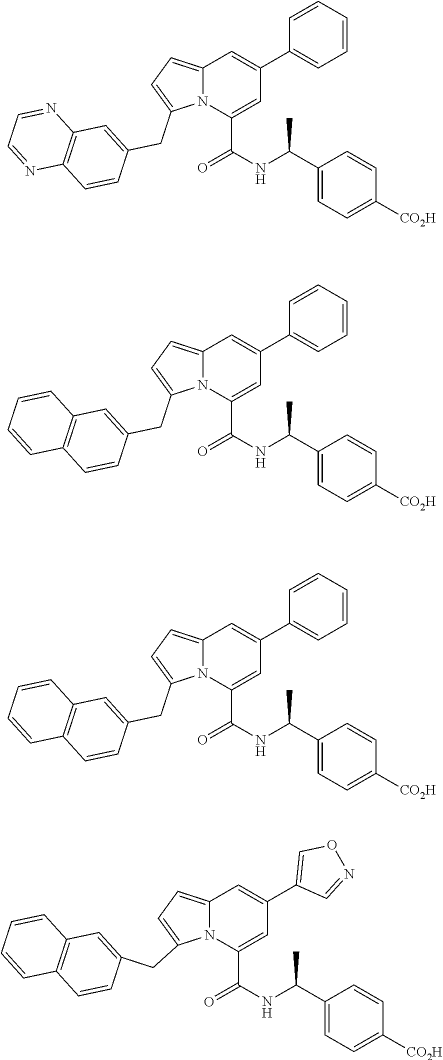 [SMILES: C[C@H](NC(=O)c1cc(-c2ccccc2)cc2ccc(Cc3ccc4nccnc4c3)n12)c1ccc(C(=O)O)cc1 ; C[C@H](NC(=O)c1cc(-c2ccccc2)cc2ccc(Cc3ccc4ccccc4c3)n12)c1ccc(C(=O)O)cc1 ; C[C@H](NC(=O)c1cc(-c2ccccc2)cc2ccc(Cc3ccc4ccccc4c3)n12)c1ccc(C(=O)O)cc1 ; C[C@H](NC(=O)c1cc(-c2cnoc2)cc2ccc(Cc3ccc4ccccc4c3)n12)c1ccc(C(=O)O)cc1]

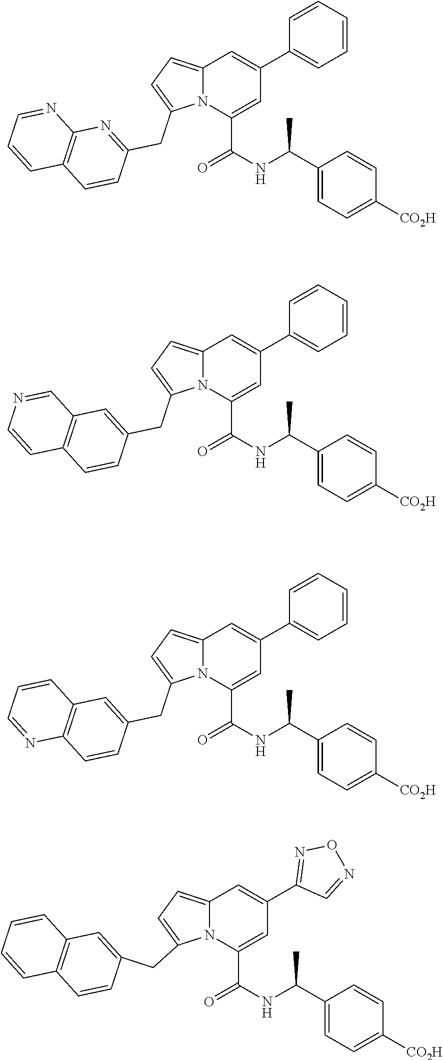 [SMILES: C[C@H](NC(=O)c1cc(-c2ccccc2)cc2ccc(Cc3ccc4cccnc4n3)n12)c1ccc(C(=O)O)cc1 ; C[C@H](NC(=O)c1cc(-c2ccccc2)cc2ccc(Cc3ccc4ccncc4c3)n12)c1ccc(C(=O)O)cc1 ; C[C@H](NC(=O)c1cc(-c2ccccc2)cc2ccc(Cc3ccc4ncccc4c3)n12)c1ccc(C(=O)O)cc1 ; C[C@H](NC(=O)c1cc(-c2cnon2)cc2ccc(Cc3ccc4ccccc4c3)n12)c1ccc(C(=O)O)cc1]

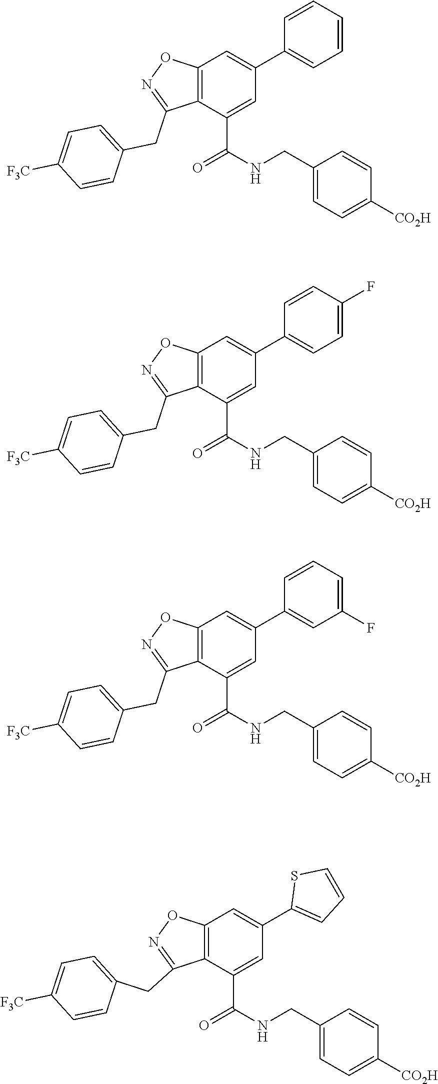 [SMILES: O=C(O)c1ccc(CNC(=O)c2cc(-c3ccccc3)cc3onc(Cc4ccc(C(F)(F)F)cc4)c23)cc1 ; O=C(O)c1ccc(CNC(=O)c2cc(-c3ccc(F)cc3)cc3onc(Cc4ccc(C(F)(F)F)cc4)c23)cc1 ; O=C(O)c1ccc(CNC(=O)c2cc(-c3cccc(F)c3)cc3onc(Cc4ccc(C(F)(F)F)cc4)c23)cc1 ; O=C(O)c1ccc(CNC(=O)c2cc(-c3cccs3)cc3onc(Cc4ccc(C(F)(F)F)cc4)c23)cc1]

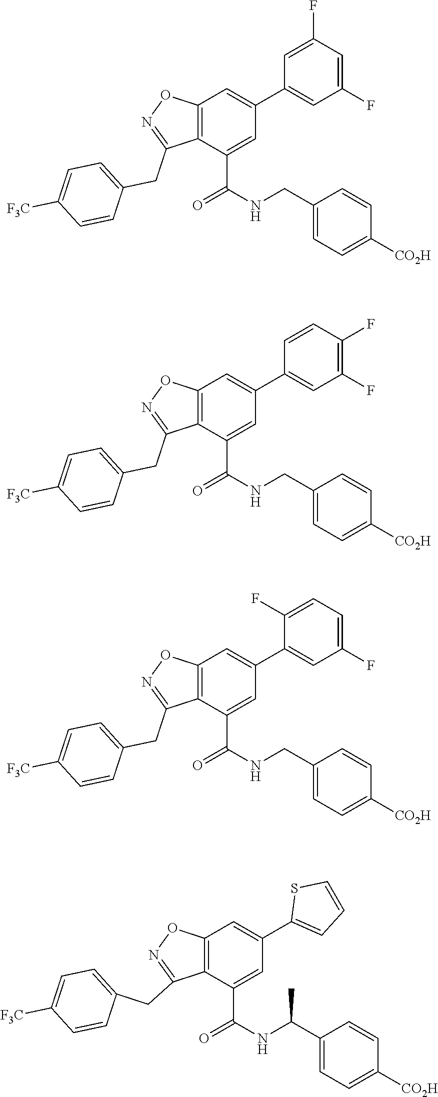 [SMILES: O=C(O)c1ccc(CNC(=O)c2cc(-c3cc(F)cc(F)c3)cc3onc(Cc4ccc(C(F)(F)F)cc4)c23)cc1 ; O=C(O)c1ccc(CNC(=O)c2cc(-c3ccc(F)c(F)c3)cc3onc(Cc4ccc(C(F)(F)F)cc4)c23)cc1 ; O=C(O)c1ccc(CNC(=O)c2cc(-c3cc(F)ccc3F)cc3onc(Cc4ccc(C(F)(F)F)cc4)c23)cc1 ; C[C@H](NC(=O)c1cc(-c2cccs2)cc2onc(Cc3ccc(C(F)(F)F)cc3)c12)c1ccc(C(=O)O)cc1]

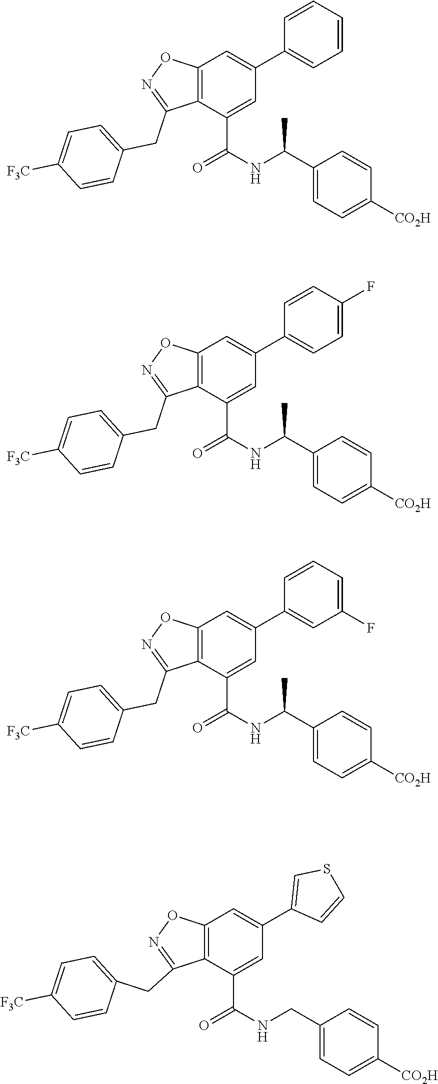 [SMILES: C[C@H](NC(=O)c1cc(-c2ccccc2)cc2onc(Cc3ccc(C(F)(F)F)cc3)c12)c1ccc(C(=O)O)cc1 ; C[C@H](NC(=O)c1cc(-c2ccc(F)cc2)cc2onc(Cc3ccc(C(F)(F)F)cc3)c12)c1ccc(C(=O)O)cc1 ; C[C@H](NC(=O)c1cc(-c2cccc(F)c2)cc2onc(Cc3ccc(C(F)(F)F)cc3)c12)c1ccc(C(=O)O)cc1 ; O=C(O)c1ccc(CNC(=O)c2cc(-c3ccsc3)cc3onc(Cc4ccc(C(F)(F)F)cc4)c23)cc1]

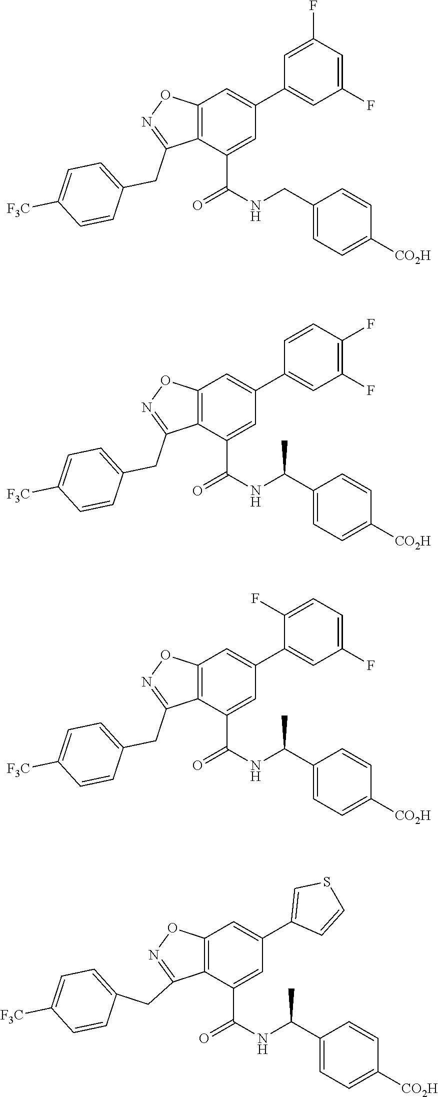 [SMILES: O=C(O)c1ccc(CNC(=O)c2cc(-c3cc(F)cc(F)c3)cc3onc(Cc4ccc(C(F)(F)F)cc4)c23)cc1 ; C[C@H](NC(=O)c1cc(-c2ccc(F)c(F)c2)cc2onc(Cc3ccc(C(F)(F)F)cc3)c12)c1ccc(C(=O)O)cc1 ; C[C@H](NC(=O)c1cc(-c2cc(F)ccc2F)cc2onc(Cc3ccc(C(F)(F)F)cc3)c12)c1ccc(C(=O)O)cc1 ; C[C@H](NC(=O)c1cc(-c2ccsc2)cc2onc(Cc3ccc(C(F)(F)F)cc3)c12)c1ccc(C(=O)O)cc1]

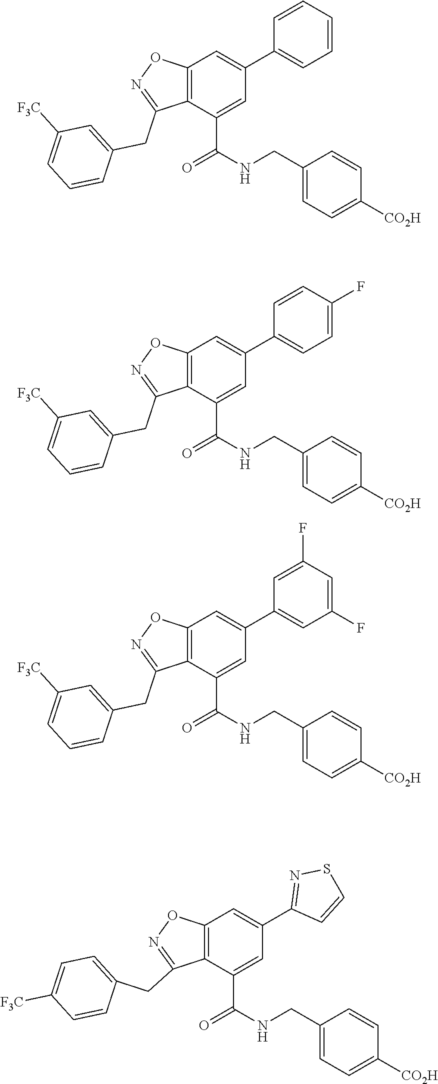

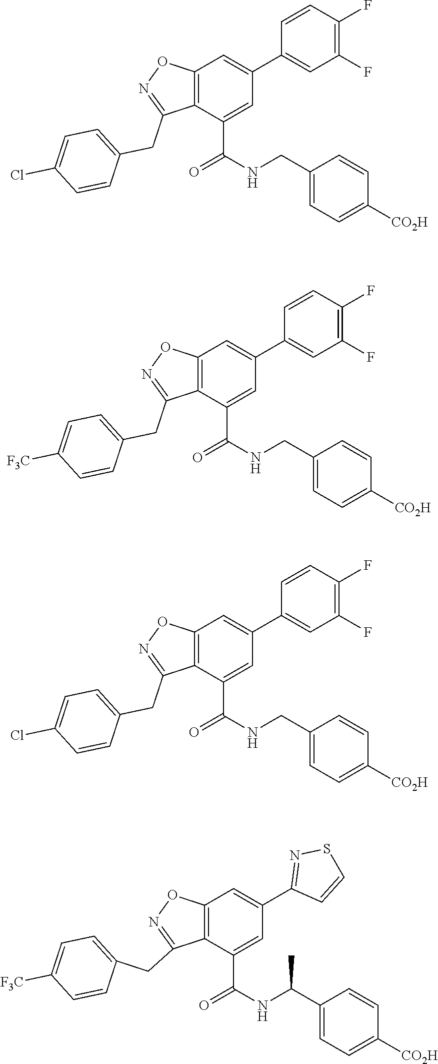 [SMILES: O=C(O)c1ccc(CNC(=O)c2cc(-c3ccc(F)c(F)c3)cc3onc(Cc4ccc(Cl)cc4)c23)cc1 ; O=C(O)c1ccc(CNC(=O)c2cc(-c3ccc(F)c(F)c3)cc3onc(Cc4ccc(C(F)(F)F)cc4)c23)cc1 ; O=C(O)c1ccc(CNC(=O)c2cc(-c3ccc(F)c(F)c3)cc3onc(Cc4ccc(Cl)cc4)c23)cc1 ; C[C@H](NC(=O)c1cc(-c2ccsn2)cc2onc(Cc3ccc(C(F)(F)F)cc3)c12)c1ccc(C(=O)O)cc1]

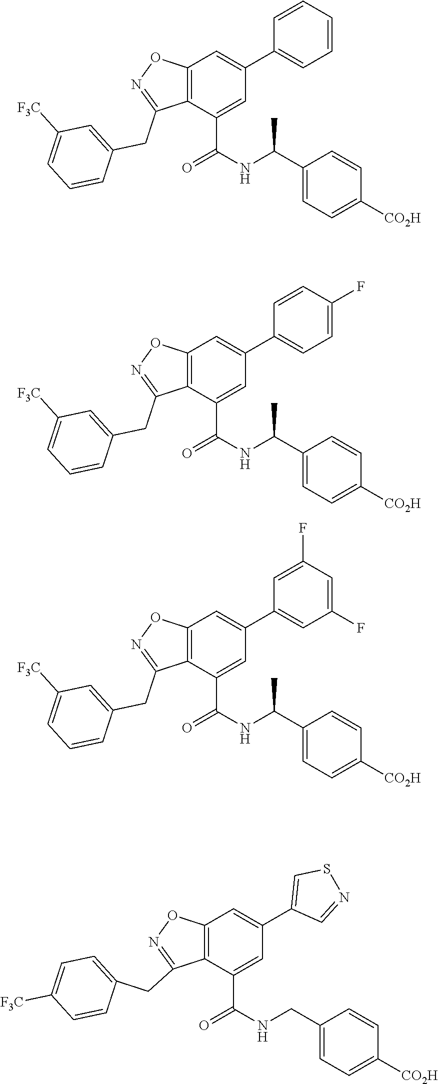

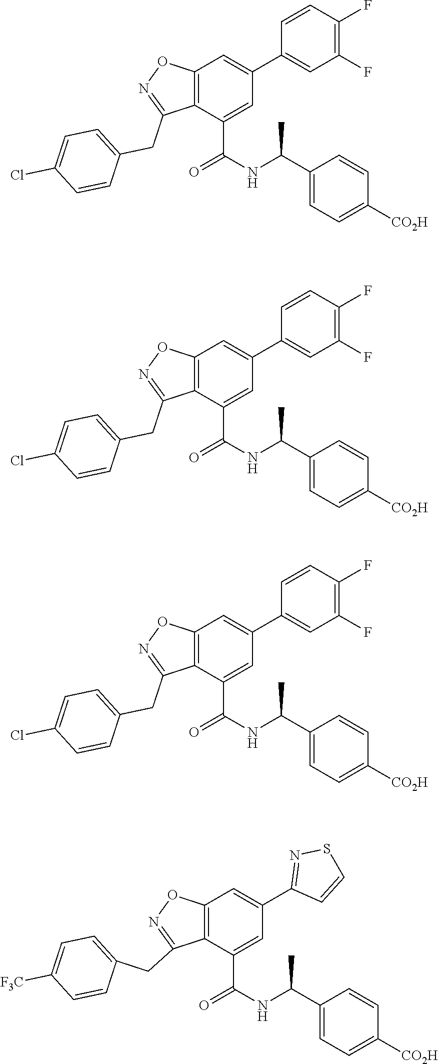 [SMILES: C[C@H](NC(=O)c1cc(-c2ccc(F)c(F)c2)cc2onc(Cc3ccc(Cl)cc3)c12)c1ccc(C(=O)O)cc1 ; C[C@H](NC(=O)c1cc(-c2ccc(F)c(F)c2)cc2onc(Cc3ccc(Cl)cc3)c12)c1ccc(C(=O)O)cc1 ; C[C@H](NC(=O)c1cc(-c2ccc(F)c(F)c2)cc2onc(Cc3ccc(Cl)cc3)c12)c1ccc(C(=O)O)cc1 ; C[C@H](NC(=O)c1cc(-c2ccsn2)cc2onc(Cc3ccc(C(F)(F)F)cc3)c12)c1ccc(C(=O)O)cc1]

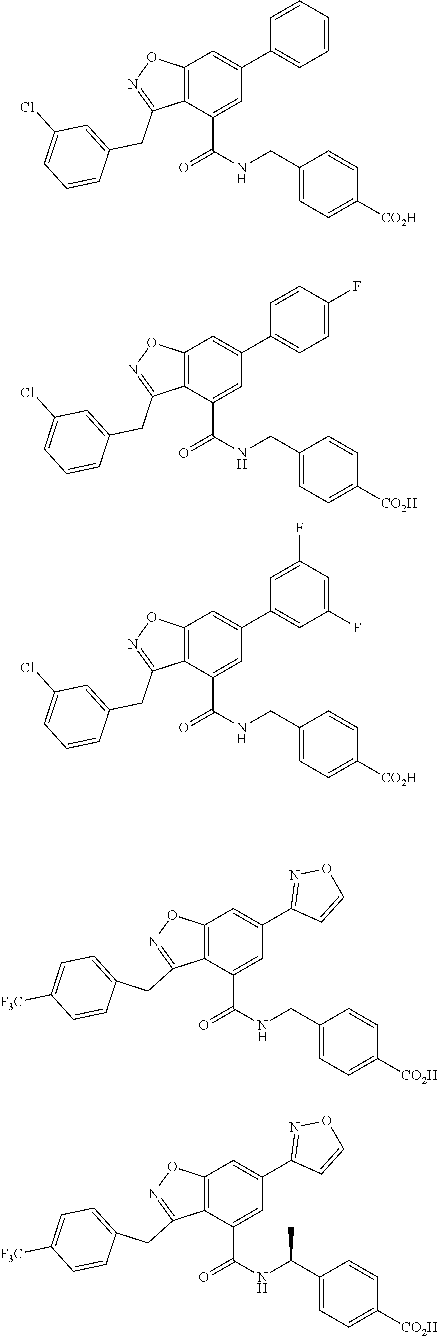

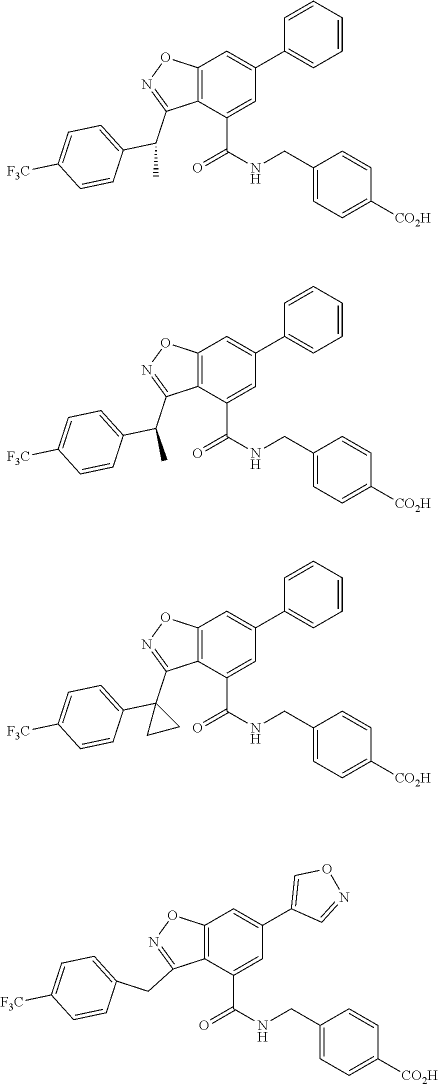 [SMILES: C[C@H](c1ccc(C(F)(F)F)cc1)c1noc2cc(-c3ccccc3)cc(C(=O)NCc3ccc(C(=O)O)cc3)c12 ; C[C@@H](c1ccc(C(F)(F)F)cc1)c1noc2cc(-c3ccccc3)cc(C(=O)NCc3ccc(C(=O)O)cc3)c12 ; O=C(O)c1ccc(CNC(=O)c2cc(-c3ccccc3)cc3onc(C4(c5ccc(C(F)(F)F)cc5)CC4)c23)cc1 ; O=C(O)c1ccc(CNC(=O)c2cc(-c3cnoc3)cc3onc(Cc4ccc(C(F)(F)F)cc4)c23)cc1]

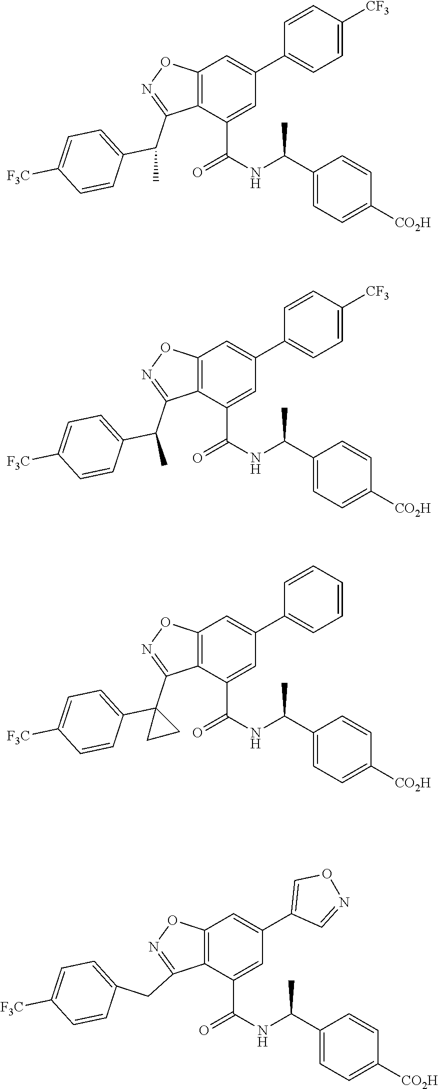 [SMILES: C[C@H](c1ccc(C(F)(F)F)cc1)c1noc2cc(-c3ccc(C(F)(F)F)cc3)cc(C(=O)N[C@@H](C)c3ccc(C(=O)O)cc3)c12 ; C[C@@H](c1ccc(C(F)(F)F)cc1)c1noc2cc(-c3ccc(C(F)(F)F)cc3)cc(C(=O)N[C@@H](C)c3ccc(C(=O)O)cc3)c12 ; C[C@H](NC(=O)c1cc(-c2ccccc2)cc2onc(C3(c4ccc(C(F)(F)F)cc4)CC3)c12)c1ccc(C(=O)O)cc1 ; C[C@H](NC(=O)c1cc(-c2cnoc2)cc2onc(Cc3ccc(C(F)(F)F)cc3)c12)c1ccc(C(=O)O)cc1]

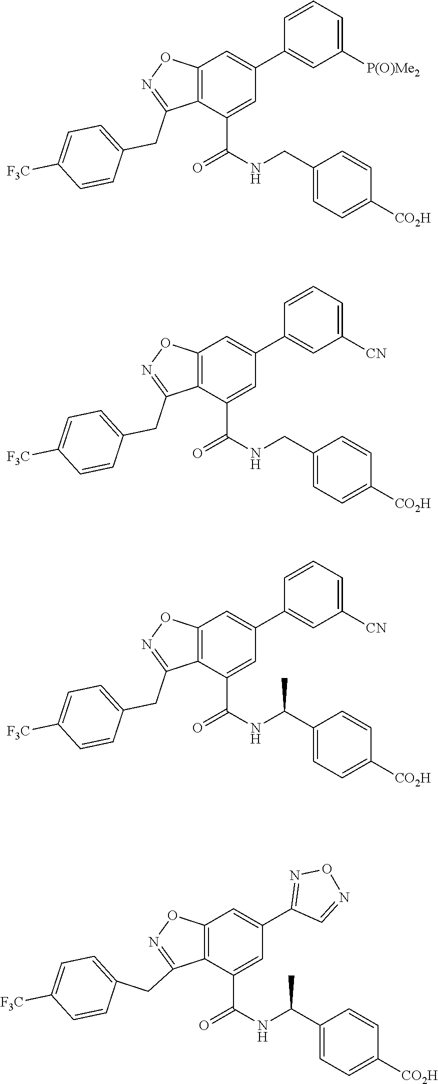

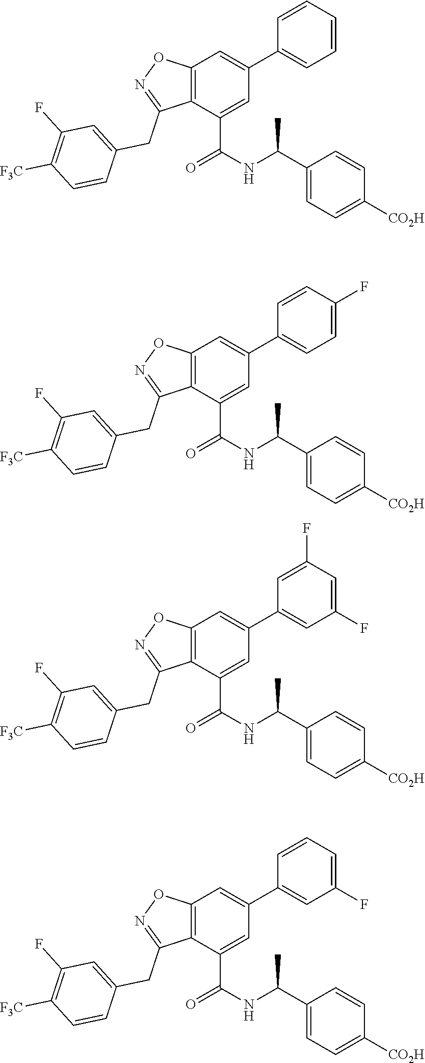

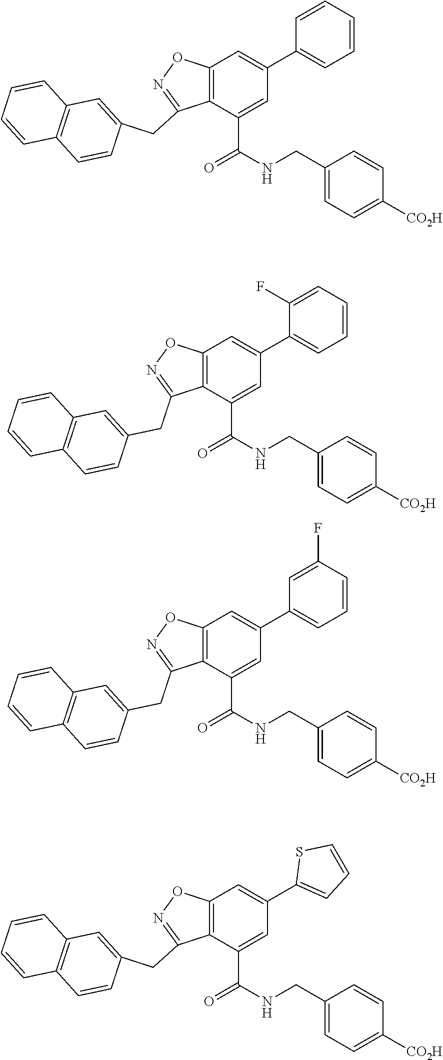

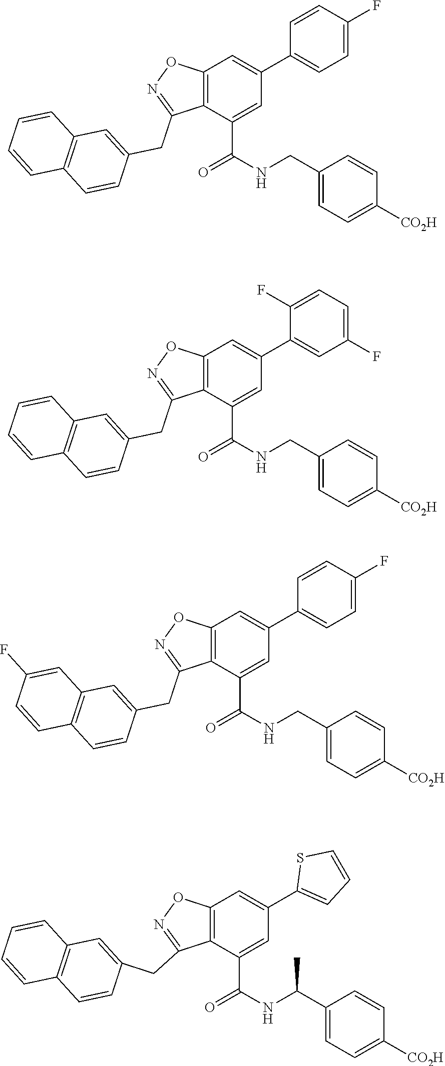 [SMILES: O=C(O)c1ccc(CNC(=O)c2cc(-c3ccc(F)cc3)cc3onc(Cc4ccc5ccccc5c4)c23)cc1 ; O=C(O)c1ccc(CNC(=O)c2cc(-c3cc(F)ccc3F)cc3onc(Cc4ccc5ccccc5c4)c23)cc1 ; O=C(O)c1ccc(CNC(=O)c2cc(-c3ccc(F)cc3)cc3onc(Cc4ccc5ccc(F)cc5c4)c23)cc1 ; C[C@H](NC(=O)c1cc(-c2cccs2)cc2onc(Cc3ccc4ccccc4c3)c12)c1ccc(C(=O)O)cc1]

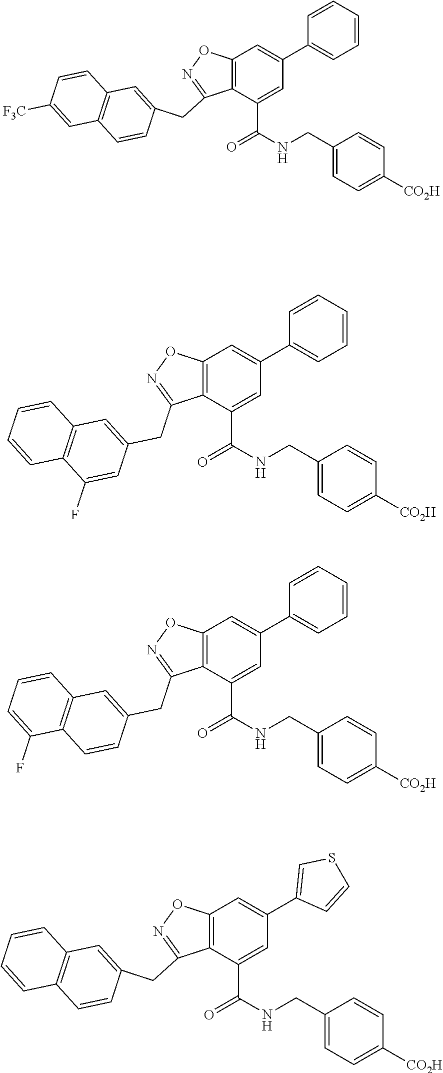 [SMILES: O=C(O)c1ccc(CNC(=O)c2cc(-c3ccccc3)cc3onc(Cc4ccc5cc(C(F)(F)F)ccc5c4)c23)cc1 ; O=C(O)c1ccc(CNC(=O)c2cc(-c3ccccc3)cc3onc(Cc4cc(F)c5ccccc5c4)c23)cc1 ; O=C(O)c1ccc(CNC(=O)c2cc(-c3ccccc3)cc3onc(Cc4ccc5c(F)cccc5c4)c23)cc1 ; O=C(O)c1ccc(CNC(=O)c2cc(-c3ccsc3)cc3onc(Cc4ccc5ccccc5c4)c23)cc1]

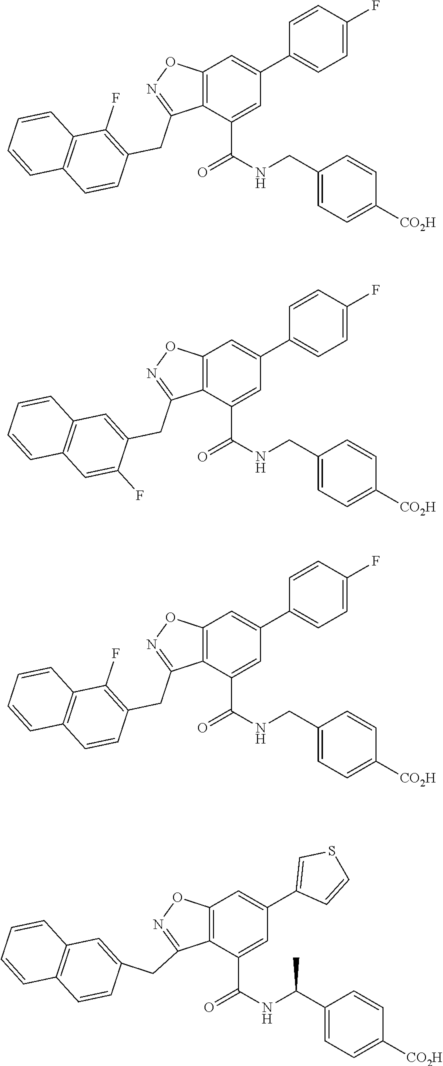 [SMILES: O=C(O)c1ccc(CNC(=O)c2cc(-c3ccc(F)cc3)cc3onc(Cc4ccc5ccccc5c4F)c23)cc1 ; O=C(O)c1ccc(CNC(=O)c2cc(-c3ccc(F)cc3)cc3onc(Cc4cc5ccccc5cc4F)c23)cc1 ; O=C(O)c1ccc(CNC(=O)c2cc(-c3ccc(F)cc3)cc3onc(Cc4ccc5ccccc5c4F)c23)cc1 ; C[C@H](NC(=O)c1cc(-c2ccsc2)cc2onc(Cc3ccc4ccccc4c3)c12)c1ccc(C(=O)O)cc1]

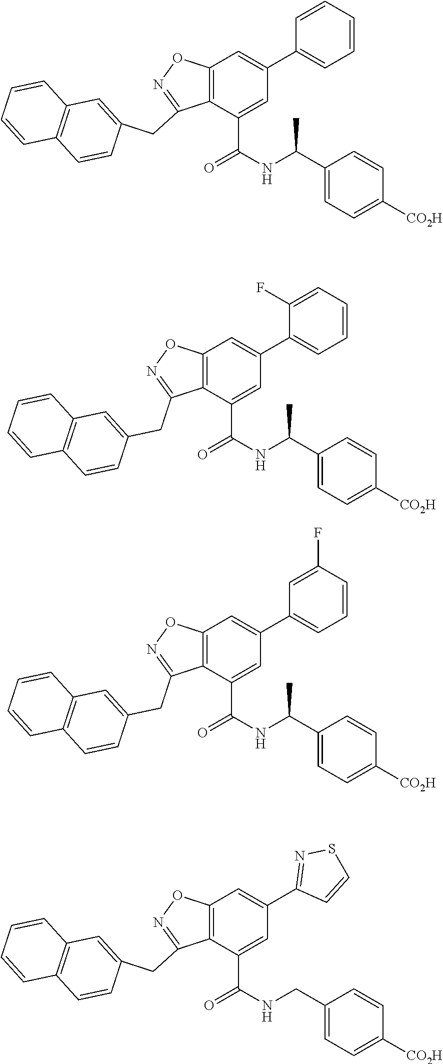

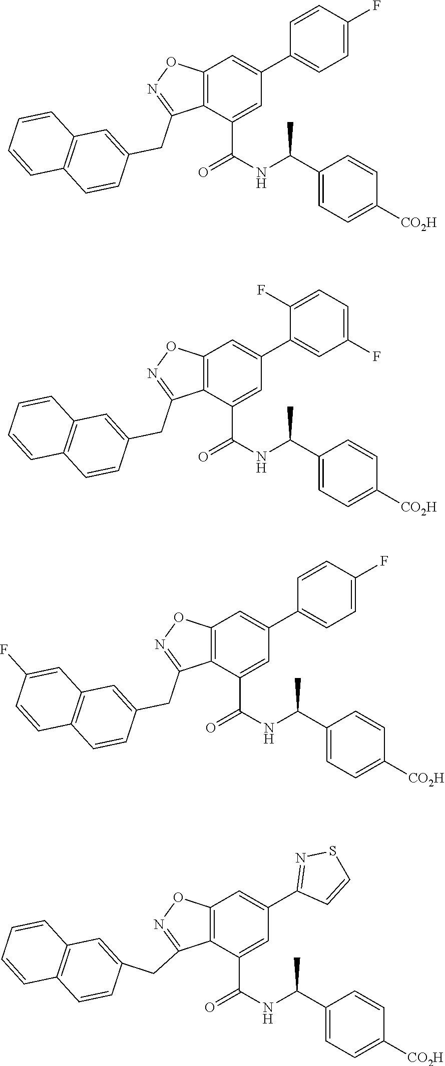 [SMILES: C[C@H](NC(=O)c1cc(-c2ccc(F)cc2)cc2onc(Cc3ccc4ccccc4c3)c12)c1ccc(C(=O)O)cc1 ; C[C@H](NC(=O)c1cc(-c2cc(F)ccc2F)cc2onc(Cc3ccc4ccccc4c3)c12)c1ccc(C(=O)O)cc1 ; C[C@H](NC(=O)c1cc(-c2ccc(F)cc2)cc2onc(Cc3ccc4ccc(F)cc4c3)c12)c1ccc(C(=O)O)cc1 ; C[C@H](NC(=O)c1cc(-c2ccsn2)cc2onc(Cc3ccc4ccccc4c3)c12)c1ccc(C(=O)O)cc1]

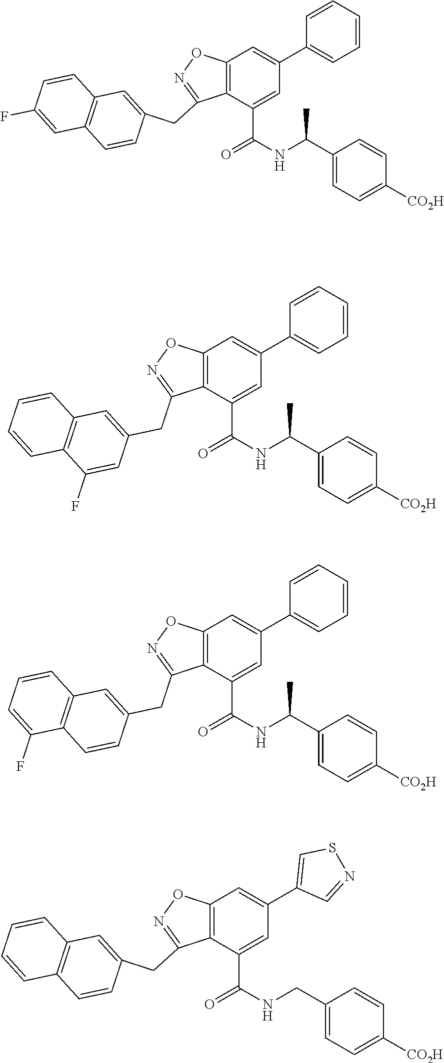 [SMILES: C[C@H](NC(=O)c1cc(-c2ccccc2)cc2onc(Cc3ccc4cc(F)ccc4c3)c12)c1ccc(C(=O)O)cc1 ; C[C@H](NC(=O)c1cc(-c2ccccc2)cc2onc(Cc3cc(F)c4ccccc4c3)c12)c1ccc(C(=O)O)cc1 ; C[C@H](NC(=O)c1cc(-c2ccccc2)cc2onc(Cc3ccc4c(F)cccc4c3)c12)c1ccc(C(=O)O)cc1 ; O=C(O)c1ccc(CNC(=O)c2cc(-c3cnsc3)cc3onc(Cc4ccc5ccccc5c4)c23)cc1]

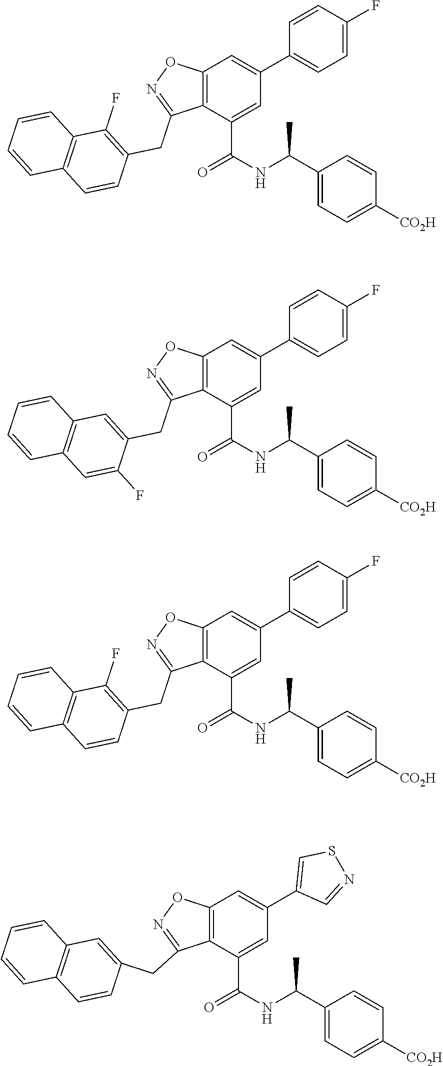 [SMILES: C[C@H](NC(=O)c1cc(-c2ccc(F)cc2)cc2onc(Cc3ccc4ccccc4c3F)c12)c1ccc(C(=O)O)cc1 ; C[C@H](NC(=O)c1cc(-c2ccc(F)cc2)cc2onc(Cc3cc4ccccc4cc3F)c12)c1ccc(C(=O)O)cc1 ; C[C@H](NC(=O)c1cc(-c2ccc(F)cc2)cc2onc(Cc3ccc4ccccc4c3F)c12)c1ccc(C(=O)O)cc1 ; C[C@H](NC(=O)c1cc(-c2cnsc2)cc2onc(Cc3ccc4ccccc4c3)c12)c1ccc(C(=O)O)cc1]

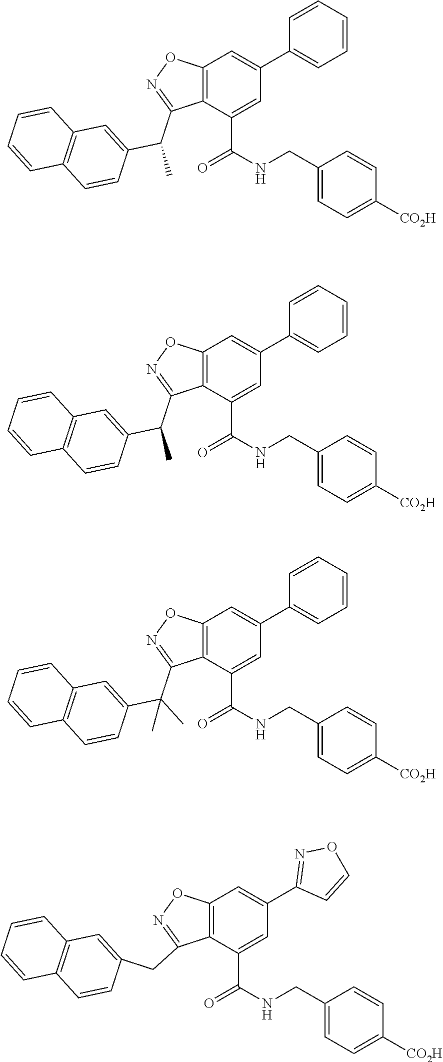 [SMILES: C[C@H](c1ccc2ccccc2c1)c1noc2cc(-c3ccccc3)cc(C(=O)NCc3ccc(C(=O)O)cc3)c12 ; C[C@@H](c1ccc2ccccc2c1)c1noc2cc(-c3ccccc3)cc(C(=O)NCc3ccc(C(=O)O)cc3)c12 ; CC(C)(c1ccc2ccccc2c1)c1noc2cc(-c3ccccc3)cc(C(=O)NCc3ccc(C(=O)O)cc3)c12 ; O=C(O)c1ccc(CNC(=O)c2cc(-c3ccon3)cc3onc(Cc4ccc5ccccc5c4)c23)cc1]

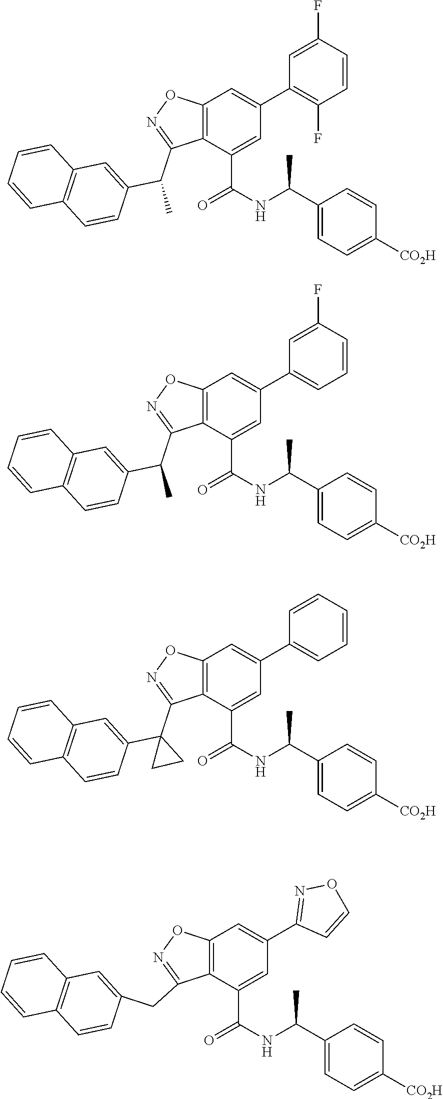

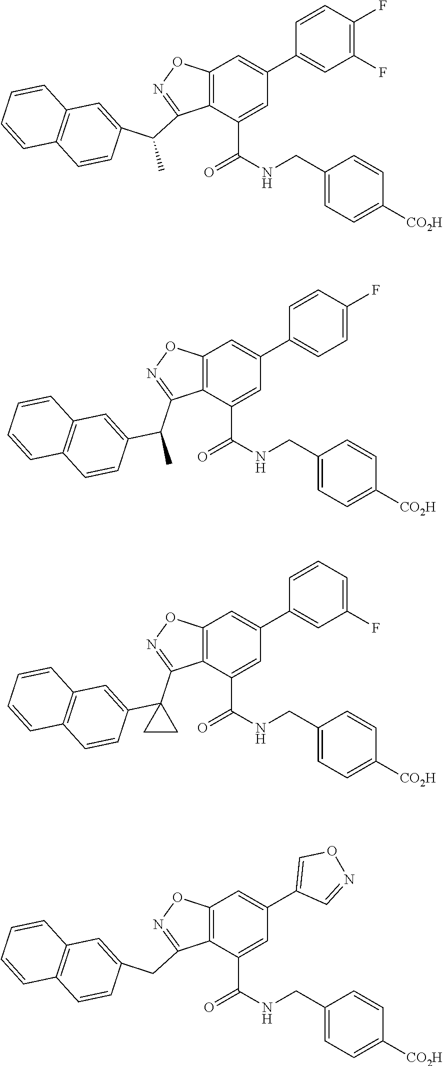 [SMILES: C[C@H](c1ccc2ccccc2c1)c1noc2cc(-c3ccc(F)c(F)c3)cc(C(=O)NCc3ccc(C(=O)O)cc3)c12 ; C[C@@H](c1ccc2ccccc2c1)c1noc2cc(-c3ccc(F)cc3)cc(C(=O)NCc3ccc(C(=O)O)cc3)c12 ; O=C(O)c1ccc(CNC(=O)c2cc(-c3cccc(F)c3)cc3onc(C4(c5ccc6ccccc6c5)CC4)c23)cc1 ; O=C(O)c1ccc(CNC(=O)c2cc(-c3cnoc3)cc3onc(Cc4ccc5ccccc5c4)c23)cc1]

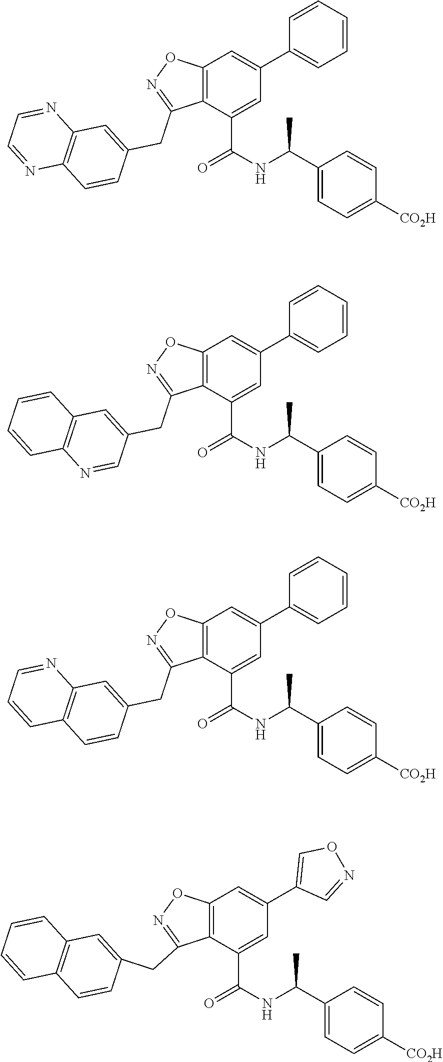 [SMILES: C[C@H](NC(=O)c1cc(-c2ccccc2)cc2onc(Cc3ccc4nccnc4c3)c12)c1ccc(C(=O)O)cc1 ; C[C@H](NC(=O)c1cc(-c2ccccc2)cc2onc(Cc3cnc4ccccc4c3)c12)c1ccc(C(=O)O)cc1 ; C[C@H](NC(=O)c1cc(-c2ccccc2)cc2onc(Cc3ccc4cccnc4c3)c12)c1ccc(C(=O)O)cc1 ; C[C@H](NC(=O)c1cc(-c2cnoc2)cc2onc(Cc3ccc4ccccc4c3)c12)c1ccc(C(=O)O)cc1]

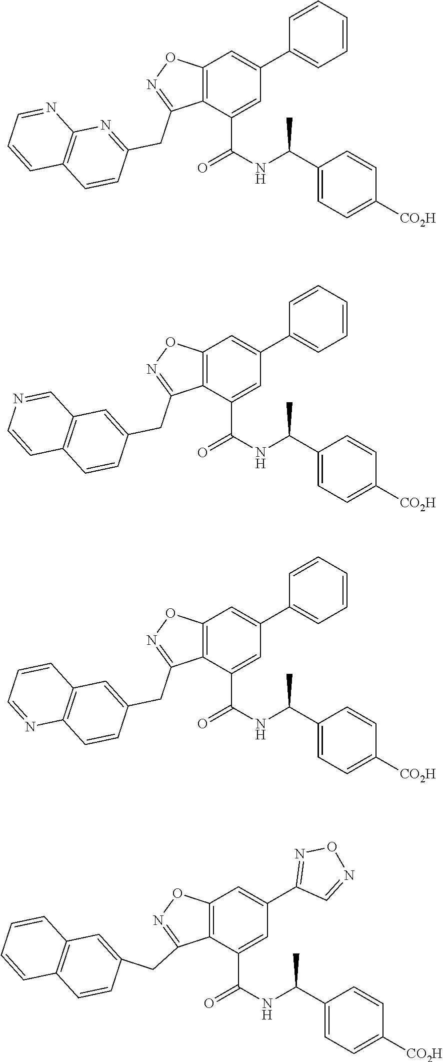 [SMILES: C[C@H](NC(=O)c1cc(-c2ccccc2)cc2onc(Cc3ccc4cccnc4n3)c12)c1ccc(C(=O)O)cc1 ; C[C@H](NC(=O)c1cc(-c2ccccc2)cc2onc(Cc3ccc4ccncc4c3)c12)c1ccc(C(=O)O)cc1 ; C[C@H](NC(=O)c1cc(-c2ccccc2)cc2onc(Cc3ccc4ncccc4c3)c12)c1ccc(C(=O)O)cc1 ; C[C@H](NC(=O)c1cc(-c2cnon2)cc2onc(Cc3ccc4ccccc4c3)c12)c1ccc(C(=O)O)cc1]

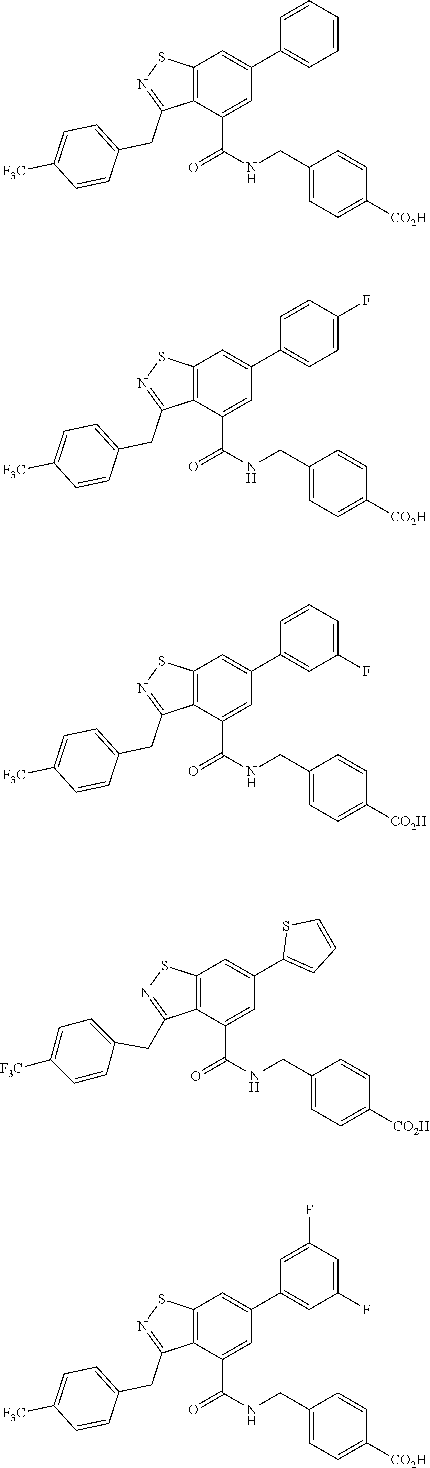 [SMILES: O=C(O)c1ccc(CNC(=O)c2cc(-c3ccccc3)cc3snc(Cc4ccc(C(F)(F)F)cc4)c23)cc1 ; O=C(O)c1ccc(CNC(=O)c2cc(-c3ccc(F)cc3)cc3snc(Cc4ccc(C(F)(F)F)cc4)c23)cc1 ; O=C(O)c1ccc(CNC(=O)c2cc(-c3cccc(F)c3)cc3snc(Cc4ccc(C(F)(F)F)cc4)c23)cc1 ; O=C(O)c1ccc(CNC(=O)c2cc(-c3cccs3)cc3snc(Cc4ccc(C(F)(F)F)cc4)c23)cc1 ; O=C(O)c1ccc(CNC(=O)c2cc(-c3cc(F)cc(F)c3)cc3snc(Cc4ccc(C(F)(F)F)cc4)c23)cc1]

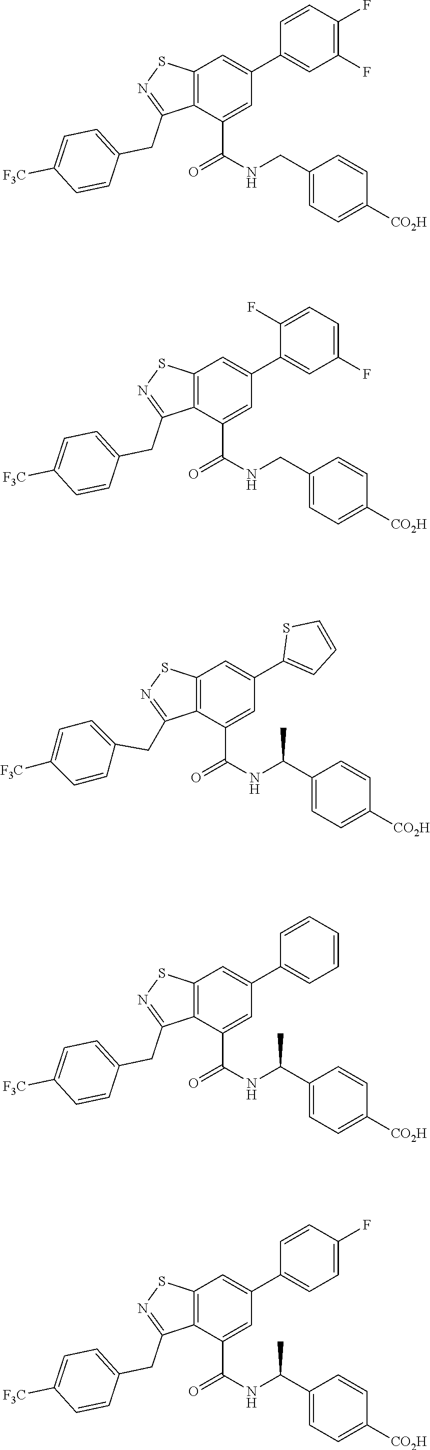 [SMILES: O=C(O)c1ccc(CNC(=O)c2cc(-c3ccc(F)c(F)c3)cc3snc(Cc4ccc(C(F)(F)F)cc4)c23)cc1 ; O=C(O)c1ccc(CNC(=O)c2cc(-c3cc(F)ccc3F)cc3snc(Cc4ccc(C(F)(F)F)cc4)c23)cc1 ; C[C@H](NC(=O)c1cc(-c2cccs2)cc2snc(Cc3ccc(C(F)(F)F)cc3)c12)c1ccc(C(=O)O)cc1 ; C[C@H](NC(=O)c1cc(-c2ccccc2)cc2snc(Cc3ccc(C(F)(F)F)cc3)c12)c1ccc(C(=O)O)cc1 ; C[C@H](NC(=O)c1cc(-c2ccc(F)cc2)cc2snc(Cc3ccc(C(F)(F)F)cc3)c12)c1ccc(C(=O)O)cc1]

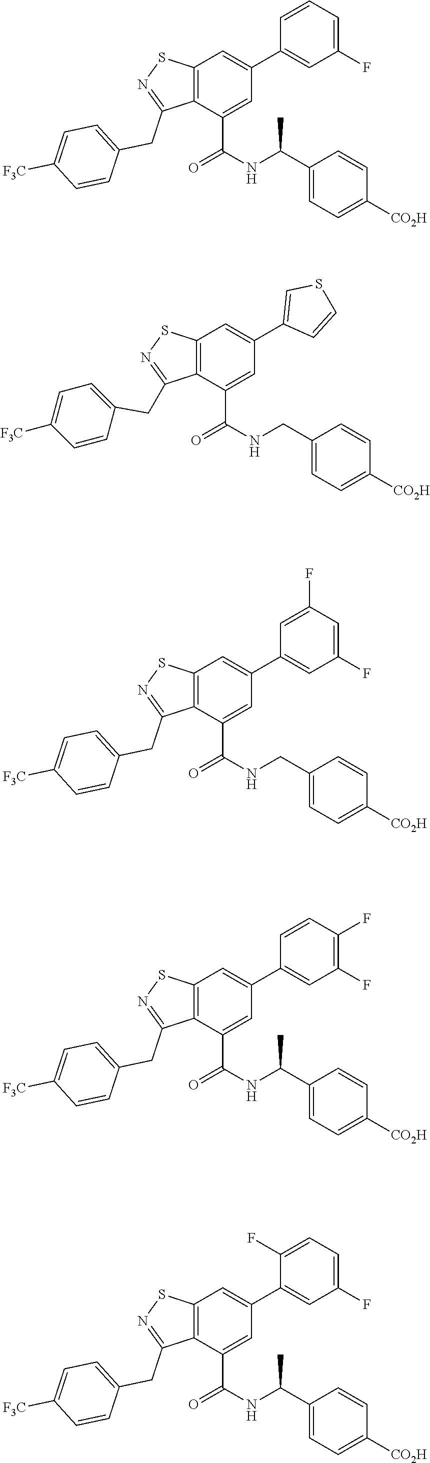 [SMILES: C[C@H](NC(=O)c1cc(-c2cccc(F)c2)cc2snc(Cc3ccc(C(F)(F)F)cc3)c12)c1ccc(C(=O)O)cc1 ; O=C(O)c1ccc(CNC(=O)c2cc(-c3ccsc3)cc3snc(Cc4ccc(C(F)(F)F)cc4)c23)cc1 ; O=C(O)c1ccc(CNC(=O)c2cc(-c3cc(F)cc(F)c3)cc3snc(Cc4ccc(C(F)(F)F)cc4)c23)cc1 ; C[C@H](NC(=O)c1cc(-c2ccc(F)c(F)c2)cc2snc(Cc3ccc(C(F)(F)F)cc3)c12)c1ccc(C(=O)O)cc1 ; C[C@H](NC(=O)c1cc(-c2cc(F)ccc2F)cc2snc(Cc3ccc(C(F)(F)F)cc3)c12)c1ccc(C(=O)O)cc1]

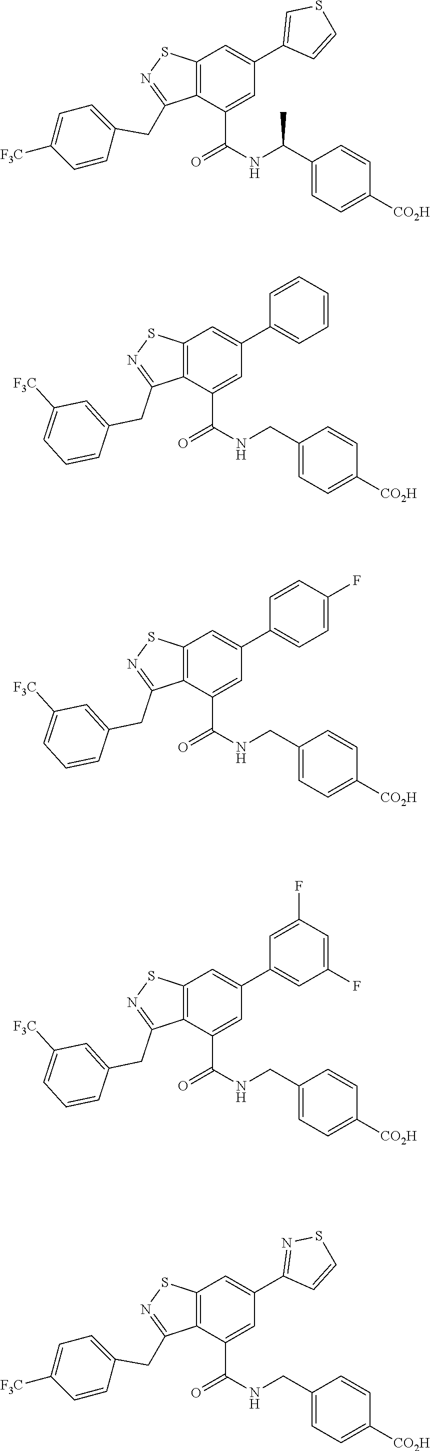 [SMILES: C[C@H](NC(=O)c1cc(-c2ccsc2)cc2snc(Cc3ccc(C(F)(F)F)cc3)c12)c1ccc(C(=O)O)cc1 ; O=C(O)c1ccc(CNC(=O)c2cc(-c3ccccc3)cc3snc(Cc4cccc(C(F)(F)F)c4)c23)cc1 ; O=C(O)c1ccc(CNC(=O)c2cc(-c3ccc(F)cc3)cc3snc(Cc4cccc(C(F)(F)F)c4)c23)cc1 ; O=C(O)c1ccc(CNC(=O)c2cc(-c3cc(F)cc(F)c3)cc3snc(Cc4cccc(C(F)(F)F)c4)c23)cc1 ; O=C(O)c1ccc(CNC(=O)c2cc(-c3ccsn3)cc3snc(Cc4ccc(C(F)(F)F)cc4)c23)cc1]

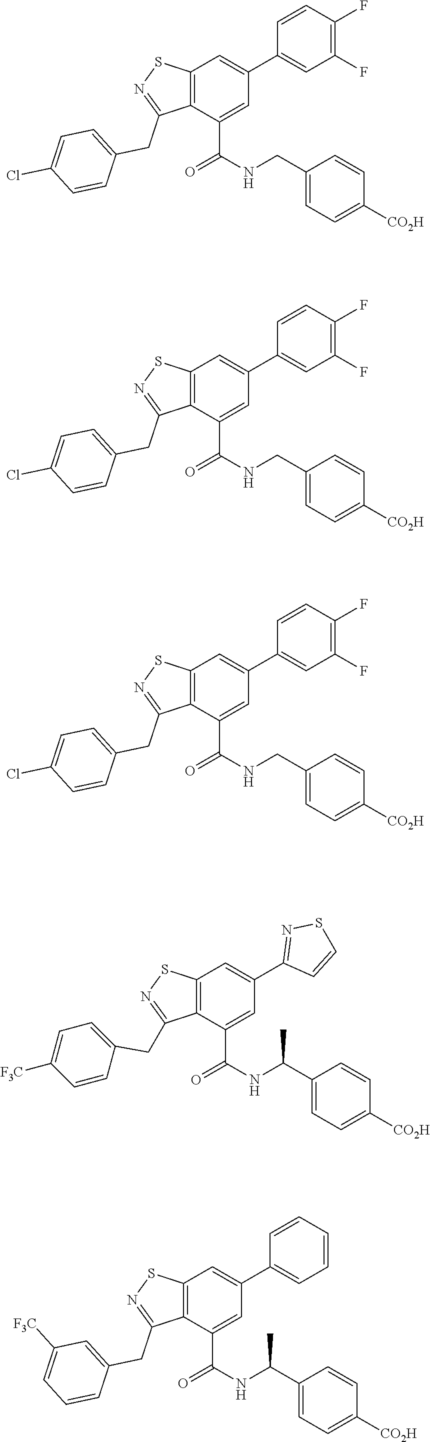 [SMILES: O=C(O)c1ccc(CNC(=O)c2cc(-c3ccc(F)c(F)c3)cc3snc(Cc4ccc(Cl)cc4)c23)cc1 ; O=C(O)c1ccc(CNC(=O)c2cc(-c3ccc(F)c(F)c3)cc3snc(Cc4ccc(Cl)cc4)c23)cc1 ; O=C(O)c1ccc(CNC(=O)c2cc(-c3ccc(F)c(F)c3)cc3snc(Cc4ccc(Cl)cc4)c23)cc1 ; C[C@H](NC(=O)c1cc(-c2ccsn2)cc2snc(Cc3ccc(C(F)(F)F)cc3)c12)c1ccc(C(=O)O)cc1 ; C[C@H](NC(=O)c1cc(-c2ccccc2)cc2snc(Cc3cccc(C(F)(F)F)c3)c12)c1ccc(C(=O)O)cc1]

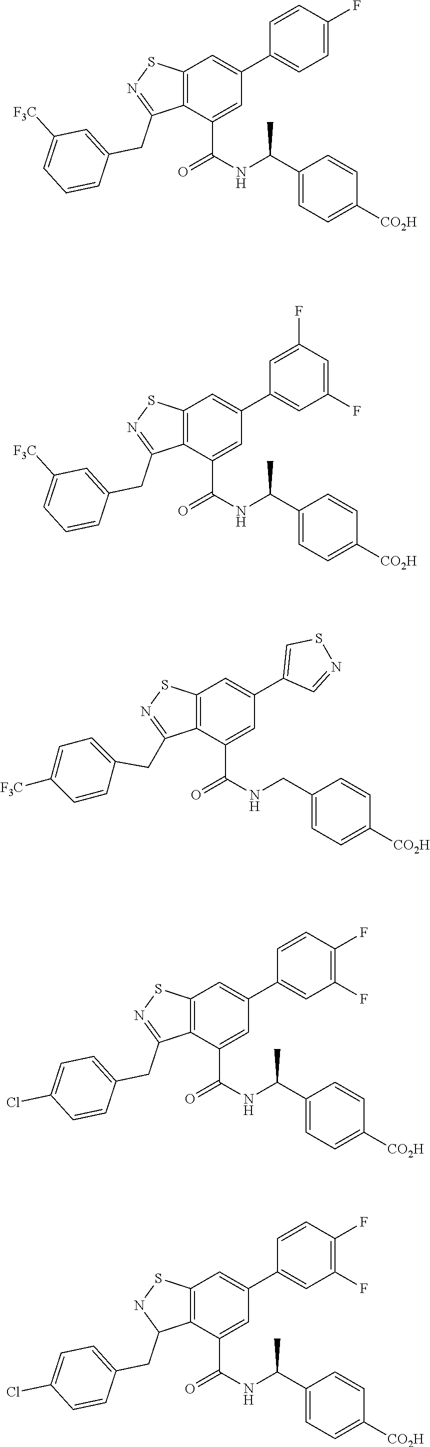 [SMILES: C[C@H](NC(=O)c1cc(-c2ccc(F)cc2)cc2snc(Cc3cccc(C(F)(F)F)c3)c12)c1ccc(C(=O)O)cc1 ; C[C@H](NC(=O)c1cc(-c2cc(F)cc(F)c2)cc2snc(Cc3cccc(C(F)(F)F)c3)c12)c1ccc(C(=O)O)cc1 ; O=C(O)c1ccc(CNC(=O)c2cc(-c3cnsc3)cc3snc(Cc4ccc(C(F)(F)F)cc4)c23)cc1 ; C[C@H](NC(=O)c1cc(-c2ccc(F)c(F)c2)cc2snc(Cc3ccc(Cl)cc3)c12)c1ccc(C(=O)O)cc1 ; C[C@H](NC(=O)c1cc(-c2ccc(F)c(F)c2)cc2c1C(Cc1ccc(Cl)cc1)NS2)c1ccc(C(=O)O)cc1]

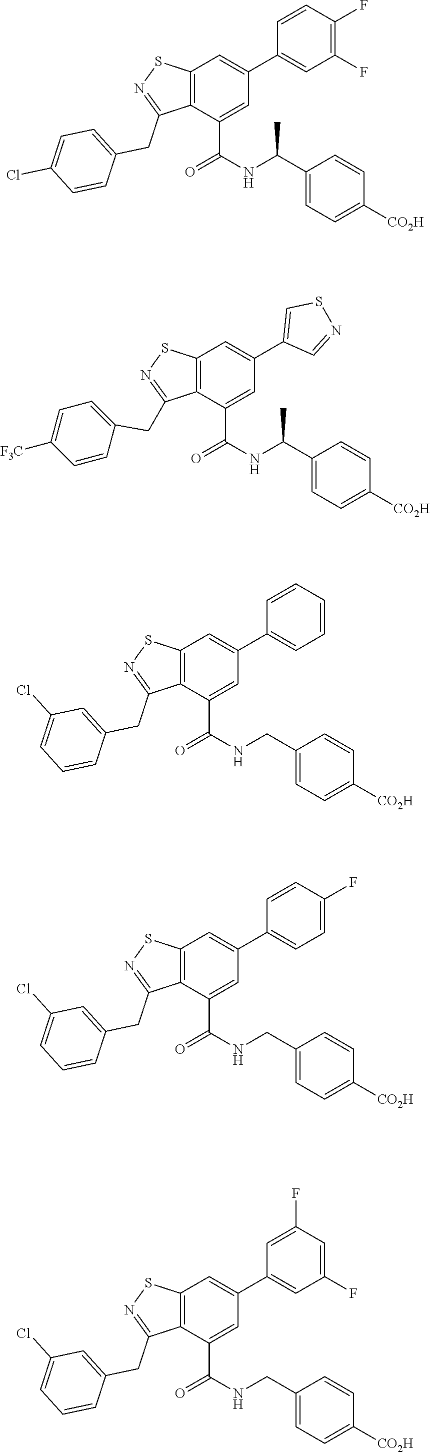 [SMILES: C[C@H](NC(=O)c1cc(-c2ccc(F)c(F)c2)cc2snc(Cc3ccc(Cl)cc3)c12)c1ccc(C(=O)O)cc1 ; C[C@H](NC(=O)c1cc(-c2cnsc2)cc2snc(Cc3ccc(C(F)(F)F)cc3)c12)c1ccc(C(=O)O)cc1 ; O=C(O)c1ccc(CNC(=O)c2cc(-c3ccccc3)cc3snc(Cc4cccc(Cl)c4)c23)cc1 ; O=C(O)c1ccc(CNC(=O)c2cc(-c3ccc(F)cc3)cc3snc(Cc4cccc(Cl)c4)c23)cc1 ; O=C(O)c1ccc(CNC(=O)c2cc(-c3cc(F)cc(F)c3)cc3snc(Cc4cccc(Cl)c4)c23)cc1]

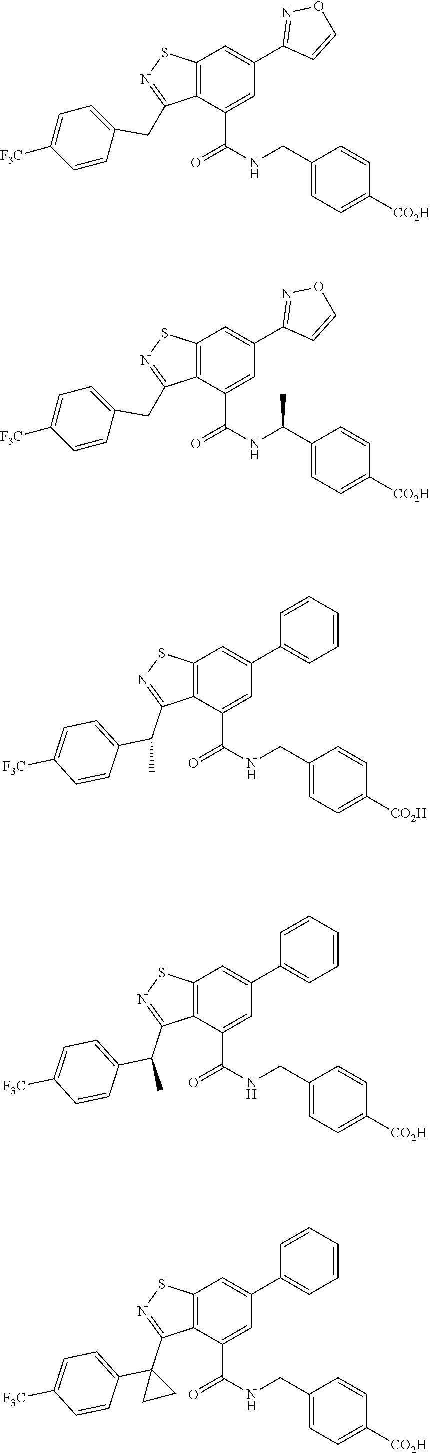 [SMILES: O=C(O)c1ccc(CNC(=O)c2cc(-c3ccon3)cc3snc(Cc4ccc(C(F)(F)F)cc4)c23)cc1 ; C[C@H](NC(=O)c1cc(-c2ccon2)cc2snc(Cc3ccc(C(F)(F)F)cc3)c12)c1ccc(C(=O)O)cc1 ; C[C@H](c1ccc(C(F)(F)F)cc1)c1nsc2cc(-c3ccccc3)cc(C(=O)NCc3ccc(C(=O)O)cc3)c12 ; C[C@@H](c1ccc(C(F)(F)F)cc1)c1nsc2cc(-c3ccccc3)cc(C(=O)NCc3ccc(C(=O)O)cc3)c12 ; O=C(O)c1ccc(CNC(=O)c2cc(-c3ccccc3)cc3snc(C4(c5ccc(C(F)(F)F)cc5)CC4)c23)cc1]

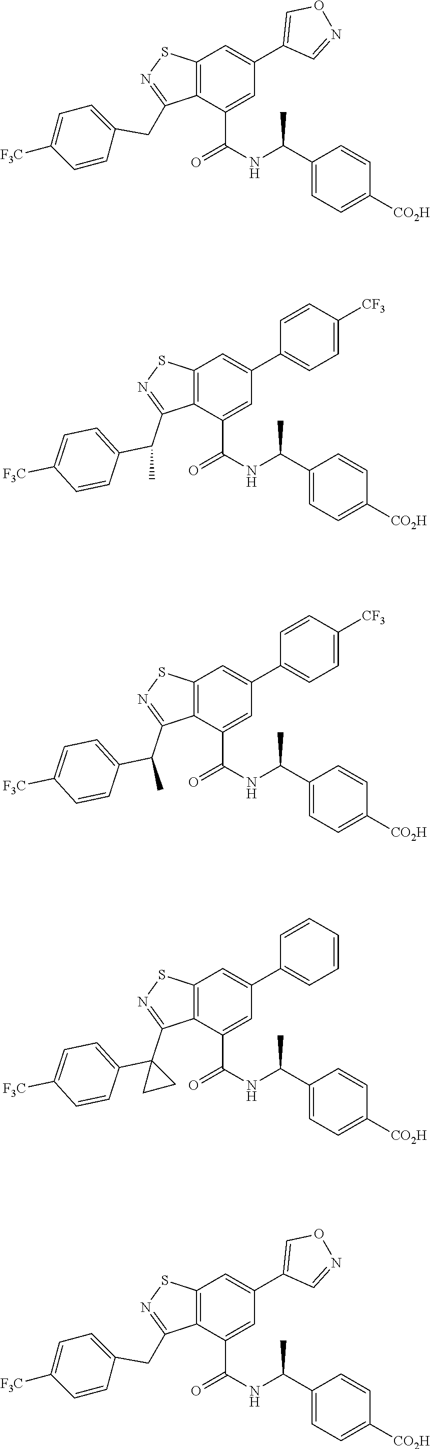 [SMILES: C[C@H](NC(=O)c1cc(-c2cnoc2)cc2snc(Cc3ccc(C(F)(F)F)cc3)c12)c1ccc(C(=O)O)cc1 ; C[C@H](c1ccc(C(F)(F)F)cc1)c1nsc2cc(-c3ccc(C(F)(F)F)cc3)cc(C(=O)N[C@@H](C)c3ccc(C(=O)O)cc3)c12 ; C[C@@H](c1ccc(C(F)(F)F)cc1)c1nsc2cc(-c3ccc(C(F)(F)F)cc3)cc(C(=O)N[C@@H](C)c3ccc(C(=O)O)cc3)c12 ; C[C@H](NC(=O)c1cc(-c2ccccc2)cc2snc(C3(c4ccc(C(F)(F)F)cc4)CC3)c12)c1ccc(C(=O)O)cc1 ; C[C@H](NC(=O)c1cc(-c2cnoc2)cc2snc(Cc3ccc(C(F)(F)F)cc3)c12)c1ccc(C(=O)O)cc1]

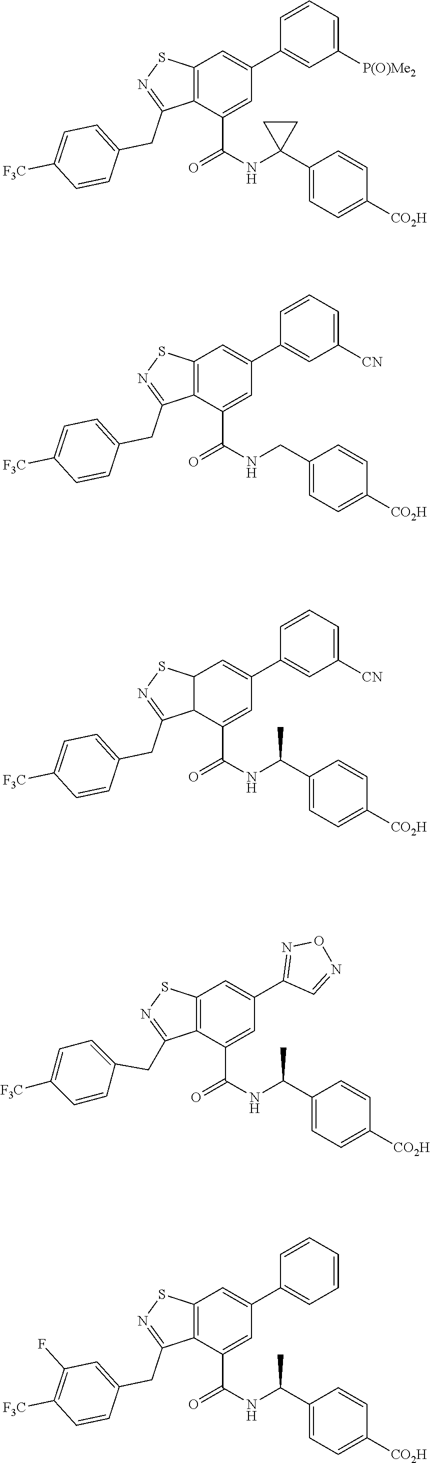

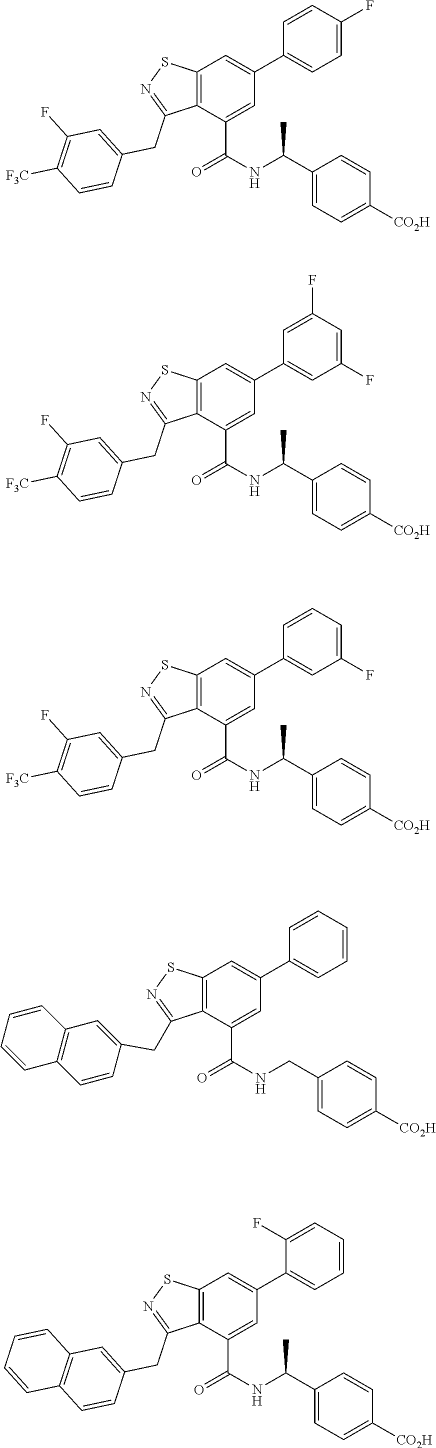 [SMILES: C[C@H](NC(=O)c1cc(-c2ccc(F)cc2)cc2snc(Cc3ccc(C(F)(F)F)c(F)c3)c12)c1ccc(C(=O)O)cc1 ; C[C@H](NC(=O)c1cc(-c2cc(F)cc(F)c2)cc2snc(Cc3ccc(C(F)(F)F)c(F)c3)c12)c1ccc(C(=O)O)cc1 ; C[C@H](NC(=O)c1cc(-c2cccc(F)c2)cc2snc(Cc3ccc(C(F)(F)F)c(F)c3)c12)c1ccc(C(=O)O)cc1 ; O=C(O)c1ccc(CNC(=O)c2cc(-c3ccccc3)cc3snc(Cc4ccc5ccccc5c4)c23)cc1 ; C[C@H](NC(=O)c1cc(-c2ccccc2F)cc2snc(Cc3ccc4ccccc4c3)c12)c1ccc(C(=O)O)cc1]

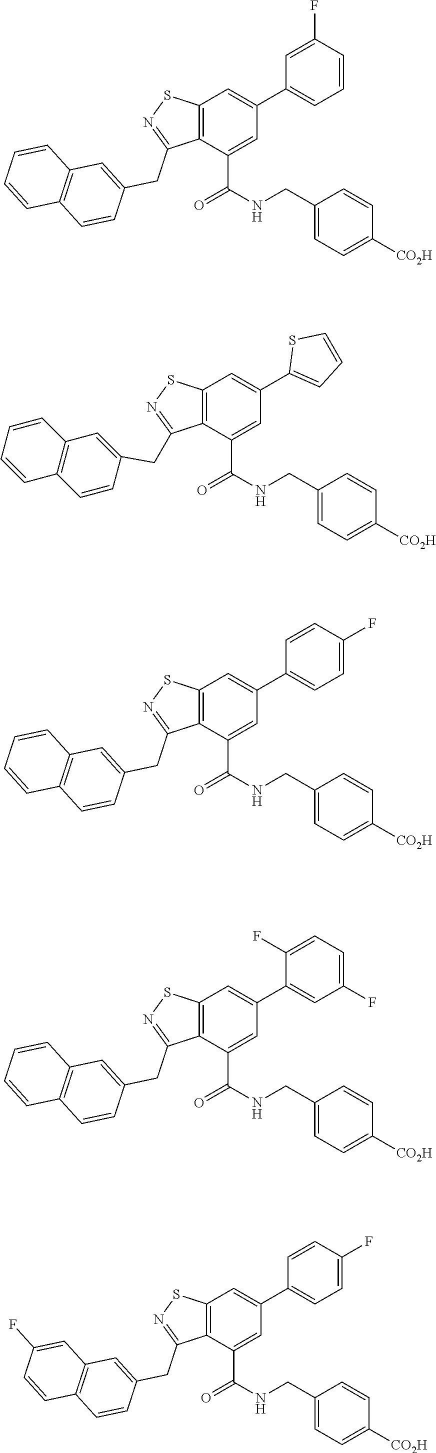 [SMILES: O=C(O)c1ccc(CNC(=O)c2cc(-c3cccc(F)c3)cc3snc(Cc4ccc5ccccc5c4)c23)cc1 ; O=C(O)c1ccc(CNC(=O)c2cc(-c3cccs3)cc3snc(Cc4ccc5ccccc5c4)c23)cc1 ; O=C(O)c1ccc(CNC(=O)c2cc(-c3ccc(F)cc3)cc3snc(Cc4ccc5ccccc5c4)c23)cc1 ; O=C(O)c1ccc(CNC(=O)c2cc(-c3cc(F)ccc3F)cc3snc(Cc4ccc5ccccc5c4)c23)cc1 ; O=C(O)c1ccc(CNC(=O)c2cc(-c3ccc(F)cc3)cc3snc(Cc4ccc5ccc(F)cc5c4)c23)cc1]

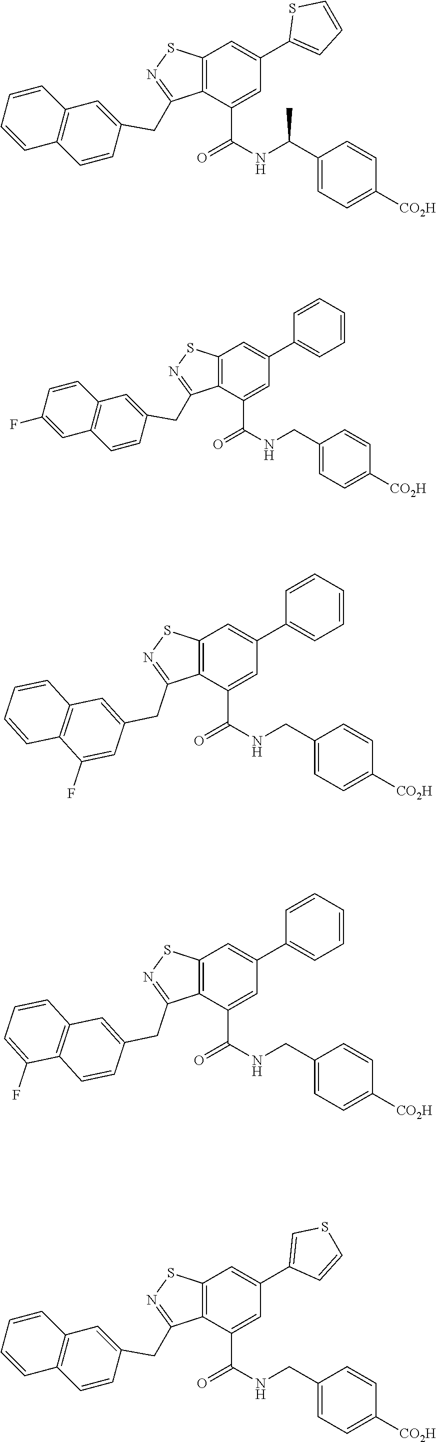 [SMILES: C[C@H](NC(=O)c1cc(-c2cccs2)cc2snc(Cc3ccc4ccccc4c3)c12)c1ccc(C(=O)O)cc1 ; O=C(O)c1ccc(CNC(=O)c2cc(-c3ccccc3)cc3snc(Cc4ccc5cc(F)ccc5c4)c23)cc1 ; O=C(O)c1ccc(CNC(=O)c2cc(-c3ccccc3)cc3snc(Cc4cc(F)c5ccccc5c4)c23)cc1 ; O=C(O)c1ccc(CNC(=O)c2cc(-c3ccccc3)cc3snc(Cc4ccc5c(F)cccc5c4)c23)cc1 ; O=C(O)c1ccc(CNC(=O)c2cc(-c3ccsc3)cc3snc(Cc4ccc5ccccc5c4)c23)cc1]

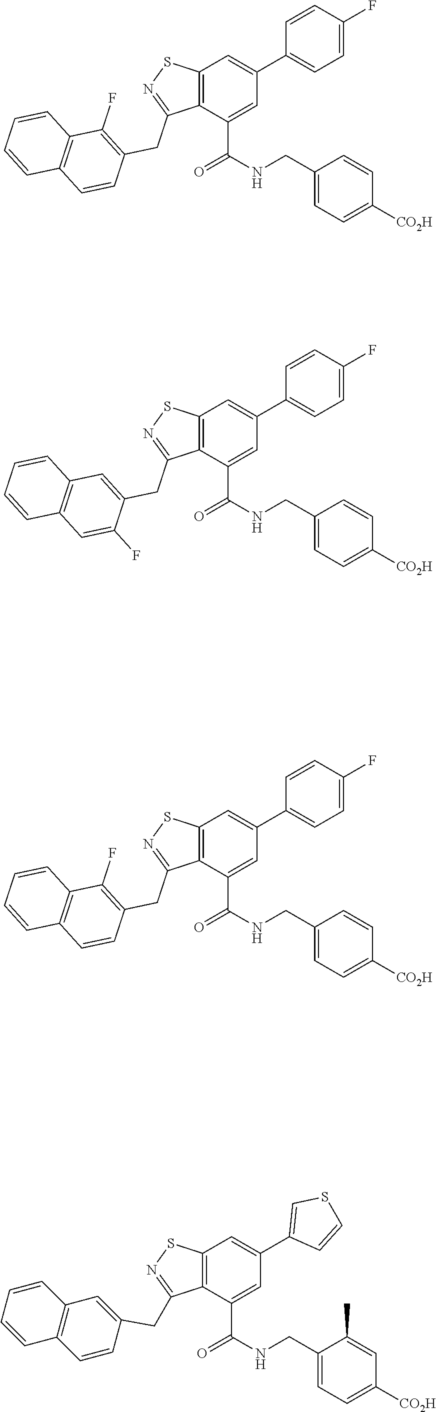

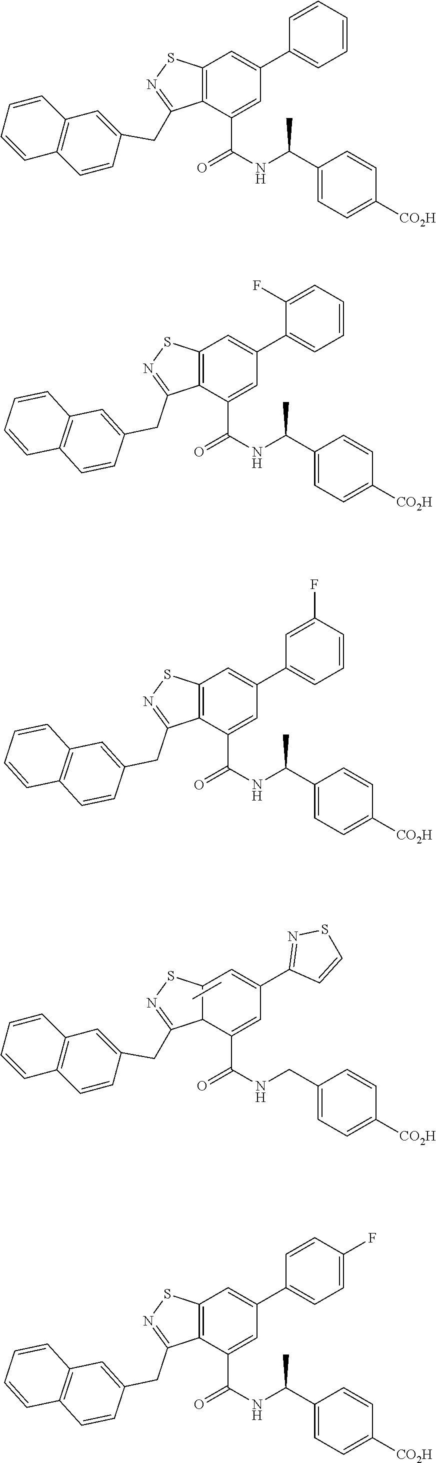

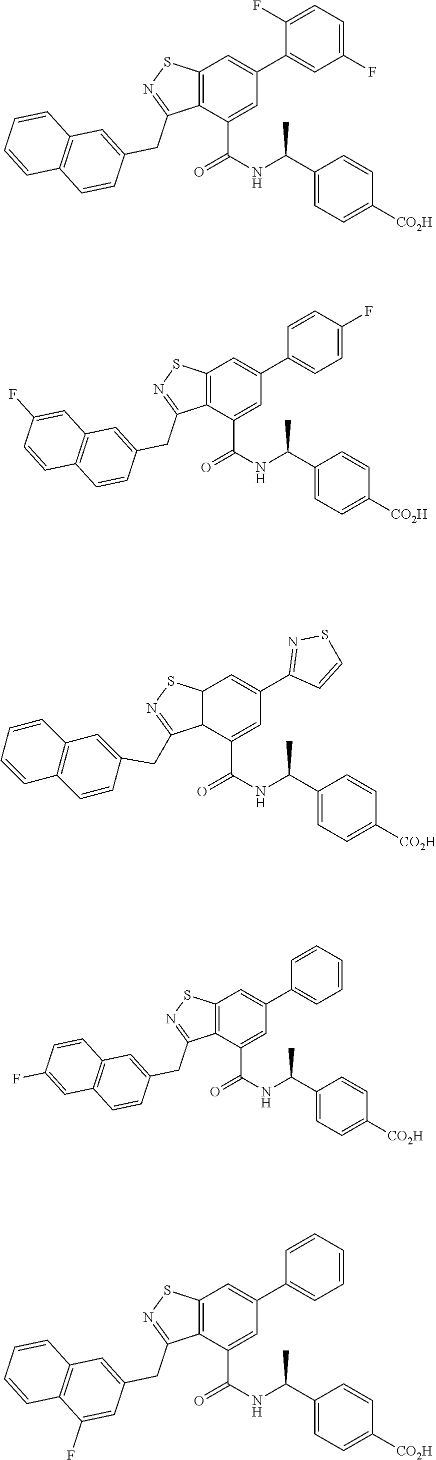 [SMILES: C[C@H](NC(=O)c1cc(-c2cc(F)ccc2F)cc2snc(Cc3ccc4ccccc4c3)c12)c1ccc(C(=O)O)cc1 ; C[C@H](NC(=O)c1cc(-c2ccc(F)cc2)cc2snc(Cc3ccc4ccc(F)cc4c3)c12)c1ccc(C(=O)O)cc1 ; C[C@H](NC(=O)C1=CC(c2ccsn2)=CC2SN=C(Cc3ccc4ccccc4c3)C12)c1ccc(C(=O)O)cc1 ; C[C@H](NC(=O)c1cc(-c2ccccc2)cc2snc(Cc3ccc4cc(F)ccc4c3)c12)c1ccc(C(=O)O)cc1 ; C[C@H](NC(=O)c1cc(-c2ccccc2)cc2snc(Cc3cc(F)c4ccccc4c3)c12)c1ccc(C(=O)O)cc1]

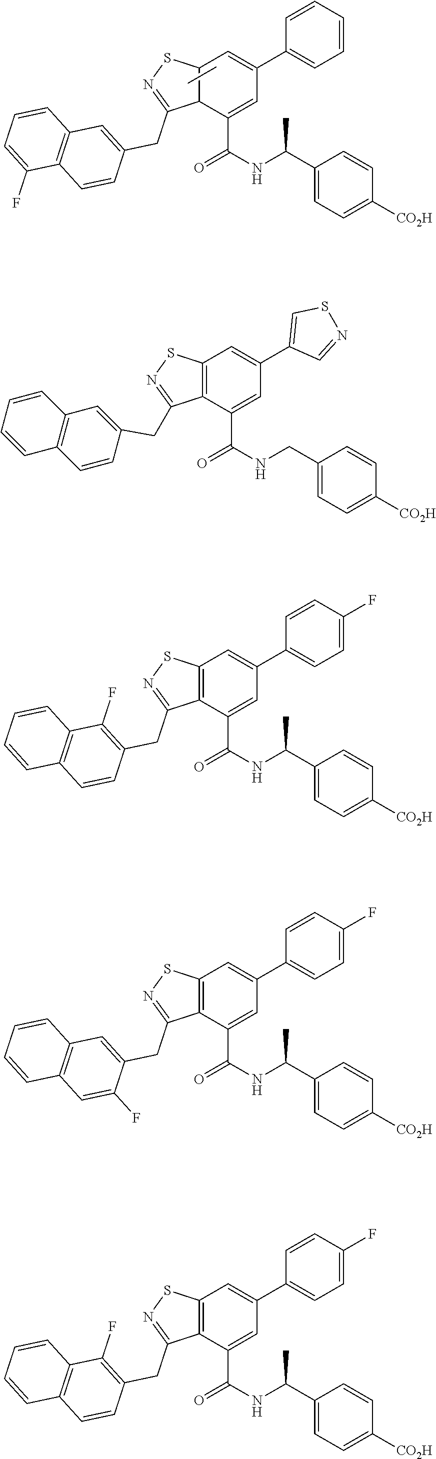

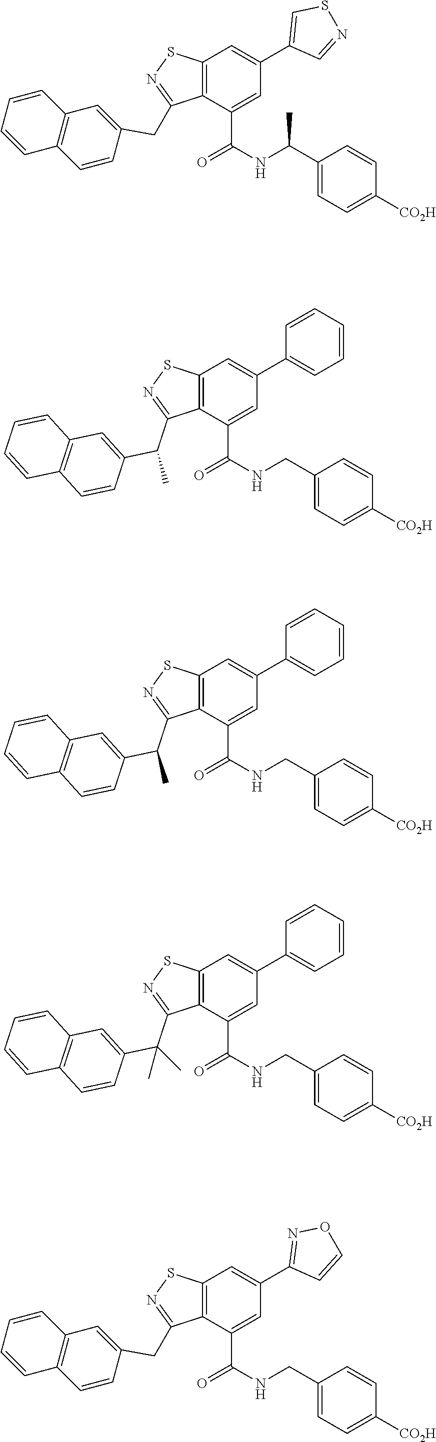 [SMILES: C[C@H](NC(=O)c1cc(-c2cnsc2)cc2snc(Cc3ccc4ccccc4c3)c12)c1ccc(C(=O)O)cc1 ; C[C@H](c1ccc2ccccc2c1)c1nsc2cc(-c3ccccc3)cc(C(=O)NCc3ccc(C(=O)O)cc3)c12 ; C[C@@H](c1ccc2ccccc2c1)c1nsc2cc(-c3ccccc3)cc(C(=O)NCc3ccc(C(=O)O)cc3)c12 ; CC(C)(c1ccc2ccccc2c1)c1nsc2cc(-c3ccccc3)cc(C(=O)NCc3ccc(C(=O)O)cc3)c12 ; O=C(O)c1ccc(CNC(=O)c2cc(-c3ccon3)cc3snc(Cc4ccc5ccccc5c4)c23)cc1]

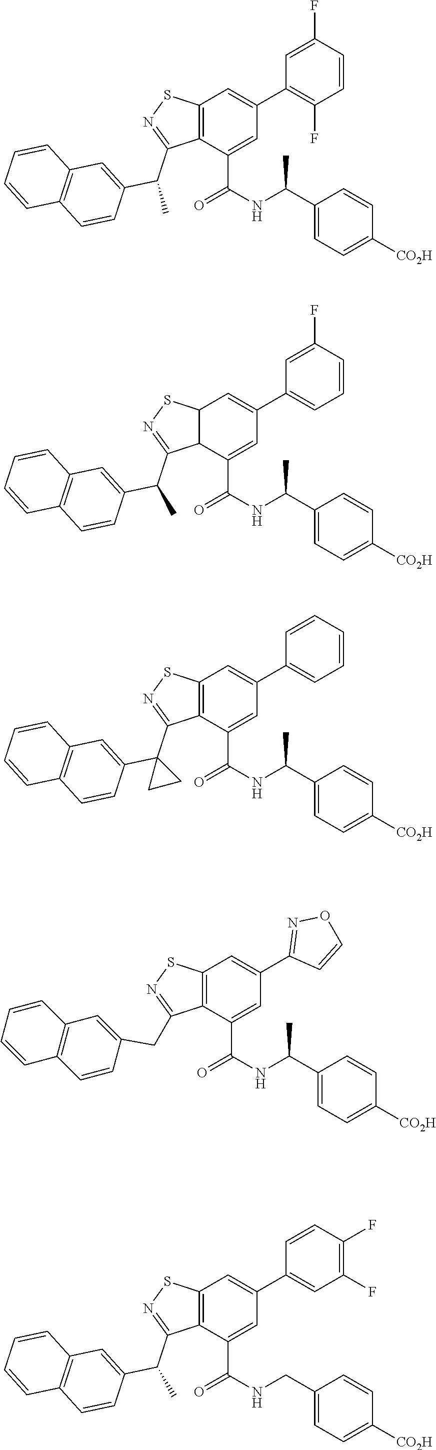 [SMILES: C[C@H](c1ccc2ccccc2c1)c1nsc2cc(-c3cc(F)ccc3F)cc(C(=O)N[C@@H](C)c3ccc(C(=O)O)cc3)c12 ; C[C@H](NC(=O)C1=CC(c2cccc(F)c2)=CC2SN=C([C@@H](C)c3ccc4ccccc4c3)C12)c1ccc(C(=O)O)cc1 ; C[C@H](NC(=O)c1cc(-c2ccccc2)cc2snc(C3(c4ccc5ccccc5c4)CC3)c12)c1ccc(C(=O)O)cc1 ; C[C@H](NC(=O)c1cc(-c2ccon2)cc2snc(Cc3ccc4ccccc4c3)c12)c1ccc(C(=O)O)cc1 ; C[C@H](c1ccc2ccccc2c1)c1nsc2cc(-c3ccc(F)c(F)c3)cc(C(=O)NCc3ccc(C(=O)O)cc3)c12]

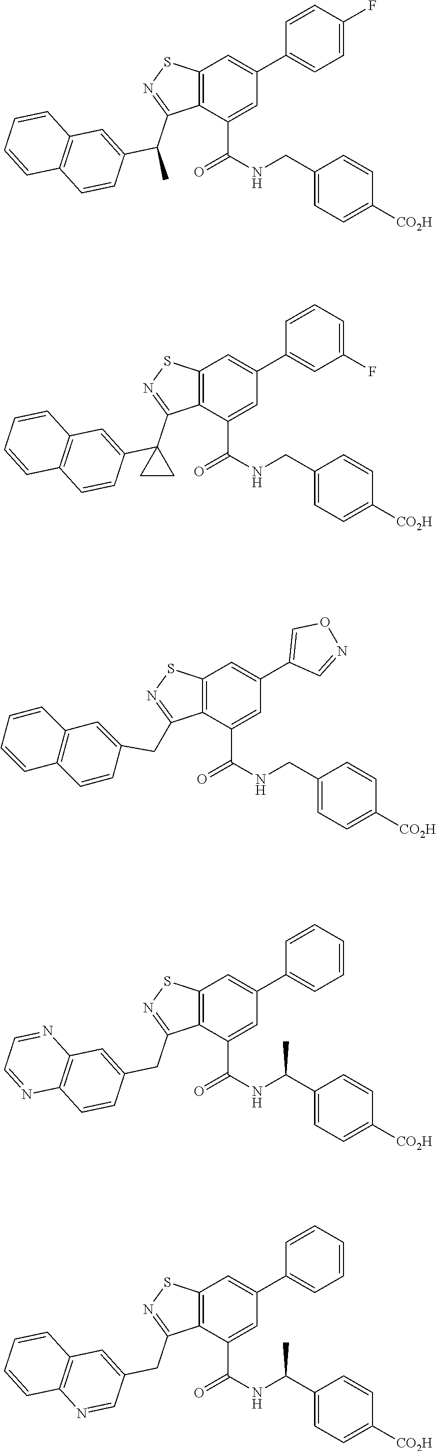 [SMILES: C[C@@H](c1ccc2ccccc2c1)c1nsc2cc(-c3ccc(F)cc3)cc(C(=O)NCc3ccc(C(=O)O)cc3)c12 ; O=C(O)c1ccc(CNC(=O)c2cc(-c3cccc(F)c3)cc3snc(C4(c5ccc6ccccc6c5)CC4)c23)cc1 ; O=C(O)c1ccc(CNC(=O)c2cc(-c3cnoc3)cc3snc(Cc4ccc5ccccc5c4)c23)cc1 ; C[C@H](NC(=O)c1cc(-c2ccccc2)cc2snc(Cc3ccc4nccnc4c3)c12)c1ccc(C(=O)O)cc1 ; C[C@H](NC(=O)c1cc(-c2ccccc2)cc2snc(Cc3cnc4ccccc4c3)c12)c1ccc(C(=O)O)cc1]

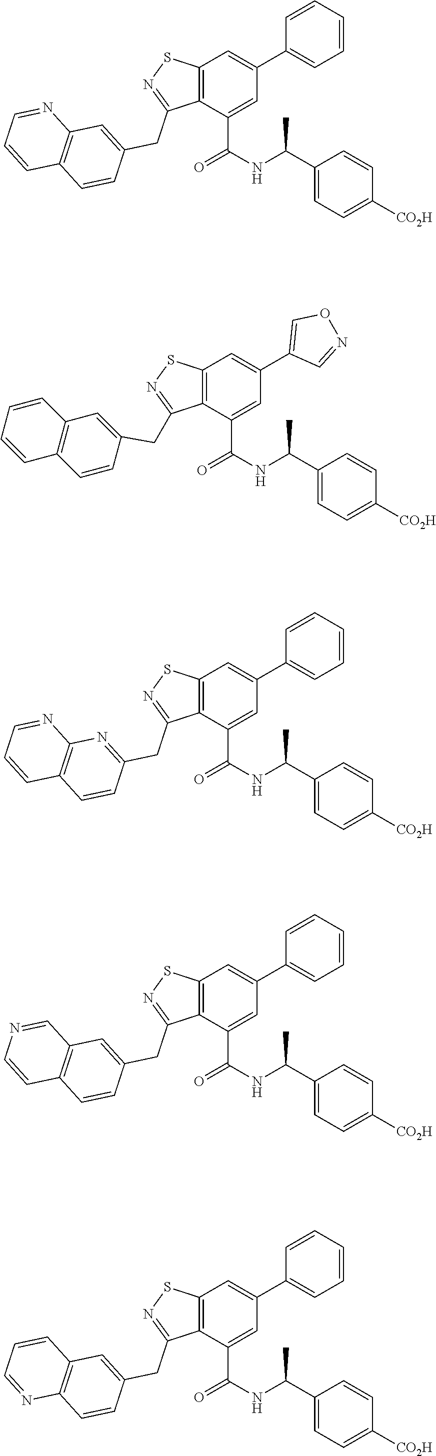 [SMILES: C[C@H](NC(=O)c1cc(-c2ccccc2)cc2snc(Cc3ccc4cccnc4c3)c12)c1ccc(C(=O)O)cc1 ; C[C@H](NC(=O)c1cc(-c2cnoc2)cc2snc(Cc3ccc4ccccc4c3)c12)c1ccc(C(=O)O)cc1 ; C[C@H](NC(=O)c1cc(-c2ccccc2)cc2snc(Cc3ccc4cccnc4n3)c12)c1ccc(C(=O)O)cc1 ; C[C@H](NC(=O)c1cc(-c2ccccc2)cc2snc(Cc3ccc4ccncc4c3)c12)c1ccc(C(=O)O)cc1 ; C[C@H](NC(=O)c1cc(-c2ccccc2)cc2snc(Cc3ccc4ncccc4c3)c12)c1ccc(C(=O)O)cc1]

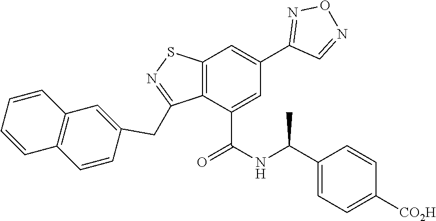 [SMILES: C[C@H](NC(=O)c1cc(-c2cnon2)cc2snc(Cc3ccc4ccccc4c3)c12)c1ccc(C(=O)O)cc1]

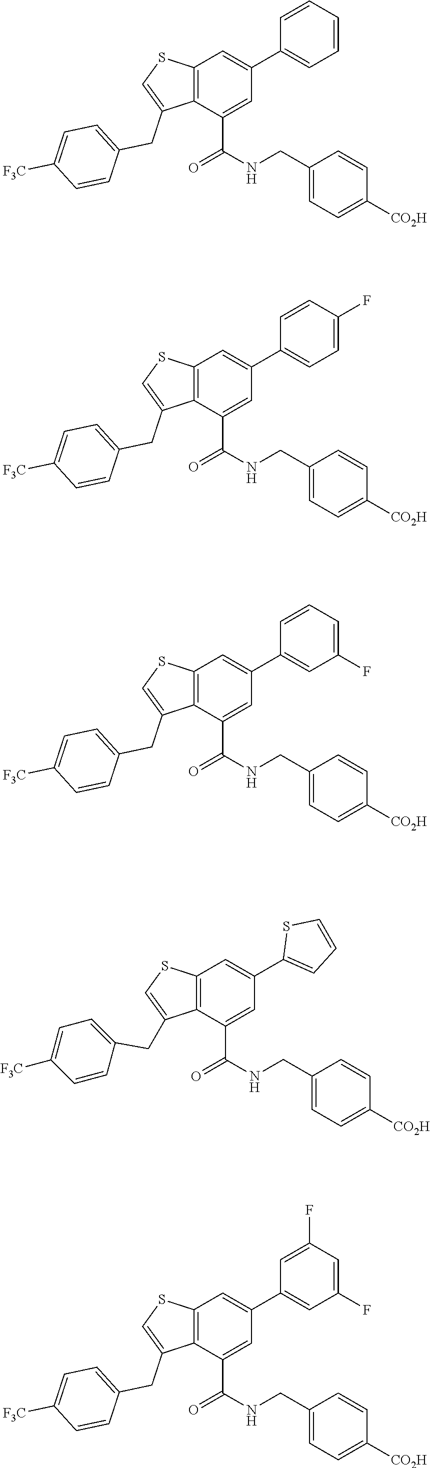 [SMILES: O=C(O)c1ccc(CNC(=O)c2cc(-c3ccccc3)cc3scc(Cc4ccc(C(F)(F)F)cc4)c23)cc1 ; O=C(O)c1ccc(CNC(=O)c2cc(-c3ccc(F)cc3)cc3scc(Cc4ccc(C(F)(F)F)cc4)c23)cc1 ; O=C(O)c1ccc(CNC(=O)c2cc(-c3cccc(F)c3)cc3scc(Cc4ccc(C(F)(F)F)cc4)c23)cc1 ; O=C(O)c1ccc(CNC(=O)c2cc(-c3cccs3)cc3scc(Cc4ccc(C(F)(F)F)cc4)c23)cc1 ; O=C(O)c1ccc(CNC(=O)c2cc(-c3cc(F)cc(F)c3)cc3scc(Cc4ccc(C(F)(F)F)cc4)c23)cc1]

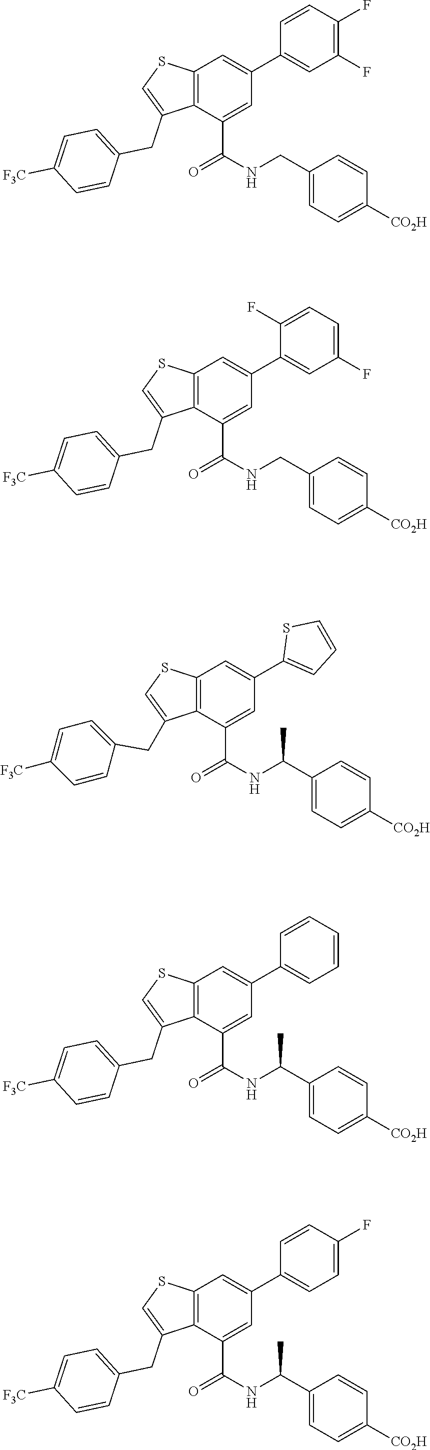 [SMILES: O=C(O)c1ccc(CNC(=O)c2cc(-c3ccc(F)c(F)c3)cc3scc(Cc4ccc(C(F)(F)F)cc4)c23)cc1 ; O=C(O)c1ccc(CNC(=O)c2cc(-c3cc(F)ccc3F)cc3scc(Cc4ccc(C(F)(F)F)cc4)c23)cc1 ; C[C@H](NC(=O)c1cc(-c2cccs2)cc2scc(Cc3ccc(C(F)(F)F)cc3)c12)c1ccc(C(=O)O)cc1 ; C[C@H](NC(=O)c1cc(-c2ccccc2)cc2scc(Cc3ccc(C(F)(F)F)cc3)c12)c1ccc(C(=O)O)cc1 ; C[C@H](NC(=O)c1cc(-c2ccc(F)cc2)cc2scc(Cc3ccc(C(F)(F)F)cc3)c12)c1ccc(C(=O)O)cc1]

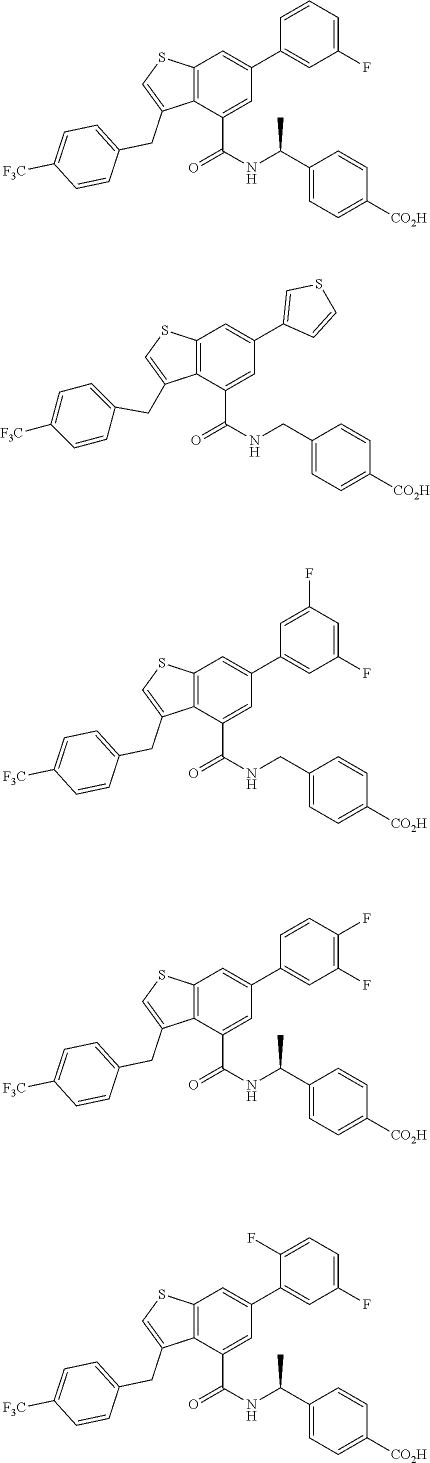 [SMILES: C[C@H](NC(=O)c1cc(-c2cccc(F)c2)cc2scc(Cc3ccc(C(F)(F)F)cc3)c12)c1ccc(C(=O)O)cc1 ; O=C(O)c1ccc(CNC(=O)c2cc(-c3ccsc3)cc3scc(Cc4ccc(C(F)(F)F)cc4)c23)cc1 ; O=C(O)c1ccc(CNC(=O)c2cc(-c3cc(F)cc(F)c3)cc3scc(Cc4ccc(C(F)(F)F)cc4)c23)cc1 ; C[C@H](NC(=O)c1cc(-c2ccc(F)c(F)c2)cc2scc(Cc3ccc(C(F)(F)F)cc3)c12)c1ccc(C(=O)O)cc1 ; C[C@H](NC(=O)c1cc(-c2cc(F)ccc2F)cc2scc(Cc3ccc(C(F)(F)F)cc3)c12)c1ccc(C(=O)O)cc1]

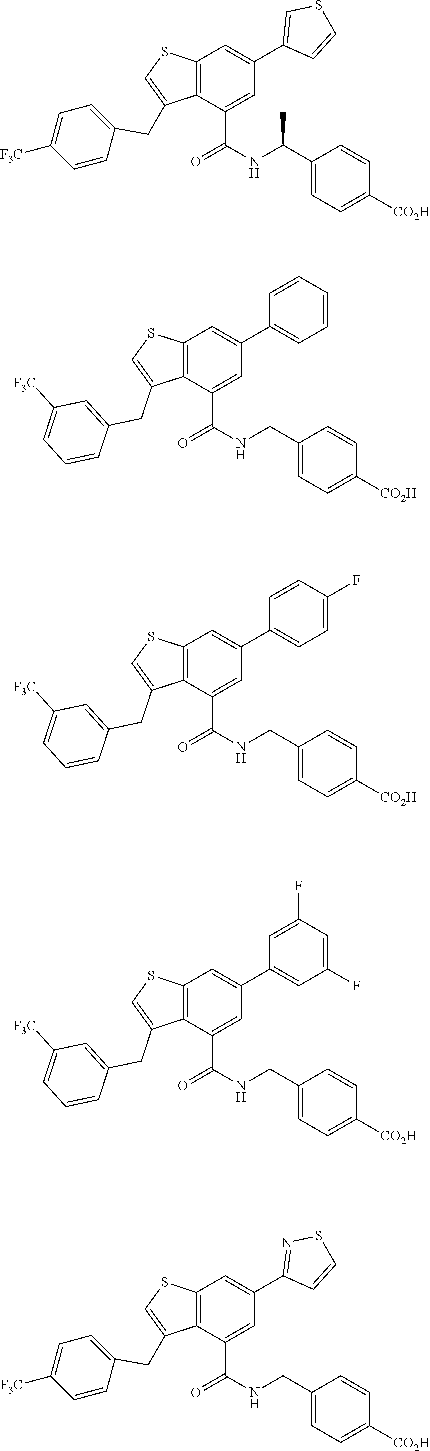 [SMILES: C[C@H](NC(=O)c1cc(-c2ccsc2)cc2scc(Cc3ccc(C(F)(F)F)cc3)c12)c1ccc(C(=O)O)cc1 ; O=C(O)c1ccc(CNC(=O)c2cc(-c3ccccc3)cc3scc(Cc4cccc(C(F)(F)F)c4)c23)cc1 ; O=C(O)c1ccc(CNC(=O)c2cc(-c3ccc(F)cc3)cc3scc(Cc4cccc(C(F)(F)F)c4)c23)cc1 ; O=C(O)c1ccc(CNC(=O)c2cc(-c3cc(F)cc(F)c3)cc3scc(Cc4cccc(C(F)(F)F)c4)c23)cc1 ; O=C(O)c1ccc(CNC(=O)c2cc(-c3ccsn3)cc3scc(Cc4ccc(C(F)(F)F)cc4)c23)cc1]

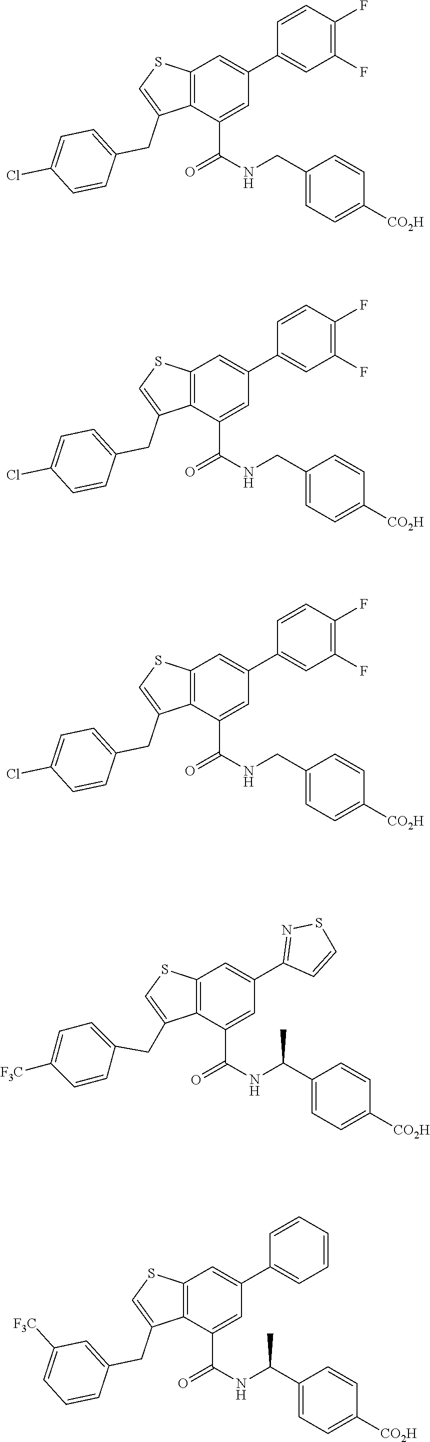 [SMILES: O=C(O)c1ccc(CNC(=O)c2cc(-c3ccc(F)c(F)c3)cc3scc(Cc4ccc(Cl)cc4)c23)cc1 ; O=C(O)c1ccc(CNC(=O)c2cc(-c3ccc(F)c(F)c3)cc3scc(Cc4ccc(Cl)cc4)c23)cc1 ; O=C(O)c1ccc(CNC(=O)c2cc(-c3ccc(F)c(F)c3)cc3scc(Cc4ccc(Cl)cc4)c23)cc1 ; C[C@H](NC(=O)c1cc(-c2ccsn2)cc2scc(Cc3ccc(C(F)(F)F)cc3)c12)c1ccc(C(=O)O)cc1 ; C[C@H](NC(=O)c1cc(-c2ccccc2)cc2scc(Cc3cccc(C(F)(F)F)c3)c12)c1ccc(C(=O)O)cc1]

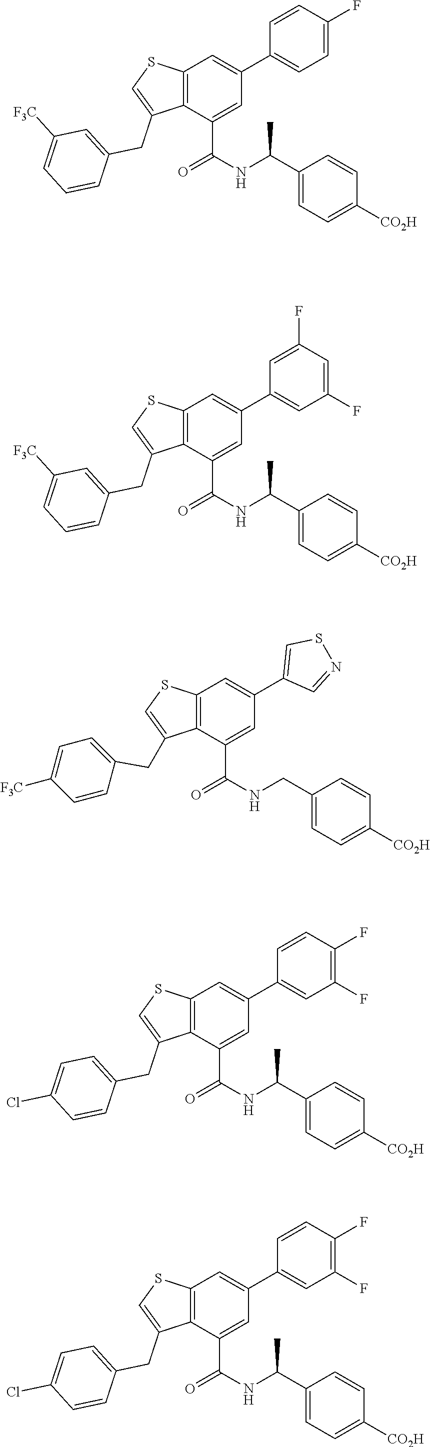 [SMILES: C[C@H](NC(=O)c1cc(-c2ccc(F)cc2)cc2scc(Cc3cccc(C(F)(F)F)c3)c12)c1ccc(C(=O)O)cc1 ; C[C@H](NC(=O)c1cc(-c2cc(F)cc(F)c2)cc2scc(Cc3cccc(C(F)(F)F)c3)c12)c1ccc(C(=O)O)cc1 ; O=C(O)c1ccc(CNC(=O)c2cc(-c3cnsc3)cc3scc(Cc4ccc(C(F)(F)F)cc4)c23)cc1 ; C[C@H](NC(=O)c1cc(-c2ccc(F)c(F)c2)cc2scc(Cc3ccc(Cl)cc3)c12)c1ccc(C(=O)O)cc1 ; C[C@H](NC(=O)c1cc(-c2ccc(F)c(F)c2)cc2scc(Cc3ccc(Cl)cc3)c12)c1ccc(C(=O)O)cc1]

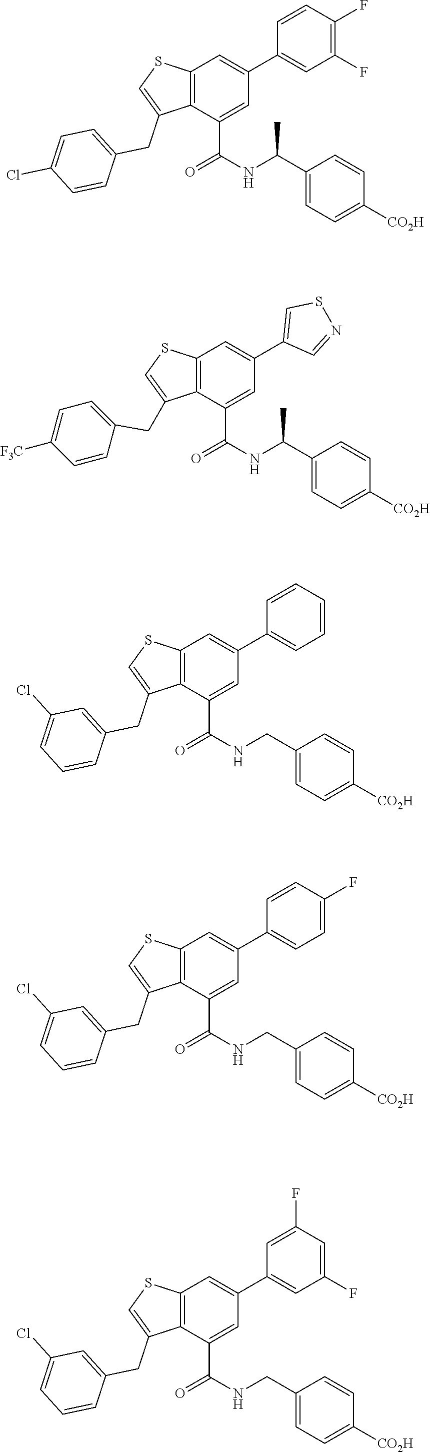 [SMILES: C[C@H](NC(=O)c1cc(-c2ccc(F)c(F)c2)cc2scc(Cc3ccc(Cl)cc3)c12)c1ccc(C(=O)O)cc1 ; C[C@H](NC(=O)c1cc(-c2cnsc2)cc2scc(Cc3ccc(C(F)(F)F)cc3)c12)c1ccc(C(=O)O)cc1 ; O=C(O)c1ccc(CNC(=O)c2cc(-c3ccccc3)cc3scc(Cc4cccc(Cl)c4)c23)cc1 ; O=C(O)c1ccc(CNC(=O)c2cc(-c3ccc(F)cc3)cc3scc(Cc4cccc(Cl)c4)c23)cc1 ; O=C(O)c1ccc(CNC(=O)c2cc(-c3cc(F)cc(F)c3)cc3scc(Cc4cccc(Cl)c4)c23)cc1]

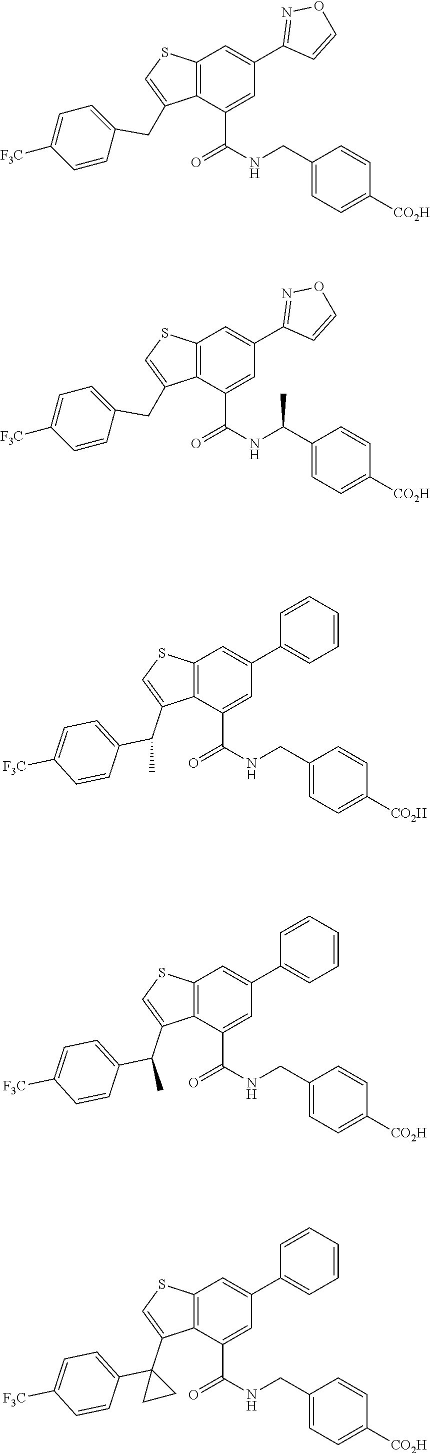 [SMILES: O=C(O)c1ccc(CNC(=O)c2cc(-c3ccon3)cc3scc(Cc4ccc(C(F)(F)F)cc4)c23)cc1 ; C[C@H](NC(=O)c1cc(-c2ccon2)cc2scc(Cc3ccc(C(F)(F)F)cc3)c12)c1ccc(C(=O)O)cc1 ; C[C@H](c1ccc(C(F)(F)F)cc1)c1csc2cc(-c3ccccc3)cc(C(=O)NCc3ccc(C(=O)O)cc3)c12 ; C[C@@H](c1ccc(C(F)(F)F)cc1)c1csc2cc(-c3ccccc3)cc(C(=O)NCc3ccc(C(=O)O)cc3)c12 ; O=C(O)c1ccc(CNC(=O)c2cc(-c3ccccc3)cc3scc(C4(c5ccc(C(F)(F)F)cc5)CC4)c23)cc1]

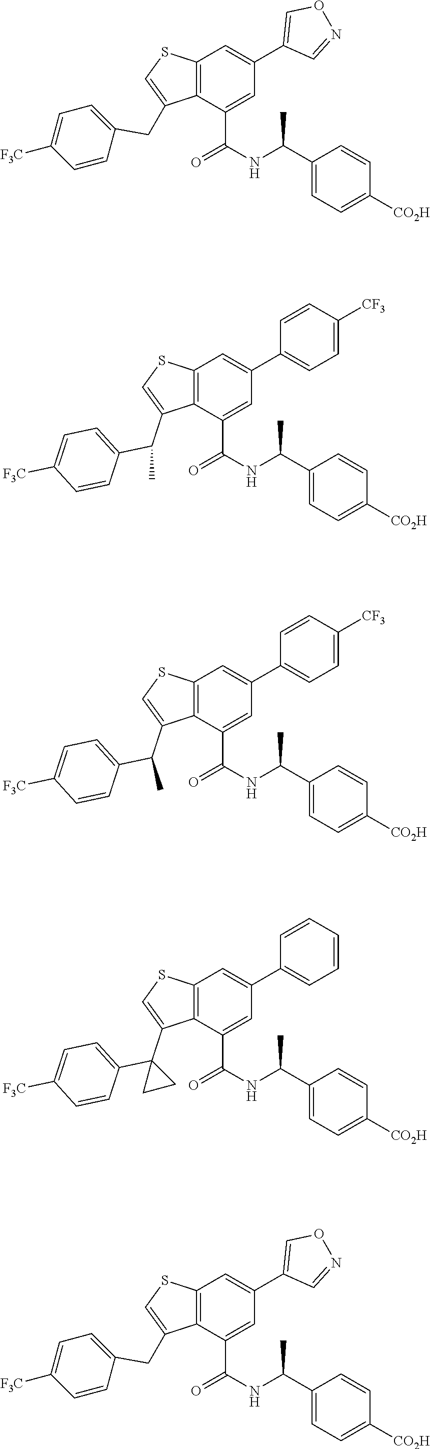 [SMILES: C[C@H](NC(=O)c1cc(-c2cnoc2)cc2scc(Cc3ccc(C(F)(F)F)cc3)c12)c1ccc(C(=O)O)cc1 ; C[C@H](c1ccc(C(F)(F)F)cc1)c1csc2cc(-c3ccc(C(F)(F)F)cc3)cc(C(=O)N[C@@H](C)c3ccc(C(=O)O)cc3)c12 ; C[C@@H](c1ccc(C(F)(F)F)cc1)c1csc2cc(-c3ccc(C(F)(F)F)cc3)cc(C(=O)N[C@@H](C)c3ccc(C(=O)O)cc3)c12 ; C[C@H](NC(=O)c1cc(-c2ccccc2)cc2scc(C3(c4ccc(C(F)(F)F)cc4)CC3)c12)c1ccc(C(=O)O)cc1 ; C[C@H](NC(=O)c1cc(-c2cnoc2)cc2scc(Cc3ccc(C(F)(F)F)cc3)c12)c1ccc(C(=O)O)cc1]

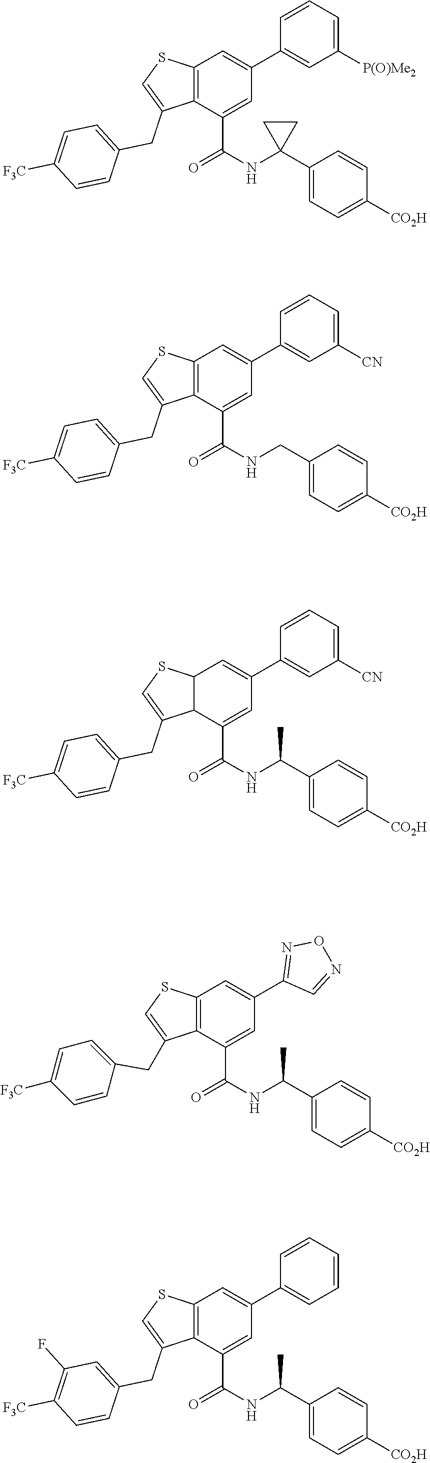

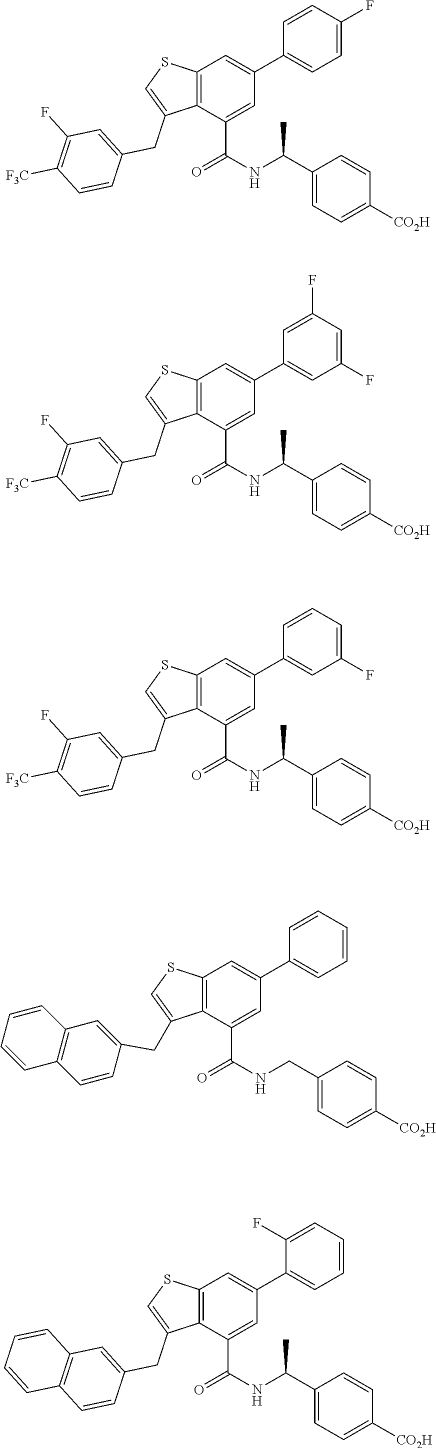 [SMILES: C[C@H](NC(=O)c1cc(-c2ccc(F)cc2)cc2scc(Cc3ccc(C(F)(F)F)c(F)c3)c12)c1ccc(C(=O)O)cc1 ; C[C@H](NC(=O)c1cc(-c2cc(F)cc(F)c2)cc2scc(Cc3ccc(C(F)(F)F)c(F)c3)c12)c1ccc(C(=O)O)cc1 ; C[C@H](NC(=O)c1cc(-c2cccc(F)c2)cc2scc(Cc3ccc(C(F)(F)F)c(F)c3)c12)c1ccc(C(=O)O)cc1 ; O=C(O)c1ccc(CNC(=O)c2cc(-c3ccccc3)cc3scc(Cc4ccc5ccccc5c4)c23)cc1 ; C[C@H](NC(=O)c1cc(-c2ccccc2F)cc2scc(Cc3ccc4ccccc4c3)c12)c1ccc(C(=O)O)cc1]

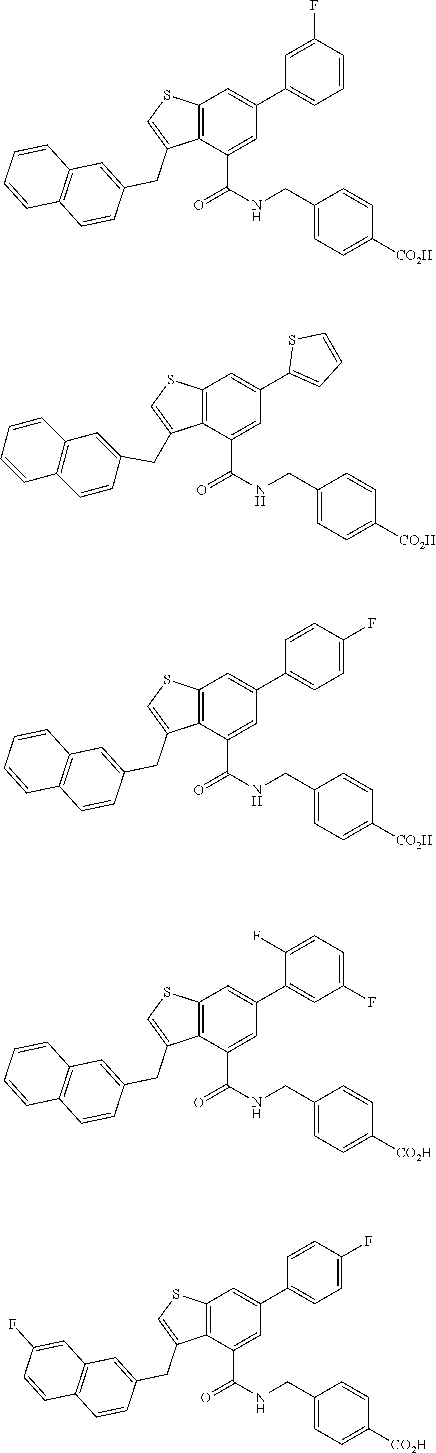 [SMILES: O=C(O)c1ccc(CNC(=O)c2cc(-c3cccc(F)c3)cc3scc(Cc4ccc5ccccc5c4)c23)cc1 ; O=C(O)c1ccc(CNC(=O)c2cc(-c3cccs3)cc3scc(Cc4ccc5ccccc5c4)c23)cc1 ; O=C(O)c1ccc(CNC(=O)c2cc(-c3ccc(F)cc3)cc3scc(Cc4ccc5ccccc5c4)c23)cc1 ; O=C(O)c1ccc(CNC(=O)c2cc(-c3cc(F)ccc3F)cc3scc(Cc4ccc5ccccc5c4)c23)cc1 ; O=C(O)c1ccc(CNC(=O)c2cc(-c3ccc(F)cc3)cc3scc(Cc4ccc5ccc(F)cc5c4)c23)cc1]

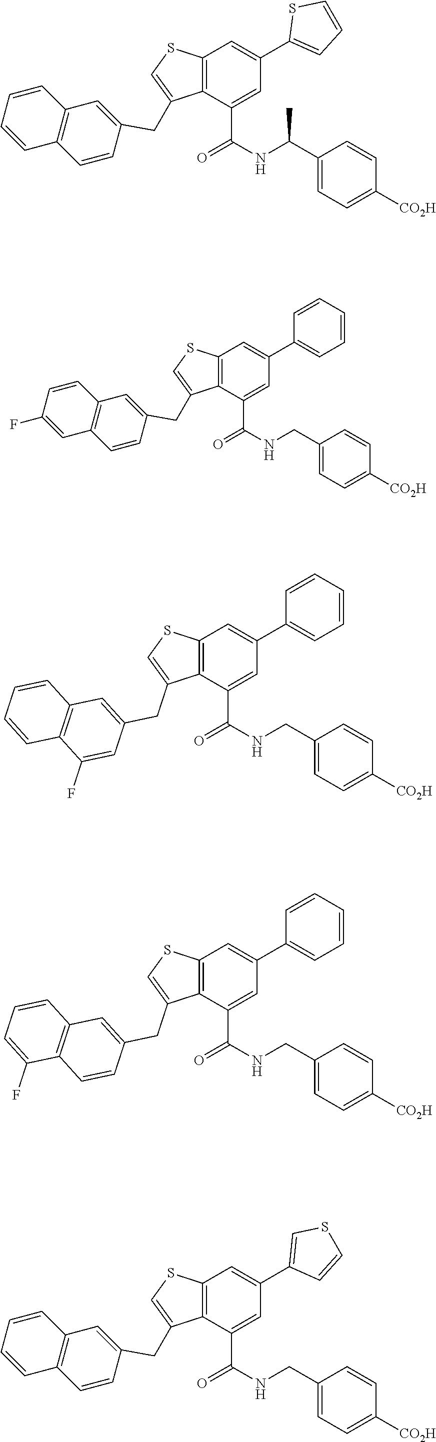 [SMILES: C[C@H](NC(=O)c1cc(-c2cccs2)cc2scc(Cc3ccc4ccccc4c3)c12)c1ccc(C(=O)O)cc1 ; O=C(O)c1ccc(CNC(=O)c2cc(-c3ccccc3)cc3scc(Cc4ccc5cc(F)ccc5c4)c23)cc1 ; O=C(O)c1ccc(CNC(=O)c2cc(-c3ccccc3)cc3scc(Cc4cc(F)c5ccccc5c4)c23)cc1 ; O=C(O)c1ccc(CNC(=O)c2cc(-c3ccccc3)cc3scc(Cc4ccc5c(F)cccc5c4)c23)cc1 ; O=C(O)c1ccc(CNC(=O)c2cc(-c3ccsc3)cc3scc(Cc4ccc5ccccc5c4)c23)cc1]

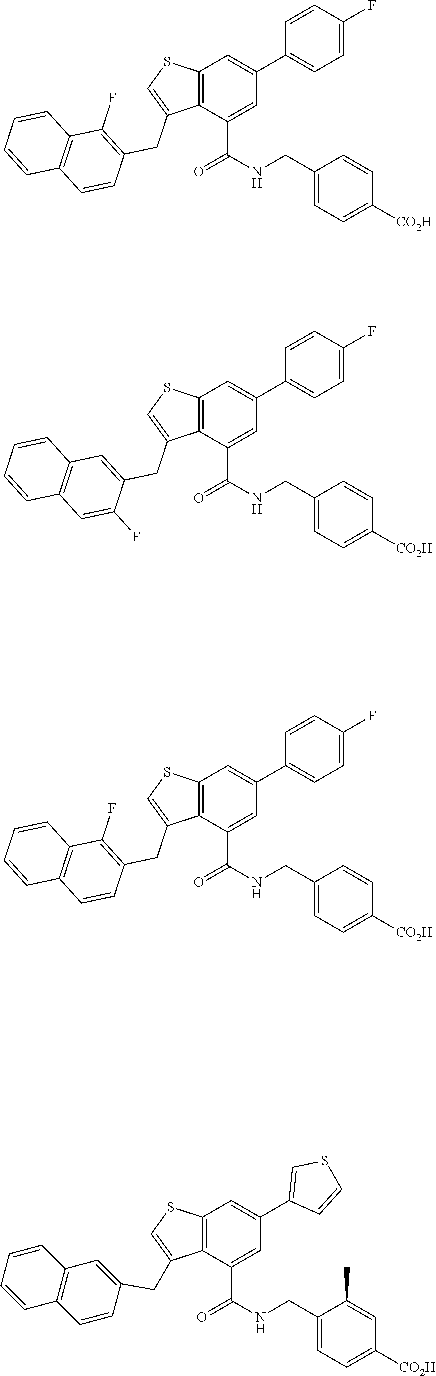

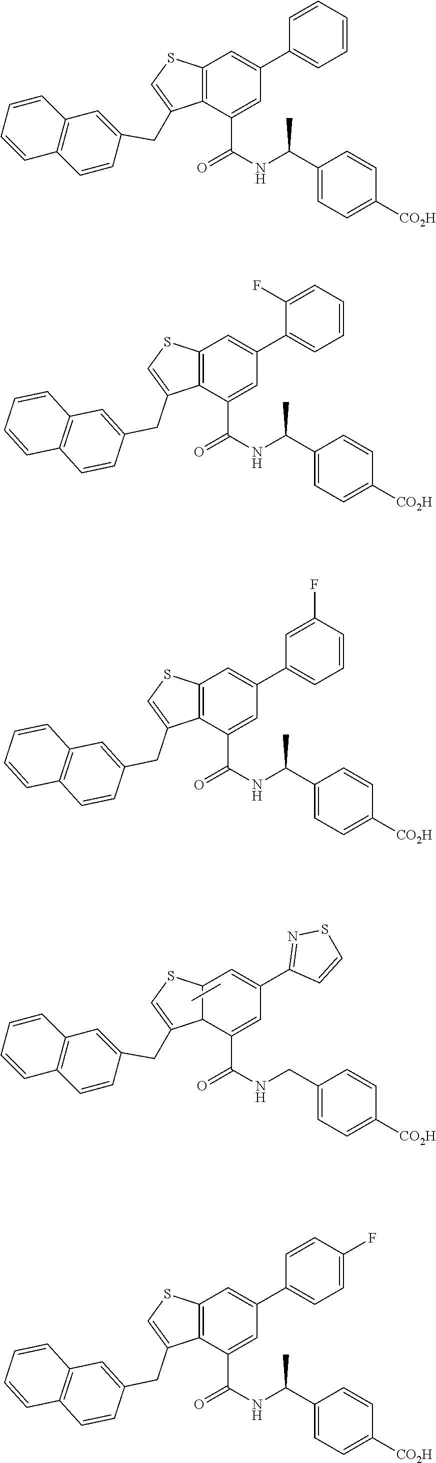

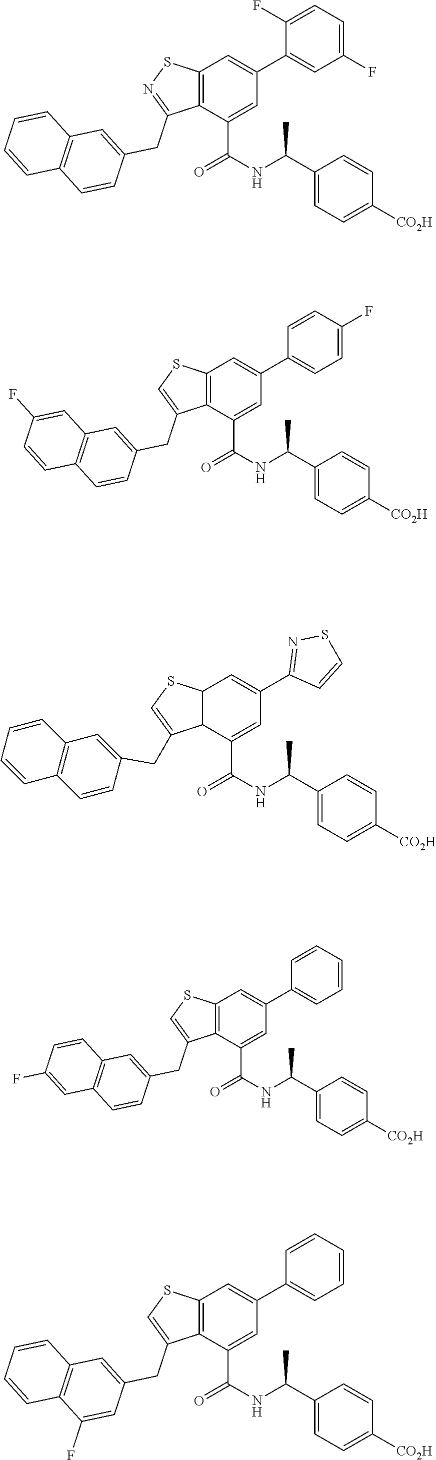 [SMILES: C[C@H](NC(=O)c1cc(-c2cc(F)ccc2F)cc2snc(Cc3ccc4ccccc4c3)c12)c1ccc(C(=O)O)cc1 ; C[C@H](NC(=O)c1cc(-c2ccc(F)cc2)cc2scc(Cc3ccc4ccc(F)cc4c3)c12)c1ccc(C(=O)O)cc1 ; C[C@H](NC(=O)C1=CC(c2ccsn2)=CC2SC=C(Cc3ccc4ccccc4c3)C12)c1ccc(C(=O)O)cc1 ; C[C@H](NC(=O)c1cc(-c2ccccc2)cc2scc(Cc3ccc4cc(F)ccc4c3)c12)c1ccc(C(=O)O)cc1 ; C[C@H](NC(=O)c1cc(-c2ccccc2)cc2scc(Cc3cc(F)c4ccccc4c3)c12)c1ccc(C(=O)O)cc1]

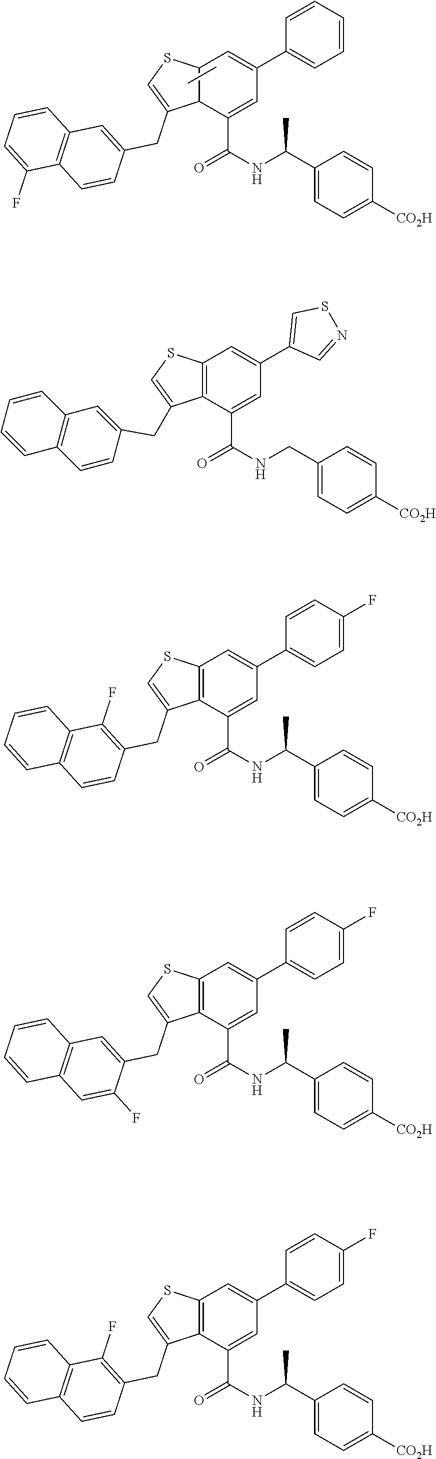

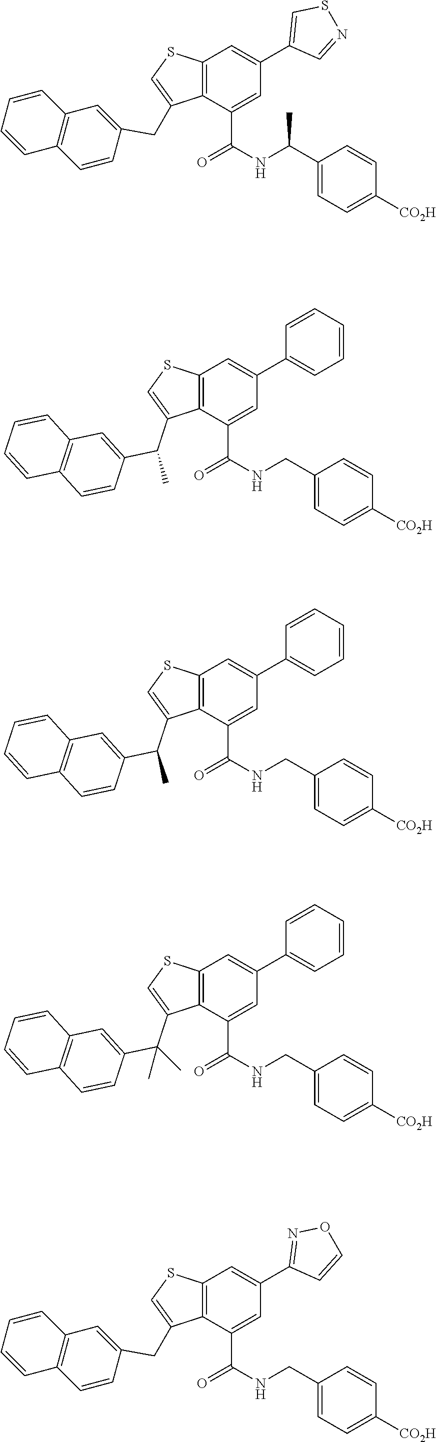 [SMILES: C[C@H](NC(=O)c1cc(-c2cnsc2)cc2scc(Cc3ccc4ccccc4c3)c12)c1ccc(C(=O)O)cc1 ; C[C@H](c1ccc2ccccc2c1)c1csc2cc(-c3ccccc3)cc(C(=O)NCc3ccc(C(=O)O)cc3)c12 ; C[C@@H](c1ccc2ccccc2c1)c1csc2cc(-c3ccccc3)cc(C(=O)NCc3ccc(C(=O)O)cc3)c12 ; CC(C)(c1ccc2ccccc2c1)c1csc2cc(-c3ccccc3)cc(C(=O)NCc3ccc(C(=O)O)cc3)c12 ; O=C(O)c1ccc(CNC(=O)c2cc(-c3ccon3)cc3scc(Cc4ccc5ccccc5c4)c23)cc1]

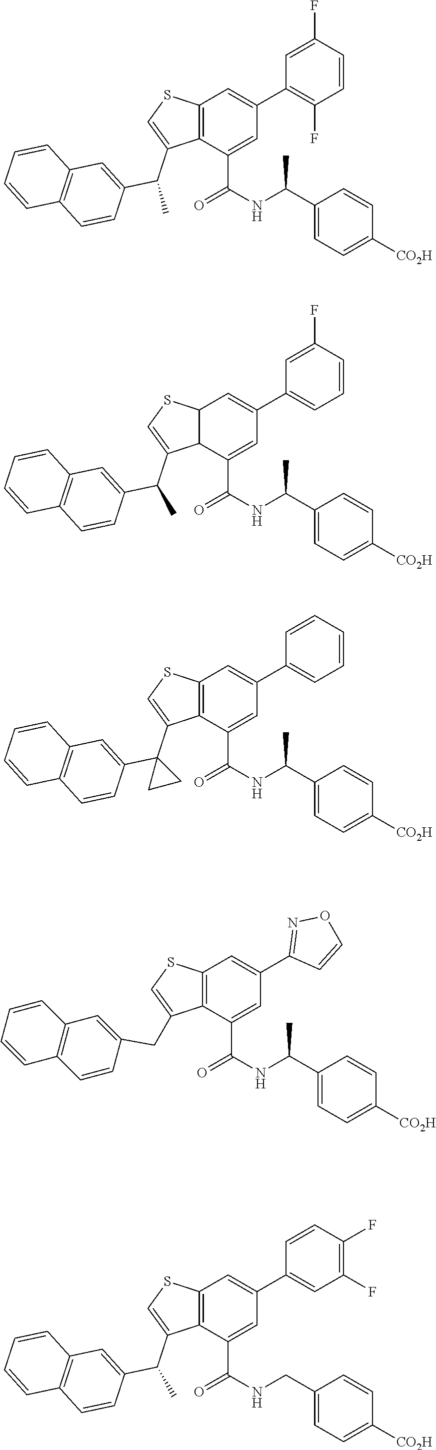 [SMILES: C[C@H](c1ccc2ccccc2c1)c1csc2cc(-c3cc(F)ccc3F)cc(C(=O)N[C@@H](C)c3ccc(C(=O)O)cc3)c12 ; C[C@H](NC(=O)C1=CC(c2cccc(F)c2)=CC2SC=C([C@@H](C)c3ccc4ccccc4c3)C12)c1ccc(C(=O)O)cc1 ; C[C@H](NC(=O)c1cc(-c2ccccc2)cc2scc(C3(c4ccc5ccccc5c4)CC3)c12)c1ccc(C(=O)O)cc1 ; C[C@H](NC(=O)c1cc(-c2ccon2)cc2scc(Cc3ccc4ccccc4c3)c12)c1ccc(C(=O)O)cc1 ; C[C@H](c1ccc2ccccc2c1)c1csc2cc(-c3ccc(F)c(F)c3)cc(C(=O)NCc3ccc(C(=O)O)cc3)c12]

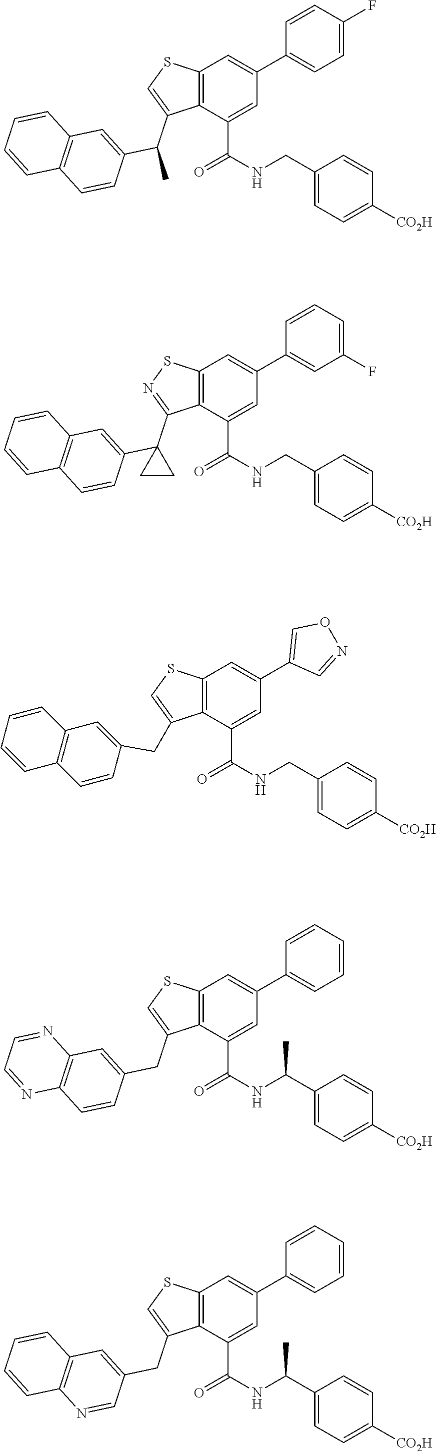 [SMILES: C[C@@H](c1ccc2ccccc2c1)c1csc2cc(-c3ccc(F)cc3)cc(C(=O)NCc3ccc(C(=O)O)cc3)c12 ; O=C(O)c1ccc(CNC(=O)c2cc(-c3cccc(F)c3)cc3snc(C4(c5ccc6ccccc6c5)CC4)c23)cc1 ; O=C(O)c1ccc(CNC(=O)c2cc(-c3cnoc3)cc3scc(Cc4ccc5ccccc5c4)c23)cc1 ; C[C@H](NC(=O)c1cc(-c2ccccc2)cc2scc(Cc3ccc4nccnc4c3)c12)c1ccc(C(=O)O)cc1 ; C[C@H](NC(=O)c1cc(-c2ccccc2)cc2scc(Cc3cnc4ccccc4c3)c12)c1ccc(C(=O)O)cc1]

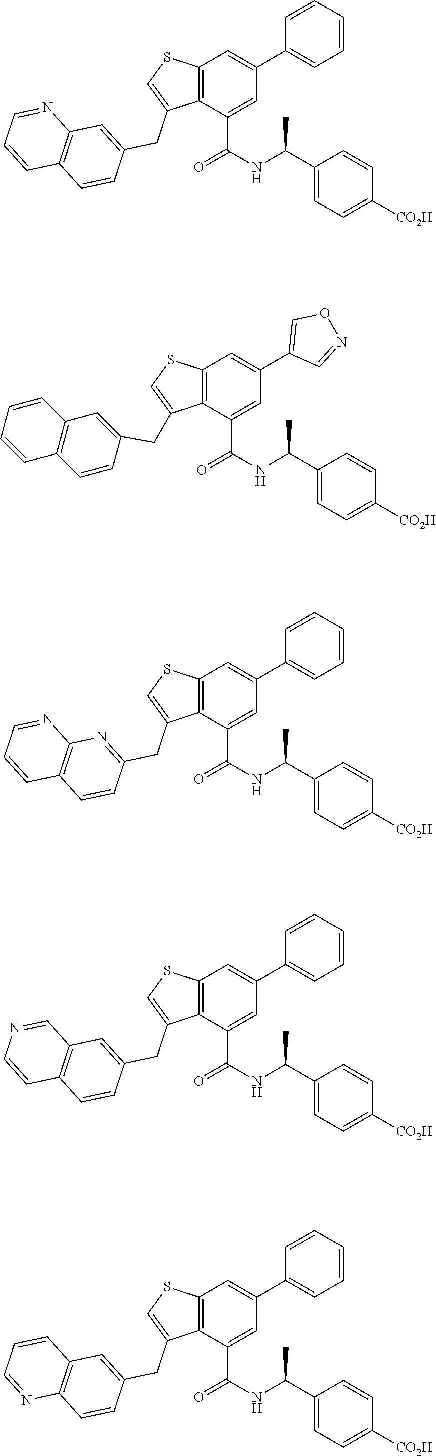 [SMILES: C[C@H](NC(=O)c1cc(-c2ccccc2)cc2scc(Cc3ccc4cccnc4c3)c12)c1ccc(C(=O)O)cc1 ; C[C@H](NC(=O)c1cc(-c2cnoc2)cc2scc(Cc3ccc4ccccc4c3)c12)c1ccc(C(=O)O)cc1 ; C[C@H](NC(=O)c1cc(-c2ccccc2)cc2scc(Cc3ccc4cccnc4n3)c12)c1ccc(C(=O)O)cc1 ; C[C@H](NC(=O)c1cc(-c2ccccc2)cc2scc(Cc3ccc4ccncc4c3)c12)c1ccc(C(=O)O)cc1 ; C[C@H](NC(=O)c1cc(-c2ccccc2)cc2scc(Cc3ccc4ncccc4c3)c12)c1ccc(C(=O)O)cc1]

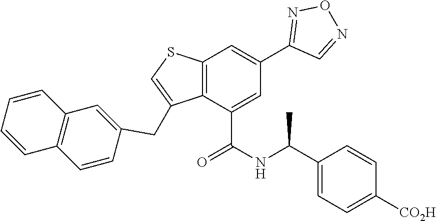 [SMILES: C[C@H](NC(=O)c1cc(-c2cnon2)cc2scc(Cc3ccc4ccccc4c3)c12)c1ccc(C(=O)O)cc1]

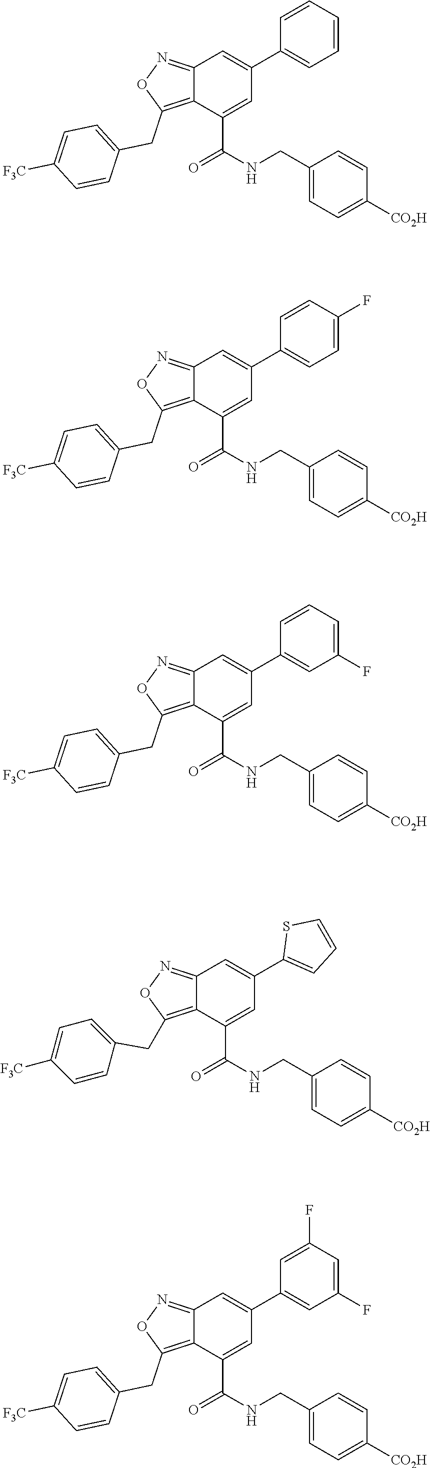 [SMILES: O=C(O)c1ccc(CNC(=O)c2cc(-c3ccccc3)cc3noc(Cc4ccc(C(F)(F)F)cc4)c23)cc1 ; O=C(O)c1ccc(CNC(=O)c2cc(-c3ccc(F)cc3)cc3noc(Cc4ccc(C(F)(F)F)cc4)c23)cc1 ; O=C(O)c1ccc(CNC(=O)c2cc(-c3cccc(F)c3)cc3noc(Cc4ccc(C(F)(F)F)cc4)c23)cc1 ; O=C(O)c1ccc(CNC(=O)c2cc(-c3cccs3)cc3noc(Cc4ccc(C(F)(F)F)cc4)c23)cc1 ; O=C(O)c1ccc(CNC(=O)c2cc(-c3cc(F)cc(F)c3)cc3noc(Cc4ccc(C(F)(F)F)cc4)c23)cc1]

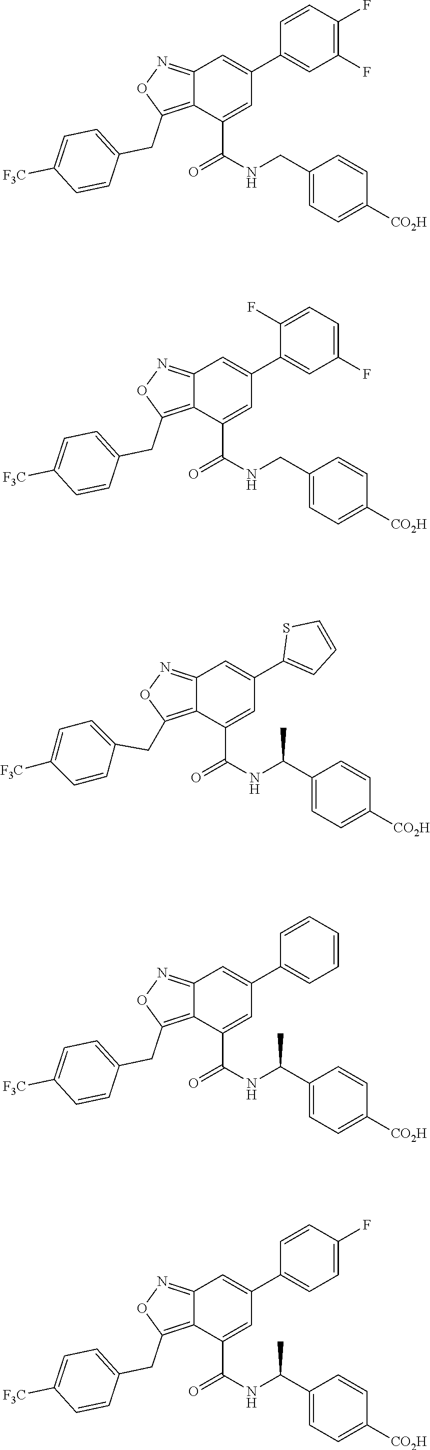 [SMILES: O=C(O)c1ccc(CNC(=O)c2cc(-c3ccc(F)c(F)c3)cc3noc(Cc4ccc(C(F)(F)F)cc4)c23)cc1 ; O=C(O)c1ccc(CNC(=O)c2cc(-c3cc(F)ccc3F)cc3noc(Cc4ccc(C(F)(F)F)cc4)c23)cc1 ; C[C@H](NC(=O)c1cc(-c2cccs2)cc2noc(Cc3ccc(C(F)(F)F)cc3)c12)c1ccc(C(=O)O)cc1 ; C[C@H](NC(=O)c1cc(-c2ccccc2)cc2noc(Cc3ccc(C(F)(F)F)cc3)c12)c1ccc(C(=O)O)cc1 ; C[C@H](NC(=O)c1cc(-c2ccc(F)cc2)cc2noc(Cc3ccc(C(F)(F)F)cc3)c12)c1ccc(C(=O)O)cc1]

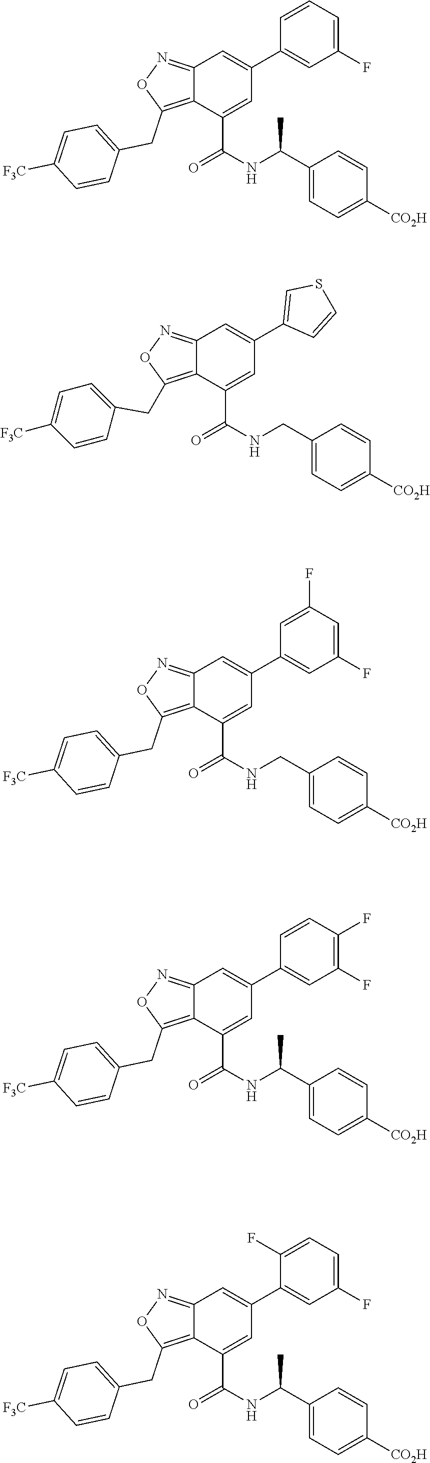 [SMILES: C[C@H](NC(=O)c1cc(-c2cccc(F)c2)cc2noc(Cc3ccc(C(F)(F)F)cc3)c12)c1ccc(C(=O)O)cc1 ; O=C(O)c1ccc(CNC(=O)c2cc(-c3ccsc3)cc3noc(Cc4ccc(C(F)(F)F)cc4)c23)cc1 ; O=C(O)c1ccc(CNC(=O)c2cc(-c3cc(F)cc(F)c3)cc3noc(Cc4ccc(C(F)(F)F)cc4)c23)cc1 ; C[C@H](NC(=O)c1cc(-c2ccc(F)c(F)c2)cc2noc(Cc3ccc(C(F)(F)F)cc3)c12)c1ccc(C(=O)O)cc1 ; C[C@H](NC(=O)c1cc(-c2cc(F)ccc2F)cc2noc(Cc3ccc(C(F)(F)F)cc3)c12)c1ccc(C(=O)O)cc1]

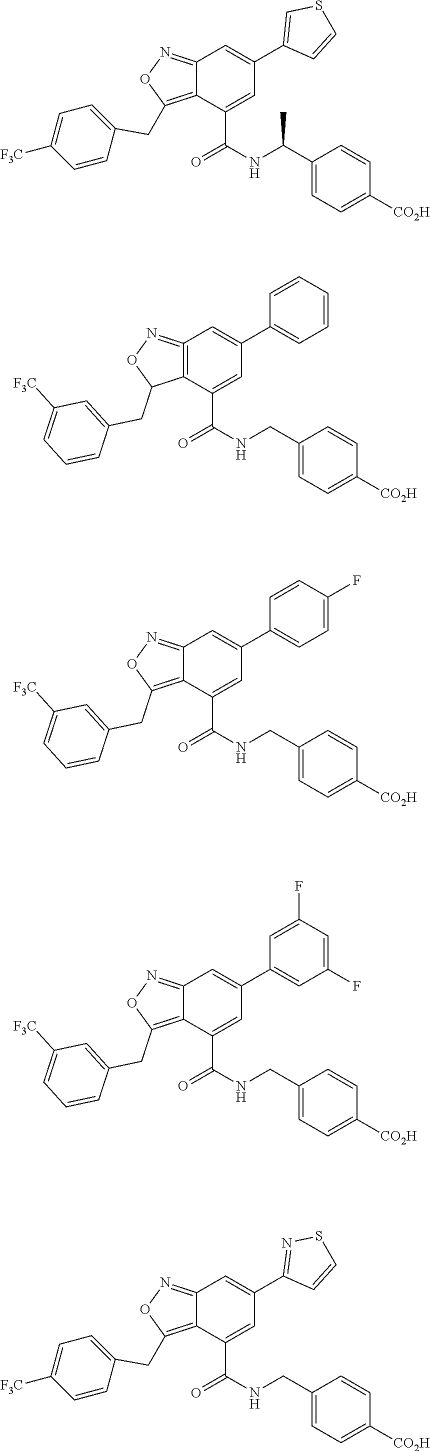

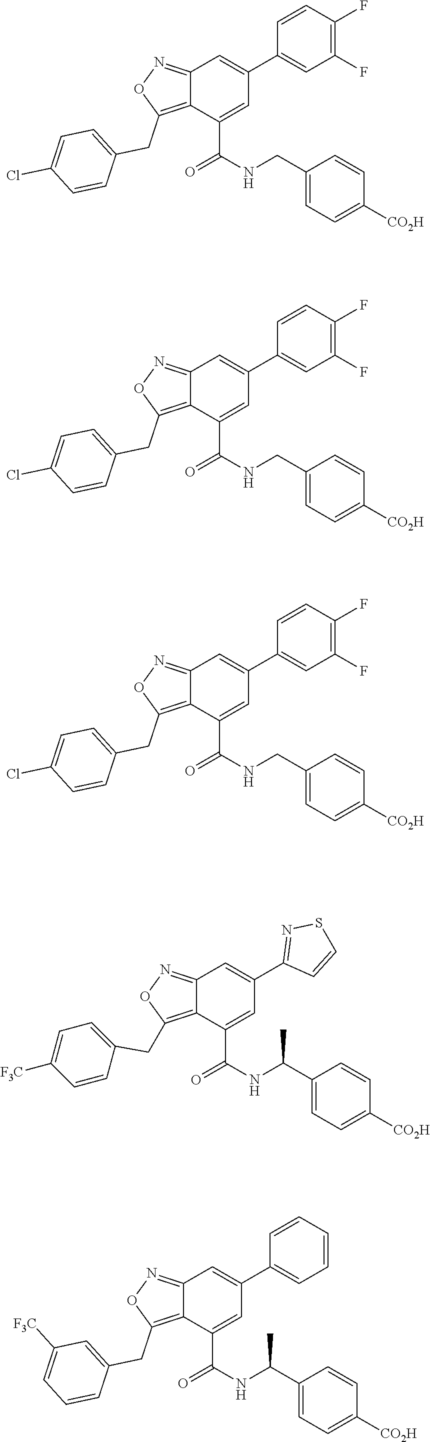 [SMILES: O=C(O)c1ccc(CNC(=O)c2cc(-c3ccc(F)c(F)c3)cc3noc(Cc4ccc(Cl)cc4)c23)cc1 ; O=C(O)c1ccc(CNC(=O)c2cc(-c3ccc(F)c(F)c3)cc3noc(Cc4ccc(Cl)cc4)c23)cc1 ; O=C(O)c1ccc(CNC(=O)c2cc(-c3ccc(F)c(F)c3)cc3noc(Cc4ccc(Cl)cc4)c23)cc1 ; C[C@H](NC(=O)c1cc(-c2ccsn2)cc2noc(Cc3ccc(C(F)(F)F)cc3)c12)c1ccc(C(=O)O)cc1 ; C[C@H](NC(=O)c1cc(-c2ccccc2)cc2noc(Cc3cccc(C(F)(F)F)c3)c12)c1ccc(C(=O)O)cc1]

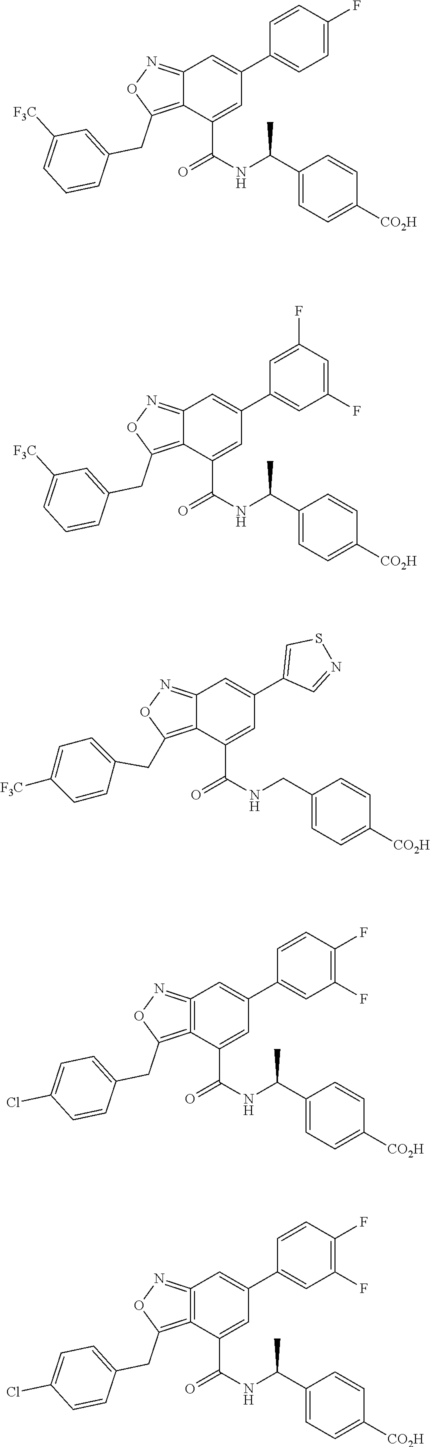

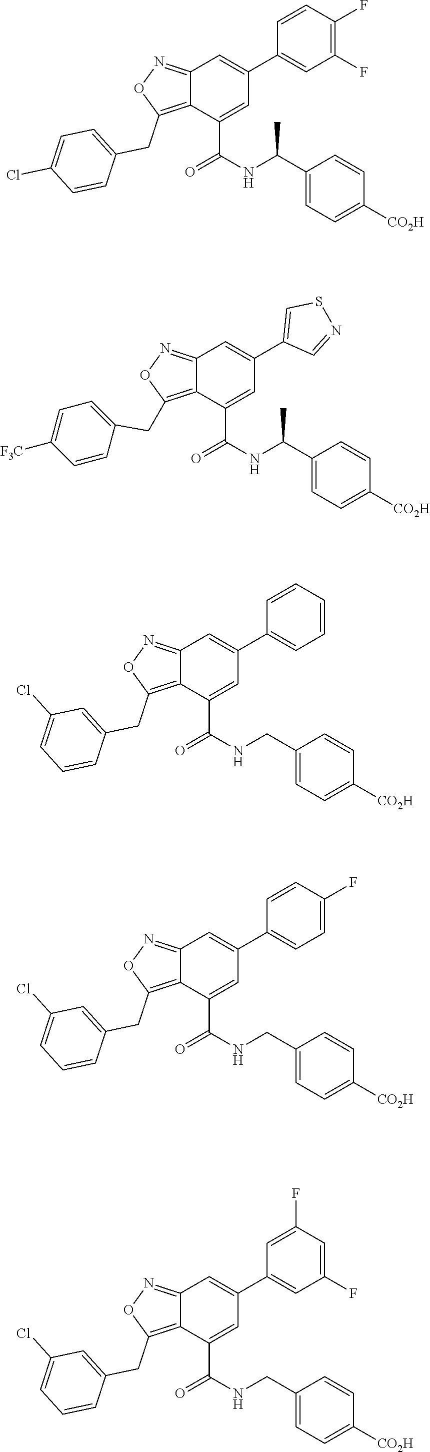 [SMILES: C[C@H](NC(=O)c1cc(-c2ccc(F)c(F)c2)cc2noc(Cc3ccc(Cl)cc3)c12)c1ccc(C(=O)O)cc1 ; C[C@H](NC(=O)c1cc(-c2cnsc2)cc2noc(Cc3ccc(C(F)(F)F)cc3)c12)c1ccc(C(=O)O)cc1 ; O=C(O)c1ccc(CNC(=O)c2cc(-c3ccccc3)cc3noc(Cc4cccc(Cl)c4)c23)cc1 ; O=C(O)c1ccc(CNC(=O)c2cc(-c3ccc(F)cc3)cc3noc(Cc4cccc(Cl)c4)c23)cc1 ; O=C(O)c1ccc(CNC(=O)c2cc(-c3cc(F)cc(F)c3)cc3noc(Cc4cccc(Cl)c4)c23)cc1]

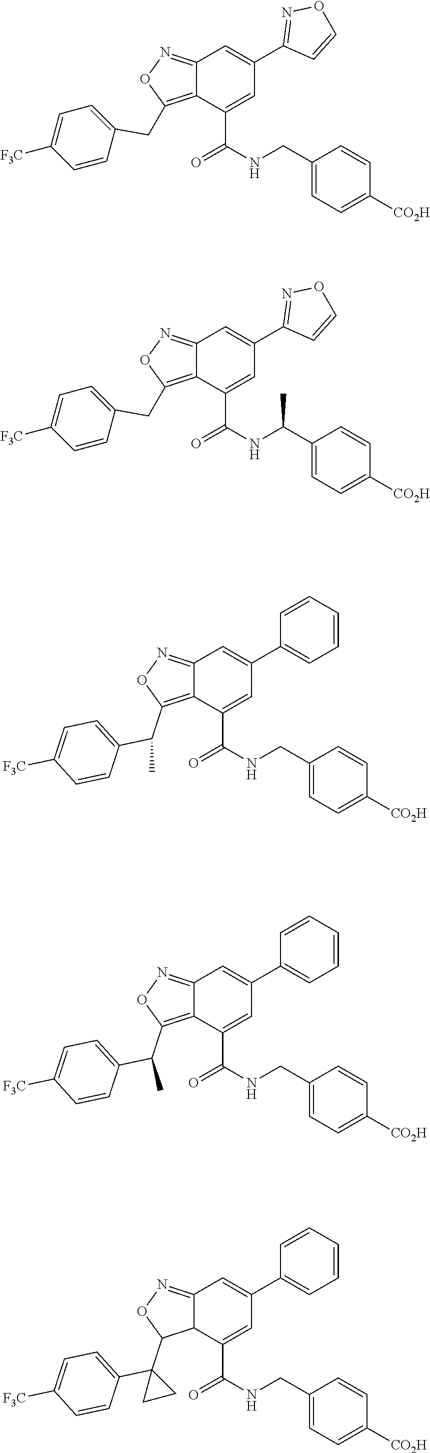 [SMILES: O=C(O)c1ccc(CNC(=O)c2cc(-c3ccon3)cc3noc(Cc4ccc(C(F)(F)F)cc4)c23)cc1 ; C[C@H](NC(=O)c1cc(-c2ccon2)cc2noc(Cc3ccc(C(F)(F)F)cc3)c12)c1ccc(C(=O)O)cc1 ; C[C@H](c1ccc(C(F)(F)F)cc1)c1onc2cc(-c3ccccc3)cc(C(=O)NCc3ccc(C(=O)O)cc3)c12 ; C[C@@H](c1ccc(C(F)(F)F)cc1)c1onc2cc(-c3ccccc3)cc(C(=O)NCc3ccc(C(=O)O)cc3)c12 ; O=C(NCc1ccc(C(=O)O)cc1)C1=CC(c2ccccc2)=CC2=NOC(C3(c4ccc(C(F)(F)F)cc4)CC3)C12]

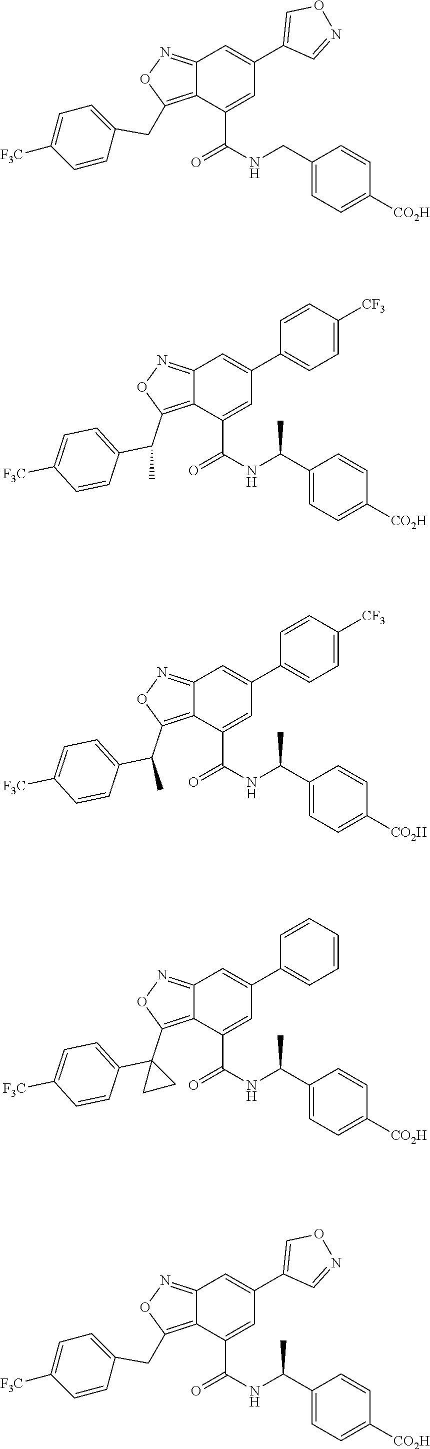 [SMILES: O=C(O)c1ccc(CNC(=O)c2cc(-c3cnoc3)cc3noc(Cc4ccc(C(F)(F)F)cc4)c23)cc1 ; C[C@H](c1ccc(C(F)(F)F)cc1)c1onc2cc(-c3ccc(C(F)(F)F)cc3)cc(C(=O)N[C@@H](C)c3ccc(C(=O)O)cc3)c12 ; C[C@@H](c1ccc(C(F)(F)F)cc1)c1onc2cc(-c3ccc(C(F)(F)F)cc3)cc(C(=O)N[C@@H](C)c3ccc(C(=O)O)cc3)c12 ; C[C@H](NC(=O)c1cc(-c2ccccc2)cc2noc(C3(c4ccc(C(F)(F)F)cc4)CC3)c12)c1ccc(C(=O)O)cc1 ; C[C@H](NC(=O)c1cc(-c2cnoc2)cc2noc(Cc3ccc(C(F)(F)F)cc3)c12)c1ccc(C(=O)O)cc1]

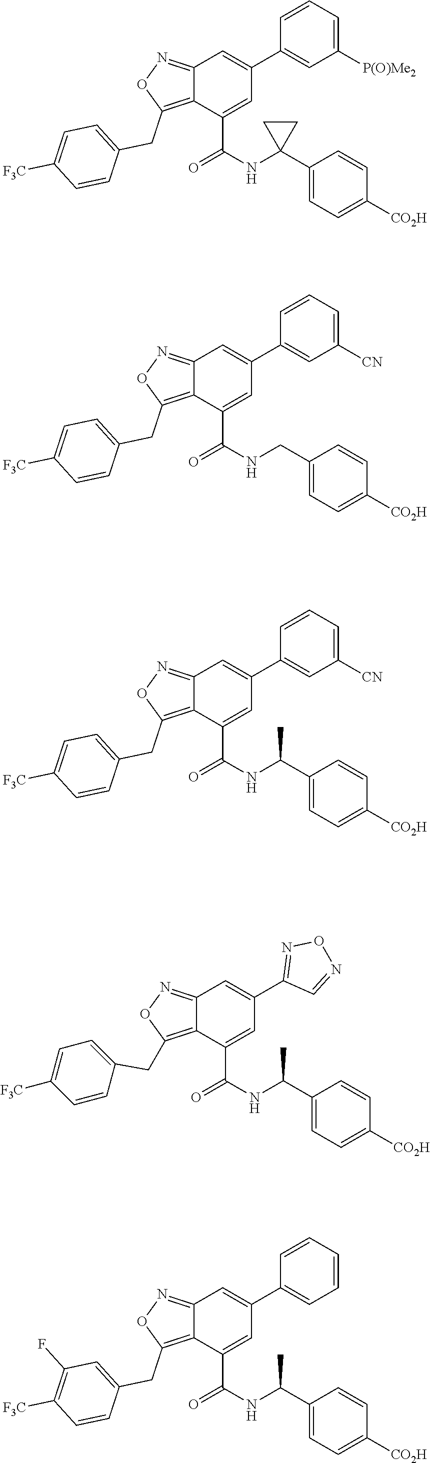

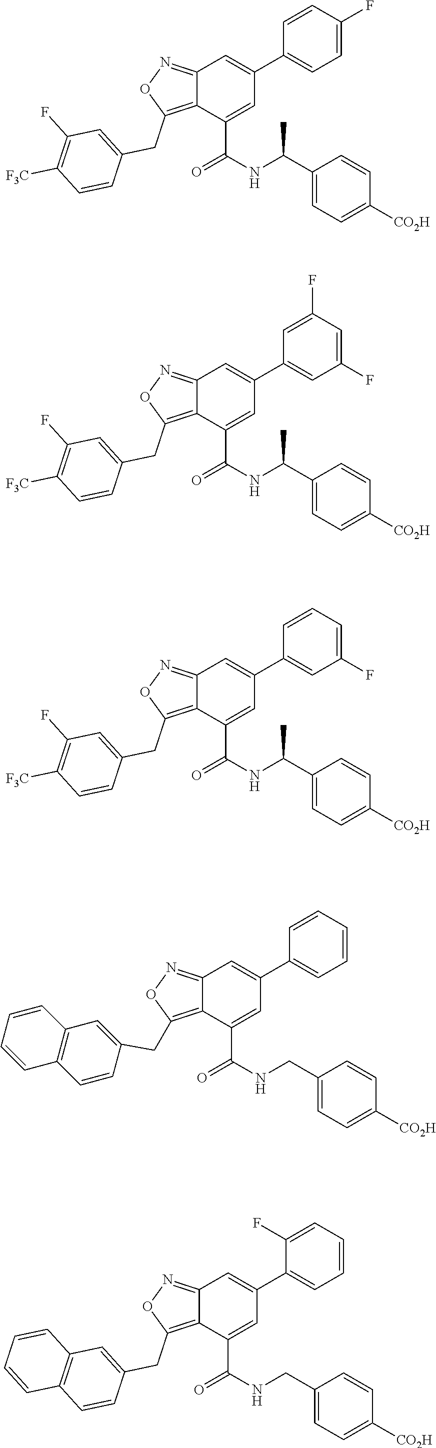 [SMILES: C[C@H](NC(=O)c1cc(-c2ccc(F)cc2)cc2noc(Cc3ccc(C(F)(F)F)c(F)c3)c12)c1ccc(C(=O)O)cc1 ; C[C@H](NC(=O)c1cc(-c2cc(F)cc(F)c2)cc2noc(Cc3ccc(C(F)(F)F)c(F)c3)c12)c1ccc(C(=O)O)cc1 ; C[C@H](NC(=O)c1cc(-c2cccc(F)c2)cc2noc(Cc3ccc(C(F)(F)F)c(F)c3)c12)c1ccc(C(=O)O)cc1 ; O=C(O)c1ccc(CNC(=O)c2cc(-c3ccccc3)cc3noc(Cc4ccc5ccccc5c4)c23)cc1 ; O=C(O)c1ccc(CNC(=O)c2cc(-c3ccccc3F)cc3noc(Cc4ccc5ccccc5c4)c23)cc1]

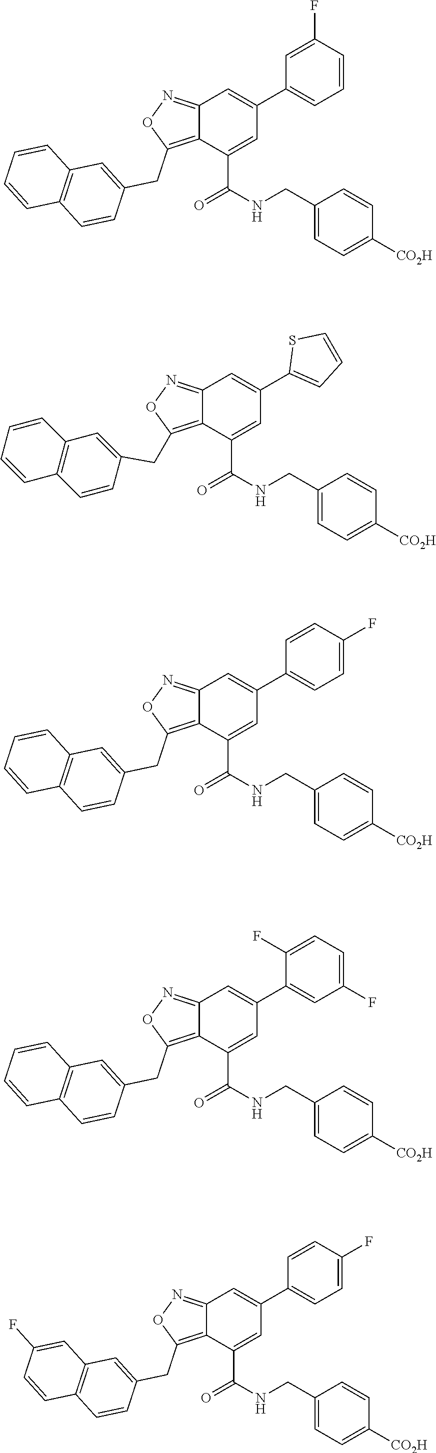 [SMILES: O=C(O)c1ccc(CNC(=O)c2cc(-c3cccc(F)c3)cc3noc(Cc4ccc5ccccc5c4)c23)cc1 ; O=C(O)c1ccc(CNC(=O)c2cc(-c3cccs3)cc3noc(Cc4ccc5ccccc5c4)c23)cc1 ; O=C(O)c1ccc(CNC(=O)c2cc(-c3ccc(F)cc3)cc3noc(Cc4ccc5ccccc5c4)c23)cc1 ; O=C(O)c1ccc(CNC(=O)c2cc(-c3cc(F)ccc3F)cc3noc(Cc4ccc5ccccc5c4)c23)cc1 ; O=C(O)c1ccc(CNC(=O)c2cc(-c3ccc(F)cc3)cc3noc(Cc4ccc5ccc(F)cc5c4)c23)cc1]

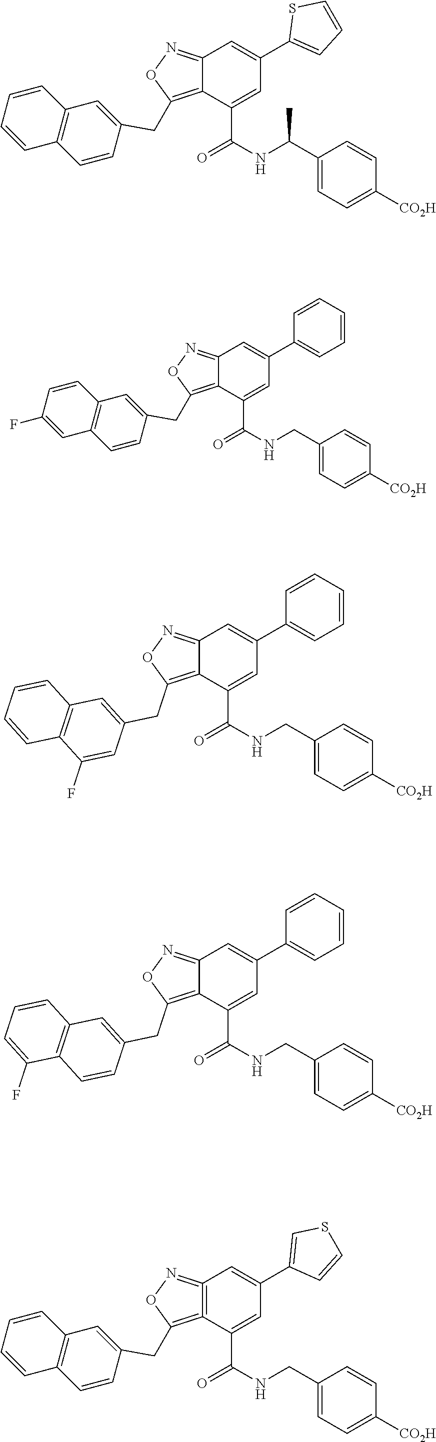

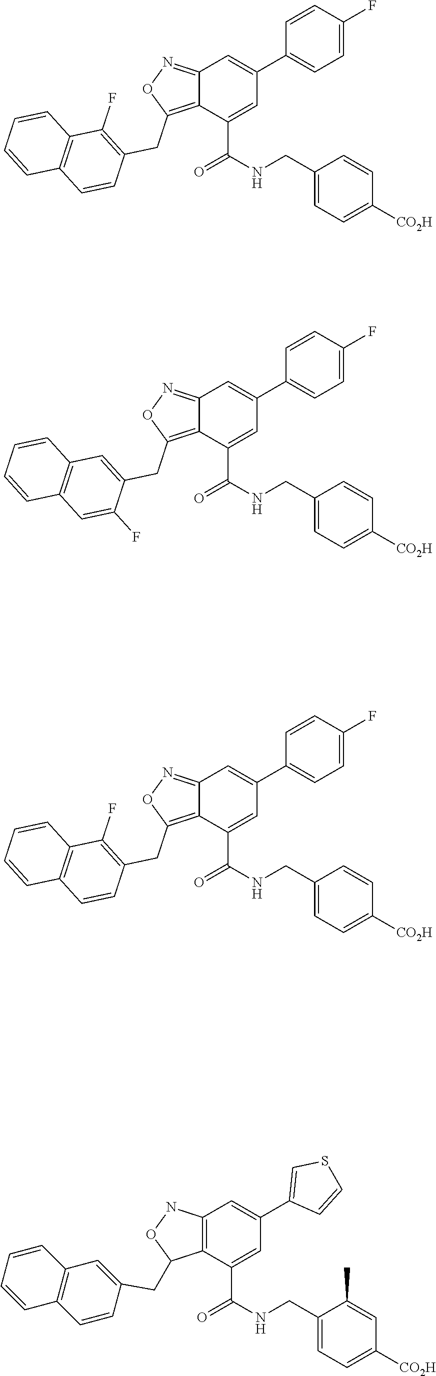

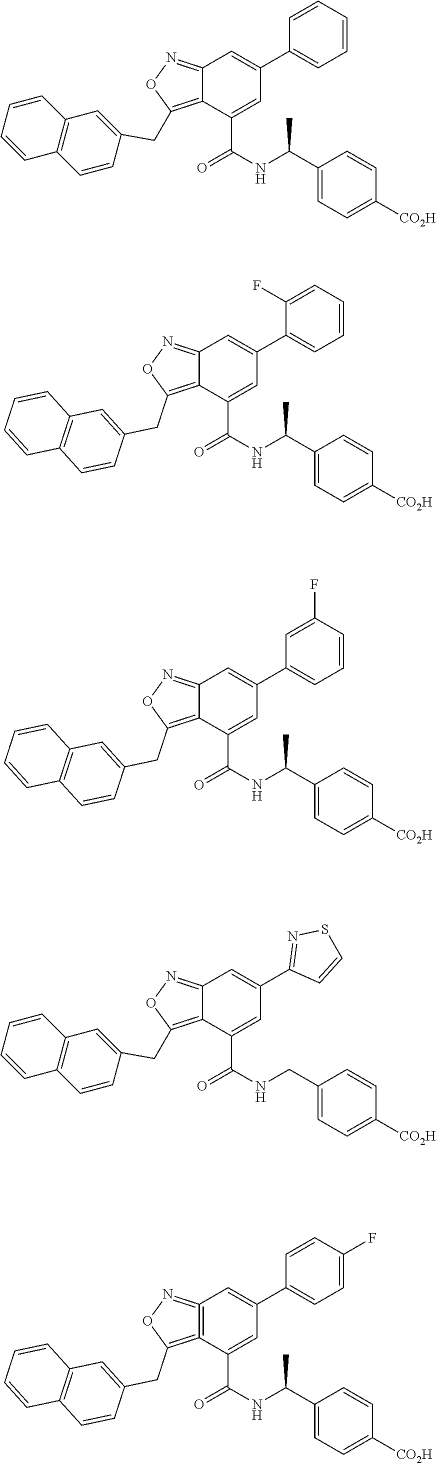

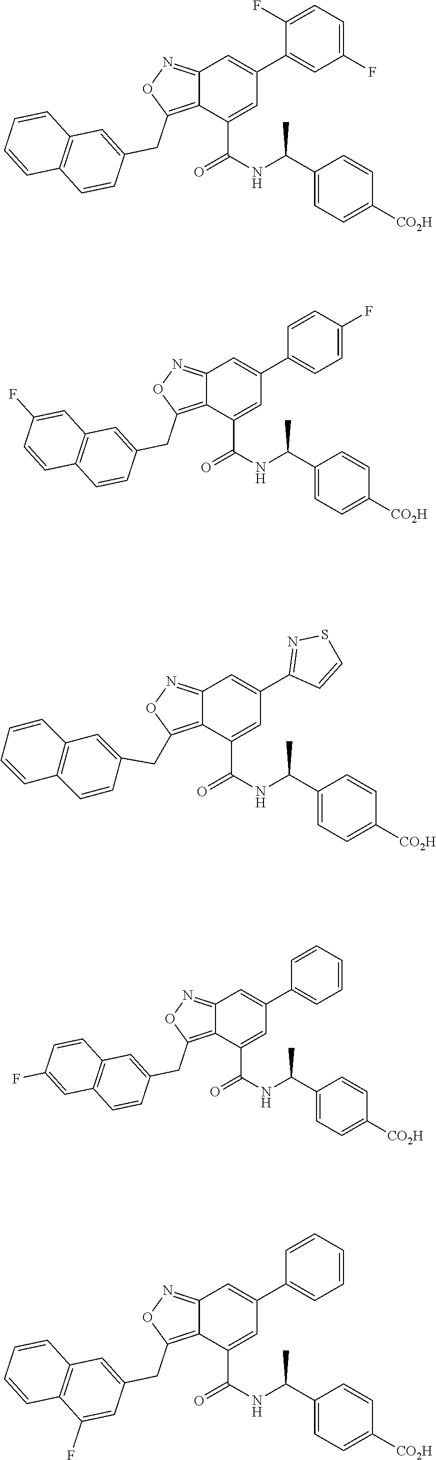 [SMILES: C[C@H](NC(=O)c1cc(-c2cc(F)ccc2F)cc2noc(Cc3ccc4ccccc4c3)c12)c1ccc(C(=O)O)cc1 ; C[C@H](NC(=O)c1cc(-c2ccc(F)cc2)cc2noc(Cc3ccc4ccc(F)cc4c3)c12)c1ccc(C(=O)O)cc1 ; C[C@H](NC(=O)c1cc(-c2ccsn2)cc2noc(Cc3ccc4ccccc4c3)c12)c1ccc(C(=O)O)cc1 ; C[C@H](NC(=O)c1cc(-c2ccccc2)cc2noc(Cc3ccc4cc(F)ccc4c3)c12)c1ccc(C(=O)O)cc1 ; C[C@H](NC(=O)c1cc(-c2ccccc2)cc2noc(Cc3cc(F)c4ccccc4c3)c12)c1ccc(C(=O)O)cc1]

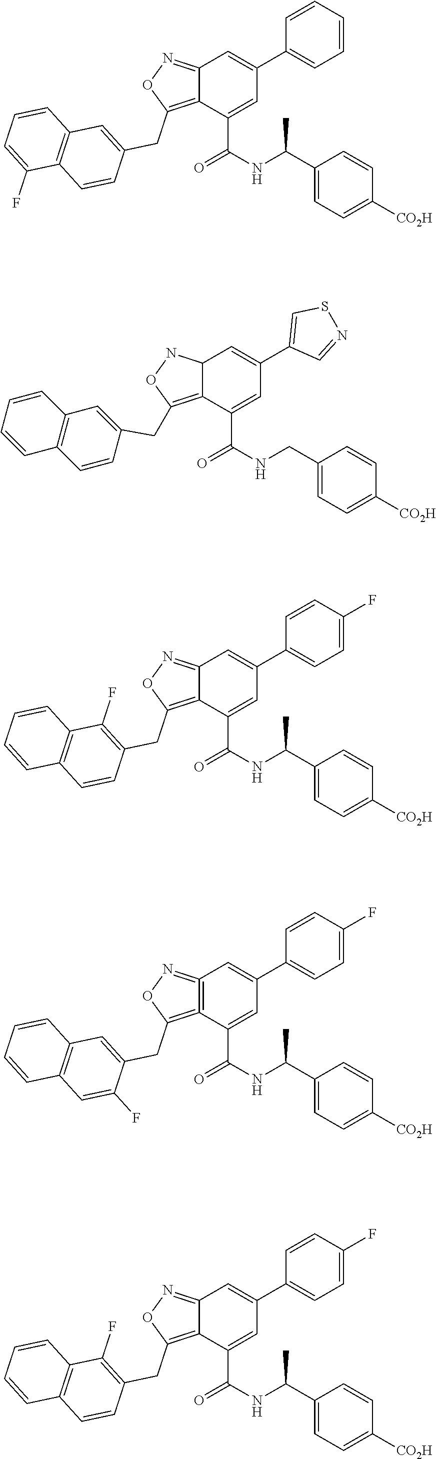

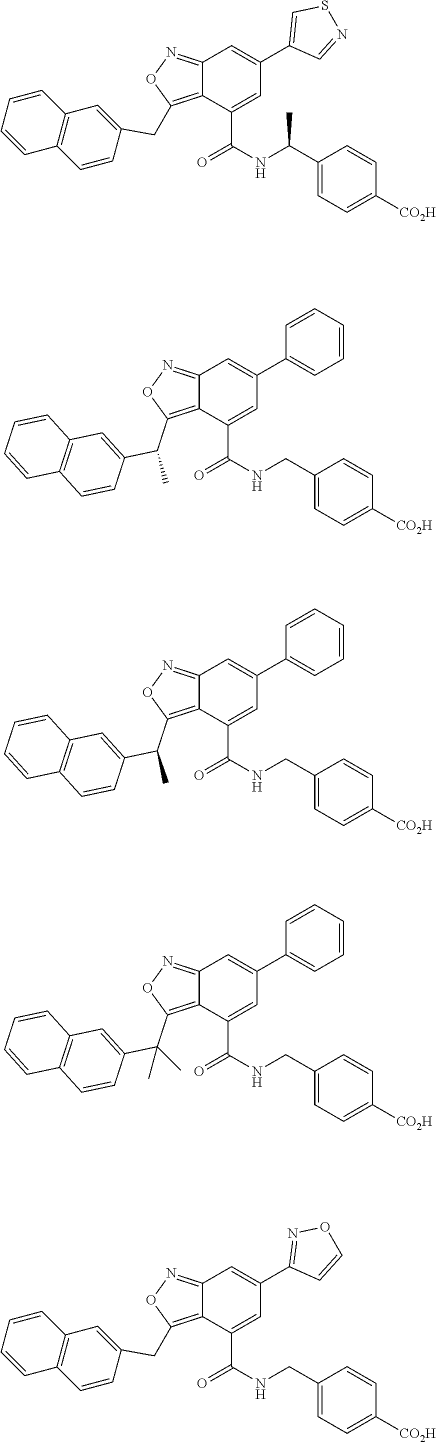 [SMILES: C[C@H](NC(=O)c1cc(-c2cnsc2)cc2noc(Cc3ccc4ccccc4c3)c12)c1ccc(C(=O)O)cc1 ; C[C@H](c1ccc2ccccc2c1)c1onc2cc(-c3ccccc3)cc(C(=O)NCc3ccc(C(=O)O)cc3)c12 ; C[C@@H](c1ccc2ccccc2c1)c1onc2cc(-c3ccccc3)cc(C(=O)NCc3ccc(C(=O)O)cc3)c12 ; CC(C)(c1ccc2ccccc2c1)c1onc2cc(-c3ccccc3)cc(C(=O)NCc3ccc(C(=O)O)cc3)c12 ; O=C(O)c1ccc(CNC(=O)c2cc(-c3ccon3)cc3noc(Cc4ccc5ccccc5c4)c23)cc1]

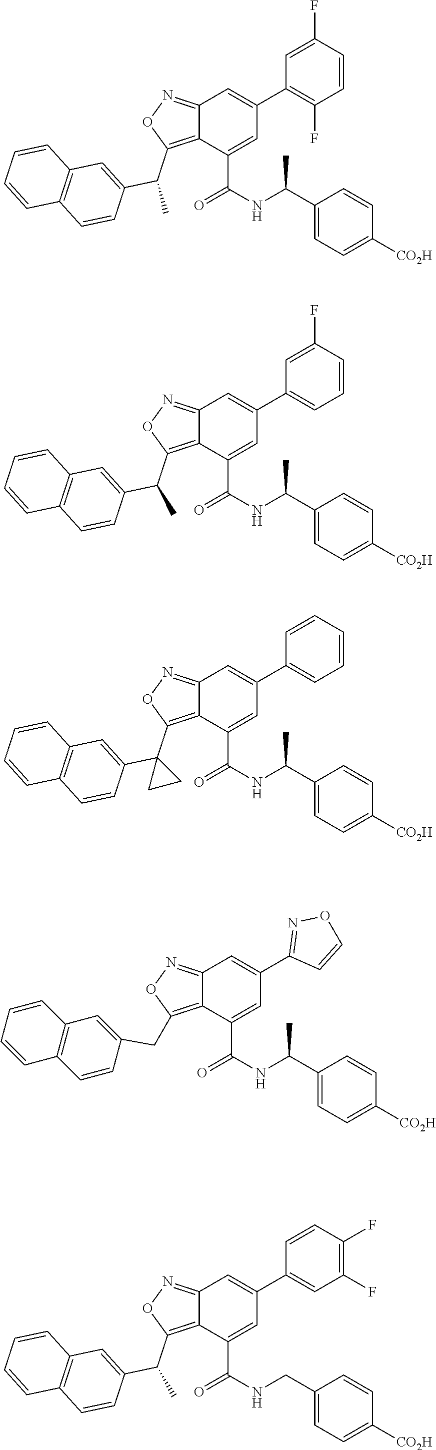 [SMILES: C[C@H](c1ccc2ccccc2c1)c1onc2cc(-c3cc(F)ccc3F)cc(C(=O)N[C@@H](C)c3ccc(C(=O)O)cc3)c12 ; C[C@@H](c1ccc2ccccc2c1)c1onc2cc(-c3cccc(F)c3)cc(C(=O)N[C@@H](C)c3ccc(C(=O)O)cc3)c12 ; C[C@H](NC(=O)c1cc(-c2ccccc2)cc2noc(C3(c4ccc5ccccc5c4)CC3)c12)c1ccc(C(=O)O)cc1 ; C[C@H](NC(=O)c1cc(-c2ccon2)cc2noc(Cc3ccc4ccccc4c3)c12)c1ccc(C(=O)O)cc1 ; C[C@H](c1ccc2ccccc2c1)c1onc2cc(-c3ccc(F)c(F)c3)cc(C(=O)NCc3ccc(C(=O)O)cc3)c12]

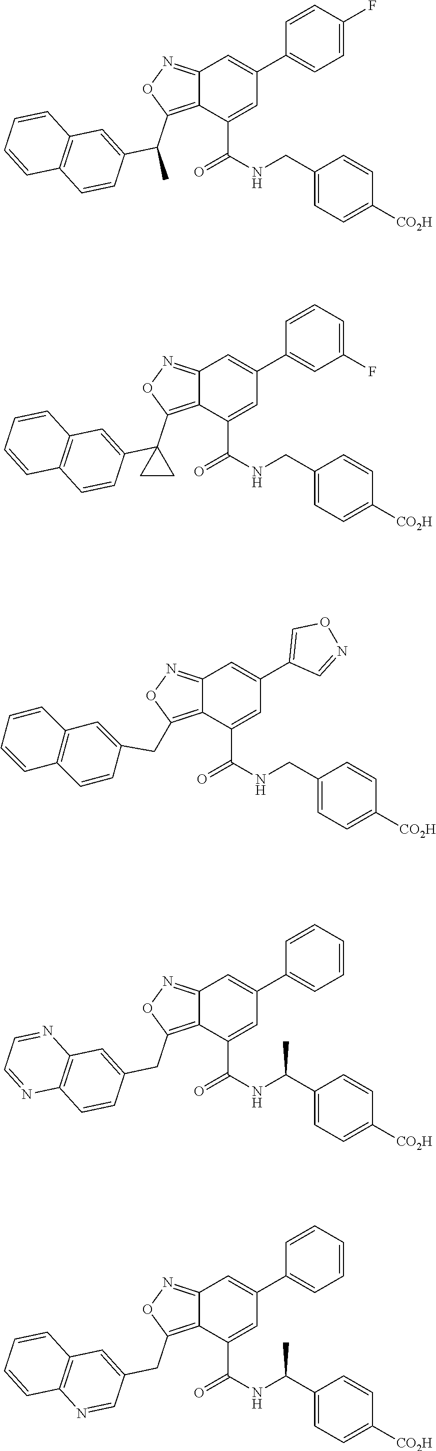 [SMILES: C[C@@H](c1ccc2ccccc2c1)c1onc2cc(-c3ccc(F)cc3)cc(C(=O)NCc3ccc(C(=O)O)cc3)c12 ; O=C(O)c1ccc(CNC(=O)c2cc(-c3cccc(F)c3)cc3noc(C4(c5ccc6ccccc6c5)CC4)c23)cc1 ; O=C(O)c1ccc(CNC(=O)c2cc(-c3cnoc3)cc3noc(Cc4ccc5ccccc5c4)c23)cc1 ; C[C@H](NC(=O)c1cc(-c2ccccc2)cc2noc(Cc3ccc4nccnc4c3)c12)c1ccc(C(=O)O)cc1 ; C[C@H](NC(=O)c1cc(-c2ccccc2)cc2noc(Cc3cnc4ccccc4c3)c12)c1ccc(C(=O)O)cc1]

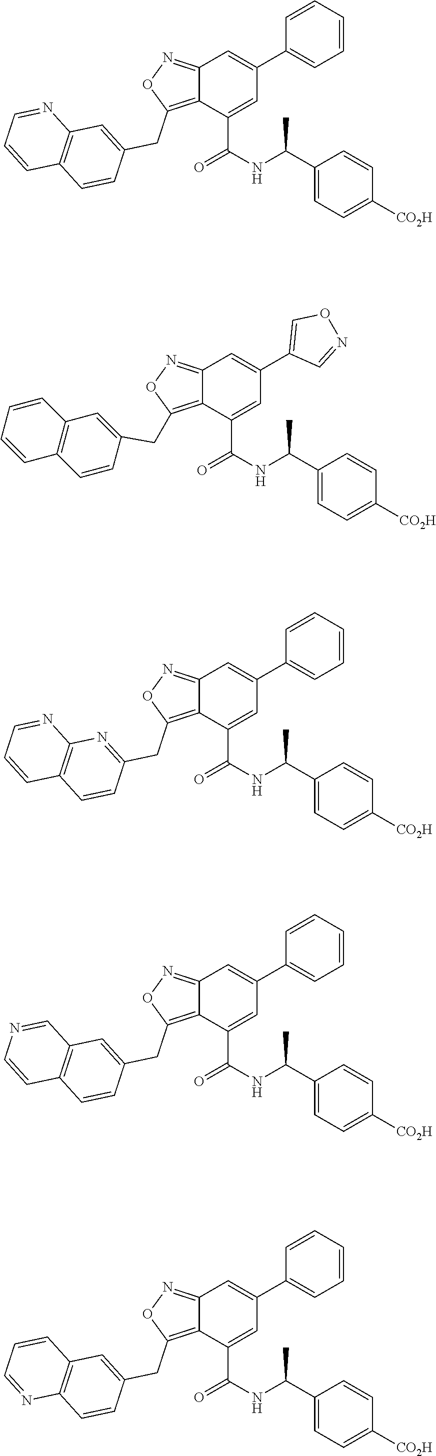 [SMILES: C[C@H](NC(=O)c1cc(-c2ccccc2)cc2noc(Cc3ccc4cccnc4c3)c12)c1ccc(C(=O)O)cc1 ; C[C@H](NC(=O)c1cc(-c2cnoc2)cc2noc(Cc3ccc4ccccc4c3)c12)c1ccc(C(=O)O)cc1 ; C[C@H](NC(=O)c1cc(-c2ccccc2)cc2noc(Cc3ccc4cccnc4n3)c12)c1ccc(C(=O)O)cc1 ; C[C@H](NC(=O)c1cc(-c2ccccc2)cc2noc(Cc3ccc4ccncc4c3)c12)c1ccc(C(=O)O)cc1 ; C[C@H](NC(=O)c1cc(-c2ccccc2)cc2noc(Cc3ccc4ncccc4c3)c12)c1ccc(C(=O)O)cc1]

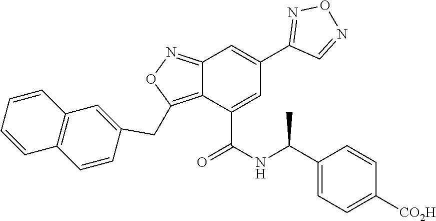 [SMILES: C[C@H](NC(=O)c1cc(-c2cnon2)cc2noc(Cc3ccc4ccccc4c3)c12)c1ccc(C(=O)O)cc1]

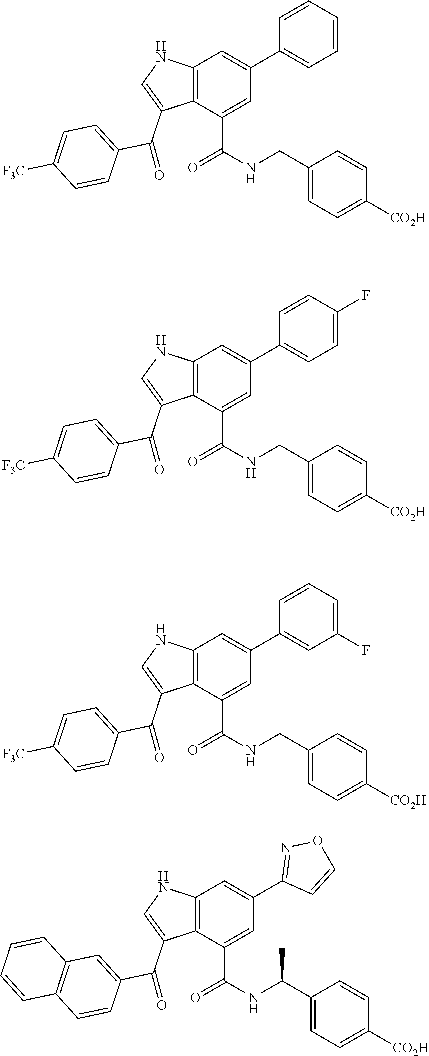

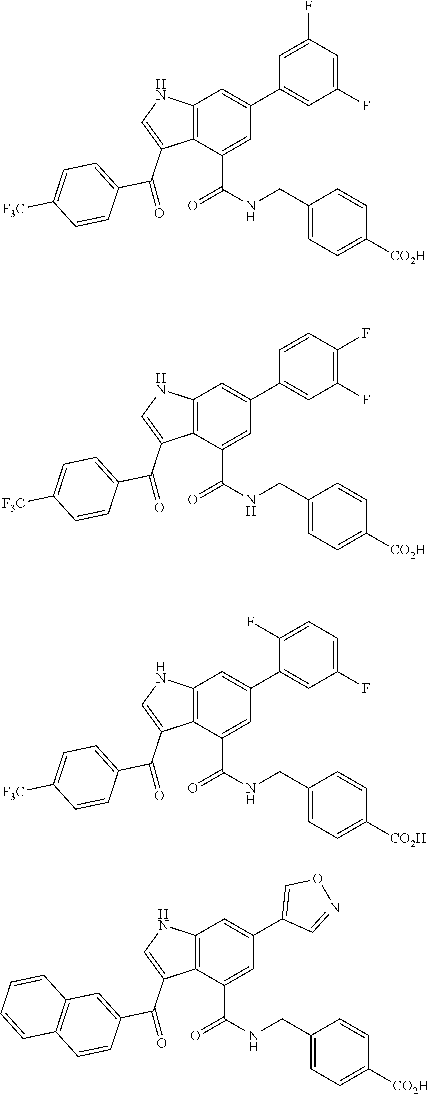

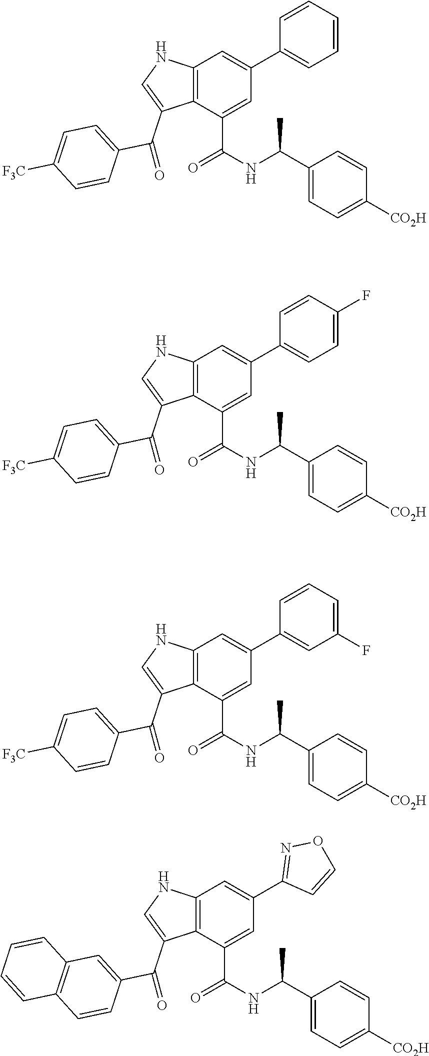

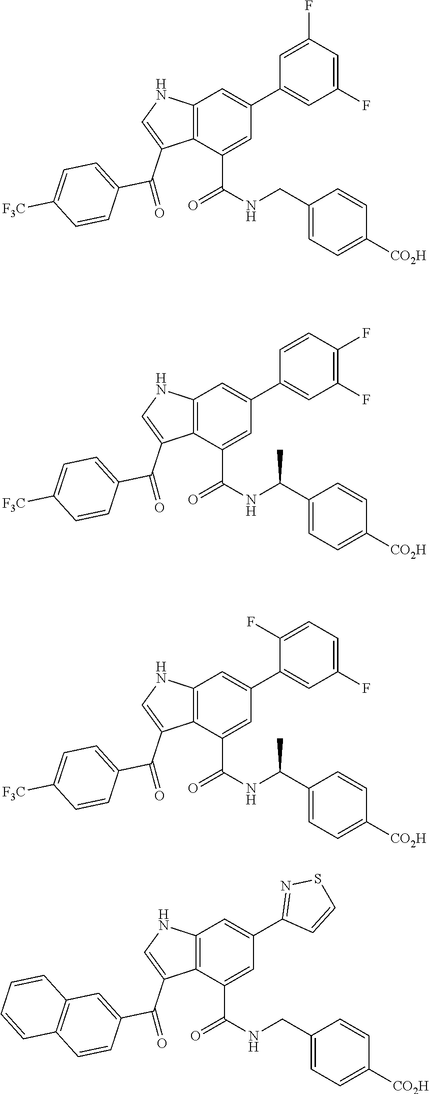

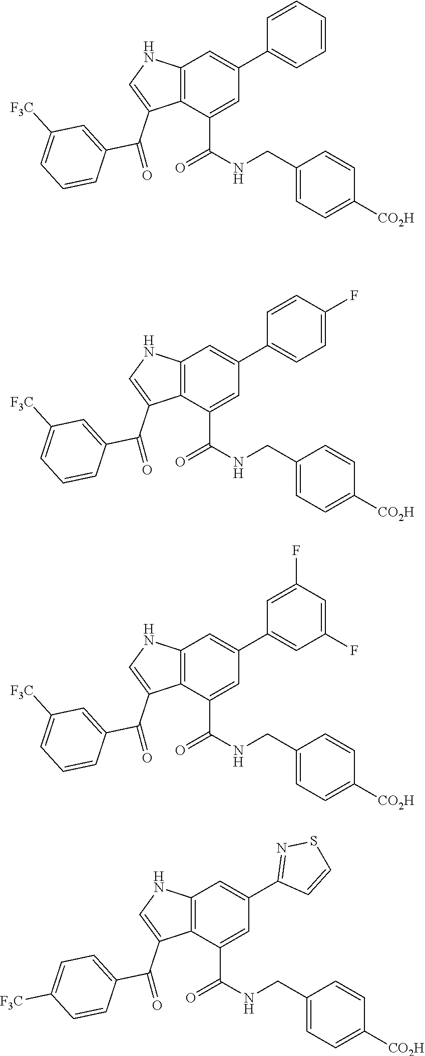

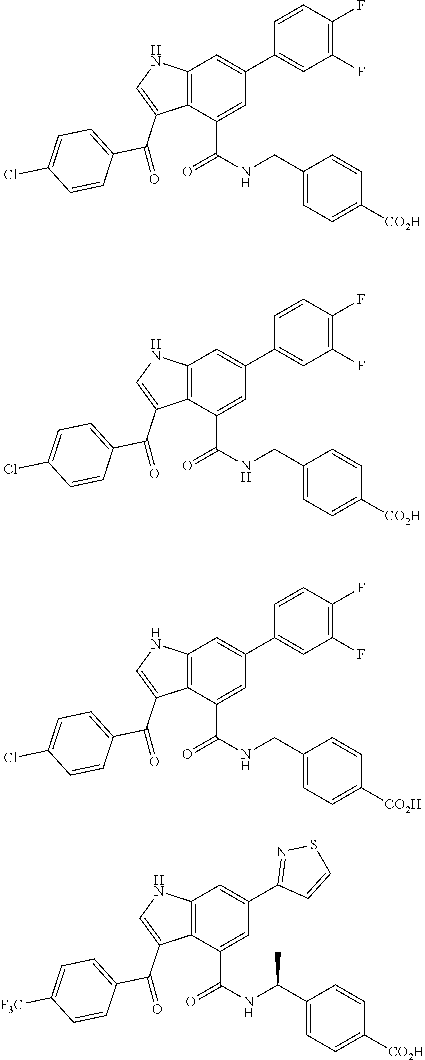

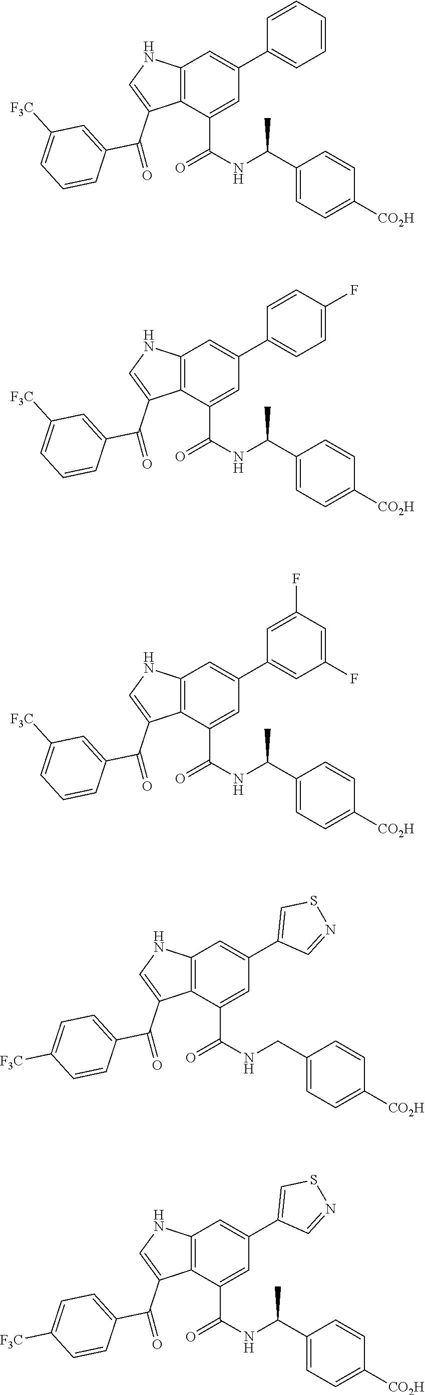 [SMILES: C[C@H](NC(=O)c1cc(-c2ccccc2)cc2[nH]cc(C(=O)c3cccc(C(F)(F)F)c3)c12)c1ccc(C(=O)O)cc1 ; C[C@H](NC(=O)c1cc(-c2ccc(F)cc2)cc2[nH]cc(C(=O)c3cccc(C(F)(F)F)c3)c12)c1ccc(C(=O)O)cc1 ; C[C@H](NC(=O)c1cc(-c2cc(F)cc(F)c2)cc2[nH]cc(C(=O)c3cccc(C(F)(F)F)c3)c12)c1ccc(C(=O)O)cc1 ; O=C(O)c1ccc(CNC(=O)c2cc(-c3cnsc3)cc3[nH]cc(C(=O)c4ccc(C(F)(F)F)cc4)c23)cc1 ; C[C@H](NC(=O)c1cc(-c2cnsc2)cc2[nH]cc(C(=O)c3ccc(C(F)(F)F)cc3)c12)c1ccc(C(=O)O)cc1]

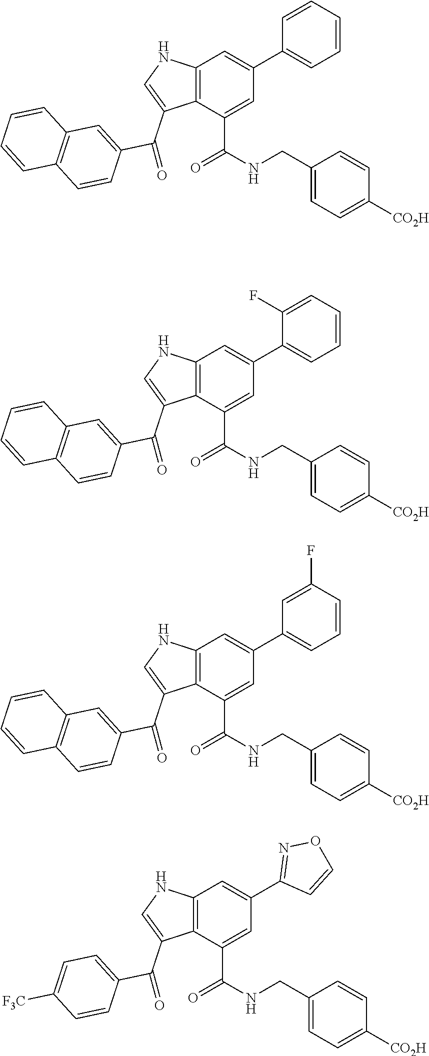 [SMILES: O=C(O)c1ccc(CNC(=O)c2cc(-c3ccccc3)cc3[nH]cc(C(=O)c4ccc5ccccc5c4)c23)cc1 ; O=C(O)c1ccc(CNC(=O)c2cc(-c3ccccc3F)cc3[nH]cc(C(=O)c4ccc5ccccc5c4)c23)cc1 ; O=C(O)c1ccc(CNC(=O)c2cc(-c3cccc(F)c3)cc3[nH]cc(C(=O)c4ccc5ccccc5c4)c23)cc1 ; O=C(O)c1ccc(CNC(=O)c2cc(-c3ccon3)cc3[nH]cc(C(=O)c4ccc(C(F)(F)F)cc4)c23)cc1]

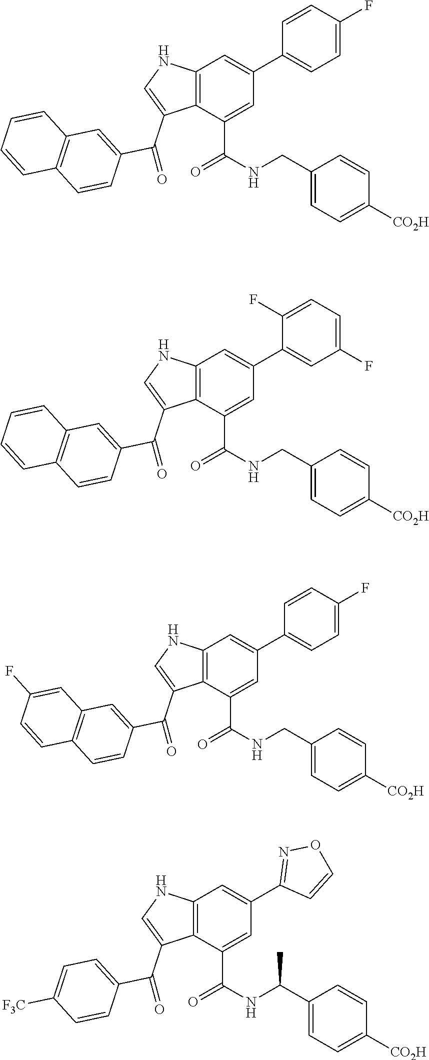 [SMILES: O=C(O)c1ccc(CNC(=O)c2cc(-c3ccc(F)cc3)cc3[nH]cc(C(=O)c4ccc5ccccc5c4)c23)cc1 ; O=C(O)c1ccc(CNC(=O)c2cc(-c3cc(F)ccc3F)cc3[nH]cc(C(=O)c4ccc5ccccc5c4)c23)cc1 ; O=C(O)c1ccc(CNC(=O)c2cc(-c3ccc(F)cc3)cc3[nH]cc(C(=O)c4ccc5ccc(F)cc5c4)c23)cc1 ; C[C@H](NC(=O)c1cc(-c2ccon2)cc2[nH]cc(C(=O)c3ccc(C(F)(F)F)cc3)c12)c1ccc(C(=O)O)cc1]

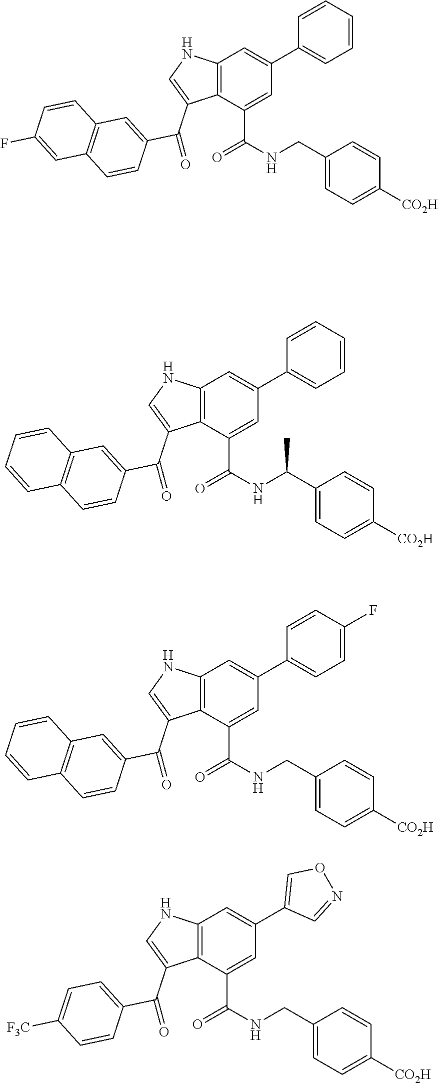 [SMILES: O=C(O)c1ccc(CNC(=O)c2cc(-c3ccccc3)cc3[nH]cc(C(=O)c4ccc5cc(F)ccc5c4)c23)cc1 ; C[C@H](NC(=O)c1cc(-c2ccccc2)cc2[nH]cc(C(=O)c3ccc4ccccc4c3)c12)c1ccc(C(=O)O)cc1 ; O=C(O)c1ccc(CNC(=O)c2cc(-c3ccc(F)cc3)cc3[nH]cc(C(=O)c4ccc5ccccc5c4)c23)cc1 ; O=C(O)c1ccc(CNC(=O)c2cc(-c3cnoc3)cc3[nH]cc(C(=O)c4ccc(C(F)(F)F)cc4)c23)cc1]

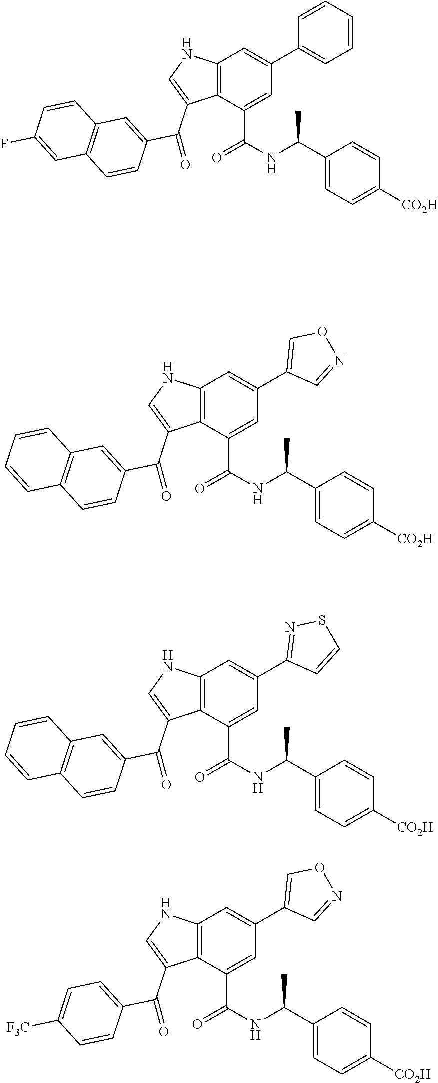 [SMILES: C[C@H](NC(=O)c1cc(-c2ccccc2)cc2[nH]cc(C(=O)c3ccc4cc(F)ccc4c3)c12)c1ccc(C(=O)O)cc1 ; C[C@H](NC(=O)c1cc(-c2cnoc2)cc2[nH]cc(C(=O)c3ccc4ccccc4c3)c12)c1ccc(C(=O)O)cc1 ; C[C@H](NC(=O)c1cc(-c2ccsn2)cc2[nH]cc(C(=O)c3ccc4ccccc4c3)c12)c1ccc(C(=O)O)cc1 ; C[C@H](NC(=O)c1cc(-c2cnoc2)cc2[nH]cc(C(=O)c3ccc(C(F)(F)F)cc3)c12)c1ccc(C(=O)O)cc1]

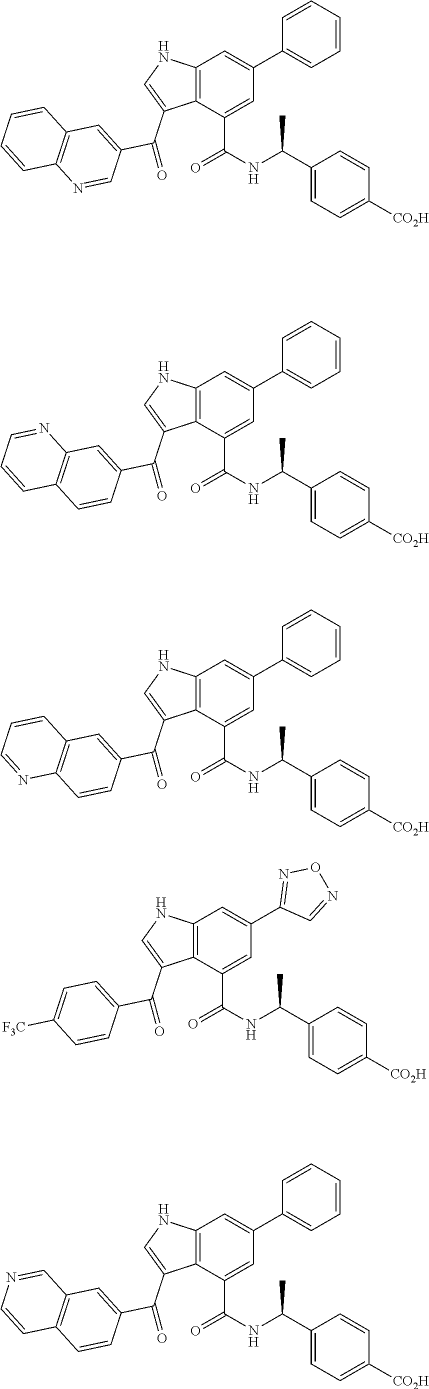 [SMILES: C[C@H](NC(=O)c1cc(-c2ccccc2)cc2[nH]cc(C(=O)c3cnc4ccccc4c3)c12)c1ccc(C(=O)O)cc1 ; C[C@H](NC(=O)c1cc(-c2ccccc2)cc2[nH]cc(C(=O)c3ccc4cccnc4c3)c12)c1ccc(C(=O)O)cc1 ; C[C@H](NC(=O)c1cc(-c2ccccc2)cc2[nH]cc(C(=O)c3ccc4ncccc4c3)c12)c1ccc(C(=O)O)cc1 ; C[C@H](NC(=O)c1cc(-c2cnon2)cc2[nH]cc(C(=O)c3ccc(C(F)(F)F)cc3)c12)c1ccc(C(=O)O)cc1 ; C[C@H](NC(=O)c1cc(-c2ccccc2)cc2[nH]cc(C(=O)c3ccc4ccncc4c3)c12)c1ccc(C(=O)O)cc1]

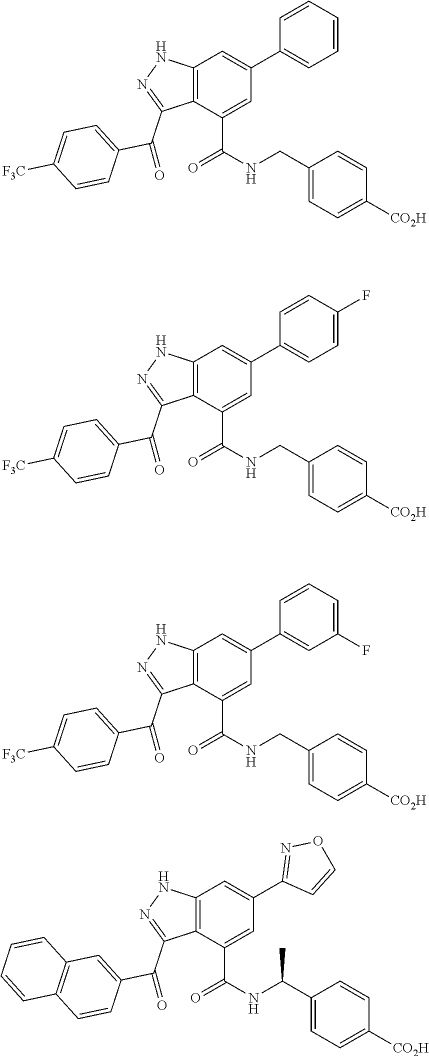

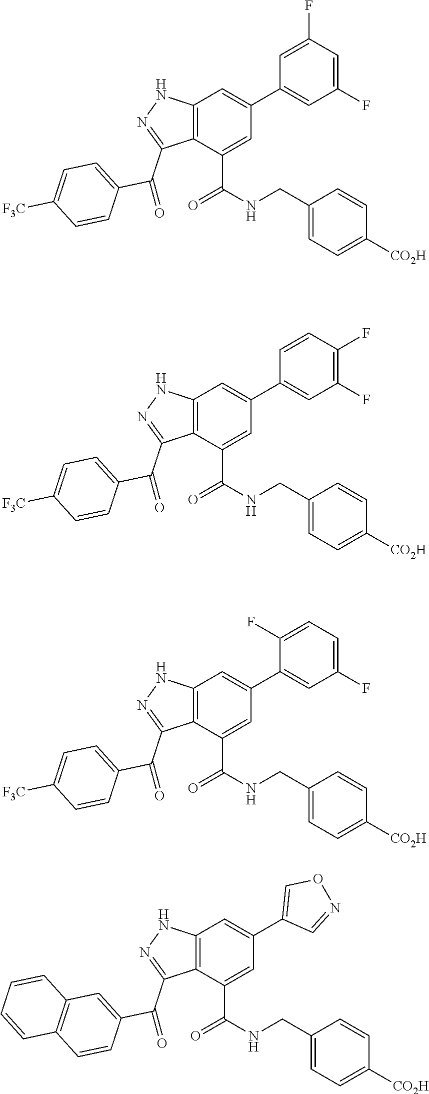

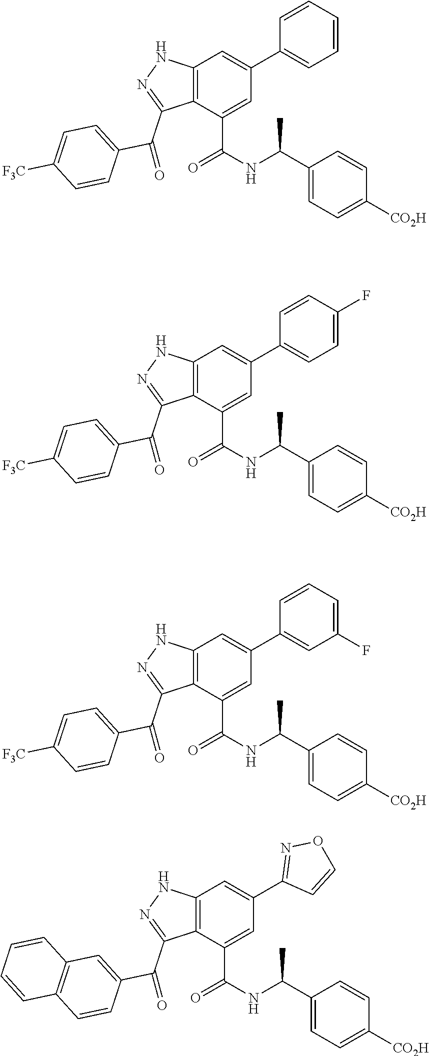

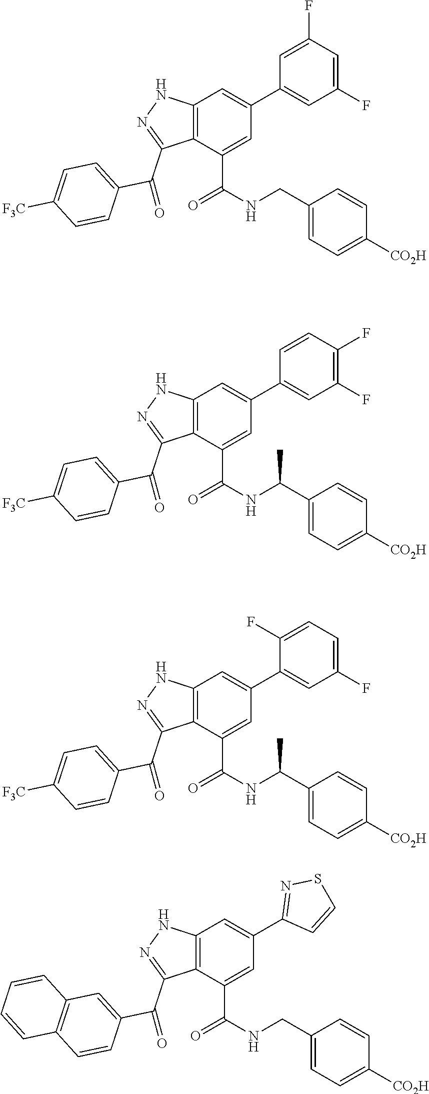

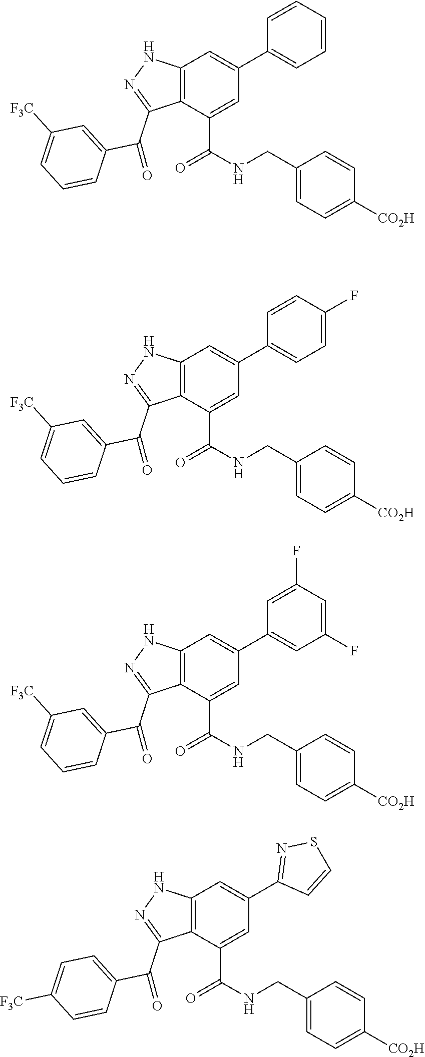

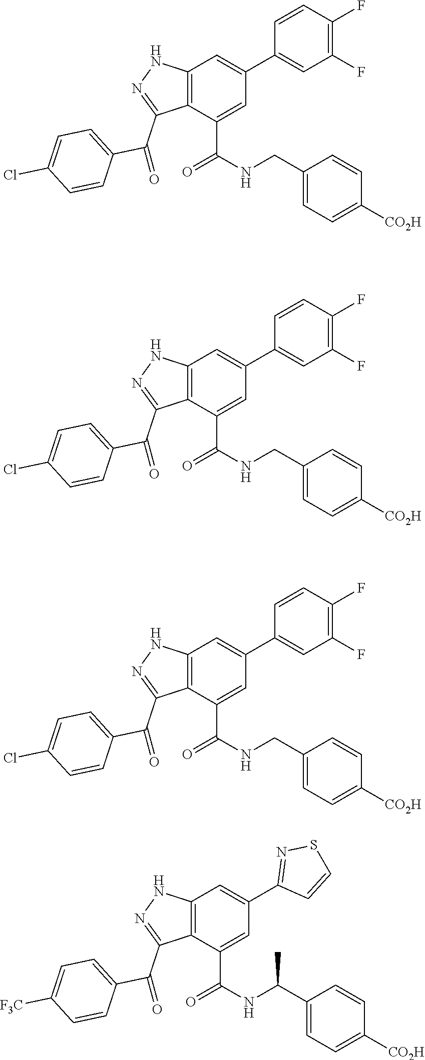

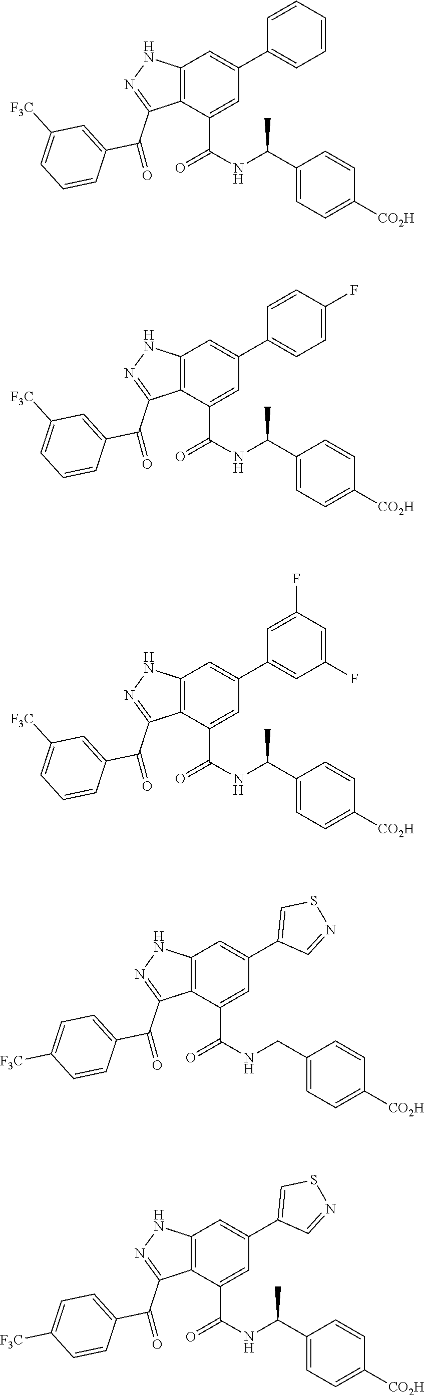 [SMILES: C[C@H](NC(=O)c1cc(-c2ccccc2)cc2[nH]nc(C(=O)c3cccc(C(F)(F)F)c3)c12)c1ccc(C(=O)O)cc1 ; C[C@H](NC(=O)c1cc(-c2ccc(F)cc2)cc2[nH]nc(C(=O)c3cccc(C(F)(F)F)c3)c12)c1ccc(C(=O)O)cc1 ; C[C@H](NC(=O)c1cc(-c2cc(F)cc(F)c2)cc2[nH]nc(C(=O)c3cccc(C(F)(F)F)c3)c12)c1ccc(C(=O)O)cc1 ; O=C(O)c1ccc(CNC(=O)c2cc(-c3cnsc3)cc3[nH]nc(C(=O)c4ccc(C(F)(F)F)cc4)c23)cc1 ; C[C@H](NC(=O)c1cc(-c2cnsc2)cc2[nH]nc(C(=O)c3ccc(C(F)(F)F)cc3)c12)c1ccc(C(=O)O)cc1]

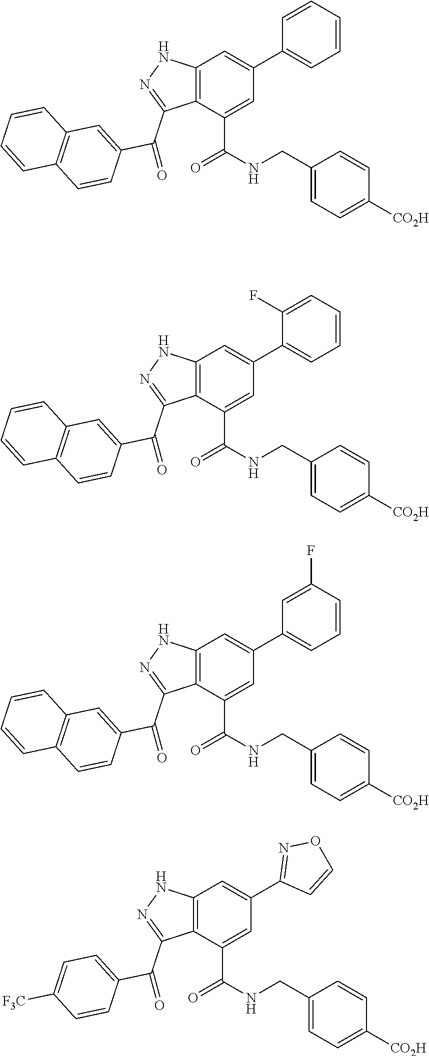 [SMILES: O=C(O)c1ccc(CNC(=O)c2cc(-c3ccccc3)cc3[nH]nc(C(=O)c4ccc5ccccc5c4)c23)cc1 ; O=C(O)c1ccc(CNC(=O)c2cc(-c3ccccc3F)cc3[nH]nc(C(=O)c4ccc5ccccc5c4)c23)cc1 ; O=C(O)c1ccc(CNC(=O)c2cc(-c3cccc(F)c3)cc3[nH]nc(C(=O)c4ccc5ccccc5c4)c23)cc1 ; O=C(O)c1ccc(CNC(=O)c2cc(-c3ccon3)cc3[nH]nc(C(=O)c4ccc(C(F)(F)F)cc4)c23)cc1]

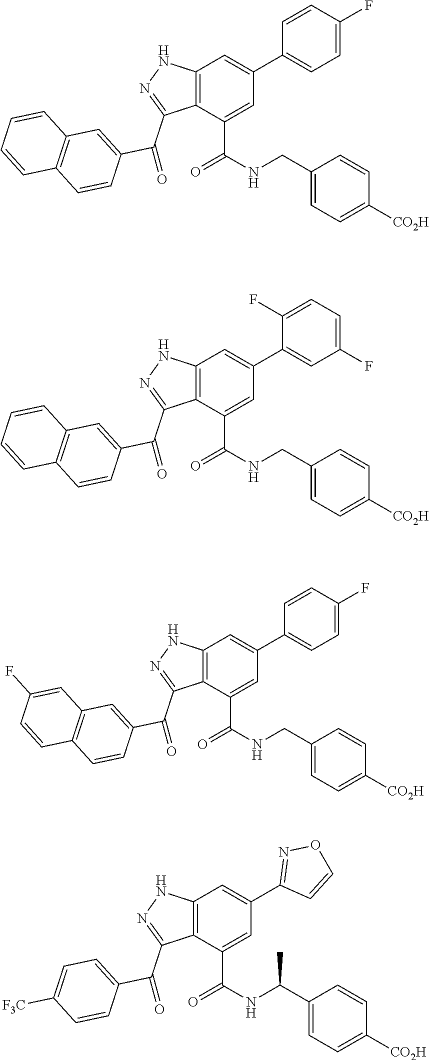 [SMILES: O=C(O)c1ccc(CNC(=O)c2cc(-c3ccc(F)cc3)cc3[nH]nc(C(=O)c4ccc5ccccc5c4)c23)cc1 ; O=C(O)c1ccc(CNC(=O)c2cc(-c3cc(F)ccc3F)cc3[nH]nc(C(=O)c4ccc5ccccc5c4)c23)cc1 ; O=C(O)c1ccc(CNC(=O)c2cc(-c3ccc(F)cc3)cc3[nH]nc(C(=O)c4ccc5ccc(F)cc5c4)c23)cc1 ; C[C@H](NC(=O)c1cc(-c2ccon2)cc2[nH]nc(C(=O)c3ccc(C(F)(F)F)cc3)c12)c1ccc(C(=O)O)cc1]

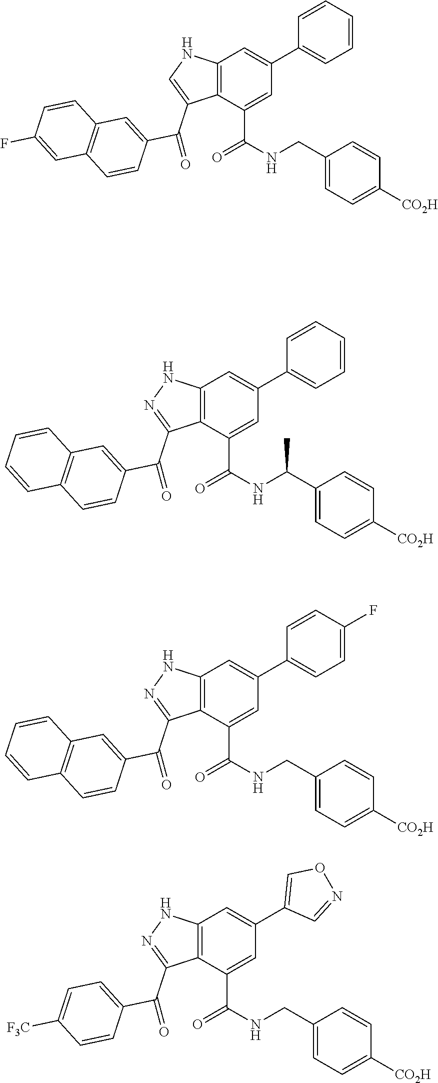 [SMILES: O=C(O)c1ccc(CNC(=O)c2cc(-c3ccccc3)cc3[nH]cc(C(=O)c4ccc5cc(F)ccc5c4)c23)cc1 ; C[C@H](NC(=O)c1cc(-c2ccccc2)cc2[nH]nc(C(=O)c3ccc4ccccc4c3)c12)c1ccc(C(=O)O)cc1 ; O=C(O)c1ccc(CNC(=O)c2cc(-c3ccc(F)cc3)cc3[nH]nc(C(=O)c4ccc5ccccc5c4)c23)cc1 ; O=C(O)c1ccc(CNC(=O)c2cc(-c3cnoc3)cc3[nH]nc(C(=O)c4ccc(C(F)(F)F)cc4)c23)cc1]

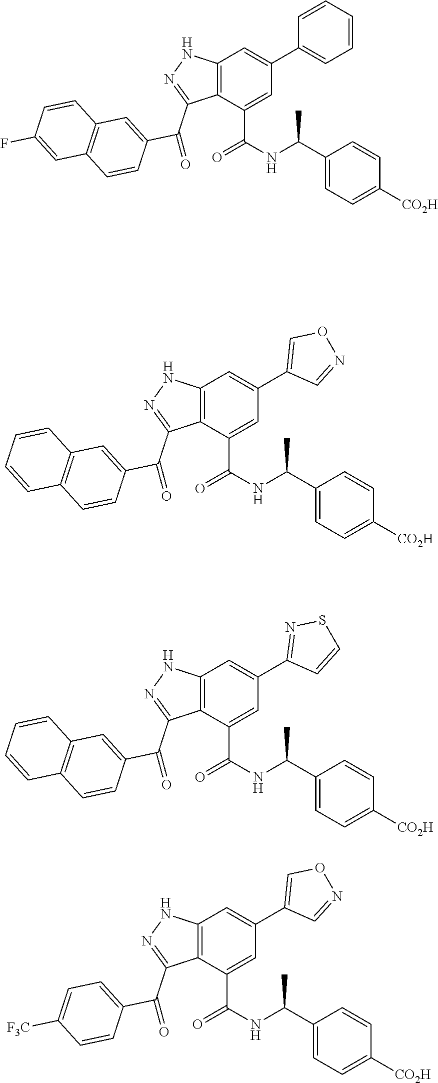 [SMILES: C[C@H](NC(=O)c1cc(-c2ccccc2)cc2[nH]nc(C(=O)c3ccc4cc(F)ccc4c3)c12)c1ccc(C(=O)O)cc1 ; C[C@H](NC(=O)c1cc(-c2cnoc2)cc2[nH]nc(C(=O)c3ccc4ccccc4c3)c12)c1ccc(C(=O)O)cc1 ; C[C@H](NC(=O)c1cc(-c2ccsn2)cc2[nH]nc(C(=O)c3ccc4ccccc4c3)c12)c1ccc(C(=O)O)cc1 ; C[C@H](NC(=O)c1cc(-c2cnoc2)cc2[nH]nc(C(=O)c3ccc(C(F)(F)F)cc3)c12)c1ccc(C(=O)O)cc1]

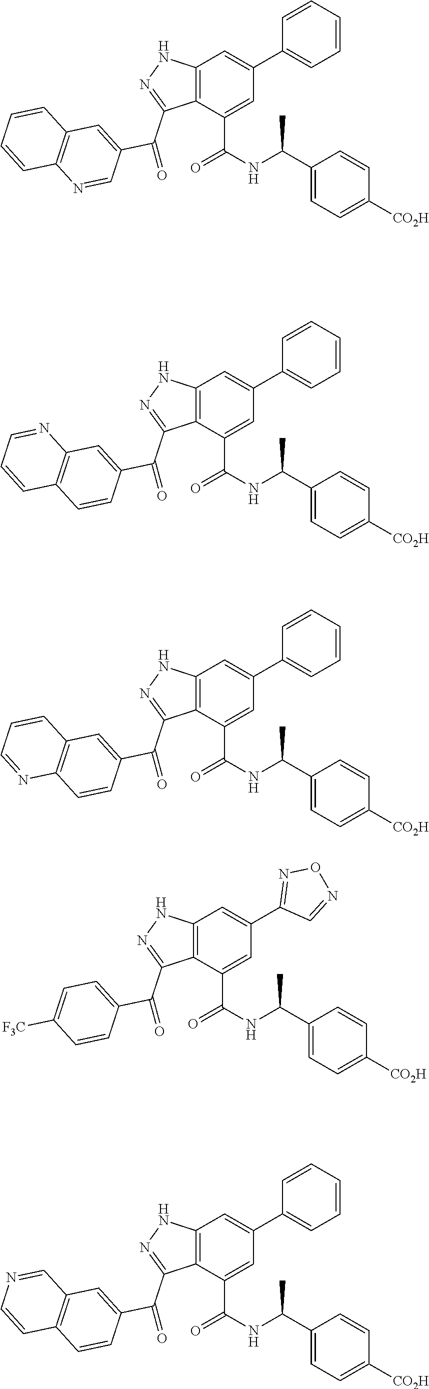 [SMILES: C[C@H](NC(=O)c1cc(-c2ccccc2)cc2[nH]nc(C(=O)c3cnc4ccccc4c3)c12)c1ccc(C(=O)O)cc1 ; C[C@H](NC(=O)c1cc(-c2ccccc2)cc2[nH]nc(C(=O)c3ccc4cccnc4c3)c12)c1ccc(C(=O)O)cc1 ; C[C@H](NC(=O)c1cc(-c2ccccc2)cc2[nH]nc(C(=O)c3ccc4ncccc4c3)c12)c1ccc(C(=O)O)cc1 ; C[C@H](NC(=O)c1cc(-c2cnon2)cc2[nH]nc(C(=O)c3ccc(C(F)(F)F)cc3)c12)c1ccc(C(=O)O)cc1 ; C[C@H](NC(=O)c1cc(-c2ccccc2)cc2[nH]nc(C(=O)c3ccc4ccncc4c3)c12)c1ccc(C(=O)O)cc1]

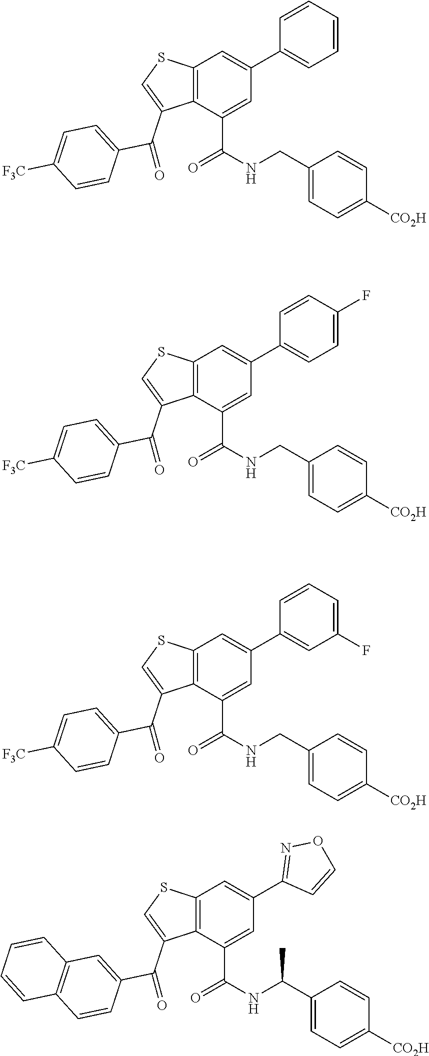

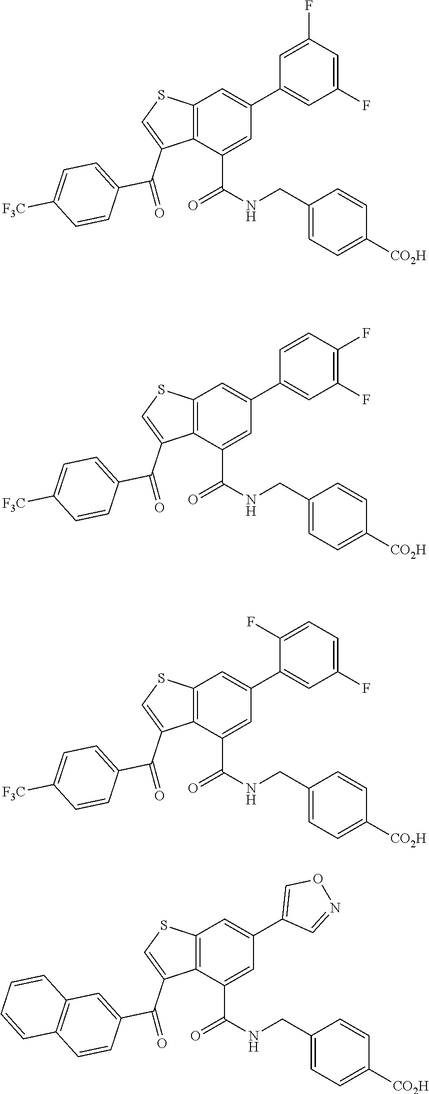

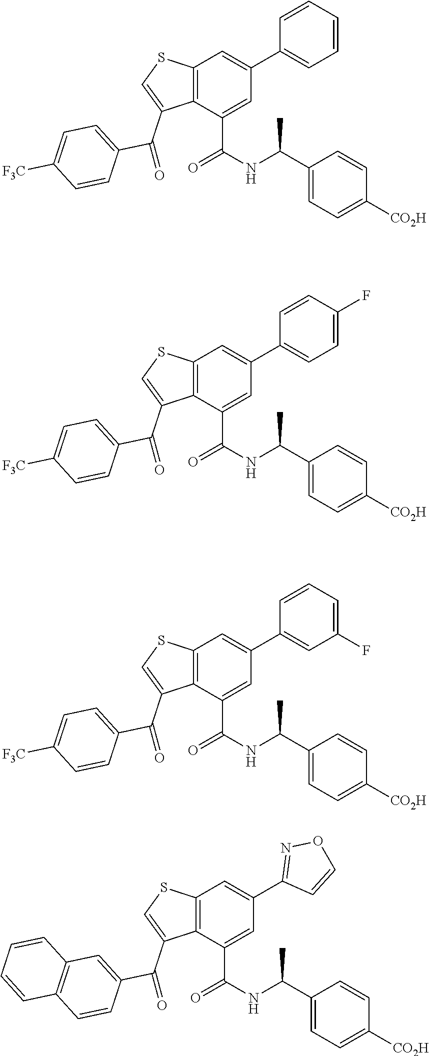

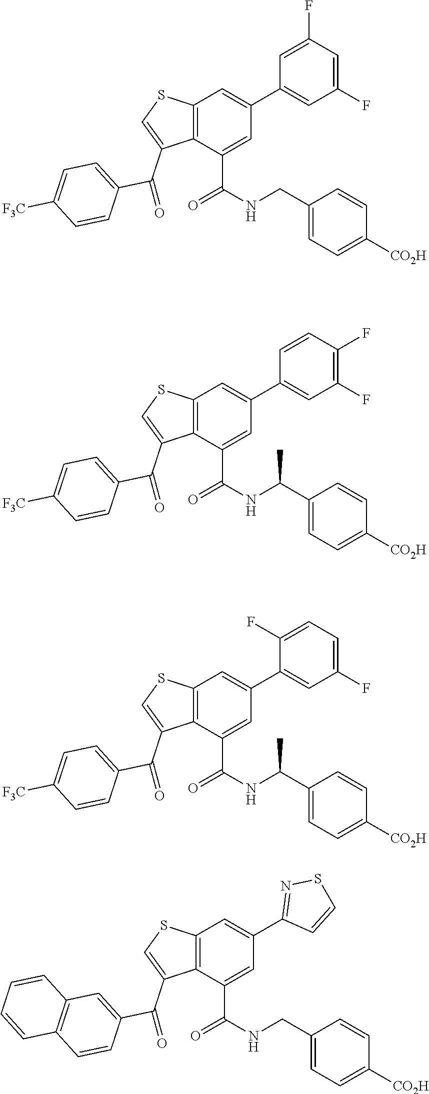

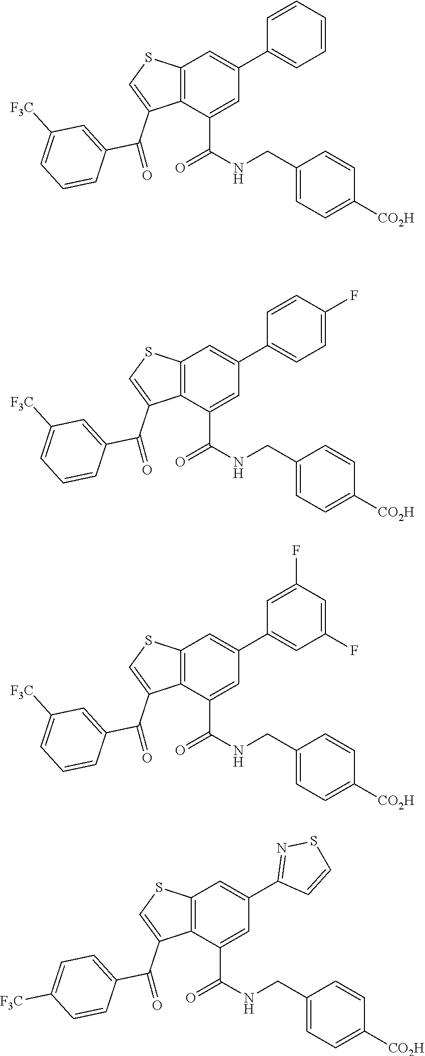

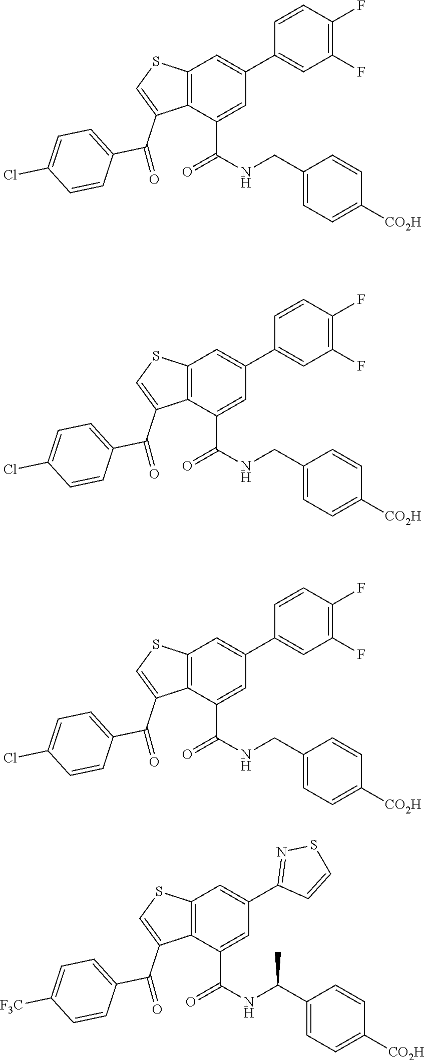

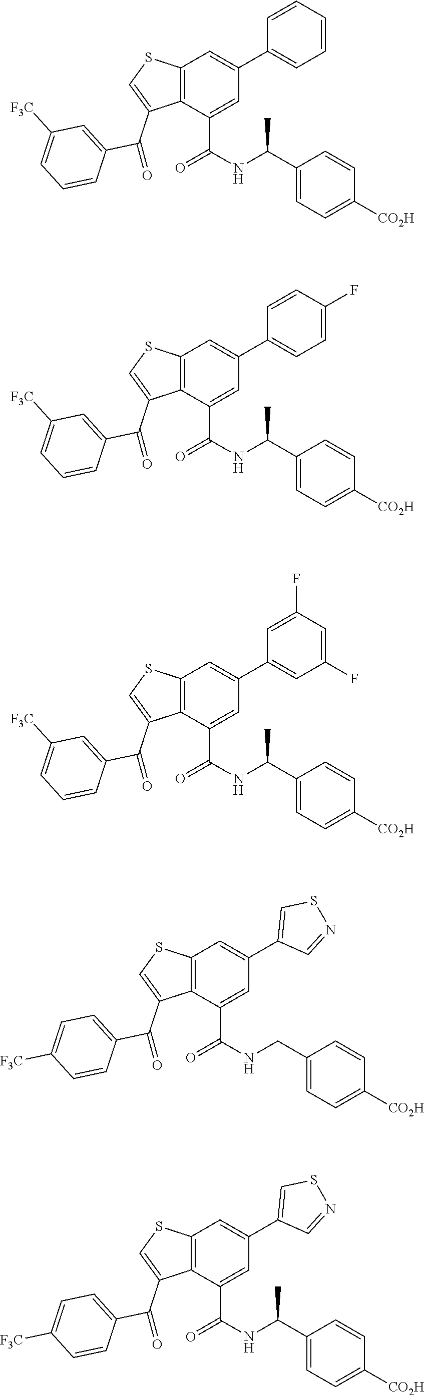 [SMILES: C[C@H](NC(=O)c1cc(-c2ccccc2)cc2scc(C(=O)c3cccc(C(F)(F)F)c3)c12)c1ccc(C(=O)O)cc1 ; C[C@H](NC(=O)c1cc(-c2ccc(F)cc2)cc2scc(C(=O)c3cccc(C(F)(F)F)c3)c12)c1ccc(C(=O)O)cc1 ; C[C@H](NC(=O)c1cc(-c2cc(F)cc(F)c2)cc2scc(C(=O)c3cccc(C(F)(F)F)c3)c12)c1ccc(C(=O)O)cc1 ; O=C(O)c1ccc(CNC(=O)c2cc(-c3cnsc3)cc3scc(C(=O)c4ccc(C(F)(F)F)cc4)c23)cc1 ; C[C@H](NC(=O)c1cc(-c2cnsc2)cc2scc(C(=O)c3ccc(C(F)(F)F)cc3)c12)c1ccc(C(=O)O)cc1]

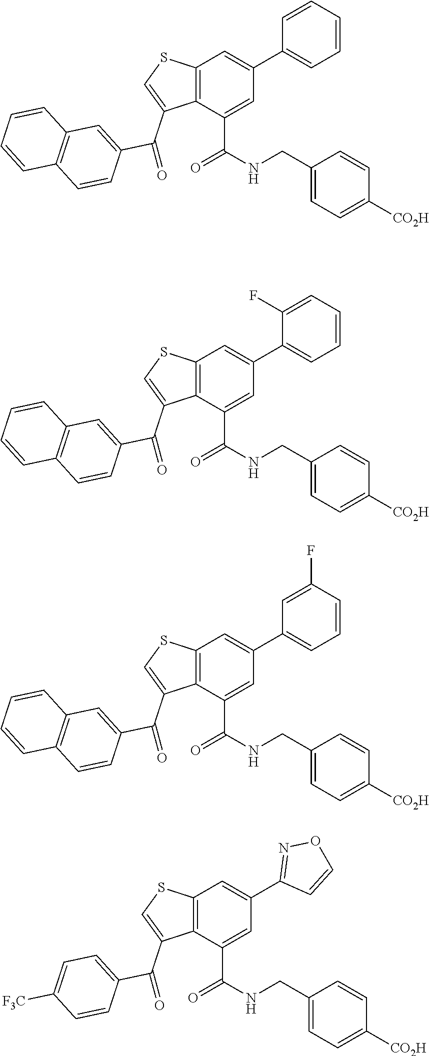 [SMILES: O=C(O)c1ccc(CNC(=O)c2cc(-c3ccccc3)cc3scc(C(=O)c4ccc5ccccc5c4)c23)cc1 ; O=C(O)c1ccc(CNC(=O)c2cc(-c3ccccc3F)cc3scc(C(=O)c4ccc5ccccc5c4)c23)cc1 ; O=C(O)c1ccc(CNC(=O)c2cc(-c3cccc(F)c3)cc3scc(C(=O)c4ccc5ccccc5c4)c23)cc1 ; O=C(O)c1ccc(CNC(=O)c2cc(-c3ccon3)cc3scc(C(=O)c4ccc(C(F)(F)F)cc4)c23)cc1]

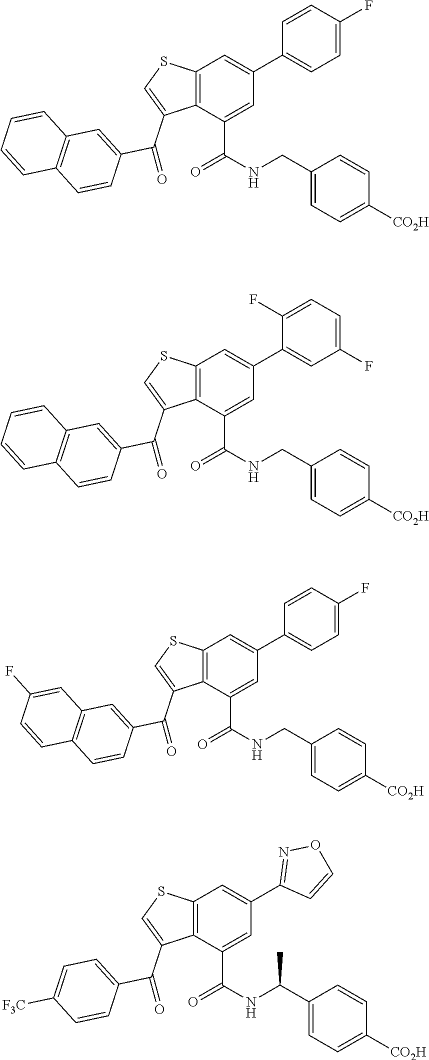 [SMILES: O=C(O)c1ccc(CNC(=O)c2cc(-c3ccc(F)cc3)cc3scc(C(=O)c4ccc5ccccc5c4)c23)cc1 ; O=C(O)c1ccc(CNC(=O)c2cc(-c3cc(F)ccc3F)cc3scc(C(=O)c4ccc5ccccc5c4)c23)cc1 ; O=C(O)c1ccc(CNC(=O)c2cc(-c3ccc(F)cc3)cc3scc(C(=O)c4ccc5ccc(F)cc5c4)c23)cc1 ; C[C@H](NC(=O)c1cc(-c2ccon2)cc2scc(C(=O)c3ccc(C(F)(F)F)cc3)c12)c1ccc(C(=O)O)cc1]

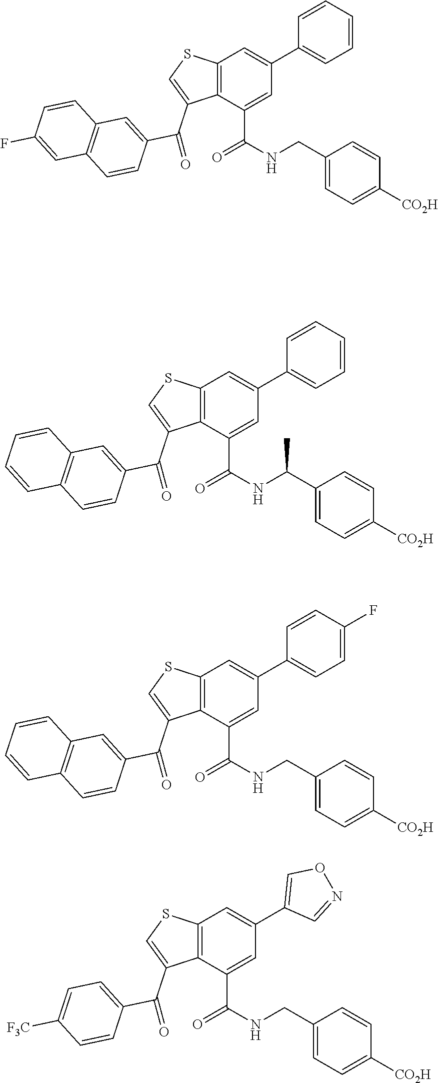 [SMILES: O=C(O)c1ccc(CNC(=O)c2cc(-c3ccccc3)cc3scc(C(=O)c4ccc5cc(F)ccc5c4)c23)cc1 ; C[C@H](NC(=O)c1cc(-c2ccccc2)cc2scc(C(=O)c3ccc4ccccc4c3)c12)c1ccc(C(=O)O)cc1 ; O=C(O)c1ccc(CNC(=O)c2cc(-c3ccc(F)cc3)cc3scc(C(=O)c4ccc5ccccc5c4)c23)cc1 ; O=C(O)c1ccc(CNC(=O)c2cc(-c3cnoc3)cc3scc(C(=O)c4ccc(C(F)(F)F)cc4)c23)cc1]

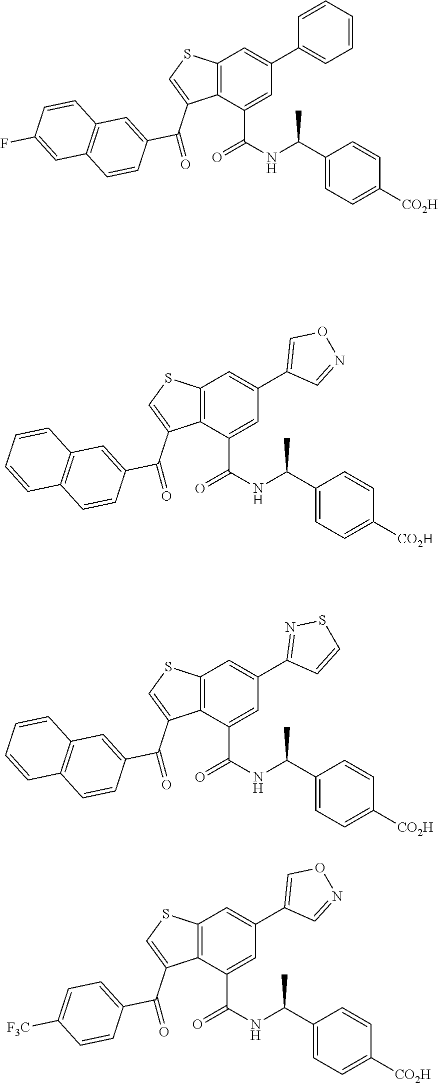 [SMILES: C[C@H](NC(=O)c1cc(-c2ccccc2)cc2scc(C(=O)c3ccc4cc(F)ccc4c3)c12)c1ccc(C(=O)O)cc1 ; C[C@H](NC(=O)c1cc(-c2cnoc2)cc2scc(C(=O)c3ccc4ccccc4c3)c12)c1ccc(C(=O)O)cc1 ; C[C@H](NC(=O)c1cc(-c2ccsn2)cc2scc(C(=O)c3ccc4ccccc4c3)c12)c1ccc(C(=O)O)cc1 ; C[C@H](NC(=O)c1cc(-c2cnoc2)cc2scc(C(=O)c3ccc(C(F)(F)F)cc3)c12)c1ccc(C(=O)O)cc1]

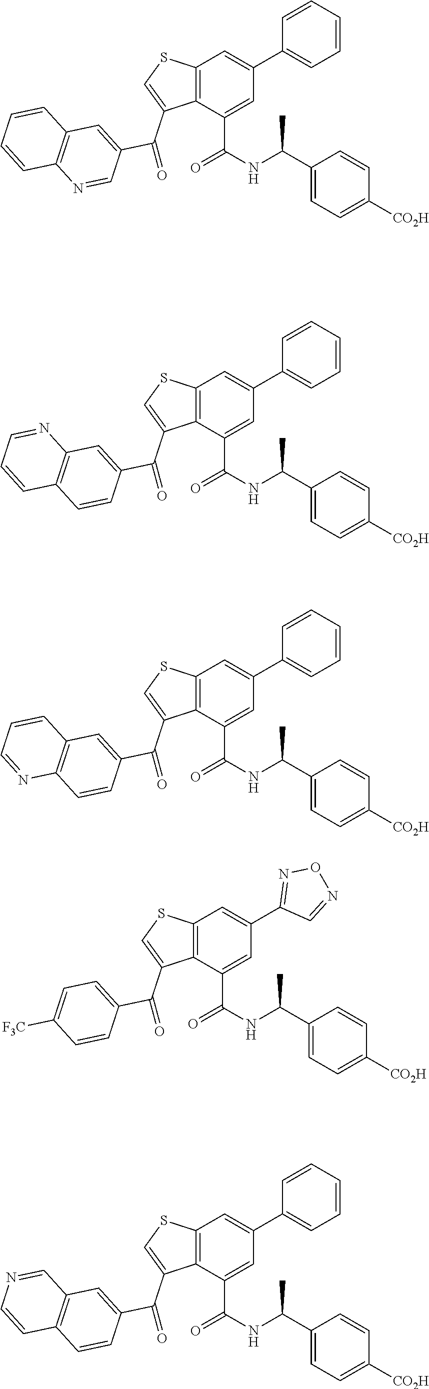 [SMILES: C[C@H](NC(=O)c1cc(-c2ccccc2)cc2scc(C(=O)c3cnc4ccccc4c3)c12)c1ccc(C(=O)O)cc1 ; C[C@H](NC(=O)c1cc(-c2ccccc2)cc2scc(C(=O)c3ccc4cccnc4c3)c12)c1ccc(C(=O)O)cc1 ; C[C@H](NC(=O)c1cc(-c2ccccc2)cc2scc(C(=O)c3ccc4ncccc4c3)c12)c1ccc(C(=O)O)cc1 ; C[C@H](NC(=O)c1cc(-c2cnon2)cc2scc(C(=O)c3ccc(C(F)(F)F)cc3)c12)c1ccc(C(=O)O)cc1 ; C[C@H](NC(=O)c1cc(-c2ccccc2)cc2scc(C(=O)c3ccc4ccncc4c3)c12)c1ccc(C(=O)O)cc1]

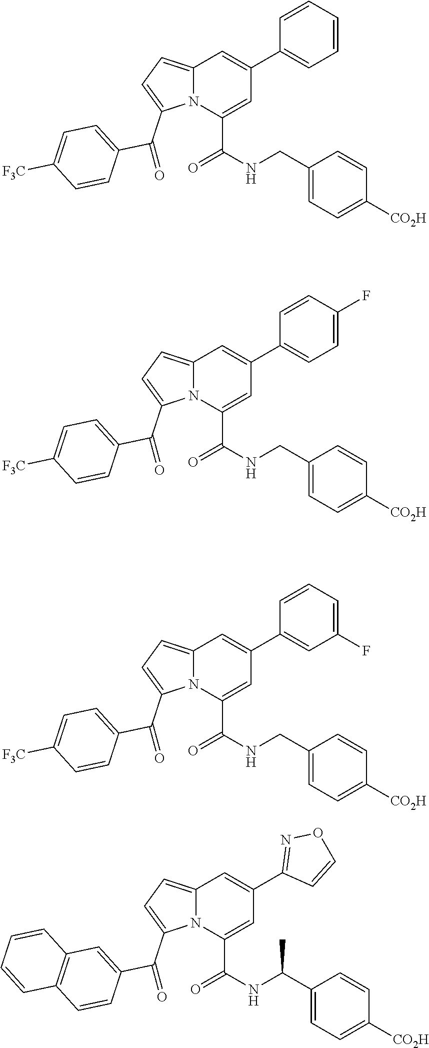

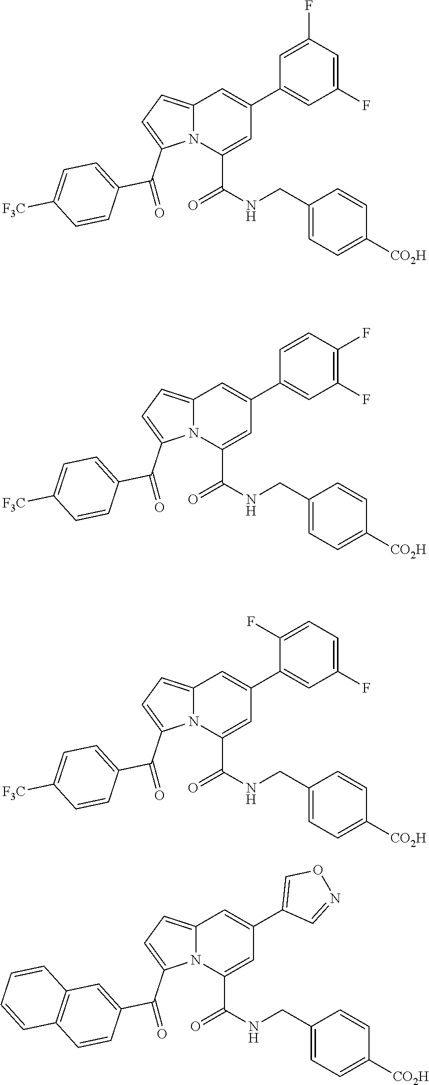

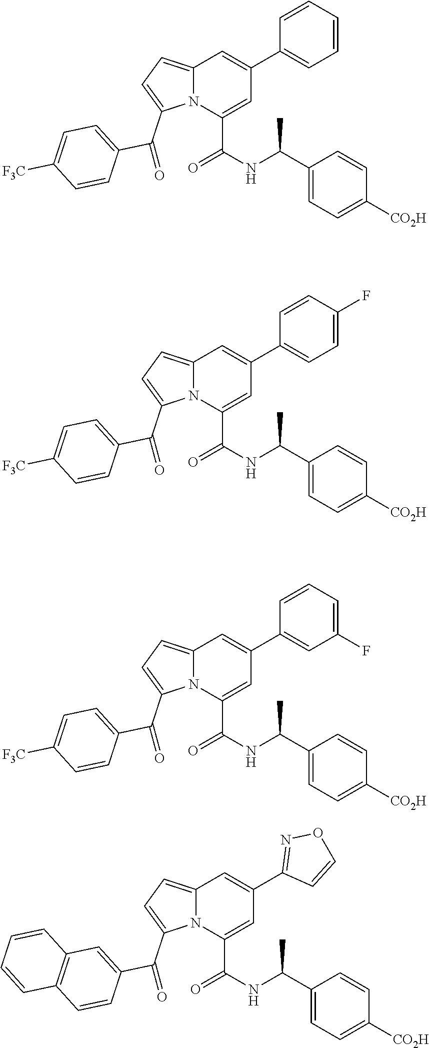

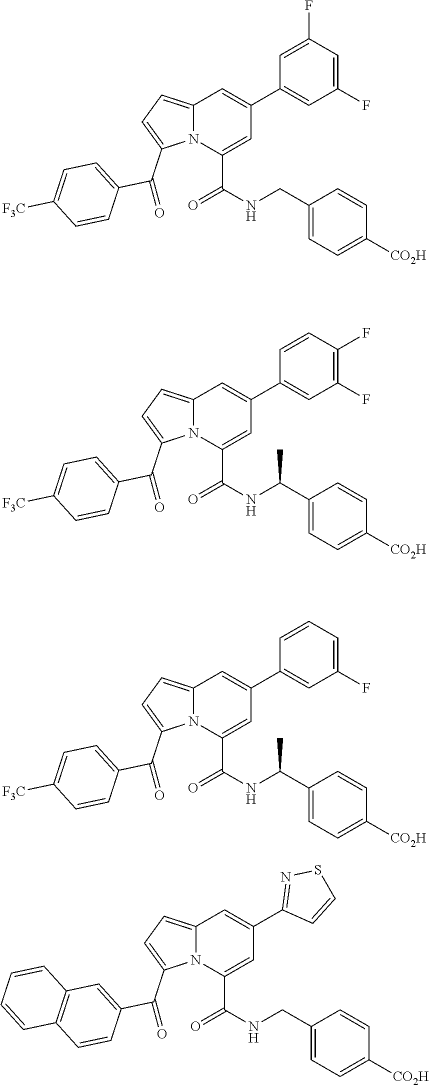

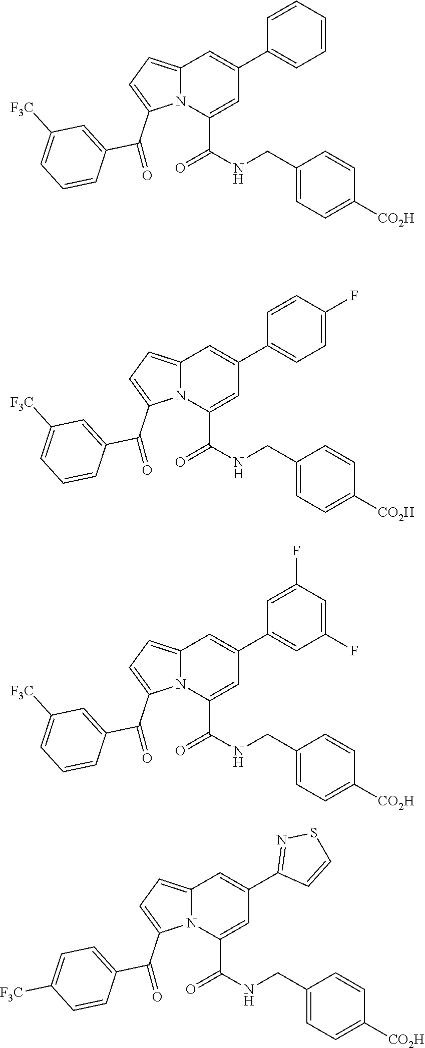

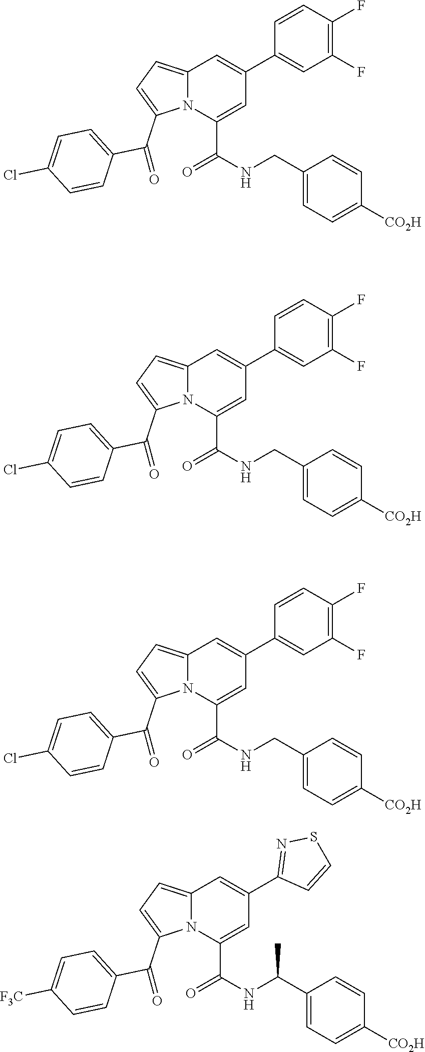

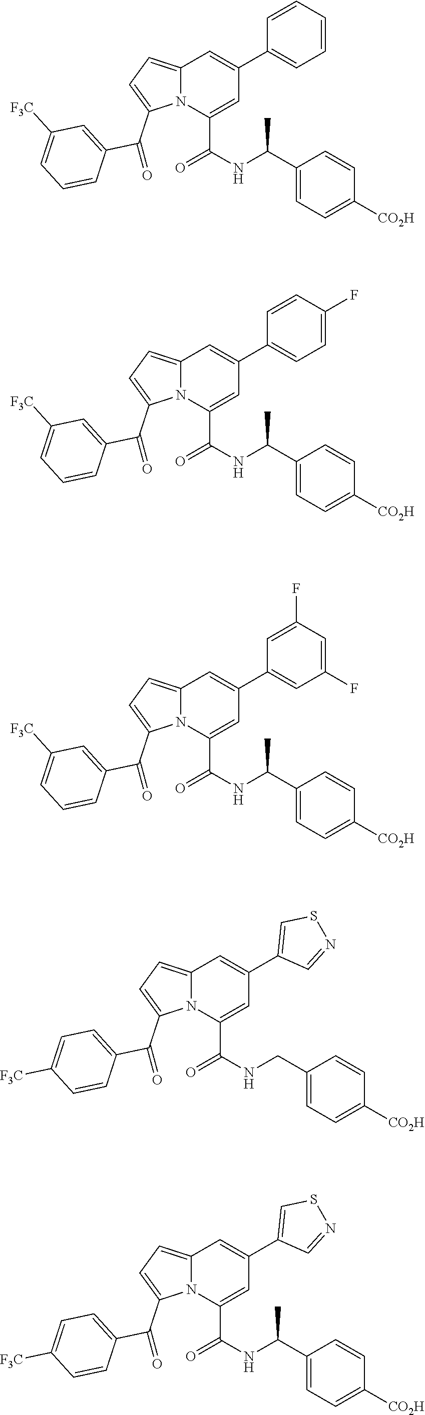 [SMILES: C[C@H](NC(=O)c1cc(-c2ccccc2)cc2ccc(C(=O)c3cccc(C(F)(F)F)c3)n12)c1ccc(C(=O)O)cc1 ; C[C@H](NC(=O)c1cc(-c2ccc(F)cc2)cc2ccc(C(=O)c3cccc(C(F)(F)F)c3)n12)c1ccc(C(=O)O)cc1 ; C[C@H](NC(=O)c1cc(-c2cc(F)cc(F)c2)cc2ccc(C(=O)c3cccc(C(F)(F)F)c3)n12)c1ccc(C(=O)O)cc1 ; O=C(O)c1ccc(CNC(=O)c2cc(-c3cnsc3)cc3ccc(C(=O)c4ccc(C(F)(F)F)cc4)n23)cc1 ; C[C@H](NC(=O)c1cc(-c2cnsc2)cc2ccc(C(=O)c3ccc(C(F)(F)F)cc3)n12)c1ccc(C(=O)O)cc1]

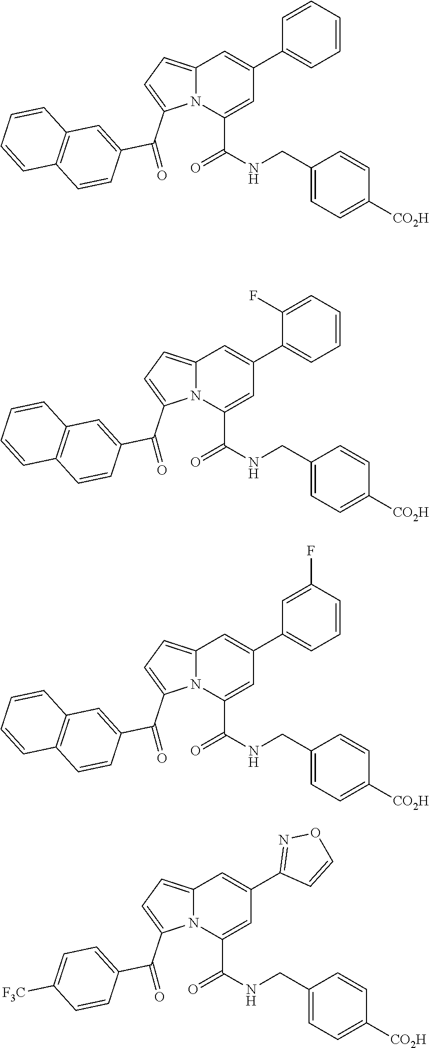

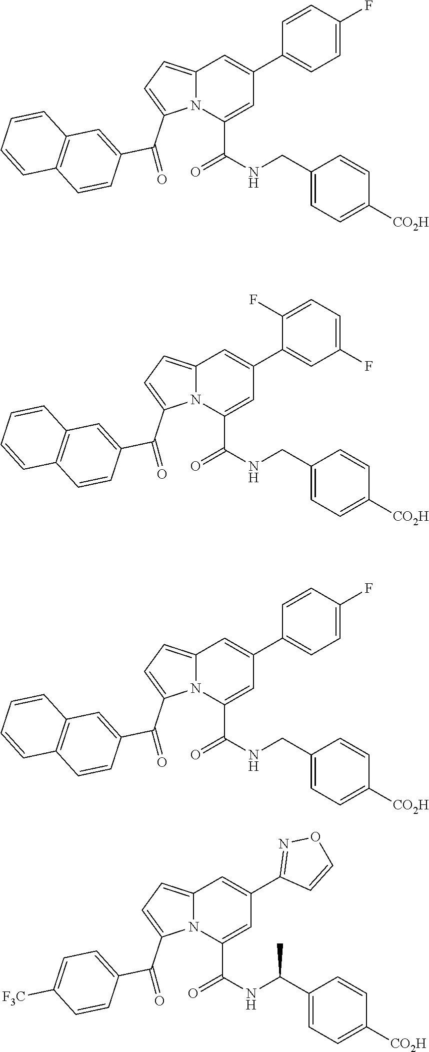

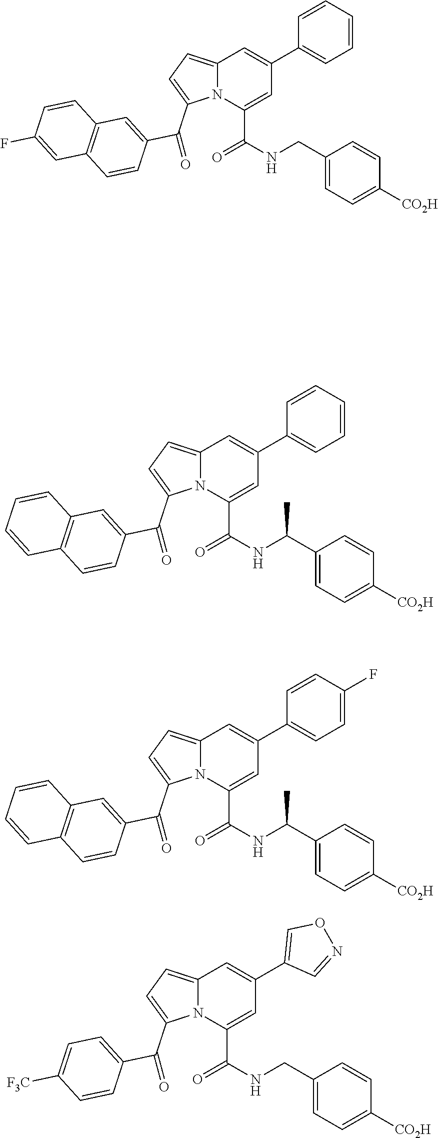

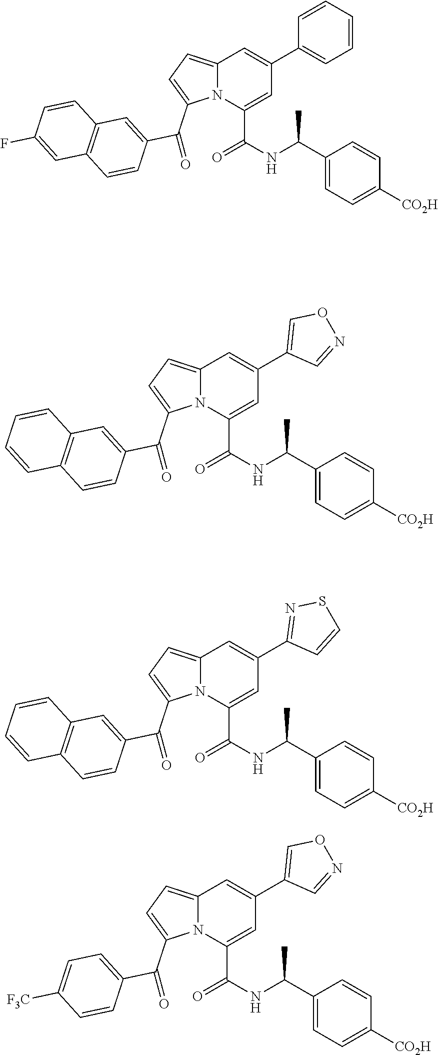

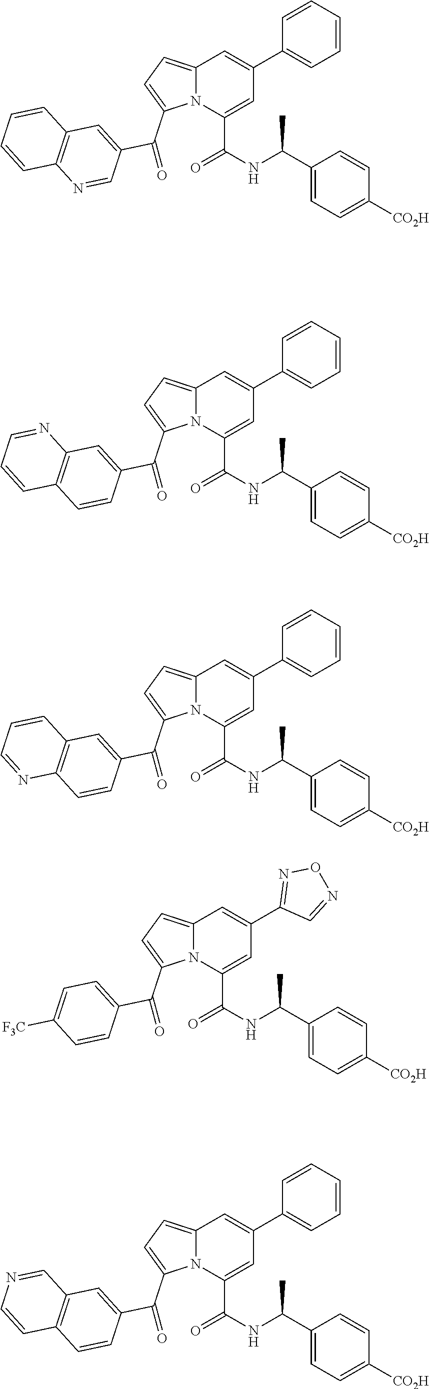 [SMILES: C[C@H](NC(=O)c1cc(-c2ccccc2)cc2ccc(C(=O)c3cnc4ccccc4c3)n12)c1ccc(C(=O)O)cc1 ; C[C@H](NC(=O)c1cc(-c2ccccc2)cc2ccc(C(=O)c3ccc4cccnc4c3)n12)c1ccc(C(=O)O)cc1 ; C[C@H](NC(=O)c1cc(-c2ccccc2)cc2ccc(C(=O)c3ccc4ncccc4c3)n12)c1ccc(C(=O)O)cc1 ; C[C@H](NC(=O)c1cc(-c2cnon2)cc2ccc(C(=O)c3ccc(C(F)(F)F)cc3)n12)c1ccc(C(=O)O)cc1 ; C[C@H](NC(=O)c1cc(-c2ccccc2)cc2ccc(C(=O)c3ccc4ccncc4c3)n12)c1ccc(C(=O)O)cc1]

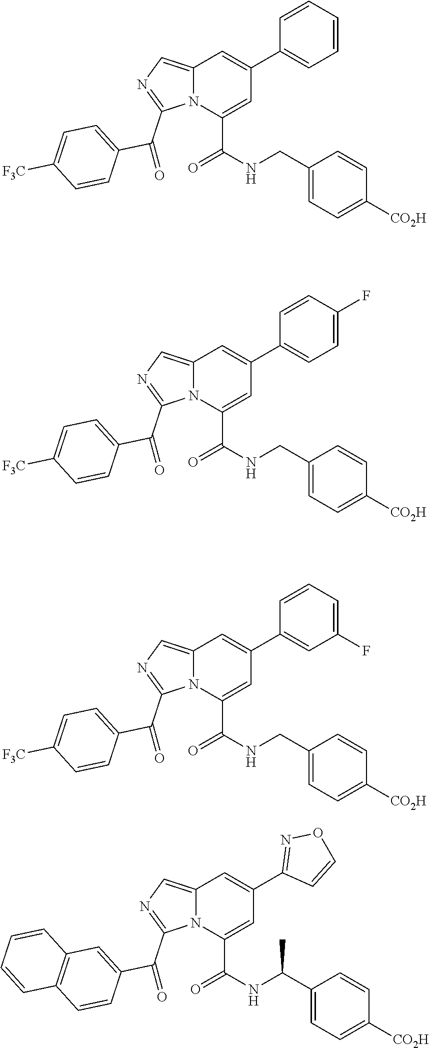

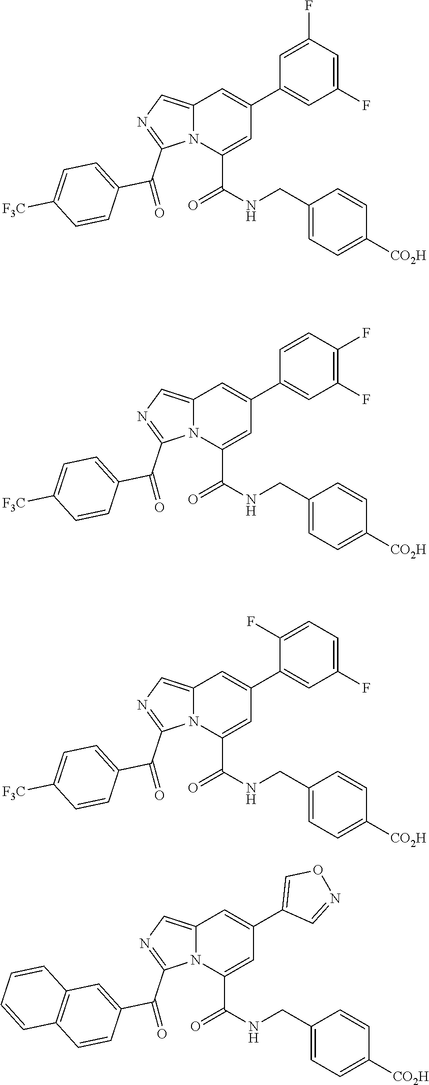

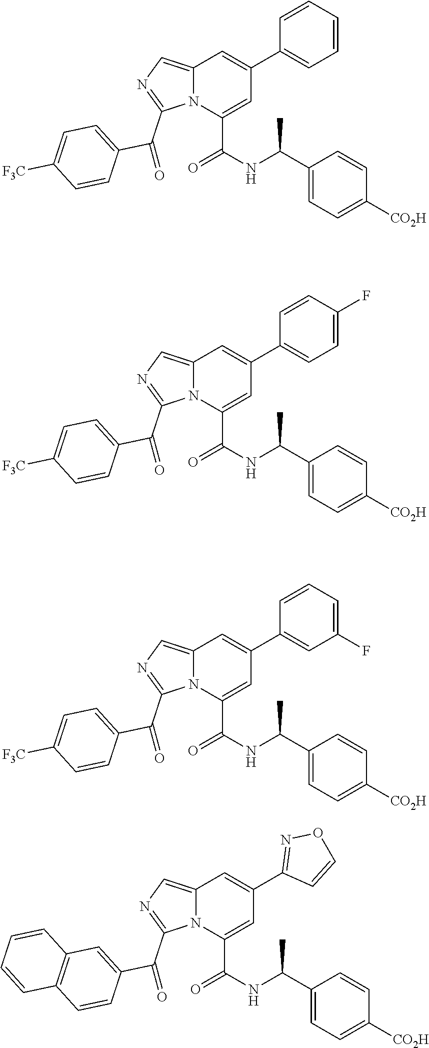

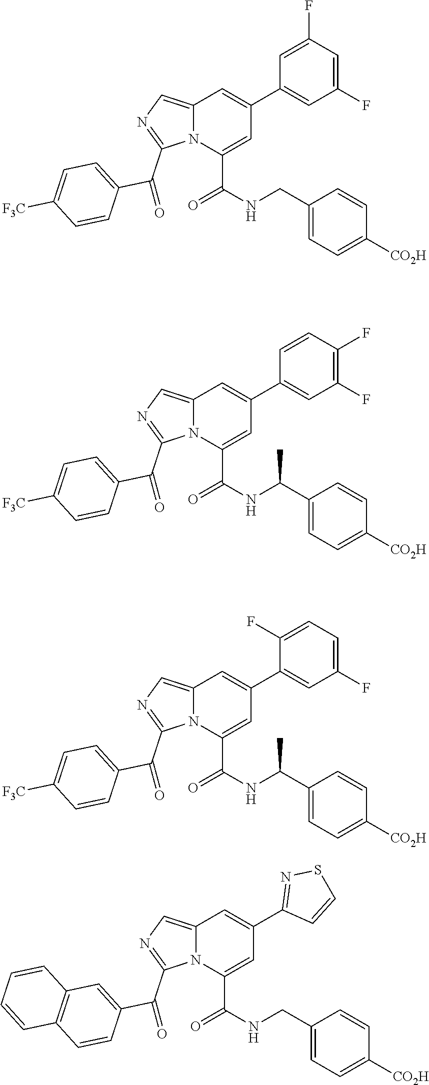

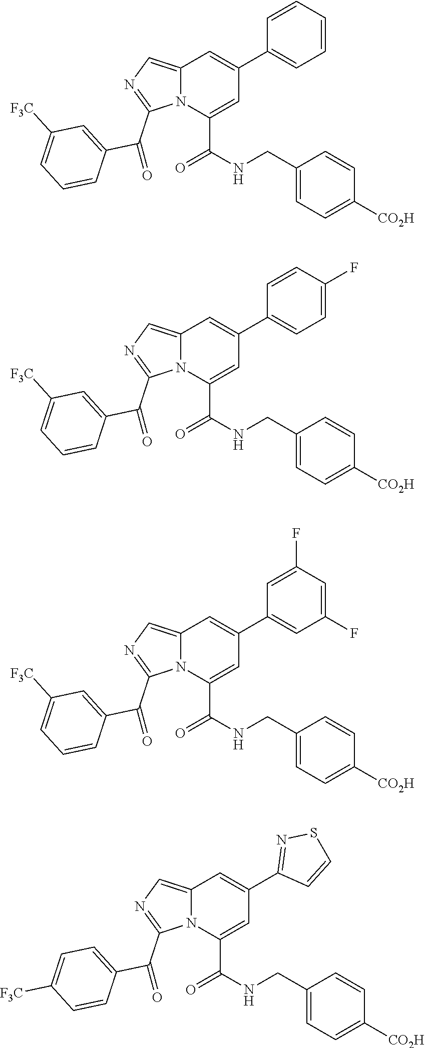 [SMILES: O=C(O)c1ccc(CNC(=O)c2cc(-c3ccccc3)cc3cnc(C(=O)c4cccc(C(F)(F)F)c4)n23)cc1 ; O=C(O)c1ccc(CNC(=O)c2cc(-c3ccc(F)cc3)cc3cnc(C(=O)c4cccc(C(F)(F)F)c4)n23)cc1 ; O=C(O)c1ccc(CNC(=O)c2cc(-c3cc(F)cc(F)c3)cc3cnc(C(=O)c4cccc(C(F)(F)F)c4)n23)cc1 ; O=C(O)c1ccc(CNC(=O)c2cc(-c3ccsn3)cc3cnc(C(=O)c4ccc(C(F)(F)F)cc4)n23)cc1]

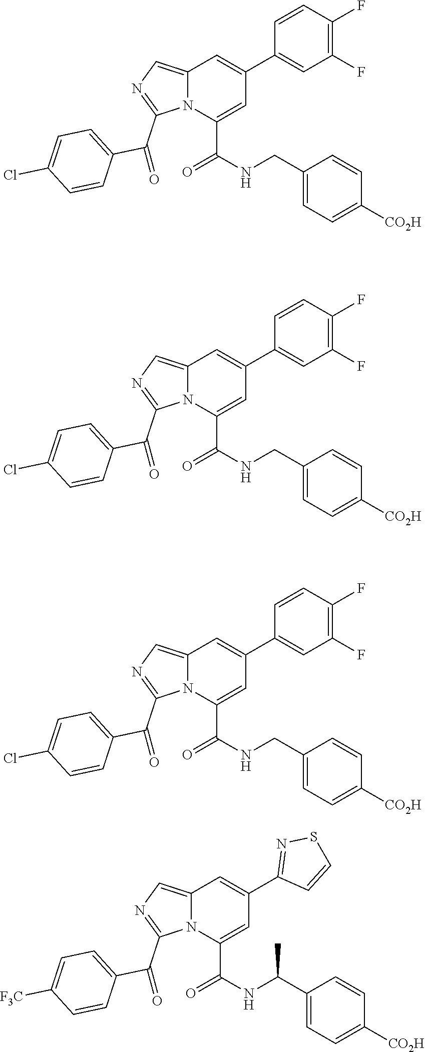

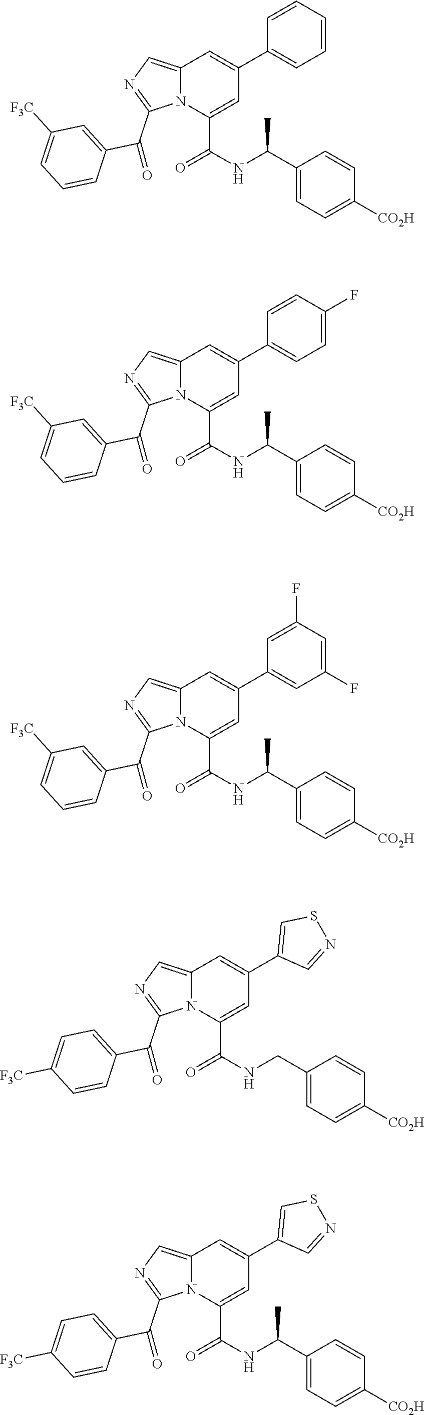 [SMILES: C[C@H](NC(=O)c1cc(-c2ccccc2)cc2cnc(C(=O)c3cccc(C(F)(F)F)c3)n12)c1ccc(C(=O)O)cc1 ; C[C@H](NC(=O)c1cc(-c2ccc(F)cc2)cc2cnc(C(=O)c3cccc(C(F)(F)F)c3)n12)c1ccc(C(=O)O)cc1 ; C[C@H](NC(=O)c1cc(-c2cc(F)cc(F)c2)cc2cnc(C(=O)c3cccc(C(F)(F)F)c3)n12)c1ccc(C(=O)O)cc1 ; O=C(O)c1ccc(CNC(=O)c2cc(-c3cnsc3)cc3cnc(C(=O)c4ccc(C(F)(F)F)cc4)n23)cc1 ; C[C@H](NC(=O)c1cc(-c2cnsc2)cc2cnc(C(=O)c3ccc(C(F)(F)F)cc3)n12)c1ccc(C(=O)O)cc1]

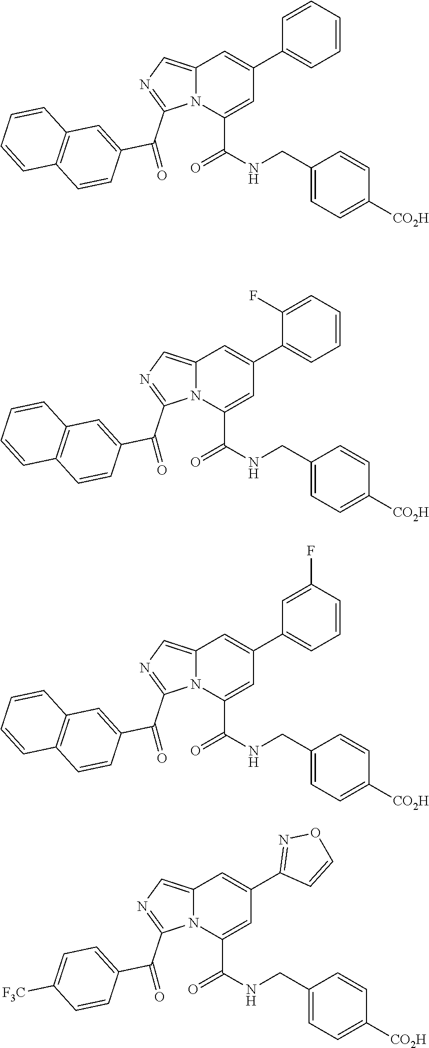

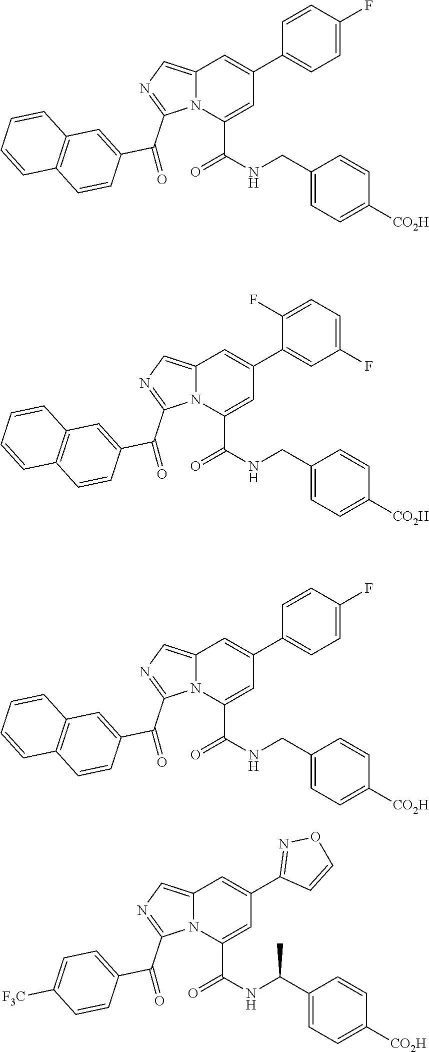

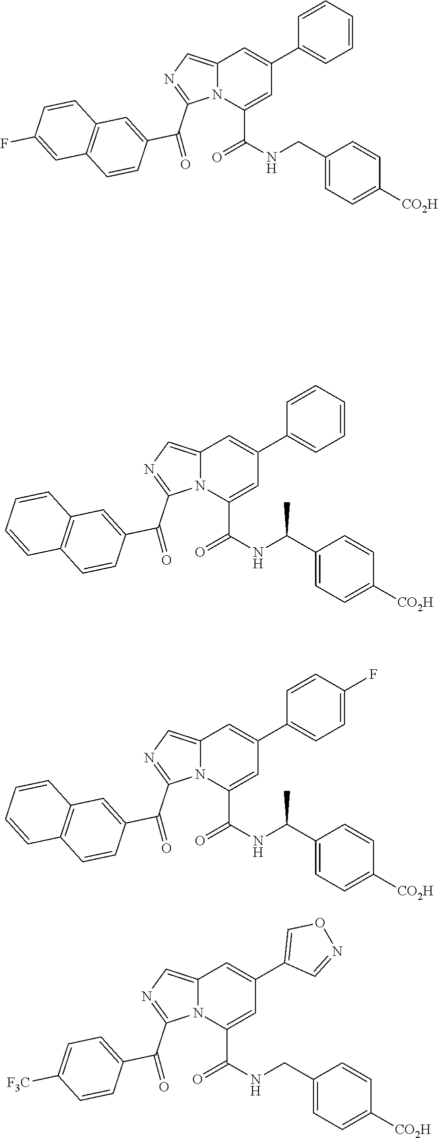

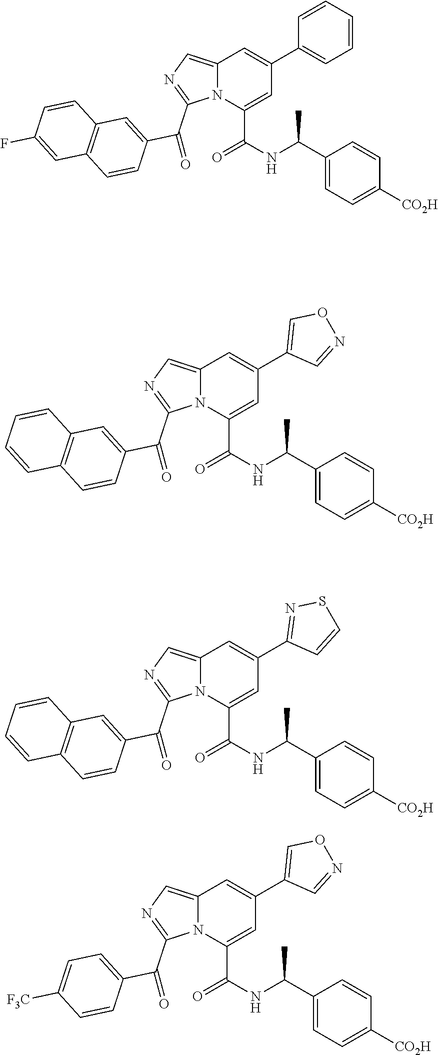

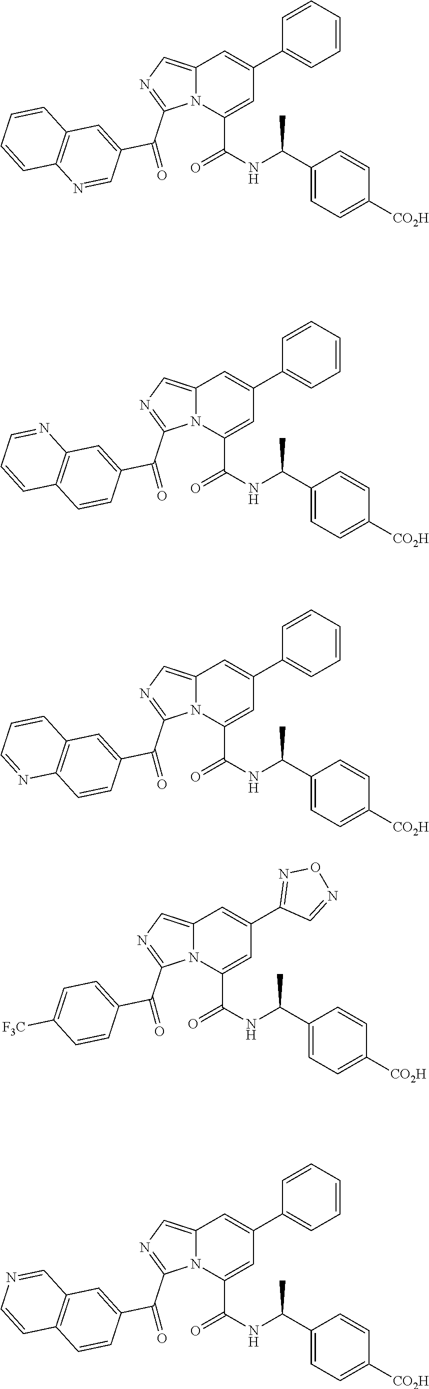 [SMILES: C[C@H](NC(=O)c1cc(-c2ccccc2)cc2cnc(C(=O)c3cnc4ccccc4c3)n12)c1ccc(C(=O)O)cc1 ; C[C@H](NC(=O)c1cc(-c2ccccc2)cc2cnc(C(=O)c3ccc4cccnc4c3)n12)c1ccc(C(=O)O)cc1 ; C[C@H](NC(=O)c1cc(-c2ccccc2)cc2cnc(C(=O)c3ccc4ncccc4c3)n12)c1ccc(C(=O)O)cc1 ; C[C@H](NC(=O)c1cc(-c2cnon2)cc2cnc(C(=O)c3ccc(C(F)(F)F)cc3)n12)c1ccc(C(=O)O)cc1 ; C[C@H](NC(=O)c1cc(-c2ccccc2)cc2cnc(C(=O)c3ccc4ccncc4c3)n12)c1ccc(C(=O)O)cc1]

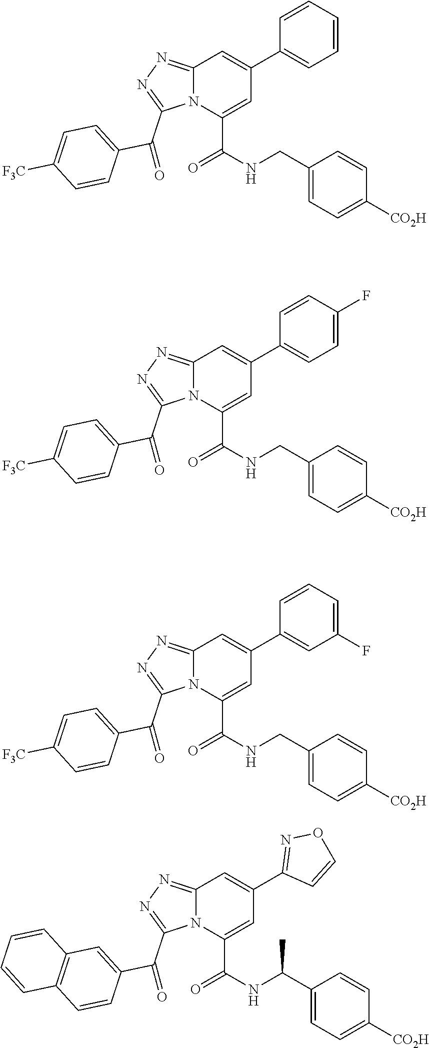

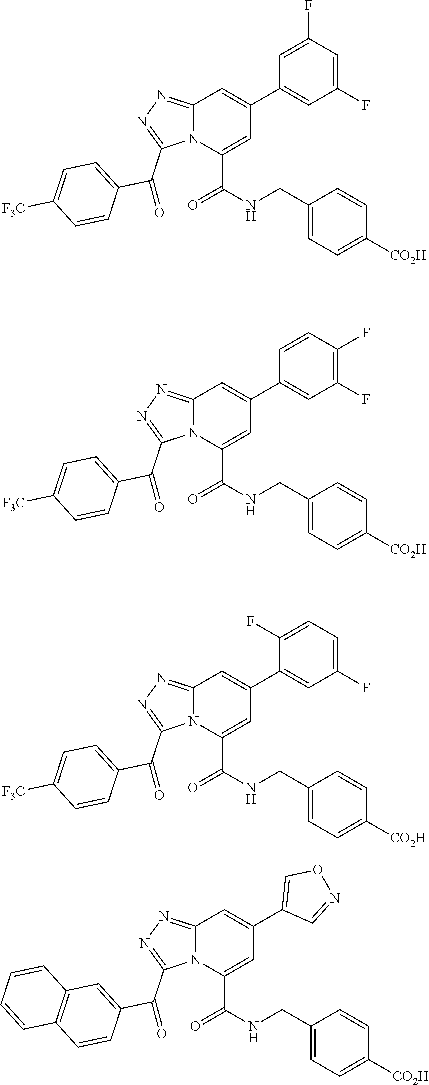

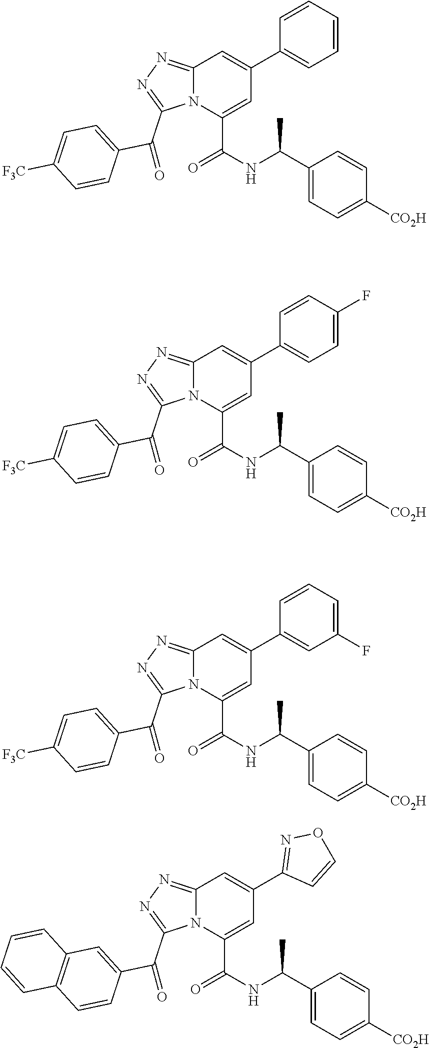

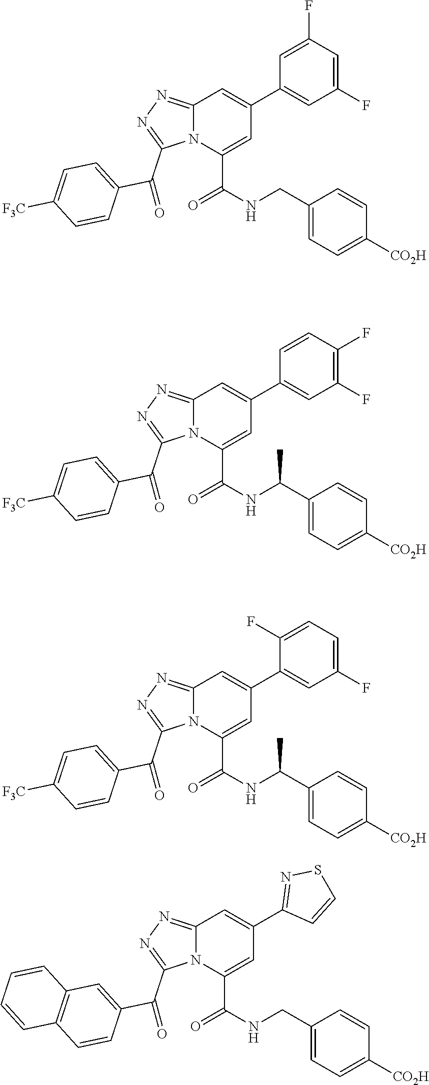

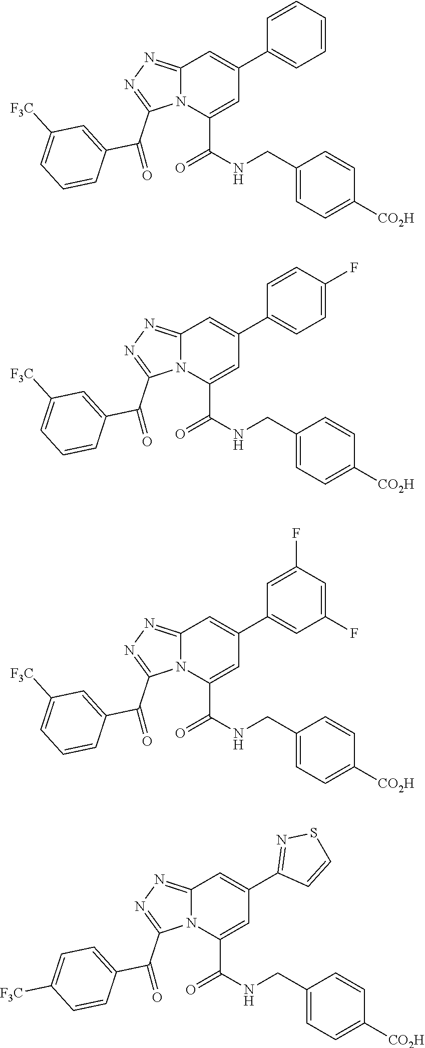 [SMILES: O=C(O)c1ccc(CNC(=O)c2cc(-c3ccccc3)cc3nnc(C(=O)c4cccc(C(F)(F)F)c4)n23)cc1 ; O=C(O)c1ccc(CNC(=O)c2cc(-c3ccc(F)cc3)cc3nnc(C(=O)c4cccc(C(F)(F)F)c4)n23)cc1 ; O=C(O)c1ccc(CNC(=O)c2cc(-c3cc(F)cc(F)c3)cc3nnc(C(=O)c4cccc(C(F)(F)F)c4)n23)cc1 ; O=C(O)c1ccc(CNC(=O)c2cc(-c3ccsn3)cc3nnc(C(=O)c4ccc(C(F)(F)F)cc4)n23)cc1]

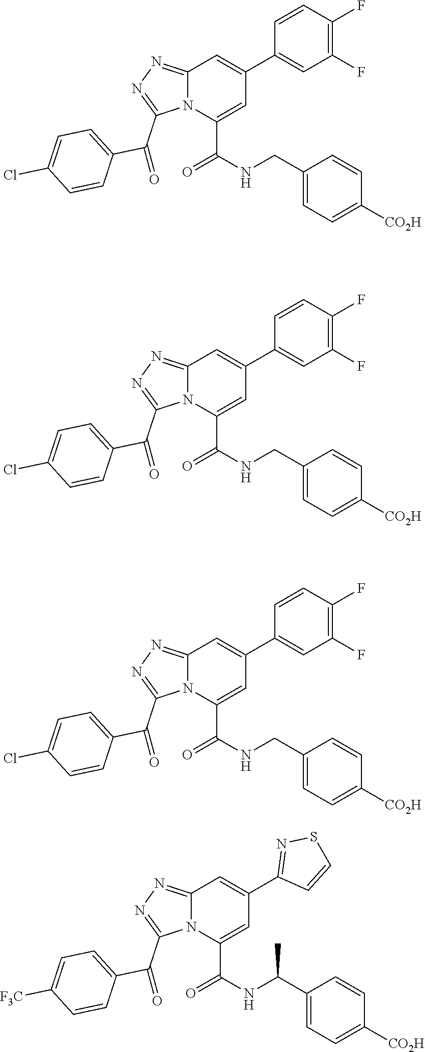

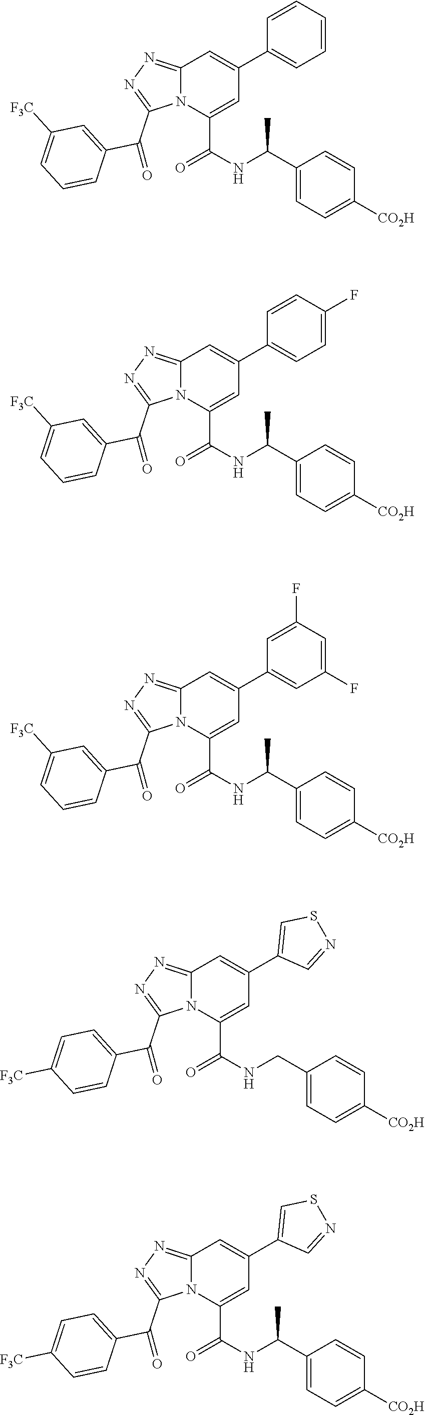 [SMILES: C[C@H](NC(=O)c1cc(-c2ccccc2)cc2nnc(C(=O)c3cccc(C(F)(F)F)c3)n12)c1ccc(C(=O)O)cc1 ; C[C@H](NC(=O)c1cc(-c2ccc(F)cc2)cc2nnc(C(=O)c3cccc(C(F)(F)F)c3)n12)c1ccc(C(=O)O)cc1 ; C[C@H](NC(=O)c1cc(-c2cc(F)cc(F)c2)cc2nnc(C(=O)c3cccc(C(F)(F)F)c3)n12)c1ccc(C(=O)O)cc1 ; O=C(O)c1ccc(CNC(=O)c2cc(-c3cnsc3)cc3nnc(C(=O)c4ccc(C(F)(F)F)cc4)n23)cc1 ; C[C@H](NC(=O)c1cc(-c2cnsc2)cc2nnc(C(=O)c3ccc(C(F)(F)F)cc3)n12)c1ccc(C(=O)O)cc1]

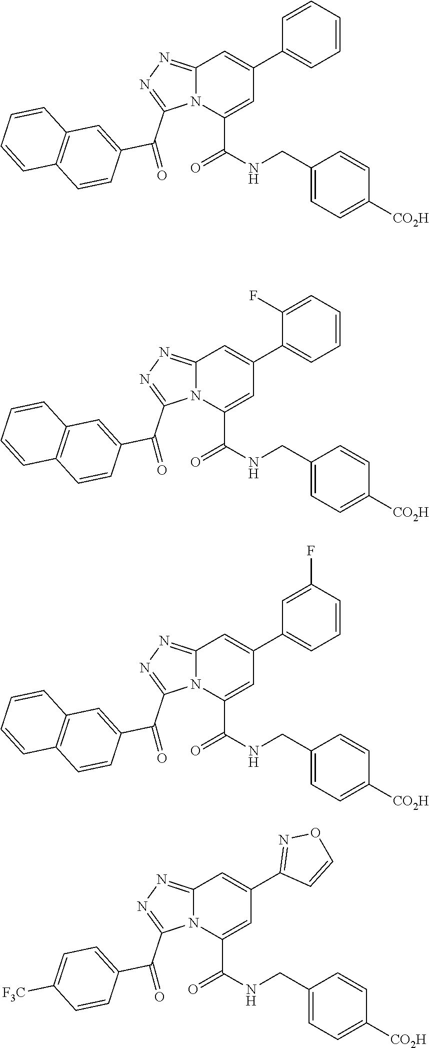

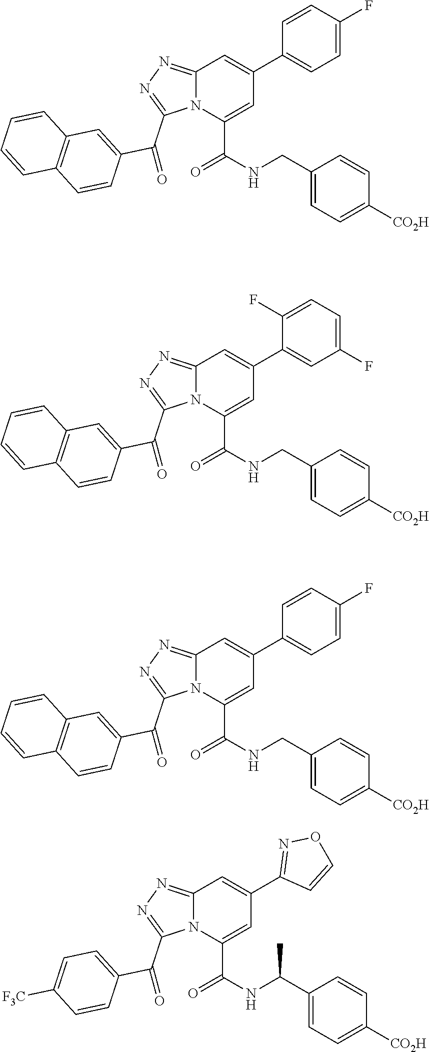

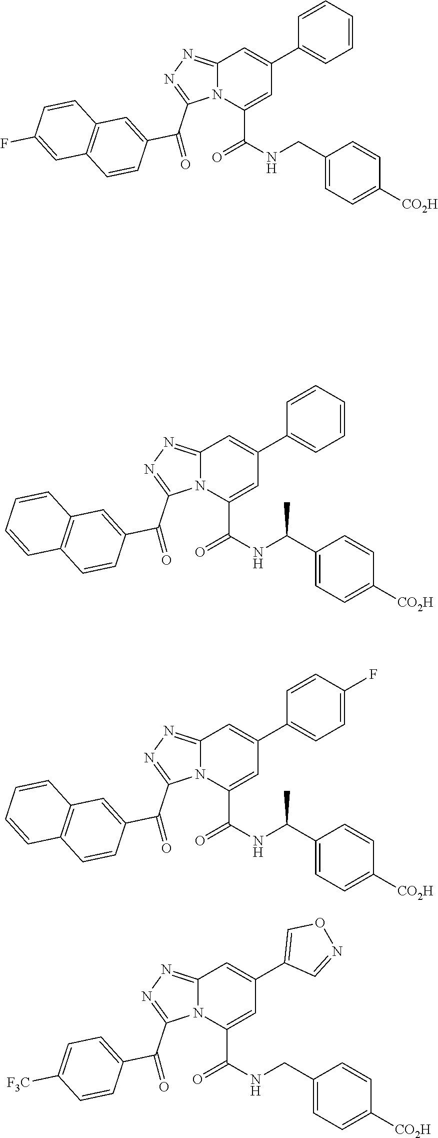

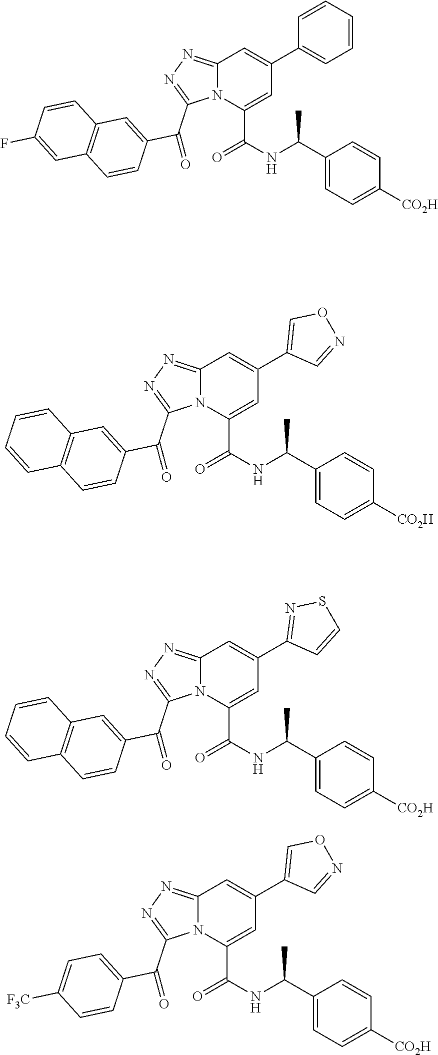

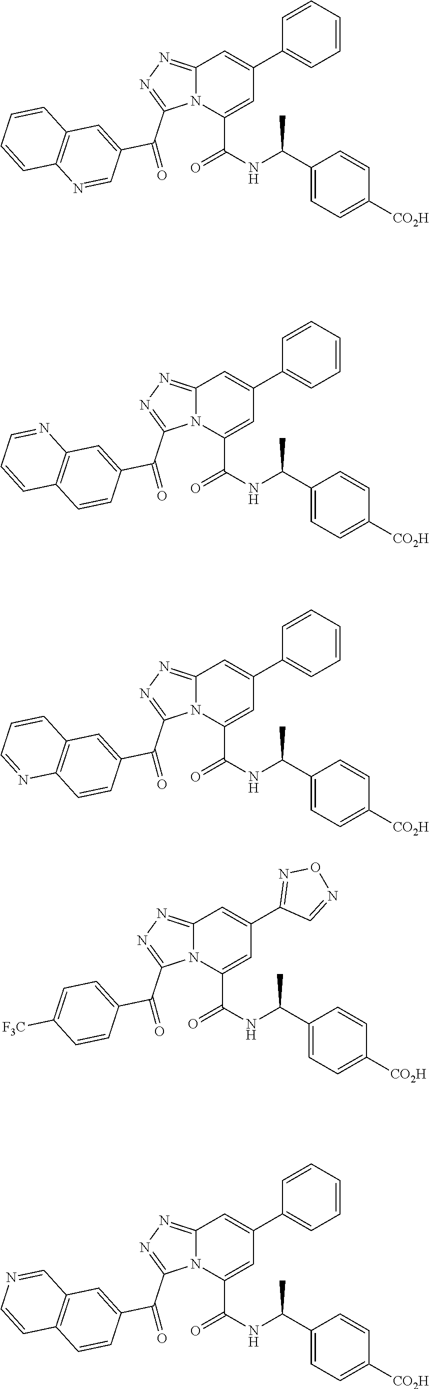 [SMILES: C[C@H](NC(=O)c1cc(-c2ccccc2)cc2nnc(C(=O)c3cnc4ccccc4c3)n12)c1ccc(C(=O)O)cc1 ; C[C@H](NC(=O)c1cc(-c2ccccc2)cc2nnc(C(=O)c3ccc4cccnc4c3)n12)c1ccc(C(=O)O)cc1 ; C[C@H](NC(=O)c1cc(-c2ccccc2)cc2nnc(C(=O)c3ccc4ncccc4c3)n12)c1ccc(C(=O)O)cc1 ; C[C@H](NC(=O)c1cc(-c2cnon2)cc2nnc(C(=O)c3ccc(C(F)(F)F)cc3)n12)c1ccc(C(=O)O)cc1 ; C[C@H](NC(=O)c1cc(-c2ccccc2)cc2nnc(C(=O)c3ccc4ccncc4c3)n12)c1ccc(C(=O)O)cc1]

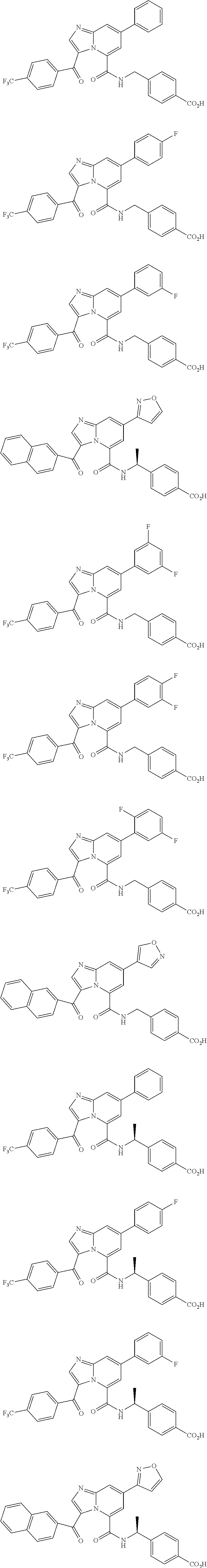

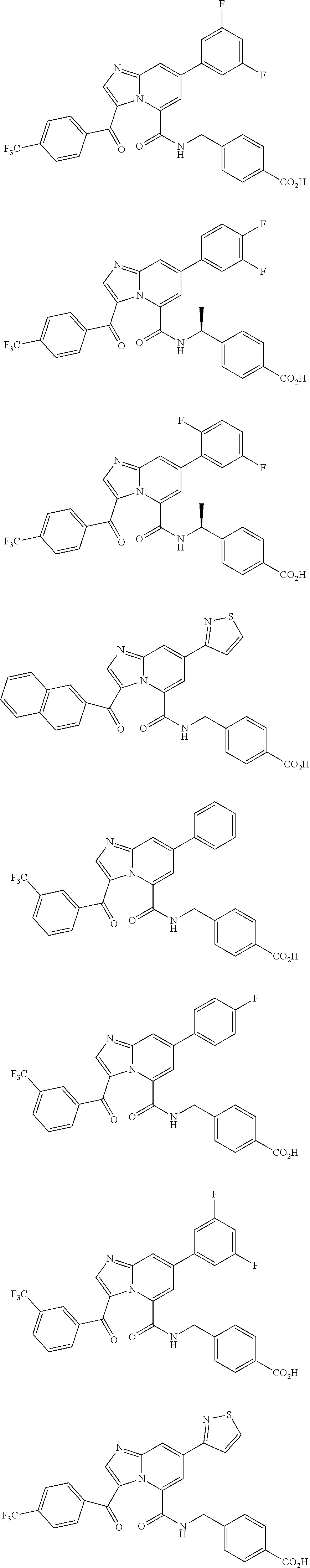

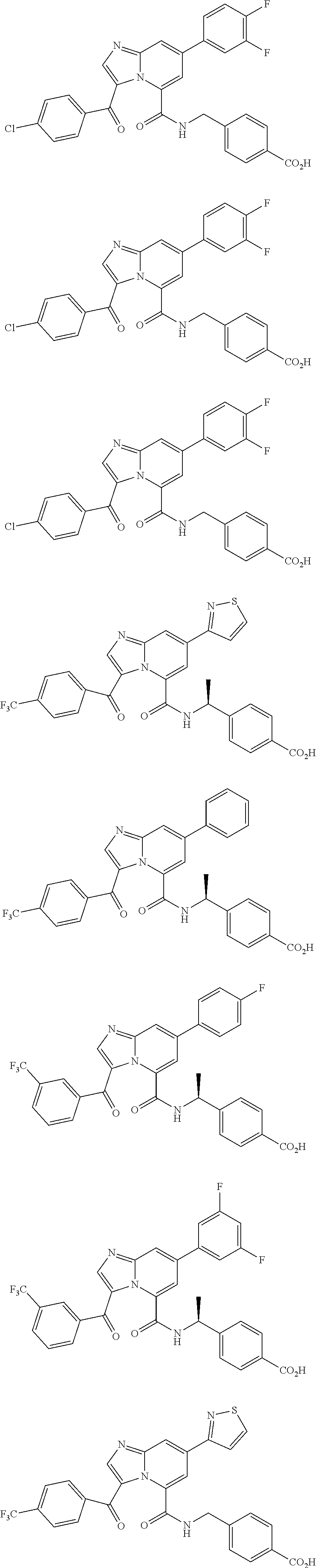

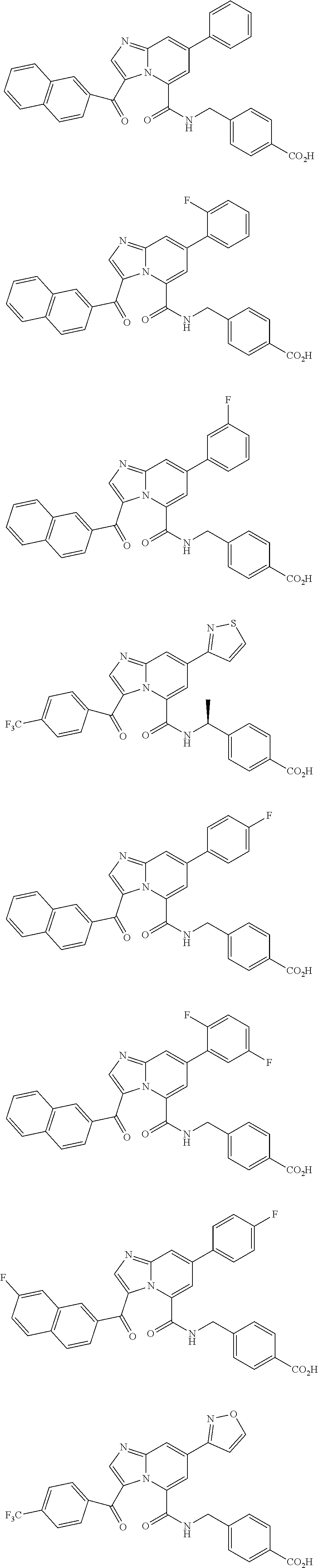

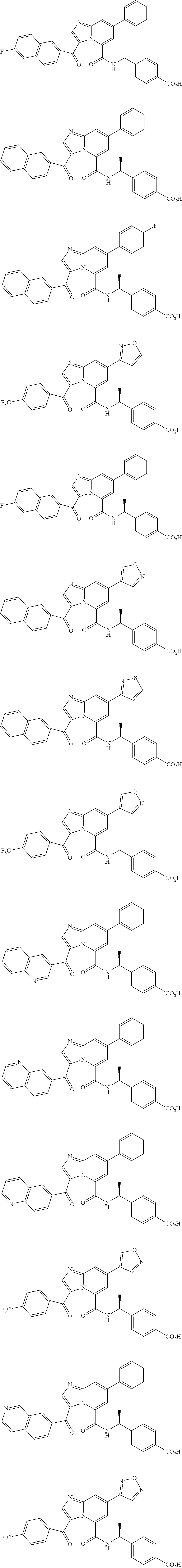

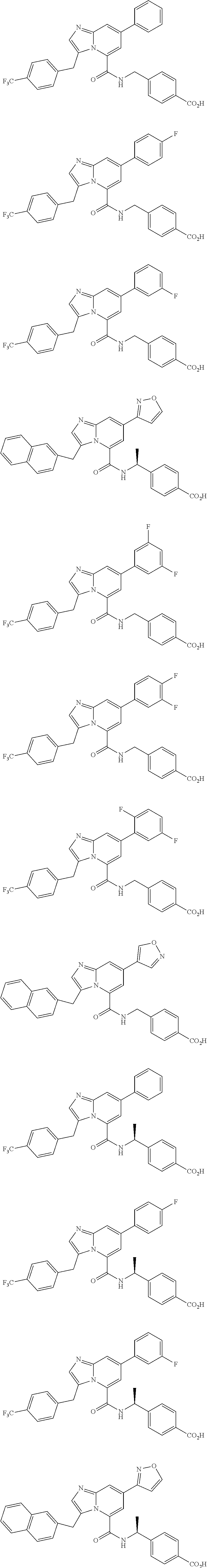

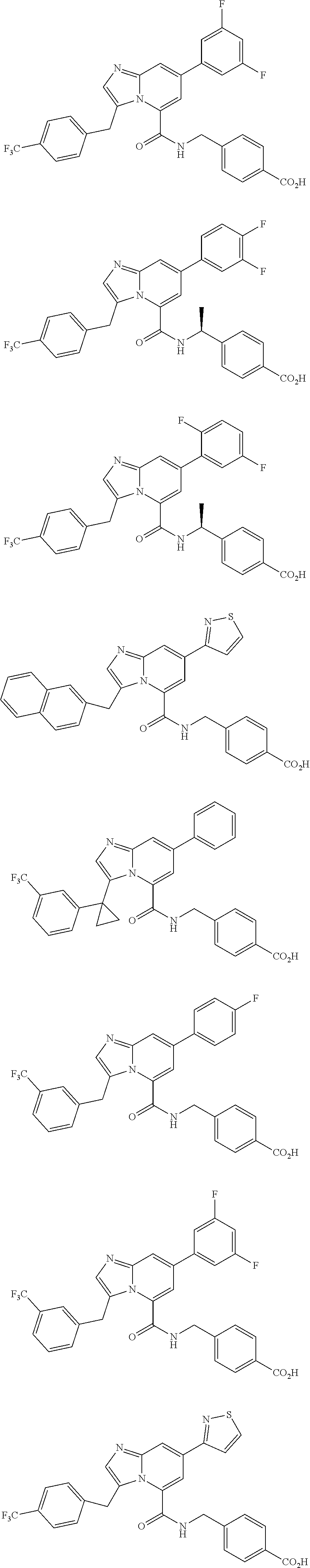

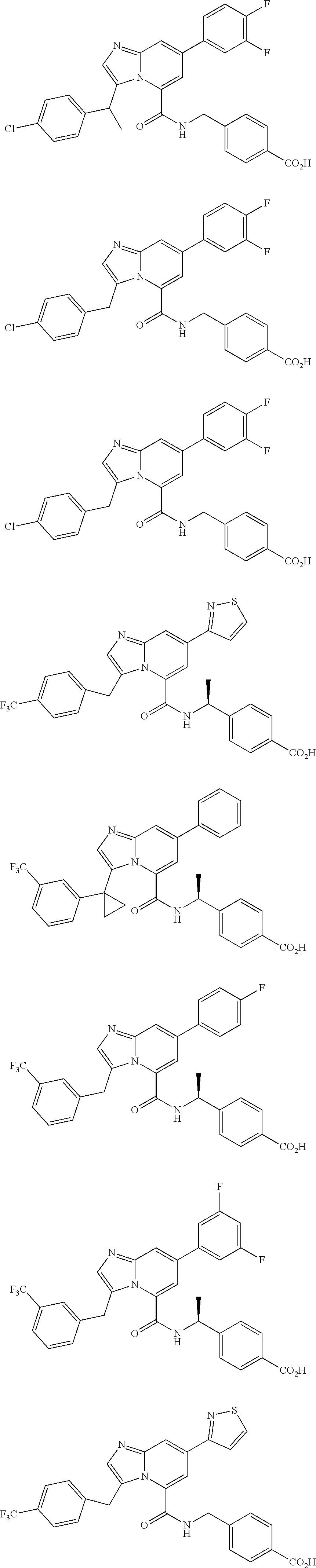

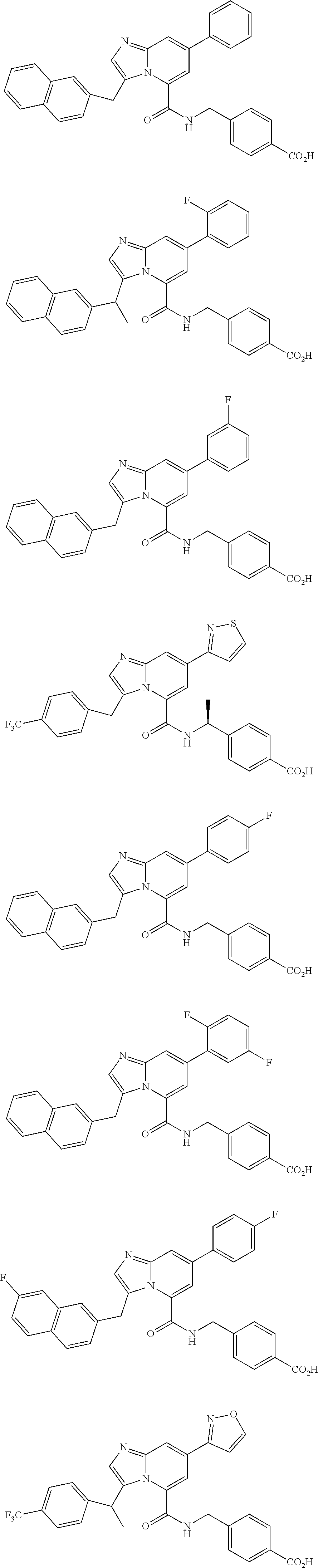

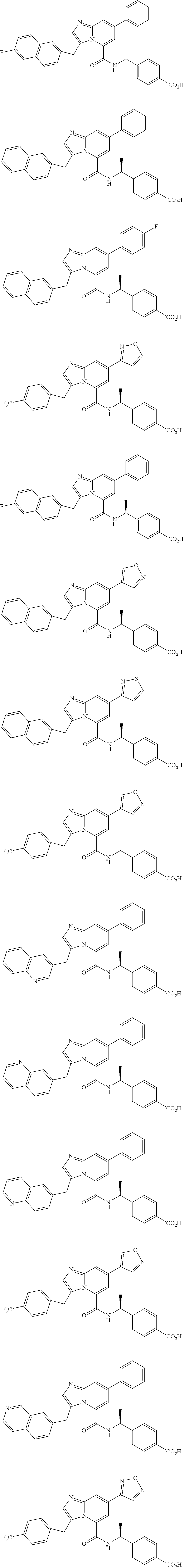

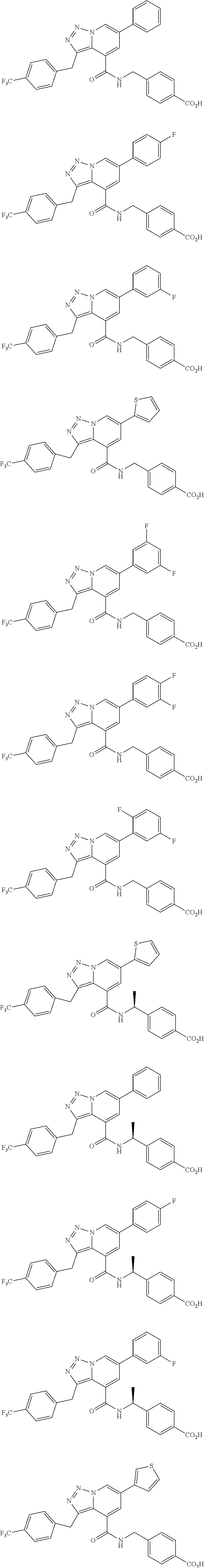

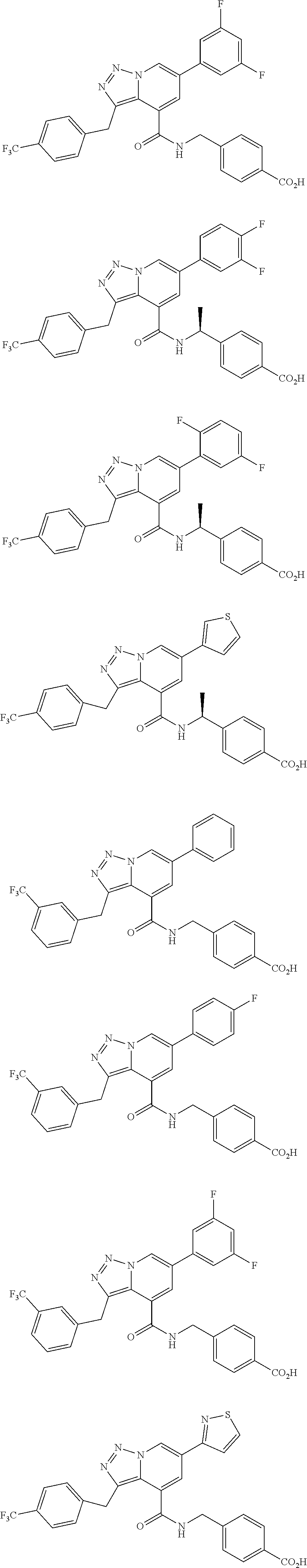

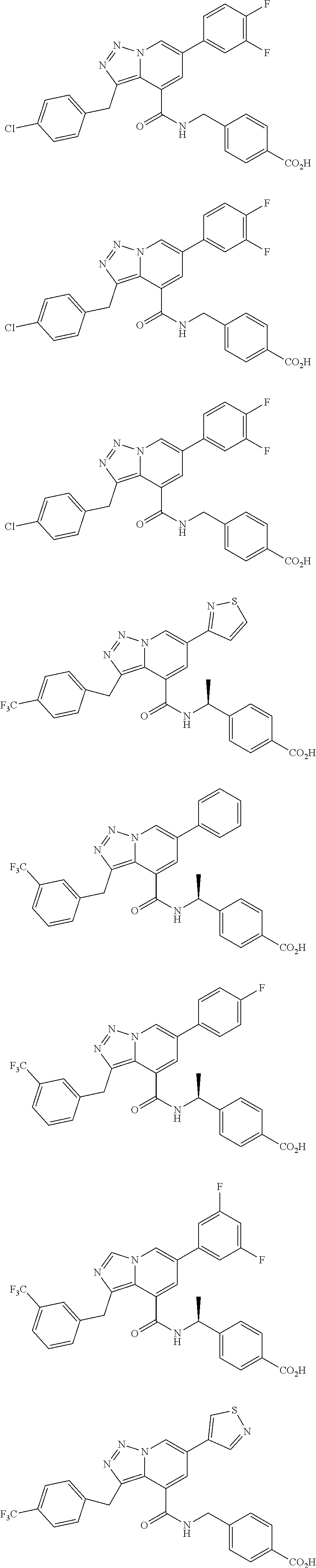

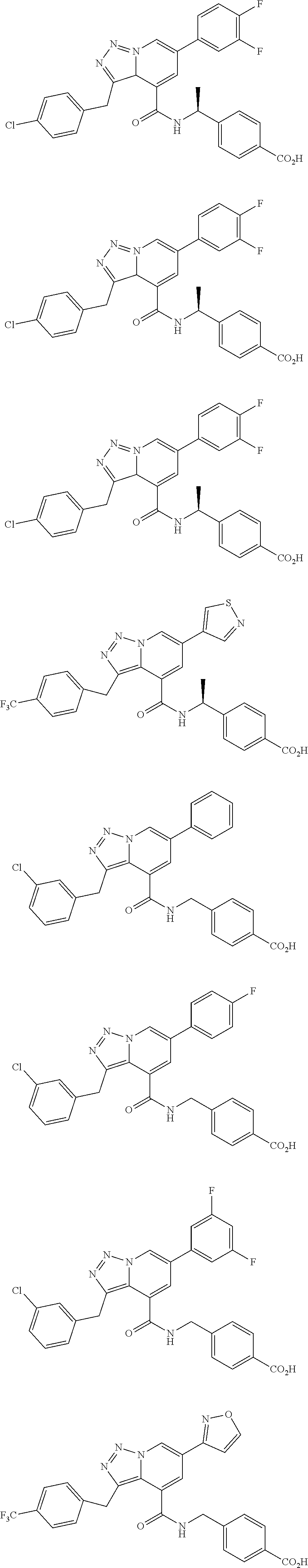

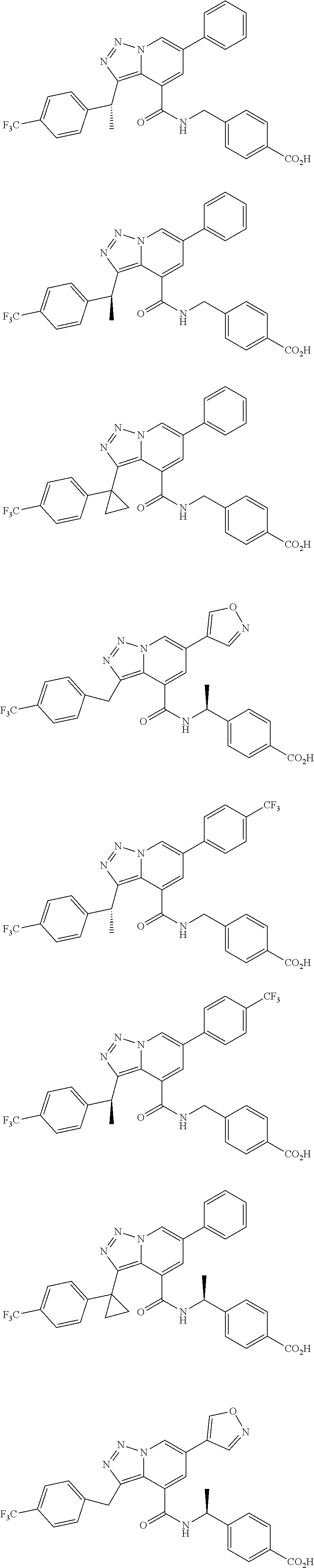

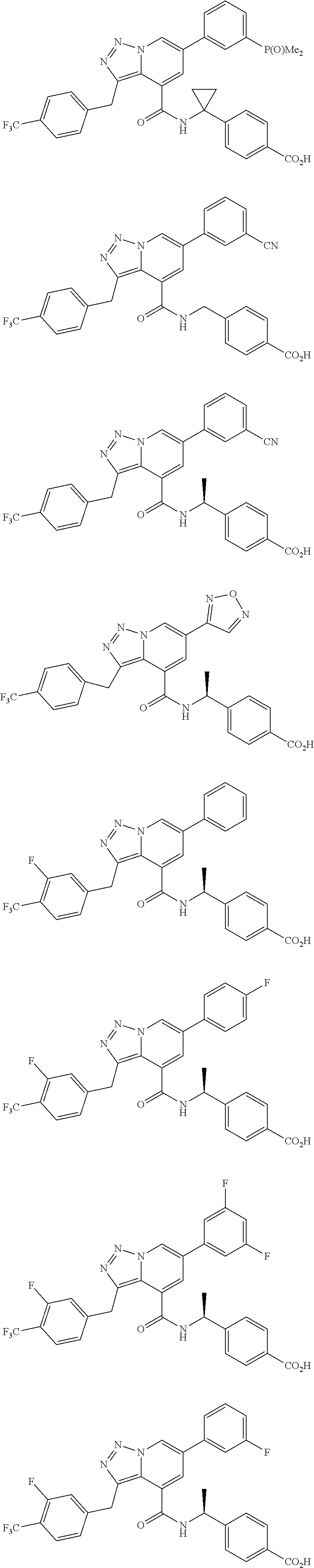

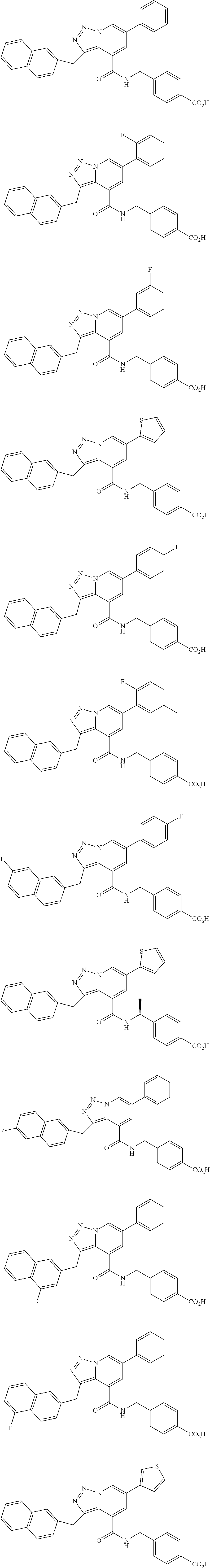

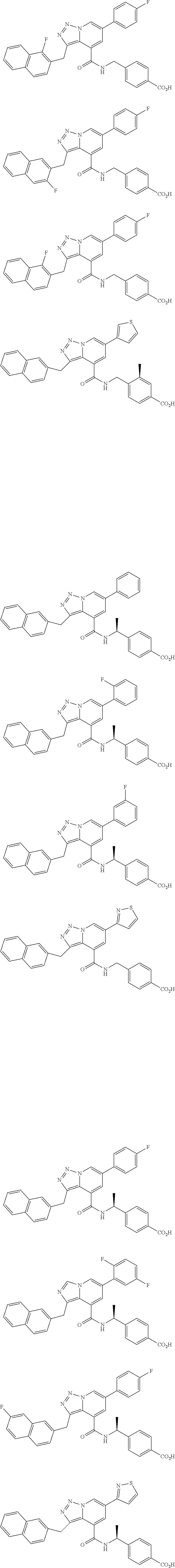

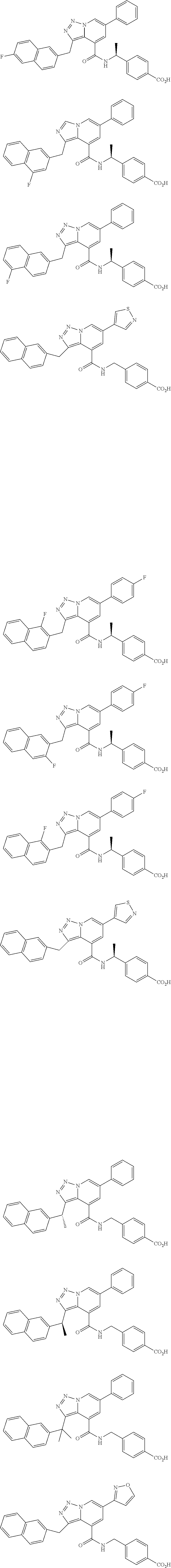

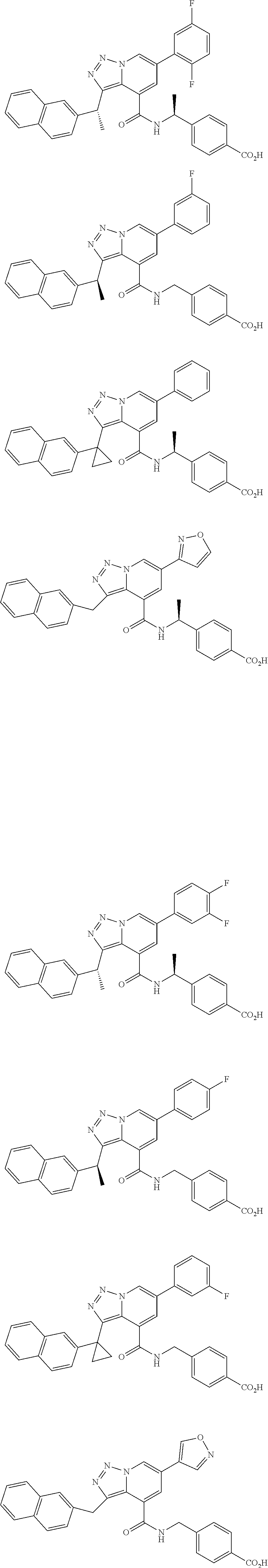

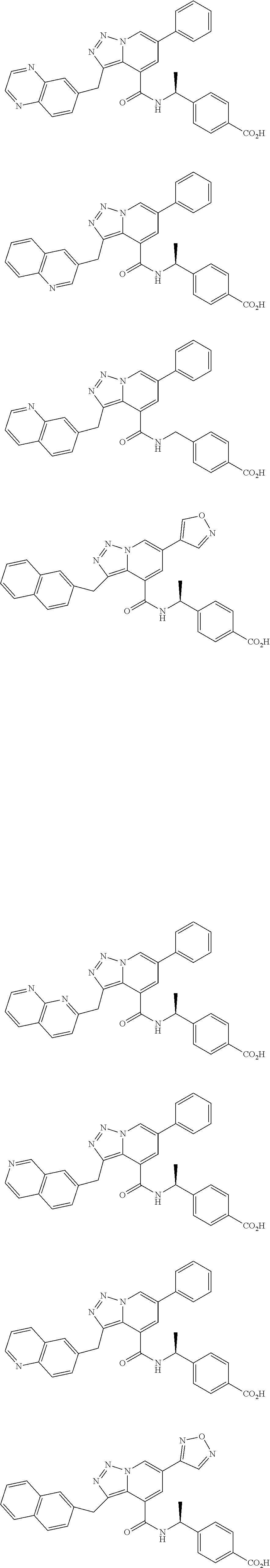

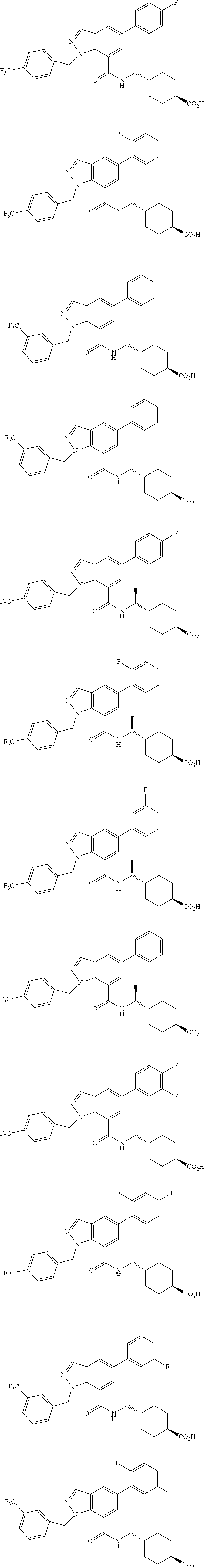

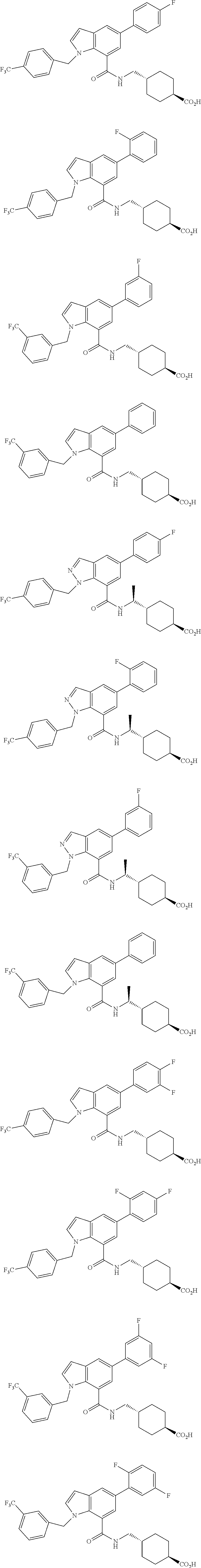

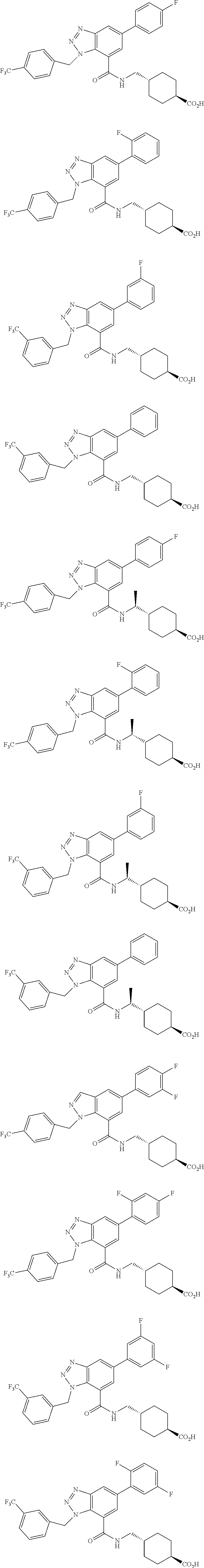

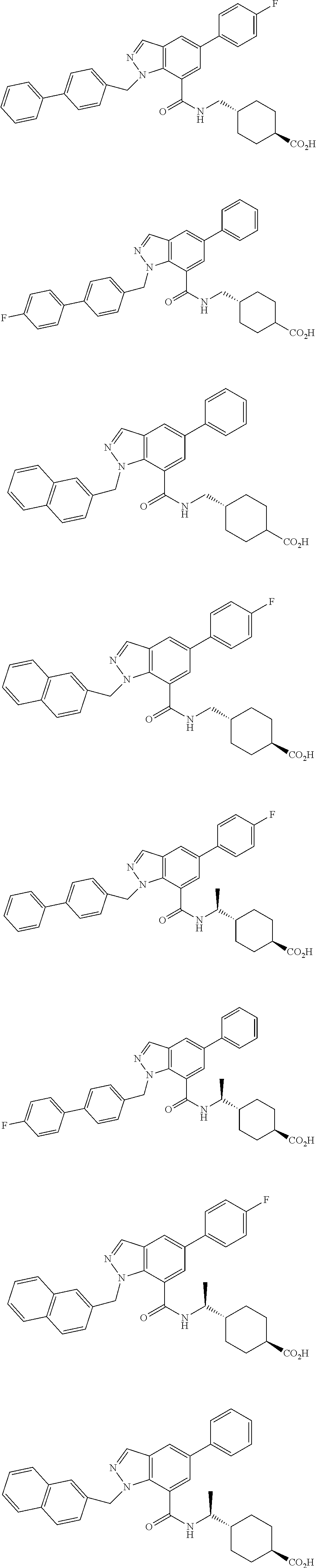

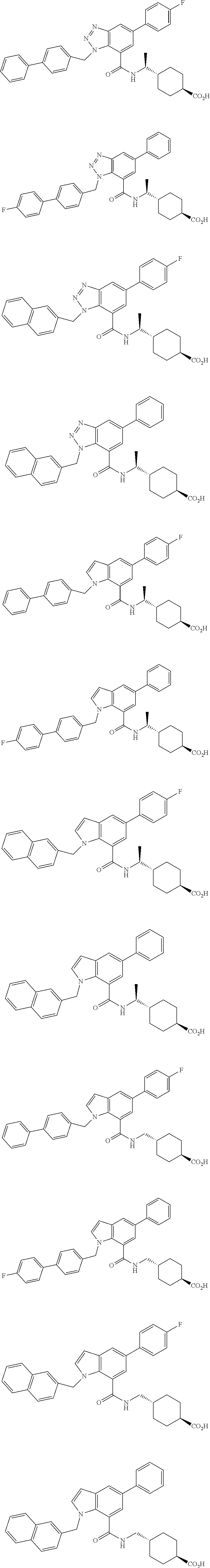

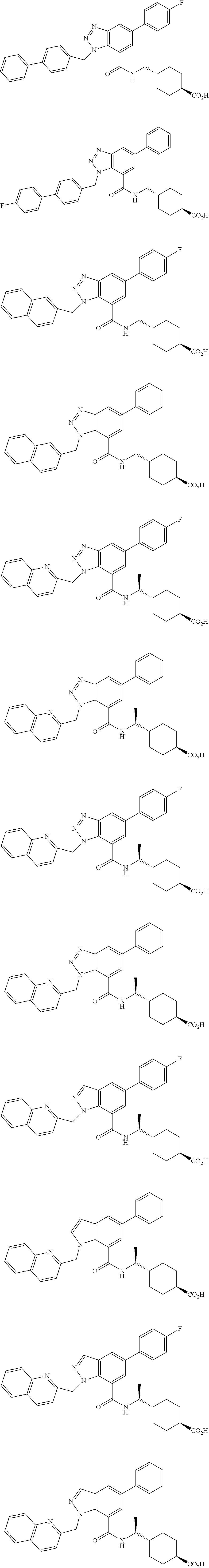

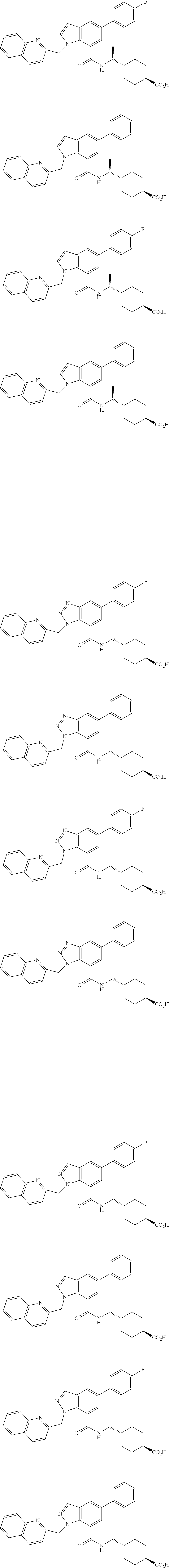

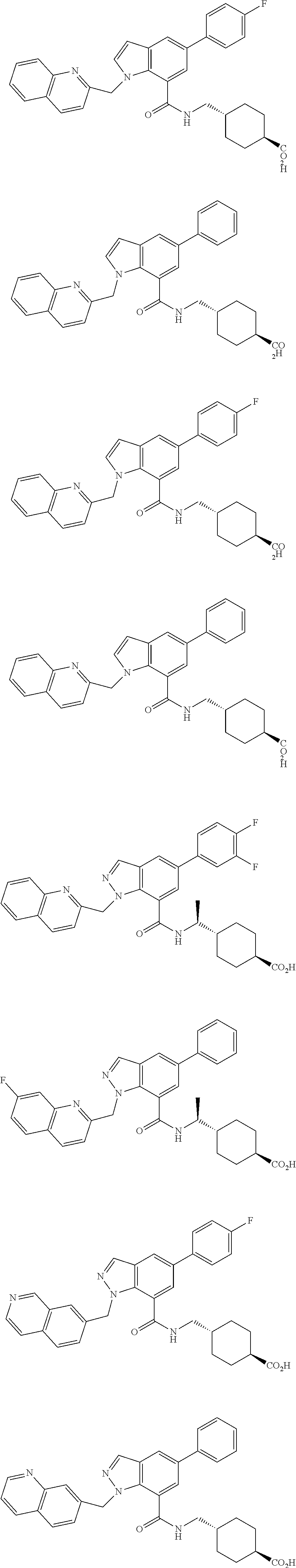

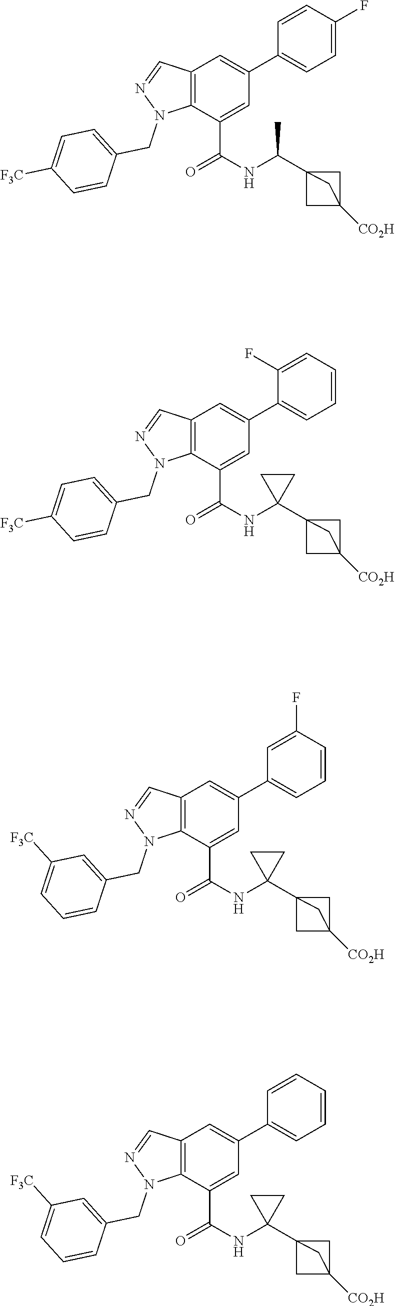 [SMILES: C[C@H](NC(=O)c1cc(-c2ccc(F)cc2)cc2cnn(Cc3ccc(C(F)(F)F)cc3)c12)C12CC(C(=O)O)(C1)C2 ; O=C(NC1(C23CC(C(=O)O)(C2)C3)CC1)c1cc(-c2ccccc2F)cc2cnn(Cc3ccc(C(F)(F)F)cc3)c12 ; O=C(NC1(C23CC(C(=O)O)(C2)C3)CC1)c1cc(-c2cccc(F)c2)cc2cnn(Cc3cccc(C(F)(F)F)c3)c12 ; O=C(NC1(C23CC(C(=O)O)(C2)C3)CC1)c1cc(-c2ccccc2)cc2cnn(Cc3cccc(C(F)(F)F)c3)c12]

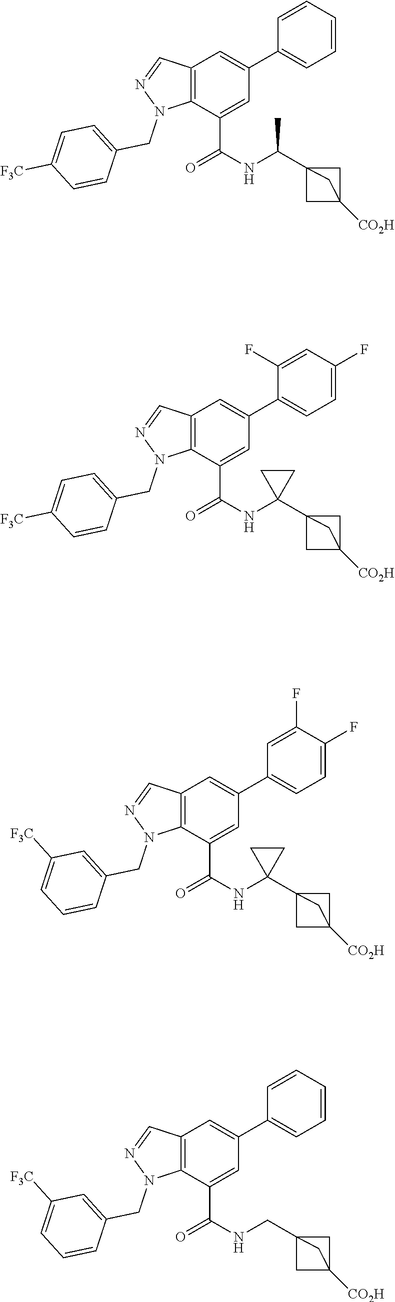 [SMILES: C[C@H](NC(=O)c1cc(-c2ccccc2)cc2cnn(Cc3ccc(C(F)(F)F)cc3)c12)C12CC(C(=O)O)(C1)C2 ; O=C(NC1(C23CC(C(=O)O)(C2)C3)CC1)c1cc(-c2ccc(F)cc2F)cc2cnn(Cc3ccc(C(F)(F)F)cc3)c12 ; O=C(NC1(C23CC(C(=O)O)(C2)C3)CC1)c1cc(-c2ccc(F)c(F)c2)cc2cnn(Cc3cccc(C(F)(F)F)c3)c12 ; O=C(NCC12CC(C(=O)O)(C1)C2)c1cc(-c2ccccc2)cc2cnn(Cc3cccc(C(F)(F)F)c3)c12]

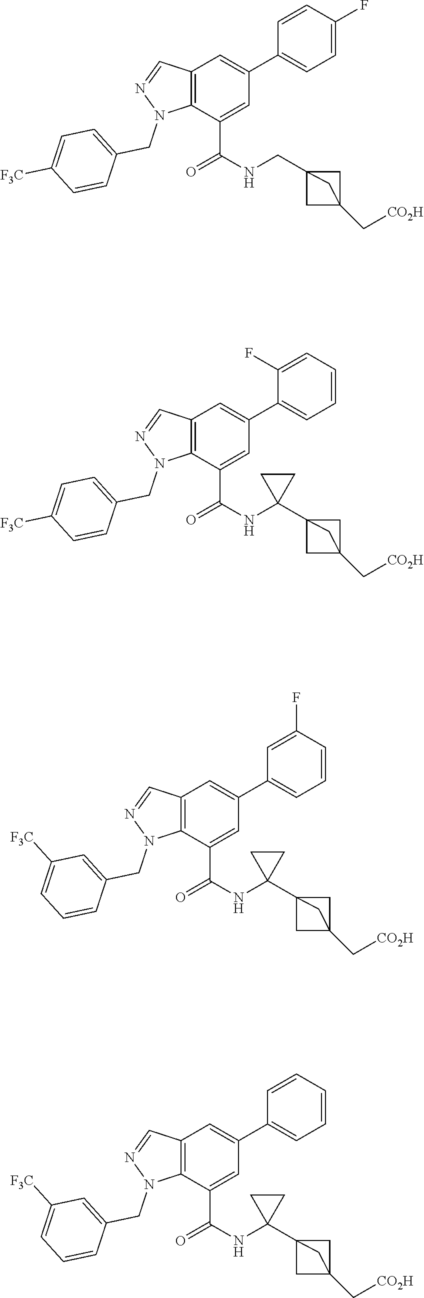 [SMILES: O=C(O)CC12CC(CNC(=O)c3cc(-c4ccc(F)cc4)cc4cnn(Cc5ccc(C(F)(F)F)cc5)c34)(C1)C2 ; O=C(O)CC12CC(C3(NC(=O)c4cc(-c5ccccc5F)cc5cnn(Cc6ccc(C(F)(F)F)cc6)c45)CC3)(C1)C2 ; O=C(O)CC12CC(C3(NC(=O)c4cc(-c5cccc(F)c5)cc5cnn(Cc6cccc(C(F)(F)F)c6)c45)CC3)(C1)C2 ; O=C(O)CC12CC(C3(NC(=O)c4cc(-c5ccccc5)cc5cnn(Cc6cccc(C(F)(F)F)c6)c45)CC3)(C1)C2]

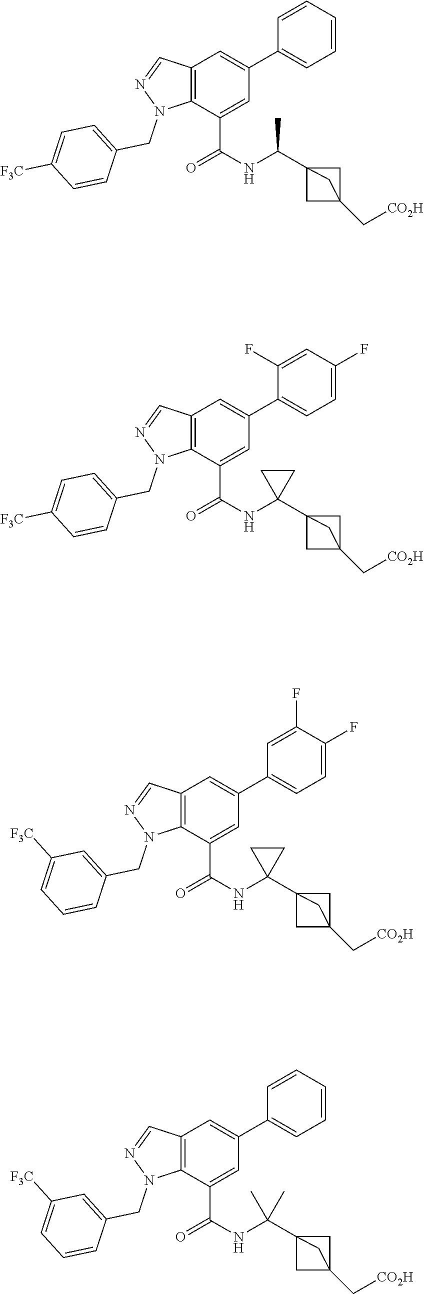 [SMILES: C[C@H](NC(=O)c1cc(-c2ccccc2)cc2cnn(Cc3ccc(C(F)(F)F)cc3)c12)C12CC(CC(=O)O)(C1)C2 ; O=C(O)CC12CC(C3(NC(=O)c4cc(-c5ccc(F)cc5F)cc5cnn(Cc6ccc(C(F)(F)F)cc6)c45)CC3)(C1)C2 ; O=C(O)CC12CC(C3(NC(=O)c4cc(-c5ccc(F)c(F)c5)cc5cnn(Cc6cccc(C(F)(F)F)c6)c45)CC3)(C1)C2 ; CC(C)(NC(=O)c1cc(-c2ccccc2)cc2cnn(Cc3cccc(C(F)(F)F)c3)c12)C12CC(CC(=O)O)(C1)C2]

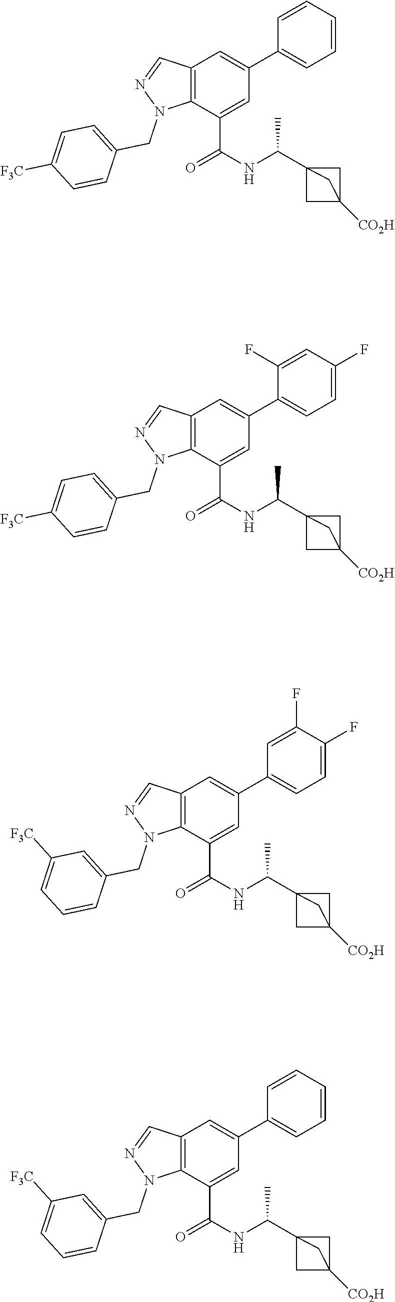 [SMILES: C[C@@H](NC(=O)c1cc(-c2ccccc2)cc2cnn(Cc3ccc(C(F)(F)F)cc3)c12)C12CC(C(=O)O)(C1)C2 ; C[C@H](NC(=O)c1cc(-c2ccc(F)cc2F)cc2cnn(Cc3ccc(C(F)(F)F)cc3)c12)C12CC(C(=O)O)(C1)C2 ; C[C@@H](NC(=O)c1cc(-c2ccc(F)c(F)c2)cc2cnn(Cc3cccc(C(F)(F)F)c3)c12)C12CC(C(=O)O)(C1)C2 ; C[C@@H](NC(=O)c1cc(-c2ccccc2)cc2cnn(Cc3cccc(C(F)(F)F)c3)c12)C12CC(C(=O)O)(C1)C2]

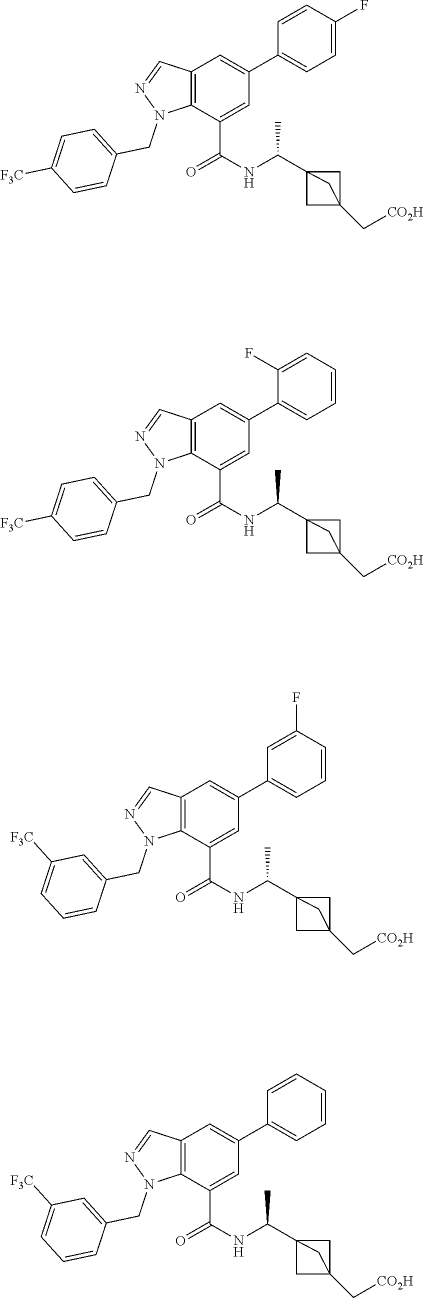 [SMILES: C[C@@H](NC(=O)c1cc(-c2ccc(F)cc2)cc2cnn(Cc3ccc(C(F)(F)F)cc3)c12)C12CC(CC(=O)O)(C1)C2 ; C[C@H](NC(=O)c1cc(-c2ccccc2F)cc2cnn(Cc3ccc(C(F)(F)F)cc3)c12)C12CC(CC(=O)O)(C1)C2 ; C[C@@H](NC(=O)c1cc(-c2cccc(F)c2)cc2cnn(Cc3cccc(C(F)(F)F)c3)c12)C12CC(CC(=O)O)(C1)C2 ; C[C@H](NC(=O)c1cc(-c2ccccc2)cc2cnn(Cc3cccc(C(F)(F)F)c3)c12)C12CC(CC(=O)O)(C1)C2]

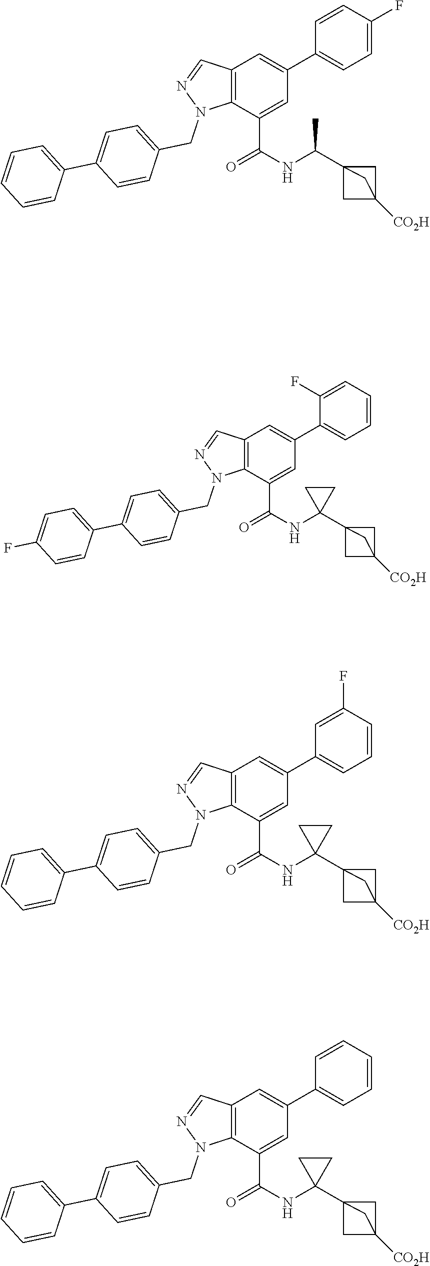 [SMILES: C[C@H](NC(=O)c1cc(-c2ccc(F)cc2)cc2cnn(Cc3ccc(-c4ccccc4)cc3)c12)C12CC(C(=O)O)(C1)C2 ; O=C(NC1(C23CC(C(=O)O)(C2)C3)CC1)c1cc(-c2ccccc2F)cc2cnn(Cc3ccc(-c4ccc(F)cc4)cc3)c12 ; O=C(NC1(C23CC(C(=O)O)(C2)C3)CC1)c1cc(-c2cccc(F)c2)cc2cnn(Cc3ccc(-c4ccccc4)cc3)c12 ; O=C(NC1(C23CC(C(=O)O)(C2)C3)CC1)c1cc(-c2ccccc2)cc2cnn(Cc3ccc(-c4ccccc4)cc3)c12]

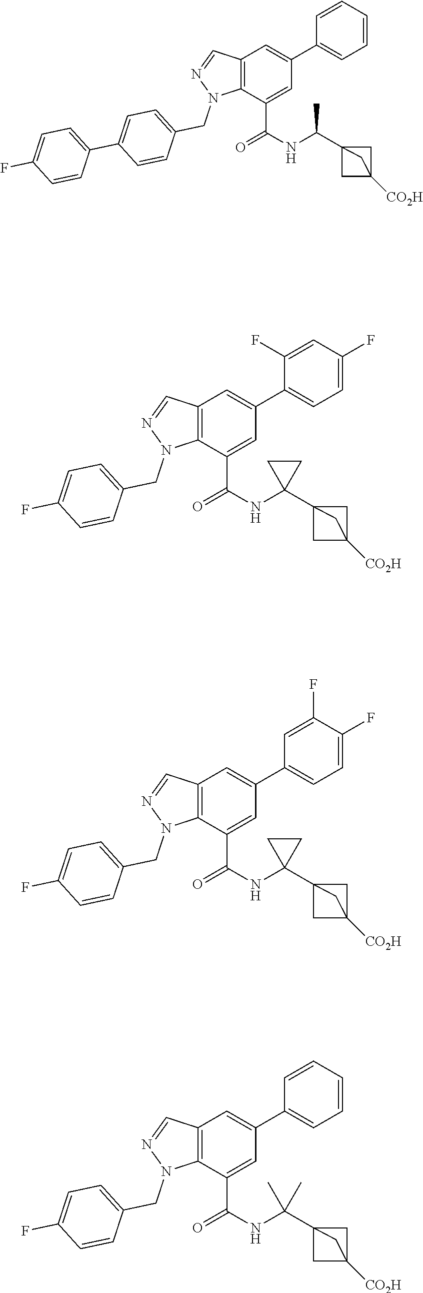 [SMILES: C[C@H](NC(=O)c1cc(-c2ccccc2)cc2cnn(Cc3ccc(-c4ccc(F)cc4)cc3)c12)C12CC(C(=O)O)(C1)C2 ; O=C(NC1(C23CC(C(=O)O)(C2)C3)CC1)c1cc(-c2ccc(F)cc2F)cc2cnn(Cc3ccc(F)cc3)c12 ; O=C(NC1(C23CC(C(=O)O)(C2)C3)CC1)c1cc(-c2ccc(F)c(F)c2)cc2cnn(Cc3ccc(F)cc3)c12 ; CC(C)(NC(=O)c1cc(-c2ccccc2)cc2cnn(Cc3ccc(F)cc3)c12)C12CC(C(=O)O)(C1)C2]

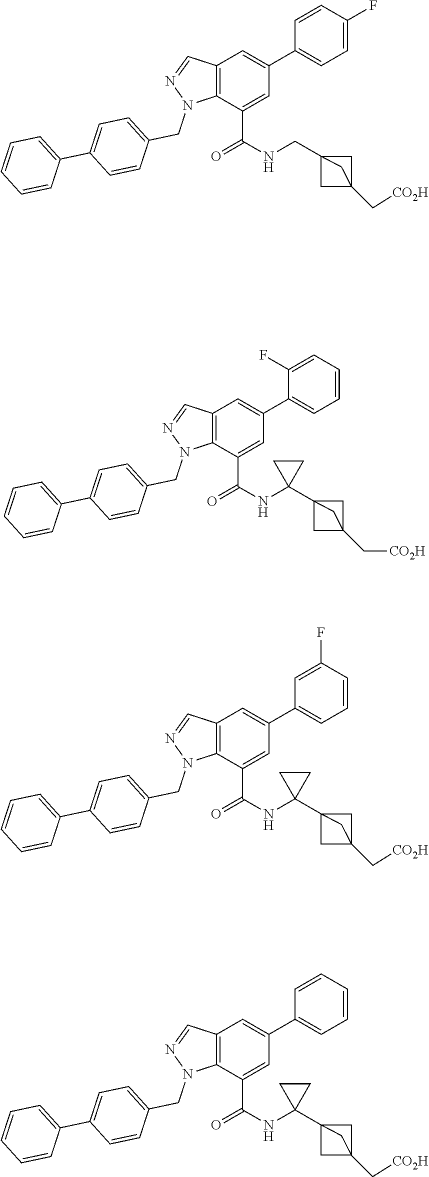 [SMILES: O=C(O)CC12CC(CNC(=O)c3cc(-c4ccc(F)cc4)cc4cnn(Cc5ccc(-c6ccccc6)cc5)c34)(C1)C2 ; O=C(O)CC12CC(C3(NC(=O)c4cc(-c5ccccc5F)cc5cnn(Cc6ccc(-c7ccccc7)cc6)c45)CC3)(C1)C2 ; O=C(O)CC12CC(C3(NC(=O)c4cc(-c5cccc(F)c5)cc5cnn(Cc6ccc(-c7ccccc7)cc6)c45)CC3)(C1)C2 ; O=C(O)CC12CC(C3(NC(=O)c4cc(-c5ccccc5)cc5cnn(Cc6ccc(-c7ccccc7)cc6)c45)CC3)(C1)C2]

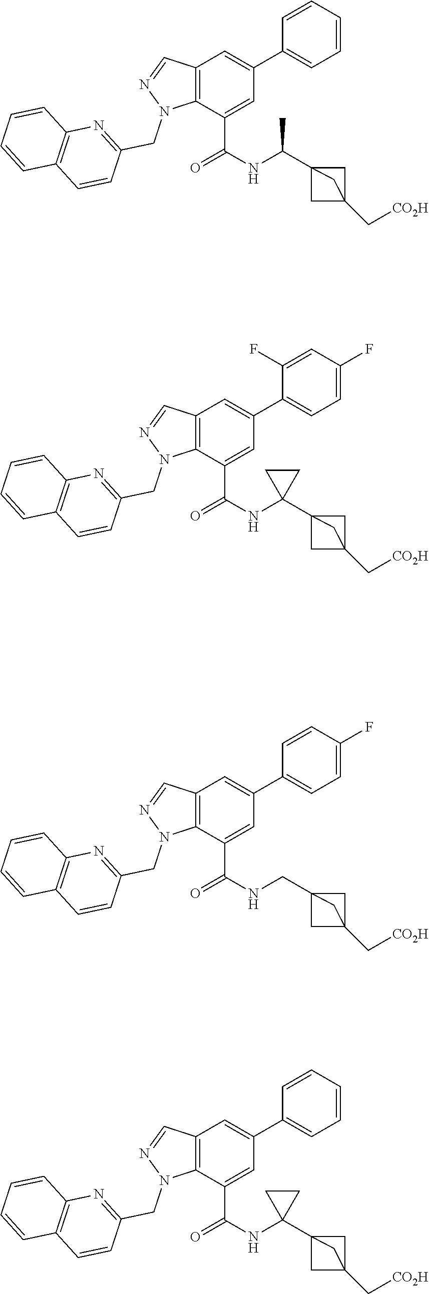 [SMILES: C[C@H](NC(=O)c1cc(-c2ccccc2)cc2cnn(Cc3ccc4ccccc4n3)c12)C12CC(CC(=O)O)(C1)C2 ; O=C(O)CC12CC(C3(NC(=O)c4cc(-c5ccc(F)cc5F)cc5cnn(Cc6ccc7ccccc7n6)c45)CC3)(C1)C2 ; O=C(O)CC12CC(CNC(=O)c3cc(-c4ccc(F)cc4)cc4cnn(Cc5ccc6ccccc6n5)c34)(C1)C2 ; O=C(O)CC12CC(C3(NC(=O)c4cc(-c5ccccc5)cc5cnn(Cc6ccc7ccccc7n6)c45)CC3)(C1)C2]

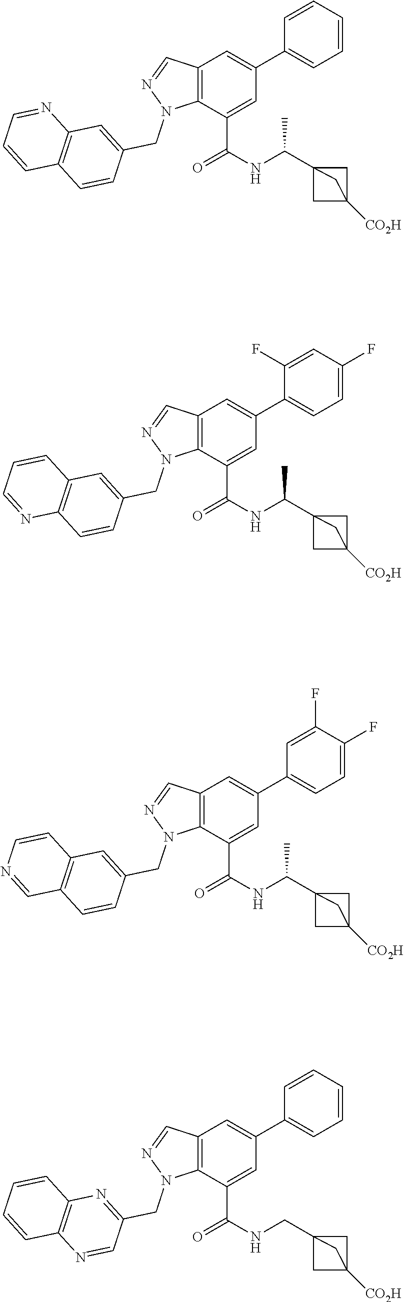 [SMILES: C[C@@H](NC(=O)c1cc(-c2ccccc2)cc2cnn(Cc3ccc4cccnc4c3)c12)C12CC(C(=O)O)(C1)C2 ; C[C@H](NC(=O)c1cc(-c2ccc(F)cc2F)cc2cnn(Cc3ccc4ncccc4c3)c12)C12CC(C(=O)O)(C1)C2 ; C[C@@H](NC(=O)c1cc(-c2ccc(F)c(F)c2)cc2cnn(Cc3ccc4cnccc4c3)c12)C12CC(C(=O)O)(C1)C2 ; O=C(NCC12CC(C(=O)O)(C1)C2)c1cc(-c2ccccc2)cc2cnn(Cc3cnc4ccccc4n3)c12]

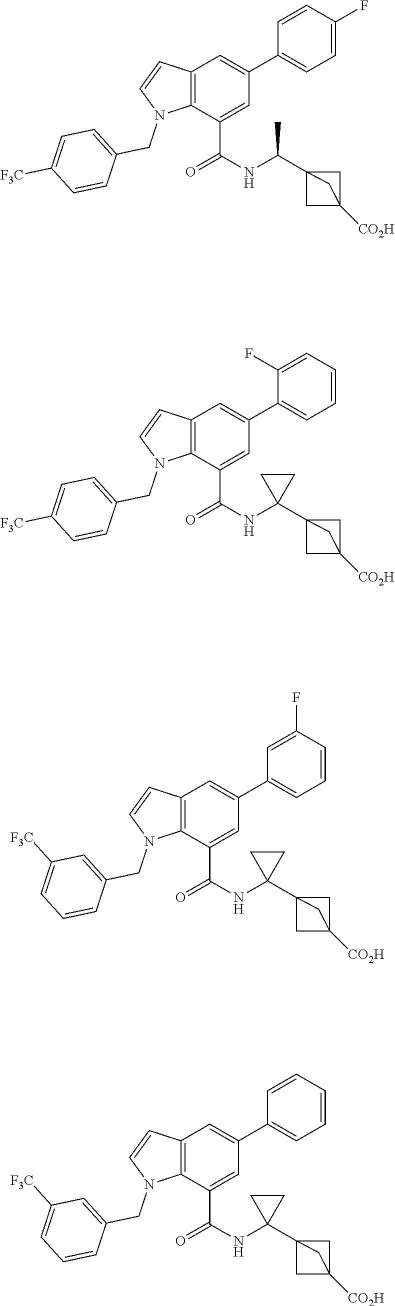 [SMILES: C[C@H](NC(=O)c1cc(-c2ccc(F)cc2)cc2ccn(Cc3ccc(C(F)(F)F)cc3)c12)C12CC(C(=O)O)(C1)C2 ; O=C(NC1(C23CC(C(=O)O)(C2)C3)CC1)c1cc(-c2ccccc2F)cc2ccn(Cc3ccc(C(F)(F)F)cc3)c12 ; O=C(NC1(C23CC(C(=O)O)(C2)C3)CC1)c1cc(-c2cccc(F)c2)cc2ccn(Cc3cccc(C(F)(F)F)c3)c12 ; O=C(NC1(C23CC(C(=O)O)(C2)C3)CC1)c1cc(-c2ccccc2)cc2ccn(Cc3cccc(C(F)(F)F)c3)c12]

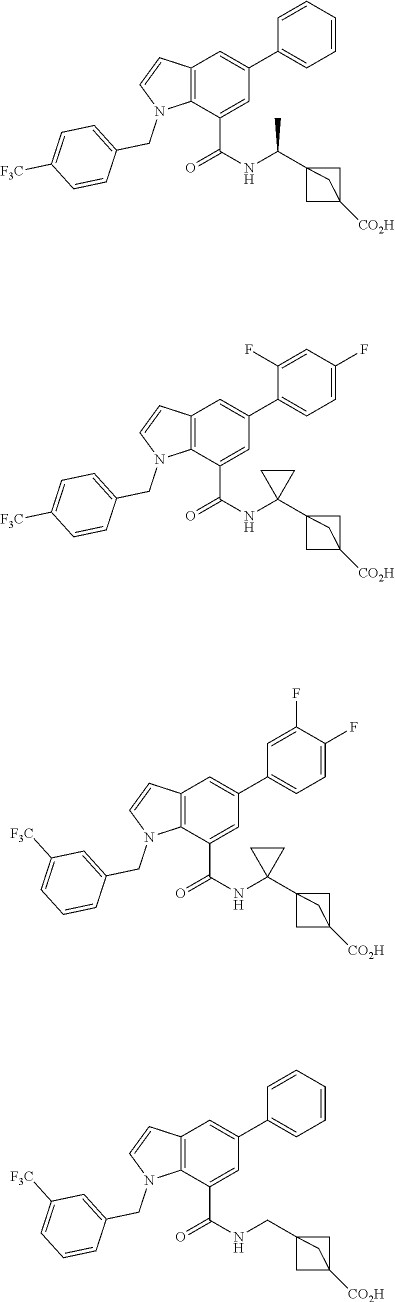 [SMILES: C[C@H](NC(=O)c1cc(-c2ccccc2)cc2ccn(Cc3ccc(C(F)(F)F)cc3)c12)C12CC(C(=O)O)(C1)C2 ; O=C(NC1(C23CC(C(=O)O)(C2)C3)CC1)c1cc(-c2ccc(F)cc2F)cc2ccn(Cc3ccc(C(F)(F)F)cc3)c12 ; O=C(NC1(C23CC(C(=O)O)(C2)C3)CC1)c1cc(-c2ccc(F)c(F)c2)cc2ccn(Cc3cccc(C(F)(F)F)c3)c12 ; O=C(NCC12CC(C(=O)O)(C1)C2)c1cc(-c2ccccc2)cc2ccn(Cc3cccc(C(F)(F)F)c3)c12]

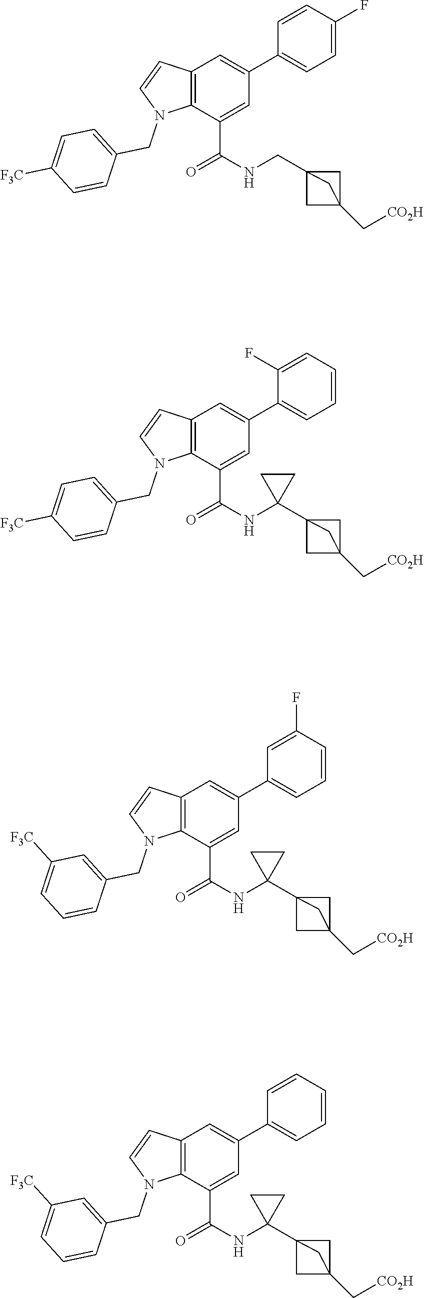 [SMILES: O=C(O)CC12CC(CNC(=O)c3cc(-c4ccc(F)cc4)cc4ccn(Cc5ccc(C(F)(F)F)cc5)c34)(C1)C2 ; O=C(O)CC12CC(C3(NC(=O)c4cc(-c5ccccc5F)cc5ccn(Cc6ccc(C(F)(F)F)cc6)c45)CC3)(C1)C2 ; O=C(O)CC12CC(C3(NC(=O)c4cc(-c5cccc(F)c5)cc5ccn(Cc6cccc(C(F)(F)F)c6)c45)CC3)(C1)C2 ; O=C(O)CC12CC(C3(NC(=O)c4cc(-c5ccccc5)cc5ccn(Cc6cccc(C(F)(F)F)c6)c45)CC3)(C1)C2]

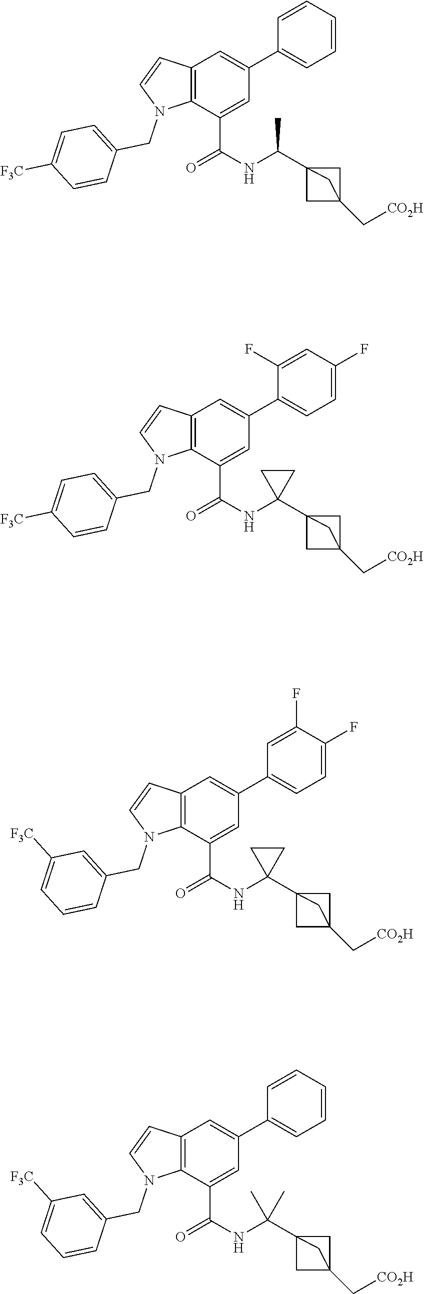 [SMILES: C[C@H](NC(=O)c1cc(-c2ccccc2)cc2ccn(Cc3ccc(C(F)(F)F)cc3)c12)C12CC(CC(=O)O)(C1)C2 ; O=C(O)CC12CC(C3(NC(=O)c4cc(-c5ccc(F)cc5F)cc5ccn(Cc6ccc(C(F)(F)F)cc6)c45)CC3)(C1)C2 ; O=C(O)CC12CC(C3(NC(=O)c4cc(-c5ccc(F)c(F)c5)cc5ccn(Cc6cccc(C(F)(F)F)c6)c45)CC3)(C1)C2 ; CC(C)(NC(=O)c1cc(-c2ccccc2)cc2ccn(Cc3cccc(C(F)(F)F)c3)c12)C12CC(CC(=O)O)(C1)C2]

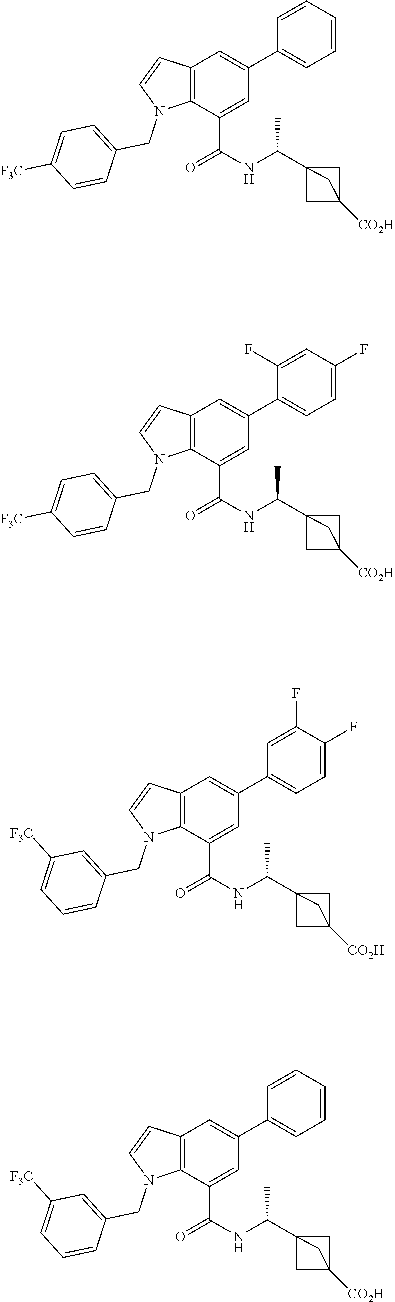 [SMILES: C[C@@H](NC(=O)c1cc(-c2ccccc2)cc2ccn(Cc3ccc(C(F)(F)F)cc3)c12)C12CC(C(=O)O)(C1)C2 ; C[C@H](NC(=O)c1cc(-c2ccc(F)cc2F)cc2ccn(Cc3ccc(C(F)(F)F)cc3)c12)C12CC(C(=O)O)(C1)C2 ; C[C@@H](NC(=O)c1cc(-c2ccc(F)c(F)c2)cc2ccn(Cc3cccc(C(F)(F)F)c3)c12)C12CC(C(=O)O)(C1)C2 ; C[C@@H](NC(=O)c1cc(-c2ccccc2)cc2ccn(Cc3cccc(C(F)(F)F)c3)c12)C12CC(C(=O)O)(C1)C2]

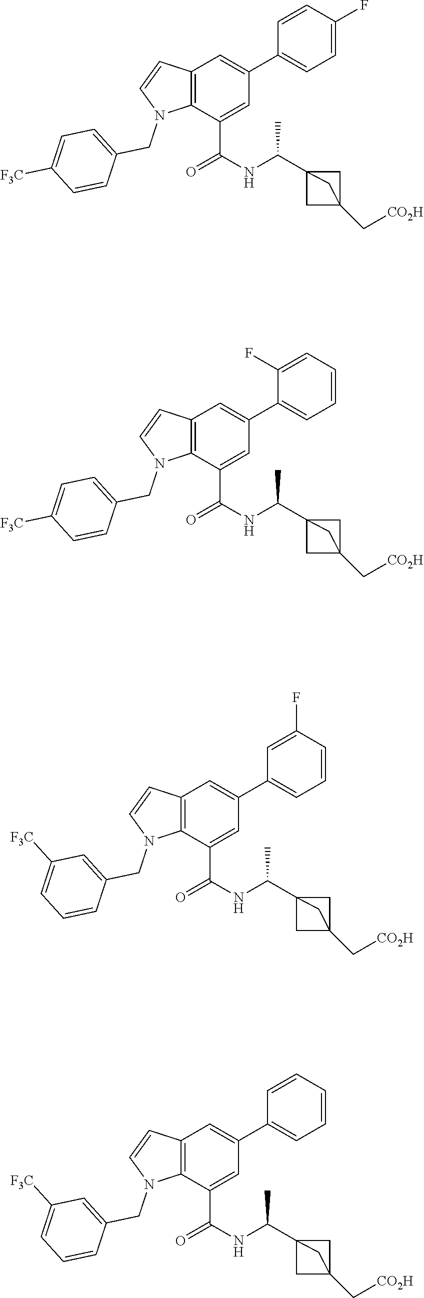 [SMILES: C[C@@H](NC(=O)c1cc(-c2ccc(F)cc2)cc2ccn(Cc3ccc(C(F)(F)F)cc3)c12)C12CC(CC(=O)O)(C1)C2 ; C[C@H](NC(=O)c1cc(-c2ccccc2F)cc2ccn(Cc3ccc(C(F)(F)F)cc3)c12)C12CC(CC(=O)O)(C1)C2 ; C[C@@H](NC(=O)c1cc(-c2cccc(F)c2)cc2ccn(Cc3cccc(C(F)(F)F)c3)c12)C12CC(CC(=O)O)(C1)C2 ; C[C@H](NC(=O)c1cc(-c2ccccc2)cc2ccn(Cc3cccc(C(F)(F)F)c3)c12)C12CC(CC(=O)O)(C1)C2]

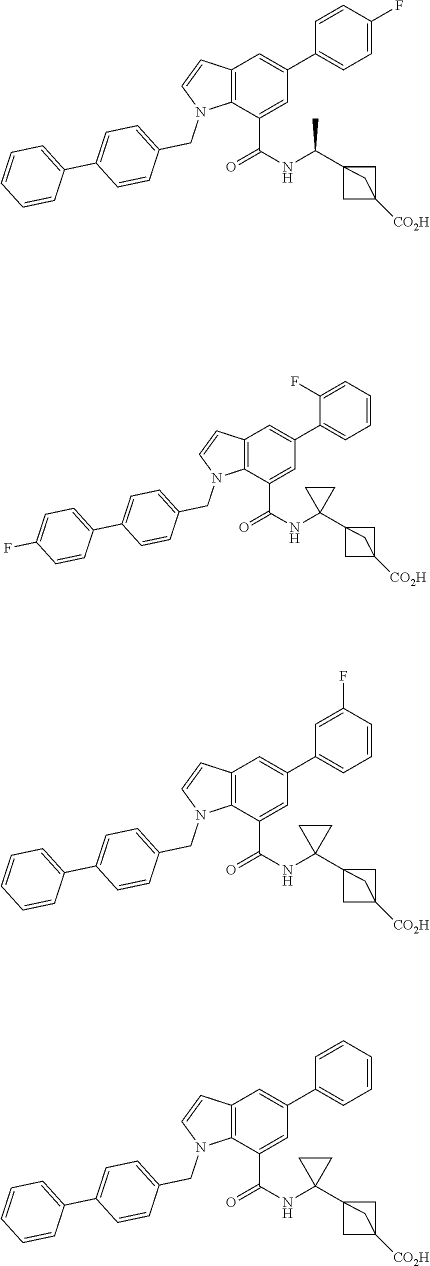 [SMILES: C[C@H](NC(=O)c1cc(-c2ccc(F)cc2)cc2ccn(Cc3ccc(-c4ccccc4)cc3)c12)C12CC(C(=O)O)(C1)C2 ; O=C(NC1(C23CC(C(=O)O)(C2)C3)CC1)c1cc(-c2ccccc2F)cc2ccn(Cc3ccc(-c4ccc(F)cc4)cc3)c12 ; O=C(NC1(C23CC(C(=O)O)(C2)C3)CC1)c1cc(-c2cccc(F)c2)cc2ccn(Cc3ccc(-c4ccccc4)cc3)c12 ; O=C(NC1(C23CC(C(=O)O)(C2)C3)CC1)c1cc(-c2ccccc2)cc2ccn(Cc3ccc(-c4ccccc4)cc3)c12]

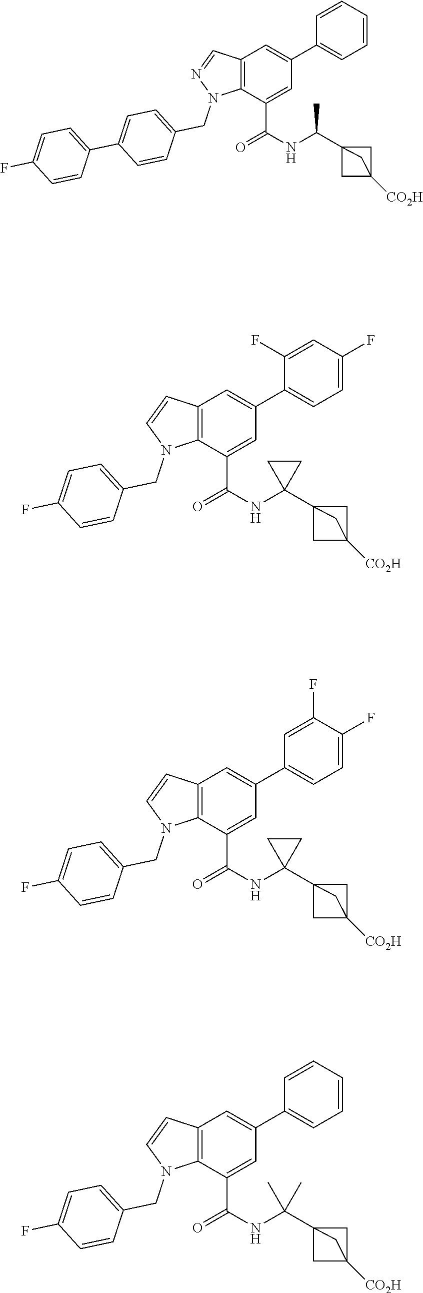 [SMILES: C[C@H](NC(=O)c1cc(-c2ccccc2)cc2cnn(Cc3ccc(-c4ccc(F)cc4)cc3)c12)C12CC(C(=O)O)(C1)C2 ; O=C(NC1(C23CC(C(=O)O)(C2)C3)CC1)c1cc(-c2ccc(F)cc2F)cc2ccn(Cc3ccc(F)cc3)c12 ; O=C(NC1(C23CC(C(=O)O)(C2)C3)CC1)c1cc(-c2ccc(F)c(F)c2)cc2ccn(Cc3ccc(F)cc3)c12 ; CC(C)(NC(=O)c1cc(-c2ccccc2)cc2ccn(Cc3ccc(F)cc3)c12)C12CC(C(=O)O)(C1)C2]

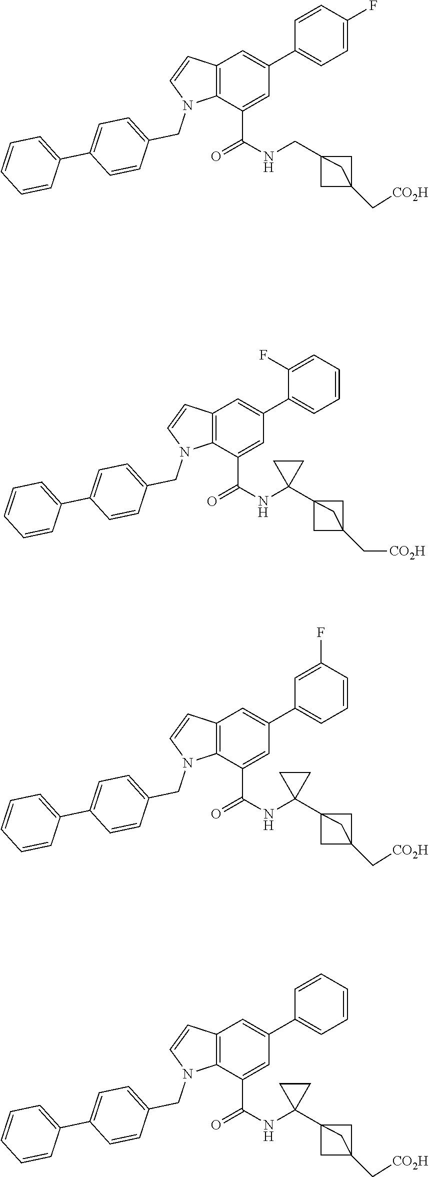 [SMILES: O=C(O)CC12CC(CNC(=O)c3cc(-c4ccc(F)cc4)cc4ccn(Cc5ccc(-c6ccccc6)cc5)c34)(C1)C2 ; O=C(O)CC12CC(C3(NC(=O)c4cc(-c5ccccc5F)cc5ccn(Cc6ccc(-c7ccccc7)cc6)c45)CC3)(C1)C2 ; O=C(O)CC12CC(C3(NC(=O)c4cc(-c5cccc(F)c5)cc5ccn(Cc6ccc(-c7ccccc7)cc6)c45)CC3)(C1)C2 ; O=C(O)CC12CC(C3(NC(=O)c4cc(-c5ccccc5)cc5ccn(Cc6ccc(-c7ccccc7)cc6)c45)CC3)(C1)C2]

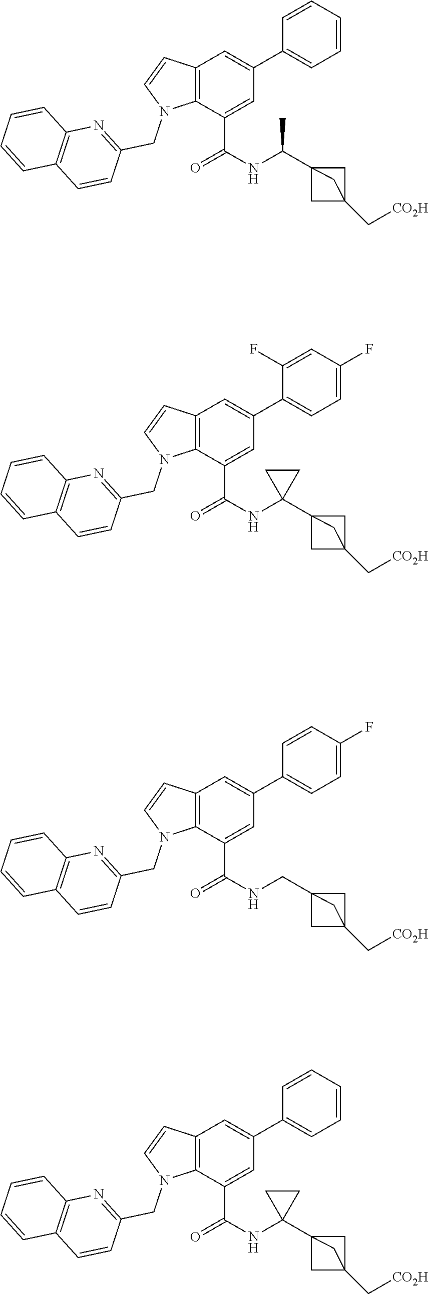 [SMILES: C[C@H](NC(=O)c1cc(-c2ccccc2)cc2ccn(Cc3ccc4ccccc4n3)c12)C12CC(CC(=O)O)(C1)C2 ; O=C(O)CC12CC(C3(NC(=O)c4cc(-c5ccc(F)cc5F)cc5ccn(Cc6ccc7ccccc7n6)c45)CC3)(C1)C2 ; O=C(O)CC12CC(CNC(=O)c3cc(-c4ccc(F)cc4)cc4ccn(Cc5ccc6ccccc6n5)c34)(C1)C2 ; O=C(O)CC12CC(C3(NC(=O)c4cc(-c5ccccc5)cc5ccn(Cc6ccc7ccccc7n6)c45)CC3)(C1)C2]

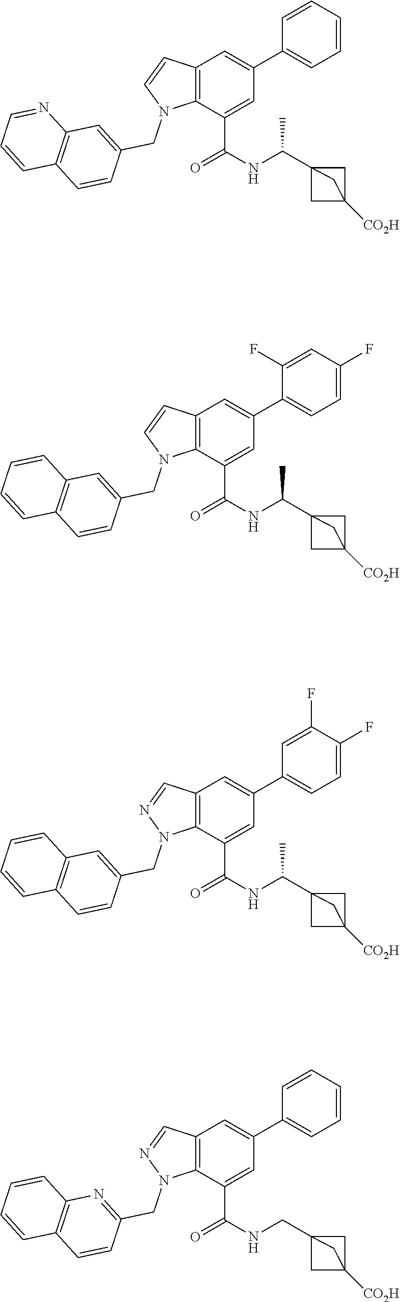 [SMILES: C[C@@H](NC(=O)c1cc(-c2ccccc2)cc2ccn(Cc3ccc4cccnc4c3)c12)C12CC(C(=O)O)(C1)C2 ; C[C@H](NC(=O)c1cc(-c2ccc(F)cc2F)cc2ccn(Cc3ccc4ccccc4c3)c12)C12CC(C(=O)O)(C1)C2 ; C[C@@H](NC(=O)c1cc(-c2ccc(F)c(F)c2)cc2cnn(Cc3ccc4ccccc4c3)c12)C12CC(C(=O)O)(C1)C2 ; O=C(NCC12CC(C(=O)O)(C1)C2)c1cc(-c2ccccc2)cc2cnn(Cc3ccc4ccccc4n3)c12]

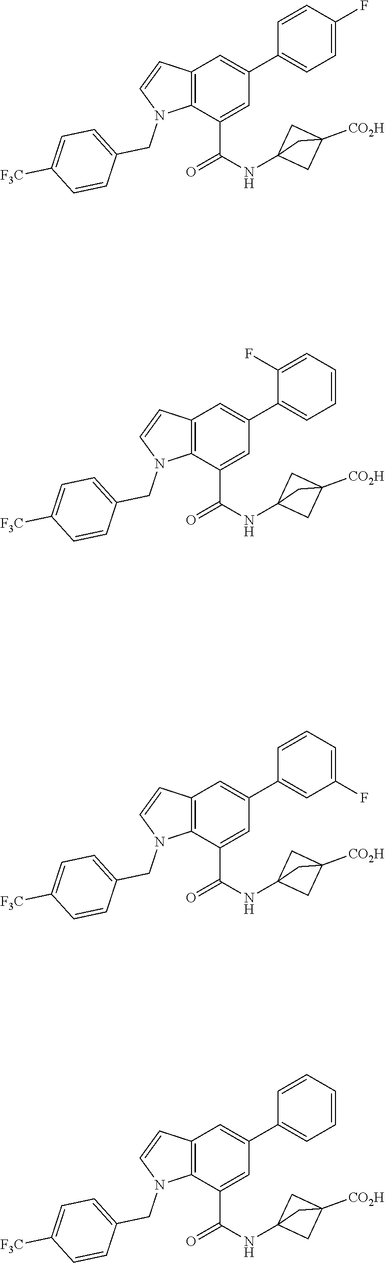 [SMILES: O=C(NC12CC(C(=O)O)(C1)C2)c1cc(-c2ccc(F)cc2)cc2ccn(Cc3ccc(C(F)(F)F)cc3)c12 ; O=C(NC12CC(C(=O)O)(C1)C2)c1cc(-c2ccccc2F)cc2ccn(Cc3ccc(C(F)(F)F)cc3)c12 ; O=C(NC12CC(C(=O)O)(C1)C2)c1cc(-c2cccc(F)c2)cc2ccn(Cc3ccc(C(F)(F)F)cc3)c12 ; O=C(NC12CC(C(=O)O)(C1)C2)c1cc(-c2ccccc2)cc2ccn(Cc3ccc(C(F)(F)F)cc3)c12]

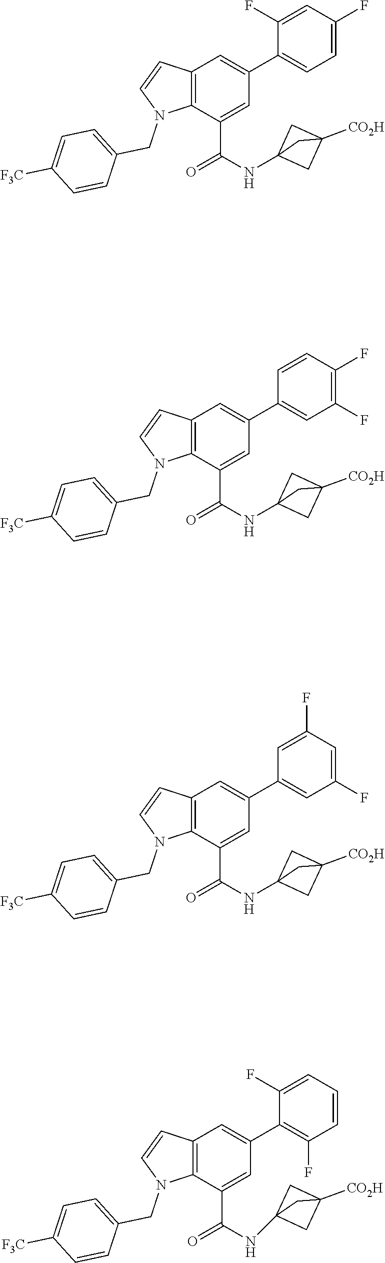 [SMILES: O=C(NC12CC(C(=O)O)(C1)C2)c1cc(-c2ccc(F)cc2F)cc2ccn(Cc3ccc(C(F)(F)F)cc3)c12 ; O=C(NC12CC(C(=O)O)(C1)C2)c1cc(-c2ccc(F)c(F)c2)cc2ccn(Cc3ccc(C(F)(F)F)cc3)c12 ; O=C(NC12CC(C(=O)O)(C1)C2)c1cc(-c2cc(F)cc(F)c2)cc2ccn(Cc3ccc(C(F)(F)F)cc3)c12 ; O=C(NC12CC(C(=O)O)(C1)C2)c1cc(-c2c(F)cccc2F)cc2ccn(Cc3ccc(C(F)(F)F)cc3)c12]

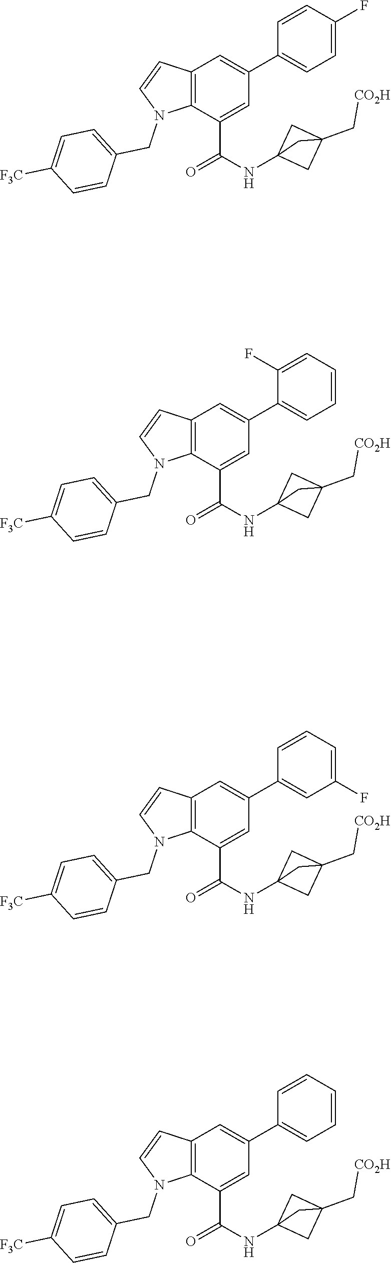 [SMILES: O=C(O)CC12CC(NC(=O)c3cc(-c4ccc(F)cc4)cc4ccn(Cc5ccc(C(F)(F)F)cc5)c34)(C1)C2 ; O=C(O)CC12CC(NC(=O)c3cc(-c4ccccc4F)cc4ccn(Cc5ccc(C(F)(F)F)cc5)c34)(C1)C2 ; O=C(O)CC12CC(NC(=O)c3cc(-c4cccc(F)c4)cc4ccn(Cc5ccc(C(F)(F)F)cc5)c34)(C1)C2 ; O=C(O)CC12CC(NC(=O)c3cc(-c4ccccc4)cc4ccn(Cc5ccc(C(F)(F)F)cc5)c34)(C1)C2]

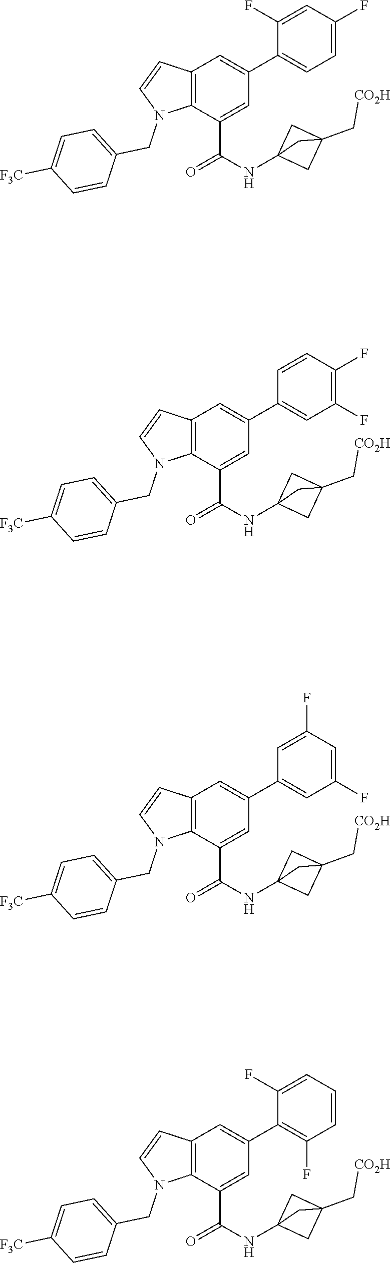 [SMILES: O=C(O)CC12CC(NC(=O)c3cc(-c4ccc(F)cc4F)cc4ccn(Cc5ccc(C(F)(F)F)cc5)c34)(C1)C2 ; O=C(O)CC12CC(NC(=O)c3cc(-c4ccc(F)c(F)c4)cc4ccn(Cc5ccc(C(F)(F)F)cc5)c34)(C1)C2 ; O=C(O)CC12CC(NC(=O)c3cc(-c4cc(F)cc(F)c4)cc4ccn(Cc5ccc(C(F)(F)F)cc5)c34)(C1)C2 ; O=C(O)CC12CC(NC(=O)c3cc(-c4c(F)cccc4F)cc4ccn(Cc5ccc(C(F)(F)F)cc5)c34)(C1)C2]

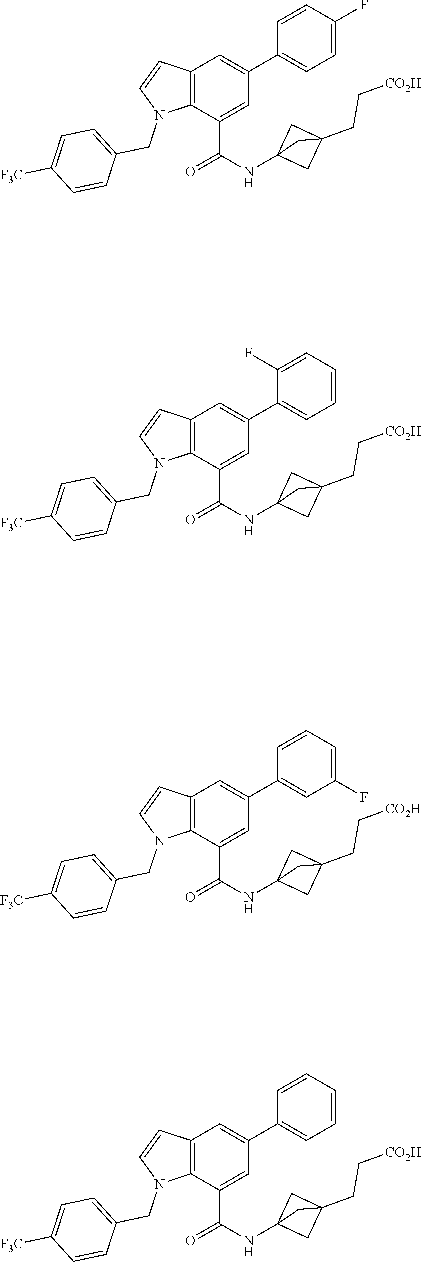 [SMILES: O=C(O)CCC12CC(NC(=O)c3cc(-c4ccc(F)cc4)cc4ccn(Cc5ccc(C(F)(F)F)cc5)c34)(C1)C2 ; O=C(O)CCC12CC(NC(=O)c3cc(-c4ccccc4F)cc4ccn(Cc5ccc(C(F)(F)F)cc5)c34)(C1)C2 ; O=C(O)CCC12CC(NC(=O)c3cc(-c4cccc(F)c4)cc4ccn(Cc5ccc(C(F)(F)F)cc5)c34)(C1)C2 ; O=C(O)CCC12CC(NC(=O)c3cc(-c4ccccc4)cc4ccn(Cc5ccc(C(F)(F)F)cc5)c34)(C1)C2]

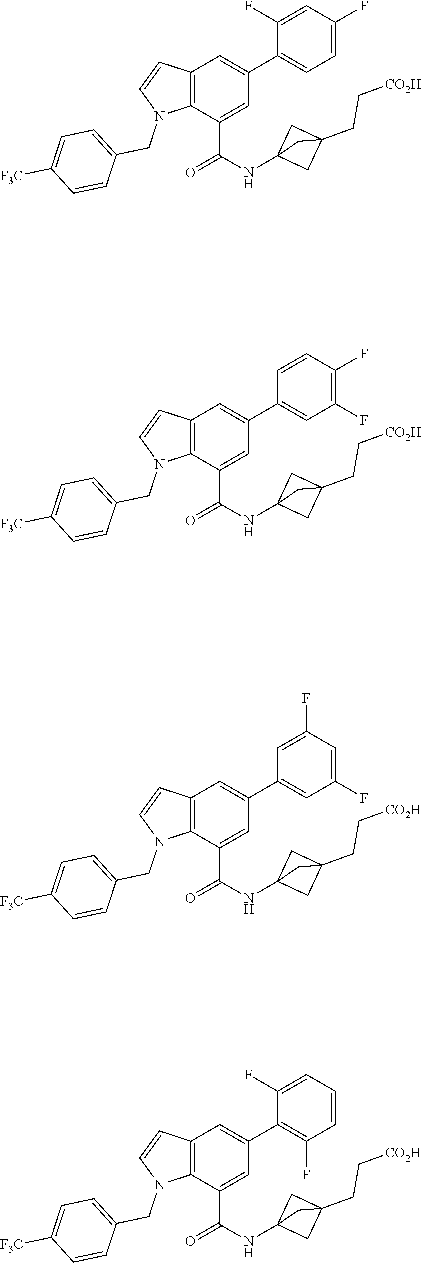 [SMILES: O=C(O)CCC12CC(NC(=O)c3cc(-c4ccc(F)cc4F)cc4ccn(Cc5ccc(C(F)(F)F)cc5)c34)(C1)C2 ; O=C(O)CCC12CC(NC(=O)c3cc(-c4ccc(F)c(F)c4)cc4ccn(Cc5ccc(C(F)(F)F)cc5)c34)(C1)C2 ; O=C(O)CCC12CC(NC(=O)c3cc(-c4cc(F)cc(F)c4)cc4ccn(Cc5ccc(C(F)(F)F)cc5)c34)(C1)C2 ; O=C(O)CCC12CC(NC(=O)c3cc(-c4c(F)cccc4F)cc4ccn(Cc5ccc(C(F)(F)F)cc5)c34)(C1)C2]

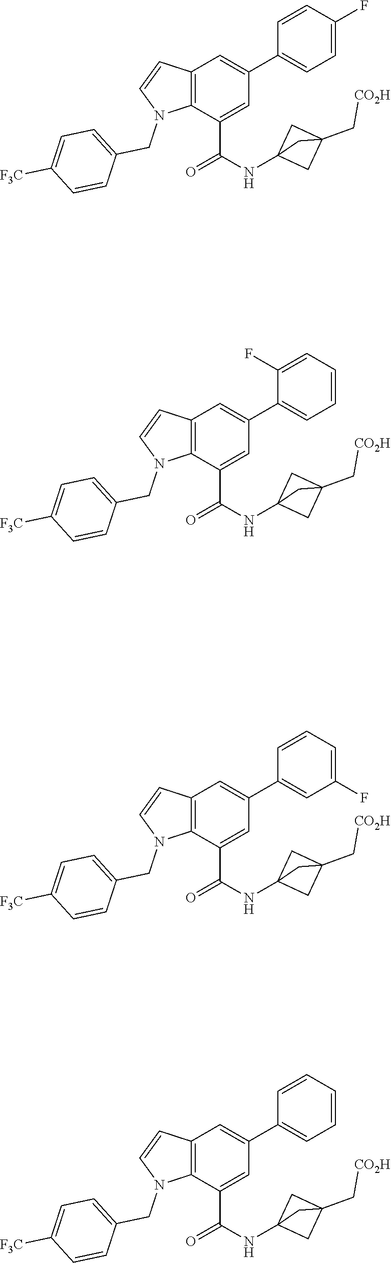 [SMILES: O=C(O)CC12CC(NC(=O)c3cc(-c4ccc(F)cc4)cc4ccn(Cc5ccc(C(F)(F)F)cc5)c34)(C1)C2 ; O=C(O)CC12CC(NC(=O)c3cc(-c4ccccc4F)cc4ccn(Cc5ccc(C(F)(F)F)cc5)c34)(C1)C2 ; O=C(O)CC12CC(NC(=O)c3cc(-c4cccc(F)c4)cc4ccn(Cc5ccc(C(F)(F)F)cc5)c34)(C1)C2 ; O=C(O)CC12CC(NC(=O)c3cc(-c4ccccc4)cc4ccn(Cc5ccc(C(F)(F)F)cc5)c34)(C1)C2]

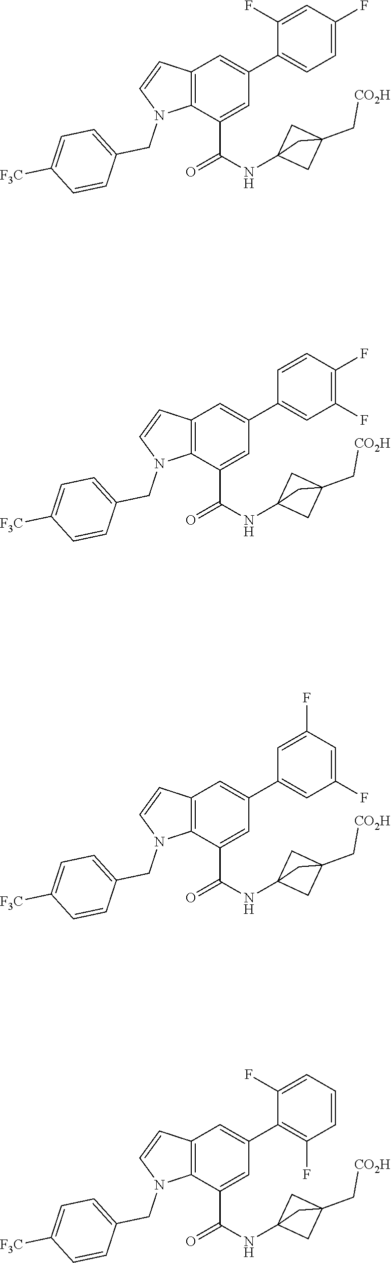 [SMILES: O=C(O)CC12CC(NC(=O)c3cc(-c4ccc(F)cc4F)cc4ccn(Cc5ccc(C(F)(F)F)cc5)c34)(C1)C2 ; O=C(O)CC12CC(NC(=O)c3cc(-c4ccc(F)c(F)c4)cc4ccn(Cc5ccc(C(F)(F)F)cc5)c34)(C1)C2 ; O=C(O)CC12CC(NC(=O)c3cc(-c4cc(F)cc(F)c4)cc4ccn(Cc5ccc(C(F)(F)F)cc5)c34)(C1)C2 ; O=C(O)CC12CC(NC(=O)c3cc(-c4c(F)cccc4F)cc4ccn(Cc5ccc(C(F)(F)F)cc5)c34)(C1)C2]

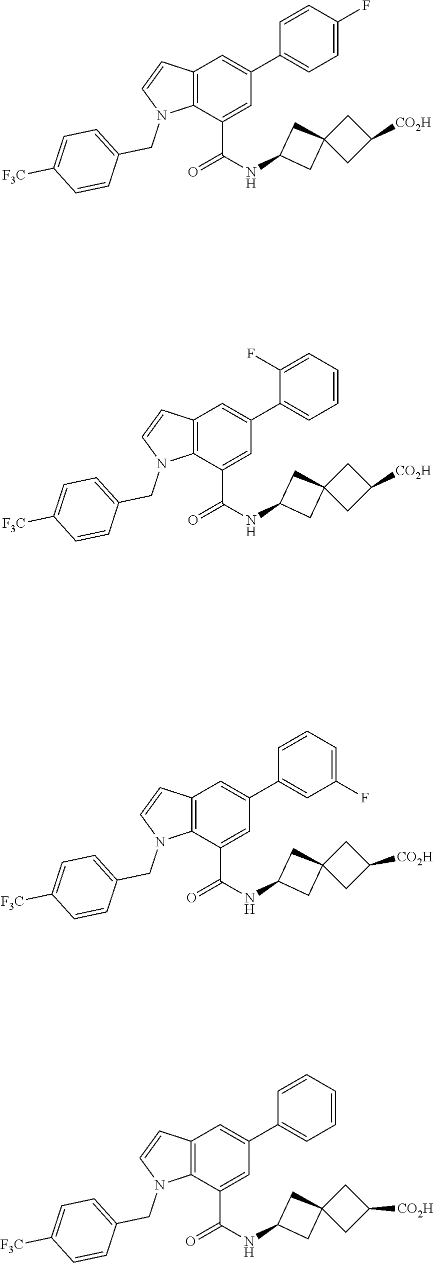

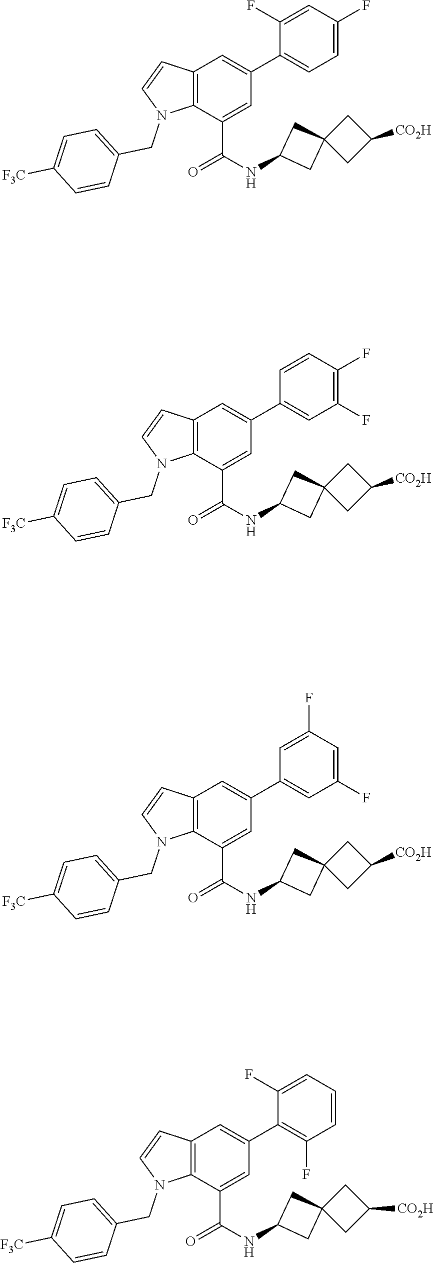

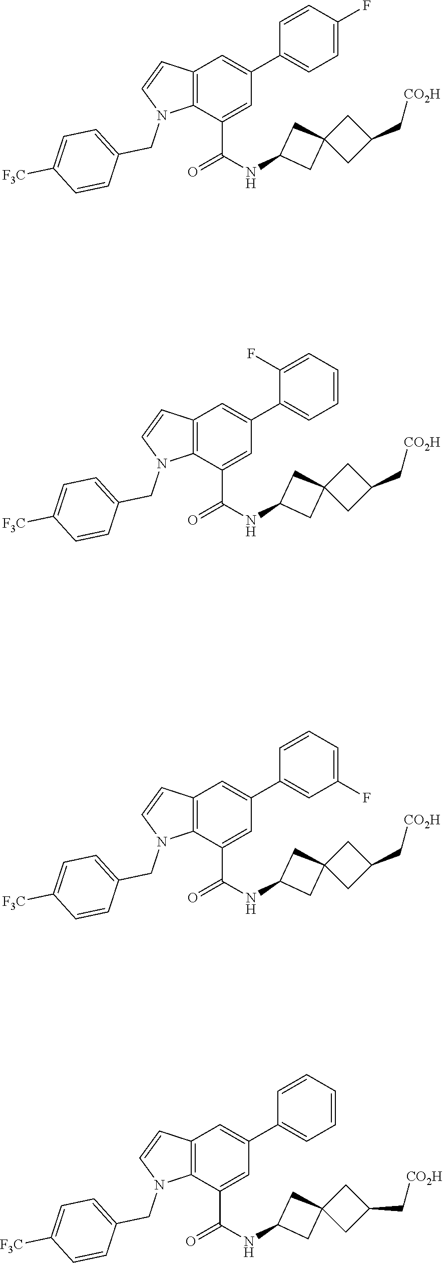

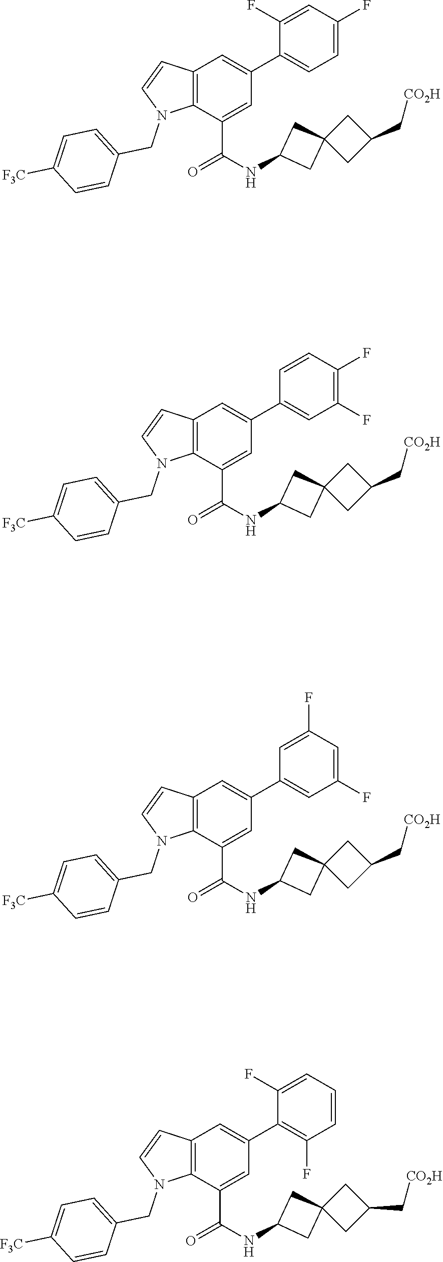

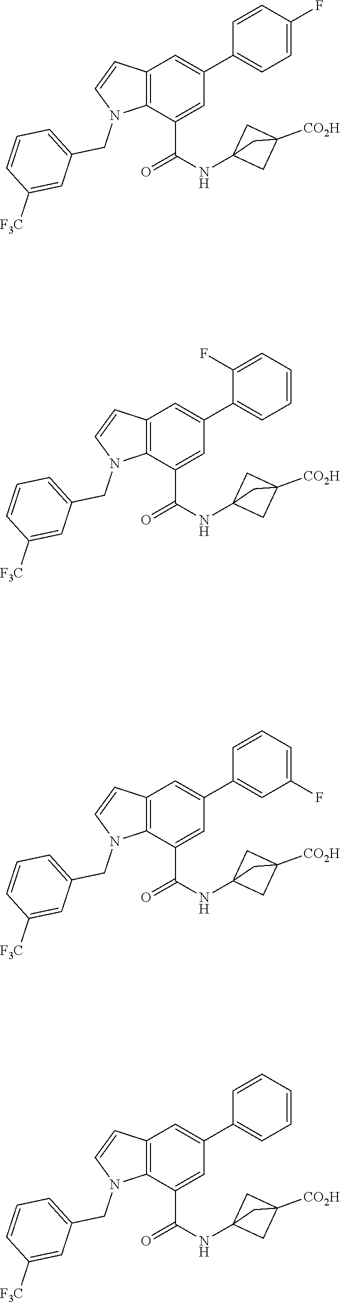 [SMILES: O=C(NC12CC(C(=O)O)(C1)C2)c1cc(-c2ccc(F)cc2)cc2ccn(Cc3cccc(C(F)(F)F)c3)c12 ; O=C(NC12CC(C(=O)O)(C1)C2)c1cc(-c2ccccc2F)cc2ccn(Cc3cccc(C(F)(F)F)c3)c12 ; O=C(NC12CC(C(=O)O)(C1)C2)c1cc(-c2cccc(F)c2)cc2ccn(Cc3cccc(C(F)(F)F)c3)c12 ; O=C(NC12CC(C(=O)O)(C1)C2)c1cc(-c2ccccc2)cc2ccn(Cc3cccc(C(F)(F)F)c3)c12]

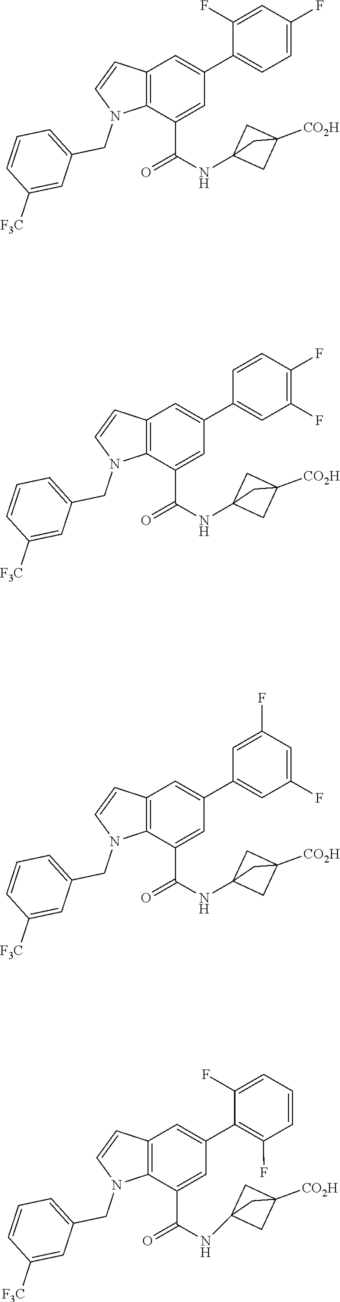 [SMILES: O=C(NC12CC(C(=O)O)(C1)C2)c1cc(-c2ccc(F)cc2F)cc2ccn(Cc3cccc(C(F)(F)F)c3)c12 ; O=C(NC12CC(C(=O)O)(C1)C2)c1cc(-c2ccc(F)c(F)c2)cc2ccn(Cc3cccc(C(F)(F)F)c3)c12 ; O=C(NC12CC(C(=O)O)(C1)C2)c1cc(-c2cc(F)cc(F)c2)cc2ccn(Cc3cccc(C(F)(F)F)c3)c12 ; O=C(NC12CC(C(=O)O)(C1)C2)c1cc(-c2c(F)cccc2F)cc2ccn(Cc3cccc(C(F)(F)F)c3)c12]

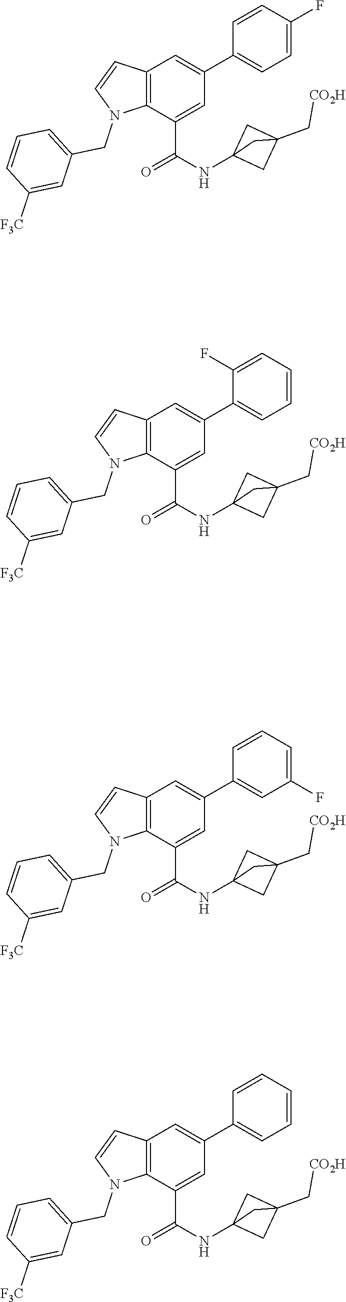 [SMILES: O=C(O)CC12CC(NC(=O)c3cc(-c4ccc(F)cc4)cc4ccn(Cc5cccc(C(F)(F)F)c5)c34)(C1)C2 ; O=C(O)CC12CC(NC(=O)c3cc(-c4ccccc4F)cc4ccn(Cc5cccc(C(F)(F)F)c5)c34)(C1)C2 ; O=C(O)CC12CC(NC(=O)c3cc(-c4cccc(F)c4)cc4ccn(Cc5cccc(C(F)(F)F)c5)c34)(C1)C2 ; O=C(O)CC12CC(NC(=O)c3cc(-c4ccccc4)cc4ccn(Cc5cccc(C(F)(F)F)c5)c34)(C1)C2]

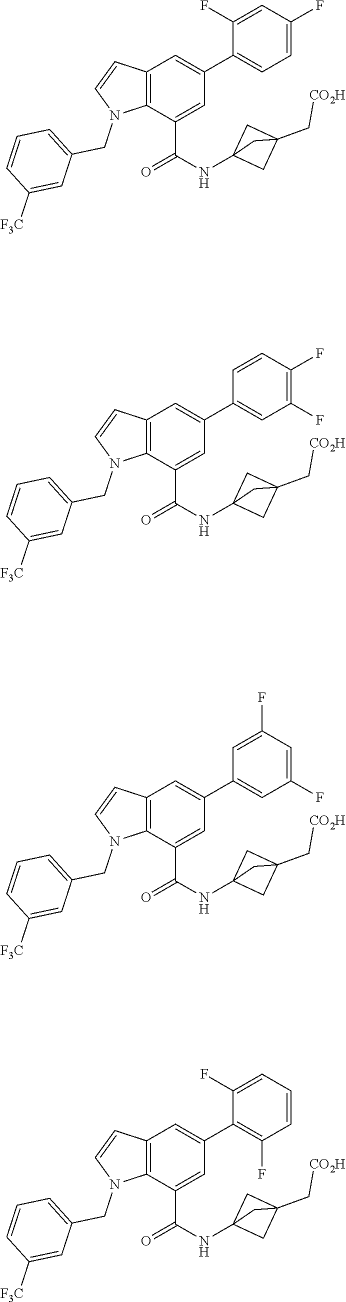 [SMILES: O=C(O)CC12CC(NC(=O)c3cc(-c4ccc(F)cc4F)cc4ccn(Cc5cccc(C(F)(F)F)c5)c34)(C1)C2 ; O=C(O)CC12CC(NC(=O)c3cc(-c4ccc(F)c(F)c4)cc4ccn(Cc5cccc(C(F)(F)F)c5)c34)(C1)C2 ; O=C(O)CC12CC(NC(=O)c3cc(-c4cc(F)cc(F)c4)cc4ccn(Cc5cccc(C(F)(F)F)c5)c34)(C1)C2 ; O=C(O)CC12CC(NC(=O)c3cc(-c4c(F)cccc4F)cc4ccn(Cc5cccc(C(F)(F)F)c5)c34)(C1)C2]

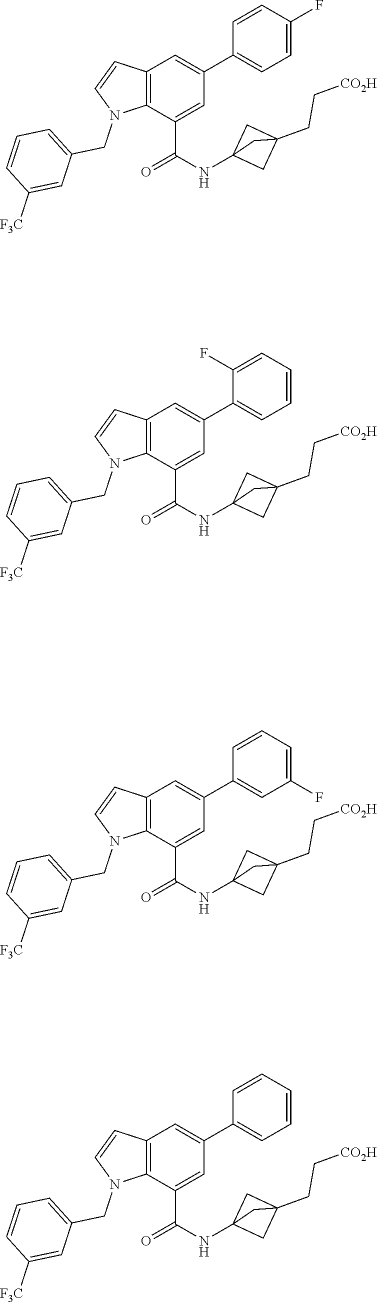 [SMILES: O=C(O)CCC12CC(NC(=O)c3cc(-c4ccc(F)cc4)cc4ccn(Cc5cccc(C(F)(F)F)c5)c34)(C1)C2 ; O=C(O)CCC12CC(NC(=O)c3cc(-c4ccccc4F)cc4ccn(Cc5cccc(C(F)(F)F)c5)c34)(C1)C2 ; O=C(O)CCC12CC(NC(=O)c3cc(-c4cccc(F)c4)cc4ccn(Cc5cccc(C(F)(F)F)c5)c34)(C1)C2 ; O=C(O)CCC12CC(NC(=O)c3cc(-c4ccccc4)cc4ccn(Cc5cccc(C(F)(F)F)c5)c34)(C1)C2]

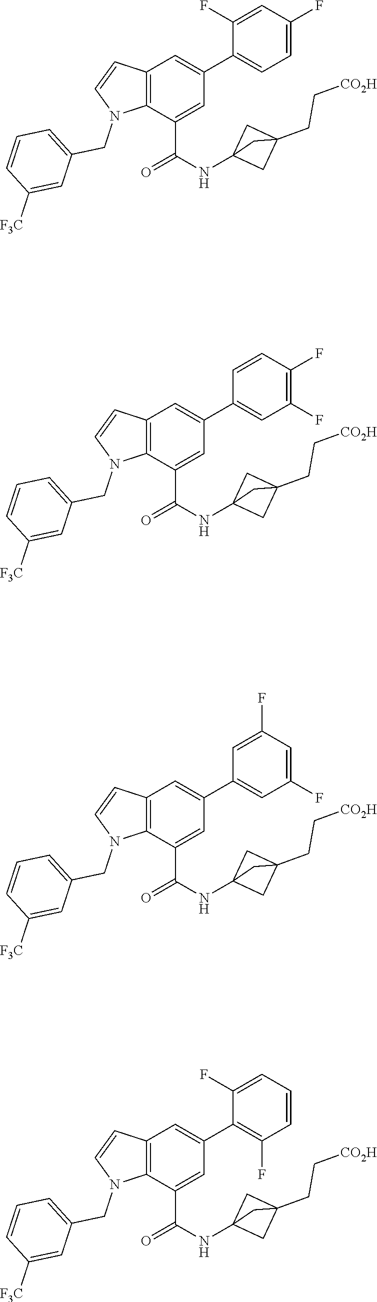 [SMILES: O=C(O)CCC12CC(NC(=O)c3cc(-c4ccc(F)cc4F)cc4ccn(Cc5cccc(C(F)(F)F)c5)c34)(C1)C2 ; O=C(O)CCC12CC(NC(=O)c3cc(-c4ccc(F)c(F)c4)cc4ccn(Cc5cccc(C(F)(F)F)c5)c34)(C1)C2 ; O=C(O)CCC12CC(NC(=O)c3cc(-c4cc(F)cc(F)c4)cc4ccn(Cc5cccc(C(F)(F)F)c5)c34)(C1)C2 ; O=C(O)CCC12CC(NC(=O)c3cc(-c4c(F)cccc4F)cc4ccn(Cc5cccc(C(F)(F)F)c5)c34)(C1)C2]

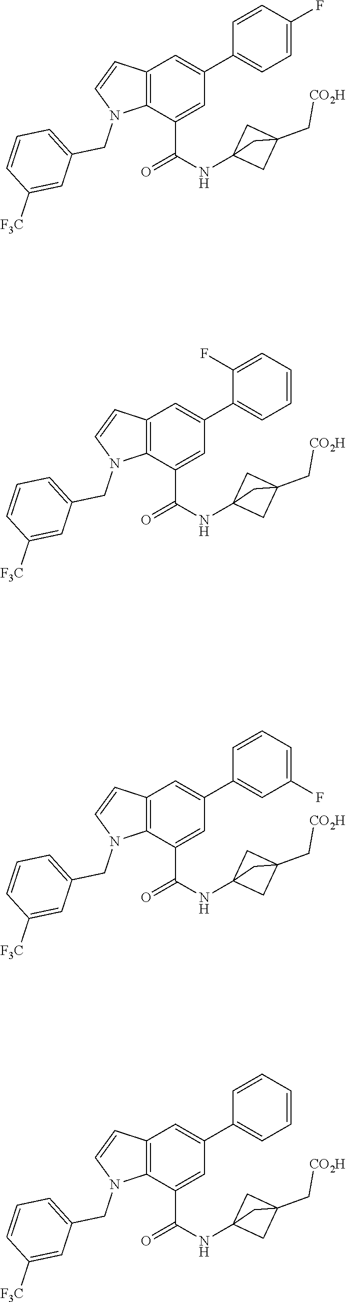 [SMILES: O=C(O)CC12CC(NC(=O)c3cc(-c4ccc(F)cc4)cc4ccn(Cc5cccc(C(F)(F)F)c5)c34)(C1)C2 ; O=C(O)CC12CC(NC(=O)c3cc(-c4ccccc4F)cc4ccn(Cc5cccc(C(F)(F)F)c5)c34)(C1)C2 ; O=C(O)CC12CC(NC(=O)c3cc(-c4cccc(F)c4)cc4ccn(Cc5cccc(C(F)(F)F)c5)c34)(C1)C2 ; O=C(O)CC12CC(NC(=O)c3cc(-c4ccccc4)cc4ccn(Cc5cccc(C(F)(F)F)c5)c34)(C1)C2]

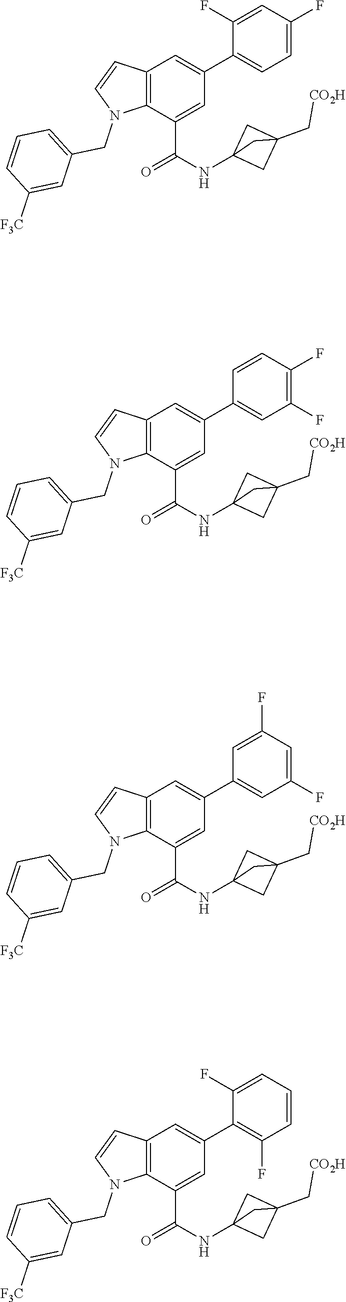 [SMILES: O=C(O)CC12CC(NC(=O)c3cc(-c4ccc(F)cc4F)cc4ccn(Cc5cccc(C(F)(F)F)c5)c34)(C1)C2 ; O=C(O)CC12CC(NC(=O)c3cc(-c4ccc(F)c(F)c4)cc4ccn(Cc5cccc(C(F)(F)F)c5)c34)(C1)C2 ; O=C(O)CC12CC(NC(=O)c3cc(-c4cc(F)cc(F)c4)cc4ccn(Cc5cccc(C(F)(F)F)c5)c34)(C1)C2 ; O=C(O)CC12CC(NC(=O)c3cc(-c4c(F)cccc4F)cc4ccn(Cc5cccc(C(F)(F)F)c5)c34)(C1)C2]

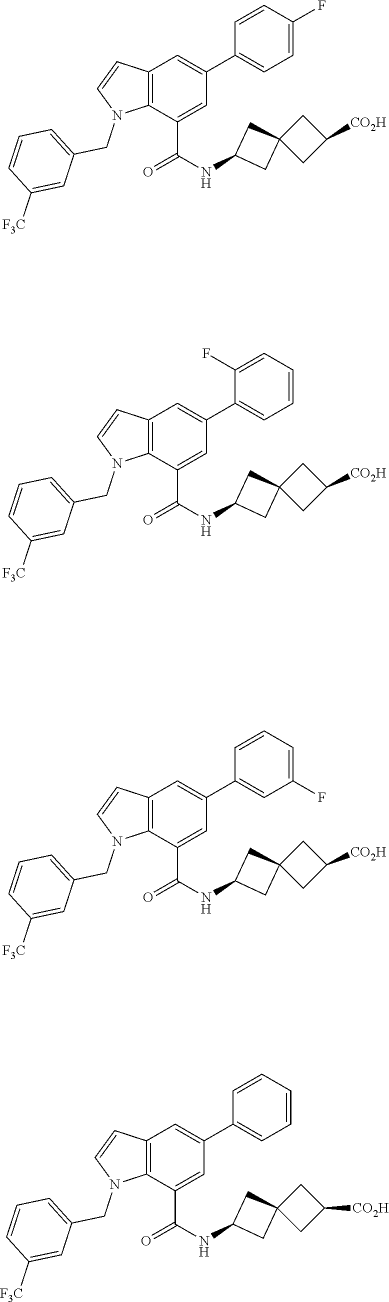

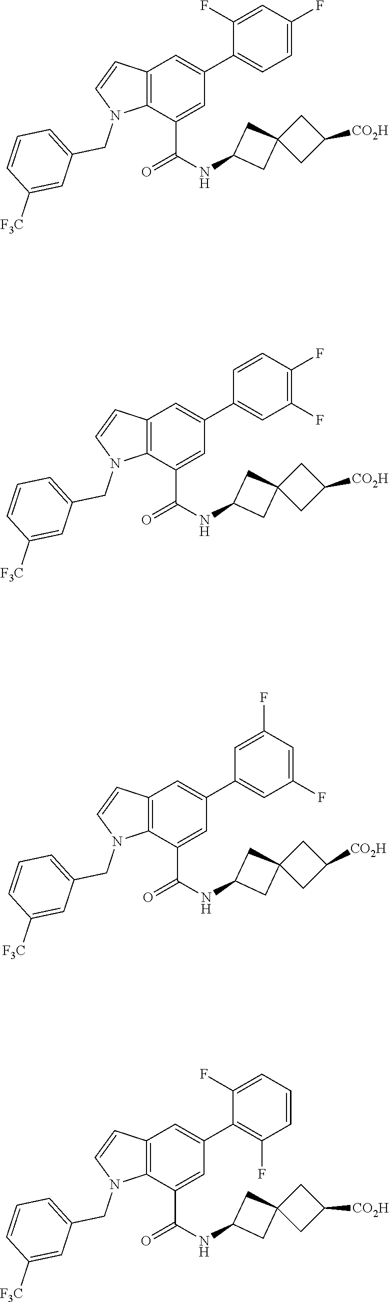

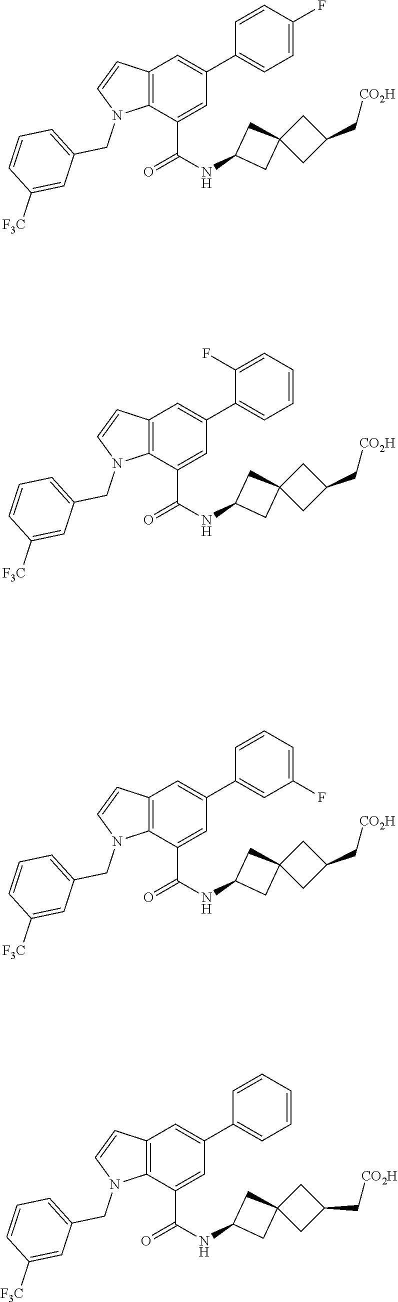

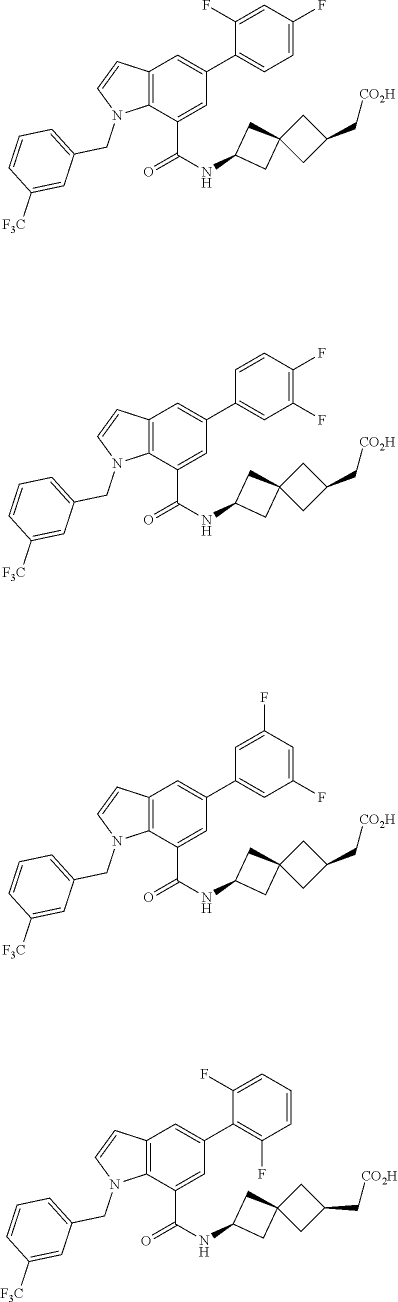

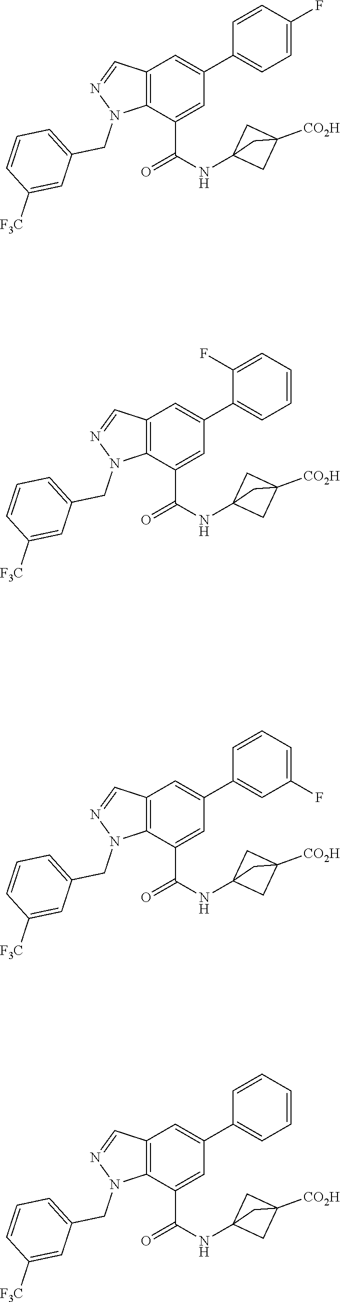 [SMILES: O=C(NC12CC(C(=O)O)(C1)C2)c1cc(-c2ccc(F)cc2)cc2cnn(Cc3cccc(C(F)(F)F)c3)c12 ; O=C(NC12CC(C(=O)O)(C1)C2)c1cc(-c2ccccc2F)cc2cnn(Cc3cccc(C(F)(F)F)c3)c12 ; O=C(NC12CC(C(=O)O)(C1)C2)c1cc(-c2cccc(F)c2)cc2cnn(Cc3cccc(C(F)(F)F)c3)c12 ; O=C(NC12CC(C(=O)O)(C1)C2)c1cc(-c2ccccc2)cc2cnn(Cc3cccc(C(F)(F)F)c3)c12]

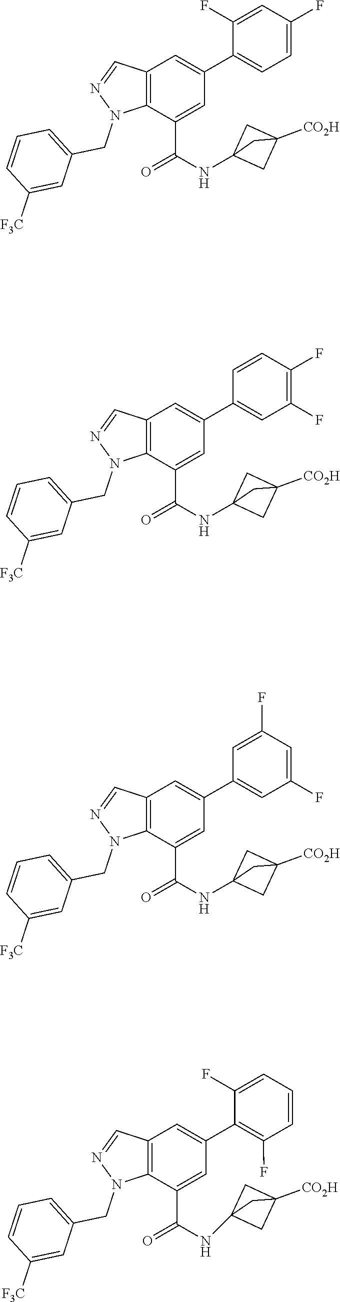 [SMILES: O=C(NC12CC(C(=O)O)(C1)C2)c1cc(-c2ccc(F)cc2F)cc2cnn(Cc3cccc(C(F)(F)F)c3)c12 ; O=C(NC12CC(C(=O)O)(C1)C2)c1cc(-c2ccc(F)c(F)c2)cc2cnn(Cc3cccc(C(F)(F)F)c3)c12 ; O=C(NC12CC(C(=O)O)(C1)C2)c1cc(-c2cc(F)cc(F)c2)cc2cnn(Cc3cccc(C(F)(F)F)c3)c12 ; O=C(NC12CC(C(=O)O)(C1)C2)c1cc(-c2c(F)cccc2F)cc2cnn(Cc3cccc(C(F)(F)F)c3)c12]

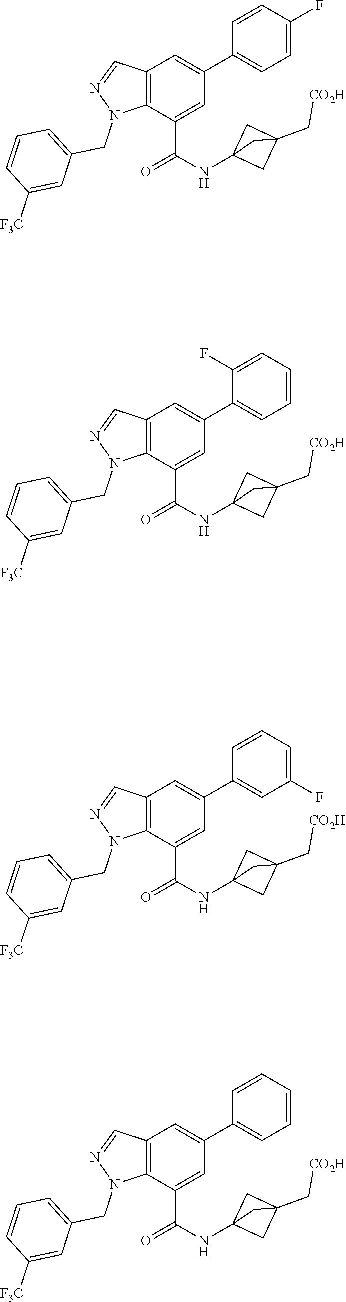 [SMILES: O=C(O)CC12CC(NC(=O)c3cc(-c4ccc(F)cc4)cc4cnn(Cc5cccc(C(F)(F)F)c5)c34)(C1)C2 ; O=C(O)CC12CC(NC(=O)c3cc(-c4ccccc4F)cc4cnn(Cc5cccc(C(F)(F)F)c5)c34)(C1)C2 ; O=C(O)CC12CC(NC(=O)c3cc(-c4cccc(F)c4)cc4cnn(Cc5cccc(C(F)(F)F)c5)c34)(C1)C2 ; O=C(O)CC12CC(NC(=O)c3cc(-c4ccccc4)cc4cnn(Cc5cccc(C(F)(F)F)c5)c34)(C1)C2]

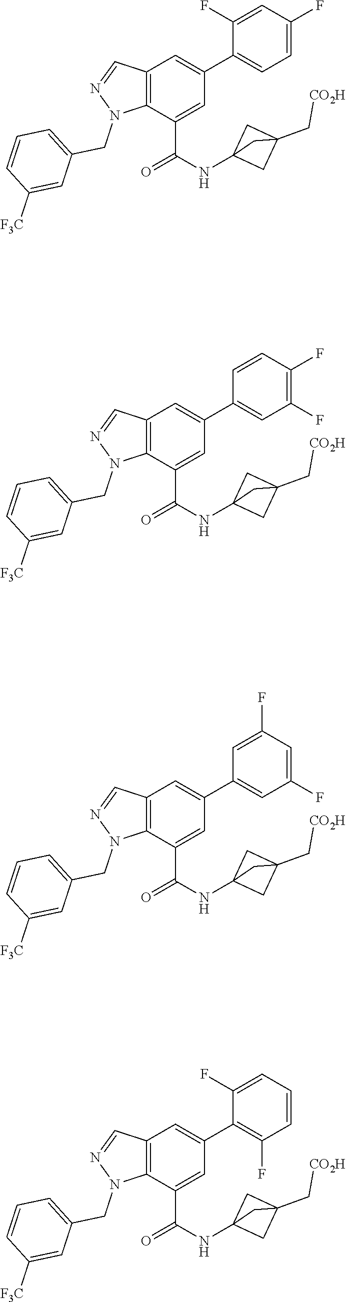 [SMILES: O=C(O)CC12CC(NC(=O)c3cc(-c4ccc(F)cc4F)cc4cnn(Cc5cccc(C(F)(F)F)c5)c34)(C1)C2 ; O=C(O)CC12CC(NC(=O)c3cc(-c4ccc(F)c(F)c4)cc4cnn(Cc5cccc(C(F)(F)F)c5)c34)(C1)C2 ; O=C(O)CC12CC(NC(=O)c3cc(-c4cc(F)cc(F)c4)cc4cnn(Cc5cccc(C(F)(F)F)c5)c34)(C1)C2 ; O=C(O)CC12CC(NC(=O)c3cc(-c4c(F)cccc4F)cc4cnn(Cc5cccc(C(F)(F)F)c5)c34)(C1)C2]

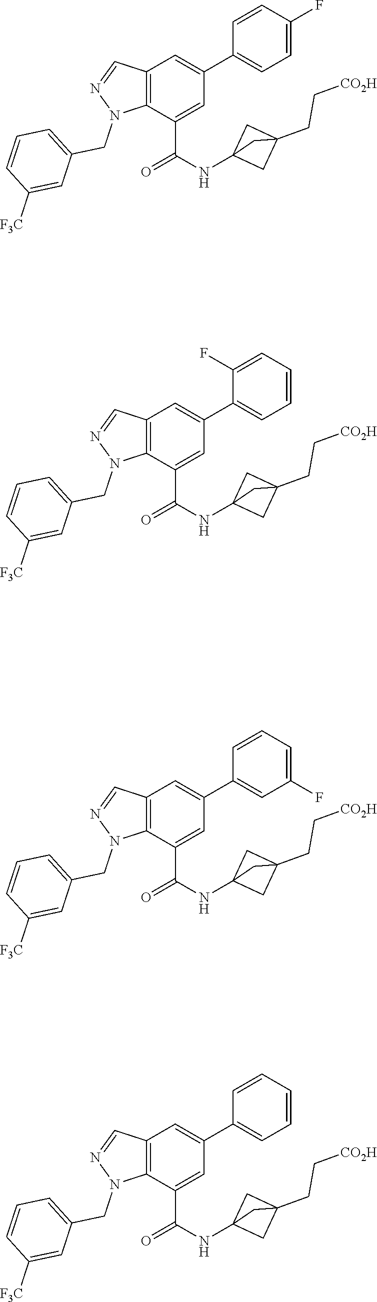 [SMILES: O=C(O)CCC12CC(NC(=O)c3cc(-c4ccc(F)cc4)cc4cnn(Cc5cccc(C(F)(F)F)c5)c34)(C1)C2 ; O=C(O)CCC12CC(NC(=O)c3cc(-c4ccccc4F)cc4cnn(Cc5cccc(C(F)(F)F)c5)c34)(C1)C2 ; O=C(O)CCC12CC(NC(=O)c3cc(-c4cccc(F)c4)cc4cnn(Cc5cccc(C(F)(F)F)c5)c34)(C1)C2 ; O=C(O)CCC12CC(NC(=O)c3cc(-c4ccccc4)cc4cnn(Cc5cccc(C(F)(F)F)c5)c34)(C1)C2]

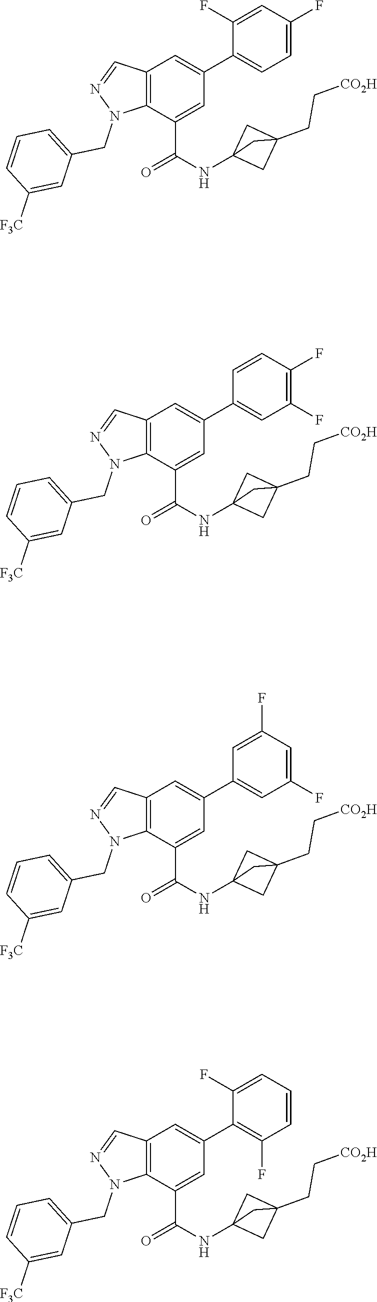 [SMILES: O=C(O)CCC12CC(NC(=O)c3cc(-c4ccc(F)cc4F)cc4cnn(Cc5cccc(C(F)(F)F)c5)c34)(C1)C2 ; O=C(O)CCC12CC(NC(=O)c3cc(-c4ccc(F)c(F)c4)cc4cnn(Cc5cccc(C(F)(F)F)c5)c34)(C1)C2 ; O=C(O)CCC12CC(NC(=O)c3cc(-c4cc(F)cc(F)c4)cc4cnn(Cc5cccc(C(F)(F)F)c5)c34)(C1)C2 ; O=C(O)CCC12CC(NC(=O)c3cc(-c4c(F)cccc4F)cc4cnn(Cc5cccc(C(F)(F)F)c5)c34)(C1)C2]

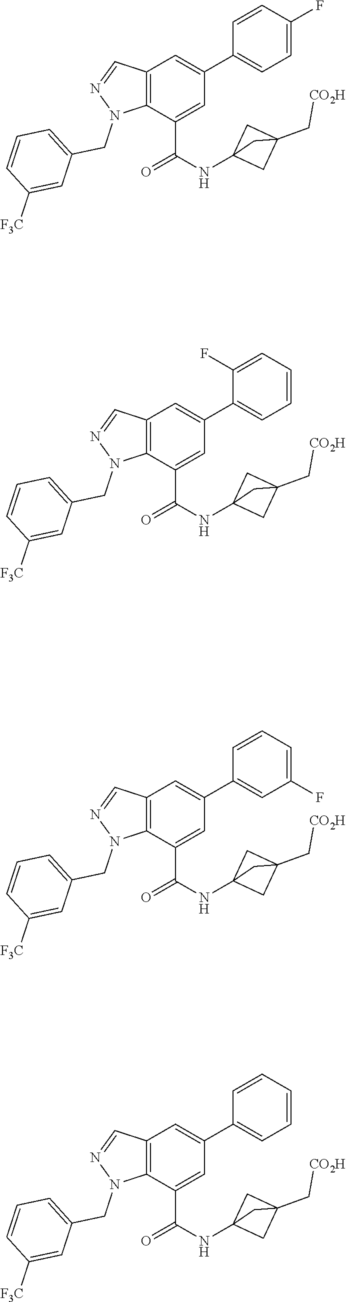 [SMILES: O=C(O)CC12CC(NC(=O)c3cc(-c4ccc(F)cc4)cc4cnn(Cc5cccc(C(F)(F)F)c5)c34)(C1)C2 ; O=C(O)CC12CC(NC(=O)c3cc(-c4ccccc4F)cc4cnn(Cc5cccc(C(F)(F)F)c5)c34)(C1)C2 ; O=C(O)CC12CC(NC(=O)c3cc(-c4cccc(F)c4)cc4cnn(Cc5cccc(C(F)(F)F)c5)c34)(C1)C2 ; O=C(O)CC12CC(NC(=O)c3cc(-c4ccccc4)cc4cnn(Cc5cccc(C(F)(F)F)c5)c34)(C1)C2]

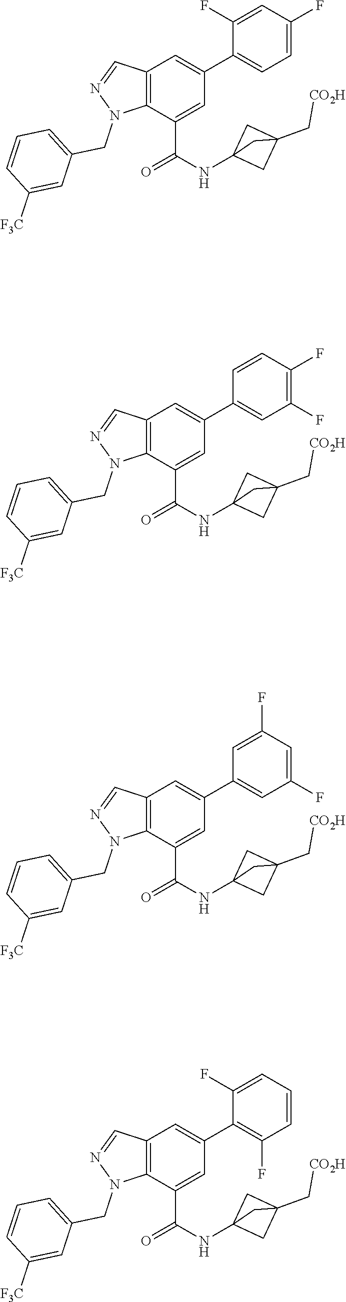 [SMILES: O=C(O)CC12CC(NC(=O)c3cc(-c4ccc(F)cc4F)cc4cnn(Cc5cccc(C(F)(F)F)c5)c34)(C1)C2 ; O=C(O)CC12CC(NC(=O)c3cc(-c4ccc(F)c(F)c4)cc4cnn(Cc5cccc(C(F)(F)F)c5)c34)(C1)C2 ; O=C(O)CC12CC(NC(=O)c3cc(-c4cc(F)cc(F)c4)cc4cnn(Cc5cccc(C(F)(F)F)c5)c34)(C1)C2 ; O=C(O)CC12CC(NC(=O)c3cc(-c4c(F)cccc4F)cc4cnn(Cc5cccc(C(F)(F)F)c5)c34)(C1)C2]

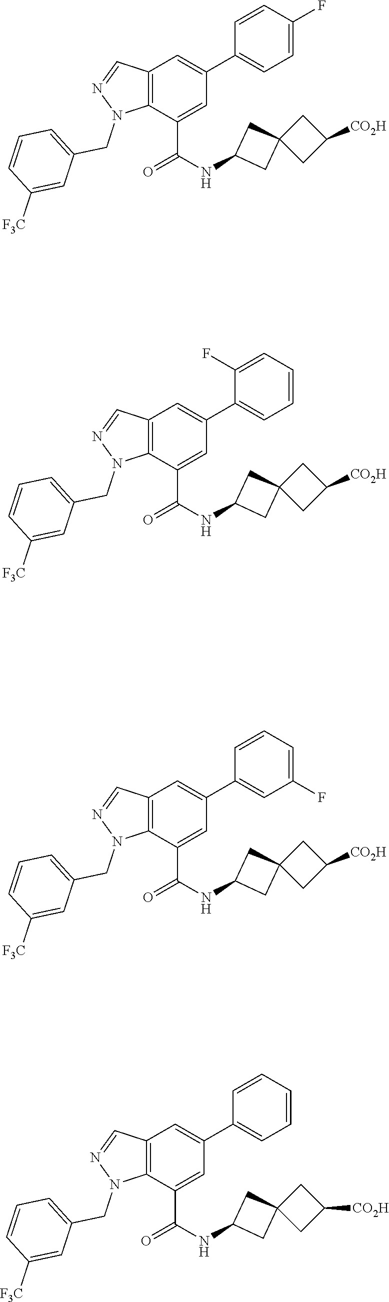

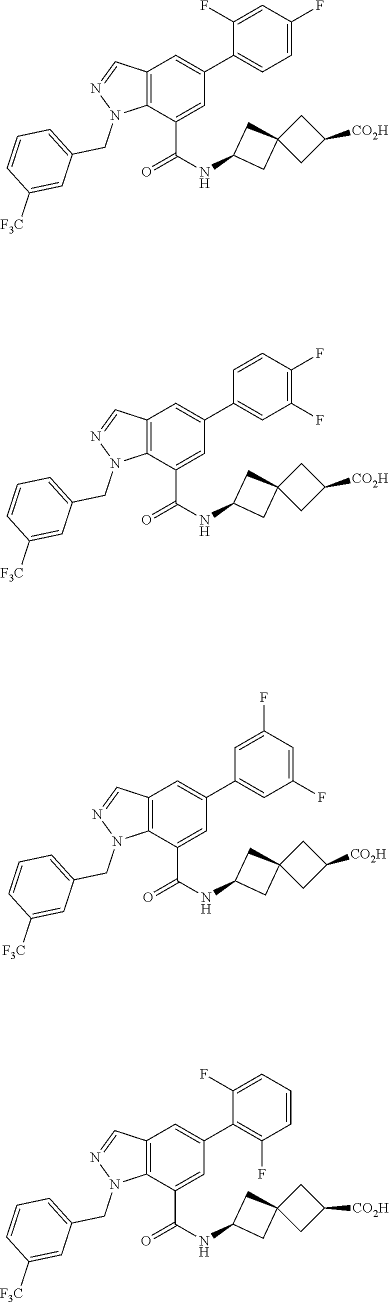

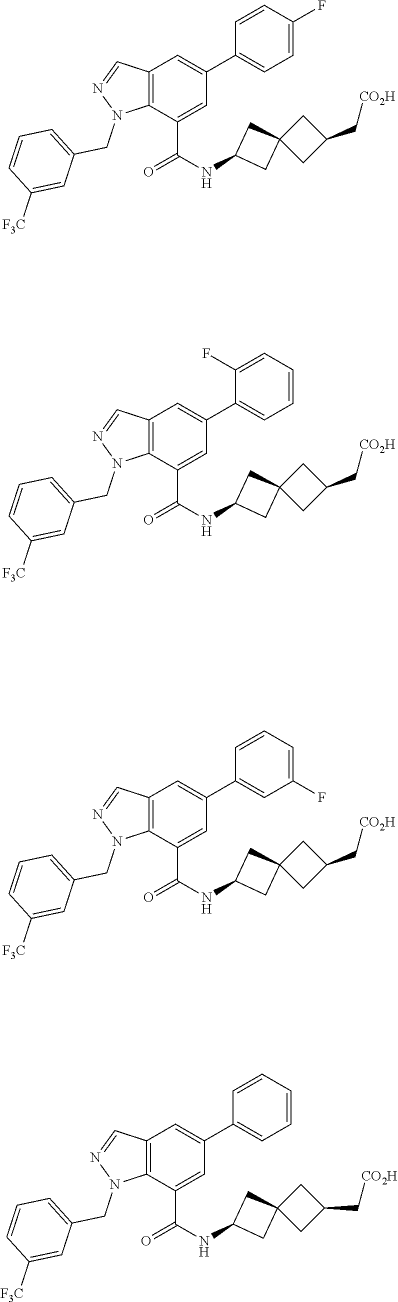

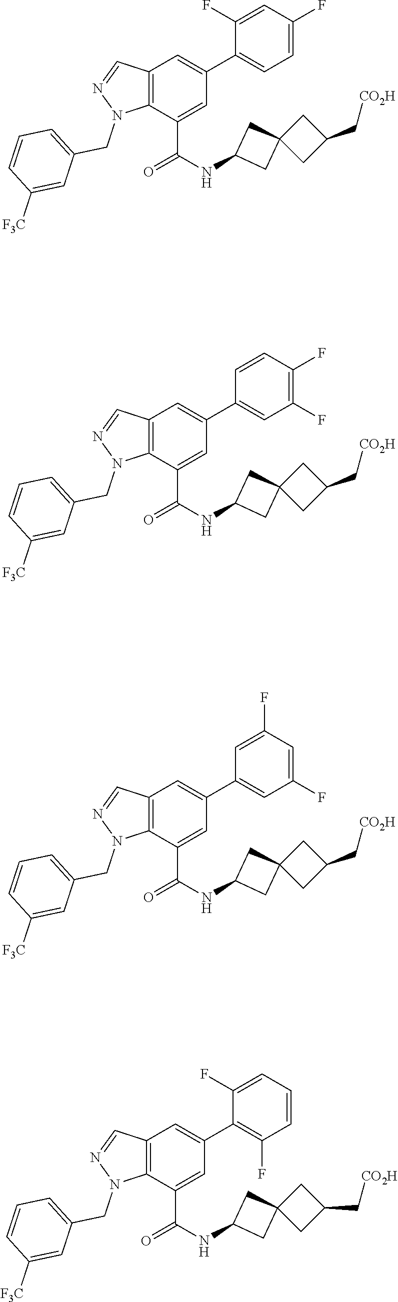

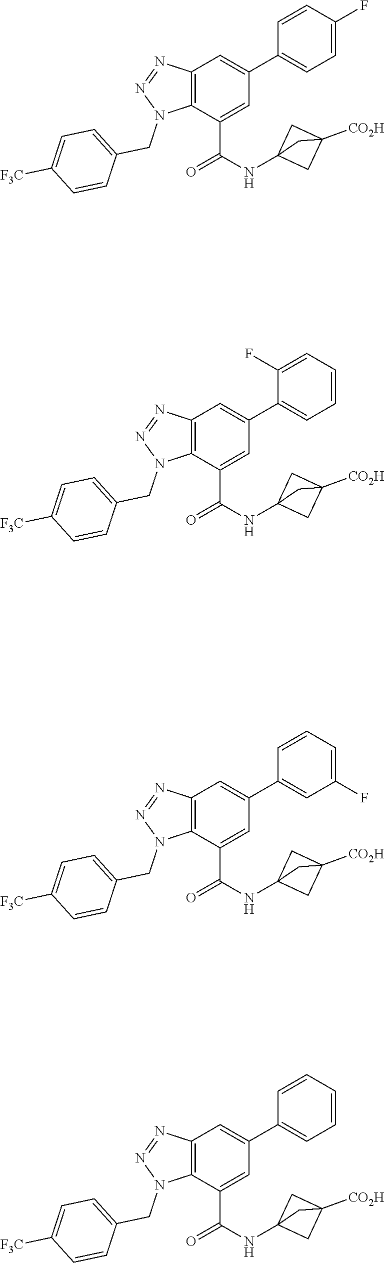 [SMILES: O=C(NC12CC(C(=O)O)(C1)C2)c1cc(-c2ccc(F)cc2)cc2nnn(Cc3ccc(C(F)(F)F)cc3)c12 ; O=C(NC12CC(C(=O)O)(C1)C2)c1cc(-c2ccccc2F)cc2nnn(Cc3ccc(C(F)(F)F)cc3)c12 ; O=C(NC12CC(C(=O)O)(C1)C2)c1cc(-c2cccc(F)c2)cc2nnn(Cc3ccc(C(F)(F)F)cc3)c12 ; O=C(NC12CC(C(=O)O)(C1)C2)c1cc(-c2ccccc2)cc2nnn(Cc3ccc(C(F)(F)F)cc3)c12]

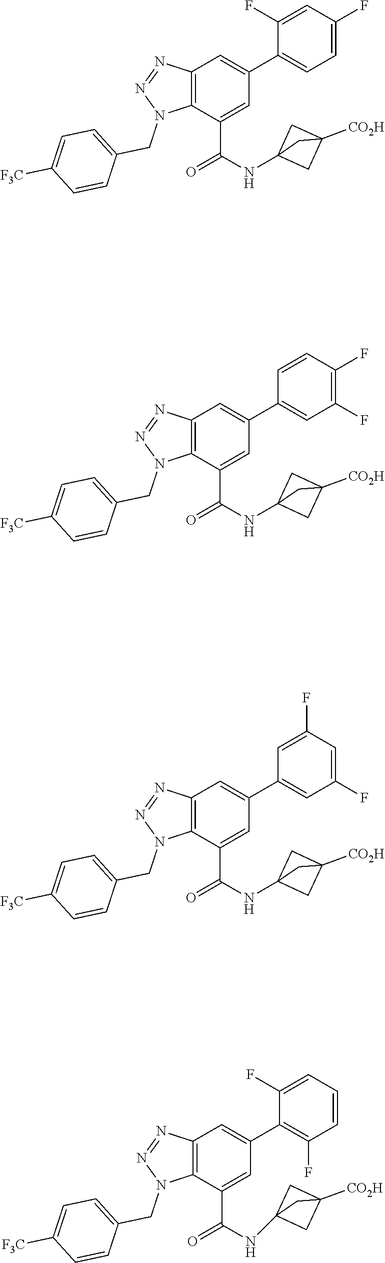 [SMILES: O=C(NC12CC(C(=O)O)(C1)C2)c1cc(-c2ccc(F)cc2F)cc2nnn(Cc3ccc(C(F)(F)F)cc3)c12 ; O=C(NC12CC(C(=O)O)(C1)C2)c1cc(-c2ccc(F)c(F)c2)cc2nnn(Cc3ccc(C(F)(F)F)cc3)c12 ; O=C(NC12CC(C(=O)O)(C1)C2)c1cc(-c2cc(F)cc(F)c2)cc2nnn(Cc3ccc(C(F)(F)F)cc3)c12 ; O=C(NC12CC(C(=O)O)(C1)C2)c1cc(-c2c(F)cccc2F)cc2nnn(Cc3ccc(C(F)(F)F)cc3)c12]

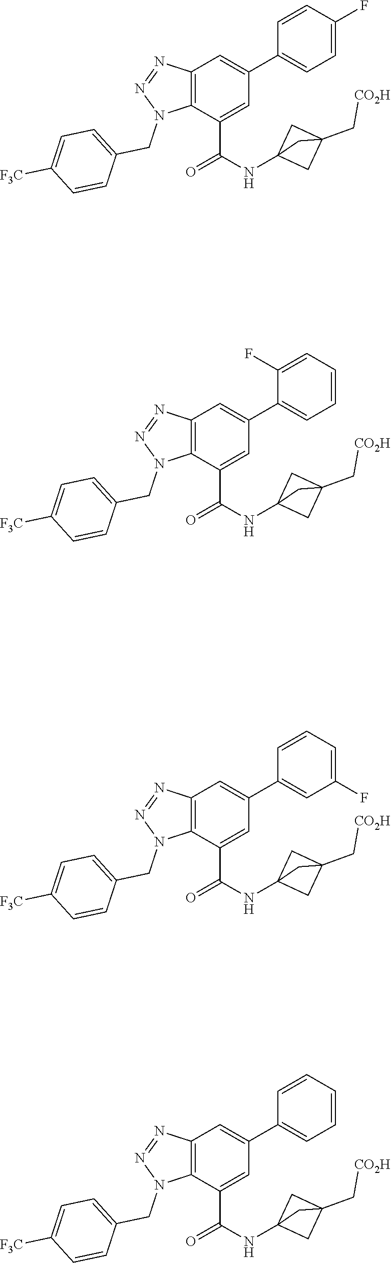 [SMILES: O=C(O)CC12CC(NC(=O)c3cc(-c4ccc(F)cc4)cc4nnn(Cc5ccc(C(F)(F)F)cc5)c34)(C1)C2 ; O=C(O)CC12CC(NC(=O)c3cc(-c4ccccc4F)cc4nnn(Cc5ccc(C(F)(F)F)cc5)c34)(C1)C2 ; O=C(O)CC12CC(NC(=O)c3cc(-c4cccc(F)c4)cc4nnn(Cc5ccc(C(F)(F)F)cc5)c34)(C1)C2 ; O=C(O)CC12CC(NC(=O)c3cc(-c4ccccc4)cc4nnn(Cc5ccc(C(F)(F)F)cc5)c34)(C1)C2]

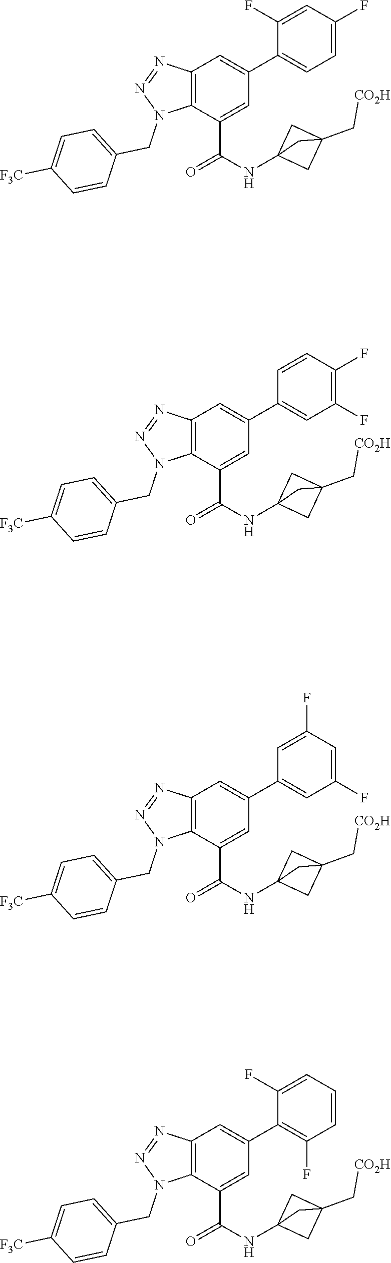 [SMILES: O=C(O)CC12CC(NC(=O)c3cc(-c4ccc(F)cc4F)cc4nnn(Cc5ccc(C(F)(F)F)cc5)c34)(C1)C2 ; O=C(O)CC12CC(NC(=O)c3cc(-c4ccc(F)c(F)c4)cc4nnn(Cc5ccc(C(F)(F)F)cc5)c34)(C1)C2 ; O=C(O)CC12CC(NC(=O)c3cc(-c4cc(F)cc(F)c4)cc4nnn(Cc5ccc(C(F)(F)F)cc5)c34)(C1)C2 ; O=C(O)CC12CC(NC(=O)c3cc(-c4c(F)cccc4F)cc4nnn(Cc5ccc(C(F)(F)F)cc5)c34)(C1)C2]

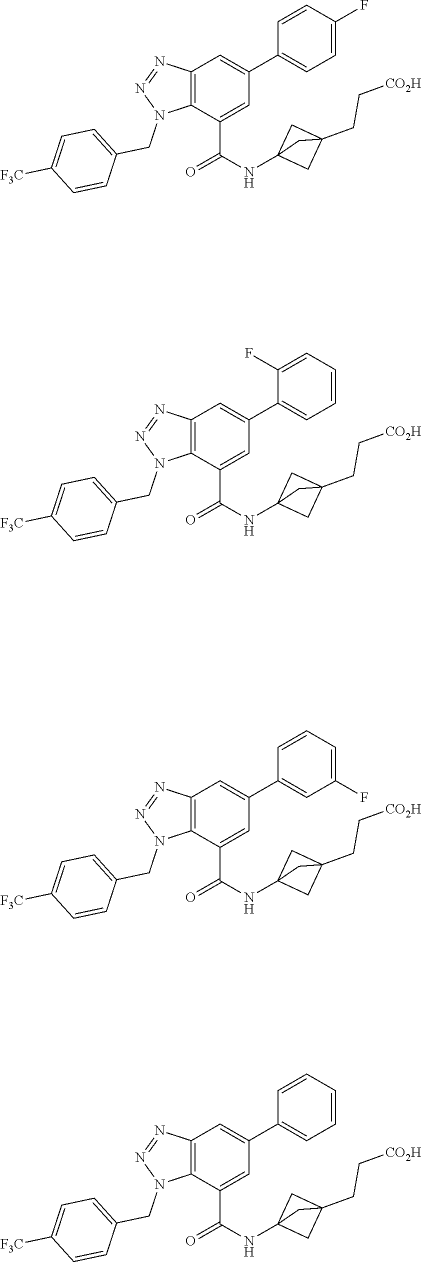 [SMILES: O=C(O)CCC12CC(NC(=O)c3cc(-c4ccc(F)cc4)cc4nnn(Cc5ccc(C(F)(F)F)cc5)c34)(C1)C2 ; O=C(O)CCC12CC(NC(=O)c3cc(-c4ccccc4F)cc4nnn(Cc5ccc(C(F)(F)F)cc5)c34)(C1)C2 ; O=C(O)CCC12CC(NC(=O)c3cc(-c4cccc(F)c4)cc4nnn(Cc5ccc(C(F)(F)F)cc5)c34)(C1)C2 ; O=C(O)CCC12CC(NC(=O)c3cc(-c4ccccc4)cc4nnn(Cc5ccc(C(F)(F)F)cc5)c34)(C1)C2]

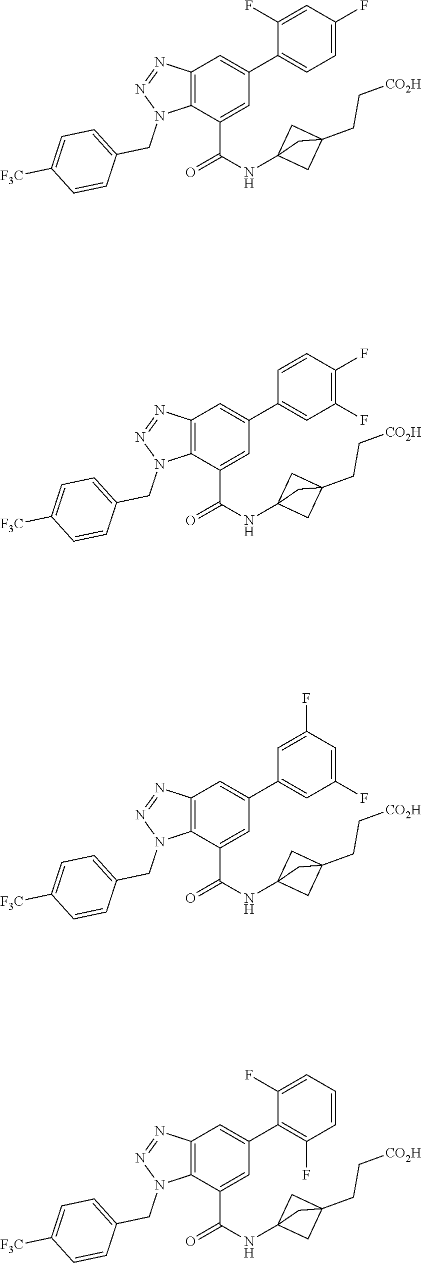 [SMILES: O=C(O)CCC12CC(NC(=O)c3cc(-c4ccc(F)cc4F)cc4nnn(Cc5ccc(C(F)(F)F)cc5)c34)(C1)C2 ; O=C(O)CCC12CC(NC(=O)c3cc(-c4ccc(F)c(F)c4)cc4nnn(Cc5ccc(C(F)(F)F)cc5)c34)(C1)C2 ; O=C(O)CCC12CC(NC(=O)c3cc(-c4cc(F)cc(F)c4)cc4nnn(Cc5ccc(C(F)(F)F)cc5)c34)(C1)C2 ; O=C(O)CCC12CC(NC(=O)c3cc(-c4c(F)cccc4F)cc4nnn(Cc5ccc(C(F)(F)F)cc5)c34)(C1)C2]

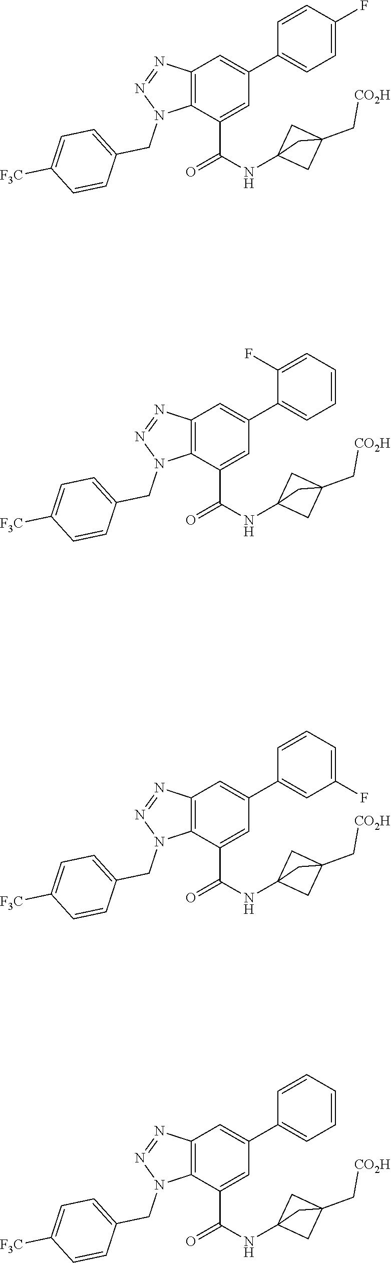 [SMILES: O=C(O)CC12CC(NC(=O)c3cc(-c4ccc(F)cc4)cc4nnn(Cc5ccc(C(F)(F)F)cc5)c34)(C1)C2 ; O=C(O)CC12CC(NC(=O)c3cc(-c4ccccc4F)cc4nnn(Cc5ccc(C(F)(F)F)cc5)c34)(C1)C2 ; O=C(O)CC12CC(NC(=O)c3cc(-c4cccc(F)c4)cc4nnn(Cc5ccc(C(F)(F)F)cc5)c34)(C1)C2 ; O=C(O)CC12CC(NC(=O)c3cc(-c4ccccc4)cc4nnn(Cc5ccc(C(F)(F)F)cc5)c34)(C1)C2]

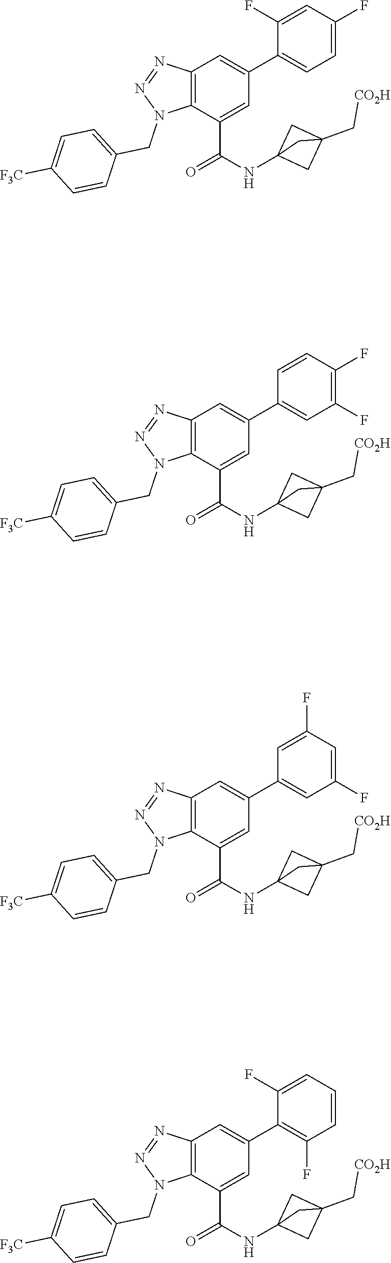 [SMILES: O=C(O)CC12CC(NC(=O)c3cc(-c4ccc(F)cc4F)cc4nnn(Cc5ccc(C(F)(F)F)cc5)c34)(C1)C2 ; O=C(O)CC12CC(NC(=O)c3cc(-c4ccc(F)c(F)c4)cc4nnn(Cc5ccc(C(F)(F)F)cc5)c34)(C1)C2 ; O=C(O)CC12CC(NC(=O)c3cc(-c4cc(F)cc(F)c4)cc4nnn(Cc5ccc(C(F)(F)F)cc5)c34)(C1)C2 ; O=C(O)CC12CC(NC(=O)c3cc(-c4c(F)cccc4F)cc4nnn(Cc5ccc(C(F)(F)F)cc5)c34)(C1)C2]

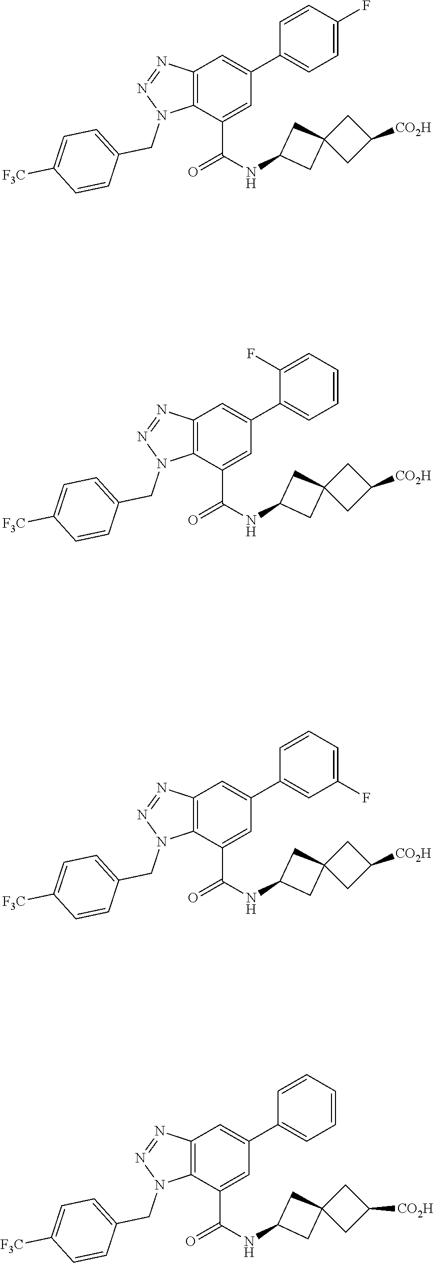

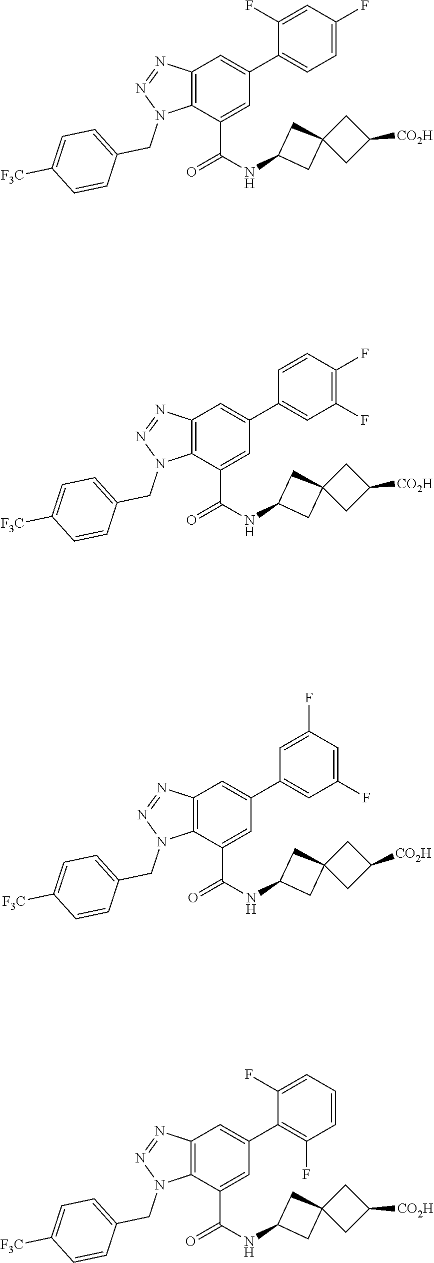

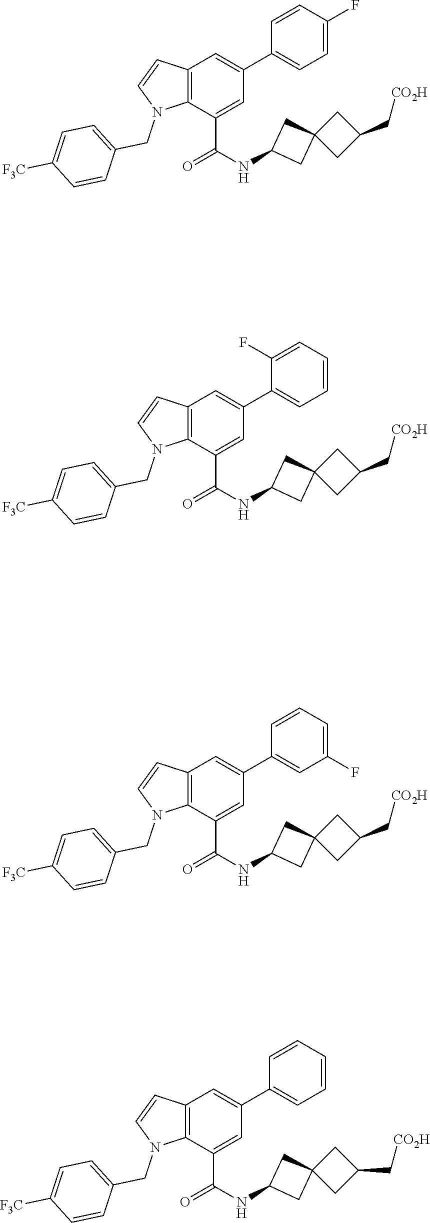

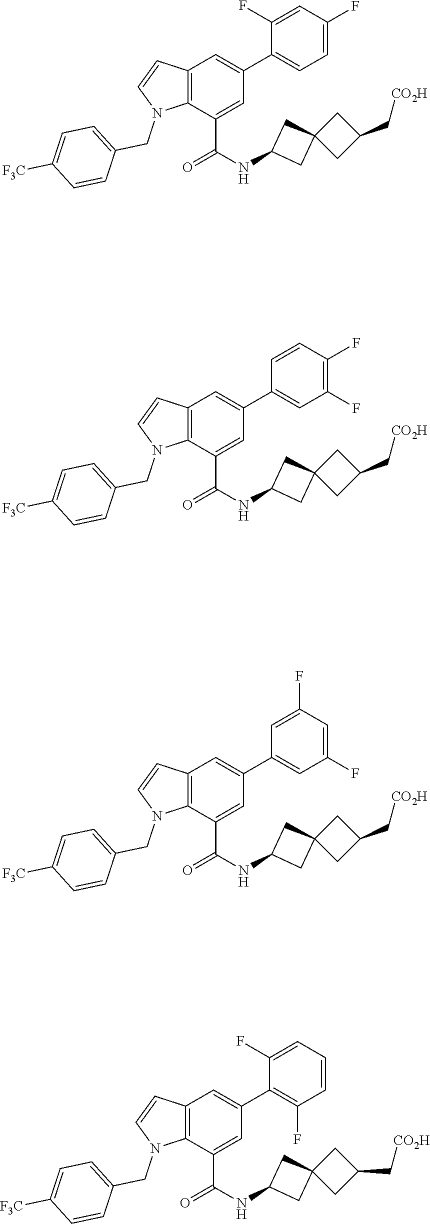

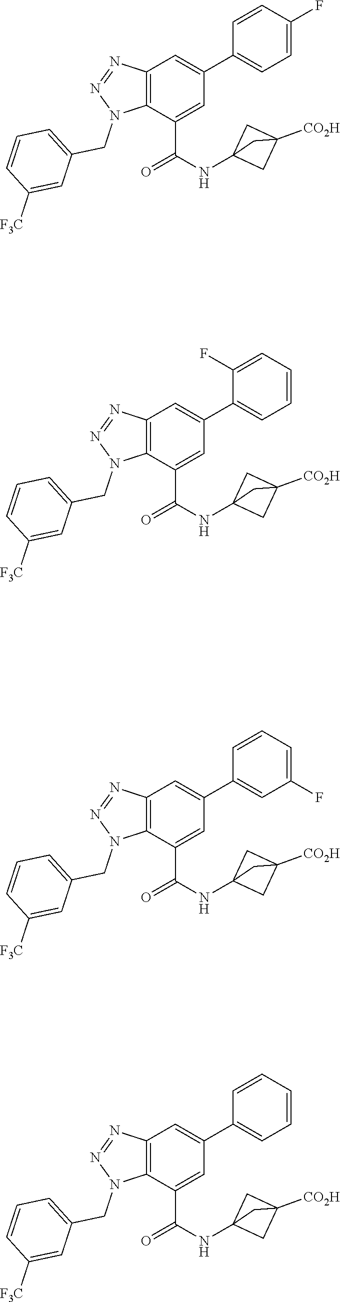 [SMILES: O=C(NC12CC(C(=O)O)(C1)C2)c1cc(-c2ccc(F)cc2)cc2nnn(Cc3cccc(C(F)(F)F)c3)c12 ; O=C(NC12CC(C(=O)O)(C1)C2)c1cc(-c2ccccc2F)cc2nnn(Cc3cccc(C(F)(F)F)c3)c12 ; O=C(NC12CC(C(=O)O)(C1)C2)c1cc(-c2cccc(F)c2)cc2nnn(Cc3cccc(C(F)(F)F)c3)c12 ; O=C(NC12CC(C(=O)O)(C1)C2)c1cc(-c2ccccc2)cc2nnn(Cc3cccc(C(F)(F)F)c3)c12]

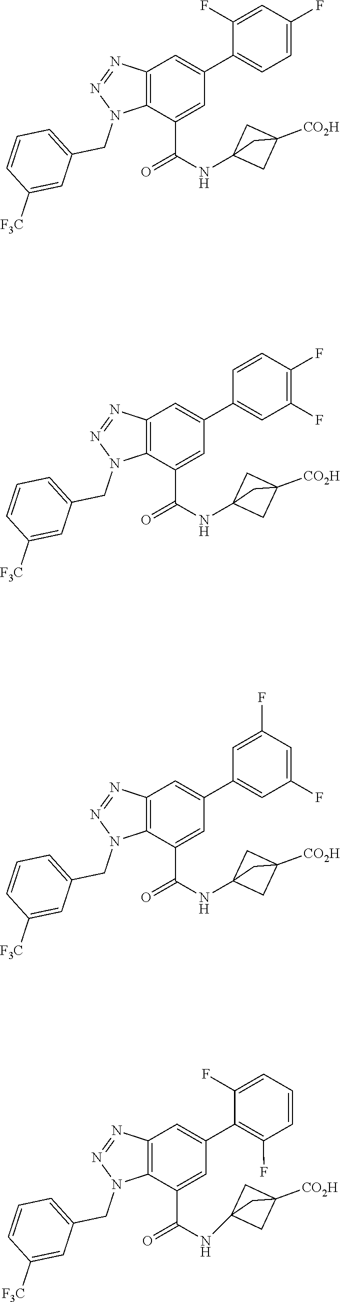 [SMILES: O=C(NC12CC(C(=O)O)(C1)C2)c1cc(-c2ccc(F)cc2F)cc2nnn(Cc3cccc(C(F)(F)F)c3)c12 ; O=C(NC12CC(C(=O)O)(C1)C2)c1cc(-c2ccc(F)c(F)c2)cc2nnn(Cc3cccc(C(F)(F)F)c3)c12 ; O=C(NC12CC(C(=O)O)(C1)C2)c1cc(-c2cc(F)cc(F)c2)cc2nnn(Cc3cccc(C(F)(F)F)c3)c12 ; O=C(NC12CC(C(=O)O)(C1)C2)c1cc(-c2c(F)cccc2F)cc2nnn(Cc3cccc(C(F)(F)F)c3)c12]

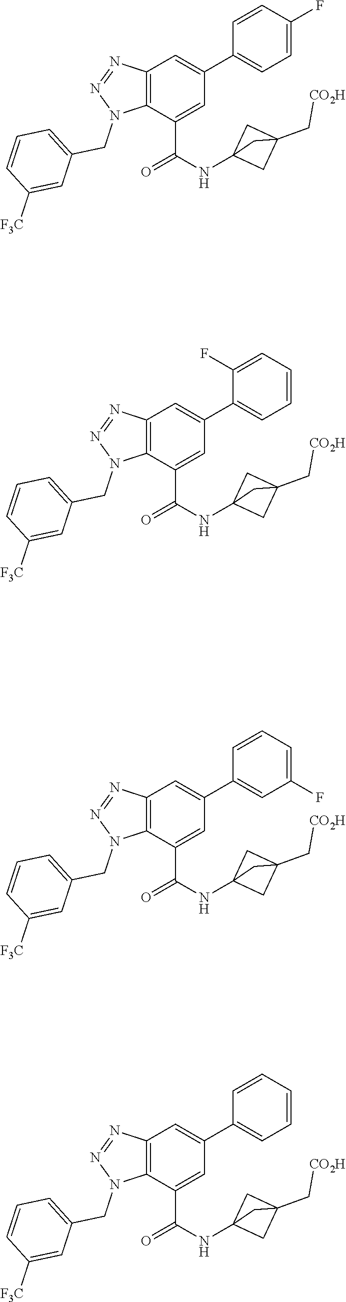 [SMILES: O=C(O)CC12CC(NC(=O)c3cc(-c4ccc(F)cc4)cc4nnn(Cc5cccc(C(F)(F)F)c5)c34)(C1)C2 ; O=C(O)CC12CC(NC(=O)c3cc(-c4ccccc4F)cc4nnn(Cc5cccc(C(F)(F)F)c5)c34)(C1)C2 ; O=C(O)CC12CC(NC(=O)c3cc(-c4cccc(F)c4)cc4nnn(Cc5cccc(C(F)(F)F)c5)c34)(C1)C2 ; O=C(O)CC12CC(NC(=O)c3cc(-c4ccccc4)cc4nnn(Cc5cccc(C(F)(F)F)c5)c34)(C1)C2]

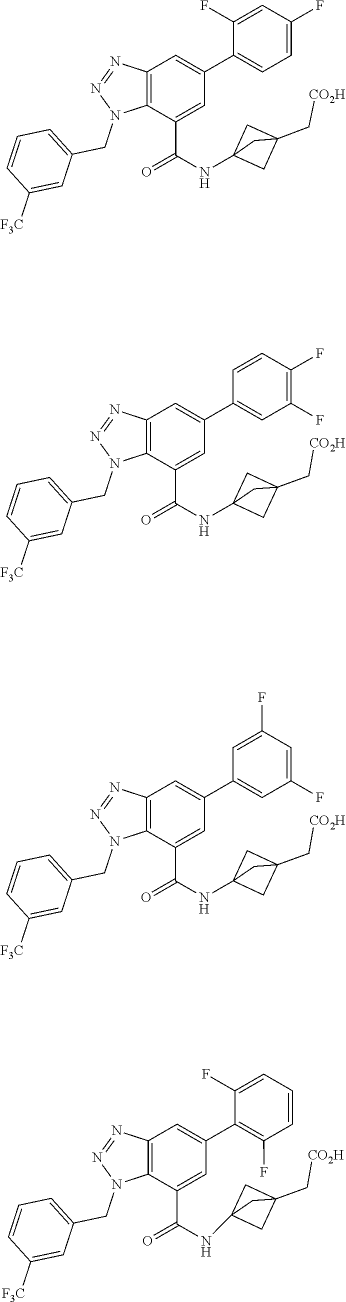 [SMILES: O=C(O)CC12CC(NC(=O)c3cc(-c4ccc(F)cc4F)cc4nnn(Cc5cccc(C(F)(F)F)c5)c34)(C1)C2 ; O=C(O)CC12CC(NC(=O)c3cc(-c4ccc(F)c(F)c4)cc4nnn(Cc5cccc(C(F)(F)F)c5)c34)(C1)C2 ; O=C(O)CC12CC(NC(=O)c3cc(-c4cc(F)cc(F)c4)cc4nnn(Cc5cccc(C(F)(F)F)c5)c34)(C1)C2 ; O=C(O)CC12CC(NC(=O)c3cc(-c4c(F)cccc4F)cc4nnn(Cc5cccc(C(F)(F)F)c5)c34)(C1)C2]

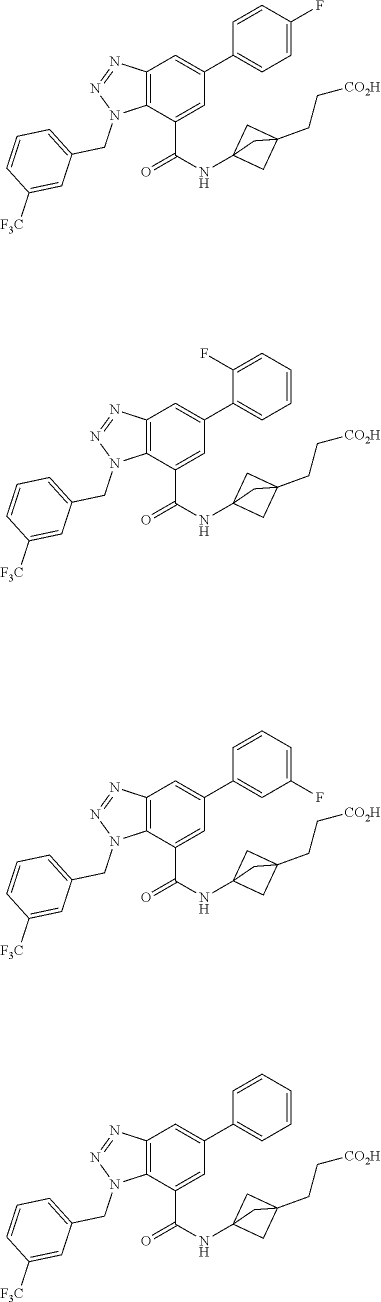 [SMILES: O=C(O)CCC12CC(NC(=O)c3cc(-c4ccc(F)cc4)cc4nnn(Cc5cccc(C(F)(F)F)c5)c34)(C1)C2 ; O=C(O)CCC12CC(NC(=O)c3cc(-c4ccccc4F)cc4nnn(Cc5cccc(C(F)(F)F)c5)c34)(C1)C2 ; O=C(O)CCC12CC(NC(=O)c3cc(-c4cccc(F)c4)cc4nnn(Cc5cccc(C(F)(F)F)c5)c34)(C1)C2 ; O=C(O)CCC12CC(NC(=O)c3cc(-c4ccccc4)cc4nnn(Cc5cccc(C(F)(F)F)c5)c34)(C1)C2]

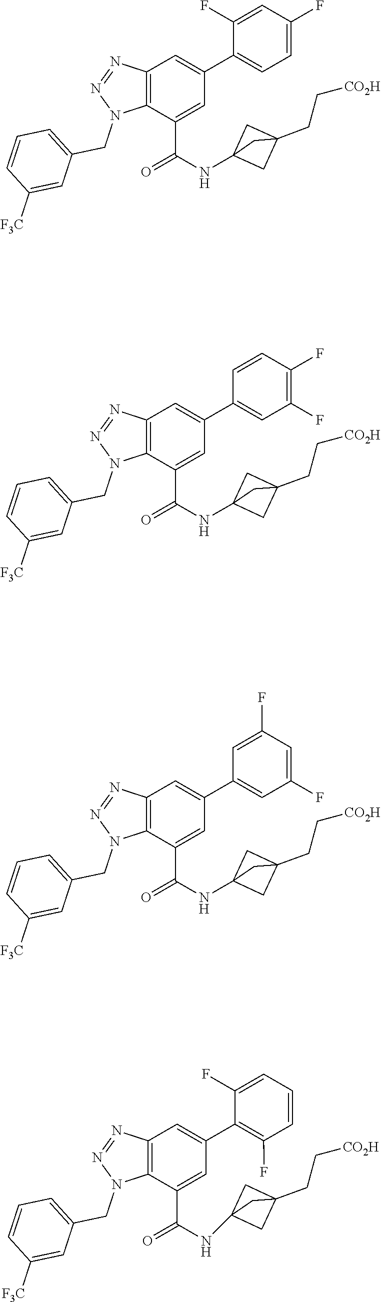 [SMILES: O=C(O)CCC12CC(NC(=O)c3cc(-c4ccc(F)cc4F)cc4nnn(Cc5cccc(C(F)(F)F)c5)c34)(C1)C2 ; O=C(O)CCC12CC(NC(=O)c3cc(-c4ccc(F)c(F)c4)cc4nnn(Cc5cccc(C(F)(F)F)c5)c34)(C1)C2 ; O=C(O)CCC12CC(NC(=O)c3cc(-c4cc(F)cc(F)c4)cc4nnn(Cc5cccc(C(F)(F)F)c5)c34)(C1)C2 ; O=C(O)CCC12CC(NC(=O)c3cc(-c4c(F)cccc4F)cc4nnn(Cc5cccc(C(F)(F)F)c5)c34)(C1)C2]

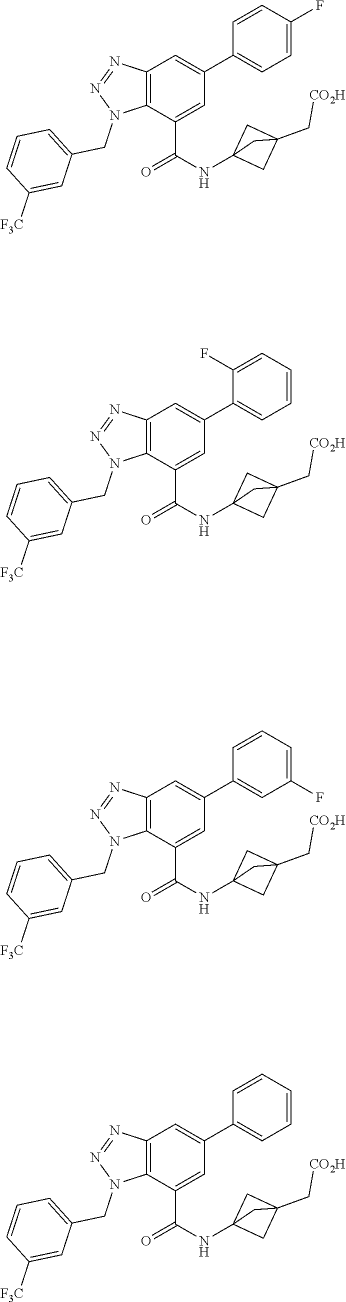 [SMILES: O=C(O)CC12CC(NC(=O)c3cc(-c4ccc(F)cc4)cc4nnn(Cc5cccc(C(F)(F)F)c5)c34)(C1)C2 ; O=C(O)CC12CC(NC(=O)c3cc(-c4ccccc4F)cc4nnn(Cc5cccc(C(F)(F)F)c5)c34)(C1)C2 ; O=C(O)CC12CC(NC(=O)c3cc(-c4cccc(F)c4)cc4nnn(Cc5cccc(C(F)(F)F)c5)c34)(C1)C2 ; O=C(O)CC12CC(NC(=O)c3cc(-c4ccccc4)cc4nnn(Cc5cccc(C(F)(F)F)c5)c34)(C1)C2]

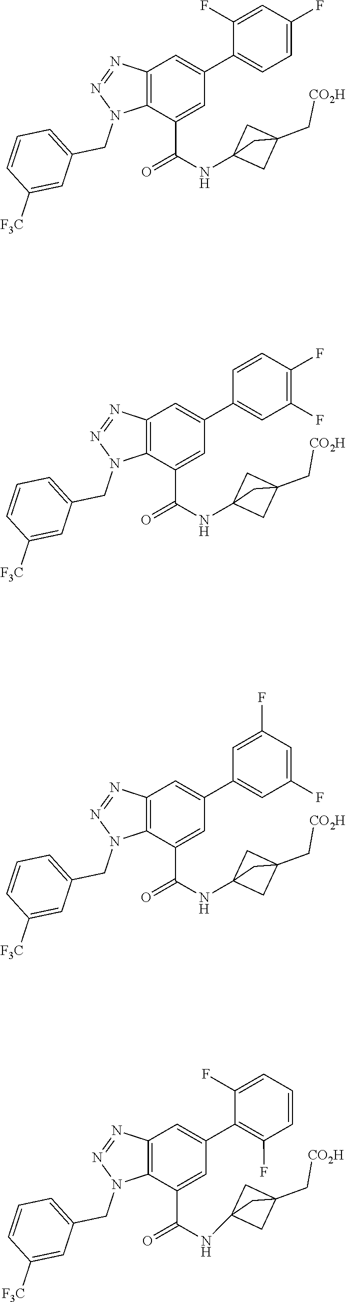 [SMILES: O=C(O)CC12CC(NC(=O)c3cc(-c4ccc(F)cc4F)cc4nnn(Cc5cccc(C(F)(F)F)c5)c34)(C1)C2 ; O=C(O)CC12CC(NC(=O)c3cc(-c4ccc(F)c(F)c4)cc4nnn(Cc5cccc(C(F)(F)F)c5)c34)(C1)C2 ; O=C(O)CC12CC(NC(=O)c3cc(-c4cc(F)cc(F)c4)cc4nnn(Cc5cccc(C(F)(F)F)c5)c34)(C1)C2 ; O=C(O)CC12CC(NC(=O)c3cc(-c4c(F)cccc4F)cc4nnn(Cc5cccc(C(F)(F)F)c5)c34)(C1)C2]

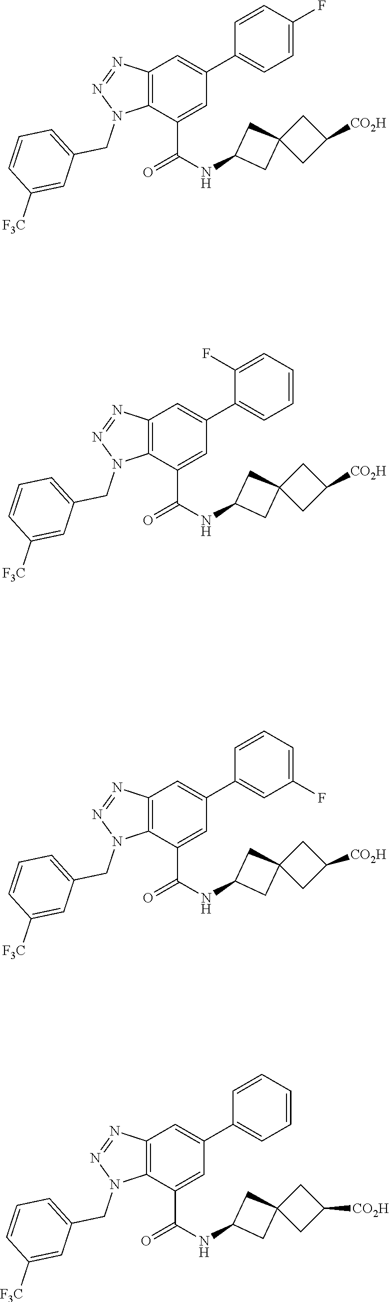

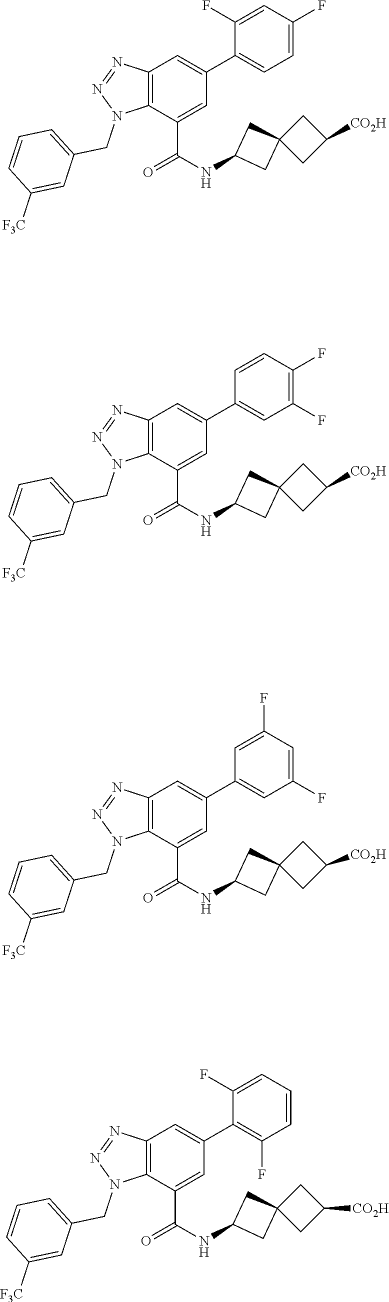

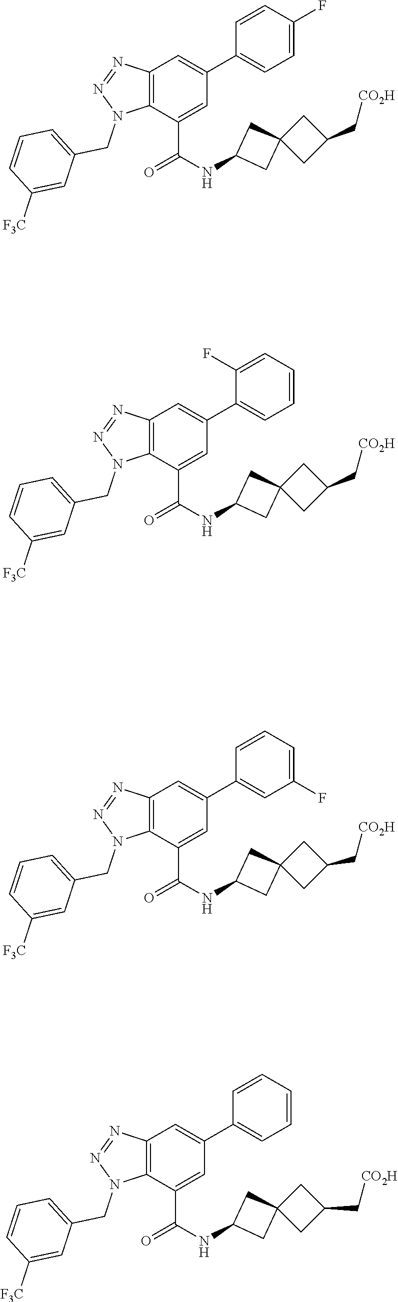

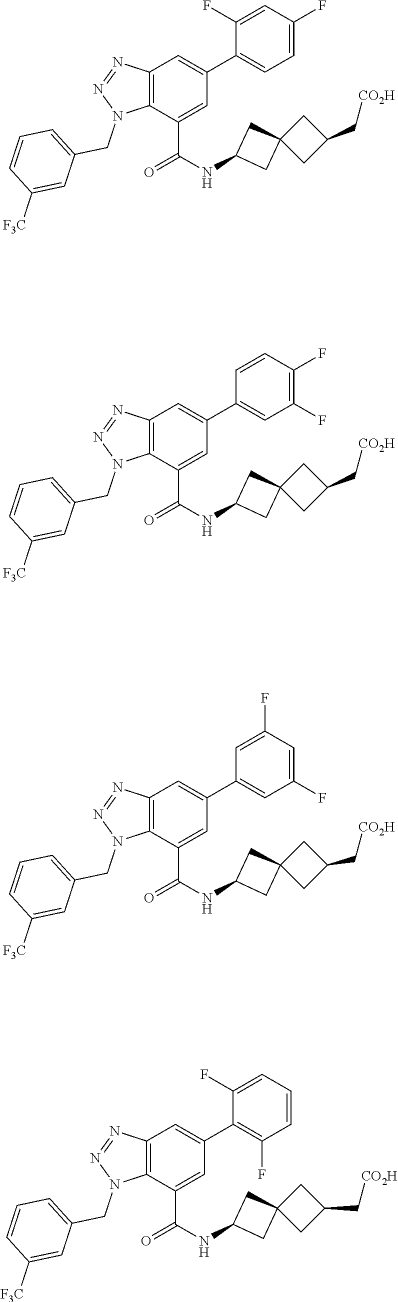

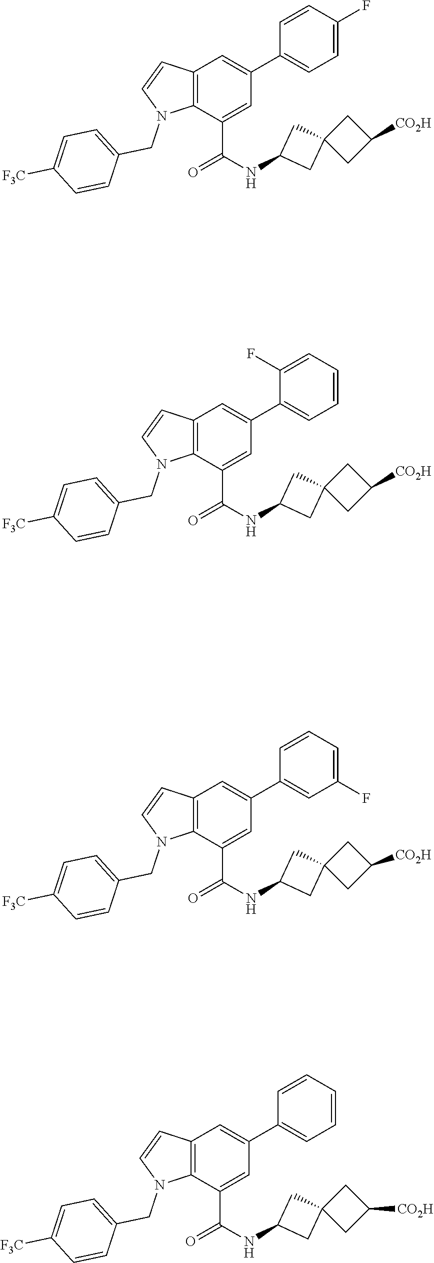

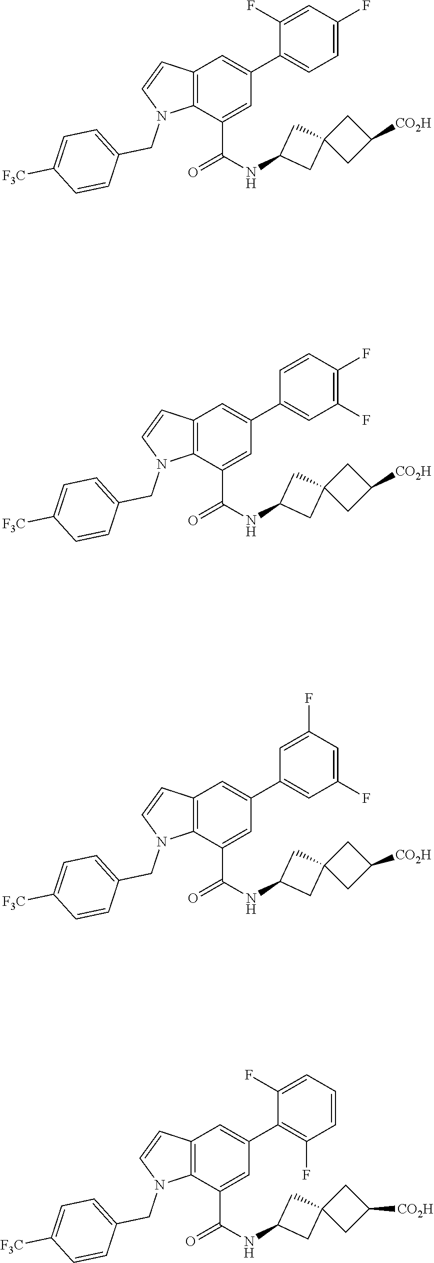

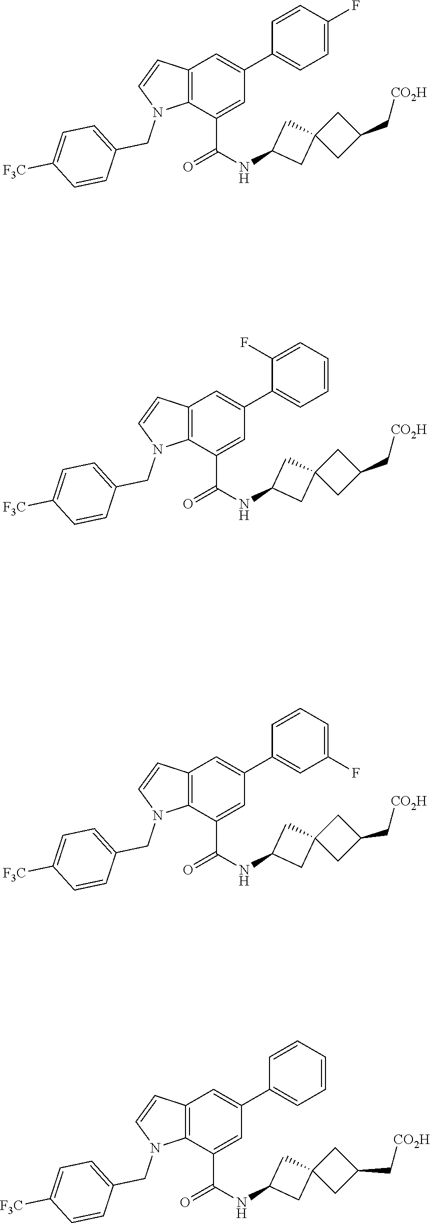

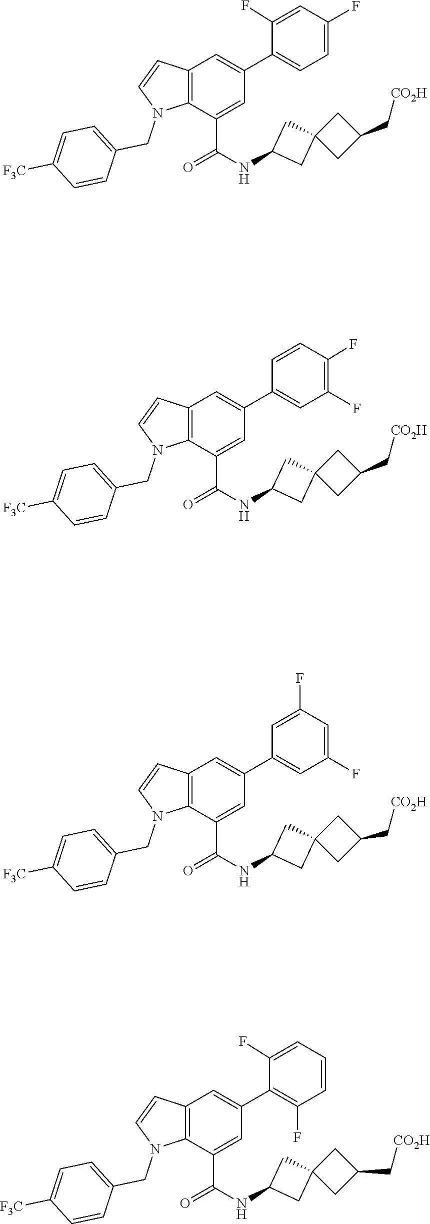

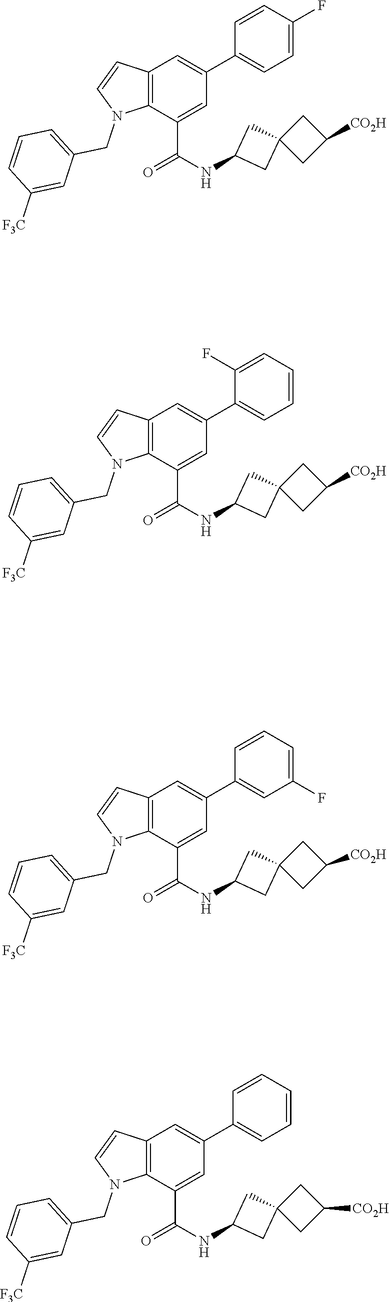

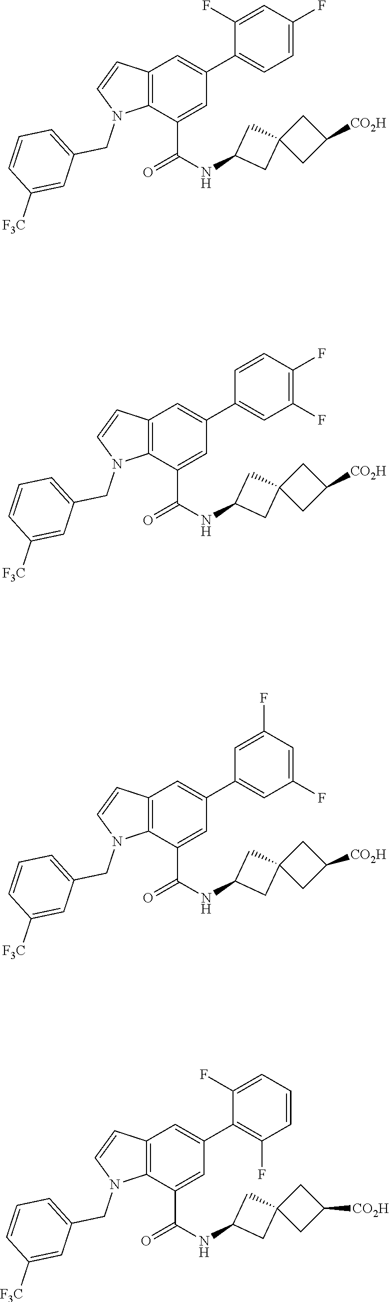

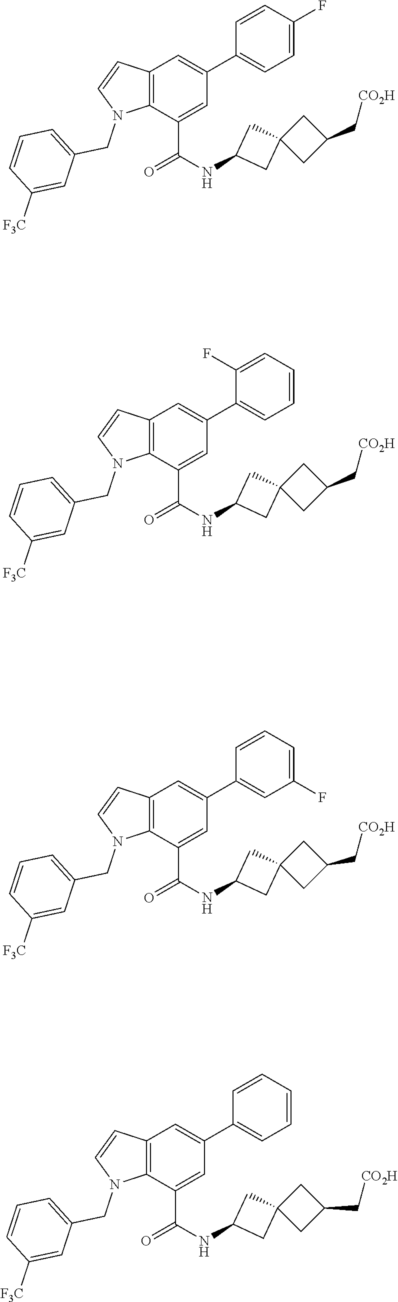

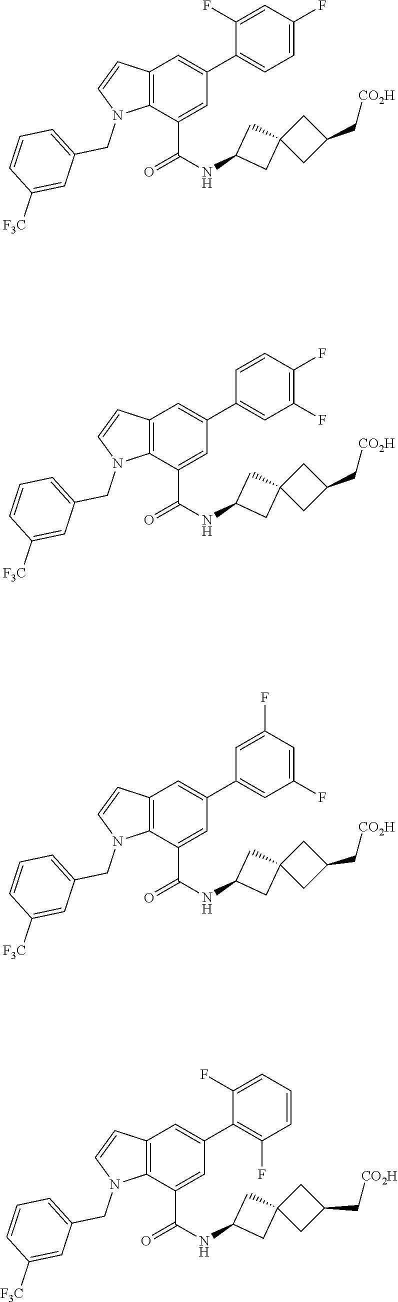

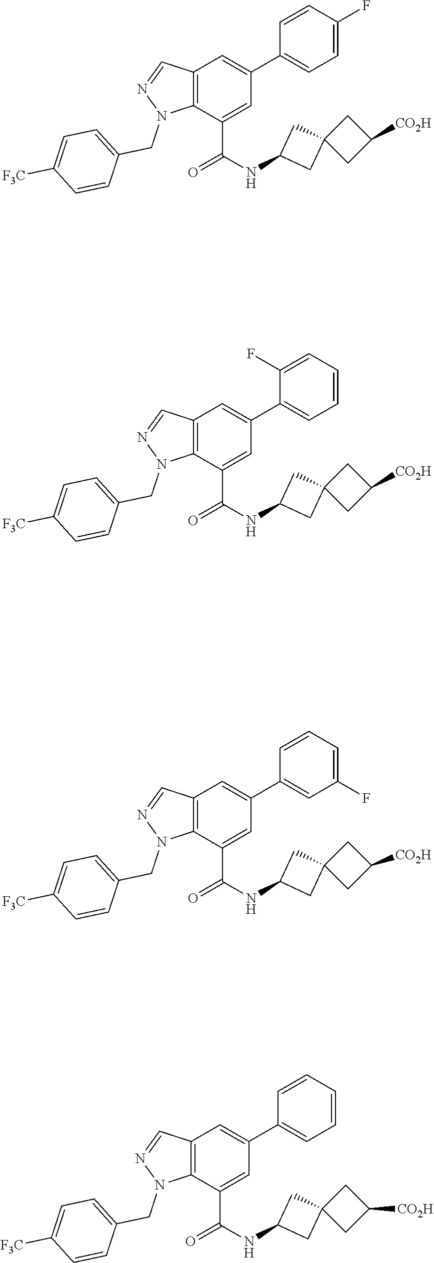

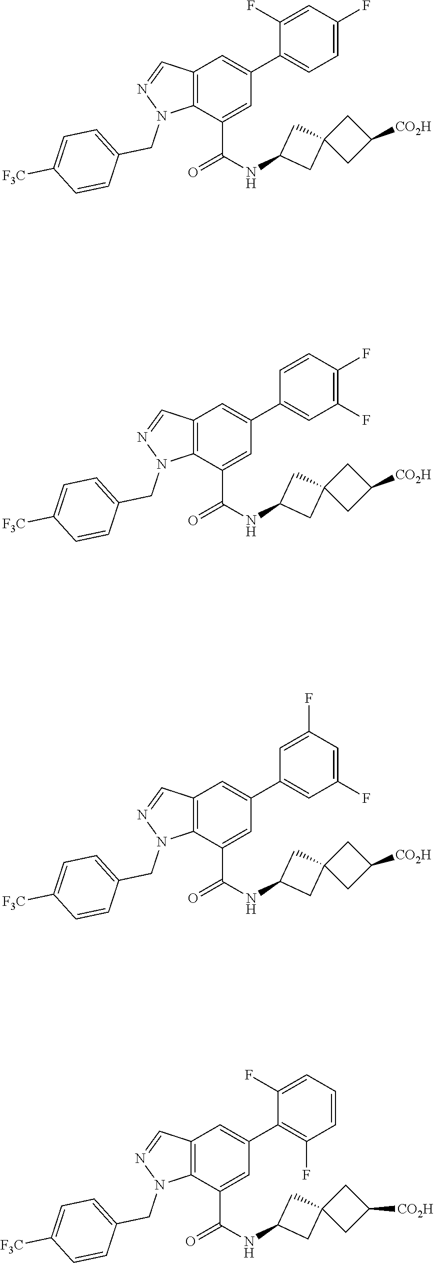

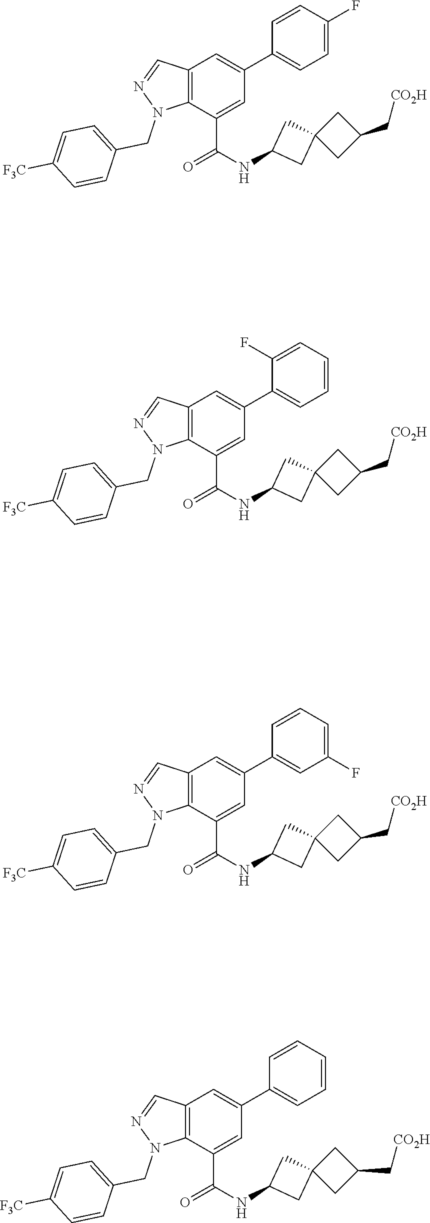

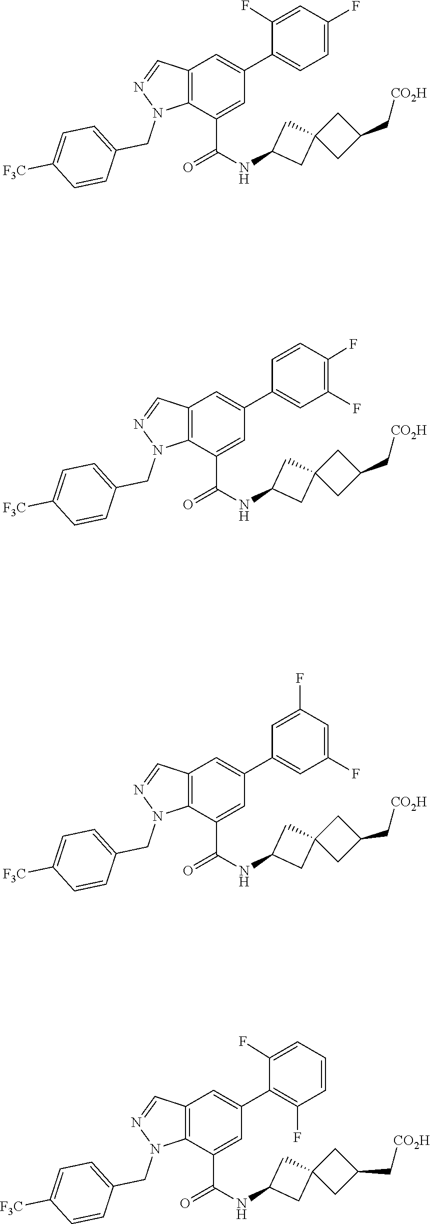

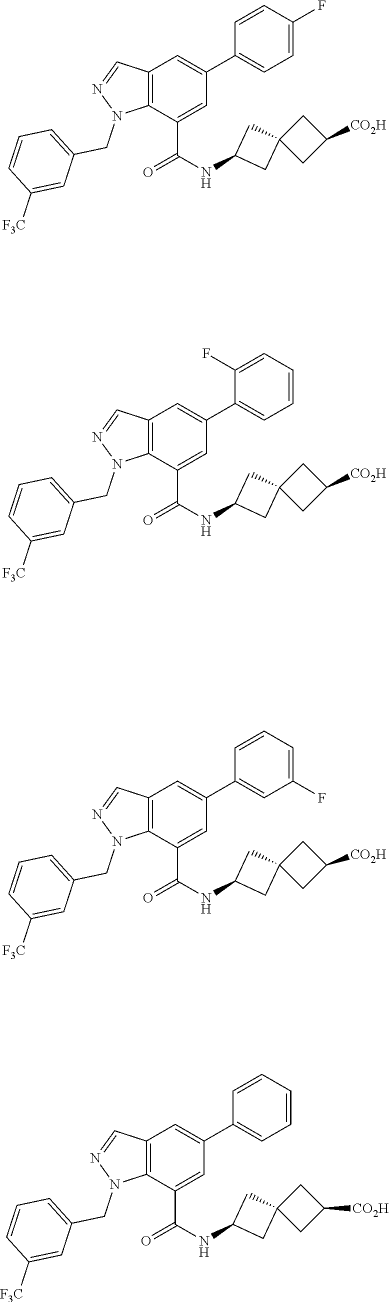

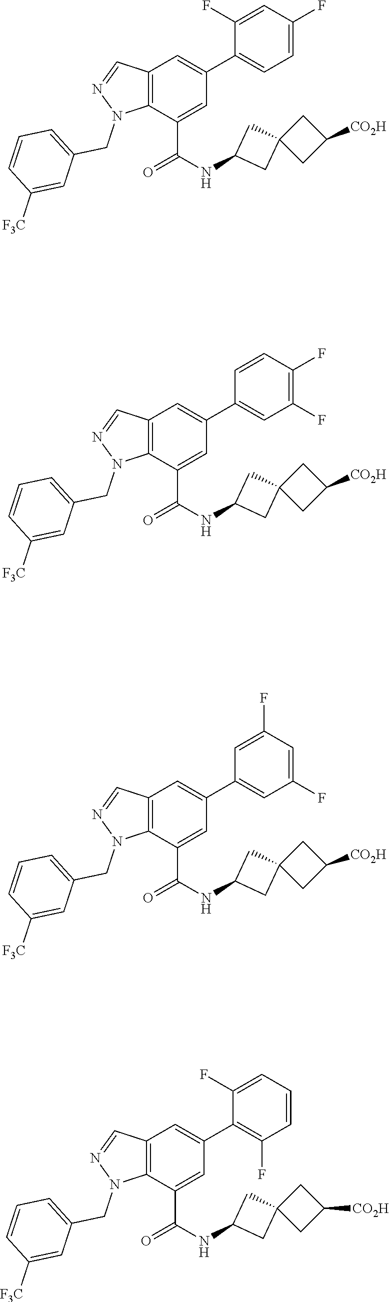

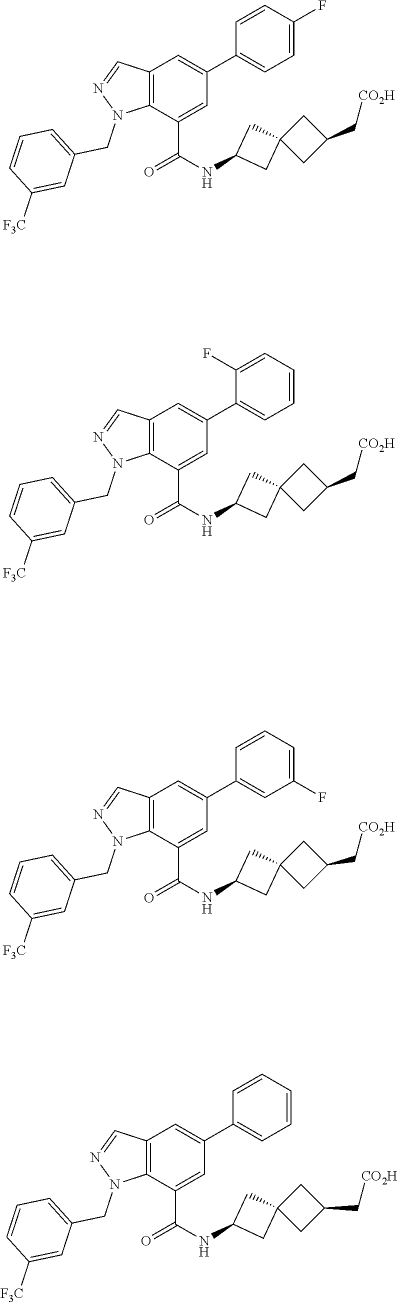

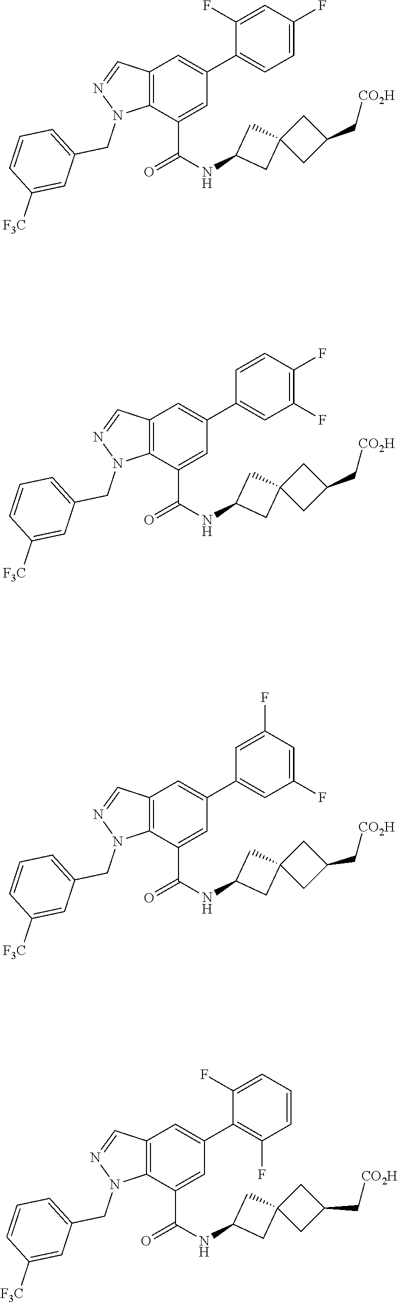

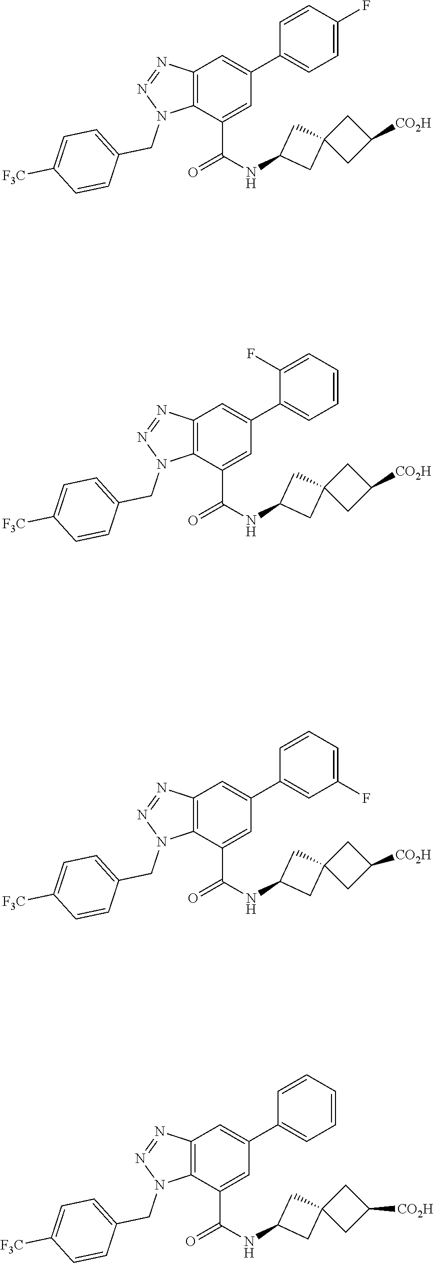

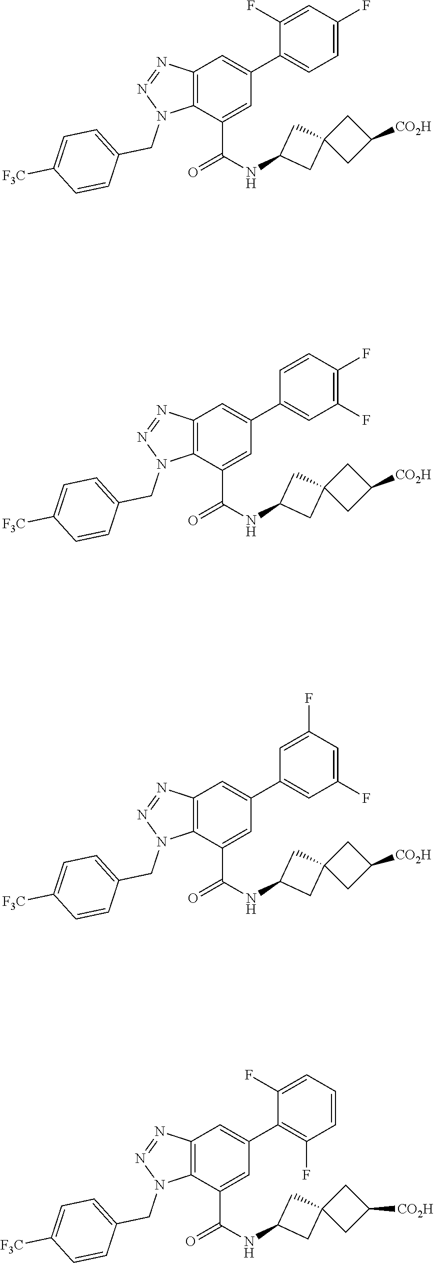

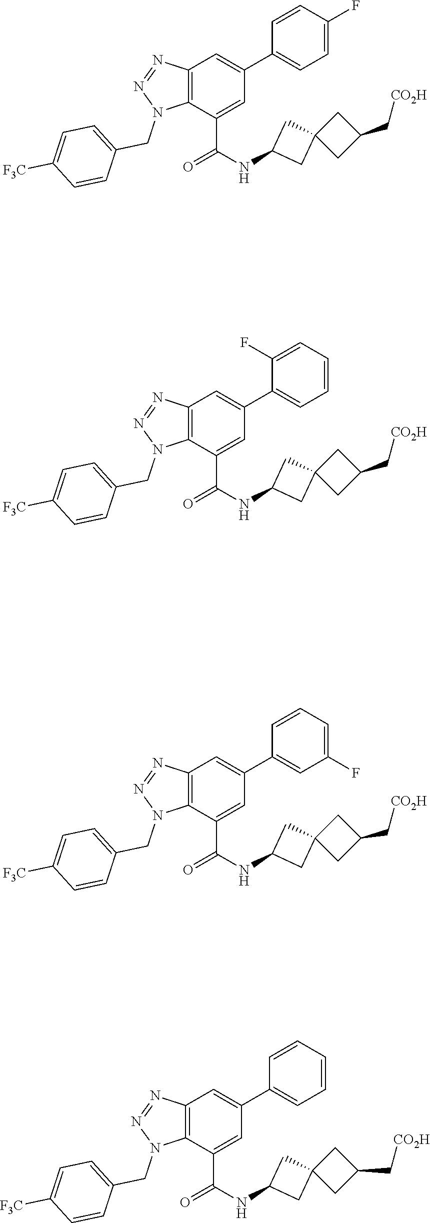

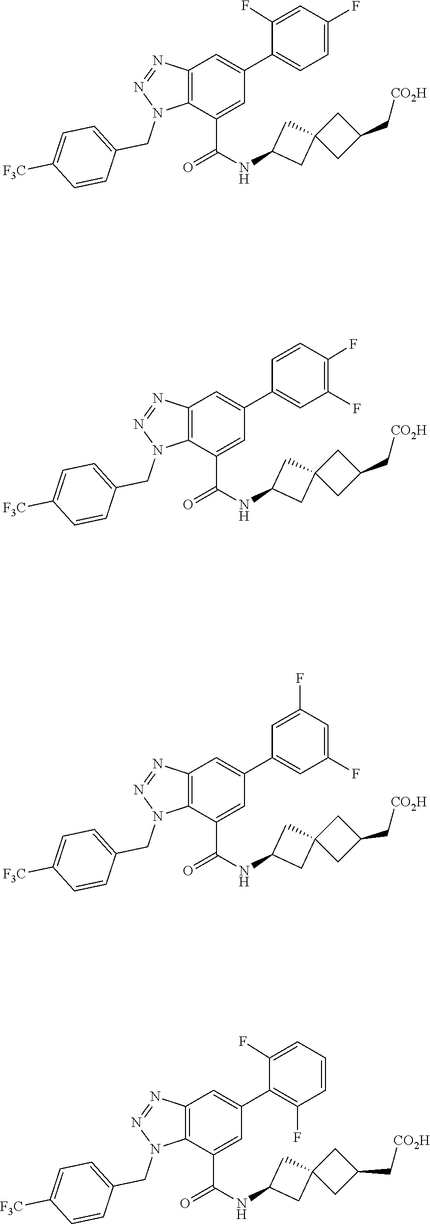

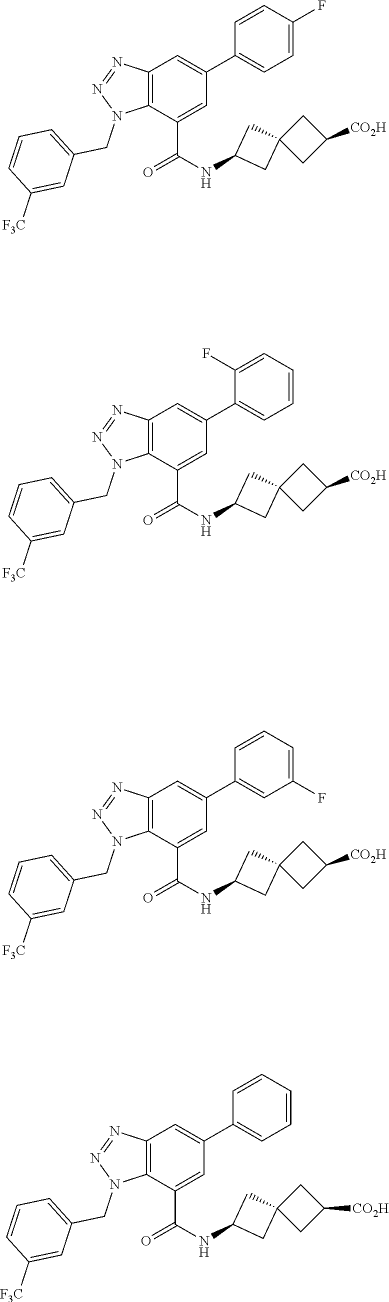

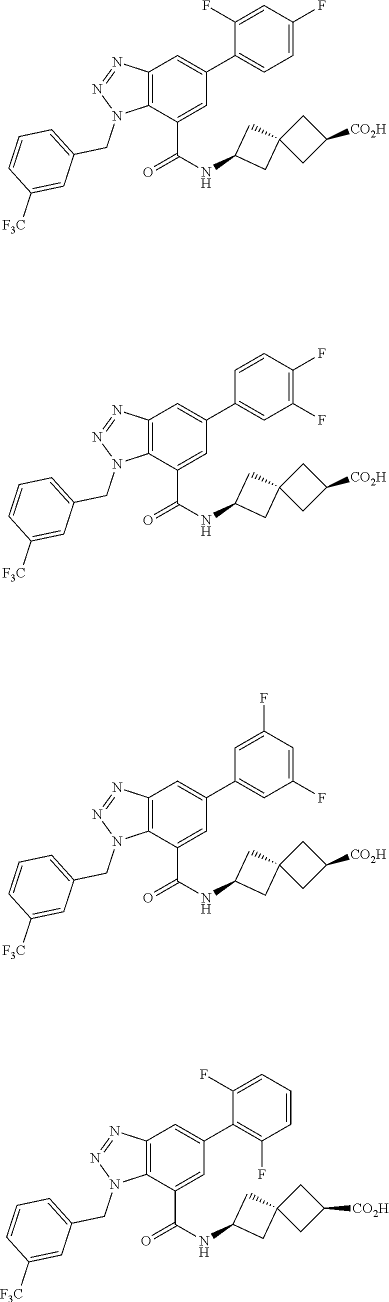

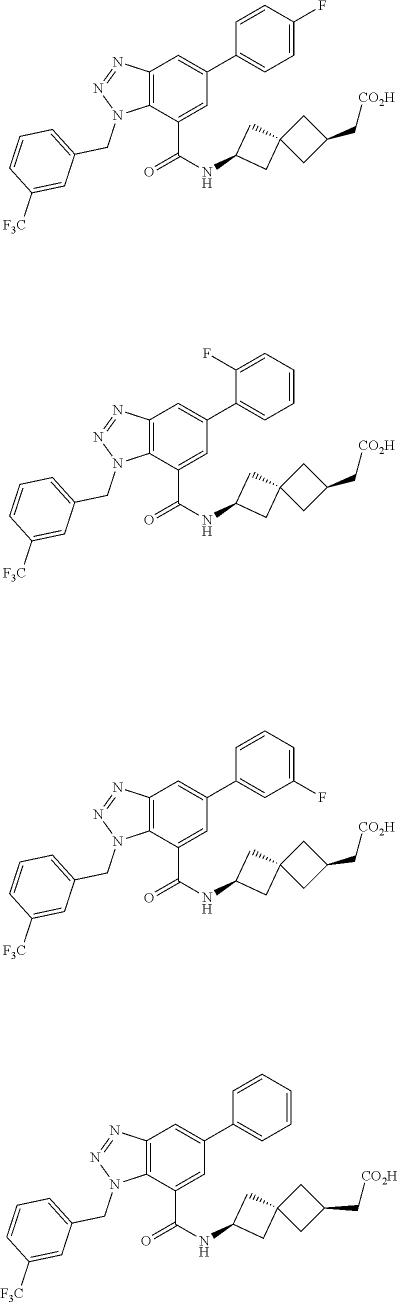

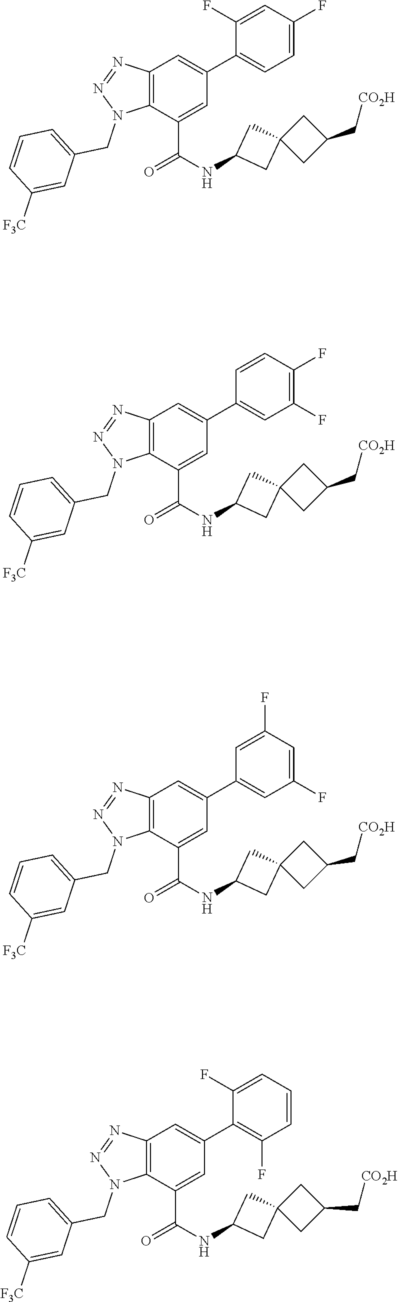

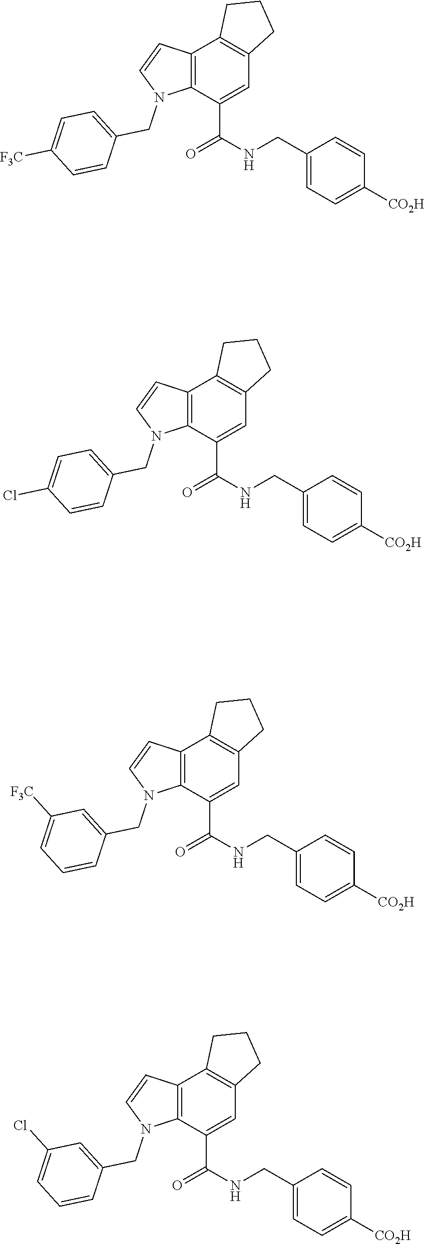 [SMILES: O=C(O)c1ccc(CNC(=O)c2cc3c(c4ccn(Cc5ccc(C(F)(F)F)cc5)c24)CCC3)cc1 ; O=C(O)c1ccc(CNC(=O)c2cc3c(c4ccn(Cc5ccc(Cl)cc5)c24)CCC3)cc1 ; O=C(O)c1ccc(CNC(=O)c2cc3c(c4ccn(Cc5cccc(C(F)(F)F)c5)c24)CCC3)cc1 ; O=C(O)c1ccc(CNC(=O)c2cc3c(c4ccn(Cc5cccc(Cl)c5)c24)CCC3)cc1]

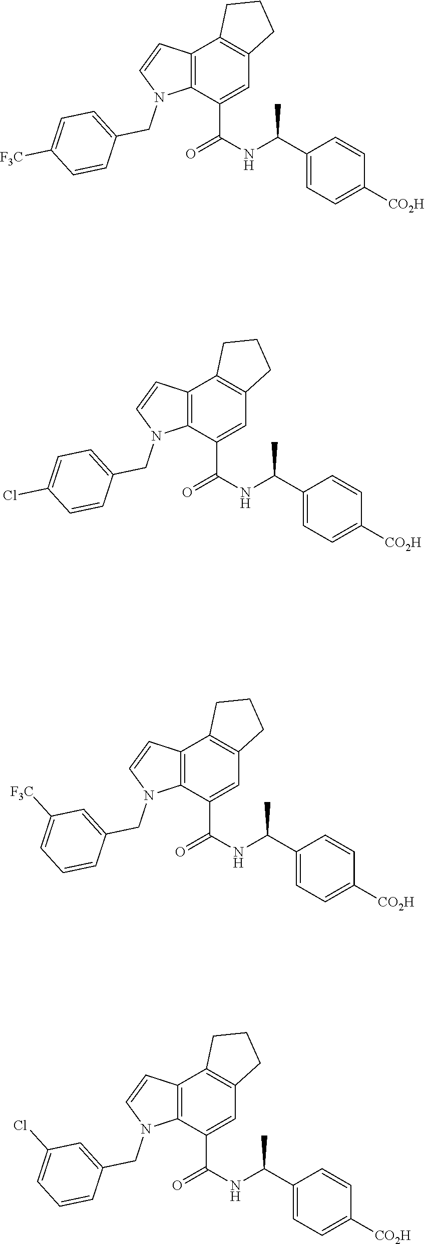 [SMILES: C[C@H](NC(=O)c1cc2c(c3ccn(Cc4ccc(C(F)(F)F)cc4)c13)CCC2)c1ccc(C(=O)O)cc1 ; C[C@H](NC(=O)c1cc2c(c3ccn(Cc4ccc(Cl)cc4)c13)CCC2)c1ccc(C(=O)O)cc1 ; C[C@H](NC(=O)c1cc2c(c3ccn(Cc4cccc(C(F)(F)F)c4)c13)CCC2)c1ccc(C(=O)O)cc1 ; C[C@H](NC(=O)c1cc2c(c3ccn(Cc4cccc(Cl)c4)c13)CCC2)c1ccc(C(=O)O)cc1]

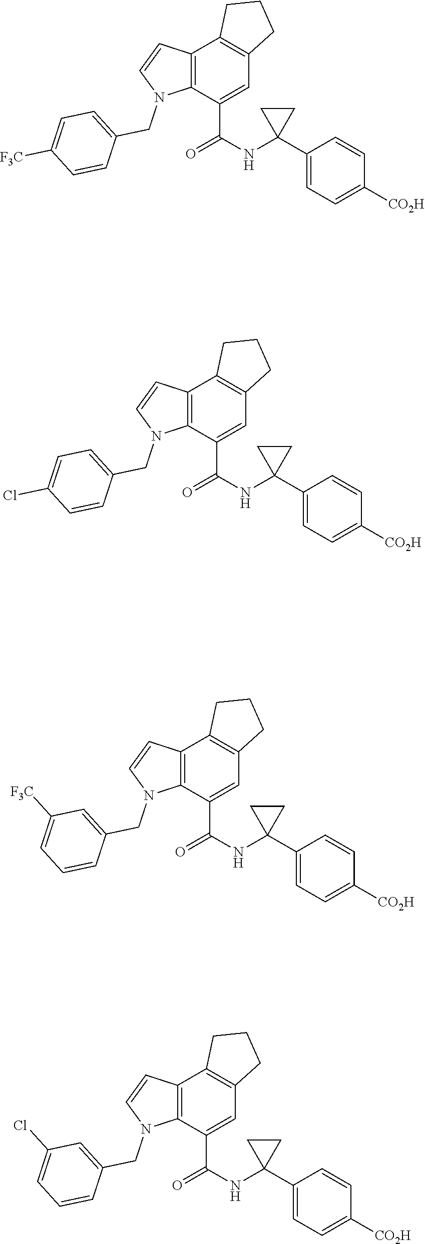 [SMILES: O=C(O)c1ccc(C2(NC(=O)c3cc4c(c5ccn(Cc6ccc(C(F)(F)F)cc6)c35)CCC4)CC2)cc1 ; O=C(O)c1ccc(C2(NC(=O)c3cc4c(c5ccn(Cc6ccc(Cl)cc6)c35)CCC4)CC2)cc1 ; O=C(O)c1ccc(C2(NC(=O)c3cc4c(c5ccn(Cc6cccc(C(F)(F)F)c6)c35)CCC4)CC2)cc1 ; O=C(O)c1ccc(C2(NC(=O)c3cc4c(c5ccn(Cc6cccc(Cl)c6)c35)CCC4)CC2)cc1]

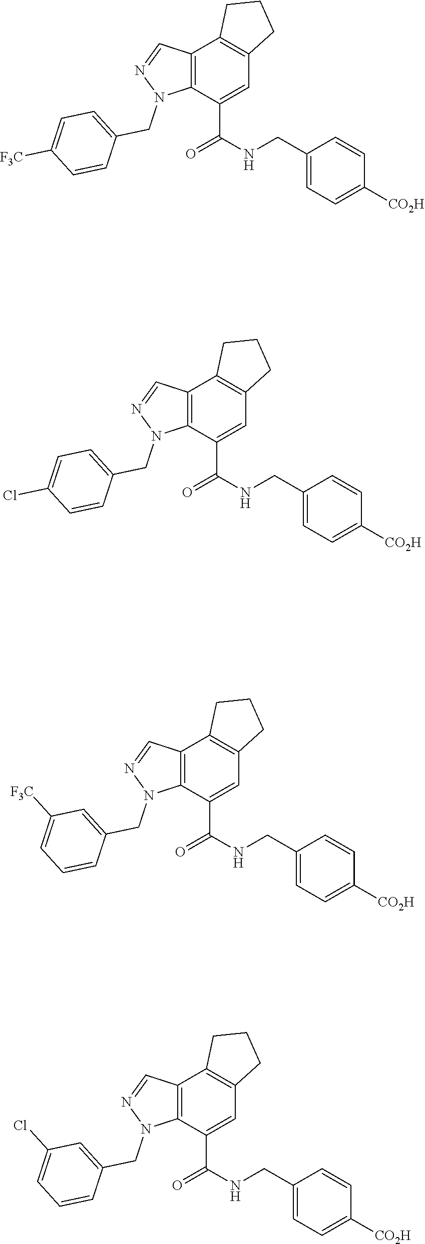 [SMILES: O=C(O)c1ccc(CNC(=O)c2cc3c(c4cnn(Cc5ccc(C(F)(F)F)cc5)c24)CCC3)cc1 ; O=C(O)c1ccc(CNC(=O)c2cc3c(c4cnn(Cc5ccc(Cl)cc5)c24)CCC3)cc1 ; O=C(O)c1ccc(CNC(=O)c2cc3c(c4cnn(Cc5cccc(C(F)(F)F)c5)c24)CCC3)cc1 ; O=C(O)c1ccc(CNC(=O)c2cc3c(c4cnn(Cc5cccc(Cl)c5)c24)CCC3)cc1]

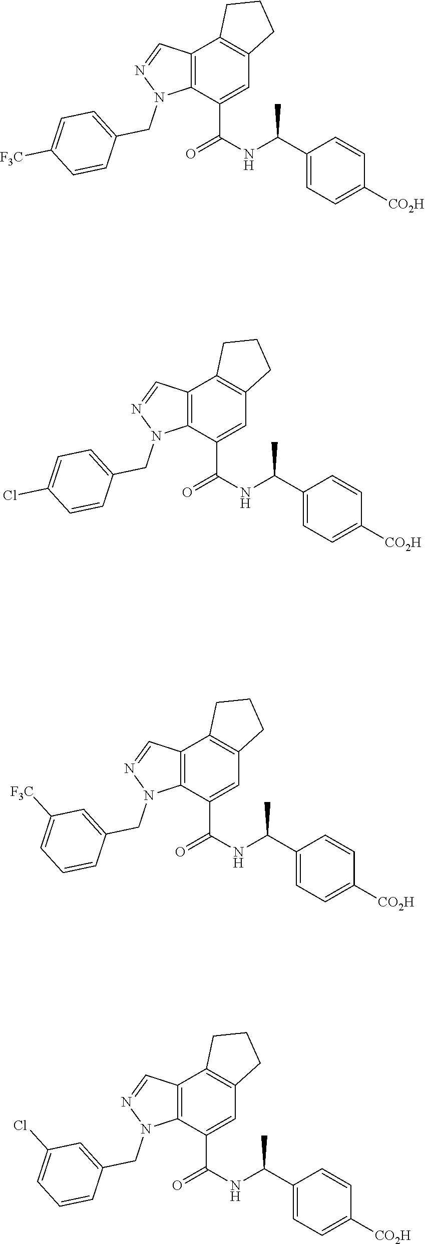 [SMILES: C[C@H](NC(=O)c1cc2c(c3cnn(Cc4ccc(C(F)(F)F)cc4)c13)CCC2)c1ccc(C(=O)O)cc1 ; C[C@H](NC(=O)c1cc2c(c3cnn(Cc4ccc(Cl)cc4)c13)CCC2)c1ccc(C(=O)O)cc1 ; C[C@H](NC(=O)c1cc2c(c3cnn(Cc4cccc(C(F)(F)F)c4)c13)CCC2)c1ccc(C(=O)O)cc1 ; C[C@H](NC(=O)c1cc2c(c3cnn(Cc4cccc(Cl)c4)c13)CCC2)c1ccc(C(=O)O)cc1]

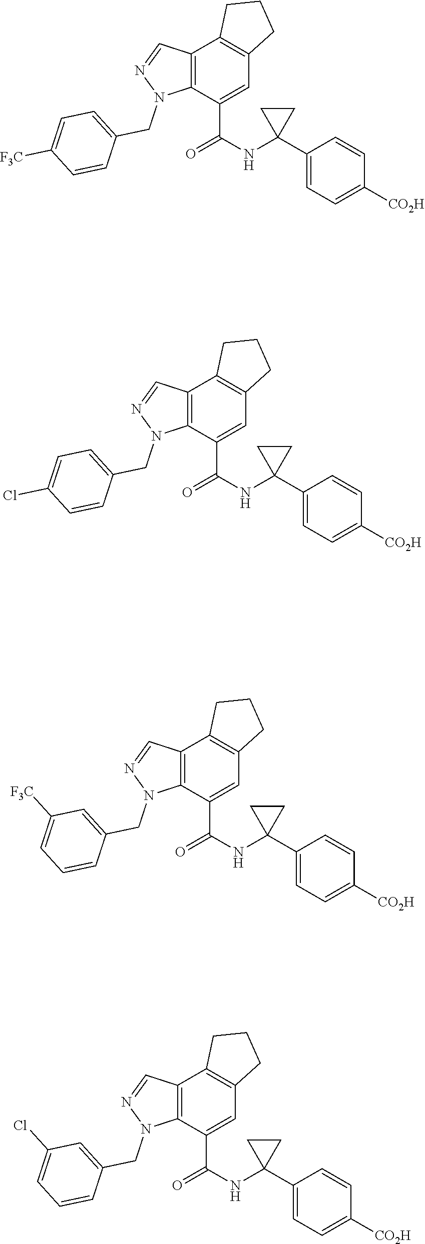 [SMILES: O=C(O)c1ccc(C2(NC(=O)c3cc4c(c5cnn(Cc6ccc(C(F)(F)F)cc6)c35)CCC4)CC2)cc1 ; O=C(O)c1ccc(C2(NC(=O)c3cc4c(c5cnn(Cc6ccc(Cl)cc6)c35)CCC4)CC2)cc1 ; O=C(O)c1ccc(C2(NC(=O)c3cc4c(c5cnn(Cc6cccc(C(F)(F)F)c6)c35)CCC4)CC2)cc1 ; O=C(O)c1ccc(C2(NC(=O)c3cc4c(c5cnn(Cc6cccc(Cl)c6)c35)CCC4)CC2)cc1]

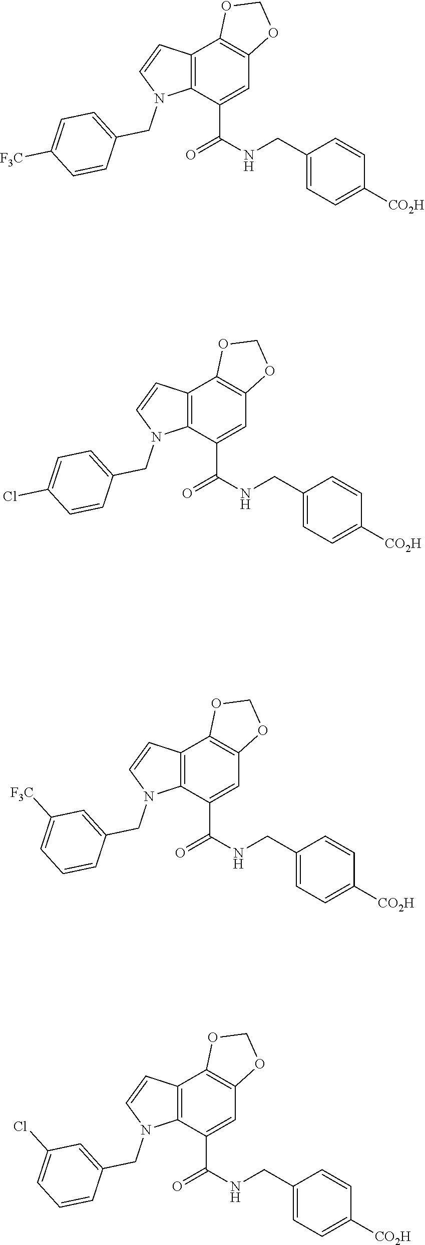 [SMILES: O=C(O)c1ccc(CNC(=O)c2cc3c(c4ccn(Cc5ccc(C(F)(F)F)cc5)c24)OCO3)cc1 ; O=C(O)c1ccc(CNC(=O)c2cc3c(c4ccn(Cc5ccc(Cl)cc5)c24)OCO3)cc1 ; O=C(O)c1ccc(CNC(=O)c2cc3c(c4ccn(Cc5cccc(C(F)(F)F)c5)c24)OCO3)cc1 ; O=C(O)c1ccc(CNC(=O)c2cc3c(c4ccn(Cc5cccc(Cl)c5)c24)OCO3)cc1]

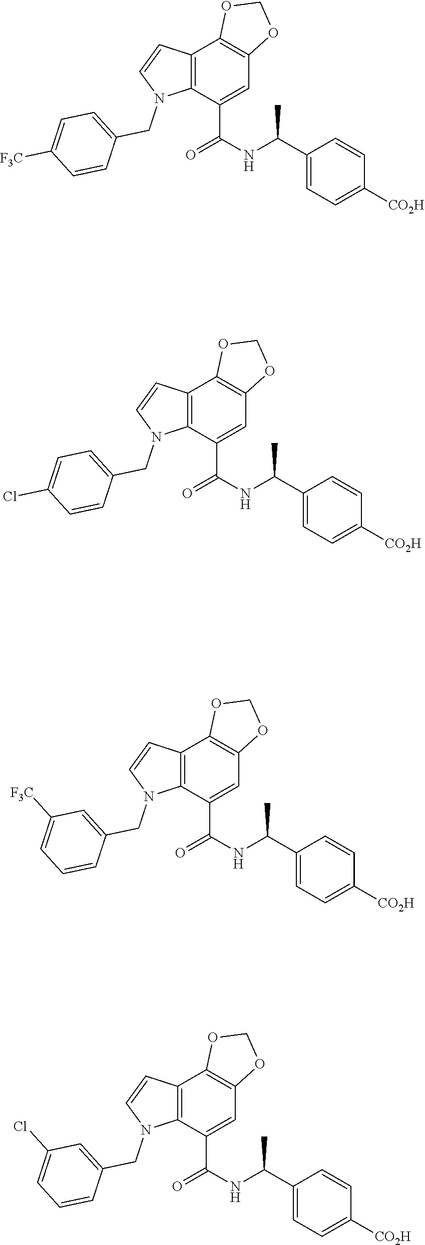 [SMILES: C[C@H](NC(=O)c1cc2c(c3ccn(Cc4ccc(C(F)(F)F)cc4)c13)OCO2)c1ccc(C(=O)O)cc1 ; C[C@H](NC(=O)c1cc2c(c3ccn(Cc4ccc(Cl)cc4)c13)OCO2)c1ccc(C(=O)O)cc1 ; C[C@H](NC(=O)c1cc2c(c3ccn(Cc4cccc(C(F)(F)F)c4)c13)OCO2)c1ccc(C(=O)O)cc1 ; C[C@H](NC(=O)c1cc2c(c3ccn(Cc4cccc(Cl)c4)c13)OCO2)c1ccc(C(=O)O)cc1]

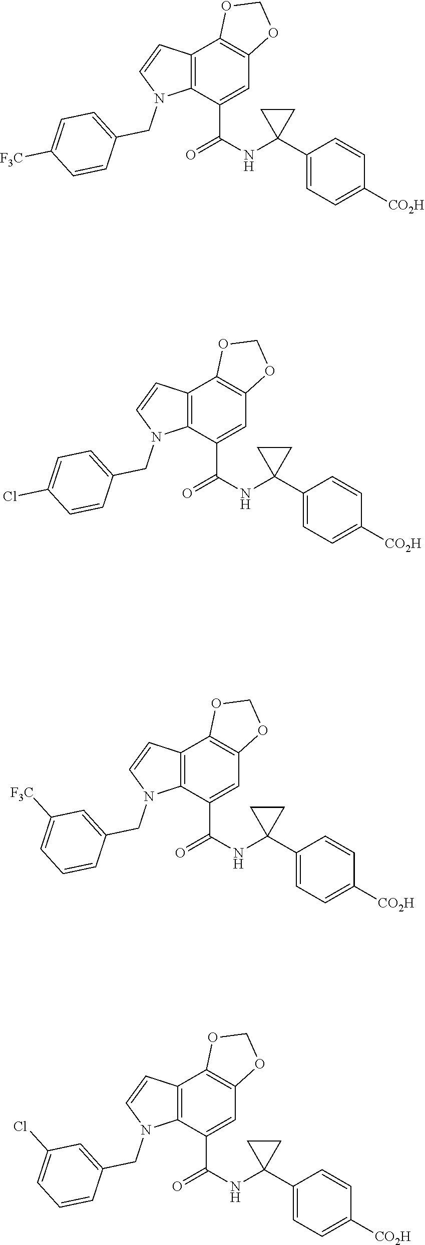 [SMILES: O=C(O)c1ccc(C2(NC(=O)c3cc4c(c5ccn(Cc6ccc(C(F)(F)F)cc6)c35)OCO4)CC2)cc1 ; O=C(O)c1ccc(C2(NC(=O)c3cc4c(c5ccn(Cc6ccc(Cl)cc6)c35)OCO4)CC2)cc1 ; O=C(O)c1ccc(C2(NC(=O)c3cc4c(c5ccn(Cc6cccc(C(F)(F)F)c6)c35)OCO4)CC2)cc1 ; O=C(O)c1ccc(C2(NC(=O)c3cc4c(c5ccn(Cc6cccc(Cl)c6)c35)OCO4)CC2)cc1]

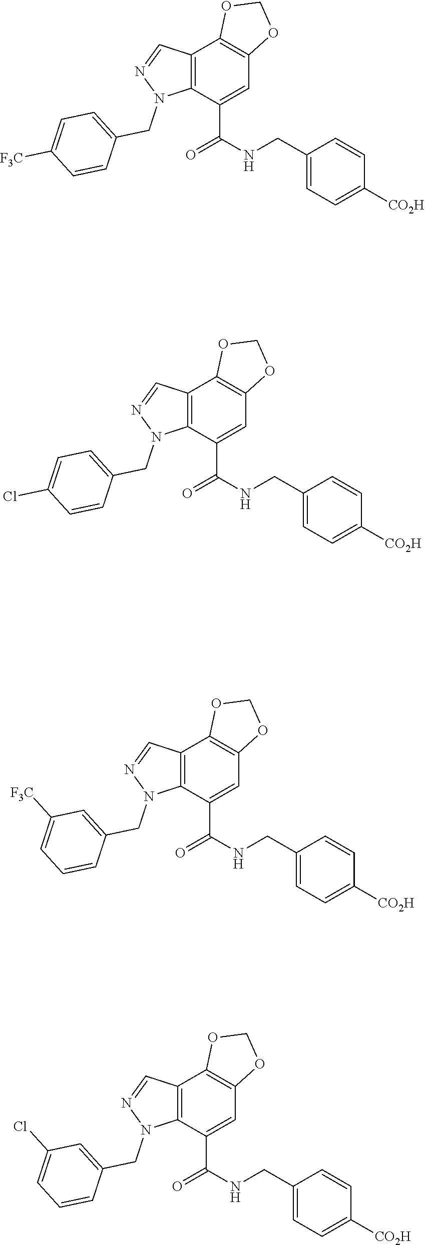 [SMILES: O=C(O)c1ccc(CNC(=O)c2cc3c(c4cnn(Cc5ccc(C(F)(F)F)cc5)c24)OCO3)cc1 ; O=C(O)c1ccc(CNC(=O)c2cc3c(c4cnn(Cc5ccc(Cl)cc5)c24)OCO3)cc1 ; O=C(O)c1ccc(CNC(=O)c2cc3c(c4cnn(Cc5cccc(C(F)(F)F)c5)c24)OCO3)cc1 ; O=C(O)c1ccc(CNC(=O)c2cc3c(c4cnn(Cc5cccc(Cl)c5)c24)OCO3)cc1]

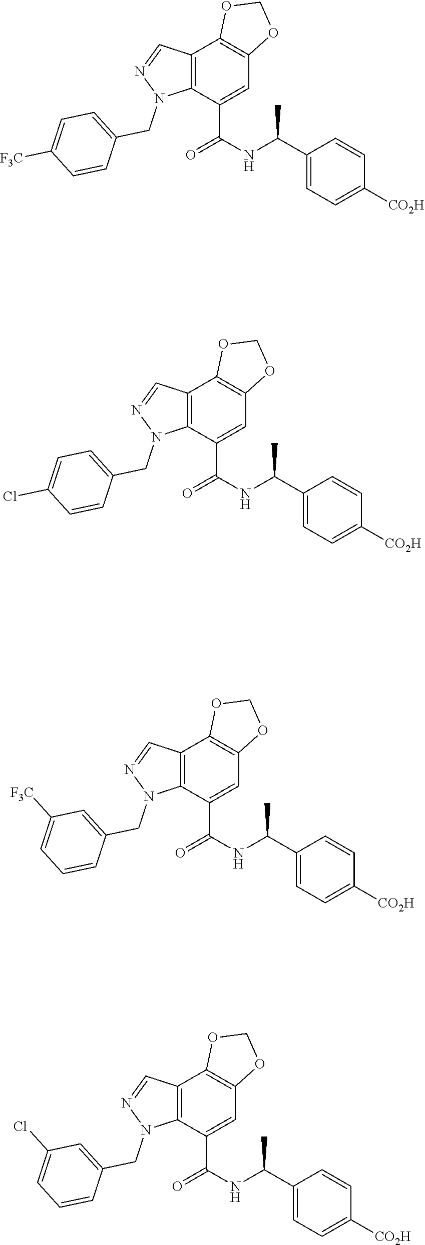 [SMILES: C[C@H](NC(=O)c1cc2c(c3cnn(Cc4ccc(C(F)(F)F)cc4)c13)OCO2)c1ccc(C(=O)O)cc1 ; C[C@H](NC(=O)c1cc2c(c3cnn(Cc4ccc(Cl)cc4)c13)OCO2)c1ccc(C(=O)O)cc1 ; C[C@H](NC(=O)c1cc2c(c3cnn(Cc4cccc(C(F)(F)F)c4)c13)OCO2)c1ccc(C(=O)O)cc1 ; C[C@H](NC(=O)c1cc2c(c3cnn(Cc4cccc(Cl)c4)c13)OCO2)c1ccc(C(=O)O)cc1]

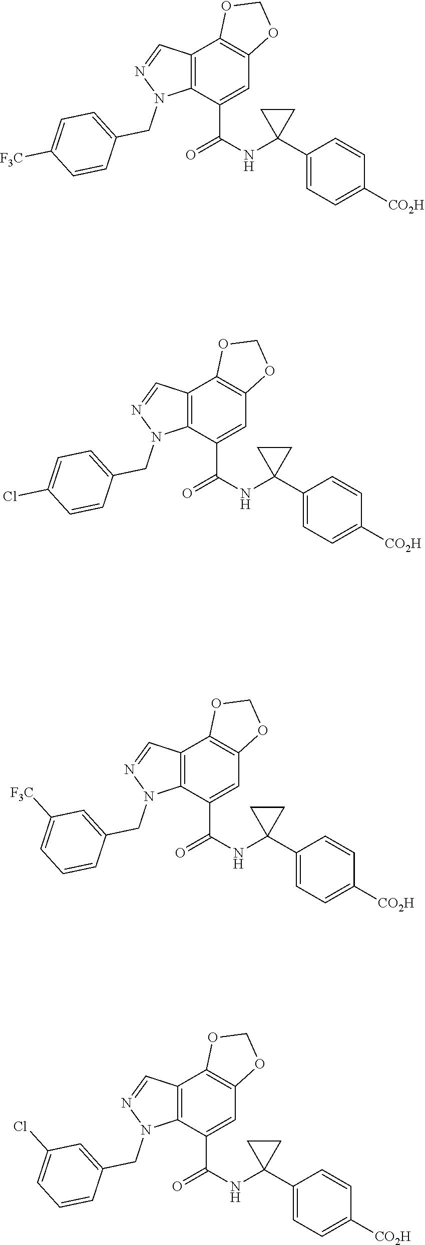 [SMILES: O=C(O)c1ccc(C2(NC(=O)c3cc4c(c5cnn(Cc6ccc(C(F)(F)F)cc6)c35)OCO4)CC2)cc1 ; O=C(O)c1ccc(C2(NC(=O)c3cc4c(c5cnn(Cc6ccc(Cl)cc6)c35)OCO4)CC2)cc1 ; O=C(O)c1ccc(C2(NC(=O)c3cc4c(c5cnn(Cc6cccc(C(F)(F)F)c6)c35)OCO4)CC2)cc1 ; O=C(O)c1ccc(C2(NC(=O)c3cc4c(c5cnn(Cc6cccc(Cl)c6)c35)OCO4)CC2)cc1]

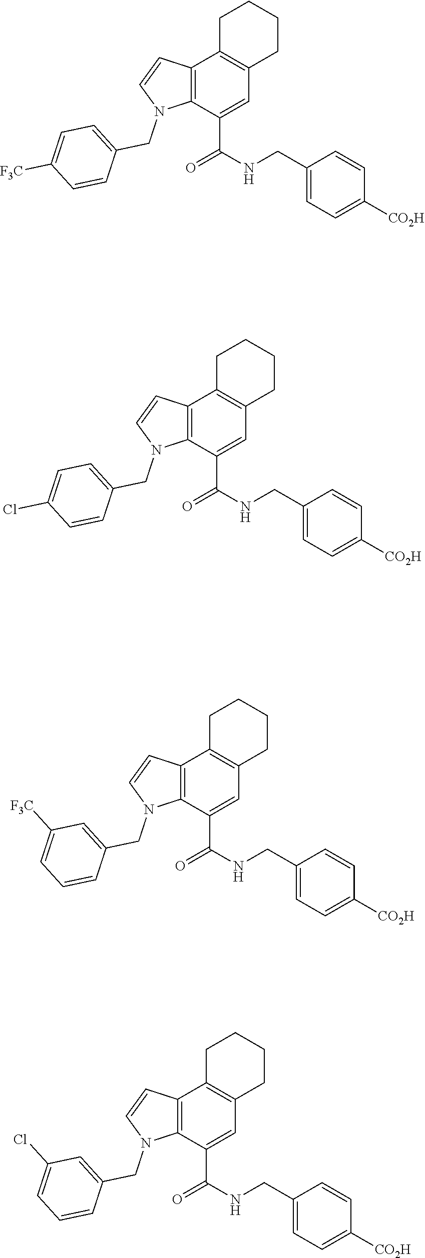 [SMILES: O=C(O)c1ccc(CNC(=O)c2cc3c(c4ccn(Cc5ccc(C(F)(F)F)cc5)c24)CCCC3)cc1 ; O=C(O)c1ccc(CNC(=O)c2cc3c(c4ccn(Cc5ccc(Cl)cc5)c24)CCCC3)cc1 ; O=C(O)c1ccc(CNC(=O)c2cc3c(c4ccn(Cc5cccc(C(F)(F)F)c5)c24)CCCC3)cc1 ; O=C(O)c1ccc(CNC(=O)c2cc3c(c4ccn(Cc5cccc(Cl)c5)c24)CCCC3)cc1]

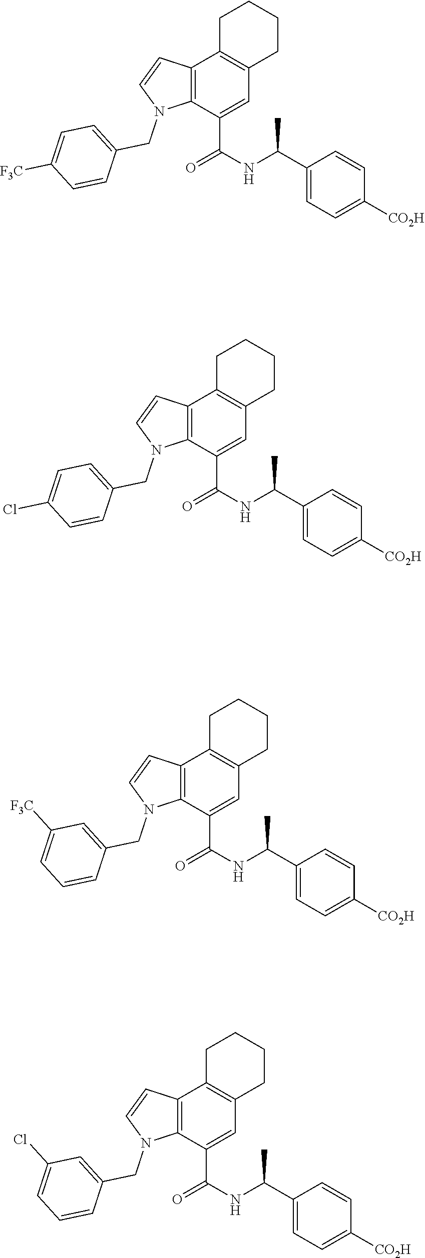 [SMILES: C[C@H](NC(=O)c1cc2c(c3ccn(Cc4ccc(C(F)(F)F)cc4)c13)CCCC2)c1ccc(C(=O)O)cc1 ; C[C@H](NC(=O)c1cc2c(c3ccn(Cc4ccc(Cl)cc4)c13)CCCC2)c1ccc(C(=O)O)cc1 ; C[C@H](NC(=O)c1cc2c(c3ccn(Cc4cccc(C(F)(F)F)c4)c13)CCCC2)c1ccc(C(=O)O)cc1 ; C[C@H](NC(=O)c1cc2c(c3ccn(Cc4cccc(Cl)c4)c13)CCCC2)c1ccc(C(=O)O)cc1]

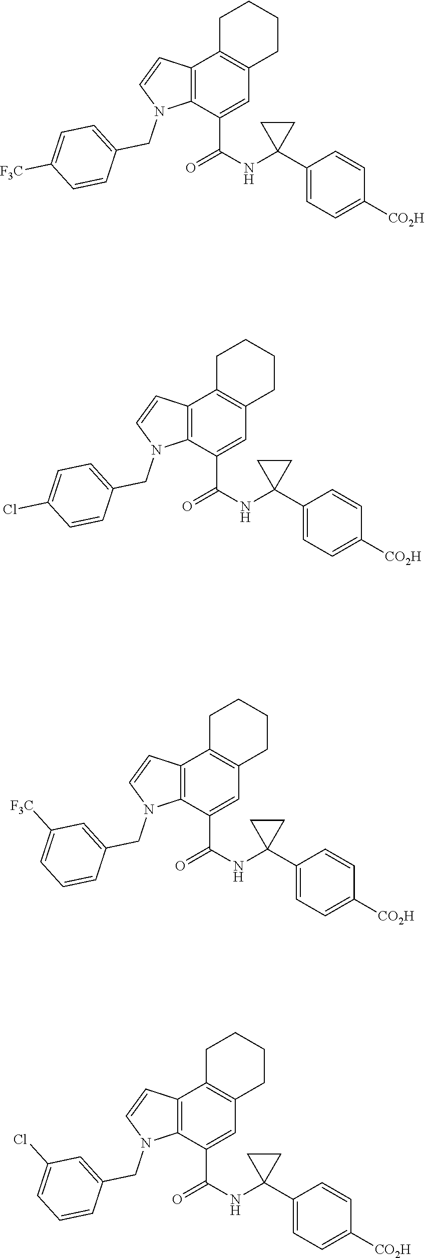 [SMILES: O=C(O)c1ccc(C2(NC(=O)c3cc4c(c5ccn(Cc6ccc(C(F)(F)F)cc6)c35)CCCC4)CC2)cc1 ; O=C(O)c1ccc(C2(NC(=O)c3cc4c(c5ccn(Cc6ccc(Cl)cc6)c35)CCCC4)CC2)cc1 ; O=C(O)c1ccc(C2(NC(=O)c3cc4c(c5ccn(Cc6cccc(C(F)(F)F)c6)c35)CCCC4)CC2)cc1 ; O=C(O)c1ccc(C2(NC(=O)c3cc4c(c5ccn(Cc6cccc(Cl)c6)c35)CCCC4)CC2)cc1]

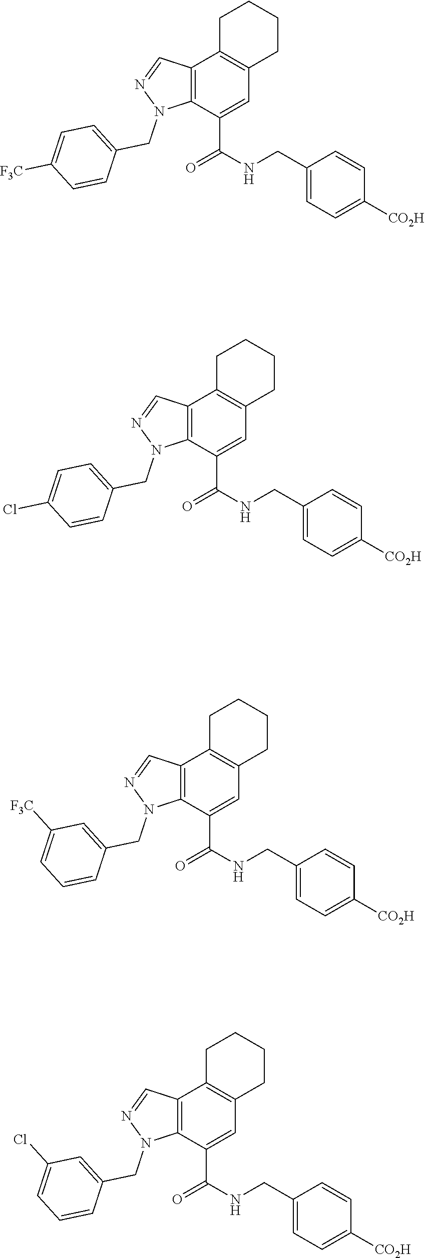 [SMILES: O=C(O)c1ccc(CNC(=O)c2cc3c(c4cnn(Cc5ccc(C(F)(F)F)cc5)c24)CCCC3)cc1 ; O=C(O)c1ccc(CNC(=O)c2cc3c(c4cnn(Cc5ccc(Cl)cc5)c24)CCCC3)cc1 ; O=C(O)c1ccc(CNC(=O)c2cc3c(c4cnn(Cc5cccc(C(F)(F)F)c5)c24)CCCC3)cc1 ; O=C(O)c1ccc(CNC(=O)c2cc3c(c4cnn(Cc5cccc(Cl)c5)c24)CCCC3)cc1]

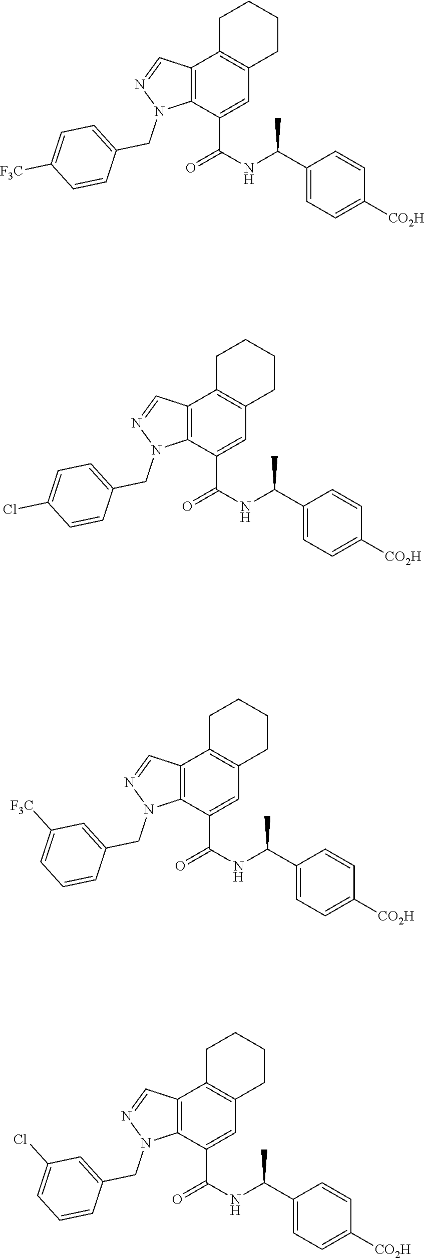 [SMILES: C[C@H](NC(=O)c1cc2c(c3cnn(Cc4ccc(C(F)(F)F)cc4)c13)CCCC2)c1ccc(C(=O)O)cc1 ; C[C@H](NC(=O)c1cc2c(c3cnn(Cc4ccc(Cl)cc4)c13)CCCC2)c1ccc(C(=O)O)cc1 ; C[C@H](NC(=O)c1cc2c(c3cnn(Cc4cccc(C(F)(F)F)c4)c13)CCCC2)c1ccc(C(=O)O)cc1 ; C[C@H](NC(=O)c1cc2c(c3cnn(Cc4cccc(Cl)c4)c13)CCCC2)c1ccc(C(=O)O)cc1]

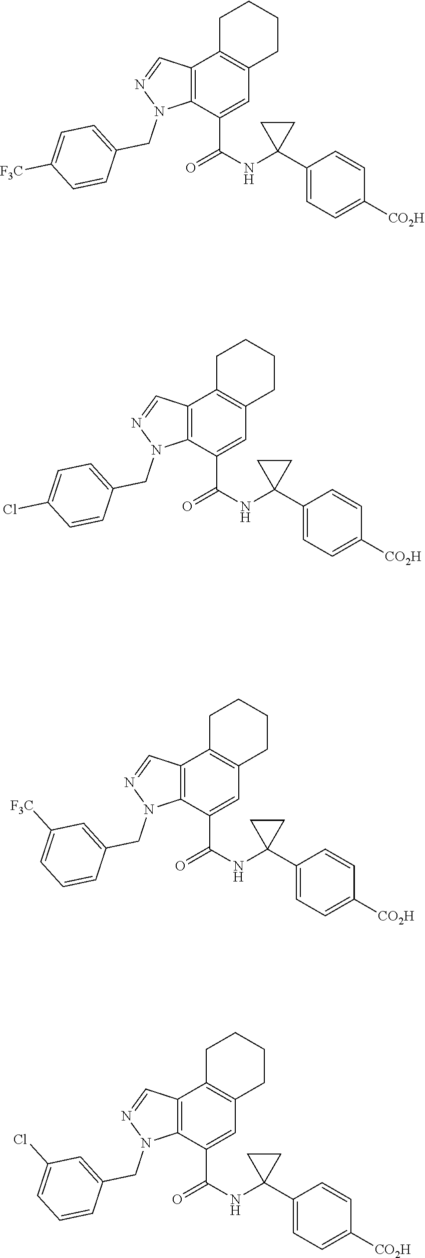 [SMILES: O=C(O)c1ccc(C2(NC(=O)c3cc4c(c5cnn(Cc6ccc(C(F)(F)F)cc6)c35)CCCC4)CC2)cc1 ; O=C(O)c1ccc(C2(NC(=O)c3cc4c(c5cnn(Cc6ccc(Cl)cc6)c35)CCCC4)CC2)cc1 ; O=C(O)c1ccc(C2(NC(=O)c3cc4c(c5cnn(Cc6cccc(C(F)(F)F)c6)c35)CCCC4)CC2)cc1 ; O=C(O)c1ccc(C2(NC(=O)c3cc4c(c5cnn(Cc6cccc(Cl)c6)c35)CCCC4)CC2)cc1]

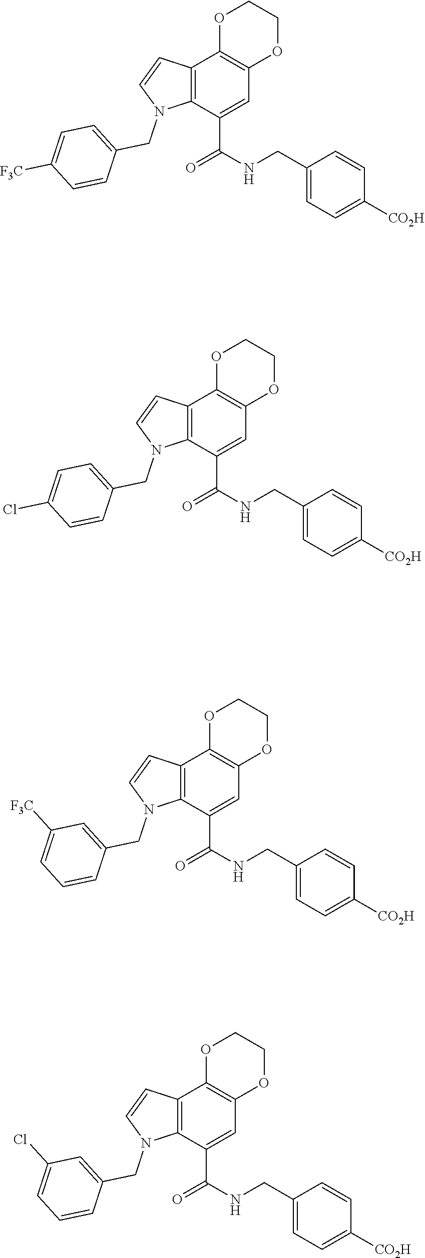 [SMILES: O=C(O)c1ccc(CNC(=O)c2cc3c(c4ccn(Cc5ccc(C(F)(F)F)cc5)c24)OCCO3)cc1 ; O=C(O)c1ccc(CNC(=O)c2cc3c(c4ccn(Cc5ccc(Cl)cc5)c24)OCCO3)cc1 ; O=C(O)c1ccc(CNC(=O)c2cc3c(c4ccn(Cc5cccc(C(F)(F)F)c5)c24)OCCO3)cc1 ; O=C(O)c1ccc(CNC(=O)c2cc3c(c4ccn(Cc5cccc(Cl)c5)c24)OCCO3)cc1]

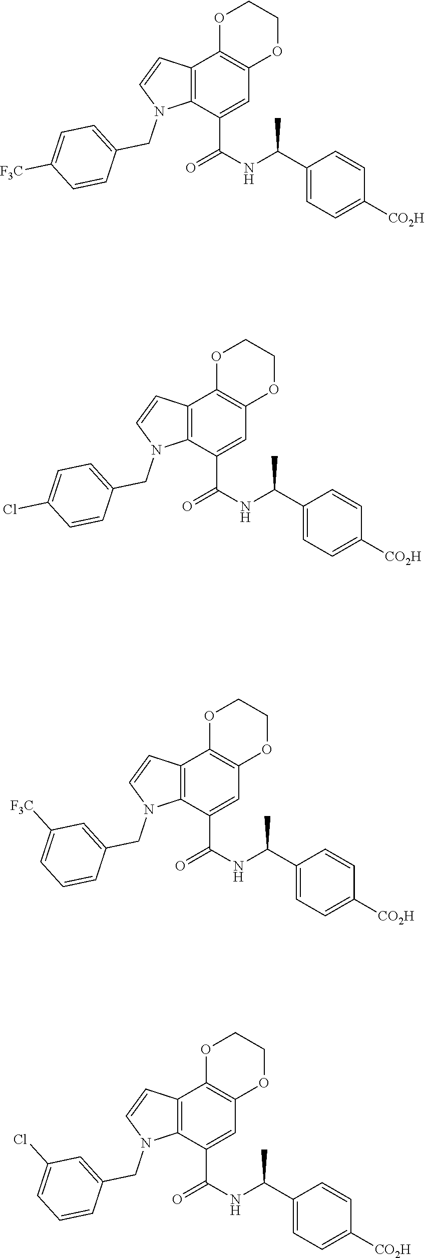 [SMILES: C[C@H](NC(=O)c1cc2c(c3ccn(Cc4ccc(C(F)(F)F)cc4)c13)OCCO2)c1ccc(C(=O)O)cc1 ; C[C@H](NC(=O)c1cc2c(c3ccn(Cc4ccc(Cl)cc4)c13)OCCO2)c1ccc(C(=O)O)cc1 ; C[C@H](NC(=O)c1cc2c(c3ccn(Cc4cccc(C(F)(F)F)c4)c13)OCCO2)c1ccc(C(=O)O)cc1 ; C[C@H](NC(=O)c1cc2c(c3ccn(Cc4cccc(Cl)c4)c13)OCCO2)c1ccc(C(=O)O)cc1]

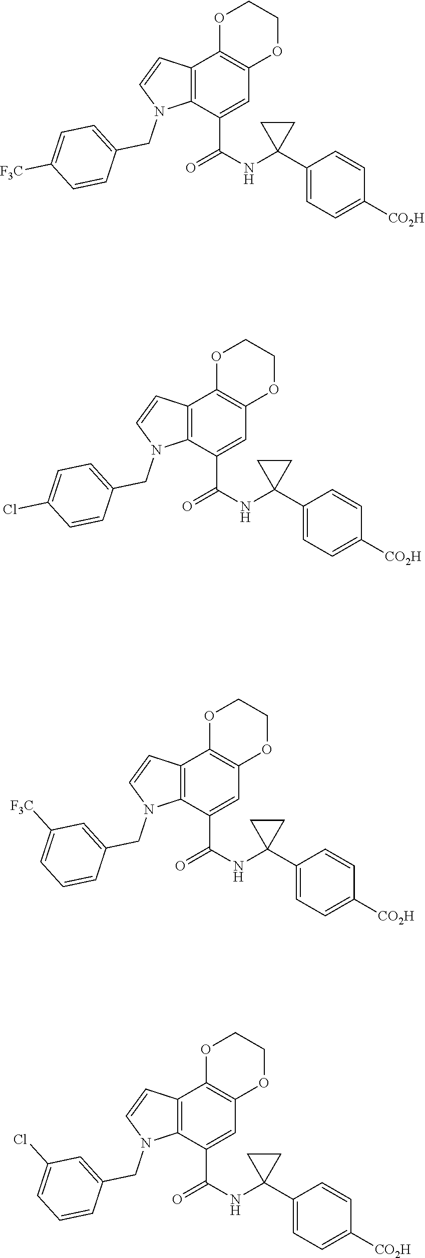 [SMILES: O=C(O)c1ccc(C2(NC(=O)c3cc4c(c5ccn(Cc6ccc(C(F)(F)F)cc6)c35)OCCO4)CC2)cc1 ; O=C(O)c1ccc(C2(NC(=O)c3cc4c(c5ccn(Cc6ccc(Cl)cc6)c35)OCCO4)CC2)cc1 ; O=C(O)c1ccc(C2(NC(=O)c3cc4c(c5ccn(Cc6cccc(C(F)(F)F)c6)c35)OCCO4)CC2)cc1 ; O=C(O)c1ccc(C2(NC(=O)c3cc4c(c5ccn(Cc6cccc(Cl)c6)c35)OCCO4)CC2)cc1]

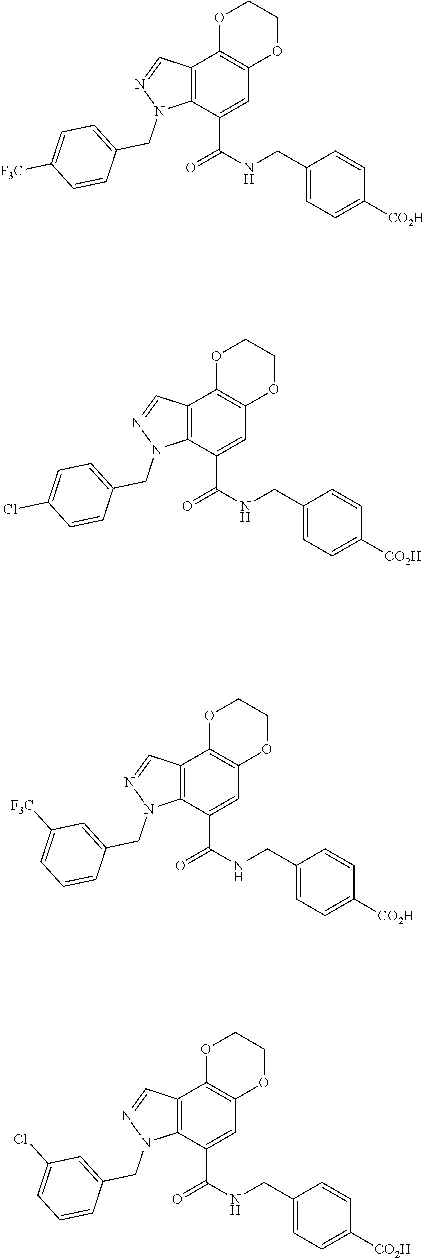 [SMILES: O=C(O)c1ccc(CNC(=O)c2cc3c(c4cnn(Cc5ccc(C(F)(F)F)cc5)c24)OCCO3)cc1 ; O=C(O)c1ccc(CNC(=O)c2cc3c(c4cnn(Cc5ccc(Cl)cc5)c24)OCCO3)cc1 ; O=C(O)c1ccc(CNC(=O)c2cc3c(c4cnn(Cc5cccc(C(F)(F)F)c5)c24)OCCO3)cc1 ; O=C(O)c1ccc(CNC(=O)c2cc3c(c4cnn(Cc5cccc(Cl)c5)c24)OCCO3)cc1]

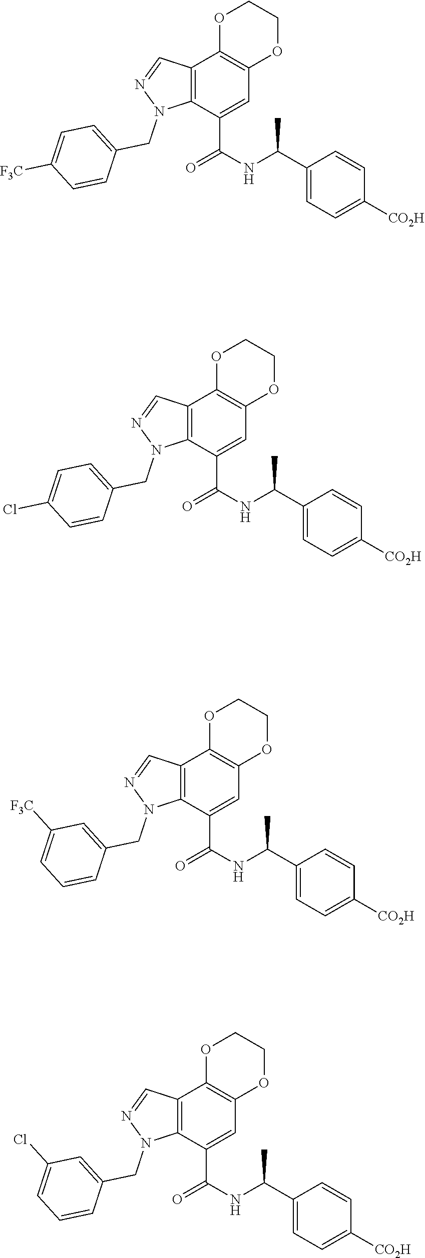 [SMILES: C[C@H](NC(=O)c1cc2c(c3cnn(Cc4ccc(C(F)(F)F)cc4)c13)OCCO2)c1ccc(C(=O)O)cc1 ; C[C@H](NC(=O)c1cc2c(c3cnn(Cc4ccc(Cl)cc4)c13)OCCO2)c1ccc(C(=O)O)cc1 ; C[C@H](NC(=O)c1cc2c(c3cnn(Cc4cccc(C(F)(F)F)c4)c13)OCCO2)c1ccc(C(=O)O)cc1 ; C[C@H](NC(=O)c1cc2c(c3cnn(Cc4cccc(Cl)c4)c13)OCCO2)c1ccc(C(=O)O)cc1]

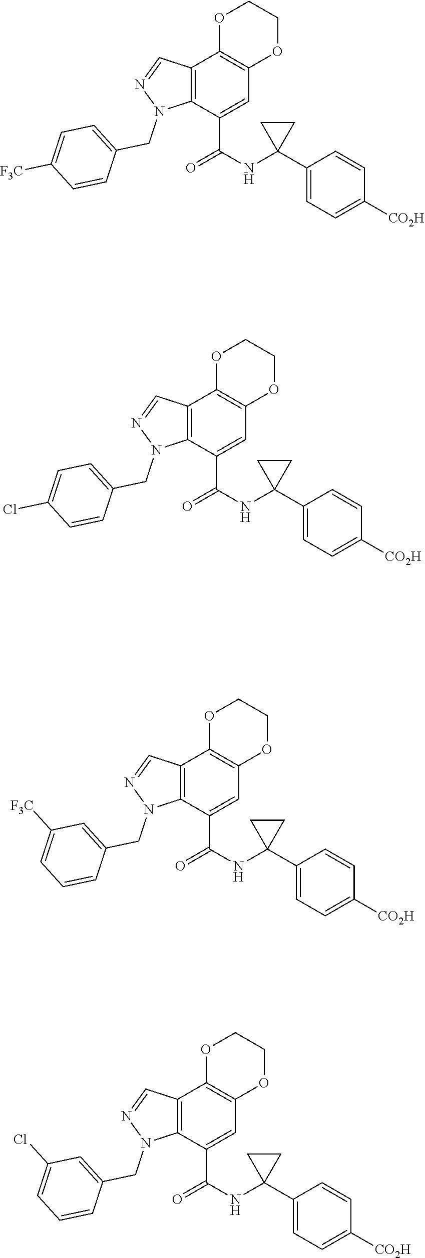 [SMILES: O=C(O)c1ccc(C2(NC(=O)c3cc4c(c5cnn(Cc6ccc(C(F)(F)F)cc6)c35)OCCO4)CC2)cc1 ; O=C(O)c1ccc(C2(NC(=O)c3cc4c(c5cnn(Cc6ccc(Cl)cc6)c35)OCCO4)CC2)cc1 ; O=C(O)c1ccc(C2(NC(=O)c3cc4c(c5cnn(Cc6cccc(C(F)(F)F)c6)c35)OCCO4)CC2)cc1 ; O=C(O)c1ccc(C2(NC(=O)c3cc4c(c5cnn(Cc6cccc(Cl)c6)c35)OCCO4)CC2)cc1]

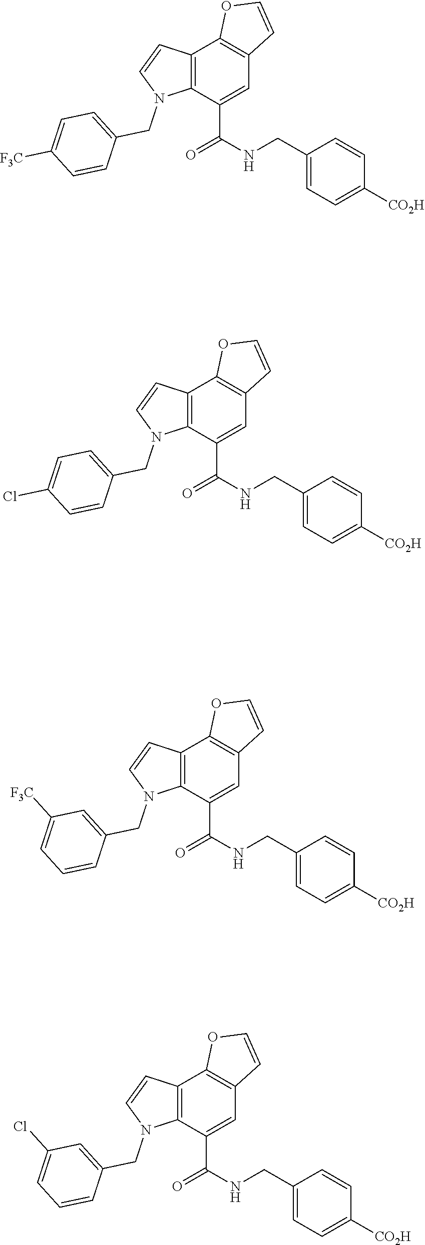 [SMILES: O=C(O)c1ccc(CNC(=O)c2cc3ccoc3c3ccn(Cc4ccc(C(F)(F)F)cc4)c23)cc1 ; O=C(O)c1ccc(CNC(=O)c2cc3ccoc3c3ccn(Cc4ccc(Cl)cc4)c23)cc1 ; O=C(O)c1ccc(CNC(=O)c2cc3ccoc3c3ccn(Cc4cccc(C(F)(F)F)c4)c23)cc1 ; O=C(O)c1ccc(CNC(=O)c2cc3ccoc3c3ccn(Cc4cccc(Cl)c4)c23)cc1]

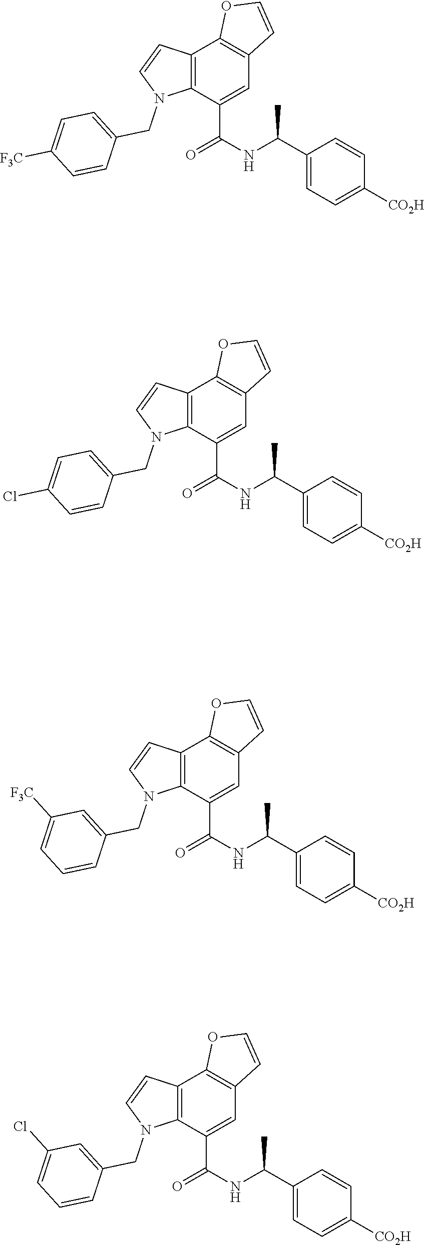 [SMILES: C[C@H](NC(=O)c1cc2ccoc2c2ccn(Cc3ccc(C(F)(F)F)cc3)c12)c1ccc(C(=O)O)cc1 ; C[C@H](NC(=O)c1cc2ccoc2c2ccn(Cc3ccc(Cl)cc3)c12)c1ccc(C(=O)O)cc1 ; C[C@H](NC(=O)c1cc2ccoc2c2ccn(Cc3cccc(C(F)(F)F)c3)c12)c1ccc(C(=O)O)cc1 ; C[C@H](NC(=O)c1cc2ccoc2c2ccn(Cc3cccc(Cl)c3)c12)c1ccc(C(=O)O)cc1]

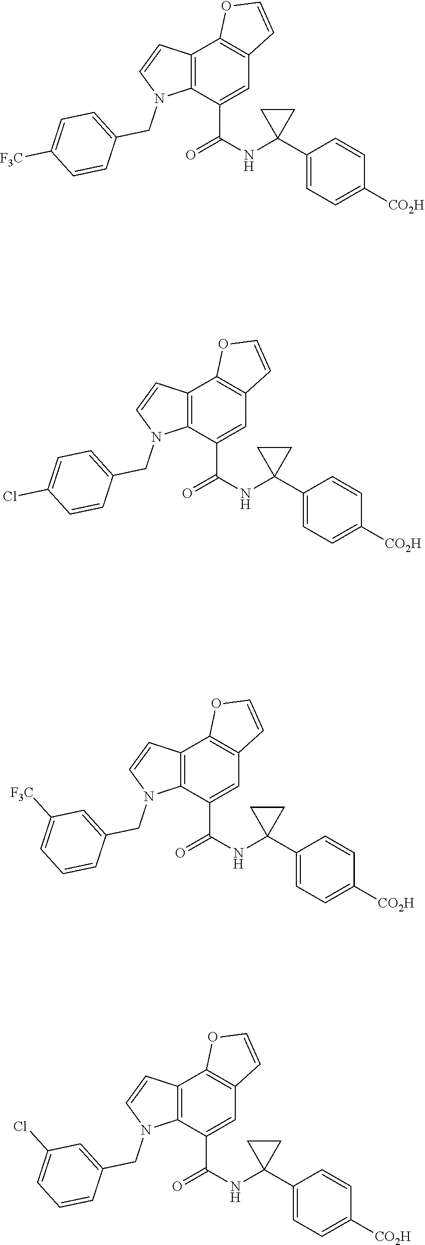 [SMILES: O=C(O)c1ccc(C2(NC(=O)c3cc4ccoc4c4ccn(Cc5ccc(C(F)(F)F)cc5)c34)CC2)cc1 ; O=C(O)c1ccc(C2(NC(=O)c3cc4ccoc4c4ccn(Cc5ccc(Cl)cc5)c34)CC2)cc1 ; O=C(O)c1ccc(C2(NC(=O)c3cc4ccoc4c4ccn(Cc5cccc(C(F)(F)F)c5)c34)CC2)cc1 ; O=C(O)c1ccc(C2(NC(=O)c3cc4ccoc4c4ccn(Cc5cccc(Cl)c5)c34)CC2)cc1]

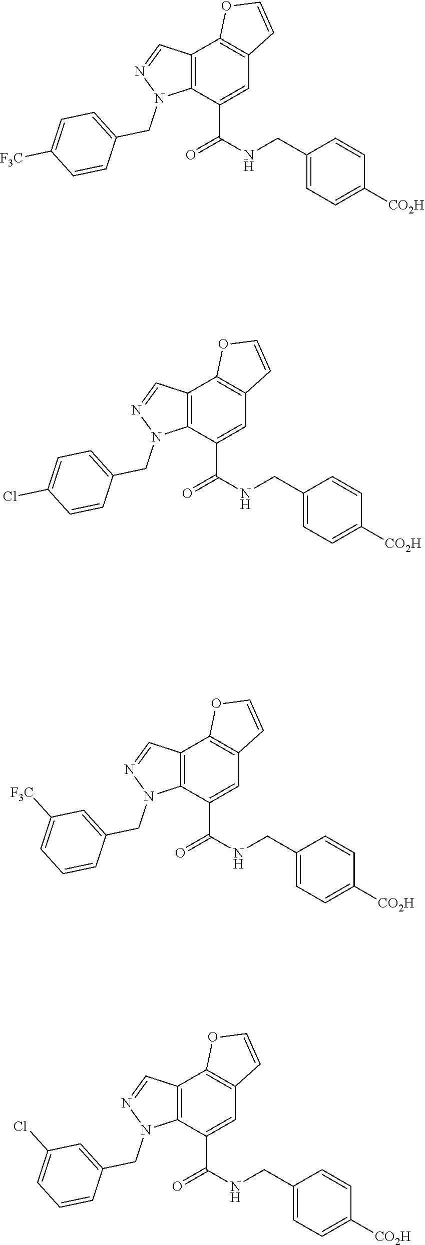 [SMILES: O=C(O)c1ccc(CNC(=O)c2cc3ccoc3c3cnn(Cc4ccc(C(F)(F)F)cc4)c23)cc1 ; O=C(O)c1ccc(CNC(=O)c2cc3ccoc3c3cnn(Cc4ccc(Cl)cc4)c23)cc1 ; O=C(O)c1ccc(CNC(=O)c2cc3ccoc3c3cnn(Cc4cccc(C(F)(F)F)c4)c23)cc1 ; O=C(O)c1ccc(CNC(=O)c2cc3ccoc3c3cnn(Cc4cccc(Cl)c4)c23)cc1]

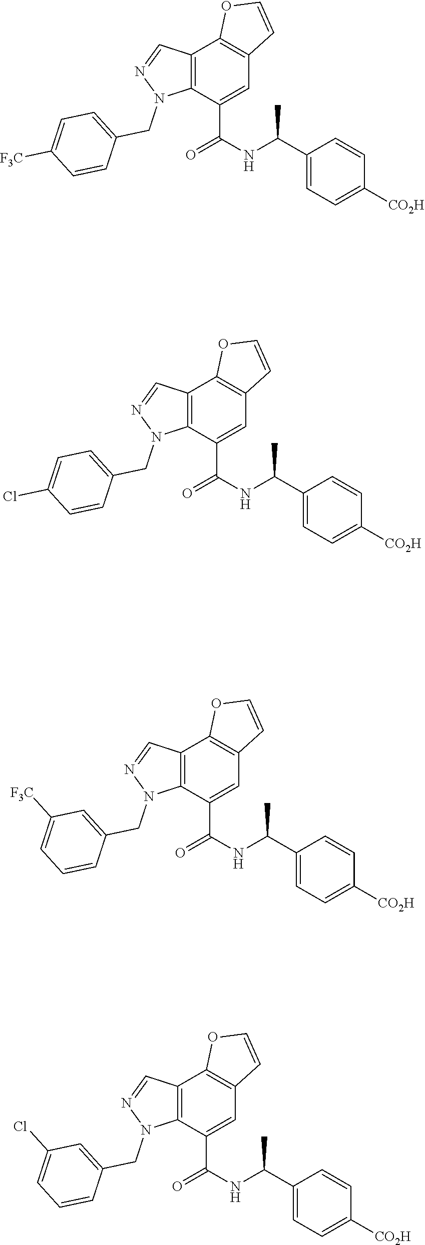 [SMILES: C[C@H](NC(=O)c1cc2ccoc2c2cnn(Cc3ccc(C(F)(F)F)cc3)c12)c1ccc(C(=O)O)cc1 ; C[C@H](NC(=O)c1cc2ccoc2c2cnn(Cc3ccc(Cl)cc3)c12)c1ccc(C(=O)O)cc1 ; C[C@H](NC(=O)c1cc2ccoc2c2cnn(Cc3cccc(C(F)(F)F)c3)c12)c1ccc(C(=O)O)cc1 ; C[C@H](NC(=O)c1cc2ccoc2c2cnn(Cc3cccc(Cl)c3)c12)c1ccc(C(=O)O)cc1]

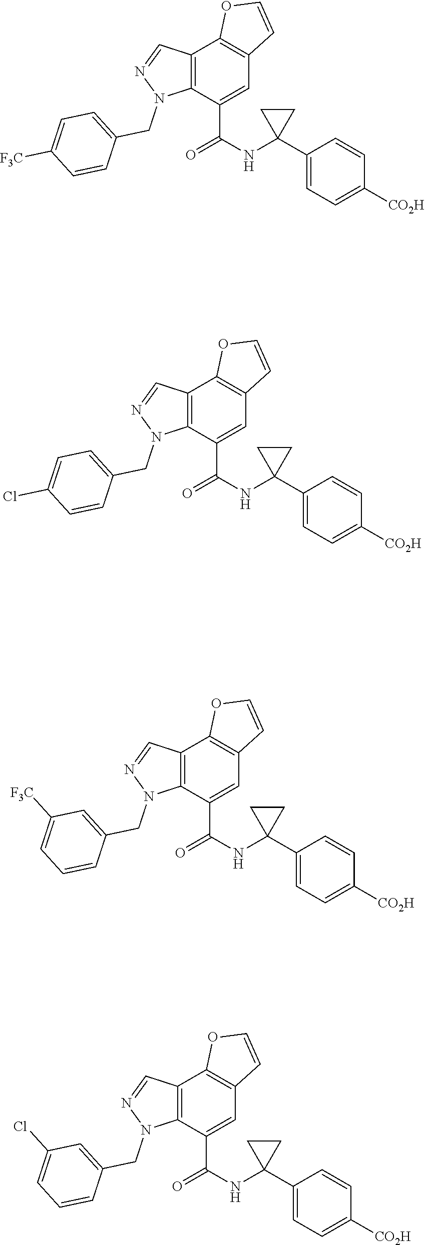 [SMILES: O=C(O)c1ccc(C2(NC(=O)c3cc4ccoc4c4cnn(Cc5ccc(C(F)(F)F)cc5)c34)CC2)cc1 ; O=C(O)c1ccc(C2(NC(=O)c3cc4ccoc4c4cnn(Cc5ccc(Cl)cc5)c34)CC2)cc1 ; O=C(O)c1ccc(C2(NC(=O)c3cc4ccoc4c4cnn(Cc5cccc(C(F)(F)F)c5)c34)CC2)cc1 ; O=C(O)c1ccc(C2(NC(=O)c3cc4ccoc4c4cnn(Cc5cccc(Cl)c5)c34)CC2)cc1]

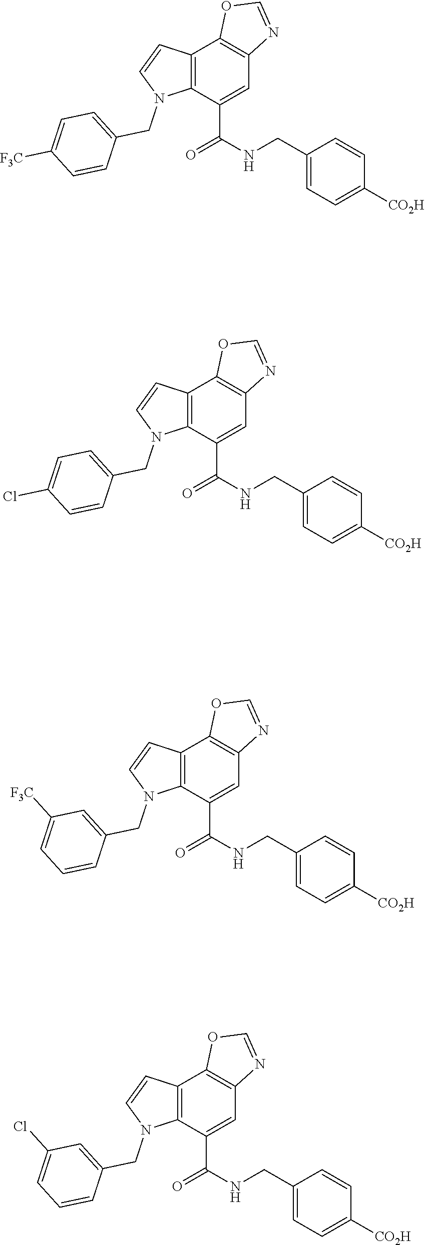 [SMILES: O=C(O)c1ccc(CNC(=O)c2cc3ncoc3c3ccn(Cc4ccc(C(F)(F)F)cc4)c23)cc1 ; O=C(O)c1ccc(CNC(=O)c2cc3ncoc3c3ccn(Cc4ccc(Cl)cc4)c23)cc1 ; O=C(O)c1ccc(CNC(=O)c2cc3ncoc3c3ccn(Cc4cccc(C(F)(F)F)c4)c23)cc1 ; O=C(O)c1ccc(CNC(=O)c2cc3ncoc3c3ccn(Cc4cccc(Cl)c4)c23)cc1]

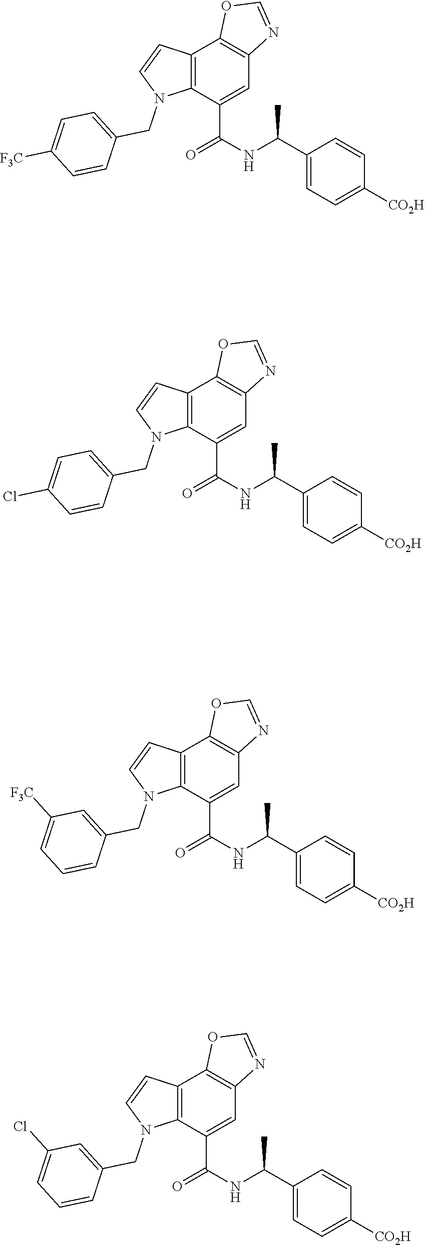 [SMILES: C[C@H](NC(=O)c1cc2ncoc2c2ccn(Cc3ccc(C(F)(F)F)cc3)c12)c1ccc(C(=O)O)cc1 ; C[C@H](NC(=O)c1cc2ncoc2c2ccn(Cc3ccc(Cl)cc3)c12)c1ccc(C(=O)O)cc1 ; C[C@H](NC(=O)c1cc2ncoc2c2ccn(Cc3cccc(C(F)(F)F)c3)c12)c1ccc(C(=O)O)cc1 ; C[C@H](NC(=O)c1cc2ncoc2c2ccn(Cc3cccc(Cl)c3)c12)c1ccc(C(=O)O)cc1]

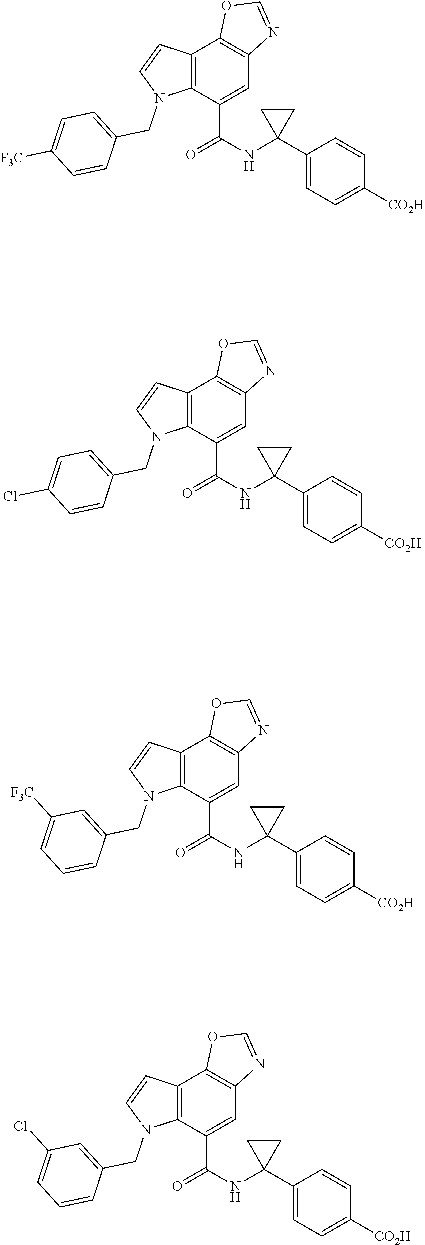 [SMILES: O=C(O)c1ccc(C2(NC(=O)c3cc4ncoc4c4ccn(Cc5ccc(C(F)(F)F)cc5)c34)CC2)cc1 ; O=C(O)c1ccc(C2(NC(=O)c3cc4ncoc4c4ccn(Cc5ccc(Cl)cc5)c34)CC2)cc1 ; O=C(O)c1ccc(C2(NC(=O)c3cc4ncoc4c4ccn(Cc5cccc(C(F)(F)F)c5)c34)CC2)cc1 ; O=C(O)c1ccc(C2(NC(=O)c3cc4ncoc4c4ccn(Cc5cccc(Cl)c5)c34)CC2)cc1]

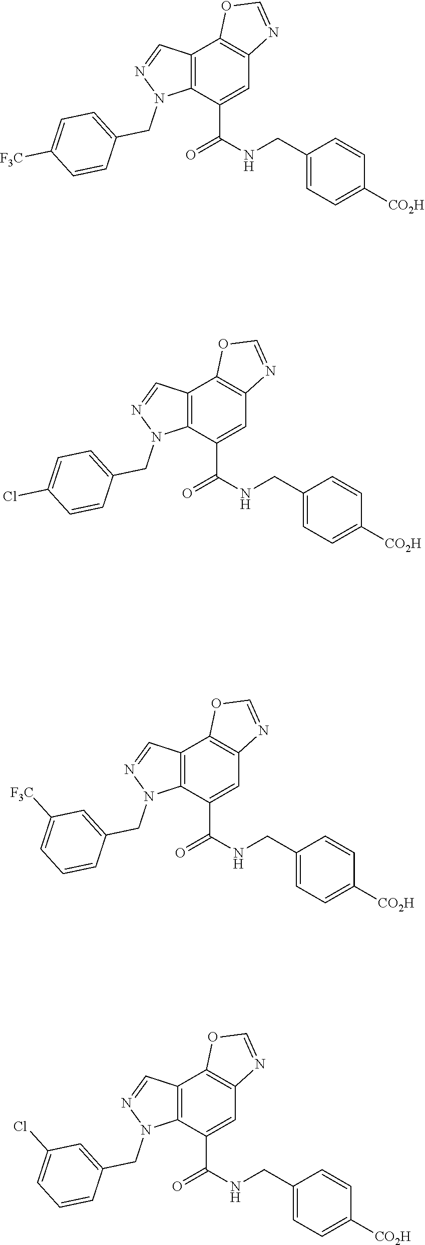 [SMILES: O=C(O)c1ccc(CNC(=O)c2cc3ncoc3c3cnn(Cc4ccc(C(F)(F)F)cc4)c23)cc1 ; O=C(O)c1ccc(CNC(=O)c2cc3ncoc3c3cnn(Cc4ccc(Cl)cc4)c23)cc1 ; O=C(O)c1ccc(CNC(=O)c2cc3ncoc3c3cnn(Cc4cccc(C(F)(F)F)c4)c23)cc1 ; O=C(O)c1ccc(CNC(=O)c2cc3ncoc3c3cnn(Cc4cccc(Cl)c4)c23)cc1]

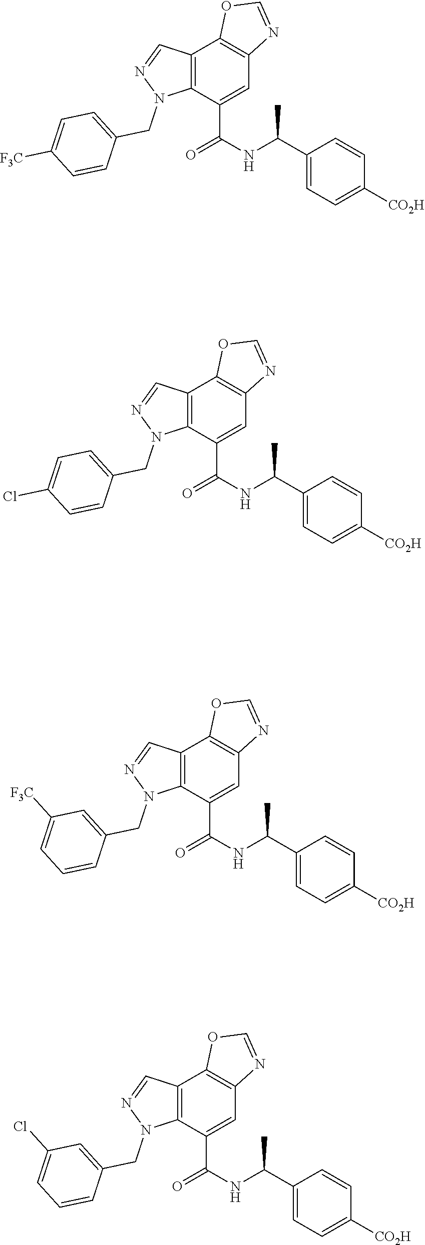 [SMILES: C[C@H](NC(=O)c1cc2ncoc2c2cnn(Cc3ccc(C(F)(F)F)cc3)c12)c1ccc(C(=O)O)cc1 ; C[C@H](NC(=O)c1cc2ncoc2c2cnn(Cc3ccc(Cl)cc3)c12)c1ccc(C(=O)O)cc1 ; C[C@H](NC(=O)c1cc2ncoc2c2cnn(Cc3cccc(C(F)(F)F)c3)c12)c1ccc(C(=O)O)cc1 ; C[C@H](NC(=O)c1cc2ncoc2c2cnn(Cc3cccc(Cl)c3)c12)c1ccc(C(=O)O)cc1]

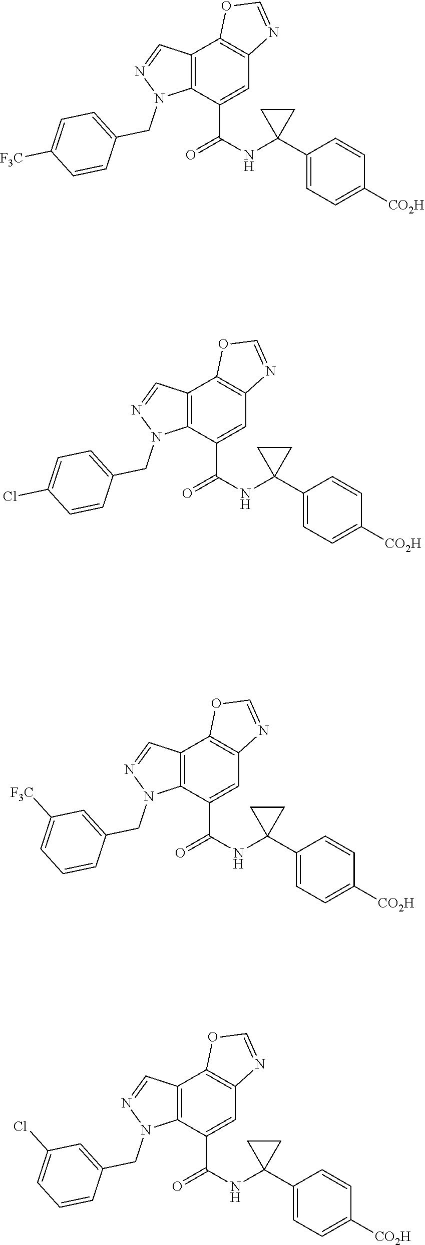 [SMILES: O=C(O)c1ccc(C2(NC(=O)c3cc4ncoc4c4cnn(Cc5ccc(C(F)(F)F)cc5)c34)CC2)cc1 ; O=C(O)c1ccc(C2(NC(=O)c3cc4ncoc4c4cnn(Cc5ccc(Cl)cc5)c34)CC2)cc1 ; O=C(O)c1ccc(C2(NC(=O)c3cc4ncoc4c4cnn(Cc5cccc(C(F)(F)F)c5)c34)CC2)cc1 ; O=C(O)c1ccc(C2(NC(=O)c3cc4ncoc4c4cnn(Cc5cccc(Cl)c5)c34)CC2)cc1]

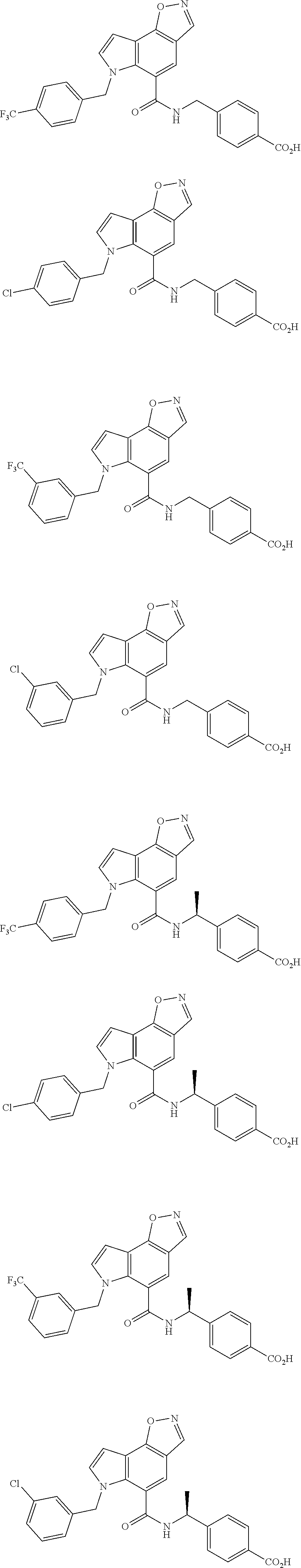

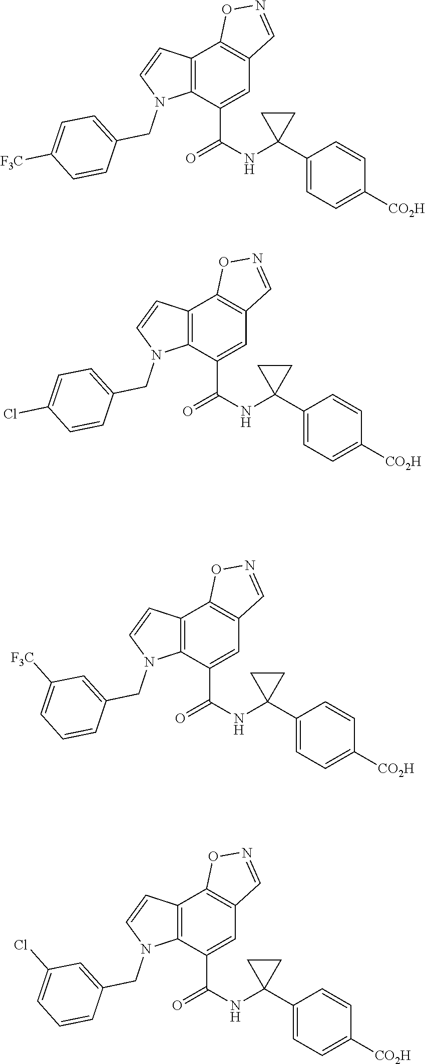 [SMILES: O=C(O)c1ccc(C2(NC(=O)c3cc4cnoc4c4ccn(Cc5ccc(C(F)(F)F)cc5)c34)CC2)cc1 ; O=C(O)c1ccc(C2(NC(=O)c3cc4cnoc4c4ccn(Cc5ccc(Cl)cc5)c34)CC2)cc1 ; O=C(O)c1ccc(C2(NC(=O)c3cc4cnoc4c4ccn(Cc5cccc(C(F)(F)F)c5)c34)CC2)cc1 ; O=C(O)c1ccc(C2(NC(=O)c3cc4cnoc4c4ccn(Cc5cccc(Cl)c5)c34)CC2)cc1]

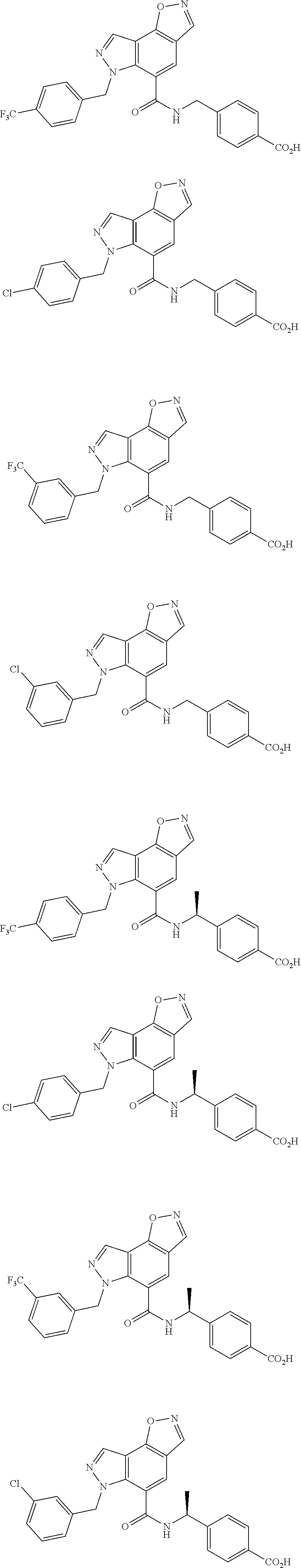

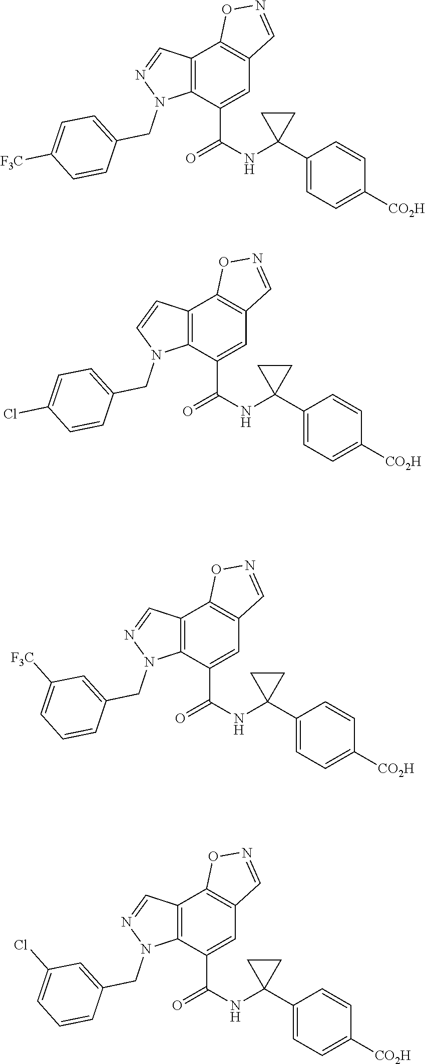 [SMILES: O=C(O)c1ccc(C2(NC(=O)c3cc4cnoc4c4cnn(Cc5ccc(C(F)(F)F)cc5)c34)CC2)cc1 ; O=C(O)c1ccc(C2(NC(=O)c3cc4cnoc4c4ccn(Cc5ccc(Cl)cc5)c34)CC2)cc1 ; O=C(O)c1ccc(C2(NC(=O)c3cc4cnoc4c4cnn(Cc5cccc(C(F)(F)F)c5)c34)CC2)cc1 ; O=C(O)c1ccc(C2(NC(=O)c3cc4cnoc4c4cnn(Cc5cccc(Cl)c5)c34)CC2)cc1]

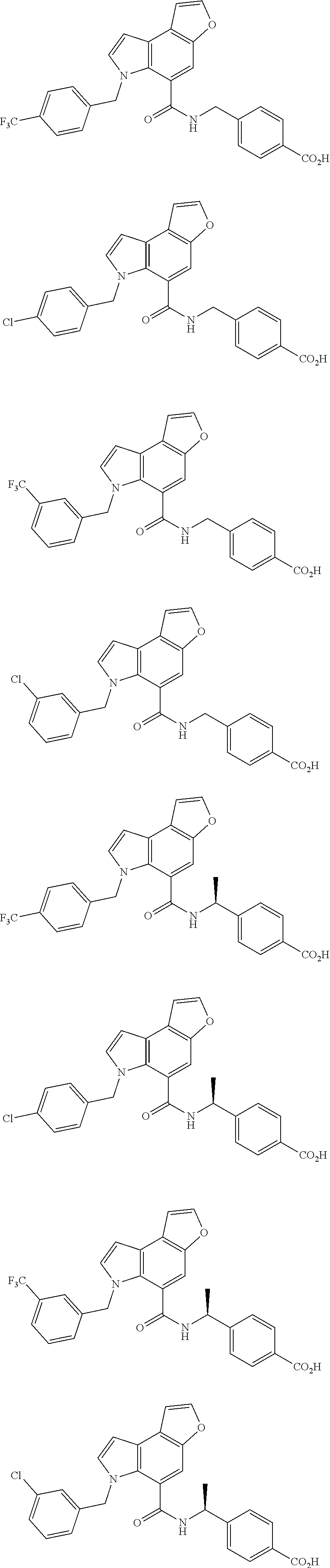

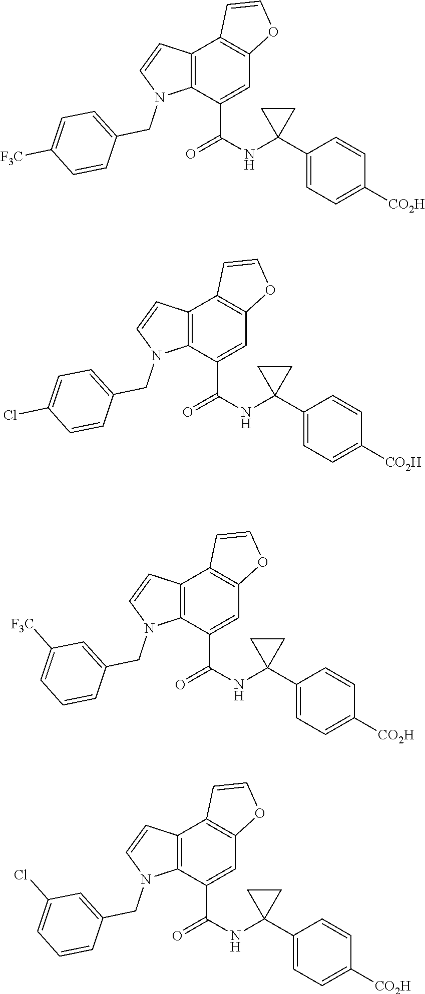 [SMILES: O=C(O)c1ccc(C2(NC(=O)c3cc4occc4c4ccn(Cc5ccc(C(F)(F)F)cc5)c34)CC2)cc1 ; O=C(O)c1ccc(C2(NC(=O)c3cc4occc4c4ccn(Cc5ccc(Cl)cc5)c34)CC2)cc1 ; O=C(O)c1ccc(C2(NC(=O)c3cc4occc4c4ccn(Cc5cccc(C(F)(F)F)c5)c34)CC2)cc1 ; O=C(O)c1ccc(C2(NC(=O)c3cc4occc4c4ccn(Cc5cccc(Cl)c5)c34)CC2)cc1]

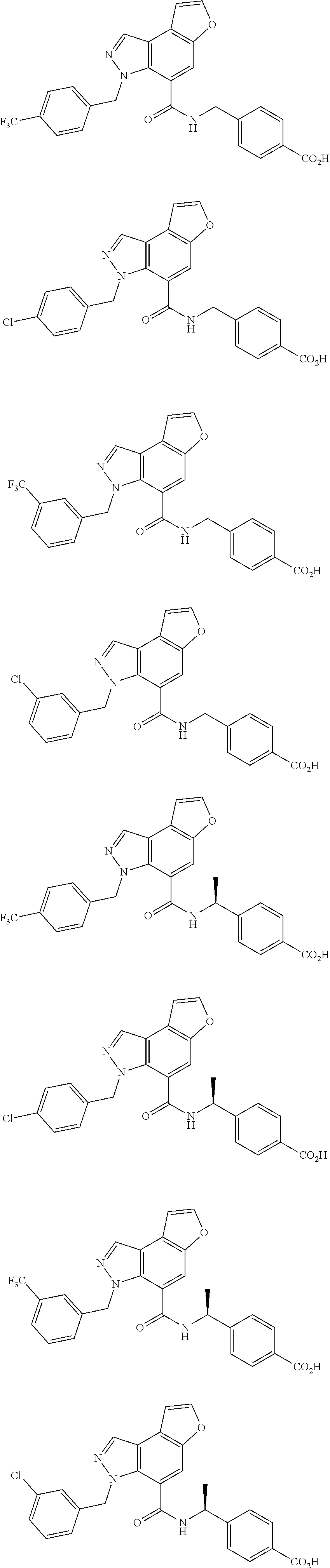

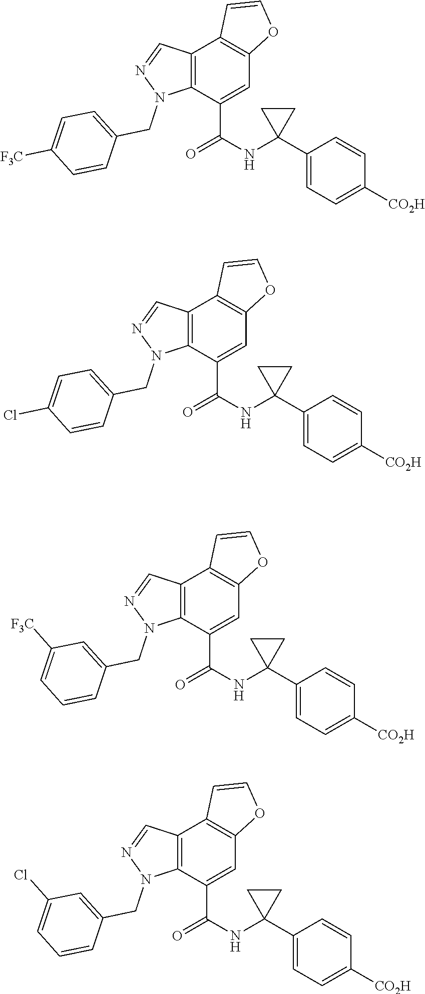 [SMILES: O=C(O)c1ccc(C2(NC(=O)c3cc4occc4c4cnn(Cc5ccc(C(F)(F)F)cc5)c34)CC2)cc1 ; O=C(O)c1ccc(C2(NC(=O)c3cc4occc4c4cnn(Cc5ccc(Cl)cc5)c34)CC2)cc1 ; O=C(O)c1ccc(C2(NC(=O)c3cc4occc4c4cnn(Cc5cccc(C(F)(F)F)c5)c34)CC2)cc1 ; O=C(O)c1ccc(C2(NC(=O)c3cc4occc4c4cnn(Cc5cccc(Cl)c5)c34)CC2)cc1]

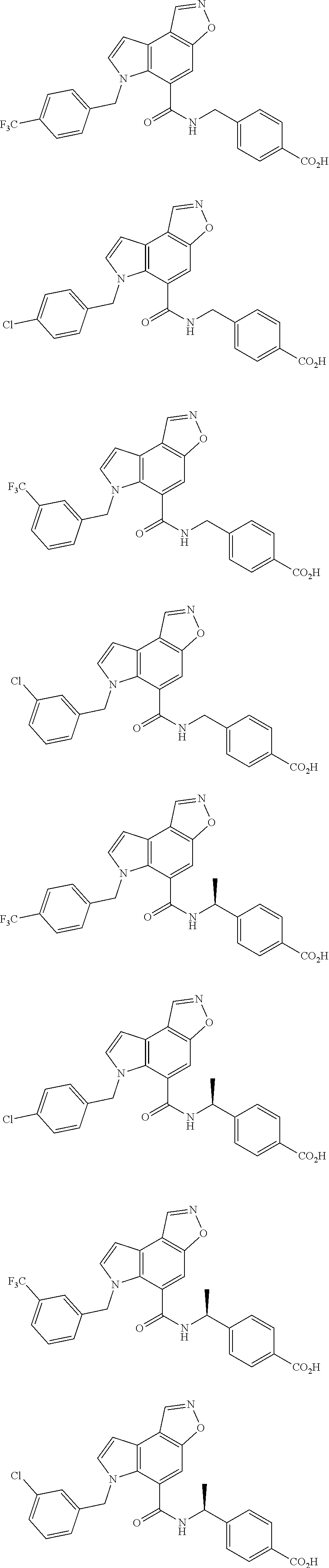

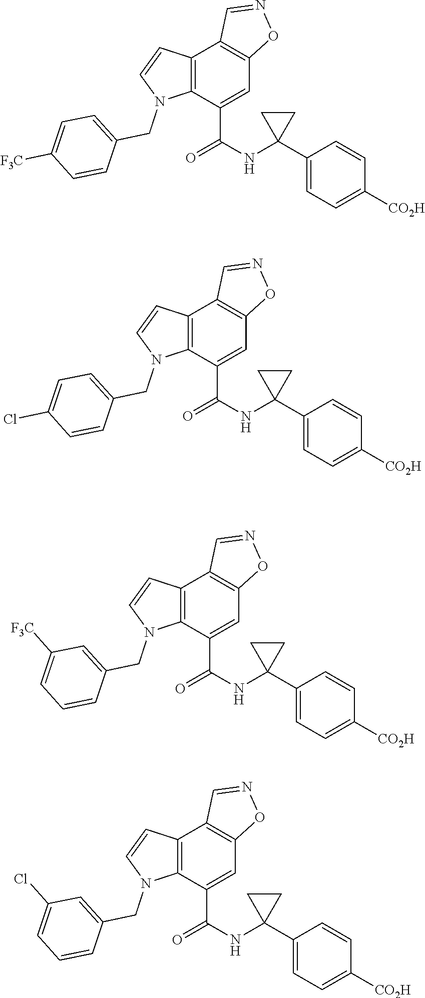 [SMILES: O=C(O)c1ccc(C2(NC(=O)c3cc4oncc4c4ccn(Cc5ccc(C(F)(F)F)cc5)c34)CC2)cc1 ; O=C(O)c1ccc(C2(NC(=O)c3cc4oncc4c4ccn(Cc5ccc(Cl)cc5)c34)CC2)cc1 ; O=C(O)c1ccc(C2(NC(=O)c3cc4oncc4c4ccn(Cc5cccc(C(F)(F)F)c5)c34)CC2)cc1 ; O=C(O)c1ccc(C2(NC(=O)c3cc4oncc4c4ccn(Cc5cccc(Cl)c5)c34)CC2)cc1]

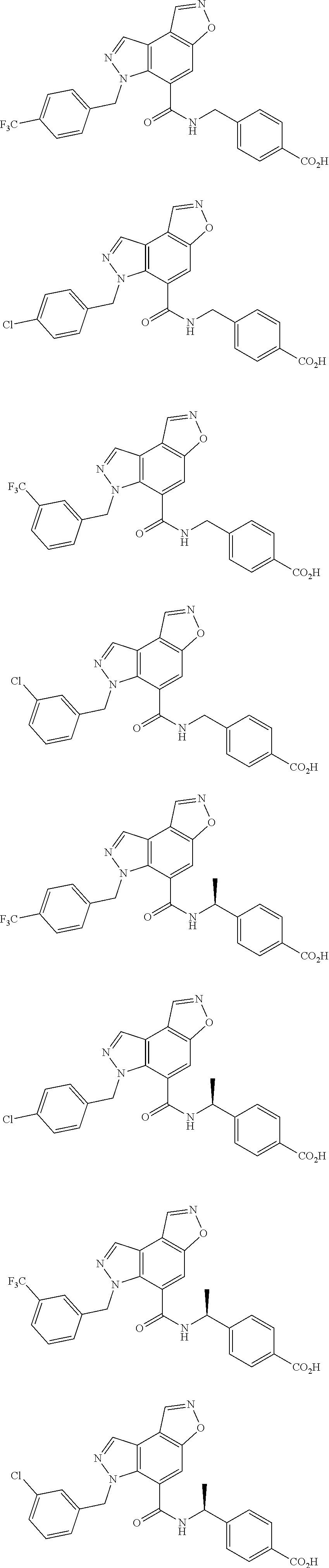

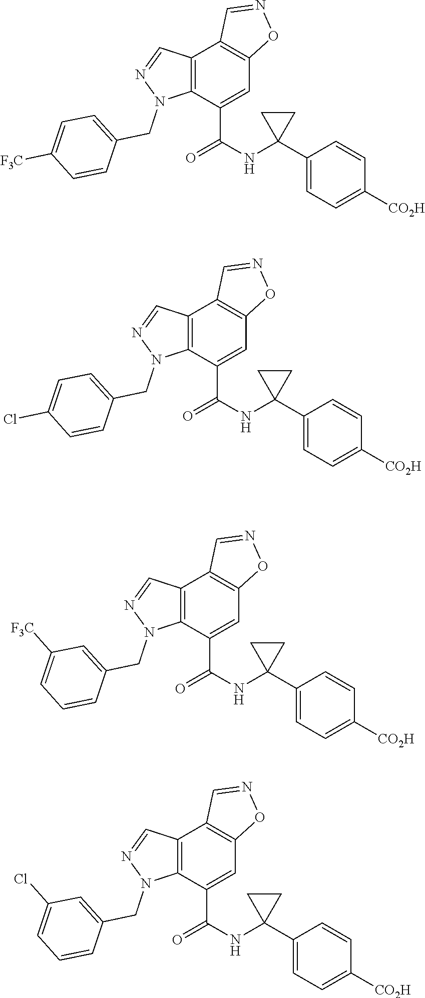 [SMILES: O=C(O)c1ccc(C2(NC(=O)c3cc4oncc4c4cnn(Cc5ccc(C(F)(F)F)cc5)c34)CC2)cc1 ; O=C(O)c1ccc(C2(NC(=O)c3cc4oncc4c4cnn(Cc5ccc(Cl)cc5)c34)CC2)cc1 ; O=C(O)c1ccc(C2(NC(=O)c3cc4oncc4c4cnn(Cc5cccc(C(F)(F)F)c5)c34)CC2)cc1 ; O=C(O)c1ccc(C2(NC(=O)c3cc4oncc4c4cnn(Cc5cccc(Cl)c5)c34)CC2)cc1]

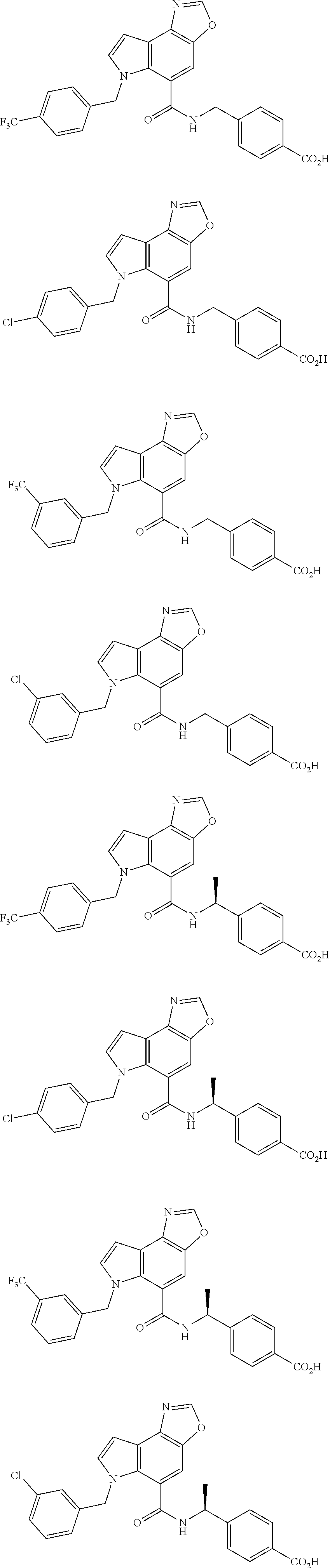

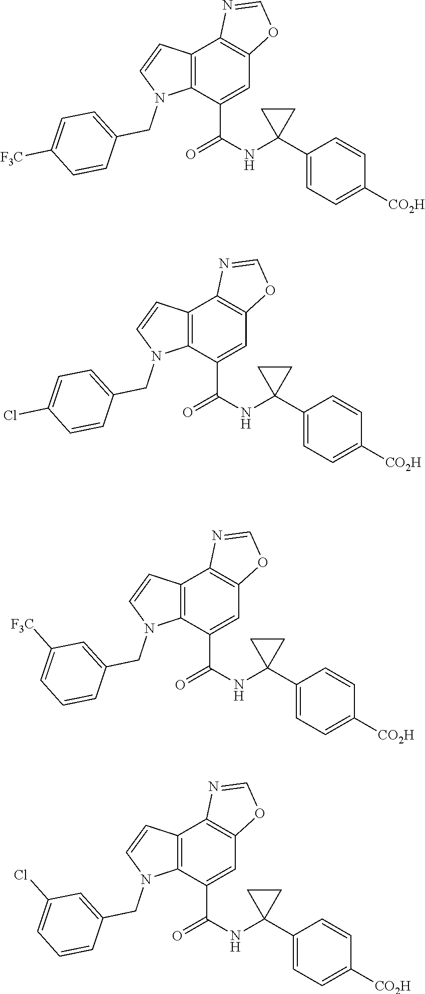 [SMILES: O=C(O)c1ccc(C2(NC(=O)c3cc4ocnc4c4ccn(Cc5ccc(C(F)(F)F)cc5)c34)CC2)cc1 ; O=C(O)c1ccc(C2(NC(=O)c3cc4ocnc4c4ccn(Cc5ccc(Cl)cc5)c34)CC2)cc1 ; O=C(O)c1ccc(C2(NC(=O)c3cc4ocnc4c4ccn(Cc5cccc(C(F)(F)F)c5)c34)CC2)cc1 ; O=C(O)c1ccc(C2(NC(=O)c3cc4ocnc4c4ccn(Cc5cccc(Cl)c5)c34)CC2)cc1]

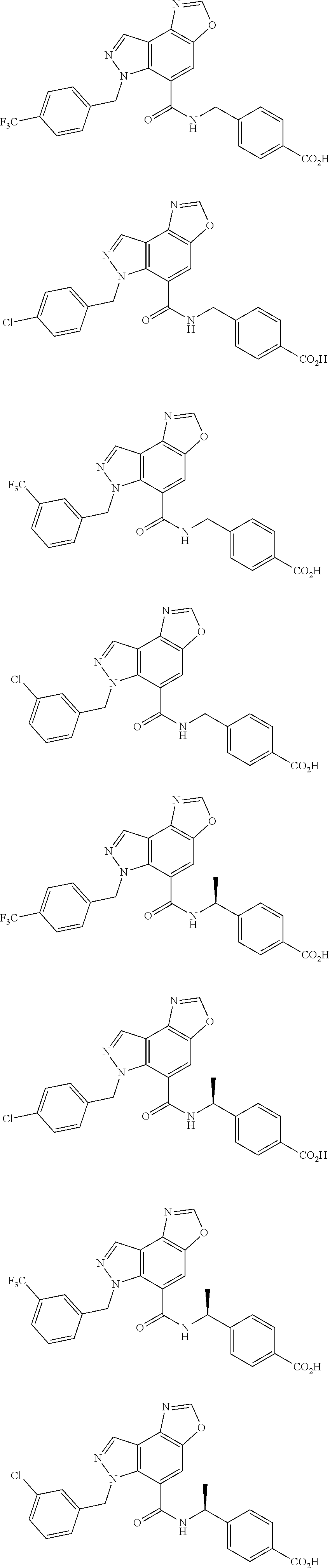

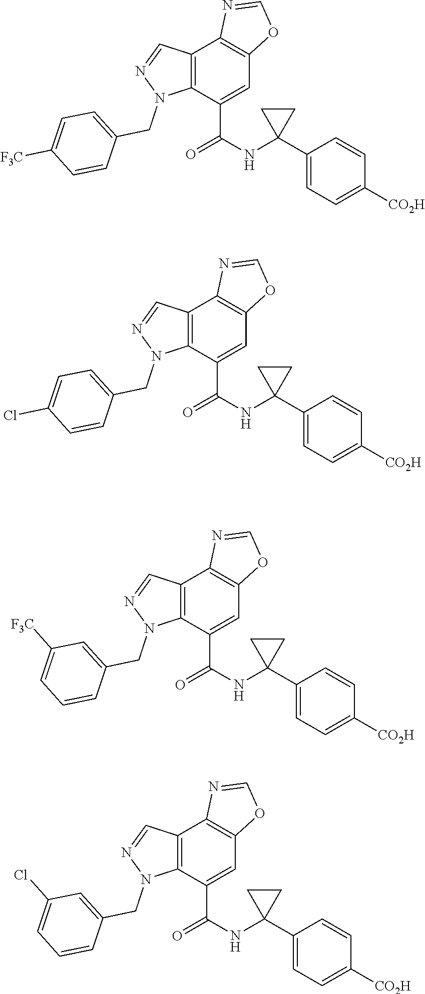 [SMILES: O=C(O)c1ccc(C2(NC(=O)c3cc4ocnc4c4cnn(Cc5ccc(C(F)(F)F)cc5)c34)CC2)cc1 ; O=C(O)c1ccc(C2(NC(=O)c3cc4ocnc4c4cnn(Cc5ccc(Cl)cc5)c34)CC2)cc1 ; O=C(O)c1ccc(C2(NC(=O)c3cc4ocnc4c4cnn(Cc5cccc(C(F)(F)F)c5)c34)CC2)cc1 ; O=C(O)c1ccc(C2(NC(=O)c3cc4ocnc4c4cnn(Cc5cccc(Cl)c5)c34)CC2)cc1]

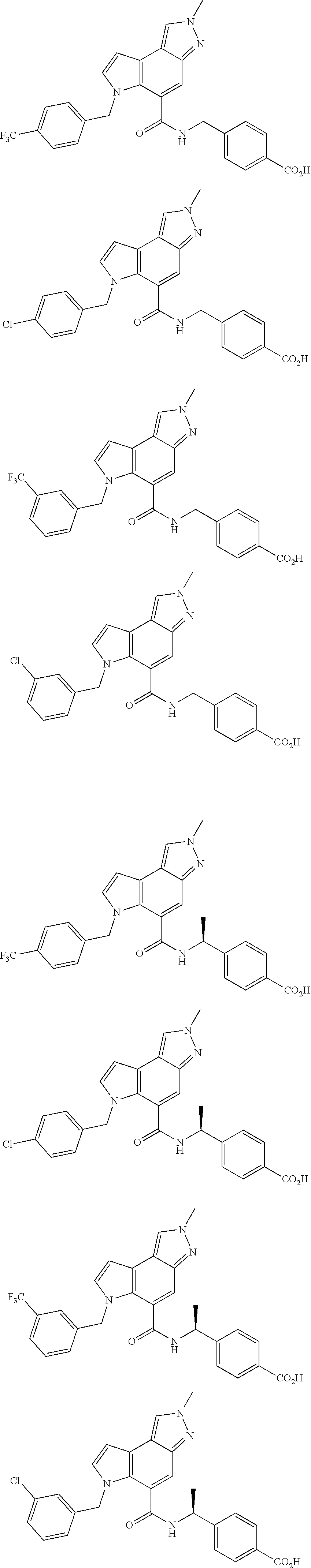

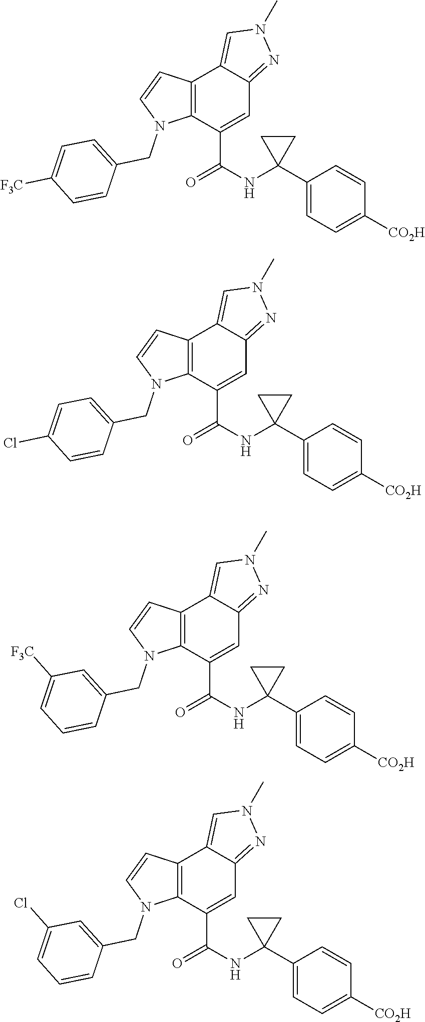 [SMILES: Cn1cc2c(cc(C(=O)NC3(c4ccc(C(=O)O)cc4)CC3)c3c2ccn3Cc2ccc(C(F)(F)F)cc2)n1 ; Cn1cc2c(cc(C(=O)NC3(c4ccc(C(=O)O)cc4)CC3)c3c2ccn3Cc2ccc(Cl)cc2)n1 ; Cn1cc2c(cc(C(=O)NC3(c4ccc(C(=O)O)cc4)CC3)c3c2ccn3Cc2cccc(C(F)(F)F)c2)n1 ; Cn1cc2c(cc(C(=O)NC3(c4ccc(C(=O)O)cc4)CC3)c3c2ccn3Cc2cccc(Cl)c2)n1]

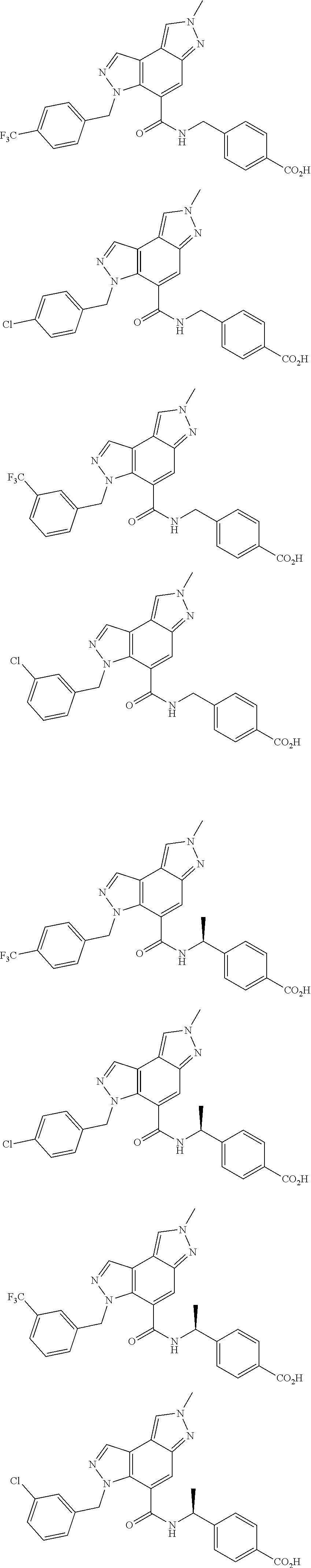

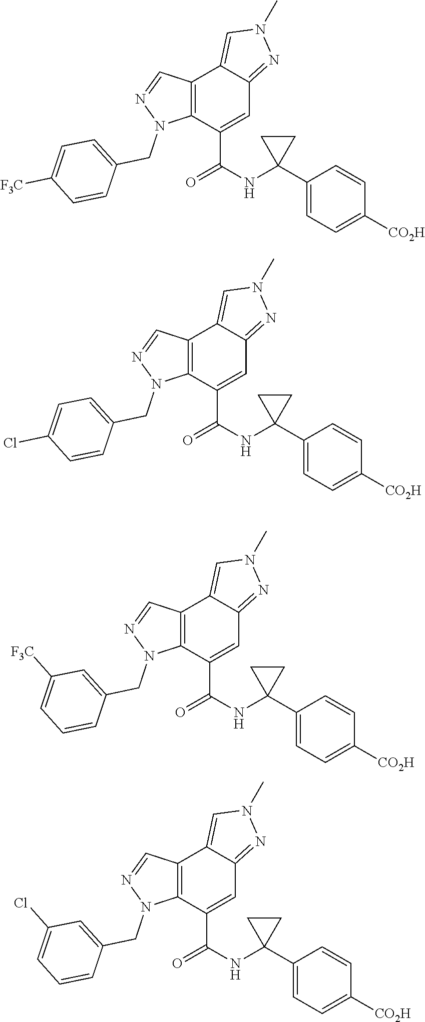 [SMILES: Cn1cc2c(cc(C(=O)NC3(c4ccc(C(=O)O)cc4)CC3)c3c2cnn3Cc2ccc(C(F)(F)F)cc2)n1 ; Cn1cc2c(cc(C(=O)NC3(c4ccc(C(=O)O)cc4)CC3)c3c2cnn3Cc2ccc(Cl)cc2)n1 ; Cn1cc2c(cc(C(=O)NC3(c4ccc(C(=O)O)cc4)CC3)c3c2cnn3Cc2cccc(C(F)(F)F)c2)n1 ; Cn1cc2c(cc(C(=O)NC3(c4ccc(C(=O)O)cc4)CC3)c3c2cnn3Cc2cccc(Cl)c2)n1]

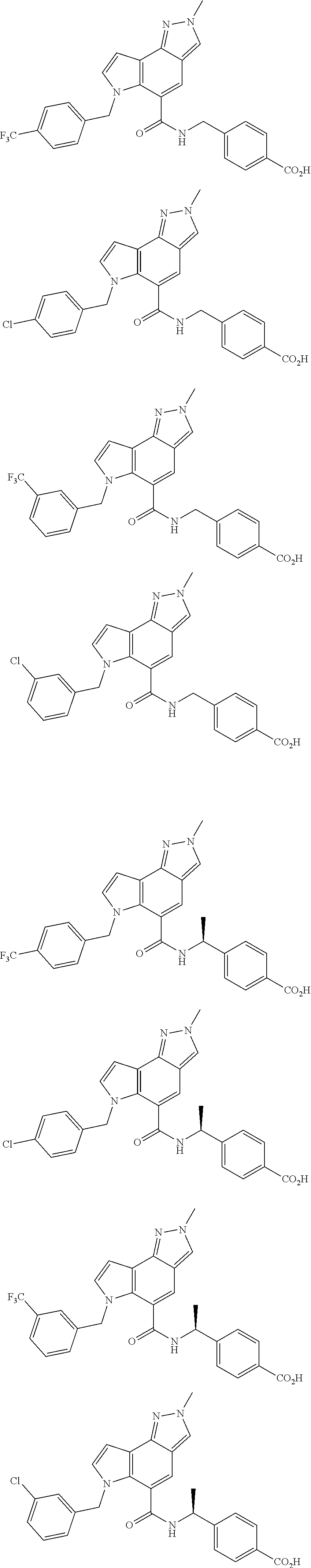

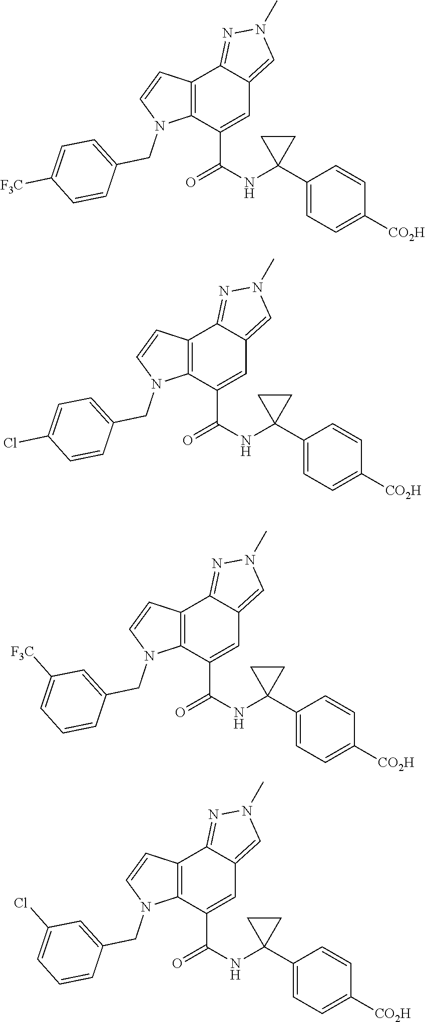 [SMILES: Cn1cc2cc(C(=O)NC3(c4ccc(C(=O)O)cc4)CC3)c3c(ccn3Cc3ccc(C(F)(F)F)cc3)c2n1 ; Cn1cc2cc(C(=O)NC3(c4ccc(C(=O)O)cc4)CC3)c3c(ccn3Cc3ccc(Cl)cc3)c2n1 ; Cn1cc2cc(C(=O)NC3(c4ccc(C(=O)O)cc4)CC3)c3c(ccn3Cc3cccc(C(F)(F)F)c3)c2n1 ; Cn1cc2cc(C(=O)NC3(c4ccc(C(=O)O)cc4)CC3)c3c(ccn3Cc3cccc(Cl)c3)c2n1]

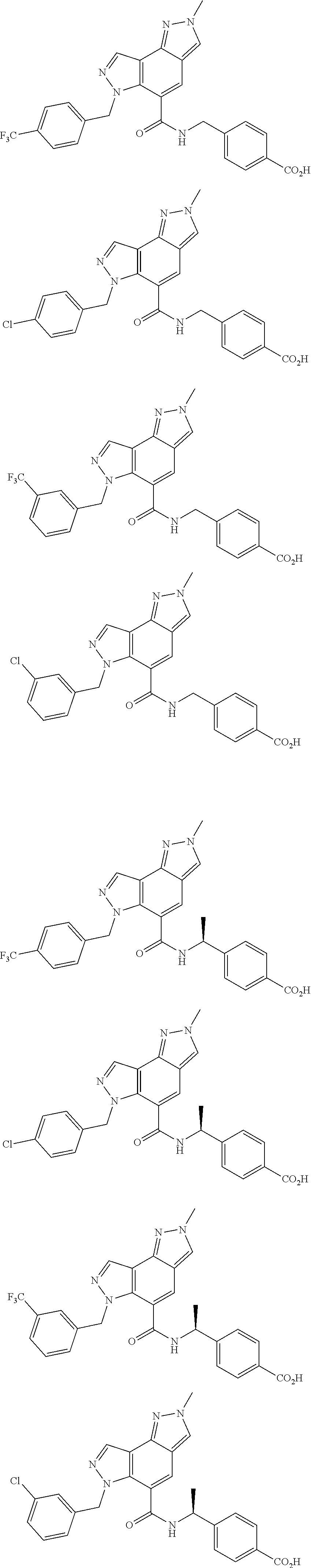

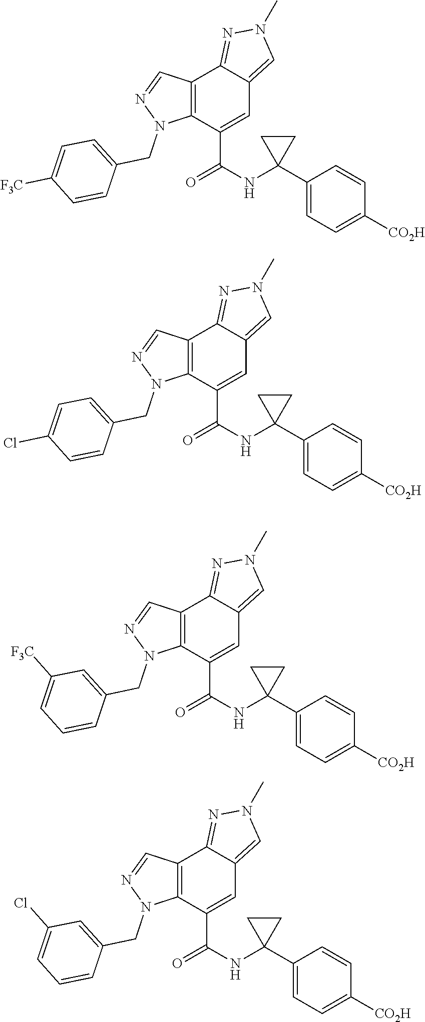 [SMILES: Cn1cc2cc(C(=O)NC3(c4ccc(C(=O)O)cc4)CC3)c3c(cnn3Cc3ccc(C(F)(F)F)cc3)c2n1 ; Cn1cc2cc(C(=O)NC3(c4ccc(C(=O)O)cc4)CC3)c3c(cnn3Cc3ccc(Cl)cc3)c2n1 ; Cn1cc2cc(C(=O)NC3(c4ccc(C(=O)O)cc4)CC3)c3c(cnn3Cc3cccc(C(F)(F)F)c3)c2n1 ; Cn1cc2cc(C(=O)NC3(c4ccc(C(=O)O)cc4)CC3)c3c(cnn3Cc3cccc(Cl)c3)c2n1]

The invention also encompasses a prodrug of a compound of the invention. The prodrug can be a choice of an ester or amide.

The invention also encompasses a pharmaceutical composition comprising a compound of the present invention in admixture with one or more physiologically acceptable carriers or excipients.

The invention also encompasses a compound of the invention or a pharmaceutically acceptable derivative thereof for use in human or veterinary medicine.

The invention also encompasses a method of treating a human or animal subject suffering from a condition which is mediated by the action of PGE2 receptors, which method comprises administering to said subject an effective amount of a compound of the invention.

The invention also encompasses the use of a compound of the invention for the manufacture of a therapeutic agent for the treatment of a condition which is mediated by the action of PGE2 receptors.

### Definitions

The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₁₋₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”. The alkyl is optionally substituted with one or more halogen atom(s).

The term “halogenated alkyl” means alkyl as defined above wherein one or more hydrogen atoms have been replaced by halogen atoms.

The term “alkenyl” means carbon chains which contain at least one carbon-carbon double bond, and which may be linear or branched, Z- or E-, or combinations thereof Examples of alkenyl are CH₃CH═CH—, CH₂═CHCH₂— and CH(CH₃)₂CH═CH—.

The term “alkylene” by itself or as part of another substituent means a divalent radical derived from an alkyl, as exemplified, but not limited, by —CH₂CH₂CH₂CH₂—, —CH═CH—, —CH₂CH═CHCH₂—, —CH₂C≡CCH₂—, —CH₂CH₂CH(CH₂CH₂CH₃)CH₂—. The alkylene radical may be optionally substituted independently with one or more substituents described herein, and includes radicals having “cis” and “trans” orientations, or alternatively, “E” and “Z” orientations. Typically, an alkyl (or alkylene) group has from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The alkylene is optionally substituted with one or more halogen atom(s).

The term “alkynyl” means carbon chains which contain at least one carbon-carbon triple bond, and which may be linear or branched or combinations thereof Examples of alkynyl include ethynyl, propargyl, 3-methyl-1-pentynyl, 2-heptynyl and the like. The alkynyl is optionally substituted with one or more halogen atom(s).

The term “alkylamino” refers to an amino substituent which is further substituted with one or two alkyl groups.

The term “aminoalkyl” refers to an alkyl substituent which is further substituted with one or more amino groups. The term “hydroxyalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

The term “cycloalkyl” or “carbococyclyl” means mono-, bicyclic or spiro-bicyclic carbocyclic rings, each of which has from 3 to 10 carbon atoms. A “fused analog” of cycloalkyl means a monocyclic ring fused to an aryl or heteroaryl group in which the point of attachment is on the non-aromatic portion.

Examples of cycloalkyl and fused analogs thereof include cyclopropyl, cyclobutyl, cyclopentyl, cyclohexyl, cycloheptyl, tetrahydronaphthyl, decahydronaphthyl, indanyl, and the like. The spiro-bicyclic carbocyclic rings are bicyclic (having just two rings) or have a bicyclic portion as part of the larger ring system, in either case with the two rings connected through the defining single common carbon atom. The cycloalkyl is optionally substituted with one or more halogen atom(s).

The term “alkoxy” means alkoxy groups of a straight or branched having the indicated number of carbon atoms. C₁₋₆alkoxy, for example, includes methoxy, ethoxy, propoxy, isopropoxy, and the like.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S —CH₂—CH₂— and —CH₂—S—CH₂—CH₂—NH—CH₂—.

For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR‘— and —R’OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “cycloalkoxy” means cycloalkyl as defined above bonded to an oxygen atom, such as cyclopropyloxy.

The term “halogenated alkoxy” means alkoxy as defined above wherein one or more hydrogen atoms have been replaced by halogen atoms.

The term “aryl” means mono- or bicyclic aromatic rings containing only carbon atoms. A “fused analog” of aryl means an aryl group fused to a monocyclic cycloalkyl or monocyclic heterocyclyl group in which the point of attachment is on the aromatic portion. Examples of aryl and fused analogs thereof include phenyl, naphthyl, indanyl, indenyl, tetrahydronaphthyl, 2,3-dihydrobenzofuranyl, dihydrobenzopyranyl, 1,4-benzodioxanyl, and the like.

The term “heteroaryl” means a mono- or bicyclic aromatic ring containing at least one heteroatom selected from N, O and S, with each ring containing 5 to 6 atoms. A “fused analog” of heteroaryl means a heteroaryl group fused to a monocyclic cycloalkyl or monocyclic heterocyclyl group in which the point of attachment is on the aromatic portion. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

The term “heterocyclyl” means mono-, bicyclic, tricyclic, spirocyclic, fused or bridged saturated ring systems containing at least one heteroatom selected from N, S and O, each of said ring having from 3 to 10 atoms in which the point of attachment may be carbon or nitrogen. A “heterocyclyl” includes a “fused analog” which means a monocyclic heterocycle fused to a heterocycle, a carbocycle, an aryl or heteroaryl group in which the point of attachment is on the non-aromatic portion. Examples of “heterocyclyl” and fused analogs thereof include azeridinyl, azetidinyl, pyrrolidinyl, piperidinyl, piperazinyl, imidazolidinyl, imidazopyridinyl, thiazolidinyl, dioxolanyl, oxazolodinyl, decahydroquinolinyl, piperidonyl, 4-piperidinonyl, quinuclidinyl, thiomorpholinyl, thiomorphonulyl 1,1-dioxide, morpholinyl, azapanyl, oxazepanyl, azabicyclohexanyls, azabicycloheptanyl, azabicyclooctanyl, azabycyclononanyl, azaspiroheptanyl, dihydro-1H,3H,5H-oxazolo[3,4-c]oxazolyl, tetrohydro-1′H3′Hspiro[cyclopropane-1,2′-pyrrolizinyl, hexahydro-1H-pyrralizinyl, hexahydro-1H-pyrrolo[2,1-c][1,4]oxazinyl, octahydroindolizinyl, oxaazaspirononanyls, oxaazspirooctanyls, diazaspirononanyls, oxaazabicycloheptanyls, hexahydropyrrolidinyl 4(lHO-oxide, tetrohydro-2H-thiopyranyl 1-oxide, tetrohydro-2H-thiopyranyl 1,1-dioxide, bicycloheptanyl, 2,3-dihydrofuro(2,3-b)pyridyl, benzoxazinyl, tetrahydrohydroquinolinyl, tetrahydroisoquinolinyl, hexahydro-]H-pyrrolizine, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

The said alkyl groups, cycloalkyl groups, heterocyclyl groups, aryl groups and heteroaryl groups referred to in the definitions are unsubstituted or are substituted by at least one substituent selected from the group consisting of substituents. The substituents are selected from the group consisting of halogen atoms, alkyl groups having from 1 to 4 carbon atoms, alkoxy groups having from 1 to 4 carbon atoms, haloalkyl groups having from 1 to 4 carbon atoms, haloalkoxy groups having from 1 to 4 carbon atoms, cyano groups, alkynyl groups having from 2 to 6 carbon atoms, alkanoyl groups having from 1 to 5 carbon atoms, cycloalkyl groups having from 3 to 7 ring atoms, heteroaryl groups, aryl groups, aralkoxy groups having from 7 to 10 carbon atoms, arylcarbonyl groups, two adjacent-x groups are optionally joined together to form an alkylene or an alkenylene chain having 3 or 4 carbon atoms, aminocarbonyl groups, alkenyl groups having from 2 to 5 carbon atoms, alkylthio groups having from 1 to 4 carbon atoms, aminosulfinyl groups, aminosulfonyl groups, hydroxy groups, —SF₅, —P(O)Me₂, hydroxyalkyl groups having from 1 to 4 carbon atoms, nitro groups, amino groups, carboxy groups, alkoxycarbonyl groups having from 2 to 5 carbon atoms, alkoxyalkyl groups having from 1 to 4 carbon atoms, alkylsulfonyl groups having from 1 to 4 carbon atoms, alkanoylamino groups having from 1 to 4 carbon atoms, alkanoyl(alkyl)amino groups having from 1 to 6 carbon atoms, alkanoylaminoalkyl groups having from 1 to 6 carbon atoms in both the alkanoyl and alkyl part, alkanoyl(alkyl)aminoalkyl groups having from 1 to 6 carbon atoms in both the alkanoyl and each alkyl part, alkylsulfonylamino groups having from 1 to 4 carbon atoms, mono- or di-alkylaminocarbonyl groups having from 1 to 6 carbon atoms, mono- or di-alkylaminosulfinyl groups having from 1 to 6 carbon atoms, mono- or di alkylaminosulfonyl groups having from 1 to 6 carbon atoms, aminoalkyl groups having from 1 to 4 carbon atoms, mono- or di-alkylamino groups having from 1 to 6 carbon atoms, mono- or di-alkylaminoalkyl groups having from 1 to 6 carbon atoms in each alkyl part, aralkyl groups having from 7 to 10 carbon atoms, heteroarylalkyl groups having from 1 to 4 carbon atoms in the alkyl part, heteroarylalkoxy groups having from 1 to 4 carbon atoms in the alkoxy part and alkylsulfonylamino groups having from 1 to 4 carbon atoms.

The terms “halo” or “halogen,” by themselves or as part of another substituent, mean, unless otherwise stated, a fluorine, chlorine, bromine, or iodine atom. Additionally, terms such as “haloalkyl,” or “halogenated alkyl” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrugs may also have improved solubility in pharmaceutical compositions over the parent drug. An example, without limitation, of a prodrug would be a compound of any of Formula I, which is administered as an ester (the “prodrug”) to facilitate transmittal across a cell membrane where water solubility is detrimental to mobility, but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

### Optical Isomers—Diastereomers—Geometric Isomers—Tautomers

Compounds of the present invention may contain one or more asymmetric centers and may thus occur as racemates and racemic mixtures, single enantiomers, diastereomeric mixtures and individual diastereomers. The present invention is meant to comprehend all such isomeric forms of the compounds.

Some of the compounds described herein contain olefinic double bonds, and unless specified otherwise, are meant to include both E and Z geometric isomers.

Some of the compounds of described herein may contain one or more than one cyclic ring systems and may thus exist in cis- and trans-isomers. The present invention is meant to include all such cis- and trans-isomers.

Some of the compounds described herein may exist with different points of attachment of hydrogen, referred to as tautomers. Such an example may be a ketone and its enol form known as keto-enol tautomers.

The individual tautomers as well as mixture thereof are encompassed with compounds of the present invention.

Compounds described herein may be separated into diastereoisomeric pairs of enantiomers by, for example, fractional crystallization from a suitable solvent, for example MeOH or EtOAc or a mixture thereof The pair of enantiomers thus obtained may be separated into individual stereoisomers by conventional means, for example by the use of an optically active amine or acid as a resolving agent or on a chiral HPLC column.

Alternatively, any enantiomer of a compound described herein may be obtained by stereospecific synthesis using optically pure starting materials or reagents of known configuration.

### Stable Isotope—Labeled Analogs

One or more than one of the protons (hydrogen atoms) in compounds of the present invention can be replaced with deuterium atom(s), thus providing deuterated analogs that may have improved pharmacokinetic properties or pharmacological activities.

### Salts and Formulations

The compounds described herein can be useful as the free base or as a salt.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydramine, isopropylamine, lysine, methylglucamine, morpholine, piperazine, piperidine, polyamine resins, procaine, purines, theobromine, triethylamine, trimethylamine, tripropylamine, tris(hydroxymethyl)aminomethane, tromethamine, and the like.

When the compound of the present invention is alkaline, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of the present invention are meant to also include the pharmaceutically acceptable salts.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients are mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methylcellulose, hydroxypropylmethy-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomiser (preferably an I atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules (made, for example, from gelatin or HPMC), blisters and cartridges for use in an inhaler or insufflator may be formulated to contain a powder mix of the compound of the invention, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 mg to 20 mg of the compound of the invention per actuation and the actuation volume may vary from 1 L to 100 L. A typical formulation may comprise a compound of Formula (A) propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 pg to 1 mg of the compound of the present invention. The overall daily dose will typically be in the range 1 pg to 1 mg which may be administered in a single dose or, more usually, as divided doses throughout the day.

Compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of the present invention are employed. (For purposes of this application, topical application shall include mouth washes and gargles.)

Dosage levels of the order of from about 0.01 mg to about 140 mg/kg of body weight per day are useful in the treatment of the above-indicated conditions, or alternatively about 0.5 mg to about 7 g per patient per day. For example, a condition may be effectively treated by the administration of from about 0.01 to 50 mg of the compound per kilogram of body weight per day, or alternatively about 0.5 mg to about 3.5 g per patient per day, preferably 2.5 mg to 1 g per patient per day.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

### Utilities

The compounds of the invention are antagonists of PGE2 receptors and are therefore useful in treating a PGE2 mediated disease or condition.

The invention also encompasses a method of treating cancer with an effective amount of a compound of the present invention or using a combination of an effective amount of a compound of the present invention with an effective amount of radiation, chemotherapies, PI3K/AKT pathway inhibitors, endocrine/hormone therapeutics, immunotherapies such as antibodies to cytotoxic t-lymphocyte antigen 4 (anti-CTLA4), programmed death ligand 1 (anti-PDLl), to programmed cell death protein 1 (anti-PD₁), indoleamine-2,3-dioxygenase (IDO) inhibitors, tryptophan-2,3-dioxygenase (TDO) inhibitors, EP1/EP3 agonists and antimetabolites.

In yet another aspect of the invention, the cancer treated is selected from the group consisting of breast cancers, cervical cancers, colorectal cancers, endometrial cancers, glioblastomas, head and neck cancers, kidney cancers, liver cancers, lung cancers, medulloblastomas, ovarian cancers, pancreatic cancers, prostate cancers, skin cancers and urinary tract cancers. In more particular aspects of the invention, provided are methods of treating cancer and/or generating a memory immune response comprising administering a compound of the present invention.

### Synthesis

The compounds of the present invention can be prepared according to the following synthetic schemes:

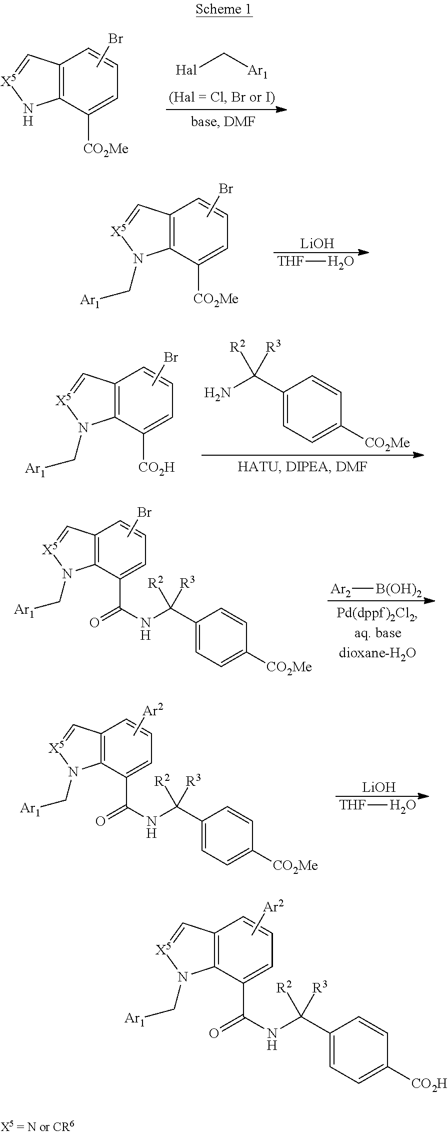

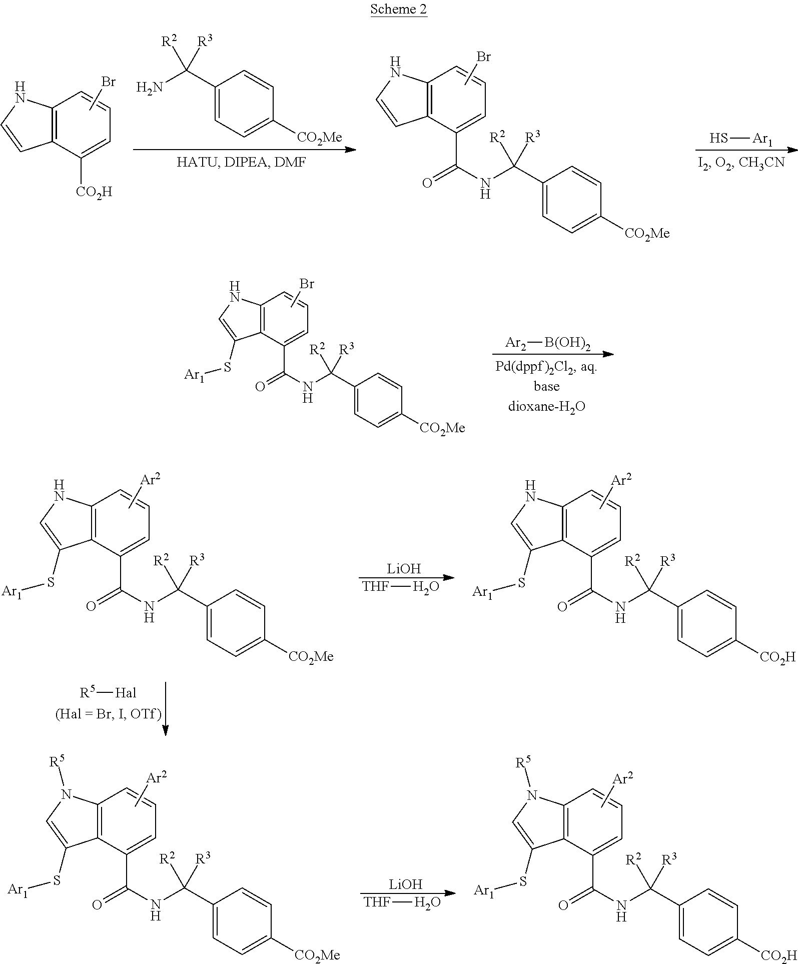

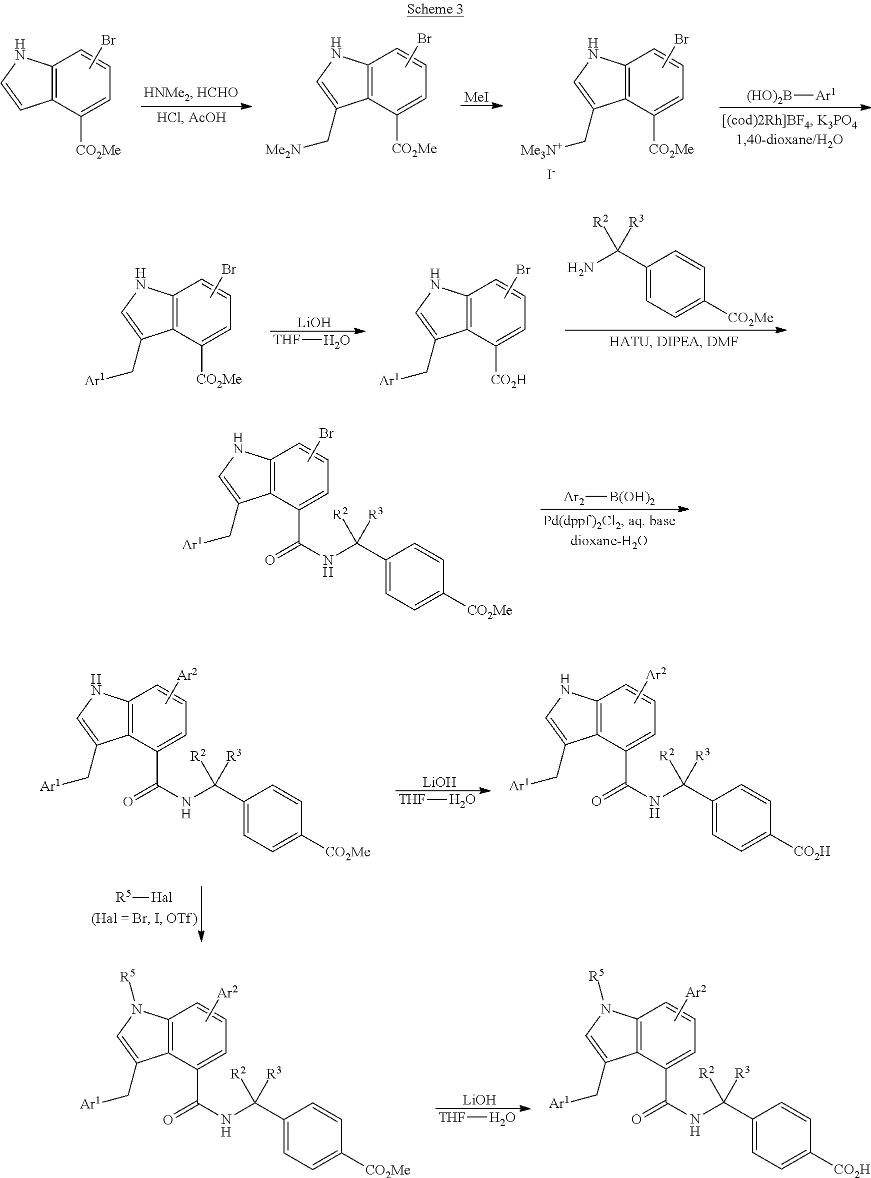

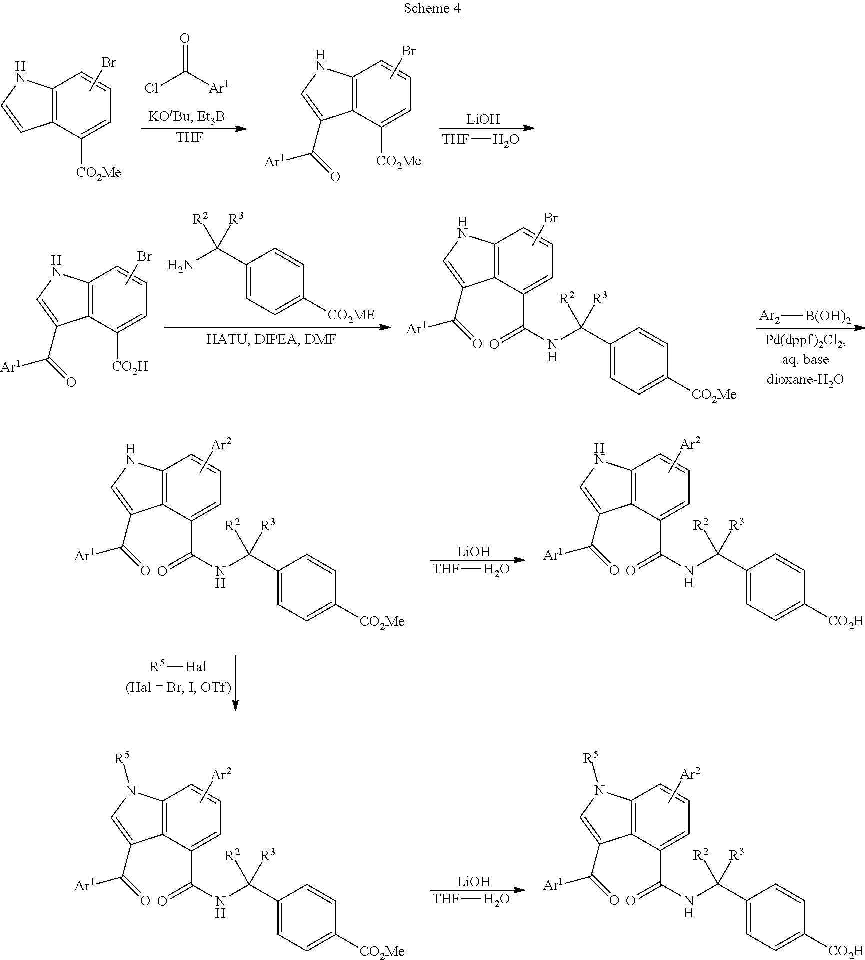

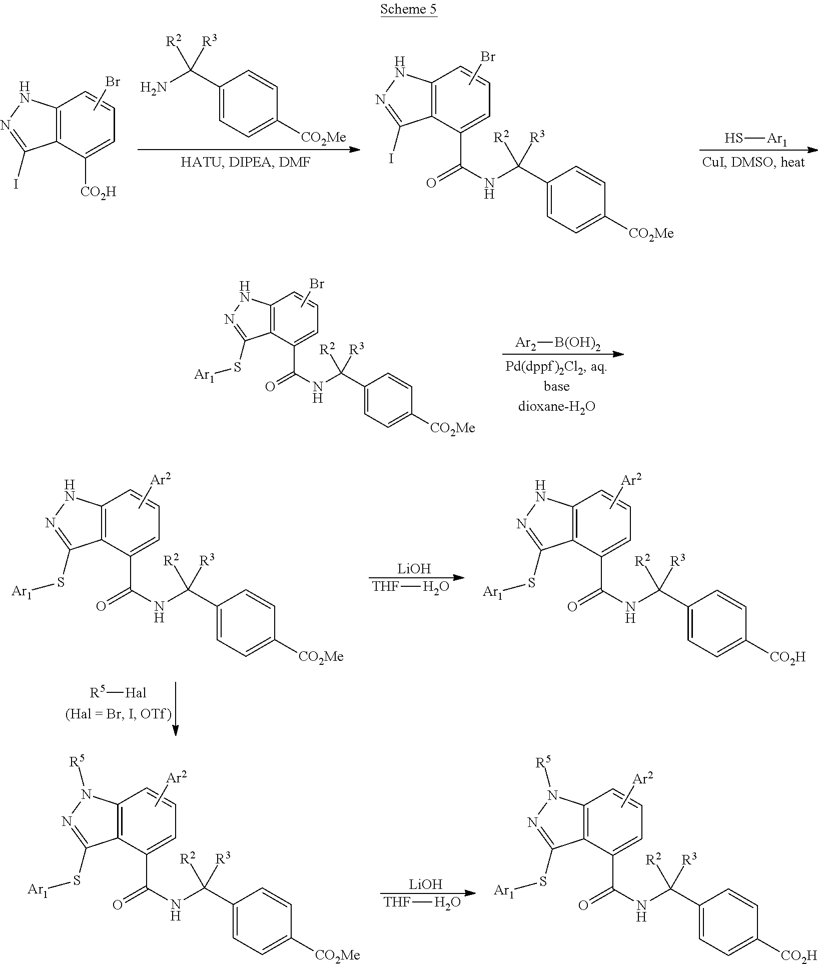

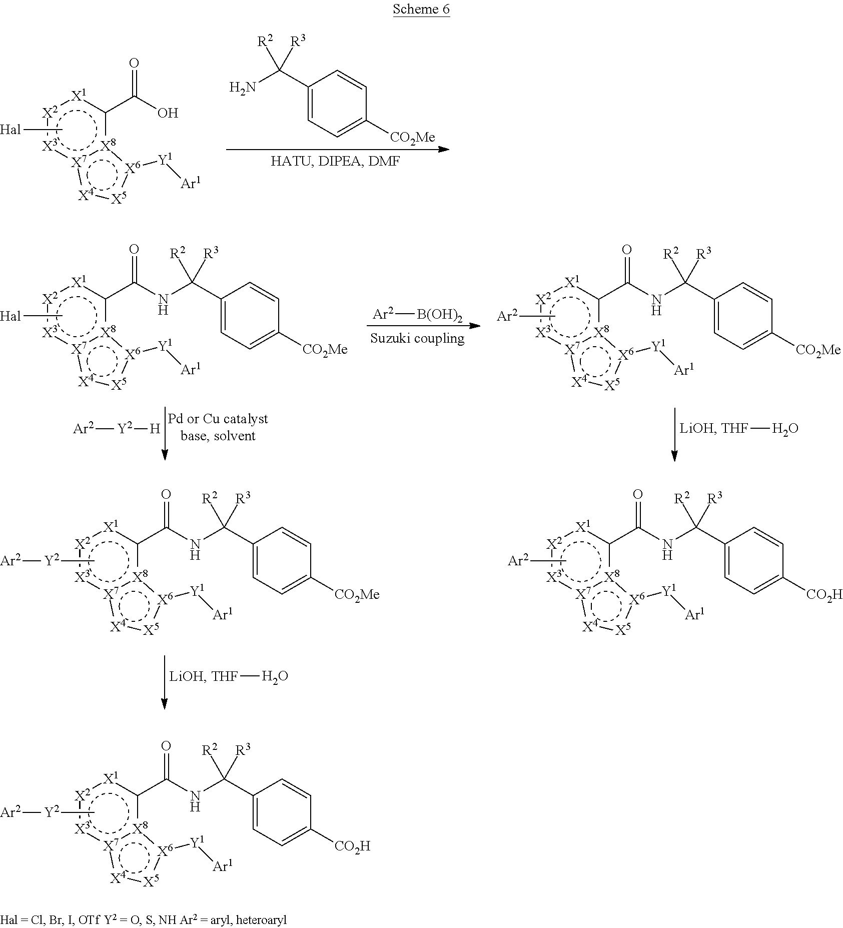

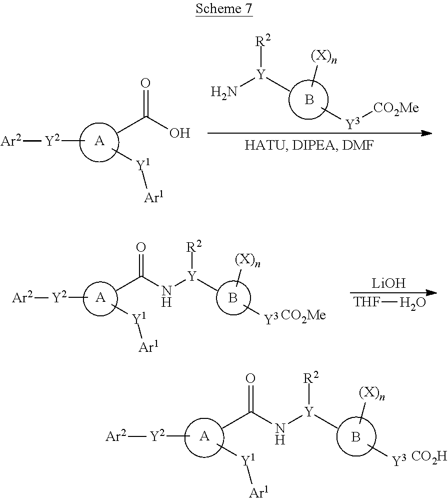

### Evaluation of Biological Activity

1. The HEK-293 CNG (Cyclic Nucleotide Gated) cell line expressing a modified CNG channel that leads to cell membrane depolarization in response to elevated intracellular cAMP levels, which can be measured using a fluorescent membrane potential dye, was engineered to express human EP2 (PTGER2) or EP4 (EP4R). The resulting cell line ACTOne™ PTGER2 or ACTOne™ EP4R as well as the fluorescent membrane potential dye were purchased from eEnzyme (Gaithersburg, MD, US) and used in this assay. Briefly, cells were plated at 1.2×10⁴ cells/well in 20 μL DMEM supplemented with 2% Fetal Bovine Serum (FBS, VWR International), 1x Penicillin/Streptomycin (GenClone), 0.2 μg/mL puromycin (InvivoGen) and 50 μg/mL G418 (InvivoGen) on a 384 well plate (Greiner). The next day, cells were dye-loaded with 20 μL/well of 1x Dye-loading solution (eENZYME) following the manufacturer's instruction. After 2 hours of incubation at room temperature (RT) in the dark, a pre-read was made to obtain the background signals using CLARIOstar plate reader (BMG LABTECH, Germany) with the following filter settings: excitation=530/20 nm, emission=590/20 nm. Next, a serial dilution of compound (plus a DMSO control) were added to the plate (5 μL/well) and the reaction was incubated at RT for 15 min before adding 5 μL of PGE2 to every well for a final concentration of 5 nM. All wells were duplicated. The plate was then further incubated at RT for 30 min prior to a final read with the same machine settings. Background-subtracted signals were used to calculate IC₅₀ values.

2. Human EP2 and EP4 Reporter Assay Kits were purchased from Cayman Chemical (Ann Arbor, MI, USA). The assay was performed following the provided User Manual. Briefly, a reverse transfection was performed by seeding 6.0×10⁴ of 293T cells (ATCC) to each well of the provided Strip Plate coated with a proprietary transfection complex containing DNA constructs for expressing human EP2 or EP4 and a cAMP response element regulated secreted alkaline phosphatase (SEAP) reporter, in 200 μL DMEM supplemented with 2% FBS and incubated for 20-24 hours at 37° C. with 5% CO₂. After the incubation, the culture medium was carefully removed, and the cells were replenished with 150 μL pre-warmed serum-free medium. PGE2 and test compounds were prepared together at 4x the desired final concentration, and 50 μL was added to each assigned well with the final concentration of 13 nM and 2 nM PGE2 for EP2 and EP4 assay, respectively. After 6-8 hours of incubation, 10 μL media from each well was transferred to corresponding well of a new plate, which was then sealed and incubated at 65° C. for 30 min. The plate was then cooled down to RT and 50 μL SEAP substrate was added to each well. Luminescence signal was measured using a CLARIOstar plate reader.

Table 1 shows the activity of selected compounds of this invention in the EP2 antagonist assays. The IC₅₀ value was determined as the concentration for 50% inhibition of signal compared to DMSO (A: IC_{50 5} 100 nM; B: IC₅₀ between 100 nM and 1,000 nM; C: IC₅₀≥1,000 nM).

TABLE 1
EP2 antagonist activity

| Example | EP2 IC₅₀ |
|---|---|
| 1 | A |
| 2 | A |
| 3 | C |
| 4 | C |
| 5 | B |
| 6 | B |
| 7 | A |
| 8 | A |
| 9 | A |
| 10 | B |
| 11 | A |
| 12 | C |
| 13 | C |
| 14 | C |
| 15 | C |
| 16 | C |
| 17 | A |
| 18 | B |
| 19 | C |
| 20 | C |
| 21 | B |
| 22 | B |
| 23 | C |
| 24 | C |
| 25 | C |
| 26 | B |
| 27 | C |
| 28 | C |
| 29 | C |
| 30 | C |
| 31 | C |
| 32 | C |
| 33 | C |
| 34 | C |
| 35 | C |
| 36 | C |
| 37 | C |
| 38 | C |
| 39 | B |
| 40 | C |
| 41 | C |
| 42 | C |
| 43 | A |
| 44 | A |
| 45 | B |
| 46 | C |
| 47 | C |
| 48 | C |
| 49 | B |
| 50 | A |
| 51 | A |
| 52 | A |
| 53 | A |
| 54 | A |
| 57 | A |
| 58 | C |
| 59 | C |
| 61 | B |
| 62 | C |
| 63 | B |
| 64 | A |
| 65 | B |
| 66 | B |
| 67 | A |
| 68 | A |
| 69 | A |
| 70 | A |
| 71 | B |
| 72 | A |
| 73 | B |
| 74 | A |
| 75 | A |
| 76 | A |
| 77 | A |
| 78 | A |
| 79 | B |
| 80 | A |
| 81 | A |
| 82 | A |
| 83 | A |
| 84 | A |
| 85 | B |
| 86 | A |
| 87 | A |
| 88 | B |
| 89 | B |
| 90 | A |
| 91 | A |
| 92 | A |
| 93 | B |
| 94 | A |
| 95 | A |
| 96 | A |
| 97 | A |
| 98 | A |
| 99 | A |
| 100 | A |
| 101 | A |
| 102 | A |
| 103 | B |
| 104 | A |
| 105 | A |
| 106 | A |
| 107 | A |
| 108 | A |
| 109 | A |
| 110 | A |
| 111 | A |
| 112 | B |
| 113 | A |
| 114 | A |
| 115 | B |
| 116 | B |
| 117 | A |
| 118 | B |
| 119 | B |
| 120 | B |
| 121 | B |
| 122 | A |
| 123 | B |
| 124 | A |
| 125 | A |
| 126 | A |
| 127 | A |
| 128 | A |
| 129 | A |
| 130 | A |
| 131 | B |
| 132 | B |
| 133 | B |
| 134 | A |
| 135 | A |
| 136 | A |
| 137 | A |
| 138 | A |
| 139 | A |
| 140 | B |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | A |
| 174 | A |
| 175 | A |
| 176 | A |
| 177 | A |
| 178 | A |
| 179 | A |
| 180 | A |
| 181 | C |
| 182 | C |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | A |
| 197 | B |
| 198 | A |
| 199 | A |
| 200 | A |
| 201 | A |
| 202 | A |
| 203 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | C |
| 224 | A |

Table 2 shows the activity of selected compounds of this invention in the EP4 antagonist assay. The IC₅₀ value was determined as the concentration for 5000 inhibition of signal compared to DMSO (A: IC₅₀≤100 nM; B: IC₅₀ between 100 nM and 1,000 nM; C: IC₅₀≥1,000 nM).

TABLE 2
EP4 antagonist activity

| Example | EP4 IC₅₀ | Example | EP4 IC₅₀ | Example | EP4 IC₅₀ |
|---|---|---|---|---|---|
| 1 | A | 100 | A | 152 | A |
| 2 | A | 101 | A | 154 | A |
| 7 | A | 102 | A | 156 | A |
| 9 | A | 106 | A | 165 | A |
| 44 | A | 107 | A | 166 | A |
| 50 | A | 117 | A | 168 | A |
| 51 | A | 124 | A | 169 | A |
| 52 | A | 129 | A | 175 | A |
| 57 | B | 134 | A | 176 | A |
| 69 | B | 135 | A | 180 | A |
| 74 | A | 136 | A | 184 | A |
| 76 | A | 137 | A | 188 | A |
| 78 | A | 224 | A | 189 | A |
| 80 | A | 141 | A | 190 | A |
| 85 | A | 147 | A | 191 | A |
| 87 | A | 149 | A | 221 | A |
| 96 | A | 150 | A | 223 | A |
| 97 | A | 151 | A | 224 | A |

### Abbreviations

The following abbreviations have the meanings indicated. EA means ethyl acetate; CIP means 2-chloro-1,3-dimethylimidazolidium hexafluorophosphate; DBU means 1,8-diazabicyclo[5.4.0]undec-7-ene; DIBAL means diisobutylaluminum hydride; DCM means dichloromethane; DIEA or DIPEA means diisopropylethylamine; DMAP means N,N-dimethylaminopyridine; DME means 1,2-dimethoxyethane; DMF means N,N-dimethylformamide; dmpe means 1,2-bis(dimethylphosphino)ethane; DMSO means dimethylsulfoxide; dppb means 1,4-bis(diphenylphosphino)butane; dppe means 1,2-bis(diphenylphosphino)ethane; dppf means 1,1′-bis(diphenylphosphino)ferrocene; dppm means 1,1′-bis(diphenylphosphino)methane; DIAD means diisopropylazodicarboxylate; EDCI means 1-(3-dimethylaminopropyl)-3-ethylcarbodiimide; HATU means 2-(7-Aza-1H-benzotriazole-1-yl)-1,1,3,3-tetramethyluronium hexafluorophosphate; HMPA means hexamethylphosphorarnide; IPA means isopropyl alcohol; LDA means lithium diisopropylamide; LHMDS means lithium bis(hexamethyldisilylamide); LAH means lithium aluminum hydride; NCS means N-chlorosuccinimide; PE means petroleum ether; PyBOP means benzotriazol-1-yloxytripyrrolidinophosphonium hexafluorophosphate; SGC means silica gel chromatography; TDA means tris(2-(2-methoxyethoxy)ethyl)amine; DCM means dichloromethame; TEA means triethylamine; TFA means trifluoroacetic acid; THF means tetrahydrofuran; NCS means N-chlorosuccinimide; NMI means 1-methylimidazole; NMM means N-methylmorpholine; NMP means N-methylpyrrolidine; PPh3 means triphenylphosphine; rt or RT means room temperature; sat. means saturated; T3P means propylphosphonic anhydride; TCFH means N,N,N′,N′-tetramethylchloroformaidinium hexafluorophosphate; MTBE means methyl tert-butyl ether; NBS means N-bromosuccinimide; DMA means N,N-Dimethylacetamide,*in structures means undetermined R or S chiral center.

LCMS conditions:
Column: Xtimate C18, 3 m, 4.6×50 mm.
Mobile phase: 0.03% TFA in water and 0.03% TFA in CH₃CN;
Gradient: 5% CH₃CN to 95% CH₃CN in 6.5 min;
Flow rate: 1 mL/min.;
MS instrument: Waters UPLC-PDA-3100 (SQD3100) single-quadrupole mass spectrometer.

The present invention will be more readily understood by referring to the following examples which are given to illustrate the invention rather than to limit its scope.

### EXAMPLE 1

### 4-((5-(4-fluorophenyl)-1-(4-(trifluoromethyl)benzyl)-1H-indole-7-carboxamido)methyl)benzoic acid

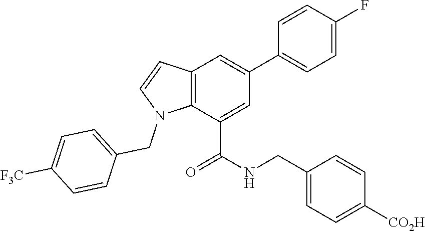 [SMILES: O=C(O)c1ccc(CNC(=O)c2cc(-c3ccc(F)cc3)cc3ccn(Cc4ccc(C(F)(F)F)cc4)c23)cc1]

### Step 1 methyl 5-bromo-1-(4-(trifluoromethyl)benzyl)-1H-indole-7-carboxylate

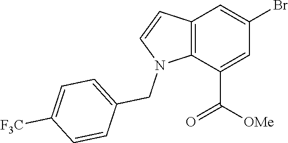 [SMILES: COC(=O)c1cc(Br)cc2ccn(Cc3ccc(C(F)(F)F)cc3)c12]

To a mixture of methyl 5-bromo-1H-indole-7-carboxylate (500 mg, 1.97 mmol) and Cs₂CO₃ (769 mg, 2.36 mmol) in anhydrous DMF (10 mL) at 0° C. was added 1-(bromomethyl)-4-(trifluoromethyl)benzene (564 mg, 2.36 mmol). The reaction mixture was then warmed to rt and stirred overnight. The reaction mixture was poured into 100 mL ice-water, extracted 3×50 mL ethyl acetate. The combined organic layer was washed with 50 mL saturated brine, dried over anhydrous Na₂SO₄ and concentrated. The residue was purified by silica gel column matography (petroleum ether/ethyl acetate: from 100:0 to 10:1) to afford the title compound as a white solid (500 mg). LCMS (ES+): 412.20 [M+1]⁺.

### Step 2 5-bromo-1-(4-(trifluoromethyl)benzyl)-1H-indole-7-carboxylic acid

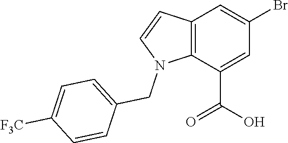 [SMILES: O=C(O)c1cc(Br)cc2ccn(Cc3ccc(C(F)(F)F)cc3)c12]

To a solution of the product of Step 1 (500 mg, 1.21 mmol) in MeOH/H₂O (10 mL/2.0 mL) was added LiOH—H₂O (254 mg, 6.06 mmol) at rt. The reaction mixture was stirred at 60° C. for 1 h. After being cooled to 0° C., the reaction mixture was treated with 6 N HCl to adjusted pH≈6. The reaction mixture was extracted 3×30 mL EtOAc. The combined organic layer washed with 20 mL saturated brine, dried over anhydrous Na₂SO₄, concentrated. The residue was purified by silica gel column chromatography (DCM/MeOH: from 100:0 to 10:1) to afford the title compound as a white solid (479 mg). LCMS (ESI): 398.22 [M+1]⁺.

### Step 3 methyl 4-((5-bromo-1-(4-(trifluoromethyl)benzyl)-1H-indole-7-carboxamido)methyl)benzoate

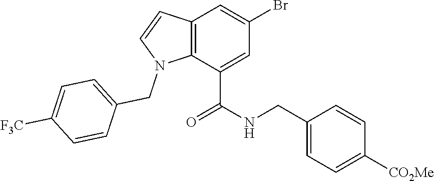 [SMILES: COC(=O)c1ccc(CNC(=O)c2cc(Br)cc3ccn(Cc4ccc(C(F)(F)F)cc4)c23)cc1]

To a mixture of the product of Step 2 (398 mg, 1.0 mmol), methyl 4-(aminomethyl)benzoate (215 mg, 1.3 mmol) and HATU (456 mg, 1.2 mmol) in anhydrous DMF (100 mL) at 0° C. was added DIPEA (0.52 mL, 3.0 mmol). After stirring at rt overnight, the reaction mixture was quenched with 100 mL water and extracted 3×100 mL EtOAc. The combined organic layer was washed with 100 mL saturated brine, dried over anhydrous Na₂SO₄ and concentrated. The residue was purified by silica gel column chromatography (petroleum ether/ethyl acetate: from 100:0 to 5:1) to afford the title compound as a white solid (0.5 g). LCMS (ESI): 547.29 [M+1]⁺.

### Step 4 methyl 4-((5-(4-fluorophenyl)-1-(4-(trifluoromethyl)benzyl)-1H-indole-7-carboxamido)methyl)benzoate

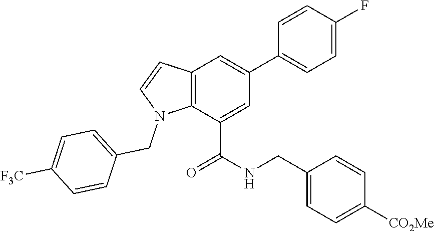 [SMILES: COC(=O)c1ccc(CNC(=O)c2cc(-c3ccc(F)cc3)cc3ccn(Cc4ccc(C(F)(F)F)cc4)c23)cc1]

A mixture of the product of Step 3 (30 mg, 0.06 mmol), (4-fluorophenyl)boronic acid (38 mg, 0.28 mmol), Pd(dppf)₂Cl₂ (4 mg, 0.01 mmol), K₂CO₃ (38 mg, 0.17 mmol) in EtOH/H₂O (5 ml/0.05 mL) was stirred for 2 h at 70° C. under nitrogen. The reaction mixture was concentrated and purified by silica gel column chromatography (PE/EA=5:1) to afford the title compound as a yellow solid (27 mg). LCMS (ESI): 561.49 [M+1]⁺.

### Step 5 4-((5-(4-fluorophenyl)-1-(4-(trifluoromethyl)benzyl)-1H-indole-7-carboxamido)methyl)benzoic acid

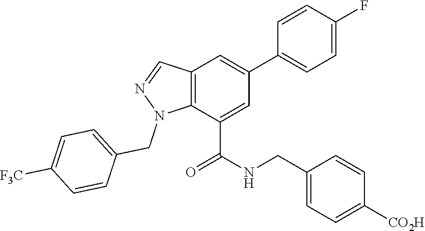 [SMILES: O=C(O)c1ccc(CNC(=O)c2cc(-c3ccc(F)cc3)cc3cnn(Cc4ccc(C(F)(F)F)cc4)c23)cc1]

To a solution of the product of Step 4 (42 mg, 0.07 mmol) in MeOH/H₂O (5 mL/0.5 mL) was added LiOH—H₂O (16 mg, 0.37 mmol). The reaction mixture was stirred at 60° C. for 1 h. After being cooled to 0° C., the mixture was treated with 6 N HCl to adjusted pH≈6. The reaction mixture was then extracted 3×20 mL EtOAc. The combined organic layer was washed with 20 mL saturated brine, dried over anhydrous Na₂SO₄ and concentrated. The residue was purified by silica gel column chromatography (DCM/MeOH=10:1) to afford the title compound as a white solid (10.1 mg). LCMS (ESI): 547.40 [M+1]⁺.¹HNMR Spectrum: (500 MHz, DMSO-d₆) δ: 9.00 (t, J=5.9 Hz, 1H), 7.99 (d, J=1.7 Hz, 1H), 7.83 (d, J=8.1 Hz, 2H), 7.79-7.70 (m, 2H), 7.63 (d, J=3.1 Hz, 1H), 7.56-7.48 (m, 3H), 7.29 (dt, J=8.9, 4.8 Hz, 4H), 6.98 (d, J=8.0 Hz, 2H), 6.72 (d, J=3.2 Hz, 1H), 5.72 (s, 2H), 4.41 (d, J=5.9 Hz, 2H).

### EXAMPLE 2

### 4-((5-phenyl-1-(4-(trifluoromethyl)benzyl)-1H-indole-7-carboxamido)methyl)benzoic acid

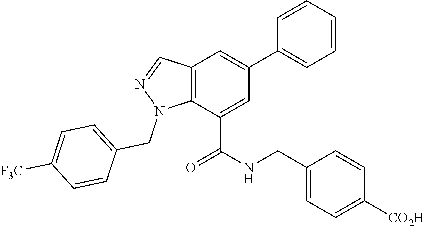 [SMILES: O=C(O)c1ccc(CNC(=O)c2cc(-c3ccccc3)cc3cnn(Cc4ccc(C(F)(F)F)cc4)c23)cc1]

Starting with phenylboronic acid (15 mg) and the product of Step 3 of Example 1 (30 mg), the title product was obtained as a white solid (9 mg) by following the same procedures as described in Step 4 and Step 5 of Example 1. LCMS (ESI): 529.4 [M+1]⁺.

¹HNMR Spectrum: (500 MHz, CDCl₃) δ: 9.00 (t, J=6.0 Hz, 1H), 8.23 (s, 1H), 8.02 (d, J=1.8 Hz, 1H), 7.84 (d, J=8.0 Hz, 2H), 7.81-7.75 (m, 1H), 7.73 (d, J=7.5 Hz, 2H), 7.64 (d, J=3.1 Hz, 1H), 7.53 (d, J=1.7 Hz, 2H), 7.47 (t, J=7.7 Hz, 2H), 7.33 (d, J=3.2 Hz, 1H), 7.28 (d, J=7.9 Hz, 2H), 6.99 (d, J=8.0 Hz, 2H), 6.73 (d, J=3.1 Hz, 1H), 5.72 (s, 2H), 4.41 (d, J=5.8 Hz, 2H).

### EXAMPLE 3

### (R)-4-(1-(5-(4-fluorophenyl)-1-(4-(trifluoromethyl)benzyl)-1H-indole-7-carboxamido)ethyl)benzoic acid

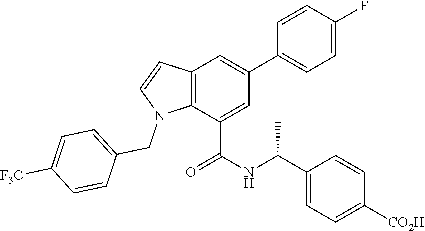 [SMILES: C[C@@H](NC(=O)c1cc(-c2ccc(F)cc2)cc2ccn(Cc3ccc(C(F)(F)F)cc3)c12)c1ccc(C(=O)O)cc1]

Starting with methyl (R)-4-(1-aminoethyl)benzoate and the product of Step 2 of Example 1 (30 mg), the title product was obtained as a white solid (19 mg) by following the same procedures as described in Step 3, Step 4 and Step 5 of Example 1. LCMS (ESI): 561.3 [M+1]⁺.

### EXAMPLE 4

### (R)-4-(1-(5-phenyl-1-(4-(trifluoromethyl)benzyl)-1H-indole-7-carboxamido)ethyl)benzoic acid

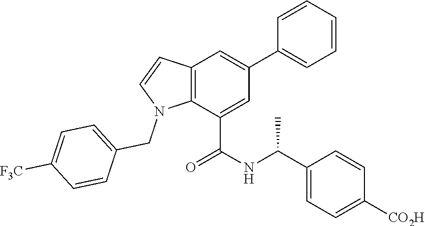 [SMILES: C[C@@H](NC(=O)c1cc(-c2ccccc2)cc2ccn(Cc3ccc(C(F)(F)F)cc3)c12)c1ccc(C(=O)O)cc1]

Starting with phenylboronic acid (55 mg) the title product was obtained as a white solid (19 mg) by following the same procedures described in Example 3. LCMS (ES+): 543.4 [M+1]*.

### EXAMPLE 5

### 4-(1-(5-(4-fluorophenyl)-1-(4-(trifluoromethyl)benzyl)-1H-indole-7-carboxamido)cyclopropyl)-benzoic acid

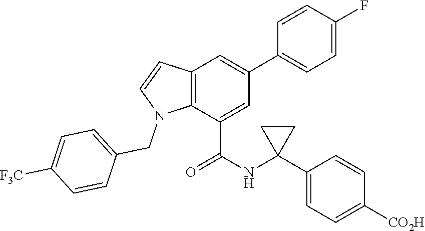 [SMILES: O=C(O)c1ccc(C2(NC(=O)c3cc(-c4ccc(F)cc4)cc4ccn(Cc5ccc(C(F)(F)F)cc5)c34)CC2)cc1]

Starting with methyl 4-(1-aminocyclopropyl)benzoate and 4-fluorophenyl)boronic acid (37 mg), the title product was obtained as a white solid (7.4 mg) by following the same procedure described in Step 3, Step 4 and Step 5 of Example 1. MS (ESI): 573.41 [M+1]⁺.

¹HNMR Spectrum: (500 MHz, DMSO-d₆) δ: 9.27 (s, 1H), 8.03 (d, J=1.8 Hz, 1H), 7.85-7.79 (m, 2H), 7.77 (d, J=8.2 Hz, 2H), 7.62-7.58 (m, 3H), 7.53 (d, J=3.1 Hz, 1H), 7.35-7.30 (m, 2H), 7.22 (d, J=8.2 Hz, 2H), 6.93 (d, J=7.9 Hz, 2H), 6.73 (d, J=3.1 Hz, 1H), 5.73 (s, 2H), 1.20 (q, J=5.1 Hz, 2H), 0.95 (q, J=5.0 Hz, 2H).

### EXAMPLE 6

### 4-(1-(5-phenyl-1-(4-(trifluoromethyl)benzyl)-1H-indole-7-carboxamido)cyclopropyl)benzoiacid

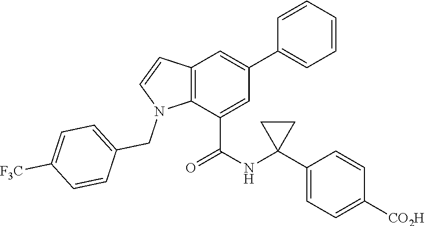 [SMILES: O=C(O)c1ccc(C2(NC(=O)c3cc(-c4ccccc4)cc4ccn(Cc5ccc(C(F)(F)F)cc5)c34)CC2)cc1]

Starting with phenylboronic acid (15 mg), the title product was obtained as a white solid (0.85 mg) by following the same procedures as described for Example 5. LCMS (ESI): 555.5 [M+1]⁺.

¹HNMR Spectrum: (500 MHz, DMSO-d₆) δ 12.77 (s, 1H), 9.29 (s, 1H), 8.09 (s, 2H), 8.06 (d, J=1.8 Hz, 1H), 7.78 (t, J=7.4 Hz, 4H), 7.64 (d, J=1.9 Hz, 1H), 7.60 (d, J=8.1 Hz, 2H), 7.53 (d, J=3.2 Hz, 1H), 7.50 (t, J=7.7 Hz, 2H), 7.36 (t, J=7.4 Hz, 1H), 7.23 (d, J=8.2 Hz, 2H), 6.95 (d, J=8.0 Hz, 2H), 6.75 (d, J=3.1 Hz, 1H), 5.74 (s, 2H), 1.25 (d, J=11.7 Hz, 2H), 0.86 (d, J=7.1 Hz, 2H).

### EXAMPLE 7

### 4-((5-(3-cyanophenyl)-1-(4-(trifluoromethyl)benzyl)-1H-indole-7-carboxamido)methyl)benzoic acid

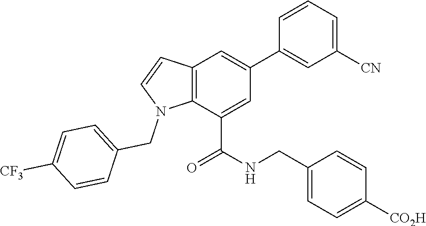 [SMILES: N#Cc1cccc(-c2cc(C(=O)NCc3ccc(C(=O)O)cc3)c3c(ccn3Cc3ccc(C(F)(F)F)cc3)c2)c1]

Starting with (3-cyanophenyl)boronic acid (15 mg), the title product was obtained as a white solid (10 mg) by following the same procedures as described in Example 1. LCMS (ESI): 554.3 [M+1]⁺. ¹H NMR (500 MHz, DMSO-d₆) δ 8.97 (d, J=6.1 Hz, 1H), 8.22 (s, 1H), 8.15-8.07 (m, 2H), 7.83 (d, J=8.0 Hz, 2H), 7.79 (d, J=7.7 Hz, 1H), 7.71-7.64 (m, 2H), 7.60 (d, J=1.9 Hz, 1H), 7.54 (d, J=8.1 Hz, 2H), 7.27 (d, J=7.8 Hz, 2H), 6.98 (d, J=8.0 Hz, 2H), 6.75 (d, J=3.1 Hz, 1H), 5.74 (s, 2H), 4.43 (d, J=5.8 Hz, 2H).

### EXAMPLE 8

### 4-((5-(3,4-difluorophenyl)-1-(4-(trifluoromethyl)benzyl)-1H-indole-7-carboxamido)methyl)benzoic acid

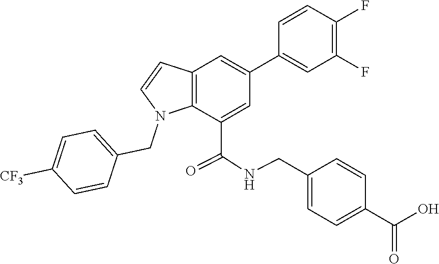 [SMILES: O=C(O)c1ccc(CNC(=O)c2cc(-c3ccc(F)c(F)c3)cc3ccn(Cc4ccc(C(F)(F)F)cc4)c23)cc1]

Starting with (3,4-difluorophenyl)boronic acid, the title product was obtained as a white solid by following the same procedure described in Example 1. MS (ESI): 565.5 [M+1]⁺.

¹HNMR Spectrum: (500 MHz, DMSO-d₆) δ 8.97 (t, J=5.9 Hz, 1H), 8.05 (d, J=1.9 Hz, 1H), 7.83 (dd, J=7.6, 5.7 Hz, 3H), 7.65 (d, J=3.1 Hz, 1H), 7.55-7.50 (m, 4H), 7.31-7.24 (m, 2H), 6.97 (d, J=8.0 Hz, 2H), 6.73 (d, J=3.1 Hz, 1H), 5.73 (s, 2H), 4.42 (d, J=5.8 Hz, 2H).

### EXAMPLE 9

### 4-((5-(3,5-difluorophenyl)-1-(4-(trifluoromethyl)benzyl)-1H-indole-7-carboxamido)methyl)benzoic acid

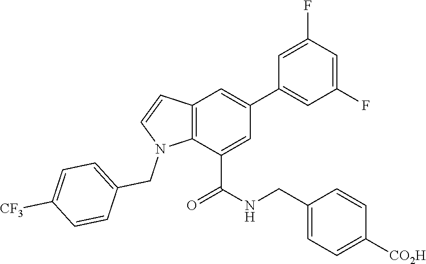 [SMILES: O=C(O)c1ccc(CNC(=O)c2cc(-c3cc(F)cc(F)c3)cc3ccn(Cc4ccc(C(F)(F)F)cc4)c23)cc1]

Starting with (3,5-difluorophenyl)boronic acid (15 mg), the title product was obtained as a white solid (10 mg) by following the same procedures in Example 1. LCMS (ESI): 565.4 [M+1]⁺. ¹HNMR Spectrum: (500 MHz, CDCl₃) δ: 12.77 (s, 1H), 8.97 (t, J=5.9 Hz, 1H), 8.13 (d, J=1.9 Hz, 1H), 7.84 (d, J=8.0 Hz, 2H), 7.67 (d, J=3.2 Hz, 1H), 7.59 (d, J=1.9 Hz, 1H), 7.52 (t, J=9.0 Hz, 4H), 7.28 (d, J=8.0 Hz, 2H), 7.18 (tt, J=9.2, 2.4 Hz, 1H), 6.97 (d, J=8.0 Hz, 2H), 6.75 (d, J=3.2 Hz, 1H), 5.74 (s, 2H), 4.44 (d, J=5.8 Hz, 2H).

### EXAMPLE 10

### 4-((1-(4-(trifluoromethyl)benzyl)-5-(3-(trifluoromethyl)phenyl)-1H-indole-7-carboxamido)methyl)benzoic acid

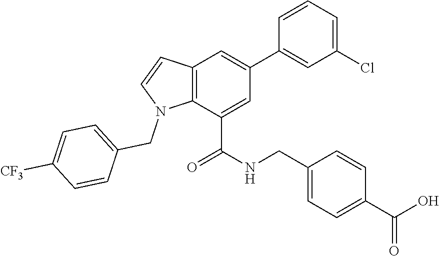 [SMILES: O=C(O)c1ccc(CNC(=O)c2cc(-c3cccc(Cl)c3)cc3ccn(Cc4ccc(C(F)(F)F)cc4)c23)cc1]

Starting with (3-(trifluoromethyl)phenyl)boronic acid, the title product was obtained as a white solid by following the same procedure described in Example 1. MS (ESI): 597.5 [M+1]⁺.

¹HNMR Spectrum: (500 MHz, DMSO-d₆) δ 12.84 (s, 1H), 9.02 (t, J=6.0 Hz, 1H), 8.14 (d, J=1.8 Hz, 1H), 8.09-8.00 (m, 2H), 7.83 (d, J=8.0 Hz, 2H), 7.72-7.68 (m, 2H), 7.66 (d, J=3.3 Hz, 1H), 7.59 (d, J=1.8 Hz, 1H), 7.54 (d, J=8.1 Hz, 2H), 7.29 (d, J=7.9 Hz, 2H), 6.98 (d, J=8.0 Hz, 2H), 6.77 (d, J=3.1 Hz, 1H), 5.74 (s, 2H), 4.44 (d, J=5.8 Hz, 2H).

### EXAMPLE 11

### 4-((5-(3-fluoro-5-(trifluoromethyl)phenyl)-1-(4-(trifluoromethyl)benzyl)-1H-indole-7-carboxamido)methyl)benzoic acid

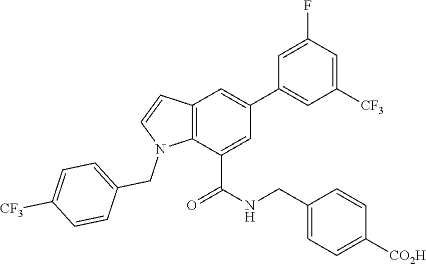 [SMILES: O=C(O)c1ccc(CNC(=O)c2cc(-c3cc(F)cc(C(F)(F)F)c3)cc3ccn(Cc4ccc(C(F)(F)F)cc4)c23)cc1]

Starting with (3-fluoro-5-(trifluoromethyl)phenyl)boronic acid (15 mg), the title product was obtained as a white solid (10 mg) by following the same procedures as described for Example 1. LCMS (ESI): 615.4 [M+1]⁺. ¹H NMR (500 MHz, DMSO-d₆) δ 12.84 (s, 1H), 9.00 (t, J=6.0 Hz, 1H), 8.20 (s, 1H), 7.96 (d, J=10.0 Hz, 2H), 7.83 (d, J=7.7 Hz, 2H), 7.68 (d, J=3.3 Hz, 1H), 7.63 (d, J=9.6 Hz, 2H), 7.53 (d, J=8.0 Hz, 2H), 7.28 (d, J=7.8 Hz, 2H), 6.97 (d, J=7.9 Hz, 2H), 6.77 (d, J=3.1 Hz, 1H), 5.75 (d, J=4.3 Hz, 2H), 4.45 (d, J=5.8 Hz, 2H).

### EXAMPLE 12

### (R)-4-(1-(5-(3-cyanophenyl)-1-(4-(trifluoromethyl)benzyl)-1H-indole-7-carboxamido)ethyl) benzoic acid

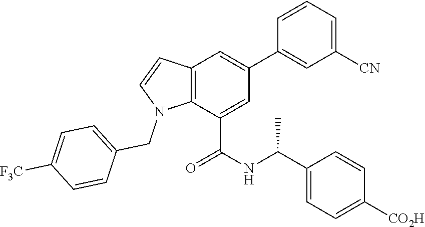 [SMILES: C[C@@H](NC(=O)c1cc(-c2cccc(C#N)c2)cc2ccn(Cc3ccc(C(F)(F)F)cc3)c12)c1ccc(C(=O)O)cc1]

Starting with (3-cyanophenyl) boronic acid (39 mg), the title product was obtained as a white solid (11 mg) by following the same procedures as described in Example 3. LCMS (ESI): 568.4 [M+1]*.

### EXAMPLE 13

### (R)-4-(1-(5-(3,4-difluorophenyl)-1-(4-(trifluoromethyl)benzyl)-1H-indole-7-carboxamido)ethyl)benzoic acid

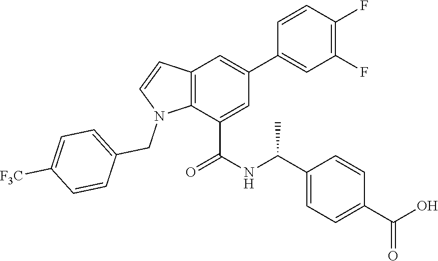 [SMILES: C[C@@H](NC(=O)c1cc(-c2ccc(F)c(F)c2)cc2ccn(Cc3ccc(C(F)(F)F)cc3)c12)c1ccc(C(=O)O)cc1]

Starting with (3,4-difluorophenyl)boronic acid (15 mg) and the product of Step 3 of Example 1(20 mg), the title product was obtained as a white solid (25 mg) by following the same procedures as described in Example 3. LCMS (ES+): 579.4 [M+1]⁺.

### EXAMPLE 14

### (R)-4-(1-(5-(3,5-difluorophenyl)-1-(4-(trifluoromethyl)benzyl)-1H-indole-7-carboxamido)ethyl) benzoic acid

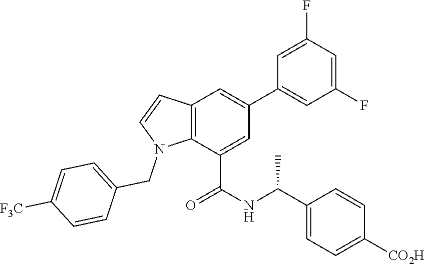 [SMILES: C[C@@H](NC(=O)c1cc(-c2cc(F)cc(F)c2)cc2ccn(Cc3ccc(C(F)(F)F)cc3)c12)c1ccc(C(=O)O)cc1]

Starting with (3,5-difluorophenyl) boronic acid (42 mg), the title product was obtained as a white solid (15 mg) by following the same procedure described in Example 3. LCMS (ESI): 579.4 [M+1]*.

### EXAMPLE 15

### (R)-4-(1-(1-(4-(trifluoromethyl)benzyl)-5-(3-(trifluoromethyl)phenyl)-1H-indole-7-carboxamido)ethyl) benzoic acid

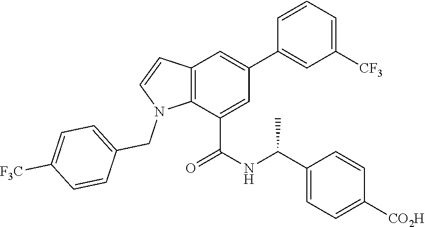 [SMILES: C[C@@H](NC(=O)c1cc(-c2cccc(C(F)(F)F)c2)cc2ccn(Cc3ccc(C(F)(F)F)cc3)c12)c1ccc(C(=O)O)cc1]

Starting with (3-(trifluoromethyl)phenyl) boronic acid (51 mg), the title product was obtained as a white solid (12 mg) by following the same procedures as described in Example 3. LCMS (ESI): 611.4 [M+1]

### EXAMPLE 16

### (R)-4-(1-(5-(3-fluoro-5-(trifluoromethyl)phenyl)-1-(4-(trifluoromethyl)benzyl)-1H-indole-7-carboxamido)ethyl)benzoic acid

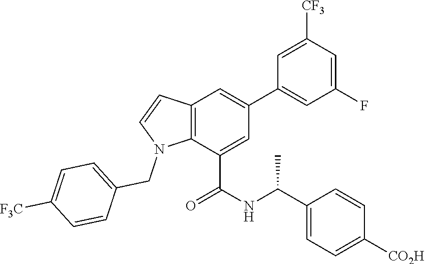 [SMILES: C[C@@H](NC(=O)c1cc(-c2cc(F)cc(C(F)(F)F)c2)cc2ccn(Cc3ccc(C(F)(F)F)cc3)c12)c1ccc(C(=O)O)cc1]

Starting with (3-fluoro-5-(trifluoromethyl)phenyl)boronic acid (15 mg) and the product of Step 3 of Example 1 (20 mg), the title product was obtained as a white solid (25 mg) by following the same procedures as described in Step 4 and Step 5 of Example 3. LCMS (ES+): 629.2 [M+1]⁺.

### EXAMPLE 17

### 4-(1-(5-(3-cyanophenyl)-1-(4-(trifluoromethyl)benzyl)-1H-indole-7-carboxamido)cyclopropyl)benzoic acid

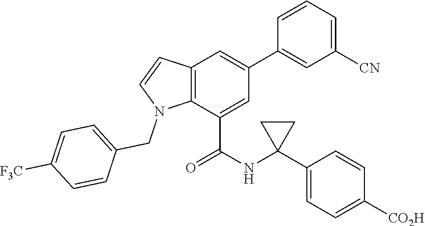 [SMILES: N#Cc1cccc(-c2cc(C(=O)NC3(c4ccc(C(=O)O)cc4)CC3)c3c(ccn3Cc3ccc(C(F)(F)F)cc3)c2)c1]

Starting with (3-cyanophenyl)boronic acid (15 mg), the title product was obtained as a white solid (0.85 mg) by following the same procedures as described in Example 5. LCMS (ESI): 580.5 [M+1]⁺.

### EXAMPLE 18

### 4-(1-(5-(3,4-difluorophenyl)-1-(4-(trifluoromethyl)benzyl)-1H-indole-7-carboxamido)cyclopropyl)benzoic acid

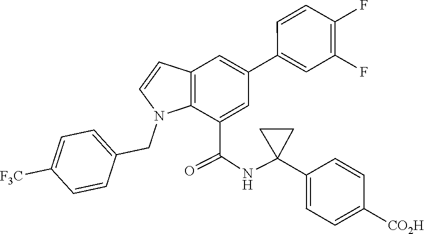 [SMILES: O=C(O)c1ccc(C2(NC(=O)c3cc(-c4ccc(F)c(F)c4)cc4ccn(Cc5ccc(C(F)(F)F)cc5)c34)CC2)cc1]

Starting with (3,4-difluorophenyl)boronic acid (15 mg), the title product was obtained as a white solid (0.85 mg) by following the same procedures as described in Example 5. LCMS (ESI): 591.5 [M+1]⁺.

### EXAMPLE 19

### 4-(1-(5-(3,5-difluorophenyl)-1-(4-(trifluoromethyl)benzyl)-1H-indole-7-carboxamido)cyclopropyl)benzoic acid

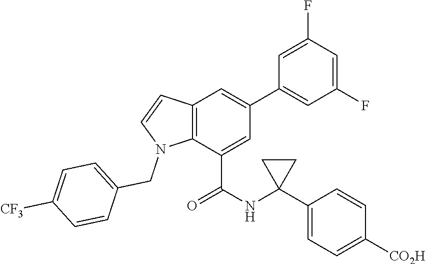 [SMILES: O=C(O)c1ccc(C2(NC(=O)c3cc(-c4cc(F)cc(F)c4)cc4ccn(Cc5ccc(C(F)(F)F)cc5)c34)CC2)cc1]

Starting with (3,5-difluorophenyl)boronic acid (15 mg), the title product was obtained as a white solid (10 mg) by following the same procedures as described in Example 5. LCMS (ESI): 591.4 [M+1]⁺. ¹H NMR (500 MHz, DMSO-d₆) δ 12.77 (s, 1H), 9.26 (s, 1H), 8.17 (d, J=1.8 Hz, 1H), 7.77 (d, J=8.4 Hz, 2H), 7.70 (d, J=1.9 Hz, 1H), 7.62-7.55 (m, 6H), 7.23 (d, J=8.4 Hz, 3H), 6.93 (d, J=8.0 Hz, 2H), 6.75 (d, J=3.1 Hz, 1H), 5.75 (s, 2H), 1.22 (t, J=3.6 Hz, 2H), 0.99 (q, J=5.0 Hz, 2H).

### EXAMPLE 20

### 4-(1-(1-(4-(trifluoromethyl)benzyl)-5-(3-(trifluoromethyl)phenyl)-1H-indole-7-carboxamido)cyclopropyl)benzoic acid

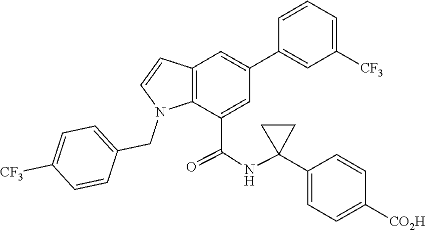 [SMILES: O=C(O)c1ccc(C2(NC(=O)c3cc(-c4cccc(C(F)(F)F)c4)cc4ccn(Cc5ccc(C(F)(F)F)cc5)c34)CC2)cc1]

Starting with (3-(trifluoromethyl)phenyl)boronic acid (15 mg), the title product was obtained as a white solid (10 mg) by following the same procedures as described in Example 5. LCMS (ESI): 623.4 [M+1]⁺. ₁H NMR (500 MHz, DMSO-d₆) δ 12.78 (s, 1H), 9.30 (s, 1H), 8.17 (d, J=1.8 Hz, 1H), 8.12 (d, J=8.2 Hz, 2H), 7.79-7.68 (m, 5H), 7.60 (d, J=8.2 Hz, 2H), 7.57 (d, J=3.2 Hz, 1H), 7.23 (d, J=8.3 Hz, 2H), 6.94 (d, J=8.0 Hz, 2H), 6.77 (d, J=3.1 Hz, 1H), 5.75 (s, 2H), 1.23-1.20 (m, 2H), 0.97 (q, J=5.1 Hz, 2H).

### EXAMPLE 21

### 4-(1-(5-(3-fluoro-5-(trifluoromethyl)phenyl)-1-(4-(trifluoromethyl)benzyl)-1H-indole-7-carboxamido)cyclopropyl)benzoic acid

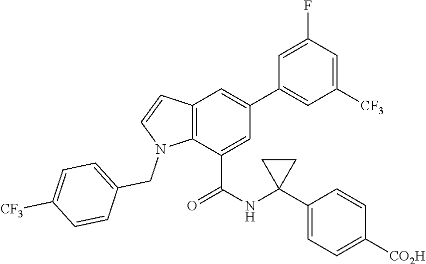 [SMILES: O=C(O)c1ccc(C2(NC(=O)c3cc(-c4cc(F)cc(C(F)(F)F)c4)cc4ccn(Cc5ccc(C(F)(F)F)cc5)c34)CC2)cc1]

Starting with (3-fluoro-5-(trifluoromethyl)phenyl)boronic acid (15 mg), the title product was obtained as a white solid (10 mg) by following the same procedures as described in Example 5. LCMS (ESI): 641.4 [M+1]⁺.

¹H NMR (500 MHz, DMSO-d₆) δ 11.16 (s, 1H), 9.20 (s, 1H), 8.21 (d, J=1.8 Hz, 1H), 8.04-7.98 (m, 2H), 7.75 (d, J=7.9 Hz, 2H), 7.70-7.64 (m, 2H), 7.61-7.58 (m, 3H), 7.12 (d, J=8.0 Hz, 2H), 6.93 (d, J=7.9 Hz, 2H), 6.77 (d, J=3.1 Hz, 1H), 5.73 (s, 2H), 1.15 (q, J=5.0 Hz, 2H), 0.91 (t, J=3.6 Hz, 2H).

### EXAMPLE 22

### 4-((4-(3-cyanophenyl)-1-(4-(trifluoromethyl)benzyl)-1H-indole-7-carboxamido)methyl)benzoic acid

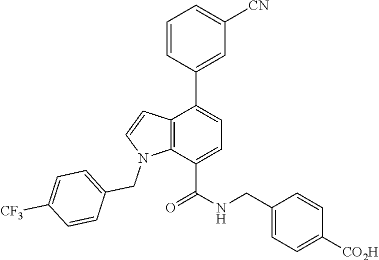 [SMILES: N#Cc1cccc(-c2ccc(C(=O)NCc3ccc(C(=O)O)cc3)c3c2ccn3Cc2ccc(C(F)(F)F)cc2)c1]

Starting with methyl 4-bromo-1H-indole-7-carboxylate and (3-cyanophenyl)boronic acid, the title product was obtained as a white solid (2.8 mg) by following the same procedure described in Step 1, Step 2, Step 3, Step 4 and Step 5 of Example 1. LCMS (ESI): 565.4 [M+1]⁺.

### EXAMPLE 23

### 4-((4-(3,4-difluorophenyl)-1-(4-(trifluoromethyl)benzyl)-1H-indole-7-carboxamido)methyl) benzoic acid

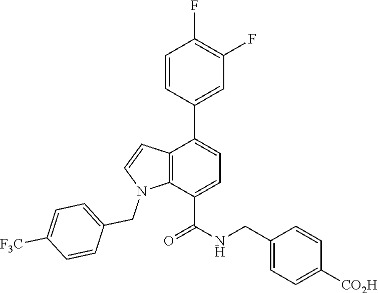 [SMILES: O=C(O)c1ccc(CNC(=O)c2ccc(-c3ccc(F)c(F)c3)c3ccn(Cc4ccc(C(F)(F)F)cc4)c23)cc1]

Starting with (4-fluorophenyl)boronic acid (30 mg), the title product was obtained as a white solid (28.8 mg) by following the same procedures as described in Example 22. LCMS (ESI): 565.4 [M+1]*.

### EXAMPLE 24

### 4-((1-(4-(trifluoromethyl)benzyl)-4-(3-(trifluoromethyl)phenyl)-1H-indole-7-carboxamido)-methyl)benzoic acid

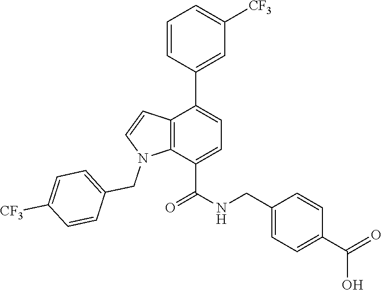 [SMILES: O=C(O)c1ccc(CNC(=O)c2ccc(-c3cccc(C(F)(F)F)c3)c3ccn(Cc4ccc(C(F)(F)F)cc4)c23)cc1]

Starting with (3-(trifluoromethyl)phenyl)boronic acid (95 mg), the title product was obtained as a white solid (28.8 mg) by following the same procedures as described in Example 22.

### LCMS (ESI): 597.41 [M+1]⁺.

### EXAMPLE 25

### 4-((4-(4-fluorophenyl)-1-(4-(trifluoromethyl)benzyl)-1H-indole-7-carboxamido)methyl)benzoic acid

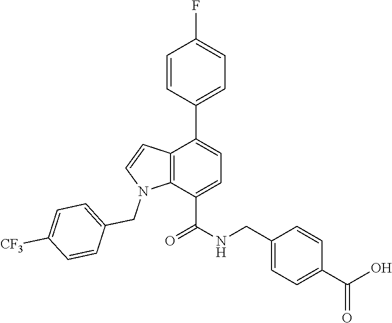 [SMILES: O=C(O)c1ccc(CNC(=O)c2ccc(-c3ccc(F)cc3)c3ccn(Cc4ccc(C(F)(F)F)cc4)c23)cc1]

Starting with (4-fluorophenyl)boronic acid (15 mg), the title product was obtained as a white solid (0.85 mg) by following the same procedures as described in Example 22. LCMS (ESI): 547.5 [M+1]⁺.

### EXAMPLE 26

### 4-((4-phenyl-1-(4-(trifluoromethyl)benzyl)-1H-indole-7-carboxamido)methyl)benzoic acid

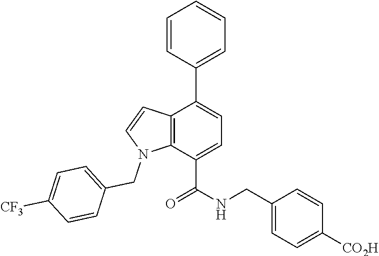 [SMILES: O=C(O)c1ccc(CNC(=O)c2ccc(-c3ccccc3)c3ccn(Cc4ccc(C(F)(F)F)cc4)c23)cc1]

Starting with phenylboronic acid, the title product was obtained as a white solid (14.6 mg) by following the same procedures as described in Example 22. LCMS (ESI): 529.41 [M+1]*.

### EXAMPLE 27

### 4-((4-(3,5-difluorophenyl)-1-(4-(trifluoromethyl)benzyl)-1H-indole-7-carboxamido)methyl) benzoic acid

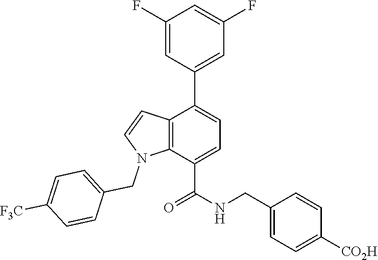 [SMILES: O=C(O)c1ccc(CNC(=O)c2ccc(-c3cc(F)cc(F)c3)c3ccn(Cc4ccc(C(F)(F)F)cc4)c23)cc1]

Starting with (3,5-difluorophenyl) boronic acid (43 mg), the title product was obtained as a white solid (21 mg) by following the same procedures as described in Example 22. LCMS (ESI): 565.4 [M+1]*.

### EXAMPLE 28

### 4-((4-(3-fluoro-5-(trifluoromethyl)phenyl)-1-(4-(trifluoromethyl)benzyl)-1H-indole-7-carboxamido)methyl)benzoic acid

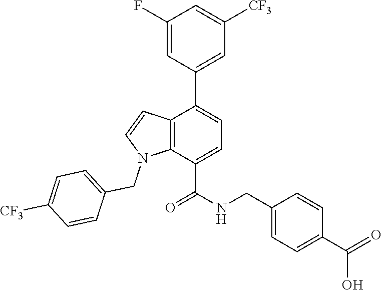 [SMILES: O=C(O)c1ccc(CNC(=O)c2ccc(-c3cc(F)cc(C(F)(F)F)c3)c3ccn(Cc4ccc(C(F)(F)F)cc4)c23)cc1]

Starting with (3-fluoro-5-(trifluoromethyl)phenyl)boronic acid (10⁴ mg), the title product was obtained as a white solid (37.3 mg) by following the same procedures as described in Example 22. LCMS (ESI): 615.35 [M+1]⁺.

### EXAMPLE 29

### (R)-4-(1-(4-phenyl-1-(4-(trifluoromethyl)benzyl)-1H-indole-7-carboxamido)ethyl) benzoic acid

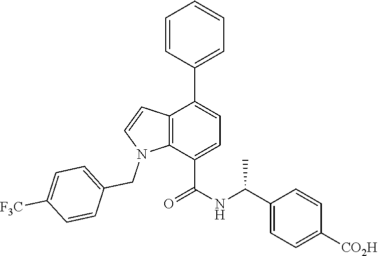 [SMILES: C[C@@H](NC(=O)c1ccc(-c2ccccc2)c2ccn(Cc3ccc(C(F)(F)F)cc3)c12)c1ccc(C(=O)O)cc1]

Starting with methyl 4-bromo-1H-indole-7-carboxylate, methyl (R)-4-(1-aminoethyl)benzoate and phenylboronic acid, the title product was obtained as a white solid by following the same procedure described in Step 1, Step 2, Step 3, Step 4 and Step 5 of Example 1. LCMS (ESI): 543.3 [M+1]⁺.

### EXAMPLE 30

### (R)-4-(1-(4-(3-cyanophenyl)-1-(4-(trifluoromethyl)benzyl)-1H-indole-7-carboxamido)ethyl) benzoic acid

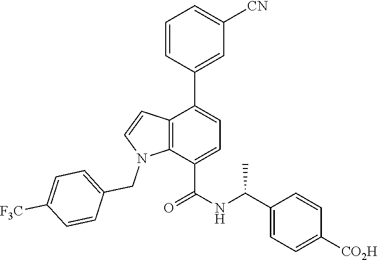 [SMILES: C[C@@H](NC(=O)c1ccc(-c2cccc(C#N)c2)c2ccn(Cc3ccc(C(F)(F)F)cc3)c12)c1ccc(C(=O)O)cc1]

Starting with (3-cyanophenyl) boronic acid (39 mg), the title product was obtained as a white solid (23 mg) by following the same procedures as described in Example 29. LCMS (ESI): 568.4 [M+1]*.

### EXAMPLE 31

### (R)-4-(1-(4-(3,4-difluorophenyl)-1-(4-(trifluoromethyl)benzyl)-1H-indole-7-carboxamido)ethyl)benzoic acid

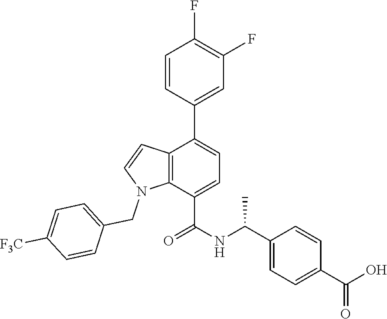 [SMILES: C[C@@H](NC(=O)c1ccc(-c2ccc(F)c(F)c2)c2ccn(Cc3ccc(C(F)(F)F)cc3)c12)c1ccc(C(=O)O)cc1]

Starting with (3,4-difluorophenyl)boronic acid (15 mg) and the product of Step 3 of Example 1(20 mg), the title product was obtained as a white solid (25 mg) by following the same procedures as described in Example 29. LCMS (ESI): 579.4 [M+1]⁺.

### EXAMPLE 32

### (R)-4-(1-(4-(3,5-difluorophenyl)-1-(4-(trifluoromethyl)benzyl)-1H-indole-7-carboxamido)ethyl) benzoic acid

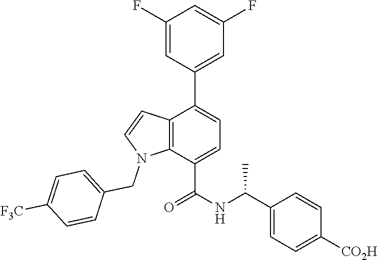 [SMILES: C[C@@H](NC(=O)c1ccc(-c2cc(F)cc(F)c2)c2ccn(Cc3ccc(C(F)(F)F)cc3)c12)c1ccc(C(=O)O)cc1]

Starting with (3,5-difluorophenyl) boronic acid (15 mg), the title product was obtained as a white solid (21 mg) by following the same procedures as described in Example 29. LCMS (ESI): 579.3 [M+1]*.

### EXAMPLE 33

### (R)-4-(1-(1-(4-(trifluoromethyl)benzyl)-4-(3-(trifluoromethyl)phenyl)-1H-indole-7-carboxamido)ethyl) benzoic acid

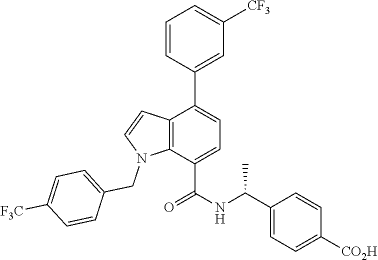 [SMILES: C[C@@H](NC(=O)c1ccc(-c2cccc(C(F)(F)F)c2)c2ccn(Cc3ccc(C(F)(F)F)cc3)c12)c1ccc(C(=O)O)cc1]

Starting with (3-(trifluoromethyl)phenyl) boronic acid (51 mg), the title product was obtained as a white solid (23 mg) by following the same procedures as described in Example 29. LCMS (ESI): 611.4 [M+1]*.

### EXAMPLE 34

### (R)-4-(1-(4-(3-fluoro-5-(trifluoromethyl)phenyl)-1-(4-(trifluoromethyl)benzyl)-1H-indole-7-carboxamido)ethyl)benzoic acid

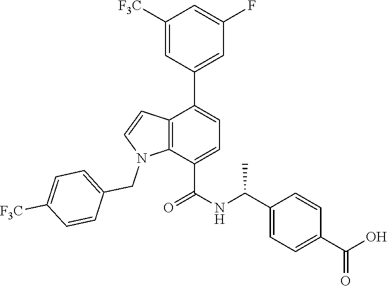 [SMILES: C[C@@H](NC(=O)c1ccc(-c2cc(F)cc(C(F)(F)F)c2)c2ccn(Cc3ccc(C(F)(F)F)cc3)c12)c1ccc(C(=O)O)cc1]

Starting with (3-fluoro-5-(trifluoromethyl)phenyl)boronic acid (15 mg) and the product of Step 3 of Example 1 (20 mg), the title product was obtained as a white solid (25 mg) by following the same procedures as described in Example 29. LCMS (ESI): 629.5 [M+1]⁺.

### EXAMPLE 35

### (R)-4-(1-(4-(4-fluorophenyl)-1-(4-(trifluoromethyl)benzyl)-1H-indole-7-carboxamido)ethyl)benzoic acid

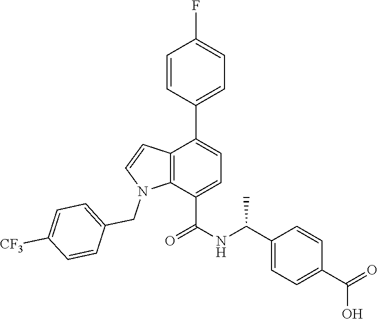 [SMILES: C[C@@H](NC(=O)c1ccc(-c2ccc(F)cc2)c2ccn(Cc3ccc(C(F)(F)F)cc3)c12)c1ccc(C(=O)O)cc1]

Starting with (4-fluorophenyl)boronic acid (63 mg), the title product was obtained as a white solid (7.5 mg) by following the same procedures as described in Example 29.

### LCMS (ESI): 561.32 [M+1]⁺.

### EXAMPLE 36

### 4-(1-(4-phenyl-1-(4-(trifluoromethyl)benzyl)-1H-indole-7-carboxamido)cyclopropyl)benzoic acid

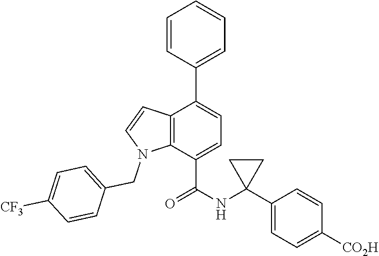 [SMILES: O=C(O)c1ccc(C2(NC(=O)c3ccc(-c4ccccc4)c4ccn(Cc5ccc(C(F)(F)F)cc5)c34)CC2)cc1]

Starting with methyl 4-bromo-1H-indole-7-carboxylate, methyl 4-(1-aminocyclopropyl)benzoate and phenylboronic acid, the title product was obtained as a white solid by following the same procedure described in Step 1, Step 2, Step 3, Step 4 and Step 5 of Example 1. LCMS (ESI): 555.3 [M+1]⁺.

### EXAMPLE 37

### 4-(1-(4-(3-cyanophenyl)-1-(4-(trifluoromethyl)benzyl)-1H-indole-7-carboxamido)cyclopropyl)benzoic acid

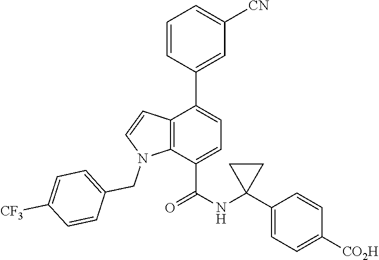 [SMILES: N#Cc1cccc(-c2ccc(C(=O)NC3(c4ccc(C(=O)O)cc4)CC3)c3c2ccn3Cc2ccc(C(F)(F)F)cc2)c1]

Starting with (3-cyanophenyl)boronic acid (15 mg), the title product was obtained as a white solid (7.8 mg) by following the same procedures as described in Example 36. LCMS (ESI): 580.3 [M+1]⁺.

### EXAMPLE 38

### 4-(1-(4-(3,4-difluorophenyl)-1-(4-(trifluoromethyl)benzyl)-1H-indole-7-carboxamido)cyclopropyl)benzoic acid

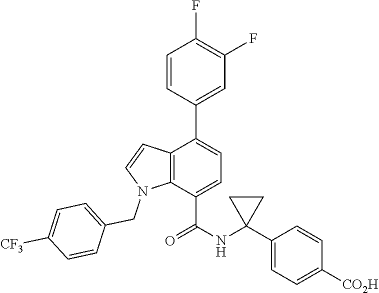 [SMILES: O=C(O)c1ccc(C2(NC(=O)c3ccc(-c4ccc(F)c(F)c4)c4ccn(Cc5ccc(C(F)(F)F)cc5)c34)CC2)cc1]

Starting with (3,4-difluorophenyl)boronic acid, the title product was obtained as a white solid by following the same procedure described in Example 36. LCMS (ESI): 591.5 [M+1]⁺.

### EXAMPLE 39

### 4-(1-(4-(3,5-difluorophenyl)-1-(4-(trifluoromethyl)benzyl)-1H-indole-7-carboxamido)cyclopropyl)benzoic acid

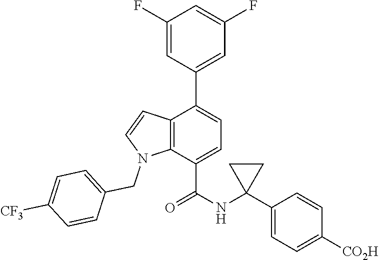 [SMILES: O=C(O)c1ccc(C2(NC(=O)c3ccc(-c4cc(F)cc(F)c4)c4ccn(Cc5ccc(C(F)(F)F)cc5)c34)CC2)cc1]

Starting with (3,5-difluorophenyl)boronic acid (15 mg), the title product was obtained as a white solid (10 mg) by following the same procedures as described in Example 36. LCMS (ESI): 591.2 [M+1]⁺.

### EXAMPLE 40

### 4-(1-(1-(4-(trifluoromethyl)benzyl)-4-(3-(trifluoromethyl)phenyl)-1H-indole-7-carboxamido)cyclopropyl)benzoic acid

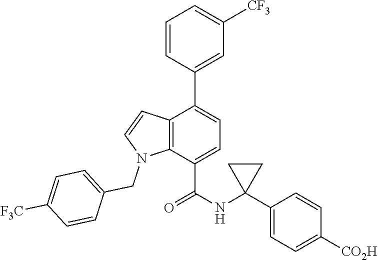 [SMILES: O=C(O)c1ccc(C2(NC(=O)c3ccc(-c4cccc(C(F)(F)F)c4)c4ccn(Cc5ccc(C(F)(F)F)cc5)c34)CC2)cc1]

Starting with (3-(trifluoromethyl)phenyl)boronic acid, the title product was obtained as a white solid by following the same procedure described in Step 3, Step 4 and Step 5 of Example 36. LCMS (ESI): 623.5 [M+1]⁺.

### EXAMPLE 41

### 4-(1-(4-(3-fluoro-5-(trifluoromethyl)phenyl)-1-(4-(trifluoromethyl)benzyl)-1H-indole-7-carboxamido)cyclopropyl)benzoic acid

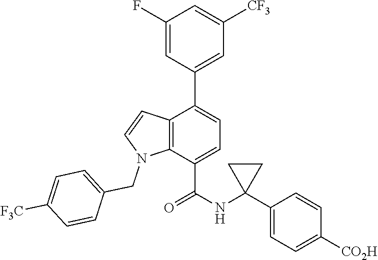 [SMILES: O=C(O)c1ccc(C2(NC(=O)c3ccc(-c4cc(F)cc(C(F)(F)F)c4)c4ccn(Cc5ccc(C(F)(F)F)cc5)c34)CC2)cc1]

Starting with (3-fluoro-5-(trifluoromethyl)phenyl)boronic acid, the title product was obtained as a white solid by following the same procedure described in Example 36. LCMS (ESI): 641.5 [M+1]⁺.

### EXAMPLE 42

### 4-(1-(4-(4-fluorophenyl)-1-(4-(trifluoromethyl)benzyl)-1H-indole-7-carboxamido)cyclopropyl)-benzoic acid

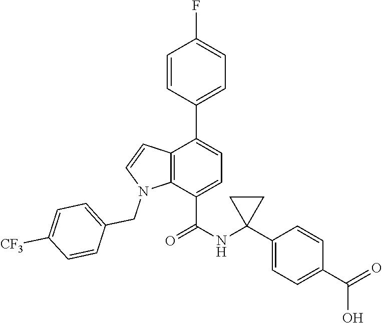 [SMILES: O=C(O)c1ccc(C2(NC(=O)c3ccc(-c4ccc(F)cc4)c4ccn(Cc5ccc(C(F)(F)F)cc5)c34)CC2)cc1]

Starting with (4-fluorophenyl)boronic acid (61.3 mg), the title product was obtained as a white solid (13.7 mg) by following the same procedures as described for Example 36. LCMS (ESI): 573.35 [M+1]⁺.

### EXAMPLE 43

### 4-((5-(phenylamino)-1-(4-(trifluoromethyl)benzyl)-1H-indole-7-carboxamido)methyl)benzoic acid

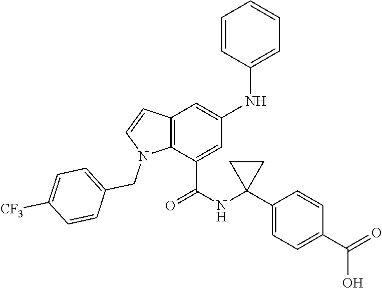 [SMILES: O=C(O)c1ccc(C2(NC(=O)c3cc(Nc4ccccc4)cc4ccn(Cc5ccc(C(F)(F)F)cc5)c34)CC2)cc1]

### Step 1 methyl 4-(1-(5-(phenylamino)-1-(4-(trifluoromethyl)benzyl)-1H-indole-7-carboxamido)cyclopropyl)benzoate

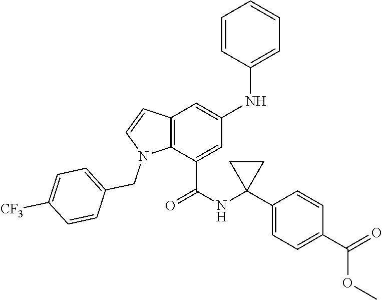 [SMILES: COC(=O)c1ccc(C2(NC(=O)c3cc(Nc4ccccc4)cc4ccn(Cc5ccc(C(F)(F)F)cc5)c34)CC2)cc1]

A mixture of the methyl 4-(1-(5-bromo-1-(4-(trifluoromethyl)benzyl)-1H-indole-7-carboxamido)cyclopropyl)benzoate (30 mg, 0.06 mmol), aniline (30 mg, 0.36 mmol), Xantphos (6.2 mg, 0.012 mmol), Pd₂(dba)₃ (3.2 mg, 0.006 mmol), Cs₂CO₃ (60 mg, 0.18 mmol) in 1,4-dioxane (5 ml) was stirred for 16 h at 120° C. under nitrogen. The reaction mixture was concentrated and purified by silica gel column chromatography (PE/EA=5:1) to afford the title compound as a yellow solid (2.0 mg). LCMS (ESI): 584.3 [M+1]⁺.

### Step 2 4-(1-(5-(phenylamino)-1-(4-(trifluoromethyl)benzyl)-1H-indole-7-carboxamido)cyclopropyl)benzoic acid

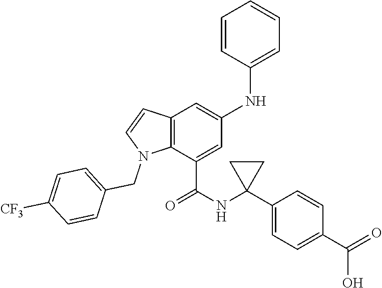 [SMILES: O=C(O)c1ccc(C2(NC(=O)c3cc(Nc4ccccc4)cc4ccn(Cc5ccc(C(F)(F)F)cc5)c34)CC2)cc1]

The title product was obtained as a white solid by following the same procedures as described in Step 5 of Example 1. LCMS (ESI): 570.3 [M+1]⁺.

### EXAMPLE 44

### 4-(1-(4-(phenylamino)-1-(4-(trifluoromethyl)benzyl)-1H-indole-7-carboxamido)cyclopropyl)benzoic acid

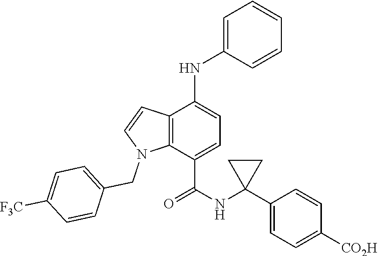 [SMILES: O=C(O)c1ccc(C2(NC(=O)c3ccc(Nc4ccccc4)c4ccn(Cc5ccc(C(F)(F)F)cc5)c34)CC2)cc1]

Starting with methyl 4-(1-aminocyclopropyl)benzoate, 4-bromo-1-(4-(trifluoromethyl)benzyl)-1H-indole-7-carboxylic acid and aniline (65 mg), the title product was obtained as a white solid(22 mg) by following the same procedure described in Example 43. LCMS (ES+): 570.4 [M+1]⁺.

¹HNMR Spectrum: (500 MHz, DMSO-d₆) δ: 8.93 (s, 1H), 8.31 (s, 1H), 7.74 (d, J=8.2 Hz, 2H), 7.59 (d, J=8.1 Hz, 2H), 7.39-7.35 (m, 1H), 7.34-7.25 (m, 4H), 7.16 (d, J=8.2 Hz, 2H), 6.96-6.86 (m, 5H), 5.74 (s, 2H), 1.20-1.15 (m, 2H), 0.98-0.94 (m, 2H).

### EXAMPLE 45

### 4-((5-(methyl(phenyl)amino)-1-(4-(trifluoromethyl)benzyl)-1H-indole-7-carboxamido)methyl)benzoic acid

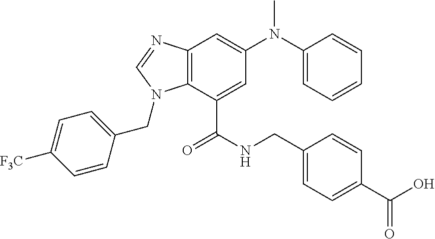 [SMILES: CN(c1ccccc1)c1cc(C(=O)NCc2ccc(C(=O)O)cc2)c2c(c1)ncn2Cc1ccc(C(F)(F)F)cc1]

Starting with N-methylaniline (15 mg) and the product of Step 3 of Example 1 (20 mg), the title product was obtained as a white solid (5 mg) by following the same procedures as described in Example 43. LCMS (ES+): 558.3 [M+1]⁺.

¹HNMR Spectrum: (500 MHz, DMSO-d₆) δ: 12.79 (s, 1H), 8.89 (t, J=6.0 Hz, 1H), 7.87-7.77 (m, 2H), 7.58 (d, J=3.2 Hz, 1H), 7.55 (d, J=8.0 Hz, 2H), 7.51 (d, J=2.1 Hz, 1H), 7.25 (d, J=8.0 Hz, 2H), 7.20-7.13 (m, 2H), 7.06 (d, J=2.1 Hz, 1H), 7.01 (d, J=8.0 Hz, 2H), 6.77 (d, J=8.1 Hz, 2H), 6.73 (t, J=7.2 Hz, 1H), 6.61 (d, J=3.2 Hz, 1H), 5.66 (s, 2H), 4.35 (d, J=5.7 Hz, 2H), 3.28 (s, 3H).

### EXAMPLE 46

### 4-(1-(4-(methyl(phenyl)amino)-1-(4-(trifluoromethyl)benzyl)-1H-indole-7-carboxamido)cyclopropyl)benzoic acid

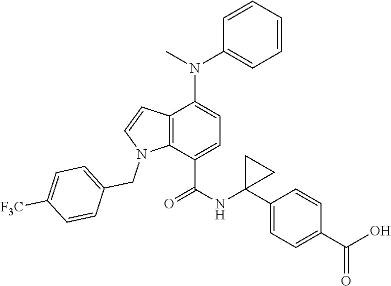 [SMILES: CN(c1ccccc1)c1ccc(C(=O)NC2(c3ccc(C(=O)O)cc3)CC2)c2c1ccn2Cc1ccc(C(F)(F)F)cc1]

Starting with N-methylaniline (15 mg) and the product of Step 3 of Example 1 (20 mg), the title product was obtained as a white solid (25 mg) by following the same procedures as described in Example 43. LCMS (ES+): 584.5 [M+1]⁺.

¹HNMR Spectrum: (500 MHz, DMSO-d₆) δ: 12.79 (s, 1H), 9.13 (s, 1H), 7.77 (d, J=8.4 Hz, 2H), 7.60 (d, J=8.1 Hz, 2H), 7.42 (d, J=7.9 Hz, 1H), 7.33 (d, J=3.2 Hz, 1H), 7.21 (dt, J=7.8, 3.6 Hz, 4H), 6.95 (dd, J=7.9, 2.0 Hz, 3H), 6.85-6.82 (m, 2H), 6.06 (d, J=3.3 Hz, 1H), 5.69 (s, 2H), 5.33 (t, J=5.0 Hz, 1H), 3.41 (s, 3H), 0.96-0.83 (m, 4H).

### EXAMPLE 47

### 4-((5-(phenylamino)-1-(4-(trifluoromethyl)benzyl)-1H-indole-7-carboxamido)methyl)benzoic acid

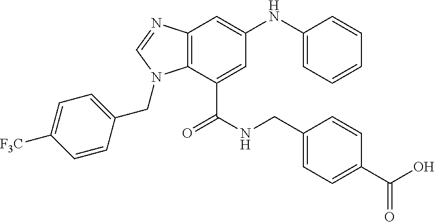 [SMILES: O=C(O)c1ccc(CNC(=O)c2cc(Nc3ccccc3)cc3ncn(Cc4ccc(C(F)(F)F)cc4)c23)cc1]

Starting with aniline (15 mg) and the product of Step 3 of Example 1 (20 mg), the title product was obtained as a white solid (17 mg) by following the same procedures as described in Example 43. LCMS (ES+): 544.4 [M+1]⁺.

¹HNMR Spectrum: (500 MHz, DMSO-d₆) δ: 12.87 (s, 1H), 8.90 (t, J=6.0 Hz, 1H), 7.97 (s, 1H), 7.83 (d, J=8.1 Hz, 2H), 7.56 (d, J=8.1 Hz, 2H), 7.51 (d, J=3.2 Hz, 1H), 7.44 (d, J=2.1 Hz, 1H), 7.29 (d, J=8.0 Hz, 2H), 7.18 (t, J=7.8 Hz, 2H), 7.07-6.99 (m, 4H), 6.73 (t, J=7.3 Hz, 1H), 6.54 (d, J=3.1 Hz, 1H), 5.60 (s, 2H), 4.34 (d, J=6.0 Hz, 2H).

### EXAMPLE 48

### 4-(1-(4-(methyl(phenyl)amino)-1-(4-(trifluoromethyl)benzyl)-1H-indole-7-carboxamido)cyclopropyl)benzoic acid

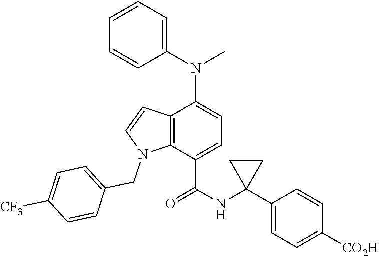 [SMILES: CN(c1ccccc1)c1ccc(C(=O)NC2(c3ccc(C(=O)O)cc3)CC2)c2c1ccn2Cc1ccc(C(F)(F)F)cc1]

Starting with N-methylaniline (15 mg), the title product was obtained as a white solid (0.85 mg) by following the same procedures as described in Example 43. LCMS (ESI): 598.6 [M+1]⁺.

### EXAMPLE 49

### 4-(1-(5-phenoxy-1-(4-(trifluoromethyl)benzyl)-1H-indole-7-carboxamido)cyclopropyl)benzoic acid

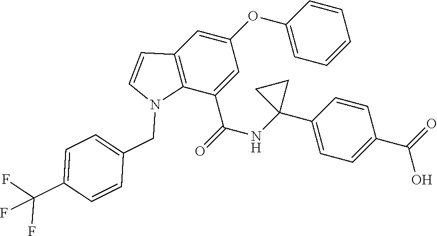 [SMILES: O=C(O)c1ccc(C2(NC(=O)c3cc(Oc4ccccc4)cc4ccn(Cc5ccc(C(F)(F)F)cc5)c34)CC2)cc1]

A mixture of the methyl 4-(1-(5-bromo-1-(4-(trifluoromethyl)benzyl)-1H-indole-7-carboxamido)cyclopropyl)benzoate (30 mg, 0.06 mmol), phenol (30 mg, 0.36 mmol), CuI (10 mg, 0.06 mmol), 3,4,7,8-tetramethyl-1,10-phenanthroline (12 mg, 0.06 mmol), K₃PO₄ (38 mg, 0.18 mmol) in 1,4-dioxane (5 ml) was stirred for 16 h at 120° C. under nitrogen. The reaction mixture was concentrated and purified by silica gel column chromatography (MeOH: DCM=25:1) to afford the title compound as a yellow solid (5.0 mg).

### LCMS (ESI): 571.3 [M+1]⁺.

### EXAMPLE 50

### (S)-4-(1-(5-(4-fluorophenyl)-1-(4-(trifluoromethyl)benzyl)-1H-indole-7-carboxamido)ethyl) benzoic acid

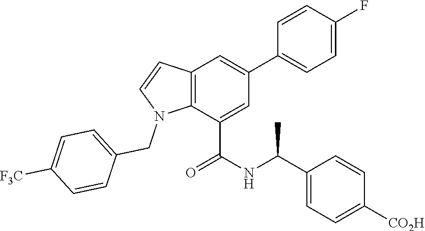 [SMILES: C[C@H](NC(=O)c1cc(-c2ccc(F)cc2)cc2ccn(Cc3ccc(C(F)(F)F)cc3)c12)c1ccc(C(=O)O)cc1]

Starting with methyl (S)-4-(1-aminoethyl)benzoate and the product of Step 2 of Example 1, the title product was obtained as a white solid by following the same procedures as described in Step 3, Step 4 and Step 5 of Example 1. LCMS (ESI): 561.3 [M+1]⁺.

### EXAMPLE 51

### (S)-4-(1-(5-phenyl-1-(4-(trifluoromethyl)benzyl)-1H-indole-7-carboxamido)ethyl) benzoic acid

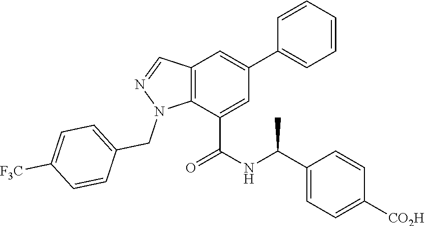 [SMILES: C[C@H](NC(=O)c1cc(-c2ccccc2)cc2cnn(Cc3ccc(C(F)(F)F)cc3)c12)c1ccc(C(=O)O)cc1]

Starting with methyl (S)-4-(1-(5-bromo-1-(4-(trifluoromethyl)benzyl)-1H-indole-7-carboxamido)ethyl)benzoate (30 mg) and phenylboronic acid, the title product was obtained as a white solid (12 mg) by following the same procedures as described for Example 1. LCMS (ES+): 543.4 [M+1]*.

### EXAMPLE 52

### (S)-4-(1-(5-(3-cyanophenyl)-1-(4-(trifluoromethyl)benzyl)-1H-indole-7-carboxamido)ethyl) benzoic acid

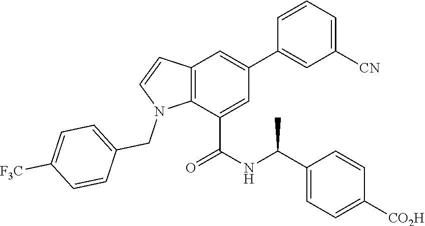 [SMILES: C[C@H](NC(=O)c1cc(-c2cccc(C#N)c2)cc2ccn(Cc3ccc(C(F)(F)F)cc3)c12)c1ccc(C(=O)O)cc1]

Starting with methyl (S)-4-(1-(5-bromo-1-(4-(trifluoromethyl)benzyl)-1H-indole-7-carboxamido)ethyl)benzoate (30 mg) and (3-cyanophenyl)boronic acid, the title product was obtained as a white solid (12 mg) by following the same procedures as described for Example 1. LCMS (ESI): 568.2 [M+1]⁺.

### EXAMPLE 53

### (S-4-(1-(5-(3,4-difluorophenyl)-1-(4-(trifluoromethyl)benzyl)-1H-indole-7-carboxamido)ethyl)benzoic acid

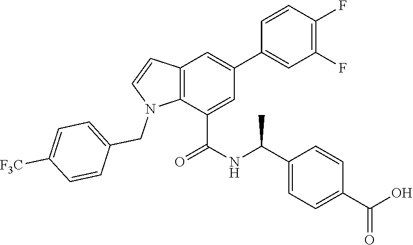 [SMILES: C[C@H](NC(=O)c1cc(-c2ccc(F)c(F)c2)cc2ccn(Cc3ccc(C(F)(F)F)cc3)c12)c1ccc(C(=O)O)cc1]

Starting with methyl (S)-4-(1-(5-bromo-1-(4-(trifluoromethyl)benzyl)-1H-indole-7-carboxamido)ethyl)benzoate (30 mg) and (3,4-difluorophenyl)boronic acid, the title product was obtained as a white solid (12 mg) by following the same procedures as described for Example 1. LCMS (ESI): 579.0 [M+1]⁺.

¹H NMR (500 MHz, DMSO-d₆) δ 12.82 (s, 1H), 8.88 (d, J=7.8 Hz, 1H), 8.04 (d, J=1.8 Hz, 1H), 7.87 (d, J=8.3 Hz, 2H), 7.87-7.79 (m, 1H), 7.65 (d, J=3.2 Hz, 1H), 7.63-7.52 (m, 2H), 7.53-7.45 (m, 3H), 7.44 (d, J=8.3 Hz, 2H), 6.95 (d, J=8.0 Hz, 2H), 6.72 (d, J=3.1 Hz, 1H), 5.66 (s, 2H), 5.06 (p, J=7.2 Hz, 1H), 1.26 (d, J=7.0 Hz, 3H).

### EXAMPLE 54

### (S)-4-(1-(5-(3,5-difluorophenyl)-1-(4-(trifluoromethyl)benzyl)-1H-indole-7-carboxamido)ethyl) benzoic acid

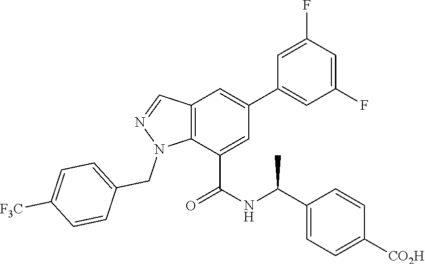 [SMILES: C[C@H](NC(=O)c1cc(-c2cc(F)cc(F)c2)cc2cnn(Cc3ccc(C(F)(F)F)cc3)c12)c1ccc(C(=O)O)cc1]

Starting with (3,5-difluorophenyl) boronic acid, the title product was obtained as a white solid by following the same procedure described in Example 50. LCMS (ESI): 579.4 [M+1]*.

### EXAMPLE 55

### (S)-4-(1-(1-(4-(trifluoromethyl)benzyl)-5-(3-(trifluoromethyl)phenyl)-1H-indole-7-carboxamido)ethyl) benzoic acid

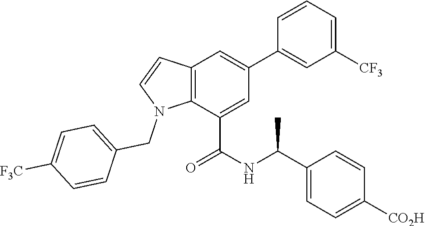 [SMILES: C[C@H](NC(=O)c1cc(-c2cccc(C(F)(F)F)c2)cc2ccn(Cc3ccc(C(F)(F)F)cc3)c12)c1ccc(C(=O)O)cc1]

Starting with (3-(trifluoromethyl)phenyl) boronic acid, the title product was obtained as a white solid by following the same procedures as described in Example 50. LCMS (ESI): 611.4 [M+1]*.

### EXAMPLE 56

### (S)-4-(1-(5-(3-fluoro-5-(trifluoromethyl)phenyl)-1-(4-(trifluoromethyl)benzyl)-1H-indole-7-carboxamido)ethyl)benzoic acid

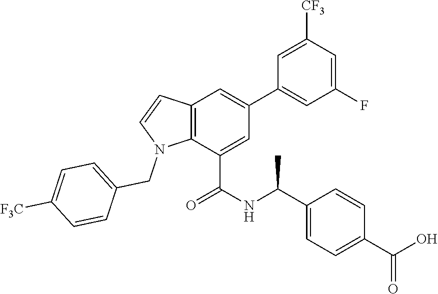 [SMILES: C[C@H](NC(=O)c1cc(-c2cc(F)cc(C(F)(F)F)c2)cc2ccn(Cc3ccc(C(F)(F)F)cc3)c12)c1ccc(C(=O)O)cc1]

Starting with (3-fluoro-5-(trifluoromethyl)phenyl)boronic acid, the title product was obtained as a white solid by following the same procedures as described in Example 50. LCMS (ES+): 629.2 [M+1]⁺.

### EXAMPLE 57

### 4-((4-(phenylamino)-1-(4-(trifluoromethyl)benzyl)-1H-indole-7-carboxamido)methyl)benzoic acid

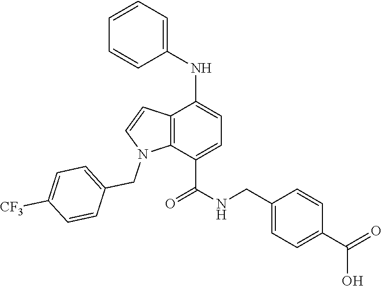 [SMILES: O=C(O)c1ccc(CNC(=O)c2ccc(Nc3ccccc3)c3ccn(Cc4ccc(C(F)(F)F)cc4)c23)cc1]

### Step 1 methyl 4-((4-(phenylamino)-1-(4-(trifluoromethyl)benzyl)-1H-indole-7-carboxamido)methyl)benzoate

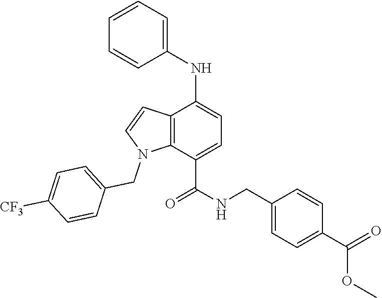 [SMILES: COC(=O)c1ccc(CNC(=O)c2ccc(Nc3ccccc3)c3ccn(Cc4ccc(C(F)(F)F)cc4)c23)cc1]

A mixture of the methyl 4-(1-(5-bromo-1-(4-(trifluoromethyl)benzyl)-1H-indole-7-carboxamido)cyclopropyl)benzoate (30 mg), aniline (30 mg), Xantphos (6.2 mg), Pd₂(dba)₃ (3.2 mg), Cs₂CO₃ (60 mg) in 1,4-dioxane (5 ml) was stirred for 3 h at 110° C. under nitrogen. The reaction mixture was then concentrated and the residue was purified by silica gel column chromatography (PE/EA=5:1) to afford the title compound as a yellow solid (22.0 mg). LCMS (ESI): 558.3 [M+1]⁺.

### Step 2 4-((4-(phenylamino)-1-(4-(trifluoromethyl)benzyl)-1H-indole-7-carboxamido)methyl)benzoic acid

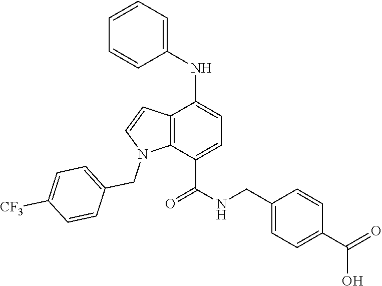 [SMILES: O=C(O)c1ccc(CNC(=O)c2ccc(Nc3ccccc3)c3ccn(Cc4ccc(C(F)(F)F)cc4)c23)cc1]

The title product was obtained as a white solid (12.0 mg) by following the same procedures as described in Step 5 of Example 1. LCMS (ESI): 544.2 [M+1]⁺.

¹H NMR (500 MHz, DMSO-d₆) δ 12.85 (s, 1H), 8.70 (t, J=6.1 Hz, 1H), 8.29 (s, 1H), 7.86-7.81 (m, 2H), 7.52 (d, J=8.2 Hz, 2H), 7.46 (d, J=3.3 Hz, 1H), 7.34-7.27 (m, 4H), 7.27-7.22 (m, 2H), 7.19 (d, J=8.0 Hz, 1H), 6.97 (d, J=8.0 Hz, 2H), 6.93 (tt, J=7.3, 1.3 Hz, 1H), 6.89 (d, J=3.2 Hz, 1H), 6.83 (d, J=8.1 Hz, 1H), 5.72 (s, 2H), 4.38 (d, J=6.0 Hz, 2H).

### EXAMPLE 58

### 4-((4-morpholino-1-(4-(trifluoromethyl)benzyl)-1H-indole-7-carboxamido)methyl)benzoic acid

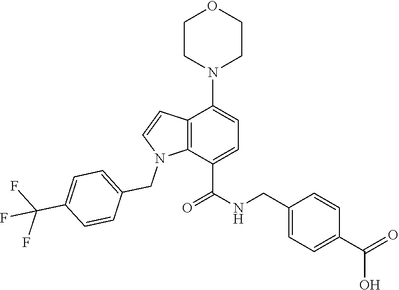 [SMILES: O=C(O)c1ccc(CNC(=O)c2ccc(N3CCOCC3)c3ccn(Cc4ccc(C(F)(F)F)cc4)c23)cc1]

Starting with methyl 4-((4-bromo-1-(4-(trifluoromethyl)benzyl)-1H-indole-7-carboxamido)methyl)benzoate (30 mg) and morpholine, the title product was obtained as a white solid (10 mg) by following the same procedures as described for Example 57. LCMS (ESI): 538.2 [M+1]⁺.

### EXAMPLE 59

### 4-((4-(4-methylpiperazin-1-yl)-1-(4-(trifluoromethyl)benzyl)-1H-indole-7-carboxamido)methyl)benzoic acid

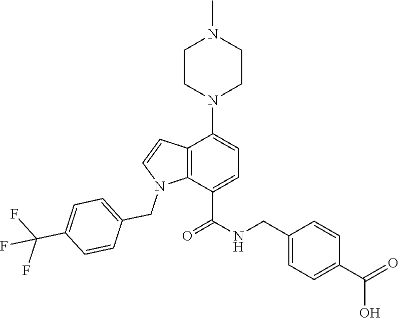 [SMILES: CN1CCN(c2ccc(C(=O)NCc3ccc(C(=O)O)cc3)c3c2ccn3Cc2ccc(C(F)(F)F)cc2)CC1]

Starting with methyl 4-((4-bromo-1-(4-(trifluoromethyl)benzyl)-1H-indole-7-carboxamido)methyl)benzoate (30 mg) and 1-methylpiperazine, the title product was obtained as a white solid (12 mg) by following the same procedures as described for Example 01. LCMS (ESI): 551.2 [M+1]⁺.

### EXAMPLE 60

### (R)-4-(1-(4-(phenylamino)-1-(4-(trifluoromethyl)benzyl)-1H-indole-7-carboxamido)ethyl)benzoic acid

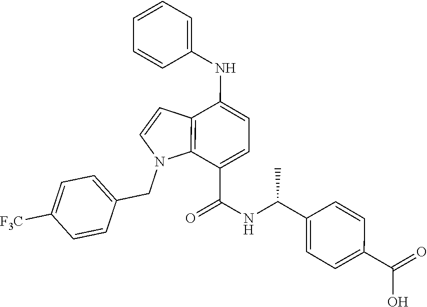 [SMILES: C[C@@H](NC(=O)c1ccc(Nc2ccccc2)c2ccn(Cc3ccc(C(F)(F)F)cc3)c12)c1ccc(C(=O)O)cc1]

Starting with methyl (R)-4-(1-(4-bromo-1-(4-(trifluoromethyl)benzyl)-1H-indole-7-carboxamido)ethyl)benzoate (30 mg) and aniline, the title product was obtained as a white solid (14 mg) by following the same procedures as described for Example 01. LCMS (ESI): 558.3 [M+1]⁺.

### EXAMPLE 61

### 4-(1-(4-((3-cyanophenyl)amino)-1-(4-(trifluoromethyl)benzyl)-1H-indole-7-carboxamido)cyclopropyl)benzoic acid

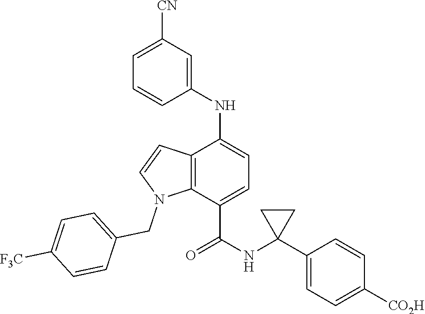 [SMILES: N#Cc1cccc(Nc2ccc(C(=O)NC3(c4ccc(C(=O)O)cc4)CC3)c3c2ccn3Cc2ccc(C(F)(F)F)cc2)c1]

Starting with 3-aminobenzonitrile (40 mg), the title product was obtained as a white solid (14 mg) by following the same procedure described for Example 44. LCMS (ES+): 595.5[M+1]⁺.

### EXAMPLE 62

### 4-(1-(4-((4-cyanophenyl)amino)-1-(4-(trifluoromethyl)benzyl)-1H-indole-7-carboxamido)cyclopropyl)benzoic acid

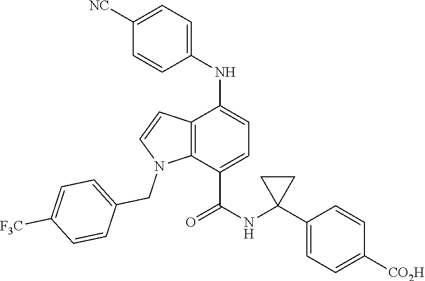 [SMILES: N#Cc1ccc(Nc2ccc(C(=O)NC3(c4ccc(C(=O)O)cc4)CC3)c3c2ccn3Cc2ccc(C(F)(F)F)cc2)cc1]

Starting with 4-aminobenzonitrile (40 mg), the title product was obtained as a white solid (16 mg) by following the same procedure described for Example 44. LCMS (ES+): 595.5[M+1]⁺.

### EXAMPLE 63

### 4-(1-(4-((3,4-difluorophenyl)amino)-1-(4-(trifluoromethyl)benzyl)-1H-indole-7-carboxamido)cyclopropyl)benzoic acid

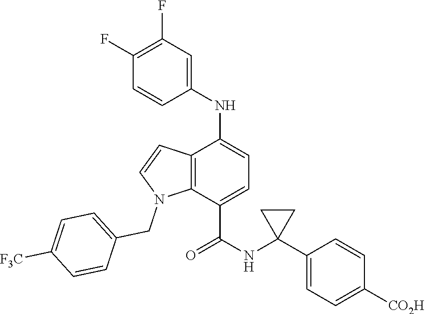 [SMILES: O=C(O)c1ccc(C2(NC(=O)c3ccc(Nc4ccc(F)c(F)c4)c4ccn(Cc5ccc(C(F)(F)F)cc5)c34)CC2)cc1]

Starting with 3,4-difluoroaniline (40 mg), the title product was obtained as a white solid (17 mg) by following the same procedure described for Example 44.

### LCMS (ES+): 606.4[M+1]⁺.

### EXAMPLE 64

### 4-(1-(4-((4-fluorophenyl)amino)-1-(4-(trifluoromethyl)benzyl)-1H-indole-7-carboxamido) cyclopropyl)benzoic acid

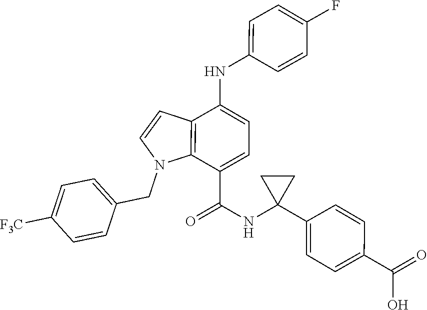 [SMILES: O=C(O)c1ccc(C2(NC(=O)c3ccc(Nc4ccc(F)cc4)c4ccn(Cc5ccc(C(F)(F)F)cc5)c34)CC2)cc1]

### Step 1 methyl 4-bromo-1-(4-(trifluoromethyl)benzyl)-1H-indole-7-carboxylate

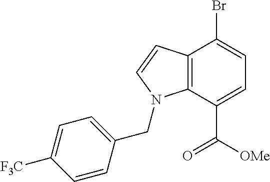 [SMILES: COC(=O)c1ccc(Br)c2ccn(Cc3ccc(C(F)(F)F)cc3)c12]

To a mixture of methyl 4-bromo-1H-indole-7-carboxylate (500 mg) and Cs₂CO₃ (769 mg) in anhydrous DMF (10 mL) at 0° C. was added 1-(bromomethyl)-4-(trifluoromethyl)benzene (564 mg, 2.36 mmol). The reaction mixture was then warmed to rt and stirred overnight. The reaction mixture was poured into 100 mL ice-water, extracted 3×50 mL EtOAc. The combined organic layer was washed with 50 mL saturated brine, dried over anhydrous Na₂SO₄ and concentrated. The residue was purified by silica gel column chromatography (petroleum ether/ethyl acetate: from 100:0 to 10:1) to afford the title compound as a white solid (500 mg). LCMS (ESI): 412.2 [M+1]⁺.

### Step 2 4-bromo-1-(4-(trifluoromethyl)benzyl)-1H-indole-7-carboxylic acid

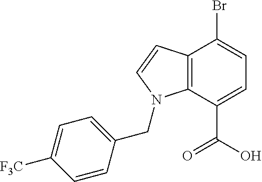 [SMILES: O=C(O)c1ccc(Br)c2ccn(Cc3ccc(C(F)(F)F)cc3)c12]

To a solution of the product of Step 1 (500 mg) in MeOH/H₂O (10 mL/2.0 mL) was added LiOH—H₂O (254 mg) at r.t. The reaction mixture was stirred at 60° C. for 1 h. After being cooled to 0° C., the reaction mixture was treated with 6 N HCl to adjusted pH≈6. The reaction mixture was extracted 3×30 mL EtOAc. The combined organic layer washed with 20 mL saturated brine, dried over anhydrous Na₂SO₄, concentrated. The residue was purified by silica gel column chromatography (DCM/MeOH: from 100:0 to 10:1) to afford the title compound as a white solid (479 mg). LCMS (ESI): 398.2 [M+1]⁺.

### Step 3 methyl 4-(1-(4-bromo-1-(4-(trifluoromethyl)benzyl)-1H-indole-7-carboxamido)cyclopropyl)benzoate

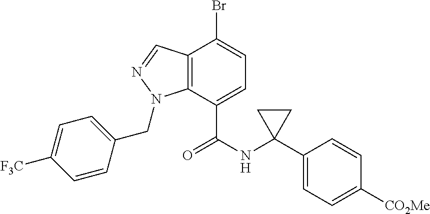 [SMILES: COC(=O)c1ccc(C2(NC(=O)c3ccc(Br)c4cnn(Cc5ccc(C(F)(F)F)cc5)c34)CC2)cc1]

To a mixture of the product of Step 2 (398 mg), methyl 4-(1-aminocyclopropyl)benzoate (215 mg) and HATU (456 mg) in anhydrous DMF (100 mL) at 0° C. was added DIPEA (0.52 mL, 3.0 mmol). After stirring at rt overnight, the reaction mixture was quenched with 100 mL water and extracted 3×100 mL EtOAc. The combined organic layer was washed with 100 mL saturated brine, dried over anhydrous Na₂SO₄ and concentrated. The residue was purified by silica gel column chromatography (petroleum ether/ethyl acetate: from 100:0 to 5:1) to afford the title compound as a white solid (0.5 g). LCMS (ESI): 571.1 [M+1]⁺.

### Step 4 methyl 4-(1-(4-((4-fluorophenyl)amino)-1-(4-(trifluoromethyl)benzyl)-1H-indole-7-carboxamido)cyclopropyl)benzoate

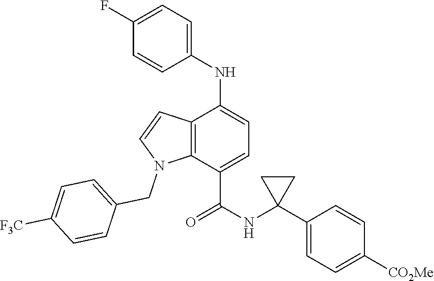 [SMILES: COC(=O)c1ccc(C2(NC(=O)c3ccc(Nc4ccc(F)cc4)c4ccn(Cc5ccc(C(F)(F)F)cc5)c34)CC2)cc1]

A mixture of the methyl 4-(1-(4-bromo-1-(4-(trifluoromethyl)benzyl)-1H-indole-7-carboxamido)cyclopropyl)benzoate (30 mg, 0.06 mmol), 4-Fluoroaniline (30 mg, 0.36 mmol), Xantphos (6.2 mg, 0.012 mmol), Pd₂(dba)₃ (3.2 mg, 0.006 mmol), Cs₂CO₃ (60 mg, 0.18 mmol) in 1,4-dioxane (5 ml) was stirred for 16 h at 120° C. under nitrogen. The reaction mixture was concentrated and purified by silica gel column chromatography (PE/EA=5:1) to afford the title compound as a yellow solid (2.0 mg). LCMS (ESI):602.3 [M+1]⁺.

### Step 5 4-(1-(4-((4-fluorophenyl)amino)-1-(4-(trifluoromethyl)benzyl)-1H-indole-7-carboxamido)cyclopropyl)benzoic acid

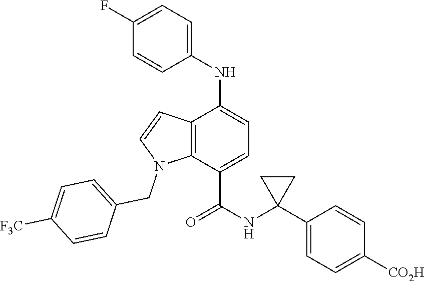 [SMILES: O=C(O)c1ccc(C2(NC(=O)c3ccc(Nc4ccc(F)cc4)c4ccn(Cc5ccc(C(F)(F)F)cc5)c34)CC2)cc1]

To a solution of the product of Step 4 (42 mg) in MeOH/H₂O (5 mL/0.5 mL) was added LiOH—H₂O (16 mg). The reaction mixture was stirred at 60° C. for 1 h. After being cooled to 0° C., the mixture was treated with 6 N HCl to pH≈6. The reaction mixture was then extracted 3×20 mL EtOAc. The combined organic layer was washed with 20 mL brine, dried over anhydrous Na₂SO₄ and concentrated. The residue was purified by silica gel column chromatography (DCM/MeOH=10:1) to afford the title compound as a white solid (12.1 mg). LCMS (ESI): 588.4 [M+1]⁺.

¹HNMR (400 MHz, DMSO-d₆) δ 8.91 (s, 1H), 8.27 (s, 1H), 7.75 (d, J=8.3 Hz, 2H), 7.59 (d, J=8.1 Hz, 2H), 7.37 (d, J=3.2 Hz, 1H), 7.31-7.22 (m, 3H), 7.20-7.13 (m, 4H), 6.94 (d, J=8.1 Hz, 2H), 6.88 (d, J=3.3 Hz, 1H), 6.76 (d, J=8.0 Hz, 1H), 5.74 (s, 2H), 1.21-1.16 (m, 2H), 0.99-0.95 (m, 2H).

### EXAMPLE 65

### 4-(1-(4-((3-chloro-4-fluorophenyl)amino)-1-(4-(trifluoromethyl)benzyl)-1H-indole-7-carboxamido)cyclopropyl)benzoic acid

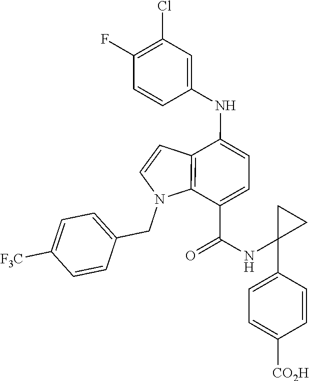 [SMILES: O=C(O)c1ccc(C2(NC(=O)c3ccc(Nc4ccc(F)c(Cl)c4)c4ccn(Cc5ccc(C(F)(F)F)cc5)c34)CC2)cc1]

Starting with 3-chloro-4-fluoroaniline (70 mg), the title product was obtained as a white solid (25 mg) by following the same procedure described in Example 64. LCMS (ESI): 622.1 [M+1]⁺.

¹HNMR (400 MHz, DMSO-d₆) δ 8.97 (s, 1H), 8.45 (s, 1H), 7.76 (d, J=8.1 Hz, 2H), 7.59 (d, J=8.1 Hz, 2H), 7.40 (d, J=3.2 Hz, 1H), 7.36-7.30 (m, 3H), 7.24-7.16 (m, 3H), 6.97-6.92 (m, 2H), 6.88 (d, J=7.9 Hz, 1H), 6.83 (d, J=3.2 Hz, 1H), 5.74 (s, 2H), 1.21-1.15 (m, 2H), 0.99-0.93 (m, 2H).

### EXAMPLE 66

### 4-(1-(4-((3,5-difluorophenyl)amino)-1-(4-(trifluoromethyl)benzyl)-1H-indole-7-carboxamido)cyclopropyl)benzoic acid

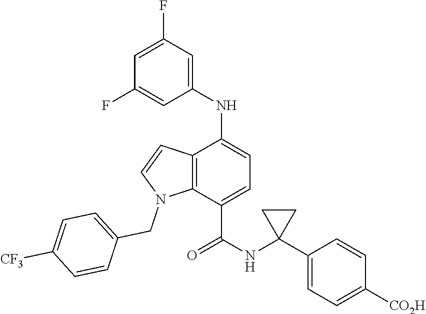 [SMILES: O=C(O)c1ccc(C2(NC(=O)c3ccc(Nc4cc(F)cc(F)c4)c4ccn(Cc5ccc(C(F)(F)F)cc5)c34)CC2)cc1]

Starting with 3,5-difluoroaniline (40 mg), the title product was obtained as a white solid (25 mg) by following the same procedure described for Example 64.

LCMS (ESI): 606.3[M+1]⁺. ¹H NMR (400 MHz, DMSO-d₆) δ 9.05 (s, 1H), 8.79 (s, 1H), 7.77 (d, J=8.2 Hz, 2H), 7.60 (s, 1H), 7.44 (d, J=3.3 Hz, 1H), 7.37 (s, 1H), 7.20 (d, J=8.3 Hz, 2H), 7.04 (d, J=7.9 Hz, 1H), 6.94 (s, 1H), 6.79-6.75 (m, 3H), 6.61-6.57 (m, 1H), 5.74 (s, 2H), 1.19-1.16 (m, 2H), 0.96-0.94 (m, 2H).

### EXAMPLE 67

### 4-(1-(4-((3-fluorophenyl)amino)-1-(4-(trifluoromethyl)benzyl)-1H-indole-7-carboxamido)cyclopropyl)benzoic acid

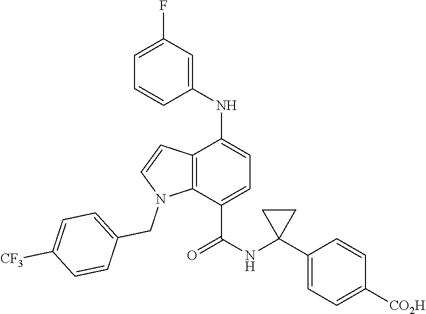 [SMILES: O=C(O)c1ccc(C2(NC(=O)c3ccc(Nc4cccc(F)c4)c4ccn(Cc5ccc(C(F)(F)F)cc5)c34)CC2)cc1]

Starting with 3-fluoroaniline (40 mg), the title product was obtained as a white solid (25 mg) by following the same procedure described for Example 64.

LCMS (ESI): 588.47[M+1]⁺. ¹H NMR (400 MHz, DMSO-d₆) δ 9.00 (s, 1H), 8.54 (s, 1H), 7.76 (d, J=8.1 Hz, 2H), 7.59 (d, J=8.1 Hz, 2H), 7.41 (d, J=3.4 Hz, 1H), 7.34 (d, J=8.0 Hz, 1H), 7.32-7.27 (m, 1H), 7.19 (d, J=8.1 Hz, 2H), 7.08-7.05 (m, 1H), 7.01-6.92 (m, 4H), 6.84 (d, J=3.2 Hz, 1H), 6.70-6.66 (m, 1H), 5.75 (d, J=9.2 Hz, 2H), 1.20-1.14 (m, 2H), 0.98-0.94 (m, 2H).

### EXAMPLE 68

### 4-((4-((3-cyanophenyl)amino)-1-(4-(trifluoromethyl)benzyl)-1H-indole-7-carboxamido)methyl)benzoic acid

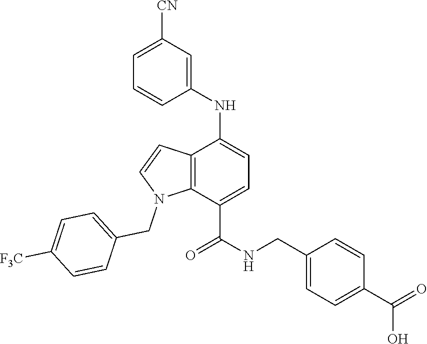 [SMILES: N#Cc1cccc(Nc2ccc(C(=O)NCc3ccc(C(=O)O)cc3)c3c2ccn3Cc2ccc(C(F)(F)F)cc2)c1]

Starting with 3-aminobenzonitrile and methyl 4-(aminomethyl)benzoate hydrochloride, the title product was obtained as a white solid by following the same procedure described in Example 64. LCMS (ESI): 569.2 [M+1]⁺.

¹HNMR (400 MHz, DMSO-d₆) δ 8.76 (t, J=5.9 Hz, 1H), 8.65 (s, 1H), 7.83 (d, J=8.5 Hz, 2H), 7.55-7.42 (m, 6H), 7.31-7.21 (m, 4H), 7.01-6.97 (m, 3H), 6.78 (d, J=3.2 Hz, 1H), 5.71 (s, 2H), 4.41-4.35 (m, 2H).

### EXAMPLE 69

### 4-((4-((3,5-difluorophenyl)amino)-1-(4-(trifluoromethyl)benzyl)-1H-indole-7-carboxamido)methyl)benzoic acid

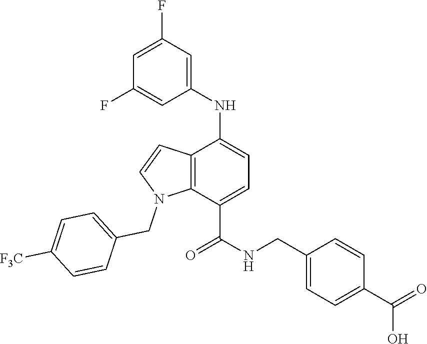 [SMILES: O=C(O)c1ccc(CNC(=O)c2ccc(Nc3cc(F)cc(F)c3)c3ccn(Cc4ccc(C(F)(F)F)cc4)c23)cc1]

Starting with 3,5-difluoroaniline and methyl 4-(aminomethyl)benzoate hydrochloride, the title product was obtained as a white solid by following the same procedure described in Example 64. LCMS (ESI): 580.2 [M+1]⁺.

¹HNMR (400 MHz, DMSO-d₆) δ 8.78 (t, J=6.0 Hz, 1H), 8.74 (s, 1H), 7.83 (d, J=8.0 Hz, 2H), 7.55-7.50 (m, 3H), 7.30 (d, J=8.0 Hz, 2H), 7.25 (d, J=7.9 Hz, 1H), 7.01-6.97 (m, 3H), 6.77-7.74 (m, 3H), 6.61-6.58 (m, 1H), 5.70 (s, 2H), 4.42-4.36 (m, 2H).

### EXAMPLE 70

### 4-(1-(4-((3-chloro-4-fluorophenyl)amino)-1-(4-(trifluoromethyl)benzyl)-1H-indole-7-carboxamido)cyclopropyl)benzoic acid

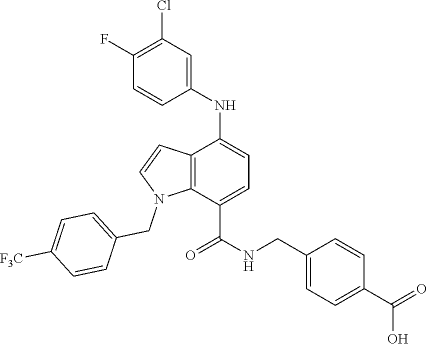 [SMILES: O=C(O)c1ccc(CNC(=O)c2ccc(Nc3ccc(F)c(Cl)c3)c3ccn(Cc4ccc(C(F)(F)F)cc4)c23)cc1]

Starting with 3-chloro-4-fluoroaniline and methyl 4-(aminomethyl)benzoate hydrochloride, the title product was obtained as a white solid (25 mg) by following the same procedure described in Example 64.

### LCMS (ESI): 596.2 [M+1]⁺.

¹HNMR (400 MHz, DMSO-d₆) δ 8.02-7.97 (m, 2H), 7.47 (d, J=8.0 Hz, 2H), 7.24-7.21 (m, 3H), 7.19-7.15 (m, 2H), 7.11-7.06 (m, 1H), 7.06-7.00 (m, 1H), 6.97 (d, J=8.0 Hz, 2H), 6.76 (d, J=8.0 Hz, 1H), 6.57 (d, J=3.2 Hz, 1H), 5.89 (t, J=5.8 Hz, 1H), 5.72 (s, 2H), 4.43-4.38 (m, 2H).

### EXAMPLE 71

### ((4-((4-chloro-3-fluorophenyl)amino)-1-(4-(trifluoromethyl)benzyl)-1H-indole-7-carboxamido)methyl)benzoic acid acid

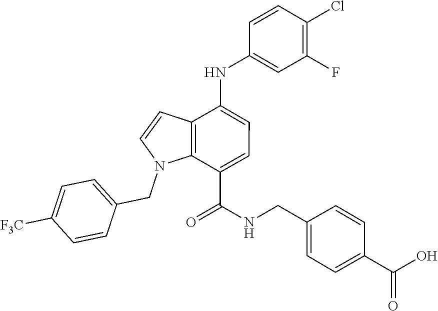 [SMILES: O=C(O)c1ccc(CNC(=O)c2ccc(Nc3ccc(Cl)c(F)c3)c3ccn(Cc4ccc(C(F)(F)F)cc4)c23)cc1]

Starting with 4-chloro-3-fluoroaniline and methyl 4-(aminomethyl)benzoate hydrochloride the title product was obtained as a white solid by following the same procedures described in Example 64. LCMS (ESI): 596.4 [M+1]⁺.

¹H NMR (400 MHz, DMSO-d₆) δ 8.69 (t, J=6.0 Hz, 1H), 8.63 (s, 1H), 7.80 (d, J=7.6 Hz, 2H), 7.56 (d, J=8.1 Hz, 2H), 7.51 (d, J=3.3 Hz, 1H), 7.41 (t, J=8.7 Hz, 1H), 7.24-7.15 (m, 3H), 7.04-6.98 (m, 1H), 7.01 (dd, J=8.7, 2.8 Hz, 3H), 6.92 (d, J=7.9 Hz, 1H), 6.77 (d, J=3.2 Hz, 1H), 5.70 (s, 2H), 4.33 (d, J=5.9 Hz, 2H).

### EXAMPLE 72

### 4-((4-((3-fluorophenyl)amino)-1-(4-(trifluoromethyl)benzyl)-1H-indole-7-carboxamido)methyl)benzoic acid

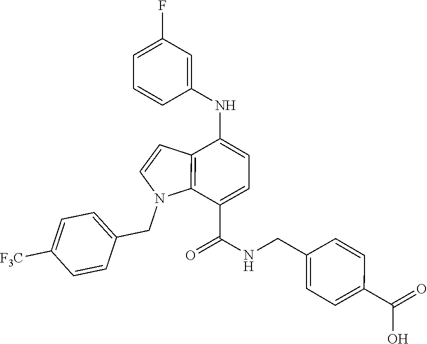 [SMILES: O=C(O)c1ccc(CNC(=O)c2ccc(Nc3cccc(F)c3)c3ccn(Cc4ccc(C(F)(F)F)cc4)c23)cc1]

Starting with 3-fluoroaniline and methyl 4-(aminomethyl)benzoate hydrochloride, the title product was obtained as a white solid by following the same procedure described in Example 64. LCMS (ESI):562.2 [M+1]⁺.

¹HNMR (400 MHz, DMSO-d₆) δ 8.74 (t, J=6.0 Hz, 1H), 8.49 (s, 1H), 7.82 (d, J=8.1 Hz, 2H), 7.52 (d, J=8.1 Hz, 2H), 7.48 (d, J=3.2 Hz, 1H), 7.28 (dd, J=7.9, 4.7 Hz, 3H), 7.22 (d, J=8.0 Hz, 1H), 7.02 (dd, J=8.1, 2.0 Hz, 1H), 6.99-6.90 (m, 4H), 6.81 (d, J=3.3 Hz, 1H), 6.66 (td, J=8.5, 2.5 Hz, 1H), 5.70 (s, 2H), 4.37 (d, J=6.0 Hz, 2H).

### EXAMPLE 73

### ((4-((4-chlorophenyl)amino)-1-(4-(trifluoromethyl)benzyl)-1H-indole-7-carboxamido)methyl)benzoic acid

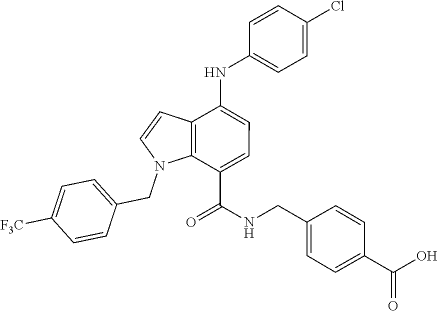 [SMILES: O=C(O)c1ccc(CNC(=O)c2ccc(Nc3ccc(Cl)cc3)c3ccn(Cc4ccc(C(F)(F)F)cc4)c23)cc1]

Starting with 4-chloro-aniline and methyl 4-(aminomethyl)benzoate hydrochloride the title product was obtained as a white solid by following the same procedures described in Example 64. LCMS (ESI): 578.3 [M+1]⁺.

¹HNMR (400 MHz, DMSO-d₆) δ 8.69 (t, J=6.1 Hz, 1H), 8.40 (s, 1H), 7.83 (d, J=7.8 Hz, 2H), 7.53 (d, J=8.1 Hz, 2H), 7.47 (d, J=3.3 Hz, 1H), 7.32 (d, J=8.7 Hz, 2H), 7.26 (d, J=7.7 Hz, 2H), 7.20 (d, J=3.6 Hz, 2H), 6.99 (d, J=8.0 Hz, 2H), 6.86-6.80 (m, 2H), 5.71 (s, 2H), 4.37 (d, J=6.0 Hz, 2H).

### EXAMPLE 74

### (S)-4-(1-(4-(phenylamino)-1-(4-(trifluoromethyl)benzyl)-1H-indole-7-carboxamido)ethyl)benzoic acid

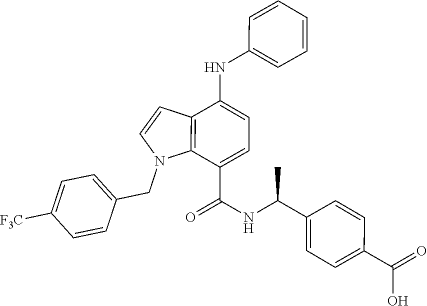 [SMILES: C[C@H](NC(=O)c1ccc(Nc2ccccc2)c2ccn(Cc3ccc(C(F)(F)F)cc3)c12)c1ccc(C(=O)O)cc1]

Starting with aniline and (S)-methyl 4-(1-aminoethyl)benzoate, the title product was obtained as a white solid by following the same procedure described in Example 64. LCMS (ESI): 558.2 [M+1]⁺.

¹HNMR (400 MHz, DMSO-d₆) δ 12.66 (s, 1H), 8.60 (d, J=8.0 Hz, 1H), 8.26 (s, 1H), 7.88-7.81 (m, 2 H), 7.48-7.44 (m, 3H), 7.41 (d, J=8.2 Hz, 2H), 7.29 (dd, J=8.5, 7.2 Hz, 2H), 7.24-7.21 (m, 2H), 7.14 (d, J=8.0 Hz, 1H), 6.95-6.88 (m, 3H), 6.88-6.80 (m, 2H), 5.65 (s, 2H), 5.07-5.01 (m, 1H), 1.24-1.22 (m, 3H).

### EXAMPLE 75

### 4-((5-(3-chlorophenyl)-1-(4-(trifluoromethyl)benzyl)-1H-indole-7-carboxamido)methyl)benzoic acid

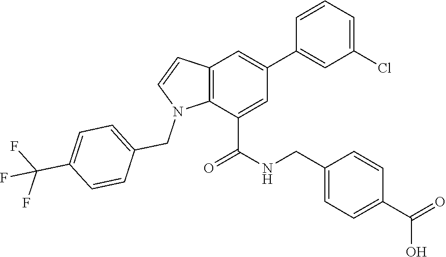 [SMILES: O=C(O)c1ccc(CNC(=O)c2cc(-c3cccc(Cl)c3)cc3ccn(Cc4ccc(C(F)(F)F)cc4)c23)cc1]

### Step 1 methyl 5-bromo-1-(4-(trifluoromethyl)benzyl)-1H-indole-7-carboxylate

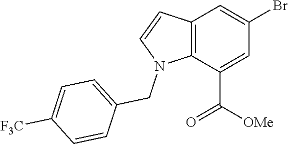 [SMILES: COC(=O)c1cc(Br)cc2ccn(Cc3ccc(C(F)(F)F)cc3)c12]

To a mixture of methyl 5-bromo-1H-indole-7-carboxylate (500 mg) and Cs₂CO₃ (769 mg) in anhydrous DMF (10 mL) at 0° C. was added 1-(bromomethyl)-4-(trifluoromethyl)benzene (564 mg). The reaction mixture was then warmed to rt and stirred overnight. The reaction mixture was poured into 100 mL ice-water, extracted 3×50 mL EtOAc. The combined organic layer was washed with 50 mL brine, dried over anhydrous Na₂SO₄ and concentrated. The residue was purified by silica gel column chromatography (petroleum ether/ethyl acetate: from 100:0 to 10:1) to afford the title compound as a white solid (500 mg).

### LCMS (ESI): 412.2 [M+1]⁺.

### Step 2 5-bromo-1-(4-(trifluoromethyl)benzyl)-1H-indole-7-carboxylic acid

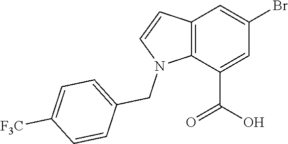 [SMILES: O=C(O)c1cc(Br)cc2ccn(Cc3ccc(C(F)(F)F)cc3)c12]

To a solution of the product of Step 1 (500 mg) in MeOH/H₂O (10 mL/2 mL) was added LiOH—H₂O (254 mg) at rt. The reaction mixture was stirred at 60° C. for 1 h. After being cooled to 0° C., the reaction mixture was treated with 6 N HCl to pH≈6. The reaction mixture was extracted 3×30 mL EtOAc. The combined organic layer washed with 20 mL brine, dried over anhydrous Na₂SO₄, concentrated. The residue was purified by silica gel column chromatography (DCM/MeOH: from 100:0 to 10:1) to afford the title compound as a white solid (479 mg). LCMS (ESI): 398.2 [M+1]⁺.

### Step 3 methyl 4-((5-bromo-1-(4-(trifluoromethyl)benzyl)-1H-indole-7-carboxamido)methyl)benzoate

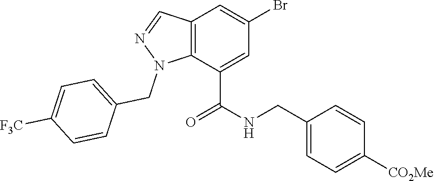 [SMILES: COC(=O)c1ccc(CNC(=O)c2cc(Br)cc3cnn(Cc4ccc(C(F)(F)F)cc4)c23)cc1]

To a mixture of the product of Step 2 (398 mg), methyl 4-(aminomethyl)benzoate (215 mg) and HATU (456 mg) in anhydrous DMF (10 mL) at 0° C. was added DIPEA (0.52 mL). After stirring at rt overnight, the reaction mixture was quenched with 10 mL water and extracted 3×100 mL EtOAc. The combined organic layer was washed with 10 mL brine, dried over anhydrous Na₂SO₄ and concentrated. The residue was purified by silica gel column chromatography (petroleum ether/ethyl acetate: from 100:0 to 5:1) to afford the title compound as a white solid (0.5 g). LCMS (ESI): 545.1 [M+1]⁺.

### Step 4 methyl 4-((5-(3-chlorophenyl)-1-(4-(trifluoromethyl)benzyl)-1H-indole-7-carboxamido)methyl)benzoate

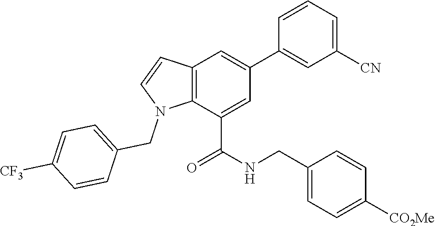 [SMILES: COC(=O)c1ccc(CNC(=O)c2cc(-c3cccc(C#N)c3)cc3ccn(Cc4ccc(C(F)(F)F)cc4)c23)cc1]

A mixture of the product of Step 3 (30 mg), (3-chlorophenyl)boronic acid (38 mg), Pd(dppf)₂Cl₂ (4 mg), K₂CO₃ (38 mg) in EtOH/H₂O (5 ml/1 mL) was stirred for 2 h at 70° C. under nitrogen. The reaction mixture was concentrated and purified by silica gel column chromatography (PE/EA=5:1) to afford the title compound as a yellow solid (27 mg). LCMS (ESI): 577.2 [M+1]⁺.

### Step 5 4-((5-(3-chlorophenyl)-1-(4-(trifluoromethyl)benzyl)-1H-indole-7-carboxamido)methyl)benzoic acid

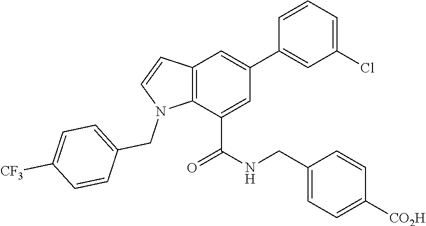 [SMILES: O=C(O)c1ccc(CNC(=O)c2cc(-c3cccc(Cl)c3)cc3ccn(Cc4ccc(C(F)(F)F)cc4)c23)cc1]

To a solution of the product of Step 4 (42 mg) in MeOH/H₂O (5 mL/0.5 mL) was added LiOH—H₂O (16 mg). The reaction mixture was stirred at 60° C. for 1 h. After being cooled to 0° C., the mixture was treated with 6 N HCl to pH≈6. The reaction mixture was then extracted 3×20 mL EtOAc. The combined organic layer was washed with 20 mL saturated brine, dried over anhydrous Na₂SO₄ and concentrated. The residue was purified by silica gel column chromatography (DCM/MeOH=10:1) to afford the title compound as a white solid (10.1 mg). LCMS (ESI): 577.2 [M+1]⁺.

¹HNMR (400 MHz, DMSO-d₆) δ 12.88 (s, 1H), 9.01 (t, J=5.9 Hz, 1H), 8.08 (d, J=1.7 Hz, 1H), 7.87-7.77 (m, 3H), 7.72 (d, J=7.7 Hz, 1H), 7.66 (d, J=3.1 Hz, 1H), 7.58-7.44 (m, 4H), 7.40 (dd, J=7.9, 2.1 Hz, 1H), 7.29 (d, J=8.1 Hz, 2H), 6.98 (d, J=8.0 Hz, 2H), 6.75 (d, J=3.1 Hz, 1H), 5.74 (s, 2H), 4.44 (d, J=5.8 Hz, 2H).

### EXAMPLE 76

### 4-((5-(3-methoxyphenyl)-1-(4-(trifluoromethyl)benzyl)-1H-indole-7-carboxamido)methyl)benzoic acid

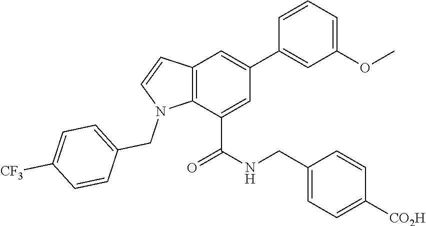 [SMILES: COc1cccc(-c2cc(C(=O)NCc3ccc(C(=O)O)cc3)c3c(ccn3Cc3ccc(C(F)(F)F)cc3)c2)c1]

Starting with 3-methoxyphenylboronic acid, the title product was obtained as a white solid (9 mg) by following the same procedures as described in Example 75. LCMS (ESI): 559.2 [M+1]⁺.

¹HNMR (400 MHz, DMSO-d₆) δ 12.85 (s, 1H), 9.02 (t, J=5.9 Hz, 1H), 8.03 (d, J=1.7 Hz, 1H), 7.87-7.82 (m, 2H), 7.64 (d, J=3.2 Hz, 1H), 7.56-7.51 (m, 3H), 7.38 (t, J=7.9 Hz, 1H), 7.33-7.25 (m, 4H), 6.98 (d, J=8.1 Hz, 2H), 6.91 (dd, J=8.2, 2.5 Hz, 1H), 6.74 (d, J=3.1 Hz, 1H), 5.73 (s, 2H), 4.43 (d, J=5.9 Hz, 2H), 3.83 (s, 3H).

### EXAMPLE 77

### ((5-(3-cyano-5-fluorophenyl)-1-(4-(trifluoromethyl)benzyl)-1H-indole-7-carboxamido)methyl)benzoic acid

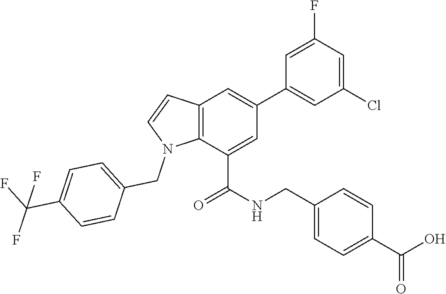 [SMILES: O=C(O)c1ccc(CNC(=O)c2cc(-c3cc(F)cc(Cl)c3)cc3ccn(Cc4ccc(C(F)(F)F)cc4)c23)cc1]

Starting with (3-cyano-5-fluorophenyl)boronic acid (98 mg), the title product was obtained as a white solid (20 mg) by following the same procedure described for Example 75. LCMS (ESI): 572.5[M+1]⁺.

¹HNMR (400 MHz, DMSO-d₆) δ 8.97 (t, J=5.9 Hz, 1H), 8.21 (d, J=1.8 Hz, 1H), 8.15 (d, J=1.7 Hz, 1H), 8.05-8.01 (m, 1H), 7.85 (d, J=8.2 Hz, 2H), 7.83-7.78 (m, 1H), 7.69 (d, J=3.2 Hz, 1H), 7.67 (d, J=1.9 Hz, 1H), 7.55 (d, J=8.1 Hz, 2H), 7.30 (d, J=8.1 Hz, 2H), 6.99 (d, J=8.0 Hz, 2H), 6.77 (d, J=3.2 Hz, 1H), 5.76 (s, 2H), 4.46 (d, J=5.8 Hz, 2H).

### EXAMPLE 78

### 4-((5-(pyridin-3-yl)-1-(4-(trifluoromethyl)benzyl)-1H-indole-7-carboxamido)methyl)benzoic acid

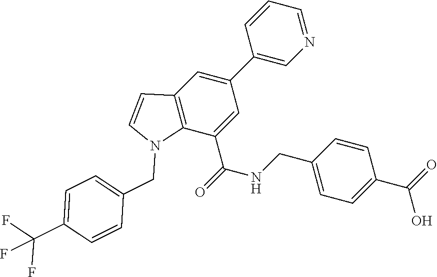 [SMILES: O=C(O)c1ccc(CNC(=O)c2cc(-c3cccnc3)cc3ccn(Cc4ccc(C(F)(F)F)cc4)c23)cc1]

Starting with pyridin-3-ylboronic acid, the title product was obtained as a white solid (10 mg) by following the same procedures as described in Example 75. LCMS (ESI): 530.2 [M+1]⁺.

¹HNMR (400 MHz, DMSO-d₆) δ 12.89 (s, 1H), 9.00 (t, J=5.9 Hz, 1H), 8.97 (d, J=2.3 Hz, 1H), 8.55 (dd, J=4.7, 1.6 Hz, 1H), 8.19-8.08 (m, 2H), 7.84 (d, J=8.1 Hz, 2H), 7.67 (d, J=3.2 Hz, 1H), 7.58 (d, J=1.8 Hz, 1H), 7.54 (d, J=8.1 Hz, 2H), 7.49 (dd, J=8.0, 4.7 Hz, 1H), 7.30 (d, J=8.2 Hz, 2H), 6.99 (d, J=8.0 Hz, 2H), 6.76 (d, J=3.2 Hz, 1H), 5.74 (s, 2H), 4.43 (d, J=5.8 Hz, 2H).

### EXAMPLE 79

### 4-((5-(3-cyano-4-fluorophenyl)-1-(4-(trifluoromethyl)benzyl)-1H-indole-7-carboxamido)methyl)benzoic

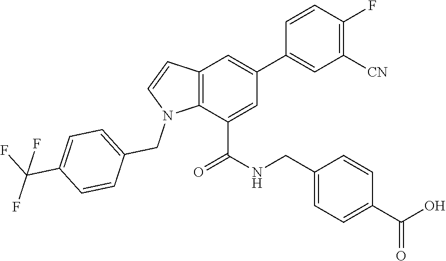 [SMILES: N#Cc1cc(-c2cc(C(=O)NCc3ccc(C(=O)O)cc3)c3c(ccn3Cc3ccc(C(F)(F)F)cc3)c2)ccc1F]

Starting with (3-cyano-4-fluorophenyl)boronic acid (98 mg), the title product was obtained as a white solid (20 mg) by following the same procedure described for Example 75. LCMS (ESI): 572.5[M+1]⁺.

¹HNMR (400 MHz, DMSO-d₆) δ 8.96 (t, J=5.9 Hz, 1H), 8.30-8.28 (m, 1H), 8.16-8.13 (m, 1H), 8.11 (d, J=1.8 Hz, 1H), 7.84 (d, J=8.0 Hz, 2H), 7.66 (d, J=3.2 Hz, 1H), 7.63 (t, J=9.0 Hz, 1H), 7.59 (d, J=1.8 Hz, 1H), 7.54 (d, J=8.1 Hz, 2H), 7.29 (d, J=8.0 Hz, 2H), 6.98 (d, J=8.1 Hz, 2H), 6.75 (d, J=3.2 Hz, 1H), 5.74 (s, 2H), 4.43 (d, J=5.9 Hz, 2H).

### EXAMPLE 80

### 4-((5-(pyridin-4-yl)-1-(4-(trifluoromethyl)benzyl)-1H-indole-7-carboxamido)methyl)benzoic acid

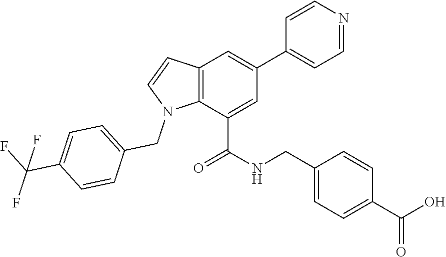 [SMILES: O=C(O)c1ccc(CNC(=O)c2cc(-c3ccncc3)cc3ccn(Cc4ccc(C(F)(F)F)cc4)c23)cc1]

Starting with pyridin-4-ylboronic acid (79.0 mg), the title product was obtained as a white solid (15 mg) by following the same procedure described for Example 75. LCMS (ESI): 530.5 [M+1]⁺.

¹HNMR (400 MHz, DMSO-d₆) δ 9.03 (t, J=6.0 Hz, 1H), 8.68-8.60 (m, 2H), 8.22 (d, J=1.8 Hz, 1H), 7.85 (d, J=8.0 Hz, 2H), 7.80-7.75 (m, 2H), 7.68 (d, J=3.2 Hz, 1H), 7.66 (d, J=1.8 Hz, 1H), 7.54 (d, J=8.1 Hz, 2H), 7.30 (d, J=8.0 Hz, 2H), 6.99 (d, J=8.0 Hz, 2H), 6.79 (d, J=3.1 Hz, 1H), 5.74 (s, 2H), 4.43 (d, J=5.8 Hz, 2H).

### EXAMPLE 81

### 4-((5-(4-cyanophenyl)-1-(4-(trifluoromethyl)benzyl)-1H-indole-7-carboxamido)methyl)benzoic acid

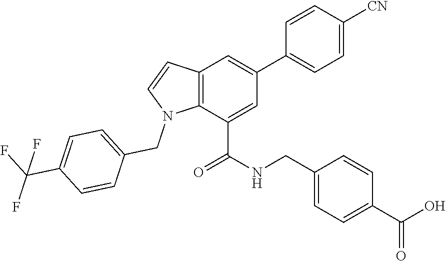 [SMILES: N#Cc1ccc(-c2cc(C(=O)NCc3ccc(C(=O)O)cc3)c3c(ccn3Cc3ccc(C(F)(F)F)cc3)c2)cc1]

Starting with (4-cyanophenyl)boronic acid (48.0 mg), the title product was obtained as a white solid (22 mg) by following the same procedure described for Example 75. LCMS (ESI): 554.5[M+1]⁺.

¹HNMR (400 MHz, DMSO-d₆) δ 9.02 (t, J=5.9 Hz, 1H), 8.16 (d, J=1.8 Hz, 1H), 7.97 (d, J=8.4 Hz, 2H), 7.93 (d, J=8.4 Hz, 2H), 7.85 (d, J=8.0 Hz, 2H), 7.67 (d, J=3.2 Hz, 1H), 7.61 (d, J=1.8 Hz, 1H), 7.54 (d, J=8.1 Hz, 2H), 7.30 (d, J=8.1 Hz, 2H), 6.99 (d, J=8.0 Hz, 2H), 6.78 (d, J=3.2 Hz, 1H), 5.74 (s, 2H), 4.43 (d, J=5.8 Hz, 2H).

### EXAMPLE 82

### 4-((5-(3-carbamoylphenyl)-1-(4-(trifluoromethyl)benzyl)-1H-indole-7-carboxamido)methyl)benzoic acid

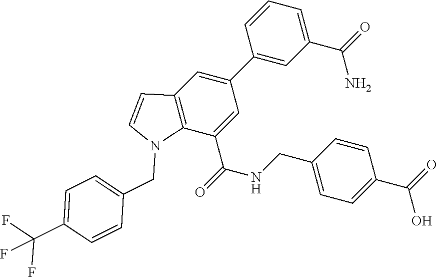 [SMILES: NC(=O)c1cccc(-c2cc(C(=O)NCc3ccc(C(=O)O)cc3)c3c(ccn3Cc3ccc(C(F)(F)F)cc3)c2)c1]

Starting with 3-aminocarbonyiphenylboronic acid, the title product was obtained as a white solid by following the same procedures as described in Example 75. LCMS (ESI): 572.2 [M+1]⁺.

¹HNMR (400 MHz, DMSO-d₆) δ 12.83 (s, 1H), 9.03 (t, J=5.9 Hz, 1H), 8.25 (t, J=1.8 Hz, 1H), 8.13-8.08 (m, 2H), 7.90 (dd, J=8.4, 6.5 Hz, 1H), 7.86-7.82 (m, 3H), 7.64 (d, J=3.2 Hz, 1H), 7.61 (d, J=1.8 Hz, 1H), 7.58-7.51 (m, 3H), 7.42 (s, 1H), 7.30 (d, J=8.0 Hz, 2H), 6.99 (d, J=8.0 Hz, 2H), 6.76 (d, J=3.2 Hz, 1H), 5.72 (s, 2H), 4.44 (d, J=5.9 Hz, 2H).

### EXAMPLE 83

### 4-((5-(3-sulfamoylphenyl)-1-(4-(trifluoromethyl)benzyl)-1H-indole-7-carboxamido)methyl)benzoic acid

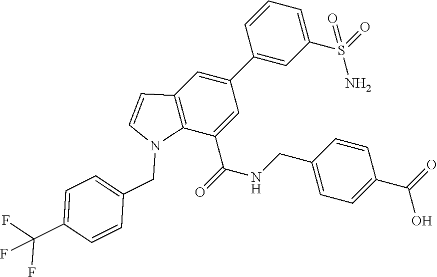 [SMILES: NS(=O)(=O)c1cccc(-c2cc(C(=O)NCc3ccc(C(=O)O)cc3)c3c(ccn3Cc3ccc(C(F)(F)F)cc3)c2)c1]

Starting with 3-(4,4,5,5-tetramethyl-1,3,2-dioxaborolan-2-yl)benzenesulfonamide, the title product was obtained as a white solid by following the same procedures described in Example 75. LCMS (ESI): 608.2 [M+1]⁺.

¹HNMR (400 MHz, DMSO-d₆) δ 12.79 (s, 1H), 9.07 (t, J=5.9 Hz, 1H), 8.19 (t, J=1.8 Hz, 1H), 8.08 (d, J=1.8 Hz, 1H), 7.97 (dt, J=7.8, 1.4 Hz, 1H), 7.84 (d, J=8.0 Hz, 2H), 7.79 (dt, J=7.8, 1.4 Hz, 1H), 7.70 7.64 (m, 2H), 7.60-7.51 (m, 3H), 7.41 (s, 2H), 7.30 (d, J=8.1 Hz, 2H), 7.00 (d, J=8.0 Hz, 2H), 6.78 (d, J=3.2 Hz, 1H), 5.72 (s, 2H), 4.43 (d, J=5.9 Hz, 2H).

### EXAMPLE 84

### 4-((5-(3-(methylsulfonyl)phenyl)-1-(4-(trifluoromethyl)benzyl)-1H-indole-7-carboxamido)methyl)benzoic acid

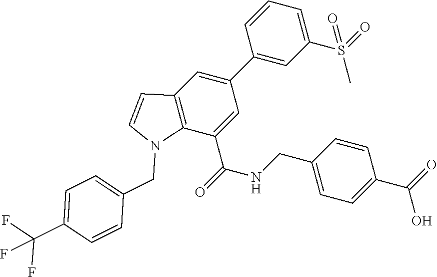 [SMILES: CS(=O)(=O)c1cccc(-c2cc(C(=O)NCc3ccc(C(=O)O)cc3)c3c(ccn3Cc3ccc(C(F)(F)F)cc3)c2)c1]

Starting with 3-(methylsulfonyl)benzeneboronic acid, the title product was obtained as a white solid by following the same procedure described in Example 75. MS (ESI): 607.2 [M+1]⁺.

¹HNMR (400 MHz, DMSO-d₆) δ 12.84 (s, 1H), 9.06 (t, J=5.9 Hz, 1H), 8.25 (d, J=1.9 Hz, 1H), 8.16 (d, J=1.7 Hz, 1H), 8.13-8.08 (m, 1H), 7.92-7.86 (m, 1H), 7.84 (d, J=8.0 Hz, 2H), 7.76 (t, J=7.8 Hz, 1H), 7.66 (d, J=3.2 Hz, 1H), 7.62 (d, J=1.8 Hz, 1H), 7.54 (d, J=8.1 Hz, 2H), 7.30 (d, J=8.0 Hz, 2H), 7.00 (d, J=8.0 Hz, 2H), 6.78 (d, J=3.1 Hz, 1H), 5.73 (s, 2H), 4.44 (d, J=5.8 Hz, 2H), 3.31 (s, 3H).

### EXAMPLE 85

### 4-((5-(3-(2-hydroxypropan-2-yl)phenyl)-1-(4-(trifluoromethyl)benzyl)-1H-indole-7-carboxamido)methyl)benzoic acid

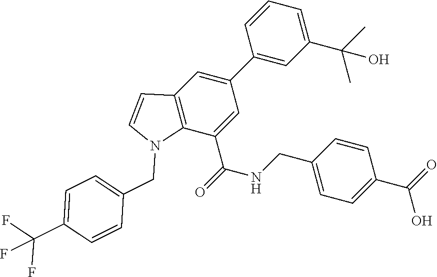 [SMILES: CC(C)(O)c1cccc(-c2cc(C(=O)NCc3ccc(C(=O)O)cc3)c3c(ccn3Cc3ccc(C(F)(F)F)cc3)c2)c1]

Starting with 3-(2-hydroxypropan-2-yl)phenylboronic acid, the title product was obtained as a white solid by following the same procedures as described for Example 75. LCMS (ESI): 587.2 [M+1]⁺.

¹HNMR (400 MHz, DMSO-d₆) δ 12.83 (s, 1H), 9.03 (t, J=6.0 Hz, 1H), 8.01 (d, J=1.7 Hz, 1H), 7.87-7.79 (m, 3H), 7.63 (d, J=3.2 Hz, 1H), 7.57-7.52 (m, 4H), 7.44 (dt, J=7.7, 1.5 Hz, 1H), 7.39 (t, J=7.6 Hz, 1H), 7.31 (d, J=8.0 Hz, 2H), 7.00 (d, J=8.1 Hz, 2H), 6.75 (d, J=3.1 Hz, 1H), 5.72 (s, 2H), 5.07 (s, 1H), 4.43 (d, J=5.9 Hz, 2H), 1.50 (s, 6H).

### EXAMPLE 86

### 4-((5-(2-hydroxyphenyl)-1-(4-(trifluoromethyl)benzyl)-1H-indole-7-carboxamido)methyl)benzoic acid

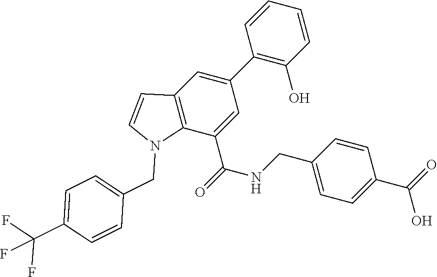 [SMILES: O=C(O)c1ccc(CNC(=O)c2cc(-c3ccccc3O)cc3ccn(Cc4ccc(C(F)(F)F)cc4)c23)cc1]

Starting with 2-hydroxyphenylboronic acid, the title product was obtained as a white solid by following the same procedures as described in Example 75. LCMS (ESI): 545.2 [M+1]⁺.

¹HNMR (400 MHz, CDCl₃) δ 7.94 (dd, J=7.9, 6.1 Hz, 2H), 7.86-7.82 (m, 1H), 7.46-7.41 (m, 3H), 7.32-7.24 (m, 2H), 7.25-7.13 (m, 4H), 7.01 (d, J=7.6 Hz, 2H), 6.98-6.89 (m, 2H), 6.69-6.64 (m, 1H), 5.68 (d, J=5.2 Hz, 2H), 4.37 (d, J=5.7 Hz, 2H).

### EXAMPLE 87

### 4-((5-(2-fluorophenyl)-1-(4-(trifluoromethyl)benzyl)-1H-indole-7-carboxamido)methyl)benzoic acid

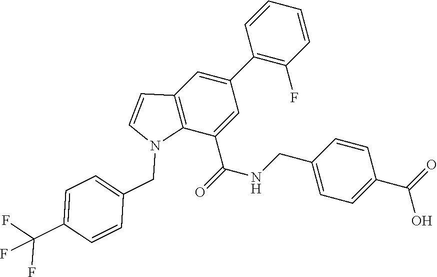 [SMILES: O=C(O)c1ccc(CNC(=O)c2cc(-c3ccccc3F)cc3ccn(Cc4ccc(C(F)(F)F)cc4)c23)cc1]

Starting with 2-fluorophenylboronic acid, the title product was obtained as a white solid by following the same procedure described in Example 75. MS (ESI): 547.2 [M+1]⁺.

¹HNMR (400 MHz, DMSO-d₆) δ 12.85 (s, 1H), 8.97 (t, J=5.9 Hz, 1H), 7.94 (d, J=1.8 Hz, 1H), 7.84 (d, J=8.1 Hz, 2H), 7.65 (dd, J=5.7, 2.4 Hz, 1H), 7.55 (d, J=8.1 Hz, 2H), 7.43-7.37 (m, 2H), 7.35-7.30 (m, 4H), 7.01 (d, J=8.1 Hz, 2H), 6.75 (d, J=3.2 Hz, 1H), 5.72 (s, 2H), 4.40 (d, J=5.9 Hz, 2H).

### EXAMPLE 88

### 4-((5-(3,5-dichlorophenyl)-1-(4-(trifluoromethyl)benzyl)-1H-indole-7-carboxamido)methyl)benzoic acid

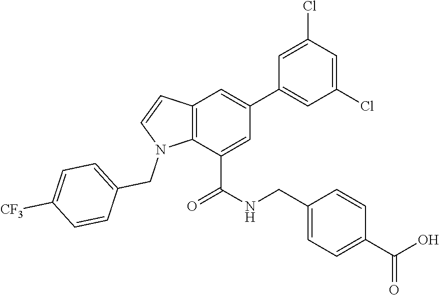 [SMILES: O=C(O)c1ccc(CNC(=O)c2cc(-c3cc(Cl)cc(Cl)c3)cc3ccn(Cc4ccc(C(F)(F)F)cc4)c23)cc1]

Starting with (3,5-dichlorophenyl)boronic acid, the title product was obtained as a white solid by following the same procedures as described in Example 75. LCMS (ESI): 599.3 [M+1]⁺. ¹H NMR (400 MHz, DMSO-d₆) δ 8.98 (t, J=5.9 Hz, 1H), 8.14 (s, 1H), 7.88-7.78 (m, 4H), 7.66 (d, J=3.2 Hz, 1H), 7.59 (d, J=1.7 Hz, 1H), 7.56-7.47 (m, 3H), 7.28 (d, J=8.0 Hz, 2H), 6.97 (d, J=8.0 Hz, 2H), 6.75 (d, J=3.1 Hz, 1H), 5.75 (s, 2H), 4.45 (d, J=5.9 Hz, 2H).

### EXAMPLE 89

### 4-((1-(4-(trifluoromethyl)benzyl)-5-(2-(trifluoromethyl)phenyl)-1H-indole-7-carboxamido)methyl)benzoic acid

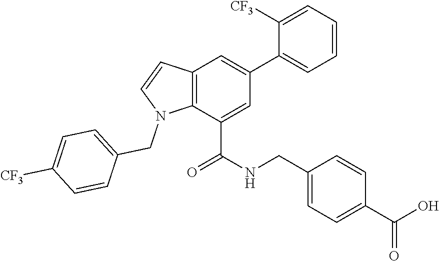 [SMILES: O=C(O)c1ccc(CNC(=O)c2cc(-c3ccccc3C(F)(F)F)cc3ccn(Cc4ccc(C(F)(F)F)cc4)c23)cc1]

Starting with (2-(trifluoromethyl)phenyl)boronic acid, the title product was obtained as a white solid by following the same procedures as described in Example 75. LCMS (ESI): 597.5 [M+1]⁺. ¹H NMR (400 MHz, DMSO-d₆) δ 8.92 (t, J=6.0 Hz, 1H), 7.86-7.82 (m, 3H), 7.74-7.69 (m, 2H), 7.66 (d, J=3.2 Hz, 1H), 7.61 (t, J=7.7 Hz, 1H), 7.57 (d, J=8.0 Hz, 2H), 7.51 (d, J=7.7 Hz, 1H), 7.29 (d, J=8.0 Hz, 2H), 7.21 (s, 1H), 7.06 (d, J=8.0 Hz, 2H), 6.72 (d, J=3.1 Hz, 1H), 5.72 (s, 2H), 4.38 (d, J=5.9 Hz, 2H).

### EXAMPLE 90

### 4-((5-(2-chlorophenyl)-1-(4-(trifluoromethyl)benzyl)-1H-indole-7-carboxamido)methyl)benzoic acid

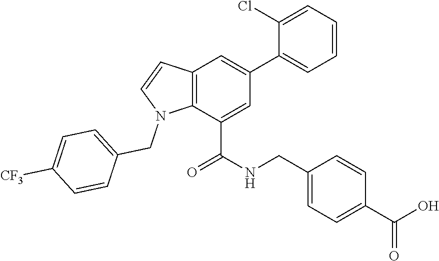 [SMILES: O=C(O)c1ccc(CNC(=O)c2cc(-c3ccccc3Cl)cc3ccn(Cc4ccc(C(F)(F)F)cc4)c23)cc1]

Starting with (2-chlorophenyl)boronic acid, the title product was obtained as a white solid by following the same procedures as described in Example 75. LCMS (ESI): 563.3 [M+1]. ¹H NMR (400 MHz, DMSO-d₆) δ 8.94 (t, J=6.0 Hz, 1H), 7.90-7.76 (m, 3H), 7.66 (d, J=3.2 Hz, 1H), 7.57 (m, 3H), 7.53-7.51 (m, 1H), 7.48-7.35 (m, 2H), 7.35-7.24 (m, 3H), 7.05 (d, J=8.0 Hz, 2H), 6.74 (d, J=3.1 Hz, 1H), 5.72 (s, 2H), 4.40 (d, J=5.8 Hz, 2H).

### EXAMPLE 91

### 4-((5-(4-chloro-3-fluorophenyl)-1-(4-(trifluoromethyl)benzyl)-1H-indole-7-carboxamido)methyl)benzoic

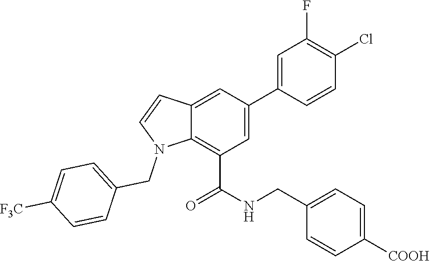 [SMILES: O=C(O)c1ccc(CNC(=O)c2cc(-c3ccc(Cl)c(F)c3)cc3ccn(Cc4ccc(C(F)(F)F)cc4)c23)cc1]

Starting with (4-chloro-3-fluorophenyl)boronic acid, the title product was obtained as a white solid by following the same procedures as described in Example 75. LCMS (ESI): 581.4 [M+1]⁺. ¹H NMR (400 MHz, DMSO-d₆) δ 8.99 (t, J=6.0 Hz, 1H), 8.10 (s, 1H), 7.91-7.78 (m, 3H), 7.72-7.61 (m, 3H), 7.58 (1, 1H), 7.53 (d, J=8.0 Hz, 2H), 7.30 (d, J=8.0 Hz, 2H), 6.98 (d, J=8.0 Hz, 2H), 6.75 (d, J=3.1 Hz, 1H), 5.74 (s, 2H), 4.44 (d, J=5.8 Hz, 2H).

### EXAMPLE 92

### 4-((5-(o-tolyl)-1-(4-(trifluoromethyl)benzyl)-1H-indole-7-carboxamido)methyl)benzoic acid

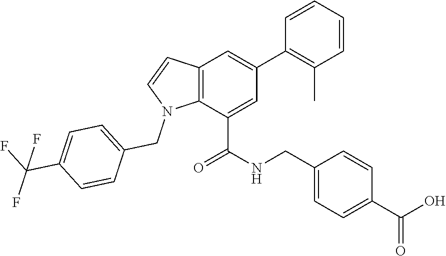 [SMILES: Cc1ccccc1-c1cc(C(=O)NCc2ccc(C(=O)O)cc2)c2c(ccn2Cc2ccc(C(F)(F)F)cc2)c1]

Starting with o-tolylboronic acid (45 mg), the title product was obtained as a white solid (40 mg) by following the same procedure described for Example 75. LCMS (ESI): 543.5[M+1]⁺.

¹HNMR (400 MHz, DMSO-d₆) δ 12.83 (s, 1H), 8.92 (t, J=6.0 Hz, 1H), 7.83 (d, J=8.1 Hz, 2H), 7.70 (d, J=1.7 Hz, 1H), 7.63 (d, J=3.2 Hz, 1H), 7.56 (d, J=8.1 Hz, 2H), 7.34-7.23 (m, 6H), 7.20 (d, J=1.7 Hz, 1H), 7.06 (d, J=8.0 Hz, 2H), 6.70 (d, J=3.2 Hz, 1H), 5.72 (s, 2H), 4.39 (d, J=5.9 Hz, 2H), 2.29 (s, 3H).

### EXAMPLE 93

### 4-((5-(2-aminophenyl)-1-(4-(trifluoromethyl)benzyl)-1H-indole-7-carboxamido)methyl)benzoic acid

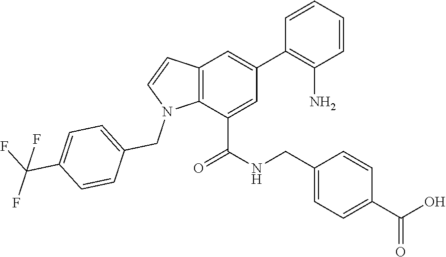 [SMILES: Nc1ccccc1-c1cc(C(=O)NCc2ccc(C(=O)O)cc2)c2c(ccn2Cc2ccc(C(F)(F)F)cc2)c1]

Starting with (2-aminophenyl)boronic acid (45 mg), the title product was obtained as a white solid by following the same procedure described for Example 75. LCMS (ESI): 544.5[M+1]⁺.

¹HNMR (400 MHz, DMSO-d₆) δ 8.93 (t, J=6.0 Hz, 1H), 7.84 (d, J=8.2 Hz, 2H), 7.73 (d, J=1.7 Hz, 1H), 7.62 (d, J=3.2 Hz, 1H), 7.58 (d, J=8.0 Hz, 2H), 7.34-7.25 (m, 3H), 7.08-7.02 (m, 7.6, 1.6 Hz, 4H), 6.76 (d, J=8.0, 1H), 6.70 (d, J=3.1 Hz, 1H), 6.64 (t, J=7.4, 1.2 Hz, 1H), 5.72 (s, 2H), 4.88 (brs, 2H), 4.39 (d, J=5.9 Hz, 2H).

### EXAMPLE 94

### 4-((5-(3-chloro-4-fluorophenyl)-1-(4-(trifluoromethyl)benzyl)-1H-indole-7-carboxamido)methyl)benzoic

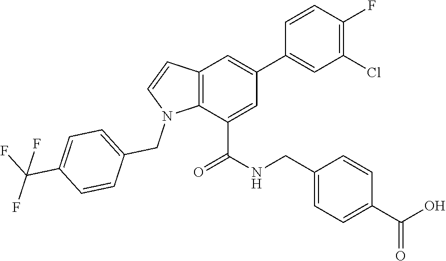 [SMILES: O=C(O)c1ccc(CNC(=O)c2cc(-c3ccc(F)c(Cl)c3)cc3ccn(Cc4ccc(C(F)(F)F)cc4)c23)cc1]

Starting with (3-chloro-4-fluorophenyl)boronic acid, the title product was obtained as a white solid by following the same procedure described for Example 75. LCMS (ESI): 581.5[M+1]⁺.

¹HNMR (400 MHz, DMSO-d₆) δ 8.97 (t, J=6.0 Hz, 1H), 8.06 (d, J=1.9 Hz, 1H), 7.95 (dd, J=7.1, 2.4 Hz, 1H), 7.84 (d, J=7.8 Hz, 2H), 7.76-7.73 (m, 1H), 7.65 (d, J=3.2 Hz, 1H), 7.54-7.49 (m, 4H), 7.29 (d, J=7.7 Hz, 2H), 6.98 (d, J=8.0 Hz, 2H), 6.74 (d, J=3.2 Hz, 1H), 5.74 (s, 2H), 4.43 (d, J=5.8 Hz, 2H).

### EXAMPLE 95

### 4-((5-(3-chloro-5-fluorophenyl)-1-(4-(trifluoromethyl)benzyl)-1H-indole-7-carboxamido)methyl)benzoic acid

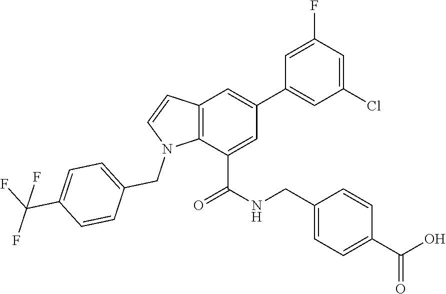 [SMILES: O=C(O)c1ccc(CNC(=O)c2cc(-c3cc(F)cc(Cl)c3)cc3ccn(Cc4ccc(C(F)(F)F)cc4)c23)cc1]

Starting with (3-chloro-5-fluorophenyl)boronic acid (38 mg), the title product was obtained as a white solid (12 mg) by following the same procedure described for Example 75. LCMS (ESI): 581.5[M+1]⁺.

¹HNMR (400 MHz, DMSO-d₆) δ 12.84 (s, 1H), 8.97 (t, J=5.9 Hz, 1H), 8.14 (d, J=1.8 Hz, 1H), 7.84 (d, J=8.1 Hz, 2H), 7.71 (d, J=1.7 Hz, 1H), 7.67 (d, J=3.2 Hz, 1H), 7.63 (d, J=10.1, 1H), 7.59 (d, J=1.9 Hz, 1H), 7.53 (d, J=8.1 Hz, 2H), 7.38 (d, J=8.6, 1H), 7.29 (d, J=8.0 Hz, 2H), 6.98 (d, J=8.0 Hz, 2H), 6.75 (d, J=3.2 Hz, 1H), 5.75 (s, 2H), 4.45 (d, J=5.8 Hz, 2H).

### EXAMPLE 96

### 4-((5-(4-fluorophenyl)-1-(4-(trifluoromethyl)benzyl)-1H-indazole-7-carboxamido)methyl)benzoic acid

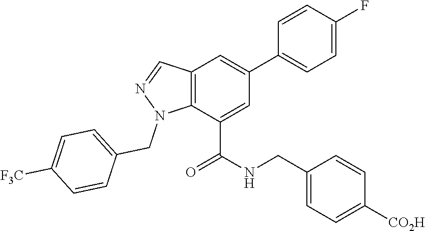 [SMILES: O=C(O)c1ccc(CNC(=O)c2cc(-c3ccc(F)cc3)cc3cnn(Cc4ccc(C(F)(F)F)cc4)c23)cc1]

### Step 1 methyl 5-bromo-1-(4-(trifluoromethyl)benzyl)-1H-indazole-7-carboxylate

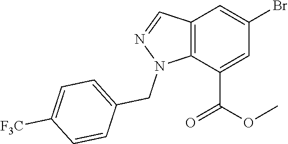 [SMILES: COC(=O)c1cc(Br)cc2cnn(Cc3ccc(C(F)(F)F)cc3)c12]

To a mixture of methyl 5-bromo-1H-indole-7-carboxylate (5.0 g) and Cs₂CO₃ (21.5 g) in anhydrous DMF (100 mL) at 0° C. was added 1-(bromomethyl)-4-(trifluoromethyl)benzene (5.7 g). The reaction mixture was then warmed to rt and stirred for 6 hrs. The reaction mixture was poured into 100 mL ice-water, extracted 3×50 mL EtOAc. The reaction mixture was poured into aqueous NH4C1 solution.

The solid was collected by filtration and dried, then dissolved in (EA/PE, 60/180 mL,. The solution was filtered through a pad of celite and concentrated to obtained 5.1 g of the crude title product as a white solid. LCMS (ESI): 413 [M+1]⁺.

The solid was collected by filtration and dried, then purified by silica gel chromatography (EA/PE, ⅓) to obtained 5.1 g white Isomer 1 as the title product and Isomer 2 as a white solid. LCMS (ES+): 413 [M+1]

Isomer 1: R_{f}=0.5 (EA/PE=⅕). ¹H NMR (400 MHz, CDCl₃) δ 8.19-8.03 (m, 2H), 7.99 (d, 1H), 7.49 (d, 2H), 7.08 (d, 2H), 6.06 (s, 2H), 3.82 (s, 3H).

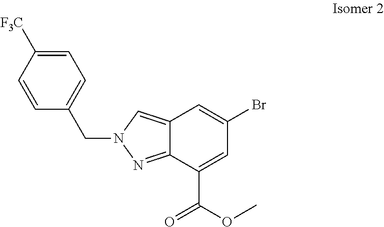 [SMILES: COC(=O)c1cc(Br)cc2cn(Cc3ccc(C(F)(F)F)cc3)nc12]

Isomer 2: R_{f}=0.2 (EA/PE=⅕). ¹H NMR (400 MHz, CDCl₃) δ 8.15 (d, 1H), 8.01 (d, 1H), 7.92 (s, 1H), 7.63 (d, 2H), 7.41 (d, 2H), 5.76 (s, 2H), 4.02 (s, 3H).

### Step 2 4-((5-(4-fluorophenyl)-1-(4-(trifluoromethyl)benzyl)-1H-indazole-7-carboxamido)methyl)benzoic acid

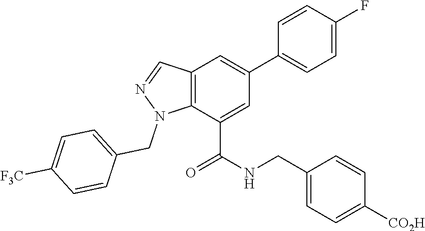 [SMILES: O=C(O)c1ccc(CNC(=O)c2cc(-c3ccc(F)cc3)cc3cnn(Cc4ccc(C(F)(F)F)cc4)c23)cc1]

Starting with the product of Step 1 and 4-fluorophenylboronic acid, the title product was obtained as a white solid by following the same procedures as described in Step 2 to Step 5 of Example 75. LCMS (ESI): 548 [M+1]⁺.

¹HNMR (400 MHz, CDCl₃) δ 8.21 (s, 1H), 8.01 (m, 3H), 7.60 (s, 1H), 7.56-7.42 (m, 5H), 7.15 (t, 2H), 7.09 (d, 2H), 6.09 (s, 1H), 6.00 (s, 2H), 4.52 (d, 2H), 3.92 (s, 1H).

### EXAMPLE 97

### 4-((5-phenyl-1-(4-(trifluoromethyl)benzyl)-1H-indazole-7-carboxamido)methyl)benzoic acid

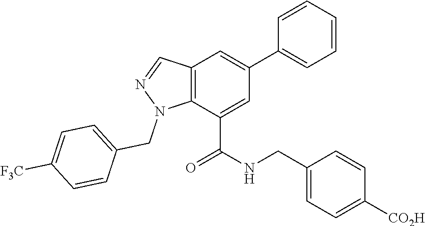 [SMILES: O=C(O)c1ccc(CNC(=O)c2cc(-c3ccccc3)cc3cnn(Cc4ccc(C(F)(F)F)cc4)c23)cc1]

Starting with phenylboronic acid, the title product was obtained as a white solid by following the same procedures as described in Example 96. LCMS (ESI): 530 [M+1]⁺.

¹HNMR (400 MHz, CDCl₃) δ 8.22 (s, 1H), 8.05-8.00 (m, 3H), 7.64 (d, 1H), 7.61-7.52 (m, 3H), 7.46 (s, 4H), 7.37 (s, 2H), 7.11 (s, 2H), 6.10 (s, 1H), 6.00 (s, 1H), 4.52 (s, 1H), 3.92 (s, 1H).

### EXAMPLE 98

### 4-((5-(3-cyanophenyl)-1-(4-(trifluoromethyl)benzyl)-1H-indazole-7-carboxamido)methyl)benzoic acid

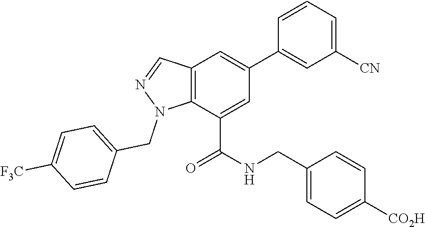 [SMILES: N#Cc1cccc(-c2cc(C(=O)NCc3ccc(C(=O)O)cc3)c3c(cnn3Cc3ccc(C(F)(F)F)cc3)c2)c1]

Starting with (3-cyanophenyl)boronic acid, the title product was obtained as a white solid by following the same procedures as described in Example 96. LCMS (ESI): 555 [M+1]⁺.

¹HNMR (400 MHz, DMSO-d₆) δ 9.27 (t, 1H), 8.42-8.33 (m, 2H), 8.33-8.27 (m, 1H), 8.15 (d, 1H), 7.94 (d, 1H), 7.84 (d, 3H), 7.70 (dd, 1H), 7.53 (d, 2H), 7.34 (d, 2H), 7.05 (d, 2H), 5.31 (s, 2H), 4.53 (d, 2H).

### EXAMPLE 99

### 4-((5-(3,5-difluorophenyl)-1-(4-(trifluoromethyl)benzyl)-1H-indazole-7-carboxamido)methyl)benzoic acid

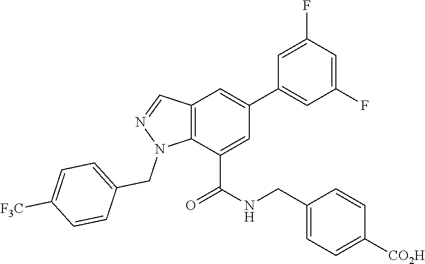 [SMILES: O=C(O)c1ccc(CNC(=O)c2cc(-c3cc(F)cc(F)c3)cc3cnn(Cc4ccc(C(F)(F)F)cc4)c23)cc1]

Starting with (3,5-difluorophenyl)boronic acid, the title product was obtained as a white solid by following the same procedures as described in Example 96. LCMS (ESI): 566 [M+1]⁺.

¹HNMR (400 MHz, DMSO-d₆) δ 9.25 (t, 1H), 8.42-8.31 (m, 2H), 7.93 (t, 1H), 7.85 (d, 2H), 7.62-7.56 (m, 2H), 7.53 (d, 2H), 7.34 (d, 2H), 7.25 (tt, 1H), 7.05 (d, 2H), 5.96 (s, 2H), 4.53 (d, 2H).

### EXAMPLE 100

### (S)-4-(1-(5-phenyl-1-(4-(trifluoromethyl)benzyl)-1H-indazole-7-carboxamido)ethyl)benzoic acid

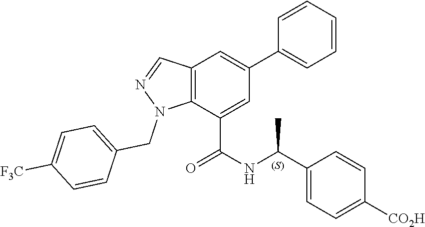

Starting with phenylboronic acid and (S)-methyl 4-(1-aminoethyl)benzoate, the title product was obtained as a white solid by following the same procedures as described in Example 96. LCMS (ESI): 544 [M+1]⁺.

### EXAMPLE 101

### (S)-4-(1-(5-(3,5-difluorophenyl)-1-(4-(trifluoromethyl)benzyl)-1H-indazole-7-carboxamido)ethyl)benzoic acid

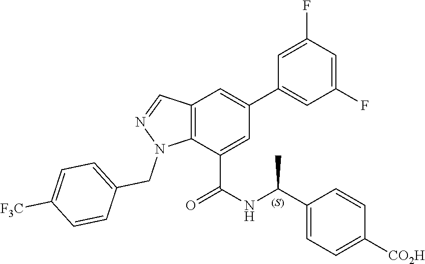

Starting with (3,5-difluorophenyl)boronic acid and (S)-methyl 4-(1-aminoethyl)benzoate, tThe title product was obtained as a white solid by following the same procedures as described in Example 96. LCMS (ESI): 580 [M+1]⁺.

### EXAMPLE 102

### 4-((1-(3-chlorobenzyl)-5-(3-cyanophenyl)-1H-indole-7-carboxamido)methyl)benzoic acid

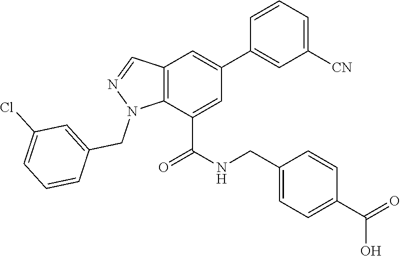 [SMILES: N#Cc1cccc(-c2cc(C(=O)NCc3ccc(C(=O)O)cc3)c3c(cnn3Cc3cccc(Cl)c3)c2)c1]

Starting with 1-(bromomethyl)-3-chlorobenzene and (3-cyanophenyl)boronic acid, the title product was obtained as a white solid by following the same procedures described in Example 75. LCMS (ESI): 622.12 [M+1]⁺.

¹HNMR (400 MHz, DMSO-d₆) δ 9.07 (d, J=5.9 Hz, 1H), 8.22 (s, 1H), 8.17-8.05 (m, 2H), 7.85 (d, J=8.0 Hz, 2H), 7.79 (d, J=7.7 Hz, 1H), 7.73-7.59 (m, 3H), 7.36-7.20 (m, 4H), 6.90 (s, 1H), 6.85-6.69 (m, 2H), 5.63 (s, 2H), 4.47 (d, J=5.6 Hz, 2H).

### EXAMPLE 103

### 4-((1-(3-chlorobenzyl)-4-(phenylamino)-1H-indole-7-carboxamido)methyl)benzoic acid

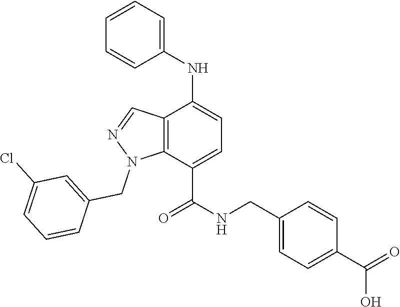 [SMILES: O=C(O)c1ccc(CNC(=O)c2ccc(Nc3ccccc3)c3cnn(Cc4cccc(Cl)c4)c23)cc1]

Starting with 1-(bromomethyl)-3-chlorobenzene (70 mg) and aniline (70 mg), the title product was obtained as a white solid (25 mg) by following the same procedure described in Example 64. LCMS (ESI): 510.2 [M+1]⁺.

¹HNMR (400 MHz, DMSO-d₆) δ 8.76 (t, J=6.0 Hz, 1H), 8.25 (s, 1H), 7.84 (d, J=8.0 Hz, 2H), 7.43 (d, J=3.2 Hz, 1H), 7.32-7.18 (m, 9H), 6.91 (t, J=7.2 Hz, 1H), 6.89-6.85 (m, 2H), 6.81 (dd, J=12.3, 7.7 Hz, 2H), 5.61 (s, 2H), 4.42 (d, J=5.9 Hz, 2H).

### EXAMPLE 104

### 4-((1-(3-chlorobenzyl)-5-(pyridin-3-yl)-1H-indole-7-carboxamido)methyl)benzoic acid

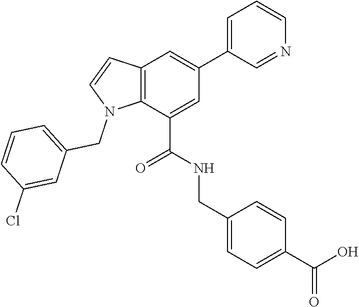 [SMILES: O=C(O)c1ccc(CNC(=O)c2cc(-c3cccnc3)cc3ccn(Cc4cccc(Cl)c4)c23)cc1]

Starting with 1-(bromomethyl)-3-chlorobenzene and pyridin-3-ylboronic acid, the title product was obtained as a white solid by following the same procedures in Example 75. LCMS (ESI): 596.2 [M+1]⁺.

¹H NMR (400 MHz, DMSO-d₆) δ 12.85 (s, 1H), 9.08 (t, J=5.9 Hz, 1H), 8.97 (d, J=2.4 Hz, 1H), 8.55 (dd, J=4.8, 1.6 Hz, 1H), 8.14 (dt, J=8.1, 2.0 Hz, 1H), 8.10 (d, J=1.8 Hz, 1H), 7.87 (d, J=8.0 Hz, 2H), 7.65 (d, J=3.2 Hz, 1H), 7.59 (d, J=1.9 Hz, 1H), 7.49 (dd, J=7.9, 4.8 Hz, 1H), 7.34 (d, J=8.0 Hz, 2H), 7.31-7.21 (m, 2H), 6.92 (d, J=2.1 Hz, 1H), 6.82 (d, J=7.5 Hz, 1H), 6.75 (d, J=3.2 Hz, 1H), 5.64 (s, 2H), 4.48 (d, J=5.8 Hz, 2H).

### EXAMPLE 105

### 4-((1-(3-chlorobenzyl)-5-phenyl-1H-indole-7-carboxamido)methyl)benzoic acid

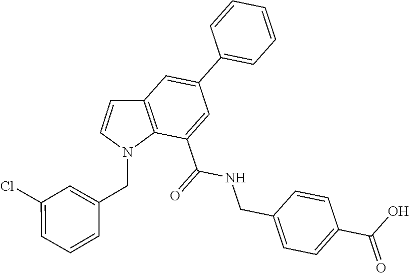 [SMILES: O=C(O)c1ccc(CNC(=O)c2cc(-c3ccccc3)cc3ccn(Cc4cccc(Cl)c4)c23)cc1]

Starting with 1-(bromomethyl)-3-chlorobenzene and phenylboronic acid, the title product was obtained as a white solid by following the same procedure described in Example 75. MS (ESI): 595.2 [M+1]⁺.

¹HNMR (400 MHz, DMSO-d₆) δ 13.32-12.42 (brs, 1H), 9.09 (d, J=7.1 Hz, 1H), 8.02 (d, J=7.9 Hz, 1H), 7.87 (d, J=7.8 Hz, 2H), 7.74 (d, J=7.8 Hz, 2H), 7.61 (d, J=7.8 Hz, 1H), 7.56 (d, J=7.9 Hz, 1H), 7.48 (d, J=7.8 Hz, 2H), 7.35 (d, J=7.9 Hz, 3H), 7.29-7.23 (m, 2H), 6.94 (d, J=8.0 Hz, 1H), 6.83 (d, J=7.6 Hz, 1H), 6.73 (d, J=8.8 Hz, 1H), 5.63 (d, J=7.6 Hz, 2H), 4.48 (t, J=6.4 Hz, 2H).

### EXAMPLE 106

### 4-((1-(3-chlorobenzyl)-5-(3,5-difluorophenyl)-1H-indole-7-carboxamido)methyl)benzoic acid

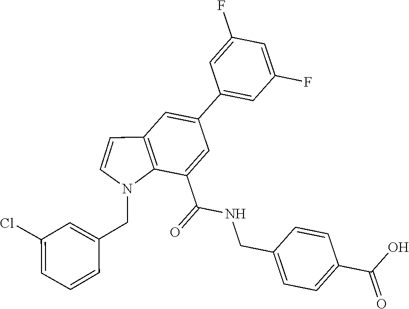 [SMILES: O=C(O)c1ccc(CNC(=O)c2cc(-c3cc(F)cc(F)c3)cc3ccn(Cc4cccc(Cl)c4)c23)cc1]

Starting with 1-(bromomethyl)-3-chlorobenzene and (3,5-difluorophenyl)boronic acid, the title product was obtained as a white solid by following the same procedures as described for Example 75. LCMS (ESI): 531.1 [M+1]⁺.

¹HNMR (400 MHz, DMSO-d₆) δ 12.87 (s, 1H), 9.06 (t, J=5.9 Hz, 1H), 8.13 (d, J=1.8 Hz, 1H), 7.87 (d, J=8.0 Hz, 2H), 7.65 (d, J=3.2 Hz, 1H), 7.60 (d, J=1.9 Hz, 1H), 7.54-7.48 (m, 2H), 7.33 (d, J=8.0 Hz, 2H), 7.29-7.22 (m, 2H), 7.19 (tt, J=9.2, 2.3 Hz, 1H), 6.90 (d, J=2.1 Hz, 1H), 6.79 (d, J=7.5 Hz, 1H), 6.74 (d, J=3.1 Hz, 1H), 5.64 (s, 2H), 4.48 (d, J=5.8 Hz, 2H).

### EXAMPLE 107

### 4-((1-(3-chlorobenzyl)-5-(4-fluorophenyl)-1H-indole-7-carboxamido)methyl)benzoic acid

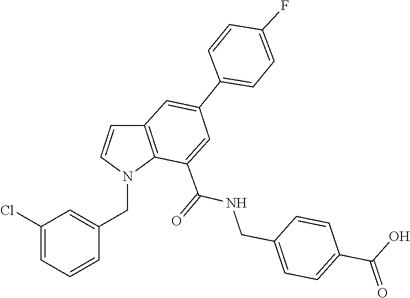 [SMILES: O=C(O)c1ccc(CNC(=O)c2cc(-c3ccc(F)cc3)cc3ccn(Cc4cccc(Cl)c4)c23)cc1]

Starting with 1-(bromomethyl)-3-chlorobenzene and (4-fluorophenyl)boronic acid, the title product was obtained as a white solid by following the same procedures as described in Example 75. LCMS (ESI): 513.2 [M+1]⁺.

¹HNMR (400 MHz, DMSO-d₆) δ 12.84 (s, 1H), 9.07 (t, J=6.0 Hz, 1H), 8.00 (d, J=1.8 Hz, 1H), 7.87 (d, J=8.1 Hz, 2H), 7.81-7.74 (m, 2H), 7.62 (d, J=3.2 Hz, 1H), 7.51 (d, J=1.8 Hz, 1H), 7.34 (s, 1H), 7.35 7.25 (m, 4H), 7.24 (t, J=7.7 Hz, 1H), 6.91 (d, J=2.0 Hz, 1H), 6.82 (d, J=7.5 Hz, 1H), 6.72 (d, J=3.2 Hz, 1H), 5.63 (s, 2H), 4.47 (d, J=5.9 Hz, 2H).

### EXAMPLE 108

### (S)-4-(1-(1-(3-chlorobenzyl)-5-(3-cyanophenyl)-1H-indole-7-carboxamido)ethyl)benzoic acid

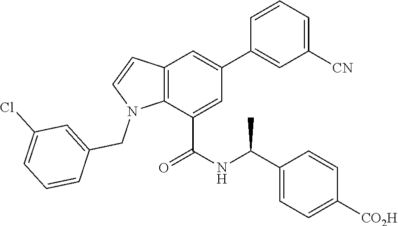 [SMILES: C[C@H](NC(=O)c1cc(-c2cccc(C#N)c2)cc2ccn(Cc3cccc(Cl)c3)c12)c1ccc(C(=O)O)cc1]

Starting with 1-(bromomethyl)-3-chlorobenzene, (3-cyanophenyl)boronic acid and (S)-Methyl 4-(1-aminoethyl)benzoate, the title product was obtained as a white solid (26 mg) by following the same procedure described for Example 75. LCMS (ESI): 534.5[M+1]⁺.

¹HNMR (400 MHz, DMSO-d₆) δ 12.85 (s, 1H), 9.00 (d, J=7.6 Hz, 1H), 8.23 (d, J=1.9 Hz, 1H), 8.16-8.04 (m, 2H), 7.88 (d, J=8.1 Hz, 2H), 7.81 (d, J=7.6 Hz, 1H), 7.71 (t, J=7.8 Hz, 1H), 7.63 (d, J=3.2 Hz, 1H), 7.55 (d, J=1.9 Hz, 1H), 7.46 (d, J=8.1 Hz, 2H), 7.30-7.18 (m, 2H), 6.87 (d, J=2.0 Hz, 1H), 6.78 (d, J=7.6 Hz, 1H), 6.73 (d, J=3.2 Hz, 1H), 5.63-5.49 (m, 2H), 5.07-5.01 (m, J=7.1 Hz, 1H).

### EXAMPLE 109

### (S)-4-(1-(1-(3-chlorobenzyl)-5-(pyridin-3-yl)-1H-indole-7-carboxamido)ethyl)benzoic acid

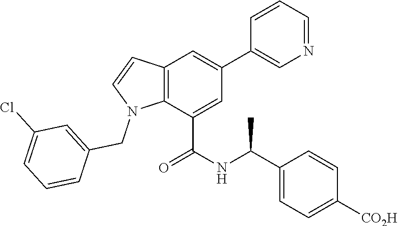 [SMILES: C[C@H](NC(=O)c1cc(-c2cccnc2)cc2ccn(Cc3cccc(Cl)c3)c12)c1ccc(C(=O)O)cc1]

Starting with 1-(bromomethyl)-3-chlorobenzene, (S)-Methyl 4-(1-aminoethyl)benzoate and pyridin-3-ylboronic acid, the title product was obtained as a white solid by following the same procedure described for Example 75. LCMS (ESI): 510.5[M+1]⁺.

¹HNMR (400 MHz, DMSO-d₆) δ 12.84 (s, 1H), 9.03-8.97 (m, 2H), 8.56 (d, J=4.7 Hz, 1H), 8.15 (d, J=8.0 Hz, 1H), 8.09 (d, J=1.8 Hz, 1H), 7.89 (d, J=8.1 Hz, 2H), 7.63 (d, J=3.2 Hz, 1H), 7.56-7.49 (m, 2H), 7.46 (d, J=8.1 Hz, 2H), 7.29-7.24 (m, 1H), 7.22 (t, J=7.8 Hz, 1H), 6.88 (d, J=2.1 Hz, 1H), 6.79 (d, J=7.6 Hz, 1H), 6.74 (d, J=3.1 Hz, 1H), 5.65-5.47 (m, 2H), 5.12-5.06 (m, J=7.2 Hz, 1H).

### EXAMPLE 110

### (S)-4-(1-(1-(3-chlorobenzyl)-5-phenyl-1H-indole-7-carboxamido)ethyl)benzoic acid

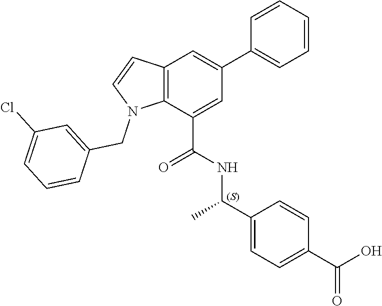

Starting with 1-(bromomethyl)-3-chlorobenzene, (S)-Methyl 4-(1-aminoethyl)benzoate and phenylboronic acid, the title product was obtained as a white solid by following the same procedure described for Example 75. LCMS (ESI): 509.5[M+1]⁺.

¹HNMR (400 MHz, DMSO-d₆) δ 12.84 (s, 1H), 9.03 (d, J=7.7 Hz, 1H), 8.01 (d, J=1.8 Hz, 1H), 7.89 (d, J=8.2 Hz, 2H), 7.78-7.71 (m, 2H), 7.60 (d, J=3.2 Hz, 1H), 7.54-7.43 (m, 5H), 7.35 (t, J=7.4 Hz, 1H), 7.29-7.25 (m, 1H), 7.22 (t, J=7.8 Hz, 1H), 6.88 (d, J=2.0 Hz, 1H), 6.80 (d, J=7.6 Hz, 1H), 6.71 (d, J=3.1 Hz, 1H), 5.65-5.47 (m, 2H), 5.12-5.06 (m, J=7.2 Hz, 1H).

### EXAMPLE 111

### (S)-4-(1-(1-(3-chlorobenzyl)-5-(3,5-difluorophenyl)-1H-indole-7-carboxamido)ethyl)benzoic acid

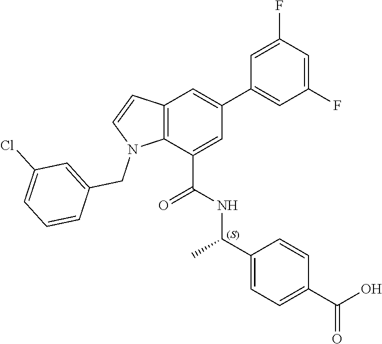

Starting with 1-(bromomethyl)-3-chlorobenzene, (S)-Methyl 4-(1-aminoethyl)benzoate and (3,5-difluorophenyl)boronic acid, the title product was obtained as a white solid by following the same procedure described for Example 75. LCMS (ESI): 545.5[M+1]⁺.

¹HNMR (400 MHz, DMSO-d₆) δ 12.85 (s, 1H), 8.98 (d, J=7.6 Hz, 1H), 8.11 (d, J=1.8 Hz, 1H), 7.88 (d, J=8.2 Hz, 2H), 7.63 (d, J=3.2 Hz, 1H), 7.57-7.49 (m, 3H), 7.46 (d, J=8.2 Hz, 2H), 7.29-7.25 (m, 1H), 7.24-7.17 (m, 2H), 6.86 (d, J=2.1 Hz, 1H), 6.78 (d, J=7.6 Hz, 1H), 6.72 (d, J=3.1 Hz, 1H), 5.63-5.51 (m, 2H), 5.11-5.06 (m, 1H).

### EXAMPLE 112

### (+) 6-(6-bromo-3-((4-(trifluoromethyl)phenyl)thio)-1H-indole-4-carboxamido)spiro[3.3]heptane-2-carboxylic acid

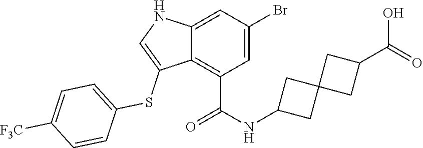 [SMILES: O=C(NC1CC2(C1)CC(C(=O)O)C2)c1cc(Br)cc2[nH]cc(Sc3ccc(C(F)(F)F)cc3)c12]

### Step 1 methyl 5-bromo-1-(4-(trifluoromethyl)benzyl)-1H-indole-7-carboxylate

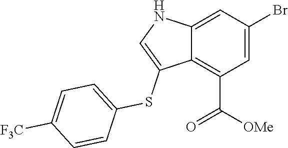 [SMILES: COC(=O)c1cc(Br)cc2[nH]cc(Sc3ccc(C(F)(F)F)cc3)c12]

A mixture of methyl 6-bromo-1H-indole-4-carboxylate (3.9 mmol), 4-(trifluoromethyl)benzenethiol (4.3 mmol) and tBuOOH (4.29 mmol) were dissolved in MeCN (20.0 mL) at 60° C. in a flask, then iodine (0.10 mmol, 10 mol %) was added. The reaction mixture was stirred for 1 h, quenched by the addition of saturated aq. Na₂S20₃ (5 mL) and then extracted with EtOAc (2×60 mL). The combined organic layer was dried (MgSO4), filtered and concentrated under vacuum and the crude product was purified by column chromatography using petroleum ether/ethyl acetate as eluent to to afford a pale-yellow solid (1.59 g).

### LCMS (ESI): 430.2 [M+1]⁺.

### Step 2 6-bromo-3-((4-(trifluoromethyl)phenyl)thio)-1H-indole-4-carboxylic acid

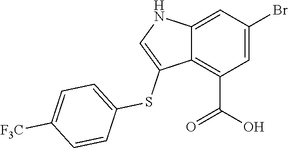 [SMILES: O=C(O)c1cc(Br)cc2[nH]cc(Sc3ccc(C(F)(F)F)cc3)c12]

To a solution of the product of Step 1 (520 mg, 1.21 mmol) in MeOH/H₂O (10 mL/2.0 mL) was added LiOH—H₂O (254 mg, 6.06 mmol) at rt. The reaction mixture was stirred at 60° C. for 1 h. After being cooled to 0° C., the reaction mixture was treated with 6 N HCl to pH≈6. The reaction mixture was extracted 3 x 30 mL EtOAc. The combined organic layer washed with 20 mL saturated brine, dried over anhydrous Na₂SO₄, concentrated. The residue was purified by silica gel column chromatography (DCM/MeOH: from 100:0 to 10:1) to afford the title compound as a white solid (480 mg). LCMS (ESI): 416 [M+1]⁺.

### Step 3 (±)methyl 6-aminospiro[3.3]heptane-2-carboxylate hydrochloride

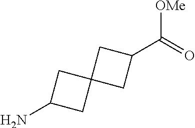 [SMILES: COC(=O)C1CC2(CC(N)C2)C1]

To a solution of methyl 6-((tert-butoxycarbonyl)amino)spiro[3.3]heptane-2-carboxylate in THF (10 ml) was added 4M HCl in dioxane (1.0 ml), After stirring overnight, the mixture was concentrated to give the title compound without further putification to afford as a white solid (0.52 g). LCMS (ESI): 170 [M+1]⁺.

### Step 4 (±)methyl 6-(6-bromo-3-((4-(trifluoromethyl)phenyl)thio)-1H-indole-4-carboxamido)spiro [3.3]heptane-2-carboxylate

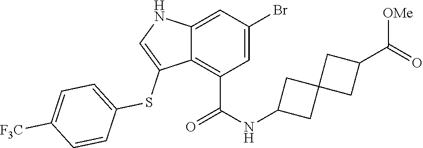 [SMILES: COC(=O)C1CC2(CC(NC(=O)c3cc(Br)cc4[nH]cc(Sc5ccc(C(F)(F)F)cc5)c34)C2)C1]

To a mixture of the product of Step 2 (416 mg), the product of Step 32 (266.5 mg) and HATU (456 mg) in anhydrous DMF (10 mL) at 0° C. was added DIPEA (0.52 mL). After stirring at rt overnight, the reaction mixture was quenched with 100 mL water and extracted 3×100 mL EtOAc. The combined organic layer was washed with 100 mL saturated brine, dried over anhydrous Na₂SO₄ and concentrated. The residue was purified by silica gel column chromatography (petroleum ether/ethyl acetate: from 100:0 to 5:1) to afford the title compound as a white solid (0.52 g). LCMS (ESI): 567 [M+1]⁺.

### Step 5 (±) 6-(6-bromo-3-((4-(trifluoromethyl)phenyl)thio)-1H-indole-4-carboxamido)spiro-[3.3]heptane-2-carboxylic acid

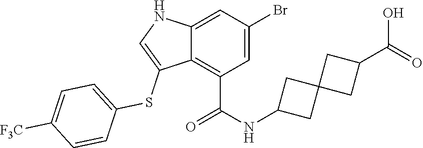 [SMILES: O=C(NC1CC2(C1)CC(C(=O)O)C2)c1cc(Br)cc2[nH]cc(Sc3ccc(C(F)(F)F)cc3)c12]

To a solution of the product of Step 4 (57 mg) in MeOH/H₂O (5 mL/0.5 mL) was added LiOH—H₂O (20 mg). The reaction mixture was stirred at 40° C. for 12 h. After being cooled to 0° C., the mixture was treated with 6 N HCl to pH≈6. The reaction mixture was then extracted 3×20 mL EtOAc. The combined organic layer was washed with 20 mL saturated brine, dried over anhydrous Na₂SO₄ and concentrated. The residue was purified by silica gel column chromatography (DCM/MeOH=10:1) to afford the title compound as a white solid (40 mg). LCMS (ESI): 553.2 [M+1]⁺.

¹H NMR (400 MHz, DMSO-d₆) δ 12.12 (s, 1H), 8.25 (d, J=7.1 Hz, 1H), 7.86 (s, 1H), 7.75 (d, J=1.8 Hz, 1H), 7.49 (d, J=8.3 Hz, 2H), 7.09 (d, J=1.8 Hz, 1H), 7.05 (d, J=8.3 Hz, 2H), 3.99-3.92 (m, 1H), 2.90 2.83 (m, 1H), 2.26-2.08 (m, 3H), 2.02-1.97 (m, 2H), 1.94-1.90 (m, 1H), 1.73-1.69 (m, 1H), 1.66-1.62 (m, 1H).

### EXAMPLE 113

### (±) 6-(6-phenyl-3-((4-(trifluoromethyl)phenyl)thio)-1H-indole-4-carboxamido)spiro[3.3]heptane-2-carboxylic acid

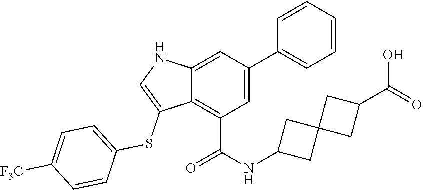 [SMILES: O=C(NC1CC2(C1)CC(C(=O)O)C2)c1cc(-c2ccccc2)cc2[nH]cc(Sc3ccc(C(F)(F)F)cc3)c12]

Starting with phenylboronic acid and Example 112, the title product was obtained as a white solid by following the same procedures as described in Step 4 and Step 5 of Example 75. LCMS (ESI): 551 [M+1]⁺.

¹H NMR (400 MHz, DMSO-d₆) δ 12.09 (s, 1H), 8.21 (d, J=7.1 Hz, 1H), 7.86 (d, J=2.4 Hz, 1H), 7.79 (d, J=1.6 Hz, 1H), 7.71 (d, J=7.4 Hz, 2H), 7.49 (t, J=7.7 Hz, 4H), 7.37 (d, J=7.3 Hz, 1H), 7.28 (d, J=1.6 Hz, 1H), 7.09 (d, J=8.2 Hz, 2H), 4.04-3.96 (m, 1H), 2.91-2.82 (m, 1H), 2.22-2.16 (m, 2H), 2.15-2.07 (m, 1H), 2.06-1.96 (m, 2H), 1.95-1.90 (m, 1H), 1.75-1.71 (m, 1H), 1.69-1.65 (m, 1H).

### EXAMPLE 114

### (±) 6-(6-(3-cyanophenyl)-3-((4-(trifluoromethyl)phenyl)thio)-1H-indole-4-carboxamido)spiro[3.3]heptane-2-carboxylic acid

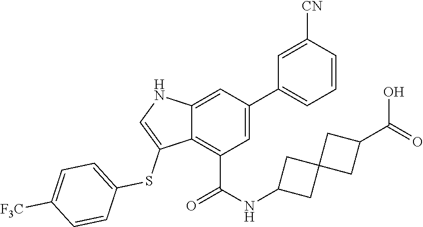 [SMILES: N#Cc1cccc(-c2cc(C(=O)NC3CC4(C3)CC(C(=O)O)C4)c3c(Sc4ccc(C(F)(F)F)cc4)c[nH]c3c2)c1]

Starting with (3-cyanophenyl)boronic acid, the title product was obtained as a white solid by following the same procedures as described in Example 113. LCMS (ESI): 576 [M+1]⁺.

¹H NMR (400 MHz, DMSO-d₆) δ 12.20 (s, 1H), 8.29-8.15 (m, 2H), 8.15-8.02 (m, 1H), 7.96-7.87 (m, 2H), 7.84 (d, J=7.7 Hz, 1H), 7.70 (t, J=7.8 Hz, 1H), 7.51 (d, J=8.3 Hz, 2H), 7.38 (d, J=1.6 Hz, 1H), 7.11 (d, J=8.2 Hz, 2H), 4.08-3.97 (m, 2H), 2.95-2.82 (m, 1H), 2.27-2.10 (m, 3H), 2.09-2.01 (m, 2H), 1.99-1.88 (m, 1H), 1.77-1.62 (m, 1H).

### EXAMPLE 115

(±) 6-(6-(3-carbamoylphenyl)-3-((4-(trifluoromethyl)phenyl)thio)-1H-indole-4-carboxamido)spiro[3.3]heptane-2-carboxylic acid

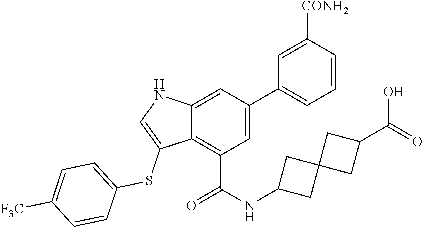 [SMILES: NC(=O)c1cccc(-c2cc(C(=O)NC3CC4(C3)CC(C(=O)O)C4)c3c(Sc4ccc(C(F)(F)F)cc4)c[nH]c3c2)c1]

Starting with (3-carbamoylphenyl)boronic acid, the title product was obtained as a white solid by following the same procedures as described in Example 113. LCMS (ESI): 594 [M+1]⁺.

¹H NMR (400 MHz, DMSO-d₆) δ 12.15 (d, J=2.9 Hz, 1H), 8.31-8.11 (m, 3H), 7.96-7.78 (m, 4H), 7.58 (t, J=7.7 Hz, 1H), 7.52 (d, J=8.4 Hz, 2H), 7.47-7.37 (m, 2H), 7.11 (d, J=8.2 Hz, 2H), 4.04-3.97 (m, 1H), 2.27-2.10 (m, 3H), 2.09-1.90 (m, 3H), 1.82-1.66 (m, 2H).

### EXAMPLE 116

### 4-((5-(3-(methylcarbamoyl)phenyl)-1-(4-(trifluoromethyl)benzyl)-1H-indole-7-carboxamido)methyl)benzoic acid

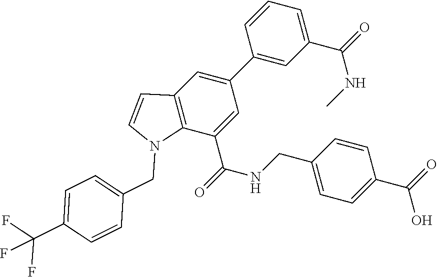 [SMILES: CNC(=O)c1cccc(-c2cc(C(=O)NCc3ccc(C(=O)O)cc3)c3c(ccn3Cc3ccc(C(F)(F)F)cc3)c2)c1]

Starting with (3-(methylcarbamoyl)phenyl)boronic acid, the title product was obtained as a white solid by following the same procedures as described in Example 75. LCMS (ESI): 586.2 [M+1]⁺.

¹H NMR (400 MHz, DMSO-d₆) δ 12.23 (s, 1H), 8.37 (s, 1H), 8.23 (s, 1H), 8.09 (d, J=7.9 Hz, 1H), 7.92-7.82 (m, 3H), 7.72 (d, J=7.9 Hz, 1H), 7.50 (d, J=8.2 Hz, 2H), 7.37 (d, J=1.6 Hz, 1H), 7.06 (d, J=8.2 Hz, 2H), 1.78 (s, 6H), 1.01 (d, J=6.5 Hz, 4H).

### EXAMPLE 117

### 4-((6-(3-cyanophenyl)-3-((4-(trifluoromethyl)phenyl)thio)-1H-indole-4-carboxamido)methyl)benzoic acid

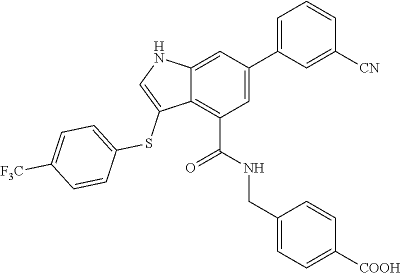 [SMILES: N#Cc1cccc(-c2cc(C(=O)NCc3ccc(C(=O)O)cc3)c3c(Sc4ccc(C(F)(F)F)cc4)c[nH]c3c2)c1]

Starting with the product of Step 2 of Example 112, methyl 4-(aminomethyl)benzoate and (3-cyanophenyl)boronic acid, the title product was obtained as a white solid by following the same procedures as described in Example 75. LCMS (ESI): 572 [M+1]⁺.

¹H NMR (400 MHz, DMSO-d₆) δ 12.23 (d, J=2.8 Hz, 1H), 8.73 (t, J=6.0 Hz, 1H), 8.25 (t, J=1.8 Hz, 1H), 8.13-8.10 (m, 1H), 7.95-7.93 (m, 2H), 7.88-7.80 (m, 3H), 7.71 (t, J=7.8 Hz, 1H), 7.54-7.48 (m, 3H), 7.36 (d, J=8.0 Hz, 2H), 7.12 (d, J=8.3 Hz, 2H), 4.36 (d, J=5.9 Hz, 2H).

### EXAMPLE 118

### 2-(3-(6-(3-cyanophenyl)-3-((4-(trifluoromethyl)phenyl)thio)-1H-indole-4-carboxamido)bicyclo[1.1.1]pentan-1-yl)acetic acid

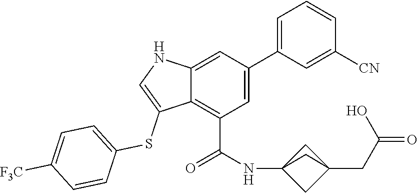 [SMILES: N#Cc1cccc(-c2cc(C(=O)NC34CC(CC(=O)O)(C3)C4)c3c(Sc4ccc(C(F)(F)F)cc4)c[nH]c3c2)c1]

### Step 1 ethyl 2-(3-aminobicyclo[1.1.1]pentan-1-yl)acetate hydrochloride

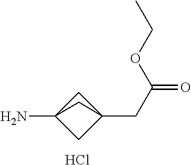

To a solution of ethyl 2-(3-((tert-butoxycarbonyl)amino)bicyclo[1.1.1]pentan-1-yl)acetate (0.70 g) in THF (10 mL) was added 4 M HCl in dioxane (1 mL). After stirring overnight, the mixture was concentrated to give the title compound without further purification to afford as a white solid (0.5 g). LCMS (ESI): 170.1 [M+1]⁺.

### Step 2 2-(3-(6-(3-cyanophenyl)-3-((4-(trifluoromethyl)phenyl)thio)-1H-indole-4-carboxamido)bicyclo[1.1.1]pentan-1-yl)acetic acid

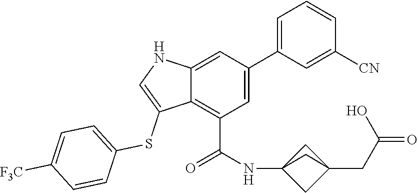 [SMILES: N#Cc1cccc(-c2cc(C(=O)NC34CC(CC(=O)O)(C3)C4)c3c(Sc4ccc(C(F)(F)F)cc4)c[nH]c3c2)c1]

Starting with the product of Step 1, the product of Step 2 of Example 112 and (3-cyanophenyl)boronic acid, the title product was obtained as a white solid by following the same procedures as described in Example 75.

### LCMS (ESI): 562.4 [M+1]⁺.

¹H NMR (400 MHz, DMSO-d₆) δ 12.17 (s, 1H), 8.53 (s, 1H), 8.23 (d, J=1.8 Hz, 1H), 8.16-8.01 (m, 1H), 7.98-7.84 (m, 2H), 7.85-7.75 (m, 1H), 7.69 (t, J=7.8 Hz, 1H), 7.50 (d, J=8.2 Hz, 2H), 7.40 (d, J=1.6 Hz, 1H), 7.10 (d, J=8.3 Hz, 2H), 2.43 (s, 2H), 1.82 (s, 6H).

### EXAMPLE 119

### 3-((6-(3-cyanophenyl)-3-((4-(trifluoromethyl)phenyl)thio)-1H-indole-4-carboxamido)methyl)bicyclo[1.1.1]pentane-1-carboxylic acid

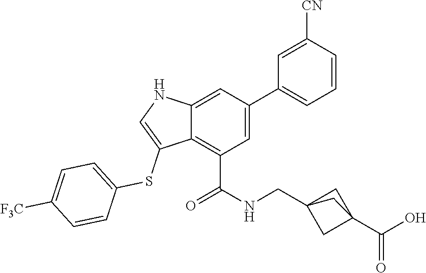 [SMILES: N#Cc1cccc(-c2cc(C(=O)NCC34CC(C(=O)O)(C3)C4)c3c(Sc4ccc(C(F)(F)F)cc4)c[nH]c3c2)c1]

Starting with the product of Step 2 of Example 112, methyl 3-(aminomethyl)bicyclo[1.1.1]pentane-1-carboxylatehydrochloride and (3-cyanophenyl)boronic acid, the title product was obtained as a white solid by following the same procedures as described in Step 4 of Example 75. LCMS (ESI): 562 [M+1]⁺.

¹H NMR (400 MHz, DMSO-d₆) δ 12.21-12.17 (m, 1H), 8.22 (d, J=1.8 Hz, 1H), 8.16-8.06 (m, 2H), 7.93-7.81 (m, 3H), 7.71 (t, J=7.8 Hz, 1H), 7.49 (d, J=8.2 Hz, 2H), 7.41 (d, J=1.6 Hz, 1H), 7.11 (d, J=8.2 Hz, 2H), 3.14 (d, J=5.7 Hz, 2H), 1.79 (s, 6H).

### EXAMPLE 120

### 4-((6-(3-cyanophenyl)-1-methyl-3-((4-(trifluoromethyl)phenyl)thio)-1H-indole-4-carboxamido)methyl)benzoic acid

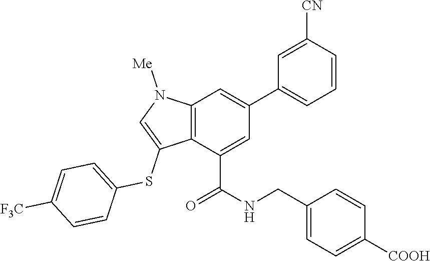 [SMILES: Cn1cc(Sc2ccc(C(F)(F)F)cc2)c2c(C(=O)NCc3ccc(C(=O)O)cc3)cc(-c3cccc(C#N)c3)cc21]

### Step 1 methyl 6-bromo-1-methyl-3-((4-(trifluoromethyl)phenyl)thio)-1H-indole-4-carboxylate

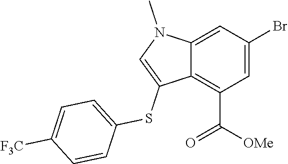 [SMILES: COC(=O)c1cc(Br)cc2c1c(Sc1ccc(C(F)(F)F)cc1)cn2C]

To the product of the step 1 of example 112 (91 mg) in DMF (5 mL) at 0° C. was added NaH (18 mg) was added, then stirred 0.5 h at RT. Then Mel (30 mg) was added to the reaction mixture. After stirring for 2 h, 50 mL water was added. The reaction mixture was extracted 3 times with 50 mL ethyl acetate, washed with brine, dried over anhydrous Na₂SO₄, filtered, concentrated to afford acid (80 mg). LCMS (ESI): 444 [M+1]+

### Step 2 4-((6-(3-cyanophenyl)-1-methyl-3-((4-(trifluoromethyl)phenyl)thio)-1H-indole-4-carboxamido)methyl)benzoic acid

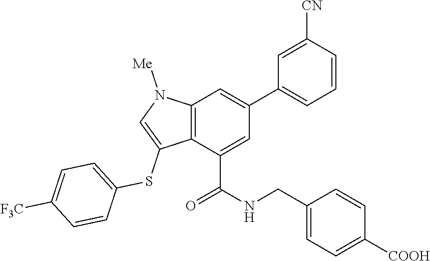 [SMILES: Cn1cc(Sc2ccc(C(F)(F)F)cc2)c2c(C(=O)NCc3ccc(C(=O)O)cc3)cc(-c3cccc(C#N)c3)cc21]

Starting with the product of Step 1, methyl 4-(aminomethyl)benzoate and (3-cyanophenyl)boronic acid, the title product was obtained as a white solid by following the same procedures as described in Example 75. LCMS (ESI): 586.5 [M+1]⁺.

¹H NMR (400 MHz, DMSO-d₆) δ 8.72 (t, J=6.0 Hz, 1H), 8.35 (d, J=1.9 Hz, 1H), 8.23-8.16 (m, 1H), 8.13 (d, J=1.6 Hz, 1H), 7.94 (s, 1H), 7.89-7.79 (m, 3H), 7.73-7.68 (m, 1H), 7.56 (d, J=1.5 Hz, 1H), 7.50 (d, J=8.2 Hz, 2H), 7.35 (d, J=8.0 Hz, 2H), 7.12 (d, J=8.3 Hz, 2H), 4.35 (d, J=5.9 Hz, 2H), 4.00 (s, 3H).

### EXAMPLE 121

### 4-((6-(3-carbamoylphenyl)-1-methyl-3-((4-(trifluoromethyl)phenyl)thio)-1H-indole-4-carboxamido)methyl)benzoic acid

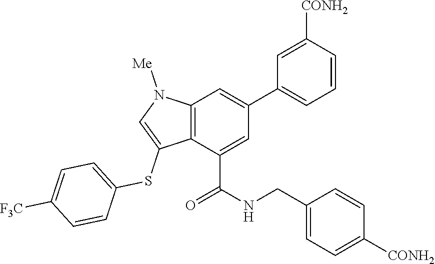 [SMILES: Cn1cc(Sc2ccc(C(F)(F)F)cc2)c2c(C(=O)NCc3ccc(C(N)=O)cc3)cc(-c3cccc(C(N)=O)c3)cc21]

Starting with the product of Step 1 of Example 120, methyl 4-(aminomethyl)benzoate and (3-carbamoylphenyl)boronic acid, the title product was obtained as a white solid by following the same procedures as described Example 75. LCMS (ESI): 604 [M+1]⁺.

¹H NMR (400 MHz, DMSO-d₆) δ 8.73 (t, J=6.0 Hz, 1H), 8.29 (d, J=2.0 Hz, 1H), 8.15 (s, 1H), 8.04 (d, J=1.5 Hz, 1H), 7.95-7.85 (m, 2H), 7.81 (d, J=8.0 Hz, 2H), 7.62-7.54 (m, 3H), 7.52-7.42 (m, 3H), 7.34 (d, J=7.9 Hz, 2H), 7.12 (d, J=8.3 Hz, 2H), 4.34 (d, J=5.9 Hz, 2H), 3.99 (s, 3H).

### EXAMPLE 122

### 4-((5-phenyl-1-(3-(trifluoromethyl)benzyl)-1H-indole-7-carboxamido)methyl)benzoic acid

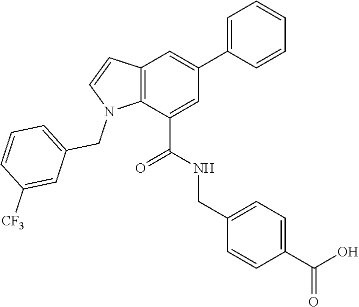 [SMILES: O=C(O)c1ccc(CNC(=O)c2cc(-c3ccccc3)cc3ccn(Cc4cccc(C(F)(F)F)c4)c23)cc1]

Starting with 1-(bromomethyl)-3-(trifluoromethyl)benzene and phenylboronic acid, the title product was obtained as a white solid by following the same procedures as described in Example 75. LCMS (ESI): 529.2 [M+1]⁺.

¹HNMR (400 MHz, DMSO-d₆) δ 12.86 (s, 1H), 9.08 (t, J=6.0 Hz, 1H), 8.03 (d, J=1.8 Hz, 1H), 7.85 (d, J=8.0 Hz, 2H), 7.74 (d, J=7.6 Hz, 2H), 7.64 (d, J=3.2 Hz, 1H), 7.59 (d, J=7.8 Hz, 1H), 7.55 (d, J=1.8 Hz, 1H), 7.51-7.42 (m, 3H), 7.37-7.29 (m, 4H), 7.09 (d, J=7.8 Hz, 1H), 6.74 (d, J=3.2 Hz, 1H), 5.71 (s, 2H), 4.43 (d, J=5.9 Hz, 2H).

### EXAMPLE 123

### 4-((5-(pyridin-3-yl)-1-(3-(trifluoromethyl)benzyl)-1H-indole-7-carboxamido)methyl)benzoic acid

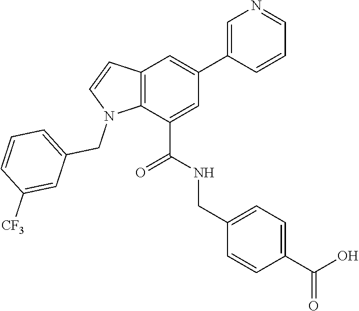 [SMILES: O=C(O)c1ccc(CNC(=O)c2cc(-c3cccnc3)cc3ccn(Cc4cccc(C(F)(F)F)c4)c23)cc1]

Starting with 1-(bromomethyl)-3-(trifluoromethyl)benzene and pyridin-3-ylboronic acid, the title product was obtained as a white solid by following the same procedures as described in Example 75. LCMS (ESI): 530.2 [M+1]⁺.

¹HNMR (400 MHz, DMSO-d₆) δ 12.86 (s, 1H), 9.07 (t, J=6.0 Hz, 1H), 8.98 (d, J=2.4 Hz, 1H), 8.55 (dd, J=4.8, 1.5 Hz, 1H), 8.17-8.09 (m, 2H), 7.85 (d, J=8.1 Hz, 2H), 7.68 (d, J=3.2 Hz, 1H), 7.59 (q, J=3.3 Hz, 2H), 7.53-7.42 (m, 2H), 7.34-7.29 (m, 3H), 7.09 (d, J=7.8 Hz, 1H), 6.76 (d, J=3.2 Hz, 1H), 5.72 (s, 2H), 4.43 (d, J=5.9 Hz, 2H).

### EXAMPLE 124

### 4-((5-(4-fluorophenyl)-1-(3-(trifluoromethyl)benzyl)-1H-indole-7-carboxamido)methyl)benzoic acid

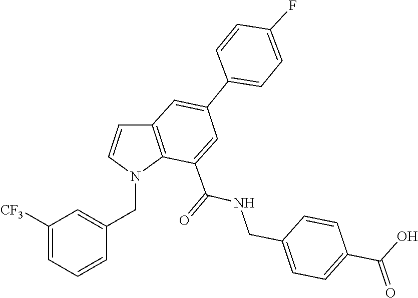 [SMILES: O=C(O)c1ccc(CNC(=O)c2cc(-c3ccc(F)cc3)cc3ccn(Cc4cccc(C(F)(F)F)c4)c23)cc1]

Starting with 1-(bromomethyl)-3-(trifluoromethyl)benzene and (4-fluorophenyl)boronic acid, the title product was obtained as a white solid by following the same procedure described in Example 75. LCMS (ESI): 547.2 [M+1]⁺.

¹H NMR (400 MHz, DMSO-d₆) δ 12.83 (s, 1H), 9.07 (t, J=6.0 Hz, 1H), 8.00 (s, 1H), 7.85 (d, J=7.8 Hz, 2H), 7.77 (dd, J=8.5, 5.4 Hz, 2H), 7.64 (d, J=3.2 Hz, 1H), 7.59 (d, J=7.8 Hz, 1H), 7.52 (s, 1H), 7.45 (t, J=7.8 Hz, 1H), 7.31 (d, J=7.7 Hz, 5H), 7.09 (d, J=7.8 Hz, 1H), 6.73 (d, J=3.1 Hz, 1H), 5.71 (s, 2H), 4.42 (d, J=5.8 Hz, 2H).

### EXAMPLE 125

### 4-((5-(3,5-difluorophenyl)-1-(3-(trifluoromethyl)benzyl)-1H-indole-7-carboxamido)methyl)benzoic acid

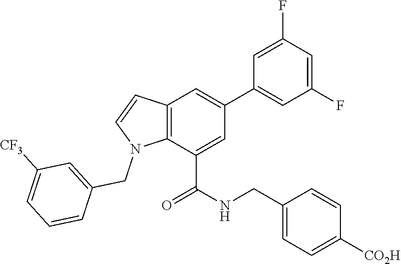 [SMILES: O=C(O)c1ccc(CNC(=O)c2cc(-c3cc(F)cc(F)c3)cc3ccn(Cc4cccc(C(F)(F)F)c4)c23)cc1]

Starting with 1-(bromomethyl)-3-(trifluoromethyl)benzene and (3,5-difluorophenyl)boronic acid, the title product was obtained as a white solid by following the same procedures as described in Example 75.

### LCMS (ESI): 565.5 [M+1]⁺.

¹HNMR (400 MHz, DMSO-d₆) δ 12.87 (s, 1H), 9.06 (t, J=5.9 Hz, 1H), 8.14 (s, 1H), 7.84 (d, J=7.8 Hz, 2H), 7.67 (d, J=3.3 Hz, 1H), 7.63-7.56 (m, 2H), 7.52 (d, J=8.2 Hz, 2H), 7.45 (t, J=7.9 Hz, 1H), 7.35-7.26 (m, 3H), 7.23-7.15 (m, 1H), 7.07 (d, J=7.8 Hz, 1H), 6.75 (d, J=3.2 Hz, 1H), 5.73 (s, 2H), 4.44 (d, J=5.9 Hz, 2H).

### EXAMPLE 126

### 4-((5-(3-cyanophenyl)-1-(3-(trifluoromethyl)benzyl)-1H-indole-7-carboxamido)methyl)benzoic acid

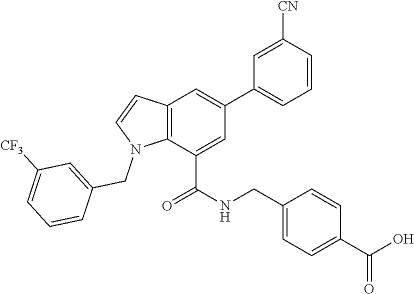 [SMILES: N#Cc1cccc(-c2cc(C(=O)NCc3ccc(C(=O)O)cc3)c3c(ccn3Cc3cccc(C(F)(F)F)c3)c2)c1]

Starting with 1-(bromomethyl)-3-(trifluoromethyl)benzene and (3-cyanophenyl)boronic acid, the title product was obtained as a white solid by following the same procedures as described in Example 75. LCMS (ESI): 554.4 [M+1]⁺.

¹HNMR (400 MHz, DMSO-d₆) δ 12.87 (s, 1H), 9.07 (t, J=5.9 Hz, 1H), 8.23 (d, J=1.9 Hz, 1H), 8.14 (d, J=1.8 Hz, 1H), 8.13-8.08 (m, 1H), 7.84 (d, J=8.1 Hz, 2H), 7.80 (d, J=7.6 Hz, 1H), 7.70-7.65 (m, 2H), 7.63 (d, J=1.9 Hz, 1H), 7.59 (d, J=7.8 Hz, 1H), 7.45 (t, J=7.8 Hz, 1H), 7.33-7.28 (m, 3H), 7.08 (d, J=7.8 Hz, 1H), 6.76 (d, J=3.2 Hz, 1H), 5.73 (s, 2H), 4.44 (d, J=5.9 Hz, 2H).

### EXAMPLE 127

### (S)-4-(1-(5-(3-cyanophenyl)-1-(3-(trifluoromethyl)benzyl)-1H-indole-7-carboxamido)ethyl)benzoic acid

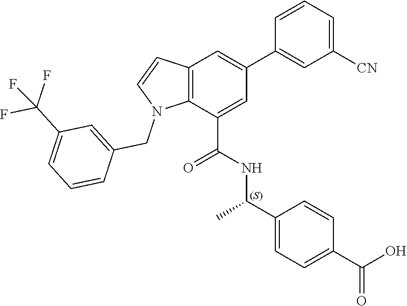

Starting with 1-(bromomethyl)-3-(trifluoromethyl)benzene, (S)-methyl 4-(1-aminoethyl)benzoate and (3-cyanophenyl)boronic acid, the title product was obtained as a white solid by following the same procedure described for Example 75. LCMS (ESI): 568.5[M+1]⁺.

¹HNMR (400 MHz, DMSO-d₆) δ 12.83 (s, 1H), 8.98 (d, J=7.6 Hz, 1H), 8.24 (t, J=1.8 Hz, 1H), 8.15-8.09 (m, 2H), 7.86 (d, J=8.1 Hz, 2H), 7.81 (d, J=7.8 Hz, 1H), 7.71 (t, J=7.8 Hz, 1H), 7.64 (d, J=3.2 Hz, 1H), 7.60-7.55 (m, 2H), 7.44-7.41 (m, 3H), 7.26 (s, 1H), 7.03 (d, J=7.8 Hz, 1H), 6.75 (d, J=3.2 Hz, 1H), 5.71-5.60 (m, 2H), 5.06-5.01 (m, 1H).

### EXAMPLE 128

### (S)-4-(1-(5-(pyridin-3-yl)-1-(3-(trifluoromethyl)benzyl)-1H-indole-7-carboxamido)ethyl)benzoic acid

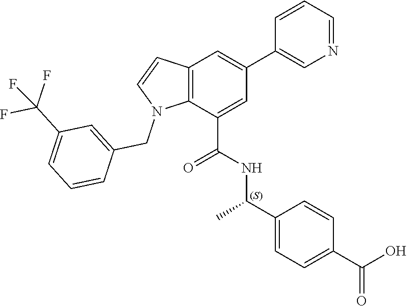

Starting with 1-(bromomethyl)-3-(trifluoromethyl)benzene, (S)-methyl 4-(1-aminoethyl)benzoate and pyridine-3-ylboronic acid, the title product was obtained as a white solid by following the same procedure described for Example 75. LCMS (ESI): 544.5[M+1]⁺.

¹HNMR (400 MHz, DMSO-d₆) δ 9.05 (d, J=2.3 Hz, 1H), 8.99 (d, J=7.6 Hz, 1H), 8.62 (dd, J=4.9, 1.5 Hz, 1H), 8.29 (d, J=8.1, 1H), 8.13 (d, J=1.8 Hz, 1H), 7.87 (d, J=8.1 Hz, 2H), 7.66 (d, J=3.2 Hz, 1H), 7.64-7.55 (m, 3H), 7.44 (d, J=7.9 Hz, 3H), 7.28 (s, 1H), 7.04 (d, J=7.8 Hz, 1H), 6.76 (d, J=3.2 Hz, 1H), 5.75-5.58 (m, 2H), 5.07-5.01 (m, 1H).

### EXAMPLE 129

### (S)-4-(1-(5-phenyl-1-(3-(trifluoromethyl)benzyl)-1H-indole-7-carboxamido)ethyl)benzoic acid

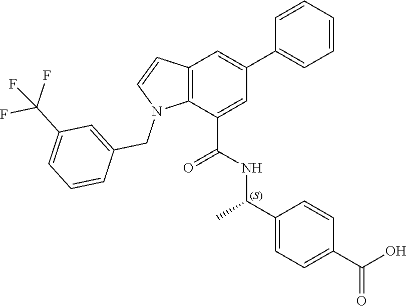

Starting with 1-(bromomethyl)-3-(trifluoromethyl)benzene, (S)-methyl 4-(1-aminoethyl)benzoate and phenylboronic acid (26 mg), the title product was obtained as a white solid by following the same procedure described for Example 75. LCMS (ESI): 543.5[M+1]⁺.

¹HNMR (400 MHz, DMSO-d₆) δ 12.84 (s, 1H), 9.00 (d, J=7.6 Hz, 1H), 8.02 (d, J=1.7 Hz, 1H), 7.87 (d, J=8.1 Hz, 2H), 7.78-7.72 (m, 2H), 7.61 (d, J=3.2 Hz, 1H), 7.58 (d, J=7.8 Hz, 1H), 7.53-7.47 (m, 3H), 7.44-7.41 (m, 3H), 7.35 (t, J=7.4 Hz, 1H), 7.27 (s, 1H), 7.04 (d, J=7.8 Hz, 1H), 6.73 (d, J=3.1 Hz, 1H), 5.72-5.57 (m, 2H), 5.07-5.01 (m, J=7.1 Hz, 1H).

### EXAMPLE 130

### (S)-4-(1-(5-(3,5-difluorophenyl)-1-(3-(trifluoromethyl)benzyl)-1H-indole-7-carboxamido)ethyl)benzoic acid

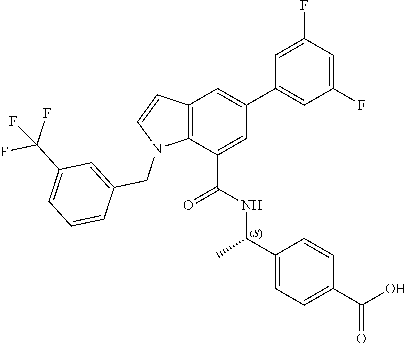

Starting with 1-(bromomethyl)-3-(trifluoromethyl)benzene, (S)-methyl 4-(1-aminoethyl)benzoate and (3,5-difluorophenyl)boronic acid, the title product was obtained as a white solid by following the same procedure described for Example 75. LCMS (ESI): 579.5[M+1]⁺.

¹HNMR (400 MHz, DMSO-d₆) δ 12.83 (s, 1H), 8.96 (d, J=7.6 Hz, 1H), 8.13 (d, J=1.8 Hz, 1H), 7.89-7.84 (m, 2H), 7.64 (d, J=3.2 Hz, 1H), 7.58 (d, J=7.9 Hz, 1H), 7.55-7.48 (m, 3H), 7.44-7.40 (m, 3H), 7.25 (s, 1H), 7.22-7.18 (m, 1H), 7.03 (d, J=7.8 Hz, 1H), 6.74 (d, J=3.2 Hz, 1H), 5.70-5.60 (m, 2H), 5.07-5.01 (m, J=7.1 Hz, 1H).

### EXAMPLE 131

### 4-(1-(5-phenyl-1-(3-(trifluoromethyl)benzyl)-1H-indole-7-carboxamido)cyclopropyl)benzoic acid

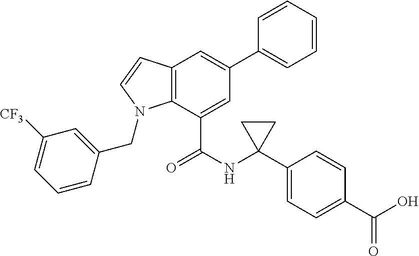 [SMILES: O=C(O)c1ccc(C2(NC(=O)c3cc(-c4ccccc4)cc4ccn(Cc5cccc(C(F)(F)F)c5)c34)CC2)cc1]

Starting with 1-(bromomethyl)-3-(trifluoromethyl)benzene, methyl 4-(1-aminocyclopropyl)benzoate and phenylboronic acid, the title product was obtained as a white solid by following the same procedures as described in Example 75. LCMS (ESI): 555.4 [M+1]⁺.

¹H NMR (400 MHz, DMSO) δ 12.69 (s, 1H), 9.34 (s, 1H), 8.06 (d, J=2.0 Hz, 1H), 7.80 (t, J=8.1 Hz, 4H), 7.68 (d, J=2.0 Hz, 1H), 7.61 (d, J=7.9 Hz, 1H), 7.56-7.43 (m, 4H), 7.37 (t, J=7.4 Hz, 1H), 7.33 7.21 (m, 3H), 7.02 (d, J=7.8 Hz, 1H), 6.75 (d, J=3.2 Hz, 1H), 5.73 (s, 2H), 1.24 (q, J=5.1 Hz, 2H), 1.02 (q, J=5.1 Hz, 2H).

### EXAMPLE 132

### 4-(1-(5-(3,5-difluorophenyl)-1-(3-(trifluoromethyl)benzyl)-1H-indole-7-carboxamido)cyclopropyl)benzoic acid

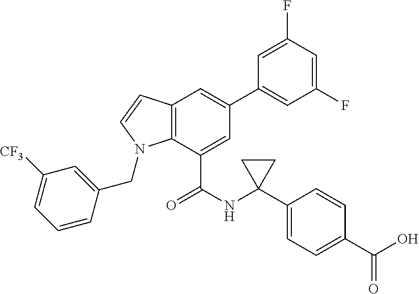 [SMILES: O=C(O)c1ccc(C2(NC(=O)c3cc(-c4cc(F)cc(F)c4)cc4ccn(Cc5cccc(C(F)(F)F)c5)c34)CC2)cc1]

Starting with 1-(bromomethyl)-3-(trifluoromethyl)benzene, methyl 4-(1-aminocyclopropyl)benzoate and (3,5-difluorophenyl)boronic acid, the title product was obtained as a white solid by following the same procedures as described in Example 75. LCMS (ESI): 591.2 [M+1]⁺.

¹H NMR (400 MHz, DMSO-d₆) δ 12.70 (s, 1H), 9.32 (s, 1H), 8.17 (d, J=1.8 Hz, 1H), 7.79 (d, J=8.1 Hz, 2H), 7.73 (d, J=1.9 Hz, 1H), 7.66-7.52 (m, 4H), 7.48 (t, J=7.8 Hz, 1H), 7.27 (d, J=7.8 Hz, 3H), 7.24 7.12 (m, 1H), 7.01 (d, J=7.8 Hz, 1H), 6.76 (d, J=3.1 Hz, 1H), 5.74 (s, 2H), 1.29-1.13 (m, 2H), 1.13-0.95 (m, 2H).

### EXAMPLE 133

### 4-(1-(5-(3-cyanophenyl)-1-(3-(trifluoromethyl)benzyl)-1H-indole-7-carboxamido)cyclopropyl)benzoic acid

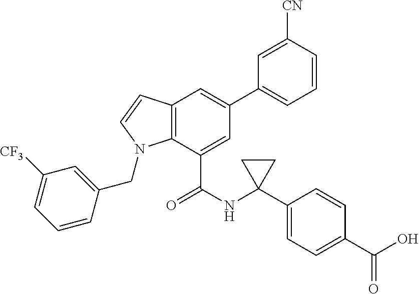 [SMILES: N#Cc1cccc(-c2cc(C(=O)NC3(c4ccc(C(=O)O)cc4)CC3)c3c(ccn3Cc3cccc(C(F)(F)F)c3)c2)c1]

Starting with 1-(bromomethyl)-3-(trifluoromethyl)benzene, methyl 4-(1-aminocyclopropyl)benzoate and (3-cyanophenyl)boronic acid, the title product was obtained as a white solid by following the same procedures as described in Example 75. LCMS (ESI): 580.6 [M+1]⁺.

¹H NMR (400 MHz, DMSO-d₆) δ 12.76 (s, 1H), 9.36 (t, J=2.6 Hz, 1H), 8.31 (s, 1H), 8.22-8.14 (m, 2H), 7.83 (d, J=7.7 Hz, 1H), 7.81-7.68 (m, 4H), 7.62 (d, J=7.9 Hz, 1H), 7.56 (d, J=3.2 Hz, 1H), 7.49 (t, J=7.8 Hz, 1H), 7.34-7.22 (m, 3H), 7.00 (d, J=7.8 Hz, 1H), 6.76 (d, J=3.1 Hz, 1H), 5.75 (s, 2H), 1.28-1.24 (m, 2H), 1.08-0.97 (m, 2H).

### EXAMPLE 134

### (S)-4-(1-(5-(4-fluorophenyl)-1-(3-(trifluoromethyl)benzyl)-1H-indazole-7-carboxamido)ethyl)benzoic acid

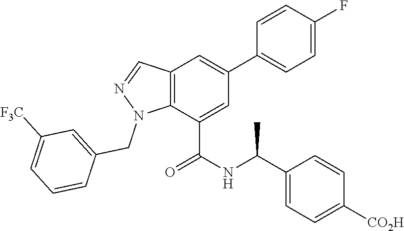 [SMILES: C[C@H](NC(=O)c1cc(-c2ccc(F)cc2)cc2cnn(Cc3cccc(C(F)(F)F)c3)c12)c1ccc(C(=O)O)cc1]

### Step 1 methyl 5-bromo-1-(3-(trifluoromethyl)benzyl)-1H-indazole-7-carboxylate

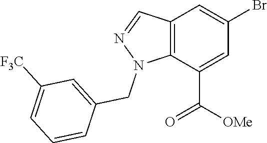 [SMILES: COC(=O)c1cc(Br)cc2cnn(Cc3cccc(C(F)(F)F)c3)c12]

To a mixture of methyl 5-bromo-1H-indazole-7-carboxylate (500 mg) and Cs₂CO₃ (769 mg) in anhydrous DMF (10 mL) at 0° C. was added 1-(bromomethyl)-3-(trifluoromethyl)benzene (564 mg). The reaction mixture was then warmed to rt and stirred for 1 h. The reaction mixture was poured into 100 mL ice-water, extracted 3×50 mL EtOAc. The combined organic layer was washed with 50 mL brine, dried over anhydrous Na₂SO₄ and concentrated. The residue was purified by silica gel column chromatography (petroleum ether/ethyl acetate: from 100:0 to 10:1) to afford the title compound as a white solid (530 mg).

### LCMS (ESI): 413.2 [M+1]⁺.

### Step 2 5-bromo-1-(3-(trifluoromethyl)benzyl)-1H-indazole-7-carboxylic acid

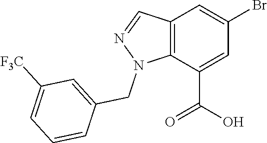 [SMILES: O=C(O)c1cc(Br)cc2cnn(Cc3cccc(C(F)(F)F)c3)c12]

To a solution of the product of Step 1 (500 mg) in MeOH/H₂O (10 mL/2.0 mL) was added LiOH—H₂O (254 mg) at rt. The reaction mixture was stirred at 60° C. for 1 h. After being cooled to 0° C., the reaction mixture was treated with 6 N HCl to adjusted pH≈6. The reaction mixture was extracted 3×30 mL EtOAc. The combined organic layer washed with 20 mL brine, dried over anhydrous Na₂SO₄, concentrated.

The residue was purified by silica gel column chromatography (DCM/MeOH: from 100:0 to 10:1) to afford the title compound as a white solid (460 mg). LCMS (ESI): 399.3 [M+1]⁺.

### Step 3 methyl (S)-4-(1-(5-bromo-1-(3-(trifluoromethyl)benzyl)-1H-indazole-7-carboxamido)ethyl)benzoate

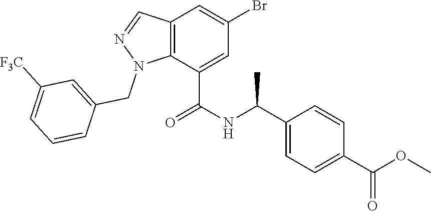 [SMILES: COC(=O)c1ccc([C@H](C)NC(=O)c2cc(Br)cc3cnn(Cc4cccc(C(F)(F)F)c4)c23)cc1]

To a mixture of the product of Step 2 (460 mg), methyl (S)-4-(1-aminoethyl)benzoate (233 mg) and HATU (456 mg) in anhydrous DMF (100 mL) at 0° C. was added DIPEA (0.52 mL). After stirring at rt overnight, the reaction mixture was quenched with 100 mL water and extracted 3×100 mL EtOAc. The combined organic layer was washed with 100 mL brine, dried over anhydrous Na₂SO₄ and concentrated. The residue was purified by silica gel column chromatography (petroleum ether/ethyl acetate: from 100:0 to 5:1) to afford the title compound as a white solid (0.6 g). LCMS (ESI): 560.2 [M+1]⁺.

### Step 4 methyl (S)-4-(1-(5-(4-fluorophenyl)-1-(3-(trifluoromethyl)benzyl)-1H-indazole-7-carboxamido)ethyl)benzoate

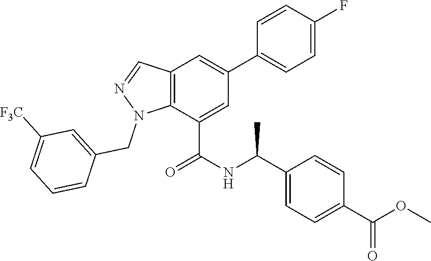 [SMILES: COC(=O)c1ccc([C@H](C)NC(=O)c2cc(-c3ccc(F)cc3)cc3cnn(Cc4cccc(C(F)(F)F)c4)c23)cc1]

A mixture of the product of Step 3 (30 mg), (4-fluorophenyl)boronic acid (15 mg), Pd(dppf)₂Cl₂ (4 mg, 0.01 mmol), AcOK (16 mg) in 1,4-dioxane/H₂O (5 ml/0.5 mL) was stirred for 1 h at 100° C. under nitrogen. The reaction mixture was concentrated and purified by silica gel column chromatography (PE/EA=5:1) to afford the title compound as a yellow solid (27 mg). LCMS (ESI): 576.3 [M+1]⁺.

### Step 5 (S)-4-(1-(5-(4-fluorophenyl)-1-(3-(trifluoromethyl)benzyl)-1H-indazole-7-carboxamido)ethyl)benzoic acid

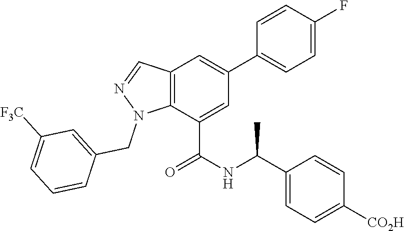 [SMILES: C[C@H](NC(=O)c1cc(-c2ccc(F)cc2)cc2cnn(Cc3cccc(C(F)(F)F)c3)c12)c1ccc(C(=O)O)cc1]

To a solution of the product of Step 4 (27 mg) in MeOH/H₂O (5 mL/0.5 mL) was added LiOH—H₂O (16 mg). The reaction mixture was stirred at 60° C. for 1 h. After being cooled to 0° C., the mixture was treated with 6 N HCl to pH≈6. The reaction mixture was then extracted 3×20 mL EtOAc. The combined organic layer was washed with 20 mL brine, dried over anhydrous Na₂SO₄ and concentrated. The residue was purified by silica gel column chromatography (DCM/MeOH=10:1) to afford the title compound as a white solid (23 mg). LCMS (ESI): 562 [M+1]⁺.

¹HNMR (400 MHz, DMSO-d₆) δ 12.80 (s, 1H), 9.24 (d, J=7.6 Hz, 1H), 8.36 (s, 1H), 8.22 (s, 1H), 7.91-7.77 (m, 5H), 7.59 (d, J=7.8 Hz, 1H), 7.45 (dd, J=22.7, 7.9 Hz, 3H), 7.35 (dd, J=16.3, 7.6 Hz, 3H), 7.12 (d, J=7.8 Hz, 1H), 5.85 (s, 2H), 5.15 (p, J=7.1 Hz, 1H), 1.37 (d, J=7.1 Hz, 3H).

### EXAMPLE 135

### (S)-4-(1-(5-phenyl-1-(3-(trifluoromethyl)benzyl)-1H-indazole-7-carboxamido)ethyl)benzoic acid

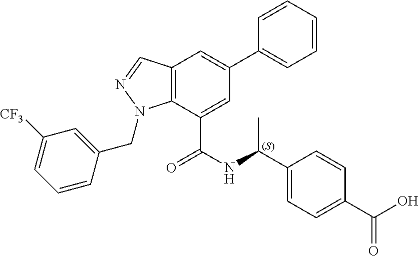

Starting with phenylboronic acid, the title product was obtained as a white solid by following the same procedures as described in Example 134. LCMS (ES+): 544.5 [M+1]⁺.

¹H NMR (400 MHz, DMSO-d₆) 12.85 (s, 1H), 9.33 (d, J=7.6 Hz, 1H), 8.36 (s, 1H), 8.23 (d, J=1.7 Hz, 1H), 7.92-7.82 (m, 3H), 7.80 (d, J=7.6 Hz, 2H), 7.59 (d, J=7.8 Hz, 1H), 7.57-7.45 (m, 4H), 7.45-7.36 (m, 2H), 7.34 (s, 1H), 7.12 (d, J=7.8 Hz, 1H), 5.84 (s, 2H), 5.20-5.10 (m, 1H), 1.37 (d, J=7.0 Hz, 3H).

### EXAMPLE 136

### (S)-4-(1-(5-(3,5-difluorophenyl)-1-(3-(trifluoromethyl)benzyl)-1H-indazole-7-carboxamido)ethyl)benzoic acid

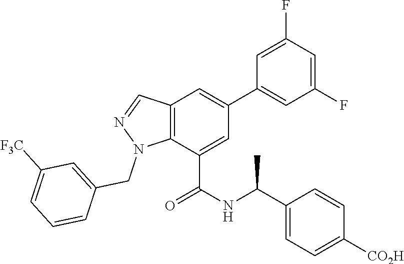 [SMILES: C[C@H](NC(=O)c1cc(-c2cc(F)cc(F)c2)cc2cnn(Cc3cccc(C(F)(F)F)c3)c12)c1ccc(C(=O)O)cc1]

Starting with (3,5-difluorophenyl)boronic acid, the title product was obtained as a white solid by following the same procedures described in Example 134. LCMS (ESI): 580.5 [M+1]⁺.

¹HNMR (400 MHz, DMSO-d₆) δ 12.84 (s, 1H), 9.21 (d, J=7.6 Hz, 1H), 8.38 (s, 1H), 8.35 (d, J=1.7 Hz, 1H), 7.87 (d, J=8.0 Hz, 3H), 7.63-7.54 (m, 3H), 7.51-7.40 (m, 3H), 7.32 (s, 1H), 7.27 (tt, J=9.3, 2.3 Hz, 1H), 7.11 (d, J=7.8 Hz, 1H), 5.85 (s, 2H), 5.14 (p, J=7.1 Hz, 1H), 1.39 (d, J=7.1 Hz, 3H).

### EXAMPLE 137

### 4-((5-phenyl-1-(3-(trifluoromethyl)benzyl)-1H-indazole-7-carboxamido)methyl)benzoic acid

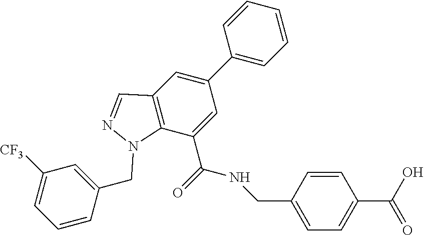 [SMILES: O=C(O)c1ccc(CNC(=O)c2cc(-c3ccccc3)cc3cnn(Cc4cccc(C(F)(F)F)c4)c23)cc1]

Starting with methyl 4-(aminomethyl)benzoate and phenylboronic acid, the title product was obtained as a white solid by following the same procedure described in Example 134. LCMS (ESI): 530.2 [M+1]⁺.

¹HNMR (400 MHz, DMSO-d₆) δ 12.92 (s, 1H), 9.35 (t, J=6.0 Hz, 1H), 8.37 (s, 1H), 8.25 (d, J=1.7 Hz, 1H), 7.90-7.83 (m, 3H), 7.79 (d, J=7.6 Hz, 2H), 7.61 (d, J=7.8 Hz, 1H), 7.52-7.48 (m, 3H), 7.42-7.36 (m, 3H), 7.36 (s, 1H), 7.18 (d, J=7.8 Hz, 1H), 5.93 (s, 2H), 4.53 (d, J=5.8 Hz, 2H).

### EXAMPLE 138

### 4-((5-(4-fluorophenyl)-1-(3-(trifluoromethyl)benzyl)-1H-indazole-7-carboxamido)methyl)benzoic acid

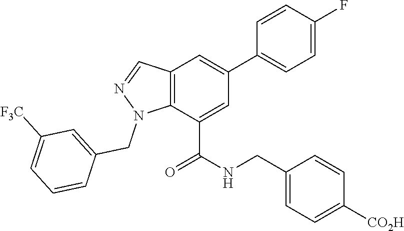 [SMILES: O=C(O)c1ccc(CNC(=O)c2cc(-c3ccc(F)cc3)cc3cnn(Cc4cccc(C(F)(F)F)c4)c23)cc1]

Starting with methyl 4-(aminomethyl)benzoate, the title product was obtained as a white solid by following the same procedures described in Example 134. LCMS (ESI): 548.5 [M+1]⁺.

¹H NMR (400 MHz, DMSO-d₆) δ 12.80 (s, 1H), 9.24 (d, J=7.6 Hz, 1H), 8.36 (s, 1H), 8.22 (s, 1H), 7.91-7.77 (m, 5H), 7.59 (d, J=7.8 Hz, 1H), 7.50-7.40 (m, 3H), 7.39-7.30 (m, 3H), 7.12 (d, J=7.8 Hz, 1H), 5.85 (s, 2H), 5.15 (p, J=7.1 Hz, 1H), 1.37 (d, J=7.1 Hz, 3H).

### EXAMPLE 139

### 4-((5-(3,5-difluorophenyl)-1-(3-(trifluoromethyl)benzyl)-1H-indazole-7-carboxamido)methyl)benzoic acid

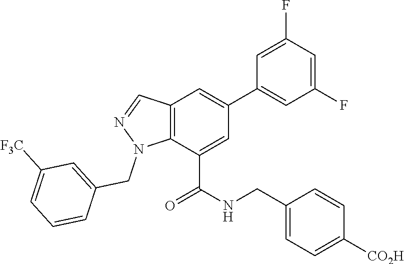 [SMILES: O=C(O)c1ccc(CNC(=O)c2cc(-c3cc(F)cc(F)c3)cc3cnn(Cc4cccc(C(F)(F)F)c4)c23)cc1]

Starting with (3,5-difluorophenyl)boronic acid and methyl 4-(aminomethyl)benzoate, the title product was obtained as a white solid by following the same procedures described in Example 134. LCMS (ESI): 566.4 [M+1]⁺.

¹H NMR (400 MHz, DMSO-d₆) δ 12.84 (s, 1H), 9.21 (d, J=7.6 Hz, 1H), 8.38 (s, 1H), 8.35 (d, J=1.7 Hz, 1H), 7.87 (d, J=8.0 Hz, 3H), 7.63-7.54 (m, 3H), 7.51-7.40 (m, 3H), 7.32 (s, 1H), 7.27 (tt, J=9.3, 2.3 Hz, 1H), 7.11 (d, J=7.8 Hz, 1H), 5.85 (s, 2H), 5.14 (p, J=7.1 Hz, 1H), 1.39 (d, J=7.1 Hz, 3H).

### EXAMPLE 140

### 4-(1-(5-phenyl-1-(3-(trifluoromethyl)benzyl)-1H-indazole-7-carboxamido)cyclopropyl)benzoic acid

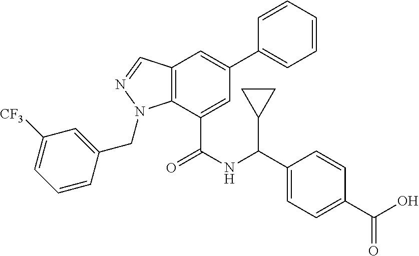 [SMILES: O=C(O)c1ccc(C(NC(=O)c2cc(-c3ccccc3)cc3cnn(Cc4cccc(C(F)(F)F)c4)c23)C2CC2)cc1]

Starting with methyl 4-(1-aminocyclopropyl)benzoate and phenylboronic acid, the title product was obtained as a white solid by following the same procedures as described in Example 134. LCMS (ESI): 556 [M+1]⁺.

¹H NMR (400 MHz, DMSO-d₆) δ 12.75 (s, 1H), 9.59 (s, 1H), 8.37 (s, 1H), 8.28 (d, J=1.7 Hz, 1H), 7.85 (d, J=7.6 Hz, 2H), 7.81-7.75 (m, 2H), 7.63 (d, J=7.8 Hz, 1H), 7.57-7.46 (m, 3H), 7.42 (t, J=7.4 Hz, 1H), 7.35 (s, 1H), 7.31-7.26 (m, 2H), 7.10 (d, J=7.9 Hz, 1H), 5.93 (s, 2H), 1.33-1.28 (m, 2H), 1.20-1.14 (m, 2H).

### EXAMPLE 141

### 4-((5-phenyl-1-(quinolin-2-ylmethyl)-1H-indole-7-carboxamido)methyl)benzoic acid

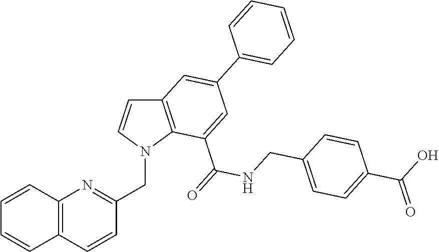 [SMILES: O=C(O)c1ccc(CNC(=O)c2cc(-c3ccccc3)cc3ccn(Cc4ccc5ccccc5n4)c23)cc1]

Starting with 2-(bromomethyl)quinoline and phenylboronic acid, the title product was obtained as a white solid (21 mg) by following the same procedure described for Example 75. LCMS (ESI): 512.5[M+1]⁺.

¹HNMR (400 MHz, DMSO-d₆) δ 8.90 (t, J=5.8 Hz, 1H), 8.19 (d, J=8.5 Hz, 1H), 8.02 (d, J=1.8 Hz, 1H), 7.96-7.89 (m, 2H), 7.75-7.73 (m, 3H), 7.69-7.62 (m, 3H), 7.57 (t, J=7.5 Hz, 1H), 7.50 (d, J=1.8 Hz, 1H), 7.47 (t, J=7.7 Hz, 2H), 7.33 (t, J=7.4 Hz, 1H), 7.06 (d, J=7.9 Hz, 2H), 6.79-6.73 (m, 2H), 5.90 (s, 2H), 4.29 (d, J=5.7 Hz, 2H).

### EXAMPLE 142

### 4-((5-(4-fluorophenyl)-1-(quinolin-2-ylmethyl)-1H-indole-7-carboxamido)methyl)benzoic acid

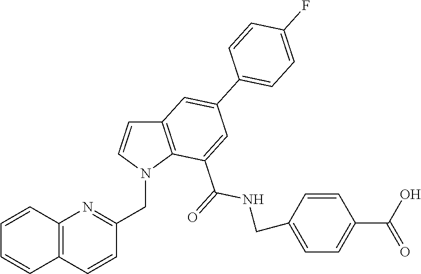 [SMILES: O=C(O)c1ccc(CNC(=O)c2cc(-c3ccc(F)cc3)cc3ccn(Cc4ccc5ccccc5n4)c23)cc1]

Starting with 2-(bromomethyl)quinoline and (4-fluorophenyl)boronic acid, the title product was obtained as a white solid by following the same procedures as described in Example 75. LCMS (ESI): 530.2 [M+1]⁺.

¹HNMR (400 MHz, DMSO-d₆) δ 12.77 (s, 1H), 8.96 (t, J=5.9 Hz, 1H), 8.19 (d, J=8.5 Hz, 1H), 8.01 (d, J=1.8 Hz, 1H), 7.94-7.88 (m, 2H), 7.81-7.69 (m, 3H), 7.66-7.59 (m, 3H), 7.57 (t, J=7.5 Hz, 1H), 7.51 (d, J=1.8 Hz, 1H), 7.30 (t, J=8.8 Hz, 2H), 7.13 (d, J=8.0 Hz, 2H), 6.75 (d, J=3.1 Hz, 1H), 6.71 (d, J=8.6 Hz, 1H), 5.91 (s, 2H), 4.33 (d, J=5.8 Hz, 2H).

### EXAMPLE 143

### (S)-4-(1-(5-phenyl-1-(quinolin-2-ylmethyl)-1H-indole-7-carboxamido)ethyl)benzoic acid

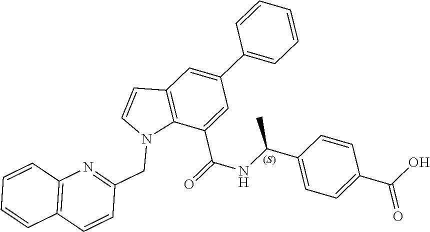

Starting with 2-(bromomethyl)quinoline, methyl (S)-4-(1-aminoethyl)benzoate and phenylboronic acid, the title product was obtained as a white solid (10 mg) by following the same procedures as described in Example 75. LCMS (ESI): 526.2 [M+1]⁺.

¹HNMR (400 MHz, DMSO-d₆) δ 12.79 (s, 1H), 8.86 (d, J=7.6 Hz, 1H), 8.16 (d, J=8.6 Hz, 1H), 8.02 (s, 1H), 7.91 (dd, J=11.7, 8.4 Hz, 2H), 7.74 (t, J=8.6 Hz, 5H), 7.62 (d, J=3.2 Hz, 1H), 7.56 (t, J=7.5 Hz, 1H), 7.48 (dd, J=15.1, 7.5 Hz, 3H), 7.36 (d, J=7.5 Hz, 1H), 7.32 (d, J=8.2 Hz, 2H), 6.75 (d, J=3.1 Hz, 1H), 6.68 (d, J=8.6 Hz, 1H), 5.90-5.78 (m, 2H), 4.97 (p, J=7.1 Hz, 1H), 1.02 (d, J=6.9 Hz, 3H).

### EXAMPLE 144

(S)-4-(1-(5-(4-fluorophenyl)-1-(quinolin-2-ylmethyl)-1H-indole-7-carboxamido)ethyl)benzoic acid

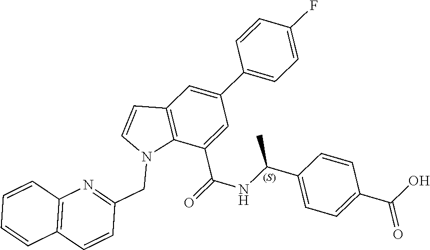

Starting with 2-(bromomethyl)quinoline, methyl (S)-4-(1-aminoethyl)benzoate and (4-fluorophenyl)boronic acid, the title product was obtained as a white solid by following the same procedures as described in Example 75. LCMS (ESI): 544.2 [M+1]⁺.

¹HNMR (400 MHz, DMSO-d₆) δ 12.73 (s, 1H), 8.84 (d, J=7.6 Hz, 1H), 8.16 (d, J=8.5 Hz, 1H), 8.00 (s, 1H), 7.90 (t, J=9.4 Hz, 2H), 7.78 (dd, J=8.4, 5.5 Hz, 2H), 7.73 (d, J=8.0 Hz, 3H), 7.63 (d, J=3.1 Hz, 1H), 7.56 (t, J=7.5 Hz, 1H), 7.43 (s, 1H), 7.32 (dd, J=8.4, 5.6 Hz, 4H), 6.74 (d, J=3.1 Hz, 1H), 6.68 (d, J=8.6 Hz, 1H), 5.84 (d, J=5.4 Hz, 2H), 4.96 (t, J=7.2 Hz, 1H), 1.02 (d, J=7.1 Hz, 3H).

### EXAMPLE 145

### ((1-([1,1′-biphenyl]-3-ylmethyl)-5-phenyl-1H-indole-7-carboxamido)methyl)benzoic acid

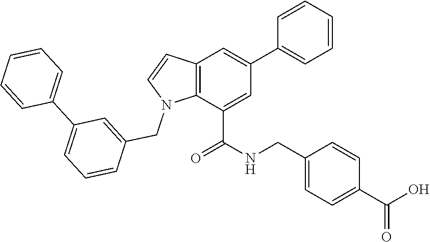 [SMILES: O=C(O)c1ccc(CNC(=O)c2cc(-c3ccccc3)cc3ccn(Cc4cccc(-c5ccccc5)c4)c23)cc1]

Starting with 3-(bromomethyl)-1,1′-biphenyl and phenylboronic acid, the title product was obtained as a white solid by following the same procedures as described in Example 75. LCMS (ESI): 537.3 [M+1]⁺.

¹HNMR (400 MHz, DMSO-d₆) δ 9.12-9.05 (m, 1H), 7.99 (s, 1H), 7.86-7.79 (m, 2H), 7.72 (d, J=7.7 Hz, 2H), 7.65 (d, J=3.2 Hz, 1H), 7.55-7.49 (m, 4H), 7.48-7.40 (m, 4H), 7.36-7.24 (m, 6H), 6.88 (d, J=7.7 Hz, 1H), 6.71 (d, J=3.1 Hz, 1H), 5.68 (s, 2H), 4.46-4.38 (m, 2H).

### EXAMPLE 146

### 4-((1-(3-methoxybenzyl)-5-phenyl-1H-indole-7-carboxamido)methyl)benzoic acid

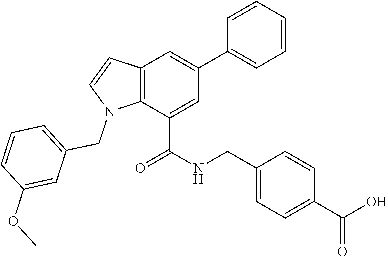 [SMILES: COc1cccc(Cn2ccc3cc(-c4ccccc4)cc(C(=O)NCc4ccc(C(=O)O)cc4)c32)c1]

Starting with 1-(bromomethyl)-3-methoxybenzene and phenylboronic acid, the title product was obtained as a white solid by following the same procedures as described in Example 75. LCMS (ESI): 491.4 [M+1]⁺.

¹HNMR (400 MHz, DMSO-d₆) δ 12.87 (s, 1H), 9.07 (t, J=6.0 Hz, 1H), 8.00 (s, 1H), 7.85 (d, J=8.1 Hz, 2H), 7.74 (d, J=7.7 Hz, 2H), 7.58 (d, J=3.2 Hz, 1H), 7.55 (s, 1H), 7.47 (t, J=7.6 Hz, 2H), 7.34 (d, J=7.4 Hz, 1H), 7.31 (d, J=8.2 Hz, 2H), 7.13 (t, J=7.9 Hz, 1H), 6.81-6.77 (m, 1H), 6.70 (d, J=3.2 Hz, 1H), 6.47-6.44 (m, 1H), 6.42 (d, J=7.6 Hz, 1H), 5.59 (s, 2H), 4.49 (d, J=5.9 Hz, 2H), 3.65 (s, 3H).

### EXAMPLE 147

### 4-((1-([1,1′-biphenyl]-4-ylmethyl)-5-phenyl-1H-indole-7-carboxamido)methyl)benzoic acid

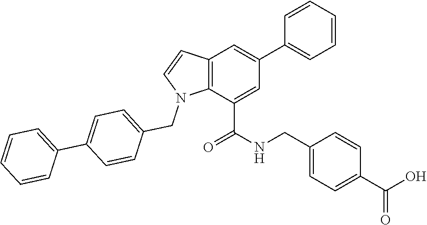 [SMILES: O=C(O)c1ccc(CNC(=O)c2cc(-c3ccccc3)cc3ccn(Cc4ccc(-c5ccccc5)cc4)c23)cc1]

Starting with 4-(bromomethyl)-1,1′-biphenyl and phenylboronic acid, the title product was obtained as a white solid by following the same procedures as described in Example 75. LCMS (ESI): 537.5 [M+1]⁺.

¹HNMR (400 MHz, DMSO-d₆) δ 9.07 (t, J=6.0 Hz, 1H), 8.01 (d, J=1.7 Hz, 1H), 7.85 (d, J=7.9 Hz, 2H), 7.74 (d, J=7.6 Hz, 2H), 7.65 (d, J=3.2 Hz, 1H), 7.59 (d, J=7.6 Hz, 2H), 7.55 (s, 1H), 7.51-7.40 (m, 6H), 7.40-7.30 (m, 4H), 6.96 (d, J=8.0 Hz, 2H), 6.72 (d, J=3.1 Hz, 1H), 5.65 (s, 2H), 4.51 (d, J=5.9 Hz, 2H).

### EXAMPLE 148

### 4-((1-([1,1′-biphenyl]-4-ylmethyl)-5-(4-fluorophenyl)-1H-indole-7-carboxamido)methyl)benzoic acid

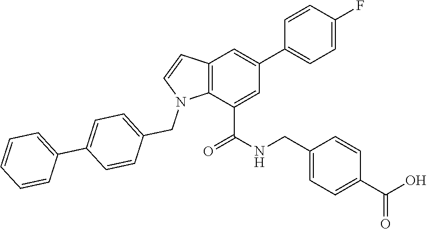 [SMILES: O=C(O)c1ccc(CNC(=O)c2cc(-c3ccc(F)cc3)cc3ccn(Cc4ccc(-c5ccccc5)cc4)c23)cc1]

Starting with 4-(bromomethyl)-1,1′-biphenyl and (4-fluorophenyl)boronic acid, the title product was obtained as a white solid by following the same procedures as described in Example 75. LCMS (ESI): 555.3 [M+1]⁺.

¹HNMR Spectrum: (400 MHz, DMSO-d₆) δ 12.85 (s, 1H), 9.05 (t, J=6.0 Hz, 1H), 7.98 (s, 1H), 7.84 (d, J=7.9 Hz, 2H), 7.78-7.72 (m, 2H), 7.64 (d, J=3.2 Hz, 1H), 7.57 (d, J=7.6 Hz, 2H), 7.51 (s, 1H), 7.46 (d, J=8.0 Hz, 2H), 7.42 (t, J=7.6 Hz, 2H), 7.38-7.31 (m, 3H), 7.29 (t, J=8.7 Hz, 2H), 6.94 (d, J=7.9 Hz, 2H), 6.70 (d, J=3.1 Hz, 1H), 5.64 (s, 2H), 4.50 (d, J=5.9 Hz, 2H).

### EXAMPLE 149

### 4-((1-([1,1′-biphenyl]-4-ylmethyl)-5-(3,5-difluorophenyl)-1H-indole-7-carboxamido)methyl)benzoic acid

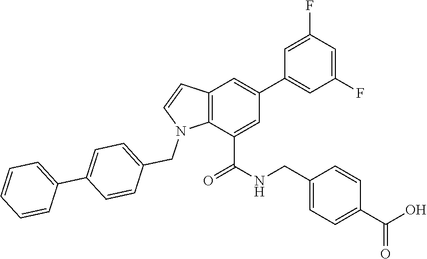 [SMILES: O=C(O)c1ccc(CNC(=O)c2cc(-c3cc(F)cc(F)c3)cc3ccn(Cc4ccc(-c5ccccc5)cc4)c23)cc1]

Starting with 4-(bromomethyl)-1,1′-biphenyl and (3,5-difluorophenyl)boronic acid, the title product was obtained as a white solid by following the same procedures as described in Example 75. LCMS (ESI): 573.4 [M+1]⁺.

¹HNMR (400 MHz, DMSO-d₆) δ 12.86 (s, 1H), 9.04 (t, J=6.0 Hz, 1H), 8.12 (s, 1H), 7.84 (d, J=8.0 Hz, 2H), 7.68 (d, J=3.2 Hz, 1H), 7.61 (s, 1H), 7.58 (d, J=7.7 Hz, 2H), 7.53-7.49 (m, 2H), 7.48-7.41 (m, 4H), 7.39-7.32 (m, 3H), 7.22-7.14 (m, 1H), 6.93 (d, J=8.0 Hz, 2H), 6.73 (d, J=3.1 Hz, 1H), 5.67 (s, 2H), 4.53 (d, J=5.9 Hz, 2H).

### EXAMPLE 150

### (S)-4-(1-(1-([1,1′-biphenyl]-4-ylmethyl)-5-phenyl-1H-indole-7-carboxamido)ethyl)benzoic acid

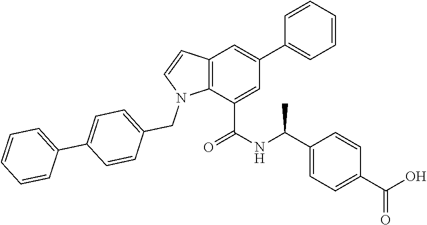 [SMILES: C[C@H](NC(=O)c1cc(-c2ccccc2)cc2ccn(Cc3ccc(-c4ccccc4)cc3)c12)c1ccc(C(=O)O)cc1]

Starting with 4-(bromomethyl)-1,1′-biphenyl, methyl (S)-4-(1-aminoethyl)benzoate and phenylboronic acid, the title product was obtained as a white solid by following the same procedures as described in Example 75. LCMS (ESI): 551.4 [M+1]⁺.

¹HNMR (400 MHz, DMSO-d₆) δ 12.84 (s, 1H), 8.98 (d, J=7.8 Hz, 1H), 7.98 (s, 1H), 7.89 (d, J=8.0 Hz, 2H), 7.73 (d, J=7.7 Hz, 2H), 7.63 (d, J=3.2 Hz, 1H), 7.54 (d, J=7.7 Hz, 2H), 7.52-7.45 (m, 5H), 7.44 7.39 (m, 4H), 7.36-7.29 (m, 2H), 6.93 (d, J=8.0 Hz, 2H), 6.70 (d, J=3.1 Hz, 1H), 5.58 (s, 2H), 5.22 5.14 (m, 1H), 1.32 (d, J=7.0 Hz, 3H).

### EXAMPLE 151

### (S)-4-(1-(1-([1,1′-biphenyl]-4-ylmethyl)-5-(4-fluorophenyl)-1H-indole-7-carboxamido)ethyl)benzoic acid

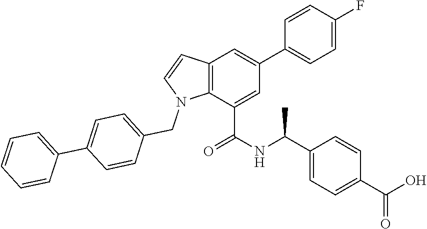 [SMILES: C[C@H](NC(=O)c1cc(-c2ccc(F)cc2)cc2ccn(Cc3ccc(-c4ccccc4)cc3)c12)c1ccc(C(=O)O)cc1]

Starting with 4-(bromomethyl)-1,1′-biphenyl, methyl (S)-4-(1-aminoethyl)benzoate and (4-fluorophenyl) boronic acid, the title product was obtained as a white solid by following the same procedures as described in Example 75. LCMS (ESI): 569.5 [M+1]⁺.

¹HNMR (400 MHz, DMSO-d₆) δ 12.85 (s, 1H), 8.97 (d, J=7.8 Hz, 1H), 7.97 (d, J=1.8 Hz, 1H), 7.89 (d, J=8.1 Hz, 2H), 7.79-7.73 (m, 2H), 7.63 (d, J=3.2 Hz, 1H), 7.54 (d, J=7.7 Hz, 2H), 7.50 (d, J=8.1 Hz, 2H), 7.46 (d, J=1.9 Hz, 1H), 7.44-7.39 (m, 4H), 7.35-7.27 (m, 3H), 6.92 (d, J=8.0 Hz, 2H), 6.69 (d, J=3.1 Hz, 1H), 5.58 (s, 2H), 5.22-5.14 (m, 1H), 1.32 (d, J=7.0 Hz, 3H).

### EXAMPLE 152

### (S)-4-(1-(1-([1,1′-biphenyl]-4-ylmethyl)-5-(3,5-difluorophenyl)-1H-indole-7-carboxamido)ethyl)benzoic acid

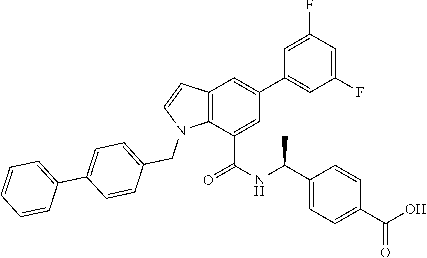 [SMILES: C[C@H](NC(=O)c1cc(-c2cc(F)cc(F)c2)cc2ccn(Cc3ccc(-c4ccccc4)cc3)c12)c1ccc(C(=O)O)cc1]

Starting with 4-(bromomethyl)-1,1′-biphenyl, methyl (S)-4-(1-aminoethyl)benzoate and (3,5-difluorophenyl)boronic acid, the title product was obtained as a white solid by following the same procedures as described in Example 75. LCMS (ESI): 587.5 [M+1]⁺.

¹HNMR (400 MHz, DMSO-d₆) δ 8.93 (d, J=7.8 Hz, 1H), 8.09 (d, J=1.9 Hz, 1H), 7.88 (d, J=8.0 Hz, 2H), 7.66 (d, J=3.2 Hz, 1H), 7.57-7.47 (m, 7H), 7.45-7.37 (m, 4H), 7.36-7.29 (m, 1H), 7.22-7.13 (m, 1H), 6.91 (d, J=8.0 Hz, 2H), 6.71 (d, J=3.1 Hz, 1H), 5.58 (s, 2H), 5.22-5.14 (m, 1H), 1.34 (d, J=7.0 Hz, 3H).

### EXAMPLE 153

### 4-((4-(phenylamino)-1-(quinolin-2-ylmethyl)-1H-indole-7-carboxamido)methyl)benzoic acid

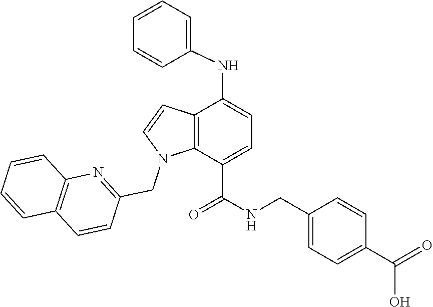 [SMILES: O=C(O)c1ccc(CNC(=O)c2ccc(Nc3ccccc3)c3ccn(Cc4ccc5ccccc5n4)c23)cc1]

Starting with 2-(bromomethyl)quinolone, methyl 4-(aminomethyl)benzoate and aniline, the title product was obtained as a white solid following the same procedure described in Example 64. LCMS (ESI): 527.2 [M+1]⁺.

¹HNMR (400 MHz, DMSO-d₆) δ 8.66 (t, J=6.1 Hz, 1H), 8.27 (s, 1H), 8.16 (d, J=8.5 Hz, 1H), 7.91 (dd, J=15.3, 8.3 Hz, 2H), 7.72 (t, J=7.7 Hz, 1H), 7.60 (d, J=7.9 Hz, 2H), 7.55 (t, J=7.5 Hz, 1H), 7.44 (d, J=3.3 Hz, 1H), 7.30 (t, J=7.7 Hz, 2H), 7.24 (d, J=7.9 Hz, 2H), 7.18 (d, J=8.0 Hz, 1H), 7.13 (d, J=8.0 Hz, 2H), 6.95-6.87 (m, 2H), 6.84 (d, J=8.0 Hz, 1H), 6.65 (d, J=8.5 Hz, 1H), 5.89 (s, 2H), 4.30 (d, J=5.9 Hz, 2H).

### EXAMPLE 154

### 4-((1-([1,1′-biphenyl]-4-ylmethyl)-4-(phenylamino)-1H-indole-7-carboxamido)methyl) benzoic acid

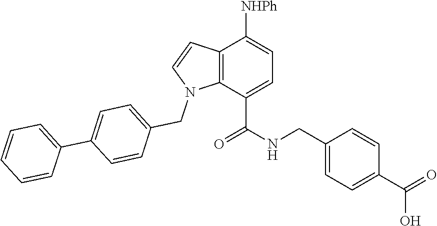

Starting with 4-(bromomethyl)-1,1′-biphenyl, methyl 4-(aminomethyl)benzoate and aniline, the title product was obtained as a white solid by following the same procedure described in Example 64. LCMS (ESI): 552.2 [M+1]⁺.

### EXAMPLE 155

### 4-((1-(3-methoxybenzyl)-4-(phenylamino)-1H-indole-7-carboxamido)methyl)benzoic acid

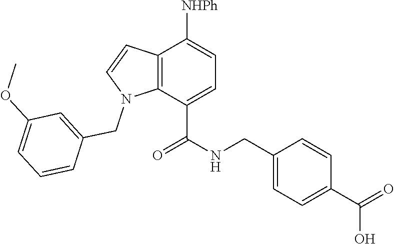

Starting with 1-(bromomethyl)-3-methoxybenzene, methyl 4-(aminomethyl)benzoate and aniline, the title product was obtained as a white solid by following the same procedure described in Example 64.

### LCMS (ESI): 506.2 [M+1]⁺.

¹HNMR (400 MHz, DMSO-d₆) δ 8.77 (t, J=6.0 Hz, 1H), 8.24 (s, 1H), 7.83 (d, J=8.1 Hz, 2H), 7.40 (d, J=3.2 Hz, 1H), 7.31-7.26 (m, 4H), 7.26-7.16 (m, 3H), 7.12 (t, J=7.9 Hz, 1H), 6.91 (t, J=7.2 Hz, 1H), 6.86-6.80 (m, 2H), 6.80-6.76 (m, 1H), 6.50-6.37 (m, 2H), 5.59 (s, 2H), 4.44 (d, J=6.0 Hz, 2H), 3.65 (s, 3H).

### EXAMPLE 156

### 4-((1-(naphthalen-2-ylmethyl)-4-(phenylamino)-1H-indole-7-carboxamido)methyl)benzoic acid

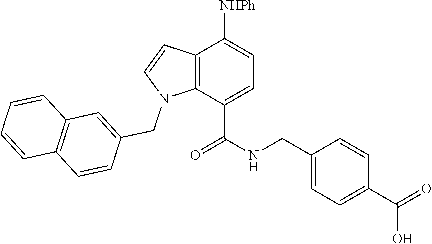

Starting with 2-(bromomethyl)naphthalene, methyl 4-(aminomethyl)benzoate and aniline, the title product was obtained as a white solid by following the same procedure described in Example 64. LCMS (ESI): 622.1 [M+1]⁺.

¹HNMR (400 MHz, DMSO-d₆) δ 8.71-8.66 (m, 1H), 8.25 (s, 1H), 7.89-7.81 (m, 1H), 7.78-7.70 (m, 4H), 7.51 (d, J=3.3 Hz, 1H), 7.49-7.44 (m, 2H), 7.41 (s, 1H), 7.34-7.26 (m, 4H), 7.23 (d, J=7.9 Hz, 2H), 7.16 (d, J=8.0 Hz, 1H), 7.10-7.06 (m, 1H), 6.96-6.85 (m, 2H), 6.81 (d, J=8.0 Hz, 1H), 5.77 (s, 2H), 4.45-4.41 (m, 2H).

### EXAMPLE 157

### 4-((5-(3,5-difluorophenyl)-1-(naphthalen-2-ylmethyl)-1H-indazole-7-carboxamido)methyl)benzoic acid

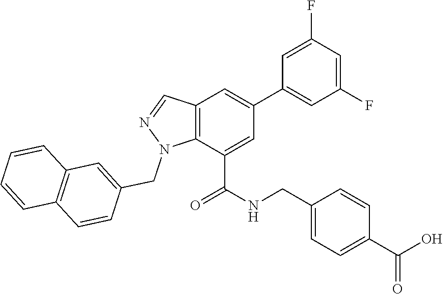 [SMILES: O=C(O)c1ccc(CNC(=O)c2cc(-c3cc(F)cc(F)c3)cc3cnn(Cc4ccc5ccccc5c4)c23)cc1]

Starting with 2-(bromomethyl)naphthalene methyl 4-(aminomethyl)benzoate and (3,5-difluorophenyl) boronic acid, the title product was obtained as a white solid by following the same procedures described in Example 134. LCMS (ESI): 548.3 [M+1]⁺.

¹H NMR (400 MHz, DMSO-d₆) δ 12.81 (s, 1H), 9.17 (t, J=5.9 Hz, 1H), 8.39 (s, 1H), 8.36 (d, J=1.7 Hz, 1H), 7.92-7.82 (m, 2H), 7.77-7.74 (m, 3H), 7.60-7.53 (m, 2H), 7.51-7.41 (m, 3H), 7.30-7.21 (m, 4H), 7.11 (dd, J=8.5, 1.7 Hz, 1H), 6.04 (s, 2H), 4.55 (d, J=5.7 Hz, 2H).

### EXAMPLE 158

### (S)-4-(1-(1-(3-methoxybenzyl)-4-(phenylamino)-1H-indole-7-carboxamido)ethyl)benzoic acid

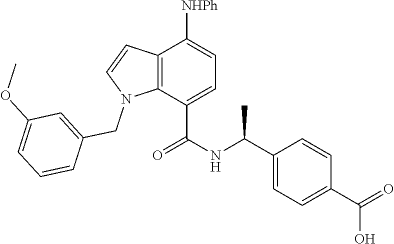

Starting with 1-(bromomethyl)-3-methoxybenzene (100 mg), methyl (S)-4-(1-aminoethyl)benzoate (100 mg) and aniline (70 mg), the title product was obtained as a white solid (25 mg) by following the same procedure described in Example 64. LCMS (ESI): 622.1 [M+1]⁺.

¹HNMR (400 MHz, DMSO-d₆) δ 12.79 (s, 1H), 8.69 (d, J=7.9 Hz, 1H), 8.22 (s, 1H), 7.86 (d, J=8.1 Hz, 2H), 7.52-7.37 (m, 3H), 7.32-7.26 (m, 2H), 7.25-7.20 (m, 2H), 7.16 (d, J=8.0 Hz, 1H), 7.09 (t, J=7.9 Hz, 1H), 6.90 (t, J=7.2 Hz, 1H), 6.86-6.81 (m, 2H), 6.79-6.73 (m, 1H), 6.44-6.41 (m, 1H), 6.39 (d, J=7.6 Hz, 1H), 5.60-5.54 (m, 1H), 5.50-5.44 (m, 1H), 5.15-5.07 (m, 1H), 3.65 (s, 3H), 1.31 (d, J=7.1 Hz, 3H).

### EXAMPLE 159

### 4-((1-(naphthalen-2-ylmethyl)-5-phenyl-1H-indole-7-carboxamido)methyl)benzoic acid

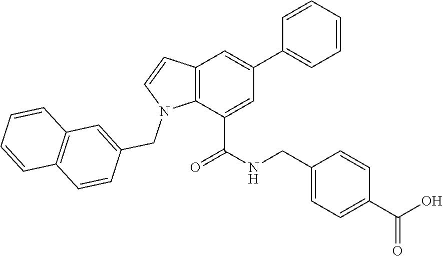 [SMILES: O=C(O)c1ccc(CNC(=O)c2cc(-c3ccccc3)cc3ccn(Cc4ccc5ccccc5c4)c23)cc1]

Starting with 2-(bromomethyl)naphthalene and phenylboronic acid, the title product was obtained as a white solid by following the same procedures as described in Example 75. LCMS (ESI): 511.4 [M+1]⁺.

¹HNMR (400 MHz, DMSO-d₆) δ 8.98 (t, J=5.9 Hz, 1H), 8.02 (s, 1H), 7.89-7.82 (m, 1H), 7.79-7.70 (m, 6H), 7.69 (d, J=3.2 Hz, 1H), 7.51 (s, 1H), 7.49-7.43 (m, 4H), 7.41 (s, 1H), 7.33 (t, J=7.4 Hz, 1H), 7.24 (d, J=7.9 Hz, 2H), 7.10 (d, J=8.5 Hz, 1H), 6.74 (d, J=3.1 Hz, 1H), 5.79 (s, 2H), 4.45 (d, J=5.8 Hz, 2H).

### EXAMPLE 160

### 4-((5-(4-fluorophenyl)-1-(naphthalen-2-ylmethyl)-1H-indole-7-carboxamido)methyl)benzoic acid

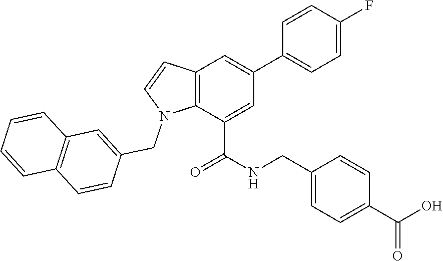 [SMILES: O=C(O)c1ccc(CNC(=O)c2cc(-c3ccc(F)cc3)cc3ccn(Cc4ccc5ccccc5c4)c23)cc1]

Starting with 2-(bromomethyl)naphthalene and (4-fluorophenyl)boronic acid, the title product was obtained as a white solid by following the same procedures as described in Example 75. LCMS (ESI): 529.5[M+1]⁺.

¹HNMR (400 MHz, DMSO-d₆) δ 12.79 (s, 1H), 8.95 (t, J=5.9 Hz, 1H), 7.98 (s, 1H), 7.86-7.81 (m, 1H), 7.77-7.70 (m, 6H), 7.69 (d, J=3.1 Hz, 1H), 7.49-7.43 (m, 3H), 7.39 (s, 1H), 7.27 (t, J=8.7 Hz, 2H), 7.22 (d, J=7.9 Hz, 2H), 7.08 (d, J=8.5 Hz, 1H), 6.72 (d, J=3.2 Hz, 1H), 5.78 (s, 2H), 4.44 (d, J=5.8 Hz, 2H).

### EXAMPLE 161

### 4-((5-(3,5-difluorophenyl)-1-(naphthalen-2-ylmethyl)-1H-indole-7-carboxamido)methyl)benzoic acid

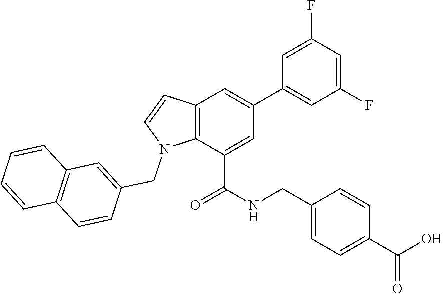 [SMILES: O=C(O)c1ccc(CNC(=O)c2cc(-c3cc(F)cc(F)c3)cc3ccn(Cc4ccc5ccccc5c4)c23)cc1]

Starting with 2-(bromomethyl)naphthalene and (3,5-difluorophenyl)boronic acid (50 mg), the title product was obtained as a white solid by following the same procedures as described in Example 75. LCMS (ESI): 547.4[M+1]⁺.

¹HNMR (400 MHz, DMSO-d₆) δ 12.81 (s, 1H), 8.92 (t, J=5.9 Hz, 1H), 8.12 (d, J=1.9 Hz, 1H), 7.86-7.81 (m, 1H), 7.75 (d, J=8.5 Hz, 1H), 7.73-7.69 (m, 4H), 7.55 (d, J=1.9 Hz, 1H), 7.50-7.43 (m, 4H), 7.37 (s, 1H), 7.20 (d, J=7.9 Hz, 2H), 7.18-7.12 (m, 1H), 7.08-7.04 (m, 1H), 6.74 (d, J=3.2 Hz, 1H), 5.80 (s, 2H), 4.45 (d, J=5.8 Hz, 2H).

### EXAMPLE 162

### (S)-4-(1-(1-(naphthalen-2-ylmethyl)-5-phenyl-1H-indole-7-carboxamido)ethyl)benzoic acid

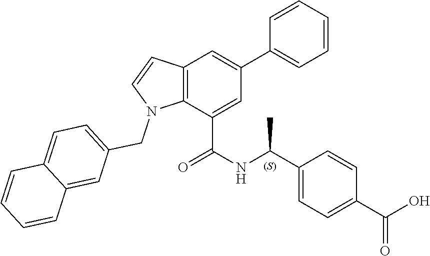

Starting with 2-(bromomethyl)naphthalene, methyl (S)-4-(1-aminoethyl)benzoate and phenylboronic acid, the title product was obtained as a white solid by following the same procedures as described in Example 75. LCMS (ESI): 525.2 [M+1]⁺.

¹H NMR (400 MHz, DMSO-d₆) δ 12.80 (s, 1H), 8.89 (d, J=7.5 Hz, 1H), 8.01 (d, J=1.8 Hz, 1H), 7.82 (t, J=6.9 Hz, 3H), 7.74 (t, J=7.1 Hz, 4H), 7.67 (d, J=3.2 Hz, 1H), 7.52-7.31 (m, 9H), 7.06 (d, J=8.5 Hz, 1H), 6.73 (d, J=3.2 Hz, 1H), 5.84-5.63 (m, 2H), 5.11 (p, J=7.2 Hz, 1H), 1.12 (d, J=7.0 Hz, 3H).

### EXAMPLE 163

(S)-4-(1-(5-(4-fluorophenyl)-1-(naphthalen-2-ylmethyl)-1H-indole-7-carboxamido)ethyl)benzoic acid

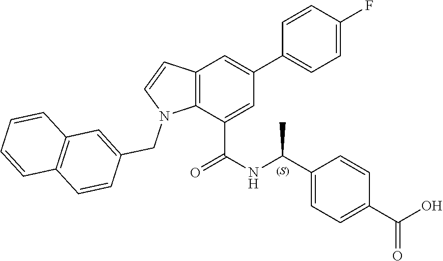

Starting with 2-(bromomethyl)naphthalene, methyl (S)-4-(1-aminoethyl)benzoate and (4-fluorophenyl) boronic acid, the title product was obtained as a white solid by following the same procedure described for Example 75. LCMS (ESI): 543.5[M+1]⁺.

¹HNMR (400 MHz, DMSO-d₆) δ 8.87 (d, J=7.6 Hz, 1H), 7.98 (d, J=1.8 Hz, 1H), 7.84-7.82 (m, J=7.5 Hz, 3H), 7.77-7.1 (m, 4H), 7.68 (d, J=3.2 Hz, 1H), 7.47-7.44 (m, 6H), 7.31 (t, J=8.7 Hz, 2H), 7.05 (d, J=8.5, 1H), 6.72 (d, J=3.1 Hz, 1H), 5.81-5.62 (m, 2H), 5.12-5.07 (m, 1H).

### EXAMPLE 164

### (S)-4-(1-(5-(3,5-difluorophenyl)-1-(naphthalen-2-ylmethyl)-1H-indole-7-carboxamido)ethyl)benzoic acid

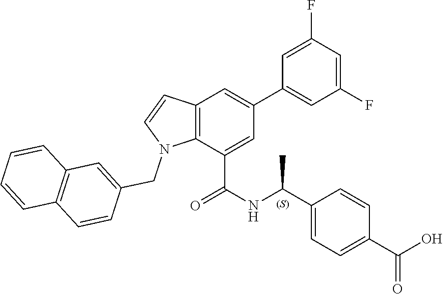

Starting with 2-(bromomethyl)naphthalene, methyl (S)-4-(1-aminoethyl)benzoate and (3,5-difluorophenyl) boronic acid, the title product was obtained as a white solid by following the same procedure described for Example 75. LCMS (ESI): 561.5[M+1]⁺.

¹HNMR (400 MHz, DMSO-d₆) δ 12.81 (s, 1H), 8.83 (d, J=7.6 Hz, 1H), 8.11 (d, J=1.8 Hz, 1H), 7.84-7.79 (m, 3H), 7.76-7.69 (m, 3H), 7.53-7.43 (m, 5H), 7.40 (d, J=8.1 Hz, 2H), 7.37 (s, 1H), 7.20-7.16 (m, 1H), 7.04 (d, J=8.5,1H), 6.74 (d, J=3.2 Hz, 1H), 5.80-5.65 (m, 2H), 5.12-5.06 (m, 1H), 1.13 (d, J=7.0 Hz, 3H).

### EXAMPLE 165

### 4-((1-(naphthalen-2-ylmethyl)-5-phenyl-1H-indazole-7-carboxamido)methyl)benzoic acid

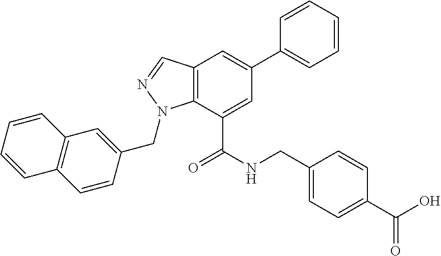 [SMILES: O=C(O)c1ccc(CNC(=O)c2cc(-c3ccccc3)cc3cnn(Cc4ccc5ccccc5c4)c23)cc1]

Starting with 2-(bromomethyl)naphthalene, methyl 4-(aminomethyl)benzoate and phenylboronic acid, the title product was obtained as a white solid by following the same procedures described in Example 134.

### LCMS (ESI): 512.4 [M+1]⁺.

¹H NMR (400 MHz, DMSO-d₆) δ 12.81 (s, 1H), 9.21 (t, J=5.9 Hz, 1H), 8.37 (s, 1H), 8.23 (d, J=1.7 Hz, 1H), 7.87-7.81 (m, 2H), 7.78-7.44 (m, 10H), 7.38 (t, J=7.4 Hz, 1H), 7.30 (d, J=8.0 Hz, 3H), 7.15-7.10 (m, 1H), 6.03 (s, 2H), 4.55 (d, J=5.8 Hz, 2H).

### EXAMPLE 166

### 4-((5-(4-fluorophenyl)-1-(naphthalen-2-ylmethyl)-1H-indazole-7-carboxamido)methyl)benzoic acid

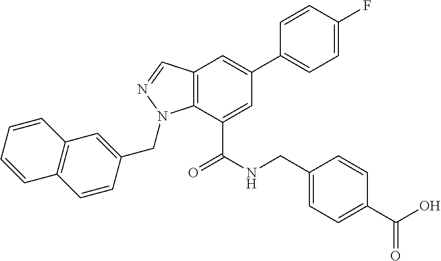 [SMILES: O=C(O)c1ccc(CNC(=O)c2cc(-c3ccc(F)cc3)cc3cnn(Cc4ccc5ccccc5c4)c23)cc1]

Starting with 2-(bromomethyl)naphthalene, methyl 4-(aminomethyl)benzoate and (4-fluorophenyl)boronic acid, the title product was obtained as a white solid by following the same procedures described in Example 134. LCMS (ESI): 530.6 [M+1]⁺.

¹H NMR (400 MHz, DMSO-d₆) δ 12.82 (s, 1H), 9.20 (t, J=5.9 Hz, 1H), 8.37 (s, 1H), 8.22 (d, J=1.7 Hz, 1H), 7.87-7.71 (m, 8H), 7.51-7.42 (m, 3H), 7.37-7.19 (m, 5H), 7.15-7.09 (m, 1H), 6.03 (s, 2H), 4.54 (d, J=5.8 Hz, 2H).

### EXAMPLE 167

### (S)-4-(1-(1-(naphthalen-2-ylmethyl)-5-phenyl-1H-indazole-7-carboxamido)ethyl)benzoic acid

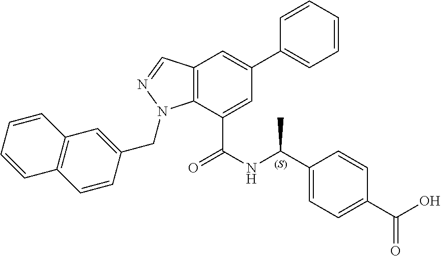

### Step 1 methyl 5-bromo-1-(naphthalen-2-ylmethyl)-1H-indazole-7-carboxylate

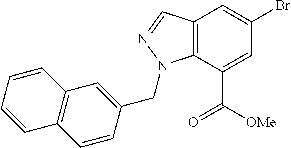 [SMILES: COC(=O)c1cc(Br)cc2cnn(Cc3ccc4ccccc4c3)c12]

To a mixture of methyl 5-bromo-1H-indazole-7-carboxylate (500 mg) and Cs₂CO₃ (769 mg) in anhydrous DMF (10 mL) at 0° C. was added 2-(bromomethyl)naphthalene (564 mg). The reaction mixture was then warmed to rt and stirred overnight. The reaction mixture was poured into 100 mL ice-water, extracted by 3×50 mL ethyl acetate. The combined organic layer was washed with 50 mL brine, dried over anhydrous Na₂SO₄ and concentrated. The residue was purified by silica gel column chromatography (petroleum ether/ethyl acetate: from 100:0 to 10:1) to afford Isomer 1 as the title compound (500 mg, fast eluting) and Isomer 2 (a regioisomer) as white solids.

Isomer 1 (the title compound): LCMS (ES+): 395.1 [M+1]⁺. ¹HNMR (400 MHz, DMSO-d₆) δ 8.41-8.34 (m, 2H), 7.88-7.76 (m, 4H), 7.48 (dt, J=6.4, 3.4 Hz, 2H), 7.43 (s, 1H), 7.02 (dd, J=8.5, 1.8 Hz, 1H), 6.02 (s, 2H), 3.79 (s, 3H).

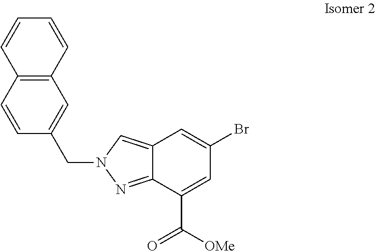 [SMILES: COC(=O)c1cc(Br)cc2cn(Cc3ccc4ccccc4c3)nc12]

Isomer 2: LCMS (ES+): 395.1 [M+1]⁺. HNMR (400 MHz, DMSO-d₆) δ 8.70 (s, 1H), 8.34 (d, J=1.9 Hz, 1H), 7.91 (ddd, J=14.8, 8.9, 5.5 Hz, 6H), 7.57-7.50 (m, 2H), 7.47 (dd, J=8.5, 1.8 Hz, 1H), 5.92 (s, 2H), 3.89 (s, 3H).

### Step 2 5-bromo-1-(naphthalen-2-ylmethyl)-1H-indazole-7-carboxylic acid

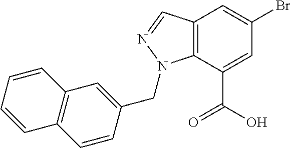 [SMILES: O=C(O)c1cc(Br)cc2cnn(Cc3ccc4ccccc4c3)c12]

To a solution of the product of Step 1 (500 mg) in MeOH/H₂O (10 mL/2 mL) was added LiOH—H₂O (254 mg) at rt. The reaction mixture was stirred at 60° C. for 1 h. After being cooled to 0° C., the reaction mixture was treated with 6 N HCl to adjusted pH≈6. The reaction mixture was extracted 3×30 mL EtOAc. The combined organic layer was washed with 20 mL brine, dried over anhydrous Na₂SO₄, and concentrated. The residue was purified by silica gel column chromatography (DCM/MeOH: from 100:0 to 10:1) to afford the title compound as a white solid (479 mg). LCMS (ESI): 381.1 [M+1]⁺.

### Step 3 methyl (S)-4-(1-(5-bromo-1-(naphthalen-2-ylmethyl)-1H-indazole-7-carboxamido)ethyl)benzoate

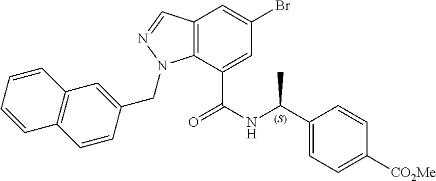

To a mixture of the product of Step 2 (398 mg), methyl (S)-4-(1-aminoethyl)benzoate (215 mg) and HATU (456 mg) in anhydrous DMF (10 mL) at 0° C. was added DIPEA (0.52 mL). After stirring at rt overnight, the reaction mixture was quenched with 10 mL water and extracted 3×100 mL EtOAc. The combined organic layer was washed with 10 mL brine, dried over anhydrous Na₂SO₄ and concentrated. The residue was purified by silica gel column chromatography (petroleum ether/ethyl acetate: from 100:0 to 5:1) to afford the title compound as a white solid (0.5 g). LCMS (ESI): 542.1 [M+1]⁺.

### Step 4 methyl (S)-4-(1-(1-(naphthalen-2-ylmethyl)-5-phenyl-1H-indazole-7-carboxamido)ethyl)benzoate

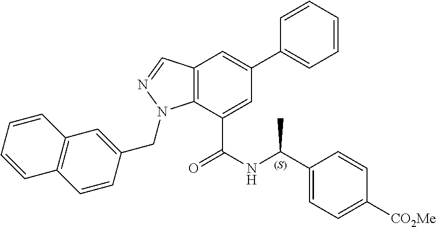

A mixture of the product of Step 3 (30 mg), phenylboronic acid (38 mg), Pd(dppf)₂Cl₂ (4 mg), K₂CO₃ (38 mg) in EtOH/H₂O (5 ml/1 mL) was stirred for 2 h at 70° C. under nitrogen. The reaction mixture was concentrated and purified by silica gel column chromatography (PE/EA=5:1) to afford the title compound as a yellow solid (27 mg). LCMS (ESI): 540.2 [M+1]⁺.

### Step 5 (S)-4-(1-(1-(naphthalen-2-ylmethyl)-5-phenyl-1H-indazole-7-carboxamido)ethyl)benzoic acid

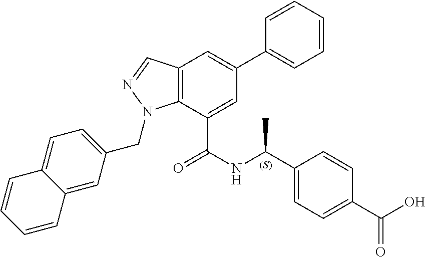

To a solution of the product of Step 4 (42 mg) in MeOH/H₂O (5 mL/0.5 mL) was added LiOH—H₂O (16 mg). The reaction mixture was stirred at 60° C. for 1 h. After being cooled to 0° C., the mixture was treated with 6 N HCl to adjusted pH≈6. The reaction mixture was then extracted with 3×20 mL EtOAc. The combined organic layer was washed with 20 mL brine, dried over anhydrous Na₂SO₄ and concentrated. The residue was purified by silica gel column chromatography (DCM/MeOH=10:1) to afford the title compound as a white solid (10.1 mg). LCMS (ESI): 526.2 [M+1]*. ¹HNMR (400 MHz, DMSO-d₆) δ 12.83 (s, 1H), 9.12 (d, J=7.6 Hz, 1H), 8.36 (s, 1H), 8.22 (s, 1H), 7.86-7.82 (m, 3H), 7.79-7.72 (m, 5H), 7.54-7.46 (m, 7H), 7.39 (t, J=7.4 Hz, 1H), 7.09 (d, J=8.5 Hz, 1H), 5.96 (s, 2H), 5.22 (p, J=7.1 Hz, 1H), 1.25 (s, 3H).

### EXAMPLE 168

(S)-4-(1-(5-(4-fluorophenyl)-1-(naphthalen-2-ylmethyl)-1H-indazole-7-carboxamido)ethyl)benzoic acid

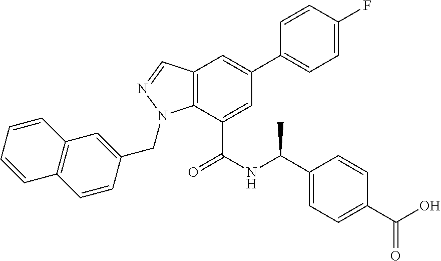 [SMILES: C[C@H](NC(=O)c1cc(-c2ccc(F)cc2)cc2cnn(Cc3ccc4ccccc4c3)c12)c1ccc(C(=O)O)cc1]

Starting with (4-fluorophenyl)boronic acid, the title product was obtained as a white solid by following the same procedures as described in Example 167. LCMS (ESI): 544.4[M+1]⁺.

¹HNMR (400 MHz, DMSO-d₆) δ 12.83 (s, 1H), 9.10 (d, J=7.6 Hz, 1H), 8.36 (s, 1H), 8.20 (s, 1H), 7.88-7.78 (m, 5H), 7.76-7.70 (m, 3H), 7.50-7.42 (m, 5H), 7.34 (t, J=8.8 Hz, 2H), 7.11-7.05 (m, 1H), 5.96 (s, 2H), 5.25-5.16 (m, 1H), 1.24 (d, J=6.9 Hz, 3H).

### EXAMPLE 169

### (S)-4-(1-(5-(3,5-difluorophenyl)-1-(naphthalen-2-ylmethyl)-1H-indazole-7-carboxamido)ethyl)benzoic acid

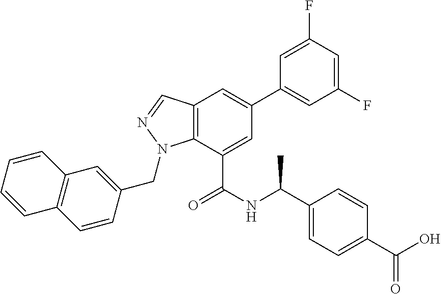 [SMILES: C[C@H](NC(=O)c1cc(-c2cc(F)cc(F)c2)cc2cnn(Cc3ccc4ccccc4c3)c12)c1ccc(C(=O)O)cc1]

Starting with (3,5-difluorophenyl)boronic acid, the title product was obtained as a white solid by following the same procedures as described in Example 167. LCMS (ESI): 562.6[M+1]⁺.

¹HNMR (400 MHz, DMSO-d₆) δ 12.83 (s, 1H), 9.06 (d, J=7.6 Hz, 1H), 8.38 (s, 1H), 8.34 (s, 1H), 7.88-7.78 (m, 4H), 7.76-7.69 (m, 2H), 7.60-7.53 (m, 2H), 7.51-7.41 (m, 5H), 7.30-7.20 (m, 1H), 7.11-7.02 (m, 1H), 5.96 (s, 2H), 5.24-5.16 (m, 1H), 1.26 (d, J=7.1 Hz, 3H).

### EXAMPLE 170

### (S)-4-(1-(1-(3-chlorobenzyl)-5-phenyl-1H-indazole-7-carboxamido)ethyl)benzoic acid

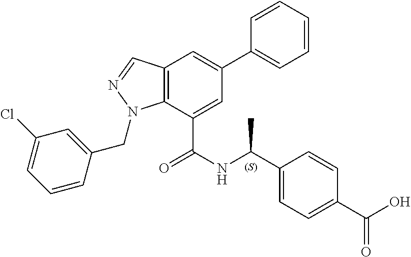

Starting with 1-(bromomethyl)-3-chlorobenzene, methyl (S)-4-(1-aminoethyl)benzoate and phenylboronic acid, the title product was obtained as a white solid by following the same procedures as described in Example 134. LCMS (ESI): 510.3 [M+1]⁺.

¹H NMR (400 MHz, DMSO-d₆) 12.85 (s, 1H), 9.26 (d, J=7.6 Hz, 1H), 8.35 (s, 1H), 8.23 (d, J=1.6 Hz, 1H), 7.90 (d, J=8.1 Hz, 2H), 7.84-7.76 (m, 3H), 7.56-7.47 (m, 4H), 7.40 (t, J=7.4 Hz, 1H), 7.31-7.25 (m, 1H), 6.95 (d, J=2.1 Hz, 1H), 6.84 (d, J=7.6 Hz, 1H), 5.78 (s, 2H), 5.24-5.14 (m, 1H), 1.41 (d, J=7.0 Hz, 3H).

### EXAMPLE 171

### 4-((5-phenyl-1-(4-(trifluoromethyl)benzyl)-1H-benzo[d]imidazole-7-carboxamido)methyl)benzoic acid

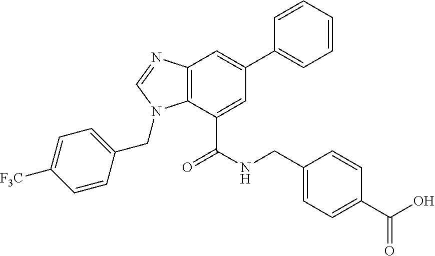 [SMILES: O=C(O)c1ccc(CNC(=O)c2cc(-c3ccccc3)cc3ncn(Cc4ccc(C(F)(F)F)cc4)c23)cc1]

### Step 1 methyl 5-bromo-1H-benzo[d]imidazole-7-carboxylate

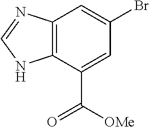 [SMILES: COC(=O)c1cc(Br)cc2nc[nH]c12]

To a mixture of methyl 2,3-diamino-5-bromobenzoate (1.0 g,) in MeCN (20 mL) at rt were added 1.2 eq Triethoxy methane and 10 mol % I₂. After stirring for 10 min at RT, the mixture was poured into 100 ml water. The brown solid was filtered and dried to afford the title compound (1.0 g). LCMS (ESI): 255 [M+1]⁺

### Step 2 methyl 5-bromo-1-(4-(trifluoromethyl)benzyl)-1H-benzo[d]imidazole-7-carboxylate

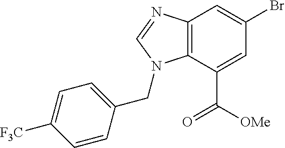 [SMILES: COC(=O)c1cc(Br)cc2ncn(Cc3ccc(C(F)(F)F)cc3)c12]

To a mixture of methyl 5-bromo-1H-benzo[d]imidazole-7-carboxylate (1.0 g,) and 1-(bromomethyl)-3-chlorobenzene in DMF (50 mL) at rt was added 2.0 eq Cs₂CO₃. After stirring for 4 h at rt, 200 mL was added. The reaction mixture was extracted 3×50 mL Ethyl acetate, washed with brine, dried over anhydrous Na₂SO₄ and filtered. The filtrate was concentrated and the residue was purified by silica gel chromatography to afford Isomer 1 as the title product (0.86 g, fast eluting) and Isomer 2.

Isomer 1: ¹H NMR (400 MHz, DMSO-d₆) δ 8.63 (s, 1H), 8.20 (d, J=2.0 Hz, 1H), 7.71 (d, J=2.0 Hz, 1H), 7.65 (d, J=8.1 Hz, 2H), 7.08 (d, J=8.0 Hz, 2H), 5.83 (s, 2H), 3.67 (s, 3H). LCMS (ESI): 415.289 [M+1]⁺. Isomer 2: ¹H NMR (400 MHz, DMSO) δ 8.64 (s, 1H), 8.16 (t, J=1.5 Hz, 1H), 7.85 (d, J=1.9 Hz, 1H), 7.73 (d, J=7.9 Hz, 2H), 7.49 (d, J=8.0 Hz, 2H), 5.71 (s, 2H), 3.91 (s, 3H). LCMS (ESI): 415.289 [M+1]⁺.

### Step 3 5-bromo-1-(4-(trifluoromethyl)benzyl)-1H-benzo[d]imidazole-7-carboxylic acid

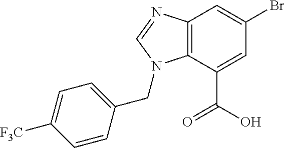 [SMILES: O=C(O)c1cc(Br)cc2ncn(Cc3ccc(C(F)(F)F)cc3)c12]

To a solution of the product of Step 1 (500 mg, 1.21 mmol) in MeOH/H₂O (10 mL/2.0 mL) was added LiOH—H₂O (254 mg, 6.06 mmol) at rt. The reaction mixture was stirred at 60° C. for 1 h. After being cooled to 0° C., the reaction mixture was treated with 6 N HCl to pH≈6. The reaction mixture was extracted 3 x 30 mL EtOAc. The combined organic layer washed with 20 mL brine, dried over anhydrous Na₂SO₄, concentrated. The residue was purified by silica gel column chromatography (DCM/MeOH: from 100:0 to 10:1) to afford the title compound as a white solid. LCMS (ESI): 399 [M+1]⁺.

### Step 4 methyl 4-((5-bromo-1-(4-(trifluoromethyl)benzyl)-1H-benzo[d]imidazole-7-carboxamido)methyl) benzoate

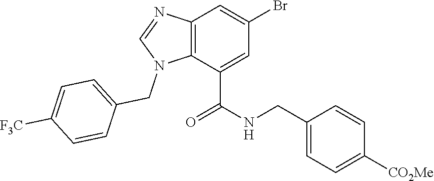 [SMILES: COC(=O)c1ccc(CNC(=O)c2cc(Br)cc3ncn(Cc4ccc(C(F)(F)F)cc4)c23)cc1]

To a mixture of the product of Step 3 (398 mg), methyl 4-(aminomethyl)benzoate (215 mg) and HATU (456 mg) in anhydrous DMF (100 mL) at 0° C. was added DIPEA (0.52 mL). After stirring at rt overnight, the reaction mixture was quenched with 100 mL water and extracted 3×100 mL EtOAc. The combined organic layer was washed with 100 mL brine, dried over anhydrous Na₂SO₄ and concentrated. The residue was purified by silica gel column chromatography (petroleum ether/ethyl acetate: from 100:0 to 5:1) to afford the title compound as a white solid (0.5 g). LCMS (ESI): 546 [M+1]⁺.

### Step 5 4-((5-phenyl-1-(4-(trifluoromethyl)benzyl)-1H-benzo[d]imidazole-7-carboxamido)-methyl)benzoic acid

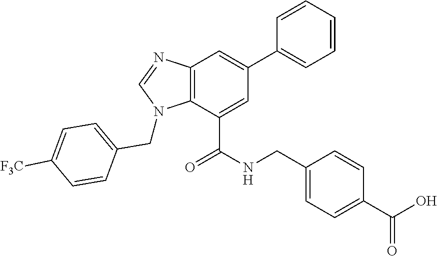 [SMILES: O=C(O)c1ccc(CNC(=O)c2cc(-c3ccccc3)cc3ncn(Cc4ccc(C(F)(F)F)cc4)c23)cc1]

Starting with the product of Step 4 and phenylboronic acid, the title product was obtained as a white solid by following the same procedures as described in Example 75. LCMS (ESI): 530 [M+1]⁺.

¹H NMR (400 MHz, CDCl₃) δ 9.11 (t, J=5.9 Hz, 1H), 8.58 (s, 1H), 8.11 (d, J=1.7 Hz, 1H), 7.86 (d, J=8.0 Hz, 2H), 7.77 (d, J=7.6 Hz, 2H), 7.70 (d, J=1.7 Hz, 1H), 7.55 (d, J=8.1 Hz, 2H), 7.48 (t, J=7.6 Hz, 2H), 7.36 (t, J=7.4 Hz, 1H), 7.31 (d, J=8.1 Hz, 2H), 7.10 (d, J=8.0 Hz, 2H), 5.84 (s, 2H), 4.44 (d, J=5.7 Hz, 2H).

### EXAMPLE 172

### 4-((5-(4-fluorophenyl)-1-(4-(trifluoromethyl)benzyl)-1H-benzo[d]imidazole-7-carboxamido)methyl)benzoic acid

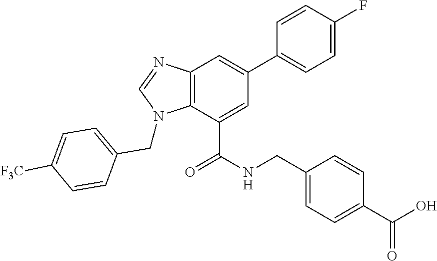 [SMILES: O=C(O)c1ccc(CNC(=O)c2cc(-c3ccc(F)cc3)cc3ncn(Cc4ccc(C(F)(F)F)cc4)c23)cc1]

Starting with the product of Step 4 of Example 171 and (4-fluorophenyl)boronic acid, the title product was obtained as a white solid by following the same procedures as described in Example 171. LCMS (ESI): 548 [M+1]⁺.

¹H NMR (400 MHz, DMSO-d₆) δ 12.87 (s, 1H), 9.11 (s, 1H), 8.60 (s, 1H), 8.11 (d, J=1.7 Hz, 1H), 7.89-7.78 (m, 4H), 7.69 (d, J=1.7 Hz, 1H), 7.55 (d, J=8.1 Hz, 2H), 7.36-7.27 (m, 4H), 7.11 (d, J=8.0 Hz, 2H), 5.85 (s, 2H), 4.45 (d, J=5.8 Hz, 2H).

### EXAMPLE 173

### 4-((5-(3,5-difluorophenyl)-1-(4-(trifluoromethyl)benzyl)-1H-benzo[d]imidazole-7-carboxamido)methyl)benzoic acid

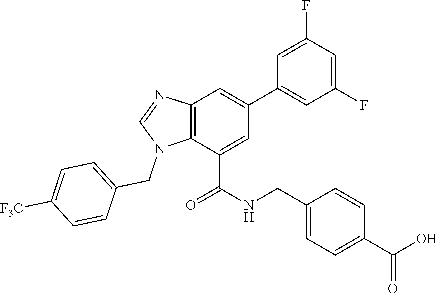 [SMILES: O=C(O)c1ccc(CNC(=O)c2cc(-c3cc(F)cc(F)c3)cc3ncn(Cc4ccc(C(F)(F)F)cc4)c23)cc1]

Starting with the product of Step 4 of Example 171 and (3,5-difluorophenyl)boronic acid, the title product was obtained as a white solid by following the same procedures as described in Example 171.

### LCMS (ESI): 566 [M+1]⁺.

¹H NMR (400 MHz, DMSO-d₆) δ 12.87 (s, 1H), 9.12 (t, J=5.9 Hz, 1H), 8.72 (d, J=2.1 Hz, 1H), 8.26 (d, J=1.7 Hz, 1H), 7.87 (d, J=8.0 Hz, 2H), 7.79 (d, J=1.8 Hz, 1H), 7.64-7.53 (m, 4H), 7.31 (d, J=8.0 Hz, 2H), 7.27-7.19 (m, 1H), 7.11 (d, J=8.0 Hz, 2H), 5.88 (s, 2H), 4.47 (d, J=5.8 Hz, 2H).

### EXAMPLE 174

### ((5-phenyl-1-(4-(trifluoromethyl)benzyl)-1H-benzo[d][1,2,3]triazole-7-carboxamido)methyl)benzoic acid

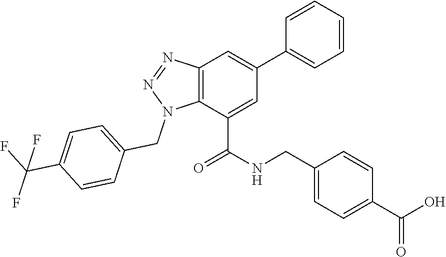 [SMILES: O=C(O)c1ccc(CNC(=O)c2cc(-c3ccccc3)cc3nnn(Cc4ccc(C(F)(F)F)cc4)c23)cc1]

### Step 1 5-bromo-3-nitro-2-((4-(trifluoromethyl)benzyl)amino)benzoic acid

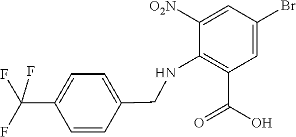 [SMILES: O=C(O)c1cc(Br)cc([N+](=O)[O-])c1NCc1ccc(C(F)(F)F)cc1]

A solution of 5-bromo-2-chloro-3-nitrobenzoic acid (600 mg, 2.14 mmol), DIPEA (1.1 g, 8.56 mmol) and (4-(trifluoromethyl)phenyl)methanamine (905 mg, 4.28 mmol) in 1,4-dioxane (6 mL) was stirred at 90° C. for 16 h. The reaction solution was poured into water and acidified with aqueous HCl (6N) till pH=4 and extracted with EA (30 mL×2). The combined organic layer was washed with 30 mL brine, dried over anhydrous Na₂SO₄, filtered and concentrated to afford the title compound as a brown solid (800 mg).

### LCMS (ESI): 419 [M+1]⁺.

### Step 2 5-bromo-1-(4-(trifluoromethyl)benzyl)-1H-benzo[d][1,2,3]triazole-7-carboxylic acid

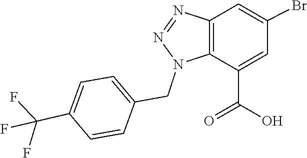 [SMILES: O=C(O)c1cc(Br)cc2nnn(Cc3ccc(C(F)(F)F)cc3)c12]

To a mixture of Zn (5.9 g, 89.47 mmol) in AcOH (20 mL) was added a solution of the product of Step 1 (800 mg, 1.91 mmol) in AcOH (10 mL) at 50° C. over 5 min. The reaction was stirred at 50° C. for 1 h. The mixture was cooled to 0° C., filtered and then washed with AcOH (5 mL). The organic layer was concentrated and then diluted with DCM (20 mL). To the above solution was added tert-butyl nitrite (197 mg, 1.91 mmol) at rt. The resulting solution was stirred at rt for 30 min and concentrated under vacuum.

The residue was dissolved into EA (50 mL) washed with water (pH=4), brine, dried over anhydrous Na₂SO₄ and filtered. The filtrate was concentrated under vacuum to afford the title compound as a brown solid (500 mg). LCMS (ESI): 400 [M+1]⁺.

### Step 3 methyl 4-((5-bromo-1-(4-(trifluoromethyl)benzyl)-1H-benzo[d][1,2,3]triazole-7-carboxamido)methyl)benzoate

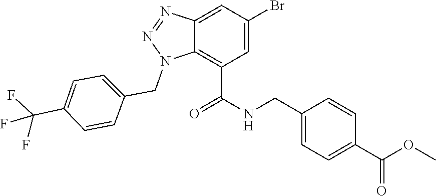 [SMILES: COC(=O)c1ccc(CNC(=O)c2cc(Br)cc3nnn(Cc4ccc(C(F)(F)F)cc4)c23)cc1]

To a solution of DIPEA (259 mg), the product of Step 2 (200 mg), methyl 4-(aminomethyl)benzoate hydrochloride (151 mg) in DMF (6 mL) were was added HATU (304 mg) at 0° C. After stirring at rt for 3 h, the reaction mixture was poured into water (40 mL) and extracted with EA (2×20 mL). The combine organic layer was washed with water, brine, dried over Na₂SO₄ and filtered. The filtrate was concentrated and the residue was stirred in MeOH (5 mL) at rt for 30 min. The solid was collected by filtration and washed with MeOH (3 mL) to afford title compound as a brown solid (180 mg). LCMS (ESI): 547 [M+1]⁺.

### Step 4 methyl 4-((5-phenyl-1-(4-(trifluoromethyl)benzyl)-1H-benzo[d][1,2,3]triazole-7-carboxamido)methyl)benzoate

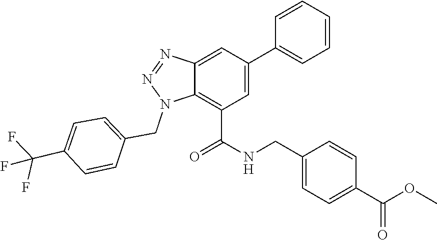 [SMILES: COC(=O)c1ccc(CNC(=O)c2cc(-c3ccccc3)cc3nnn(Cc4ccc(C(F)(F)F)cc4)c23)cc1]

A mixture of K₂CO₃ (45 mg), PdCl₂(dppf) (8.0 mg), Methyl 4-((5-bromo-1-(4-(trifluoromethyl)benzyl)-1H-benzo[d][1,2,3]triazole-7-carboxamido)methyl)benzoate (60 mg), phenylboronic acid (27 mg) in 1,4-dioxane/H₂O (1.8 mL, 5/1). The mixture was stirred at 70° C. for 2 h under Ar. The reaction mixture was concentrated and purified by silica gel column chromatography (PE/EA=5:1) to afford the title compound as a yellow solid (40 mg). LCMS (ESI): 545 [M+1]⁺.

### Step 5 4-((5-phenyl-1-(4-(trifluoromethyl)benzyl)-1H-benzo[d][1,2,3]triazole-7-carboxamido)methyl)benzoic acid

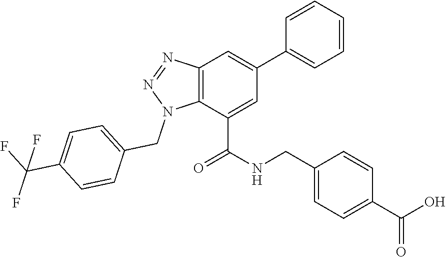 [SMILES: O=C(O)c1ccc(CNC(=O)c2cc(-c3ccccc3)cc3nnn(Cc4ccc(C(F)(F)F)cc4)c23)cc1]

To a solution of the product of Step 4 (40.0 mg) and LiOH·H₂O (31 mg) in THF/1,4-dioxane/H₂O (6 mL, 2/3/1) at rt. The resulting was stirred at 50° C. for 5 h. The reaction solution was poured into water and acidified with aqueous HCl (6N) until pH=6. The solid was collected by filtration and washed with water to afford the title compound as a light yellow solid (20 mg).LCMS (ESI): 531 [M+1]⁺.

¹HNMR (400 MHz, DMSO-d₆) δ 9.40 (t, J=6.0 Hz, 1H), 8.55 (s, 1H), 8.10 (s, 1H), 7.88 (t, J=7.1 Hz, 4H), 7.61-7.50 (m, 4H), 7.44 (t, J=7.4 Hz, 1H), 7.37 (d, J=8.0 Hz, 2H), 7.16 (d, J=8.0 Hz, 2H), 6.30 (s, 2H), 4.55 (d, J=5.9 Hz, 2H).

### EXAMPLE 175

### 4-(5-(4-fluorophenyl)-1-(4-(trifluoromethyl)benzyl)-1H-benzo[d][1,2,3]triazole-7-carboxamido)methyl)benzoic acid

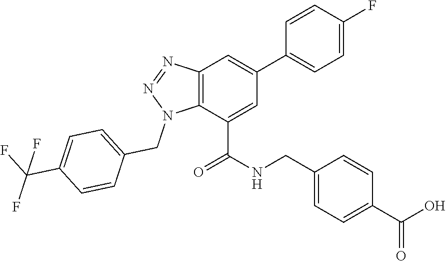 [SMILES: O=C(O)c1ccc(CNC(=O)c2cc(-c3ccc(F)cc3)cc3nnn(Cc4ccc(C(F)(F)F)cc4)c23)cc1]

Starting with the product of Step 4 of Example 174 and (4-fluorophenyl)boronic acid, the title product was obtained as a white solid by following the same procedures as described in Example 174. LCMS (ESI): 549.5 [M+1]⁺.

¹HNMR (400 MHz, DMSO-d₆) δ 12.88 (s, 1H), 9.39 (t, J=5.9 Hz, 1H), 8.54 (s, 1H), 8.07 (s, 1H), 7.98-7.91 (m, 2H), 7.90-7.86 (m, 2H), 7.56 (d, J=8.0 Hz, 2H), 7.38-7.35 (m, 6.5 Hz, 4H), 7.16 (d, J=8.0 Hz, 2H), 6.30 (s, 2H), 4.55 (d, J=5.8 Hz, 2H).

### EXAMPLE 176

### ((5-(3,5-difluorophenyl)-1-(4-(trifluoromethyl)benzyl)-1H-benzo[d][1,2,3]triazole-7-carboxamido)methyl)benzoic acid

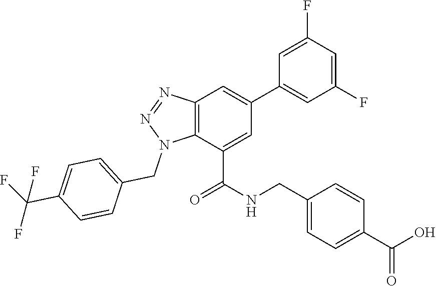 [SMILES: O=C(O)c1ccc(CNC(=O)c2cc(-c3cc(F)cc(F)c3)cc3nnn(Cc4ccc(C(F)(F)F)cc4)c23)cc1]

Starting with the product of Step 4 of Example 174 and (3,5-difluorophenyl)boronic acid, the title product was obtained as a white solid by following the same procedures as described in Example 174.

### LCMS (ESI): 567.5 [M+1]⁺.

¹HNMR (400 MHz, DMSO-d₆) δ 9.36 (t, J=6.0 Hz, 1H), 8.70 (s, 1H), 8.15 (s, 1H), 7.88 (d, J=8.0 Hz, 2H), 7.70 (d, J=8.0 Hz, 2H), 7.56 (d, J=8.0 Hz, 2H), 7.37 (d, J=8.0 Hz, 2H), 7.33-7.28 (m, 1H), 7.16 (d, J=8.0 Hz, 2H), 6.31 (s, 2H), 4.56 (d, J=5.8 Hz, 2H).

### EXAMPLE 177

### 44-((1-(naphthalen-1-ylmethyl)-5-phenyl-1H-indole-7-carboxamido)methyl)benzoic acid

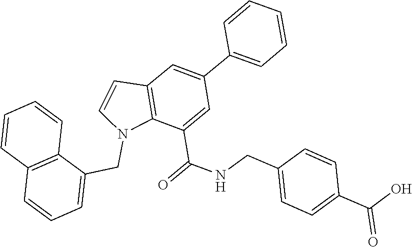 [SMILES: O=C(O)c1ccc(CNC(=O)c2cc(-c3ccccc3)cc3ccn(Cc4cccc5ccccc45)c23)cc1]

Starting with 1-(bromomethyl)naphthalene, and phenylboronic acid, the title product was obtained as a white solid by following the same procedures as described in Example 75. LCMS (ESI): 511.2 [M+1]⁺.

¹HNMR (400 MHz, DMSO-d₆) δ 12.75 (s, 1H), 8.91 (t, J=6.0 Hz, 1H), 8.08-8.04 (m, 2H), 8.01-7.96 (m, 1H), 7.83 (d, J=8.2 Hz, 1H), 7.76 (d, J=7.7 Hz, 2H), 7.62-7.53 (m, 5H), 7.52-7.45 (m, 3H), 7.35-7.28 (m, 2H), 6.92 (d, J=8.0 Hz, 2H), 6.75 (d, J=3.2 Hz, 1H), 6.32 (d, J=7.1 Hz, 1H), 6.15 (s, 2H), 4.07 (d, J=5.9 Hz, 2H).

### EXAMPLE 178

### (±) 4-((5-phenyl-1-(1-(4-(trifluoromethyl)phenyl)ethyl)-1H-indole-7-carboxamido)methyl)benzoic acid

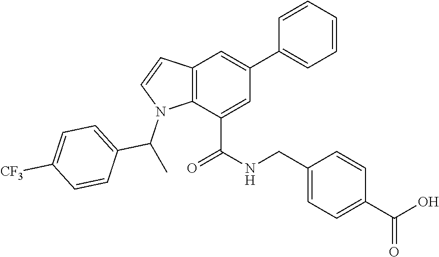 [SMILES: CC(c1ccc(C(F)(F)F)cc1)n1ccc2cc(-c3ccccc3)cc(C(=O)NCc3ccc(C(=O)O)cc3)c21]

Starting with 1-(1-chloroethyl)-4-(trifluoromethyl)benzene, phenylboronic acid, the title product was obtained as a white solid by following the same procedure described in Example 75. LCMS (ESI): 543.4 [M+1]⁺.

¹HNMR (400 MHz, DMSO-d₆) δ 9.17-9.06 (m, 1H), 7.99 (s, 1H), 7.89 (d, J=7.8 Hz, 2H), 7.77-7.69 (m, 3H), 7.55 (d, J=8.1 Hz, 2H), 7.51-7.44 (m, 3H), 7.41 (d, J=7.9 Hz, 2H), 7.34 (t, J=7.3 Hz, 1H), 7.09 (d, J=8.0 Hz, 2H), 6.76 (s, 1H), 6.32-6.23 (m, 1H), 4.62-4.55 (m, 1H), 4.47-4.39 (m, 1H), 1.82 (d, J=7.1 Hz, 3H).

### EXAMPLE 179

### (S)-4-(1-(5-phenyl-1-(4-(trifluoromethyl)benzyl)-1H-benzo[d][1,2,3]triazole-7-carboxamido)ethyl)benzoic acid

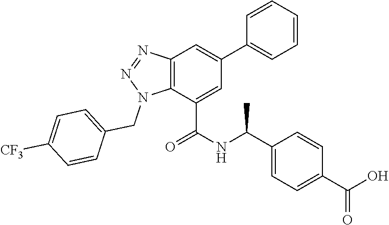 [SMILES: C[C@H](NC(=O)c1cc(-c2ccccc2)cc2nnn(Cc3ccc(C(F)(F)F)cc3)c12)c1ccc(C(=O)O)cc1]

Starting with methyl (S)-4-(1-aminoethyl)benzoate and the product of Step 2 of Example 174, the title product was obtained as a yellow solid by following the same procedure described in Example 174. LCMS (ESI): 545.5 [M+1]⁺.

¹HNMR (400 MHz, DMSO-d₆) δ 12.85 (s, 1H), 9.25 (d, J=7.8 Hz, 1H), 8.52 (s, 1H), 8.02 (s, 1H), 7.91 (d, J=7.9 Hz, 2H), 7.86 (d, J=7.7 Hz, 2H), 7.54 (t, J=7.6 Hz, 2H), 7.51-7.46 (m, 4H), 7.44 (t, J=7.3 Hz, 1H), 7.10 (d, J=7.9 Hz, 2H), 6.34-6.23 (m, 1H), 6.19-6.09 (m, 1H), 5.22-5.12 (m, 1H), 1.37 (d, J=7.0 Hz, 3H).

### EXAMPLE 180

### (S)-4-(1-(5-(4-fluorophenyl)-1-(4-(trifluoromethyl)benzyl)-1H-benzo[d][1,2,3]triazole-7-carboxamido)ethyl)benzoic acid

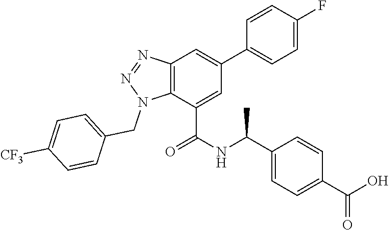 [SMILES: C[C@H](NC(=O)c1cc(-c2ccc(F)cc2)cc2nnn(Cc3ccc(C(F)(F)F)cc3)c12)c1ccc(C(=O)O)cc1]

Starting with (4-fluorophenyl)boronic acid, methyl (S)-4-(1-aminoethyl)benzoate and the product of Step 2 of Example 174, the title product was obtained as a yellow solid (36 mg) by following the same procedure described in Example 174. LCMS (ESI): 563.4 [M+1]⁺.

¹HNMR (400 MHz, DMSO-d₆) δ 12.85 (s, 1H), 9.24 (d, J=7.7 Hz, 1H), 8.52 (s, 1H), 8.00 (s, 1H), 7.95-7.86 (m, 4H), 7.52-7.44 (m, 4H), 7.41-7.33 (m, 2H), 7.10 (d, J=8.0 Hz, 2H), 6.30-6.24 (m, 1H), 6.18 6.10 (m, 1H), 5.22-5.12 (m, 1H), 1.37 (d, J=6.9 Hz, 3H).

### EXAMPLE 181

### (S)-4-(1-(5-(3,5-difluorophenyl)-1-(4-(trifluoromethyl)benzyl)-1H-benzo[d][1,2,3]triazole-7-carboxamido)ethyl)benzoic acid

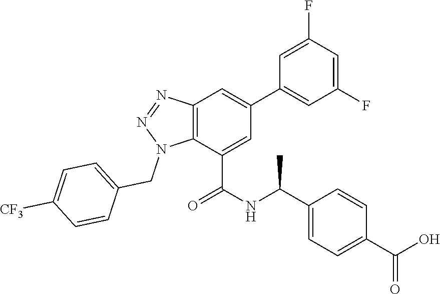 [SMILES: C[C@H](NC(=O)c1cc(-c2cc(F)cc(F)c2)cc2nnn(Cc3ccc(C(F)(F)F)cc3)c12)c1ccc(C(=O)O)cc1]

Starting with (3,5-difluorophenyl)boronic acid, methyl (S)-4-(1-aminoethyl)benzoate and the product of Step 2 of Example 174, the title product was obtained as a yellow solid by following the same procedure described in Example 174. LCMS (ESI): 581.5 [M+1]⁺.

¹HNMR (400 MHz, DMSO-d₆) δ 12.84 (s, 1H), 9.21 (d, J=7.8 Hz, 1H), 8.66 (s, 1H), 8.06 (s, 1H), 7.91 (d, J=7.9 Hz, 2H), 7.69 (d, J=7.9 Hz, 2H), 7.53-7.43 (m, 4H), 7.34-7.27 (m, 1H) 7.10 (d, J=7.9 Hz, 2H), 6.33-6.24 (m, 1H), 6.19-6.10 (m, 1H,, 5.22-5.12 (m, 1H), 1.40 (d, J=7.0 Hz, 3H).

### EXAMPLE 182

### 4-((4-((4-fluorophenyl)amino)-1-(naphthalen-2-ylmethyl)-1H-indazole-7-carboxamido)methyl)benzoic acid

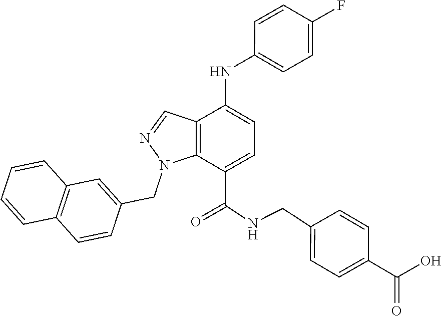 [SMILES: O=C(O)c1ccc(CNC(=O)c2ccc(Nc3ccc(F)cc3)c3cnn(Cc4ccc5ccccc5c4)c23)cc1]

Starting with methyl 4-bromo-1H-indazole-7-carboxylate, 2-(bromomethyl)naphthalene and methyl 4-(aminomethyl)benzoate, the title product was obtained as a white solid by following the same procedures described in Example 64. LCMS (ESI): 545.5 [M+1]⁺.

¹H NMR (400 MHz, DMSO-d₆) δ 12.82 (s, 1H), 8.89-8.82 (m, 1H), 8.81 (s, 1H), 8.45 (s, 1H), 7.87-7.80 (m, 1H), 7.79-7.67 (m, 4H), 7.51-7.43 (m, 3H), 7.40 (d, J=8.0 Hz, 1H), 7.35-7.28 (m, 4H), 7.22 (t, J=8.8 Hz, 2H), 7.13-7.08 (m, 1H), 6.63 (d, J=8.0 Hz, 1H), 6.01 (s, 2H), 4.50 (d, J=5.9 Hz, 2H).

### EXAMPLE 183

### ((4-((4-fluorophenyl)amino)-1-(naphthalen-2-ylmethyl)-1H-indole-7-carboxamido)methyl)benzoic acid

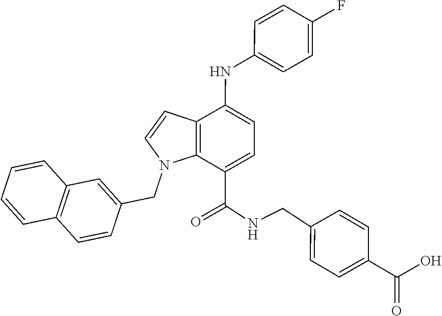 [SMILES: O=C(O)c1ccc(CNC(=O)c2ccc(Nc3ccc(F)cc3)c3ccn(Cc4ccc5ccccc5c4)c23)cc1]

Starting with 2-(bromomethyl)naphthalene and methyl 4-(aminomethyl)benzoate, the title product was obtained as a white solid by following the same procedures described in Example 64. LCMS (ESI): 544 [M+1]⁺.

¹H NMR (400 MHz, DMSO-d₆) δ 12.80 (s, 1H), 8.67 (t, J=6.0 Hz, 1H), 8.23 (s, 1H), 7.89-7.82 (m, 1H), 7.79-7.67 (m, 4H), 7.56-7.39 (m, 4H), 7.32-7.21 (m, 4H), 7.18-7.11 (m, 3H), 7.12-7.06 (m, 1H), 6.87 (d, J=3.2 Hz, 1H), 6.69 (d, J=8.0 Hz, 1H), 5.77 (s, 2H), 4.43 (d, J=5.9 Hz, 2H).

### EXAMPLE 184

### 4-((1-((4′-fluoro-[1,1′-biphenyl]-4-yl)methyl)-5-phenyl-1H-indazole-7-carboxamido)methyl)benzoic acid

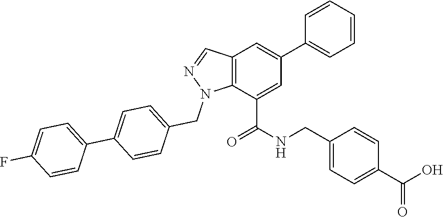 [SMILES: O=C(O)c1ccc(CNC(=O)c2cc(-c3ccccc3)cc3cnn(Cc4ccc(-c5ccc(F)cc5)cc4)c23)cc1]

Starting with methyl 5-bromo-1H-indazole-7-carboxylate, 4-(bromomethyl)-4′-fluoro-1,1′-biphenyl and phenylboronic acid, the title product was obtained as a white solid by following the same procedures as described in Example 75. LCMS (ESI): 556 [M+1]⁺.

¹H NMR (400 MHz, DMSO-d₆) δ 12.88 (s, 1H), 9.30 (d, J=6.1 Hz, 1H), 8.36 (s, 1H), 8.24 (d, J=1.7 Hz, 1H), 7.91-7.83 (m, 3H), 7.79 (d, J=7.4 Hz, 2H), 7.66-7.57 (m, 2H), 7.52 (t, J=7.7 Hz, 2H), 7.47-7.36 (m, 5H), 7.26 (t, J=8.9 Hz, 2H), 7.00 (d, J=8.0 Hz, 2H), 5.90 (s, 2H), 4.60 (d, J=5.9 Hz, 2H).

### EXAMPLE 185

### 4-((1-((4′-fluoro-[1,1′-biphenyl]-4-yl)methyl)-5-(4-fluorophenyl)-1H-indazole-7-carboxamido)methyl)benzoic acid

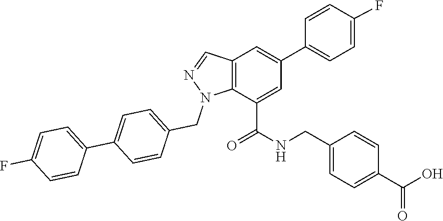 [SMILES: O=C(O)c1ccc(CNC(=O)c2cc(-c3ccc(F)cc3)cc3cnn(Cc4ccc(-c5ccc(F)cc5)cc4)c23)cc1]

Starting with methyl 5-bromo-1H-indazole-7-carboxylate, 4-(bromomethyl)-4′-fluoro-1,1′-biphenyl and (4-fluorophenyl)boronic acid, the title product was obtained as a white solid by following the same procedures as described in Example 75. LCMS (ESI): 574 [M+1]⁺.

¹H NMR (400 MHz, DMSO-d₆) δ 12.87 (s, 1H), 9.29 (t, J=6.0 Hz, 1H), 8.34 (s, 1H), 8.21 (d, J=1.7 Hz, 1H), 7.88-7.78 (m, 5H), 7.64-7.57 (m, 2H), 7.41 (t, J=8.4 Hz, 4H), 7.37-7.29 (m, 2H), 7.29-7.20 (m, 2H), 6.98 (d, J=8.3 Hz, 2H), 5.89 (s, 2H), 4.59 (d, J=5.9 Hz, 2H).

### EXAMPLE 186

### 4-((5-(3,5-difluorophenyl)-1-((4′-fluoro-[1,1′-biphenyl]-4-yl)methyl)-1H-indazole-7-carboxamido)methyl)benzoic acid

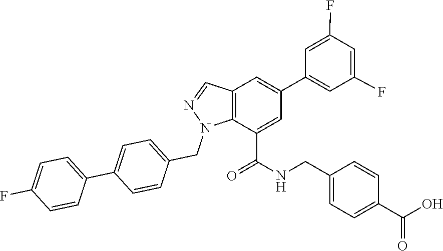 [SMILES: O=C(O)c1ccc(CNC(=O)c2cc(-c3cc(F)cc(F)c3)cc3cnn(Cc4ccc(-c5ccc(F)cc5)cc4)c23)cc1]

Starting with methyl 5-bromo-1H-indazole-7-carboxylate, 4-(bromomethyl)-4′-fluoro-1,1′-biphenyl and (3,5-difluorophenyl)boronic acid, the title product was obtained as a white solid by following the same procedures as described in Example 75. LCMS (ESI): 592 [M+1]⁺.

¹H NMR (400 MHz, DMSO-d₆) δ 12.88 (s, 1H), 9.29 (t, J=6.0 Hz, 1H), 8.38-8.33 (m, 2H), 7.93 (d, J=1.8 Hz, 1H), 7.87-7.82 (m, 2H), 7.64-7.54 (m, 4H), 7.45-7.36 (m, 4H), 7.29-7.20 (m, 3H), 6.96 (d, J=8.1 Hz, 2H), 5.90 (s, 2H), 4.60 (d, J=5.9 Hz, 2H).

### EXAMPLE 187

### 4-((5-(3,4-difluorophenyl)-1-((4′-fluoro-[1,1′-biphenyl]-4-yl)methyl)-1H-indazole-7-carboxamido)methyl)benzoic acid

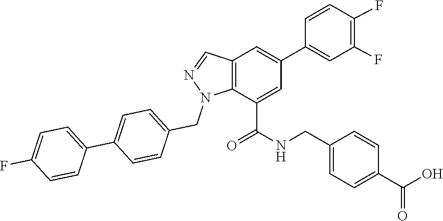 [SMILES: O=C(O)c1ccc(CNC(=O)c2cc(-c3ccc(F)c(F)c3)cc3cnn(Cc4ccc(-c5ccc(F)cc5)cc4)c23)cc1]

Starting with methyl 5-bromo-1H-indazole-7-carboxylate, 4-(bromomethyl)-4′-fluoro-1,1′-biphenyl and (3,4-difluorophenyl)boronic acid, the title product was obtained as a white solid by following the same procedures as described in Example 75. LCMS (ESI): 592 [M+1]⁺.

¹H NMR (400 MHz, DMSO-d₆) δ 12.86 (s, 1H), 9.27 (t, J=6.0 Hz, 1H), 8.35 (s, 1H), 8.27 (d, J=1.7 Hz, 1H), 7.93-7.82 (m, 4H), 7.68-7.51 (m, 4H), 7.41 (t, J=8.0 Hz, 4H), 7.29-7.20 (m, 2H), 6.97 (d, J=8.0 Hz, 2H), 5.89 (s, 2H), 4.60 (d, J=5.9 Hz, 2H).

### EXAMPLE 188

### 4-((1-([1,1′-biphenyl]-4-ylmethyl)-5-(3,4-difluorophenyl)-1H-indole-7-carboxamido)methyl)benzoic acid

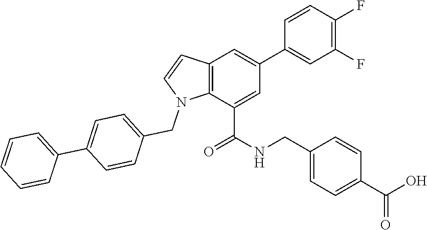 [SMILES: O=C(O)c1ccc(CNC(=O)c2cc(-c3ccc(F)c(F)c3)cc3ccn(Cc4ccc(-c5ccccc5)cc4)c23)cc1]

Starting with 4-(bromomethyl)-1,1′-biphenyl and (3,4-difluorophenyl)boronic acid, the title product was obtained as a white solid by following the same procedures as described in Example 75. LCMS (ESI): 573.5[M+1]⁺.

¹HNMR (400 MHz, DMSO-d₆) δ 12.84 (s, 1H), 9.02 (t, J=6.0 Hz, 1H), 8.03 (s, 1H), 7.87-7.76 (m, 3H), 7.65 (d, J=3.2 Hz, 1H), 7.61-7.49 (m, 5H), 7.48-7.39 (m, 4H), 7.38-7.29 (m, 3H), 6.93 (d, J=8.0 Hz, 2H), 6.71 (d, J=3.1 Hz, 1H), 5.65 (s, 2H), 4.51 (d, J=5.8 Hz, 2H).

### EXAMPLE 189

### (S)-4-(1-(5-phenyl-1-(4-(trifluoromethyl)benzyl)-1H-indole-7-carboxamido)ethyl) benzoic acid

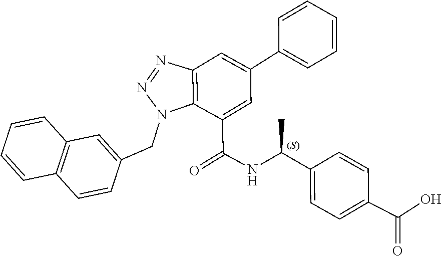

Starting with naphthalen-2-ylmethanamine and methyl (S)-4-(1-aminoethyl)benzoate, the title product was obtained as a white solid by following the same procedures as described in Example 174. LCMS (ESI): 527.5 [M+1]⁺.

¹HNMR (400 MHz, DMSO-d₆) δ 8.92 (d, J=7.8 Hz, 1H), 8.02-8.00 (m, 1H), 7.90-7.83 (m, 2H), 7.78-7.71 (m, 2H), 7.62 (d, J=3.2 Hz, 1H), 7.53-7.45 (m, 5H), 7.45-7.41 (m, 2H), 7.37-7.32 (m, 1H), 6.95 (d, J=8.0 Hz, 2H), 6.78-6.70 (m, 1H), 5.65 (s, 2H), 5.08-5.00 (m, 1H), 1.26-1.21 (m, 3H).

### EXAMPLE 190

### 4-((1-([1,1′-biphenyl]-4-ylmethyl)-5-phenyl-1H-indazole-7-carboxamido)methyl)benzoic acid

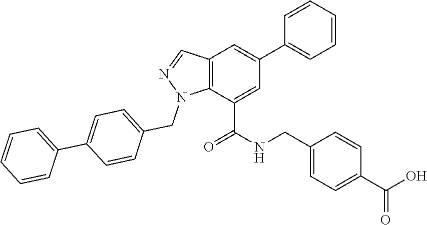 [SMILES: O=C(O)c1ccc(CNC(=O)c2cc(-c3ccccc3)cc3cnn(Cc4ccc(-c5ccccc5)cc4)c23)cc1]

Starting with methyl 5-bromo-1H-indazole-7-carboxylate, 4-(bromomethyl)-1,1′-biphenyl and phenylboronic acid, the title product was obtained as a white solid (20 mg) by following the same procedures described in Example 134. LCMS (ESI): 538.3 [M+1]⁺.

¹HNMR (400 MHz, DMSO-d₆) δ 12.88 (s, 1H), 9.32 (t, J=6.0 Hz, 1H), 8.36 (s, 1H), 8.24 (d, J=1.7 Hz, 1H), 7.92-7.74 (m, 5H), 7.62-7.32 (m, 12H), 7.01 (d, J=8.0 Hz, 2H), 5.90 (s, 2H), 4.61 (d, J=5.9 Hz, 2H).

### EXAMPLE 191

### 4-((1-([1,1′-biphenyl]-4-ylmethyl)-5-(4-fluorophenyl)-1H-indazole-7-carboxamido)methyl)benzoic acid

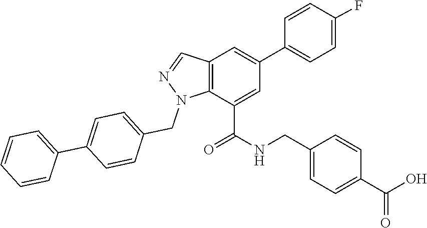 [SMILES: O=C(O)c1ccc(CNC(=O)c2cc(-c3ccc(F)cc3)cc3cnn(Cc4ccc(-c5ccccc5)cc4)c23)cc1]

Starting with methyl 5-bromo-1H-indazole-7-carboxylate, 4-(bromomethyl)-1,1′-biphenyl and (4-fluorophenyl)boronic acid, the title product was obtained as a white solid by following the same procedures described in Example 134. LCMS (ESI): 556.5 [M+1]⁺.

¹H NMR (400 MHz, DMSO-d₆) δ 12.87 (s, 1H), 9.31 (t, J=6.0 Hz, 1H), 8.36 (s, 1H), 8.22 (d, J=1.7 Hz, 1H), 7.93-7.74 (m, 5H), 7.62-7.58 (m, 2H), 7.49-7.41 (m, 6H), 7.38-7.30 (m, 3H), 7.00 (d, J=8.0 Hz, 2H), 5.89 (s, 2H), 4.60 (d, J=5.8 Hz, 2H).

### EXAMPLE 192

### 4-((1-([1,1′-biphenyl]-4-ylmethyl)-5-(3,5-difluorophenyl)-1H-indazole-7-carboxamido)methyl)benzoic acid

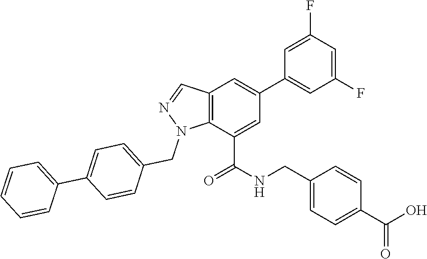 [SMILES: O=C(O)c1ccc(CNC(=O)c2cc(-c3cc(F)cc(F)c3)cc3cnn(Cc4ccc(-c5ccccc5)cc4)c23)cc1]

Starting with methyl 5-bromo-1H-indazole-7-carboxylate, 4-(bromomethyl)-1,1′-biphenyl and (3,5-difluorophenyl)boronic acid, the title product was obtained as a white solid by following the same procedures described in Example 134. LCMS (ESI): 574.5 [M+1]⁺.

¹H NMR (400 MHz, DMSO-d₆) δ 12.88 (s, 1H), 9.30 (t, J=5.9 Hz, 1H), 8.40-8.35 (m, 2H), 7.94 (d, J=1.8 Hz, 1H), 7.88 (d, J=8.0 Hz, 2H), 7.63-7.54 (m, 4H), 7.47-7.41 (m, 6H), 7.35 (t, J=7.3 Hz, 1H), 7.28-7.21 (m, 1H), 6.99 (d, J=8.0 Hz, 2H), 5.91 (s, 2H), 4.62 (d, J=5.9 Hz, 2H).

### EXAMPLE 193

### 4-((1-([1,1′-biphenyl]-4-ylmethyl)-5-(3,4-difluorophenyl)-1H-indazole-7-carboxamido)methyl)benzoic acid

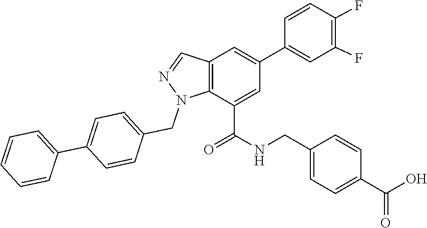 [SMILES: O=C(O)c1ccc(CNC(=O)c2cc(-c3ccc(F)c(F)c3)cc3cnn(Cc4ccc(-c5ccccc5)cc4)c23)cc1]

Starting with methyl 5-bromo-1H-indazole-7-carboxylate, 4-(bromomethyl)-1,1′-biphenyl and (3,4-difluorophenyl)boronic acid, the title product was obtained as a white solid by following the same procedures described in Example 134. LCMS (ESI): 574.5 [M+1]⁺.

¹H NMR (400 MHz, DMSO-d₆) δ 12.89 (s, 1H), 9.31 (d, J=5.9 Hz, 1H), 8.37 (s, 1H), 8.28 (d, J=1.7 Hz, 1H), 7.95-7.84 (m, 4H), 7.71-7.51 (m, 4H), 7.44 (t, J=7.4 Hz, 6H), 7.37-7.33 (m, 1H), 7.00 (d, J=8.0 Hz, 2H), 5.90 (s, 2H), 4.61 (d, J=5.8 Hz, 2H).

### EXAMPLE 194

### (S)-4-(1-(5-(4-fluorophenyl)-1-(4-(trifluoromethyl)benzyl)-1H-indazole-7-carboxamido)ethyl)benzoic acid

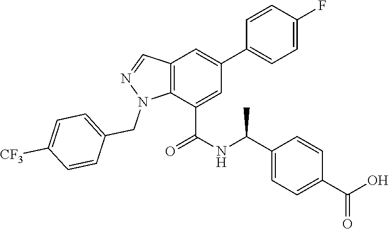 [SMILES: C[C@H](NC(=O)c1cc(-c2ccc(F)cc2)cc2cnn(Cc3ccc(C(F)(F)F)cc3)c12)c1ccc(C(=O)O)cc1]

Starting with 1-(bromomethyl)-4-(trifluoromethyl)benzene, the title product was obtained as a white solid by following the same procedures as described in Example 134. LCMS (ESI): 562.5[M+1]⁺.

¹HNMR (400 MHz, DMSO-d₆) δ 12.85 (s, 1H), 9.19 (d, J=7.8 Hz, 1H), 8.36 (s, 1H), 8.22 (d, J=1.7 Hz, 1H), 7.90 (d, J=8.0 Hz, 2H), 7.86-7.81 (m, 2H), 7.80 (d, J=1.7 Hz, 1H), 7.49 (d, J=7.9 Hz, 4H), 7.36 (t, J=8.8 Hz, 2H), 7.03 (d, J=8.0 Hz, 2H), 5.98-5.77 (m, 2H), 5.22-5.12 (m, 1H), 1.37 (d, J=7.1 Hz, 3H).

### EXAMPLE 195

### (S)-4-(1-(5-(3,4-difluorophenyl)-1-(4-(trifluoromethyl)benzyl)-1H-indazole-7-carboxamido)ethyl)benzoic acid

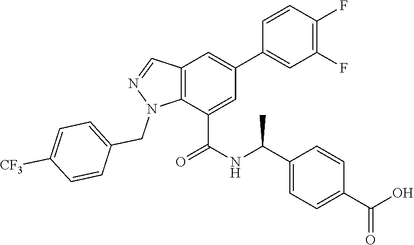 [SMILES: C[C@H](NC(=O)c1cc(-c2ccc(F)c(F)c2)cc2cnn(Cc3ccc(C(F)(F)F)cc3)c12)c1ccc(C(=O)O)cc1]

Starting with 1-(bromomethyl)-4-(trifluoromethyl)benzene and (3,4-difluorophenyl)boronic acid, the title product was obtained as a white solid (76 mg) by following the same procedures as described in Example 134. LCMS (ESI): 580.6[M+1]⁺.

¹HNMR (400 MHz, DMSO-d₆) δ 12.83 (s, 1H), 9.16 (d, J=7.8 Hz, 1H), 8.35 (s, 1H), 8.26 (d, J=1.7 Hz, 1H), 7.94-7.84 (m, 3H), 7.81 (d, J=1.8 Hz, 1H), 7.68-7.61 (m, 1H), 7.60-7.53 (m, 1H), 7.51-7.43 (m, 4H), 7.01 (d, J=8.0 Hz, 2H), 5.95-5.77 (m, 2H), 5.20-5.10 (m, 1H), 1.36 (d, J=7.2 Hz, 3H).

### EXAMPLE 196

### 4-((S)-1-(1-((R)-1-(naphthalen-2-yl)ethyl)-5-phenyl-1H-indazole-7-carboxamido)ethyl)benzoic acid

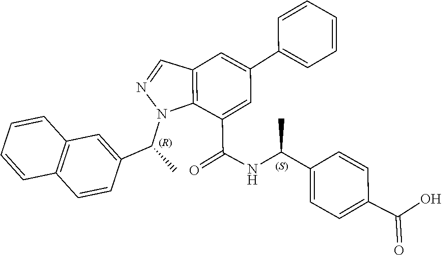

### Step 1 methyl (R)-5-bromo-1-(1-(naphthalen-2-yl)ethyl)-1H-indazole-7-carboxylate

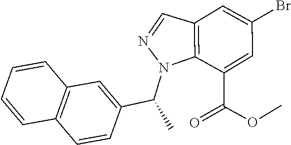 [SMILES: COC(=O)c1cc(Br)cc2cnn([C@H](C)c3ccc4ccccc4c3)c12]

A mixture methyl 5-bromo-1H-indazole-7-carboxylate (300 mg, 1.18 mmol), (S)-1-(naphthalen-2-yl)ethan-1-ol (304 mg, 1.76 mmol) was degassed and purged with Ar for three times. To the above mixture was added THF (10 mL), TBP (428 mg, 2.11 mmol) and dropwise DIAD (357 mg, 1.76 mmol) successively at 0° C. The resulting was stirred at rt for 2 h. The reaction solution was concentrated and the residue was purified using silica gel column chromatography with EA/PE (0 to 20%) to afford the title compound (270 mg) as a colorless oil. LCMS (ESI): 409.5 [M+1]⁺.

¹H NMR (400 MHz, CDCl₃) δ 8.15 (s, 1H), 8.04 (d, J=2.0 Hz, 1H), 7.85 (d, J=2.0 Hz, 1H), 7.78-7.64 (m, 3H), 7.46 (s, 1H), 7.45-7.37 (m, 2H), 7.11 (d, J=8.5, 1H), 6.66 (q, J=6.9 Hz, 1H), 3.86 (s, 3H), 2.11 (d, J=6.9 Hz, 3H).

### Step 2 4—((S)-1-(1-((R)-1-(naphthalen-2-yl)ethyl)-5-phenyl-1H-indazole-7-carboxamido)ethyl)benzoic acid

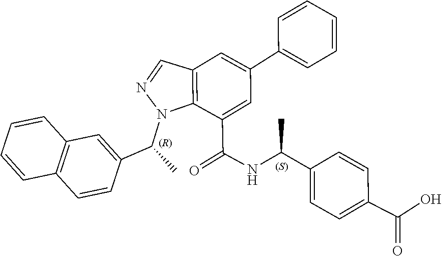

Starting with the product of Step 1 (200 mg), the title product was obtained as a white solid (60 mg) by following the same procedure described for Example 167. LCMS (ESI): 540.5[M+1]⁺.

¹H NMR (400 MHz, DMSO-d₆) δ 9.34 (d, J=8.2 Hz, 1H), 8.36 (s, 1H), 8.20 (s, 1H), 7.87-7.74 (m, 6H), 7.59 (d, J=8.6 Hz, 1H), 7.50 (d, J=13.8, 5H), 7.46-7.35 (m, 4H), 7.10 (d, J=8.5 Hz, 1H), 6.45 (q, J=6.8 Hz, 1H), 5.43-5.35 (m, 1H), 1.97 (d, J=6.9 Hz, 3H), 1.58 (d, J=6.9 Hz, 3H).

### EXAMPLE 197

### 4-((S)-1-(1-((S)-1-(naphthalen-2-yl)ethyl)-5-phenyl-1H-indazole-7-carboxamido)ethyl)benzoic acid

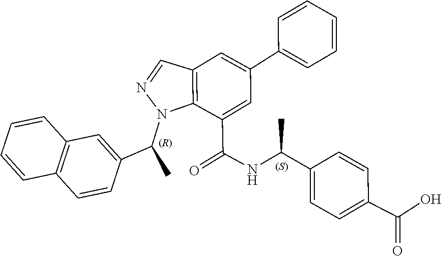

Starting with (R)-1-(naphthalen-2-yl)ethan-1-ol, the title product was obtained as a white solid by following the same procedure described for Example 196. LCMS (ESI): 540.5[M+1]⁺.

¹H NMR (400 MHz, DMSO-d₆) δ 12.86 (s, 1H), 9.18 (d, J=7.3 Hz, 1H), 8.39 (s, 1H), 8.20 (d, J=1.7 Hz, 1H), 7.99 (d, J=8.2 Hz, 2H), 7.88-7.82 (m, 2H), 7.80 (d, J=8.6 Hz, 1H), 7.77-7.72 (m, 2H), 7.69 (d, J=1.7 Hz, 1H), 7.62 (d, J=8.3 Hz, 2H), 7.57-7.44 (m, 5H), 7.43-7.32 (m, 1H), 7.19 (s, 1H), 6.46-6.36 (m, 1H), 5.35-5.24 (m, 1H), 1.94 (d, J=6.9 Hz, 3H), 1.27 (d, J=7.1 Hz, 3H).

### EXAMPLE 198

### (S)-4-(1-(1-([1,1′-biphenyl]-4-ylmethyl)-5-phenyl-1H-indazole-7-carboxamido)ethyl)benzoic acid

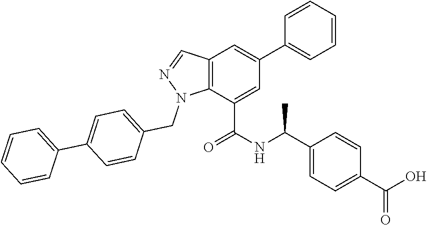 [SMILES: C[C@H](NC(=O)c1cc(-c2ccccc2)cc2cnn(Cc3ccc(-c4ccccc4)cc3)c12)c1ccc(C(=O)O)cc1]

Starting with 4-(bromomethyl)-1,1′-biphenyl and phenylboronic acid, the title product was obtained as a white solid (23 mg) by following the same procedures described in Example 134. LCMS (ESI): 552.5 [M+1]⁺.

¹H NMR (400 MHz, DMSO-d₆) δ 12.87 (s, 1H), 9.24 (d, J=7.9 Hz, 1H), 8.35 (s, 1H), 8.22 (d, J=1.7 Hz, 1H), 7.93 (d, J=8.0 Hz, 2H), 7.87-7.75 (m, 3H), 7.58-7.50 (m, 6H), 7.47-7.32 (m, 6H), 6.98 (d, J=8.1 Hz, 2H), 5.92-5.74 (m, 2H), 5.29-5.25 (m, 1H), 1.44 (d, J=7.0 Hz, 3H).

### EXAMPLE 199

### (S)-4-(1-(1-([1,1′-biphenyl]-4-ylmethyl)-5-(4-fluorophenyl)-1H-indazole-7-carboxamido)ethyl)benzoic acid

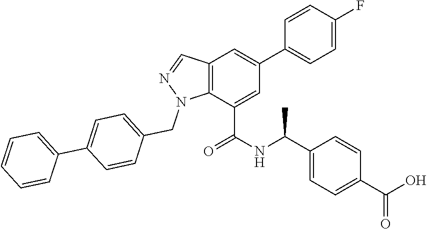 [SMILES: C[C@H](NC(=O)c1cc(-c2ccc(F)cc2)cc2cnn(Cc3ccc(-c4ccccc4)cc3)c12)c1ccc(C(=O)O)cc1]

Starting with 4-(bromomethyl)-1,1′-biphenyl, the title product was obtained as a white solid by following the same procedures described in Example 134. LCMS (ESI): 570.5 [M+1]⁺.

¹H NMR (400 MHz, DMSO-d₆) δ 12.84 (s, 1H), 9.22 (d, J=7.9 Hz, 1H), 8.34 (s, 1H), 8.21 (d, J=1.7 Hz, 1H), 7.93 (d, J=8.3 Hz, 2H), 7.86-7.78 (m, 3H), 7.60-7.53 (m, 4H), 7.49-7.32 (m, 7H), 6.98 (d, J=8.1 Hz, 2H), 5.91-5.72 (m, 2H), 5.32-5.27 (m, 1H,, 1.44 (d, J=7.0 Hz, 3H).

### EXAMPLE 200

### (S)-4-(1-(1-([1,1′-biphenyl]-4-ylmethyl)-5-(3,5-difluorophenyl)-1H-indazole-7-carboxamido)ethyl)benzoic acid

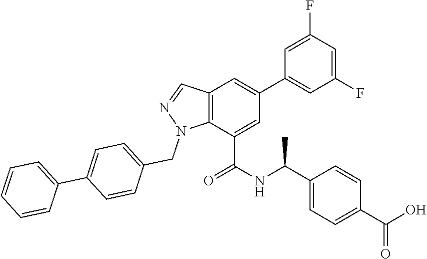 [SMILES: C[C@H](NC(=O)c1cc(-c2cc(F)cc(F)c2)cc2cnn(Cc3ccc(-c4ccccc4)cc3)c12)c1ccc(C(=O)O)cc1]

Starting with 4-(bromomethyl)-1,1′-biphenyl and (3,5-difluorophenyl)boronic acid, the title product was obtained as a white solid (23 mg) by following the same procedures described in Example 134. LCMS (ESI): 588.5 [M+1]⁺.

¹H NMR (400 MHz, DMSO-d₆) δ 12.88 (s, 1H), 9.20 (d, J=7.9 Hz, 1H), 8.40-8.31 (m, 2H), 7.93 (d, J=8.2 Hz, 2H), 7.87 (d, J=1.7 Hz, 1H), 7.63-7.53 (m, 6H), 7.48-7.33 (m, 5H), 7.30-7.25 (m, 1H), 6.97 (d, J=8.1 Hz, 2H), 5.93-5.75 (m, 2H), 5.33-5.27 (m, 1H), 1.46 (d, J=7.0 Hz, 3H).

### EXAMPLE 201

### (S)-4-(1-(1-([1,1′-biphenyl]-4-ylmethyl)-5-(3,4-difluorophenyl)-1H-indazole-7-carboxamido)ethyl)benzoic acid

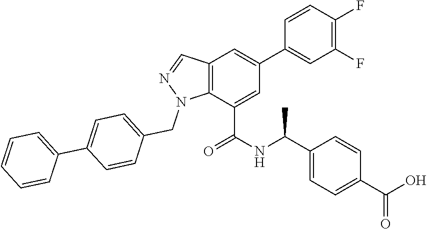 [SMILES: C[C@H](NC(=O)c1cc(-c2ccc(F)c(F)c2)cc2cnn(Cc3ccc(-c4ccccc4)cc3)c12)c1ccc(C(=O)O)cc1]

Starting with 4-(bromomethyl)-1,1′-biphenyl and (3,4-difluorophenyl)boronic acid, the title product was obtained as a white solid by following the same procedures described in Example 134. LCMS (ESI): 588.4 [M+1]⁺.

¹H NMR (400 MHz, DMSO-d₆) δ 12.88 (s, 1H), 9.20 (d, J=7.9 Hz, 1H), 8.36 (s, 1H), 8.27 (d, J=1.7 Hz, 1H), 7.97-7.87 (m, 3H), 7.82 (d, J=1.7 Hz, 1H), 7.69-7.53 (m, 6H), 7.48-7.31 (m, 5H), 6.97 (d, J=8.0 Hz, 2H), 5.91-5.74 (m, 2H), 5.31-5.27 (m, 1H), 1.45 (d, J=7.0 Hz, 3H).

### EXAMPLE 202

### (S)-4-(1-(1-((4′-fluoro-[1,1′-biphenyl]-4-yl)methyl)-5-phenyl-1H-indazole-7-carboxamido)ethyl)benzoic acid

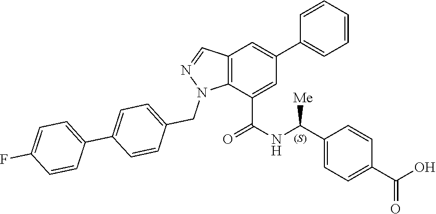

Starting with 4-(bromomethyl)-4′-fluoro-1,1′-biphenyl and phenylboronic acid, phenylboronic acid, the title product was obtained as a white solid by following the same procedures as described in Example 134.

### LCMS (ESI): 570 [M+1]⁺.

¹H NMR (400 MHz, DMSO-d₆) δ 12.88 (s, 1H), 9.23 (d, J=7.9 Hz, 1H), 8.35 (s, 1H), 8.22 (d, J=1.7 Hz, 1H), 7.92 (d, J=8.0 Hz, 2H), 7.85-7.76 (m, 3H), 7.63-7.49 (m, 6H), 7.45-7.34 (m, 3H), 7.26 (t, J=8.8 Hz, 2H), 6.97 (d, J=8.0 Hz, 2H), 5.87 (d, J=15.7 Hz, 1H), 5.77 (d, J=15.7 Hz, 1H), 5.35-5.24 (m, 1H), 1.44 (d, J=7.0 Hz, 3H).

### EXAMPLE 203

### (S)-4-(1-(1-((4′-fluoro-[1,1′-biphenyl]-4-yl)methyl)-5-(4-fluorophenyl)-1H-indazole-7-carboxamido)ethyl)benzoic acid

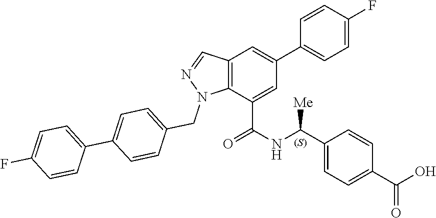

Starting with 4-(bromomethyl)-4′-fluoro-1,1′-biphenyl, the title product was obtained as a white solid by following the same procedures as described in Example 134. LCMS (ESI): 588 [M+1]⁺.

¹H NMR (400 MHz, DMSO-d₆) δ 12.89 (s, 1H), 9.22 (d, J=7.9 Hz, 1H), 8.34 (s, 1H), 8.21 (d, J=1.7 Hz, 1H), 7.92 (d, J=8.3 Hz, 2H), 7.88-7.78 (m, 3H), 7.64-7.51 (m, 4H), 7.42-7.19 (m, 4H), 6.97 (d, J=8.0 Hz, 2H), 5.87 (d, J=15.7 Hz, 1H), 5.77 (d, J=15.7 Hz, 1H), 5.35-5.23 (m, 1H), 1.44 (d, J=7.0 Hz, 3H).

### EXAMPLE 204

### (S)-4-(1-(5-(3,5-difluorophenyl)-1-((4′-fluoro-[1,1′-biphenyl]-4-yl)methyl)-1H-indazole-7-carboxamido)ethyl)benzoic acid

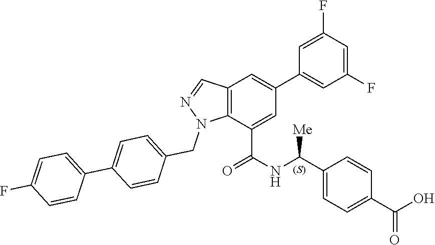

Starting with 4-(bromomethyl)-4′-fluoro-1,1′-biphenyl and (3,5-difluorophenyl)boronic acid, the title product was obtained as a white solid by following the same procedures as described in Example 134.

### LCMS (ESI): 606 [M+1]⁺.

¹H NMR (400 MHz, DMSO-d₆) δ 12.89 (s, 1H), 9.19 (s, 1H), 8.39-8.32 (m, 2H), 7.94-7.85 (m, 3H), 7.63-7.51 (m, 6H), 7.37 (d, J=8.0 Hz, 2H), 7.26 (t, J=8.6 Hz, 3H), 6.96 (d, J=8.0 Hz, 2H), 5.88 (d, J=15.7 Hz, 1H), 5.76 (d, J=15.6 Hz, 1H), 5.29 (t, J=7.2 Hz, 1H), 1.46 (d, J=7.0 Hz, 5H).

### EXAMPLE 205

### (S)-4-(1-(5-(3,4-difluorophenyl)-1-((4′-fluoro-[1,1′-biphenyl]-4-yl)methyl)-1H-indazole-7-carboxamido)ethyl)benzoic acid

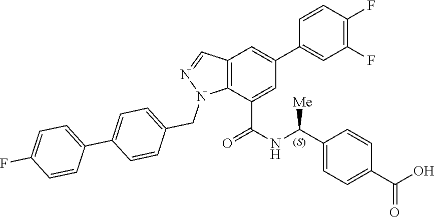

Starting with 4-(bromomethyl)-4′-fluoro-1,1′-biphenyl and (3,4-difluorophenyl)boronic acid, the title product was obtained as a white solid by following the same procedures as described in Example 134.

### LCMS (ESI): 606 [M+1]⁺.

¹H NMR (400 MHz, DMSO-d₆) δ 12.87 (s, 1H), 9.19 (d, J=7.9 Hz, 1H), 8.35 (s, 1H), 8.26 (d, J=1.7 Hz, 1H), 7.91 (d, J=8.1 Hz, 3H), 7.83 (d, J=1.7 Hz, 1H), 7.67-7.51 (m, 6H), 7.37 (d, J=8.1 Hz, 2H), 7.26 (t, J=8.8 Hz, 2H), 6.96 (d, J=8.0 Hz, 2H), 5.87 (d, J=15.7 Hz, 1H), 5.76 (d, J=15.7 Hz, 1H), 5.29 (t, J=7.4 Hz, 1H), 1.45 (d, J=7.0 Hz, 3H).

### EXAMPLE 206

### 4-(1-(1-(3-chlorobenzyl)-5-(3-cyanophenyl)-1H-indole-7-carboxamido)cyclopropyl)benzoic acid

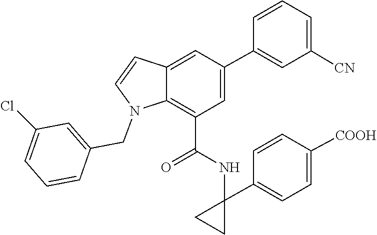 [SMILES: N#Cc1cccc(-c2cc(C(=O)NC3(c4ccc(C(=O)O)cc4)CC3)c3c(ccn3Cc3cccc(Cl)c3)c2)c1]

Starting with 1-(bromomethyl)-3-chlorobenzene, methyl 4-(1-aminocyclopropyl)benzoate and (3-cyanophenyl)phenylboronic acid, the title product was obtained as a white solid by following the same procedures as described in Example 75. LCMS (ESI): 546 [M+1]⁺. 400 MHz H NMR (400 MHz, DMSO-d₆) δ 12.77 (s, 1H), 9.37 (s, 1H), 8.30 (s, 1H), 8.17 (d, J=6.0 Hz, 2H), 7.85-7.78 (m, 3H), 7.78-7.68 (m, 2H), 7.55 (d, J=3.2 Hz, 1H), 7.34-7.24 (m, 4H), 6.88 (s, 1H), 5.66 (s, 2H), 1.31-1.22 (m, 3H), 1.14-1.08 (m, 2H).

### EXAMPLE 207

### (S)-4-(1-(1-([1,1′-biphenyl]-4-ylmethyl)-5-(3,4-difluorophenyl)-1H-indole-7-carboxamido)ethyl)benzoic acid

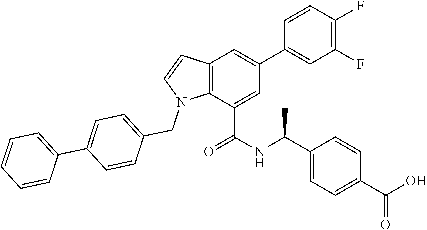 [SMILES: C[C@H](NC(=O)c1cc(-c2ccc(F)c(F)c2)cc2ccn(Cc3ccc(-c4ccccc4)cc3)c12)c1ccc(C(=O)O)cc1]

Starting with 4-(bromomethyl)-1,1′-biphenyl, methyl (S)-4-(1-aminoethyl)benzoate and (3,4-difluorophenyl) boronic acid, the title product was obtained as a white solid by following the same procedures as described in Example 75. LCMS (ESI): 587.4 [M+1]⁺.

¹HNMR (400 MHz, DMSO-d₆) δ 12.86 (s, 1H), 8.96 (d, J=7.8 Hz, 1H), 8.04 (s, 1H), 7.90 (d, J=8.0 Hz, 2H), 7.87-7.80 (m, 1H), 7.67 (d, J=3.2 Hz, 1H), 7.61-7.50 (m, 7H), 7.47-7.41 (m, 4H), 7.37-7.32 (m, 1H), 6.93 (d, J=7.9 Hz, 2H), 6.72 (d, J=3.1 Hz, 1H), 5.60 (s, 2H), 5.24-5.15 (m, 1H), 1.35 (d, J=7.0 Hz, 3H).

### EXAMPLE 208

### 4-((1-((4′-fluoro-[1,1′-biphenyl]-4-yl)methyl)-5-phenyl-1H-indole-7-carboxamido)methyl)benzoic acid

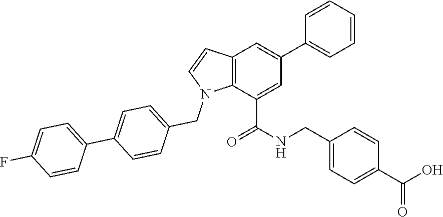 [SMILES: O=C(O)c1ccc(CNC(=O)c2cc(-c3ccccc3)cc3ccn(Cc4ccc(-c5ccc(F)cc5)cc4)c23)cc1]

Starting with 4-(bromomethyl)-4′-fluoro-1,1′-biphenyl, and phenylboronic acid, the title product was obtained as a white solid by following the same procedures as described in Example 75. LCMS (ESI): 555.2 [M+1]⁺.

¹HNMR (400 MHz, DMSO-d₆) δ 9.06 (t, J=6.0 Hz, 1H), 8.02 (s, 1H), 7.84 (d, J=8.0 Hz, 2H), 7.75 (d, J=7.7 Hz, 2H), 7.67-7.59 (m, 3H), 7.57 (d, J=1.8 Hz, 1H), 7.52-7.42 (m, 4H), 7.36 (dd, J=7.7, 4.4 Hz, 3H), 7.26 (t, J=8.7 Hz, 2H), 6.95 (d, J=8.1 Hz, 2H), 6.73 (d, J=3.1 Hz, 1H), 5.66 (s, 2H), 4.52 (d, J=5.8 Hz, 2H).

### EXAMPLE 209

### 4-((1-((4′-fluoro-[1,1′-biphenyl]-4-yl)methyl)-5-(4-fluorophenyl)-1H-indole-7-carboxamido)methyl)benzoic acid

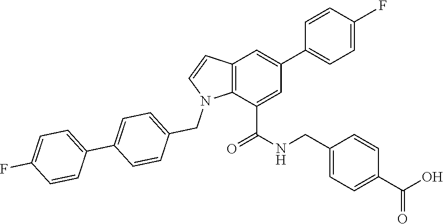 [SMILES: O=C(O)c1ccc(CNC(=O)c2cc(-c3ccc(F)cc3)cc3ccn(Cc4ccc(-c5ccc(F)cc5)cc4)c23)cc1]

Starting with 4-(bromomethyl)-4′-fluoro-1,1′-biphenyl, and (4-fluorophenyl)boronic acid, the title product was obtained as a white solid (28.8 mg) by following the same procedures as described in Example 75. LCMS (ESI): 573.2 [M+1]⁺.

¹HNMR (400 MHz, DMSO-d₆) δ 9.05 (t, J=6.0 Hz, 1H), 8.00 (s, 1H), 7.84 (d, J=8.0 Hz, 2H), 7.77 (dd, J=8.5, 5.5 Hz, 2H), 7.67-7.59 (m, 3H), 7.53 (s, 1H), 7.45 (d, J=8.0 Hz, 2H), 7.36 (d, J=8.0 Hz, 2H), 7.33-7.24 (m, 4H), 6.95 (d, J=8.0 Hz, 2H), 6.72 (d, J=3.1 Hz, 1H), 5.66 (s, 2H), 4.52 (d, J=5.8 Hz, 2H).

### EXAMPLE 210

### 4-((5-(3,5-difluorophenyl)-1-((4′-fluoro-[1,1′-biphenyl]-4-yl)methyl)-1H-indole-7-carboxamido)methyl)benzoic acid

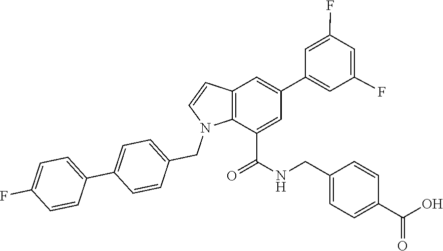 [SMILES: O=C(O)c1ccc(CNC(=O)c2cc(-c3cc(F)cc(F)c3)cc3ccn(Cc4ccc(-c5ccc(F)cc5)cc4)c23)cc1]

Starting with 4-(bromomethyl)-4′-fluoro-1,1′-biphenyl, and (3,5-difluorophenyl)boronic acid, the title product was obtained as a white solid by following the same procedures as described in Example 75. LCMS (ESI): 591.2 [M+1]⁺.

¹H NMR (400 MHz, DMSO-d₆) δ 9.03 (t, J=6.0 Hz, 1H), 8.13 (s, 1H), 7.83 (d, J=7.9 Hz, 2H), 7.67 (d, J=3.2 Hz, 1H), 7.62 (q, J=5.3 Hz, 3H), 7.51 (d, J=8.1 Hz, 2H), 7.44 (d, J=7.9 Hz, 2H), 7.35 (d, J=8.0 Hz, 2H), 7.25 (t, J=8.7 Hz, 2H), 7.18 (t, J=9.3 Hz, 1H), 6.93 (d, J=8.0 Hz, 2H), 6.75 (d, J=3.1 Hz, 1H), 5.68 (s, 2H), 4.53 (d, J=5.9 Hz, 2H).

### EXAMPLE 211

### 4-((5-(3,4-difluorophenyl)-1-((4′-fluoro-[1,1′-biphenyl]-4-yl)methyl)-1H-indole-7-carboxamido)methyl)benzoic acid

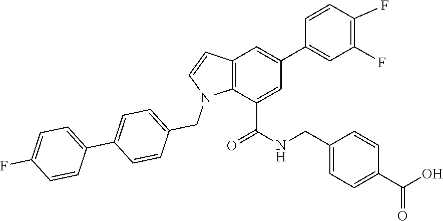 [SMILES: O=C(O)c1ccc(CNC(=O)c2cc(-c3ccc(F)c(F)c3)cc3ccn(Cc4ccc(-c5ccc(F)cc5)cc4)c23)cc1]

Starting with 4-(bromomethyl)-4′-fluoro-1,1′-biphenyl, an (3,4-difluorophenyl)boronic acid, the title product was obtained as a white solid by following the same procedures as described in Example 75. LCMS (ESI): 591.2 [M+1]⁺.

¹HNMR (400 MHz, DMSO-d₆) δ 12.81 (s, 1H), 8.96 (t, J=5.9 Hz, 1H), 7.99 (d, J=1.8 Hz, 1H), 7.82-7.72 (m, 3H), 7.60 (d, J=3.2 Hz, 1H), 7.60-7.41 (m, 5H), 7.38 (d, J=7.9 Hz, 2H), 7.29 (d, J=7.9 Hz, 2H), 7.19 (t, J=8.8 Hz, 2H), 6.87 (d, J=7.9 Hz, 2H), 6.66 (d, J=3.1 Hz, 1H), 5.61 (s, 2H), 4.46 (d, J=5.8 Hz, 2H).

### EXAMPLE 212

(S)-4-(1-(1-((4′-fluoro-[1,1′-biphenyl]-4-yl)methyl)-5-phenyl-1H-indole-7-carboxamido)ethyl)benzoic acid

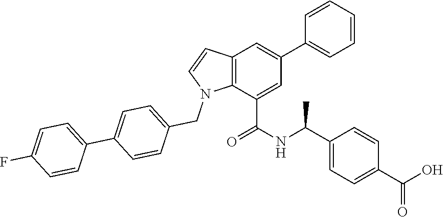 [SMILES: C[C@H](NC(=O)c1cc(-c2ccccc2)cc2ccn(Cc3ccc(-c4ccc(F)cc4)cc3)c12)c1ccc(C(=O)O)cc1]

Starting with 4-(bromomethyl)-4′-fluoro-1,1′-biphenyl, methyl (S)-4-(1-aminoethyl)benzoate and phenylboronic acid, the title product was obtained as a white solid by following the same procedures as described in Example 75. LCMS (ESI): 569.2 [M+1]⁺.

¹HNMR (400 MHz, DMSO-d₆) δ 12.85 (s, 1H), 9.00 (d, J=7.9 Hz, 1H), 8.01 (d, J=1.8 Hz, 1H), 7.93-7.86 (m, 2H), 7.76 (d, J=7.6 Hz, 2H), 7.67-7.56 (m, 3H), 7.54-7.46 (m, 5H), 7.38 (dd, J=13.5, 7.6 Hz, 3H), 7.26 (t, J=8.8 Hz, 2H), 6.94 (d, J=8.0 Hz, 2H), 6.72 (d, J=3.1 Hz, 1H), 5.59 (s, 2H), 5.23-5.19 (m, 1H), 1.35 (d, J=7.0 Hz, 3H).

### EXAMPLE 213

### (S)-4-(1-(1-((4′-fluoro-[1,1′-biphenyl]-4-yl)methyl)-5-(4-fluorophenyl)-1H-indole-7-carboxamido)ethyl)benzoic acid

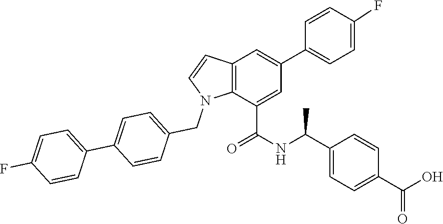 [SMILES: C[C@H](NC(=O)c1cc(-c2ccc(F)cc2)cc2ccn(Cc3ccc(-c4ccc(F)cc4)cc3)c12)c1ccc(C(=O)O)cc1]

Starting with 4-(bromomethyl)-4′-fluoro-1,1′-biphenyl, methyl (S)-4-(1-aminoethyl)benzoate and (4-fluorophenyl)boronic acid, the title product was obtained as a white solid by following the same procedures as described in Example 75. LCMS (ESI): 587.2 [M+1]⁺.

¹HNMR (400 MHz, DMSO-d₆) δ 12.73 (s, 1H), 8.98 (d, J=7.9 Hz, 1H), 7.99 (d, J=1.8 Hz, 1H), 7.89 (d, J=8.4 Hz, 2H), 7.82-7.74 (m, 2H), 7.65 (d, J=3.2 Hz, 1H), 7.63-7.56 (m, 2H), 7.52 (s, 1H), 7.51-7.46 (m, 2H), 7.40 (d, J=8.0 Hz, 2H), 7.33 (t, J=8.8 Hz, 2H), 7.26 (t, J=8.8 Hz, 2H), 6.93 (d, J=8.0 Hz, 2H), 6.71 (d, J=3.1 Hz, 1H), 5.59 (s, 2H), 5.20 (p, J=7.4 Hz, 1H), 1.35 (d, J=7.0 Hz, 3H).

### EXAMPLE 214

### (S)-4-(1-(5-(3,5-difluorophenyl)-1-((4′-fluoro-[1,1′-biphenyl]-4-yl)methyl)-1H-indole-7-carboxamido)ethyl)benzoic acid

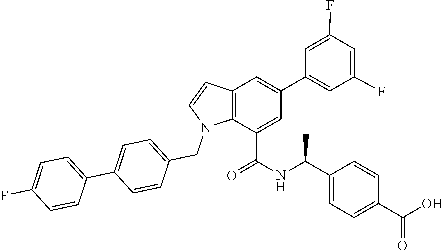 [SMILES: C[C@H](NC(=O)c1cc(-c2cc(F)cc(F)c2)cc2ccn(Cc3ccc(-c4ccc(F)cc4)cc3)c12)c1ccc(C(=O)O)cc1]

Starting with 4-(bromomethyl)-4′-fluoro-1,1′-biphenyl, methyl (S)-4-(1-aminoethyl)benzoate and (3,5-difluorophenyl)boronic acid, the title product was obtained as a white solid by following the same procedures as described in Example 75. LCMS (ESI): 605.2 [M+1]⁺.

¹HNMR (400 MHz, DMSO-d₆) δ 12.83 (s, 1H), 8.95 (d, J=7.8 Hz, 1H), 8.12 (d, J=1.8 Hz, 1H), 7.89 (d, J=7.9 Hz, 2H), 7.67 (d, J=3.2 Hz, 1H), 7.63-7.48 (m, 7H), 7.39 (d, J=7.9 Hz, 2H), 7.30-7.16 (m, 3H), 6.92 (d, J=7.9 Hz, 2H), 6.73 (d, J=3.2 Hz, 1H), 5.68-5.53 (m, 2H), 5.23-5.17 (m, 1H), 1.37 (d, J=7.0 Hz, 3H).

### EXAMPLE 215

### (S)-4-(1-(5-(3,4-difluorophenyl)-1-((4′-fluoro-[1,1′-biphenyl]-4-yl)methyl)-1H-indole-7-carboxamido)ethyl)benzoic acid

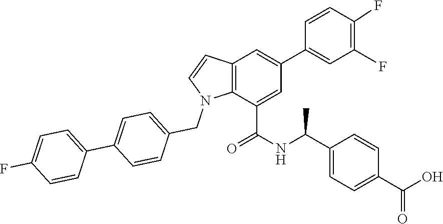 [SMILES: C[C@H](NC(=O)c1cc(-c2ccc(F)c(F)c2)cc2ccn(Cc3ccc(-c4ccc(F)cc4)cc3)c12)c1ccc(C(=O)O)cc1]

Starting with 4-(bromomethyl)-4′-fluoro-1,1′-biphenyl, methyl (S)-4-(1-aminoethyl)benzoate and (3,4-difluorophenyl)boronic acid, the title product was obtained as a white solid by following the same procedures as described in Example 75. LCMS (ESI): 605.2 [M+1]⁺.

¹HNMR (400 MHz, DMSO-d₆) δ 12.82 (s, 1H), 8.95 (d, J=7.9 Hz, 1H), 8.04 (s, 1H), 7.94-7.78 (m, 3H), 7.66 (d, J=3.2 Hz, 1H), 7.65-7.54 (m, 3H), 7.55-7.47 (m, 4H), 7.40 (d, J=8.0 Hz, 2H), 7.26 (t, J=8.8 Hz, 2H), 6.92 (d, J=8.0 Hz, 2H), 6.71 (d, J=3.1 Hz, 1H), 5.73-5.51 (m, 2H), 5.23-5.17 (m, 1H), 1.36 (d, J=7.0 Hz, 3H).

### EXAMPLE 216

### 4-((5-(3,4-difluorophenyl)-1-(naphthalen-2-ylmethyl)-1H-indole-7-carboxamido)methyl)benzoic acid

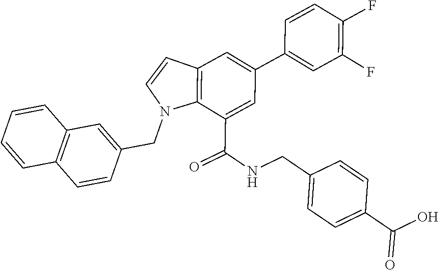 [SMILES: O=C(O)c1ccc(CNC(=O)c2cc(-c3ccc(F)c(F)c3)cc3ccn(Cc4ccc5ccccc5c4)c23)cc1]

Starting with 2-(bromomethyl)naphthalene and (3,4-difluorophenyl)boronic acid, the title product was obtained as a white solid by following the same procedures as described in Example 75. LCMS (ESI): 547.5[M+1]⁺.

¹HNMR (400 MHz, DMSO-d₆) δ 12.80 (s, 1H), 8.92 (t, J=5.9 Hz, 1H), 8.03 (s, 1H), 7.85-7.81 (m, 1H), 7.80-7.68 (m, 6H), 7.59-7.52 (m, 1H), 7.51-7.48 (m, 2H), 7.47-7.42 (m, 2H), 7.38 (s, 1H), 7.21 (d, J=8.0 Hz, 2H), 7.10-7.03 (m, 1H), 6.73 (d, J=3.1 Hz, 1H), 5.79 (s, 2H), 4.44 (d, J=5.7 Hz, 2H).

### EXAMPLE 217

### (S)-4-(1-(5-(3,4-difluorophenyl)-1-(naphthalen-2-ylmethyl)-1H-indole-7-carboxamido)ethyl)benzoic acid

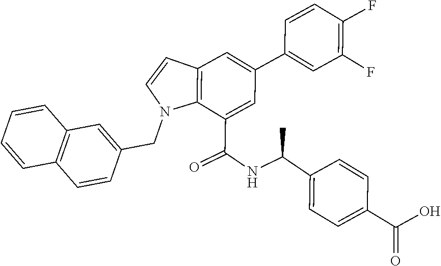 [SMILES: C[C@H](NC(=O)c1cc(-c2ccc(F)c(F)c2)cc2ccn(Cc3ccc4ccccc4c3)c12)c1ccc(C(=O)O)cc1]

Starting with 2-(bromomethyl)naphthalene, methyl (S)-4-(1-aminoethyl)benzoate and (3,4-difluorophenyl) boronic acid, the title product was obtained as a white solid by following the same procedures as described in Example 75. LCMS (ESI): 561.5[M+1]⁺.

¹HNMR (400 MHz, DMSO-d₆) δ 8.84 (d, J=7.6 Hz, 1H), 8.02 (s, 1H), 7.85-7.76 (m, 4H), 7.75-7.69 (m, 2H), 7.68 (d, J=3.2 Hz, 1H), 7.60-7.49 (m, 2H), 7.47-7.42 (m, 3H), 7.40 (d, J=8.0 Hz, 2H), 7.36 (s, 1H), 7.06-7.01 (m, 1H), 6.72 (d, J=3.1 Hz, 1H), 5.80-5.64 (m, 2H), 5.12-5.04 (m, 1H), 1.11 (d, J=7.0 Hz, 3H).

### EXAMPLE 218

### 4-((5-(3,4-difluorophenyl)-1-(naphthalen-2-ylmethyl)-1H-indazole-7-carboxamido)methyl)benzoic acid

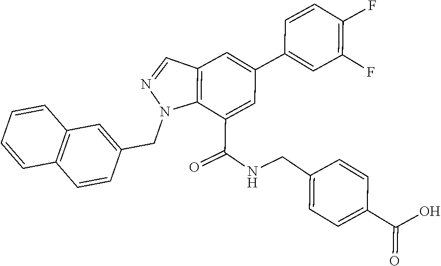 [SMILES: O=C(O)c1ccc(CNC(=O)c2cc(-c3ccc(F)c(F)c3)cc3cnn(Cc4ccc5ccccc5c4)c23)cc1]

Starting with methyl 4-(aminomethyl)benzoate and (3,4-difluorophenyl)boronic acid, the title product was obtained as a white solid by following the same procedures as described in Example 167. LCMS (ESI): 548.5 [M+1]⁺.

¹HNMR (400 MHz, DMSO-d₆) δ 12.81 (s, 1H), 9.20 (t, J=5.9 Hz, 1H), 8.37 (s, 1H), 8.26 (d, J=1.7 Hz, 1H), 7.91-7.81 (m, 3H), 7.76-7.69 (m, 4H), 7.65-7.59 (m, 1H), 7.58-7.49 (m, 1H), 7.48-7.42 (m, 3H), 7.28 (d, J=8.0 Hz, 2H), 7.13-7.07 (m, 1H), 6.02 (s, 2H), 4.54 (d, J=5.7 Hz, 2H).

### EXAMPLE 219

### (S)-4-(1-(5-(3,4-difluorophenyl)-1-(naphthalen-2-ylmethyl)-1H-indazole-7-carboxamido)ethyl)benzoic acid

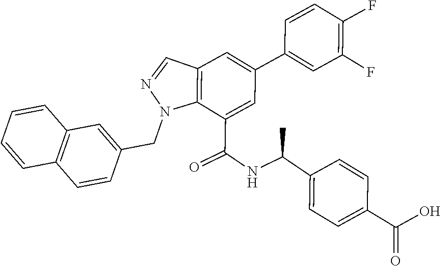 [SMILES: C[C@H](NC(=O)c1cc(-c2ccc(F)c(F)c2)cc2cnn(Cc3ccc4ccccc4c3)c12)c1ccc(C(=O)O)cc1]

Starting with (3,4-difluorophenyl)boronic acid, the title product was obtained as a white solid by following the same procedures described in Example 167. LCMS (ESI): 562.4 [M+1]⁺.

¹HNMR (400 MHz, DMSO-d₆) δ 12.83 (s, 1H), 9.09 (d, J=7.6 Hz, 1H), 8.38 (s, 1H), 8.27 (d, J=1.7 Hz, 1H), 7.93-7.81 (m, 4H), 7.80-7.71 (m, 3H), 7.68-7.54 (m, 2H), 7.50-7.43 (m, 5H), 7.09 (dd, J=8.5, 1.8 Hz, 1H), 5.97 (s, 2H), 5.21 (p, J=7.1 Hz, 1H), 1.26 (d, J=6.8 Hz, 3H).

### EXAMPLE 220

(S)-4-(1-(5-(3,4-difluorophenyl)-1-(3-(trifluoromethyl)benzyl)-1H-indazole-7-carboxamido)ethyl)benzoic acid

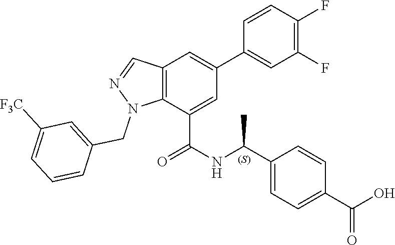

Starting with (3,4-difluorophenyl)boronic acid, the title product was obtained as a white solid by following the same procedures as described in Example 134. LCMS (ESI): 580 [M+1]⁺.

¹HNMR (400 MHz, DMSO-d₆) δ 12.84 (s, 1H), 9.22 (d, J=7.6 Hz, 1H), 8.38 (s, 1H), 8.28 (d, J=1.7 Hz, 1H), 8.00-7.86 (m, 3H), 7.69-7.54 (m, 3H), 7.51-7.40 (m, 3H), 7.33 (s, 1H), 7.13 (d, J=7.7 Hz, 1H), 5.86 (s, 2H), 5.21-5.10 (m, 1H), 1.39 (d, J=7.0 Hz, 3H).

### EXAMPLE 221

### (1r,4r)-4-((5-phenyl-1-(3-(trifluoromethyl)benzyl)-1H-indazole-7-carboxamido)methyl)cyclohexane-1-carboxylic acid

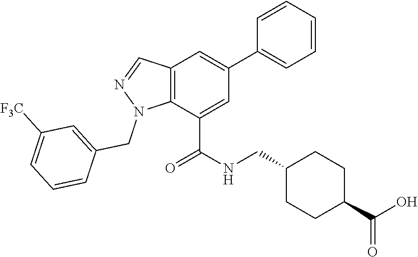 [SMILES: O=C(NC[C@H]1CC[C@H](C(=O)O)CC1)c1cc(-c2ccccc2)cc2cnn(Cc3cccc(C(F)(F)F)c3)c12]

### Step 1 methyl 5-phenyl-1H-indazole-7-carboxylate

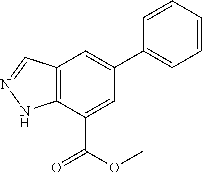 [SMILES: COC(=O)c1cc(-c2ccccc2)cc2cn[nH]c12]

A mixture of methyl 5-bromo-1H-indazole-7-carboxylate (0.8 g), phenylboronic acid (4.0 g), 1,1′-bis(diphenylphosphino)ferrocene-palladium(II)dichloride dichloromethane complex (0.27 g), potassium acetate (4.8 g) in 1,4-dioxane/water (80 mL/8 mL) was stirred for 2 h at 70° C. under nitrogen. The reaction mixture was concentrated and purified by silica gel column chromatography (petroleum ether: ethyl acetate: =5:1) to afford the title compound as a yellow solid (0.78 g). LCMS (ESI): 253.22 [M+1]⁺.

### Step 2 methyl 5-phenyl-1-(3-(trifluoromethyl)benzyl)-1H-indazole-7-carboxylate

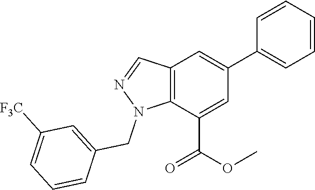 [SMILES: COC(=O)c1cc(-c2ccccc2)cc2cnn(Cc3cccc(C(F)(F)F)c3)c12]

A mixture of the product of Step 1 (0.77 g) and cesium carbonate (3.0 g) in anhydrous N,N-dimethylformamide (30 mL) was stirred at r.t. for 1 h, and then treated with 1-(bromomethyl)-3-(trifluoromethyl)benzene (1.1 g). After stirring at r.t. for 2 h, the reaction mixture was concentrated and purified by silica gel column chromatography (petroleum ether: ethyl acetate: =10:1) to afford the title compound as a yellow solid (1.0 g). LCMS (ESI): 411.32 [M+1]⁺.

### Step 3 5-phenyl-1-(3-(trifluoromethyl)benzyl)-1H-indazole-7-carboxylic acid

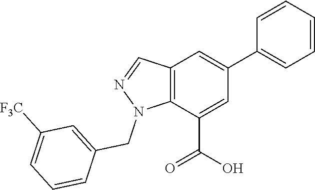 [SMILES: O=C(O)c1cc(-c2ccccc2)cc2cnn(Cc3cccc(C(F)(F)F)c3)c12]

To a solution of the product of Step 2 (1.0 g) in acetonitrile/water (30 mL/15 mL) was added lithium hydroxide monohydrate (2.1 g) at r.t. The reaction mixture was stirred at 80° C. for 1 h. After being cooled to 0° C., the reaction mixture was treated with 6 N HCl to pH≈6. The reaction mixture was extracted 3 x 50 mL ethyl acetate. The combined organic layer was washed with 200 mL of brine, dried over anhydrous sodium sulfate, and concentrated to afford the title compound as a white solid (0.95 g). LCMS (ESI): 397.15 [M+1]⁺.

### Step 4 methyl (1r,4r)-4-((5-phenyl-1-(3-(trifluoromethyl)benzyl)-1H-indazole-7-carboxamido)methyl)cyclohexane-1-carboxylate

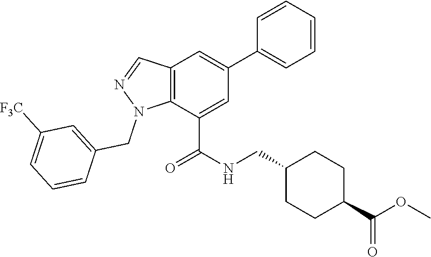 [SMILES: COC(=O)[C@H]1CC[C@H](CNC(=O)c2cc(-c3ccccc3)cc3cnn(Cc4cccc(C(F)(F)F)c4)c23)CC1]

To a mixture of the product of Step 3 (50 mg), methyl (1r,4r)-4-(aminomethyl)cyclohexane-1-carboxylate hydrochloride (31 mg) and DIPEA (49 mg) in DMF (10 mL) at 0° C. was added HATU (72 mg). After stirring at r.t. for 1 h, 5 mL water was added and stirred at r.t. for 1 h, the solid was collected by filtration, washed with water, and air-dried to afford the title compound as a white solid (60 mg). LCMS (ESI): 550.22 [M+1]⁺.

### Step 5 (1r,4r)-4-((5-phenyl-1-(3-(trifluoromethyl)benzyl)-1H-indazole-7-carboxamido)methyl)cyclohexane-1-carboxylic acid

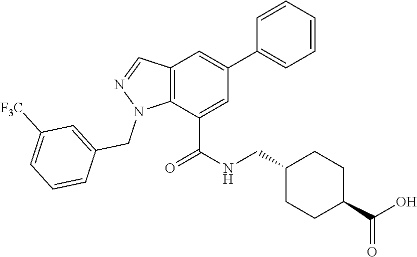 [SMILES: O=C(NC[C@H]1CC[C@H](C(=O)O)CC1)c1cc(-c2ccccc2)cc2cnn(Cc3cccc(C(F)(F)F)c3)c12]

To a solution of the product of Step 4 (60 mg) in acetonitrile/water (10 mL/5 mL) was added lithium hydroxide monohydrate (106 mg). The reaction mixture was stirred at 70° C. for 1 h. After being cooled to r.t., the mixture was treated with 6 N hydrochloric acid to pH≈6. The solid was collected by filtration, washed with water, and air-dried to afford the title compound as a white solid (52 mg). LCMS (ESI): 536.32 [M+1]⁺.

¹H NMR (400 MHz, DMSO-d₆) δ 11.99 (s, 1H), 8.75-8.68 (m, 1H), 8.37 (s, 1H), 8.26-8.20 (m, 1H), 7.82-7.76 (m, 3H), 7.61 (d, J=7.8 Hz, 1H), 7.55-7.47 (m, 3H), 7.44-7.36 (m, 2H), 7.22 (d, J=7.7 Hz, 1H), 5.98 (s, 2H), 3.10 (t, J=6.2 Hz, 2H), 2.13-2.07 (m, 1H), 1.90-1.80 (m, 2H), 1.74-1.65 (m, 2H), 1.45-1.35 (m, 1H), 1.24-1.14 (m, 2H), 0.98-0.84 (m, 2H).

### EXAMPLE 222

### (±) (1r,4r)-4-1-(5-phenyl-1-(3-(trifluoromethyl)benzyl)-1H-indazole-7-carboxamido)ethyl)cyclohexane-1-carboxylic acid

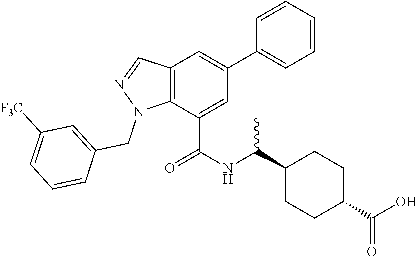 [SMILES: CC(NC(=O)c1cc(-c2ccccc2)cc2cnn(Cc3cccc(C(F)(F)F)c3)c12)[C@H]1CC[C@H](C(=O)O)CC1]

### Step 1 methyl (1r, 4r)-4-(methoxy(methyl)carbamoyl)cyclohexane-1-carboxylate

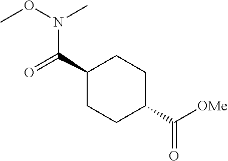 [SMILES: COC(=O)[C@H]1CC[C@H](C(=O)N(C)OC)CC1]

A 40 mL screw-top vial was charged with (1r, 4r)-4-(methoxycarbonyl)cyclohexane-1-carboxylic acid (1.86 g), 1,1′-carbonyldiimidazole(2.1 g) in dichloromethane (50 ml) and stirred 1 h. N,O-dimethyl hydroxylamine hydrochloride(1.94 g) was added and stirred at r.t. for 12 h. Water (100 mL) was added.

The reaction mixture was extracted with dichloromethane (3×50 ml). The organic layer was washed with sat. brine, dried over anhydrous sodium sulfate, filtered and concentrated. The residue was purified by silica gel column chromatography (petroleum ether: ethyl acetate: 0-30%) to afford a product as oil 2.3 g.

### LCMS (ESI): 230.4 [M+1]⁺.

### Step 2 methyl (1r, 4r)-4-acetylcyclohexane-1-carboxylate

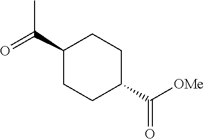

To a mixture of the product of Step 1 (2.3 g) in tetrahydrofuran (40 ml) cooled at 0° C. was added methyl magnesium bromide (15 ml, 1M in THF). The reaction mixture was stirred at r.t. for 12 h. Ammonium chloride (aq) (100 mL) was added. The reaction mixture was extracted with ethyl acetate (3×50). The organic layer was washed with sat. brine, dried over anhydrous sodium sulfate, filtered and concentrated.

The residue was purified by silica gel column chromatography (petroleum ether: ethyl acetate: 0-15%) to afford a product as oil 1.7 g. LCMS (ES+): 185.2 [M+1]⁺.

### Step 3 (±)methyl (1r, 4r)-4-(-1-aminoethyl)cyclohexane-1-carboxylate hydrochloride

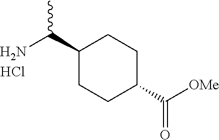

A mixture of the product of Step 2 (368 mg, 1.0 eq.), ammonium formate (630 mg), and palladium (37 mg) in 10 ml methanol was stirred at 70° C. for 12 h. The reaction mixture was directly concentrated in vacuo, poured in 100 mL water, extracted with ethyl acetate (3×50). The combined extracts were dried over anhydrous sodium sulfate, filtered, then treated with 1 mL hydrochloric acid (4M in 1,4-dioxane). The mixture was concentrated in vacuo to afford crude product. LCMS (ES+): 186.3 [M+1]⁺.

### Step 4 (±)methyl (1r,4r)-4-(1-(5-phenyl-1-(3-(trifluoromethyl)benzyl)-1H-indazole-7-carboxamido)ethyl)cyclohexane-1-carboxylate

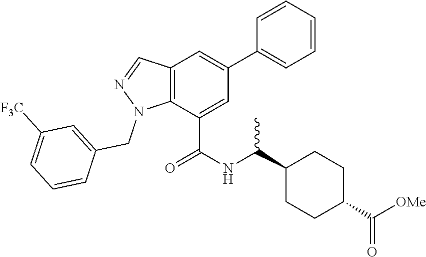 [SMILES: COC(=O)[C@H]1CC[C@H](C(C)NC(=O)c2cc(-c3ccccc3)cc3cnn(Cc4cccc(C(F)(F)F)c4)c23)CC1]

A mixture of 5-phenyl-1-(3-(trifluoromethyl)benzyl)-1H-indazole-7-carboxylic acid (40 mg, 0.1 mmol), 1.2 eq the product of Step 3, 0-(7-azabenzotriazol-1-yl)-N,N,N′,N′-tetramethyl uronium hexafluorophosphate (114 mg), and DIPEA (39 mg) in anhydrous DMF (10 mL) was stirred 12 h at r.t.

Water (100 mL) was added and the mixture was extracted with 3×50 mL ethyl acetate. The combined extracts were washed with brine, dried over anhydrous sodium sulfate, filtered. The filtrate was concentrated and the residue was purified by silica gel column chromatography (petroleum ether ethyl acetate: 0-40%) to afford the title product as an oil (55 mg). LCMS (ES+): 564.6 [M+1]⁺.

### Step 5 (±) (1r,4r)-4-(1-(5-phenyl-1-(3-(trifluoromethyl)benzyl)-1H-indazole-7-carboxamido)ethyl)cyclohexane-1-carboxylic acid

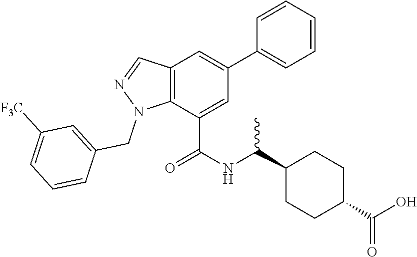 [SMILES: CC(NC(=O)c1cc(-c2ccccc2)cc2cnn(Cc3cccc(C(F)(F)F)c3)c12)[C@H]1CC[C@H](C(=O)O)CC1]

The product of Step 4 (55 mg,) and lithium hydroxide monohydrate (42 mg) were dissolved in tetrahydrofuran/water (6 mL/4 mL). After stirring for 12 h at 40° C., the reaction mixture was acidified with 2 mL 6M hydrochloric acid, and tetrahydrofuran was removed in vacuo. The reaction mixture was then extracted with 3×50 mL ethyl acetate. The combined extracts were washed with brine, dried over anhydrous sodium sulfate, filtered, and concentrated to afford the title product (50 mg). LCMS (ES+): 549.6 [M+1]⁺.

### EXAMPLE 223 AND EXAMPLE 224

### (1S,4r)-4-((S)-1-(5-phenyl-1-(3-(trifluoromethyl)benzyl)-1H-indazole-7-carboxamido)ethyl)cyclohexane-1-carboxylic acid

### (1R,4r)-4-((R)-1-(5-phenyl-1-(3-(trifluoromethyl)benzyl)-1H-indazole-7-carboxamido)ethyl)cyclohexane-1-carboxylic acid

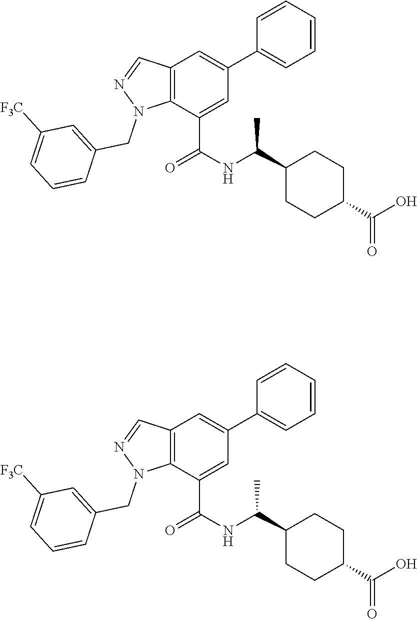 [SMILES: C[C@H](NC(=O)c1cc(-c2ccccc2)cc2cnn(Cc3cccc(C(F)(F)F)c3)c12)[C@H]1CC[C@H](C(=O)O)CC1 ; C[C@@H](NC(=O)c1cc(-c2ccccc2)cc2cnn(Cc3cccc(C(F)(F)F)c3)c12)[C@H]1CC[C@H](C(=O)O)CC1]

### Example 222 was resolved by the following conditions to provide EXAMPLE 223 AND EXAMPLE 224

Column type: CHIRALPAK IB N-5,5 m, 250 mm×4.6 mm
Mobile phase: n-hexane/EtOH (70:30)+0.2% TFA
Flow rate: 1 mL/min.

### Example 223: retention time at 5.188 min

### Example 224: retention time at 8.485 min

1-15. (canceled)

16. A compound of Formula I or a pharmaceutically acceptable salt thereof: 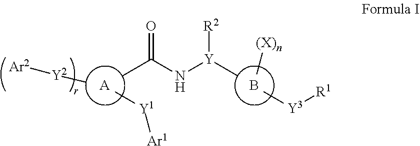
wherein:
R¹ represents —CO₂H, —C(O)NHS(O)₂R⁷, —NHC(O)NHSO₂R⁷, -1H-tetrazolyl, —P(O)(OH)₂, -1,2,4-oxadiazol-5(4H) -one or -tetrazol-5(4H)-one;
Y is N, CR³ or 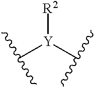
is a bond; when Y is CR³, 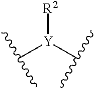
includes both R and S configurations;
R², R³ are independently selected from the group consisting of: hydrogen, C₁₋₆alkyl, C₃₋₆cyclolkyl, C₁₋₆fluorocycloalkyl, C₁₋₆fluoroalkyl, or R², R³, together with the carbon atom to which they are both attached to, complete a three- to six-membered carbocyclic ring which is optionally substituted with R^{c} or a three- to six-membered ring which contains one or two heteroatom(s) such as S, O or NR^{b}, wherein R^{b} is selected from the group consisting of hydrogen, C₁₋₆alkyl, C₃₋₆cyclolkyl, C₃₋₆fluorocycloalkyl, C₁₋₆fluoroalkyl, C₀₋₆alkylene-aryl, C₀₋₆alkylene-heteroaryl, C(O) C₁₋₆alkyl, C(O)aryl, S(0)₂C₁₋₆alkyl, S(0)₂C₃₋₆cycloalkyl or S(O)₂aryl;
X is absent or is independently halogen, —CN, C₁₋₆alkyl, OC₁₋₆alkyl, C₁₋₆haloalkyl C₃₋₆cycloalkyl or OC₁₋₆haloalkyl; 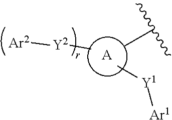
is selected from the following structures: 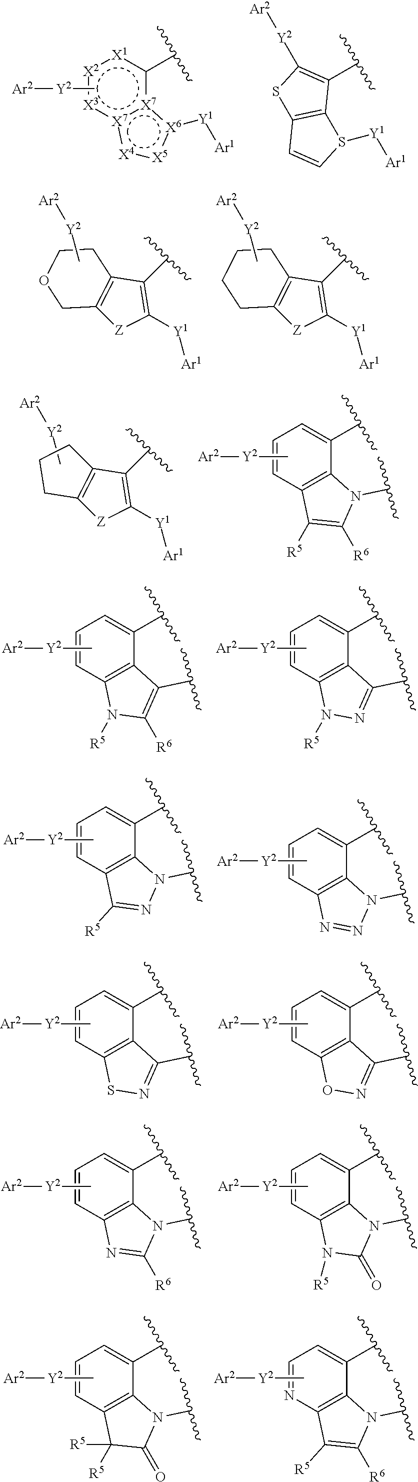 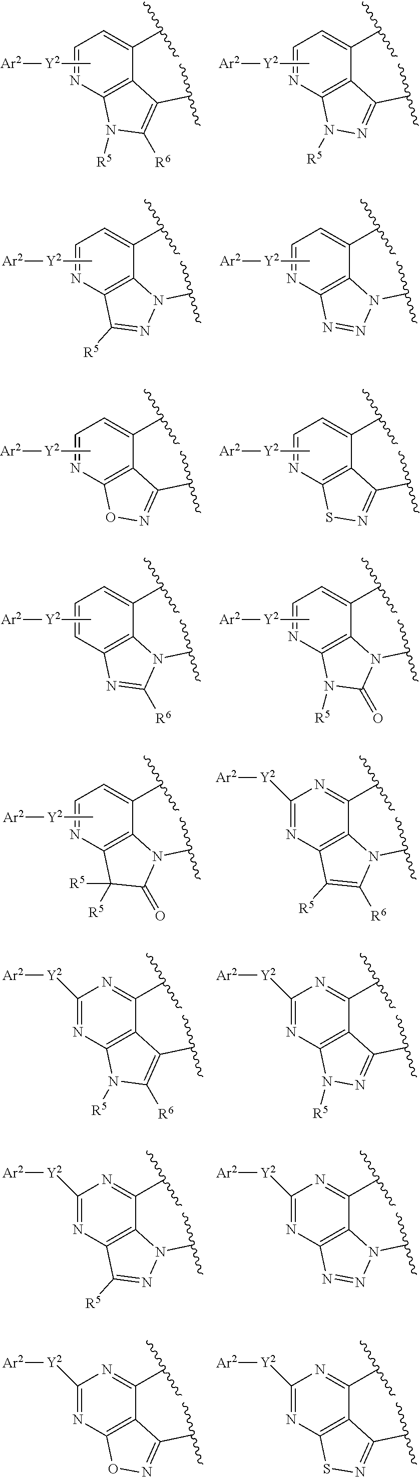 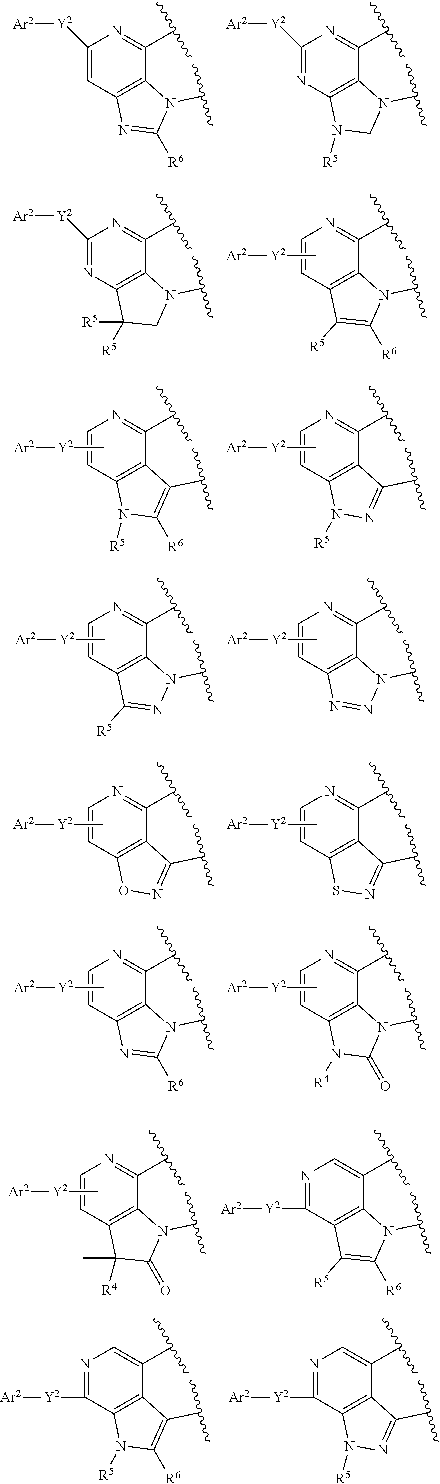 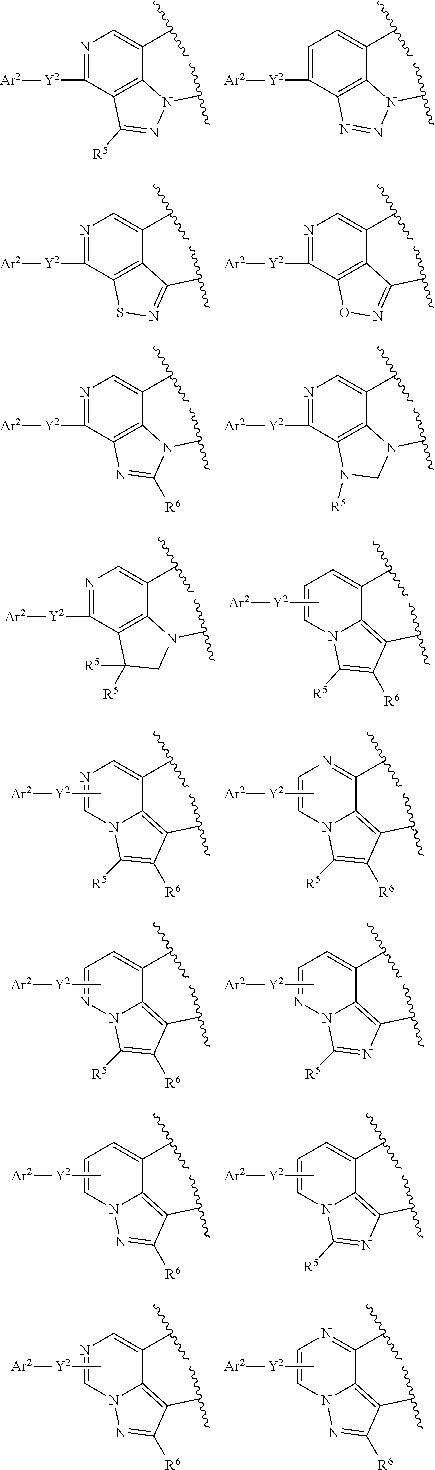 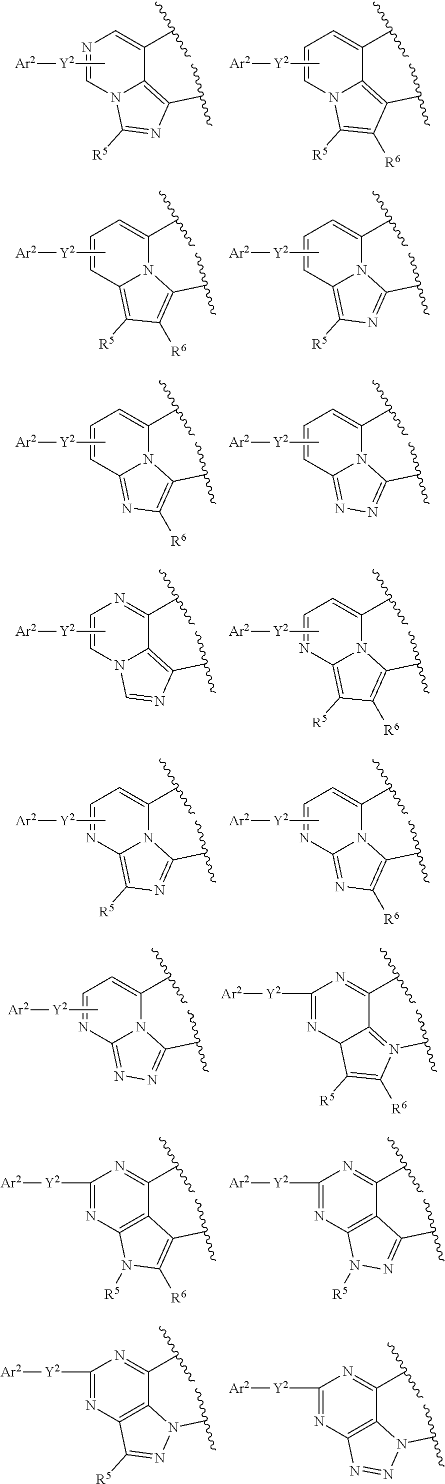 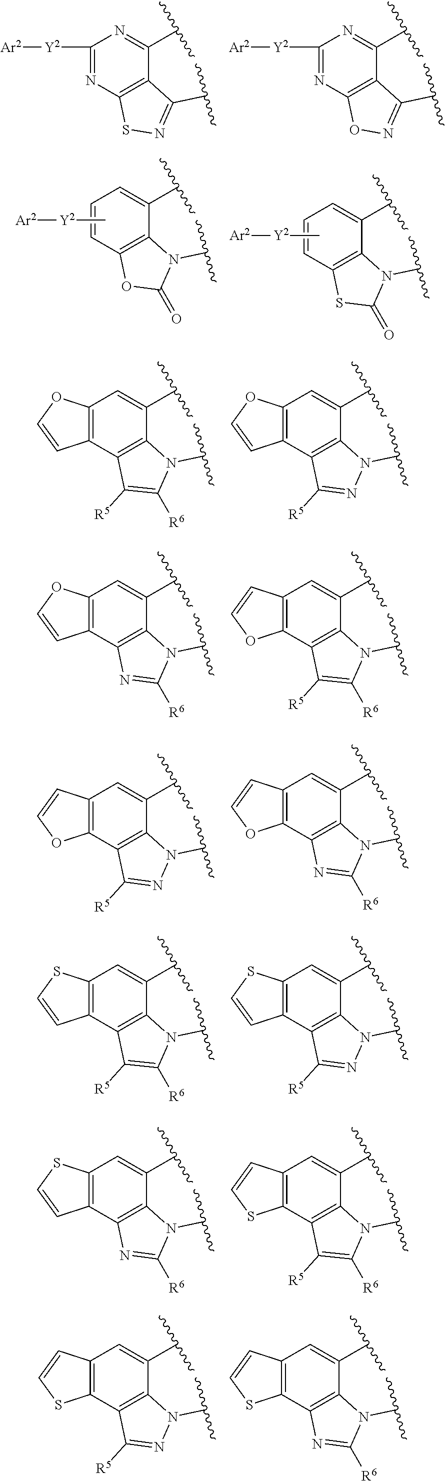 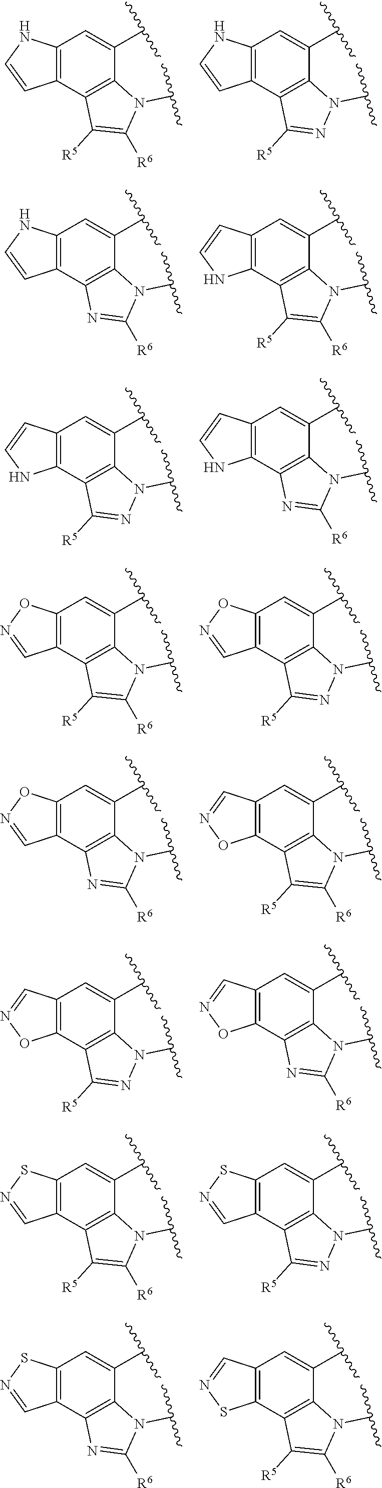 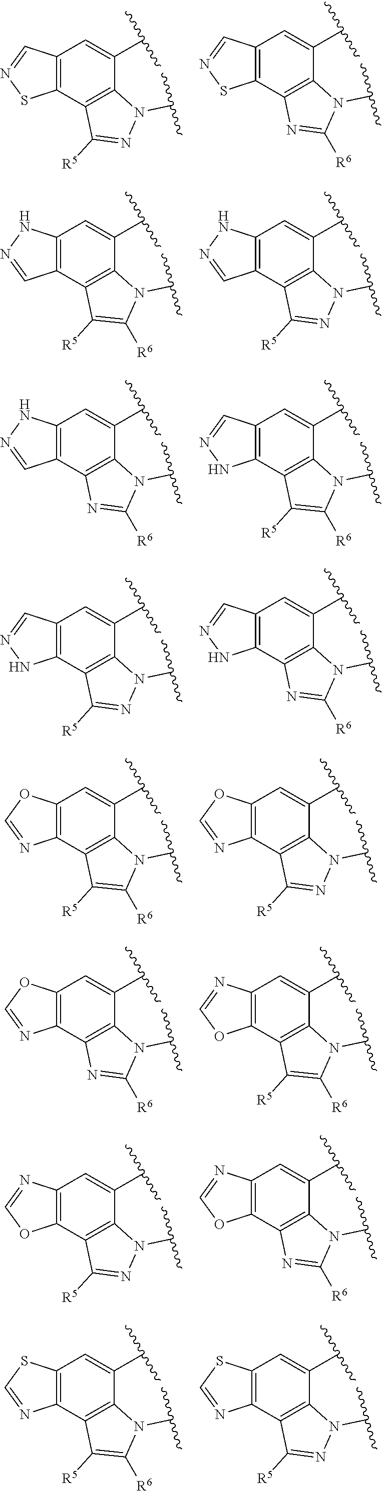 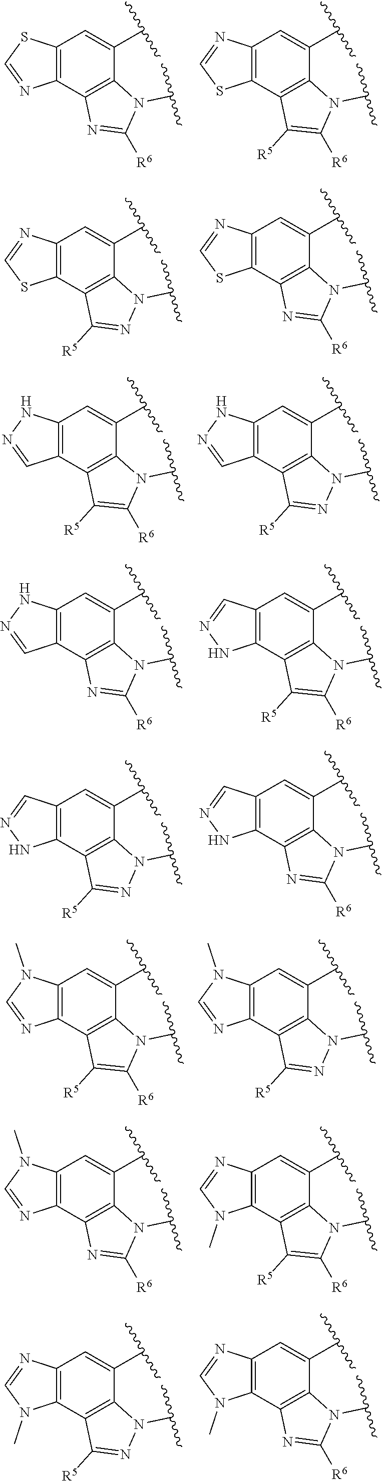 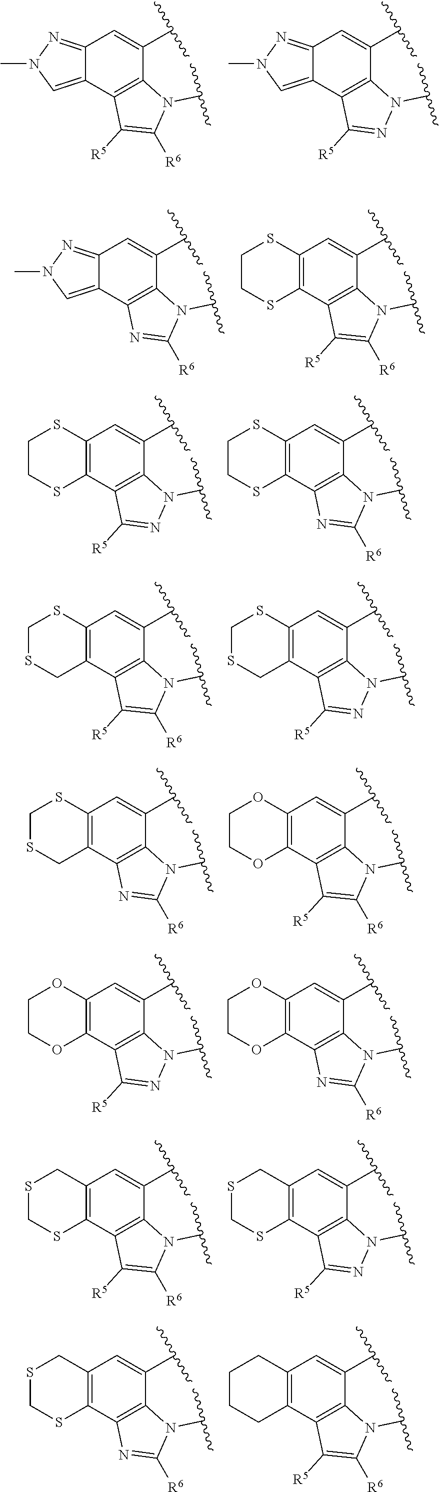 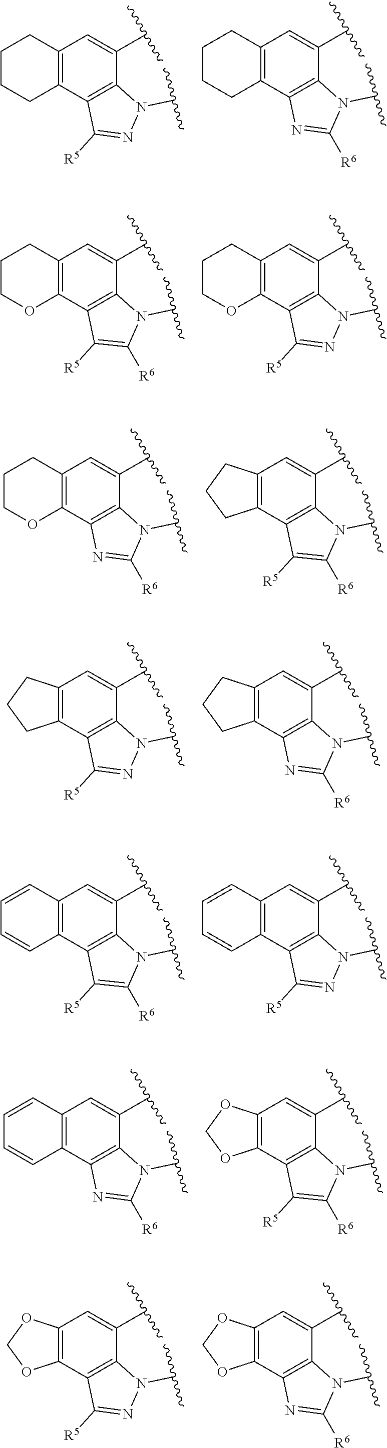 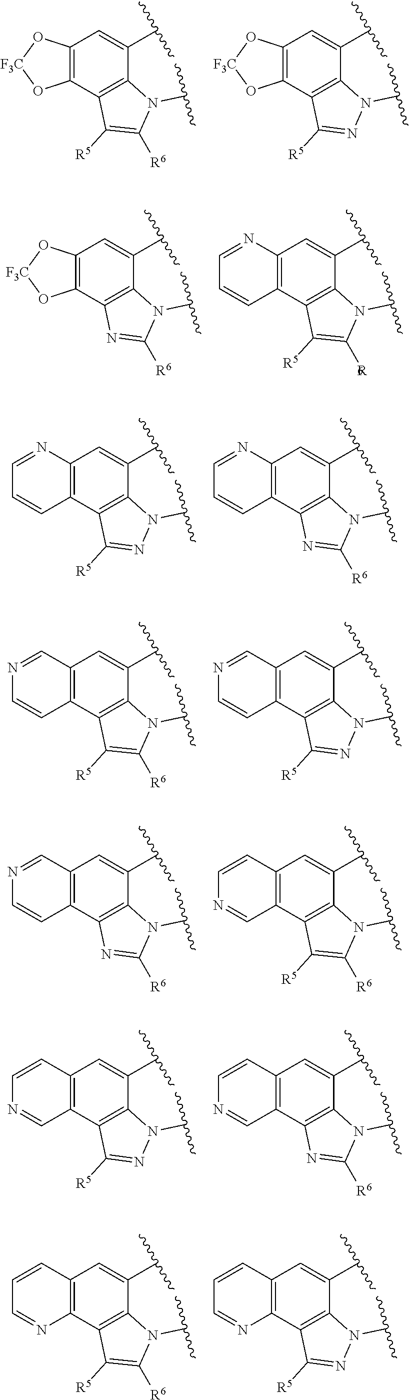 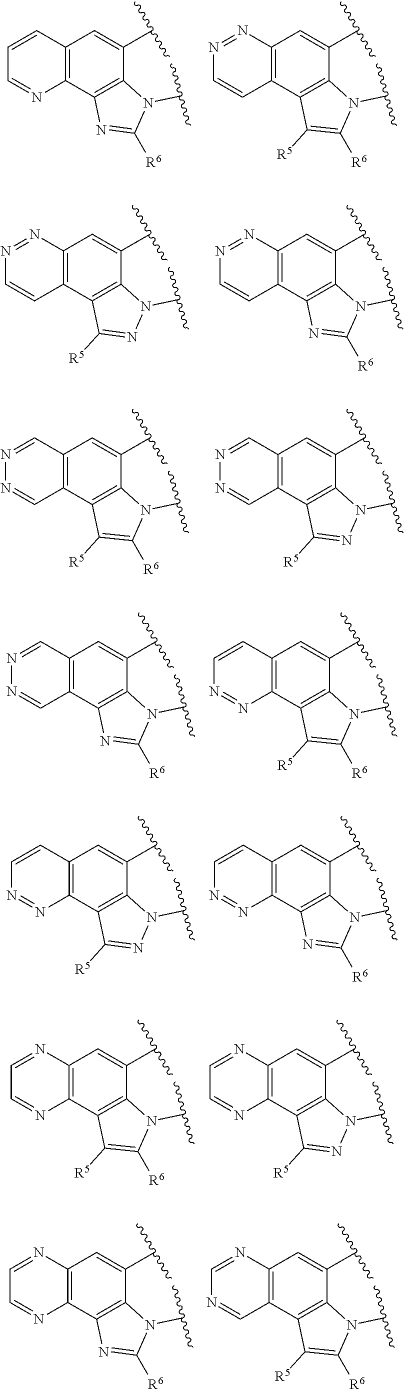 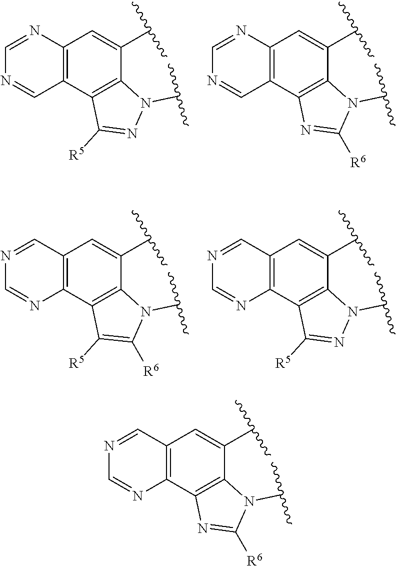
Z is O, S or NR⁸;
X¹ is N or CR⁴;
X², X³ are independently N, CR⁴ or a carbon atom which is connected with —Y²—Ar² with a proviso that at least one of X² or X³ is a carbon atom which is connected with —Y²—Ar²;
X⁴ is O, S, N, NR⁵ or CR⁵;
X⁵ is O, S, N, CR⁶ or —C(O)—;
X⁶, X⁷ and X⁸ are independently N or C;
Y² is —C(O)—, —(CR^{a}R^{1a})ₙ—, —CH═CH, —C≡C—, —O—, —S—, S(O)₂—, —S(O)₂NR^{b}—, —NR^{b}S(O)₂—, —C(O)NR^{b}—, —NR^{b}C(O)—, NR^{b}C(O)NR^{b}—, NR^{b}S(O)₂NR^{b}—, —NR^{b}— or Y² is absent and Ar² is directly connected X² or X³;
Y¹ is —O—, —S—, —NR^{b}—, —C(O)—, or —CR^{a}R^{1a}—;
Y³ is —(CR^{a}₂)ₘ—;
n and m are independently 0, 1, 2 or 3; r is 1 or 2;
each R^{a} and R^{1a} are independently hydrogen, halogen, C₁₋₆alkyl or C₁₋₆haloalkyl, or two R^{a} and R^{1a}, together with the carbon atom to which they are attached to form a 3- to 6-menbered cycloalkyl or heterocyclyl;
R⁴, R⁵, and R⁶ are each independently hydrogen, alkyl, halogen, —OR⁸, —NR⁸R⁹, —SR⁹, —S(O)₂NR⁸R⁹, —S(O)₂R¹¹, —CN, C₃₋₆cyclolkyl, —C₀₋₆alkylene-aryl, —C₀₋₆alkylene-heteroaryl or haloalkyl;
Ar¹ and Ar² are independently selected from the group consisting of: —OR′, —SR′, NR′R⁹, C₁₋₁₀alkyl, C₁₋₁₀haloalkyl, —C₀₋₆alkylene-C₃₋₆cycloalkyl, —C₀₋₆alkylene-aryl, —C₀₋₆alkylene-heteroaryl and —C₀₋₆alkylene-heterocyclyl, or a fused analog of C₃₋₆cycloalkyl; wherein Ar¹ and Ar² are independently substituted with one to three R^{c} groups;
two Ar² on the adjacent carbon atoms together with carbon atoms can form a three- to six-membered carbocyclic ring, four- to eight-membered bicyclic ring, C₅ to C₁₀ aromatic ring which is optionally substituted with one or more R^{c}, C₂ to C₁₀ heteroaromatic ring which is unsubstituted or substituted with one or more R^{c} or a three- to six-membered carbocyclic ring, four- to eight-membered bicyclic ring or five- to eight-membered spirocyclic ring which contains one, two or three heteroatom(s) such as S, O or NR^{b};
each R^{e} is independently halogen, hydrogen, C₁₋₆alkyl, C₃₋₆cycloalkyl, C₁₋₆fluorocycloalkyl, C₁₋₆fluoroalkyl, —C₂-C₉alkynyl, —C₀₋₆alkylene-heterocyclyl, —C₀₋₆alkylene-aryl, —C₀₋₆alkylene-heteroaryl, SF₅, —C₀₋₆alkylene-OR⁸, —C₀₋₆alkylene-P(O)Me2, —C₀₋₆alkylene-NR′R⁹, —C₀₋₆alkylene-C(O)NR⁸R⁹, —C₀₋₆alkylene-NR⁸(O)NR⁸R⁹, —C₀₋₆alkylene-SR′, —C₀₋₆alkylene-S(O)₂NR⁸R⁹, —C₀₋₆alkylene-S(O)₂R¹⁰ or —C₀₋₆alkylene-CN, wherein C₁₋₆ alkyl, C₂₋₉ alkenyl, C₂₋₉ alkynyl, —C₀₋₆alkylene-cycloalkyl, —C₀₋₆alkylene-heterocyclyl, —C₀₋₆alkylene-aryl, and —C₀₋₆alkylene-heteroaryl; R^{c}unsubstituted or substituted with one, two, or three R^{V};
R^{v} is independently alkyl, haloalkyl, cycloalkyl, cycloalkylalkyl, heterocyclyl, heterocyclylalkyl, halogen, —OR⁸, —NR′R⁹, —CN, —C(O)R¹⁰, —C(O)NR′R⁹, —NR′C(O)R¹⁰, —NR′C(O)OR⁹, —NR¹⁰ C(O)NR⁸R⁹, —OC(O)NR′R⁹, —S(O)₂R¹⁰, —S(O)R¹⁰, —SR′, —S(O)₂NR⁸R⁹, —S(O)NR⁸R⁹, —NR⁸S(O)R¹⁰, —NR⁸S(O)₂R¹⁰, or —NR¹⁰ S(O)₂NR⁸R⁹; wherein alkyl is unsubstituted or substituted with —OR⁸ or —NR⁸R⁹ and wherein cycloalkyl and heterocyclyl are optionally substituted with one, two, or three groups selected from halogen, alkyl, and haloalkyl;
R⁷ is selected from C₁₋₆alkyl, C₁₋₆haloalkyl, C₃₋₆cycloalkyl, C₃₋₆cyclohaloalkyl, aryl and heteroaryl;
each R′ and each R⁹ are independently hydrogen, alkyl, haloalkyl, cycloalkyl, heterocyclyl, aryl, or heteroaryl, wherein cycloalkyl, heterocyclyl, aryl, and heteroaryl are optionally substituted with one, two, or three groups selected from halogen, alkyl, and haloalkyl; or R¹ and R⁹, together with the atom or atoms to which they are attached, form a heterocyclyl unsubstituted or substituted with one, two, or three groups selected from halogen, alkyl, and haloalkyl;
each R¹⁰ is independently alkyl, haloalkyl, cycloalkyl, heterocyclyl, aryl, or heteroaryl, wherein cycloalkyl, heterocyclyl, aryl, and heteroaryl are unsubstituted or substituted with one, two, or three groups selected from halogen, alkyl, and haloalkyl; 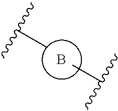
is selected from the following structures: 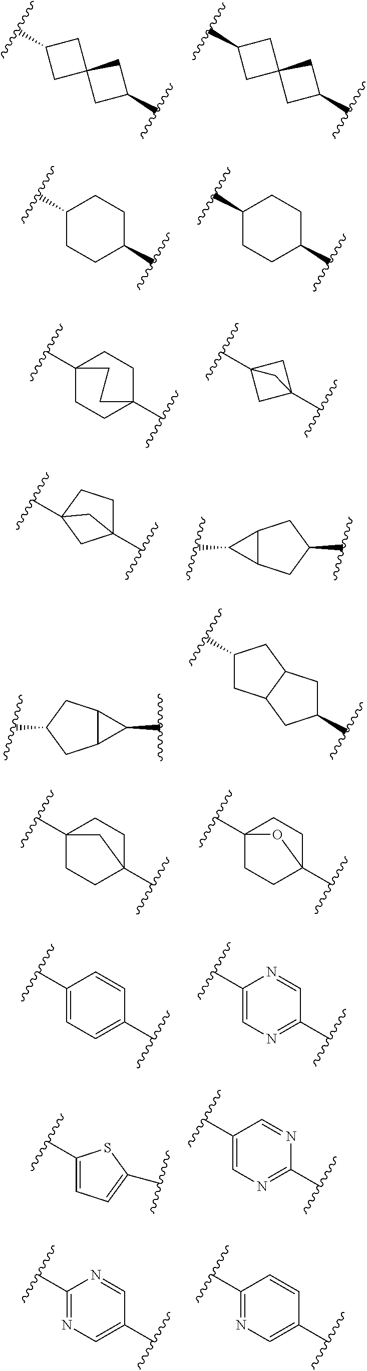 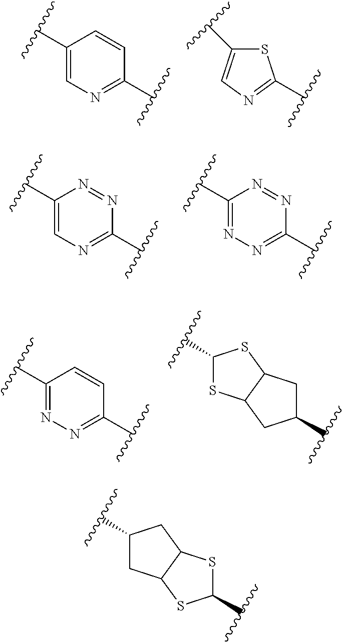 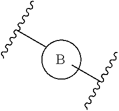
is unsubstituted or substituted with one or more R^{e}.

17. The compound according to claim 16, wherein said compound of Formula I is 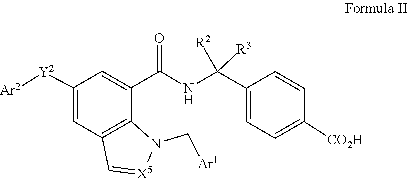
or a pharmaceutically acceptable salt thereof, wherein X⁵ is N or CR⁶; R², R³, R⁶, Y², Ar¹ and Ar² are defined as in Formula I, when R² and R³ are different groups, Formula II includes both R and S.

18. The compound according to claim 16, wherein said compound of Formula I is 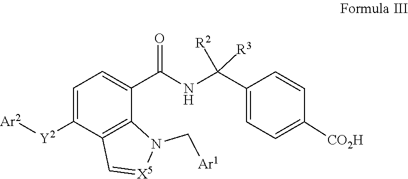
wherein X⁵ is N or CR⁶; R², R³, R⁶, Y², Ar¹ and Ar² are defined as in Formula I, when R² and R³ are different groups, Formula III includes both R and S enantiomers.

19. The compound according to claim 16, wherein said compound of Formula I is 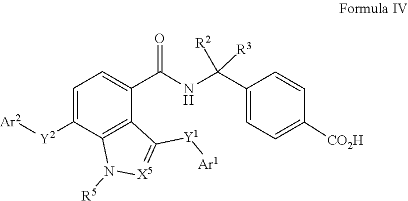
wherein: X⁵ is N or CR⁶; R², R³, R⁵, R⁶, Yi, Y², Ar¹ and Ar² are defined as in Formula I, when R² and R³ are different groups, Formula IV includes both R and S enantiomers.

20. The compound according to claim 16, wherein said compound of Formula I is 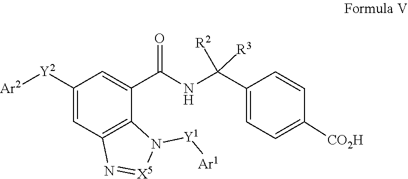
wherein X⁵ is N or CR⁶; R², R³, R⁶, Yi, Y², Ar¹ and Ar² are defined as in Formula I, when R² and R³ are different groups, Formula V includes both R and S enantiomers.

21. The compound according to claim 16, wherein said compound of Formula I is 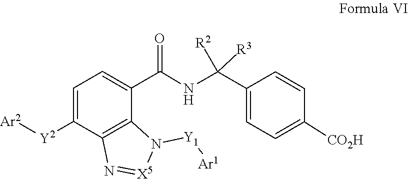
wherein X⁵ is N or CR⁶; R², R³, R⁶, Y¹, Y², Ar¹ and Ar² are defined as in Formula I, when R² and R³ are different groups, Formula VI includes both R and S enantiomers.

22. The compound according to claim 16, wherein said compound of Formula I is 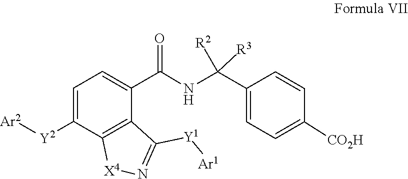
wherein: X⁴ is NR⁵, O or S; R², R³, R⁵, Y¹, Y², Ar¹ and Ar² are defined as in Formula I, when R² and R³ are different groups, Formula VII includes both R and S enantiomers.

23. The compound according to claim 16, wherein said compound of Formula I is 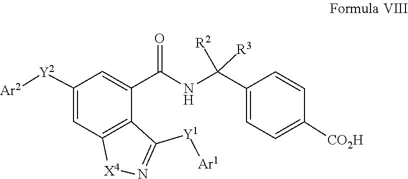
wherein X⁴ is NR⁵, O or S; R², R³, R⁵, Y¹, Y², Ar¹ and Ar² are defined as in Formula I, when R² and R³ are different groups, Formula VIII includes both R and S enantiomers.

24. The compound according to claim 16, wherein said compound of Formula I is 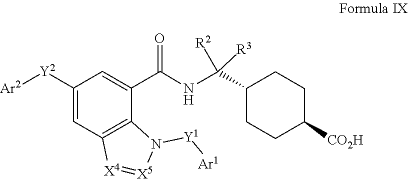
wherein X⁴ and X⁵ are independently N or CR⁶; R², R³, R⁶, Y¹, Y², Ar¹ and Ar² are defined as in Formula I, when R² and R³ are different groups, Formula IX includes both R and S enantiomers.

25. The compound according to claim 16, wherein said compound of Formula I is 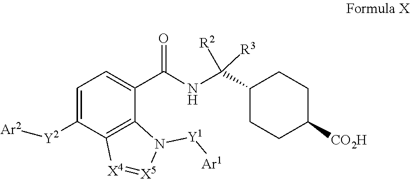
wherein X⁴ and X⁵ are independently N or CR⁶; R², R³, R⁶, Yi, Y², Ar¹ and Ar² are defined as in Formula I, when R² and R³ are different groups, Formula X includes both R and S enantiomers.

26. The compound according to claim 16, wherein said compound of Formula I is 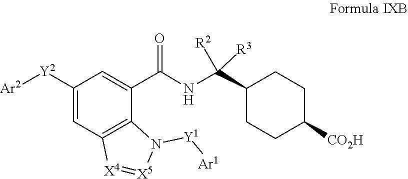
wherein X⁴ and, X⁵ is are independently N or CR⁶; R², R³, R⁶, Yi, Y², Ar¹ and Ar² are defined as in Formula I, when R² and R³ are different groups, Formula IXB includes both R and S enantiomers.

27. The compound according to claim 16, wherein said compound of Formula I is 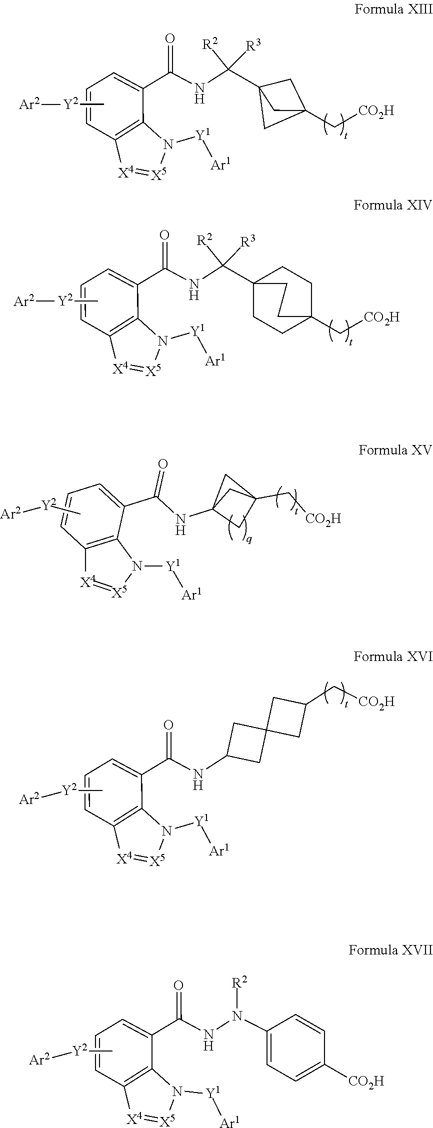
wherein X⁴ and X⁵ are independently N or CR⁶; R², R³, R⁶, Yi, Y², Ar¹ and Ar² are defined as in Formula I; t is 0, 1 or 2; q is 1, 2 or 3, when R² and R³ are different groups, Formula XIII and XIV include both R and S enantiomers.

28. The compound of claim 16, or a pharmaceutically acceptable salt thereof, wherein the compound is selected from the group consisting of

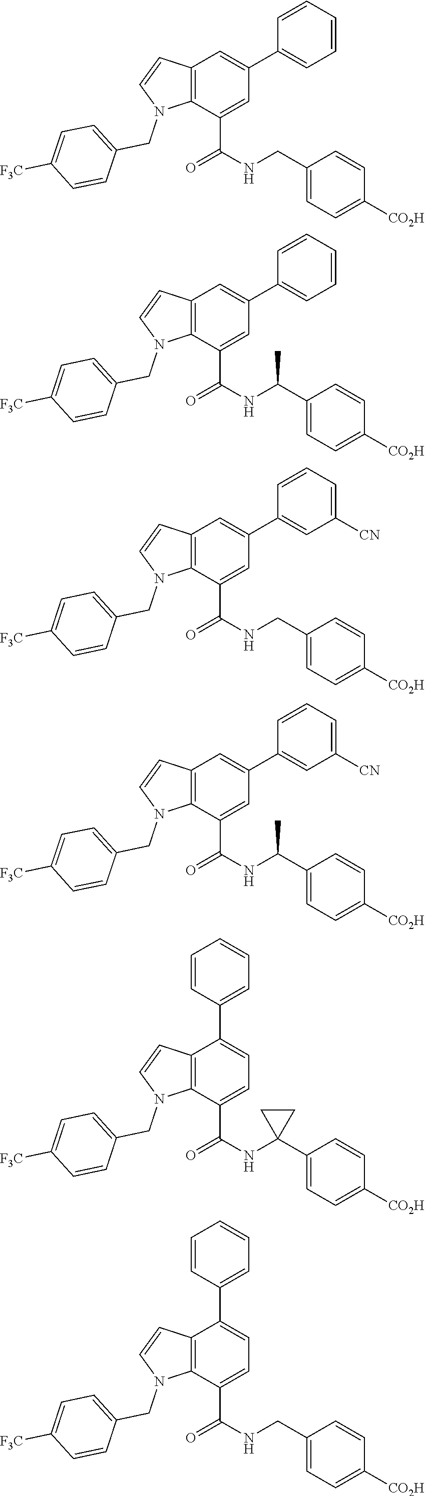

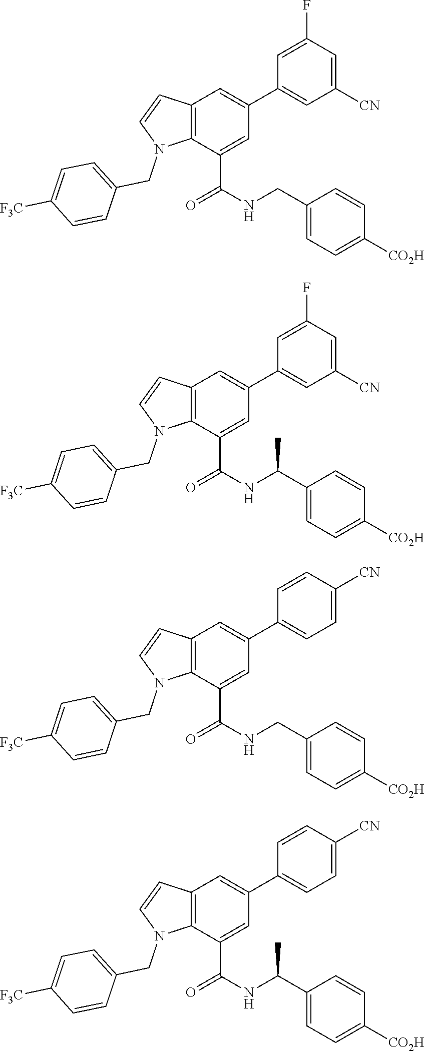

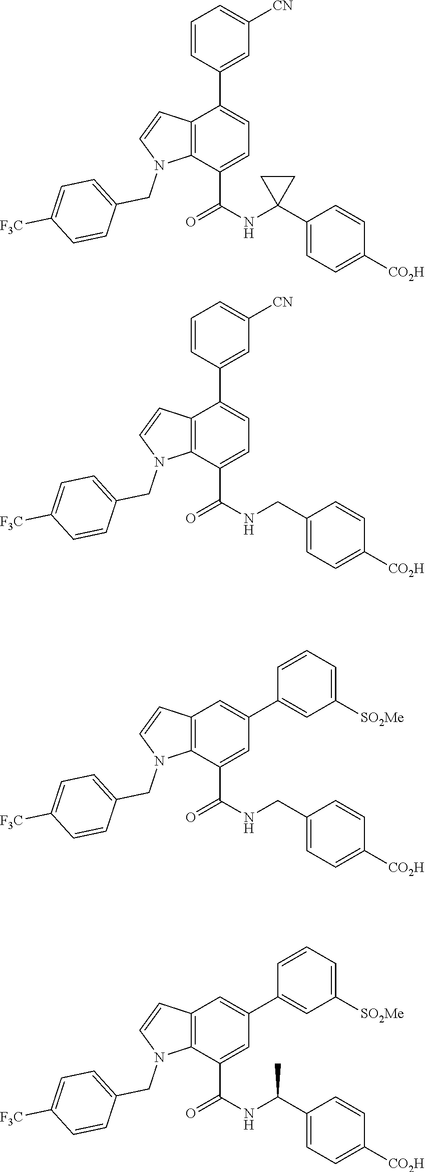 [SMILES: N#Cc1cccc(-c2ccc(C(=O)NC3(c4ccc(C(=O)O)cc4)CC3)c3c2ccn3Cc2ccc(C(F)(F)F)cc2)c1 ; N#Cc1cccc(-c2ccc(C(=O)NCc3ccc(C(=O)O)cc3)c3c2ccn3Cc2ccc(C(F)(F)F)cc2)c1 ; CS(=O)(=O)c1cccc(-c2cc(C(=O)NCc3ccc(C(=O)O)cc3)c3c(ccn3Cc3ccc(C(F)(F)F)cc3)c2)c1 ; C[C@H](NC(=O)c1cc(-c2cccc(S(C)(=O)=O)c2)cc2ccn(Cc3ccc(C(F)(F)F)cc3)c12)c1ccc(C(=O)O)cc1]

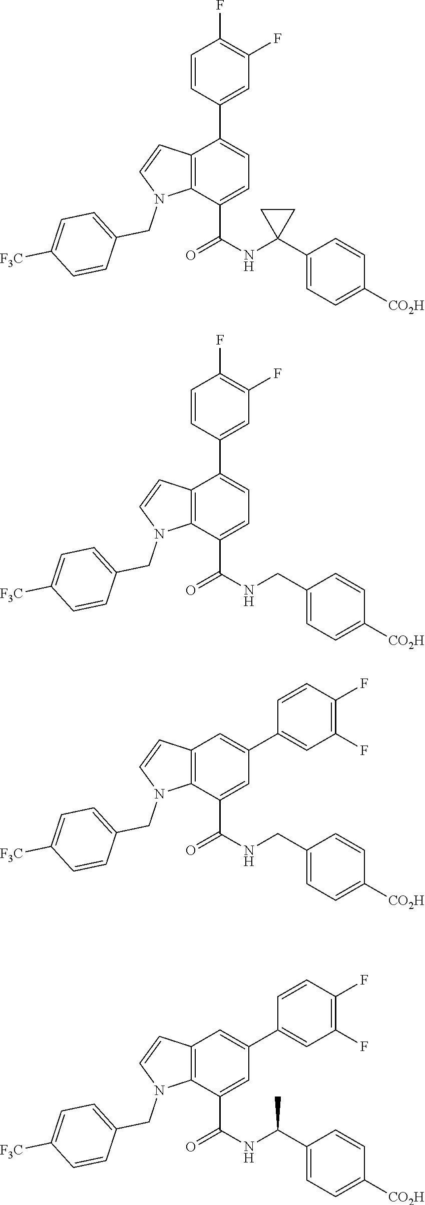 [SMILES: O=C(O)c1ccc(C2(NC(=O)c3ccc(-c4ccc(F)c(F)c4)c4ccn(Cc5ccc(C(F)(F)F)cc5)c34)CC2)cc1 ; O=C(O)c1ccc(CNC(=O)c2ccc(-c3ccc(F)c(F)c3)c3ccn(Cc4ccc(C(F)(F)F)cc4)c23)cc1 ; O=C(O)c1ccc(CNC(=O)c2cc(-c3ccc(F)c(F)c3)cc3ccn(Cc4ccc(C(F)(F)F)cc4)c23)cc1 ; C[C@H](NC(=O)c1cc(-c2ccc(F)c(F)c2)cc2ccn(Cc3ccc(C(F)(F)F)cc3)c12)c1ccc(C(=O)O)cc1]

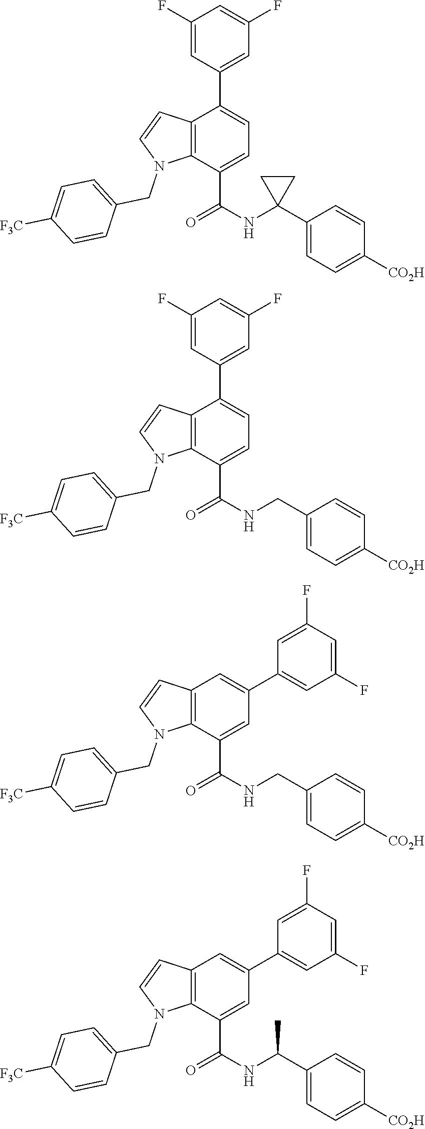

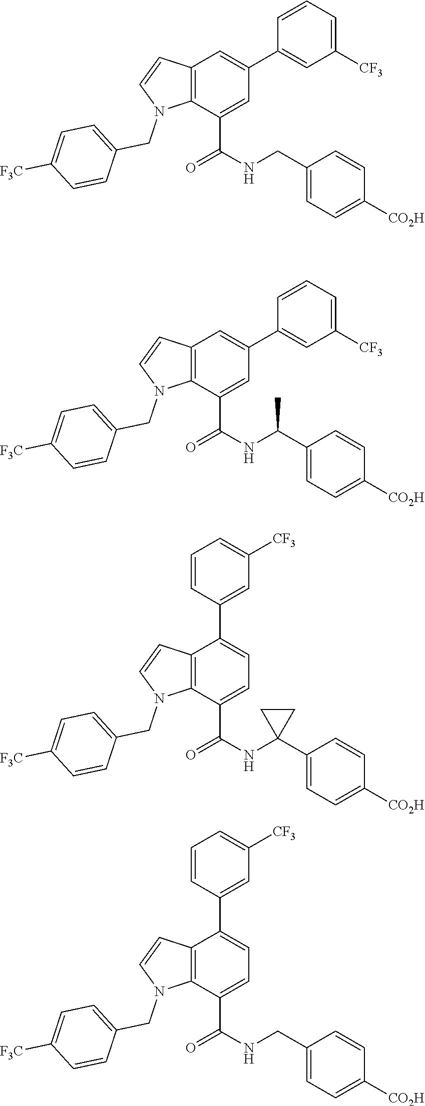 [SMILES: O=C(O)c1ccc(CNC(=O)c2cc(-c3cccc(C(F)(F)F)c3)cc3ccn(Cc4ccc(C(F)(F)F)cc4)c23)cc1 ; C[C@H](NC(=O)c1cc(-c2cccc(C(F)(F)F)c2)cc2ccn(Cc3ccc(C(F)(F)F)cc3)c12)c1ccc(C(=O)O)cc1 ; O=C(O)c1ccc(C2(NC(=O)c3ccc(-c4cccc(C(F)(F)F)c4)c4ccn(Cc5ccc(C(F)(F)F)cc5)c34)CC2)cc1 ; O=C(O)c1ccc(CNC(=O)c2ccc(-c3cccc(C(F)(F)F)c3)c3ccn(Cc4ccc(C(F)(F)F)cc4)c23)cc1]

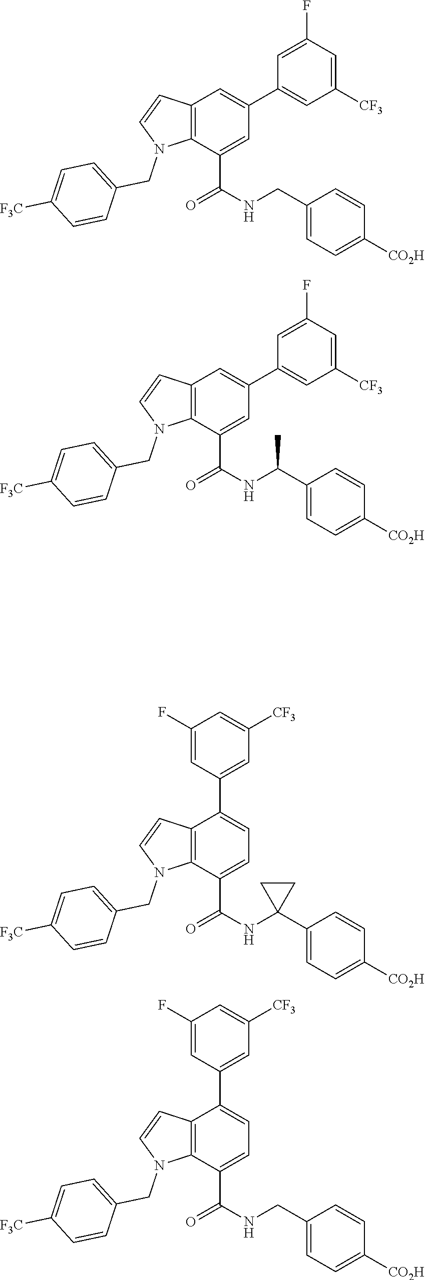

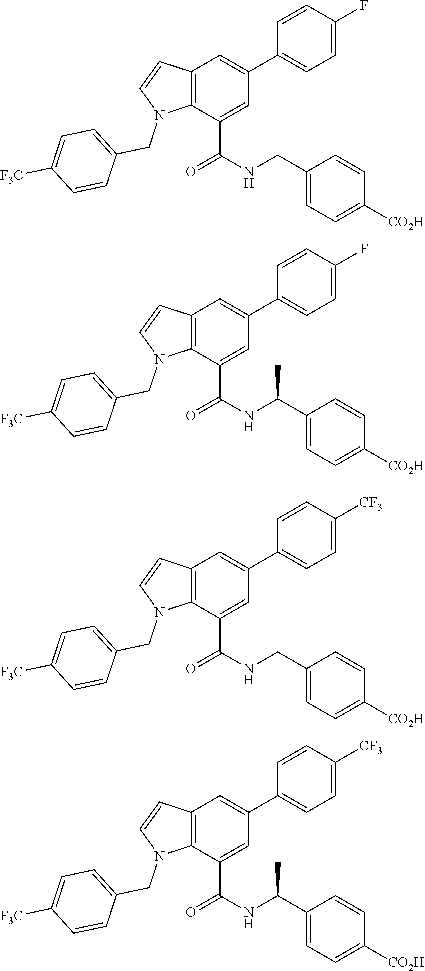

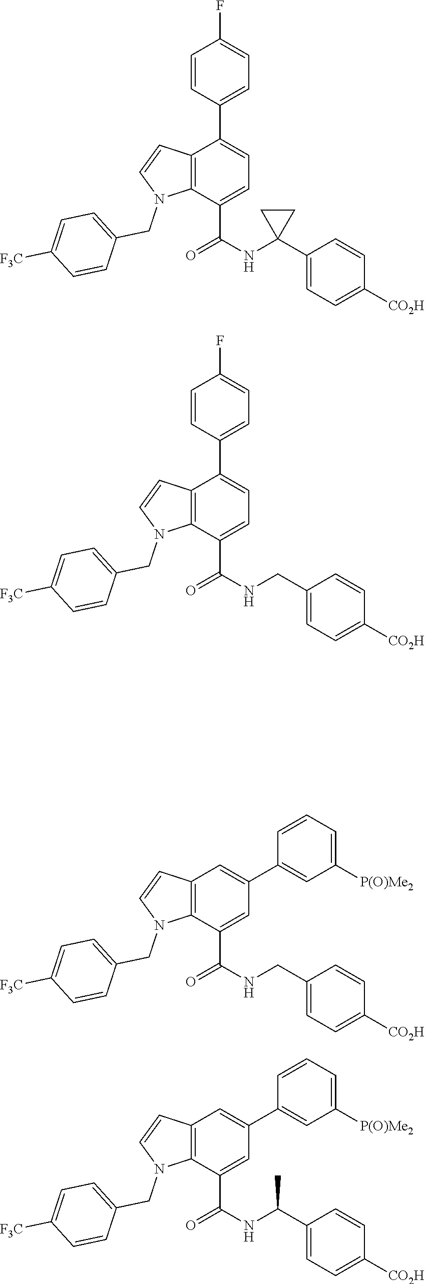

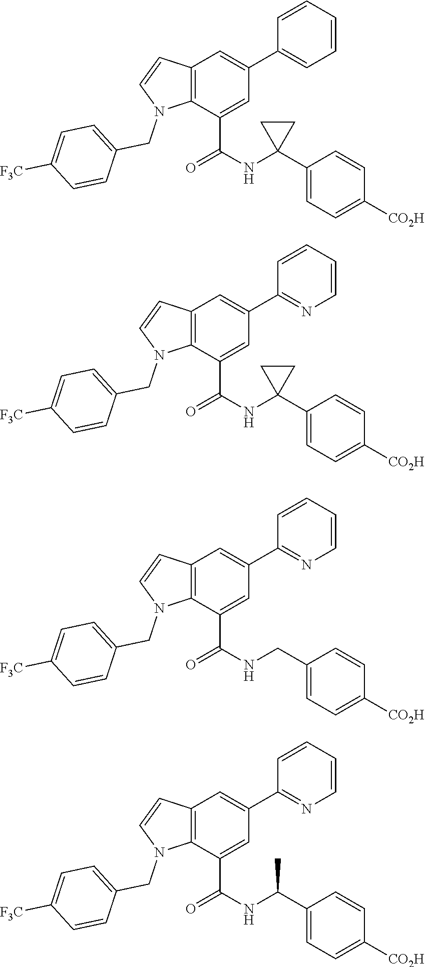

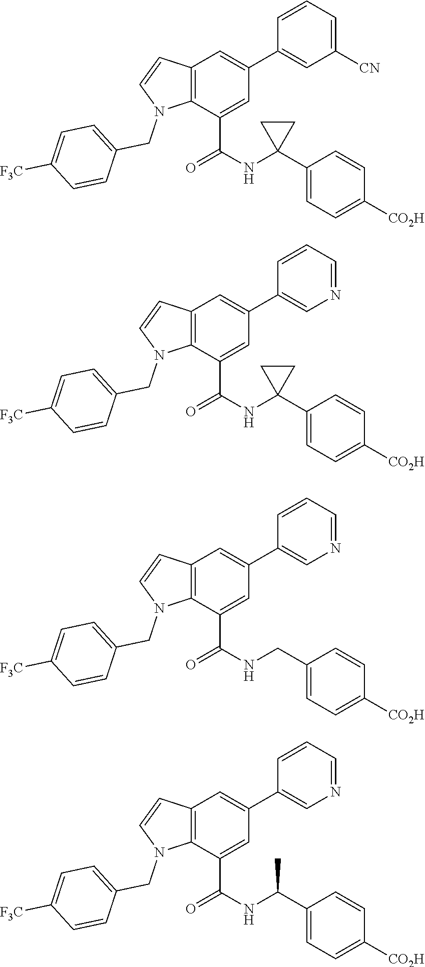

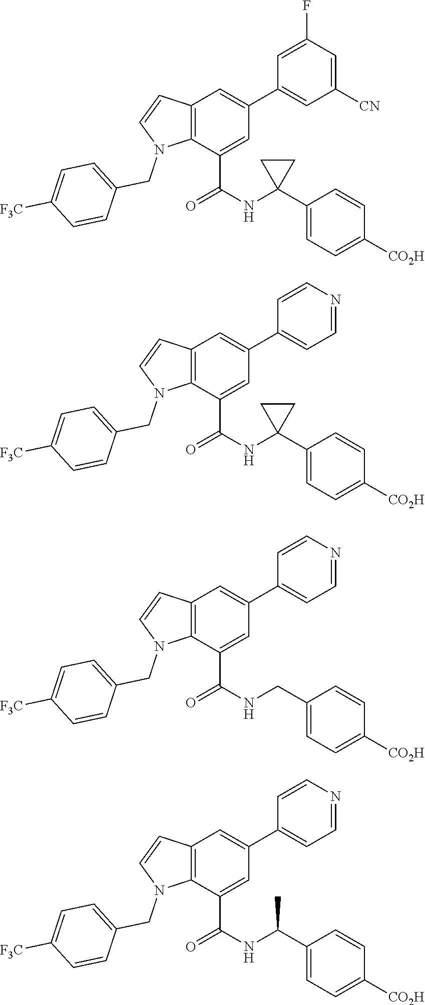

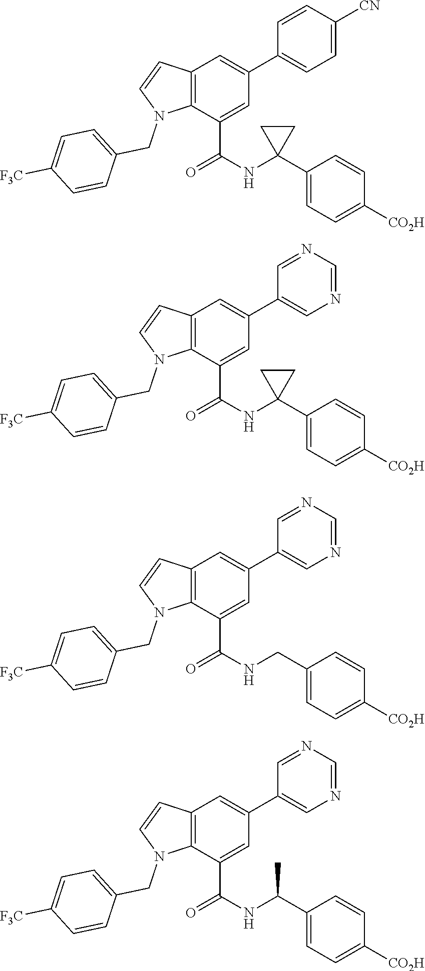

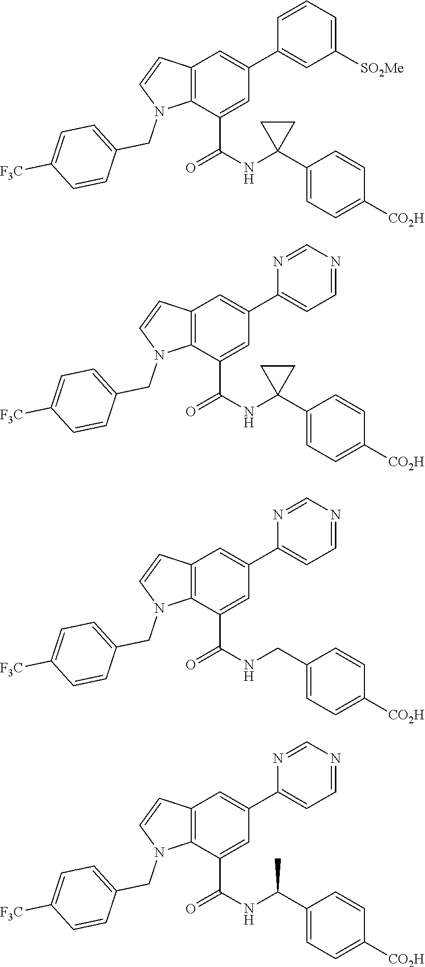

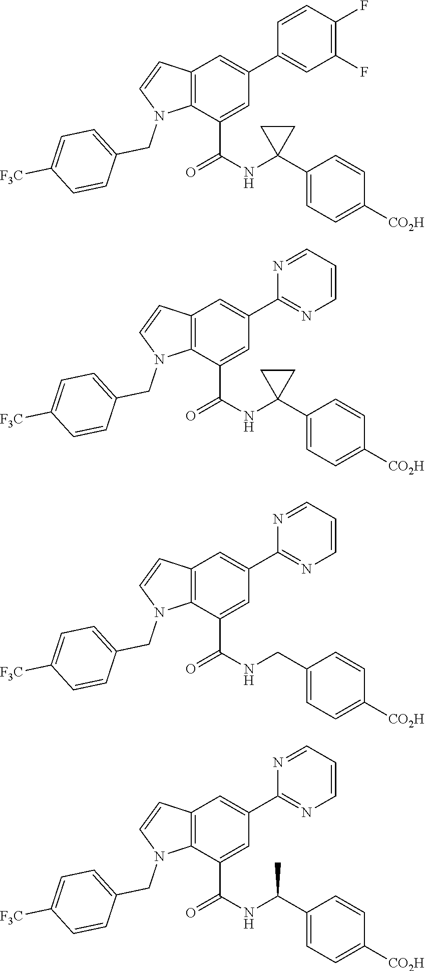

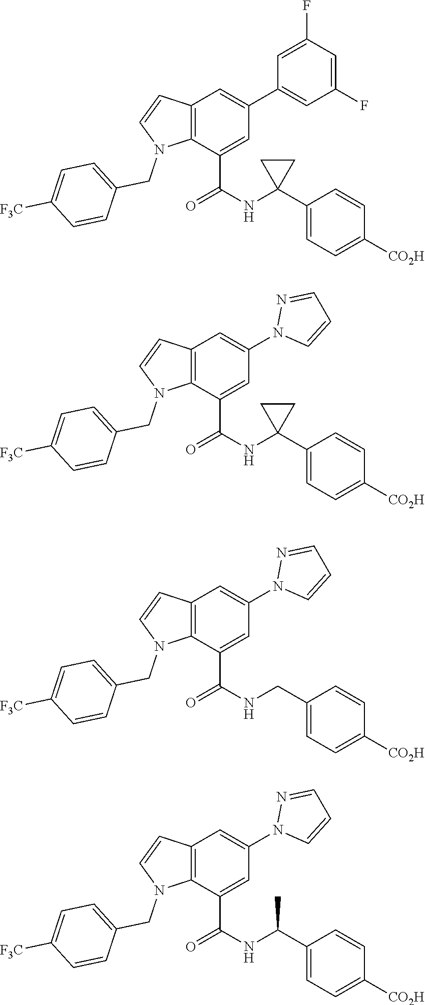

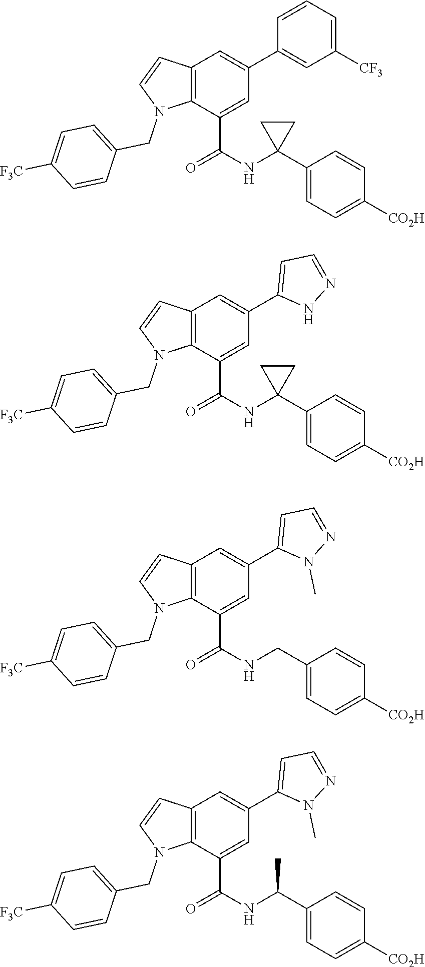

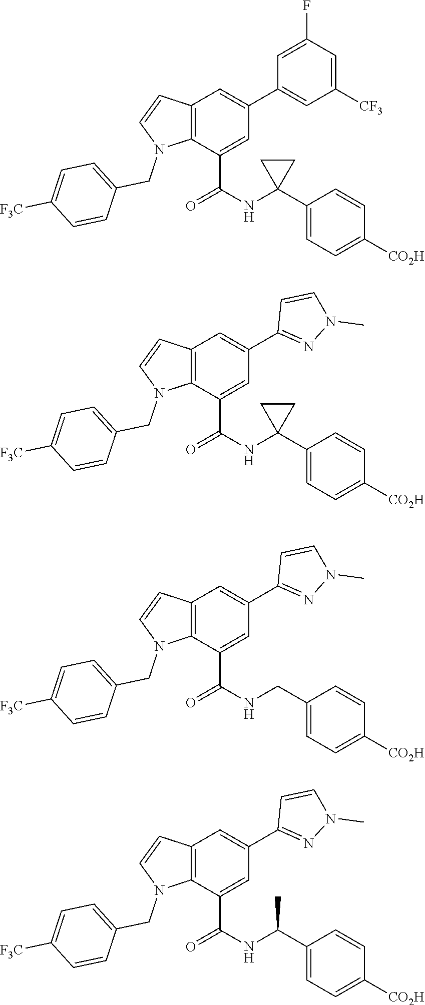

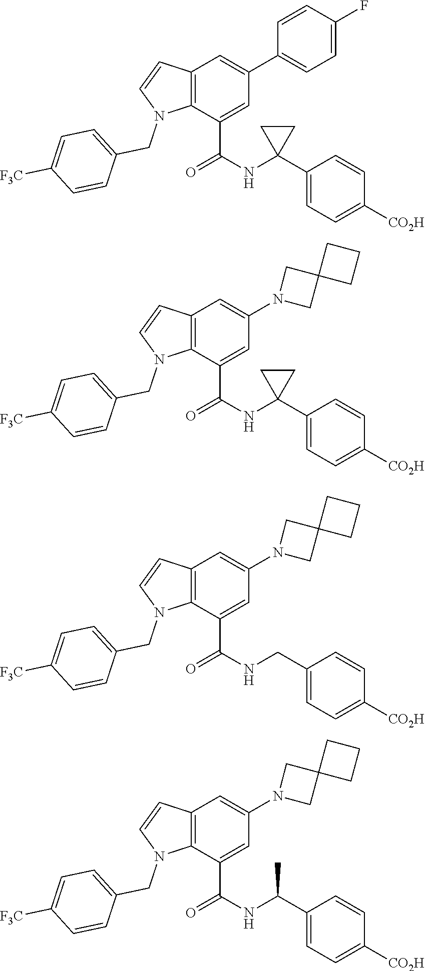

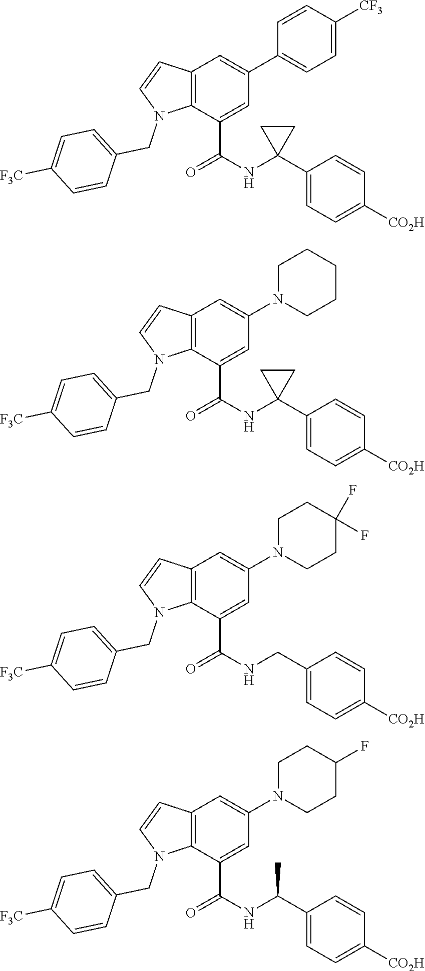

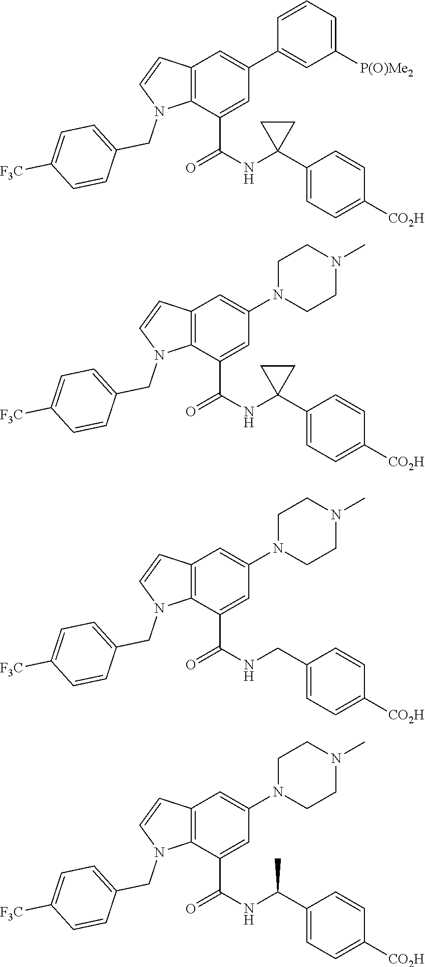

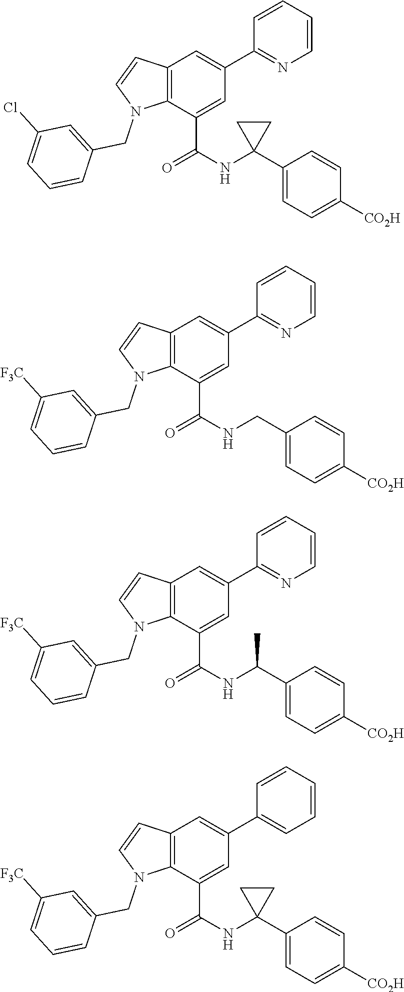

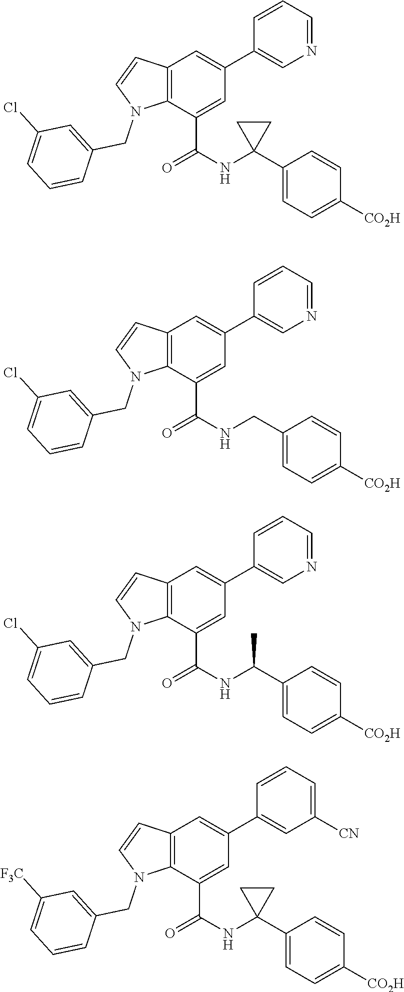

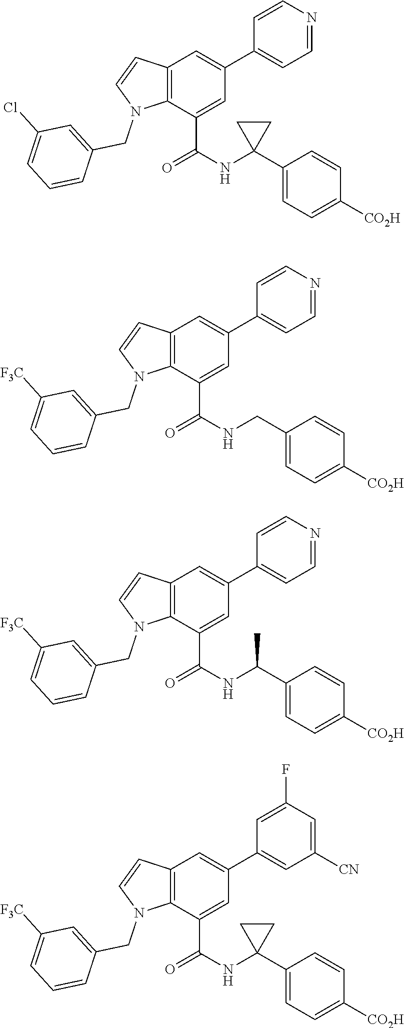

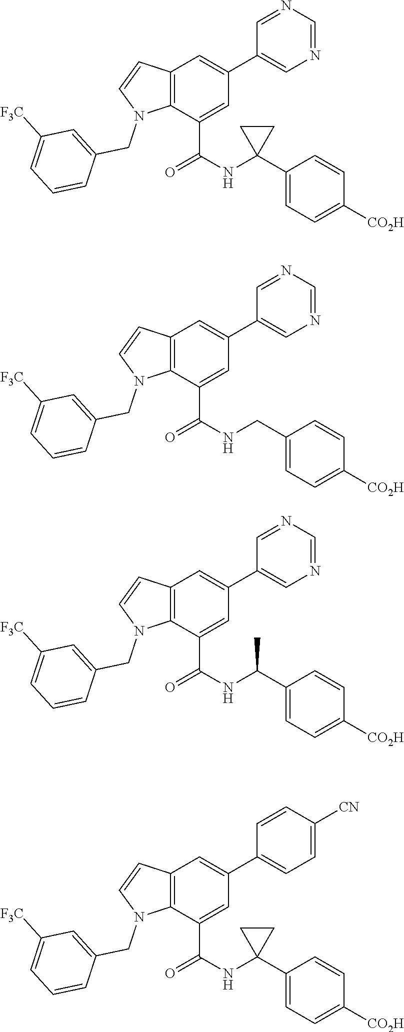

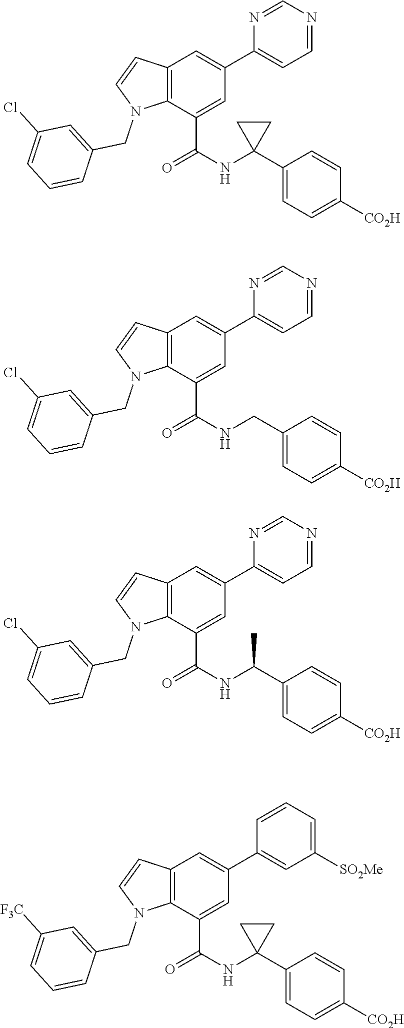

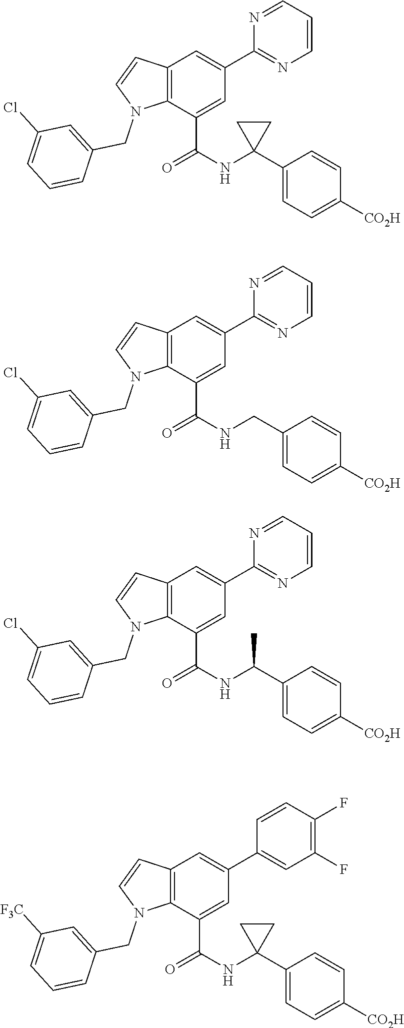

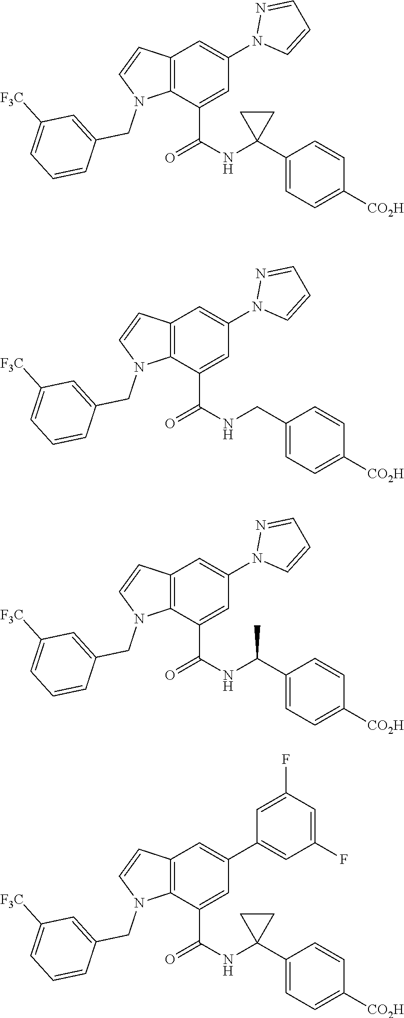

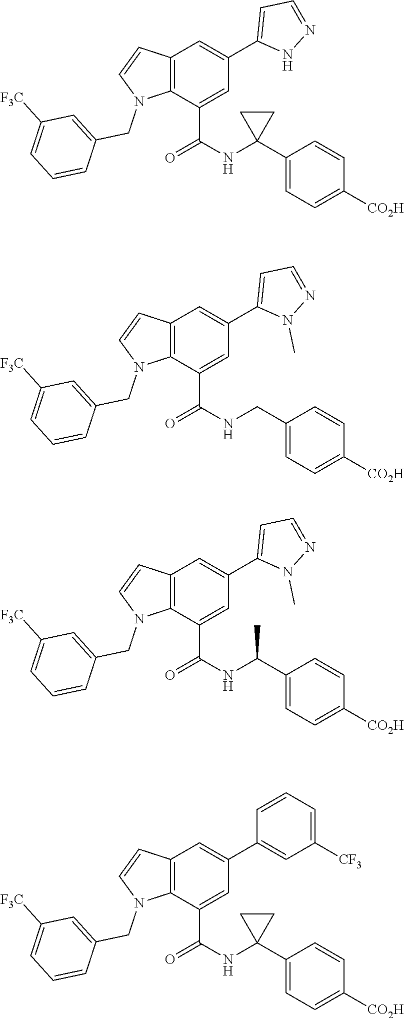 [SMILES: O=C(O)c1ccc(C2(NC(=O)c3cc(-c4ccn[nH]4)cc4ccn(Cc5cccc(C(F)(F)F)c5)c34)CC2)cc1 ; Cn1nccc1-c1cc(C(=O)NCc2ccc(C(=O)O)cc2)c2c(ccn2Cc2cccc(C(F)(F)F)c2)c1 ; C[C@H](NC(=O)c1cc(-c2ccnn2C)cc2ccn(Cc3cccc(C(F)(F)F)c3)c12)c1ccc(C(=O)O)cc1 ; O=C(O)c1ccc(C2(NC(=O)c3cc(-c4cccc(C(F)(F)F)c4)cc4ccn(Cc5cccc(C(F)(F)F)c5)c34)CC2)cc1]

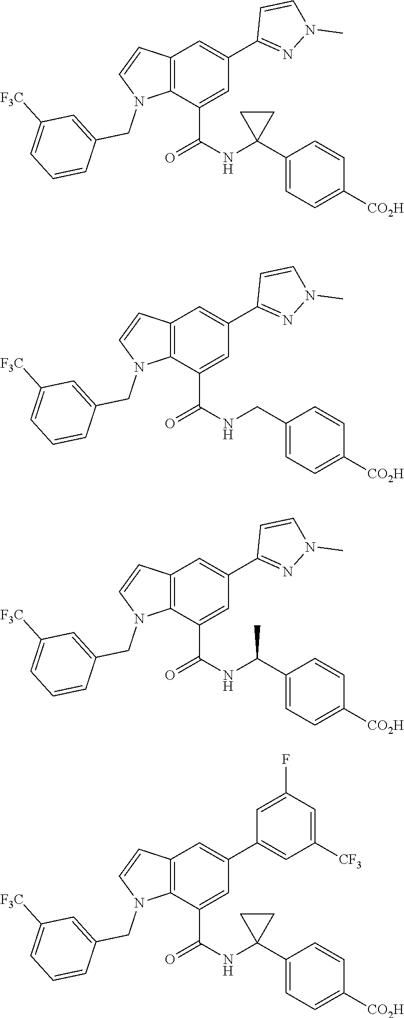

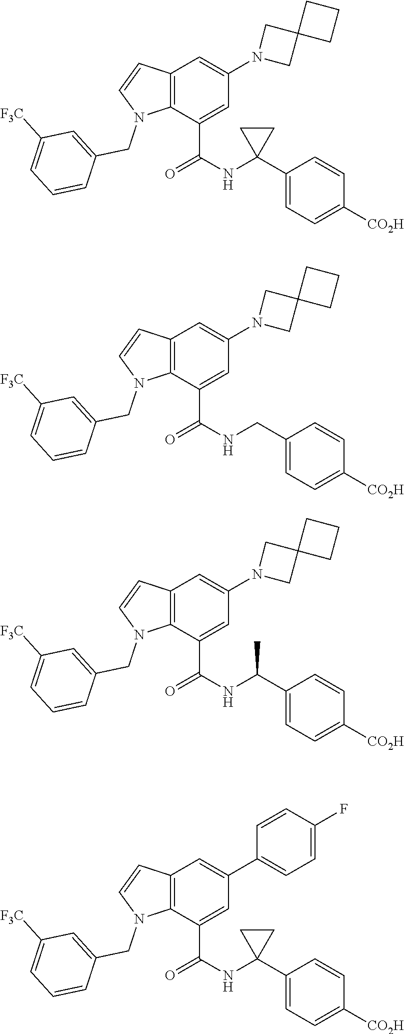

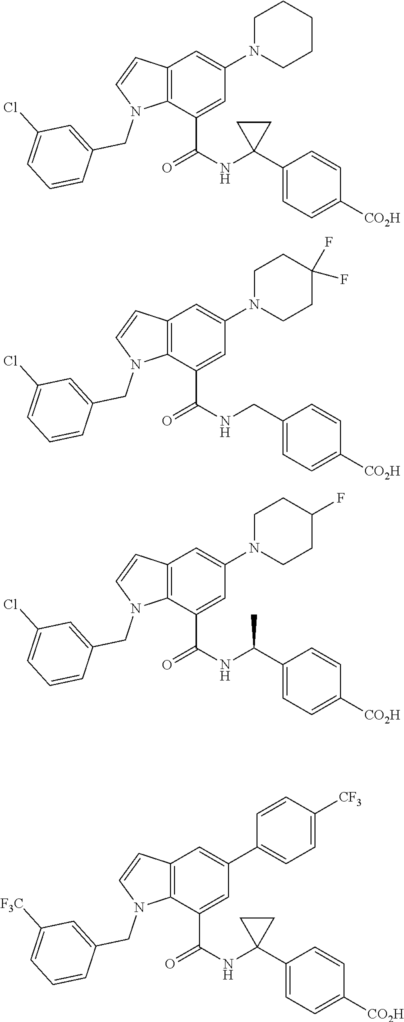

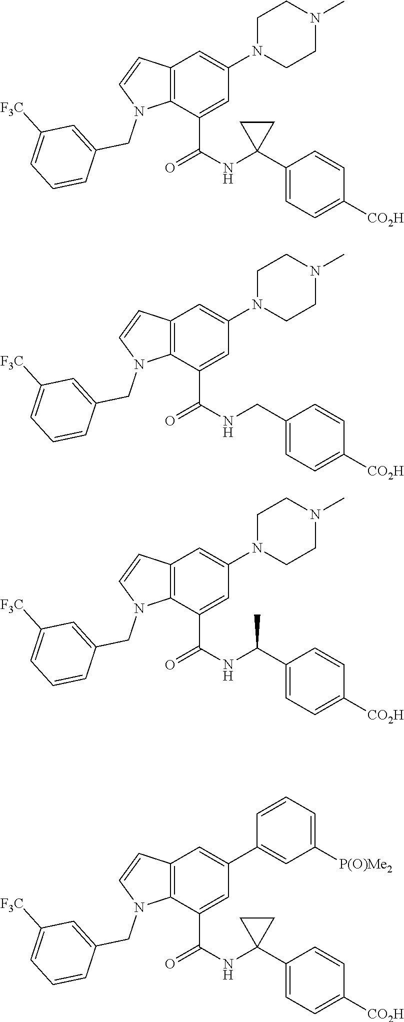

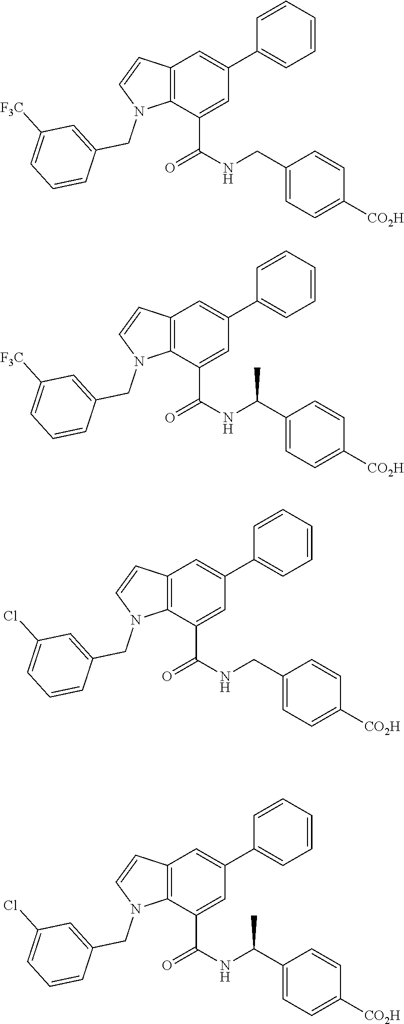

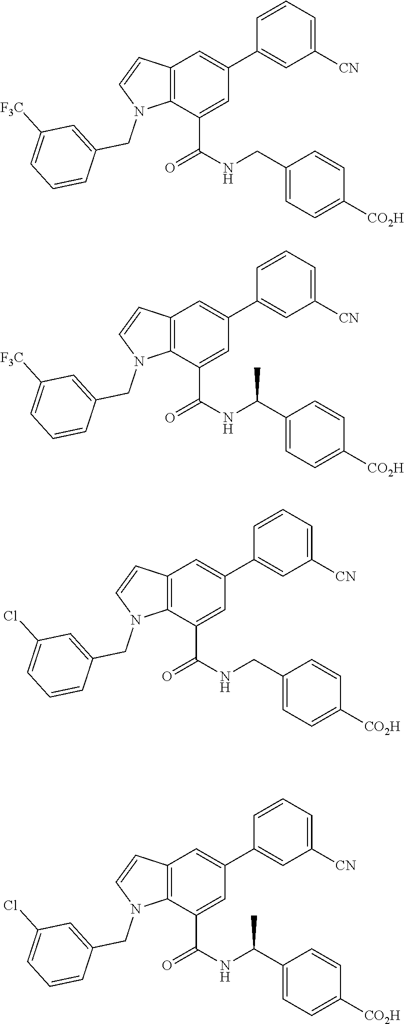

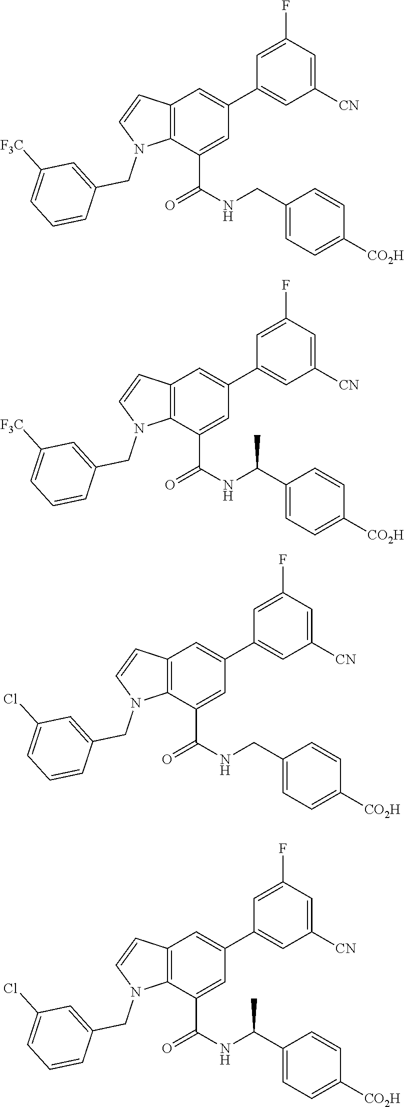

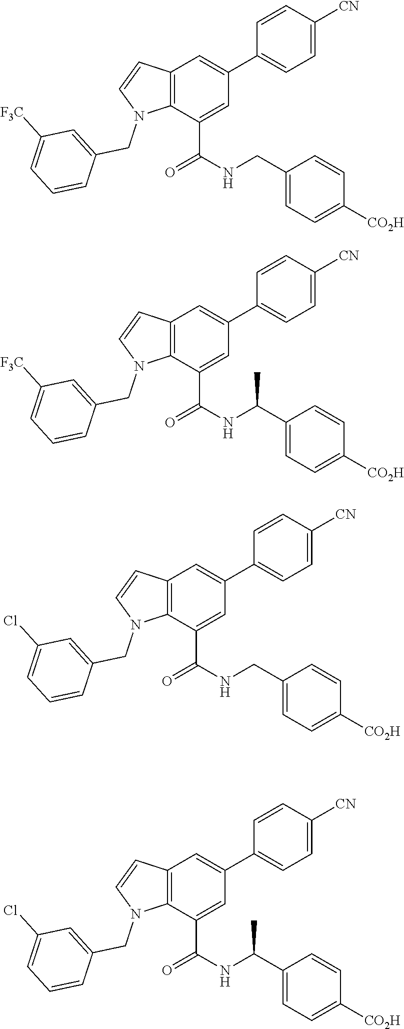

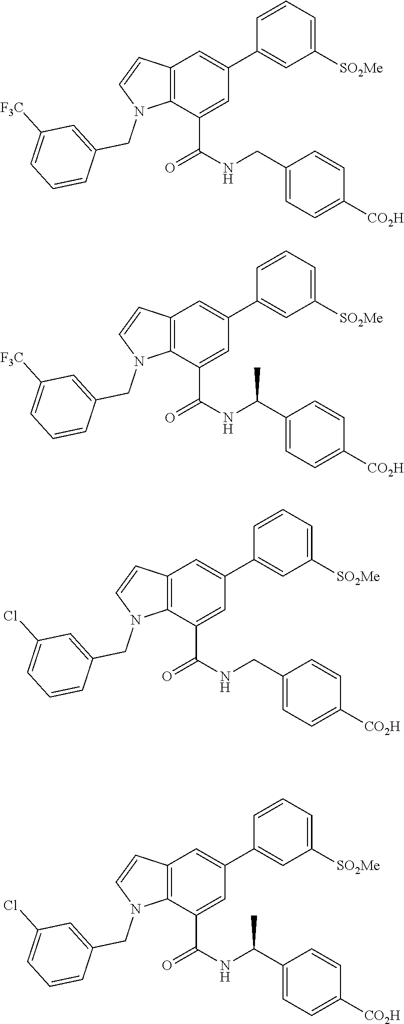

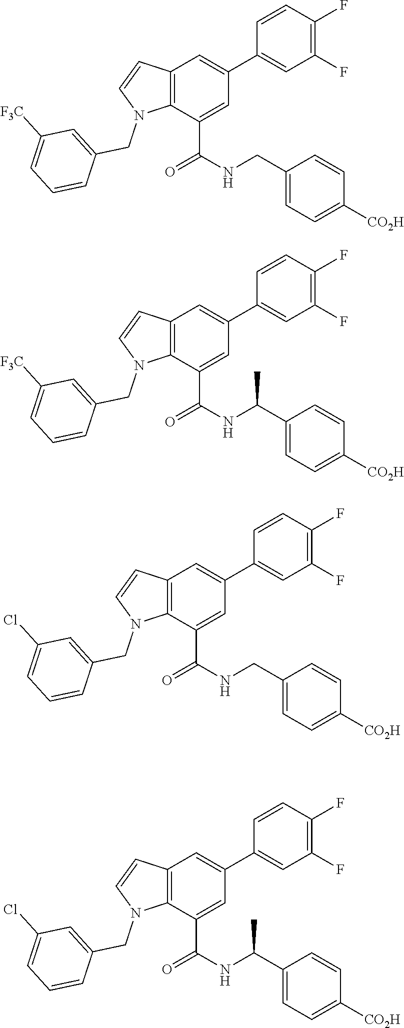

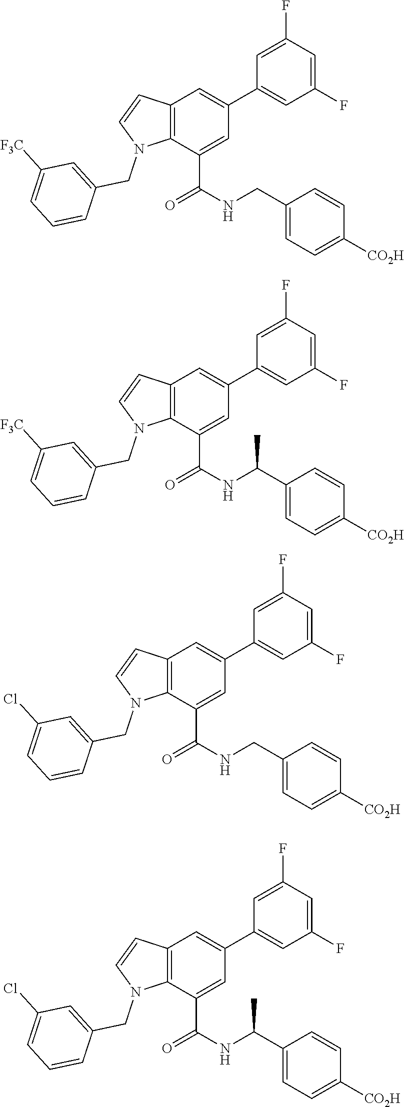

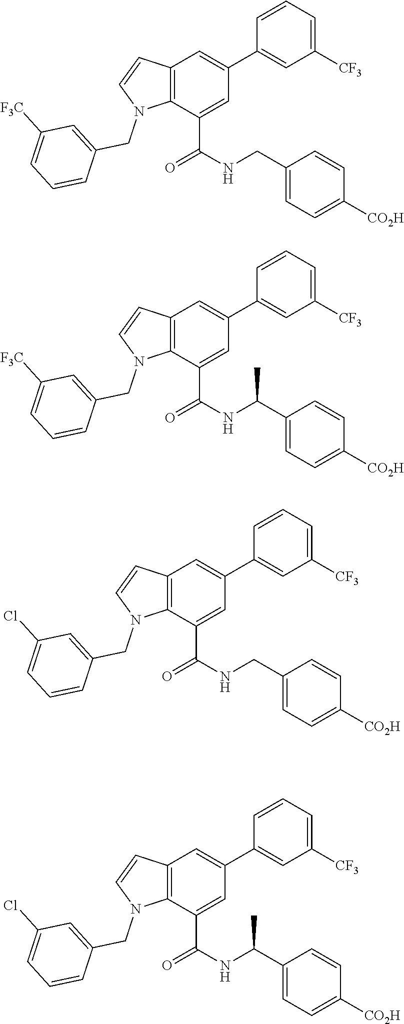

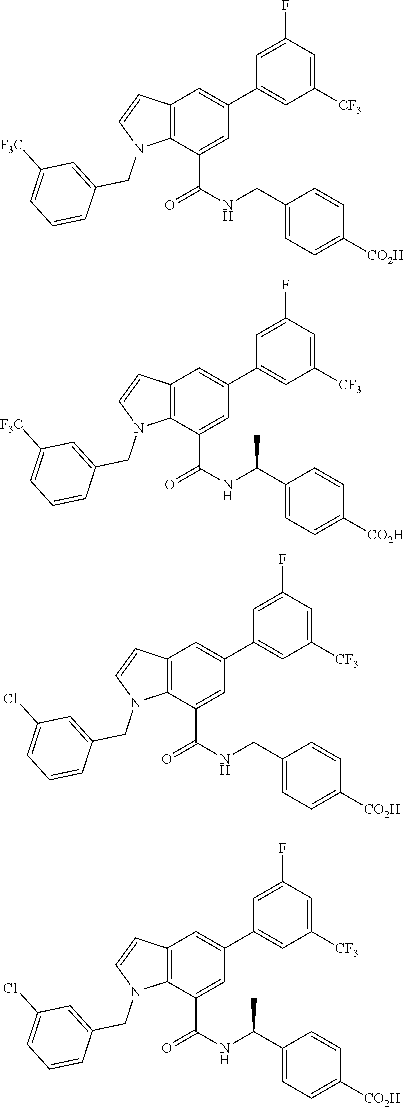

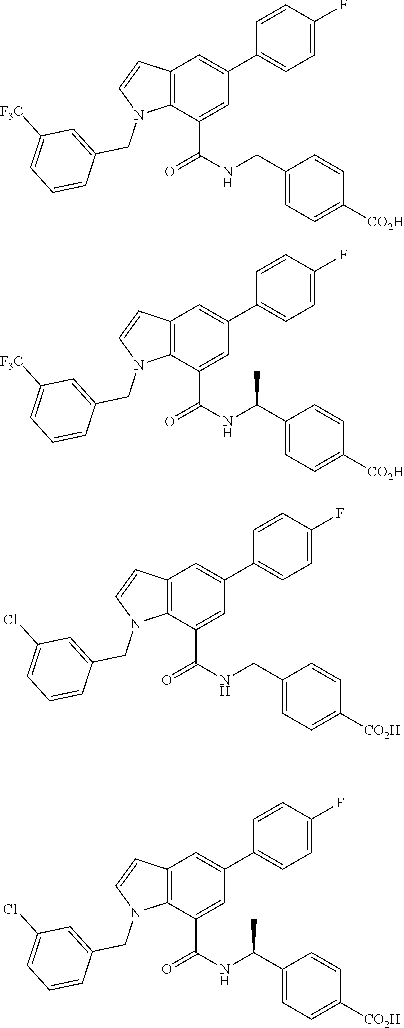

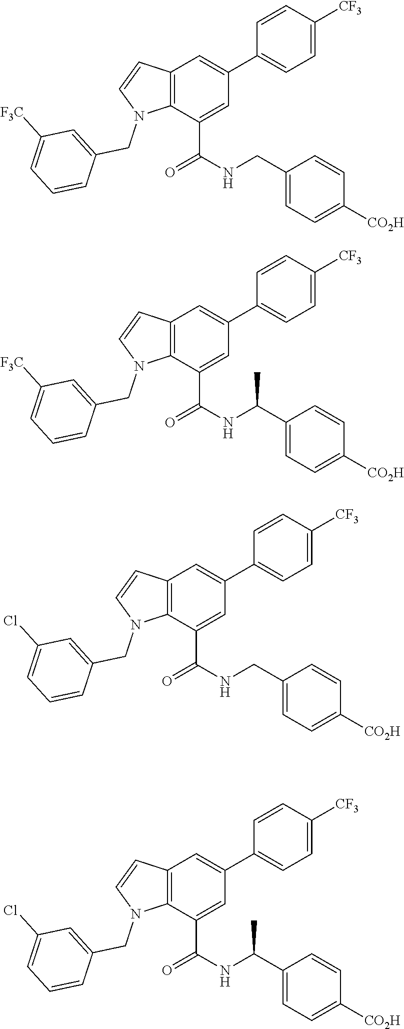

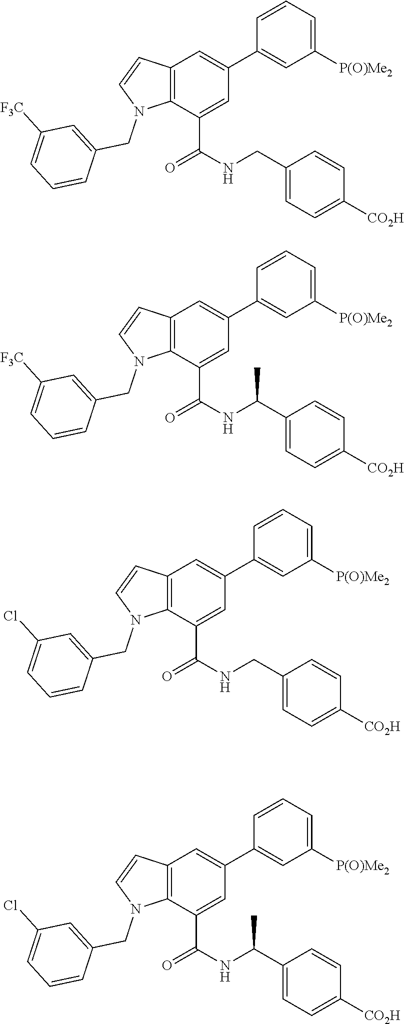

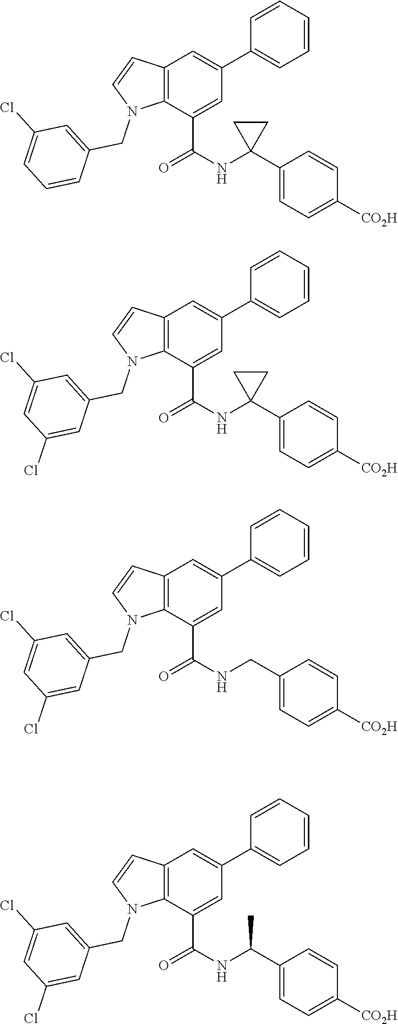

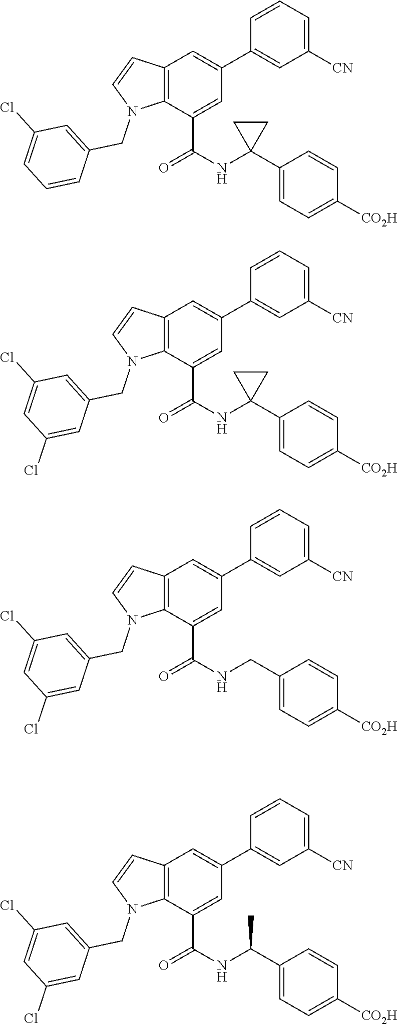

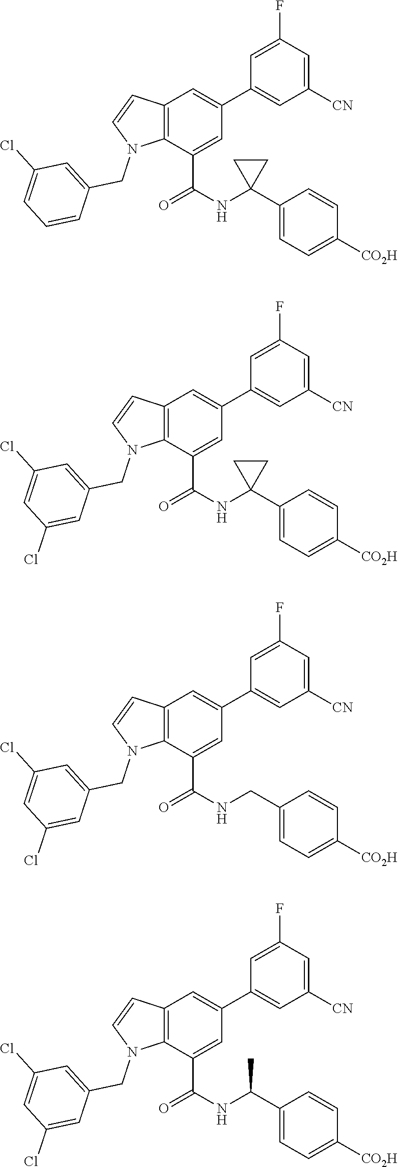 [SMILES: N#Cc1cc(F)cc(-c2cc(C(=O)NC3(c4ccc(C(=O)O)cc4)CC3)c3c(ccn3Cc3cccc(Cl)c3)c2)c1 ; N#Cc1cc(F)cc(-c2cc(C(=O)NC3(c4ccc(C(=O)O)cc4)CC3)c3c(ccn3Cc3cc(Cl)cc(Cl)c3)c2)c1 ; N#Cc1cc(F)cc(-c2cc(C(=O)NCc3ccc(C(=O)O)cc3)c3c(ccn3Cc3cc(Cl)cc(Cl)c3)c2)c1 ; C[C@H](NC(=O)c1cc(-c2cc(F)cc(C#N)c2)cc2ccn(Cc3cc(Cl)cc(Cl)c3)c12)c1ccc(C(=O)O)cc1]

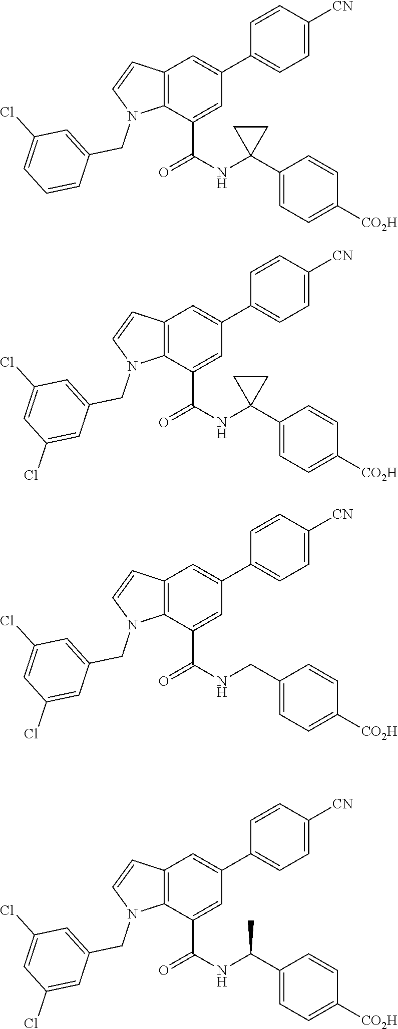

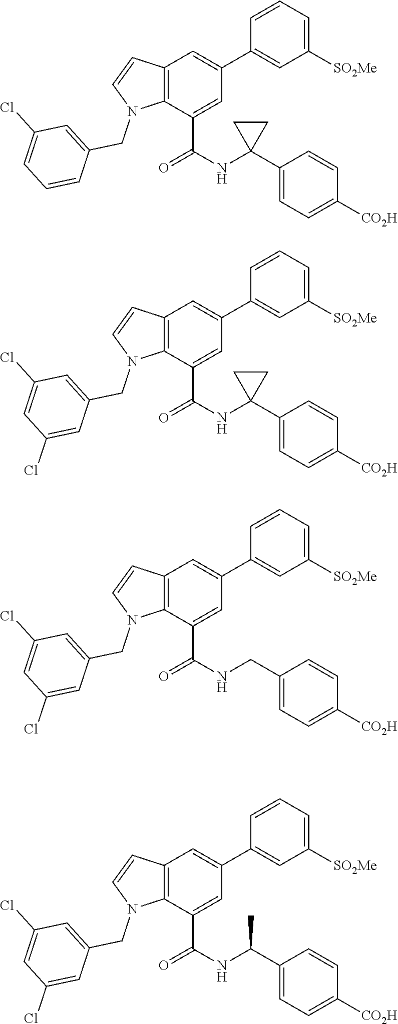

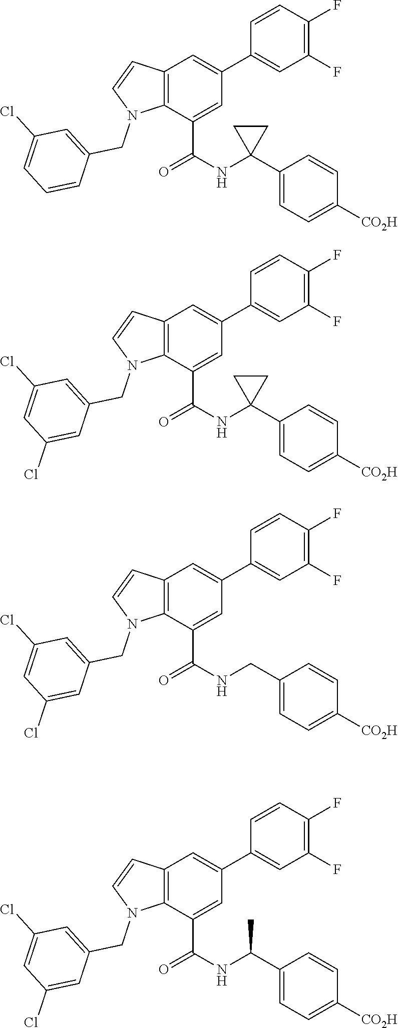

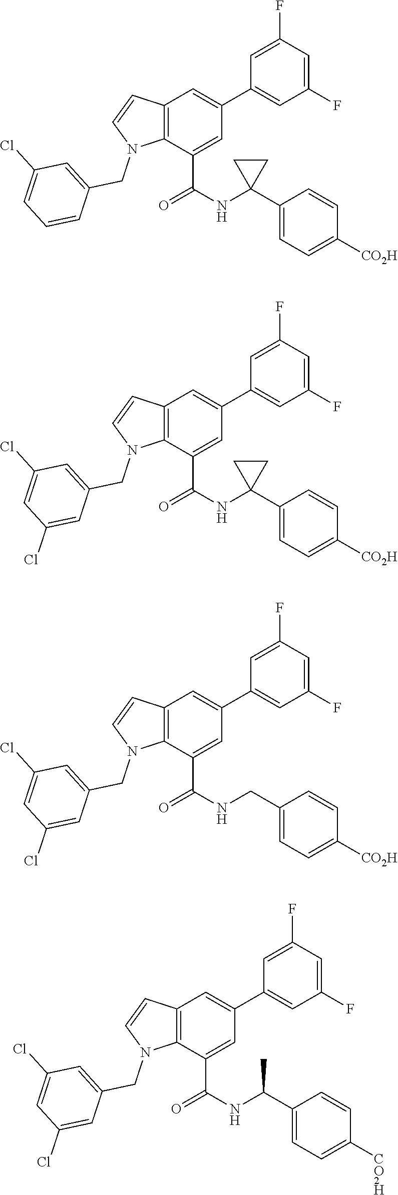 [SMILES: O=C(O)c1ccc(C2(NC(=O)c3cc(-c4cc(F)cc(F)c4)cc4ccn(Cc5cccc(Cl)c5)c34)CC2)cc1 ; O=C(O)c1ccc(C2(NC(=O)c3cc(-c4cc(F)cc(F)c4)cc4ccn(Cc5cc(Cl)cc(Cl)c5)c34)CC2)cc1 ; O=C(O)c1ccc(CNC(=O)c2cc(-c3cc(F)cc(F)c3)cc3ccn(Cc4cc(Cl)cc(Cl)c4)c23)cc1 ; C[C@H](NC(=O)c1cc(-c2cc(F)cc(F)c2)cc2ccn(Cc3cc(Cl)cc(Cl)c3)c12)c1ccc(C(=O)O)cc1]

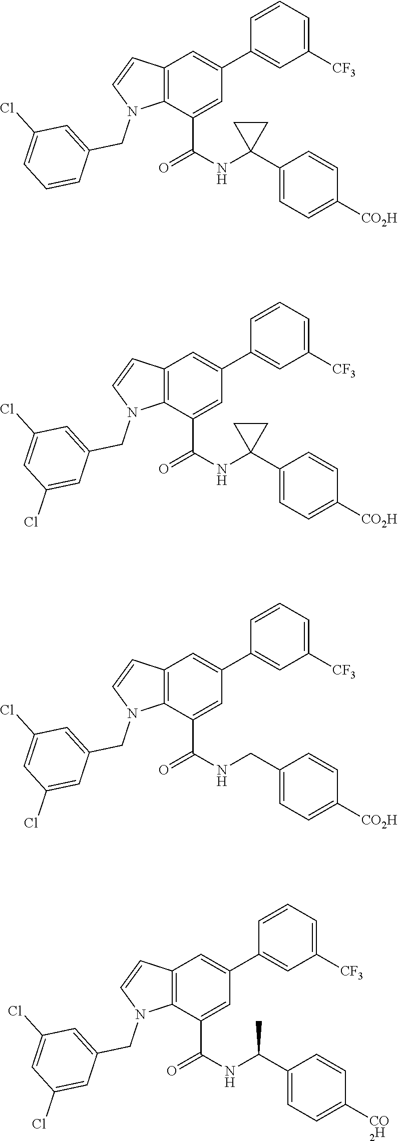 [SMILES: O=C(O)c1ccc(C2(NC(=O)c3cc(-c4cccc(C(F)(F)F)c4)cc4ccn(Cc5cccc(Cl)c5)c34)CC2)cc1 ; O=C(O)c1ccc(C2(NC(=O)c3cc(-c4cccc(C(F)(F)F)c4)cc4ccn(Cc5cc(Cl)cc(Cl)c5)c34)CC2)cc1 ; O=C(O)c1ccc(CNC(=O)c2cc(-c3cccc(C(F)(F)F)c3)cc3ccn(Cc4cc(Cl)cc(Cl)c4)c23)cc1 ; C[C@H](NC(=O)c1cc(-c2cccc(C(F)(F)F)c2)cc2ccn(Cc3cc(Cl)cc(Cl)c3)c12)c1ccc(C(=O)O)cc1]

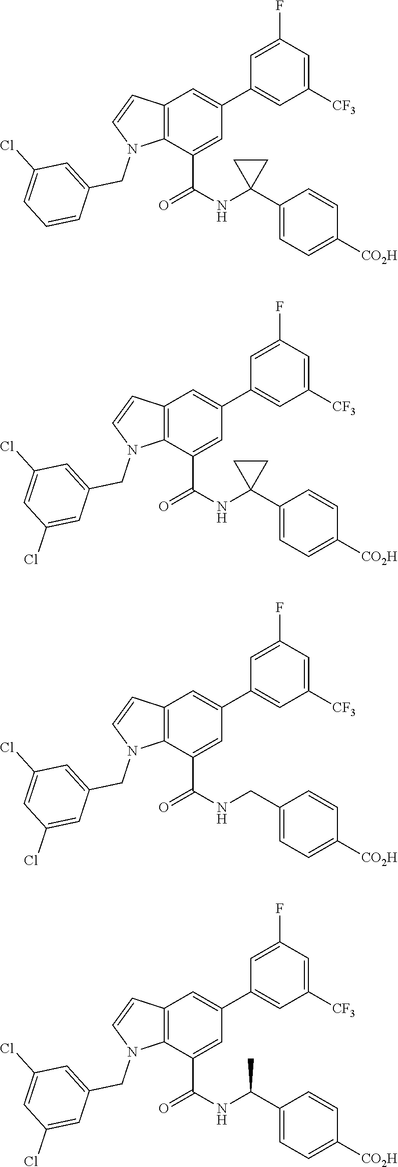 [SMILES: O=C(O)c1ccc(C2(NC(=O)c3cc(-c4cc(F)cc(C(F)(F)F)c4)cc4ccn(Cc5cccc(Cl)c5)c34)CC2)cc1 ; O=C(O)c1ccc(C2(NC(=O)c3cc(-c4cc(F)cc(C(F)(F)F)c4)cc4ccn(Cc5cc(Cl)cc(Cl)c5)c34)CC2)cc1 ; O=C(O)c1ccc(CNC(=O)c2cc(-c3cc(F)cc(C(F)(F)F)c3)cc3ccn(Cc4cc(Cl)cc(Cl)c4)c23)cc1 ; C[C@H](NC(=O)c1cc(-c2cc(F)cc(C(F)(F)F)c2)cc2ccn(Cc3cc(Cl)cc(Cl)c3)c12)c1ccc(C(=O)O)cc1]

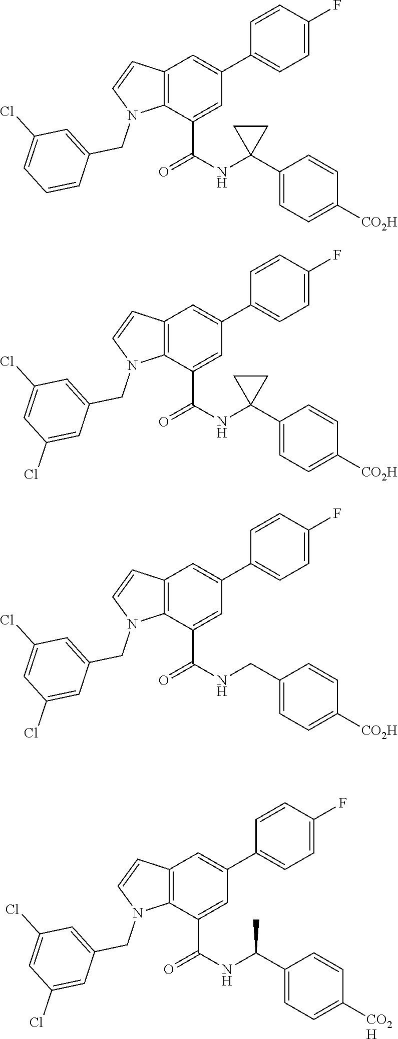

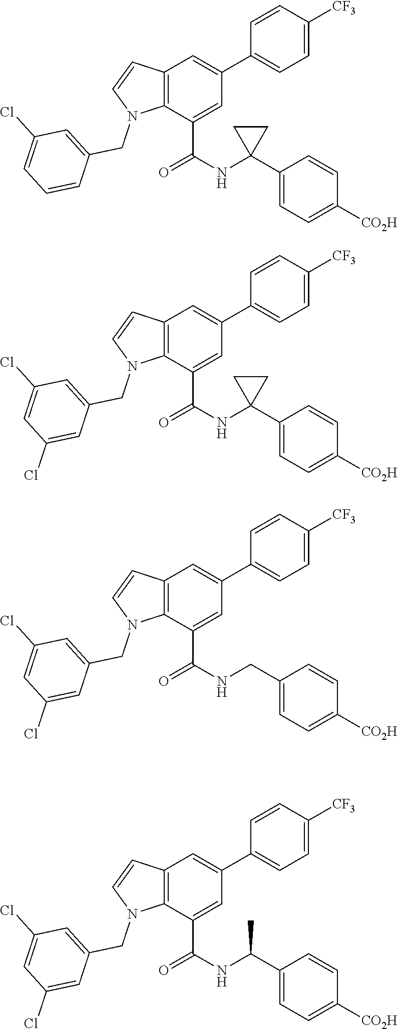

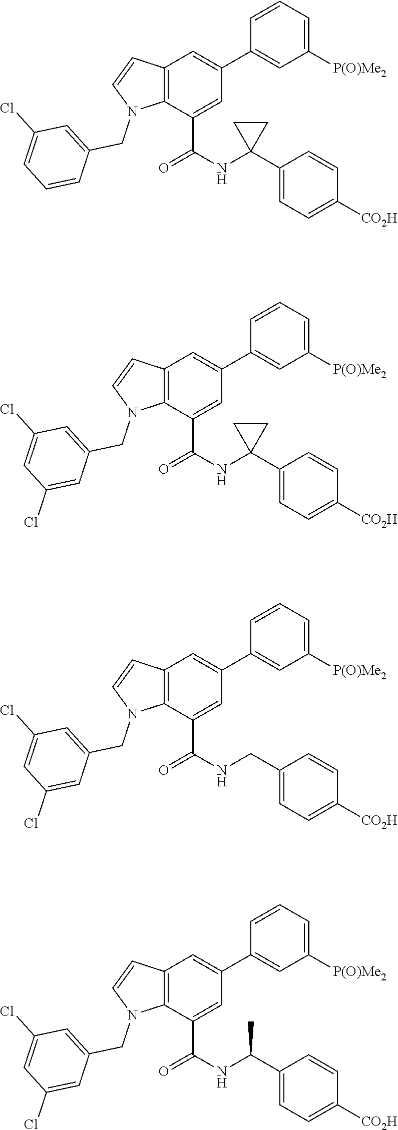

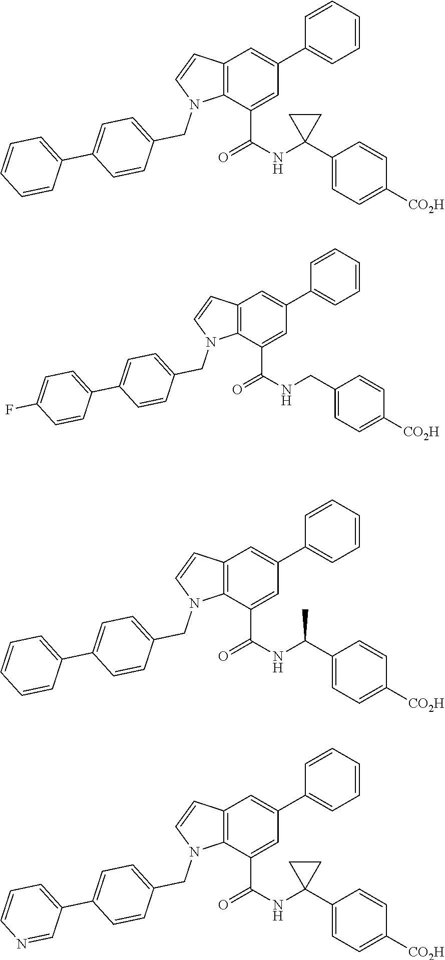 [SMILES: O=C(O)c1ccc(C2(NC(=O)c3cc(-c4ccccc4)cc4ccn(Cc5ccc(-c6ccccc6)cc5)c34)CC2)cc1 ; O=C(O)c1ccc(CNC(=O)c2cc(-c3ccccc3)cc3ccn(Cc4ccc(-c5ccc(F)cc5)cc4)c23)cc1 ; C[C@H](NC(=O)c1cc(-c2ccccc2)cc2ccn(Cc3ccc(-c4ccccc4)cc3)c12)c1ccc(C(=O)O)cc1 ; O=C(O)c1ccc(C2(NC(=O)c3cc(-c4ccccc4)cc4ccn(Cc5ccc(-c6cccnc6)cc5)c34)CC2)cc1]

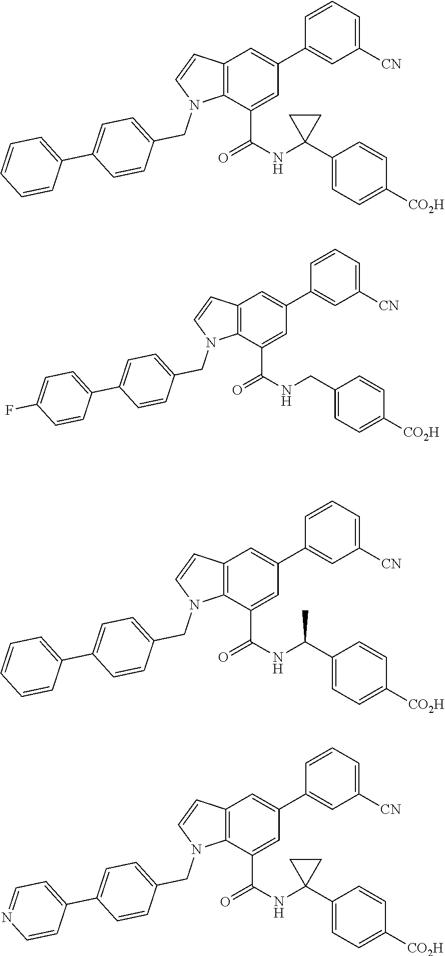 [SMILES: N#Cc1cccc(-c2cc(C(=O)NC3(c4ccc(C(=O)O)cc4)CC3)c3c(ccn3Cc3ccc(-c4ccccc4)cc3)c2)c1 ; N#Cc1cccc(-c2cc(C(=O)NCc3ccc(C(=O)O)cc3)c3c(ccn3Cc3ccc(-c4ccc(F)cc4)cc3)c2)c1 ; C[C@H](NC(=O)c1cc(-c2cccc(C#N)c2)cc2ccn(Cc3ccc(-c4ccccc4)cc3)c12)c1ccc(C(=O)O)cc1 ; N#Cc1cccc(-c2cc(C(=O)NC3(c4ccc(C(=O)O)cc4)CC3)c3c(ccn3Cc3ccc(-c4ccncc4)cc3)c2)c1]

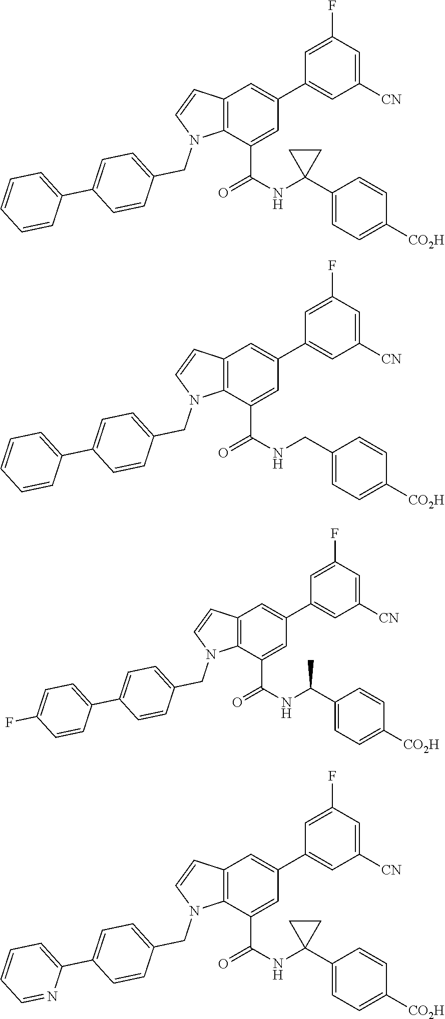

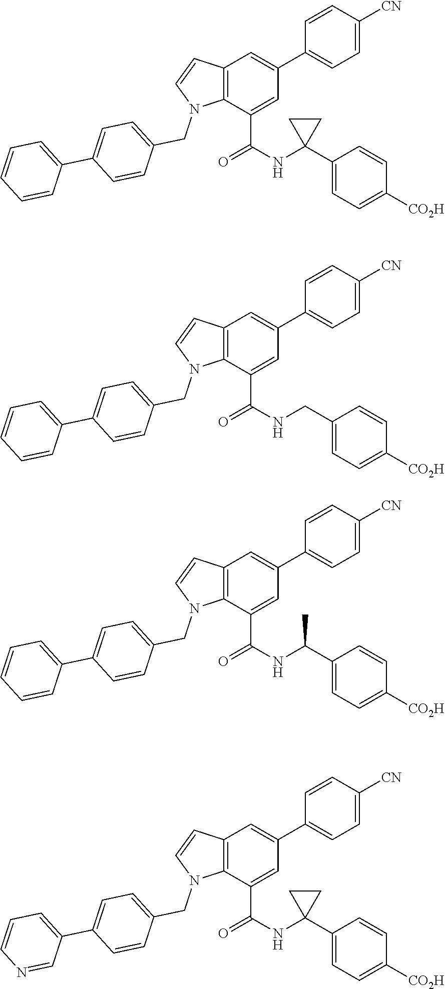 [SMILES: N#Cc1ccc(-c2cc(C(=O)NC3(c4ccc(C(=O)O)cc4)CC3)c3c(ccn3Cc3ccc(-c4ccccc4)cc3)c2)cc1 ; N#Cc1ccc(-c2cc(C(=O)NCc3ccc(C(=O)O)cc3)c3c(ccn3Cc3ccc(-c4ccccc4)cc3)c2)cc1 ; C[C@H](NC(=O)c1cc(-c2ccc(C#N)cc2)cc2ccn(Cc3ccc(-c4ccccc4)cc3)c12)c1ccc(C(=O)O)cc1 ; N#Cc1ccc(-c2cc(C(=O)NC3(c4ccc(C(=O)O)cc4)CC3)c3c(ccn3Cc3ccc(-c4cccnc4)cc3)c2)cc1]

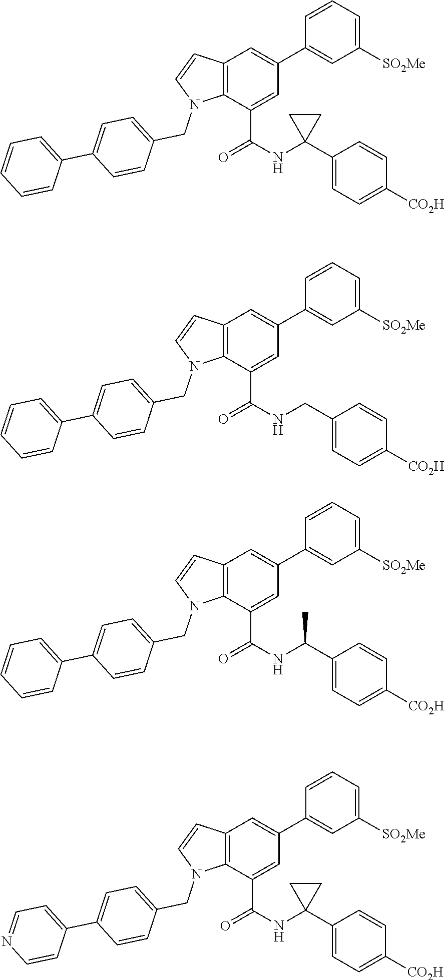 [SMILES: CS(=O)(=O)c1cccc(-c2cc(C(=O)NC3(c4ccc(C(=O)O)cc4)CC3)c3c(ccn3Cc3ccc(-c4ccccc4)cc3)c2)c1 ; CS(=O)(=O)c1cccc(-c2cc(C(=O)NCc3ccc(C(=O)O)cc3)c3c(ccn3Cc3ccc(-c4ccccc4)cc3)c2)c1 ; C[C@H](NC(=O)c1cc(-c2cccc(S(C)(=O)=O)c2)cc2ccn(Cc3ccc(-c4ccccc4)cc3)c12)c1ccc(C(=O)O)cc1 ; CS(=O)(=O)c1cccc(-c2cc(C(=O)NC3(c4ccc(C(=O)O)cc4)CC3)c3c(ccn3Cc3ccc(-c4ccncc4)cc3)c2)c1]

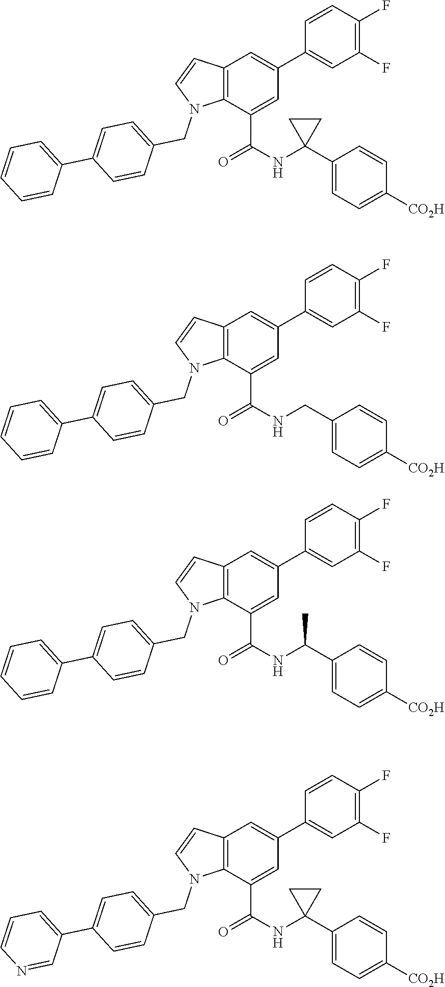 [SMILES: O=C(O)c1ccc(C2(NC(=O)c3cc(-c4ccc(F)c(F)c4)cc4ccn(Cc5ccc(-c6ccccc6)cc5)c34)CC2)cc1 ; O=C(O)c1ccc(CNC(=O)c2cc(-c3ccc(F)c(F)c3)cc3ccn(Cc4ccc(-c5ccccc5)cc4)c23)cc1 ; C[C@H](NC(=O)c1cc(-c2ccc(F)c(F)c2)cc2ccn(Cc3ccc(-c4ccccc4)cc3)c12)c1ccc(C(=O)O)cc1 ; O=C(O)c1ccc(C2(NC(=O)c3cc(-c4ccc(F)c(F)c4)cc4ccn(Cc5ccc(-c6cccnc6)cc5)c34)CC2)cc1]

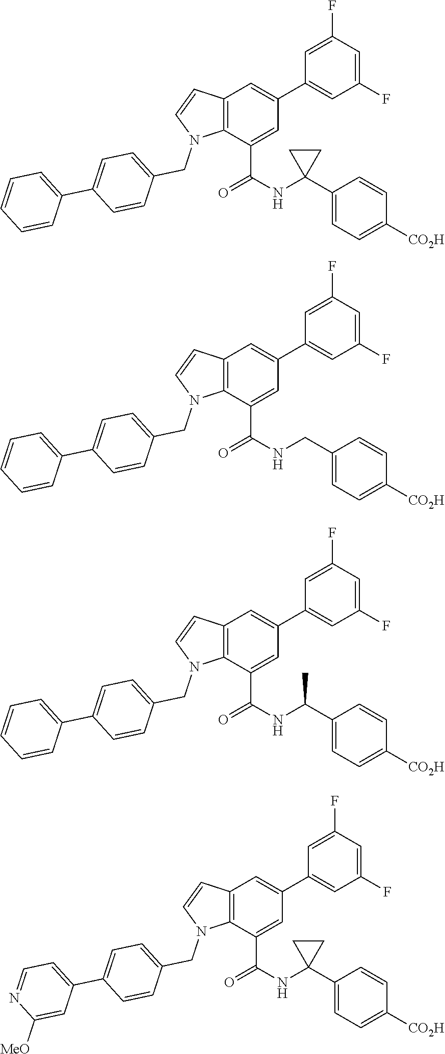

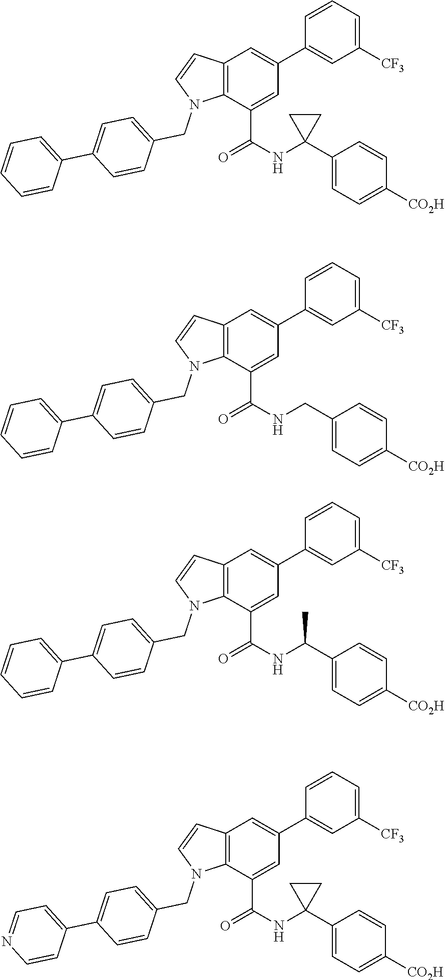 [SMILES: O=C(O)c1ccc(C2(NC(=O)c3cc(-c4cccc(C(F)(F)F)c4)cc4ccn(Cc5ccc(-c6ccccc6)cc5)c34)CC2)cc1 ; O=C(O)c1ccc(CNC(=O)c2cc(-c3cccc(C(F)(F)F)c3)cc3ccn(Cc4ccc(-c5ccccc5)cc4)c23)cc1 ; C[C@H](NC(=O)c1cc(-c2cccc(C(F)(F)F)c2)cc2ccn(Cc3ccc(-c4ccccc4)cc3)c12)c1ccc(C(=O)O)cc1 ; O=C(O)c1ccc(C2(NC(=O)c3cc(-c4cccc(C(F)(F)F)c4)cc4ccn(Cc5ccc(-c6ccncc6)cc5)c34)CC2)cc1]

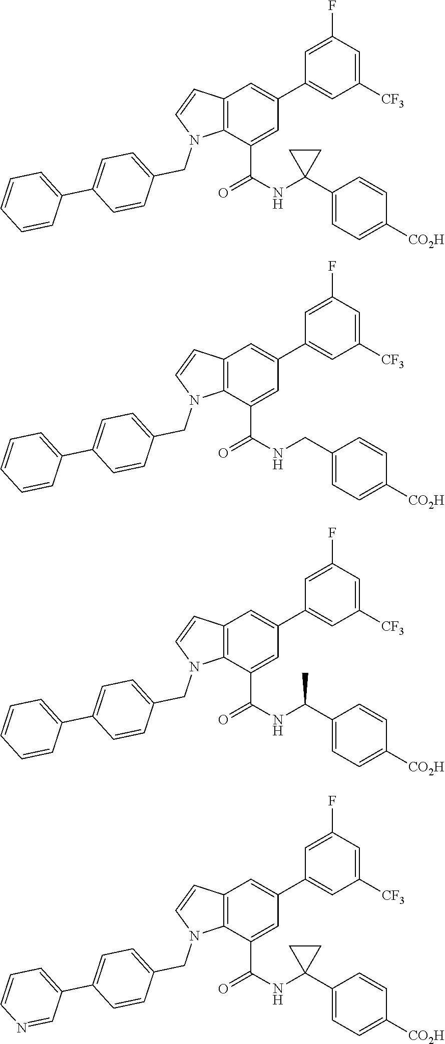

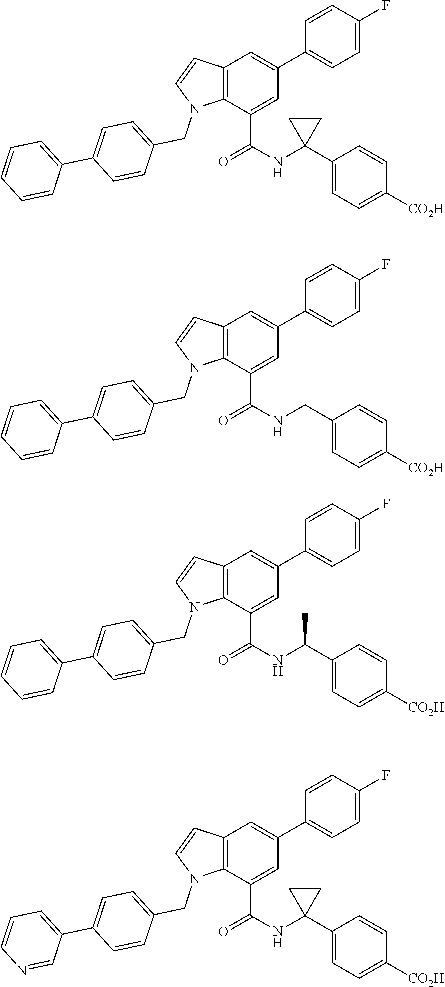 [SMILES: O=C(O)c1ccc(C2(NC(=O)c3cc(-c4ccc(F)cc4)cc4ccn(Cc5ccc(-c6ccccc6)cc5)c34)CC2)cc1 ; O=C(O)c1ccc(CNC(=O)c2cc(-c3ccc(F)cc3)cc3ccn(Cc4ccc(-c5ccccc5)cc4)c23)cc1 ; C[C@H](NC(=O)c1cc(-c2ccc(F)cc2)cc2ccn(Cc3ccc(-c4ccccc4)cc3)c12)c1ccc(C(=O)O)cc1 ; O=C(O)c1ccc(C2(NC(=O)c3cc(-c4ccc(F)cc4)cc4ccn(Cc5ccc(-c6cccnc6)cc5)c34)CC2)cc1]

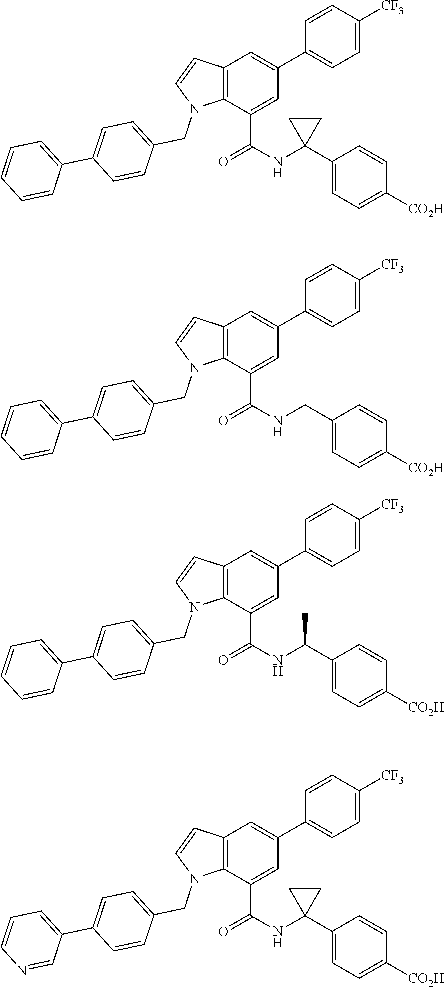 [SMILES: O=C(O)c1ccc(C2(NC(=O)c3cc(-c4ccc(C(F)(F)F)cc4)cc4ccn(Cc5ccc(-c6ccccc6)cc5)c34)CC2)cc1 ; O=C(O)c1ccc(CNC(=O)c2cc(-c3ccc(C(F)(F)F)cc3)cc3ccn(Cc4ccc(-c5ccccc5)cc4)c23)cc1 ; C[C@H](NC(=O)c1cc(-c2ccc(C(F)(F)F)cc2)cc2ccn(Cc3ccc(-c4ccccc4)cc3)c12)c1ccc(C(=O)O)cc1 ; O=C(O)c1ccc(C2(NC(=O)c3cc(-c4ccc(C(F)(F)F)cc4)cc4ccn(Cc5ccc(-c6cccnc6)cc5)c34)CC2)cc1]

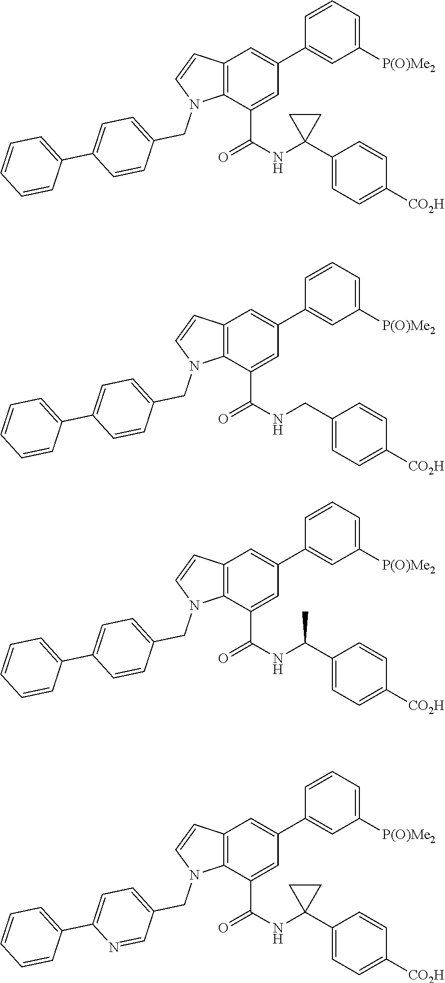

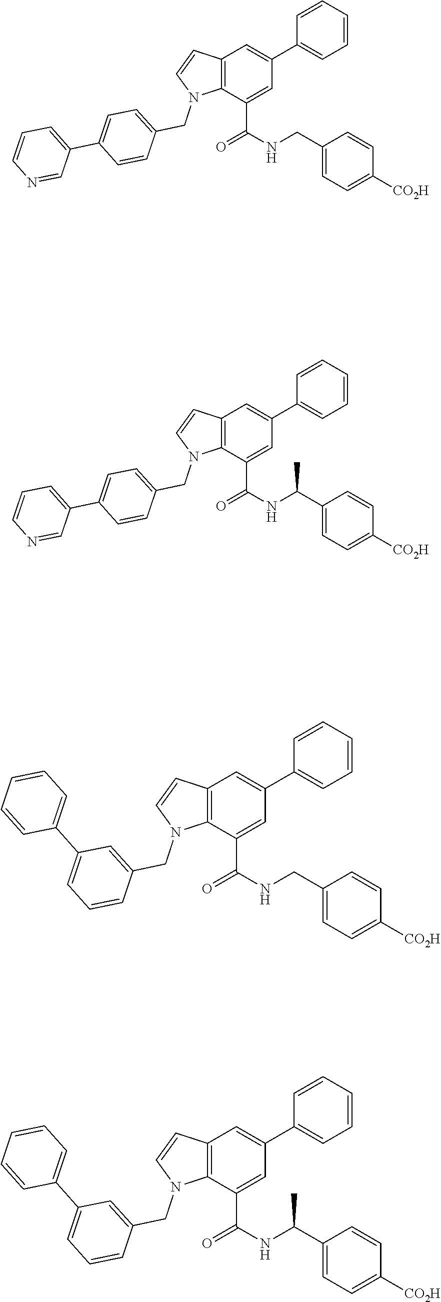 [SMILES: O=C(O)c1ccc(CNC(=O)c2cc(-c3ccccc3)cc3ccn(Cc4ccc(-c5cccnc5)cc4)c23)cc1 ; C[C@H](NC(=O)c1cc(-c2ccccc2)cc2ccn(Cc3ccc(-c4cccnc4)cc3)c12)c1ccc(C(=O)O)cc1 ; O=C(O)c1ccc(CNC(=O)c2cc(-c3ccccc3)cc3ccn(Cc4cccc(-c5ccccc5)c4)c23)cc1 ; C[C@H](NC(=O)c1cc(-c2ccccc2)cc2ccn(Cc3cccc(-c4ccccc4)c3)c12)c1ccc(C(=O)O)cc1]

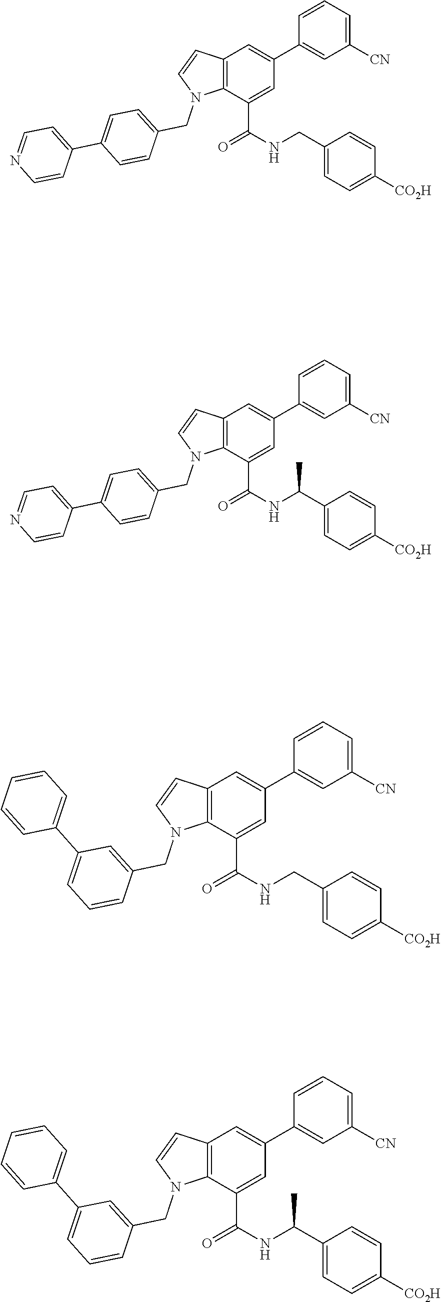 [SMILES: N#Cc1cccc(-c2cc(C(=O)NCc3ccc(C(=O)O)cc3)c3c(ccn3Cc3ccc(-c4ccncc4)cc3)c2)c1 ; C[C@H](NC(=O)c1cc(-c2cccc(C#N)c2)cc2ccn(Cc3ccc(-c4ccncc4)cc3)c12)c1ccc(C(=O)O)cc1 ; N#Cc1cccc(-c2cc(C(=O)NCc3ccc(C(=O)O)cc3)c3c(ccn3Cc3cccc(-c4ccccc4)c3)c2)c1 ; C[C@H](NC(=O)c1cc(-c2cccc(C#N)c2)cc2ccn(Cc3cccc(-c4ccccc4)c3)c12)c1ccc(C(=O)O)cc1]

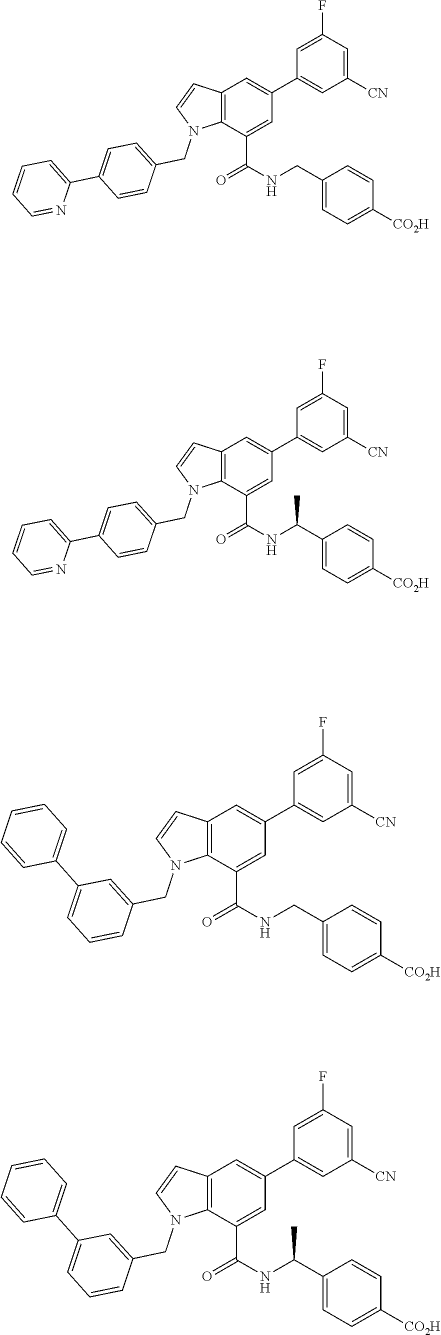 [SMILES: N#Cc1cc(F)cc(-c2cc(C(=O)NCc3ccc(C(=O)O)cc3)c3c(ccn3Cc3ccc(-c4ccccn4)cc3)c2)c1 ; C[C@H](NC(=O)c1cc(-c2cc(F)cc(C#N)c2)cc2ccn(Cc3ccc(-c4ccccn4)cc3)c12)c1ccc(C(=O)O)cc1 ; N#Cc1cc(F)cc(-c2cc(C(=O)NCc3ccc(C(=O)O)cc3)c3c(ccn3Cc3cccc(-c4ccccc4)c3)c2)c1 ; C[C@H](NC(=O)c1cc(-c2cc(F)cc(C#N)c2)cc2ccn(Cc3cccc(-c4ccccc4)c3)c12)c1ccc(C(=O)O)cc1]

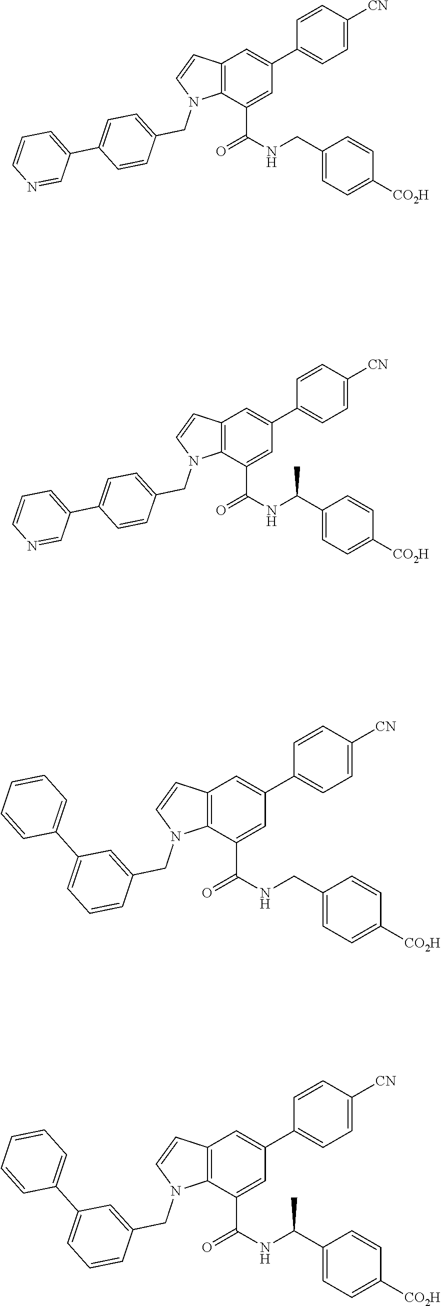 [SMILES: N#Cc1ccc(-c2cc(C(=O)NCc3ccc(C(=O)O)cc3)c3c(ccn3Cc3ccc(-c4cccnc4)cc3)c2)cc1 ; C[C@H](NC(=O)c1cc(-c2ccc(C#N)cc2)cc2ccn(Cc3ccc(-c4cccnc4)cc3)c12)c1ccc(C(=O)O)cc1 ; N#Cc1ccc(-c2cc(C(=O)NCc3ccc(C(=O)O)cc3)c3c(ccn3Cc3cccc(-c4ccccc4)c3)c2)cc1 ; C[C@H](NC(=O)c1cc(-c2ccc(C#N)cc2)cc2ccn(Cc3cccc(-c4ccccc4)c3)c12)c1ccc(C(=O)O)cc1]

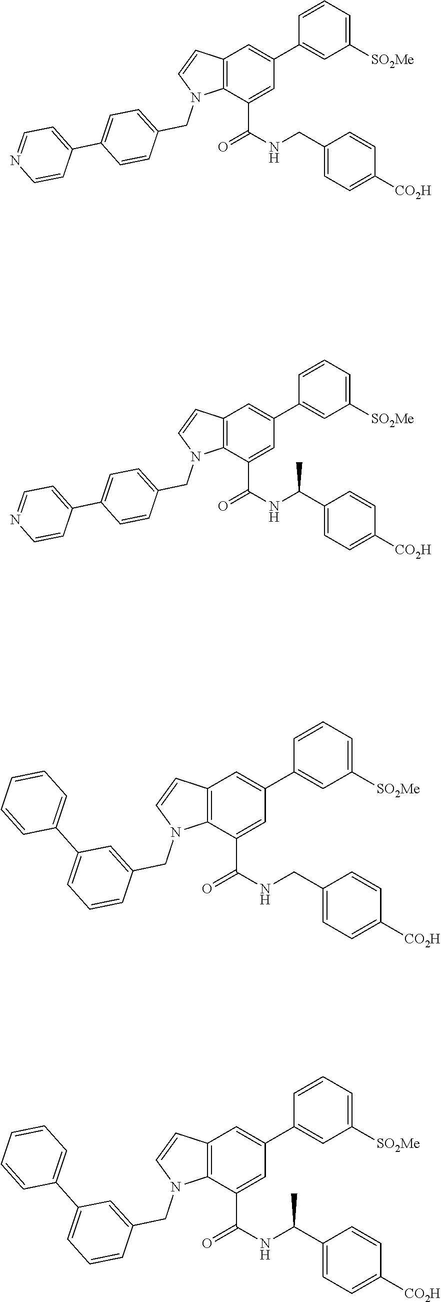 [SMILES: CS(=O)(=O)c1cccc(-c2cc(C(=O)NCc3ccc(C(=O)O)cc3)c3c(ccn3Cc3ccc(-c4ccncc4)cc3)c2)c1 ; C[C@H](NC(=O)c1cc(-c2cccc(S(C)(=O)=O)c2)cc2ccn(Cc3ccc(-c4ccncc4)cc3)c12)c1ccc(C(=O)O)cc1 ; CS(=O)(=O)c1cccc(-c2cc(C(=O)NCc3ccc(C(=O)O)cc3)c3c(ccn3Cc3cccc(-c4ccccc4)c3)c2)c1 ; C[C@H](NC(=O)c1cc(-c2cccc(S(C)(=O)=O)c2)cc2ccn(Cc3cccc(-c4ccccc4)c3)c12)c1ccc(C(=O)O)cc1]

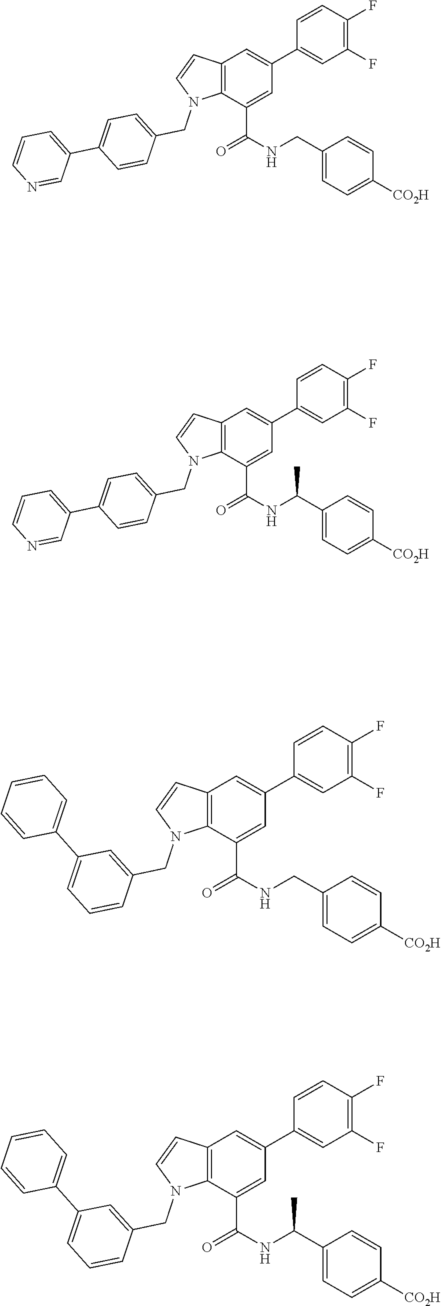 [SMILES: O=C(O)c1ccc(CNC(=O)c2cc(-c3ccc(F)c(F)c3)cc3ccn(Cc4ccc(-c5cccnc5)cc4)c23)cc1 ; C[C@H](NC(=O)c1cc(-c2ccc(F)c(F)c2)cc2ccn(Cc3ccc(-c4cccnc4)cc3)c12)c1ccc(C(=O)O)cc1 ; O=C(O)c1ccc(CNC(=O)c2cc(-c3ccc(F)c(F)c3)cc3ccn(Cc4cccc(-c5ccccc5)c4)c23)cc1 ; C[C@H](NC(=O)c1cc(-c2ccc(F)c(F)c2)cc2ccn(Cc3cccc(-c4ccccc4)c3)c12)c1ccc(C(=O)O)cc1]

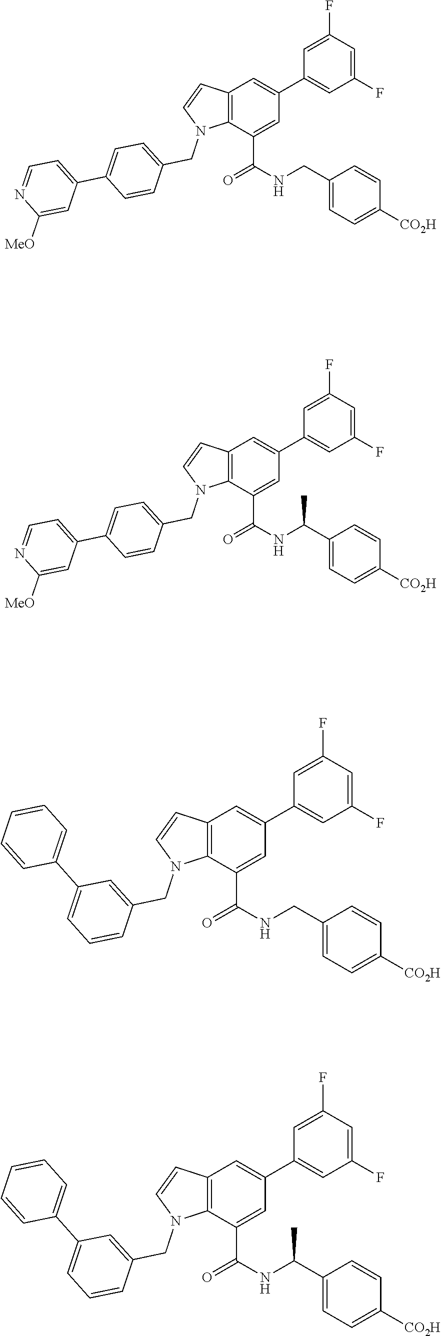 [SMILES: COc1cc(-c2ccc(Cn3ccc4cc(-c5cc(F)cc(F)c5)cc(C(=O)NCc5ccc(C(=O)O)cc5)c43)cc2)ccn1 ; COc1cc(-c2ccc(Cn3ccc4cc(-c5cc(F)cc(F)c5)cc(C(=O)N[C@@H](C)c5ccc(C(=O)O)cc5)c43)cc2)ccn1 ; O=C(O)c1ccc(CNC(=O)c2cc(-c3cc(F)cc(F)c3)cc3ccn(Cc4cccc(-c5ccccc5)c4)c23)cc1 ; C[C@H](NC(=O)c1cc(-c2cc(F)cc(F)c2)cc2ccn(Cc3cccc(-c4ccccc4)c3)c12)c1ccc(C(=O)O)cc1]

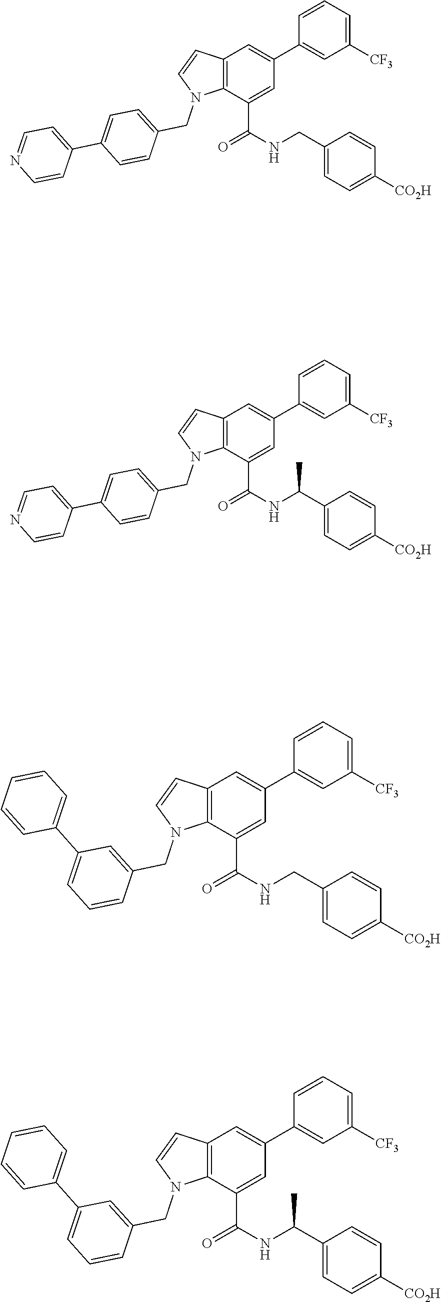 [SMILES: O=C(O)c1ccc(CNC(=O)c2cc(-c3cccc(C(F)(F)F)c3)cc3ccn(Cc4ccc(-c5ccncc5)cc4)c23)cc1 ; C[C@H](NC(=O)c1cc(-c2cccc(C(F)(F)F)c2)cc2ccn(Cc3ccc(-c4ccncc4)cc3)c12)c1ccc(C(=O)O)cc1 ; O=C(O)c1ccc(CNC(=O)c2cc(-c3cccc(C(F)(F)F)c3)cc3ccn(Cc4cccc(-c5ccccc5)c4)c23)cc1 ; C[C@H](NC(=O)c1cc(-c2cccc(C(F)(F)F)c2)cc2ccn(Cc3cccc(-c4ccccc4)c3)c12)c1ccc(C(=O)O)cc1]

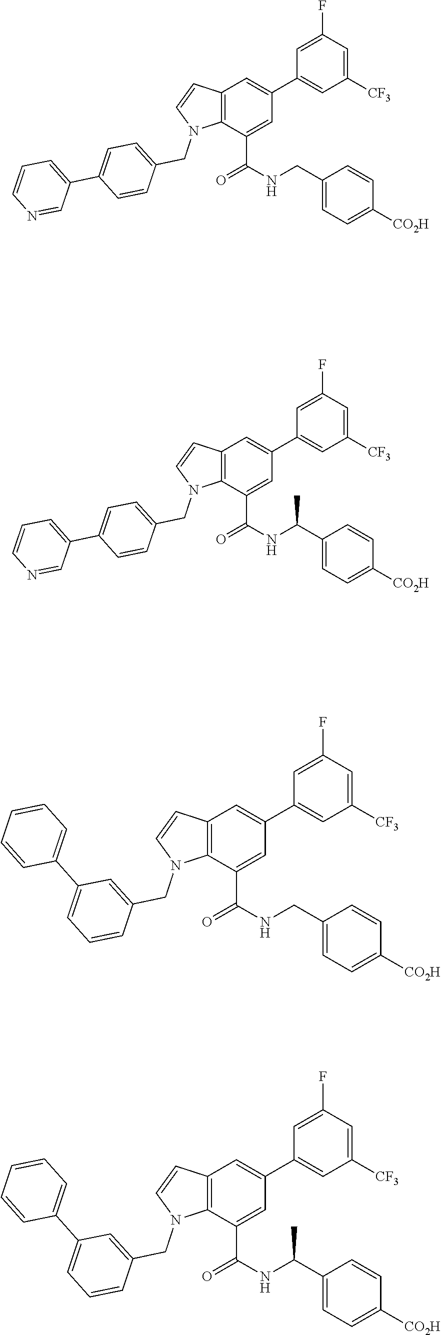 [SMILES: O=C(O)c1ccc(CNC(=O)c2cc(-c3cc(F)cc(C(F)(F)F)c3)cc3ccn(Cc4ccc(-c5cccnc5)cc4)c23)cc1 ; C[C@H](NC(=O)c1cc(-c2cc(F)cc(C(F)(F)F)c2)cc2ccn(Cc3ccc(-c4cccnc4)cc3)c12)c1ccc(C(=O)O)cc1 ; O=C(O)c1ccc(CNC(=O)c2cc(-c3cc(F)cc(C(F)(F)F)c3)cc3ccn(Cc4cccc(-c5ccccc5)c4)c23)cc1 ; C[C@H](NC(=O)c1cc(-c2cc(F)cc(C(F)(F)F)c2)cc2ccn(Cc3cccc(-c4ccccc4)c3)c12)c1ccc(C(=O)O)cc1]

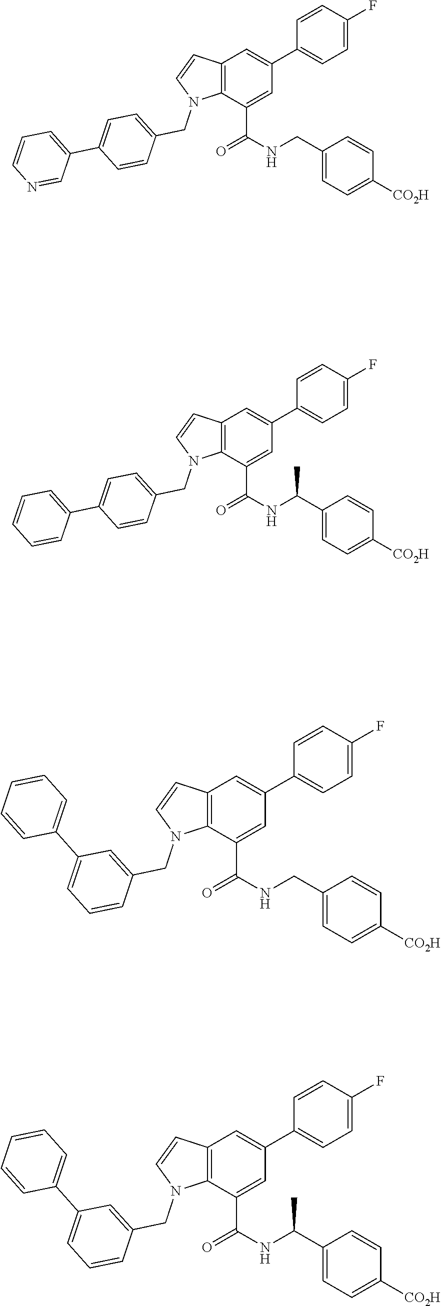 [SMILES: O=C(O)c1ccc(CNC(=O)c2cc(-c3ccc(F)cc3)cc3ccn(Cc4ccc(-c5cccnc5)cc4)c23)cc1 ; C[C@H](NC(=O)c1cc(-c2ccc(F)cc2)cc2ccn(Cc3ccc(-c4ccccc4)cc3)c12)c1ccc(C(=O)O)cc1 ; O=C(O)c1ccc(CNC(=O)c2cc(-c3ccc(F)cc3)cc3ccn(Cc4cccc(-c5ccccc5)c4)c23)cc1 ; C[C@H](NC(=O)c1cc(-c2ccc(F)cc2)cc2ccn(Cc3cccc(-c4ccccc4)c3)c12)c1ccc(C(=O)O)cc1]

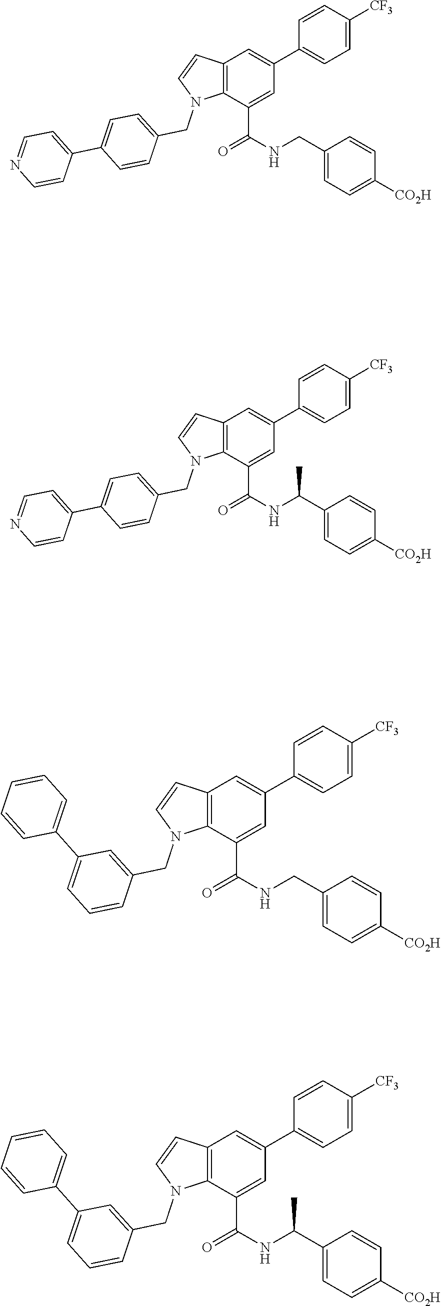 [SMILES: O=C(O)c1ccc(CNC(=O)c2cc(-c3ccc(C(F)(F)F)cc3)cc3ccn(Cc4ccc(-c5ccncc5)cc4)c23)cc1 ; C[C@H](NC(=O)c1cc(-c2ccc(C(F)(F)F)cc2)cc2ccn(Cc3ccc(-c4ccncc4)cc3)c12)c1ccc(C(=O)O)cc1 ; O=C(O)c1ccc(CNC(=O)c2cc(-c3ccc(C(F)(F)F)cc3)cc3ccn(Cc4cccc(-c5ccccc5)c4)c23)cc1 ; C[C@H](NC(=O)c1cc(-c2ccc(C(F)(F)F)cc2)cc2ccn(Cc3cccc(-c4ccccc4)c3)c12)c1ccc(C(=O)O)cc1]

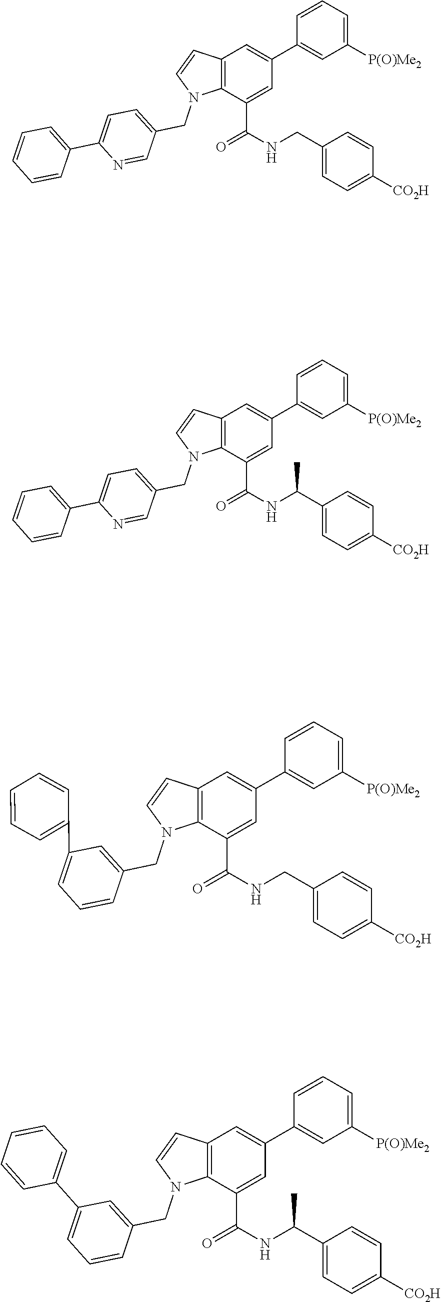

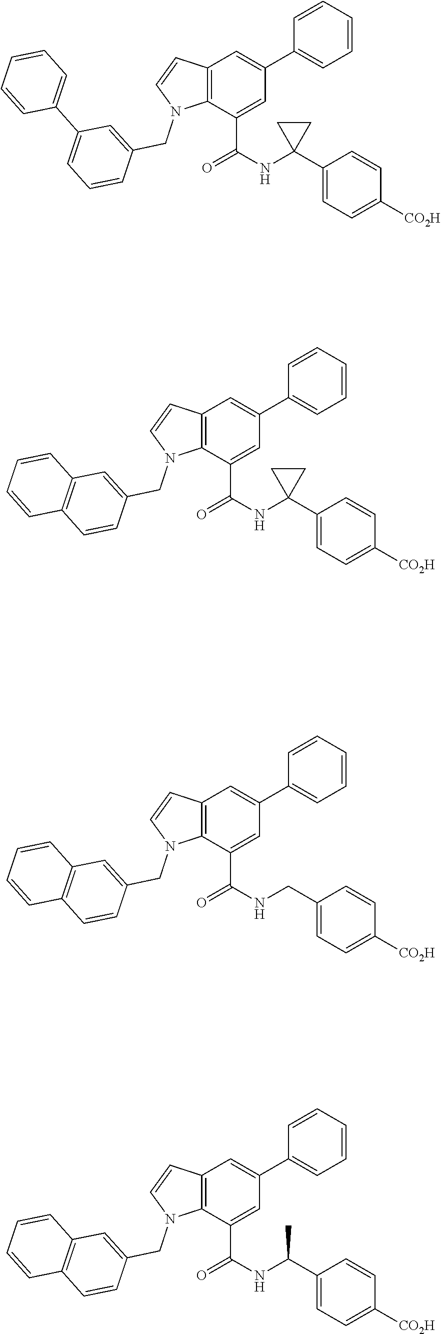 [SMILES: O=C(O)c1ccc(C2(NC(=O)c3cc(-c4ccccc4)cc4ccn(Cc5cccc(-c6ccccc6)c5)c34)CC2)cc1 ; O=C(O)c1ccc(C2(NC(=O)c3cc(-c4ccccc4)cc4ccn(Cc5ccc6ccccc6c5)c34)CC2)cc1 ; O=C(O)c1ccc(CNC(=O)c2cc(-c3ccccc3)cc3ccn(Cc4ccc5ccccc5c4)c23)cc1 ; C[C@H](NC(=O)c1cc(-c2ccccc2)cc2ccn(Cc3ccc4ccccc4c3)c12)c1ccc(C(=O)O)cc1]

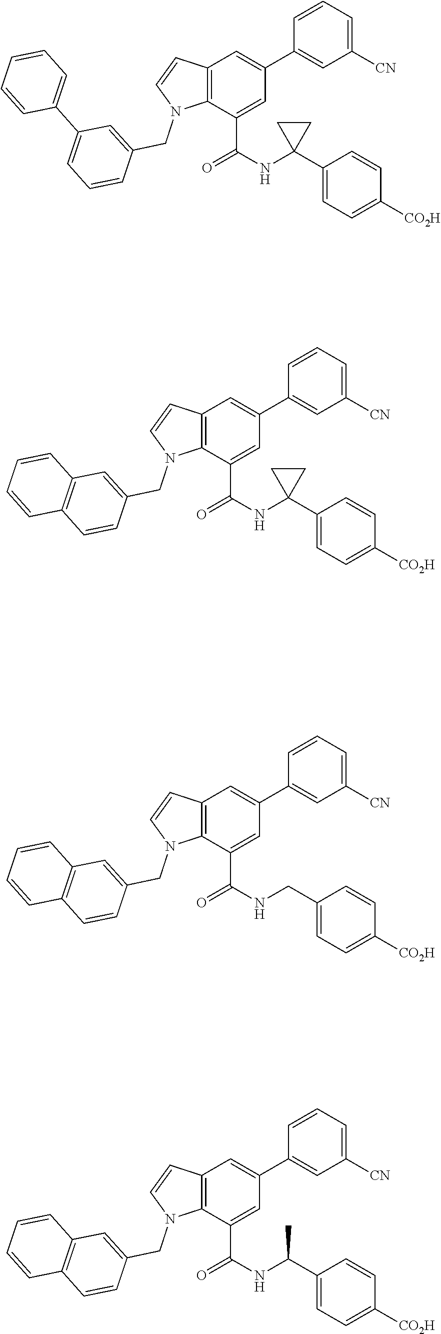 [SMILES: N#Cc1cccc(-c2cc(C(=O)NC3(c4ccc(C(=O)O)cc4)CC3)c3c(ccn3Cc3cccc(-c4ccccc4)c3)c2)c1 ; N#Cc1cccc(-c2cc(C(=O)NC3(c4ccc(C(=O)O)cc4)CC3)c3c(ccn3Cc3ccc4ccccc4c3)c2)c1 ; N#Cc1cccc(-c2cc(C(=O)NCc3ccc(C(=O)O)cc3)c3c(ccn3Cc3ccc4ccccc4c3)c2)c1 ; C[C@H](NC(=O)c1cc(-c2cccc(C#N)c2)cc2ccn(Cc3ccc4ccccc4c3)c12)c1ccc(C(=O)O)cc1]

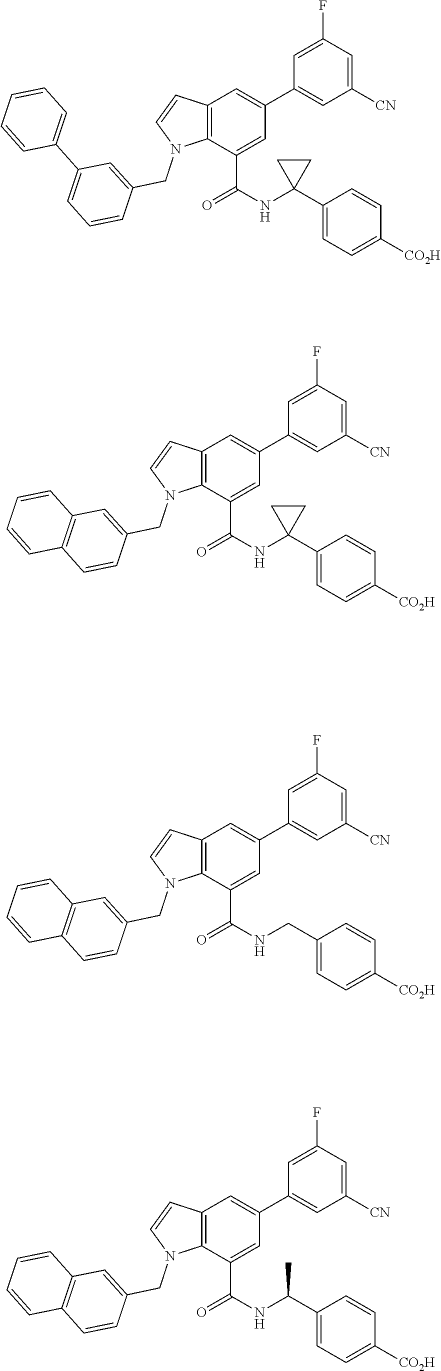 [SMILES: N#Cc1cc(F)cc(-c2cc(C(=O)NC3(c4ccc(C(=O)O)cc4)CC3)c3c(ccn3Cc3cccc(-c4ccccc4)c3)c2)c1 ; N#Cc1cc(F)cc(-c2cc(C(=O)NC3(c4ccc(C(=O)O)cc4)CC3)c3c(ccn3Cc3ccc4ccccc4c3)c2)c1 ; N#Cc1cc(F)cc(-c2cc(C(=O)NCc3ccc(C(=O)O)cc3)c3c(ccn3Cc3ccc4ccccc4c3)c2)c1 ; C[C@H](NC(=O)c1cc(-c2cc(F)cc(C#N)c2)cc2ccn(Cc3ccc4ccccc4c3)c12)c1ccc(C(=O)O)cc1]

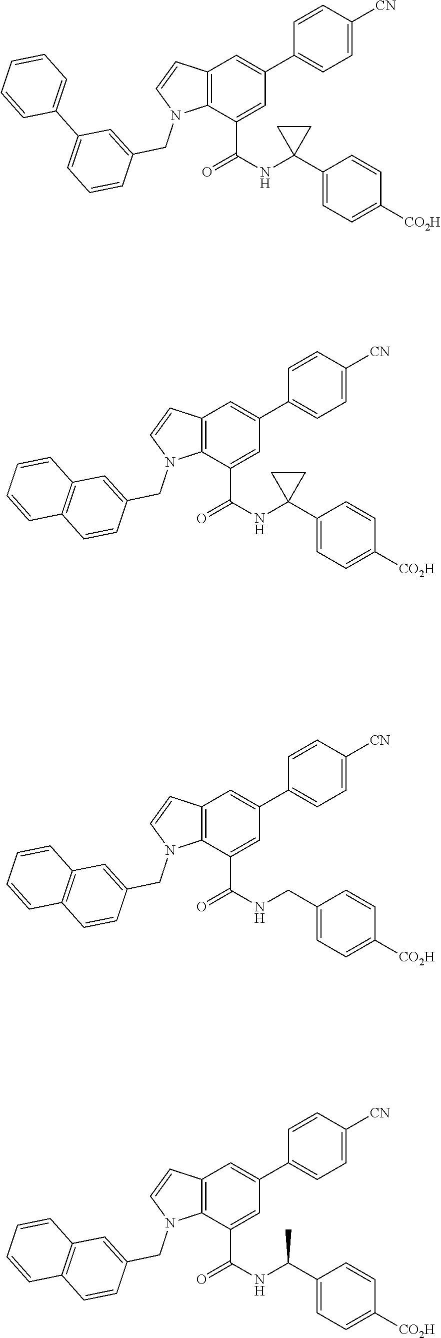 [SMILES: N#Cc1ccc(-c2cc(C(=O)NC3(c4ccc(C(=O)O)cc4)CC3)c3c(ccn3Cc3cccc(-c4ccccc4)c3)c2)cc1 ; N#Cc1ccc(-c2cc(C(=O)NC3(c4ccc(C(=O)O)cc4)CC3)c3c(ccn3Cc3ccc4ccccc4c3)c2)cc1 ; N#Cc1ccc(-c2cc(C(=O)NCc3ccc(C(=O)O)cc3)c3c(ccn3Cc3ccc4ccccc4c3)c2)cc1 ; C[C@H](NC(=O)c1cc(-c2ccc(C#N)cc2)cc2ccn(Cc3ccc4ccccc4c3)c12)c1ccc(C(=O)O)cc1]

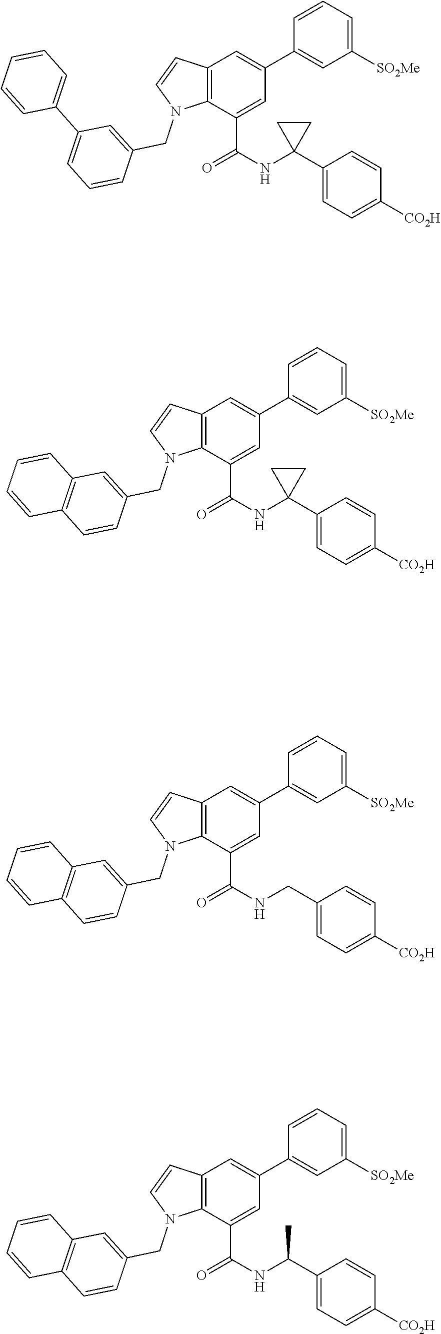 [SMILES: CS(=O)(=O)c1cccc(-c2cc(C(=O)NC3(c4ccc(C(=O)O)cc4)CC3)c3c(ccn3Cc3cccc(-c4ccccc4)c3)c2)c1 ; CS(=O)(=O)c1cccc(-c2cc(C(=O)NC3(c4ccc(C(=O)O)cc4)CC3)c3c(ccn3Cc3ccc4ccccc4c3)c2)c1 ; CS(=O)(=O)c1cccc(-c2cc(C(=O)NCc3ccc(C(=O)O)cc3)c3c(ccn3Cc3ccc4ccccc4c3)c2)c1 ; C[C@H](NC(=O)c1cc(-c2cccc(S(C)(=O)=O)c2)cc2ccn(Cc3ccc4ccccc4c3)c12)c1ccc(C(=O)O)cc1]

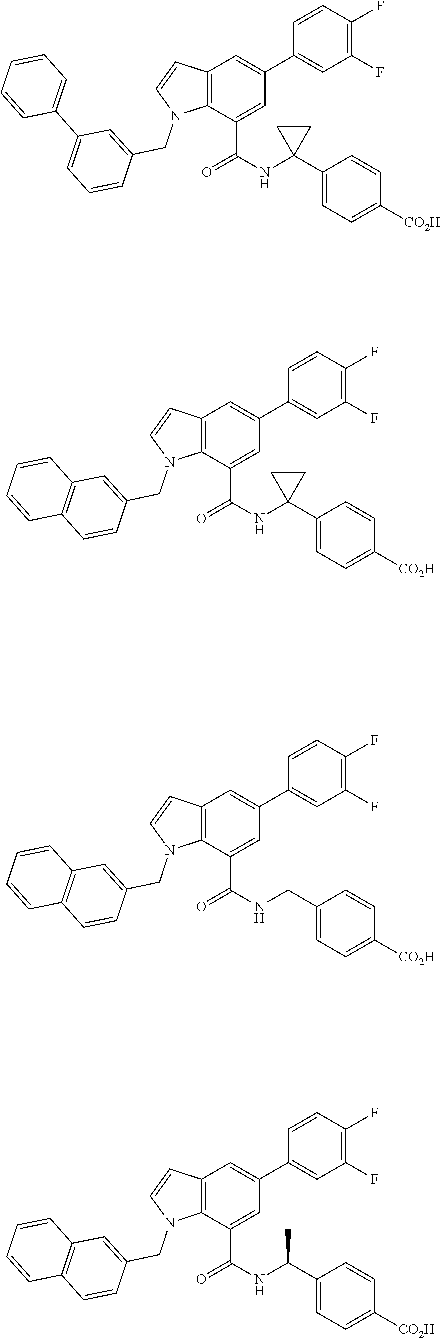 [SMILES: O=C(O)c1ccc(C2(NC(=O)c3cc(-c4ccc(F)c(F)c4)cc4ccn(Cc5cccc(-c6ccccc6)c5)c34)CC2)cc1 ; O=C(O)c1ccc(C2(NC(=O)c3cc(-c4ccc(F)c(F)c4)cc4ccn(Cc5ccc6ccccc6c5)c34)CC2)cc1 ; O=C(O)c1ccc(CNC(=O)c2cc(-c3ccc(F)c(F)c3)cc3ccn(Cc4ccc5ccccc5c4)c23)cc1 ; C[C@H](NC(=O)c1cc(-c2ccc(F)c(F)c2)cc2ccn(Cc3ccc4ccccc4c3)c12)c1ccc(C(=O)O)cc1]

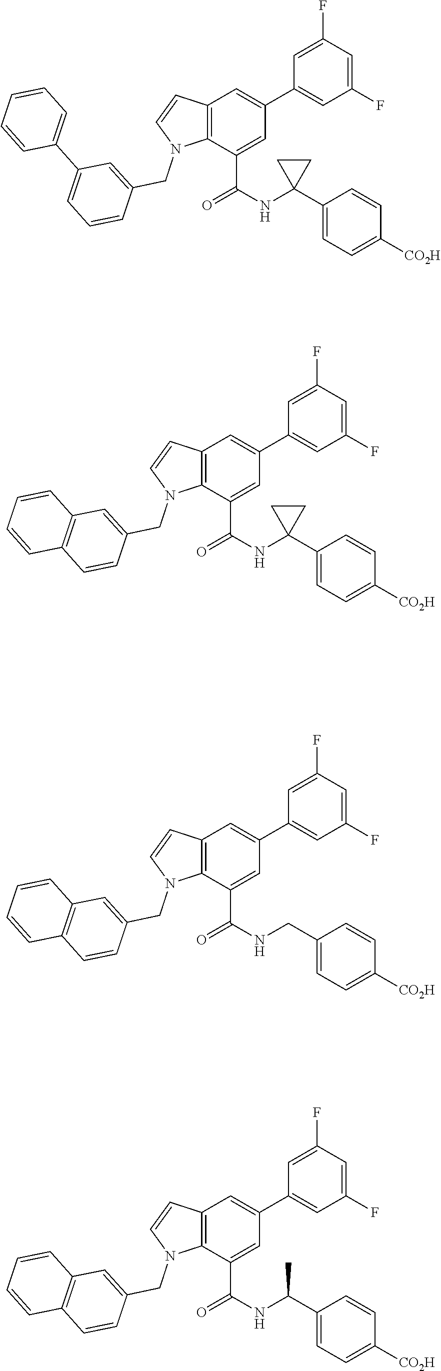 [SMILES: O=C(O)c1ccc(C2(NC(=O)c3cc(-c4cc(F)cc(F)c4)cc4ccn(Cc5cccc(-c6ccccc6)c5)c34)CC2)cc1 ; O=C(O)c1ccc(C2(NC(=O)c3cc(-c4cc(F)cc(F)c4)cc4ccn(Cc5ccc6ccccc6c5)c34)CC2)cc1 ; O=C(O)c1ccc(CNC(=O)c2cc(-c3cc(F)cc(F)c3)cc3ccn(Cc4ccc5ccccc5c4)c23)cc1 ; C[C@H](NC(=O)c1cc(-c2cc(F)cc(F)c2)cc2ccn(Cc3ccc4ccccc4c3)c12)c1ccc(C(=O)O)cc1]

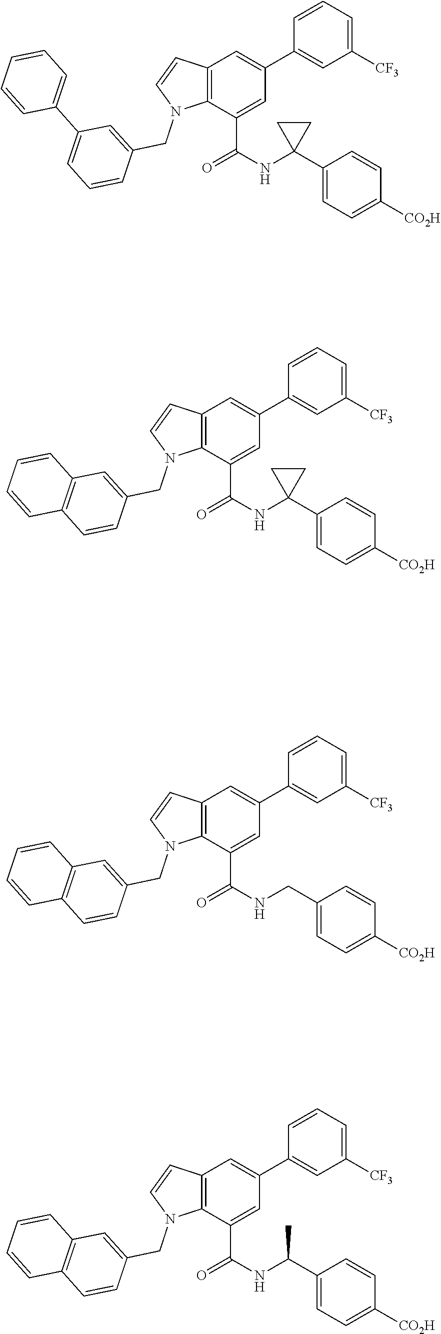 [SMILES: O=C(O)c1ccc(C2(NC(=O)c3cc(-c4cccc(C(F)(F)F)c4)cc4ccn(Cc5cccc(-c6ccccc6)c5)c34)CC2)cc1 ; O=C(O)c1ccc(C2(NC(=O)c3cc(-c4cccc(C(F)(F)F)c4)cc4ccn(Cc5ccc6ccccc6c5)c34)CC2)cc1 ; O=C(O)c1ccc(CNC(=O)c2cc(-c3cccc(C(F)(F)F)c3)cc3ccn(Cc4ccc5ccccc5c4)c23)cc1 ; C[C@H](NC(=O)c1cc(-c2cccc(C(F)(F)F)c2)cc2ccn(Cc3ccc4ccccc4c3)c12)c1ccc(C(=O)O)cc1]

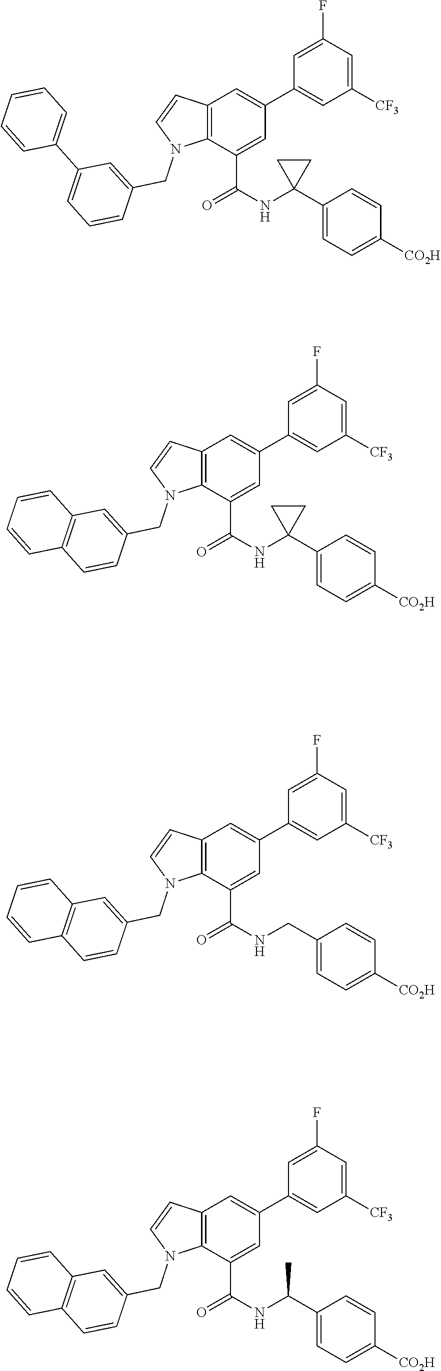 [SMILES: O=C(O)c1ccc(C2(NC(=O)c3cc(-c4cc(F)cc(C(F)(F)F)c4)cc4ccn(Cc5cccc(-c6ccccc6)c5)c34)CC2)cc1 ; O=C(O)c1ccc(C2(NC(=O)c3cc(-c4cc(F)cc(C(F)(F)F)c4)cc4ccn(Cc5ccc6ccccc6c5)c34)CC2)cc1 ; O=C(O)c1ccc(CNC(=O)c2cc(-c3cc(F)cc(C(F)(F)F)c3)cc3ccn(Cc4ccc5ccccc5c4)c23)cc1 ; C[C@H](NC(=O)c1cc(-c2cc(F)cc(C(F)(F)F)c2)cc2ccn(Cc3ccc4ccccc4c3)c12)c1ccc(C(=O)O)cc1]

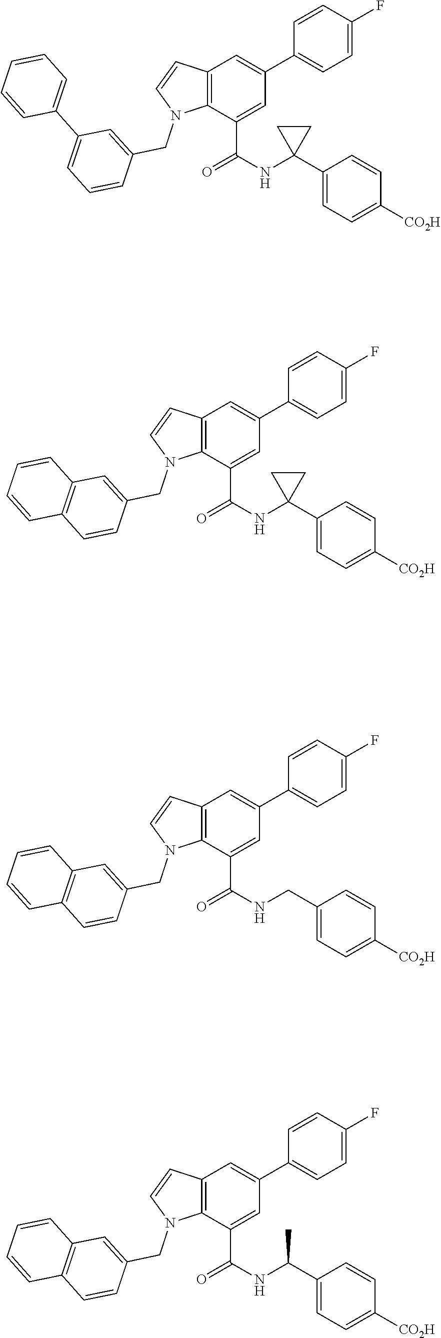 [SMILES: O=C(O)c1ccc(C2(NC(=O)c3cc(-c4ccc(F)cc4)cc4ccn(Cc5cccc(-c6ccccc6)c5)c34)CC2)cc1 ; O=C(O)c1ccc(C2(NC(=O)c3cc(-c4ccc(F)cc4)cc4ccn(Cc5ccc6ccccc6c5)c34)CC2)cc1 ; O=C(O)c1ccc(CNC(=O)c2cc(-c3ccc(F)cc3)cc3ccn(Cc4ccc5ccccc5c4)c23)cc1 ; C[C@H](NC(=O)c1cc(-c2ccc(F)cc2)cc2ccn(Cc3ccc4ccccc4c3)c12)c1ccc(C(=O)O)cc1]

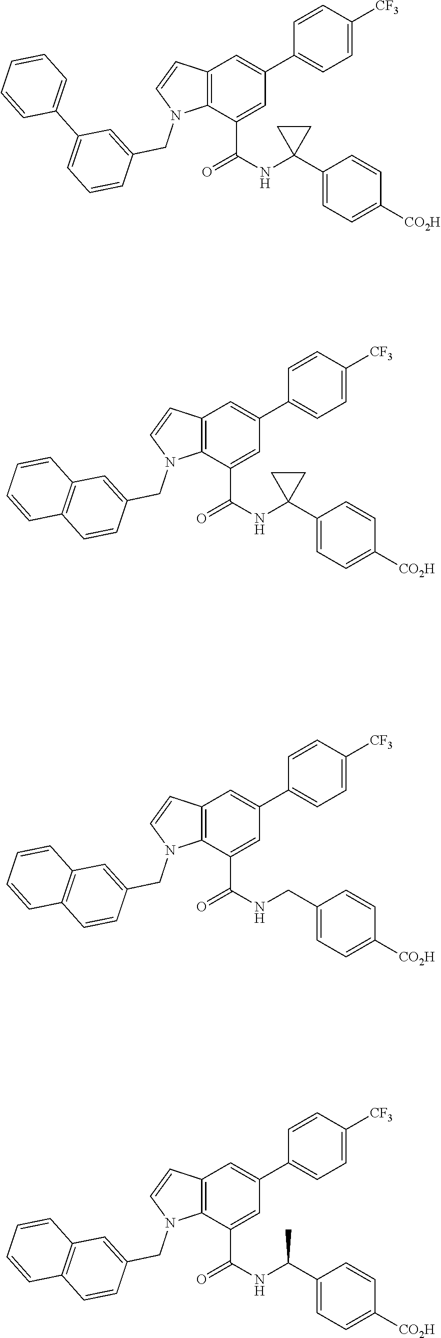 [SMILES: O=C(O)c1ccc(C2(NC(=O)c3cc(-c4ccc(C(F)(F)F)cc4)cc4ccn(Cc5cccc(-c6ccccc6)c5)c34)CC2)cc1 ; O=C(O)c1ccc(C2(NC(=O)c3cc(-c4ccc(C(F)(F)F)cc4)cc4ccn(Cc5ccc6ccccc6c5)c34)CC2)cc1 ; O=C(O)c1ccc(CNC(=O)c2cc(-c3ccc(C(F)(F)F)cc3)cc3ccn(Cc4ccc5ccccc5c4)c23)cc1 ; C[C@H](NC(=O)c1cc(-c2ccc(C(F)(F)F)cc2)cc2ccn(Cc3ccc4ccccc4c3)c12)c1ccc(C(=O)O)cc1]

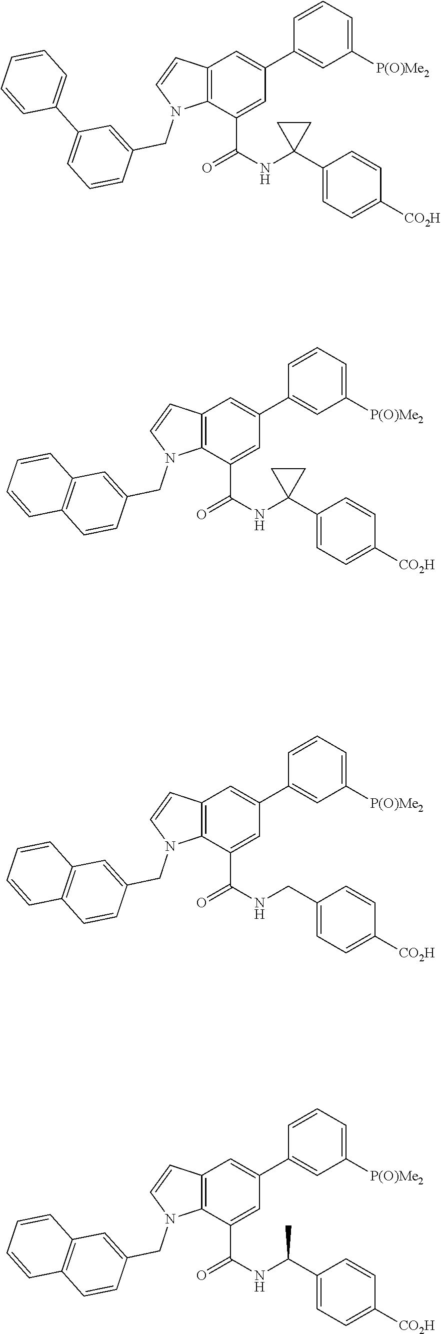

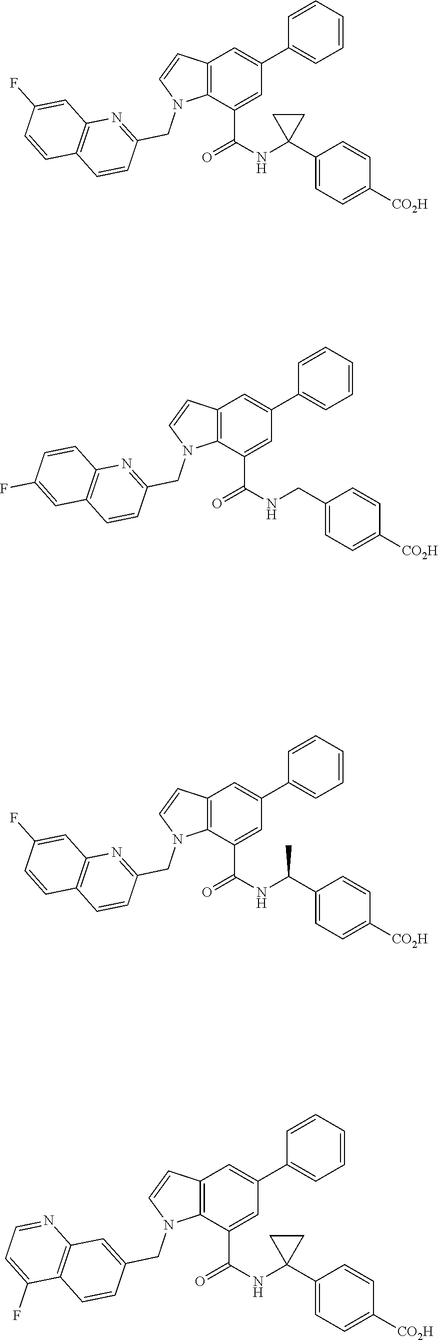 [SMILES: O=C(O)c1ccc(C2(NC(=O)c3cc(-c4ccccc4)cc4ccn(Cc5ccc6ccc(F)cc6n5)c34)CC2)cc1 ; O=C(O)c1ccc(CNC(=O)c2cc(-c3ccccc3)cc3ccn(Cc4ccc5cc(F)ccc5n4)c23)cc1 ; C[C@H](NC(=O)c1cc(-c2ccccc2)cc2ccn(Cc3ccc4ccc(F)cc4n3)c12)c1ccc(C(=O)O)cc1 ; O=C(O)c1ccc(C2(NC(=O)c3cc(-c4ccccc4)cc4ccn(Cc5ccc6c(F)ccnc6c5)c34)CC2)cc1]

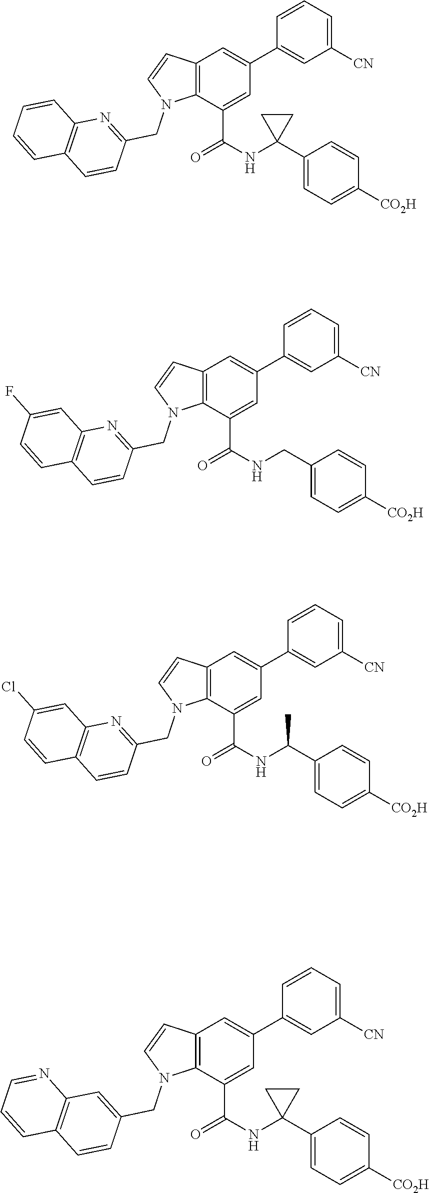 [SMILES: N#Cc1cccc(-c2cc(C(=O)NC3(c4ccc(C(=O)O)cc4)CC3)c3c(ccn3Cc3ccc4ccccc4n3)c2)c1 ; N#Cc1cccc(-c2cc(C(=O)NCc3ccc(C(=O)O)cc3)c3c(ccn3Cc3ccc4ccc(F)cc4n3)c2)c1 ; C[C@H](NC(=O)c1cc(-c2cccc(C#N)c2)cc2ccn(Cc3ccc4ccc(Cl)cc4n3)c12)c1ccc(C(=O)O)cc1 ; N#Cc1cccc(-c2cc(C(=O)NC3(c4ccc(C(=O)O)cc4)CC3)c3c(ccn3Cc3ccc4cccnc4c3)c2)c1]

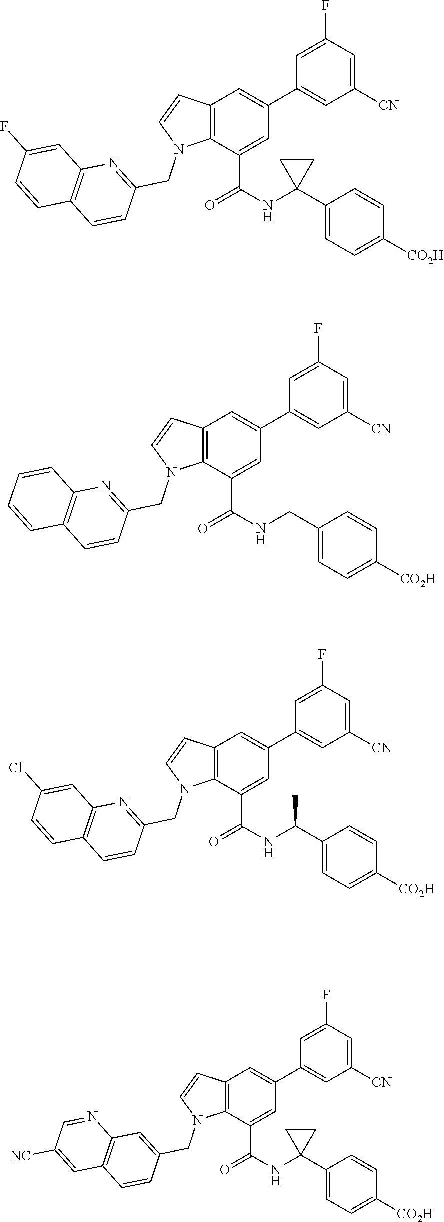 [SMILES: N#Cc1cc(F)cc(-c2cc(C(=O)NC3(c4ccc(C(=O)O)cc4)CC3)c3c(ccn3Cc3ccc4ccc(F)cc4n3)c2)c1 ; N#Cc1cc(F)cc(-c2cc(C(=O)NCc3ccc(C(=O)O)cc3)c3c(ccn3Cc3ccc4ccccc4n3)c2)c1 ; C[C@H](NC(=O)c1cc(-c2cc(F)cc(C#N)c2)cc2ccn(Cc3ccc4ccc(Cl)cc4n3)c12)c1ccc(C(=O)O)cc1 ; N#Cc1cc(F)cc(-c2cc(C(=O)NC3(c4ccc(C(=O)O)cc4)CC3)c3c(ccn3Cc3ccc4cc(C#N)cnc4c3)c2)c1]

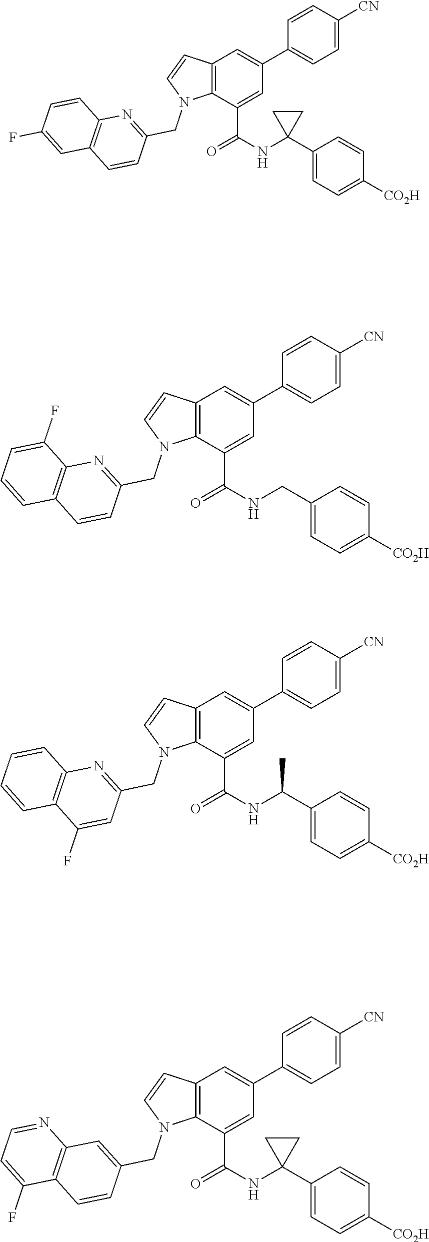 [SMILES: N#Cc1ccc(-c2cc(C(=O)NC3(c4ccc(C(=O)O)cc4)CC3)c3c(ccn3Cc3ccc4cc(F)ccc4n3)c2)cc1 ; N#Cc1ccc(-c2cc(C(=O)NCc3ccc(C(=O)O)cc3)c3c(ccn3Cc3ccc4cccc(F)c4n3)c2)cc1 ; C[C@H](NC(=O)c1cc(-c2ccc(C#N)cc2)cc2ccn(Cc3cc(F)c4ccccc4n3)c12)c1ccc(C(=O)O)cc1 ; N#Cc1ccc(-c2cc(C(=O)NC3(c4ccc(C(=O)O)cc4)CC3)c3c(ccn3Cc3ccc4c(F)ccnc4c3)c2)cc1]

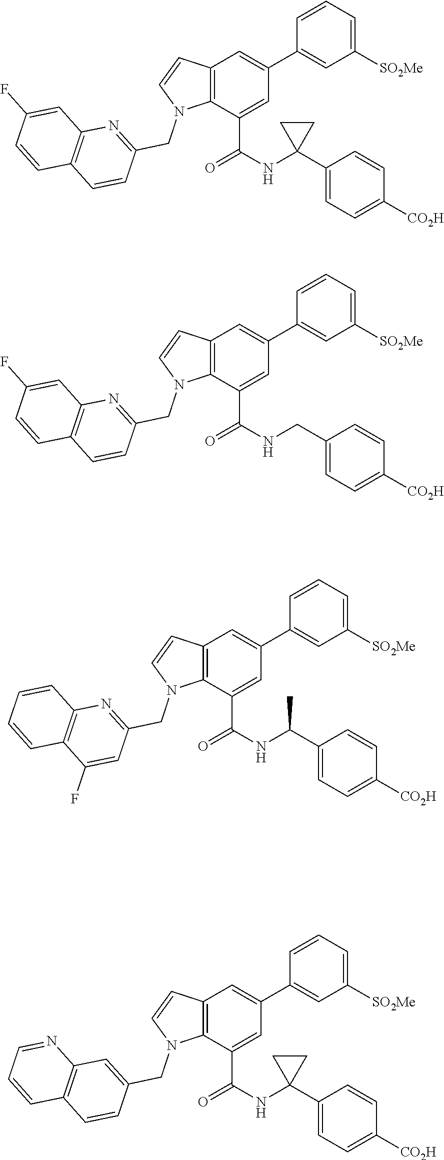 [SMILES: CS(=O)(=O)c1cccc(-c2cc(C(=O)NC3(c4ccc(C(=O)O)cc4)CC3)c3c(ccn3Cc3ccc4ccc(F)cc4n3)c2)c1 ; CS(=O)(=O)c1cccc(-c2cc(C(=O)NCc3ccc(C(=O)O)cc3)c3c(ccn3Cc3ccc4ccc(F)cc4n3)c2)c1 ; C[C@H](NC(=O)c1cc(-c2cccc(S(C)(=O)=O)c2)cc2ccn(Cc3cc(F)c4ccccc4n3)c12)c1ccc(C(=O)O)cc1 ; CS(=O)(=O)c1cccc(-c2cc(C(=O)NC3(c4ccc(C(=O)O)cc4)CC3)c3c(ccn3Cc3ccc4cccnc4c3)c2)c1]

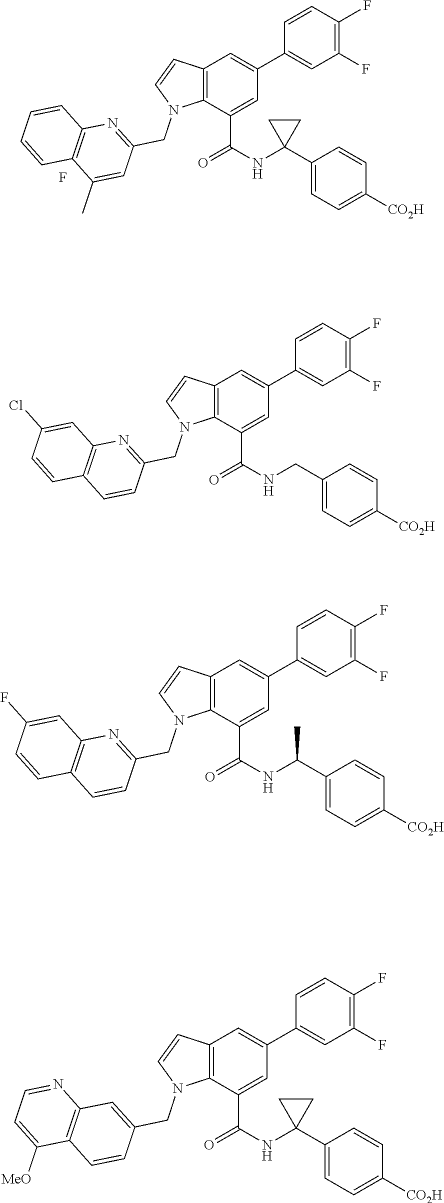

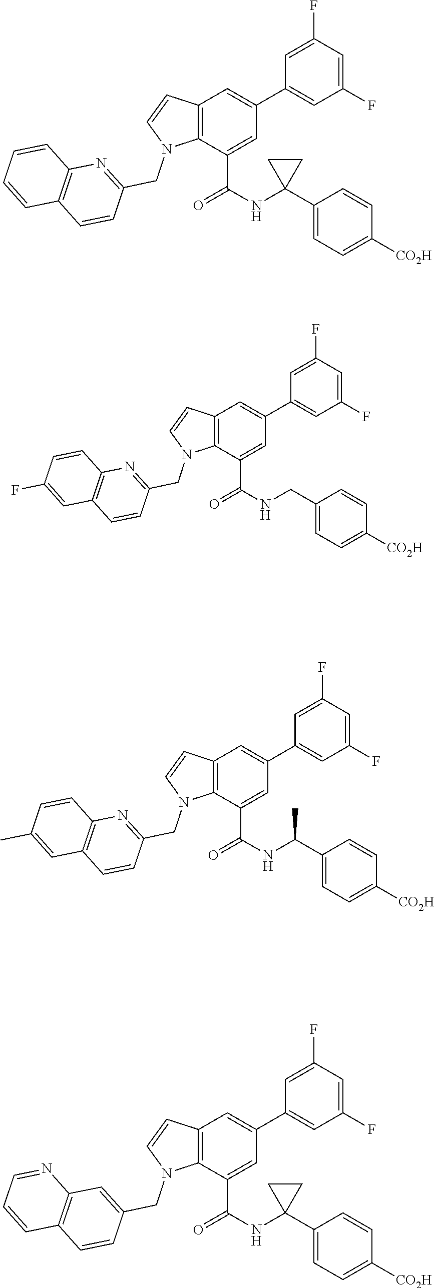 [SMILES: O=C(O)c1ccc(C2(NC(=O)c3cc(-c4cc(F)cc(F)c4)cc4ccn(Cc5ccc6ccccc6n5)c34)CC2)cc1 ; O=C(O)c1ccc(CNC(=O)c2cc(-c3cc(F)cc(F)c3)cc3ccn(Cc4ccc5cc(F)ccc5n4)c23)cc1 ; Cc1ccc2nc(Cn3ccc4cc(-c5cc(F)cc(F)c5)cc(C(=O)N[C@@H](C)c5ccc(C(=O)O)cc5)c43)ccc2c1 ; O=C(O)c1ccc(C2(NC(=O)c3cc(-c4cc(F)cc(F)c4)cc4ccn(Cc5ccc6cccnc6c5)c34)CC2)cc1]

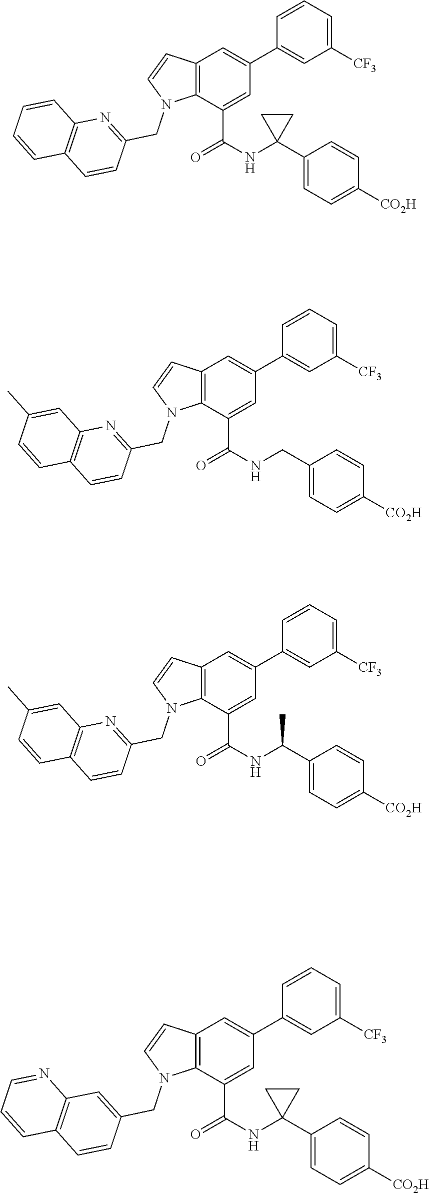 [SMILES: O=C(O)c1ccc(C2(NC(=O)c3cc(-c4cccc(C(F)(F)F)c4)cc4ccn(Cc5ccc6ccccc6n5)c34)CC2)cc1 ; Cc1ccc2ccc(Cn3ccc4cc(-c5cccc(C(F)(F)F)c5)cc(C(=O)NCc5ccc(C(=O)O)cc5)c43)nc2c1 ; Cc1ccc2ccc(Cn3ccc4cc(-c5cccc(C(F)(F)F)c5)cc(C(=O)N[C@@H](C)c5ccc(C(=O)O)cc5)c43)nc2c1 ; O=C(O)c1ccc(C2(NC(=O)c3cc(-c4cccc(C(F)(F)F)c4)cc4ccn(Cc5ccc6cccnc6c5)c34)CC2)cc1]

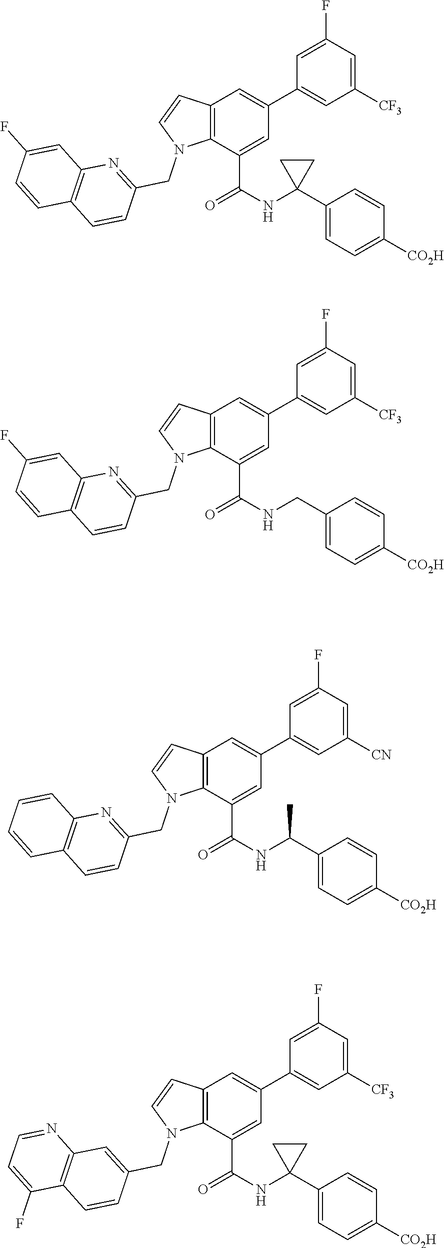 [SMILES: O=C(O)c1ccc(C2(NC(=O)c3cc(-c4cc(F)cc(C(F)(F)F)c4)cc4ccn(Cc5ccc6ccc(F)cc6n5)c34)CC2)cc1 ; O=C(O)c1ccc(CNC(=O)c2cc(-c3cc(F)cc(C(F)(F)F)c3)cc3ccn(Cc4ccc5ccc(F)cc5n4)c23)cc1 ; C[C@H](NC(=O)c1cc(-c2cc(F)cc(C#N)c2)cc2ccn(Cc3ccc4ccccc4n3)c12)c1ccc(C(=O)O)cc1 ; O=C(O)c1ccc(C2(NC(=O)c3cc(-c4cc(F)cc(C(F)(F)F)c4)cc4ccn(Cc5ccc6c(F)ccnc6c5)c34)CC2)cc1]

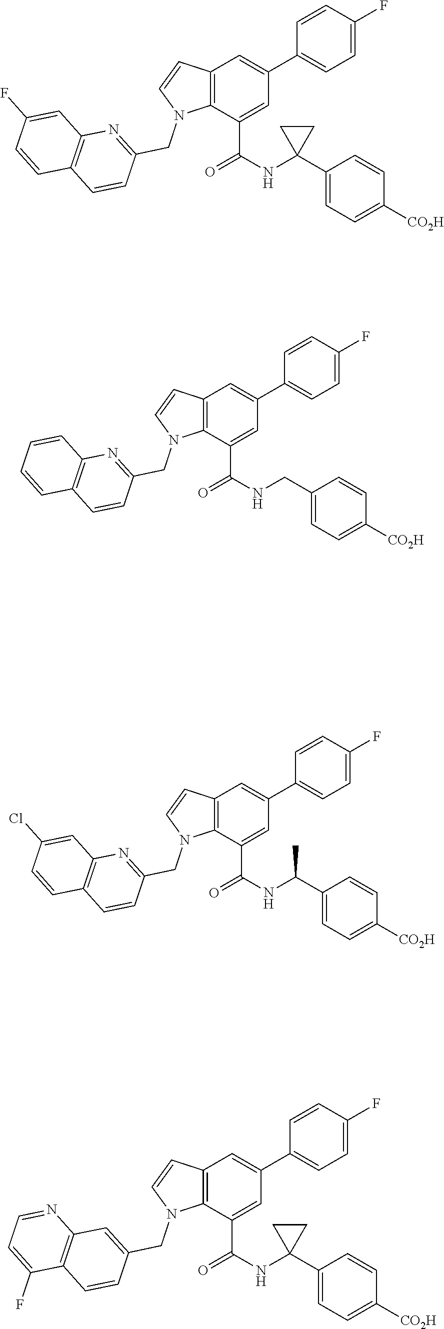 [SMILES: O=C(O)c1ccc(C2(NC(=O)c3cc(-c4ccc(F)cc4)cc4ccn(Cc5ccc6ccc(F)cc6n5)c34)CC2)cc1 ; O=C(O)c1ccc(CNC(=O)c2cc(-c3ccc(F)cc3)cc3ccn(Cc4ccc5ccccc5n4)c23)cc1 ; C[C@H](NC(=O)c1cc(-c2ccc(F)cc2)cc2ccn(Cc3ccc4ccc(Cl)cc4n3)c12)c1ccc(C(=O)O)cc1 ; O=C(O)c1ccc(C2(NC(=O)c3cc(-c4ccc(F)cc4)cc4ccn(Cc5ccc6c(F)ccnc6c5)c34)CC2)cc1]

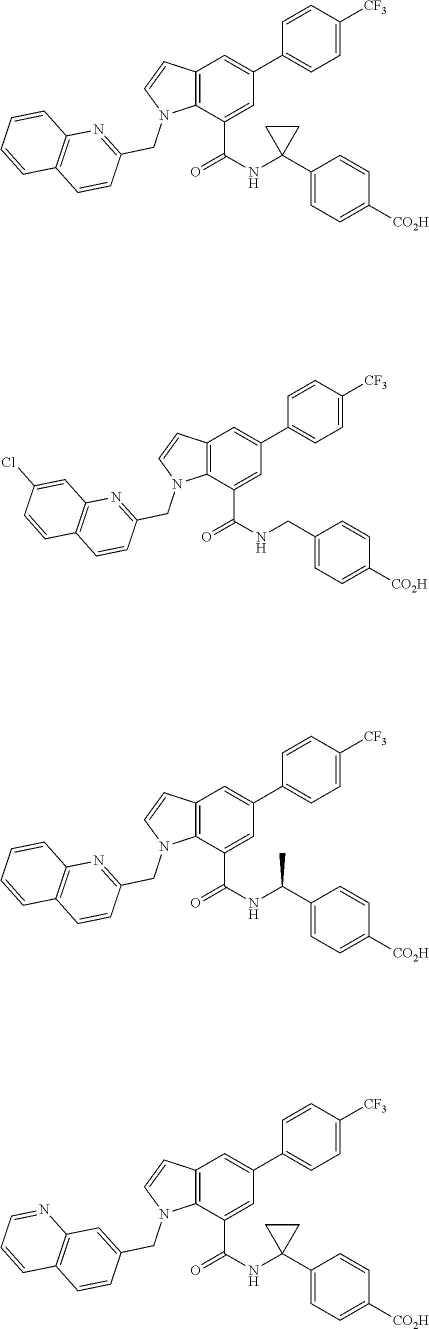 [SMILES: O=C(O)c1ccc(C2(NC(=O)c3cc(-c4ccc(C(F)(F)F)cc4)cc4ccn(Cc5ccc6ccccc6n5)c34)CC2)cc1 ; O=C(O)c1ccc(CNC(=O)c2cc(-c3ccc(C(F)(F)F)cc3)cc3ccn(Cc4ccc5ccc(Cl)cc5n4)c23)cc1 ; C[C@H](NC(=O)c1cc(-c2ccc(C(F)(F)F)cc2)cc2ccn(Cc3ccc4ccccc4n3)c12)c1ccc(C(=O)O)cc1 ; O=C(O)c1ccc(C2(NC(=O)c3cc(-c4ccc(C(F)(F)F)cc4)cc4ccn(Cc5ccc6cccnc6c5)c34)CC2)cc1]

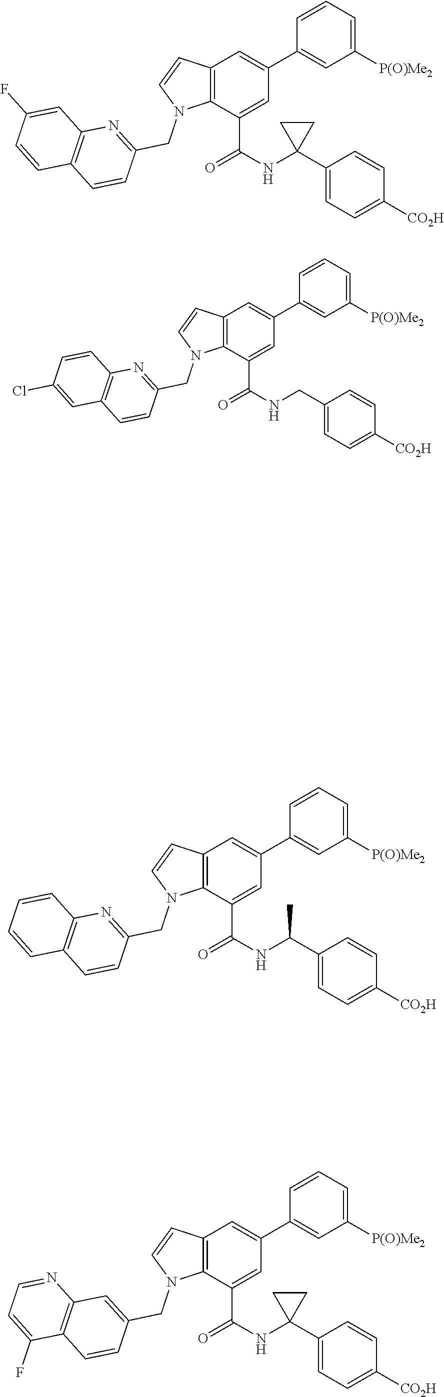

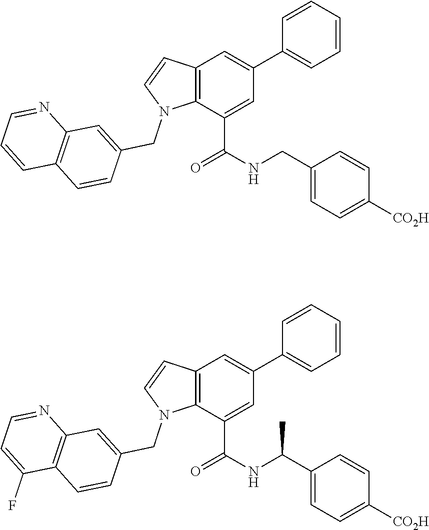 [SMILES: O=C(O)c1ccc(CNC(=O)c2cc(-c3ccccc3)cc3ccn(Cc4ccc5cccnc5c4)c23)cc1 ; C[C@H](NC(=O)c1cc(-c2ccccc2)cc2ccn(Cc3ccc4c(F)ccnc4c3)c12)c1ccc(C(=O)O)cc1]

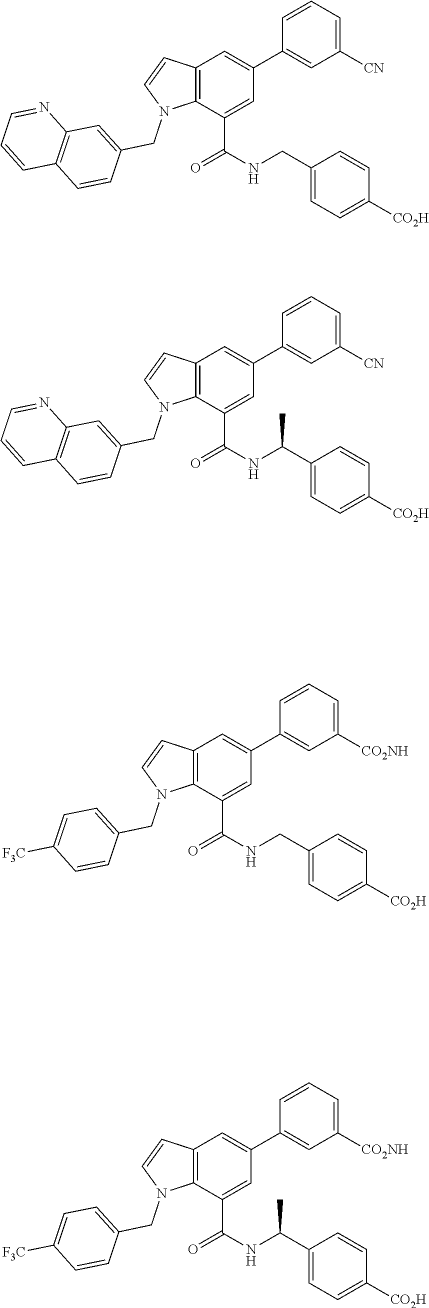

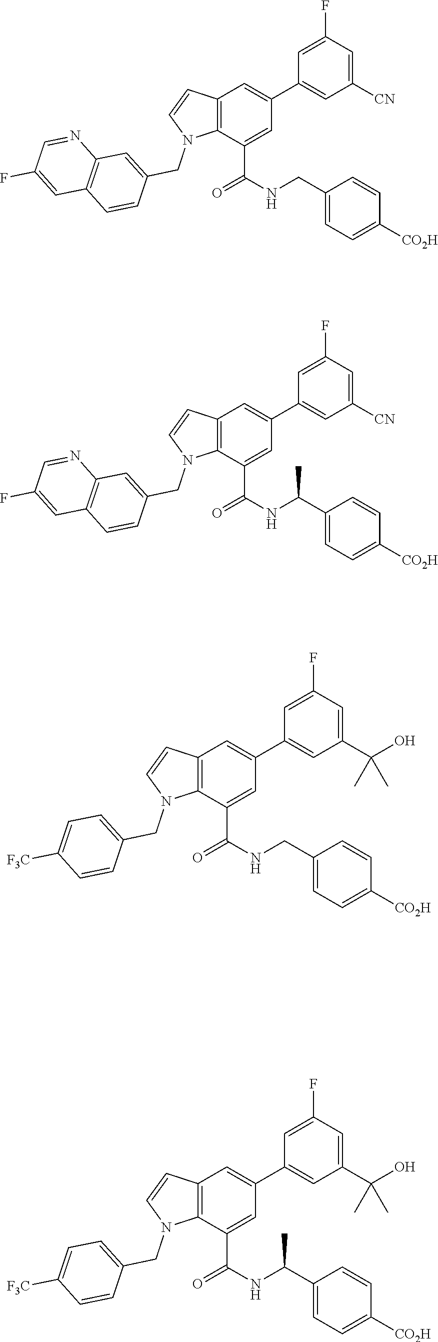 [SMILES: N#Cc1cc(F)cc(-c2cc(C(=O)NCc3ccc(C(=O)O)cc3)c3c(ccn3Cc3ccc4cc(F)cnc4c3)c2)c1 ; C[C@H](NC(=O)c1cc(-c2cc(F)cc(C#N)c2)cc2ccn(Cc3ccc4cc(F)cnc4c3)c12)c1ccc(C(=O)O)cc1 ; CC(C)(O)c1cc(F)cc(-c2cc(C(=O)NCc3ccc(C(=O)O)cc3)c3c(ccn3Cc3ccc(C(F)(F)F)cc3)c2)c1 ; C[C@H](NC(=O)c1cc(-c2cc(F)cc(C(C)(C)O)c2)cc2ccn(Cc3ccc(C(F)(F)F)cc3)c12)c1ccc(C(=O)O)cc1]

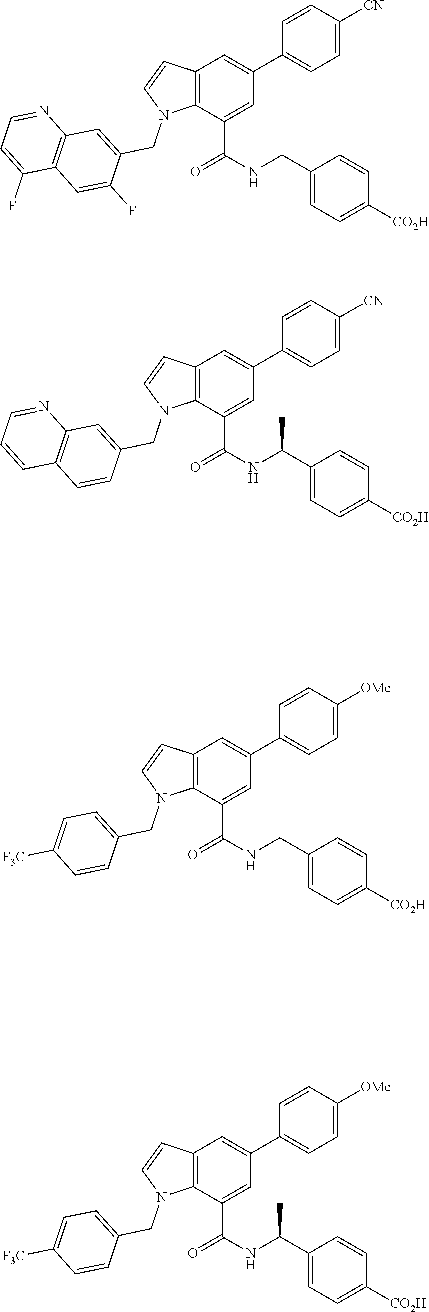 [SMILES: N#Cc1ccc(-c2cc(C(=O)NCc3ccc(C(=O)O)cc3)c3c(ccn3Cc3cc4nccc(F)c4cc3F)c2)cc1 ; C[C@H](NC(=O)c1cc(-c2ccc(C#N)cc2)cc2ccn(Cc3ccc4cccnc4c3)c12)c1ccc(C(=O)O)cc1 ; COc1ccc(-c2cc(C(=O)NCc3ccc(C(=O)O)cc3)c3c(ccn3Cc3ccc(C(F)(F)F)cc3)c2)cc1 ; COc1ccc(-c2cc(C(=O)N[C@@H](C)c3ccc(C(=O)O)cc3)c3c(ccn3Cc3ccc(C(F)(F)F)cc3)c2)cc1]

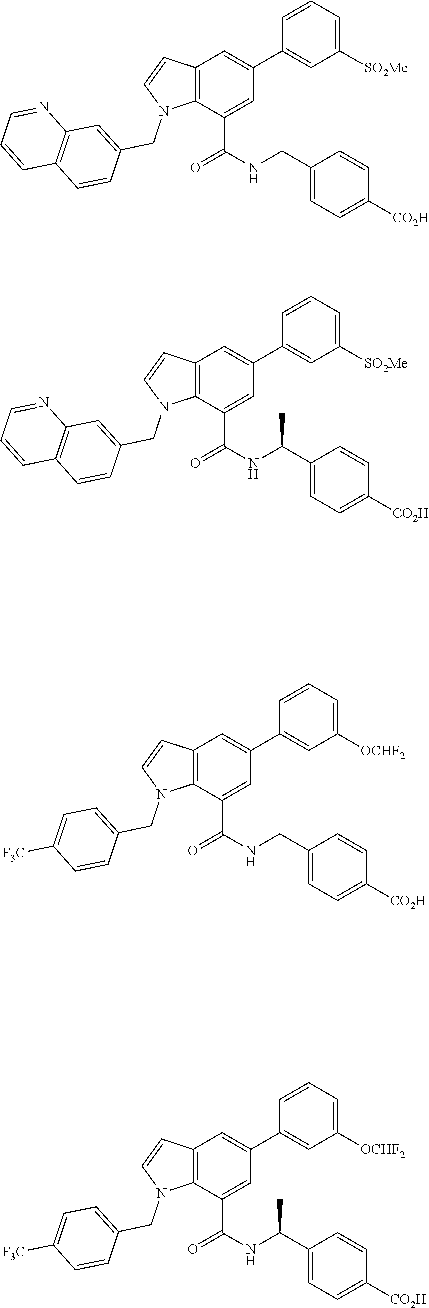 [SMILES: CS(=O)(=O)c1cccc(-c2cc(C(=O)NCc3ccc(C(=O)O)cc3)c3c(ccn3Cc3ccc4cccnc4c3)c2)c1 ; C[C@H](NC(=O)c1cc(-c2cccc(S(C)(=O)=O)c2)cc2ccn(Cc3ccc4cccnc4c3)c12)c1ccc(C(=O)O)cc1 ; O=C(O)c1ccc(CNC(=O)c2cc(-c3cccc(OC(F)F)c3)cc3ccn(Cc4ccc(C(F)(F)F)cc4)c23)cc1 ; C[C@H](NC(=O)c1cc(-c2cccc(OC(F)F)c2)cc2ccn(Cc3ccc(C(F)(F)F)cc3)c12)c1ccc(C(=O)O)cc1]

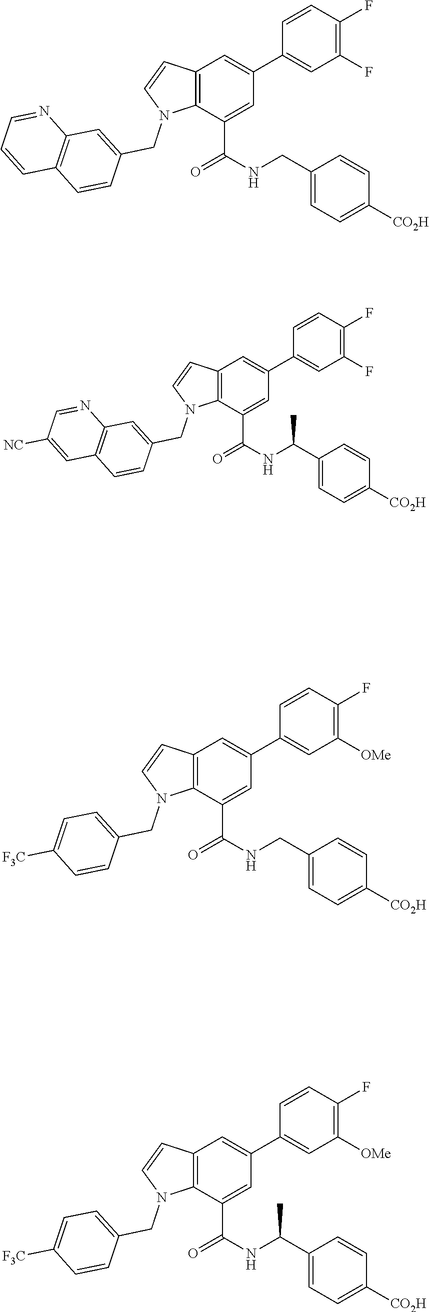 [SMILES: O=C(O)c1ccc(CNC(=O)c2cc(-c3ccc(F)c(F)c3)cc3ccn(Cc4ccc5cccnc5c4)c23)cc1 ; C[C@H](NC(=O)c1cc(-c2ccc(F)c(F)c2)cc2ccn(Cc3ccc4cc(C#N)cnc4c3)c12)c1ccc(C(=O)O)cc1 ; COc1cc(-c2cc(C(=O)NCc3ccc(C(=O)O)cc3)c3c(ccn3Cc3ccc(C(F)(F)F)cc3)c2)ccc1F ; COc1cc(-c2cc(C(=O)N[C@@H](C)c3ccc(C(=O)O)cc3)c3c(ccn3Cc3ccc(C(F)(F)F)cc3)c2)ccc1F]

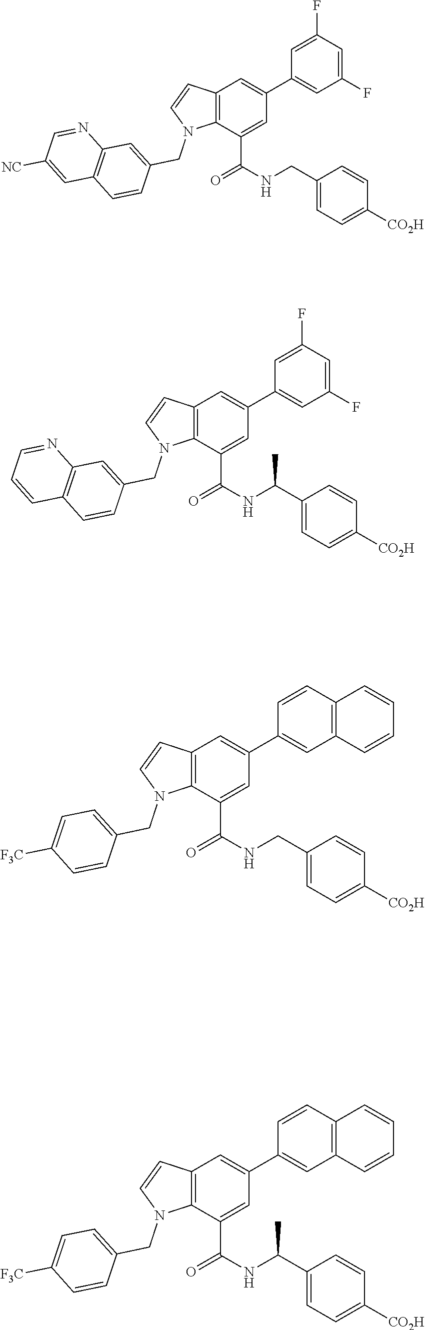 [SMILES: N#Cc1cnc2cc(Cn3ccc4cc(-c5cc(F)cc(F)c5)cc(C(=O)NCc5ccc(C(=O)O)cc5)c43)ccc2c1 ; C[C@H](NC(=O)c1cc(-c2cc(F)cc(F)c2)cc2ccn(Cc3ccc4cccnc4c3)c12)c1ccc(C(=O)O)cc1 ; O=C(O)c1ccc(CNC(=O)c2cc(-c3ccc4ccccc4c3)cc3ccn(Cc4ccc(C(F)(F)F)cc4)c23)cc1 ; C[C@H](NC(=O)c1cc(-c2ccc3ccccc3c2)cc2ccn(Cc3ccc(C(F)(F)F)cc3)c12)c1ccc(C(=O)O)cc1]

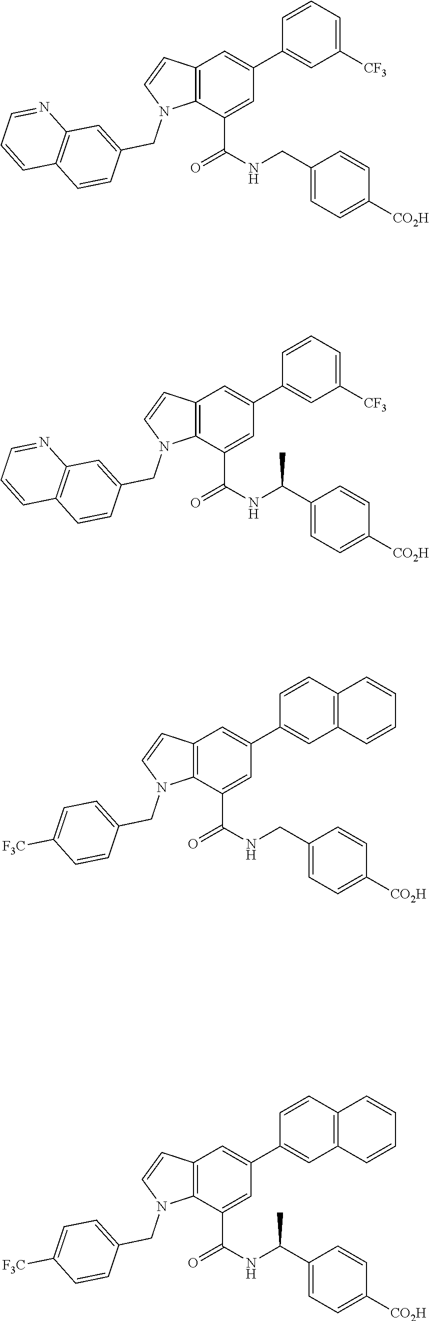 [SMILES: O=C(O)c1ccc(CNC(=O)c2cc(-c3cccc(C(F)(F)F)c3)cc3ccn(Cc4ccc5cccnc5c4)c23)cc1 ; C[C@H](NC(=O)c1cc(-c2cccc(C(F)(F)F)c2)cc2ccn(Cc3ccc4cccnc4c3)c12)c1ccc(C(=O)O)cc1 ; O=C(O)c1ccc(CNC(=O)c2cc(-c3ccc4ccccc4c3)cc3ccn(Cc4ccc(C(F)(F)F)cc4)c23)cc1 ; C[C@H](NC(=O)c1cc(-c2ccc3ccccc3c2)cc2ccn(Cc3ccc(C(F)(F)F)cc3)c12)c1ccc(C(=O)O)cc1]

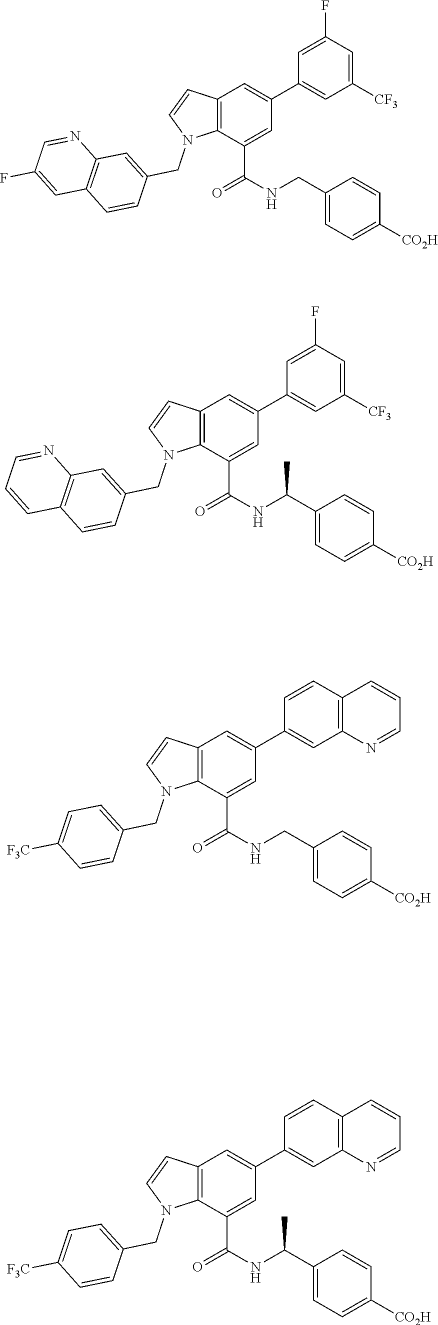 [SMILES: O=C(O)c1ccc(CNC(=O)c2cc(-c3cc(F)cc(C(F)(F)F)c3)cc3ccn(Cc4ccc5cc(F)cnc5c4)c23)cc1 ; C[C@H](NC(=O)c1cc(-c2cc(F)cc(C(F)(F)F)c2)cc2ccn(Cc3ccc4cccnc4c3)c12)c1ccc(C(=O)O)cc1 ; O=C(O)c1ccc(CNC(=O)c2cc(-c3ccc4cccnc4c3)cc3ccn(Cc4ccc(C(F)(F)F)cc4)c23)cc1 ; C[C@H](NC(=O)c1cc(-c2ccc3cccnc3c2)cc2ccn(Cc3ccc(C(F)(F)F)cc3)c12)c1ccc(C(=O)O)cc1]

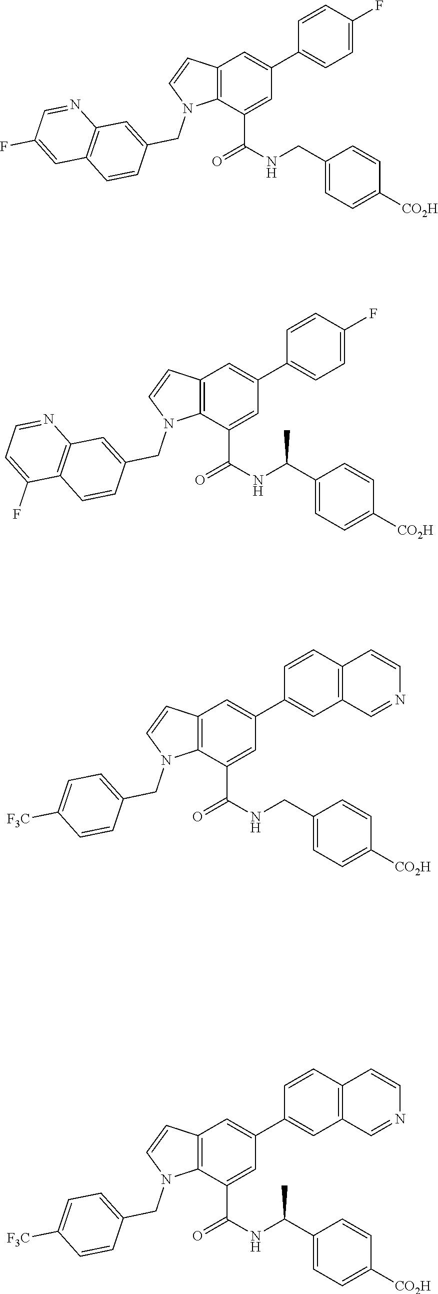 [SMILES: O=C(O)c1ccc(CNC(=O)c2cc(-c3ccc(F)cc3)cc3ccn(Cc4ccc5cc(F)cnc5c4)c23)cc1 ; C[C@H](NC(=O)c1cc(-c2ccc(F)cc2)cc2ccn(Cc3ccc4c(F)ccnc4c3)c12)c1ccc(C(=O)O)cc1 ; O=C(O)c1ccc(CNC(=O)c2cc(-c3ccc4ccncc4c3)cc3ccn(Cc4ccc(C(F)(F)F)cc4)c23)cc1 ; C[C@H](NC(=O)c1cc(-c2ccc3ccncc3c2)cc2ccn(Cc3ccc(C(F)(F)F)cc3)c12)c1ccc(C(=O)O)cc1]

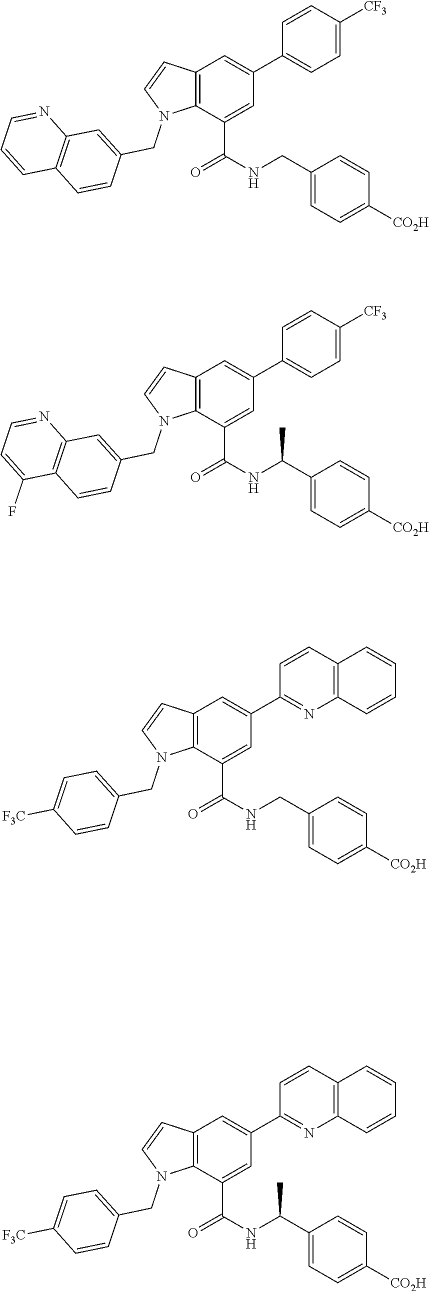 [SMILES: O=C(O)c1ccc(CNC(=O)c2cc(-c3ccc(C(F)(F)F)cc3)cc3ccn(Cc4ccc5cccnc5c4)c23)cc1 ; C[C@H](NC(=O)c1cc(-c2ccc(C(F)(F)F)cc2)cc2ccn(Cc3ccc4c(F)ccnc4c3)c12)c1ccc(C(=O)O)cc1 ; O=C(O)c1ccc(CNC(=O)c2cc(-c3ccc4ccccc4n3)cc3ccn(Cc4ccc(C(F)(F)F)cc4)c23)cc1 ; C[C@H](NC(=O)c1cc(-c2ccc3ccccc3n2)cc2ccn(Cc3ccc(C(F)(F)F)cc3)c12)c1ccc(C(=O)O)cc1]

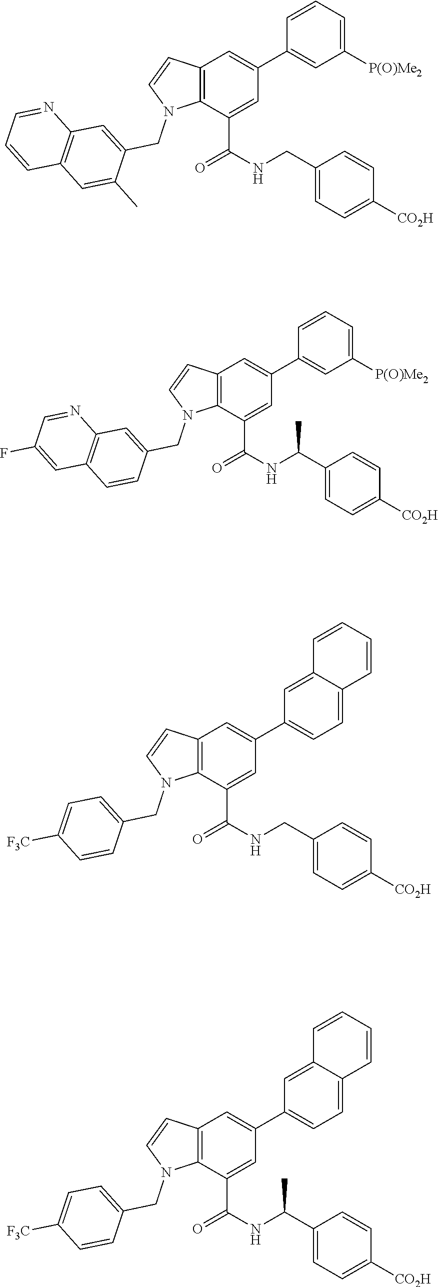

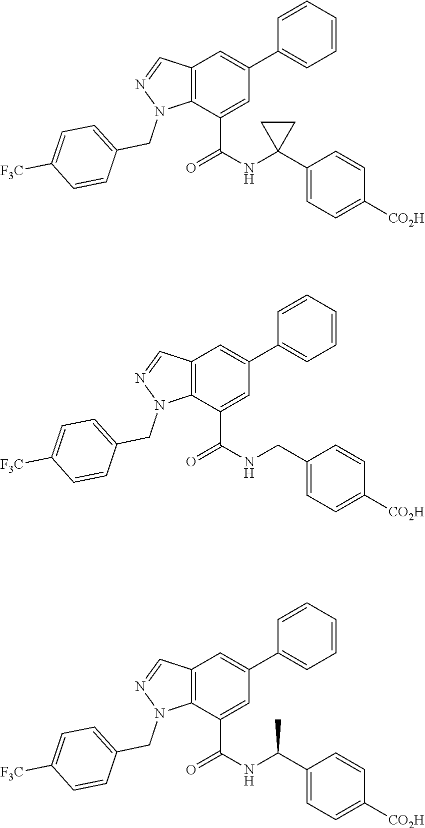 [SMILES: O=C(O)c1ccc(C2(NC(=O)c3cc(-c4ccccc4)cc4cnn(Cc5ccc(C(F)(F)F)cc5)c34)CC2)cc1 ; O=C(O)c1ccc(CNC(=O)c2cc(-c3ccccc3)cc3cnn(Cc4ccc(C(F)(F)F)cc4)c23)cc1 ; C[C@H](NC(=O)c1cc(-c2ccccc2)cc2cnn(Cc3ccc(C(F)(F)F)cc3)c12)c1ccc(C(=O)O)cc1]

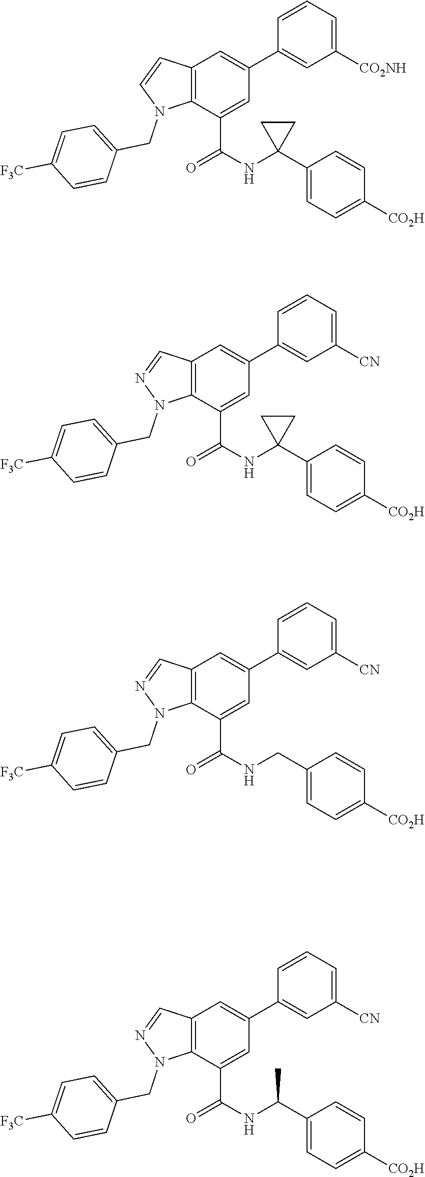

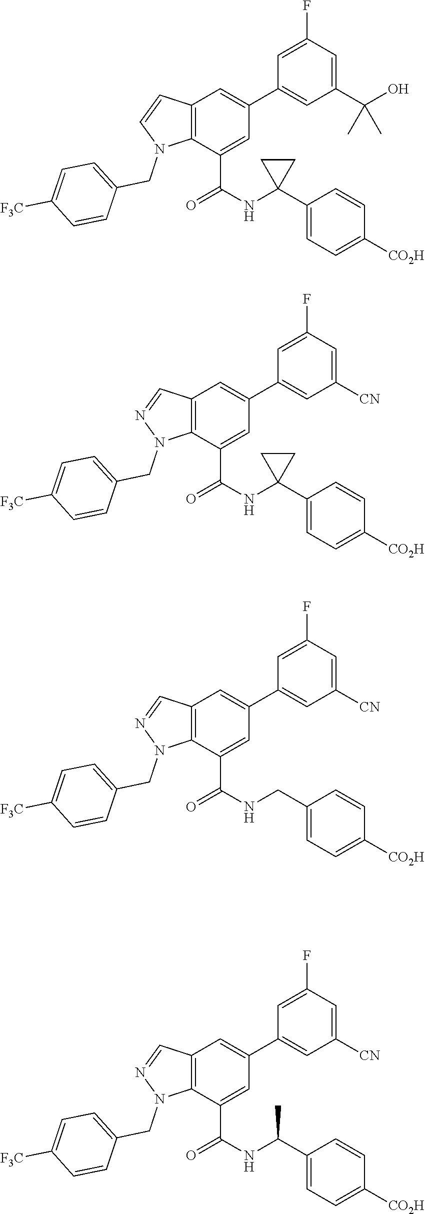 [SMILES: CC(C)(O)c1cc(F)cc(-c2cc(C(=O)NC3(c4ccc(C(=O)O)cc4)CC3)c3c(ccn3Cc3ccc(C(F)(F)F)cc3)c2)c1 ; N#Cc1cc(F)cc(-c2cc(C(=O)NC3(c4ccc(C(=O)O)cc4)CC3)c3c(cnn3Cc3ccc(C(F)(F)F)cc3)c2)c1 ; N#Cc1cc(F)cc(-c2cc(C(=O)NCc3ccc(C(=O)O)cc3)c3c(cnn3Cc3ccc(C(F)(F)F)cc3)c2)c1 ; C[C@H](NC(=O)c1cc(-c2cc(F)cc(C#N)c2)cc2cnn(Cc3ccc(C(F)(F)F)cc3)c12)c1ccc(C(=O)O)cc1]

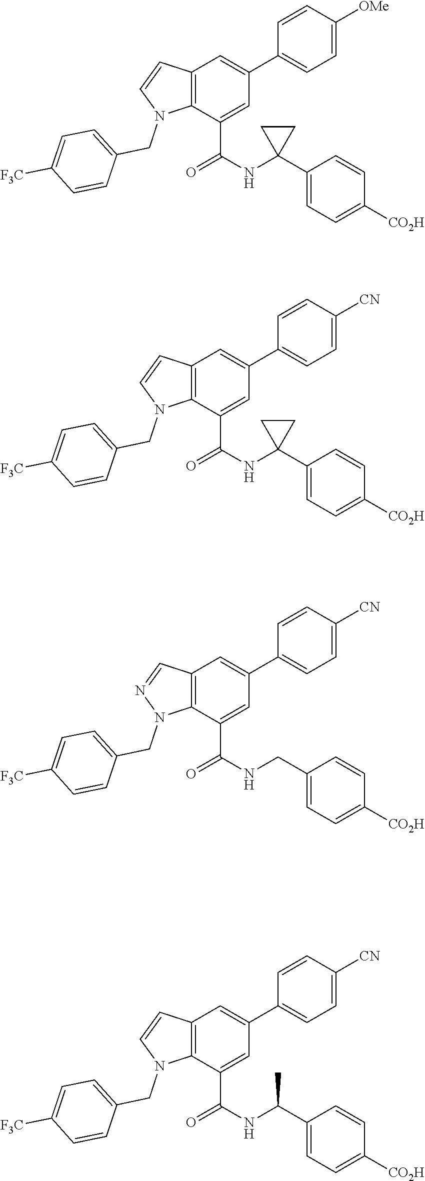 [SMILES: COc1ccc(-c2cc(C(=O)NC3(c4ccc(C(=O)O)cc4)CC3)c3c(ccn3Cc3ccc(C(F)(F)F)cc3)c2)cc1 ; N#Cc1ccc(-c2cc(C(=O)NC3(c4ccc(C(=O)O)cc4)CC3)c3c(ccn3Cc3ccc(C(F)(F)F)cc3)c2)cc1 ; N#Cc1ccc(-c2cc(C(=O)NCc3ccc(C(=O)O)cc3)c3c(cnn3Cc3ccc(C(F)(F)F)cc3)c2)cc1 ; C[C@H](NC(=O)c1cc(-c2ccc(C#N)cc2)cc2ccn(Cc3ccc(C(F)(F)F)cc3)c12)c1ccc(C(=O)O)cc1]

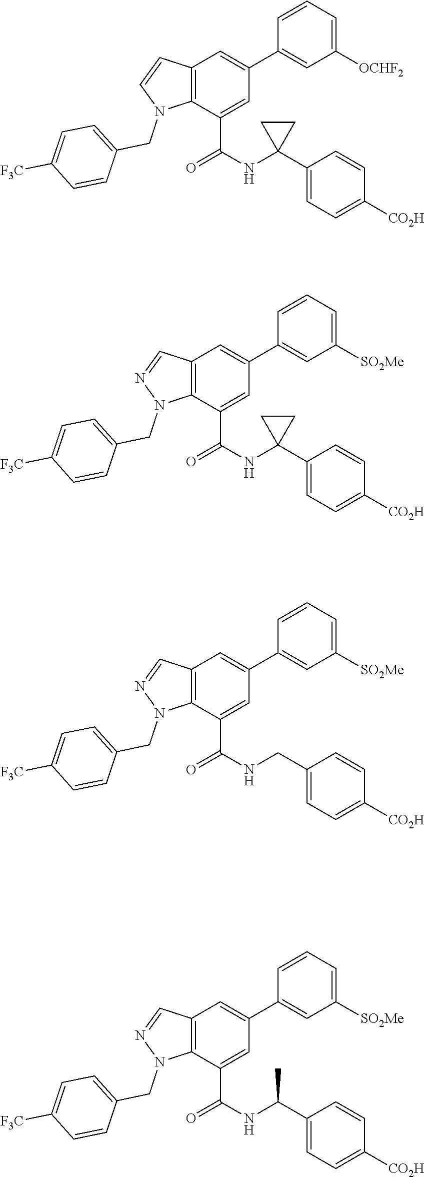 [SMILES: O=C(O)c1ccc(C2(NC(=O)c3cc(-c4cccc(OC(F)F)c4)cc4ccn(Cc5ccc(C(F)(F)F)cc5)c34)CC2)cc1 ; CS(=O)(=O)c1cccc(-c2cc(C(=O)NC3(c4ccc(C(=O)O)cc4)CC3)c3c(cnn3Cc3ccc(C(F)(F)F)cc3)c2)c1 ; CS(=O)(=O)c1cccc(-c2cc(C(=O)NCc3ccc(C(=O)O)cc3)c3c(cnn3Cc3ccc(C(F)(F)F)cc3)c2)c1 ; C[C@H](NC(=O)c1cc(-c2cccc(S(C)(=O)=O)c2)cc2cnn(Cc3ccc(C(F)(F)F)cc3)c12)c1ccc(C(=O)O)cc1]

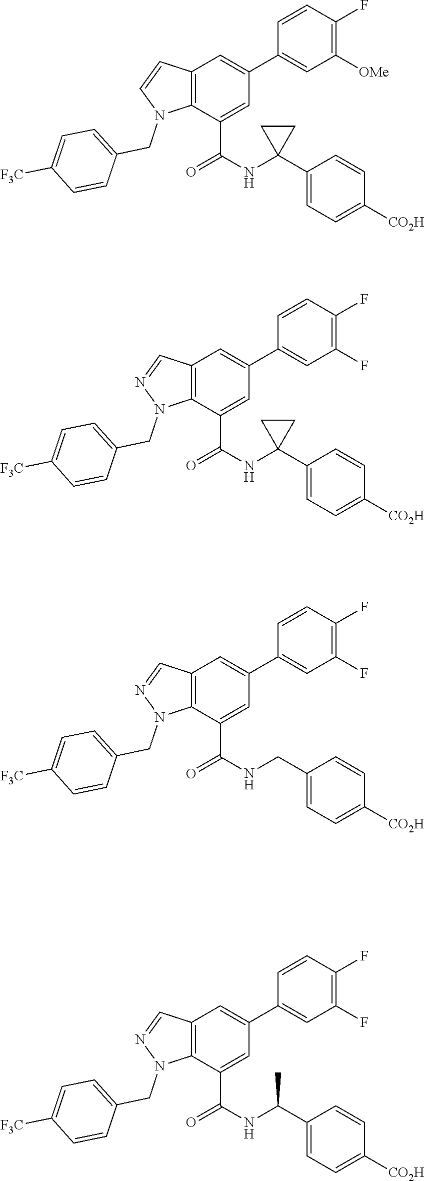 [SMILES: COc1cc(-c2cc(C(=O)NC3(c4ccc(C(=O)O)cc4)CC3)c3c(ccn3Cc3ccc(C(F)(F)F)cc3)c2)ccc1F ; O=C(O)c1ccc(C2(NC(=O)c3cc(-c4ccc(F)c(F)c4)cc4cnn(Cc5ccc(C(F)(F)F)cc5)c34)CC2)cc1 ; O=C(O)c1ccc(CNC(=O)c2cc(-c3ccc(F)c(F)c3)cc3cnn(Cc4ccc(C(F)(F)F)cc4)c23)cc1 ; C[C@H](NC(=O)c1cc(-c2ccc(F)c(F)c2)cc2cnn(Cc3ccc(C(F)(F)F)cc3)c12)c1ccc(C(=O)O)cc1]

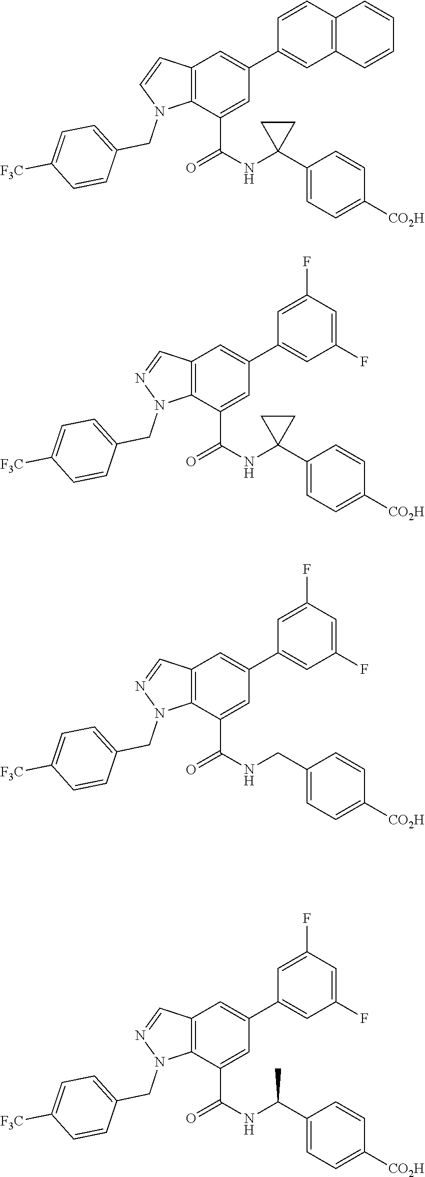 [SMILES: O=C(O)c1ccc(C2(NC(=O)c3cc(-c4ccc5ccccc5c4)cc4ccn(Cc5ccc(C(F)(F)F)cc5)c34)CC2)cc1 ; O=C(O)c1ccc(C2(NC(=O)c3cc(-c4cc(F)cc(F)c4)cc4cnn(Cc5ccc(C(F)(F)F)cc5)c34)CC2)cc1 ; O=C(O)c1ccc(CNC(=O)c2cc(-c3cc(F)cc(F)c3)cc3cnn(Cc4ccc(C(F)(F)F)cc4)c23)cc1 ; C[C@H](NC(=O)c1cc(-c2cc(F)cc(F)c2)cc2cnn(Cc3ccc(C(F)(F)F)cc3)c12)c1ccc(C(=O)O)cc1]

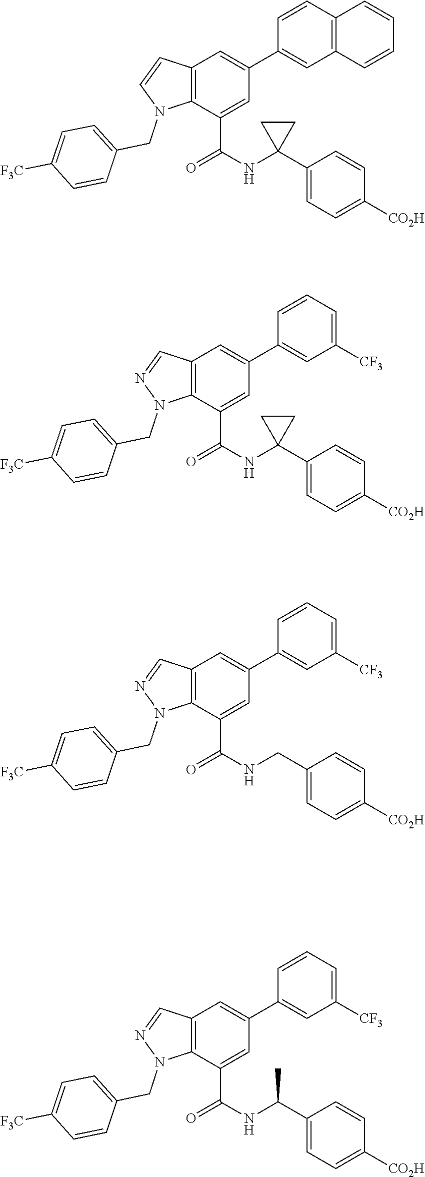 [SMILES: O=C(O)c1ccc(C2(NC(=O)c3cc(-c4ccc5ccccc5c4)cc4ccn(Cc5ccc(C(F)(F)F)cc5)c34)CC2)cc1 ; O=C(O)c1ccc(C2(NC(=O)c3cc(-c4cccc(C(F)(F)F)c4)cc4cnn(Cc5ccc(C(F)(F)F)cc5)c34)CC2)cc1 ; O=C(O)c1ccc(CNC(=O)c2cc(-c3cccc(C(F)(F)F)c3)cc3cnn(Cc4ccc(C(F)(F)F)cc4)c23)cc1 ; C[C@H](NC(=O)c1cc(-c2cccc(C(F)(F)F)c2)cc2cnn(Cc3ccc(C(F)(F)F)cc3)c12)c1ccc(C(=O)O)cc1]

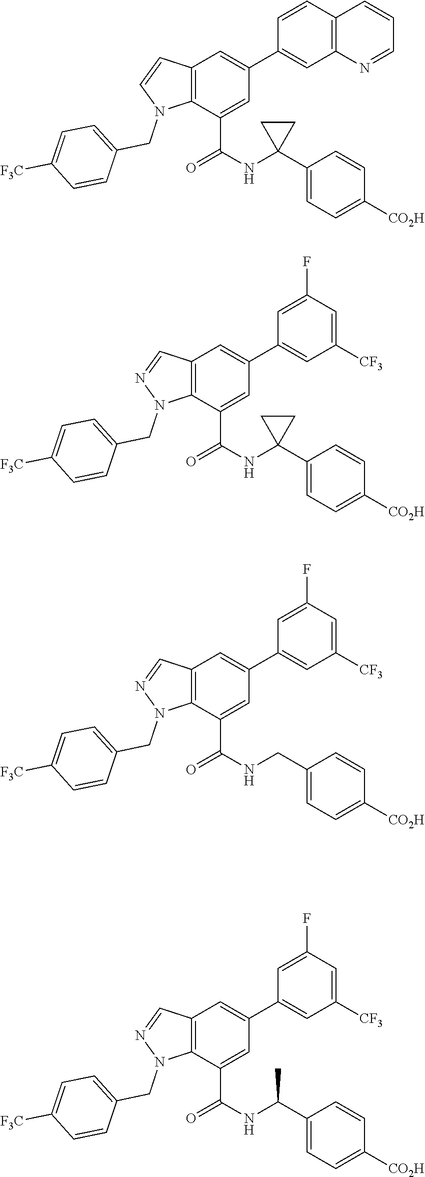 [SMILES: O=C(O)c1ccc(C2(NC(=O)c3cc(-c4ccc5cccnc5c4)cc4ccn(Cc5ccc(C(F)(F)F)cc5)c34)CC2)cc1 ; O=C(O)c1ccc(C2(NC(=O)c3cc(-c4cc(F)cc(C(F)(F)F)c4)cc4cnn(Cc5ccc(C(F)(F)F)cc5)c34)CC2)cc1 ; O=C(O)c1ccc(CNC(=O)c2cc(-c3cc(F)cc(C(F)(F)F)c3)cc3cnn(Cc4ccc(C(F)(F)F)cc4)c23)cc1 ; C[C@H](NC(=O)c1cc(-c2cc(F)cc(C(F)(F)F)c2)cc2cnn(Cc3ccc(C(F)(F)F)cc3)c12)c1ccc(C(=O)O)cc1]

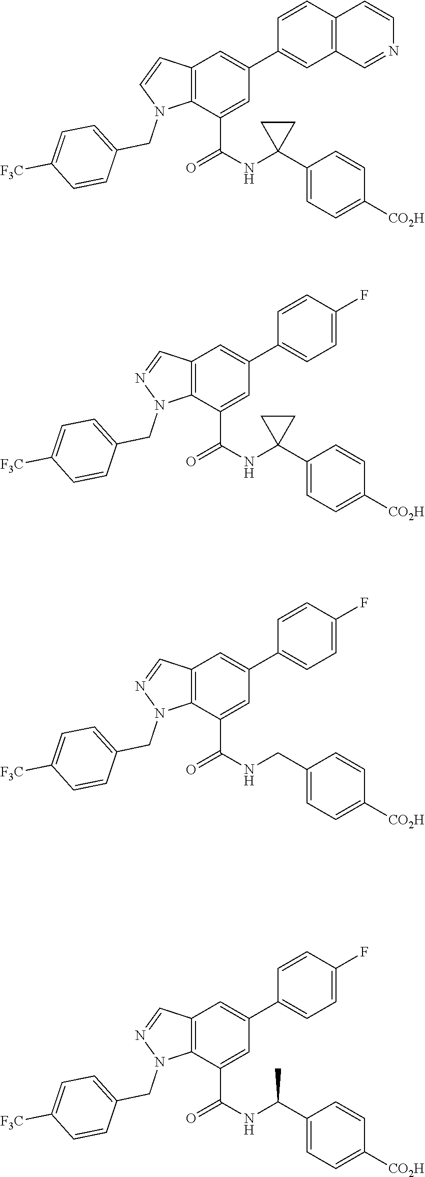 [SMILES: O=C(O)c1ccc(C2(NC(=O)c3cc(-c4ccc5ccncc5c4)cc4ccn(Cc5ccc(C(F)(F)F)cc5)c34)CC2)cc1 ; O=C(O)c1ccc(C2(NC(=O)c3cc(-c4ccc(F)cc4)cc4cnn(Cc5ccc(C(F)(F)F)cc5)c34)CC2)cc1 ; O=C(O)c1ccc(CNC(=O)c2cc(-c3ccc(F)cc3)cc3cnn(Cc4ccc(C(F)(F)F)cc4)c23)cc1 ; C[C@H](NC(=O)c1cc(-c2ccc(F)cc2)cc2cnn(Cc3ccc(C(F)(F)F)cc3)c12)c1ccc(C(=O)O)cc1]

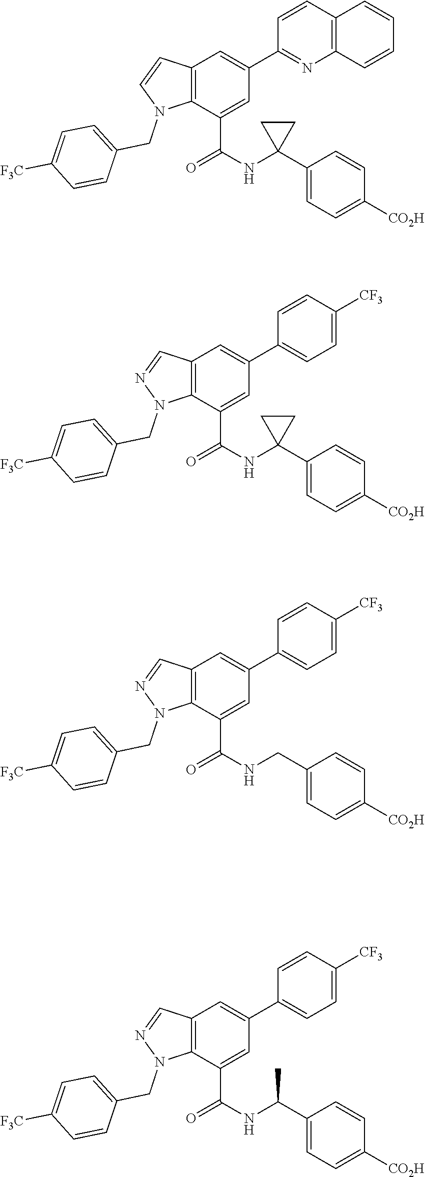 [SMILES: O=C(O)c1ccc(C2(NC(=O)c3cc(-c4ccc5ccccc5n4)cc4ccn(Cc5ccc(C(F)(F)F)cc5)c34)CC2)cc1 ; O=C(O)c1ccc(C2(NC(=O)c3cc(-c4ccc(C(F)(F)F)cc4)cc4cnn(Cc5ccc(C(F)(F)F)cc5)c34)CC2)cc1 ; O=C(O)c1ccc(CNC(=O)c2cc(-c3ccc(C(F)(F)F)cc3)cc3cnn(Cc4ccc(C(F)(F)F)cc4)c23)cc1 ; C[C@H](NC(=O)c1cc(-c2ccc(C(F)(F)F)cc2)cc2cnn(Cc3ccc(C(F)(F)F)cc3)c12)c1ccc(C(=O)O)cc1]

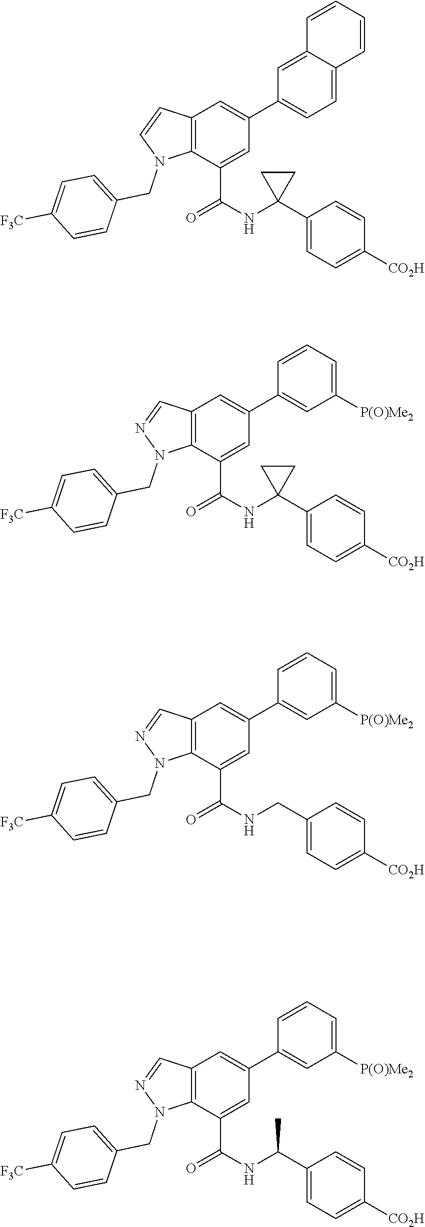

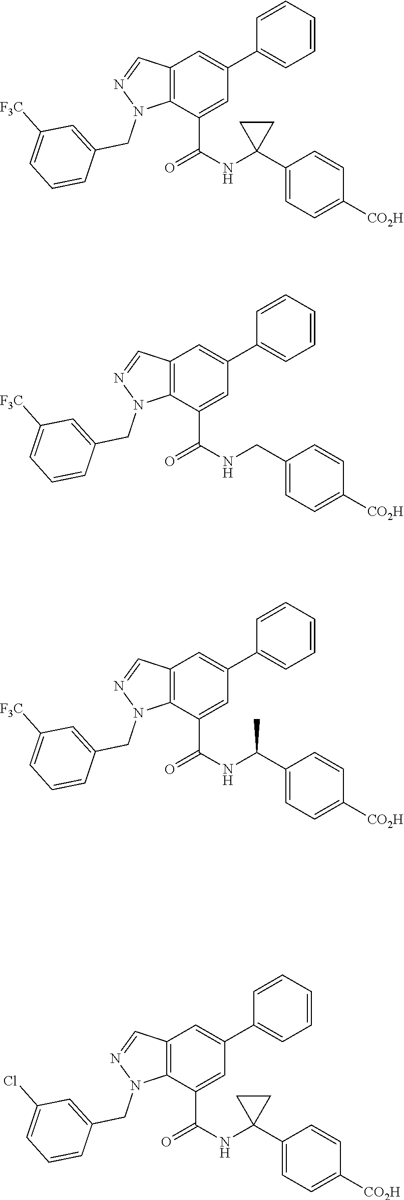 [SMILES: O=C(O)c1ccc(C2(NC(=O)c3cc(-c4ccccc4)cc4cnn(Cc5cccc(C(F)(F)F)c5)c34)CC2)cc1 ; O=C(O)c1ccc(CNC(=O)c2cc(-c3ccccc3)cc3cnn(Cc4cccc(C(F)(F)F)c4)c23)cc1 ; C[C@H](NC(=O)c1cc(-c2ccccc2)cc2cnn(Cc3cccc(C(F)(F)F)c3)c12)c1ccc(C(=O)O)cc1 ; O=C(O)c1ccc(C2(NC(=O)c3cc(-c4ccccc4)cc4cnn(Cc5cccc(Cl)c5)c34)CC2)cc1]

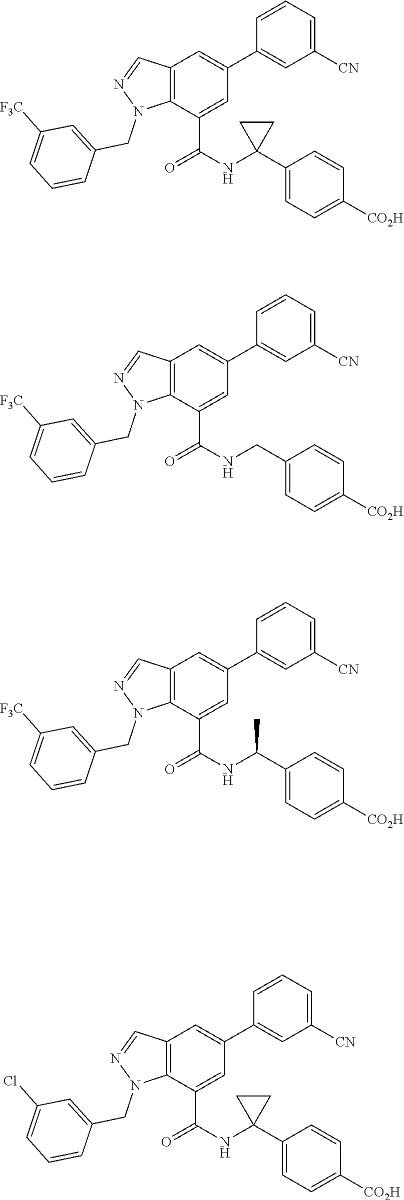 [SMILES: N#Cc1cccc(-c2cc(C(=O)NC3(c4ccc(C(=O)O)cc4)CC3)c3c(cnn3Cc3cccc(C(F)(F)F)c3)c2)c1 ; N#Cc1cccc(-c2cc(C(=O)NCc3ccc(C(=O)O)cc3)c3c(cnn3Cc3cccc(C(F)(F)F)c3)c2)c1 ; C[C@H](NC(=O)c1cc(-c2cccc(C#N)c2)cc2cnn(Cc3cccc(C(F)(F)F)c3)c12)c1ccc(C(=O)O)cc1 ; N#Cc1cccc(-c2cc(C(=O)NC3(c4ccc(C(=O)O)cc4)CC3)c3c(cnn3Cc3cccc(Cl)c3)c2)c1]

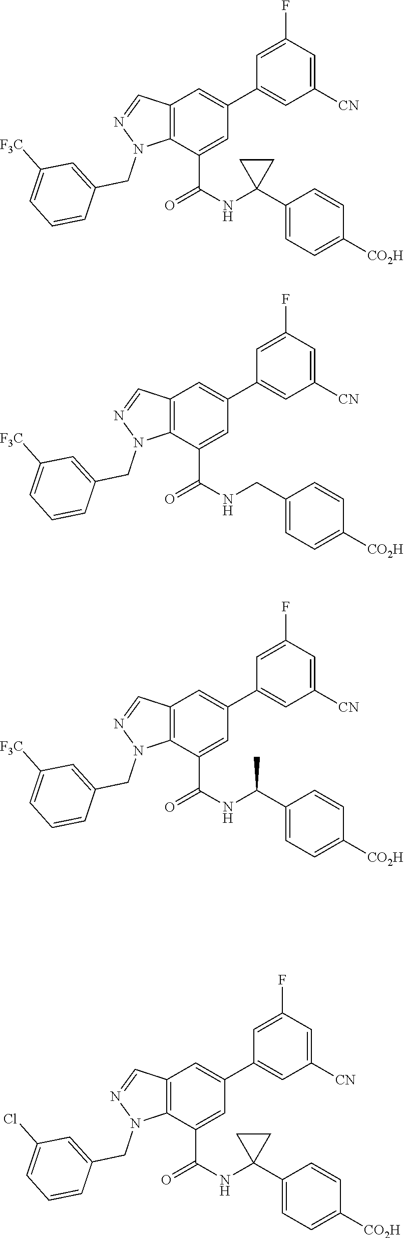 [SMILES: N#Cc1cc(F)cc(-c2cc(C(=O)NC3(c4ccc(C(=O)O)cc4)CC3)c3c(cnn3Cc3cccc(C(F)(F)F)c3)c2)c1 ; N#Cc1cc(F)cc(-c2cc(C(=O)NCc3ccc(C(=O)O)cc3)c3c(cnn3Cc3cccc(C(F)(F)F)c3)c2)c1 ; C[C@H](NC(=O)c1cc(-c2cc(F)cc(C#N)c2)cc2cnn(Cc3cccc(C(F)(F)F)c3)c12)c1ccc(C(=O)O)cc1 ; N#Cc1cc(F)cc(-c2cc(C(=O)NC3(c4ccc(C(=O)O)cc4)CC3)c3c(cnn3Cc3cccc(Cl)c3)c2)c1]

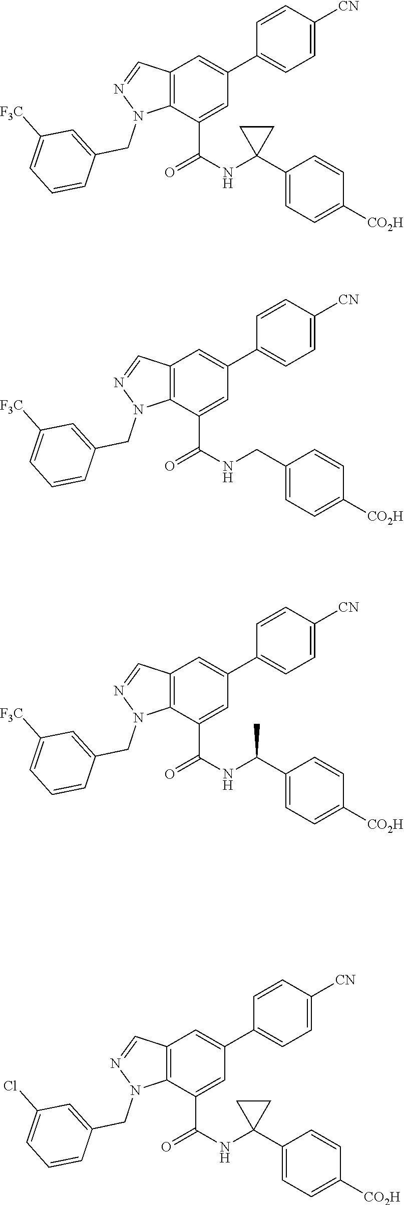 [SMILES: N#Cc1ccc(-c2cc(C(=O)NC3(c4ccc(C(=O)O)cc4)CC3)c3c(cnn3Cc3cccc(C(F)(F)F)c3)c2)cc1 ; N#Cc1ccc(-c2cc(C(=O)NCc3ccc(C(=O)O)cc3)c3c(cnn3Cc3cccc(C(F)(F)F)c3)c2)cc1 ; C[C@H](NC(=O)c1cc(-c2ccc(C#N)cc2)cc2cnn(Cc3cccc(C(F)(F)F)c3)c12)c1ccc(C(=O)O)cc1 ; N#Cc1ccc(-c2cc(C(=O)NC3(c4ccc(C(=O)O)cc4)CC3)c3c(cnn3Cc3cccc(Cl)c3)c2)cc1]

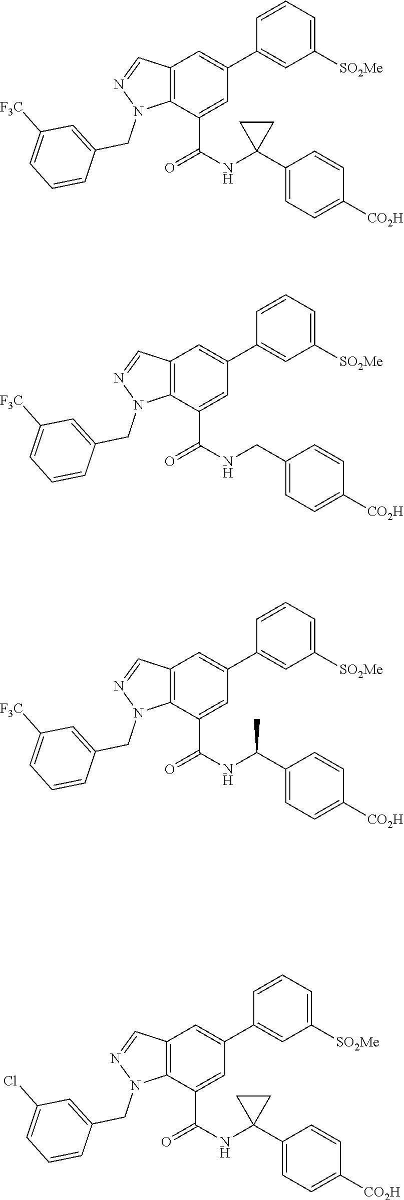 [SMILES: CS(=O)(=O)c1cccc(-c2cc(C(=O)NC3(c4ccc(C(=O)O)cc4)CC3)c3c(cnn3Cc3cccc(C(F)(F)F)c3)c2)c1 ; CS(=O)(=O)c1cccc(-c2cc(C(=O)NCc3ccc(C(=O)O)cc3)c3c(cnn3Cc3cccc(C(F)(F)F)c3)c2)c1 ; C[C@H](NC(=O)c1cc(-c2cccc(S(C)(=O)=O)c2)cc2cnn(Cc3cccc(C(F)(F)F)c3)c12)c1ccc(C(=O)O)cc1 ; CS(=O)(=O)c1cccc(-c2cc(C(=O)NC3(c4ccc(C(=O)O)cc4)CC3)c3c(cnn3Cc3cccc(Cl)c3)c2)c1]

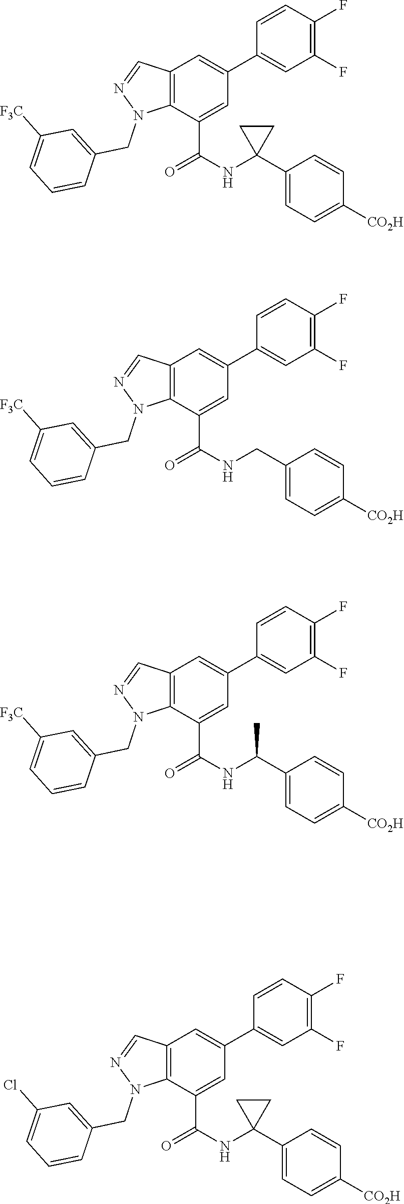 [SMILES: O=C(O)c1ccc(C2(NC(=O)c3cc(-c4ccc(F)c(F)c4)cc4cnn(Cc5cccc(C(F)(F)F)c5)c34)CC2)cc1 ; O=C(O)c1ccc(CNC(=O)c2cc(-c3ccc(F)c(F)c3)cc3cnn(Cc4cccc(C(F)(F)F)c4)c23)cc1 ; C[C@H](NC(=O)c1cc(-c2ccc(F)c(F)c2)cc2cnn(Cc3cccc(C(F)(F)F)c3)c12)c1ccc(C(=O)O)cc1 ; O=C(O)c1ccc(C2(NC(=O)c3cc(-c4ccc(F)c(F)c4)cc4cnn(Cc5cccc(Cl)c5)c34)CC2)cc1]

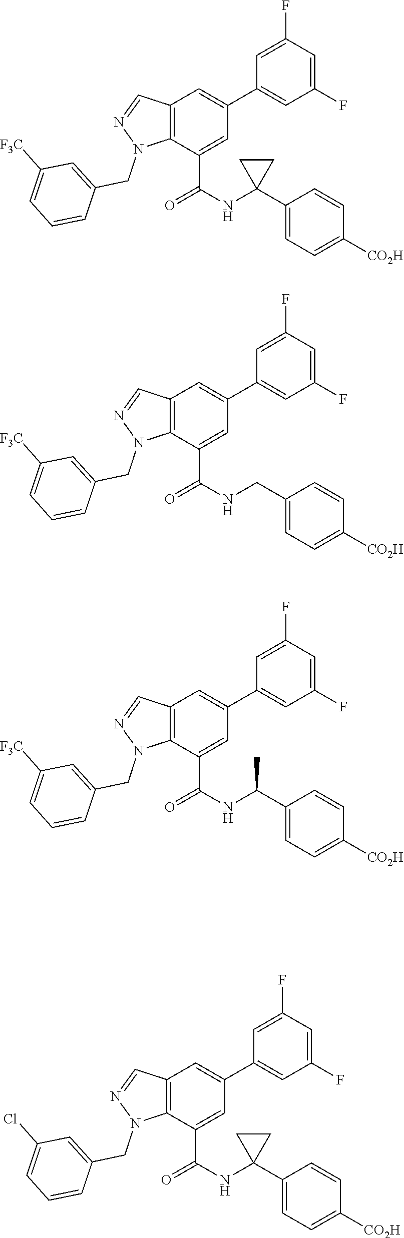 [SMILES: O=C(O)c1ccc(C2(NC(=O)c3cc(-c4cc(F)cc(F)c4)cc4cnn(Cc5cccc(C(F)(F)F)c5)c34)CC2)cc1 ; O=C(O)c1ccc(CNC(=O)c2cc(-c3cc(F)cc(F)c3)cc3cnn(Cc4cccc(C(F)(F)F)c4)c23)cc1 ; C[C@H](NC(=O)c1cc(-c2cc(F)cc(F)c2)cc2cnn(Cc3cccc(C(F)(F)F)c3)c12)c1ccc(C(=O)O)cc1 ; O=C(O)c1ccc(C2(NC(=O)c3cc(-c4cc(F)cc(F)c4)cc4cnn(Cc5cccc(Cl)c5)c34)CC2)cc1]

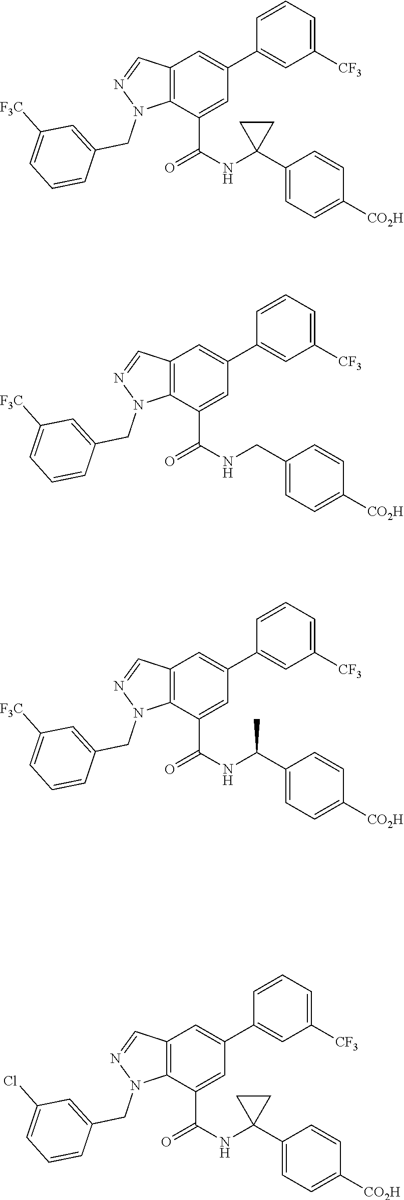 [SMILES: O=C(O)c1ccc(C2(NC(=O)c3cc(-c4cccc(C(F)(F)F)c4)cc4cnn(Cc5cccc(C(F)(F)F)c5)c34)CC2)cc1 ; O=C(O)c1ccc(CNC(=O)c2cc(-c3cccc(C(F)(F)F)c3)cc3cnn(Cc4cccc(C(F)(F)F)c4)c23)cc1 ; C[C@H](NC(=O)c1cc(-c2cccc(C(F)(F)F)c2)cc2cnn(Cc3cccc(C(F)(F)F)c3)c12)c1ccc(C(=O)O)cc1 ; O=C(O)c1ccc(C2(NC(=O)c3cc(-c4cccc(C(F)(F)F)c4)cc4cnn(Cc5cccc(Cl)c5)c34)CC2)cc1]

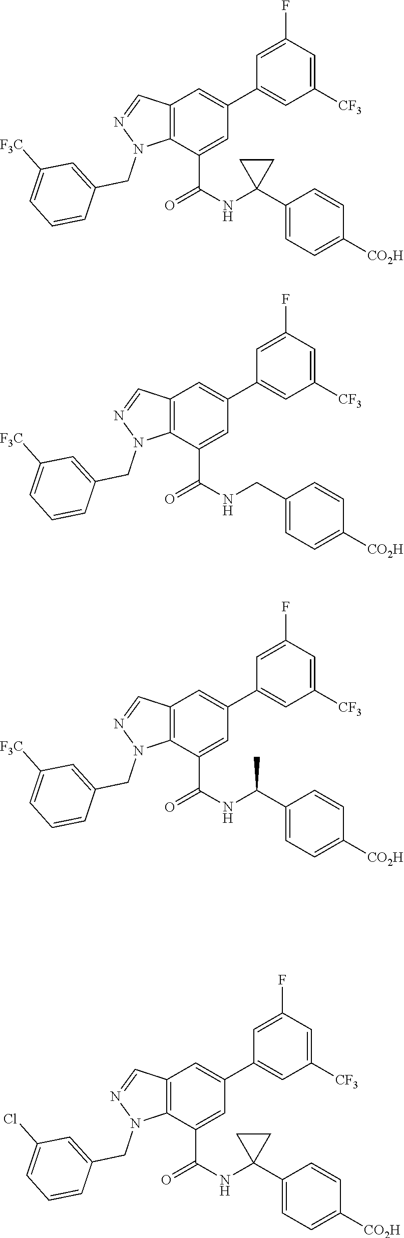 [SMILES: O=C(O)c1ccc(C2(NC(=O)c3cc(-c4cc(F)cc(C(F)(F)F)c4)cc4cnn(Cc5cccc(C(F)(F)F)c5)c34)CC2)cc1 ; O=C(O)c1ccc(CNC(=O)c2cc(-c3cc(F)cc(C(F)(F)F)c3)cc3cnn(Cc4cccc(C(F)(F)F)c4)c23)cc1 ; C[C@H](NC(=O)c1cc(-c2cc(F)cc(C(F)(F)F)c2)cc2cnn(Cc3cccc(C(F)(F)F)c3)c12)c1ccc(C(=O)O)cc1 ; O=C(O)c1ccc(C2(NC(=O)c3cc(-c4cc(F)cc(C(F)(F)F)c4)cc4cnn(Cc5cccc(Cl)c5)c34)CC2)cc1]

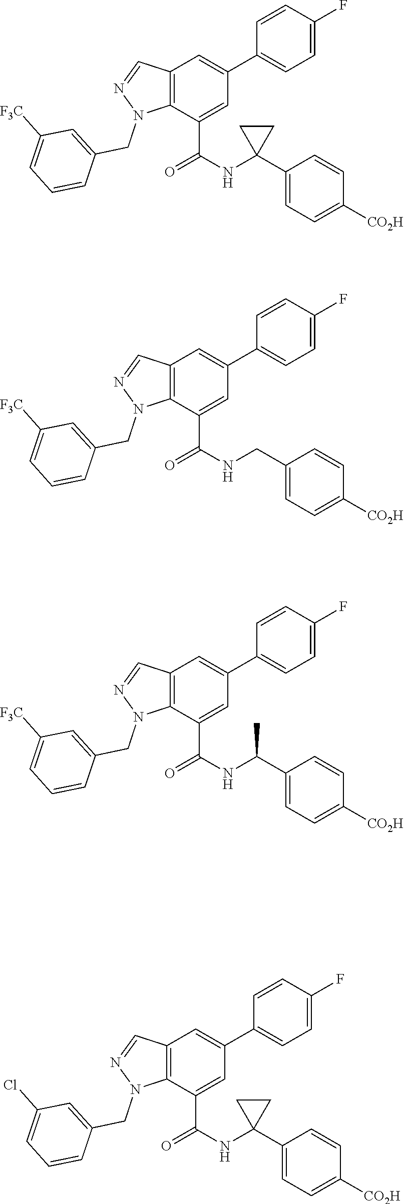 [SMILES: O=C(O)c1ccc(C2(NC(=O)c3cc(-c4ccc(F)cc4)cc4cnn(Cc5cccc(C(F)(F)F)c5)c34)CC2)cc1 ; O=C(O)c1ccc(CNC(=O)c2cc(-c3ccc(F)cc3)cc3cnn(Cc4cccc(C(F)(F)F)c4)c23)cc1 ; C[C@H](NC(=O)c1cc(-c2ccc(F)cc2)cc2cnn(Cc3cccc(C(F)(F)F)c3)c12)c1ccc(C(=O)O)cc1 ; O=C(O)c1ccc(C2(NC(=O)c3cc(-c4ccc(F)cc4)cc4cnn(Cc5cccc(Cl)c5)c34)CC2)cc1]

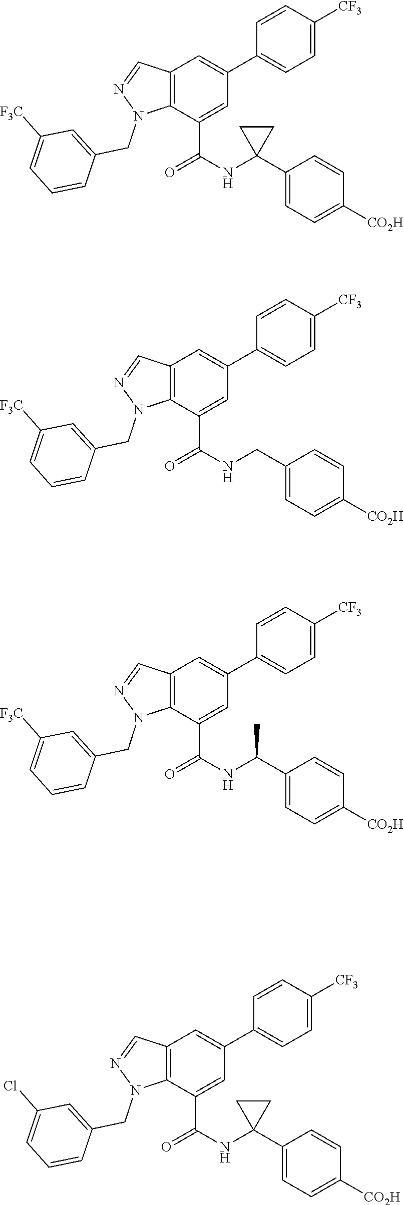 [SMILES: O=C(O)c1ccc(C2(NC(=O)c3cc(-c4ccc(C(F)(F)F)cc4)cc4cnn(Cc5cccc(C(F)(F)F)c5)c34)CC2)cc1 ; O=C(O)c1ccc(CNC(=O)c2cc(-c3ccc(C(F)(F)F)cc3)cc3cnn(Cc4cccc(C(F)(F)F)c4)c23)cc1 ; C[C@H](NC(=O)c1cc(-c2ccc(C(F)(F)F)cc2)cc2cnn(Cc3cccc(C(F)(F)F)c3)c12)c1ccc(C(=O)O)cc1 ; O=C(O)c1ccc(C2(NC(=O)c3cc(-c4ccc(C(F)(F)F)cc4)cc4cnn(Cc5cccc(Cl)c5)c34)CC2)cc1]

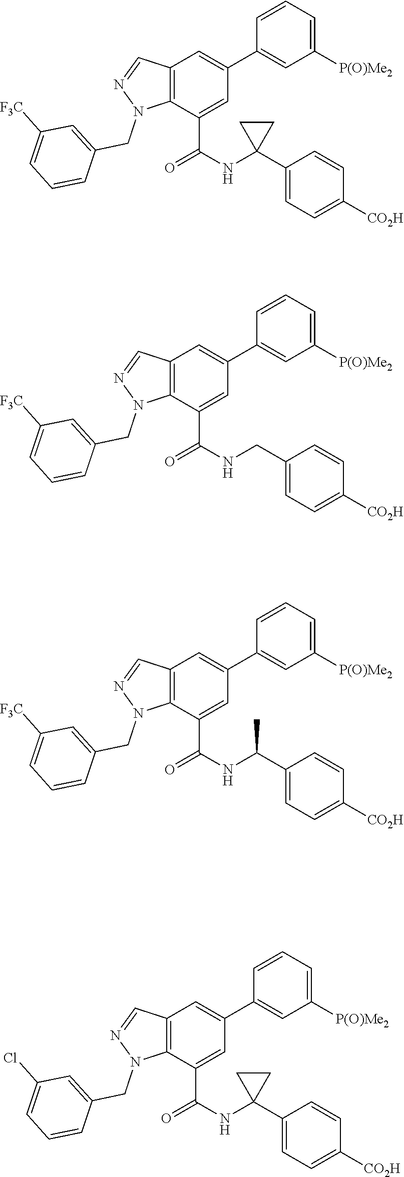

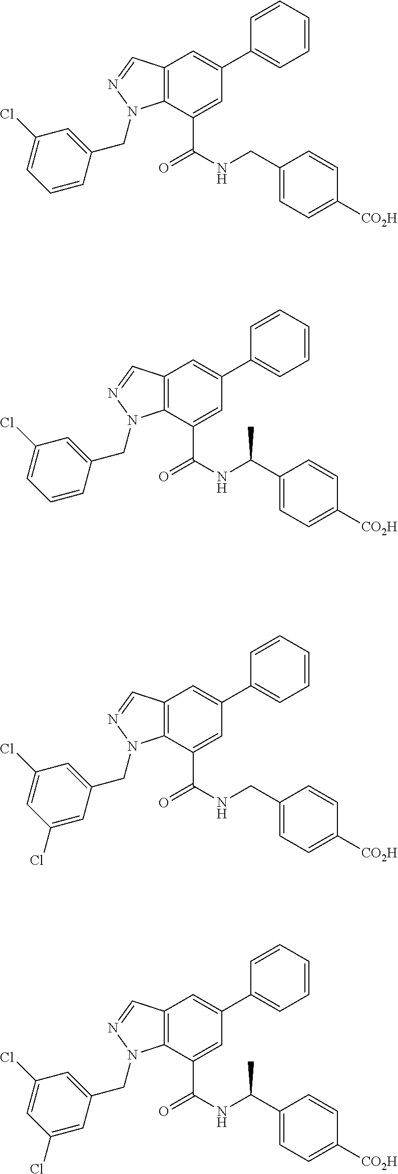 [SMILES: O=C(O)c1ccc(CNC(=O)c2cc(-c3ccccc3)cc3cnn(Cc4cccc(Cl)c4)c23)cc1 ; C[C@H](NC(=O)c1cc(-c2ccccc2)cc2cnn(Cc3cccc(Cl)c3)c12)c1ccc(C(=O)O)cc1 ; O=C(O)c1ccc(CNC(=O)c2cc(-c3ccccc3)cc3cnn(Cc4cc(Cl)cc(Cl)c4)c23)cc1 ; C[C@H](NC(=O)c1cc(-c2ccccc2)cc2cnn(Cc3cc(Cl)cc(Cl)c3)c12)c1ccc(C(=O)O)cc1]

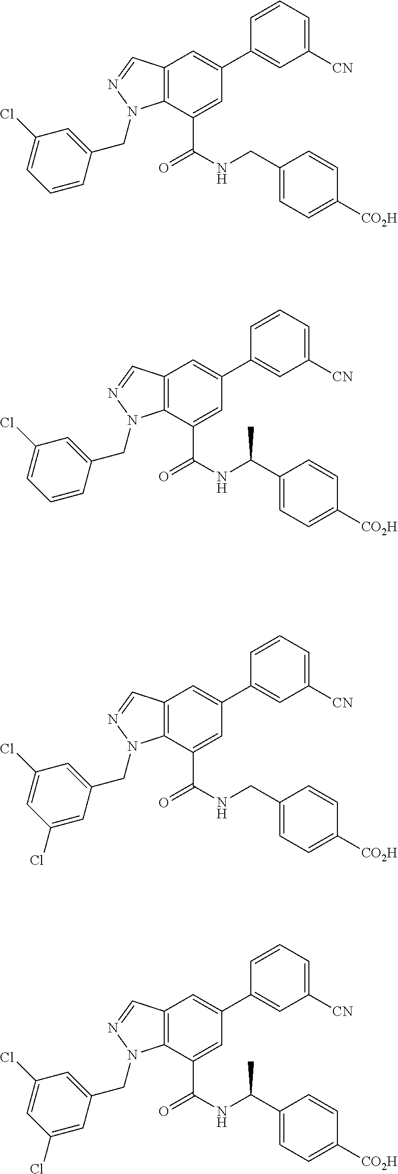 [SMILES: N#Cc1cccc(-c2cc(C(=O)NCc3ccc(C(=O)O)cc3)c3c(cnn3Cc3cccc(Cl)c3)c2)c1 ; C[C@H](NC(=O)c1cc(-c2cccc(C#N)c2)cc2cnn(Cc3cccc(Cl)c3)c12)c1ccc(C(=O)O)cc1 ; N#Cc1cccc(-c2cc(C(=O)NCc3ccc(C(=O)O)cc3)c3c(cnn3Cc3cc(Cl)cc(Cl)c3)c2)c1 ; C[C@H](NC(=O)c1cc(-c2cccc(C#N)c2)cc2cnn(Cc3cc(Cl)cc(Cl)c3)c12)c1ccc(C(=O)O)cc1]

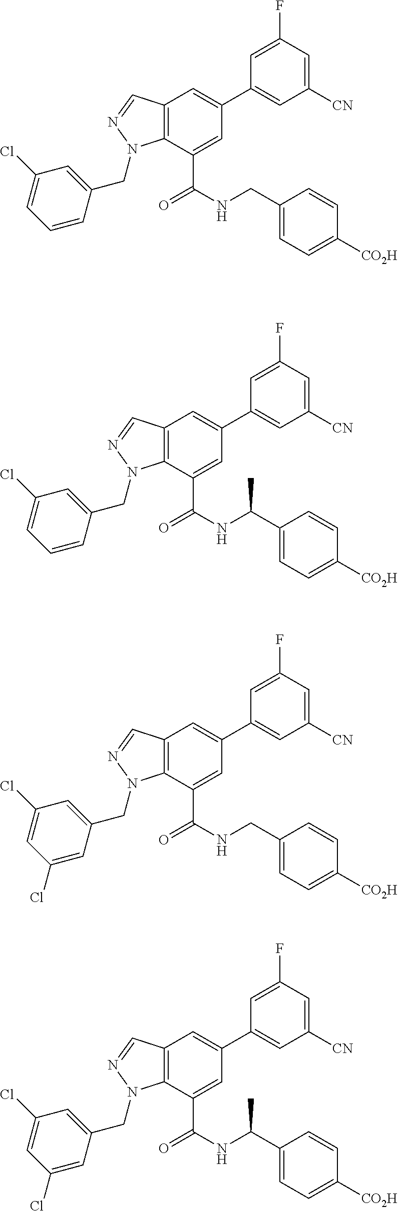 [SMILES: N#Cc1cc(F)cc(-c2cc(C(=O)NCc3ccc(C(=O)O)cc3)c3c(cnn3Cc3cccc(Cl)c3)c2)c1 ; C[C@H](NC(=O)c1cc(-c2cc(F)cc(C#N)c2)cc2cnn(Cc3cccc(Cl)c3)c12)c1ccc(C(=O)O)cc1 ; N#Cc1cc(F)cc(-c2cc(C(=O)NCc3ccc(C(=O)O)cc3)c3c(cnn3Cc3cc(Cl)cc(Cl)c3)c2)c1 ; C[C@H](NC(=O)c1cc(-c2cc(F)cc(C#N)c2)cc2cnn(Cc3cc(Cl)cc(Cl)c3)c12)c1ccc(C(=O)O)cc1]

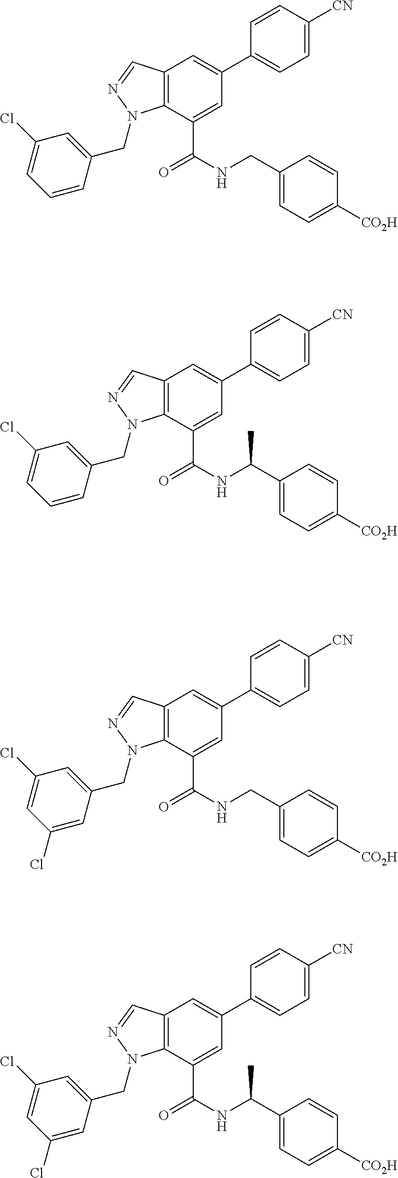 [SMILES: N#Cc1ccc(-c2cc(C(=O)NCc3ccc(C(=O)O)cc3)c3c(cnn3Cc3cccc(Cl)c3)c2)cc1 ; C[C@H](NC(=O)c1cc(-c2ccc(C#N)cc2)cc2cnn(Cc3cccc(Cl)c3)c12)c1ccc(C(=O)O)cc1 ; N#Cc1ccc(-c2cc(C(=O)NCc3ccc(C(=O)O)cc3)c3c(cnn3Cc3cc(Cl)cc(Cl)c3)c2)cc1 ; C[C@H](NC(=O)c1cc(-c2ccc(C#N)cc2)cc2cnn(Cc3cc(Cl)cc(Cl)c3)c12)c1ccc(C(=O)O)cc1]

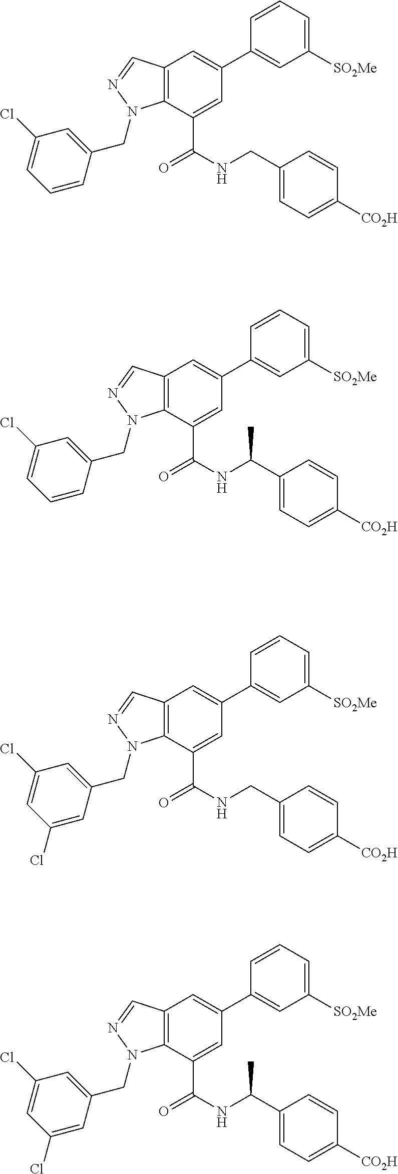 [SMILES: CS(=O)(=O)c1cccc(-c2cc(C(=O)NCc3ccc(C(=O)O)cc3)c3c(cnn3Cc3cccc(Cl)c3)c2)c1 ; C[C@H](NC(=O)c1cc(-c2cccc(S(C)(=O)=O)c2)cc2cnn(Cc3cccc(Cl)c3)c12)c1ccc(C(=O)O)cc1 ; CS(=O)(=O)c1cccc(-c2cc(C(=O)NCc3ccc(C(=O)O)cc3)c3c(cnn3Cc3cc(Cl)cc(Cl)c3)c2)c1 ; C[C@H](NC(=O)c1cc(-c2cccc(S(C)(=O)=O)c2)cc2cnn(Cc3cc(Cl)cc(Cl)c3)c12)c1ccc(C(=O)O)cc1]

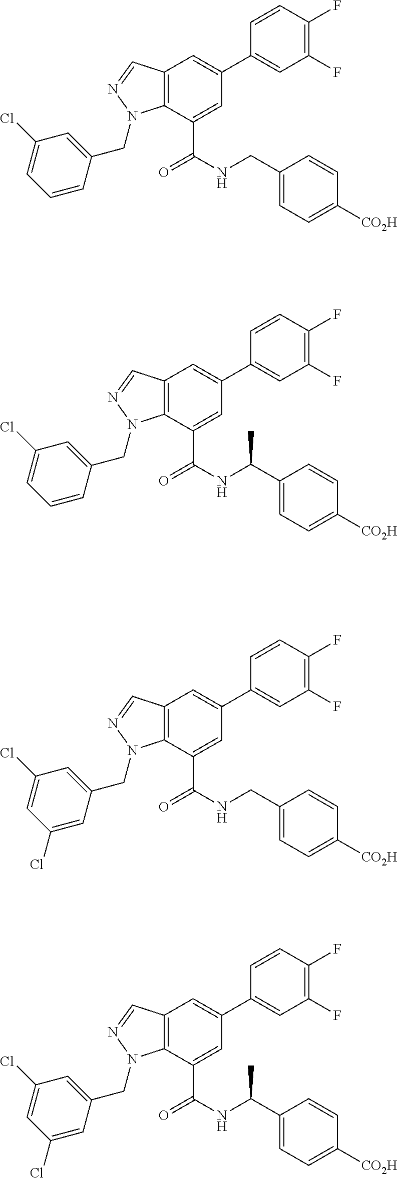 [SMILES: O=C(O)c1ccc(CNC(=O)c2cc(-c3ccc(F)c(F)c3)cc3cnn(Cc4cccc(Cl)c4)c23)cc1 ; C[C@H](NC(=O)c1cc(-c2ccc(F)c(F)c2)cc2cnn(Cc3cccc(Cl)c3)c12)c1ccc(C(=O)O)cc1 ; O=C(O)c1ccc(CNC(=O)c2cc(-c3ccc(F)c(F)c3)cc3cnn(Cc4cc(Cl)cc(Cl)c4)c23)cc1 ; C[C@H](NC(=O)c1cc(-c2ccc(F)c(F)c2)cc2cnn(Cc3cc(Cl)cc(Cl)c3)c12)c1ccc(C(=O)O)cc1]

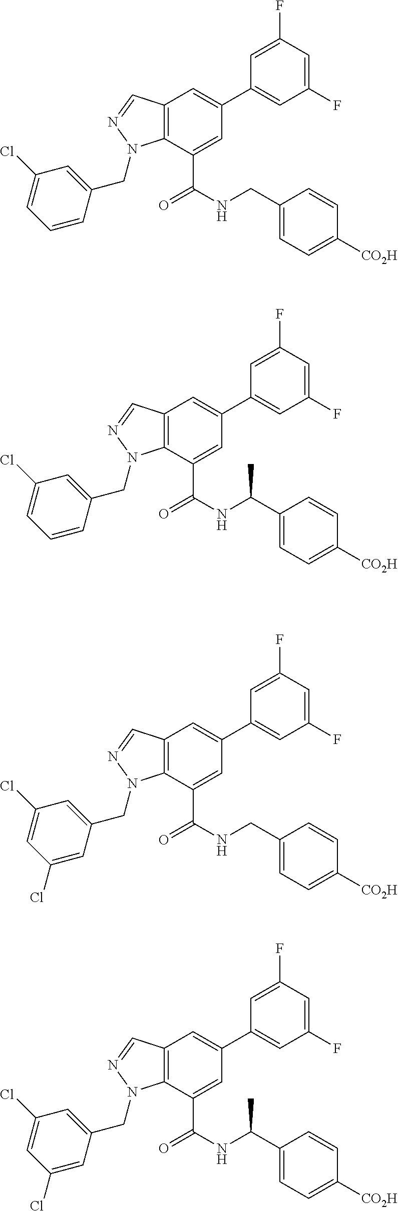 [SMILES: O=C(O)c1ccc(CNC(=O)c2cc(-c3cc(F)cc(F)c3)cc3cnn(Cc4cccc(Cl)c4)c23)cc1 ; C[C@H](NC(=O)c1cc(-c2cc(F)cc(F)c2)cc2cnn(Cc3cccc(Cl)c3)c12)c1ccc(C(=O)O)cc1 ; O=C(O)c1ccc(CNC(=O)c2cc(-c3cc(F)cc(F)c3)cc3cnn(Cc4cc(Cl)cc(Cl)c4)c23)cc1 ; C[C@H](NC(=O)c1cc(-c2cc(F)cc(F)c2)cc2cnn(Cc3cc(Cl)cc(Cl)c3)c12)c1ccc(C(=O)O)cc1]

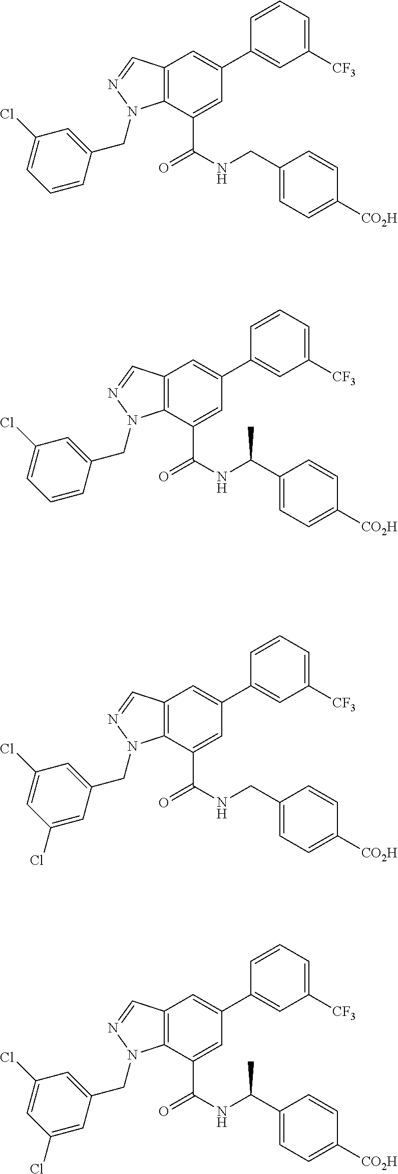 [SMILES: O=C(O)c1ccc(CNC(=O)c2cc(-c3cccc(C(F)(F)F)c3)cc3cnn(Cc4cccc(Cl)c4)c23)cc1 ; C[C@H](NC(=O)c1cc(-c2cccc(C(F)(F)F)c2)cc2cnn(Cc3cccc(Cl)c3)c12)c1ccc(C(=O)O)cc1 ; O=C(O)c1ccc(CNC(=O)c2cc(-c3cccc(C(F)(F)F)c3)cc3cnn(Cc4cc(Cl)cc(Cl)c4)c23)cc1 ; C[C@H](NC(=O)c1cc(-c2cccc(C(F)(F)F)c2)cc2cnn(Cc3cc(Cl)cc(Cl)c3)c12)c1ccc(C(=O)O)cc1]

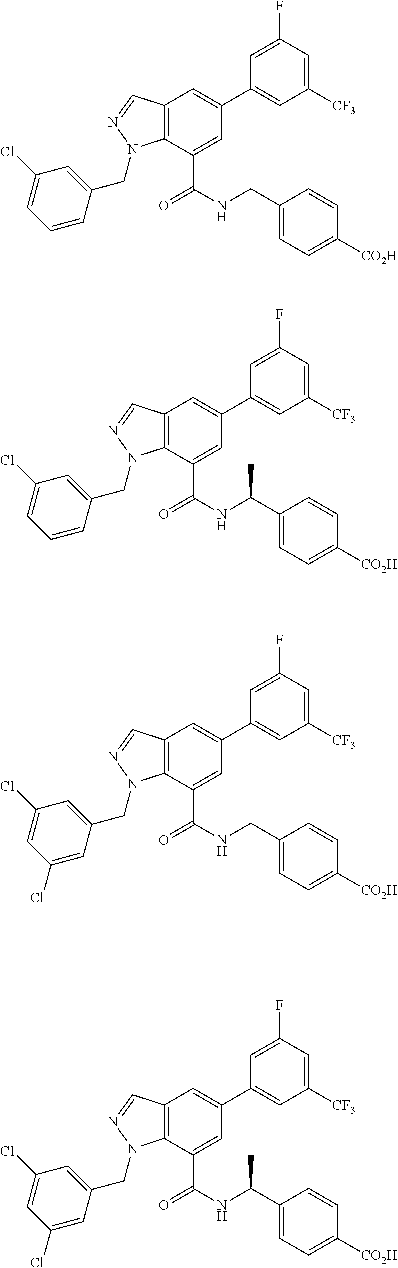 [SMILES: O=C(O)c1ccc(CNC(=O)c2cc(-c3cc(F)cc(C(F)(F)F)c3)cc3cnn(Cc4cccc(Cl)c4)c23)cc1 ; C[C@H](NC(=O)c1cc(-c2cc(F)cc(C(F)(F)F)c2)cc2cnn(Cc3cccc(Cl)c3)c12)c1ccc(C(=O)O)cc1 ; O=C(O)c1ccc(CNC(=O)c2cc(-c3cc(F)cc(C(F)(F)F)c3)cc3cnn(Cc4cc(Cl)cc(Cl)c4)c23)cc1 ; C[C@H](NC(=O)c1cc(-c2cc(F)cc(C(F)(F)F)c2)cc2cnn(Cc3cc(Cl)cc(Cl)c3)c12)c1ccc(C(=O)O)cc1]

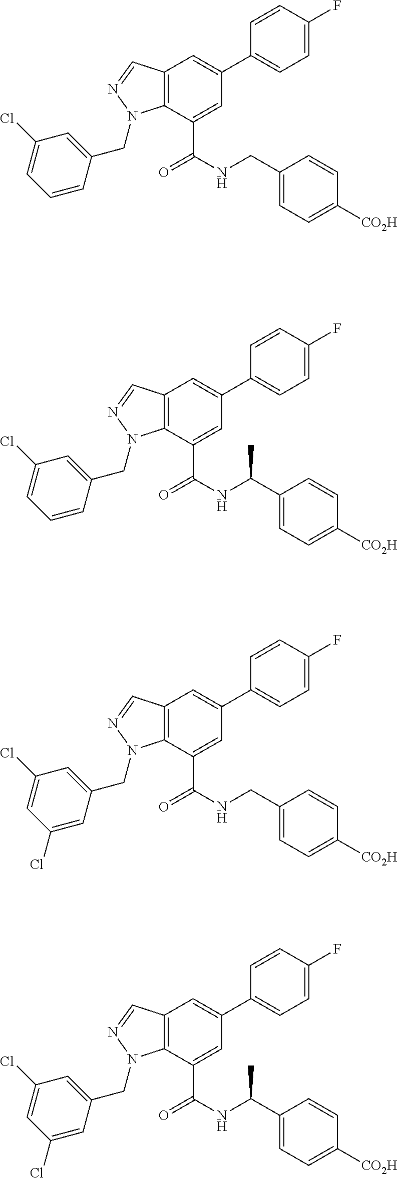 [SMILES: O=C(O)c1ccc(CNC(=O)c2cc(-c3ccc(F)cc3)cc3cnn(Cc4cccc(Cl)c4)c23)cc1 ; C[C@H](NC(=O)c1cc(-c2ccc(F)cc2)cc2cnn(Cc3cccc(Cl)c3)c12)c1ccc(C(=O)O)cc1 ; O=C(O)c1ccc(CNC(=O)c2cc(-c3ccc(F)cc3)cc3cnn(Cc4cc(Cl)cc(Cl)c4)c23)cc1 ; C[C@H](NC(=O)c1cc(-c2ccc(F)cc2)cc2cnn(Cc3cc(Cl)cc(Cl)c3)c12)c1ccc(C(=O)O)cc1]

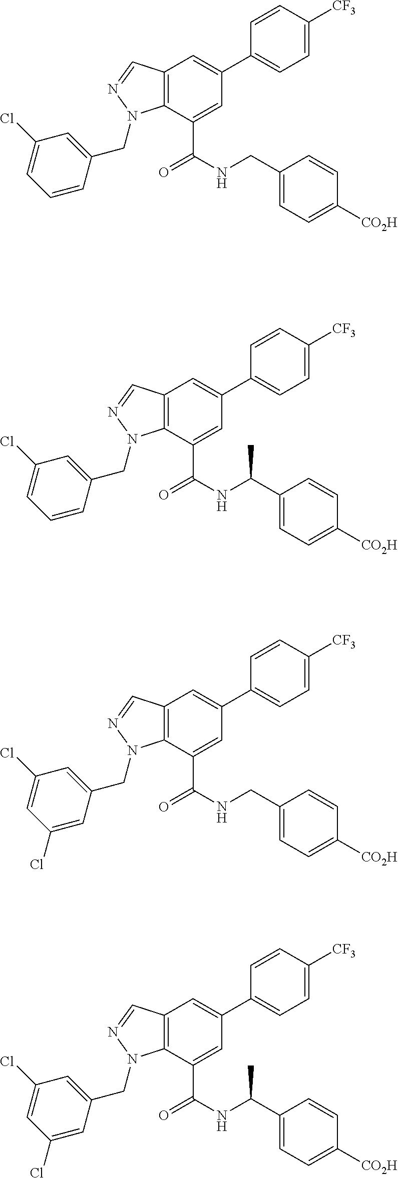 [SMILES: O=C(O)c1ccc(CNC(=O)c2cc(-c3ccc(C(F)(F)F)cc3)cc3cnn(Cc4cccc(Cl)c4)c23)cc1 ; C[C@H](NC(=O)c1cc(-c2ccc(C(F)(F)F)cc2)cc2cnn(Cc3cccc(Cl)c3)c12)c1ccc(C(=O)O)cc1 ; O=C(O)c1ccc(CNC(=O)c2cc(-c3ccc(C(F)(F)F)cc3)cc3cnn(Cc4cc(Cl)cc(Cl)c4)c23)cc1 ; C[C@H](NC(=O)c1cc(-c2ccc(C(F)(F)F)cc2)cc2cnn(Cc3cc(Cl)cc(Cl)c3)c12)c1ccc(C(=O)O)cc1]

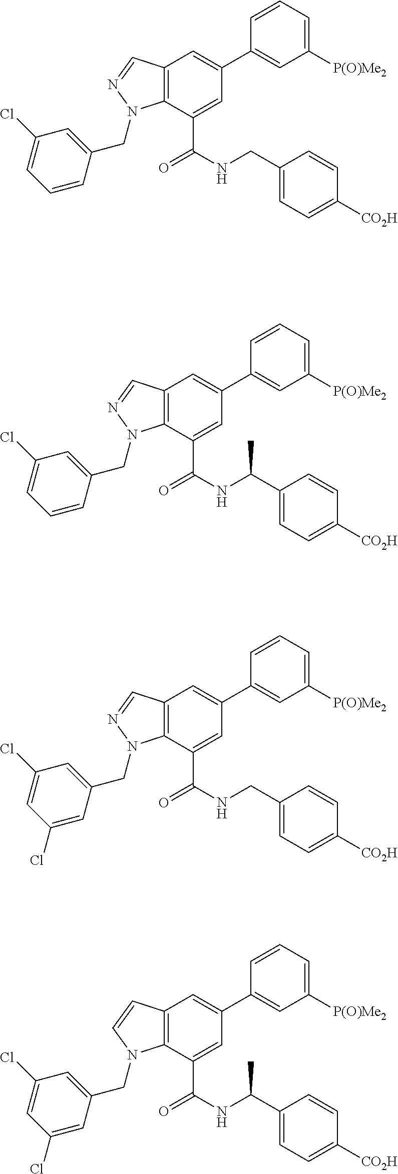

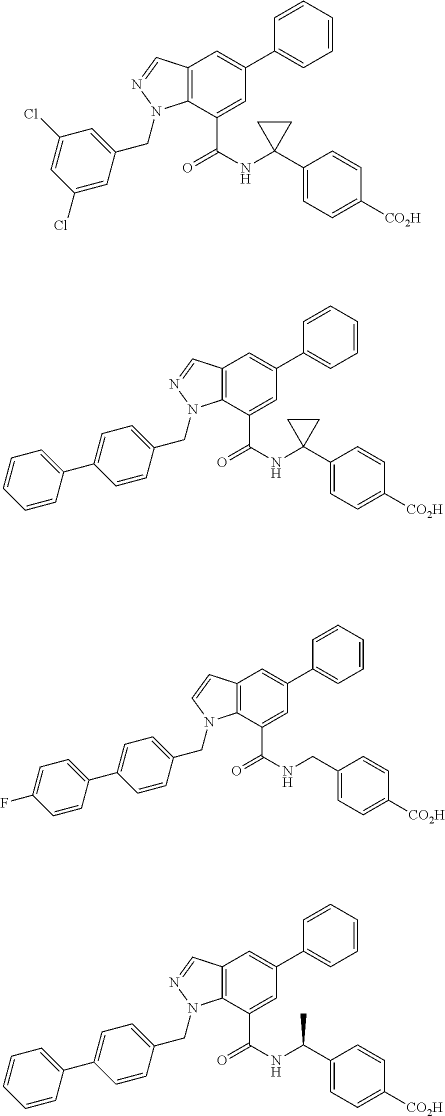 [SMILES: O=C(O)c1ccc(C2(NC(=O)c3cc(-c4ccccc4)cc4cnn(Cc5cc(Cl)cc(Cl)c5)c34)CC2)cc1 ; O=C(O)c1ccc(C2(NC(=O)c3cc(-c4ccccc4)cc4cnn(Cc5ccc(-c6ccccc6)cc5)c34)CC2)cc1 ; O=C(O)c1ccc(CNC(=O)c2cc(-c3ccccc3)cc3ccn(Cc4ccc(-c5ccc(F)cc5)cc4)c23)cc1 ; C[C@H](NC(=O)c1cc(-c2ccccc2)cc2cnn(Cc3ccc(-c4ccccc4)cc3)c12)c1ccc(C(=O)O)cc1]

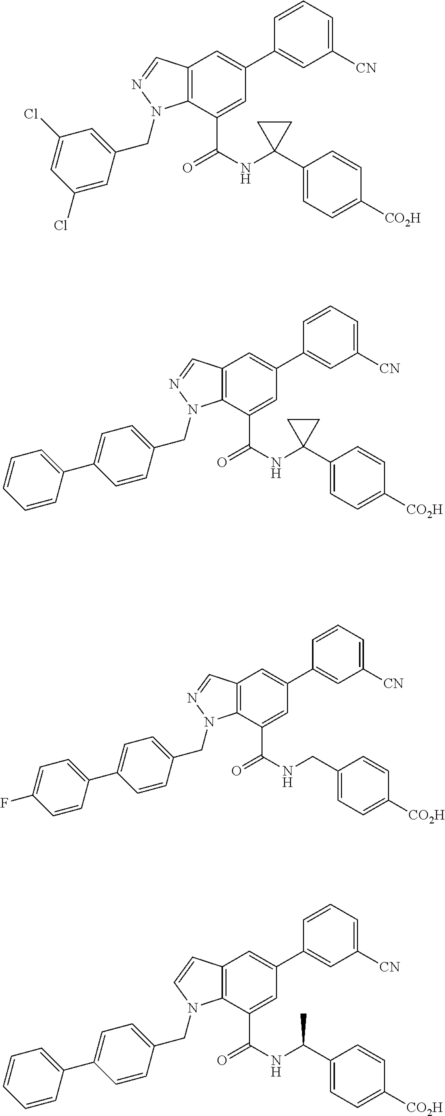 [SMILES: N#Cc1cccc(-c2cc(C(=O)NC3(c4ccc(C(=O)O)cc4)CC3)c3c(cnn3Cc3cc(Cl)cc(Cl)c3)c2)c1 ; N#Cc1cccc(-c2cc(C(=O)NC3(c4ccc(C(=O)O)cc4)CC3)c3c(cnn3Cc3ccc(-c4ccccc4)cc3)c2)c1 ; N#Cc1cccc(-c2cc(C(=O)NCc3ccc(C(=O)O)cc3)c3c(cnn3Cc3ccc(-c4ccc(F)cc4)cc3)c2)c1 ; C[C@H](NC(=O)c1cc(-c2cccc(C#N)c2)cc2ccn(Cc3ccc(-c4ccccc4)cc3)c12)c1ccc(C(=O)O)cc1]

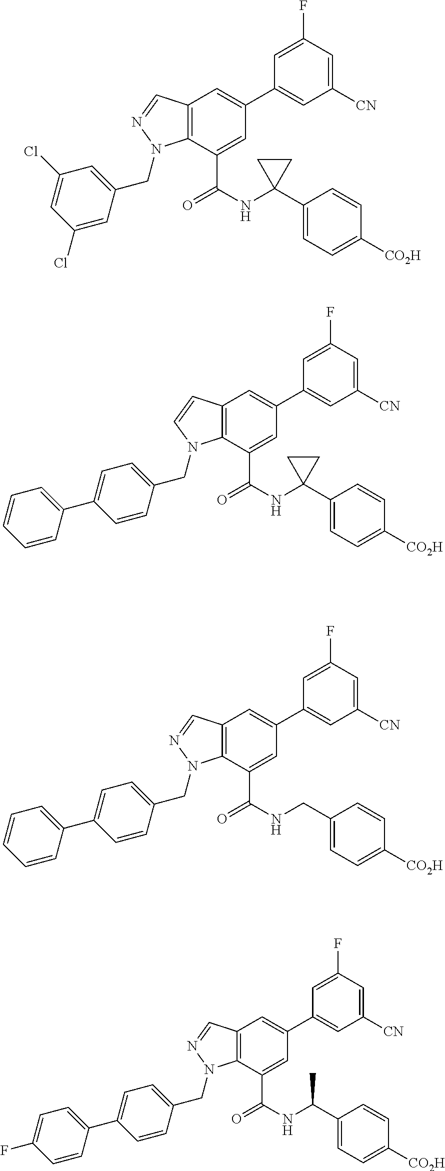 [SMILES: N#Cc1cc(F)cc(-c2cc(C(=O)NC3(c4ccc(C(=O)O)cc4)CC3)c3c(cnn3Cc3cc(Cl)cc(Cl)c3)c2)c1 ; N#Cc1cc(F)cc(-c2cc(C(=O)NC3(c4ccc(C(=O)O)cc4)CC3)c3c(ccn3Cc3ccc(-c4ccccc4)cc3)c2)c1 ; N#Cc1cc(F)cc(-c2cc(C(=O)NCc3ccc(C(=O)O)cc3)c3c(cnn3Cc3ccc(-c4ccccc4)cc3)c2)c1 ; C[C@H](NC(=O)c1cc(-c2cc(F)cc(C#N)c2)cc2cnn(Cc3ccc(-c4ccc(F)cc4)cc3)c12)c1ccc(C(=O)O)cc1]

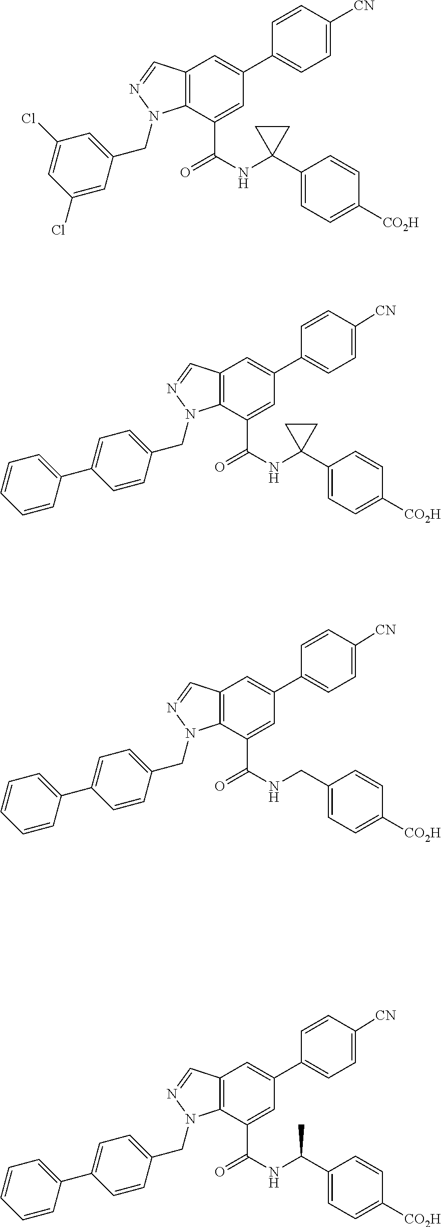 [SMILES: N#Cc1ccc(-c2cc(C(=O)NC3(c4ccc(C(=O)O)cc4)CC3)c3c(cnn3Cc3cc(Cl)cc(Cl)c3)c2)cc1 ; N#Cc1ccc(-c2cc(C(=O)NC3(c4ccc(C(=O)O)cc4)CC3)c3c(cnn3Cc3ccc(-c4ccccc4)cc3)c2)cc1 ; N#Cc1ccc(-c2cc(C(=O)NCc3ccc(C(=O)O)cc3)c3c(cnn3Cc3ccc(-c4ccccc4)cc3)c2)cc1 ; C[C@H](NC(=O)c1cc(-c2ccc(C#N)cc2)cc2cnn(Cc3ccc(-c4ccccc4)cc3)c12)c1ccc(C(=O)O)cc1]

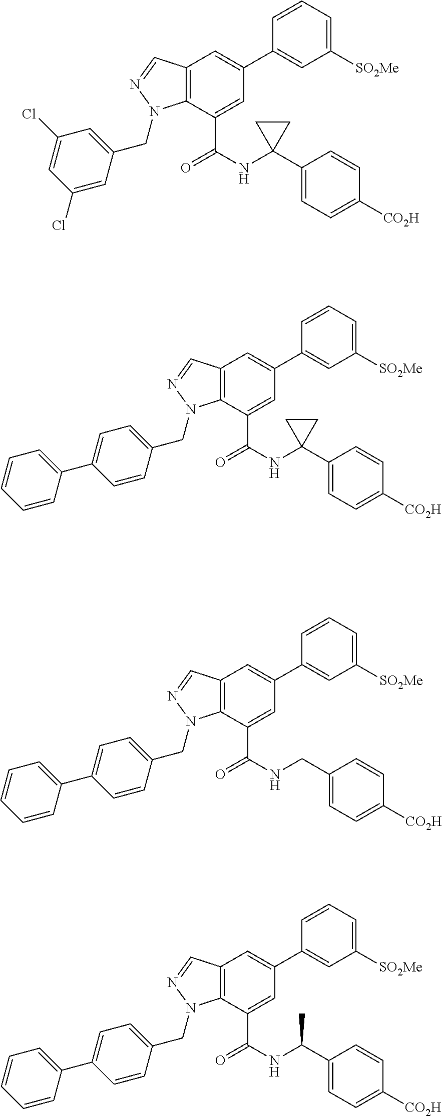 [SMILES: CS(=O)(=O)c1cccc(-c2cc(C(=O)NC3(c4ccc(C(=O)O)cc4)CC3)c3c(cnn3Cc3cc(Cl)cc(Cl)c3)c2)c1 ; CS(=O)(=O)c1cccc(-c2cc(C(=O)NC3(c4ccc(C(=O)O)cc4)CC3)c3c(cnn3Cc3ccc(-c4ccccc4)cc3)c2)c1 ; CS(=O)(=O)c1cccc(-c2cc(C(=O)NCc3ccc(C(=O)O)cc3)c3c(cnn3Cc3ccc(-c4ccccc4)cc3)c2)c1 ; C[C@H](NC(=O)c1cc(-c2cccc(S(C)(=O)=O)c2)cc2cnn(Cc3ccc(-c4ccccc4)cc3)c12)c1ccc(C(=O)O)cc1]

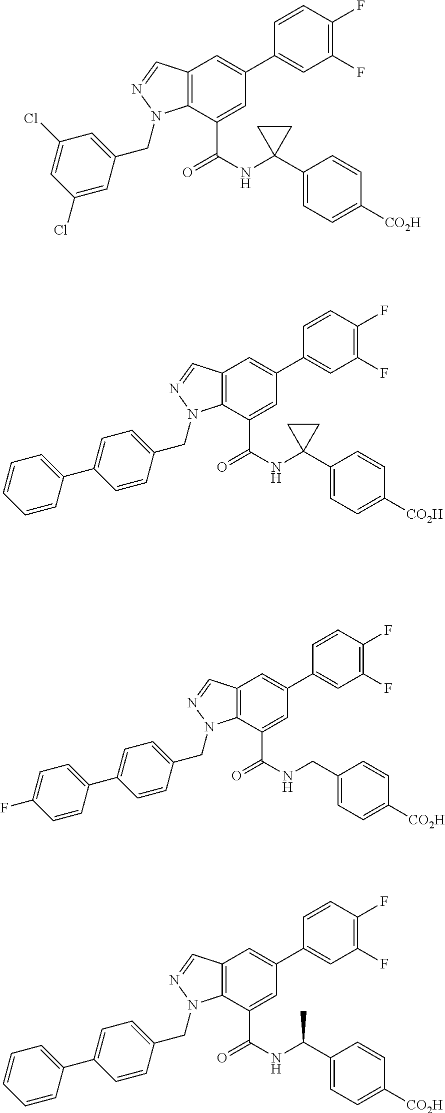 [SMILES: O=C(O)c1ccc(C2(NC(=O)c3cc(-c4ccc(F)c(F)c4)cc4cnn(Cc5cc(Cl)cc(Cl)c5)c34)CC2)cc1 ; O=C(O)c1ccc(C2(NC(=O)c3cc(-c4ccc(F)c(F)c4)cc4cnn(Cc5ccc(-c6ccccc6)cc5)c34)CC2)cc1 ; O=C(O)c1ccc(CNC(=O)c2cc(-c3ccc(F)c(F)c3)cc3cnn(Cc4ccc(-c5ccc(F)cc5)cc4)c23)cc1 ; C[C@H](NC(=O)c1cc(-c2ccc(F)c(F)c2)cc2cnn(Cc3ccc(-c4ccccc4)cc3)c12)c1ccc(C(=O)O)cc1]

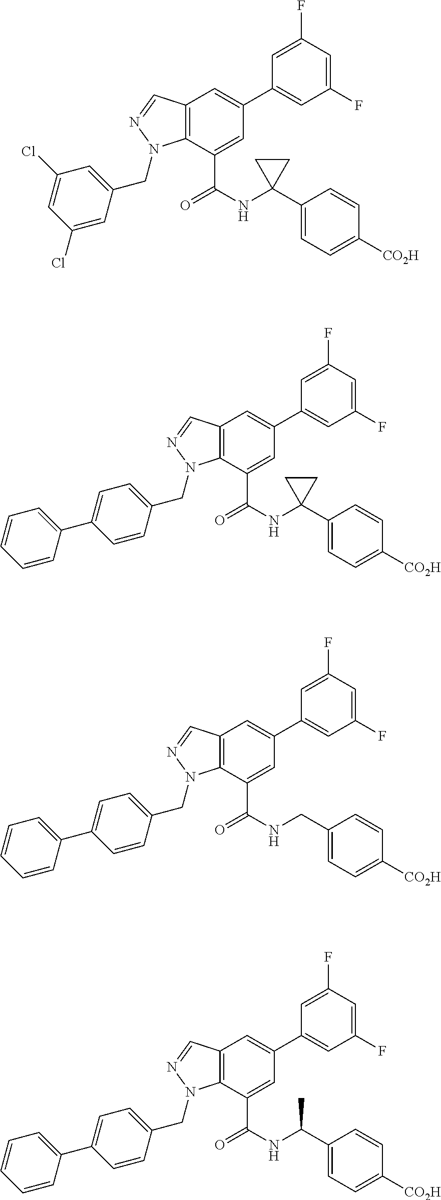 [SMILES: O=C(O)c1ccc(C2(NC(=O)c3cc(-c4cc(F)cc(F)c4)cc4cnn(Cc5cc(Cl)cc(Cl)c5)c34)CC2)cc1 ; O=C(O)c1ccc(C2(NC(=O)c3cc(-c4cc(F)cc(F)c4)cc4cnn(Cc5ccc(-c6ccccc6)cc5)c34)CC2)cc1 ; O=C(O)c1ccc(CNC(=O)c2cc(-c3cc(F)cc(F)c3)cc3cnn(Cc4ccc(-c5ccccc5)cc4)c23)cc1 ; C[C@H](NC(=O)c1cc(-c2cc(F)cc(F)c2)cc2cnn(Cc3ccc(-c4ccccc4)cc3)c12)c1ccc(C(=O)O)cc1]

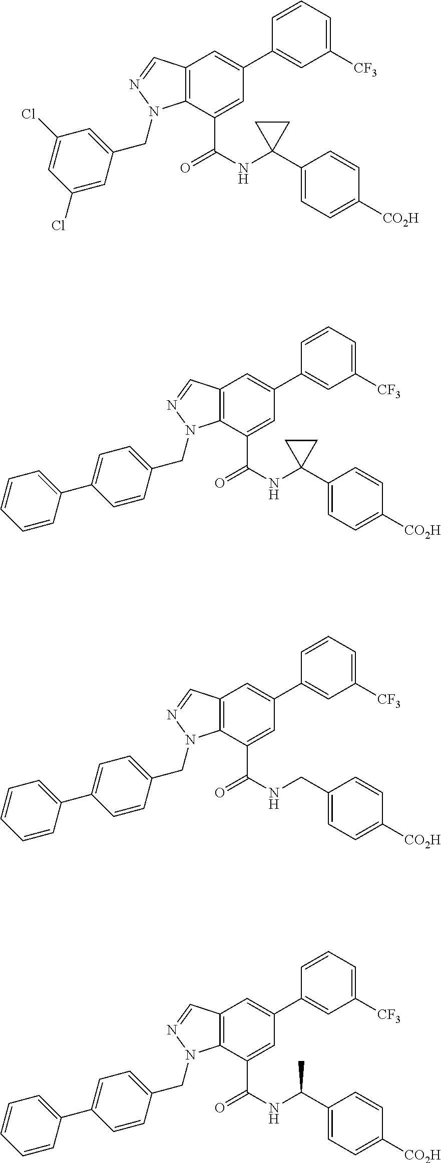 [SMILES: O=C(O)c1ccc(C2(NC(=O)c3cc(-c4cccc(C(F)(F)F)c4)cc4cnn(Cc5cc(Cl)cc(Cl)c5)c34)CC2)cc1 ; O=C(O)c1ccc(C2(NC(=O)c3cc(-c4cccc(C(F)(F)F)c4)cc4cnn(Cc5ccc(-c6ccccc6)cc5)c34)CC2)cc1 ; O=C(O)c1ccc(CNC(=O)c2cc(-c3cccc(C(F)(F)F)c3)cc3cnn(Cc4ccc(-c5ccccc5)cc4)c23)cc1 ; C[C@H](NC(=O)c1cc(-c2cccc(C(F)(F)F)c2)cc2cnn(Cc3ccc(-c4ccccc4)cc3)c12)c1ccc(C(=O)O)cc1]

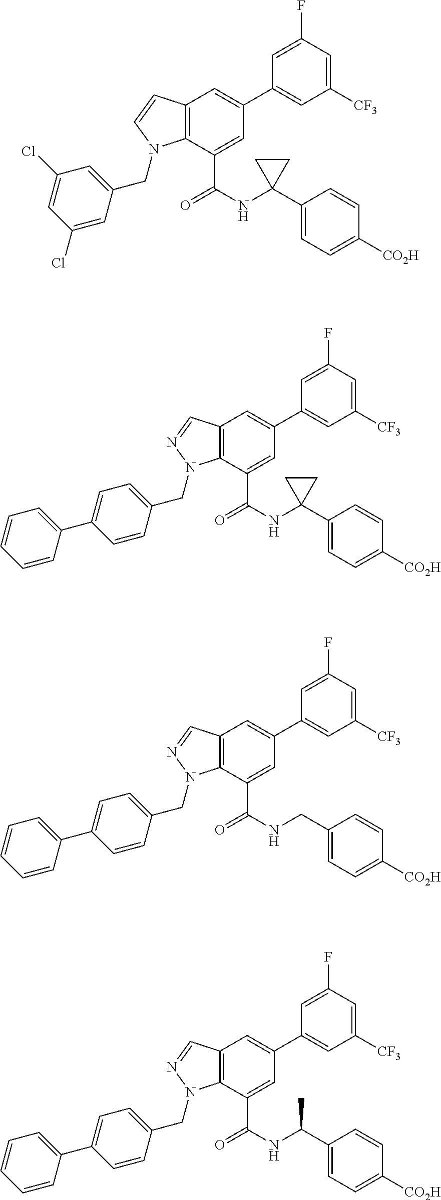 [SMILES: O=C(O)c1ccc(C2(NC(=O)c3cc(-c4cc(F)cc(C(F)(F)F)c4)cc4ccn(Cc5cc(Cl)cc(Cl)c5)c34)CC2)cc1 ; O=C(O)c1ccc(C2(NC(=O)c3cc(-c4cc(F)cc(C(F)(F)F)c4)cc4cnn(Cc5ccc(-c6ccccc6)cc5)c34)CC2)cc1 ; O=C(O)c1ccc(CNC(=O)c2cc(-c3cc(F)cc(C(F)(F)F)c3)cc3cnn(Cc4ccc(-c5ccccc5)cc4)c23)cc1 ; C[C@H](NC(=O)c1cc(-c2cc(F)cc(C(F)(F)F)c2)cc2cnn(Cc3ccc(-c4ccccc4)cc3)c12)c1ccc(C(=O)O)cc1]

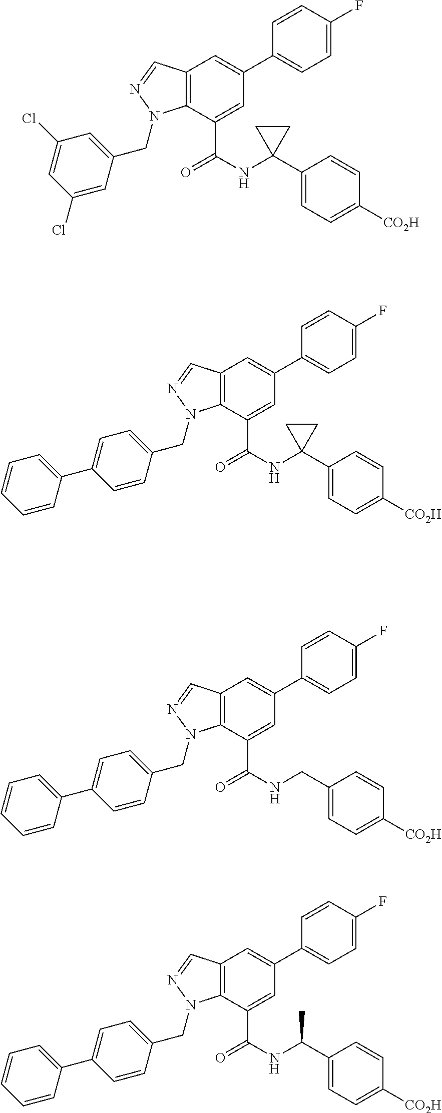 [SMILES: O=C(O)c1ccc(C2(NC(=O)c3cc(-c4ccc(F)cc4)cc4cnn(Cc5cc(Cl)cc(Cl)c5)c34)CC2)cc1 ; O=C(O)c1ccc(C2(NC(=O)c3cc(-c4ccc(F)cc4)cc4cnn(Cc5ccc(-c6ccccc6)cc5)c34)CC2)cc1 ; O=C(O)c1ccc(CNC(=O)c2cc(-c3ccc(F)cc3)cc3cnn(Cc4ccc(-c5ccccc5)cc4)c23)cc1 ; C[C@H](NC(=O)c1cc(-c2ccc(F)cc2)cc2cnn(Cc3ccc(-c4ccccc4)cc3)c12)c1ccc(C(=O)O)cc1]

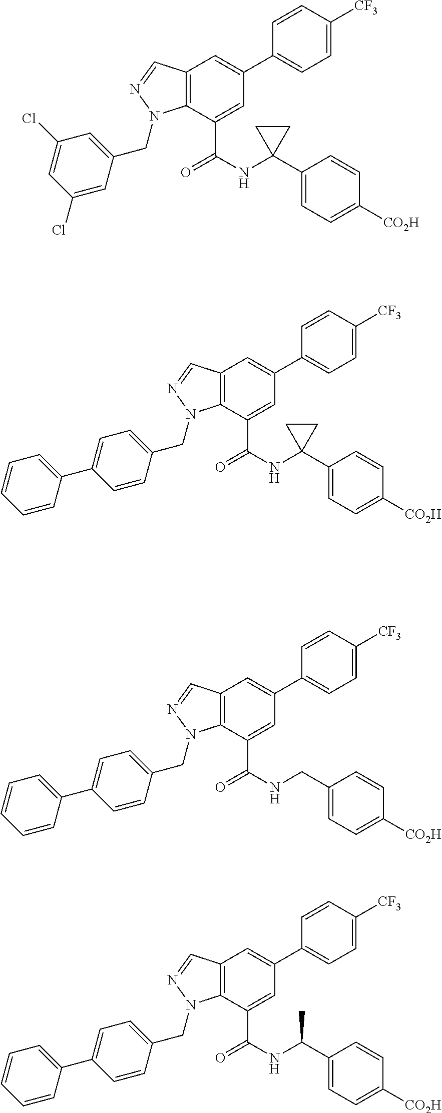 [SMILES: O=C(O)c1ccc(C2(NC(=O)c3cc(-c4ccc(C(F)(F)F)cc4)cc4cnn(Cc5cc(Cl)cc(Cl)c5)c34)CC2)cc1 ; O=C(O)c1ccc(C2(NC(=O)c3cc(-c4ccc(C(F)(F)F)cc4)cc4cnn(Cc5ccc(-c6ccccc6)cc5)c34)CC2)cc1 ; O=C(O)c1ccc(CNC(=O)c2cc(-c3ccc(C(F)(F)F)cc3)cc3cnn(Cc4ccc(-c5ccccc5)cc4)c23)cc1 ; C[C@H](NC(=O)c1cc(-c2ccc(C(F)(F)F)cc2)cc2cnn(Cc3ccc(-c4ccccc4)cc3)c12)c1ccc(C(=O)O)cc1]

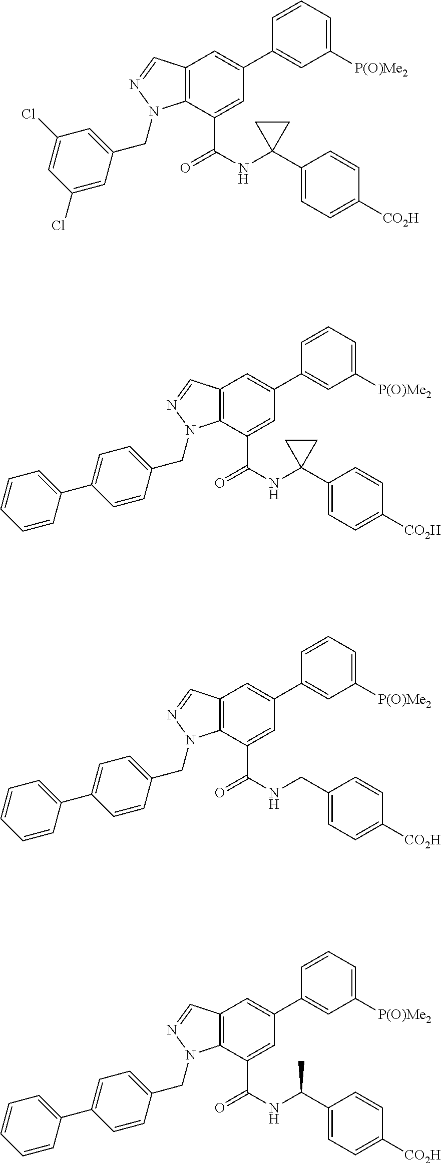

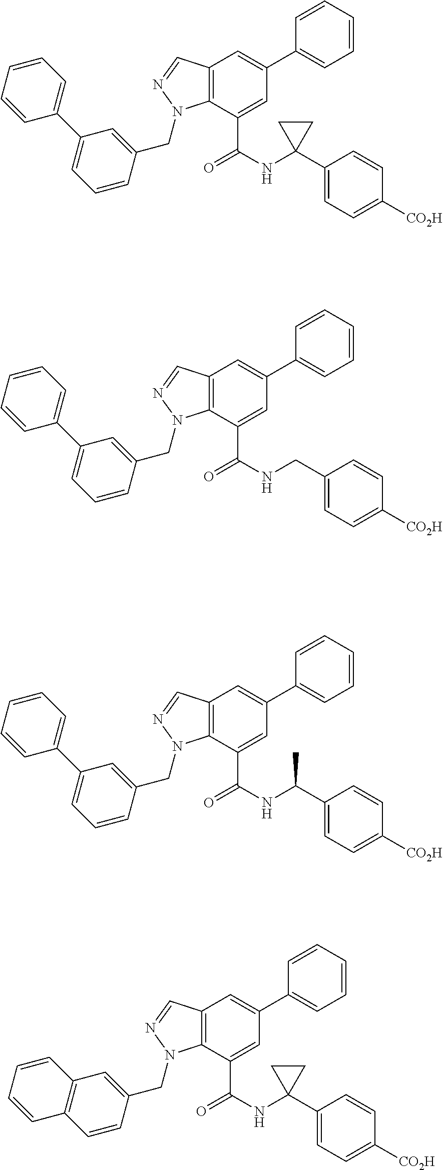 [SMILES: O=C(O)c1ccc(C2(NC(=O)c3cc(-c4ccccc4)cc4cnn(Cc5cccc(-c6ccccc6)c5)c34)CC2)cc1 ; O=C(O)c1ccc(CNC(=O)c2cc(-c3ccccc3)cc3cnn(Cc4cccc(-c5ccccc5)c4)c23)cc1 ; C[C@H](NC(=O)c1cc(-c2ccccc2)cc2cnn(Cc3cccc(-c4ccccc4)c3)c12)c1ccc(C(=O)O)cc1 ; O=C(O)c1ccc(C2(NC(=O)c3cc(-c4ccccc4)cc4cnn(Cc5ccc6ccccc6c5)c34)CC2)cc1]

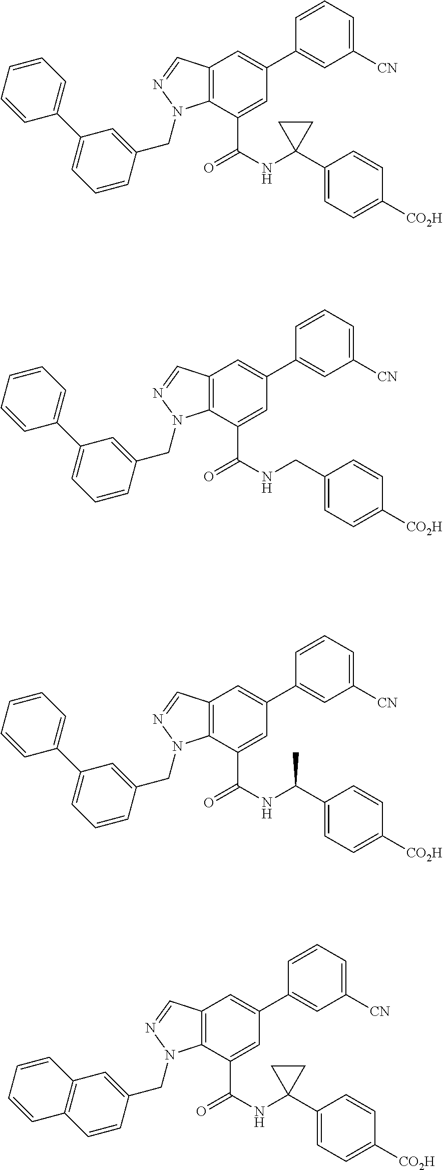 [SMILES: N#Cc1cccc(-c2cc(C(=O)NC3(c4ccc(C(=O)O)cc4)CC3)c3c(cnn3Cc3cccc(-c4ccccc4)c3)c2)c1 ; N#Cc1cccc(-c2cc(C(=O)NCc3ccc(C(=O)O)cc3)c3c(cnn3Cc3cccc(-c4ccccc4)c3)c2)c1 ; C[C@H](NC(=O)c1cc(-c2cccc(C#N)c2)cc2cnn(Cc3cccc(-c4ccccc4)c3)c12)c1ccc(C(=O)O)cc1 ; N#Cc1cccc(-c2cc(C(=O)NC3(c4ccc(C(=O)O)cc4)CC3)c3c(cnn3Cc3ccc4ccccc4c3)c2)c1]

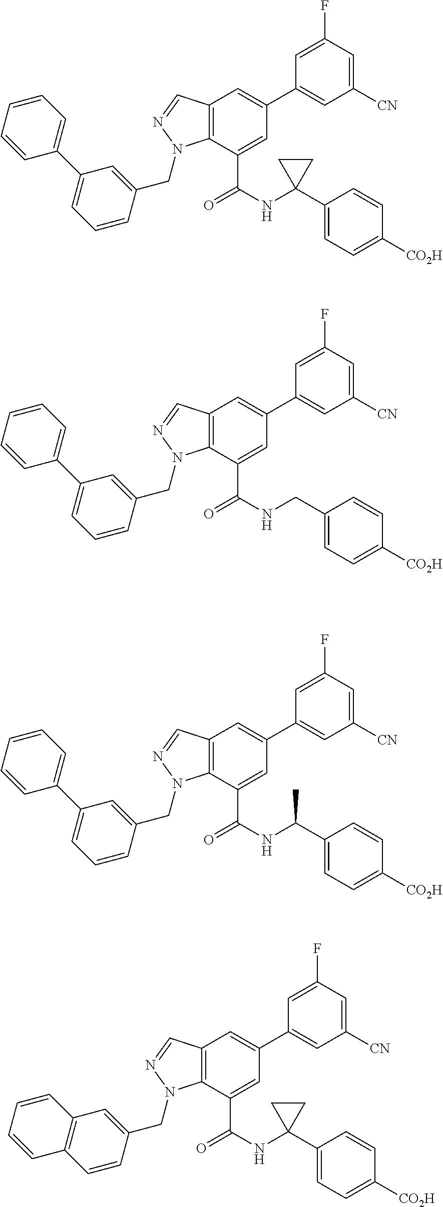 [SMILES: N#Cc1cc(F)cc(-c2cc(C(=O)NC3(c4ccc(C(=O)O)cc4)CC3)c3c(cnn3Cc3cccc(-c4ccccc4)c3)c2)c1 ; N#Cc1cc(F)cc(-c2cc(C(=O)NCc3ccc(C(=O)O)cc3)c3c(cnn3Cc3cccc(-c4ccccc4)c3)c2)c1 ; C[C@H](NC(=O)c1cc(-c2cc(F)cc(C#N)c2)cc2cnn(Cc3cccc(-c4ccccc4)c3)c12)c1ccc(C(=O)O)cc1 ; N#Cc1cc(F)cc(-c2cc(C(=O)NC3(c4ccc(C(=O)O)cc4)CC3)c3c(cnn3Cc3ccc4ccccc4c3)c2)c1]

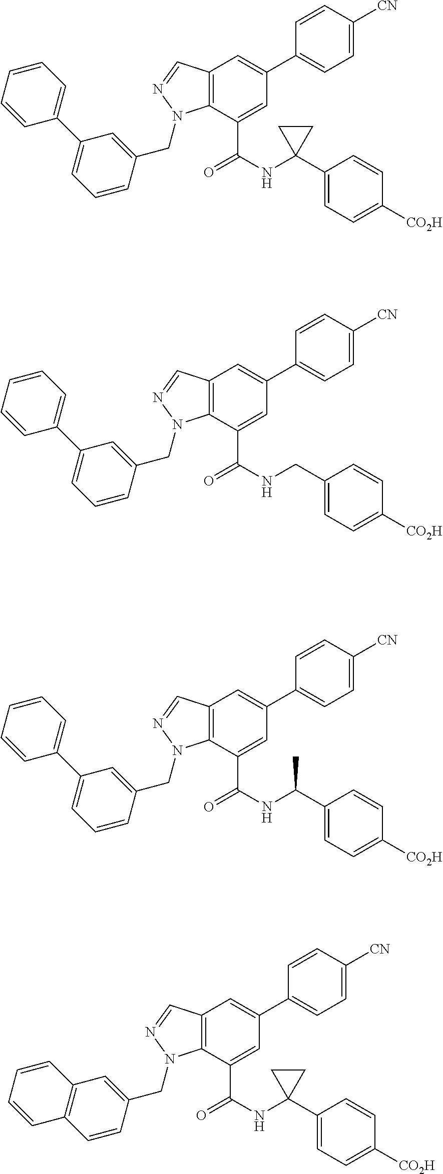 [SMILES: N#Cc1ccc(-c2cc(C(=O)NC3(c4ccc(C(=O)O)cc4)CC3)c3c(cnn3Cc3cccc(-c4ccccc4)c3)c2)cc1 ; N#Cc1ccc(-c2cc(C(=O)NCc3ccc(C(=O)O)cc3)c3c(cnn3Cc3cccc(-c4ccccc4)c3)c2)cc1 ; C[C@H](NC(=O)c1cc(-c2ccc(C#N)cc2)cc2cnn(Cc3cccc(-c4ccccc4)c3)c12)c1ccc(C(=O)O)cc1 ; N#Cc1ccc(-c2cc(C(=O)NC3(c4ccc(C(=O)O)cc4)CC3)c3c(cnn3Cc3ccc4ccccc4c3)c2)cc1]

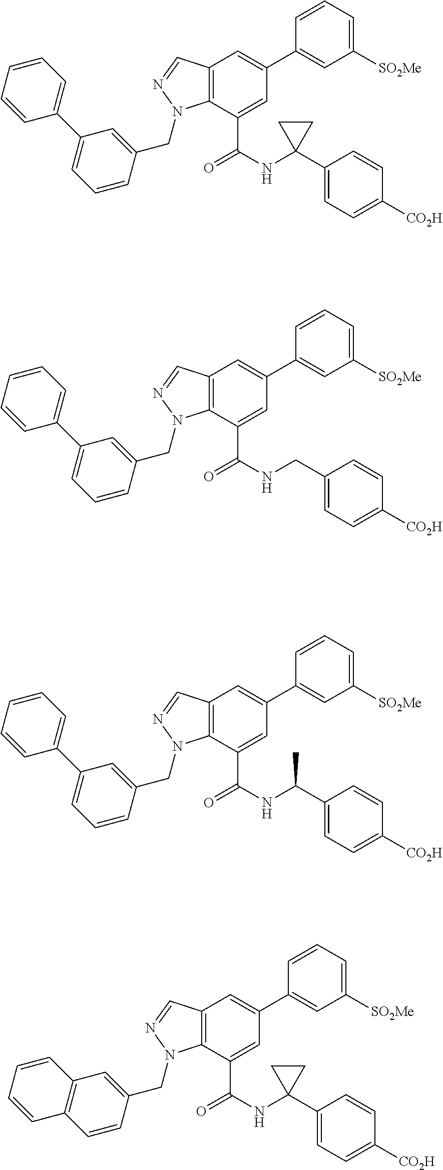 [SMILES: CS(=O)(=O)c1cccc(-c2cc(C(=O)NC3(c4ccc(C(=O)O)cc4)CC3)c3c(cnn3Cc3cccc(-c4ccccc4)c3)c2)c1 ; CS(=O)(=O)c1cccc(-c2cc(C(=O)NCc3ccc(C(=O)O)cc3)c3c(cnn3Cc3cccc(-c4ccccc4)c3)c2)c1 ; C[C@H](NC(=O)c1cc(-c2cccc(S(C)(=O)=O)c2)cc2cnn(Cc3cccc(-c4ccccc4)c3)c12)c1ccc(C(=O)O)cc1 ; CS(=O)(=O)c1cccc(-c2cc(C(=O)NC3(c4ccc(C(=O)O)cc4)CC3)c3c(cnn3Cc3ccc4ccccc4c3)c2)c1]

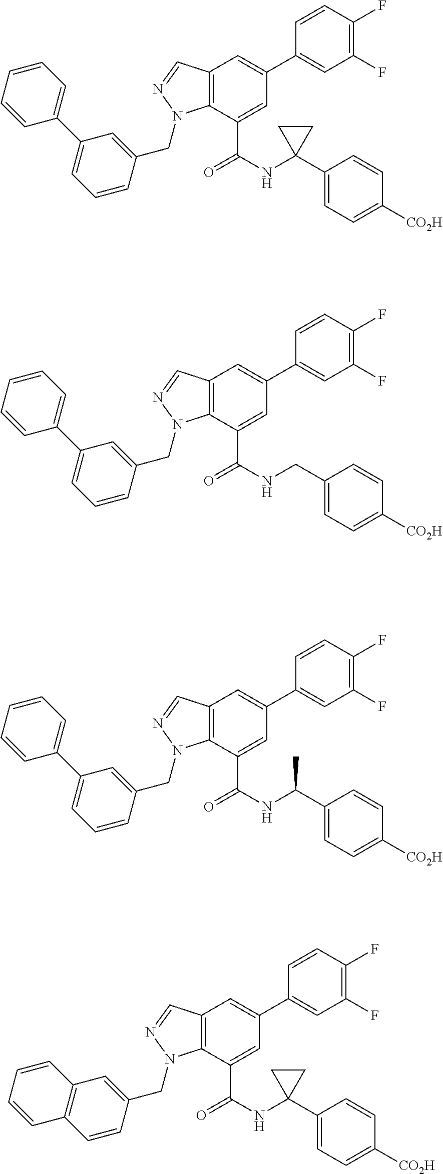 [SMILES: O=C(O)c1ccc(C2(NC(=O)c3cc(-c4ccc(F)c(F)c4)cc4cnn(Cc5cccc(-c6ccccc6)c5)c34)CC2)cc1 ; O=C(O)c1ccc(CNC(=O)c2cc(-c3ccc(F)c(F)c3)cc3cnn(Cc4cccc(-c5ccccc5)c4)c23)cc1 ; C[C@H](NC(=O)c1cc(-c2ccc(F)c(F)c2)cc2cnn(Cc3cccc(-c4ccccc4)c3)c12)c1ccc(C(=O)O)cc1 ; O=C(O)c1ccc(C2(NC(=O)c3cc(-c4ccc(F)c(F)c4)cc4cnn(Cc5ccc6ccccc6c5)c34)CC2)cc1]

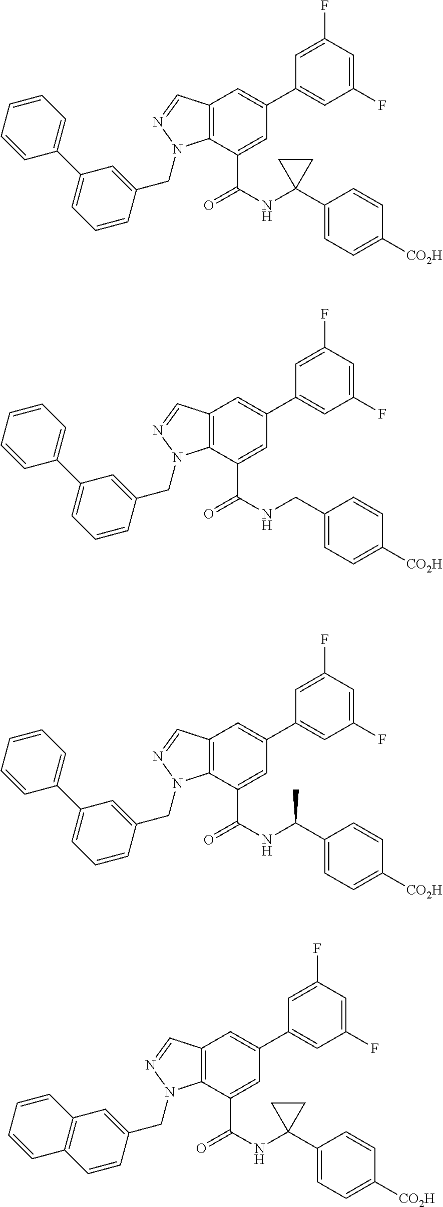 [SMILES: O=C(O)c1ccc(C2(NC(=O)c3cc(-c4cc(F)cc(F)c4)cc4cnn(Cc5cccc(-c6ccccc6)c5)c34)CC2)cc1 ; O=C(O)c1ccc(CNC(=O)c2cc(-c3cc(F)cc(F)c3)cc3cnn(Cc4cccc(-c5ccccc5)c4)c23)cc1 ; C[C@H](NC(=O)c1cc(-c2cc(F)cc(F)c2)cc2cnn(Cc3cccc(-c4ccccc4)c3)c12)c1ccc(C(=O)O)cc1 ; O=C(O)c1ccc(C2(NC(=O)c3cc(-c4cc(F)cc(F)c4)cc4cnn(Cc5ccc6ccccc6c5)c34)CC2)cc1]

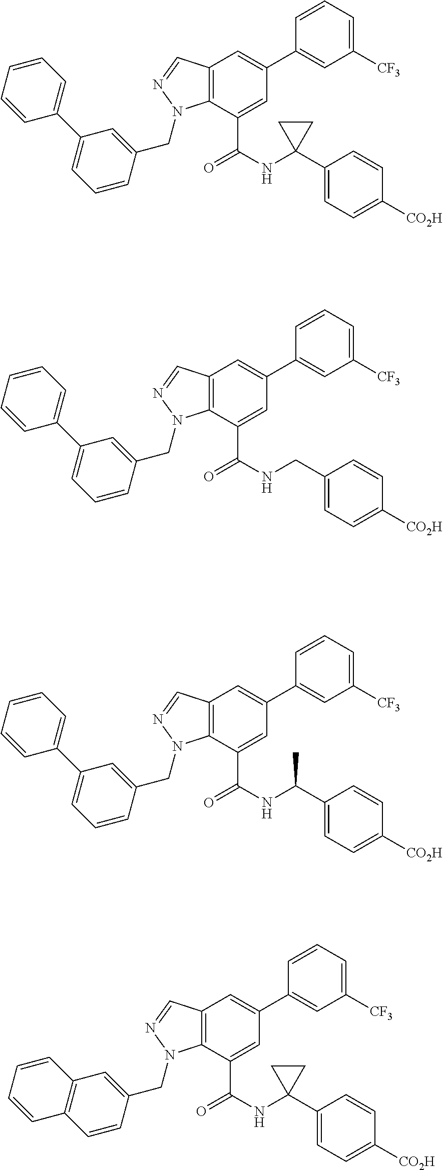 [SMILES: O=C(O)c1ccc(C2(NC(=O)c3cc(-c4cccc(C(F)(F)F)c4)cc4cnn(Cc5cccc(-c6ccccc6)c5)c34)CC2)cc1 ; O=C(O)c1ccc(CNC(=O)c2cc(-c3cccc(C(F)(F)F)c3)cc3cnn(Cc4cccc(-c5ccccc5)c4)c23)cc1 ; C[C@H](NC(=O)c1cc(-c2cccc(C(F)(F)F)c2)cc2cnn(Cc3cccc(-c4ccccc4)c3)c12)c1ccc(C(=O)O)cc1 ; O=C(O)c1ccc(C2(NC(=O)c3cc(-c4cccc(C(F)(F)F)c4)cc4cnn(Cc5ccc6ccccc6c5)c34)CC2)cc1]

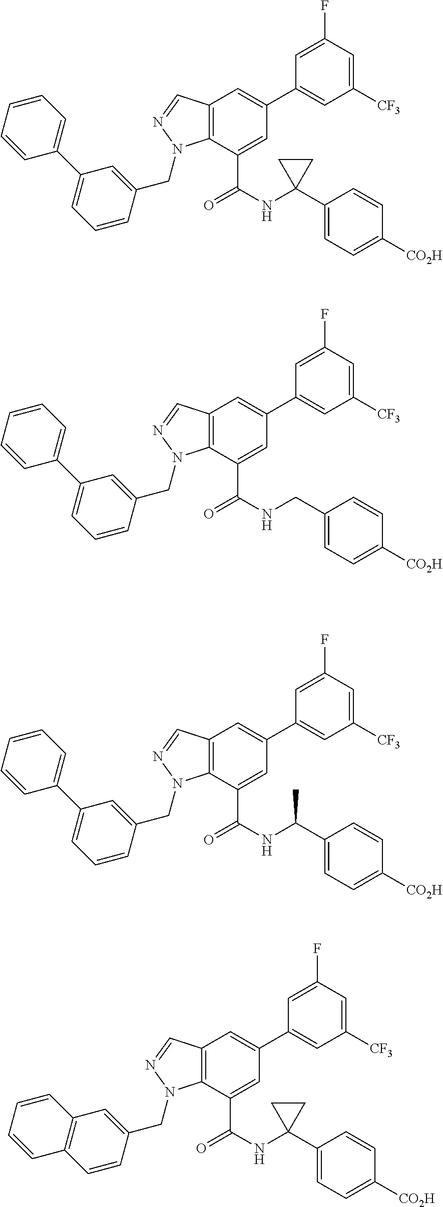 [SMILES: O=C(O)c1ccc(C2(NC(=O)c3cc(-c4cc(F)cc(C(F)(F)F)c4)cc4cnn(Cc5cccc(-c6ccccc6)c5)c34)CC2)cc1 ; O=C(O)c1ccc(CNC(=O)c2cc(-c3cc(F)cc(C(F)(F)F)c3)cc3cnn(Cc4cccc(-c5ccccc5)c4)c23)cc1 ; C[C@H](NC(=O)c1cc(-c2cc(F)cc(C(F)(F)F)c2)cc2cnn(Cc3cccc(-c4ccccc4)c3)c12)c1ccc(C(=O)O)cc1 ; O=C(O)c1ccc(C2(NC(=O)c3cc(-c4cc(F)cc(C(F)(F)F)c4)cc4cnn(Cc5ccc6ccccc6c5)c34)CC2)cc1]

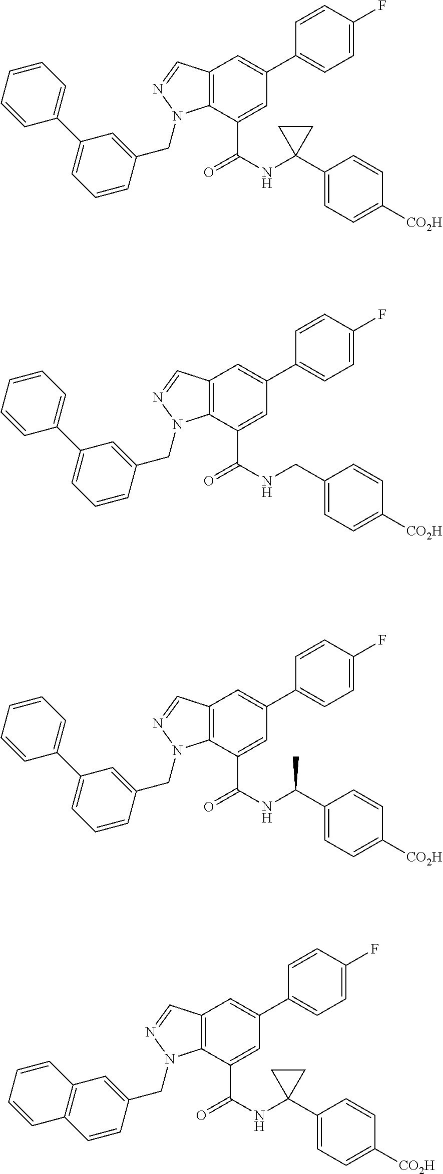 [SMILES: O=C(O)c1ccc(C2(NC(=O)c3cc(-c4ccc(F)cc4)cc4cnn(Cc5cccc(-c6ccccc6)c5)c34)CC2)cc1 ; O=C(O)c1ccc(CNC(=O)c2cc(-c3ccc(F)cc3)cc3cnn(Cc4cccc(-c5ccccc5)c4)c23)cc1 ; C[C@H](NC(=O)c1cc(-c2ccc(F)cc2)cc2cnn(Cc3cccc(-c4ccccc4)c3)c12)c1ccc(C(=O)O)cc1 ; O=C(O)c1ccc(C2(NC(=O)c3cc(-c4ccc(F)cc4)cc4cnn(Cc5ccc6ccccc6c5)c34)CC2)cc1]

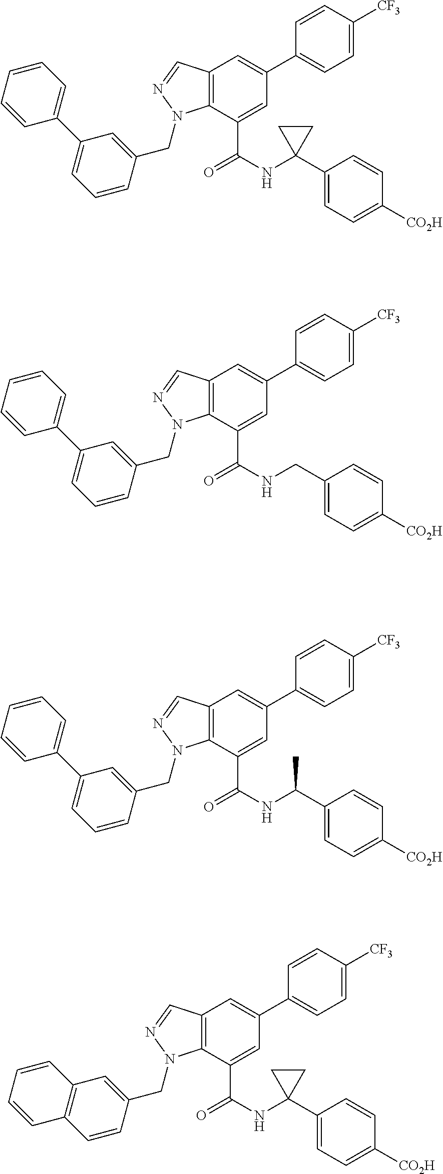 [SMILES: O=C(O)c1ccc(C2(NC(=O)c3cc(-c4ccc(C(F)(F)F)cc4)cc4cnn(Cc5cccc(-c6ccccc6)c5)c34)CC2)cc1 ; O=C(O)c1ccc(CNC(=O)c2cc(-c3ccc(C(F)(F)F)cc3)cc3cnn(Cc4cccc(-c5ccccc5)c4)c23)cc1 ; C[C@H](NC(=O)c1cc(-c2ccc(C(F)(F)F)cc2)cc2cnn(Cc3cccc(-c4ccccc4)c3)c12)c1ccc(C(=O)O)cc1 ; O=C(O)c1ccc(C2(NC(=O)c3cc(-c4ccc(C(F)(F)F)cc4)cc4cnn(Cc5ccc6ccccc6c5)c34)CC2)cc1]

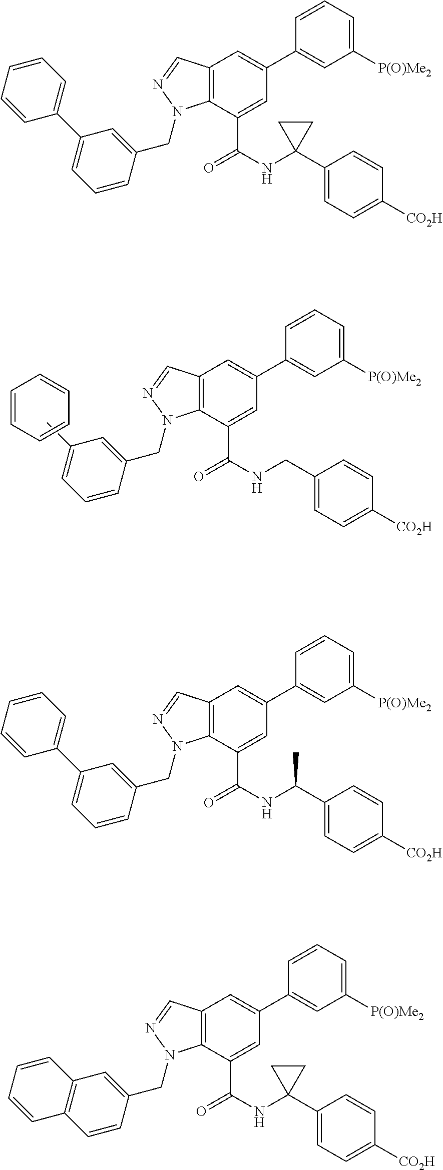

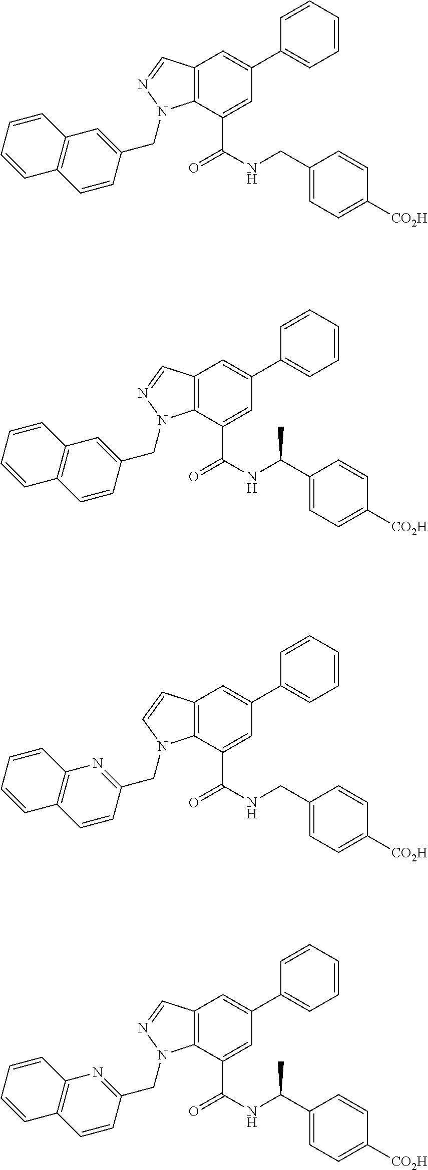 [SMILES: O=C(O)c1ccc(CNC(=O)c2cc(-c3ccccc3)cc3cnn(Cc4ccc5ccccc5c4)c23)cc1 ; C[C@H](NC(=O)c1cc(-c2ccccc2)cc2cnn(Cc3ccc4ccccc4c3)c12)c1ccc(C(=O)O)cc1 ; O=C(O)c1ccc(CNC(=O)c2cc(-c3ccccc3)cc3ccn(Cc4ccc5ccccc5n4)c23)cc1 ; C[C@H](NC(=O)c1cc(-c2ccccc2)cc2cnn(Cc3ccc4ccccc4n3)c12)c1ccc(C(=O)O)cc1]

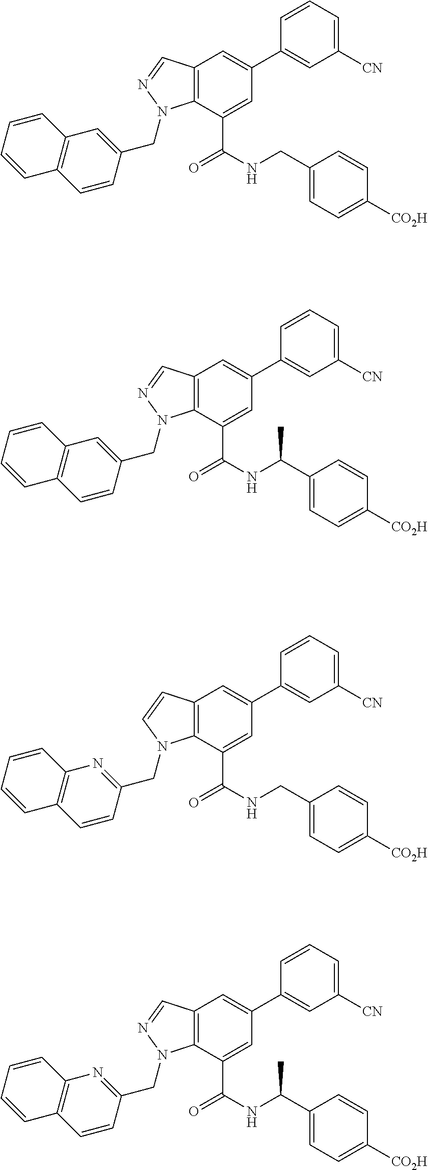 [SMILES: N#Cc1cccc(-c2cc(C(=O)NCc3ccc(C(=O)O)cc3)c3c(cnn3Cc3ccc4ccccc4c3)c2)c1 ; C[C@H](NC(=O)c1cc(-c2cccc(C#N)c2)cc2cnn(Cc3ccc4ccccc4c3)c12)c1ccc(C(=O)O)cc1 ; N#Cc1cccc(-c2cc(C(=O)NCc3ccc(C(=O)O)cc3)c3c(ccn3Cc3ccc4ccccc4n3)c2)c1 ; C[C@H](NC(=O)c1cc(-c2cccc(C#N)c2)cc2cnn(Cc3ccc4ccccc4n3)c12)c1ccc(C(=O)O)cc1]

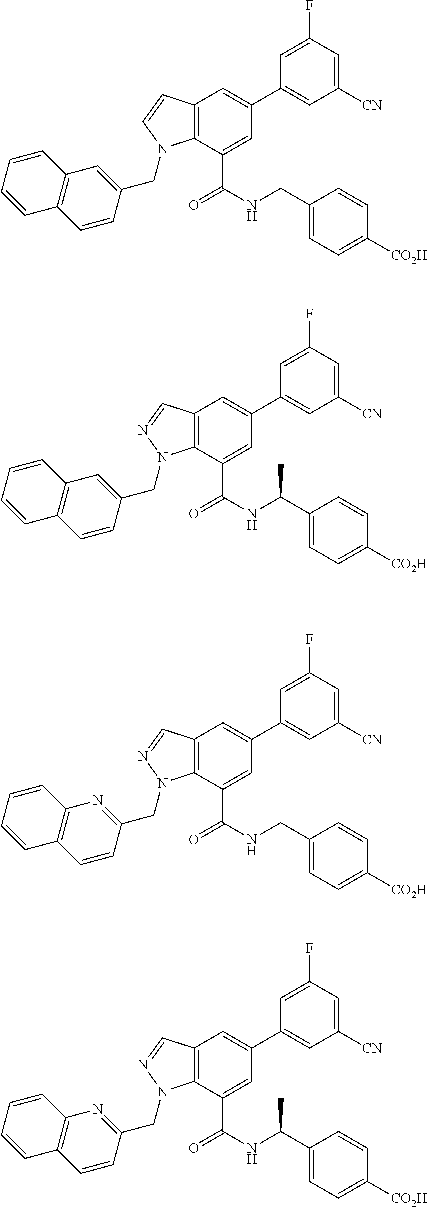 [SMILES: N#Cc1cc(F)cc(-c2cc(C(=O)NCc3ccc(C(=O)O)cc3)c3c(ccn3Cc3ccc4ccccc4c3)c2)c1 ; C[C@H](NC(=O)c1cc(-c2cc(F)cc(C#N)c2)cc2cnn(Cc3ccc4ccccc4c3)c12)c1ccc(C(=O)O)cc1 ; N#Cc1cc(F)cc(-c2cc(C(=O)NCc3ccc(C(=O)O)cc3)c3c(cnn3Cc3ccc4ccccc4n3)c2)c1 ; C[C@H](NC(=O)c1cc(-c2cc(F)cc(C#N)c2)cc2cnn(Cc3ccc4ccccc4n3)c12)c1ccc(C(=O)O)cc1]

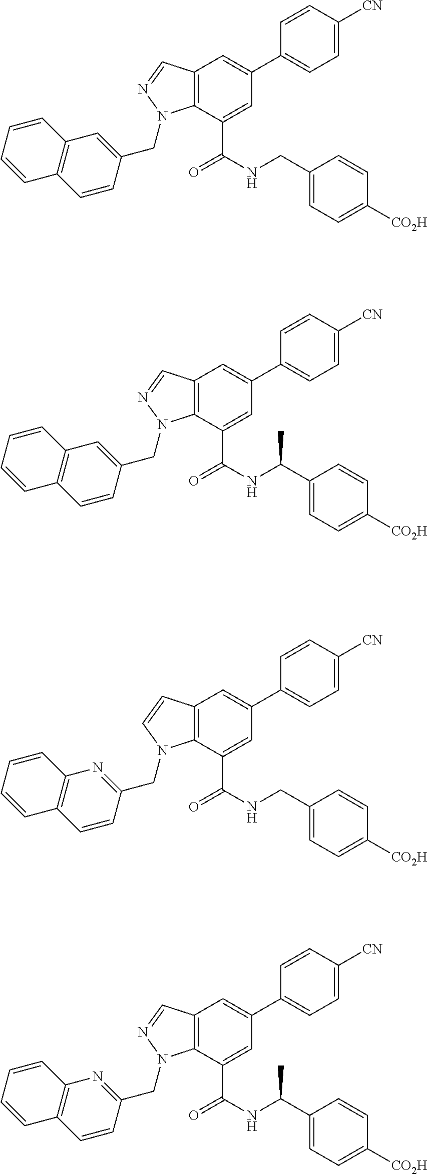 [SMILES: N#Cc1ccc(-c2cc(C(=O)NCc3ccc(C(=O)O)cc3)c3c(cnn3Cc3ccc4ccccc4c3)c2)cc1 ; C[C@H](NC(=O)c1cc(-c2ccc(C#N)cc2)cc2cnn(Cc3ccc4ccccc4c3)c12)c1ccc(C(=O)O)cc1 ; N#Cc1ccc(-c2cc(C(=O)NCc3ccc(C(=O)O)cc3)c3c(ccn3Cc3ccc4ccccc4n3)c2)cc1 ; C[C@H](NC(=O)c1cc(-c2ccc(C#N)cc2)cc2cnn(Cc3ccc4ccccc4n3)c12)c1ccc(C(=O)O)cc1]

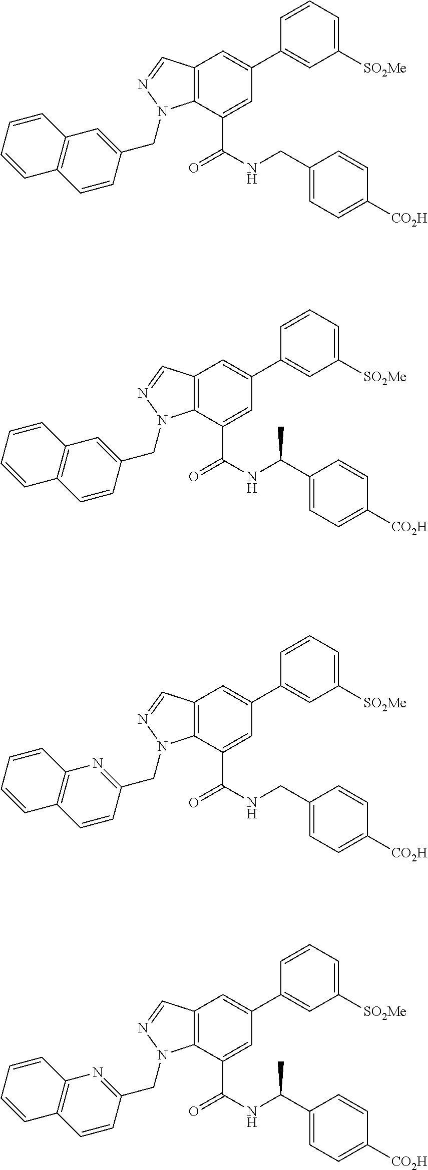 [SMILES: CS(=O)(=O)c1cccc(-c2cc(C(=O)NCc3ccc(C(=O)O)cc3)c3c(cnn3Cc3ccc4ccccc4c3)c2)c1 ; C[C@H](NC(=O)c1cc(-c2cccc(S(C)(=O)=O)c2)cc2cnn(Cc3ccc4ccccc4c3)c12)c1ccc(C(=O)O)cc1 ; CS(=O)(=O)c1cccc(-c2cc(C(=O)NCc3ccc(C(=O)O)cc3)c3c(cnn3Cc3ccc4ccccc4n3)c2)c1 ; C[C@H](NC(=O)c1cc(-c2cccc(S(C)(=O)=O)c2)cc2cnn(Cc3ccc4ccccc4n3)c12)c1ccc(C(=O)O)cc1]

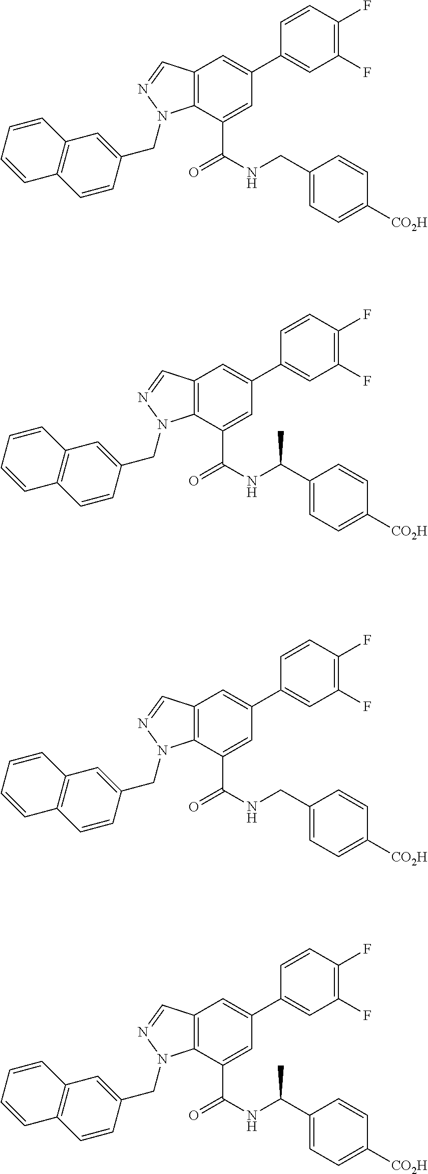 [SMILES: O=C(O)c1ccc(CNC(=O)c2cc(-c3ccc(F)c(F)c3)cc3cnn(Cc4ccc5ccccc5c4)c23)cc1 ; C[C@H](NC(=O)c1cc(-c2ccc(F)c(F)c2)cc2cnn(Cc3ccc4ccccc4c3)c12)c1ccc(C(=O)O)cc1 ; O=C(O)c1ccc(CNC(=O)c2cc(-c3ccc(F)c(F)c3)cc3cnn(Cc4ccc5ccccc5c4)c23)cc1 ; C[C@H](NC(=O)c1cc(-c2ccc(F)c(F)c2)cc2cnn(Cc3ccc4ccccc4c3)c12)c1ccc(C(=O)O)cc1]

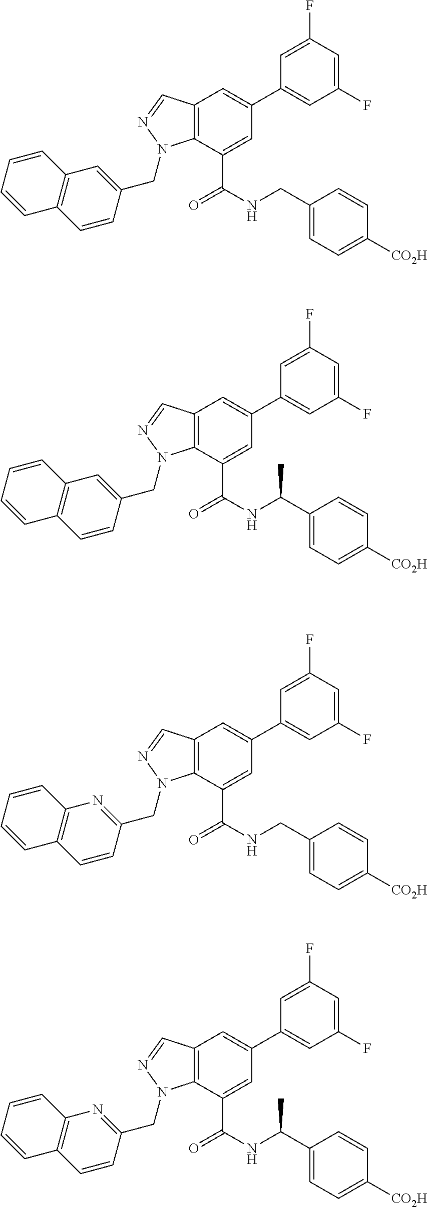 [SMILES: O=C(O)c1ccc(CNC(=O)c2cc(-c3cc(F)cc(F)c3)cc3cnn(Cc4ccc5ccccc5c4)c23)cc1 ; C[C@H](NC(=O)c1cc(-c2cc(F)cc(F)c2)cc2cnn(Cc3ccc4ccccc4c3)c12)c1ccc(C(=O)O)cc1 ; O=C(O)c1ccc(CNC(=O)c2cc(-c3cc(F)cc(F)c3)cc3cnn(Cc4ccc5ccccc5n4)c23)cc1 ; C[C@H](NC(=O)c1cc(-c2cc(F)cc(F)c2)cc2cnn(Cc3ccc4ccccc4n3)c12)c1ccc(C(=O)O)cc1]

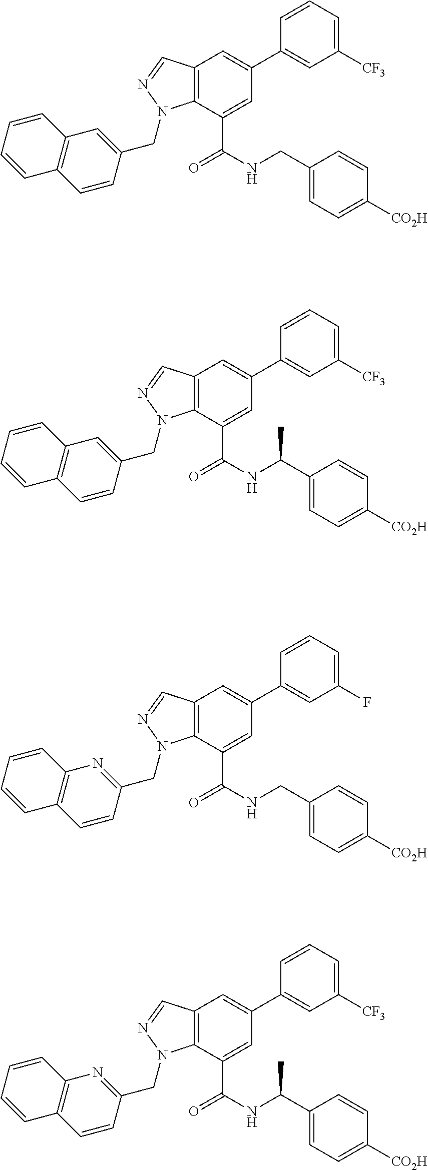 [SMILES: O=C(O)c1ccc(CNC(=O)c2cc(-c3cccc(C(F)(F)F)c3)cc3cnn(Cc4ccc5ccccc5c4)c23)cc1 ; C[C@H](NC(=O)c1cc(-c2cccc(C(F)(F)F)c2)cc2cnn(Cc3ccc4ccccc4c3)c12)c1ccc(C(=O)O)cc1 ; O=C(O)c1ccc(CNC(=O)c2cc(-c3cccc(F)c3)cc3cnn(Cc4ccc5ccccc5n4)c23)cc1 ; C[C@H](NC(=O)c1cc(-c2cccc(C(F)(F)F)c2)cc2cnn(Cc3ccc4ccccc4n3)c12)c1ccc(C(=O)O)cc1]

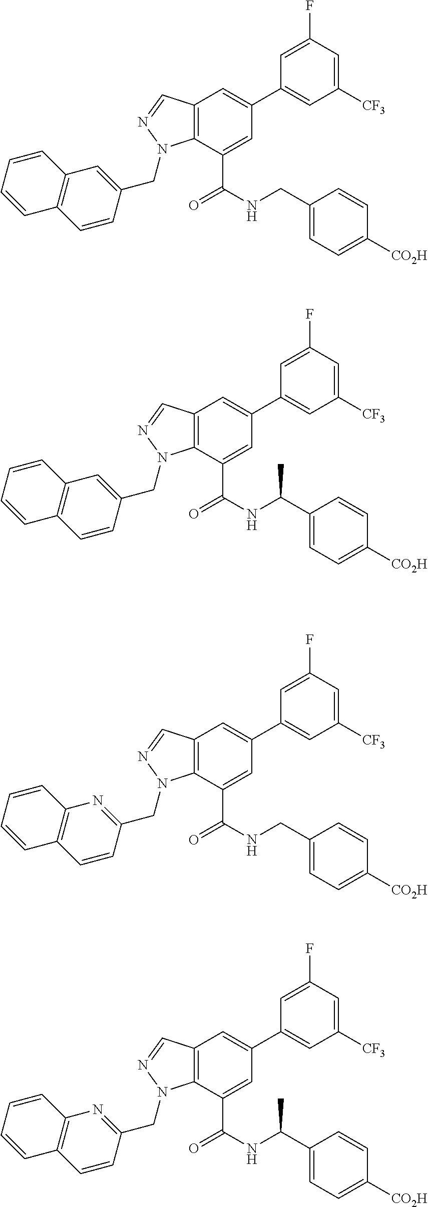 [SMILES: O=C(O)c1ccc(CNC(=O)c2cc(-c3cc(F)cc(C(F)(F)F)c3)cc3cnn(Cc4ccc5ccccc5c4)c23)cc1 ; C[C@H](NC(=O)c1cc(-c2cc(F)cc(C(F)(F)F)c2)cc2cnn(Cc3ccc4ccccc4c3)c12)c1ccc(C(=O)O)cc1 ; O=C(O)c1ccc(CNC(=O)c2cc(-c3cc(F)cc(C(F)(F)F)c3)cc3cnn(Cc4ccc5ccccc5n4)c23)cc1 ; C[C@H](NC(=O)c1cc(-c2cc(F)cc(C(F)(F)F)c2)cc2cnn(Cc3ccc4ccccc4n3)c12)c1ccc(C(=O)O)cc1]

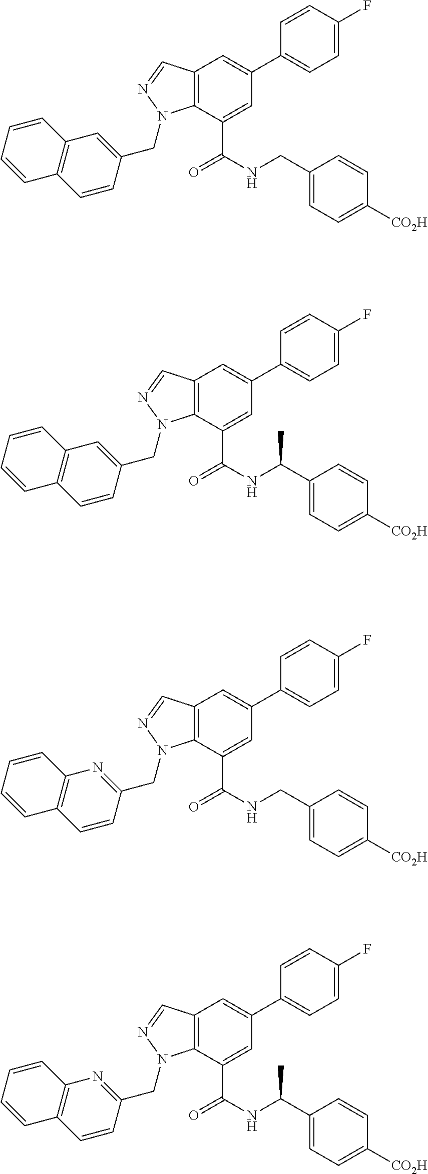 [SMILES: O=C(O)c1ccc(CNC(=O)c2cc(-c3ccc(F)cc3)cc3cnn(Cc4ccc5ccccc5c4)c23)cc1 ; C[C@H](NC(=O)c1cc(-c2ccc(F)cc2)cc2cnn(Cc3ccc4ccccc4c3)c12)c1ccc(C(=O)O)cc1 ; O=C(O)c1ccc(CNC(=O)c2cc(-c3ccc(F)cc3)cc3cnn(Cc4ccc5ccccc5n4)c23)cc1 ; C[C@H](NC(=O)c1cc(-c2ccc(F)cc2)cc2cnn(Cc3ccc4ccccc4n3)c12)c1ccc(C(=O)O)cc1]

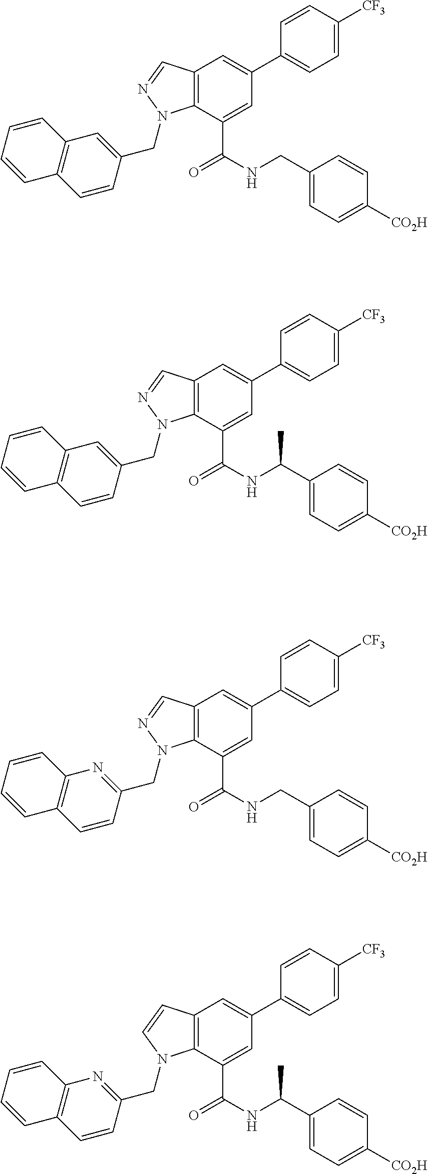 [SMILES: O=C(O)c1ccc(CNC(=O)c2cc(-c3ccc(C(F)(F)F)cc3)cc3cnn(Cc4ccc5ccccc5c4)c23)cc1 ; C[C@H](NC(=O)c1cc(-c2ccc(C(F)(F)F)cc2)cc2cnn(Cc3ccc4ccccc4c3)c12)c1ccc(C(=O)O)cc1 ; O=C(O)c1ccc(CNC(=O)c2cc(-c3ccc(C(F)(F)F)cc3)cc3cnn(Cc4ccc5ccccc5n4)c23)cc1 ; C[C@H](NC(=O)c1cc(-c2ccc(C(F)(F)F)cc2)cc2ccn(Cc3ccc4ccccc4n3)c12)c1ccc(C(=O)O)cc1]

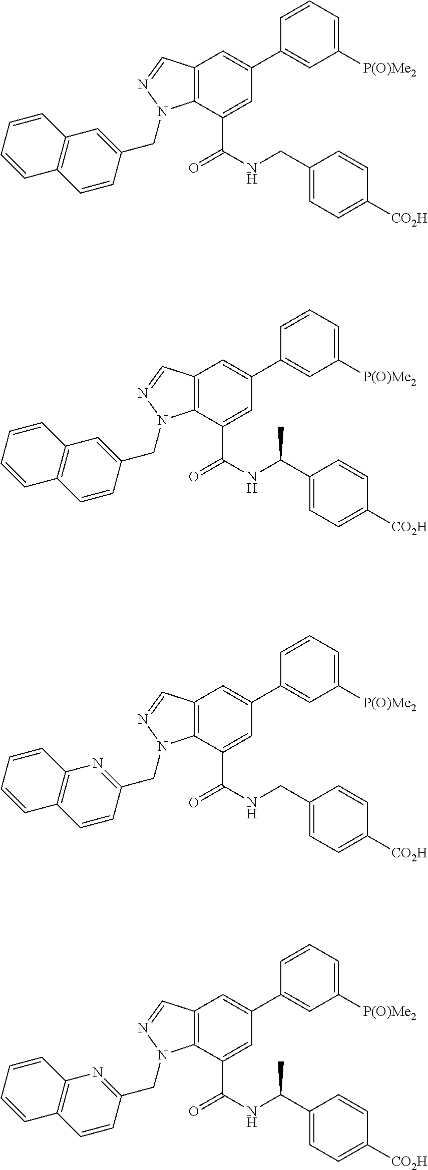

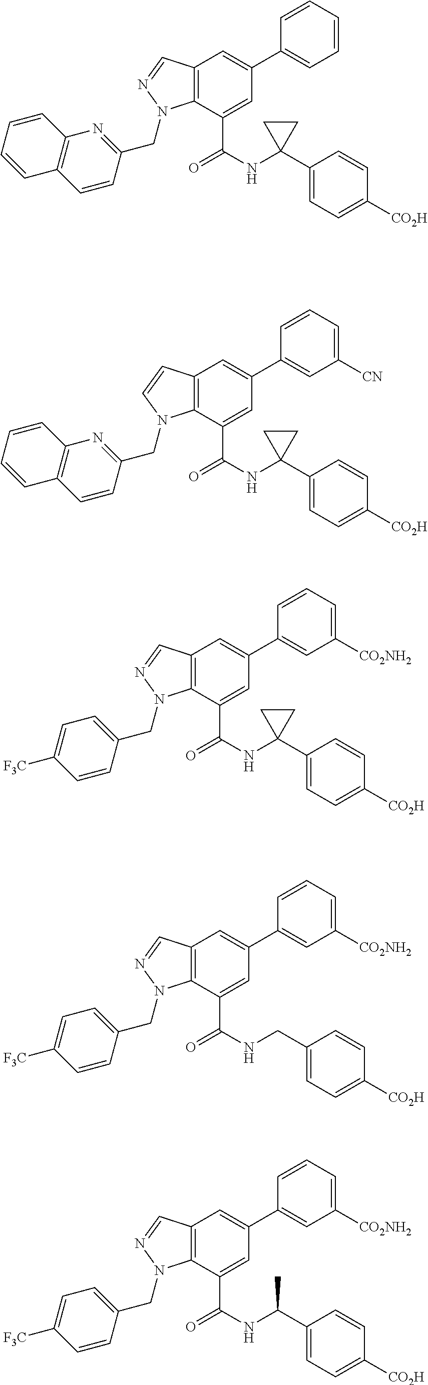

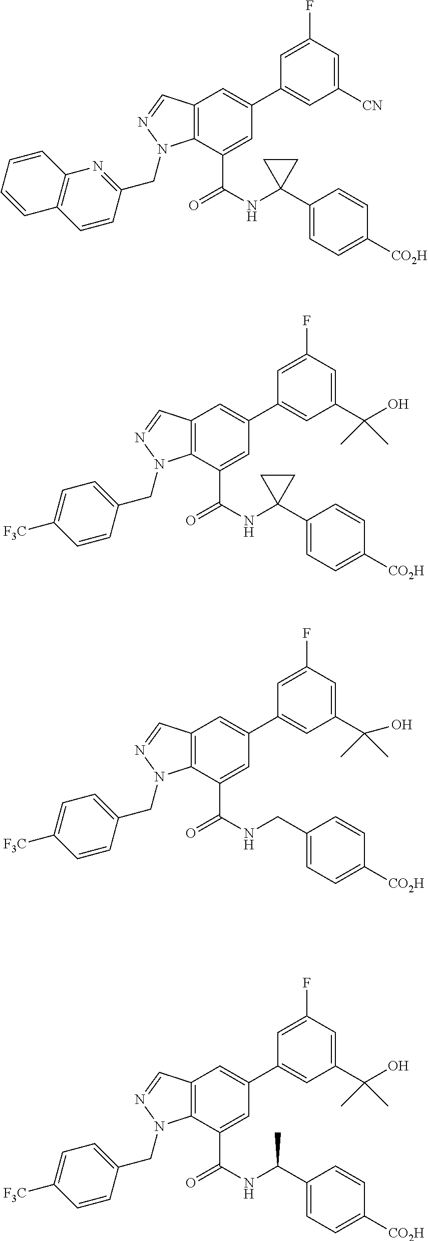 [SMILES: N#Cc1cc(F)cc(-c2cc(C(=O)NC3(c4ccc(C(=O)O)cc4)CC3)c3c(cnn3Cc3ccc4ccccc4n3)c2)c1 ; CC(C)(O)c1cc(F)cc(-c2cc(C(=O)NC3(c4ccc(C(=O)O)cc4)CC3)c3c(cnn3Cc3ccc(C(F)(F)F)cc3)c2)c1 ; CC(C)(O)c1cc(F)cc(-c2cc(C(=O)NCc3ccc(C(=O)O)cc3)c3c(cnn3Cc3ccc(C(F)(F)F)cc3)c2)c1 ; C[C@H](NC(=O)c1cc(-c2cc(F)cc(C(C)(C)O)c2)cc2cnn(Cc3ccc(C(F)(F)F)cc3)c12)c1ccc(C(=O)O)cc1]

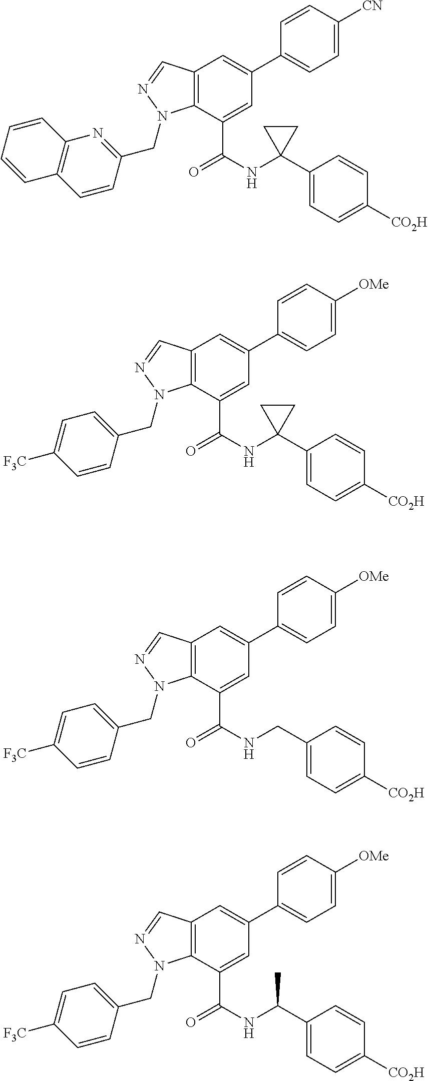 [SMILES: N#Cc1ccc(-c2cc(C(=O)NC3(c4ccc(C(=O)O)cc4)CC3)c3c(cnn3Cc3ccc4ccccc4n3)c2)cc1 ; COc1ccc(-c2cc(C(=O)NC3(c4ccc(C(=O)O)cc4)CC3)c3c(cnn3Cc3ccc(C(F)(F)F)cc3)c2)cc1 ; COc1ccc(-c2cc(C(=O)NCc3ccc(C(=O)O)cc3)c3c(cnn3Cc3ccc(C(F)(F)F)cc3)c2)cc1 ; COc1ccc(-c2cc(C(=O)N[C@@H](C)c3ccc(C(=O)O)cc3)c3c(cnn3Cc3ccc(C(F)(F)F)cc3)c2)cc1]

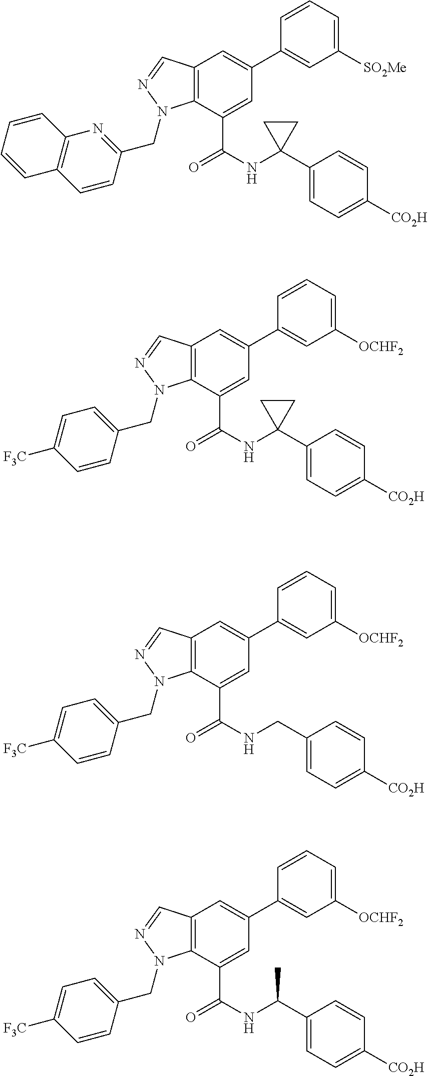 [SMILES: CS(=O)(=O)c1cccc(-c2cc(C(=O)NC3(c4ccc(C(=O)O)cc4)CC3)c3c(cnn3Cc3ccc4ccccc4n3)c2)c1 ; O=C(O)c1ccc(C2(NC(=O)c3cc(-c4cccc(OC(F)F)c4)cc4cnn(Cc5ccc(C(F)(F)F)cc5)c34)CC2)cc1 ; O=C(O)c1ccc(CNC(=O)c2cc(-c3cccc(OC(F)F)c3)cc3cnn(Cc4ccc(C(F)(F)F)cc4)c23)cc1 ; C[C@H](NC(=O)c1cc(-c2cccc(OC(F)F)c2)cc2cnn(Cc3ccc(C(F)(F)F)cc3)c12)c1ccc(C(=O)O)cc1]

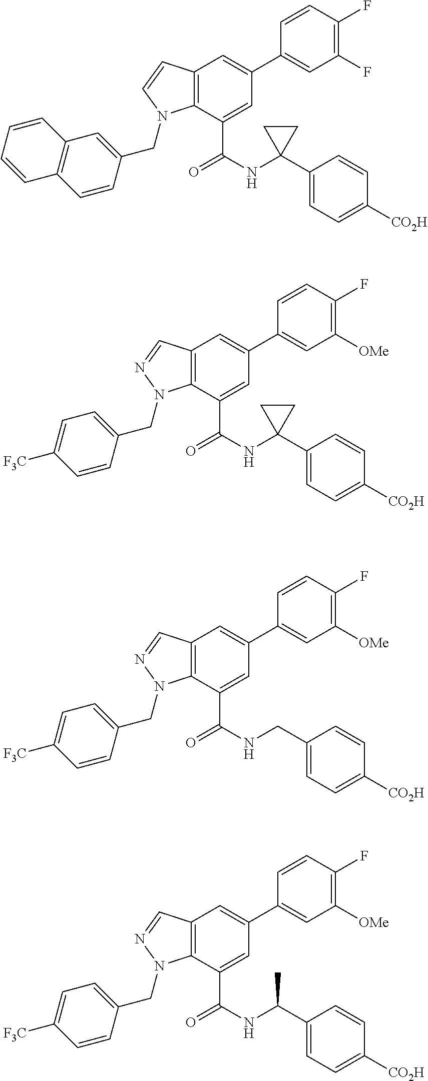 [SMILES: O=C(O)c1ccc(C2(NC(=O)c3cc(-c4ccc(F)c(F)c4)cc4ccn(Cc5ccc6ccccc6c5)c34)CC2)cc1 ; COc1cc(-c2cc(C(=O)NC3(c4ccc(C(=O)O)cc4)CC3)c3c(cnn3Cc3ccc(C(F)(F)F)cc3)c2)ccc1F ; COc1cc(-c2cc(C(=O)NCc3ccc(C(=O)O)cc3)c3c(cnn3Cc3ccc(C(F)(F)F)cc3)c2)ccc1F ; COc1cc(-c2cc(C(=O)N[C@@H](C)c3ccc(C(=O)O)cc3)c3c(cnn3Cc3ccc(C(F)(F)F)cc3)c2)ccc1F]

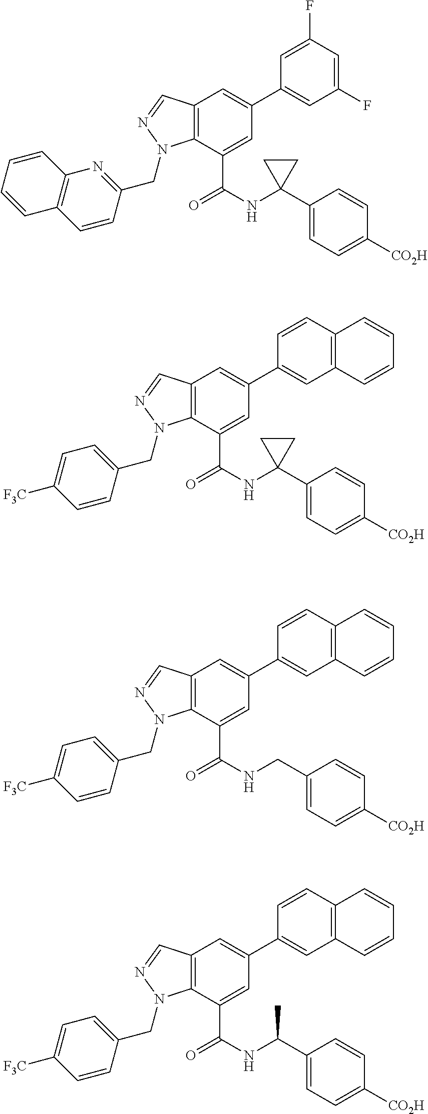 [SMILES: O=C(O)c1ccc(C2(NC(=O)c3cc(-c4cc(F)cc(F)c4)cc4cnn(Cc5ccc6ccccc6n5)c34)CC2)cc1 ; O=C(O)c1ccc(C2(NC(=O)c3cc(-c4ccc5ccccc5c4)cc4cnn(Cc5ccc(C(F)(F)F)cc5)c34)CC2)cc1 ; O=C(O)c1ccc(CNC(=O)c2cc(-c3ccc4ccccc4c3)cc3cnn(Cc4ccc(C(F)(F)F)cc4)c23)cc1 ; C[C@H](NC(=O)c1cc(-c2ccc3ccccc3c2)cc2cnn(Cc3ccc(C(F)(F)F)cc3)c12)c1ccc(C(=O)O)cc1]

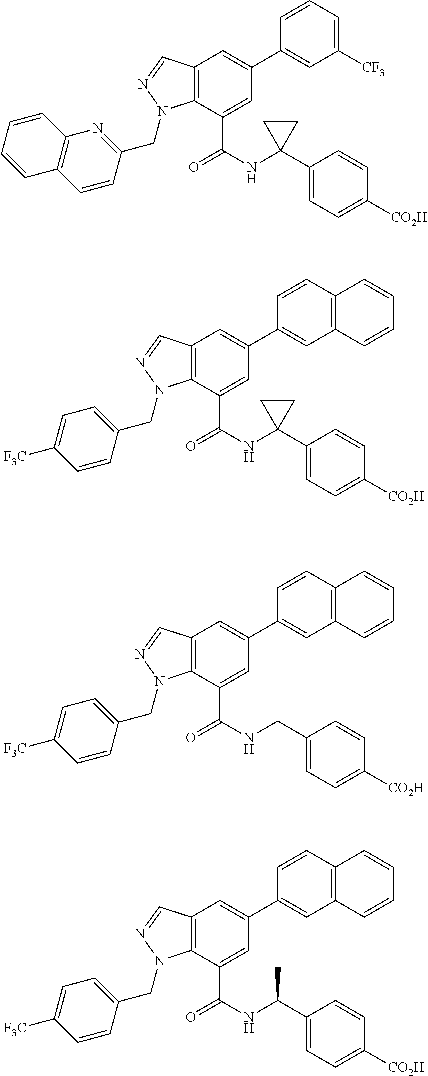 [SMILES: O=C(O)c1ccc(C2(NC(=O)c3cc(-c4cccc(C(F)(F)F)c4)cc4cnn(Cc5ccc6ccccc6n5)c34)CC2)cc1 ; O=C(O)c1ccc(C2(NC(=O)c3cc(-c4ccc5ccccc5c4)cc4cnn(Cc5ccc(C(F)(F)F)cc5)c34)CC2)cc1 ; O=C(O)c1ccc(CNC(=O)c2cc(-c3ccc4ccccc4c3)cc3cnn(Cc4ccc(C(F)(F)F)cc4)c23)cc1 ; C[C@H](NC(=O)c1cc(-c2ccc3ccccc3c2)cc2cnn(Cc3ccc(C(F)(F)F)cc3)c12)c1ccc(C(=O)O)cc1]

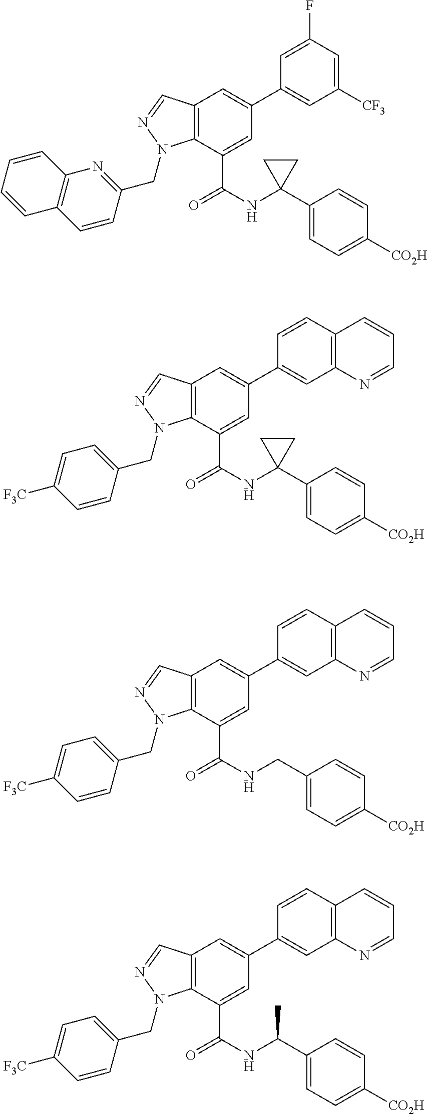 [SMILES: O=C(O)c1ccc(C2(NC(=O)c3cc(-c4cc(F)cc(C(F)(F)F)c4)cc4cnn(Cc5ccc6ccccc6n5)c34)CC2)cc1 ; O=C(O)c1ccc(C2(NC(=O)c3cc(-c4ccc5cccnc5c4)cc4cnn(Cc5ccc(C(F)(F)F)cc5)c34)CC2)cc1 ; O=C(O)c1ccc(CNC(=O)c2cc(-c3ccc4cccnc4c3)cc3cnn(Cc4ccc(C(F)(F)F)cc4)c23)cc1 ; C[C@H](NC(=O)c1cc(-c2ccc3cccnc3c2)cc2cnn(Cc3ccc(C(F)(F)F)cc3)c12)c1ccc(C(=O)O)cc1]

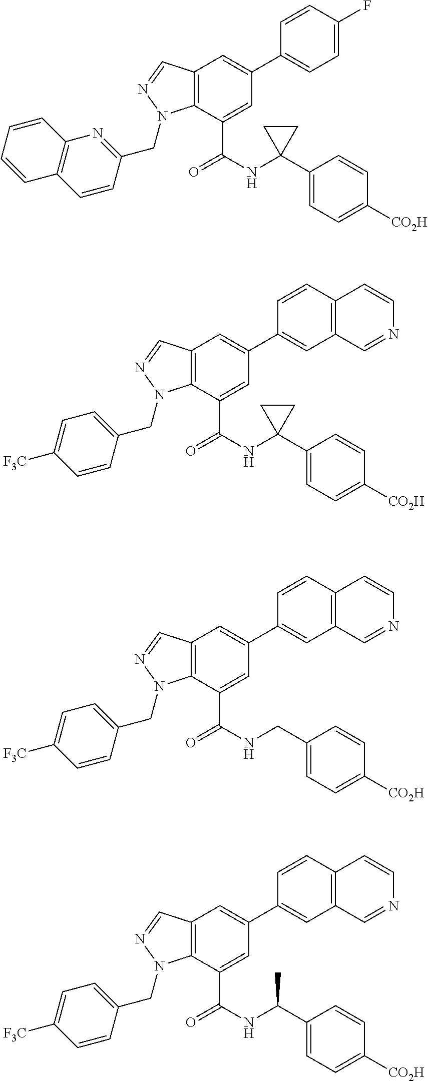 [SMILES: O=C(O)c1ccc(C2(NC(=O)c3cc(-c4ccc(F)cc4)cc4cnn(Cc5ccc6ccccc6n5)c34)CC2)cc1 ; O=C(O)c1ccc(C2(NC(=O)c3cc(-c4ccc5ccncc5c4)cc4cnn(Cc5ccc(C(F)(F)F)cc5)c34)CC2)cc1 ; O=C(O)c1ccc(CNC(=O)c2cc(-c3ccc4ccncc4c3)cc3cnn(Cc4ccc(C(F)(F)F)cc4)c23)cc1 ; C[C@H](NC(=O)c1cc(-c2ccc3ccncc3c2)cc2cnn(Cc3ccc(C(F)(F)F)cc3)c12)c1ccc(C(=O)O)cc1]

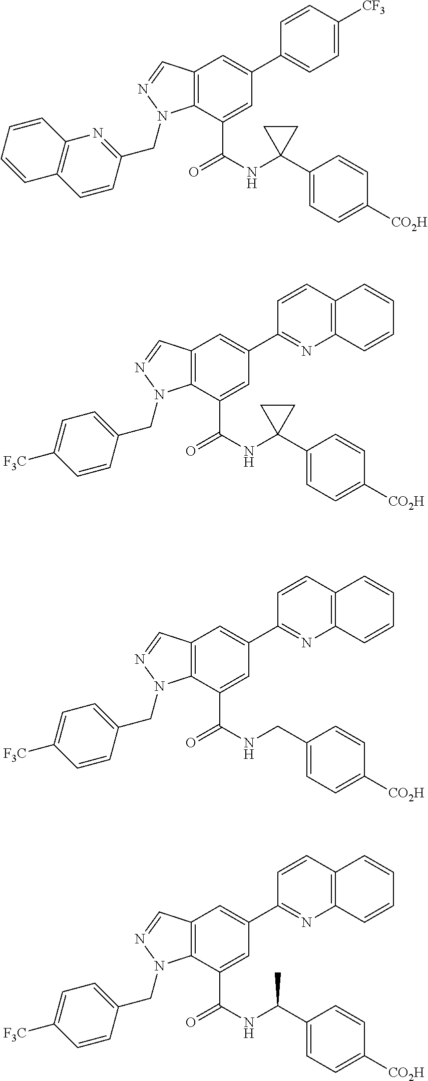 [SMILES: O=C(O)c1ccc(C2(NC(=O)c3cc(-c4ccc(C(F)(F)F)cc4)cc4cnn(Cc5ccc6ccccc6n5)c34)CC2)cc1 ; O=C(O)c1ccc(C2(NC(=O)c3cc(-c4ccc5ccccc5n4)cc4cnn(Cc5ccc(C(F)(F)F)cc5)c34)CC2)cc1 ; O=C(O)c1ccc(CNC(=O)c2cc(-c3ccc4ccccc4n3)cc3cnn(Cc4ccc(C(F)(F)F)cc4)c23)cc1 ; C[C@H](NC(=O)c1cc(-c2ccc3ccccc3n2)cc2cnn(Cc3ccc(C(F)(F)F)cc3)c12)c1ccc(C(=O)O)cc1]

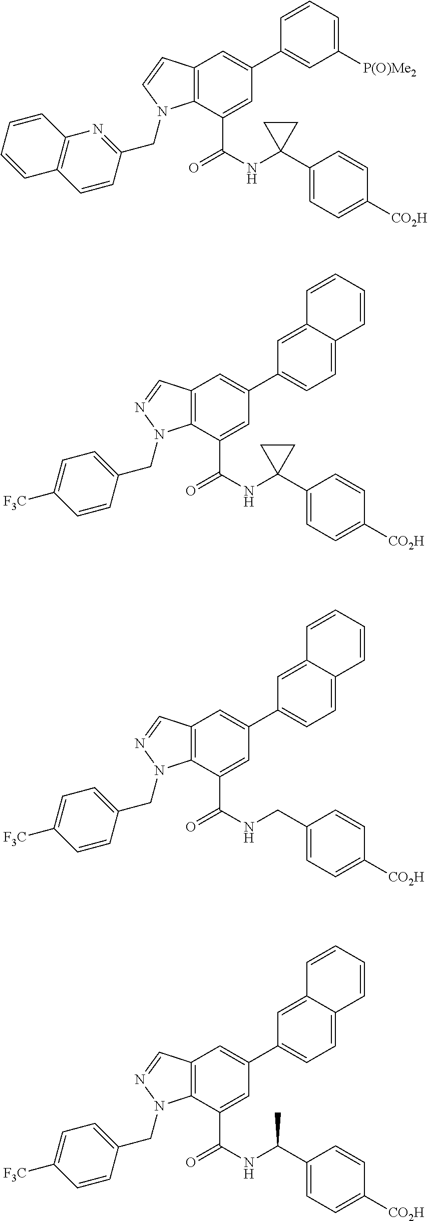

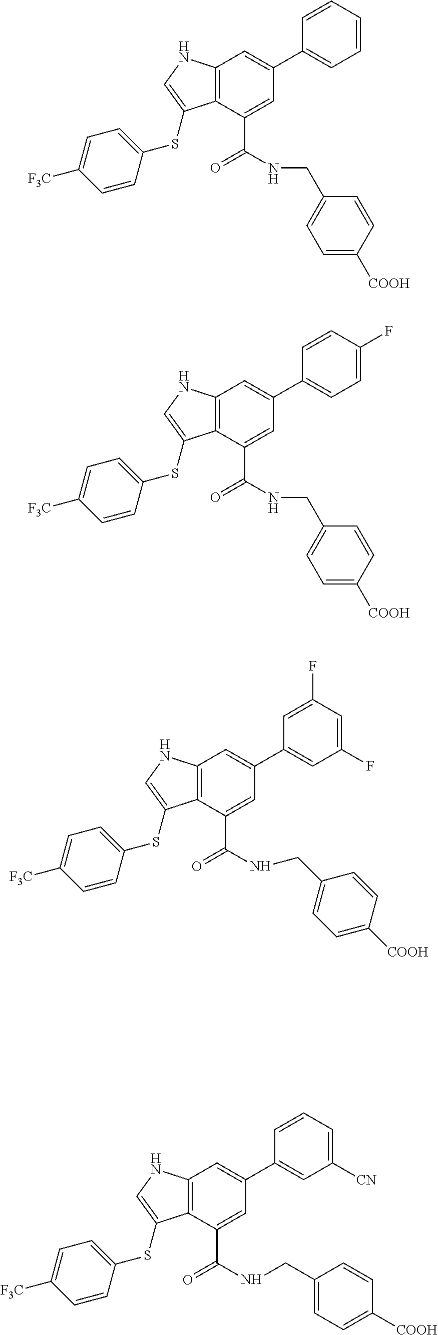 [SMILES: O=C(O)c1ccc(CNC(=O)c2cc(-c3ccccc3)cc3[nH]cc(Sc4ccc(C(F)(F)F)cc4)c23)cc1 ; O=C(O)c1ccc(CNC(=O)c2cc(-c3ccc(F)cc3)cc3[nH]cc(Sc4ccc(C(F)(F)F)cc4)c23)cc1 ; O=C(O)c1ccc(CNC(=O)c2cc(-c3cc(F)cc(F)c3)cc3[nH]cc(Sc4ccc(C(F)(F)F)cc4)c23)cc1 ; N#Cc1cccc(-c2cc(C(=O)NCc3ccc(C(=O)O)cc3)c3c(Sc4ccc(C(F)(F)F)cc4)c[nH]c3c2)c1]

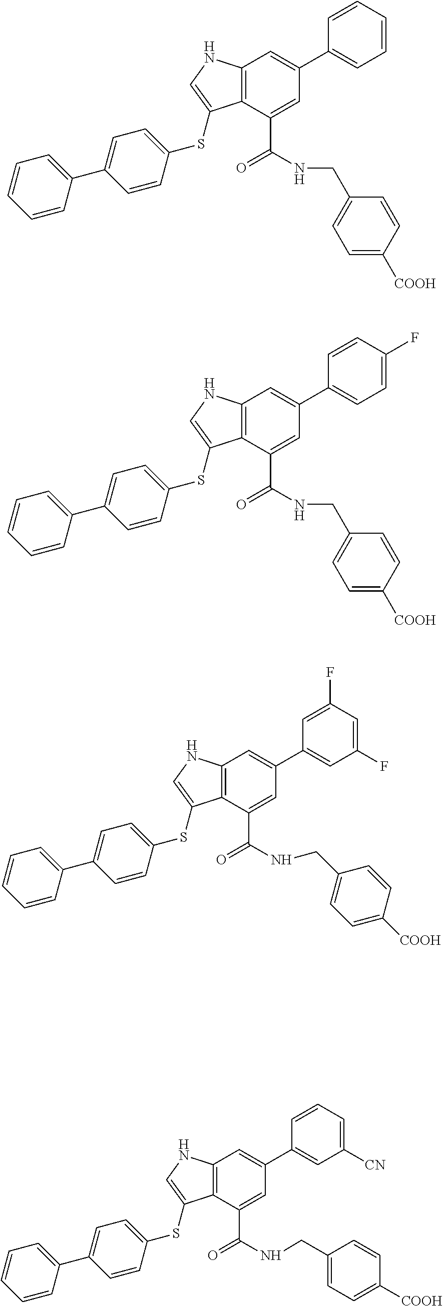 [SMILES: O=C(O)c1ccc(CNC(=O)c2cc(-c3ccccc3)cc3[nH]cc(Sc4ccc(-c5ccccc5)cc4)c23)cc1 ; O=C(O)c1ccc(CNC(=O)c2cc(-c3ccc(F)cc3)cc3[nH]cc(Sc4ccc(-c5ccccc5)cc4)c23)cc1 ; O=C(O)c1ccc(CNC(=O)c2cc(-c3cc(F)cc(F)c3)cc3[nH]cc(Sc4ccc(-c5ccccc5)cc4)c23)cc1 ; N#Cc1cccc(-c2cc(C(=O)NCc3ccc(C(=O)O)cc3)c3c(Sc4ccc(-c5ccccc5)cc4)c[nH]c3c2)c1]

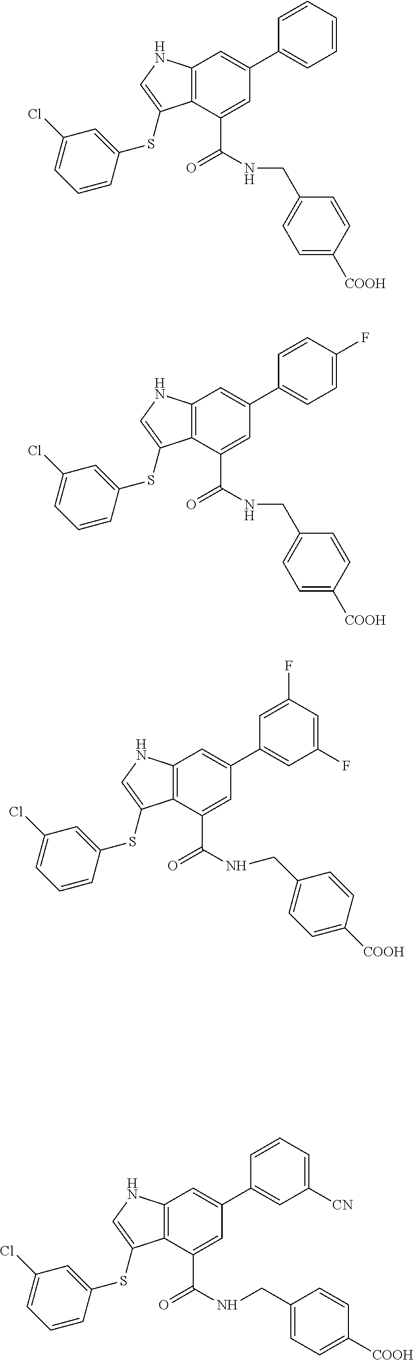 [SMILES: O=C(O)c1ccc(CNC(=O)c2cc(-c3ccccc3)cc3[nH]cc(Sc4cccc(Cl)c4)c23)cc1 ; O=C(O)c1ccc(CNC(=O)c2cc(-c3ccc(F)cc3)cc3[nH]cc(Sc4cccc(Cl)c4)c23)cc1 ; O=C(O)c1ccc(CNC(=O)c2cc(-c3cc(F)cc(F)c3)cc3[nH]cc(Sc4cccc(Cl)c4)c23)cc1 ; N#Cc1cccc(-c2cc(C(=O)NCc3ccc(C(=O)O)cc3)c3c(Sc4cccc(Cl)c4)c[nH]c3c2)c1]

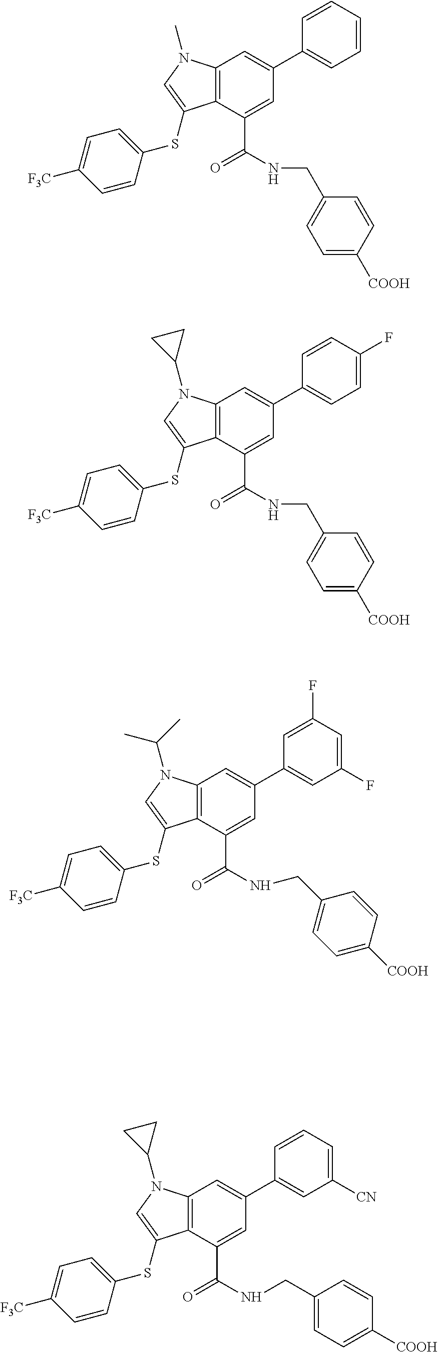 [SMILES: Cn1cc(Sc2ccc(C(F)(F)F)cc2)c2c(C(=O)NCc3ccc(C(=O)O)cc3)cc(-c3ccccc3)cc21 ; O=C(O)c1ccc(CNC(=O)c2cc(-c3ccc(F)cc3)cc3c2c(Sc2ccc(C(F)(F)F)cc2)cn3C2CC2)cc1 ; CC(C)n1cc(Sc2ccc(C(F)(F)F)cc2)c2c(C(=O)NCc3ccc(C(=O)O)cc3)cc(-c3cc(F)cc(F)c3)cc21 ; N#Cc1cccc(-c2cc(C(=O)NCc3ccc(C(=O)O)cc3)c3c(Sc4ccc(C(F)(F)F)cc4)cn(C4CC4)c3c2)c1]

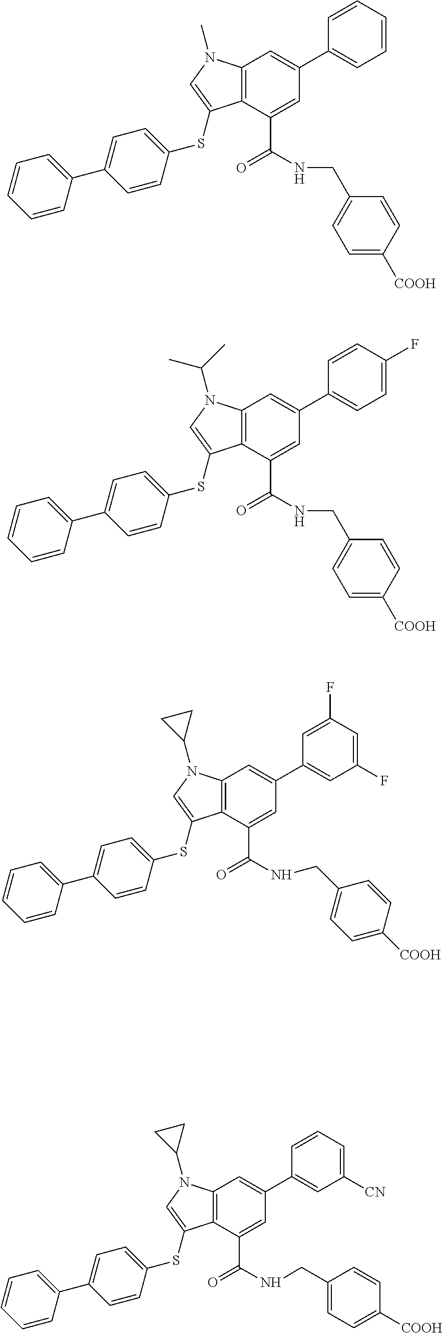 [SMILES: Cn1cc(Sc2ccc(-c3ccccc3)cc2)c2c(C(=O)NCc3ccc(C(=O)O)cc3)cc(-c3ccccc3)cc21 ; CC(C)n1cc(Sc2ccc(-c3ccccc3)cc2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Sc2ccc(-c4ccccc4)cc2)cn3C2CC2)cc1 ; N#Cc1cccc(-c2cc(C(=O)NCc3ccc(C(=O)O)cc3)c3c(Sc4ccc(-c5ccccc5)cc4)cn(C4CC4)c3c2)c1]

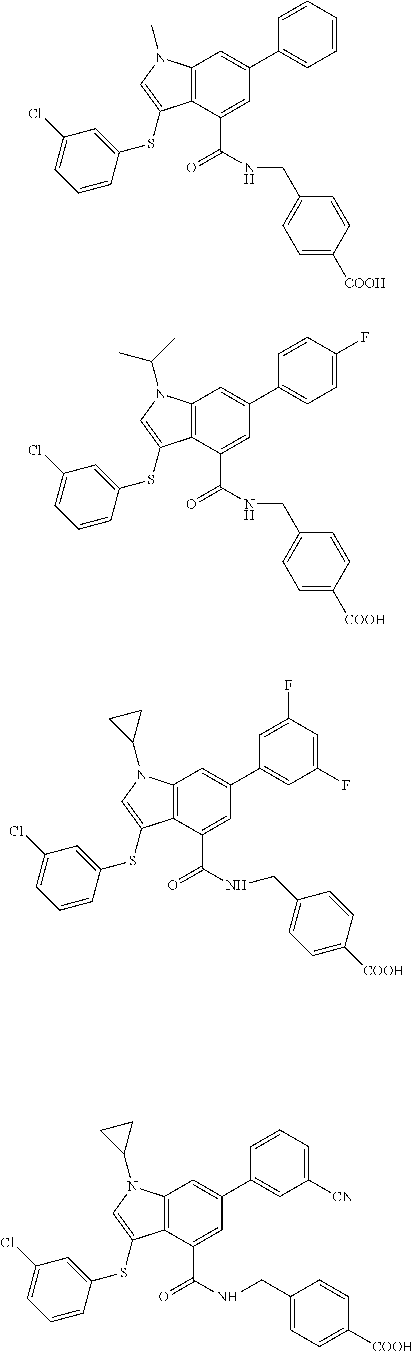 [SMILES: Cn1cc(Sc2cccc(Cl)c2)c2c(C(=O)NCc3ccc(C(=O)O)cc3)cc(-c3ccccc3)cc21 ; CC(C)n1cc(Sc2cccc(Cl)c2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Sc2cccc(Cl)c2)cn3C2CC2)cc1 ; N#Cc1cccc(-c2cc(C(=O)NCc3ccc(C(=O)O)cc3)c3c(Sc4cccc(Cl)c4)cn(C4CC4)c3c2)c1]

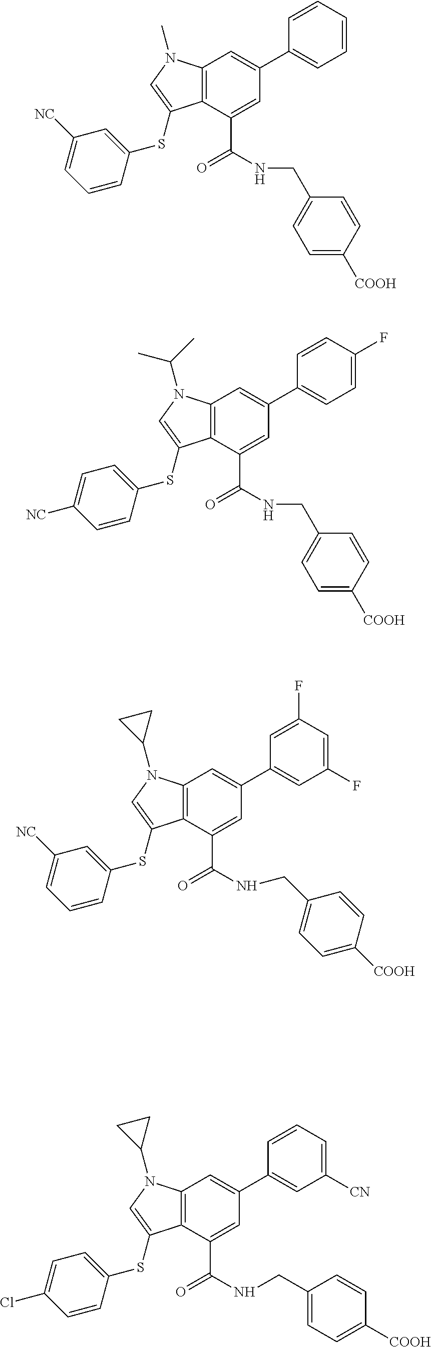 [SMILES: Cn1cc(Sc2cccc(C#N)c2)c2c(C(=O)NCc3ccc(C(=O)O)cc3)cc(-c3ccccc3)cc21 ; CC(C)n1cc(Sc2ccc(C#N)cc2)c2c(C(=O)NCc3ccc(C(=O)O)cc3)cc(-c3ccc(F)cc3)cc21 ; N#Cc1cccc(Sc2cn(C3CC3)c3cc(-c4cc(F)cc(F)c4)cc(C(=O)NCc4ccc(C(=O)O)cc4)c23)c1 ; N#Cc1cccc(-c2cc(C(=O)NCc3ccc(C(=O)O)cc3)c3c(Sc4ccc(Cl)cc4)cn(C4CC4)c3c2)c1]

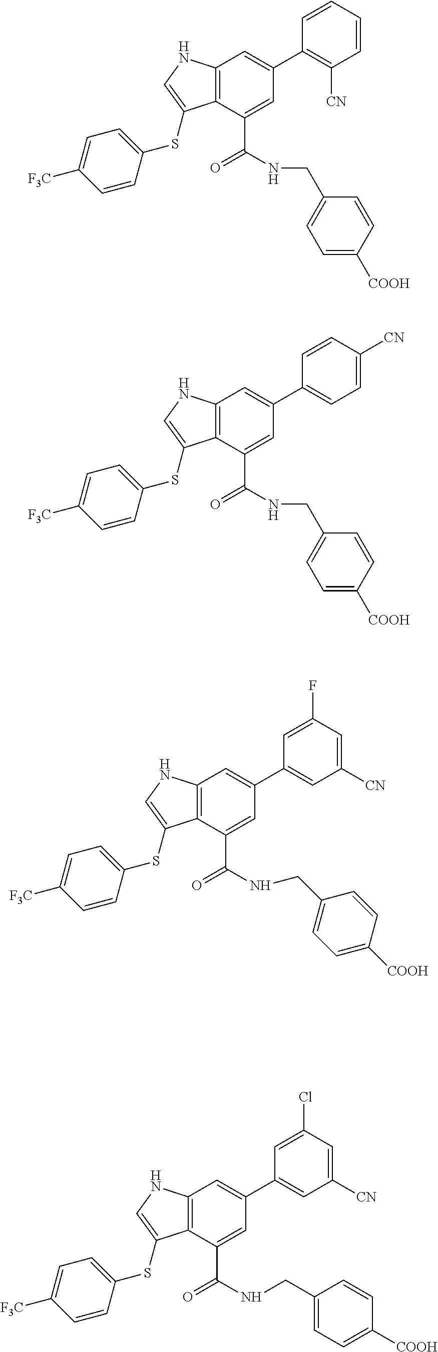 [SMILES: N#Cc1ccccc1-c1cc(C(=O)NCc2ccc(C(=O)O)cc2)c2c(Sc3ccc(C(F)(F)F)cc3)c[nH]c2c1 ; N#Cc1ccc(-c2cc(C(=O)NCc3ccc(C(=O)O)cc3)c3c(Sc4ccc(C(F)(F)F)cc4)c[nH]c3c2)cc1 ; N#Cc1cc(F)cc(-c2cc(C(=O)NCc3ccc(C(=O)O)cc3)c3c(Sc4ccc(C(F)(F)F)cc4)c[nH]c3c2)c1 ; N#Cc1cc(Cl)cc(-c2cc(C(=O)NCc3ccc(C(=O)O)cc3)c3c(Sc4ccc(C(F)(F)F)cc4)c[nH]c3c2)c1]

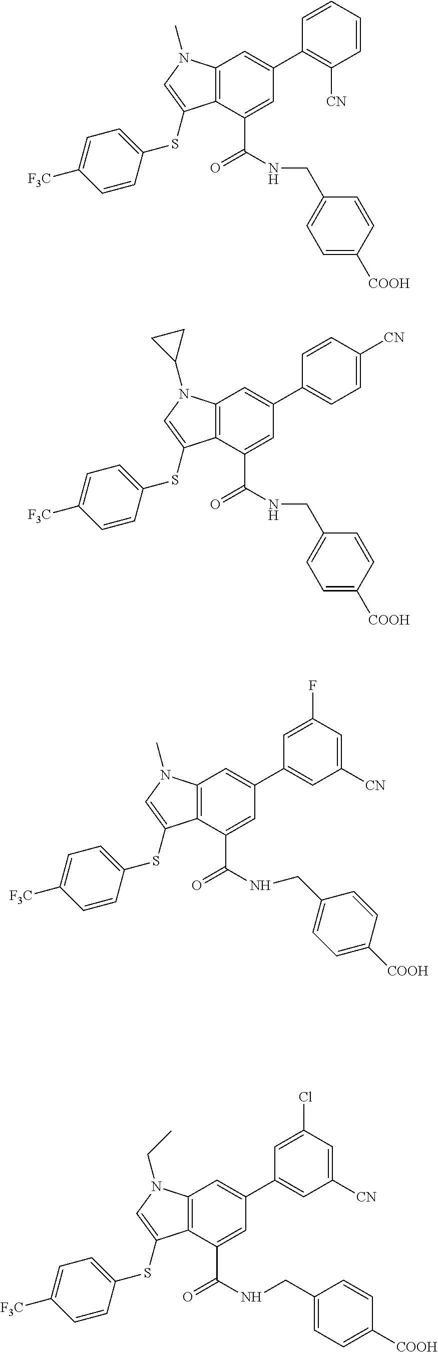 [SMILES: Cn1cc(Sc2ccc(C(F)(F)F)cc2)c2c(C(=O)NCc3ccc(C(=O)O)cc3)cc(-c3ccccc3C#N)cc21 ; N#Cc1ccc(-c2cc(C(=O)NCc3ccc(C(=O)O)cc3)c3c(Sc4ccc(C(F)(F)F)cc4)cn(C4CC4)c3c2)cc1 ; Cn1cc(Sc2ccc(C(F)(F)F)cc2)c2c(C(=O)NCc3ccc(C(=O)O)cc3)cc(-c3cc(F)cc(C#N)c3)cc21 ; CCn1cc(Sc2ccc(C(F)(F)F)cc2)c2c(C(=O)NCc3ccc(C(=O)O)cc3)cc(-c3cc(Cl)cc(C#N)c3)cc21]

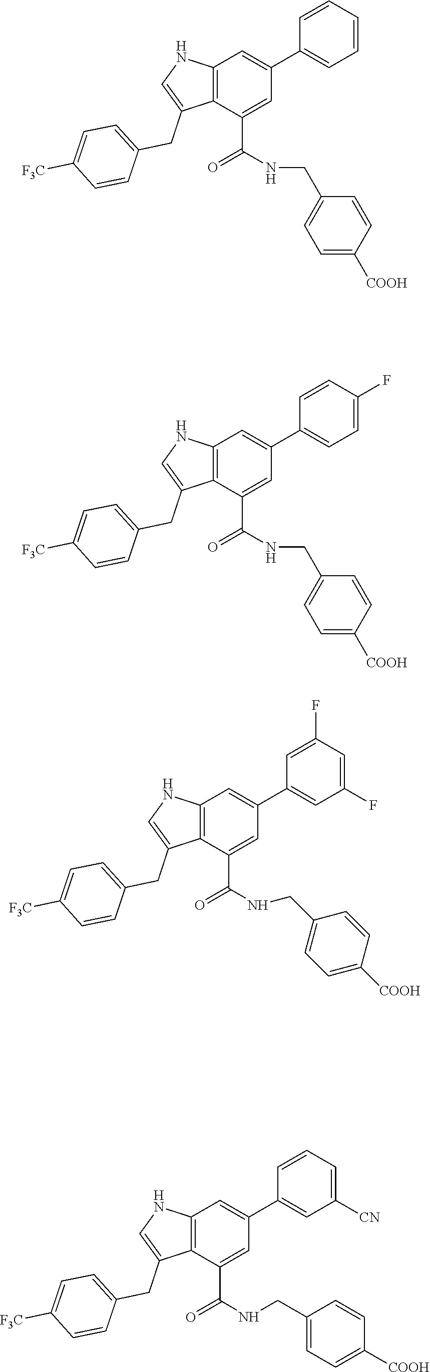 [SMILES: O=C(O)c1ccc(CNC(=O)c2cc(-c3ccccc3)cc3[nH]cc(Cc4ccc(C(F)(F)F)cc4)c23)cc1 ; O=C(O)c1ccc(CNC(=O)c2cc(-c3ccc(F)cc3)cc3[nH]cc(Cc4ccc(C(F)(F)F)cc4)c23)cc1 ; O=C(O)c1ccc(CNC(=O)c2cc(-c3cc(F)cc(F)c3)cc3[nH]cc(Cc4ccc(C(F)(F)F)cc4)c23)cc1 ; N#Cc1cccc(-c2cc(C(=O)NCc3ccc(C(=O)O)cc3)c3c(Cc4ccc(C(F)(F)F)cc4)c[nH]c3c2)c1]

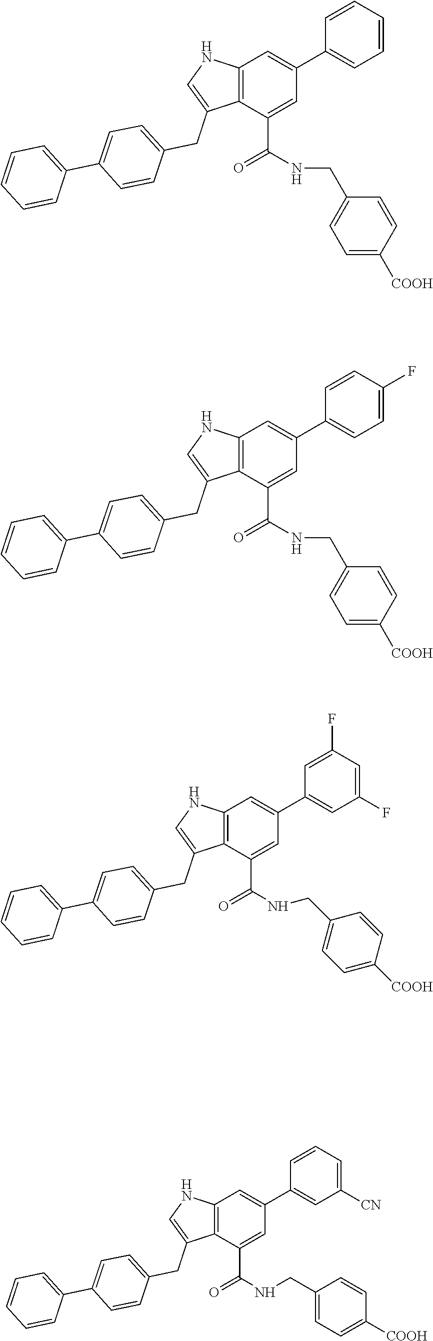 [SMILES: O=C(O)c1ccc(CNC(=O)c2cc(-c3ccccc3)cc3[nH]cc(Cc4ccc(-c5ccccc5)cc4)c23)cc1 ; O=C(O)c1ccc(CNC(=O)c2cc(-c3ccc(F)cc3)cc3[nH]cc(Cc4ccc(-c5ccccc5)cc4)c23)cc1 ; O=C(O)c1ccc(CNC(=O)c2cc(-c3cc(F)cc(F)c3)cc3[nH]cc(Cc4ccc(-c5ccccc5)cc4)c23)cc1 ; N#Cc1cccc(-c2cc(C(=O)NCc3ccc(C(=O)O)cc3)c3c(Cc4ccc(-c5ccccc5)cc4)c[nH]c3c2)c1]

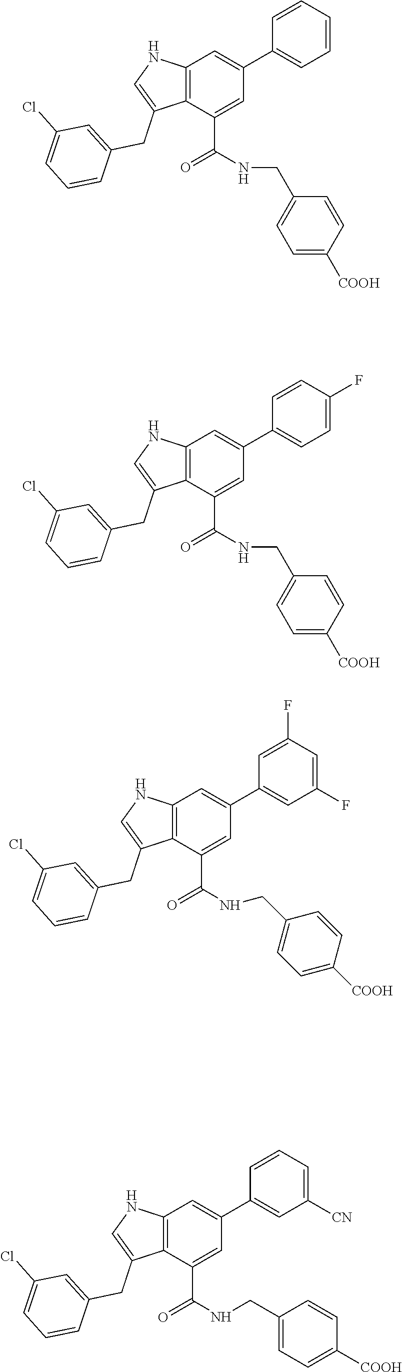 [SMILES: O=C(O)c1ccc(CNC(=O)c2cc(-c3ccccc3)cc3[nH]cc(Cc4cccc(Cl)c4)c23)cc1 ; O=C(O)c1ccc(CNC(=O)c2cc(-c3ccc(F)cc3)cc3[nH]cc(Cc4cccc(Cl)c4)c23)cc1 ; O=C(O)c1ccc(CNC(=O)c2cc(-c3cc(F)cc(F)c3)cc3[nH]cc(Cc4cccc(Cl)c4)c23)cc1 ; N#Cc1cccc(-c2cc(C(=O)NCc3ccc(C(=O)O)cc3)c3c(Cc4cccc(Cl)c4)c[nH]c3c2)c1]

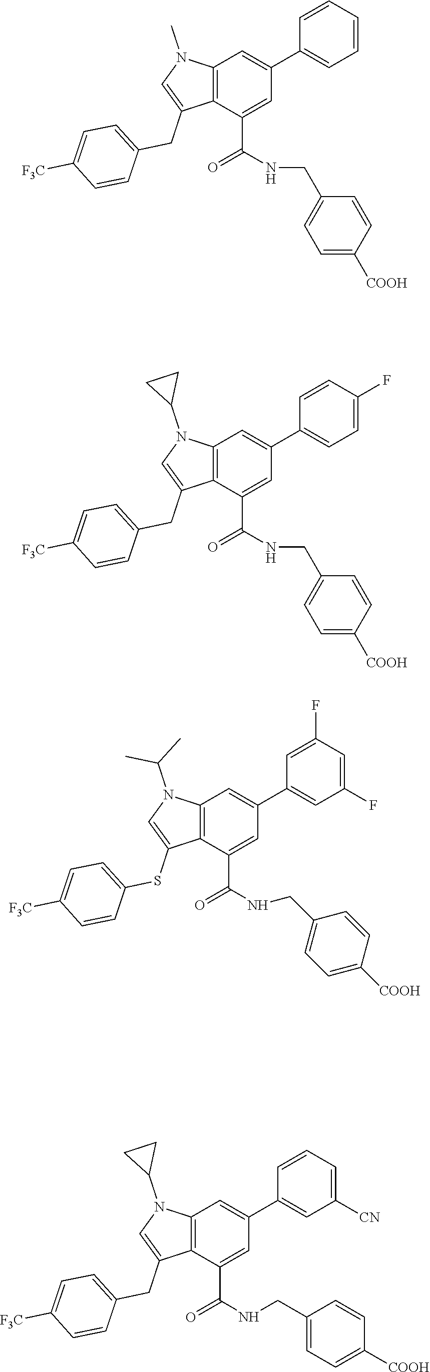 [SMILES: Cn1cc(Cc2ccc(C(F)(F)F)cc2)c2c(C(=O)NCc3ccc(C(=O)O)cc3)cc(-c3ccccc3)cc21 ; O=C(O)c1ccc(CNC(=O)c2cc(-c3ccc(F)cc3)cc3c2c(Cc2ccc(C(F)(F)F)cc2)cn3C2CC2)cc1 ; CC(C)n1cc(Sc2ccc(C(F)(F)F)cc2)c2c(C(=O)NCc3ccc(C(=O)O)cc3)cc(-c3cc(F)cc(F)c3)cc21 ; N#Cc1cccc(-c2cc(C(=O)NCc3ccc(C(=O)O)cc3)c3c(Cc4ccc(C(F)(F)F)cc4)cn(C4CC4)c3c2)c1]

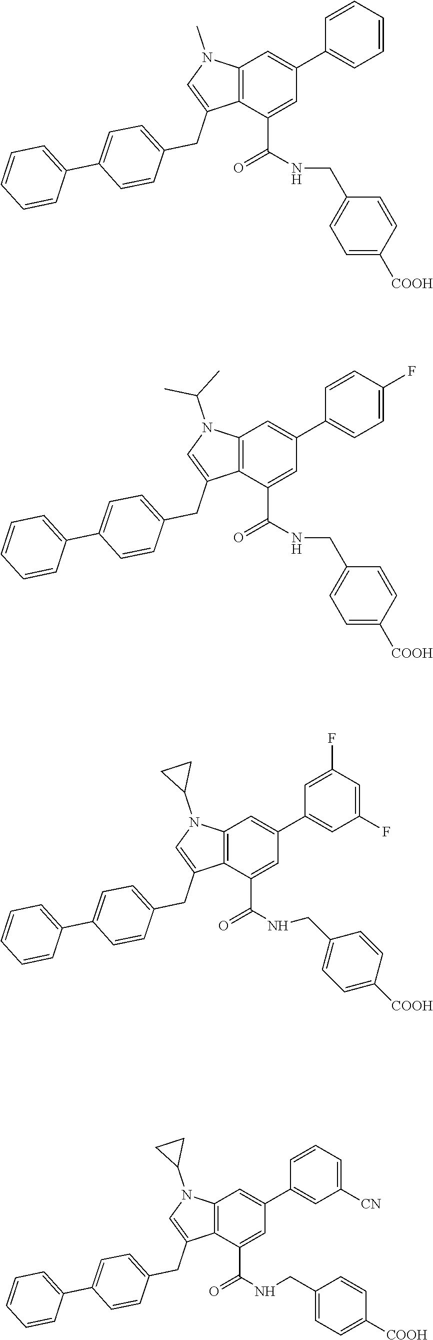 [SMILES: Cn1cc(Cc2ccc(-c3ccccc3)cc2)c2c(C(=O)NCc3ccc(C(=O)O)cc3)cc(-c3ccccc3)cc21 ; CC(C)n1cc(Cc2ccc(-c3ccccc3)cc2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Cc2ccc(-c4ccccc4)cc2)cn3C2CC2)cc1 ; N#Cc1cccc(-c2cc(C(=O)NCc3ccc(C(=O)O)cc3)c3c(Cc4ccc(-c5ccccc5)cc4)cn(C4CC4)c3c2)c1]

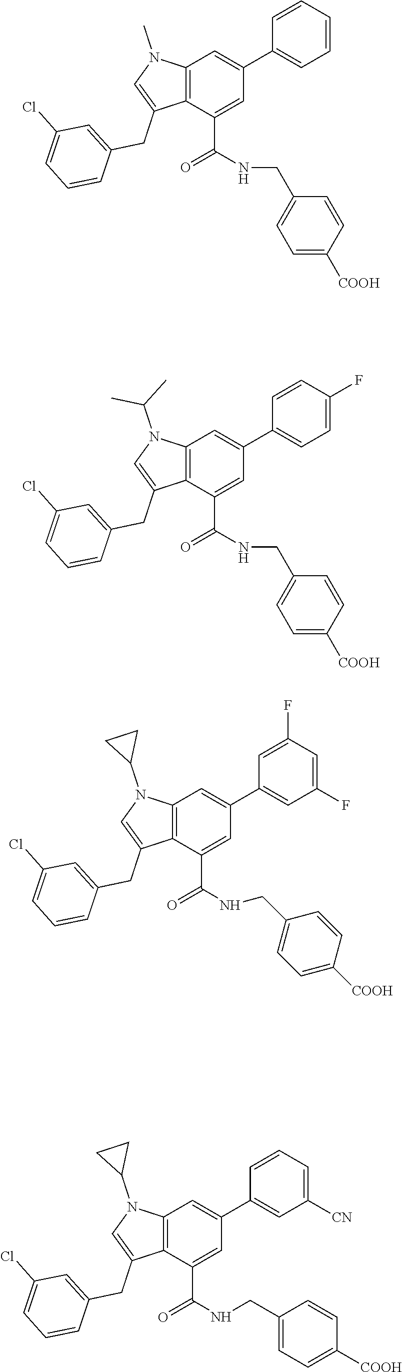 [SMILES: Cn1cc(Cc2cccc(Cl)c2)c2c(C(=O)NCc3ccc(C(=O)O)cc3)cc(-c3ccccc3)cc21 ; CC(C)n1cc(Cc2cccc(Cl)c2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Cc2cccc(Cl)c2)cn3C2CC2)cc1 ; N#Cc1cccc(-c2cc(C(=O)NCc3ccc(C(=O)O)cc3)c3c(Cc4cccc(Cl)c4)cn(C4CC4)c3c2)c1]

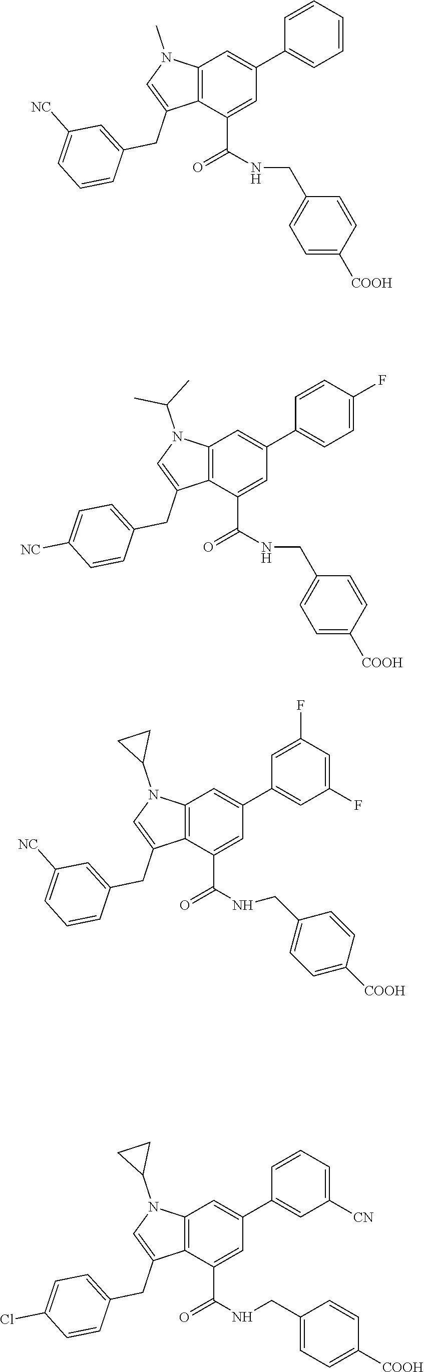 [SMILES: Cn1cc(Cc2cccc(C#N)c2)c2c(C(=O)NCc3ccc(C(=O)O)cc3)cc(-c3ccccc3)cc21 ; CC(C)n1cc(Cc2ccc(C#N)cc2)c2c(C(=O)NCc3ccc(C(=O)O)cc3)cc(-c3ccc(F)cc3)cc21 ; N#Cc1cccc(Cc2cn(C3CC3)c3cc(-c4cc(F)cc(F)c4)cc(C(=O)NCc4ccc(C(=O)O)cc4)c23)c1 ; N#Cc1cccc(-c2cc(C(=O)NCc3ccc(C(=O)O)cc3)c3c(Cc4ccc(Cl)cc4)cn(C4CC4)c3c2)c1]

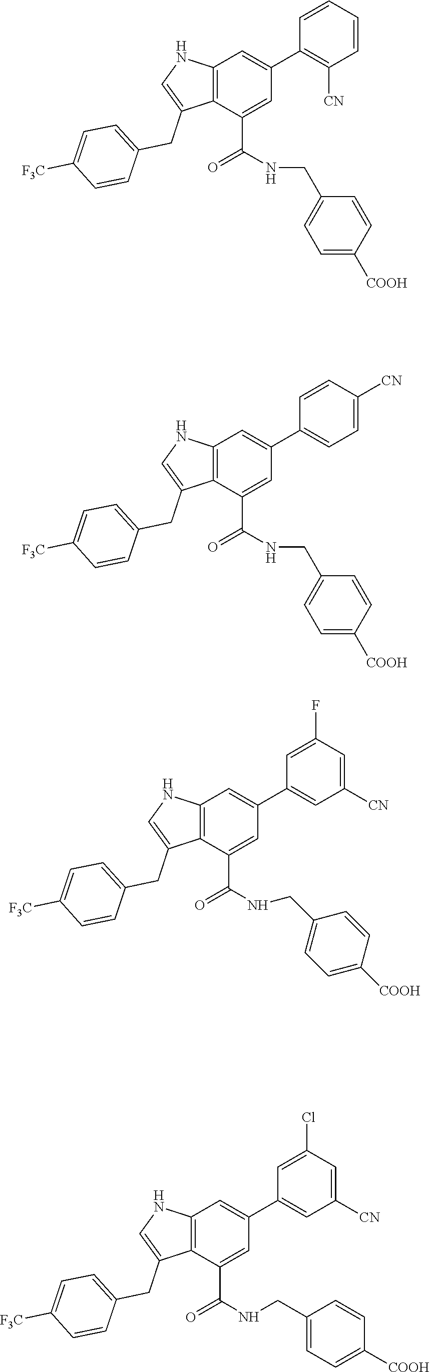 [SMILES: N#Cc1ccccc1-c1cc(C(=O)NCc2ccc(C(=O)O)cc2)c2c(Cc3ccc(C(F)(F)F)cc3)c[nH]c2c1 ; N#Cc1ccc(-c2cc(C(=O)NCc3ccc(C(=O)O)cc3)c3c(Cc4ccc(C(F)(F)F)cc4)c[nH]c3c2)cc1 ; N#Cc1cc(F)cc(-c2cc(C(=O)NCc3ccc(C(=O)O)cc3)c3c(Cc4ccc(C(F)(F)F)cc4)c[nH]c3c2)c1 ; N#Cc1cc(Cl)cc(-c2cc(C(=O)NCc3ccc(C(=O)O)cc3)c3c(Cc4ccc(C(F)(F)F)cc4)c[nH]c3c2)c1]

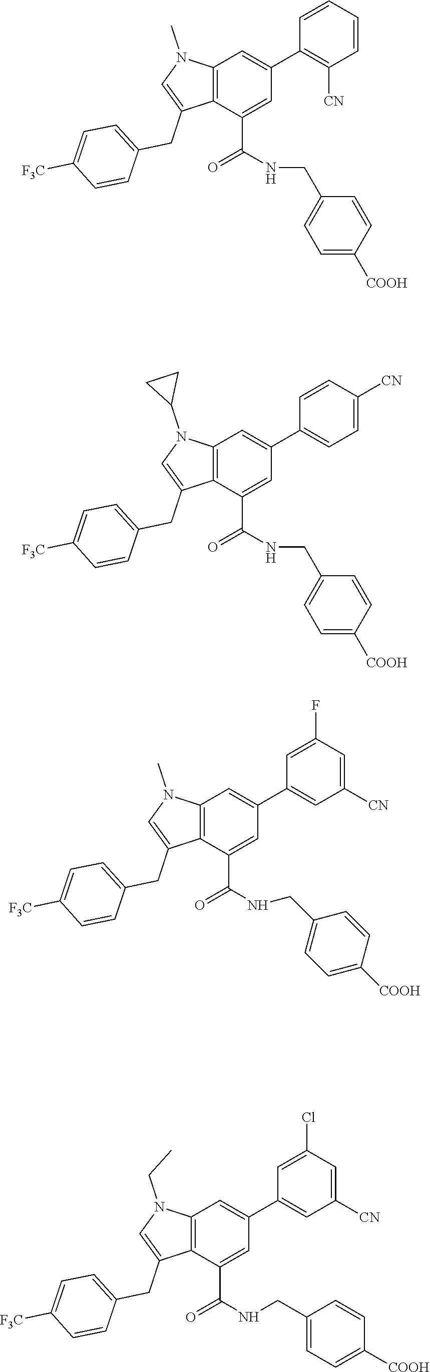 [SMILES: Cn1cc(Cc2ccc(C(F)(F)F)cc2)c2c(C(=O)NCc3ccc(C(=O)O)cc3)cc(-c3ccccc3C#N)cc21 ; N#Cc1ccc(-c2cc(C(=O)NCc3ccc(C(=O)O)cc3)c3c(Cc4ccc(C(F)(F)F)cc4)cn(C4CC4)c3c2)cc1 ; Cn1cc(Cc2ccc(C(F)(F)F)cc2)c2c(C(=O)NCc3ccc(C(=O)O)cc3)cc(-c3cc(F)cc(C#N)c3)cc21 ; CCn1cc(Cc2ccc(C(F)(F)F)cc2)c2c(C(=O)NCc3ccc(C(=O)O)cc3)cc(-c3cc(Cl)cc(C#N)c3)cc21]

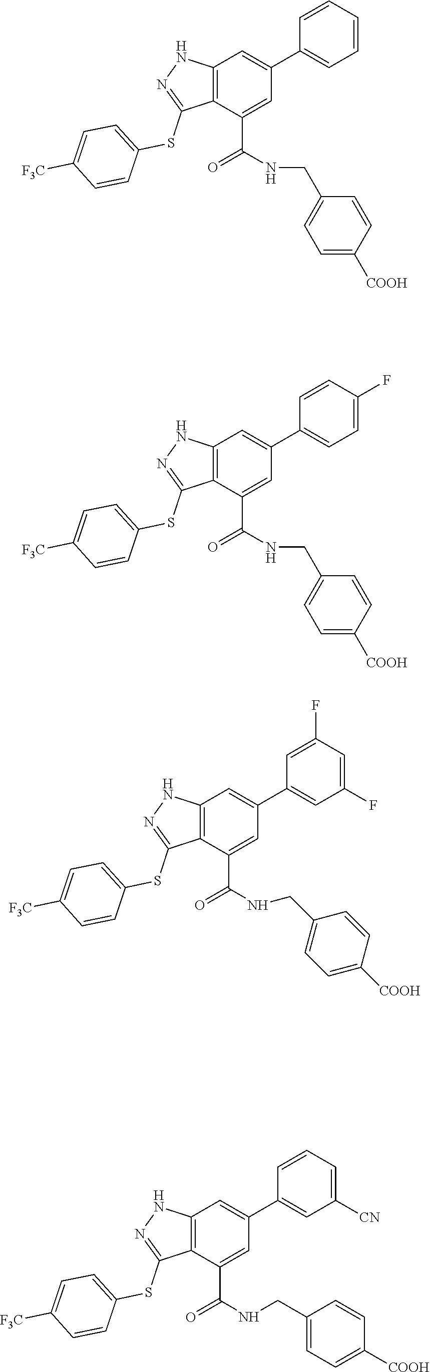 [SMILES: O=C(O)c1ccc(CNC(=O)c2cc(-c3ccccc3)cc3[nH]nc(Sc4ccc(C(F)(F)F)cc4)c23)cc1 ; O=C(O)c1ccc(CNC(=O)c2cc(-c3ccc(F)cc3)cc3[nH]nc(Sc4ccc(C(F)(F)F)cc4)c23)cc1 ; O=C(O)c1ccc(CNC(=O)c2cc(-c3cc(F)cc(F)c3)cc3[nH]nc(Sc4ccc(C(F)(F)F)cc4)c23)cc1 ; N#Cc1cccc(-c2cc(C(=O)NCc3ccc(C(=O)O)cc3)c3c(Sc4ccc(C(F)(F)F)cc4)n[nH]c3c2)c1]

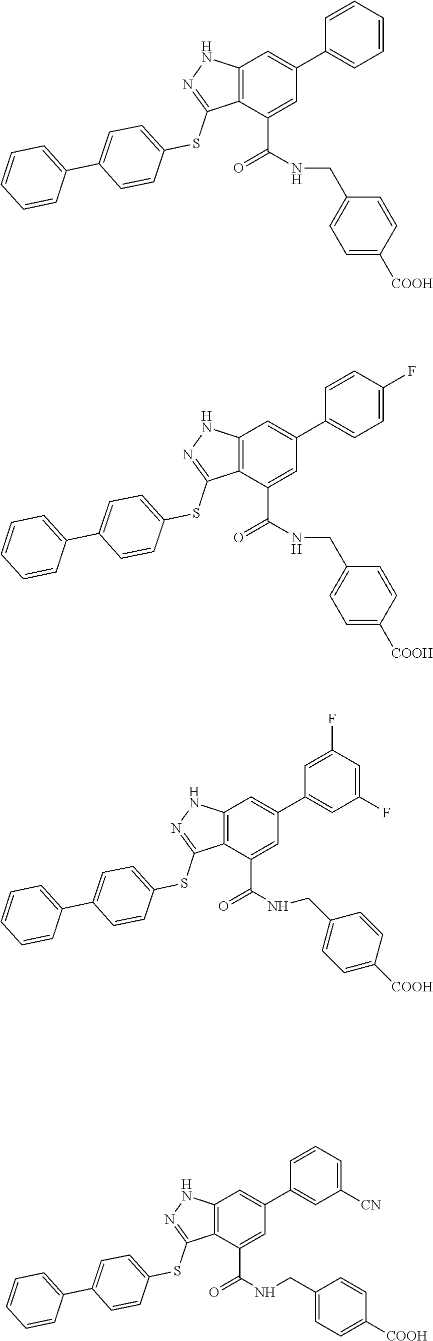 [SMILES: O=C(O)c1ccc(CNC(=O)c2cc(-c3ccccc3)cc3[nH]nc(Sc4ccc(-c5ccccc5)cc4)c23)cc1 ; O=C(O)c1ccc(CNC(=O)c2cc(-c3ccc(F)cc3)cc3[nH]nc(Sc4ccc(-c5ccccc5)cc4)c23)cc1 ; O=C(O)c1ccc(CNC(=O)c2cc(-c3cc(F)cc(F)c3)cc3[nH]nc(Sc4ccc(-c5ccccc5)cc4)c23)cc1 ; N#Cc1cccc(-c2cc(C(=O)NCc3ccc(C(=O)O)cc3)c3c(Sc4ccc(-c5ccccc5)cc4)n[nH]c3c2)c1]

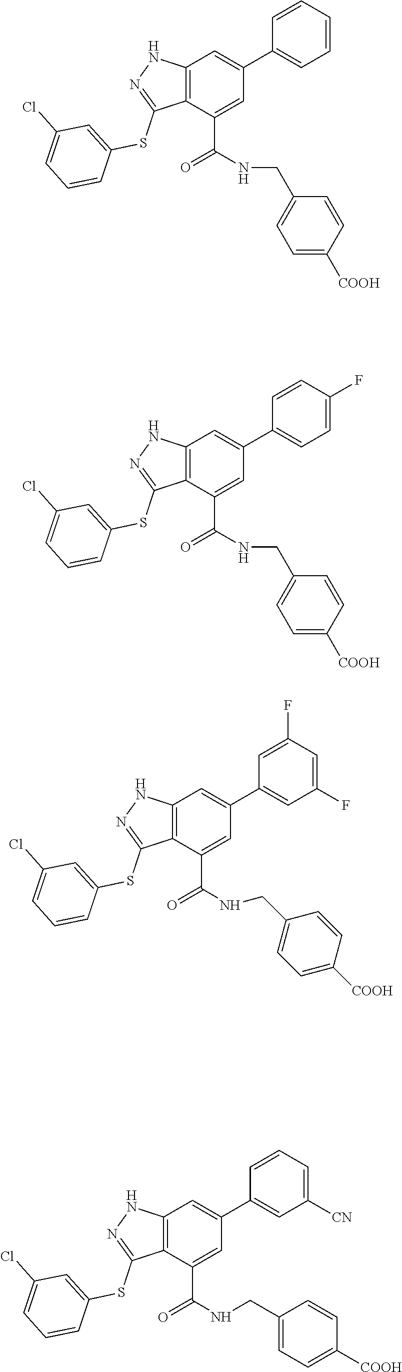 [SMILES: O=C(O)c1ccc(CNC(=O)c2cc(-c3ccccc3)cc3[nH]nc(Sc4cccc(Cl)c4)c23)cc1 ; O=C(O)c1ccc(CNC(=O)c2cc(-c3ccc(F)cc3)cc3[nH]nc(Sc4cccc(Cl)c4)c23)cc1 ; O=C(O)c1ccc(CNC(=O)c2cc(-c3cc(F)cc(F)c3)cc3[nH]nc(Sc4cccc(Cl)c4)c23)cc1 ; N#Cc1cccc(-c2cc(C(=O)NCc3ccc(C(=O)O)cc3)c3c(Sc4cccc(Cl)c4)n[nH]c3c2)c1]

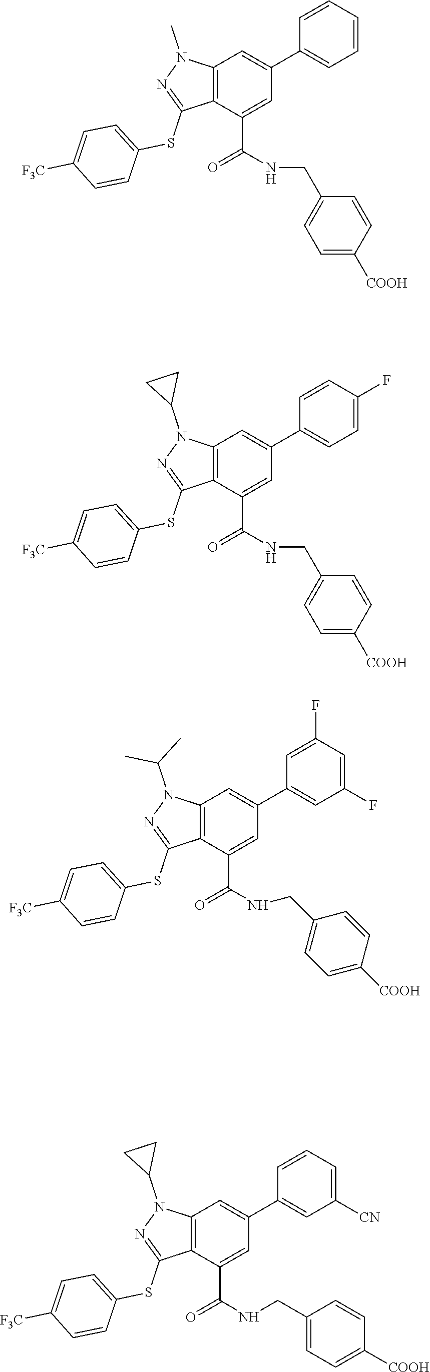 [SMILES: Cn1nc(Sc2ccc(C(F)(F)F)cc2)c2c(C(=O)NCc3ccc(C(=O)O)cc3)cc(-c3ccccc3)cc21 ; O=C(O)c1ccc(CNC(=O)c2cc(-c3ccc(F)cc3)cc3c2c(Sc2ccc(C(F)(F)F)cc2)nn3C2CC2)cc1 ; CC(C)n1nc(Sc2ccc(C(F)(F)F)cc2)c2c(C(=O)NCc3ccc(C(=O)O)cc3)cc(-c3cc(F)cc(F)c3)cc21 ; N#Cc1cccc(-c2cc(C(=O)NCc3ccc(C(=O)O)cc3)c3c(Sc4ccc(C(F)(F)F)cc4)nn(C4CC4)c3c2)c1]

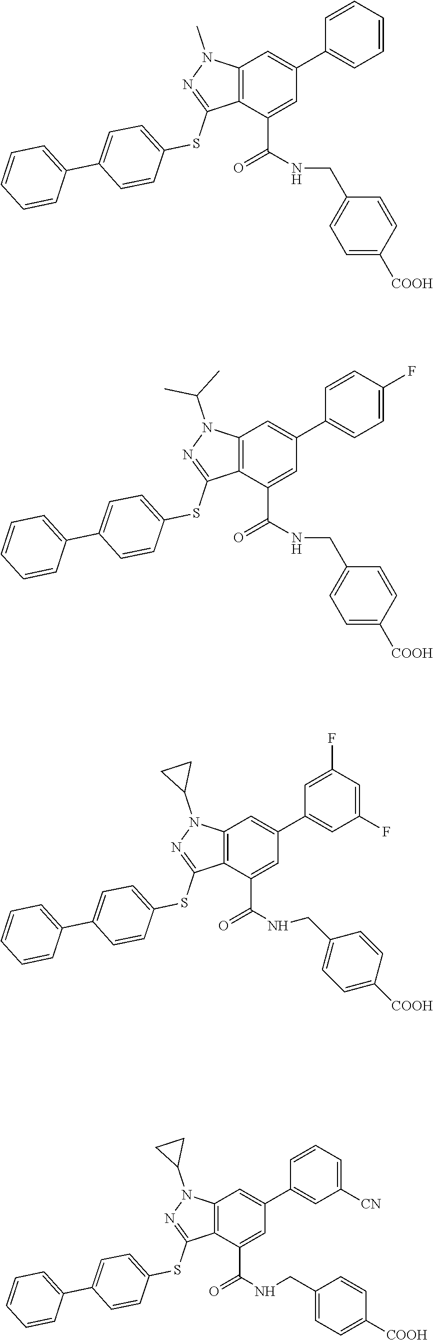 [SMILES: Cn1nc(Sc2ccc(-c3ccccc3)cc2)c2c(C(=O)NCc3ccc(C(=O)O)cc3)cc(-c3ccccc3)cc21 ; CC(C)n1nc(Sc2ccc(-c3ccccc3)cc2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Sc2ccc(-c4ccccc4)cc2)nn3C2CC2)cc1 ; N#Cc1cccc(-c2cc(C(=O)NCc3ccc(C(=O)O)cc3)c3c(Sc4ccc(-c5ccccc5)cc4)nn(C4CC4)c3c2)c1]

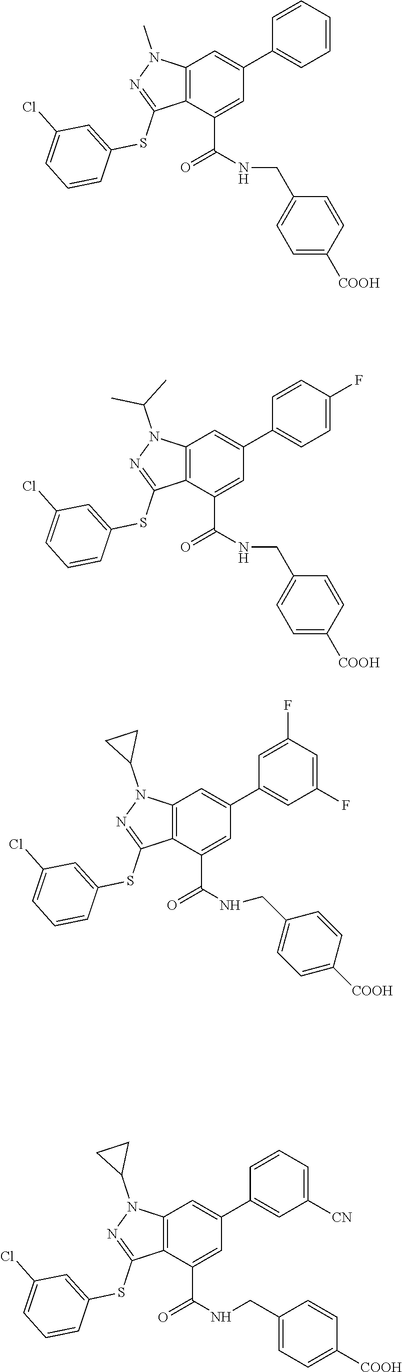 [SMILES: Cn1nc(Sc2cccc(Cl)c2)c2c(C(=O)NCc3ccc(C(=O)O)cc3)cc(-c3ccccc3)cc21 ; CC(C)n1nc(Sc2cccc(Cl)c2)c2c(C(=O)NCc3ccc(C(=O)O)cc3)cc(-c3ccc(F)cc3)cc21 ; O=C(O)c1ccc(CNC(=O)c2cc(-c3cc(F)cc(F)c3)cc3c2c(Sc2cccc(Cl)c2)nn3C2CC2)cc1 ; N#Cc1cccc(-c2cc(C(=O)NCc3ccc(C(=O)O)cc3)c3c(Sc4cccc(Cl)c4)nn(C4CC4)c3c2)c1]

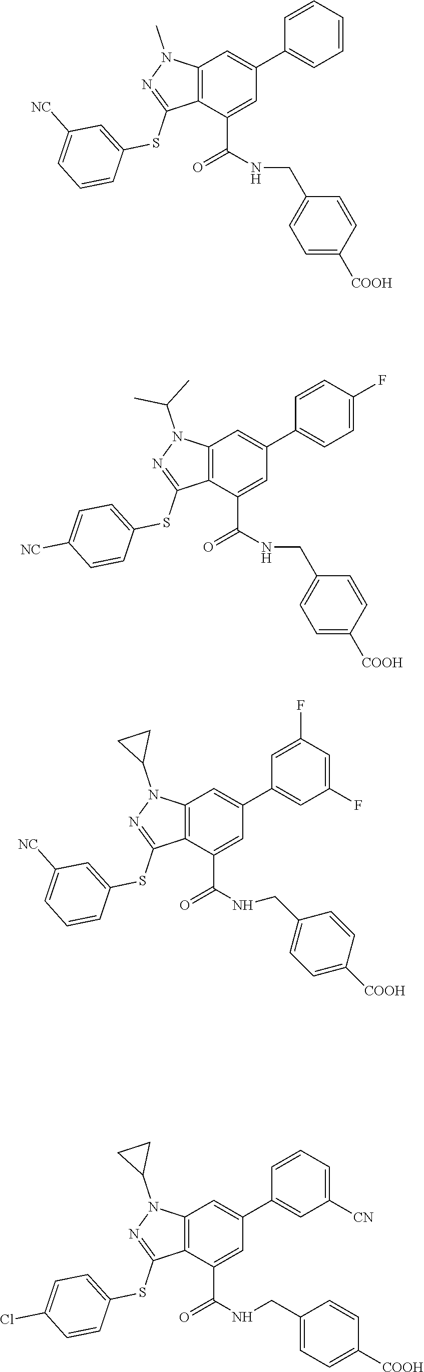 [SMILES: Cn1nc(Sc2cccc(C#N)c2)c2c(C(=O)NCc3ccc(C(=O)O)cc3)cc(-c3ccccc3)cc21 ; CC(C)n1nc(Sc2ccc(C#N)cc2)c2c(C(=O)NCc3ccc(C(=O)O)cc3)cc(-c3ccc(F)cc3)cc21 ; N#Cc1cccc(Sc2nn(C3CC3)c3cc(-c4cc(F)cc(F)c4)cc(C(=O)NCc4ccc(C(=O)O)cc4)c23)c1 ; N#Cc1cccc(-c2cc(C(=O)NCc3ccc(C(=O)O)cc3)c3c(Sc4ccc(Cl)cc4)nn(C4CC4)c3c2)c1]

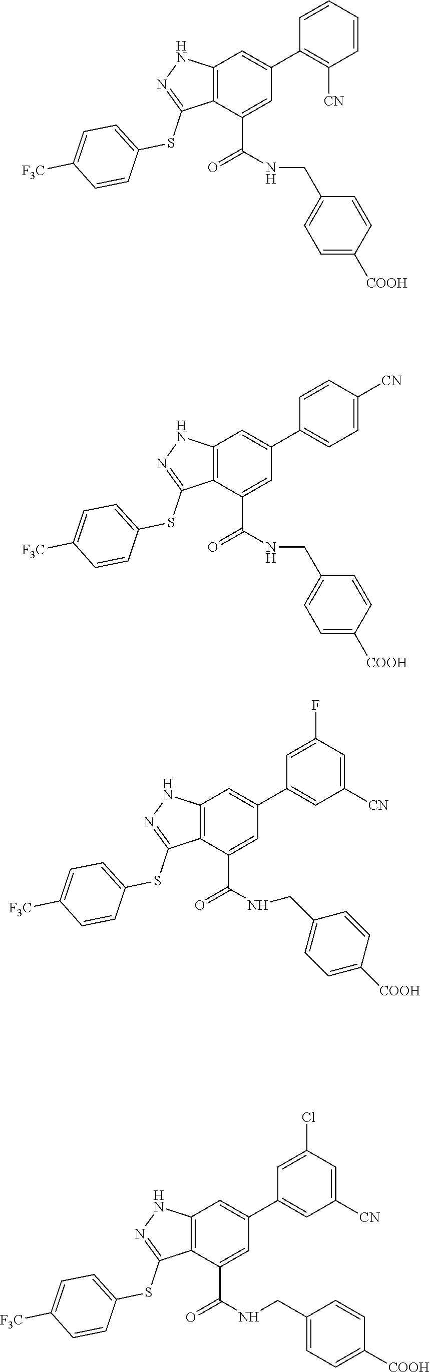 [SMILES: N#Cc1ccccc1-c1cc(C(=O)NCc2ccc(C(=O)O)cc2)c2c(Sc3ccc(C(F)(F)F)cc3)n[nH]c2c1 ; N#Cc1ccc(-c2cc(C(=O)NCc3ccc(C(=O)O)cc3)c3c(Sc4ccc(C(F)(F)F)cc4)n[nH]c3c2)cc1 ; N#Cc1cc(F)cc(-c2cc(C(=O)NCc3ccc(C(=O)O)cc3)c3c(Sc4ccc(C(F)(F)F)cc4)n[nH]c3c2)c1 ; N#Cc1cc(Cl)cc(-c2cc(C(=O)NCc3ccc(C(=O)O)cc3)c3c(Sc4ccc(C(F)(F)F)cc4)n[nH]c3c2)c1]

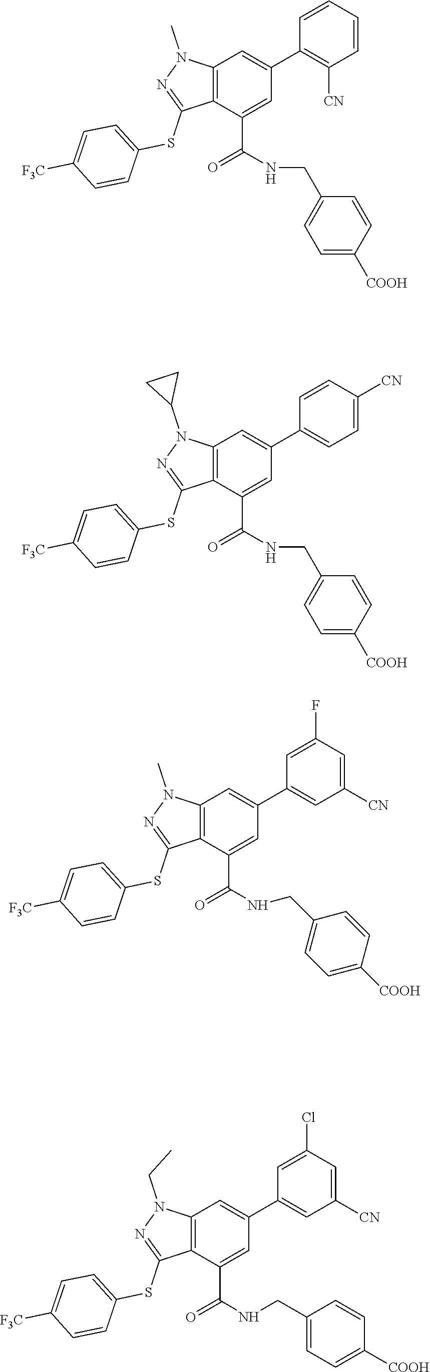 [SMILES: Cn1nc(Sc2ccc(C(F)(F)F)cc2)c2c(C(=O)NCc3ccc(C(=O)O)cc3)cc(-c3ccccc3C#N)cc21 ; N#Cc1ccc(-c2cc(C(=O)NCc3ccc(C(=O)O)cc3)c3c(Sc4ccc(C(F)(F)F)cc4)nn(C4CC4)c3c2)cc1 ; Cn1nc(Sc2ccc(C(F)(F)F)cc2)c2c(C(=O)NCc3ccc(C(=O)O)cc3)cc(-c3cc(F)cc(C#N)c3)cc21 ; CCn1nc(Sc2ccc(C(F)(F)F)cc2)c2c(C(=O)NCc3ccc(C(=O)O)cc3)cc(-c3cc(Cl)cc(C#N)c3)cc21]

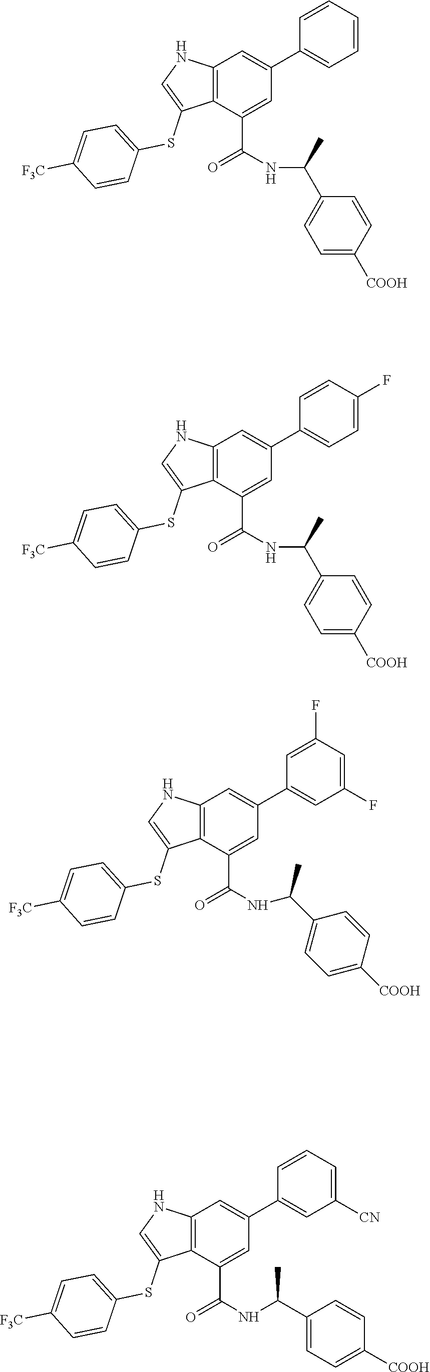 [SMILES: C[C@H](NC(=O)c1cc(-c2ccccc2)cc2[nH]cc(Sc3ccc(C(F)(F)F)cc3)c12)c1ccc(C(=O)O)cc1 ; C[C@H](NC(=O)c1cc(-c2ccc(F)cc2)cc2[nH]cc(Sc3ccc(C(F)(F)F)cc3)c12)c1ccc(C(=O)O)cc1 ; C[C@H](NC(=O)c1cc(-c2cc(F)cc(F)c2)cc2[nH]cc(Sc3ccc(C(F)(F)F)cc3)c12)c1ccc(C(=O)O)cc1 ; C[C@H](NC(=O)c1cc(-c2cccc(C#N)c2)cc2[nH]cc(Sc3ccc(C(F)(F)F)cc3)c12)c1ccc(C(=O)O)cc1]

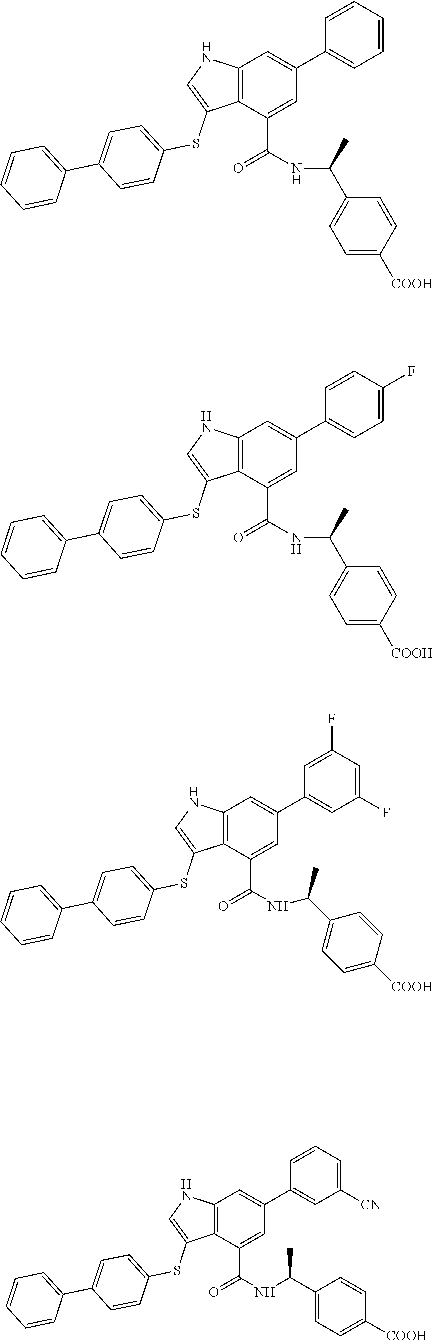 [SMILES: C[C@H](NC(=O)c1cc(-c2ccccc2)cc2[nH]cc(Sc3ccc(-c4ccccc4)cc3)c12)c1ccc(C(=O)O)cc1 ; C[C@H](NC(=O)c1cc(-c2ccc(F)cc2)cc2[nH]cc(Sc3ccc(-c4ccccc4)cc3)c12)c1ccc(C(=O)O)cc1 ; C[C@H](NC(=O)c1cc(-c2cc(F)cc(F)c2)cc2[nH]cc(Sc3ccc(-c4ccccc4)cc3)c12)c1ccc(C(=O)O)cc1 ; C[C@H](NC(=O)c1cc(-c2cccc(C#N)c2)cc2[nH]cc(Sc3ccc(-c4ccccc4)cc3)c12)c1ccc(C(=O)O)cc1]

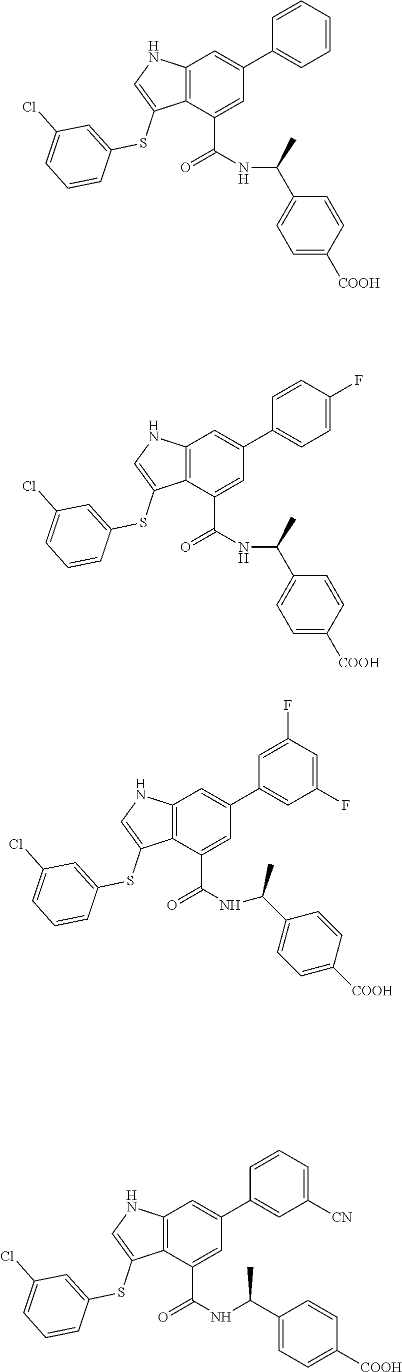 [SMILES: C[C@H](NC(=O)c1cc(-c2ccccc2)cc2[nH]cc(Sc3cccc(Cl)c3)c12)c1ccc(C(=O)O)cc1 ; C[C@H](NC(=O)c1cc(-c2ccc(F)cc2)cc2[nH]cc(Sc3cccc(Cl)c3)c12)c1ccc(C(=O)O)cc1 ; C[C@H](NC(=O)c1cc(-c2cc(F)cc(F)c2)cc2[nH]cc(Sc3cccc(Cl)c3)c12)c1ccc(C(=O)O)cc1 ; C[C@H](NC(=O)c1cc(-c2cccc(C#N)c2)cc2[nH]cc(Sc3cccc(Cl)c3)c12)c1ccc(C(=O)O)cc1]

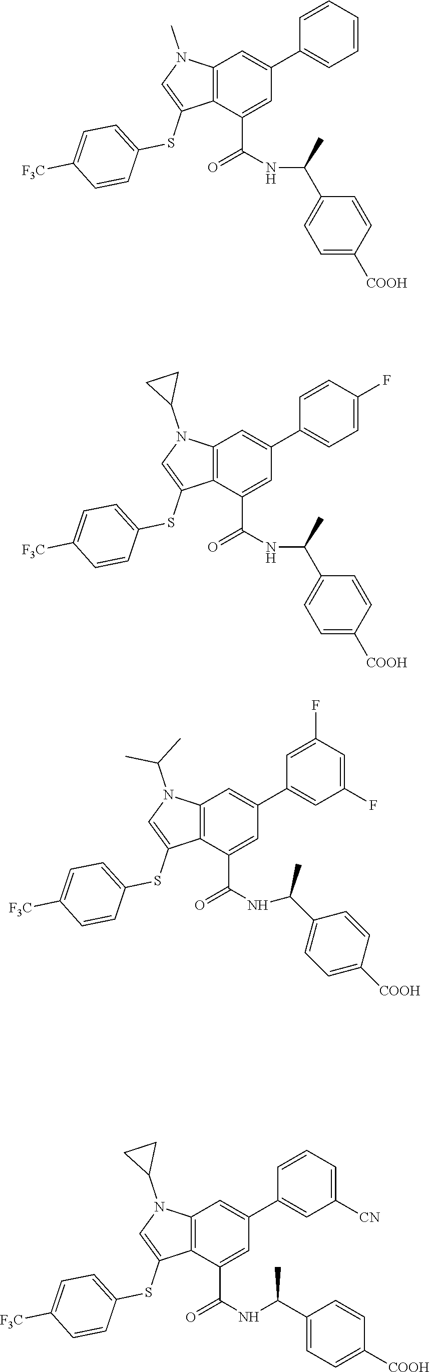 [SMILES: C[C@H](NC(=O)c1cc(-c2ccccc2)cc2c1c(Sc1ccc(C(F)(F)F)cc1)cn2C)c1ccc(C(=O)O)cc1 ; C[C@H](NC(=O)c1cc(-c2ccc(F)cc2)cc2c1c(Sc1ccc(C(F)(F)F)cc1)cn2C1CC1)c1ccc(C(=O)O)cc1 ; CC(C)n1cc(Sc2ccc(C(F)(F)F)cc2)c2c(C(=O)N[C@@H](C)c3ccc(C(=O)O)cc3)cc(-c3cc(F)cc(F)c3)cc21 ; C[C@H](NC(=O)c1cc(-c2cccc(C#N)c2)cc2c1c(Sc1ccc(C(F)(F)F)cc1)cn2C1CC1)c1ccc(C(=O)O)cc1]

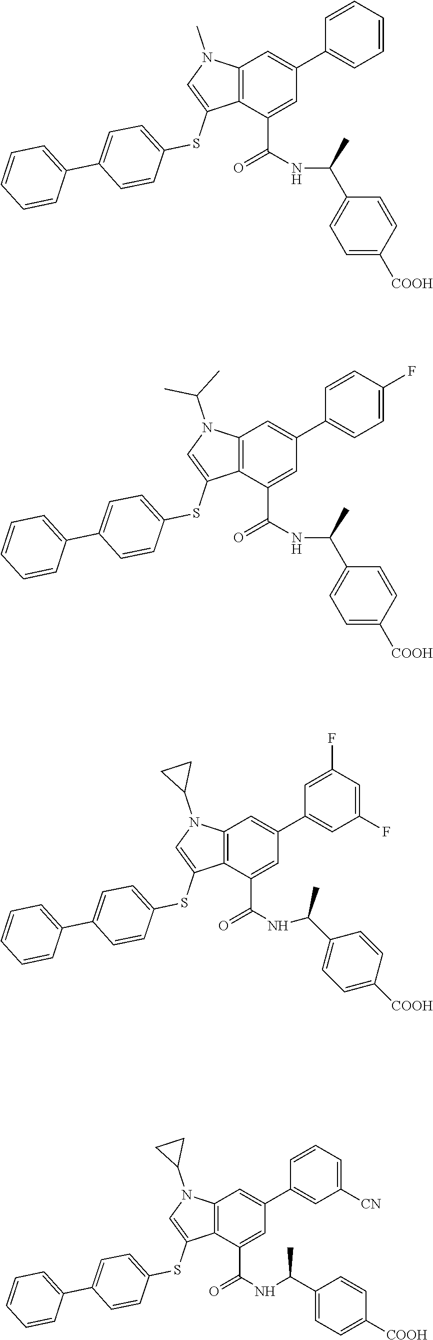 [SMILES: C[C@H](NC(=O)c1cc(-c2ccccc2)cc2c1c(Sc1ccc(-c3ccccc3)cc1)cn2C)c1ccc(C(=O)O)cc1 ; CC(C)n1cc(Sc2ccc(-c3ccccc3)cc2)c2c(C(=O)N[C@@H](C)c3ccc(C(=O)O)cc3)cc(-c3ccc(F)cc3)cc21 ; C[C@H](NC(=O)c1cc(-c2cc(F)cc(F)c2)cc2c1c(Sc1ccc(-c3ccccc3)cc1)cn2C1CC1)c1ccc(C(=O)O)cc1 ; C[C@H](NC(=O)c1cc(-c2cccc(C#N)c2)cc2c1c(Sc1ccc(-c3ccccc3)cc1)cn2C1CC1)c1ccc(C(=O)O)cc1]

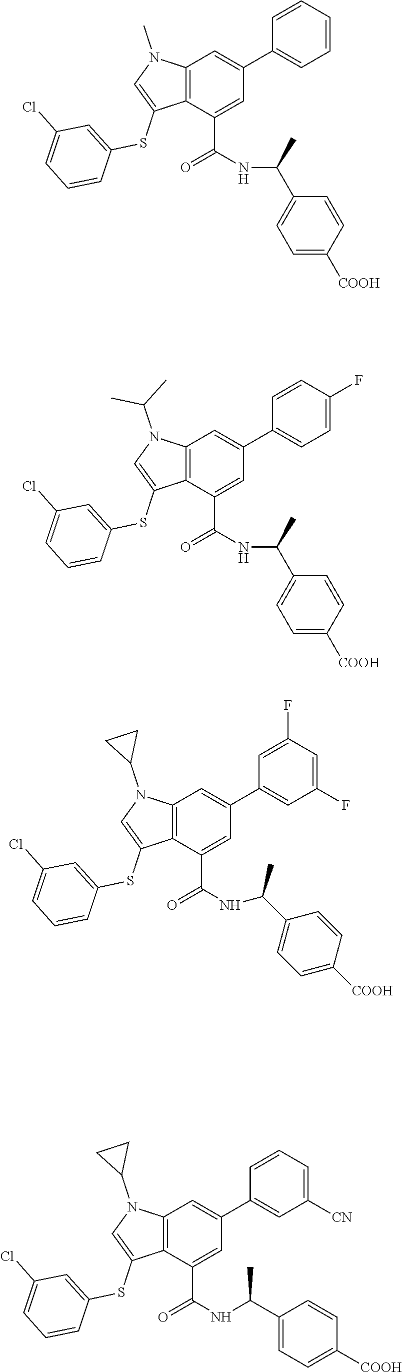 [SMILES: C[C@H](NC(=O)c1cc(-c2ccccc2)cc2c1c(Sc1cccc(Cl)c1)cn2C)c1ccc(C(=O)O)cc1 ; CC(C)n1cc(Sc2cccc(Cl)c2)c2c(C(=O)N[C@@H](C)c3ccc(C(=O)O)cc3)cc(-c3ccc(F)cc3)cc21 ; C[C@H](NC(=O)c1cc(-c2cc(F)cc(F)c2)cc2c1c(Sc1cccc(Cl)c1)cn2C1CC1)c1ccc(C(=O)O)cc1 ; C[C@H](NC(=O)c1cc(-c2cccc(C#N)c2)cc2c1c(Sc1cccc(Cl)c1)cn2C1CC1)c1ccc(C(=O)O)cc1]

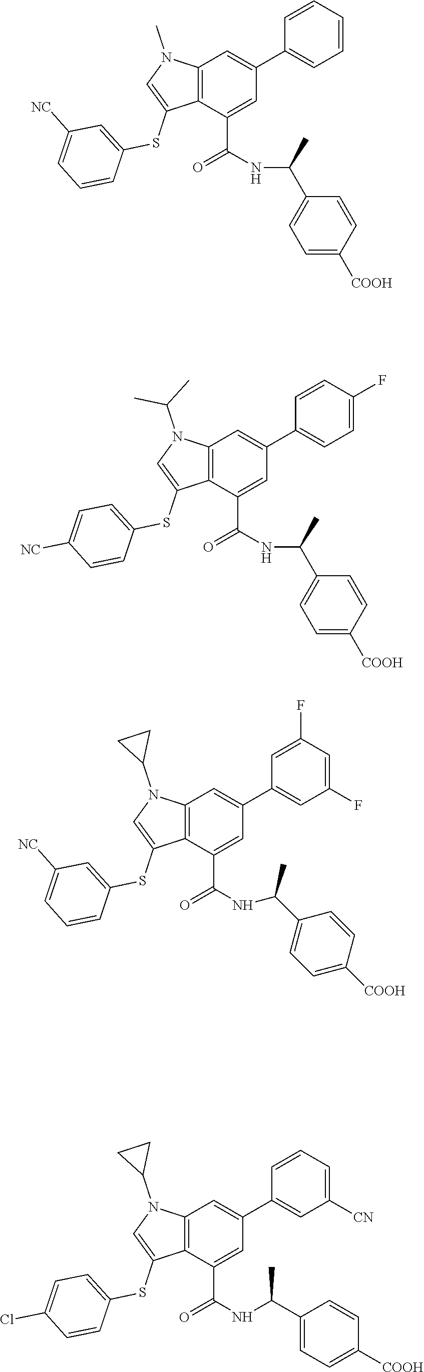 [SMILES: C[C@H](NC(=O)c1cc(-c2ccccc2)cc2c1c(Sc1cccc(C#N)c1)cn2C)c1ccc(C(=O)O)cc1 ; CC(C)n1cc(Sc2ccc(C#N)cc2)c2c(C(=O)N[C@@H](C)c3ccc(C(=O)O)cc3)cc(-c3ccc(F)cc3)cc21 ; C[C@H](NC(=O)c1cc(-c2cc(F)cc(F)c2)cc2c1c(Sc1cccc(C#N)c1)cn2C1CC1)c1ccc(C(=O)O)cc1 ; C[C@H](NC(=O)c1cc(-c2cccc(C#N)c2)cc2c1c(Sc1ccc(Cl)cc1)cn2C1CC1)c1ccc(C(=O)O)cc1]

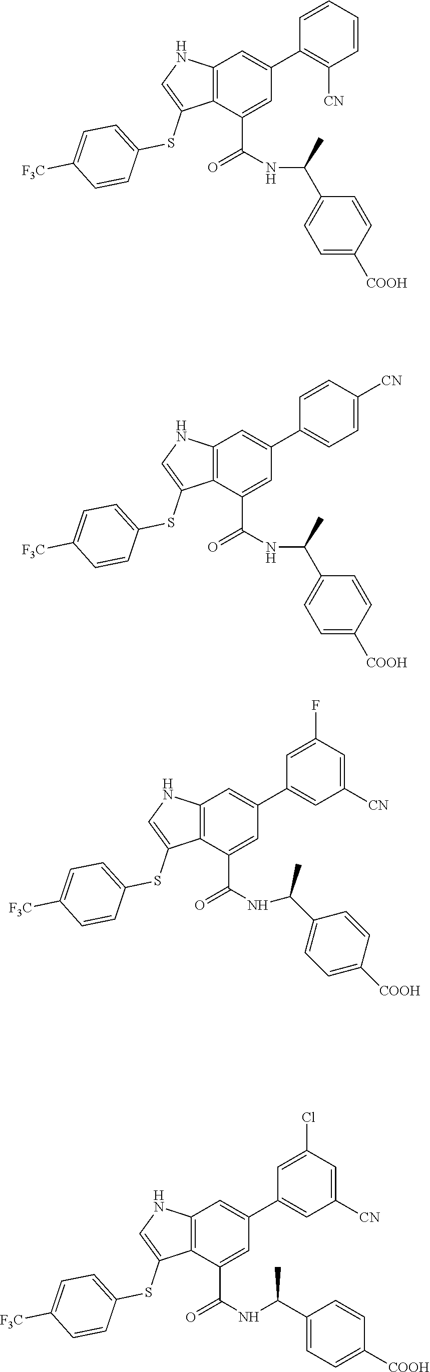 [SMILES: C[C@H](NC(=O)c1cc(-c2ccccc2C#N)cc2[nH]cc(Sc3ccc(C(F)(F)F)cc3)c12)c1ccc(C(=O)O)cc1 ; C[C@H](NC(=O)c1cc(-c2ccc(C#N)cc2)cc2[nH]cc(Sc3ccc(C(F)(F)F)cc3)c12)c1ccc(C(=O)O)cc1 ; C[C@H](NC(=O)c1cc(-c2cc(F)cc(C#N)c2)cc2[nH]cc(Sc3ccc(C(F)(F)F)cc3)c12)c1ccc(C(=O)O)cc1 ; C[C@H](NC(=O)c1cc(-c2cc(Cl)cc(C#N)c2)cc2[nH]cc(Sc3ccc(C(F)(F)F)cc3)c12)c1ccc(C(=O)O)cc1]

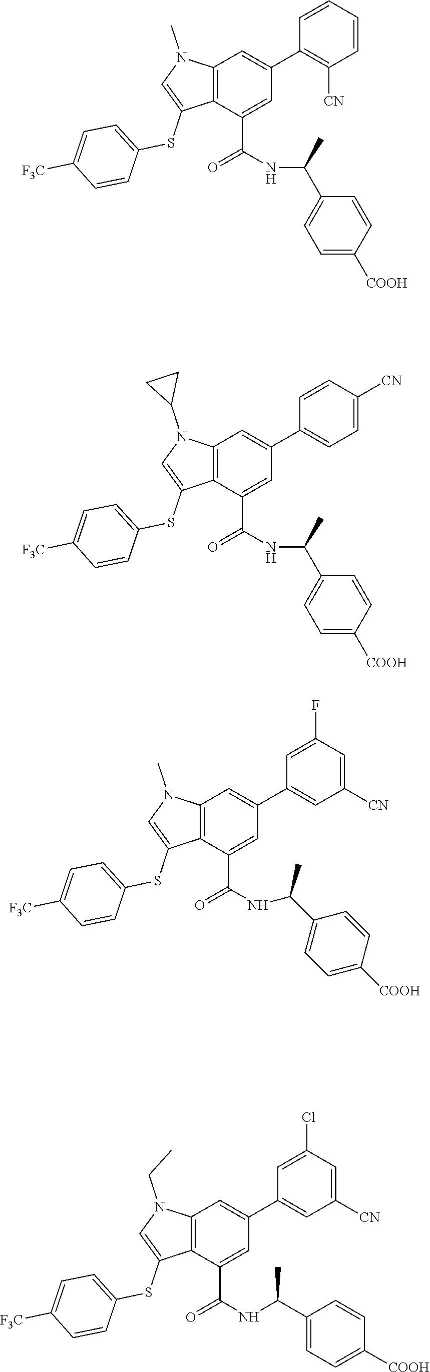 [SMILES: C[C@H](NC(=O)c1cc(-c2ccccc2C#N)cc2c1c(Sc1ccc(C(F)(F)F)cc1)cn2C)c1ccc(C(=O)O)cc1 ; C[C@H](NC(=O)c1cc(-c2ccc(C#N)cc2)cc2c1c(Sc1ccc(C(F)(F)F)cc1)cn2C1CC1)c1ccc(C(=O)O)cc1 ; C[C@H](NC(=O)c1cc(-c2cc(F)cc(C#N)c2)cc2c1c(Sc1ccc(C(F)(F)F)cc1)cn2C)c1ccc(C(=O)O)cc1 ; CCn1cc(Sc2ccc(C(F)(F)F)cc2)c2c(C(=O)N[C@@H](C)c3ccc(C(=O)O)cc3)cc(-c3cc(Cl)cc(C#N)c3)cc21]

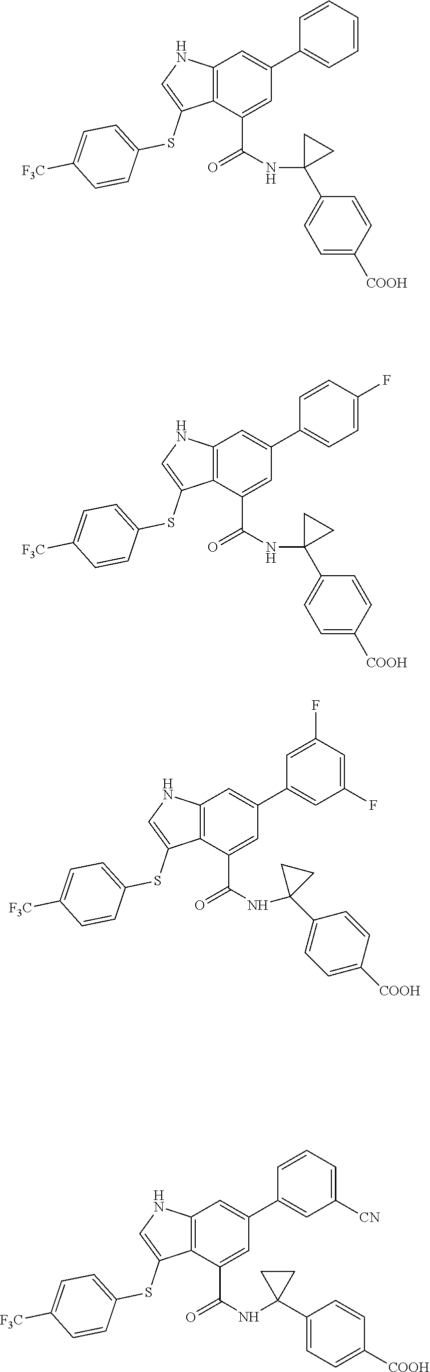 [SMILES: O=C(O)c1ccc(C2(NC(=O)c3cc(-c4ccccc4)cc4[nH]cc(Sc5ccc(C(F)(F)F)cc5)c34)CC2)cc1 ; O=C(O)c1ccc(C2(NC(=O)c3cc(-c4ccc(F)cc4)cc4[nH]cc(Sc5ccc(C(F)(F)F)cc5)c34)CC2)cc1 ; O=C(O)c1ccc(C2(NC(=O)c3cc(-c4cc(F)cc(F)c4)cc4[nH]cc(Sc5ccc(C(F)(F)F)cc5)c34)CC2)cc1 ; N#Cc1cccc(-c2cc(C(=O)NC3(c4ccc(C(=O)O)cc4)CC3)c3c(Sc4ccc(C(F)(F)F)cc4)c[nH]c3c2)c1]

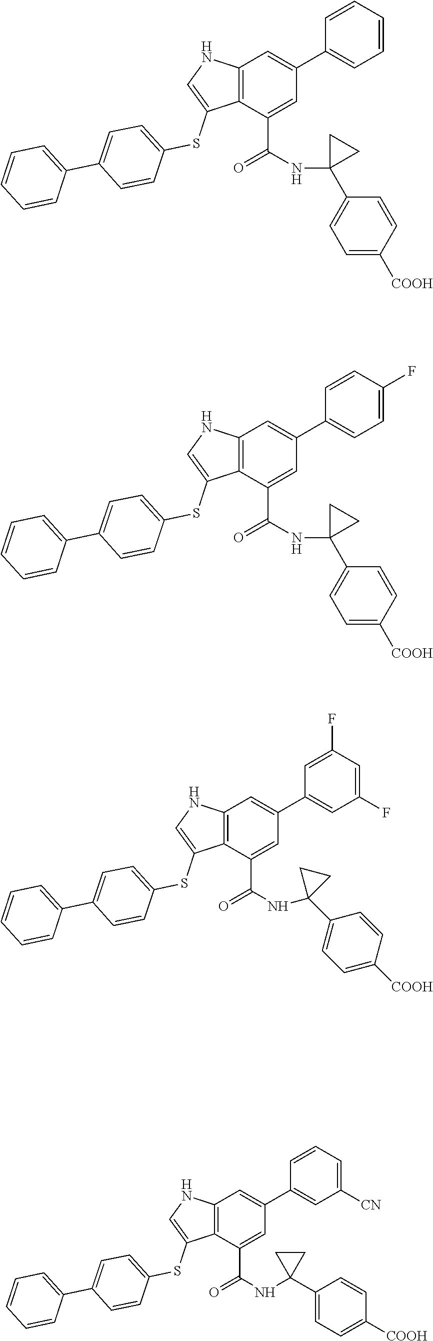 [SMILES: O=C(O)c1ccc(C2(NC(=O)c3cc(-c4ccccc4)cc4[nH]cc(Sc5ccc(-c6ccccc6)cc5)c34)CC2)cc1 ; O=C(O)c1ccc(C2(NC(=O)c3cc(-c4ccc(F)cc4)cc4[nH]cc(Sc5ccc(-c6ccccc6)cc5)c34)CC2)cc1 ; O=C(O)c1ccc(C2(NC(=O)c3cc(-c4cc(F)cc(F)c4)cc4[nH]cc(Sc5ccc(-c6ccccc6)cc5)c34)CC2)cc1 ; N#Cc1cccc(-c2cc(C(=O)NC3(c4ccc(C(=O)O)cc4)CC3)c3c(Sc4ccc(-c5ccccc5)cc4)c[nH]c3c2)c1]

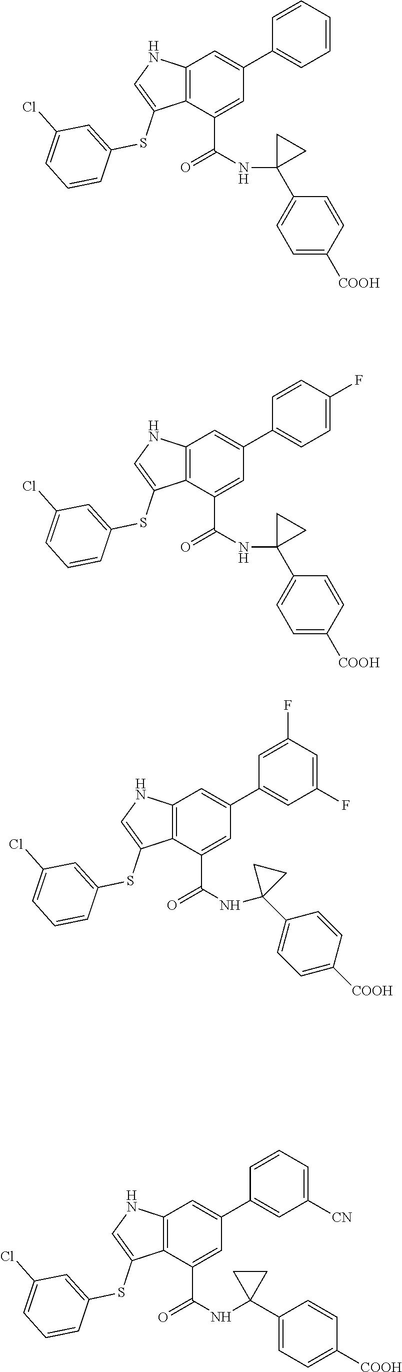 [SMILES: O=C(O)c1ccc(C2(NC(=O)c3cc(-c4ccccc4)cc4[nH]cc(Sc5cccc(Cl)c5)c34)CC2)cc1 ; O=C(O)c1ccc(C2(NC(=O)c3cc(-c4ccc(F)cc4)cc4[nH]cc(Sc5cccc(Cl)c5)c34)CC2)cc1 ; O=C(O)c1ccc(C2(NC(=O)c3cc(-c4cc(F)cc(F)c4)cc4[nH]cc(Sc5cccc(Cl)c5)c34)CC2)cc1 ; N#Cc1cccc(-c2cc(C(=O)NC3(c4ccc(C(=O)O)cc4)CC3)c3c(Sc4cccc(Cl)c4)c[nH]c3c2)c1]

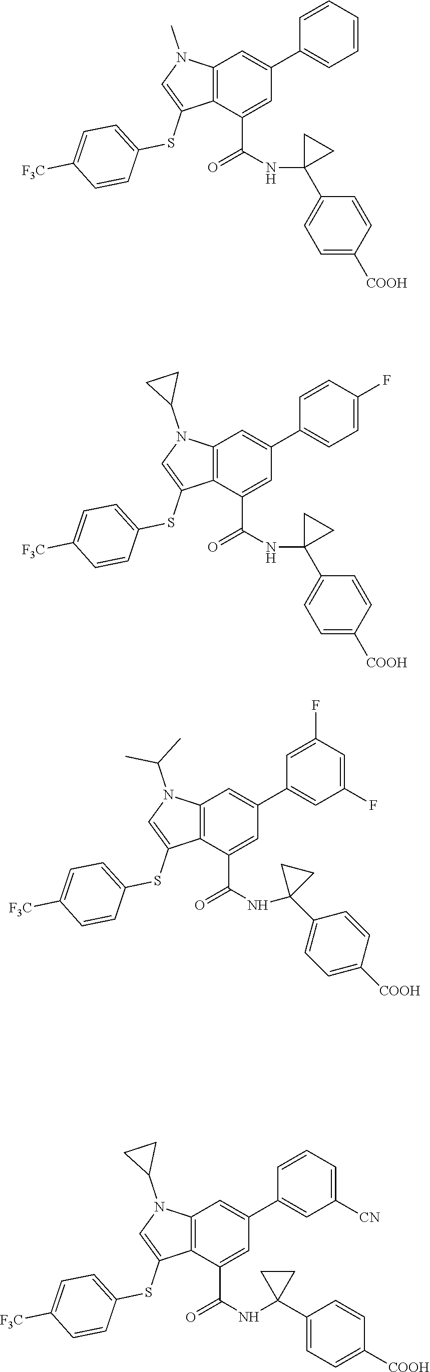 [SMILES: Cn1cc(Sc2ccc(C(F)(F)F)cc2)c2c(C(=O)NC3(c4ccc(C(=O)O)cc4)CC3)cc(-c3ccccc3)cc21 ; O=C(O)c1ccc(C2(NC(=O)c3cc(-c4ccc(F)cc4)cc4c3c(Sc3ccc(C(F)(F)F)cc3)cn4C3CC3)CC2)cc1 ; CC(C)n1cc(Sc2ccc(C(F)(F)F)cc2)c2c(C(=O)NC3(c4ccc(C(=O)O)cc4)CC3)cc(-c3cc(F)cc(F)c3)cc21 ; N#Cc1cccc(-c2cc(C(=O)NC3(c4ccc(C(=O)O)cc4)CC3)c3c(Sc4ccc(C(F)(F)F)cc4)cn(C4CC4)c3c2)c1]

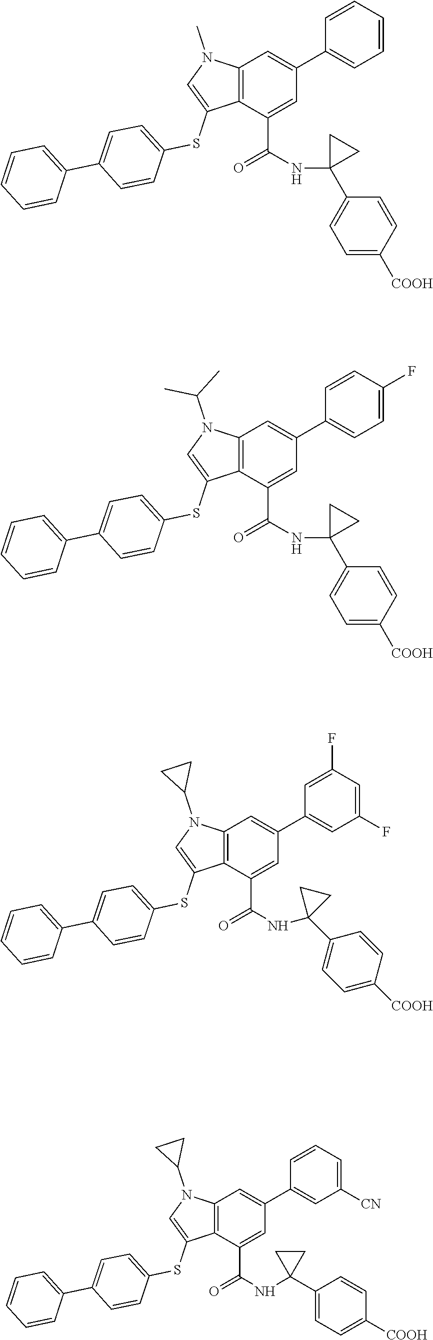 [SMILES: Cn1cc(Sc2ccc(-c3ccccc3)cc2)c2c(C(=O)NC3(c4ccc(C(=O)O)cc4)CC3)cc(-c3ccccc3)cc21 ; CC(C)n1cc(Sc2ccc(-c3ccccc3)cc2)c2c(C(=O)NC3(c4ccc(C(=O)O)cc4)CC3)cc(-c3ccc(F)cc3)cc21 ; O=C(O)c1ccc(C2(NC(=O)c3cc(-c4cc(F)cc(F)c4)cc4c3c(Sc3ccc(-c5ccccc5)cc3)cn4C3CC3)CC2)cc1 ; N#Cc1cccc(-c2cc(C(=O)NC3(c4ccc(C(=O)O)cc4)CC3)c3c(Sc4ccc(-c5ccccc5)cc4)cn(C4CC4)c3c2)c1]

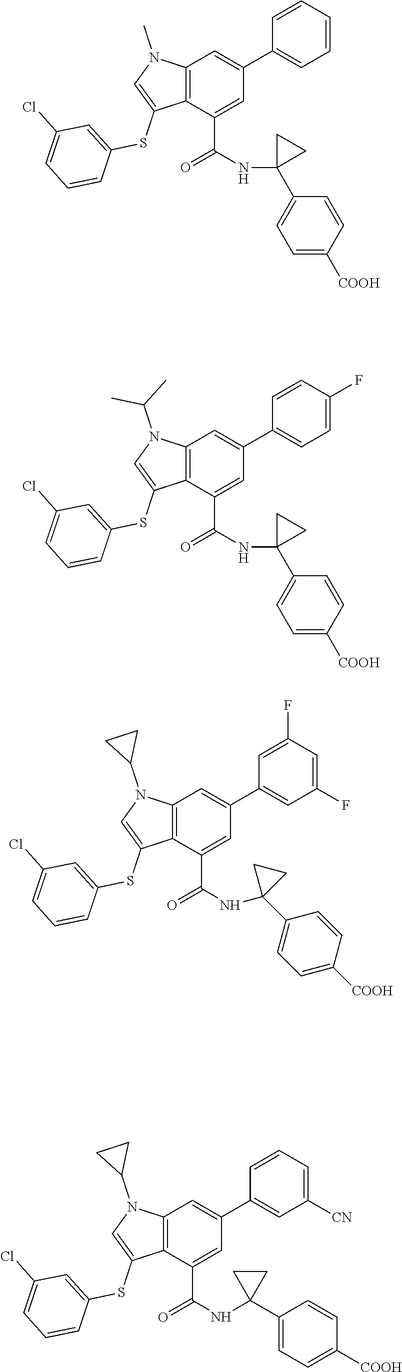 [SMILES: Cn1cc(Sc2cccc(Cl)c2)c2c(C(=O)NC3(c4ccc(C(=O)O)cc4)CC3)cc(-c3ccccc3)cc21 ; CC(C)n1cc(Sc2cccc(Cl)c2)c2c(C(=O)NC3(c4ccc(C(=O)O)cc4)CC3)cc(-c3ccc(F)cc3)cc21 ; O=C(O)c1ccc(C2(NC(=O)c3cc(-c4cc(F)cc(F)c4)cc4c3c(Sc3cccc(Cl)c3)cn4C3CC3)CC2)cc1 ; N#Cc1cccc(-c2cc(C(=O)NC3(c4ccc(C(=O)O)cc4)CC3)c3c(Sc4cccc(Cl)c4)cn(C4CC4)c3c2)c1]

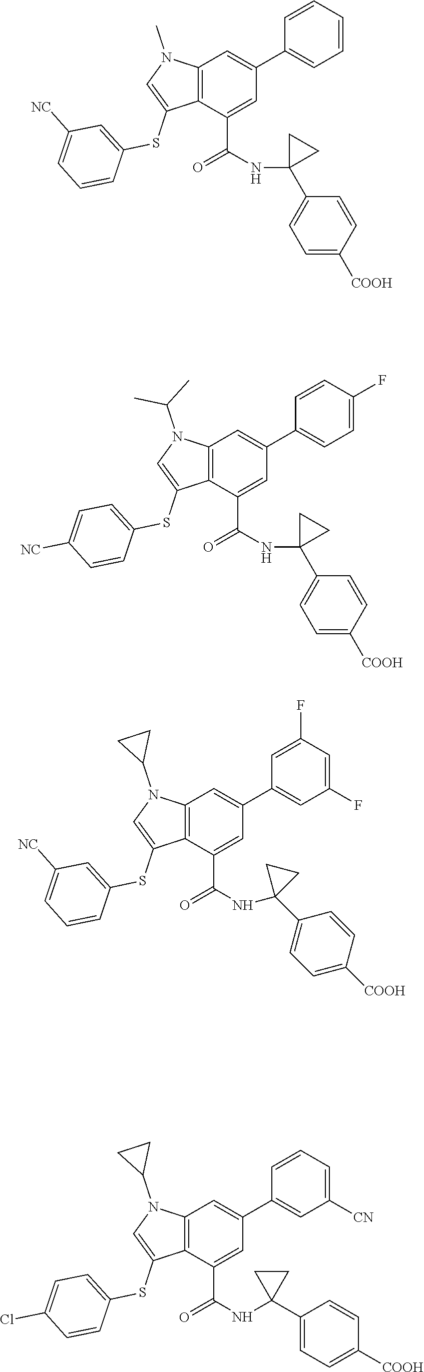 [SMILES: Cn1cc(Sc2cccc(C#N)c2)c2c(C(=O)NC3(c4ccc(C(=O)O)cc4)CC3)cc(-c3ccccc3)cc21 ; CC(C)n1cc(Sc2ccc(C#N)cc2)c2c(C(=O)NC3(c4ccc(C(=O)O)cc4)CC3)cc(-c3ccc(F)cc3)cc21 ; N#Cc1cccc(Sc2cn(C3CC3)c3cc(-c4cc(F)cc(F)c4)cc(C(=O)NC4(c5ccc(C(=O)O)cc5)CC4)c23)c1 ; N#Cc1cccc(-c2cc(C(=O)NC3(c4ccc(C(=O)O)cc4)CC3)c3c(Sc4ccc(Cl)cc4)cn(C4CC4)c3c2)c1]

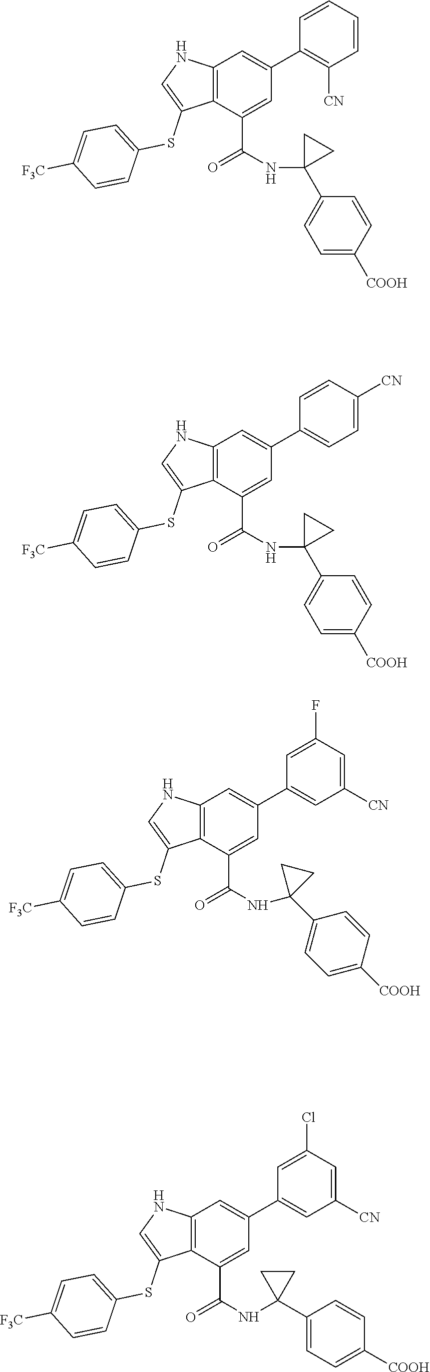 [SMILES: N#Cc1ccccc1-c1cc(C(=O)NC2(c3ccc(C(=O)O)cc3)CC2)c2c(Sc3ccc(C(F)(F)F)cc3)c[nH]c2c1 ; N#Cc1ccc(-c2cc(C(=O)NC3(c4ccc(C(=O)O)cc4)CC3)c3c(Sc4ccc(C(F)(F)F)cc4)c[nH]c3c2)cc1 ; N#Cc1cc(F)cc(-c2cc(C(=O)NC3(c4ccc(C(=O)O)cc4)CC3)c3c(Sc4ccc(C(F)(F)F)cc4)c[nH]c3c2)c1 ; N#Cc1cc(Cl)cc(-c2cc(C(=O)NC3(c4ccc(C(=O)O)cc4)CC3)c3c(Sc4ccc(C(F)(F)F)cc4)c[nH]c3c2)c1]

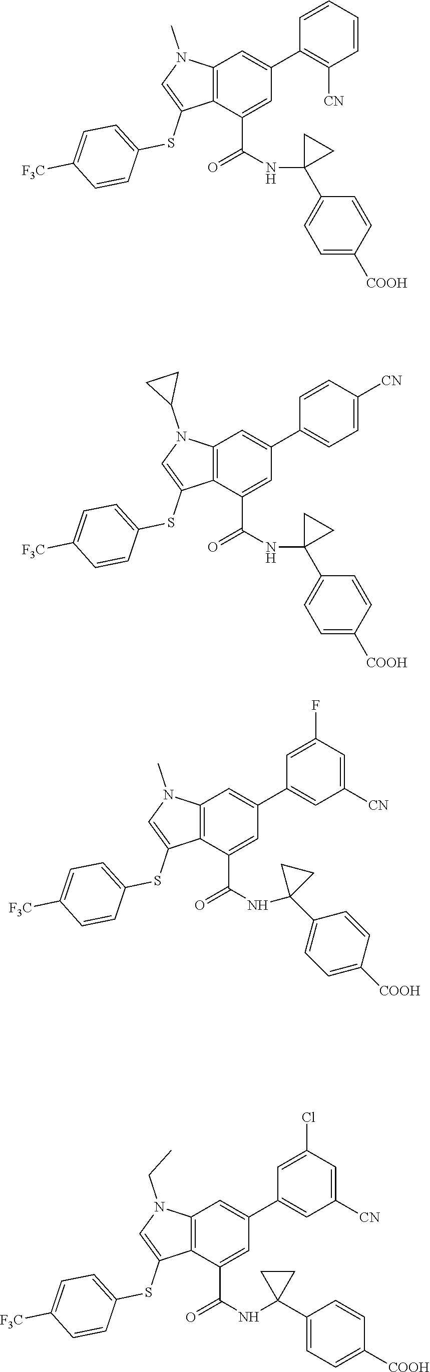 [SMILES: Cn1cc(Sc2ccc(C(F)(F)F)cc2)c2c(C(=O)NC3(c4ccc(C(=O)O)cc4)CC3)cc(-c3ccccc3C#N)cc21 ; N#Cc1ccc(-c2cc(C(=O)NC3(c4ccc(C(=O)O)cc4)CC3)c3c(Sc4ccc(C(F)(F)F)cc4)cn(C4CC4)c3c2)cc1 ; Cn1cc(Sc2ccc(C(F)(F)F)cc2)c2c(C(=O)NC3(c4ccc(C(=O)O)cc4)CC3)cc(-c3cc(F)cc(C#N)c3)cc21 ; CCn1cc(Sc2ccc(C(F)(F)F)cc2)c2c(C(=O)NC3(c4ccc(C(=O)O)cc4)CC3)cc(-c3cc(Cl)cc(C#N)c3)cc21]

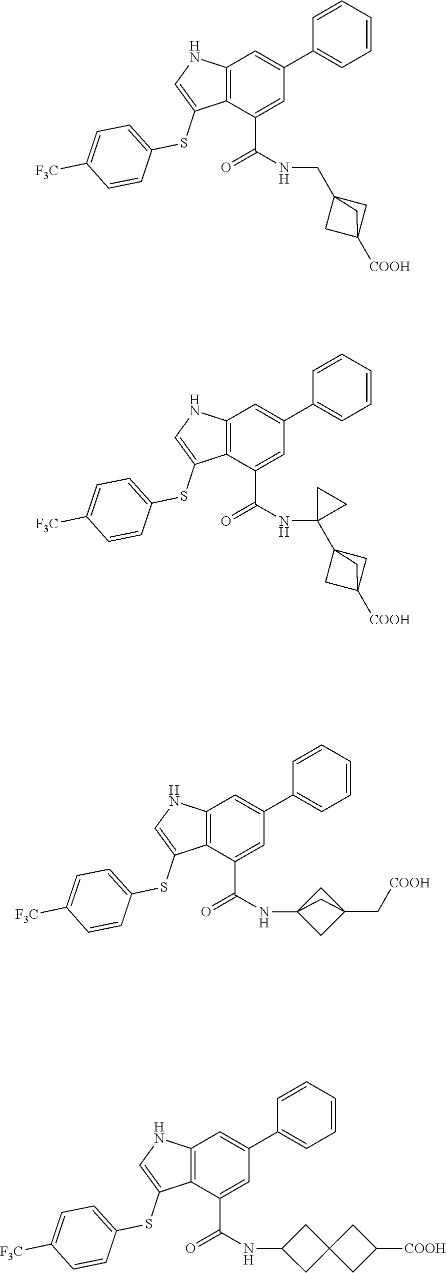 [SMILES: O=C(NCC12CC(C(=O)O)(C1)C2)c1cc(-c2ccccc2)cc2[nH]cc(Sc3ccc(C(F)(F)F)cc3)c12 ; O=C(NC1(C23CC(C(=O)O)(C2)C3)CC1)c1cc(-c2ccccc2)cc2[nH]cc(Sc3ccc(C(F)(F)F)cc3)c12 ; O=C(O)CC12CC(NC(=O)c3cc(-c4ccccc4)cc4[nH]cc(Sc5ccc(C(F)(F)F)cc5)c34)(C1)C2 ; O=C(NC1CC2(C1)CC(C(=O)O)C2)c1cc(-c2ccccc2)cc2[nH]cc(Sc3ccc(C(F)(F)F)cc3)c12]

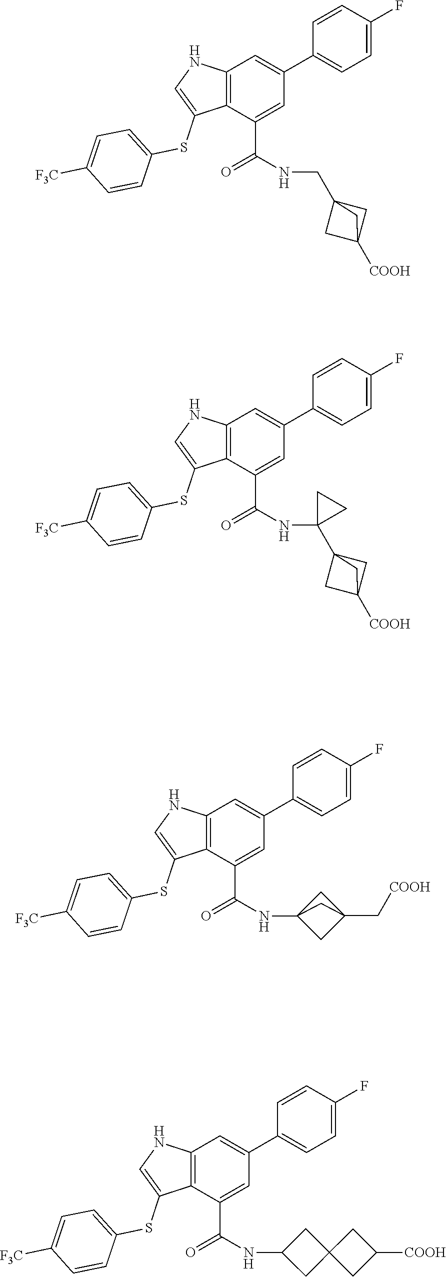 [SMILES: O=C(NCC12CC(C(=O)O)(C1)C2)c1cc(-c2ccc(F)cc2)cc2[nH]cc(Sc3ccc(C(F)(F)F)cc3)c12 ; O=C(NC1(C23CC(C(=O)O)(C2)C3)CC1)c1cc(-c2ccc(F)cc2)cc2[nH]cc(Sc3ccc(C(F)(F)F)cc3)c12 ; O=C(O)CC12CC(NC(=O)c3cc(-c4ccc(F)cc4)cc4[nH]cc(Sc5ccc(C(F)(F)F)cc5)c34)(C1)C2 ; O=C(NC1CC2(C1)CC(C(=O)O)C2)c1cc(-c2ccc(F)cc2)cc2[nH]cc(Sc3ccc(C(F)(F)F)cc3)c12]

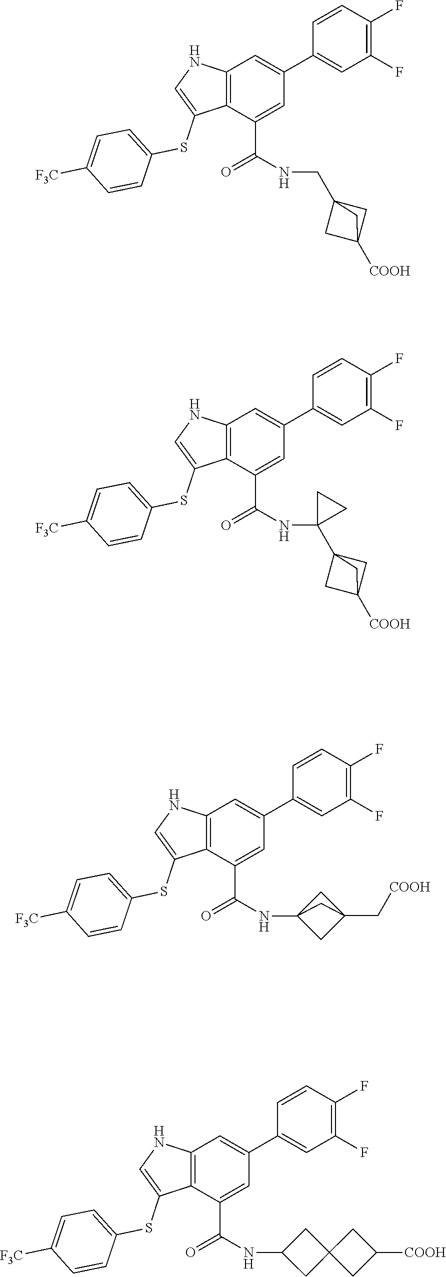 [SMILES: O=C(NCC12CC(C(=O)O)(C1)C2)c1cc(-c2ccc(F)c(F)c2)cc2[nH]cc(Sc3ccc(C(F)(F)F)cc3)c12 ; O=C(NC1(C23CC(C(=O)O)(C2)C3)CC1)c1cc(-c2ccc(F)c(F)c2)cc2[nH]cc(Sc3ccc(C(F)(F)F)cc3)c12 ; O=C(O)CC12CC(NC(=O)c3cc(-c4ccc(F)c(F)c4)cc4[nH]cc(Sc5ccc(C(F)(F)F)cc5)c34)(C1)C2 ; O=C(NC1CC2(C1)CC(C(=O)O)C2)c1cc(-c2ccc(F)c(F)c2)cc2[nH]cc(Sc3ccc(C(F)(F)F)cc3)c12]

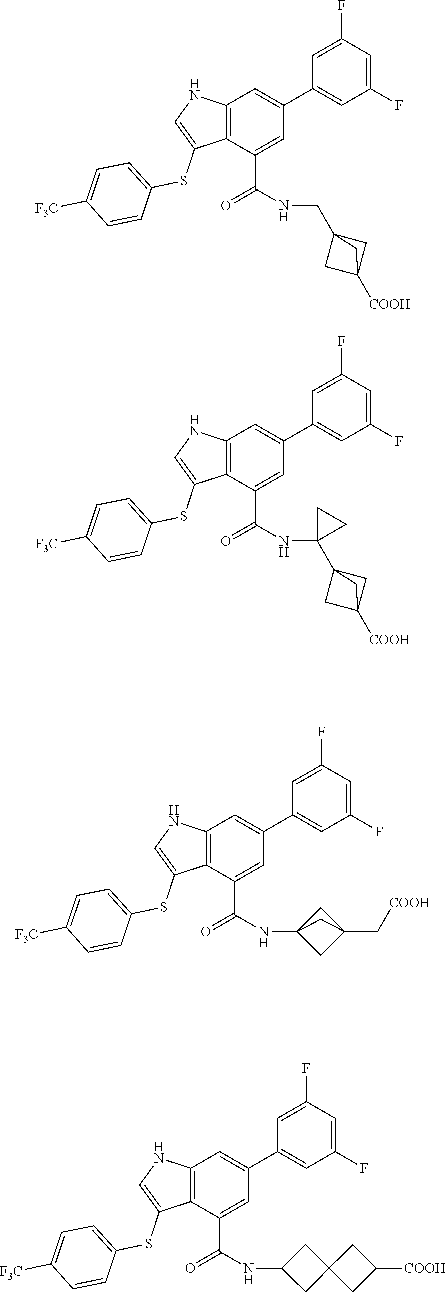 [SMILES: O=C(NCC12CC(C(=O)O)(C1)C2)c1cc(-c2cc(F)cc(F)c2)cc2[nH]cc(Sc3ccc(C(F)(F)F)cc3)c12 ; O=C(NC1(C23CC(C(=O)O)(C2)C3)CC1)c1cc(-c2cc(F)cc(F)c2)cc2[nH]cc(Sc3ccc(C(F)(F)F)cc3)c12 ; O=C(O)CC12CC(NC(=O)c3cc(-c4cc(F)cc(F)c4)cc4[nH]cc(Sc5ccc(C(F)(F)F)cc5)c34)(C1)C2 ; O=C(NC1CC2(C1)CC(C(=O)O)C2)c1cc(-c2cc(F)cc(F)c2)cc2[nH]cc(Sc3ccc(C(F)(F)F)cc3)c12]

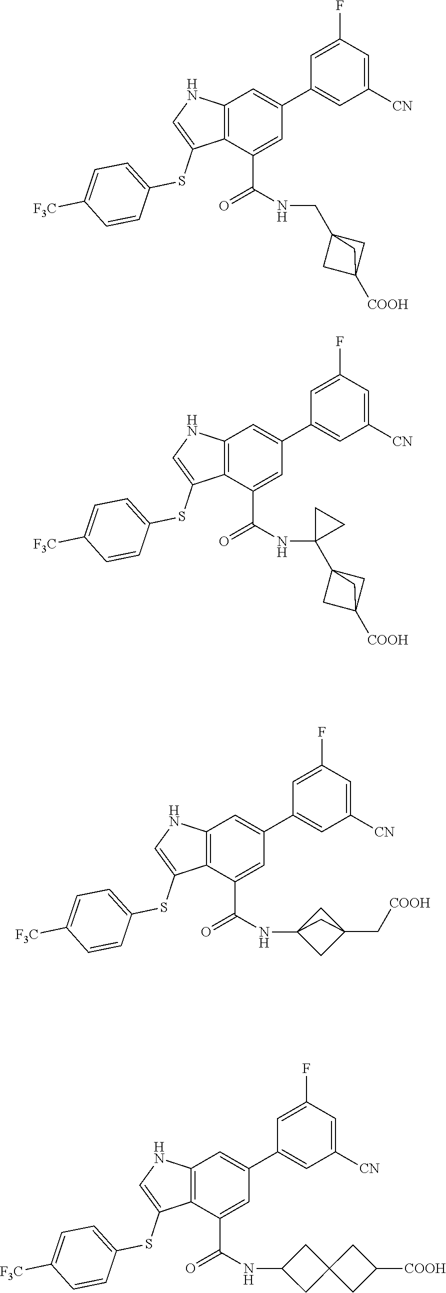 [SMILES: N#Cc1cc(F)cc(-c2cc(C(=O)NCC34CC(C(=O)O)(C3)C4)c3c(Sc4ccc(C(F)(F)F)cc4)c[nH]c3c2)c1 ; N#Cc1cc(F)cc(-c2cc(C(=O)NC3(C45CC(C(=O)O)(C4)C5)CC3)c3c(Sc4ccc(C(F)(F)F)cc4)c[nH]c3c2)c1 ; N#Cc1cc(F)cc(-c2cc(C(=O)NC34CC(CC(=O)O)(C3)C4)c3c(Sc4ccc(C(F)(F)F)cc4)c[nH]c3c2)c1 ; N#Cc1cc(F)cc(-c2cc(C(=O)NC3CC4(C3)CC(C(=O)O)C4)c3c(Sc4ccc(C(F)(F)F)cc4)c[nH]c3c2)c1]

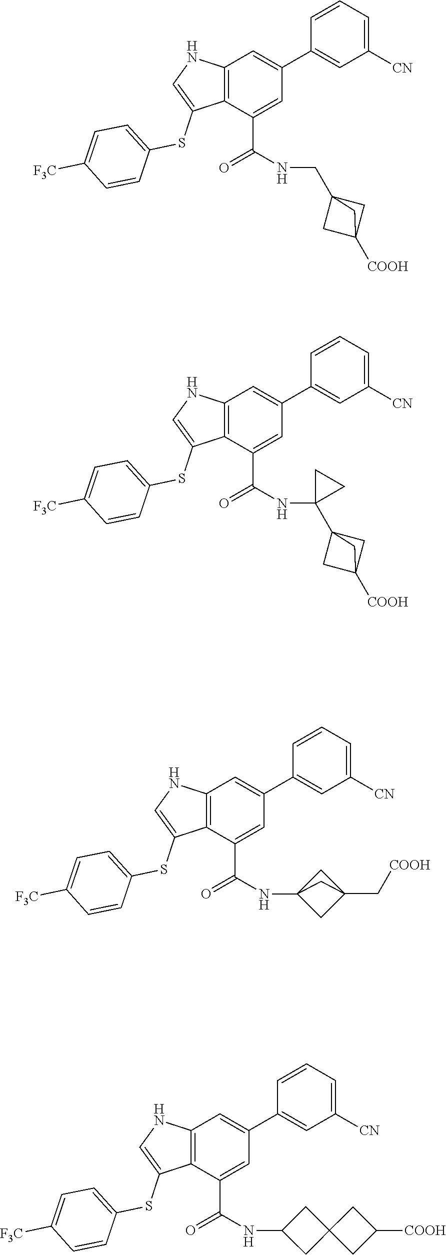 [SMILES: N#Cc1cccc(-c2cc(C(=O)NCC34CC(C(=O)O)(C3)C4)c3c(Sc4ccc(C(F)(F)F)cc4)c[nH]c3c2)c1 ; N#Cc1cccc(-c2cc(C(=O)NC3(C45CC(C(=O)O)(C4)C5)CC3)c3c(Sc4ccc(C(F)(F)F)cc4)c[nH]c3c2)c1 ; N#Cc1cccc(-c2cc(C(=O)NC34CC(CC(=O)O)(C3)C4)c3c(Sc4ccc(C(F)(F)F)cc4)c[nH]c3c2)c1 ; N#Cc1cccc(-c2cc(C(=O)NC3CC4(C3)CC(C(=O)O)C4)c3c(Sc4ccc(C(F)(F)F)cc4)c[nH]c3c2)c1]

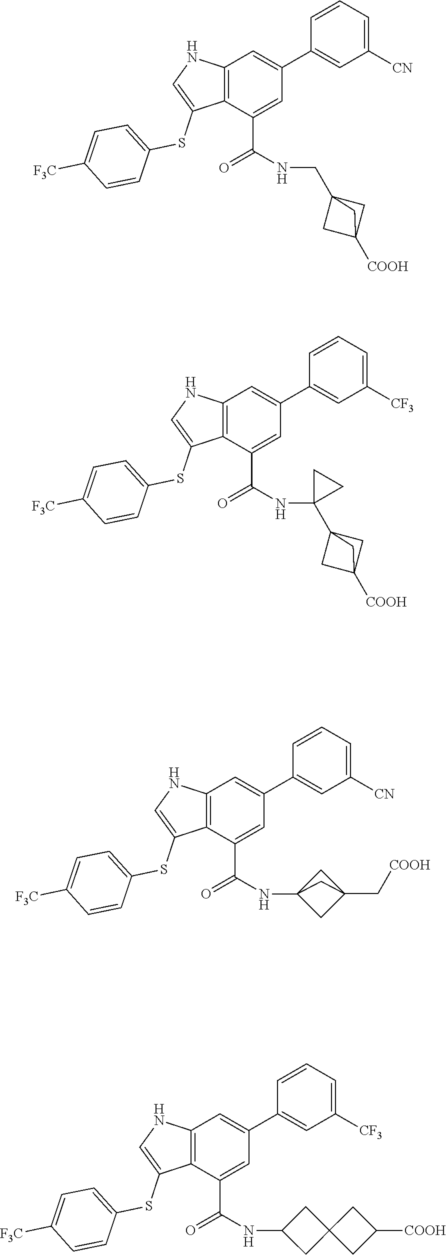 [SMILES: N#Cc1cccc(-c2cc(C(=O)NCC34CC(C(=O)O)(C3)C4)c3c(Sc4ccc(C(F)(F)F)cc4)c[nH]c3c2)c1 ; O=C(NC1(C23CC(C(=O)O)(C2)C3)CC1)c1cc(-c2cccc(C(F)(F)F)c2)cc2[nH]cc(Sc3ccc(C(F)(F)F)cc3)c12 ; N#Cc1cccc(-c2cc(C(=O)NC34CC(CC(=O)O)(C3)C4)c3c(Sc4ccc(C(F)(F)F)cc4)c[nH]c3c2)c1 ; O=C(NC1CC2(C1)CC(C(=O)O)C2)c1cc(-c2cccc(C(F)(F)F)c2)cc2[nH]cc(Sc3ccc(C(F)(F)F)cc3)c12]

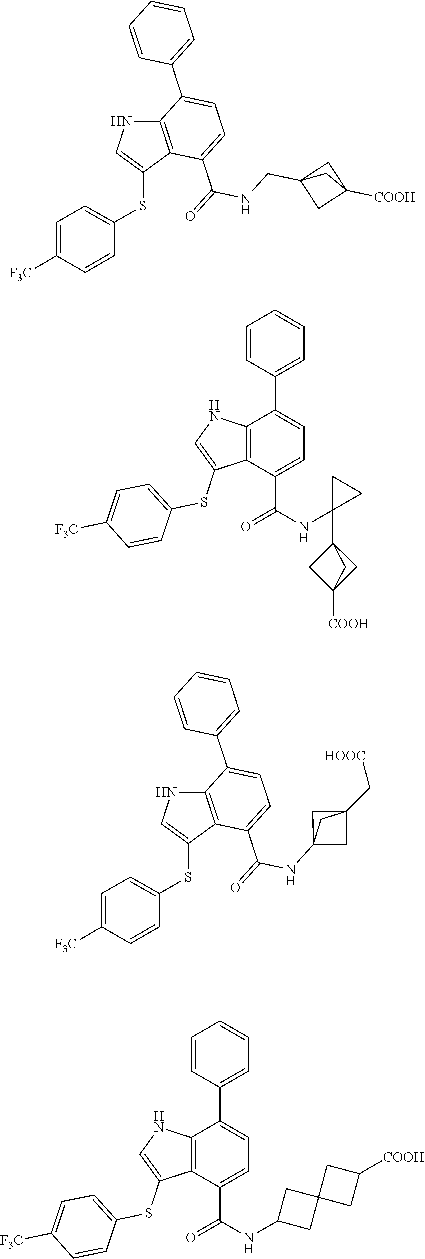 [SMILES: O=C(NCC12CC(C(=O)O)(C1)C2)c1ccc(-c2ccccc2)c2[nH]cc(Sc3ccc(C(F)(F)F)cc3)c12 ; O=C(NC1(C23CC(C(=O)O)(C2)C3)CC1)c1ccc(-c2ccccc2)c2[nH]cc(Sc3ccc(C(F)(F)F)cc3)c12 ; O=C(O)CC12CC(NC(=O)c3ccc(-c4ccccc4)c4[nH]cc(Sc5ccc(C(F)(F)F)cc5)c34)(C1)C2 ; O=C(NC1CC2(C1)CC(C(=O)O)C2)c1ccc(-c2ccccc2)c2[nH]cc(Sc3ccc(C(F)(F)F)cc3)c12]

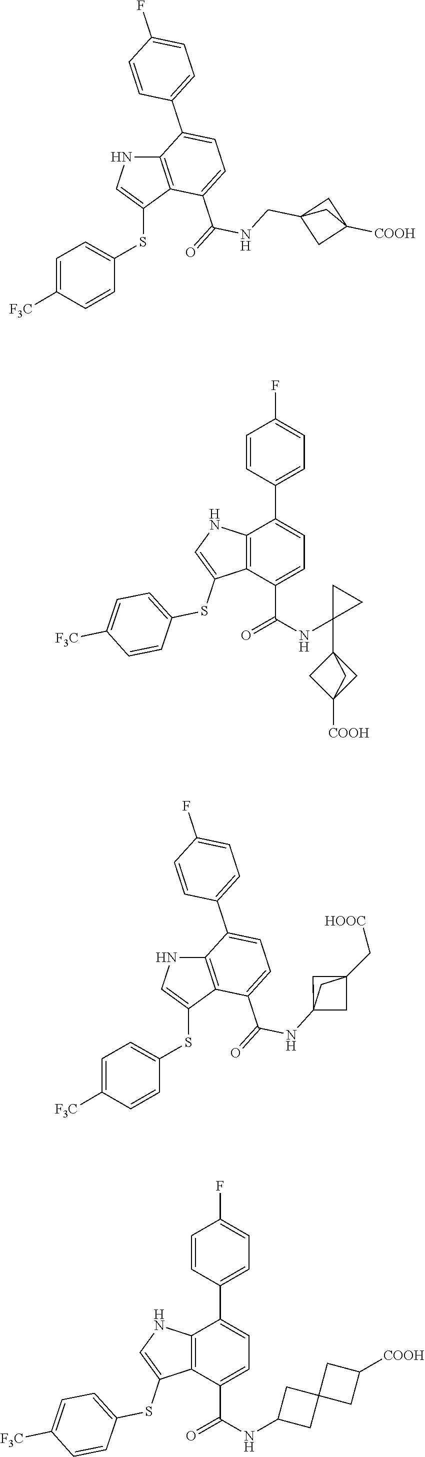 [SMILES: O=C(NCC12CC(C(=O)O)(C1)C2)c1ccc(-c2ccc(F)cc2)c2[nH]cc(Sc3ccc(C(F)(F)F)cc3)c12 ; O=C(NC1(C23CC(C(=O)O)(C2)C3)CC1)c1ccc(-c2ccc(F)cc2)c2[nH]cc(Sc3ccc(C(F)(F)F)cc3)c12 ; O=C(O)CC12CC(NC(=O)c3ccc(-c4ccc(F)cc4)c4[nH]cc(Sc5ccc(C(F)(F)F)cc5)c34)(C1)C2 ; O=C(NC1CC2(C1)CC(C(=O)O)C2)c1ccc(-c2ccc(F)cc2)c2[nH]cc(Sc3ccc(C(F)(F)F)cc3)c12]

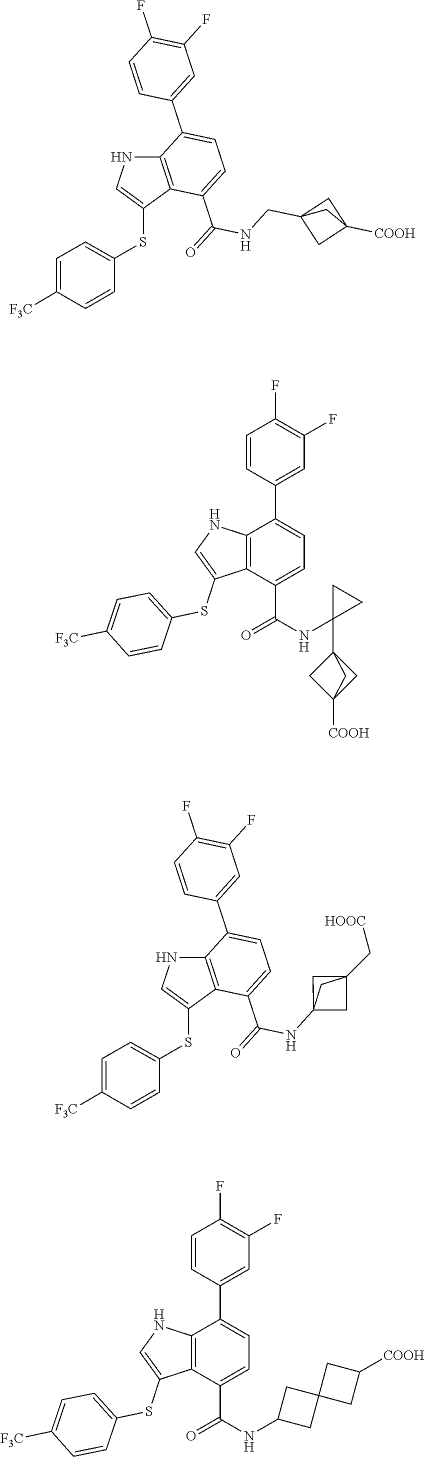 [SMILES: O=C(NCC12CC(C(=O)O)(C1)C2)c1ccc(-c2ccc(F)c(F)c2)c2[nH]cc(Sc3ccc(C(F)(F)F)cc3)c12 ; O=C(NC1(C23CC(C(=O)O)(C2)C3)CC1)c1ccc(-c2ccc(F)c(F)c2)c2[nH]cc(Sc3ccc(C(F)(F)F)cc3)c12 ; O=C(O)CC12CC(NC(=O)c3ccc(-c4ccc(F)c(F)c4)c4[nH]cc(Sc5ccc(C(F)(F)F)cc5)c34)(C1)C2 ; O=C(NC1CC2(C1)CC(C(=O)O)C2)c1ccc(-c2ccc(F)c(F)c2)c2[nH]cc(Sc3ccc(C(F)(F)F)cc3)c12]

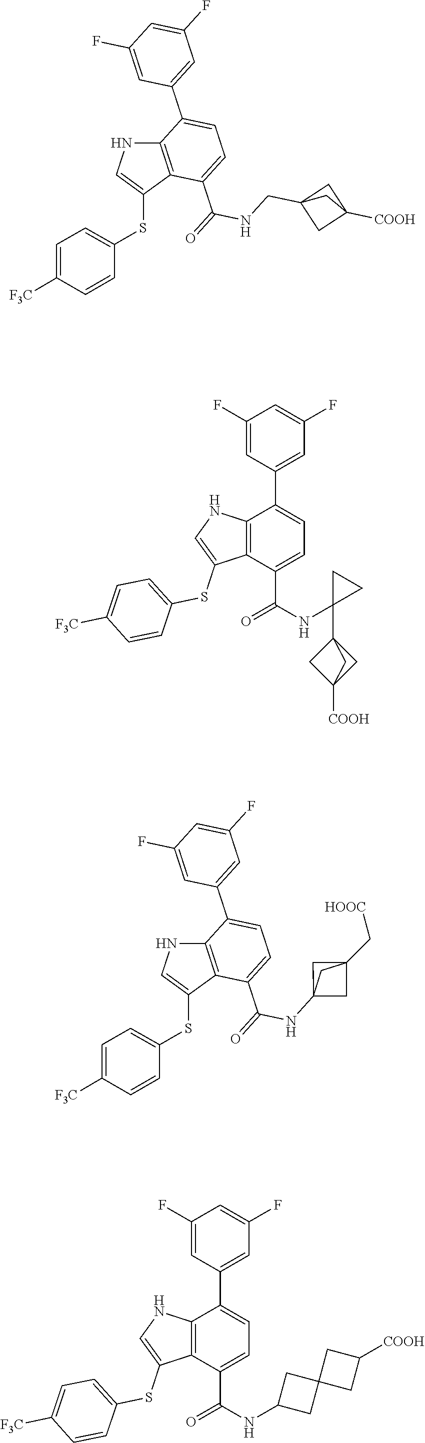 [SMILES: O=C(NCC12CC(C(=O)O)(C1)C2)c1ccc(-c2cc(F)cc(F)c2)c2[nH]cc(Sc3ccc(C(F)(F)F)cc3)c12 ; O=C(NC1(C23CC(C(=O)O)(C2)C3)CC1)c1ccc(-c2cc(F)cc(F)c2)c2[nH]cc(Sc3ccc(C(F)(F)F)cc3)c12 ; O=C(O)CC12CC(NC(=O)c3ccc(-c4cc(F)cc(F)c4)c4[nH]cc(Sc5ccc(C(F)(F)F)cc5)c34)(C1)C2 ; O=C(NC1CC2(C1)CC(C(=O)O)C2)c1ccc(-c2cc(F)cc(F)c2)c2[nH]cc(Sc3ccc(C(F)(F)F)cc3)c12]

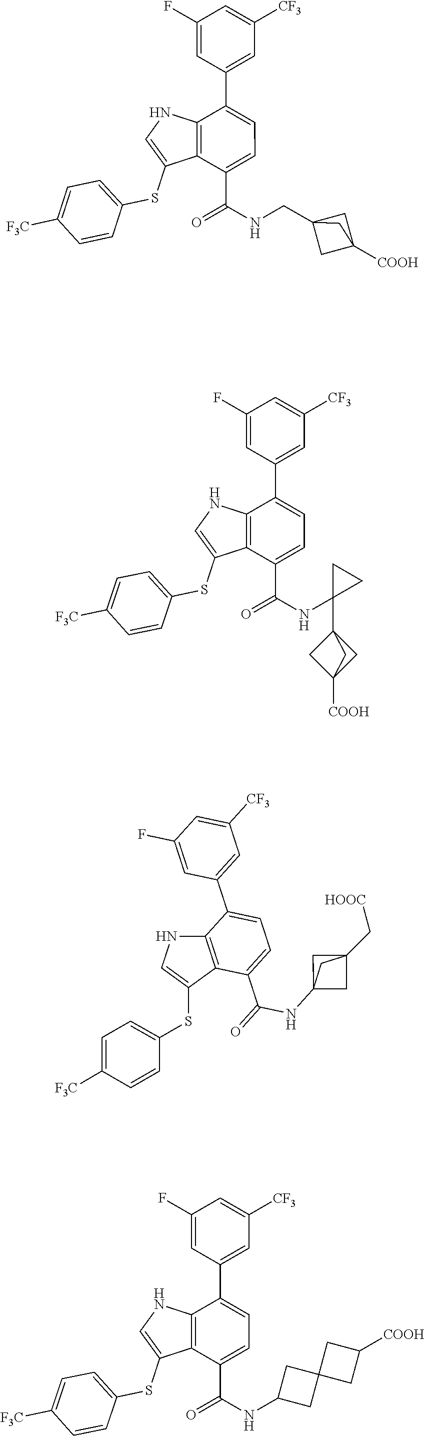 [SMILES: O=C(NCC12CC(C(=O)O)(C1)C2)c1ccc(-c2cc(F)cc(C(F)(F)F)c2)c2[nH]cc(Sc3ccc(C(F)(F)F)cc3)c12 ; O=C(NC1(C23CC(C(=O)O)(C2)C3)CC1)c1ccc(-c2cc(F)cc(C(F)(F)F)c2)c2[nH]cc(Sc3ccc(C(F)(F)F)cc3)c12 ; O=C(O)CC12CC(NC(=O)c3ccc(-c4cc(F)cc(C(F)(F)F)c4)c4[nH]cc(Sc5ccc(C(F)(F)F)cc5)c34)(C1)C2 ; O=C(NC1CC2(C1)CC(C(=O)O)C2)c1ccc(-c2cc(F)cc(C(F)(F)F)c2)c2[nH]cc(Sc3ccc(C(F)(F)F)cc3)c12]

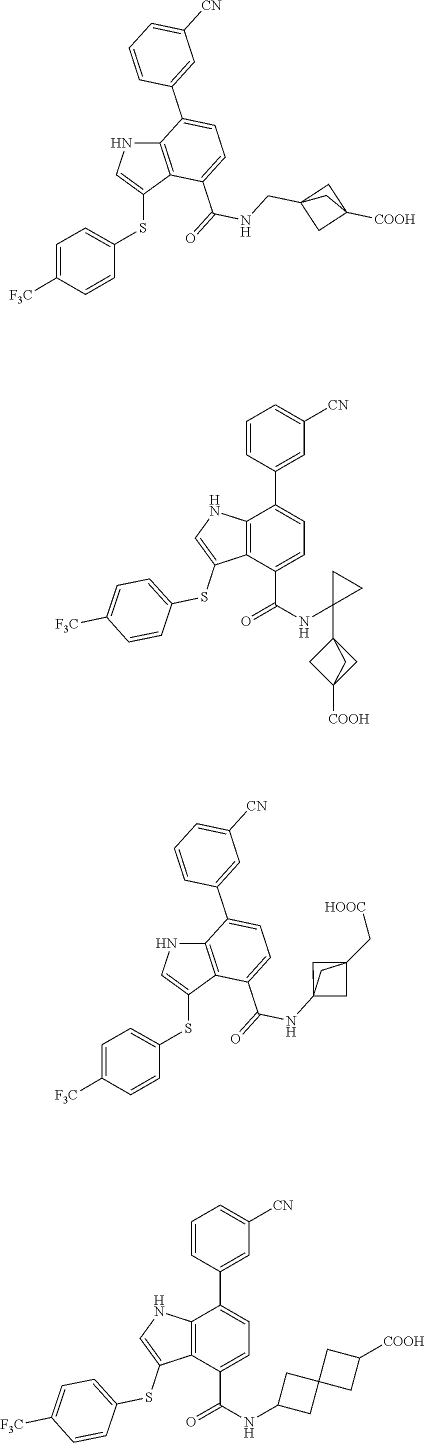 [SMILES: N#Cc1cccc(-c2ccc(C(=O)NCC34CC(C(=O)O)(C3)C4)c3c(Sc4ccc(C(F)(F)F)cc4)c[nH]c23)c1 ; N#Cc1cccc(-c2ccc(C(=O)NC3(C45CC(C(=O)O)(C4)C5)CC3)c3c(Sc4ccc(C(F)(F)F)cc4)c[nH]c23)c1 ; N#Cc1cccc(-c2ccc(C(=O)NC34CC(CC(=O)O)(C3)C4)c3c(Sc4ccc(C(F)(F)F)cc4)c[nH]c23)c1 ; N#Cc1cccc(-c2ccc(C(=O)NC3CC4(C3)CC(C(=O)O)C4)c3c(Sc4ccc(C(F)(F)F)cc4)c[nH]c23)c1]

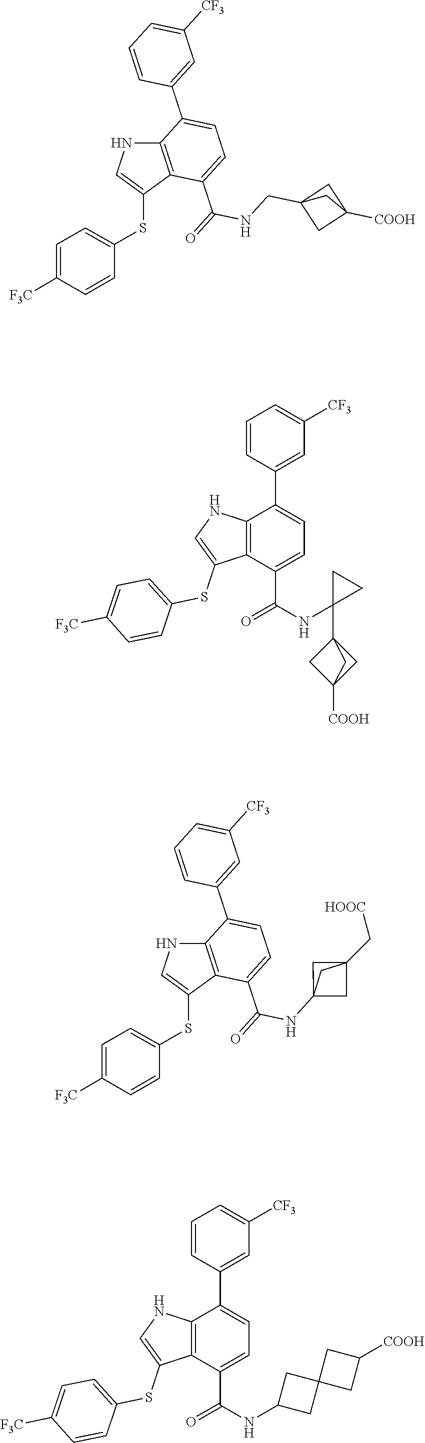 [SMILES: O=C(NCC12CC(C(=O)O)(C1)C2)c1ccc(-c2cccc(C(F)(F)F)c2)c2[nH]cc(Sc3ccc(C(F)(F)F)cc3)c12 ; O=C(NC1(C23CC(C(=O)O)(C2)C3)CC1)c1ccc(-c2cccc(C(F)(F)F)c2)c2[nH]cc(Sc3ccc(C(F)(F)F)cc3)c12 ; O=C(O)CC12CC(NC(=O)c3ccc(-c4cccc(C(F)(F)F)c4)c4[nH]cc(Sc5ccc(C(F)(F)F)cc5)c34)(C1)C2 ; O=C(NC1CC2(C1)CC(C(=O)O)C2)c1ccc(-c2cccc(C(F)(F)F)c2)c2[nH]cc(Sc3ccc(C(F)(F)F)cc3)c12]

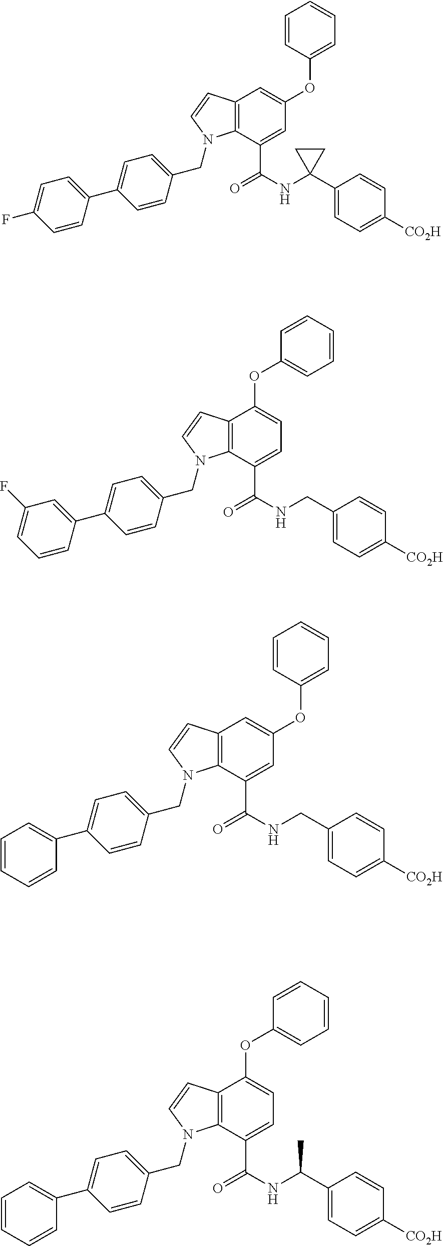 [SMILES: O=C(O)c1ccc(C2(NC(=O)c3cc(Oc4ccccc4)cc4ccn(Cc5ccc(-c6ccc(F)cc6)cc5)c34)CC2)cc1 ; O=C(O)c1ccc(CNC(=O)c2ccc(Oc3ccccc3)c3ccn(Cc4ccc(-c5cccc(F)c5)cc4)c23)cc1 ; O=C(O)c1ccc(CNC(=O)c2cc(Oc3ccccc3)cc3ccn(Cc4ccc(-c5ccccc5)cc4)c23)cc1 ; C[C@H](NC(=O)c1ccc(Oc2ccccc2)c2ccn(Cc3ccc(-c4ccccc4)cc3)c12)c1ccc(C(=O)O)cc1]

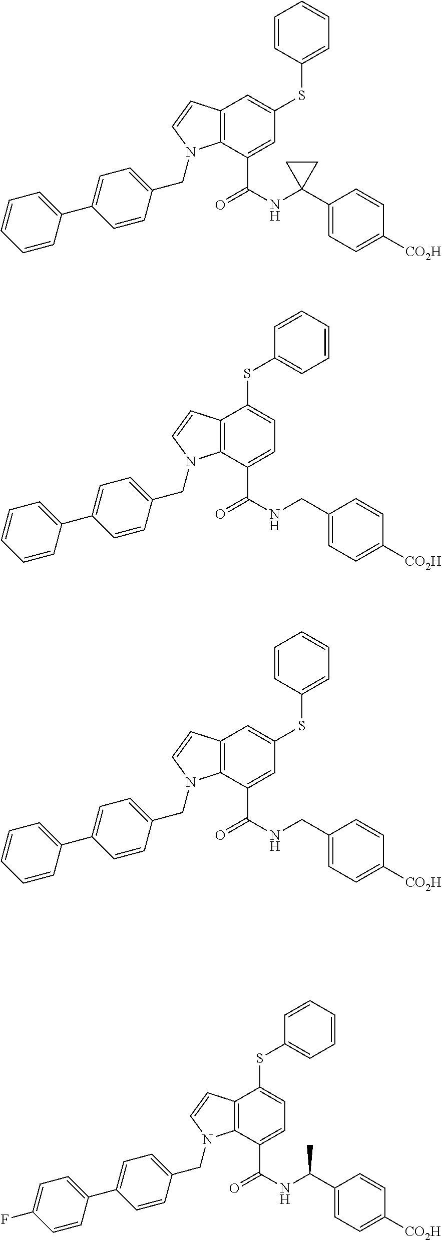 [SMILES: O=C(O)c1ccc(C2(NC(=O)c3cc(Sc4ccccc4)cc4ccn(Cc5ccc(-c6ccccc6)cc5)c34)CC2)cc1 ; O=C(O)c1ccc(CNC(=O)c2ccc(Sc3ccccc3)c3ccn(Cc4ccc(-c5ccccc5)cc4)c23)cc1 ; O=C(O)c1ccc(CNC(=O)c2cc(Sc3ccccc3)cc3ccn(Cc4ccc(-c5ccccc5)cc4)c23)cc1 ; C[C@H](NC(=O)c1ccc(Sc2ccccc2)c2ccn(Cc3ccc(-c4ccc(F)cc4)cc3)c12)c1ccc(C(=O)O)cc1]

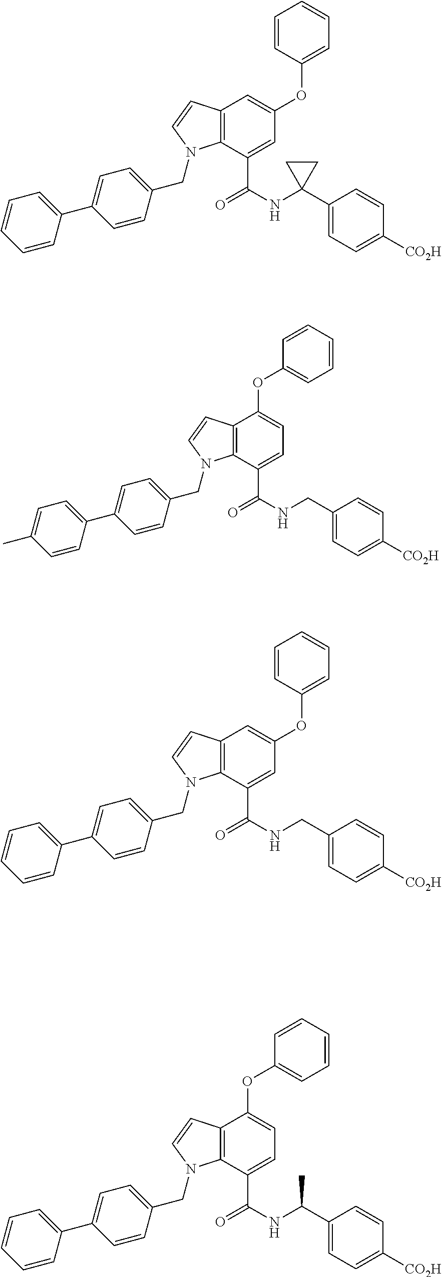 [SMILES: O=C(O)c1ccc(C2(NC(=O)c3cc(Oc4ccccc4)cc4ccn(Cc5ccc(-c6ccccc6)cc5)c34)CC2)cc1 ; Cc1ccc(-c2ccc(Cn3ccc4c(Oc5ccccc5)ccc(C(=O)NCc5ccc(C(=O)O)cc5)c43)cc2)cc1 ; O=C(O)c1ccc(CNC(=O)c2cc(Oc3ccccc3)cc3ccn(Cc4ccc(-c5ccccc5)cc4)c23)cc1 ; C[C@H](NC(=O)c1ccc(Oc2ccccc2)c2ccn(Cc3ccc(-c4ccccc4)cc3)c12)c1ccc(C(=O)O)cc1]

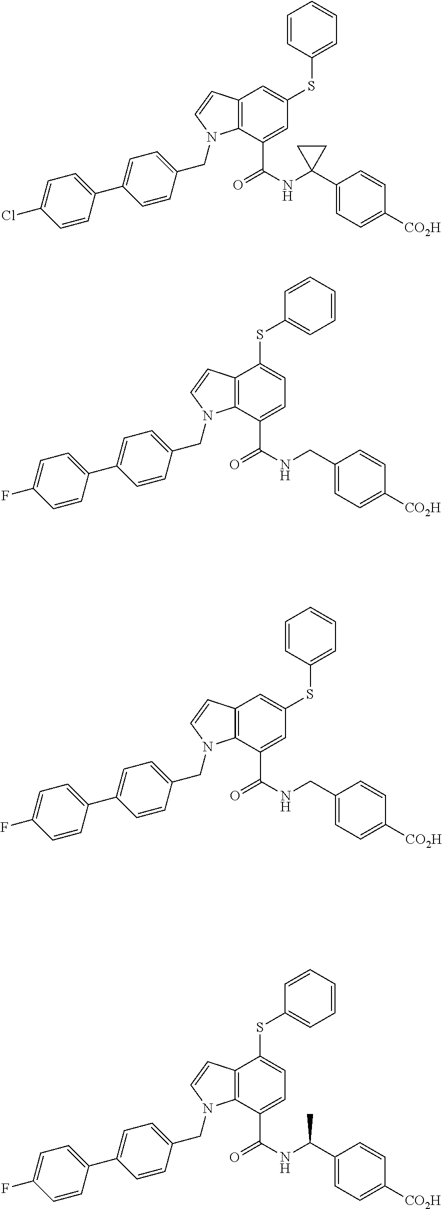 [SMILES: O=C(O)c1ccc(C2(NC(=O)c3cc(Sc4ccccc4)cc4ccn(Cc5ccc(-c6ccc(Cl)cc6)cc5)c34)CC2)cc1 ; O=C(O)c1ccc(CNC(=O)c2ccc(Sc3ccccc3)c3ccn(Cc4ccc(-c5ccc(F)cc5)cc4)c23)cc1 ; O=C(O)c1ccc(CNC(=O)c2cc(Sc3ccccc3)cc3ccn(Cc4ccc(-c5ccc(F)cc5)cc4)c23)cc1 ; C[C@H](NC(=O)c1ccc(Sc2ccccc2)c2ccn(Cc3ccc(-c4ccc(F)cc4)cc3)c12)c1ccc(C(=O)O)cc1]

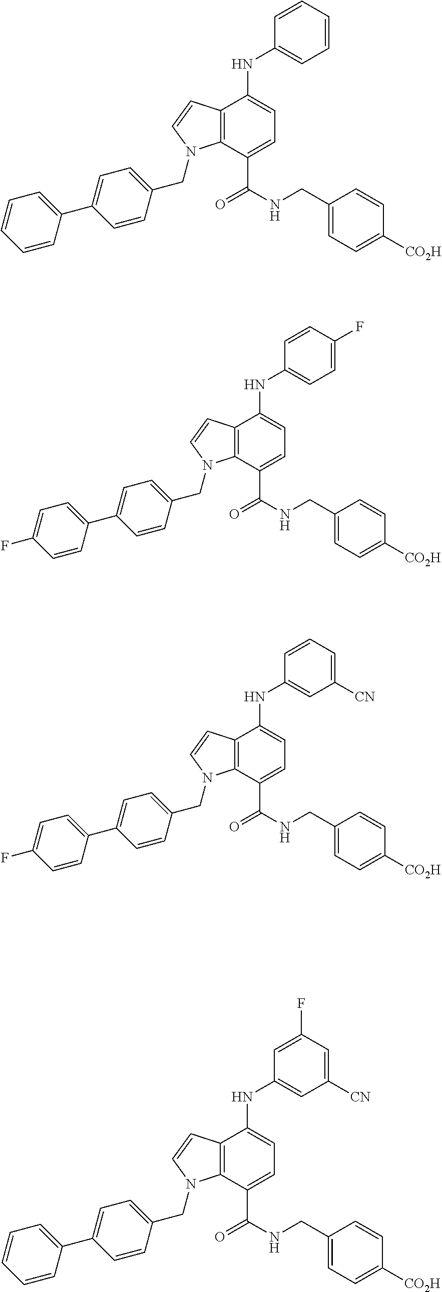 [SMILES: O=C(O)c1ccc(CNC(=O)c2ccc(Nc3ccccc3)c3ccn(Cc4ccc(-c5ccccc5)cc4)c23)cc1 ; O=C(O)c1ccc(CNC(=O)c2ccc(Nc3ccc(F)cc3)c3ccn(Cc4ccc(-c5ccc(F)cc5)cc4)c23)cc1 ; N#Cc1cccc(Nc2ccc(C(=O)NCc3ccc(C(=O)O)cc3)c3c2ccn3Cc2ccc(-c3ccc(F)cc3)cc2)c1 ; N#Cc1cc(F)cc(Nc2ccc(C(=O)NCc3ccc(C(=O)O)cc3)c3c2ccn3Cc2ccc(-c3ccccc3)cc2)c1]

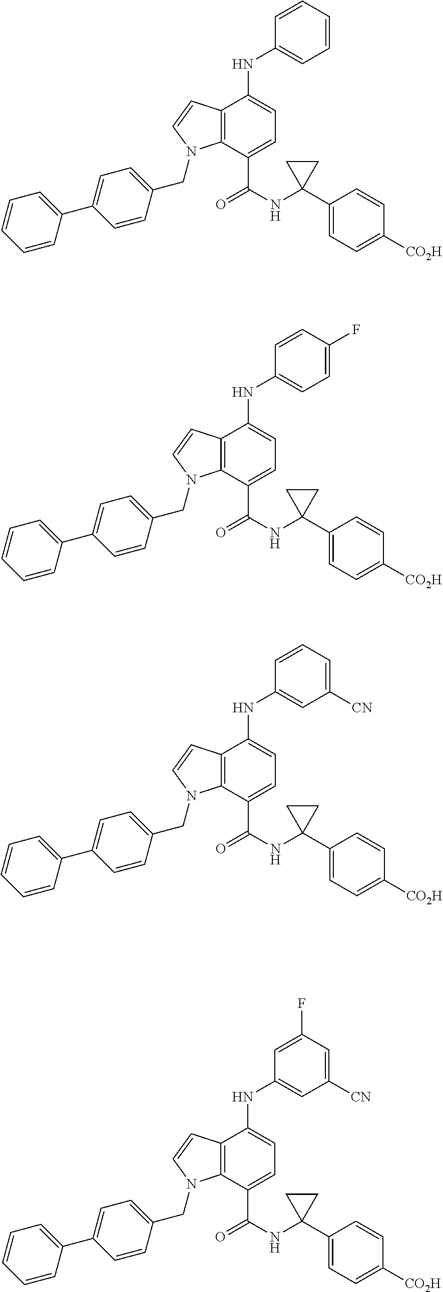 [SMILES: O=C(O)c1ccc(C2(NC(=O)c3ccc(Nc4ccccc4)c4ccn(Cc5ccc(-c6ccccc6)cc5)c34)CC2)cc1 ; O=C(O)c1ccc(C2(NC(=O)c3ccc(Nc4ccc(F)cc4)c4ccn(Cc5ccc(-c6ccccc6)cc5)c34)CC2)cc1 ; N#Cc1cccc(Nc2ccc(C(=O)NC3(c4ccc(C(=O)O)cc4)CC3)c3c2ccn3Cc2ccc(-c3ccccc3)cc2)c1 ; N#Cc1cc(F)cc(Nc2ccc(C(=O)NC3(c4ccc(C(=O)O)cc4)CC3)c3c2ccn3Cc2ccc(-c3ccccc3)cc2)c1]

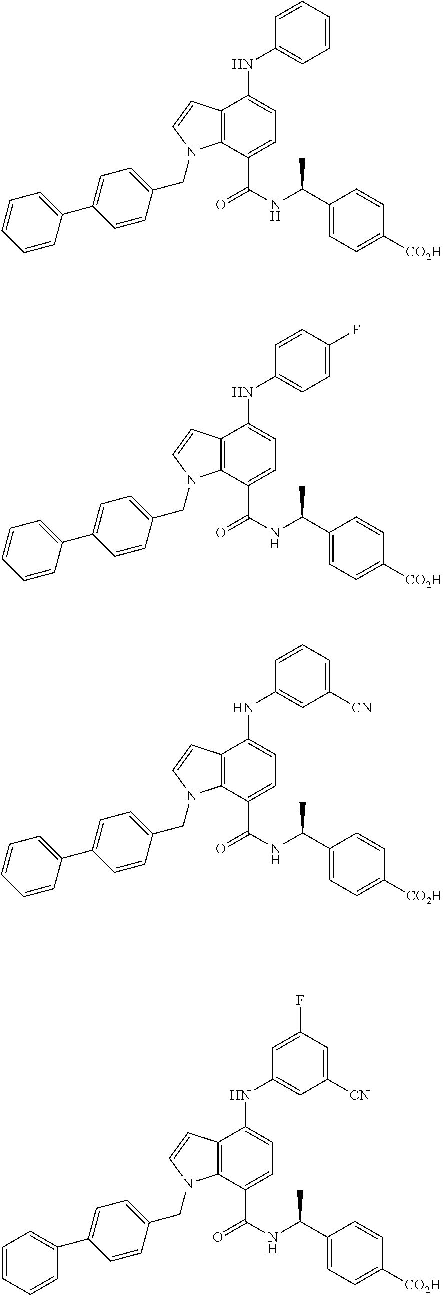 [SMILES: C[C@H](NC(=O)c1ccc(Nc2ccccc2)c2ccn(Cc3ccc(-c4ccccc4)cc3)c12)c1ccc(C(=O)O)cc1 ; C[C@H](NC(=O)c1ccc(Nc2ccc(F)cc2)c2ccn(Cc3ccc(-c4ccccc4)cc3)c12)c1ccc(C(=O)O)cc1 ; C[C@H](NC(=O)c1ccc(Nc2cccc(C#N)c2)c2ccn(Cc3ccc(-c4ccccc4)cc3)c12)c1ccc(C(=O)O)cc1 ; C[C@H](NC(=O)c1ccc(Nc2cc(F)cc(C#N)c2)c2ccn(Cc3ccc(-c4ccccc4)cc3)c12)c1ccc(C(=O)O)cc1]

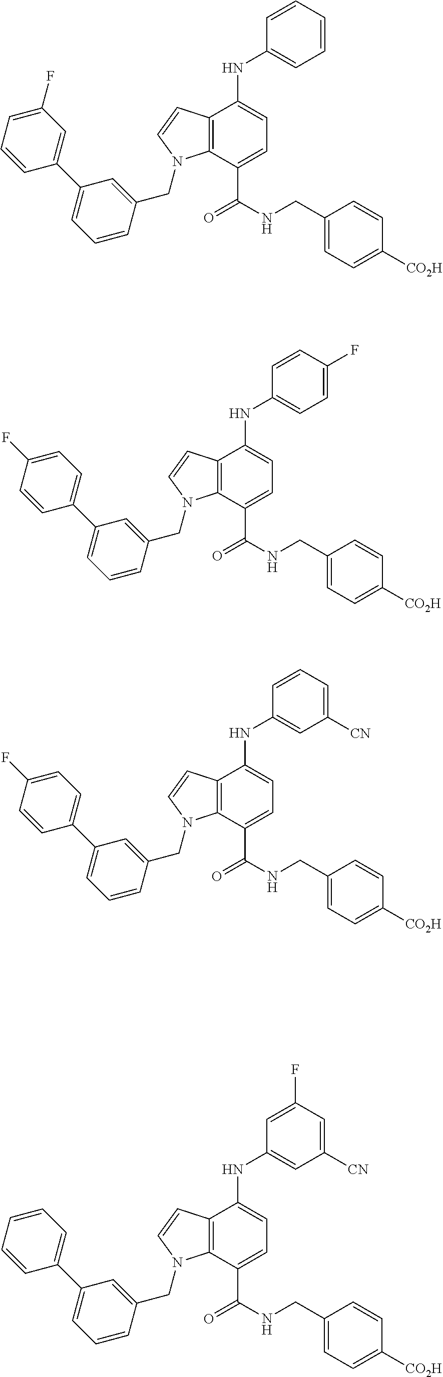 [SMILES: O=C(O)c1ccc(CNC(=O)c2ccc(Nc3ccccc3)c3ccn(Cc4cccc(-c5cccc(F)c5)c4)c23)cc1 ; O=C(O)c1ccc(CNC(=O)c2ccc(Nc3ccc(F)cc3)c3ccn(Cc4cccc(-c5ccc(F)cc5)c4)c23)cc1 ; N#Cc1cccc(Nc2ccc(C(=O)NCc3ccc(C(=O)O)cc3)c3c2ccn3Cc2cccc(-c3ccc(F)cc3)c2)c1 ; N#Cc1cc(F)cc(Nc2ccc(C(=O)NCc3ccc(C(=O)O)cc3)c3c2ccn3Cc2cccc(-c3ccccc3)c2)c1]

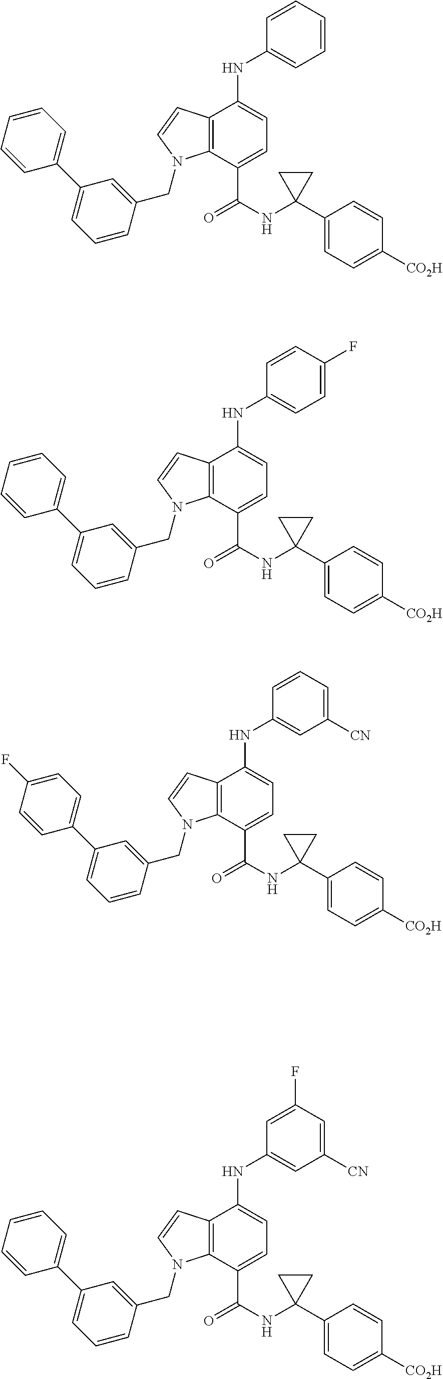 [SMILES: O=C(O)c1ccc(C2(NC(=O)c3ccc(Nc4ccccc4)c4ccn(Cc5cccc(-c6ccccc6)c5)c34)CC2)cc1 ; O=C(O)c1ccc(C2(NC(=O)c3ccc(Nc4ccc(F)cc4)c4ccn(Cc5cccc(-c6ccccc6)c5)c34)CC2)cc1 ; N#Cc1cccc(Nc2ccc(C(=O)NC3(c4ccc(C(=O)O)cc4)CC3)c3c2ccn3Cc2cccc(-c3ccc(F)cc3)c2)c1 ; N#Cc1cc(F)cc(Nc2ccc(C(=O)NC3(c4ccc(C(=O)O)cc4)CC3)c3c2ccn3Cc2cccc(-c3ccccc3)c2)c1]

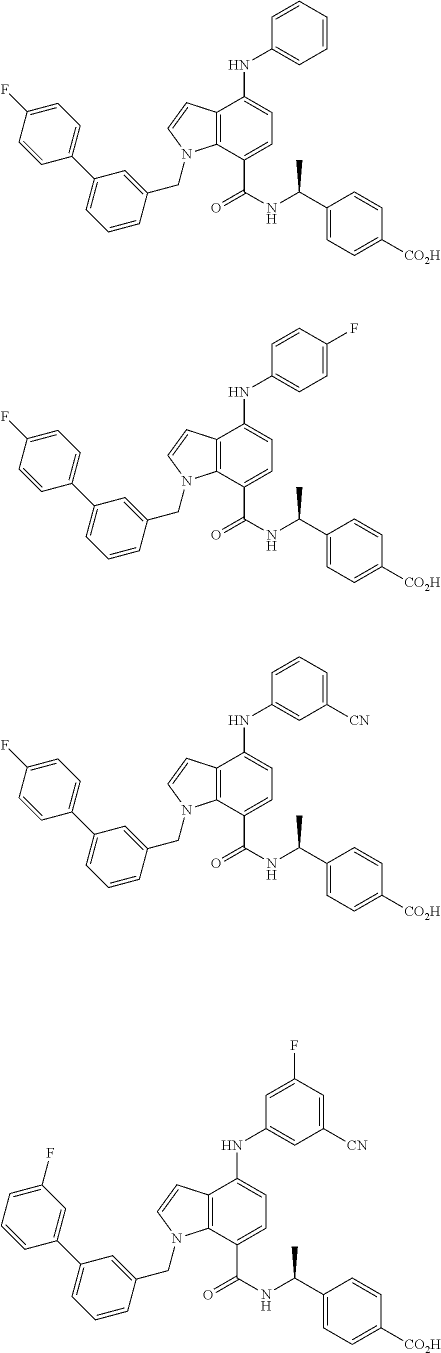 [SMILES: C[C@H](NC(=O)c1ccc(Nc2ccccc2)c2ccn(Cc3cccc(-c4ccc(F)cc4)c3)c12)c1ccc(C(=O)O)cc1 ; C[C@H](NC(=O)c1ccc(Nc2ccc(F)cc2)c2ccn(Cc3cccc(-c4ccc(F)cc4)c3)c12)c1ccc(C(=O)O)cc1 ; C[C@H](NC(=O)c1ccc(Nc2cccc(C#N)c2)c2ccn(Cc3cccc(-c4ccc(F)cc4)c3)c12)c1ccc(C(=O)O)cc1 ; C[C@H](NC(=O)c1ccc(Nc2cc(F)cc(C#N)c2)c2ccn(Cc3cccc(-c4cccc(F)c4)c3)c12)c1ccc(C(=O)O)cc1]

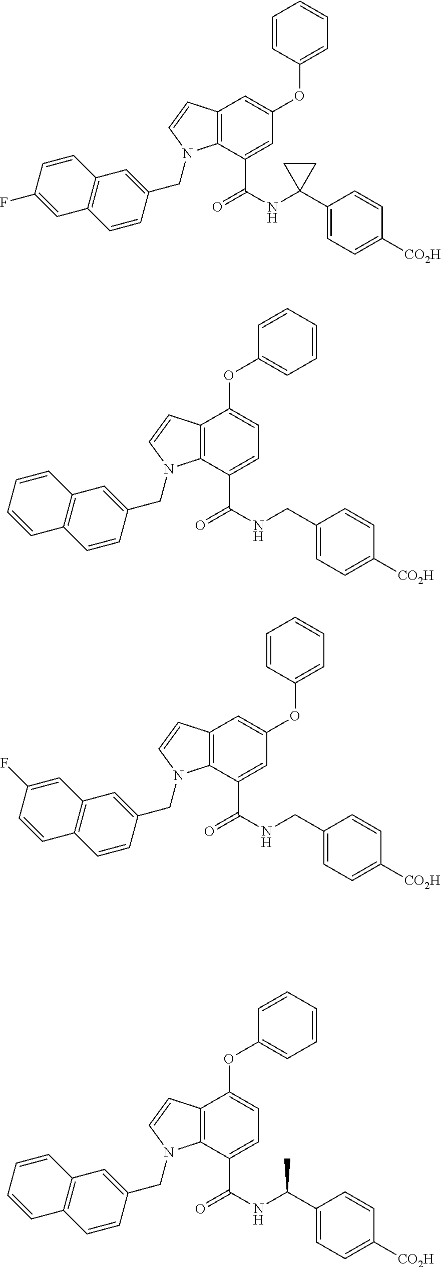 [SMILES: O=C(O)c1ccc(C2(NC(=O)c3cc(Oc4ccccc4)cc4ccn(Cc5ccc6cc(F)ccc6c5)c34)CC2)cc1 ; O=C(O)c1ccc(CNC(=O)c2ccc(Oc3ccccc3)c3ccn(Cc4ccc5ccccc5c4)c23)cc1 ; O=C(O)c1ccc(CNC(=O)c2cc(Oc3ccccc3)cc3ccn(Cc4ccc5ccc(F)cc5c4)c23)cc1 ; C[C@H](NC(=O)c1ccc(Oc2ccccc2)c2ccn(Cc3ccc4ccccc4c3)c12)c1ccc(C(=O)O)cc1]

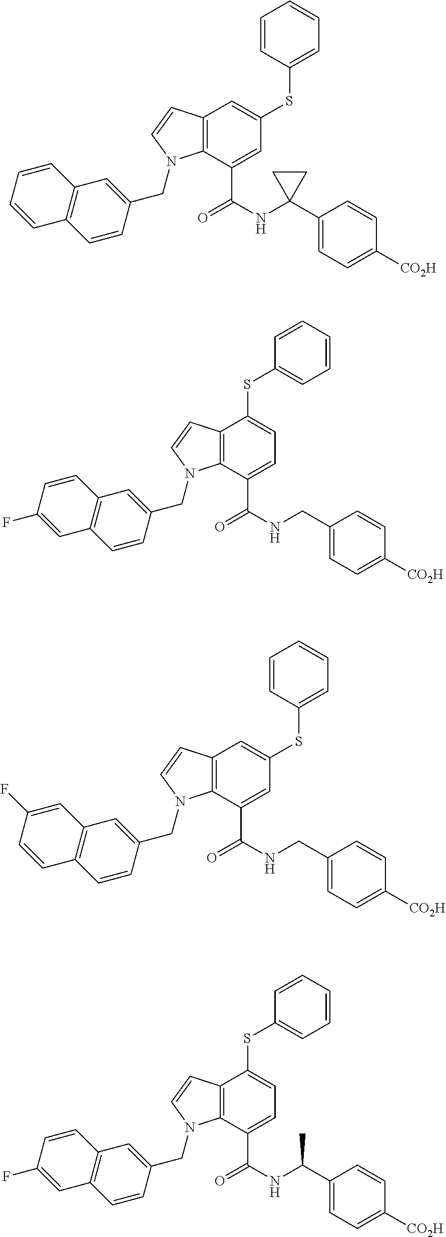 [SMILES: O=C(O)c1ccc(C2(NC(=O)c3cc(Sc4ccccc4)cc4ccn(Cc5ccc6ccccc6c5)c34)CC2)cc1 ; O=C(O)c1ccc(CNC(=O)c2ccc(Sc3ccccc3)c3ccn(Cc4ccc5cc(F)ccc5c4)c23)cc1 ; O=C(O)c1ccc(CNC(=O)c2cc(Sc3ccccc3)cc3ccn(Cc4ccc5ccc(F)cc5c4)c23)cc1 ; C[C@H](NC(=O)c1ccc(Sc2ccccc2)c2ccn(Cc3ccc4cc(F)ccc4c3)c12)c1ccc(C(=O)O)cc1]

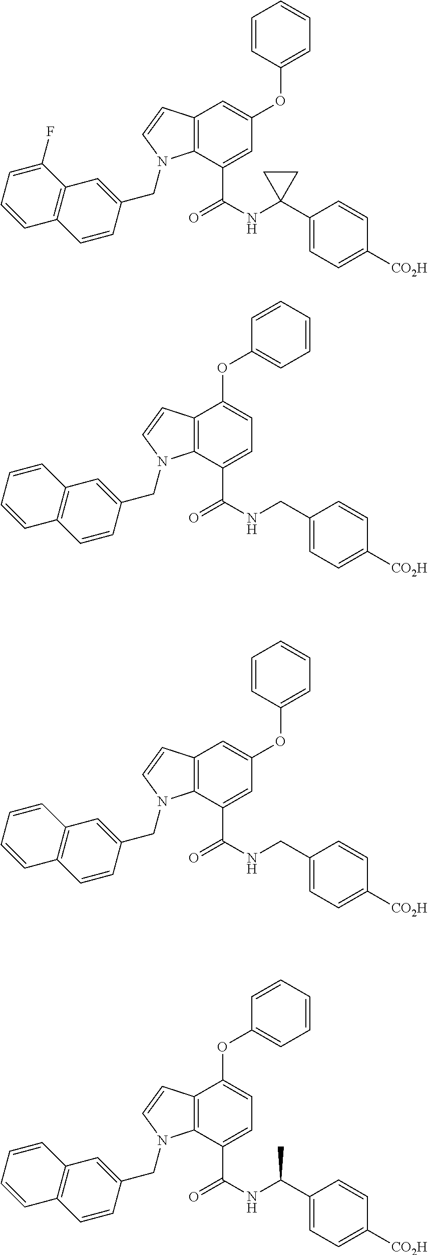 [SMILES: O=C(O)c1ccc(C2(NC(=O)c3cc(Oc4ccccc4)cc4ccn(Cc5ccc6cccc(F)c6c5)c34)CC2)cc1 ; O=C(O)c1ccc(CNC(=O)c2ccc(Oc3ccccc3)c3ccn(Cc4ccc5ccccc5c4)c23)cc1 ; O=C(O)c1ccc(CNC(=O)c2cc(Oc3ccccc3)cc3ccn(Cc4ccc5ccccc5c4)c23)cc1 ; C[C@H](NC(=O)c1ccc(Oc2ccccc2)c2ccn(Cc3ccc4ccccc4c3)c12)c1ccc(C(=O)O)cc1]

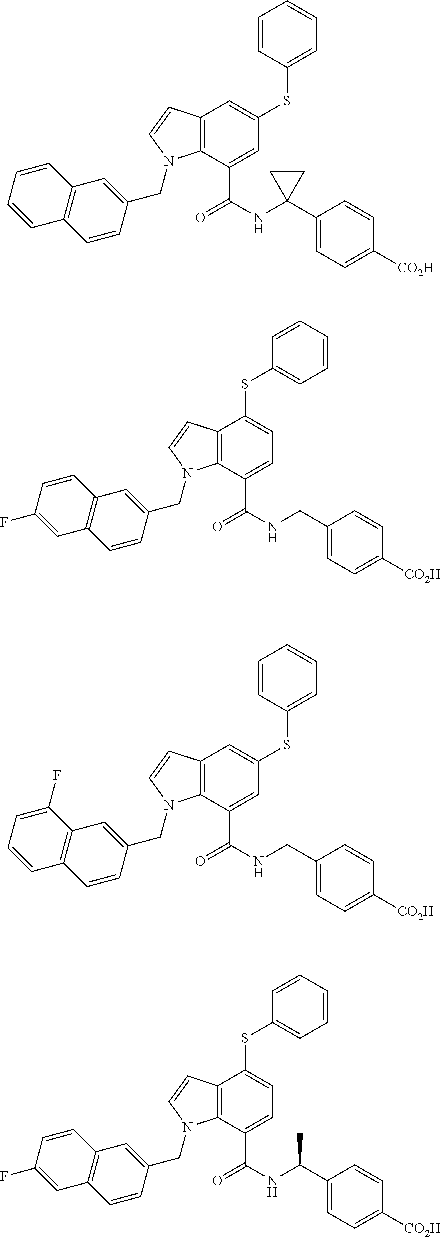 [SMILES: O=C(O)c1ccc(C2(NC(=O)c3cc(Sc4ccccc4)cc4ccn(Cc5ccc6ccccc6c5)c34)CC2)cc1 ; O=C(O)c1ccc(CNC(=O)c2ccc(Sc3ccccc3)c3ccn(Cc4ccc5cc(F)ccc5c4)c23)cc1 ; O=C(O)c1ccc(CNC(=O)c2cc(Sc3ccccc3)cc3ccn(Cc4ccc5cccc(F)c5c4)c23)cc1 ; C[C@H](NC(=O)c1ccc(Sc2ccccc2)c2ccn(Cc3ccc4cc(F)ccc4c3)c12)c1ccc(C(=O)O)cc1]

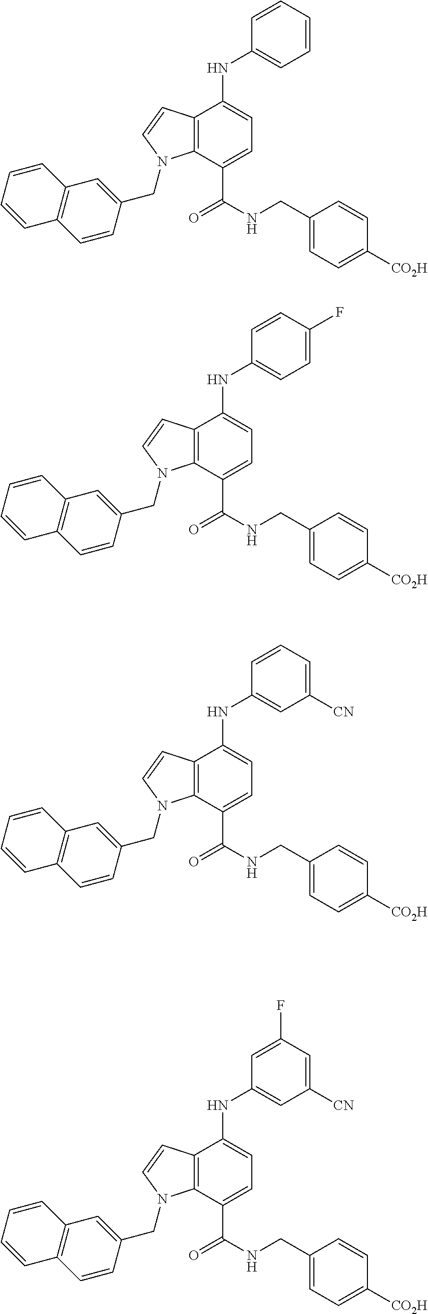

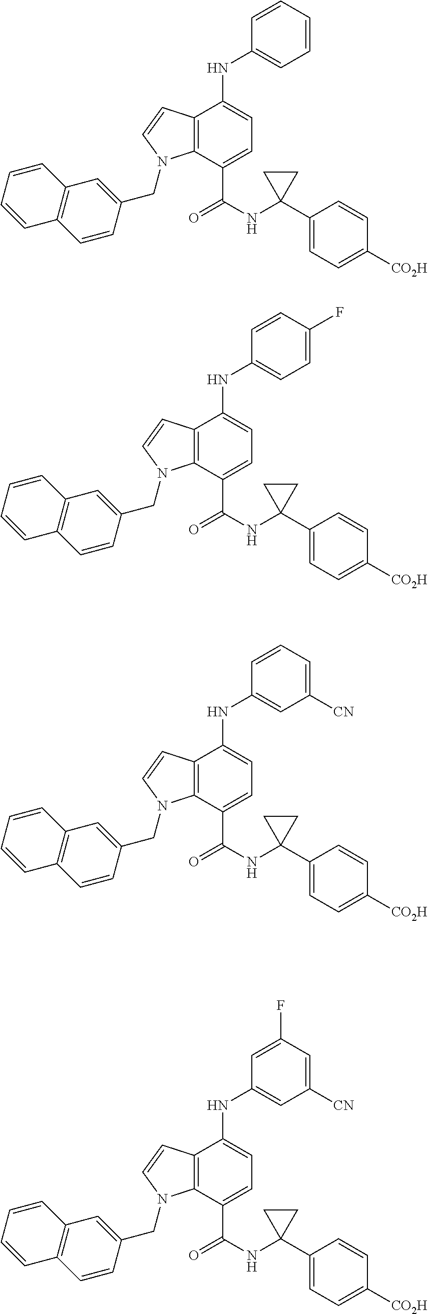

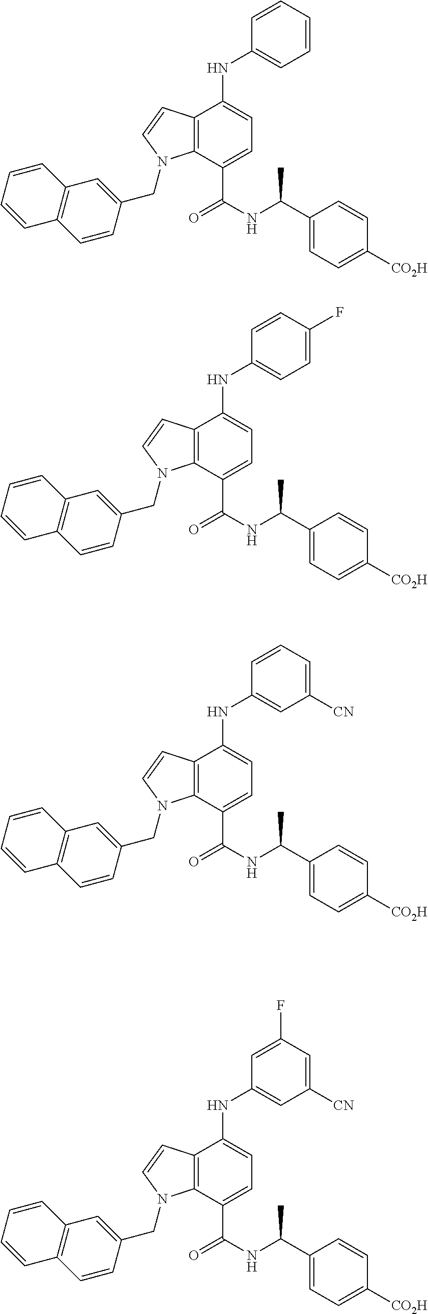

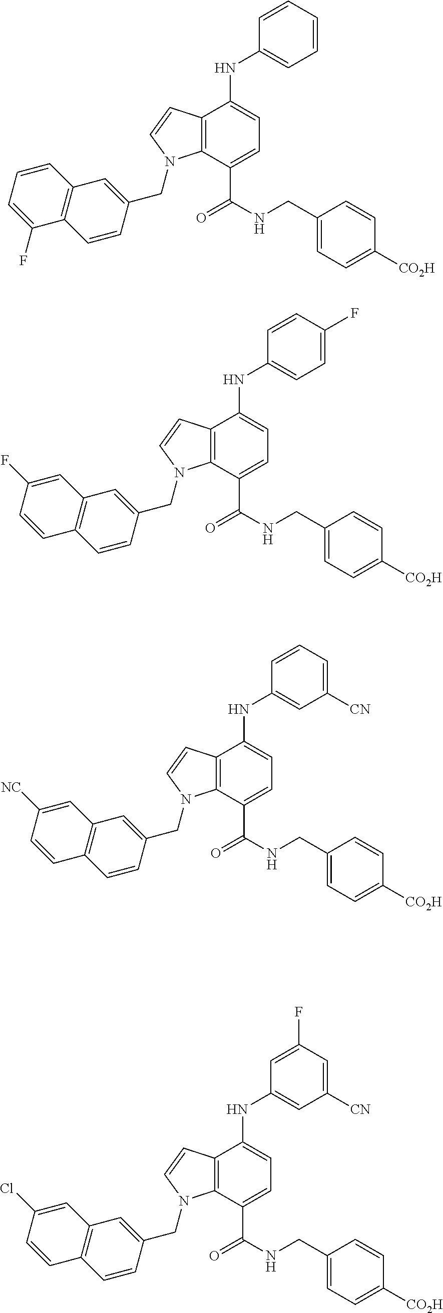 [SMILES: O=C(O)c1ccc(CNC(=O)c2ccc(Nc3ccccc3)c3ccn(Cc4ccc5c(F)cccc5c4)c23)cc1 ; O=C(O)c1ccc(CNC(=O)c2ccc(Nc3ccc(F)cc3)c3ccn(Cc4ccc5ccc(F)cc5c4)c23)cc1 ; N#Cc1cccc(Nc2ccc(C(=O)NCc3ccc(C(=O)O)cc3)c3c2ccn3Cc2ccc3ccc(C#N)cc3c2)c1 ; N#Cc1cc(F)cc(Nc2ccc(C(=O)NCc3ccc(C(=O)O)cc3)c3c2ccn3Cc2ccc3ccc(Cl)cc3c2)c1]

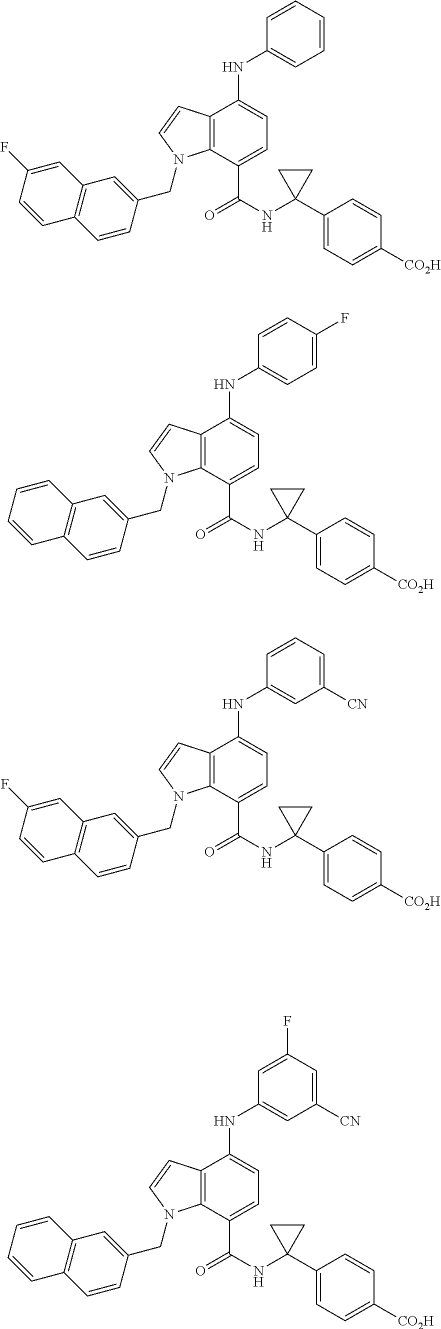 [SMILES: O=C(O)c1ccc(C2(NC(=O)c3ccc(Nc4ccccc4)c4ccn(Cc5ccc6ccc(F)cc6c5)c34)CC2)cc1 ; O=C(O)c1ccc(C2(NC(=O)c3ccc(Nc4ccc(F)cc4)c4ccn(Cc5ccc6ccccc6c5)c34)CC2)cc1 ; N#Cc1cccc(Nc2ccc(C(=O)NC3(c4ccc(C(=O)O)cc4)CC3)c3c2ccn3Cc2ccc3ccc(F)cc3c2)c1 ; N#Cc1cc(F)cc(Nc2ccc(C(=O)NC3(c4ccc(C(=O)O)cc4)CC3)c3c2ccn3Cc2ccc3ccccc3c2)c1]

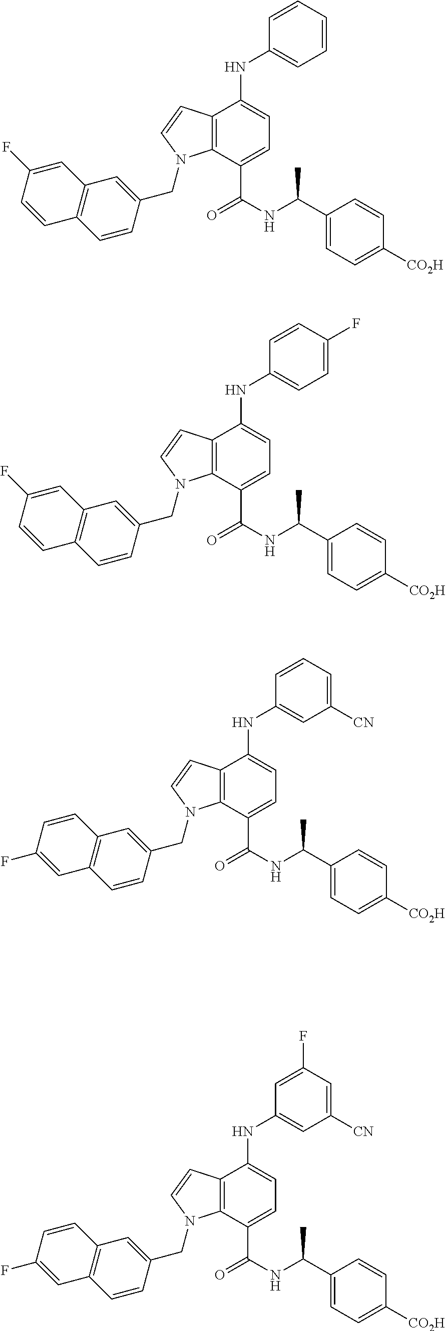 [SMILES: C[C@H](NC(=O)c1ccc(Nc2ccccc2)c2ccn(Cc3ccc4ccc(F)cc4c3)c12)c1ccc(C(=O)O)cc1 ; C[C@H](NC(=O)c1ccc(Nc2ccc(F)cc2)c2ccn(Cc3ccc4ccc(F)cc4c3)c12)c1ccc(C(=O)O)cc1 ; C[C@H](NC(=O)c1ccc(Nc2cccc(C#N)c2)c2ccn(Cc3ccc4cc(F)ccc4c3)c12)c1ccc(C(=O)O)cc1 ; C[C@H](NC(=O)c1ccc(Nc2cc(F)cc(C#N)c2)c2ccn(Cc3ccc4cc(F)ccc4c3)c12)c1ccc(C(=O)O)cc1]

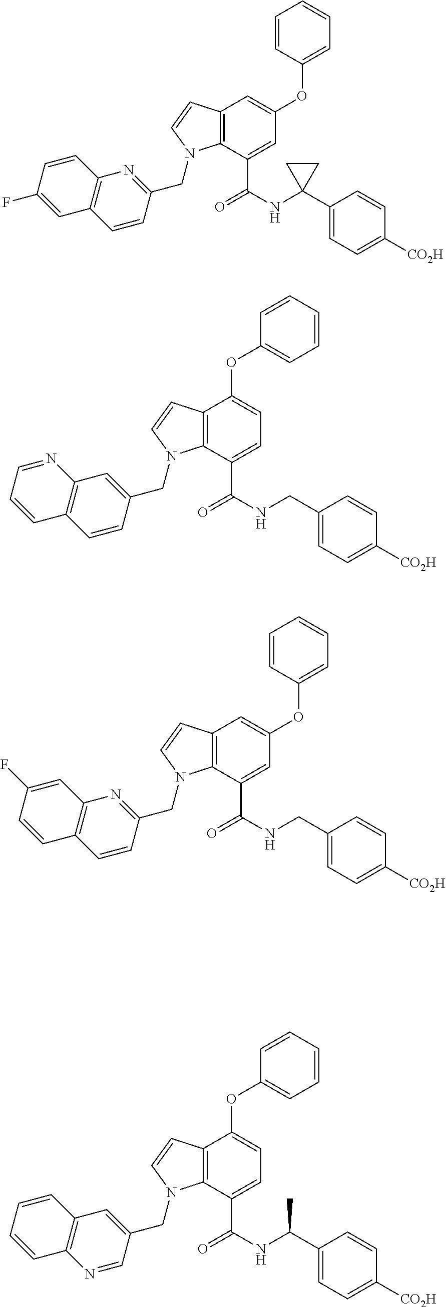 [SMILES: O=C(O)c1ccc(C2(NC(=O)c3cc(Oc4ccccc4)cc4ccn(Cc5ccc6cc(F)ccc6n5)c34)CC2)cc1 ; O=C(O)c1ccc(CNC(=O)c2ccc(Oc3ccccc3)c3ccn(Cc4ccc5cccnc5c4)c23)cc1 ; O=C(O)c1ccc(CNC(=O)c2cc(Oc3ccccc3)cc3ccn(Cc4ccc5ccc(F)cc5n4)c23)cc1 ; C[C@H](NC(=O)c1ccc(Oc2ccccc2)c2ccn(Cc3cnc4ccccc4c3)c12)c1ccc(C(=O)O)cc1]

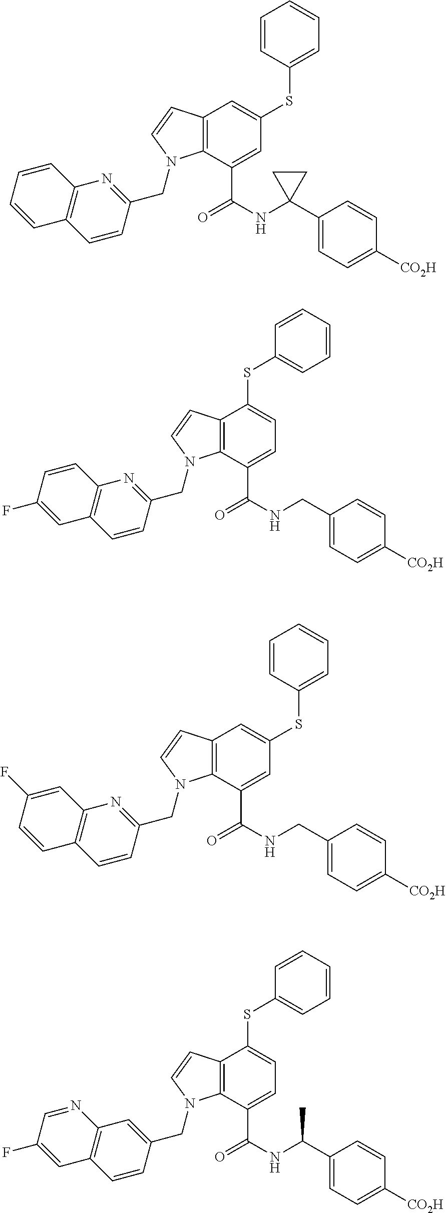 [SMILES: O=C(O)c1ccc(C2(NC(=O)c3cc(Sc4ccccc4)cc4ccn(Cc5ccc6ccccc6n5)c34)CC2)cc1 ; O=C(O)c1ccc(CNC(=O)c2ccc(Sc3ccccc3)c3ccn(Cc4ccc5cc(F)ccc5n4)c23)cc1 ; O=C(O)c1ccc(CNC(=O)c2cc(Sc3ccccc3)cc3ccn(Cc4ccc5ccc(F)cc5n4)c23)cc1 ; C[C@H](NC(=O)c1ccc(Sc2ccccc2)c2ccn(Cc3ccc4cc(F)cnc4c3)c12)c1ccc(C(=O)O)cc1]

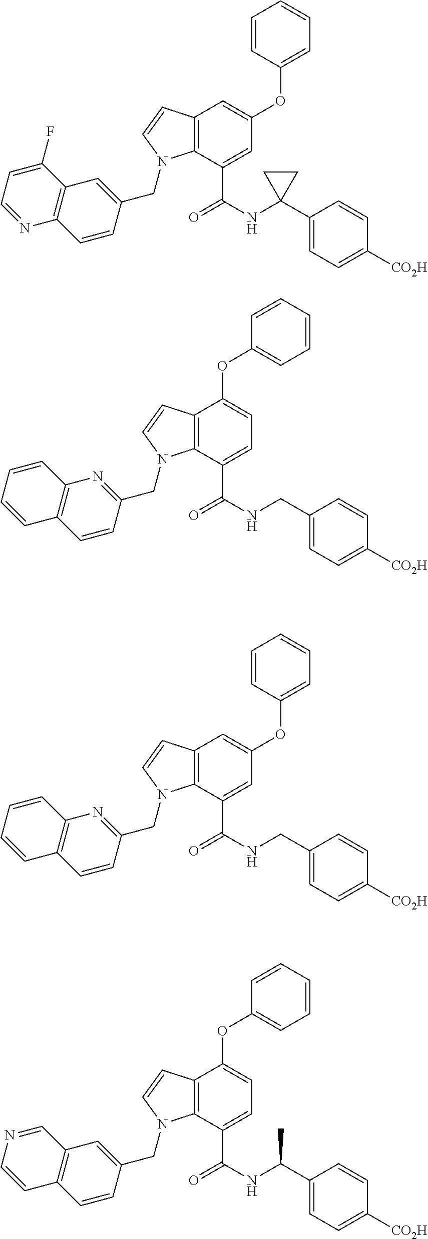 [SMILES: O=C(O)c1ccc(C2(NC(=O)c3cc(Oc4ccccc4)cc4ccn(Cc5ccc6nccc(F)c6c5)c34)CC2)cc1 ; O=C(O)c1ccc(CNC(=O)c2ccc(Oc3ccccc3)c3ccn(Cc4ccc5ccccc5n4)c23)cc1 ; O=C(O)c1ccc(CNC(=O)c2cc(Oc3ccccc3)cc3ccn(Cc4ccc5ccccc5n4)c23)cc1 ; C[C@H](NC(=O)c1ccc(Oc2ccccc2)c2ccn(Cc3ccc4ccncc4c3)c12)c1ccc(C(=O)O)cc1]

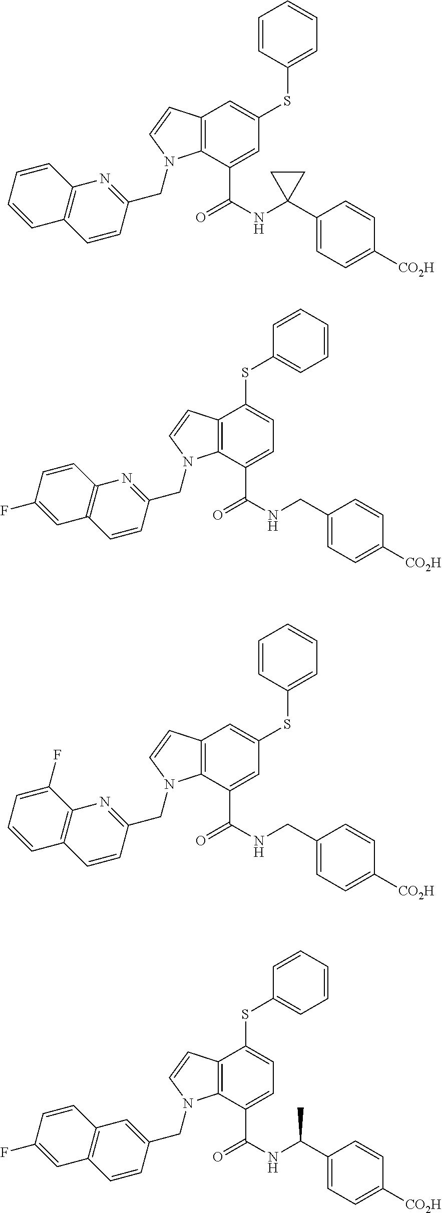 [SMILES: O=C(O)c1ccc(C2(NC(=O)c3cc(Sc4ccccc4)cc4ccn(Cc5ccc6ccccc6n5)c34)CC2)cc1 ; O=C(O)c1ccc(CNC(=O)c2ccc(Sc3ccccc3)c3ccn(Cc4ccc5cc(F)ccc5n4)c23)cc1 ; O=C(O)c1ccc(CNC(=O)c2cc(Sc3ccccc3)cc3ccn(Cc4ccc5cccc(F)c5n4)c23)cc1 ; C[C@H](NC(=O)c1ccc(Sc2ccccc2)c2ccn(Cc3ccc4cc(F)ccc4c3)c12)c1ccc(C(=O)O)cc1]

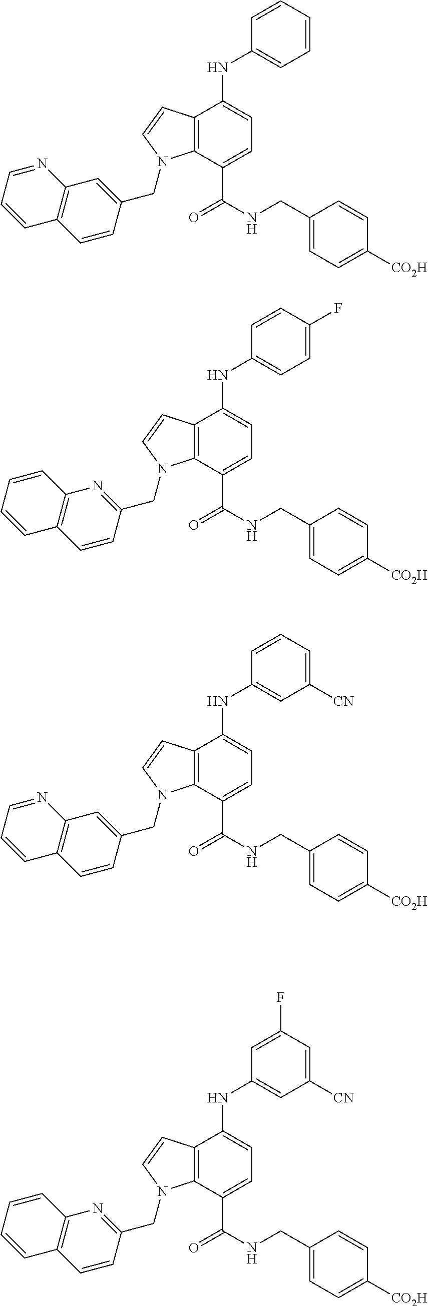

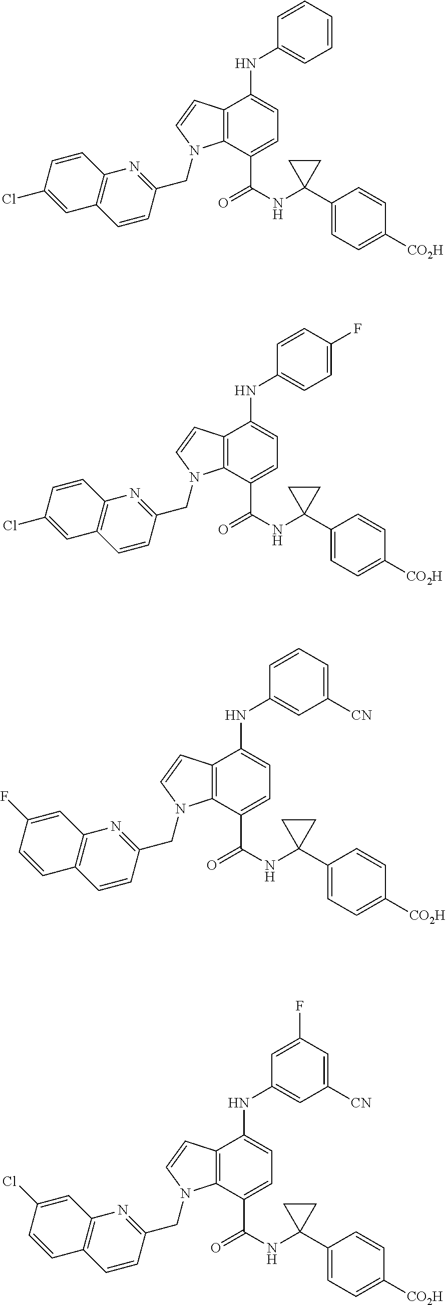 [SMILES: O=C(O)c1ccc(C2(NC(=O)c3ccc(Nc4ccccc4)c4ccn(Cc5ccc6cc(Cl)ccc6n5)c34)CC2)cc1 ; O=C(O)c1ccc(C2(NC(=O)c3ccc(Nc4ccc(F)cc4)c4ccn(Cc5ccc6cc(Cl)ccc6n5)c34)CC2)cc1 ; N#Cc1cccc(Nc2ccc(C(=O)NC3(c4ccc(C(=O)O)cc4)CC3)c3c2ccn3Cc2ccc3ccc(F)cc3n2)c1 ; N#Cc1cc(F)cc(Nc2ccc(C(=O)NC3(c4ccc(C(=O)O)cc4)CC3)c3c2ccn3Cc2ccc3ccc(Cl)cc3n2)c1]

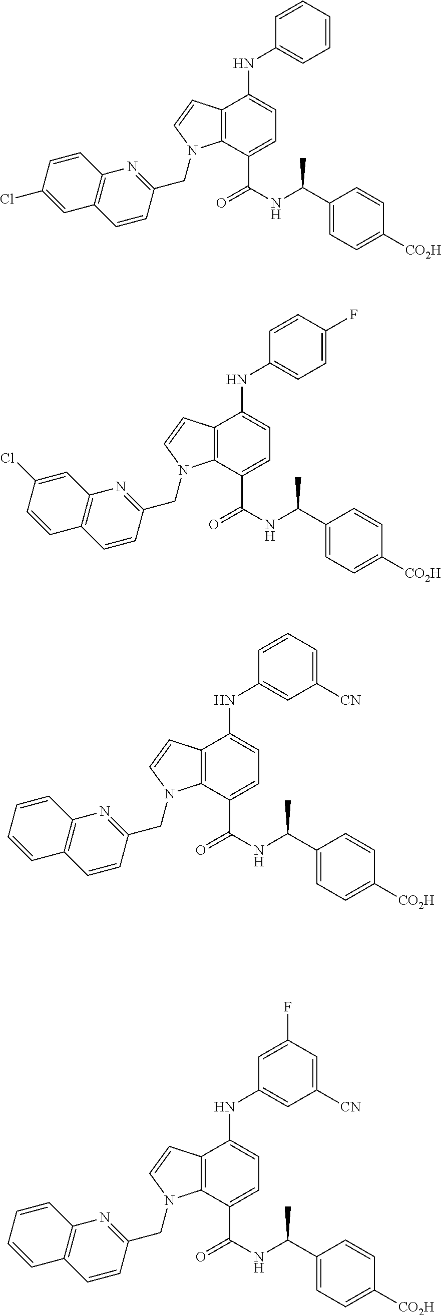 [SMILES: C[C@H](NC(=O)c1ccc(Nc2ccccc2)c2ccn(Cc3ccc4cc(Cl)ccc4n3)c12)c1ccc(C(=O)O)cc1 ; C[C@H](NC(=O)c1ccc(Nc2ccc(F)cc2)c2ccn(Cc3ccc4ccc(Cl)cc4n3)c12)c1ccc(C(=O)O)cc1 ; C[C@H](NC(=O)c1ccc(Nc2cccc(C#N)c2)c2ccn(Cc3ccc4ccccc4n3)c12)c1ccc(C(=O)O)cc1 ; C[C@H](NC(=O)c1ccc(Nc2cc(F)cc(C#N)c2)c2ccn(Cc3ccc4ccccc4n3)c12)c1ccc(C(=O)O)cc1]

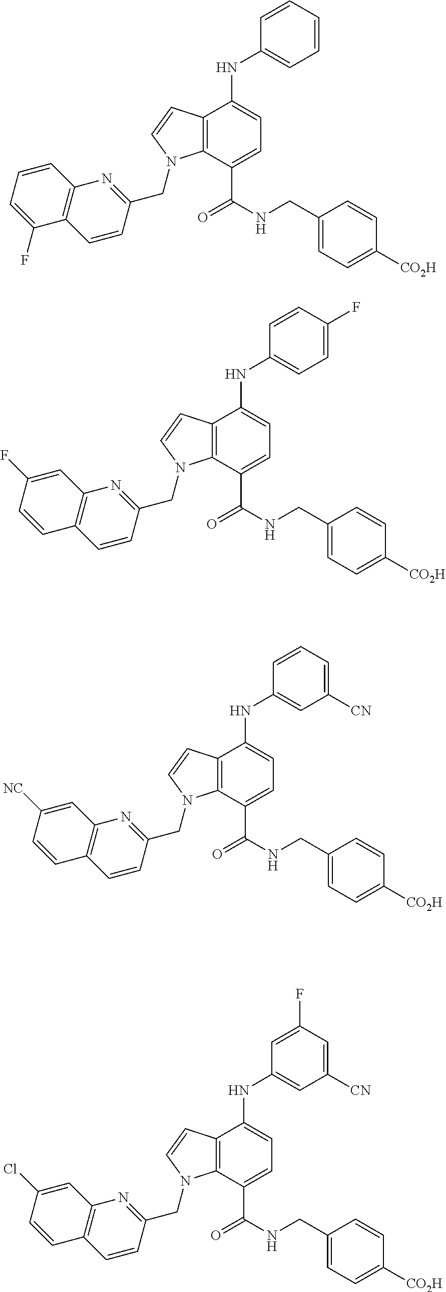

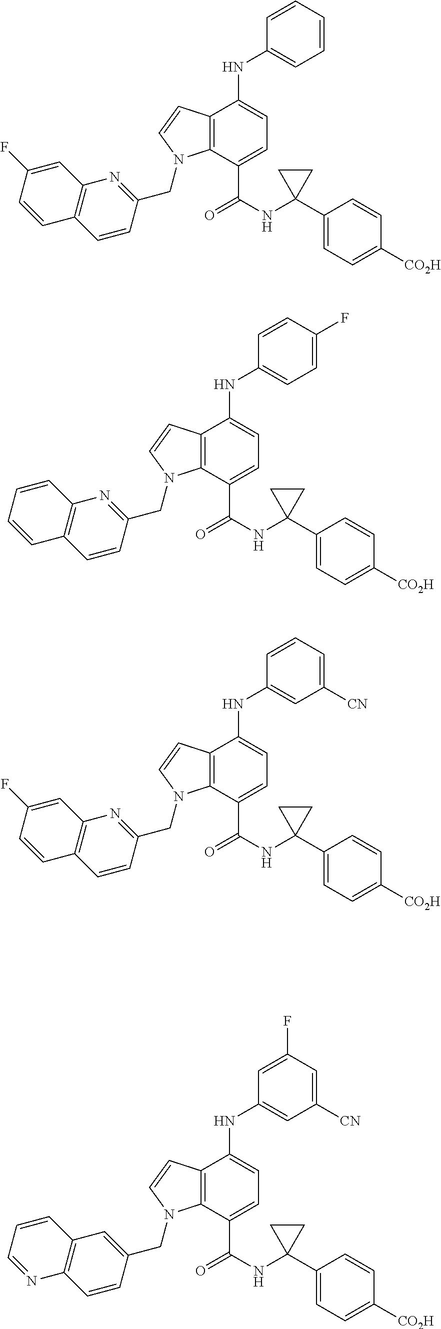 [SMILES: O=C(O)c1ccc(C2(NC(=O)c3ccc(Nc4ccccc4)c4ccn(Cc5ccc6ccc(F)cc6n5)c34)CC2)cc1 ; O=C(O)c1ccc(C2(NC(=O)c3ccc(Nc4ccc(F)cc4)c4ccn(Cc5ccc6ccccc6n5)c34)CC2)cc1 ; N#Cc1cccc(Nc2ccc(C(=O)NC3(c4ccc(C(=O)O)cc4)CC3)c3c2ccn3Cc2ccc3ccc(F)cc3n2)c1 ; N#Cc1cc(F)cc(Nc2ccc(C(=O)NC3(c4ccc(C(=O)O)cc4)CC3)c3c2ccn3Cc2ccc3ncccc3c2)c1]

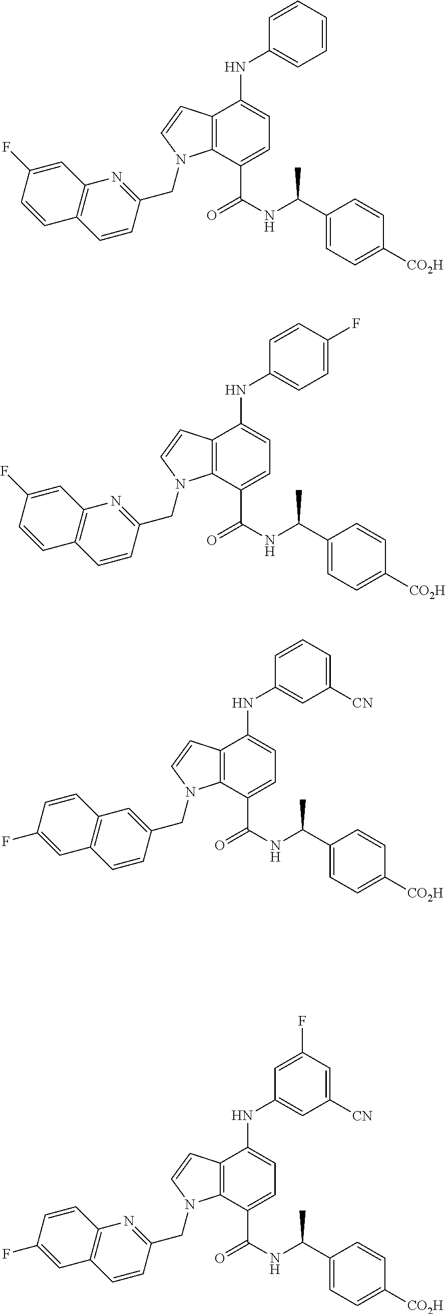 [SMILES: C[C@H](NC(=O)c1ccc(Nc2ccccc2)c2ccn(Cc3ccc4ccc(F)cc4n3)c12)c1ccc(C(=O)O)cc1 ; C[C@H](NC(=O)c1ccc(Nc2ccc(F)cc2)c2ccn(Cc3ccc4ccc(F)cc4n3)c12)c1ccc(C(=O)O)cc1 ; C[C@H](NC(=O)c1ccc(Nc2cccc(C#N)c2)c2ccn(Cc3ccc4cc(F)ccc4c3)c12)c1ccc(C(=O)O)cc1 ; C[C@H](NC(=O)c1ccc(Nc2cc(F)cc(C#N)c2)c2ccn(Cc3ccc4cc(F)ccc4n3)c12)c1ccc(C(=O)O)cc1]

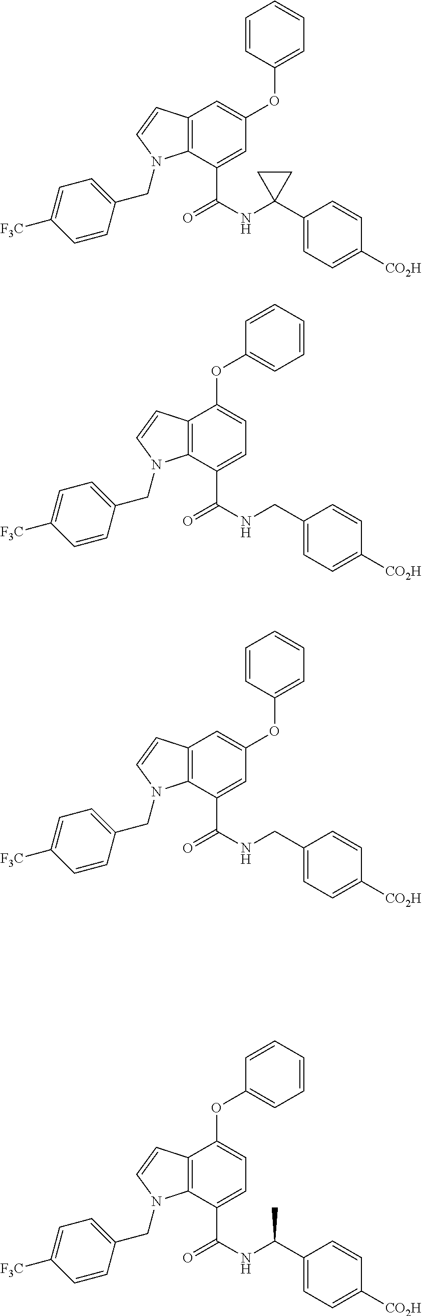 [SMILES: O=C(O)c1ccc(C2(NC(=O)c3cc(Oc4ccccc4)cc4ccn(Cc5ccc(C(F)(F)F)cc5)c34)CC2)cc1 ; O=C(O)c1ccc(CNC(=O)c2ccc(Oc3ccccc3)c3ccn(Cc4ccc(C(F)(F)F)cc4)c23)cc1 ; O=C(O)c1ccc(CNC(=O)c2cc(Oc3ccccc3)cc3ccn(Cc4ccc(C(F)(F)F)cc4)c23)cc1 ; C[C@H](NC(=O)c1ccc(Oc2ccccc2)c2ccn(Cc3ccc(C(F)(F)F)cc3)c12)c1ccc(C(=O)O)cc1]

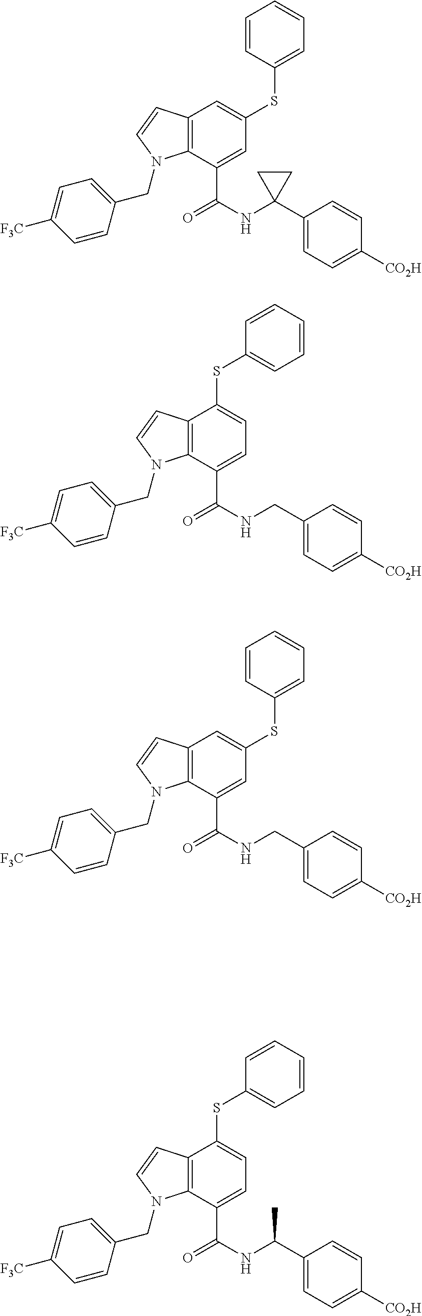 [SMILES: O=C(O)c1ccc(C2(NC(=O)c3cc(Sc4ccccc4)cc4ccn(Cc5ccc(C(F)(F)F)cc5)c34)CC2)cc1 ; O=C(O)c1ccc(CNC(=O)c2ccc(Sc3ccccc3)c3ccn(Cc4ccc(C(F)(F)F)cc4)c23)cc1 ; O=C(O)c1ccc(CNC(=O)c2cc(Sc3ccccc3)cc3ccn(Cc4ccc(C(F)(F)F)cc4)c23)cc1 ; C[C@H](NC(=O)c1ccc(Sc2ccccc2)c2ccn(Cc3ccc(C(F)(F)F)cc3)c12)c1ccc(C(=O)O)cc1]

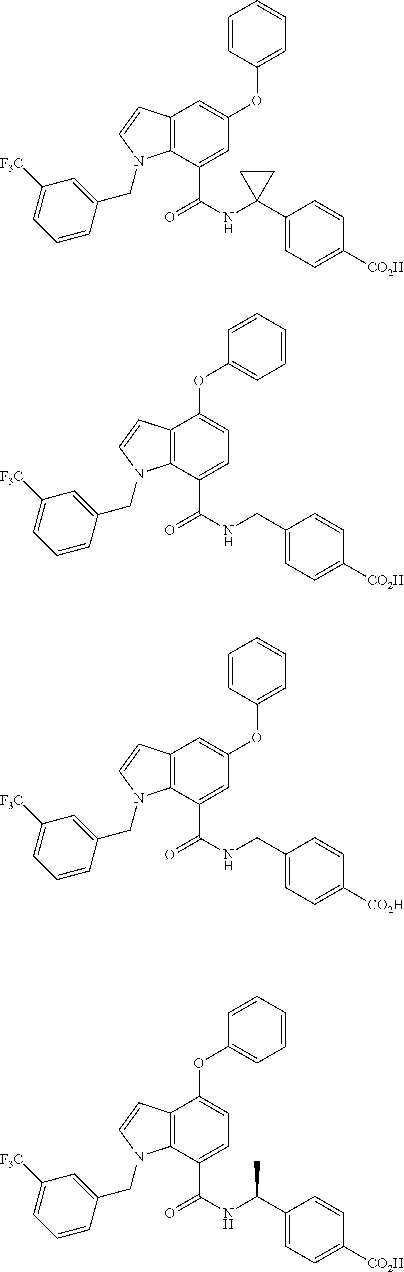 [SMILES: O=C(O)c1ccc(C2(NC(=O)c3cc(Oc4ccccc4)cc4ccn(Cc5cccc(C(F)(F)F)c5)c34)CC2)cc1 ; O=C(O)c1ccc(CNC(=O)c2ccc(Oc3ccccc3)c3ccn(Cc4cccc(C(F)(F)F)c4)c23)cc1 ; O=C(O)c1ccc(CNC(=O)c2cc(Oc3ccccc3)cc3ccn(Cc4cccc(C(F)(F)F)c4)c23)cc1 ; C[C@H](NC(=O)c1ccc(Oc2ccccc2)c2ccn(Cc3cccc(C(F)(F)F)c3)c12)c1ccc(C(=O)O)cc1]

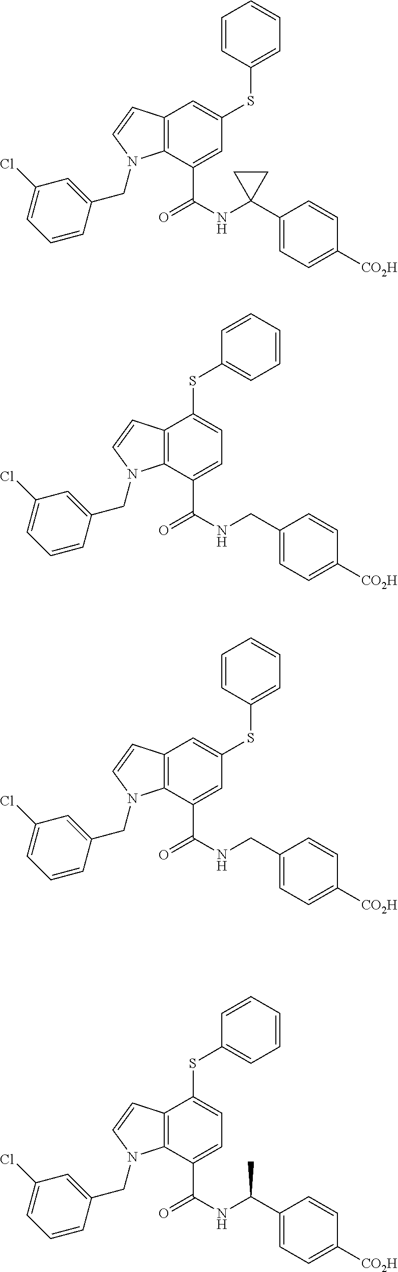 [SMILES: O=C(O)c1ccc(C2(NC(=O)c3cc(Sc4ccccc4)cc4ccn(Cc5cccc(Cl)c5)c34)CC2)cc1 ; O=C(O)c1ccc(CNC(=O)c2ccc(Sc3ccccc3)c3ccn(Cc4cccc(Cl)c4)c23)cc1 ; O=C(O)c1ccc(CNC(=O)c2cc(Sc3ccccc3)cc3ccn(Cc4cccc(Cl)c4)c23)cc1 ; C[C@H](NC(=O)c1ccc(Sc2ccccc2)c2ccn(Cc3cccc(Cl)c3)c12)c1ccc(C(=O)O)cc1]

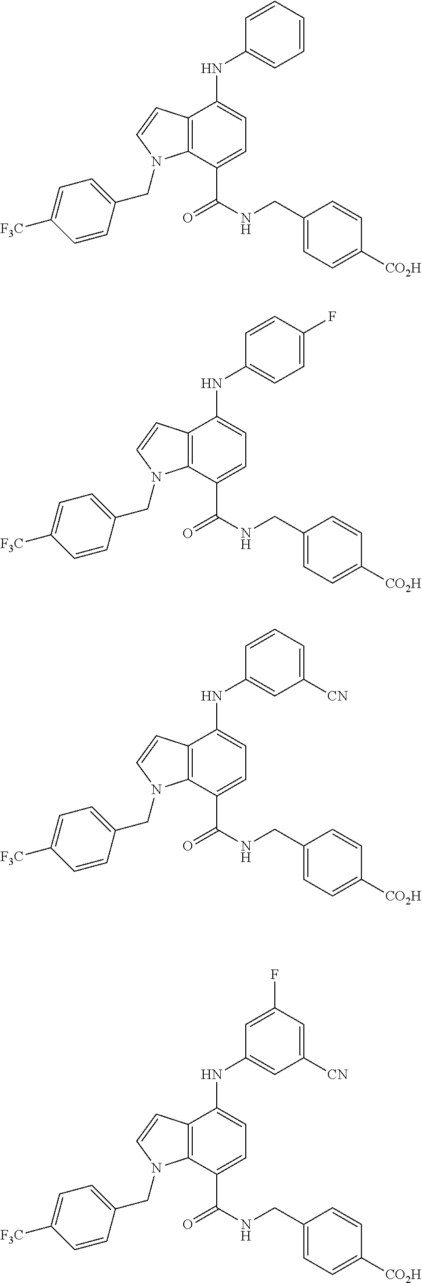 [SMILES: O=C(O)c1ccc(CNC(=O)c2ccc(Nc3ccccc3)c3ccn(Cc4ccc(C(F)(F)F)cc4)c23)cc1 ; O=C(O)c1ccc(CNC(=O)c2ccc(Nc3ccc(F)cc3)c3ccn(Cc4ccc(C(F)(F)F)cc4)c23)cc1 ; N#Cc1cccc(Nc2ccc(C(=O)NCc3ccc(C(=O)O)cc3)c3c2ccn3Cc2ccc(C(F)(F)F)cc2)c1 ; N#Cc1cc(F)cc(Nc2ccc(C(=O)NCc3ccc(C(=O)O)cc3)c3c2ccn3Cc2ccc(C(F)(F)F)cc2)c1]

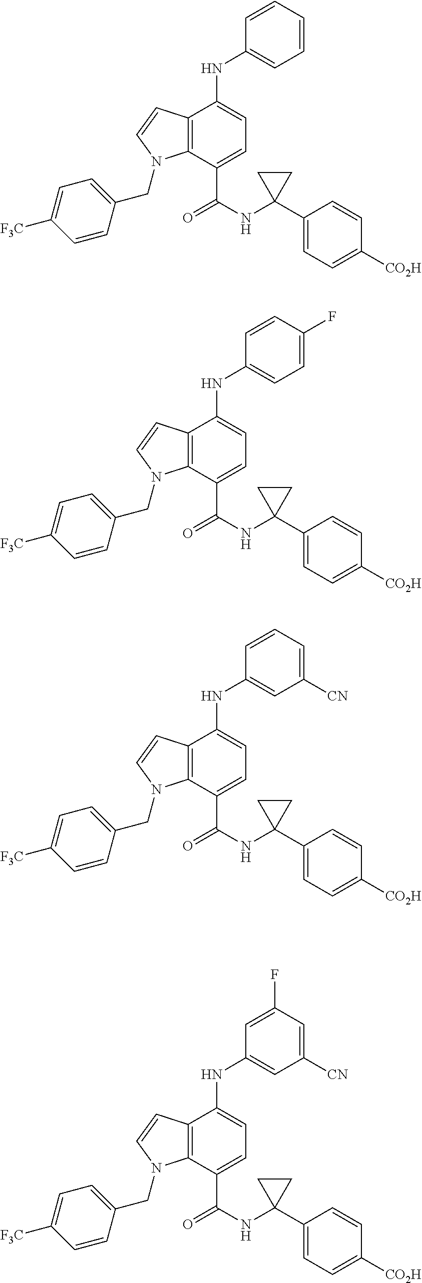 [SMILES: O=C(O)c1ccc(C2(NC(=O)c3ccc(Nc4ccccc4)c4ccn(Cc5ccc(C(F)(F)F)cc5)c34)CC2)cc1 ; O=C(O)c1ccc(C2(NC(=O)c3ccc(Nc4ccc(F)cc4)c4ccn(Cc5ccc(C(F)(F)F)cc5)c34)CC2)cc1 ; N#Cc1cccc(Nc2ccc(C(=O)NC3(c4ccc(C(=O)O)cc4)CC3)c3c2ccn3Cc2ccc(C(F)(F)F)cc2)c1 ; N#Cc1cc(F)cc(Nc2ccc(C(=O)NC3(c4ccc(C(=O)O)cc4)CC3)c3c2ccn3Cc2ccc(C(F)(F)F)cc2)c1]

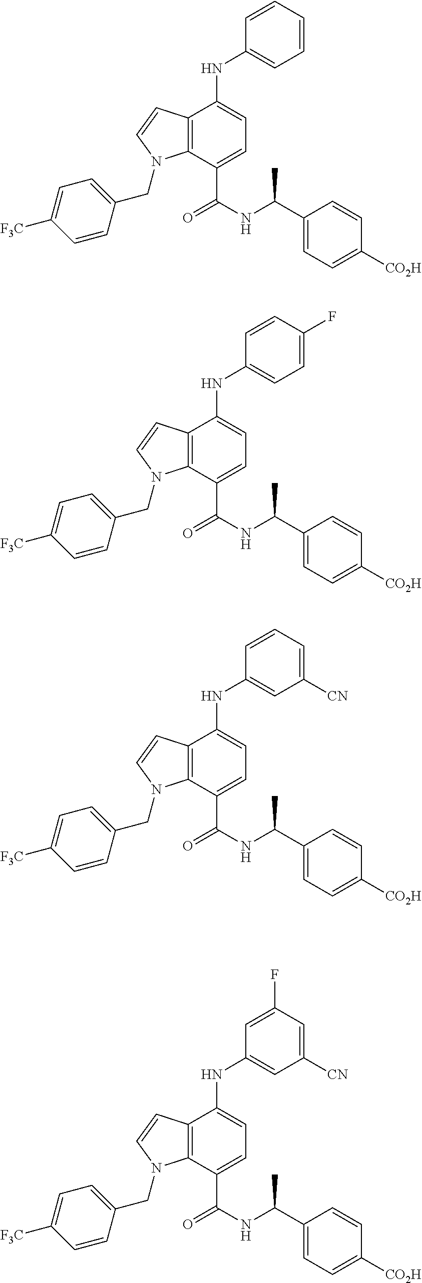 [SMILES: C[C@H](NC(=O)c1ccc(Nc2ccccc2)c2ccn(Cc3ccc(C(F)(F)F)cc3)c12)c1ccc(C(=O)O)cc1 ; C[C@H](NC(=O)c1ccc(Nc2ccc(F)cc2)c2ccn(Cc3ccc(C(F)(F)F)cc3)c12)c1ccc(C(=O)O)cc1 ; C[C@H](NC(=O)c1ccc(Nc2cccc(C#N)c2)c2ccn(Cc3ccc(C(F)(F)F)cc3)c12)c1ccc(C(=O)O)cc1 ; C[C@H](NC(=O)c1ccc(Nc2cc(F)cc(C#N)c2)c2ccn(Cc3ccc(C(F)(F)F)cc3)c12)c1ccc(C(=O)O)cc1]

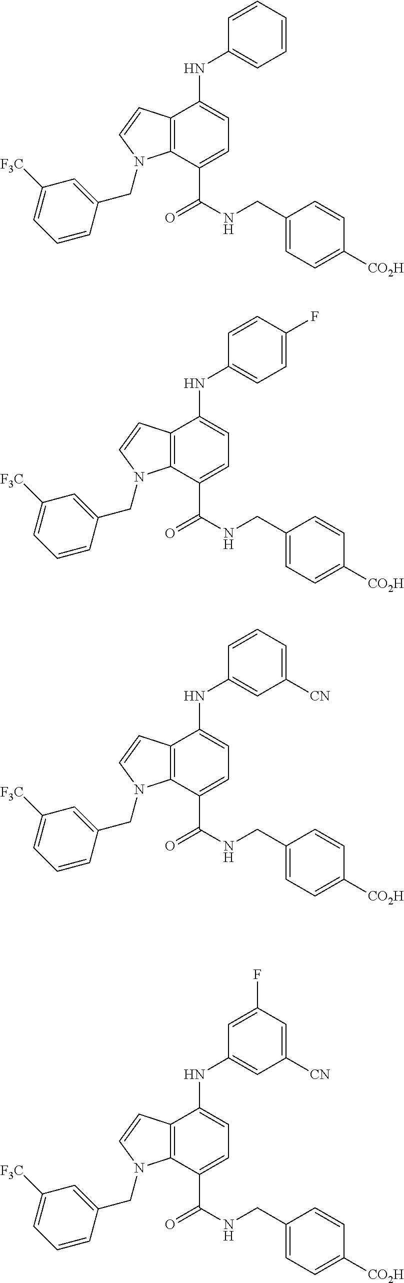 [SMILES: O=C(O)c1ccc(CNC(=O)c2ccc(Nc3ccccc3)c3ccn(Cc4cccc(C(F)(F)F)c4)c23)cc1 ; O=C(O)c1ccc(CNC(=O)c2ccc(Nc3ccc(F)cc3)c3ccn(Cc4cccc(C(F)(F)F)c4)c23)cc1 ; N#Cc1cccc(Nc2ccc(C(=O)NCc3ccc(C(=O)O)cc3)c3c2ccn3Cc2cccc(C(F)(F)F)c2)c1 ; N#Cc1cc(F)cc(Nc2ccc(C(=O)NCc3ccc(C(=O)O)cc3)c3c2ccn3Cc2cccc(C(F)(F)F)c2)c1]

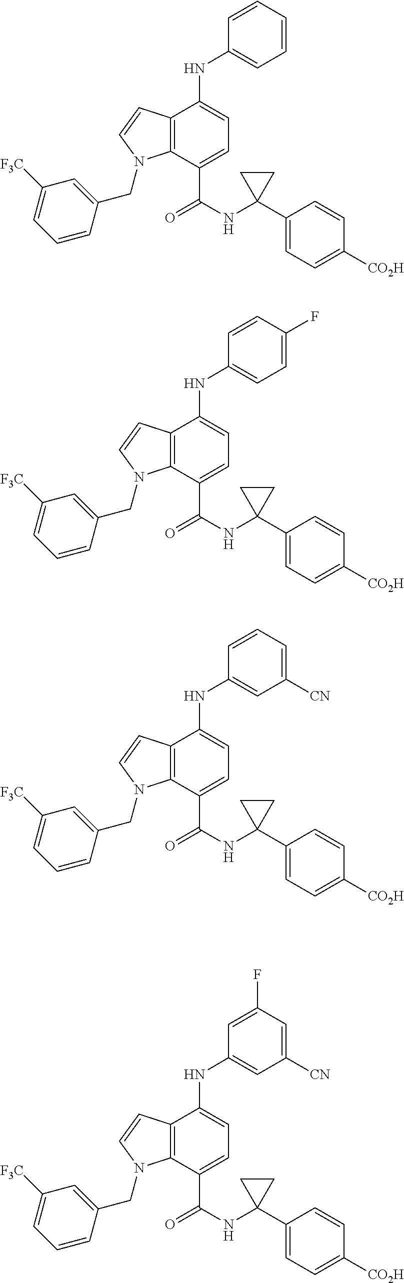 [SMILES: O=C(O)c1ccc(C2(NC(=O)c3ccc(Nc4ccccc4)c4ccn(Cc5cccc(C(F)(F)F)c5)c34)CC2)cc1 ; O=C(O)c1ccc(C2(NC(=O)c3ccc(Nc4ccc(F)cc4)c4ccn(Cc5cccc(C(F)(F)F)c5)c34)CC2)cc1 ; N#Cc1cccc(Nc2ccc(C(=O)NC3(c4ccc(C(=O)O)cc4)CC3)c3c2ccn3Cc2cccc(C(F)(F)F)c2)c1 ; N#Cc1cc(F)cc(Nc2ccc(C(=O)NC3(c4ccc(C(=O)O)cc4)CC3)c3c2ccn3Cc2cccc(C(F)(F)F)c2)c1]

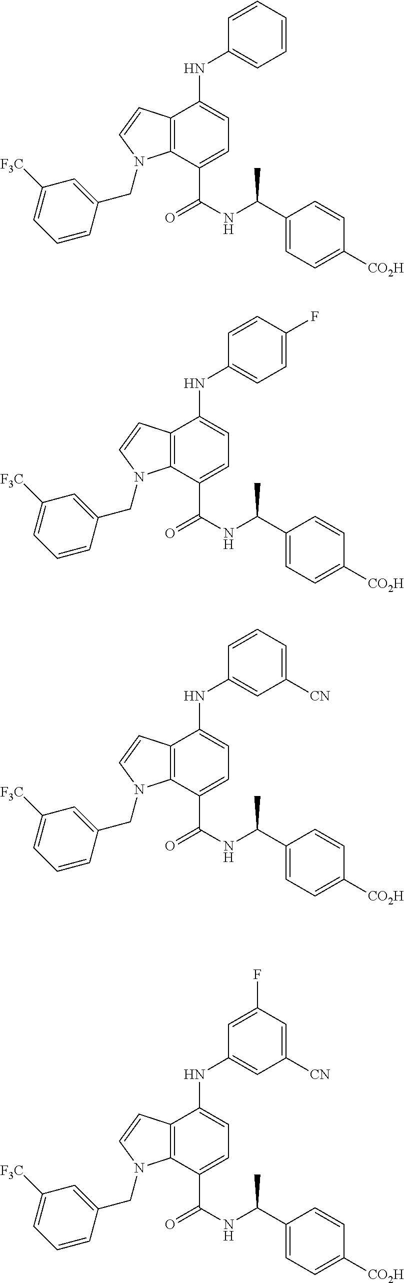 [SMILES: C[C@H](NC(=O)c1ccc(Nc2ccccc2)c2ccn(Cc3cccc(C(F)(F)F)c3)c12)c1ccc(C(=O)O)cc1 ; C[C@H](NC(=O)c1ccc(Nc2ccc(F)cc2)c2ccn(Cc3cccc(C(F)(F)F)c3)c12)c1ccc(C(=O)O)cc1 ; C[C@H](NC(=O)c1ccc(Nc2cccc(C#N)c2)c2ccn(Cc3cccc(C(F)(F)F)c3)c12)c1ccc(C(=O)O)cc1 ; C[C@H](NC(=O)c1ccc(Nc2cc(F)cc(C#N)c2)c2ccn(Cc3cccc(C(F)(F)F)c3)c12)c1ccc(C(=O)O)cc1]

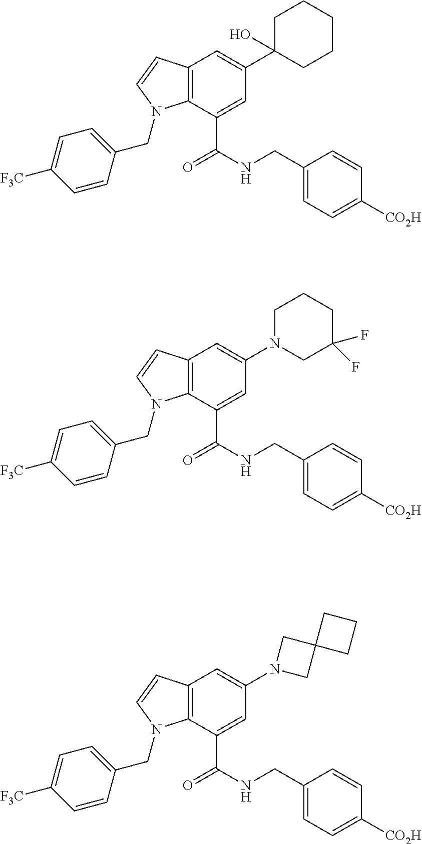 [SMILES: O=C(O)c1ccc(CNC(=O)c2cc(C3(O)CCCCC3)cc3ccn(Cc4ccc(C(F)(F)F)cc4)c23)cc1 ; O=C(O)c1ccc(CNC(=O)c2cc(N3CCCC(F)(F)C3)cc3ccn(Cc4ccc(C(F)(F)F)cc4)c23)cc1 ; O=C(O)c1ccc(CNC(=O)c2cc(N3CC4(CCC4)C3)cc3ccn(Cc4ccc(C(F)(F)F)cc4)c23)cc1]

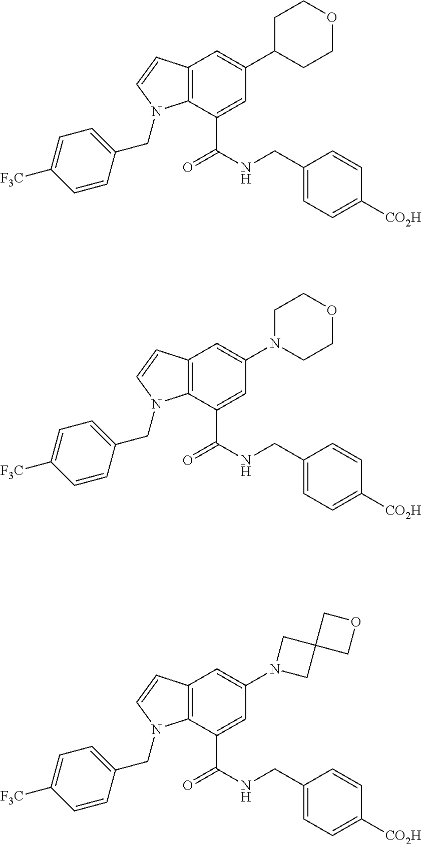 [SMILES: O=C(O)c1ccc(CNC(=O)c2cc(C3CCOCC3)cc3ccn(Cc4ccc(C(F)(F)F)cc4)c23)cc1 ; O=C(O)c1ccc(CNC(=O)c2cc(N3CCOCC3)cc3ccn(Cc4ccc(C(F)(F)F)cc4)c23)cc1 ; O=C(O)c1ccc(CNC(=O)c2cc(N3CC4(COC4)C3)cc3ccn(Cc4ccc(C(F)(F)F)cc4)c23)cc1]

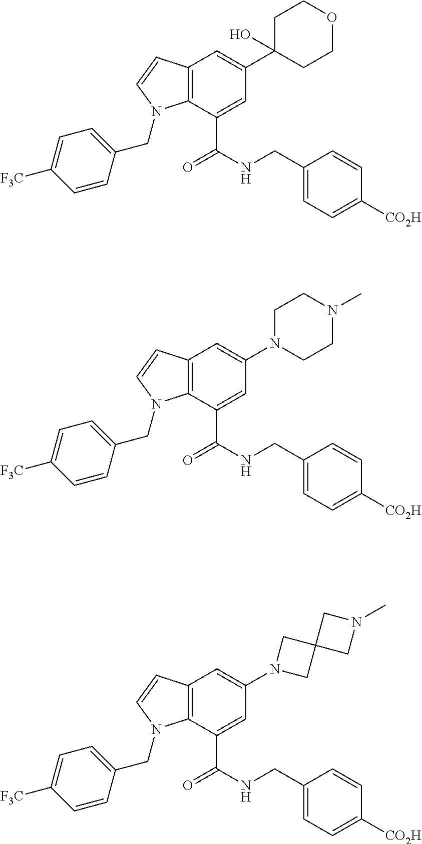 [SMILES: O=C(O)c1ccc(CNC(=O)c2cc(C3(O)CCOCC3)cc3ccn(Cc4ccc(C(F)(F)F)cc4)c23)cc1 ; CN1CCN(c2cc(C(=O)NCc3ccc(C(=O)O)cc3)c3c(ccn3Cc3ccc(C(F)(F)F)cc3)c2)CC1 ; CN1CC2(C1)CN(c1cc(C(=O)NCc3ccc(C(=O)O)cc3)c3c(ccn3Cc3ccc(C(F)(F)F)cc3)c1)C2]

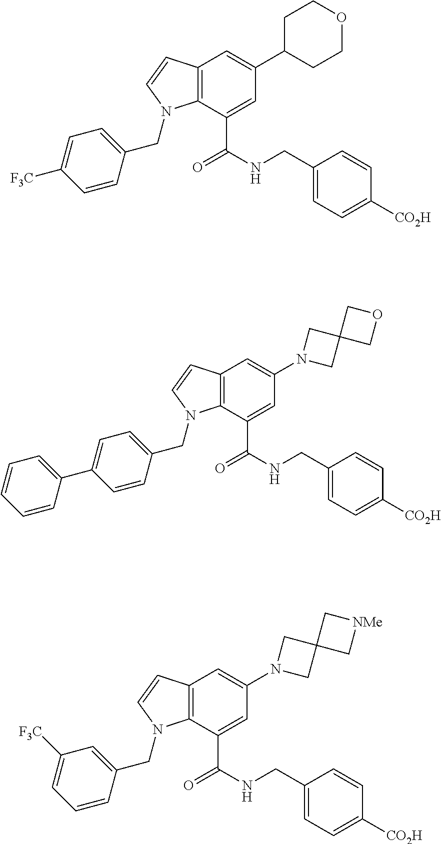 [SMILES: O=C(O)c1ccc(CNC(=O)c2cc(C3CCOCC3)cc3ccn(Cc4ccc(C(F)(F)F)cc4)c23)cc1 ; O=C(O)c1ccc(CNC(=O)c2cc(N3CC4(COC4)C3)cc3ccn(Cc4ccc(-c5ccccc5)cc4)c23)cc1 ; CN1CC2(C1)CN(c1cc(C(=O)NCc3ccc(C(=O)O)cc3)c3c(ccn3Cc3cccc(C(F)(F)F)c3)c1)C2]

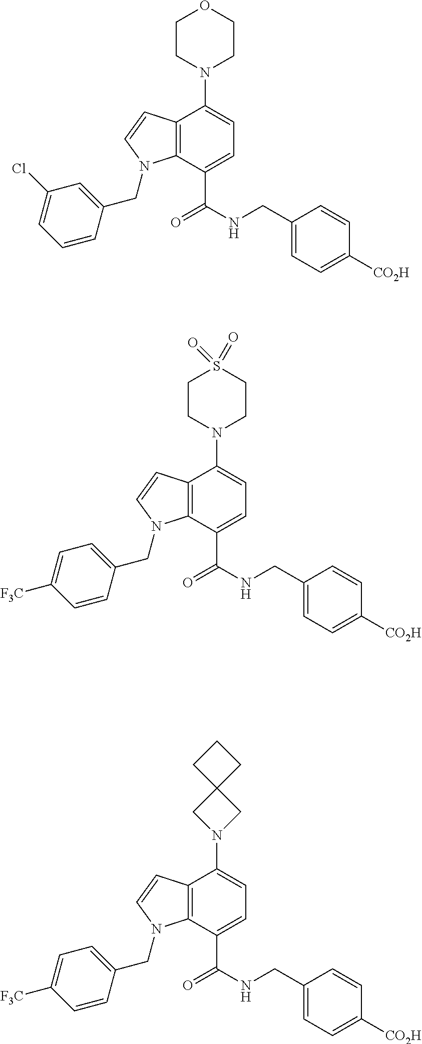 [SMILES: O=C(O)c1ccc(CNC(=O)c2ccc(N3CCOCC3)c3ccn(Cc4cccc(Cl)c4)c23)cc1 ; O=C(O)c1ccc(CNC(=O)c2ccc(N3CCS(=O)(=O)CC3)c3ccn(Cc4ccc(C(F)(F)F)cc4)c23)cc1 ; O=C(O)c1ccc(CNC(=O)c2ccc(N3CC4(CCC4)C3)c3ccn(Cc4ccc(C(F)(F)F)cc4)c23)cc1]

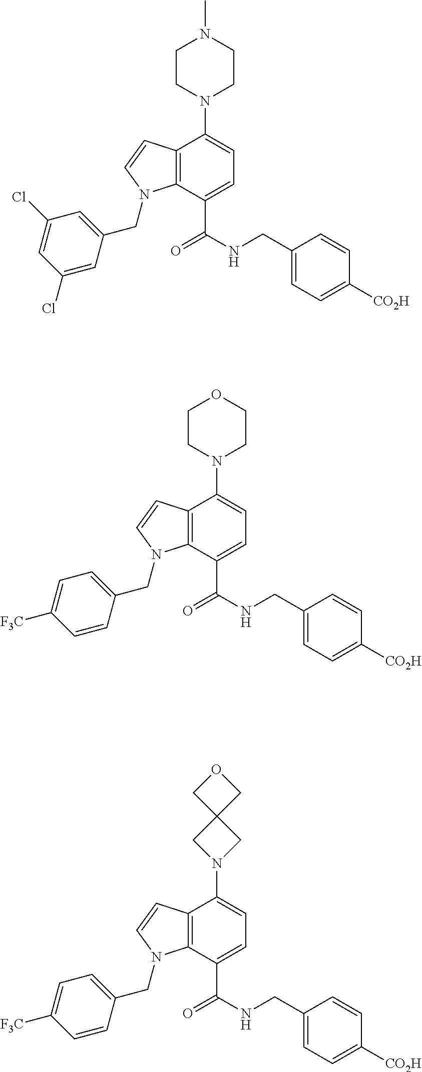 [SMILES: CN1CCN(c2ccc(C(=O)NCc3ccc(C(=O)O)cc3)c3c2ccn3Cc2cc(Cl)cc(Cl)c2)CC1 ; O=C(O)c1ccc(CNC(=O)c2ccc(N3CCOCC3)c3ccn(Cc4ccc(C(F)(F)F)cc4)c23)cc1 ; O=C(O)c1ccc(CNC(=O)c2ccc(N3CC4(COC4)C3)c3ccn(Cc4ccc(C(F)(F)F)cc4)c23)cc1]

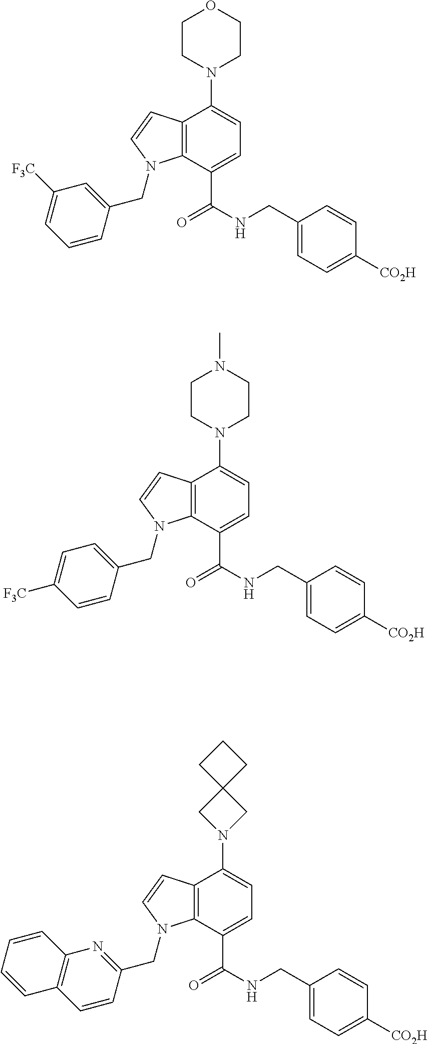 [SMILES: O=C(O)c1ccc(CNC(=O)c2ccc(N3CCOCC3)c3ccn(Cc4cccc(C(F)(F)F)c4)c23)cc1 ; CN1CCN(c2ccc(C(=O)NCc3ccc(C(=O)O)cc3)c3c2ccn3Cc2ccc(C(F)(F)F)cc2)CC1 ; O=C(O)c1ccc(CNC(=O)c2ccc(N3CC4(CCC4)C3)c3ccn(Cc4ccc5ccccc5n4)c23)cc1]

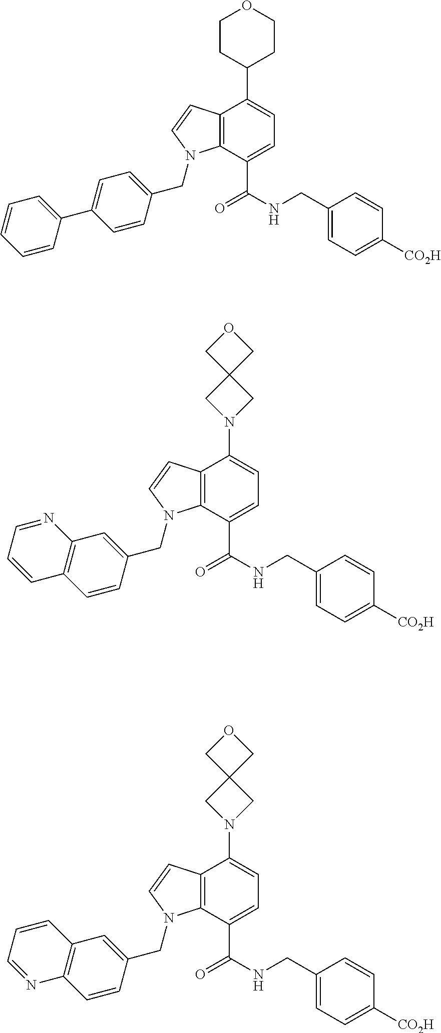 [SMILES: O=C(O)c1ccc(CNC(=O)c2ccc(C3CCOCC3)c3ccn(Cc4ccc(-c5ccccc5)cc4)c23)cc1 ; O=C(O)c1ccc(CNC(=O)c2ccc(N3CC4(COC4)C3)c3ccn(Cc4ccc5cccnc5c4)c23)cc1 ; O=C(O)c1ccc(CNC(=O)c2ccc(N3CC4(COC4)C3)c3ccn(Cc4ccc5ncccc5c4)c23)cc1]

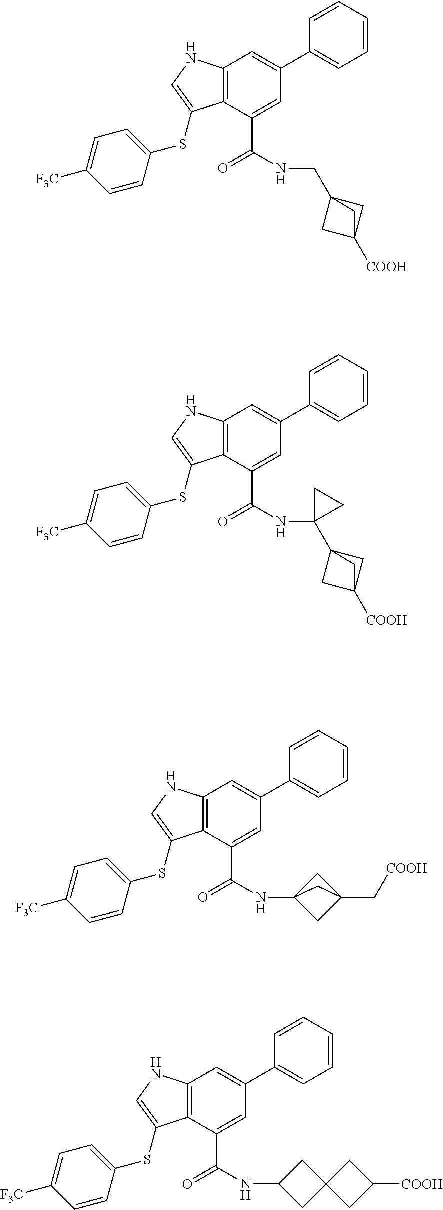 [SMILES: O=C(NCC12CC(C(=O)O)(C1)C2)c1cc(-c2ccccc2)cc2[nH]cc(Sc3ccc(C(F)(F)F)cc3)c12 ; O=C(NC1(C23CC(C(=O)O)(C2)C3)CC1)c1cc(-c2ccccc2)cc2[nH]cc(Sc3ccc(C(F)(F)F)cc3)c12 ; O=C(O)CC12CC(NC(=O)c3cc(-c4ccccc4)cc4[nH]cc(Sc5ccc(C(F)(F)F)cc5)c34)(C1)C2 ; O=C(NC1CC2(C1)CC(C(=O)O)C2)c1cc(-c2ccccc2)cc2[nH]cc(Sc3ccc(C(F)(F)F)cc3)c12]

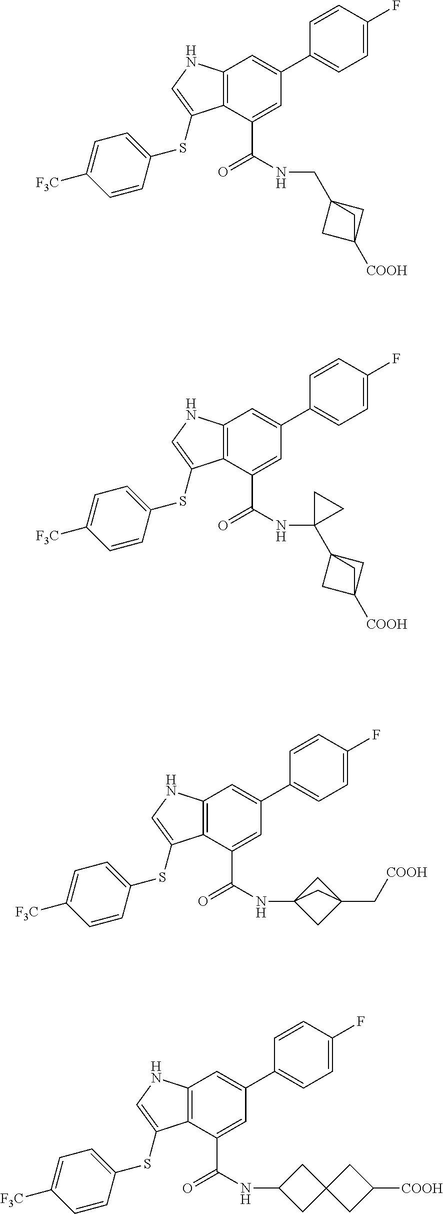 [SMILES: O=C(NCC12CC(C(=O)O)(C1)C2)c1cc(-c2ccc(F)cc2)cc2[nH]cc(Sc3ccc(C(F)(F)F)cc3)c12 ; O=C(NC1(C23CC(C(=O)O)(C2)C3)CC1)c1cc(-c2ccc(F)cc2)cc2[nH]cc(Sc3ccc(C(F)(F)F)cc3)c12 ; O=C(O)CC12CC(NC(=O)c3cc(-c4ccc(F)cc4)cc4[nH]cc(Sc5ccc(C(F)(F)F)cc5)c34)(C1)C2 ; O=C(NC1CC2(C1)CC(C(=O)O)C2)c1cc(-c2ccc(F)cc2)cc2[nH]cc(Sc3ccc(C(F)(F)F)cc3)c12]

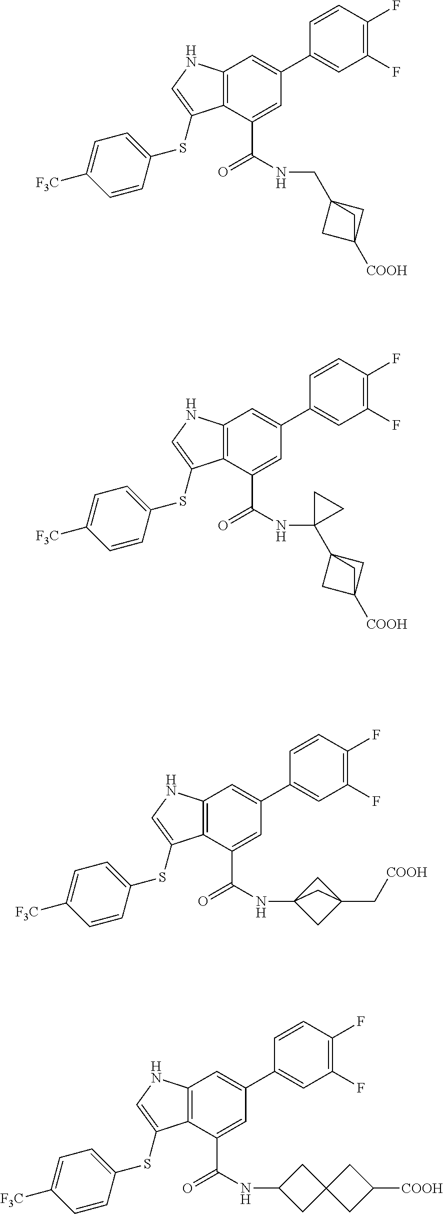 [SMILES: O=C(NCC12CC(C(=O)O)(C1)C2)c1cc(-c2ccc(F)c(F)c2)cc2[nH]cc(Sc3ccc(C(F)(F)F)cc3)c12 ; O=C(NC1(C23CC(C(=O)O)(C2)C3)CC1)c1cc(-c2ccc(F)c(F)c2)cc2[nH]cc(Sc3ccc(C(F)(F)F)cc3)c12 ; O=C(O)CC12CC(NC(=O)c3cc(-c4ccc(F)c(F)c4)cc4[nH]cc(Sc5ccc(C(F)(F)F)cc5)c34)(C1)C2 ; O=C(NC1CC2(C1)CC(C(=O)O)C2)c1cc(-c2ccc(F)c(F)c2)cc2[nH]cc(Sc3ccc(C(F)(F)F)cc3)c12]

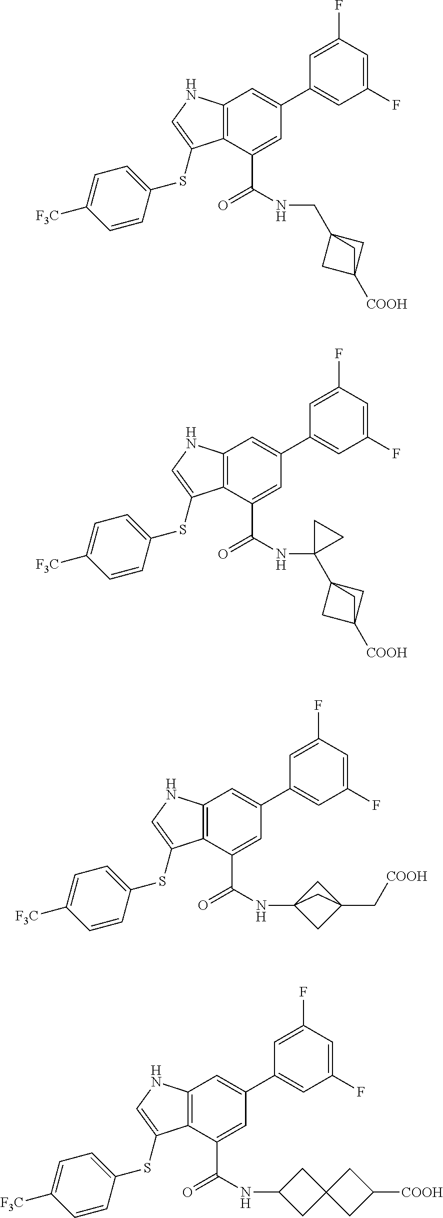 [SMILES: O=C(NCC12CC(C(=O)O)(C1)C2)c1cc(-c2cc(F)cc(F)c2)cc2[nH]cc(Sc3ccc(C(F)(F)F)cc3)c12 ; O=C(NC1(C23CC(C(=O)O)(C2)C3)CC1)c1cc(-c2cc(F)cc(F)c2)cc2[nH]cc(Sc3ccc(C(F)(F)F)cc3)c12 ; O=C(O)CC12CC(NC(=O)c3cc(-c4cc(F)cc(F)c4)cc4[nH]cc(Sc5ccc(C(F)(F)F)cc5)c34)(C1)C2 ; O=C(NC1CC2(C1)CC(C(=O)O)C2)c1cc(-c2cc(F)cc(F)c2)cc2[nH]cc(Sc3ccc(C(F)(F)F)cc3)c12]

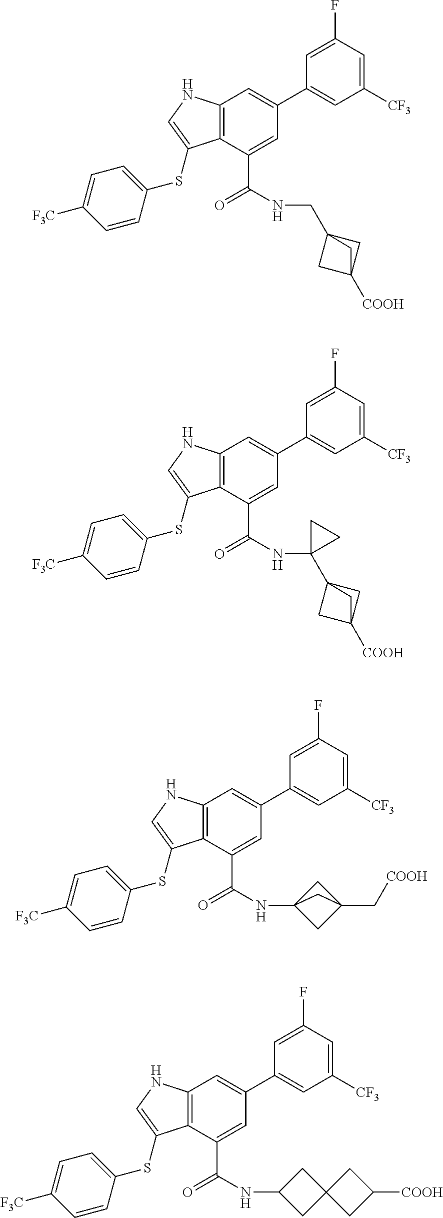 [SMILES: O=C(NCC12CC(C(=O)O)(C1)C2)c1cc(-c2cc(F)cc(C(F)(F)F)c2)cc2[nH]cc(Sc3ccc(C(F)(F)F)cc3)c12 ; O=C(NC1(C23CC(C(=O)O)(C2)C3)CC1)c1cc(-c2cc(F)cc(C(F)(F)F)c2)cc2[nH]cc(Sc3ccc(C(F)(F)F)cc3)c12 ; O=C(O)CC12CC(NC(=O)c3cc(-c4cc(F)cc(C(F)(F)F)c4)cc4[nH]cc(Sc5ccc(C(F)(F)F)cc5)c34)(C1)C2 ; O=C(NC1CC2(C1)CC(C(=O)O)C2)c1cc(-c2cc(F)cc(C(F)(F)F)c2)cc2[nH]cc(Sc3ccc(C(F)(F)F)cc3)c12]

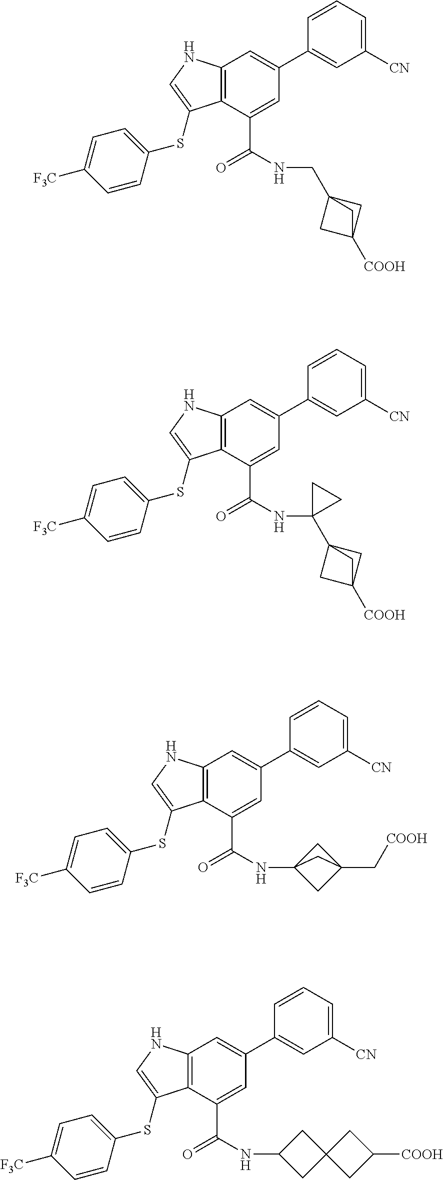 [SMILES: N#Cc1cccc(-c2cc(C(=O)NCC34CC(C(=O)O)(C3)C4)c3c(Sc4ccc(C(F)(F)F)cc4)c[nH]c3c2)c1 ; N#Cc1cccc(-c2cc(C(=O)NC3(C45CC(C(=O)O)(C4)C5)CC3)c3c(Sc4ccc(C(F)(F)F)cc4)c[nH]c3c2)c1 ; N#Cc1cccc(-c2cc(C(=O)NC34CC(CC(=O)O)(C3)C4)c3c(Sc4ccc(C(F)(F)F)cc4)c[nH]c3c2)c1 ; N#Cc1cccc(-c2cc(C(=O)NC3CC4(C3)CC(C(=O)O)C4)c3c(Sc4ccc(C(F)(F)F)cc4)c[nH]c3c2)c1]

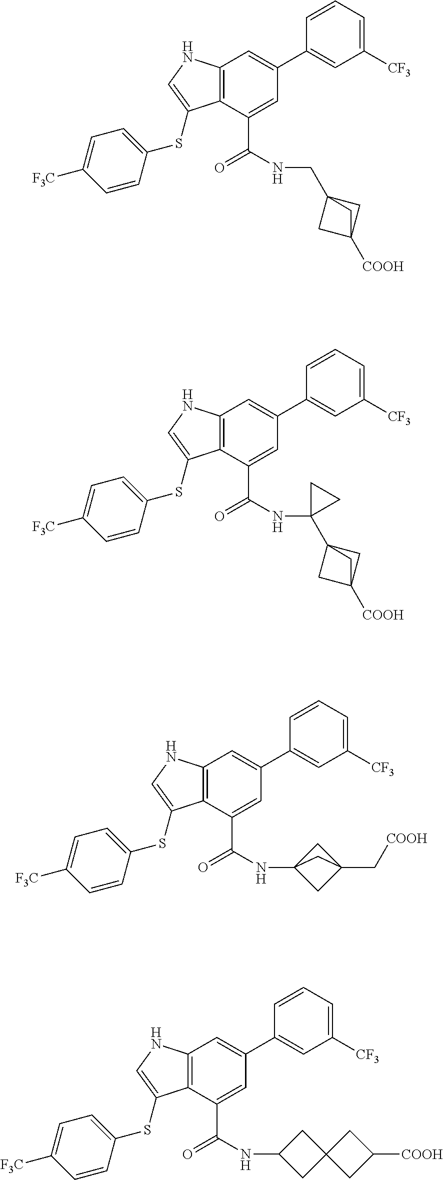 [SMILES: O=C(NCC12CC(C(=O)O)(C1)C2)c1cc(-c2cccc(C(F)(F)F)c2)cc2[nH]cc(Sc3ccc(C(F)(F)F)cc3)c12 ; O=C(NC1(C23CC(C(=O)O)(C2)C3)CC1)c1cc(-c2cccc(C(F)(F)F)c2)cc2[nH]cc(Sc3ccc(C(F)(F)F)cc3)c12 ; O=C(O)CC12CC(NC(=O)c3cc(-c4cccc(C(F)(F)F)c4)cc4[nH]cc(Sc5ccc(C(F)(F)F)cc5)c34)(C1)C2 ; O=C(NC1CC2(C1)CC(C(=O)O)C2)c1cc(-c2cccc(C(F)(F)F)c2)cc2[nH]cc(Sc3ccc(C(F)(F)F)cc3)c12]

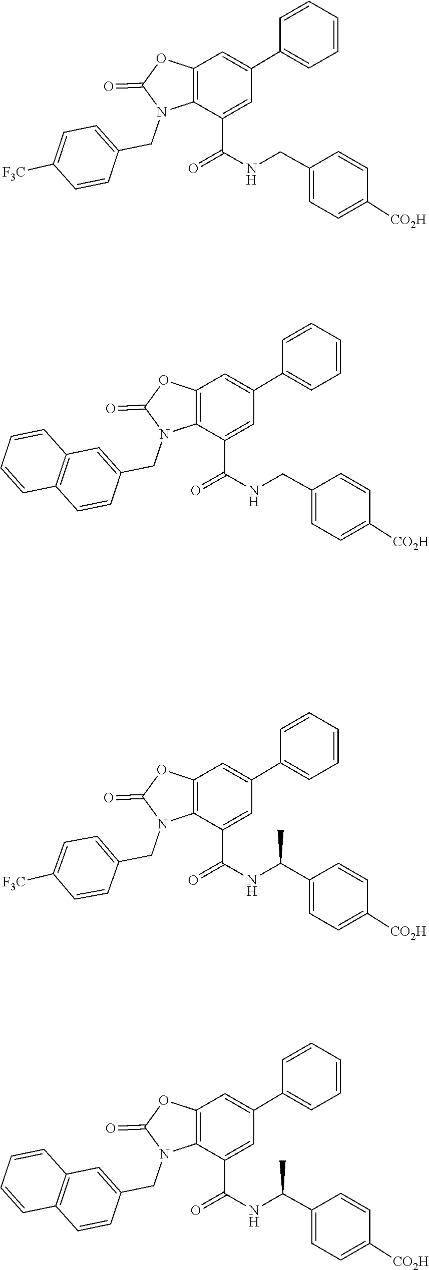 [SMILES: O=C(O)c1ccc(CNC(=O)c2cc(-c3ccccc3)cc3oc(=O)n(Cc4ccc(C(F)(F)F)cc4)c23)cc1 ; O=C(O)c1ccc(CNC(=O)c2cc(-c3ccccc3)cc3oc(=O)n(Cc4ccc5ccccc5c4)c23)cc1 ; C[C@H](NC(=O)c1cc(-c2ccccc2)cc2oc(=O)n(Cc3ccc(C(F)(F)F)cc3)c12)c1ccc(C(=O)O)cc1 ; C[C@H](NC(=O)c1cc(-c2ccccc2)cc2oc(=O)n(Cc3ccc4ccccc4c3)c12)c1ccc(C(=O)O)cc1]

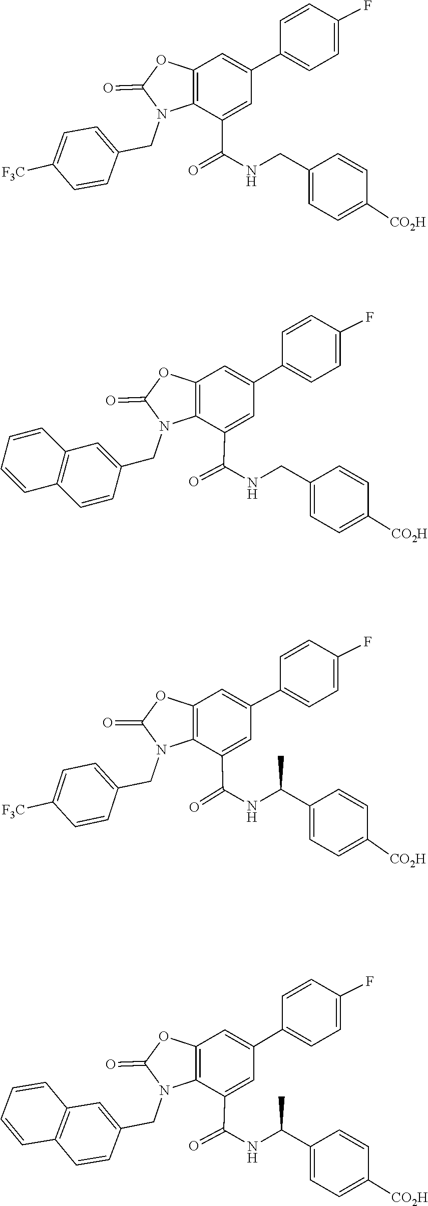 [SMILES: O=C(O)c1ccc(CNC(=O)c2cc(-c3ccc(F)cc3)cc3oc(=O)n(Cc4ccc(C(F)(F)F)cc4)c23)cc1 ; O=C(O)c1ccc(CNC(=O)c2cc(-c3ccc(F)cc3)cc3oc(=O)n(Cc4ccc5ccccc5c4)c23)cc1 ; C[C@H](NC(=O)c1cc(-c2ccc(F)cc2)cc2oc(=O)n(Cc3ccc(C(F)(F)F)cc3)c12)c1ccc(C(=O)O)cc1 ; C[C@H](NC(=O)c1cc(-c2ccc(F)cc2)cc2oc(=O)n(Cc3ccc4ccccc4c3)c12)c1ccc(C(=O)O)cc1]

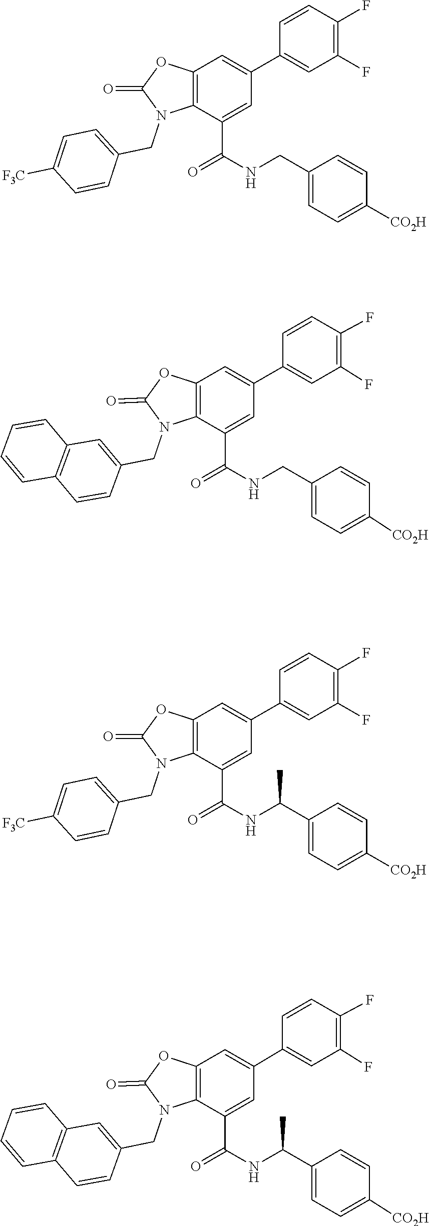 [SMILES: O=C(O)c1ccc(CNC(=O)c2cc(-c3ccc(F)c(F)c3)cc3oc(=O)n(Cc4ccc(C(F)(F)F)cc4)c23)cc1 ; O=C(O)c1ccc(CNC(=O)c2cc(-c3ccc(F)c(F)c3)cc3oc(=O)n(Cc4ccc5ccccc5c4)c23)cc1 ; C[C@H](NC(=O)c1cc(-c2ccc(F)c(F)c2)cc2oc(=O)n(Cc3ccc(C(F)(F)F)cc3)c12)c1ccc(C(=O)O)cc1 ; C[C@H](NC(=O)c1cc(-c2ccc(F)c(F)c2)cc2oc(=O)n(Cc3ccc4ccccc4c3)c12)c1ccc(C(=O)O)cc1]

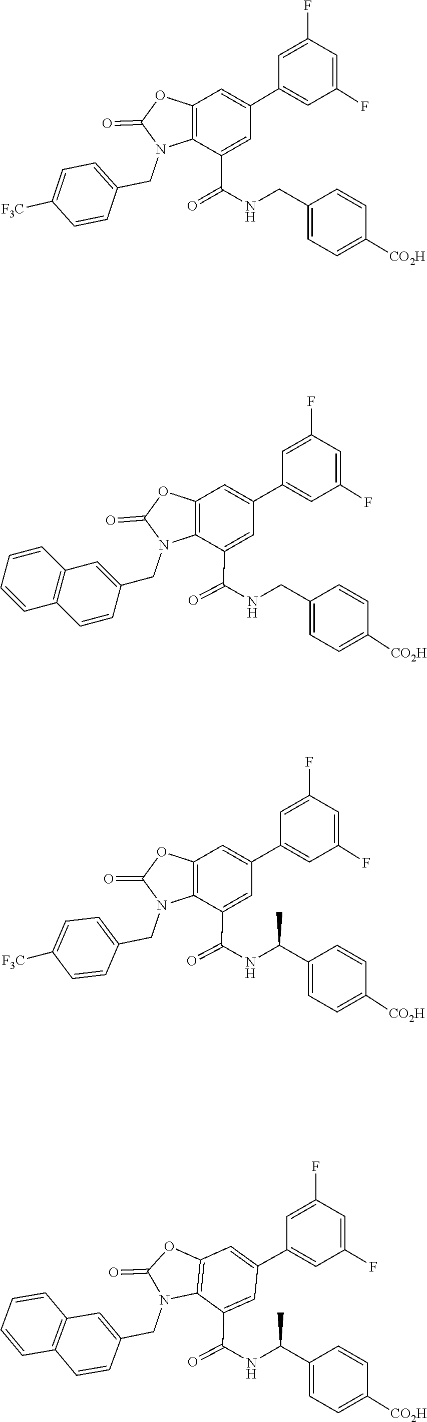 [SMILES: O=C(O)c1ccc(CNC(=O)c2cc(-c3cc(F)cc(F)c3)cc3oc(=O)n(Cc4ccc(C(F)(F)F)cc4)c23)cc1 ; O=C(O)c1ccc(CNC(=O)c2cc(-c3cc(F)cc(F)c3)cc3oc(=O)n(Cc4ccc5ccccc5c4)c23)cc1 ; C[C@H](NC(=O)c1cc(-c2cc(F)cc(F)c2)cc2oc(=O)n(Cc3ccc(C(F)(F)F)cc3)c12)c1ccc(C(=O)O)cc1 ; C[C@H](NC(=O)c1cc(-c2cc(F)cc(F)c2)cc2oc(=O)n(Cc3ccc4ccccc4c3)c12)c1ccc(C(=O)O)cc1]

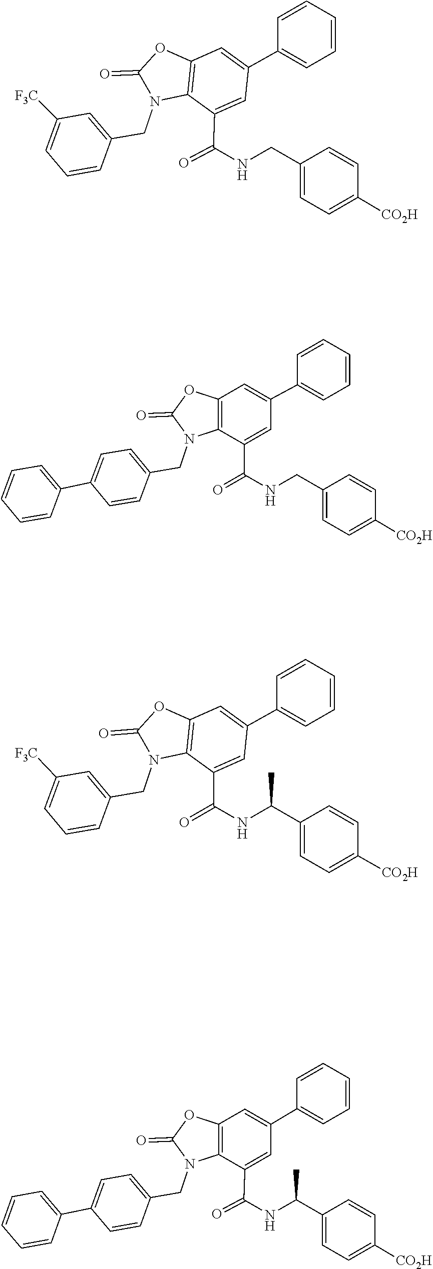 [SMILES: O=C(O)c1ccc(CNC(=O)c2cc(-c3ccccc3)cc3oc(=O)n(Cc4cccc(C(F)(F)F)c4)c23)cc1 ; O=C(O)c1ccc(CNC(=O)c2cc(-c3ccccc3)cc3oc(=O)n(Cc4ccc(-c5ccccc5)cc4)c23)cc1 ; C[C@H](NC(=O)c1cc(-c2ccccc2)cc2oc(=O)n(Cc3cccc(C(F)(F)F)c3)c12)c1ccc(C(=O)O)cc1 ; C[C@H](NC(=O)c1cc(-c2ccccc2)cc2oc(=O)n(Cc3ccc(-c4ccccc4)cc3)c12)c1ccc(C(=O)O)cc1]

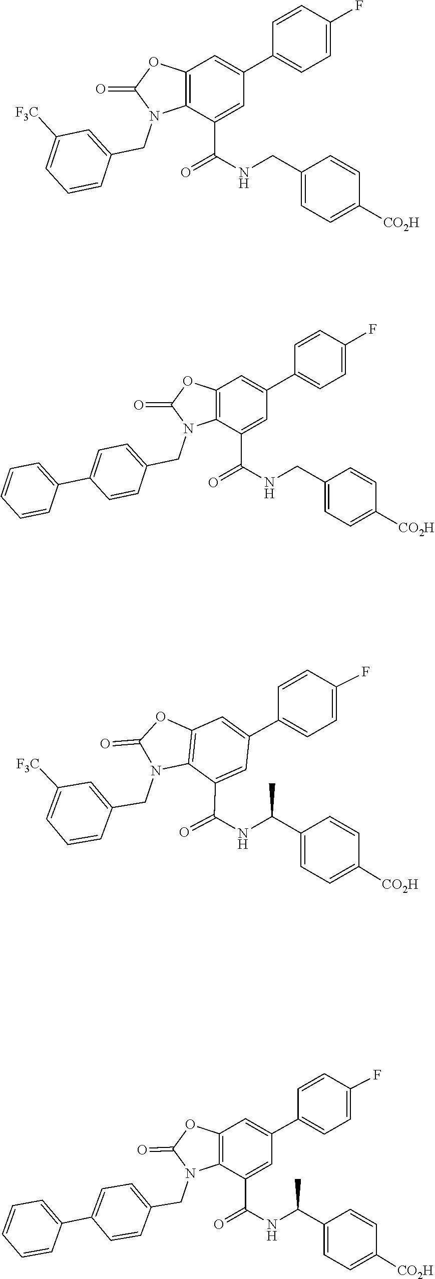 [SMILES: O=C(O)c1ccc(CNC(=O)c2cc(-c3ccc(F)cc3)cc3oc(=O)n(Cc4cccc(C(F)(F)F)c4)c23)cc1 ; O=C(O)c1ccc(CNC(=O)c2cc(-c3ccc(F)cc3)cc3oc(=O)n(Cc4ccc(-c5ccccc5)cc4)c23)cc1 ; C[C@H](NC(=O)c1cc(-c2ccc(F)cc2)cc2oc(=O)n(Cc3cccc(C(F)(F)F)c3)c12)c1ccc(C(=O)O)cc1 ; C[C@H](NC(=O)c1cc(-c2ccc(F)cc2)cc2oc(=O)n(Cc3ccc(-c4ccccc4)cc3)c12)c1ccc(C(=O)O)cc1]

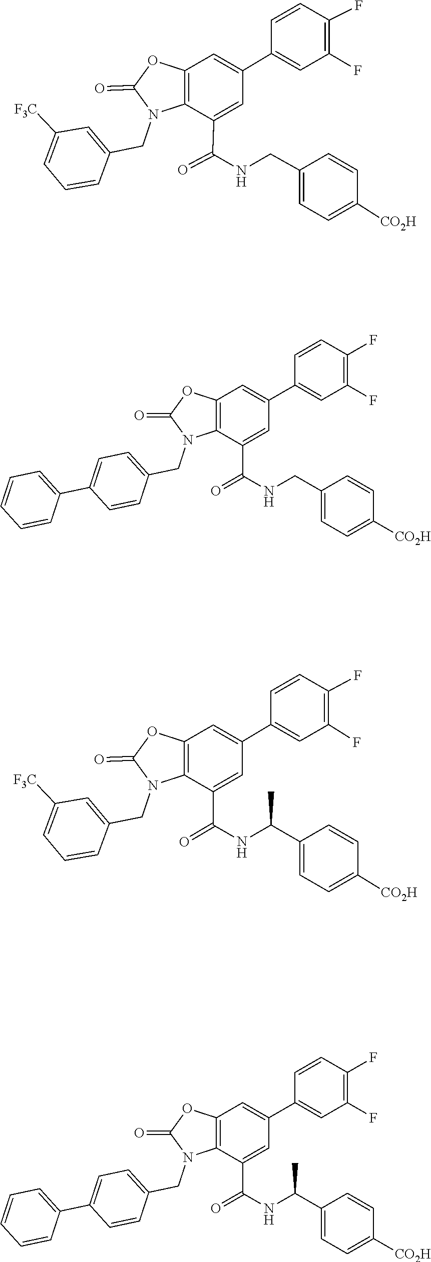 [SMILES: O=C(O)c1ccc(CNC(=O)c2cc(-c3ccc(F)c(F)c3)cc3oc(=O)n(Cc4cccc(C(F)(F)F)c4)c23)cc1 ; O=C(O)c1ccc(CNC(=O)c2cc(-c3ccc(F)c(F)c3)cc3oc(=O)n(Cc4ccc(-c5ccccc5)cc4)c23)cc1 ; C[C@H](NC(=O)c1cc(-c2ccc(F)c(F)c2)cc2oc(=O)n(Cc3cccc(C(F)(F)F)c3)c12)c1ccc(C(=O)O)cc1 ; C[C@H](NC(=O)c1cc(-c2ccc(F)c(F)c2)cc2oc(=O)n(Cc3ccc(-c4ccccc4)cc3)c12)c1ccc(C(=O)O)cc1]

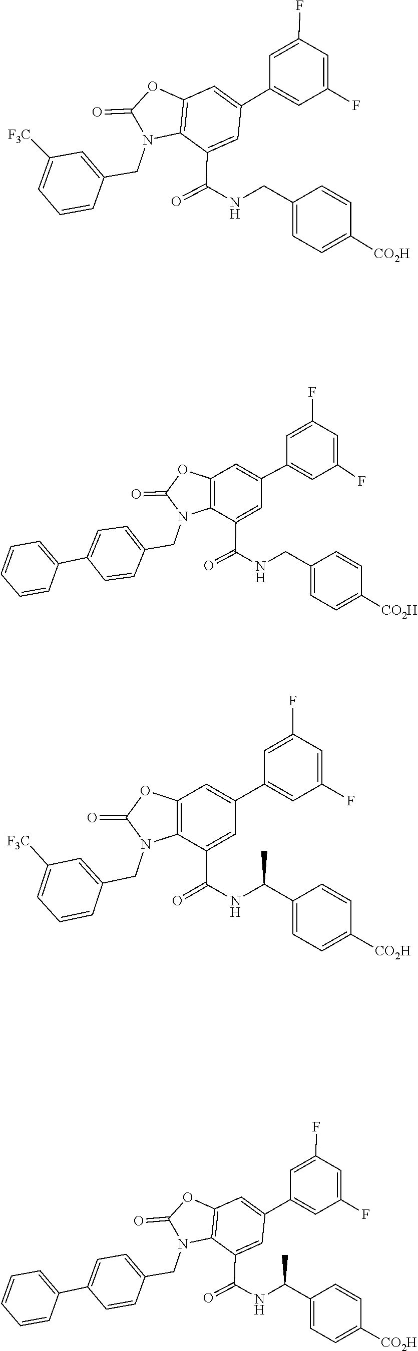 [SMILES: O=C(O)c1ccc(CNC(=O)c2cc(-c3cc(F)cc(F)c3)cc3oc(=O)n(Cc4cccc(C(F)(F)F)c4)c23)cc1 ; O=C(O)c1ccc(CNC(=O)c2cc(-c3cc(F)cc(F)c3)cc3oc(=O)n(Cc4ccc(-c5ccccc5)cc4)c23)cc1 ; C[C@H](NC(=O)c1cc(-c2cc(F)cc(F)c2)cc2oc(=O)n(Cc3cccc(C(F)(F)F)c3)c12)c1ccc(C(=O)O)cc1 ; C[C@H](NC(=O)c1cc(-c2cc(F)cc(F)c2)cc2oc(=O)n(Cc3ccc(-c4ccccc4)cc3)c12)c1ccc(C(=O)O)cc1]

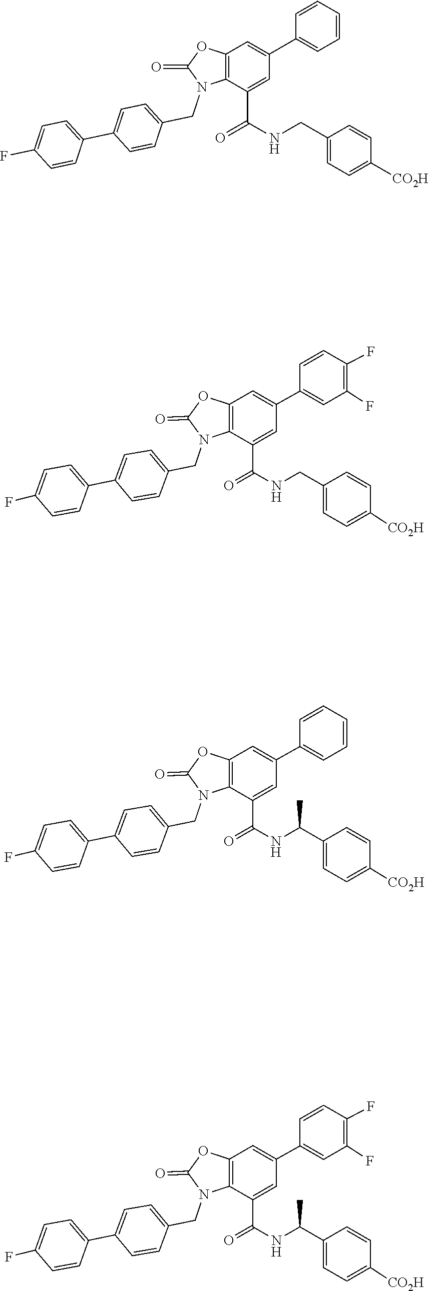 [SMILES: O=C(O)c1ccc(CNC(=O)c2cc(-c3ccccc3)cc3oc(=O)n(Cc4ccc(-c5ccc(F)cc5)cc4)c23)cc1 ; O=C(O)c1ccc(CNC(=O)c2cc(-c3ccc(F)c(F)c3)cc3oc(=O)n(Cc4ccc(-c5ccc(F)cc5)cc4)c23)cc1 ; C[C@H](NC(=O)c1cc(-c2ccccc2)cc2oc(=O)n(Cc3ccc(-c4ccc(F)cc4)cc3)c12)c1ccc(C(=O)O)cc1 ; C[C@H](NC(=O)c1cc(-c2ccc(F)c(F)c2)cc2oc(=O)n(Cc3ccc(-c4ccc(F)cc4)cc3)c12)c1ccc(C(=O)O)cc1]

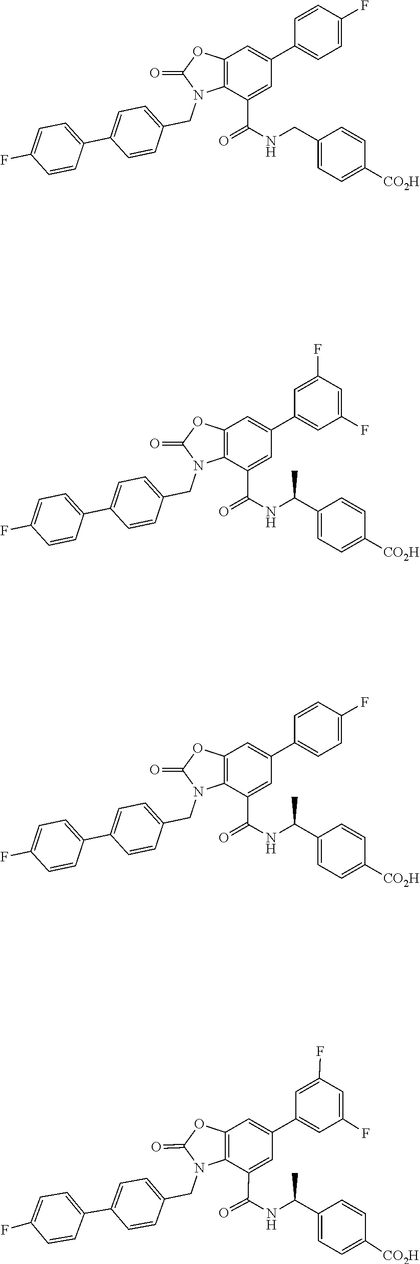 [SMILES: O=C(O)c1ccc(CNC(=O)c2cc(-c3ccc(F)cc3)cc3oc(=O)n(Cc4ccc(-c5ccc(F)cc5)cc4)c23)cc1 ; C[C@H](NC(=O)c1cc(-c2cc(F)cc(F)c2)cc2oc(=O)n(Cc3ccc(-c4ccc(F)cc4)cc3)c12)c1ccc(C(=O)O)cc1 ; C[C@H](NC(=O)c1cc(-c2ccc(F)cc2)cc2oc(=O)n(Cc3ccc(-c4ccc(F)cc4)cc3)c12)c1ccc(C(=O)O)cc1 ; C[C@H](NC(=O)c1cc(-c2cc(F)cc(F)c2)cc2oc(=O)n(Cc3ccc(-c4ccc(F)cc4)cc3)c12)c1ccc(C(=O)O)cc1]

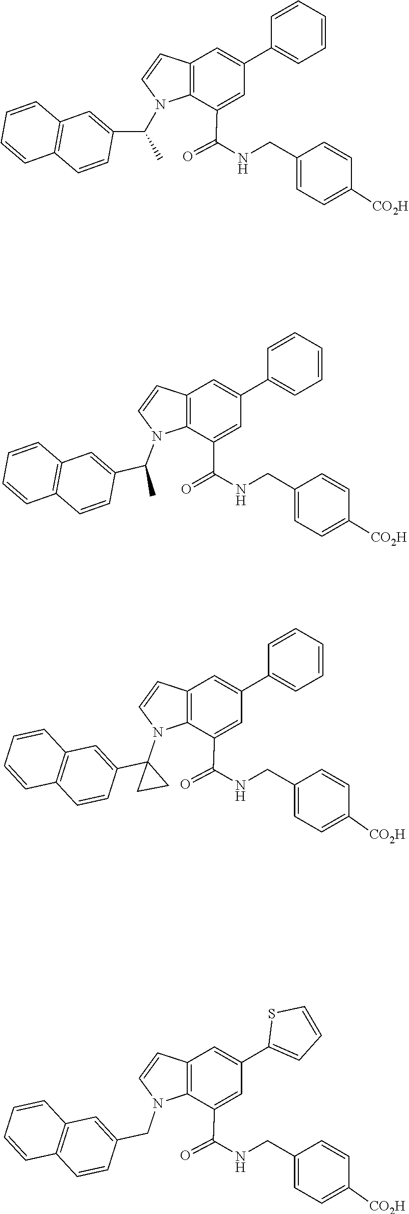 [SMILES: C[C@H](c1ccc2ccccc2c1)n1ccc2cc(-c3ccccc3)cc(C(=O)NCc3ccc(C(=O)O)cc3)c21 ; C[C@@H](c1ccc2ccccc2c1)n1ccc2cc(-c3ccccc3)cc(C(=O)NCc3ccc(C(=O)O)cc3)c21 ; O=C(O)c1ccc(CNC(=O)c2cc(-c3ccccc3)cc3ccn(C4(c5ccc6ccccc6c5)CC4)c23)cc1 ; O=C(O)c1ccc(CNC(=O)c2cc(-c3cccs3)cc3ccn(Cc4ccc5ccccc5c4)c23)cc1]

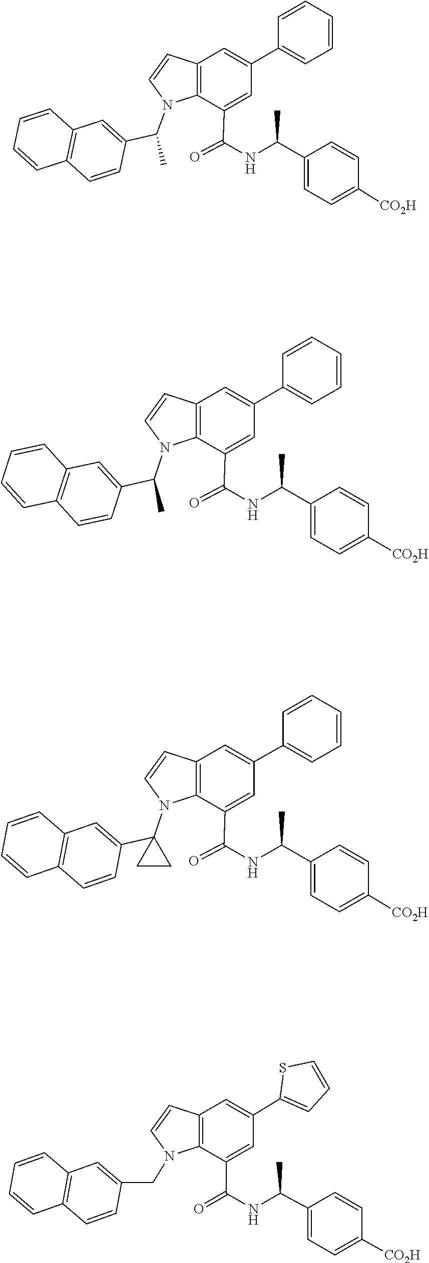 [SMILES: C[C@H](NC(=O)c1cc(-c2ccccc2)cc2ccn([C@H](C)c3ccc4ccccc4c3)c12)c1ccc(C(=O)O)cc1 ; C[C@H](NC(=O)c1cc(-c2ccccc2)cc2ccn([C@@H](C)c3ccc4ccccc4c3)c12)c1ccc(C(=O)O)cc1 ; C[C@H](NC(=O)c1cc(-c2ccccc2)cc2ccn(C3(c4ccc5ccccc5c4)CC3)c12)c1ccc(C(=O)O)cc1 ; C[C@H](NC(=O)c1cc(-c2cccs2)cc2ccn(Cc3ccc4ccccc4c3)c12)c1ccc(C(=O)O)cc1]

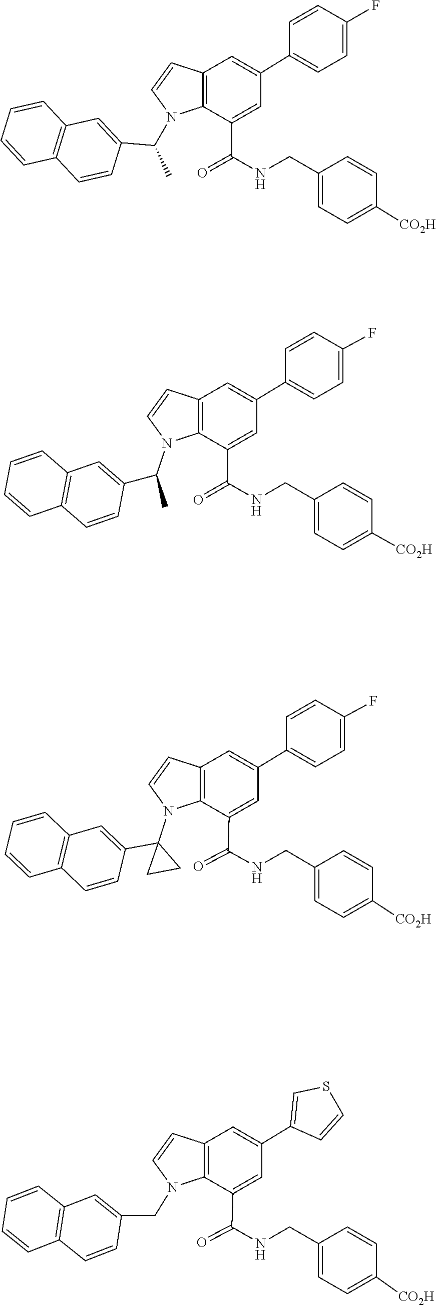 [SMILES: C[C@H](c1ccc2ccccc2c1)n1ccc2cc(-c3ccc(F)cc3)cc(C(=O)NCc3ccc(C(=O)O)cc3)c21 ; C[C@@H](c1ccc2ccccc2c1)n1ccc2cc(-c3ccc(F)cc3)cc(C(=O)NCc3ccc(C(=O)O)cc3)c21 ; O=C(O)c1ccc(CNC(=O)c2cc(-c3ccc(F)cc3)cc3ccn(C4(c5ccc6ccccc6c5)CC4)c23)cc1 ; O=C(O)c1ccc(CNC(=O)c2cc(-c3ccsc3)cc3ccn(Cc4ccc5ccccc5c4)c23)cc1]

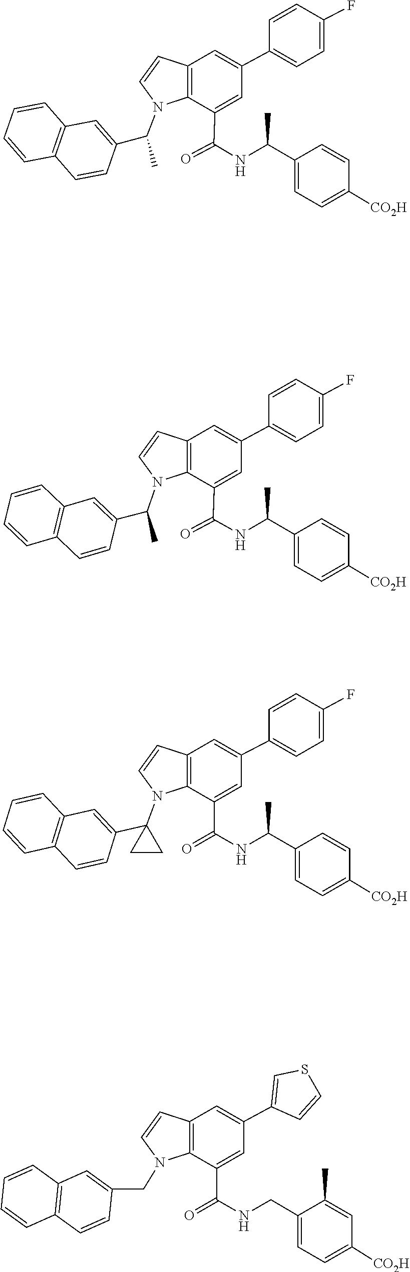

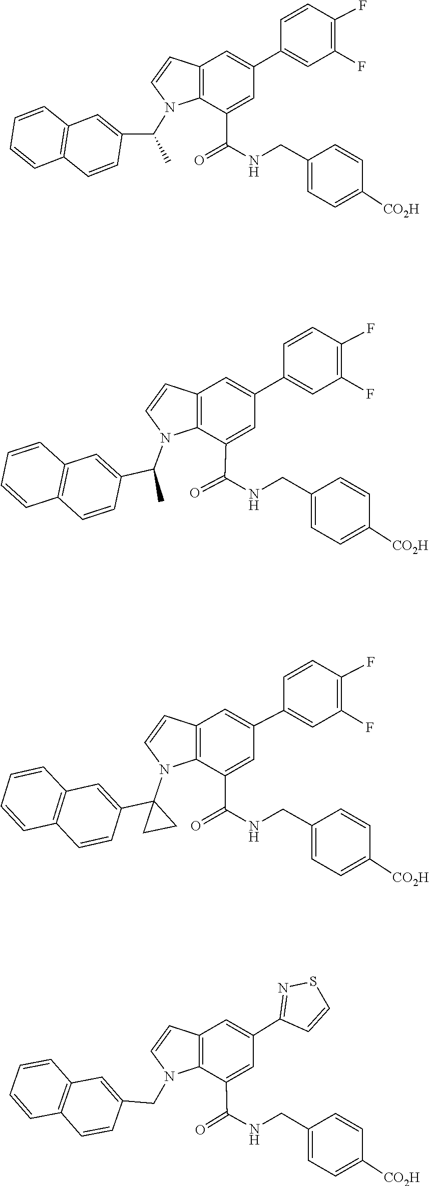 [SMILES: C[C@H](c1ccc2ccccc2c1)n1ccc2cc(-c3ccc(F)c(F)c3)cc(C(=O)NCc3ccc(C(=O)O)cc3)c21 ; C[C@@H](c1ccc2ccccc2c1)n1ccc2cc(-c3ccc(F)c(F)c3)cc(C(=O)NCc3ccc(C(=O)O)cc3)c21 ; O=C(O)c1ccc(CNC(=O)c2cc(-c3ccc(F)c(F)c3)cc3ccn(C4(c5ccc6ccccc6c5)CC4)c23)cc1 ; O=C(O)c1ccc(CNC(=O)c2cc(-c3ccsn3)cc3ccn(Cc4ccc5ccccc5c4)c23)cc1]

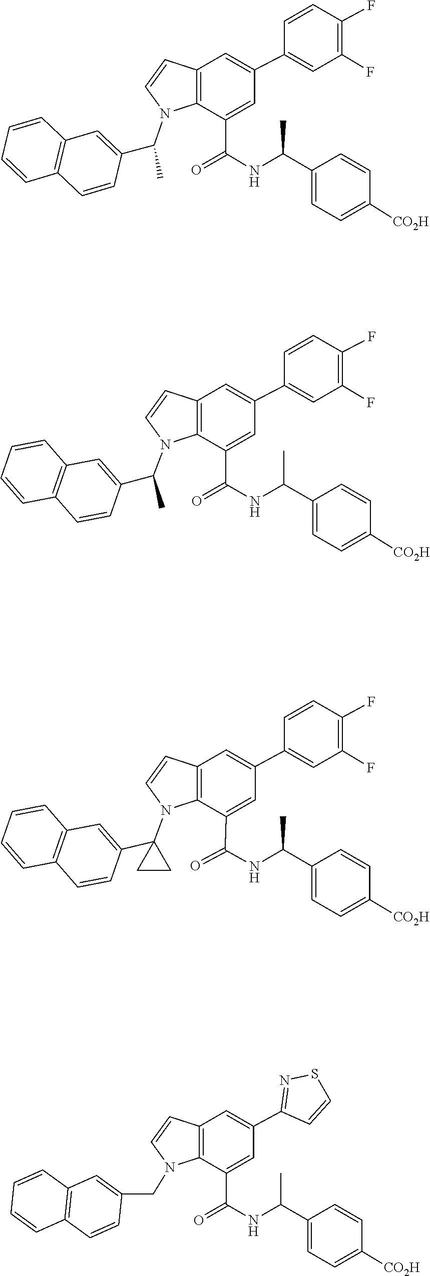 [SMILES: C[C@H](NC(=O)c1cc(-c2ccc(F)c(F)c2)cc2ccn([C@H](C)c3ccc4ccccc4c3)c12)c1ccc(C(=O)O)cc1 ; CC(NC(=O)c1cc(-c2ccc(F)c(F)c2)cc2ccn([C@@H](C)c3ccc4ccccc4c3)c12)c1ccc(C(=O)O)cc1 ; C[C@H](NC(=O)c1cc(-c2ccc(F)c(F)c2)cc2ccn(C3(c4ccc5ccccc5c4)CC3)c12)c1ccc(C(=O)O)cc1 ; CC(NC(=O)c1cc(-c2ccsn2)cc2ccn(Cc3ccc4ccccc4c3)c12)c1ccc(C(=O)O)cc1]

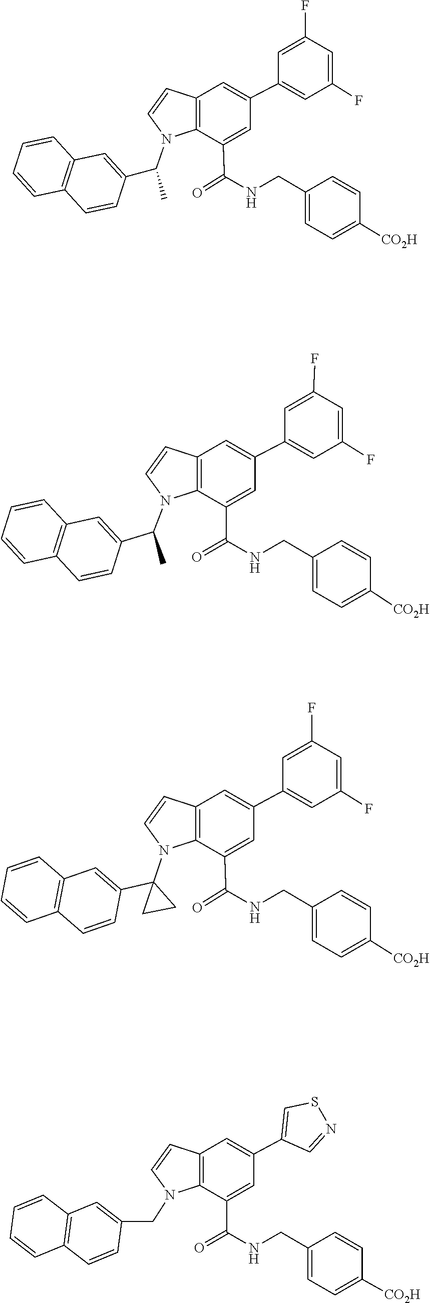 [SMILES: C[C@H](c1ccc2ccccc2c1)n1ccc2cc(-c3cc(F)cc(F)c3)cc(C(=O)NCc3ccc(C(=O)O)cc3)c21 ; C[C@@H](c1ccc2ccccc2c1)n1ccc2cc(-c3cc(F)cc(F)c3)cc(C(=O)NCc3ccc(C(=O)O)cc3)c21 ; O=C(O)c1ccc(CNC(=O)c2cc(-c3cc(F)cc(F)c3)cc3ccn(C4(c5ccc6ccccc6c5)CC4)c23)cc1 ; O=C(O)c1ccc(CNC(=O)c2cc(-c3cnsc3)cc3ccn(Cc4ccc5ccccc5c4)c23)cc1]

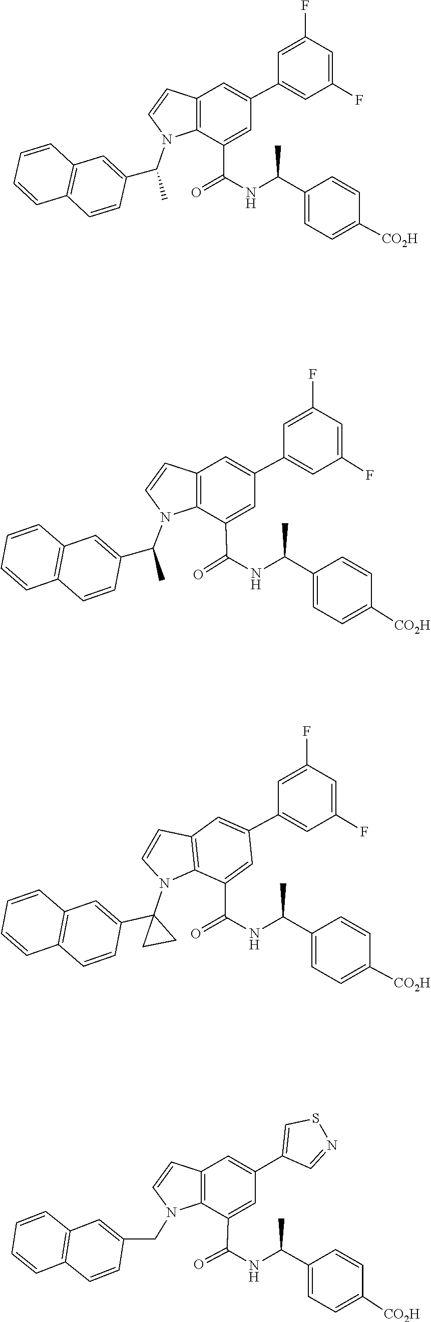 [SMILES: C[C@H](NC(=O)c1cc(-c2cc(F)cc(F)c2)cc2ccn([C@H](C)c3ccc4ccccc4c3)c12)c1ccc(C(=O)O)cc1 ; C[C@H](NC(=O)c1cc(-c2cc(F)cc(F)c2)cc2ccn([C@@H](C)c3ccc4ccccc4c3)c12)c1ccc(C(=O)O)cc1 ; C[C@H](NC(=O)c1cc(-c2cc(F)cc(F)c2)cc2ccn(C3(c4ccc5ccccc5c4)CC3)c12)c1ccc(C(=O)O)cc1 ; C[C@H](NC(=O)c1cc(-c2cnsc2)cc2ccn(Cc3ccc4ccccc4c3)c12)c1ccc(C(=O)O)cc1]

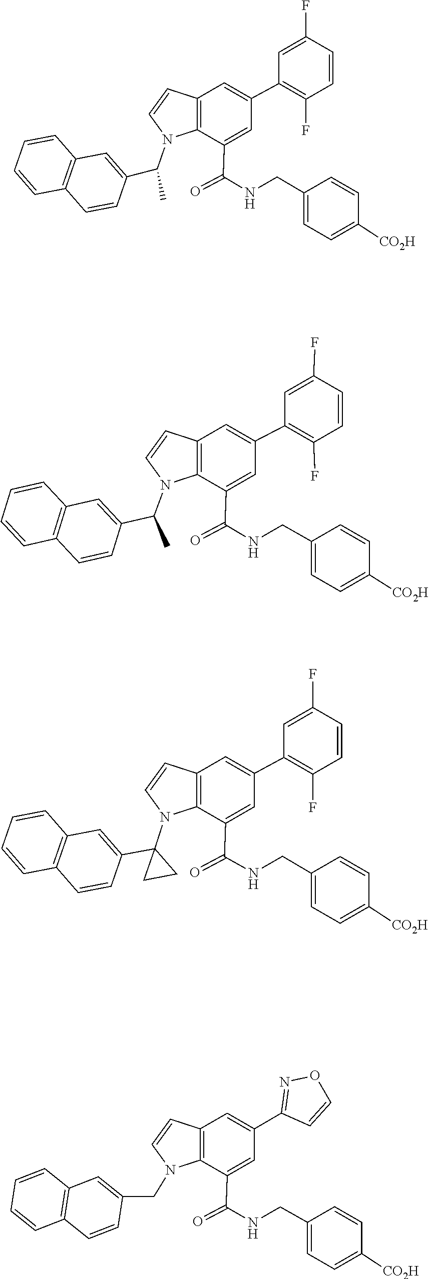 [SMILES: C[C@H](c1ccc2ccccc2c1)n1ccc2cc(-c3cc(F)ccc3F)cc(C(=O)NCc3ccc(C(=O)O)cc3)c21 ; C[C@@H](c1ccc2ccccc2c1)n1ccc2cc(-c3cc(F)ccc3F)cc(C(=O)NCc3ccc(C(=O)O)cc3)c21 ; O=C(O)c1ccc(CNC(=O)c2cc(-c3cc(F)ccc3F)cc3ccn(C4(c5ccc6ccccc6c5)CC4)c23)cc1 ; O=C(O)c1ccc(CNC(=O)c2cc(-c3ccon3)cc3ccn(Cc4ccc5ccccc5c4)c23)cc1]

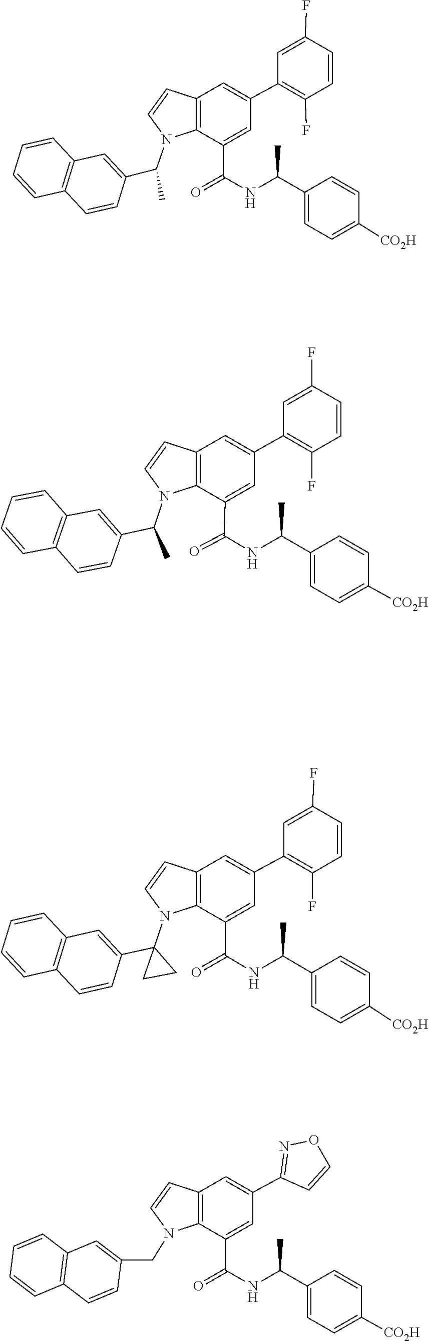 [SMILES: C[C@H](NC(=O)c1cc(-c2cc(F)ccc2F)cc2ccn([C@H](C)c3ccc4ccccc4c3)c12)c1ccc(C(=O)O)cc1 ; C[C@H](NC(=O)c1cc(-c2cc(F)ccc2F)cc2ccn([C@@H](C)c3ccc4ccccc4c3)c12)c1ccc(C(=O)O)cc1 ; C[C@H](NC(=O)c1cc(-c2cc(F)ccc2F)cc2ccn(C3(c4ccc5ccccc5c4)CC3)c12)c1ccc(C(=O)O)cc1 ; C[C@H](NC(=O)c1cc(-c2ccon2)cc2ccn(Cc3ccc4ccccc4c3)c12)c1ccc(C(=O)O)cc1]

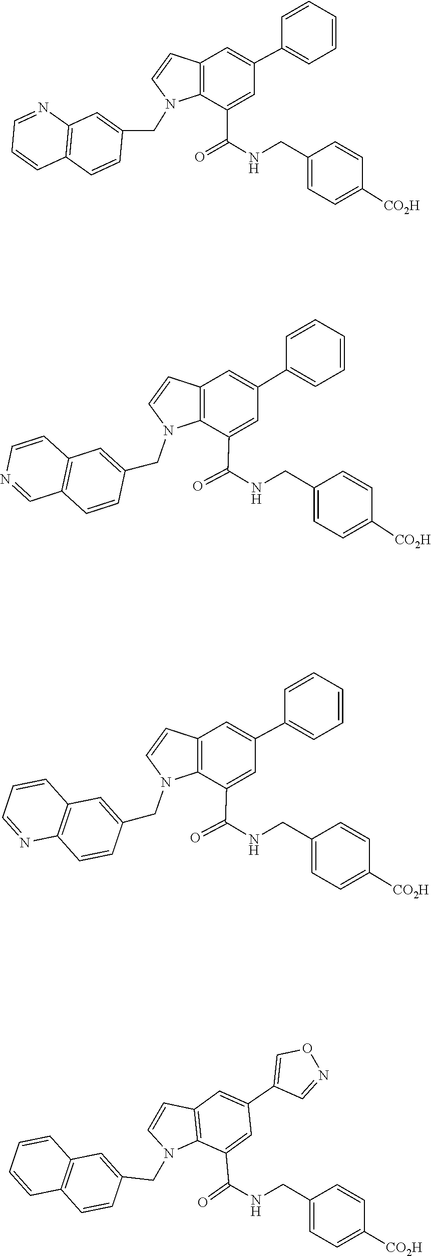 [SMILES: O=C(O)c1ccc(CNC(=O)c2cc(-c3ccccc3)cc3ccn(Cc4ccc5cccnc5c4)c23)cc1 ; O=C(O)c1ccc(CNC(=O)c2cc(-c3ccccc3)cc3ccn(Cc4ccc5cnccc5c4)c23)cc1 ; O=C(O)c1ccc(CNC(=O)c2cc(-c3ccccc3)cc3ccn(Cc4ccc5ncccc5c4)c23)cc1 ; O=C(O)c1ccc(CNC(=O)c2cc(-c3cnoc3)cc3ccn(Cc4ccc5ccccc5c4)c23)cc1]

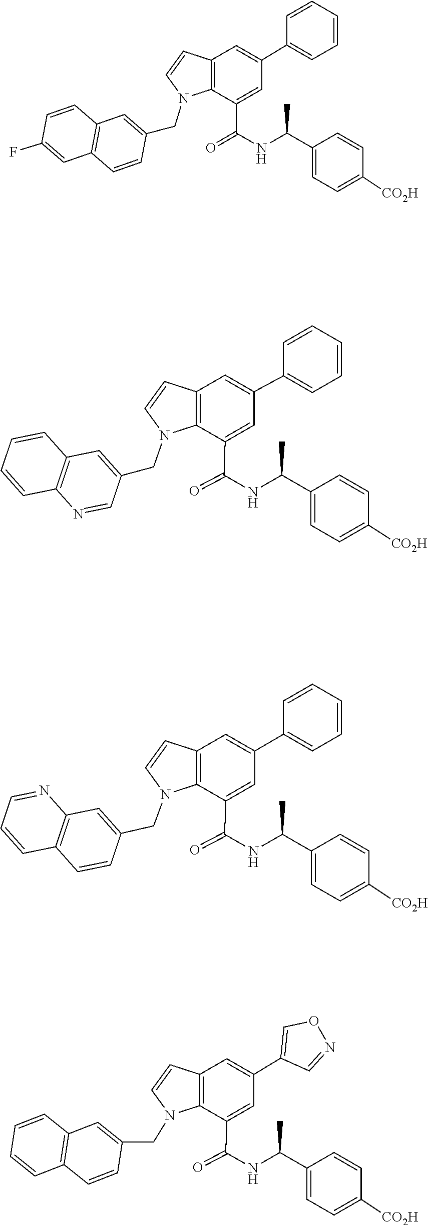 [SMILES: C[C@H](NC(=O)c1cc(-c2ccccc2)cc2ccn(Cc3ccc4cc(F)ccc4c3)c12)c1ccc(C(=O)O)cc1 ; C[C@H](NC(=O)c1cc(-c2ccccc2)cc2ccn(Cc3cnc4ccccc4c3)c12)c1ccc(C(=O)O)cc1 ; C[C@H](NC(=O)c1cc(-c2ccccc2)cc2ccn(Cc3ccc4cccnc4c3)c12)c1ccc(C(=O)O)cc1 ; C[C@H](NC(=O)c1cc(-c2cnoc2)cc2ccn(Cc3ccc4ccccc4c3)c12)c1ccc(C(=O)O)cc1]

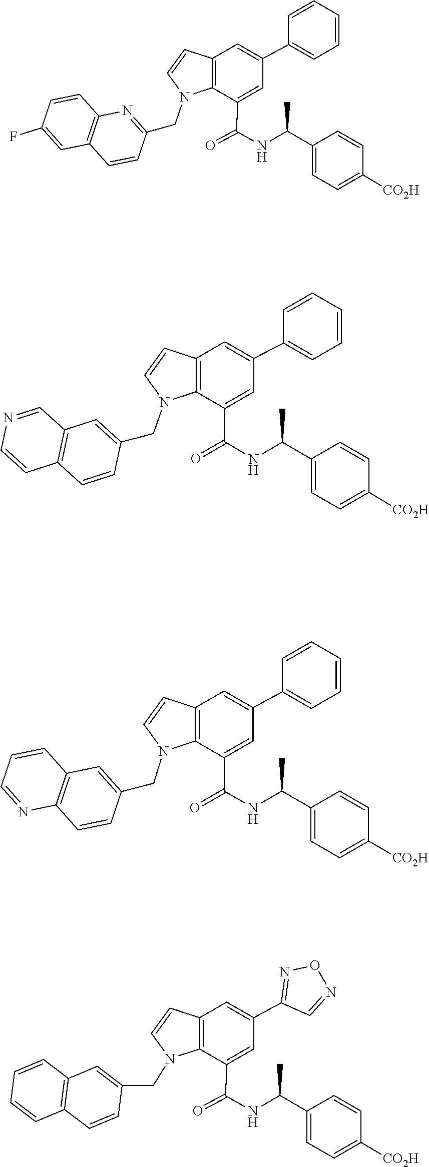 [SMILES: C[C@H](NC(=O)c1cc(-c2ccccc2)cc2ccn(Cc3ccc4cc(F)ccc4n3)c12)c1ccc(C(=O)O)cc1 ; C[C@H](NC(=O)c1cc(-c2ccccc2)cc2ccn(Cc3ccc4ccncc4c3)c12)c1ccc(C(=O)O)cc1 ; C[C@H](NC(=O)c1cc(-c2ccccc2)cc2ccn(Cc3ccc4ncccc4c3)c12)c1ccc(C(=O)O)cc1 ; C[C@H](NC(=O)c1cc(-c2cnon2)cc2ccn(Cc3ccc4ccccc4c3)c12)c1ccc(C(=O)O)cc1]

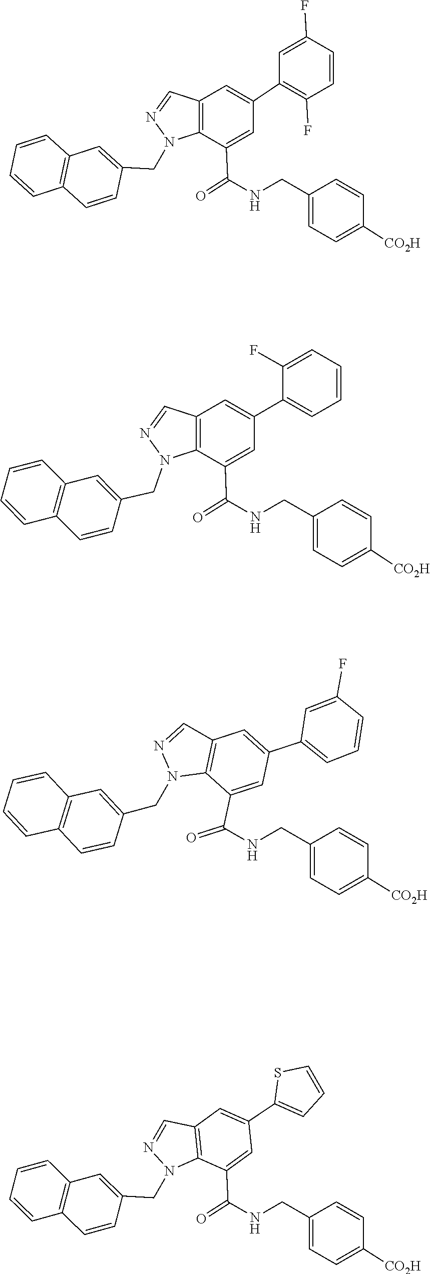 [SMILES: O=C(O)c1ccc(CNC(=O)c2cc(-c3cc(F)ccc3F)cc3cnn(Cc4ccc5ccccc5c4)c23)cc1 ; O=C(O)c1ccc(CNC(=O)c2cc(-c3ccccc3F)cc3cnn(Cc4ccc5ccccc5c4)c23)cc1 ; O=C(O)c1ccc(CNC(=O)c2cc(-c3cccc(F)c3)cc3cnn(Cc4ccc5ccccc5c4)c23)cc1 ; O=C(O)c1ccc(CNC(=O)c2cc(-c3cccs3)cc3cnn(Cc4ccc5ccccc5c4)c23)cc1]

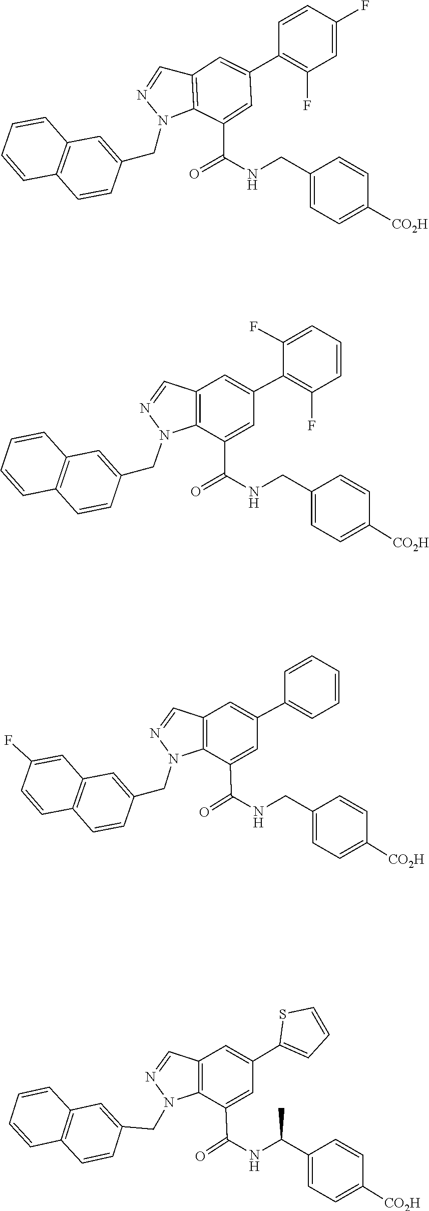 [SMILES: O=C(O)c1ccc(CNC(=O)c2cc(-c3ccc(F)cc3F)cc3cnn(Cc4ccc5ccccc5c4)c23)cc1 ; O=C(O)c1ccc(CNC(=O)c2cc(-c3c(F)cccc3F)cc3cnn(Cc4ccc5ccccc5c4)c23)cc1 ; O=C(O)c1ccc(CNC(=O)c2cc(-c3ccccc3)cc3cnn(Cc4ccc5ccc(F)cc5c4)c23)cc1 ; C[C@H](NC(=O)c1cc(-c2cccs2)cc2cnn(Cc3ccc4ccccc4c3)c12)c1ccc(C(=O)O)cc1]

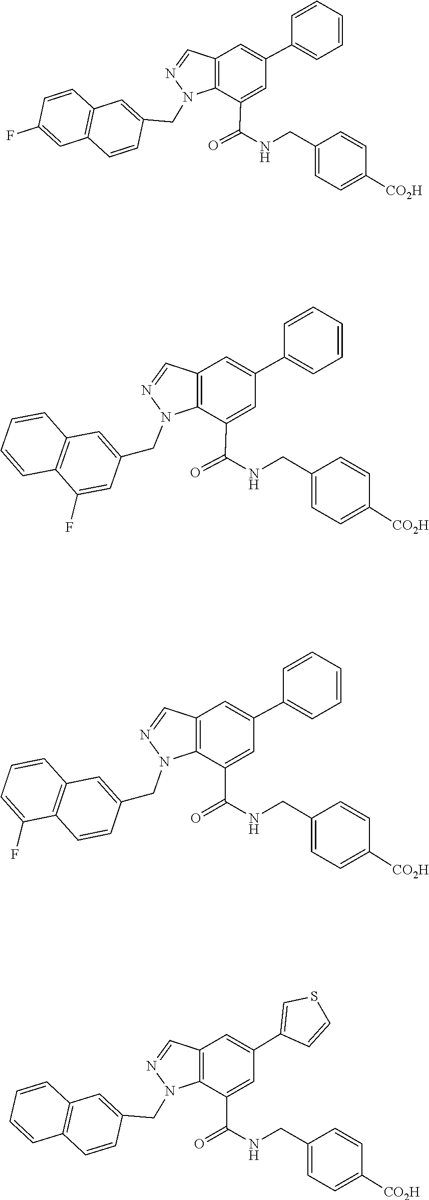 [SMILES: O=C(O)c1ccc(CNC(=O)c2cc(-c3ccccc3)cc3cnn(Cc4ccc5cc(F)ccc5c4)c23)cc1 ; O=C(O)c1ccc(CNC(=O)c2cc(-c3ccccc3)cc3cnn(Cc4cc(F)c5ccccc5c4)c23)cc1 ; O=C(O)c1ccc(CNC(=O)c2cc(-c3ccccc3)cc3cnn(Cc4ccc5c(F)cccc5c4)c23)cc1 ; O=C(O)c1ccc(CNC(=O)c2cc(-c3ccsc3)cc3cnn(Cc4ccc5ccccc5c4)c23)cc1]

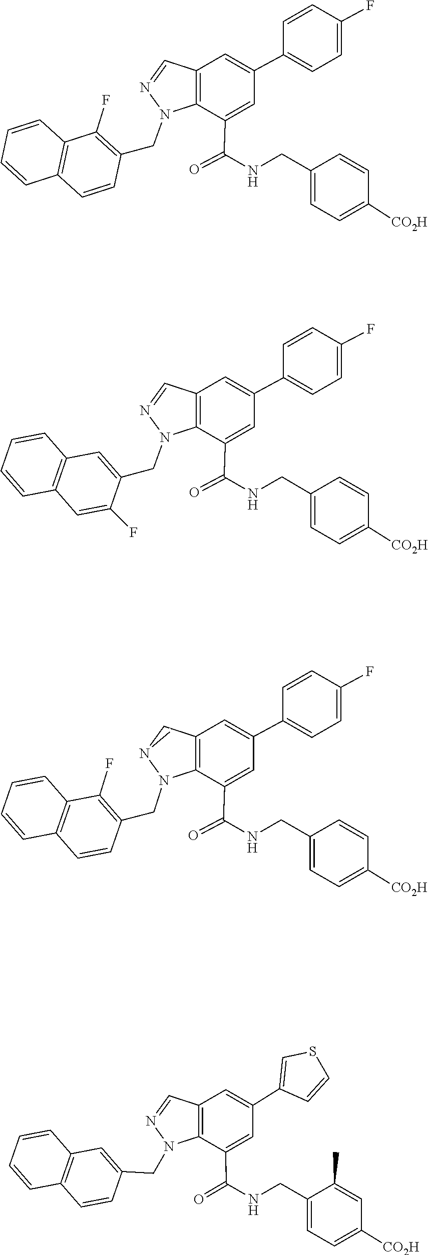

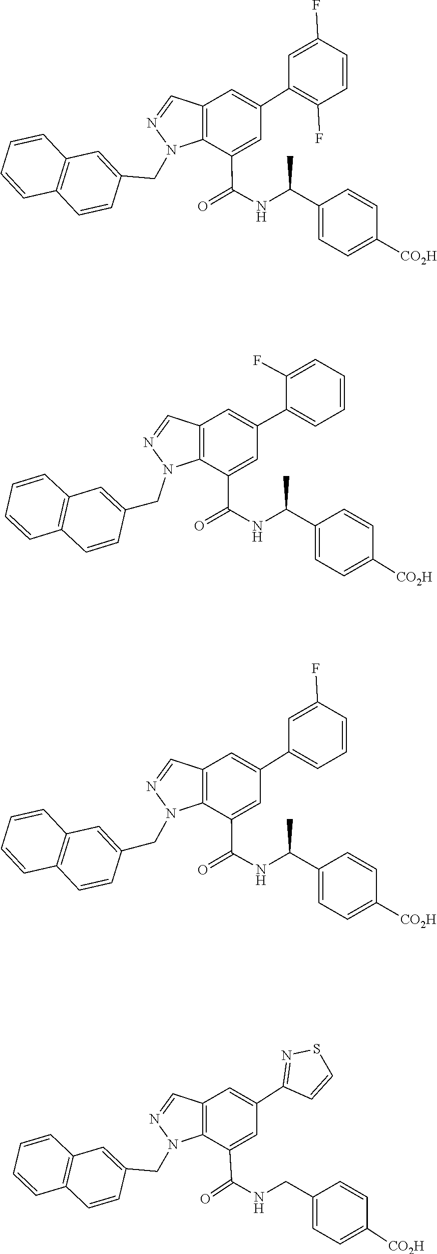 [SMILES: C[C@H](NC(=O)c1cc(-c2cc(F)ccc2F)cc2cnn(Cc3ccc4ccccc4c3)c12)c1ccc(C(=O)O)cc1 ; C[C@H](NC(=O)c1cc(-c2ccccc2F)cc2cnn(Cc3ccc4ccccc4c3)c12)c1ccc(C(=O)O)cc1 ; C[C@H](NC(=O)c1cc(-c2cccc(F)c2)cc2cnn(Cc3ccc4ccccc4c3)c12)c1ccc(C(=O)O)cc1 ; O=C(O)c1ccc(CNC(=O)c2cc(-c3ccsn3)cc3cnn(Cc4ccc5ccccc5c4)c23)cc1]

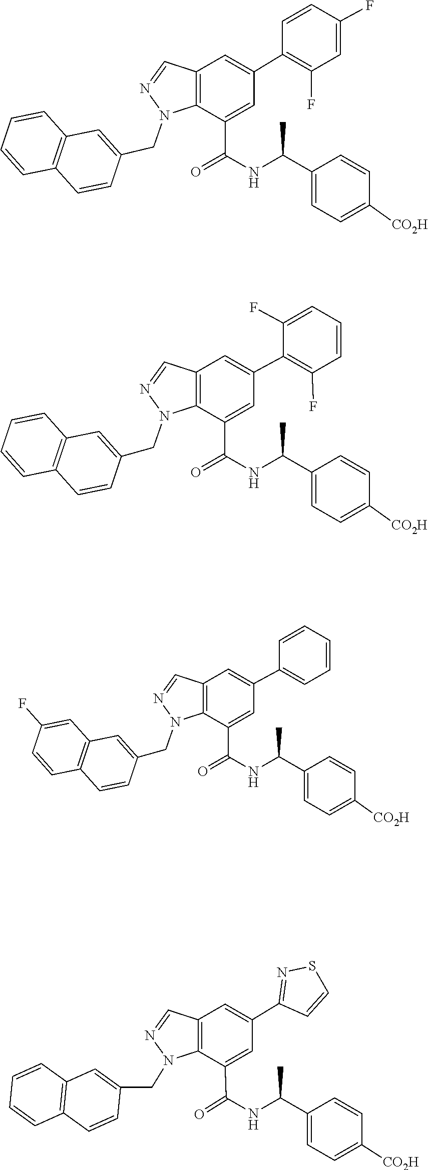 [SMILES: C[C@H](NC(=O)c1cc(-c2ccc(F)cc2F)cc2cnn(Cc3ccc4ccccc4c3)c12)c1ccc(C(=O)O)cc1 ; C[C@H](NC(=O)c1cc(-c2c(F)cccc2F)cc2cnn(Cc3ccc4ccccc4c3)c12)c1ccc(C(=O)O)cc1 ; C[C@H](NC(=O)c1cc(-c2ccccc2)cc2cnn(Cc3ccc4ccc(F)cc4c3)c12)c1ccc(C(=O)O)cc1 ; C[C@H](NC(=O)c1cc(-c2ccsn2)cc2cnn(Cc3ccc4ccccc4c3)c12)c1ccc(C(=O)O)cc1]

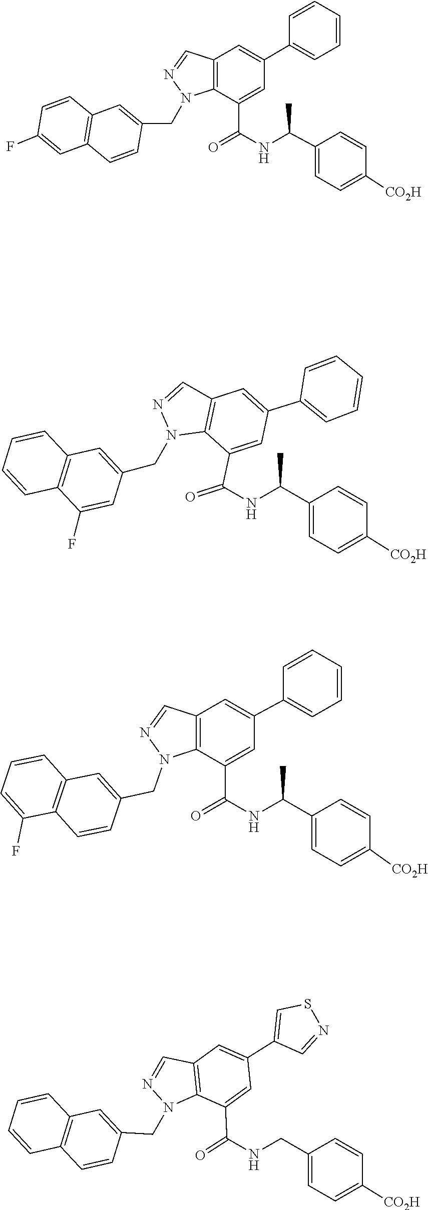 [SMILES: C[C@H](NC(=O)c1cc(-c2ccccc2)cc2cnn(Cc3ccc4cc(F)ccc4c3)c12)c1ccc(C(=O)O)cc1 ; C[C@H](NC(=O)c1cc(-c2ccccc2)cc2cnn(Cc3cc(F)c4ccccc4c3)c12)c1ccc(C(=O)O)cc1 ; C[C@H](NC(=O)c1cc(-c2ccccc2)cc2cnn(Cc3ccc4c(F)cccc4c3)c12)c1ccc(C(=O)O)cc1 ; O=C(O)c1ccc(CNC(=O)c2cc(-c3cnsc3)cc3cnn(Cc4ccc5ccccc5c4)c23)cc1]

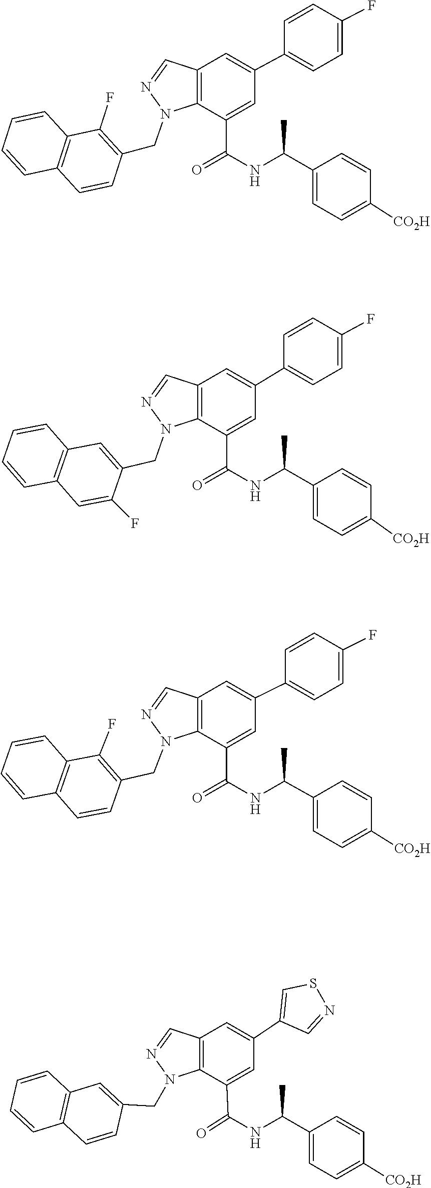 [SMILES: C[C@H](NC(=O)c1cc(-c2ccc(F)cc2)cc2cnn(Cc3ccc4ccccc4c3F)c12)c1ccc(C(=O)O)cc1 ; C[C@H](NC(=O)c1cc(-c2ccc(F)cc2)cc2cnn(Cc3cc4ccccc4cc3F)c12)c1ccc(C(=O)O)cc1 ; C[C@H](NC(=O)c1cc(-c2ccc(F)cc2)cc2cnn(Cc3ccc4ccccc4c3F)c12)c1ccc(C(=O)O)cc1 ; C[C@H](NC(=O)c1cc(-c2cnsc2)cc2cnn(Cc3ccc4ccccc4c3)c12)c1ccc(C(=O)O)cc1]

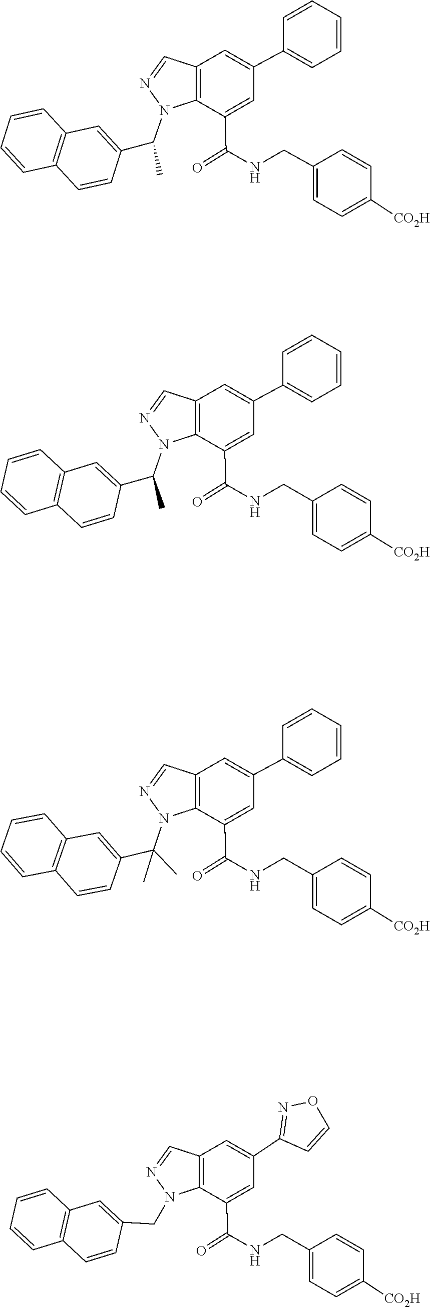 [SMILES: C[C@H](c1ccc2ccccc2c1)n1ncc2cc(-c3ccccc3)cc(C(=O)NCc3ccc(C(=O)O)cc3)c21 ; C[C@@H](c1ccc2ccccc2c1)n1ncc2cc(-c3ccccc3)cc(C(=O)NCc3ccc(C(=O)O)cc3)c21 ; CC(C)(c1ccc2ccccc2c1)n1ncc2cc(-c3ccccc3)cc(C(=O)NCc3ccc(C(=O)O)cc3)c21 ; O=C(O)c1ccc(CNC(=O)c2cc(-c3ccon3)cc3cnn(Cc4ccc5ccccc5c4)c23)cc1]

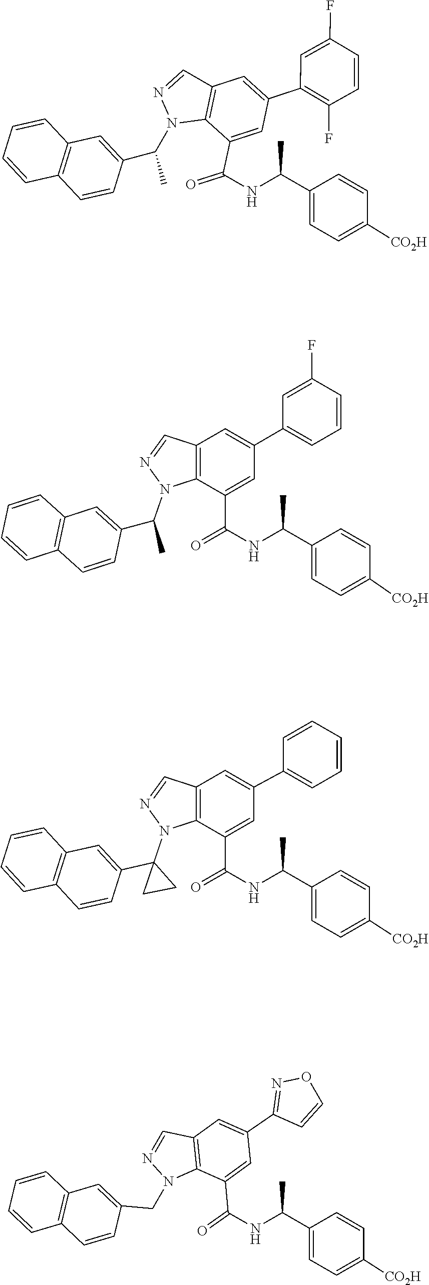 [SMILES: C[C@H](NC(=O)c1cc(-c2cc(F)ccc2F)cc2cnn([C@H](C)c3ccc4ccccc4c3)c12)c1ccc(C(=O)O)cc1 ; C[C@H](NC(=O)c1cc(-c2cccc(F)c2)cc2cnn([C@@H](C)c3ccc4ccccc4c3)c12)c1ccc(C(=O)O)cc1 ; C[C@H](NC(=O)c1cc(-c2ccccc2)cc2cnn(C3(c4ccc5ccccc5c4)CC3)c12)c1ccc(C(=O)O)cc1 ; C[C@H](NC(=O)c1cc(-c2ccon2)cc2cnn(Cc3ccc4ccccc4c3)c12)c1ccc(C(=O)O)cc1]

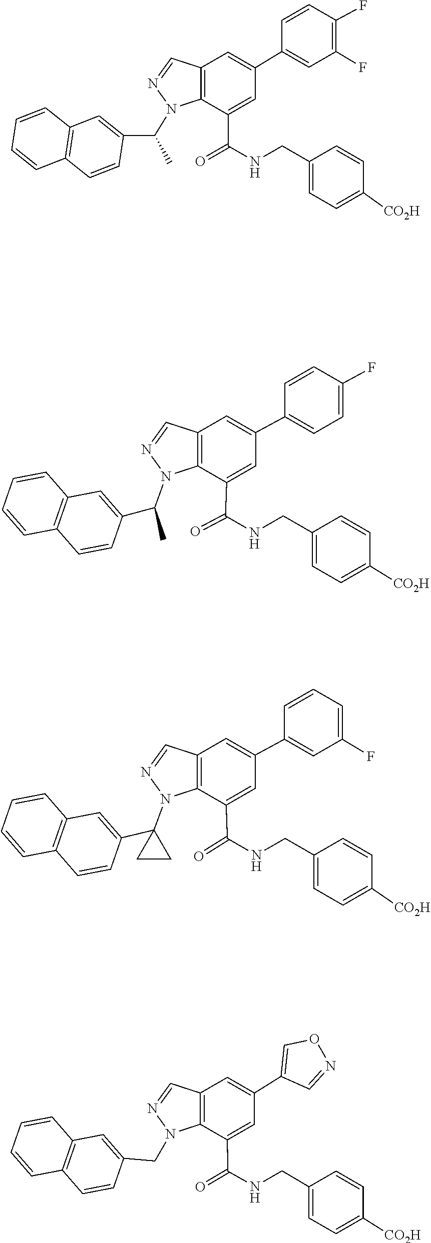 [SMILES: C[C@H](c1ccc2ccccc2c1)n1ncc2cc(-c3ccc(F)c(F)c3)cc(C(=O)NCc3ccc(C(=O)O)cc3)c21 ; C[C@@H](c1ccc2ccccc2c1)n1ncc2cc(-c3ccc(F)cc3)cc(C(=O)NCc3ccc(C(=O)O)cc3)c21 ; O=C(O)c1ccc(CNC(=O)c2cc(-c3cccc(F)c3)cc3cnn(C4(c5ccc6ccccc6c5)CC4)c23)cc1 ; O=C(O)c1ccc(CNC(=O)c2cc(-c3cnoc3)cc3cnn(Cc4ccc5ccccc5c4)c23)cc1]

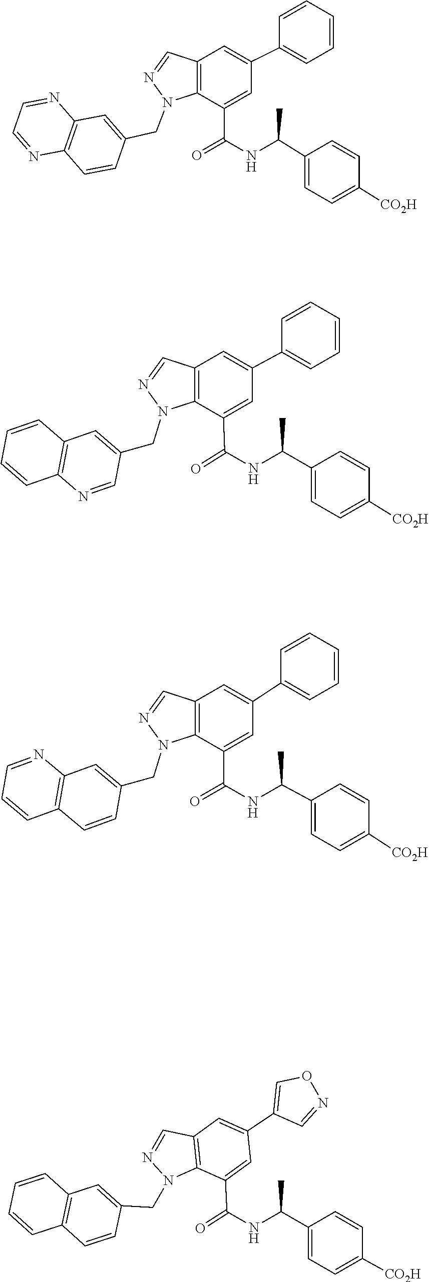 [SMILES: C[C@H](NC(=O)c1cc(-c2ccccc2)cc2cnn(Cc3ccc4nccnc4c3)c12)c1ccc(C(=O)O)cc1 ; C[C@H](NC(=O)c1cc(-c2ccccc2)cc2cnn(Cc3cnc4ccccc4c3)c12)c1ccc(C(=O)O)cc1 ; C[C@H](NC(=O)c1cc(-c2ccccc2)cc2cnn(Cc3ccc4cccnc4c3)c12)c1ccc(C(=O)O)cc1 ; C[C@H](NC(=O)c1cc(-c2cnoc2)cc2cnn(Cc3ccc4ccccc4c3)c12)c1ccc(C(=O)O)cc1]

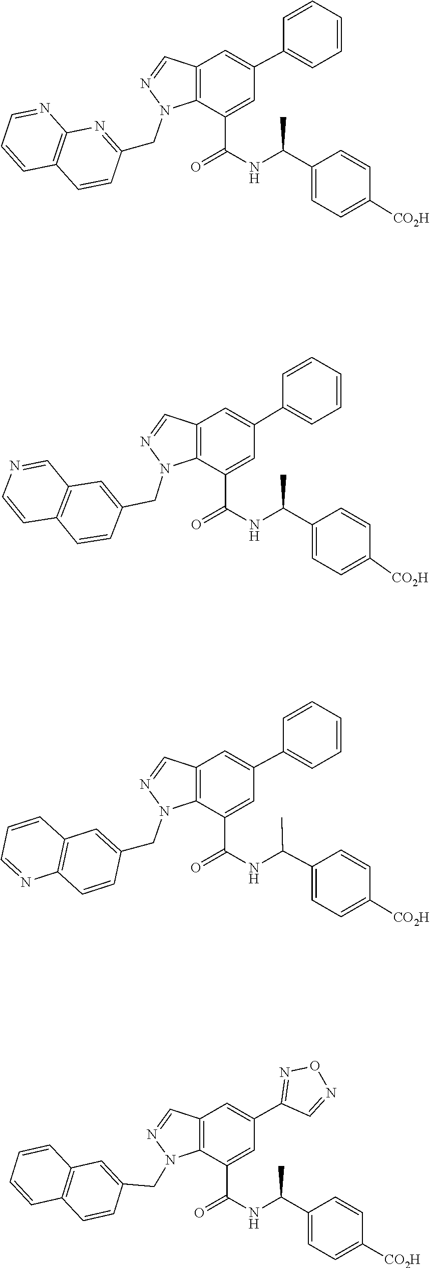 [SMILES: C[C@H](NC(=O)c1cc(-c2ccccc2)cc2cnn(Cc3ccc4cccnc4n3)c12)c1ccc(C(=O)O)cc1 ; C[C@H](NC(=O)c1cc(-c2ccccc2)cc2cnn(Cc3ccc4ccncc4c3)c12)c1ccc(C(=O)O)cc1 ; CC(NC(=O)c1cc(-c2ccccc2)cc2cnn(Cc3ccc4ncccc4c3)c12)c1ccc(C(=O)O)cc1 ; C[C@H](NC(=O)c1cc(-c2cnon2)cc2cnn(Cc3ccc4ccccc4c3)c12)c1ccc(C(=O)O)cc1]

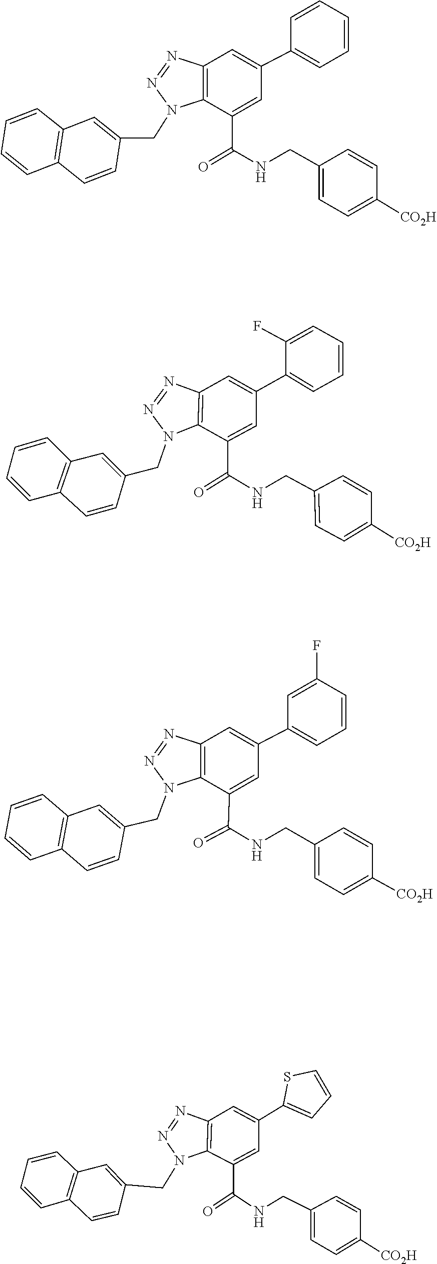 [SMILES: O=C(O)c1ccc(CNC(=O)c2cc(-c3ccccc3)cc3nnn(Cc4ccc5ccccc5c4)c23)cc1 ; O=C(O)c1ccc(CNC(=O)c2cc(-c3ccccc3F)cc3nnn(Cc4ccc5ccccc5c4)c23)cc1 ; O=C(O)c1ccc(CNC(=O)c2cc(-c3cccc(F)c3)cc3nnn(Cc4ccc5ccccc5c4)c23)cc1 ; O=C(O)c1ccc(CNC(=O)c2cc(-c3cccs3)cc3nnn(Cc4ccc5ccccc5c4)c23)cc1]

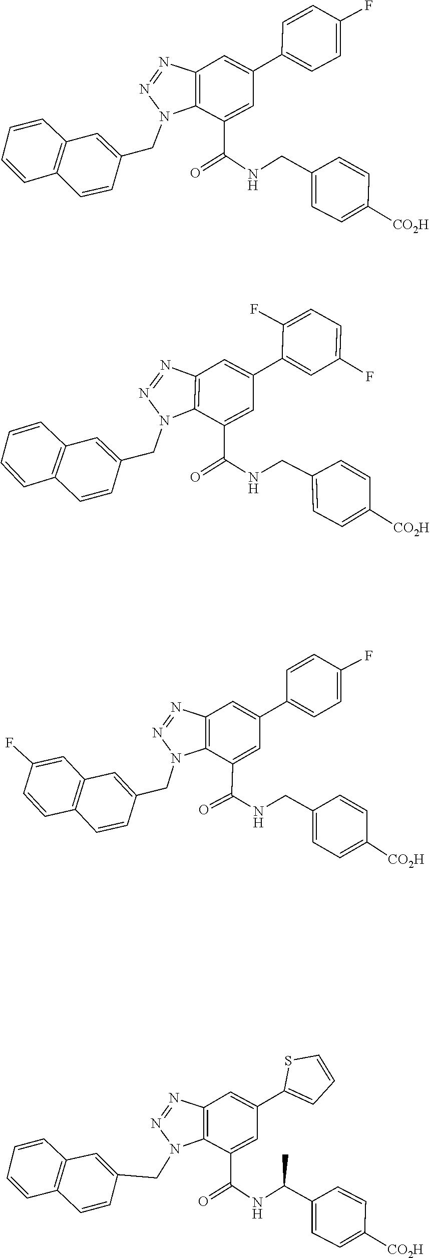 [SMILES: O=C(O)c1ccc(CNC(=O)c2cc(-c3ccc(F)cc3)cc3nnn(Cc4ccc5ccccc5c4)c23)cc1 ; O=C(O)c1ccc(CNC(=O)c2cc(-c3cc(F)ccc3F)cc3nnn(Cc4ccc5ccccc5c4)c23)cc1 ; O=C(O)c1ccc(CNC(=O)c2cc(-c3ccc(F)cc3)cc3nnn(Cc4ccc5ccc(F)cc5c4)c23)cc1 ; C[C@H](NC(=O)c1cc(-c2cccs2)cc2nnn(Cc3ccc4ccccc4c3)c12)c1ccc(C(=O)O)cc1]

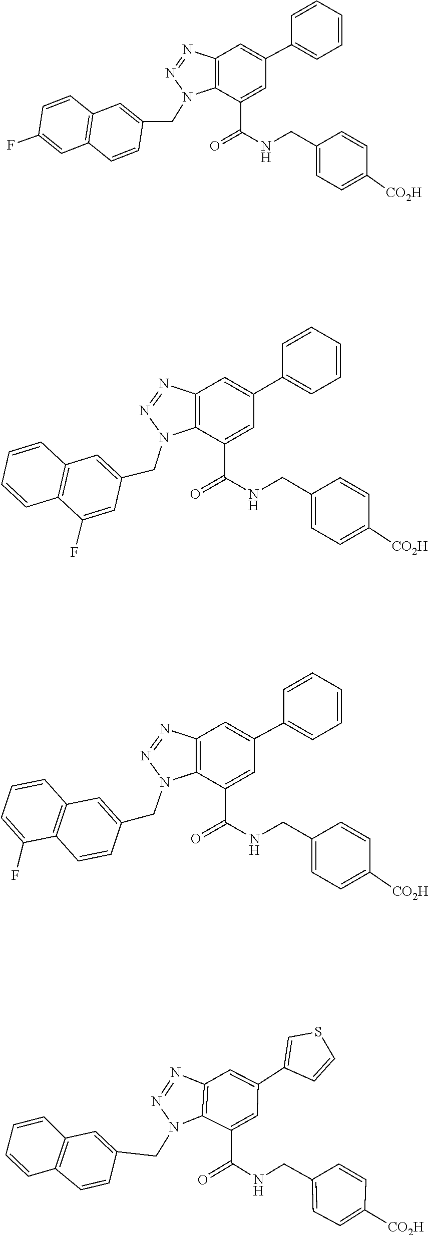 [SMILES: O=C(O)c1ccc(CNC(=O)c2cc(-c3ccccc3)cc3nnn(Cc4ccc5cc(F)ccc5c4)c23)cc1 ; O=C(O)c1ccc(CNC(=O)c2cc(-c3ccccc3)cc3nnn(Cc4cc(F)c5ccccc5c4)c23)cc1 ; O=C(O)c1ccc(CNC(=O)c2cc(-c3ccccc3)cc3nnn(Cc4ccc5c(F)cccc5c4)c23)cc1 ; O=C(O)c1ccc(CNC(=O)c2cc(-c3ccsc3)cc3nnn(Cc4ccc5ccccc5c4)c23)cc1]

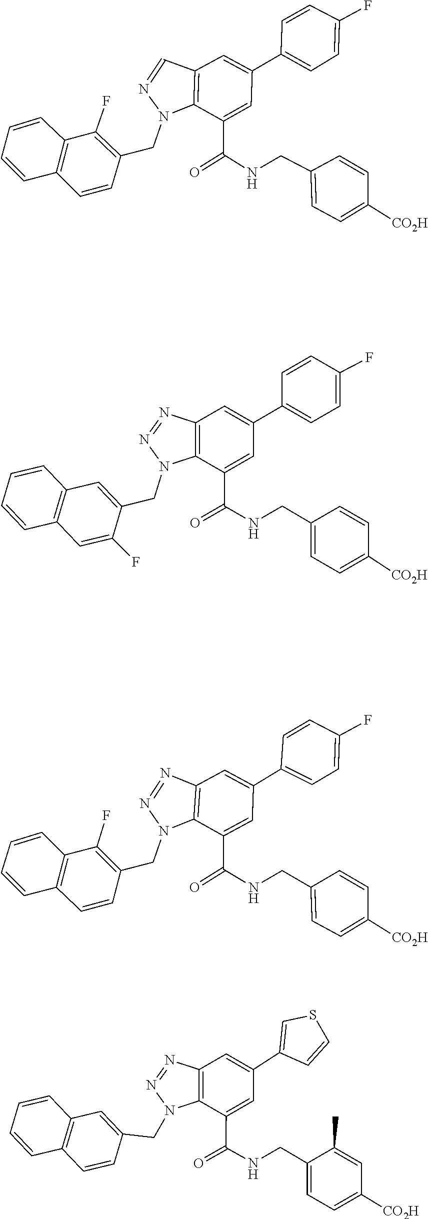

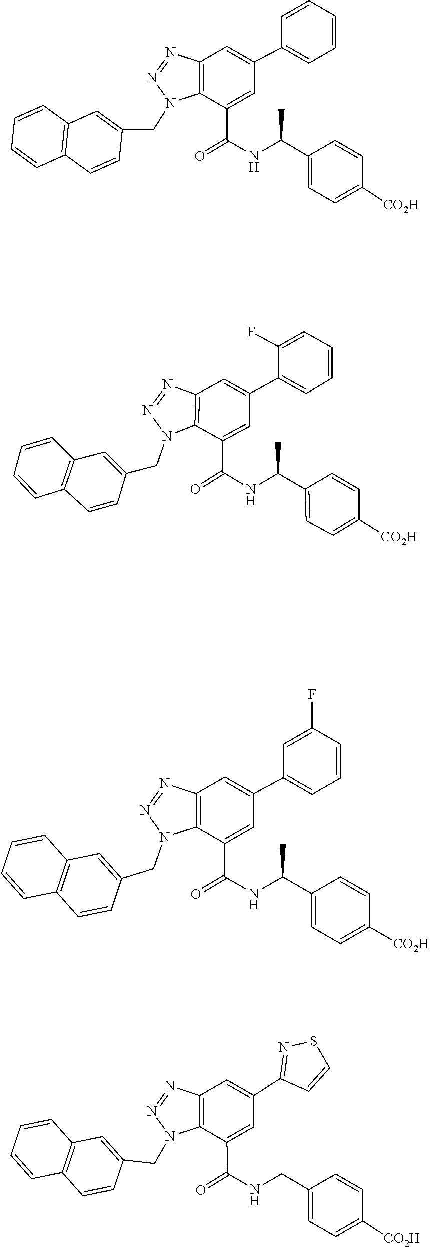 [SMILES: C[C@H](NC(=O)c1cc(-c2ccccc2)cc2nnn(Cc3ccc4ccccc4c3)c12)c1ccc(C(=O)O)cc1 ; C[C@H](NC(=O)c1cc(-c2ccccc2F)cc2nnn(Cc3ccc4ccccc4c3)c12)c1ccc(C(=O)O)cc1 ; C[C@H](NC(=O)c1cc(-c2cccc(F)c2)cc2nnn(Cc3ccc4ccccc4c3)c12)c1ccc(C(=O)O)cc1 ; O=C(O)c1ccc(CNC(=O)c2cc(-c3ccsn3)cc3nnn(Cc4ccc5ccccc5c4)c23)cc1]

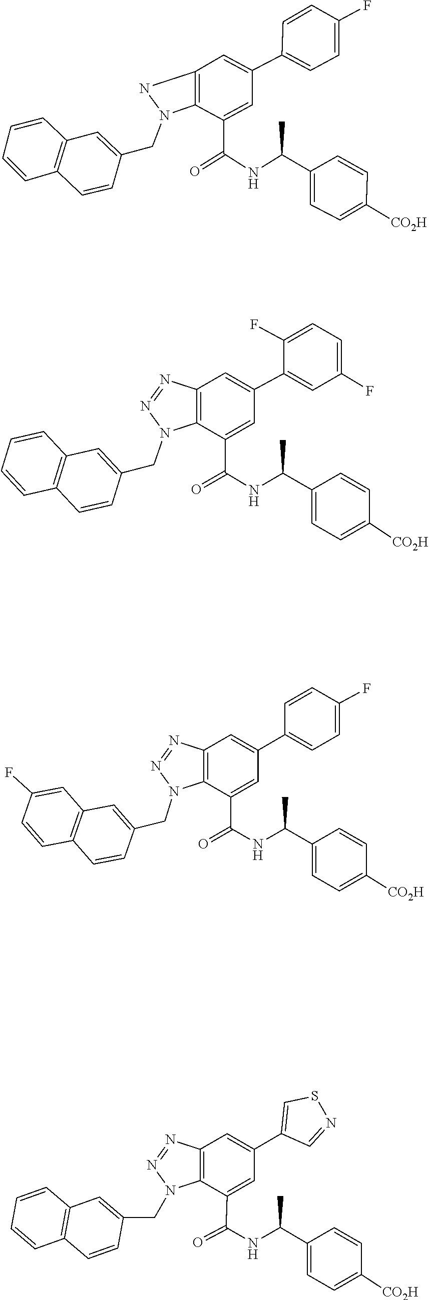

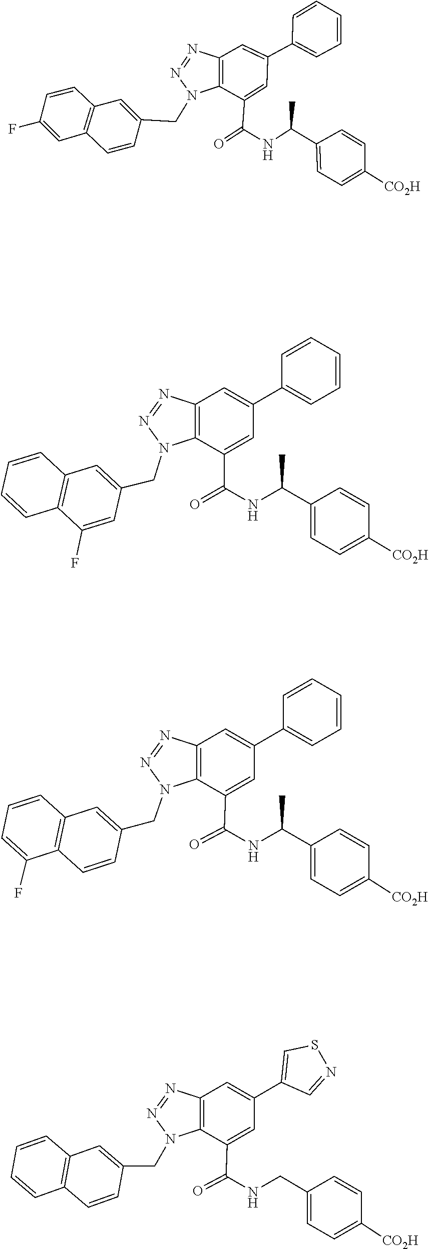 [SMILES: C[C@H](NC(=O)c1cc(-c2ccccc2)cc2nnn(Cc3ccc4cc(F)ccc4c3)c12)c1ccc(C(=O)O)cc1 ; C[C@H](NC(=O)c1cc(-c2ccccc2)cc2nnn(Cc3cc(F)c4ccccc4c3)c12)c1ccc(C(=O)O)cc1 ; C[C@H](NC(=O)c1cc(-c2ccccc2)cc2nnn(Cc3ccc4c(F)cccc4c3)c12)c1ccc(C(=O)O)cc1 ; O=C(O)c1ccc(CNC(=O)c2cc(-c3cnsc3)cc3nnn(Cc4ccc5ccccc5c4)c23)cc1]

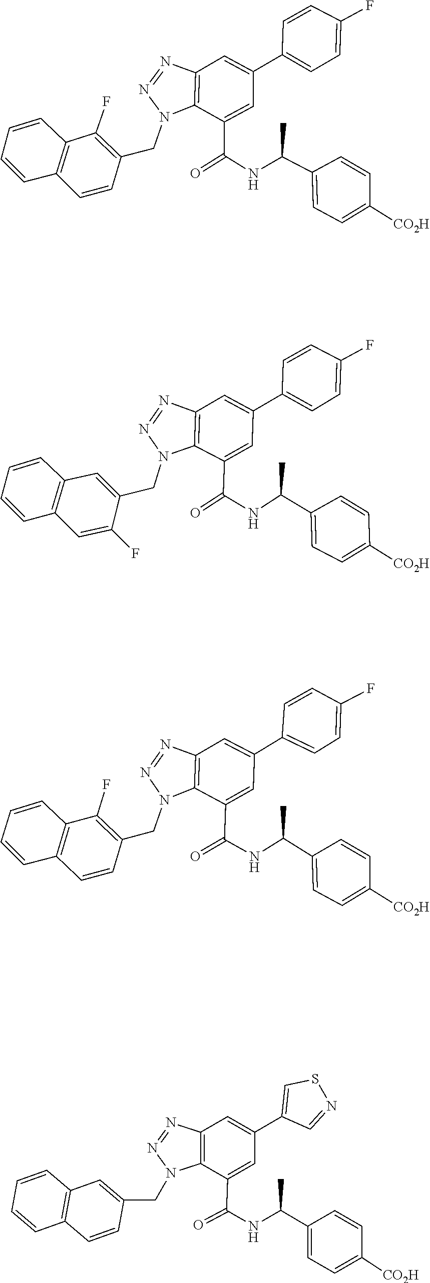 [SMILES: C[C@H](NC(=O)c1cc(-c2ccc(F)cc2)cc2nnn(Cc3ccc4ccccc4c3F)c12)c1ccc(C(=O)O)cc1 ; C[C@H](NC(=O)c1cc(-c2ccc(F)cc2)cc2nnn(Cc3cc4ccccc4cc3F)c12)c1ccc(C(=O)O)cc1 ; C[C@H](NC(=O)c1cc(-c2ccc(F)cc2)cc2nnn(Cc3ccc4ccccc4c3F)c12)c1ccc(C(=O)O)cc1 ; C[C@H](NC(=O)c1cc(-c2cnsc2)cc2nnn(Cc3ccc4ccccc4c3)c12)c1ccc(C(=O)O)cc1]

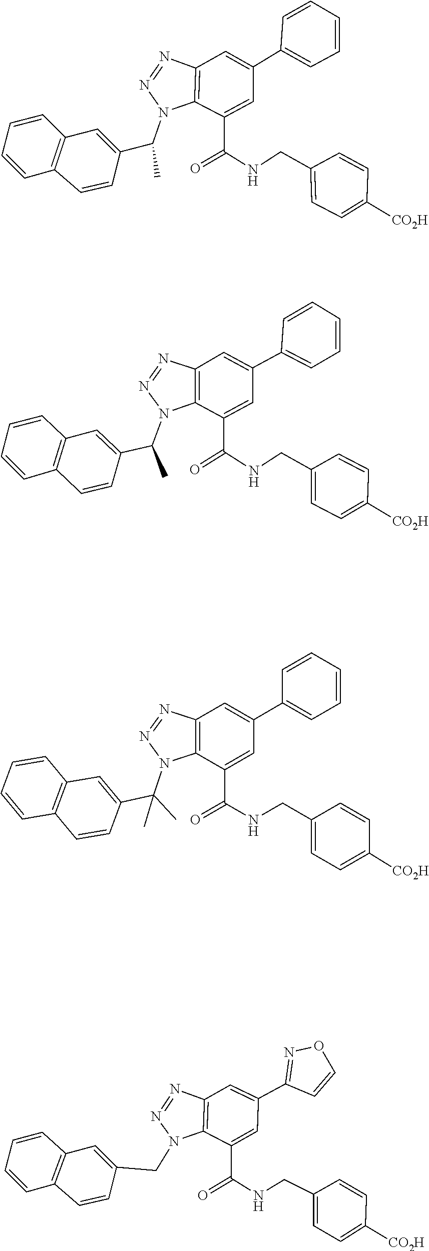 [SMILES: C[C@H](c1ccc2ccccc2c1)n1nnc2cc(-c3ccccc3)cc(C(=O)NCc3ccc(C(=O)O)cc3)c21 ; C[C@@H](c1ccc2ccccc2c1)n1nnc2cc(-c3ccccc3)cc(C(=O)NCc3ccc(C(=O)O)cc3)c21 ; CC(C)(c1ccc2ccccc2c1)n1nnc2cc(-c3ccccc3)cc(C(=O)NCc3ccc(C(=O)O)cc3)c21 ; O=C(O)c1ccc(CNC(=O)c2cc(-c3ccon3)cc3nnn(Cc4ccc5ccccc5c4)c23)cc1]

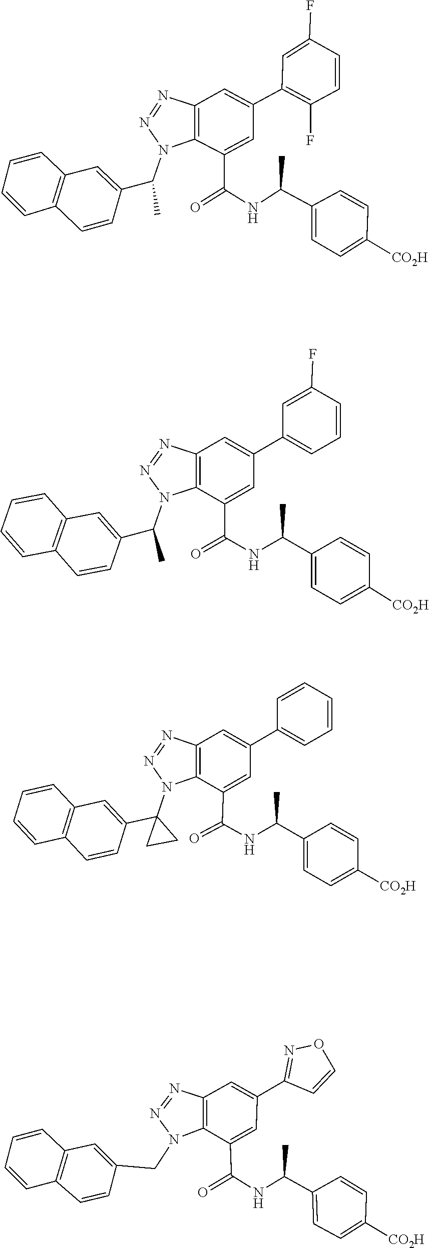 [SMILES: C[C@H](NC(=O)c1cc(-c2cc(F)ccc2F)cc2nnn([C@H](C)c3ccc4ccccc4c3)c12)c1ccc(C(=O)O)cc1 ; C[C@H](NC(=O)c1cc(-c2cccc(F)c2)cc2nnn([C@@H](C)c3ccc4ccccc4c3)c12)c1ccc(C(=O)O)cc1 ; C[C@H](NC(=O)c1cc(-c2ccccc2)cc2nnn(C3(c4ccc5ccccc5c4)CC3)c12)c1ccc(C(=O)O)cc1 ; C[C@H](NC(=O)c1cc(-c2ccon2)cc2nnn(Cc3ccc4ccccc4c3)c12)c1ccc(C(=O)O)cc1]

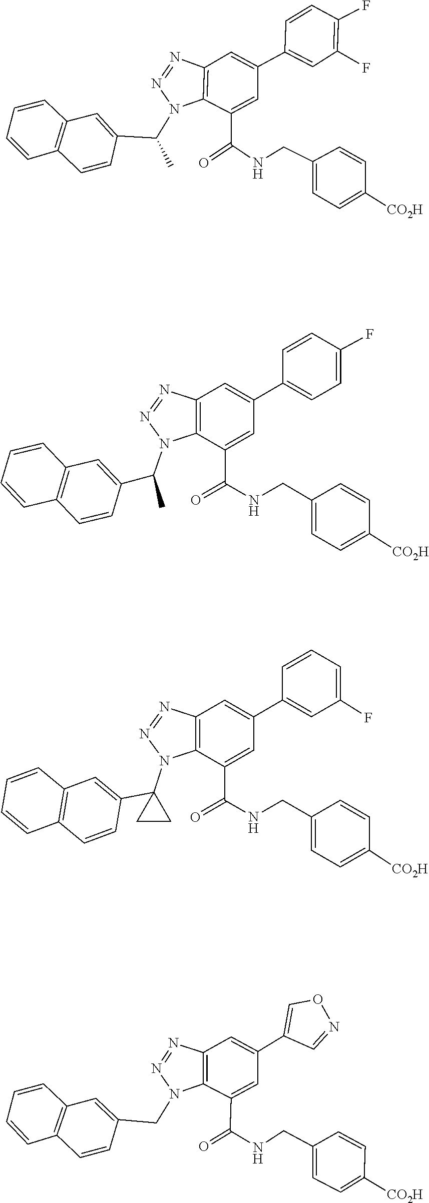 [SMILES: C[C@H](c1ccc2ccccc2c1)n1nnc2cc(-c3ccc(F)c(F)c3)cc(C(=O)NCc3ccc(C(=O)O)cc3)c21 ; C[C@@H](c1ccc2ccccc2c1)n1nnc2cc(-c3ccc(F)cc3)cc(C(=O)NCc3ccc(C(=O)O)cc3)c21 ; O=C(O)c1ccc(CNC(=O)c2cc(-c3cccc(F)c3)cc3nnn(C4(c5ccc6ccccc6c5)CC4)c23)cc1 ; O=C(O)c1ccc(CNC(=O)c2cc(-c3cnoc3)cc3nnn(Cc4ccc5ccccc5c4)c23)cc1]

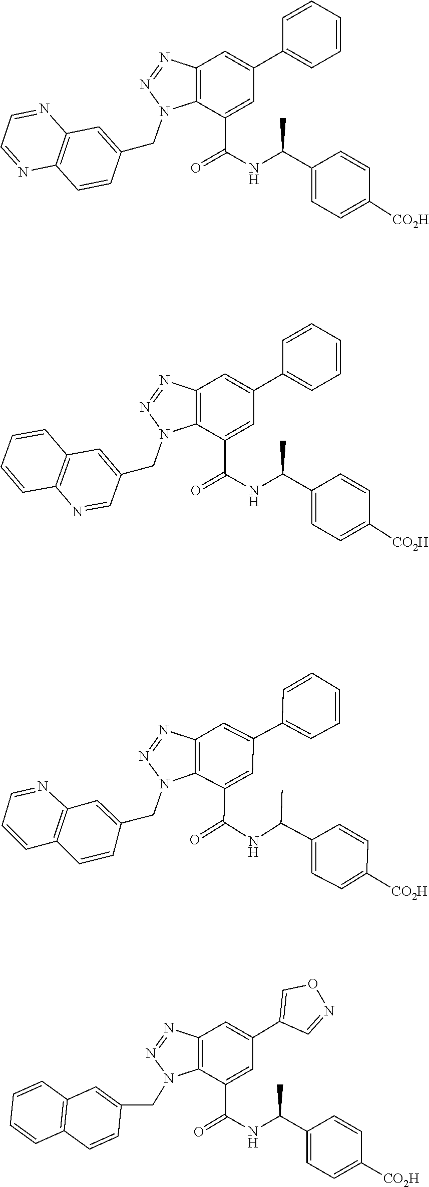 [SMILES: C[C@H](NC(=O)c1cc(-c2ccccc2)cc2nnn(Cc3ccc4nccnc4c3)c12)c1ccc(C(=O)O)cc1 ; C[C@H](NC(=O)c1cc(-c2ccccc2)cc2nnn(Cc3cnc4ccccc4c3)c12)c1ccc(C(=O)O)cc1 ; CC(NC(=O)c1cc(-c2ccccc2)cc2nnn(Cc3ccc4cccnc4c3)c12)c1ccc(C(=O)O)cc1 ; C[C@H](NC(=O)c1cc(-c2cnoc2)cc2nnn(Cc3ccc4ccccc4c3)c12)c1ccc(C(=O)O)cc1]

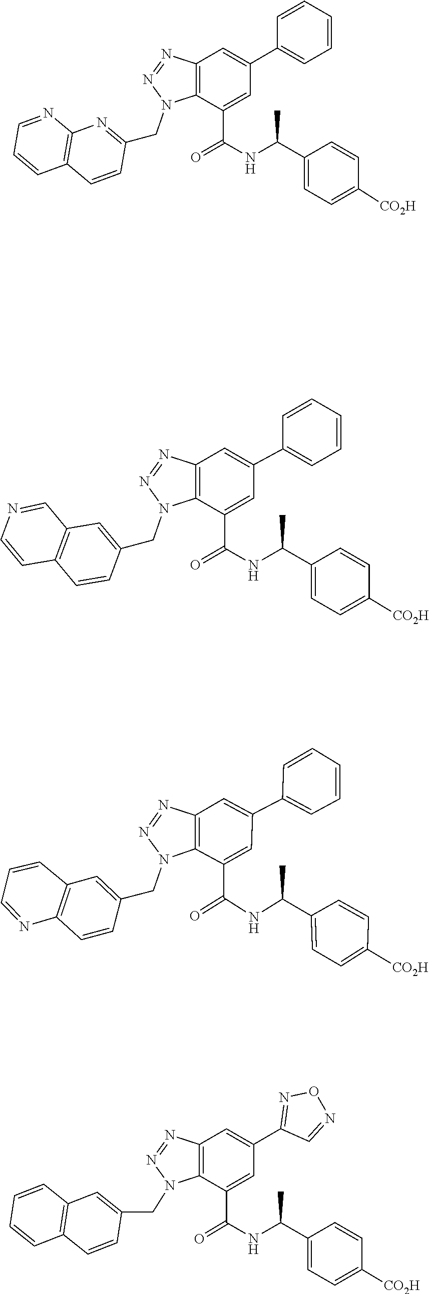 [SMILES: C[C@H](NC(=O)c1cc(-c2ccccc2)cc2nnn(Cc3ccc4cccnc4n3)c12)c1ccc(C(=O)O)cc1 ; C[C@H](NC(=O)c1cc(-c2ccccc2)cc2nnn(Cc3ccc4ccncc4c3)c12)c1ccc(C(=O)O)cc1 ; C[C@H](NC(=O)c1cc(-c2ccccc2)cc2nnn(Cc3ccc4ncccc4c3)c12)c1ccc(C(=O)O)cc1 ; C[C@H](NC(=O)c1cc(-c2cnon2)cc2nnn(Cc3ccc4ccccc4c3)c12)c1ccc(C(=O)O)cc1]

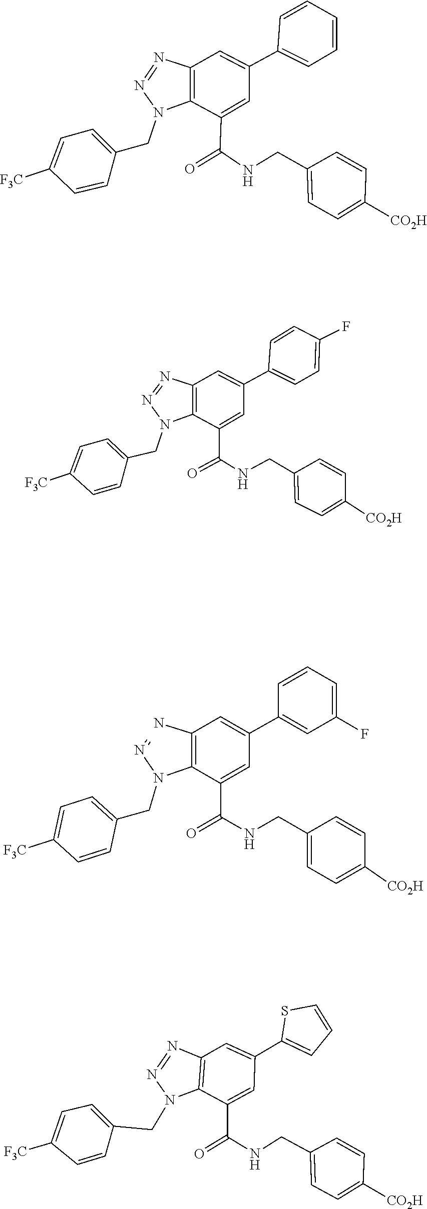

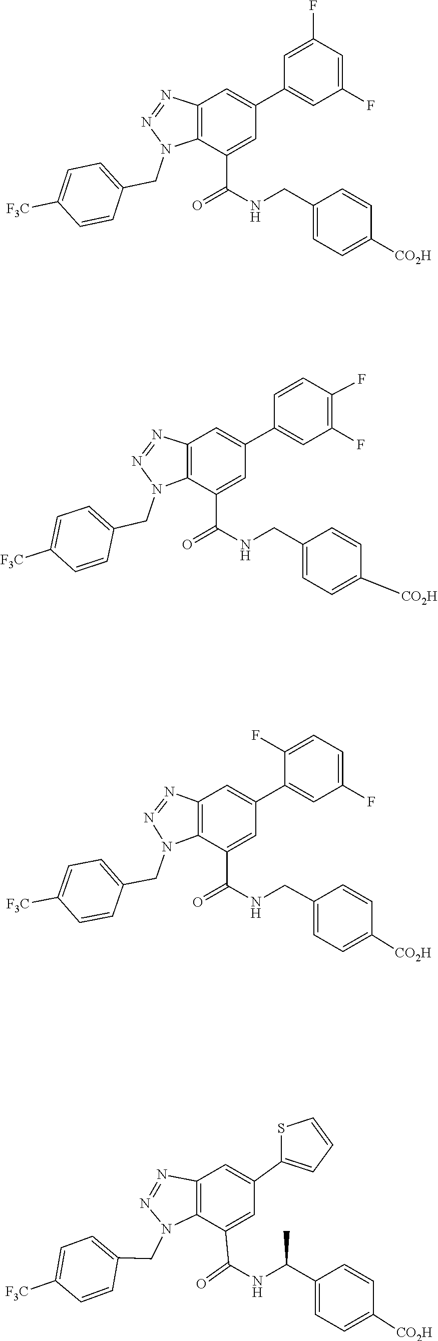 [SMILES: O=C(O)c1ccc(CNC(=O)c2cc(-c3cc(F)cc(F)c3)cc3nnn(Cc4ccc(C(F)(F)F)cc4)c23)cc1 ; O=C(O)c1ccc(CNC(=O)c2cc(-c3ccc(F)c(F)c3)cc3nnn(Cc4ccc(C(F)(F)F)cc4)c23)cc1 ; O=C(O)c1ccc(CNC(=O)c2cc(-c3cc(F)ccc3F)cc3nnn(Cc4ccc(C(F)(F)F)cc4)c23)cc1 ; C[C@H](NC(=O)c1cc(-c2cccs2)cc2nnn(Cc3ccc(C(F)(F)F)cc3)c12)c1ccc(C(=O)O)cc1]

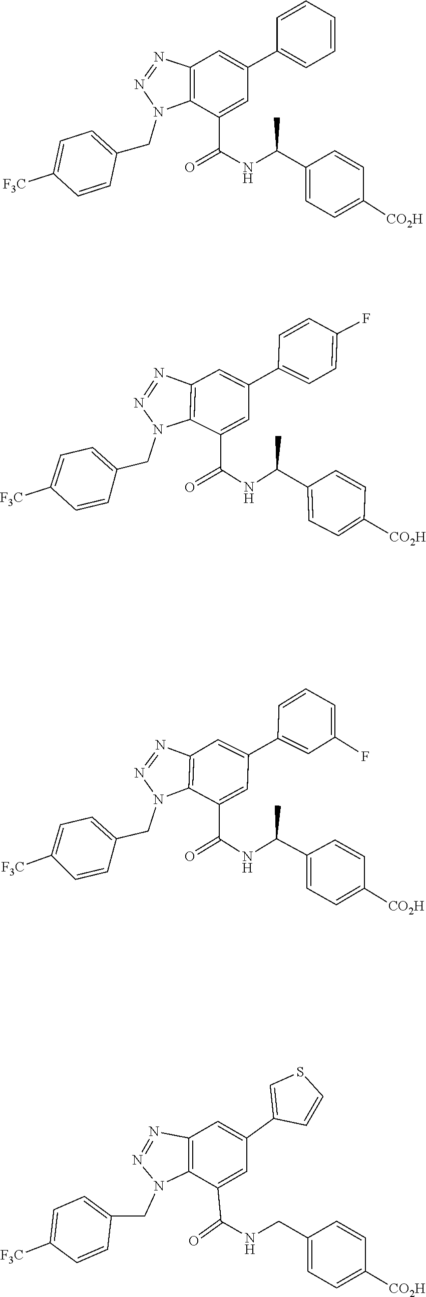 [SMILES: C[C@H](NC(=O)c1cc(-c2ccccc2)cc2nnn(Cc3ccc(C(F)(F)F)cc3)c12)c1ccc(C(=O)O)cc1 ; C[C@H](NC(=O)c1cc(-c2ccc(F)cc2)cc2nnn(Cc3ccc(C(F)(F)F)cc3)c12)c1ccc(C(=O)O)cc1 ; C[C@H](NC(=O)c1cc(-c2cccc(F)c2)cc2nnn(Cc3ccc(C(F)(F)F)cc3)c12)c1ccc(C(=O)O)cc1 ; O=C(O)c1ccc(CNC(=O)c2cc(-c3ccsc3)cc3nnn(Cc4ccc(C(F)(F)F)cc4)c23)cc1]

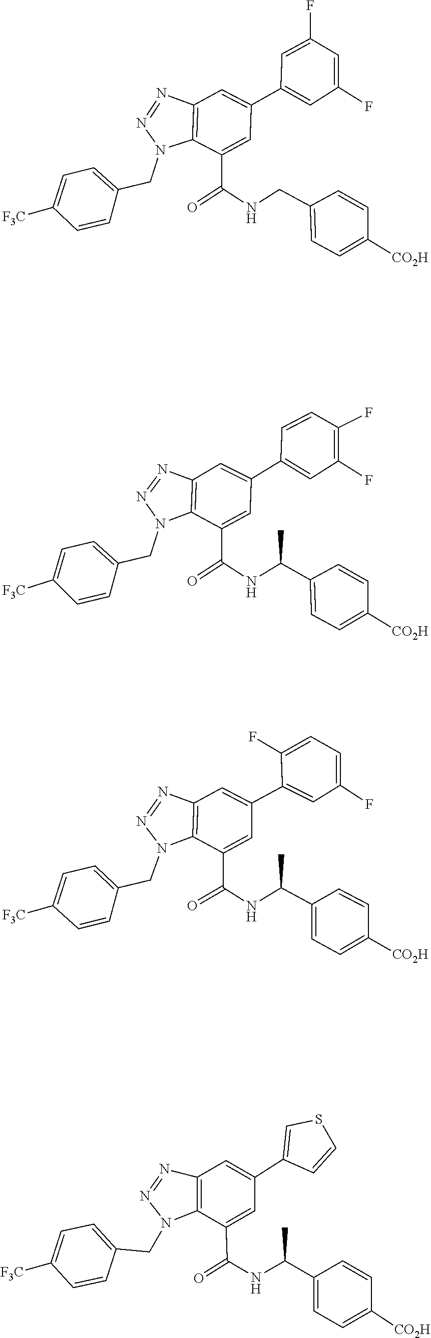 [SMILES: O=C(O)c1ccc(CNC(=O)c2cc(-c3cc(F)cc(F)c3)cc3nnn(Cc4ccc(C(F)(F)F)cc4)c23)cc1 ; C[C@H](NC(=O)c1cc(-c2ccc(F)c(F)c2)cc2nnn(Cc3ccc(C(F)(F)F)cc3)c12)c1ccc(C(=O)O)cc1 ; C[C@H](NC(=O)c1cc(-c2cc(F)ccc2F)cc2nnn(Cc3ccc(C(F)(F)F)cc3)c12)c1ccc(C(=O)O)cc1 ; C[C@H](NC(=O)c1cc(-c2ccsc2)cc2nnn(Cc3ccc(C(F)(F)F)cc3)c12)c1ccc(C(=O)O)cc1]

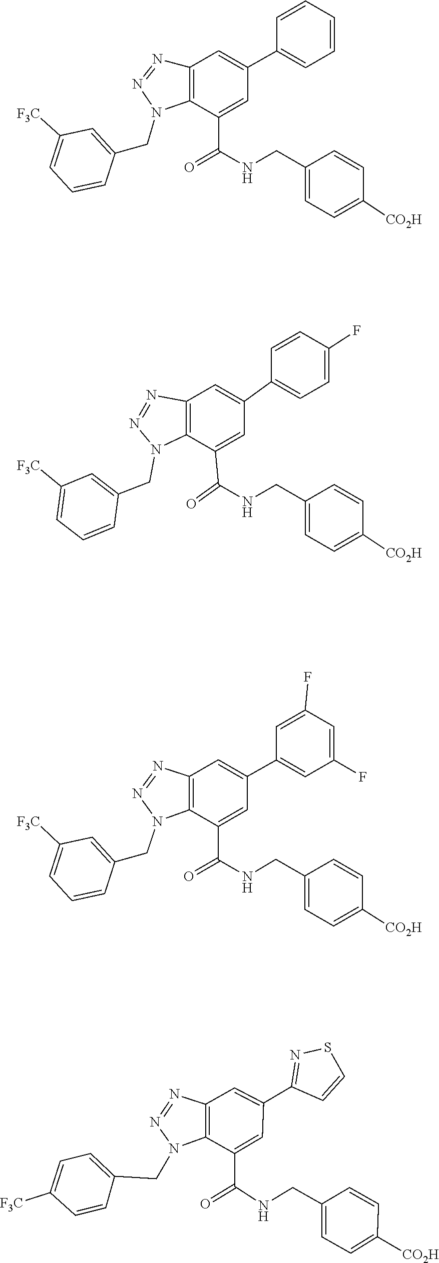 [SMILES: O=C(O)c1ccc(CNC(=O)c2cc(-c3ccccc3)cc3nnn(Cc4cccc(C(F)(F)F)c4)c23)cc1 ; O=C(O)c1ccc(CNC(=O)c2cc(-c3ccc(F)cc3)cc3nnn(Cc4cccc(C(F)(F)F)c4)c23)cc1 ; O=C(O)c1ccc(CNC(=O)c2cc(-c3cc(F)cc(F)c3)cc3nnn(Cc4cccc(C(F)(F)F)c4)c23)cc1 ; O=C(O)c1ccc(CNC(=O)c2cc(-c3ccsn3)cc3nnn(Cc4ccc(C(F)(F)F)cc4)c23)cc1]

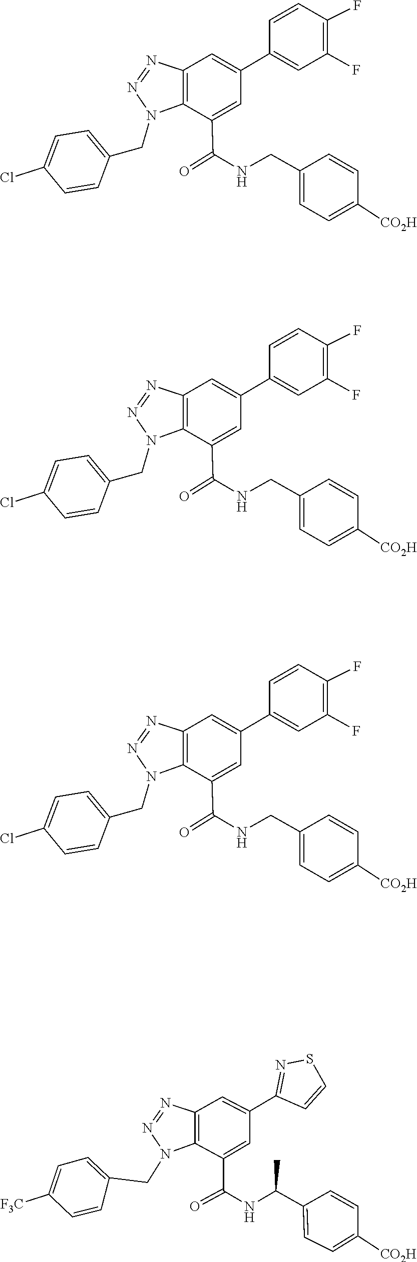

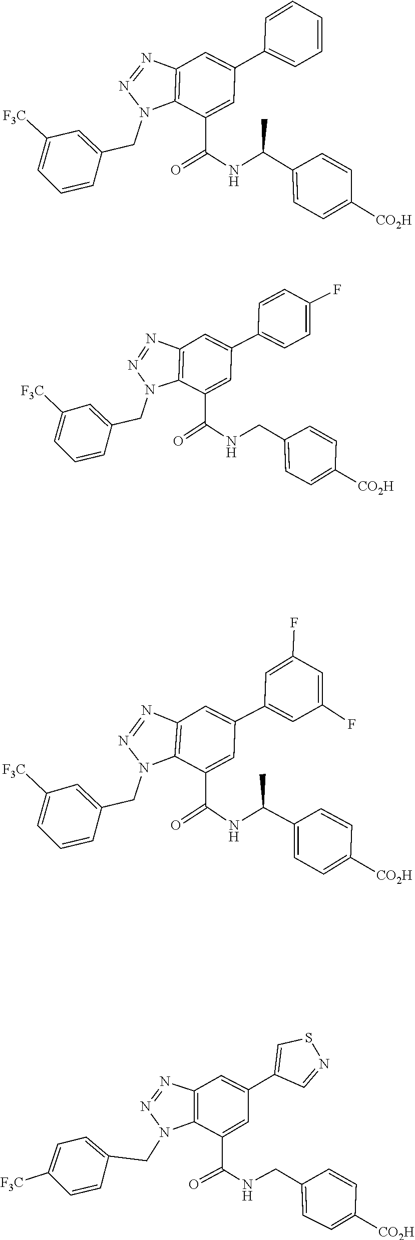 [SMILES: C[C@H](NC(=O)c1cc(-c2ccccc2)cc2nnn(Cc3cccc(C(F)(F)F)c3)c12)c1ccc(C(=O)O)cc1 ; O=C(O)c1ccc(CNC(=O)c2cc(-c3ccc(F)cc3)cc3nnn(Cc4cccc(C(F)(F)F)c4)c23)cc1 ; C[C@H](NC(=O)c1cc(-c2cc(F)cc(F)c2)cc2nnn(Cc3cccc(C(F)(F)F)c3)c12)c1ccc(C(=O)O)cc1 ; O=C(O)c1ccc(CNC(=O)c2cc(-c3cnsc3)cc3nnn(Cc4ccc(C(F)(F)F)cc4)c23)cc1]

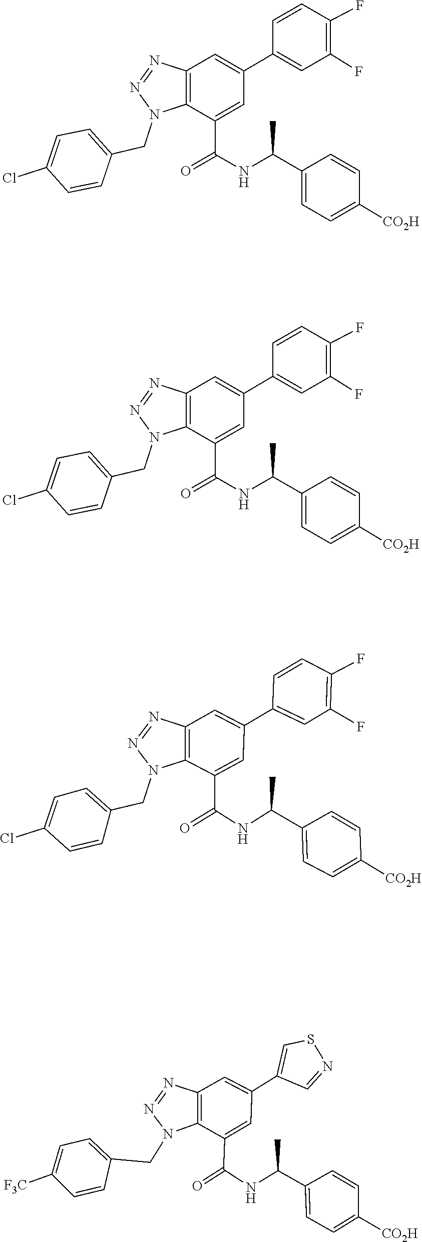

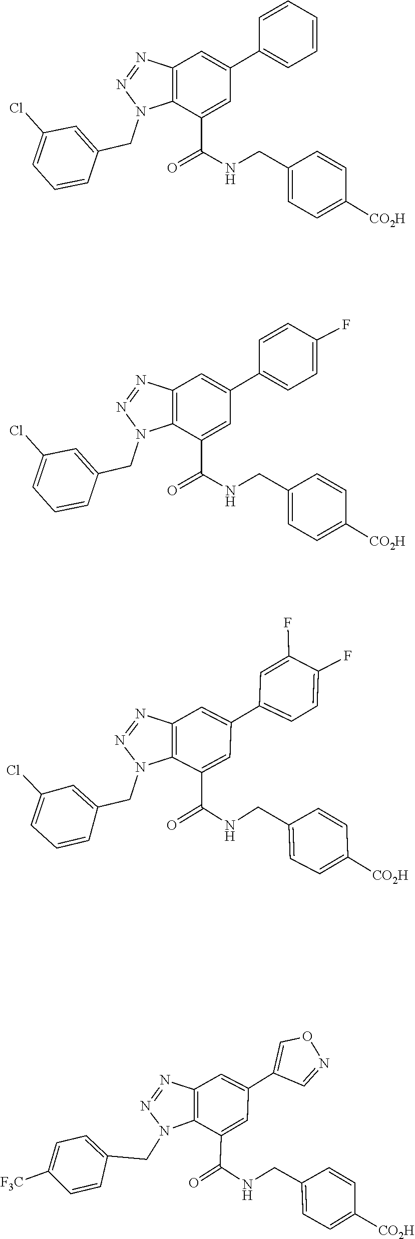 [SMILES: O=C(O)c1ccc(CNC(=O)c2cc(-c3ccccc3)cc3nnn(Cc4cccc(Cl)c4)c23)cc1 ; O=C(O)c1ccc(CNC(=O)c2cc(-c3ccc(F)cc3)cc3nnn(Cc4cccc(Cl)c4)c23)cc1 ; O=C(O)c1ccc(CNC(=O)c2cc(-c3ccc(F)c(F)c3)cc3nnn(Cc4cccc(Cl)c4)c23)cc1 ; O=C(O)c1ccc(CNC(=O)c2cc(-c3cnoc3)cc3nnn(Cc4ccc(C(F)(F)F)cc4)c23)cc1]

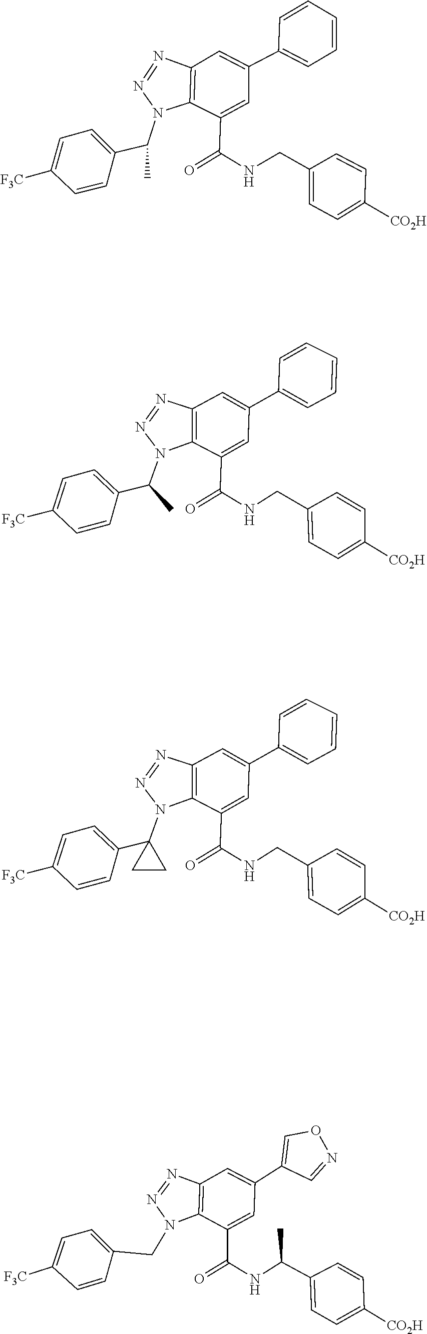 [SMILES: C[C@H](c1ccc(C(F)(F)F)cc1)n1nnc2cc(-c3ccccc3)cc(C(=O)NCc3ccc(C(=O)O)cc3)c21 ; C[C@@H](c1ccc(C(F)(F)F)cc1)n1nnc2cc(-c3ccccc3)cc(C(=O)NCc3ccc(C(=O)O)cc3)c21 ; O=C(O)c1ccc(CNC(=O)c2cc(-c3ccccc3)cc3nnn(C4(c5ccc(C(F)(F)F)cc5)CC4)c23)cc1 ; C[C@H](NC(=O)c1cc(-c2cnoc2)cc2nnn(Cc3ccc(C(F)(F)F)cc3)c12)c1ccc(C(=O)O)cc1]

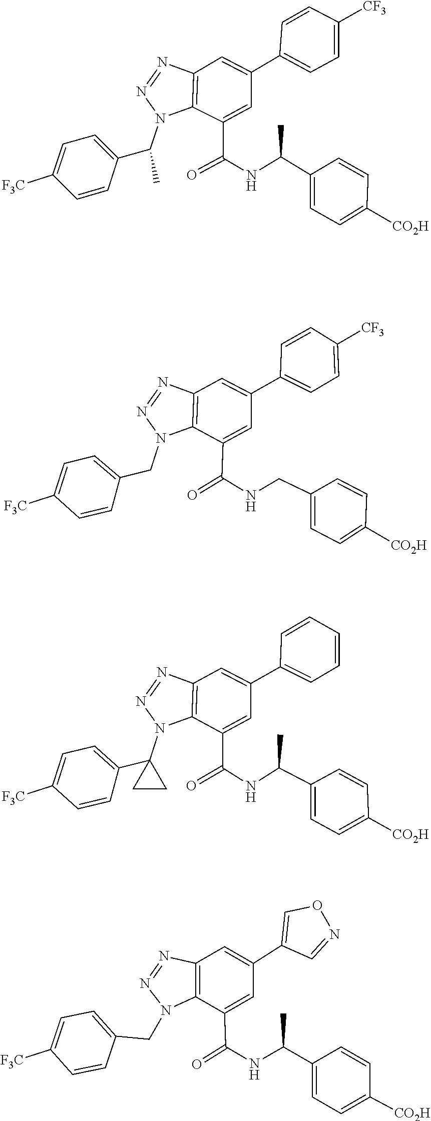 [SMILES: C[C@H](NC(=O)c1cc(-c2ccc(C(F)(F)F)cc2)cc2nnn([C@H](C)c3ccc(C(F)(F)F)cc3)c12)c1ccc(C(=O)O)cc1 ; O=C(O)c1ccc(CNC(=O)c2cc(-c3ccc(C(F)(F)F)cc3)cc3nnn(Cc4ccc(C(F)(F)F)cc4)c23)cc1 ; C[C@H](NC(=O)c1cc(-c2ccccc2)cc2nnn(C3(c4ccc(C(F)(F)F)cc4)CC3)c12)c1ccc(C(=O)O)cc1 ; C[C@H](NC(=O)c1cc(-c2cnoc2)cc2nnn(Cc3ccc(C(F)(F)F)cc3)c12)c1ccc(C(=O)O)cc1]

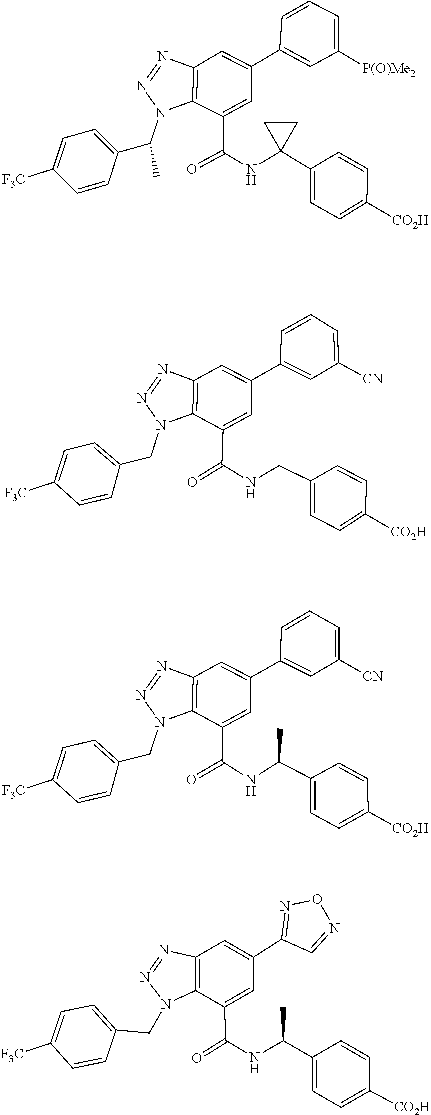

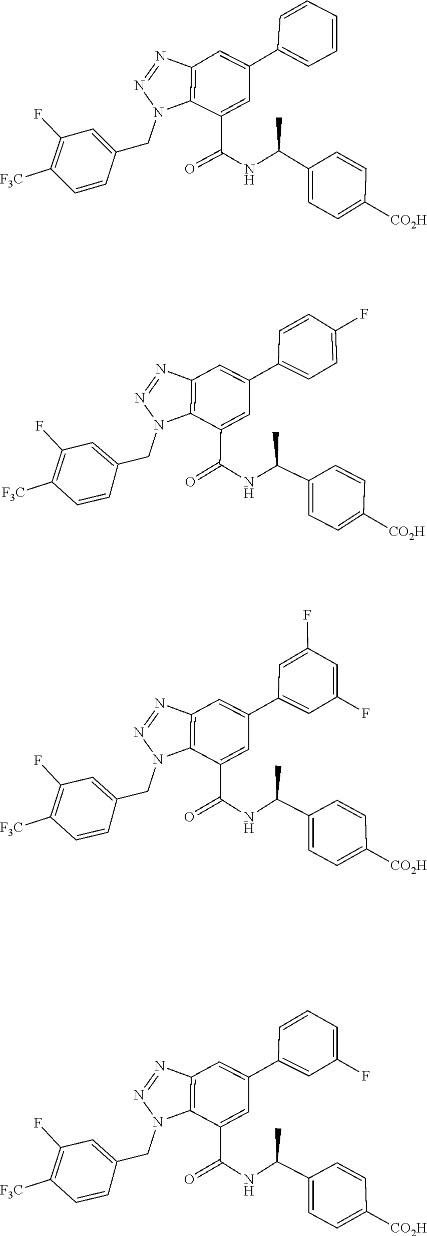 [SMILES: C[C@H](NC(=O)c1cc(-c2ccccc2)cc2nnn(Cc3ccc(C(F)(F)F)c(F)c3)c12)c1ccc(C(=O)O)cc1 ; C[C@H](NC(=O)c1cc(-c2ccc(F)cc2)cc2nnn(Cc3ccc(C(F)(F)F)c(F)c3)c12)c1ccc(C(=O)O)cc1 ; C[C@H](NC(=O)c1cc(-c2cc(F)cc(F)c2)cc2nnn(Cc3ccc(C(F)(F)F)c(F)c3)c12)c1ccc(C(=O)O)cc1 ; C[C@H](NC(=O)c1cc(-c2cccc(F)c2)cc2nnn(Cc3ccc(C(F)(F)F)c(F)c3)c12)c1ccc(C(=O)O)cc1]

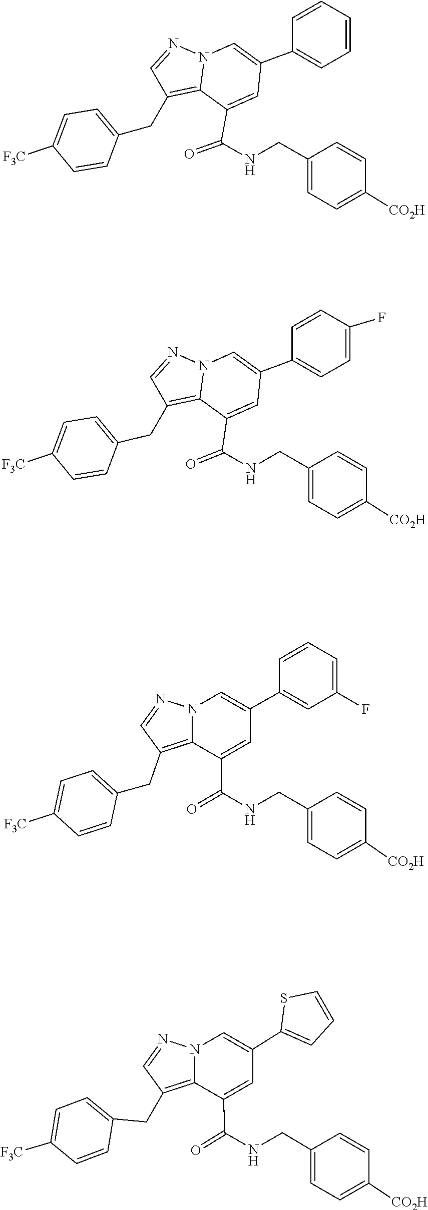 [SMILES: O=C(O)c1ccc(CNC(=O)c2cc(-c3ccccc3)cn3ncc(Cc4ccc(C(F)(F)F)cc4)c23)cc1 ; O=C(O)c1ccc(CNC(=O)c2cc(-c3ccc(F)cc3)cn3ncc(Cc4ccc(C(F)(F)F)cc4)c23)cc1 ; O=C(O)c1ccc(CNC(=O)c2cc(-c3cccc(F)c3)cn3ncc(Cc4ccc(C(F)(F)F)cc4)c23)cc1 ; O=C(O)c1ccc(CNC(=O)c2cc(-c3cccs3)cn3ncc(Cc4ccc(C(F)(F)F)cc4)c23)cc1]

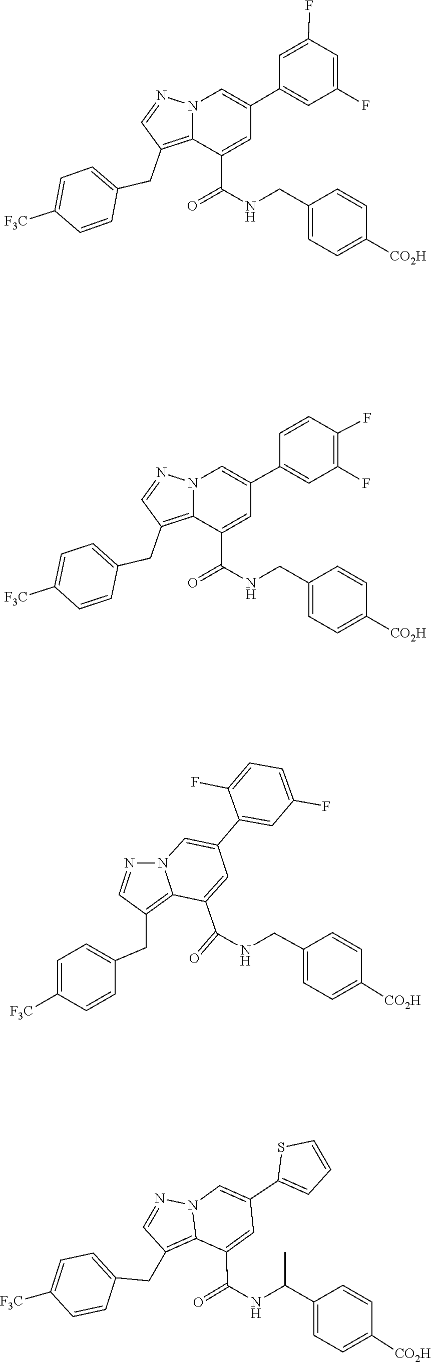 [SMILES: O=C(O)c1ccc(CNC(=O)c2cc(-c3cc(F)cc(F)c3)cn3ncc(Cc4ccc(C(F)(F)F)cc4)c23)cc1 ; O=C(O)c1ccc(CNC(=O)c2cc(-c3ccc(F)c(F)c3)cn3ncc(Cc4ccc(C(F)(F)F)cc4)c23)cc1 ; O=C(O)c1ccc(CNC(=O)c2cc(-c3cc(F)ccc3F)cn3ncc(Cc4ccc(C(F)(F)F)cc4)c23)cc1 ; CC(NC(=O)c1cc(-c2cccs2)cn2ncc(Cc3ccc(C(F)(F)F)cc3)c12)c1ccc(C(=O)O)cc1]

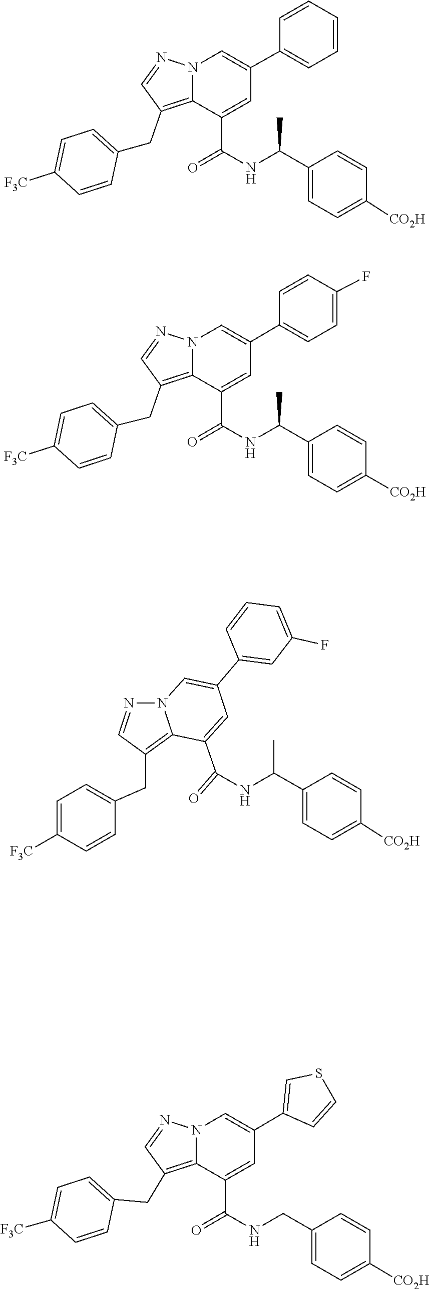 [SMILES: C[C@H](NC(=O)c1cc(-c2ccccc2)cn2ncc(Cc3ccc(C(F)(F)F)cc3)c12)c1ccc(C(=O)O)cc1 ; C[C@H](NC(=O)c1cc(-c2ccc(F)cc2)cn2ncc(Cc3ccc(C(F)(F)F)cc3)c12)c1ccc(C(=O)O)cc1 ; CC(NC(=O)c1cc(-c2cccc(F)c2)cn2ncc(Cc3ccc(C(F)(F)F)cc3)c12)c1ccc(C(=O)O)cc1 ; O=C(O)c1ccc(CNC(=O)c2cc(-c3ccsc3)cn3ncc(Cc4ccc(C(F)(F)F)cc4)c23)cc1]

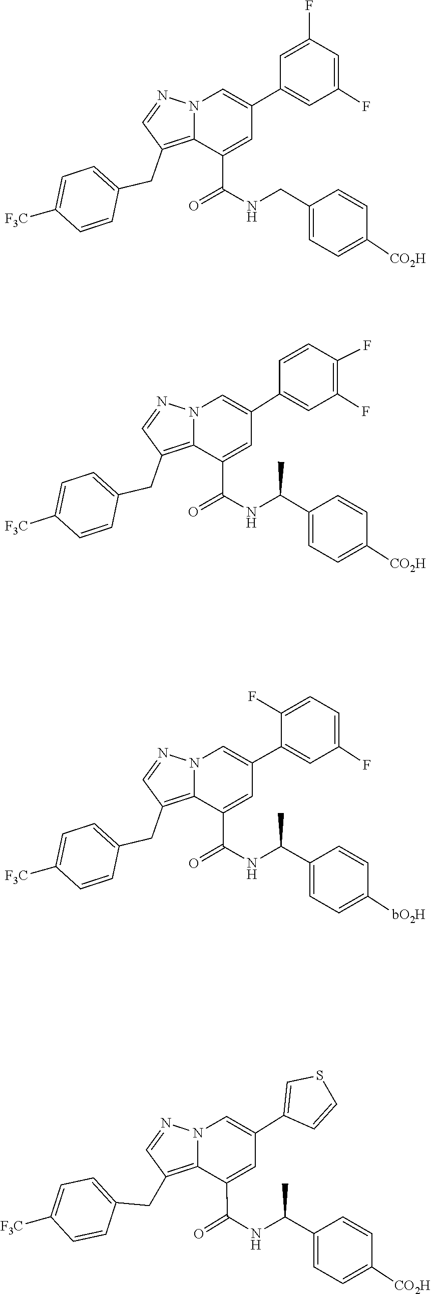

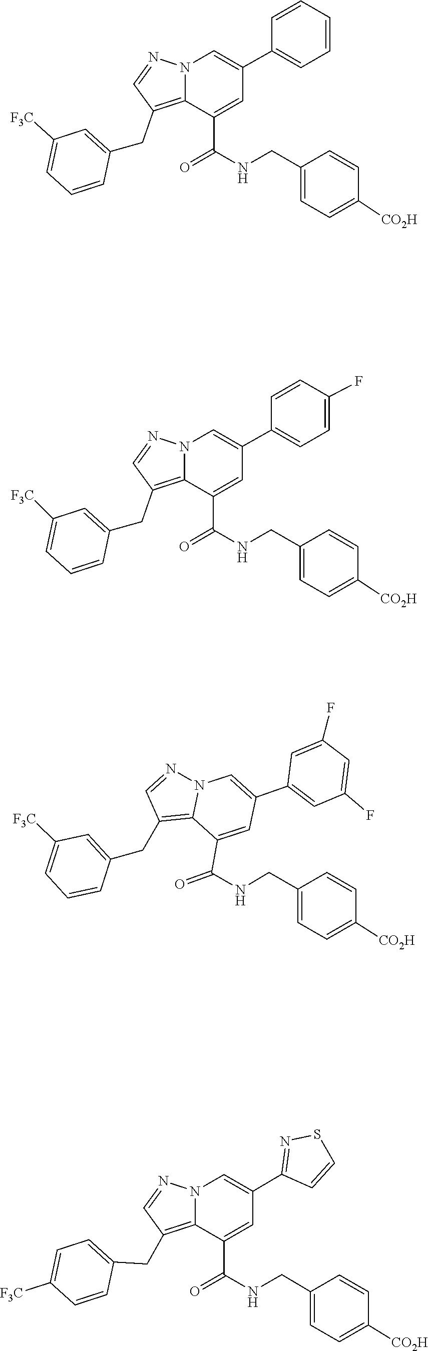 [SMILES: O=C(O)c1ccc(CNC(=O)c2cc(-c3ccccc3)cn3ncc(Cc4cccc(C(F)(F)F)c4)c23)cc1 ; O=C(O)c1ccc(CNC(=O)c2cc(-c3ccc(F)cc3)cn3ncc(Cc4cccc(C(F)(F)F)c4)c23)cc1 ; O=C(O)c1ccc(CNC(=O)c2cc(-c3cc(F)cc(F)c3)cn3ncc(Cc4cccc(C(F)(F)F)c4)c23)cc1 ; O=C(O)c1ccc(CNC(=O)c2cc(-c3ccsn3)cn3ncc(Cc4ccc(C(F)(F)F)cc4)c23)cc1]

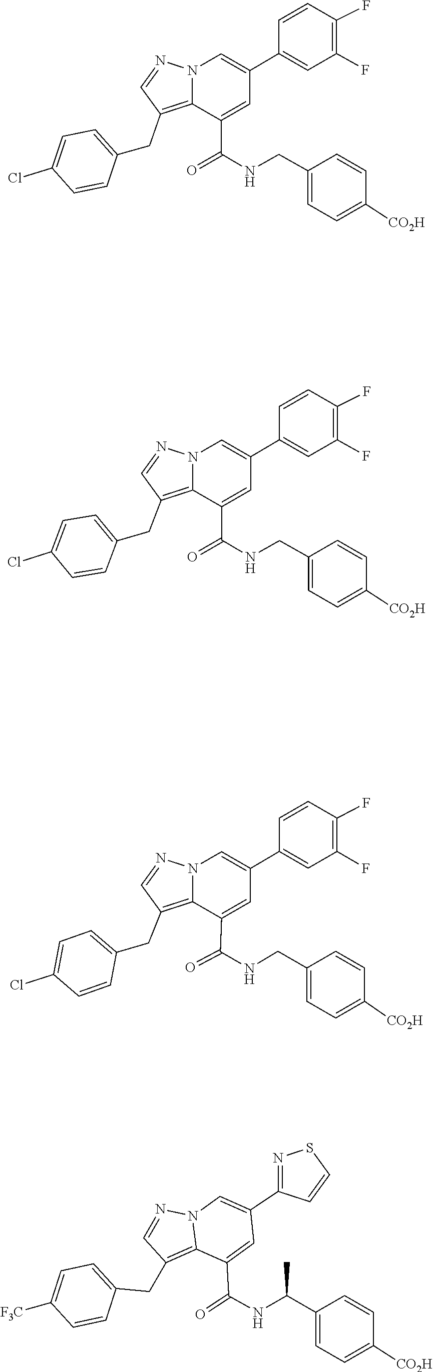 [SMILES: O=C(O)c1ccc(CNC(=O)c2cc(-c3ccc(F)c(F)c3)cn3ncc(Cc4ccc(Cl)cc4)c23)cc1 ; O=C(O)c1ccc(CNC(=O)c2cc(-c3ccc(F)c(F)c3)cn3ncc(Cc4ccc(Cl)cc4)c23)cc1 ; O=C(O)c1ccc(CNC(=O)c2cc(-c3ccc(F)c(F)c3)cn3ncc(Cc4ccc(Cl)cc4)c23)cc1 ; C[C@H](NC(=O)c1cc(-c2ccsn2)cn2ncc(Cc3ccc(C(F)(F)F)cc3)c12)c1ccc(C(=O)O)cc1]

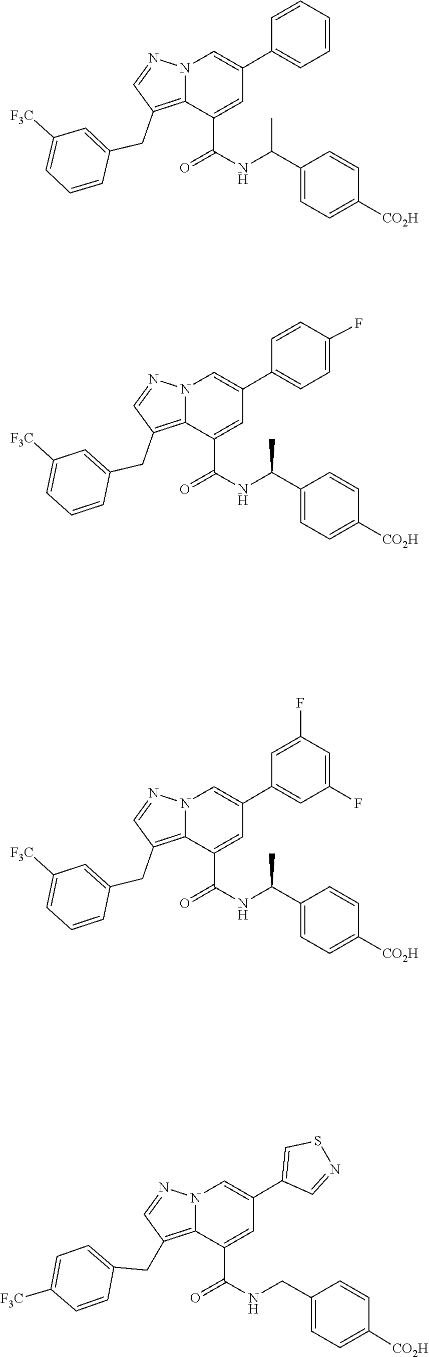 [SMILES: CC(NC(=O)c1cc(-c2ccccc2)cn2ncc(Cc3cccc(C(F)(F)F)c3)c12)c1ccc(C(=O)O)cc1 ; C[C@H](NC(=O)c1cc(-c2ccc(F)cc2)cn2ncc(Cc3cccc(C(F)(F)F)c3)c12)c1ccc(C(=O)O)cc1 ; C[C@H](NC(=O)c1cc(-c2cc(F)cc(F)c2)cn2ncc(Cc3cccc(C(F)(F)F)c3)c12)c1ccc(C(=O)O)cc1 ; O=C(O)c1ccc(CNC(=O)c2cc(-c3cnsc3)cn3ncc(Cc4ccc(C(F)(F)F)cc4)c23)cc1]

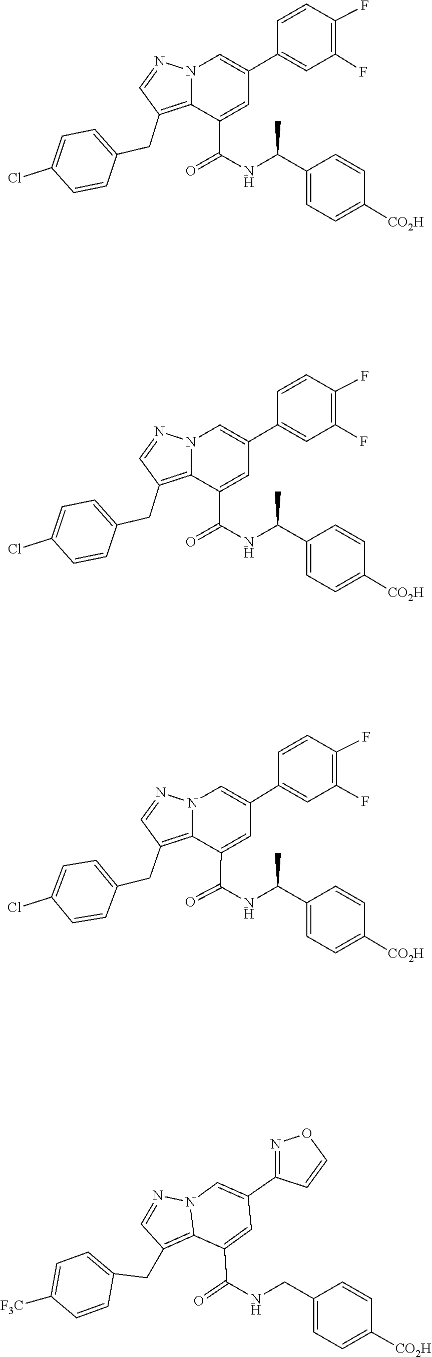 [SMILES: C[C@H](NC(=O)c1cc(-c2ccc(F)c(F)c2)cn2ncc(Cc3ccc(Cl)cc3)c12)c1ccc(C(=O)O)cc1 ; C[C@H](NC(=O)c1cc(-c2ccc(F)c(F)c2)cn2ncc(Cc3ccc(Cl)cc3)c12)c1ccc(C(=O)O)cc1 ; C[C@H](NC(=O)c1cc(-c2ccc(F)c(F)c2)cn2ncc(Cc3ccc(Cl)cc3)c12)c1ccc(C(=O)O)cc1 ; O=C(O)c1ccc(CNC(=O)c2cc(-c3ccon3)cn3ncc(Cc4ccc(C(F)(F)F)cc4)c23)cc1]

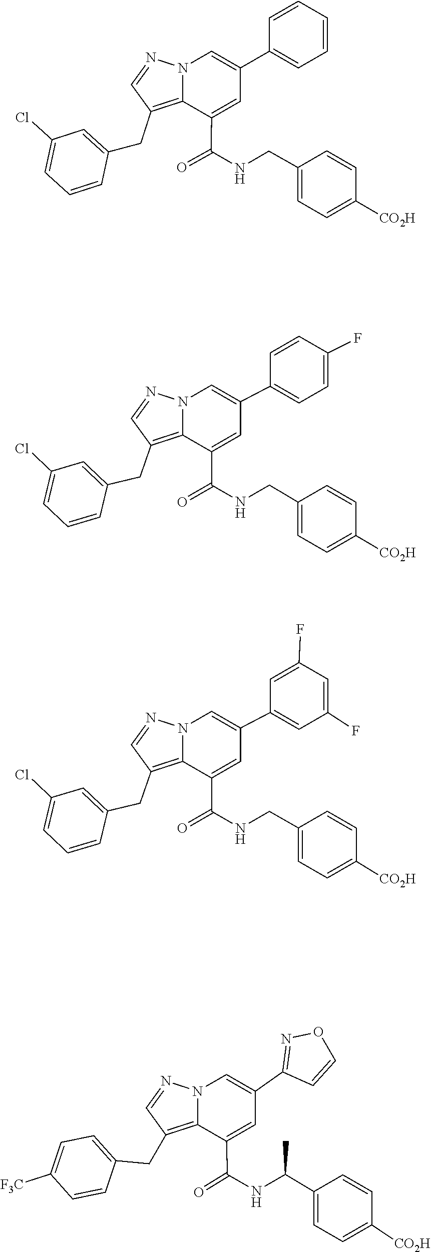 [SMILES: O=C(O)c1ccc(CNC(=O)c2cc(-c3ccccc3)cn3ncc(Cc4cccc(Cl)c4)c23)cc1 ; O=C(O)c1ccc(CNC(=O)c2cc(-c3ccc(F)cc3)cn3ncc(Cc4cccc(Cl)c4)c23)cc1 ; O=C(O)c1ccc(CNC(=O)c2cc(-c3cc(F)cc(F)c3)cn3ncc(Cc4cccc(Cl)c4)c23)cc1 ; C[C@H](NC(=O)c1cc(-c2ccon2)cn2ncc(Cc3ccc(C(F)(F)F)cc3)c12)c1ccc(C(=O)O)cc1]

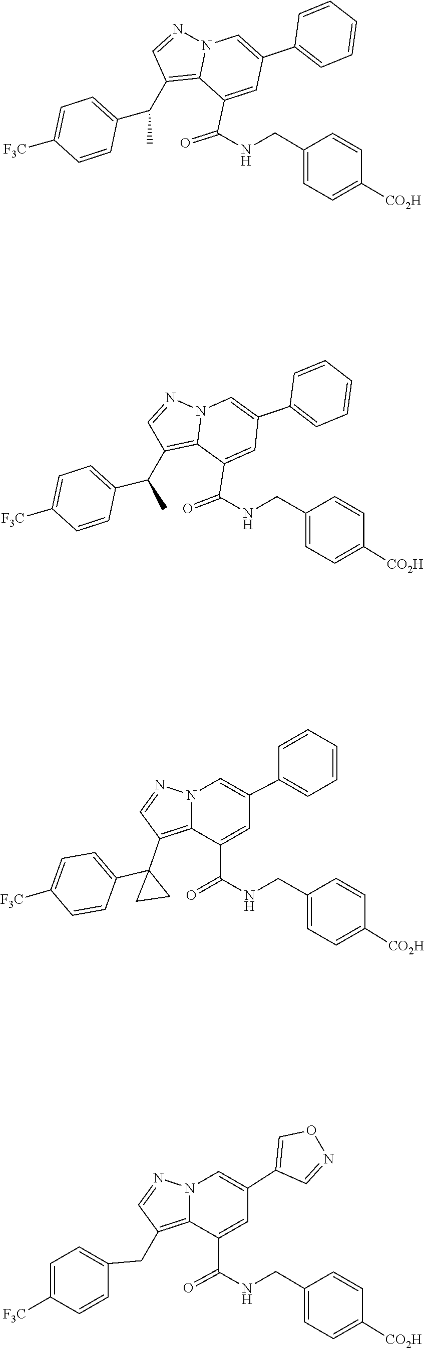 [SMILES: C[C@H](c1ccc(C(F)(F)F)cc1)c1cnn2cc(-c3ccccc3)cc(C(=O)NCc3ccc(C(=O)O)cc3)c12 ; C[C@@H](c1ccc(C(F)(F)F)cc1)c1cnn2cc(-c3ccccc3)cc(C(=O)NCc3ccc(C(=O)O)cc3)c12 ; O=C(O)c1ccc(CNC(=O)c2cc(-c3ccccc3)cn3ncc(C4(c5ccc(C(F)(F)F)cc5)CC4)c23)cc1 ; O=C(O)c1ccc(CNC(=O)c2cc(-c3cnoc3)cn3ncc(Cc4ccc(C(F)(F)F)cc4)c23)cc1]

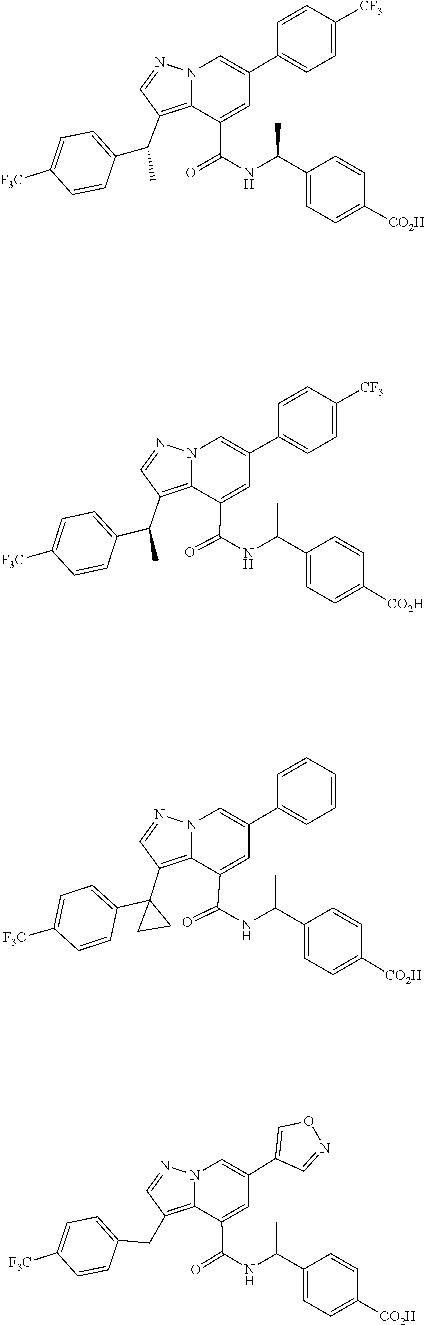 [SMILES: C[C@H](c1ccc(C(F)(F)F)cc1)c1cnn2cc(-c3ccc(C(F)(F)F)cc3)cc(C(=O)N[C@@H](C)c3ccc(C(=O)O)cc3)c12 ; CC(NC(=O)c1cc(-c2ccc(C(F)(F)F)cc2)cn2ncc([C@@H](C)c3ccc(C(F)(F)F)cc3)c12)c1ccc(C(=O)O)cc1 ; CC(NC(=O)c1cc(-c2ccccc2)cn2ncc(C3(c4ccc(C(F)(F)F)cc4)CC3)c12)c1ccc(C(=O)O)cc1 ; CC(NC(=O)c1cc(-c2cnoc2)cn2ncc(Cc3ccc(C(F)(F)F)cc3)c12)c1ccc(C(=O)O)cc1]

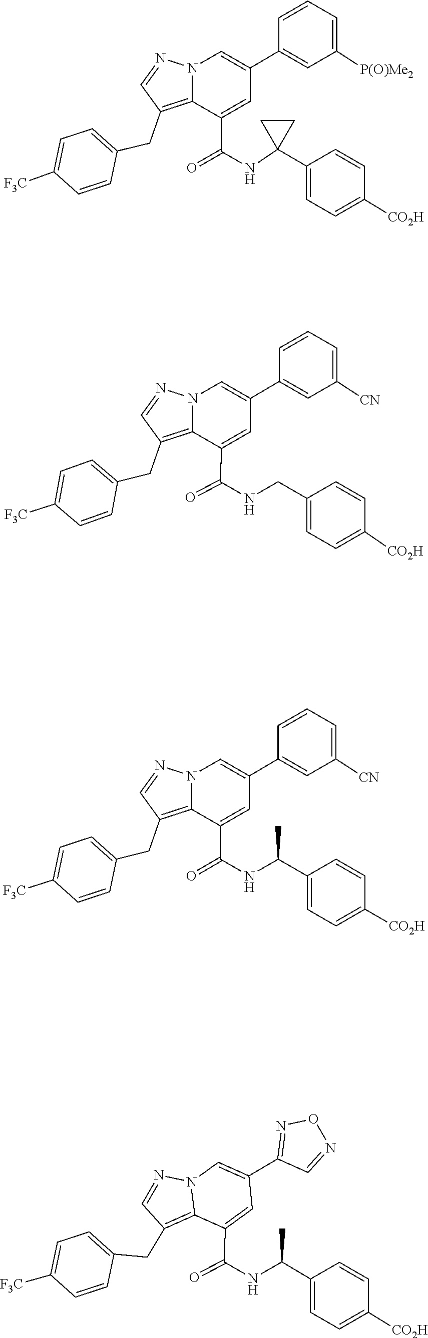

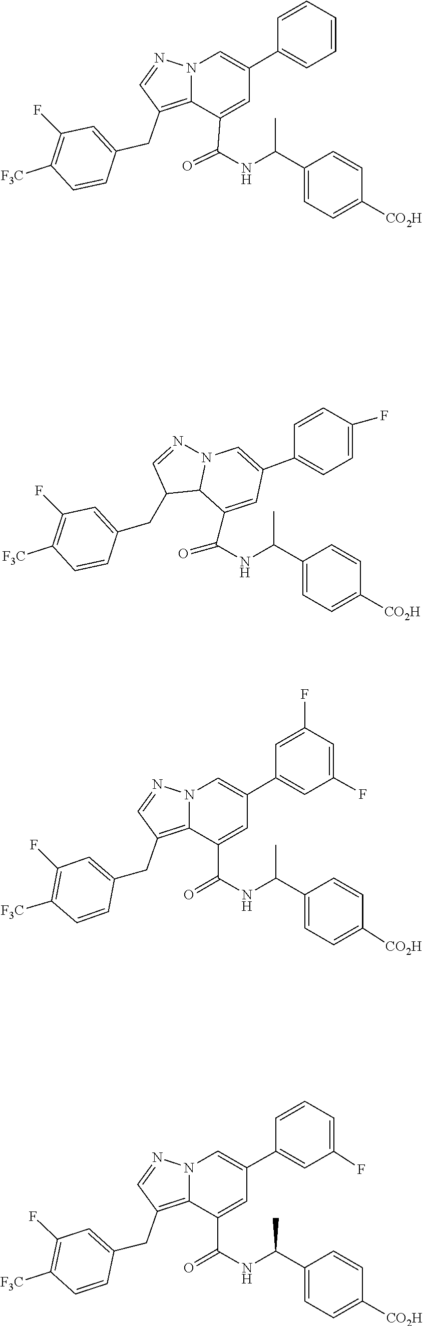 [SMILES: CC(NC(=O)c1cc(-c2ccccc2)cn2ncc(Cc3ccc(C(F)(F)F)c(F)c3)c12)c1ccc(C(=O)O)cc1 ; CC(NC(=O)C1=CC(c2ccc(F)cc2)=CN2N=CC(Cc3ccc(C(F)(F)F)c(F)c3)C12)c1ccc(C(=O)O)cc1 ; CC(NC(=O)c1cc(-c2cc(F)cc(F)c2)cn2ncc(Cc3ccc(C(F)(F)F)c(F)c3)c12)c1ccc(C(=O)O)cc1 ; C[C@H](NC(=O)c1cc(-c2cccc(F)c2)cn2ncc(Cc3ccc(C(F)(F)F)c(F)c3)c12)c1ccc(C(=O)O)cc1]

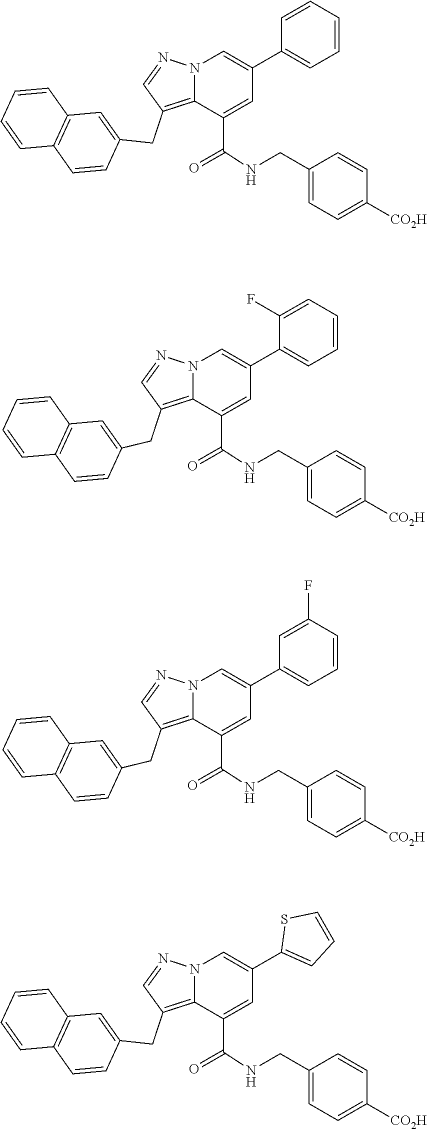 [SMILES: O=C(O)c1ccc(CNC(=O)c2cc(-c3ccccc3)cn3ncc(Cc4ccc5ccccc5c4)c23)cc1 ; O=C(O)c1ccc(CNC(=O)c2cc(-c3ccccc3F)cn3ncc(Cc4ccc5ccccc5c4)c23)cc1 ; O=C(O)c1ccc(CNC(=O)c2cc(-c3cccc(F)c3)cn3ncc(Cc4ccc5ccccc5c4)c23)cc1 ; O=C(O)c1ccc(CNC(=O)c2cc(-c3cccs3)cn3ncc(Cc4ccc5ccccc5c4)c23)cc1]

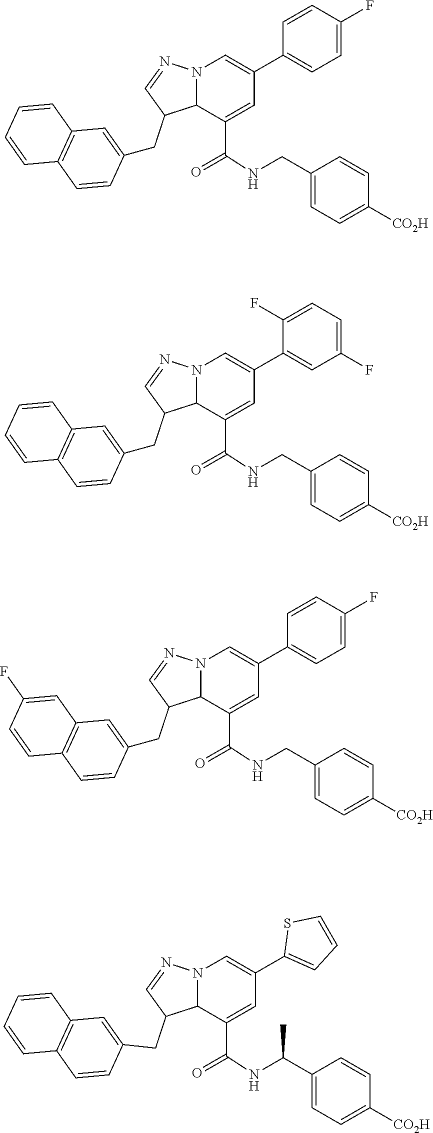 [SMILES: O=C(NCc1ccc(C(=O)O)cc1)C1=CC(c2ccc(F)cc2)=CN2N=CC(Cc3ccc4ccccc4c3)C12 ; O=C(NCc1ccc(C(=O)O)cc1)C1=CC(c2cc(F)ccc2F)=CN2N=CC(Cc3ccc4ccccc4c3)C12 ; O=C(NCc1ccc(C(=O)O)cc1)C1=CC(c2ccc(F)cc2)=CN2N=CC(Cc3ccc4ccc(F)cc4c3)C12 ; C[C@H](NC(=O)C1=CC(c2cccs2)=CN2N=CC(Cc3ccc4ccccc4c3)C12)c1ccc(C(=O)O)cc1]

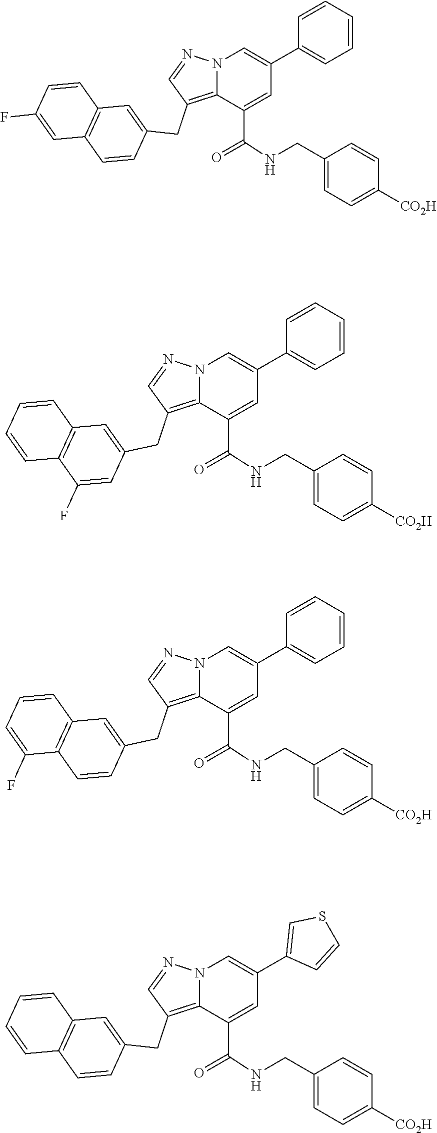 [SMILES: O=C(O)c1ccc(CNC(=O)c2cc(-c3ccccc3)cn3ncc(Cc4ccc5cc(F)ccc5c4)c23)cc1 ; O=C(O)c1ccc(CNC(=O)c2cc(-c3ccccc3)cn3ncc(Cc4cc(F)c5ccccc5c4)c23)cc1 ; O=C(O)c1ccc(CNC(=O)c2cc(-c3ccccc3)cn3ncc(Cc4ccc5c(F)cccc5c4)c23)cc1 ; O=C(O)c1ccc(CNC(=O)c2cc(-c3ccsc3)cn3ncc(Cc4ccc5ccccc5c4)c23)cc1]

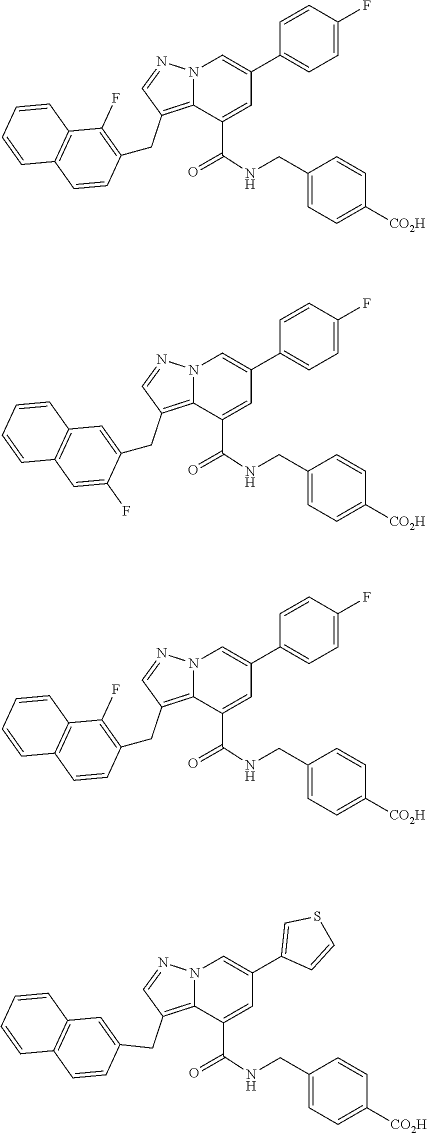 [SMILES: O=C(O)c1ccc(CNC(=O)c2cc(-c3ccc(F)cc3)cn3ncc(Cc4ccc5ccccc5c4F)c23)cc1 ; O=C(O)c1ccc(CNC(=O)c2cc(-c3ccc(F)cc3)cn3ncc(Cc4cc5ccccc5cc4F)c23)cc1 ; O=C(O)c1ccc(CNC(=O)c2cc(-c3ccc(F)cc3)cn3ncc(Cc4ccc5ccccc5c4F)c23)cc1 ; O=C(O)c1ccc(CNC(=O)c2cc(-c3ccsc3)cn3ncc(Cc4ccc5ccccc5c4)c23)cc1]

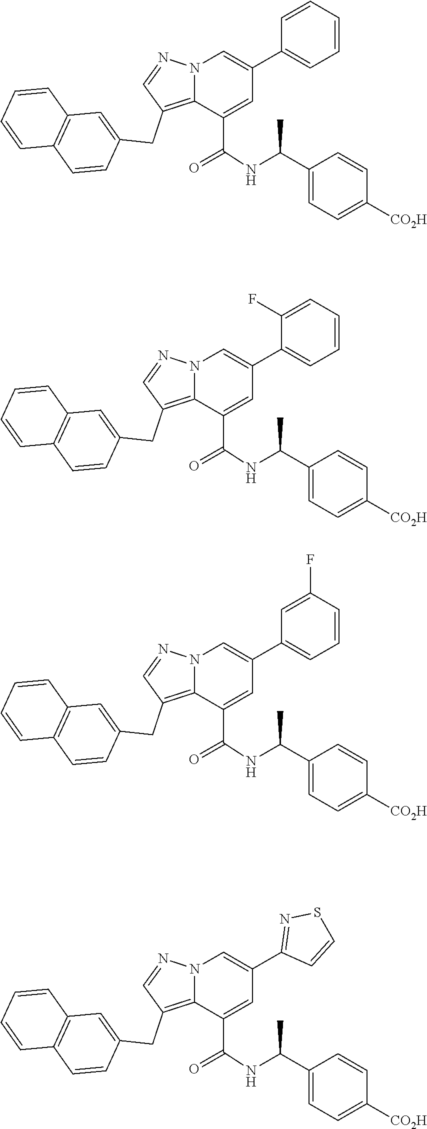 [SMILES: C[C@H](NC(=O)c1cc(-c2ccccc2)cn2ncc(Cc3ccc4ccccc4c3)c12)c1ccc(C(=O)O)cc1 ; C[C@H](NC(=O)c1cc(-c2ccccc2F)cn2ncc(Cc3ccc4ccccc4c3)c12)c1ccc(C(=O)O)cc1 ; C[C@H](NC(=O)c1cc(-c2cccc(F)c2)cn2ncc(Cc3ccc4ccccc4c3)c12)c1ccc(C(=O)O)cc1 ; C[C@H](NC(=O)c1cc(-c2ccsn2)cn2ncc(Cc3ccc4ccccc4c3)c12)c1ccc(C(=O)O)cc1]

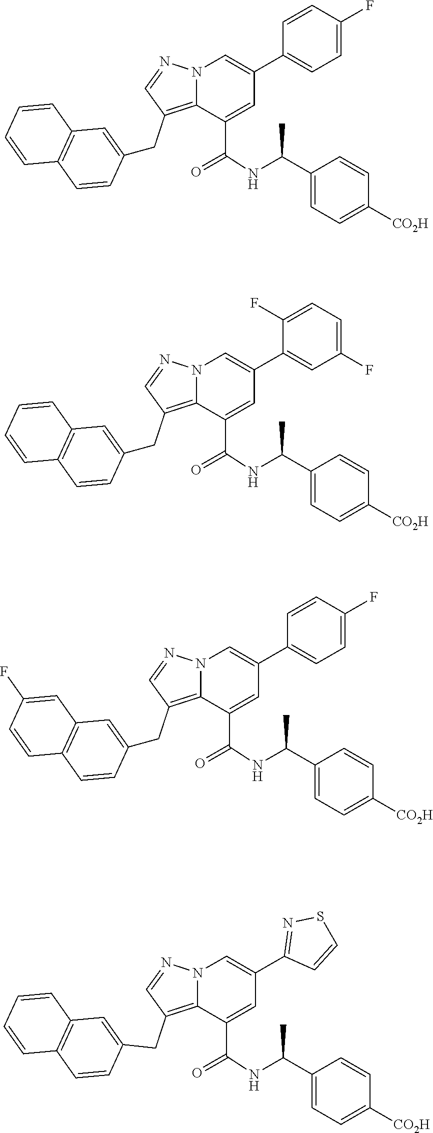 [SMILES: C[C@H](NC(=O)c1cc(-c2ccc(F)cc2)cn2ncc(Cc3ccc4ccccc4c3)c12)c1ccc(C(=O)O)cc1 ; C[C@H](NC(=O)c1cc(-c2cc(F)ccc2F)cn2ncc(Cc3ccc4ccccc4c3)c12)c1ccc(C(=O)O)cc1 ; C[C@H](NC(=O)c1cc(-c2ccc(F)cc2)cn2ncc(Cc3ccc4ccc(F)cc4c3)c12)c1ccc(C(=O)O)cc1 ; C[C@H](NC(=O)c1cc(-c2ccsn2)cn2ncc(Cc3ccc4ccccc4c3)c12)c1ccc(C(=O)O)cc1]

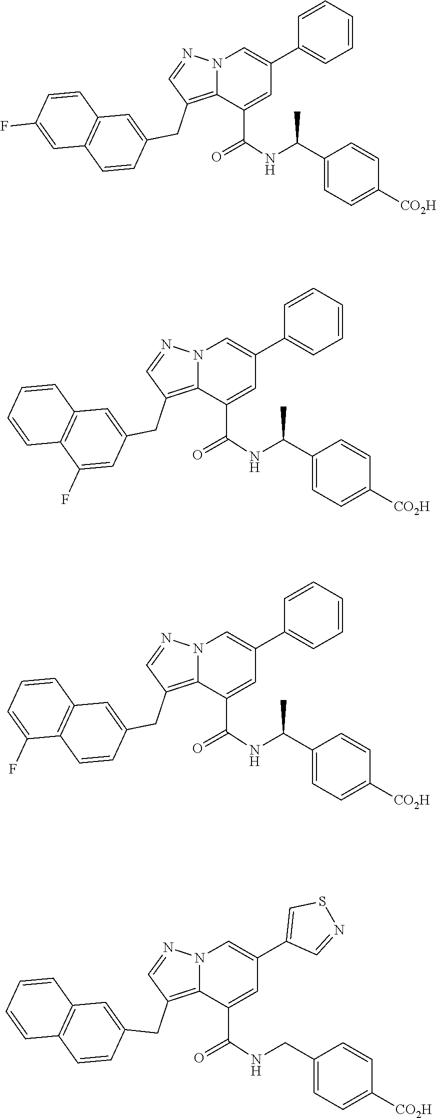 [SMILES: C[C@H](NC(=O)c1cc(-c2ccccc2)cn2ncc(Cc3ccc4cc(F)ccc4c3)c12)c1ccc(C(=O)O)cc1 ; C[C@H](NC(=O)c1cc(-c2ccccc2)cn2ncc(Cc3cc(F)c4ccccc4c3)c12)c1ccc(C(=O)O)cc1 ; C[C@H](NC(=O)c1cc(-c2ccccc2)cn2ncc(Cc3ccc4c(F)cccc4c3)c12)c1ccc(C(=O)O)cc1 ; O=C(O)c1ccc(CNC(=O)c2cc(-c3cnsc3)cn3ncc(Cc4ccc5ccccc5c4)c23)cc1]

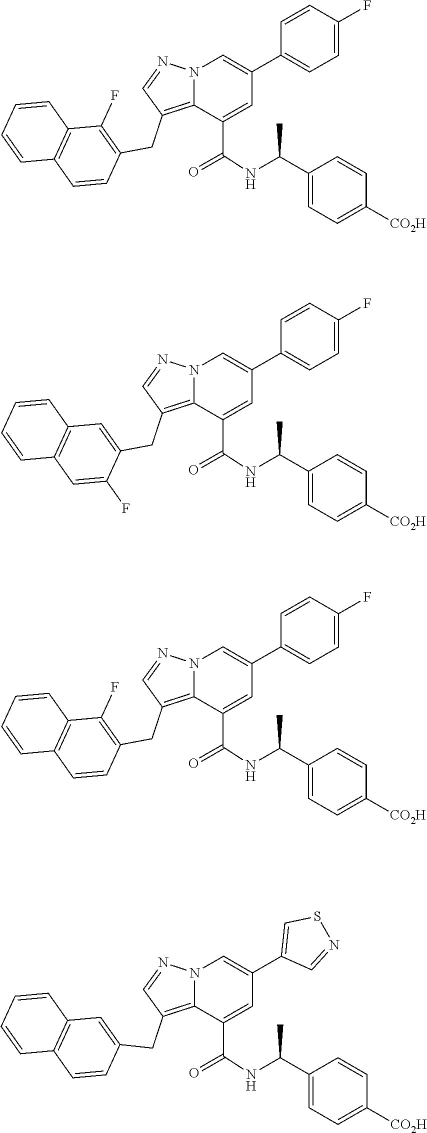 [SMILES: C[C@H](NC(=O)c1cc(-c2ccc(F)cc2)cn2ncc(Cc3ccc4ccccc4c3F)c12)c1ccc(C(=O)O)cc1 ; C[C@H](NC(=O)c1cc(-c2ccc(F)cc2)cn2ncc(Cc3cc4ccccc4cc3F)c12)c1ccc(C(=O)O)cc1 ; C[C@H](NC(=O)c1cc(-c2ccc(F)cc2)cn2ncc(Cc3ccc4ccccc4c3F)c12)c1ccc(C(=O)O)cc1 ; C[C@H](NC(=O)c1cc(-c2cnsc2)cn2ncc(Cc3ccc4ccccc4c3)c12)c1ccc(C(=O)O)cc1]

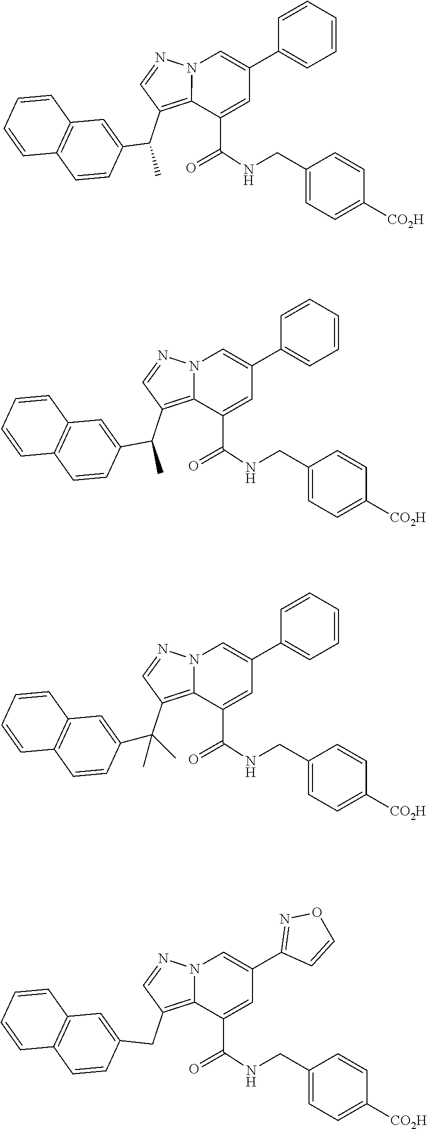 [SMILES: C[C@H](c1ccc2ccccc2c1)c1cnn2cc(-c3ccccc3)cc(C(=O)NCc3ccc(C(=O)O)cc3)c12 ; C[C@@H](c1ccc2ccccc2c1)c1cnn2cc(-c3ccccc3)cc(C(=O)NCc3ccc(C(=O)O)cc3)c12 ; CC(C)(c1ccc2ccccc2c1)c1cnn2cc(-c3ccccc3)cc(C(=O)NCc3ccc(C(=O)O)cc3)c12 ; O=C(O)c1ccc(CNC(=O)c2cc(-c3ccon3)cn3ncc(Cc4ccc5ccccc5c4)c23)cc1]

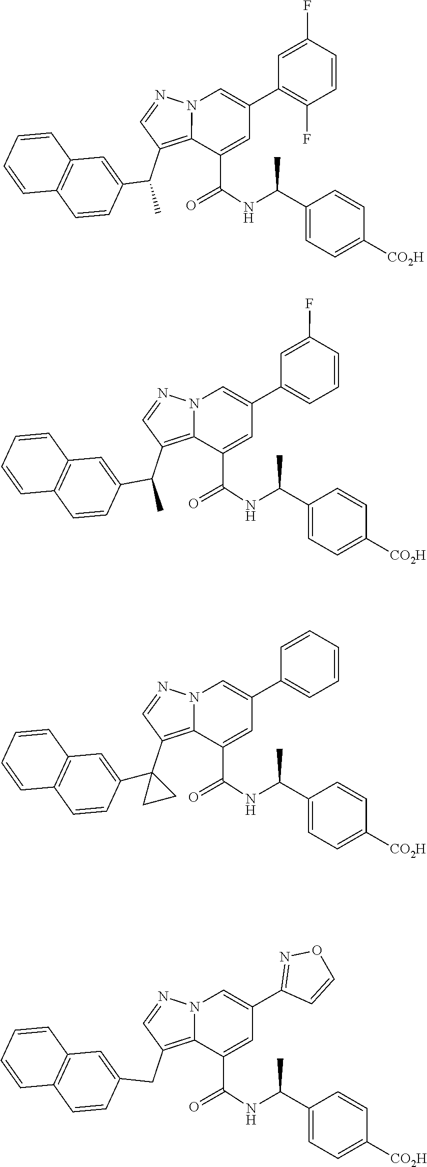 [SMILES: C[C@H](c1ccc2ccccc2c1)c1cnn2cc(-c3cc(F)ccc3F)cc(C(=O)N[C@@H](C)c3ccc(C(=O)O)cc3)c12 ; C[C@@H](c1ccc2ccccc2c1)c1cnn2cc(-c3cccc(F)c3)cc(C(=O)N[C@@H](C)c3ccc(C(=O)O)cc3)c12 ; C[C@H](NC(=O)c1cc(-c2ccccc2)cn2ncc(C3(c4ccc5ccccc5c4)CC3)c12)c1ccc(C(=O)O)cc1 ; C[C@H](NC(=O)c1cc(-c2ccon2)cn2ncc(Cc3ccc4ccccc4c3)c12)c1ccc(C(=O)O)cc1]

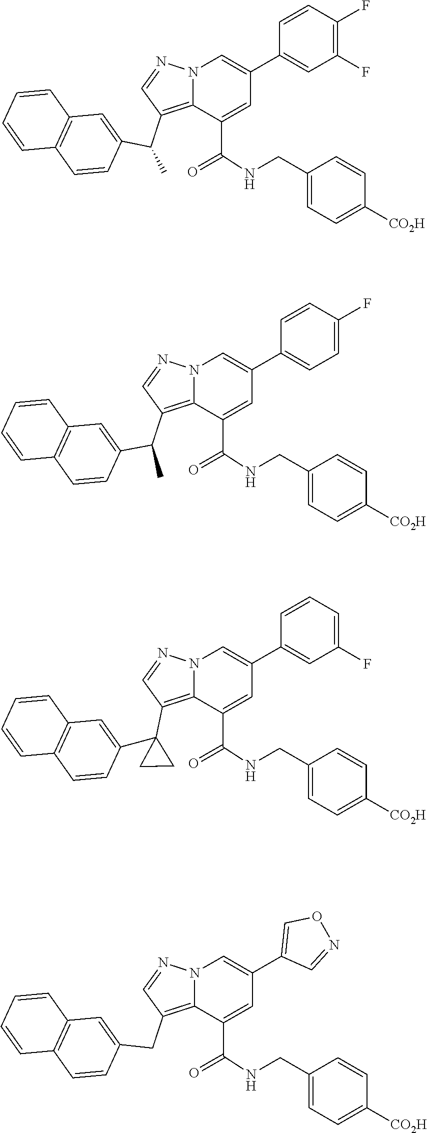 [SMILES: C[C@H](c1ccc2ccccc2c1)c1cnn2cc(-c3ccc(F)c(F)c3)cc(C(=O)NCc3ccc(C(=O)O)cc3)c12 ; C[C@@H](c1ccc2ccccc2c1)c1cnn2cc(-c3ccc(F)cc3)cc(C(=O)NCc3ccc(C(=O)O)cc3)c12 ; O=C(O)c1ccc(CNC(=O)c2cc(-c3cccc(F)c3)cn3ncc(C4(c5ccc6ccccc6c5)CC4)c23)cc1 ; O=C(O)c1ccc(CNC(=O)c2cc(-c3cnoc3)cn3ncc(Cc4ccc5ccccc5c4)c23)cc1]

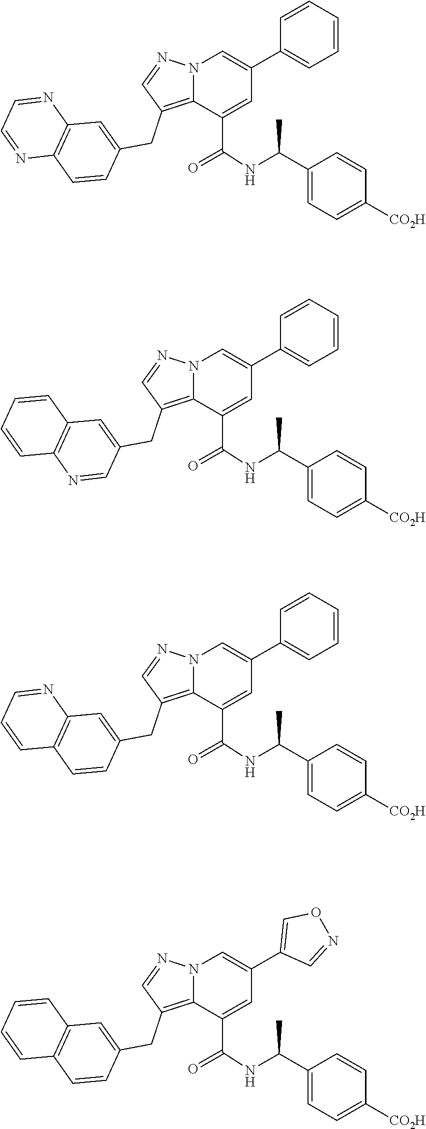 [SMILES: C[C@H](NC(=O)c1cc(-c2ccccc2)cn2ncc(Cc3ccc4nccnc4c3)c12)c1ccc(C(=O)O)cc1 ; C[C@H](NC(=O)c1cc(-c2ccccc2)cn2ncc(Cc3cnc4ccccc4c3)c12)c1ccc(C(=O)O)cc1 ; C[C@H](NC(=O)c1cc(-c2ccccc2)cn2ncc(Cc3ccc4cccnc4c3)c12)c1ccc(C(=O)O)cc1 ; C[C@H](NC(=O)c1cc(-c2cnoc2)cn2ncc(Cc3ccc4ccccc4c3)c12)c1ccc(C(=O)O)cc1]

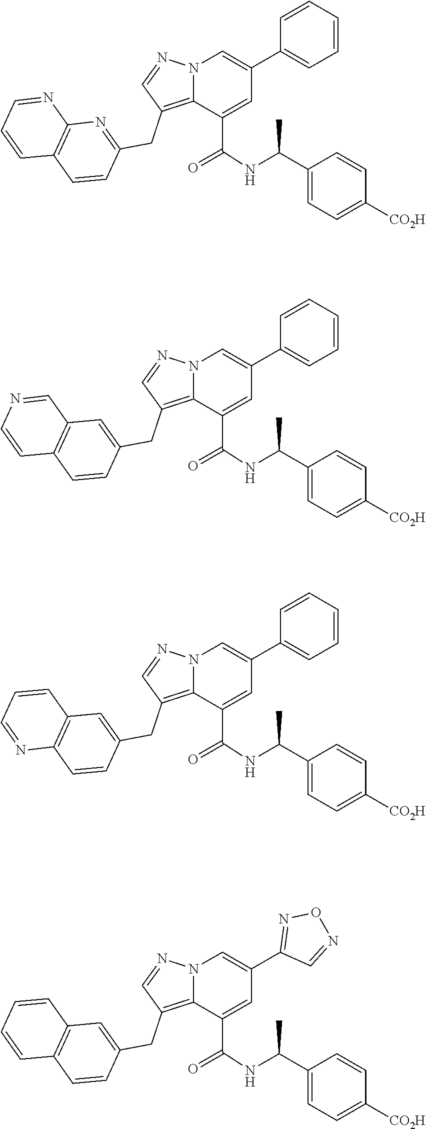 [SMILES: C[C@H](NC(=O)c1cc(-c2ccccc2)cn2ncc(Cc3ccc4cccnc4n3)c12)c1ccc(C(=O)O)cc1 ; C[C@H](NC(=O)c1cc(-c2ccccc2)cn2ncc(Cc3ccc4ccncc4c3)c12)c1ccc(C(=O)O)cc1 ; C[C@H](NC(=O)c1cc(-c2ccccc2)cn2ncc(Cc3ccc4ncccc4c3)c12)c1ccc(C(=O)O)cc1 ; C[C@H](NC(=O)c1cc(-c2cnon2)cn2ncc(Cc3ccc4ccccc4c3)c12)c1ccc(C(=O)O)cc1]

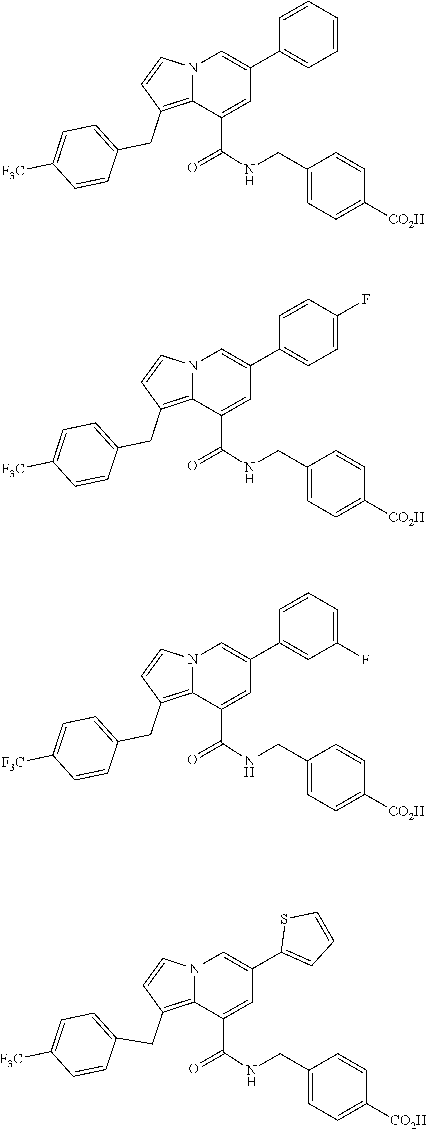 [SMILES: O=C(O)c1ccc(CNC(=O)c2cc(-c3ccccc3)cn3ccc(Cc4ccc(C(F)(F)F)cc4)c23)cc1 ; O=C(O)c1ccc(CNC(=O)c2cc(-c3ccc(F)cc3)cn3ccc(Cc4ccc(C(F)(F)F)cc4)c23)cc1 ; O=C(O)c1ccc(CNC(=O)c2cc(-c3cccc(F)c3)cn3ccc(Cc4ccc(C(F)(F)F)cc4)c23)cc1 ; O=C(O)c1ccc(CNC(=O)c2cc(-c3cccs3)cn3ccc(Cc4ccc(C(F)(F)F)cc4)c23)cc1]

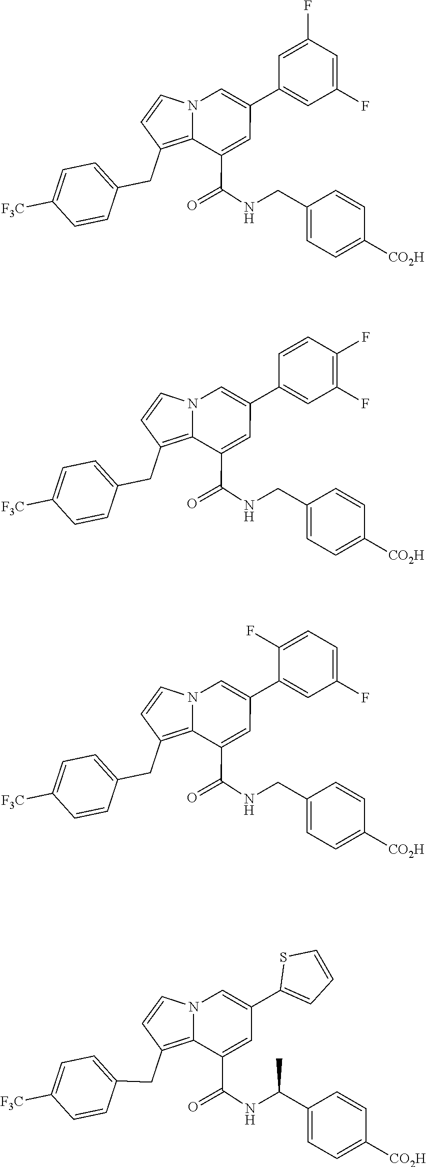 [SMILES: O=C(O)c1ccc(CNC(=O)c2cc(-c3cc(F)cc(F)c3)cn3ccc(Cc4ccc(C(F)(F)F)cc4)c23)cc1 ; O=C(O)c1ccc(CNC(=O)c2cc(-c3ccc(F)c(F)c3)cn3ccc(Cc4ccc(C(F)(F)F)cc4)c23)cc1 ; O=C(O)c1ccc(CNC(=O)c2cc(-c3cc(F)ccc3F)cn3ccc(Cc4ccc(C(F)(F)F)cc4)c23)cc1 ; C[C@H](NC(=O)c1cc(-c2cccs2)cn2ccc(Cc3ccc(C(F)(F)F)cc3)c12)c1ccc(C(=O)O)cc1]

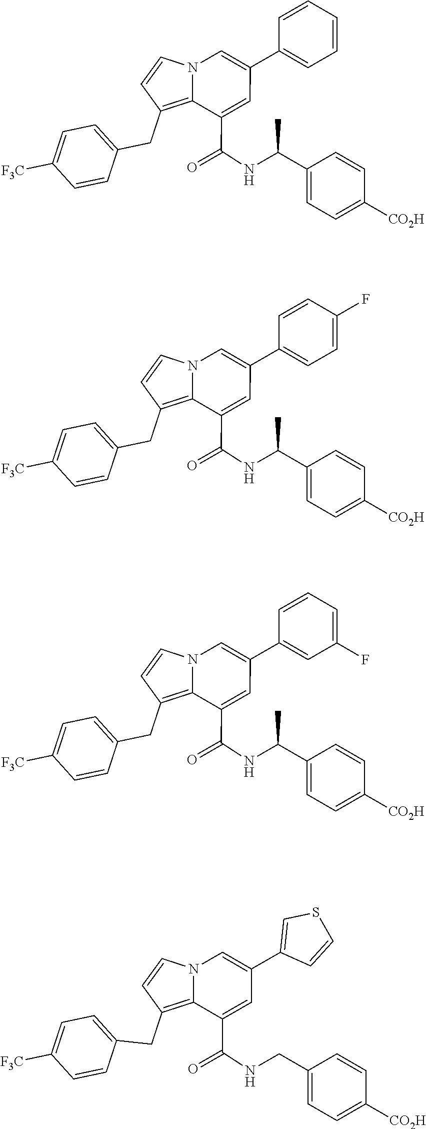 [SMILES: C[C@H](NC(=O)c1cc(-c2ccccc2)cn2ccc(Cc3ccc(C(F)(F)F)cc3)c12)c1ccc(C(=O)O)cc1 ; C[C@H](NC(=O)c1cc(-c2ccc(F)cc2)cn2ccc(Cc3ccc(C(F)(F)F)cc3)c12)c1ccc(C(=O)O)cc1 ; C[C@H](NC(=O)c1cc(-c2cccc(F)c2)cn2ccc(Cc3ccc(C(F)(F)F)cc3)c12)c1ccc(C(=O)O)cc1 ; O=C(O)c1ccc(CNC(=O)c2cc(-c3ccsc3)cn3ccc(Cc4ccc(C(F)(F)F)cc4)c23)cc1]

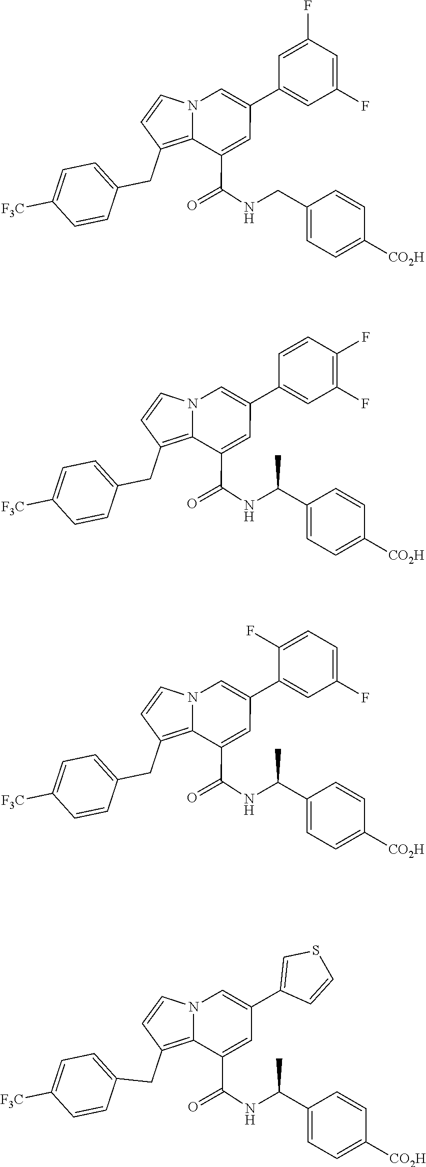 [SMILES: O=C(O)c1ccc(CNC(=O)c2cc(-c3cc(F)cc(F)c3)cn3ccc(Cc4ccc(C(F)(F)F)cc4)c23)cc1 ; C[C@H](NC(=O)c1cc(-c2ccc(F)c(F)c2)cn2ccc(Cc3ccc(C(F)(F)F)cc3)c12)c1ccc(C(=O)O)cc1 ; C[C@H](NC(=O)c1cc(-c2cc(F)ccc2F)cn2ccc(Cc3ccc(C(F)(F)F)cc3)c12)c1ccc(C(=O)O)cc1 ; C[C@H](NC(=O)c1cc(-c2ccsc2)cn2ccc(Cc3ccc(C(F)(F)F)cc3)c12)c1ccc(C(=O)O)cc1]

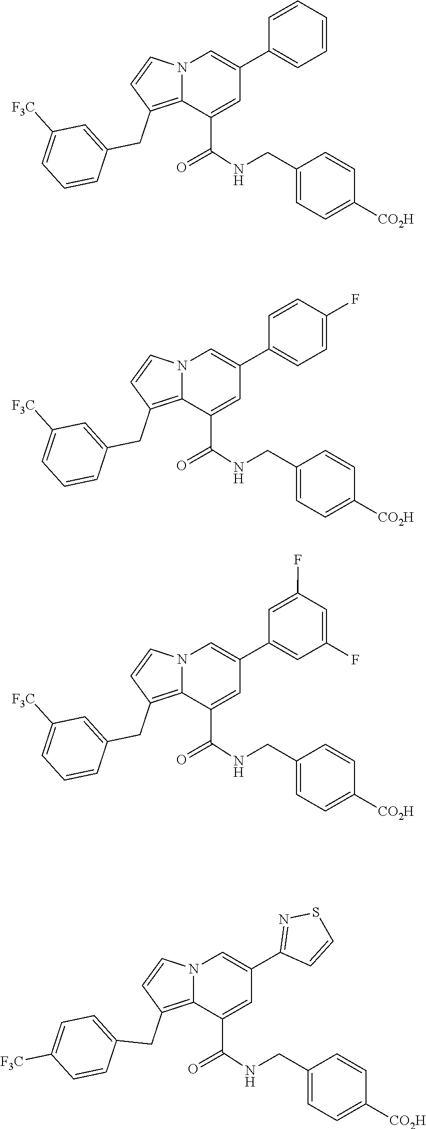 [SMILES: O=C(O)c1ccc(CNC(=O)c2cc(-c3ccccc3)cn3ccc(Cc4cccc(C(F)(F)F)c4)c23)cc1 ; O=C(O)c1ccc(CNC(=O)c2cc(-c3ccc(F)cc3)cn3ccc(Cc4cccc(C(F)(F)F)c4)c23)cc1 ; O=C(O)c1ccc(CNC(=O)c2cc(-c3cc(F)cc(F)c3)cn3ccc(Cc4cccc(C(F)(F)F)c4)c23)cc1 ; O=C(O)c1ccc(CNC(=O)c2cc(-c3ccsn3)cn3ccc(Cc4ccc(C(F)(F)F)cc4)c23)cc1]

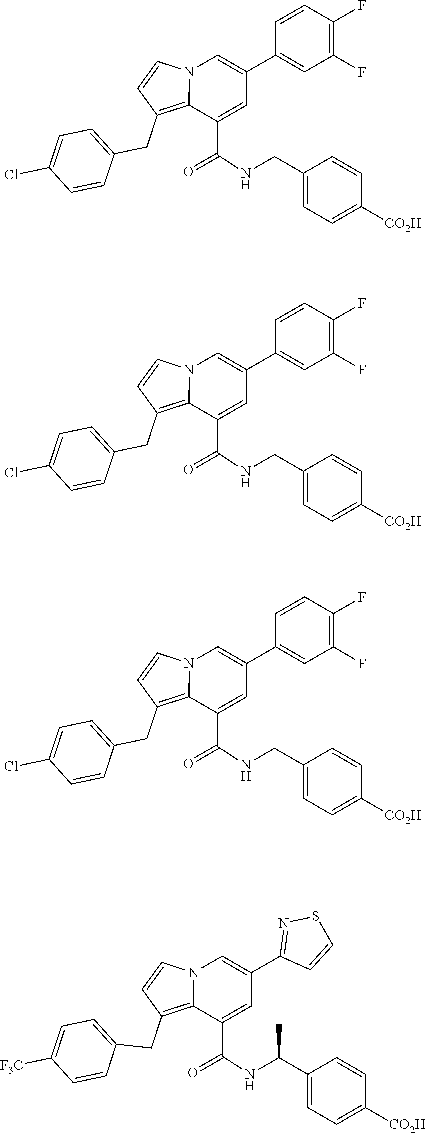 [SMILES: O=C(O)c1ccc(CNC(=O)c2cc(-c3ccc(F)c(F)c3)cn3ccc(Cc4ccc(Cl)cc4)c23)cc1 ; O=C(O)c1ccc(CNC(=O)c2cc(-c3ccc(F)c(F)c3)cn3ccc(Cc4ccc(Cl)cc4)c23)cc1 ; O=C(O)c1ccc(CNC(=O)c2cc(-c3ccc(F)c(F)c3)cn3ccc(Cc4ccc(Cl)cc4)c23)cc1 ; C[C@H](NC(=O)c1cc(-c2ccsn2)cn2ccc(Cc3ccc(C(F)(F)F)cc3)c12)c1ccc(C(=O)O)cc1]

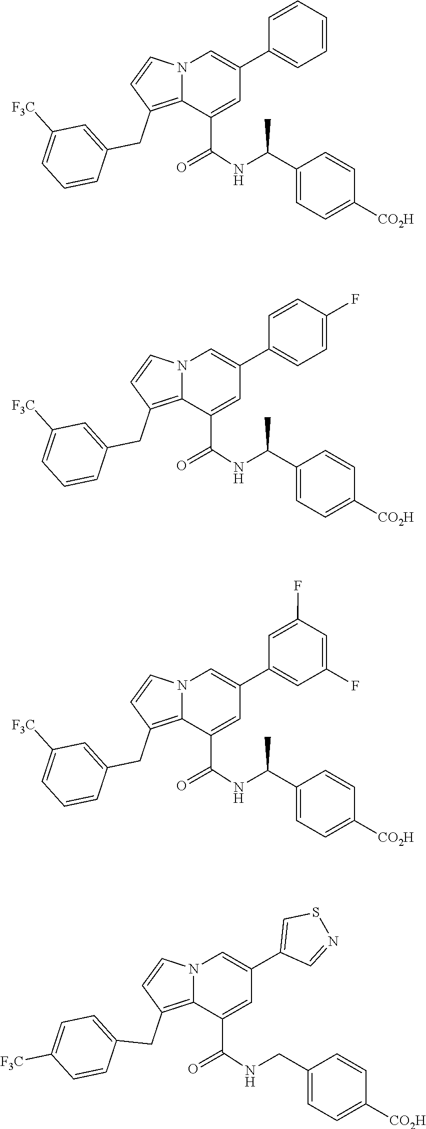 [SMILES: C[C@H](NC(=O)c1cc(-c2ccccc2)cn2ccc(Cc3cccc(C(F)(F)F)c3)c12)c1ccc(C(=O)O)cc1 ; C[C@H](NC(=O)c1cc(-c2ccc(F)cc2)cn2ccc(Cc3cccc(C(F)(F)F)c3)c12)c1ccc(C(=O)O)cc1 ; C[C@H](NC(=O)c1cc(-c2cc(F)cc(F)c2)cn2ccc(Cc3cccc(C(F)(F)F)c3)c12)c1ccc(C(=O)O)cc1 ; O=C(O)c1ccc(CNC(=O)c2cc(-c3cnsc3)cn3ccc(Cc4ccc(C(F)(F)F)cc4)c23)cc1]

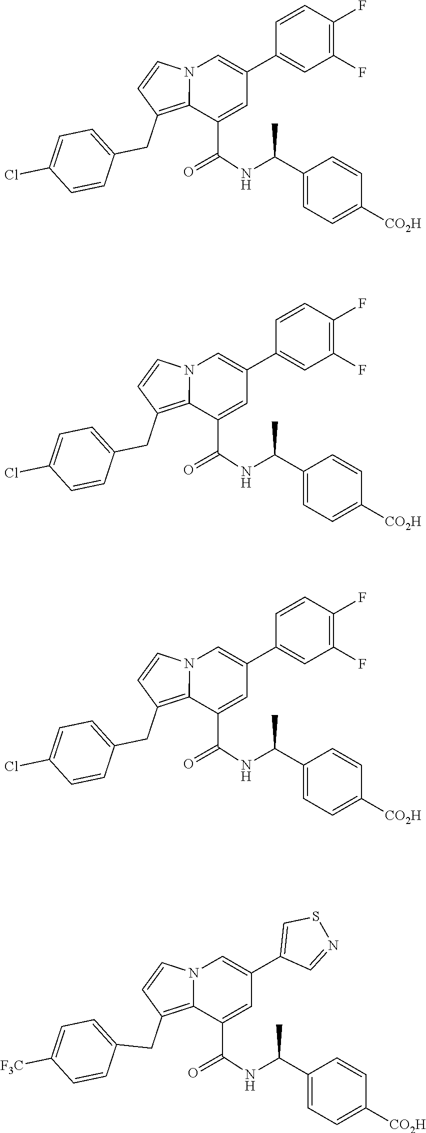 [SMILES: C[C@H](NC(=O)c1cc(-c2ccc(F)c(F)c2)cn2ccc(Cc3ccc(Cl)cc3)c12)c1ccc(C(=O)O)cc1 ; C[C@H](NC(=O)c1cc(-c2ccc(F)c(F)c2)cn2ccc(Cc3ccc(Cl)cc3)c12)c1ccc(C(=O)O)cc1 ; C[C@H](NC(=O)c1cc(-c2ccc(F)c(F)c2)cn2ccc(Cc3ccc(Cl)cc3)c12)c1ccc(C(=O)O)cc1 ; C[C@H](NC(=O)c1cc(-c2cnsc2)cn2ccc(Cc3ccc(C(F)(F)F)cc3)c12)c1ccc(C(=O)O)cc1]

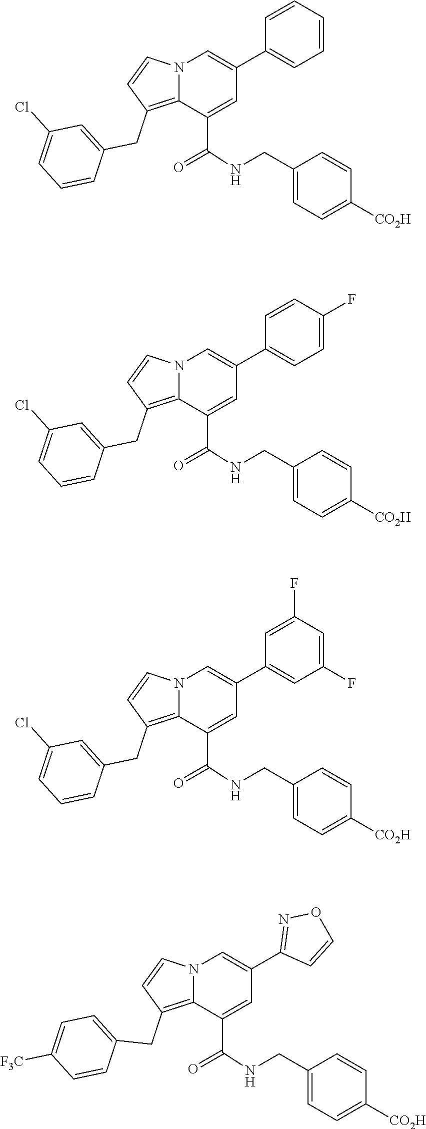 [SMILES: O=C(O)c1ccc(CNC(=O)c2cc(-c3ccccc3)cn3ccc(Cc4cccc(Cl)c4)c23)cc1 ; O=C(O)c1ccc(CNC(=O)c2cc(-c3ccc(F)cc3)cn3ccc(Cc4cccc(Cl)c4)c23)cc1 ; O=C(O)c1ccc(CNC(=O)c2cc(-c3cc(F)cc(F)c3)cn3ccc(Cc4cccc(Cl)c4)c23)cc1 ; O=C(O)c1ccc(CNC(=O)c2cc(-c3ccon3)cn3ccc(Cc4ccc(C(F)(F)F)cc4)c23)cc1]

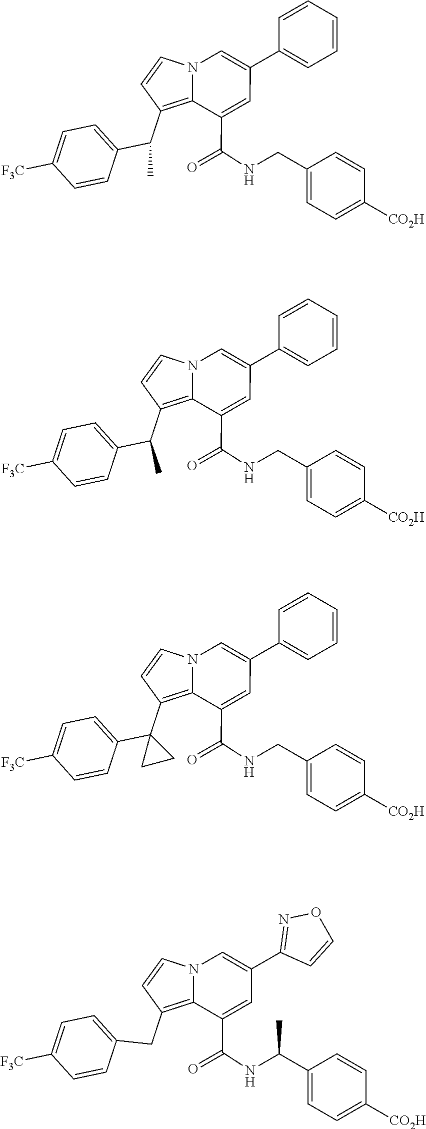 [SMILES: C[C@H](c1ccc(C(F)(F)F)cc1)c1ccn2cc(-c3ccccc3)cc(C(=O)NCc3ccc(C(=O)O)cc3)c12 ; C[C@@H](c1ccc(C(F)(F)F)cc1)c1ccn2cc(-c3ccccc3)cc(C(=O)NCc3ccc(C(=O)O)cc3)c12 ; O=C(O)c1ccc(CNC(=O)c2cc(-c3ccccc3)cn3ccc(C4(c5ccc(C(F)(F)F)cc5)CC4)c23)cc1 ; C[C@H](NC(=O)c1cc(-c2ccon2)cn2ccc(Cc3ccc(C(F)(F)F)cc3)c12)c1ccc(C(=O)O)cc1]

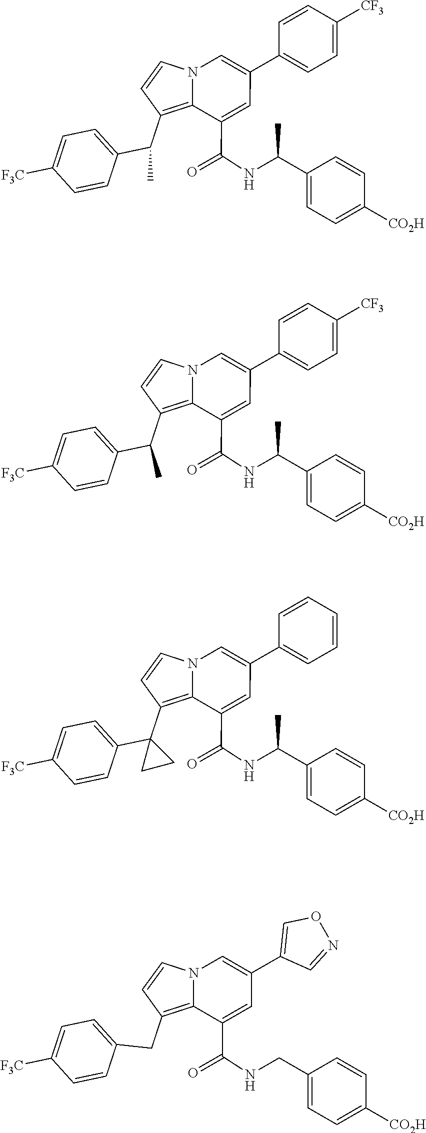 [SMILES: C[C@H](c1ccc(C(F)(F)F)cc1)c1ccn2cc(-c3ccc(C(F)(F)F)cc3)cc(C(=O)N[C@@H](C)c3ccc(C(=O)O)cc3)c12 ; C[C@@H](c1ccc(C(F)(F)F)cc1)c1ccn2cc(-c3ccc(C(F)(F)F)cc3)cc(C(=O)N[C@@H](C)c3ccc(C(=O)O)cc3)c12 ; C[C@H](NC(=O)c1cc(-c2ccccc2)cn2ccc(C3(c4ccc(C(F)(F)F)cc4)CC3)c12)c1ccc(C(=O)O)cc1 ; O=C(O)c1ccc(CNC(=O)c2cc(-c3cnoc3)cn3ccc(Cc4ccc(C(F)(F)F)cc4)c23)cc1]

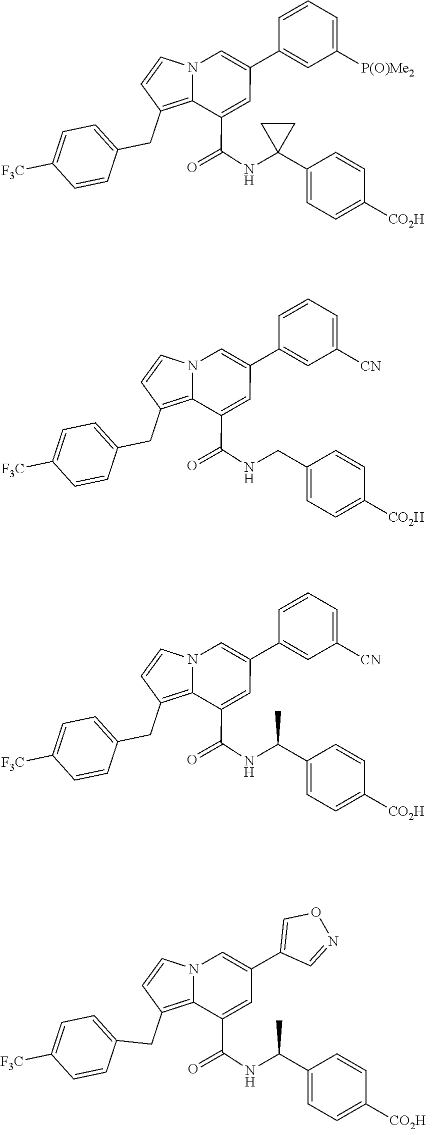

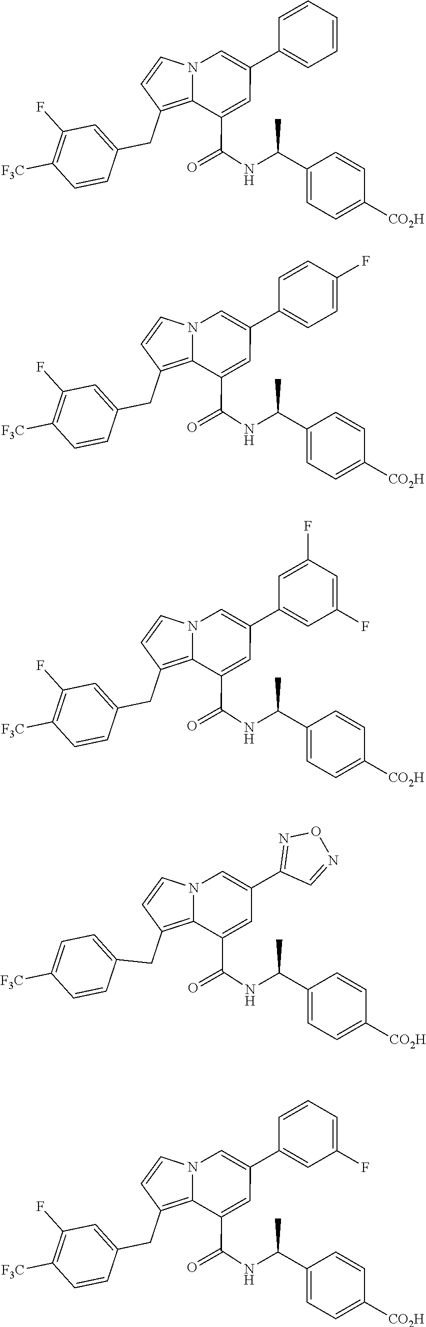 [SMILES: C[C@H](NC(=O)c1cc(-c2ccccc2)cn2ccc(Cc3ccc(C(F)(F)F)c(F)c3)c12)c1ccc(C(=O)O)cc1 ; C[C@H](NC(=O)c1cc(-c2ccc(F)cc2)cn2ccc(Cc3ccc(C(F)(F)F)c(F)c3)c12)c1ccc(C(=O)O)cc1 ; C[C@H](NC(=O)c1cc(-c2cc(F)cc(F)c2)cn2ccc(Cc3ccc(C(F)(F)F)c(F)c3)c12)c1ccc(C(=O)O)cc1 ; C[C@H](NC(=O)c1cc(-c2cnon2)cn2ccc(Cc3ccc(C(F)(F)F)cc3)c12)c1ccc(C(=O)O)cc1 ; C[C@H](NC(=O)c1cc(-c2cccc(F)c2)cn2ccc(Cc3ccc(C(F)(F)F)c(F)c3)c12)c1ccc(C(=O)O)cc1]

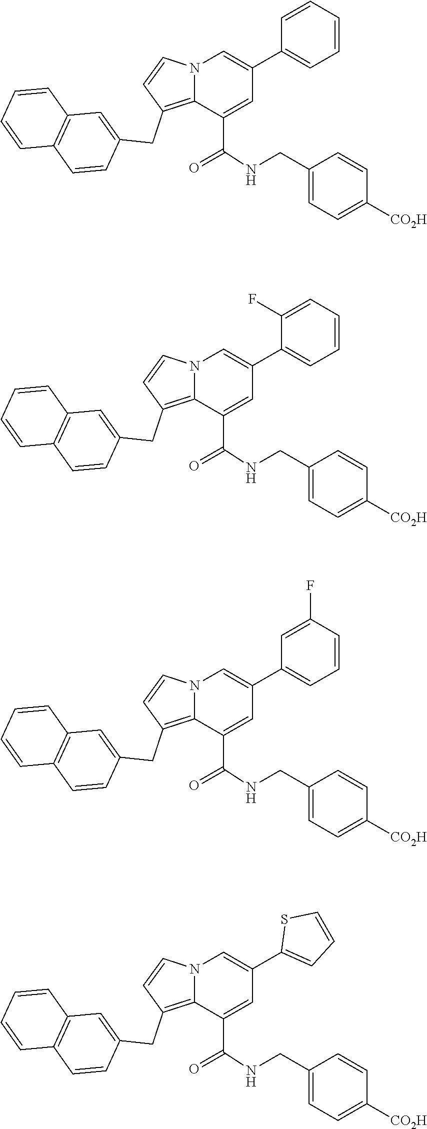 [SMILES: O=C(O)c1ccc(CNC(=O)c2cc(-c3ccccc3)cn3ccc(Cc4ccc5ccccc5c4)c23)cc1 ; O=C(O)c1ccc(CNC(=O)c2cc(-c3ccccc3F)cn3ccc(Cc4ccc5ccccc5c4)c23)cc1 ; O=C(O)c1ccc(CNC(=O)c2cc(-c3cccc(F)c3)cn3ccc(Cc4ccc5ccccc5c4)c23)cc1 ; O=C(O)c1ccc(CNC(=O)c2cc(-c3cccs3)cn3ccc(Cc4ccc5ccccc5c4)c23)cc1]

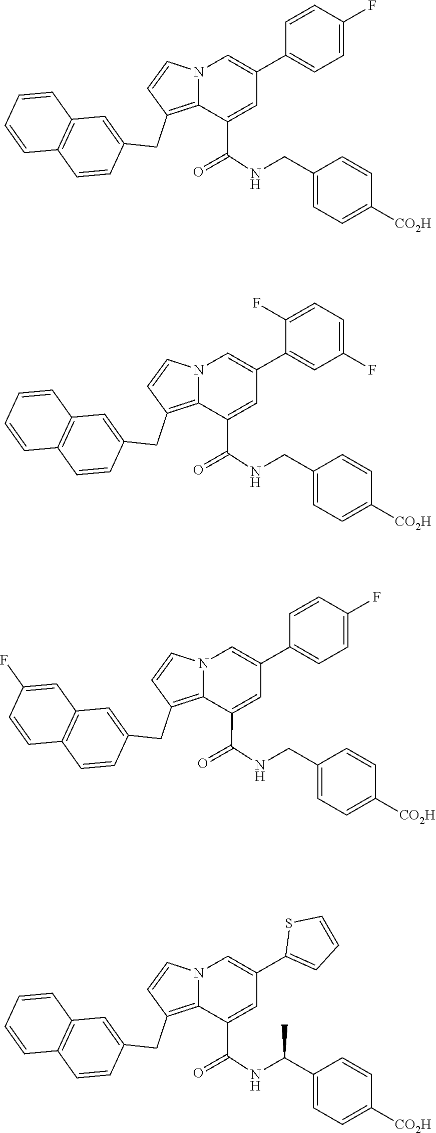 [SMILES: O=C(O)c1ccc(CNC(=O)c2cc(-c3ccc(F)cc3)cn3ccc(Cc4ccc5ccccc5c4)c23)cc1 ; O=C(O)c1ccc(CNC(=O)c2cc(-c3cc(F)ccc3F)cn3ccc(Cc4ccc5ccccc5c4)c23)cc1 ; O=C(O)c1ccc(CNC(=O)c2cc(-c3ccc(F)cc3)cn3ccc(Cc4ccc5ccc(F)cc5c4)c23)cc1 ; C[C@H](NC(=O)c1cc(-c2cccs2)cn2ccc(Cc3ccc4ccccc4c3)c12)c1ccc(C(=O)O)cc1]

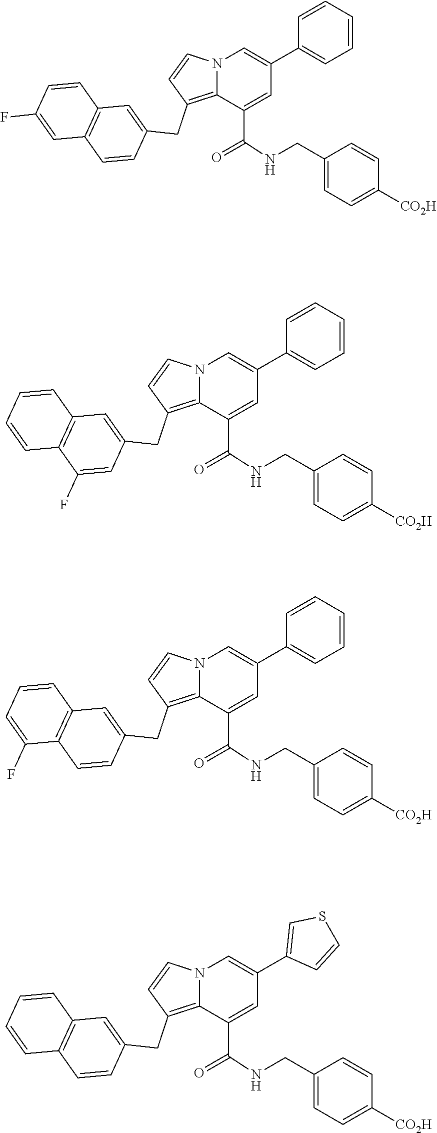 [SMILES: O=C(O)c1ccc(CNC(=O)c2cc(-c3ccccc3)cn3ccc(Cc4ccc5cc(F)ccc5c4)c23)cc1 ; O=C(O)c1ccc(CNC(=O)c2cc(-c3ccccc3)cn3ccc(Cc4cc(F)c5ccccc5c4)c23)cc1 ; O=C(O)c1ccc(CNC(=O)c2cc(-c3ccccc3)cn3ccc(Cc4ccc5c(F)cccc5c4)c23)cc1 ; O=C(O)c1ccc(CNC(=O)c2cc(-c3ccsc3)cn3ccc(Cc4ccc5ccccc5c4)c23)cc1]

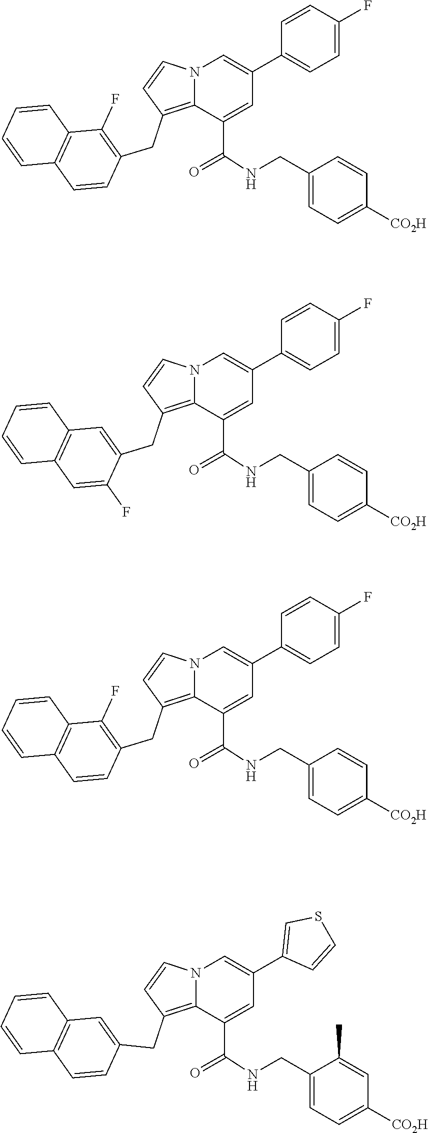

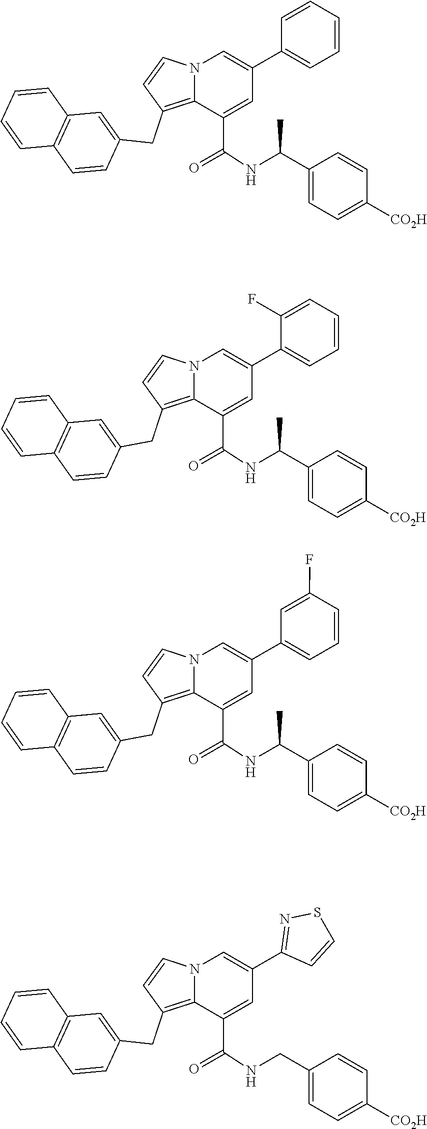 [SMILES: C[C@H](NC(=O)c1cc(-c2ccccc2)cn2ccc(Cc3ccc4ccccc4c3)c12)c1ccc(C(=O)O)cc1 ; C[C@H](NC(=O)c1cc(-c2ccccc2F)cn2ccc(Cc3ccc4ccccc4c3)c12)c1ccc(C(=O)O)cc1 ; C[C@H](NC(=O)c1cc(-c2cccc(F)c2)cn2ccc(Cc3ccc4ccccc4c3)c12)c1ccc(C(=O)O)cc1 ; O=C(O)c1ccc(CNC(=O)c2cc(-c3ccsn3)cn3ccc(Cc4ccc5ccccc5c4)c23)cc1]

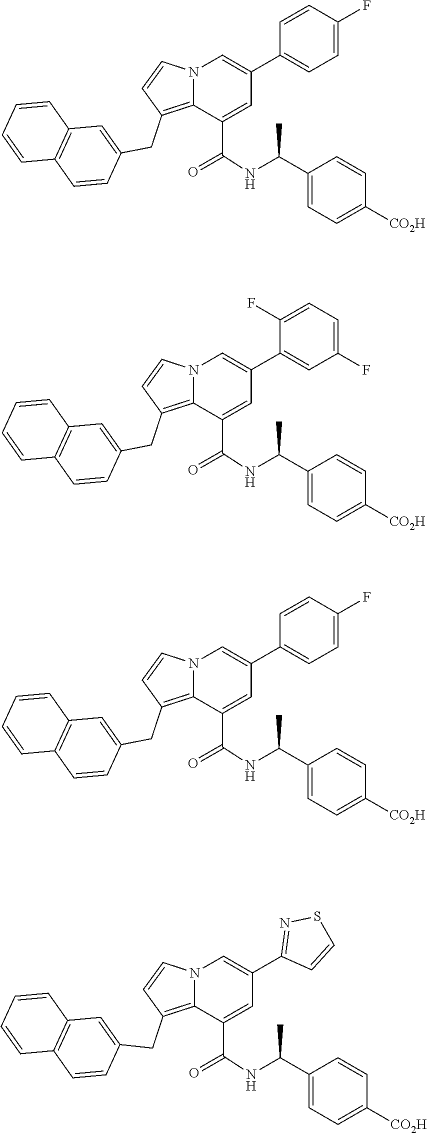 [SMILES: C[C@H](NC(=O)c1cc(-c2ccc(F)cc2)cn2ccc(Cc3ccc4ccccc4c3)c12)c1ccc(C(=O)O)cc1 ; C[C@H](NC(=O)c1cc(-c2cc(F)ccc2F)cn2ccc(Cc3ccc4ccccc4c3)c12)c1ccc(C(=O)O)cc1 ; C[C@H](NC(=O)c1cc(-c2ccc(F)cc2)cn2ccc(Cc3ccc4ccccc4c3)c12)c1ccc(C(=O)O)cc1 ; C[C@H](NC(=O)c1cc(-c2ccsn2)cn2ccc(Cc3ccc4ccccc4c3)c12)c1ccc(C(=O)O)cc1]

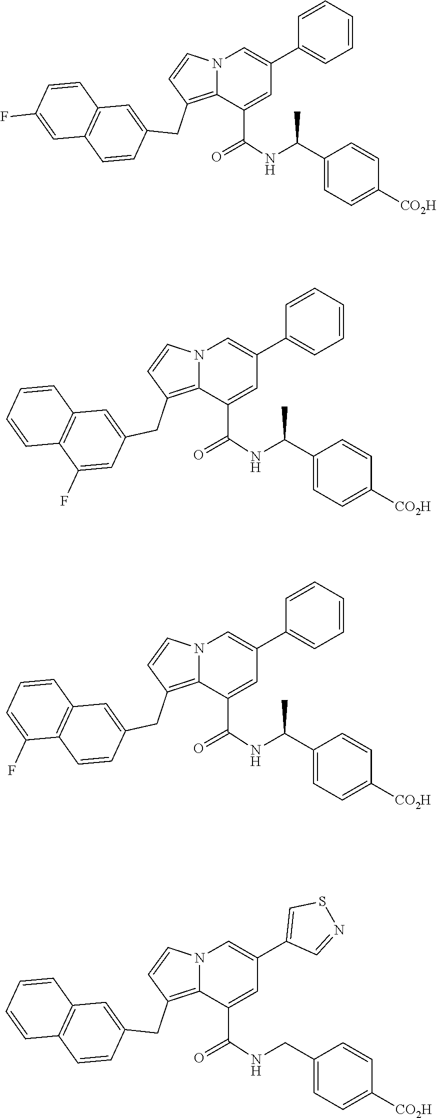 [SMILES: C[C@H](NC(=O)c1cc(-c2ccccc2)cn2ccc(Cc3ccc4cc(F)ccc4c3)c12)c1ccc(C(=O)O)cc1 ; C[C@H](NC(=O)c1cc(-c2ccccc2)cn2ccc(Cc3cc(F)c4ccccc4c3)c12)c1ccc(C(=O)O)cc1 ; C[C@H](NC(=O)c1cc(-c2ccccc2)cn2ccc(Cc3ccc4c(F)cccc4c3)c12)c1ccc(C(=O)O)cc1 ; O=C(O)c1ccc(CNC(=O)c2cc(-c3cnsc3)cn3ccc(Cc4ccc5ccccc5c4)c23)cc1]

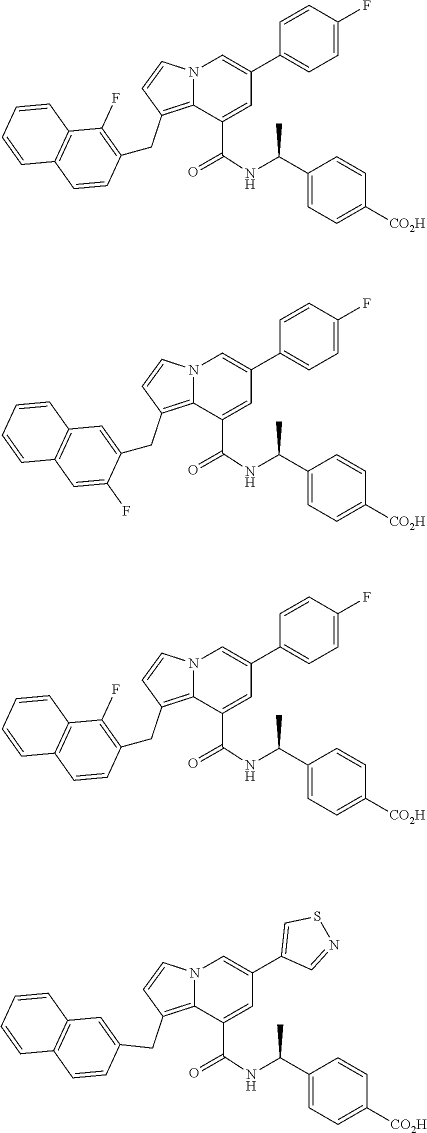 [SMILES: C[C@H](NC(=O)c1cc(-c2ccc(F)cc2)cn2ccc(Cc3ccc4ccccc4c3F)c12)c1ccc(C(=O)O)cc1 ; C[C@H](NC(=O)c1cc(-c2ccc(F)cc2)cn2ccc(Cc3cc4ccccc4cc3F)c12)c1ccc(C(=O)O)cc1 ; C[C@H](NC(=O)c1cc(-c2ccc(F)cc2)cn2ccc(Cc3ccc4ccccc4c3F)c12)c1ccc(C(=O)O)cc1 ; C[C@H](NC(=O)c1cc(-c2cnsc2)cn2ccc(Cc3ccc4ccccc4c3)c12)c1ccc(C(=O)O)cc1]

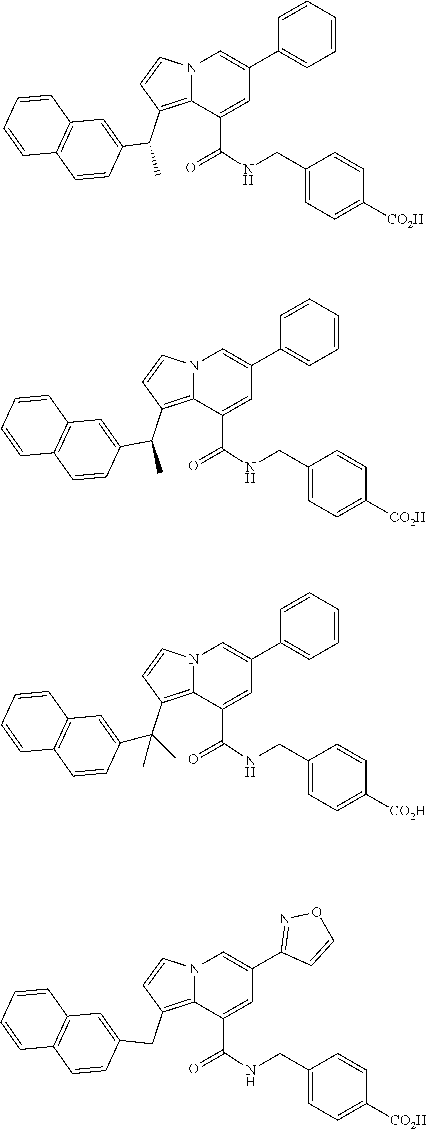 [SMILES: C[C@H](c1ccc2ccccc2c1)c1ccn2cc(-c3ccccc3)cc(C(=O)NCc3ccc(C(=O)O)cc3)c12 ; C[C@@H](c1ccc2ccccc2c1)c1ccn2cc(-c3ccccc3)cc(C(=O)NCc3ccc(C(=O)O)cc3)c12 ; CC(C)(c1ccc2ccccc2c1)c1ccn2cc(-c3ccccc3)cc(C(=O)NCc3ccc(C(=O)O)cc3)c12 ; O=C(O)c1ccc(CNC(=O)c2cc(-c3ccon3)cn3ccc(Cc4ccc5ccccc5c4)c23)cc1]

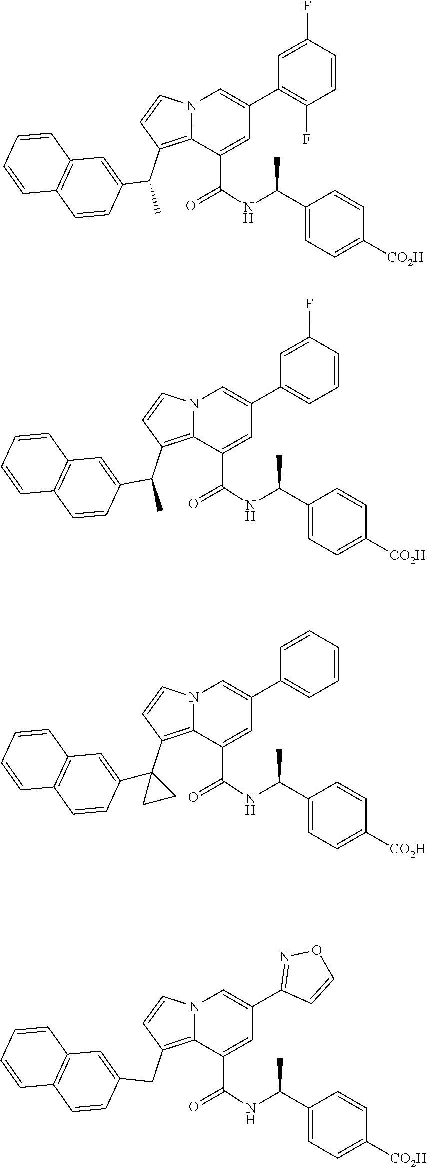 [SMILES: C[C@H](c1ccc2ccccc2c1)c1ccn2cc(-c3cc(F)ccc3F)cc(C(=O)N[C@@H](C)c3ccc(C(=O)O)cc3)c12 ; C[C@@H](c1ccc2ccccc2c1)c1ccn2cc(-c3cccc(F)c3)cc(C(=O)N[C@@H](C)c3ccc(C(=O)O)cc3)c12 ; C[C@H](NC(=O)c1cc(-c2ccccc2)cn2ccc(C3(c4ccc5ccccc5c4)CC3)c12)c1ccc(C(=O)O)cc1 ; C[C@H](NC(=O)c1cc(-c2ccon2)cn2ccc(Cc3ccc4ccccc4c3)c12)c1ccc(C(=O)O)cc1]

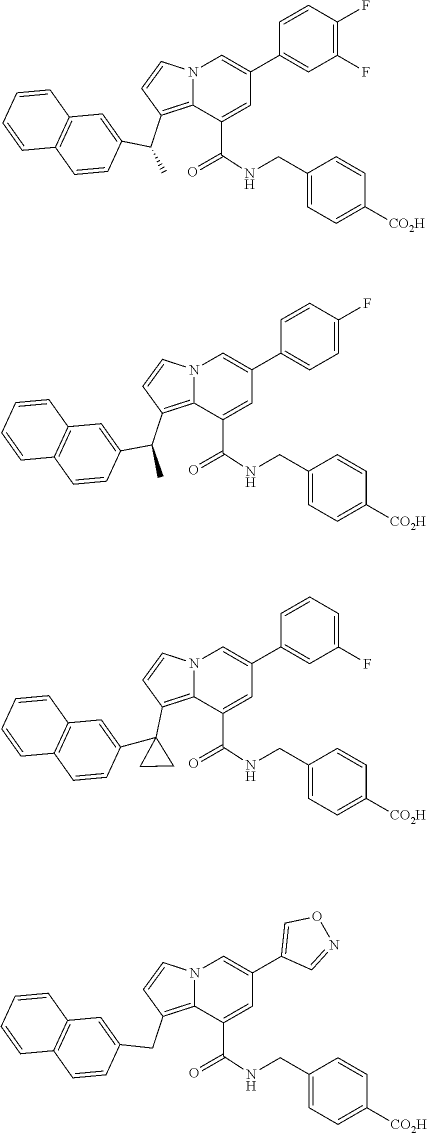 [SMILES: C[C@H](c1ccc2ccccc2c1)c1ccn2cc(-c3ccc(F)c(F)c3)cc(C(=O)NCc3ccc(C(=O)O)cc3)c12 ; C[C@@H](c1ccc2ccccc2c1)c1ccn2cc(-c3ccc(F)cc3)cc(C(=O)NCc3ccc(C(=O)O)cc3)c12 ; O=C(O)c1ccc(CNC(=O)c2cc(-c3cccc(F)c3)cn3ccc(C4(c5ccc6ccccc6c5)CC4)c23)cc1 ; O=C(O)c1ccc(CNC(=O)c2cc(-c3cnoc3)cn3ccc(Cc4ccc5ccccc5c4)c23)cc1]

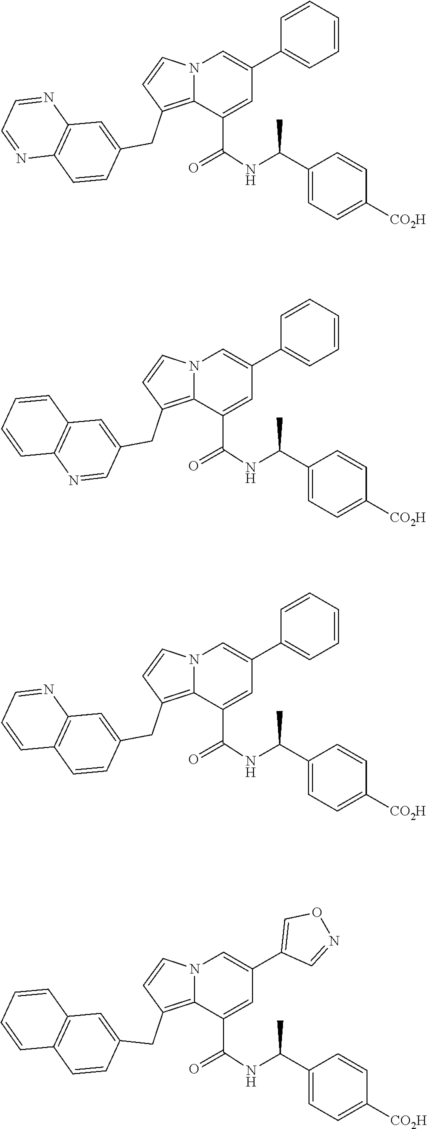 [SMILES: C[C@H](NC(=O)c1cc(-c2ccccc2)cn2ccc(Cc3ccc4nccnc4c3)c12)c1ccc(C(=O)O)cc1 ; C[C@H](NC(=O)c1cc(-c2ccccc2)cn2ccc(Cc3cnc4ccccc4c3)c12)c1ccc(C(=O)O)cc1 ; C[C@H](NC(=O)c1cc(-c2ccccc2)cn2ccc(Cc3ccc4cccnc4c3)c12)c1ccc(C(=O)O)cc1 ; C[C@H](NC(=O)c1cc(-c2cnoc2)cn2ccc(Cc3ccc4ccccc4c3)c12)c1ccc(C(=O)O)cc1]

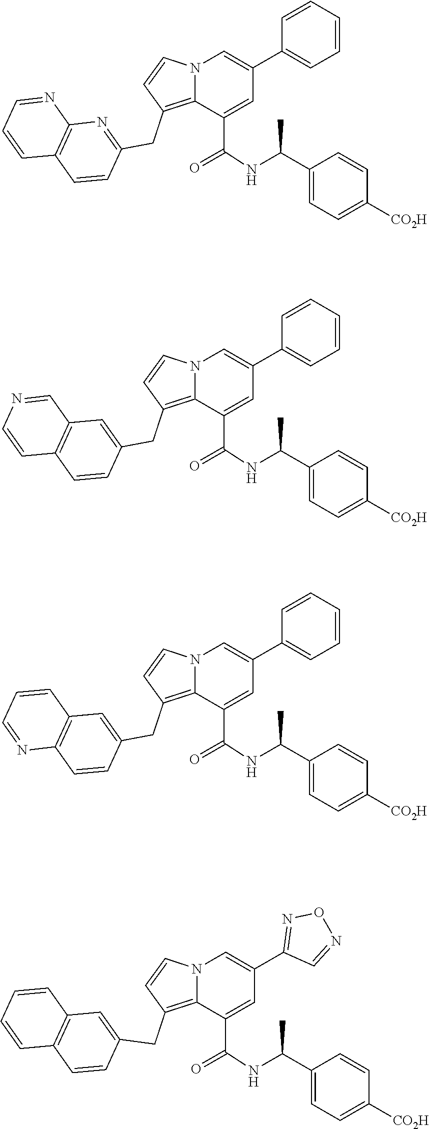 [SMILES: C[C@H](NC(=O)c1cc(-c2ccccc2)cn2ccc(Cc3ccc4cccnc4n3)c12)c1ccc(C(=O)O)cc1 ; C[C@H](NC(=O)c1cc(-c2ccccc2)cn2ccc(Cc3ccc4ccncc4c3)c12)c1ccc(C(=O)O)cc1 ; C[C@H](NC(=O)c1cc(-c2ccccc2)cn2ccc(Cc3ccc4ncccc4c3)c12)c1ccc(C(=O)O)cc1 ; C[C@H](NC(=O)c1cc(-c2cnon2)cn2ccc(Cc3ccc4ccccc4c3)c12)c1ccc(C(=O)O)cc1]

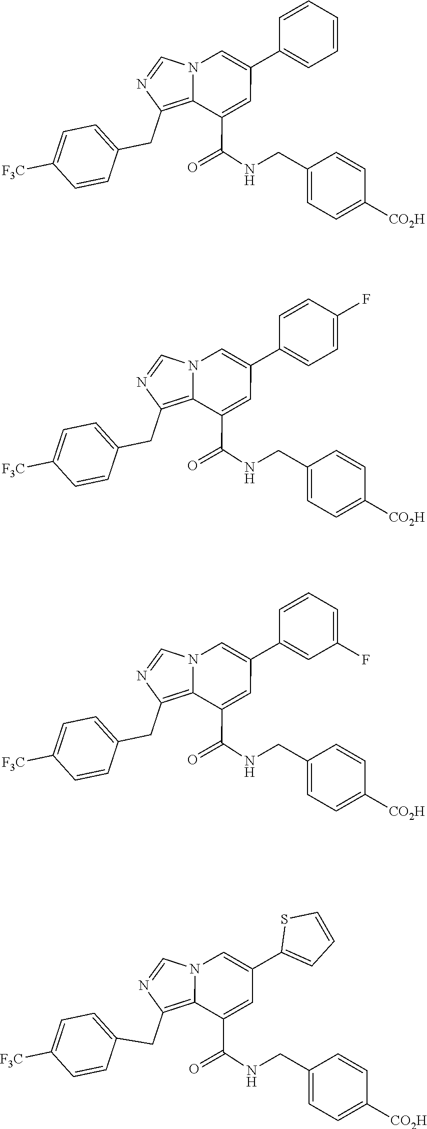 [SMILES: O=C(O)c1ccc(CNC(=O)c2cc(-c3ccccc3)cn3cnc(Cc4ccc(C(F)(F)F)cc4)c23)cc1 ; O=C(O)c1ccc(CNC(=O)c2cc(-c3ccc(F)cc3)cn3cnc(Cc4ccc(C(F)(F)F)cc4)c23)cc1 ; O=C(O)c1ccc(CNC(=O)c2cc(-c3cccc(F)c3)cn3cnc(Cc4ccc(C(F)(F)F)cc4)c23)cc1 ; O=C(O)c1ccc(CNC(=O)c2cc(-c3cccs3)cn3cnc(Cc4ccc(C(F)(F)F)cc4)c23)cc1]

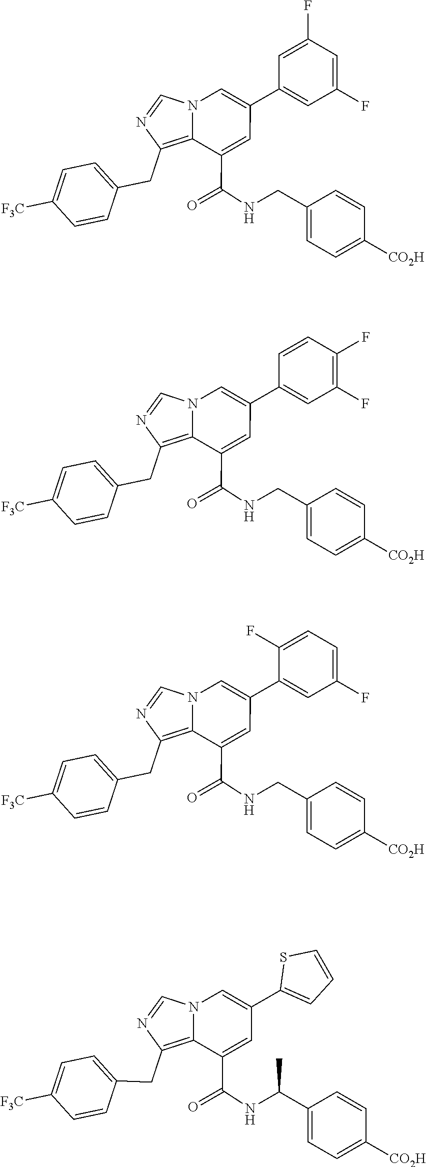 [SMILES: O=C(O)c1ccc(CNC(=O)c2cc(-c3cc(F)cc(F)c3)cn3cnc(Cc4ccc(C(F)(F)F)cc4)c23)cc1 ; O=C(O)c1ccc(CNC(=O)c2cc(-c3ccc(F)c(F)c3)cn3cnc(Cc4ccc(C(F)(F)F)cc4)c23)cc1 ; O=C(O)c1ccc(CNC(=O)c2cc(-c3cc(F)ccc3F)cn3cnc(Cc4ccc(C(F)(F)F)cc4)c23)cc1 ; C[C@H](NC(=O)c1cc(-c2cccs2)cn2cnc(Cc3ccc(C(F)(F)F)cc3)c12)c1ccc(C(=O)O)cc1]

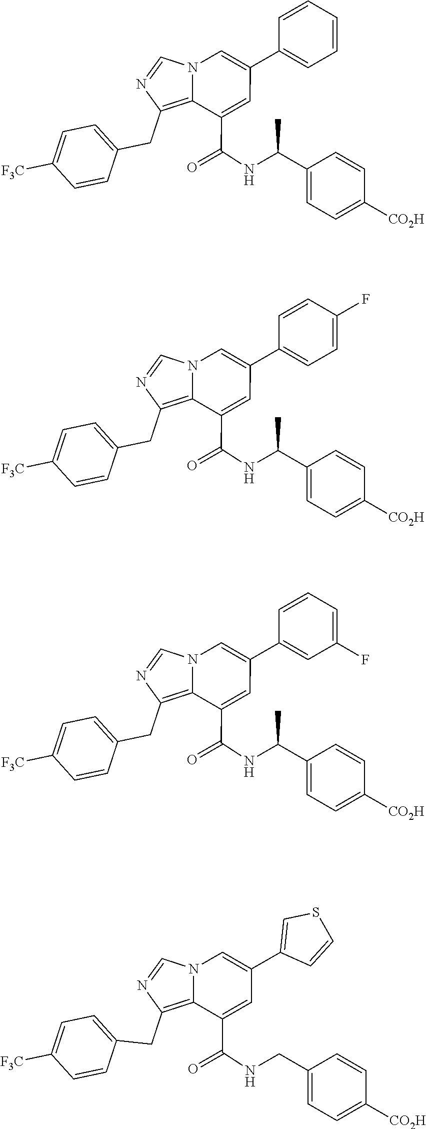 [SMILES: C[C@H](NC(=O)c1cc(-c2ccccc2)cn2cnc(Cc3ccc(C(F)(F)F)cc3)c12)c1ccc(C(=O)O)cc1 ; C[C@H](NC(=O)c1cc(-c2ccc(F)cc2)cn2cnc(Cc3ccc(C(F)(F)F)cc3)c12)c1ccc(C(=O)O)cc1 ; C[C@H](NC(=O)c1cc(-c2cccc(F)c2)cn2cnc(Cc3ccc(C(F)(F)F)cc3)c12)c1ccc(C(=O)O)cc1 ; O=C(O)c1ccc(CNC(=O)c2cc(-c3ccsc3)cn3cnc(Cc4ccc(C(F)(F)F)cc4)c23)cc1]

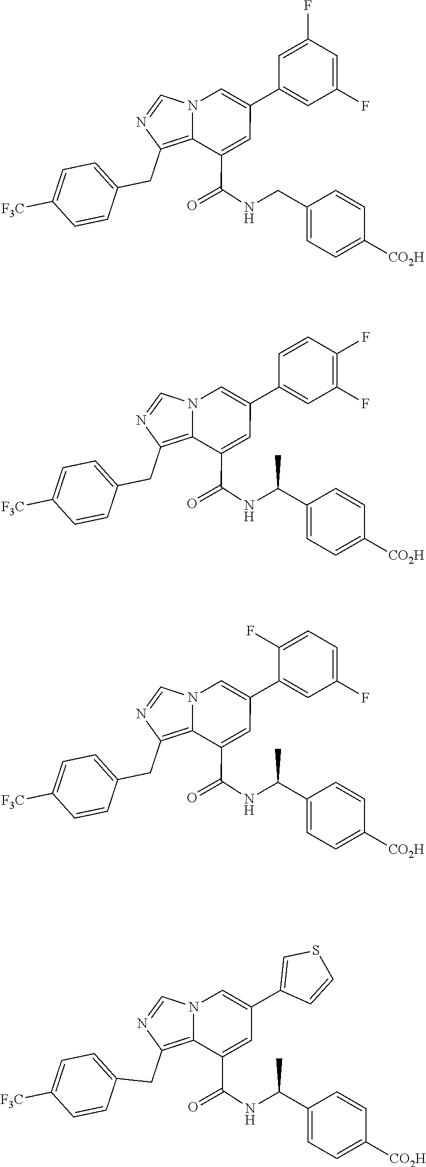 [SMILES: O=C(O)c1ccc(CNC(=O)c2cc(-c3cc(F)cc(F)c3)cn3cnc(Cc4ccc(C(F)(F)F)cc4)c23)cc1 ; C[C@H](NC(=O)c1cc(-c2ccc(F)c(F)c2)cn2cnc(Cc3ccc(C(F)(F)F)cc3)c12)c1ccc(C(=O)O)cc1 ; C[C@H](NC(=O)c1cc(-c2cc(F)ccc2F)cn2cnc(Cc3ccc(C(F)(F)F)cc3)c12)c1ccc(C(=O)O)cc1 ; C[C@H](NC(=O)c1cc(-c2ccsc2)cn2cnc(Cc3ccc(C(F)(F)F)cc3)c12)c1ccc(C(=O)O)cc1]

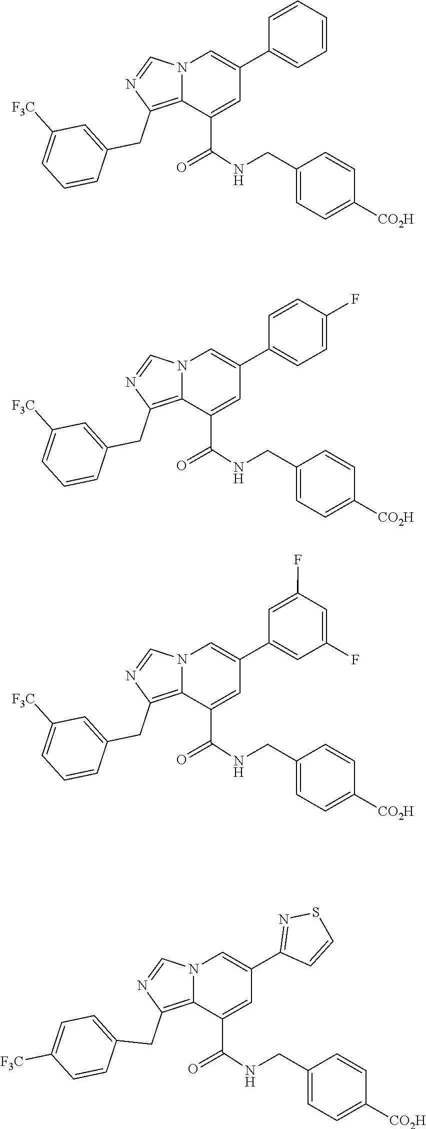 [SMILES: O=C(O)c1ccc(CNC(=O)c2cc(-c3ccccc3)cn3cnc(Cc4cccc(C(F)(F)F)c4)c23)cc1 ; O=C(O)c1ccc(CNC(=O)c2cc(-c3ccc(F)cc3)cn3cnc(Cc4cccc(C(F)(F)F)c4)c23)cc1 ; O=C(O)c1ccc(CNC(=O)c2cc(-c3cc(F)cc(F)c3)cn3cnc(Cc4cccc(C(F)(F)F)c4)c23)cc1 ; O=C(O)c1ccc(CNC(=O)c2cc(-c3ccsn3)cn3cnc(Cc4ccc(C(F)(F)F)cc4)c23)cc1]

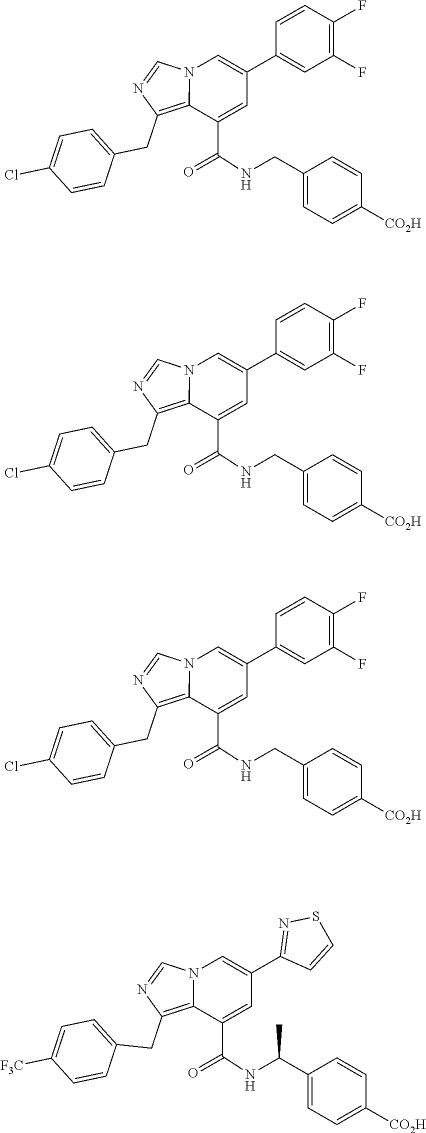 [SMILES: O=C(O)c1ccc(CNC(=O)c2cc(-c3ccc(F)c(F)c3)cn3cnc(Cc4ccc(Cl)cc4)c23)cc1 ; O=C(O)c1ccc(CNC(=O)c2cc(-c3ccc(F)c(F)c3)cn3cnc(Cc4ccc(Cl)cc4)c23)cc1 ; O=C(O)c1ccc(CNC(=O)c2cc(-c3ccc(F)c(F)c3)cn3cnc(Cc4ccc(Cl)cc4)c23)cc1 ; C[C@H](NC(=O)c1cc(-c2ccsn2)cn2cnc(Cc3ccc(C(F)(F)F)cc3)c12)c1ccc(C(=O)O)cc1]

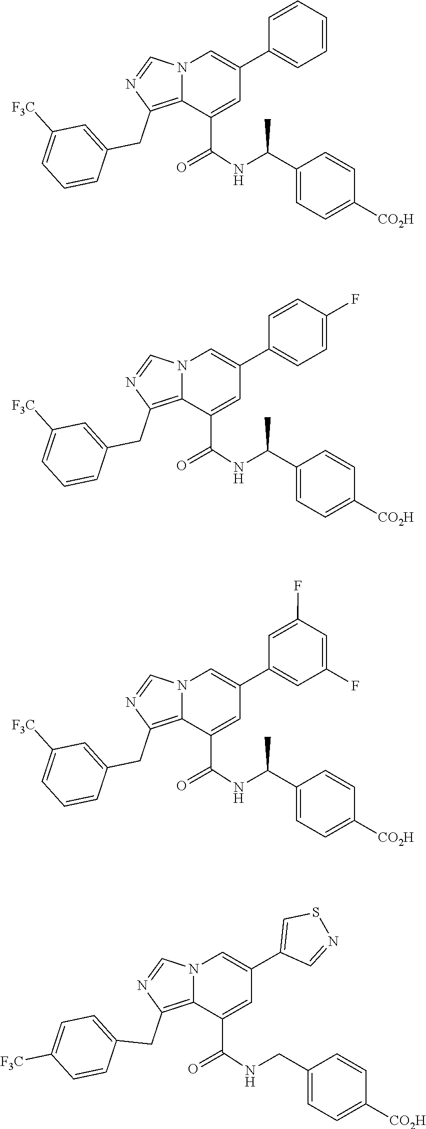 [SMILES: C[C@H](NC(=O)c1cc(-c2ccccc2)cn2cnc(Cc3cccc(C(F)(F)F)c3)c12)c1ccc(C(=O)O)cc1 ; C[C@H](NC(=O)c1cc(-c2ccc(F)cc2)cn2cnc(Cc3cccc(C(F)(F)F)c3)c12)c1ccc(C(=O)O)cc1 ; C[C@H](NC(=O)c1cc(-c2cc(F)cc(F)c2)cn2cnc(Cc3cccc(C(F)(F)F)c3)c12)c1ccc(C(=O)O)cc1 ; O=C(O)c1ccc(CNC(=O)c2cc(-c3cnsc3)cn3cnc(Cc4ccc(C(F)(F)F)cc4)c23)cc1]

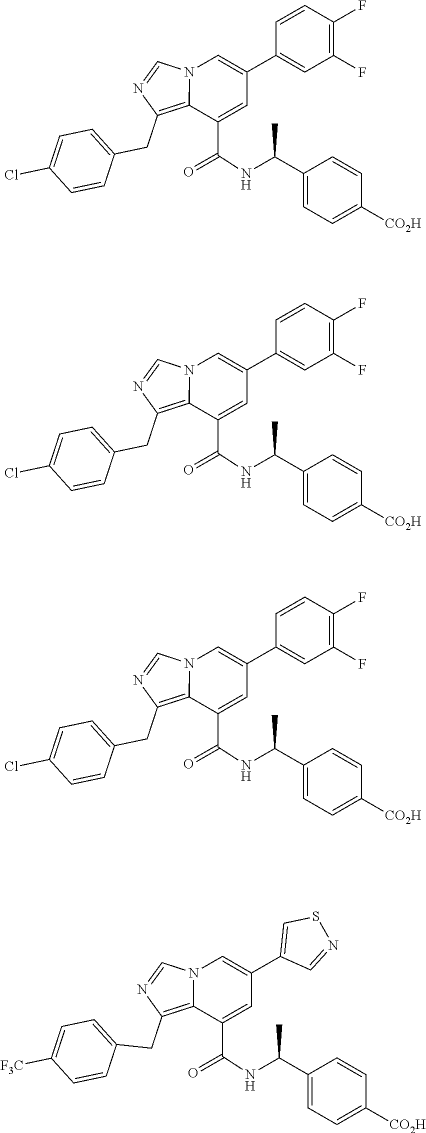 [SMILES: C[C@H](NC(=O)c1cc(-c2ccc(F)c(F)c2)cn2cnc(Cc3ccc(Cl)cc3)c12)c1ccc(C(=O)O)cc1 ; C[C@H](NC(=O)c1cc(-c2ccc(F)c(F)c2)cn2cnc(Cc3ccc(Cl)cc3)c12)c1ccc(C(=O)O)cc1 ; C[C@H](NC(=O)c1cc(-c2ccc(F)c(F)c2)cn2cnc(Cc3ccc(Cl)cc3)c12)c1ccc(C(=O)O)cc1 ; C[C@H](NC(=O)c1cc(-c2cnsc2)cn2cnc(Cc3ccc(C(F)(F)F)cc3)c12)c1ccc(C(=O)O)cc1]

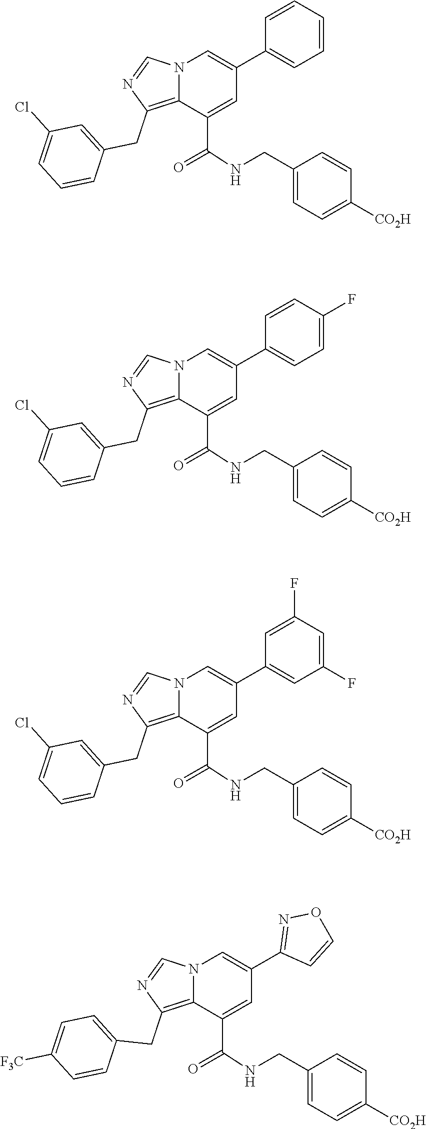 [SMILES: O=C(O)c1ccc(CNC(=O)c2cc(-c3ccccc3)cn3cnc(Cc4cccc(Cl)c4)c23)cc1 ; O=C(O)c1ccc(CNC(=O)c2cc(-c3ccc(F)cc3)cn3cnc(Cc4cccc(Cl)c4)c23)cc1 ; O=C(O)c1ccc(CNC(=O)c2cc(-c3cc(F)cc(F)c3)cn3cnc(Cc4cccc(Cl)c4)c23)cc1 ; O=C(O)c1ccc(CNC(=O)c2cc(-c3ccon3)cn3cnc(Cc4ccc(C(F)(F)F)cc4)c23)cc1]

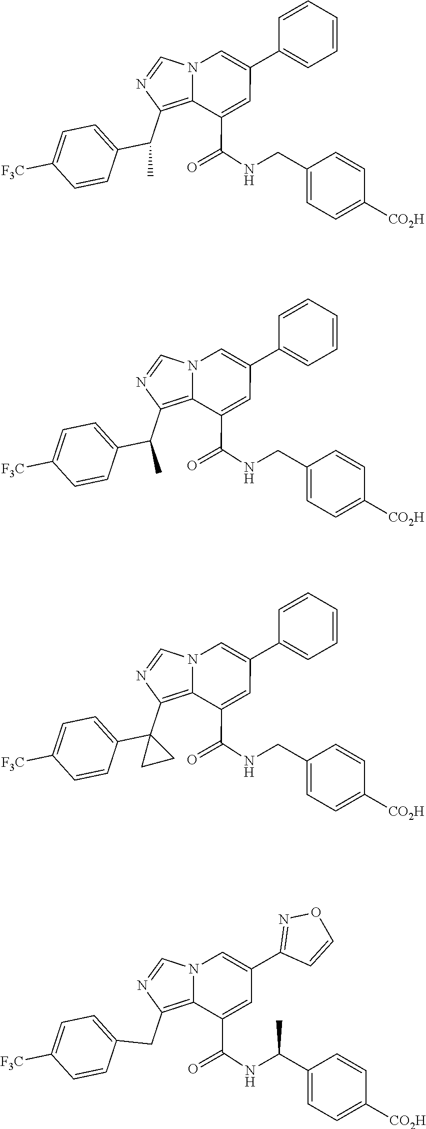 [SMILES: C[C@H](c1ccc(C(F)(F)F)cc1)c1ncn2cc(-c3ccccc3)cc(C(=O)NCc3ccc(C(=O)O)cc3)c12 ; C[C@@H](c1ccc(C(F)(F)F)cc1)c1ncn2cc(-c3ccccc3)cc(C(=O)NCc3ccc(C(=O)O)cc3)c12 ; O=C(O)c1ccc(CNC(=O)c2cc(-c3ccccc3)cn3cnc(C4(c5ccc(C(F)(F)F)cc5)CC4)c23)cc1 ; C[C@H](NC(=O)c1cc(-c2ccon2)cn2cnc(Cc3ccc(C(F)(F)F)cc3)c12)c1ccc(C(=O)O)cc1]

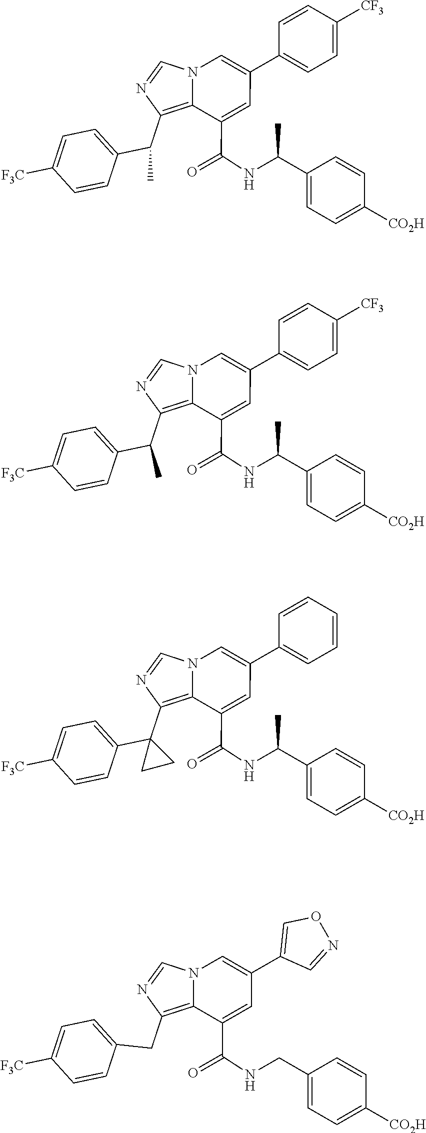 [SMILES: C[C@H](c1ccc(C(F)(F)F)cc1)c1ncn2cc(-c3ccc(C(F)(F)F)cc3)cc(C(=O)N[C@@H](C)c3ccc(C(=O)O)cc3)c12 ; C[C@@H](c1ccc(C(F)(F)F)cc1)c1ncn2cc(-c3ccc(C(F)(F)F)cc3)cc(C(=O)N[C@@H](C)c3ccc(C(=O)O)cc3)c12 ; C[C@H](NC(=O)c1cc(-c2ccccc2)cn2cnc(C3(c4ccc(C(F)(F)F)cc4)CC3)c12)c1ccc(C(=O)O)cc1 ; O=C(O)c1ccc(CNC(=O)c2cc(-c3cnoc3)cn3cnc(Cc4ccc(C(F)(F)F)cc4)c23)cc1]

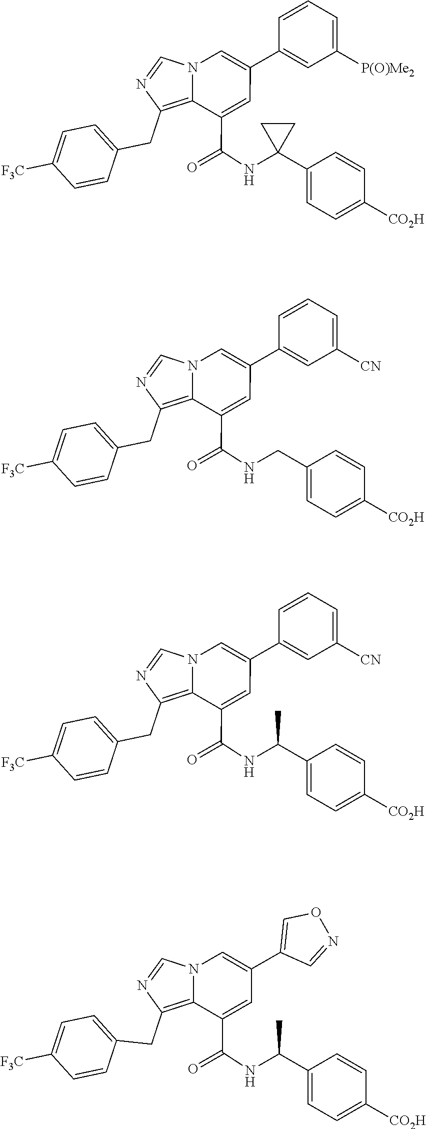

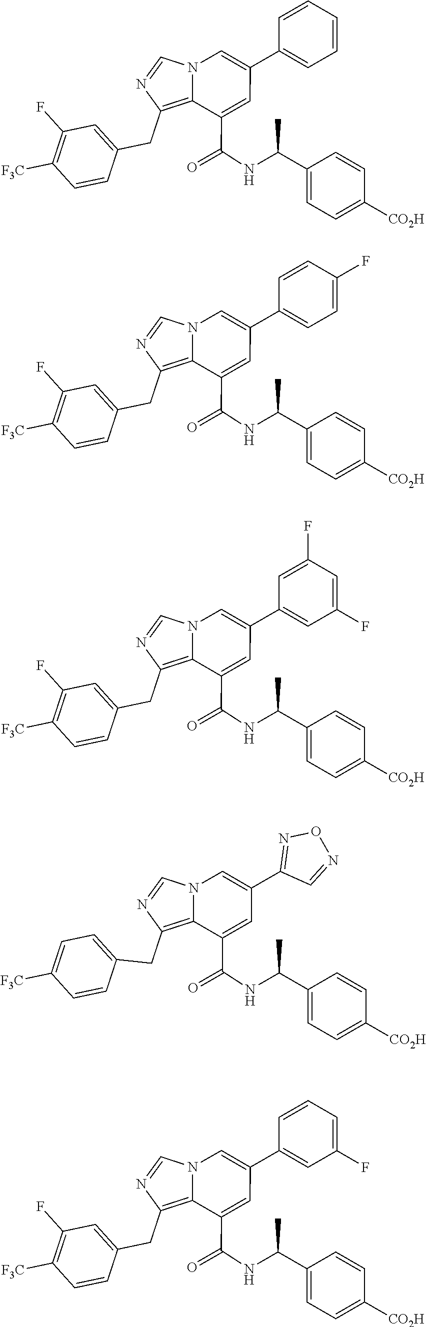 [SMILES: C[C@H](NC(=O)c1cc(-c2ccccc2)cn2cnc(Cc3ccc(C(F)(F)F)c(F)c3)c12)c1ccc(C(=O)O)cc1 ; C[C@H](NC(=O)c1cc(-c2ccc(F)cc2)cn2cnc(Cc3ccc(C(F)(F)F)c(F)c3)c12)c1ccc(C(=O)O)cc1 ; C[C@H](NC(=O)c1cc(-c2cc(F)cc(F)c2)cn2cnc(Cc3ccc(C(F)(F)F)c(F)c3)c12)c1ccc(C(=O)O)cc1 ; C[C@H](NC(=O)c1cc(-c2cnon2)cn2cnc(Cc3ccc(C(F)(F)F)cc3)c12)c1ccc(C(=O)O)cc1 ; C[C@H](NC(=O)c1cc(-c2cccc(F)c2)cn2cnc(Cc3ccc(C(F)(F)F)c(F)c3)c12)c1ccc(C(=O)O)cc1]

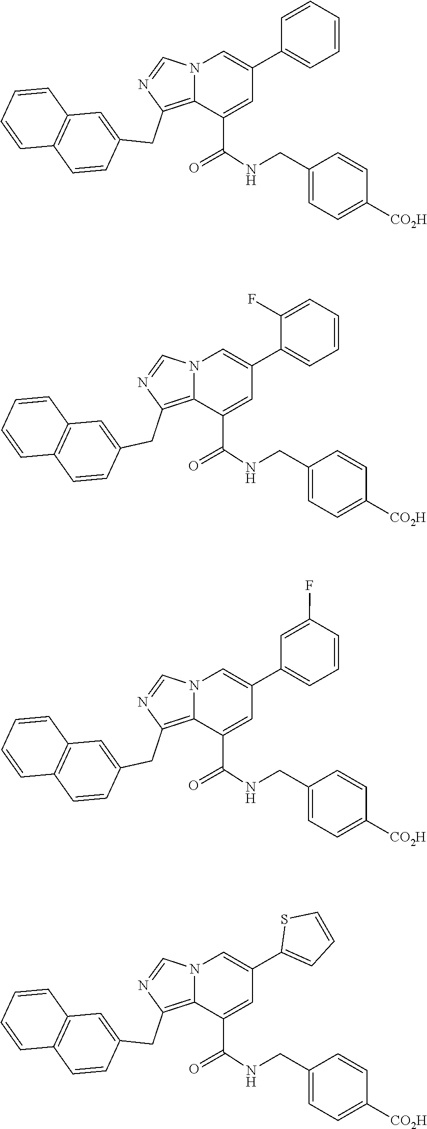 [SMILES: O=C(O)c1ccc(CNC(=O)c2cc(-c3ccccc3)cn3cnc(Cc4ccc5ccccc5c4)c23)cc1 ; O=C(O)c1ccc(CNC(=O)c2cc(-c3ccccc3F)cn3cnc(Cc4ccc5ccccc5c4)c23)cc1 ; O=C(O)c1ccc(CNC(=O)c2cc(-c3cccc(F)c3)cn3cnc(Cc4ccc5ccccc5c4)c23)cc1 ; O=C(O)c1ccc(CNC(=O)c2cc(-c3cccs3)cn3cnc(Cc4ccc5ccccc5c4)c23)cc1]

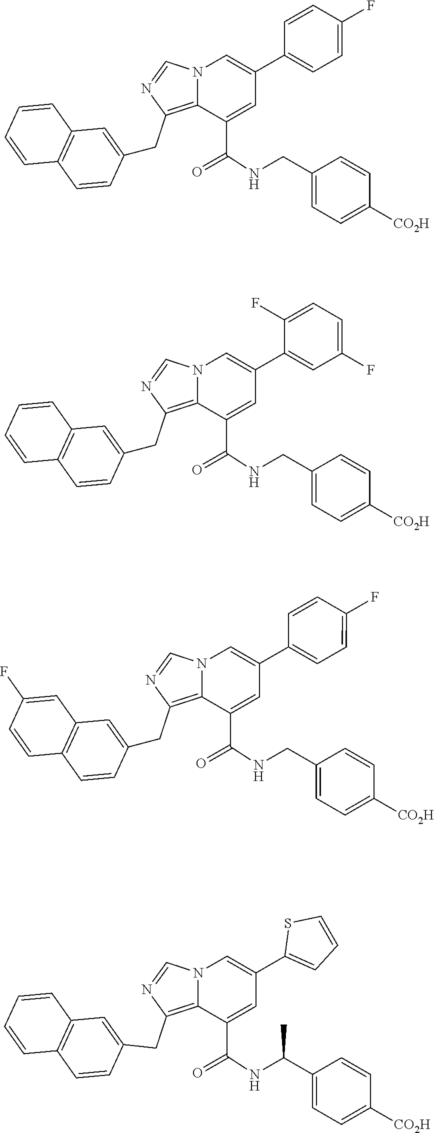 [SMILES: O=C(O)c1ccc(CNC(=O)c2cc(-c3ccc(F)cc3)cn3cnc(Cc4ccc5ccccc5c4)c23)cc1 ; O=C(O)c1ccc(CNC(=O)c2cc(-c3cc(F)ccc3F)cn3cnc(Cc4ccc5ccccc5c4)c23)cc1 ; O=C(O)c1ccc(CNC(=O)c2cc(-c3ccc(F)cc3)cn3cnc(Cc4ccc5ccc(F)cc5c4)c23)cc1 ; C[C@H](NC(=O)c1cc(-c2cccs2)cn2cnc(Cc3ccc4ccccc4c3)c12)c1ccc(C(=O)O)cc1]

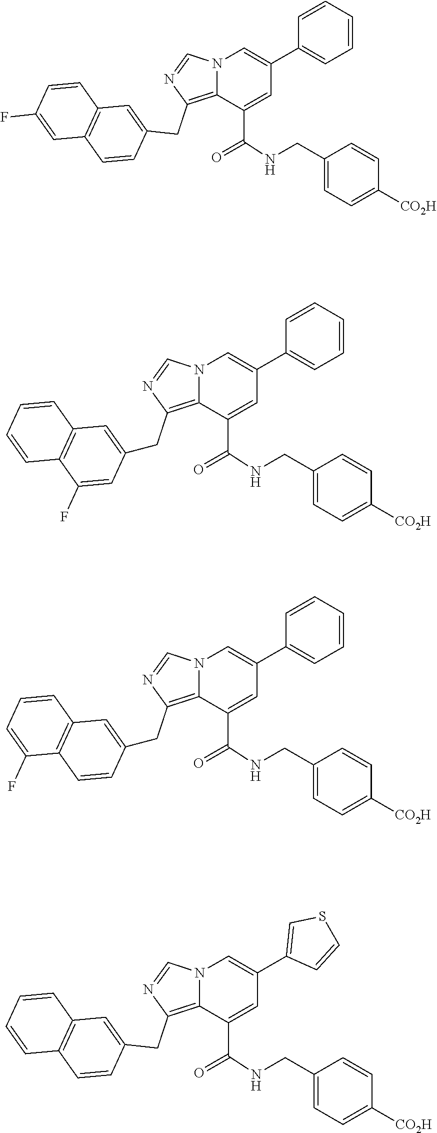 [SMILES: O=C(O)c1ccc(CNC(=O)c2cc(-c3ccccc3)cn3cnc(Cc4ccc5cc(F)ccc5c4)c23)cc1 ; O=C(O)c1ccc(CNC(=O)c2cc(-c3ccccc3)cn3cnc(Cc4cc(F)c5ccccc5c4)c23)cc1 ; O=C(O)c1ccc(CNC(=O)c2cc(-c3ccccc3)cn3cnc(Cc4ccc5c(F)cccc5c4)c23)cc1 ; O=C(O)c1ccc(CNC(=O)c2cc(-c3ccsc3)cn3cnc(Cc4ccc5ccccc5c4)c23)cc1]

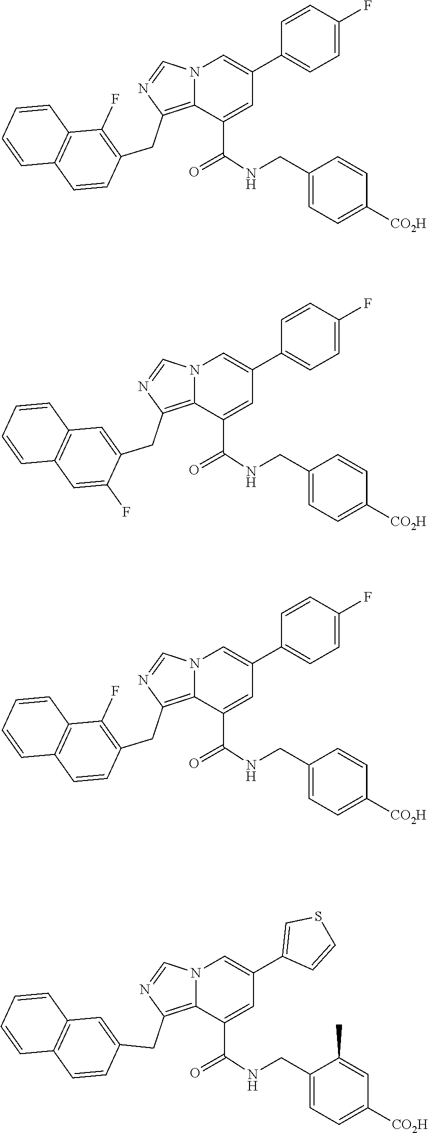

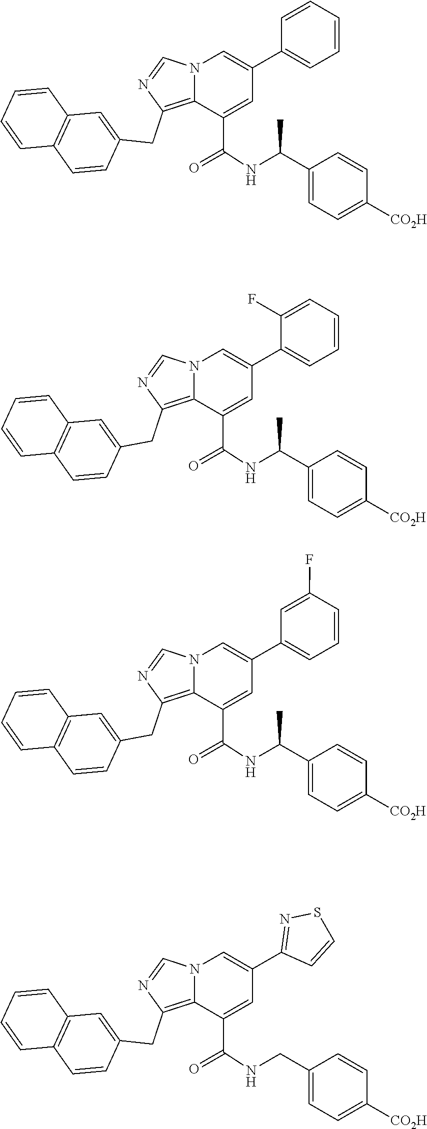 [SMILES: C[C@H](NC(=O)c1cc(-c2ccccc2)cn2cnc(Cc3ccc4ccccc4c3)c12)c1ccc(C(=O)O)cc1 ; C[C@H](NC(=O)c1cc(-c2ccccc2F)cn2cnc(Cc3ccc4ccccc4c3)c12)c1ccc(C(=O)O)cc1 ; C[C@H](NC(=O)c1cc(-c2cccc(F)c2)cn2cnc(Cc3ccc4ccccc4c3)c12)c1ccc(C(=O)O)cc1 ; O=C(O)c1ccc(CNC(=O)c2cc(-c3ccsn3)cn3cnc(Cc4ccc5ccccc5c4)c23)cc1]

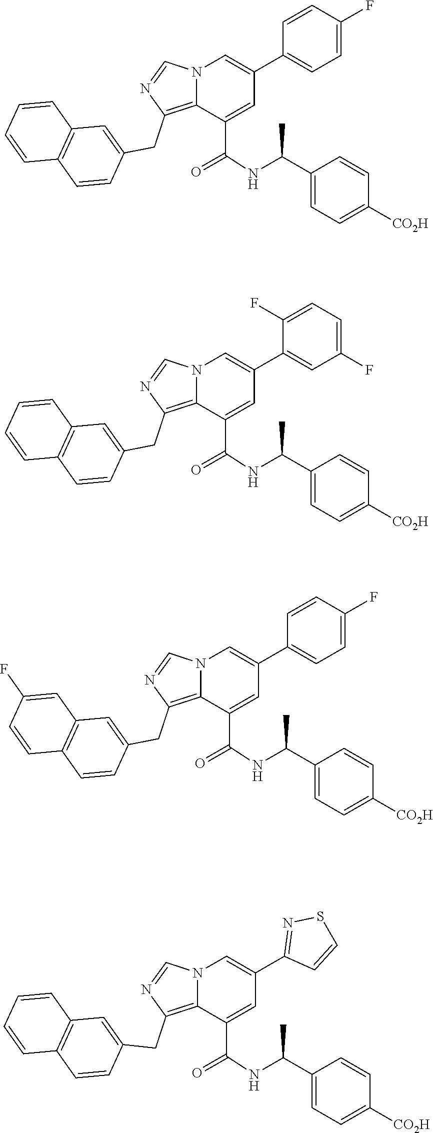 [SMILES: C[C@H](NC(=O)c1cc(-c2ccc(F)cc2)cn2cnc(Cc3ccc4ccccc4c3)c12)c1ccc(C(=O)O)cc1 ; C[C@H](NC(=O)c1cc(-c2cc(F)ccc2F)cn2cnc(Cc3ccc4ccccc4c3)c12)c1ccc(C(=O)O)cc1 ; C[C@H](NC(=O)c1cc(-c2ccc(F)cc2)cn2cnc(Cc3ccc4ccc(F)cc4c3)c12)c1ccc(C(=O)O)cc1 ; C[C@H](NC(=O)c1cc(-c2ccsn2)cn2cnc(Cc3ccc4ccccc4c3)c12)c1ccc(C(=O)O)cc1]

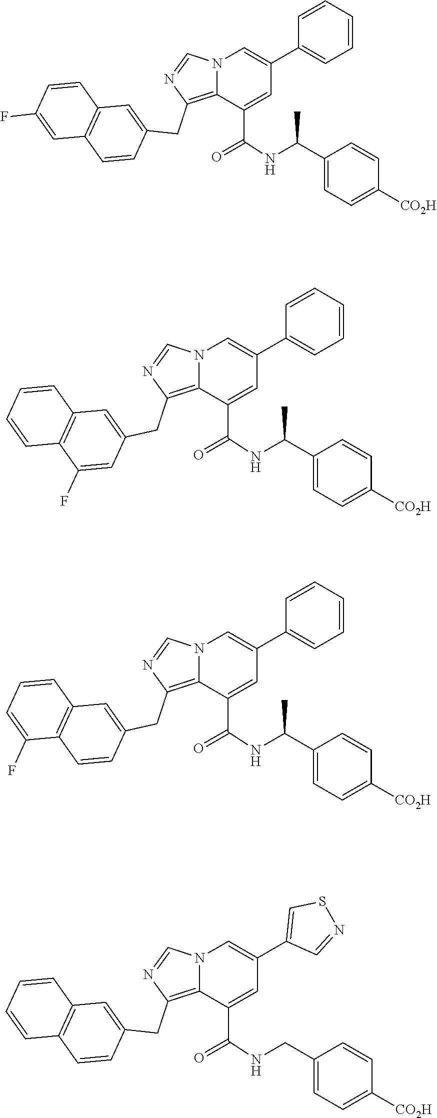 [SMILES: C[C@H](NC(=O)c1cc(-c2ccccc2)cn2cnc(Cc3ccc4cc(F)ccc4c3)c12)c1ccc(C(=O)O)cc1 ; C[C@H](NC(=O)c1cc(-c2ccccc2)cn2cnc(Cc3cc(F)c4ccccc4c3)c12)c1ccc(C(=O)O)cc1 ; C[C@H](NC(=O)c1cc(-c2ccccc2)cn2cnc(Cc3ccc4c(F)cccc4c3)c12)c1ccc(C(=O)O)cc1 ; O=C(O)c1ccc(CNC(=O)c2cc(-c3cnsc3)cn3cnc(Cc4ccc5ccccc5c4)c23)cc1]

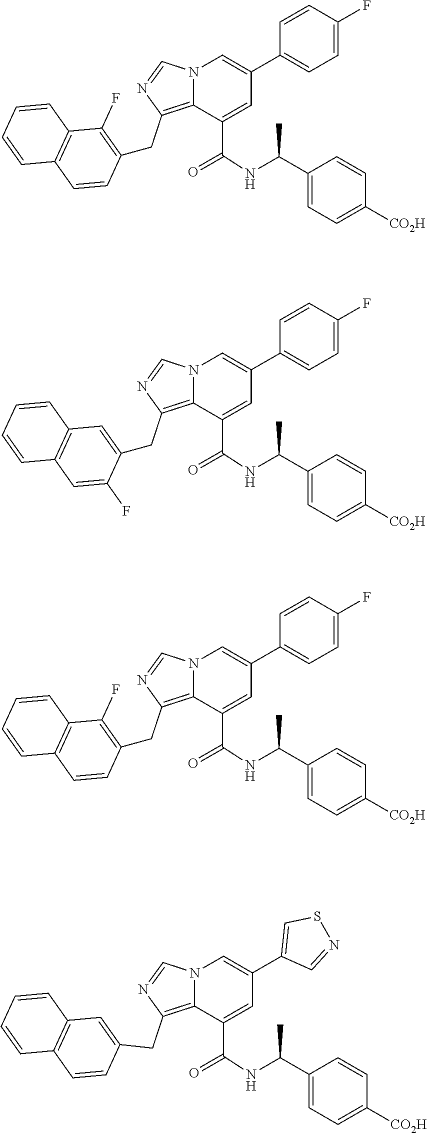 [SMILES: C[C@H](NC(=O)c1cc(-c2ccc(F)cc2)cn2cnc(Cc3ccc4ccccc4c3F)c12)c1ccc(C(=O)O)cc1 ; C[C@H](NC(=O)c1cc(-c2ccc(F)cc2)cn2cnc(Cc3cc4ccccc4cc3F)c12)c1ccc(C(=O)O)cc1 ; C[C@H](NC(=O)c1cc(-c2ccc(F)cc2)cn2cnc(Cc3ccc4ccccc4c3F)c12)c1ccc(C(=O)O)cc1 ; C[C@H](NC(=O)c1cc(-c2cnsc2)cn2cnc(Cc3ccc4ccccc4c3)c12)c1ccc(C(=O)O)cc1]

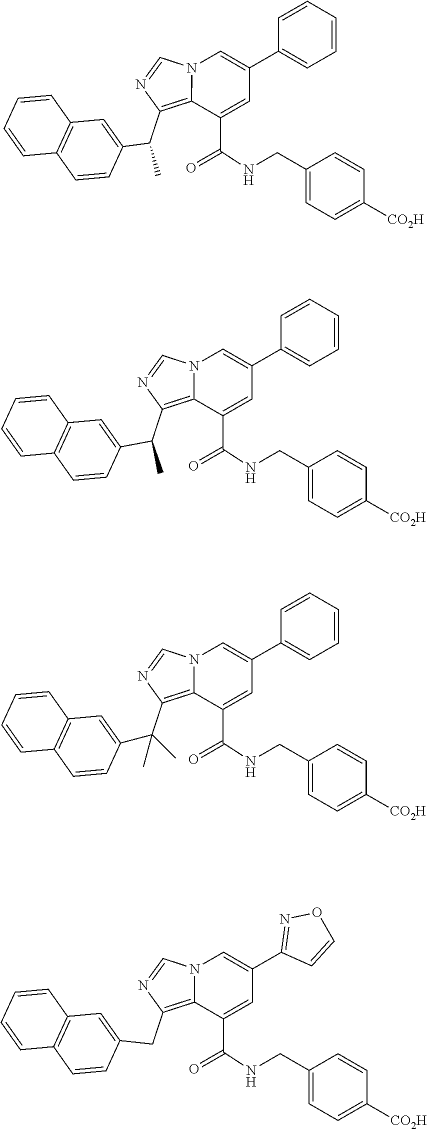 [SMILES: C[C@H](c1ccc2ccccc2c1)c1ncn2cc(-c3ccccc3)cc(C(=O)NCc3ccc(C(=O)O)cc3)c12 ; C[C@@H](c1ccc2ccccc2c1)c1ncn2cc(-c3ccccc3)cc(C(=O)NCc3ccc(C(=O)O)cc3)c12 ; CC(C)(c1ccc2ccccc2c1)c1ncn2cc(-c3ccccc3)cc(C(=O)NCc3ccc(C(=O)O)cc3)c12 ; O=C(O)c1ccc(CNC(=O)c2cc(-c3ccon3)cn3cnc(Cc4ccc5ccccc5c4)c23)cc1]

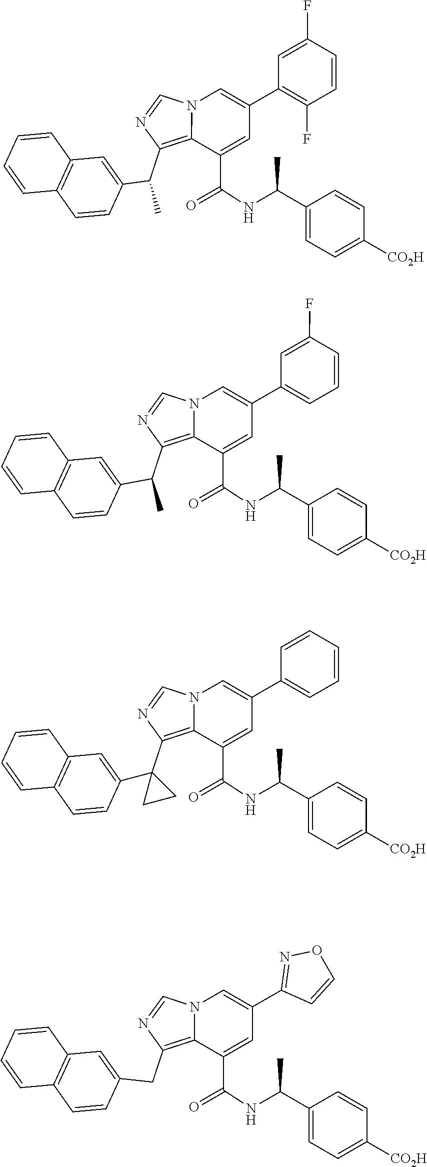 [SMILES: C[C@H](c1ccc2ccccc2c1)c1ncn2cc(-c3cc(F)ccc3F)cc(C(=O)N[C@@H](C)c3ccc(C(=O)O)cc3)c12 ; C[C@@H](c1ccc2ccccc2c1)c1ncn2cc(-c3cccc(F)c3)cc(C(=O)N[C@@H](C)c3ccc(C(=O)O)cc3)c12 ; C[C@H](NC(=O)c1cc(-c2ccccc2)cn2cnc(C3(c4ccc5ccccc5c4)CC3)c12)c1ccc(C(=O)O)cc1 ; C[C@H](NC(=O)c1cc(-c2ccon2)cn2cnc(Cc3ccc4ccccc4c3)c12)c1ccc(C(=O)O)cc1]

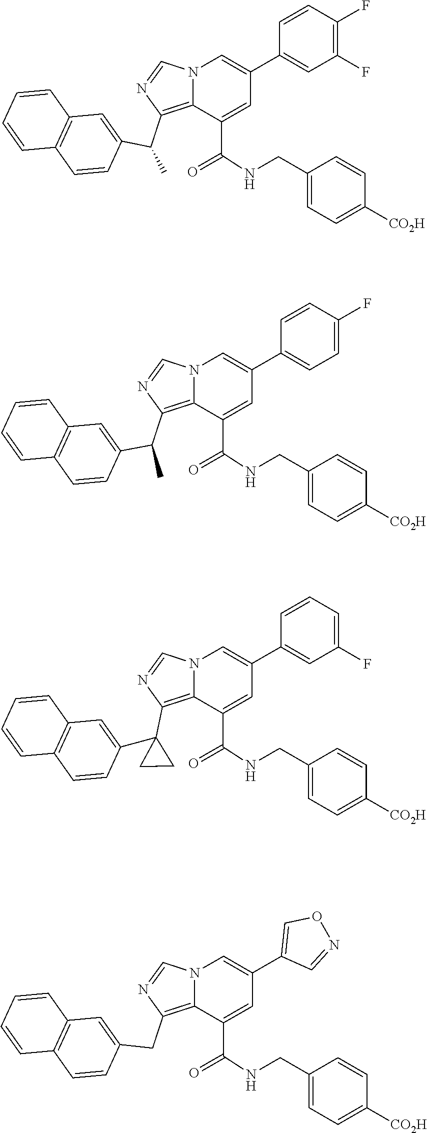 [SMILES: C[C@H](c1ccc2ccccc2c1)c1ncn2cc(-c3ccc(F)c(F)c3)cc(C(=O)NCc3ccc(C(=O)O)cc3)c12 ; C[C@@H](c1ccc2ccccc2c1)c1ncn2cc(-c3ccc(F)cc3)cc(C(=O)NCc3ccc(C(=O)O)cc3)c12 ; O=C(O)c1ccc(CNC(=O)c2cc(-c3cccc(F)c3)cn3cnc(C4(c5ccc6ccccc6c5)CC4)c23)cc1 ; O=C(O)c1ccc(CNC(=O)c2cc(-c3cnoc3)cn3cnc(Cc4ccc5ccccc5c4)c23)cc1]

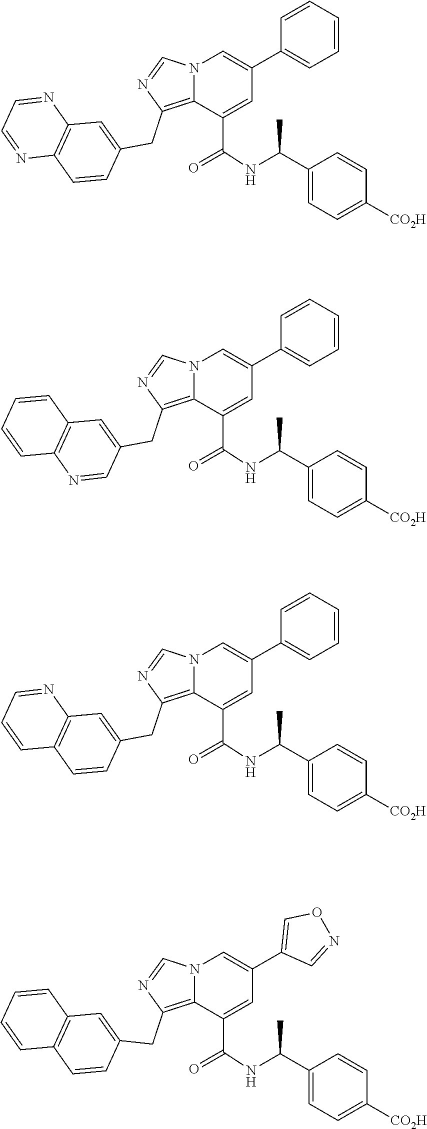 [SMILES: C[C@H](NC(=O)c1cc(-c2ccccc2)cn2cnc(Cc3ccc4nccnc4c3)c12)c1ccc(C(=O)O)cc1 ; C[C@H](NC(=O)c1cc(-c2ccccc2)cn2cnc(Cc3cnc4ccccc4c3)c12)c1ccc(C(=O)O)cc1 ; C[C@H](NC(=O)c1cc(-c2ccccc2)cn2cnc(Cc3ccc4cccnc4c3)c12)c1ccc(C(=O)O)cc1 ; C[C@H](NC(=O)c1cc(-c2cnoc2)cn2cnc(Cc3ccc4ccccc4c3)c12)c1ccc(C(=O)O)cc1]

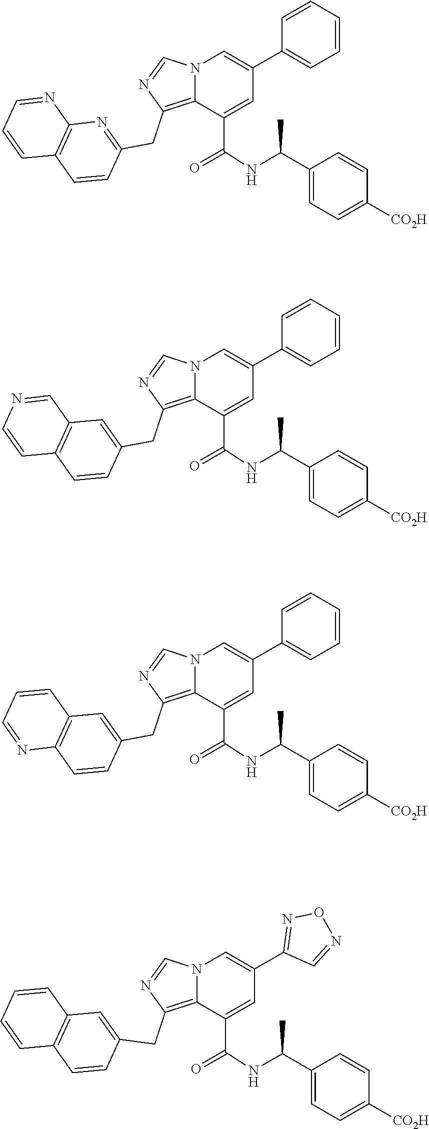 [SMILES: C[C@H](NC(=O)c1cc(-c2ccccc2)cn2cnc(Cc3ccc4cccnc4n3)c12)c1ccc(C(=O)O)cc1 ; C[C@H](NC(=O)c1cc(-c2ccccc2)cn2cnc(Cc3ccc4ccncc4c3)c12)c1ccc(C(=O)O)cc1 ; C[C@H](NC(=O)c1cc(-c2ccccc2)cn2cnc(Cc3ccc4ncccc4c3)c12)c1ccc(C(=O)O)cc1 ; C[C@H](NC(=O)c1cc(-c2cnon2)cn2cnc(Cc3ccc4ccccc4c3)c12)c1ccc(C(=O)O)cc1]

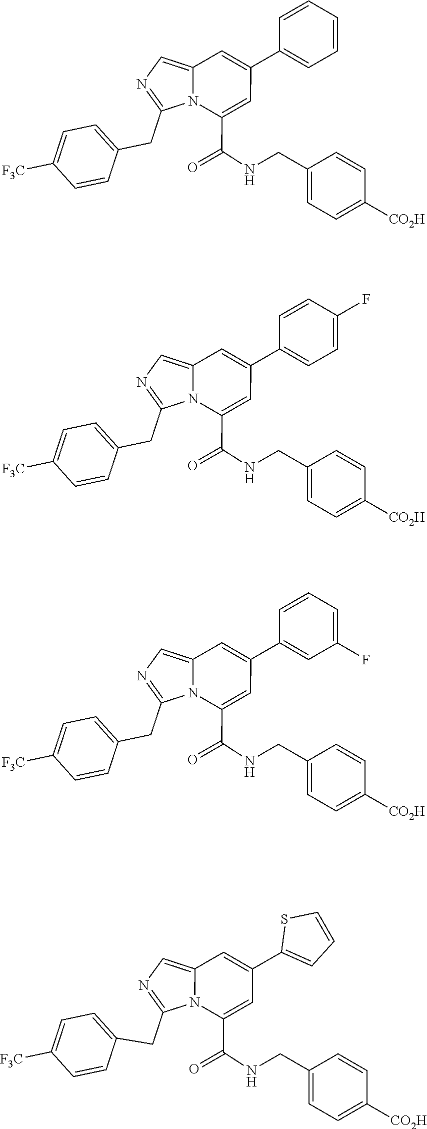 [SMILES: O=C(O)c1ccc(CNC(=O)c2cc(-c3ccccc3)cc3cnc(Cc4ccc(C(F)(F)F)cc4)n23)cc1 ; O=C(O)c1ccc(CNC(=O)c2cc(-c3ccc(F)cc3)cc3cnc(Cc4ccc(C(F)(F)F)cc4)n23)cc1 ; O=C(O)c1ccc(CNC(=O)c2cc(-c3cccc(F)c3)cc3cnc(Cc4ccc(C(F)(F)F)cc4)n23)cc1 ; O=C(O)c1ccc(CNC(=O)c2cc(-c3cccs3)cc3cnc(Cc4ccc(C(F)(F)F)cc4)n23)cc1]

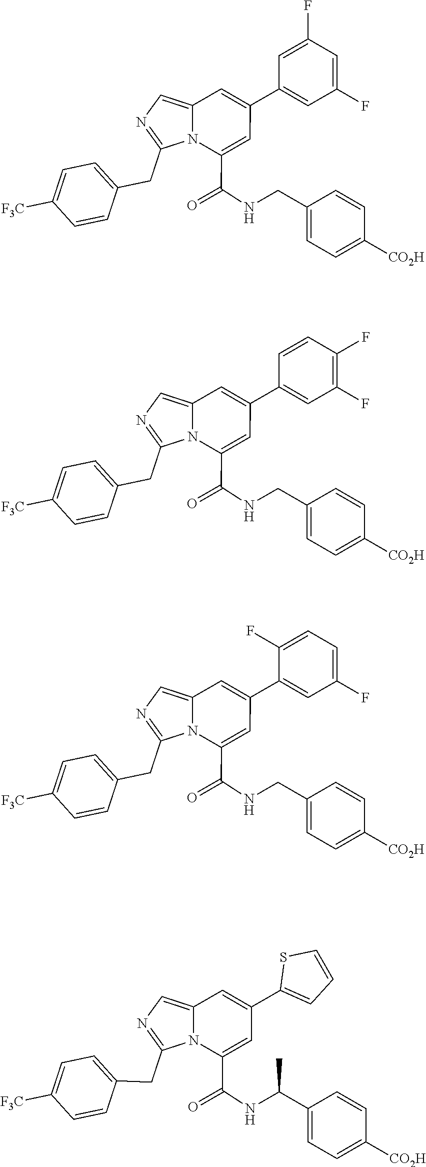 [SMILES: O=C(O)c1ccc(CNC(=O)c2cc(-c3cc(F)cc(F)c3)cc3cnc(Cc4ccc(C(F)(F)F)cc4)n23)cc1 ; O=C(O)c1ccc(CNC(=O)c2cc(-c3ccc(F)c(F)c3)cc3cnc(Cc4ccc(C(F)(F)F)cc4)n23)cc1 ; O=C(O)c1ccc(CNC(=O)c2cc(-c3cc(F)ccc3F)cc3cnc(Cc4ccc(C(F)(F)F)cc4)n23)cc1 ; C[C@H](NC(=O)c1cc(-c2cccs2)cc2cnc(Cc3ccc(C(F)(F)F)cc3)n12)c1ccc(C(=O)O)cc1]

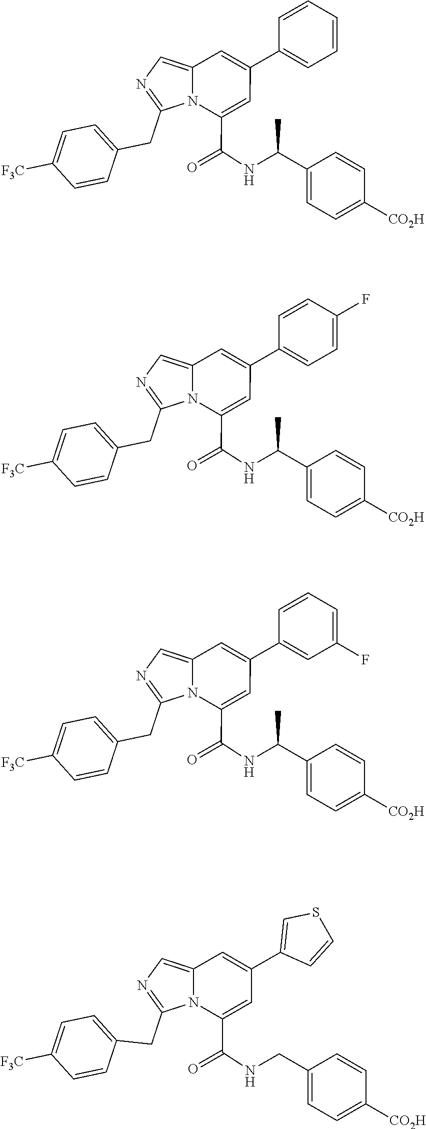 [SMILES: C[C@H](NC(=O)c1cc(-c2ccccc2)cc2cnc(Cc3ccc(C(F)(F)F)cc3)n12)c1ccc(C(=O)O)cc1 ; C[C@H](NC(=O)c1cc(-c2ccc(F)cc2)cc2cnc(Cc3ccc(C(F)(F)F)cc3)n12)c1ccc(C(=O)O)cc1 ; C[C@H](NC(=O)c1cc(-c2cccc(F)c2)cc2cnc(Cc3ccc(C(F)(F)F)cc3)n12)c1ccc(C(=O)O)cc1 ; O=C(O)c1ccc(CNC(=O)c2cc(-c3ccsc3)cc3cnc(Cc4ccc(C(F)(F)F)cc4)n23)cc1]

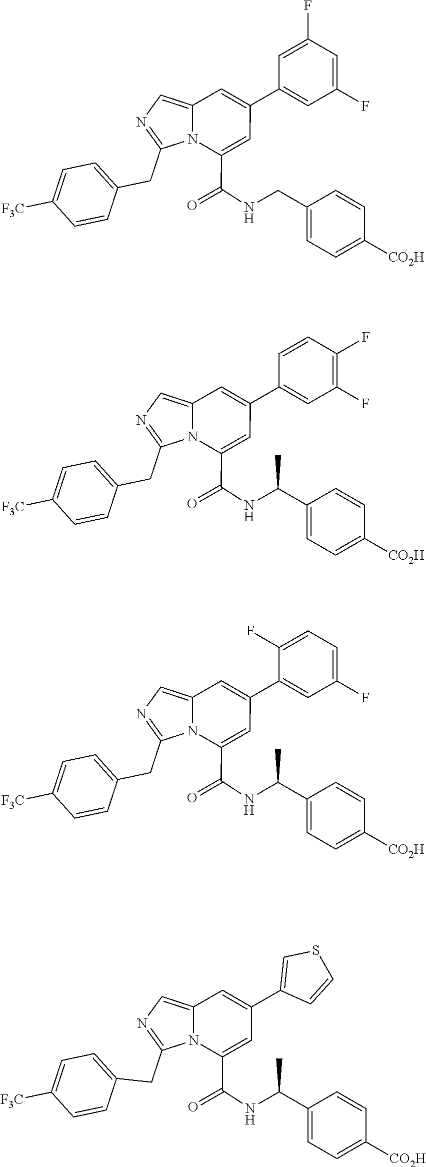 [SMILES: O=C(O)c1ccc(CNC(=O)c2cc(-c3cc(F)cc(F)c3)cc3cnc(Cc4ccc(C(F)(F)F)cc4)n23)cc1 ; C[C@H](NC(=O)c1cc(-c2ccc(F)c(F)c2)cc2cnc(Cc3ccc(C(F)(F)F)cc3)n12)c1ccc(C(=O)O)cc1 ; C[C@H](NC(=O)c1cc(-c2cc(F)ccc2F)cc2cnc(Cc3ccc(C(F)(F)F)cc3)n12)c1ccc(C(=O)O)cc1 ; C[C@H](NC(=O)c1cc(-c2ccsc2)cc2cnc(Cc3ccc(C(F)(F)F)cc3)n12)c1ccc(C(=O)O)cc1]

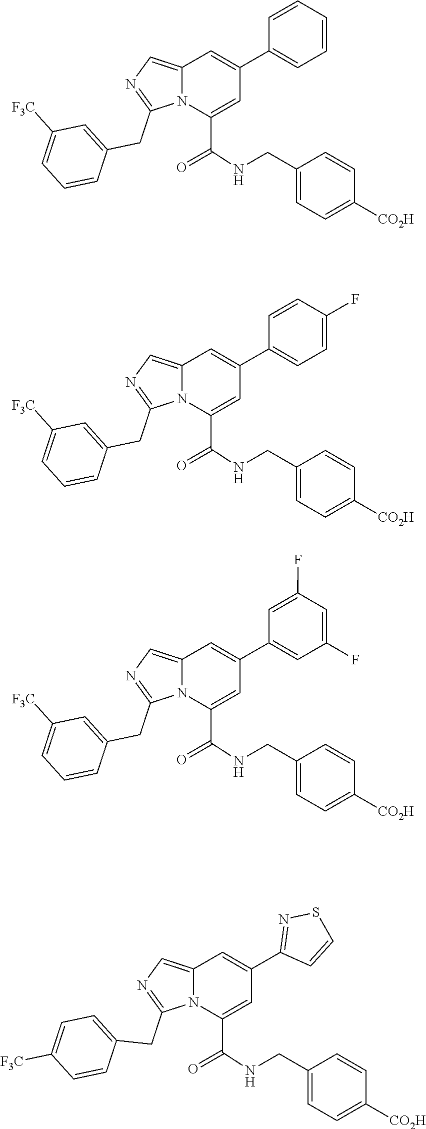 [SMILES: O=C(O)c1ccc(CNC(=O)c2cc(-c3ccccc3)cc3cnc(Cc4cccc(C(F)(F)F)c4)n23)cc1 ; O=C(O)c1ccc(CNC(=O)c2cc(-c3ccc(F)cc3)cc3cnc(Cc4cccc(C(F)(F)F)c4)n23)cc1 ; O=C(O)c1ccc(CNC(=O)c2cc(-c3cc(F)cc(F)c3)cc3cnc(Cc4cccc(C(F)(F)F)c4)n23)cc1 ; O=C(O)c1ccc(CNC(=O)c2cc(-c3ccsn3)cc3cnc(Cc4ccc(C(F)(F)F)cc4)n23)cc1]

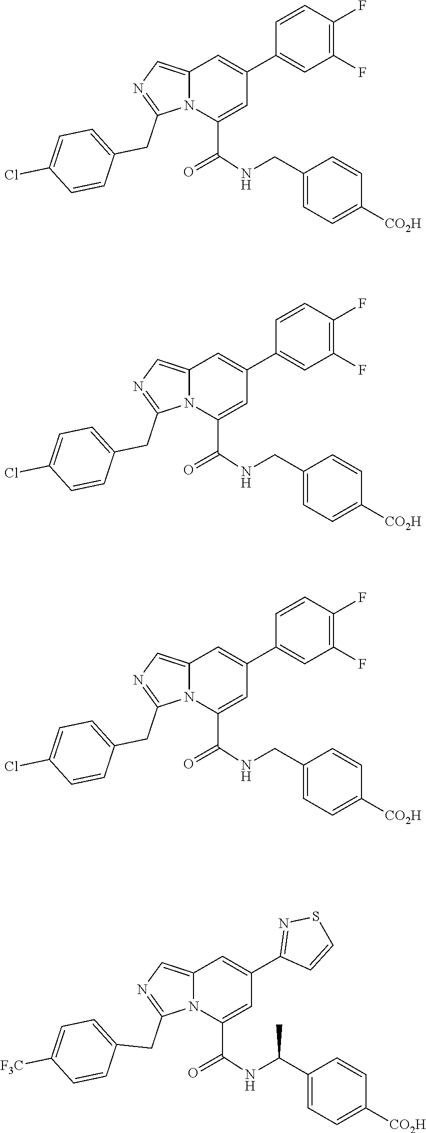 [SMILES: O=C(O)c1ccc(CNC(=O)c2cc(-c3ccc(F)c(F)c3)cc3cnc(Cc4ccc(Cl)cc4)n23)cc1 ; O=C(O)c1ccc(CNC(=O)c2cc(-c3ccc(F)c(F)c3)cc3cnc(Cc4ccc(Cl)cc4)n23)cc1 ; O=C(O)c1ccc(CNC(=O)c2cc(-c3ccc(F)c(F)c3)cc3cnc(Cc4ccc(Cl)cc4)n23)cc1 ; C[C@H](NC(=O)c1cc(-c2ccsn2)cc2cnc(Cc3ccc(C(F)(F)F)cc3)n12)c1ccc(C(=O)O)cc1]

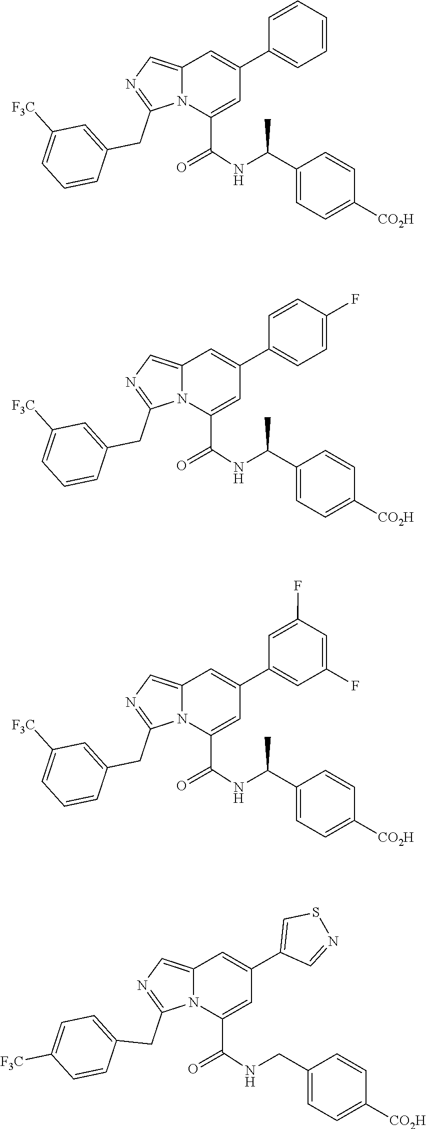 [SMILES: C[C@H](NC(=O)c1cc(-c2ccccc2)cc2cnc(Cc3cccc(C(F)(F)F)c3)n12)c1ccc(C(=O)O)cc1 ; C[C@H](NC(=O)c1cc(-c2ccc(F)cc2)cc2cnc(Cc3cccc(C(F)(F)F)c3)n12)c1ccc(C(=O)O)cc1 ; C[C@H](NC(=O)c1cc(-c2cc(F)cc(F)c2)cc2cnc(Cc3cccc(C(F)(F)F)c3)n12)c1ccc(C(=O)O)cc1 ; O=C(O)c1ccc(CNC(=O)c2cc(-c3cnsc3)cc3cnc(Cc4ccc(C(F)(F)F)cc4)n23)cc1]

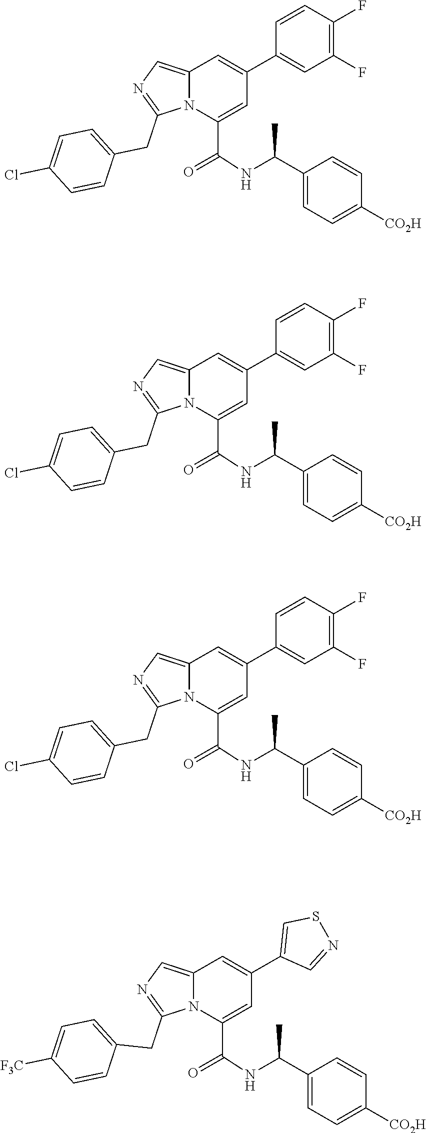 [SMILES: C[C@H](NC(=O)c1cc(-c2ccc(F)c(F)c2)cc2cnc(Cc3ccc(Cl)cc3)n12)c1ccc(C(=O)O)cc1 ; C[C@H](NC(=O)c1cc(-c2ccc(F)c(F)c2)cc2cnc(Cc3ccc(Cl)cc3)n12)c1ccc(C(=O)O)cc1 ; C[C@H](NC(=O)c1cc(-c2ccc(F)c(F)c2)cc2cnc(Cc3ccc(Cl)cc3)n12)c1ccc(C(=O)O)cc1 ; C[C@H](NC(=O)c1cc(-c2cnsc2)cc2cnc(Cc3ccc(C(F)(F)F)cc3)n12)c1ccc(C(=O)O)cc1]

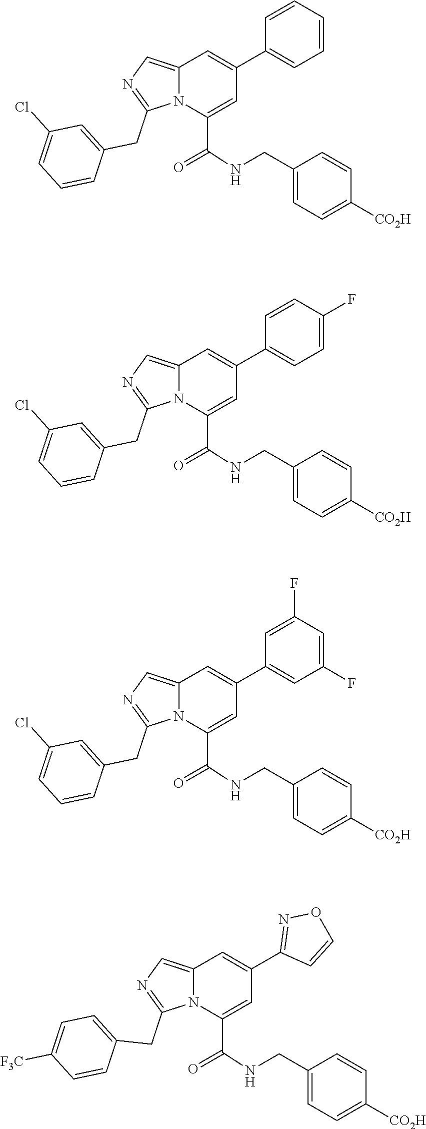 [SMILES: O=C(O)c1ccc(CNC(=O)c2cc(-c3ccccc3)cc3cnc(Cc4cccc(Cl)c4)n23)cc1 ; O=C(O)c1ccc(CNC(=O)c2cc(-c3ccc(F)cc3)cc3cnc(Cc4cccc(Cl)c4)n23)cc1 ; O=C(O)c1ccc(CNC(=O)c2cc(-c3cc(F)cc(F)c3)cc3cnc(Cc4cccc(Cl)c4)n23)cc1 ; O=C(O)c1ccc(CNC(=O)c2cc(-c3ccon3)cc3cnc(Cc4ccc(C(F)(F)F)cc4)n23)cc1]

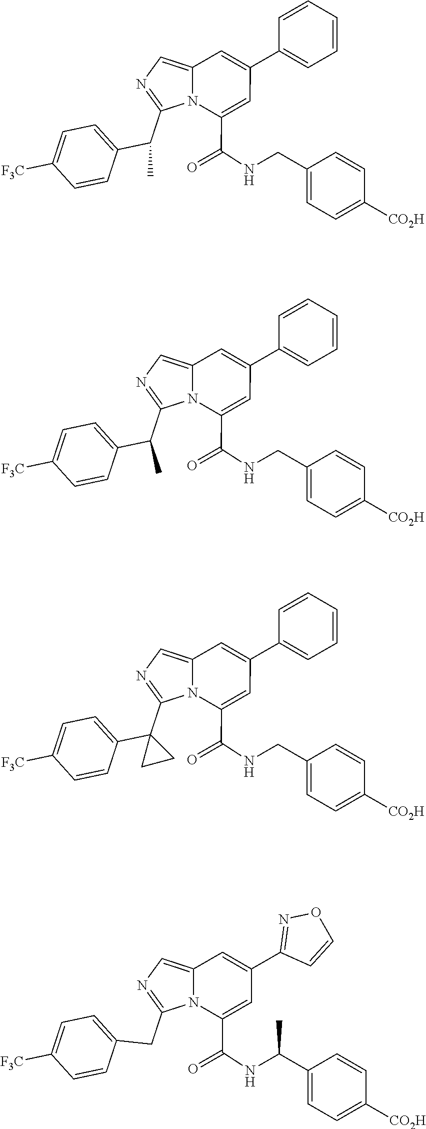 [SMILES: C[C@H](c1ccc(C(F)(F)F)cc1)c1ncc2cc(-c3ccccc3)cc(C(=O)NCc3ccc(C(=O)O)cc3)n12 ; C[C@@H](c1ccc(C(F)(F)F)cc1)c1ncc2cc(-c3ccccc3)cc(C(=O)NCc3ccc(C(=O)O)cc3)n12 ; O=C(O)c1ccc(CNC(=O)c2cc(-c3ccccc3)cc3cnc(C4(c5ccc(C(F)(F)F)cc5)CC4)n23)cc1 ; C[C@H](NC(=O)c1cc(-c2ccon2)cc2cnc(Cc3ccc(C(F)(F)F)cc3)n12)c1ccc(C(=O)O)cc1]

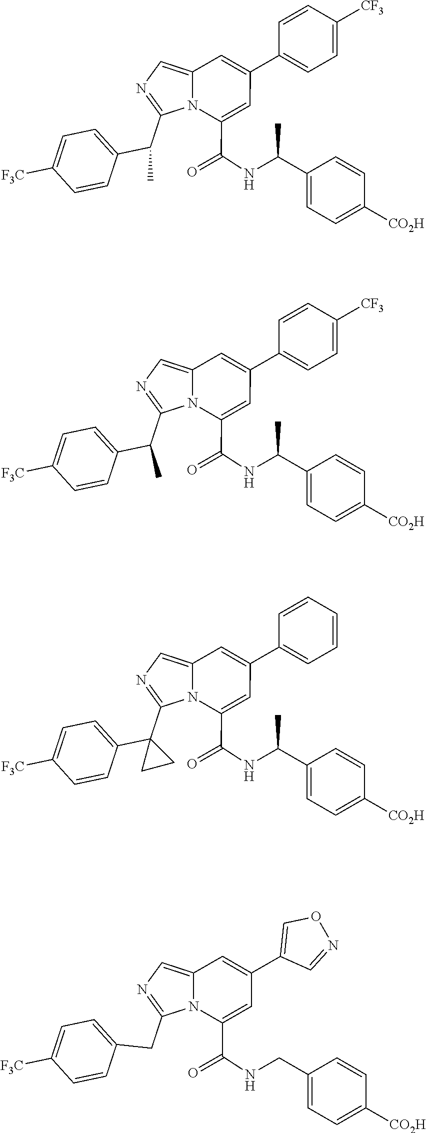 [SMILES: C[C@H](c1ccc(C(F)(F)F)cc1)c1ncc2cc(-c3ccc(C(F)(F)F)cc3)cc(C(=O)N[C@@H](C)c3ccc(C(=O)O)cc3)n12 ; C[C@@H](c1ccc(C(F)(F)F)cc1)c1ncc2cc(-c3ccc(C(F)(F)F)cc3)cc(C(=O)N[C@@H](C)c3ccc(C(=O)O)cc3)n12 ; C[C@H](NC(=O)c1cc(-c2ccccc2)cc2cnc(C3(c4ccc(C(F)(F)F)cc4)CC3)n12)c1ccc(C(=O)O)cc1 ; O=C(O)c1ccc(CNC(=O)c2cc(-c3cnoc3)cc3cnc(Cc4ccc(C(F)(F)F)cc4)n23)cc1]

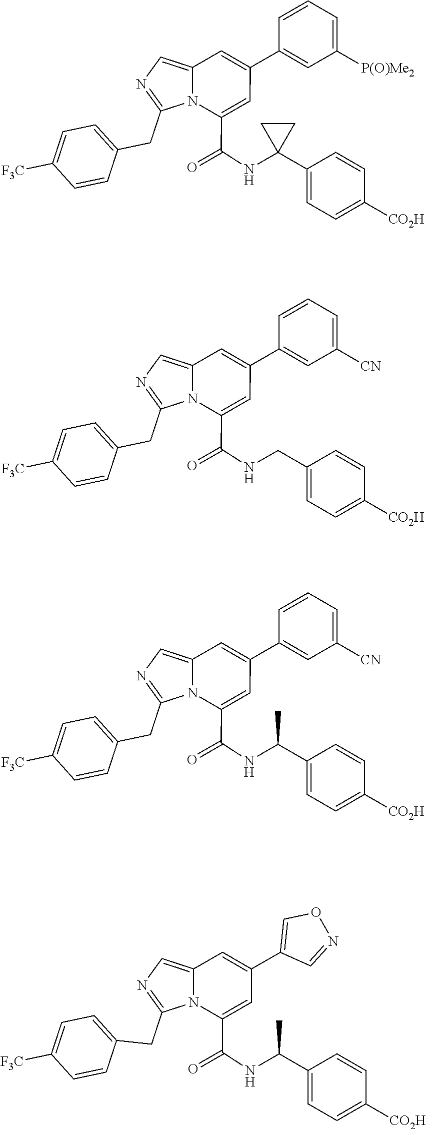

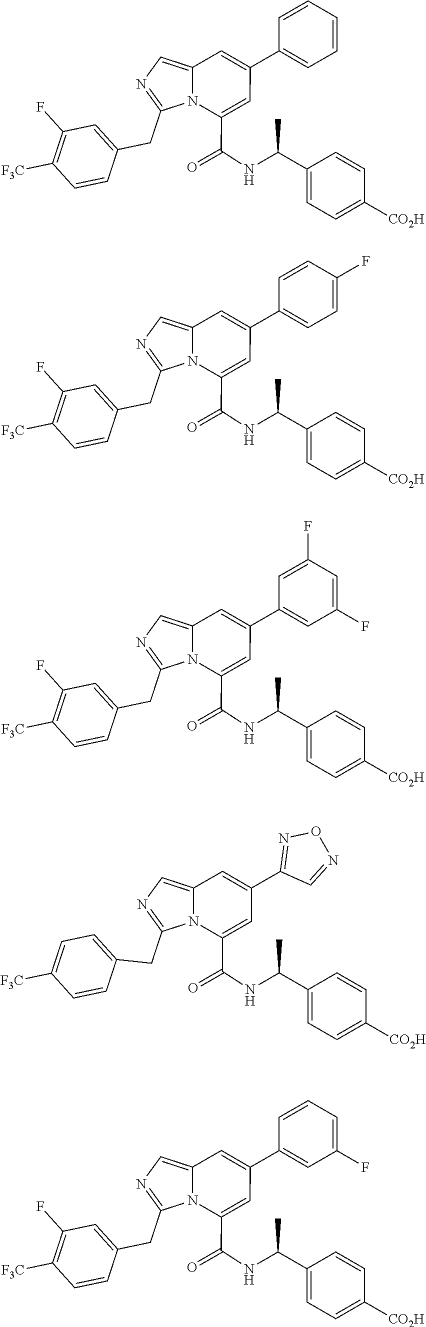 [SMILES: C[C@H](NC(=O)c1cc(-c2ccccc2)cc2cnc(Cc3ccc(C(F)(F)F)c(F)c3)n12)c1ccc(C(=O)O)cc1 ; C[C@H](NC(=O)c1cc(-c2ccc(F)cc2)cc2cnc(Cc3ccc(C(F)(F)F)c(F)c3)n12)c1ccc(C(=O)O)cc1 ; C[C@H](NC(=O)c1cc(-c2cc(F)cc(F)c2)cc2cnc(Cc3ccc(C(F)(F)F)c(F)c3)n12)c1ccc(C(=O)O)cc1 ; C[C@H](NC(=O)c1cc(-c2cnon2)cc2cnc(Cc3ccc(C(F)(F)F)cc3)n12)c1ccc(C(=O)O)cc1 ; C[C@H](NC(=O)c1cc(-c2cccc(F)c2)cc2cnc(Cc3ccc(C(F)(F)F)c(F)c3)n12)c1ccc(C(=O)O)cc1]

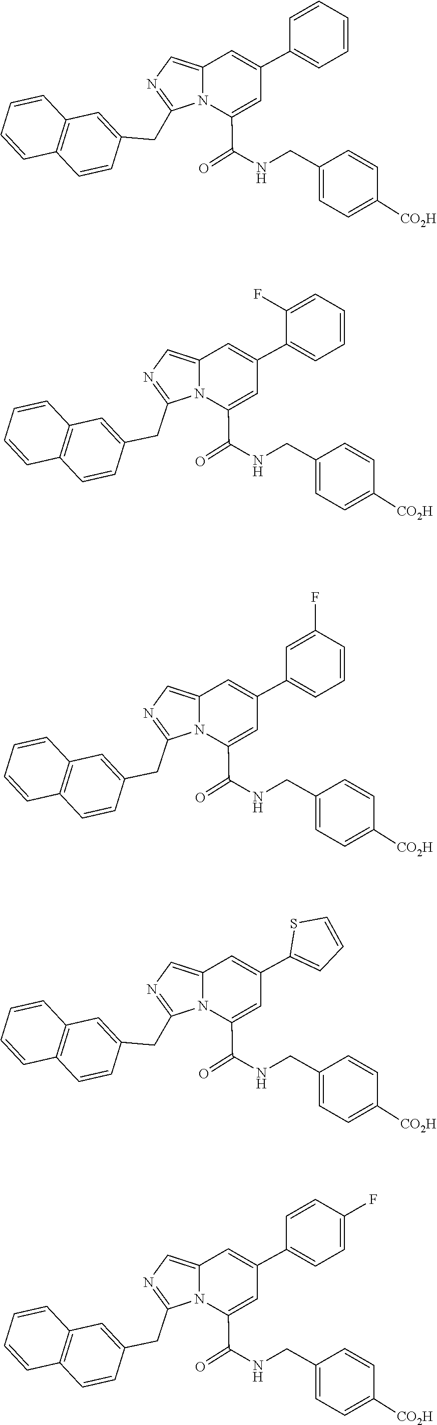 [SMILES: O=C(O)c1ccc(CNC(=O)c2cc(-c3ccccc3)cc3cnc(Cc4ccc5ccccc5c4)n23)cc1 ; O=C(O)c1ccc(CNC(=O)c2cc(-c3ccccc3F)cc3cnc(Cc4ccc5ccccc5c4)n23)cc1 ; O=C(O)c1ccc(CNC(=O)c2cc(-c3cccc(F)c3)cc3cnc(Cc4ccc5ccccc5c4)n23)cc1 ; O=C(O)c1ccc(CNC(=O)c2cc(-c3cccs3)cc3cnc(Cc4ccc5ccccc5c4)n23)cc1 ; O=C(O)c1ccc(CNC(=O)c2cc(-c3ccc(F)cc3)cc3cnc(Cc4ccc5ccccc5c4)n23)cc1]

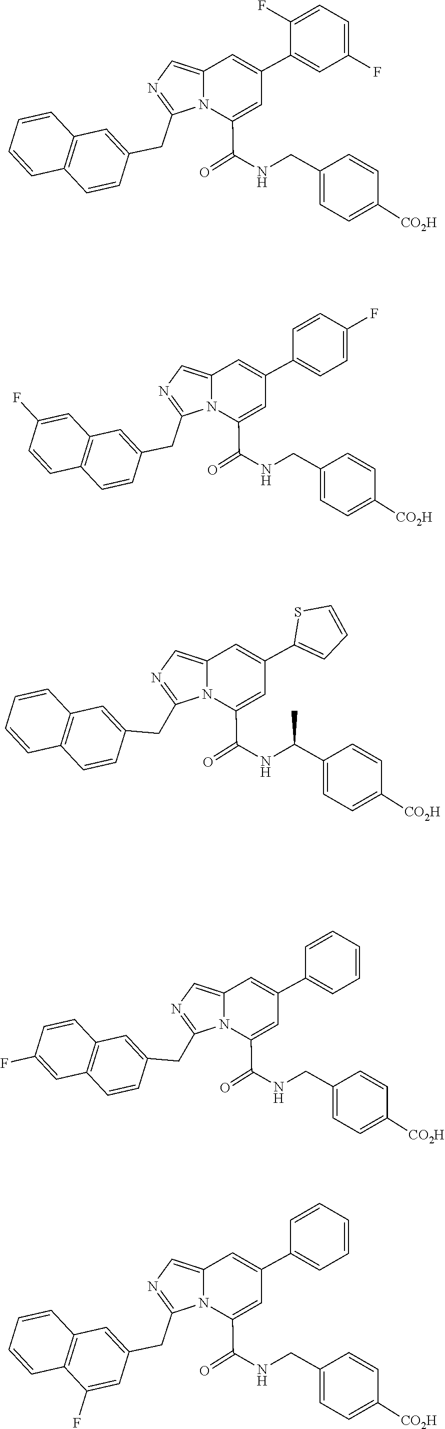 [SMILES: O=C(O)c1ccc(CNC(=O)c2cc(-c3cc(F)ccc3F)cc3cnc(Cc4ccc5ccccc5c4)n23)cc1 ; O=C(O)c1ccc(CNC(=O)c2cc(-c3ccc(F)cc3)cc3cnc(Cc4ccc5ccc(F)cc5c4)n23)cc1 ; C[C@H](NC(=O)c1cc(-c2cccs2)cc2cnc(Cc3ccc4ccccc4c3)n12)c1ccc(C(=O)O)cc1 ; O=C(O)c1ccc(CNC(=O)c2cc(-c3ccccc3)cc3cnc(Cc4ccc5cc(F)ccc5c4)n23)cc1 ; O=C(O)c1ccc(CNC(=O)c2cc(-c3ccccc3)cc3cnc(Cc4cc(F)c5ccccc5c4)n23)cc1]

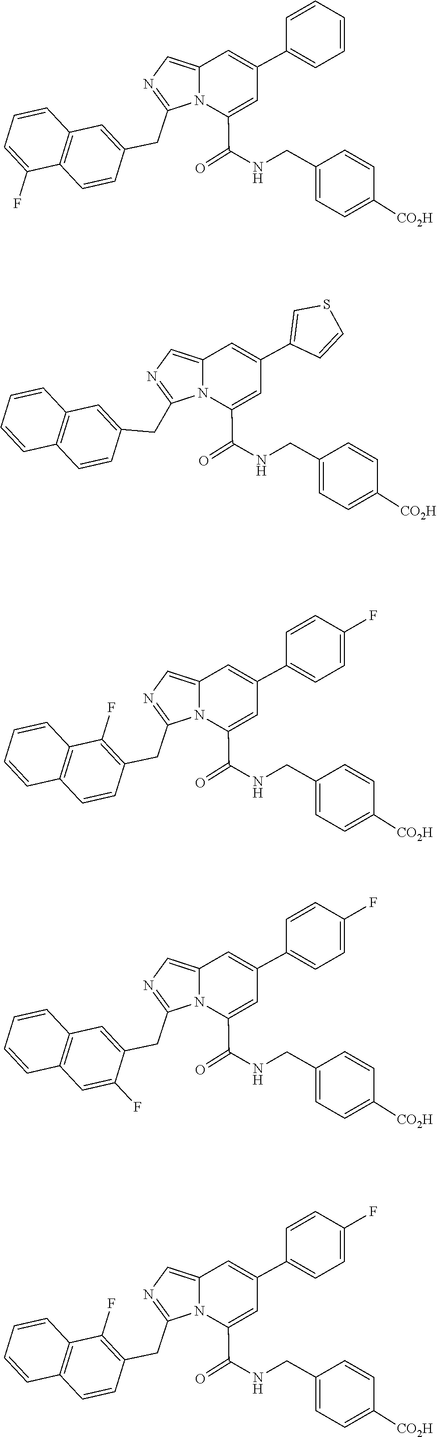 [SMILES: O=C(O)c1ccc(CNC(=O)c2cc(-c3ccccc3)cc3cnc(Cc4ccc5c(F)cccc5c4)n23)cc1 ; O=C(O)c1ccc(CNC(=O)c2cc(-c3ccsc3)cc3cnc(Cc4ccc5ccccc5c4)n23)cc1 ; O=C(O)c1ccc(CNC(=O)c2cc(-c3ccc(F)cc3)cc3cnc(Cc4ccc5ccccc5c4F)n23)cc1 ; O=C(O)c1ccc(CNC(=O)c2cc(-c3ccc(F)cc3)cc3cnc(Cc4cc5ccccc5cc4F)n23)cc1 ; O=C(O)c1ccc(CNC(=O)c2cc(-c3ccc(F)cc3)cc3cnc(Cc4ccc5ccccc5c4F)n23)cc1]

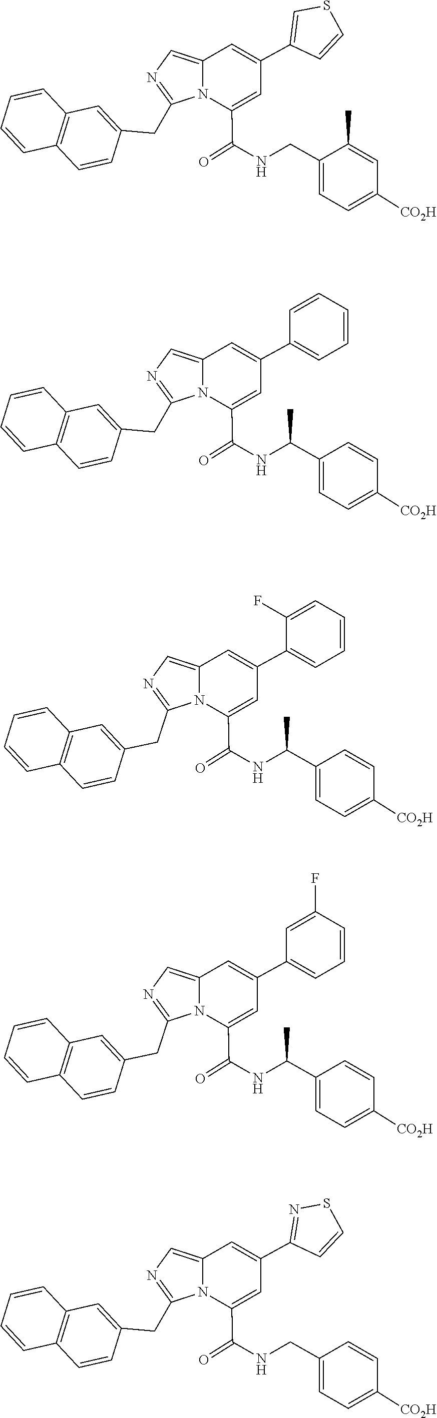

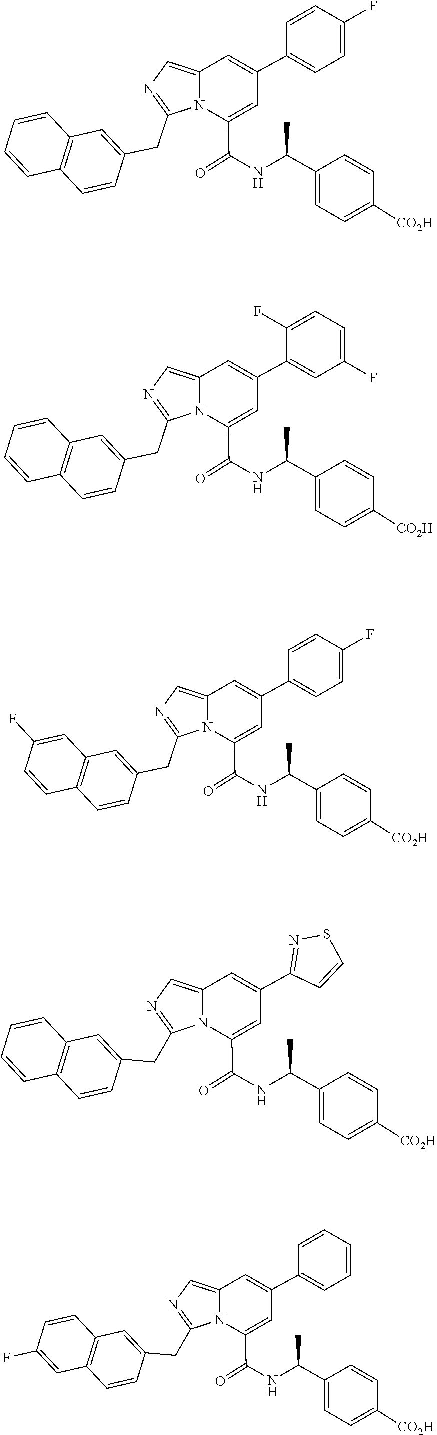 [SMILES: C[C@H](NC(=O)c1cc(-c2ccc(F)cc2)cc2cnc(Cc3ccc4ccccc4c3)n12)c1ccc(C(=O)O)cc1 ; C[C@H](NC(=O)c1cc(-c2cc(F)ccc2F)cc2cnc(Cc3ccc4ccccc4c3)n12)c1ccc(C(=O)O)cc1 ; C[C@H](NC(=O)c1cc(-c2ccc(F)cc2)cc2cnc(Cc3ccc4ccc(F)cc4c3)n12)c1ccc(C(=O)O)cc1 ; C[C@H](NC(=O)c1cc(-c2ccsn2)cc2cnc(Cc3ccc4ccccc4c3)n12)c1ccc(C(=O)O)cc1 ; C[C@H](NC(=O)c1cc(-c2ccccc2)cc2cnc(Cc3ccc4cc(F)ccc4c3)n12)c1ccc(C(=O)O)cc1]

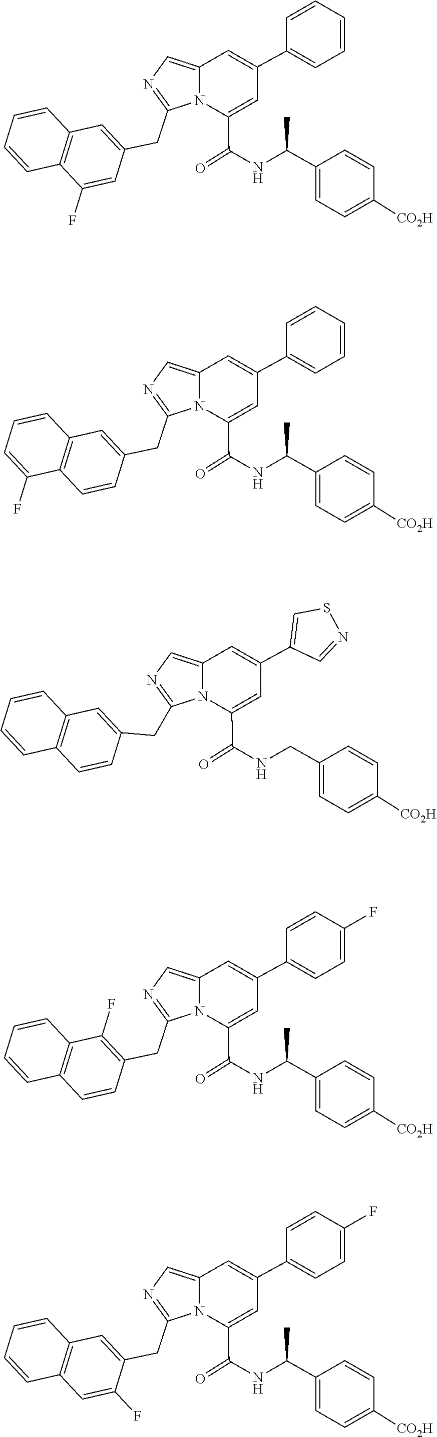 [SMILES: C[C@H](NC(=O)c1cc(-c2ccccc2)cc2cnc(Cc3cc(F)c4ccccc4c3)n12)c1ccc(C(=O)O)cc1 ; C[C@H](NC(=O)c1cc(-c2ccccc2)cc2cnc(Cc3ccc4c(F)cccc4c3)n12)c1ccc(C(=O)O)cc1 ; O=C(O)c1ccc(CNC(=O)c2cc(-c3cnsc3)cc3cnc(Cc4ccc5ccccc5c4)n23)cc1 ; C[C@H](NC(=O)c1cc(-c2ccc(F)cc2)cc2cnc(Cc3ccc4ccccc4c3F)n12)c1ccc(C(=O)O)cc1 ; C[C@H](NC(=O)c1cc(-c2ccc(F)cc2)cc2cnc(Cc3cc4ccccc4cc3F)n12)c1ccc(C(=O)O)cc1]

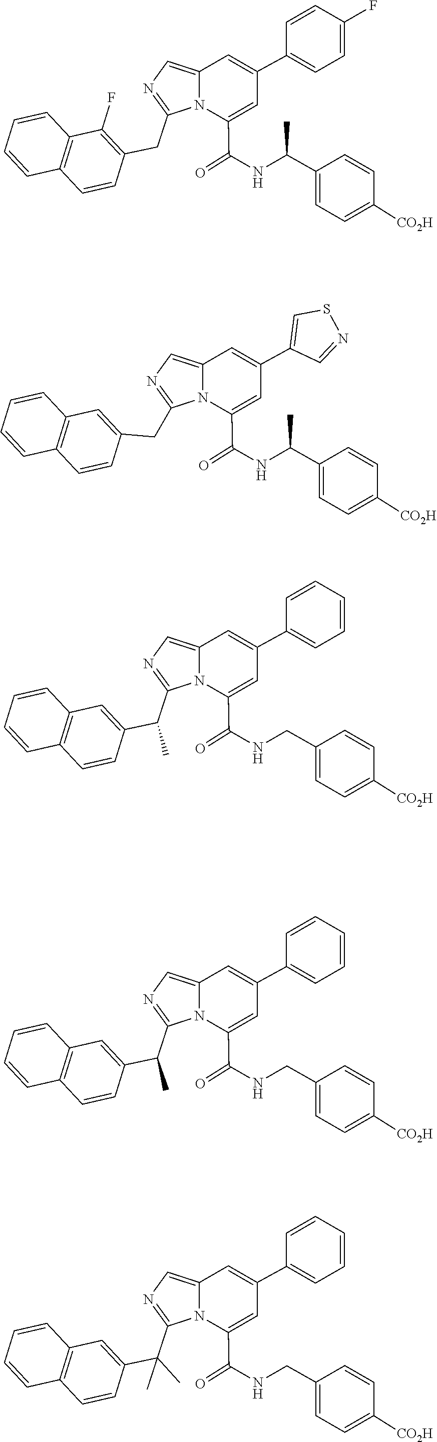 [SMILES: C[C@H](NC(=O)c1cc(-c2ccc(F)cc2)cc2cnc(Cc3ccc4ccccc4c3F)n12)c1ccc(C(=O)O)cc1 ; C[C@H](NC(=O)c1cc(-c2cnsc2)cc2cnc(Cc3ccc4ccccc4c3)n12)c1ccc(C(=O)O)cc1 ; C[C@H](c1ccc2ccccc2c1)c1ncc2cc(-c3ccccc3)cc(C(=O)NCc3ccc(C(=O)O)cc3)n12 ; C[C@@H](c1ccc2ccccc2c1)c1ncc2cc(-c3ccccc3)cc(C(=O)NCc3ccc(C(=O)O)cc3)n12 ; CC(C)(c1ccc2ccccc2c1)c1ncc2cc(-c3ccccc3)cc(C(=O)NCc3ccc(C(=O)O)cc3)n12]

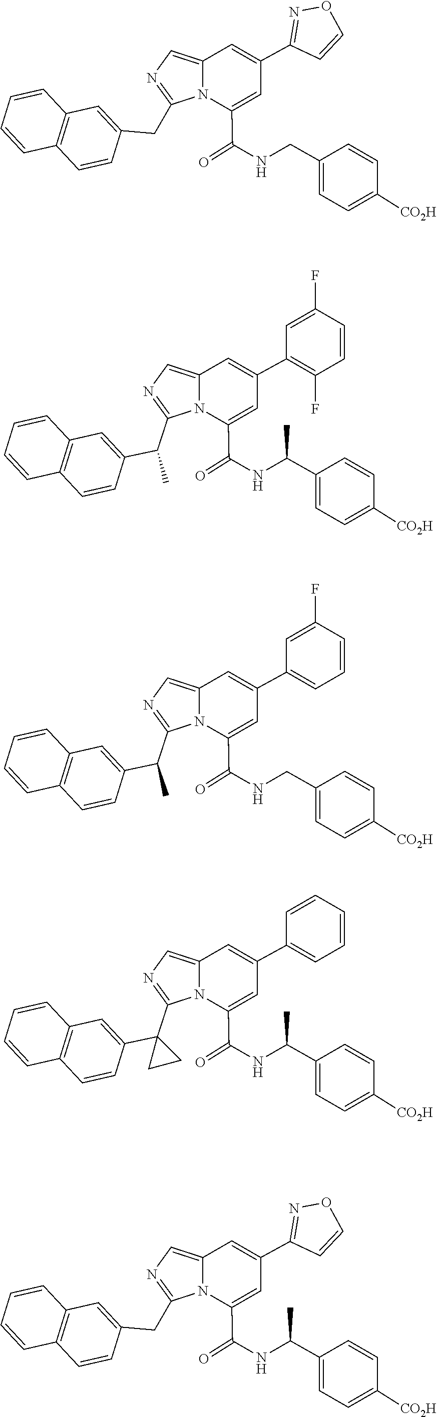 [SMILES: O=C(O)c1ccc(CNC(=O)c2cc(-c3ccon3)cc3cnc(Cc4ccc5ccccc5c4)n23)cc1 ; C[C@H](c1ccc2ccccc2c1)c1ncc2cc(-c3cc(F)ccc3F)cc(C(=O)N[C@@H](C)c3ccc(C(=O)O)cc3)n12 ; C[C@@H](c1ccc2ccccc2c1)c1ncc2cc(-c3cccc(F)c3)cc(C(=O)NCc3ccc(C(=O)O)cc3)n12 ; C[C@H](NC(=O)c1cc(-c2ccccc2)cc2cnc(C3(c4ccc5ccccc5c4)CC3)n12)c1ccc(C(=O)O)cc1 ; C[C@H](NC(=O)c1cc(-c2ccon2)cc2cnc(Cc3ccc4ccccc4c3)n12)c1ccc(C(=O)O)cc1]

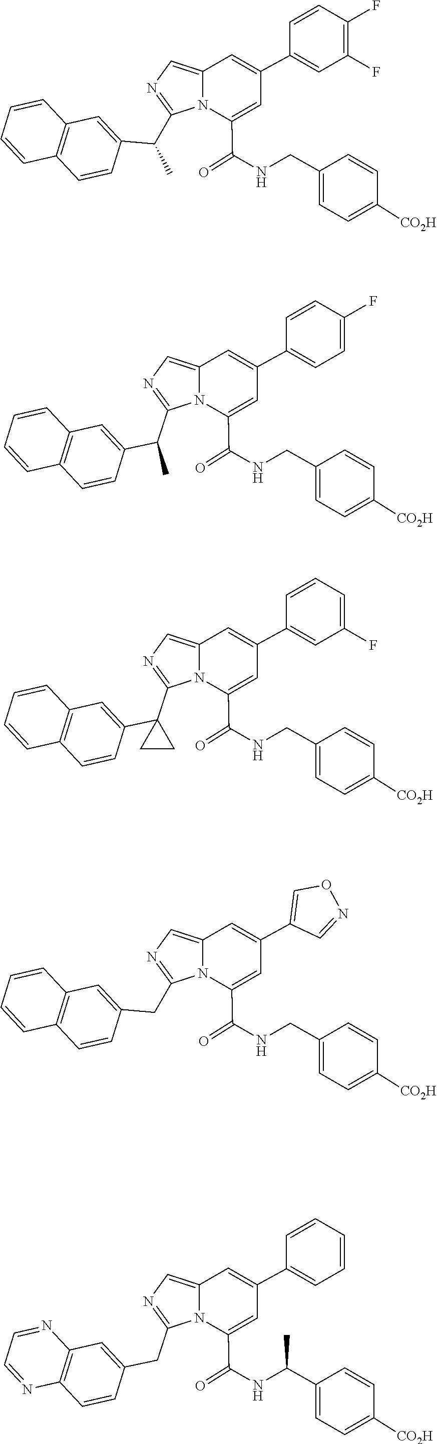 [SMILES: C[C@H](c1ccc2ccccc2c1)c1ncc2cc(-c3ccc(F)c(F)c3)cc(C(=O)NCc3ccc(C(=O)O)cc3)n12 ; C[C@@H](c1ccc2ccccc2c1)c1ncc2cc(-c3ccc(F)cc3)cc(C(=O)NCc3ccc(C(=O)O)cc3)n12 ; O=C(O)c1ccc(CNC(=O)c2cc(-c3cccc(F)c3)cc3cnc(C4(c5ccc6ccccc6c5)CC4)n23)cc1 ; O=C(O)c1ccc(CNC(=O)c2cc(-c3cnoc3)cc3cnc(Cc4ccc5ccccc5c4)n23)cc1 ; C[C@H](NC(=O)c1cc(-c2ccccc2)cc2cnc(Cc3ccc4nccnc4c3)n12)c1ccc(C(=O)O)cc1]

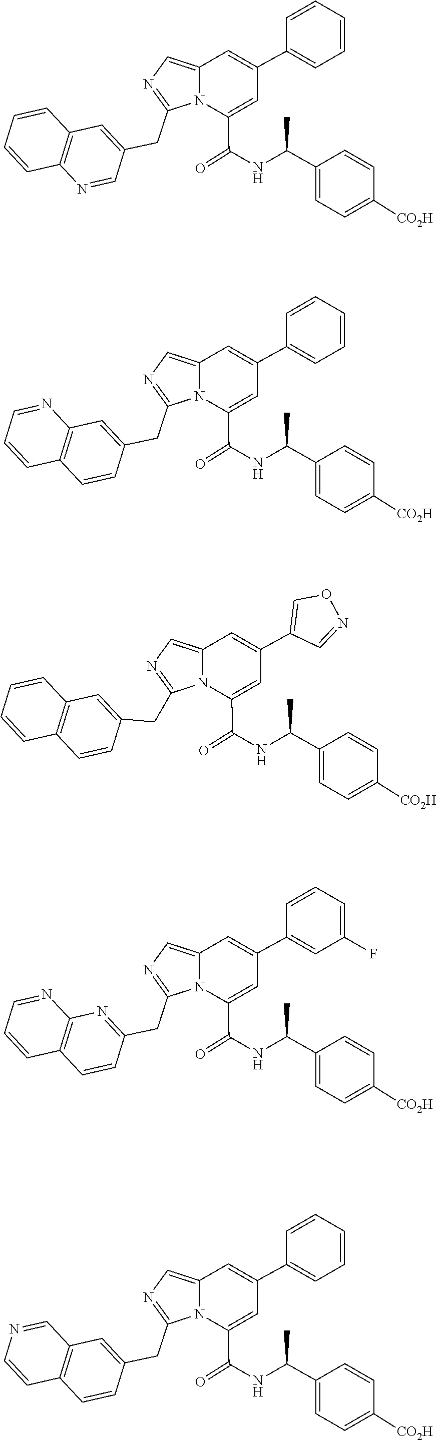 [SMILES: C[C@H](NC(=O)c1cc(-c2ccccc2)cc2cnc(Cc3cnc4ccccc4c3)n12)c1ccc(C(=O)O)cc1 ; C[C@H](NC(=O)c1cc(-c2ccccc2)cc2cnc(Cc3ccc4cccnc4c3)n12)c1ccc(C(=O)O)cc1 ; C[C@H](NC(=O)c1cc(-c2cnoc2)cc2cnc(Cc3ccc4ccccc4c3)n12)c1ccc(C(=O)O)cc1 ; C[C@H](NC(=O)c1cc(-c2cccc(F)c2)cc2cnc(Cc3ccc4cccnc4n3)n12)c1ccc(C(=O)O)cc1 ; C[C@H](NC(=O)c1cc(-c2ccccc2)cc2cnc(Cc3ccc4ccncc4c3)n12)c1ccc(C(=O)O)cc1]

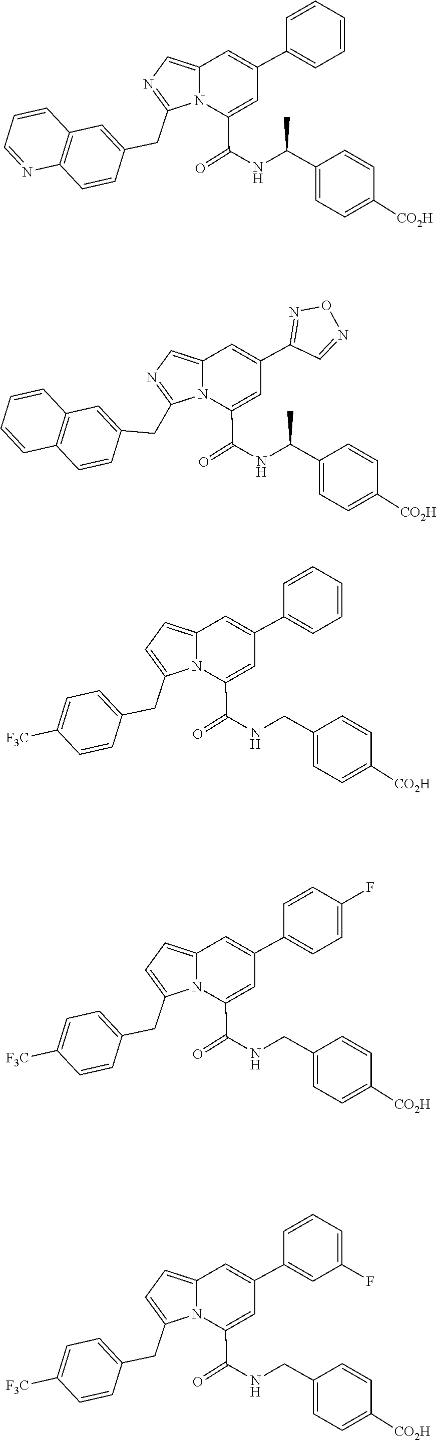 [SMILES: C[C@H](NC(=O)c1cc(-c2ccccc2)cc2cnc(Cc3ccc4ncccc4c3)n12)c1ccc(C(=O)O)cc1 ; C[C@H](NC(=O)c1cc(-c2cnon2)cc2cnc(Cc3ccc4ccccc4c3)n12)c1ccc(C(=O)O)cc1 ; O=C(O)c1ccc(CNC(=O)c2cc(-c3ccccc3)cc3ccc(Cc4ccc(C(F)(F)F)cc4)n23)cc1 ; O=C(O)c1ccc(CNC(=O)c2cc(-c3ccc(F)cc3)cc3ccc(Cc4ccc(C(F)(F)F)cc4)n23)cc1 ; O=C(O)c1ccc(CNC(=O)c2cc(-c3cccc(F)c3)cc3ccc(Cc4ccc(C(F)(F)F)cc4)n23)cc1]

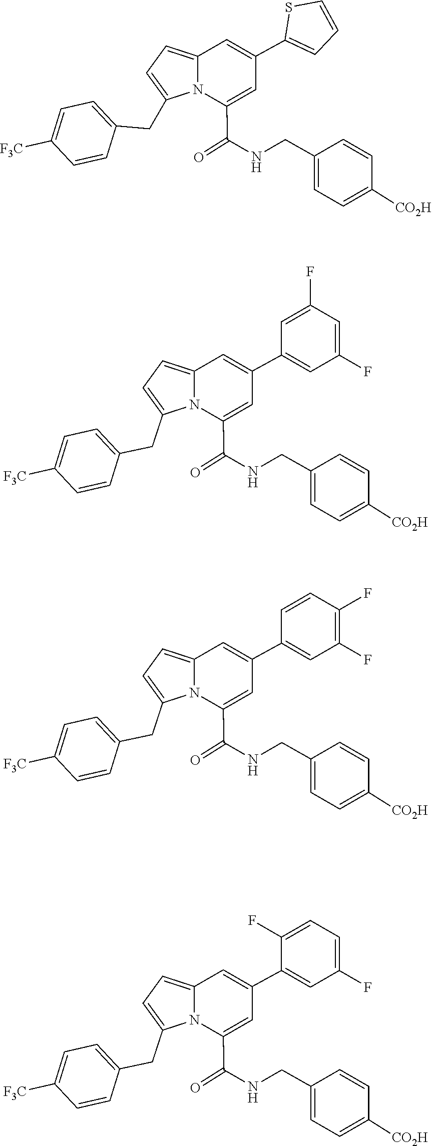 [SMILES: O=C(O)c1ccc(CNC(=O)c2cc(-c3cccs3)cc3ccc(Cc4ccc(C(F)(F)F)cc4)n23)cc1 ; O=C(O)c1ccc(CNC(=O)c2cc(-c3cc(F)cc(F)c3)cc3ccc(Cc4ccc(C(F)(F)F)cc4)n23)cc1 ; O=C(O)c1ccc(CNC(=O)c2cc(-c3ccc(F)c(F)c3)cc3ccc(Cc4ccc(C(F)(F)F)cc4)n23)cc1 ; O=C(O)c1ccc(CNC(=O)c2cc(-c3cc(F)ccc3F)cc3ccc(Cc4ccc(C(F)(F)F)cc4)n23)cc1]

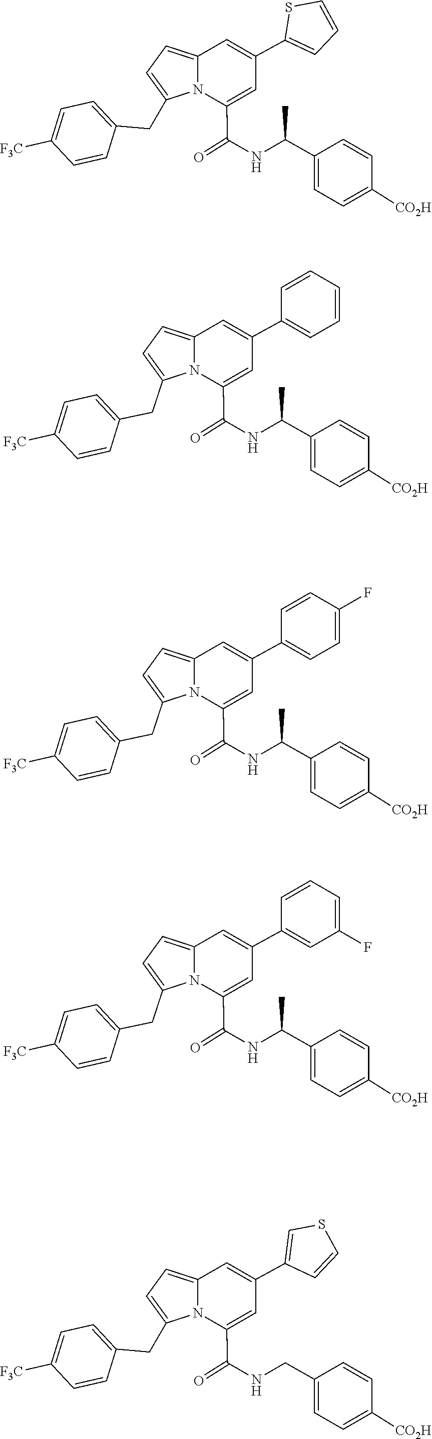 [SMILES: C[C@H](NC(=O)c1cc(-c2cccs2)cc2ccc(Cc3ccc(C(F)(F)F)cc3)n12)c1ccc(C(=O)O)cc1 ; C[C@H](NC(=O)c1cc(-c2ccccc2)cc2ccc(Cc3ccc(C(F)(F)F)cc3)n12)c1ccc(C(=O)O)cc1 ; C[C@H](NC(=O)c1cc(-c2ccc(F)cc2)cc2ccc(Cc3ccc(C(F)(F)F)cc3)n12)c1ccc(C(=O)O)cc1 ; C[C@H](NC(=O)c1cc(-c2cccc(F)c2)cc2ccc(Cc3ccc(C(F)(F)F)cc3)n12)c1ccc(C(=O)O)cc1 ; O=C(O)c1ccc(CNC(=O)c2cc(-c3ccsc3)cc3ccc(Cc4ccc(C(F)(F)F)cc4)n23)cc1]

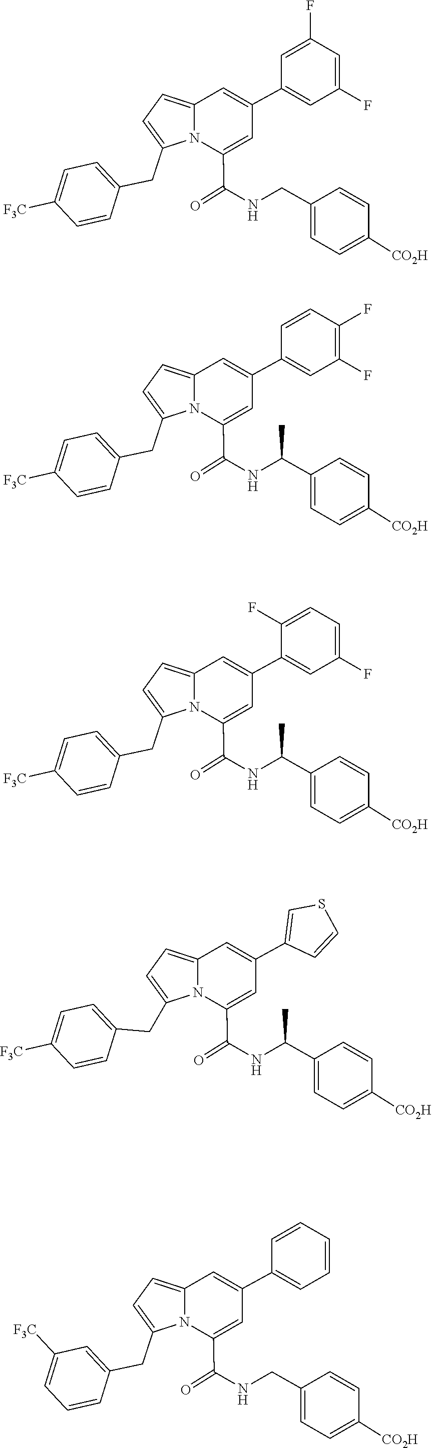 [SMILES: O=C(O)c1ccc(CNC(=O)c2cc(-c3cc(F)cc(F)c3)cc3ccc(Cc4ccc(C(F)(F)F)cc4)n23)cc1 ; C[C@H](NC(=O)c1cc(-c2ccc(F)c(F)c2)cc2ccc(Cc3ccc(C(F)(F)F)cc3)n12)c1ccc(C(=O)O)cc1 ; C[C@H](NC(=O)c1cc(-c2cc(F)ccc2F)cc2ccc(Cc3ccc(C(F)(F)F)cc3)n12)c1ccc(C(=O)O)cc1 ; C[C@H](NC(=O)c1cc(-c2ccsc2)cc2ccc(Cc3ccc(C(F)(F)F)cc3)n12)c1ccc(C(=O)O)cc1 ; O=C(O)c1ccc(CNC(=O)c2cc(-c3ccccc3)cc3ccc(Cc4cccc(C(F)(F)F)c4)n23)cc1]

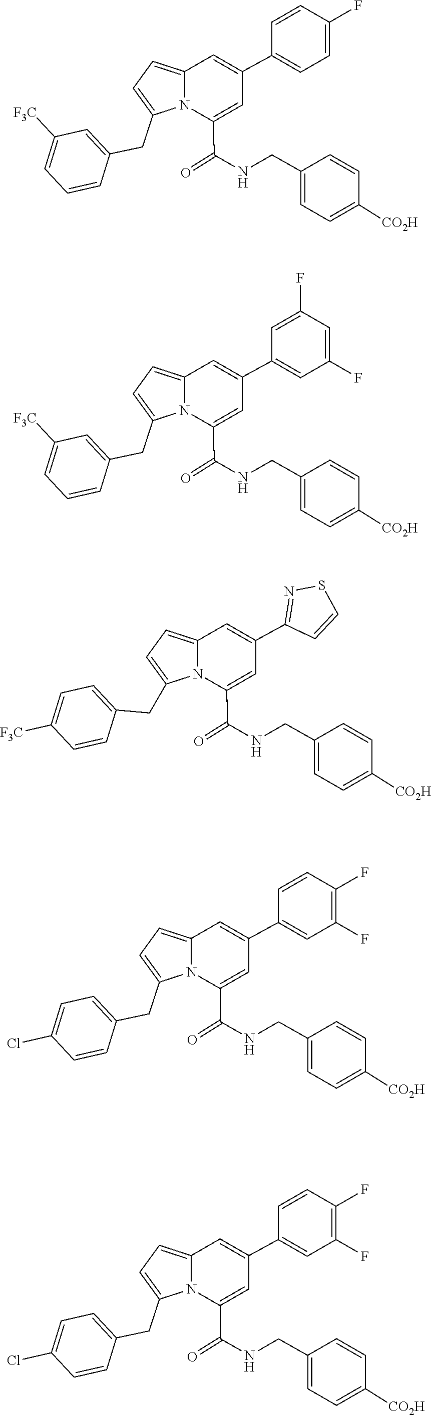 [SMILES: O=C(O)c1ccc(CNC(=O)c2cc(-c3ccc(F)cc3)cc3ccc(Cc4cccc(C(F)(F)F)c4)n23)cc1 ; O=C(O)c1ccc(CNC(=O)c2cc(-c3cc(F)cc(F)c3)cc3ccc(Cc4cccc(C(F)(F)F)c4)n23)cc1 ; O=C(O)c1ccc(CNC(=O)c2cc(-c3ccsn3)cc3ccc(Cc4ccc(C(F)(F)F)cc4)n23)cc1 ; O=C(O)c1ccc(CNC(=O)c2cc(-c3ccc(F)c(F)c3)cc3ccc(Cc4ccc(Cl)cc4)n23)cc1 ; O=C(O)c1ccc(CNC(=O)c2cc(-c3ccc(F)c(F)c3)cc3ccc(Cc4ccc(Cl)cc4)n23)cc1]

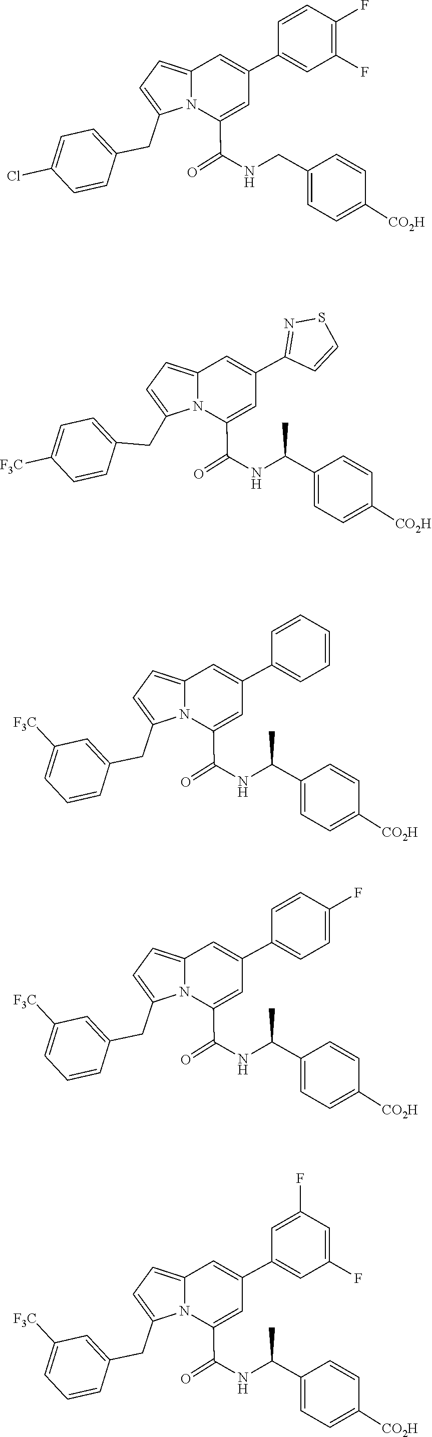 [SMILES: O=C(O)c1ccc(CNC(=O)c2cc(-c3ccc(F)c(F)c3)cc3ccc(Cc4ccc(Cl)cc4)n23)cc1 ; C[C@H](NC(=O)c1cc(-c2ccsn2)cc2ccc(Cc3ccc(C(F)(F)F)cc3)n12)c1ccc(C(=O)O)cc1 ; C[C@H](NC(=O)c1cc(-c2ccccc2)cc2ccc(Cc3cccc(C(F)(F)F)c3)n12)c1ccc(C(=O)O)cc1 ; C[C@H](NC(=O)c1cc(-c2ccc(F)cc2)cc2ccc(Cc3cccc(C(F)(F)F)c3)n12)c1ccc(C(=O)O)cc1 ; C[C@H](NC(=O)c1cc(-c2cc(F)cc(F)c2)cc2ccc(Cc3cccc(C(F)(F)F)c3)n12)c1ccc(C(=O)O)cc1]

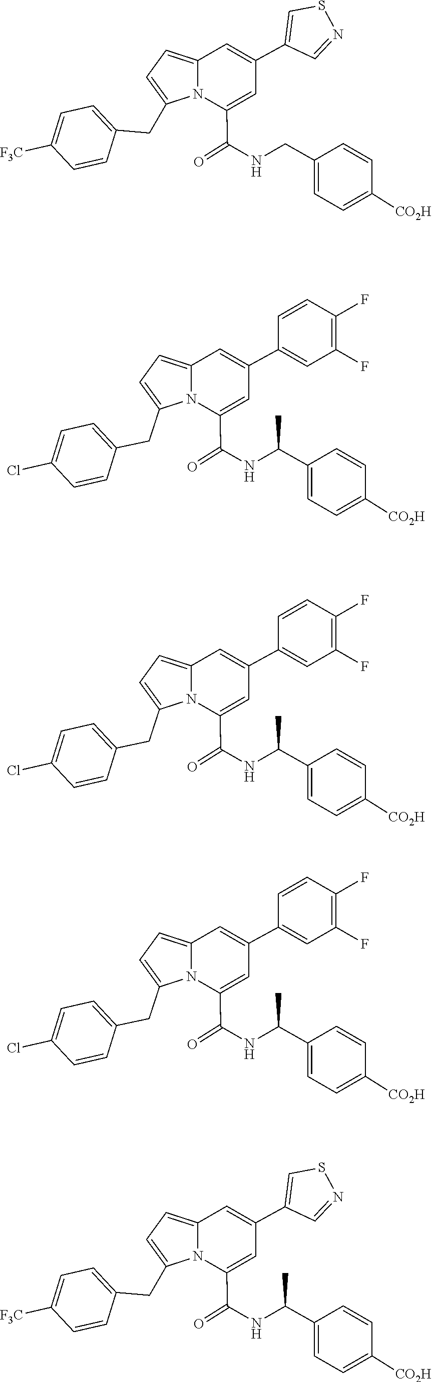 [SMILES: O=C(O)c1ccc(CNC(=O)c2cc(-c3cnsc3)cc3ccc(Cc4ccc(C(F)(F)F)cc4)n23)cc1 ; C[C@H](NC(=O)c1cc(-c2ccc(F)c(F)c2)cc2ccc(Cc3ccc(Cl)cc3)n12)c1ccc(C(=O)O)cc1 ; C[C@H](NC(=O)c1cc(-c2ccc(F)c(F)c2)cc2ccc(Cc3ccc(Cl)cc3)n12)c1ccc(C(=O)O)cc1 ; C[C@H](NC(=O)c1cc(-c2ccc(F)c(F)c2)cc2ccc(Cc3ccc(Cl)cc3)n12)c1ccc(C(=O)O)cc1 ; C[C@H](NC(=O)c1cc(-c2cnsc2)cc2ccc(Cc3ccc(C(F)(F)F)cc3)n12)c1ccc(C(=O)O)cc1]

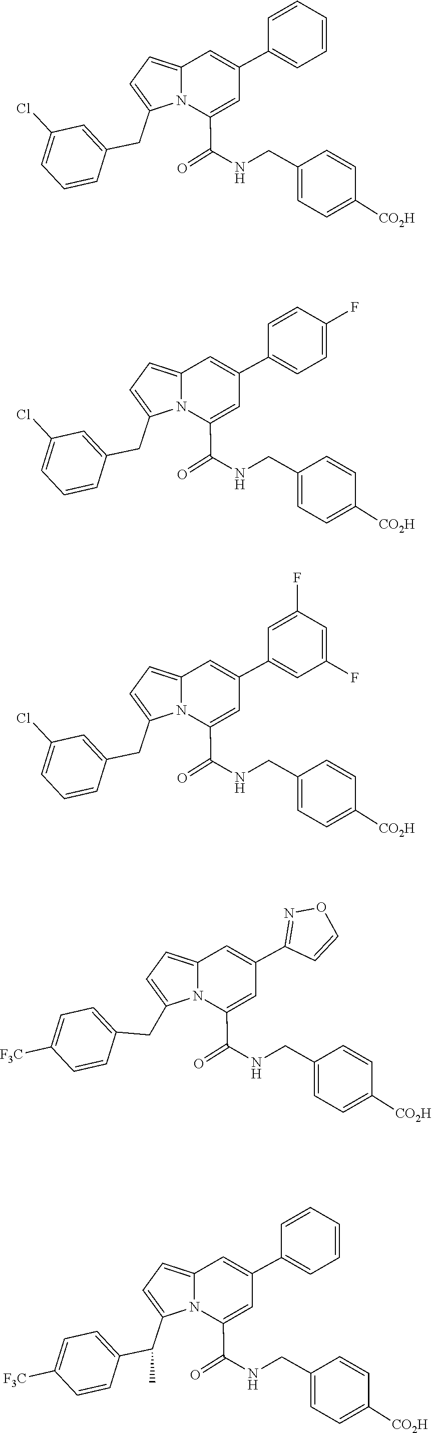 [SMILES: O=C(O)c1ccc(CNC(=O)c2cc(-c3ccccc3)cc3ccc(Cc4cccc(Cl)c4)n23)cc1 ; O=C(O)c1ccc(CNC(=O)c2cc(-c3ccc(F)cc3)cc3ccc(Cc4cccc(Cl)c4)n23)cc1 ; O=C(O)c1ccc(CNC(=O)c2cc(-c3cc(F)cc(F)c3)cc3ccc(Cc4cccc(Cl)c4)n23)cc1 ; O=C(O)c1ccc(CNC(=O)c2cc(-c3ccon3)cc3ccc(Cc4ccc(C(F)(F)F)cc4)n23)cc1 ; C[C@H](c1ccc(C(F)(F)F)cc1)c1ccc2cc(-c3ccccc3)cc(C(=O)NCc3ccc(C(=O)O)cc3)n12]

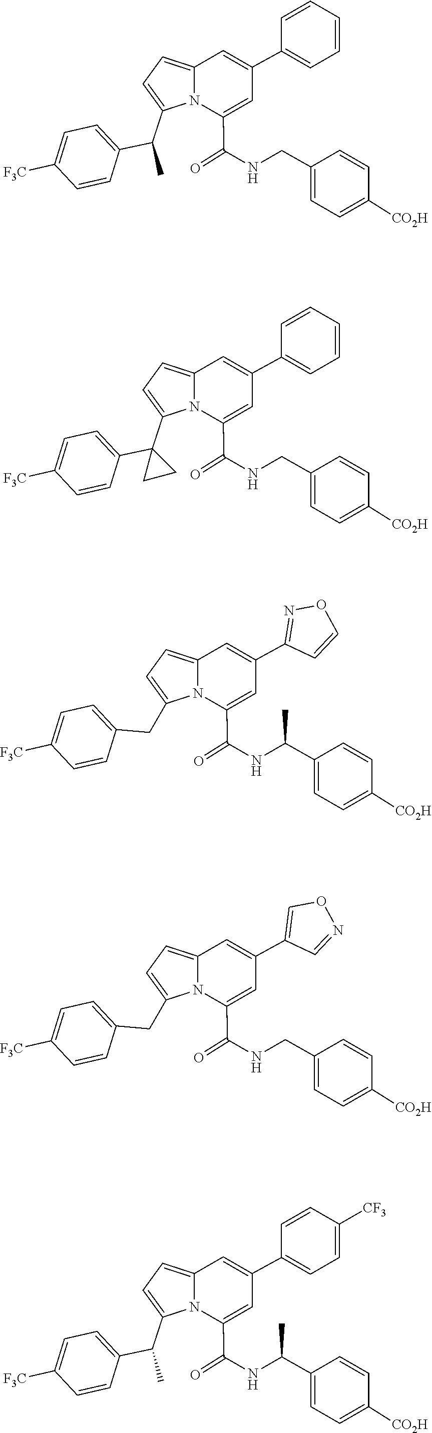 [SMILES: C[C@@H](c1ccc(C(F)(F)F)cc1)c1ccc2cc(-c3ccccc3)cc(C(=O)NCc3ccc(C(=O)O)cc3)n12 ; O=C(O)c1ccc(CNC(=O)c2cc(-c3ccccc3)cc3ccc(C4(c5ccc(C(F)(F)F)cc5)CC4)n23)cc1 ; C[C@H](NC(=O)c1cc(-c2ccon2)cc2ccc(Cc3ccc(C(F)(F)F)cc3)n12)c1ccc(C(=O)O)cc1 ; O=C(O)c1ccc(CNC(=O)c2cc(-c3cnoc3)cc3ccc(Cc4ccc(C(F)(F)F)cc4)n23)cc1 ; C[C@H](c1ccc(C(F)(F)F)cc1)c1ccc2cc(-c3ccc(C(F)(F)F)cc3)cc(C(=O)N[C@@H](C)c3ccc(C(=O)O)cc3)n12]

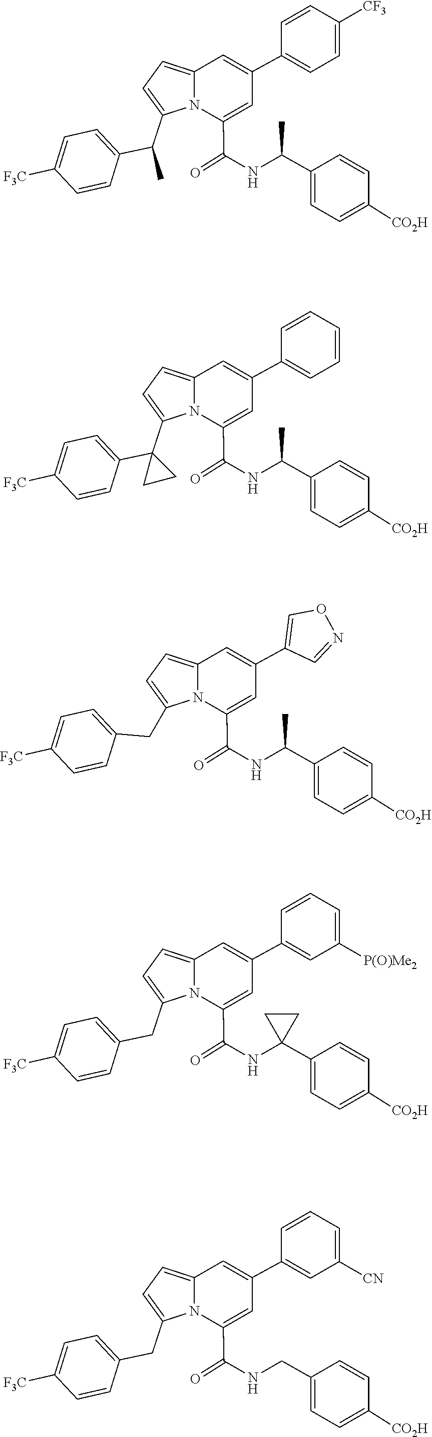

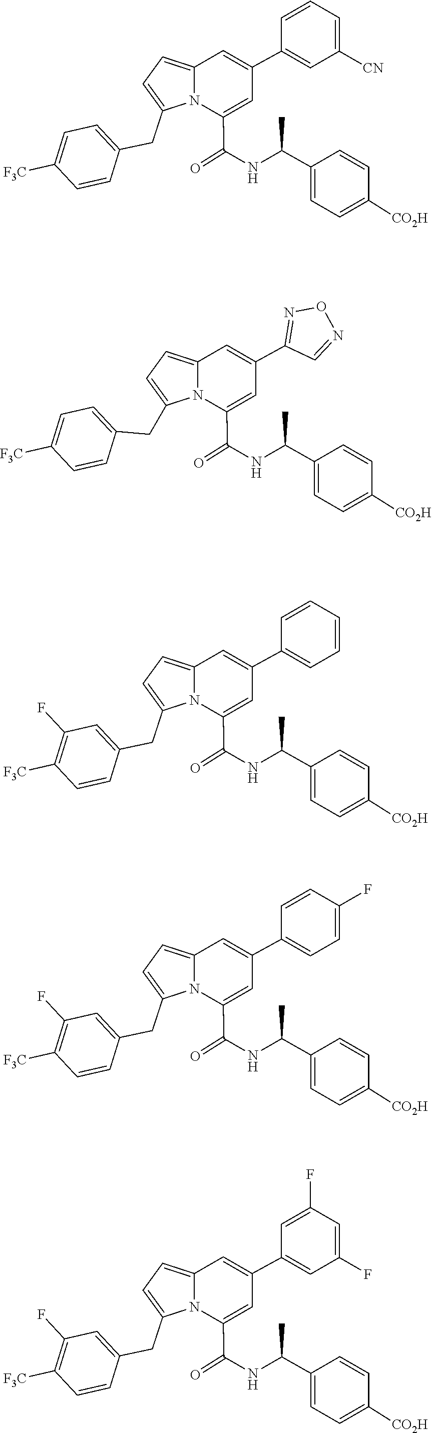 [SMILES: C[C@H](NC(=O)c1cc(-c2cccc(C#N)c2)cc2ccc(Cc3ccc(C(F)(F)F)cc3)n12)c1ccc(C(=O)O)cc1 ; C[C@H](NC(=O)c1cc(-c2cnon2)cc2ccc(Cc3ccc(C(F)(F)F)cc3)n12)c1ccc(C(=O)O)cc1 ; C[C@H](NC(=O)c1cc(-c2ccccc2)cc2ccc(Cc3ccc(C(F)(F)F)c(F)c3)n12)c1ccc(C(=O)O)cc1 ; C[C@H](NC(=O)c1cc(-c2ccc(F)cc2)cc2ccc(Cc3ccc(C(F)(F)F)c(F)c3)n12)c1ccc(C(=O)O)cc1 ; C[C@H](NC(=O)c1cc(-c2cc(F)cc(F)c2)cc2ccc(Cc3ccc(C(F)(F)F)c(F)c3)n12)c1ccc(C(=O)O)cc1]

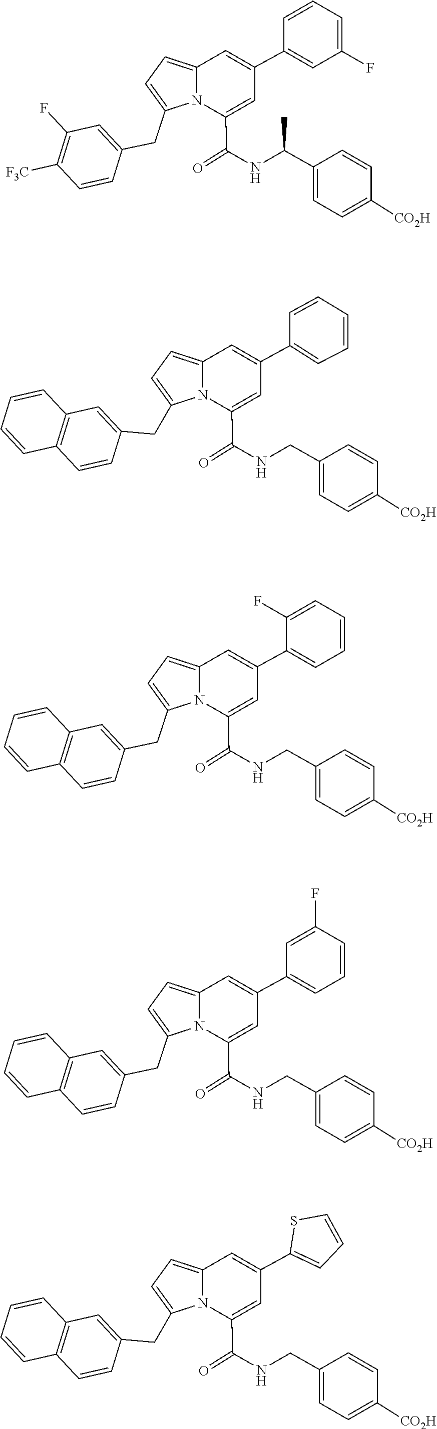 [SMILES: C[C@H](NC(=O)c1cc(-c2cccc(F)c2)cc2ccc(Cc3ccc(C(F)(F)F)c(F)c3)n12)c1ccc(C(=O)O)cc1 ; O=C(O)c1ccc(CNC(=O)c2cc(-c3ccccc3)cc3ccc(Cc4ccc5ccccc5c4)n23)cc1 ; O=C(O)c1ccc(CNC(=O)c2cc(-c3ccccc3F)cc3ccc(Cc4ccc5ccccc5c4)n23)cc1 ; O=C(O)c1ccc(CNC(=O)c2cc(-c3cccc(F)c3)cc3ccc(Cc4ccc5ccccc5c4)n23)cc1 ; O=C(O)c1ccc(CNC(=O)c2cc(-c3cccs3)cc3ccc(Cc4ccc5ccccc5c4)n23)cc1]

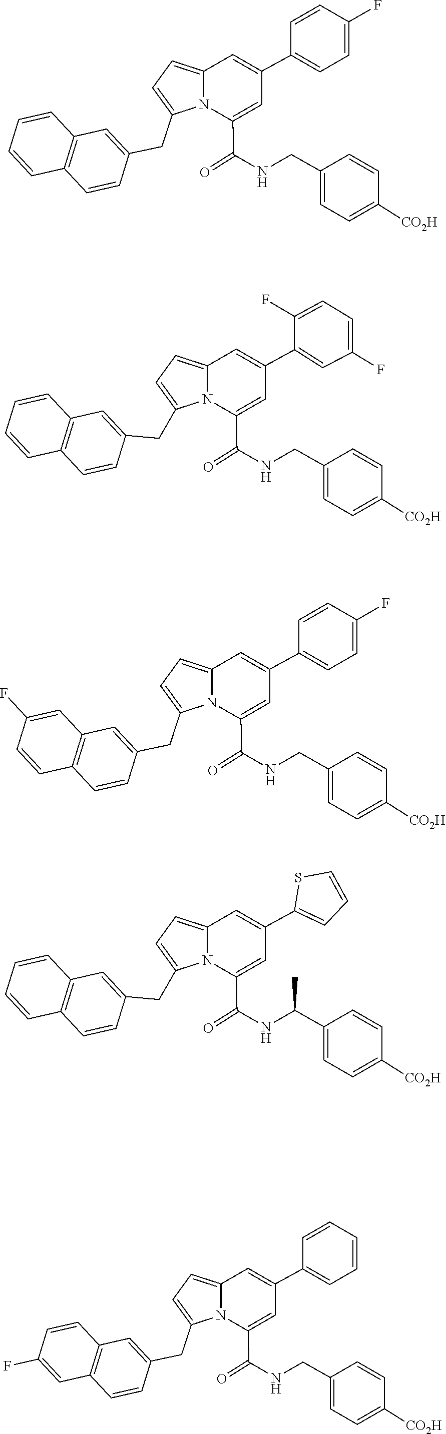 [SMILES: O=C(O)c1ccc(CNC(=O)c2cc(-c3ccc(F)cc3)cc3ccc(Cc4ccc5ccccc5c4)n23)cc1 ; O=C(O)c1ccc(CNC(=O)c2cc(-c3cc(F)ccc3F)cc3ccc(Cc4ccc5ccccc5c4)n23)cc1 ; O=C(O)c1ccc(CNC(=O)c2cc(-c3ccc(F)cc3)cc3ccc(Cc4ccc5ccc(F)cc5c4)n23)cc1 ; C[C@H](NC(=O)c1cc(-c2cccs2)cc2ccc(Cc3ccc4ccccc4c3)n12)c1ccc(C(=O)O)cc1 ; O=C(O)c1ccc(CNC(=O)c2cc(-c3ccccc3)cc3ccc(Cc4ccc5cc(F)ccc5c4)n23)cc1]

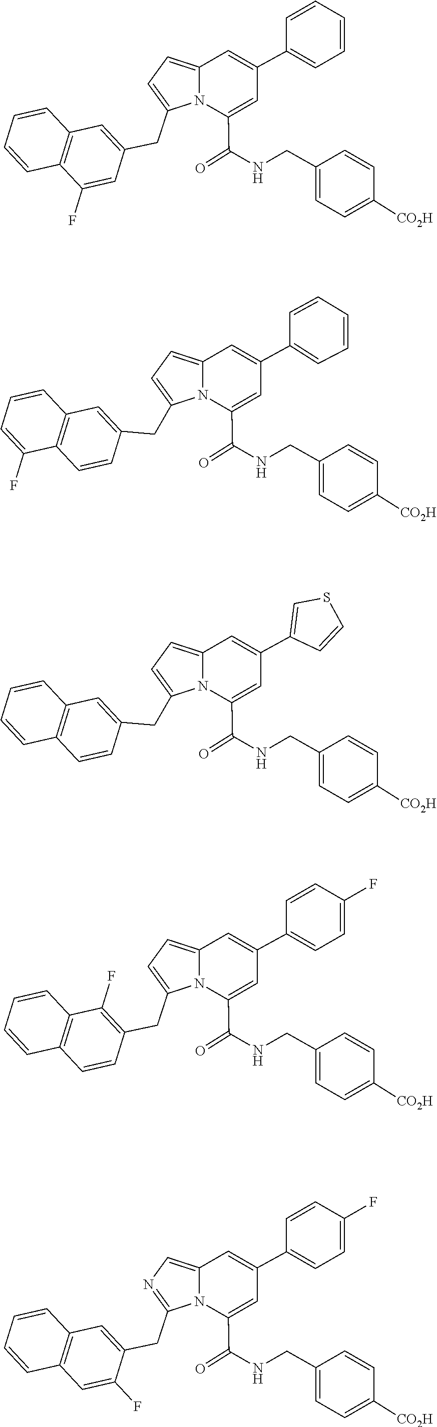 [SMILES: O=C(O)c1ccc(CNC(=O)c2cc(-c3ccccc3)cc3ccc(Cc4cc(F)c5ccccc5c4)n23)cc1 ; O=C(O)c1ccc(CNC(=O)c2cc(-c3ccccc3)cc3ccc(Cc4ccc5c(F)cccc5c4)n23)cc1 ; O=C(O)c1ccc(CNC(=O)c2cc(-c3ccsc3)cc3ccc(Cc4ccc5ccccc5c4)n23)cc1 ; O=C(O)c1ccc(CNC(=O)c2cc(-c3ccc(F)cc3)cc3ccc(Cc4ccc5ccccc5c4F)n23)cc1 ; O=C(O)c1ccc(CNC(=O)c2cc(-c3ccc(F)cc3)cc3cnc(Cc4cc5ccccc5cc4F)n23)cc1]

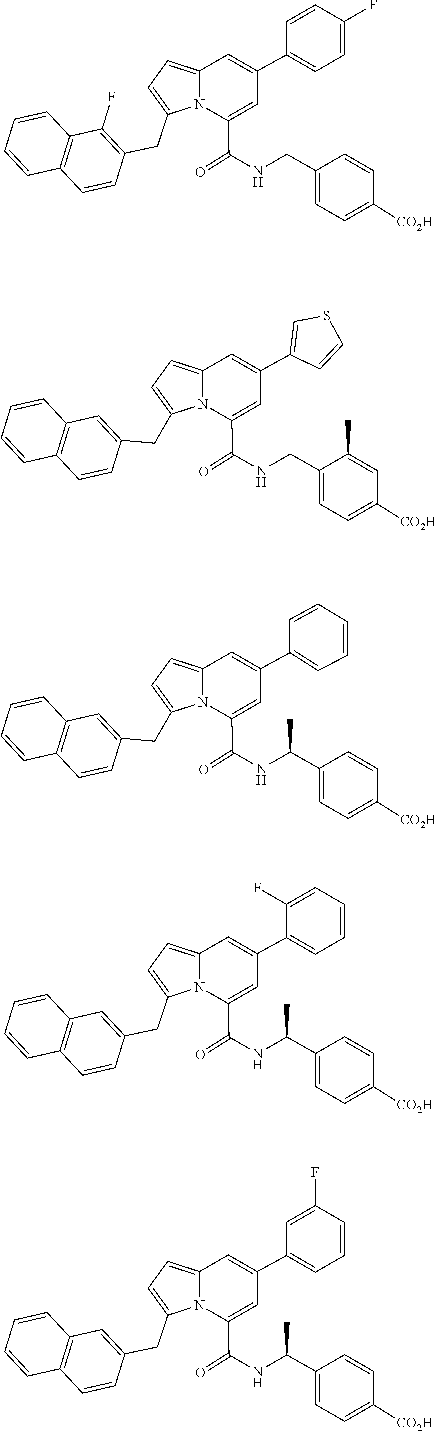

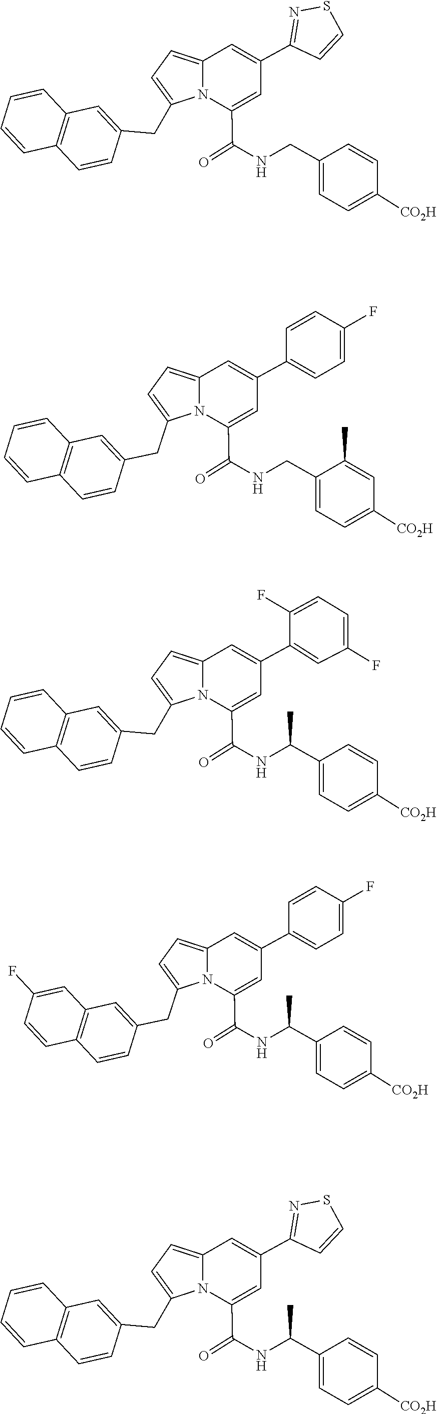

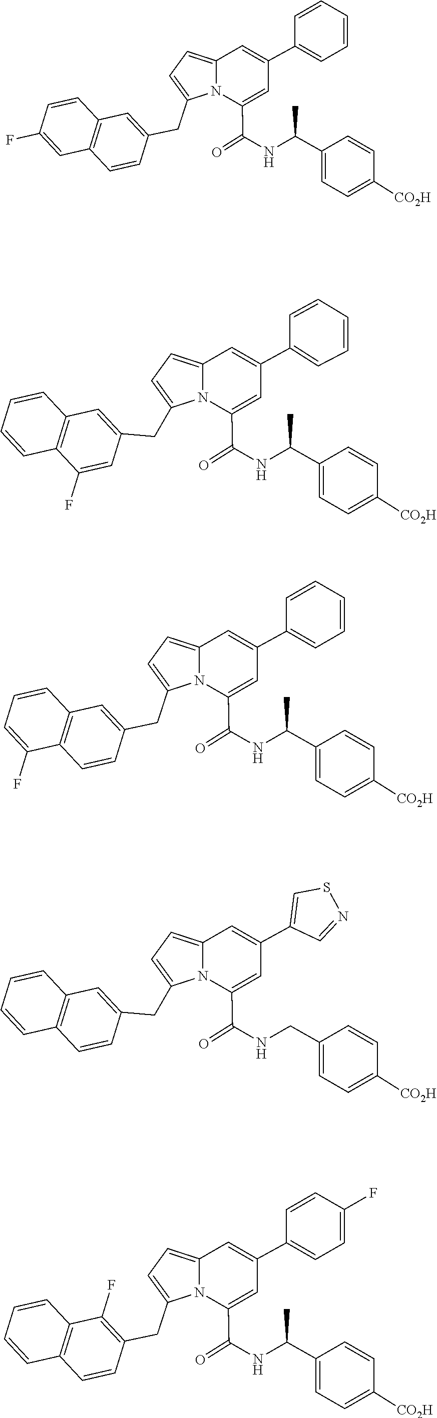 [SMILES: C[C@H](NC(=O)c1cc(-c2ccccc2)cc2ccc(Cc3ccc4cc(F)ccc4c3)n12)c1ccc(C(=O)O)cc1 ; C[C@H](NC(=O)c1cc(-c2ccccc2)cc2ccc(Cc3cc(F)c4ccccc4c3)n12)c1ccc(C(=O)O)cc1 ; C[C@H](NC(=O)c1cc(-c2ccccc2)cc2ccc(Cc3ccc4c(F)cccc4c3)n12)c1ccc(C(=O)O)cc1 ; O=C(O)c1ccc(CNC(=O)c2cc(-c3cnsc3)cc3ccc(Cc4ccc5ccccc5c4)n23)cc1 ; C[C@H](NC(=O)c1cc(-c2ccc(F)cc2)cc2ccc(Cc3ccc4ccccc4c3F)n12)c1ccc(C(=O)O)cc1]

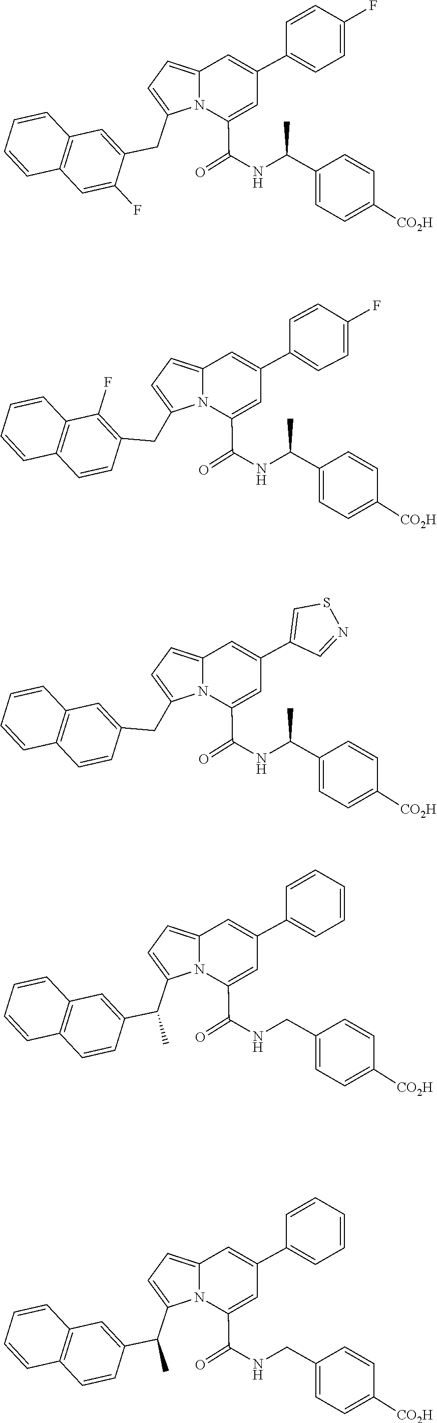 [SMILES: C[C@H](NC(=O)c1cc(-c2ccc(F)cc2)cc2ccc(Cc3cc4ccccc4cc3F)n12)c1ccc(C(=O)O)cc1 ; C[C@H](NC(=O)c1cc(-c2ccc(F)cc2)cc2ccc(Cc3ccc4ccccc4c3F)n12)c1ccc(C(=O)O)cc1 ; C[C@H](NC(=O)c1cc(-c2cnsc2)cc2ccc(Cc3ccc4ccccc4c3)n12)c1ccc(C(=O)O)cc1 ; C[C@H](c1ccc2ccccc2c1)c1ccc2cc(-c3ccccc3)cc(C(=O)NCc3ccc(C(=O)O)cc3)n12 ; C[C@@H](c1ccc2ccccc2c1)c1ccc2cc(-c3ccccc3)cc(C(=O)NCc3ccc(C(=O)O)cc3)n12]

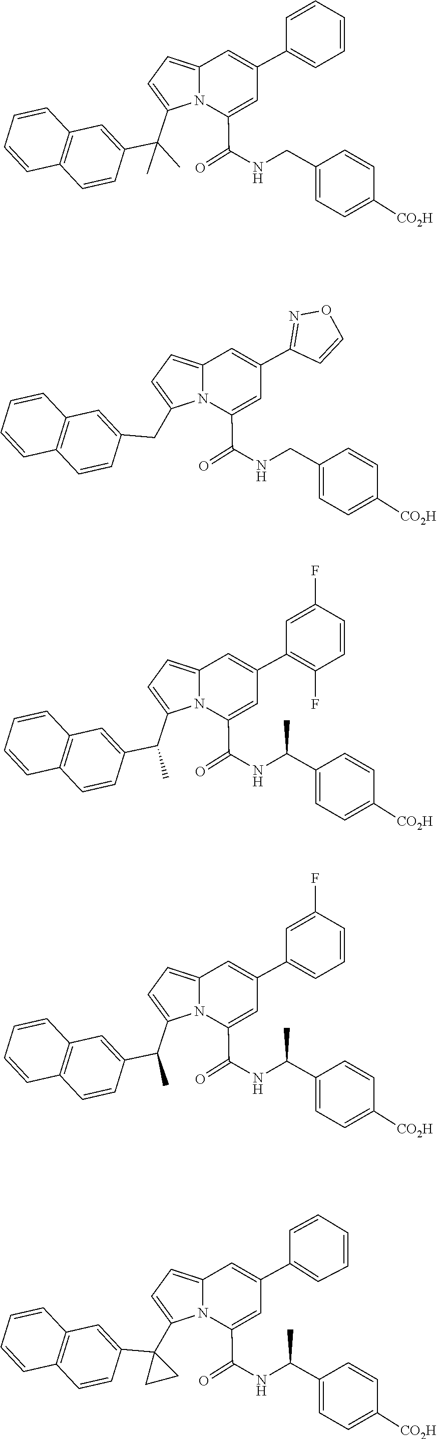 [SMILES: CC(C)(c1ccc2ccccc2c1)c1ccc2cc(-c3ccccc3)cc(C(=O)NCc3ccc(C(=O)O)cc3)n12 ; O=C(O)c1ccc(CNC(=O)c2cc(-c3ccon3)cc3ccc(Cc4ccc5ccccc5c4)n23)cc1 ; C[C@H](c1ccc2ccccc2c1)c1ccc2cc(-c3cc(F)ccc3F)cc(C(=O)N[C@@H](C)c3ccc(C(=O)O)cc3)n12 ; C[C@@H](c1ccc2ccccc2c1)c1ccc2cc(-c3cccc(F)c3)cc(C(=O)N[C@@H](C)c3ccc(C(=O)O)cc3)n12 ; C[C@H](NC(=O)c1cc(-c2ccccc2)cc2ccc(C3(c4ccc5ccccc5c4)CC3)n12)c1ccc(C(=O)O)cc1]

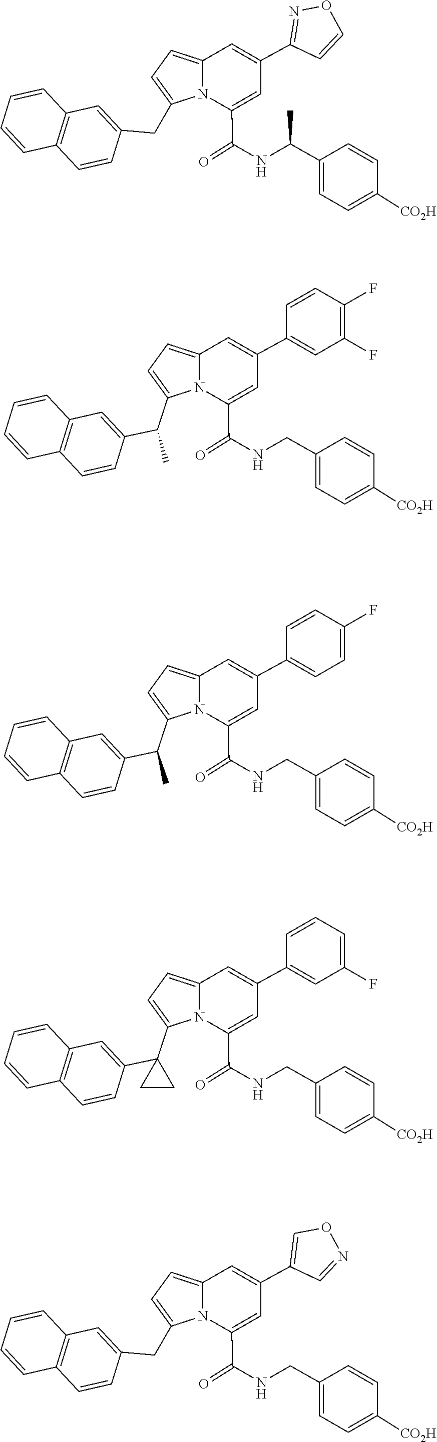 [SMILES: C[C@H](NC(=O)c1cc(-c2ccon2)cc2ccc(Cc3ccc4ccccc4c3)n12)c1ccc(C(=O)O)cc1 ; C[C@H](c1ccc2ccccc2c1)c1ccc2cc(-c3ccc(F)c(F)c3)cc(C(=O)NCc3ccc(C(=O)O)cc3)n12 ; C[C@@H](c1ccc2ccccc2c1)c1ccc2cc(-c3ccc(F)cc3)cc(C(=O)NCc3ccc(C(=O)O)cc3)n12 ; O=C(O)c1ccc(CNC(=O)c2cc(-c3cccc(F)c3)cc3ccc(C4(c5ccc6ccccc6c5)CC4)n23)cc1 ; O=C(O)c1ccc(CNC(=O)c2cc(-c3cnoc3)cc3ccc(Cc4ccc5ccccc5c4)n23)cc1]

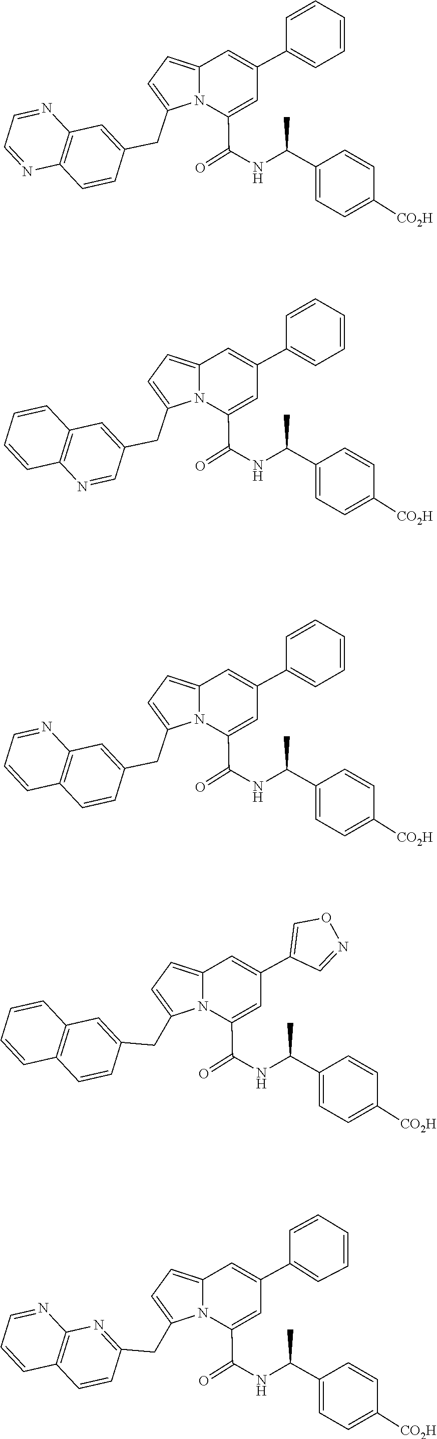 [SMILES: C[C@H](NC(=O)c1cc(-c2ccccc2)cc2ccc(Cc3ccc4nccnc4c3)n12)c1ccc(C(=O)O)cc1 ; C[C@H](NC(=O)c1cc(-c2ccccc2)cc2ccc(Cc3cnc4ccccc4c3)n12)c1ccc(C(=O)O)cc1 ; C[C@H](NC(=O)c1cc(-c2ccccc2)cc2ccc(Cc3ccc4cccnc4c3)n12)c1ccc(C(=O)O)cc1 ; C[C@H](NC(=O)c1cc(-c2cnoc2)cc2ccc(Cc3ccc4ccccc4c3)n12)c1ccc(C(=O)O)cc1 ; C[C@H](NC(=O)c1cc(-c2ccccc2)cc2ccc(Cc3ccc4cccnc4n3)n12)c1ccc(C(=O)O)cc1]

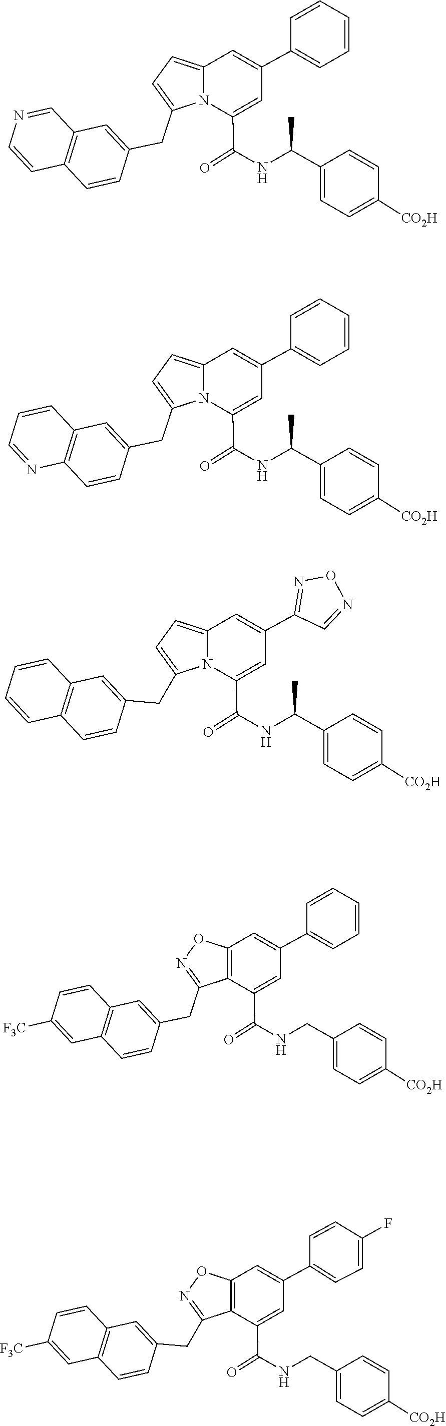 [SMILES: C[C@H](NC(=O)c1cc(-c2ccccc2)cc2ccc(Cc3ccc4ccncc4c3)n12)c1ccc(C(=O)O)cc1 ; C[C@H](NC(=O)c1cc(-c2ccccc2)cc2ccc(Cc3ccc4ncccc4c3)n12)c1ccc(C(=O)O)cc1 ; C[C@H](NC(=O)c1cc(-c2cnon2)cc2ccc(Cc3ccc4ccccc4c3)n12)c1ccc(C(=O)O)cc1 ; O=C(O)c1ccc(CNC(=O)c2cc(-c3ccccc3)cc3onc(Cc4ccc5cc(C(F)(F)F)ccc5c4)c23)cc1 ; O=C(O)c1ccc(CNC(=O)c2cc(-c3ccc(F)cc3)cc3onc(Cc4ccc5cc(C(F)(F)F)ccc5c4)c23)cc1]

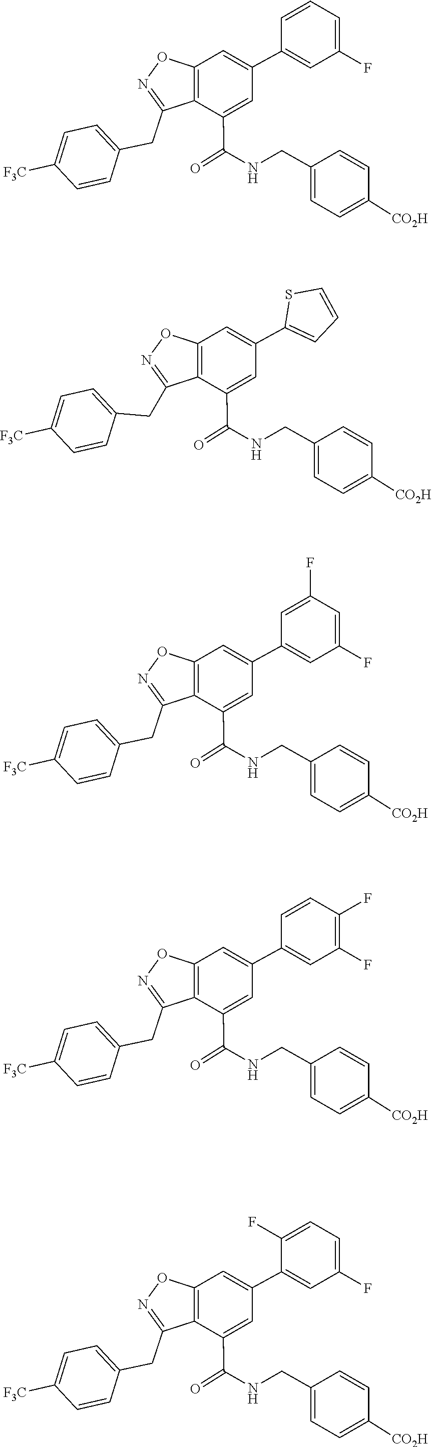 [SMILES: O=C(O)c1ccc(CNC(=O)c2cc(-c3cccc(F)c3)cc3onc(Cc4ccc(C(F)(F)F)cc4)c23)cc1 ; O=C(O)c1ccc(CNC(=O)c2cc(-c3cccs3)cc3onc(Cc4ccc(C(F)(F)F)cc4)c23)cc1 ; O=C(O)c1ccc(CNC(=O)c2cc(-c3cc(F)cc(F)c3)cc3onc(Cc4ccc(C(F)(F)F)cc4)c23)cc1 ; O=C(O)c1ccc(CNC(=O)c2cc(-c3ccc(F)c(F)c3)cc3onc(Cc4ccc(C(F)(F)F)cc4)c23)cc1 ; O=C(O)c1ccc(CNC(=O)c2cc(-c3cc(F)ccc3F)cc3onc(Cc4ccc(C(F)(F)F)cc4)c23)cc1]

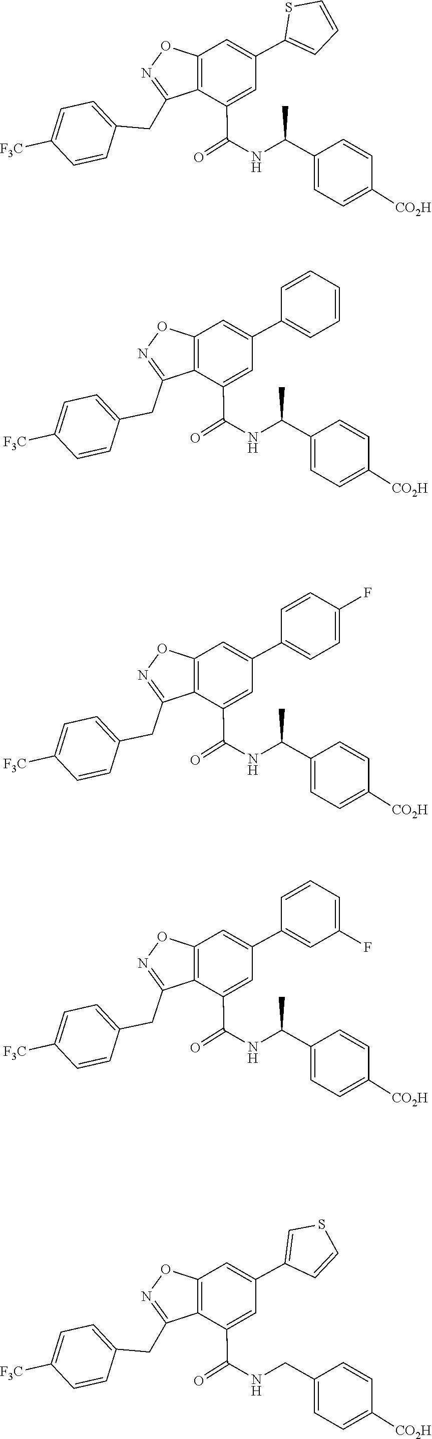 [SMILES: C[C@H](NC(=O)c1cc(-c2cccs2)cc2onc(Cc3ccc(C(F)(F)F)cc3)c12)c1ccc(C(=O)O)cc1 ; C[C@H](NC(=O)c1cc(-c2ccccc2)cc2onc(Cc3ccc(C(F)(F)F)cc3)c12)c1ccc(C(=O)O)cc1 ; C[C@H](NC(=O)c1cc(-c2ccc(F)cc2)cc2onc(Cc3ccc(C(F)(F)F)cc3)c12)c1ccc(C(=O)O)cc1 ; C[C@H](NC(=O)c1cc(-c2cccc(F)c2)cc2onc(Cc3ccc(C(F)(F)F)cc3)c12)c1ccc(C(=O)O)cc1 ; O=C(O)c1ccc(CNC(=O)c2cc(-c3ccsc3)cc3onc(Cc4ccc(C(F)(F)F)cc4)c23)cc1]

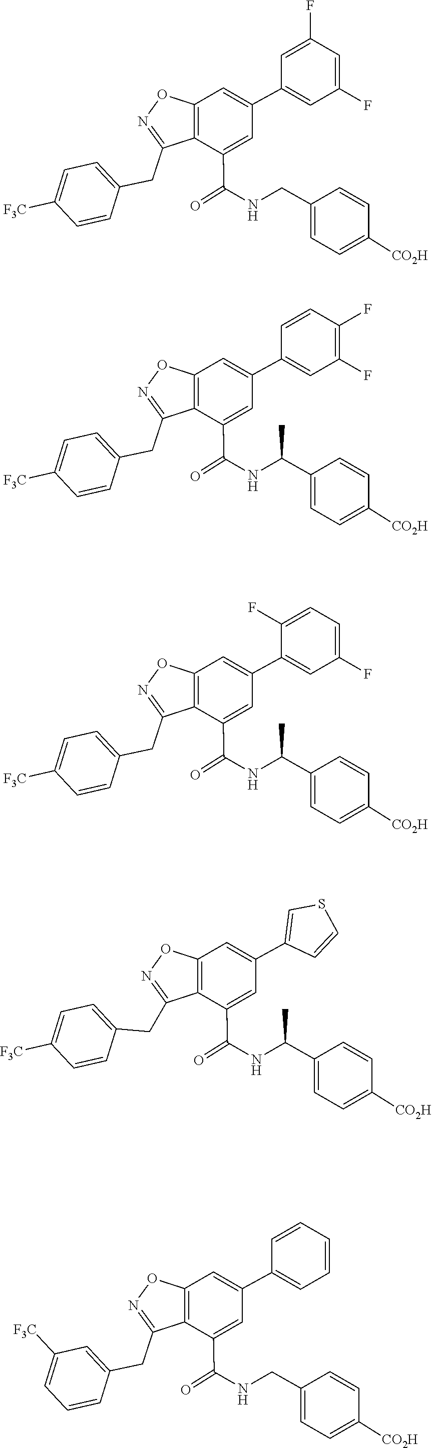 [SMILES: O=C(O)c1ccc(CNC(=O)c2cc(-c3cc(F)cc(F)c3)cc3onc(Cc4ccc(C(F)(F)F)cc4)c23)cc1 ; C[C@H](NC(=O)c1cc(-c2ccc(F)c(F)c2)cc2onc(Cc3ccc(C(F)(F)F)cc3)c12)c1ccc(C(=O)O)cc1 ; C[C@H](NC(=O)c1cc(-c2cc(F)ccc2F)cc2onc(Cc3ccc(C(F)(F)F)cc3)c12)c1ccc(C(=O)O)cc1 ; C[C@H](NC(=O)c1cc(-c2ccsc2)cc2onc(Cc3ccc(C(F)(F)F)cc3)c12)c1ccc(C(=O)O)cc1 ; O=C(O)c1ccc(CNC(=O)c2cc(-c3ccccc3)cc3onc(Cc4cccc(C(F)(F)F)c4)c23)cc1]

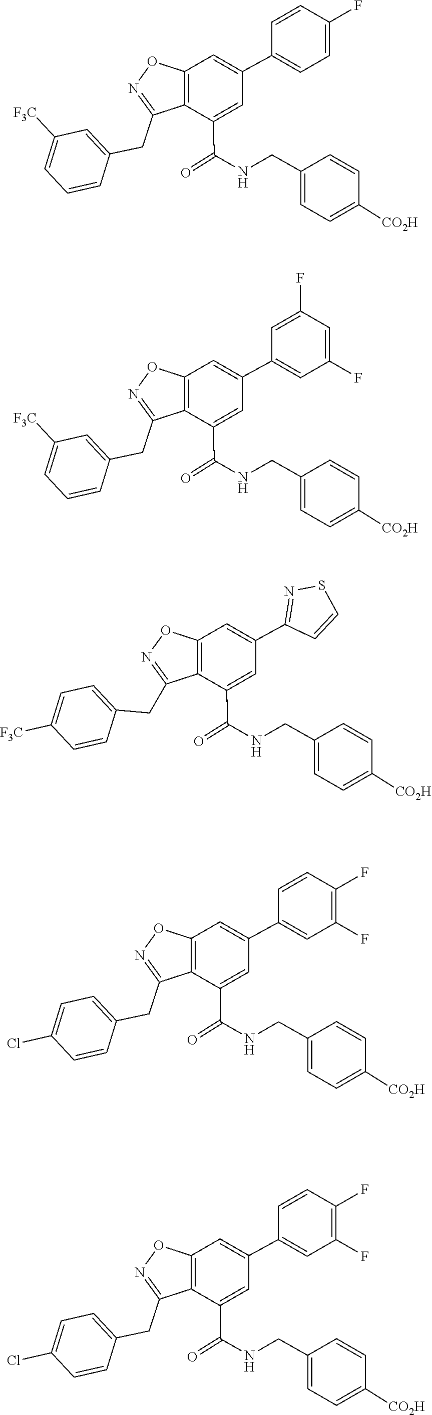 [SMILES: O=C(O)c1ccc(CNC(=O)c2cc(-c3ccc(F)cc3)cc3onc(Cc4cccc(C(F)(F)F)c4)c23)cc1 ; O=C(O)c1ccc(CNC(=O)c2cc(-c3cc(F)cc(F)c3)cc3onc(Cc4cccc(C(F)(F)F)c4)c23)cc1 ; O=C(O)c1ccc(CNC(=O)c2cc(-c3ccsn3)cc3onc(Cc4ccc(C(F)(F)F)cc4)c23)cc1 ; O=C(O)c1ccc(CNC(=O)c2cc(-c3ccc(F)c(F)c3)cc3onc(Cc4ccc(Cl)cc4)c23)cc1 ; O=C(O)c1ccc(CNC(=O)c2cc(-c3ccc(F)c(F)c3)cc3onc(Cc4ccc(Cl)cc4)c23)cc1]

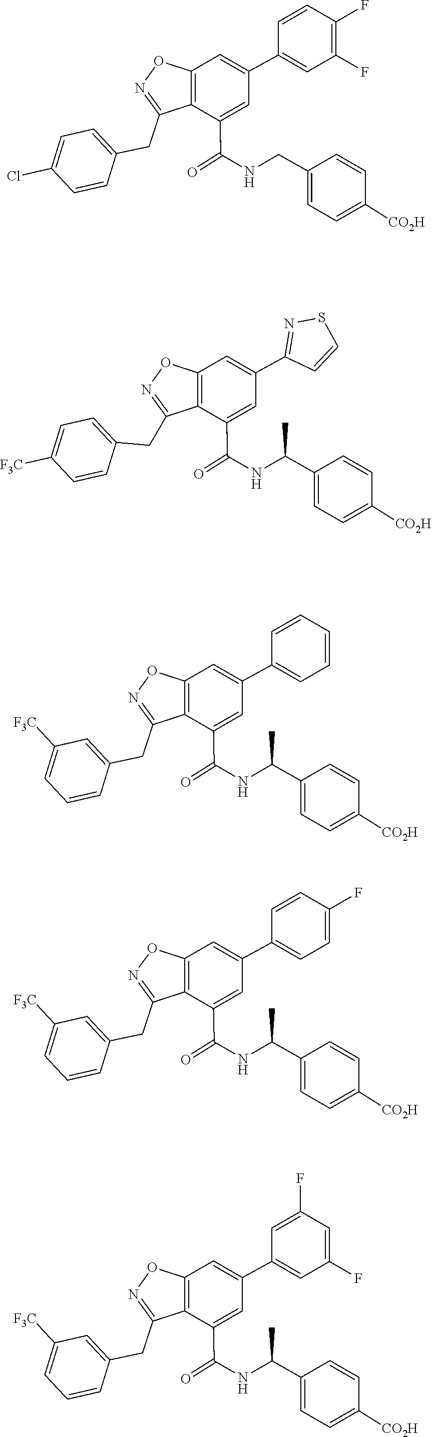 [SMILES: O=C(O)c1ccc(CNC(=O)c2cc(-c3ccc(F)c(F)c3)cc3onc(Cc4ccc(Cl)cc4)c23)cc1 ; C[C@H](NC(=O)c1cc(-c2ccsn2)cc2onc(Cc3ccc(C(F)(F)F)cc3)c12)c1ccc(C(=O)O)cc1 ; C[C@H](NC(=O)c1cc(-c2ccccc2)cc2onc(Cc3cccc(C(F)(F)F)c3)c12)c1ccc(C(=O)O)cc1 ; C[C@H](NC(=O)c1cc(-c2ccc(F)cc2)cc2onc(Cc3cccc(C(F)(F)F)c3)c12)c1ccc(C(=O)O)cc1 ; C[C@H](NC(=O)c1cc(-c2cc(F)cc(F)c2)cc2onc(Cc3cccc(C(F)(F)F)c3)c12)c1ccc(C(=O)O)cc1]

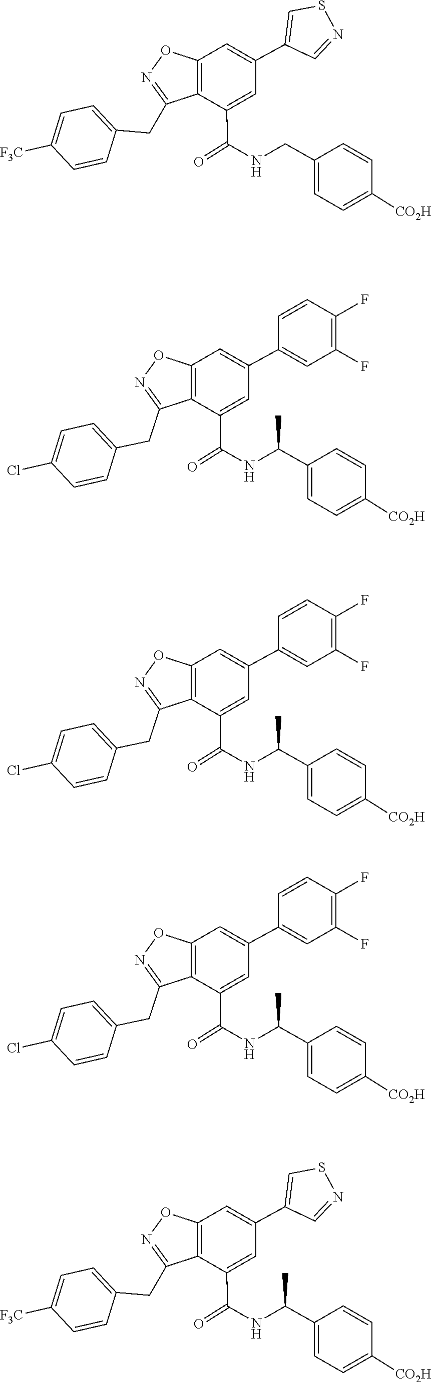 [SMILES: O=C(O)c1ccc(CNC(=O)c2cc(-c3cnsc3)cc3onc(Cc4ccc(C(F)(F)F)cc4)c23)cc1 ; C[C@H](NC(=O)c1cc(-c2ccc(F)c(F)c2)cc2onc(Cc3ccc(Cl)cc3)c12)c1ccc(C(=O)O)cc1 ; C[C@H](NC(=O)c1cc(-c2ccc(F)c(F)c2)cc2onc(Cc3ccc(Cl)cc3)c12)c1ccc(C(=O)O)cc1 ; C[C@H](NC(=O)c1cc(-c2ccc(F)c(F)c2)cc2onc(Cc3ccc(Cl)cc3)c12)c1ccc(C(=O)O)cc1 ; C[C@H](NC(=O)c1cc(-c2cnsc2)cc2onc(Cc3ccc(C(F)(F)F)cc3)c12)c1ccc(C(=O)O)cc1]

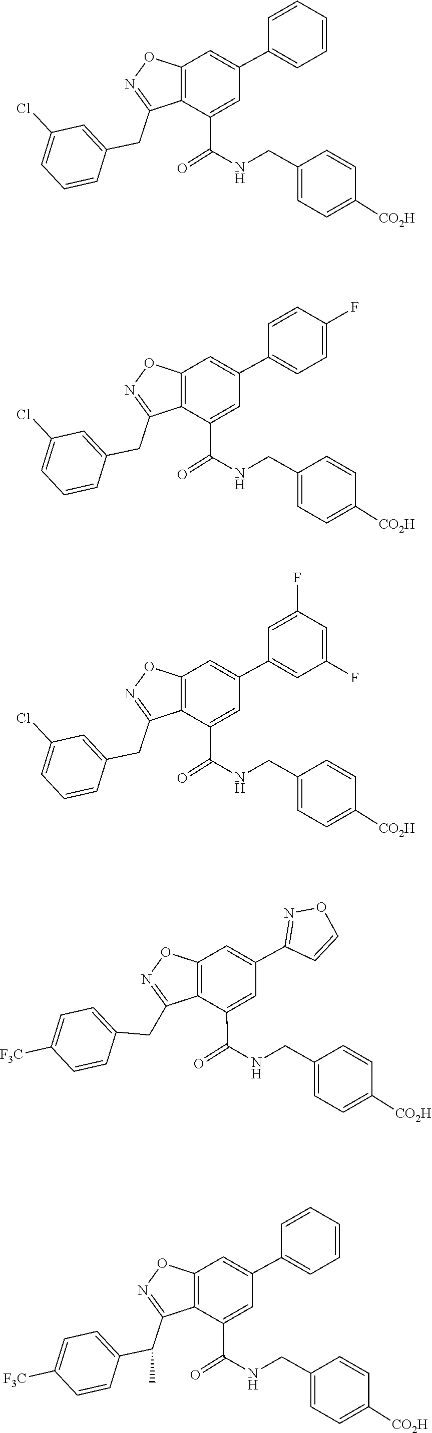 [SMILES: O=C(O)c1ccc(CNC(=O)c2cc(-c3ccccc3)cc3onc(Cc4cccc(Cl)c4)c23)cc1 ; O=C(O)c1ccc(CNC(=O)c2cc(-c3ccc(F)cc3)cc3onc(Cc4cccc(Cl)c4)c23)cc1 ; O=C(O)c1ccc(CNC(=O)c2cc(-c3cc(F)cc(F)c3)cc3onc(Cc4cccc(Cl)c4)c23)cc1 ; O=C(O)c1ccc(CNC(=O)c2cc(-c3ccon3)cc3onc(Cc4ccc(C(F)(F)F)cc4)c23)cc1 ; C[C@H](c1ccc(C(F)(F)F)cc1)c1noc2cc(-c3ccccc3)cc(C(=O)NCc3ccc(C(=O)O)cc3)c12]

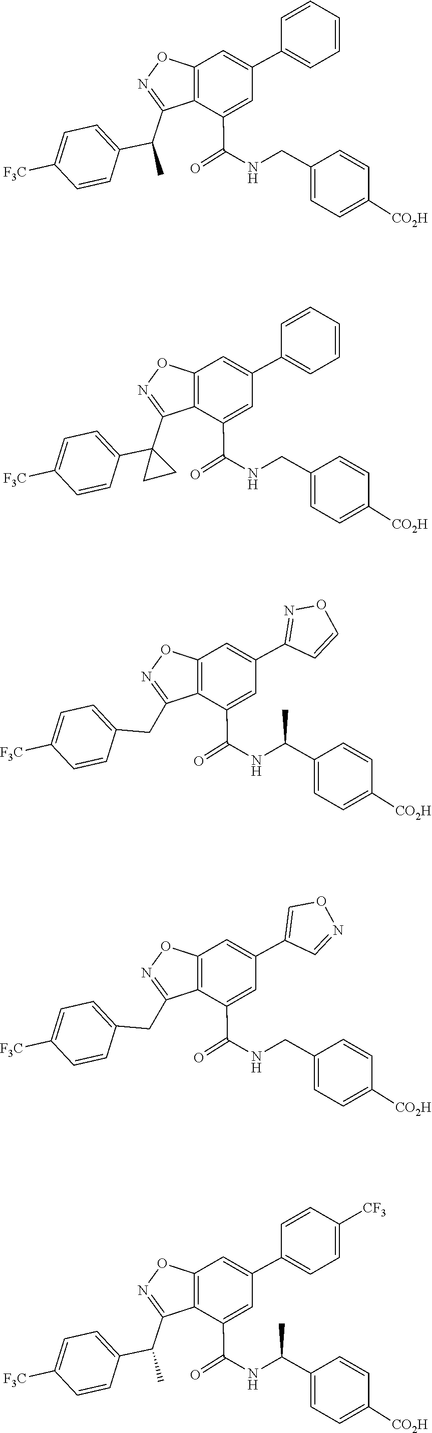 [SMILES: C[C@@H](c1ccc(C(F)(F)F)cc1)c1noc2cc(-c3ccccc3)cc(C(=O)NCc3ccc(C(=O)O)cc3)c12 ; O=C(O)c1ccc(CNC(=O)c2cc(-c3ccccc3)cc3onc(C4(c5ccc(C(F)(F)F)cc5)CC4)c23)cc1 ; C[C@H](NC(=O)c1cc(-c2ccon2)cc2onc(Cc3ccc(C(F)(F)F)cc3)c12)c1ccc(C(=O)O)cc1 ; O=C(O)c1ccc(CNC(=O)c2cc(-c3cnoc3)cc3onc(Cc4ccc(C(F)(F)F)cc4)c23)cc1 ; C[C@H](c1ccc(C(F)(F)F)cc1)c1noc2cc(-c3ccc(C(F)(F)F)cc3)cc(C(=O)N[C@@H](C)c3ccc(C(=O)O)cc3)c12]

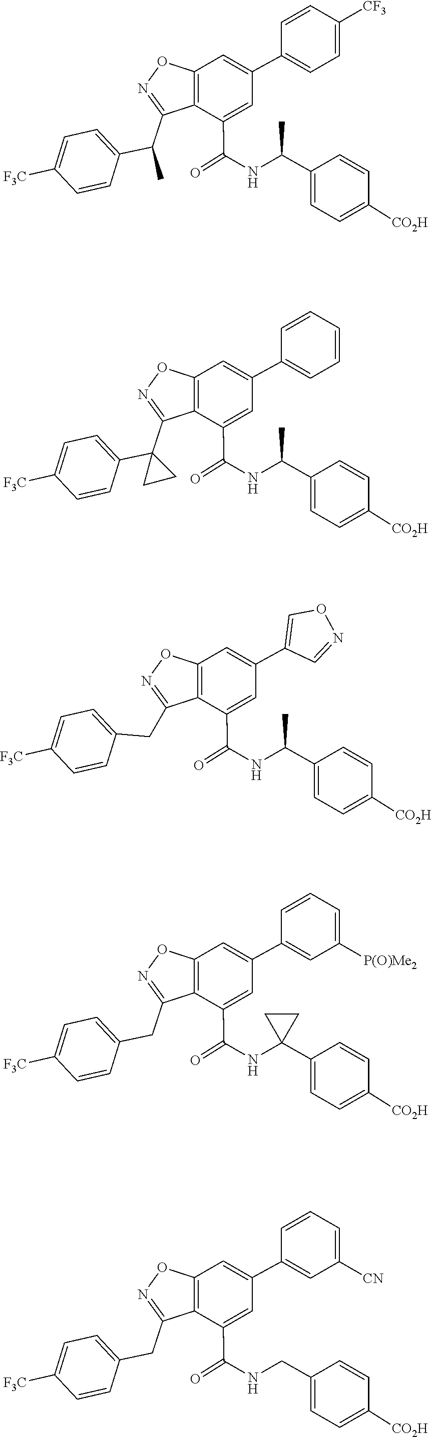

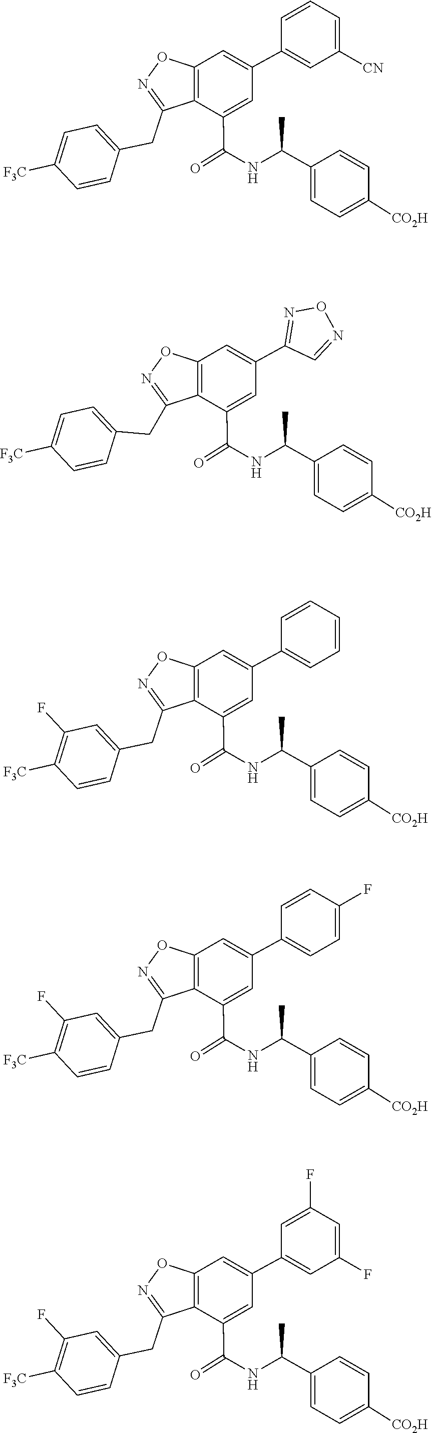 [SMILES: C[C@H](NC(=O)c1cc(-c2cccc(C#N)c2)cc2onc(Cc3ccc(C(F)(F)F)cc3)c12)c1ccc(C(=O)O)cc1 ; C[C@H](NC(=O)c1cc(-c2cnon2)cc2onc(Cc3ccc(C(F)(F)F)cc3)c12)c1ccc(C(=O)O)cc1 ; C[C@H](NC(=O)c1cc(-c2ccccc2)cc2onc(Cc3ccc(C(F)(F)F)c(F)c3)c12)c1ccc(C(=O)O)cc1 ; C[C@H](NC(=O)c1cc(-c2ccc(F)cc2)cc2onc(Cc3ccc(C(F)(F)F)c(F)c3)c12)c1ccc(C(=O)O)cc1 ; C[C@H](NC(=O)c1cc(-c2cc(F)cc(F)c2)cc2onc(Cc3ccc(C(F)(F)F)c(F)c3)c12)c1ccc(C(=O)O)cc1]

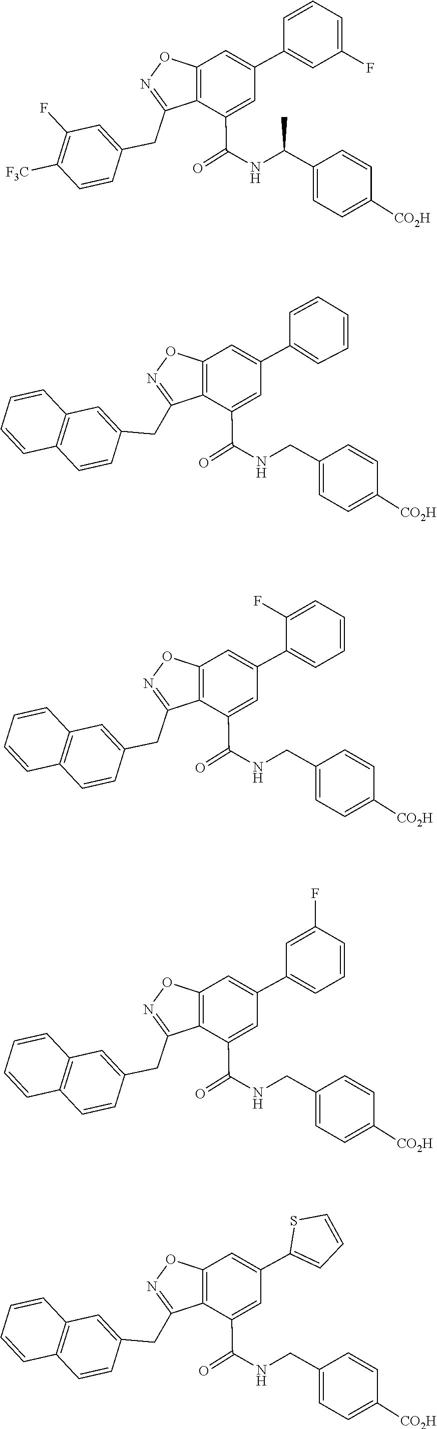 [SMILES: C[C@H](NC(=O)c1cc(-c2cccc(F)c2)cc2onc(Cc3ccc(C(F)(F)F)c(F)c3)c12)c1ccc(C(=O)O)cc1 ; O=C(O)c1ccc(CNC(=O)c2cc(-c3ccccc3)cc3onc(Cc4ccc5ccccc5c4)c23)cc1 ; O=C(O)c1ccc(CNC(=O)c2cc(-c3ccccc3F)cc3onc(Cc4ccc5ccccc5c4)c23)cc1 ; O=C(O)c1ccc(CNC(=O)c2cc(-c3cccc(F)c3)cc3onc(Cc4ccc5ccccc5c4)c23)cc1 ; O=C(O)c1ccc(CNC(=O)c2cc(-c3cccs3)cc3onc(Cc4ccc5ccccc5c4)c23)cc1]

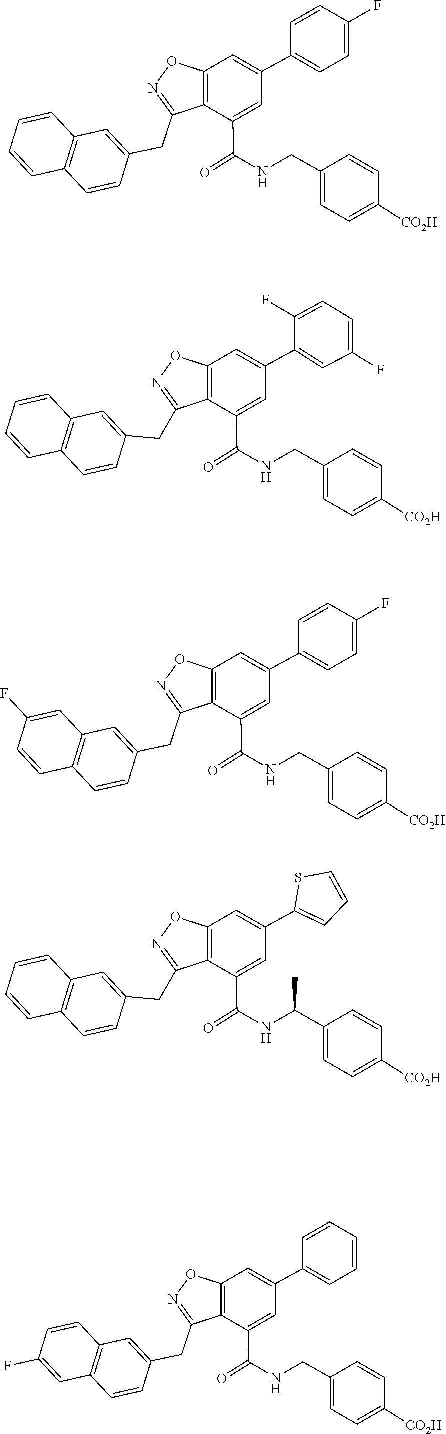 [SMILES: O=C(O)c1ccc(CNC(=O)c2cc(-c3ccc(F)cc3)cc3onc(Cc4ccc5ccccc5c4)c23)cc1 ; O=C(O)c1ccc(CNC(=O)c2cc(-c3cc(F)ccc3F)cc3onc(Cc4ccc5ccccc5c4)c23)cc1 ; O=C(O)c1ccc(CNC(=O)c2cc(-c3ccc(F)cc3)cc3onc(Cc4ccc5ccc(F)cc5c4)c23)cc1 ; C[C@H](NC(=O)c1cc(-c2cccs2)cc2onc(Cc3ccc4ccccc4c3)c12)c1ccc(C(=O)O)cc1 ; O=C(O)c1ccc(CNC(=O)c2cc(-c3ccccc3)cc3onc(Cc4ccc5cc(F)ccc5c4)c23)cc1]

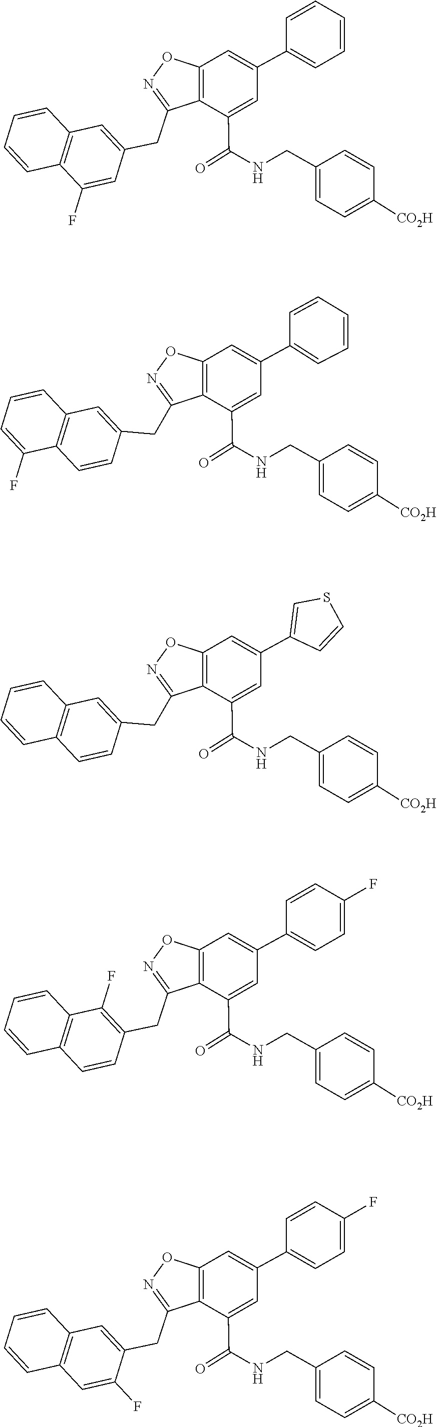 [SMILES: O=C(O)c1ccc(CNC(=O)c2cc(-c3ccccc3)cc3onc(Cc4cc(F)c5ccccc5c4)c23)cc1 ; O=C(O)c1ccc(CNC(=O)c2cc(-c3ccccc3)cc3onc(Cc4ccc5c(F)cccc5c4)c23)cc1 ; O=C(O)c1ccc(CNC(=O)c2cc(-c3ccsc3)cc3onc(Cc4ccc5ccccc5c4)c23)cc1 ; O=C(O)c1ccc(CNC(=O)c2cc(-c3ccc(F)cc3)cc3onc(Cc4ccc5ccccc5c4F)c23)cc1 ; O=C(O)c1ccc(CNC(=O)c2cc(-c3ccc(F)cc3)cc3onc(Cc4cc5ccccc5cc4F)c23)cc1]

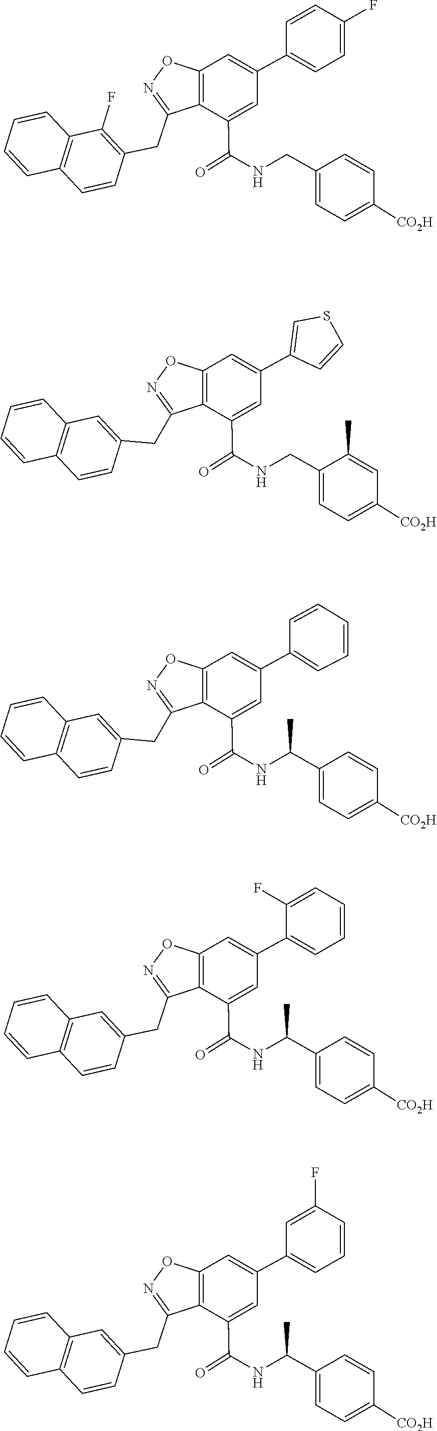

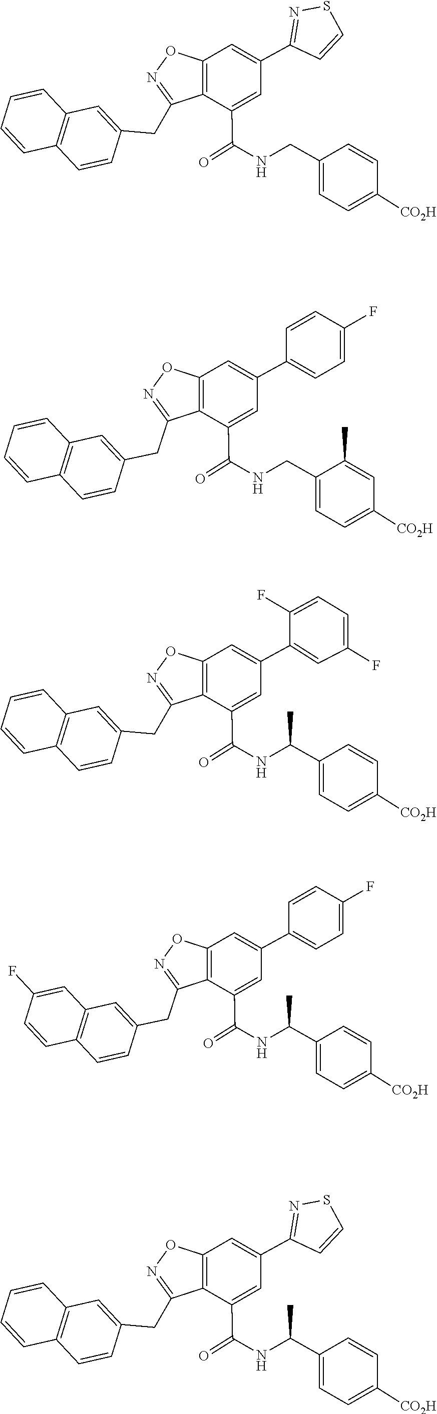

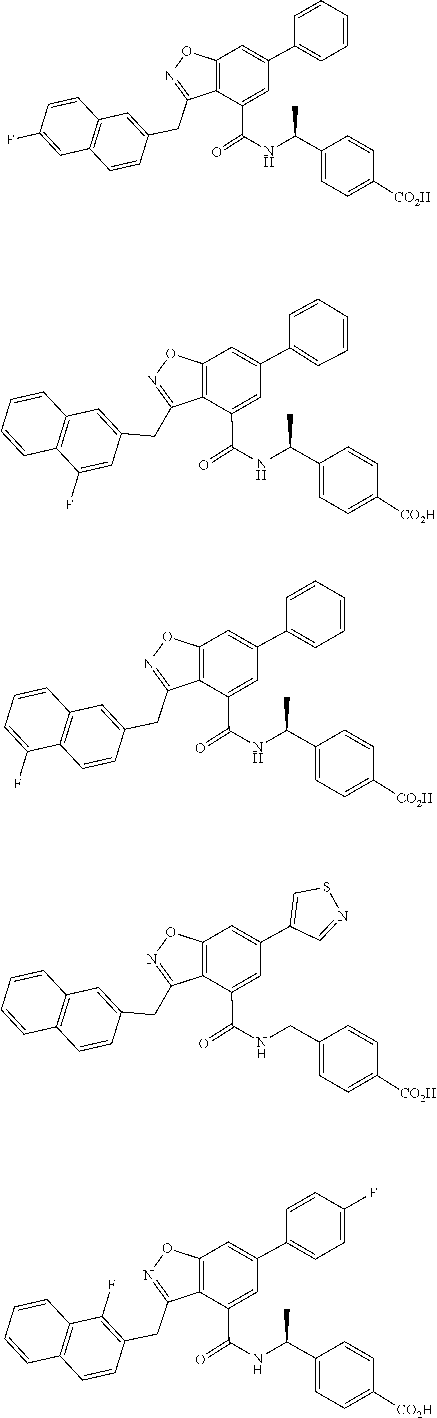 [SMILES: C[C@H](NC(=O)c1cc(-c2ccccc2)cc2onc(Cc3ccc4cc(F)ccc4c3)c12)c1ccc(C(=O)O)cc1 ; C[C@H](NC(=O)c1cc(-c2ccccc2)cc2onc(Cc3cc(F)c4ccccc4c3)c12)c1ccc(C(=O)O)cc1 ; C[C@H](NC(=O)c1cc(-c2ccccc2)cc2onc(Cc3ccc4c(F)cccc4c3)c12)c1ccc(C(=O)O)cc1 ; O=C(O)c1ccc(CNC(=O)c2cc(-c3cnsc3)cc3onc(Cc4ccc5ccccc5c4)c23)cc1 ; C[C@H](NC(=O)c1cc(-c2ccc(F)cc2)cc2onc(Cc3ccc4ccccc4c3F)c12)c1ccc(C(=O)O)cc1]

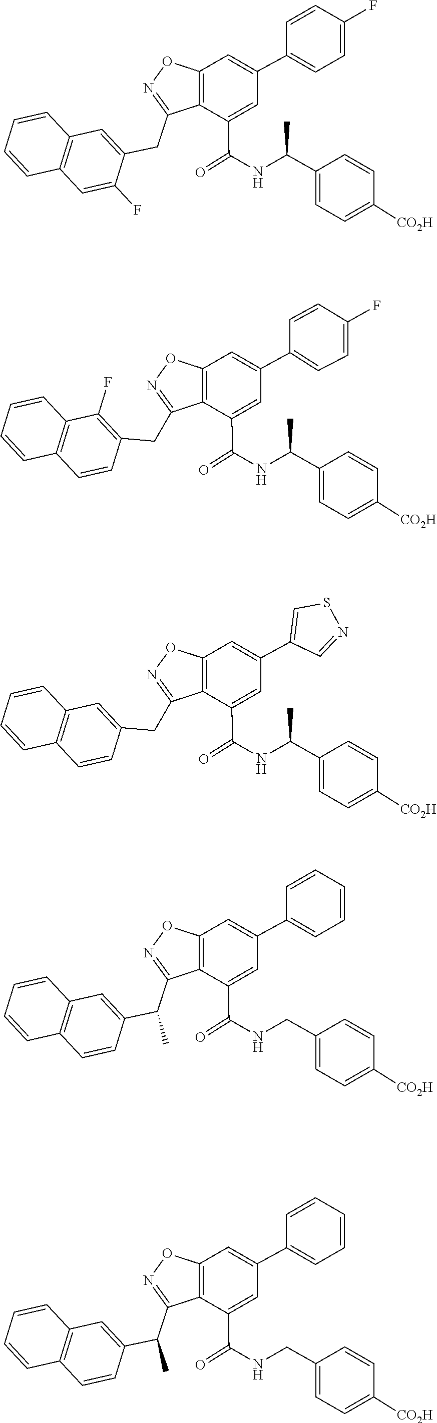 [SMILES: C[C@H](NC(=O)c1cc(-c2ccc(F)cc2)cc2onc(Cc3cc4ccccc4cc3F)c12)c1ccc(C(=O)O)cc1 ; C[C@H](NC(=O)c1cc(-c2ccc(F)cc2)cc2onc(Cc3ccc4ccccc4c3F)c12)c1ccc(C(=O)O)cc1 ; C[C@H](NC(=O)c1cc(-c2cnsc2)cc2onc(Cc3ccc4ccccc4c3)c12)c1ccc(C(=O)O)cc1 ; C[C@H](c1ccc2ccccc2c1)c1noc2cc(-c3ccccc3)cc(C(=O)NCc3ccc(C(=O)O)cc3)c12 ; C[C@@H](c1ccc2ccccc2c1)c1noc2cc(-c3ccccc3)cc(C(=O)NCc3ccc(C(=O)O)cc3)c12]

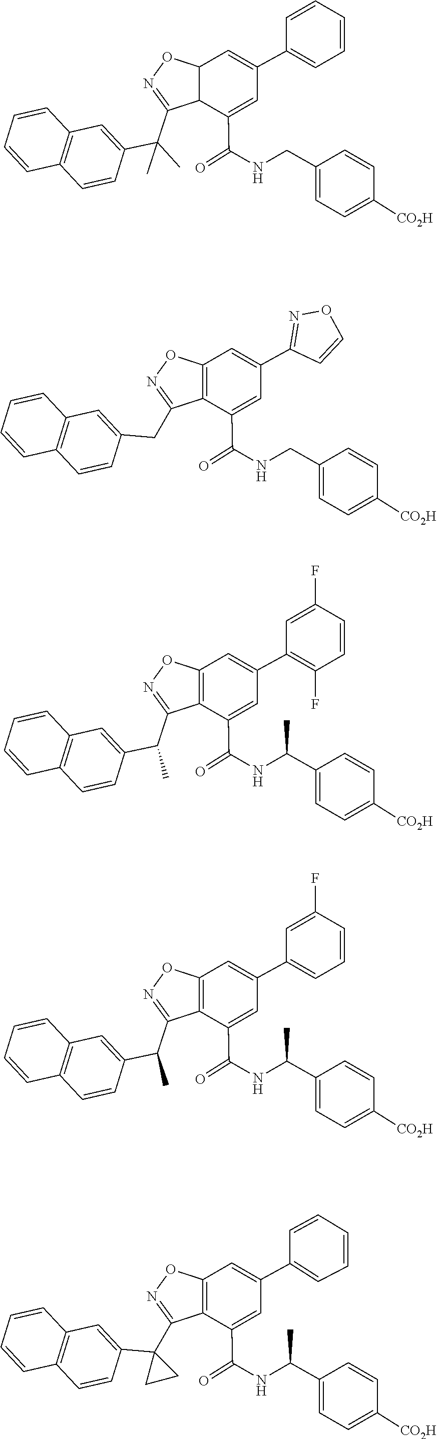 [SMILES: CC(C)(C1=NOC2C=C(c3ccccc3)C=C(C(=O)NCc3ccc(C(=O)O)cc3)C12)c1ccc2ccccc2c1 ; O=C(O)c1ccc(CNC(=O)c2cc(-c3ccon3)cc3onc(Cc4ccc5ccccc5c4)c23)cc1 ; C[C@H](c1ccc2ccccc2c1)c1noc2cc(-c3cc(F)ccc3F)cc(C(=O)N[C@@H](C)c3ccc(C(=O)O)cc3)c12 ; C[C@@H](c1ccc2ccccc2c1)c1noc2cc(-c3cccc(F)c3)cc(C(=O)N[C@@H](C)c3ccc(C(=O)O)cc3)c12 ; C[C@H](NC(=O)c1cc(-c2ccccc2)cc2onc(C3(c4ccc5ccccc5c4)CC3)c12)c1ccc(C(=O)O)cc1]

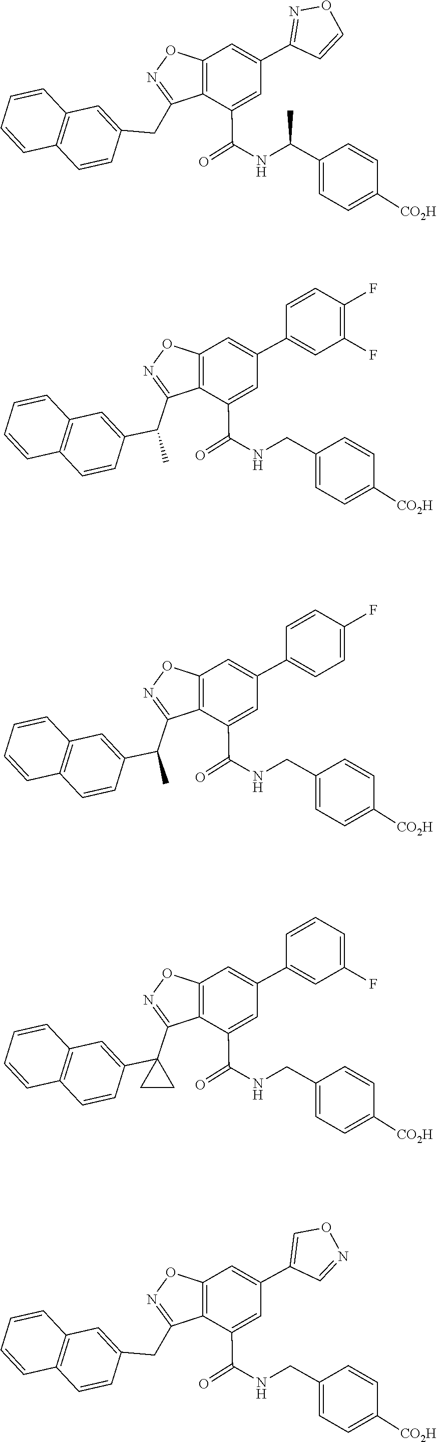 [SMILES: C[C@H](NC(=O)c1cc(-c2ccon2)cc2onc(Cc3ccc4ccccc4c3)c12)c1ccc(C(=O)O)cc1 ; C[C@H](c1ccc2ccccc2c1)c1noc2cc(-c3ccc(F)c(F)c3)cc(C(=O)NCc3ccc(C(=O)O)cc3)c12 ; C[C@@H](c1ccc2ccccc2c1)c1noc2cc(-c3ccc(F)cc3)cc(C(=O)NCc3ccc(C(=O)O)cc3)c12 ; O=C(O)c1ccc(CNC(=O)c2cc(-c3cccc(F)c3)cc3onc(C4(c5ccc6ccccc6c5)CC4)c23)cc1 ; O=C(O)c1ccc(CNC(=O)c2cc(-c3cnoc3)cc3onc(Cc4ccc5ccccc5c4)c23)cc1]

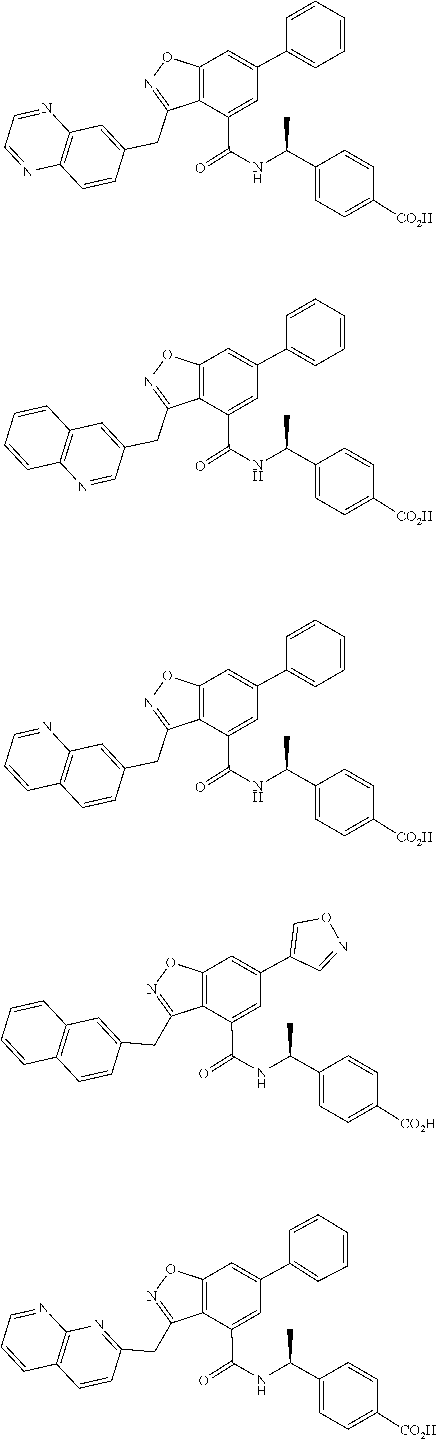 [SMILES: C[C@H](NC(=O)c1cc(-c2ccccc2)cc2onc(Cc3ccc4nccnc4c3)c12)c1ccc(C(=O)O)cc1 ; C[C@H](NC(=O)c1cc(-c2ccccc2)cc2onc(Cc3cnc4ccccc4c3)c12)c1ccc(C(=O)O)cc1 ; C[C@H](NC(=O)c1cc(-c2ccccc2)cc2onc(Cc3ccc4cccnc4c3)c12)c1ccc(C(=O)O)cc1 ; C[C@H](NC(=O)c1cc(-c2cnoc2)cc2onc(Cc3ccc4ccccc4c3)c12)c1ccc(C(=O)O)cc1 ; C[C@H](NC(=O)c1cc(-c2ccccc2)cc2onc(Cc3ccc4cccnc4n3)c12)c1ccc(C(=O)O)cc1]

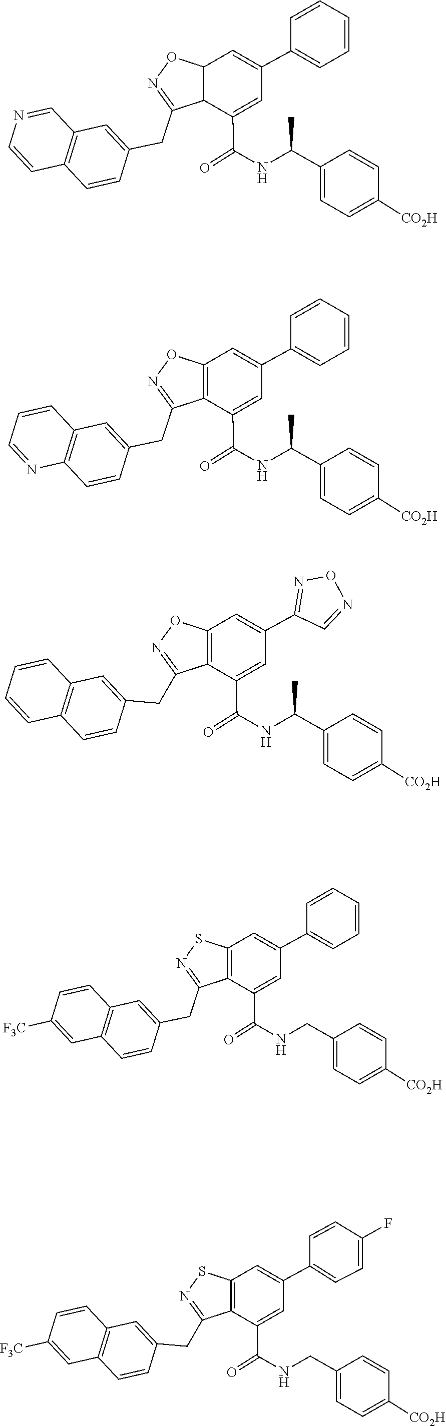 [SMILES: C[C@H](NC(=O)C1=CC(c2ccccc2)=CC2ON=C(Cc3ccc4ccncc4c3)C12)c1ccc(C(=O)O)cc1 ; C[C@H](NC(=O)c1cc(-c2ccccc2)cc2onc(Cc3ccc4ncccc4c3)c12)c1ccc(C(=O)O)cc1 ; C[C@H](NC(=O)c1cc(-c2cnon2)cc2onc(Cc3ccc4ccccc4c3)c12)c1ccc(C(=O)O)cc1 ; O=C(O)c1ccc(CNC(=O)c2cc(-c3ccccc3)cc3snc(Cc4ccc5cc(C(F)(F)F)ccc5c4)c23)cc1 ; O=C(O)c1ccc(CNC(=O)c2cc(-c3ccc(F)cc3)cc3snc(Cc4ccc5cc(C(F)(F)F)ccc5c4)c23)cc1]

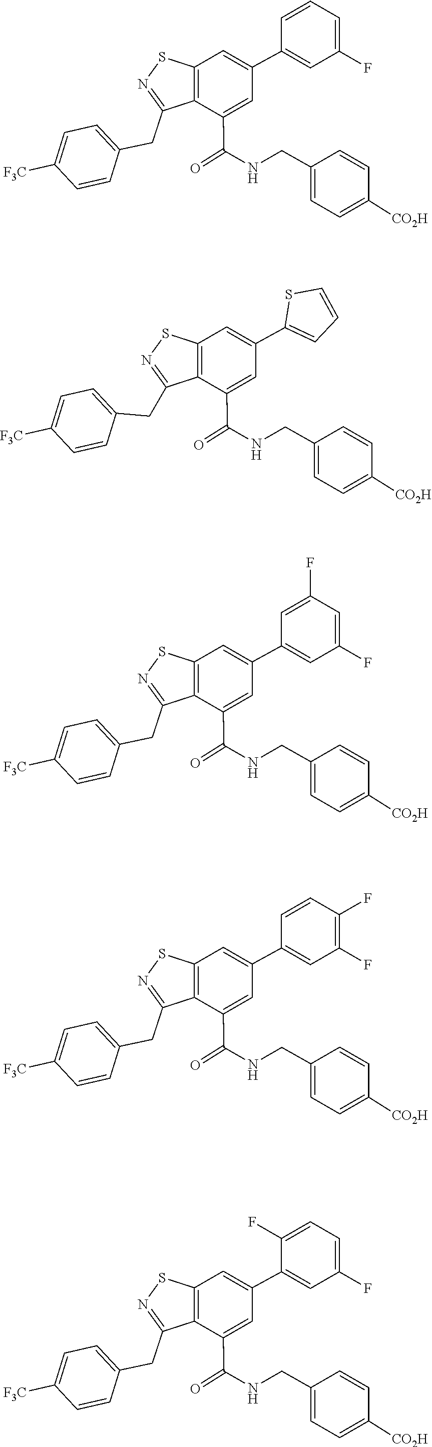 [SMILES: O=C(O)c1ccc(CNC(=O)c2cc(-c3cccc(F)c3)cc3snc(Cc4ccc(C(F)(F)F)cc4)c23)cc1 ; O=C(O)c1ccc(CNC(=O)c2cc(-c3cccs3)cc3snc(Cc4ccc(C(F)(F)F)cc4)c23)cc1 ; O=C(O)c1ccc(CNC(=O)c2cc(-c3cc(F)cc(F)c3)cc3snc(Cc4ccc(C(F)(F)F)cc4)c23)cc1 ; O=C(O)c1ccc(CNC(=O)c2cc(-c3ccc(F)c(F)c3)cc3snc(Cc4ccc(C(F)(F)F)cc4)c23)cc1 ; O=C(O)c1ccc(CNC(=O)c2cc(-c3cc(F)ccc3F)cc3snc(Cc4ccc(C(F)(F)F)cc4)c23)cc1]

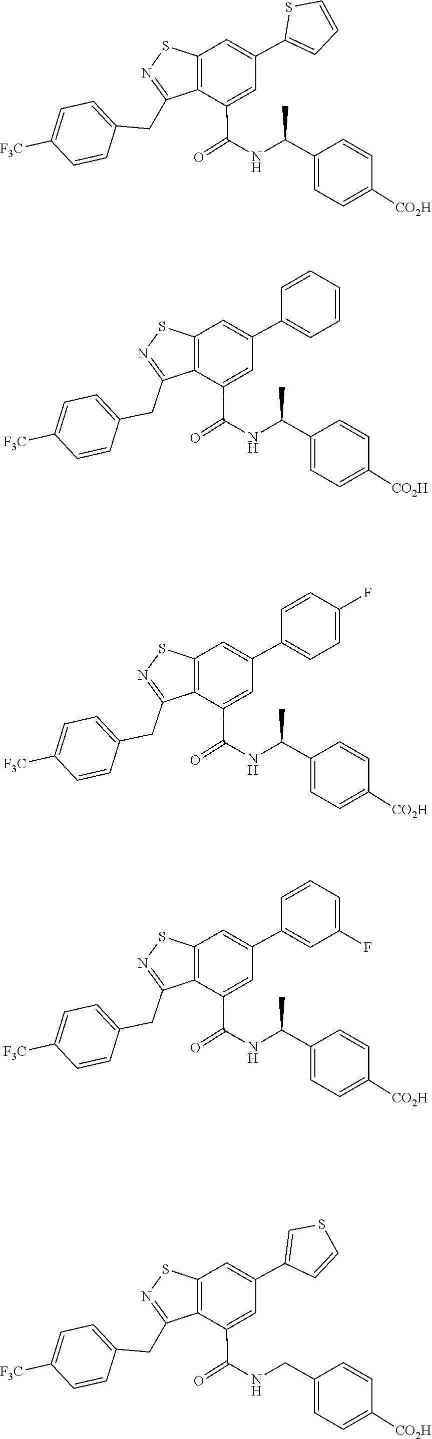 [SMILES: C[C@H](NC(=O)c1cc(-c2cccs2)cc2snc(Cc3ccc(C(F)(F)F)cc3)c12)c1ccc(C(=O)O)cc1 ; C[C@H](NC(=O)c1cc(-c2ccccc2)cc2snc(Cc3ccc(C(F)(F)F)cc3)c12)c1ccc(C(=O)O)cc1 ; C[C@H](NC(=O)c1cc(-c2ccc(F)cc2)cc2snc(Cc3ccc(C(F)(F)F)cc3)c12)c1ccc(C(=O)O)cc1 ; C[C@H](NC(=O)c1cc(-c2cccc(F)c2)cc2snc(Cc3ccc(C(F)(F)F)cc3)c12)c1ccc(C(=O)O)cc1 ; O=C(O)c1ccc(CNC(=O)c2cc(-c3ccsc3)cc3snc(Cc4ccc(C(F)(F)F)cc4)c23)cc1]

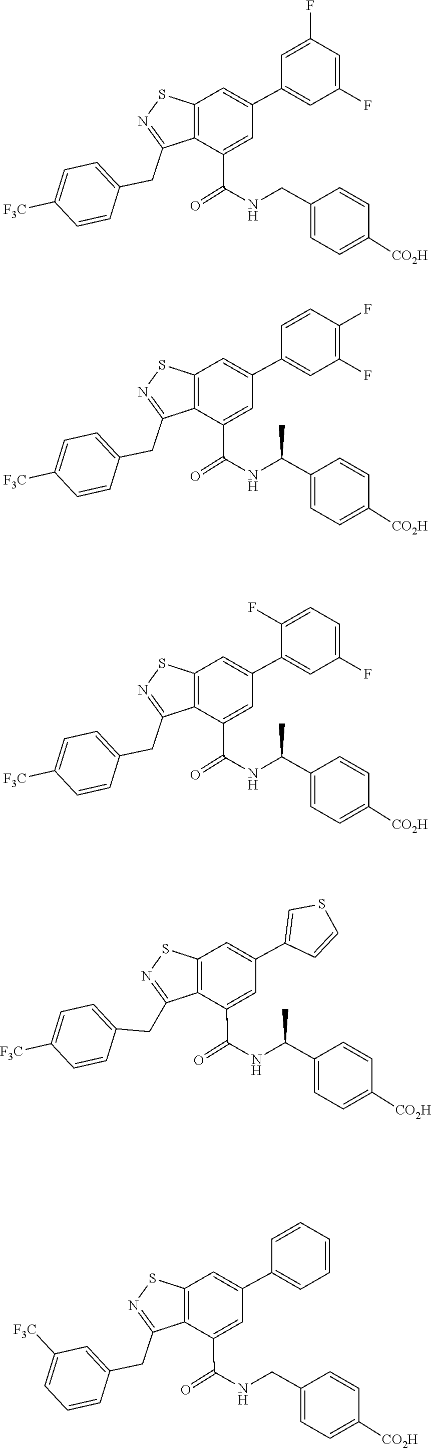 [SMILES: O=C(O)c1ccc(CNC(=O)c2cc(-c3cc(F)cc(F)c3)cc3snc(Cc4ccc(C(F)(F)F)cc4)c23)cc1 ; C[C@H](NC(=O)c1cc(-c2ccc(F)c(F)c2)cc2snc(Cc3ccc(C(F)(F)F)cc3)c12)c1ccc(C(=O)O)cc1 ; C[C@H](NC(=O)c1cc(-c2cc(F)ccc2F)cc2snc(Cc3ccc(C(F)(F)F)cc3)c12)c1ccc(C(=O)O)cc1 ; C[C@H](NC(=O)c1cc(-c2ccsc2)cc2snc(Cc3ccc(C(F)(F)F)cc3)c12)c1ccc(C(=O)O)cc1 ; O=C(O)c1ccc(CNC(=O)c2cc(-c3ccccc3)cc3snc(Cc4cccc(C(F)(F)F)c4)c23)cc1]

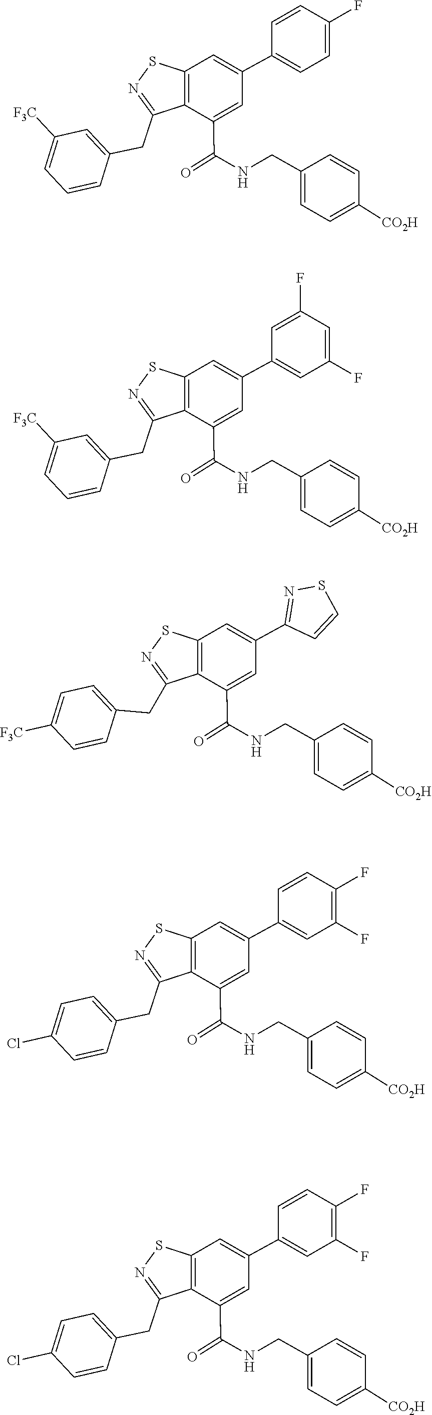 [SMILES: O=C(O)c1ccc(CNC(=O)c2cc(-c3ccc(F)cc3)cc3snc(Cc4cccc(C(F)(F)F)c4)c23)cc1 ; O=C(O)c1ccc(CNC(=O)c2cc(-c3cc(F)cc(F)c3)cc3snc(Cc4cccc(C(F)(F)F)c4)c23)cc1 ; O=C(O)c1ccc(CNC(=O)c2cc(-c3ccsn3)cc3snc(Cc4ccc(C(F)(F)F)cc4)c23)cc1 ; O=C(O)c1ccc(CNC(=O)c2cc(-c3ccc(F)c(F)c3)cc3snc(Cc4ccc(Cl)cc4)c23)cc1 ; O=C(O)c1ccc(CNC(=O)c2cc(-c3ccc(F)c(F)c3)cc3snc(Cc4ccc(Cl)cc4)c23)cc1]

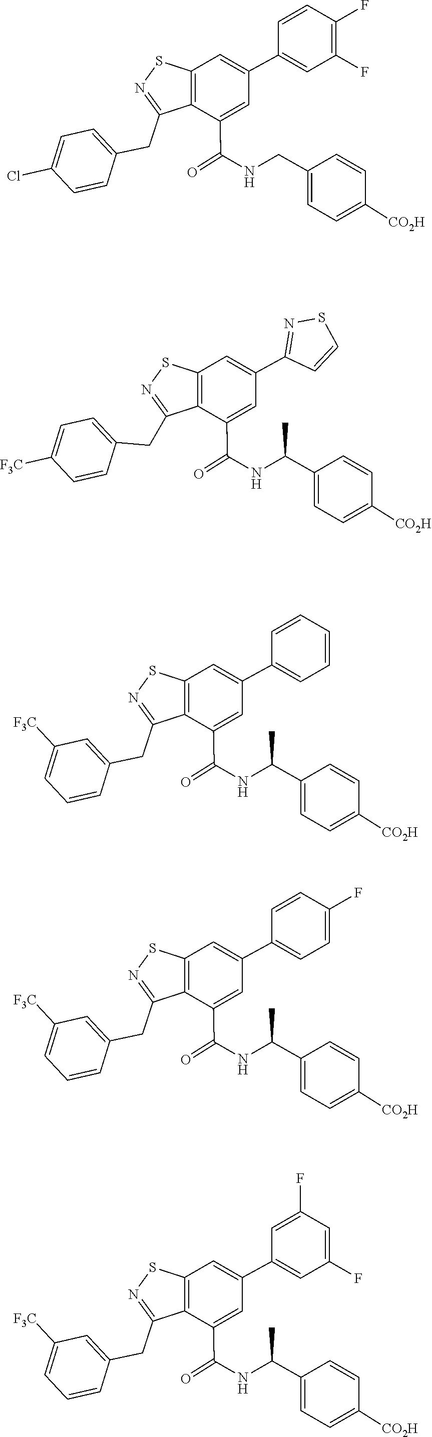 [SMILES: O=C(O)c1ccc(CNC(=O)c2cc(-c3ccc(F)c(F)c3)cc3snc(Cc4ccc(Cl)cc4)c23)cc1 ; C[C@H](NC(=O)c1cc(-c2ccsn2)cc2snc(Cc3ccc(C(F)(F)F)cc3)c12)c1ccc(C(=O)O)cc1 ; C[C@H](NC(=O)c1cc(-c2ccccc2)cc2snc(Cc3cccc(C(F)(F)F)c3)c12)c1ccc(C(=O)O)cc1 ; C[C@H](NC(=O)c1cc(-c2ccc(F)cc2)cc2snc(Cc3cccc(C(F)(F)F)c3)c12)c1ccc(C(=O)O)cc1 ; C[C@H](NC(=O)c1cc(-c2cc(F)cc(F)c2)cc2snc(Cc3cccc(C(F)(F)F)c3)c12)c1ccc(C(=O)O)cc1]

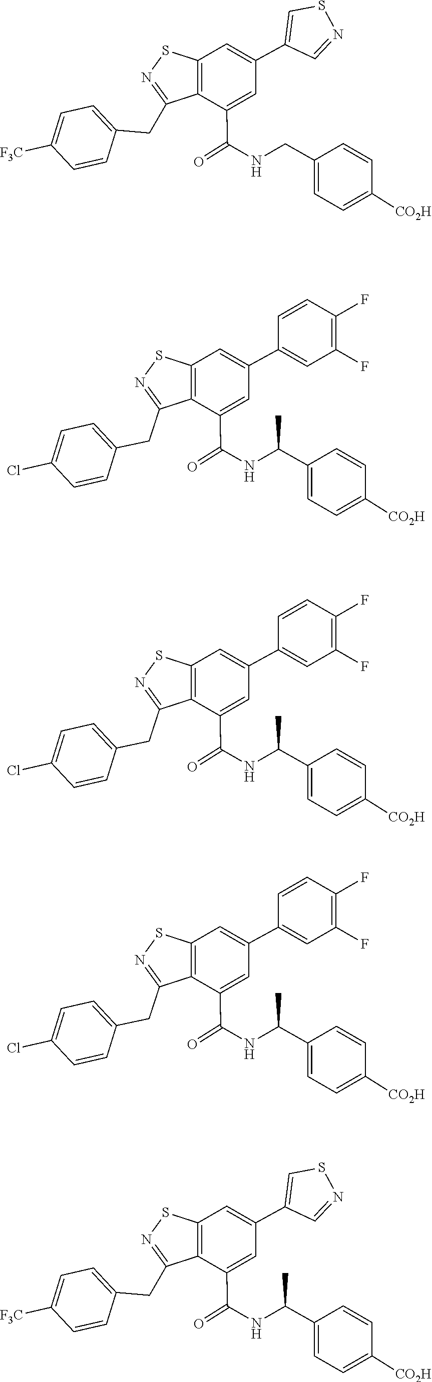 [SMILES: O=C(O)c1ccc(CNC(=O)c2cc(-c3cnsc3)cc3snc(Cc4ccc(C(F)(F)F)cc4)c23)cc1 ; C[C@H](NC(=O)c1cc(-c2ccc(F)c(F)c2)cc2snc(Cc3ccc(Cl)cc3)c12)c1ccc(C(=O)O)cc1 ; C[C@H](NC(=O)c1cc(-c2ccc(F)c(F)c2)cc2snc(Cc3ccc(Cl)cc3)c12)c1ccc(C(=O)O)cc1 ; C[C@H](NC(=O)c1cc(-c2ccc(F)c(F)c2)cc2snc(Cc3ccc(Cl)cc3)c12)c1ccc(C(=O)O)cc1 ; C[C@H](NC(=O)c1cc(-c2cnsc2)cc2snc(Cc3ccc(C(F)(F)F)cc3)c12)c1ccc(C(=O)O)cc1]

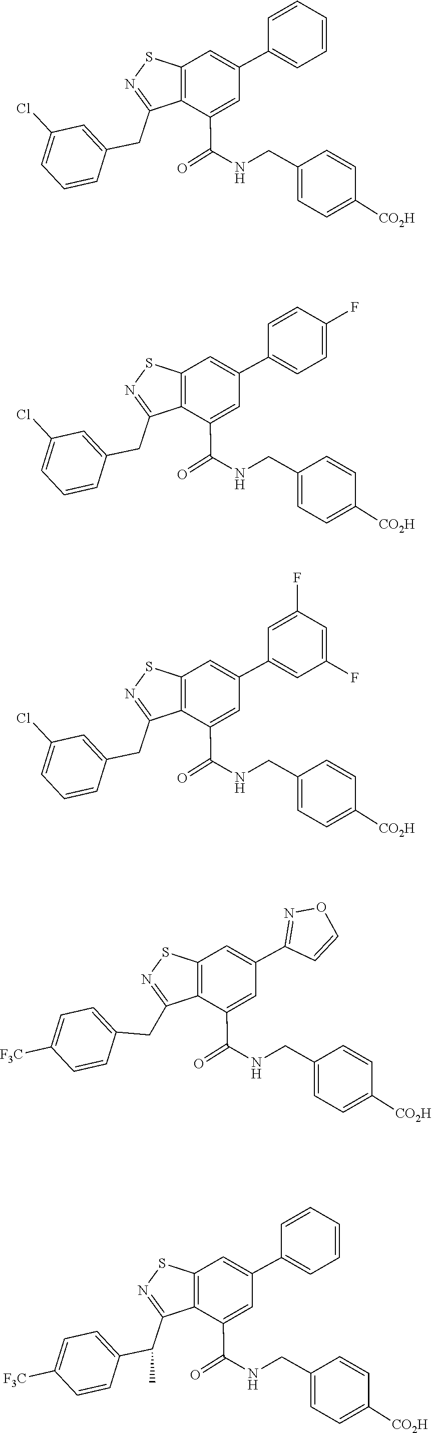 [SMILES: O=C(O)c1ccc(CNC(=O)c2cc(-c3ccccc3)cc3snc(Cc4cccc(Cl)c4)c23)cc1 ; O=C(O)c1ccc(CNC(=O)c2cc(-c3ccc(F)cc3)cc3snc(Cc4cccc(Cl)c4)c23)cc1 ; O=C(O)c1ccc(CNC(=O)c2cc(-c3cc(F)cc(F)c3)cc3snc(Cc4cccc(Cl)c4)c23)cc1 ; O=C(O)c1ccc(CNC(=O)c2cc(-c3ccon3)cc3snc(Cc4ccc(C(F)(F)F)cc4)c23)cc1 ; C[C@H](c1ccc(C(F)(F)F)cc1)c1nsc2cc(-c3ccccc3)cc(C(=O)NCc3ccc(C(=O)O)cc3)c12]

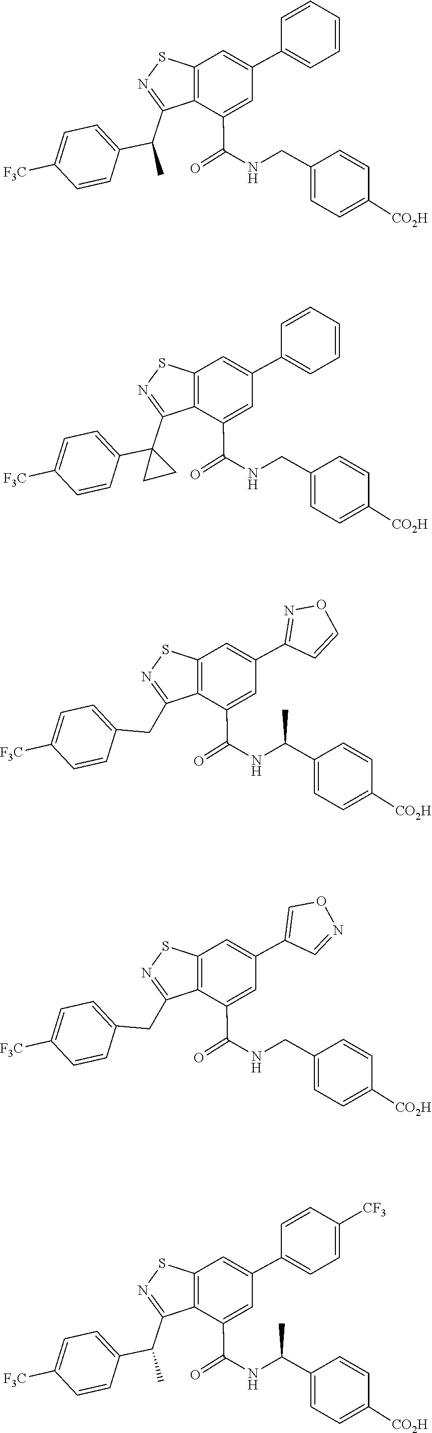 [SMILES: C[C@@H](c1ccc(C(F)(F)F)cc1)c1nsc2cc(-c3ccccc3)cc(C(=O)NCc3ccc(C(=O)O)cc3)c12 ; O=C(O)c1ccc(CNC(=O)c2cc(-c3ccccc3)cc3snc(C4(c5ccc(C(F)(F)F)cc5)CC4)c23)cc1 ; C[C@H](NC(=O)c1cc(-c2ccon2)cc2snc(Cc3ccc(C(F)(F)F)cc3)c12)c1ccc(C(=O)O)cc1 ; O=C(O)c1ccc(CNC(=O)c2cc(-c3cnoc3)cc3snc(Cc4ccc(C(F)(F)F)cc4)c23)cc1 ; C[C@H](c1ccc(C(F)(F)F)cc1)c1nsc2cc(-c3ccc(C(F)(F)F)cc3)cc(C(=O)N[C@@H](C)c3ccc(C(=O)O)cc3)c12]

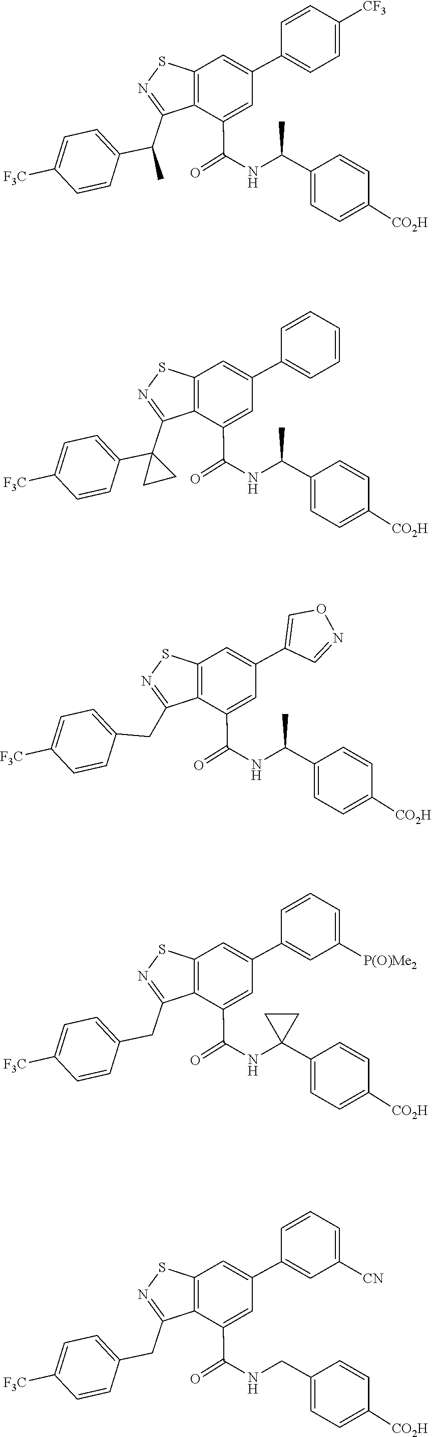

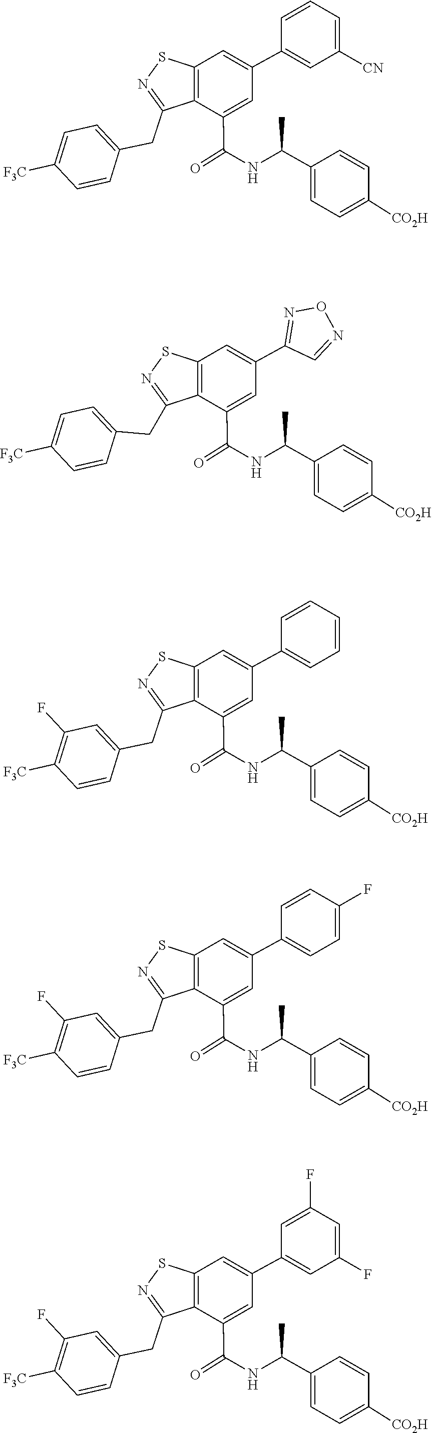 [SMILES: C[C@H](NC(=O)c1cc(-c2cccc(C#N)c2)cc2snc(Cc3ccc(C(F)(F)F)cc3)c12)c1ccc(C(=O)O)cc1 ; C[C@H](NC(=O)c1cc(-c2cnon2)cc2snc(Cc3ccc(C(F)(F)F)cc3)c12)c1ccc(C(=O)O)cc1 ; C[C@H](NC(=O)c1cc(-c2ccccc2)cc2snc(Cc3ccc(C(F)(F)F)c(F)c3)c12)c1ccc(C(=O)O)cc1 ; C[C@H](NC(=O)c1cc(-c2ccc(F)cc2)cc2snc(Cc3ccc(C(F)(F)F)c(F)c3)c12)c1ccc(C(=O)O)cc1 ; C[C@H](NC(=O)c1cc(-c2cc(F)cc(F)c2)cc2snc(Cc3ccc(C(F)(F)F)c(F)c3)c12)c1ccc(C(=O)O)cc1]

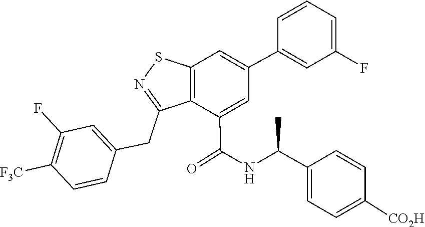 [SMILES: C[C@H](NC(=O)c1cc(-c2cccc(F)c2)cc2snc(Cc3ccc(C(F)(F)F)c(F)c3)c12)c1ccc(C(=O)O)cc1]

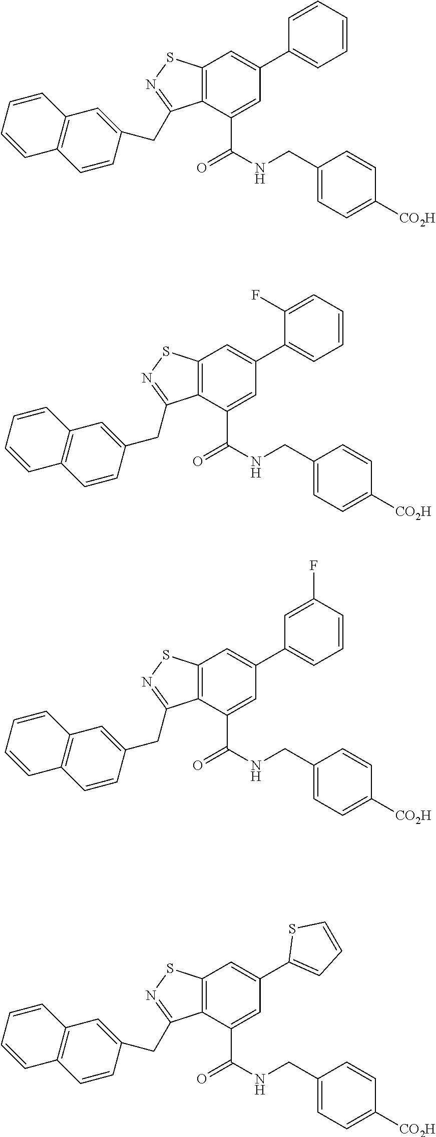 [SMILES: O=C(O)c1ccc(CNC(=O)c2cc(-c3ccccc3)cc3snc(Cc4ccc5ccccc5c4)c23)cc1 ; O=C(O)c1ccc(CNC(=O)c2cc(-c3ccccc3F)cc3snc(Cc4ccc5ccccc5c4)c23)cc1 ; O=C(O)c1ccc(CNC(=O)c2cc(-c3cccc(F)c3)cc3snc(Cc4ccc5ccccc5c4)c23)cc1 ; O=C(O)c1ccc(CNC(=O)c2cc(-c3cccs3)cc3snc(Cc4ccc5ccccc5c4)c23)cc1]

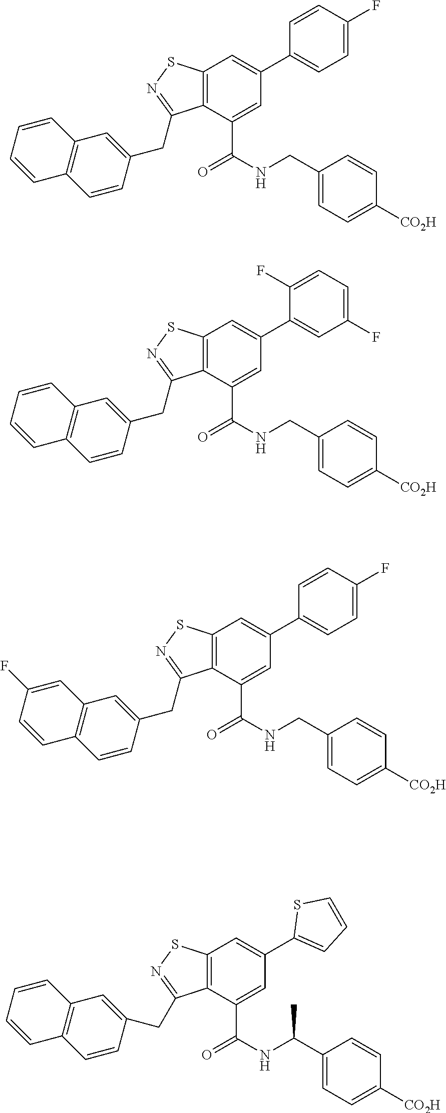 [SMILES: O=C(O)c1ccc(CNC(=O)c2cc(-c3ccc(F)cc3)cc3snc(Cc4ccc5ccccc5c4)c23)cc1 ; O=C(O)c1ccc(CNC(=O)c2cc(-c3cc(F)ccc3F)cc3snc(Cc4ccc5ccccc5c4)c23)cc1 ; O=C(O)c1ccc(CNC(=O)c2cc(-c3ccc(F)cc3)cc3snc(Cc4ccc5ccc(F)cc5c4)c23)cc1 ; C[C@H](NC(=O)c1cc(-c2cccs2)cc2snc(Cc3ccc4ccccc4c3)c12)c1ccc(C(=O)O)cc1]

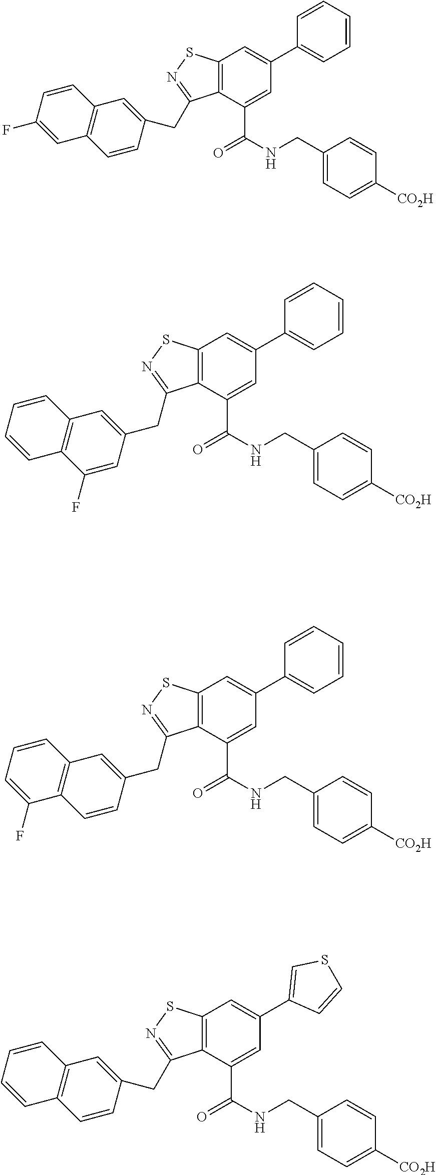 [SMILES: O=C(O)c1ccc(CNC(=O)c2cc(-c3ccccc3)cc3snc(Cc4ccc5cc(F)ccc5c4)c23)cc1 ; O=C(O)c1ccc(CNC(=O)c2cc(-c3ccccc3)cc3snc(Cc4cc(F)c5ccccc5c4)c23)cc1 ; O=C(O)c1ccc(CNC(=O)c2cc(-c3ccccc3)cc3snc(Cc4ccc5c(F)cccc5c4)c23)cc1 ; O=C(O)c1ccc(CNC(=O)c2cc(-c3ccsc3)cc3snc(Cc4ccc5ccccc5c4)c23)cc1]

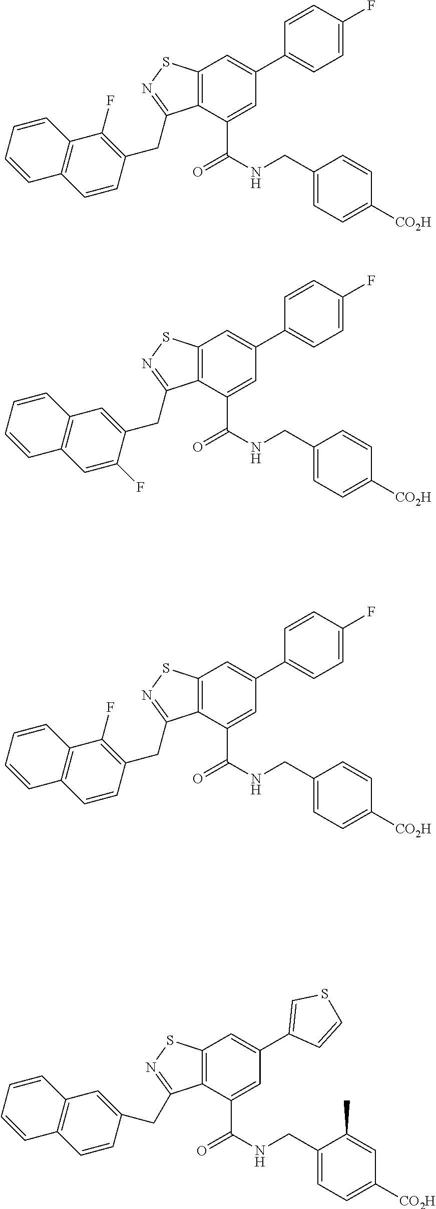

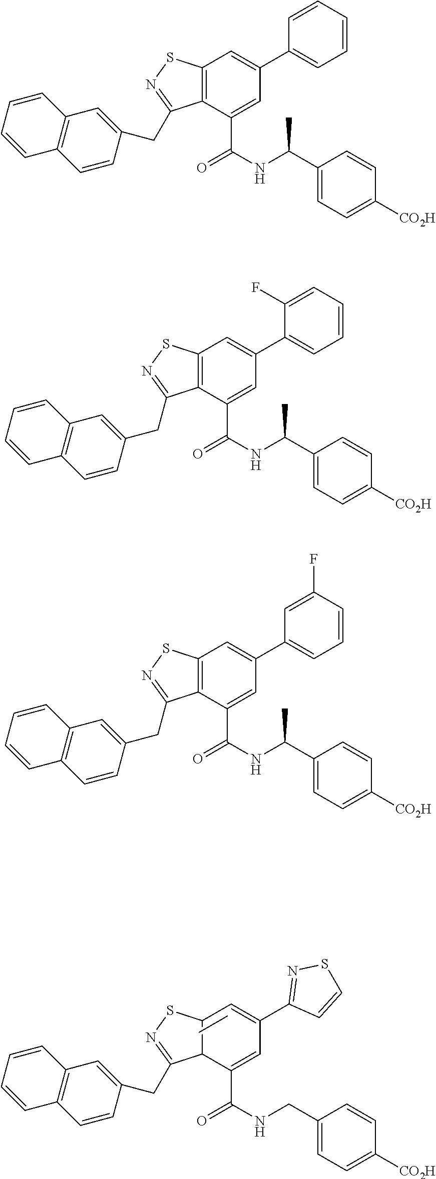

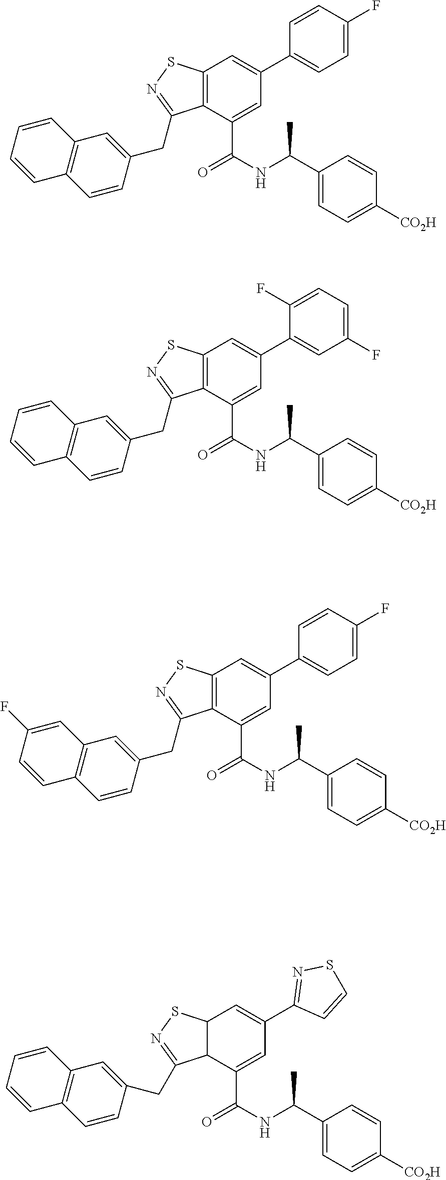 [SMILES: C[C@H](NC(=O)c1cc(-c2ccc(F)cc2)cc2snc(Cc3ccc4ccccc4c3)c12)c1ccc(C(=O)O)cc1 ; C[C@H](NC(=O)c1cc(-c2cc(F)ccc2F)cc2snc(Cc3ccc4ccccc4c3)c12)c1ccc(C(=O)O)cc1 ; C[C@H](NC(=O)c1cc(-c2ccc(F)cc2)cc2snc(Cc3ccc4ccc(F)cc4c3)c12)c1ccc(C(=O)O)cc1 ; C[C@H](NC(=O)C1=CC(c2ccsn2)=CC2SN=C(Cc3ccc4ccccc4c3)C12)c1ccc(C(=O)O)cc1]

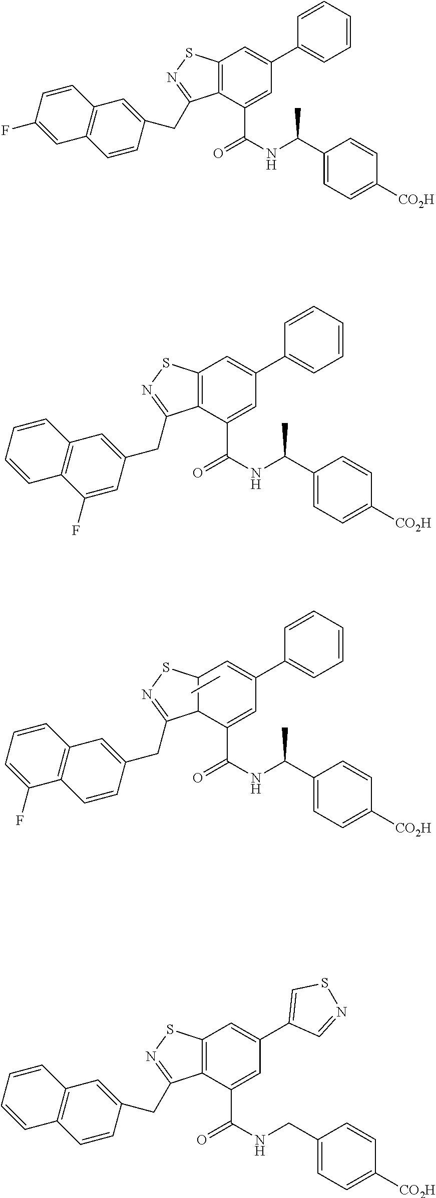

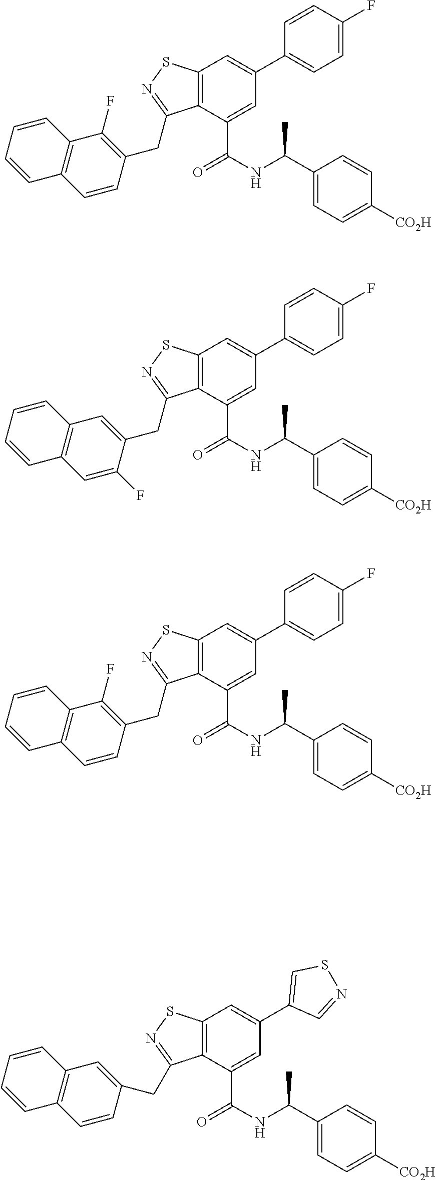 [SMILES: C[C@H](NC(=O)c1cc(-c2ccc(F)cc2)cc2snc(Cc3ccc4ccccc4c3F)c12)c1ccc(C(=O)O)cc1 ; C[C@H](NC(=O)c1cc(-c2ccc(F)cc2)cc2snc(Cc3cc4ccccc4cc3F)c12)c1ccc(C(=O)O)cc1 ; C[C@H](NC(=O)c1cc(-c2ccc(F)cc2)cc2snc(Cc3ccc4ccccc4c3F)c12)c1ccc(C(=O)O)cc1 ; C[C@H](NC(=O)c1cc(-c2cnsc2)cc2snc(Cc3ccc4ccccc4c3)c12)c1ccc(C(=O)O)cc1]

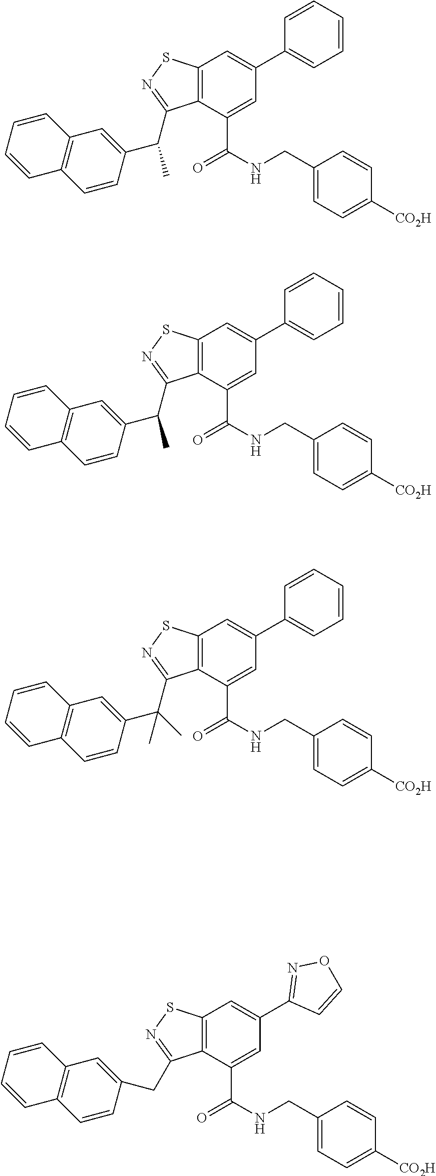 [SMILES: C[C@H](c1ccc2ccccc2c1)c1nsc2cc(-c3ccccc3)cc(C(=O)NCc3ccc(C(=O)O)cc3)c12 ; C[C@@H](c1ccc2ccccc2c1)c1nsc2cc(-c3ccccc3)cc(C(=O)NCc3ccc(C(=O)O)cc3)c12 ; CC(C)(c1ccc2ccccc2c1)c1nsc2cc(-c3ccccc3)cc(C(=O)NCc3ccc(C(=O)O)cc3)c12 ; O=C(O)c1ccc(CNC(=O)c2cc(-c3ccon3)cc3snc(Cc4ccc5ccccc5c4)c23)cc1]

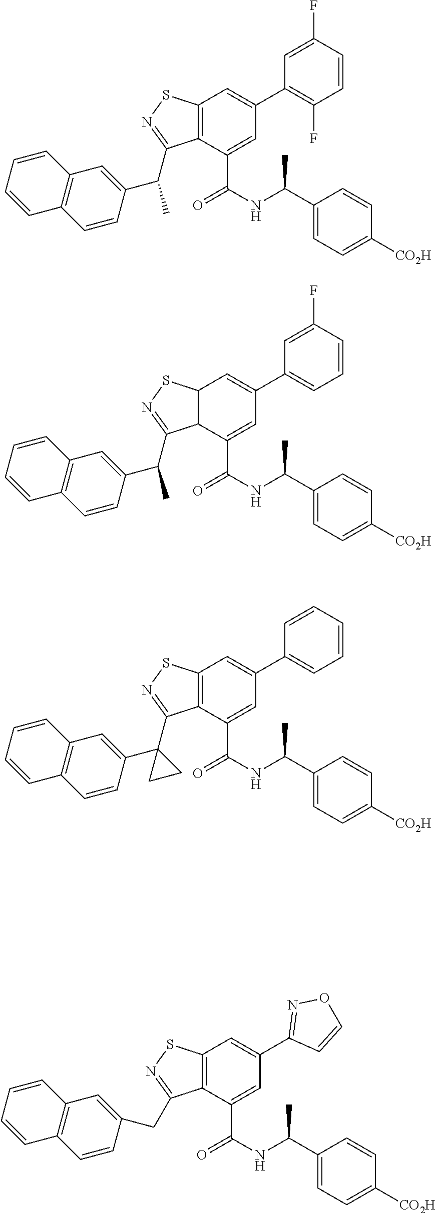

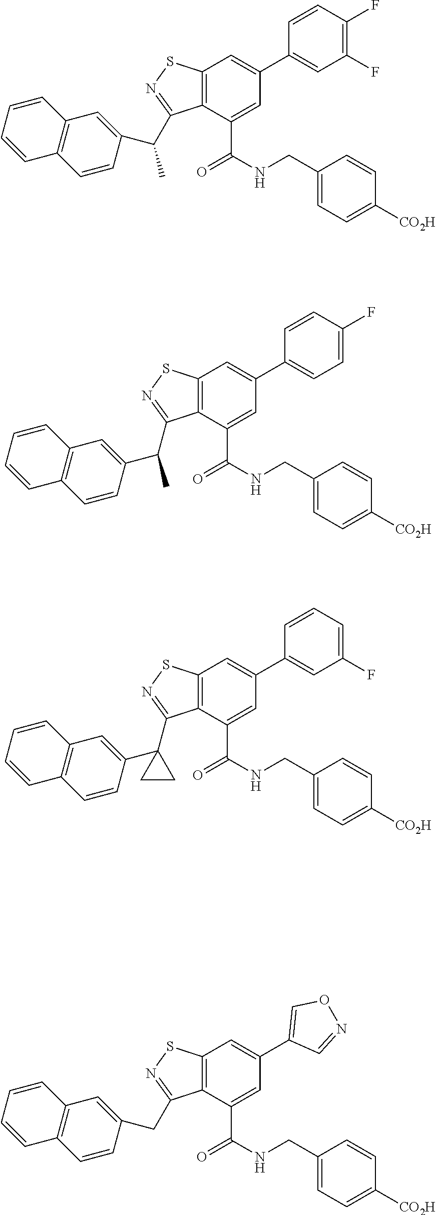 [SMILES: C[C@H](c1ccc2ccccc2c1)c1nsc2cc(-c3ccc(F)c(F)c3)cc(C(=O)NCc3ccc(C(=O)O)cc3)c12 ; C[C@@H](c1ccc2ccccc2c1)c1nsc2cc(-c3ccc(F)cc3)cc(C(=O)NCc3ccc(C(=O)O)cc3)c12 ; O=C(O)c1ccc(CNC(=O)c2cc(-c3cccc(F)c3)cc3snc(C4(c5ccc6ccccc6c5)CC4)c23)cc1 ; O=C(O)c1ccc(CNC(=O)c2cc(-c3cnoc3)cc3snc(Cc4ccc5ccccc5c4)c23)cc1]

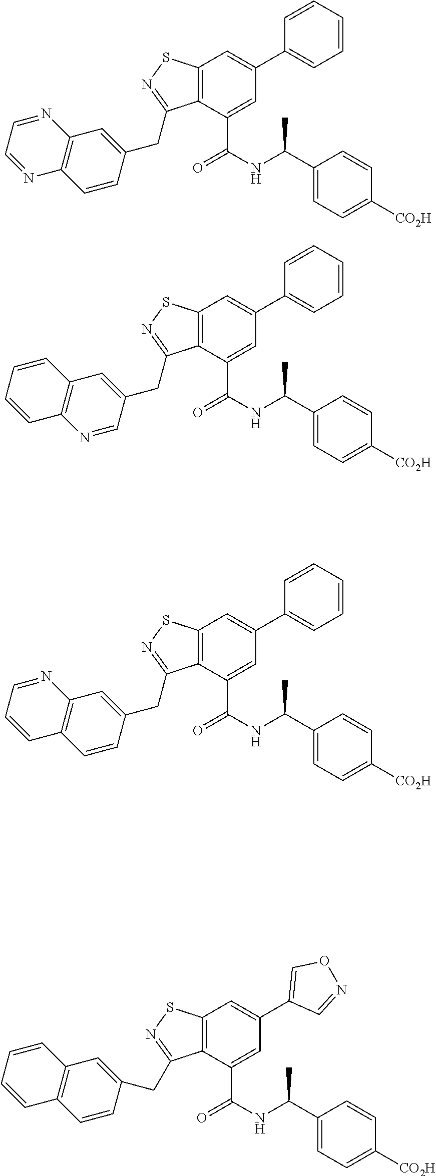

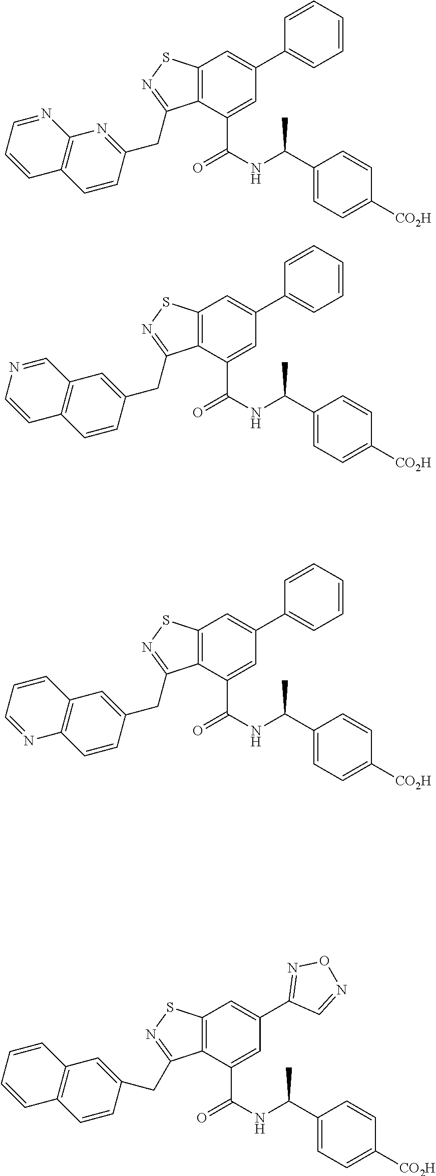

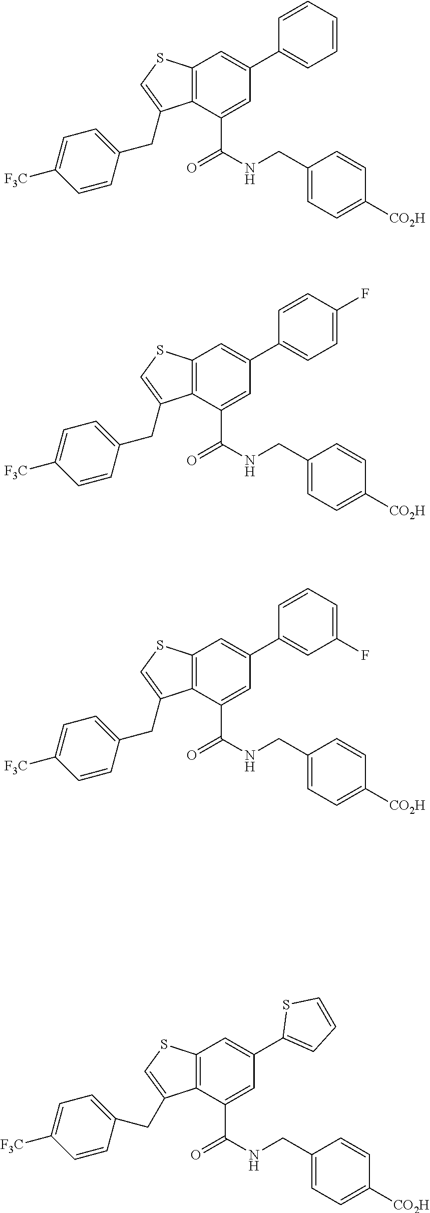 [SMILES: O=C(O)c1ccc(CNC(=O)c2cc(-c3ccccc3)cc3scc(Cc4ccc(C(F)(F)F)cc4)c23)cc1 ; O=C(O)c1ccc(CNC(=O)c2cc(-c3ccc(F)cc3)cc3scc(Cc4ccc(C(F)(F)F)cc4)c23)cc1 ; O=C(O)c1ccc(CNC(=O)c2cc(-c3cccc(F)c3)cc3scc(Cc4ccc(C(F)(F)F)cc4)c23)cc1 ; O=C(O)c1ccc(CNC(=O)c2cc(-c3cccs3)cc3scc(Cc4ccc(C(F)(F)F)cc4)c23)cc1]

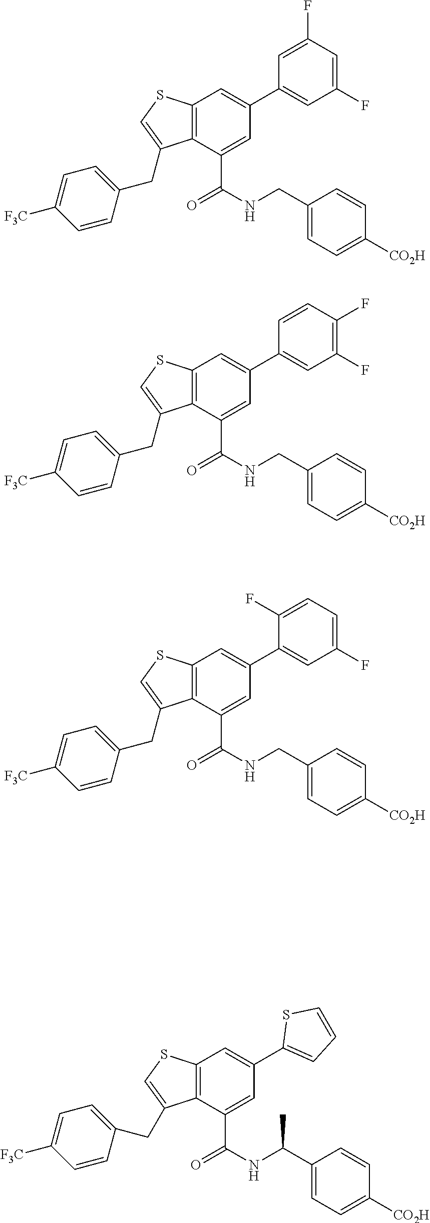 [SMILES: O=C(O)c1ccc(CNC(=O)c2cc(-c3cc(F)cc(F)c3)cc3scc(Cc4ccc(C(F)(F)F)cc4)c23)cc1 ; O=C(O)c1ccc(CNC(=O)c2cc(-c3ccc(F)c(F)c3)cc3scc(Cc4ccc(C(F)(F)F)cc4)c23)cc1 ; O=C(O)c1ccc(CNC(=O)c2cc(-c3cc(F)ccc3F)cc3scc(Cc4ccc(C(F)(F)F)cc4)c23)cc1 ; C[C@H](NC(=O)c1cc(-c2cccs2)cc2scc(Cc3ccc(C(F)(F)F)cc3)c12)c1ccc(C(=O)O)cc1]

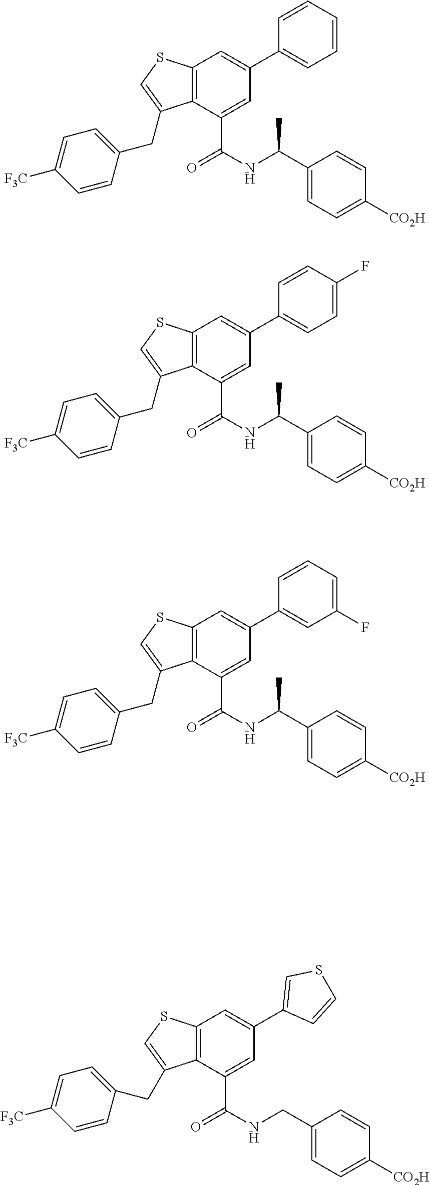 [SMILES: C[C@H](NC(=O)c1cc(-c2ccccc2)cc2scc(Cc3ccc(C(F)(F)F)cc3)c12)c1ccc(C(=O)O)cc1 ; C[C@H](NC(=O)c1cc(-c2ccc(F)cc2)cc2scc(Cc3ccc(C(F)(F)F)cc3)c12)c1ccc(C(=O)O)cc1 ; C[C@H](NC(=O)c1cc(-c2cccc(F)c2)cc2scc(Cc3ccc(C(F)(F)F)cc3)c12)c1ccc(C(=O)O)cc1 ; O=C(O)c1ccc(CNC(=O)c2cc(-c3ccsc3)cc3scc(Cc4ccc(C(F)(F)F)cc4)c23)cc1]

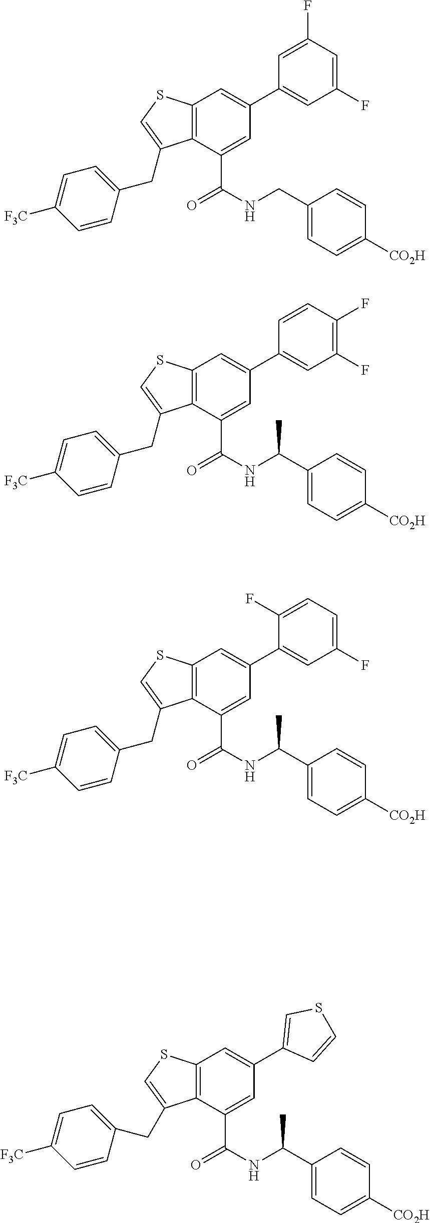 [SMILES: O=C(O)c1ccc(CNC(=O)c2cc(-c3cc(F)cc(F)c3)cc3scc(Cc4ccc(C(F)(F)F)cc4)c23)cc1 ; C[C@H](NC(=O)c1cc(-c2ccc(F)c(F)c2)cc2scc(Cc3ccc(C(F)(F)F)cc3)c12)c1ccc(C(=O)O)cc1 ; C[C@H](NC(=O)c1cc(-c2cc(F)ccc2F)cc2scc(Cc3ccc(C(F)(F)F)cc3)c12)c1ccc(C(=O)O)cc1 ; C[C@H](NC(=O)c1cc(-c2ccsc2)cc2scc(Cc3ccc(C(F)(F)F)cc3)c12)c1ccc(C(=O)O)cc1]

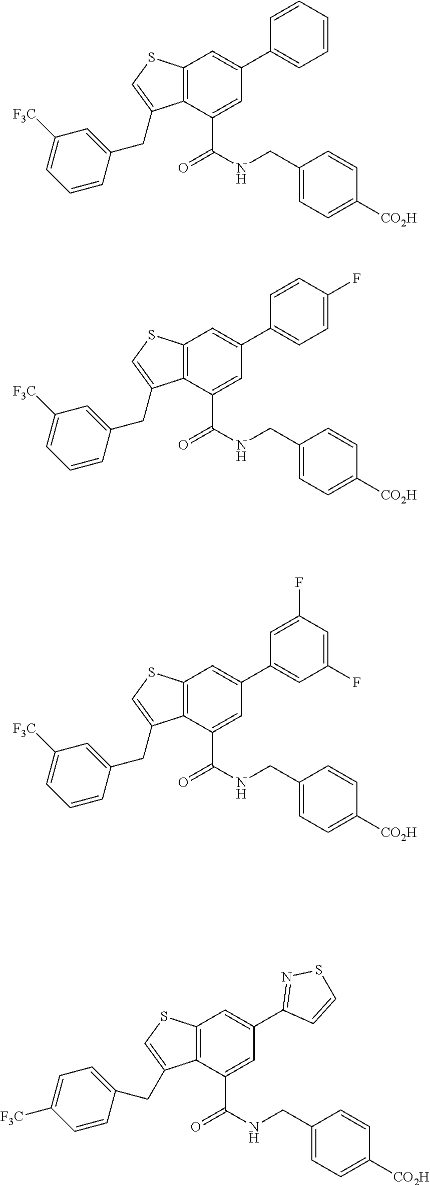 [SMILES: O=C(O)c1ccc(CNC(=O)c2cc(-c3ccccc3)cc3scc(Cc4cccc(C(F)(F)F)c4)c23)cc1 ; O=C(O)c1ccc(CNC(=O)c2cc(-c3ccc(F)cc3)cc3scc(Cc4cccc(C(F)(F)F)c4)c23)cc1 ; O=C(O)c1ccc(CNC(=O)c2cc(-c3cc(F)cc(F)c3)cc3scc(Cc4cccc(C(F)(F)F)c4)c23)cc1 ; O=C(O)c1ccc(CNC(=O)c2cc(-c3ccsn3)cc3scc(Cc4ccc(C(F)(F)F)cc4)c23)cc1]

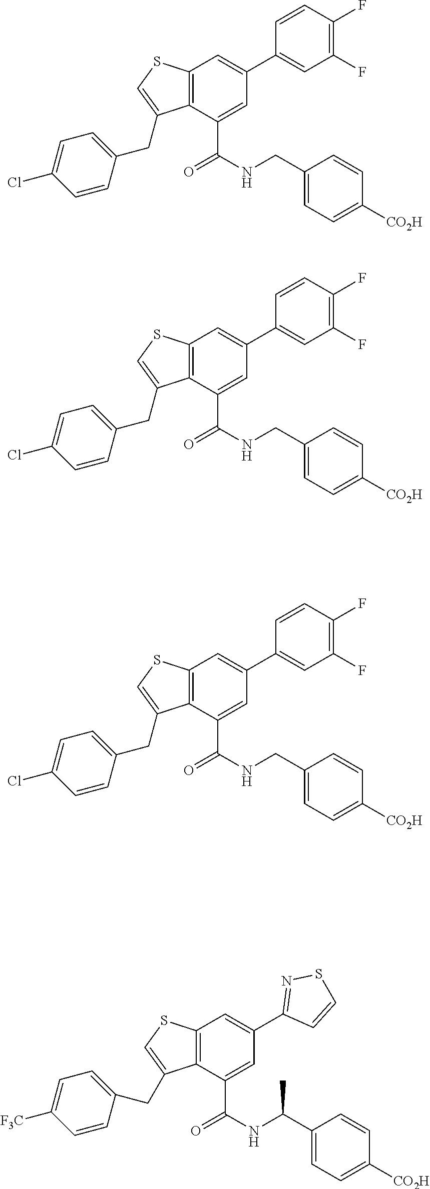 [SMILES: O=C(O)c1ccc(CNC(=O)c2cc(-c3ccc(F)c(F)c3)cc3scc(Cc4ccc(Cl)cc4)c23)cc1 ; O=C(O)c1ccc(CNC(=O)c2cc(-c3ccc(F)c(F)c3)cc3scc(Cc4ccc(Cl)cc4)c23)cc1 ; O=C(O)c1ccc(CNC(=O)c2cc(-c3ccc(F)c(F)c3)cc3scc(Cc4ccc(Cl)cc4)c23)cc1 ; C[C@H](NC(=O)c1cc(-c2ccsn2)cc2scc(Cc3ccc(C(F)(F)F)cc3)c12)c1ccc(C(=O)O)cc1]

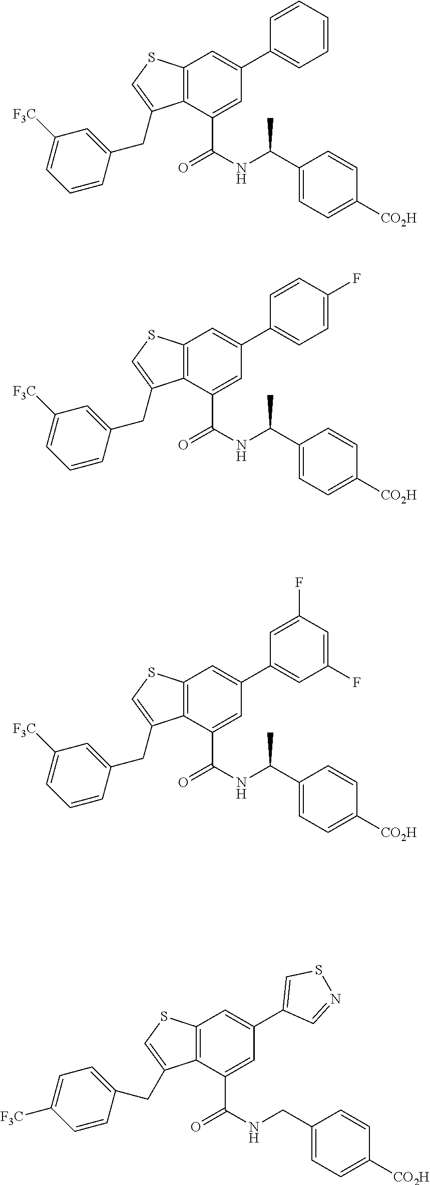 [SMILES: C[C@H](NC(=O)c1cc(-c2ccccc2)cc2scc(Cc3cccc(C(F)(F)F)c3)c12)c1ccc(C(=O)O)cc1 ; C[C@H](NC(=O)c1cc(-c2ccc(F)cc2)cc2scc(Cc3cccc(C(F)(F)F)c3)c12)c1ccc(C(=O)O)cc1 ; C[C@H](NC(=O)c1cc(-c2cc(F)cc(F)c2)cc2scc(Cc3cccc(C(F)(F)F)c3)c12)c1ccc(C(=O)O)cc1 ; O=C(O)c1ccc(CNC(=O)c2cc(-c3cnsc3)cc3scc(Cc4ccc(C(F)(F)F)cc4)c23)cc1]

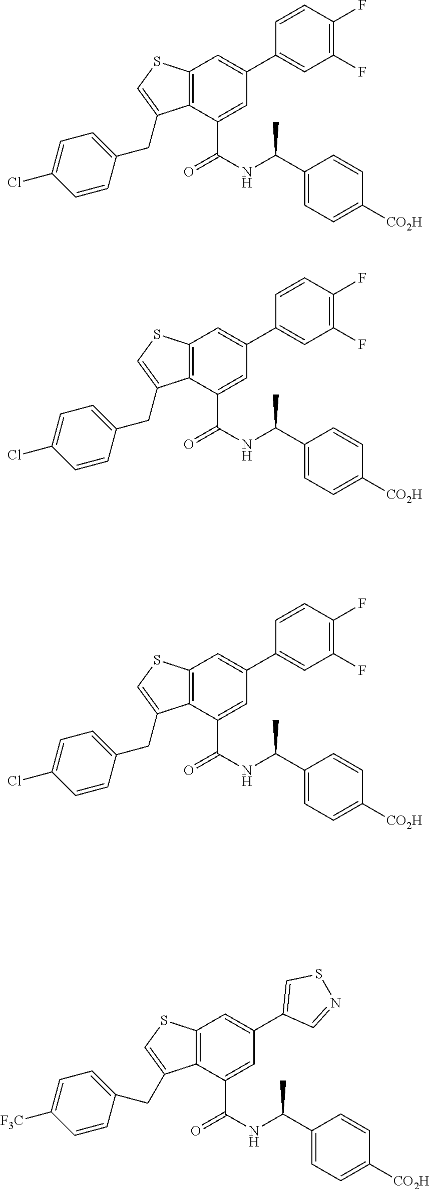 [SMILES: C[C@H](NC(=O)c1cc(-c2ccc(F)c(F)c2)cc2scc(Cc3ccc(Cl)cc3)c12)c1ccc(C(=O)O)cc1 ; C[C@H](NC(=O)c1cc(-c2ccc(F)c(F)c2)cc2scc(Cc3ccc(Cl)cc3)c12)c1ccc(C(=O)O)cc1 ; C[C@H](NC(=O)c1cc(-c2ccc(F)c(F)c2)cc2scc(Cc3ccc(Cl)cc3)c12)c1ccc(C(=O)O)cc1 ; C[C@H](NC(=O)c1cc(-c2cnsc2)cc2scc(Cc3ccc(C(F)(F)F)cc3)c12)c1ccc(C(=O)O)cc1]

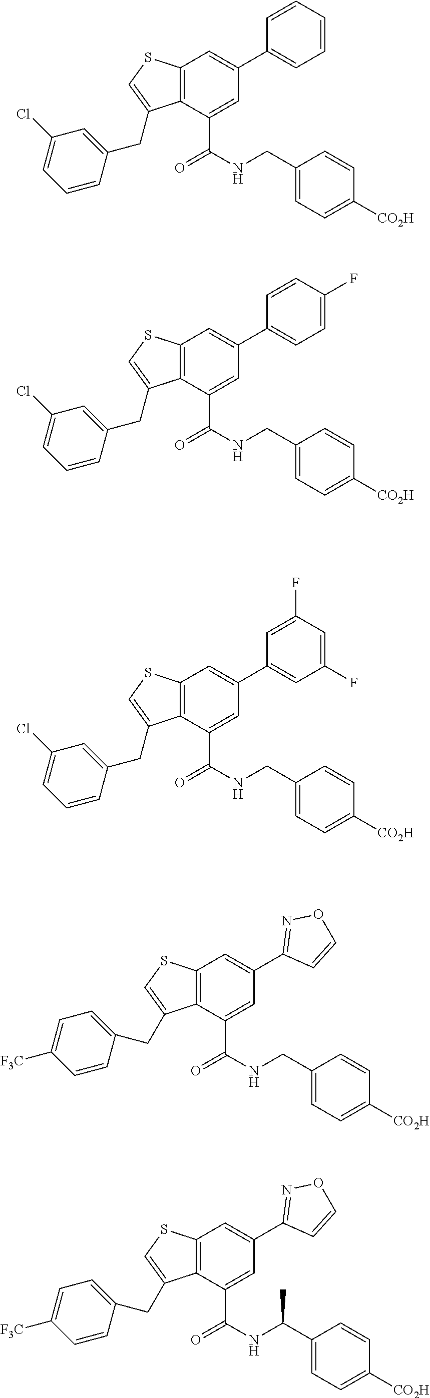 [SMILES: O=C(O)c1ccc(CNC(=O)c2cc(-c3ccccc3)cc3scc(Cc4cccc(Cl)c4)c23)cc1 ; O=C(O)c1ccc(CNC(=O)c2cc(-c3ccc(F)cc3)cc3scc(Cc4cccc(Cl)c4)c23)cc1 ; O=C(O)c1ccc(CNC(=O)c2cc(-c3cc(F)cc(F)c3)cc3scc(Cc4cccc(Cl)c4)c23)cc1 ; O=C(O)c1ccc(CNC(=O)c2cc(-c3ccon3)cc3scc(Cc4ccc(C(F)(F)F)cc4)c23)cc1 ; C[C@H](NC(=O)c1cc(-c2ccon2)cc2scc(Cc3ccc(C(F)(F)F)cc3)c12)c1ccc(C(=O)O)cc1]

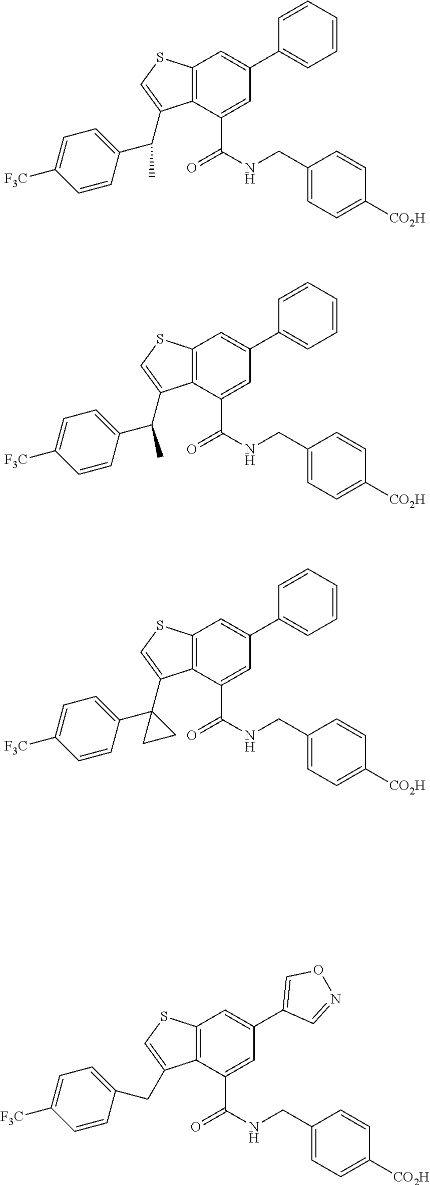 [SMILES: C[C@H](c1ccc(C(F)(F)F)cc1)c1csc2cc(-c3ccccc3)cc(C(=O)NCc3ccc(C(=O)O)cc3)c12 ; C[C@@H](c1ccc(C(F)(F)F)cc1)c1csc2cc(-c3ccccc3)cc(C(=O)NCc3ccc(C(=O)O)cc3)c12 ; O=C(O)c1ccc(CNC(=O)c2cc(-c3ccccc3)cc3scc(C4(c5ccc(C(F)(F)F)cc5)CC4)c23)cc1 ; O=C(O)c1ccc(CNC(=O)c2cc(-c3cnoc3)cc3scc(Cc4ccc(C(F)(F)F)cc4)c23)cc1]

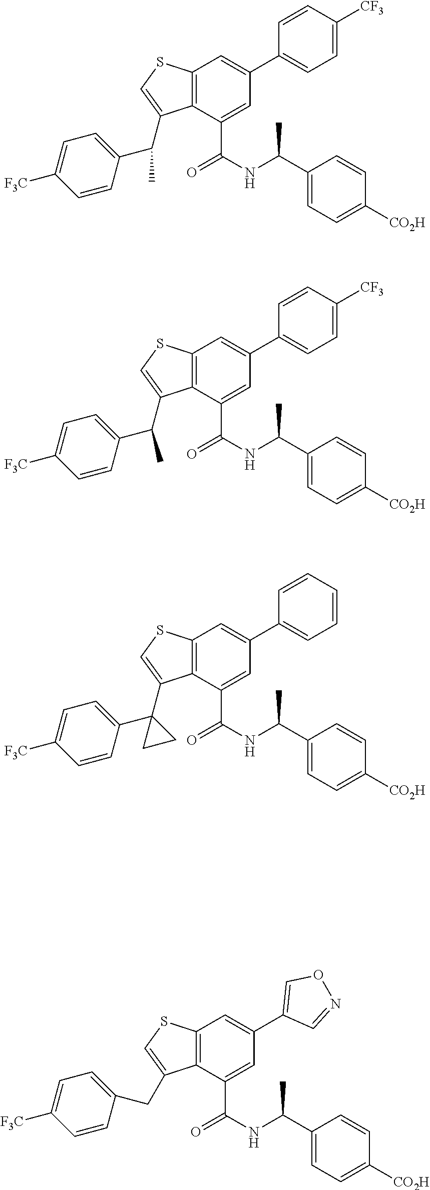 [SMILES: C[C@H](c1ccc(C(F)(F)F)cc1)c1csc2cc(-c3ccc(C(F)(F)F)cc3)cc(C(=O)N[C@@H](C)c3ccc(C(=O)O)cc3)c12 ; C[C@@H](c1ccc(C(F)(F)F)cc1)c1csc2cc(-c3ccc(C(F)(F)F)cc3)cc(C(=O)N[C@@H](C)c3ccc(C(=O)O)cc3)c12 ; C[C@H](NC(=O)c1cc(-c2ccccc2)cc2scc(C3(c4ccc(C(F)(F)F)cc4)CC3)c12)c1ccc(C(=O)O)cc1 ; C[C@H](NC(=O)c1cc(-c2cnoc2)cc2scc(Cc3ccc(C(F)(F)F)cc3)c12)c1ccc(C(=O)O)cc1]

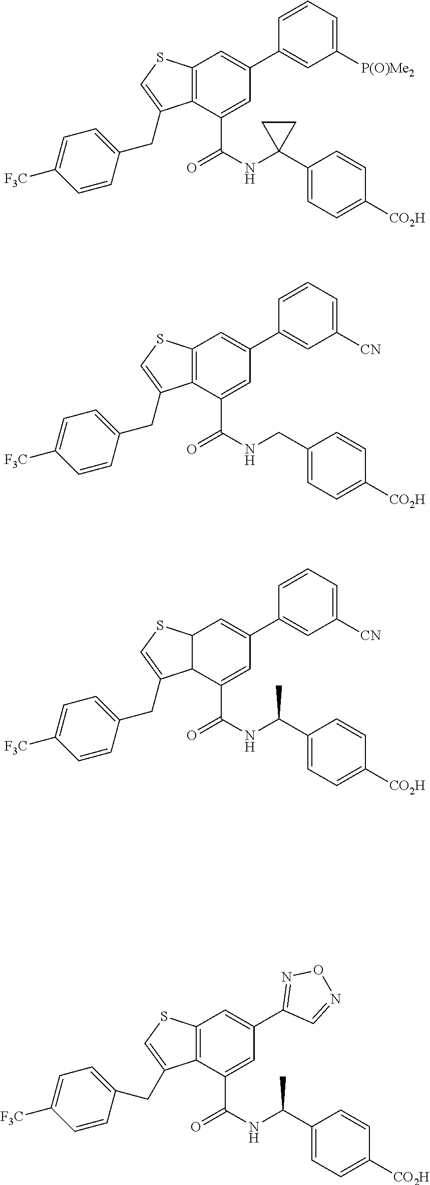

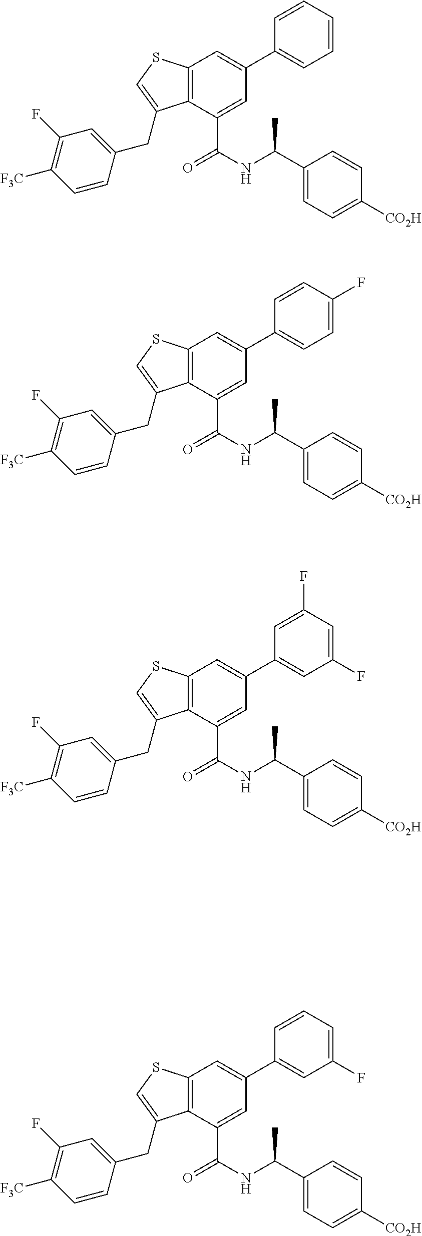 [SMILES: C[C@H](NC(=O)c1cc(-c2ccccc2)cc2scc(Cc3ccc(C(F)(F)F)c(F)c3)c12)c1ccc(C(=O)O)cc1 ; C[C@H](NC(=O)c1cc(-c2ccc(F)cc2)cc2scc(Cc3ccc(C(F)(F)F)c(F)c3)c12)c1ccc(C(=O)O)cc1 ; C[C@H](NC(=O)c1cc(-c2cc(F)cc(F)c2)cc2scc(Cc3ccc(C(F)(F)F)c(F)c3)c12)c1ccc(C(=O)O)cc1 ; C[C@H](NC(=O)c1cc(-c2cccc(F)c2)cc2scc(Cc3ccc(C(F)(F)F)c(F)c3)c12)c1ccc(C(=O)O)cc1]

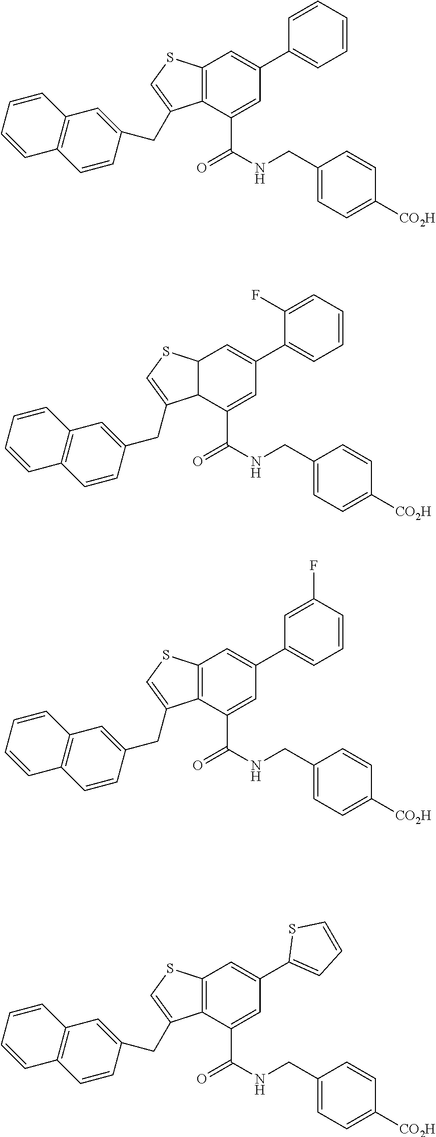 [SMILES: O=C(O)c1ccc(CNC(=O)c2cc(-c3ccccc3)cc3scc(Cc4ccc5ccccc5c4)c23)cc1 ; O=C(NCc1ccc(C(=O)O)cc1)C1=CC(c2ccccc2F)=CC2SC=C(Cc3ccc4ccccc4c3)C12 ; O=C(O)c1ccc(CNC(=O)c2cc(-c3cccc(F)c3)cc3scc(Cc4ccc5ccccc5c4)c23)cc1 ; O=C(O)c1ccc(CNC(=O)c2cc(-c3cccs3)cc3scc(Cc4ccc5ccccc5c4)c23)cc1]

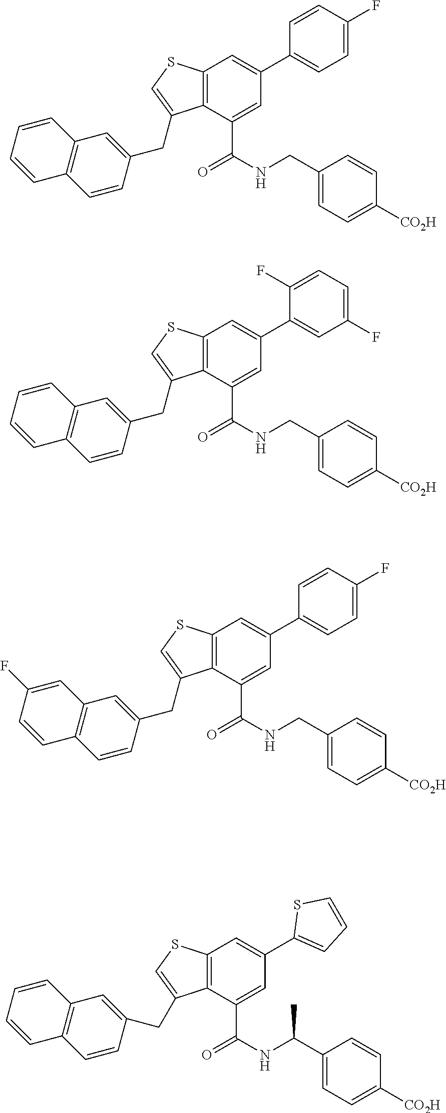 [SMILES: O=C(O)c1ccc(CNC(=O)c2cc(-c3ccc(F)cc3)cc3scc(Cc4ccc5ccccc5c4)c23)cc1 ; O=C(O)c1ccc(CNC(=O)c2cc(-c3cc(F)ccc3F)cc3scc(Cc4ccc5ccccc5c4)c23)cc1 ; O=C(O)c1ccc(CNC(=O)c2cc(-c3ccc(F)cc3)cc3scc(Cc4ccc5ccc(F)cc5c4)c23)cc1 ; C[C@H](NC(=O)c1cc(-c2cccs2)cc2scc(Cc3ccc4ccccc4c3)c12)c1ccc(C(=O)O)cc1]

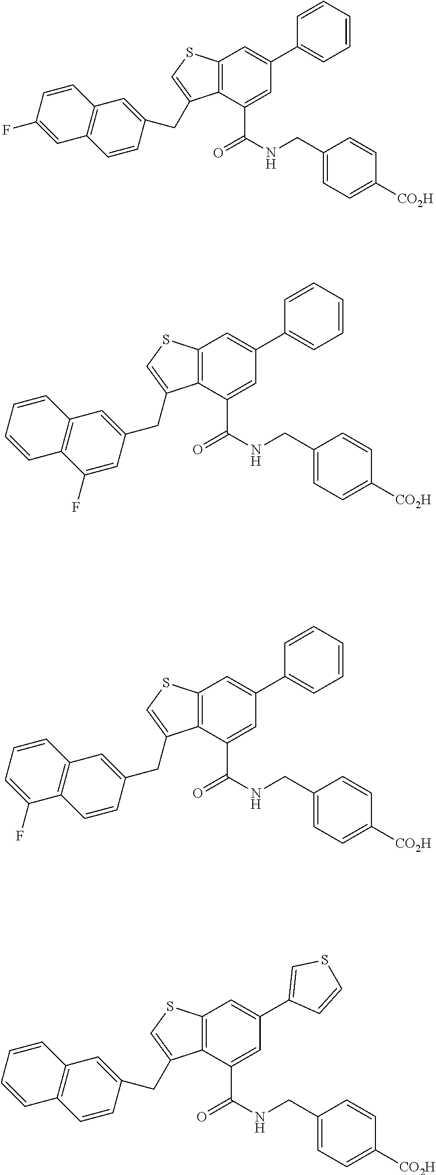 [SMILES: O=C(O)c1ccc(CNC(=O)c2cc(-c3ccccc3)cc3scc(Cc4ccc5cc(F)ccc5c4)c23)cc1 ; O=C(O)c1ccc(CNC(=O)c2cc(-c3ccccc3)cc3scc(Cc4cc(F)c5ccccc5c4)c23)cc1 ; O=C(O)c1ccc(CNC(=O)c2cc(-c3ccccc3)cc3scc(Cc4ccc5c(F)cccc5c4)c23)cc1 ; O=C(O)c1ccc(CNC(=O)c2cc(-c3ccsc3)cc3scc(Cc4ccc5ccccc5c4)c23)cc1]

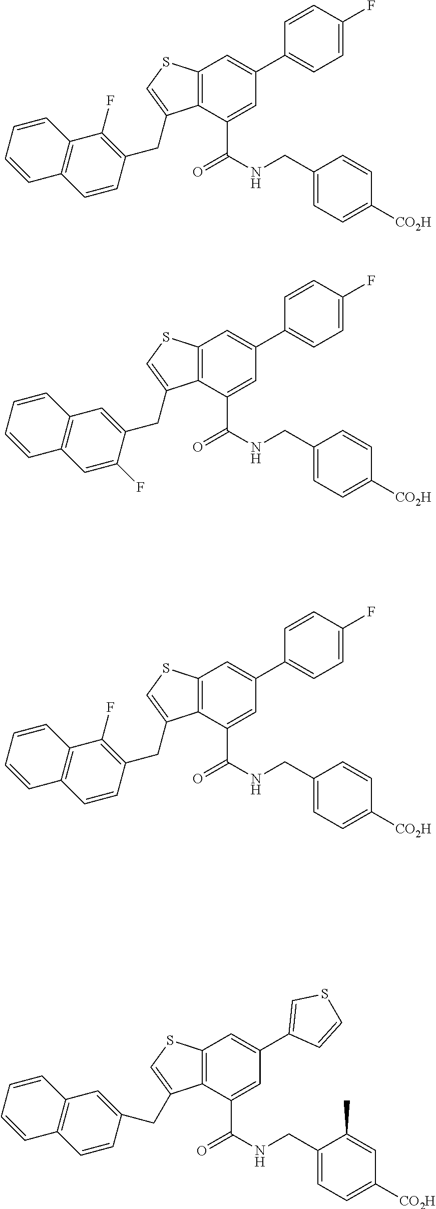

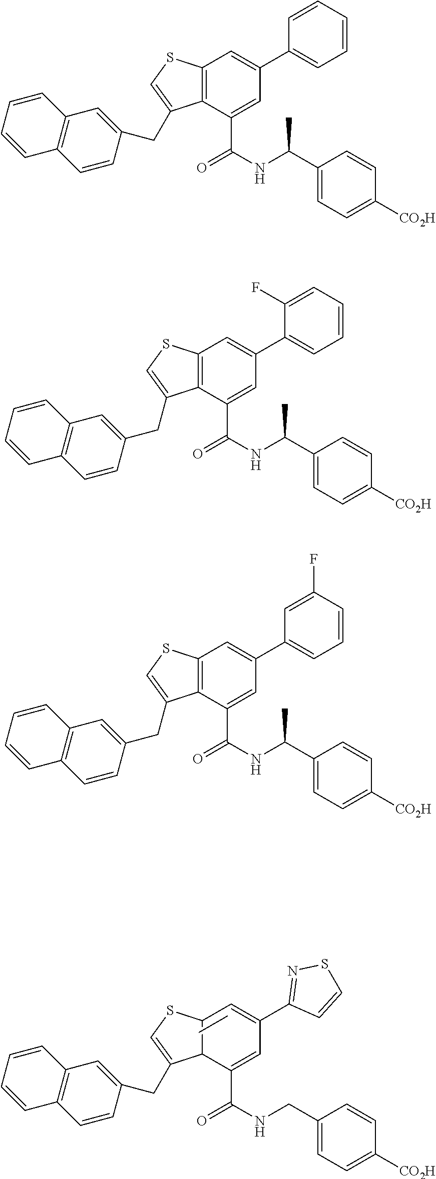

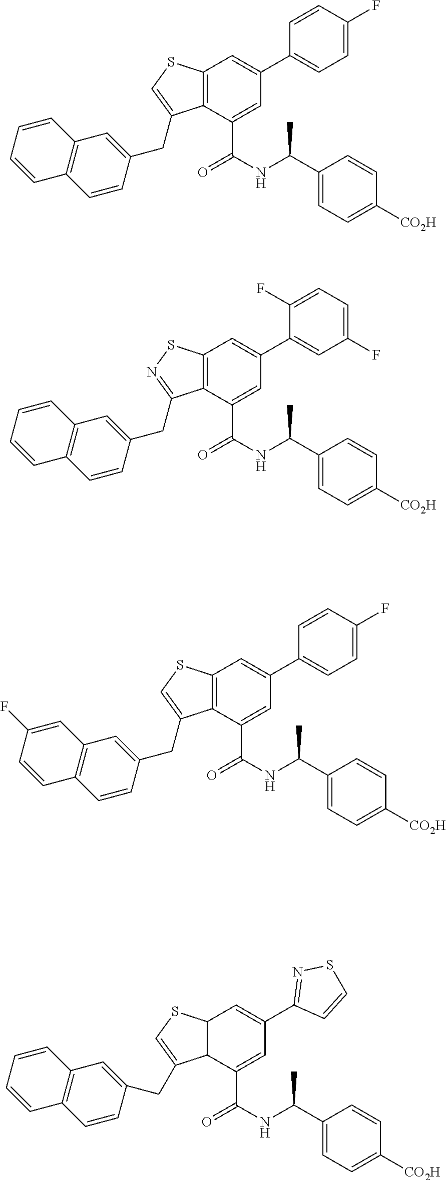 [SMILES: C[C@H](NC(=O)c1cc(-c2ccc(F)cc2)cc2scc(Cc3ccc4ccccc4c3)c12)c1ccc(C(=O)O)cc1 ; C[C@H](NC(=O)c1cc(-c2cc(F)ccc2F)cc2snc(Cc3ccc4ccccc4c3)c12)c1ccc(C(=O)O)cc1 ; C[C@H](NC(=O)c1cc(-c2ccc(F)cc2)cc2scc(Cc3ccc4ccc(F)cc4c3)c12)c1ccc(C(=O)O)cc1 ; C[C@H](NC(=O)C1=CC(c2ccsn2)=CC2SC=C(Cc3ccc4ccccc4c3)C12)c1ccc(C(=O)O)cc1]

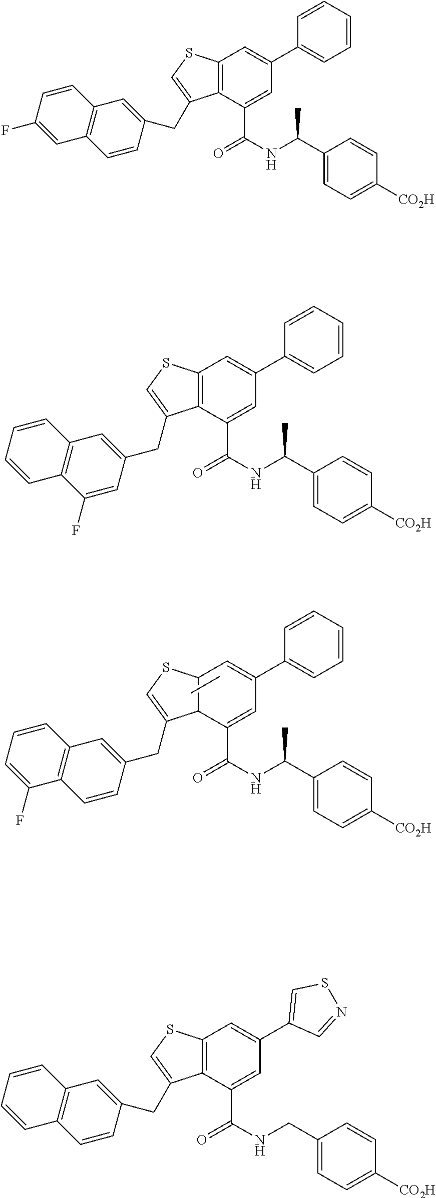

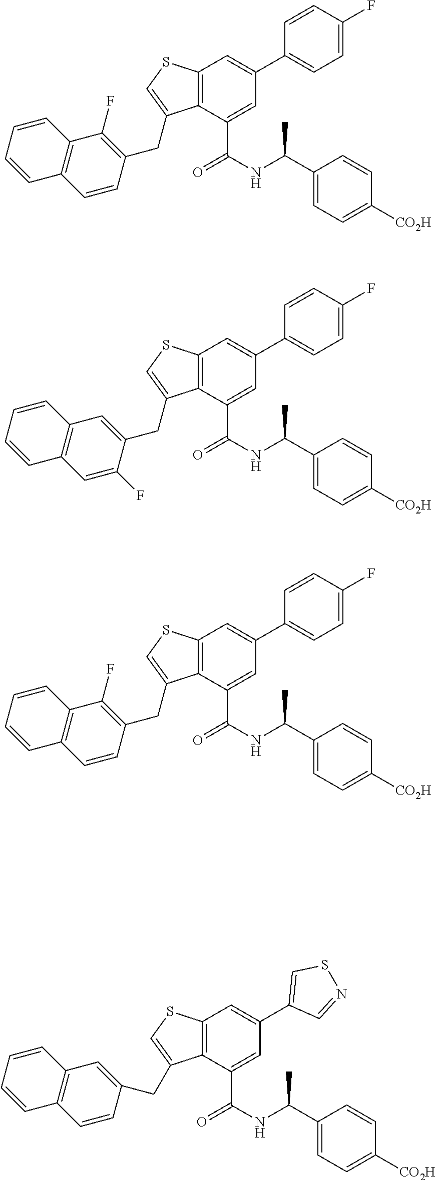 [SMILES: C[C@H](NC(=O)c1cc(-c2ccc(F)cc2)cc2scc(Cc3ccc4ccccc4c3F)c12)c1ccc(C(=O)O)cc1 ; C[C@H](NC(=O)c1cc(-c2ccc(F)cc2)cc2scc(Cc3cc4ccccc4cc3F)c12)c1ccc(C(=O)O)cc1 ; C[C@H](NC(=O)c1cc(-c2ccc(F)cc2)cc2scc(Cc3ccc4ccccc4c3F)c12)c1ccc(C(=O)O)cc1 ; C[C@H](NC(=O)c1cc(-c2cnsc2)cc2scc(Cc3ccc4ccccc4c3)c12)c1ccc(C(=O)O)cc1]

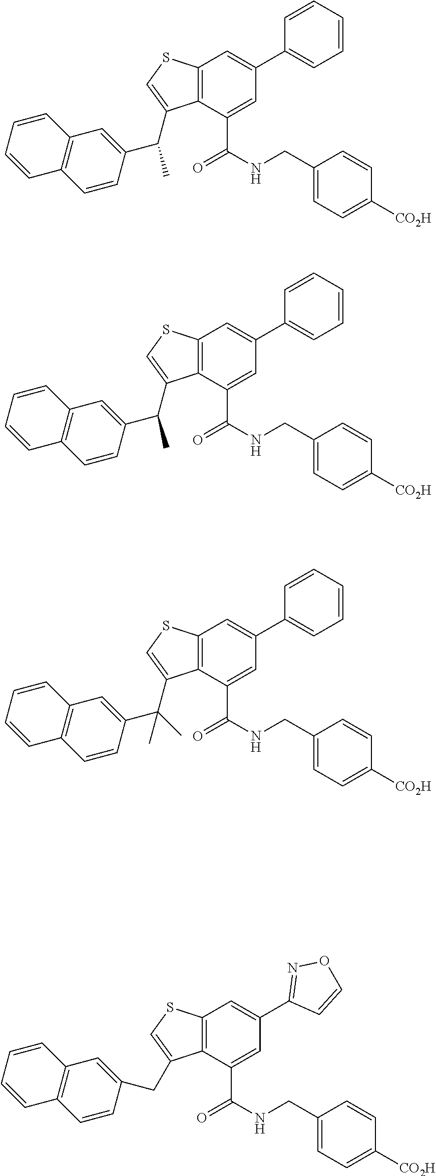 [SMILES: C[C@H](c1ccc2ccccc2c1)c1csc2cc(-c3ccccc3)cc(C(=O)NCc3ccc(C(=O)O)cc3)c12 ; C[C@@H](c1ccc2ccccc2c1)c1csc2cc(-c3ccccc3)cc(C(=O)NCc3ccc(C(=O)O)cc3)c12 ; CC(C)(c1ccc2ccccc2c1)c1csc2cc(-c3ccccc3)cc(C(=O)NCc3ccc(C(=O)O)cc3)c12 ; O=C(O)c1ccc(CNC(=O)c2cc(-c3ccon3)cc3scc(Cc4ccc5ccccc5c4)c23)cc1]

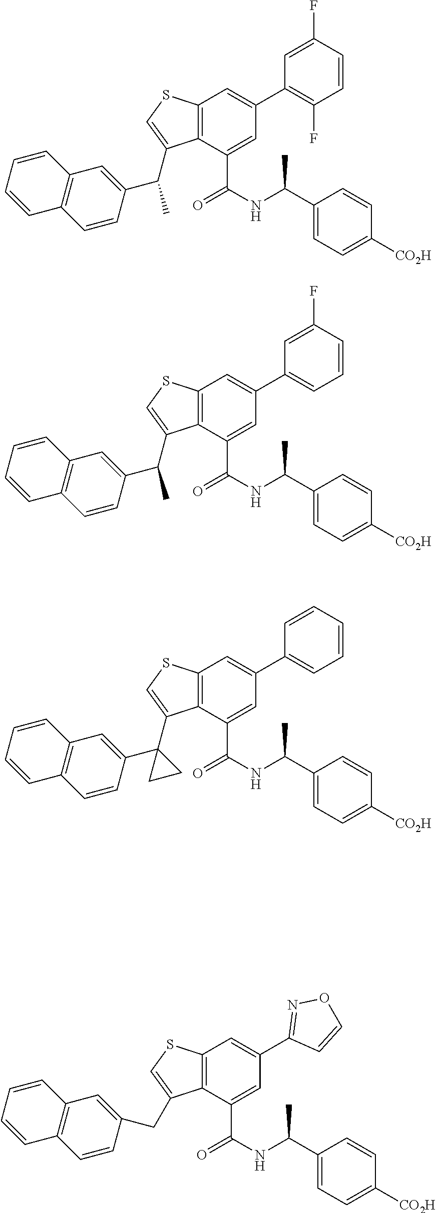

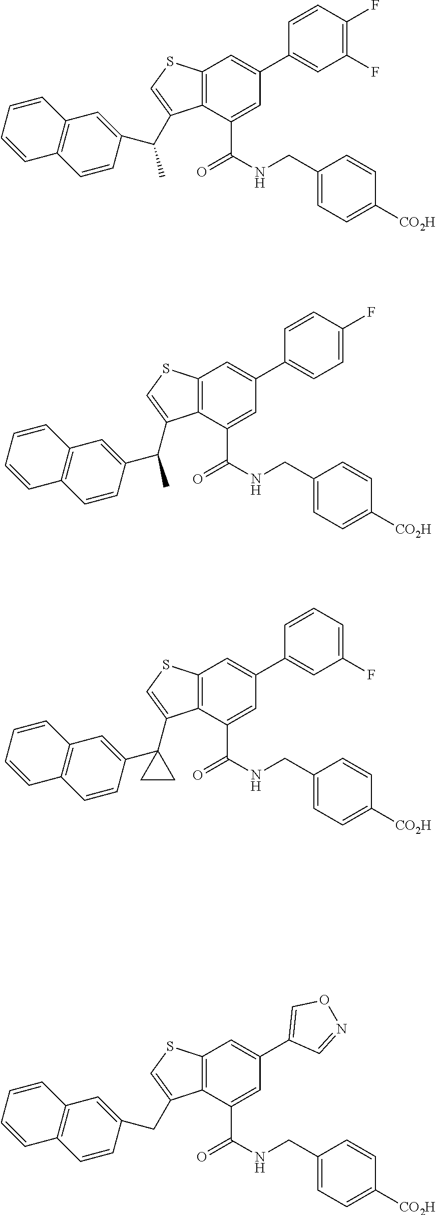 [SMILES: C[C@H](c1ccc2ccccc2c1)c1csc2cc(-c3ccc(F)c(F)c3)cc(C(=O)NCc3ccc(C(=O)O)cc3)c12 ; C[C@@H](c1ccc2ccccc2c1)c1csc2cc(-c3ccc(F)cc3)cc(C(=O)NCc3ccc(C(=O)O)cc3)c12 ; O=C(O)c1ccc(CNC(=O)c2cc(-c3cccc(F)c3)cc3scc(C4(c5ccc6ccccc6c5)CC4)c23)cc1 ; O=C(O)c1ccc(CNC(=O)c2cc(-c3cnoc3)cc3scc(Cc4ccc5ccccc5c4)c23)cc1]

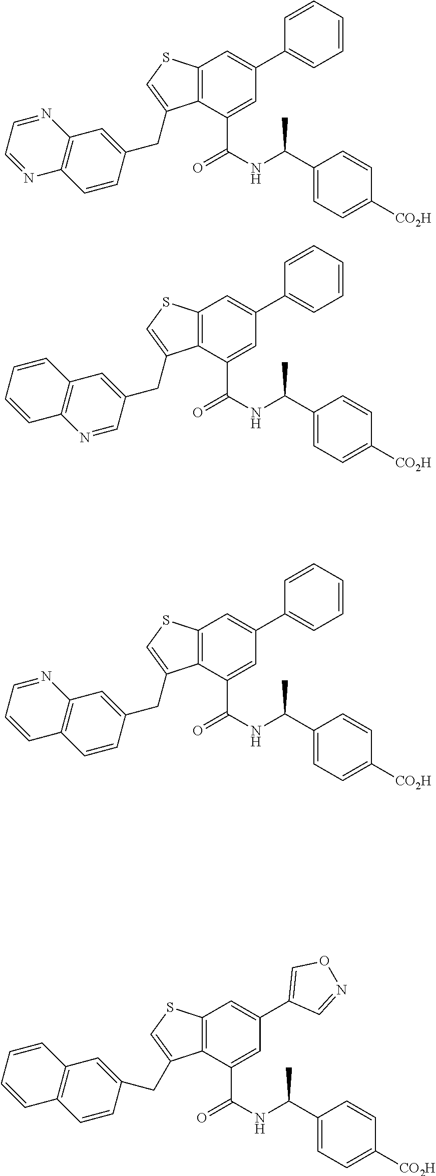

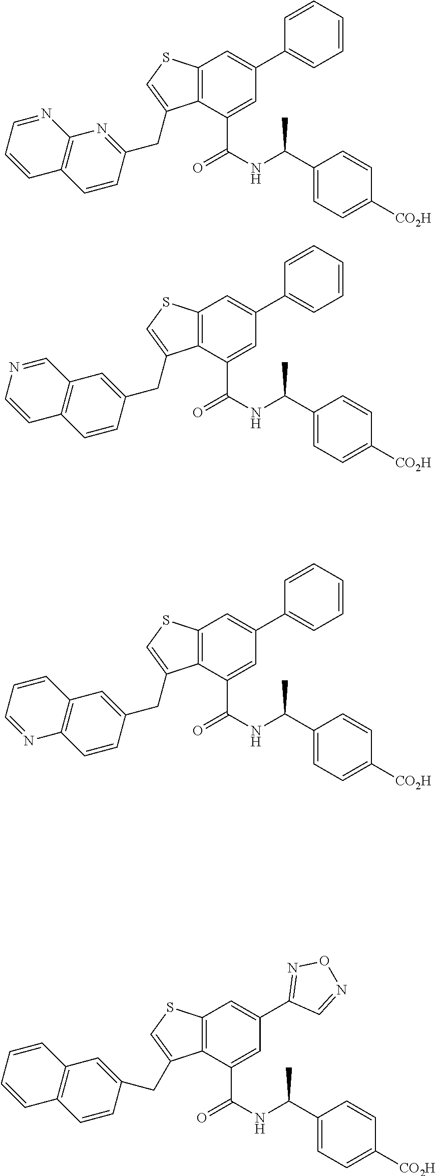

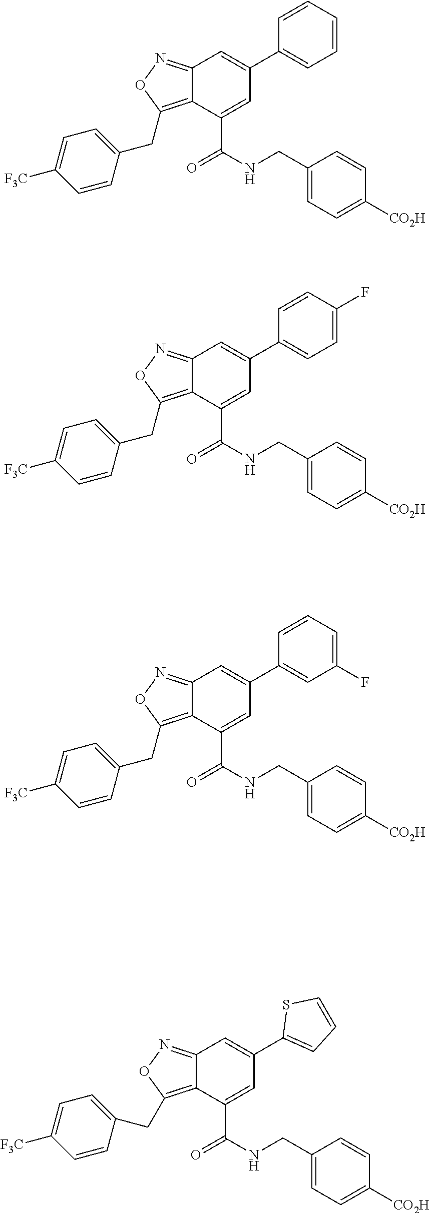 [SMILES: O=C(O)c1ccc(CNC(=O)c2cc(-c3ccccc3)cc3noc(Cc4ccc(C(F)(F)F)cc4)c23)cc1 ; O=C(O)c1ccc(CNC(=O)c2cc(-c3ccc(F)cc3)cc3noc(Cc4ccc(C(F)(F)F)cc4)c23)cc1 ; O=C(O)c1ccc(CNC(=O)c2cc(-c3cccc(F)c3)cc3noc(Cc4ccc(C(F)(F)F)cc4)c23)cc1 ; O=C(O)c1ccc(CNC(=O)c2cc(-c3cccs3)cc3noc(Cc4ccc(C(F)(F)F)cc4)c23)cc1]

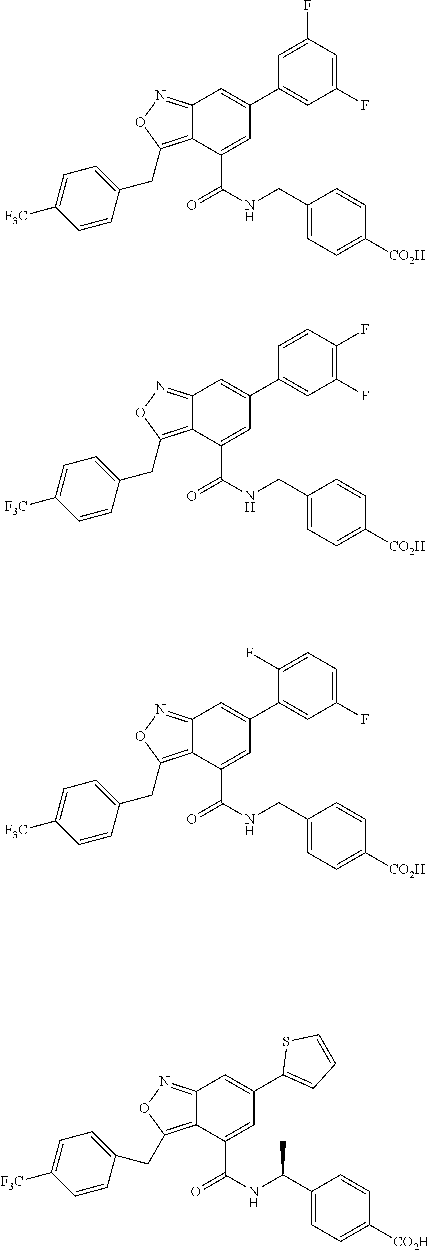 [SMILES: O=C(O)c1ccc(CNC(=O)c2cc(-c3cc(F)cc(F)c3)cc3noc(Cc4ccc(C(F)(F)F)cc4)c23)cc1 ; O=C(O)c1ccc(CNC(=O)c2cc(-c3ccc(F)c(F)c3)cc3noc(Cc4ccc(C(F)(F)F)cc4)c23)cc1 ; O=C(O)c1ccc(CNC(=O)c2cc(-c3cc(F)ccc3F)cc3noc(Cc4ccc(C(F)(F)F)cc4)c23)cc1 ; C[C@H](NC(=O)c1cc(-c2cccs2)cc2noc(Cc3ccc(C(F)(F)F)cc3)c12)c1ccc(C(=O)O)cc1]

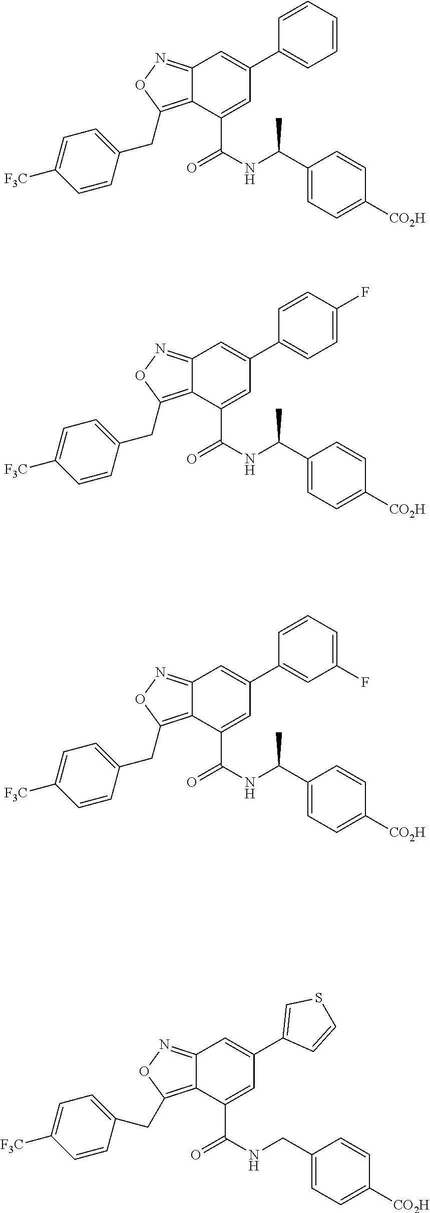 [SMILES: C[C@H](NC(=O)c1cc(-c2ccccc2)cc2noc(Cc3ccc(C(F)(F)F)cc3)c12)c1ccc(C(=O)O)cc1 ; C[C@H](NC(=O)c1cc(-c2ccc(F)cc2)cc2noc(Cc3ccc(C(F)(F)F)cc3)c12)c1ccc(C(=O)O)cc1 ; C[C@H](NC(=O)c1cc(-c2cccc(F)c2)cc2noc(Cc3ccc(C(F)(F)F)cc3)c12)c1ccc(C(=O)O)cc1 ; O=C(O)c1ccc(CNC(=O)c2cc(-c3ccsc3)cc3noc(Cc4ccc(C(F)(F)F)cc4)c23)cc1]

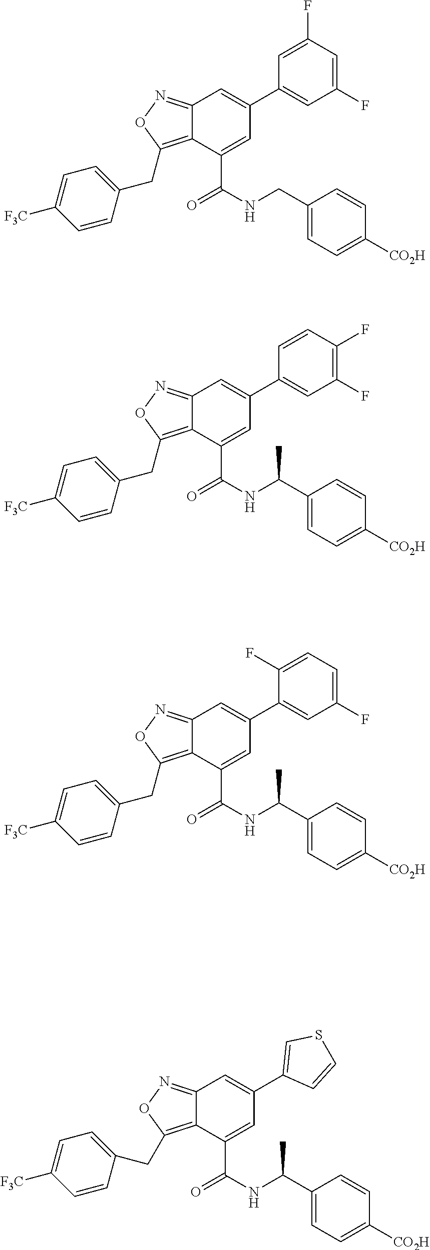 [SMILES: O=C(O)c1ccc(CNC(=O)c2cc(-c3cc(F)cc(F)c3)cc3noc(Cc4ccc(C(F)(F)F)cc4)c23)cc1 ; C[C@H](NC(=O)c1cc(-c2ccc(F)c(F)c2)cc2noc(Cc3ccc(C(F)(F)F)cc3)c12)c1ccc(C(=O)O)cc1 ; C[C@H](NC(=O)c1cc(-c2cc(F)ccc2F)cc2noc(Cc3ccc(C(F)(F)F)cc3)c12)c1ccc(C(=O)O)cc1 ; C[C@H](NC(=O)c1cc(-c2ccsc2)cc2noc(Cc3ccc(C(F)(F)F)cc3)c12)c1ccc(C(=O)O)cc1]

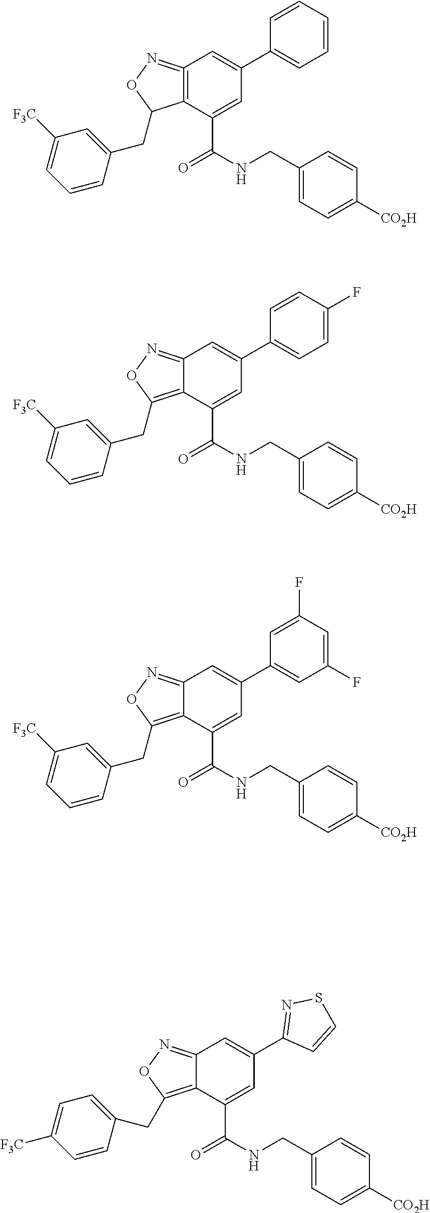

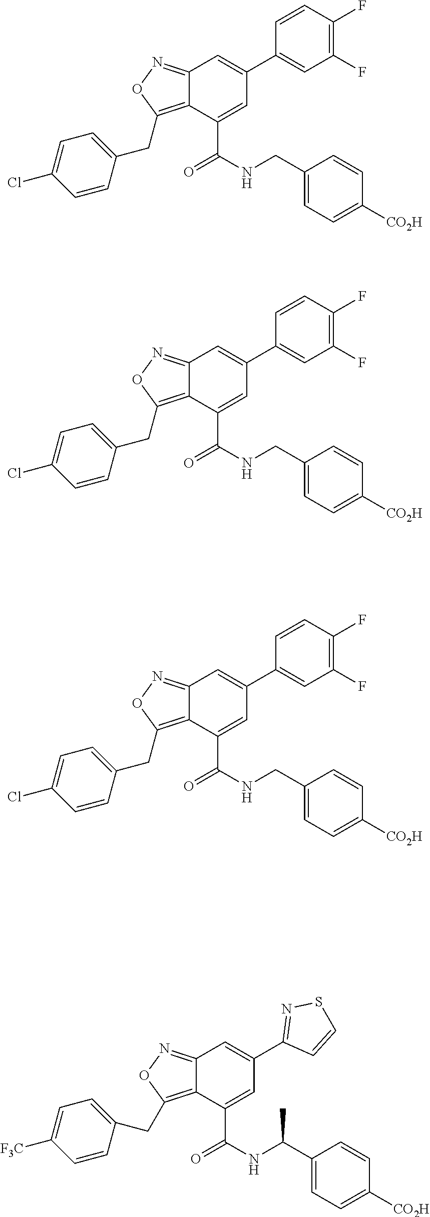 [SMILES: O=C(O)c1ccc(CNC(=O)c2cc(-c3ccc(F)c(F)c3)cc3noc(Cc4ccc(Cl)cc4)c23)cc1 ; O=C(O)c1ccc(CNC(=O)c2cc(-c3ccc(F)c(F)c3)cc3noc(Cc4ccc(Cl)cc4)c23)cc1 ; O=C(O)c1ccc(CNC(=O)c2cc(-c3ccc(F)c(F)c3)cc3noc(Cc4ccc(Cl)cc4)c23)cc1 ; C[C@H](NC(=O)c1cc(-c2ccsn2)cc2noc(Cc3ccc(C(F)(F)F)cc3)c12)c1ccc(C(=O)O)cc1]

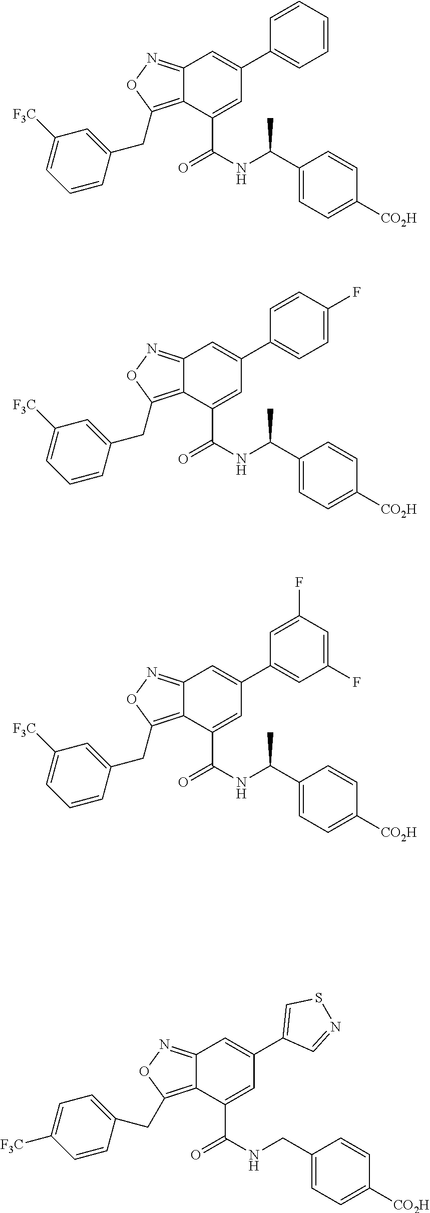 [SMILES: C[C@H](NC(=O)c1cc(-c2ccccc2)cc2noc(Cc3cccc(C(F)(F)F)c3)c12)c1ccc(C(=O)O)cc1 ; C[C@H](NC(=O)c1cc(-c2ccc(F)cc2)cc2noc(Cc3cccc(C(F)(F)F)c3)c12)c1ccc(C(=O)O)cc1 ; C[C@H](NC(=O)c1cc(-c2cc(F)cc(F)c2)cc2noc(Cc3cccc(C(F)(F)F)c3)c12)c1ccc(C(=O)O)cc1 ; O=C(O)c1ccc(CNC(=O)c2cc(-c3cnsc3)cc3noc(Cc4ccc(C(F)(F)F)cc4)c23)cc1]

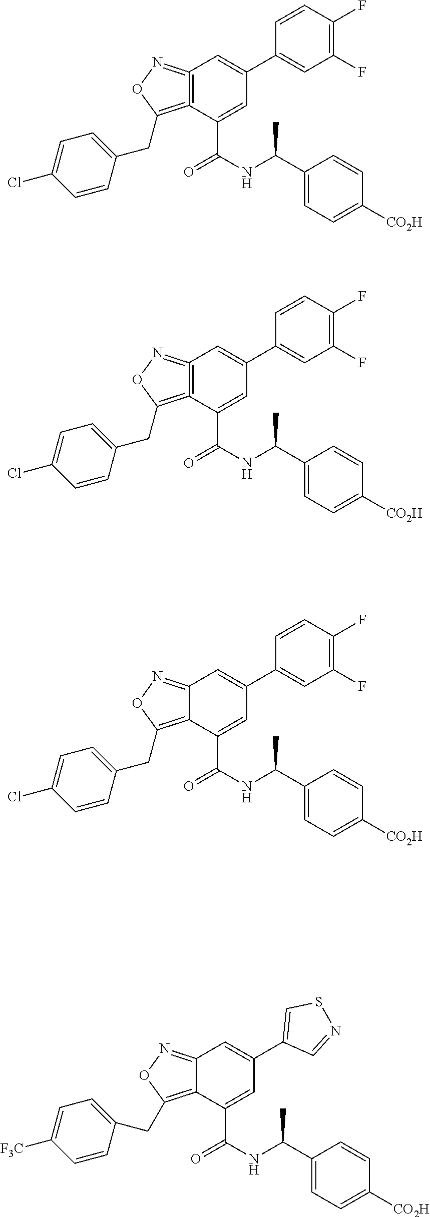

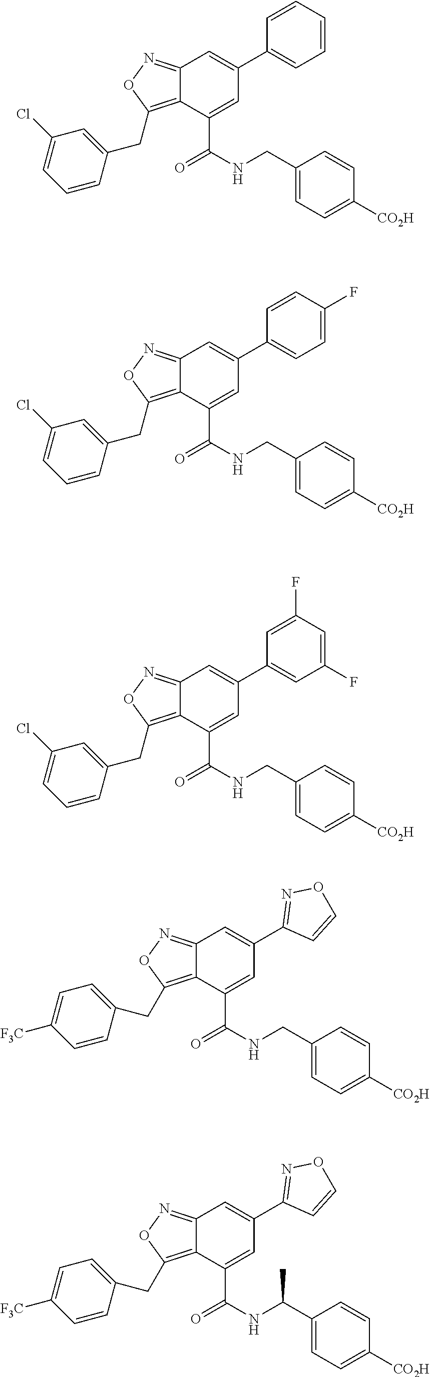 [SMILES: O=C(O)c1ccc(CNC(=O)c2cc(-c3ccccc3)cc3noc(Cc4cccc(Cl)c4)c23)cc1 ; O=C(O)c1ccc(CNC(=O)c2cc(-c3ccc(F)cc3)cc3noc(Cc4cccc(Cl)c4)c23)cc1 ; O=C(O)c1ccc(CNC(=O)c2cc(-c3cc(F)cc(F)c3)cc3noc(Cc4cccc(Cl)c4)c23)cc1 ; O=C(O)c1ccc(CNC(=O)c2cc(-c3ccon3)cc3noc(Cc4ccc(C(F)(F)F)cc4)c23)cc1 ; C[C@H](NC(=O)c1cc(-c2ccon2)cc2noc(Cc3ccc(C(F)(F)F)cc3)c12)c1ccc(C(=O)O)cc1]

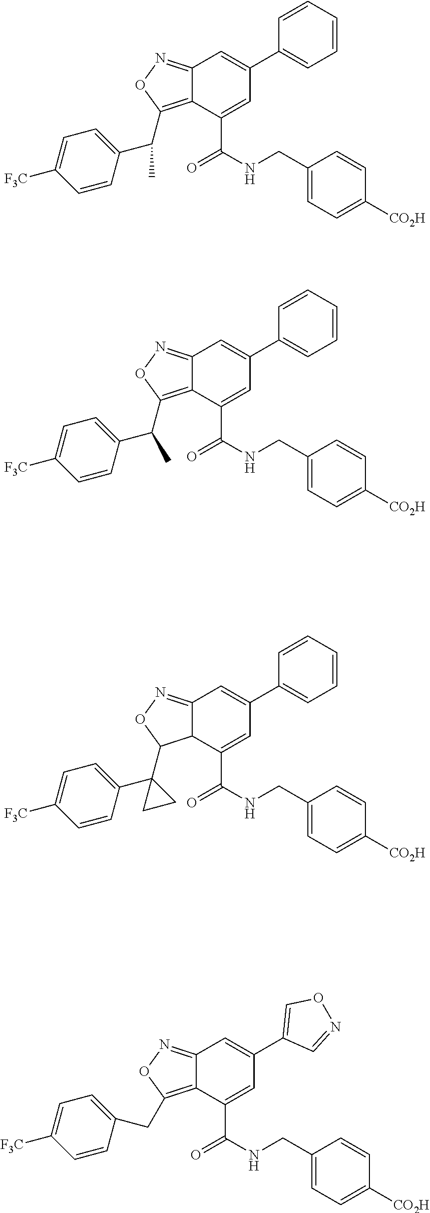 [SMILES: C[C@H](c1ccc(C(F)(F)F)cc1)c1onc2cc(-c3ccccc3)cc(C(=O)NCc3ccc(C(=O)O)cc3)c12 ; C[C@@H](c1ccc(C(F)(F)F)cc1)c1onc2cc(-c3ccccc3)cc(C(=O)NCc3ccc(C(=O)O)cc3)c12 ; O=C(NCc1ccc(C(=O)O)cc1)C1=CC(c2ccccc2)=CC2=NOC(C3(c4ccc(C(F)(F)F)cc4)CC3)C12 ; O=C(O)c1ccc(CNC(=O)c2cc(-c3cnoc3)cc3noc(Cc4ccc(C(F)(F)F)cc4)c23)cc1]

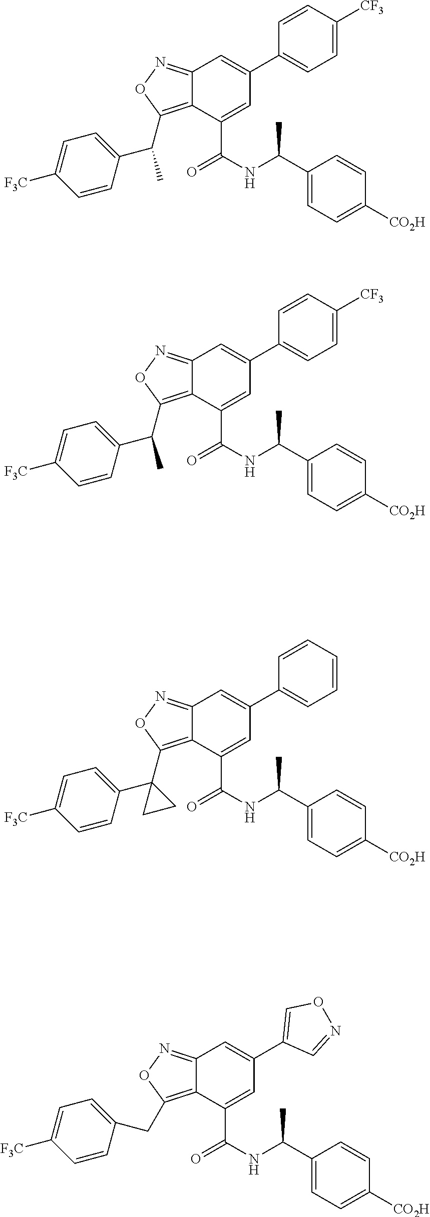 [SMILES: C[C@H](c1ccc(C(F)(F)F)cc1)c1onc2cc(-c3ccc(C(F)(F)F)cc3)cc(C(=O)N[C@@H](C)c3ccc(C(=O)O)cc3)c12 ; C[C@@H](c1ccc(C(F)(F)F)cc1)c1onc2cc(-c3ccc(C(F)(F)F)cc3)cc(C(=O)N[C@@H](C)c3ccc(C(=O)O)cc3)c12 ; C[C@H](NC(=O)c1cc(-c2ccccc2)cc2noc(C3(c4ccc(C(F)(F)F)cc4)CC3)c12)c1ccc(C(=O)O)cc1 ; C[C@H](NC(=O)c1cc(-c2cnoc2)cc2noc(Cc3ccc(C(F)(F)F)cc3)c12)c1ccc(C(=O)O)cc1]

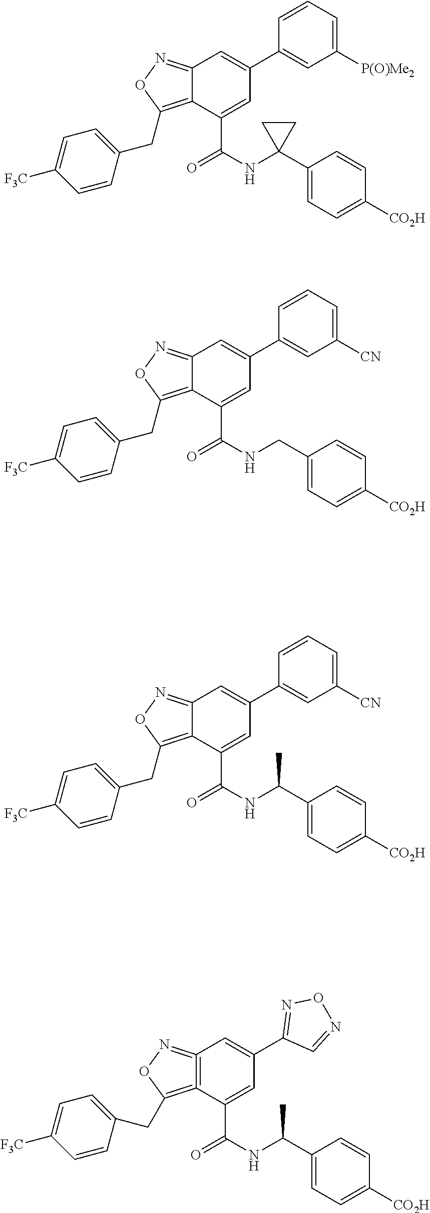

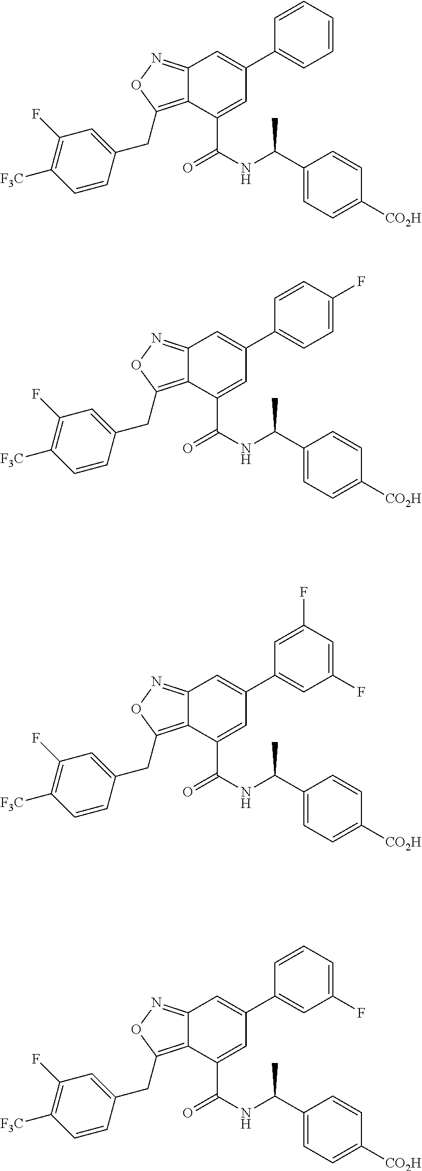 [SMILES: C[C@H](NC(=O)c1cc(-c2ccccc2)cc2noc(Cc3ccc(C(F)(F)F)c(F)c3)c12)c1ccc(C(=O)O)cc1 ; C[C@H](NC(=O)c1cc(-c2ccc(F)cc2)cc2noc(Cc3ccc(C(F)(F)F)c(F)c3)c12)c1ccc(C(=O)O)cc1 ; C[C@H](NC(=O)c1cc(-c2cc(F)cc(F)c2)cc2noc(Cc3ccc(C(F)(F)F)c(F)c3)c12)c1ccc(C(=O)O)cc1 ; C[C@H](NC(=O)c1cc(-c2cccc(F)c2)cc2noc(Cc3ccc(C(F)(F)F)c(F)c3)c12)c1ccc(C(=O)O)cc1]

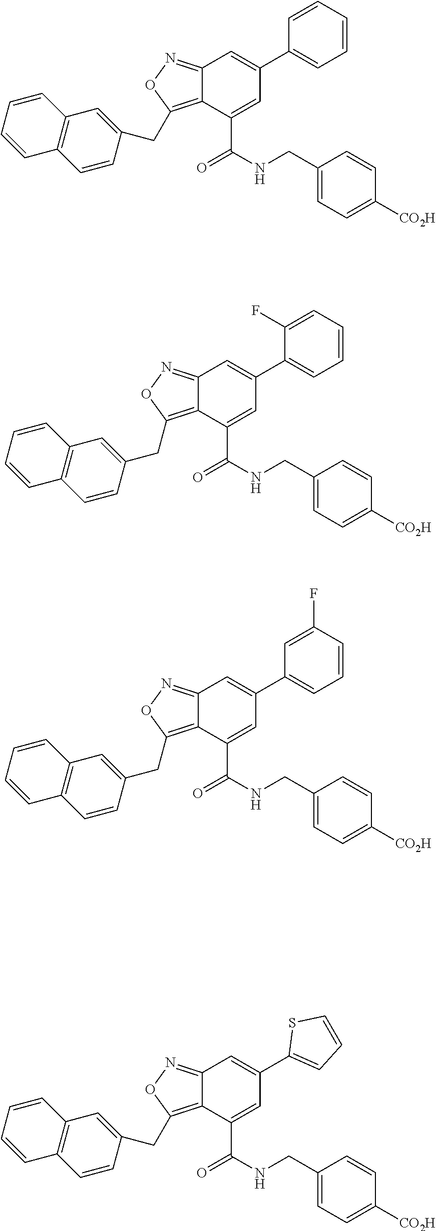 [SMILES: O=C(O)c1ccc(CNC(=O)c2cc(-c3ccccc3)cc3noc(Cc4ccc5ccccc5c4)c23)cc1 ; O=C(O)c1ccc(CNC(=O)c2cc(-c3ccccc3F)cc3noc(Cc4ccc5ccccc5c4)c23)cc1 ; O=C(O)c1ccc(CNC(=O)c2cc(-c3cccc(F)c3)cc3noc(Cc4ccc5ccccc5c4)c23)cc1 ; O=C(O)c1ccc(CNC(=O)c2cc(-c3cccs3)cc3noc(Cc4ccc5ccccc5c4)c23)cc1]

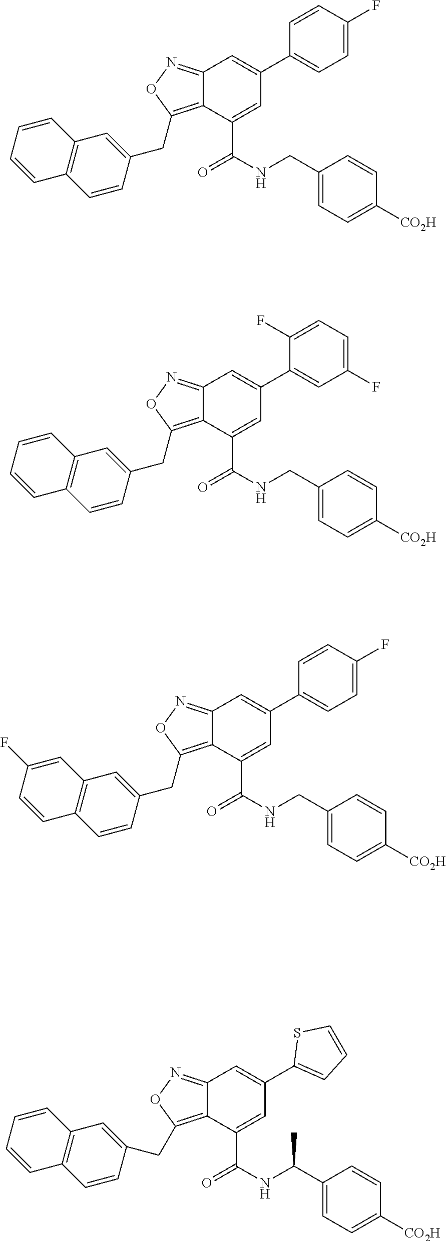 [SMILES: O=C(O)c1ccc(CNC(=O)c2cc(-c3ccc(F)cc3)cc3noc(Cc4ccc5ccccc5c4)c23)cc1 ; O=C(O)c1ccc(CNC(=O)c2cc(-c3cc(F)ccc3F)cc3noc(Cc4ccc5ccccc5c4)c23)cc1 ; O=C(O)c1ccc(CNC(=O)c2cc(-c3ccc(F)cc3)cc3noc(Cc4ccc5ccc(F)cc5c4)c23)cc1 ; C[C@H](NC(=O)c1cc(-c2cccs2)cc2noc(Cc3ccc4ccccc4c3)c12)c1ccc(C(=O)O)cc1]

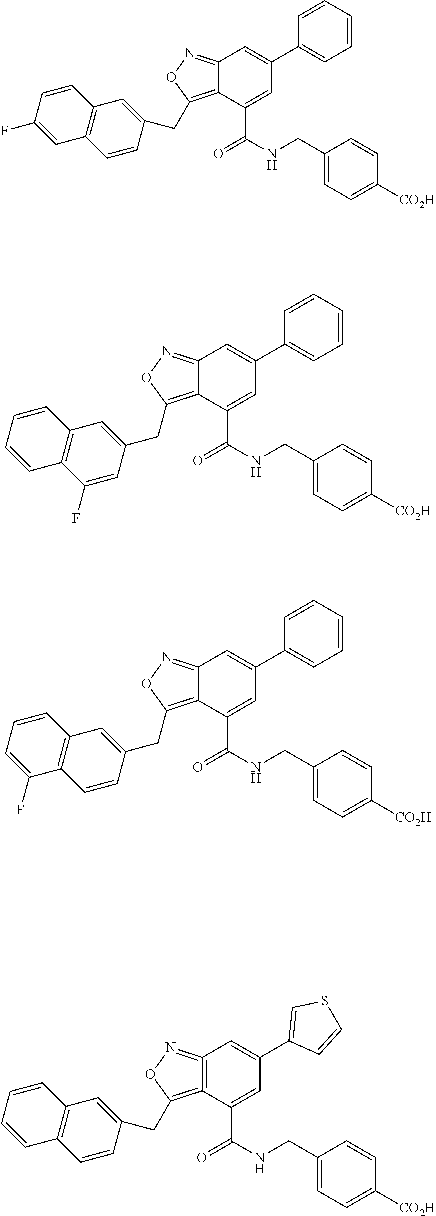

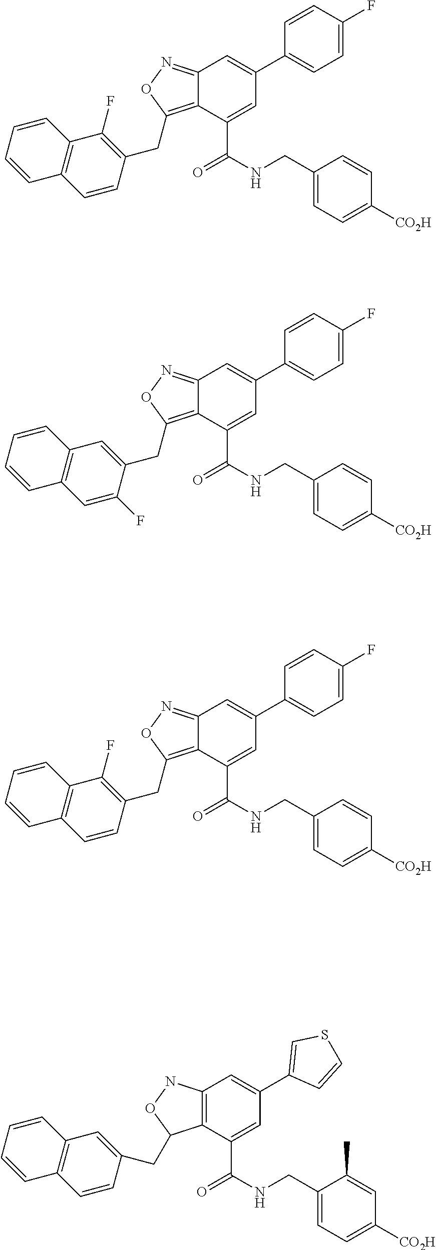

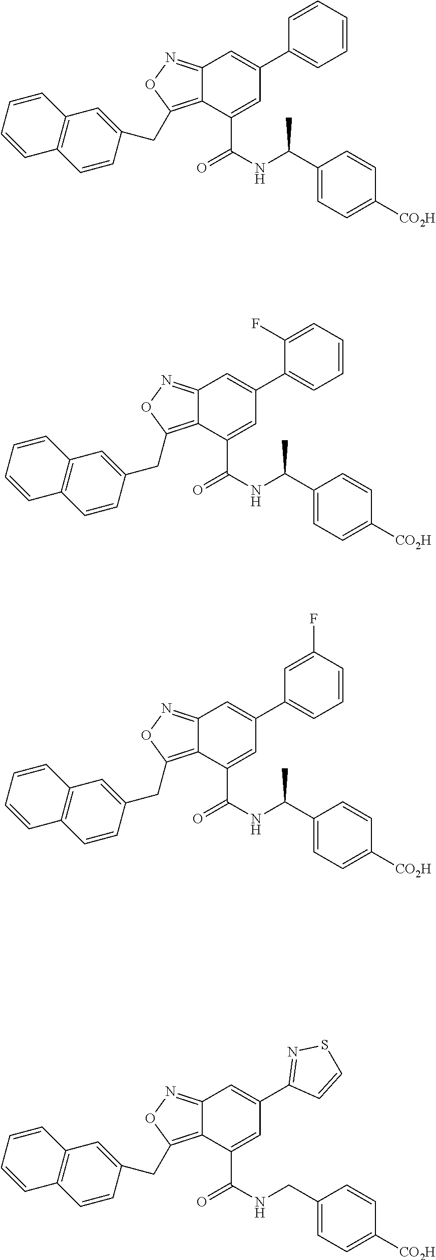 [SMILES: C[C@H](NC(=O)c1cc(-c2ccccc2)cc2noc(Cc3ccc4ccccc4c3)c12)c1ccc(C(=O)O)cc1 ; C[C@H](NC(=O)c1cc(-c2ccccc2F)cc2noc(Cc3ccc4ccccc4c3)c12)c1ccc(C(=O)O)cc1 ; C[C@H](NC(=O)c1cc(-c2cccc(F)c2)cc2noc(Cc3ccc4ccccc4c3)c12)c1ccc(C(=O)O)cc1 ; O=C(O)c1ccc(CNC(=O)c2cc(-c3ccsn3)cc3noc(Cc4ccc5ccccc5c4)c23)cc1]

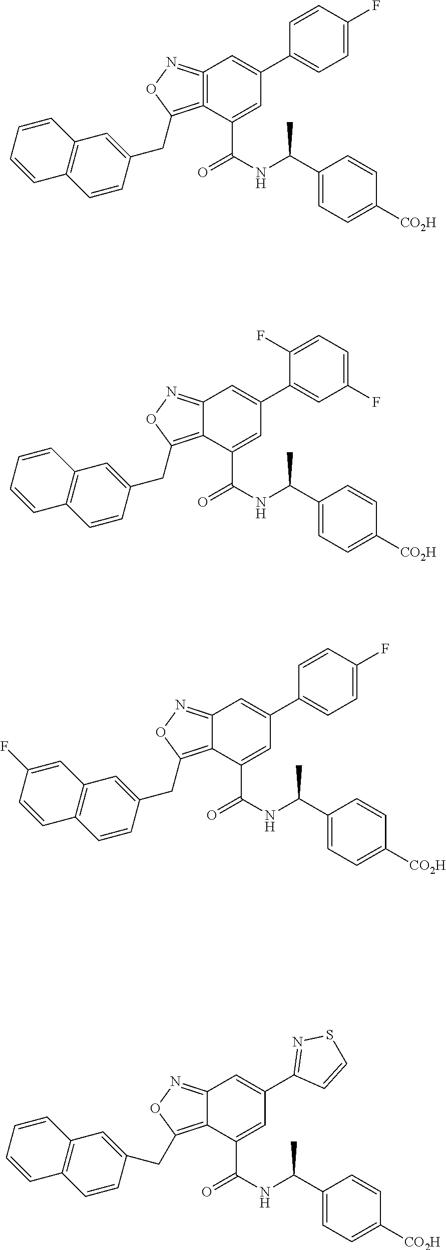

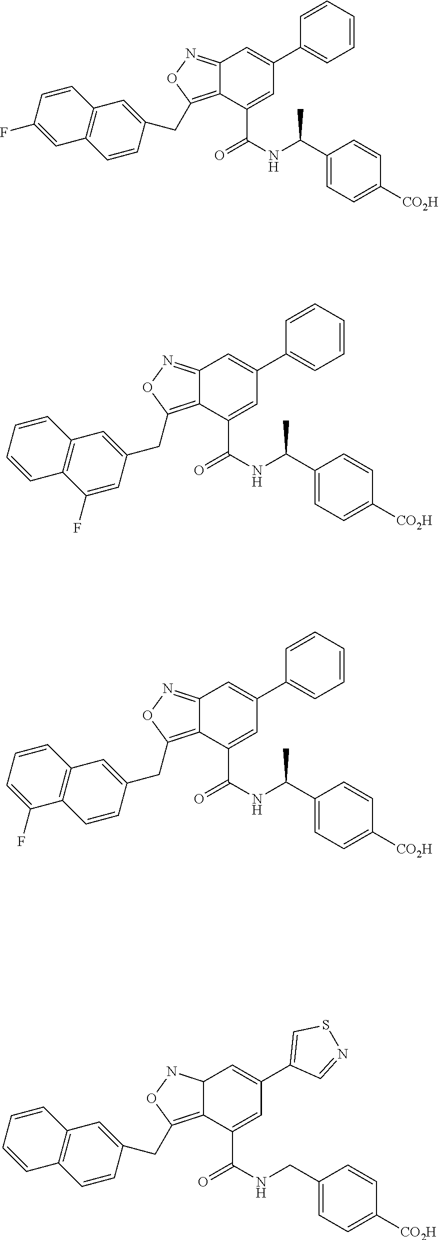 [SMILES: C[C@H](NC(=O)c1cc(-c2ccccc2)cc2noc(Cc3ccc4cc(F)ccc4c3)c12)c1ccc(C(=O)O)cc1 ; C[C@H](NC(=O)c1cc(-c2ccccc2)cc2noc(Cc3cc(F)c4ccccc4c3)c12)c1ccc(C(=O)O)cc1 ; C[C@H](NC(=O)c1cc(-c2ccccc2)cc2noc(Cc3ccc4c(F)cccc4c3)c12)c1ccc(C(=O)O)cc1 ; O=C(NCc1ccc(C(=O)O)cc1)C1=CC(c2cnsc2)=CC2NOC(Cc3ccc4ccccc4c3)=C12]

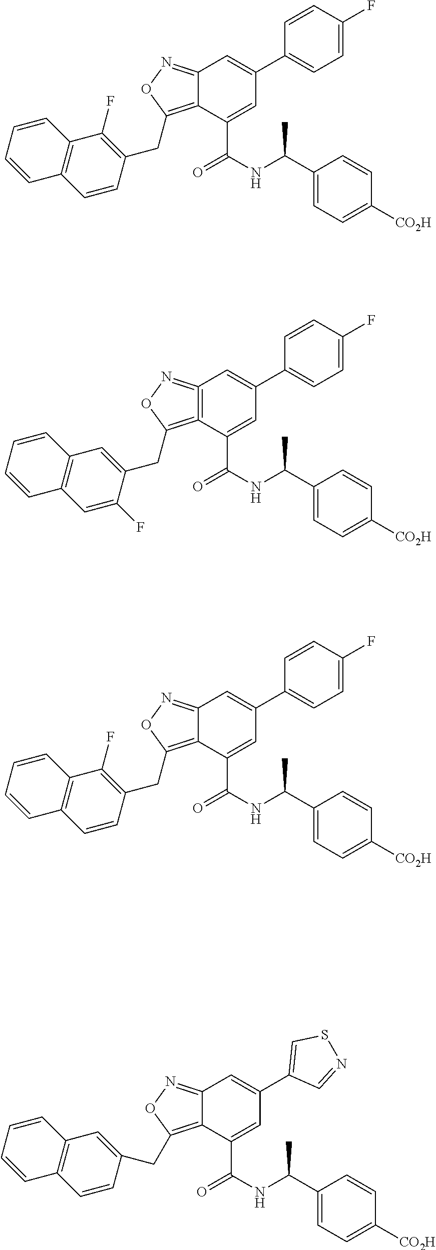

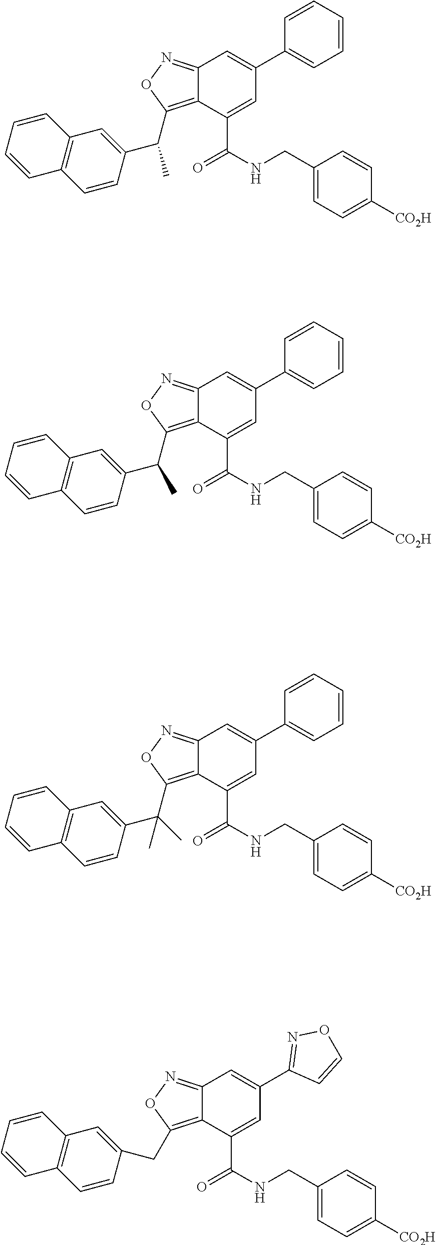 [SMILES: C[C@H](c1ccc2ccccc2c1)c1onc2cc(-c3ccccc3)cc(C(=O)NCc3ccc(C(=O)O)cc3)c12 ; C[C@@H](c1ccc2ccccc2c1)c1onc2cc(-c3ccccc3)cc(C(=O)NCc3ccc(C(=O)O)cc3)c12 ; CC(C)(c1ccc2ccccc2c1)c1onc2cc(-c3ccccc3)cc(C(=O)NCc3ccc(C(=O)O)cc3)c12 ; O=C(O)c1ccc(CNC(=O)c2cc(-c3ccon3)cc3noc(Cc4ccc5ccccc5c4)c23)cc1]

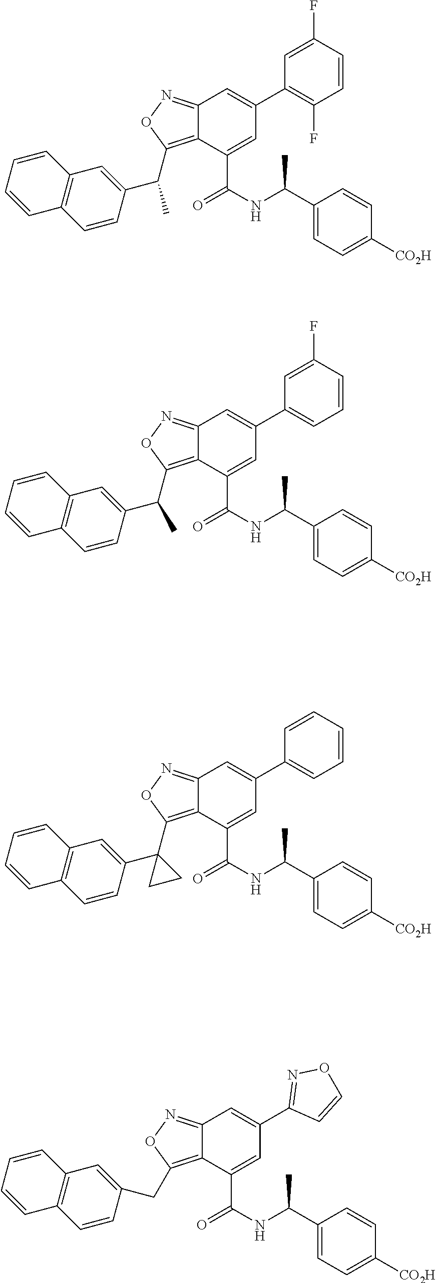 [SMILES: C[C@H](c1ccc2ccccc2c1)c1onc2cc(-c3cc(F)ccc3F)cc(C(=O)N[C@@H](C)c3ccc(C(=O)O)cc3)c12 ; C[C@@H](c1ccc2ccccc2c1)c1onc2cc(-c3cccc(F)c3)cc(C(=O)N[C@@H](C)c3ccc(C(=O)O)cc3)c12 ; C[C@H](NC(=O)c1cc(-c2ccccc2)cc2noc(C3(c4ccc5ccccc5c4)CC3)c12)c1ccc(C(=O)O)cc1 ; C[C@H](NC(=O)c1cc(-c2ccon2)cc2noc(Cc3ccc4ccccc4c3)c12)c1ccc(C(=O)O)cc1]

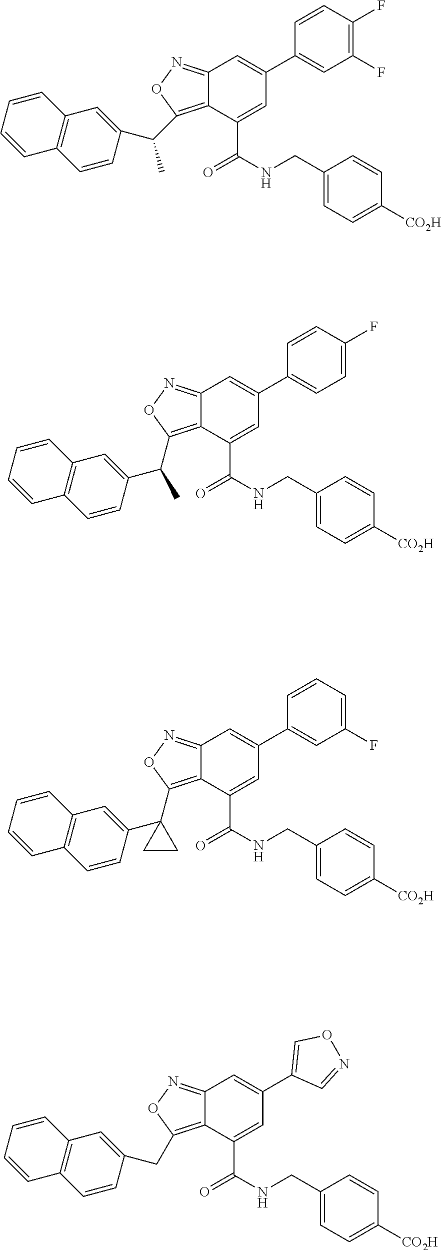 [SMILES: C[C@H](c1ccc2ccccc2c1)c1onc2cc(-c3ccc(F)c(F)c3)cc(C(=O)NCc3ccc(C(=O)O)cc3)c12 ; C[C@@H](c1ccc2ccccc2c1)c1onc2cc(-c3ccc(F)cc3)cc(C(=O)NCc3ccc(C(=O)O)cc3)c12 ; O=C(O)c1ccc(CNC(=O)c2cc(-c3cccc(F)c3)cc3noc(C4(c5ccc6ccccc6c5)CC4)c23)cc1 ; O=C(O)c1ccc(CNC(=O)c2cc(-c3cnoc3)cc3noc(Cc4ccc5ccccc5c4)c23)cc1]

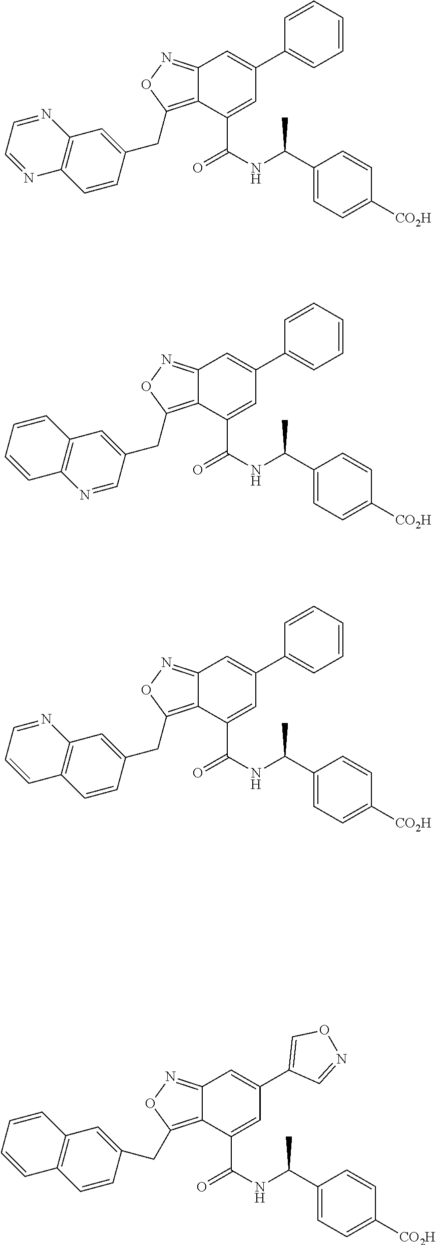 [SMILES: C[C@H](NC(=O)c1cc(-c2ccccc2)cc2noc(Cc3ccc4nccnc4c3)c12)c1ccc(C(=O)O)cc1 ; C[C@H](NC(=O)c1cc(-c2ccccc2)cc2noc(Cc3cnc4ccccc4c3)c12)c1ccc(C(=O)O)cc1 ; C[C@H](NC(=O)c1cc(-c2ccccc2)cc2noc(Cc3ccc4cccnc4c3)c12)c1ccc(C(=O)O)cc1 ; C[C@H](NC(=O)c1cc(-c2cnoc2)cc2noc(Cc3ccc4ccccc4c3)c12)c1ccc(C(=O)O)cc1]

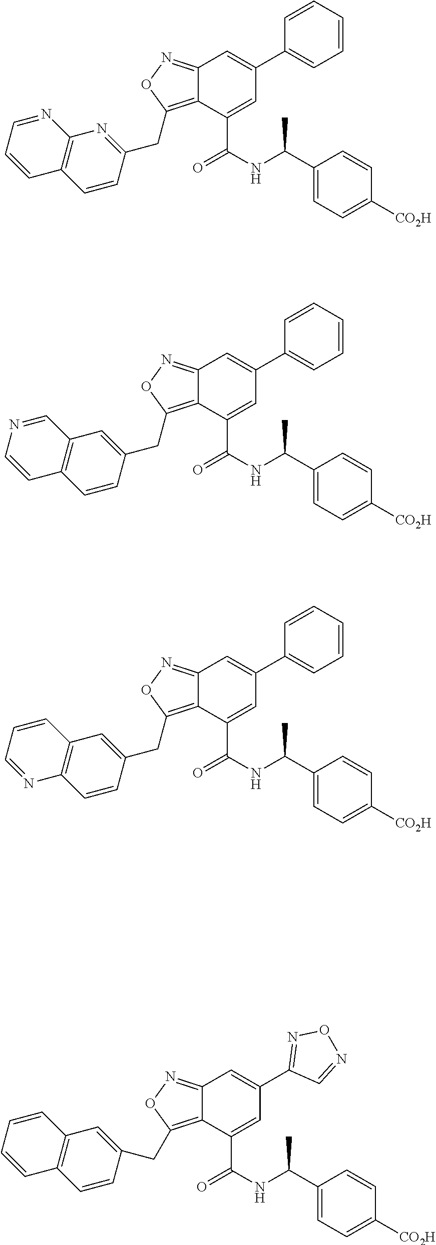 [SMILES: C[C@H](NC(=O)c1cc(-c2ccccc2)cc2noc(Cc3ccc4cccnc4n3)c12)c1ccc(C(=O)O)cc1 ; C[C@H](NC(=O)c1cc(-c2ccccc2)cc2noc(Cc3ccc4ccncc4c3)c12)c1ccc(C(=O)O)cc1 ; C[C@H](NC(=O)c1cc(-c2ccccc2)cc2noc(Cc3ccc4ncccc4c3)c12)c1ccc(C(=O)O)cc1 ; C[C@H](NC(=O)c1cc(-c2cnon2)cc2noc(Cc3ccc4ccccc4c3)c12)c1ccc(C(=O)O)cc1]

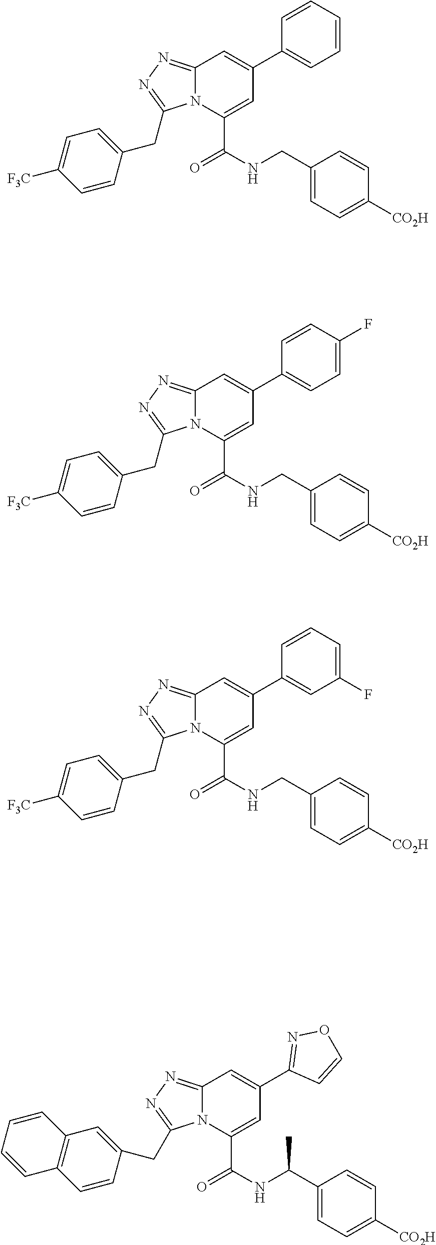 [SMILES: O=C(O)c1ccc(CNC(=O)c2cc(-c3ccccc3)cc3nnc(Cc4ccc(C(F)(F)F)cc4)n23)cc1 ; O=C(O)c1ccc(CNC(=O)c2cc(-c3ccc(F)cc3)cc3nnc(Cc4ccc(C(F)(F)F)cc4)n23)cc1 ; O=C(O)c1ccc(CNC(=O)c2cc(-c3cccc(F)c3)cc3nnc(Cc4ccc(C(F)(F)F)cc4)n23)cc1 ; C[C@H](NC(=O)c1cc(-c2ccon2)cc2nnc(Cc3ccc4ccccc4c3)n12)c1ccc(C(=O)O)cc1]

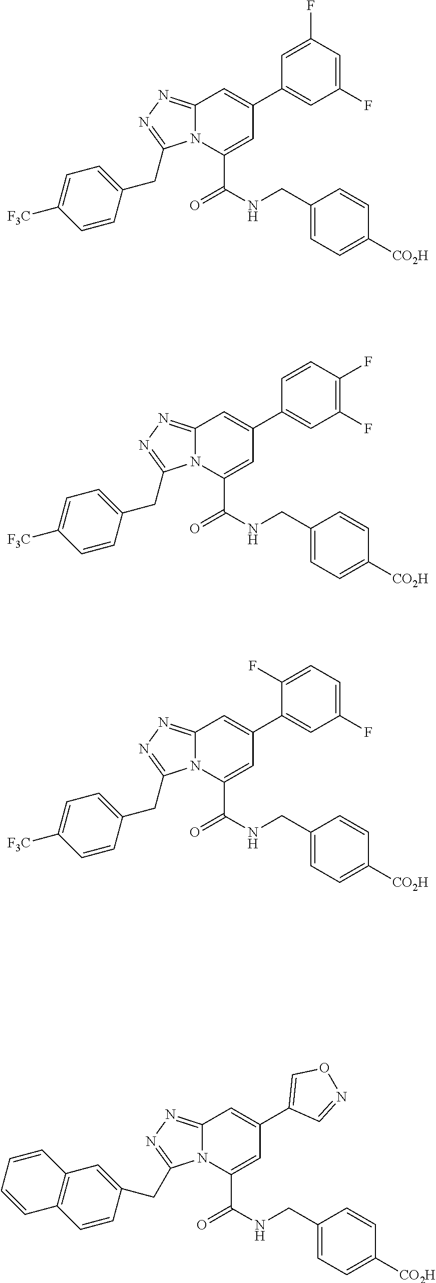 [SMILES: O=C(O)c1ccc(CNC(=O)c2cc(-c3cc(F)cc(F)c3)cc3nnc(Cc4ccc(C(F)(F)F)cc4)n23)cc1 ; O=C(O)c1ccc(CNC(=O)c2cc(-c3ccc(F)c(F)c3)cc3nnc(Cc4ccc(C(F)(F)F)cc4)n23)cc1 ; O=C(O)c1ccc(CNC(=O)c2cc(-c3cc(F)ccc3F)cc3nnc(Cc4ccc(C(F)(F)F)cc4)n23)cc1 ; O=C(O)c1ccc(CNC(=O)c2cc(-c3cnoc3)cc3nnc(Cc4ccc5ccccc5c4)n23)cc1]

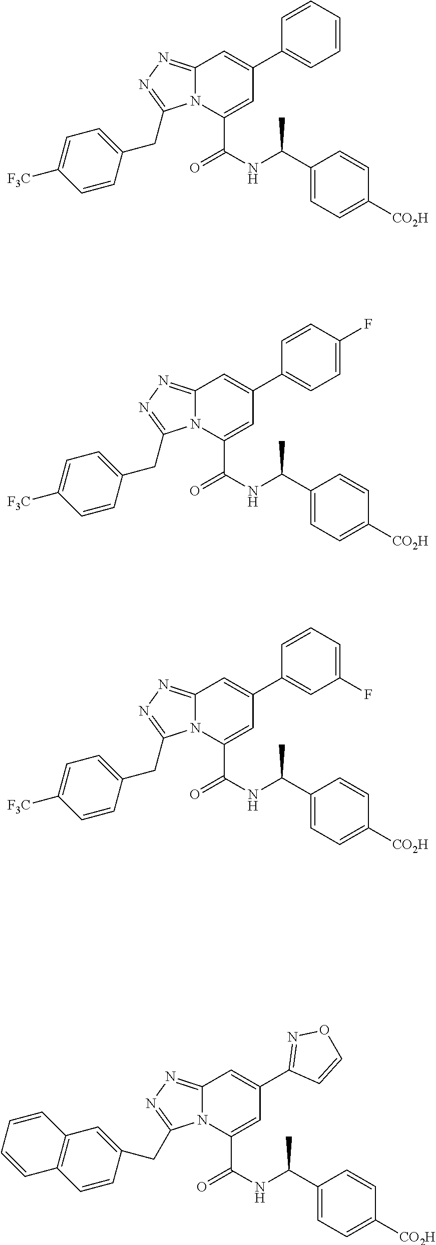 [SMILES: C[C@H](NC(=O)c1cc(-c2ccccc2)cc2nnc(Cc3ccc(C(F)(F)F)cc3)n12)c1ccc(C(=O)O)cc1 ; C[C@H](NC(=O)c1cc(-c2ccc(F)cc2)cc2nnc(Cc3ccc(C(F)(F)F)cc3)n12)c1ccc(C(=O)O)cc1 ; C[C@H](NC(=O)c1cc(-c2cccc(F)c2)cc2nnc(Cc3ccc(C(F)(F)F)cc3)n12)c1ccc(C(=O)O)cc1 ; C[C@H](NC(=O)c1cc(-c2ccon2)cc2nnc(Cc3ccc4ccccc4c3)n12)c1ccc(C(=O)O)cc1]

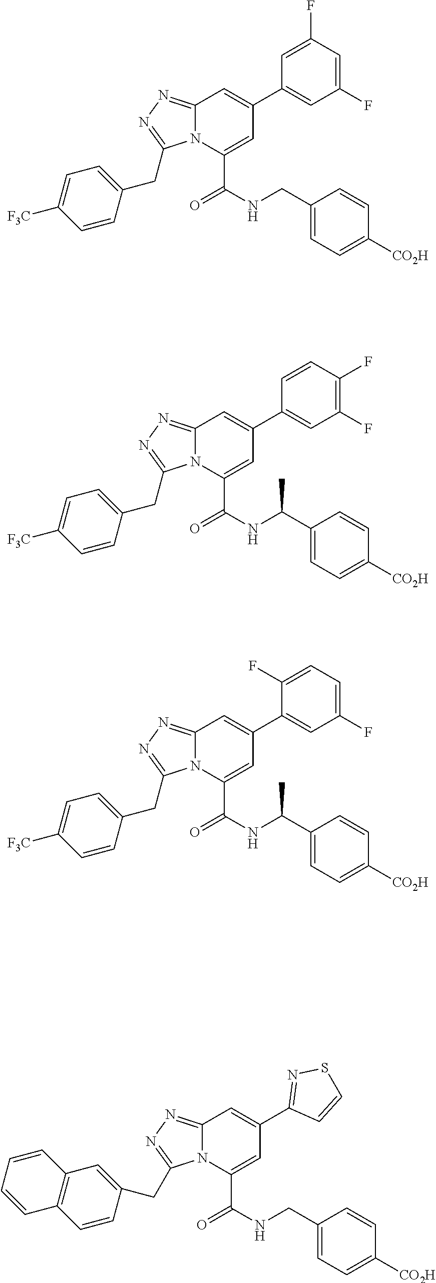 [SMILES: O=C(O)c1ccc(CNC(=O)c2cc(-c3cc(F)cc(F)c3)cc3nnc(Cc4ccc(C(F)(F)F)cc4)n23)cc1 ; C[C@H](NC(=O)c1cc(-c2ccc(F)c(F)c2)cc2nnc(Cc3ccc(C(F)(F)F)cc3)n12)c1ccc(C(=O)O)cc1 ; C[C@H](NC(=O)c1cc(-c2cc(F)ccc2F)cc2nnc(Cc3ccc(C(F)(F)F)cc3)n12)c1ccc(C(=O)O)cc1 ; O=C(O)c1ccc(CNC(=O)c2cc(-c3ccsn3)cc3nnc(Cc4ccc5ccccc5c4)n23)cc1]

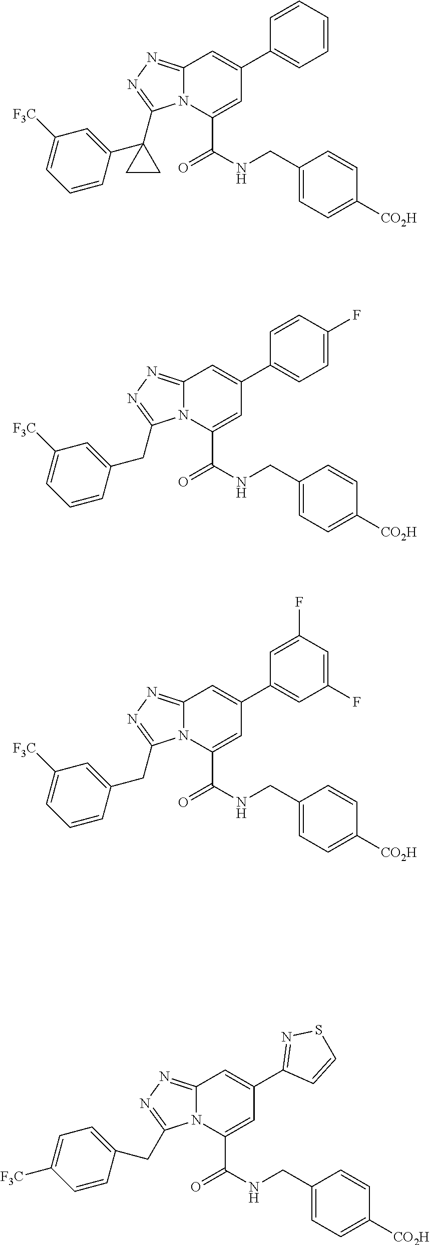 [SMILES: O=C(O)c1ccc(CNC(=O)c2cc(-c3ccccc3)cc3nnc(C4(c5cccc(C(F)(F)F)c5)CC4)n23)cc1 ; O=C(O)c1ccc(CNC(=O)c2cc(-c3ccc(F)cc3)cc3nnc(Cc4cccc(C(F)(F)F)c4)n23)cc1 ; O=C(O)c1ccc(CNC(=O)c2cc(-c3cc(F)cc(F)c3)cc3nnc(Cc4cccc(C(F)(F)F)c4)n23)cc1 ; O=C(O)c1ccc(CNC(=O)c2cc(-c3ccsn3)cc3nnc(Cc4ccc(C(F)(F)F)cc4)n23)cc1]

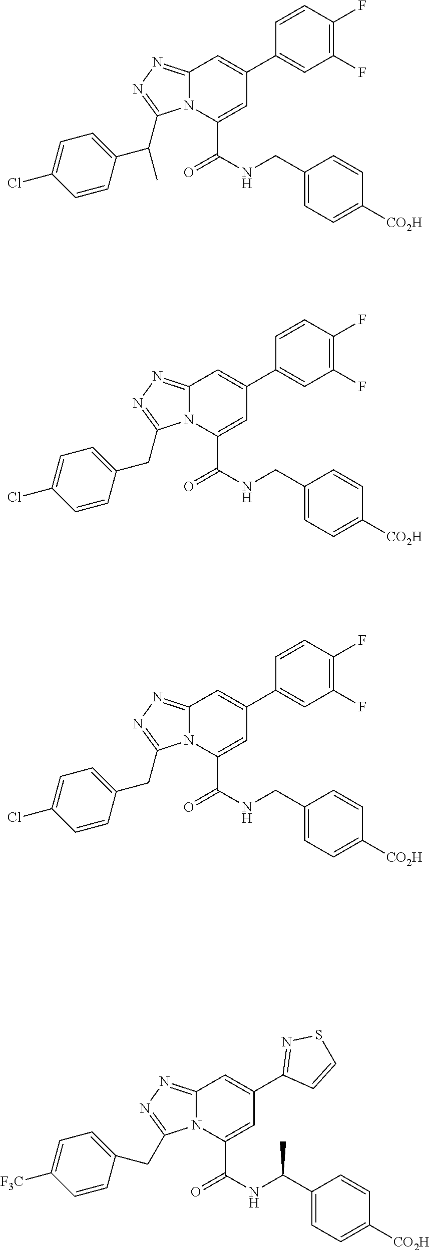 [SMILES: CC(c1ccc(Cl)cc1)c1nnc2cc(-c3ccc(F)c(F)c3)cc(C(=O)NCc3ccc(C(=O)O)cc3)n12 ; O=C(O)c1ccc(CNC(=O)c2cc(-c3ccc(F)c(F)c3)cc3nnc(Cc4ccc(Cl)cc4)n23)cc1 ; O=C(O)c1ccc(CNC(=O)c2cc(-c3ccc(F)c(F)c3)cc3nnc(Cc4ccc(Cl)cc4)n23)cc1 ; C[C@H](NC(=O)c1cc(-c2ccsn2)cc2nnc(Cc3ccc(C(F)(F)F)cc3)n12)c1ccc(C(=O)O)cc1]

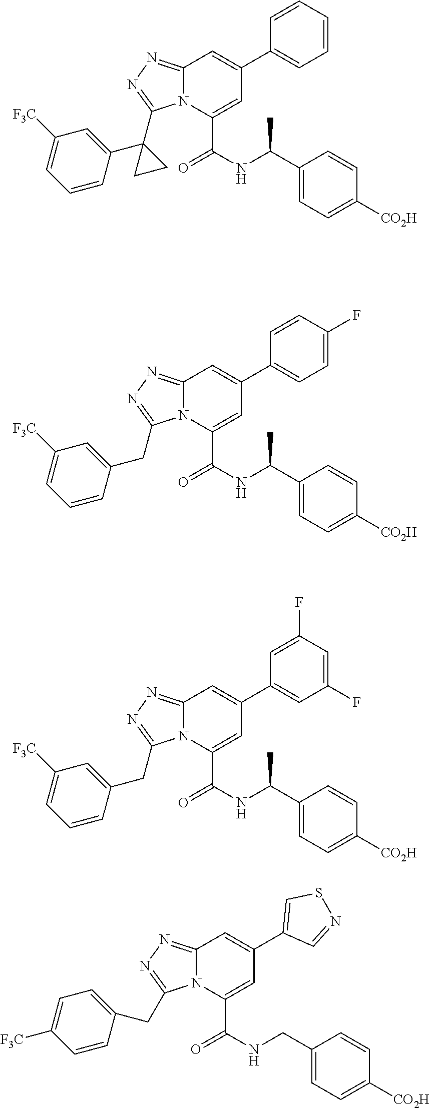

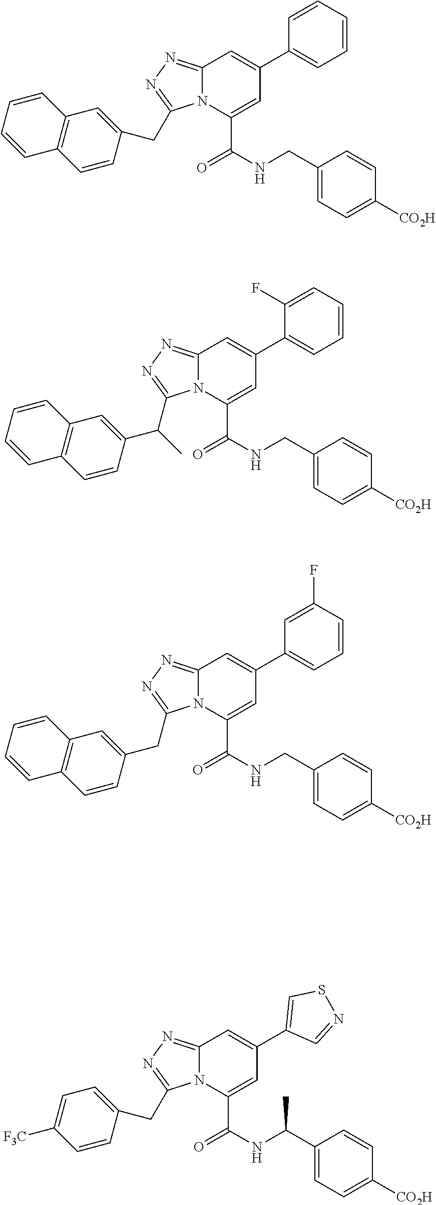 [SMILES: O=C(O)c1ccc(CNC(=O)c2cc(-c3ccccc3)cc3nnc(Cc4ccc5ccccc5c4)n23)cc1 ; CC(c1ccc2ccccc2c1)c1nnc2cc(-c3ccccc3F)cc(C(=O)NCc3ccc(C(=O)O)cc3)n12 ; O=C(O)c1ccc(CNC(=O)c2cc(-c3cccc(F)c3)cc3nnc(Cc4ccc5ccccc5c4)n23)cc1 ; C[C@H](NC(=O)c1cc(-c2cnsc2)cc2nnc(Cc3ccc(C(F)(F)F)cc3)n12)c1ccc(C(=O)O)cc1]

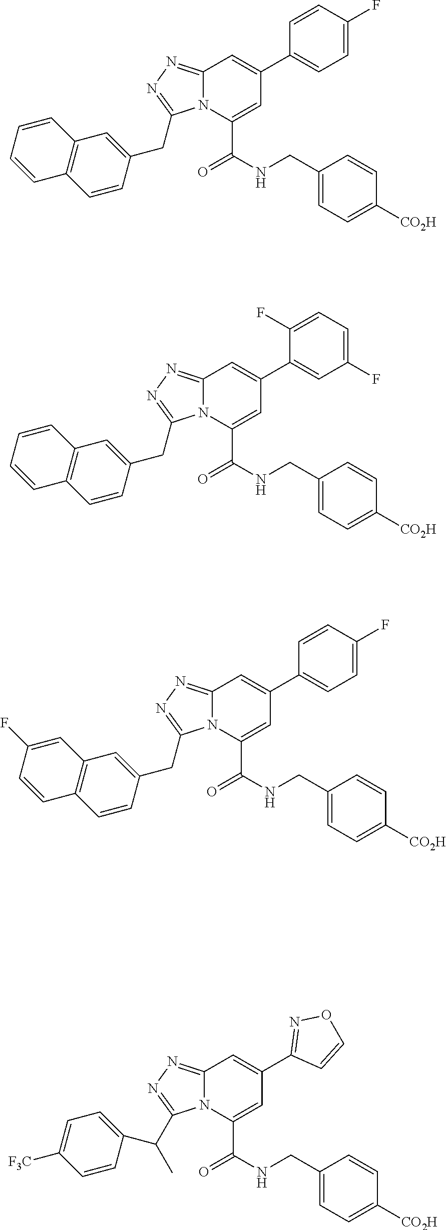 [SMILES: O=C(O)c1ccc(CNC(=O)c2cc(-c3ccc(F)cc3)cc3nnc(Cc4ccc5ccccc5c4)n23)cc1 ; O=C(O)c1ccc(CNC(=O)c2cc(-c3cc(F)ccc3F)cc3nnc(Cc4ccc5ccccc5c4)n23)cc1 ; O=C(O)c1ccc(CNC(=O)c2cc(-c3ccc(F)cc3)cc3nnc(Cc4ccc5ccc(F)cc5c4)n23)cc1 ; CC(c1ccc(C(F)(F)F)cc1)c1nnc2cc(-c3ccon3)cc(C(=O)NCc3ccc(C(=O)O)cc3)n12]

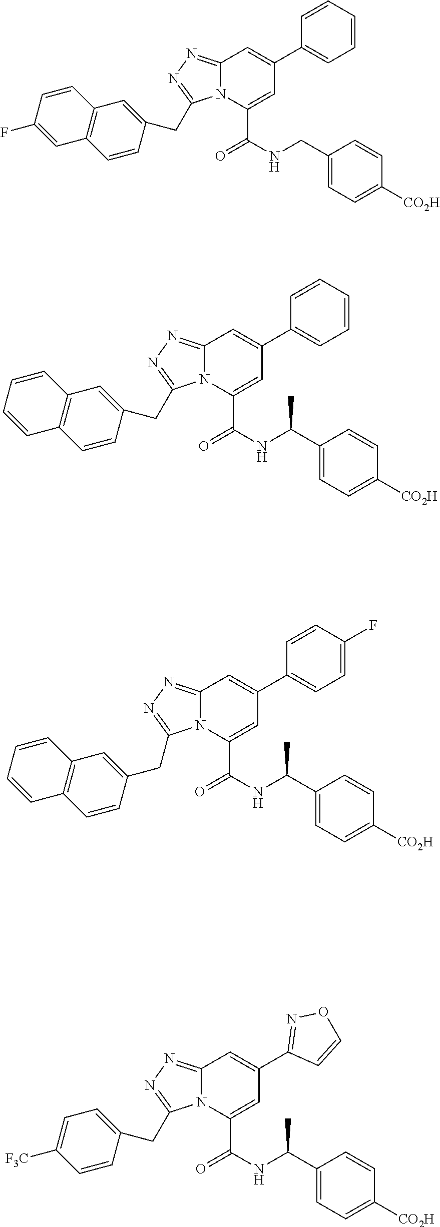 [SMILES: O=C(O)c1ccc(CNC(=O)c2cc(-c3ccccc3)cc3nnc(Cc4ccc5cc(F)ccc5c4)n23)cc1 ; C[C@H](NC(=O)c1cc(-c2ccccc2)cc2nnc(Cc3ccc4ccccc4c3)n12)c1ccc(C(=O)O)cc1 ; C[C@H](NC(=O)c1cc(-c2ccc(F)cc2)cc2nnc(Cc3ccc4ccccc4c3)n12)c1ccc(C(=O)O)cc1 ; C[C@H](NC(=O)c1cc(-c2ccon2)cc2nnc(Cc3ccc(C(F)(F)F)cc3)n12)c1ccc(C(=O)O)cc1]

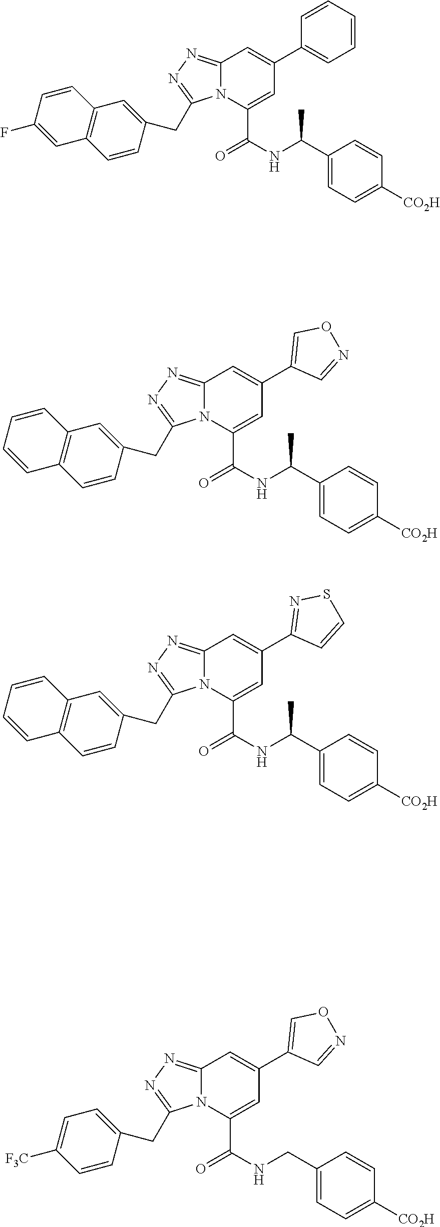 [SMILES: C[C@H](NC(=O)c1cc(-c2ccccc2)cc2nnc(Cc3ccc4cc(F)ccc4c3)n12)c1ccc(C(=O)O)cc1 ; C[C@H](NC(=O)c1cc(-c2cnoc2)cc2nnc(Cc3ccc4ccccc4c3)n12)c1ccc(C(=O)O)cc1 ; C[C@H](NC(=O)c1cc(-c2ccsn2)cc2nnc(Cc3ccc4ccccc4c3)n12)c1ccc(C(=O)O)cc1 ; O=C(O)c1ccc(CNC(=O)c2cc(-c3cnoc3)cc3nnc(Cc4ccc(C(F)(F)F)cc4)n23)cc1]

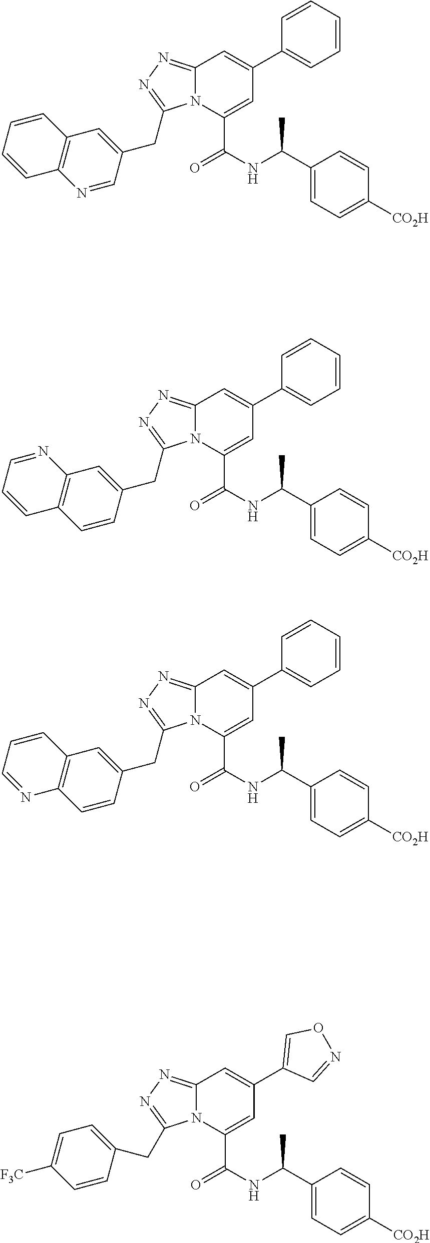 [SMILES: C[C@H](NC(=O)c1cc(-c2ccccc2)cc2nnc(Cc3cnc4ccccc4c3)n12)c1ccc(C(=O)O)cc1 ; C[C@H](NC(=O)c1cc(-c2ccccc2)cc2nnc(Cc3ccc4cccnc4c3)n12)c1ccc(C(=O)O)cc1 ; C[C@H](NC(=O)c1cc(-c2ccccc2)cc2nnc(Cc3ccc4ncccc4c3)n12)c1ccc(C(=O)O)cc1 ; C[C@H](NC(=O)c1cc(-c2cnoc2)cc2nnc(Cc3ccc(C(F)(F)F)cc3)n12)c1ccc(C(=O)O)cc1]

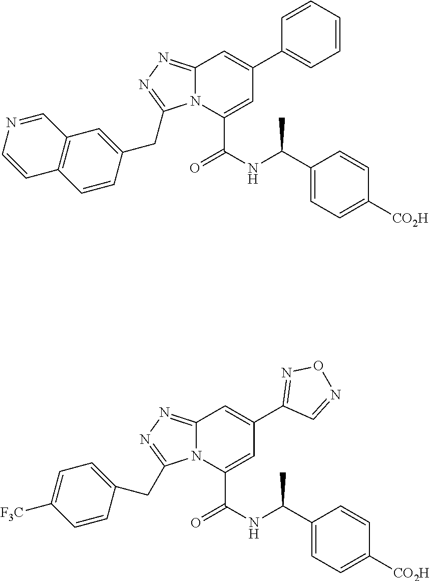 [SMILES: C[C@H](NC(=O)c1cc(-c2ccccc2)cc2nnc(Cc3ccc4ccncc4c3)n12)c1ccc(C(=O)O)cc1 ; C[C@H](NC(=O)c1cc(-c2cnon2)cc2nnc(Cc3ccc(C(F)(F)F)cc3)n12)c1ccc(C(=O)O)cc1]

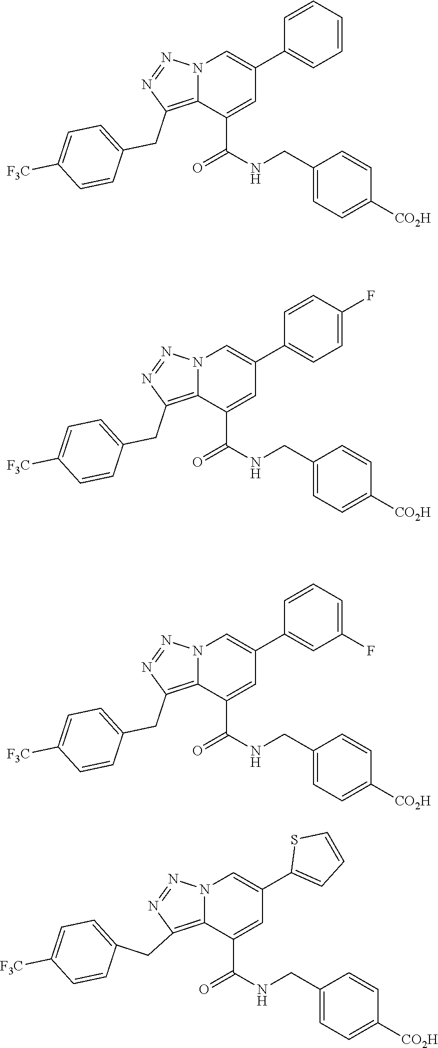

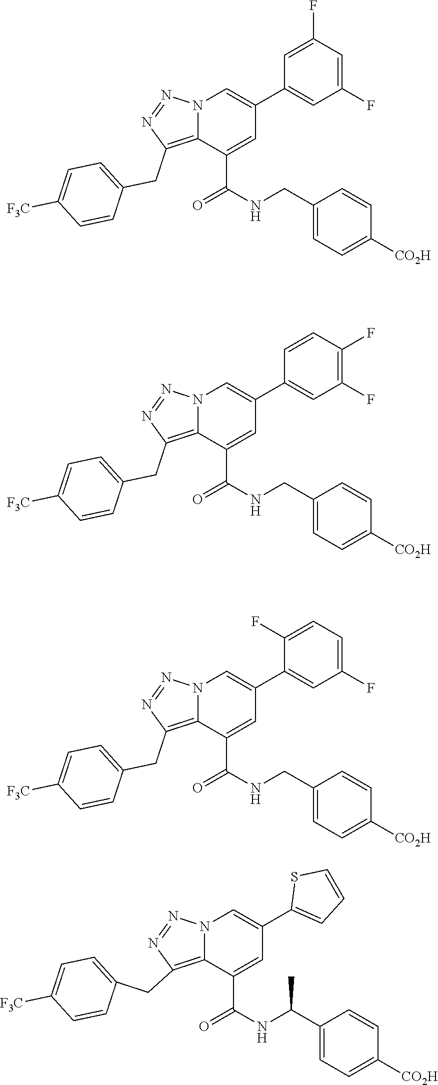

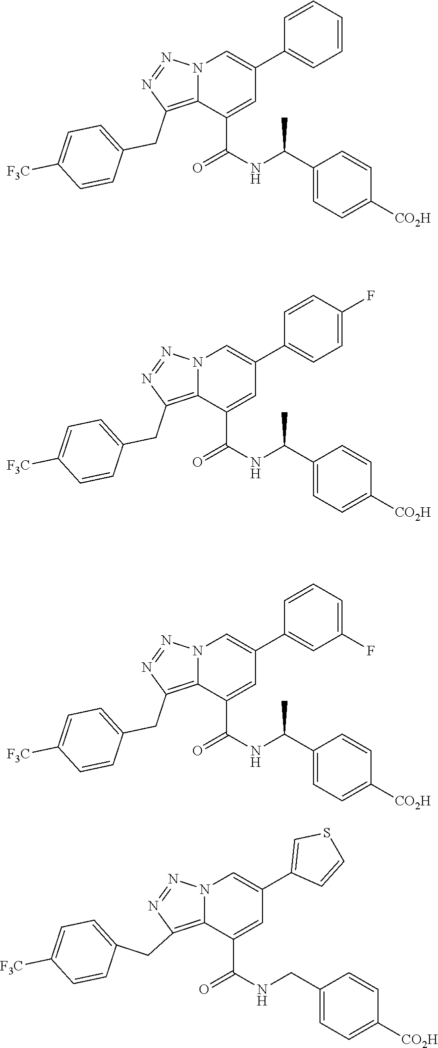

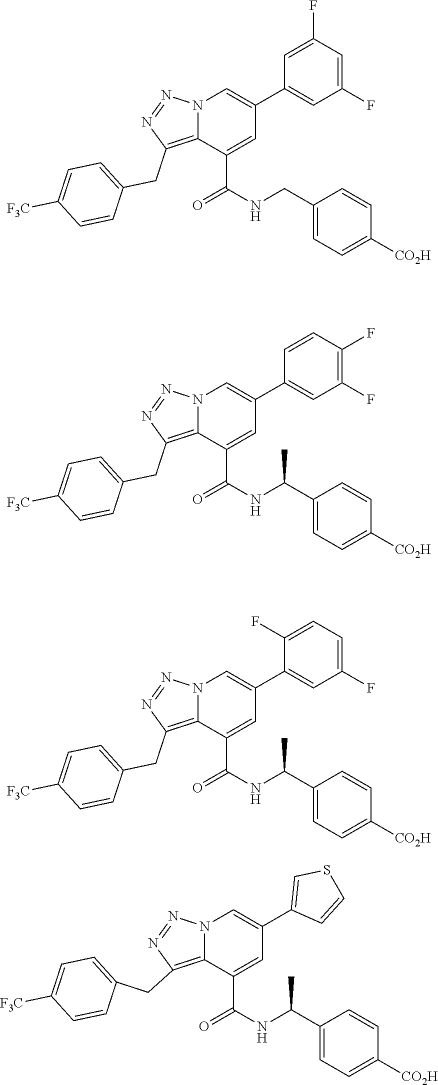

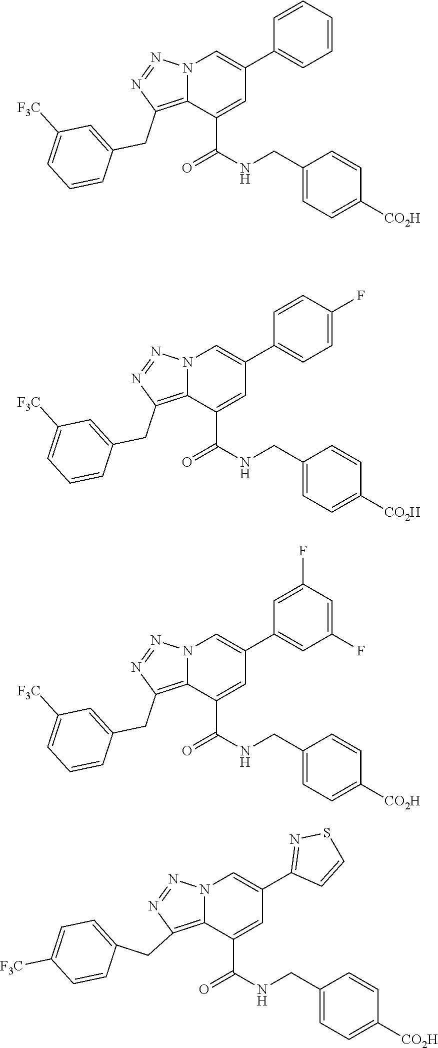

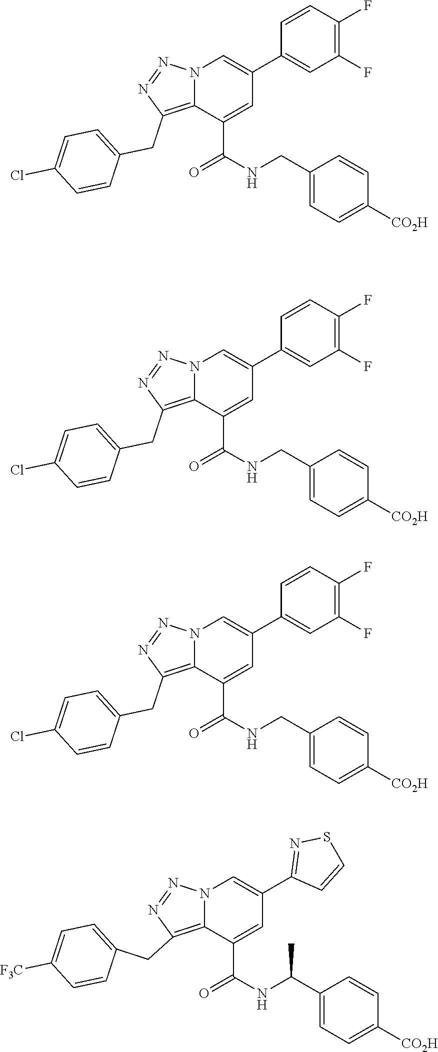 [SMILES: O=C(O)c1ccc(CNC(=O)c2cc(-c3ccc(F)c(F)c3)cn3nnc(Cc4ccc(Cl)cc4)c23)cc1 ; O=C(O)c1ccc(CNC(=O)c2cc(-c3ccc(F)c(F)c3)cn3nnc(Cc4ccc(Cl)cc4)c23)cc1 ; O=C(O)c1ccc(CNC(=O)c2cc(-c3ccc(F)c(F)c3)cn3nnc(Cc4ccc(Cl)cc4)c23)cc1 ; C[C@H](NC(=O)c1cc(-c2ccsn2)cn2nnc(Cc3ccc(C(F)(F)F)cc3)c12)c1ccc(C(=O)O)cc1]

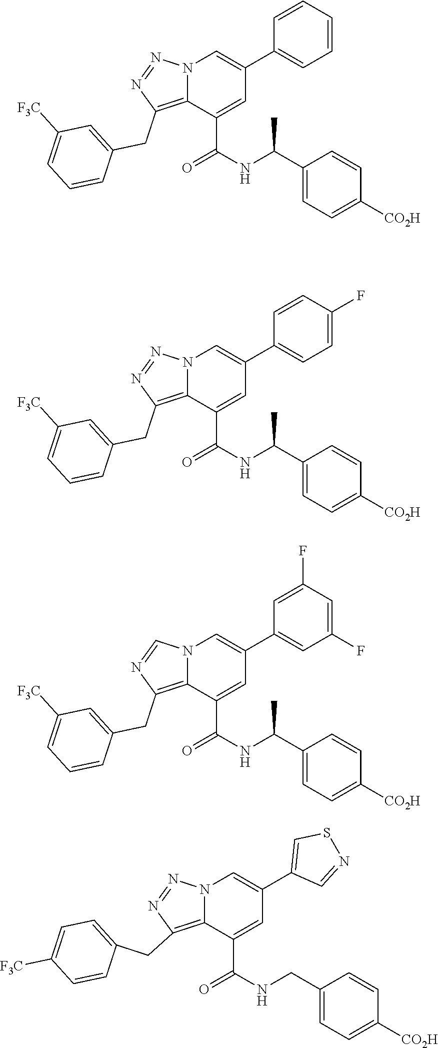

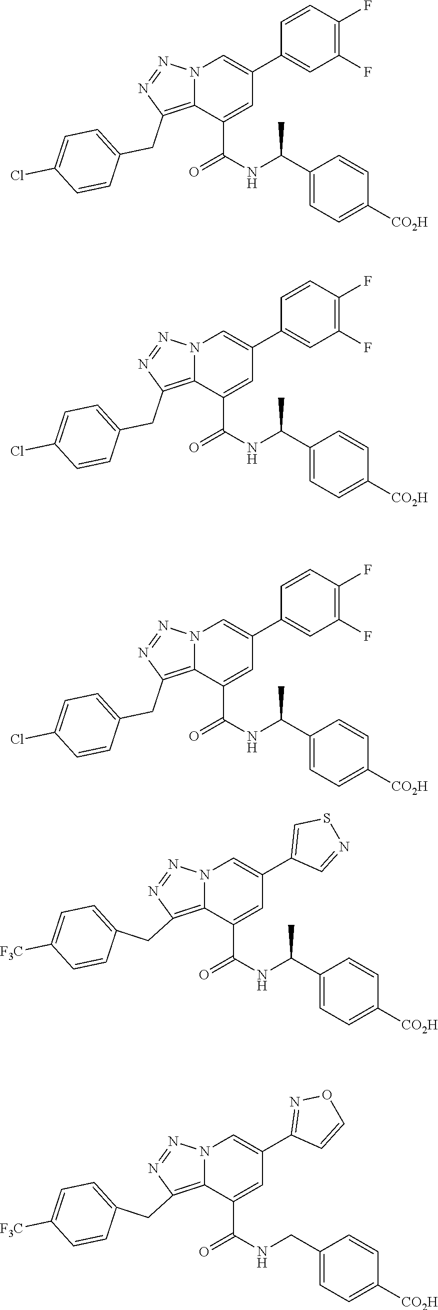

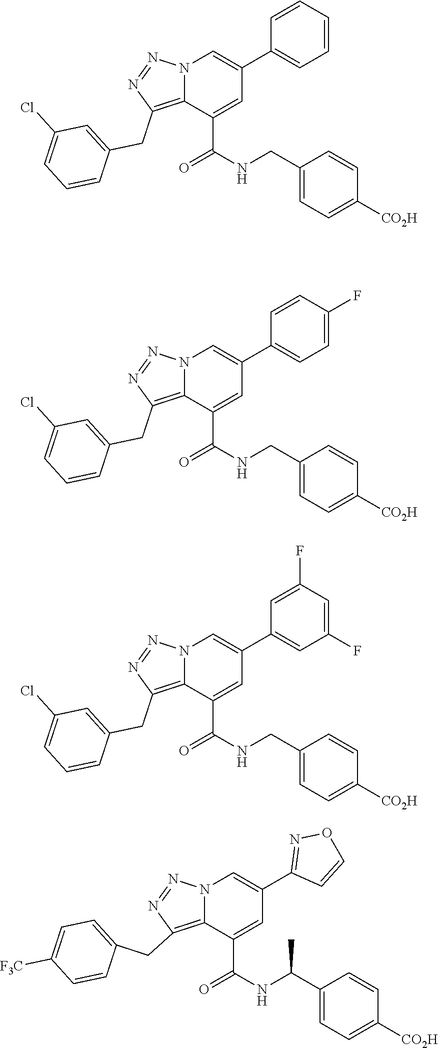

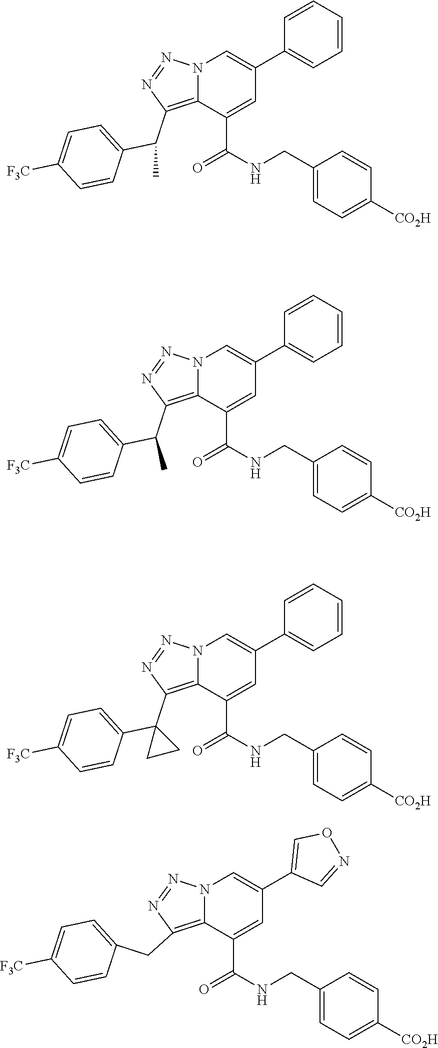

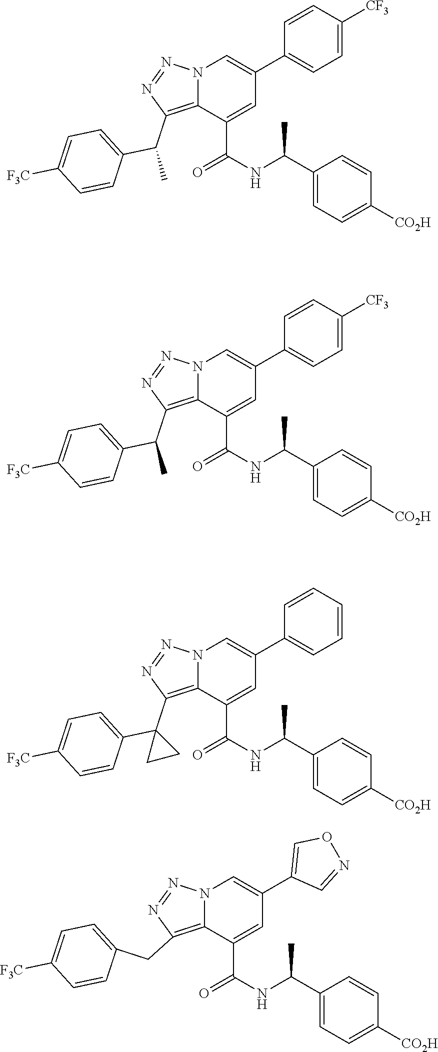

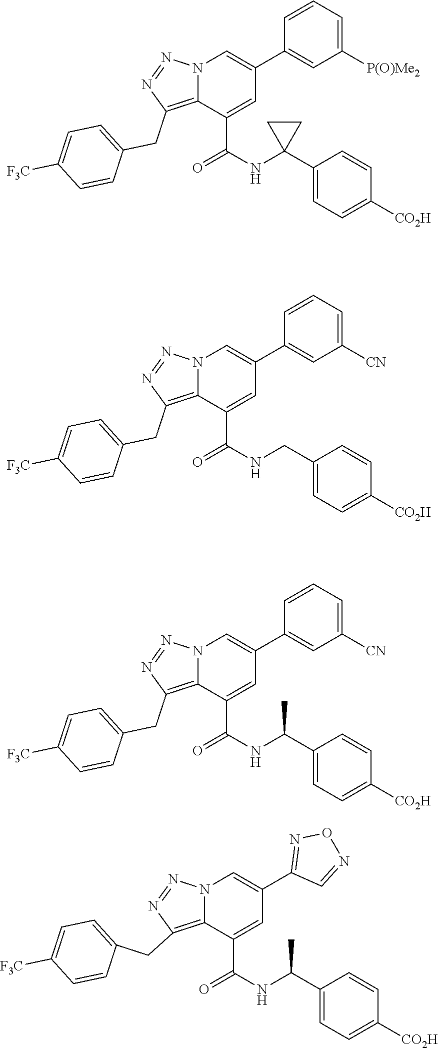

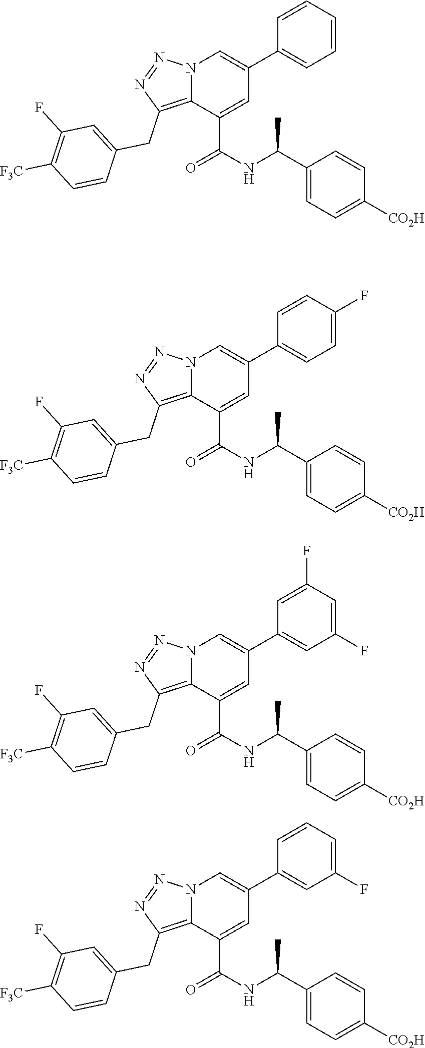

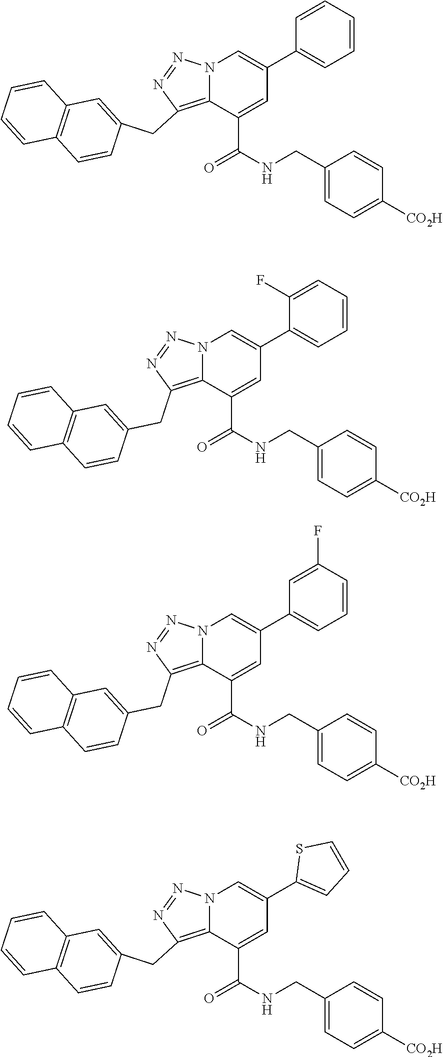

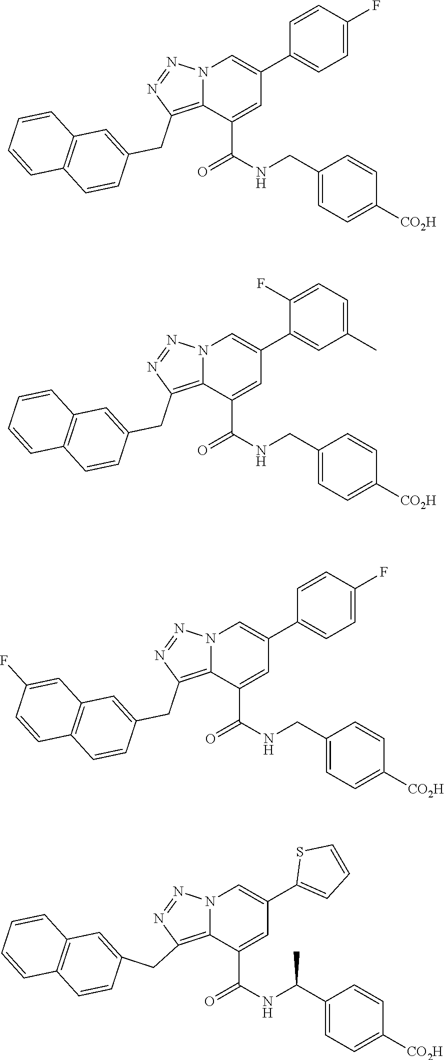 [SMILES: O=C(O)c1ccc(CNC(=O)c2cc(-c3ccc(F)cc3)cn3nnc(Cc4ccc5ccccc5c4)c23)cc1 ; Cc1ccc(F)c(-c2cc(C(=O)NCc3ccc(C(=O)O)cc3)c3c(Cc4ccc5ccccc5c4)nnn3c2)c1 ; O=C(O)c1ccc(CNC(=O)c2cc(-c3ccc(F)cc3)cn3nnc(Cc4ccc5ccc(F)cc5c4)c23)cc1 ; C[C@H](NC(=O)c1cc(-c2cccs2)cn2nnc(Cc3ccc4ccccc4c3)c12)c1ccc(C(=O)O)cc1]

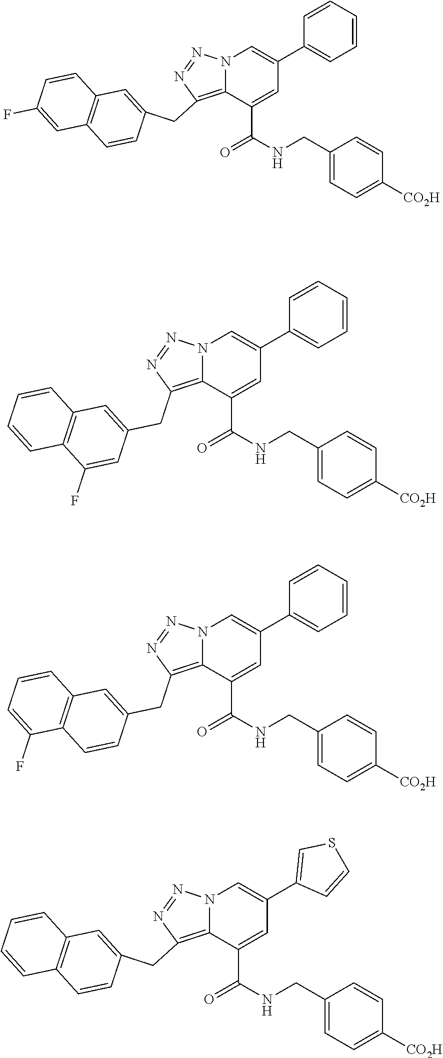 [SMILES: O=C(O)c1ccc(CNC(=O)c2cc(-c3ccccc3)cn3nnc(Cc4ccc5cc(F)ccc5c4)c23)cc1 ; O=C(O)c1ccc(CNC(=O)c2cc(-c3ccccc3)cn3nnc(Cc4cc(F)c5ccccc5c4)c23)cc1 ; O=C(O)c1ccc(CNC(=O)c2cc(-c3ccccc3)cn3nnc(Cc4ccc5c(F)cccc5c4)c23)cc1 ; O=C(O)c1ccc(CNC(=O)c2cc(-c3ccsc3)cn3nnc(Cc4ccc5ccccc5c4)c23)cc1]

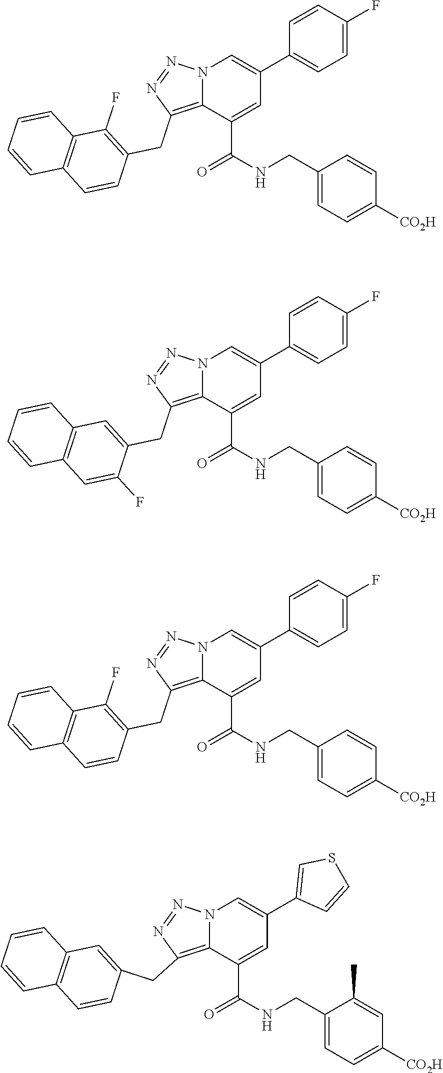

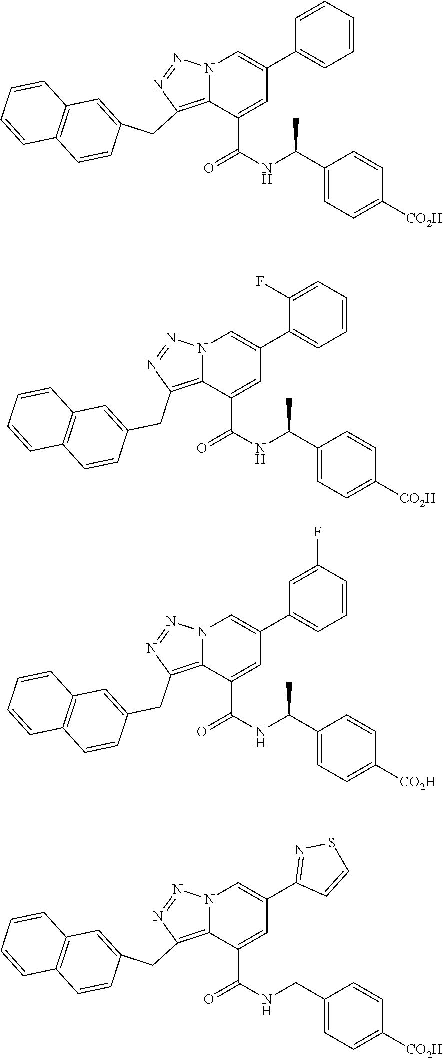

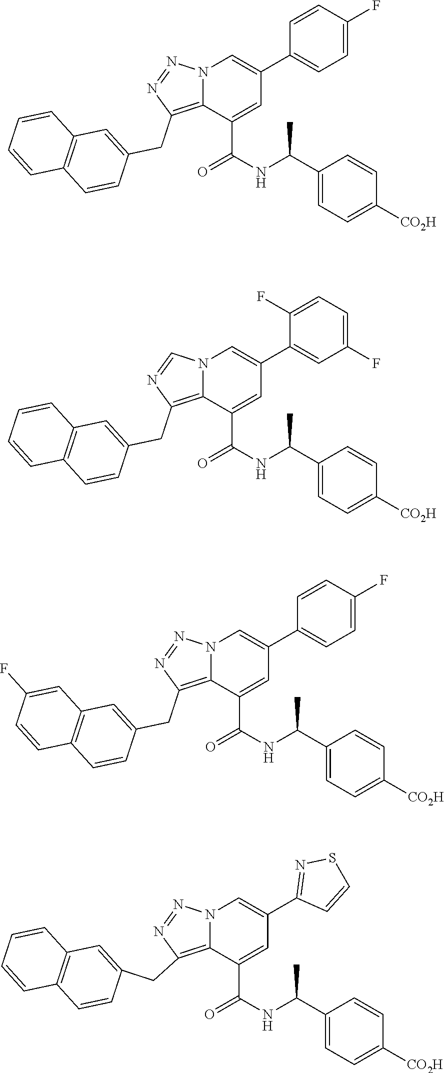 [SMILES: C[C@H](NC(=O)c1cc(-c2ccc(F)cc2)cn2nnc(Cc3ccc4ccccc4c3)c12)c1ccc(C(=O)O)cc1 ; C[C@H](NC(=O)c1cc(-c2cc(F)ccc2F)cn2cnc(Cc3ccc4ccccc4c3)c12)c1ccc(C(=O)O)cc1 ; C[C@H](NC(=O)c1cc(-c2ccc(F)cc2)cn2nnc(Cc3ccc4ccc(F)cc4c3)c12)c1ccc(C(=O)O)cc1 ; C[C@H](NC(=O)c1cc(-c2ccsn2)cn2nnc(Cc3ccc4ccccc4c3)c12)c1ccc(C(=O)O)cc1]

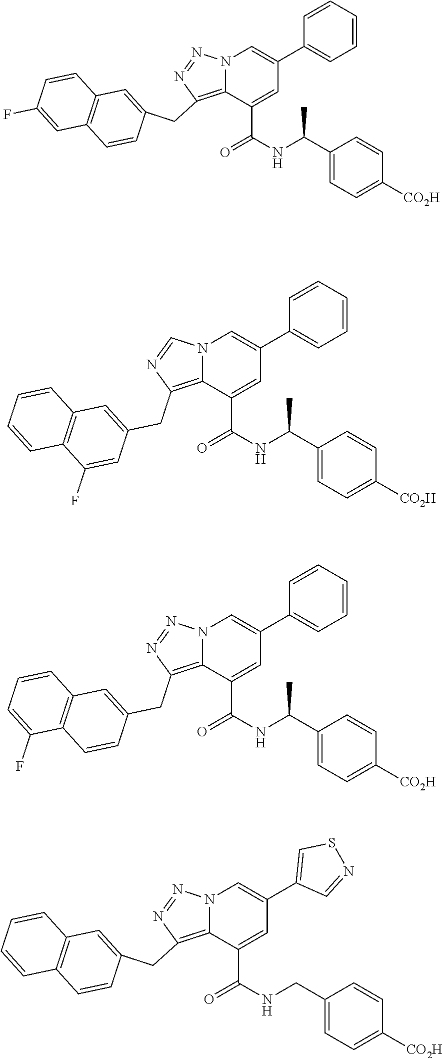 [SMILES: C[C@H](NC(=O)c1cc(-c2ccccc2)cn2nnc(Cc3ccc4cc(F)ccc4c3)c12)c1ccc(C(=O)O)cc1 ; C[C@H](NC(=O)c1cc(-c2ccccc2)cn2cnc(Cc3cc(F)c4ccccc4c3)c12)c1ccc(C(=O)O)cc1 ; C[C@H](NC(=O)c1cc(-c2ccccc2)cn2nnc(Cc3ccc4c(F)cccc4c3)c12)c1ccc(C(=O)O)cc1 ; O=C(O)c1ccc(CNC(=O)c2cc(-c3cnsc3)cn3nnc(Cc4ccc5ccccc5c4)c23)cc1]

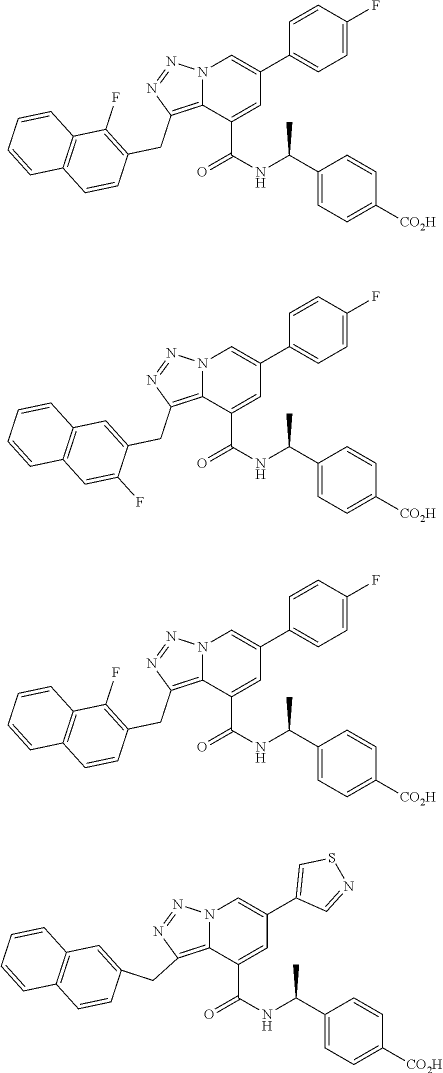 [SMILES: C[C@H](NC(=O)c1cc(-c2ccc(F)cc2)cn2nnc(Cc3ccc4ccccc4c3F)c12)c1ccc(C(=O)O)cc1 ; C[C@H](NC(=O)c1cc(-c2ccc(F)cc2)cn2nnc(Cc3cc4ccccc4cc3F)c12)c1ccc(C(=O)O)cc1 ; C[C@H](NC(=O)c1cc(-c2ccc(F)cc2)cn2nnc(Cc3ccc4ccccc4c3F)c12)c1ccc(C(=O)O)cc1 ; C[C@H](NC(=O)c1cc(-c2cnsc2)cn2nnc(Cc3ccc4ccccc4c3)c12)c1ccc(C(=O)O)cc1]

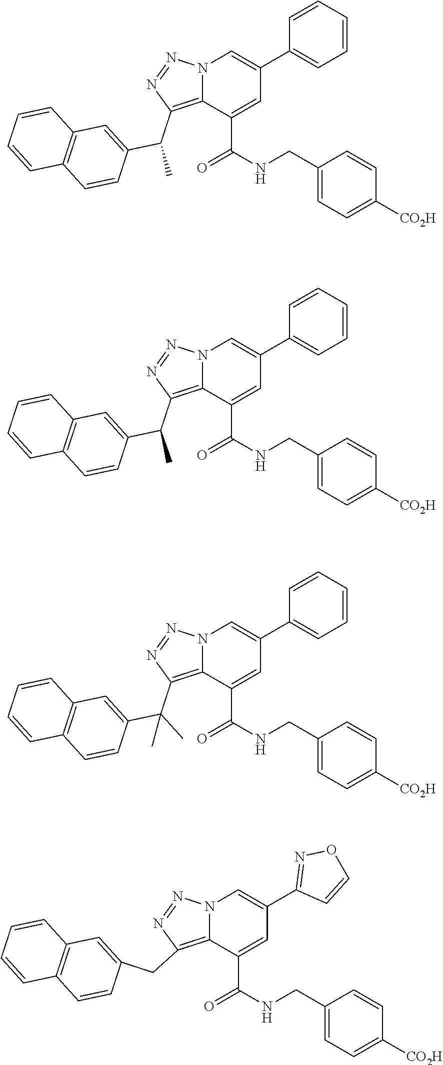 [SMILES: C[C@H](c1ccc2ccccc2c1)c1nnn2cc(-c3ccccc3)cc(C(=O)NCc3ccc(C(=O)O)cc3)c12 ; C[C@@H](c1ccc2ccccc2c1)c1nnn2cc(-c3ccccc3)cc(C(=O)NCc3ccc(C(=O)O)cc3)c12 ; CC(C)(c1ccc2ccccc2c1)c1nnn2cc(-c3ccccc3)cc(C(=O)NCc3ccc(C(=O)O)cc3)c12 ; O=C(O)c1ccc(CNC(=O)c2cc(-c3ccon3)cn3nnc(Cc4ccc5ccccc5c4)c23)cc1]

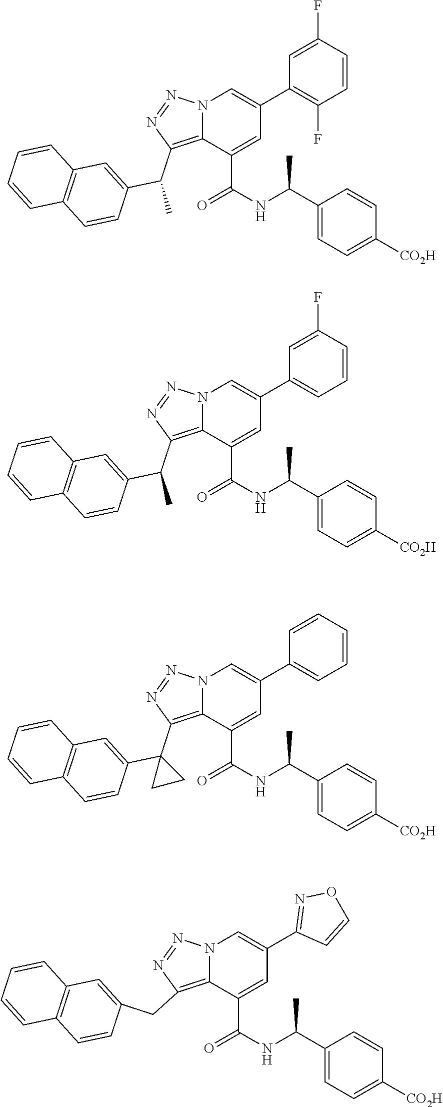 [SMILES: C[C@H](c1ccc2ccccc2c1)c1nnn2cc(-c3cc(F)ccc3F)cc(C(=O)N[C@@H](C)c3ccc(C(=O)O)cc3)c12 ; C[C@@H](c1ccc2ccccc2c1)c1nnn2cc(-c3cccc(F)c3)cc(C(=O)N[C@@H](C)c3ccc(C(=O)O)cc3)c12 ; C[C@H](NC(=O)c1cc(-c2ccccc2)cn2nnc(C3(c4ccc5ccccc5c4)CC3)c12)c1ccc(C(=O)O)cc1 ; C[C@H](NC(=O)c1cc(-c2ccon2)cn2nnc(Cc3ccc4ccccc4c3)c12)c1ccc(C(=O)O)cc1]

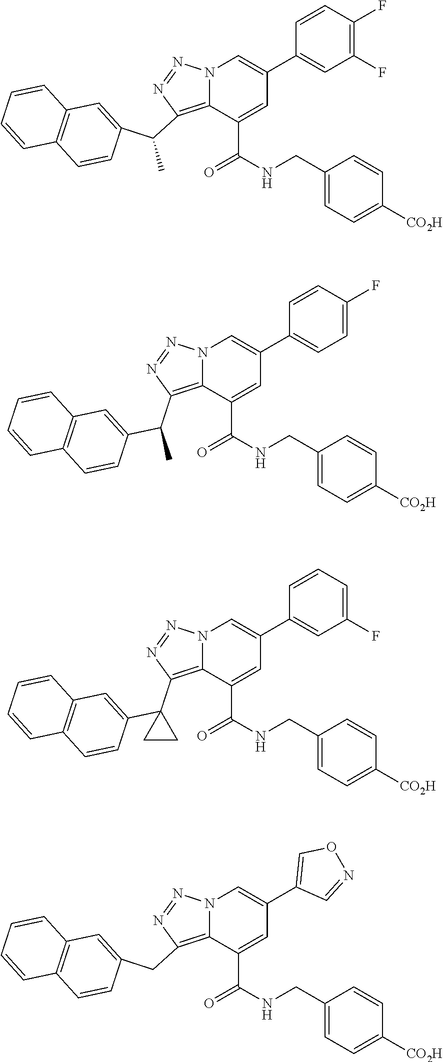 [SMILES: C[C@H](c1ccc2ccccc2c1)c1nnn2cc(-c3ccc(F)c(F)c3)cc(C(=O)NCc3ccc(C(=O)O)cc3)c12 ; C[C@@H](c1ccc2ccccc2c1)c1nnn2cc(-c3ccc(F)cc3)cc(C(=O)NCc3ccc(C(=O)O)cc3)c12 ; O=C(O)c1ccc(CNC(=O)c2cc(-c3cccc(F)c3)cn3nnc(C4(c5ccc6ccccc6c5)CC4)c23)cc1 ; O=C(O)c1ccc(CNC(=O)c2cc(-c3cnoc3)cn3nnc(Cc4ccc5ccccc5c4)c23)cc1]

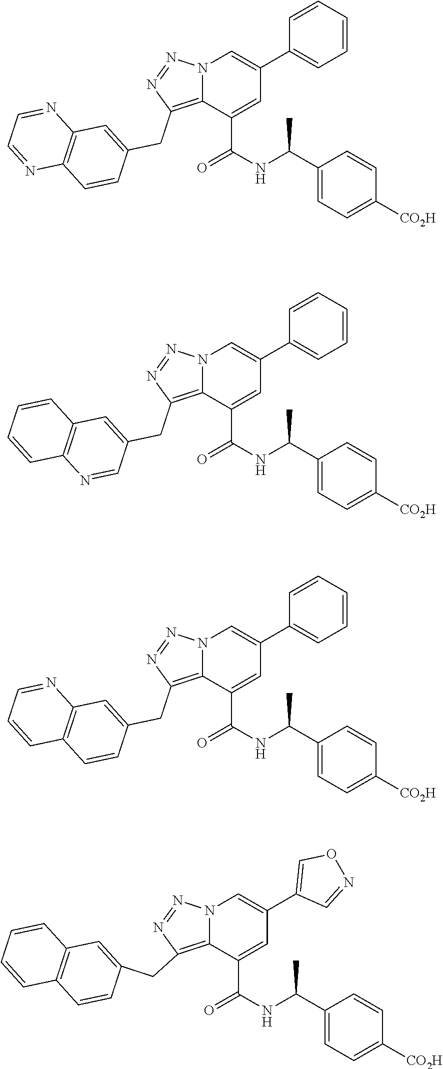 [SMILES: C[C@H](NC(=O)c1cc(-c2ccccc2)cn2nnc(Cc3ccc4nccnc4c3)c12)c1ccc(C(=O)O)cc1 ; C[C@H](NC(=O)c1cc(-c2ccccc2)cn2nnc(Cc3cnc4ccccc4c3)c12)c1ccc(C(=O)O)cc1 ; C[C@H](NC(=O)c1cc(-c2ccccc2)cn2nnc(Cc3ccc4cccnc4c3)c12)c1ccc(C(=O)O)cc1 ; C[C@H](NC(=O)c1cc(-c2cnoc2)cn2nnc(Cc3ccc4ccccc4c3)c12)c1ccc(C(=O)O)cc1]

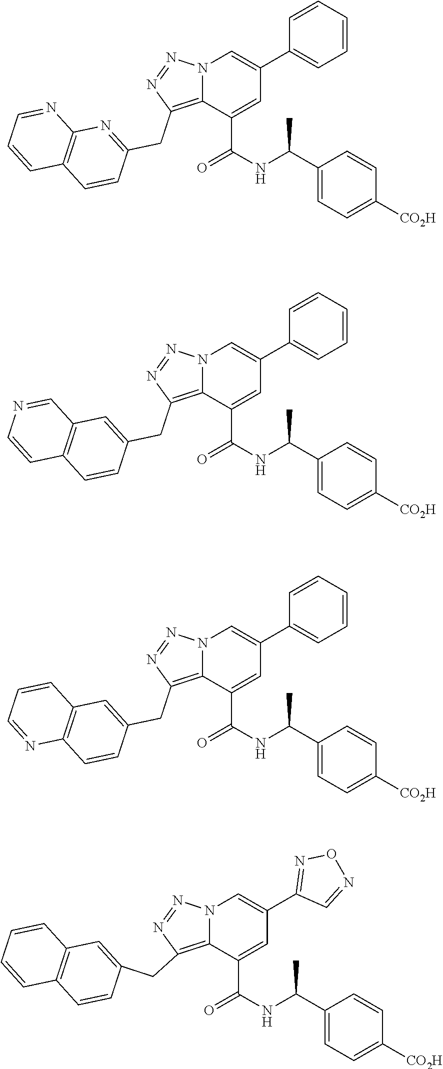 [SMILES: C[C@H](NC(=O)c1cc(-c2ccccc2)cn2nnc(Cc3ccc4cccnc4n3)c12)c1ccc(C(=O)O)cc1 ; C[C@H](NC(=O)c1cc(-c2ccccc2)cn2nnc(Cc3ccc4ccncc4c3)c12)c1ccc(C(=O)O)cc1 ; C[C@H](NC(=O)c1cc(-c2ccccc2)cn2nnc(Cc3ccc4ncccc4c3)c12)c1ccc(C(=O)O)cc1 ; C[C@H](NC(=O)c1cc(-c2cnon2)cn2nnc(Cc3ccc4ccccc4c3)c12)c1ccc(C(=O)O)cc1]

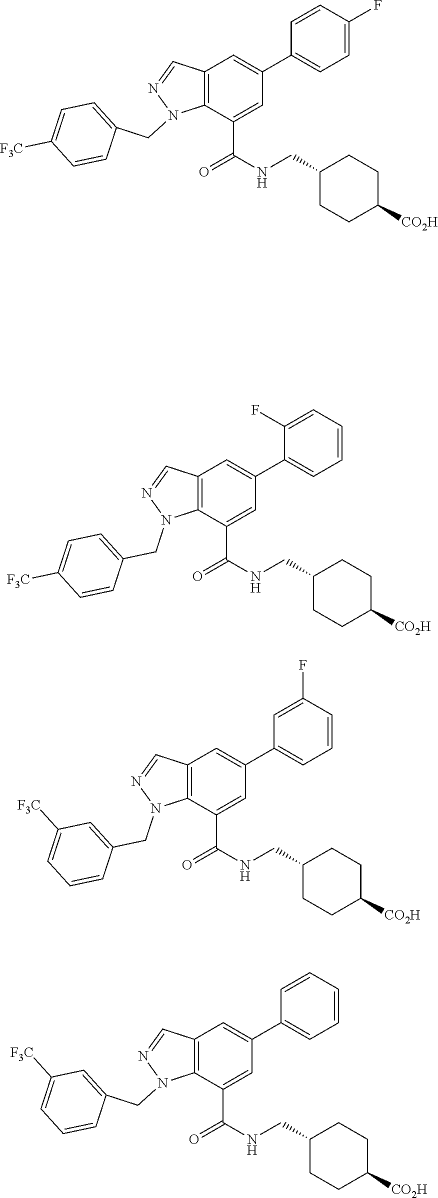 [SMILES: O=C(NC[C@H]1CC[C@H](C(=O)O)CC1)c1cc(-c2ccc(F)cc2)cc2cnn(Cc3ccc(C(F)(F)F)cc3)c12 ; O=C(NC[C@H]1CC[C@H](C(=O)O)CC1)c1cc(-c2ccccc2F)cc2cnn(Cc3ccc(C(F)(F)F)cc3)c12 ; O=C(NC[C@H]1CC[C@H](C(=O)O)CC1)c1cc(-c2cccc(F)c2)cc2cnn(Cc3cccc(C(F)(F)F)c3)c12 ; O=C(NC[C@H]1CC[C@H](C(=O)O)CC1)c1cc(-c2ccccc2)cc2cnn(Cc3cccc(C(F)(F)F)c3)c12]

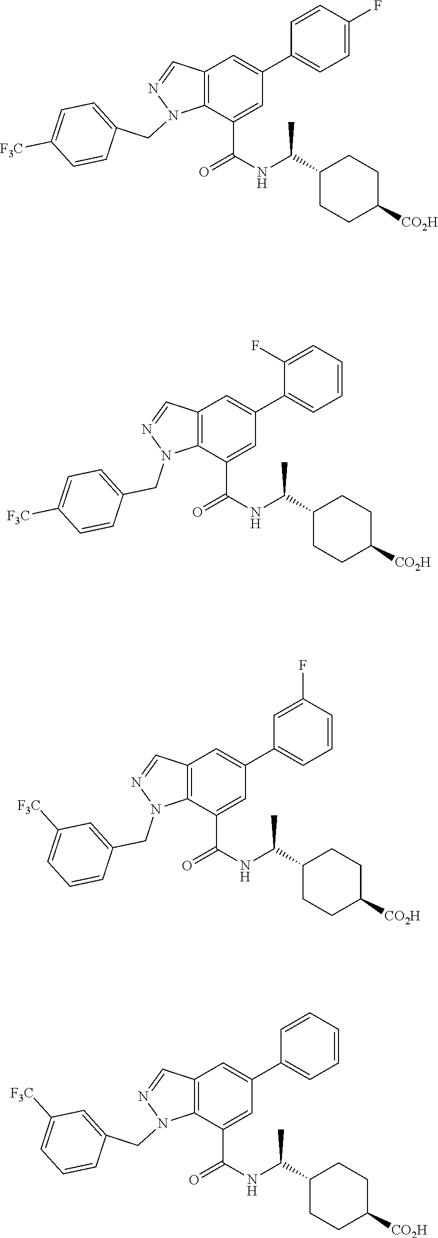 [SMILES: C[C@H](NC(=O)c1cc(-c2ccc(F)cc2)cc2cnn(Cc3ccc(C(F)(F)F)cc3)c12)[C@H]1CC[C@H](C(=O)O)CC1 ; C[C@H](NC(=O)c1cc(-c2ccccc2F)cc2cnn(Cc3ccc(C(F)(F)F)cc3)c12)[C@H]1CC[C@H](C(=O)O)CC1 ; C[C@H](NC(=O)c1cc(-c2cccc(F)c2)cc2cnn(Cc3cccc(C(F)(F)F)c3)c12)[C@H]1CC[C@H](C(=O)O)CC1 ; C[C@H](NC(=O)c1cc(-c2ccccc2)cc2cnn(Cc3cccc(C(F)(F)F)c3)c12)[C@H]1CC[C@H](C(=O)O)CC1]

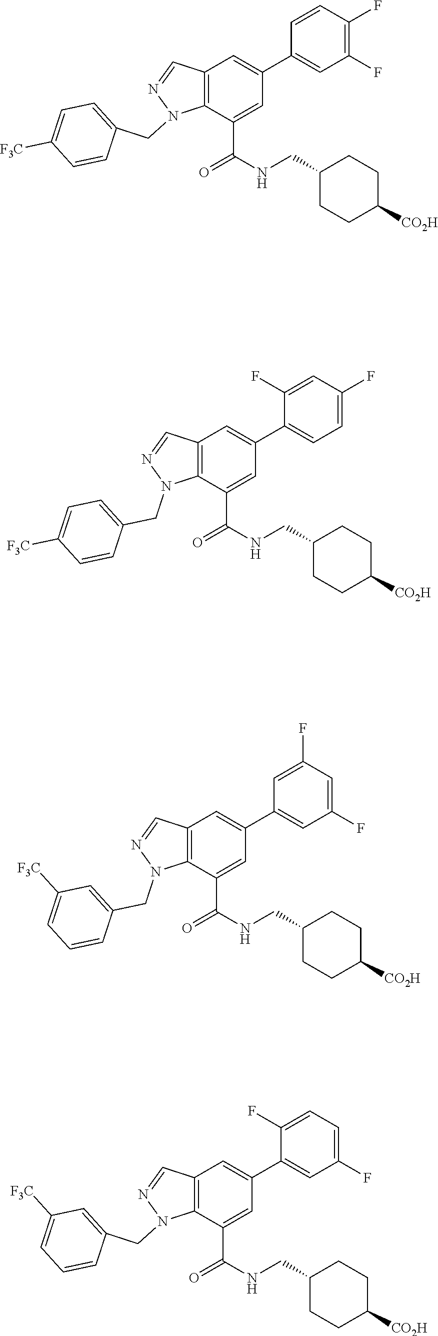 [SMILES: O=C(NC[C@H]1CC[C@H](C(=O)O)CC1)c1cc(-c2ccc(F)c(F)c2)cc2cnn(Cc3ccc(C(F)(F)F)cc3)c12 ; O=C(NC[C@H]1CC[C@H](C(=O)O)CC1)c1cc(-c2ccc(F)cc2F)cc2cnn(Cc3ccc(C(F)(F)F)cc3)c12 ; O=C(NC[C@H]1CC[C@H](C(=O)O)CC1)c1cc(-c2cc(F)cc(F)c2)cc2cnn(Cc3cccc(C(F)(F)F)c3)c12 ; O=C(NC[C@H]1CC[C@H](C(=O)O)CC1)c1cc(-c2cc(F)ccc2F)cc2cnn(Cc3cccc(C(F)(F)F)c3)c12]

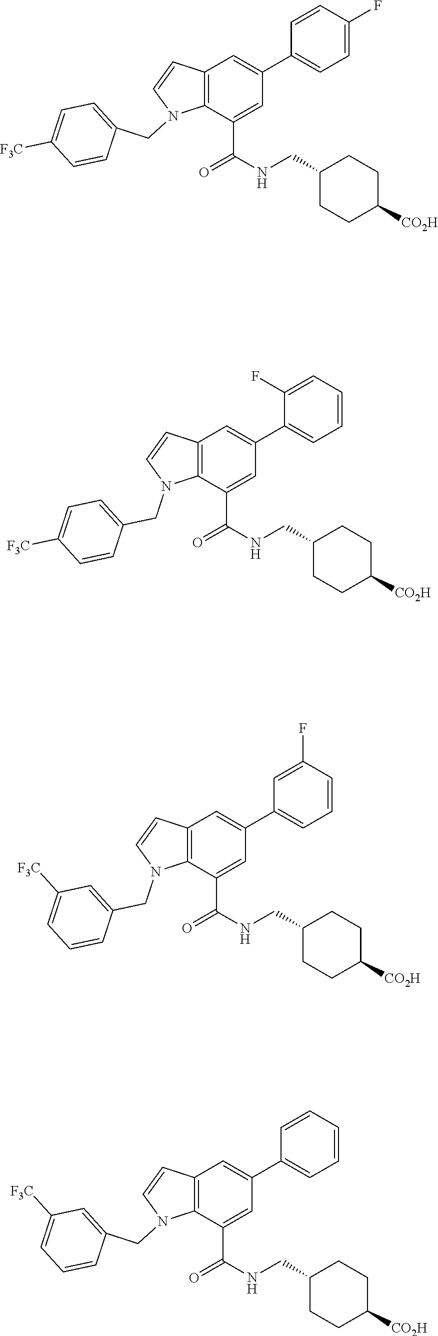 [SMILES: O=C(NC[C@H]1CC[C@H](C(=O)O)CC1)c1cc(-c2ccc(F)cc2)cc2ccn(Cc3ccc(C(F)(F)F)cc3)c12 ; O=C(NC[C@H]1CC[C@H](C(=O)O)CC1)c1cc(-c2ccccc2F)cc2ccn(Cc3ccc(C(F)(F)F)cc3)c12 ; O=C(NC[C@H]1CC[C@H](C(=O)O)CC1)c1cc(-c2cccc(F)c2)cc2ccn(Cc3cccc(C(F)(F)F)c3)c12 ; O=C(NC[C@H]1CC[C@H](C(=O)O)CC1)c1cc(-c2ccccc2)cc2ccn(Cc3cccc(C(F)(F)F)c3)c12]

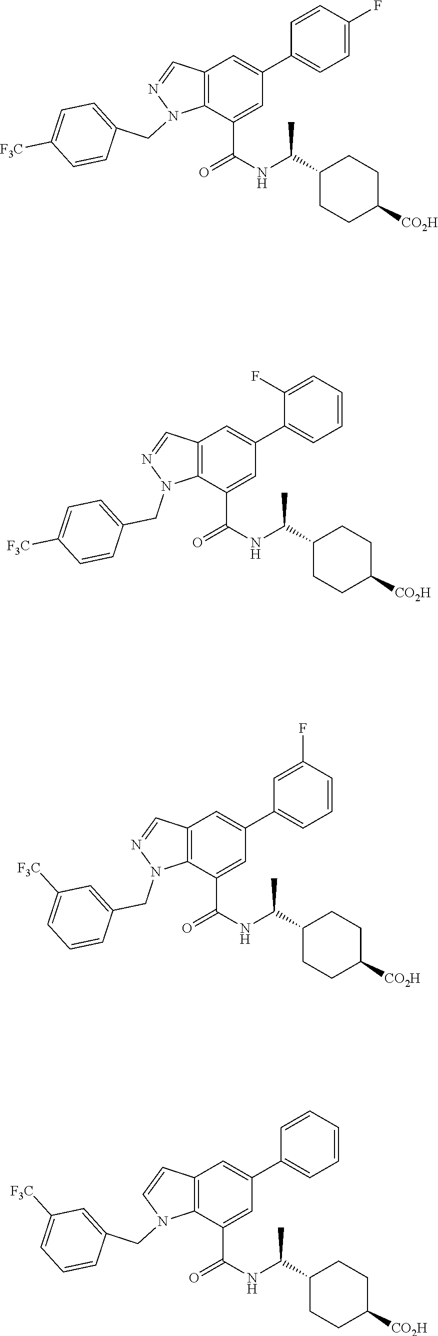 [SMILES: C[C@H](NC(=O)c1cc(-c2ccc(F)cc2)cc2cnn(Cc3ccc(C(F)(F)F)cc3)c12)[C@H]1CC[C@H](C(=O)O)CC1 ; C[C@H](NC(=O)c1cc(-c2ccccc2F)cc2cnn(Cc3ccc(C(F)(F)F)cc3)c12)[C@H]1CC[C@H](C(=O)O)CC1 ; C[C@H](NC(=O)c1cc(-c2cccc(F)c2)cc2cnn(Cc3cccc(C(F)(F)F)c3)c12)[C@H]1CC[C@H](C(=O)O)CC1 ; C[C@H](NC(=O)c1cc(-c2ccccc2)cc2ccn(Cc3cccc(C(F)(F)F)c3)c12)[C@H]1CC[C@H](C(=O)O)CC1]

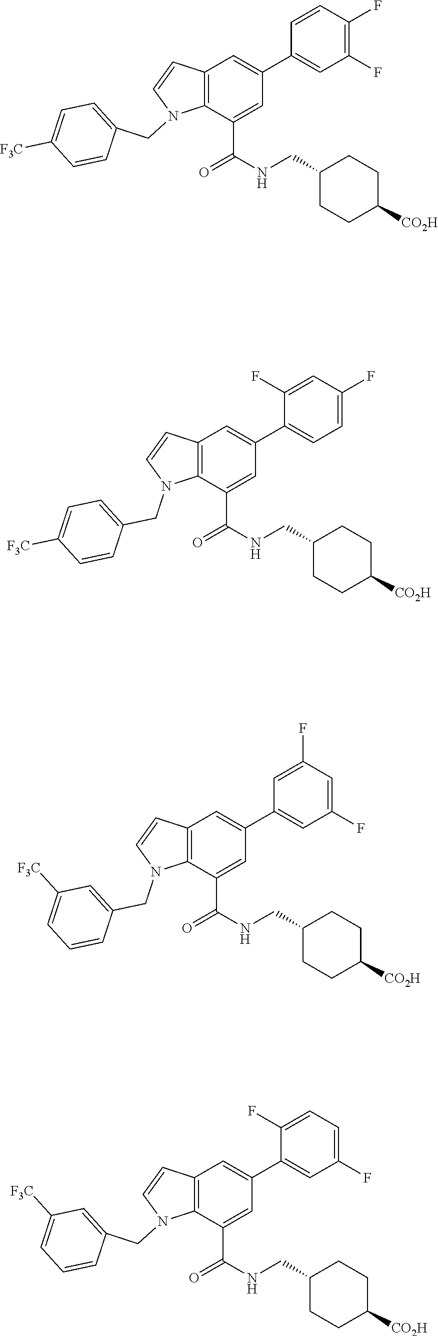 [SMILES: O=C(NC[C@H]1CC[C@H](C(=O)O)CC1)c1cc(-c2ccc(F)c(F)c2)cc2ccn(Cc3ccc(C(F)(F)F)cc3)c12 ; O=C(NC[C@H]1CC[C@H](C(=O)O)CC1)c1cc(-c2ccc(F)cc2F)cc2ccn(Cc3ccc(C(F)(F)F)cc3)c12 ; O=C(NC[C@H]1CC[C@H](C(=O)O)CC1)c1cc(-c2cc(F)cc(F)c2)cc2ccn(Cc3cccc(C(F)(F)F)c3)c12 ; O=C(NC[C@H]1CC[C@H](C(=O)O)CC1)c1cc(-c2cc(F)ccc2F)cc2ccn(Cc3cccc(C(F)(F)F)c3)c12]

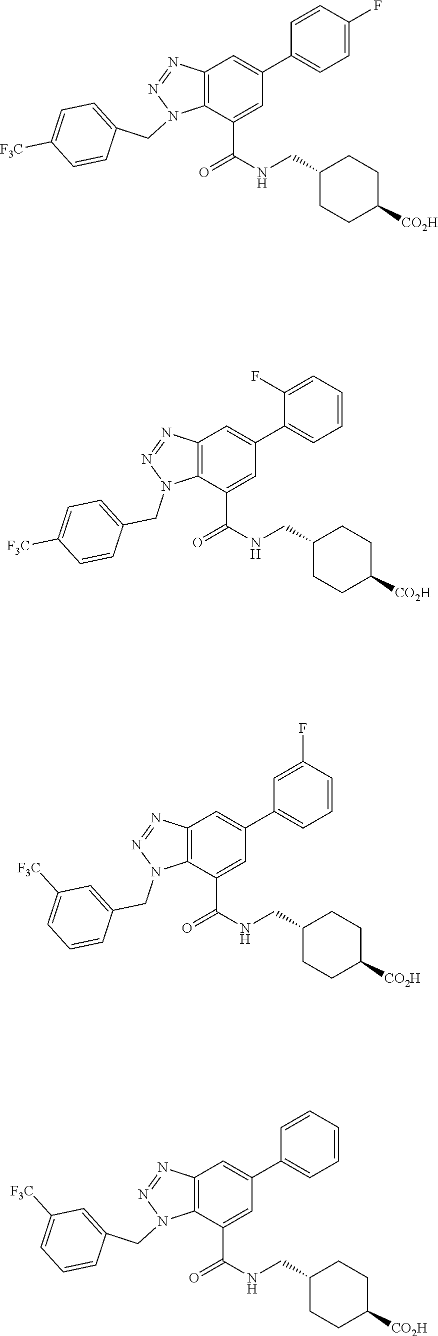 [SMILES: O=C(NC[C@H]1CC[C@H](C(=O)O)CC1)c1cc(-c2ccc(F)cc2)cc2nnn(Cc3ccc(C(F)(F)F)cc3)c12 ; O=C(NC[C@H]1CC[C@H](C(=O)O)CC1)c1cc(-c2ccccc2F)cc2nnn(Cc3ccc(C(F)(F)F)cc3)c12 ; O=C(NC[C@H]1CC[C@H](C(=O)O)CC1)c1cc(-c2cccc(F)c2)cc2nnn(Cc3cccc(C(F)(F)F)c3)c12 ; O=C(NC[C@H]1CC[C@H](C(=O)O)CC1)c1cc(-c2ccccc2)cc2nnn(Cc3cccc(C(F)(F)F)c3)c12]

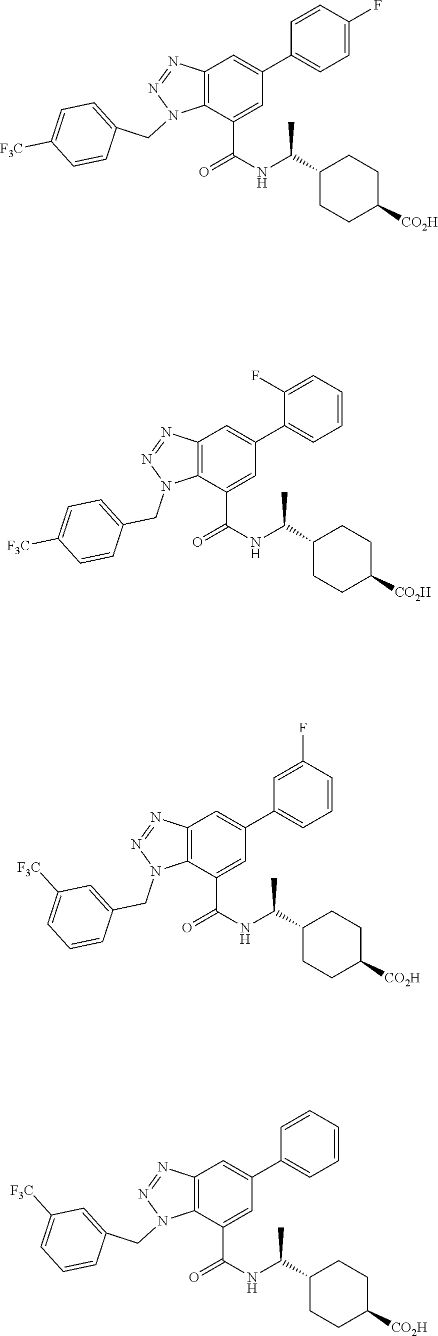 [SMILES: C[C@H](NC(=O)c1cc(-c2ccc(F)cc2)cc2nnn(Cc3ccc(C(F)(F)F)cc3)c12)[C@H]1CC[C@H](C(=O)O)CC1 ; C[C@H](NC(=O)c1cc(-c2ccccc2F)cc2nnn(Cc3ccc(C(F)(F)F)cc3)c12)[C@H]1CC[C@H](C(=O)O)CC1 ; C[C@H](NC(=O)c1cc(-c2cccc(F)c2)cc2nnn(Cc3cccc(C(F)(F)F)c3)c12)[C@H]1CC[C@H](C(=O)O)CC1 ; C[C@H](NC(=O)c1cc(-c2ccccc2)cc2nnn(Cc3cccc(C(F)(F)F)c3)c12)[C@H]1CC[C@H](C(=O)O)CC1]

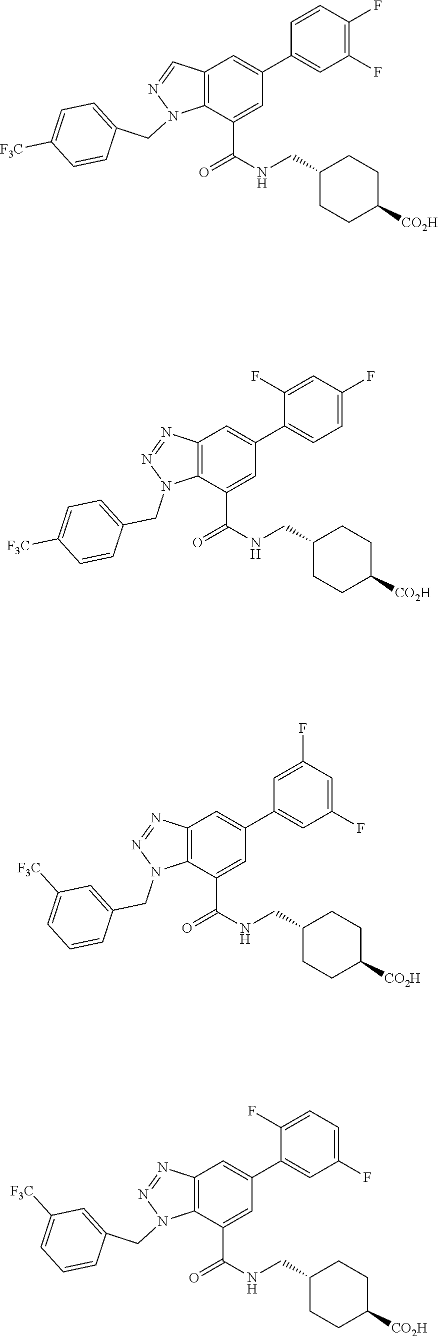 [SMILES: O=C(NC[C@H]1CC[C@H](C(=O)O)CC1)c1cc(-c2ccc(F)c(F)c2)cc2cnn(Cc3ccc(C(F)(F)F)cc3)c12 ; O=C(NC[C@H]1CC[C@H](C(=O)O)CC1)c1cc(-c2ccc(F)cc2F)cc2nnn(Cc3ccc(C(F)(F)F)cc3)c12 ; O=C(NC[C@H]1CC[C@H](C(=O)O)CC1)c1cc(-c2cc(F)cc(F)c2)cc2nnn(Cc3cccc(C(F)(F)F)c3)c12 ; O=C(NC[C@H]1CC[C@H](C(=O)O)CC1)c1cc(-c2cc(F)ccc2F)cc2nnn(Cc3cccc(C(F)(F)F)c3)c12]

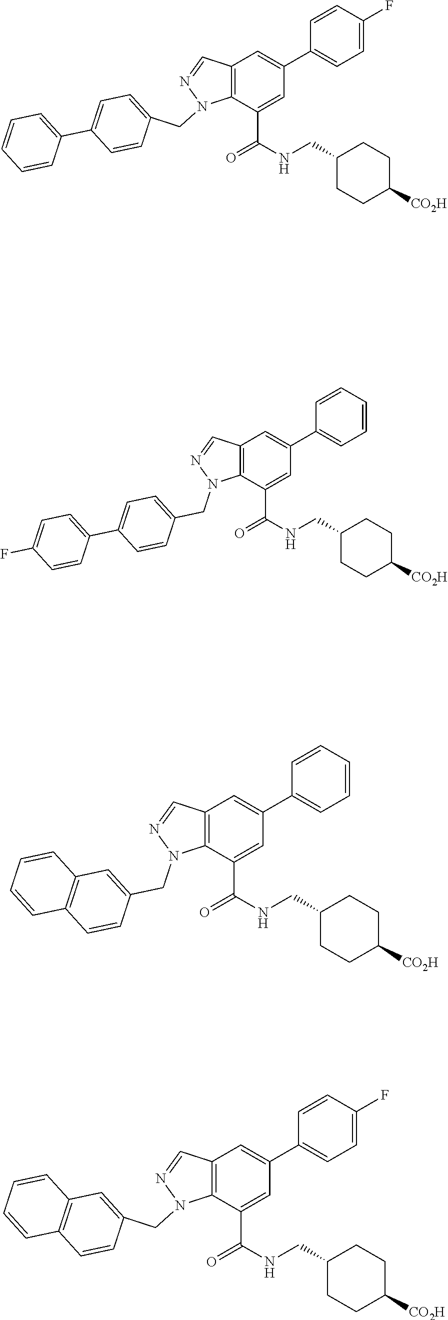 [SMILES: O=C(NC[C@H]1CC[C@H](C(=O)O)CC1)c1cc(-c2ccc(F)cc2)cc2cnn(Cc3ccc(-c4ccccc4)cc3)c12 ; O=C(NC[C@H]1CC[C@H](C(=O)O)CC1)c1cc(-c2ccccc2)cc2cnn(Cc3ccc(-c4ccc(F)cc4)cc3)c12 ; O=C(NC[C@H]1CC[C@H](C(=O)O)CC1)c1cc(-c2ccccc2)cc2cnn(Cc3ccc4ccccc4c3)c12 ; O=C(NC[C@H]1CC[C@H](C(=O)O)CC1)c1cc(-c2ccc(F)cc2)cc2cnn(Cc3ccc4ccccc4c3)c12]

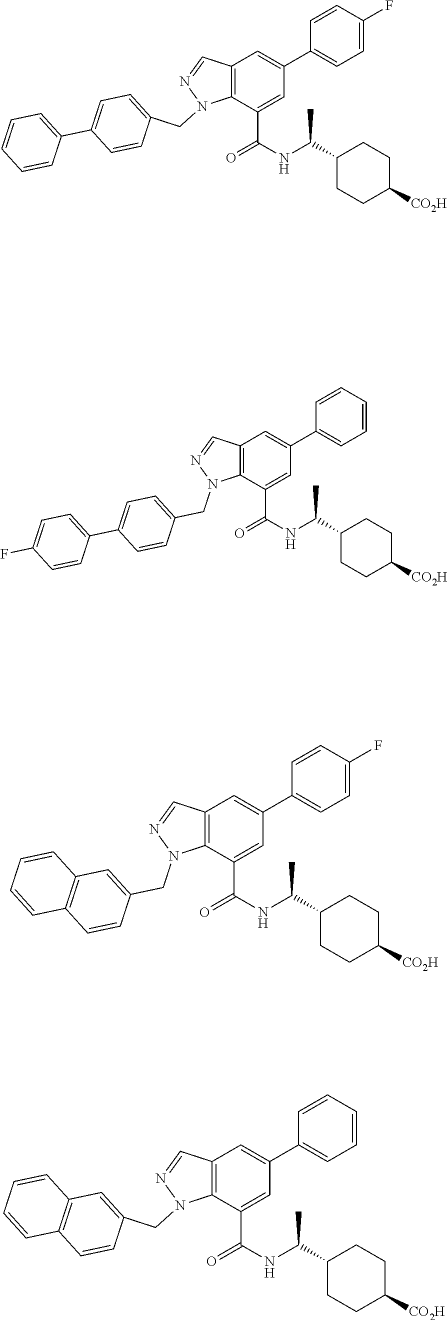 [SMILES: C[C@H](NC(=O)c1cc(-c2ccc(F)cc2)cc2cnn(Cc3ccc(-c4ccccc4)cc3)c12)[C@H]1CC[C@H](C(=O)O)CC1 ; C[C@H](NC(=O)c1cc(-c2ccccc2)cc2cnn(Cc3ccc(-c4ccc(F)cc4)cc3)c12)[C@H]1CC[C@H](C(=O)O)CC1 ; C[C@H](NC(=O)c1cc(-c2ccc(F)cc2)cc2cnn(Cc3ccc4ccccc4c3)c12)[C@H]1CC[C@H](C(=O)O)CC1 ; C[C@H](NC(=O)c1cc(-c2ccccc2)cc2cnn(Cc3ccc4ccccc4c3)c12)[C@H]1CC[C@H](C(=O)O)CC1]

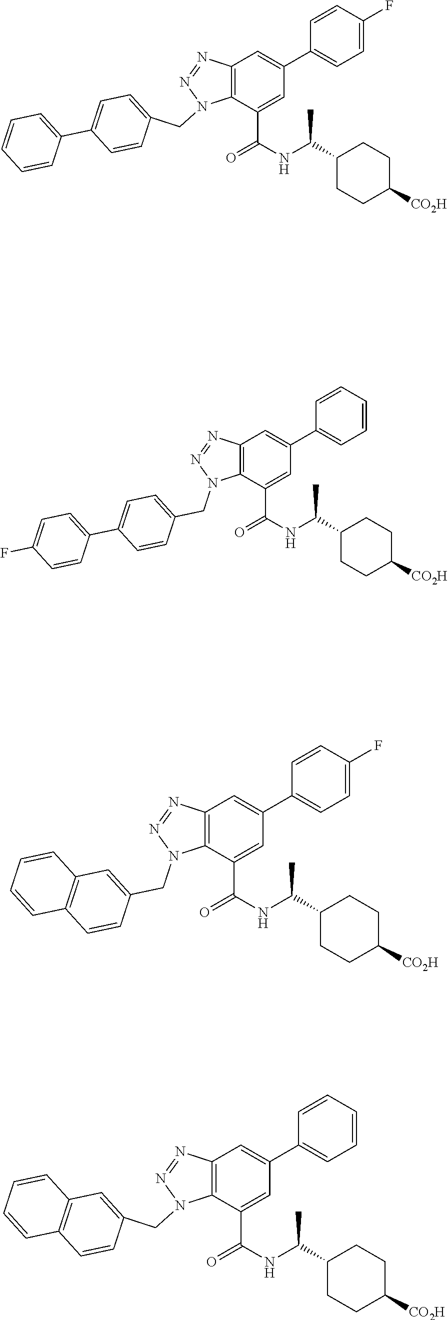 [SMILES: C[C@H](NC(=O)c1cc(-c2ccc(F)cc2)cc2nnn(Cc3ccc(-c4ccccc4)cc3)c12)[C@H]1CC[C@H](C(=O)O)CC1 ; C[C@H](NC(=O)c1cc(-c2ccccc2)cc2nnn(Cc3ccc(-c4ccc(F)cc4)cc3)c12)[C@H]1CC[C@H](C(=O)O)CC1 ; C[C@H](NC(=O)c1cc(-c2ccc(F)cc2)cc2nnn(Cc3ccc4ccccc4c3)c12)[C@H]1CC[C@H](C(=O)O)CC1 ; C[C@H](NC(=O)c1cc(-c2ccccc2)cc2nnn(Cc3ccc4ccccc4c3)c12)[C@H]1CC[C@H](C(=O)O)CC1]

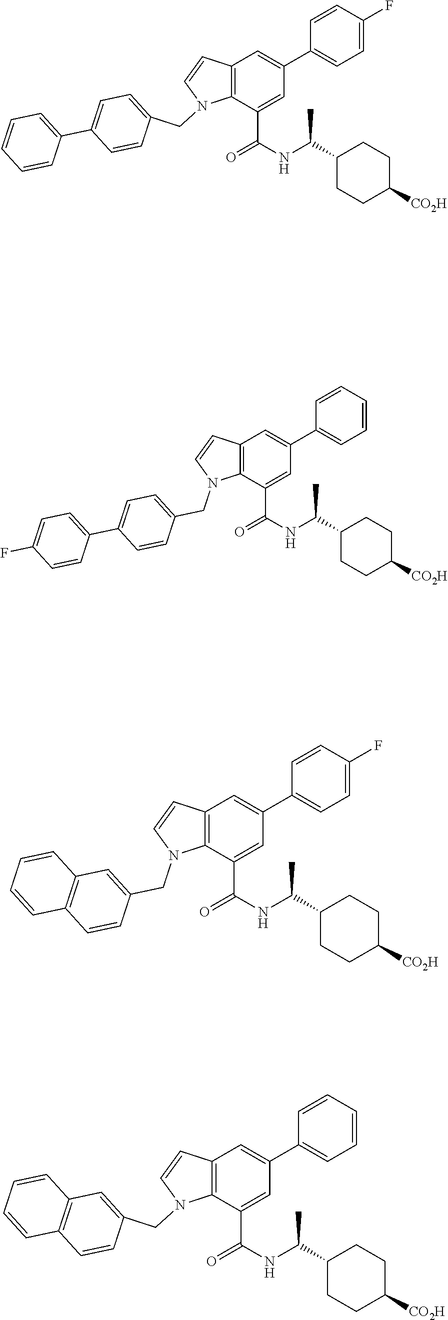 [SMILES: C[C@H](NC(=O)c1cc(-c2ccc(F)cc2)cc2ccn(Cc3ccc(-c4ccccc4)cc3)c12)[C@H]1CC[C@H](C(=O)O)CC1 ; C[C@H](NC(=O)c1cc(-c2ccccc2)cc2ccn(Cc3ccc(-c4ccc(F)cc4)cc3)c12)[C@H]1CC[C@H](C(=O)O)CC1 ; C[C@H](NC(=O)c1cc(-c2ccc(F)cc2)cc2ccn(Cc3ccc4ccccc4c3)c12)[C@H]1CC[C@H](C(=O)O)CC1 ; C[C@H](NC(=O)c1cc(-c2ccccc2)cc2ccn(Cc3ccc4ccccc4c3)c12)[C@H]1CC[C@H](C(=O)O)CC1]

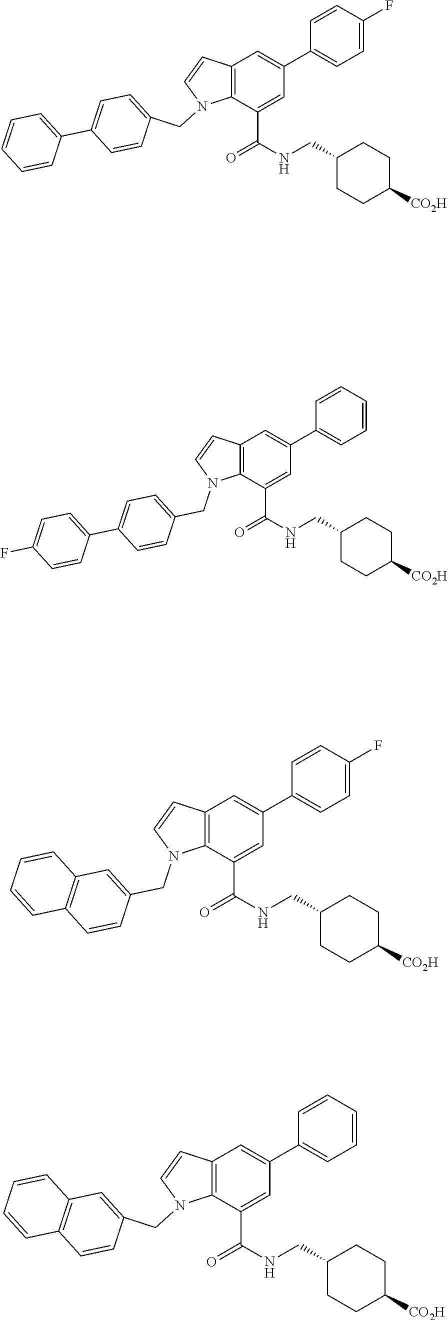 [SMILES: O=C(NC[C@H]1CC[C@H](C(=O)O)CC1)c1cc(-c2ccc(F)cc2)cc2ccn(Cc3ccc(-c4ccccc4)cc3)c12 ; O=C(NC[C@H]1CC[C@H](C(=O)O)CC1)c1cc(-c2ccccc2)cc2ccn(Cc3ccc(-c4ccc(F)cc4)cc3)c12 ; O=C(NC[C@H]1CC[C@H](C(=O)O)CC1)c1cc(-c2ccc(F)cc2)cc2ccn(Cc3ccc4ccccc4c3)c12 ; O=C(NC[C@H]1CC[C@H](C(=O)O)CC1)c1cc(-c2ccccc2)cc2ccn(Cc3ccc4ccccc4c3)c12]

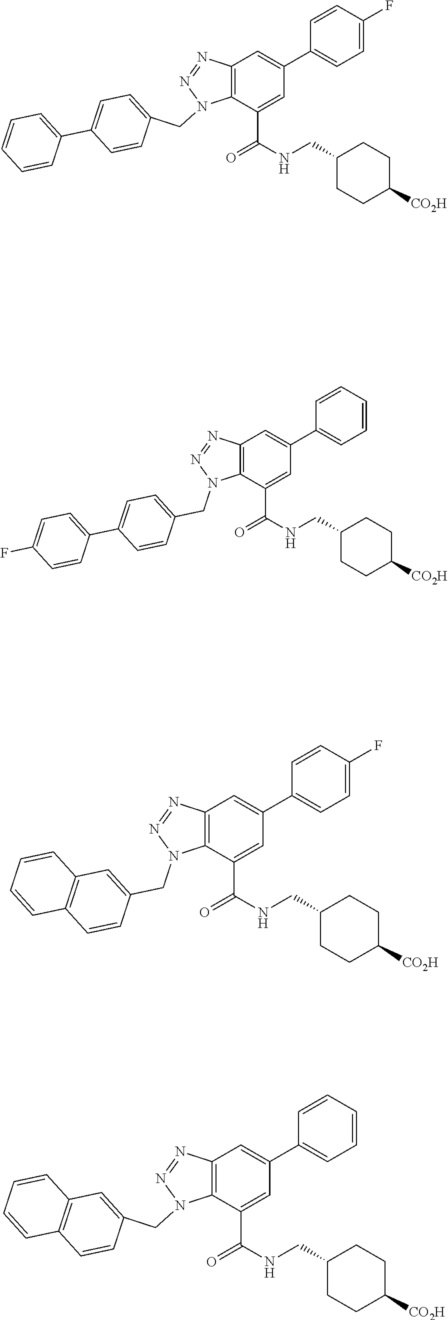 [SMILES: O=C(NC[C@H]1CC[C@H](C(=O)O)CC1)c1cc(-c2ccc(F)cc2)cc2nnn(Cc3ccc(-c4ccccc4)cc3)c12 ; O=C(NC[C@H]1CC[C@H](C(=O)O)CC1)c1cc(-c2ccccc2)cc2nnn(Cc3ccc(-c4ccc(F)cc4)cc3)c12 ; O=C(NC[C@H]1CC[C@H](C(=O)O)CC1)c1cc(-c2ccc(F)cc2)cc2nnn(Cc3ccc4ccccc4c3)c12 ; O=C(NC[C@H]1CC[C@H](C(=O)O)CC1)c1cc(-c2ccccc2)cc2nnn(Cc3ccc4ccccc4c3)c12]

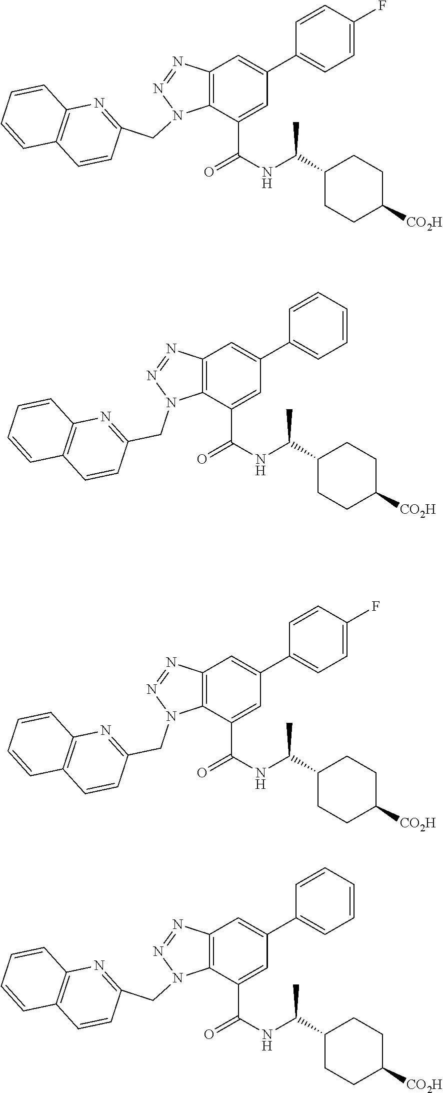 [SMILES: C[C@H](NC(=O)c1cc(-c2ccc(F)cc2)cc2nnn(Cc3ccc4ccccc4n3)c12)[C@H]1CC[C@H](C(=O)O)CC1 ; C[C@H](NC(=O)c1cc(-c2ccccc2)cc2nnn(Cc3ccc4ccccc4n3)c12)[C@H]1CC[C@H](C(=O)O)CC1 ; C[C@H](NC(=O)c1cc(-c2ccc(F)cc2)cc2nnn(Cc3ccc4ccccc4n3)c12)[C@H]1CC[C@H](C(=O)O)CC1 ; C[C@H](NC(=O)c1cc(-c2ccccc2)cc2nnn(Cc3ccc4ccccc4n3)c12)[C@H]1CC[C@H](C(=O)O)CC1]

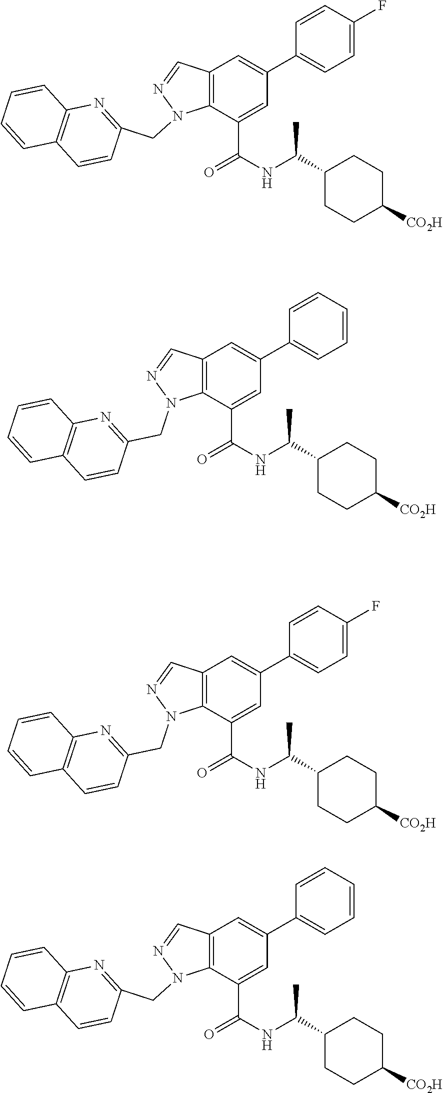 [SMILES: C[C@H](NC(=O)c1cc(-c2ccc(F)cc2)cc2cnn(Cc3ccc4ccccc4n3)c12)[C@H]1CC[C@H](C(=O)O)CC1 ; C[C@H](NC(=O)c1cc(-c2ccccc2)cc2cnn(Cc3ccc4ccccc4n3)c12)[C@H]1CC[C@H](C(=O)O)CC1 ; C[C@H](NC(=O)c1cc(-c2ccc(F)cc2)cc2cnn(Cc3ccc4ccccc4n3)c12)[C@H]1CC[C@H](C(=O)O)CC1 ; C[C@H](NC(=O)c1cc(-c2ccccc2)cc2cnn(Cc3ccc4ccccc4n3)c12)[C@H]1CC[C@H](C(=O)O)CC1]

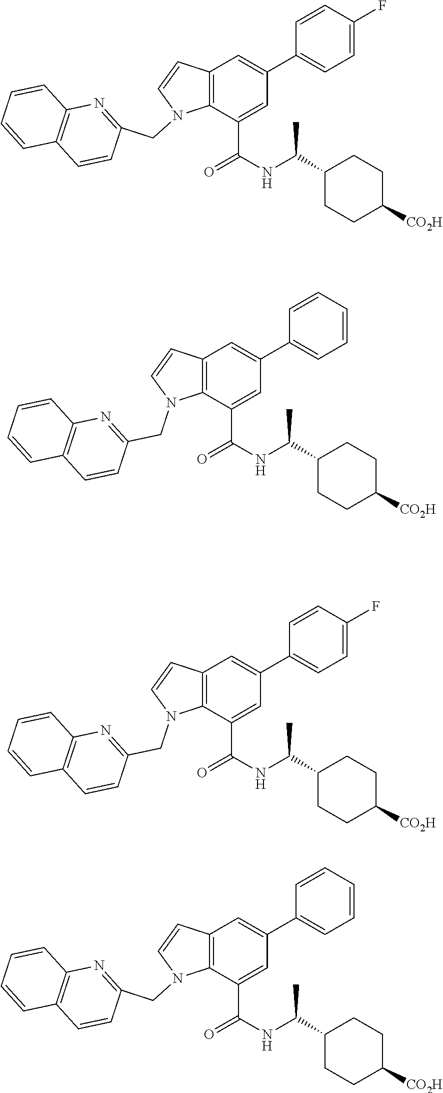 [SMILES: C[C@H](NC(=O)c1cc(-c2ccc(F)cc2)cc2ccn(Cc3ccc4ccccc4n3)c12)[C@H]1CC[C@H](C(=O)O)CC1 ; C[C@H](NC(=O)c1cc(-c2ccccc2)cc2ccn(Cc3ccc4ccccc4n3)c12)[C@H]1CC[C@H](C(=O)O)CC1 ; C[C@H](NC(=O)c1cc(-c2ccc(F)cc2)cc2ccn(Cc3ccc4ccccc4n3)c12)[C@H]1CC[C@H](C(=O)O)CC1 ; C[C@H](NC(=O)c1cc(-c2ccccc2)cc2ccn(Cc3ccc4ccccc4n3)c12)[C@H]1CC[C@H](C(=O)O)CC1]

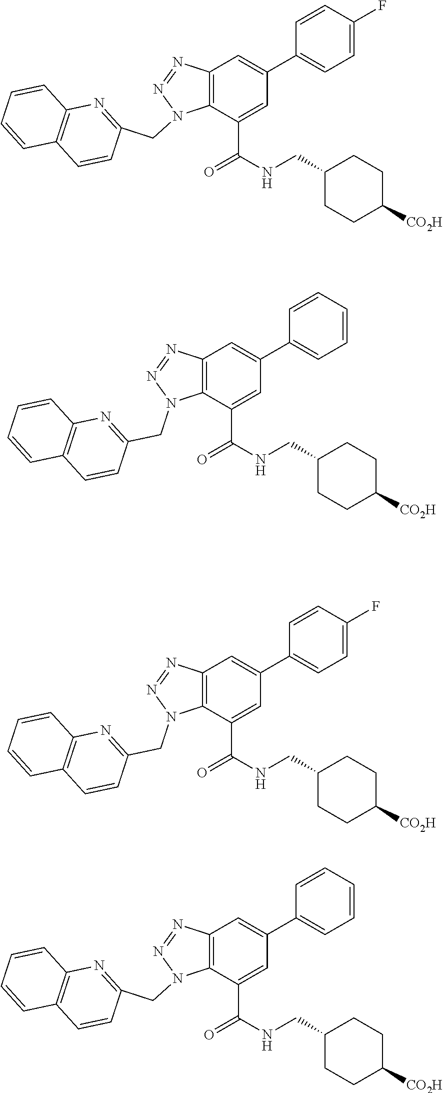 [SMILES: O=C(NC[C@H]1CC[C@H](C(=O)O)CC1)c1cc(-c2ccc(F)cc2)cc2nnn(Cc3ccc4ccccc4n3)c12 ; O=C(NC[C@H]1CC[C@H](C(=O)O)CC1)c1cc(-c2ccccc2)cc2nnn(Cc3ccc4ccccc4n3)c12 ; O=C(NC[C@H]1CC[C@H](C(=O)O)CC1)c1cc(-c2ccc(F)cc2)cc2nnn(Cc3ccc4ccccc4n3)c12 ; O=C(NC[C@H]1CC[C@H](C(=O)O)CC1)c1cc(-c2ccccc2)cc2nnn(Cc3ccc4ccccc4n3)c12]

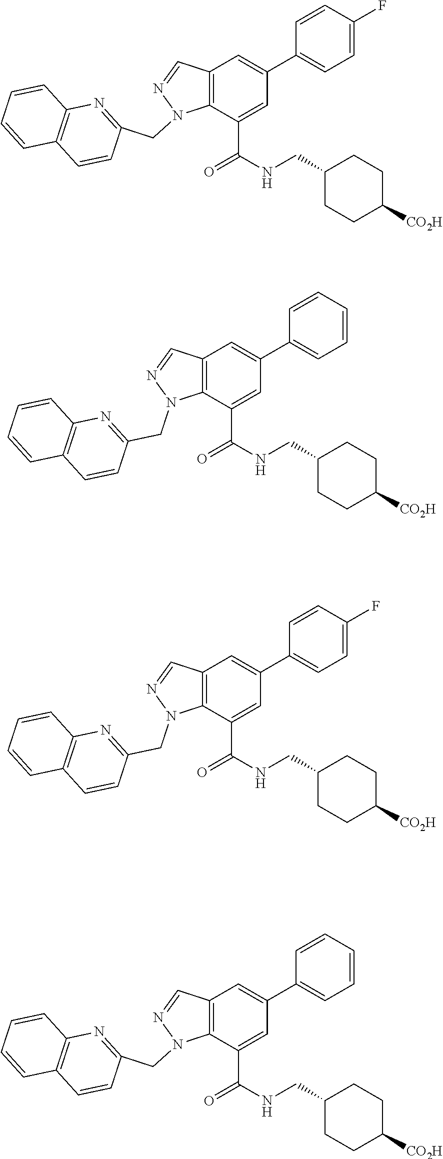 [SMILES: O=C(NC[C@H]1CC[C@H](C(=O)O)CC1)c1cc(-c2ccc(F)cc2)cc2cnn(Cc3ccc4ccccc4n3)c12 ; O=C(NC[C@H]1CC[C@H](C(=O)O)CC1)c1cc(-c2ccccc2)cc2cnn(Cc3ccc4ccccc4n3)c12 ; O=C(NC[C@H]1CC[C@H](C(=O)O)CC1)c1cc(-c2ccc(F)cc2)cc2cnn(Cc3ccc4ccccc4n3)c12 ; O=C(NC[C@H]1CC[C@H](C(=O)O)CC1)c1cc(-c2ccccc2)cc2cnn(Cc3ccc4ccccc4n3)c12]

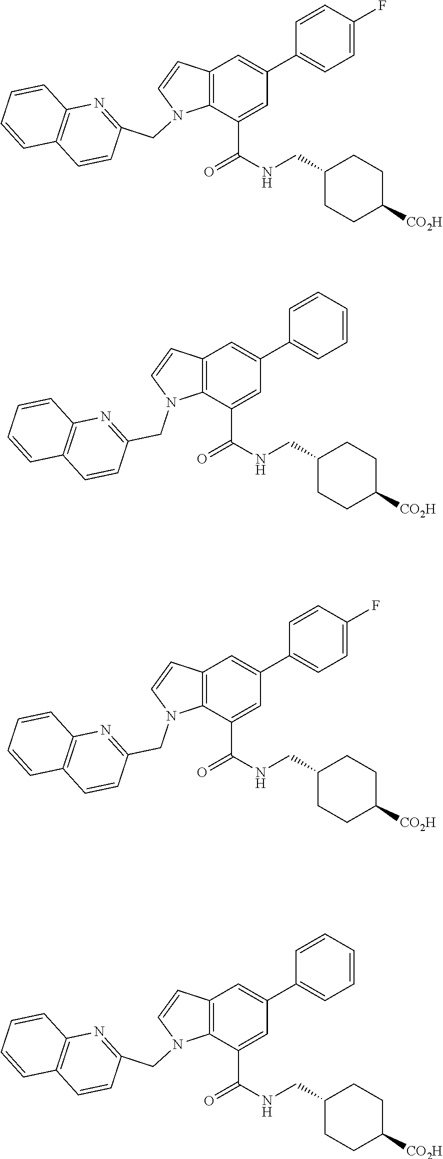 [SMILES: O=C(NC[C@H]1CC[C@H](C(=O)O)CC1)c1cc(-c2ccc(F)cc2)cc2ccn(Cc3ccc4ccccc4n3)c12 ; O=C(NC[C@H]1CC[C@H](C(=O)O)CC1)c1cc(-c2ccccc2)cc2ccn(Cc3ccc4ccccc4n3)c12 ; O=C(NC[C@H]1CC[C@H](C(=O)O)CC1)c1cc(-c2ccc(F)cc2)cc2ccn(Cc3ccc4ccccc4n3)c12 ; O=C(NC[C@H]1CC[C@H](C(=O)O)CC1)c1cc(-c2ccccc2)cc2ccn(Cc3ccc4ccccc4n3)c12]

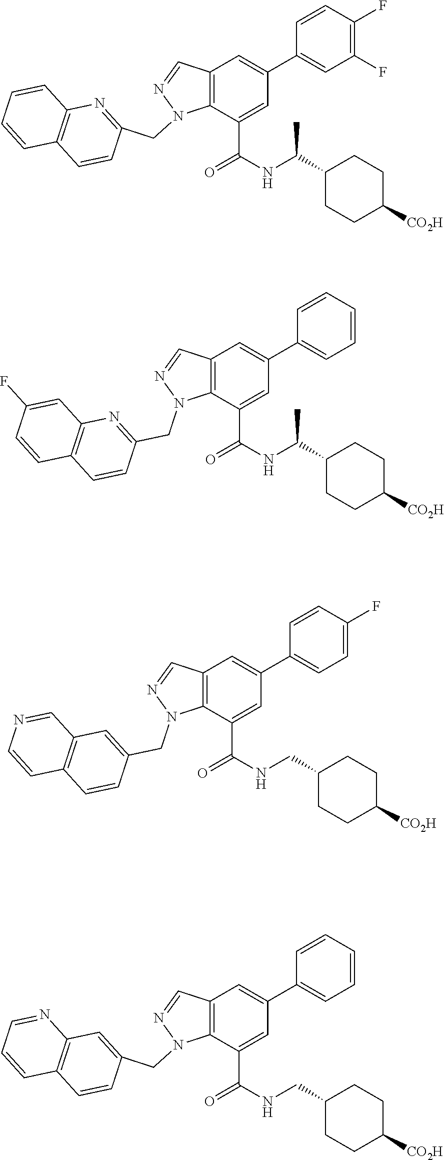 [SMILES: C[C@H](NC(=O)c1cc(-c2ccc(F)c(F)c2)cc2cnn(Cc3ccc4ccccc4n3)c12)[C@H]1CC[C@H](C(=O)O)CC1 ; C[C@H](NC(=O)c1cc(-c2ccccc2)cc2cnn(Cc3ccc4ccc(F)cc4n3)c12)[C@H]1CC[C@H](C(=O)O)CC1 ; O=C(NC[C@H]1CC[C@H](C(=O)O)CC1)c1cc(-c2ccc(F)cc2)cc2cnn(Cc3ccc4ccncc4c3)c12 ; O=C(NC[C@H]1CC[C@H](C(=O)O)CC1)c1cc(-c2ccccc2)cc2cnn(Cc3ccc4cccnc4c3)c12]

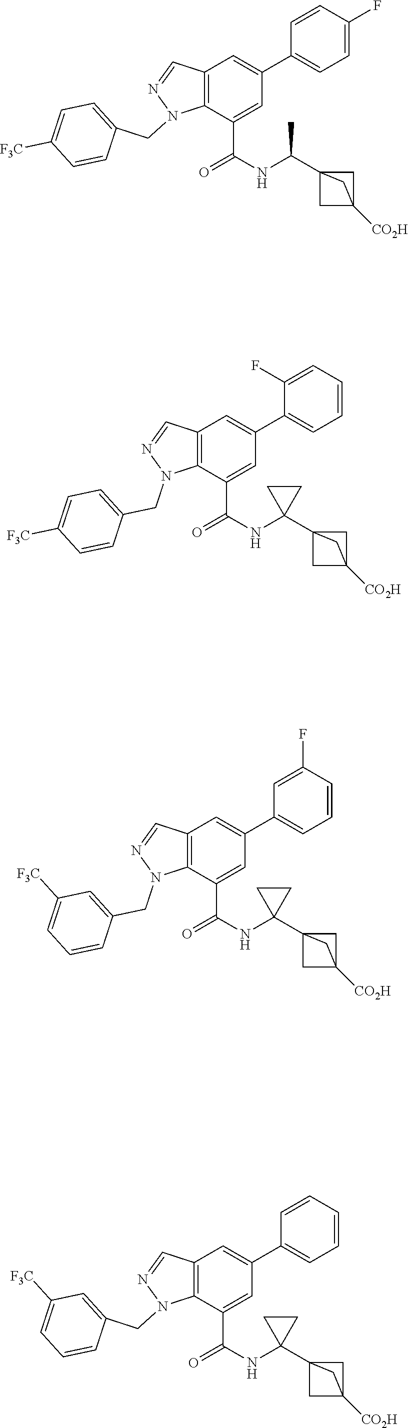 [SMILES: C[C@H](NC(=O)c1cc(-c2ccc(F)cc2)cc2cnn(Cc3ccc(C(F)(F)F)cc3)c12)C12CC(C(=O)O)(C1)C2 ; O=C(NC1(C23CC(C(=O)O)(C2)C3)CC1)c1cc(-c2ccccc2F)cc2cnn(Cc3ccc(C(F)(F)F)cc3)c12 ; O=C(NC1(C23CC(C(=O)O)(C2)C3)CC1)c1cc(-c2cccc(F)c2)cc2cnn(Cc3cccc(C(F)(F)F)c3)c12 ; O=C(NC1(C23CC(C(=O)O)(C2)C3)CC1)c1cc(-c2ccccc2)cc2cnn(Cc3cccc(C(F)(F)F)c3)c12]

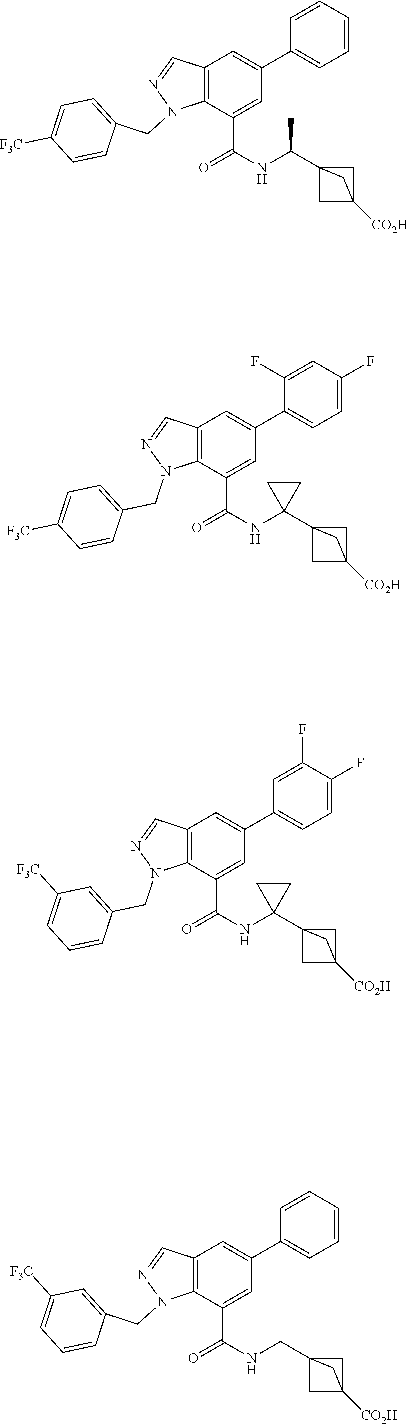 [SMILES: C[C@H](NC(=O)c1cc(-c2ccccc2)cc2cnn(Cc3ccc(C(F)(F)F)cc3)c12)C12CC(C(=O)O)(C1)C2 ; O=C(NC1(C23CC(C(=O)O)(C2)C3)CC1)c1cc(-c2ccc(F)cc2F)cc2cnn(Cc3ccc(C(F)(F)F)cc3)c12 ; O=C(NC1(C23CC(C(=O)O)(C2)C3)CC1)c1cc(-c2ccc(F)c(F)c2)cc2cnn(Cc3cccc(C(F)(F)F)c3)c12 ; O=C(NCC12CC(C(=O)O)(C1)C2)c1cc(-c2ccccc2)cc2cnn(Cc3cccc(C(F)(F)F)c3)c12]

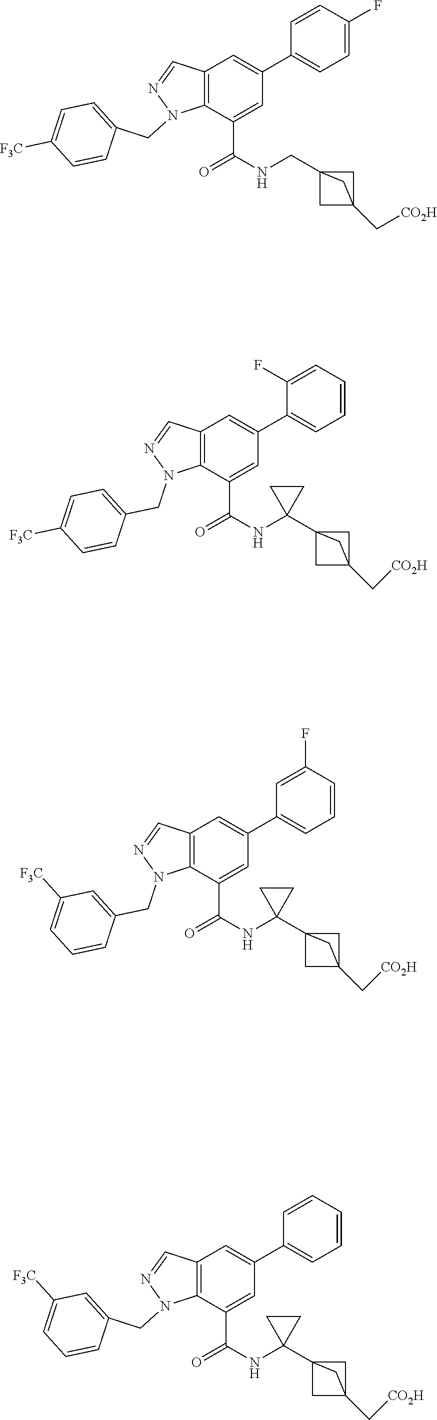 [SMILES: O=C(O)CC12CC(CNC(=O)c3cc(-c4ccc(F)cc4)cc4cnn(Cc5ccc(C(F)(F)F)cc5)c34)(C1)C2 ; O=C(O)CC12CC(C3(NC(=O)c4cc(-c5ccccc5F)cc5cnn(Cc6ccc(C(F)(F)F)cc6)c45)CC3)(C1)C2 ; O=C(O)CC12CC(C3(NC(=O)c4cc(-c5cccc(F)c5)cc5cnn(Cc6cccc(C(F)(F)F)c6)c45)CC3)(C1)C2 ; O=C(O)CC12CC(C3(NC(=O)c4cc(-c5ccccc5)cc5cnn(Cc6cccc(C(F)(F)F)c6)c45)CC3)(C1)C2]

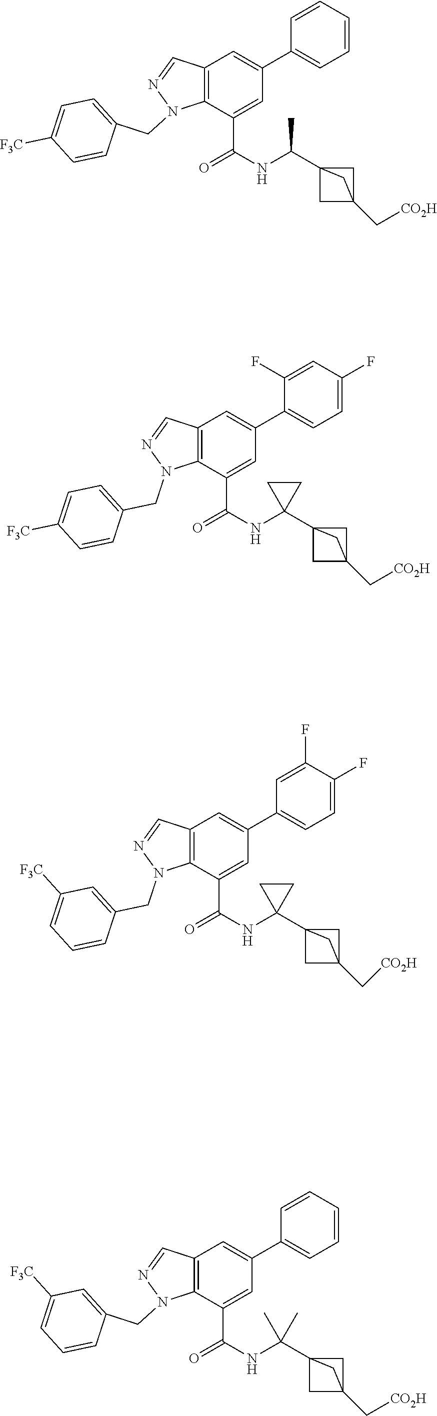 [SMILES: C[C@H](NC(=O)c1cc(-c2ccccc2)cc2cnn(Cc3ccc(C(F)(F)F)cc3)c12)C12CC(CC(=O)O)(C1)C2 ; O=C(O)CC12CC(C3(NC(=O)c4cc(-c5ccc(F)cc5F)cc5cnn(Cc6ccc(C(F)(F)F)cc6)c45)CC3)(C1)C2 ; O=C(O)CC12CC(C3(NC(=O)c4cc(-c5ccc(F)c(F)c5)cc5cnn(Cc6cccc(C(F)(F)F)c6)c45)CC3)(C1)C2 ; CC(C)(NC(=O)c1cc(-c2ccccc2)cc2cnn(Cc3cccc(C(F)(F)F)c3)c12)C12CC(CC(=O)O)(C1)C2]

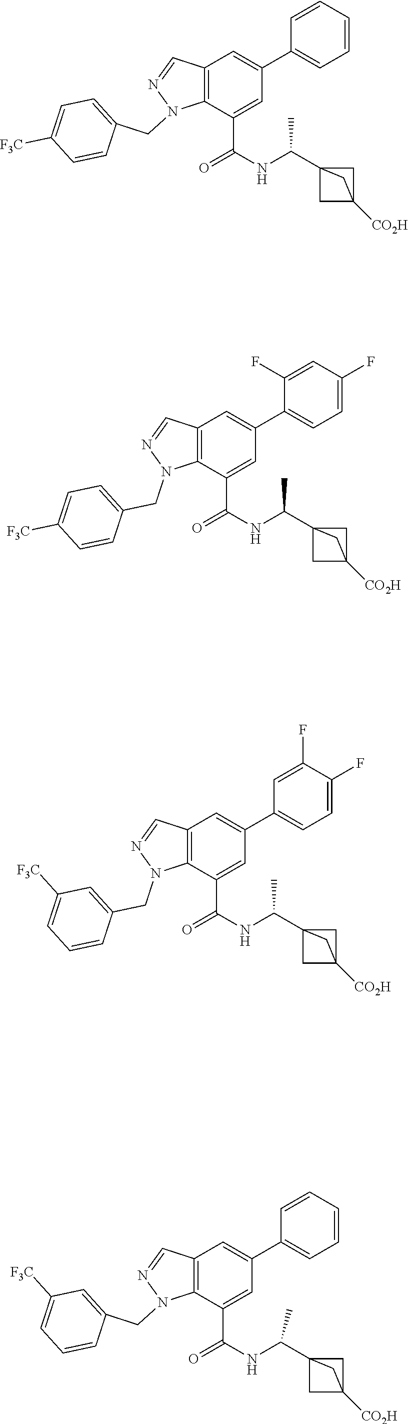 [SMILES: C[C@@H](NC(=O)c1cc(-c2ccccc2)cc2cnn(Cc3ccc(C(F)(F)F)cc3)c12)C12CC(C(=O)O)(C1)C2 ; C[C@H](NC(=O)c1cc(-c2ccc(F)cc2F)cc2cnn(Cc3ccc(C(F)(F)F)cc3)c12)C12CC(C(=O)O)(C1)C2 ; C[C@@H](NC(=O)c1cc(-c2ccc(F)c(F)c2)cc2cnn(Cc3cccc(C(F)(F)F)c3)c12)C12CC(C(=O)O)(C1)C2 ; C[C@@H](NC(=O)c1cc(-c2ccccc2)cc2cnn(Cc3cccc(C(F)(F)F)c3)c12)C12CC(C(=O)O)(C1)C2]

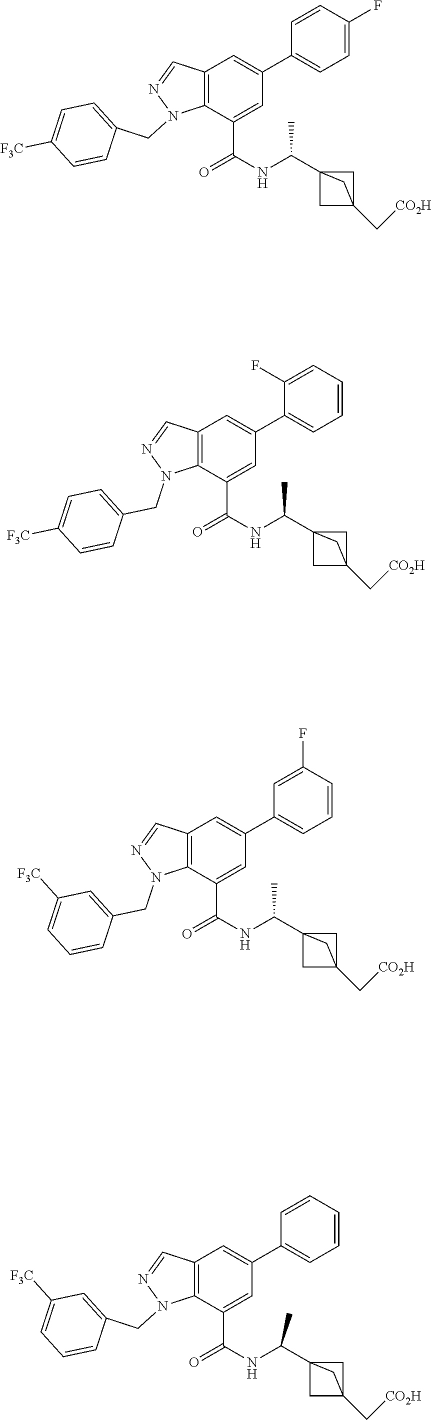 [SMILES: C[C@@H](NC(=O)c1cc(-c2ccc(F)cc2)cc2cnn(Cc3ccc(C(F)(F)F)cc3)c12)C12CC(CC(=O)O)(C1)C2 ; C[C@H](NC(=O)c1cc(-c2ccccc2F)cc2cnn(Cc3ccc(C(F)(F)F)cc3)c12)C12CC(CC(=O)O)(C1)C2 ; C[C@@H](NC(=O)c1cc(-c2cccc(F)c2)cc2cnn(Cc3cccc(C(F)(F)F)c3)c12)C12CC(CC(=O)O)(C1)C2 ; C[C@H](NC(=O)c1cc(-c2ccccc2)cc2cnn(Cc3cccc(C(F)(F)F)c3)c12)C12CC(CC(=O)O)(C1)C2]

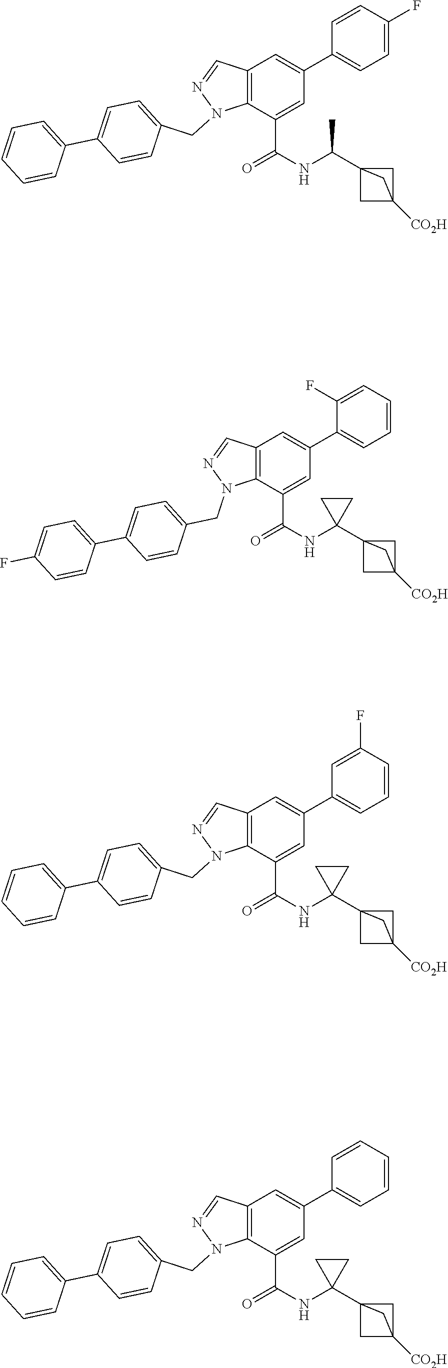 [SMILES: C[C@H](NC(=O)c1cc(-c2ccc(F)cc2)cc2cnn(Cc3ccc(-c4ccccc4)cc3)c12)C12CC(C(=O)O)(C1)C2 ; O=C(NC1(C23CC(C(=O)O)(C2)C3)CC1)c1cc(-c2ccccc2F)cc2cnn(Cc3ccc(-c4ccc(F)cc4)cc3)c12 ; O=C(NC1(C23CC(C(=O)O)(C2)C3)CC1)c1cc(-c2cccc(F)c2)cc2cnn(Cc3ccc(-c4ccccc4)cc3)c12 ; O=C(NC1(C23CC(C(=O)O)(C2)C3)CC1)c1cc(-c2ccccc2)cc2cnn(Cc3ccc(-c4ccccc4)cc3)c12]

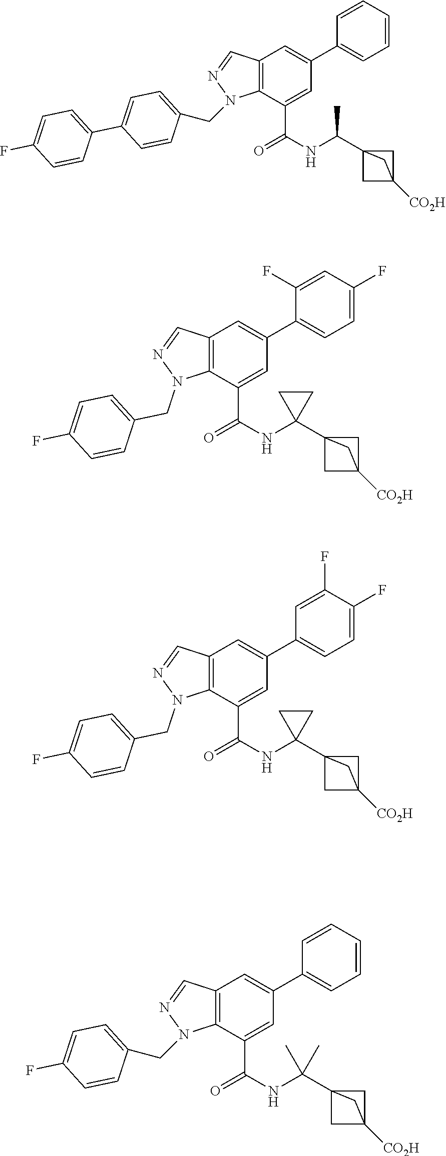 [SMILES: C[C@H](NC(=O)c1cc(-c2ccccc2)cc2cnn(Cc3ccc(-c4ccc(F)cc4)cc3)c12)C12CC(C(=O)O)(C1)C2 ; O=C(NC1(C23CC(C(=O)O)(C2)C3)CC1)c1cc(-c2ccc(F)cc2F)cc2cnn(Cc3ccc(F)cc3)c12 ; O=C(NC1(C23CC(C(=O)O)(C2)C3)CC1)c1cc(-c2ccc(F)c(F)c2)cc2cnn(Cc3ccc(F)cc3)c12 ; CC(C)(NC(=O)c1cc(-c2ccccc2)cc2cnn(Cc3ccc(F)cc3)c12)C12CC(C(=O)O)(C1)C2]

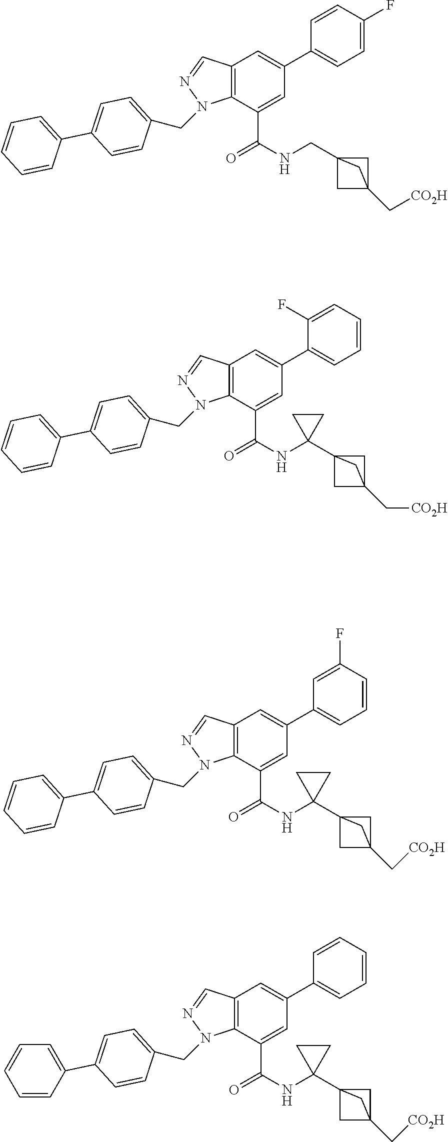 [SMILES: O=C(O)CC12CC(CNC(=O)c3cc(-c4ccc(F)cc4)cc4cnn(Cc5ccc(-c6ccccc6)cc5)c34)(C1)C2 ; O=C(O)CC12CC(C3(NC(=O)c4cc(-c5ccccc5F)cc5cnn(Cc6ccc(-c7ccccc7)cc6)c45)CC3)(C1)C2 ; O=C(O)CC12CC(C3(NC(=O)c4cc(-c5cccc(F)c5)cc5cnn(Cc6ccc(-c7ccccc7)cc6)c45)CC3)(C1)C2 ; O=C(O)CC12CC(C3(NC(=O)c4cc(-c5ccccc5)cc5cnn(Cc6ccc(-c7ccccc7)cc6)c45)CC3)(C1)C2]

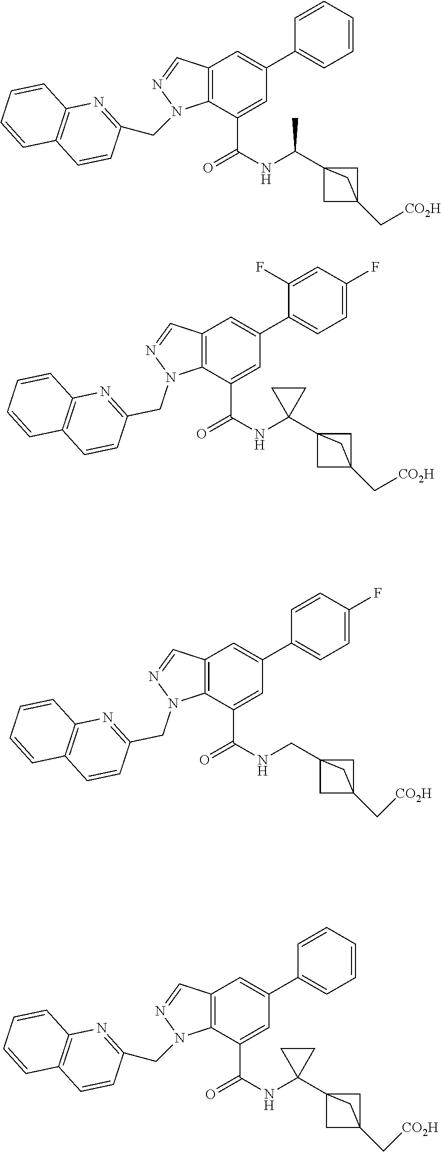

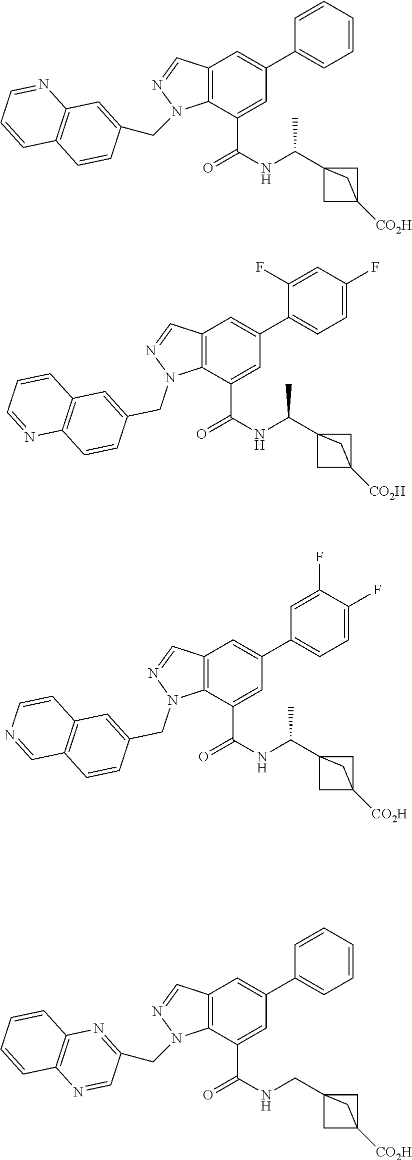

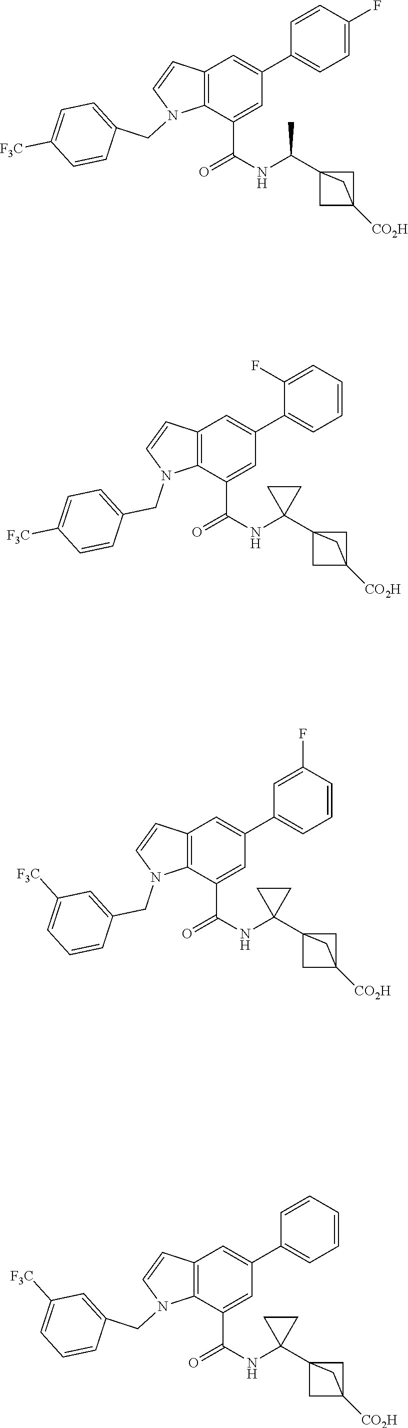 [SMILES: C[C@H](NC(=O)c1cc(-c2ccc(F)cc2)cc2ccn(Cc3ccc(C(F)(F)F)cc3)c12)C12CC(C(=O)O)(C1)C2 ; O=C(NC1(C23CC(C(=O)O)(C2)C3)CC1)c1cc(-c2ccccc2F)cc2ccn(Cc3ccc(C(F)(F)F)cc3)c12 ; O=C(NC1(C23CC(C(=O)O)(C2)C3)CC1)c1cc(-c2cccc(F)c2)cc2ccn(Cc3cccc(C(F)(F)F)c3)c12 ; O=C(NC1(C23CC(C(=O)O)(C2)C3)CC1)c1cc(-c2ccccc2)cc2ccn(Cc3cccc(C(F)(F)F)c3)c12]

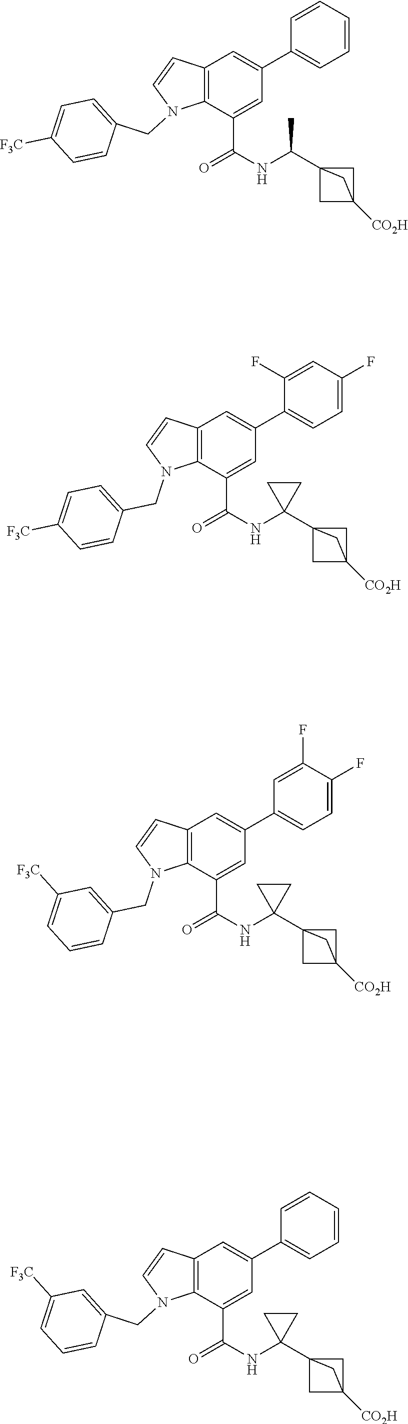 [SMILES: C[C@H](NC(=O)c1cc(-c2ccccc2)cc2ccn(Cc3ccc(C(F)(F)F)cc3)c12)C12CC(C(=O)O)(C1)C2 ; O=C(NC1(C23CC(C(=O)O)(C2)C3)CC1)c1cc(-c2ccc(F)cc2F)cc2ccn(Cc3ccc(C(F)(F)F)cc3)c12 ; O=C(NC1(C23CC(C(=O)O)(C2)C3)CC1)c1cc(-c2ccc(F)c(F)c2)cc2ccn(Cc3cccc(C(F)(F)F)c3)c12 ; O=C(NC1(C23CC(C(=O)O)(C2)C3)CC1)c1cc(-c2ccccc2)cc2ccn(Cc3cccc(C(F)(F)F)c3)c12]

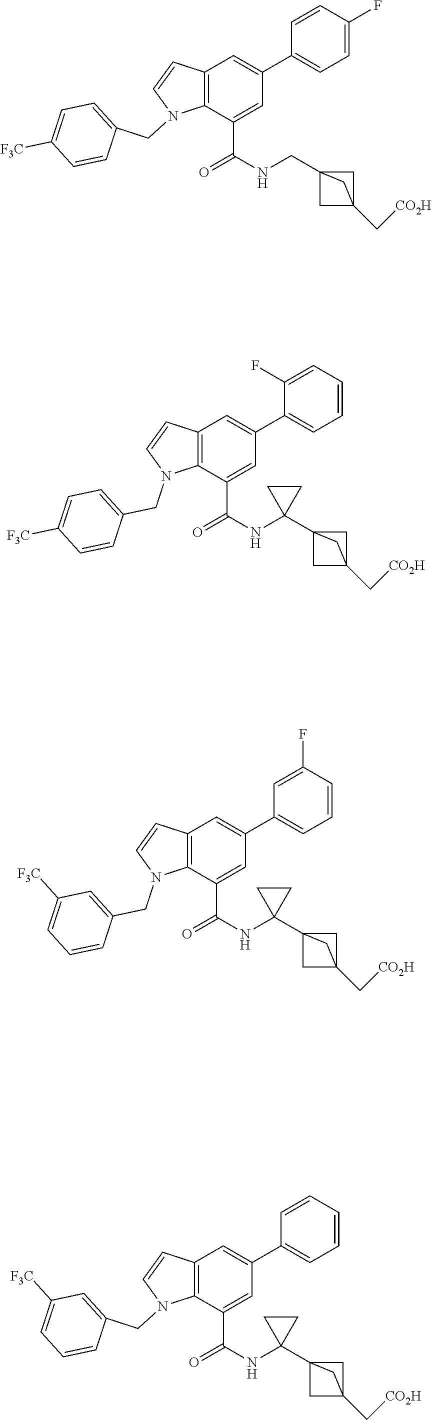 [SMILES: O=C(O)CC12CC(CNC(=O)c3cc(-c4ccc(F)cc4)cc4ccn(Cc5ccc(C(F)(F)F)cc5)c34)(C1)C2 ; O=C(O)CC12CC(C3(NC(=O)c4cc(-c5ccccc5F)cc5ccn(Cc6ccc(C(F)(F)F)cc6)c45)CC3)(C1)C2 ; O=C(O)CC12CC(C3(NC(=O)c4cc(-c5cccc(F)c5)cc5ccn(Cc6cccc(C(F)(F)F)c6)c45)CC3)(C1)C2 ; O=C(O)CC12CC(C3(NC(=O)c4cc(-c5ccccc5)cc5ccn(Cc6cccc(C(F)(F)F)c6)c45)CC3)(C1)C2]

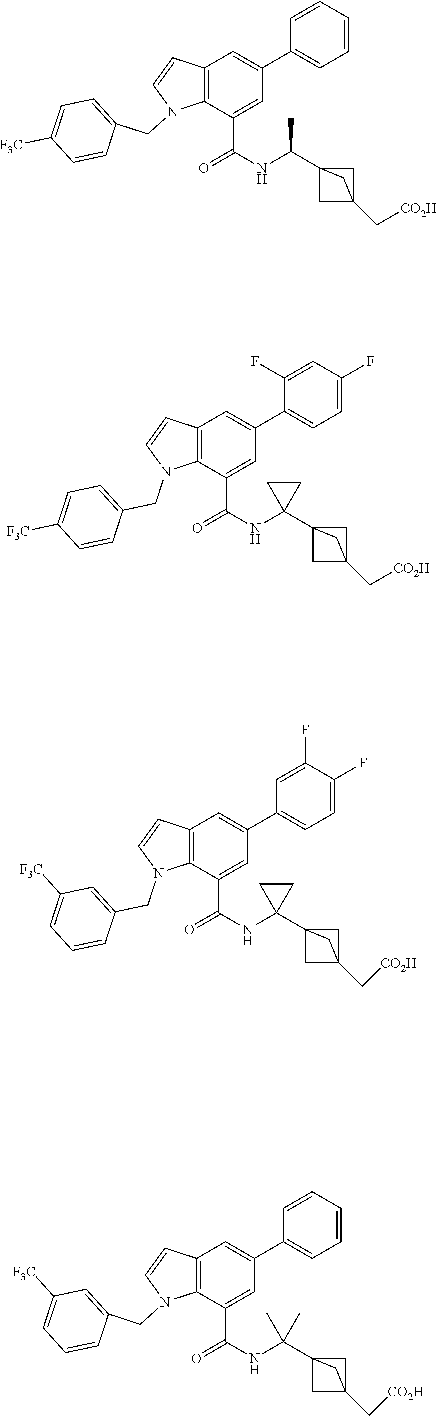 [SMILES: C[C@H](NC(=O)c1cc(-c2ccccc2)cc2ccn(Cc3ccc(C(F)(F)F)cc3)c12)C12CC(CC(=O)O)(C1)C2 ; O=C(O)CC12CC(C3(NC(=O)c4cc(-c5ccc(F)cc5F)cc5ccn(Cc6ccc(C(F)(F)F)cc6)c45)CC3)(C1)C2 ; O=C(O)CC12CC(C3(NC(=O)c4cc(-c5ccc(F)c(F)c5)cc5ccn(Cc6cccc(C(F)(F)F)c6)c45)CC3)(C1)C2 ; CC(C)(NC(=O)c1cc(-c2ccccc2)cc2ccn(Cc3cccc(C(F)(F)F)c3)c12)C12CC(CC(=O)O)(C1)C2]

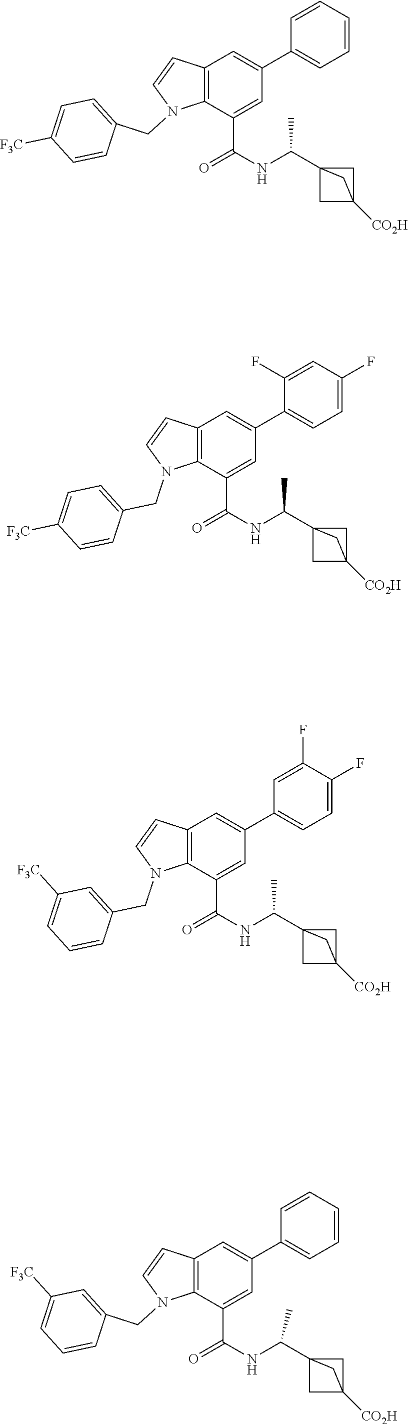 [SMILES: C[C@@H](NC(=O)c1cc(-c2ccccc2)cc2ccn(Cc3ccc(C(F)(F)F)cc3)c12)C12CC(C(=O)O)(C1)C2 ; C[C@H](NC(=O)c1cc(-c2ccc(F)cc2F)cc2ccn(Cc3ccc(C(F)(F)F)cc3)c12)C12CC(C(=O)O)(C1)C2 ; C[C@@H](NC(=O)c1cc(-c2ccc(F)c(F)c2)cc2ccn(Cc3cccc(C(F)(F)F)c3)c12)C12CC(C(=O)O)(C1)C2 ; C[C@@H](NC(=O)c1cc(-c2ccccc2)cc2ccn(Cc3cccc(C(F)(F)F)c3)c12)C12CC(C(=O)O)(C1)C2]

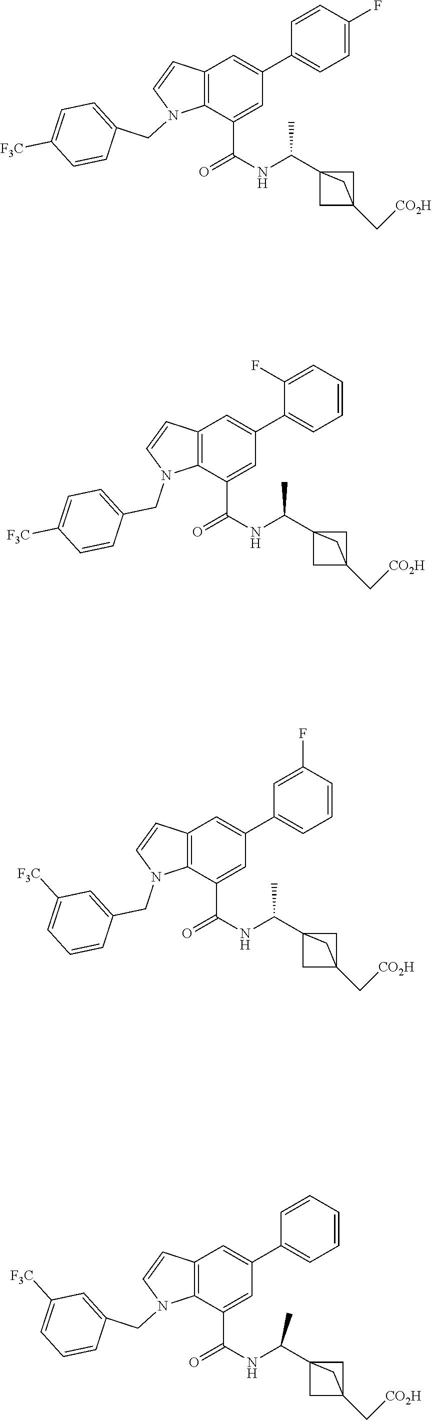 [SMILES: C[C@@H](NC(=O)c1cc(-c2ccc(F)cc2)cc2ccn(Cc3ccc(C(F)(F)F)cc3)c12)C12CC(CC(=O)O)(C1)C2 ; C[C@H](NC(=O)c1cc(-c2ccccc2F)cc2ccn(Cc3ccc(C(F)(F)F)cc3)c12)C12CC(CC(=O)O)(C1)C2 ; C[C@@H](NC(=O)c1cc(-c2cccc(F)c2)cc2ccn(Cc3cccc(C(F)(F)F)c3)c12)C12CC(CC(=O)O)(C1)C2 ; C[C@H](NC(=O)c1cc(-c2ccccc2)cc2ccn(Cc3cccc(C(F)(F)F)c3)c12)C12CC(CC(=O)O)(C1)C2]

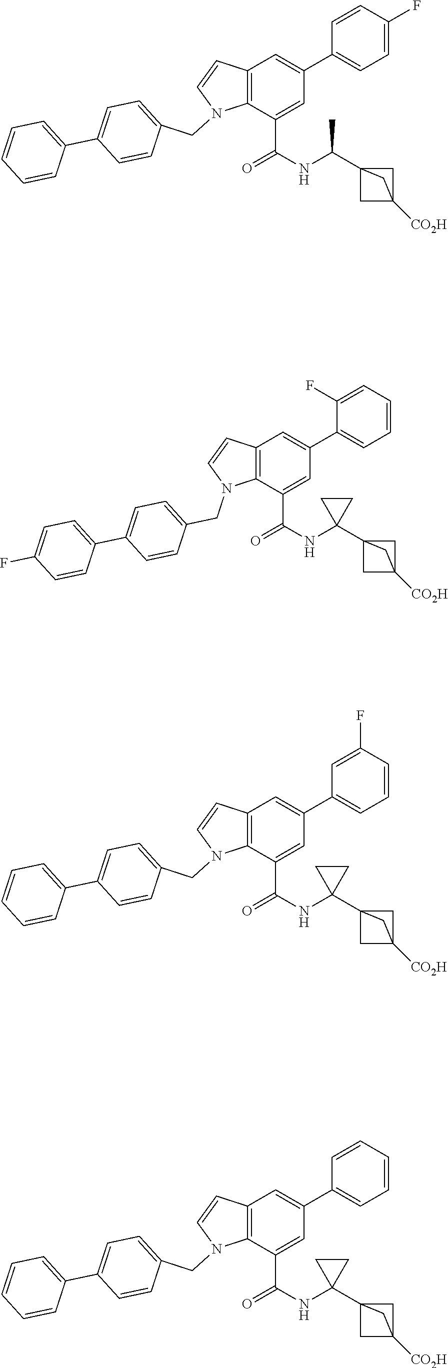 [SMILES: C[C@H](NC(=O)c1cc(-c2ccc(F)cc2)cc2ccn(Cc3ccc(-c4ccccc4)cc3)c12)C12CC(C(=O)O)(C1)C2 ; O=C(NC1(C23CC(C(=O)O)(C2)C3)CC1)c1cc(-c2ccccc2F)cc2ccn(Cc3ccc(-c4ccc(F)cc4)cc3)c12 ; O=C(NC1(C23CC(C(=O)O)(C2)C3)CC1)c1cc(-c2cccc(F)c2)cc2ccn(Cc3ccc(-c4ccccc4)cc3)c12 ; O=C(NC1(C23CC(C(=O)O)(C2)C3)CC1)c1cc(-c2ccccc2)cc2ccn(Cc3ccc(-c4ccccc4)cc3)c12]

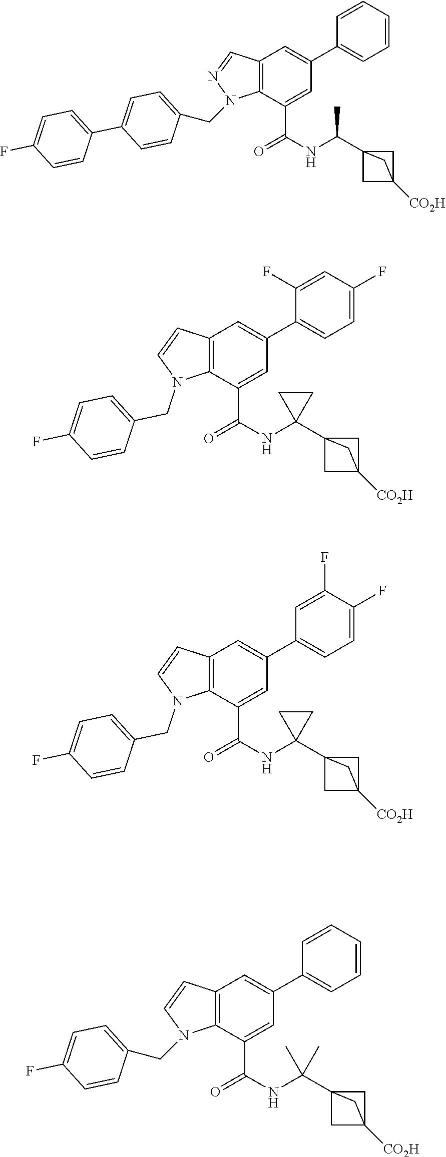 [SMILES: C[C@H](NC(=O)c1cc(-c2ccccc2)cc2cnn(Cc3ccc(-c4ccc(F)cc4)cc3)c12)C12CC(C(=O)O)(C1)C2 ; O=C(NC1(C23CC(C(=O)O)(C2)C3)CC1)c1cc(-c2ccc(F)cc2F)cc2ccn(Cc3ccc(F)cc3)c12 ; O=C(NC1(C23CC(C(=O)O)(C2)C3)CC1)c1cc(-c2ccc(F)c(F)c2)cc2ccn(Cc3ccc(F)cc3)c12 ; CC(C)(NC(=O)c1cc(-c2ccccc2)cc2ccn(Cc3ccc(F)cc3)c12)C12CC(C(=O)O)(C1)C2]

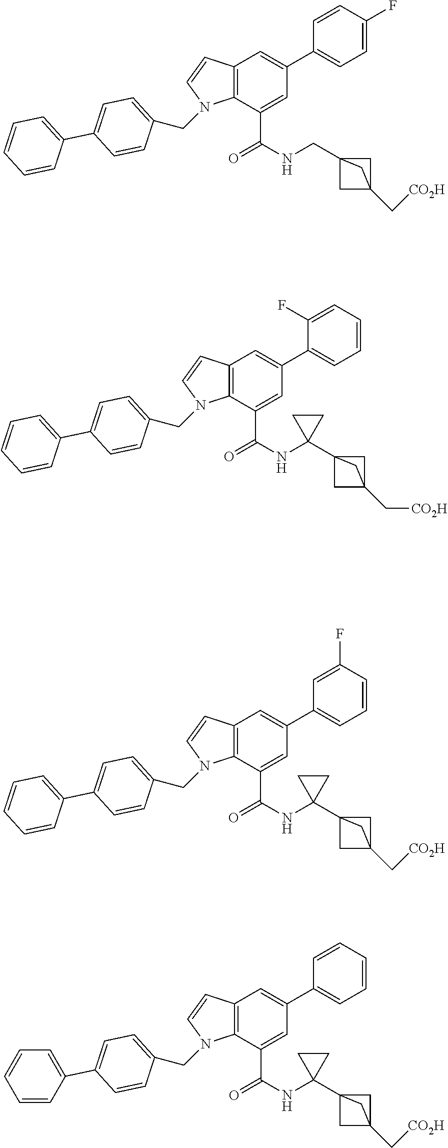 [SMILES: O=C(O)CC12CC(CNC(=O)c3cc(-c4ccc(F)cc4)cc4ccn(Cc5ccc(-c6ccccc6)cc5)c34)(C1)C2 ; O=C(O)CC12CC(C3(NC(=O)c4cc(-c5ccccc5F)cc5ccn(Cc6ccc(-c7ccccc7)cc6)c45)CC3)(C1)C2 ; O=C(O)CC12CC(C3(NC(=O)c4cc(-c5cccc(F)c5)cc5ccn(Cc6ccc(-c7ccccc7)cc6)c45)CC3)(C1)C2 ; O=C(O)CC12CC(C3(NC(=O)c4cc(-c5ccccc5)cc5ccn(Cc6ccc(-c7ccccc7)cc6)c45)CC3)(C1)C2]

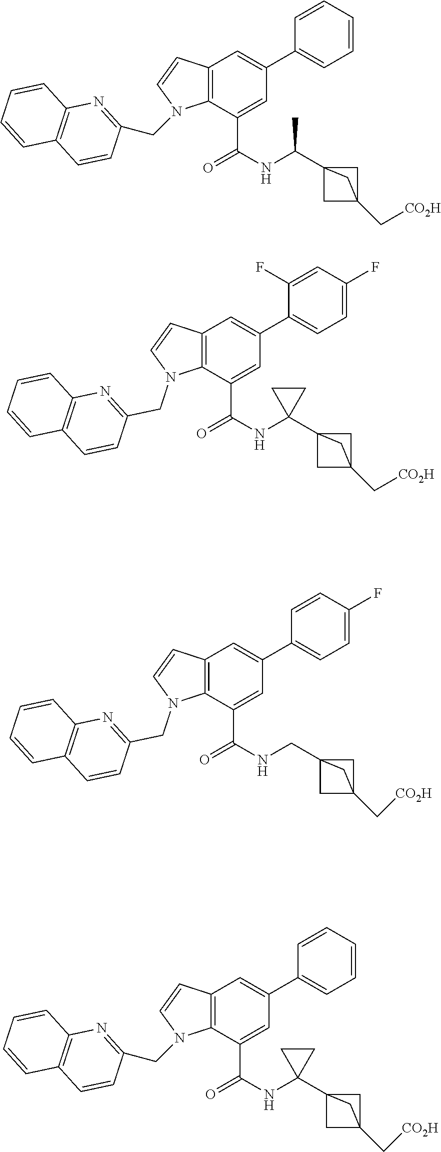

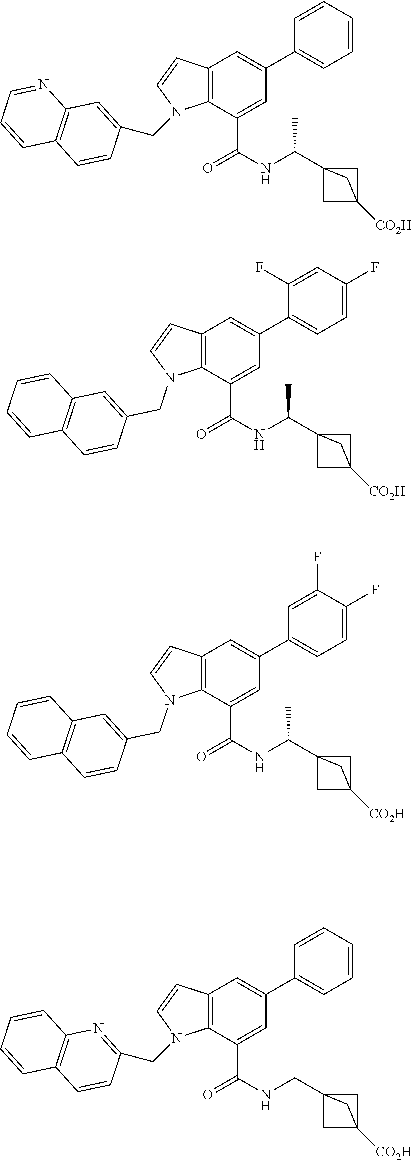

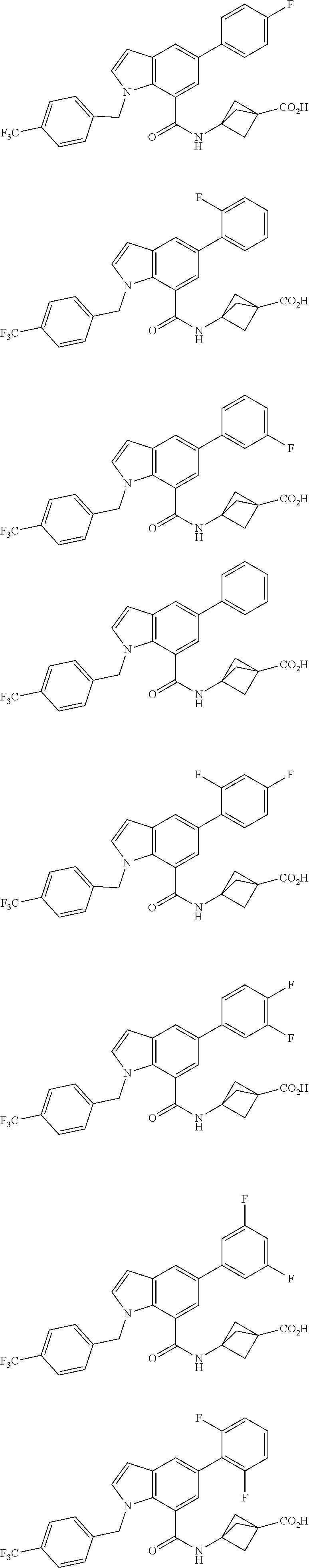

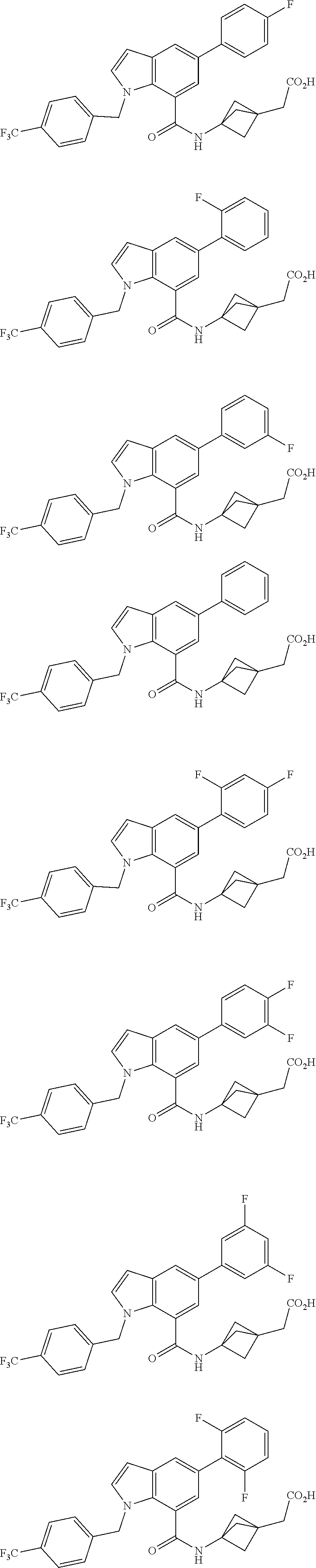

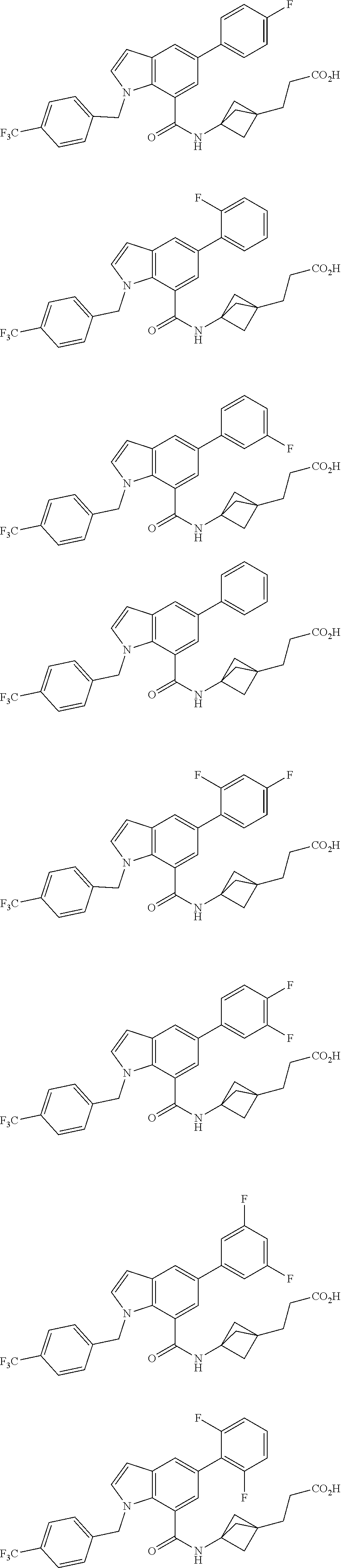

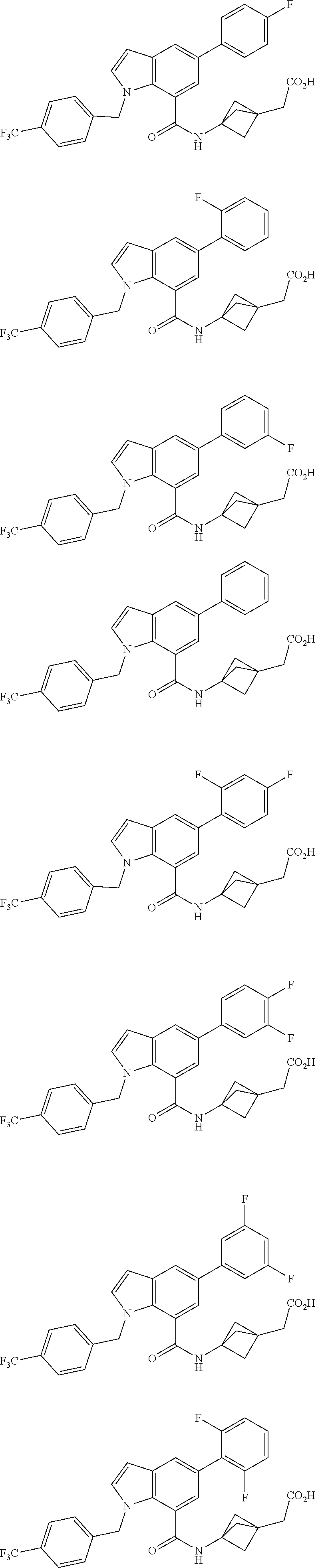

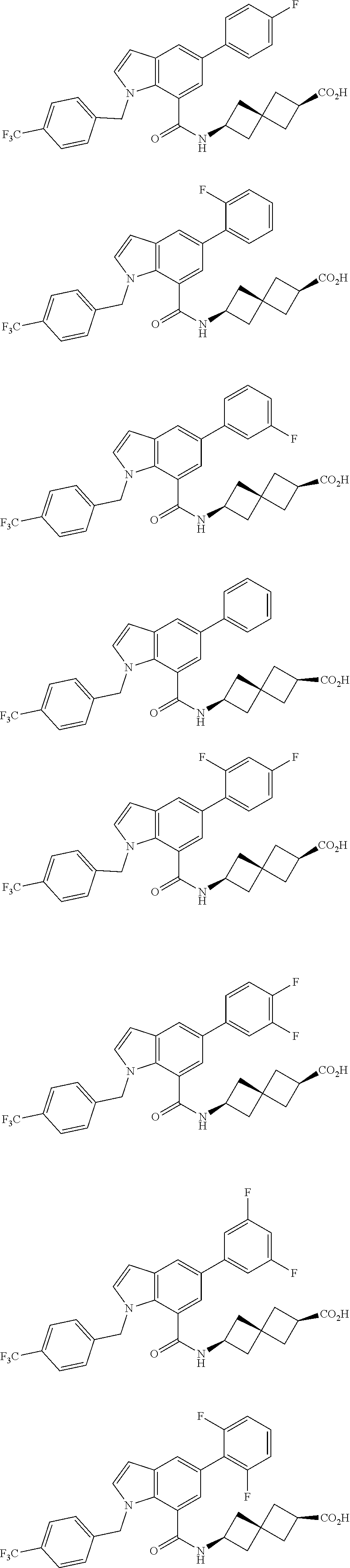

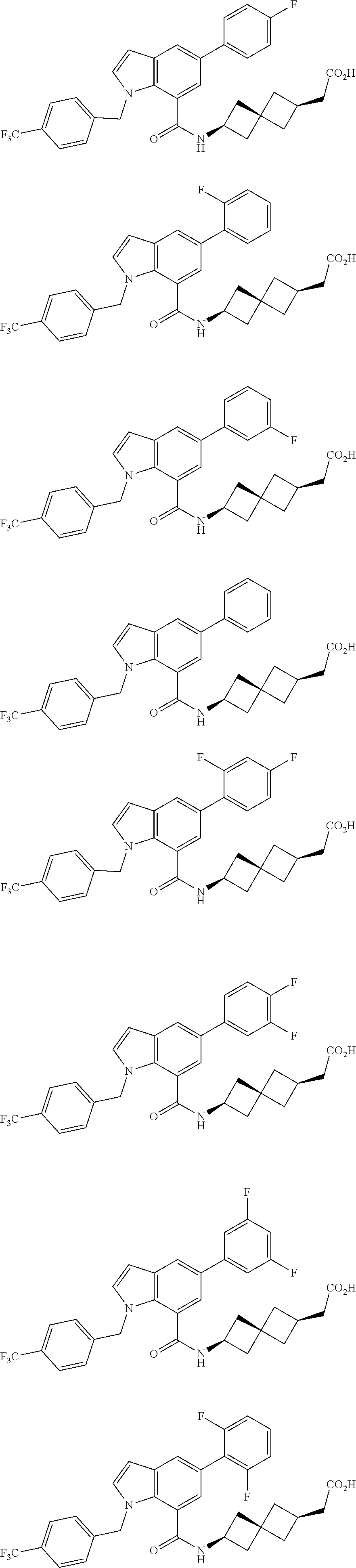

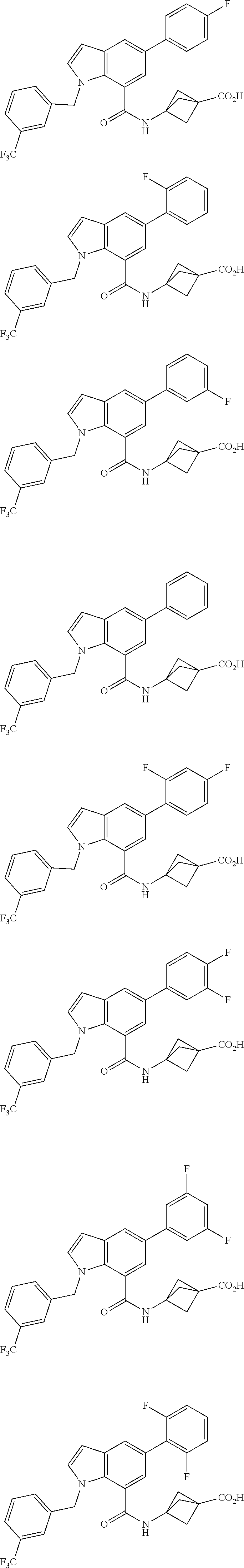

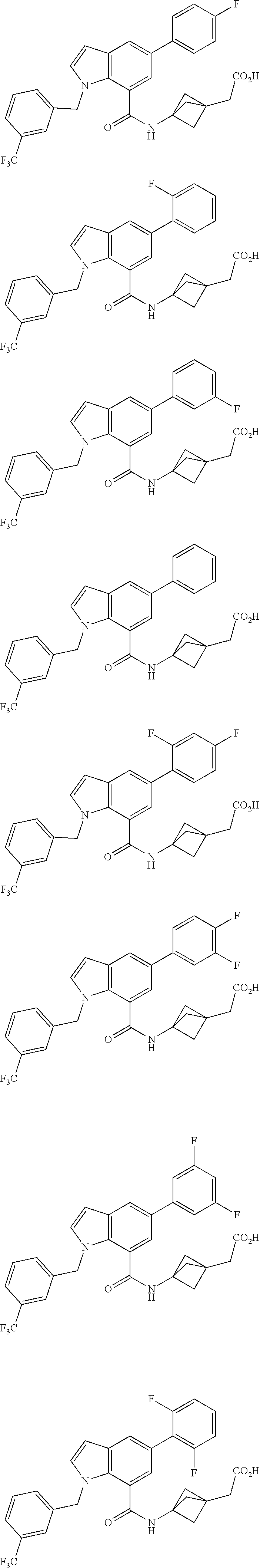

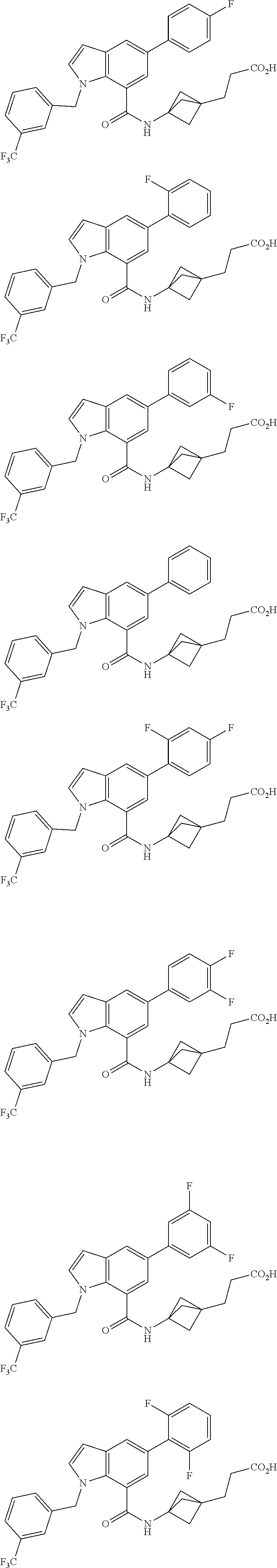

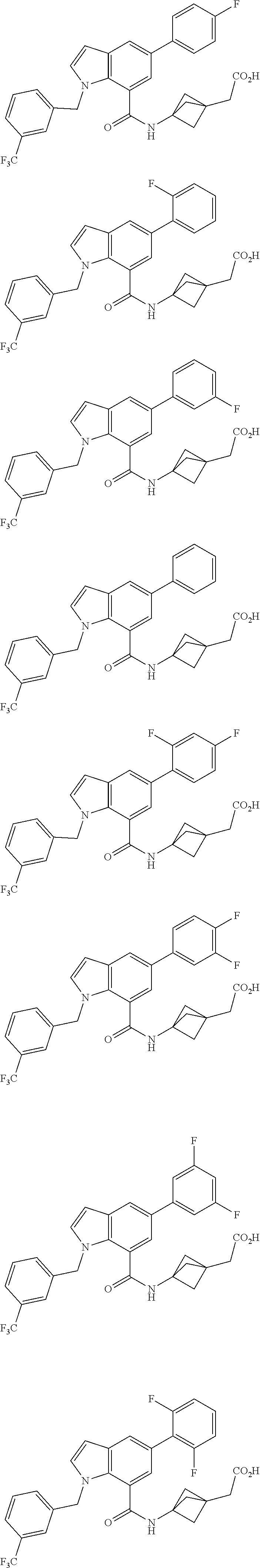

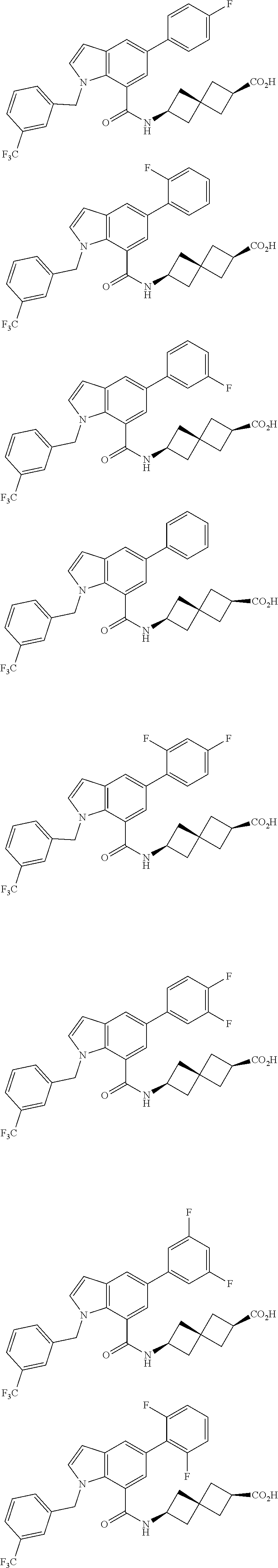

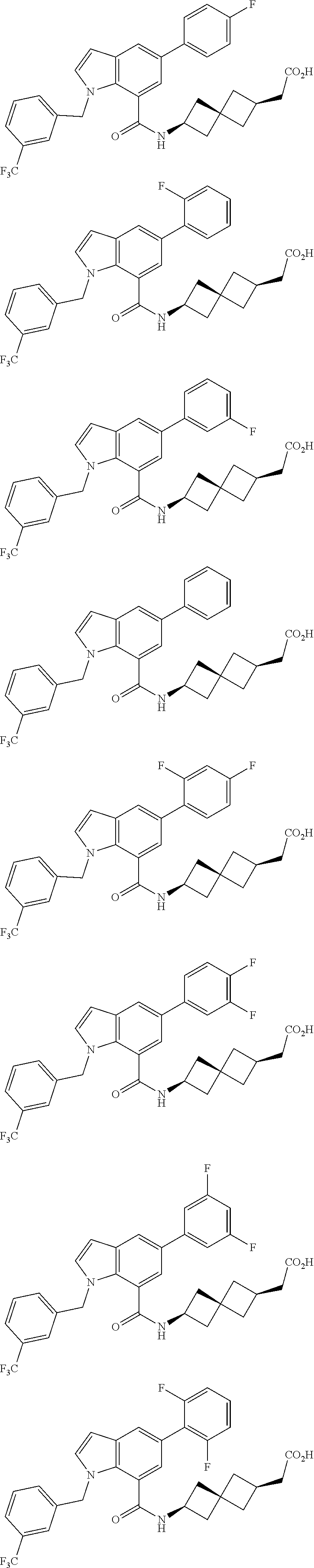

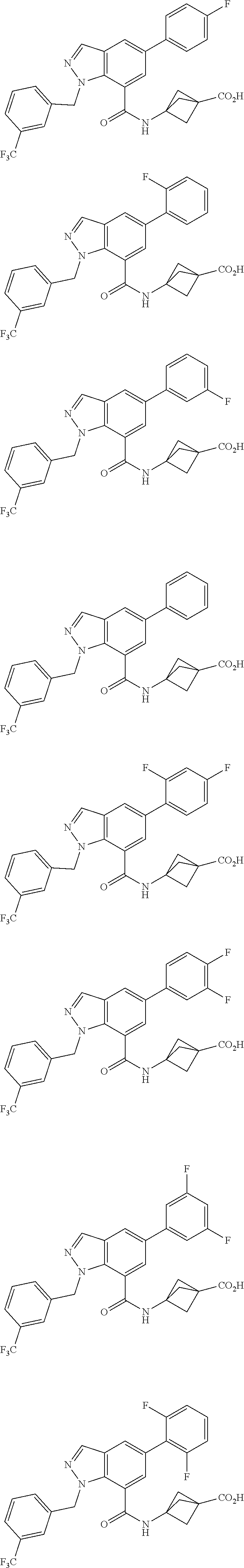

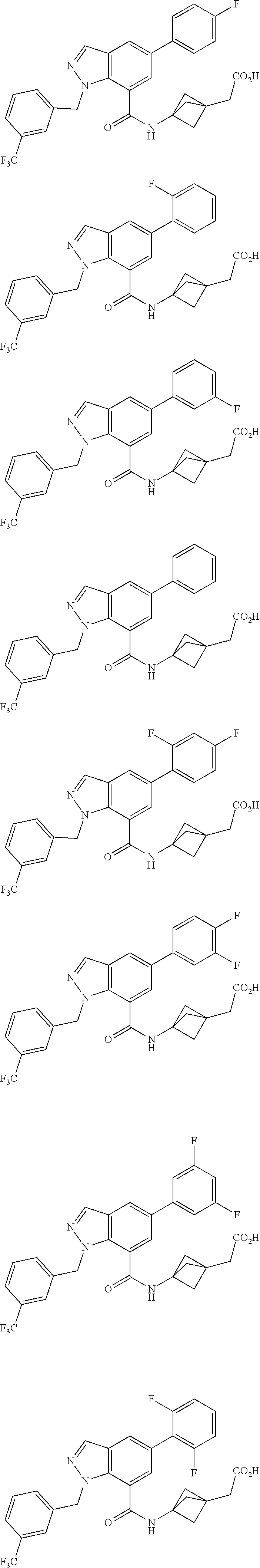

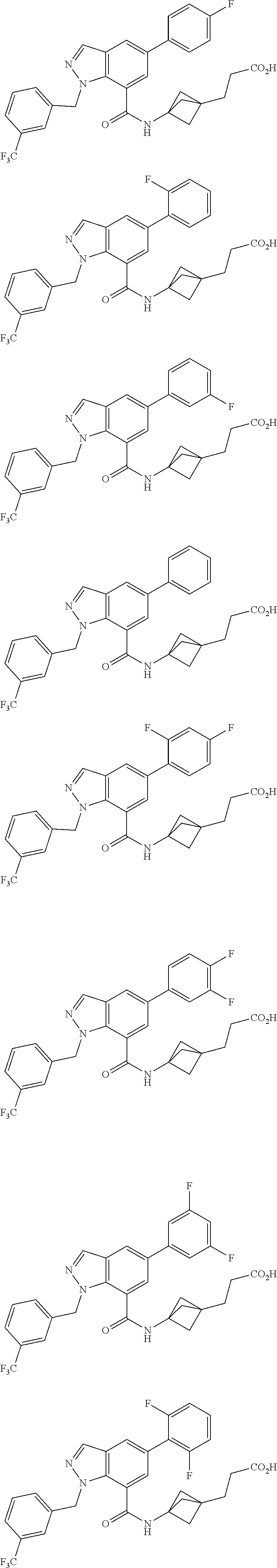

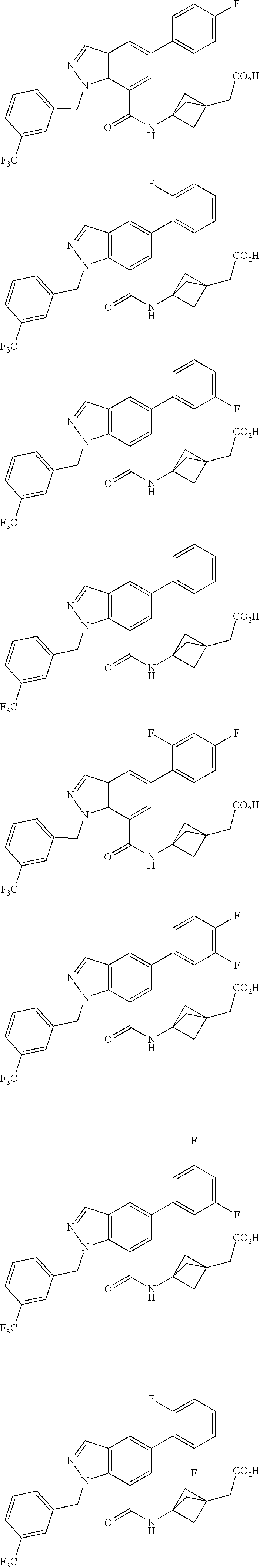

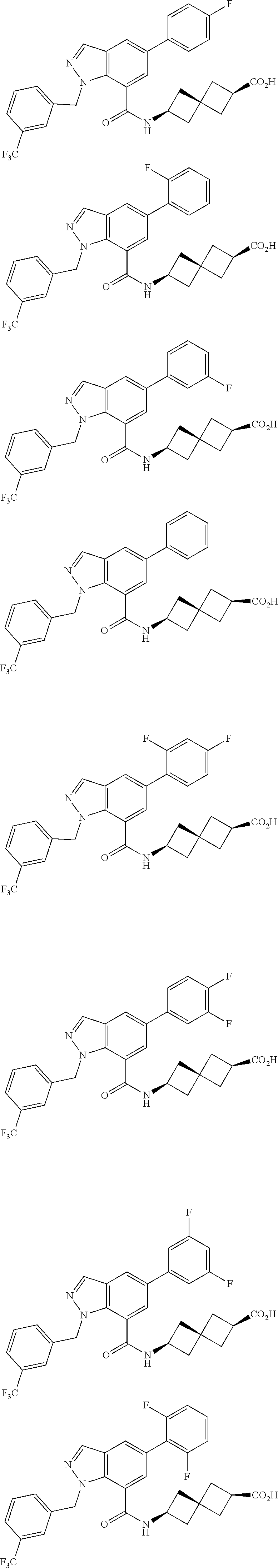

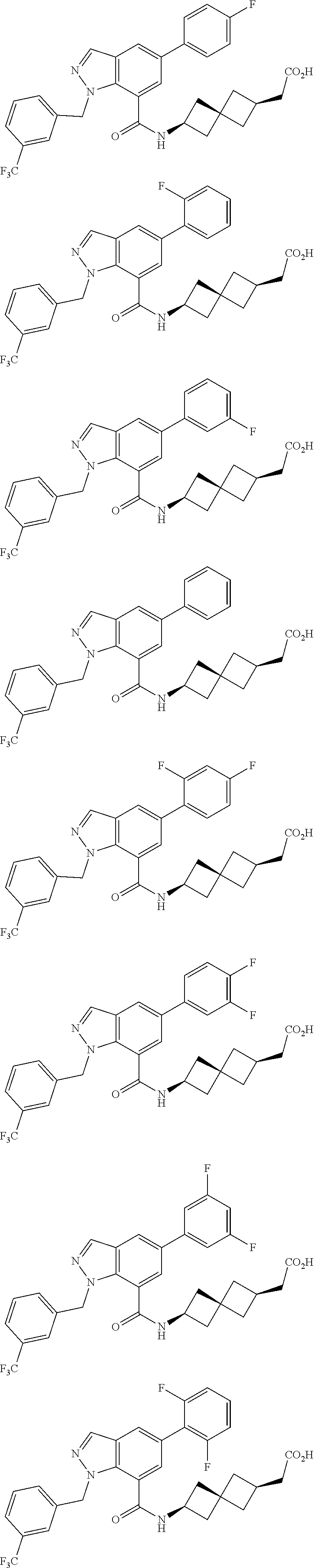

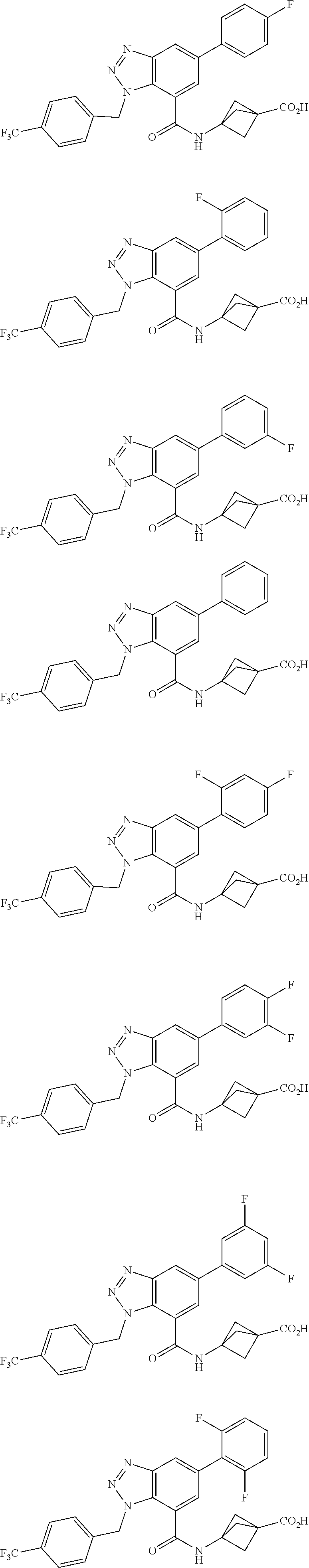

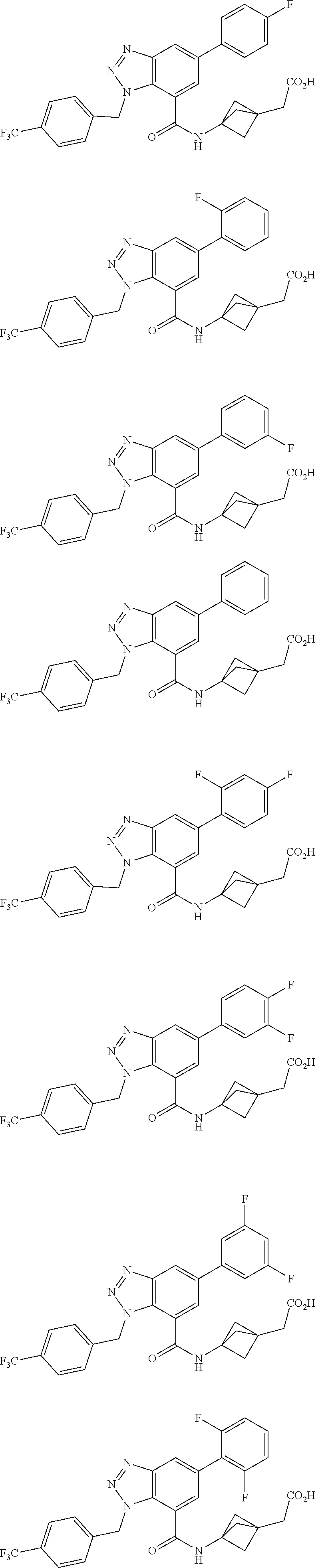

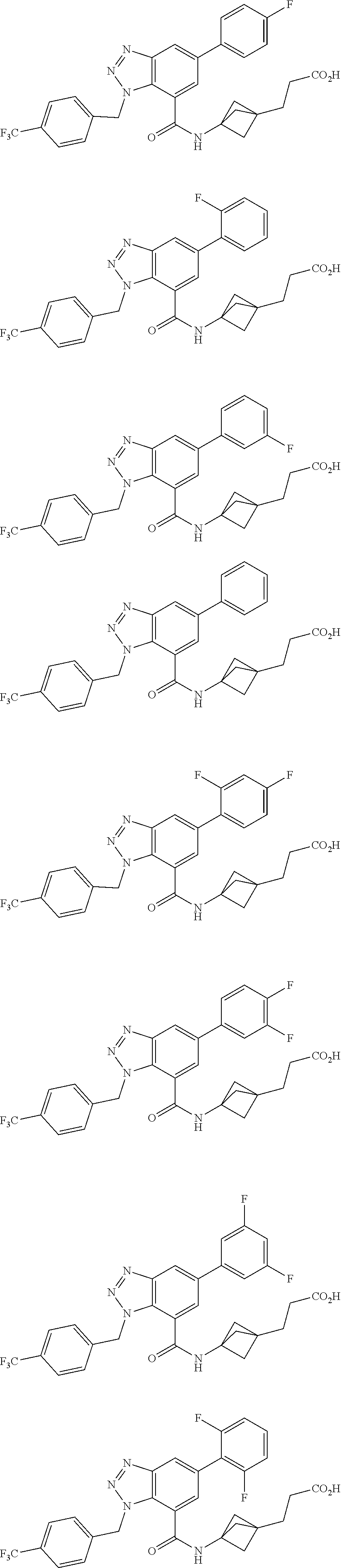

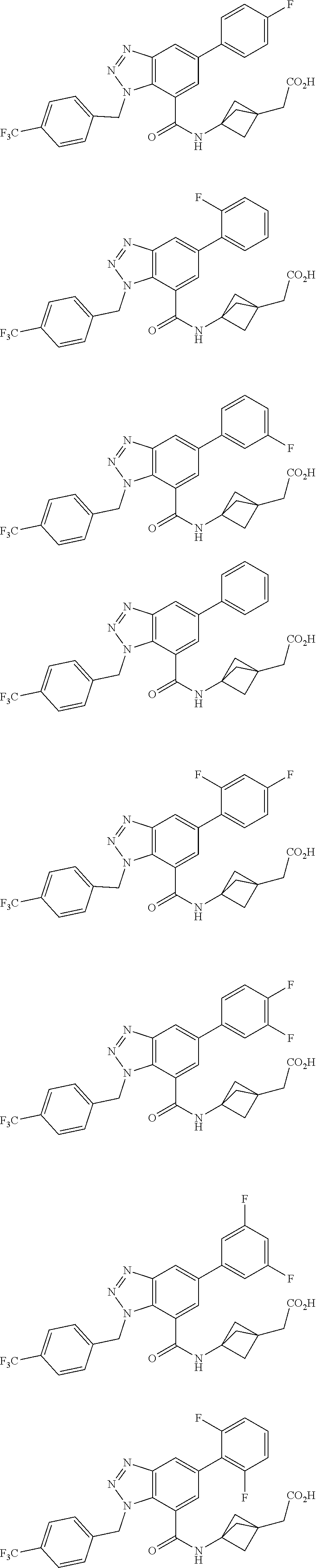

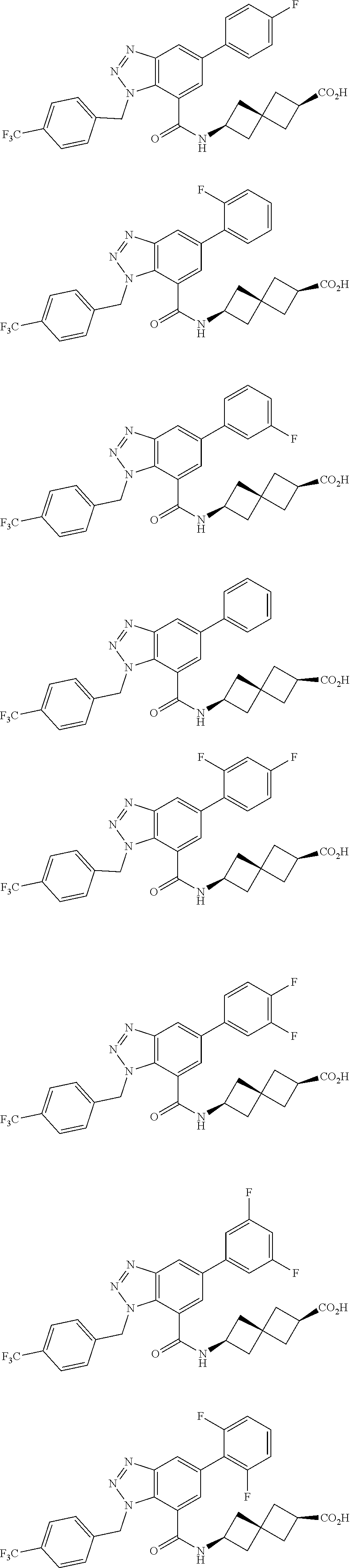

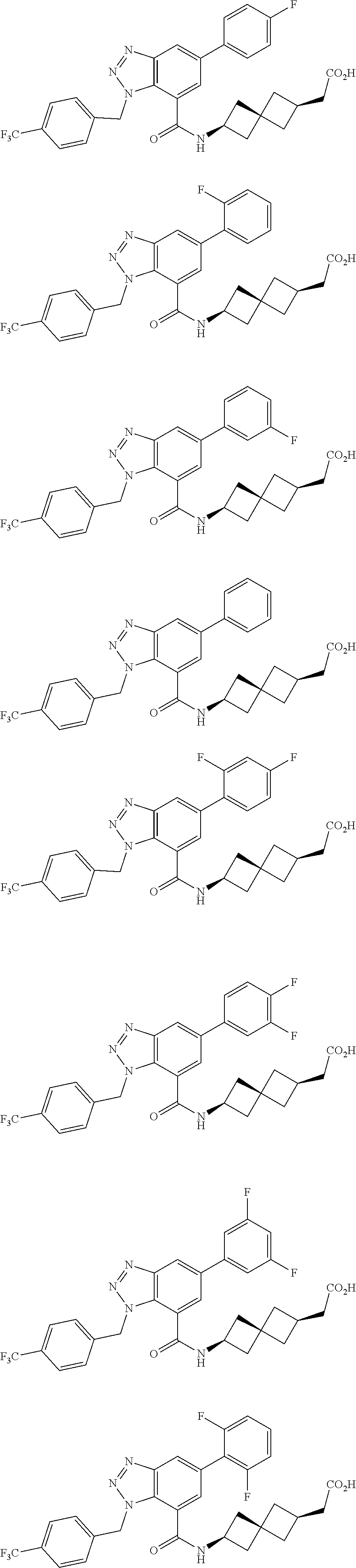

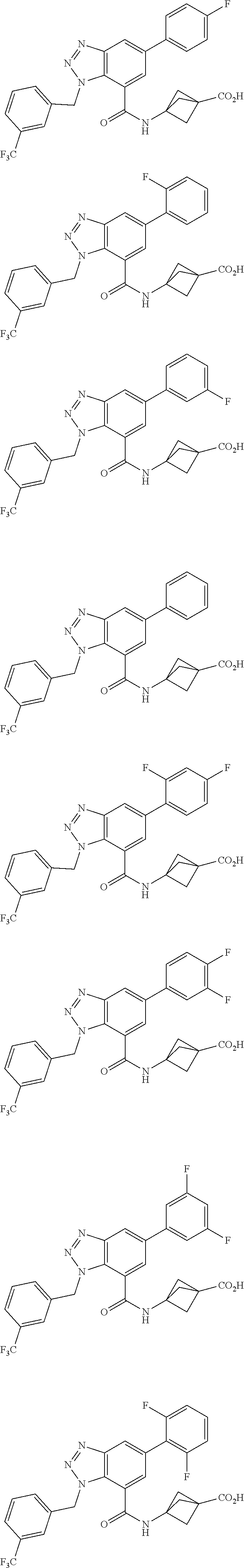

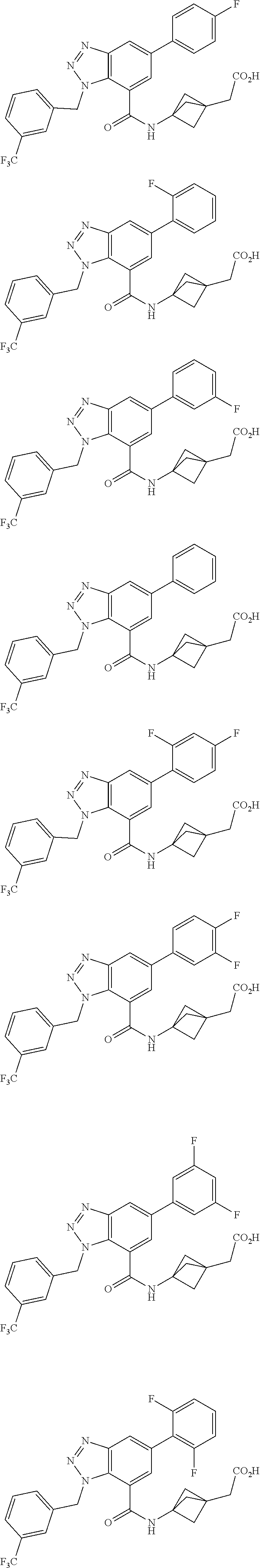

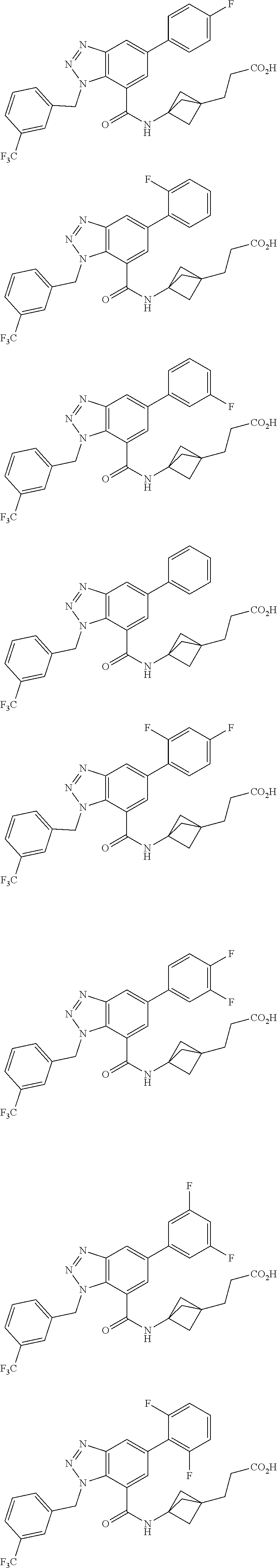

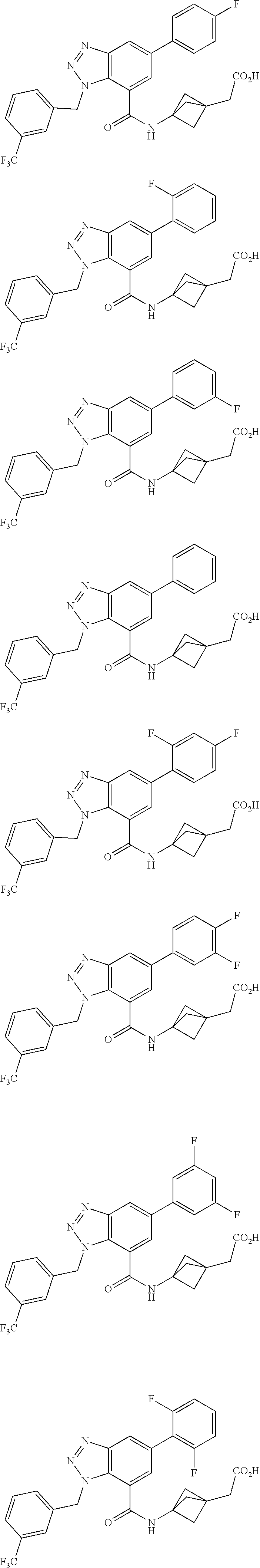

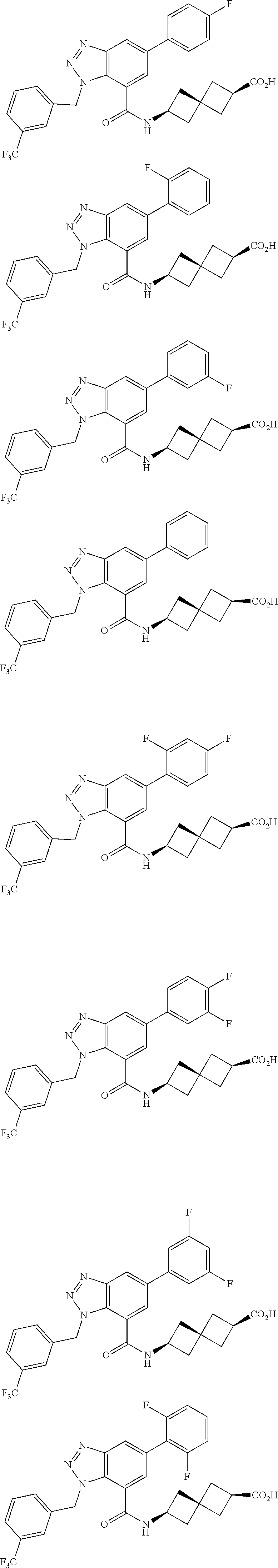

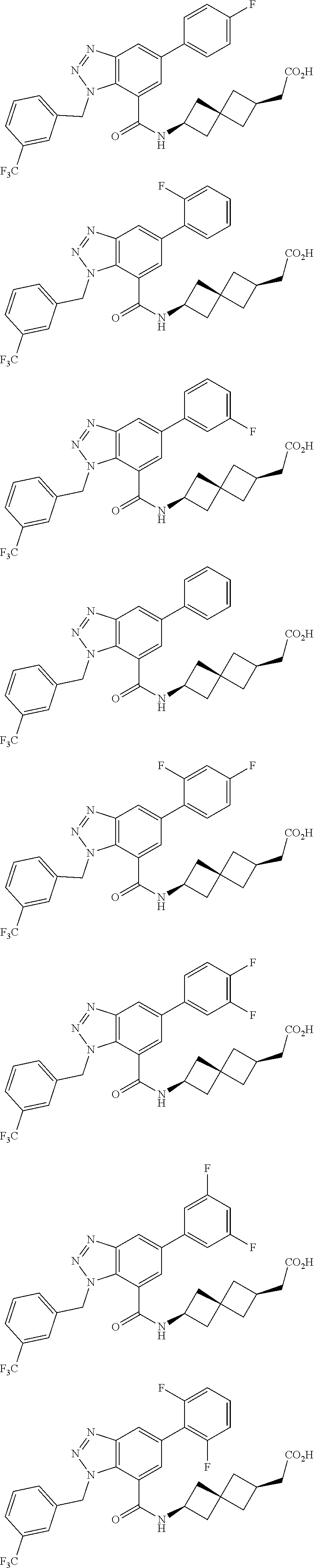

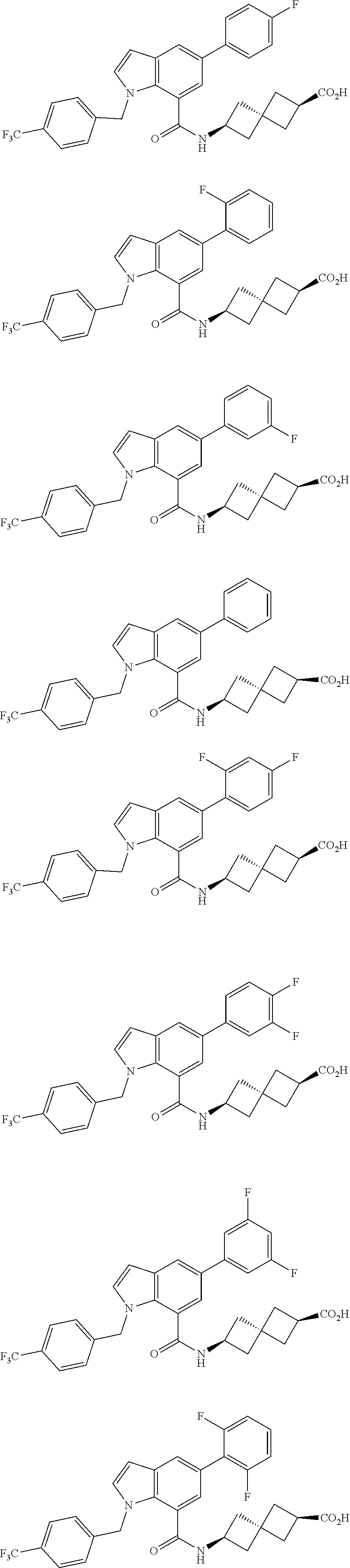

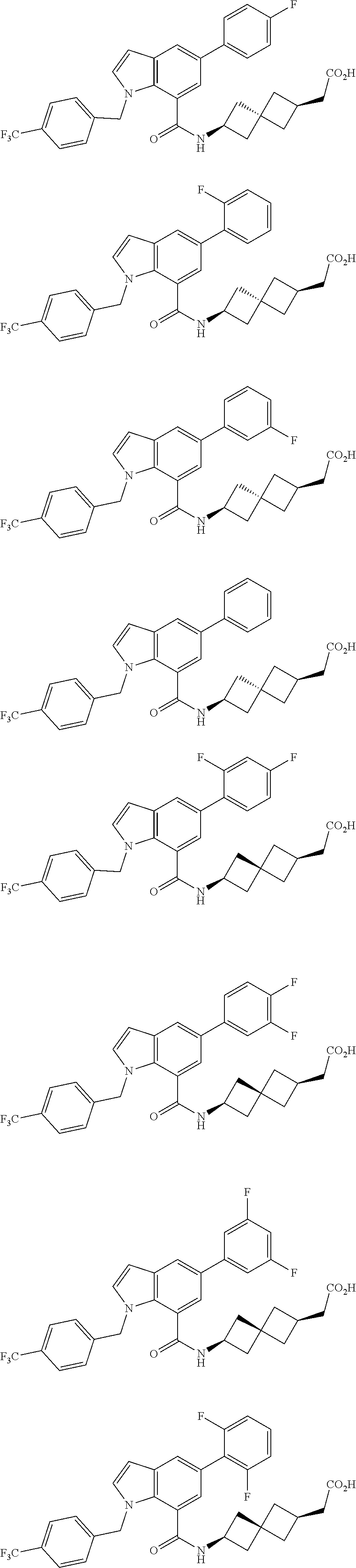

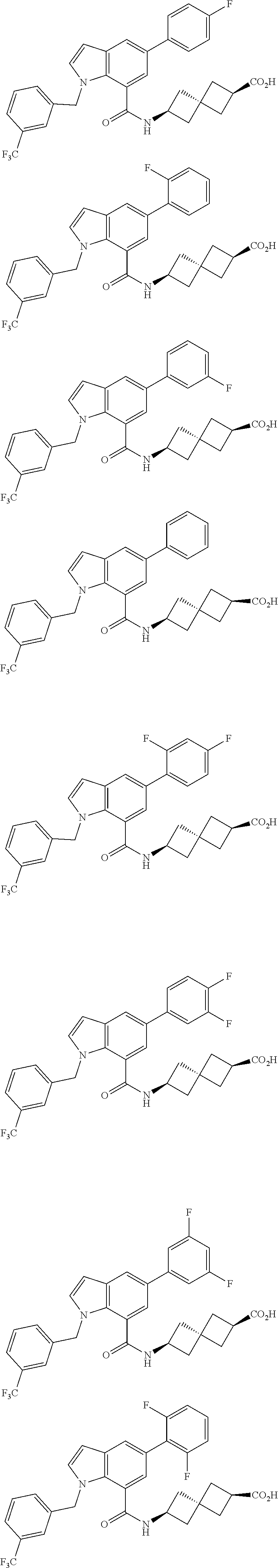

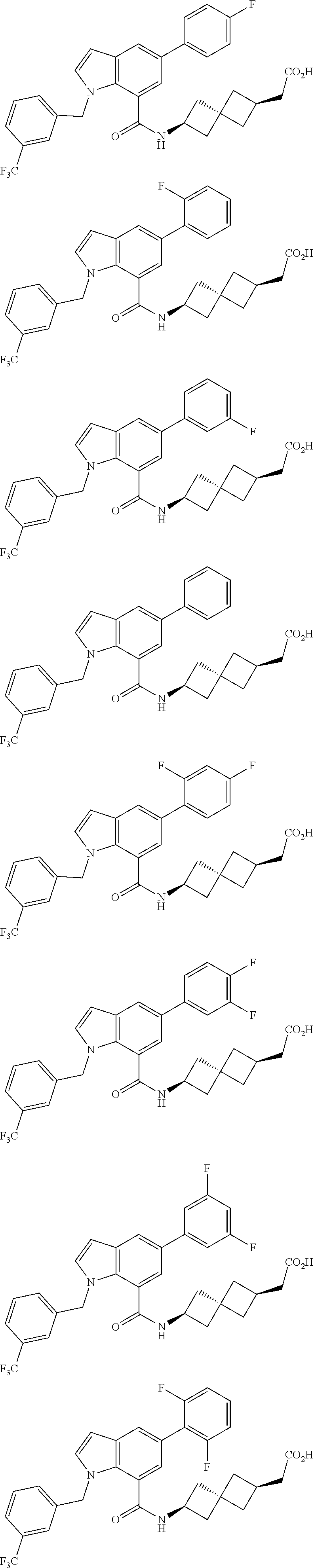

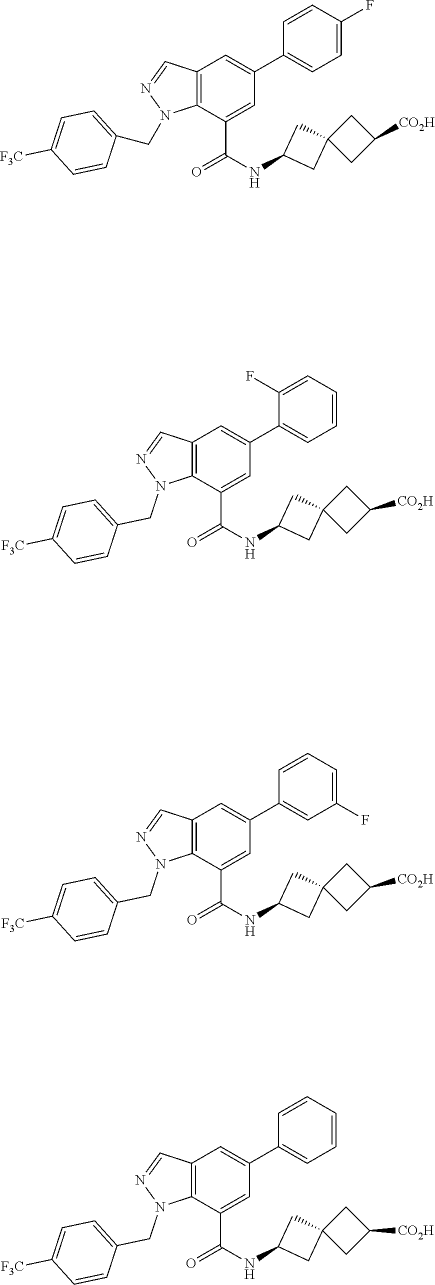

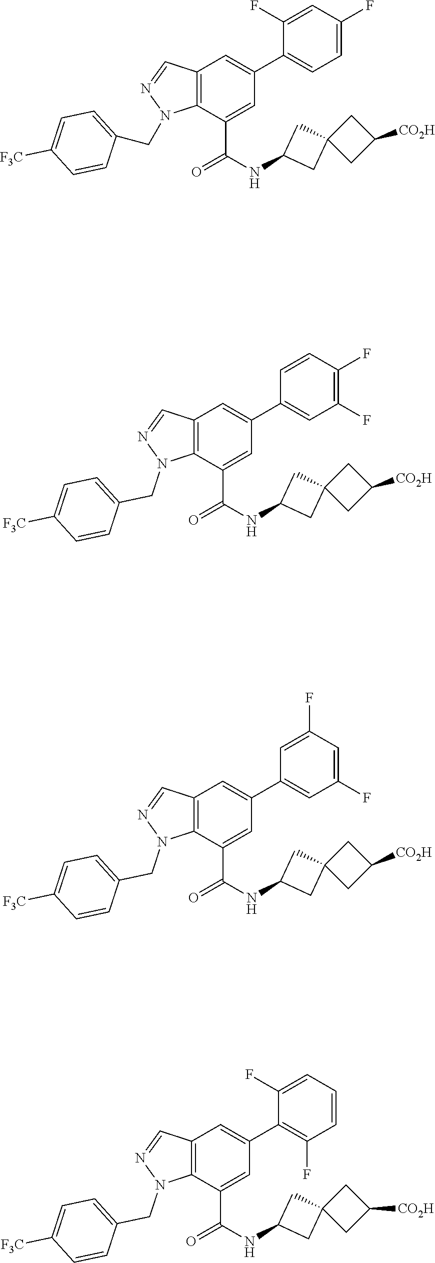

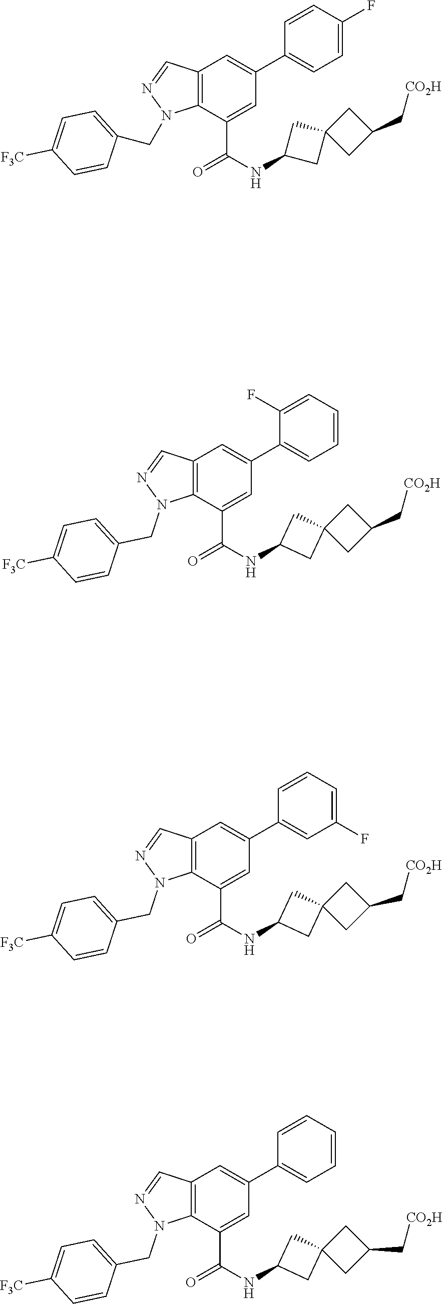

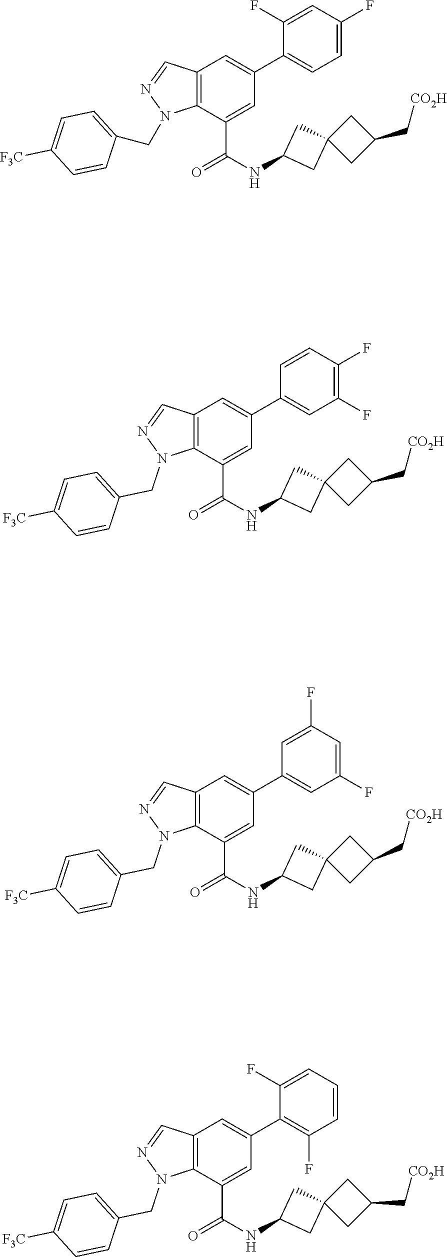

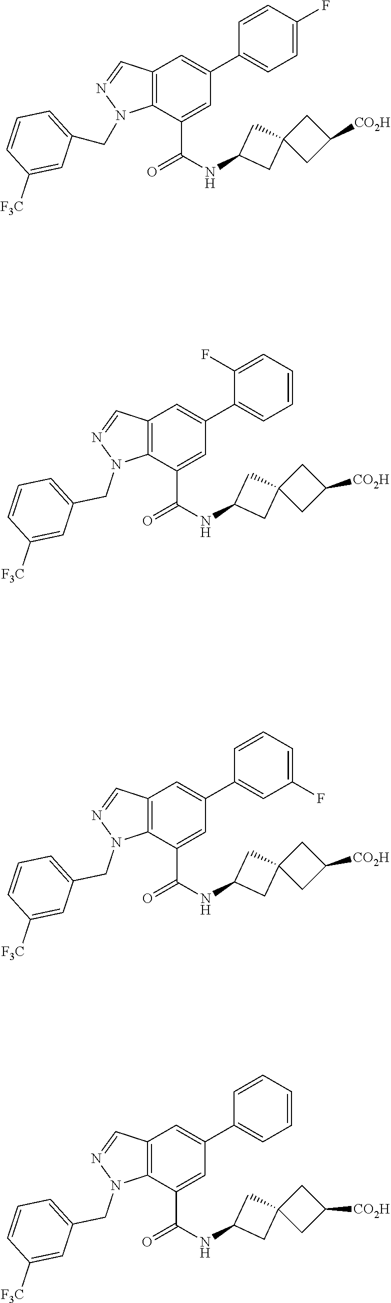

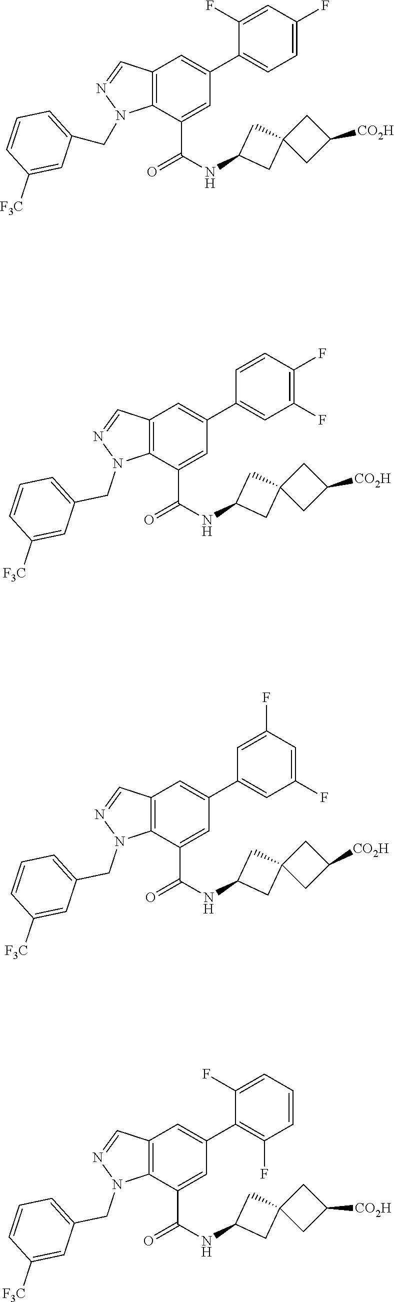

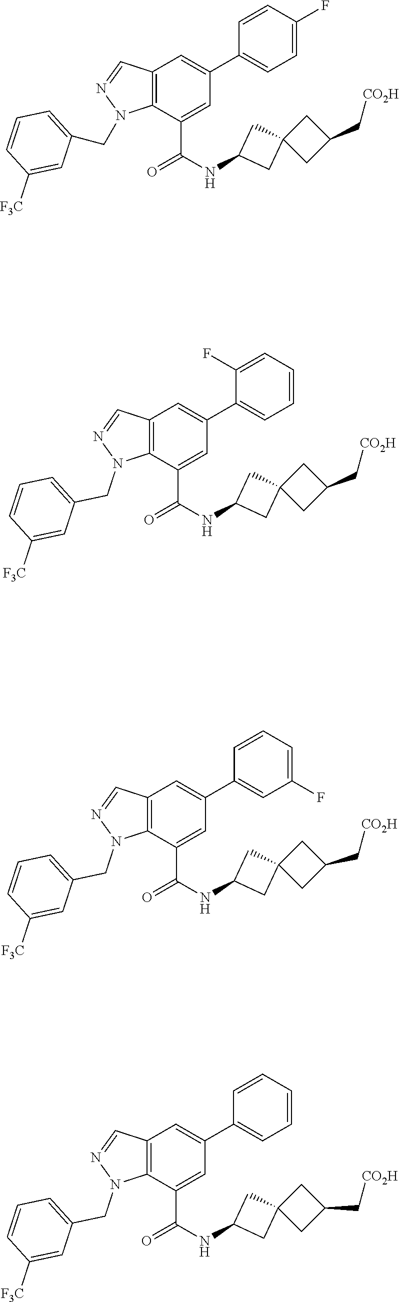

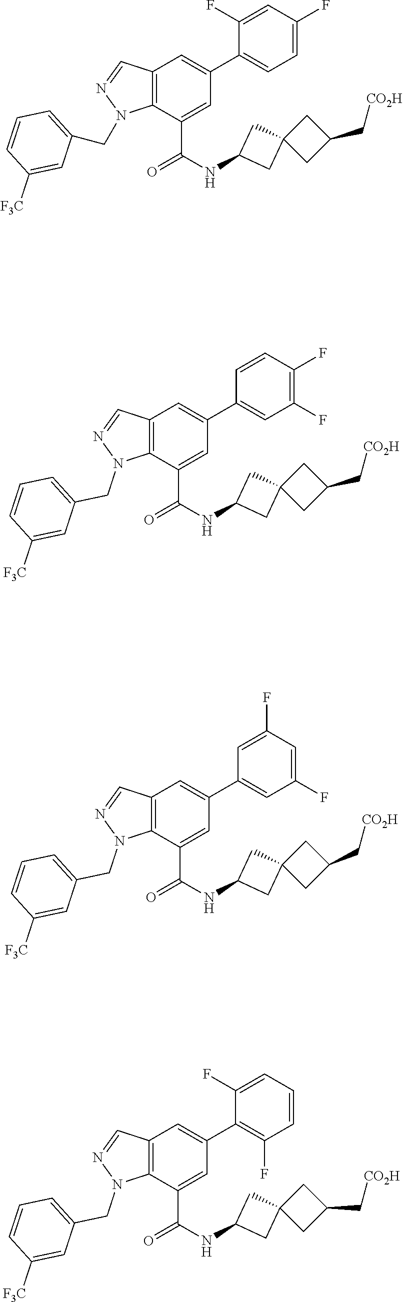

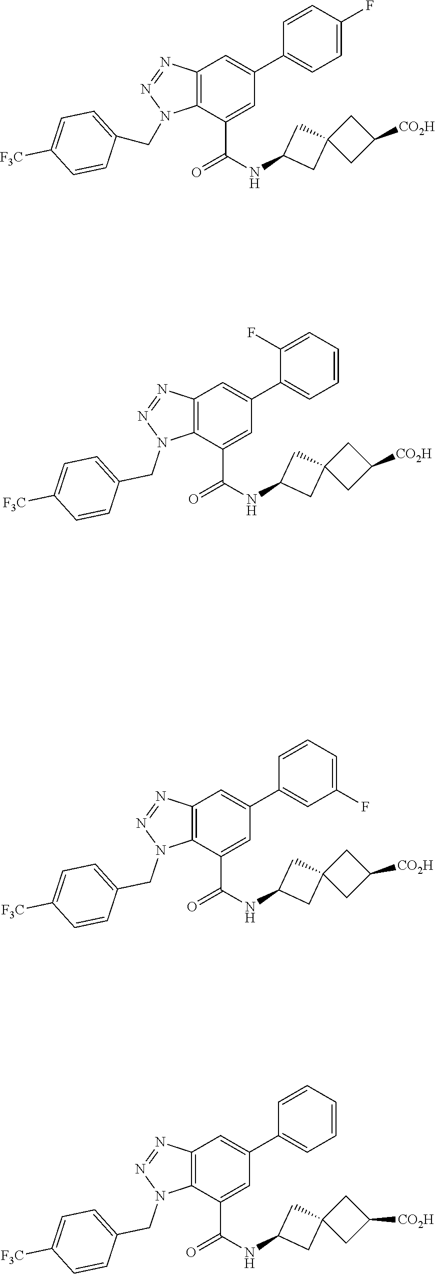

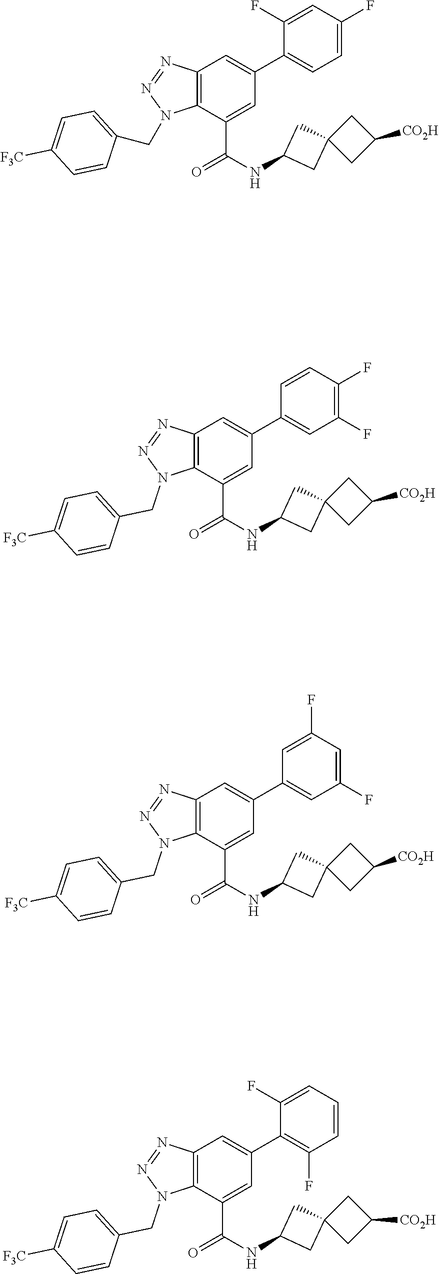

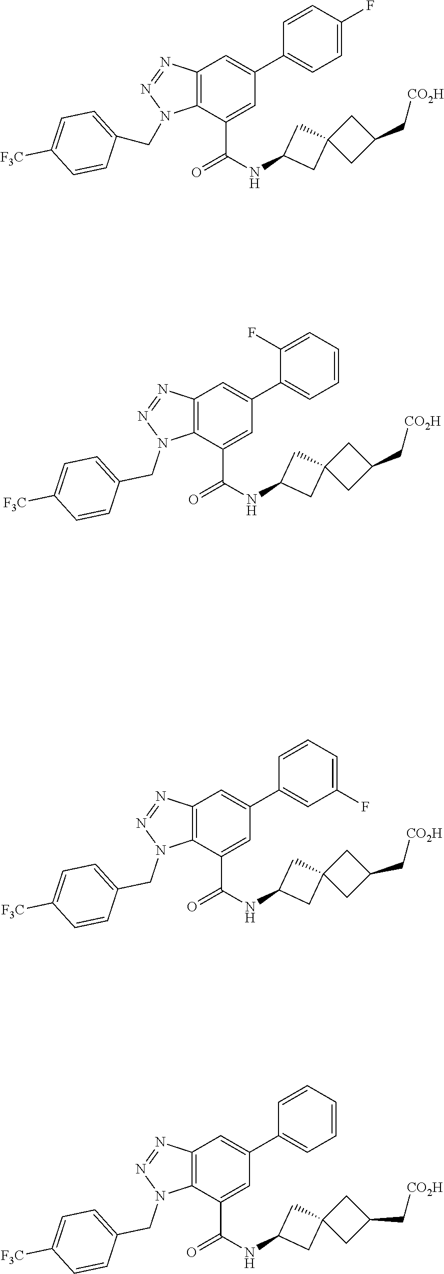

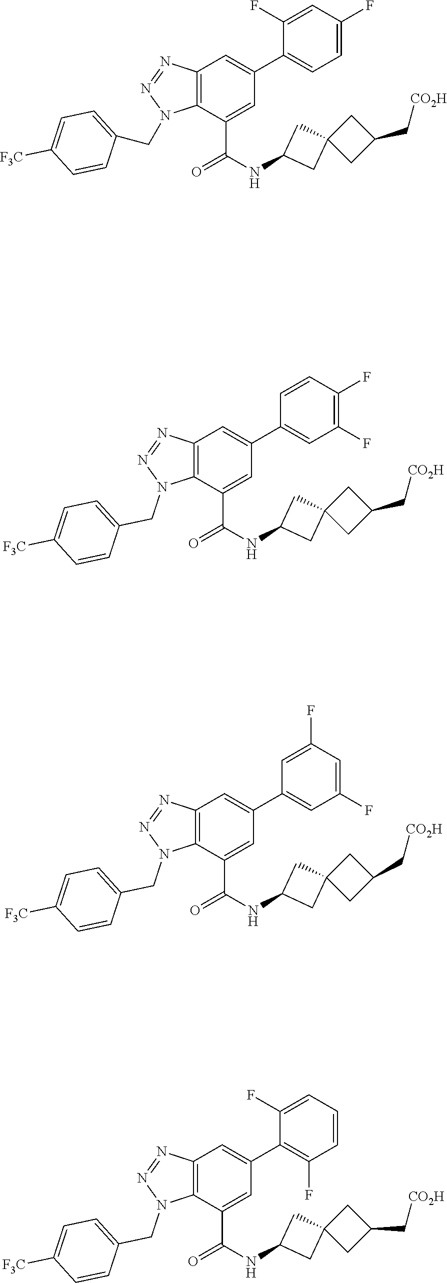

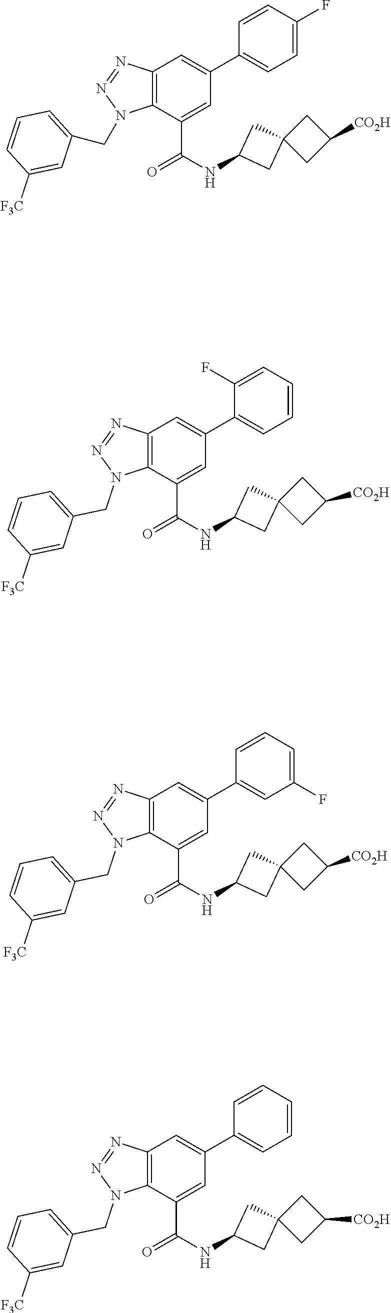

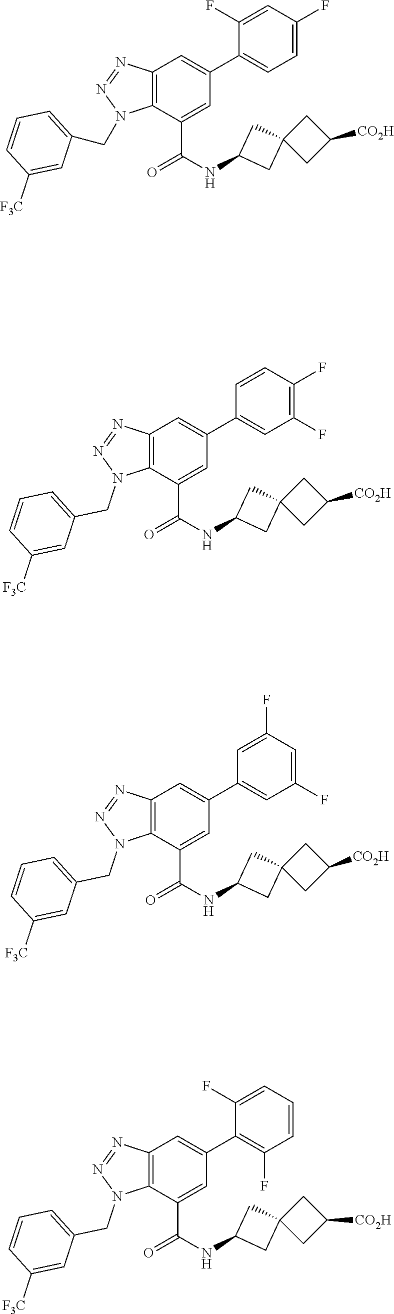

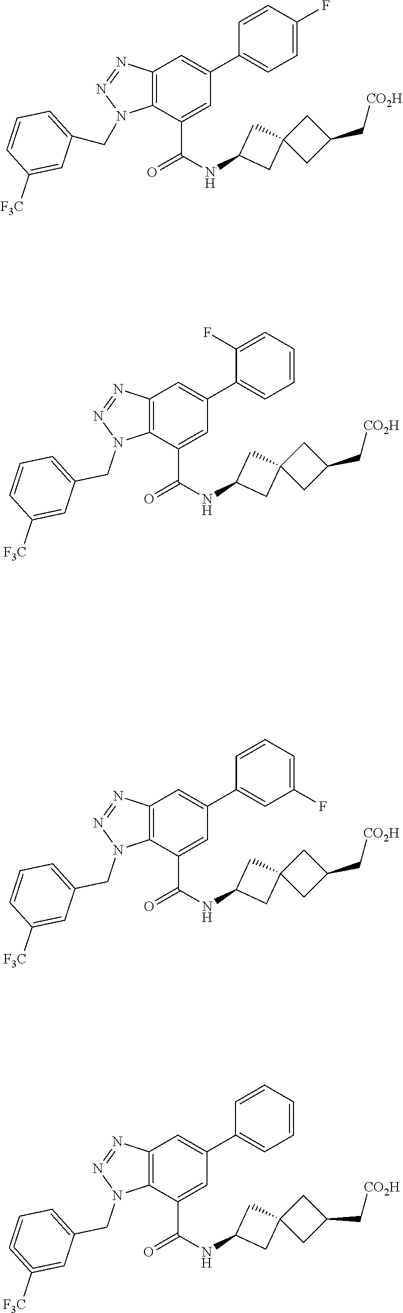

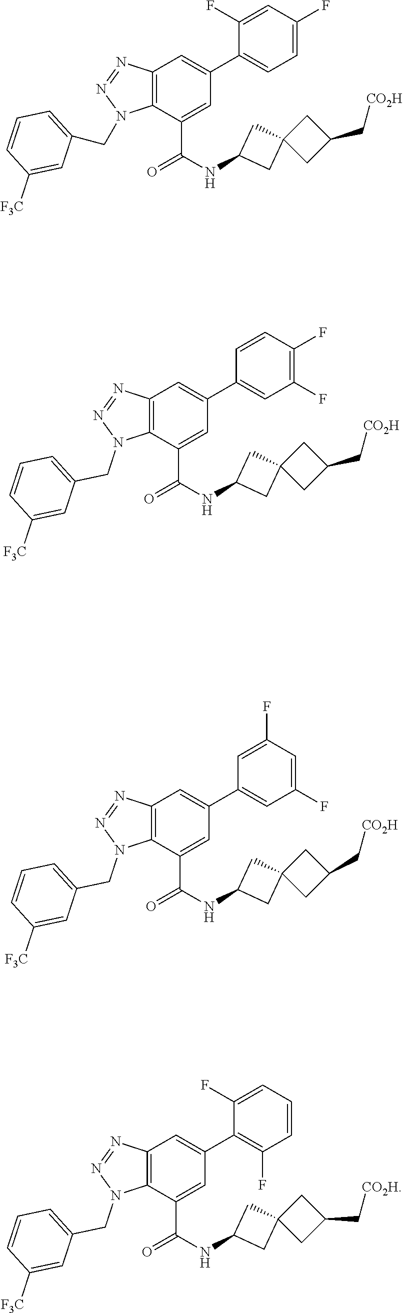

29. A method of treating a human or animal subject suffering from a condition mediated by the action of PGE2 receptors, via administering to said subject an effective amount of a compound according to claim 16, wherein said disease or pathological condition is selected from the group consisting of cancer, pain, neurodegenerative diseases, osteoarthritis, rheumatoid arthritis, endometriosis and kidney diseases.

30. The method according to claim 29, wherein the compound combining with an antibody for treating cancer, wherein the antibody is selected from the group consisting of antibodies against CTLA4, PD-L1, PD-1, or in combination with a small molecule drug such as kinase inhibitors, A2A receptor antagonists, IDO inhibitors, TDO inhibitors, HDAC inhibitors, PI3K/AKT pathway inhibitors, endocrine/hormone therapeutics, other chemotherapeutic agents, or antimetabolites.

31. The compound according to claim 16, wherein said compound of Formula I is 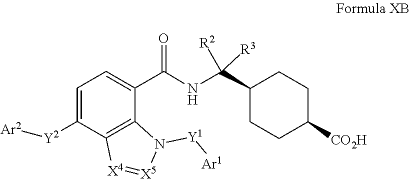
wherein X4 and X5 is are independently N or CR6; R2, R3, R6, Y1, Y2, Ar1 and Ar2 are defined as in Formula I, When R2 and R3 are different groups, Formula XB includes both R and S enantiomers.
